(19) United States
(12) Patent Application Publication
(10) Pub. No.: US 2025/0051338 A1
(43) Pub. Date: Feb. 13, 2025

(54) TYK2 DEGRADERS AND USES THEREOF

(71) Applicant: Kymera Therapeutics, Inc., Watertown, MA (US)

(72) Inventors: Isaac MARX, Watertown, MA (US); Christopher M. YATES, Watertown, MA (US); Xiao ZHU, Watertown, MA (US); Lewis Dale PENNINGTON, Watertown, MA (US); Eamon COMER, Watertown, MA (US); Melissa FORD, Watertown, MA (US)

(21) Appl. No.: 18/704,633

(22) PCT Filed: Oct. 24, 2022
(86) PCT No.: PCT/US2022/047570

§ 371 Date: Apr. 25, 2024

Related U.S. Application Data
(60) Provisional application No. 63/271,648, filed on Oct. 25, 2021.

(51) Int. Cl. C07D 487/04; A61K 31/5025; A61K 31/506; C07D 401/14

(52) U.S. Cl. CPC C07D 487/04; A61K 31/5025; A61K 31/506; C07D 401/14

(57) ABSTRACT

The present invention provides compounds, compositions thereof, and methods of using the same. Compounds and compositions thereof that are useful, for example, for targeting, inhibiting, and/or degrading TYK2. In certain embodiments, provided are TYK2 inhibitors and/or degraders and methods of making same. More specifically, TYK2 degraders, compositions which comprise TYK2 degraders, and methods of treating TYK2-associated conditions are provided.

### CROSS REFERENCE TO RELATED APPLICATION

This application claims priority to U.S. Provisional Application No. 63/271,648, filed Oct. 25, 2021, which is hereby incorporated by reference in its entirety.

### TECHNICAL FIELD OF THE INVENTION

The present invention relates to compounds and methods useful for the modulation of tyrosine kinase 2 (TYK2) protein via ubiquitination and/or degradation by compounds according to the present invention. The invention also provides pharmaceutically acceptable compositions comprising compounds of the present invention and methods of using said compositions in the treatment of various disorders.

### BACKGROUND OF THE INVENTION

Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

There are over 600 E3 ubiquitin ligases which facilitate the ubiquitination of different proteins in vivo, which can be divided into four families: HECT-domain E3s, U-box E3s, monomeric RING E3s and multi-subunit E3s. See generally Li et al. (PLOS One, 2008, 3, 1487) titled “Genome-wide and functional annotation of human E3 ubiquitin ligases identifies MULAN, a mitochondrial E3 that regulates the organelle's dynamics and signaling.”; Berndsen et al. (Nat. Struct. Mol. Biol., 2014, 21, 301-307) titled “New insights into ubiquitin E3 ligase mechanism”; Deshaies et al. (Ann. Rev. Biochem., 2009, 78, 399-434) titled “RING domain E3 ubiquitin ligases.”; Spratt et al. (Biochem. 2014, 458, 421-437) titled “RBR E3 ubiquitin ligases: new structures, new insights, new questions.”; and Wang et al. (Nat. Rev. Cancer, 2014, 14, 233-347) titled “Roles of F-box proteins in cancer.”

UPP plays a key role in the degradation of short-lived and regulatory proteins important in a variety of basic cellular processes, including regulation of the cell cycle, modulation of cell surface receptors and ion channels, and antigen presentation. The pathway has been implicated in several forms of malignancy, in the pathogenesis of several genetic diseases (including cystic fibrosis, Angelman's syndrome, and Liddle syndrome), in immune surveillance/viral pathogenesis, and in the pathology of muscle wasting. Many diseases are associated with an abnormal UPP and negatively affect cell cycle and division, the cellular response to stress and to extracellular modulators, morphogenesis of neuronal networks, modulation of cell surface receptors, ion channels, the secretory pathway, DNA repair and biogenesis of organelles.

Aberrations in the process have recently been implicated in the pathogenesis of several diseases, both inherited and acquired. These diseases fall into two major groups: (a) those that result from loss of function with the resultant stabilization of certain proteins, and (b) those that result from gain of function, i.e. abnormal or accelerated degradation of the protein target.

The UPP is used to induce selective protein degradation, including use of fusion proteins to artificially ubiquitinate target proteins and synthetic small-molecule probes to induce proteasome dependent degradation. Bifunctional compounds composed of a target protein-binding ligand and an E3 ubiquitin ligase ligand, induced proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins (Crews C, Chemistry & Biology, 2010, 17(6):551-555; Schnnekloth J S Jr., Chembiochem, 2005, 6(1):40-46).

TYK2 is an enzyme encoded by the TYK2 gene in humans and a member of the Janus Kinase (JAKs) family of proteins. TYK2 is involved IL-12, IL-23 and type I-interferon (IFN) signaling (Morris R, et al., Protein Science, Volume: 27, Issue: 12, Pages: 1984-2009, 2018). Human genetic studies suggest that TYK2 inhibition can be broadly beneficial for treating autoimmune and inflammatory diseases (Dendrou C, et al., Science Translational Medicine, Vol 8, Issue 363, p. 363ra149 2016).

An ongoing need exists in the art for effective treatments for diseases, especially autoimmune and inflammatory diseases and disorders mediated by pro-inflammatory molecules such as IFN-α/β, IL-12, and IL-23 without JAK 1/2 inhibition which may cause on-target adverse events. As such, small molecule therapeutic agents that leverage E3 ligase mediated protein degradation to pro-inflammatory associated proteins such as tyrosine kinase 2 (TYK2) while potentially sparing molecules involved in wound healing and protection against microbes such as IL-10 and IL-22 hold promise as therapeutic agents for the treatment of conditions such as Crohn's disease and ulcerative colitis. Accordingly, there remains a need to find compounds that are TYK2 degraders useful as therapeutic agents.

### SUMMARY OF THE INVENTION

The present application relates novel bifunctional compounds, which function to recruit TYK2 protein to E3 ubiquitin ligase for degradation, and methods of preparation and uses thereof. In particular, the present disclosure provides bifunctional compounds, which find utility as modulators of targeted ubiquitination of TYK2 protein, which are then degraded and/or otherwise inhibited by the bifunctional compounds as described herein. Also provided are monovalent compounds, which find utility as inducers of targeted ubiquitination of TYK2 protein, which are then degraded and/or otherwise inhibited by the monovalent compounds as described herein. An advantage of the compounds provided herein is that a broad range of pharmacological activities is possible, consistent with the degradation/inhibition of TYK2 protein. In addition, the description provides methods of using an effective amount of the compounds as described herein for the treatment or amelioration of a disease condition, such as cancer.

The present application further relates to targeted degradation of TYK2 protein through the use of bifunctional molecules, including bifunctional molecules that link a cereblon-binding moiety to a ligand that binds TYK2 protein.

It has now been found that compounds of this invention, and pharmaceutically acceptable compositions thereof, are effective as degraders of TYK2 protein. Such compounds have the general Formula (I):

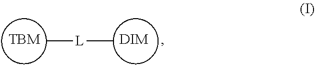

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with regulation of signaling pathways implicating TYK2 protein. Such diseases, disorders, or conditions include those described herein.

Compounds provided by this invention are also useful for the study of TYK2 protein in biological and pathological phenomena; the study of intracellular signal transduction pathways occurring in bodily tissues; and the comparative evaluation of new TYK2 inhibitors or TYK2 degraders or other regulators of cell cycling, metastasis, angiogenesis, and immune cell evasion, in vitro or in vivo.

### DETAILED DESCRIPTION OF CERTAIN EMBODIMENTS

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

Unless defined otherwise, all technical and scientific terms used herein have the same meaning as commonly understood by one of ordinary skill in the art to which this invention belongs.

### General Description of Certain Embodiments

Compounds of the present invention, and compositions thereof, are useful as degraders and/or inhibitors of one or more TYK2 proteins.

In one aspect, the present invention provides a compound of Formula (I):

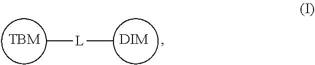

or a pharmaceutically acceptable salt thereof, wherein:
TBM is a TYK binding moiety capable of binding to TYK2 protein;
L is a bivalent moiety that connects TBM to DIM; and
DIM is a degradation-inducing moiety selected from a ligase binding moiety (LBM) and a lysine mimetic, or a hydrogen atom.

### Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. In some embodiments, a carbocyclic ring may be a 5-12 membered bicyclic, bridged bicyclic, or spirocyclic ring. A carbocyclic ring may include one or more oxo (═O) or thioxo (═S) substituent. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 6-12 (e.g., 6-12) ring members and 0-4 heteroatoms independently selected from nitrogen, oxygen, and sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Without limitation, a bridged bicyclic group may contain two or more bridges, e.g., adamantanyl. Exemplary bridged bicyclics include but not limited to:

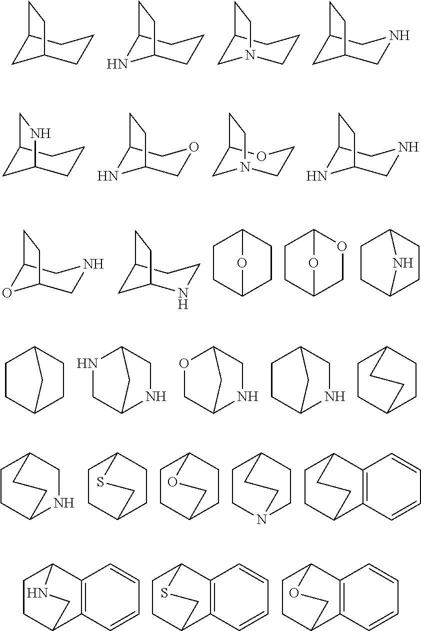

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

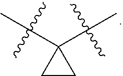 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 3- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. In some embodiments, a heterocyclic ring may be a 5-12 membered bicyclic, bridged bicyclic, or spirocyclic ring. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “substituted” moieties. In general, the term “substituted” means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)_{0- 4}OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, and sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, and sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR′₂, ═NNHC(O)R′, ═NNHC(O)OR′, ═NNHS(O)₂R′, ═NR′, ═NOR′, —O(C(R′₂))₂₋₃O—, or —S(C(R′₂))₂₋₃S—, wherein each independent occurrence of R′ is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR′₂)₂₋₃O—, wherein each independent occurrence of R′ is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur.

Suitable substituents on the aliphatic group of R′ include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. In some embodiments, the provided compounds are purified in salt form for convenience and/or ease of purification, e.g., using an acidic or basic mobile phase during chromatography. Salts forms of the provided compounds formed during chromatographic purification are comtemplated herein (e.g., diammonium salts) and are readily apparent to those having skill in the art.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention

As used herein, the term “provided compound” refers to any genus, subgenus, and/or species set forth herein.

The term “prodrug” refers to a compound that is made more active in vivo. The present compounds can also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use.

As used herein, the term “inhibitor” is defined as a compound that binds to and/or inhibits a TYK2 kinase with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

As used herein, the term “degrader” is defined as a heterobifunctional compound that binds to and/or inhibits both a TYK2 kinase and an E3 ligase with measurable affinity resulting in the ubiquitination and subsequent degradation of the TYK2 kinase. In certain embodiments, a degrader has an DC₅₀ of less than about 50 μM, less than about 1 μM, less than about 500 nM, less than about 100 nM, less than about 10 nM, or less than about 1 nM. As used herein, the term “monovalent” refers to a degrader compound without an appended E3 ligase binding moiety.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41:2596-99 and Sun et al., Bioconjugate Chem., 2006, 17:52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in a TYK2 kinase activity between a sample comprising a compound of the present invention, or composition thereof, and a TYK2 kinase, and an equivalent sample comprising a TYK2 kinase, in the absence of said compound, or composition thereof.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof, or (3) relieving the disease, i.e., causing regression of the state, disorder or condition or at least one of its clinical or sub-clinical symptoms. The benefit to a subject to be treated is either statistically significant or at least perceptible to the patient or to the physician. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a human.

As used herein the term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

Ranges can be expressed herein as from “about” or “approximately” one particular value and/or to “about” or “approximately” another particular value. When such a range is expressed, another embodiment includes from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily or degratorily active metabolite or residue thereof.

As used herein, the term “inhibitorily active metabolite or residue thereof” means that a metabolite or residue thereof is also an inhibitor of a TYK2 protein, or a mutant thereof.

As used herein, the term “degratorily active metabolite or residue thereof” means that a metabolite or residue thereof is also a degrader of a TYK2 protein, or a mutant thereof.

### TYK2 Binding Moiety (TBM)

In one aspect, the present invention provides a compound of Formula (I):

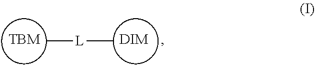

or a pharmaceutically acceptable salt thereof, wherein TBM is a TYK binding moiety capable of binding to TYK2 protein; L is a bivalent moiety that connects TBM to DIM; and DIM is a degradation-inducing moiety selected from a ligase binding moiety (LBM) and a lysine mimetic, or a hydrogen atom.

In some embodiments, TBM is a TYK2 protein binding moiety. Such TYK2 binders are well known to one of ordinary skill in the art and include those described in WO 2010/000089A1; WO 2012/000970A1; WO 2012/062704A1; WO/2012/066061A1; WO 2013/174895A1; WO 2014/074660; WO 2014/074661; WO 2014/074670; WO 2015/032423; WO 2015/069310; WO 2015/089143; WO 2015/091584A1; WO 2015/131080A1; WO 2016/138352A1; US 2017/0240552; WO 2017/040757A1; WO 2017/087590; WO 2018/067432; WO 2018/071794A1; WO 2018/075937A1; WO 2019/081488; WO 2018/093968; WO 2018/111787; WO 2018/165240A1; WO 2019/023468A1; WO 2019/183186; US 2020/0231594; WO 2020/074461A1; WO 2020/081508A1; WO 2020/086616A1; WO 2020/092196; WO 2020/112937A1; WO 2020/154474A1; WO 2020/159904; WO 2020/163778A1; WO 2020/185755A1; WO 2020/198379A1; WO 2020/222773A1; WO 2020/223431A1; WO 2021/048618A1; WO 2021/048620A1; WO 2021/092246A1; WO 2021/055651A1; WO 2021/055652A1; WO 2021/180072A1; WO 2022/166917; CN114805438; WO 2022/165141; WO 2022/156657; WO 2022/150446; WO 2022/135430; WO 2022/127869; WO 2022/121868; WO 2022/117090; WO 2022/105771; WO 2022/109492; WO 2022/099431; WO 2022/060973; CN114181199; WO 2022/032484; CN114057651; CN 113773262; WO 2021/259208; US 2022/002267; US 2022/0009910; WO 2022/011337; WO 2022/011338; WO 2022/017494; CN113968846; CN 113698403; CN 113563309; WO 2020/207476; WO 2020/154474; WO 2020/185755; CN112592345; WO 2021/198379; WO 2021/170046; WO 2021/204626; WO 2021/092246; CN111909140; WO 2021/027647; CN 113735836; CN113735837; CN 113735859; WO 2021/202652; CN110818641; WO 2021/237121; WO 2021/222153; CN113666877; CN113480543; CN112142675; WO 2020/112937; WO 2021/211741; CN112159394; CN111961037; WO 2020/259584; WO 2020/163778; WO 2020/081508; WO 2020/074461; WO 2016/047678; WO 2020/156311; WO 2020/038457; WO 2020/222773; WO 2020/223431; U.S. Pat. No. 10,517,876; JP2016065023; WO 2021/078020; WO 2021/078023; WO 2021/078022; WO 2020/086616; DE102009001438; DE102009015070; WO 2015/091584; U.S. Pat. No. 10,308,646; WO 2019/023468; WO 2019/023468; WO 2013/174895; US 2021/087154; WO 2013/125543; WO 2018/071794; WO 2013/146963; WO 2015/016206; WO 2018/075937; WO 2017/040757; WO 2012/000970; WO 2016/027195; WO 2016/138352; WO 2015/131080; WO 2011/113802; WO 2012/062704; WO 2007/070514; WO 2022/213980; WO 2022/206705; WO 2022/193499; WO 2022/188796; WO 2022/175745; WO 2022/175746; WO 2022/175747; WO 2022/175752; and CN114907326, the entirety of each is herein incorporated by reference.

In some embodiments, the compounds of the invention have a structure of Formula (IIAA′):

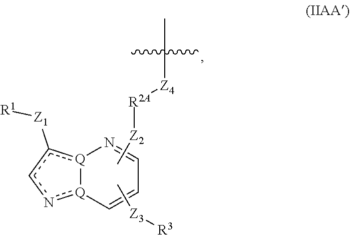

or a pharmaceutically acceptable salt thereof,
wherein Q is independently at each occurrence selected from —CH— and —N— when Q is attached to only single bonds, or Q is —C═ when Q is attached to a double bond;
R¹ is selected from a hydrogen, a C₁-C₆ aliphatic, Ring 1, —C₁-C₆ alkylene-Ring 1, and -Ring 1′-C₁-C₆ aliphatic; wherein each of the C₁-C₆ aliphatic, the Ring 1, the C₁-C₆ alkylene-Ring 1, and the -Ring 1′-C₁-C₆ aliphatic is independently optionally substituted with one or more of —F, —Cl, —Br, —I, and —OR^{C};
R^{2A} is selected from a covalent bond, a C₁-C₁₂ alkylene, which C₁-C₁₂ alkylene optionally comprises 1-8 heteroatoms independently selected from O, N, and S, and Ring 2A, or R^{2A} is —C₁-C₁₂ alkylene-Ring 2A-, -Ring 2A-Z₅-, or -(Ring 2A)-Z₅-(Ring 2A)-, wherein when R^{2A} is -(Ring 2A)-Z₅-(Ring 2A)- two Ring 2A may be the same or different; wherein each of the C₁-C₁₂ alkylene and the Ring 2A is independently optionally substituted with one or more of R^{K};
R³ is selected from a hydrogen and a C₁-C₆ aliphatic, which C₁-C₆ aliphatic optionally comprises 1-8 heteroatoms independently selected from O, N, and S, wherein the C₁-C₆ aliphatic is optionally substituted with one or more of R^{K};
Z₁ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₂ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₃ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₄ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₅ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S;
Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S;
Ring 2A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
each R^{K} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, a C₁-C₆ aliphatic group, or two R^{K} groups attached to the same carbon or sulfur atom are optionally taken together to form ═O;
each R^{C} is independently hydrogen or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S,
and

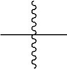

represents the point of attachment to L.

In some embodiments, the compounds of the invention have a structure of Formula (IIA-L-DIM) or Formula (IIB-L-DIM):

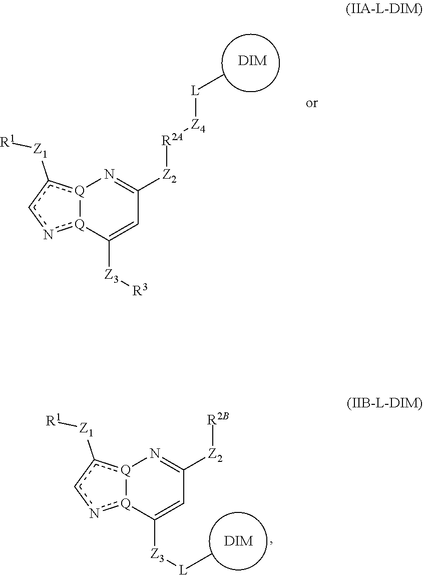

or a pharmaceutically acceptable salt thereof, wherein Q, Z₁, Z₂, Z₃, Z₄, R¹, R^{2A}, R^{2B}, R³, L, and DIM are as defined below.

In some embodiments, TBM has a structure of Formula (IIA′), Formula (IIA″), or Formula (IIB):

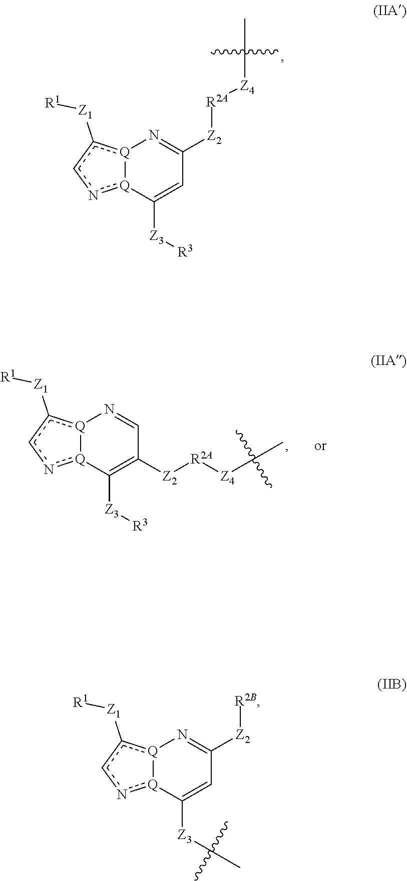

or a pharmaceutically acceptable salt thereof,
wherein Q is independently at each occurrence selected from —CH— and —N— when Q is attached to only single bonds, or Q is —C═ when Q is attached to a double bond;
R¹ is selected from a hydrogen, a C₁-C₆ aliphatic, Ring 1, —C₁-C₆ alkylene-Ring 1, and -Ring 1′-C₁-C₆ aliphatic; wherein each of the C₁-C₆ aliphatic, the C₁-C₆ alkylene, the Ring 1, the —C₁-C₆ alkylene-Ring 1, and the -Ring 1′-C₁-C₆ aliphatic is independently optionally substituted with one or more of —F, —Cl, —Br, —I, and —OR^{C};
R^{2A} is selected from a covalent bond, a C₁-C₁₂ alkylene, which C₁-C₁₂ alkylene optionally comprises 1-8 heteroatoms independently selected from O, N, and S, and Ring 2A, or R^{2A} is —C₁-C₁₂ alkylene-Ring 2A-, -Ring 2A-Z₅-, or -(Ring 2A)-Z₅-(Ring 2A)-, wherein when R^{2A} is -(Ring 2A)-Z₅-(Ring 2A)- two Ring 2A may be the same or different; wherein each of the C₁-C₁₂ alkylene and the Ring 2A is independently optionally substituted with one or more of R^{K};
R^{2B} is selected from a hydrogen, a C₁-C₆ aliphatic, and Ring 2B, or R^{2B} is —C₁-C₆ alkylene-Ring 2B, -Ring 2B′-C₁-C₆ aliphatic, -Ring 2B′-Z₅-Ring 2B wherein each of the C₁-C₆ aliphatic, the C₁-C₆ alkylene, the Ring 2B, the Ring 2B′ is independently optionally substituted with one or more of R^{K};
R³ is selected from a hydrogen and a C₁-C₆ aliphatic, which C₁-C₆ aliphatic optionally comprises 1-8 heteroatoms independently selected from O, N, and S, wherein the C₁-C₆ aliphatic is optionally substituted with one or more of R^{K};
Z₁ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₂ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₃ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₄ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₅ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S;
Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S;
Ring 2A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
Ring 2B is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S; and
Ring 2B′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
each R^{K} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, a C₁-C₆ aliphatic group, or two R^{K} groups attached to the same carbon or sulfur atom are optionally taken together to form ═O;
each R^{C} is independently hydrogen or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S,
and

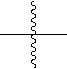

represents the point of attachment to L.

In some embodiments, TBM has a structure of Formula (IIA-1):

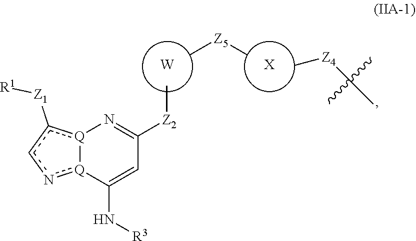

or a pharmaceutically acceptable salt thereof,
wherein
Q is independently at each occurrence selected from —CH— and —N— when Q is attached to only single bonds, or Q is —C═ when Q is attached to a double bond;
R¹ is -Cy-H optionally substituted with one or more of R*, and combinations thereof;
Ring W is a ring selected from phenylenyl, a 4 to 7-membered saturated or partially unsaturated carbocyclylenyl or heterocyclylenyl with 1-3 heteroatoms independently selected from N, O, and S, and a 5 to 6-membered heteroarylenyl with 1-4 heteroatoms independently selected from N, O, and S, and each of which further comprises 0-8 heteroatoms selected from halogen, O, N, and S, and optionally substituted with one or more of —F, —Cl, —Br, —I, —OH, —OR*, —NO, —NO₂, —NO₃, —O—NO, —N₃, —NH₂, —NHR*, —N(R*)₂, —N(R*)₃⁺, —N(R*)—OH, —O—N(R*)₂, —N(R*)—O—R*, —CN, —NC, —C(O)—R*, —CHO, —CO₂H, —CO₂R*, —C(O)—S—R*, —O—C(O)—H, —O—C(O)—R*, —S—C(O)—R*, —C(O)—NH₂, —C(O)—N(R*)₂, —C(O)—NHNH₂, —O—C(O)—NHNH₂, —C(S)—NH₂, —C(S)—N(R*)₂, —N(R*)—CHO, —N(R*)—C(O)—R*, —SCN, —NCS, —NSO, —SSR*, —SO₂R*, —SO₂—N(R*)₂, —S(O)—OR*, —S(O)—R*, —Si(R*)₃, —CF₃, —O—CF₃ and combinations thereof;
Ring X is absent, or a ring selected from phenylenyl, a 4 to 7-membered saturated or partially unsaturated carbocyclylenyl or heterocyclylenyl with 1-3 heteroatoms independently selected from N, O, and S, and a 5 to 6-membered heteroarylenyl with 1-4 heteroatoms independently selected from N, O, and S, and each of which further comprises 0-8 heteroatoms selected from halogen, O, N, and S, and optionally substituted with one or more of —F, —Cl, —Br, —I, —OH, —OR*, —NO, —NO₂, —NO₃, —O—NO, —N₃, —NH₂, —NHR*, —N(R*)₂, —N(R*)₃⁺, —N(R*)—OH, —O—N(R*)₂, —N(R*)—O—R*, —CN, —NC, —C(O)—R*, —CHO, —CO₂H, —CO₂R*, —C(O)—S—R*, —O—C(O)—H, —O—C(O)—R*, —S—C(O)—R*, —C(O)—NH₂, —C(O)—N(R*)₂, —C(O)—NHNH₂, —O—C(O)—NHNH₂, —C(S)—NH₂, —C(S)—N(R*)₂, —N(R*)—CHO, —N(R*)—C(O)—R*, —SCN, —NCS, —NSO, —SSR*, —SO₂R*, —SO₂—N(R*)₂, —S(O)—OR*, —S(O)—R*, —Si(R*)₃, —CF₃, —O—CF₃ and combinations thereof;
R³ is an aliphatic C₁-C₁₂ hydrocarbon optionally comprising 0-8 heteroatoms selected from halogen, O, N, and S;
Z₁ is —C(O)N(R*)—;
Z₂ is selected from a covalent bond and —NR*—;
Z₄ is selected from a covalent bond, —C(O)O—, and —C(O)N(R*)—,
Z₅ is selected from a covalent bond, —O—, —NR*—, —S—, C(R*)₂—, —C(O)—, C(O)O—, and —C(O)N(R*)—,
each R* is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, or:
two R* groups on the same nitrogen are optionally taken together with their intervening atoms to form a 4 to 7-membered saturated, partially unsaturated, or heteroaryl ring having 0-3 heteroatoms in addition to the nitrogen, independently selected from N, O, and S,
and

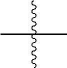

represents the point of attachment to the linker L.

In some embodiments, TBM has a structure of Formula (IIA′-1) or Formula (IIA″-1):

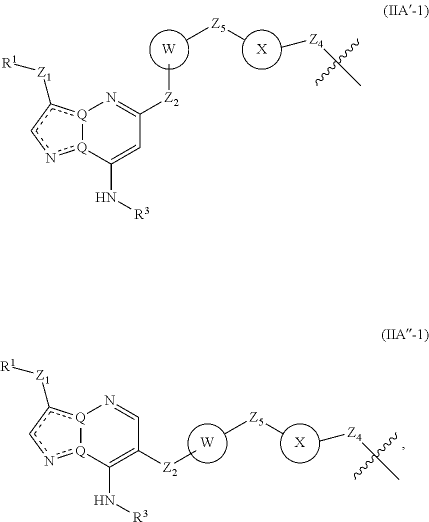

or a pharmaceutically acceptable salt thereof,
wherein Q, R¹, Ring 1, Ring 1′, R³, R^{k}, R^{c}, and

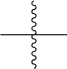

are defined above,
Ring W is a ring selected from phenylenyl, a 4 to 7-membered saturated or partially unsaturated carbocyclylenyl or heterocyclylenyl with 1-3 heteroatoms independently selected from N, O, and S, and a 5 to 6-membered heteroarylenyl with 1-4 heteroatoms independently selected from N, O, and S, and each of which is optionally substituted with one or more of R^{K};
Ring X is absent, or a ring selected from phenylenyl, a 4 to 7-membered saturated or partially unsaturated carbocyclylenyl or heterocyclylenyl with 1-3 heteroatoms independently selected from N, O, and S, and a 5 to 6-membered heteroarylenyl with 1-4 heteroatoms independently selected from N, O, and S, and the ring is optionally substituted with one or more of R^{K};
Z₁ is —C(O)N(R^{C})—;
Z₂ is selected from a covalent bond and —NR^{C}—;
Z₄ is selected from a covalent bond, —C(O)O—, and —C(O)N(R^{C})—,
Z₅ is selected from a covalent bond, —O—, —NR^{C}—, —S—, C(R^{C})₂—, —C(O)—, C(O)O—, and —C(O)N(R^{C})—.

In some embodiments, TBM has a structure selected from Formula (IIA-2) and Formula (IIA-3):

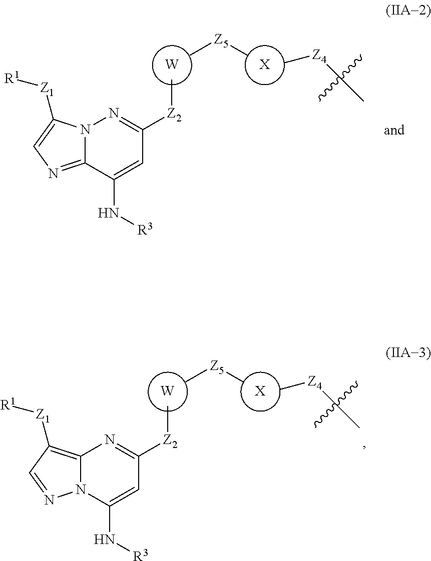

or a pharmaceutically acceptable salt thereof.

In some embodiments, TBM has a structure of Formula (IIB-1):

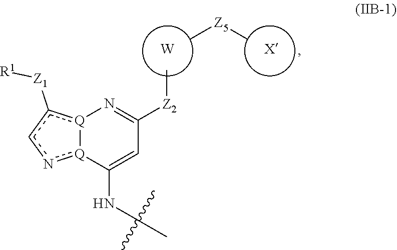

or a pharmaceutically acceptable salt thereof, wherein Q is as defined above;
R¹ is -Cy-H optionally substituted with one or more of —F, —Cl, —Br, —I, —OH, —N₃, —NH₂, —CF₃, and combinations thereof;
Ring W is a ring selected from phenylenyl, a 4 to 7-membered saturated or partially unsaturated carbocyclyl or heterocyclylenyl with 1-3 heteroatoms independently selected from N, O, and S, and a 5 to 6-membered heteroarylenyl with 1-4 heteroatoms independently selected from N, O, and S, and each of which further comprises 0-8 heteroatoms selected from halogen, O, N, and S, and optionally substituted with one or more of —F, —Cl, —Br, —I, —OH, —OR*, —NO, —NO₂, —NO₃, —O—NO, —N₃, —NH₂, —NHR*, —N(R*)₂, —N(R*)₃⁺, —N(R*)—OH, —O—N(R*)₂, —N(R*)—O—R*, —CN, —NC, —C(O)—R*, —CHO, —CO₂H, —CO₂R*, —C(O)—S—R*, —O—C(O)—H, —O—C(O)—R*, —S—C(O)—R*, —C(O)—NH₂, —C(O)—N(R*)₂, —C(O)—NHNH₂, —O—C(O)—NHNH₂, —C(S)—NH₂, —C(S)—N(R*)₂, —N(R*)—CHO, —N(R*)—C(O)—R*, —SCN, —NCS, —NSO, —SSR*, —SO₂R*, —SO₂—N(R*)₂, —S(O)—OR*, —S(O)—R*, —Si(R*)₃, —CF₃, —O—CF₃ and combinations thereof;
Ring X′ is absent, or a ring selected from phenyl, a 4 to 7-membered saturated or partially unsaturated carbocyclyl or heterocyclyl with 1-3 heteroatoms independently selected from N, O, and S, and a 5 to 6-membered heteroaryl with 1-4 heteroatoms independently selected from N, O, and S, and each of which further comprises 0-8 heteroatoms selected from halogen, O, N, and S, and optionally substituted with one or more of —F, —Cl, —Br, —I, —OH, —OR*, —NO, —NO₂, —NO₃, —O—NO, —N₃, —NH₂, —NHR*, —N(R*)₂, —N(R*)₃⁺, —N(R*)—OH, —O—N(R*)₂, —N(R*)—O—R*, —CN, —NC, —C(O)—R*, —CHO, —CO₂H, —CO₂R*, —C(O)—S—R*, —O—C(O)—H, —O—C(O)—R*, —S—C(O)—R*, —C(O)—NH₂, —C(O)—N(R*)₂, —C(O)—NHNH₂, —O—C(O)—NHNH₂, —C(S)—NH₂, —C(S)—N(R*)₂, —N(R*)—CHO, —N(R*)—C(O)—R*, —SCN, —NCS, —NSO, —SSR*, —SO₂R*, —SO₂—N(R*)₂, —S(O)—OR*, —S(O)—R*, —Si(R*)₃, —CF₃, —O—CF₃ and combinations thereof;
Z₁ is —C(O)N(R*)—;
Z₂ is selected from a covalent bond and —NR*—;
Z₅ is selected from a covalent bond, —O—, —NR*—, —S—, C(R*)₂—, —C(O)—, C(O)O—, and —C(O)N(R*)—,
each R* is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, or:
two R* groups on the same nitrogen are optionally taken together with their intervening atoms to form a 4 to 7-membered saturated, partially unsaturated, or heteroaryl ring having 0-3 heteroatoms in addition to the nitrogen, independently selected from N, O, and S,
and

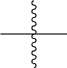

represents the point of attachment to L.

In some embodiments, TBM has a structure selected from Formula (IIA′-2) to Formula (IIA′-7) and Formula (IIA″-2) to Formula (IIA″-7):

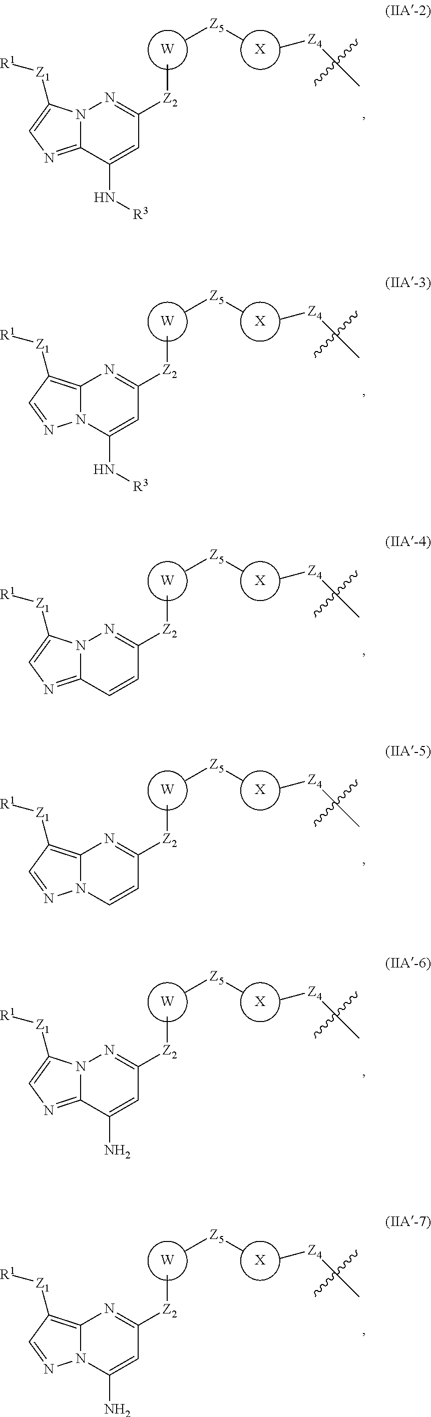

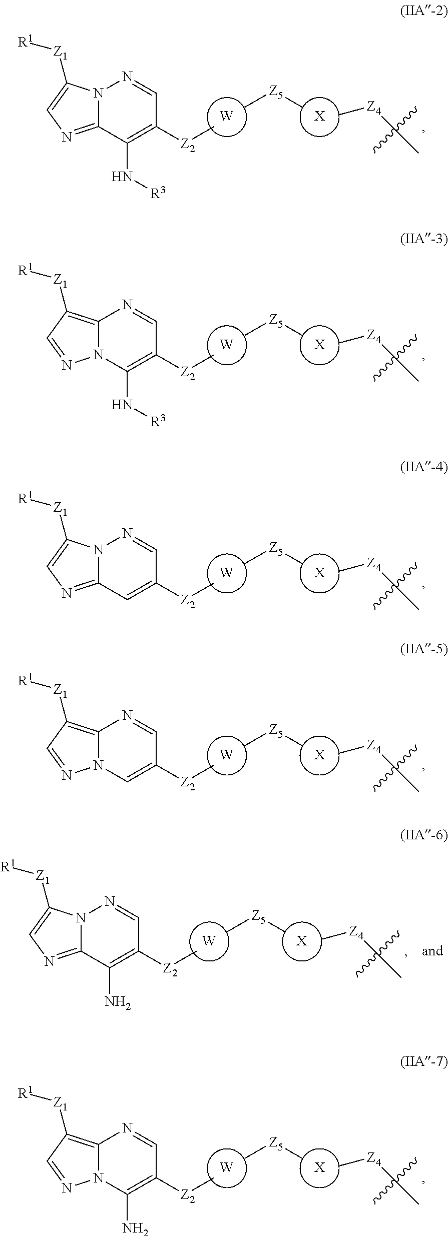

or a pharmaceutically acceptable salt thereof, wherein
R¹, R³, Z₁, Z₂, Z₄, Z₅, Ring 1, Ring 1′, R^{k}, R^{c}, and

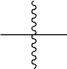

are defined above,
Ring W is a ring selected from phenylenyl, a 4 to 7-membered saturated or partially unsaturated carbocyclylenyl or heterocyclylenyl with 1-3 heteroatoms independently selected from N, O, and S, and a 5 to 6-membered heteroarylenyl with 1-4 heteroatoms independently selected from N, O, and S, and each of which is optionally substituted with one or more of R^{K}; and
Ring X is absent, or a ring selected from phenylenyl, a 4 to 7-membered saturated or partially unsaturated carbocyclylenyl or heterocyclylenyl with 1-3 heteroatoms independently selected from N, O, and S, and a 5 to 6-membered heteroarylenyl with 1-4 heteroatoms independently selected from N, O, and S, and the ring is optionally substituted with one or more of R^{K}.

In some embodiments, TBM has a structure of Formula (IIB-1):

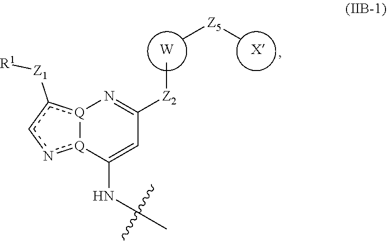

or a pharmaceutically acceptable salt thereof,
wherein Q, R¹, Ring 1, Ring 1′, R^{k}, R^{c}, and

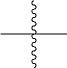

are defined above;
Ring W is absent or a ring selected from phenylenyl, a 4 to 7-membered saturated or partially unsaturated carbocyclylenyl, a 4 to 7-membered saturated or partially unsaturated heterocyclylenyl with 1-3 heteroatoms independently selected from N, O, and S, and a 5 to 6-membered heteroarylenyl with 1-4 heteroatoms independently selected from N, O, and S, and the ring is optionally substituted with one or more of R^{K};
Ring X′ is a ring selected from phenyl, a 4 to 7-membered saturated or partially unsaturated carbocyclyl or heterocyclyl with 1-3 heteroatoms independently selected from N, O, and S, and a 5 to 6-membered heteroaryl with 1-4 heteroatoms independently selected from N, O, and S, and each of which is optionally substituted with one or more of R^{K};
Z₁ is —C(O)N(R^{C})—;
Z₂ is selected from a covalent bond and —NR^{C}—; and
Z₅ is selected from a covalent bond, —O—, —NR^{C}—, —S—, C(R^{C})₂—, —C(O)—, C(O)O—, and —C(O)N(R^{C})—;

In some embodiments, TBM has a structure selected from Formula (IIA-2′) and Formula (IIA-3′):

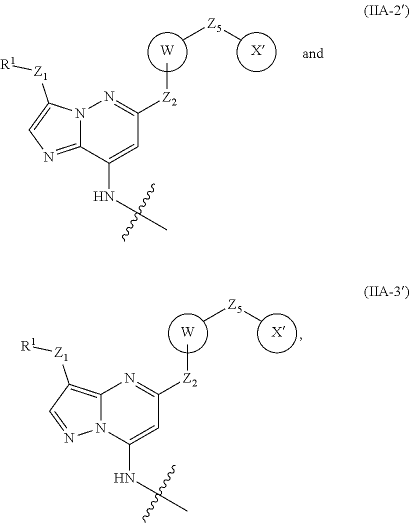

or a pharmaceutically acceptable salt thereof.

As defined above and described herein, Ring W is selected from phenylenyl, a 4-7 membered saturated or partially unsaturated heterocyclylenyl having 1-2 heteroatoms independently selected from N, O, and S, a 5-6 membered heteroarylenyl having 1-4 heteroatoms independently selected from N, O, and S and combinations thereof.

In some embodiments, Ring W is a ring selected from phenylenyl, a 5 to 6-membered saturated or partially unsaturated carbocyclylenyl or heterocyclylenyl with 1-3 heteroatoms independently selected from N, O, and S, and a 5 to 6-membered heteroarylenyl with 1-4 heteroatoms independently selected from N, O, and S, and each of which is optionally substituted with one or more of R^{K}.

As defined above and described herein, Ring X is absent, or a ring selected from phenylenyl, a 4-7 membered saturated or partially unsaturated heterocyclylenyl having 1-2 heteroatoms independently selected from N, O, and S, a 5-6 membered heteroarylenyl having 1-4 heteroatoms independently selected from N, O, and S and combinations thereof.

In some embodiments, Ring X is absent or a ring selected from phenylenyl, a 4 to 6-membered saturated or partially unsaturated carbocyclylenyl or heterocyclylenyl with 1-3 heteroatoms independently selected from N, O, and S, and a 5 to 6-membered heteroarylenyl with 1-4 heteroatoms independently selected from N, O, and S, and each of which is optionally substituted with one or more of R^{K}.

As defined above and described herein, Ring X′ is a ring selected from phenyl, a 4 to 6-membered saturated or partially unsaturated carbocyclyl or heterocyclyl with 1-3 heteroatoms independently selected from N, O, and S, and a 5 to 6-membered heteroaryl with 1-4 heteroatoms independently selected from N, O, and S, and each of which is optionally substituted with one or more of R^{K}.

As defined above and described herein, R^{2B} is is selected from a hydrogen, an aliphatic C₁-C₂₀ hydrocarbon, -Cy-H, a C₁-C₂₀ alkylene-Cy-H, -Cy-C₁-C₂₀ aliphatic, wherein when R¹ is non hydrogen, R¹ further comprises 0-8 heteroatoms selected from halogen, O, N, and S, and R¹ is optionally substituted with one or more of —F, —Cl, —Br, —I, —OH, —OR*, —NO, —NO₂, —NO₃, —O—NO, —N₃, —NH₂, —NHR*, —N(R*)₂, —N(R*)₃⁺, —N(R*)—OH, —O—N(R*)₂, —N(R*)—O—R*, —CN, —NC, —C(O)—R*, —CHO, —CO₂H, —CO₂R*, —C(O)—S—R*, —O—C(O)—H, —O—C(O)—R*, —S—C(O)—R*, —C(O)—NH₂, —C(O)—N(R*)₂, —C(O)—NHNH₂, —O—C(O)—NHNH₂, —C(S)—NH₂, —C(S)—N(R*)₂, —N(R*)—CHO, —N(R*)—C(O)—R*, —SCN, —NCS, —NSO, —SSR*, —SO₂R*, —SO₂—N(R*)₂, —S(O)—OR*, —S(O)—R*, —Si(R*)₃, —CF₃, —O—CF₃, wherein:

-Cy- is an optionally substituted ring selected from phenylenyl, an 8-10 membered bicyclic arylenyl, a 3-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 4-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, and the combinations thereof,
each R* is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, or:
two R* groups on the same nitrogen are optionally taken together with their intervening atoms to form a 4 to 7-membered saturated, partially unsaturated, or heteroaryl ring having 0-3 heteroatoms in addition to the nitrogen, independently selected from N, O, and S.

In some embodiments, R^{2B} is selected from a hydrogen, a C₁-C₆ aliphatic, and Ring 2B, wherein the C₁-C₆ aliphatic and the Ring 2B is optionally substituted with one or more of R^{K}.

As defined above and described herein, Ring 2B is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S.

In some embodiments, R^{2B} is

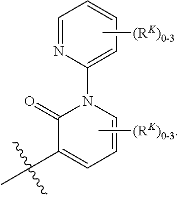

In some embodiments, R^{2B} is

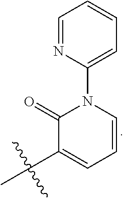 [SMILES: *c1cccn(-c2ccccn2)c1=O]

As defined above and described herein, -Z₂-R^{2B} is selected from:

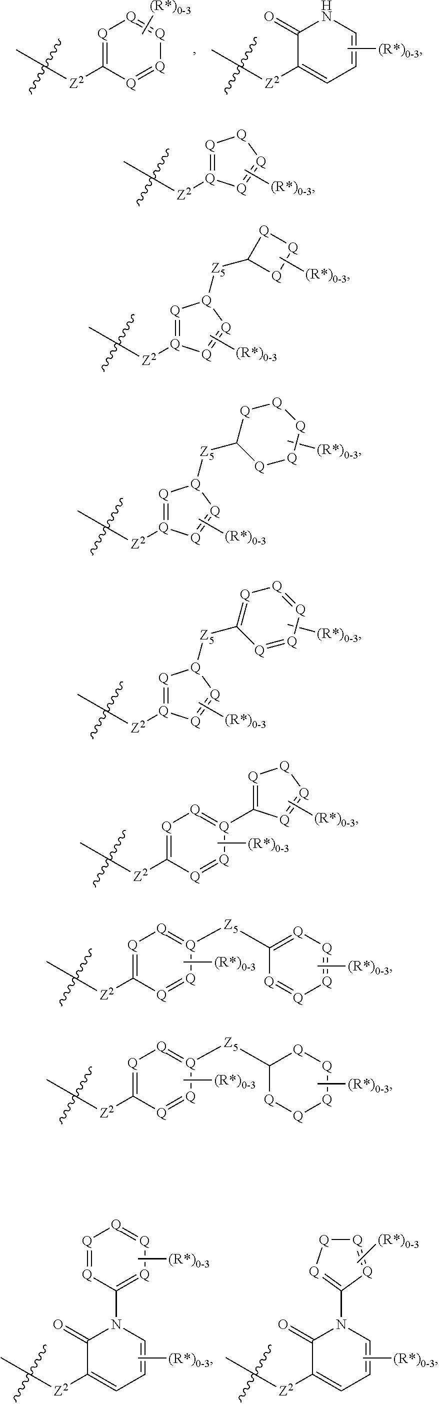

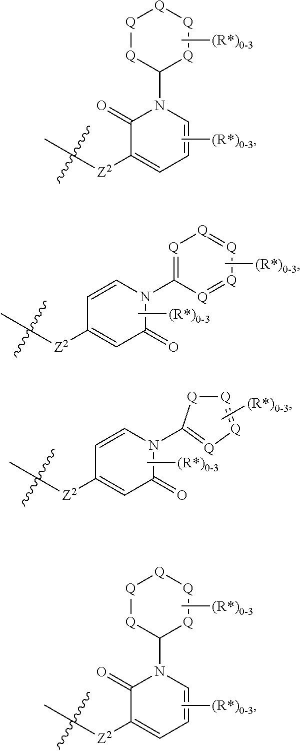

wherein Q is independently at each occurrence —C—, —N—, or —O—, and R* is independently at each occurrence hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, and combinations thereof.

In some embodiments, -Z₂-R^{2B} is selected from:

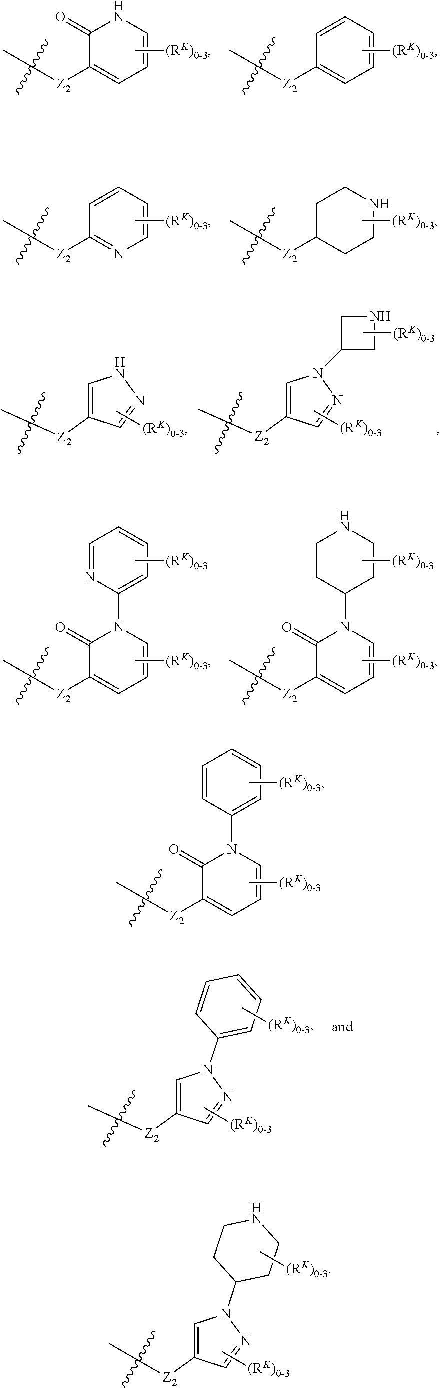

As defined above and described herein, R¹ is selected from a hydrogen, an aliphatic C₁-C₂₀ hydrocarbon, -Cy-H, a C₁-C₂₀ alkylene-Cy-H, -Cy-C₁-C₂₀ aliphatic, wherein when R¹ is non hydrogen, R¹ further comprises 0-8 heteroatoms selected from halogen, O, N, and S, and wherein R¹ is optionally substituted with one or more of —F, —Cl, —Br, —I, —OH, —OR*, —NO, —NO₂, —NO₃, —O—NO, —N₃, —NH₂, —NHR*, —N(R*)₂, —N(R*)₃⁺, —N(R*)—OH, —O—N(R*)₂, —N(R*)—O—R*, —CN, —NC, —C(O)—R*, —CHO, —CO₂H, —CO₂R*, —C(O)—S—R*, —O—C(O)—H, —O—C(O)—R*, —S—C(O)—R*, —C(O)—NH₂, —C(O)—N(R*)₂, —C(O)—NHNH₂, —O—C(O)—NHNH₂, —C(S)—NH₂, —C(S)—N(R*)₂, —N(R*)—CHO, —N(R*)—C(O)—R*, —SCN, —NCS, —NSO, —SSR*, —SO₂R*, —SO₂—N(R*)₂, —S(O)—OR*, —S(O)—R*, —Si(R*)₃, —CF₃, —O—CF₃, wherein:

-Cy- is an optionally substituted ring selected from phenylenyl, an 8-10 membered bicyclic arylenyl, a 3-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 4-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, and the combinations thereof
each R* is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, or:
two R* groups on the same nitrogen are optionally taken together with their intervening atoms to form a 4 to 7-membered saturated, partially unsaturated, or heteroaryl ring having 0-3 heteroatoms in addition to the nitrogen, independently selected from N, O, and S,

In some embodiments, R¹ is selected from a hydrogen, Ring 1, -Ring 1′-C₁-C₆ aliphatic, wherein the Ring 1 or the -Ring 1′-C₁-C₆ aliphatic is optionally substituted with one or more of —F, —Cl, —Br, —I, and —OR^{C}.

In some embodiments, R¹ is a 3-7 membered saturated or partially unsaturated carbocyclylenyl substituted with one or more of —F, —Cl, —Br, —I, —OH, —N₃, —NH₂, —CF₃.

In some embodiments, R¹ is selected from hydrogen,

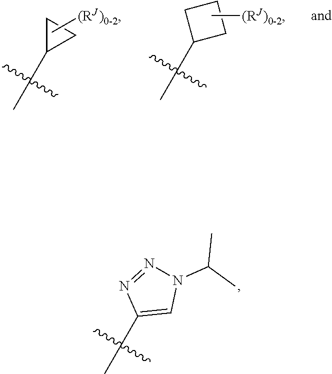

wherein R^{J} is selected from —F, —Cl, —Br, —I, and OR^{C}.

In some embodiments, R^{J} is —F, —Cl, —Br, —I, —OH, —NH₂, or a C₁-C₆ aliphatic.

In some embodiments, R¹ is

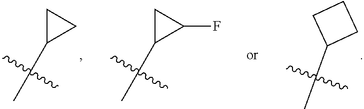

In some embodiments, R¹ is

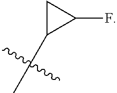 [SMILES: *C1CC1F]

In some embodiments, R¹ is

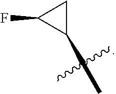 [SMILES: *[C@@H]1C[C@@H]1F]

As defined above and described herein, -Z₁-R¹ is selected from hydrogen,

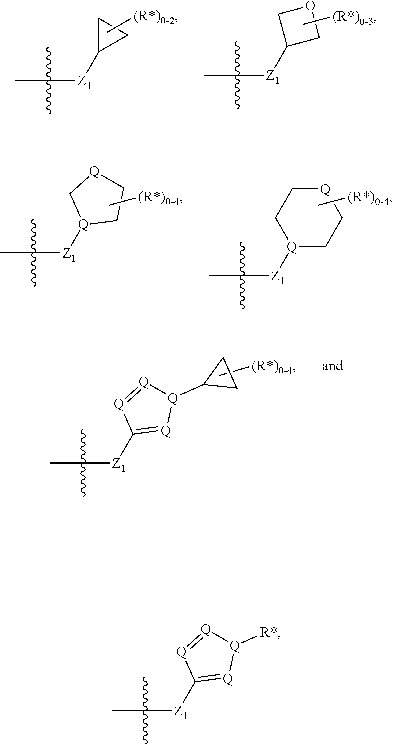

wherein Q is independently at each occurrence —C—, —N—, or —O—, and R* is independently at each occurrence hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, and combinations thereof.

In some embodiments, -Z₁-R¹ is selected from hydrogen,

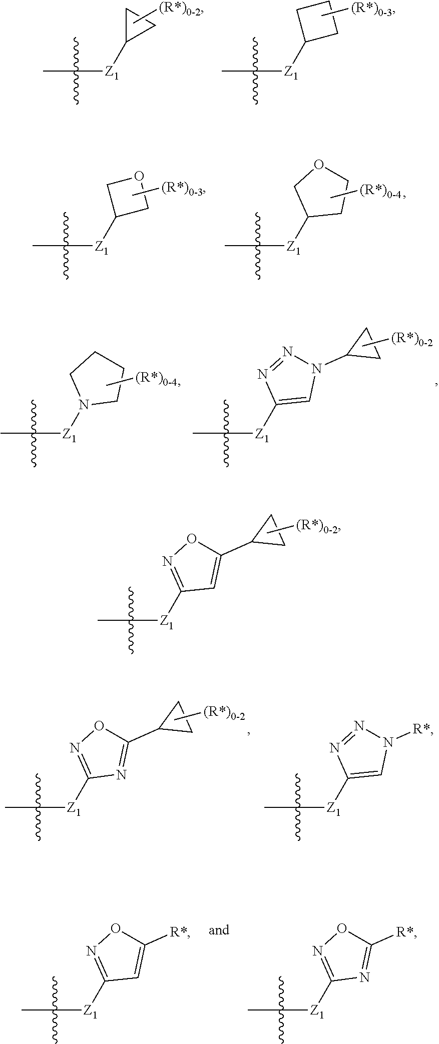

wherein R* is independently at each occurrence hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, and combinations thereof.

In some embodiments, -Z₁-R¹ is

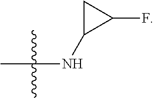 [SMILES: *NC1CC1F]

In some embodiments, -Z₁-R¹ is

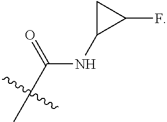 [SMILES: *C(=O)NC1CC1F]

In some embodiments, -Z₁-R¹ is

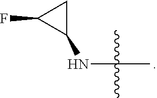 [SMILES: *N[C@@H]1C[C@@H]1F]

In some embodiments, -Z₁-R¹ is

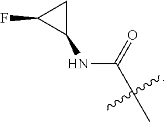 [SMILES: *C(=O)N[C@@H]1C[C@@H]1F]

As defined above and described herein, R^{2A} is -Ring 2A-Z₅- or R^{2A} is -(Ring 2A)-Z₅-(Ring 2A)-, wherein when R^{2A} is -(Ring 2A)-Z₅-(Ring 2A)-, two Ring 2A may be same or different, wherein each Ring 2A is independently an optionally substituted ring selected from phenylenyl, a 3-7 membered saturated or partially unsaturated carbocyclylenyl, a 4-7 membered saturated or partially unsaturated heterocyclylenyl having 1-3 heteroatoms independently selected from N, O, and S, and a 5-6 membered heteroarylenyl having 1-4 heteroatoms independently selected from N, O, and S, and wherein the Ring 2A is independently optionally substituted with one or more of R^{K}.

In some embodiments, R^{2A} is selected from a covalent bond, a C₁-C₁₂ alkylene, which C₁-C₁₂ alkylene optionally comprises 1-8 heteroatoms independently selected from O, N, and S, and Ring 2A, wherein each of the C₁-C₁₂ alkylene and the Ring 2A is optionally substituted with one or more of R^{K}.

In some embodiments, R^{2A} is a hydrogen, a C₁-C₂₀ alkylene, -Cy-, and combinations of C₁-C₂₀ alkylene and -Cy-, wherein R^{2A} is non hydrogen, R^{2A} further comprises 0-8 heteroatoms selected from halogen, O, N, and S, and wherein R^{2A} is optionally substituted with one or more of —F, —Cl, —Br, —I, —OH, —OR*, —NO, —NO₂, —NO₃, —O—NO, —N₃, —NH₂, —NHR*, —N(R*)₂, —N(R*)₃⁺, —N(R*)—OH, —O—N(R*)₂, —N(R*)—O—R*, —CN, —NC, —C(O)—R*, —CHO, —CO₂H, —CO₂R*, —C(O)—S—R*, —O—C(O)—H, —O—C(O)—R*, —S—C(O)—R*, —C(O)—NH₂, —C(O)—N(R*)₂, —C(O)—NHNH₂, —O—C(O)—NHNH₂, —C(S)—NH₂, —C(S)—N(R*)₂, —N(R*)—CHO, —N(R*)—C(O)—R*, —SCN, —NCS, —NSO, —SSR*, —SO₂R*, —SO₂—N(R*)₂, —S(O)—OR*, —S(O)—R*, —Si(R*)₃, —CF₃, —O—CF₃ and combinations thereof, wherein:
-Cy- is an optionally substituted ring selected from phenylenyl, an 8-10 membered bicyclic arylenyl, a 3-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 4-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, and
R* is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, or:
two R* groups on the same nitrogen are optionally taken together with their intervening atoms to form a 4 to 7-membered saturated, partially unsaturated, or heteroaryl ring having 0-3 heteroatoms in addition to the nitrogen, independently selected from N, O, and S,

As defined above and described herein, -Z₂-R^{2A}-Z₄- is selected from:

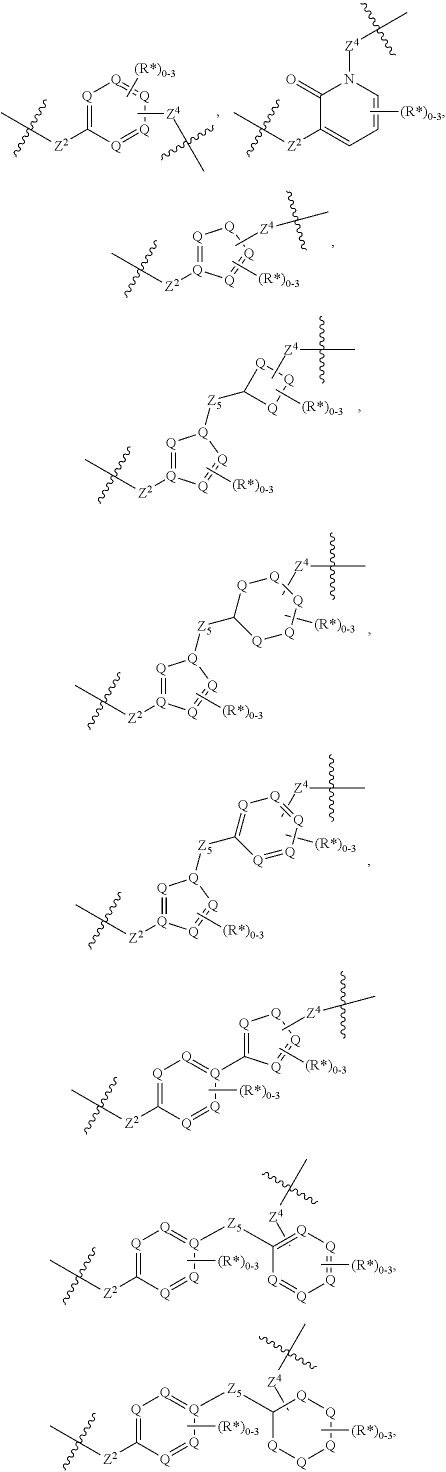

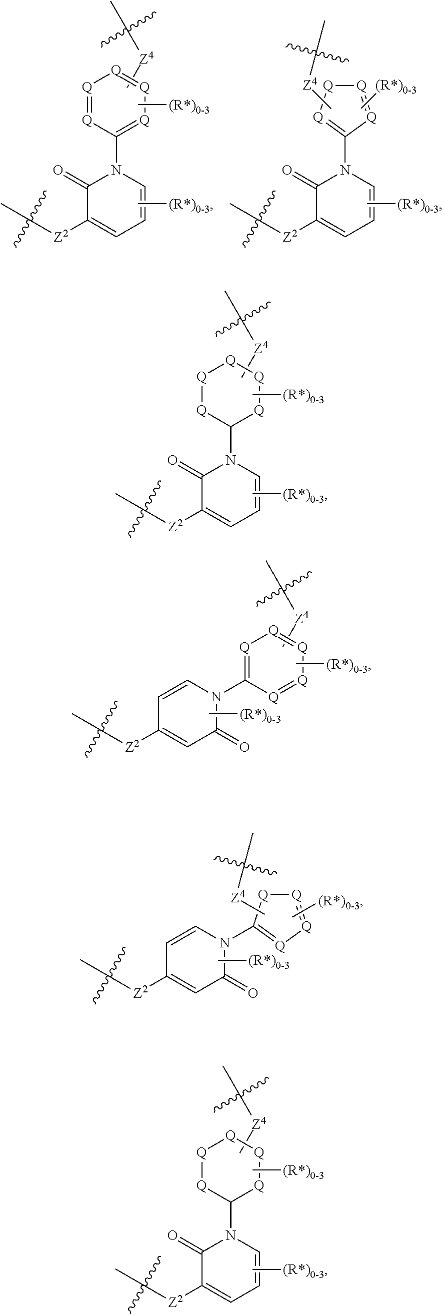

wherein Q is independently at each occurrence —C—, —N—, or —O—, and R* is independently at each occurrence hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁₋₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, and combinations thereof.

In some embodiments, -Z₂-R^{2A}-Z₄- is selected from:

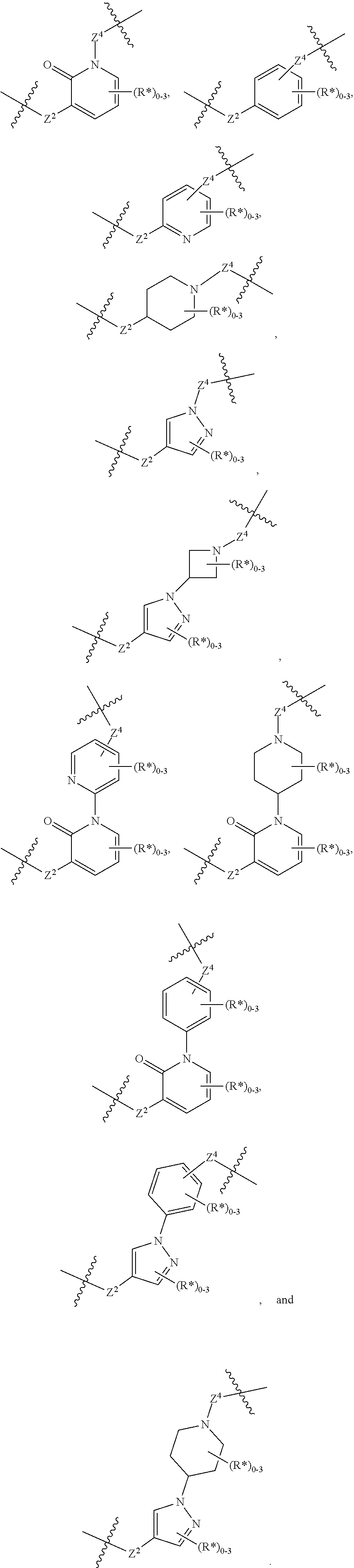

In some embodiments, -Z₂-R^{2A}-Z₄- is selected from

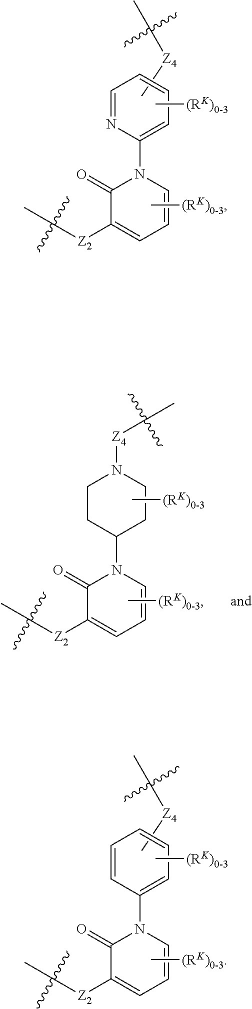

In some embodiments, -Z₂-R^{2A}-Z₄- is selected from

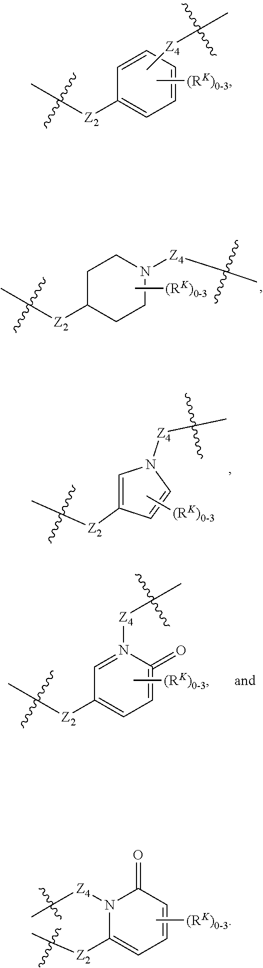

In some embodiments, -Z₂-R^{2A}-Z₄- is selected from

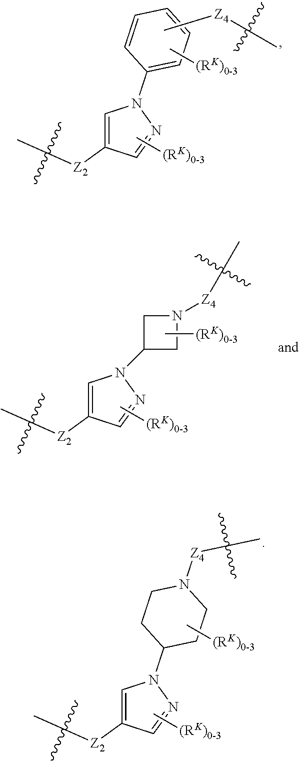

As defined above and described herein, R³ is selected from a hydrogen and a C₁-C₃ aliphatic, the C₁-C₃ aliphatic optionally comprises 1-8 heteroatoms independently selected from O, N, and S, and the C₁-C₃ aliphatic is optionally substituted with one or more of R^{K}.

In some embodiments, R³ is methyl.

As defined above and described herein, Z₁ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—.

In some embodiments, Z₁ is selected from a covalent bond, —O—, —NR^{C}—, —C(O)—, —CR^{C}₂—, —OC(O)—, —C(O)O—, —N(R^{C})C(O)—, and —C(O)N(R^{C})—.

In some embodiments, Z₁ is —C(O)N(R^{C})—.

In some embodiments, Z₁ is a covalent bond or —C(O)NH—.

As defined above and described herein, Z₂ is selected from a covalent bond, —O—, —NR^{C}—, —C(O)—, —CR^{C}₂—, —OC(O)—, —C(O)O—, —N(R^{C})C(O)—, and —C(O)N(R^{C})—.

In some embodiments, Z₂ is —NR^{c}—

In some embodiments, Z₂ is —NH—.

In some embodiments, Z₂ is a covalent bond.

In some embodiments, Z₂ is —NH— and R^{2B} is Ring 2B.

As defined above and described herein, Z₃ is selected from a covalent bond, —O—, —NR^{C}—, —C(O)—, —CR^{C}₂—, —OC(O)—, —C(O)O—, —N(R^{C})C(O)—, and —C(O)N(R^{C})—.

In some embodiments, Z₃ is —NH—.

As defined above and described herein, Z₄ is selected from a covalent bond, —O—, —NR^{C}—, —C(O)—, —CR^{C}₂—, —OC(O)—, —C(O)O—, —N(R^{C})C(O)—, and —C(O)N(R^{C})—.

In some embodiments, Z₄ is a covalent bond, —O—, —C(O)—, —NH—, or —C(O)—N(CH₃)—.

As defined above and described herein, Z₅ is selected from a covalent bond, —O—, —NR^{C}—, —C(O)—, —CR^{C}₂—, —OC(O)—, —C(O)O—, —N(R^{C})C(O)—, and —C(O)N(R^{C})—.

In some embodiments, Z₅ is selected from a covalent bond and —O—.

As defined above and described herein, R^{C} is hydrogen or a C₁-C₆ aliphatic.

As defined above and described herein, R³ is a C₁-C₆ aliphatic hydrocarbon.

In some embodiments, R³ is an aliphatic C₁-C₄ hydrocarbon.

In some embodiments, R³ is —CH₃.

In some embodiments, -Z₃- is covalent bond and R³ is hydrogen.

In some embodiments, -Z₃- is —NR^{C}— and R³ is —CH₃.

As defined above and described herein, each R^{k} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, or: two R^{k} groups on the same nitrogen are optionally taken together with their intervening atoms to form a 4 to 7-membered saturated, partially unsaturated, or heteroaryl ring having 0-3 heteroatoms in addition to the nitrogen, independently selected from N, O, and S.

In some embodiments, R^{K} is independently hydrogen, —F, —Cl, —Br, —I, —OH, —O—(C₁-C₆ aliphatic), —NH₂, —NH—(C₁-C₆ aliphatic), —N(C₁-C₆ aliphatic)₂, —N(C₁-C₆ aliphatic)-OH, —N(C₁-C₆ aliphatic)-O—(C₁-C₆ aliphatic), —CN, —C(O)—C₁-C₆ aliphatic, —CHO, —CO₂H, —CO₂(C₁-C₆ aliphatic), —C(O)—NH₂, —C(O)—N(C₁-C₆ aliphatic)₂, —N(C₁-C₆ aliphatic)-C(O)—(C₁-C₆ aliphatic), —CF₃, —O—CF₃, a C₁-C₆ aliphatic group, or two R^{K} groups attached to the same carbon atom are optionally taken together to form ═O.

In some embodiments, R^{K} is —F, —Cl, —Br, —I, —OH, —NH₂, or a C₁-C₆ aliphatic group.

As defined above and described herein, Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S.

As defined above and described herein, Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S.

As defined above and described herein, Ring 2A is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-3 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof.

As defined above and described herein, Ring 2B is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S.

In some embodiments, TBM has a structure of Formula (IIBB′-1) or Formula (IIA′A′-1):

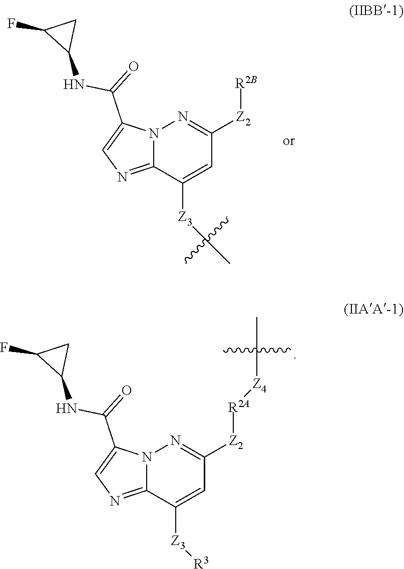

In some embodiments, TBM has a structure of Formula (IIA′-2-1):

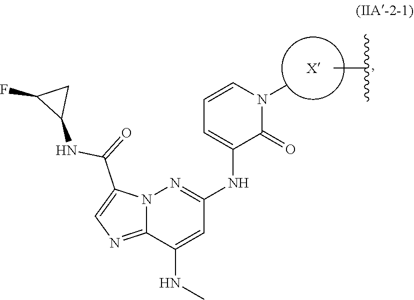

wherein Ring X′ is a ring selected from phenyl, a 4 to 7-membered saturated or partially unsaturated carbocyclyl or heterocyclyl with 1-3 heteroatoms independently selected from N, O, and S, and a 5 to 6-membered heteroaryl with 1-4 heteroatoms independently selected from N, O, and S, and each of which is optionally substituted with one or more of R^{K}.

In some embodiments, TBM has a structure of Formula (IIA′-2-a):

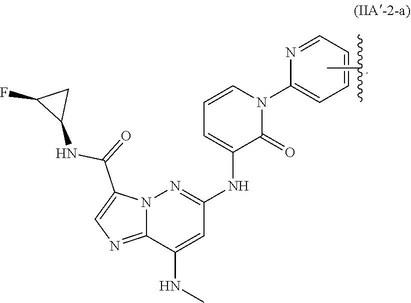

In some embodiments, TBM has a structure selected from:

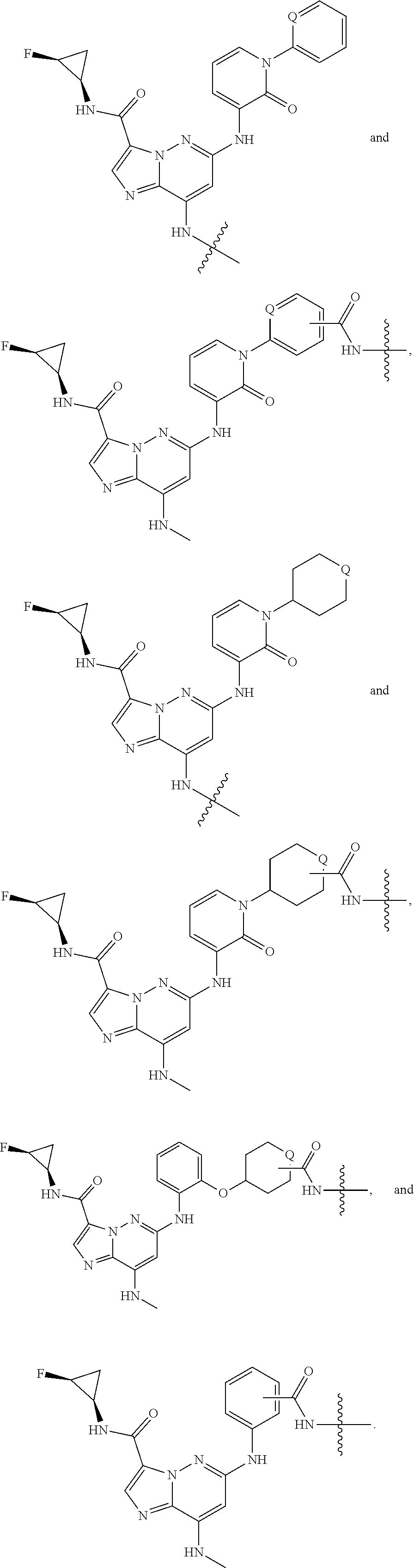

wherein Q is —C— or —N— and

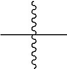

represents the point of attachment to L.

In some embodiments, TBM has a structure of Formula (IIA′-2-a):

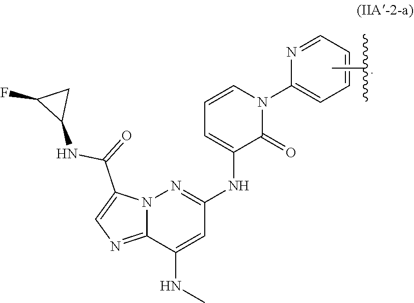

In some embodiments, TBM has a structure of Formula (IIA′-2-b):

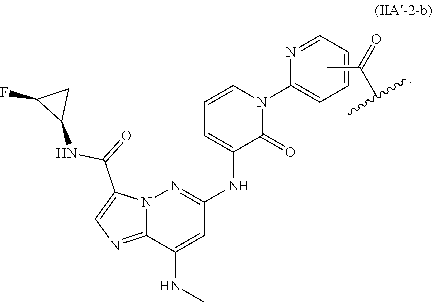

In some embodiments, TBM has a structure of Formula Formula (IIA′-2-c) or Formula (IIA′-2-d):

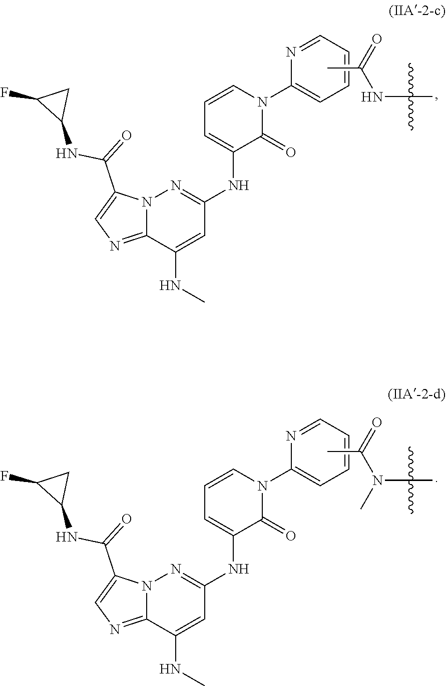

In some embodiments, TBM has a structure of Formula (IIA′-2-b′) or Formula (IIA′-2-c′):

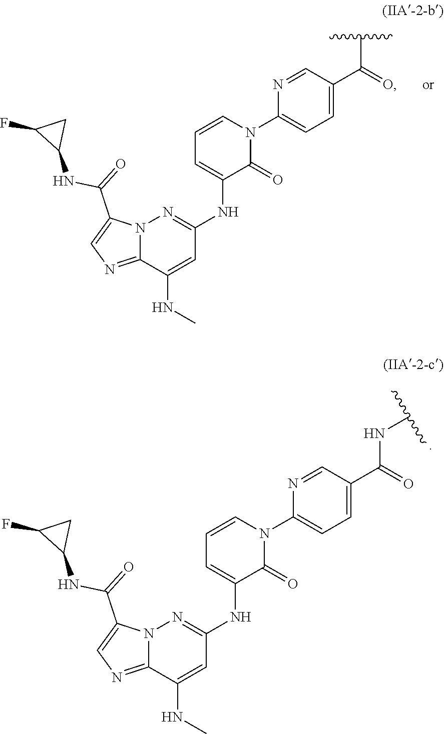 [SMILES: *C(=O)c1ccc(-n2cccc(Nc3cc(NC)c4ncc(C(=O)N[C@@H]5C[C@@H]5F)n4n3)c2=O)nc1 ; *NC(=O)c1ccc(-n2cccc(Nc3cc(NC)c4ncc(C(=O)N[C@@H]5C[C@@H]5F)n4n3)c2=O)nc1]

In some embodiments, TBM has a structure of Formula (IIIAA) or Formula (IIIBB):

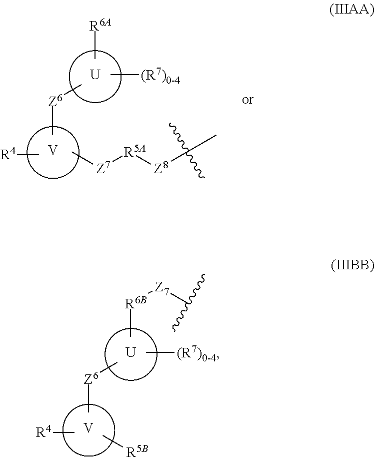

or a pharmaceutically acceptable salt thereof,
U is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
V is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
R⁴ is hydrogen, —F, —Cl, —Br, —I, —OH, —O—(C₁-C₆ aliphatic), —NH₂, —NH—(C₁-C₆ aliphatic), —N(C₁-C₆ aliphatic)₂, —CN, —C(O)—C₁-C₆ aliphatic, —CO₂H, —CO₂(C₁-C₆ aliphatic), —C(O)—NH₂, —C(O)—NH(C₁-C₆ aliphatic), —C(O)—N(C₁-C₆ aliphatic)₂, —C(O)—(C₁-C₆ aliphatic), —SO₂(C₁-C₆ aliphatic), —SO₂—N(C₁-C₆ aliphatic)₂, —S(O)—C₁-C₆ aliphatic, —CD₃, —CF₃, or —O—CF₃;
R^{5A} is selected from a covalent bond and Ring 5A, wherein the Ring 5A is optionally substituted with one or more R⁸;
R^{6A} is selected from a hydrogen, Ring 6A, and -Ring 6A′-Ring 6A, wherein the Ring 6A and the Ring 6A′ is independently optionally substituted with one or more R⁸;
R^{5B} is selected from a hydrogen, Ring 5B, and -Ring 5B′-Ring 5B, wherein the Ring 5B and the Ring 5B′ is independently optionally substituted with one or more R⁸;
R^{6B} is selected from a covalent bond and Ring 6B, wherein the Ring 6B is optionally substituted with one or more R⁸;
Ring 5A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
Ring 6A is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
Ring 6A′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S;
Ring 5B is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
Ring 5B′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S;
Ring 6B is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
Z⁶ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z⁷ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z⁸ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
each R⁷ is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—NH(C₁-C₆ aliphatic), —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CD₃, —CF₃, or —O—CF₃;
each R⁸ is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—NH(C₁-C₆ aliphatic), —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CD₃, —CF₃, or —O—CF₃;
each R^{C} is independently hydrogen or an optionally substituted C₁-C₆ aliphatic group,
and

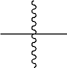

represents the point of attachment to L.

As defined above and described herein, U is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 5-6 membered heteroaryl having 1-4 heteroatoms independently selected from N, O, and S.

As defined above and described herein, V is an optionally substituted ring selected from a 5-6 membered heteroaryl having 1-4 heteroatoms independently selected from N, O, and S, and an 8-10 membered bicyclic heteroaryl having 1-5 heteroatoms independently selected from N, O, and S.

In some embodiments, TBM has a structure of Formula (IIIA′) or Formula (IIIB′):

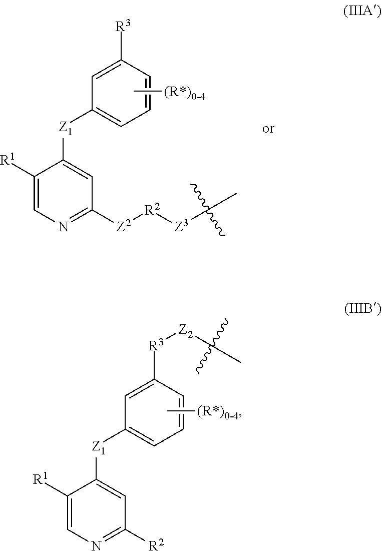

or a pharmaceutically acceptable salt thereof,
wherein R¹ is selected from a hydrogen, R*, an aliphatic C₁-C₂₀ hydrocarbon, -Cy-H, -Cy-C₁-C₂₀ aliphatic, and C₁-C₂₀ alkylene -Cy-H, wherein when R¹ is non hydrogen, R¹ further comprises 0-8 heteroatoms selected from halogen, O, N, and S, wherein R¹ is optionally substituted with one or more R*;
R² is selected from a covalent bond, C₁-C₂₀ alkylene, -Cy-, and —C₁-C₂₀ alkylene -Cy-, wherein when R² is C₁-C₂₀ alkylene, -Cy-, or —C₁-C₂₀ alkylene -Cy-, R² further comprises 0-8 heteroatoms selected from halogen, O, N, and S, and wherein R² is optionally substituted with one or more R*;
R³ is selected from a hydrogen, R*, an aliphatic C₁-C₂₀ hydrocarbon, -Cy-H, -Cy-C₁-C₂₀ aliphatic, and C₁-C₂₀ alkylene -Cy-H, wherein when R³ is non hydrogen, R³ further comprises 0-8 heteroatoms selected from halogen, O, N, and S, wherein R³ is optionally substituted with one or more R*;
Z₁, Z₂ and Z₃ are each independently selected from a covalent bond, —O—, —NR*—, —S—, —C(O)—, —C(S)—, —CR*₂—, —OC(O)—, —C(O)O—, —S(O)—, —S(O)₂—, —N(R*)S(O)₂—, —S(O)₂N(R*)—, —N(R*)C(O)—, —C(O)N(R*)—, —OC(O)N(R*)—, and —N(R*)C(O)O—;
-Cy- is an optionally substituted ring selected from phenylenyl, an 8-10 membered bicyclic arylenyl, a 3-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 4-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, and
each R* is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CD₃, —CF₃, —O—CF₃, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, or:
two R* groups on the same nitrogen are optionally taken together with their intervening atoms to form a 4 to 7-membered saturated, partially unsaturated, or heteroaryl ring having 0-3 heteroatoms in addition to the nitrogen, independently selected from N, O, and S,
and

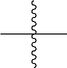

represents the point of attachment to L.

In some embodiments, R³ has a structure:

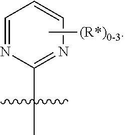

In some embodiments, TDM has the structure of Formula (IIIB), wherein R³ has a structure:

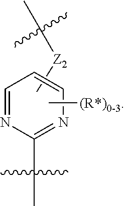

In some embodiments, TBM has a structure of Formula (IIIA) or Formula (IIIB):

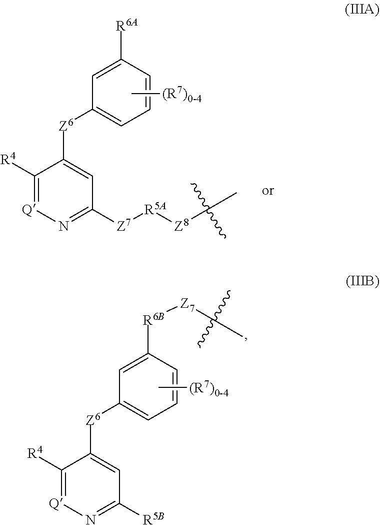

or a pharmaceutically acceptable salt thereof,
wherein Q′ is selected from —CH═ and —N═;
R⁴ is hydrogen, —F, —Cl, —Br, —I, —OH, —O—(C₁-C₆ aliphatic), —NH₂, —NH—(C₁-C₆ aliphatic), —N(C₁-C₆ aliphatic)₂, —CN, —C(O)—C₁-C₆ aliphatic, —CO₂H, —CO₂(C₁-C₆ aliphatic), —C(O)—NH₂, —C(O)—NH(C₁-C₆ aliphatic), —C(O)—N(C₁-C₆ aliphatic)₂, —C(O)—(C₁-C₆ aliphatic), —SO₂(C₁-C₆ aliphatic), —SO₂—N(C₁-C₆ aliphatic)₂, —S(O)—C₁-C₆ aliphatic, —CD₃, —CF₃, or —O—CF₃;
R^{5A} is selected from a covalent bond and Ring 5A, wherein the Ring 5A is optionally substituted with one or more R⁷;
R^{6A} is selected from a hydrogen, Ring 6A, and -Ring 6A′-Ring 6A, wherein the Ring 6A and the Ring 6A′ is independently optionally substituted with one or more R⁷;
R^{5B} is selected from a hydrogen, Ring 5B, and -Ring 5B′-Ring 5B, wherein the Ring 5B and the Ring 5B′ is independently optionally substituted with one or more R⁷;
R^{6B} is selected from a covalent bond and Ring 6B, wherein the Ring 6B is optionally substituted with one or more R⁷;
Ring 5A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
Ring 6A is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
Ring 6A′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S;
Ring 5B is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
Ring 5B′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S;
Ring 6B is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
Z⁶ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z⁷ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z⁸ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
each R⁷ is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—NH(C₁-C₆ aliphatic), —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CD₃, —CF₃, or —O—CF₃;
each R^{C} is independently hydrogen or an optionally substituted C₁-C₆ aliphatic group,
and

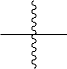

represents the point of attachment to L.

In some embodiments, TBM has a structure of Formula (IIIA-1) or Formula (IIIB-1):

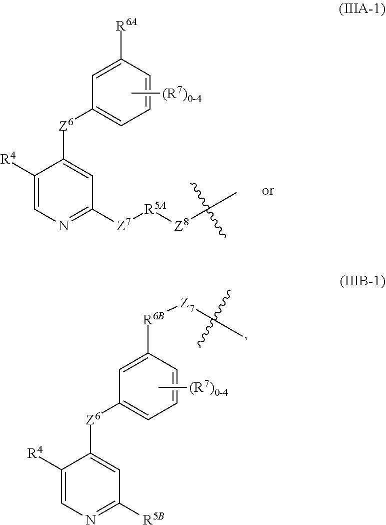

or a pharmaceutically acceptable salt thereof.

In some embodiments, TBM has a structure of Formula (IIIA-2) or Formula (IIIB-2):

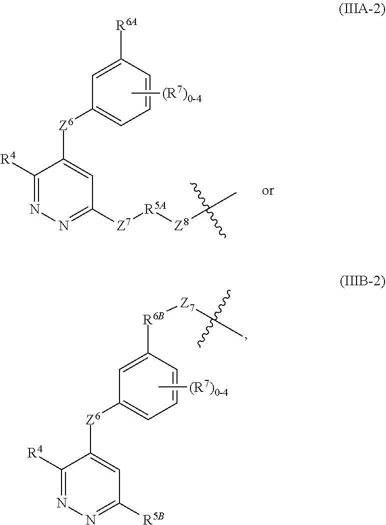

or a pharmaceutically acceptable salt thereof.

As defined above and described herein, R⁴ is hydrogen, —F, —Cl, —Br, —I, —OH, —O—(C₁-C₆ aliphatic), —NH₂, —NH—(C₁-C₆ aliphatic), —N(C₁-C₆ aliphatic)₂, —CN, —C(O)—C₁-C₆ aliphatic, —CO₂H, —C(O)—NH₂, —C(O)—NH(C₁-C₆ aliphatic), —C(O)—N(C₁-C₆ aliphatic)₂, —C(O)—(C₁-C₆ aliphatic), —CD₃, —CF₃, or —O—CF₃ in Formula (IIIA).

In some embodiments, R⁴ is hydrogen or —C(O)—NH(C₁-C₆ aliphatic) in Formula (IIIA).

In some embodiments, R⁴ is —C(O)—NH(CH₃) in Formula (IIIA).

In some embodiments, R⁴ is —C(O)—NH(CH₃).

As defined above and described herein, R^{6A} is Ring 6A.

As defined above and described herein, Ring 6A is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S.

As defined above and described herein, R^{6A} has a structure of

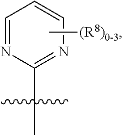

wherein each R⁸ is independently hydrogen, —F, —Cl, —Br, —I, —OH, or —O—(C₁-C₆ aliphatic) in Formula (IIIA).

In some embodiments, R^{6A} has a structure:

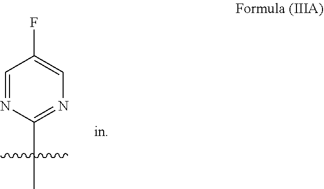 [SMILES: *c1ncc(F)cn1]

As defined above and described herein, R^{6B} is Ring 6B.

As defined above and described herein, Ring 6B is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S.

In some embodiments, R^{6B} has a structure:

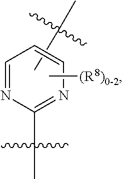

wherein each R⁸ is independently hydrogen, —F, —Cl, —Br, —I, —OH, or —O—(C₁-C₆ aliphatic) in Formula (IIIB).

As defined above and described herein, R⁸ is independently hydrogen, —F, —Cl, —Br, —I, —OH.

In some embodiments, at least one R⁸ is —F.

As defined above and described herein, each R⁷ is independently hydrogen, —F, —Cl, —Br, —I, —OH, or —O—(C₁-C₆ aliphatic).

In some embodiments, at least one R⁷ is non-hydrogen.

In some embodiments, one R⁷ is —O—(C₁-C₆ aliphatic).

In some embodiments, one R⁷ is —O—CH₃.

As defined above and described herein, Z⁶ is —NH—.

In some embodiments, the structure of Formula (IIIA-3) is:

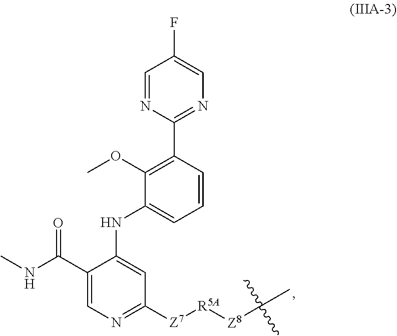

or a pharmaceutically acceptable salt thereof.

As defined above and described herein, Z⁷ is selected from a covalent bond, —NR^{C}—, —C(O)—, —NR^{C}C(O)—, —C(O)NR^{C}—, and —NR^{C}C(O)NR^{C}—.

As defined above and described herein, Z⁸ is selected from a covalent bond, —C(R^{C}₂)—, —NR^{C}—, —C(O)—, and —C(O)NR^{C}—.

In some embodiments, Z⁸ is —NH—.

In some embodiments, Z⁸ is selected from —C(O)NH— and —C(O)NCH₃—.

As defined above and described herein, R^{5A} is a covalent bond.

In some embodiments, R^{5A} is Ring 5A.

In some embodiments, R^{5A} is a 5-6 membered heteroarylenyl having 1-4 heteroatoms independently selected from N, O, and S.

In some embodiments, in Formula (IIIA), R^{5A} is

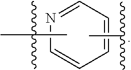

In some embodiments, TBM has a structure selected from:

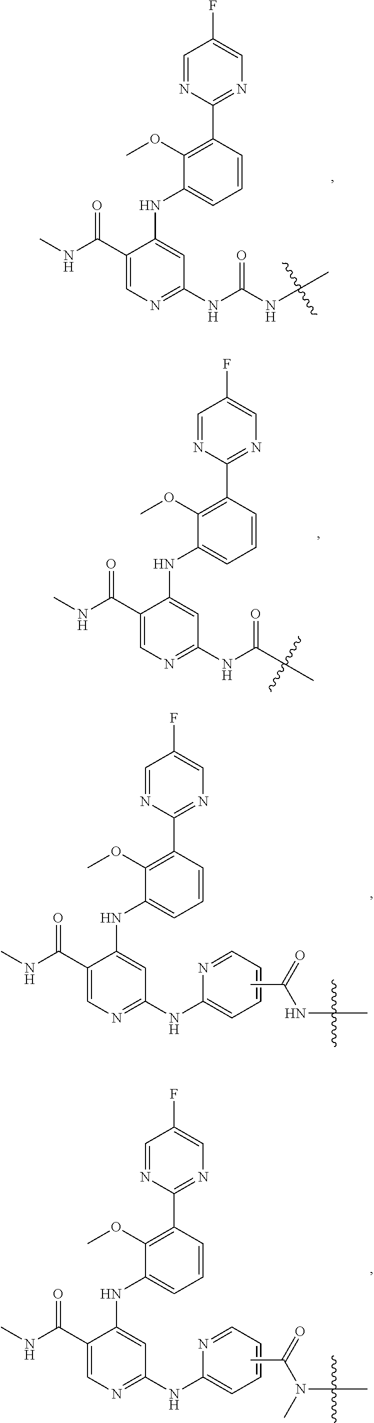

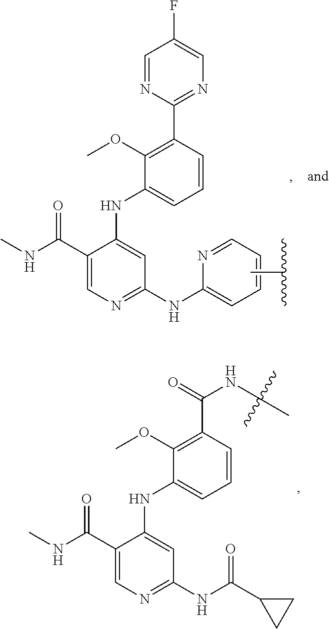

wherein

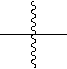

represents the point of attachment to L.

In some embodiments, TBM is:

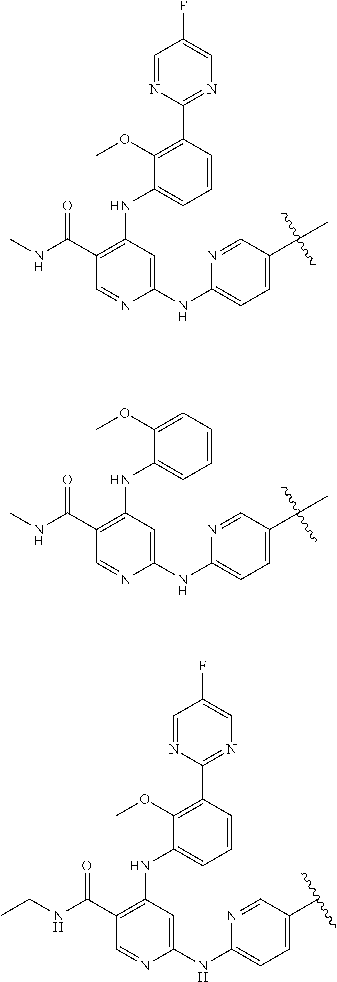 [SMILES: *c1ccc(Nc2cc(Nc3cccc(-c4ncc(F)cn4)c3OC)c(C(=O)NC)cn2)nc1 ; *c1ccc(Nc2cc(Nc3ccccc3OC)c(C(=O)NC)cn2)nc1 ; *c1ccc(Nc2cc(Nc3cccc(-c4ncc(F)cn4)c3OC)c(C(=O)NCC)cn2)nc1]

In some embodiments, TBM has a structure selected from:

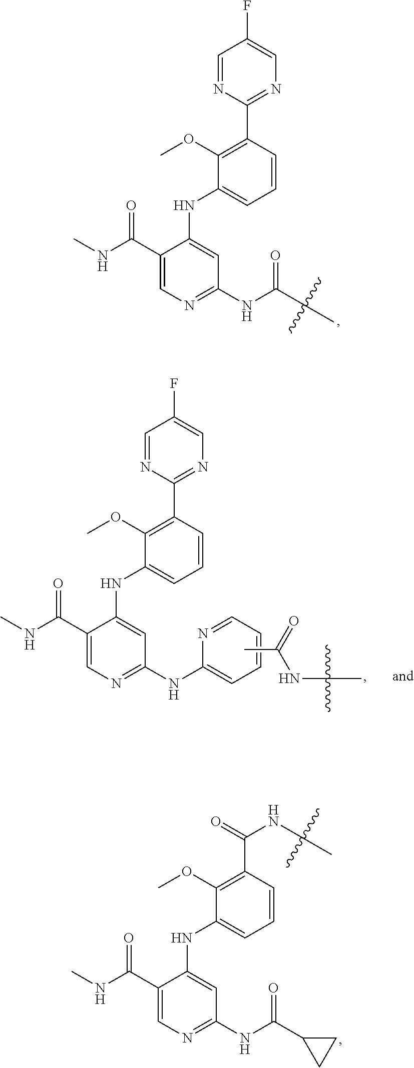

wherein

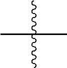

represents the point of attachment to L.

In some embodiments, a provided compound or pharmaceutically acceptable salt thereof, is selected from compounds 1.1-1.8, shown below:

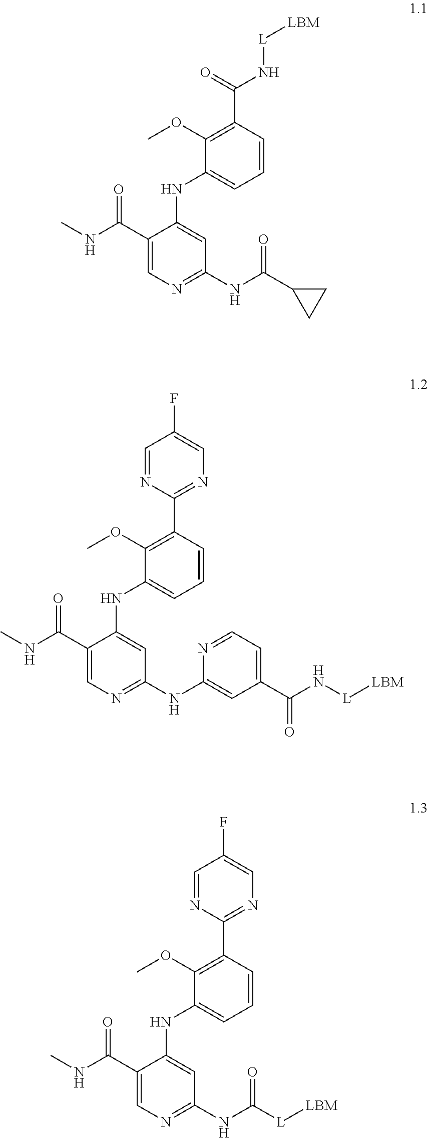

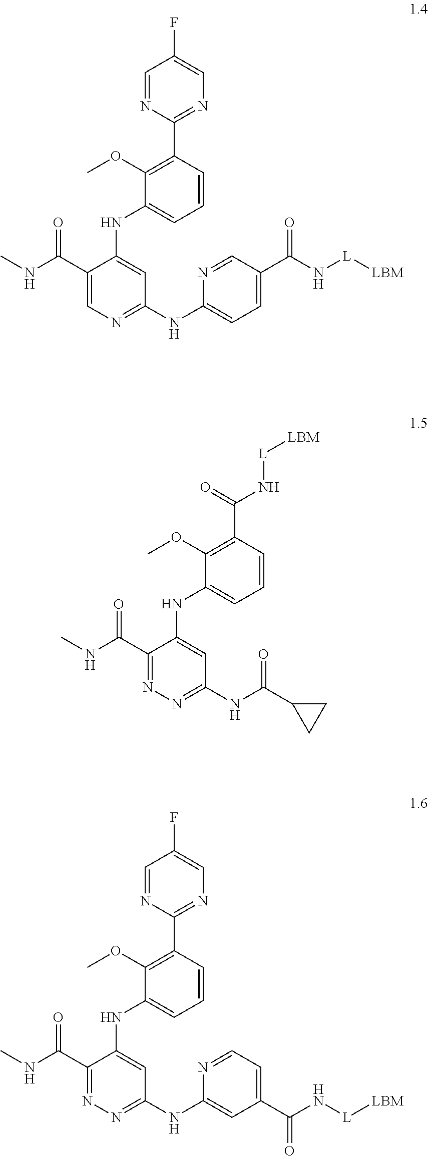

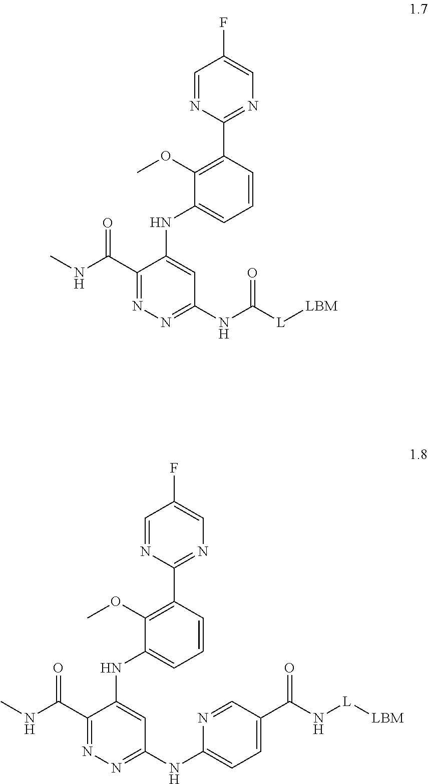

wherein LBM is selected from any of those in Table A below, and L is selected from any of those in Table B below.

In some embodiments, a provided compound, or pharmaceutically acceptable salt thereof, is selected from compounds 2.1-2.7, shown below:

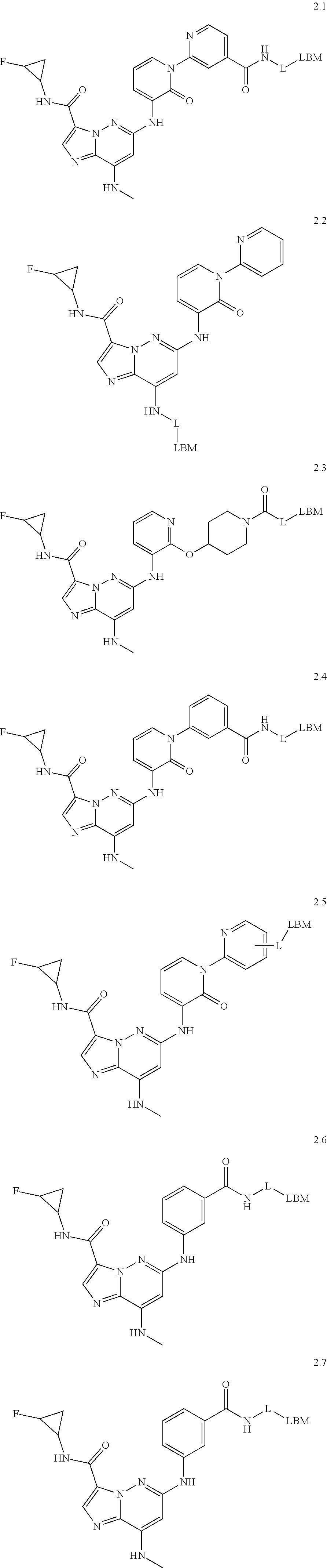

wherein LBM is selected from any of those in Table A below, and L is selected from any of those in Table B below.

In some embodiments, a provided compound, or pharmaceutically acceptable salt thereof, is selected from compounds 2.1A-2.7A, shown below:

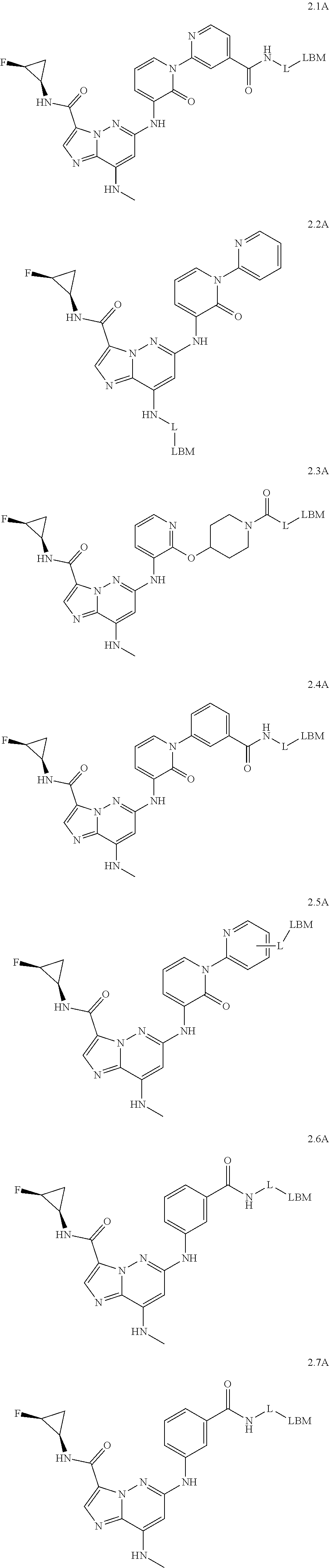

wherein LBM is selected from any of those in Table A below, and L is selected from any of those in Table B below.

In some embodiments, TBM is selected from the group consisting of BMS-986165, BMS-986202, PF-06826647.

In some embodiments, the present invention provides a compound of Formula I, wherein TBM is a binding moiety as recited in WO 2022/136914, thereby forming a compound of Formula (I-x-1):

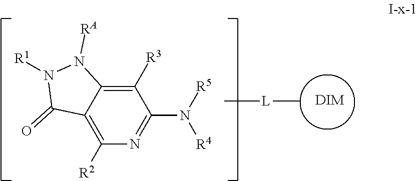

or a pharmaceutically acceptable salt thereof, wherein L and DIM are as defined below and described in embodiments herein, wherein each of the variables R¹, R², R³, R⁴, R⁵, and R^{A} is as described and defined in WO 2022/136914, the entirety of each of which is herein incorporated by reference.

In some embodiments, the present invention provides a compound of Formula I, wherein TBM is a binding moiety as recited in WO 2022/109580, thereby forming a compound of Formula (I-x-2):

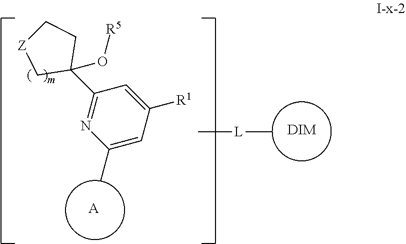

or a pharmaceutically acceptable salt thereof, wherein L and DIM are as defined below and described in embodiments herein, wherein each of the variables A, R¹, R⁵, Z, and m is as described and defined in WO 2022/109580, the entirety of each of which is herein incorporated by reference.

In some embodiments, the present invention provides a compound of Formula I, wherein TBM is a binding moiety as recited in WO 2022/083560, thereby forming a compound of Formula (I-x-3):

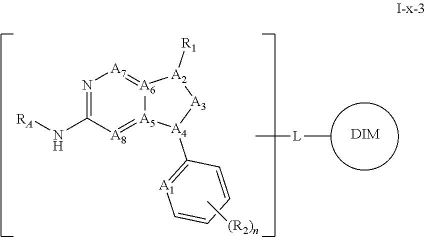

or a pharmaceutically acceptable salt thereof, wherein L and DIM are as defined below and described in embodiments herein, wherein each of the variables R₁, R₂, R₄, A₁, A₂, A₃, A₄, A₅, A₆, A₇, A₈, and n is as described and defined in WO 2022/083560, the entirety of each of which is herein incorporated by reference.

In some embodiments, the present invention provides a compound of Formula I, wherein TBM is a binding moiety as recited in CN 114075220, thereby forming a compound of Formula (I-x-4):

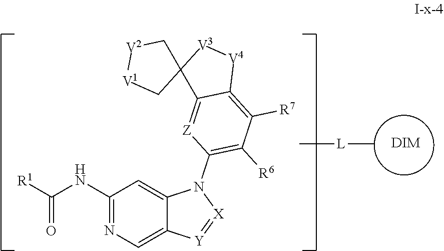

or a pharmaceutically acceptable salt thereof, wherein L and DIM are as defined below and described in embodiments herein, wherein each of the variables R¹, R⁶, R⁷, V¹, V², V³, V⁴, X, Y, and Z is as described and defined in CN 114075220, the entirety of each of which is herein incorporated by reference.

In some embodiments, the present invention provides a compound of Formula I, wherein TBM is a binding moiety as recited in CN 114075194, thereby forming a compound of Formula (I-x-5):

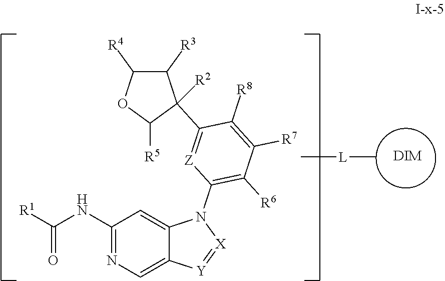

or a pharmaceutically acceptable salt thereof, wherein L and DIM are as defined below and described in embodiments herein, wherein each of the variables R¹, R², R³, R⁴, R⁵, R⁶, R⁷, R⁸, X, Y, and Z is as described and defined in CN 114075194, the entirety of each of which is herein incorporated by reference.

In some embodiments, the present invention provides a compound of Formula I, wherein TBM is a binding moiety as recited in WO 2019/178079, thereby forming a compound of Formula (I-x-6):

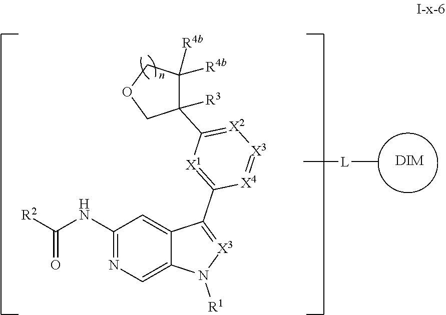

or a pharmaceutically acceptable salt thereof, wherein L and DIM are as defined below and described in embodiments herein, wherein each of the variables R¹, R², R³, R^{4a}, R^{4b}, X¹, X², X³, X⁴, and X⁵ is as described and defined in WO 2019/178079, the entirety of each of which is herein incorporated by reference.

### Degradation Inducing Moiety (DIM)

In one aspect, the present invention provides a compound of Formula (I):

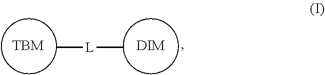

or a pharmaceutically acceptable salt thereof, wherein TBM is a TYK binding moiety capable of binding to TYK2 protein; L is a bivalent moiety that connects TBM to DIM; and DIM is a degradation-inducing moiety selected from a ligase binding moiety (LBM) and a lysine mimetic, or a hydrogen atom.

In some embodiments, DIM is LBM as described below and herein. In some embodiments, DIM is a lysine mimetic. In some embodiments, the covalent attachment of ubiquitin to TYK2 protein is achieved through the action of a lysine mimetic. In some embodiments, upon the binding of a compound of Formula (I) to TYK2 protein, the moiety that mimics a lysine undergoes ubiquitination thereby marking TYK2 protein for degradation via the Ubiquitin-Proteasome Pathway (UPP

In some embodiments, DIM is

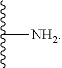 [SMILES: *N]

In some embodiments, DIM is

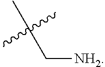 [SMILES: *CN]

In some embodiments, DIM is

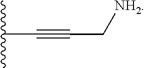 [SMILES: *C#CCN]

In some embodiments, DIM is selected from LBM depicted in Table A, below.

In some embodiments, the present invention provides the compound of Formula (I) as a compound of Formula (I-aaaa-NH₂):

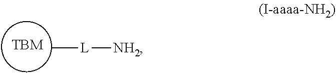

or a pharmaceutically acceptable salt thereof, wherein each of TBM and L is as defined above and described in embodiments herein, both singly and in combination.

In some embodiments, the present invention provides the compound of Formula (I) as a compound of Formula (I-aaaa-CH₂—NH₂):

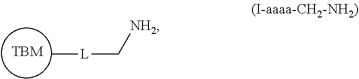

or a pharmaceutically acceptable salt thereof, wherein each of TBM and L is as defined above and described in embodiments herein, both singly and in combination.

In some embodiments, the present invention provides the compound of Formula (I) as a compound of Formula (I-aaaa-CC—CH₂—NH₂):

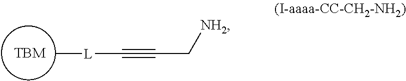

or a pharmaceutically acceptable salt thereof, wherein each of TBM and L is as defined above and described in embodiments herein, both singly and in combination.
In certain embodiments, the present invention provides a compound of Formula (I), wherein DIM is a lysine mimetic. In certain embodiments, the lysine mimetic is a compound of Formulae I-bbbb-1, I-bbbb-2, or I-bbbb-3:

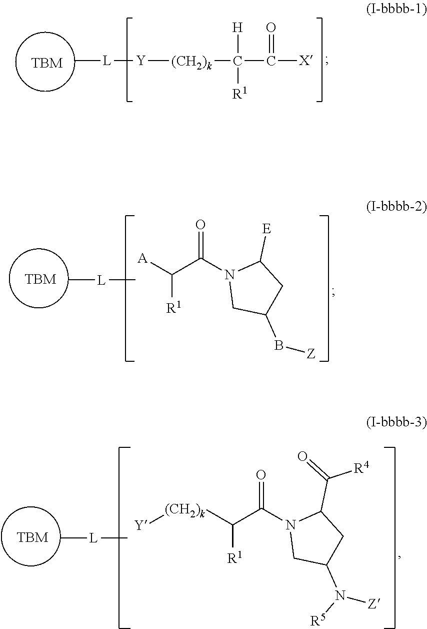

or a pharmaceutically acceptable salt thereof, wherein L and TBM are as defined above and described in embodiments herein, and wherein each of the variables R¹, R⁴, R⁵, A, B, E, Y, Y′, Z, Z′, and k are as defined and described in U.S. Pat. No. 7,622,496, the entirety of each of which is herein incorporated by reference.

In some embodiments, DIM is a hydrogen atom. In some embodiments, the covalent attachment of ubiquitin to TYK2 protein is achieved through a provided compound wherein DIM is a hydrogen atom. In some embodiments, upon the binding of a compound of Formula (I) to TYK2 protein, the moiety being hydrogen effectuates ubiquitination thereby marking TYK2 protein for degradation via the Ubiquitin-Proteasome Pathway (UPP).

In some embodiments, the present invention provides the compound of Formula (I) wherein DIM is a hydrogen atom, thereby forming a compound of Formula (I-cccc):

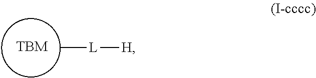

or a pharmaceutically acceptable salt thereof, wherein each of TBM and L is as defined above and described in embodiments herein, both singly and in combination.

### Ligase-Binding Moiety (LBM)

In some embodiments, LBM is an E3 ligase ligand. Such E3 ligase ligands are well known to one of ordinary skill in the art and include those described in M. Toure, C. M. Crews, Angew. Chem. Int. Ed. 2016, 55, 1966, T. Uehara et al. Nature Chemical Biology 2017, 13, 675, WO 2017/176708, US 2017/0281784, WO 2017/161119, WO 2017/176957, WO 2017/176958, WO 2015/160845, US 2015/0291562, WO 2016/197032, WO 2016/105518, US 2018/0009779, WO 2017/007612, 2018/0134684, WO 2013/106643, US 2014/0356322, WO 2002/020740, US 2002/0068063, WO 2012/078559, US 2014/0302523, WO 2012/003281, US 2013/0190340, US 2016/0022642, WO 2014/063061, US 2015/0274738, WO 2016/118666, US 2016/0214972, WO 2016/149668, US 2016/0272639, WO 2016/169989, US 2018/0118733, WO 2016/197114, US 2018/0147202, WO 2017/011371, US 2017/0008904, WO 2017/011590, US 2017/0037004, WO 2017/079267, US 2017/0121321, WO 2017/117473, WO 2017/117474, WO 2013/106646, WO 2014/108452, WO 2017/197036, US 2019/0076540, WO 2017/197046, US 2019/0076542, WO 2017/197051, US 2019/0076539, WO 2017/197055, US 2019/0076541, and WO 2017/197056, the entirety of each of which is herein incorporated by reference.

As defined herein and described below, wherein a formula is depicted using square brackets, e.g,

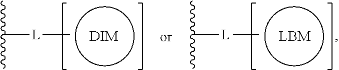

L is attached to a modifiable carbon, oxygen, or nitrogen atom within DIM or LBM including substitution or replacement of a defined group in DIM or LBM.

In some embodiments, LBM is an E3 ubiquitin ligase binding moiety.

In some embodiments, the E3 ubiquitin ligase binding moiety is a cereblon E3 ubiquitin ligase binding moiety, a VHL E3 ubiquitin ligase binding moiety, an IAP E3 ubiquitin ligase binding moiety, or an MDM2 E3 ubiquitin ligase binding moiety.

In certain embodiments, the present invention provides a compound of Formula (I), wherein LBM is an IMiD-based (immunomodulatory imide drug-based) cereblon E3 ubiquitin ligase binding moiety thereby forming a compound of Formula I-a-1, I-a-2, I-a-3, I-a-4, I-a-5, I-a-6, I-a-7, I-a-8, I-a-9, I-a-10, or I-a-11 respectively:

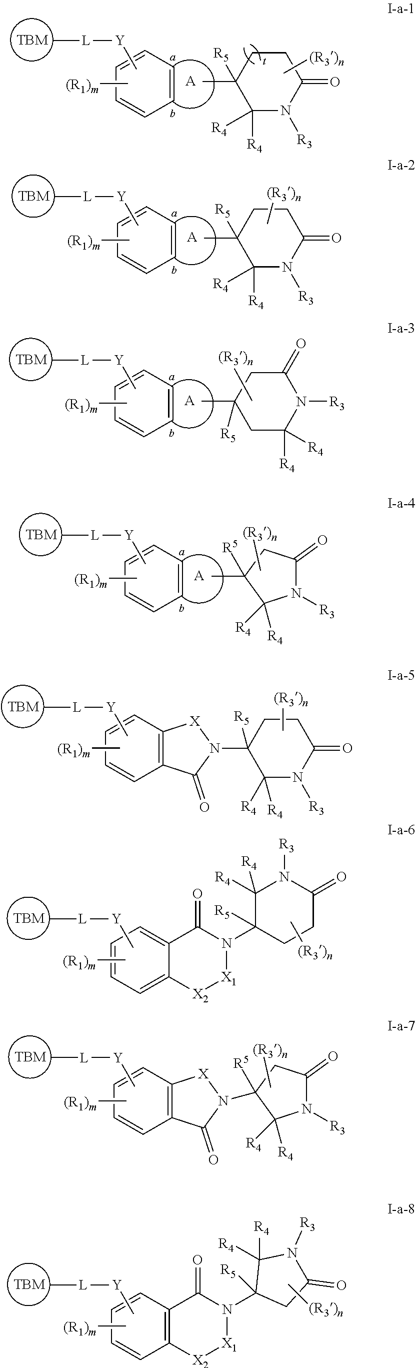

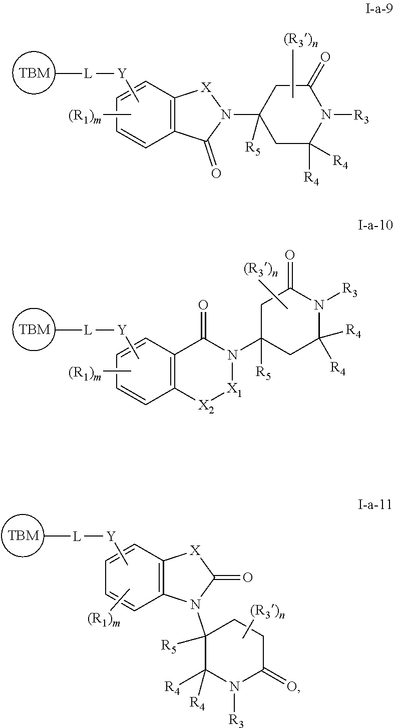

or a compound of Formula I-a′-1, I-a′-2, I-a′-3, I-a′-4, I-a′-5, I-a′-6, I-a′-7, I-a′-8, I-a′-9, I-a′-10, or I-a′-11, respectively:

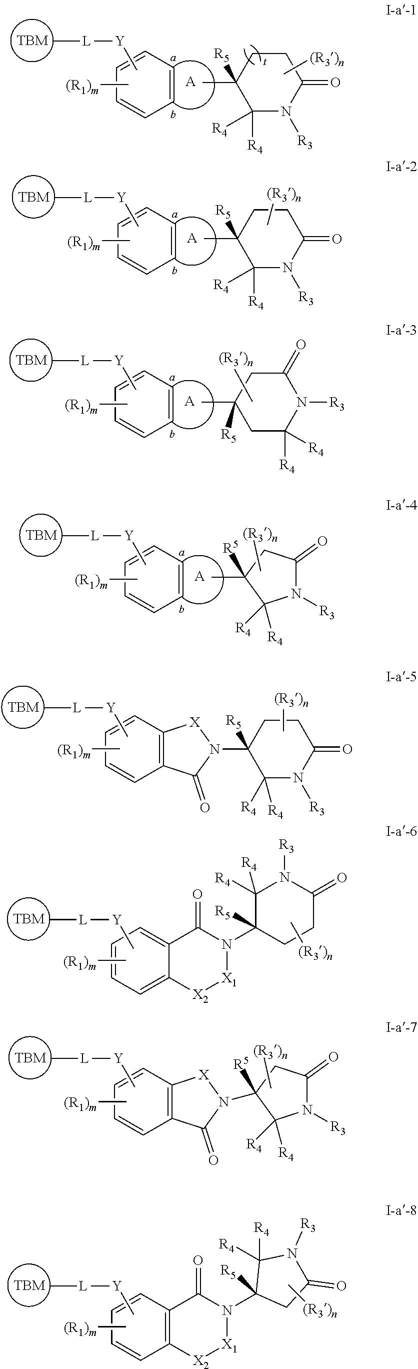

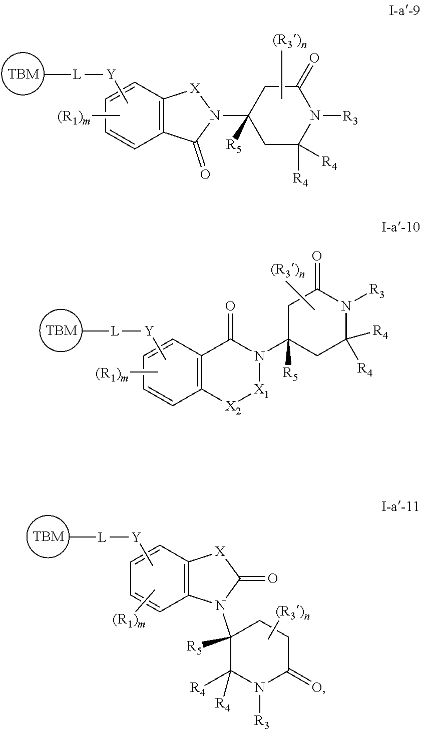

or a compound of Formula I-a″-1, I-a″-2, I-a″-3, I-a″-4, I-a″-5, I-a″-6, I-a″-7, I-a″-8, I-a″-9, or I-a″-10 respectively:

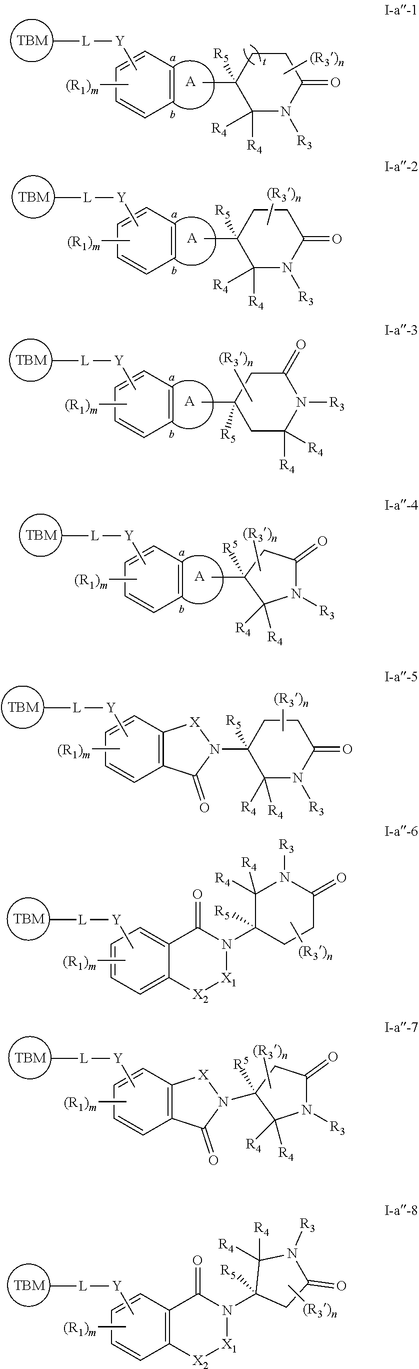

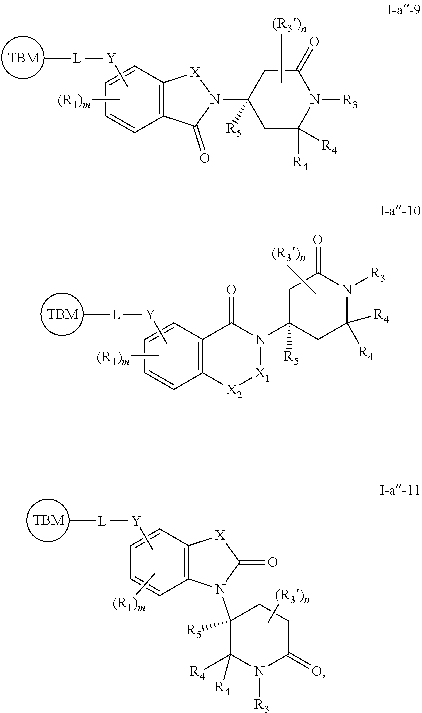

or a pharmaceutically acceptable salt thereof, wherein L and TBM are as defined above and described in embodiments herein, and wherein:

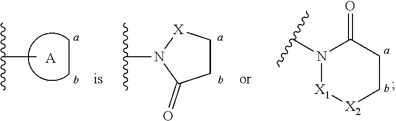

Y is a bond, Y₁, O, NH, NR₂, C(O)O, OC(O), C(O)NR₂′, NR₂′C(O), Y₁—O, Y₁—NH, Y₁—NR₂, Y₁—C(O), Y₁—C(O)O, Y₁—OC(O), Y₁—C(O)NR₂′, or Y₁—NR₂′C(O), wherein Y₁ is C₁-C₆ alkylene, C₂-C₆ alkenylene, or C₂-C₆ alkynylene;
X is C(O) or C(R³)₂;
X₁-X₂ is C(R₃)═N or C(R³)₂—C(R³)₂;
each R₁ is independently halogen, nitro, NH₂, OH, C(O)OH, C₁-C₆ alkyl, or C₁-C₆ alkoxy;
R₂ is C₁-C₆ alkyl, C₂-C₆ alkenyl, C₃-C₈ cycloalkyl, 3- to 8-membered heterocycloalkyl, C(O)—C₁-C₆ alkyl, C(O)—C₂-C₆ alkenyl, C(O)—C₃-C₈ cycloalkyl, or C(O)-3- to 8-membered heterocycloalkyl, and R₂ is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy;
R₂′ is H, C₁-C₆ alkyl, C₂-C₆ alkenyl, C₃-C₈ cycloalkyl, or 3- to 8-membered heterocycloalkyl, and R₂′, when not being H, is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy;
each R₃ is independently H or C₁-C₃ alkyl optionally substituted with C₆-C₁₀ aryl or 5- to 10-membered heteroaryl;
each R₃′ is independently C₁-C₃ alkyl;
each R₄ is independently H or C₁-C₃ alkyl; or two R₄, together with the carbon atom to which they are attached, form C(O), a C₃-C₆ carbocycle, or a 4-, 5-, or 6-membered heterocycle comprising 1 or 2 heteroatoms selected from N and O;
R₅ is H, C₁-C₃ alkyl, F, or Cl;
each Rₐ independently is H or C₁-C₆ alkyl;
R_{b} is H or tosyl;
t is 0 or 1;
m is 0, 1, 2 or 3; and
n is 0, 1 or 2.

In some embodiments, LBM is

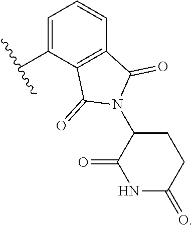 [SMILES: *c1cccc2c1C(=O)N(C1CCC(=O)NC1=O)C2=O]

In some embodiments, LBM is

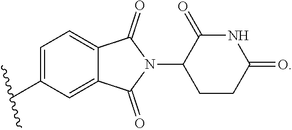 [SMILES: *c1ccc2c(c1)C(=O)N(C1CCC(=O)NC1=O)C2=O]

In some embodiments, LBM is

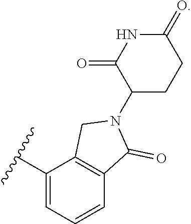 [SMILES: *c1cccc2c1CN(C1CCC(=O)NC1=O)C2=O]

In some embodiments, LBM is

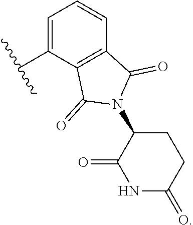 [SMILES: *c1cccc2c1C(=O)N([C@H]1CCC(=O)NC1=O)C2=O]

In some embodiments, LBM is

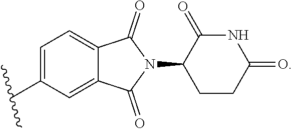 [SMILES: *c1ccc2c(c1)C(=O)N([C@@H]1CCC(=O)NC1=O)C2=O]

In some embodiments, LBM is

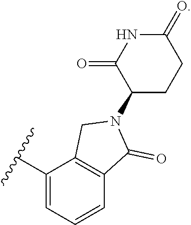 [SMILES: *c1cccc2c1CN([C@@H]1CCC(=O)NC1=O)C2=O]

In some embodiments, LBM is

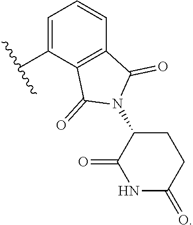 [SMILES: *c1cccc2c1C(=O)N([C@@H]1CCC(=O)NC1=O)C2=O]

In some embodiments, LBM is

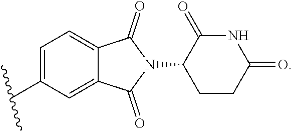 [SMILES: *c1ccc2c(c1)C(=O)N([C@H]1CCC(=O)NC1=O)C2=O]

In some embodiments, LBM is

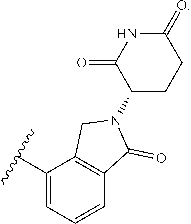 [SMILES: *c1cccc2c1CN([C@H]1CCC(=O)NC1=O)C2=O]

In certain embodiments, the present invention provides a compound of Formula I, wherein LBM is a cereblon E3 ubiquitin ligase binding moiety thereby forming a compound of Formula I-b:

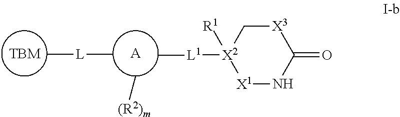

or a pharmaceutically acceptable salt thereof, wherein L and TBM are as defined above and described herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

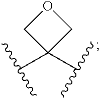 [SMILES: *C1(*)COC1]

X² is a carbon atom or silicon atom;
X³ is a bivalent moiety selected from —CR₂—, —NR—, —O—, —S—, or —Si(R)₂—;
R¹ is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring A is a bi- or tricyclic ring selected from

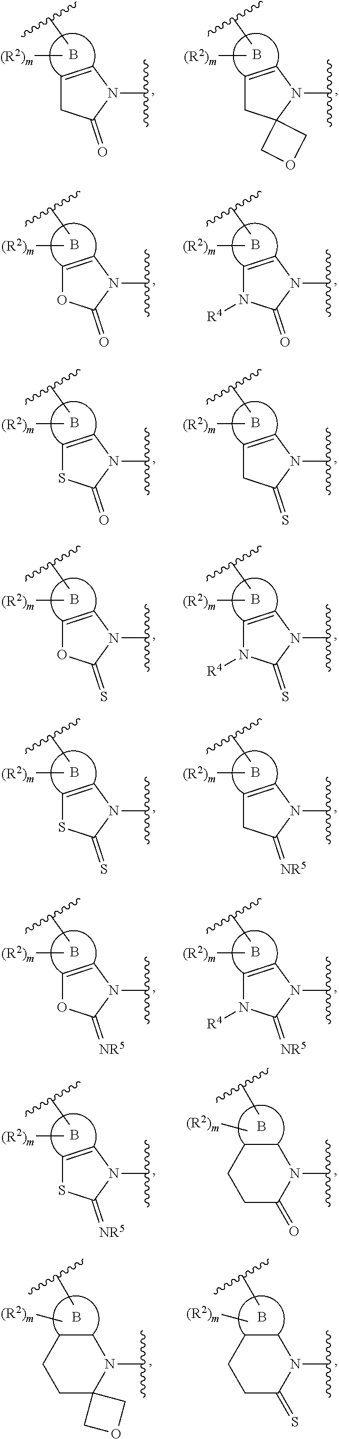

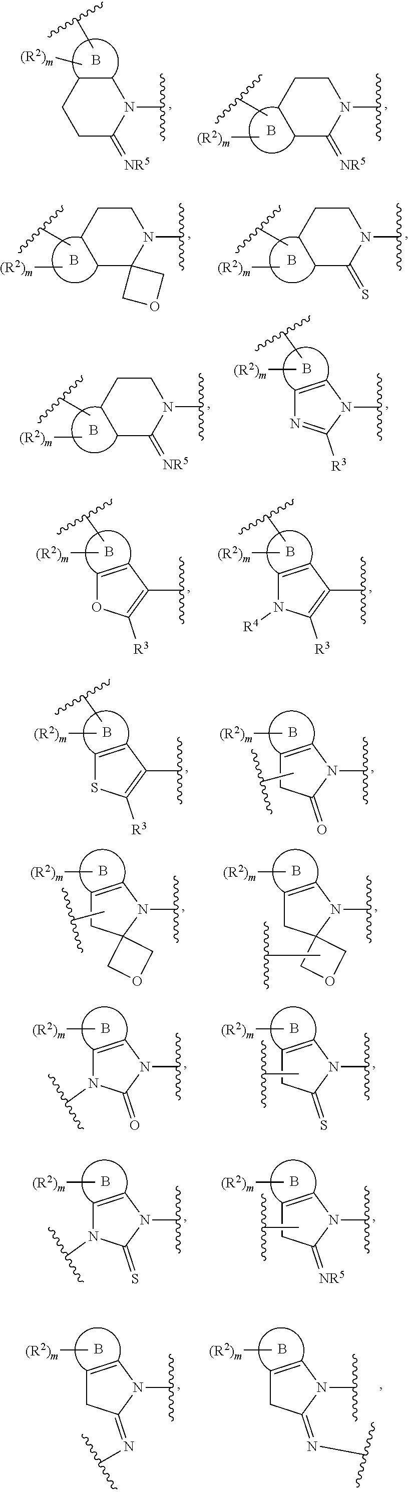

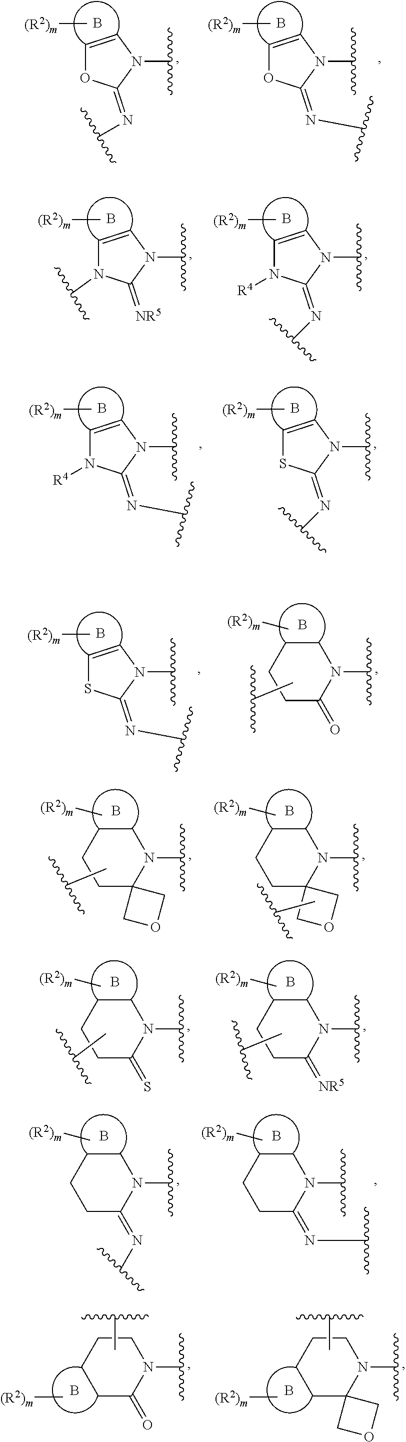

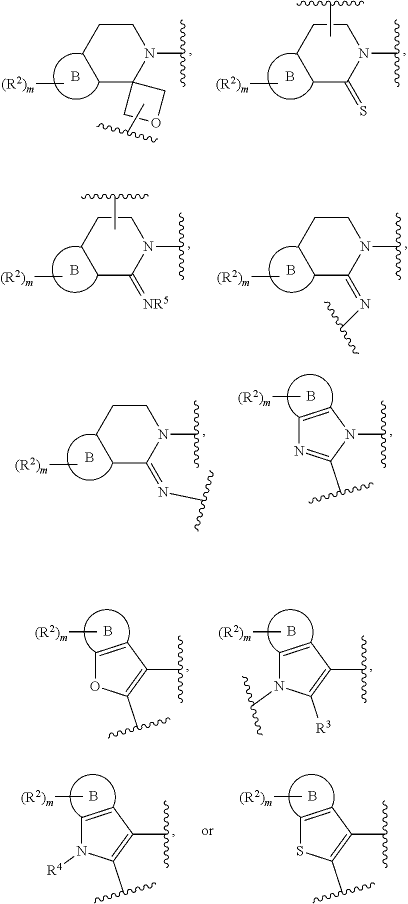

wherein Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
R³ is selected from hydrogen, halogen, —OR, —N(R)₂, or —SR;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or 4;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

Where a point of attachment of —(R²)ₘ is depicted on Ring B, it is intended, and one of ordinary skill in the art would appreciate, that the point of attachment of —(R²)ₘ may be on Ring A and may also be at any available carbon or nitrogen atom on Ring A including the ring to which Ring B is fused. Where —R² is attached to a nitrogen atom bound to R⁴ or R⁵, R⁴ or R⁵ is absent and —R² takes the place of the R⁴ or R⁵ group. Where —R² is attached to a carbon atom bound to R³, R³ is absent and —R² takes the place of the R³ group.

In some embodiments, a compound of Formula I-b above is provided as a compound of Formula I-b-1 or Formula I-b-2:

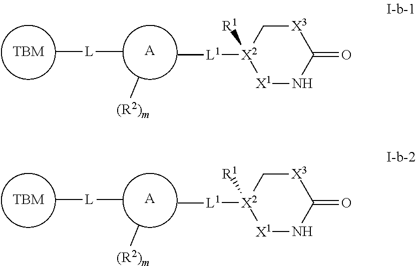

or a pharmaceutically acceptable salt thereof, wherein:
each of TBM, Ring A, L, L¹, R¹, R², X¹, X², X³, and m is as defined above.

In some embodiments, a compound of Formula I-b above is provided as a compound of Formula I-b-3:

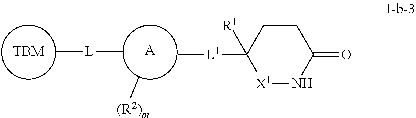

or a pharmaceutically acceptable salt thereof, wherein:
each of TBM, Ring A, L, R¹, R², X¹, and m is as defined above.

In some embodiments, a compound of Formula I-b above is provided as a compound of Formula I-b-4:

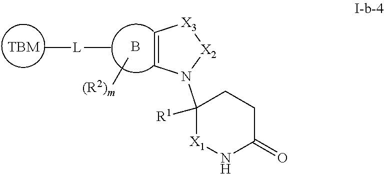

or a pharmaceutically acceptable salt thereof, wherein:
each of X₁, X₂, and X₃ is independently a covalent bond, —CH₂—, —C(O)—, —C(S)—, —NR— or

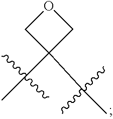 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic group;
each of R² is independently at each occurrence hydrogen, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or
—N(R)S(O)₂R;
Ring B is a fused ring selected from a 6-membered aryl containing 0-2 nitrogen atoms, a 5 to 7-membered partially saturated carbocyclyl, a 5 to 7-membered partially saturated heterocyclyl having 1-2 heteroatoms independently selected from nitrogen, oxygen or sulfur, or a 5-membered heteroaryl having 1-3 heteroatoms independently selected from nitrogen, oxygen or sulfur;
m is an integer from 0 to 4;
each R is independently at each occurrence hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, and combinations thereof, or:
two R groups on the same nitrogen are optionally taken together with their intervening atoms to form a 4 to 7-membered saturated, partially unsaturated, or heteroaryl ring having 0-3 heteroatoms in addition to the nitrogen, independently selected from N, O, and S.

In some embodiments, X₁ and X₂ are —C(O)— and X₃ is —NR—, wherein R is hydrogen, or an optionally substituted C₁-C₆ aliphatic group.

In some embodiments, R¹ and R² are a hydrogen at each occurrence.

In some embodiments, Ring A is a fused phenyl ring.

In some embodiments, Formula (I) has a structure selected from Formulas I-a-11, I-a′-11, and I-a″-11:

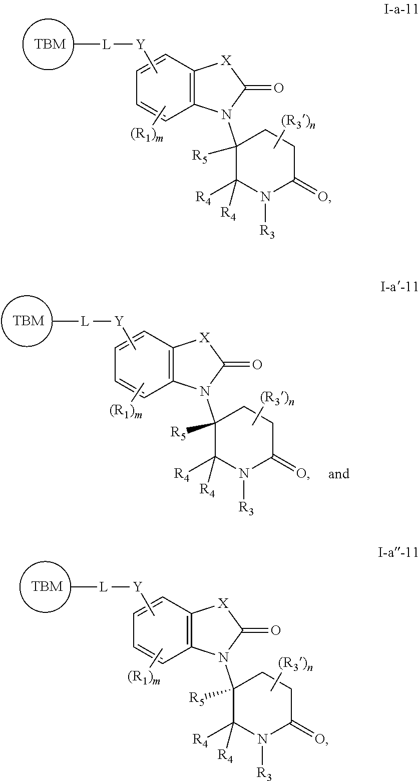

or a pharmaceutically acceptable salt thereof,
wherein L and TBM are as defined above, and wherein:
Y is a bond, Y₁, O, NH, NR₂, C(O)O, OC(O), C(O)NR₂′, NR₂′C(O), Y₁—O, Y₁—NH, Y₁—NR₂, Y₁—C(O), Y₁—C(O)O, Y₁—OC(O), Y₁—C(O)NR₂′, or Y₁—NR₂′C(O), wherein Y₁ is C₁-C₆ alkylene, C₂-C₆ alkenylene, or C₂-C₆ alkynylene;
X is C(O) or C(R³)₂;
each R₁ is independently halogen, nitro, NH₂, OH, C(O)OH, C₁-C₆ alkyl, or C₁-C₆ alkoxy;
R₂ is C₁-C₆ alkyl, C₂-C₆ alkenyl, C₃-C₈ cycloalkyl, 3- to 8-membered heterocycloalkyl, C(O)—C₁-C₆ alkyl, C(O)—C₂-C₆ alkenyl, C(O)—C₃-C₈ cycloalkyl, or C(O)-3- to 8-membered heterocycloalkyl, and R₂ is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy;
R₂′ is H, C₁-C₆ alkyl, C₂-C₆ alkenyl, C₃-C₈ cycloalkyl, or 3- to 8-membered heterocycloalkyl, and R₂′, when not being H, is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy;
each R₃ is independently H or C₁-C₃ alkyl optionally substituted with C₆-C₁₀ aryl or 5- to 10-membered heteroaryl;
each R₃′ is independently C₁-C₃ alkyl;
each R₄ is independently H or C₁-C₃ alkyl; or two R₄, together with the carbon atom to which they are attached, form C(O), a C₃-C₆ carbocycle, or a 4-, 5-, or 6-membered heterocycle comprising 1 or 2 heteroatoms selected from N and O;
R₅ is H, C₁-C₃ alkyl, F, or Cl;
each Rₐ independently is H or C₁-C₆ alkyl;
R_{b} is H or tosyl;
t is 0 or 1;
m is 0, 1, 2 or 3; and
n is 0, 1 or 2.

In some embodiments, the E3 ubiquitin ligase binding moiety is selected from the group consisting of:

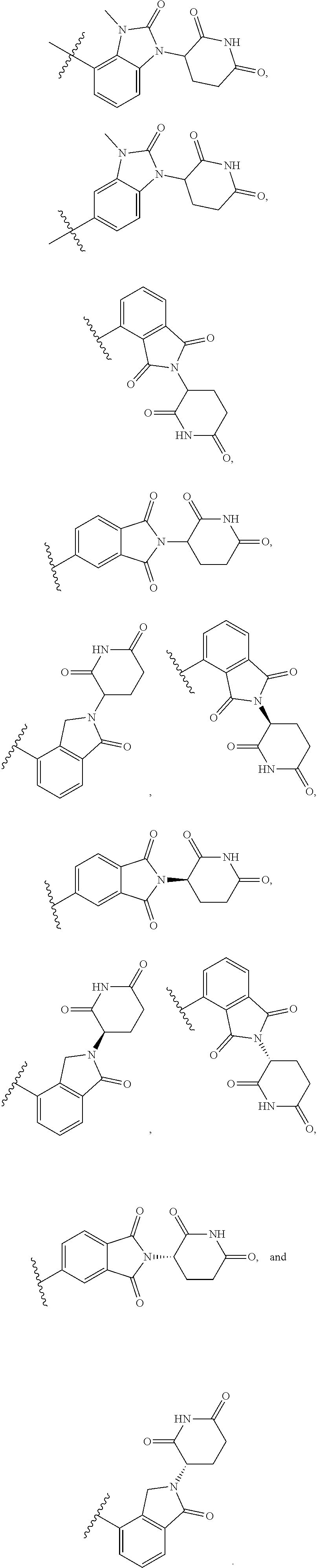

In some embodiments, the E3 ubiquitin ligase binding moiety is

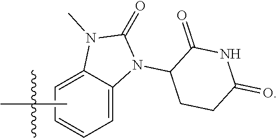

In some embodiments, the E3 ubiquitin ligase binding moiety is

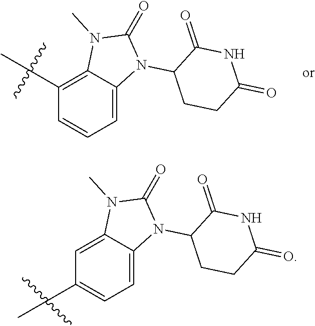

In some embodiments, Formula (I) has a structure of:

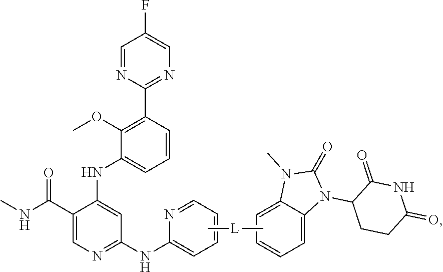

or a pharmaceutically acceptable salt thereof.

In some embodiments, Formula (I) has a structure of:

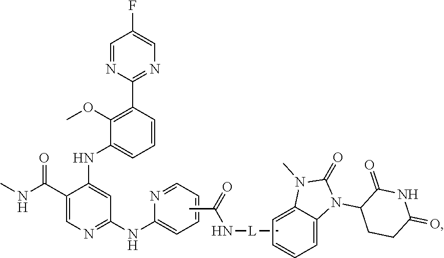

or a pharmaceutically acceptable salt thereof.

In some embodiments, Formula (I) has a structure of:

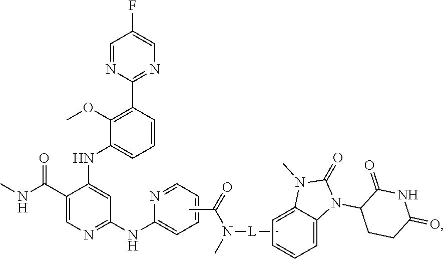

or a pharmaceutically acceptable salt thereof.

In some embodiments, Formula (I) has a structure selected from the group consisting of:

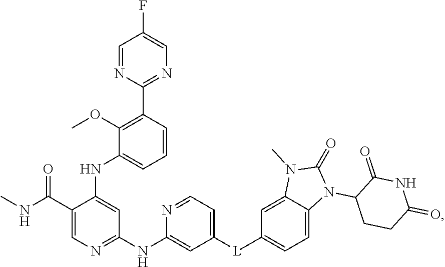

or a pharmaceutically acceptable salt thereof.

In some embodiments, Formula (I) has a structure of:

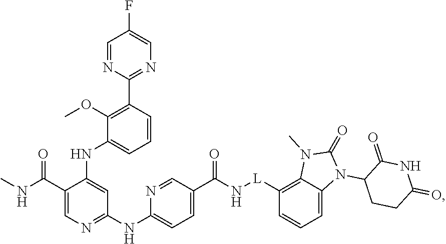

or a pharmaceutically acceptable salt thereof.

In some embodiments, Formula (I) has a structure of:

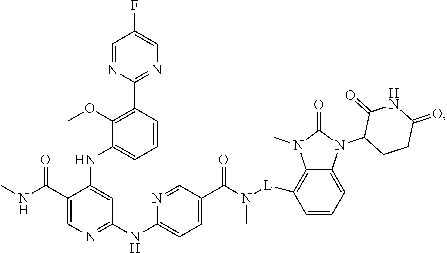

or a pharmaceutically acceptable salt thereof.

In certain embodiments, the present invention provides a compound of Formula I, wherein LBM is a cereblon E3 ubiquitin ligase binding moiety thereby forming a compound of Formula I-c:

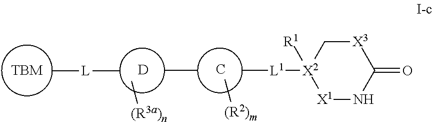

or a pharmaceutically acceptable salt thereof, wherein, L and TBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

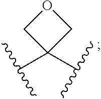 [SMILES: *C1(*)COC1]

X² is a carbon atom, nitrogen atom, or silicon atom;
X³ is a bivalent moiety selected from —CR₂—, —NR—, —O—, —S—, or —Si(R)₂—;
R¹ is absent, hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
Ring C is a mono- or bicyclic ring selected from

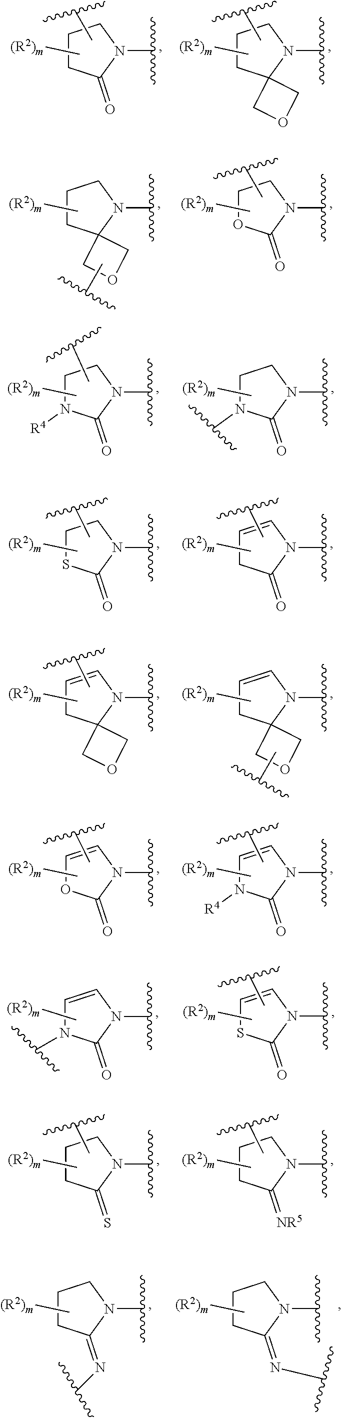

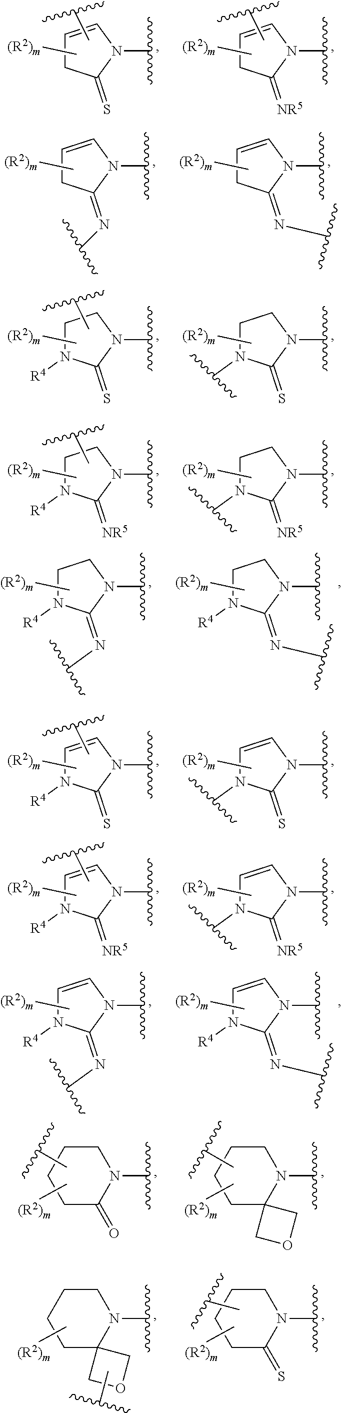

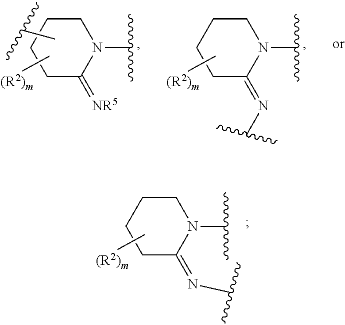

each of R² and R^{3a} is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring D is selected from a 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or 4;
n is 0, 1, 2, 3 or 4;
p is 0 or 1, wherein when p is 0, the bond connecting Ring C and Ring D is connected to

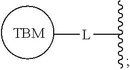

and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, a compound of Formula I-c above is provided as a compound of Formula I-c-1 or Formula I-c-2:

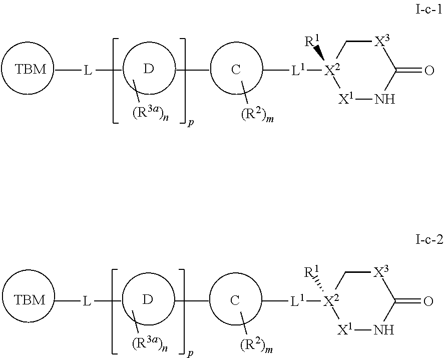

or a pharmaceutically acceptable salt thereof, wherein:
each of TBM, Ring C, Ring D, L, L¹, R¹, R², R^{3a}, X¹, X², X³, n, m, and p is as defined above.

In some embodiments, a compound of Formula I-c above is provided as a compound of Formula I-c-3:

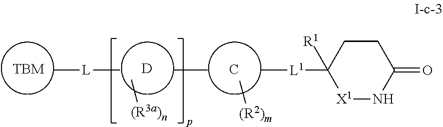

or a pharmaceutically acceptable salt thereof, wherein:
each of TBM, Ring C, Ring D, L, R¹, R², R^{3a}, X¹, n, m, and p is as defined above.

In certain embodiments, the present invention provides a compound of Formula I, wherein LBM is a cereblon E3 ubiquitin ligase binding moiety thereby forming a compound of Formula I-d:

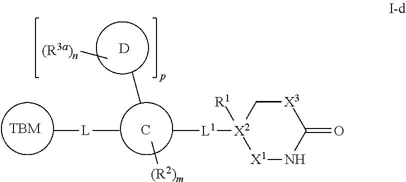

or a pharmaceutically acceptable salt thereof, wherein L and TBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

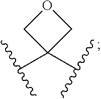 [SMILES: *C1(*)COC1]

X² is a carbon atom or silicon atom;
X³ is a bivalent moiety selected from —CR₂—, —NR—, —O—, —S—, or —Si(R)₂—;
R¹ is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
Ring C is a mono- or bicyclic ring selected from

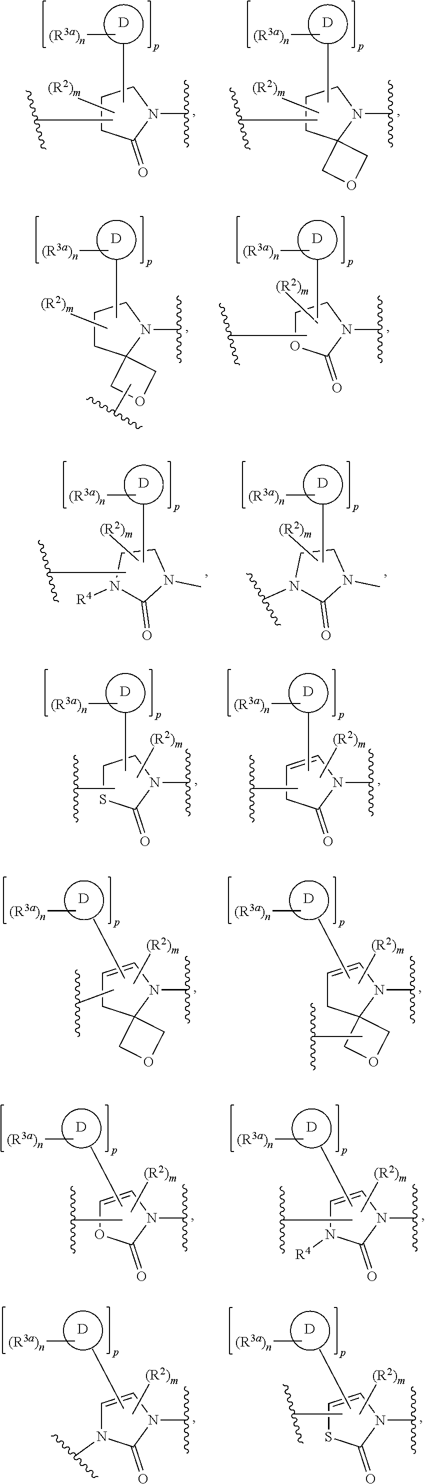

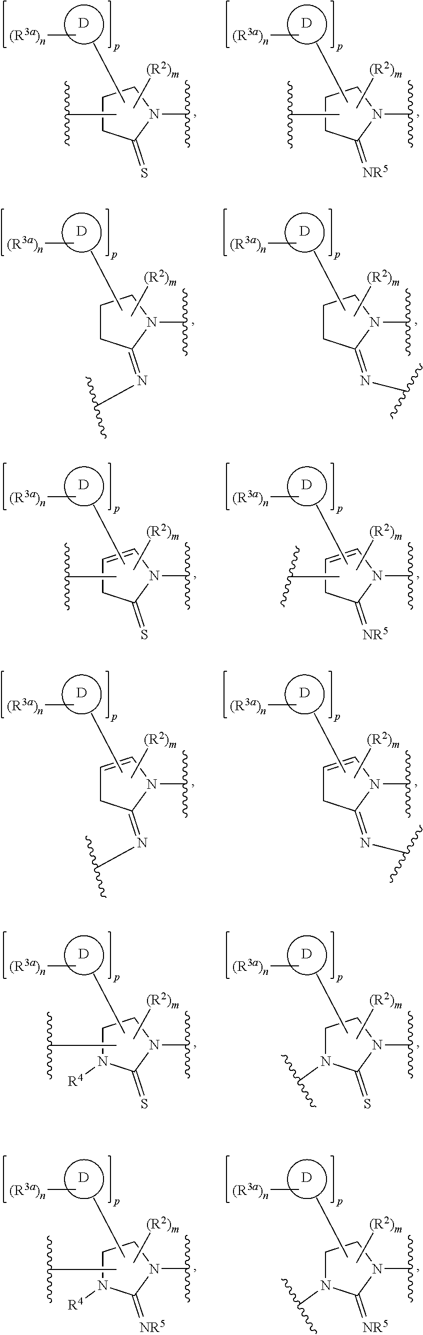

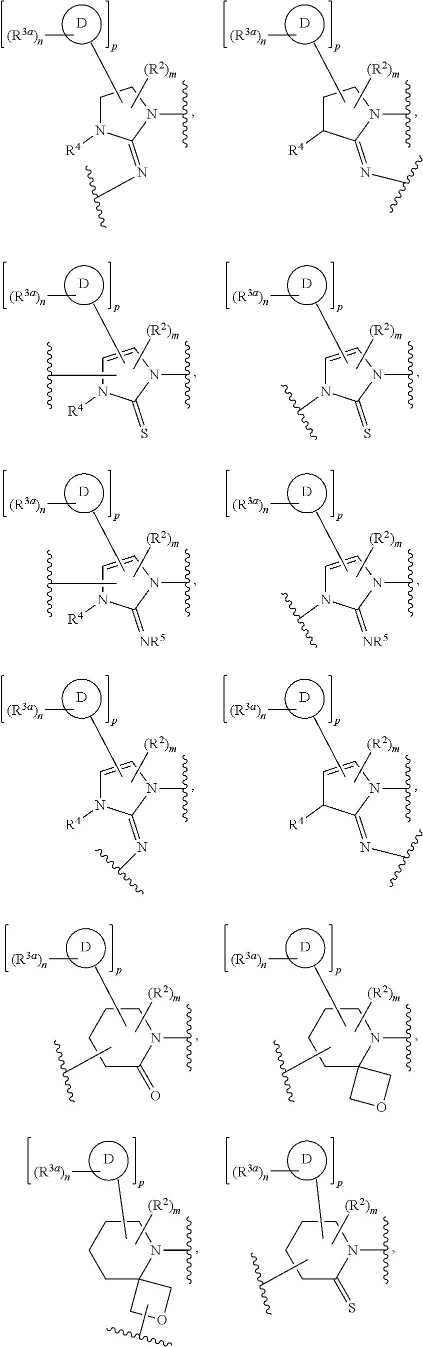

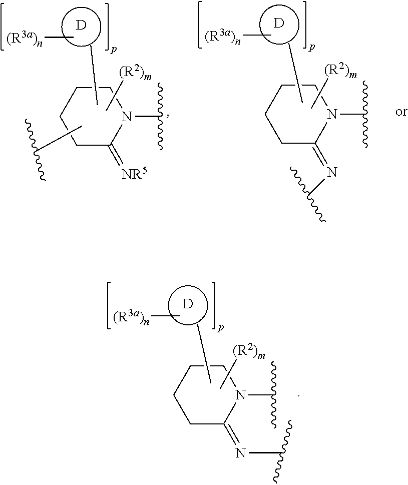

each or R² and R^{3a} is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring D is selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
each R⁴ is independently hydrogen, —R, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or 4;
n is 0, 1, 2, 3 or 4;
p is 0 or 1; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, a compound of Formula I-d above is provided as a compound of Formula I-d-1 or Formula I-d-2:

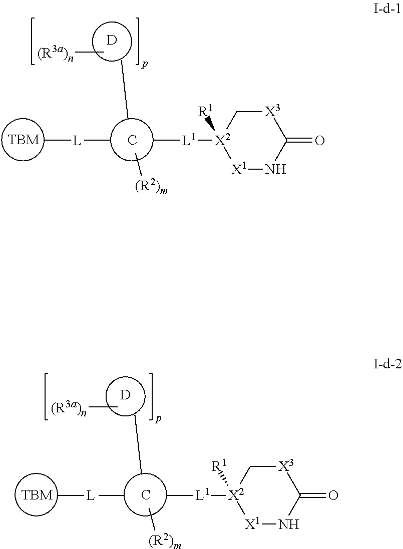

or a pharmaceutically acceptable salt thereof, wherein:
each of TBM, Ring C, Ring D, L, L¹, R¹, R², R^{3a}, X¹, X², X³, m, n, and p is as defined above.

In some embodiments, a compound of Formula I-d above is provided as a compound of Formula I-d-3:

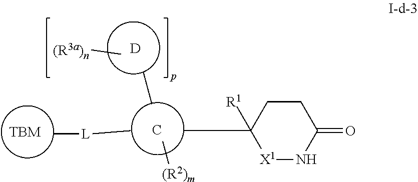

or a pharmaceutically acceptable salt thereof, wherein:
each of TBM, Ring C, Ring D, L, LR¹, R², R^{3a}, X¹, m, n, and p is as defined above.

In certain embodiments, the present invention provides a compound of Formula I, wherein LBM is a cereblon E3 ubiquitin ligase binding moiety thereby forming a compound of Formula I-e:

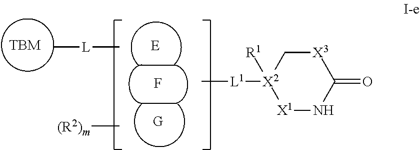

or a pharmaceutically acceptable salt thereof, wherein L and TBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

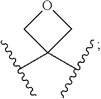 [SMILES: *C1(*)COC1]

X² is a carbon atom or silicon atom;
X³ is a bivalent moiety selected from —CR₂—, —NR—, —O—, —S—, or —Si(R)₂—;
R¹ is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂P, —NP(O)R₂, —N(R)P(O)(OR)₂, —N(R)P(O)(OR)(NR₂), —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring E, Ring F, and Ring G is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur, wherein each of Ring E, Ring F, and Ring G is independently and optionally further substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—; and
m is 0, 1, 2, 3, 4, 5, 6, 7, 8, 9, 10, 11, 12, 13, 14, 15, or 16.

Where a point of attachment of

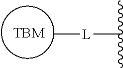

is depicted on Ring E, Ring F, or Ring G, it is intended, and one of ordinary skill in the art would appreciate, that the point of attachment of

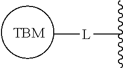

may be on any available carbon or nitrogen atom on Ring E, Ring F, or Ring G, including the ring to which Ring E or Ring G are fused to Ring F.

Where a point of attachment of —(R²)ₘ is depicted on Ring E, Ring F, or Ring G, it is intended, and one of ordinary skill in the art would appreciate, that the point of attachment of —(R²)ₘ may be at any available carbon or nitrogen atom on Ring E, Ring F, or Ring G including the carbon atom to which Ring E or Ring G are fused to Ring F.

Where a point of attachment of

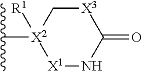

is depicted on Ring E, Ring F, or Ring G, it is intended, and one of ordinary skill in the art would appreciate, that the point of attachment of

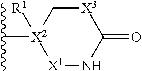

may be on any available carbon or nitrogen atom on Ring E, Ring F, or Ring G, including the carbon atom to which Ring E or Ring G are fused to Ring F.

In some embodiments, a compound of Formula I-e above is provided as a compound of Formula I-e-1 or Formula I-e-2:

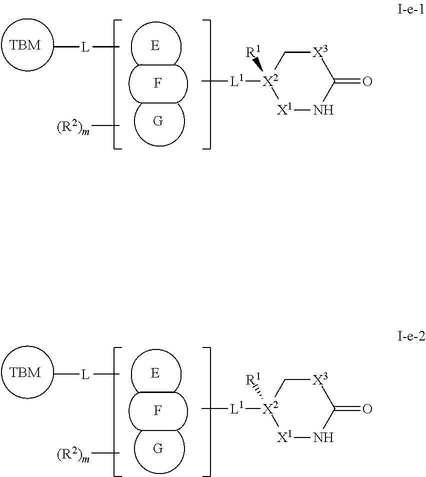

or a pharmaceutically acceptable salt thereof, wherein:
each of TBM, Ring E, Ring F, Ring G, L, L¹, R¹, R², X¹, X², X³, and m is as defined above.

In some embodiments, a compound of Formula I-e above is provided as a compound of Formula I-e-3:

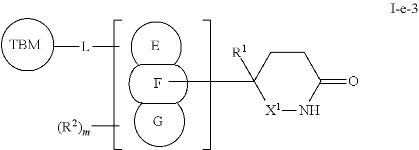

or a pharmaceutically acceptable salt thereof, wherein:
each of TBM, Ring E, Ring F, Ring G, L, R¹, R², X¹, and m is as defined above.

In certain embodiments, the present invention provides a compound of Formula I, wherein LBM is an E3 ubiquitin ligase (cereblon) binding moiety thereby forming a compound of Formula I-f:

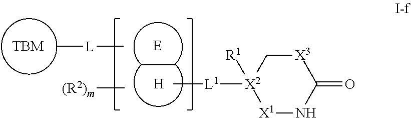

or a pharmaceutically acceptable salt thereof, wherein L and TBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

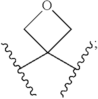 [SMILES: *C1(*)COC1]

X² is a carbon atom or silicon atom;
X³ is a bivalent moiety selected from —CR₂—, —NR—, —O—, —S—, or —Si(R)₂—;
R¹ is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂P, —NP(O)R₂, —N(R)P(O)(OR)₂, —N(R)P(O)(OR)(NR₂), —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring E is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
Ring H is a fused ring selected from a 7-9 membered saturated or partially unsaturated carbocyclyl or heterocyclyl ring with 1-3 heteroatoms independently selected from boron, nitrogen, oxygen, silicon, and sulfur, wherein Ring E is optionally further substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3, or 4.

Where a point of attachment of

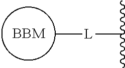

is depicted on Ring E or Ring H, it is intended, and one of ordinary skill in the art would appreciate, that the point of attachment of

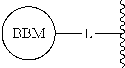

may be on any available carbon or nitrogen atom on Ring E or Ring H including the carbon atom to which Ring E and Ring H are fused.

Where a point of attachment of —(R²)ₘ is depicted on Ring E and Ring H, it is intended, and one of ordinary skill in the art would appreciate, that the point of attachment of —(R²)ₘ may be on any available carbon or nitrogen atom on Ring E or Ring H including the carbon atom to which Ring E and Ring H are fused.

Where a point of attachment of

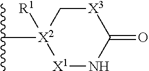

is depicted on Ring E and Ring H, it is intended, and one of ordinary skill in the art would appreciate, that the point of attachment of

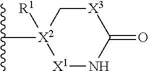

may be on any available carbon or nitrogen atom on Ring E or Ring H including the carbon atom to which Ring E and Ring H are fused.

In some embodiments, a compound of Formula I-f above is provided as a compound of Formula I-f-1 or Formula I-f-2:

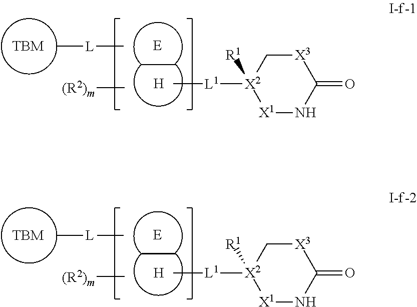

or a pharmaceutically acceptable salt thereof, wherein:
each of TBM, Ring E, Ring H, L, L¹, R¹, R², X¹, X², X³, and m is as defined above.

In some embodiments, a compound of Formula I-f above is provided as a compound of Formula I-f-3:

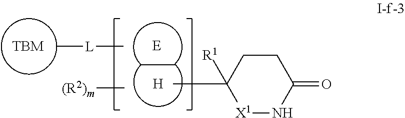

or a pharmaceutically acceptable salt thereof, wherein:
each of TBM, Ring E, Ring H, L, R¹, R², X¹, and m is as defined above.

In certain embodiments, the present invention provides a compound of Formula I, wherein LBM is an E3 ubiquitin ligase (cereblon) binding moiety thereby forming a compound of Formula I-g:

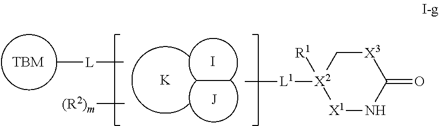

or a pharmaceutically acceptable salt thereof, wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

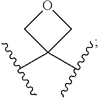 [SMILES: *C1(*)COC1]

X² is a carbon atom or silicon atom;
X³ is a bivalent moiety selected from —CR₂—, —NR—, —O—, —S—, or —Si(R)₂—;
R¹ is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring I and J is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
Ring K is a fused ring selected from a 6-12 membered saturated or partially unsaturated carbocyclyl or heterocyclyl ring with 1-3 heteroatoms independently selected from boron, nitrogen, oxygen, silicon, and sulfur, wherein Ring H is optionally further substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—; and
m is 0, 1, 2, 3, or 4.

Where a point of attachment of

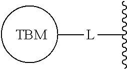

is depicted on Ring I, Ring J, and Ring K, it is intended, and one of ordinary skill in the art would appreciate, that the point of attachment of

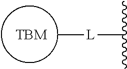

may be on any available carbon or nitrogen atom on Ring I, Ring J, or Ring K, including the carbon atom to which Ring I, Ring J, and Ring K are fused.

Where a point of attachment of —(R²)ₘ is depicted on Ring I, Ring J, and Ring K, it is intended, and one of ordinary skill in the art would appreciate, that the point of attachment of —(R²)ₘ may be on any available carbon or nitrogen atom on Ring I, Ring J, or Ring K, including the carbon atom to which Ring I, Ring J, and Ring K are fused.

Where a point of attachment of

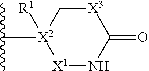

is depicted on Ring I, Ring J, and Ring K, it is intended, and one of ordinary skill in the art would appreciate, that the point of attachment of

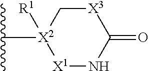

may be on any available carbon or nitrogen atom on Ring I, Ring J, or Ring K, including the carbon atom to which Ring I, Ring J, and Ring K are fused.

In some embodiments, a compound of Formula I-g above is provided as a compound of Formula I-g-1 or Formula I-g-2:

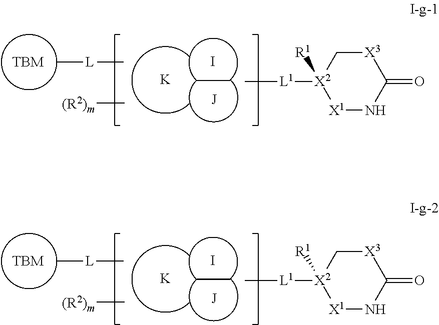

or a pharmaceutically acceptable salt thereof, wherein:
each of TBM, Ring I, Ring J, Ring K, L, L¹, R¹, R², X¹, X², X³, and m is as defined above.

In some embodiments, a compound of Formula I-g above is provided as a compound of Formula I-g-3:

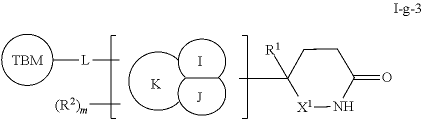

or a pharmaceutically acceptable salt thereof, wherein:
each of TBM, Ring I, Ring J, Ring K, L, R¹, R², X¹, and m is as defined above.

In certain embodiments, the present invention provides a compound of Formula I, wherein LBM is an E3 ubiquitin ligase (cereblon) binding moiety thereby forming a compound of Formula I-h-1 or I-h-2:

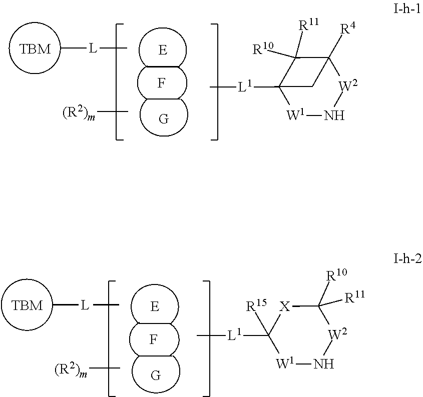

or a pharmaceutically acceptable salt thereof, wherein L and TBM are as defined above and described in embodiments herein, and wherein:
each R² is independently hydrogen, deuterium, —R, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring E, Ring F, and Ring G is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, and sulfur, or 5-membered heteroaryl with 1-4 heteroatoms independently selected from nitrogen, oxygen and sulfur, wherein each of Ring E, Ring F, and Ring G is independently and optionally further substituted with 1-2 oxo groups;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
m is 0, 1, 2, 3, 4, 5, 6, 7, 8, 9, 10, 11, 12, 13, 14, 15, or 16; and
R⁴, R¹⁰, R¹¹, R¹⁵, W¹, W², and X is as defined in WO 2019/099868, the entirety of each of which is herein incorporated by reference.

Where a point of attachment of

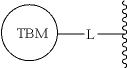

is depicted on Ring E, Ring F, or Ring G, it is intended, and one of ordinary skill in the art would appreciate, that the point of attachment of

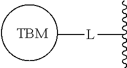

may be on any available carbon or nitrogen atom on Ring E, Ring F, or Ring G, including the ring to which Ring E or Ring G are fused to Ring F.

Where a point of attachment of —(R²)ₘ is depicted on Ring E, Ring F, or Ring G, it is intended, and one of ordinary skill in the art would appreciate, that the point of attachment of —(R²)ₘ may be at any available carbon or nitrogen atom on Ring E, Ring F, or Ring G including the carbon atom to which Ring E or Ring G are fused to Ring F.

Where a point of attachment of

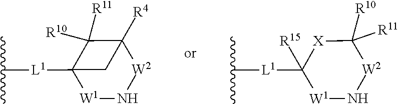

is depicted on Ring E, Ring F, or Ring G, it is intended, and one of ordinary skill in the art would appreciate, that the point of attachment of

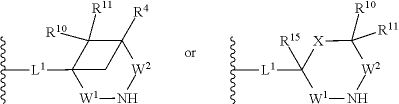

may be on any available carbon or nitrogen atom on Ring E, Ring F, or Ring G, including the carbon atom to which Ring E or Ring G are fused to Ring F.

As described above, in another aspect, the present invention provides a compound of Formula I, wherein said compound is a compound of Formula I-h-3:

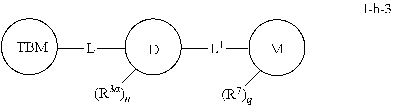

or a pharmaceutically acceptable salt thereof, wherein:
Ring M is selected from

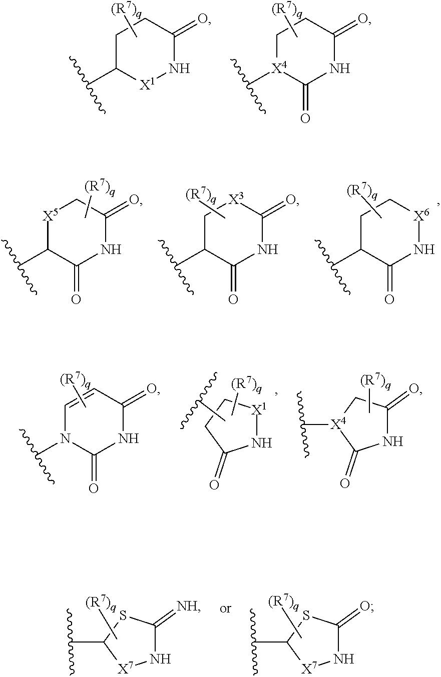

each of X¹, X⁶, and X⁷ is independently a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

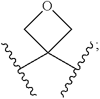 [SMILES: *C1(*)COC1]

each of X³ and X⁵ is independently a bivalent moiety selected from a covalent bond, —CR₂—, —NR—, —O—, —S—, or —SiR₂—;
X⁴ is a trivalent moiety selected from

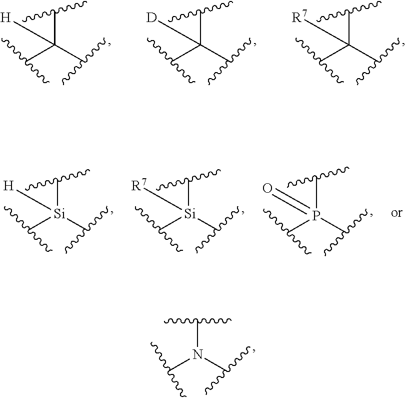

each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R^{3a} is independently hydrogen, deuterium, —R⁶, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁷ is independently hydrogen, deuterium, halogen, —CN, —OR, —SR,
—S(O)R, —S(O)₂R, —NR₂, —P(O)(OR)₂, —P(O)(NR₂)OR, —P(O)(NR₂)₂, —Si(OH)R₂, —Si(OH)₂R, —SiR₃, or an optionally substituted C₁₋₄ aliphatic; or
R⁷ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, and sulfur;
two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, and sulfur;
two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, and sulfur, or a 7-13 membered saturated, partially unsaturated, bridged heterocyclic ring, or a spiro heterocyclic ring having 1-3 heteroatoms, independently selected from boron, nitrogen, oxygen, silicon, and sulfur;
Ring D is selected from 6 to 10-membered aryl or heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, and sulfur, or 5-membered heteroaryl with 1-4 heteroatoms independently selected from nitrogen, oxygen and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
n is 0, 1, 2, 3, or 4; and
q is 0, 1, 2, 3, or 4.

As defined above and described herein, each of X¹, X⁶, and X⁷ is independently a bivalent moiety selected from a covalent bond, —CH₂—, —C(R)₂—, —C(O)—, —C(S)—, —CH(R)—, —CH(CF₃)—, —P(O)(OR)—, —P(O)(R)—, —P(O)(NR₂)—, —S(O)—, —S(O)₂—, or

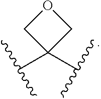 [SMILES: *C1(*)COC1]

In some embodiments, each of X¹, X⁶, and X⁷ is independently a covalent bond. In some embodiments, each of X¹, X⁶, and X⁷ is independently —CH₂—. In some embodiments, each of X¹, X⁶, and X⁷ is independently —CR₂—. In some embodiments, each of X¹, X⁶, and X⁷ is independently —C(O)—. In some embodiments, each of X¹, X⁶, and X⁷ is independently is —C(S)—. In some embodiments, each of X¹, X⁶, and X⁷ is independently —CH(R)—. In some embodiments, each of X¹, X⁶, and X⁷ is independently —CH(CF₃)—. In some embodiments, each of X¹, X⁶, and X⁷ is independently —P(O)(OR)—. In some embodiments, each of X¹, X⁶, and X⁷ is independently —P(O)(R)—. In some embodiments, each of X¹, X⁶, and X⁷ is independently —P(O)NR₂—. In some embodiments, each of X¹, X⁶, and X⁷ is independently —S(O)—. In some embodiments, each of X¹, X⁶, and X⁷ is independently —S(O)₂—. In some embodiments, each of X¹, X⁶, and X⁷ is independently

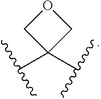 [SMILES: *C1(*)COC1]

In some embodiments, each of X¹, X⁶, and X⁷ is independently selected from those depicted in Table 1 below.

As defined above and described herein, X² is a carbon atom, nitrogen atom, or silicon atom. In some embodiments, X² is a carbon atom or silicon atom.

In some embodiments, X² is a carbon atom. In some embodiments, X² is a silicon atom. In some embodiments, X² is a nitrogen atom.

In some embodiments, X² is selected from those depicted in Table 1, below.

As defined above and described herein, each of X³ and X⁵ is independently a bivalent moiety selected from —CH₂—, —CR₂—, —NR—, —CF₂—, —CHF—, —S—, —CH(R)—, —SiR₂—, or —O—.

In some embodiments, each of X³ and X⁵ is independently —CH₂—. In some embodiments, each of X³ and X⁵ is independently —CR₂—. In some embodiments, each of X³ and X⁵ is independently —NR—. In some embodiments, each of X³ and X⁵ is independently —CF₂—. In some embodiments, each of X³ and X⁵ is independently —CHF—. In some embodiments, each of X³ and X⁵ is independently —S—. In some embodiments, each of X³ and X⁵ is independently —CH(R)—. In some embodiments, each of X³ and X⁵ is independently —SiR₂—. In some embodiments, each of X³ and X⁵ is independently —O—.

In some embodiments, each of X³ and X⁵ is independently selected from those depicted in Table 1 below.

As defined above and described herein, X⁴ is a trivalent moiety selected from

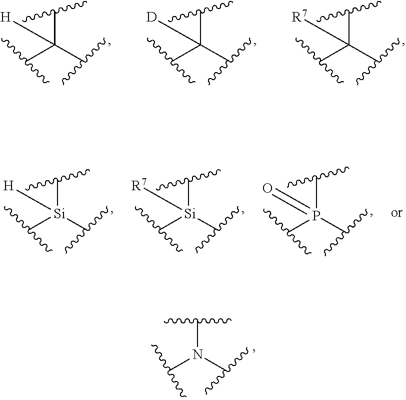

In some embodiments, X⁴ is

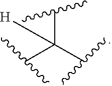 [SMILES: *C(*)*]

In some embodiments, X⁴ is

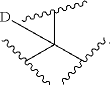 [SMILES: *C(*)(*)[2H]]

In some embodiments, X⁴ is

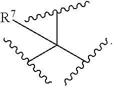

In some embodiments, X⁴ is

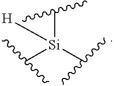 [SMILES: *[SiH](*)*]

In some embodiments, X⁴ is

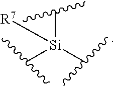

In some embodiments, X⁴ is

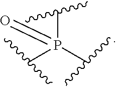 [SMILES: *P(*)(*)=O]

In some embodiments, X⁴ is

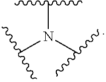 [SMILES: *N(*)*]

In some embodiments, X⁴ is selected from those depicted in Table 1 below.

As defined above and described herein, R¹ is hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)(R)₂, —Si(R)₃, an optionally substituted C₁₋₄ aliphatic, or R¹ and X¹ or X⁴ are taken together with their intervening atoms to form a 5-7 membered saturated, partially unsaturated, carbocyclic ring or heterocyclic ring having 1-3 heteroatoms, independently selected from nitrogen, oxygen, and sulfur.

In some embodiments, R¹ is hydrogen. In some embodiments, R¹ is deuterium. In some embodiments, R¹ is halogen. In some embodiments, R¹ is —CN. In some embodiments, R¹ is —OR. In some embodiments, R¹ is —SR. In some embodiments, R¹ is —S(O)R. In some embodiments, R¹ is —S(O)₂R. In some embodiments, R¹ is —NR₂. In some embodiments, R¹ is —P(O)(OR)₂. In some embodiments, R¹ is —P(O)(NR₂)OR. In some embodiments, R¹ is —P(O)(NR₂)₂. In some embodiments, R¹ is —Si(OH)₂R. In some embodiments, R¹ is —Si(OH)(R)₂. In some embodiments, R¹ is —Si(R)₃. In some embodiments, R¹ is an optionally substituted C₁₋₄ aliphatic. In some embodiments, R¹ and X¹ or X⁴ are taken together with their intervening atoms to form a 5-7 membered saturated, partially unsaturated, carbocyclic ring or heterocyclic ring having 1-3 heteroatoms, independently selected from nitrogen, oxygen, and sulfur.

In some embodiments, R¹ is selected from those depicted in Table 1, below.

As defined above and described herein, each R is independently hydrogen, deuterium, or an optionally substituted group selected from C₁₋₆ aliphatic, phenyl, a 4-7 membered saturated or partially unsaturated heterocyclic having 1-3 heteroatoms independently selected from boron, nitrogen, oxygen, silicon, and sulfur, and a 5-6 membered heteroaryl ring having 1-4 heteroatoms independently selected from boron, nitrogen, oxygen, silicon, and sulfur, or two R groups on the same nitrogen are taken together with their intervening atoms to form a 4-7 membered saturated, partially unsaturated, or heteroaryl ring having 0-3 heteroatoms, in addition to the nitrogen, independently selected from boron, nitrogen, oxygen, silicon, and sulfur.

In some embodiments, R is hydrogen. In some embodiments, R is deuterium. In some embodiments, R is optionally substituted C₁₋₆ aliphatic. In some embodiments, R is optionally substituted phenyl. In some embodiments, R is optionally substituted 4-7 membered saturated or partially unsaturated heterocyclic having 1-3 heteroatoms independently selected from boron, nitrogen, oxygen, silicon, and sulfur. In some embodiments, R is optionally substituted 5-6 membered heteroaryl ring having 1-4 heteroatoms independently selected from boron, nitrogen, oxygen, silicon, and sulfur. In some embodiments, two R groups on the same nitrogen are taken together with their intervening atoms to form a 4-7 membered saturated, partially unsaturated, or heteroaryl ring having 0-3 heteroatoms, in addition to the nitrogen, independently selected from boron, nitrogen, oxygen, silicon, and sulfur.

In some embodiments, R is selected from those depicted in Table 1, below.

As defined above and described herein, each of R² and R^{3a} is independently hydrogen, deuterium, —R⁶, halogen, —CN, —NO₂, —OR, —Si(OH)₂R, —Si(OH)R₂, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R.

In some embodiments, R² and/or R^{3a} is hydrogen. In some embodiments, R² and/or R^{3a} is deuterium. In some embodiments, R² and/or R^{3a} is —R⁶. In some embodiments, R² and/or R^{3a} is halogen. In some embodiments, R² and/or R^{3a} is —CN. In some embodiments, R² and/or R^{3a} is —NO₂. In some embodiments, R² and/or R^{3a} is —OR. In some embodiments, R² and/or R^{3a} is —Si(OH)₂R. In some embodiments, R² and/or R^{3a} is —Si(OH)R₂. In some embodiments, R² and/or R^{3a} is —SR. In some embodiments, R² and/or R^{3a} is —NR₂. In some embodiments, R² and/or R^{3a} is —SiR₃. In some embodiments, R² and/or R^{3a} is —S(O)₂R. In some embodiments, R² and/or R^{3a} is —S(O)₂NR₂. In some embodiments, R² and/or R^{3a} is —S(O)R. In some embodiments, R² and/or R^{3a} is —C(O)R. In some embodiments, R² and/or R^{3a} is —C(O)OR. In some embodiments, R² and/or R^{3a} is —C(O)NR₂. In some embodiments, R² and/or R^{3a} is —C(O)N(R)OR. In some embodiments, R² and/or R^{3a} is —C(R)₂N(R)C(O)R. In some embodiments, R² and/or R^{3a} is —C(R)₂N(R)C(O)NR₂. In some embodiments, R² and/or R^{3a} is —OC(O)R. In some embodiments, R² and/or R^{3a} is —OC(O)NR₂. In some embodiments, R² and/or R^{3a} is —OP(O)R₂. In some embodiments, R² and/or R^{3a} is —OP(O)(OR)₂. In some embodiments, R² and/or R^{3a} is —OP(O)(OR)NR₂. In some embodiments, R² and/or R^{3a} is —OP(O)(NR₂)₂—. In some embodiments, R² and/or R^{3a} is —N(R)C(O)OR. In some embodiments, R² and R^{3a} is independently —N(R)C(O)R. In some embodiments, R² and/or R^{3a} is —N(R)C(O)NR₂. In some embodiments, R² and/or R^{3a} is —NP(O)R₂. In some embodiments, R² and/or R^{3a} is —N(R)P(O)(OR)₂. In some embodiments, R² and/or R^{3a} is —N(R)P(O)(OR)NR₂. In some embodiments, R² and/or R^{3a} is —N(R)P(O)(NR₂)₂. In some embodiments, R² and/or R^{3a} is —N(R)S(O)₂R.

In some embodiments, R² and R^{3a} is independently —OH. In some embodiments, R² and R^{3a} is independently —NH₂. In some embodiments, R² and R^{3a} is independently —CH₂NH₂. In some embodiments, R² and R^{3a} is independently —CH₂NHCOMe. In some embodiments, R² and R^{3a} is independently —CH₂NHCONHMe. In some embodiments, R² and R^{3a} is independently —NHCOMe. In some embodiments, R² and R^{3a} is independently —NHCONHEt. In some embodiments, R² and R^{3a} is independently —SiMe₃. In some embodiments, R² and R^{3a} is independently —SiMe₂OH. In some embodiments, R² and R^{3a} is independently —SiMe(OH)₂. In some embodiments R² and/or R^{3a} is

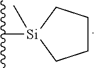 [SMILES: *[Si]1(C)CCCC1]

In some embodiments, ² and/or R^{3a} is Br. In some embodiments, R² and/or R^{3a} is Cl. In some embodiments, R² and/or R^{3a} is F. In some embodiments, R² and/or R^{3a} is Me. In some embodiments, R² and/or R^{3a} is —NHMe. In some embodiments, R² and/or R^{3a} is —NMe₂. In some embodiments, R² and/or R^{3a} is —NHCO₂Et. In some embodiments, R² and/or R^{3a} is —CN. In some embodiments, R² and/or R^{3a} is —CH₂Ph. In some embodiments, R² and/or R^{3a} is —NHCO₂tBu. In some embodiments, R² and/or R^{3a} is —CO₂tBu. In some embodiments, R² and/or R^{3a} is —OMe. In some embodiments, R² and/or R^{3a} is —CF₃.

In some embodiments, R² and R^{3a} are selected from those depicted in Table 1, below.

As defined above and described herein, R³ is hydrogen, deuterium, halogen, —CN, —NO₂, —OR, —NR₂, —SR, —S(O)₂R, —S(O)₂NR₂, —S(O)R, —C(O)R, —C(O)OR, —C(O)NR₂, —C(O)NR(OR), —OC(O)R, —OC(O)NR₂, —OP(O)(OR)₂, —OP(O)(NR₂)₂, —OP(O)(OR)NR₂, —N(R)C(O)R, —N(R)C(O)OR, —N(R)C(O)NR₂, —N(R)S(O)₂R, —N(R)S(O)₂NR₂, —N(R)P(O)(OR)₂, —N(R)P(O)(OR)NR₂, —P(O)(OR)₂, —P(O)(NR₂)OR, —P(O)(NR₂)₂, —Si(OH)₂R, —Si(OH)(R)₂, or —Si(R)₃.

In some embodiments, R³ is hydrogen. In some embodiments, R³ is deuterium. In some embodiments, R³ is halogen. In some embodiments, R³ is —CN. In some embodiments, R³ is —NO₂. In some embodiments, R³ is —OR. In some embodiments, R³ is —NR₂. In some embodiments, R³ is —SR. In some embodiments, R³ is —S(O)₂R. In some embodiments, R³ is —S(O)₂NR₂. In some embodiments, R³ is —S(O)R. In some embodiments, R³ is —C(O)R. In some embodiments, R³ is —C(O)OR. In some embodiments, R³ is —C(O)NR₂. In some embodiments, R³ is —C(O)NR(OR). In some embodiments, R³ is —OC(O)R. In some embodiments, R³ is —OC(O)NR₂. In some embodiments, R³ is —OP(O)(OR)₂. In some embodiments, R³ is —OP(O)(NR₂)₂. In some embodiments, R³ is —OP(O)(OR)NR₂. In some embodiments, R³ is —N(R)C(O)R. In some embodiments, R³ is —N(R)C(O)OR. In some embodiments, R³ is —N(R)C(O)NR₂. In some embodiments, R³ is —N(R)S(O)₂R. In some embodiments, R³ is —N(R)S(O)₂NR₂. In some embodiments, R³ is —N(R)P(O)(OR)₂. In some embodiments, R³ is —N(R)P(O)(OR)NR₂. In some embodiments, R³ is —P(O)(OR)₂. In some embodiments, R³ is —P(O)(NR₂)OR. In some embodiments, R³ is —P(O)(NR₂)₂. In some embodiments, R³ is —Si(OH)₂R. In some embodiments, R³ is —Si(OH)(R)₂. In some embodiments, R³ is —Si(R)₃.

In some embodiments, R³ is methyl. In some embodiments, R³ is —OCH₃. In some embodiments, R³ is chloro.

In some embodiments, R³ is selected from those depicted in Table 1, below.

As defined above and described herein, each R⁴ is independently hydrogen, deuterium, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —P(O)(OR)₂, —P(O)(NR₂)OR, or —P(O)(NR₂)₂.

In some embodiments, R⁴ is hydrogen. In some embodiments, R⁴ is —R⁶. In some embodiments, R⁴ is halogen. In some embodiments, R⁴ is —CN. In some embodiments, R⁴ is —NO₂. In some embodiments, R⁴ is —OR. In some embodiments, R⁴ is —SR. In some embodiments, R⁴ is —NR₂. In some embodiments, R⁴ is —S(O)₂R. In some embodiments, R⁴ is —S(O)₂NR₂. In some embodiments, R⁴ is —S(O)R. In some embodiments, R⁴ is —C(O)R. In some embodiments, R⁴ is —C(O)OR. In some embodiments, R⁴ is —C(O)NR₂. In some embodiments, R⁴ is —C(O)N(R)OR. In some embodiments, R⁴ is —OC(O)R. In some embodiments, R⁴ is —OC(O)NR₂. In some embodiments, R⁴ is —N(R)C(O)OR. In some embodiments, R⁴ is —N(R)C(O)R. In some embodiments, R⁴ is —N(R)C(O)NR₂. In some embodiments, R⁴ is —N(R)S(O)₂R. In some embodiments, R⁴ is —P(O)(OR)₂. In some embodiments, R⁴ is —P(O)(NR₂)OR. In some embodiments, R⁴ is —P(O)(NR₂)₂.

In some embodiments, R⁴ is methyl. In some embodiments, R⁴ is ethyl. In some embodiments, R⁴ is cyclopropyl.

In some embodiments, R⁴ is selected from those depicted in Table 1, below.

As defined above and described herein, R⁵ is hydrogen, deuterium, an optionally substitute C₁₋₄ aliphatic, or —CN.

In some embodiments, R⁵ is hydrogen. In some embodiments, R⁵ is deuterium. In some embodiments, R⁵ is an optionally substituted C₁₋₄ aliphatic. In some embodiments, R⁵ is —CN.

In some embodiments, R⁵ is selected from those depicted in Table 1, below.

As defined above and described herein, each R⁶ is independently an optionally substituted group selected from C₁₋₆ aliphatic, phenyl, a 4-7 membered saturated or partially unsaturated heterocyclic ring having 1-3 heteroatoms independently selected from boron, nitrogen, oxygen, silicon, and sulfur, and a 5-6 membered heteroaryl ring having 1-4 heteroatoms independently selected from boron, nitrogen, oxygen, silicon, and sulfur.

In some embodiments, R⁶ is an optionally substituted C₁₋₆ aliphatic. In some embodiments, R⁶ is an optionally substituted phenyl. In some embodiments, R⁶ is an optionally substituted 4-7 membered saturated or partially unsaturated heterocyclic ring having 1-3 heteroatoms independently selected from boron, nitrogen, oxygen, silicon, and sulfur. In some embodiments, R⁶ is an optionally substituted 5-6 membered heteroaryl ring having 1-4 heteroatoms independently selected from boron, nitrogen, oxygen, silicon, and sulfur.

In some embodiments, R⁶ is selected from those depicted in Table 1, below.

As defined generally above, each R⁷ is independently hydrogen, deuterium, halogen, —CN, —OR, —SR, —S(O)R, —S(O)₂R, —N(R)₂, —P(O)(R)₂, —P(O)(OR)₂, —P(O)(NR₂)OR, —P(O)(NR₂)₂, —Si(OH)R₂, —Si(OH)₂R, —SiR₃, or an optionally substituted C₁₋₄ aliphatic, or R¹ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, and sulfur, or two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, and sulfur, or two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, and sulfur, or a 7-13 membered saturated, partially unsaturated, bridged heterocyclic ring, or a spiro heterocyclic ring having 1-3 heteroatoms, independently selected from boron, nitrogen, oxygen, silicon, and sulfur.

In some embodiments, R⁷ is hydrogen. In some embodiments, R⁷ is deuterium. In some embodiments, R⁷ is halogen. In some embodiments, R⁷ is —CN. In some embodiments, R⁷ is —OR. In some embodiments, R⁷ is —SR. In some embodiments, R⁷ is —S(O)R. In some embodiments, R⁷ is —S(O)₂R. In some embodiments, R⁷ is —NR₂. In some embodiments, R⁷ is —Si(R)₃. In some embodiments, R⁷ is —P(O)(R)₂. In some embodiments, R⁷ is —P(O)(OR)₂. In some embodiments, R⁷ is —P(O)(NR₂)OR. In some embodiments, R⁷ is —P(O)(NR₂)₂. In some embodiments, R⁷ is —Si(OH)R₂. In some embodiments, R⁷ is —Si(OH)₂R. In some embodiments, R⁷ is an optionally substituted C₁₋₄ aliphatic. In some embodiments, R⁷ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, and sulfur. In some embodiments, two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, and sulfur. In some embodiments, two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, and sulfur. In some embodiments, two R⁷ groups on adjacent carbon atoms are optionally taken together with their intervening atoms to form a 7-13 membered saturated, partially unsaturated, bridged heterocyclic ring, or a spiro heterocyclic ring having 1-3 heteroatoms, independently selected from boron, nitrogen, oxygen, silicon, and sulfur.

In some embodiments, R⁷ is selected from hydrogen, halogen, —CN, —OR, —NR₂, or C₁₋₄ alkyl. In some embodiments, R⁷ is selected from hydrogen, halogen, —CN, or C₁₋₄ alkyl. In some embodiments, R⁷ is fluoro. In some embodiments, two R⁷ groups on the same carbon are optionally taken together with their intervening atoms to form a 3- or 4-membered spiro fused ring.

In some embodiments, R⁷ is selected from those depicted in Table 1 below.

As defined above and described herein, Ring A is a bi- or tricyclic ring selected from

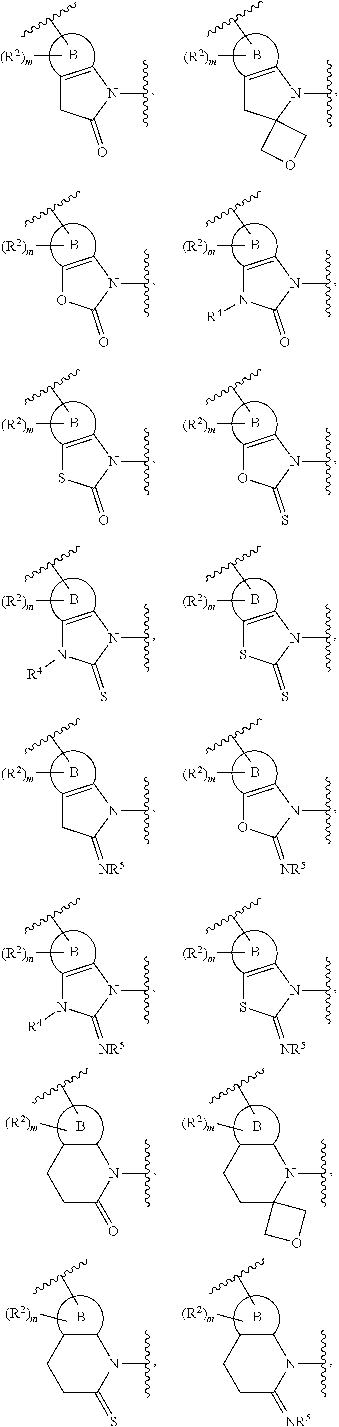

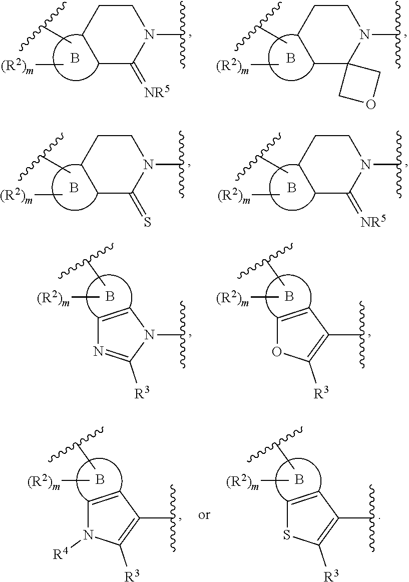

In some embodiments, Ring A is

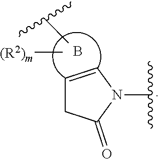

In some embodiments, Ring A is

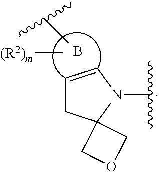

In some embodiments, Ring A is

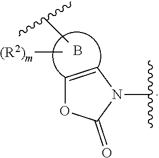

In some embodiments, Ring A is

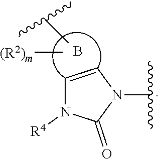

In some embodiments, Ring A is

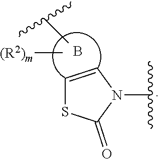

In some embodiments, Ring A is

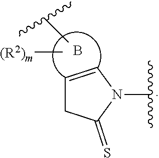

In some embodiments, Ring A is

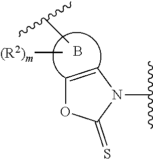

In some embodiments, Ring A is

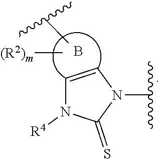

In some embodiments, Ring A is

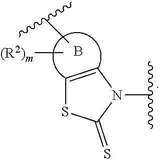

In some embodiments, Ring A is

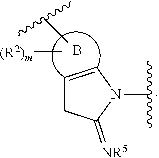

In some embodiments, Ring A is

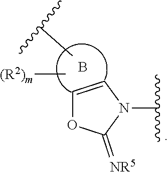

In some embodiments, Ring A is

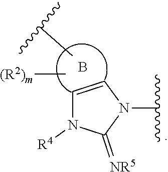

In some embodiments, Ring A is

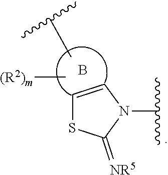

In some embodiments, Ring A is

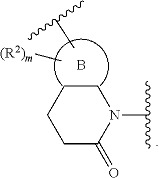

In some embodiments, Ring A is

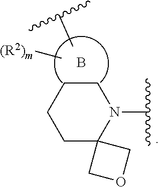

In some embodiments, Ring A is

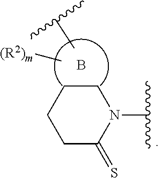

In some embodiments, Ring A is

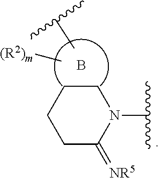

In some embodiments, Ring A is

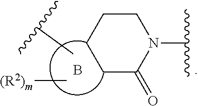

In some embodiments, Ring A is

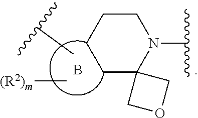

In some embodiments, Ring A is

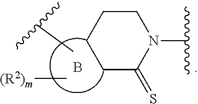

In some embodiments, Ring A is

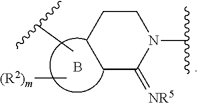

In some embodiments, Ring A is

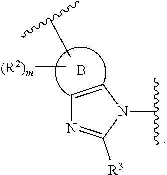

In some embodiments, Ring A is

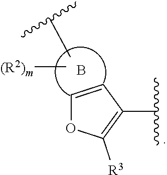

In some embodiments, Ring A is

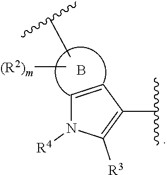

In some embodiments, Ring A is

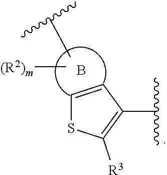

In some embodiments, Ring A is

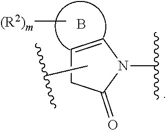

In some embodiments, Ring A is

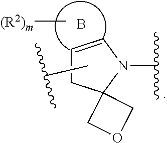

In some embodiments, Ring A is

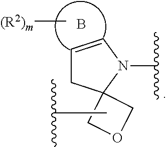

In some embodiments, Ring A is

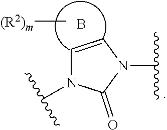

In some embodiments, Ring A is

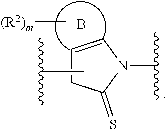

In some embodiments, Ring A is

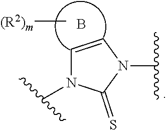

In some embodiments, Ring A is

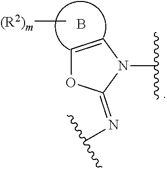

In some embodiments, Ring A is

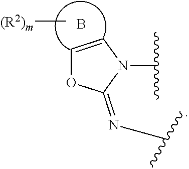

In some embodiments, Ring A is

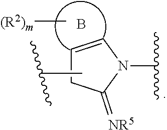

In some embodiments, Ring A is

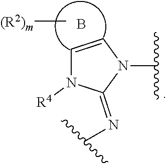

In some embodiments, Ring A is

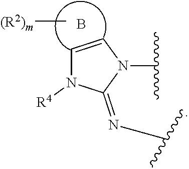

In some embodiments, Ring A is

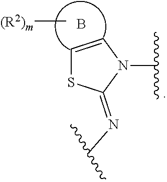

In some embodiments, Ring A is

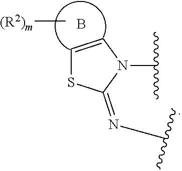

In some embodiments, Ring A is

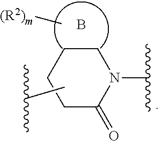

In some embodiments, Ring A is

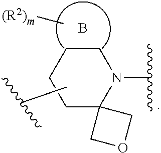

In some embodiments, Ring A is

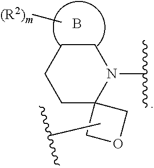

In some embodiments, Ring A is

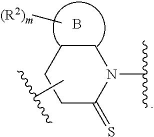

In some embodiments, Ring A is

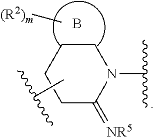

In some embodiments, Ring A is

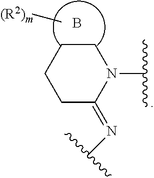

In some embodiments, Ring A is

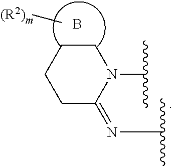

In some embodiments, Ring A is

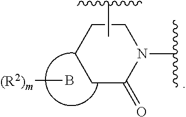

In some embodiments, Ring A is

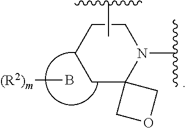

In some embodiments, Ring A is

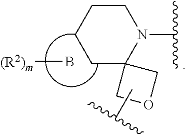

In some embodiments, Ring A is

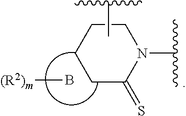

In some embodiments, Ring A is

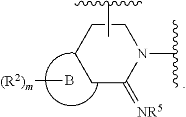

In some embodiments, Ring A is

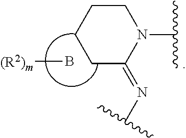

In some embodiments, Ring A is

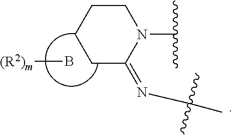

In some embodiments, Ring A is

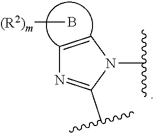

In some embodiments, Ring A is

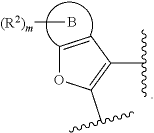

In some embodiments, Ring A is

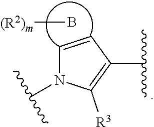

In some embodiments, Ring A is

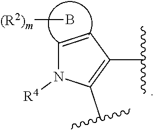

In some embodiments, Ring A is

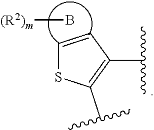

In some embodiments, Ring A is selected from those depicted in Table 1, below.

As defined above and described herein, Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;

In some embodiments, Ring B is a fused 6-membered aryl. In some embodiments, Ring B is a fused 6-membered heteroaryl containing 1-4 heteroatoms independently selected from nitrogen, oxygen, and sulfur. In some embodiments, Ring B is a fused 5 to 7-membered saturated or partially unsaturated carbocyclyl. In some embodiments, Ring B is fused 5 to 7-membered saturated or partially saturated heterocyclyl with 1-3 heteroatoms independently selected from boron, nitrogen, oxygen, silicon, and sulfur. In some embodiments, Ring B is fused 5-membered heteroaryl with 1-4 heteroatoms independently selected from boron, nitrogen, oxygen, silicon, and sulfur.

In some embodiments, Ring B is

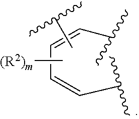

In some embodiments, Ring B is

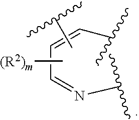

In some embodiments, Ring B is

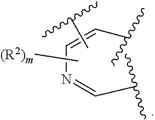

In some embodiments, Ring B is selected from those depicted in Table 1, below.

As defined above and described herein, Ring C is a mono- or bicyclic ring selected from

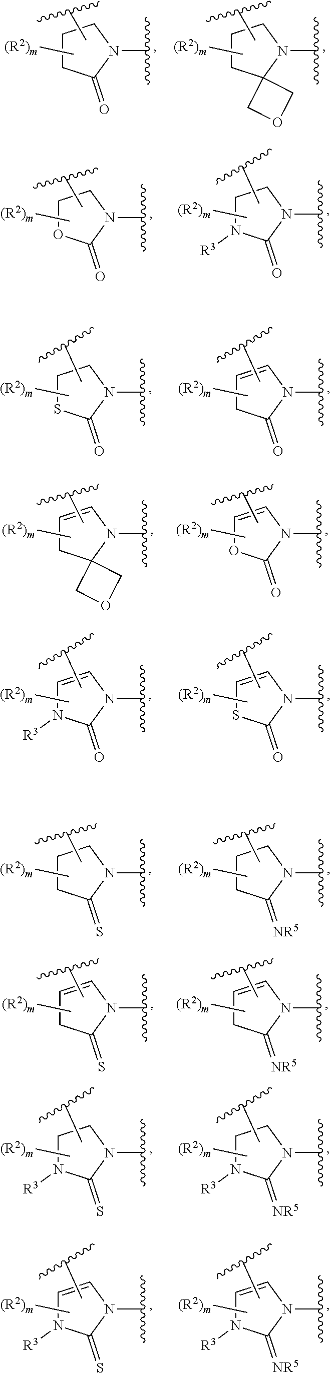

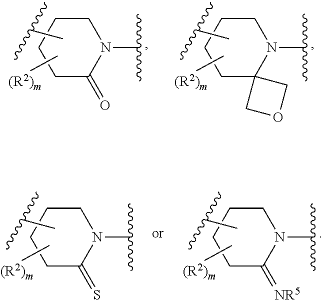

In some embodiments, Ring C is

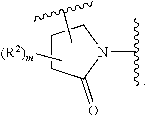

In some embodiments, Ring C is

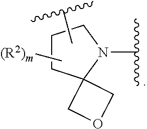

In some embodiments, Ring C is

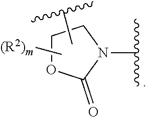

In some embodiments, Ring C is

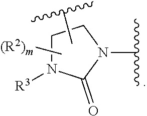

In some embodiments, Ring C is

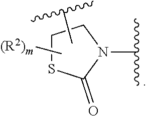

In some embodiments, Ring C is

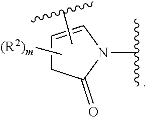

In some embodiments, Ring C is

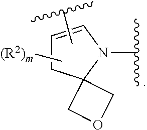

In some embodiments, Ring C is

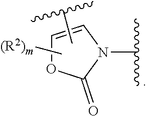

In some embodiments, Ring C is

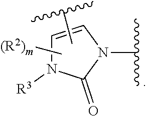

In some embodiments, Ring C is

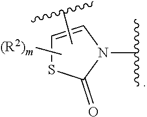

In some embodiments, Ring C is

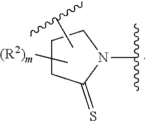

In some embodiments, Ring C is

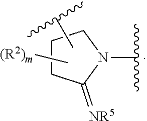

In some embodiments, Ring C is

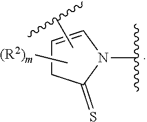

In some embodiments, Ring C is

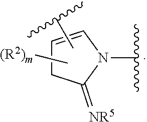

In some embodiments, Ring C is

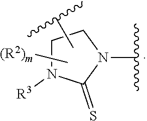

In some embodiments, Ring C is

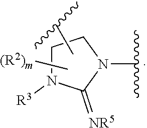

In some embodiments, Ring C is

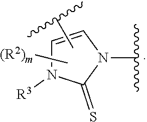

In some embodiments, Ring C is

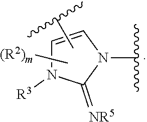

In some embodiments, Ring C is

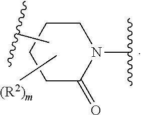

In some embodiments, Ring C is

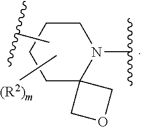

In some embodiments, Ring C is

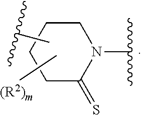

In some embodiments, Ring C is

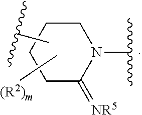

In some embodiments, Ring C is

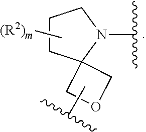

In some embodiments, Ring C is

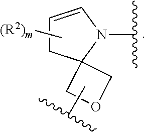

In some embodiments, Ring C is

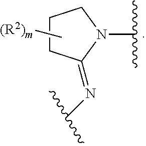

In some embodiments, Ring C is

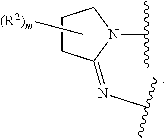

In some embodiments, Ring C is

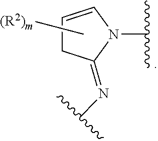

In some embodiments, Ring C is

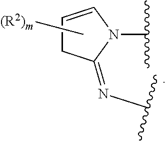

In some embodiments, Ring C is

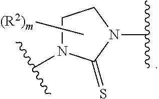

In some embodiments, Ring C is

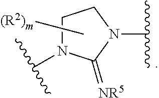

In some embodiments, Ring C is

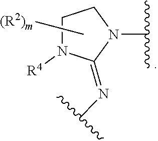

In some embodiments, Ring C is

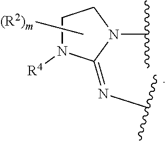

In some embodiments, Ring C is

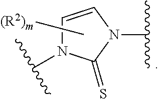

In some embodiments, Ring C is

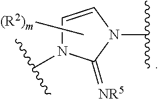

In some embodiments, Ring C is

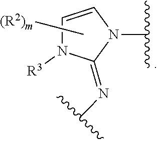

In some embodiments, Ring C is

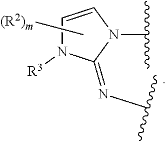

In some embodiments, Ring C is

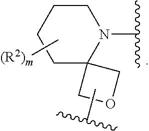

In some embodiments, Ring C is

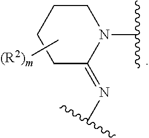

In some embodiments, Ring C is

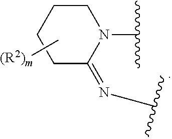

In some embodiments Ring C is a mono- or bicyclic ring selected from

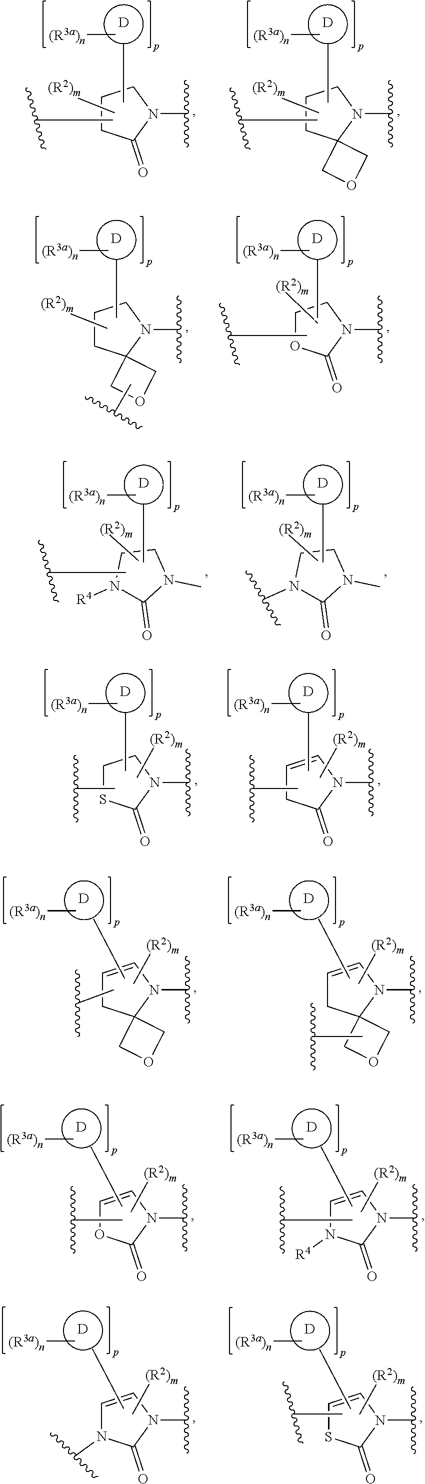

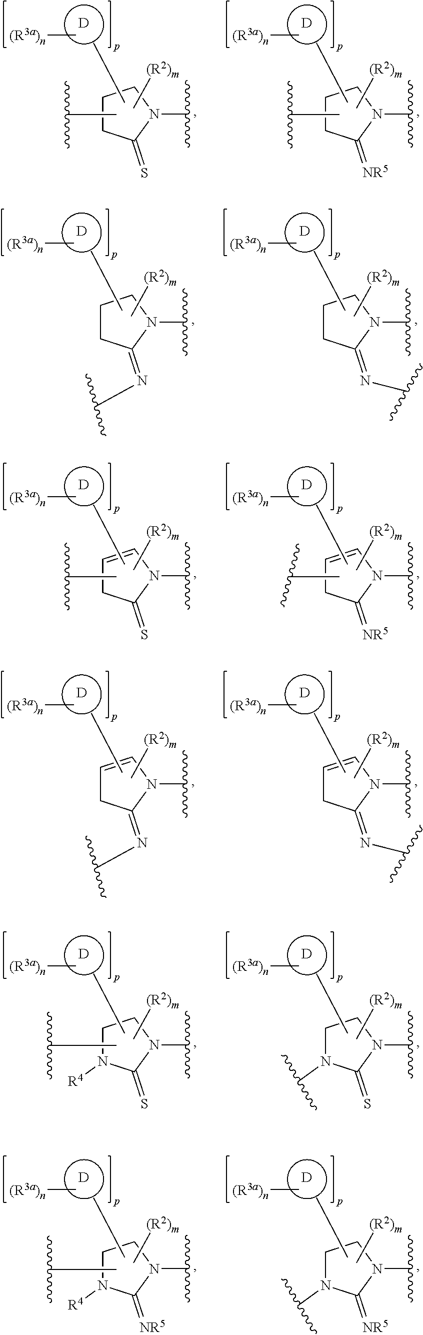

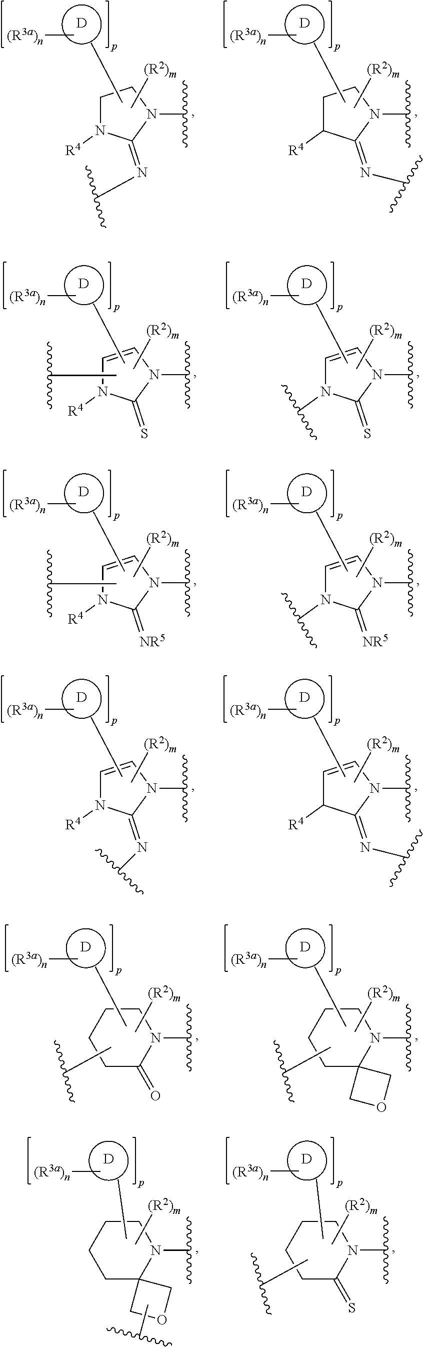

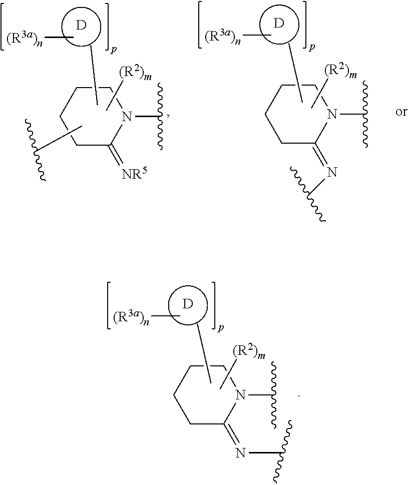

In some embodiments, Ring C is selected from

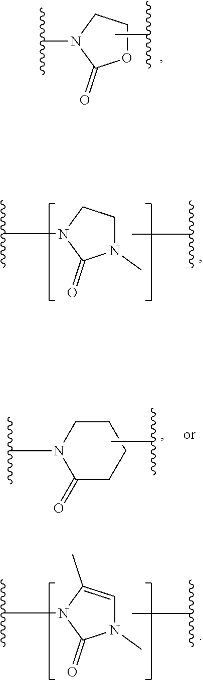

In some embodiments, Ring C is selected from

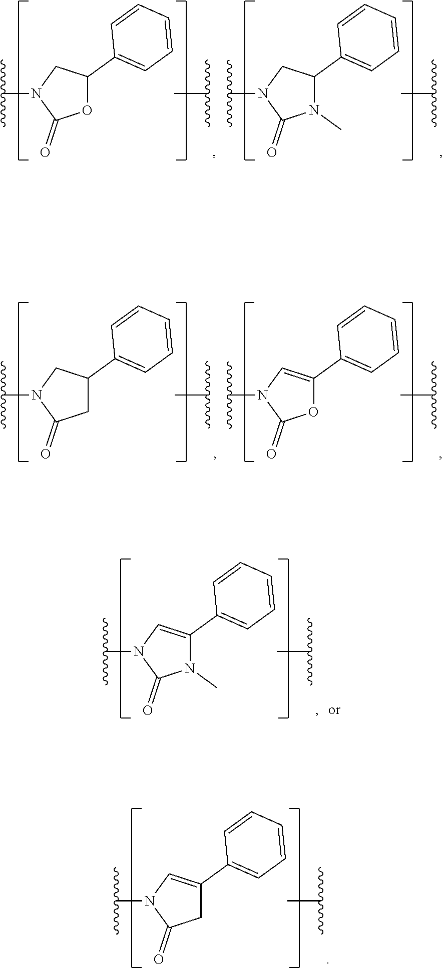

In some embodiments, Ring C is selected from those depicted in Table 1, below.

As defined above and described herein, Ring D is a ring selected from a 6 to 10-membered aryl or heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, and sulfur, or 5-membered heteroaryl with 1-4 heteroatoms independently selected from nitrogen, oxygen and sulfur;

In some embodiments, Ring D is a 6 to 10-membered aryl. In some embodiments, Ring D is a 6 to 10-membered heteroaryl containing 1-4 heteroatoms independently selected from nitrogen, oxygen, and sulfur. In some embodiments, Ring D is a 5 to 7-membered saturated or partially unsaturated carbocyclyl. In some embodiments, Ring D is 5 to 7-membered saturated or partially saturated heterocyclyl with 1-3 heteroatoms independently selected from boron, nitrogen, oxygen, silicon, and sulfur. In some embodiments, Ring D is 5-membered heteroaryl with 1-4 heteroatoms independently selected from boron, nitrogen, oxygen, silicon, and sulfur.

In some embodiments, Ring D is quinoline. In some embodiments, Ring D is isoquinoline. In some embodiments, Ring D is imidazo[1,2-a]pyridine.

In some embodiments, Ring D is selected from those depicted in Table 1 below.

As defined above and described herein, each of Ring E, Ring F, and Ring G is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur, wherein each of Ring E, Ring F, and Ring G is independently and optionally further substituted with 1-2 oxo groups.

In some embodiments, one or more of Ring E, Ring F, and Ring G is a 6-membered aryl. In some embodiments, one or more of Ring E, Ring F, and Ring G is a 6-membered heteroaryl containing 1-4 heteroatoms independently selected from nitrogen, oxygen, and sulfur. In some embodiments, one or more of Ring E, Ring F, and Ring G is a 5 to 7-membered saturated or partially unsaturated carbocyclyl. In some embodiments, one or more of Ring E, Ring F, and Ring G is independently a fused ring selected from a 5 to 7-membered saturated or partially unsaturated heterocyclyl with 1-3 heteroatoms independently selected from boron, nitrogen, oxygen, silicon, and sulfur. In some embodiments, one or more of Ring E, Ring F, and Ring G is a 5-membered heteroaryl with 1-4 heteroatoms independently selected from nitrogen, oxygen and sulfur. In some embodiments, one or more of Ring E, Ring F, and Ring G is and optionally further substituted with 1-2 oxo groups.

In some embodiments, Ring E, Ring F, and Ring G are selected from those depicted in Table 1, below.

As defined above and described herein, Ring H is a ring selected from a 7-9 membered saturated or partially unsaturated carbocyclyl or heterocyclyl ring with 1-3 heteroatoms independently selected from boron, nitrogen, oxygen, silicon, and sulfur, wherein Ring E is optionally further substituted with 1-2 oxo groups.

In some embodiments, Ring H is a ring selected from a 7-9 membered saturated or partially unsaturated carbocyclyl or heterocyclyl ring with 1-3 heteroatoms independently selected from boron, nitrogen, oxygen, silicon, and sulfur, wherein Ring H is optionally further substituted with 1-2 oxo groups.

In some embodiments, Ring E and Ring H is selected from those depicted in Table 1, below.

As defined above and described herein, each of Ring I and Ring J is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur

In some embodiments, each of Ring I and Ring J is independently a 6-membered aryl. In some embodiments, each of Ring I and Ring J is independently a 6-membered heteroaryl containing 1-4 heteroatoms independently selected from nitrogen, oxygen, and sulfur. In some embodiments, each of Ring I and Ring J is independently a 5 to 7-membered saturated or partially unsaturated carbocyclyl. In some embodiments, each of Ring I and Ring J is independently a 5 to 7-membered saturated or partially unsaturated heterocyclyl ring with 1-3 heteroatoms independently selected from boron, nitrogen, oxygen, silicon, and sulfur. In some embodiments, each of Ring I and Ring J is independently a 5-membered heteroaryl with 1-3 heteroatoms independently selected from nitrogen, oxygen and sulfur.

As defined above and described herein, Ring K is a fused ring selected from a 5-12 (e.g., 6-12) membered saturated or partially unsaturated carbocyclyl or heterocyclyl ring with 1-3 heteroatoms independently selected from boron, nitrogen, oxygen, silicon, and sulfur, wherein Ring H is optionally further substituted with 1-2 oxo groups.

In some embodiments, Ring K is a fused ring selected from a 5-12 (e.g., 6-12) membered saturated or partially unsaturated carbocyclyl. In some embodiments, Ring K is a 5-12 (e.g., 6-12) membered saturated or partially unsaturated heterocyclyl ring with 1-3 heteroatoms independently selected from boron, nitrogen, oxygen, silicon, and sulfur. In some embodiments, Ring K is a fused 5-6 membered saturated or partially unsaturated heterocyclyl ring with 1-3 heteroatoms independently selected from boron, nitrogen, oxygen, silicon, and sulfur. In some embodiments, Ring K is optionally further substituted with 1-2 oxo groups.

In some embodiments, Ring I, Ring J, and Ring K is selected from those depicted in Table 1, below.

As defined above and described herein, Ring M is selected from

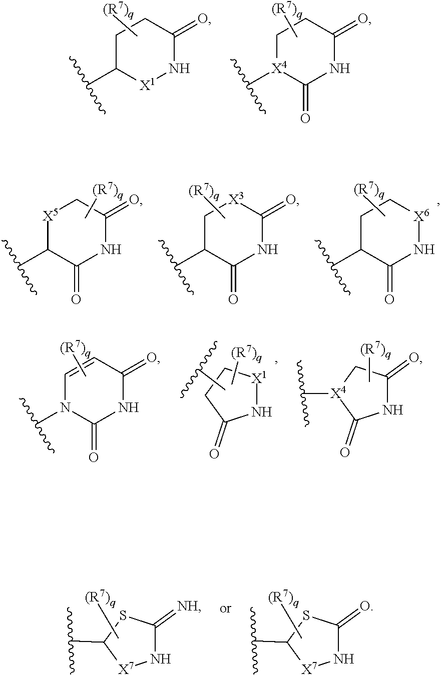

In some embodiments, Ring M is

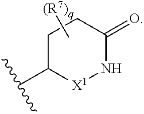

In some embodiments, Ring M is

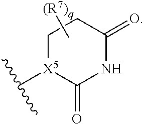

In some embodiments, Ring M is

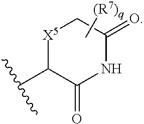

In some embodiments, Ring M is

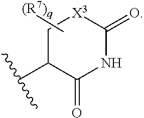

In some embodiments, Ring M is

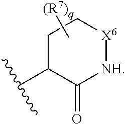

In some embodiments, Ring M is

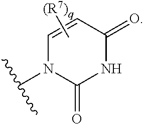

In some embodiments, Ring M is

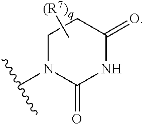

In some embodiments, Ring M is

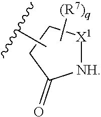

In some embodiments, Ring M is

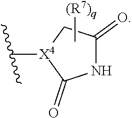

In some embodiments, Ring M is

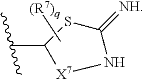

In some embodiments, Ring M is

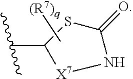

In some embodiments, Ring M is selected from those depicted in Table 1 below.

As defined above and described here, L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;

In some embodiments, L¹ is a covalent bond. In some embodiments, L¹ is a C₁₋₃ aliphatic. In some embodiments, L¹ is —CH₂—. In some embodiments, L¹ is —C(D)(H)—. In some embodiments, L¹ is —C(D)₂-. In some embodiments, L¹ is —CH₂CH₂—. In some embodiments, L¹ is —NR—. In some embodiments, L¹ is —NH—. In some embodiments, L¹ is —NMe-. In some embodiments, L¹ is —NEt-. In some embodiments, L¹ is —CH₂NR—. In some embodiments, L¹ is or —O—. In some embodiments, L¹ is —CH₂O—. In some embodiments, L¹ is —S—. In some embodiments, L¹ is —OC(O)—. In some embodiments, L¹ is —C(O)O—. In some embodiments, L¹ is —C(O)—. In some embodiments, L¹ is —S(O)—. In some embodiments, L¹ is —S(O)₂—. In some embodiments, L¹ is —NRS(O)₂—. In some embodiments, L¹ is —S(O)₂NR—. In some embodiments, L¹ is —NRC(O)—. In some embodiments, L¹ is —C(O)NR—.

In some embodiments, Ring L¹ is selected from those depicted in Table 1, below.

As defined above and described herein, is a single or double bond.

In some embodiments, [symbol] is a single bond. In some embodiments, [symbol] is a double bond.

In some embodiments, [symbol] is selected from those depicted in Table 1, below.

As defined above and described herein, m is 0, 1, 2, 3, 4, 5, 6, 7, 8, 9, 10, 11, 12, 13, 14, 15, or 16.

In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12. In some embodiments, m is 13. In some embodiments, m is 14. In some embodiments, m is 15. In some embodiments, m is 16.

In some embodiments, m is selected from those depicted in Table 1, below.

As defined above and described herein, n is 0, 1, 2, 3 or 4.

In some embodiments, n is 0. In some embodiments, n is 1. In some embodiments, n is 2. In some embodiments, n is 3. In some embodiments, n is 4.

In some embodiments, n is selected from those depicted in Table 1, below.

As defined above and described herein, p is 0 or 1.

In some embodiments, p is 0. In some embodiments, p is 1.

In some embodiments, p is selected from those depicted in Table 1, below.

As defined above and described herein, q is 0, 1, 2, 3 or 4.

In some embodiments, q is 0. In some embodiments, q is 1. In some embodiments, q is 2. In some embodiments, q is 3. In some embodiments, q is 4.

In some embodiments, q is selected from those depicted in Table 1 below.

In some embodiments, LBM is

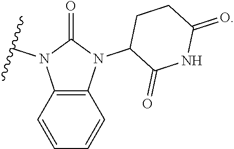 [SMILES: *n1c(=O)n(C2CCC(=O)NC2=O)c2ccccc21]

In some embodiments, LBM is

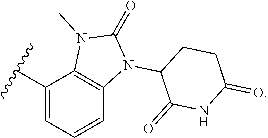 [SMILES: *c1cccc2c1n(C)c(=O)n2C1CCC(=O)NC1=O]

In some embodiments, LBM is

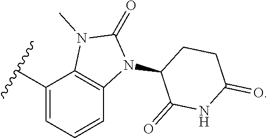 [SMILES: *c1cccc2c1n(C)c(=O)n2[C@H]1CCC(=O)NC1=O]

In some embodiments, LBM is

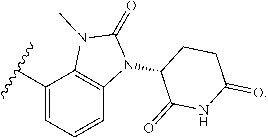 [SMILES: *c1cccc2c1n(C)c(=O)n2[C@@H]1CCC(=O)NC1=O]

In some embodiments, LBM is

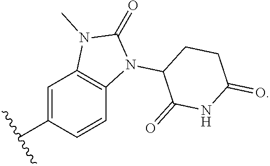 [SMILES: *c1ccc2c(c1)n(C)c(=O)n2C1CCC(=O)NC1=O]

In some embodiments, LBM is

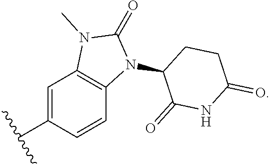 [SMILES: *c1ccc2c(c1)n(C)c(=O)n2[C@H]1CCC(=O)NC1=O]

In some embodiments, LBM is

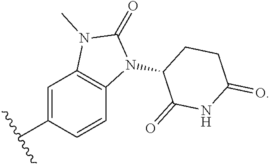 [SMILES: *c1ccc2c(c1)n(C)c(=O)n2[C@@H]1CCC(=O)NC1=O]

In some embodiments, LBM is

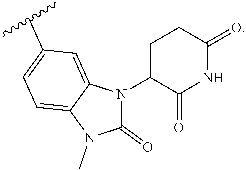 [SMILES: *c1ccc2c(c1)n(C1CCC(=O)NC1=O)c(=O)n2C]

In some embodiments, LBM is

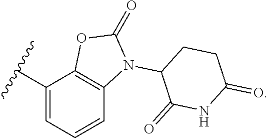 [SMILES: *c1cccc2c1oc(=O)n2C1CCC(=O)NC1=O]

In some embodiments, LBM is

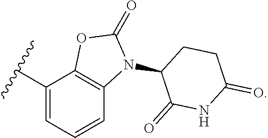 [SMILES: *c1cccc2c1oc(=O)n2[C@H]1CCC(=O)NC1=O]

In some embodiments, LBM is

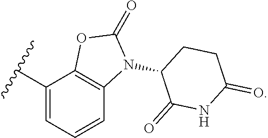 [SMILES: *c1cccc2c1oc(=O)n2[C@@H]1CCC(=O)NC1=O]

In some embodiments, LBM is

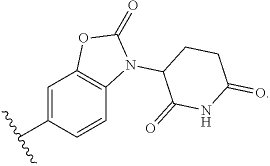 [SMILES: *c1ccc2c(c1)oc(=O)n2C1CCC(=O)NC1=O]

In some embodiments, LBM is

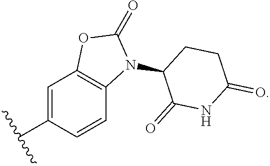 [SMILES: *c1ccc2c(c1)oc(=O)n2[C@H]1CCC(=O)NC1=O]

In some embodiments, LBM is

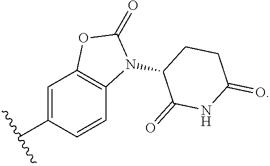 [SMILES: *c1ccc2c(c1)oc(=O)n2[C@@H]1CCC(=O)NC1=O]

In some embodiments, LBM is

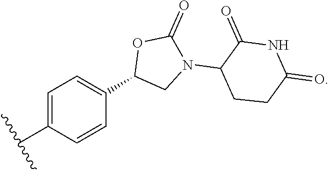 [SMILES: *c1ccc([C@H]2CN(C3CCC(=O)NC3=O)C(=O)O2)cc1]

In some embodiments, LBM is

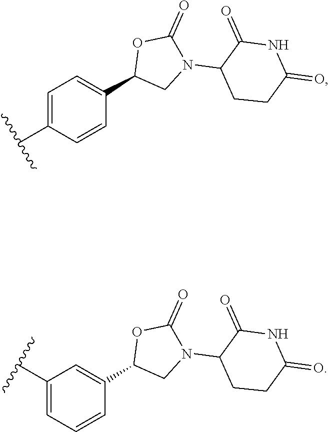 [SMILES: *c1ccc([C@@H]2CN(C3CCC(=O)NC3=O)C(=O)O2)cc1 ; *c1cccc([C@H]2CN(C3CCC(=O)NC3=O)C(=O)O2)c1]

In some embodiments, LBM is

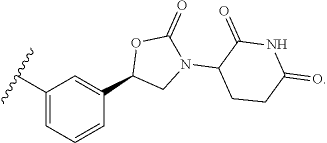 [SMILES: *c1cccc([C@@H]2CN(C3CCC(=O)NC3=O)C(=O)O2)c1]

In some embodiments, LBM is

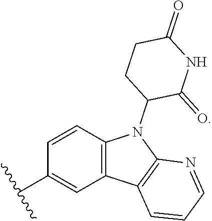 [SMILES: *c1ccc2c(c1)c1cccnc1n2C1CCC(=O)NC1=O]

In some embodiments, LBM is

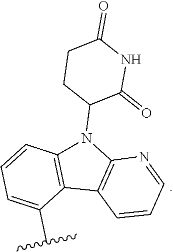 [SMILES: *c1cccc2c1c1cccnc1n2C1CCC(=O)NC1=O]

In some embodiments, LBM is

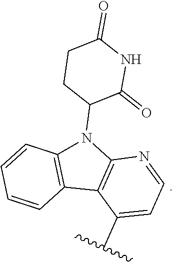 [SMILES: *c1ccnc2c1c1ccccc1n2C1CCC(=O)NC1=O]

In some embodiments, LBM is

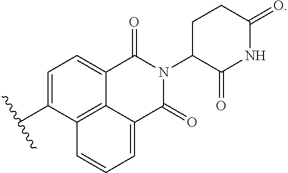 [SMILES: *c1ccc2c3c(cccc13)C(=O)N(C1CCC(=O)NC1=O)C2=O]

In some embodiments, LBM is

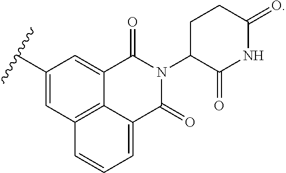 [SMILES: *c1cc2c3c(cccc3c1)C(=O)N(C1CCC(=O)NC1=O)C2=O]

In some embodiments, LBM is

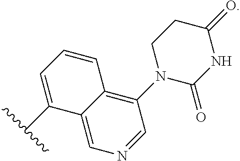 [SMILES: *c1cccc2c(N3CCC(=O)NC3=O)cncc12]

In some embodiments, LBM is

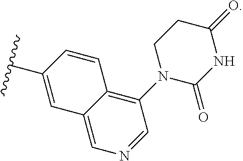 [SMILES: *c1ccc2c(N3CCC(=O)NC3=O)cncc2c1]

In some embodiments, LBM is

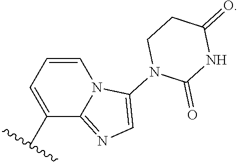 [SMILES: *c1cccn2c(N3CCC(=O)NC3=O)cnc12]

In some embodiments, LBM is

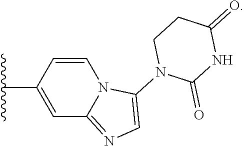 [SMILES: *c1ccn2c(N3CCC(=O)NC3=O)cnc2c1]

In certain embodiments, the present invention provides a compound of Formula I, wherein LBM is a MDM2 (i.e. human double minute 2 or HDM2) E3 ligase binding moiety thereby forming a compound of Formula I-i-1, I-i-2, I-i-3, I-i-4, I-i-5, I-i-6, I-i-7, I-i-8, I-i-9, I-i-10, I-i-11, I-i-12, I-i-13, I-i-14, I-i-15, I-i-16, I-i-17, or I-i-18 respectively:

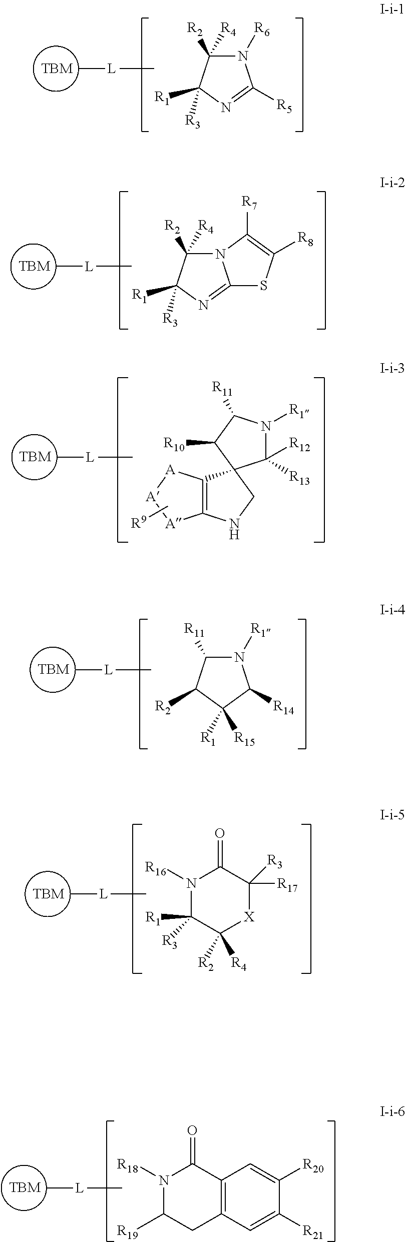

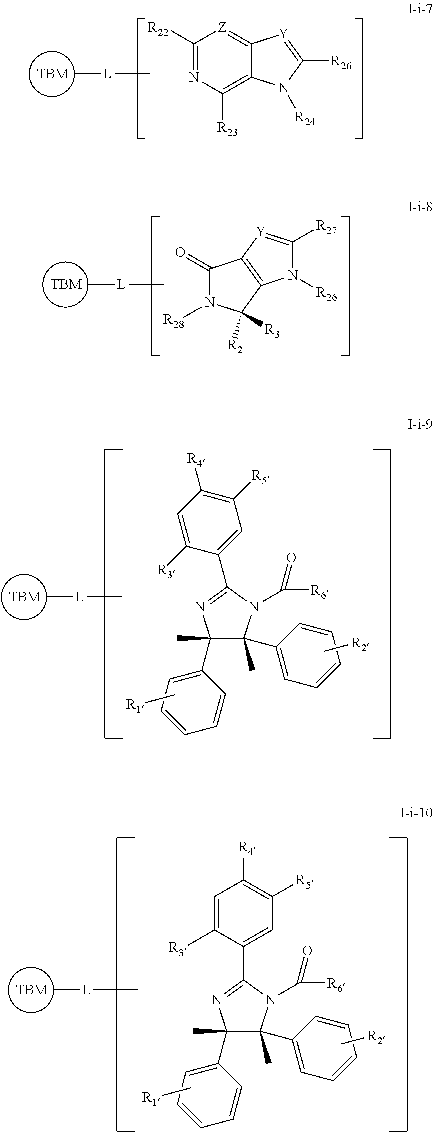

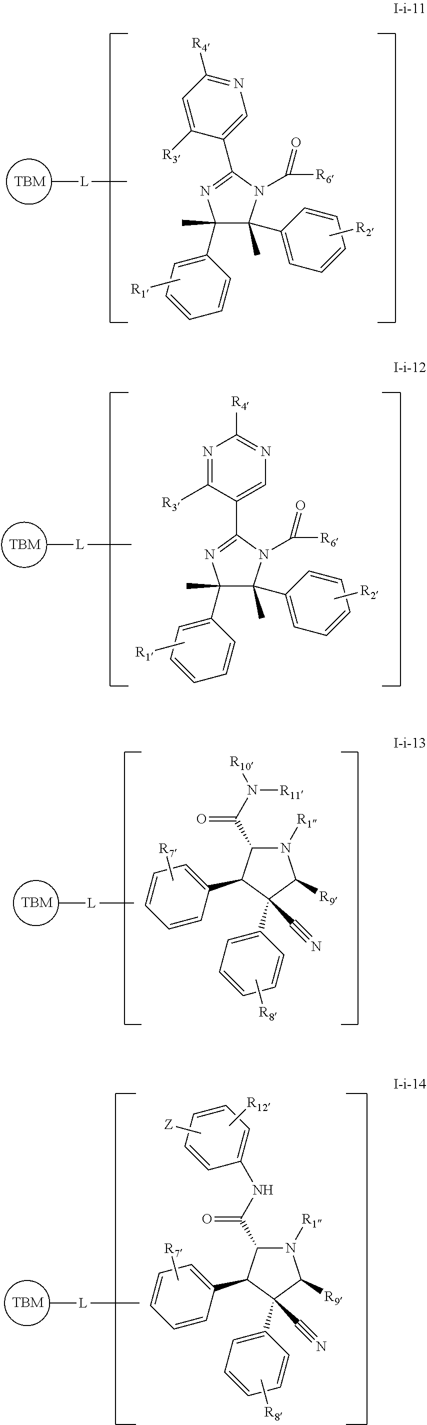

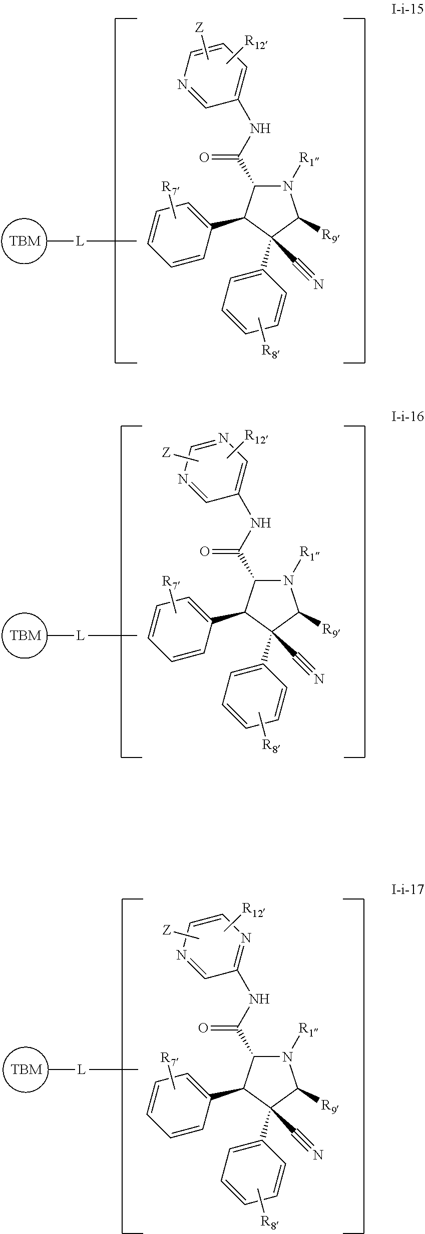

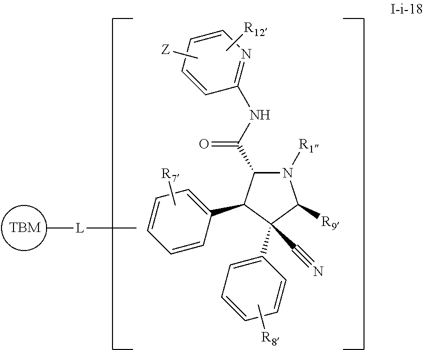

or a pharmaceutically acceptable salt thereof, wherein L and TBM are as defined above and described in embodiments herein, and wherein:
X is selected from —CR₂—, —O—, —S—, —S(O)—, —S(O)₂—, and —NR—;
each R is independently hydrogen or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are optionally taken together with their intervening atoms to form a 4-7 membered saturated, partially unsaturated, or heteroaryl ring having 0-3 heteroatoms, in addition to the atom from which they are attached, independently selected from nitrogen, oxygen, and sulfur.
Y and Z are independently selected from —CR═ and —N═;
Ring W is fused ring selected from benzo and a 5-6 membered heteroaryl with 1-4 heteroatoms independently selected from nitrogen, oxygen and sulfur;
R¹ and R² are independently an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R³ and R⁴ are independently selected from hydrogen and C₁₋₆ alkyl;
R⁵ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R⁶ is selected from hydrogen, —C(O)R, —C(O)OR, and —C(O)NR₂;
R⁷ is selected from hydrogen and R^{A};
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R⁸ is selected from —C(O)R and R^{A};
R⁹ is a mono-, bis-, or tri-substituent on Ring W, wherein each of the substituents are independently selected from halogen and an optionally substituted C₁₋₆ aliphatic;
R¹⁰ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R¹¹ is —C(O)OR or —C(O)NR₂;
R¹² and R¹³ are independently selected from hydrogen and R^{A}, or:
R¹² and R¹³ are optionally taken together with their intervening atoms to form an optionally substituted 3-8 membered saturated, partially unsaturated, carbocyclic or heterocyclic ring having 1-3 heteroatoms independently selected from nitrogen, oxygen, and sulfur;
R¹⁴ is R^{A};
R¹⁵ is —CN;
R¹⁶ is selected from R^{A}, —OR, —(CR₂)₀₋₆—C(O)R, —(CR₂)₀₋₆—C(O)OR, —(CR₂)₀₋₆—C(O)NR₂, —(CR₂)₀₋₆—S(O)₂R, —(CR₂)₀₋₆—N(R)S(O)₂R, —(CR₂)₀₋₆—S(O)₂NR₂;
R¹⁷ is selected from —(CR₂)₀₋₆—C(O)NR₂;
R¹⁸ and R¹⁹ are independently selected from hydrogen and R^{A};
R²⁰ and R²¹ are independently selected from hydrogen, R^{A}, halogen, and —OR, or:
R²⁰ and R²¹ are optionally taken together with their intervening atoms to form a fused 5-7 membered partially unsaturated carbocyclic or heterocyclic ring having 1-3 heteroatoms independently selected from nitrogen, oxygen, and sulfur, or a fused 5-6 membered heteroaryl ring having 1-3 heteroatoms independently selected from nitrogen, oxygen, and sulfur;
R²², R²³, R²⁵, and R²⁷ are independently selected from hydrogen, R^{A}, halogen, —C(O)R, —C(O)OR, —C(O)NR₂, —NR₂, —OR, —S(O)R, —S(O)₂R, —S(O)₂NR₂;
R²⁴, R²⁶, and R²⁸ are independently selected from hydrogen, R^{A}, —C(O)R, —C(O)OR, —C(O)NR₂, —S(O)R, —S(O)₂R, and —S(O)₂NR₂;
R^{1′} and R^{2′} are independently selected from halogen, —C≡CR, —CN, —CF₃, and —NO₂;
R^{3′} is —OR;
R^{4′}, R^{5′}, R^{6′} are independently selected from hydrogen, halogen, R^{A}, —CN, —CF₃, —NR₂, —OR, —SR, and —S(O)₂R;
R^{7′} is a mono-, bis-, or tri-substituent, wherein each of the substituents are independently selected from halogen;
R^{8′} is a mono-, bis-, or tri-substituent, wherein each of the substituents are independently selected from hydrogen, halogen, R^{A}, —CN, —C≡CR, —NO₂, and —OR;
R^{9′} is R^{A};
Z¹ is selected from hydrogen, halogen, and —OR;
R^{10′} and R^{11′} are independently selected from hydrogen and R^{A};
R^{12′} is selected from —C(O)R, —C(O)OR, —C(O)NR₂, —OR, —S(O)₂R, —S(O)₂NR₂, and —S(O)R; and
R^{1″} is selected from hydrogen and R^{A}.

In certain embodiments, the present invention provides a compound of Formula I, wherein LBM is a MDM2 (i.e. human double minute 2 or HDM2) E3 ligase binding moiety thereby forming a compound of Formula I-i-19, I-i-20, or I-i-21 respectively:

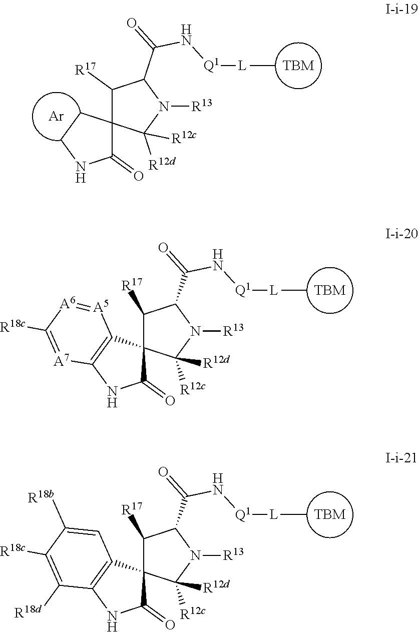

or a pharmaceutically acceptable salt thereof, wherein L and TBM are as defined above and described in embodiments herein, and wherein:
R^{1″} is selected from hydrogen and R^{A};
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R¹⁰ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R₁₂ and R₁₃ are each independently selected from hydrogen and R^{A}, or:
R¹² and R¹³ are optionally taken together with their intervening atoms to form an optionally substituted 4-8 membered saturated, partially unsaturated, carbocyclic or heterocyclic ring having 1-3 heteroatoms independently selected from nitrogen, oxygen, and sulfur;
A⁵ is selected from —C(R^{18a})═ and —N═;
A⁶ is selected from —C(R^{18b})═ and —N═;
A⁷ is selected from —C(R^{18d})═ and —N═;
R^{18a}, R^{18b}, R^{18c}, and R^{18d} are each independently selected from hydrogen, halogen, R^{A}, and —OR;
each R is independently hydrogen or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring W is an optionally substituted fused ring selected from benzo and a 5-6 membered heteroaryl with 1-4 heteroatoms independently selected from nitrogen, oxygen and sulfur; and
Q¹ is and optionally substituted bivalent group selected from alkylenyl, phenylenyl, heteroarylenyl, cycloalkylenyl, and heterocyclenyl.

In certain embodiments, the present invention provides a compound of Formula I, wherein LBM is an IAP E3 ubiquitin ligase binding moiety thereby forming a compound of Formula I-j-1, I-j-2, I-j-3, or I-j-4 respectively:

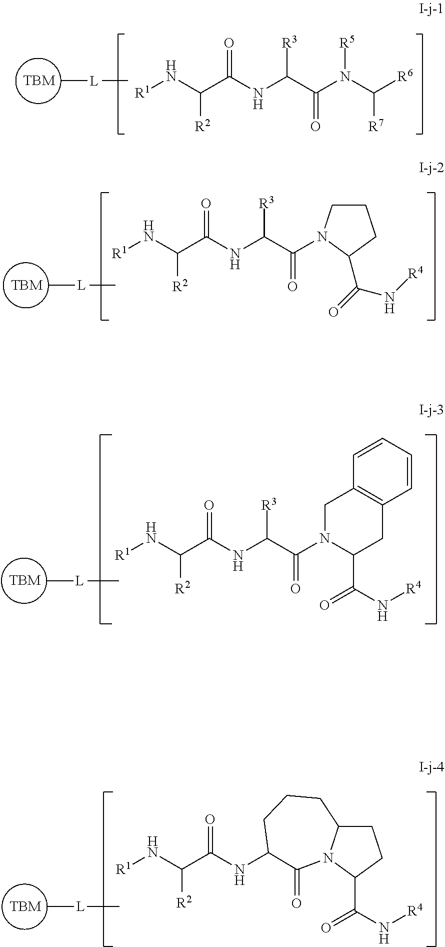

or a pharmaceutically acceptable salt thereof, wherein L and TBM are as defined above and described in embodiments herein, and wherein each of the variables R¹, R², R³, R⁴, R⁵, R⁶, and R⁷, is as defined and described in WO 2017/011590 and US 2017/0037004, the entirety of each of which is herein incorporated by reference.

In certain embodiments, the present invention provides a compound of Formula I, wherein LBM is an IAP binding moiety thereby forming a compound of Formula I-k-1:

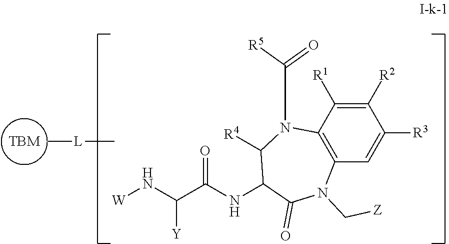

or a pharmaceutically acceptable salt thereof, wherein L and TBM are as defined above and described in embodiments herein, and wherein each of the variables W, Y, Z, R¹, R², R³, R⁴, and R⁵ is as described and defined in WO 2014/044622, US 2015/0225449. WO 2015/071393, and US 2016/0272596, the entirety of each of which is herein incorporated by reference.

In certain embodiments, the present invention provides a compound of Formula I-DB:

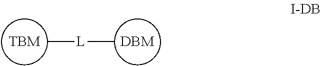

or a pharmaceutically acceptable salt thereof, wherein:
TBM is target binding moiety capable of binding to a targeted protein(s).
L is a bivalent moiety that connects TBM to DBM; and
DBM is a DCAF1 binding moiety capable of binding to DCAF1 protein.

In certain embodiments, the present invention provides a compound of Formula I-DB, wherein DBM is a DCAF1 binding moiety of Formula I-k-2-a:

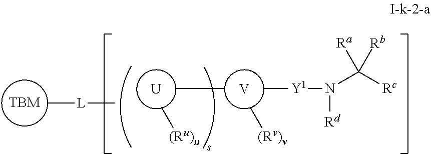

or a pharmaceutically acceptable salt thereof, wherein L and TBM are as defined and described herein, and wherein:
Ring T is phenyl, a 5-7 membered saturated or partially unsaturated carbocyclyl or heterocyclyl with 1-3 heteroatoms independently selected from nitrogen, oxygen, and sulfur, or a 5-6 membered heteroaryl with 1-4 heteroatoms independently selected from nitrogen, oxygen and sulfur;
Ring U is phenyl, a 4-7 membered partially unsaturated carbocyclyl or heterocyclyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur;
Ring V is phenylenyl, a 4-10 membered partially unsaturated carbocyclylenyl or heterocyclylenyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur;
Y¹ is a C₁₋₃ hydrocarbon chain wherein each methylene is optionally substituted with —CR₂—, —CR(OR)—, —C(O)—, —C(NR)—, —C(NOR)—, —S(O)—, or —S(O)₂—;
R^{a} is an optionally substituted C₁₋₆ aliphatic or

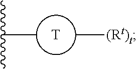

R^{b} is hydrogen, an optionally substituted C₁₋₆ aliphatic, phenyl, or a 5-6 membered heteroaryl with 1-4 heteroatoms independently selected from nitrogen, oxygen and sulfur, or:
   R^{a} and R^{b} are optionally taken together with their intervening atoms to form an optionally substituted 9-10 membered saturated or partially unsaturated bicyclic ring having 0-3 heteroatoms independently selected from nitrogen, oxygen, and sulfur, or:
   when Y is —C(NR)—, R^{b} is optionally taken together with R of —C(NR)— with their intervening atoms to form a 5-7 membered partially unsaturated heterocyclyl with 0-1 heteroatoms, in addition to the 2 nitrogen atoms within the heterocyclyl, independently selected from nitrogen, oxygen, and sulfur;
R^{c} is —CR₂CONR₂, a 5-7 membered saturated or partially unsaturated carbocyclyl or heterocyclyl with 1-3 heteroatoms independently selected from nitrogen, oxygen, and sulfur, or a 5-6 membered heteroaryl with 1-4 heteroatoms independently selected from nitrogen, oxygen and sulfur;
R^{d} is hydrogen, or:
   when R^{c} is —CR₂CONR₂, R^{d} is optionally taken together with a single R of —CR₂CONR₂ with their intervening atoms to form a 5-7 membered saturated or partially unsaturated heterocyclyl with 0-3 heteroatoms, in addition to the nitrogen atom to which R^{d} is attached, independently selected from nitrogen, oxygen, and sulfur;
R^{t}, R^{u}, and R^{v} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, —P(O)R₂, —P(O)(OR)₂, —P(O)(OR)NR₂, and —P(O)(NR₂)₂;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-7 membered saturated or partially unsaturated ring having 0-3 heteroatoms, in addition to the atom to which they are attached, independently selected from nitrogen, oxygen, and sulfur;
s is 0 or 1; and
each of t, u, and v are independently 0, 1, 2, 3, or 4;
wherein DBM is further optionally substituted with

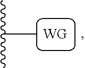

wherein

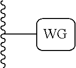

is a warhead group.

In certain embodiments, the present invention provides a compound of Formula I-DB, wherein DBM is a DCAF1 binding moiety of Formula I-k-2-b:

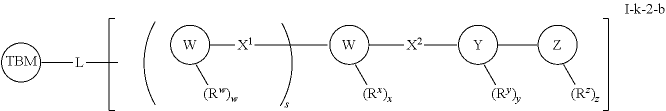

or a pharmaceutically acceptable salt thereof, wherein L and TBM are as defined and described herein, and wherein:
Ring W is a 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
Ring X is phenylenyl, a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur;
Ring Y is a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
Ring Z is phenyl, naphthyl, a 9-10 membered saturated or partially unsaturated bicyclic heterocyclylenyl with 1-3 heteroatoms independently selected from nitrogen, oxygen, and sulfur, or a 5-13 membered monocyclic, bicyclic, or tricyclic heteroarylenyl with 1-5 heteroatoms independently selected from nitrogen, oxygen and sulfur;
R^{w}, R^{x}, R^{y}, and R^{z} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, —P(O)R₂, —P(O)(OR)₂, —P(O)(OR)NR₂, and —P(O)(NR₂)₂, or:
   an R^{x} group on Ring X and an R^{y} group or Ring Y are optionally taken together with their intervening atoms to form a 5-8 membered saturated or partially unsaturated ring having 0-3 heteroatoms independently selected from nitrogen, oxygen, and sulfur;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-7 membered saturated or partially unsaturated ring having 0-3 heteroatoms, in addition to the atom to which they are attached, independently selected from nitrogen, oxygen, and sulfur;
each of X¹ and X² is independently a covalent bond, spiro-fusion between Ring X and Ring Y, —CR₂—, —CR(OR)—, —CRF—, —CF₂—, —NR—, —O—, —S—, or —S(O)₂—;
s is 0 or 1; and
each of w, x, y, and z are independently 0, 1, 2, 3, or 4;
wherein DBM is further optionally substituted with

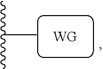

wherein

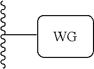

is a warhead group.

As described above and defined herein, Ring T is phenyl, a 5-7 membered saturated or partially unsaturated carbocyclyl or heterocyclyl with 1-3 heteroatoms independently selected from nitrogen, oxygen, and sulfur, or a 5-6 membered heteroaryl with 1-4 heteroatoms independently selected from nitrogen, oxygen and sulfur.

In some embodiments, Ring T is phenyl. In some embodiments, Ring T is a 5-7 membered saturated or partially unsaturated carbocyclyl. In some embodiments, Ring T is a 5-7 membered saturated or partially unsaturated heterocyclyl with 1-3 heteroatoms independently selected from nitrogen, oxygen, and sulfur. In some embodiments, Ring T is a 5-6 membered heteroaryl with 1-4 heteroatoms independently selected from nitrogen, oxygen and sulfur.

In some embodiments, Ring T is cyclohexyl, cyclohexenyl, isothiazolyl, phenyl, or pyridyl.

In some embodiments, Ring T is as depicted in the compounds of Table 1, below.

As described above and defined herein, Ring U is phenyl, a 4-7 membered partially unsaturated carbocyclyl or heterocyclyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur.

In some embodiments, Ring U is phenyl. In some embodiments, Ring U is a 4-7 membered partially unsaturated carbocyclyl. In some embodiments, Ring U is a 4-7 membered partially unsaturated heterocyclyl with 1-3 heteroatoms independently selected from nitrogen, oxygen, and sulfur. In some embodiments, Ring U is a 5-9 membered monocyclic or bicyclic heteroarylenyl with 1-4 heteroatoms independently selected from nitrogen, oxygen and sulfur.

In some embodiments, Ring U is cyclobutyl, azetinyl, cyclohexyl, cyclohexenyl, tetrahydro-2H-pyranyl, pyrrolidinyl, 4,5-dihydro-1H-pyrazolyl, piperidinyl, phenyl, isoxazolyl, isothiazolyl, pyrazolyl, pyridyl, pyridazinyl, pyrimidinyl, indolyl, benzoimidazolyl, pyrazolo[1,5-a]pyridyl, or [1,2,4]triazolo[1,5-a]pyridyl.

In some embodiments, Ring U is as depicted in the compounds of Table 1, below.

As described above and defined herein, Ring V is phenylenyl, a 4-10 membered partially unsaturated carbocyclylenyl or heterocyclylenyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur.

In some embodiments, Ring V is phenylenyl. In some embodiments, Ring V is a 4-10 membered partially unsaturated carbocyclylenyl or heterocyclylenyl with 1-3 heteroatoms independently selected from nitrogen, oxygen, and sulfur. In some embodiments, Ring V is a 5-9 membered monocyclic or bicyclic heteroarylenyl with 1-4 heteroatoms independently selected from nitrogen, oxygen and sulfur.

In some embodiments, Ring V is cyclobutylenyl, azetinylenyl, cyclopentylenyl cyclohexyl, phenylenyl, pyrrolylenyl, imidazolylenyl, pyrazolylenyl, 1,2,3-triazolylenyl, 1,2,4-triazolylenyl, pyridylenyl, indazolyl, 1,2,3,6-tetrahydropyridinyl, 4,5,6,7-tetrahydro-1H-pyrazolo[4,3-b]pyridyl, benzoimidazolyl, 3,4-dihydroquinolinyl, or 4,5,6,7-tetrahydro-1H-pyrazolo[4,3-c]pyridyl.

In some embodiments, Ring V is as depicted in the compounds of Table 1, below.

As described above and defined herein, Ring W is a 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur.

In some embodiments, Ring W is a 3-11 membered saturated or partially unsaturated monocyclic, bicyclic, bridged bicyclic, or spirocyclic carbocyclyl. In some embodiments, Ring W is a 3-11 membered saturated or partially unsaturated monocyclic, bicyclic, bridged bicyclic, or spirocyclic heterocyclyl with 1-3 heteroatoms independently selected from nitrogen, oxygen, and sulfur.

In some embodiments, Ring W is cyclopropyl, cyclobutyl, azetinyl, pyrrolidinyl, cyclohexyl, piperidinyl, piperazinyl, 3,6-dihydro-2H-pyranyl, tetrahydro-2H-pyranyl, morpholinyl, piperzinyl, 2,7-diazaspiro[3.5]nonanyl, 3,4-dihydro-2H-pyrido[3,2-b][1,4]oxazinyl, 2-oxa-5-azabicyclo[2.2.1]heptanyl, 6-oxa-3-azabicyclo[3.1.1]heptanyl, or 2-oxa-5-azabicyclo[2.2.2]octanyl.

In some embodiments, Ring W is as depicted in the compounds of Table 1, below.

As described above and defined herein, Ring X is phenylenyl, a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur.

In some embodiments, Ring X is phenylenyl. In some embodiments, Ring X is a 3-11 membered saturated or partially unsaturated monocyclic, bicyclic, bridged bicyclic, or spirocyclic carbocyclylenyl. In some embodiments, Ring X is a 3-11 membered saturated or partially unsaturated monocyclic, bicyclic, bridged bicyclic, or spirocyclic heterocyclylenyl with 1-3 heteroatoms independently selected from nitrogen, oxygen, and sulfur. In some embodiments, Ring X is a 5-9 membered monocyclic or bicyclic heteroarylenyl with 1-4 heteroatoms independently selected from nitrogen, oxygen and sulfur.

In some embodiments, Ring X is phenylenyl, imidazolylenyl, pyrazolylenyl, oxazolylenyl, thiazolylenyl, 1,2-thiazinanylenyl, pyridylenyl, pyridazinylenyl, pyrimidinylenyl, 2,6-diazaspiro[3.5]nonanylenyl, 2,3-dihydro-1H-pyrrolo[2,3-b]pyridylenyl, 2,3-dihydro-1H-pyrrolo[3,2-c]pyridylenyl, 1H-pyrrolo[2,3-b]pyridylenyl, 3H-imidazo[4,5-b]pyridylenyl, 9H-purinylenyl, 1,2,3,4-tetrahydro-1,8-naphthyridinylenyl, or 1,2,3,4-tetrahydro-1,6-naphthyridinylenyl.

In some embodiments, Ring X is as depicted in the compounds of Table 1, below.

As described above and defined herein, Ring Y is a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur.

In some embodiments, Ring Y is a 3-11 membered saturated or partially unsaturated monocyclic, bicyclic, bridged bicyclic, or spirocyclic carbocyclyl. In some embodiments, Ring Y is a 3-11 membered saturated or partially unsaturated monocyclic, bicyclic, bridged bicyclic, or spirocyclic heterocyclyl with 1-3 heteroatoms independently selected from nitrogen, oxygen, and sulfur.

In some embodiments, Ring Y is cyclohexylenyl, azetidinylenyl, pyrrolidinylenyl, imidazolylenyl, piperidinylenyl, piperzinylenyl, azepanylenyl, 8-azabicyclo[3.2.1]octanylenyl, 2-azabicyclo[3.2.1]octanylenyl, 2-azabicyclo[3.2.2]nonanylenyl, octahydro-1H-pyrrolo[3,2-b]pyridylenyl, decahydro-1,5-naphthyridinylenyl, 9-azabicyclo[3.3.1]nonanylenyl, 5-azaspiro[3.5]nonanylenyl, 2-oxa-5-azaspiro[3.5]nonanylenyl, or 2,6-diazaspiro[3.5]nonanylenyl.

In some embodiments, Ring Y is as depicted in the compounds of Table 1, below.

As described above and defined herein, Ring Z is phenyl, naphthyl, a 9-10 membered saturated or partially unsaturated bicyclic heterocyclylenyl with 1-3 heteroatoms independently selected from nitrogen, oxygen, and sulfur, or a 5-13 membered monocyclic, bicyclic, or tricyclic heteroarylenyl with 1-5 heteroatoms independently selected from nitrogen, oxygen and sulfur.

In some embodiments, Ring Z is phenyl. In some embodiments, Ring Z is naphthyl. In some embodiments, Ring Z is a 9-10 membered saturated or partially unsaturated bicyclic heterocyclylenyl with 1-3 heteroatoms independently selected from nitrogen, oxygen, and sulfur. In some embodiments, Ring Z is a 5-13 membered monocyclic, bicyclic, or tricyclic heteroarylenyl with 1-5 heteroatoms independently selected from nitrogen, oxygen and sulfur.

In some embodiments, Ring Z is 1,2,3-triazolyl, thiazolyl, pyrazolyl, phenyl, pyridyl, pyridazinyl, pyrimidinyl, indazolyl, benzo[d]isoxazolyl, benzo[d]isothiazolyl, pyrazolo[1,5-a]pyrimidinyl, 2,3-dihydro-1H-pyrrolo[2,3-c]pyridinyl, 6,7-dihydro-5H-cyclopenta[b]pyridinyl, 2,3-dihydro-1H-pyrrolo[3,2-c]pyridinyl, naphthyl, quinolinyl, isoquinolinyl, 1,6-naphthyridinyl, phthalazinyl, quinazolinyl, 2,7-naphthyridinyl, or tetrazolo[1,5-a]quinoxalinyl.

In some embodiments, Ring Z is as depicted in the compounds of Table 1, below.

As described above and defined herein, R^{a} is an optionally substituted C₁₋₆ aliphatic or

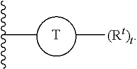

In some embodiments, R^{a} is an optionally substituted C₁₋₆ aliphatic. In some embodiments, R^{a} is

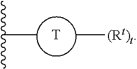

In some embodiments, Ring R^{a} is methyl.

In some embodiments, Ring R^{a} is as depicted in the compounds of Table 1, below.

As described above and defined herein, R^{b} is hydrogen, an optionally substituted C₁₋₆ aliphatic, phenyl, or a 5-6 membered heteroaryl with 1-4 heteroatoms independently selected from nitrogen, oxygen and sulfur, or R^{a} and R^{b} are optionally taken together with their intervening atoms to form an optionally substituted 9-10 membered saturated or partially unsaturated bicyclic ring having 0-3 heteroatoms independently selected from nitrogen, oxygen, and sulfur, or when Y is —C(NR)—, R^{b} is optionally taken together with R of —C(NR)— with their intervening atoms to form a 5-7 membered partially unsaturated heterocyclyl with 0-1 heteroatoms, in addition to the 2 nitrogen atoms within the heterocyclyl, independently selected from nitrogen, oxygen, and sulfur.

In some embodiments, R^{b} is hydrogen. In some embodiments, R^{b} is hydrogen is an optionally substituted C₁₋₆ aliphatic. In some embodiments, R^{b} is hydrogen is phenyl. In some embodiments, R^{b} is hydrogen is a 5-6 membered heteroaryl with 1-4 heteroatoms independently selected from nitrogen, oxygen and sulfur. In some embodiments, R^{a} and R^{b} are optionally taken together with their intervening atoms to form an optionally substituted 9-10 membered saturated or partially unsaturated bicyclic ring having 0-3 heteroatoms independently selected from nitrogen, oxygen, and sulfur. In some embodiments, when Y is —C(NR)—, R^{b} is optionally taken together with R of —C(NR)— with their intervening atoms to form a 5-7 membered partially unsaturated heterocyclyl with 0-1 heteroatoms, in addition to the 2 nitrogen atoms within the heterocyclyl, independently selected from nitrogen, oxygen, and sulfur.

In some embodiment, R^{b} is methyl, cyclopropyl, phenyl, —CO₂H, —CH₂cyclopropyl, —CH₂OH, —CH₂OMe, or —CH₂CO₂H.

In some embodiments, Ring R^{b} is as depicted in the compounds of Table 1, below.

As described above and defined herein, R^{c} is —CR₂CONR₂, a 5-7 membered saturated or partially unsaturated carbocyclyl or heterocyclyl with 1-3 heteroatoms independently selected from nitrogen, oxygen, and sulfur, or a 5-6 membered heteroaryl with 1-4 heteroatoms independently selected from nitrogen, oxygen and sulfur.

In some embodiments, R^{c} is —CR₂CONR₂. In some embodiments, R^{c} is a 5-7 membered saturated or partially unsaturated carbocyclyl. In some embodiments, R^{c} is a 5-7 membered saturated or partially unsaturated heterocyclyl with 1-3 heteroatoms independently selected from nitrogen, oxygen, and sulfur. In some embodiments, R^{c} is a 5-6 membered heteroaryl with 1-4 heteroatoms independently selected from nitrogen, oxygen and sulfur.

In some embodiments, R^{c} is —CH₂CONH₂, —CH(Me)CONH₂, —CH₂CONHMe, —CH₂CONHEt, —CH₂CONHCH₂Ph, —CH₂CONHcyclopropyl, pyrrolidin-2-onyl, piperidin-2-only, or isoxazolyl.

In some embodiments, Ring R^{c} is as depicted in the compounds of Table 1, below.

As described above and defined herein, R^{d} is hydrogen, or when R^{c} is —CR₂CONR₂, R^{d} is optionally taken together with a single R of —CR₂CONR₂ with their intervening atoms to form a 5-7 membered saturated or partially unsaturated heterocyclyl with 0-3 heteroatoms, in addition to the nitrogen atom to which R^{d} is attached, independently selected from nitrogen, oxygen, and sulfur.

In some embodiments, R^{d} is hydrogen.

In some embodiments, Ring R^{d} is as depicted in the compounds of Table 1, below.

As described above and defined herein, R^{t}, R^{u}, R^{v}, R^{w}, R^{x}, R^{y}, and R^{z} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, —P(O)R₂, —P(O)(OR)₂, —P(O)(OR)NR₂, and —P(O)(NR₂)₂, or an R^{x} group on Ring X and an R^{y} group or Ring Y are optionally taken together with their intervening atoms to form a 5-8 membered saturated or partially unsaturated ring having 0-3 heteroatoms independently selected from nitrogen, oxygen, and sulfur.

In some embodiments, one or more of R^{t}, R^{u}, R^{v}, R^{w}, R^{x}, R^{y}, and R^{z} is hydrogen. In some embodiments, one or more of R^{t}, R^{u}, R^{v}, R^{w}, R^{v}, R^{y}, and R^{z} is oxo. In some embodiments, one or more of R^{t}, R^{u}, R^{v}, R^{w}, R^{x}, R^{y}, and R^{z} is R^{A}. In some embodiments, one or more of R^{t}, R^{u}, R^{v}, R^{w}, R^{x}, R^{y}, and R^{z} is halogen. In some embodiments, one or more of R^{t}, R^{u}, R^{v}, R^{w}, R^{x}, R^{y}, and R^{z} is —CN. In some embodiments, one or more of R^{t}, R^{u}, R^{v}, R^{w}, R^{x}, R^{y}, and R^{z} is —NO₂. In some embodiments, one or more of R^{t}, R^{u}, R^{v}, R^{w}, R^{x}, R^{y}, and R^{z} is —OR. In some embodiments, one or more of R^{t}, R^{u}, R^{v}, R^{w}, R^{x}, R^{y}, and R^{z} is —SR. In some embodiments, one or more of R^{t}, R^{u}, R^{v}, R^{w}, R^{x}, R^{y}, and R^{z} is —NR₂. In some embodiments, one or more of R^{t}, R^{u}, R^{v}, R^{w}, R^{x}, R^{y}, and R^{z} is —SiR₃. In some embodiments, one or more of R^{t}, R^{u}, R^{v}, R^{w}, R^{x}, R^{y}, and R^{z} is —S(O)₂R. In some embodiments, one or more of R^{t}, R^{u}, R^{v}, R^{w}, R^{x}, R^{y}, and R^{z} is —S(O)₂NR₂. In some embodiments, one or more of R^{t}, R^{u}, R^{v}, R^{w}, R^{x}, R^{y}, and R^{z} is —S(O)R. In some embodiments, one or more of R^{t}, R^{u}, R^{v}, R^{w}, R^{x}, R^{y}, and R^{z} is —C(O)R. In some embodiments, one or more of R^{t}, R^{u}, R^{v}, R^{w}, R^{x}, R^{y}, and R^{z} is —C(O)OR. In some embodiments, one or more of R^{t}, R^{u}, R^{v}, R^{w}, R^{x}, R^{y}, and R^{z} is —C(O)NR₂. In some embodiments, one or more of R^{t}, R^{u}, R^{v}, R^{w}, R^{x}, R^{y}, and R^{z} is —C(O)NROR. In some embodiments, one or more of R^{t}, R^{u}, R^{v}, R^{w}, R^{x}, R^{y}, and R^{z} is —OC(O)R. In some embodiments, one or more of R^{t}, R^{u}, R^{v}, R^{w}, R^{x}, R^{y}, and R^{z} is —OC(O)NR₂. In some embodiments, one or more of R^{t}, R^{u}, R^{v}, R^{w}, R^{x}, R^{y}, and R^{z} is —OP(O)R₂. In some embodiments, one or more of R^{t}, R^{u}, R^{v}, R^{w}, R^{x}, R^{y}, and R^{z} is —OP(O)(OR)₂. In some embodiments, one or more of R^{t}, R^{u}, R^{v}, R^{w}, R^{x}, R^{y}, and R^{z} is —OP(O)(OR)NR₂. In some embodiments, one or more of R^{t}, R^{u}, R^{v}, R^{w}, R^{x}, R^{y}, and R^{z} is —OP(O)(NR₂)₂. In some embodiments, one or more of R^{t}, R^{u}, R^{v}, R^{w}, R^{x}, R^{y}, and R^{z} is —NRC(O)OR. In some embodiments, one or more of R^{t}, R^{u}, R^{v}, R^{w}, R^{x}, R^{y}, and R^{z} is —NRC(O)R. In some embodiments, one or more of R^{t}, R^{u}, R^{v}, R^{w}, R^{x}, R^{y}, and R^{z} is —NRC(O)N(R)₂. In some embodiments, one or more of R^{t}, R^{u}, R^{v}, R^{w}, R^{x}, R^{y}, and R^{z} is —NRS(O)₂R. In some embodiments, one or more of R^{t}, R^{u}, R^{v}, R^{w}, R^{x}, R^{y}, and R^{z} is —NP(O)R₂. In some embodiments, one or more of R^{t}, R^{u}, R^{v}, R^{w}, R^{x}, R^{y}, and R^{z} is —NRP(O)(OR)₂. In some embodiments, one or more of R^{t}, R^{u}, R^{v}, R^{w}, R^{x}, R^{y}, and R^{z} is —NRP(O)(OR)NR₂. In some embodiments, one or more of R^{t}, R^{u}, R^{v}, R^{w}, R^{x}, R^{y}, and R^{z} is —NRP(O)(NR₂)₂. In some embodiments, one or more of R^{t}, R^{u}, R^{v}, R^{w}, R^{x}, R^{y}, and R^{z} is —P(O)R₂. In some embodiments, one or more of R^{t}, R^{u}, R^{v}, R^{w}, R^{x}, R^{y}, and R^{z} is —P(O)(OR)₂. In some embodiments, one or more of R^{t}, R^{u}, R^{v}, R^{w}, R^{x}, R^{y}, and R^{z} is —P(O)(OR)NR₂. In some embodiments, one or more of R^{t}, R^{u}, R^{v}, R^{w}, R^{x}, R^{y}, and R^{z} is —P(O)(NR₂)₂. In some embodiments, an R^{x} group on Ring X and an R^{y} group or Ring Y are taken together with their intervening atoms to form a 5-8 membered saturated or partially unsaturated ring having 0-3 heteroatoms independently selected from nitrogen, oxygen, and sulfur.

In some embodiments, R^{t} is hydrogen, oxo, fluoro, chloro, —CN, methyl, —CONH₂, —OH, or —OMe.

In some embodiments, R^{u} is hydrogen, oxo, fluoro, chloro, —CN, methyl, —CO₂H, —CO₂Me, —CONH₂, —C(O)CHCH₂, —OH, —OMe, —CH₂CHF₂, —CH₂OMe, —CH₂CO₂H, —CH₂SO₂Me, —CH₂CH₂O₂H, —CH₂CH₂SO₂Me, —CH₂CH₂OMe, —NHC(O)CHCH₂, tetrazolyl, or N-methyltetrazolyl.

In some embodiments, R^{v} is hydrogen, oxo, methyl, isopropyl, —CH₂cyclopropyl, —CH₂cyclopentyl, —CH₂cyclohexyl, —CH₂morpholinyl, —CH₂Ph, —CH₂thiazolyl, —CH₂pyrimidinyl, —CH₂CH₂OMe, —CH₂CH₂Ph, —C(O)Me, —C(O)CHCH₂, —C(O)Ph, —C(O)pyrimidinyl, —NH₂, —NHC(O)CHCH₂, —CH₂NHC(O)CHCH₂, —CCNHC(O)CHCH₂, —NHcyclohexyl, —NHphenyl, or —NHpyrimidinyl,

In some embodiments, R^{w} is hydrogen, oxo, fluoro, methyl, ethyl, n-propyl, b-butyl, —CH₂CH₂OMe, —C(O)CHCH₂, —NHC(O)CHCH₂, —N(Me)C(O)CHCH₂, —CH₂NHC(O)CHCH₂, or

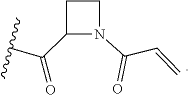 [SMILES: *C(=O)C1CCN1C(=O)C=C]

In some embodiments, R^{x} is hydrogen, oxo, fluoro, chloro, methyl, —CF₃, —CH₂OH, —CN, —OH, —OMe, —NH₂, or —N(Me)CH₂CH₂CH₂N(Me)C(O)CHCH₂.

In some embodiments, R^{y} is hydrogen, oxo, fluoro, methyl, —CH₂F, —CH₂OH, —CO₂H, —C(O)NH₂, —OH, —OMe, or —S(O)₂NH₂.

In some embodiments, R^{x} and R^{y}, are taken together by —CH₂CH₂— or —CH₂CH₂CH₂—.

In some embodiments, R^{z} is hydrogen, oxo, fluoro, chloro, —CN, methyl, isobutyl, —CF₃, —CH₂CF₃, —CH₂OH, —CH₂CO₂Me, —CH(OH)Me, —CH(NH₂)cyclopropyl, —CH₂Ph, —OH, —OMe, —OCF₃, —OiPr, OPh, —NHC(O)Me, —NHC(O)CHCH₂, —S(O)₂NH₂, 1,2,3-triazolyl, piperdinyl, N-methylpiperdinyl, phenyl, or pyridyl.

In some embodiments, R^{t}, R^{u}, R^{v}, R^{w}, R^{x}, R^{y}, and R^{z} are as depicted in the compounds of Table 1, below.

As described above and defined herein, each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{A} is an optionally substituted C₁₋₆ aliphatic. In some embodiments, R^{A} is an optionally substituted phenyl. In some embodiments, R^{A} is an optionally substituted 3-7 membered saturated or partially unsaturated carbocyclic. In some embodiments, R^{A} is an optionally substituted saturated or partially unsaturated heterocyclic ring having 1-2 heteroatoms independently selected from nitrogen, oxygen, and sulfur. In some embodiments, R^{A} is an optionally substituted 5-6 membered heteroaryl ring having 1-4 heteroatoms independently selected from nitrogen, oxygen, and sulfur.

In some embodiments, R^{A} is C₁₋₆ alkyl (e.g., methyl, ethyl, isopropyl). In some embodiments, R^{A} is C₁₋₆ haloalkyl (e.g., —CF₃, —CHF₂).

In some embodiment, R^{A} is as depicted in the compounds of Table 1, below.

As described above and defined herein, each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom are optionally taken together with their intervening atoms to form an optionally substituted 3-7 membered saturated or partially unsaturated ring having 0-3 heteroatoms, in addition to the atom to which they are attached, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 4-7 membered saturated or partially unsaturated carbocyclic. In some embodiments, R is an optionally substituted 4-7 membered saturated or partially unsaturated heterocyclic ring having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur. In some embodiments, two R groups on the same atom are optionally taken together with their intervening atoms to form optionally substituted 3-7 membered saturated or partially unsaturated ring having 0-3 heteroatoms, in addition to the atom to which they are attached, independently selected from nitrogen, oxygen, and sulfur.

In some embodiment, R is as depicted in the compounds of Table 1, below.

As described above and defined herein, each of X¹ and X² is independently a is a covalent bond, —CR₂—, —CR(OR)—, —CRF—, —CF₂—, —NR—, —O—, —S—, or —S(O)₂—.

In some embodiments, X¹ and/or X² is a covalent bond. In some embodiments, X¹ and/or X² is —CR₂—. In some embodiments, X¹ and/or X² is —CR(OR)—. In some embodiments, X¹ and/or X² is —CRF—. In some embodiments, X¹ and/or X² is —CF₂—. In some embodiments, X¹ and/or X² is —NR—. In some embodiments, X¹ and/or X² is —O—. In some embodiments, X¹ and/or X² is —S—. In some embodiments, X¹ and/or X² is —S(O)₂—.

In some embodiments, X¹ is a covalent bond, —NH—, or —NMe-.

In some embodiments, X² is a covalent bond, —CH₂—, —CMe(OMe)-, —CMe(F)—, —CMe(CF₃)—, cyclopropylenyl, difluorocyclopropylenyl, —NH—, —NMe-, —N(COMe)-, —N(CF₃)—, —NEt-, —N(nPr)—, —N(nBu)-, —N(Ph)-, —N(3-pyridyl)-, —N(4-pyridyl)-, —N(SO₂Me)-, —N(CH₂CHF₂)—, —N(CH₂cyclopropyl)-, —N(CH₂Ph)-, —N(CH₂CONH₂)—, —N(CH₂SO₂Me)-, —N(CH₂CH₂CHF₂)—, —N(CH₂CH₂Ph)-, —N(CH₂CH₂CO₂H)—, —N(CH₂CH₂CONH₂)—, —N(CH₂CH₂CN)—, —N(CH₂CH₂OMe)-, —N(CH₂CH₂SO₂Me)-, —O—, —S—, or —S(O)₂—.

In some embodiment, X is as depicted in the compounds of Table 1, below.

As described above and defined herein, Y¹ is a C₁₋₃ hydrocarbon chain wherein each methylene is optionally substituted with —CR₂—, —CR(OR)—, —C(O)—, —C(NR)—, —C(NOR)—, —S(O)—, or —S(O)₂—.

In some embodiments, Y¹ is a C₁₋₃ hydrocarbon chain wherein each methylene is optionally substituted with —CR₂—, —CR(OR)—, —C(O)—, —C(NR)—, —C(NOR)—, —S(O)—, or —S(O)₂—.

In some embodiments, Y¹ is a C₁₋₃ hydrocarbon chain. In some embodiments, Y¹ is —CR₂—. In some embodiments, Y¹ is —CR(OR)—. In some embodiments, Y¹ is —C(O)—. In some embodiments, Y¹ is —C(NR)—. In some embodiments, Y¹ is —C(NOR)—. In some embodiments, Y¹ is —S(O)—. In some embodiments, Y¹ is —S(O)₂—.

In some embodiments, Y¹ is —CH₂—, —CH₂C(O)—, —NHCH₂C(O)—, —CH₂CH₂C(O)—, —CH₂CH(OH)C(O)—, —C(O)—, —C(NH)—, —C(NOH)—, —S(O)—, or —S(O)₂—.

In some embodiment, Y¹ is as depicted in the compounds of Table 1, below.

As described above and defined herein, s is 0 or 1.

In some embodiments, s is 0. In some embodiments, s is 1.

In some embodiment, s is as depicted in the compounds of Table 1, below.

As described above and defined herein, each of t, u, v, w, x, y, and z are independently 0, 1, 2, 3, or 4.

In some embodiments, t is 0. In some embodiments, t is 1. In some embodiments, t is 2. In some embodiments, t is 3. In some embodiments, t is 4.

In some embodiments, u is 0. In some embodiments, u is 1. In some embodiments, u is 2. In some embodiments, u is 3. In some embodiments, u is 4.

In some embodiments, v is 0. In some embodiments, v is 1. In some embodiments, v is 2. In some embodiments, v is 3. In some embodiments, v is 4.

In some embodiments, w is 0. In some embodiments, w is 1. In some embodiments, w is 2. In some embodiments, w is 3. In some embodiments, w is 4.

In some embodiments, x is 0. In some embodiments, x is 1. In some embodiments, x is 2. In some embodiments, x is 3. In some embodiments, x is 4.

In some embodiments, y is 0. In some embodiments, y is 1. In some embodiments, y is 2. In some embodiments, y is 3. In some embodiments, y is 4.

In some embodiments, z is 0. In some embodiments, z is 1. In some embodiments, z is 2. In some embodiments, z is 3. In some embodiments, z is 4.

In some embodiment, t, u, v, w, x, y, and z are as depicted in the compounds of Table 1, below.

In some embodiments, DBM is

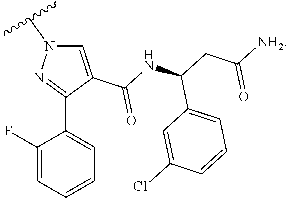 [SMILES: *n1cc(C(=O)N[C@@H](CC(N)=O)c2cccc(Cl)c2)c(-c2ccccc2F)n1]

In some embodiments, DBM is

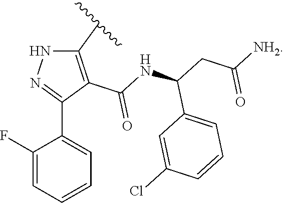 [SMILES: *c1[nH]nc(-c2ccccc2F)c1C(=O)N[C@@H](CC(N)=O)c1cccc(Cl)c1]

In some embodiments, DBM is

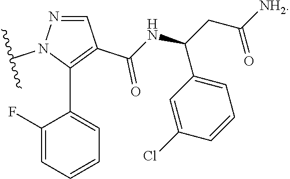 [SMILES: *n1ncc(C(=O)N[C@@H](CC(N)=O)c2cccc(Cl)c2)c1-c1ccccc1F]

In some embodiments, DBM is

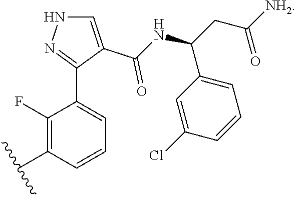 [SMILES: *c1cccc(-c2n[nH]cc2C(=O)N[C@@H](CC(N)=O)c2cccc(Cl)c2)c1F]

In some embodiments, DBM is

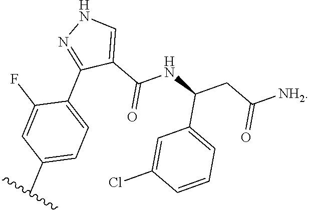 [SMILES: *c1ccc(-c2n[nH]cc2C(=O)N[C@@H](CC(N)=O)c2cccc(Cl)c2)c(F)c1]

In some embodiments, DBM is

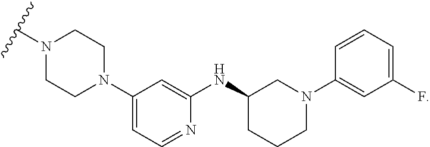 [SMILES: *N1CCN(c2ccnc(N[C@@H]3CCCN(c4cccc(F)c4)C3)c2)CC1]

In some embodiments, DBM is

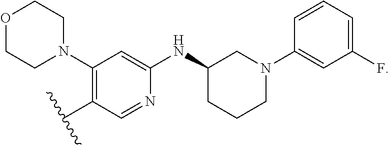 [SMILES: *c1cnc(N[C@@H]2CCCN(c3cccc(F)c3)C2)cc1N1CCOCC1]

In some embodiments, DBM is

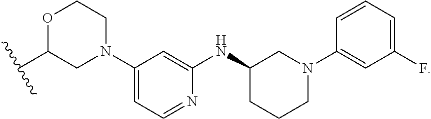 [SMILES: *C1CN(c2ccnc(N[C@@H]3CCCN(c4cccc(F)c4)C3)c2)CCO1]

In some embodiments, DBM is

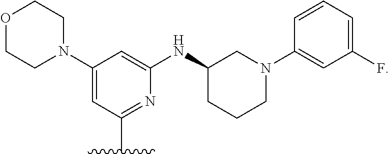 [SMILES: *c1cc(N2CCOCC2)cc(N[C@@H]2CCCN(c3cccc(F)c3)C2)n1]

In certain embodiments, the present invention provides a compound of Formula I-k-2-a represented by any one of the following formulae:

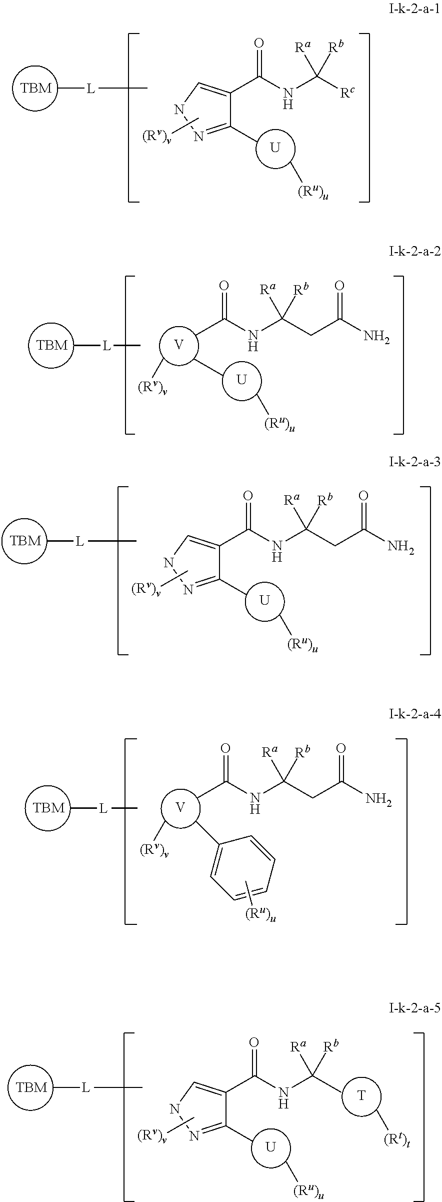

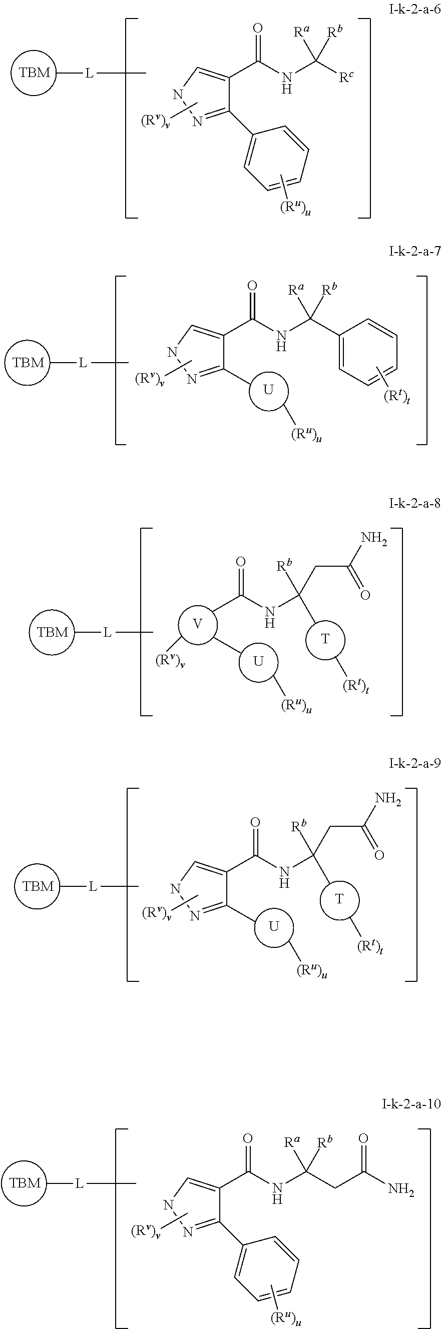

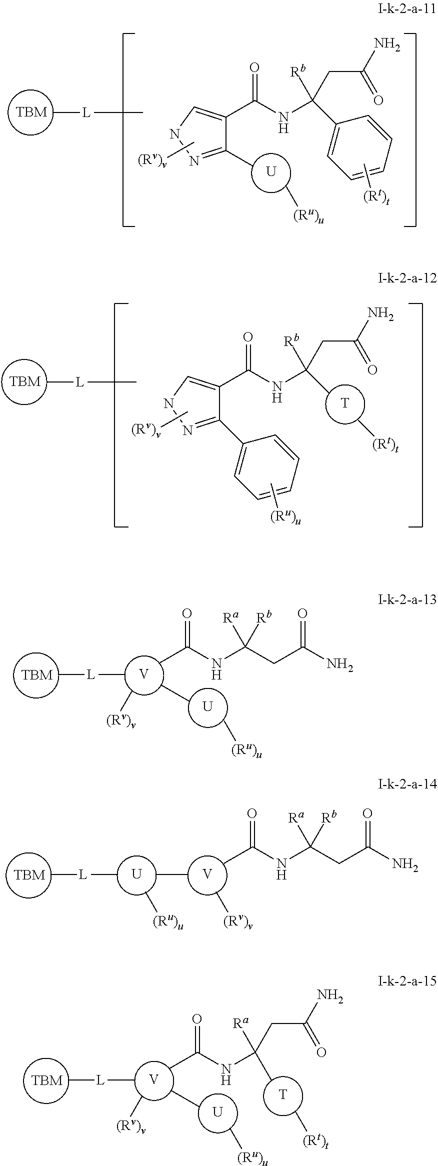

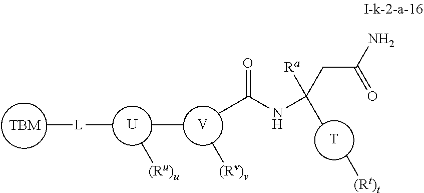

or a pharmaceutically acceptable salt thereof.

In certain embodiments, the present invention provides a compound of Formula I-k-2-b represented by any one of the following formulae:

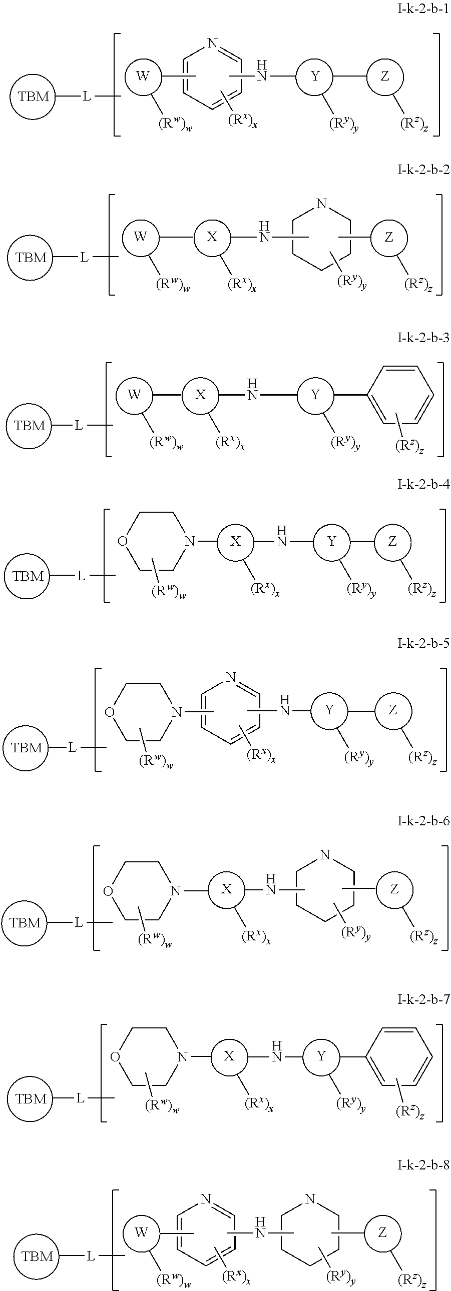

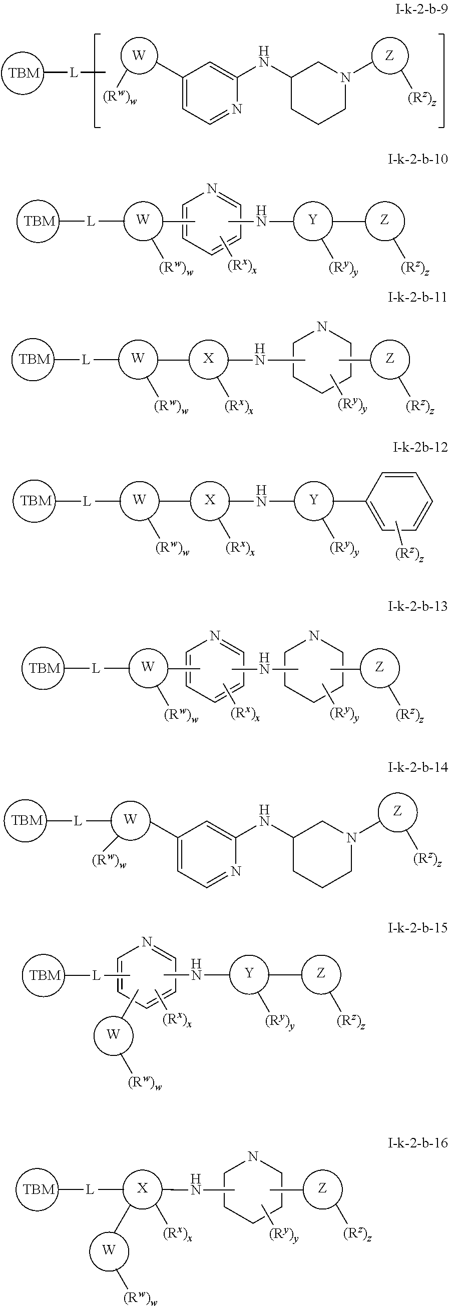

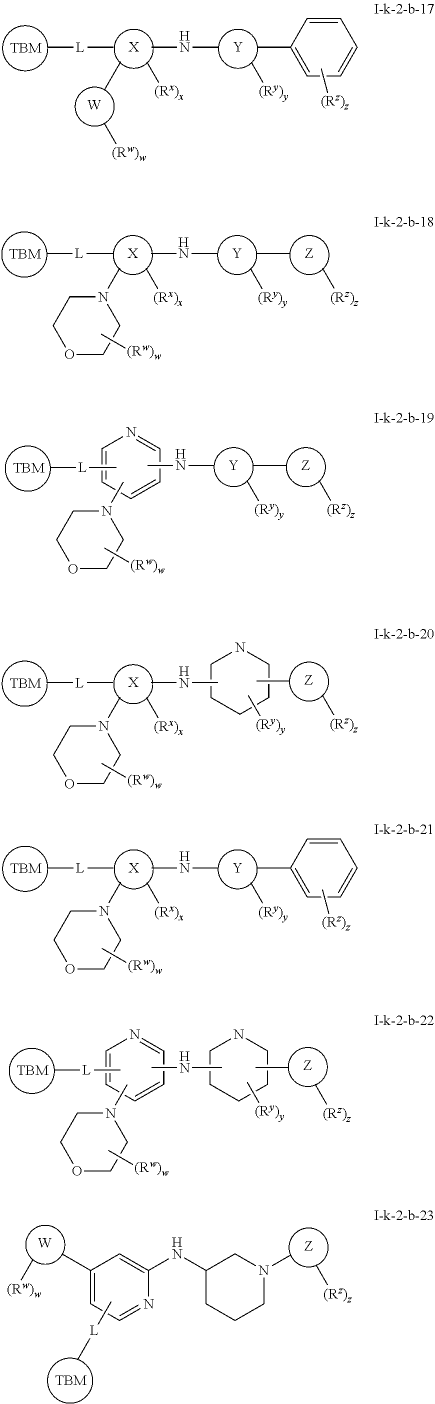

or a pharmaceutically acceptable salt thereof.

As defined above and described herein, DBM is further optionally substituted with

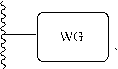

wherein

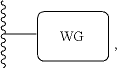

is a warhead group.

In some embodiments, the warhead group is -L²-Y, wherein:
L² is a covalent bond or a bivalent C₁₋₈ saturated or unsaturated, straight or branched, hydrocarbon chain, wherein one, two, or three methylene units of L² are optionally and independently replaced by cyclopropylene, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—;
Y is hydrogen, C₁₋₆ aliphatic optionally substituted with oxo, halogen, NO₂, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with 1-4 R^{e} groups; and
each R^{e} is independently selected from -Q-Z, oxo, NO₂, halogen, CN, a suitable leaving group, or a C₁₋₆ aliphatic optionally substituted with oxo, halogen, NO₂, or CN, wherein:
Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, or —SO₂—, —N(R)C(O)—, —C(O)N(R)—, —N(R)SO₂—, or —SO₂N(R)—; and
Z is hydrogen or C₁₋₆ aliphatic optionally substituted with oxo, halogen, NO₂, or CN.

In certain embodiments, L² is a covalent bond.

In certain embodiments, L² is a bivalent C₁₋₈ saturated or unsaturated, straight or branched, hydrocarbon chain. In certain embodiments, L² is —CH₂—.

In certain embodiments, L² is a covalent bond, —CH₂—, —NH—, —CH₂NH—, —NHCH₂—, —NHC(O)—, —NHC(O)CH₂OC(O)—, —CH₂NHC(O)—, —NHSO₂—, —NHSO₂CH₂—, —NHC(O)CH₂OC(O)—, or —SO₂NH—.

In some embodiments, L² is a bivalent C₂₋₈ straight or branched, hydrocarbon chain wherein L² has at least one double bond and one or two additional methylene units of L² are optionally and independently replaced by —NRC(O)—, —C(O)NR—, —N(R)SO₂—, —SO₂N(R)—, —S—, —S(O)—, —SO₂—, —OC(O)—, —C(O)O—, cyclopropylene, —O—, —N(R)—, or —C(O)—.

In certain embodiments, L² is a bivalent C₂₋₈ straight or branched, hydrocarbon chain wherein L² has at least one double bond and at least one methylene unit of L² is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L² are optionally and independently replaced by cyclopropylene, —O—, —N(R)—, or —C(O)—.

In some embodiments, L² is a bivalent C₂₋₈ straight or branched, hydrocarbon chain wherein L² has at least one double bond and at least one methylene unit of L² is replaced by —C(O)—, and one or two additional methylene units of L² are optionally and independently replaced by cyclopropylene, —O—, —N(R)—, or —C(O)—.

As described above, in certain embodiments, L² is a bivalent C₂₋₈ straight or branched, hydrocarbon chain wherein L² has at least one double bond. One of ordinary skill in the art will recognize that such a double bond may exist within the hydrocarbon chain backbone or may be “exo” to the backbone chain and thus forming an alkylidene group. By way of example, such an L² group having an alkylidene branched chain includes —CH₂C(═CH₂)CH₂—. Thus, in some embodiments, L² is a bivalent C₂₋₈ straight or branched, hydrocarbon chain wherein L² has at least one alkylidenyl double bond. Exemplary L² groups include —NHC(O)C(═CH₂)CH₂—.

In certain embodiments, L² is a bivalent C₂₋₈ straight or branched, hydrocarbon chain wherein L² has at least one double bond and at least one methylene unit of L² is replaced by —C(O)—. In certain embodiments, L² is —C(O)CH═CH(CH₃)—, —C(O)CH═CHCH₂NH(CH₃)—, —C(O)CH═CH(CH₃)—, —C(O)CH═CH—, —CH₂C(O)CH═CH—, —CH₂C(O)CH═CH(CH₃)—, —CH₂CH₂C(O)CH═CH—, —CH₂CH₂C(O)CH═CHCH₂—, —CH₂CH₂C(O)CH═CHCH₂NH(CH₃)—, or —CH₂CH₂C(O)CH═CH(CH₃)—, or —CH(CH₃)OC(O)CH═CH—.

In certain embodiments, L² is a bivalent C₂₋₈ straight or branched, hydrocarbon chain wherein L² has at least one double bond and at least one methylene unit of L² is replaced by —OC(O)—.

In some embodiments, L² is a bivalent C₂₋₈ straight or branched, hydrocarbon chain wherein L² has at least one double bond and at least one methylene unit of L² is replaced by —NRC(O)—, —C(O)NR—, —N(R)SO₂—, —SO₂N(R)—, —S—, —S(O)—, —SO₂—, —OC(O)—, or —C(O)O—, and one or two additional methylene units of L² are optionally and independently replaced by cyclopropylene, —O—, —N(R)—, or —C(O)—. In some embodiments, L² is —CH₂OC(O)CH═CHCH₂—, —CH₂—OC(O)CH═CH—, or —CH(CH═CH₂)OC(O)CH═CH—.

In certain embodiments, L² is —NRC(O)CH═CH—, —NRC(O)CH═CHCH₂N(CH₃)—, —NRC(O)CH═CHCH₂O—, —CH₂NRC(O)CH═CH—, —NRSO₂CH═CH—, —NRSO₂CH═CHCH₂—, —NRC(O)(C═N₂)C(O)—, —NRC(O)CH═CHCH₂N(CH₃)—, —NRSO₂CH═CH—, —NRSO₂CH═CHCH₂—, —NRC(O)CH═CHCH₂O—, —NRC(O)C(═CH₂)CH₂—, —CH₂NRC(O)—, —CH₂NRC(O)CH═CH—, —CH₂CH₂NRC(O)—, or —CH₂NRC(O)cyclopropylene-, wherein each R is independently hydrogen or optionally substituted C₁₋₆ aliphatic.

In certain embodiments, L² is —NHC(O)CH═CH—, —NHC(O)CH═CHCH₂N(CH₃)—, —NHC(O)CH═CHCH₂O—, —CH₂NHC(O)CH═CH—, —NHSO₂CH═CH—, —NHSO₂CH═CHCH₂—, —NHC(O)(C═N₂)C(O)—, —NHC(O)CH═CHCH₂N(CH₃)—, —NHSO₂CH═CH—, —NHSO₂CH═CHCH₂—, —NHC(O)CH═CHCH₂O—, —NHC(O)C(═CH₂)CH₂—, —CH₂NHC(O)—, —CH₂NHC(O)CH═CH—, —CH₂CH₂NHC(O)—, or —CH₂NHC(O)cyclopropylene-.

In some embodiments, L² is a bivalent C₂₋₈ straight or branched, hydrocarbon chain wherein L² has at least one triple bond. In certain embodiments, L² is a bivalent C₂₋₈ straight or branched, hydrocarbon chain wherein L² has at least one triple bond and one or two additional methylene units of L² are optionally and independently replaced by —NRC(O)—, —C(O)NR—, —S—, —S(O)—, —SO₂—, —C(═S)—, —C(═NR)—, —O—, —N(R)—, or —C(O)—. In some embodiments, L² has at least one triple bond and at least one methylene unit of L² is replaced by —N(R)—, —N(R)C(O)—, —C(O)—, —C(O)O—, or —OC(O)—, or —O—.

Exemplary L² groups include —C≡C—, —C≡CCH₂N(isopropyl)-, —NHC(O)C≡CCH₂CH₂—, —CH₂—C≡C≡CH₂—, —C≡CCH₂O—, —CH₂C(O)C≡C—, —C(O)C≡C—, or —CH₂OC(═O)C≡C—.

In certain embodiments, L² is a bivalent C₂₋₈ straight or branched, hydrocarbon chain wherein one methylene unit of L² is replaced by cyclopropylene and one or two additional methylene units of L² are independently replaced by —C(O)—, —NRC(O)—, —C(O)NR—, —N(R)SO₂—, or —SO₂N(R)—. Exemplary L² groups include —NHC(O)-cyclopropylene-SO₂— and —NHC(O)-cyclopropylene-.

As defined generally above, Y is hydrogen, C₁₋₆ aliphatic optionally substituted with oxo, halogen, NO₂, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with at 1-4 R^{e} groups, each R^{e} is independently selected from -Q-Z, oxo, NO₂, halogen, CN, a suitable leaving group, or C₁₋₆ aliphatic, wherein Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, or —SO₂—, —N(R)C(O)—, —C(O)N(R)—, —N(R)SO₂—, or —SO₂N(R)—; and, Z is hydrogen or C₁₋₆ aliphatic optionally substituted with oxo, halogen, NO₂, or CN.

In certain embodiments, Y is hydrogen.

In certain embodiments, Y is C₁₋₆ aliphatic optionally substituted with oxo, halogen, NO₂, or CN. In some embodiments, Y is C₂₋₆alkenyl optionally substituted with oxo, halogen, NO₂, or CN. In other embodiments, Y is C₂₋₆alkynyl optionally substituted with oxo, halogen, NO₂, or CN. In some embodiments, Y is C₂₋₆alkenyl. In other embodiments, Y is C₂₋₄ alkynyl.

In other embodiments, Y is C₁₋₆ alkyl substituted with oxo, halogen, NO₂, or CN. Such Y groups include —CH₂F, —CH₂Cl, —CH₂CN, and —CH₂NO₂.

In certain embodiments, Y is a saturated 3-6 membered monocyclic ring having 0-3 heteroatoms independently selected from nitrogen, oxygen, or sulfur, wherein Y is substituted with 1-4 R^{e} groups, wherein each R^{e} is as defined above and described herein.

In some embodiments, Y is a saturated 3-4 membered heterocyclic ring having 1 heteroatom selected from oxygen or nitrogen wherein said ring is substituted with 1-2 R^{e} groups, wherein each R^{e} is as defined above and described herein. Exemplary such rings are epoxide and oxetane rings, wherein each ring is substituted with 1-2 R^{e} groups, wherein each R^{e} is as defined above and described herein.

In other embodiments, Y is a saturated 5-6 membered heterocyclic ring having 1-2 heteroatom selected from oxygen or nitrogen wherein said ring is substituted with 1-4 R^{e} groups, wherein each R^{e} is as defined above and described herein. Such rings include piperidine and pyrrolidine, wherein each ring is substituted with 1-4 R^{e} groups, wherein each R^{e} is as defined above and described herein. In certain embodiments, Y is

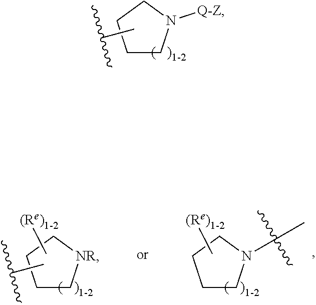

wherein each R, Q, Z, and R^{e} is as defined above and described herein.

In some embodiments, Y is a saturated 3-6 membered carbocyclic ring, wherein said ring is substituted with 1-4 R^{e} groups, wherein each R^{e} is as defined above and described herein. In certain embodiments, Y is cyclopropyl, cyclobutyl, cyclopentyl, or cyclohexyl, wherein each ring is substituted with 1-4 R^{e} groups, wherein each R^{e} is as defined above and described herein. In certain embodiments, Y is

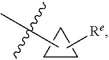

wherein R^{e} is as defined above and described herein.

In certain embodiments, Y is cyclopropyl optionally substituted with halogen, CN or NO₂.

In certain embodiments, Y is a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein.

In some embodiments, Y is a partially unsaturated 3-6 membered carbocyclic ring, wherein said ring is substituted with 1-4 R^{e} groups, wherein each R^{e} is as defined above and described herein. In some embodiments, Y is cyclopropenyl, cyclobutenyl, cyclopentenyl, or cyclohexenyl wherein each ring is substituted with 1-4 R^{e} groups, wherein each R^{e} is as defined 0-3 above and described herein. In certain embodiments, Y is

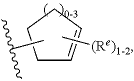

wherein each R^{e} is as defined above and described herein.

In certain embodiments, Y is a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein. In certain embodiments, Y is selected from:

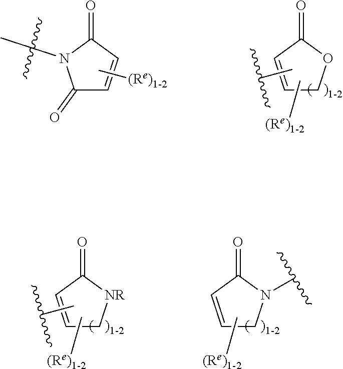

wherein each R and R^{e} is as defined above and described herein.

In certain embodiments, Y is a 6-membered aromatic ring having 0-2 nitrogens wherein said ring is substituted with 1-4 R^{e} groups, wherein each R^{e} group is as defined above and described herein. In certain embodiments, Y is phenyl, pyridyl, or pyrimidinyl, wherein each ring is substituted with 1-4 R^{e} groups, wherein each R^{e} is as defined above and described herein.

In some embodiments, Y is selected from:

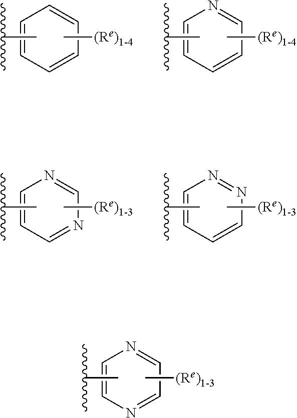

wherein each R^{e} is as defined above and described herein.

In other embodiments, Y is a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein. In some embodiments, Y is a 5 membered partially unsaturated or aryl ring having 1-3 heteroatoms independently selected from nitrogen, oxygen, and sulfur, wherein said ring is substituted with 1-4 R^{e} groups, wherein each R^{e} group is as defined above and described herein. Exemplary such rings are isoxazolyl, oxazolyl, thiazolyl, imidazolyl, pyrazolyl, pyrrolyl, furanyl, thienyl, triazole, thiadiazole, and oxadiazole, wherein each ring is substituted with 1-3 R^{e} groups, wherein each R^{e} group is as defined above and described herein. In certain embodiments, Y is selected from:

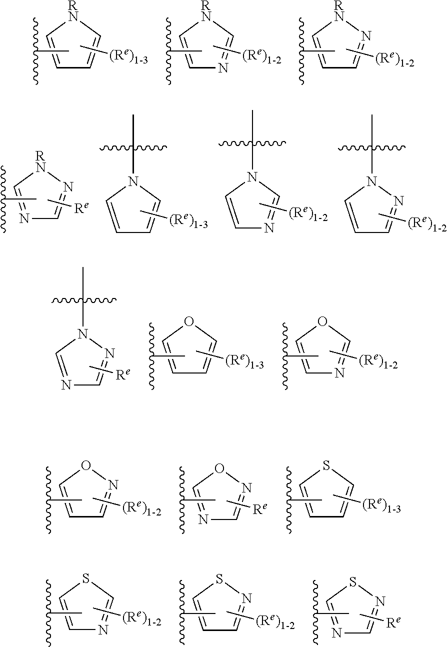

wherein each R and R^{e} is as defined above and described herein.

In certain embodiments, Y is an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein. According to another aspect, Y is a 9-10 membered bicyclic, partially unsaturated, or aryl ring having 1-3 heteroatoms independently selected from nitrogen, oxygen, or sulfur, wherein said ring is substituted with 1-4 R^{e} groups, wherein R^{e} is as defined above and described herein. Exemplary such bicyclic rings include 2,3-dihydrobenzo[d]isothiazole, wherein said ring is substituted with 1-4 R^{e} groups, wherein R^{e} is as defined above and described herein.

As defined generally above, each R^{e} group is independently selected from -Q-Z, oxo, NO₂, halogen, CN, a suitable leaving group, or C₁₋₆ aliphatic optionally substituted with oxo, halogen, NO₂, or CN, wherein Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, or —SO₂—, —N(R)C(O)—, —C(O)N(R)—, —N(R)SO₂—, or —SO₂N(R)—; and Z is hydrogen or C₁₋₆ aliphatic optionally substituted with oxo, halogen, NO₂, or CN.

In certain embodiments, R^{e} is C₁₋₆ aliphatic optionally substituted with oxo, halogen, NO₂, or CN. In other embodiments, R^{e} is oxo, NO₂, halogen, or CN.

In some embodiments, R^{e} is -Q-Z, wherein Q is a covalent bond and Z is hydrogen (i.e., R^{e} is hydrogen). In other embodiments, R^{e} is -Q-Z, wherein Q is a bivalent C₁₋₆ saturated or unsaturated, straight or branched, hydrocarbon chain, wherein one or two methylene units of Q are optionally and independently replaced by —NR—, —NRC(O)—, —C(O)NR—, —S—, —O—, —C(O)—, —SO—, or —SO₂—. In other embodiments, Q is a bivalent C₂₋₆ straight or branched, hydrocarbon chain having at least one double bond, wherein one or two methylene units of Q are optionally and independently replaced by —NR—, —NRC(O)—, —C(O)NR—, —S—, —O—, —C(O)—, —SO—, or —SO₂—. In certain embodiments, the Z moiety of the R^{e} group is hydrogen. In some embodiments, -Q-Z is —NHC(O)CH═CH₂ or —C(O)CH═CH₂.

In certain embodiments, each R^{e} is independently selected from oxo, NO₂, CN, fluoro, chloro, —NHC(O)CH═CH₂, —C(O)CH═CH₂, —CH₂CH═CH₂, —C—CH, —C(O)OCH₂Cl, —C(O)OCH₂F, —C(O)OCH₂CN, —C(O)CH₂Cl, —C(O)CH₂F, —C(O)CH₂CN, or —CH₂C(O)CH₃.

In certain embodiments, R^{e} is a suitable leaving group, i.e., a group that is subject to nucleophilic displacement. A “suitable leaving” is a chemical group that is readily displaced by a desired incoming chemical moiety such as the thiol moiety of a cysteine of interest. Suitable leaving groups are well known in the art, e.g., see, “Advanced Organic Chemistry,” Jerry March, 5^{th} Ed., pp. 351-357, John Wiley and Sons, N.Y. Such leaving groups include, but are not limited to, halogen, alkoxy, sulphonyloxy, optionally substituted alkylsulphonyloxy, optionally substituted alkenylsulfonyloxy, optionally substituted arylsulfonyloxy, acyl, and diazonium moieties. Examples of suitable leaving groups include chloro, iodo, bromo, fluoro, acetoxy, methanesulfonyloxy (mesyloxy), tosyloxy, triflyloxy, nitro-phenylsulfonyloxy (nosyloxy), and bromo-phenylsulfonyloxy (brosyloxy).

In certain embodiments, the following embodiments and combinations of -L²-Y apply:
(a) L² is a bivalent C₂₋₈ straight or branched, hydrocarbon chain wherein L² has at least one double bond and one or two additional methylene units of L² are optionally and independently replaced by —NRC(O)—, —C(O)NR—, —N(R)SO₂—, —SO₂N(R)—, —S—, —S(O)—, —SO₂—, —OC(O)—, —C(O)O—, cyclopropylene, —O—, —N(R)—, or —C(O)—; and Y is hydrogen or C₁₋₆ aliphatic optionally substituted with oxo, halogen, NO₂, or CN; or
(b) L² is a bivalent C₂₋₈ straight or branched, hydrocarbon chain wherein L² has at least one double bond and at least one methylene unit of L² is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L² are optionally and independently replaced by cyclopropylene, —O—, —N(R)—, or —C(O)—; and Y is hydrogen or C₁₋₆ aliphatic optionally substituted with oxo, halogen, NO₂, or CN; or
(c) L² is a bivalent C₂₋₈ straight or branched, hydrocarbon chain wherein L² has at least one double bond and at least one methylene unit of L² is replaced by —C(O)—, and one or two additional methylene units of L² are optionally and independently replaced by cyclopropylene, —O—, —N(R)—, or —C(O)—; and Y is hydrogen or C₁₋₆ aliphatic optionally substituted with oxo, halogen, NO₂, or CN; or
(d) L² is a bivalent C₂₋₈ straight or branched, hydrocarbon chain wherein L² has at least one double bond and at least one methylene unit of L² is replaced by —C(O)—; and Y is hydrogen or C₁₋₆ aliphatic optionally substituted with oxo, halogen, NO₂, or CN; or
(e) L² is a bivalent C₂₋₈ straight or branched, hydrocarbon chain wherein L² has at least one double bond and at least one methylene unit of L² is replaced by —OC(O)—; and Y is hydrogen or C₁₋₆ aliphatic optionally substituted with oxo, halogen, NO₂, or CN; or
(f) L² is —NRC(O)CH═CH—, —NRC(O)CH═CHCH₂N(CH₃)—, —NRC(O)CH═CHCH₂O—, —CH₂NRC(O)CH═CH—, —NRSO₂CH═CH—, —NRSO₂CH═CHCH₂—, —NRC(O)(C═N₂)—, —NRC(O)(C═N₂)C(O)—, —NRC(O)CH═CHCH₂N(CH₃)—, —NRSO₂CH═CH—, —NRSO₂CH═CHCH₂—, —NRC(O)CH═CHCH₂O—, —NRC(O)C(═CH₂)CH₂—, —CH₂NRC(O)—, —CH₂NRC(O)CH═CH—, —CH₂CH₂NRC(O)—, or —CH₂NRC(O)cyclopropylene-; wherein R is H or optionally substituted C₁₋₆ aliphatic; and Y is hydrogen or C₁₋₆ aliphatic optionally substituted with oxo, halogen, NO₂, or CN; or
(g) L² is —NHC(O)CH═CH—, —NHC(O)CH═CHCH₂N(CH₃)—, —NHC(O)CH═CHCH₂O—, —CH₂NHC(O)CH═CH—, —NHSO₂CH═CH—, —NHSO₂CH═CHCH₂—, —NHC(O)(C═N₂)—, —NHC(O)(C═N₂)C(O)—, —NHC(O)CH═CHCH₂N(CH₃)—, —NHSO₂CH═CH—, —NHSO₂CH═CHCH₂—, —NHC(O)CH═CHCH₂O—, —NHC(O)C(═CH₂)CH₂—, —CH₂NHC(O)—, —CH₂NHC(O)CH═CH—, —CH₂CH₂NHC(O)—, or —CH₂NHC(O)cyclopropylene-; and Y is hydrogen or C₁₋₆ aliphatic optionally substituted with oxo, halogen, NO₂, or CN; or
(h) L² is a bivalent C₂₋₈ straight or branched, hydrocarbon chain wherein L² has at least one alkylidenyl double bond and at least one methylene unit of L² is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L² are optionally and independently replaced by cyclopropylene, —O—, —N(R)—, or —C(O)—; and Y is hydrogen or C₁₋₆ aliphatic optionally substituted with oxo, halogen, NO₂, or CN; or
(i) L² is a bivalent C₂₋₈ straight or branched, hydrocarbon chain wherein L² has at least one triple bond and one or two additional methylene units of L² are optionally and independently replaced by —NRC(O)—, —C(O)NR—, —N(R)SO₂—, —SO₂N(R)—, —S—, —S(O)—, —SO₂—, —OC(O)—, or —C(O)O—, and Y is hydrogen or C₁₋₆ aliphatic optionally substituted with oxo, halogen, NO₂, or CN; or
(j) L² is —C≡C—, —C≡CCH₂N(isopropyl)-, —NHC(O)C≡CCH₂CH₂—, —CH₂—C≡C≡CH₂—, —C≡CCH₂O—, —CH₂C(O)C≡C—, —C(O)C≡C—, or —CH₂C(═O)C≡C—; and Y is hydrogen or C₁₋₆ aliphatic optionally substituted with oxo, halogen, NO₂, or CN; or
(k) L² is a bivalent C₂₋₈ straight or branched, hydrocarbon chain wherein one methylene unit of L² is replaced by cyclopropylene and one or two additional methylene units of L² are independently replaced by —NRC(O)—, —C(O)NR—, —N(R)SO₂—, —SO₂N(R)—, —S—, —S(O)—, —SO₂—, —OC(O)—, or —C(O)O—; and Y is hydrogen or C₁₋₆ aliphatic optionally substituted with oxo, halogen, NO₂, or CN; or
(l) L² is a covalent bond and Y is selected from:
   (i) C₁₋₆ alkyl substituted with oxo, halogen, NO₂, or CN;
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

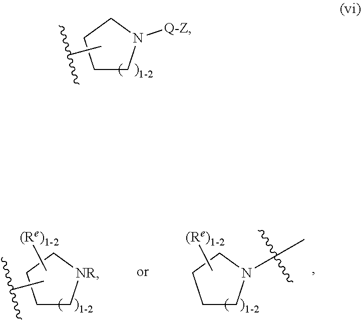

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or

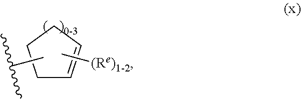

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

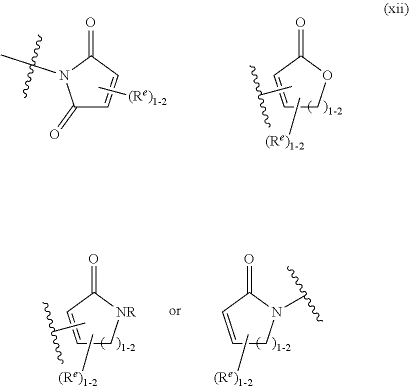

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

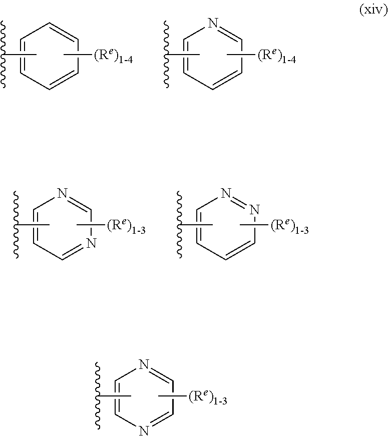

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

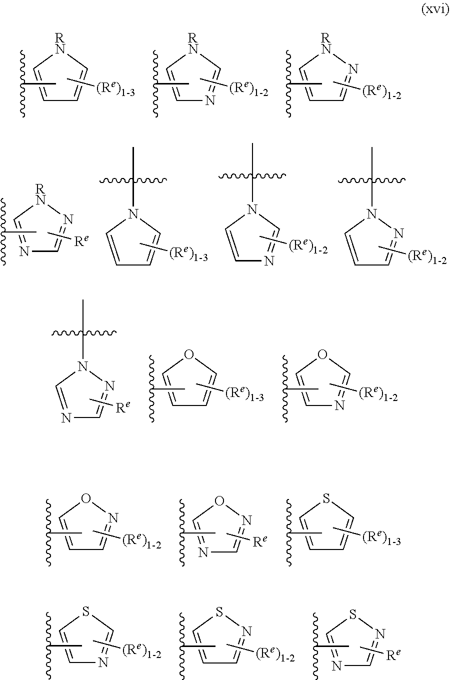

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(m) L² is —C(O)— and Y is selected from:
   (i) C₁₋₆ alkyl substituted with oxo, halogen, NO₂, or CN; or
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

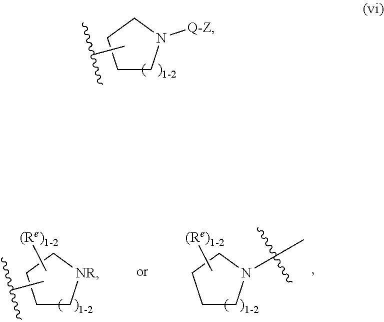

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or

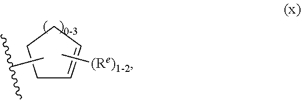

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

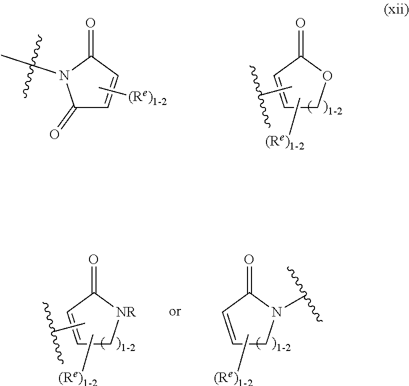

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

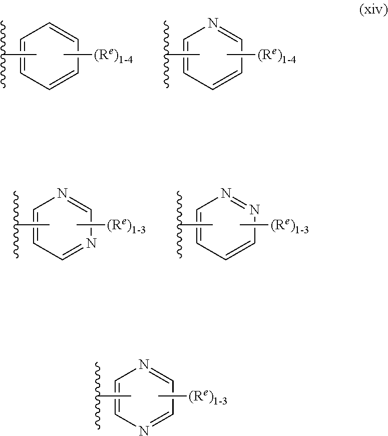

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

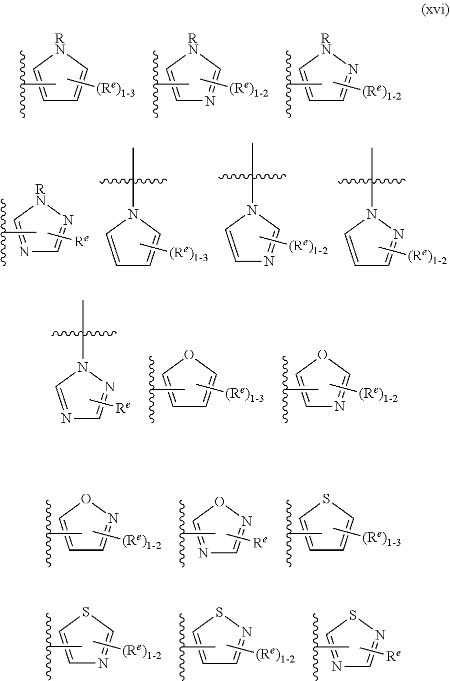

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(n) L² is —N(R)C(O)— and Y is selected from:
   (i) C₁₋₆ alkyl substituted with oxo, halogen, NO₂, or CN; or
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

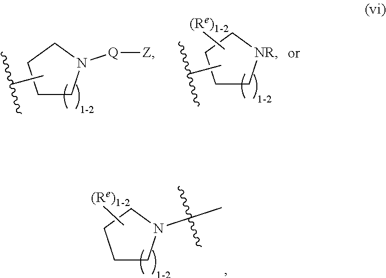

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or

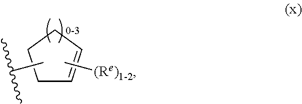

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

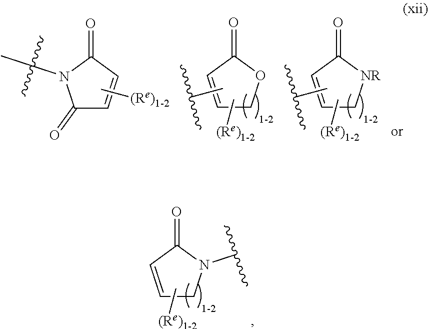

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

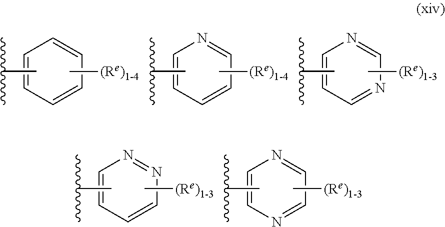

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

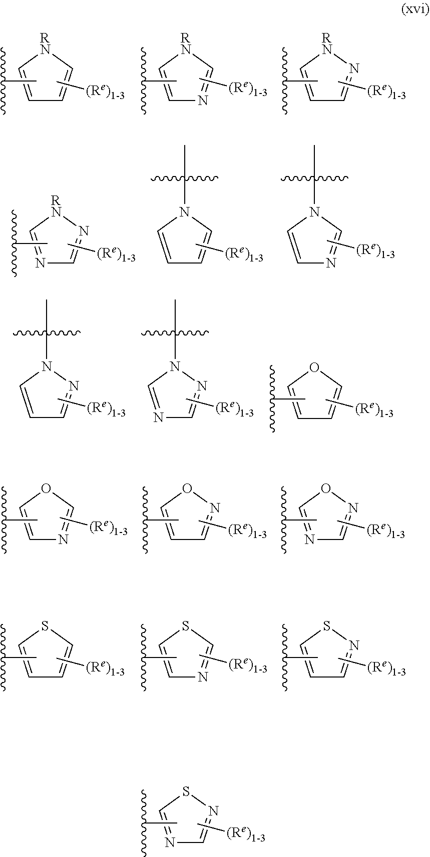

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(o) L² is a bivalent C₁₋₈ saturated or unsaturated, straight or branched, hydrocarbon chain; and Y is selected from:
   (i) C₁₋₆ alkyl substituted with oxo, halogen, NO₂, or CN;
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

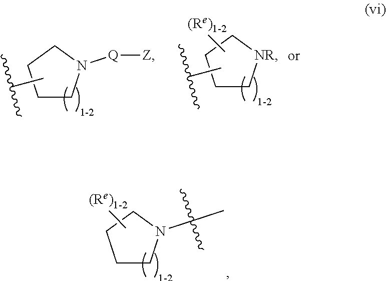

or wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or

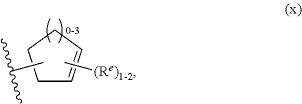

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

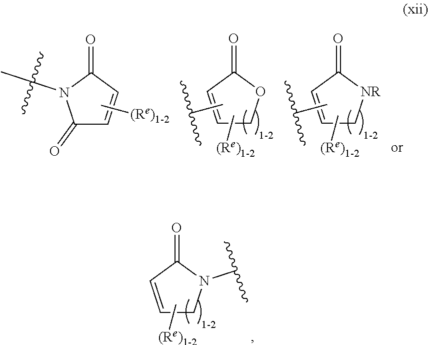

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

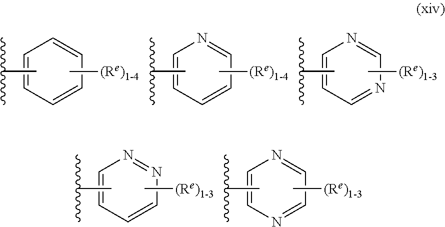

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

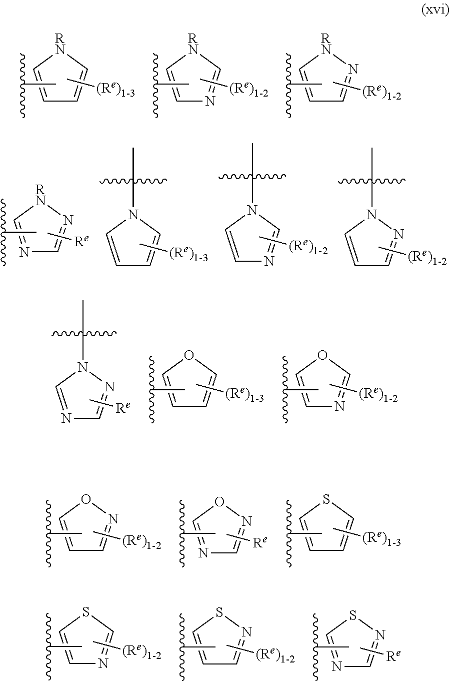

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(p) L² is a covalent bond, —CH₂—, —NH—, —C(O)—, —CH₂NH—, —NHCH₂—, —NHC(O)—, —NHC(O)CH₂OC(O)—, —CH₂NHC(O)—, —NHSO₂—, —NHSO₂CH₂—, —NHC(O)CH₂OC(O)—, or —SO₂NH—; and Y is selected from:
   (i) C₁₋₆ alkyl substituted with oxo, halogen, NO₂, or CN; or
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

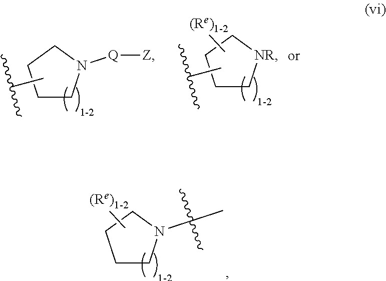

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or

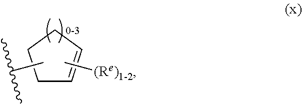

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

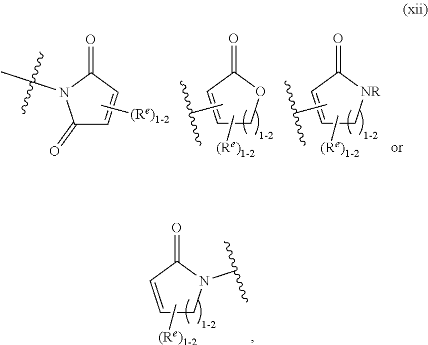

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

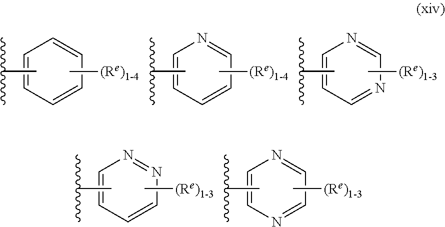

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

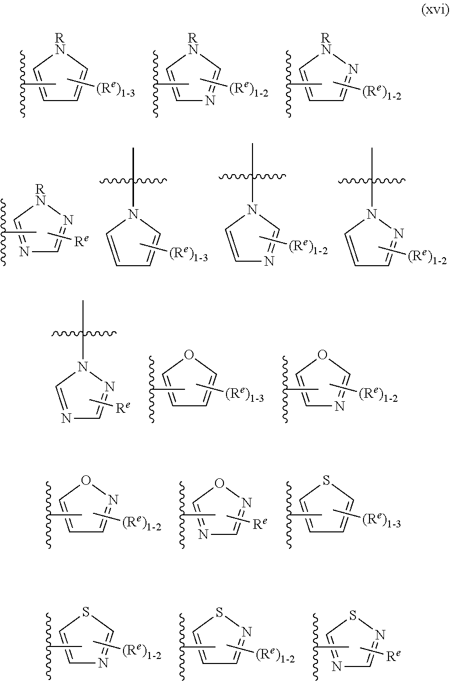

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein.

In certain embodiments, the Y group is selected from those set forth in Table 1A below, wherein each wavy line indicates the point of attachment to the rest of the molecule.

TABLE 1A
Exemplary Y Groups

| | |
|---|---|
| 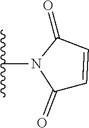 [SMILES: *N1C(=O)C=CC1=O] | a |
| 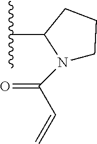 [SMILES: *C1CCCN1C(=O)C=C] | b |
| 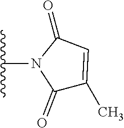 [SMILES: *N1C(=O)C=C(C)C1=O] | c |
| 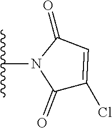 [SMILES: *N1C(=O)C=C(Cl)C1=O] | d |
| 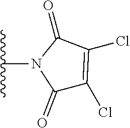 [SMILES: *N1C(=O)C(Cl)=C(Cl)C1=O] | e |
| 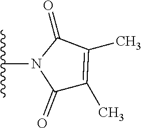 [SMILES: *N1C(=O)C(C)=C(C)C1=O] | f |
| 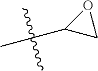 [SMILES: *C1CO1] | g |
| 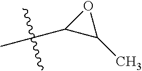 [SMILES: *C1OC1C] | h |
| 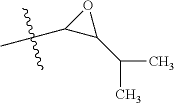 [SMILES: *C1OC1C(C)C] | i |
| 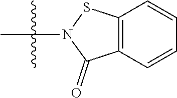 [SMILES: *n1sc2ccccc2c1=O] | j |
| 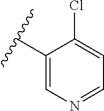 [SMILES: *c1cnccc1Cl] | k |
| 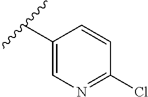 [SMILES: *c1ccc(Cl)nc1] | l |
| 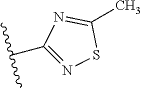 [SMILES: *c1nsc(C)n1] | m |
| 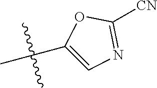 [SMILES: *c1cnc(C#N)o1] | n |
| 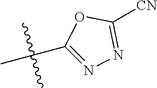 [SMILES: *c1nnc(C#N)o1] | o |
| 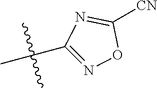 [SMILES: *c1noc(C#N)n1] | p |
| 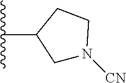 [SMILES: *C1CCN(C#N)C1] | q |
| 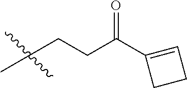 [SMILES: *CCC(=O)C1=CCC1] | r |
| 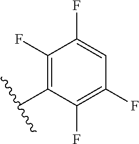 [SMILES: *c1c(F)c(F)cc(F)c1F] | s |
| 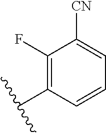 [SMILES: *c1cccc(C#N)c1F] | t |
| 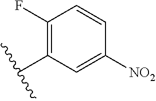 [SMILES: *c1cc([N+](=O)[O-])ccc1F] | u |
| 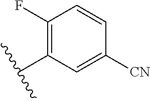 [SMILES: *c1cc(C#N)ccc1F] | v |
| 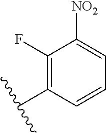 [SMILES: *c1cccc([N+](=O)[O-])c1F] | w |
| 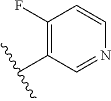 [SMILES: *c1cnccc1F] | x |
| 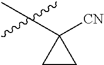 [SMILES: *C1(C#N)CC1] | y |
| 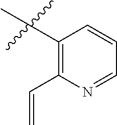 [SMILES: *c1cccnc1C=C] | z |
| 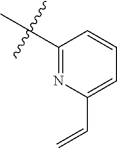 [SMILES: *c1cccc(C=C)n1] | aa |
| 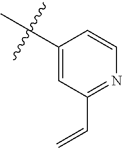 [SMILES: *c1ccnc(C=C)c1] | bb |
| 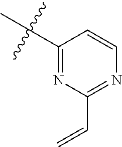 [SMILES: *c1ccnc(C=C)n1] | cc |
| 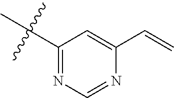 [SMILES: *c1cc(C=C)ncn1] | dd |
| 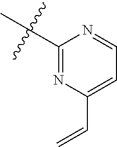 [SMILES: *c1nccc(C=C)n1] | ee |
| 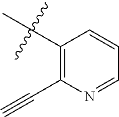 [SMILES: *c1cccnc1C#C] | ff |
| 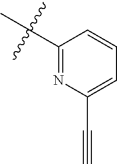 [SMILES: *c1cccc(C#C)n1] | gg |
| 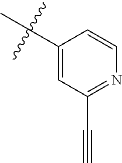 [SMILES: *c1ccnc(C#C)c1] | hh |
| 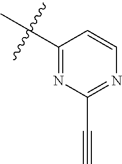 [SMILES: *c1ccnc(C#C)n1] | ii |
| 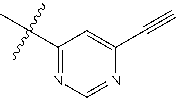 [SMILES: *c1cc(C#C)ncn1] | jj |
| 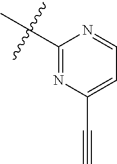 [SMILES: *c1nccc(C#C)n1] | kk |
| 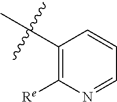 | ll |
| 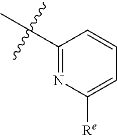 | mm |
| 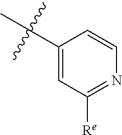 | nn |
| 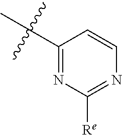 | oo |
| 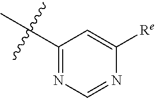 | pp |
| 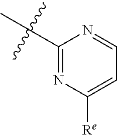 | qq |
| 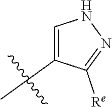 | rr |
| 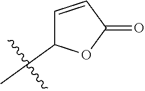 [SMILES: *C1C=CC(=O)O1] | ss |
| 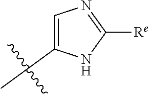 | tt |
| 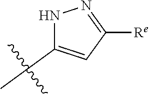 | uu |
| 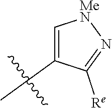 | vv |
| 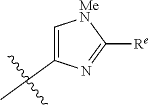 | ww |
| 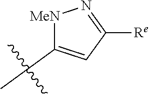 | xx |
| 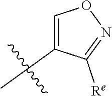 | yy |
| 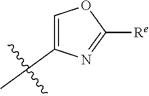 | zz |
| 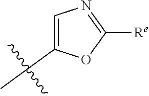 | aaa |
| 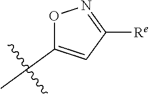 | bbb |
| 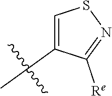 | ccc |
| 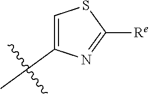 | ddd |
| 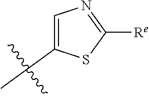 | eee |
| 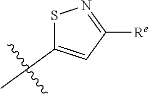 | fff |
| 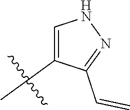 [SMILES: *c1c[nH]nc1C=C] | ggg |
| 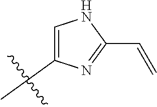 [SMILES: *c1c[nH]c(C=C)n1] | hhh |
| 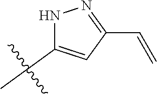 [SMILES: *c1cc(C=C)n[nH]1] | iii |
| 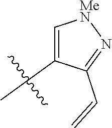 [SMILES: *c1cn(C)nc1C=C] | jjj |
| 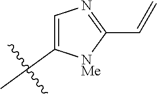 [SMILES: *c1cnc(C=C)n1C] | kkk |
| 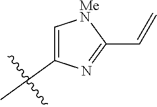 [SMILES: *c1cn(C)c(C=C)n1] | lll |
| 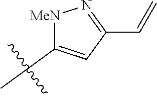 [SMILES: *c1cc(C=C)nn1C] | mmm |
| 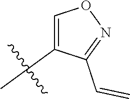 [SMILES: *c1conc1C=C] | nnn |
| 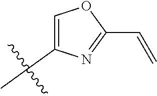 [SMILES: *c1coc(C=C)n1] | ooo |
| 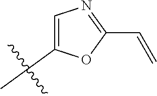 [SMILES: *c1cnc(C=C)o1] | ppp |
| 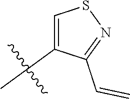 [SMILES: *c1csnc1C=C] | qqq |
| 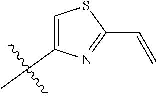 [SMILES: *c1csc(C=C)n1] | rrr |
| 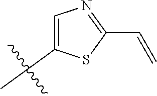 [SMILES: *c1cnc(C=C)s1] | sss |
| 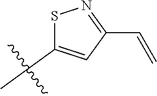 [SMILES: *c1cc(C=C)ns1] | ttt |
| 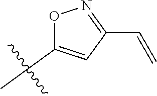 [SMILES: *c1cc(C=C)no1] | uuu |
| 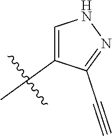 [SMILES: *c1c[nH]nc1C#C] | vvv |
| 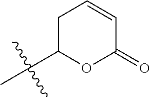 [SMILES: *C1CC=CC(=O)O1] | qqq |
| 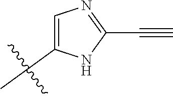 [SMILES: *c1cnc(C#C)[nH]1] | www |
| 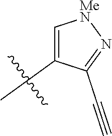 [SMILES: *c1cn(C)nc1C#C] | xxx |
| 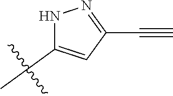 [SMILES: *c1cc(C#C)n[nH]1] | yyy |
| 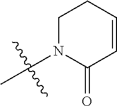 [SMILES: *N1CCC=CC1=O] | zzz |
| 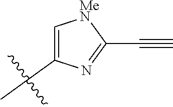 [SMILES: *c1cn(C)c(C#C)n1] | aaaa |
| 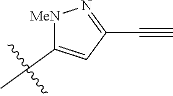 [SMILES: *c1cc(C#C)nn1C] | bbbb |
| 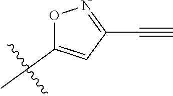 [SMILES: *c1cc(C#C)no1] | cccc |
| 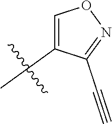 [SMILES: *c1conc1C#C] | dddd |
| 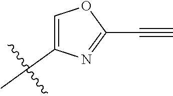 [SMILES: *c1coc(C#C)n1] | eeee |
| 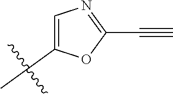 [SMILES: *c1cnc(C#C)o1] | ffff |
| 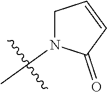 [SMILES: *N1CC=CC1=O] | gggg |
| 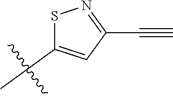 [SMILES: *c1cc(C#C)ns1] | hhhh |
| 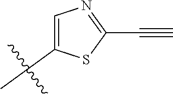 [SMILES: *c1cnc(C#C)s1] | iiii |
| 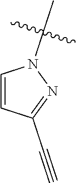 [SMILES: *n1ccc(C#C)n1] | jjjj |
| 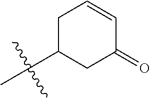 [SMILES: *C1CC=CC(=O)C1] | kkkk |
| 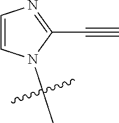 [SMILES: *n1ccnc1C#C] | llll |
| 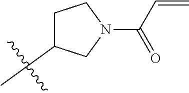 [SMILES: *C1CCN(C(=O)C=C)C1] | mmmm |
| 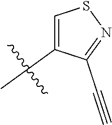 [SMILES: *c1csnc1C#C] | nnnn |
| 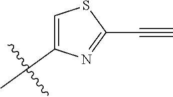 [SMILES: *c1csc(C#C)n1] | oooo |
| 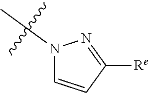 | pppp |
| 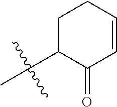 [SMILES: *C1CCC=CC1=O] | qqqq |
| 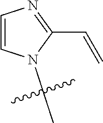 [SMILES: *n1ccnc1C=C] | rrrr |
| 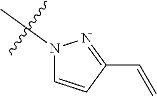 [SMILES: *n1ccc(C=C)n1] | ssss |
| 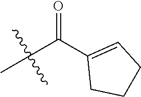 [SMILES: *C(=O)C1=CCCC1] | tttt |
| 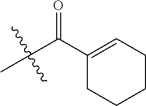 [SMILES: *C(=O)C1=CCCCC1] | uuuu |
| 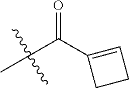 [SMILES: *C(=O)C1=CCC1] | vvvv |
| 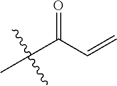 [SMILES: *C(=O)C=C] | wwww |
| 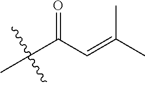 [SMILES: *C(=O)C=C(C)C] | xxxx |
| 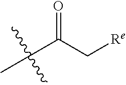 | yyyy |
| 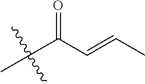 [SMILES: *C(=O)/C=C/C] | zzzz |
| 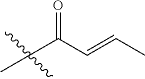 [SMILES: *C(=O)/C=C/C] | aaaaa |
| 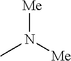 | bbbbb |
| 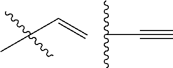 | ccccc |

wherein each R^{e} is independently a suitable leaving group, NO₂, CN or oxo.

In certain embodiments, a warhead group is —C≡CH, —C≡CCH₂NH(isopropyl), —NHC(O)C≡CCH₂CH₃, —CH₂—C≡C≡CH₃, —C≡CCH₂OH, —CH₂C(O)C≡CH, —C(O)C≡CH, or —CH₂C(═O)C≡CH. In some embodiments, R¹ is selected from —NHC(O)CH═CH₂, —NHC(O)CH═CHCH₂N(CH₃)₂, or —CH₂NHC(O)CH═CH₂.

In certain embodiments, a warhead group is selected from those set forth in Table 1B, below, wherein each wavy line indicates the point of attachment to the rest of the molecule.

TABLE 1B
Exemplary Warhead Groups

| | |
|---|---|
| 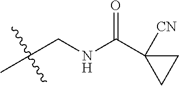 [SMILES: *CNC(=O)C1(C#N)CC1] | a |
| 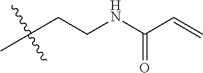 [SMILES: *CCNC(=O)C=C] | b |
| 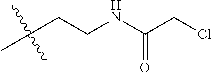 [SMILES: *CCNC(=O)CCl] | c |
| 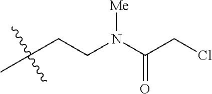 [SMILES: *CCN(C)C(=O)CCl] | d |
| 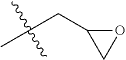 [SMILES: *CC1CO1] | e |
| 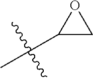 [SMILES: *C1CO1] | f |
| 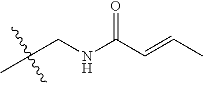 [SMILES: *CNC(=O)/C=C/C] | g |
| 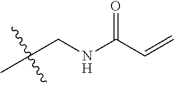 [SMILES: *CNC(=O)C=C] | h |
| 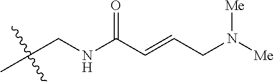 [SMILES: *CNC(=O)/C=C/CN(C)C] | i |
| 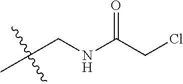 [SMILES: *CNC(=O)CCl] | j |
| 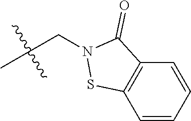 [SMILES: *Cn1sc2ccccc2c1=O] | k |
| 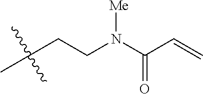 [SMILES: *CCN(C)C(=O)C=C] | l |
| 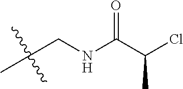 [SMILES: *CNC(=O)[C@H](C)Cl] | m |
| 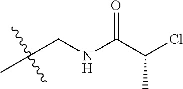 [SMILES: *CNC(=O)[C@@H](C)Cl] | n |
| 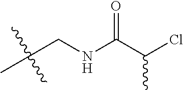 [SMILES: *CNC(=O)C(C)Cl] | o |
| 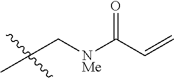 [SMILES: *CN(C)C(=O)C=C] | p |
| 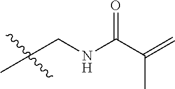 [SMILES: *CNC(=O)C(=C)C] | q |
| 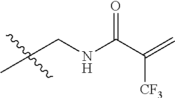 [SMILES: *CNC(=O)C(=C)C(F)(F)F] | r |
| 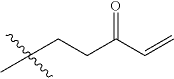 [SMILES: *CCC(=O)C=C] | s |
| 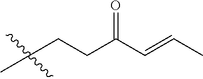 [SMILES: *CCC(=O)/C=C/C] | t |
| 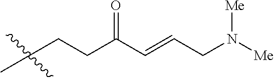 [SMILES: *CCC(=O)/C=C/CN(C)C] | u |
| 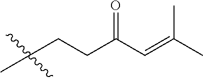 [SMILES: *CCC(=O)C=C(C)C] | v |
| 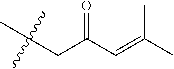 [SMILES: *CC(=O)C=C(C)C] | w |
| 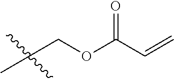 [SMILES: *COC(=O)C=C] | x |
| 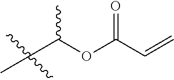 [SMILES: *C(C)OC(=O)C=C] | y |
| 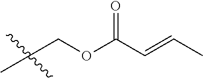 [SMILES: *COC(=O)/C=C/C] | z |
| 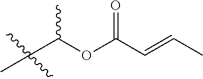 [SMILES: *C(C)OC(=O)/C=C/C] | aa |
| 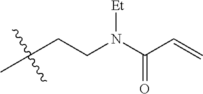 [SMILES: *CCN(CC)C(=O)C=C] | bb |
| 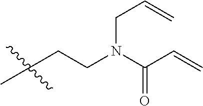 [SMILES: *CCN(CC=C)C(=O)C=C] | cc |
| 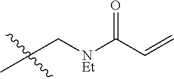 | dd |
| 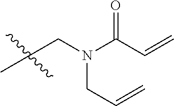 [SMILES: *CN(CC=C)C(=O)C=C] | ee |
| 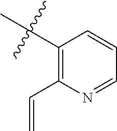 [SMILES: *c1cccnc1C=C] | ff |
| 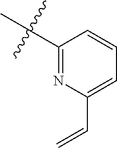 [SMILES: *c1cccc(C=C)n1] | gg |
| 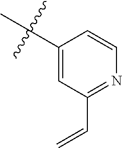 [SMILES: *c1ccnc(C=C)c1] | hh |
| 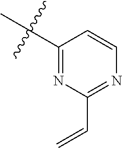 [SMILES: *c1ccnc(C=C)n1] | ii |
| 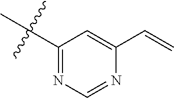 [SMILES: *c1cc(C=C)ncn1] | jj |
| 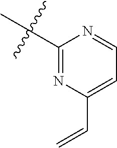 [SMILES: *c1nccc(C=C)n1] | kk |
| 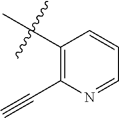 [SMILES: *c1cccnc1C#C] | ll |
| 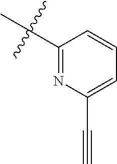 [SMILES: *c1cccc(C#C)n1] | mm |
| 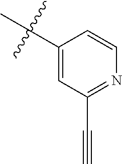 [SMILES: *c1ccnc(C#C)c1] | nn |
| 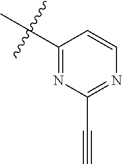 [SMILES: *c1ccnc(C#C)n1] | oo |
| 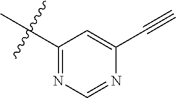 [SMILES: *c1cc(C#C)ncn1] | pp |
| 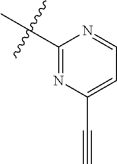 [SMILES: *c1nccc(C#C)n1] | qq |
| 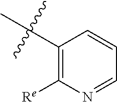 | rr |
| 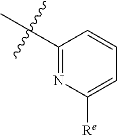 | ss |
| 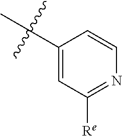 | tt |
| 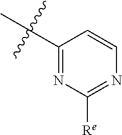 | uu |
| 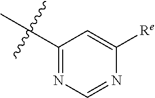 | vv |
| 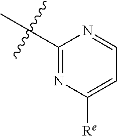 | ww |
| 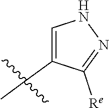 | xx |
| 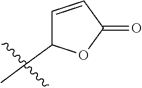 [SMILES: *C1C=CC(=O)O1] | yy |
| 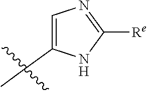 | zz |
| 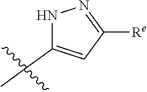 | aaa |
| 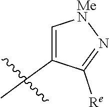 | bbb |
| 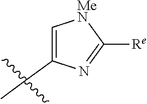 | ccc |
| 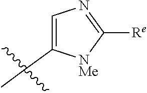 | ddd |
| 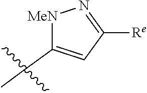 | eee |
| 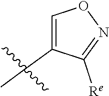 | fff |
| 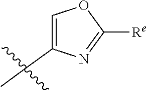 | ggg |
| 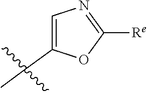 | hhh |
| 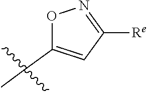 | iii |
| 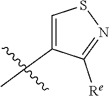 | jjj |
| 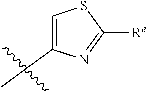 | kkk |
| 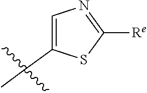 | lll |
| 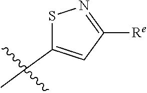 | mmm |
| 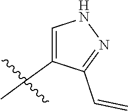 [SMILES: *c1c[nH]nc1C=C] | nnn |
| 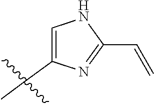 [SMILES: *c1c[nH]c(C=C)n1] | ooo |
| 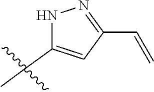 [SMILES: *c1cc(C=C)n[nH]1] | ppp |
| 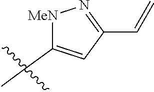 [SMILES: *c1cc(C=C)nn1C] | |
| 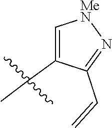 [SMILES: *c1cn(C)nc1C=C] | qqq |
| 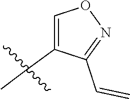 [SMILES: *c1conc1C=C] | rrr |
| 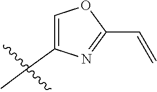 [SMILES: *c1coc(C=C)n1] | sss |
| 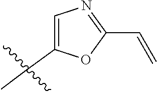 [SMILES: *c1cnc(C=C)o1] | ttt |
| 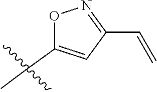 [SMILES: *c1cc(C=C)no1] | uuu |
| 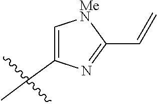 [SMILES: *c1cn(C)c(C=C)n1] | vvv |
| 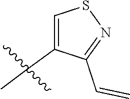 [SMILES: *c1csnc1C=C] | www |
| 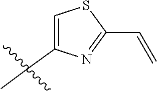 [SMILES: *c1csc(C=C)n1] | xxx |
| 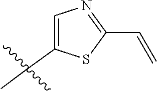 [SMILES: *c1cnc(C=C)s1] | yyy |
| 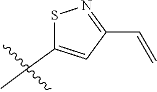 [SMILES: *c1cc(C=C)ns1] | zzz |
| 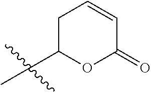 [SMILES: *C1CC=CC(=O)O1] | aaaa |
| 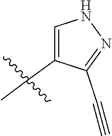 [SMILES: *c1c[nH]nc1C#C] | bbbb |
| 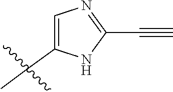 [SMILES: *c1cnc(C#C)[nH]1] | cccc |
| 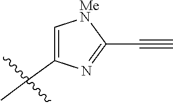 [SMILES: *c1cn(C)c(C#C)n1] | dddd |
| 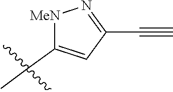 [SMILES: *c1cc(C#C)nn1C] | eeee |
| 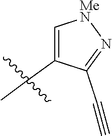 [SMILES: *c1cn(C)nc1C#C] | ffff |
| 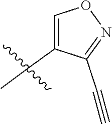 [SMILES: *c1conc1C#C] | gggg |
| 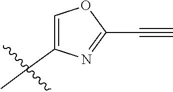 [SMILES: *c1coc(C#C)n1] | hhhh |
| 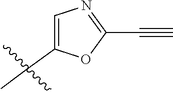 [SMILES: *c1cnc(C#C)o1] | iiii |
| 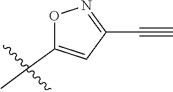 [SMILES: *c1cc(C#C)no1] | jjjj |
| 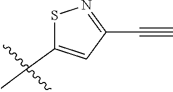 [SMILES: *c1cc(C#C)ns1] | kkkk |
| 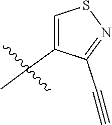 [SMILES: *c1csnc1C#C] | llll |
| 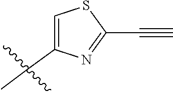 [SMILES: *c1csc(C#C)n1] | mmmm |
| 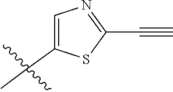 [SMILES: *c1cnc(C#C)s1] | nnnn |
| 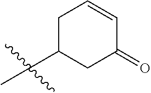 [SMILES: *C1CC=CC(=O)C1] | oooo |
| 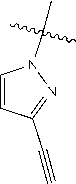 [SMILES: *n1ccc(C#C)n1] | pppp |
| 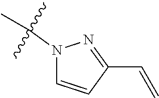 [SMILES: *n1ccc(C=C)n1] | qqqq |
| 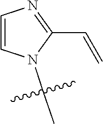 [SMILES: *n1ccnc1C=C] | rrrr |
| 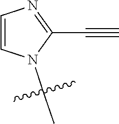 [SMILES: *n1ccnc1C#C] | ssss |
| 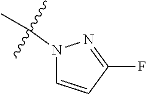 [SMILES: *n1ccc(F)n1] | tttt |
| 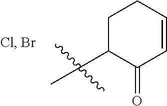 [SMILES: *C1CCC=CC1=O] | uuuu |
| 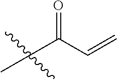 [SMILES: *C(=O)C=C] | vvvv |
| 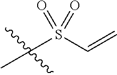 [SMILES: *S(=O)(=O)C=C] | wwww |
| 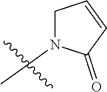 [SMILES: *N1CC=CC1=O] | xxxx |
| 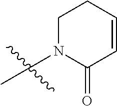 [SMILES: *N1CCC=CC1=O] | yyyy |
| 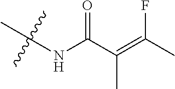 [SMILES: *NC(=O)/C(C)=C(/C)F] | zzzz |
| 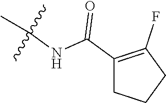 [SMILES: *NC(=O)C1=C(F)CCC1] | aaaaa |
| 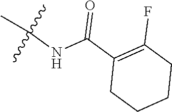 [SMILES: *NC(=O)C1=C(F)CCCC1] | bbbbb |
| 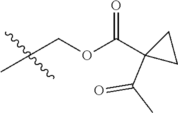 [SMILES: *COC(=O)C1(C(C)=O)CC1] | ccccc |
| 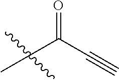 [SMILES: *C(=O)C#C] | ddddd |
| 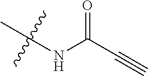 [SMILES: *NC(=O)C#C] | eeeee |
| 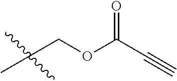 [SMILES: *COC(=O)C#C] | fffff |
| 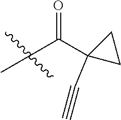 [SMILES: *C(=O)C1(C#C)CC1] | ggggg |
| 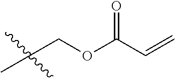 [SMILES: *COC(=O)C=C] | hhhhh |
| 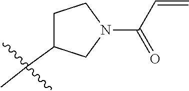 [SMILES: *C1CCN(C(=O)C=C)C1] | iiiii |
| 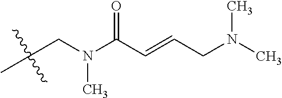 [SMILES: *CN(C)C(=O)/C=C/CN(C)C] | jjjjj |
| 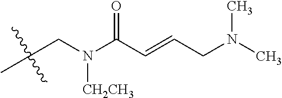 [SMILES: *CN(CC)C(=O)/C=C/CN(C)C] | kkkkk |
| 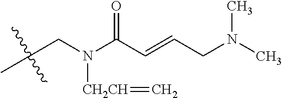 | lllll |
| 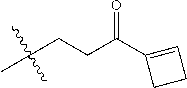 [SMILES: *CCC(=O)C1=CCC1] | mmmmm |
| 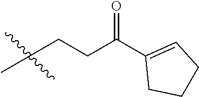 [SMILES: *CCC(=O)C1=CCCC1] | nnnnn |
| 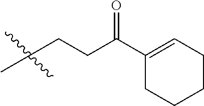 [SMILES: *CCC(=O)C1=CCCCC1] | ooooo |
| 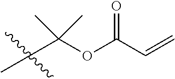 [SMILES: *C(C)(C)OC(=O)C=C] | ppppp |
| 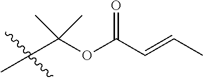 [SMILES: *C(C)(C)OC(=O)/C=C/C] | qqqqq |
| 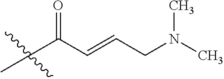 [SMILES: *C(=O)/C=C/CN(C)C] | rrrrr |
| 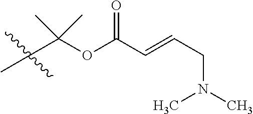 [SMILES: *C(C)(C)OC(=O)/C=C/CN(C)C] | sssss |
| 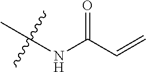 [SMILES: *NC(=O)C=C] | ttttt |
| 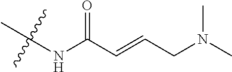 [SMILES: *NC(=O)/C=C/CN(C)C] | uuuuu |
| 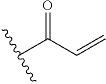 [SMILES: *C(=O)C=C] | vvvvv |
| 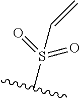 [SMILES: *S(=O)(=O)C=C] | wwwww |
| 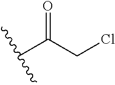 [SMILES: *C(=O)CCl] | xxxxx |
| 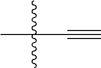 [SMILES: *C#C] | yyyyy |
| 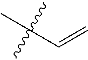 [SMILES: *C=C] | zzzzz |
| 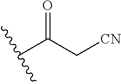 [SMILES: *C(=O)CC#N] | aaaaaa |
| 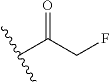 [SMILES: *C(=O)CF] | bbbbbb |
| 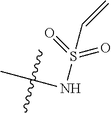 [SMILES: *NS(=O)(=O)C=C] | cccccc |
| 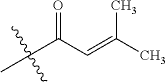 [SMILES: *C(=O)C=C(C)C] | dddddd |
| 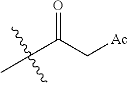 [SMILES: *C(=O)CC(C)=O] | eeeeee |
| 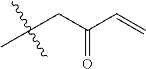 [SMILES: *CC(=O)C=C] | ffffff |
| 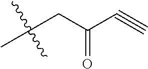 [SMILES: *CC(=O)C#C] | gggggg |
| 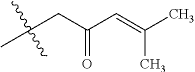 [SMILES: *CC(=O)C=C(C)C] | hhhhhh |
| 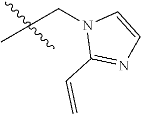 [SMILES: *Cn1ccnc1C=C] | iiiiii |
| 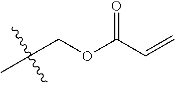 [SMILES: *COC(=O)C=C] | jjjjjj |
| 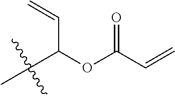 [SMILES: *C(C=C)OC(=O)C=C] | kkkkkk |
| 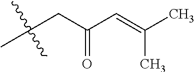 [SMILES: *CC(=O)C=C(C)C] | llllll |
| 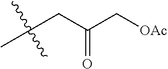 [SMILES: *CC(=O)COC(C)=O] | mmmmmm |
| 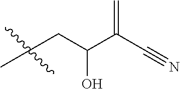 [SMILES: *CC(O)C(=C)C#N] | nnnnnn |
| 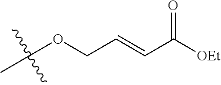 [SMILES: *OC/C=C/C(=O)OCC] | oooooo |
| 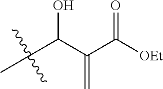 [SMILES: *C(O)C(=C)C(=O)OCC] | pppppp |
| 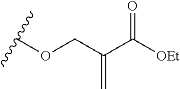 [SMILES: *OCC(=C)C(=O)OCC] | qqqqqq |
| 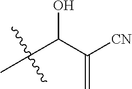 [SMILES: *C(O)C(=C)C#N] | rrrrrr |
| 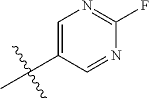 [SMILES: *c1cnc(F)nc1] | ssssss |
| 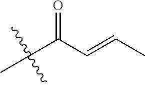 [SMILES: *C(=O)/C=C/C] | tttttt |
| 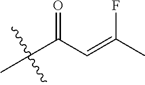 [SMILES: *C(=O)/C=C(/C)F] | uuuuuu |
| 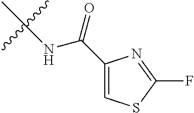 [SMILES: *NC(=O)c1csc(F)n1] | vvvvvv |
| 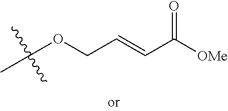 [SMILES: *OC/C=C/C(=O)OC] | wwwwww |
| 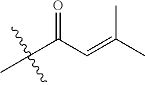 [SMILES: *C(=O)C=C(C)C] | xxxxxx |

wherein each R^{e} is independently a suitable leaving group, NO₂, CN, or oxo.

In some embodiments, Y of a warhead group is an isoxazoline compound or derivative capable of covalently binding to serine. In some embodiments, Y of a warhead group is an isoxazoline compound or derivative described in WO 2010135360, the entire content of which is incorporated herein by reference. As understood by one skilled in the art, an isoxazoline compound or derivative described in WO 2010135360, as Y of a warhead group, can covalently connect to L² of the warhead group at any reasonable position of the isoxazoline compound or derivative. In some embodiments, Y of a warhead group is:

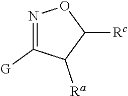

wherein G, R^{a}, and R^{c} are:

| G | R^{a} | R^{c} |
|---|---|---|
| —Br | —H | —H |
| —Cl | —H | —H |
| 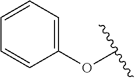 [SMILES: *Oc1ccccc1] | —H | —H |
| 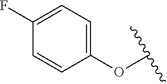 [SMILES: *Oc1ccc(F)cc1] | —H | —H |
| 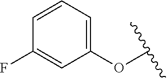 [SMILES: *Oc1cccc(F)c1] | —H | —H |
| 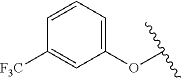 [SMILES: *Oc1cccc(C(F)(F)F)c1] | —H | —H |
| 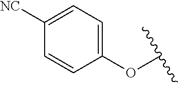 [SMILES: *Oc1ccc(C#N)cc1] | —H | —H |
| 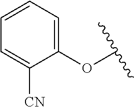 [SMILES: *Oc1ccccc1C#N] | —H | —H |
| 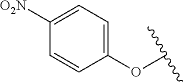 [SMILES: *Oc1ccc([N+](=O)[O-])cc1] | —H | —H |
| 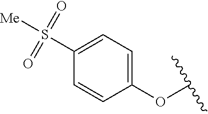 [SMILES: *Oc1ccc(S(C)(=O)=O)cc1] | —H | —H |
| 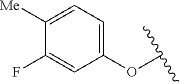 [SMILES: *Oc1ccc(C)c(F)c1] | —H | —H |
| 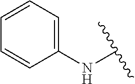 [SMILES: *Nc1ccccc1] | —H | —H |
| 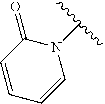 [SMILES: *n1ccccc1=O] | —H | —H |
| 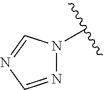 [SMILES: *n1cncn1] | —H | —H |
| 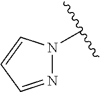 [SMILES: *n1cccn1] | —H | —H |
| 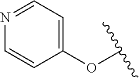 [SMILES: *Oc1ccncc1] | —H | —H |
| 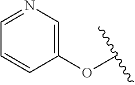 [SMILES: *Oc1cccnc1] | —H | —H |
| 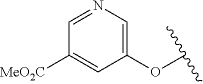 [SMILES: *Oc1cncc(C(=O)OC)c1] | —H | —H |
| 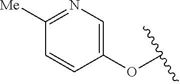 [SMILES: *Oc1ccc(C)nc1] | —II | —H |
| 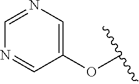 [SMILES: *Oc1cncnc1] | —H | —H |
| 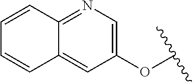 [SMILES: *Oc1cnc2ccccc2c1] | —H | —H |
| 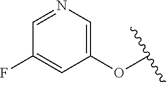 [SMILES: *Oc1cncc(F)c1] | —H | —H |
| 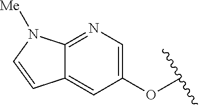 [SMILES: *Oc1cnc2c(ccn2C)c1] | —H | —H |
| —OMe | —H | —H |
| 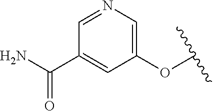 [SMILES: *Oc1cncc(C(N)=O)c1] | —H | —H |
| 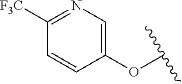 [SMILES: *Oc1ccc(C(F)(F)F)nc1] | —H | —H |
| 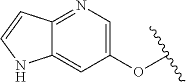 [SMILES: *Oc1cnc2cc[nH]c2c1] | —H | —H |
| 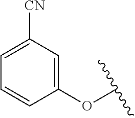 [SMILES: *Oc1cccc(C#N)c1] | —H | —H |
| 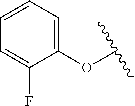 [SMILES: *Oc1ccccc1F] | —H | —H |
| 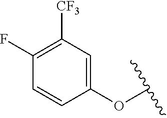 [SMILES: *Oc1ccc(F)c(C(F)(F)F)c1] | H~ | —H |
| 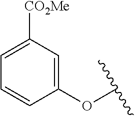 [SMILES: *Oc1cccc(C(=O)OC)c1] | —H | —H |
| 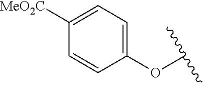 [SMILES: *Oc1ccc(C(=O)OC)cc1] | —H | —H |
| 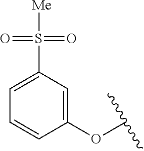 [SMILES: *Oc1cccc(S(C)(=O)=O)c1] | —H | —H |
| 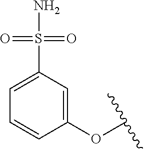 [SMILES: *Oc1cccc(S(N)(=O)=O)c1] | —H | —H |
| 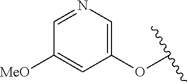 [SMILES: *Oc1cncc(OC)c1] | —H | —H |
| 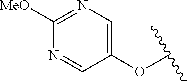 [SMILES: *Oc1cnc(OC)nc1] | —H | —H |
| 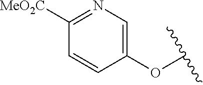 [SMILES: *Oc1ccc(C(=O)OC)nc1] | —H | —H |
| 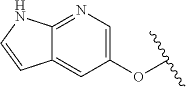 [SMILES: *Oc1cnc2[nH]ccc2c1] | —H | —H |
| 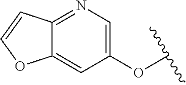 [SMILES: *Oc1cnc2ccoc2c1] | —H | —H |
| 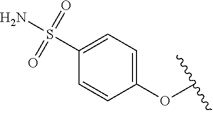 [SMILES: *Oc1ccc(S(N)(=O)=O)cc1] | —H | —H |
| 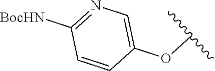 [SMILES: *Oc1ccc(NC(=O)OC(C)(C)C)nc1] | —H | —H |
| 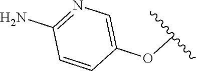 [SMILES: *Oc1ccc(N)nc1] | —H | —H |
| 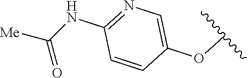 [SMILES: *Oc1ccc(NC(C)=O)nc1] | —H | —H |
| 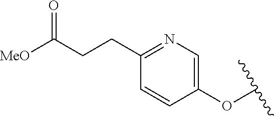 [SMILES: *Oc1ccc(CCC(=O)OC)nc1] | —H | —H |
| 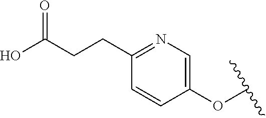 [SMILES: *Oc1ccc(CCC(=O)O)nc1] | —H | —H |
| 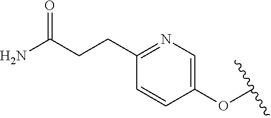 [SMILES: *Oc1ccc(CCC(N)=O)nc1] | —H | —H |
| 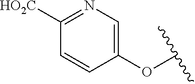 [SMILES: *Oc1ccc(C(=O)O)nc1] | —H | —H |
| 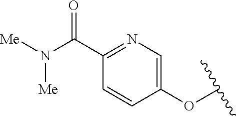 [SMILES: *Oc1ccc(C(=O)N(C)C)nc1] | —H | —H |
| 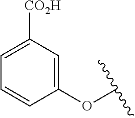 [SMILES: *Oc1cccc(C(=O)O)c1] | —H | —H |
| 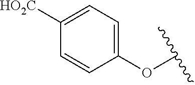 [SMILES: *Oc1ccc(C(=O)O)cc1] | —H | —H |
| 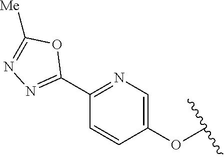 [SMILES: *Oc1ccc(-c2nnc(C)o2)nc1] | —H | —H |
| 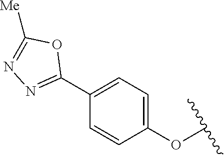 [SMILES: *Oc1ccc(-c2nnc(C)o2)cc1] | —H | —H |
| 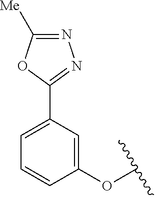 [SMILES: *Oc1cccc(-c2nnc(C)o2)c1] | —H | —H |
| 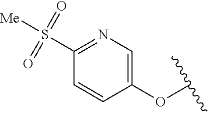 [SMILES: *Oc1ccc(S(C)(=O)=O)nc1] | —H | —H |
| 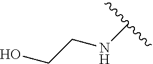 [SMILES: *NCCO] | —H | —H |
| 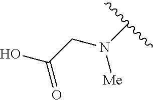 [SMILES: *N(C)CC(=O)O] | —H | —H |
| 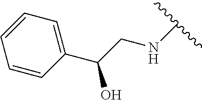 [SMILES: *NC[C@@H](O)c1ccccc1] | —H | —H |
| 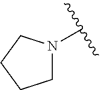 [SMILES: *N1CCCC1] | —H | —H |
| 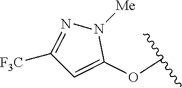 [SMILES: *Oc1cc(C(F)(F)F)nn1C] | —H | —H |
| 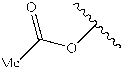 [SMILES: *OC(C)=O] | —H | —H |
| —Br | —CH₃ | —H |
| —Br | —CH₃ | —H |
| 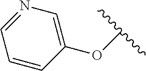 [SMILES: *Oc1cccnc1] | —CH₃ | —H |
| —Br | —H | —CH₃ |
| 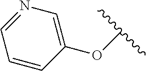 [SMILES: *Oc1cccnc1] | —H | —CH₃ |
| —Br | —H | —CF₃ |
| 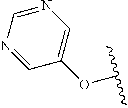 [SMILES: *Oc1cncnc1] | —H | —CF₃ |
| —Br | —H | —CH₂CH₃ |

In certain embodiments, the present invention provides a compound of Formula I, wherein LBM is a DCAF16 binding moiety thereby forming a compound of Formula I-k-2:

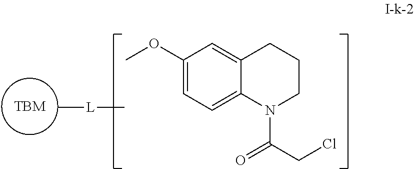

or a pharmaceutically acceptable salt thereof as described and defined in Zhang, X. et al., bioRxiv (doi: https://doi.org/10.1101/443804), the entirety of each of which is herein incorporated by reference, and wherein L and TBM are as defined above and described in embodiments herein.

In certain embodiments, the present invention provides a compound of Formula I, wherein LBM is a RNF114 binding moiety thereby forming a compound of Formula I-k-3:

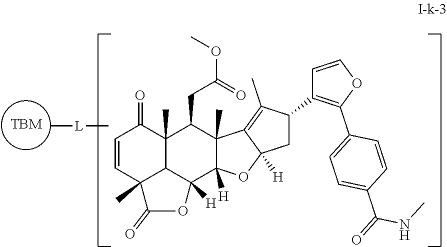

or a pharmaceutically acceptable salt thereof, as described and defined in Spradin, J. N. et al., bioRxiv (doi: https://doi.org/10.1101/436998), the entirety of each of which is herein incorporated by reference, and wherein L and TBM are as defined above and described in embodiments herein.

In certain embodiments, the present invention provides a compound of Formula I, wherein LBM is a RNF4 binding moiety thereby forming a compound of Formula I-k-4:

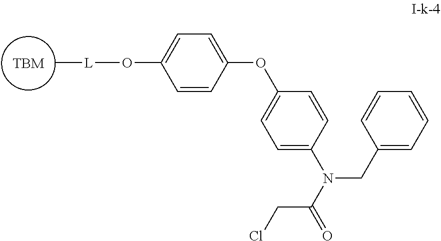

or a pharmaceutically acceptable salt thereof, as described and defined in Ward, C. C., et al., bioRxiv (doi: https://doi.org/10.1101/439125), the entirety of each of which is herein incorporated by reference, and wherein L and TBM are as defined above and described in embodiments herein.

In certain embodiments, the present invention provides a compound of Formula I, wherein LBM is a E3 ubiquitin ligase (cereblon) binding moiety thereby forming a compound of Formula I-l-1, I-l-2, I-l-3, or I-l-4:

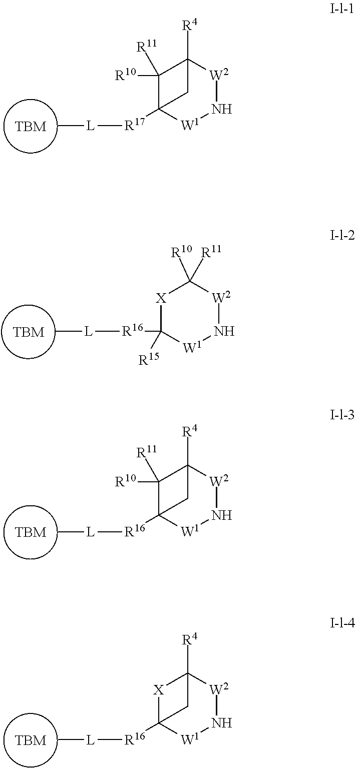

or a pharmaceutically acceptable salt thereof, wherein L and TBM are as defined above and described herein, and wherein each of the variables R⁴, R¹⁰, R¹¹, R¹⁵, R¹⁶, R¹⁷, W¹, W², and X is as defined in WO 2019/099868 which is herein incorporated by reference in its entirety, and wherein

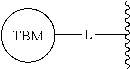

is attached to R¹⁷ or R¹⁶ at the site of attachment of R¹² as defined in WO 2018/237026, such that

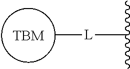

takes the place of the R¹² substituent.

In certain embodiments, the present invention provides a compound of Formula I, wherein LBM is a cereblon E3 ubiquitin ligase binding moiety, a DCAF15 E3 ubiquitin ligase binding moiety, or a VHL E3 ubiquitin ligase binding moiety; thereby forming a compound of Formula I-m-1, I-m-2, or I-m-3:

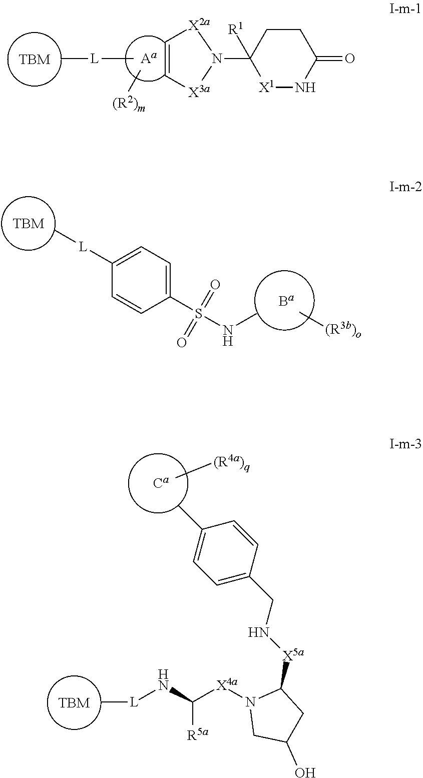

or a pharmaceutically acceptable salt thereof, wherein L and TBM is as defined above and described in embodiments herein, and wherein:
each of X¹, X^{2a}, and X^{3a} is independently a bivalent moiety selected from a covalent bond, —CH₂—, —C(O)—, —C(S)—, or

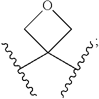 [SMILES: *C1(*)COC1]

each of X^{4a} and X^{5a} is independently a bivalent moiety selected from —CH₂—, —C(O)—, —C(S)—, or

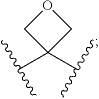 [SMILES: *C1(*)COC1]

R¹ is hydrogen, deuterium, halogen, —CN, —OR, —SR,
—S(O)R, —S(O)₂R, —NR₂, or an optionally substituted C₁₋₄ aliphatic;
each of R², R^{3b}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂,
—C(O)N(R)OR, —OC(O)R, —OC(O)NR₂, —N(R)C(O)OR, —N(R)C(O)R, —N(R)C(O)NR₂, or —N(R)S(O)₂R;
R^{5a} is hydrogen or C₁₋₆ aliphatic;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A^{a} is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen and sulfur, or 5-membered heteroaryl with 1-3 heteroatoms independently selected from nitrogen, oxygen and sulfur;
Ring B^{a} is selected from 6-membered aryl containing 0-2 nitrogen atoms or a 8-10 membered bicyclic heteroaryl having 1-5 heteroatoms independently selected from nitrogen, oxygen, and sulfur;
Ring C^{a} is a selected from 6-membered aryl containing 0-2 nitrogen atoms or a 5-membered heteroaryl with 1-3 heteroatoms independently selected from nitrogen, oxygen and sulfur;
m is 0, 1, 2, 3 or 4;
o is 0, 1, 2, 3 or 4;
q is 0, 1, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides a compound of Formula I-m-1, wherein LBM is an E3 ubiquitin ligase (cereblon) binding moiety thereby forming a compound of Formula I-m-4 or I-m-5:

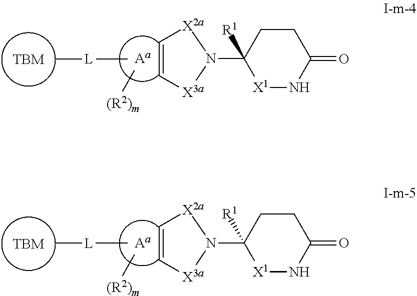

or a pharmaceutically acceptable salt thereof, wherein TBM, L, Ring A^{a}, X¹, X^{2a}, X^{3a}, R¹, R² and m are as described above.

As defined above and described herein, each of X¹, X^{2a}, and X^{3a} is independently a bivalent moiety selected from a covalent bond, —CH₂—, —C(O)—, —C(S)—, or

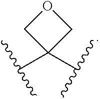 [SMILES: *C1(*)COC1]

In some embodiments, X¹ is a covalent bond, —CH₂—, —C(O)—, —C(S)—, or

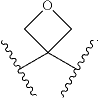 [SMILES: *C1(*)COC1]

In some embodiments, X¹ is selected from those depicted in Table 1, below.

In some embodiments, X^{2a} is a covalent bond, —CH₂—, —C(O)—, —C(S)—, or

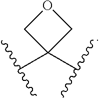 [SMILES: *C1(*)COC1]

In some embodiments, X^{2a} is selected from those depicted in Table 1, below.

In some embodiments, X^{3a} is a covalent bond, —CH₂—, —C(O)—, —C(S)—, or

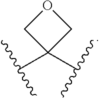 [SMILES: *C1(*)COC1]

In some embodiments, X^{3a} is selected from those depicted in Table 1, below.

As defined above and described herein, each of X^{4a} and X^{5a} is independently a bivalent moiety selected from —CH₂—, —C(O)—, —C(S)—, or

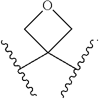 [SMILES: *C1(*)COC1]

In some embodiments, X^{4a} is —CH₂—, —C(O)—, —C(S)—, or

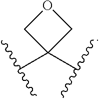 [SMILES: *C1(*)COC1]

In some embodiments, X^{4a} is selected from those depicted in Table 1, below.

In some embodiments, X^{5a} is —CH₂—, —C(O)—, —C(S)—, or

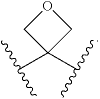 [SMILES: *C1(*)COC1]

In some embodiments, X^{5a} is selected from those depicted in Table 1, below.

As defined above and described herein, R¹ is hydrogen, deuterium, halogen, —CN, —OR, —SR, —S(O)R, —S(O)₂R, —NR₂, or an optionally substituted C₁₋₄ aliphatic.

In some embodiments, R¹ is hydrogen. In some embodiments, R¹ is deuterium. In some embodiments, R¹ is halogen. In some embodiments, R¹ is —CN. In some embodiments, R¹ is —OR. In some embodiments, R¹ is —SR. In some embodiments, R¹ is —S(O)R. In some embodiments, R¹ is —S(O)₂R. In some embodiments, R¹ is —NR₂. In some embodiments, R¹ is optionally substituted C₁₋₄ aliphatic.

In some embodiments, R¹ is selected from those depicted in Table 1, below.

As defined above and described herein, each of R², R^{3b}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R² is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R² is selected from those depicted in Table 1, below.

In some embodiments, R^{3b} is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R^{3b} is methyl.

In some embodiments, R^{3b} is selected from those depicted in Table 1, below.

In some embodiments, R^{4a} is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R^{4a} is methyl.

In some embodiments, R^{4a} is selected from those depicted in Table 1, below.

As defined above and described herein, R^{5a} is hydrogen or C₁₋₆ aliphatic.

In some embodiments, R^{5a} is t-butyl.

In some embodiments, R^{5a} is selected from those depicted in Table 1, below.

As defined above and described herein, each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁶ is an optionally substituted C₁₋₆ aliphatic group. In some embodiments, R⁶ is an optionally substituted phenyl. In some embodiments, R⁶ is an optionally substituted 4-7 membered saturated or partially unsaturated heterocyclic ring having 1-2 heteroatoms independently selected from nitrogen, oxygen, and sulfur. In some embodiments, R⁶ is an optionally substituted 5-6 membered heteroaryl ring having 1-4 heteroatoms independently selected from nitrogen, oxygen, and sulfur.

In some embodiments, R⁶ is selected from those depicted in Table 1, below.

As defined above and described herein, Ring A^{a} is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen and sulfur, or 5-membered heteroaryl with 1-3 heteroatoms independently selected from nitrogen, oxygen and sulfur.

In some embodiments Ring A^{a} is a fused 6-membered aryl containing 0-2 nitrogen atoms. In some embodiments Ring A^{a} is a fused 5 to 7-membered partially saturated carbocyclyl. In some embodiments Ring A^{a} is a fused 5 to 7-membered partially saturated heterocyclyl with 1-2 heteroatoms independently selected from nitrogen, oxygen and sulfur. In some embodiments Ring A^{a} is a fused 5-membered heteroaryl with 1-3 heteroatoms independently selected from nitrogen, oxygen and sulfur.

In some embodiments, Ring A^{a} is a fused phenyl.

In some embodiments, Ring A^{a} is selected from those depicted in Table 1, below.

As defined above and described herein, Ring B^{a} is selected from 6-membered aryl containing 0-2 nitrogen atoms or a 8-10 membered bicyclic heteroaryl having 1-5 heteroatoms independently selected from nitrogen, oxygen, and sulfur.

In some embodiments, Ring B^{a} is a 6-membered aryl containing 0-2 nitrogen atoms. In some embodiments, Ring B^{a} is a 8-10 membered bicyclic heteroaryl having 1-5 heteroatoms independently selected from nitrogen, oxygen, and sulfur.

In some embodiments, Ring B^{a} is

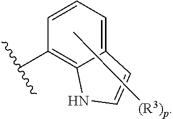

In some embodiments, Ring B^{a} is selected from those depicted in Table 1, below.

As defined above and described herein, Ring C^{a} is selected from 6-membered aryl containing 0-2 nitrogen atoms or a 5-membered heteroaryl with 1-3 heteroatoms independently selected from nitrogen, oxygen and sulfur.

In some embodiments, Ring C^{a} is a 6-membered aryl containing 0-2 nitrogen atoms. In some embodiments, Ring C^{a} is a 5-membered heteroaryl with 1-3 heteroatoms independently selected from nitrogen, oxygen and sulfur.

In some embodiments, Ring C^{a} is

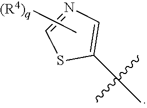

In some embodiments, Ring C^{a} is selected from those depicted in Table 1, below.

As defined above and described herein, m is 0, 1, 2, 3 or 4.

In some embodiments, m is 0. In some embodiments, m is 1. In some embodiments, m is 2. In some embodiments, m is 3. In some embodiments, m is 4.

In some embodiments, m is selected from those depicted in Table 1, below.

In some embodiments, o is selected from those depicted in Table 1, below.

As defined above and described herein, o is 0, 1, 2, 3 or 4.

In some embodiments, o is 0. In some embodiments, o is 1. In some embodiments, o is 2. In some embodiments, o is 3. In some embodiments, o is 4.

In some embodiments, o is selected from those depicted in Table 1, below.

As defined above and described herein, q is 0, 1, 2, 3 or 4.

In some embodiments, q is 0. In some embodiments, q is 1. In some embodiments, q is 2. In some embodiments, q is 3. In some embodiments, q is 4.

In some embodiments, q is selected from those depicted in Table 1, below.

As defined above and described herein, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is phenyl. In some embodiments, R is a 4-7 membered saturated or partially unsaturated heterocyclic having 1-2 heteroatoms independently selected from nitrogen, oxygen, and sulfur. In some embodiments, R is a 5-6 membered heteroaryl ring having 1-4 heteroatoms independently selected from nitrogen, oxygen, and sulfur. In some embodiments, two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is selected from those depicted in Table 1, below.

In certain embodiments, the present invention provides a compound of Formula I, wherein LBM is a VHL E3 ubiquitin ligase binding moiety, thereby forming a compound of Formula I-n:

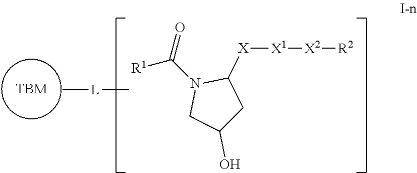

or a pharmaceutically acceptable salt thereof, wherein L and TBM is as defined above and described in embodiments herein, and wherein:
X is —C(O)—, —C(O)NR—, —SO₂—, —SO₂NR—, or an optionally substituted 5-membered heterocyclic ring;
X¹ is a bivalent group selected from a covalent bond, —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-6 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R)₂R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
R^{A} is an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R² is hydrogen, halogen, —CN,

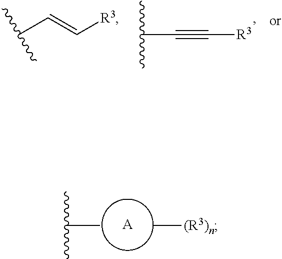

Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R³ is independently hydrogen, R^{A}, halogen, C₁₋₆ aliphatic, C₁₋₆ haloaliphatic, —CN, —NO₂, —OR, —SR, —N(R)₂, —Si(R)₃, —SO₂R,
—SO₂N(R)₂, —S(O)R, —C(O)R, —C(O)OR, —C(O)N(R)₂, —C(O)N(R)OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, or —N(R)SO₂R; or
two R³ groups are optionally taken together to form an optionally substituted 5-7 membered partially unsaturated or aryl fused ring having 0-2 heteroatoms independently selected from nitrogen, oxygen, and sulfur;
n is 0, 1, 2, 4, or 5.

As defined above and described herein, in some embodiments, X is —C(O)—, —C(O)NR—, —SO₂—, —SO₂NR—, or an optionally substituted 5-membered heterocyclic ring.

In some embodiments, X is —C(O)—. In some embodiments, X is —C(O)NR—. In some embodiments, X is —SO₂—. In some embodiments, X is —SO₂NR—. In some embodiments, X is an optionally substituted 5-membered heterocyclic ring.

In some embodiments, X is —C(O)NH—. In some embodiments, X is

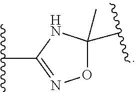 [SMILES: *C1=NOC(*)(C)N1]

In some embodiments, X is selected from those depicted in Table 1, below.

As defined above and described herein, in some embodiments, X¹ is a bivalent group selected from a covalent bond, —O—, —C(O)—, —C(S)—, —C(R)₂—, —NR—, —S(O)—, or —SO₂—.

In some embodiments, X¹ is a covalent bond. In some embodiments, X¹ is —O—. In some embodiments, X¹ is —C(O)—. In some embodiments, X¹ is —C(S)—. In some embodiments, X¹ is —C(R)₂—. In some embodiments, X¹ is —NR—. In some embodiments, X¹ is —S(O)—. In some embodiments, X¹ is —SO₂—.

In some embodiments, X¹ is

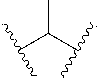 [SMILES: *C(*)C]

In some embodiments, X¹ is

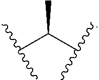

In some embodiments, X¹ is

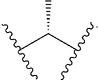

In some embodiments, X¹ is selected from those depicted in Table 1, below.

As defined above and described herein, in some embodiments, X² is an optionally substituted bivalent group selected from C₁₋₆ saturated or unsaturated alkylene, phenylenyl, a 5-6 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur.

In some embodiments, X² is an optionally substituted C₁₋₆ saturated or unsaturated alkylene. In some embodiments, X² is an optionally substituted phenylenyl. In some embodiments, X² is an optionally substituted 5-6 membered heteroarylenyl containing 1-4 heteroatoms independently selected from nitrogen, oxygen, and sulfur. In some embodiments, X² is an optionally substituted 4-11 membered saturated or partially unsaturated monocyclic, bicyclic, bridged bicyclic, or spirocyclic carbocyclylenyl. In some embodiments, X² is an optionally substituted 4-11 membered saturated or partially unsaturated monocyclic, bicyclic, bridged bicyclic, or spirocyclic heterocyclylenyl with 1-3 heteroatoms independently selected from nitrogen, oxygen, and sulfur.

In some embodiments, X² is selected from those depicted in Table 1, below.

As defined above and described herein, in some embodiments, R¹ is R^{z}, —C(R)₂R^{z}, —OR, —SR, —N(R)₂, —C(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R.

In some embodiments, R¹ is R^{z}. In some embodiments, R¹ is —C(R)₂R^{z}. In some embodiments, R¹ is —OR. In some embodiments, R¹ is —SR. In some embodiments, R¹ is —N(R)₂. In some embodiments, R¹ is —C(R)₂OR. In some embodiments, R¹ is —C(R)₂N(R)₂. In some embodiments, R¹ is —C(R)₂NRC(O)R. In some embodiments, R¹ is —C(R)₂NRC(O)N(R)₂. In some embodiments, R¹ is —NRC(O)OR. In some embodiments, R¹ is —NRC(O)R. In some embodiments, R¹ is —NRC(O)N(R)₂. In some embodiments, R¹ is —NRSO₂R.

In some embodiments, R¹ is

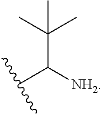 [SMILES: *C(N)C(C)(C)C]

In some embodiments, R¹ is

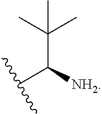 [SMILES: *[C@H](N)C(C)(C)C]

In some embodiments, R¹ is

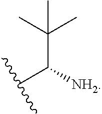 [SMILES: *[C@@H](N)C(C)(C)C]

In some embodiments, R¹ is

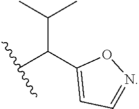 [SMILES: *C(c1ccno1)C(C)C]

In some embodiments, R¹ is

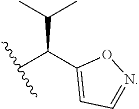 [SMILES: *[C@H](c1ccno1)C(C)C]

In some embodiments, R¹ is

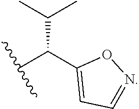 [SMILES: *[C@@H](c1ccno1)C(C)C]

In some embodiments, R¹ is

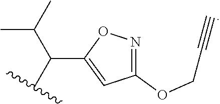 [SMILES: *C(c1cc(OCC#C)no1)C(C)C]

In some embodiments, R¹ is

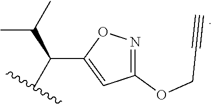 [SMILES: *[C@H](c1cc(OCC#C)no1)C(C)C]

In some embodiments, R¹ is

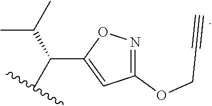 [SMILES: *[C@@H](c1cc(OCC#C)no1)C(C)C]

In some embodiments, R¹ is

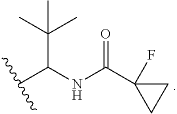 [SMILES: *C(NC(=O)C1(F)CC1)C(C)(C)C]

In some embodiments, R¹ is

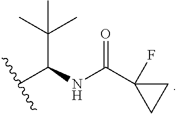 [SMILES: *[C@H](NC(=O)C1(F)CC1)C(C)(C)C]

In some embodiments, R¹ is

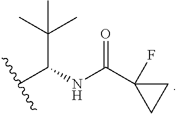 [SMILES: *[C@@H](NC(=O)C1(F)CC1)C(C)(C)C]

In some embodiments, R¹ is selected from those depicted in Table 1, below.

As defined above and described herein, in some embodiments, R² is hydrogen, halogen, —CN,

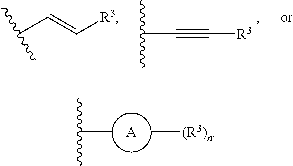

In some embodiments, R² is hydrogen. In some embodiments, R² is halogen. In some embodiments, R² is —CN. In some embodiments, R² is

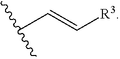

In some embodiments, R² is

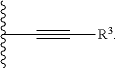

In some embodiments, R² is

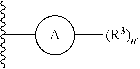

In some embodiments, R² is chloro. In some embodiments, R² is

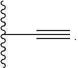 [SMILES: *C#C]

In some embodiments, R² is selected from those depicted in Table 1, below.

As defined above and described herein, in some embodiments, Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur.

In some embodiments, Ring A is phenyl. In some embodiments, Ring A is a 5-6 membered heteroaryl containing 1-4 heteroatoms independently selected from nitrogen, oxygen, and sulfur. In some embodiments, Ring A is 4 to 9-membered saturated or partially unsaturated monocyclic, bicyclic, bridged bicyclic, or spirocyclic carbocyclyl. In some embodiments, Ring A is a 4 to 9-membered saturated or partially unsaturated monocyclic, bicyclic, bridged bicyclic, or spirocyclic heterocyclyl with 1-3 heteroatoms independently selected from nitrogen, oxygen, and sulfur.

In some embodiments, Ring A is

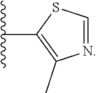 [SMILES: *c1scnc1C]

In some embodiments, Ring A is

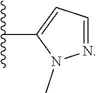 [SMILES: *c1ccnn1C]

In some embodiments, Ring A is selected from those depicted in Table 1, below.

As defined above and described herein, in some embodiments, each of R³ is independently hydrogen, R^{z}, halogen, —CN, —NO₂, —OR, —SR, —N(R)₂, —Si(R)₃, —SO₂R, —SO₂NR₂, —S(O)R, —C(O)R, —C(O)OR, —C(O)N(R)₂, —C(O)N(R)OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, or —N(R)SO₂R, or two R³ groups are optionally taken together to form an optionally substituted 5-7 membered partially unsaturated or aryl fused ring having 0-2 heteroatoms independently selected from nitrogen, oxygen, and sulfur.

In some embodiments, R³ is hydrogen. In some embodiments, R³ is R^{z}. In some embodiments, R³ is halogen. In some embodiments, R³ is —CN. In some embodiments, R³ is —NO₂. In some embodiments, R³ is —OR. In some embodiments, R³ is —SR. In some embodiments, R³ is —N(R)₂. In some embodiments, R³ is —Si(R)₃. In some embodiments, R³ is —SO₂R. In some embodiments, R³ is —SO₂NR₂. In some embodiments, R³ is —S(O)R. In some embodiments, R³ is —C(O)R. In some embodiments, R³ is —C(O)OR. In some embodiments, R³ is —C(O)N(R)₂. In some embodiments, R³ is —C(O)N(R)OR. In some embodiments, R³ is —C(R)₂NRC(O)R. In some embodiments, R³ is —C(R)₂NRC(O)N(R)₂. In some embodiments, R³ is —OC(O)R. In some embodiments, R³ is —OC(O)N(R)₂. In some embodiments, R³ is —OP(O)(R)₂. In some embodiments, R³ is —OP(O)(OR)₂. In some embodiments, R³ is —OP(O)(OR)N(R)₂. In some embodiments, R³ is —OP(O)(N(R)₂)₂—. In some embodiments, R³ is —N(R)C(O)OR. In some embodiments, R³ is —N(R)C(O)R. In some embodiments, R³ is —NRC(O)N(R)₂. In some embodiments, R³ is —N(R)SO₂R. In some embodiments, R³ is —NP(O)(R)₂. In some embodiments, R³ is —N(R)P(O)(OR)₂. In some embodiments, R³ is —N(R)P(O)(OR)N(R)₂. In some embodiments, R³ is —N(R)P(O)(N(R)₂)₂. In some embodiments, R³ is —N(R)SO₂R. In some embodiments, two R³ groups are optionally taken together to form an optionally substituted 5-7 membered partially unsaturated or aryl fused ring having 0-2 heteroatoms independently selected from nitrogen, oxygen, and sulfur.

In some embodiments, R³ is methyl.

In some embodiments, R³ is selected from those depicted in Table 1, below.

As defined above and described herein, in some embodiments, n is 0, 1, 2, 4, or 5.

In some embodiments, n is 0. In some embodiments, n is 1. In some embodiments, n is 2. In some embodiments, n is 3. In some embodiments, n is 4. In some embodiments, n is 5.

In some embodiments, n is selected from those depicted in Table 1, below.

In certain embodiments, the present invention provides a compound of Formula I-aa-1:

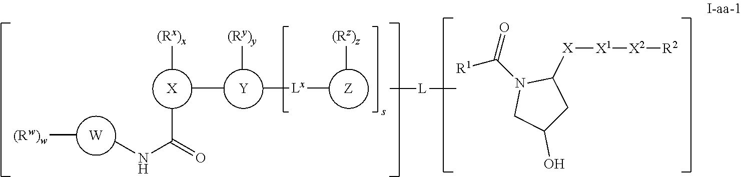

or a pharmaceutically acceptable salt, wherein:
Ring W and Ring Z are, independently, a ring selected from phenyl, naphthyl, a 5-10 membered heteroaryl containing 1-4 heteroatoms independently selected from nitrogen, oxygen, selenium, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring X is a bicyclic ring selected from a 9-11 membered partially unsaturated heterocyclylenyl with 1-4 heteroatoms independently selected from nitrogen, oxygen, and sulfur, and a 9-10 membered heteroarylenyl containing 1-4 heteroatoms independently selected from nitrogen, oxygen, and sulfur;
Ring Y is a bivalent ring selected from phenylenyl, and a 5-7 membered saturated or partially unsaturated carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
R^{w}, R^{x}, R^{y}, and R^{z} are, independently, hydrogen, R^{A}, halogen, —CN, —NO₂, —OR, —SR, —N(R)₂, —Si(R)₃, —S(O)₂R, —S(O)₂N(R)₂, —S(O)₂NRC(O)R, —S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NRS(O)₂R, —NP(O)(R)₂, —NRP(O)(OR)₂, —NRP(O)(OR)N(R)₂, —NRP(O)(N(R)₂)₂, or —NRS(O)₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L^{x} is a covalent bond or a bivalent, saturated or partially unsaturated, straight or branched C₁₋₅ hydrocarbon chain, wherein 0-3 methylene units of L^{x} are independently replaced by 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
s is 0 or 1; and
w, x, y, and z are, independently, 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(N(R)₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

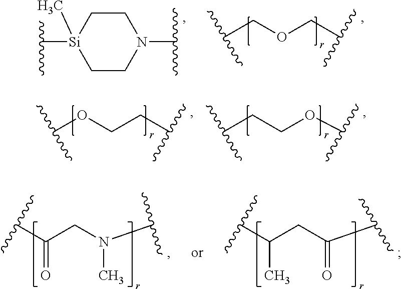

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
X is —C(O)—, —C(O)NR—, —SO₂—, —SO₂NR—, or an optionally substituted 5-membered heterocyclic ring;
X¹ is a bivalent group selected from a covalent bond, —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-6 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R)₂R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
R² is hydrogen, halogen, —CN,

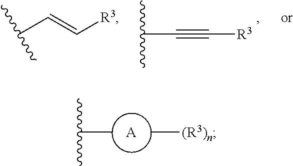

Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R³ is independently hydrogen, R^{A}, halogen, —CN, —NO₂, —OR, —SR, —N(R)₂, —Si(R)₃, —SO₂R, —SO₂N(R)₂, —S(O)R, —C(O)R, —C(O)OR, —C(O)N(R)₂, —C(O)N(R)OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, or —N(R)SO₂R; or
two R³ groups are optionally taken together to form an optionally substituted 5-7 membered partially unsaturated or aryl fused ring having 0-2 heteroatoms independently selected from nitrogen, oxygen, and sulfur;
n is 0, 1, 2, 4, or 5.

In certain embodiments, the present invention provides a compound of formula I-aa-1, wherein R¹ is

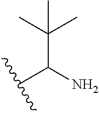 [SMILES: *C(N)C(C)(C)C]

(where one of the hydrogen atoms of the NH₂ group is replaced with -L-) as shown, to provide a compound of formula I-aa-2:

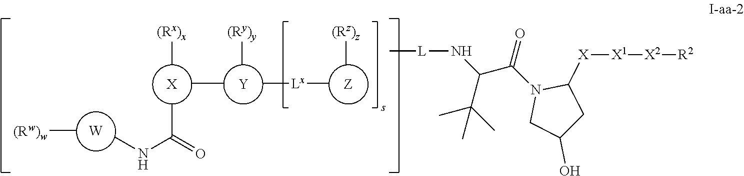

or a pharmaceutically acceptable salt thereof, wherein each of L, L^{x}, X, X¹, X², R², R^{w}, R^{x}, R^{y}, R^{z}, Ring W, Ring X, Ring Y, Ring Z, s, v, w, x, y, and z is as defined above and described in embodiments herein, both singly and in combination.

In some embodiments, LBM is

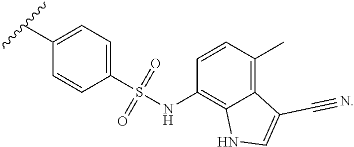 [SMILES: *c1ccc(S(=O)(=O)Nc2ccc(C)c3c(C#N)c[nH]c23)cc1]

In some embodiments, LBM is

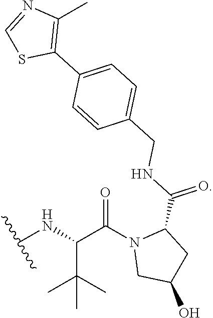 [SMILES: *N[C@H](C(=O)N1C[C@H](O)C[C@H]1C(=O)NCc1ccc(-c2scnc2C)cc1)C(C)(C)C]

In some embodiments, LBM is

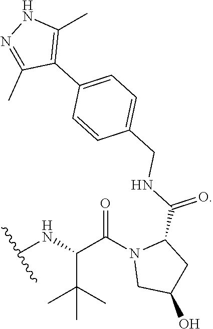 [SMILES: *N[C@H](C(=O)N1C[C@H](O)C[C@H]1C(=O)NCc1ccc(-c2c(C)n[nH]c2C)cc1)C(C)(C)C]

In some embodiments, LBM is

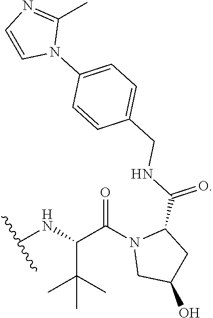 [SMILES: *N[C@H](C(=O)N1C[C@H](O)C[C@H]1C(=O)NCc1ccc(-n2ccnc2C)cc1)C(C)(C)C]

In some embodiments, LBM is

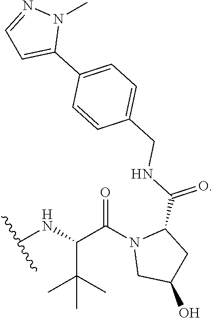 [SMILES: *N[C@H](C(=O)N1C[C@H](O)C[C@H]1C(=O)NCc1ccc(-c2ccnn2C)cc1)C(C)(C)C]

In some embodiments, LBM is

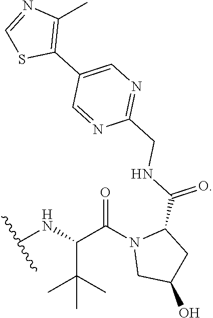 [SMILES: *N[C@H](C(=O)N1C[C@H](O)C[C@H]1C(=O)NCc1ncc(-c2scnc2C)cn1)C(C)(C)C]

In some embodiments, LBM is

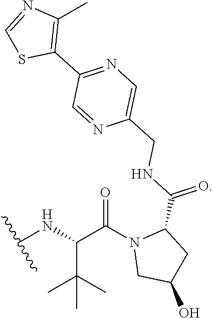 [SMILES: *N[C@H](C(=O)N1C[C@H](O)C[C@H]1C(=O)NCc1cnc(-c2scnc2C)cn1)C(C)(C)C]

In some embodiments, LBM is

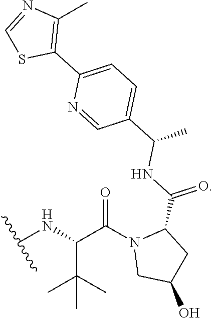 [SMILES: *N[C@H](C(=O)N1C[C@H](O)C[C@H]1C(=O)N[C@@H](C)c1ccc(-c2scnc2C)nc1)C(C)(C)C]

In some embodiments, LBM is

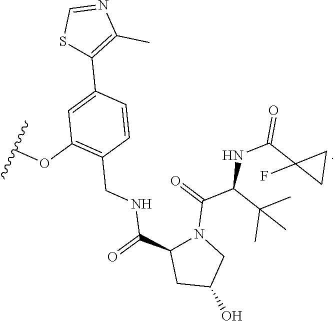 [SMILES: *Oc1cc(-c2scnc2C)ccc1CNC(=O)[C@@H]1C[C@@H](O)CN1C(=O)[C@@H](NC(=O)C1(F)CC1)C(C)(C)C]

In some embodiments, LBM is

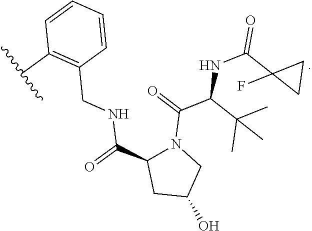 [SMILES: *c1ccccc1CNC(=O)[C@@H]1C[C@@H](O)CN1C(=O)[C@@H](NC(=O)C1(F)CC1)C(C)(C)C]

In some embodiments, LBM is

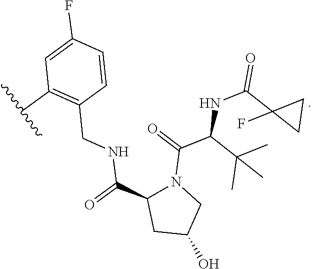 [SMILES: *c1cc(F)ccc1CNC(=O)[C@@H]1C[C@@H](O)CN1C(=O)[C@@H](NC(=O)C1(F)CC1)C(C)(C)C]

In some embodiments, LBM is

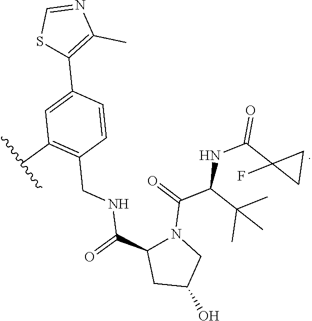 [SMILES: *c1cc(-c2scnc2C)ccc1CNC(=O)[C@@H]1C[C@@H](O)CN1C(=O)[C@@H](NC(=O)C1(F)CC1)C(C)(C)C]

In some embodiments, LBM is

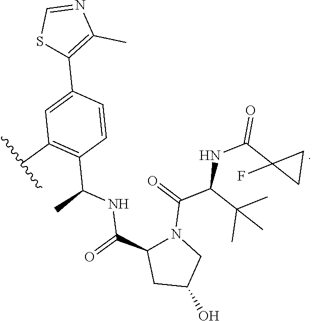 [SMILES: *c1cc(-c2scnc2C)ccc1[C@H](C)NC(=O)[C@@H]1C[C@@H](O)CN1C(=O)[C@@H](NC(=O)C1(F)CC1)C(C)(C)C]

In some embodiments, LBM is

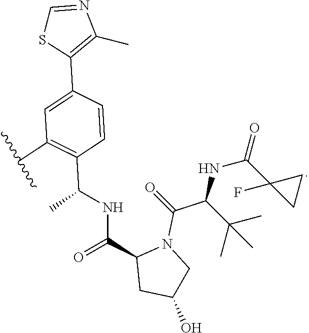 [SMILES: *c1cc(-c2scnc2C)ccc1[C@@H](C)NC(=O)[C@@H]1C[C@@H](O)CN1C(=O)[C@@H](NC(=O)C1(F)CC1)C(C)(C)C]

In some embodiments, LBM is

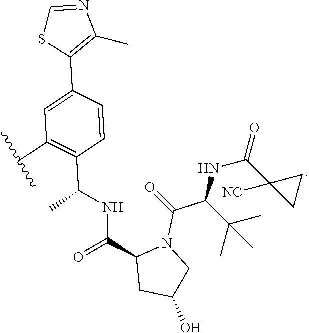 [SMILES: *c1cc(-c2scnc2C)ccc1[C@@H](C)NC(=O)[C@@H]1C[C@@H](O)CN1C(=O)[C@@H](NC(=O)C1(C#N)CC1)C(C)(C)C]

In some embodiments, LBM is

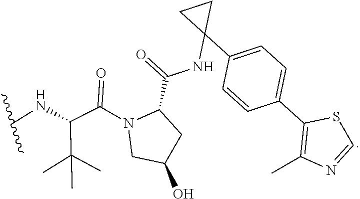 [SMILES: *N[C@H](C(=O)N1C[C@H](O)C[C@H]1C(=O)NC1(c2ccc(-c3scnc3C)cc2)CC1)C(C)(C)C]

In some embodiments, LBM is

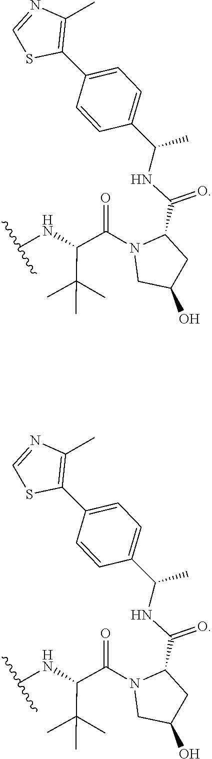 [SMILES: *N[C@H](C(=O)N1C[C@H](O)C[C@H]1C(=O)N[C@@H](C)c1ccc(-c2scnc2C)cc1)C(C)(C)C ; *N[C@H](C(=O)N1C[C@H](O)C[C@H]1C(=O)N[C@@H](C)c1ccc(-c2scnc2C)cc1)C(C)(C)C]

In some embodiments, LBM is

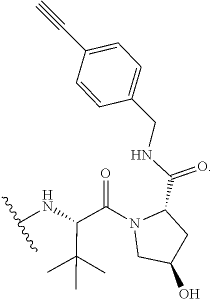 [SMILES: *N[C@H](C(=O)N1C[C@H](O)C[C@H]1C(=O)NCc1ccc(C#C)cc1)C(C)(C)C]

In some embodiments, LBM is

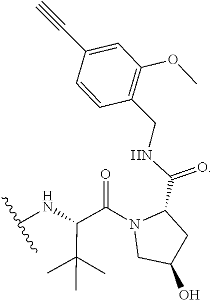 [SMILES: *N[C@H](C(=O)N1C[C@H](O)C[C@H]1C(=O)NCc1ccc(C#C)cc1OC)C(C)(C)C]

In some embodiments, LBM is

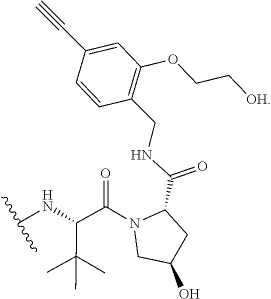 [SMILES: *N[C@H](C(=O)N1C[C@H](O)C[C@H]1C(=O)NCc1ccc(C#C)cc1OCCO)C(C)(C)C]

In some embodiments, LBM is

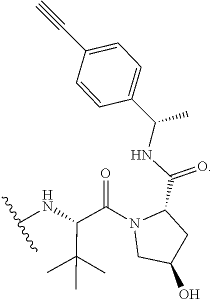 [SMILES: *N[C@H](C(=O)N1C[C@H](O)C[C@H]1C(=O)N[C@@H](C)c1ccc(C#C)cc1)C(C)(C)C]

In some embodiments, LBM is

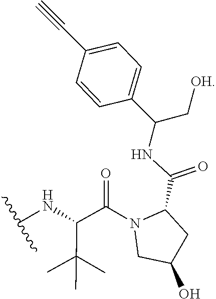 [SMILES: *N[C@H](C(=O)N1C[C@H](O)C[C@H]1C(=O)NC(CO)c1ccc(C#C)cc1)C(C)(C)C]

In some embodiments, LBM is

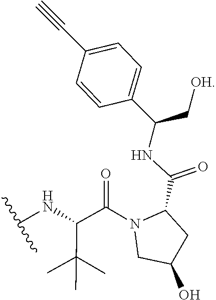 [SMILES: *N[C@H](C(=O)N1C[C@H](O)C[C@H]1C(=O)N[C@H](CO)c1ccc(C#C)cc1)C(C)(C)C]

In some embodiments, LBM is

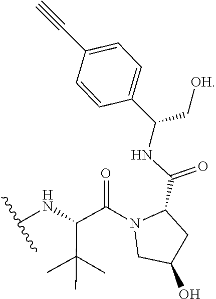 [SMILES: *N[C@H](C(=O)N1C[C@H](O)C[C@H]1C(=O)N[C@@H](CO)c1ccc(C#C)cc1)C(C)(C)C]

In some embodiments, LBM is

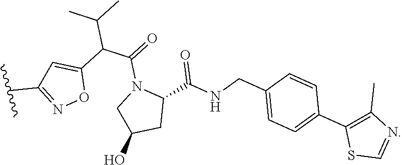 [SMILES: *c1cc(C(C(=O)N2C[C@H](O)C[C@H]2C(=O)NCc2ccc(-c3scnc3C)cc2)C(C)C)on1]

In some embodiments, LBM is

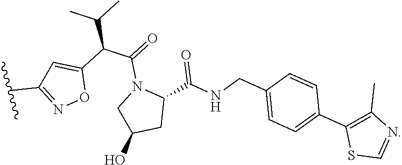 [SMILES: *c1cc([C@H](C(=O)N2C[C@H](O)C[C@H]2C(=O)NCc2ccc(-c3scnc3C)cc2)C(C)C)on1]

In some embodiments, LBM is

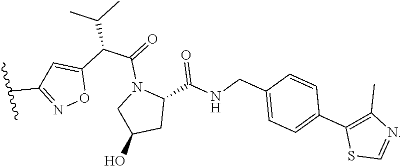 [SMILES: *c1cc([C@@H](C(=O)N2C[C@H](O)C[C@H]2C(=O)NCc2ccc(-c3scnc3C)cc2)C(C)C)on1]

In some embodiments, LBM is

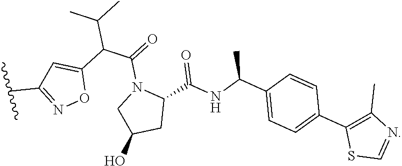 [SMILES: *c1cc(C(C(=O)N2C[C@H](O)C[C@H]2C(=O)N[C@@H](C)c2ccc(-c3scnc3C)cc2)C(C)C)on1]

In some embodiments, LBM is

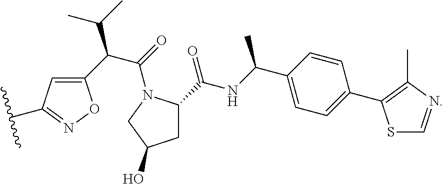 [SMILES: *c1cc([C@H](C(=O)N2C[C@H](O)C[C@H]2C(=O)N[C@@H](C)c2ccc(-c3scnc3C)cc2)C(C)C)on1]

In some embodiments, LBM is

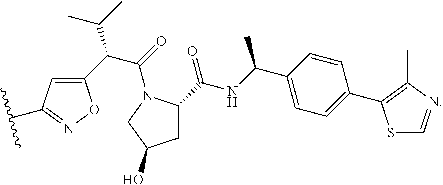 [SMILES: *c1cc([C@@H](C(=O)N2C[C@H](O)C[C@H]2C(=O)N[C@@H](C)c2ccc(-c3scnc3C)cc2)C(C)C)on1]

In some embodiments, LBM is

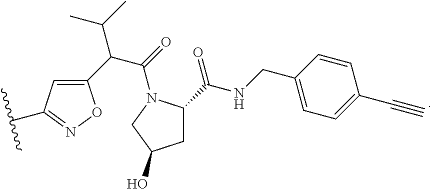 [SMILES: *c1cc(C(C(=O)N2C[C@H](O)C[C@H]2C(=O)NCc2ccc(C#C)cc2)C(C)C)on1]

In some embodiments, LBM is

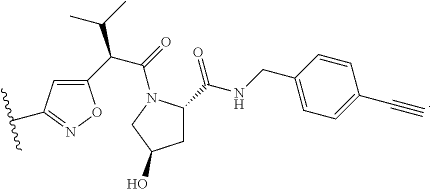 [SMILES: *c1cc([C@H](C(=O)N2C[C@H](O)C[C@H]2C(=O)NCc2ccc(C#C)cc2)C(C)C)on1]

In some embodiments, LBM is

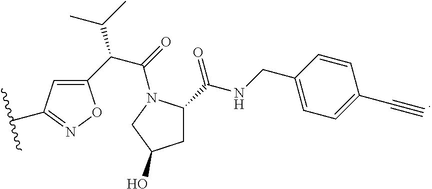 [SMILES: *c1cc([C@@H](C(=O)N2C[C@H](O)C[C@H]2C(=O)NCc2ccc(C#C)cc2)C(C)C)on1]

In some embodiments, LBM is

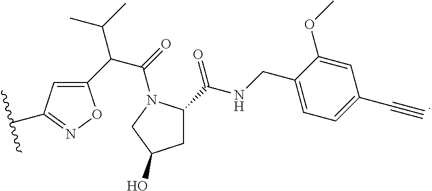 [SMILES: *c1cc(C(C(=O)N2C[C@H](O)C[C@H]2C(=O)NCc2ccc(C#C)cc2OC)C(C)C)on1]

In some embodiments, LBM is

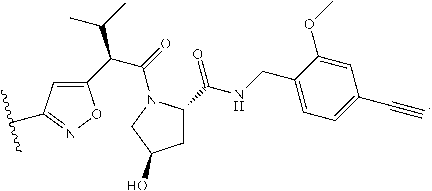 [SMILES: *c1cc([C@H](C(=O)N2C[C@H](O)C[C@H]2C(=O)NCc2ccc(C#C)cc2OC)C(C)C)on1]

In some embodiments, LBM is

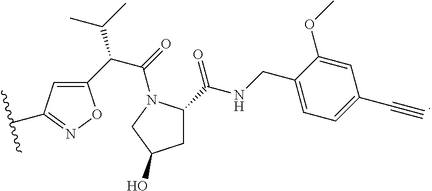 [SMILES: *c1cc([C@@H](C(=O)N2C[C@H](O)C[C@H]2C(=O)NCc2ccc(C#C)cc2OC)C(C)C)on1]

In some embodiments, LBM is

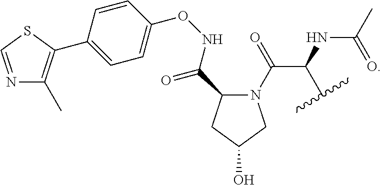 [SMILES: *[C@H](NC(C)=O)C(=O)N1C[C@H](O)C[C@H]1C(=O)NOc1ccc(-c2scnc2C)cc1]

In some embodiments, LBM is

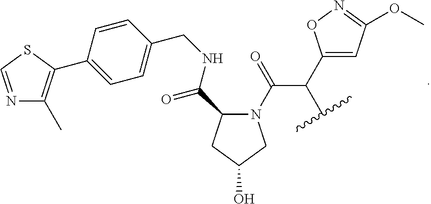 [SMILES: *C(C(=O)N1C[C@H](O)C[C@H]1C(=O)NCc1ccc(-c2scnc2C)cc1)c1cc(OC)no1]

In some embodiments, LBM is

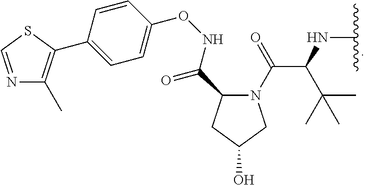 [SMILES: *N[C@H](C(=O)N1C[C@H](O)C[C@H]1C(=O)NOc1ccc(-c2scnc2C)cc1)C(C)(C)C]

In some embodiments, LBM is

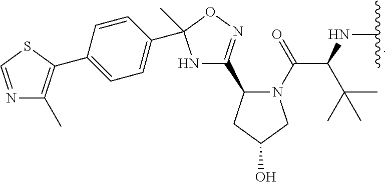 [SMILES: *N[C@H](C(=O)N1C[C@H](O)C[C@H]1C1=NOC(C)(c2ccc(-c3scnc3C)cc2)N1)C(C)(C)C]

In some embodiments, LBM is

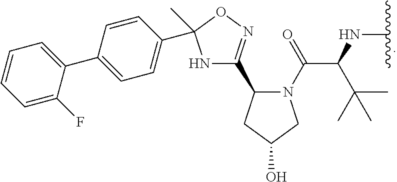 [SMILES: *N[C@H](C(=O)N1C[C@H](O)C[C@H]1C1=NOC(C)(c2ccc(-c3ccccc3F)cc2)N1)C(C)(C)C]

In some embodiments, LBM is

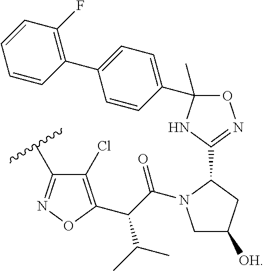 [SMILES: *c1noc([C@H](C(=O)N2C[C@H](O)C[C@H]2C2=NOC(C)(c3ccc(-c4ccccc4F)cc3)N2)C(C)C)c1Cl]

In some embodiments, LBM is

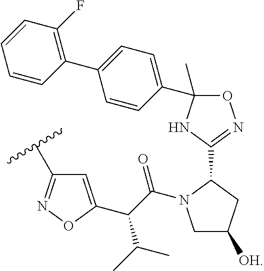 [SMILES: *c1cc([C@H](C(=O)N2C[C@H](O)C[C@H]2C2=NOC(C)(c3ccc(-c4ccccc4F)cc3)N2)C(C)C)on1]

In some embodiments, LBM is

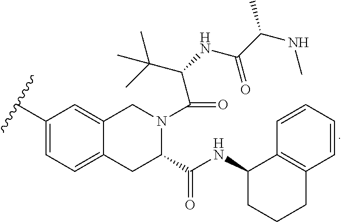 [SMILES: *c1ccc2c(c1)CN(C(=O)[C@@H](NC(=O)[C@H](C)NC)C(C)(C)C)[C@H](C(=O)N[C@@H]1CCCc3ccccc31)C2]

In some embodiments, LBM is

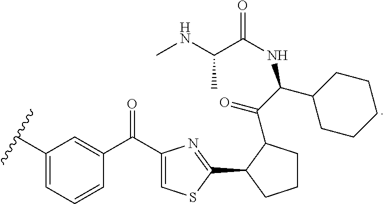 [SMILES: *c1cccc(C(=O)c2csc([C@@H]3CCCC3C(=O)[C@@H](NC(=O)[C@H](C)NC)C3CCCCC3)n2)c1]

In some embodiments, LBM is

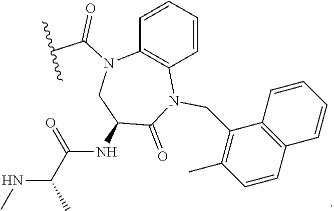 [SMILES: *C(=O)N1C[C@H](NC(=O)[C@H](C)NC)C(=O)N(Cc2c(C)ccc3ccccc23)c2ccccc21]

In some embodiments, LBM is

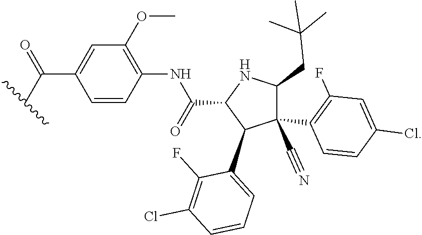 [SMILES: *C(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1]

In some embodiments, LBM is

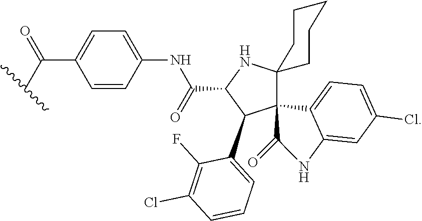 [SMILES: *C(=O)c1ccc(NC(=O)[C@@H]2NC3(CCCCC3)[C@@]3(C(=O)Nc4cc(Cl)ccc43)[C@H]2c2cccc(Cl)c2F)cc1]

In some embodiments, LBM is

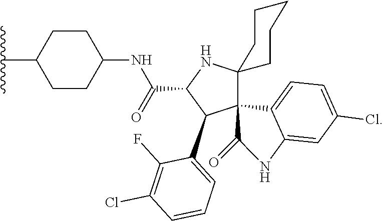 [SMILES: *C1CCC(NC(=O)[C@@H]2NC3(CCCCC3)[C@@]3(C(=O)Nc4cc(Cl)ccc43)[C@H]2c2cccc(Cl)c2F)CC1]

In some embodiments, LBM is

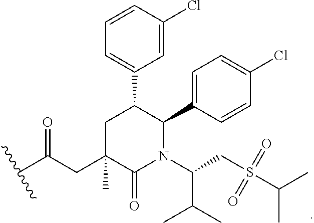 [SMILES: *C(=O)C[C@@]1(C)C[C@H](c2cccc(Cl)c2)[C@@H](c2ccc(Cl)cc2)N([C@H](CS(=O)(=O)C(C)C)C(C)C)C1=O]

In some embodiments, LBM is

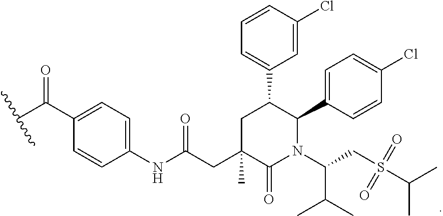 [SMILES: *C(=O)c1ccc(NC(=O)C[C@@]2(C)C[C@H](c3cccc(Cl)c3)[C@@H](c3ccc(Cl)cc3)N([C@H](CS(=O)(=O)C(C)C)C(C)C)C2=O)cc1]

In some embodiments, LBM is

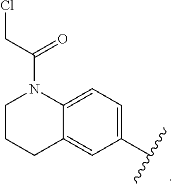 [SMILES: *c1ccc2c(c1)CCCN2C(=O)CCl]

In some embodiments, LBM is

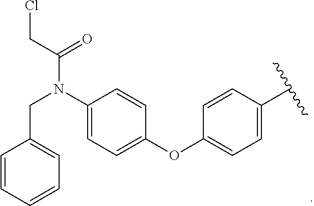 [SMILES: *c1ccc(Oc2ccc(N(Cc3ccccc3)C(=O)CCl)cc2)cc1]

In some embodiments, LBM is

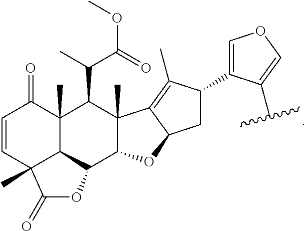 [SMILES: *c1cocc1[C@@H]1C[C@H]2O[C@@H]3[C@@H]4OC(=O)[C@]5(C)C=CC(=O)[C@](C)([C@@H](C(C)C(=O)OC)[C@]3(C)C2=C1C)[C@@H]45]

In some embodiments, LBM is

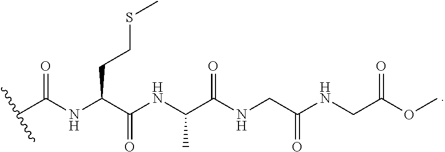 [SMILES: *C(=O)N[C@@H](CCSC)C(=O)N[C@@H](C)C(=O)NCC(=O)NCC(=O)OC]

In some embodiments, LBM is

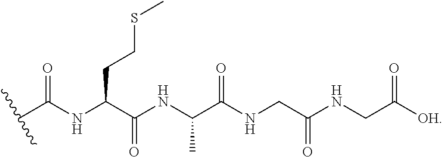 [SMILES: *C(=O)N[C@@H](CCSC)C(=O)N[C@@H](C)C(=O)NCC(=O)NCC(=O)O]

In some embodiments, LBM is

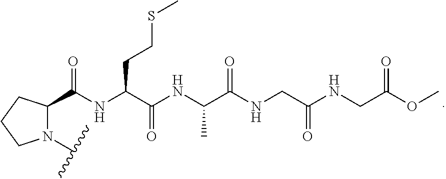 [SMILES: *N1CCC[C@H]1C(=O)N[C@@H](CCSC)C(=O)N[C@@H](C)C(=O)NCC(=O)NCC(=O)OC]

In some embodiments, LBM is

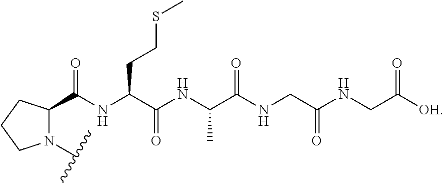 [SMILES: *N1CCC[C@H]1C(=O)N[C@@H](CCSC)C(=O)N[C@@H](C)C(=O)NCC(=O)NCC(=O)O]

In some embodiments, LBM is

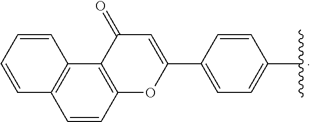 [SMILES: *c1ccc(-c2cc(=O)c3c(ccc4ccccc43)o2)cc1]

In some embodiments, LBM is

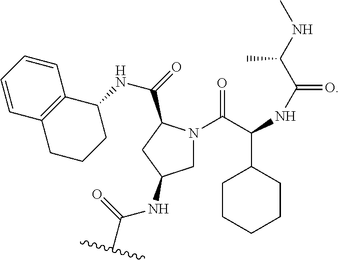 [SMILES: *C(=O)N[C@H]1C[C@@H](C(=O)N[C@@H]2CCCc3ccccc32)N(C(=O)[C@@H](NC(=O)[C@H](C)NC)C2CCCCC2)C1]

In some embodiments, LBM is

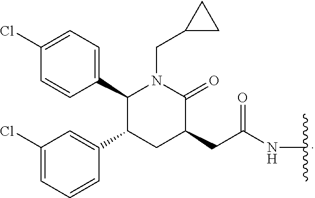 [SMILES: *NC(=O)C[C@H]1C[C@H](c2cccc(Cl)c2)[C@@H](c2ccc(Cl)cc2)N(CC2CC2)C1=O]

In some embodiments, LBM is

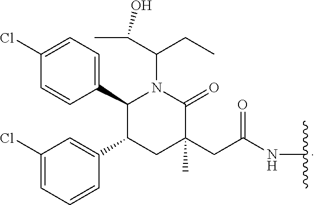 [SMILES: *NC(=O)C[C@@]1(C)C[C@H](c2cccc(Cl)c2)[C@@H](c2ccc(Cl)cc2)N(C(CC)[C@H](C)O)C1=O]

In some embodiments, LBM is

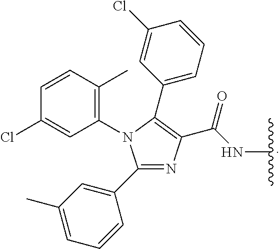 [SMILES: *NC(=O)c1nc(-c2cccc(C)c2)n(-c2cc(Cl)ccc2C)c1-c1cccc(Cl)c1]

In some embodiments, LBM is

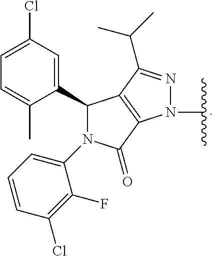 [SMILES: *n1nc(C(C)C)c2c1C(=O)N(c1cccc(Cl)c1F)[C@H]2c1cc(Cl)ccc1C]

In some embodiments, LBM is

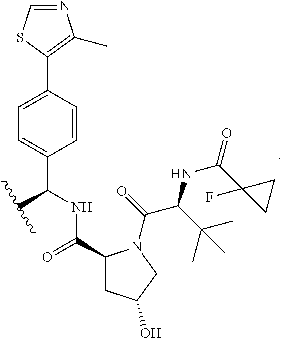 [SMILES: *[C@H](NC(=O)[C@@H]1C[C@@H](O)CN1C(=O)[C@@H](NC(=O)C1(F)CC1)C(C)(C)C)c1ccc(-c2scnc2C)cc1]

In certain embodiments, the present invention provides a compound of Formula I, wherein LBM is a CRBN E3 ubiquitin ligase binding moiety thereby forming a compound of Formula I-ll:

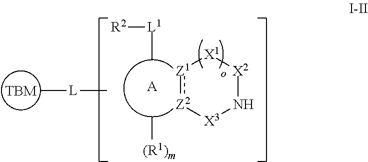

or a pharmaceutically acceptable salt thereof, wherein L and TBM are as defined above and described in embodiments herein, wherein:
each X¹ is independently —CH₂—, —O—, —NR—, —CF₂—,

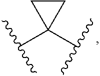 [SMILES: *C1(*)CC1]

### —C(O)—, —C(S)—, or

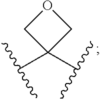 [SMILES: *C1(*)COC1]

X² and X³ are independently —CH₂—, —C(O)—, —C(S)—, or

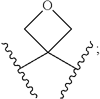 [SMILES: *C1(*)COC1]

Z¹ and Z² are independently a carbon atom or a nitrogen atom;
Ring A is a fused ring selected from benzo, a 4-6 membered saturated or partially unsaturated carbocyclic or heterocyclic ring having 1-3 heteroatoms independently selected from nitrogen, oxygen, and sulfur, and a 5-6 membered heteroaryl ring having 1-4 heteroatoms independently selected from nitrogen, oxygen, and sulfur;
L is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—;
each R¹ is independently selected from hydrogen, deuterium, R⁴, halogen, —CN, —NO₂, —OR, —SR, —NR₂, —S(O)₂R, —S(O)₂NR₂, —S(O)R, —CF₂R, —CR₂F, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃; or
two R¹ groups are optionally taken together to form an optionally substituted 5-8 membered partially unsaturated or aryl fused ring having 0-2 heteroatoms independently selected from nitrogen, oxygen, and sulfur;
each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur;
R² is selected from

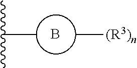

or hydrogen;
Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups;
each R³ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃;
each R⁴ is independently selected from an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
[symbol] is a single or double bond;
m is 0, 1, 2, 3 or 4;
n is 0, 1, 2, 3 or 4; and
o is 0, 1, or 2.

As defined above and described herein X¹ is a covalent bond, —CH₂—, —O—, —NR—, —CF₂—,

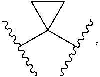 [SMILES: *C1(*)CC1]

### —C(O)—, —C(S)—, or

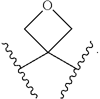 [SMILES: *C1(*)COC1]

In some embodiments, X¹ is a covalent bond. In some embodiments, X¹ is —CH₂—. In some embodiments, X¹ is —O—. In some embodiments, X¹ is —NR—. In some embodiments, X¹ is —CF₂—. In some embodiments, X¹ is

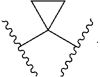 [SMILES: *C1(*)CC1]

In some embodiments, X¹ is —C(O)—. In some embodiments, X¹ is —C(S)—. In some embodiments, X¹ is

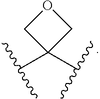 [SMILES: *C1(*)COC1]

In certain embodiments, X¹ is selected from those shown in the compounds of Table 1.

As defined above and described herein, X² and X³ are independently —CH₂—, —C(O)—, —C(S)—, or

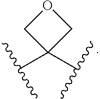 [SMILES: *C1(*)COC1]

In some embodiments, X² and X³ are independently —CH₂—. In some embodiments, X² and X³ are independently —C(O)—. In some embodiments, X² and X³ are independently —C(S)—. In some embodiments, X² and X³ are independently

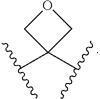 [SMILES: *C1(*)COC1]

In certain embodiments, X² and X³ are independently selected from those shown in the compounds of Table 1.

As defined above and described herein, X⁴ is a covalent bond, —CH₂—, —CR₂—, —O—, —NR—, —CF₂—,

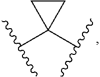 [SMILES: *C1(*)CC1]

### —C(O)—, —C(S)—, or

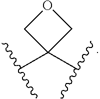 [SMILES: *C1(*)COC1]

In some embodiments, X⁴ is a covalent bond. In some embodiments, X⁴ is —CH₂—. In some embodiments, X⁴ is —CR₂—. In some embodiments, X⁴ is —O—. In some embodiments, X⁴ is —NR—. In some embodiments, X⁴ is —CF₂—. In some embodiments, X⁴ is

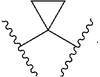 [SMILES: *C1(*)CC1]

In some embodiments, X⁴ is —C(O)—. In some embodiments, X⁴ is —C(S)—. In some embodiments, X⁴ is

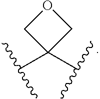 [SMILES: *C1(*)COC1]

In certain embodiments, X⁴ is selected from those shown in the compounds of Table 1.

As define above and described herein, Z¹ and Z² are independently a carbon atom or a nitrogen atom.

In some embodiments, Z¹ and Z² are independently a carbon atom. In some embodiments, Z¹ and Z² are independently a carbon atom.

In certain embodiments, Z¹ and Z² are independently selected from those shown in the compounds of Table 1.

As defined above and described herein, Ring A is fused ring selected from benzo or a 5-6 membered heteroaryl ring having 1-4 heteroatoms independently selected from nitrogen, oxygen, and sulfur.

In some embodiments, Ring A is benzo. In some embodiments, Ring A is a 5-6 membered heteroaryl ring having 1-4 heteroatoms independently selected from nitrogen, oxygen, and sulfur.

In some embodiments, Ring A is

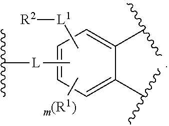

In certain embodiments, Ring A is selected from those shown in the compounds of Table 1.

In some embodiments, Ring C is a spiro-fused ring selected from a 4-10 membered saturated or partially unsaturated mono- or bicyclic carbocyclic or heterocyclic ring having 1-3 heteroatoms independently selected from nitrogen, oxygen, and sulfur. In some embodiments, Ring C is optionally further substituted with 1-2 oxo groups.

In certain embodiments, Ring C is selected from those shown in the compounds of Table 1.

As defined above and described herein, L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—.

In some embodiments, L¹ is a covalent bond. In some embodiments, L¹ is a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—.

In some embodiments, L¹ is —C(O)—.

In certain embodiments, L¹ is selected from those shown in the compounds of Table 1.

As defined above and described herein, each R¹ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃, or two R¹ groups are optionally taken together to form an optionally substituted 5-8 membered partially unsaturated or aryl fused ring having 0-2 heteroatoms independently selected from nitrogen, oxygen, and sulfur.

In some embodiments, R¹ is hydrogen. In some embodiments, R¹ is deuterium. In some embodiments, R¹ is R⁴. In some embodiments, R¹ is halogen. In some embodiments, R¹ is —CN. In some embodiments, R¹ is —NO₂. In some embodiments, R¹ is —OR. In some embodiments, R¹ is —SR. In some embodiments, R¹ is —NR₂. In some embodiments, R¹ is —S(O)₂R. In some embodiments, R¹ is —S(O)₂NR₂. In some embodiments, R¹ is —S(O)R. In some embodiments, R¹ is —CF₂R. In some embodiments, R¹ is —CF₃. In some embodiments, R¹ is —CR₂(OR). In some embodiments, R¹ is —CR₂(NR₂). In some embodiments, R¹ is —C(O)R. In some embodiments, R¹ is —C(O)OR. In some embodiments, R¹ is —C(O)NR₂. In some embodiments, R¹ is —C(O)N(R)OR. In some embodiments, R¹ is —OC(O)R. In some embodiments, R¹ is —OC(O)NR₂. In some embodiments, R¹ is —C(S)NR₂. In some embodiments, R¹ is —N(R)C(O)OR. In some embodiments, R¹ is —N(R)C(O)R. In some embodiments, R¹ is —N(R)C(O)NR₂. In some embodiments, R¹ is —N(R)S(O)₂R. In some embodiments, R¹ is —OP(O)R₂. In some embodiments, R¹ is —OP(O)(OR)₂. In some embodiments, R¹ is —OP(O)(OR)NR₂. In some embodiments, R¹ is —OP(O)(NR₂)₂. In some embodiments, R¹ is —Si(OR)R₂. In some embodiments, R¹ is —SiR₃. In some embodiments, two R¹ groups are optionally taken together to form an optionally substituted 5-8 membered partially unsaturated or aryl fused ring having 0-2 heteroatoms independently selected from nitrogen, oxygen, and sulfur.

In some embodiments, R¹ is fluoro. In some embodiments, R¹ is

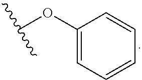 [SMILES: *Oc1ccccc1]

In certain embodiments, each R¹ is independently selected from those shown in the compounds of Table 1.

As defined above and described here, each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 4-7 membered saturated or partially unsaturated heterocyclic having 1-2 heteroatoms independently selected from nitrogen, oxygen, and sulfur. In some embodiments, R is an optionally substituted a 5-6 membered heteroaryl ring having 1-4 heteroatoms independently selected from nitrogen, oxygen, and sulfur. In some embodiments, two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

As defined above and described herein, R² is selected from

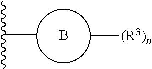

or hydrogen.

In some embodiment R² is

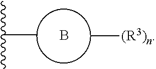

In some embodiments, R² is hydrogen.

In certain embodiments, R² is selected from those shown in the compounds of Table 1.

As defined above and described herein, Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups.

In some embodiments, Ring B is phenyl. In some embodiments, Ring B is a 4-10 membered saturated or partially unsaturated mono- or bicyclic carbocyclic or heterocyclic ring having 1-3 heteroatoms independently selected from nitrogen, oxygen, and sulfur In some embodiments, Ring B is a 5-6 membered heteroaryl ring having 1-4 heteroatoms independently selected from nitrogen, oxygen, and sulfur. In some embodiments, Ring B is further optionally substituted with 1-2 oxo groups.

In certain embodiments, Ring B is selected from those shown in the compounds of Table 1.

As defined above and described herein, each R³ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃.

In some embodiments, R³ is hydrogen. In some embodiments, R³ is deuterium. In some embodiments, R³ is R⁴. In some embodiments, R³ is halogen. In some embodiments, R³ is —CN. In some embodiments, R³ is —NO₂. In some embodiments, R³ is —OR. In some embodiments, R³ is —SR. In some embodiments, R³ is —NR₂. In some embodiments, R³ is —S(O)₂R. In some embodiments, R³ is —S(O)₂NR₂. In some embodiments, R³ is —S(O)R. In some embodiments, R³ is —CF₂R. In some embodiments, R³ is —CF₃. In some embodiments, R³ is —CR₂(OR). In some embodiments, R³ is —CR₂(NR₂). In some embodiments, R³ is —C(O)R. In some embodiments, R³ is —C(O)OR. In some embodiments, R³ is —C(O)NR₂. In some embodiments, R³ is —C(O)N(R)OR. In some embodiments, R³ is —OC(O)R. In some embodiments, R³ is —OC(O)NR₂. In some embodiments, R³ is —N(R)C(O)OR. In some embodiments, R³ is —N(R)C(O)R. In some embodiments, R³ is —N(R)C(O)NR₂. In some embodiments, R³ is —N(R)S(O)₂R. In some embodiments, R³ is —OP(O)R₂. In some embodiments, R³ is —OP(O)(OR)₂. In some embodiments, R³ is —OP(O)(OR)NR₂. In some embodiments, R³ is —OP(O)(NR₂)₂. In some embodiments, R³ is —SiR₃.

In certain embodiments, R³ is selected from those shown in the compounds of Table 1.

As defined above and described herein, each R⁴ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁴ is an optionally substituted C₁₋₆ aliphatic. In some embodiments, R⁴ is an optionally substituted phenyl. In some embodiments, R⁴ is an optionally substituted 4-7 membered saturated or partially unsaturated heterocyclic ring having 1-2 heteroatoms independently selected from nitrogen, oxygen, and sulfur. In some embodiments, R⁴ is an optionally substituted 5-6 membered heteroaryl ring having 1-4 heteroatoms independently selected from nitrogen, oxygen, and sulfur.

In certain embodiments, R⁴ is selected from those shown in the compounds of Table 1.

As defined above and described herein, [symbol] is a single or double bond.

In some embodiments, [symbol] is a single bond. In some embodiments, [symbol] is a double bond.

In certain embodiments, [symbol] is selected from those shown in the compounds of Table 1.

As defined above and described herein, m is 0, 1, 2, 3 or 4.

In some embodiments, m is 0. In some embodiments, m is 1. In some embodiments, m is 2. In some embodiments, m is 3. In some embodiments, m is 4.

In certain embodiments, m is selected from those shown in the compounds of Table 1.

As defined above and described herein, n is 0, 1, 2, 3 or 4.

In some embodiments, n is 0. In some embodiments, n is 1. In some embodiments, n is 2. In some embodiments, n is 3. In some embodiments, n is 4.

In certain embodiments, n is selected from those shown in the compounds of Table 1.

As defined above and described herein, o is 0, 1, or 2.

In some embodiments, n is 0. In some embodiments, n is 1. In some embodiments, m is 2.

In certain embodiments, o is selected from those shown in the compounds of Table 1.

In some embodiments, the present invention provides a compound of Formula I-cc, wherein Ring A is benzo, o is 1, X¹ is —CH₂—, X² and X³ are —C(O)—, and Z¹ and Z² are carbon atoms as shown, to provide a compound of Formula I-cc-1:

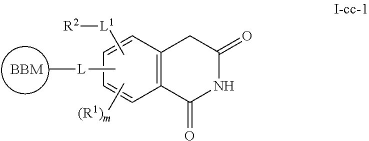

or a pharmaceutically acceptable salt thereof, wherein each of TBM, L, L¹, R¹, R², and m is as defined above and described in embodiments herein, both singly and in combination.

In some embodiments, the present invention provides a compound of Formula I-cc, wherein Ring A is benzo, o is 1, X¹, X² and X³ are —C(O)—, and Z¹ and Z² are carbon atoms as shown, to provide a compound of Formula I-cc-12:

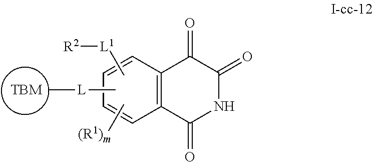

or a pharmaceutically acceptable salt thereof, wherein each of TBM, L, L¹, R¹, R², and m is as defined above and described in embodiments herein, both singly and in combination.

In some embodiments, LBM is

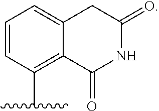 [SMILES: *c1cccc2c1C(=O)NC(=O)C2]

In some embodiments, LBM is

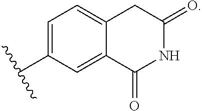 [SMILES: *c1ccc2c(c1)C(=O)NC(=O)C2]

In some embodiments, LBM is

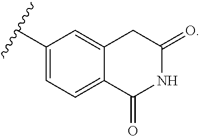 [SMILES: *c1ccc2c(c1)CC(=O)NC2=O]

In some embodiments, LBM is

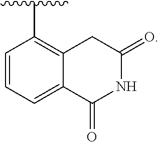 [SMILES: *c1cccc2c1CC(=O)NC2=O]

In some embodiments, LBM is

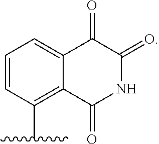 [SMILES: *c1cccc2c1C(=O)NC(=O)C2=O]

In some embodiments, LBM is

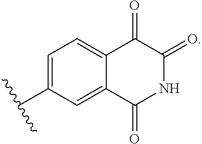 [SMILES: *c1ccc2c(c1)C(=O)NC(=O)C2=O]

In some embodiments, LBM is

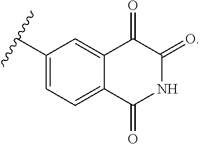 [SMILES: *c1ccc2c(c1)C(=O)C(=O)NC2=O]

In some embodiments, LBM is

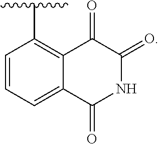 [SMILES: *c1cccc2c1C(=O)C(=O)NC2=O]

In some embodiments, LBM is selected from those in Table 1.

In certain embodiments, the present invention provides a compound of Formula I, wherein LBM is a RPN13 binding moiety thereby forming a compound of Formula I-o-1:

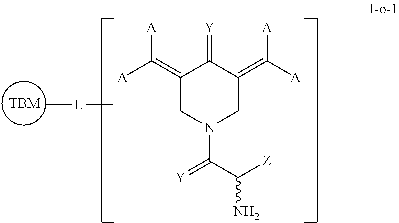

or a pharmaceutically acceptable salt thereof, wherein L and TBM are as defined above and described in embodiments herein, and wherein each of the variables A, Y, and Z is as described and defined in WO 2019/165229, the entirety of each of which is herein incorporated by reference.

In certain embodiments, the present invention provides a compound of Formula I, wherein LBM is a Ubr1 binding moiety as described in Shanmugasundaram, K. et al, J. Bio. Chem. 2019, doi: 10.1074/jbc.AC119.010790, the entirety of each of which is herein incorporated by reference, thereby forming a compound of Formula I-o-2 or I-o-3:

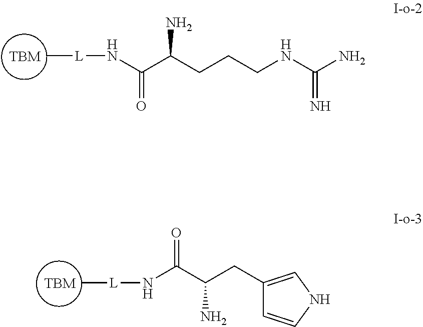

or a pharmaceutically acceptable salt thereof, wherein L and TBM are as defined above and described in embodiments herein.

In certain embodiments, the present invention provides a compound of Formula I, wherein LBM is a cereblon binding moiety thereby forming a compound of Formula I-o-4:

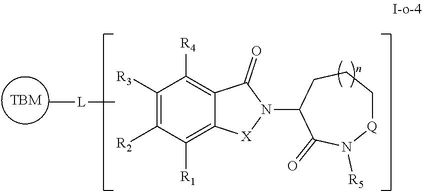

or a pharmaceutically acceptable salt thereof, wherein L and TBM are as defined above and described in embodiments herein, and wherein each of the variables R₁, R₂, R₃, R₄, R₅, Q, X, and n is as described and defined in US 2019/276474, the entirety of each of which is herein incorporated by reference.

In certain embodiments, the present invention provides a compound of Formula I, wherein LBM is a cereblon E3 ubiquitin ligase binding moiety thereby forming a compound of Formula I-o-5, I-o-6, I-o-7 or I-o-8:

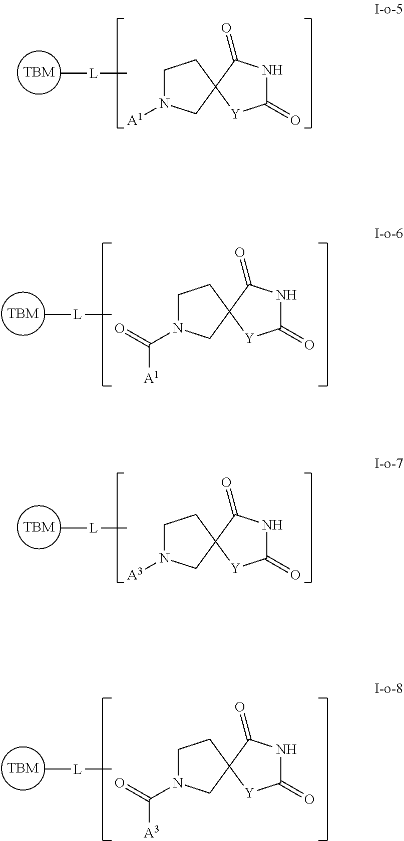

or a pharmaceutically acceptable salt thereof, wherein L and TBM are as defined above and described in embodiments herein, and wherein each of the variables Y, A¹, and A³ is as described and defined in WO 2019/236483, the entirety of each of which is herein incorporated by reference.

In certain embodiments, the present invention provides a compound of Formula I, wherein LBM is human kelch-like ECH-associated protein 1 (KEAP1) of Formula I-o-9:

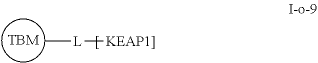

or a pharmaceutically acceptable salt thereof.

In certain embodiments, the present invention provides a compound of Formula I, wherein LBM is KEAP1 binding moiety as recited in Lu et al., Euro. J. Med. Chem., 2018, 146:251-9, thereby forming a compound of Formula I-o-10:

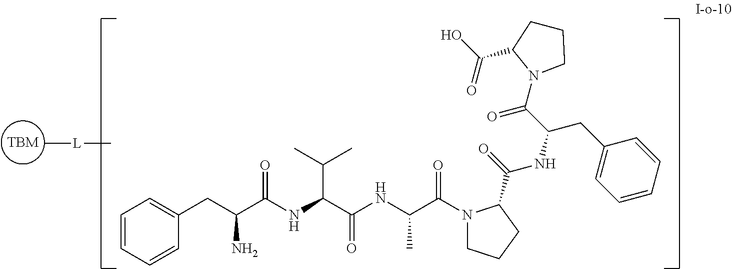

or a pharmaceutically acceptable salt thereof, wherein L and TBM are as defined above and described in embodiments herein.

In certain embodiments, the present invention provides a compound of Formula I, wherein LBM is KEAP1-NRF2 binding moiety thereby forming a compound of Formula I-o-11 or I-o-12:

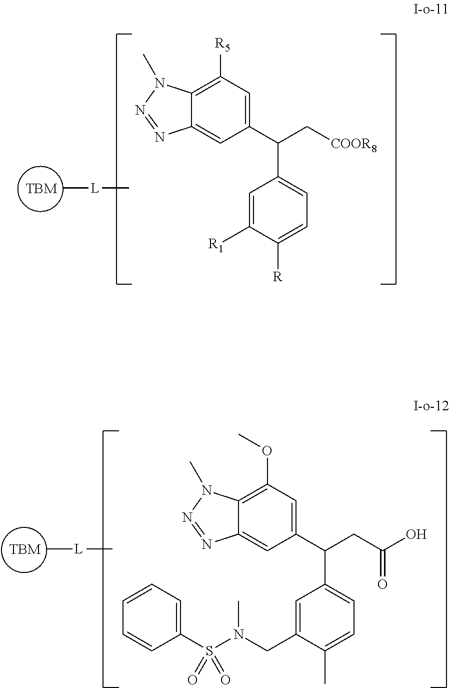

or a pharmaceutically acceptable salt thereof, wherein L and TBM are as defined above and described in embodiments herein, wherein each of the variables R, R₁, R₅, and R₈ is as described and defined in WO 2020/018788, the entirety of each of which is herein incorporated by reference.

In certain embodiments, the present invention provides a compound of Formula I, wherein LBM is KEAP1-NRF2 binding moiety as recited in Tong et al., “Targeted Protein Degradation via a Covalent Reversible Degrader Based on Bardoxolone”, ChemRxiv 2020, thereby forming a compound of Formula I-o-13 or I-o-14:

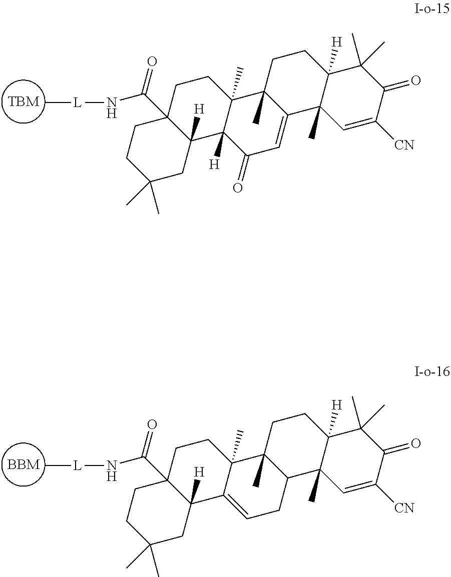

or a pharmaceutically acceptable salt thereof, wherein L and TBM are as defined above and described in embodiments herein.

### Lysine Mimetic

In some embodiments, DIM is LBM as described above and herein. In some embodiments, DIM is a lysine mimetic. In some embodiments, the covalent attachment of ubiquitin to TYK2 protein is achieved through the action of a lysine mimetic. In some embodiments, upon the binding of a compound of Formula I to TYK2, the DIM moiety that mimics a lysine undergoes ubiquitination thereby marking TYK2 for degradation via the Ubiquitin-Proteasome Pathway (UPP).

In some embodiments, DIM is

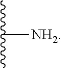 [SMILES: *N]

In some embodiments, DIM is

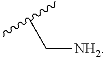 [SMILES: *CN]

In some embodiments, DIM is

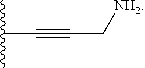 [SMILES: *C#CCN]

In some embodiments, DIM is selected from those depicted in Table 1, below.

In some embodiments, the present invention provides the compound of Formula I as a compound of Formula I-p-1:

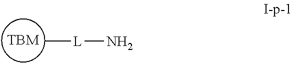

or a pharmaceutically acceptable salt thereof, wherein each of TBM and L is as defined above and described in embodiments herein, both singly and in combination.

In some embodiments, the present invention provides the compound of Formula I as a compound of Formula I-p-2:

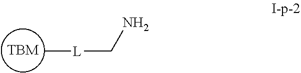

or a pharmaceutically acceptable salt thereof, wherein each of TBM and L is as defined above and described in embodiments herein, both singly and in combination.

In some embodiments, the present invention provides the compound of Formula I as a compound of Formula I-p-3:

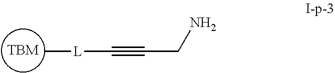

or a pharmaceutically acceptable salt thereof, wherein each of TBM and L is as defined above and described in embodiments herein, both singly and in combination.

In certain embodiments, the present invention provides a compound of Formula I, wherein DIM is a lysine mimetic

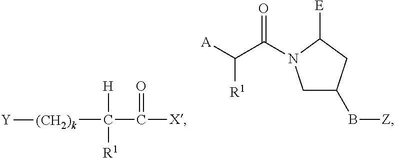

or

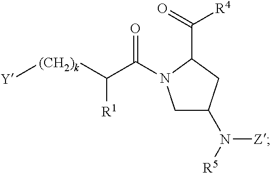

thereby forming a compound of Formulae I-q-1, I-q-2, or I-q-3, respectively:

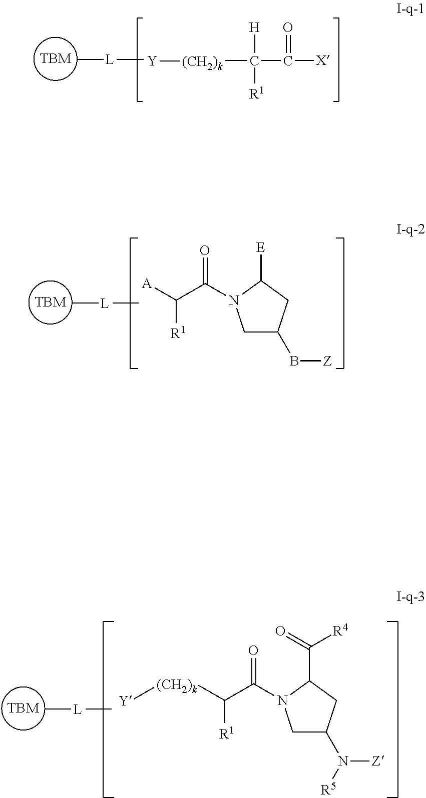

or a pharmaceutically acceptable salt thereof, wherein L and TBM are as defined above and described in embodiments herein, and wherein each of the variables R¹, R⁴, R⁵, A, B, E, Y, Y′, Z, Z′, and k are as defined and described in U.S. Pat. No. 7,622,496, the entirety of each of which is herein incorporated by reference.

### Hydrogen Atom

In some embodiments, DIM is a hydrogen atom. In some embodiments, the covalent attachment of ubiquitin to TYK2 protein is achieved through a provided compound wherein DIM is a hydrogen atom. In some embodiments, upon the binding of a compound of Formula I to TYK2, the DIM moiety being hydrogen effectuates ubiquitination thereby marking TYK2 for degradation via the Ubiquitin-Proteasome Pathway (UPP).

In some embodiments, DIM is selected from those depicted in Table 1, below.

In some embodiments, the present invention provides the compound of Formula I wherein DIM is a hydrogen atom, thereby forming a compound of Formula I-r:

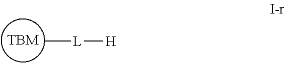

or a pharmaceutically acceptable salt thereof, wherein each of TBM and L is as defined above and described in embodiments herein, both singly and in combination.

### Linker (L)

As defined above and described herein, L is a bivalent moiety that connects to TBM to DIM.

In some embodiments, L is a bivalent moiety that connects TBM to DIM. In some embodiments, L is a bivalent moiety that connects TBM to LBM. In some embodiments, L is a bivalent moiety that connects TBM to a lysine mimetic.

In some embodiments, L is a covalent bond or a bivalent, saturated or partially unsaturated, straight or branched C₁₋₅₀ hydrocarbon chain, wherein 0-10 methylene units of L are independently replaced by —C(D)(H)—, —C(D)₂-, -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

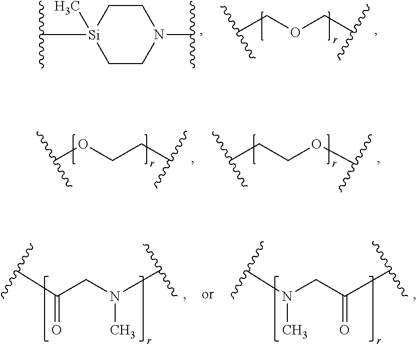

wherein each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur, wherein r is 0, 1, 2, 3, 4, 5, 6, 7, 8, 9, or 10, and wherein R is as defined and described herein.

In some embodiments, L is a covalent bond or a bivalent, saturated or partially unsaturated, straight or branched C₁₋₅₀ hydrocarbon chain, wherein 0-10 methylene units of L are independently replaced by —C(D)(H)—, —C(D)₂-, -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

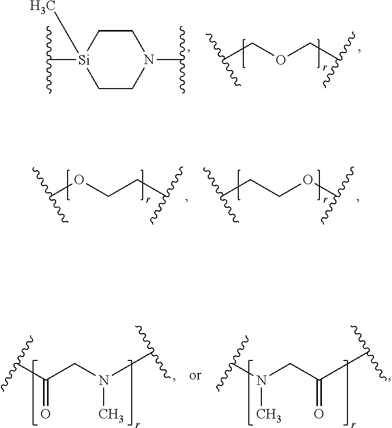

wherein each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur, wherein
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
r is 0, 1, 2, 3, 4, 5, 6, 7, 8, 9, or 10.

In some embodiments, each -Cy- is independently an optionally substituted bivalent phenylenyl. In some embodiments, each -Cy- is independently an optionally substituted 8-10 membered bicyclic arylenyl. In some embodiments, each -Cy- is independently an optionally substituted 4-7 membered saturated or partially unsaturated carbocyclylenyl. In some embodiments, each -Cy- is independently an optionally substituted 4-11 membered saturated or partially unsaturated spiro carbocyclylenyl. In some embodiments, each -Cy- is independently an optionally substituted 8-10 membered bicyclic saturated or partially unsaturated carbocyclylenyl. In some embodiments, each -Cy- is independently an optionally substituted 4-7 membered saturated or partially unsaturated heterocyclylenyl having 1-2 heteroatoms independently selected from nitrogen, oxygen, and sulfur. In some embodiments, each -Cy- is independently an optionally substituted 4-11 membered saturated or partially unsaturated spiro heterocyclylenyl having 1-2 heteroatoms independently selected from nitrogen, oxygen, and sulfur. In some embodiments, each -Cy- is independently an optionally substituted 8-10 membered bicyclic saturated or partially unsaturated heterocyclylenyl having 1-2 heteroatoms independently selected from nitrogen, oxygen, and sulfur. In some embodiments, each -Cy- is independently an optionally substituted 5-6 membered heteroarylenyl having 1-4 heteroatoms independently selected from nitrogen, oxygen, and sulfur. In some embodiments, each -Cy- is independently an optionally substituted 8-10 membered bicyclic heteroarylenyl having 1-5 heteroatoms independently selected from nitrogen, oxygen, and sulfur.

In some embodiments, L is a covalent bond or a bivalent, saturated or partially unsaturated, straight or branched C₁-C₂₀ alkylene chain, wherein 0-6 methylene units of L are independently replaced by -Cy-, —O—, —NR^{C}—, —S—, —OC(O)—, —C(O)O—, —C(O)—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—, and combinations thereof,
wherein -Cy- is independently at each occurrence an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl, having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur, and
each R^{C} is independently at each occurrence hydrogen, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, and combinations thereof.

In some embodiments, -Cy- is

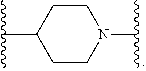 [SMILES: *C1CCN(*)CC1]

In some embodiments, -Cy- is

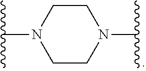 [SMILES: *N1CCN(*)CC1]

In some embodiments, -Cy- is

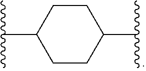 [SMILES: *C1CCC(*)CC1]

In some embodiments, -Cy- is

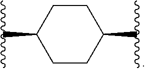 [SMILES: *[C@H]1CC[C@@H](*)CC1]

In some embodiments, -Cy- is

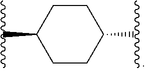 [SMILES: *[C@H]1CC[C@H](*)CC1]

In some embodiments, -Cy- is

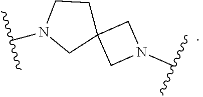 [SMILES: *N1CCC2(C1)CN(*)C2]

In some embodiments, -Cy- is

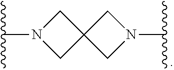 [SMILES: *N1CC2(C1)CN(*)C2]

In some embodiments, -Cy- is

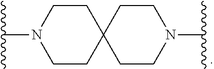 [SMILES: *N1CCC2(CC1)CCN(*)CC2]

In some embodiments, -Cy- is substituted with C₁₋₆ alkyl (e.g., methyl, ethyl, isopropyl). In some embodiments, -Cy- is substituted with oxo. In some embodiments, -Cy- is substituted with halogen. In some embodiments, -Cy- is substituted with fluoro. In some embodiments, -Cy- is substituted with geminal difluoro. In some embodiments, -Cy- is substituted with —OH. In some embodiments, -Cy- is substituted with —NR₂.

In some embodiments, -Cy- is selected from those as depicted in the compounds of Table 1, below.

In some embodiments, r is 0. In some embodiments, r is 1. In some embodiments, r is 2. In some embodiments, r is 3. In some embodiments, r is 4. In some embodiments, r is 5. In some embodiments, r is 6. In some embodiments, r is 7. In some embodiments, r is 8. In some embodiments, r is 9. In some embodiments, r is 10.

In some embodiments, r is selected from those depicted in Table 1 below.

In some embodiments, L is —NR—(C₁₋₁₀ aliphatic)-. In some embodiments, L is —(C₁₋₁₀ aliphatic)-NR—(C₁₋₁₀ aliphatic)-. In some embodiments, L is —(C₁₋₁₀ aliphatic)-NR—(CH₂CH₂O)₁₋₁₀CH₂CH₂—. In some embodiments, L is -Cy-NR—(C₁₋₁₀ aliphatic)-. In some embodiments, L is -Cy-(C₁₋₁₀ aliphatic)-NR—. In some embodiments, L is -Cy-(C₁₋₁₀ aliphatic)-NR—(C₁₋₁₀ aliphatic)-. In some embodiments, L is —(C₁₋₁₀ aliphatic)-Cy-NR—(C₁₋₁₀ aliphatic)-. In some embodiments, L is —(C₁₋₁₀ aliphatic)-Cy-(C₁₋₁₀ aliphatic)-NR—. In some embodiments, L is —(C₁₋₁₀ aliphatic)-Cy-(C₁₋₁₀ aliphatic)-NR—(C₁₋₁₀ aliphatic)-. In some embodiments, L is -Cy-(C₁₋₁₀ aliphatic)-Cy-NR—. In some embodiments, L is -Cy-(C₁₋₁₀ aliphatic)-NR-Cy-. In some embodiments, L is -Cy-(C₁₋₁₀ aliphatic)-Cy-NR—(C₁₋₁₀ aliphatic)-. In some embodiments, L is -Cy-(C₁₋₁₀ aliphatic)-NR-Cy-(C₁₋₁₀ aliphatic)-.

In some embodiments, L is —CONR—(C₁₋₁₀ aliphatic)-. In some embodiments, L is —(C₁₋₁₀ aliphatic)-CONR—(C₁₋₁₀ aliphatic)-. In some embodiments, L is —(C₁₋₁₀ aliphatic)-CONR—(CH₂CH₂O)₁₋₁₀CH₂CH₂—. In some embodiments, L is -Cy-CONR—(C₁₋₁₀ aliphatic)-. In some embodiments, L is -Cy-(C₁₋₁₀ aliphatic)-CONR—. In some embodiments, L is -Cy-(C₁₋₁₀ aliphatic)-CONR—(C₁₋₁₀ aliphatic)-. In some embodiments, L is —(C₁₋₁₀ aliphatic)-Cy-CONR—(C₁₋₁₀ aliphatic)-. In some embodiments, L is —(C₁₋₁₀ aliphatic)-Cy-(C₁₋₁₀ aliphatic)-CONR—. In some embodiments, L is —(C₁₋₁₀ aliphatic)-Cy-(C₁₋₁₀ aliphatic)-CONR—(C₁₋₁₀ aliphatic)-. In some embodiments, L is -Cy-(C₁₋₁₀ aliphatic)-Cy-CONR—. In some embodiments, L is -Cy-(C₁₋₁₀ aliphatic)-CONR-Cy-. In some embodiments, L is -Cy-(C₁₋₁₀ aliphatic)-Cy-CONR—(C₁₋₁₀ aliphatic)-. In some embodiments, L is -Cy-(C₁₋₁₀ aliphatic)-CONR-Cy-(C₁₋₁₀ aliphatic)-.

In some embodiments, L is —NRCO—(C₁₋₁₀ aliphatic)-. In some embodiments, L is —(C₁₋₁₀ aliphatic)-NRCO—(C₁₋₁₀ aliphatic)-. In some embodiments, L is —(C₁₋₁₀ aliphatic)-NRCO—(CH₂CH₂O)₁₋₁₀CH₂CH₂—. In some embodiments, L is -Cy-NRCO—(C₁₋₁₀ aliphatic)-. In some embodiments, L is -Cy-(C₁₋₁₀ aliphatic)-NRCO—. In some embodiments, L is -Cy-(C₁₋₁₀ aliphatic)-NRCO—(C₁₋₁₀ aliphatic)-. In some embodiments, L is —(C₁₋₁₀ aliphatic)-Cy-NRCO—(C₁₋₁₀ aliphatic)-. In some embodiments, L is —(C₁₋₁₀ aliphatic)-Cy-(C₁₋₁₀ aliphatic)-NRCO—. In some embodiments, L is —(C₁₋₁₀ aliphatic)-Cy-(C₁₋₁₀ aliphatic)-NRCO—(C₁₋₁₀ aliphatic)-. In some embodiments, L is -Cy-(C₁₋₁₀ aliphatic)-Cy-NRCO—. In some embodiments, L is -Cy-(C₁₋₁₀ aliphatic)-NRCO-Cy-. In some embodiments, L is -Cy-(C₁₋₁₀ aliphatic)-Cy-NRCO—(C₁₋₁₀ aliphatic)-. In some embodiments, L is -Cy-(C₁₋₁₀ aliphatic)-NRCO-Cy-(C₁₋₁₀ aliphatic)-.

In some embodiments, L is —O—(C₁₋₁₀ aliphatic)-. In some embodiments, L is —(C₁₋₁₀ aliphatic)-O—(C₁₋₁₀ aliphatic)-. In some embodiments, L is —(C₁₋₁₀ aliphatic)-O—(CH₂CH₂O)₁₋₁₀CH₂CH₂—. In some embodiments, L is -Cy-O—(C₁₋₁₀ aliphatic)-. In some embodiments, L is -Cy-(C₁₋₁₀ aliphatic)-O—. In some embodiments, L is -Cy-(C₁₋₁₀ aliphatic)-O—(C₁₋₁₀ aliphatic)-. In some embodiments, L is —(C₁₋₁₀ aliphatic)-Cy-O—(C₁₋₁₀ aliphatic)-. In some embodiments, L is —(C₁₋₁₀ aliphatic)-Cy-(C₁₋₁₀ aliphatic)-O—. In some embodiments, L is —(C₁₋₁₀ aliphatic)-Cy-(C₁₋₁₀ aliphatic)-O—(C₁₋₁₀ aliphatic)-. In some embodiments, L is -Cy-(C₁₋₁₀ aliphatic)-Cy-O—. In some embodiments, L is -Cy-(C₁₋₁₀ aliphatic)-O-Cy-. In some embodiments, L is -Cy-(C₁₋₁₀ aliphatic)-Cy-O—(C₁₋₁₀ aliphatic)-. In some embodiments, L is -Cy-(C₁₋₁₀ aliphatic)-O-Cy-(C₁₋₁₀ aliphatic)-.

In some embodiments, L is -Cy-(C₁₋₁₀ aliphatic)-. In some embodiments, L is —(C₁₋₁₀ aliphatic)-Cy-(C₁₋₁₀ aliphatic)-. In some embodiments, L is —(C₁₋₁₀ aliphatic)-Cy-(CH₂CH₂O)₁₋₁₀CH₂CH₂—. In some embodiments, L is -Cy-(C₁₋₁₀ aliphatic)-Cy-. In some embodiments, L is -Cy-(C₁₋₁₀ aliphatic)-Cy-(C₁₋₁₀ aliphatic)-. In some embodiments, L is -Cy-(C₁₋₁₀ aliphatic)-Cy-(C₁₋₁₀ aliphatic)-Cy-. In some embodiments, L is —(C₁₋₁₀ aliphatic)-Cy-(C₁₋₁₀ aliphatic)-Cy-(C₁₋₁₀ aliphatic)-.

In some embodiments, L is —NR—(CH₂)₁₋₁₀—. In some embodiments, L is —(CH₂)₁₋₁₀—NR—(CH₂)₁₋₁₀—. In some embodiments, L is —(CH₂)₁₋₁₀—NR—(CH₂CH₂O)₁₋₁₀CH₂CH₂—. In some embodiments, L is -Cy-NR—(CH₂)₁₋₁₀—. In some embodiments, L is -Cy-(CH₂)₁₋₁₀—NR—. In some embodiments, L is -Cy-(CH₂)₁₋₁₀—NR—(CH₂)₁₋₁₀—. In some embodiments, L is —(CH₂)₁₋₁₀-Cy-NR—(CH₂)₁₋₁₀—. In some embodiments, L is —(CH₂)₁₋₁₀-Cy-(CH₂)₁₋₁₀—NR—. In some embodiments, L is —(CH₂)₁₋₁₀-Cy-(CH₂)₁₋₁₀—NR—(CH₂)₁₋₁₀—. In some embodiments, L is -Cy-(CH₂)₁₋₁₀-Cy-NR—. In some embodiments, L is -Cy-(CH₂)₁₋₁₀—NR-Cy-. In some embodiments, L is -Cy-(CH₂)₁₋₁₀-Cy-NR—(CH₂)₁₋₁₀—. In some embodiments, L is -Cy-(CH₂)₁₋₁₀—NR-Cy-(CH₂)₁₋₁₀—.

In some embodiments, L is —CONR—(CH₂)₁₋₁₀—. In some embodiments, L is —(CH₂)₁₋₁₀—CONR—(CH₂)₁₋₁₀—. In some embodiments, L is —(CH₂)₁₋₁₀—CONR—(CH₂CH₂O)₁₋₁₀CH₂CH₂—. In some embodiments, L is -Cy-CONR—(CH₂)₁₋₁₀—. In some embodiments, L is -Cy-(CH₂)₁₋₁₀—CONR—. In some embodiments, L is -Cy-(CH₂)₁₋₁₀—CONR—(CH₂)₁₋₁₀—. In some embodiments, L is —(CH₂)₁₋₁₀-Cy-CONR—(CH₂)₁₋₁₀—. In some embodiments, L is —(CH₂)₁₋₁₀-Cy-(CH₂)₁₋₁₀—CONR—. In some embodiments, L is —(CH₂)₁₋₁₀-Cy-(CH₂)₁₋₁₀—CONR—(CH₂)₁₋₁₀—. In some embodiments, L is -Cy-(CH₂)₁₋₁₀-Cy-CONR—. In some embodiments, L is -Cy-(CH₂)₁₋₁₀—CONR-Cy-. In some embodiments, L is -Cy-(CH₂)₁₋₁₀-Cy-CONR—(CH₂)₁₋₁₀—. In some embodiments, L is -Cy-(CH₂)₁₋₁₀—CONR-Cy-(CH₂)₁₋₁₀—.

In some embodiments, L is —NRCO—(CH₂)₁₋₁₀—. In some embodiments, L is —(CH₂)₁₋₁₀—NRCO—(CH₂)₁₋₁₀—. In some embodiments, L is —(CH₂)₁₋₁₀—NRCO—(CH₂CH₂O)₁₋₁₀CH₂CH₂—. In some embodiments, L is -Cy-NRCO—(CH₂)₁₋₁₀—. In some embodiments, L is -Cy-(CH₂)₁₋₁₀—NRCO—. In some embodiments, L is -Cy-(CH₂)₁₋₁₀—NRCO—(CH₂)₁₋₁₀—. In some embodiments, L is —(CH₂)₁₋₁₀-Cy-NRCO—(CH₂)₁₋₁₀—. In some embodiments, L is —(CH₂)₁₋₁₀-Cy-(CH₂)₁₋₁₀—NRCO—. In some embodiments, L is —(CH₂)₁₋₁₀-Cy-(CH₂)₁₋₁₀—NRCO—(CH₂)₁₋₁₀—. In some embodiments, L is -Cy-(CH₂)₁₋₁₀-Cy-NRCO—. In some embodiments, L is -Cy-(CH₂)₁₋₁₀—NRCO-Cy-. In some embodiments, L is -Cy-(CH₂)₁₋₁₀-Cy-NRCO—(CH₂)₁₋₁₀—. In some embodiments, L is -Cy-(CH₂)₁₋₁₀—NRCO-Cy-(CH₂)₁₋₁₀—.

In some embodiments, L is —O—(CH₂)₁₋₁₀—. In some embodiments, L is —(CH₂)₁₋₁₀—O—(CH₂)₁₋₁₀—. In some embodiments, L is —(CH₂)₁₋₁₀—O—(CH₂CH₂O)₁₋₁₀CH₂CH₂—. In some embodiments, L is -Cy-O—(CH₂)₁₋₁₀—. In some embodiments, L is -Cy-(CH₂)₁₋₁₀—O—. In some embodiments, L is -Cy-(CH₂)₁₋₁₀—O—(CH₂)₁₋₁₀—. In some embodiments, L is —(CH₂)₁₋₁₀-Cy-O—(CH₂)₁₋₁₀—. In some embodiments, L is —(CH₂)₁₋₁₀-Cy-(CH₂)₁₋₁₀—O—. In some embodiments, L is —(CH₂)₁₋₁₀-Cy-(CH₂)₁₋₁₀—O—(CH₂)₁₋₁₀—. In some embodiments, L is -Cy-(CH₂)₁₋₁₀-Cy-O—. In some embodiments, L is -Cy-(CH₂)₁₋₁₀—O-Cy-. In some embodiments, L is -Cy-(CH₂)₁₋₁₀-Cy-O—(CH₂)₁₋₁₀—. In some embodiments, L is -Cy-(CH₂)₁₋₁₀—O-Cy-(CH₂)₁₋₁₀—.

In some embodiments, L is -Cy-(CH₂)₁₋₁₀—. In some embodiments, L is —(CH₂)₁₋₁₀-Cy-(CH₂)₁₋₁₀—. In some embodiments, L is —(CH₂)₁₋₁₀-Cy-(CH₂CH₂O)₁₋₁₀CH₂CH₂—. In some embodiments, L is -Cy-(CH₂)₁₋₁₀-Cy-. In some embodiments, L is -Cy-(CH₂)₁₋₁₀-Cy-(CH₂)₁₋₁₀—. In some embodiments, L is -Cy-(CH₂)₁₋₁₀-Cy-(CH₂)₁₋₁₀-Cy-. In some embodiments, L is —(CH₂)₁₋₁₀-Cy-(CH₂)₁₋₁₀-Cy-(CH₂)₁₋₁₀—.

In some embodiments, L is

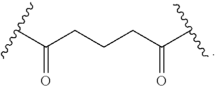 [SMILES: *C(=O)CCCC(*)=O]

In some embodiments, L is

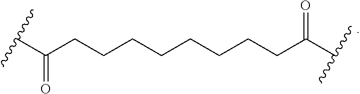 [SMILES: *C(=O)CCCCCCCCC(*)=O]

In some embodiments, L is

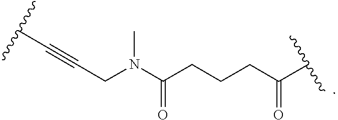 [SMILES: *C#CCN(C)C(=O)CCCC(*)=O]

In some embodiments, L is

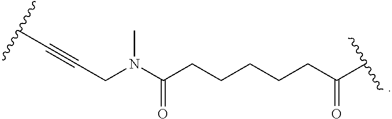 [SMILES: *C#CCN(C)C(=O)CCCCCC(*)=O]

In some embodiments, L is

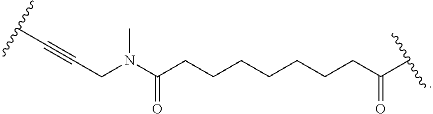 [SMILES: *C#CCN(C)C(=O)CCCCCCCC(*)=O]

In some embodiments, L is

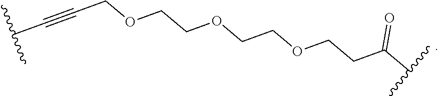 [SMILES: *C#CCOCCOCCOCCC(*)=O]

In some embodiments, L is

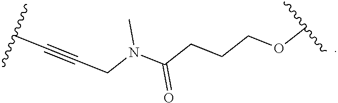 [SMILES: *C#CCN(C)C(=O)CCCO*]

In some embodiments, L is

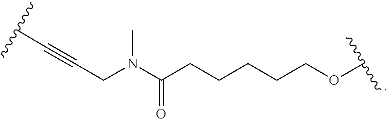 [SMILES: *C#CCN(C)C(=O)CCCCCO*]

In some embodiments, L is

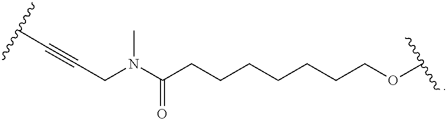 [SMILES: *C#CCN(C)C(=O)CCCCCCCO*]

In some embodiments, L is

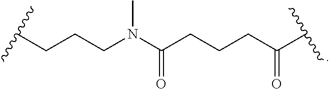 [SMILES: *CCCN(C)C(=O)CCCC(*)=O]

In some embodiments, L is

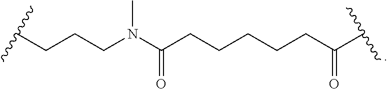 [SMILES: *CCCN(C)C(=O)CCCCCC(*)=O]

In some embodiments, L is

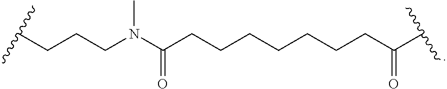 [SMILES: *CCCN(C)C(=O)CCCCCCCC(*)=O]

In some embodiments, L is

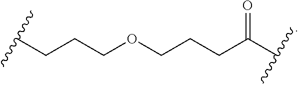 [SMILES: *CCCOCCCC(*)=O]

In some embodiments, L is

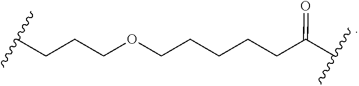 [SMILES: *CCCOCCCCCC(*)=O]

In some embodiments, L is

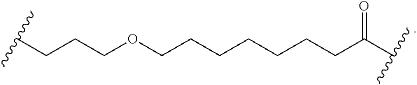 [SMILES: *CCCOCCCCCCCC(*)=O]

In some embodiments, L is

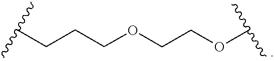 [SMILES: *CCCOCCO*]

In some embodiments, L is

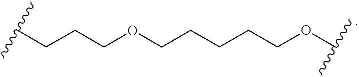 [SMILES: *CCCOCCCCCO*]

In some embodiments, L is

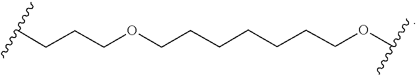 [SMILES: *CCCOCCCCCCCO*]

In some embodiments, L is

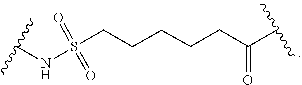 [SMILES: *NS(=O)(=O)CCCCCC(*)=O]

In some embodiments, L is

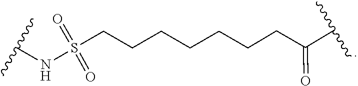 [SMILES: *NS(=O)(=O)CCCCCCCC(*)=O]

In some embodiments, L is

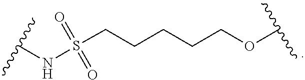 [SMILES: *NS(=O)(=O)CCCCCO*]

In some embodiments, L is

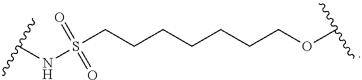 [SMILES: *NS(=O)(=O)CCCCCCCO*]

In some embodiments, L is

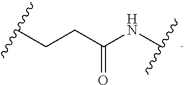 [SMILES: *CCC(=O)N*]

In some embodiments, L is

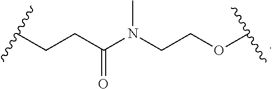 [SMILES: *CCC(=O)N(C)CCO*]

In some embodiments, L is

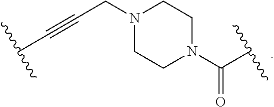 [SMILES: *C#CCN1CCN(C(*)=O)CC1]

In some embodiments, L is

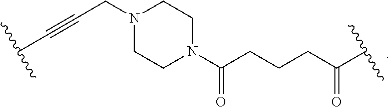 [SMILES: *C#CCN1CCN(C(=O)CCCC(*)=O)CC1]

In some embodiments, L is

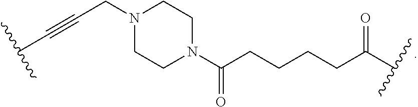 [SMILES: *C#CCN1CCN(C(=O)CCCCC(*)=O)CC1]

In some embodiments, L is

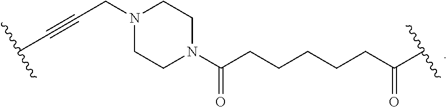 [SMILES: *C#CCN1CCN(C(=O)CCCCCC(*)=O)CC1]

In some embodiments, L is

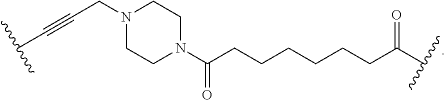 [SMILES: *C#CCN1CCN(C(=O)CCCCCCC(*)=O)CC1]

In some embodiments, L is

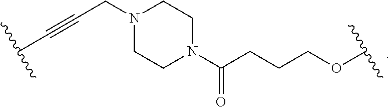 [SMILES: *C#CCN1CCN(C(=O)CCCO*)CC1]

In some embodiments, L is

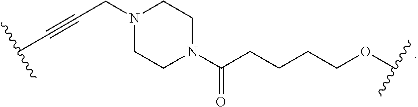 [SMILES: *C#CCN1CCN(C(=O)CCCCO*)CC1]

In some embodiments, L is

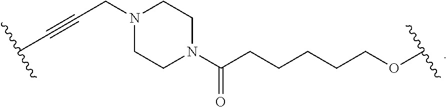 [SMILES: *C#CCN1CCN(C(=O)CCCCCO*)CC1]

In some embodiments, L is

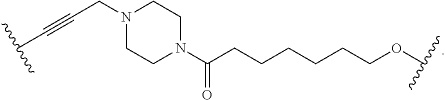 [SMILES: *C#CCN1CCN(C(=O)CCCCCCO*)CC1]

In some embodiments, L is

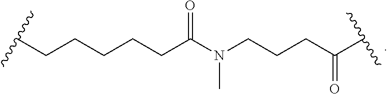 [SMILES: *CCCCCC(=O)N(C)CCCC(*)=O]

In some embodiments, L is

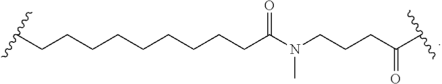 [SMILES: *CCCCCCCCCC(=O)N(C)CCCC(*)=O]

In some embodiments, L is

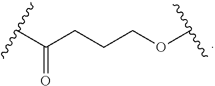 [SMILES: *OCCCC(*)=O]

In some embodiments, L is

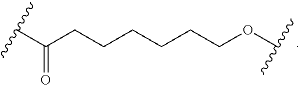 [SMILES: *OCCCCCCC(*)=O]

In some embodiments, L is

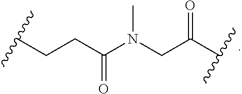 [SMILES: *CCC(=O)N(C)CC(*)=O]

In some embodiments, L is

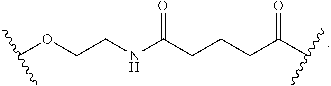 [SMILES: *OCCNC(=O)CCCC(*)=O]

In some embodiments, L is

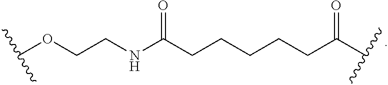 [SMILES: *OCCNC(=O)CCCCCC(*)=O]

In some embodiments, L is

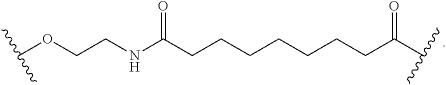 [SMILES: *OCCNC(=O)CCCCCCCC(*)=O]

In some embodiments, L is

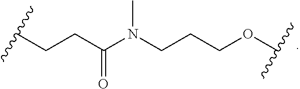 [SMILES: *CCC(=O)N(C)CCCO*]

In some embodiments, L is

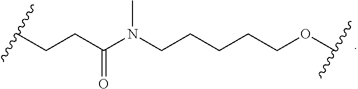 [SMILES: *CCC(=O)N(C)CCCCCO*]

In some embodiments, L is

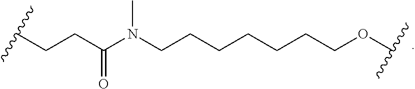 [SMILES: *CCC(=O)N(C)CCCCCCCO*]

In some embodiments, L is

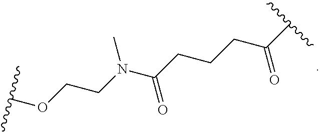 [SMILES: *OCCN(C)C(=O)CCCC(*)=O]

In some embodiments, L is

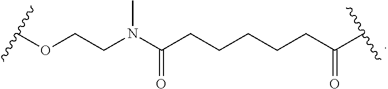 [SMILES: *OCCN(C)C(=O)CCCCCC(*)=O]

In some embodiments, L is

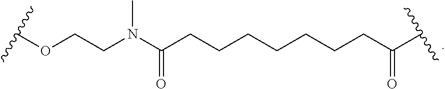 [SMILES: *OCCN(C)C(=O)CCCCCCCC(*)=O]

In some embodiments, L is

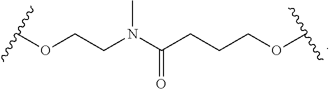 [SMILES: *OCCCC(=O)N(C)CCO*]

In some embodiments, L is

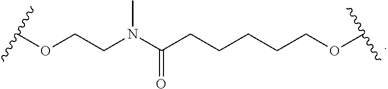 [SMILES: *OCCCCCC(=O)N(C)CCO*]

In some embodiments, L is

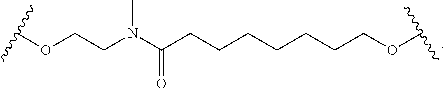 [SMILES: *OCCCCCCCC(=O)N(C)CCO*]

In some embodiments, L is

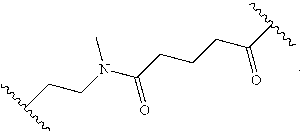 [SMILES: *CCN(C)C(=O)CCCC(*)=O]

In some embodiments, L is

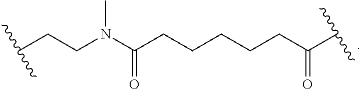 [SMILES: *CCN(C)C(=O)CCCCCC(*)=O]

In some embodiments, L is

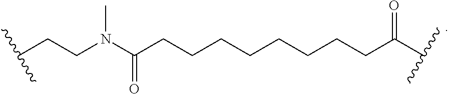 [SMILES: *CCN(C)C(=O)CCCCCCCCC(*)=O]

In some embodiments, L is

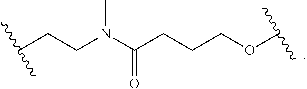 [SMILES: *CCN(C)C(=O)CCCO*]

In some embodiments, L is

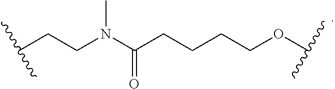 [SMILES: *CCN(C)C(=O)CCCCO*]

In some embodiments, L is

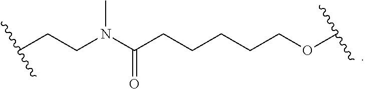 [SMILES: *CCN(C)C(=O)CCCCCO*]

In some embodiments, L is

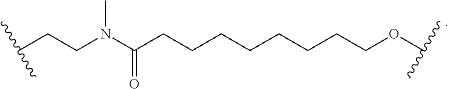 [SMILES: *CCN(C)C(=O)CCCCCCCCO*]

In some embodiments, L is

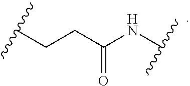 [SMILES: *CCC(=O)N*]

In some embodiments, L is

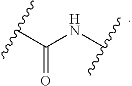 [SMILES: *NC(*)=O]

In some embodiments, L is

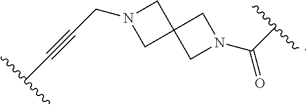 [SMILES: *C#CCN1CC2(C1)CN(C(*)=O)C2]

In some embodiments, L is

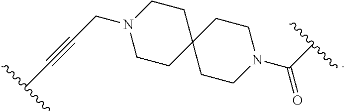 [SMILES: *C#CCN1CCC2(CC1)CCN(C(*)=O)CC2]

In some embodiments, L is

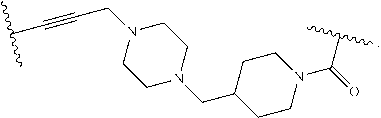 [SMILES: *C#CCN1CCN(CC2CCN(C(*)=O)CC2)CC1]

In some embodiments, L is

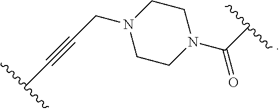 [SMILES: *C#CCN1CCN(C(*)=O)CC1]

In some embodiments, L is

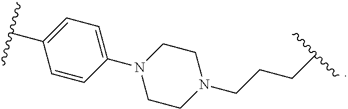 [SMILES: *CCCN1CCN(c2ccc(*)cc2)CC1]

In some embodiments, L is

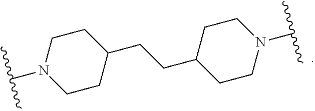 [SMILES: *N1CCC(CCC2CCN(*)CC2)CC1]

In some embodiments, L is

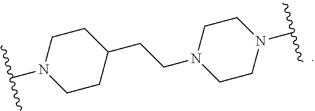 [SMILES: *N1CCC(CCN2CCN(*)CC2)CC1]

In some embodiments, L is

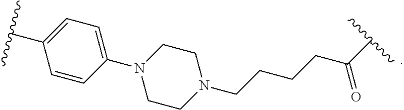 [SMILES: *C(=O)CCCCN1CCN(c2ccc(*)cc2)CC1]

In some embodiments, L is

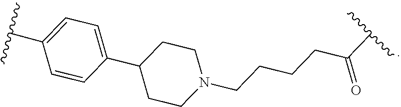 [SMILES: *C(=O)CCCCN1CCC(c2ccc(*)cc2)CC1]

In some embodiments, L is

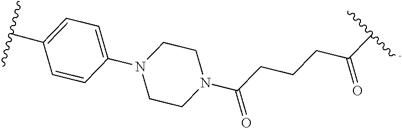 [SMILES: *C(=O)CCCC(=O)N1CCN(c2ccc(*)cc2)CC1]

In some embodiments, L is

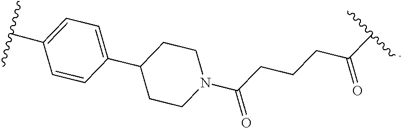 [SMILES: *C(=O)CCCC(=O)N1CCC(c2ccc(*)cc2)CC1]

In some embodiments, L is

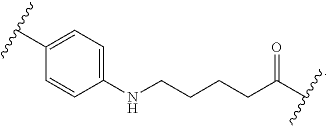 [SMILES: *C(=O)CCCCNc1ccc(*)cc1]

In some embodiments, L is

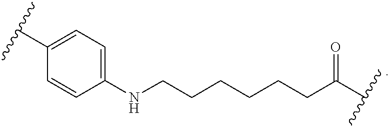 [SMILES: *C(=O)CCCCCCNc1ccc(*)cc1]

In some embodiments, L is

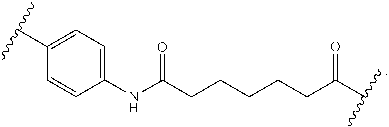 [SMILES: *C(=O)CCCCCC(=O)Nc1ccc(*)cc1]

In some embodiments, L is

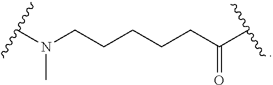 [SMILES: *C(=O)CCCCCN(*)C]

In some embodiments L is

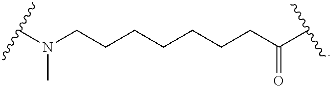 [SMILES: *C(=O)CCCCCCCN(*)C]

In some embodiments, L is

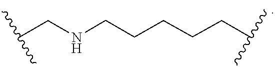 [SMILES: *CCCCCNC*]

In some embodiments, L is

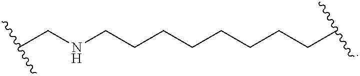 [SMILES: *CCCCCCCCNC*]

In some embodiments, L is

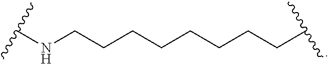 [SMILES: *CCCCCCCCN*]

In some embodiments, L is

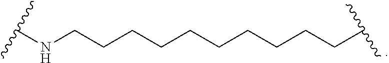 [SMILES: *CCCCCCCCCCN*]

In some embodiments, L is

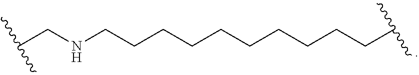 [SMILES: *CCCCCCCCCCNC*]

In some embodiments, L is

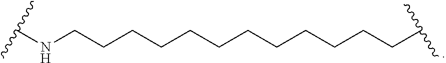 [SMILES: *CCCCCCCCCCCCN*]

In some embodiments, L is

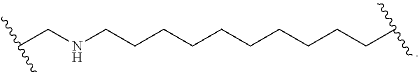 [SMILES: *CCCCCCCCCCNC*]

In some embodiments, L is

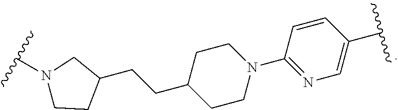 [SMILES: *c1ccc(N2CCC(CCC3CCN(*)C3)CC2)nc1]

In some embodiments, L is

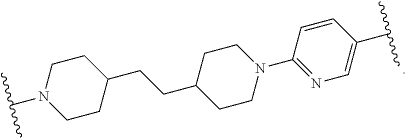 [SMILES: *c1ccc(N2CCC(CCC3CCN(*)CC3)CC2)nc1]

In some embodiments, L is

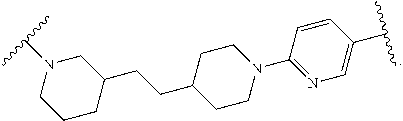 [SMILES: *c1ccc(N2CCC(CCC3CCCN(*)C3)CC2)nc1]

In some embodiments, L is

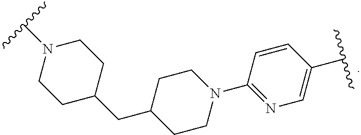 [SMILES: *c1ccc(N2CCC(CC3CCN(*)CC3)CC2)nc1]

In some embodiments, L is

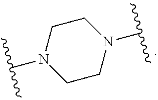 [SMILES: *N1CCN(*)CC1]

In some embodiments, L is

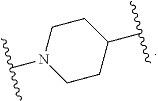 [SMILES: *C1CCN(*)CC1]

In some embodiments, L is

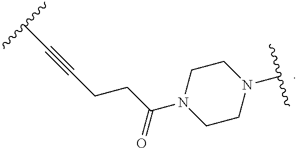 [SMILES: *C#CCCC(=O)N1CCN(*)CC1]

In some embodiments, L is

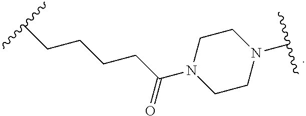 [SMILES: *CCCCC(=O)N1CCN(*)CC1]

In some embodiments, L is

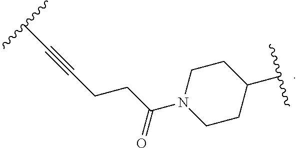 [SMILES: *C#CCCC(=O)N1CCC(*)CC1]

In some embodiments, L is

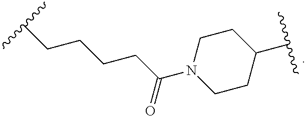 [SMILES: *CCCCC(=O)N1CCC(*)CC1]

In some embodiments, L is

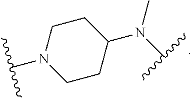 [SMILES: *N1CCC(N(*)C)CC1]

In some embodiments, L is

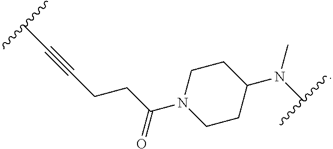 [SMILES: *C#CCCC(=O)N1CCC(N(*)C)CC1]

In some embodiments, L is

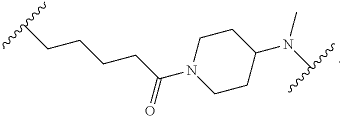 [SMILES: *CCCCC(=O)N1CCC(N(*)C)CC1]

In some embodiments, L is

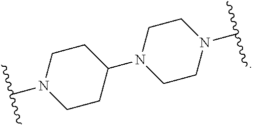 [SMILES: *N1CCC(N2CCN(*)CC2)CC1]

In some embodiments, L is

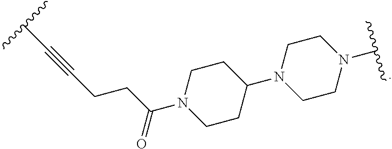 [SMILES: *C#CCCC(=O)N1CCC(N2CCN(*)CC2)CC1]

In some embodiments, L is

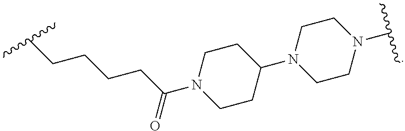 [SMILES: *CCCCC(=O)N1CCC(N2CCN(*)CC2)CC1]

In some embodiments, L is

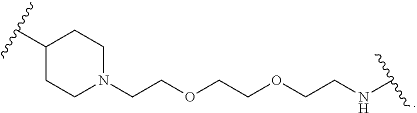 [SMILES: *NCCOCCOCCN1CCC(*)CC1]

In some embodiments, L is

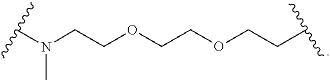 [SMILES: *CCOCCOCCN(*)C]

In some embodiments, L is

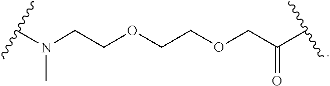 [SMILES: *C(=O)COCCOCCN(*)C]

In some embodiments, L is

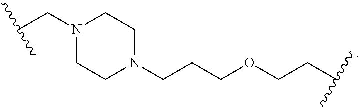 [SMILES: *CCOCCCN1CCN(C*)CC1]

In some embodiments, L is

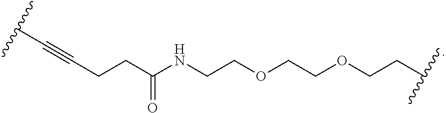 [SMILES: *C#CCCC(=O)NCCOCCOCC*]

In some embodiments, L is

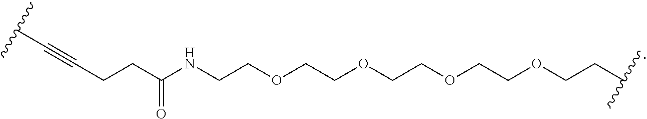 [SMILES: *C#CCCC(=O)NCCOCCOCCOCCOCC*]

In some embodiments, L is

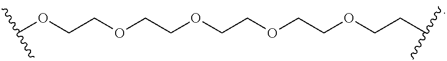 [SMILES: *CCOCCOCCOCCOCCO*]

In some embodiments, L is

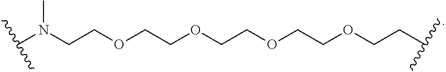 [SMILES: *CCOCCOCCOCCOCCN(*)C]

In some embodiments, L is

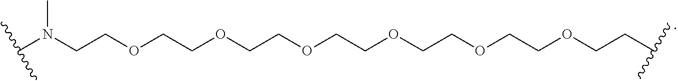 [SMILES: *CCOCCOCCOCCOCCOCCOCCN(*)C]

In some embodiments, L is

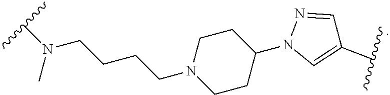 [SMILES: *c1cnn(C2CCN(CCCCN(*)C)CC2)c1]

In some embodiments, L is

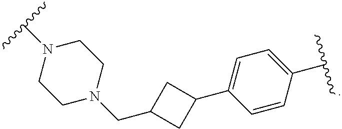 [SMILES: *c1ccc(C2CC(CN3CCN(*)CC3)C2)cc1]

In some embodiments, L is

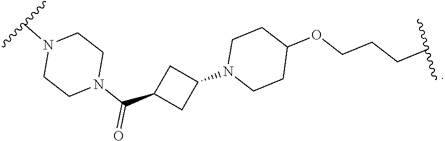 [SMILES: *CCCOC1CCN([C@H]2C[C@H](C(=O)N3CCN(*)CC3)C2)CC1]

In some embodiments, L is

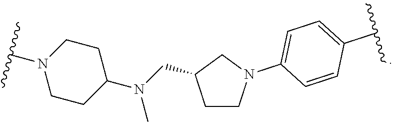 [SMILES: *c1ccc(N2CC[C@H](CN(C)C3CCN(*)CC3)C2)cc1]

In some embodiments, L is

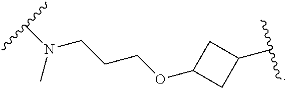 [SMILES: *C1CC(OCCCN(*)C)C1]

In some embodiments, L is

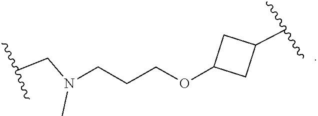 [SMILES: *CN(C)CCCOC1CC(*)C1]

In some embodiments, L is

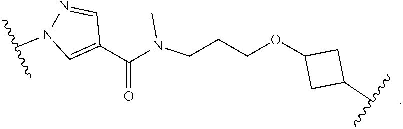 [SMILES: *C1CC(OCCCN(C)C(=O)c2cnn(*)c2)C1]

In some embodiments, L is

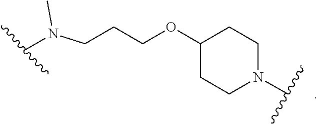 [SMILES: *N(C)CCCOC1CCN(*)CC1]

In some embodiments, L is

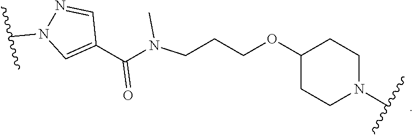 [SMILES: *N1CCC(OCCCN(C)C(=O)c2cnn(*)c2)CC1]

In some embodiments, L is

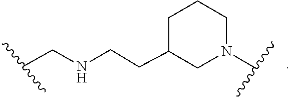 [SMILES: *CNCCC1CCCN(*)C1]

In some embodiments, L is

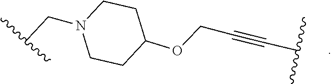 [SMILES: *C#CCOC1CCN(C*)CC1]

In some embodiments, L is

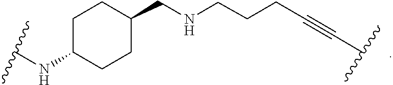 [SMILES: *C#CCCCNC[C@H]1CC[C@H](N*)CC1]

In some embodiments, L is

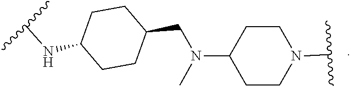 [SMILES: *N[C@H]1CC[C@H](CN(C)C2CCN(*)CC2)CC1]

In some embodiments, L is

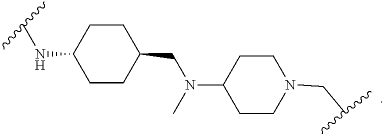 [SMILES: *CN1CCC(N(C)C[C@H]2CC[C@H](N*)CC2)CC1]

In some embodiments, L is

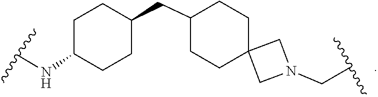 [SMILES: *CN1CC2(CCC(C[C@H]3CC[C@H](N*)CC3)CC2)C1]

In some embodiments, L is

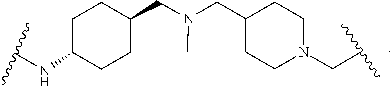 [SMILES: *CN1CCC(CN(C)C[C@H]2CC[C@H](N*)CC2)CC1]

In some embodiments, L is

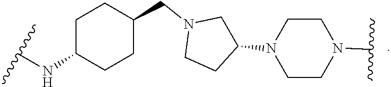 [SMILES: *N[C@H]1CC[C@H](CN2CC[C@@H](N3CCN(*)CC3)C2)CC1]

In some embodiments, L is

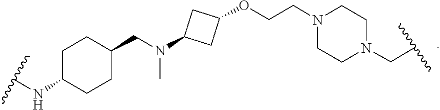 [SMILES: *CN1CCN(CCO[C@H]2C[C@H](N(C)C[C@H]3CC[C@H](N*)CC3)C2)CC1]

In some embodiments, L is

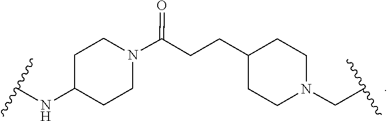 [SMILES: *CN1CCC(CCC(=O)N2CCC(N*)CC2)CC1]

In some embodiments, L is

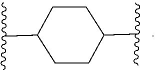 [SMILES: *C1CCC(*)CC1]

In some embodiments, L is

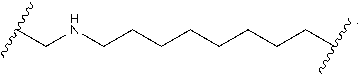 [SMILES: *CCCCCCCCNC*]

In some embodiments, L is

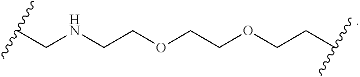 [SMILES: *CCOCCOCCNC*]

In some embodiments L is

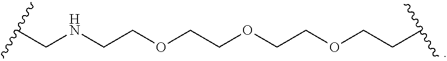 [SMILES: *CCOCCOCCOCCNC*]

In some embodiments, L is

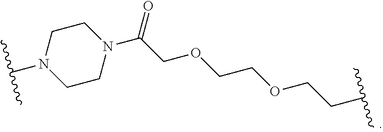 [SMILES: *CCOCCOCC(=O)N1CCN(*)CC1]

In some embodiments, L is

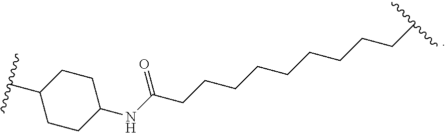 [SMILES: *CCCCCCCCCC(=O)NC1CCC(*)CC1]

In some embodiments, L is

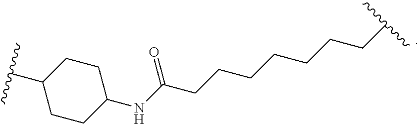 [SMILES: *CCCCCCCC(=O)NC1CCC(*)CC1]

In some embodiments, L is

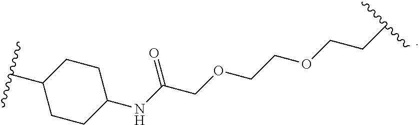 [SMILES: *CCOCCOCC(=O)NC1CCC(*)CC1]

In some embodiments, L is

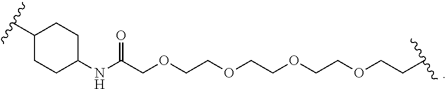 [SMILES: *CCOCCOCCOCCOCC(=O)NC1CCC(*)CC1]

In some embodiments, L is

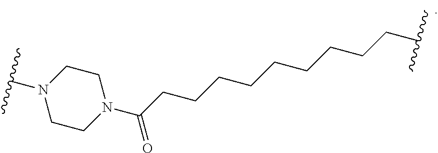 [SMILES: *CCCCCCCCCC(=O)N1CCN(*)CC1]

In some embodiments, L is

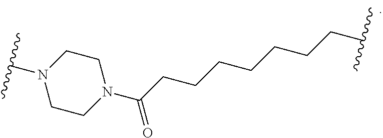 [SMILES: *CCCCCCCC(=O)N1CCN(*)CC1]

In some embodiments, L is

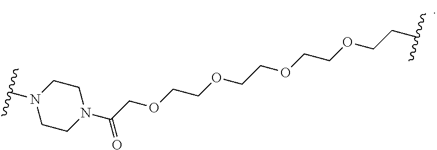 [SMILES: *CCOCCOCCOCCOCC(=O)N1CCN(*)CC1]

In some embodiments, L is

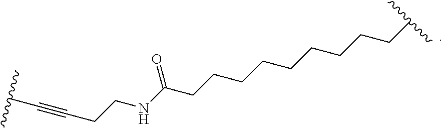 [SMILES: *C#CCCNC(=O)CCCCCCCCC*]

In some embodiments, L is

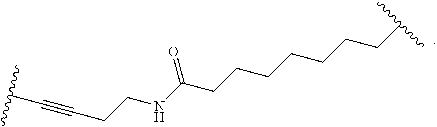 [SMILES: *C#CCCNC(=O)CCCCCCC*]

In some embodiments, L is

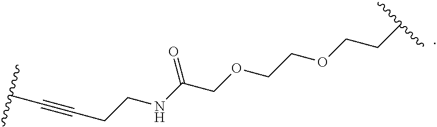 [SMILES: *C#CCCNC(=O)COCCOCC*]

In some embodiments, L is

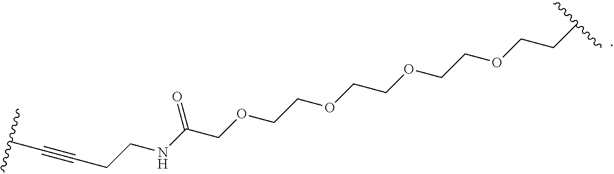 [SMILES: *C#CCCNC(=O)COCCOCCOCCOCC*]

In some embodiments, L is

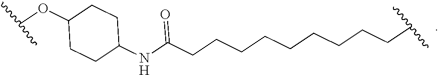 [SMILES: *CCCCCCCCCC(=O)NC1CCC(O*)CC1]

In some embodiments, L is

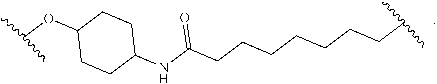 [SMILES: *CCCCCCCC(=O)NC1CCC(O*)CC1]

In some embodiments, L is

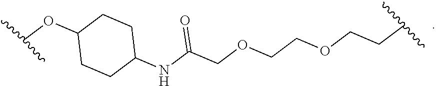 [SMILES: *CCOCCOCC(=O)NC1CCC(O*)CC1]

In some embodiments, L is

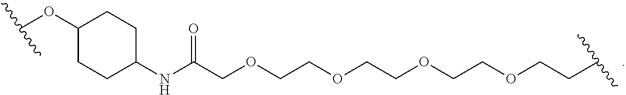 [SMILES: *CCOCCOCCOCCOCC(=O)NC1CCC(O*)CC1]

In some embodiments, L is

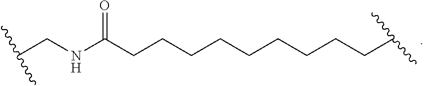 [SMILES: *CCCCCCCCCC(=O)NC*]

In some embodiments, L is

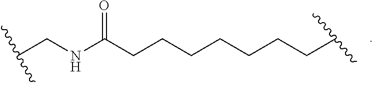 [SMILES: *CCCCCCCC(=O)NC*]

In some embodiments, L is

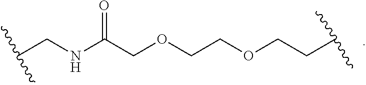 [SMILES: *CCOCCOCC(=O)NC*]

In some embodiments, L is

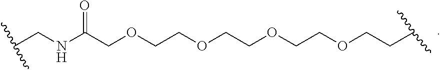 [SMILES: *CCOCCOCCOCCOCC(=O)NC*]

In some embodiments, L is

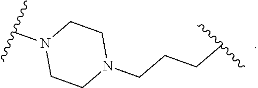 [SMILES: *CCCN1CCN(*)CC1]

In some embodiments, L is

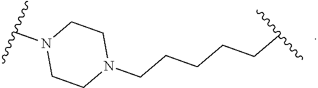 [SMILES: *CCCCCN1CCN(*)CC1]

In some embodiments, L is

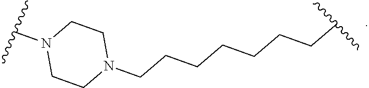 [SMILES: *CCCCCCCN1CCN(*)CC1]

In some embodiments, L is

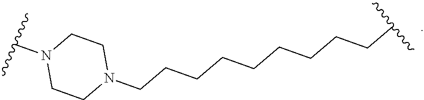 [SMILES: *CCCCCCCCCN1CCN(*)CC1]

In some embodiments, L is

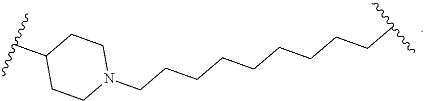 [SMILES: *CCCCCCCCCN1CCC(*)CC1]

In some embodiments, L is

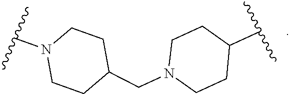 [SMILES: *C1CCN(CC2CCN(*)CC2)CC1]

In some embodiments, L is

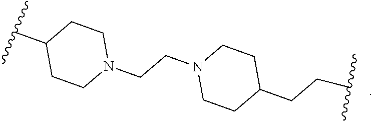 [SMILES: *CCC1CCN(CCN2CCC(*)CC2)CC1]

In some embodiments, L is

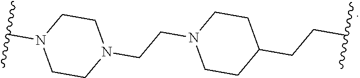 [SMILES: *CCC1CCN(CCN2CCN(*)CC2)CC1]

In some embodiments, L is

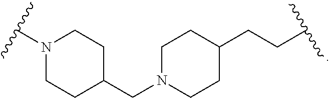 [SMILES: *CCC1CCN(CC2CCN(*)CC2)CC1]

In some embodiments, L is

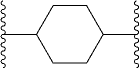 [SMILES: *C1CCC(*)CC1]

In some embodiments, L is selected from those depicted in Table 1, below.

Without limitation, the point of attachment of L to TBM and DIM can be, for example when
L is

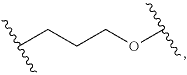 [SMILES: *CCCO*]

either

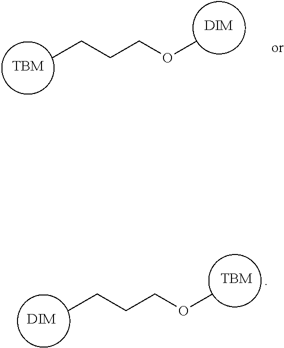

In some embodiments, a provided compound or pharmaceutically acceptable salt thereof, is selected from those wherein TBM is

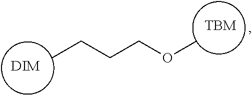

DIM is LBM is selected from any of those in Table A below, and L is selected from any of those in Table B below.

In some embodiments, L is a covalent bond or a bivalent, saturated or partially unsaturated, straight or branched C₁-C₅₀ aliphatic chain, wherein 0-6 methylene units of L are independently replaced by -Cy-, —O—, —NR*—, —S—, —OC(O)—, —C(O)O—, —C(O)—, —S(O)—, —S(O)₂—, —N(R*)S(O)₂—, —S(O)₂N(R*)—, —N(R*)C(O)—, —C(O)N(R*)—, —OC(O)N(R*)—, and —N(R*)C(O)O—, and combinations thereof,
wherein -Cy- is independently at each occurrence an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl, having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur, and
each R* is independently at each occurrence hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, and combinations thereof.

In some embodiments, L is a covalent bond.

In some embodiments, L comprises a saturated straight C₁-C₅₀ aliphatic chain. In some embodiments, L comprises a saturated straight C₁-C₂₀ aliphatic chain. In some embodiments, wherein L comprises a saturated straight C₁-C₁₂ aliphatic chain.

In some embodiments, L comprises a saturated straight C₁-C₈ alkylene chain.

In some embodiments, L comprises a saturated straight C₂-C₆ alkylene chain.

In some embodiments, L comprises a saturated straight C₄ alkylene chain.

In some embodiments, 0-5 methylene units of L are independently replaced by -Cy-, —O—, —NR^{C}—, —N(R^{C})C(O)—, —C(O)N(R^{C})—, —OC(O)N(R^{C})—, and —N(R^{C})C(O)O—, and combinations thereof,

In some embodiments, L comprises a polyethylene glycol (PEG)

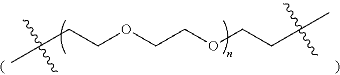

chain, wherein n is an integer from 1 to 10.

In some embodiments, L comprises at least one -Cy-.

In some embodiments, L comprises a structure selected from the group consisting of:

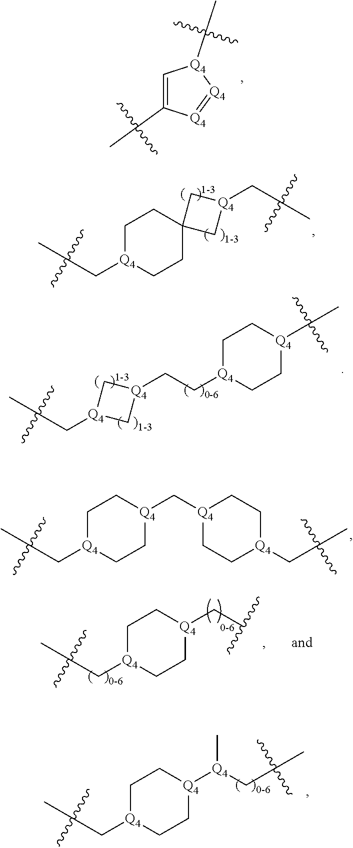

where each Q₄ is independently selected from —CH— and —N— when Q₄ is attached to only single bonds, or Q₄ is ═CH— or ═N— when Q₄ is attached to a double bond.

In some embodiments, L comprises a structure selected from the group consisting of:

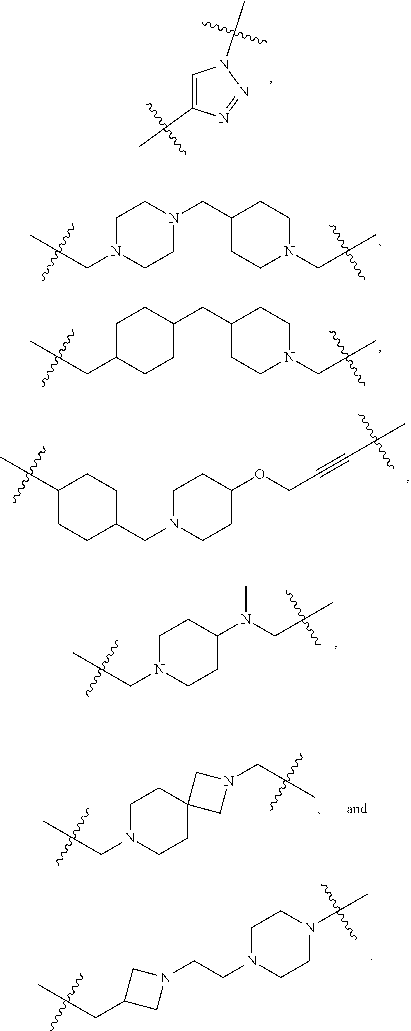

The compound of any one of claims 1-80, wherein L comprises a structure selected from the group consisting of:

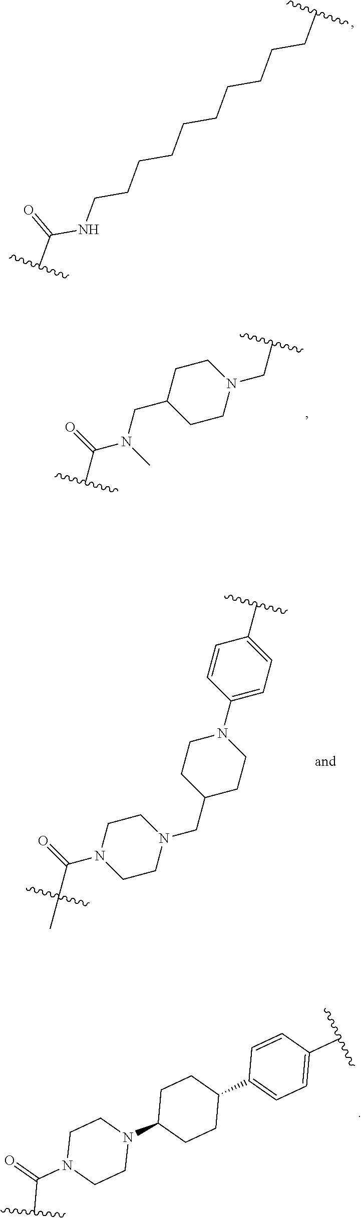 [SMILES: *CCCCCCCCCCNC(*)=O ; *CN1CCC(CN(C)C(*)=O)CC1 ; *C(=O)N1CCN(CC2CCN(c3ccc(*)cc3)CC2)CC1 ; *C(=O)N1CCN([C@H]2CC[C@H](c3ccc(*)cc3)CC2)CC1]

In some embodiments,

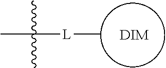

is selected from the group consisting of is

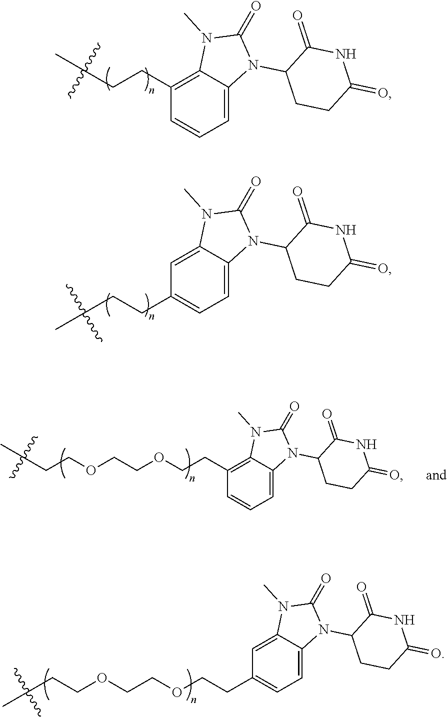

In some embodiments, a provided compound or pharmaceutically acceptable salt thereof, is selected from compounds 1.1-1.8, shown below:

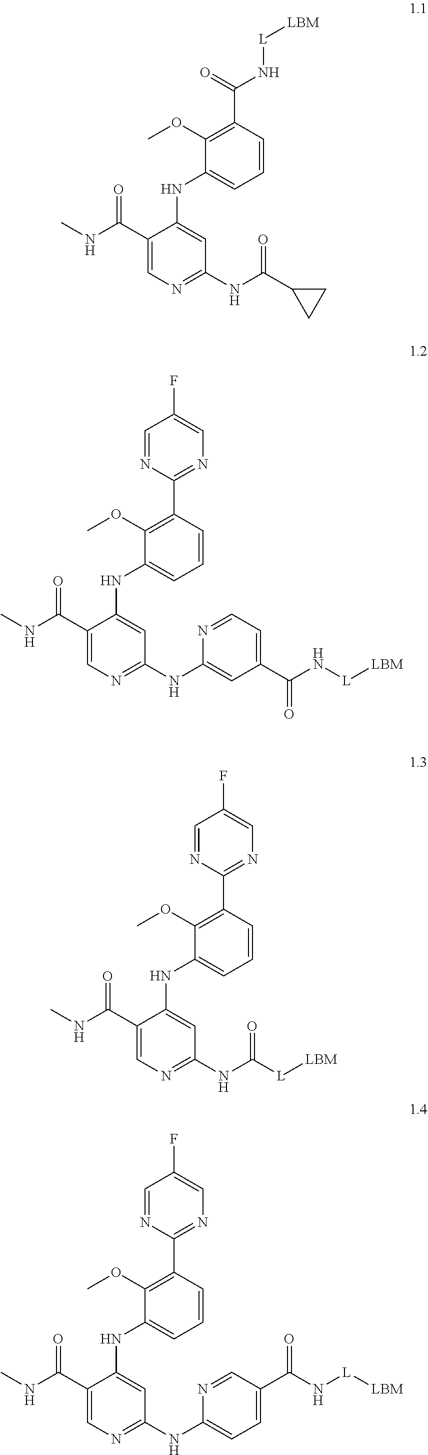

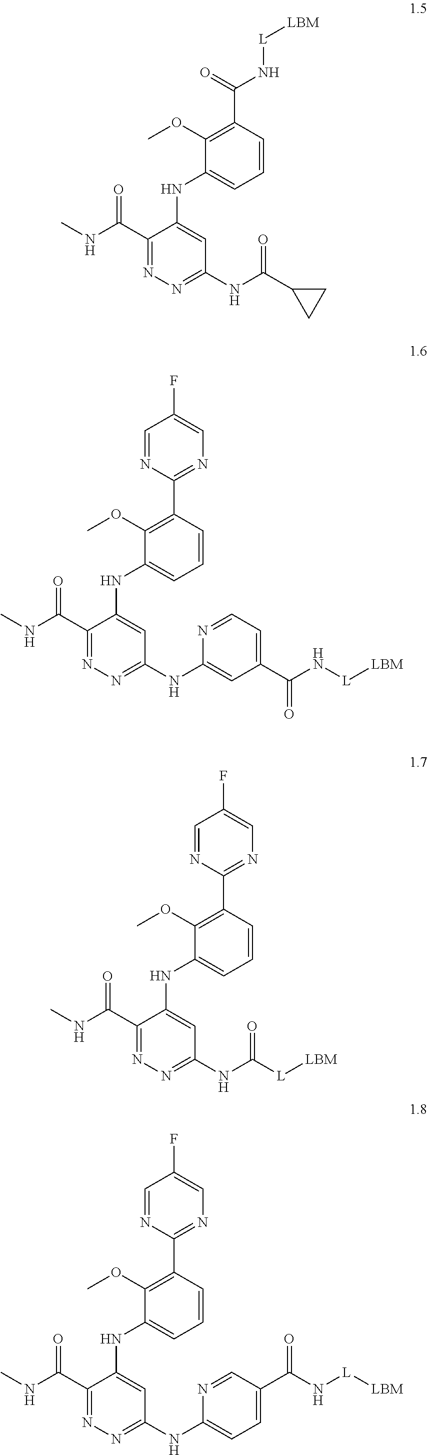

wherein LBM is selected from any of those in Table A below, and L is selected from any of those in Table B below.

In some embodiments, a provided compound, or pharmaceutically acceptable salt thereof, is selected from compounds 2.1-2.7, shown below:

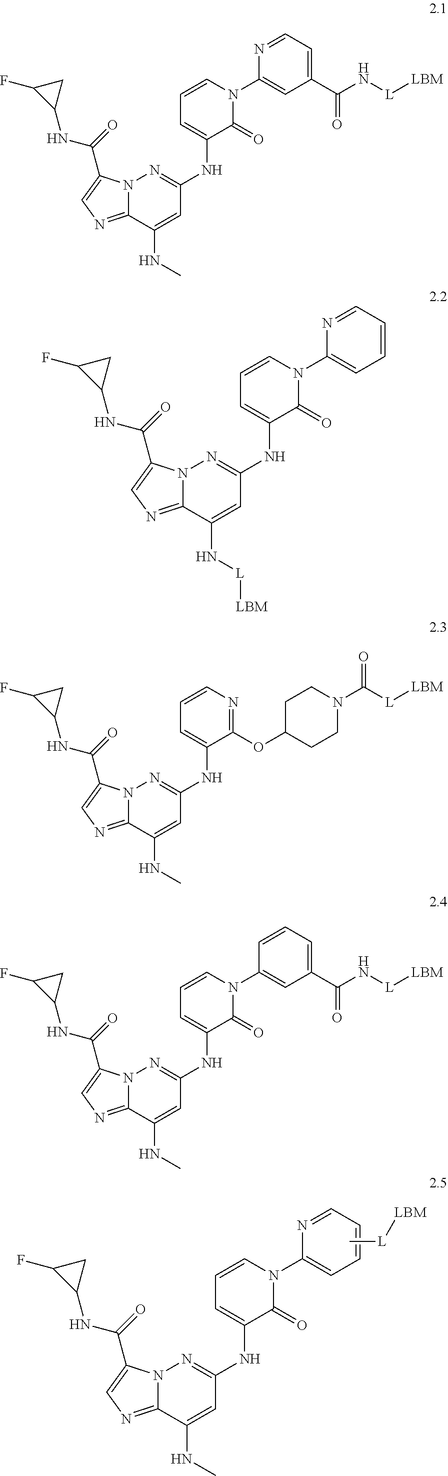

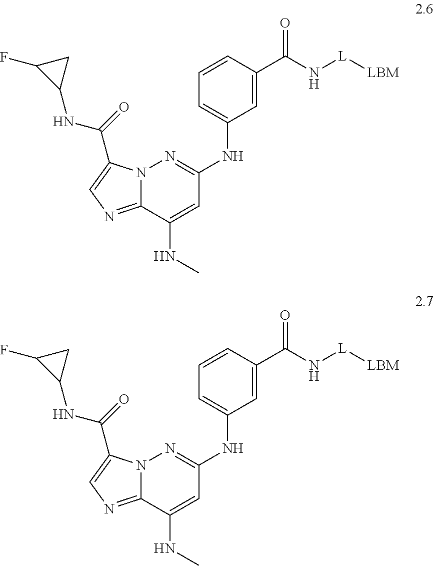

wherein LBM is selected from any of those in Table A below, and L is selected from any of those in Table B below.

In some embodiments, a provided compound, or pharmaceutically acceptable salt thereof, is selected from compounds 2.1A-2.7A, shown below:

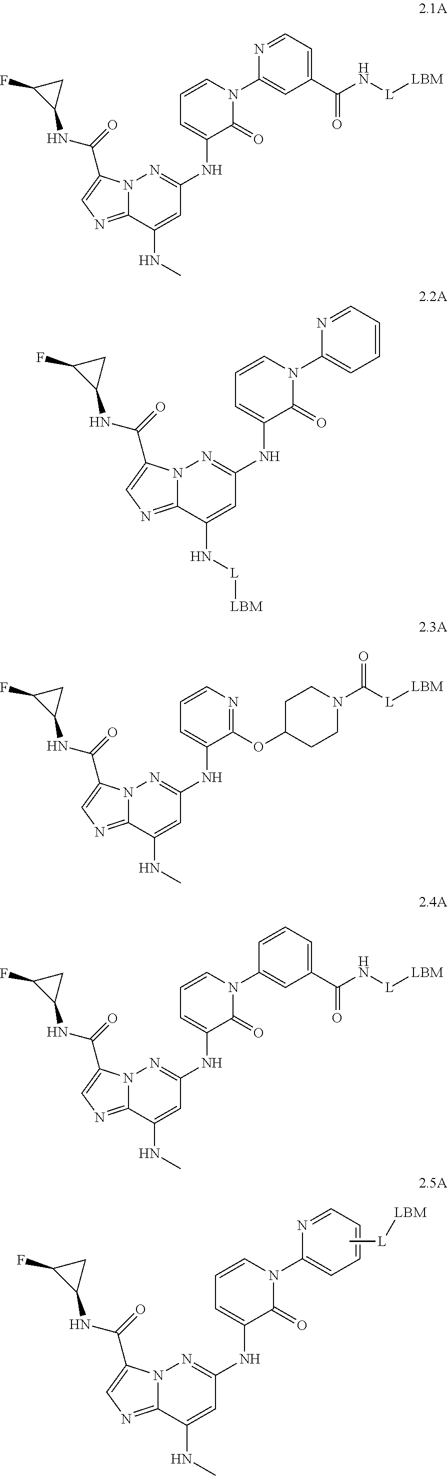

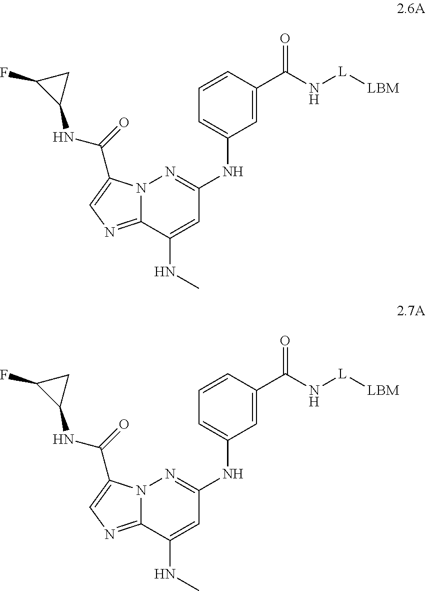

wherein LBM is selected from any of those in Table A below, and L is selected from any of those in Table B below.

TABLE A
Exemplified E3 Ligase Binding Moiety (LBM)

| | |
|---|---|
| 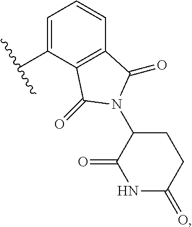 [SMILES: *c1cccc2c1C(=O)N(C1CCC(=O)NC1=O)C2=O] | (a) |
| 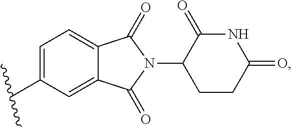 [SMILES: *c1ccc2c(c1)C(=O)N(C1CCC(=O)NC1=O)C2=O] | (b) |
| 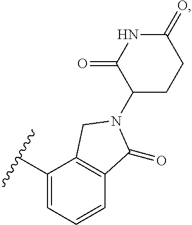 [SMILES: *c1cccc2c1CN(C1CCC(=O)NC1=O)C2=O] | (c) |
| 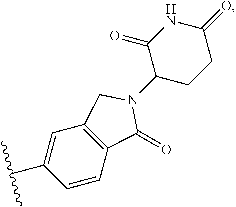 [SMILES: *c1ccc2c(c1)CN(C1CCC(=O)NC1=O)C2=O] | (d) |
| 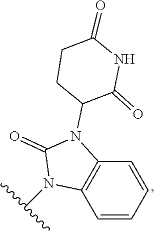 [SMILES: *n1c(=O)n(C2CCC(=O)NC2=O)c2ccccc21] | (e) |
| 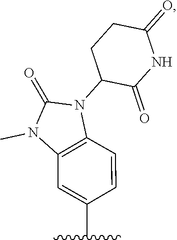 [SMILES: *c1ccc2c(c1)n(C)c(=O)n2C1CCC(=O)NC1=O] | (f) |
| 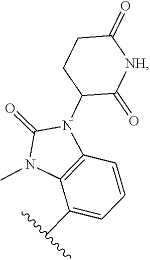 [SMILES: *c1cccc2c1n(C)c(=O)n2C1CCC(=O)NC1=O] | (g) |
| 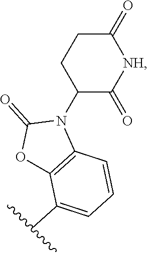 [SMILES: *c1cccc2c1oc(=O)n2C1CCC(=O)NC1=O] | (h) |
| 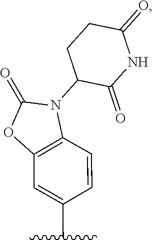 [SMILES: *c1ccc2c(c1)oc(=O)n2C1CCC(=O)NC1=O] | (i) |
| 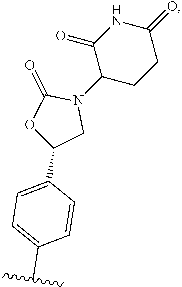 [SMILES: *c1ccc([C@H]2CN(C3CCC(=O)NC3=O)C(=O)O2)cc1] | (j) |
| 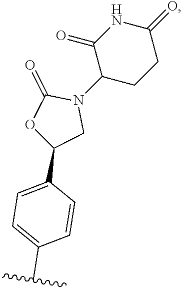 [SMILES: *c1ccc([C@@H]2CN(C3CCC(=O)NC3=O)C(=O)O2)cc1] | (k) |
| 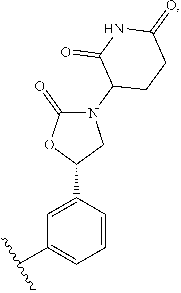 [SMILES: *c1cccc([C@H]2CN(C3CCC(=O)NC3=O)C(=O)O2)c1] | (l) |
| 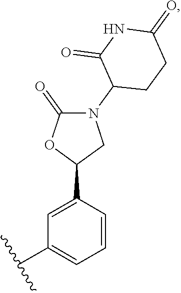 [SMILES: *c1cccc([C@@H]2CN(C3CCC(=O)NC3=O)C(=O)O2)c1] | (m) |
| 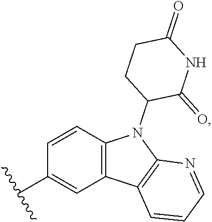 [SMILES: *c1ccc2c(c1)c1cccnc1n2C1CCC(=O)NC1=O] | (n) |
| 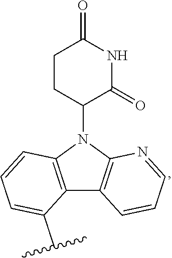 [SMILES: *c1cccc2c1c1cccnc1n2C1CCC(=O)NC1=O] | (o) |
| 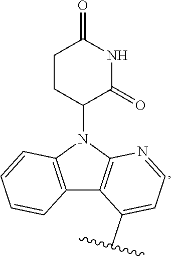 [SMILES: *c1ccnc2c1c1ccccc1n2C1CCC(=O)NC1=O] | (p) |
| 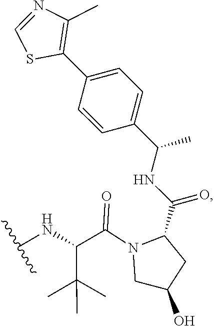 [SMILES: *N[C@H](C(=O)N1C[C@H](O)C[C@H]1C(=O)N[C@@H](C)c1ccc(-c2scnc2C)cc1)C(C)(C)C] | (q) |
| 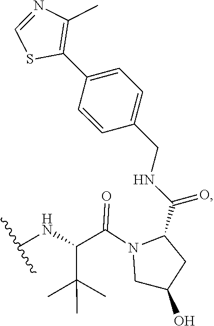 [SMILES: *N[C@H](C(=O)N1C[C@H](O)C[C@H]1C(=O)NCc1ccc(-c2scnc2C)cc1)C(C)(C)C] | (r) |
| 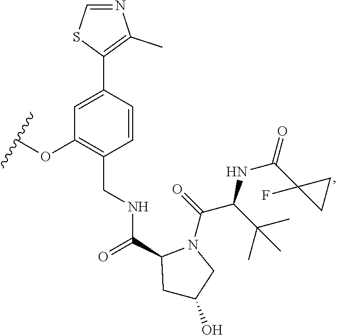 [SMILES: *Oc1cc(-c2scnc2C)ccc1CNC(=O)[C@@H]1C[C@@H](O)CN1C(=O)[C@@H](NC(=O)C1(F)CC1)C(C)(C)C] | (s) |
| 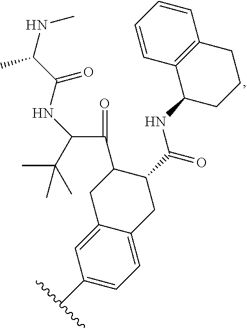 [SMILES: *c1ccc2c(c1)CC(C(=O)C(NC(=O)[C@H](C)NC)C(C)(C)C)[C@H](C(=O)N[C@@H]1CCCc3ccccc31)C2] | (t) |
| 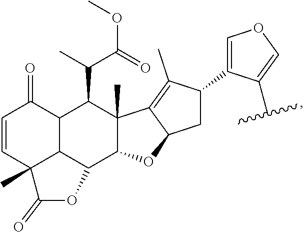 | (u) |
| 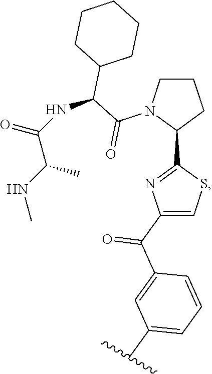 [SMILES: *c1cccc(C(=O)c2csc([C@@H]3CCCN3C(=O)[C@@H](NC(=O)[C@H](C)NC)C3CCCCC3)n2)c1] | (v) |
| 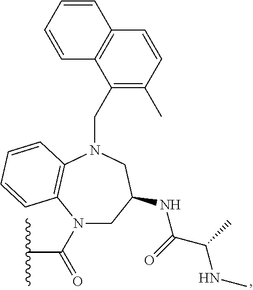 [SMILES: *C(=O)N1C[C@H](NC(=O)[C@H](C)NC)CN(Cc2c(C)ccc3ccccc23)c2ccccc21] | (w) |
| 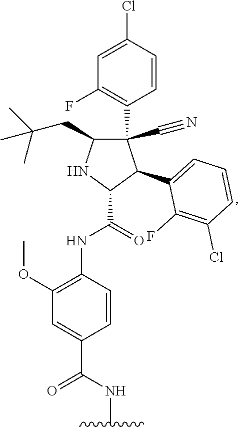 [SMILES: *NC(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1] | (x) |
| 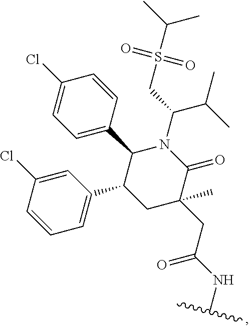 [SMILES: *NC(=O)C[C@@]1(C)C[C@H](c2cccc(Cl)c2)[C@@H](c2ccc(Cl)cc2)N([C@H](CS(=O)(=O)C(C)C)C(C)C)C1=O] | (y) |
| 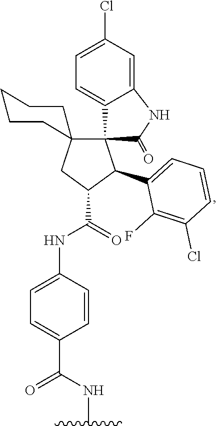 [SMILES: *NC(=O)c1ccc(NC(=O)[C@@H]2CC3(CCCCC3)[C@@]3(C(=O)Nc4cc(Cl)ccc43)[C@H]2c2cccc(Cl)c2F)cc1] | (z) |
| 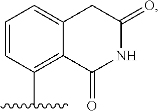 [SMILES: *c1cccc2c1C(=O)NC(=O)C2] | (aa) |
| 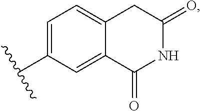 [SMILES: *c1ccc2c(c1)C(=O)NC(=O)C2] | (bb) |
| 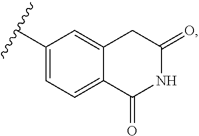 [SMILES: *c1ccc2c(c1)CC(=O)NC2=O] | (cc) |
| 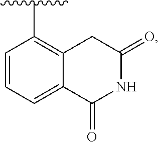 [SMILES: *c1cccc2c1CC(=O)NC2=O] | (dd) |
| 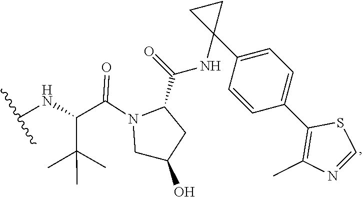 [SMILES: *N[C@H](C(=O)N1C[C@H](O)C[C@H]1C(=O)NC1(c2ccc(-c3scnc3C)cc2)CC1)C(C)(C)C] | (ee) |
| 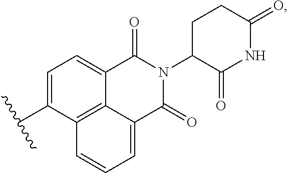 [SMILES: *c1ccc2c3c(cccc13)C(=O)N(C1CCC(=O)NC1=O)C2=O] | (ff) |
| 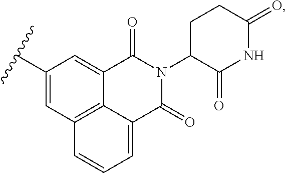 [SMILES: *c1cc2c3c(cccc3c1)C(=O)N(C1CCC(=O)NC1=O)C2=O] | (gg) |
| 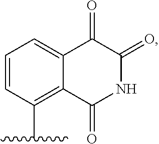 [SMILES: *c1cccc2c1C(=O)NC(=O)C2=O] | (hh) |
| 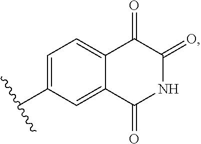 [SMILES: *c1ccc2c(c1)C(=O)NC(=O)C2=O] | (ii) |
| 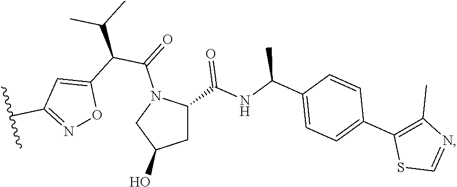 [SMILES: *c1cc([C@H](C(=O)N2C[C@H](O)C[C@H]2C(=O)N[C@@H](C)c2ccc(-c3scnc3C)cc2)C(C)C)on1] | (jj) |
| 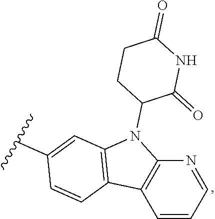 [SMILES: *c1ccc2c3cccnc3n(C3CCC(=O)NC3=O)c2c1] | (kk) |
| 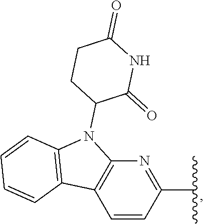 [SMILES: *c1ccc2c3ccccc3n(C3CCC(=O)NC3=O)c2n1] | (ll) |
| 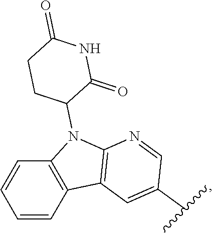 [SMILES: *c1cnc2c(c1)c1ccccc1n2C1CCC(=O)NC1=O] | (mm) |
| 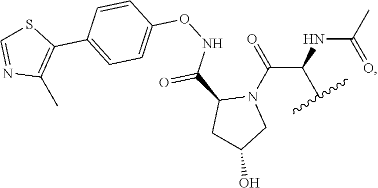 [SMILES: *[C@H](NC(C)=O)C(=O)N1C[C@H](O)C[C@H]1C(=O)NOc1ccc(-c2scnc2C)cc1] | (nn) |
| 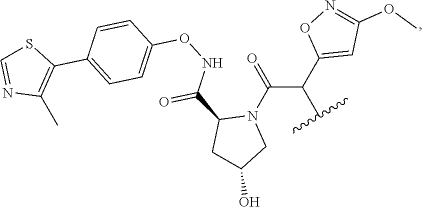 [SMILES: *C(C(=O)N1C[C@H](O)C[C@H]1C(=O)NOc1ccc(-c2scnc2C)cc1)c1cc(OC)no1] | (oo) |
| 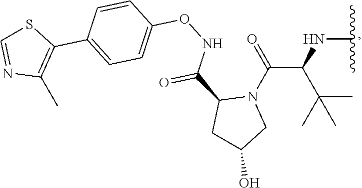 [SMILES: *N[C@H](C(=O)N1C[C@H](O)C[C@H]1C(=O)NOc1ccc(-c2scnc2C)cc1)C(C)(C)C] | (pp) |
| 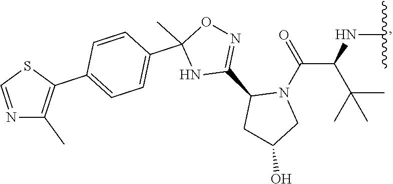 [SMILES: *N[C@H](C(=O)N1C[C@H](O)C[C@H]1C1=NOC(C)(c2ccc(-c3scnc3C)cc2)N1)C(C)(C)C] | (qq) |
| 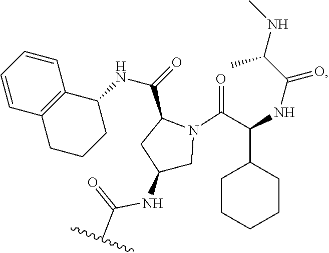 [SMILES: *C(=O)N[C@H]1C[C@@H](C(=O)N[C@@H]2CCCc3ccccc32)N(C(=O)[C@@H](NC(=O)[C@H](C)NC)C2CCCCC2)C1] | (rr) |
| 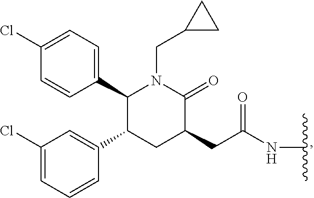 [SMILES: *NC(=O)C[C@H]1C[C@H](c2cccc(Cl)c2)[C@@H](c2ccc(Cl)cc2)N(CC2CC2)C1=O] | (ss) |
| 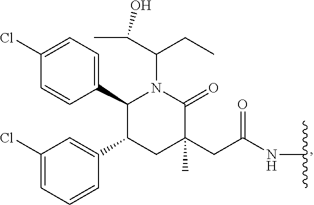 [SMILES: *NC(=O)C[C@@]1(C)C[C@H](c2cccc(Cl)c2)[C@@H](c2ccc(Cl)cc2)N(C(CC)[C@H](C)O)C1=O] | (tt) |
| 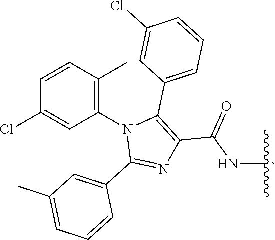 [SMILES: *NC(=O)c1nc(-c2cccc(C)c2)n(-c2cc(Cl)ccc2C)c1-c1cccc(Cl)c1] | (uu) |
| 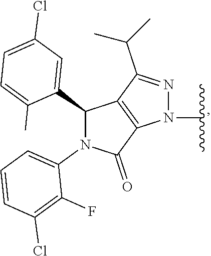 [SMILES: *n1nc(C(C)C)c2c1C(=O)N(c1cccc(Cl)c1F)[C@H]2c1cc(Cl)ccc1C] | (vv) |
| 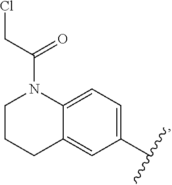 [SMILES: *c1ccc2c(c1)CCCN2C(=O)CCl] | (ww) |
| 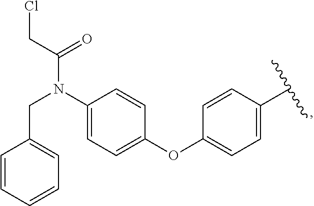 [SMILES: *c1ccc(Oc2ccc(N(Cc3ccccc3)C(=O)CCl)cc2)cc1] | (xx) |
| 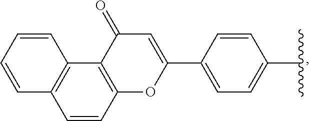 [SMILES: *c1ccc(-c2cc(=O)c3c(ccc4ccccc43)o2)cc1] | (yy) |
| 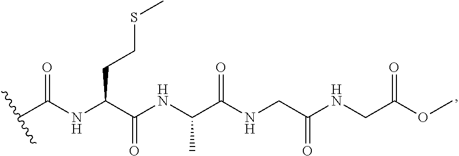 [SMILES: *C(=O)N[C@@H](CCSC)C(=O)N[C@@H](C)C(=O)NCC(=O)NCC(=O)OC] | (zz) |
| 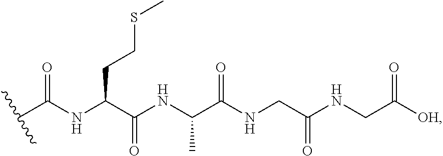 [SMILES: *C(=O)N[C@@H](CCSC)C(=O)N[C@@H](C)C(=O)NCC(=O)NCC(=O)O] | (aaa) |
| 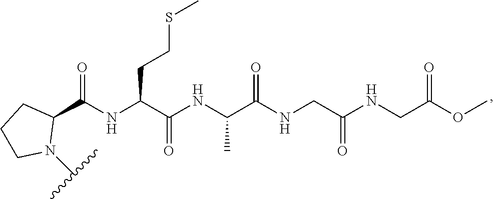 [SMILES: *N1CCC[C@H]1C(=O)N[C@@H](CCSC)C(=O)N[C@@H](C)C(=O)NCC(=O)NCC(=O)OC] | (bbb) |
| 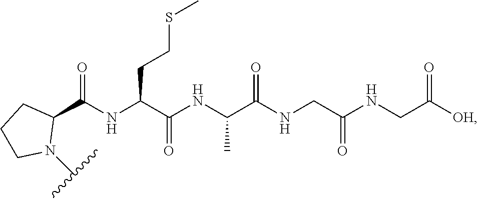 [SMILES: *N1CCC[C@H]1C(=O)N[C@@H](CCSC)C(=O)N[C@@H](C)C(=O)NCC(=O)NCC(=O)O] | (ccc) |
| 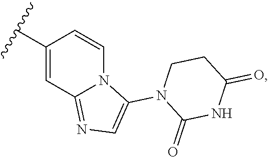 [SMILES: *c1ccn2c(N3CCC(=O)NC3=O)cnc2c1] | (ddd) |
| 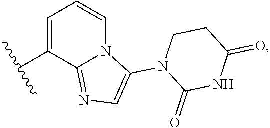 [SMILES: *c1cccn2c(N3CCC(=O)NC3=O)cnc12] | (eee) |
| 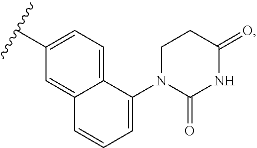 [SMILES: *c1ccc2c(N3CCC(=O)NC3=O)cccc2c1] | (fff) |
| 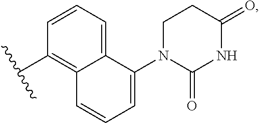 [SMILES: *c1cccc2c(N3CCC(=O)NC3=O)cccc12] | (ggg) |
| 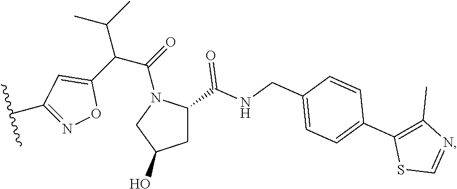 [SMILES: *c1cc(C(C(=O)N2C[C@H](O)C[C@H]2C(=O)NCc2ccc(-c3scnc3C)cc2)C(C)C)on1] | (hhh) |
| 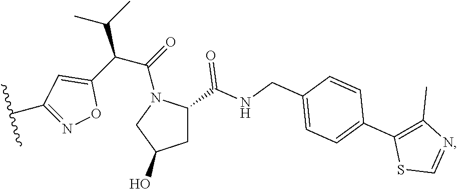 [SMILES: *c1cc([C@H](C(=O)N2C[C@H](O)C[C@H]2C(=O)NCc2ccc(-c3scnc3C)cc2)C(C)C)on1] | (iii) |
| 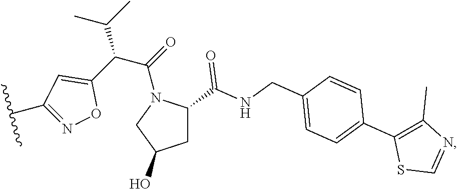 [SMILES: *c1cc([C@@H](C(=O)N2C[C@H](O)C[C@H]2C(=O)NCc2ccc(-c3scnc3C)cc2)C(C)C)on1] | (jjj) |
| 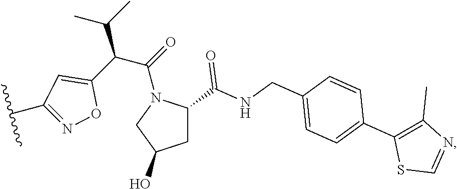 [SMILES: *c1cc([C@H](C(=O)N2C[C@H](O)C[C@H]2C(=O)NCc2ccc(-c3scnc3C)cc2)C(C)C)on1] | (kkk) |
| 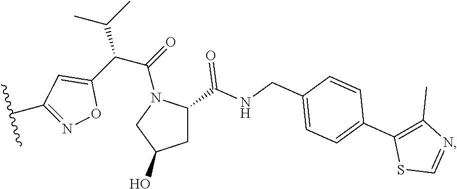 [SMILES: *c1cc([C@@H](C(=O)N2C[C@H](O)C[C@H]2C(=O)NCc2ccc(-c3scnc3C)cc2)C(C)C)on1] | (lll) |
| 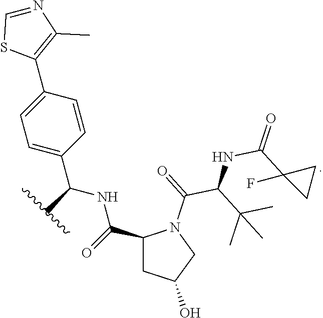 [SMILES: *[C@H](NC(=O)[C@@H]1C[C@@H](O)CN1C(=O)[C@@H](NC(=O)C1(F)CC1)C(C)(C)C)c1ccc(-c2scnc2C)cc1] | (mmm) |

TABLE B
Exemplified Linkers (L)

| | |
|---|---|
| 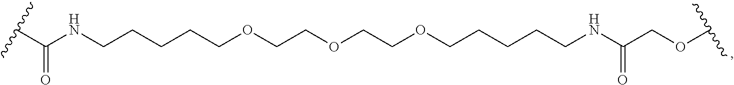 [SMILES: *OCC(=O)NCCCCCOCCOCCOCCCCCNC(*)=O] | (1) |
| 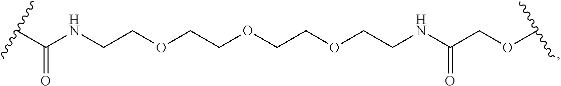 [SMILES: *OCC(=O)NCCOCCOCCOCCNC(*)=O] | (2) |
| 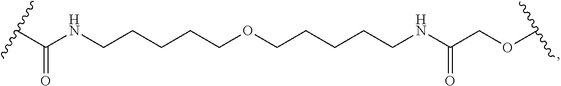 [SMILES: *OCC(=O)NCCCCCOCCCCCNC(*)=O] | (3) |
| 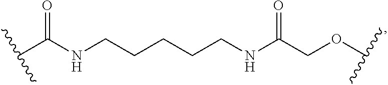 [SMILES: *OCC(=O)NCCCCCNC(*)=O] | (4) |
| 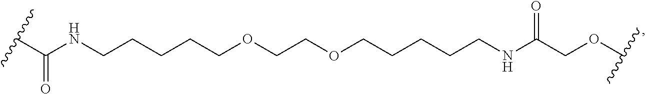 [SMILES: *OCC(=O)NCCCCCOCCOCCCCCNC(*)=O] | (5) |
| 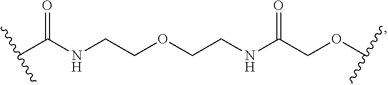 [SMILES: *OCC(=O)NCCOCCNC(*)=O] | (6) |
| 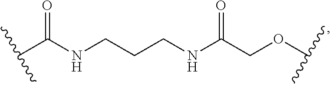 [SMILES: *OCC(=O)NCCCNC(*)=O] | (7) |
| 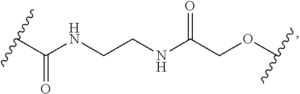 [SMILES: *OCC(=O)NCCNC(*)=O] | (8) |
| 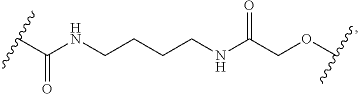 [SMILES: *OCC(=O)NCCCCNC(*)=O] | (9) |
| 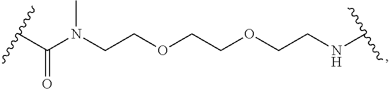 [SMILES: *NCCOCCOCCN(C)C(*)=O] | (10) |
| 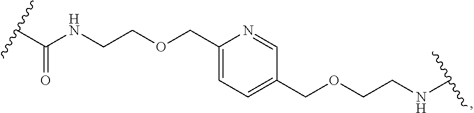 [SMILES: *NCCOCc1ccc(COCCNC(*)=O)nc1] | (11) |
| 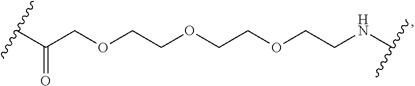 [SMILES: *NCCOCCOCCOCC(*)=O] | (12) |
| 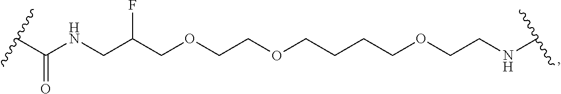 [SMILES: *NCCOCCCCOCCOCC(F)CNC(*)=O] | (13) |
| 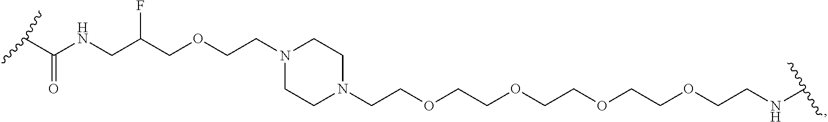 [SMILES: *NCCOCCOCCOCCOCCN1CCN(CCOCC(F)CNC(*)=O)CC1] | (14) |
| 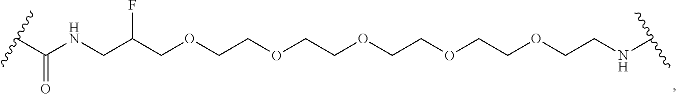 [SMILES: *NCCOCCOCCOCCOCCOCC(F)CNC(*)=O] | (15) |
| 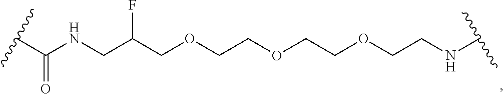 [SMILES: *NCCOCCOCCOCC(F)CNC(*)=O] | (16) |
| 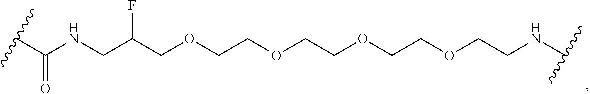 [SMILES: *NCCOCCOCCOCCOCC(F)CNC(*)=O] | (17) |
| 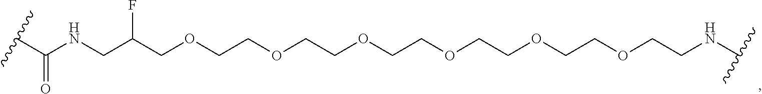 [SMILES: *NCCOCCOCCOCCOCCOCCOCC(F)CNC(*)=O] | (18) |
| 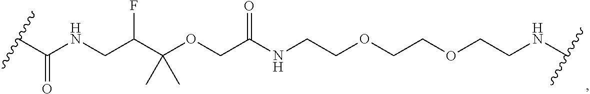 [SMILES: *NCCOCCOCCNC(=O)COC(C)(C)C(F)CNC(*)=O] | (19) |
| 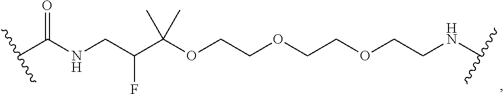 [SMILES: *NCCOCCOCCOC(C)(C)C(F)CNC(*)=O] | (20) |
| 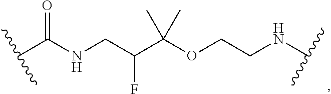 [SMILES: *NCCOC(C)(C)C(F)CNC(*)=O] | (21) |
| 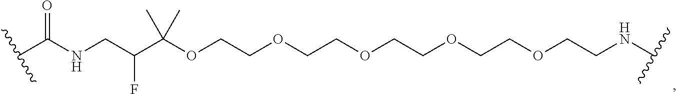 [SMILES: *NCCOCCOCCOCCOCCOC(C)(C)C(F)CNC(*)=O] | (22) |
| 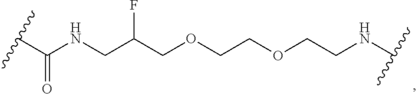 [SMILES: *NCCOCCOCC(F)CNC(*)=O] | (23) |
| 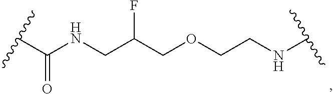 [SMILES: *NCCOCC(F)CNC(*)=O] | (24) |
| 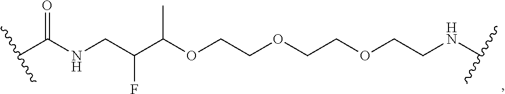 [SMILES: *NCCOCCOCCOC(C)C(F)CNC(*)=O] | (25) |
| 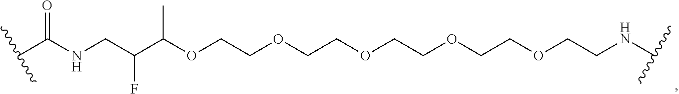 [SMILES: *NCCOCCOCCOCCOCCOC(C)C(F)CNC(*)=O] | (26) |
| 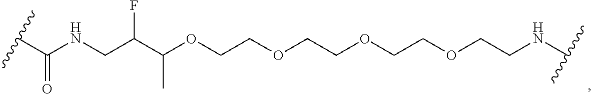 [SMILES: *NCCOCCOCCOCCOC(C)C(F)CNC(*)=O] | (27) |
| 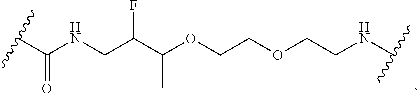 [SMILES: *NCCOCCOC(C)C(F)CNC(*)=O] | (28) |
| 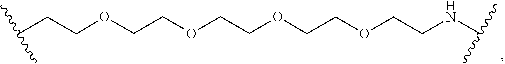 [SMILES: *CCOCCOCCOCCOCCN*] | (29) |
| 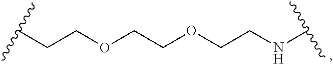 [SMILES: *CCOCCOCCN*] | (30) |
| 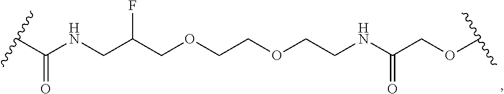 [SMILES: *OCC(=O)NCCOCCOCC(F)CNC(*)=O] | (31) |
| 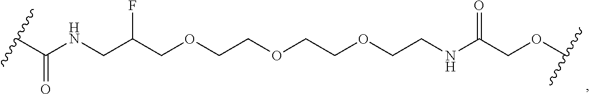 [SMILES: *OCC(=O)NCCOCCOCCOCC(F)CNC(*)=O] | (32) |
| 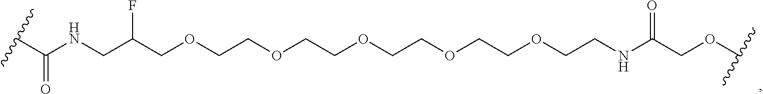 [SMILES: *OCC(=O)NCCOCCOCCOCCOCCOCC(F)CNC(*)=O] | (33) |
| 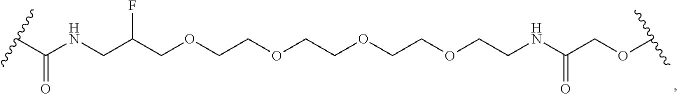 [SMILES: *OCC(=O)NCCOCCOCCOCCOCC(F)CNC(*)=O] | (34) |
| 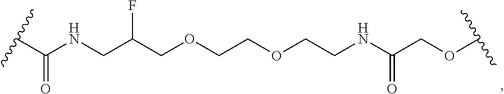 [SMILES: *OCC(=O)NCCOCCOCC(F)CNC(*)=O] | (35) |
| 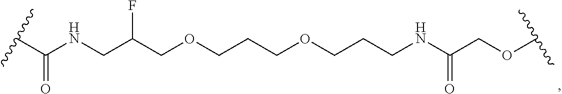 [SMILES: *OCC(=O)NCCCOCCCOCC(F)CNC(*)=O] | (36) |
| 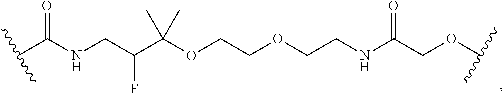 [SMILES: *OCC(=O)NCCOCCOC(C)(C)C(F)CNC(*)=O] | (37) |
| 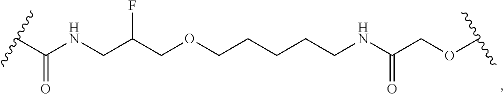 [SMILES: *OCC(=O)NCCCCCOCC(F)CNC(*)=O] | (38) |
| 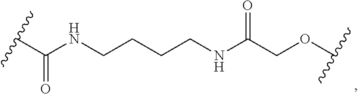 [SMILES: *OCC(=O)NCCCCNC(*)=O] | (39) |
| 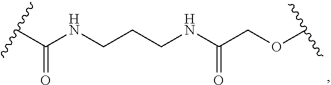 [SMILES: *OCC(=O)NCCCNC(*)=O] | (40) |
| 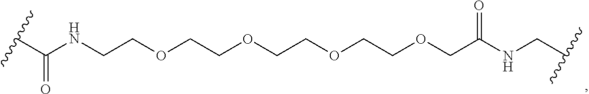 [SMILES: *CNC(=O)COCCOCCOCCOCCNC(*)=O] | (41) |
| 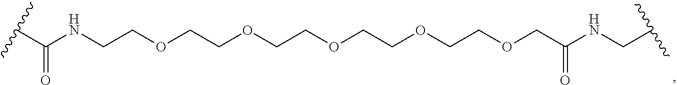 [SMILES: *CNC(=O)COCCOCCOCCOCCOCCNC(*)=O] | (42) |
| 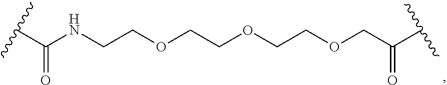 [SMILES: *C(=O)COCCOCCOCCNC(*)=O] | (43) |
| 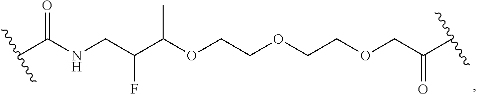 [SMILES: *C(=O)COCCOCCOC(C)C(F)CNC(*)=O] | (44) |
| 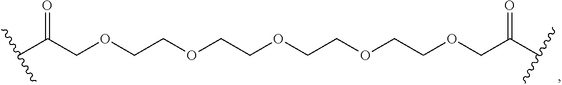 [SMILES: *C(=O)COCCOCCOCCOCCOCC(*)=O] | (45) |
| 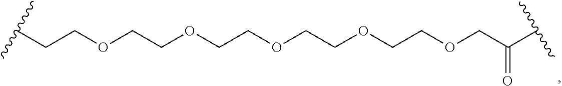 [SMILES: *CCOCCOCCOCCOCCOCC(*)=O] | (46) |
| 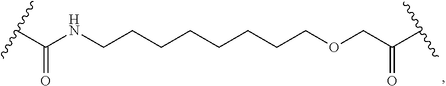 [SMILES: *C(=O)COCCCCCCCCNC(*)=O] | (47) |
| 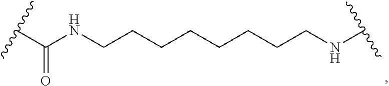 [SMILES: *NCCCCCCCCNC(*)=O] | (49) |
| 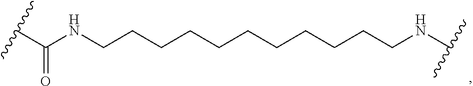 [SMILES: *NCCCCCCCCCCCNC(*)=O] | (50) |
| 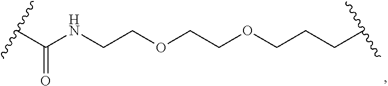 [SMILES: *CCCOCCOCCNC(*)=O] | (51 |
| 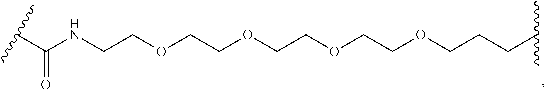 [SMILES: *CCCOCCOCCOCCOCCNC(*)=O] | (52) |
| 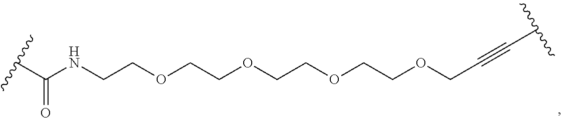 [SMILES: *C#CCOCCOCCOCCOCCNC(*)=O] | (53) |
| 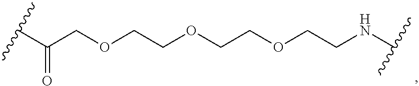 [SMILES: *NCCOCCOCCOCC(*)=O] | (54) |
| 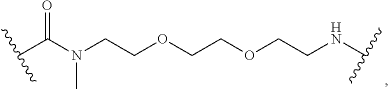 [SMILES: *NCCOCCOCCN(C)C(*)=O] | (55) |
| 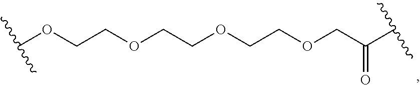 [SMILES: *OCCOCCOCCOCC(*)=O] | (56) |
| 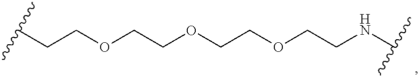 [SMILES: *CCOCCOCCOCCN*] | (57) |
| 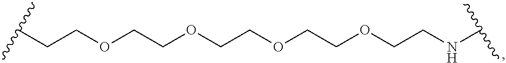 [SMILES: *CCOCCOCCOCCOCCN*] | (58) |
| 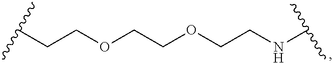 [SMILES: *CCOCCOCCN*] | (59) |
| 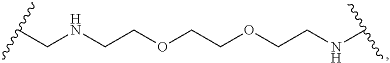 [SMILES: *CNCCOCCOCCN*] | (60) |
| 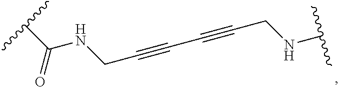 [SMILES: *NCC#CC#CCNC(*)=O] | (61) |
| 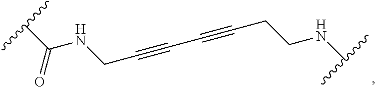 [SMILES: *NCCC#CC#CCNC(*)=O] | (62) |
| 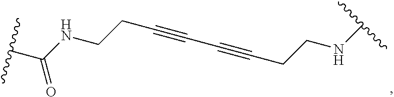 [SMILES: *NCCC#CC#CCCNC(*)=O] | (63) |
| 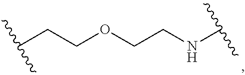 [SMILES: *CCOCCN*] | (64) |
| 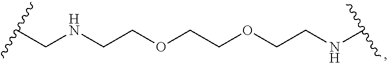 [SMILES: *CNCCOCCOCCN*] | (65) |
| 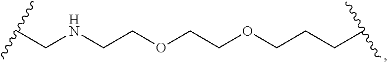 [SMILES: *CCCOCCOCCNC*] | (66) |
| 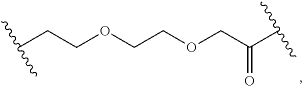 [SMILES: *CCOCCOCC(*)=O] | (67) |
| 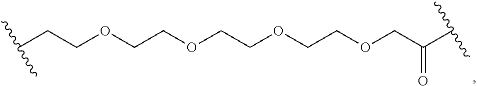 [SMILES: *CCOCCOCCOCCOCC(*)=O] | (68) |
| 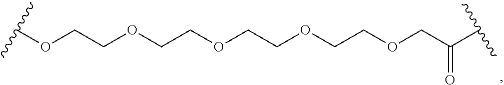 [SMILES: *OCCOCCOCCOCCOCC(*)=O] | (69) |
| 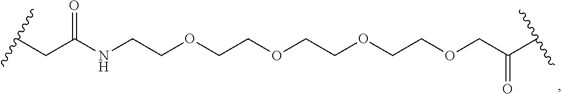 [SMILES: *CC(=O)NCCOCCOCCOCCOCC(*)=O] | (70) |
| 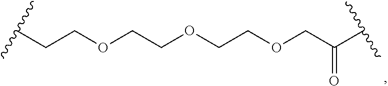 [SMILES: *CCOCCOCCOCC(*)=O] | (71) |
| 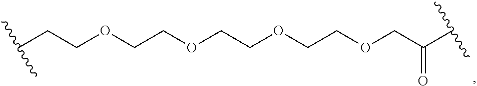 [SMILES: *CCOCCOCCOCCOCC(*)=O] | (72) |
| 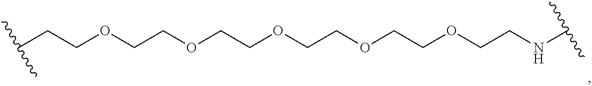 [SMILES: *CCOCCOCCOCCOCCOCCN*] | (73) |
| 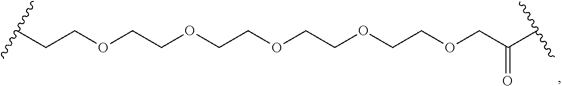 [SMILES: *CCOCCOCCOCCOCCOCC(*)=O] | (74) |
| 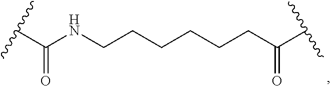 [SMILES: *C(=O)CCCCCCNC(*)=O] | (75) |
| 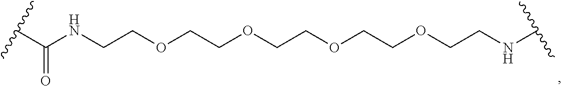 [SMILES: *NCCOCCOCCOCCOCCNC(*)=O] | (76) |
| 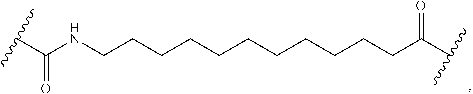 [SMILES: *C(=O)CCCCCCCCCCCNC(*)=O] | (77) |
| 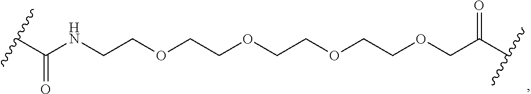 [SMILES: *C(=O)COCCOCCOCCOCCNC(*)=O] | (78) |
| 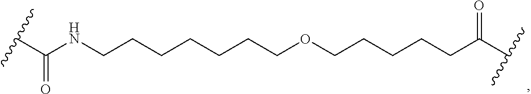 [SMILES: *C(=O)CCCCCOCCCCCCCNC(*)=O] | (79) |
| 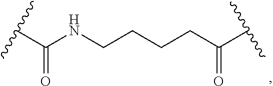 [SMILES: *C(=O)CCCCNC(*)=O] | (80) |
| 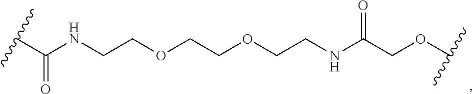 [SMILES: *OCC(=O)NCCOCCOCCNC(*)=O] | (81) |
| 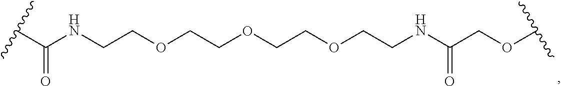 [SMILES: *OCC(=O)NCCOCCOCCOCCNC(*)=O] | (82) |
| 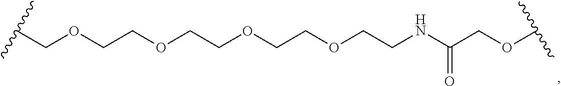 [SMILES: *COCCOCCOCCOCCNC(=O)CO*] | (83) |
| 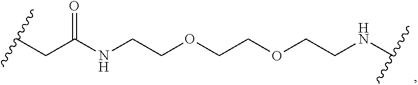 [SMILES: *CC(=O)NCCOCCOCCN*] | (84) |
| 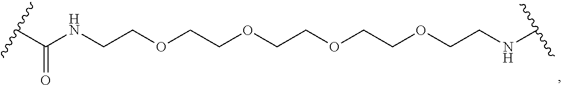 [SMILES: *NCCOCCOCCOCCOCCNC(*)=O] | (85) |
| 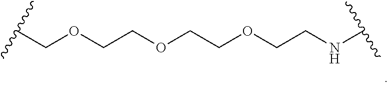 [SMILES: *COCCOCCOCCN*] | (86) |
| 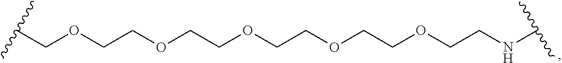 [SMILES: *COCCOCCOCCOCCOCCN*] | (87) |
| 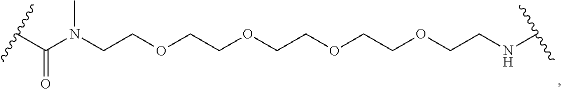 [SMILES: *NCCOCCOCCOCCOCCN(C)C(*)=O] | (88) |
| 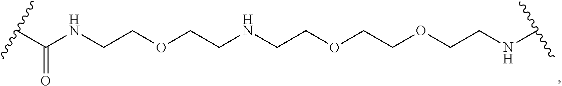 [SMILES: *NCCOCCOCCNCCOCCNC(*)=O] | (89) |
| 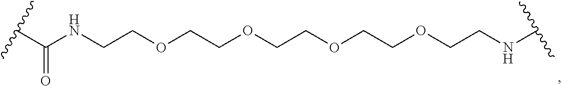 [SMILES: *NCCOCCOCCOCCOCCNC(*)=O] | (90) |
| 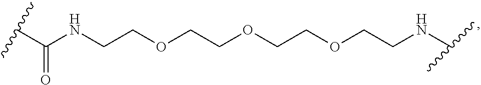 [SMILES: *NCCOCCOCCOCCNC(*)=O] | (91) |
| 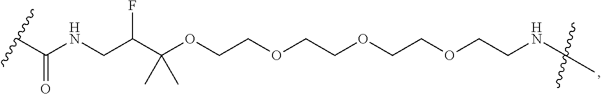 [SMILES: *NCCOCCOCCOCCOC(C)(C)C(F)CNC(*)=O] | (92) |
| 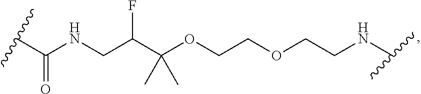 [SMILES: *NCCOCCOC(C)(C)C(F)CNC(*)=O] | (93) |
| 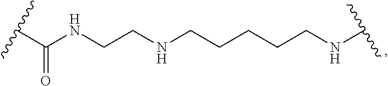 [SMILES: *NCCCCCNCCNC(*)=O] | (94) |
| 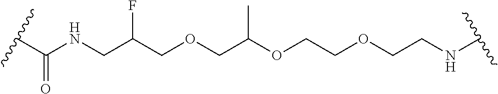 [SMILES: *NCCOCCOC(C)COCC(F)CNC(*)=O] | (95) |
| 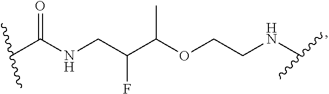 [SMILES: *NCCOC(C)C(F)CNC(*)=O] | (96) |
| 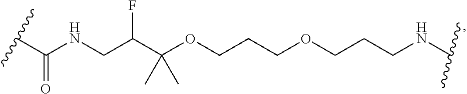 [SMILES: *NCCCOCCCOC(C)(C)C(F)CNC(*)=O] | (97) |
| 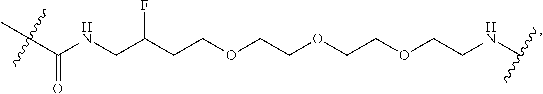 [SMILES: *NCCOCCOCCOCCC(F)CNC(*)=O] | (98) |
| 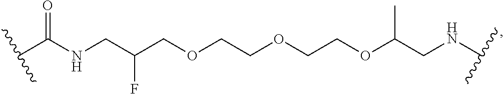 [SMILES: *NCC(C)OCCOCCOCC(F)CNC(*)=O] | (99) |
| 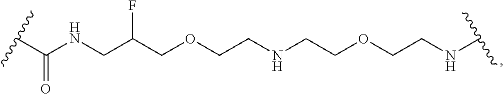 [SMILES: *NCCOCCNCCOCC(F)CNC(*)=O] | (100) |
| 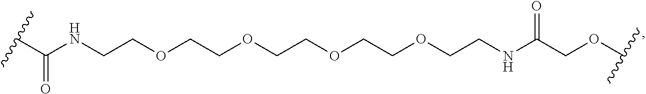 [SMILES: *OCC(=O)NCCOCCOCCOCCOCCNC(*)=O] | (101) |
| 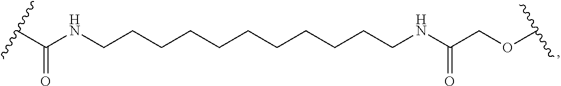 [SMILES: *OCC(=O)NCCCCCCCCCCCNC(*)=O] | (102) |
| 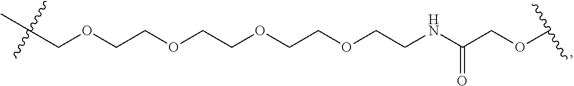 [SMILES: *COCCOCCOCCOCCNC(=O)CO*] | (103) |
| 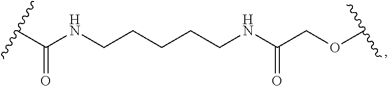 [SMILES: *OCC(=O)NCCCCCNC(*)=O] | (104) |
| 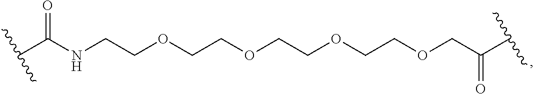 [SMILES: *C(=O)COCCOCCOCCOCCNC(*)=O] | (105) |
| 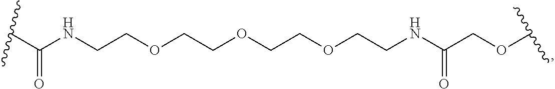 [SMILES: *OCC(=O)NCCOCCOCCOCCNC(*)=O] | (106) |
| 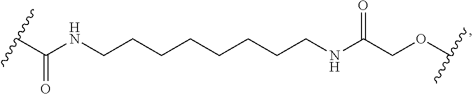 [SMILES: *OCC(=O)NCCCCCCCCNC(*)=O] | (107) |
| 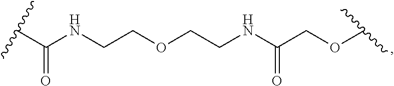 [SMILES: *OCC(=O)NCCOCCNC(*)=O] | (108) |
| 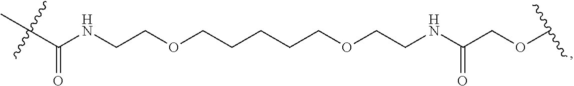 [SMILES: *OCC(=O)NCCOCCCCCOCCNC(*)=O] | (109) |
| 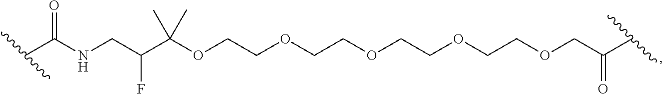 [SMILES: *C(=O)COCCOCCOCCOCCOC(C)(C)C(F)CNC(*)=O] | (110) |
| 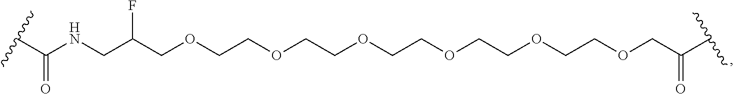 [SMILES: *C(=O)COCCOCCOCCOCCOCCOCC(F)CNC(*)=O] | (111) |
| 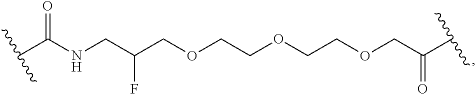 [SMILES: *C(=O)COCCOCCOCC(F)CNC(*)=O] | (112) |
| 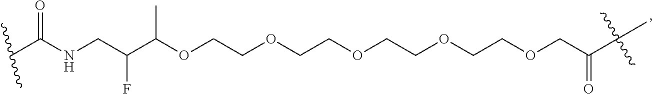 [SMILES: *C(=O)COCCOCCOCCOCCOC(C)C(F)CNC(*)=O] | (113) |
| 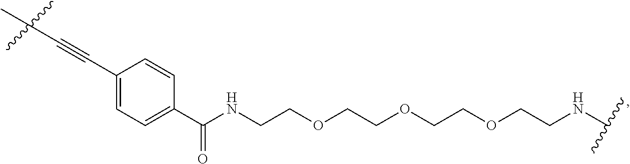 [SMILES: *C#Cc1ccc(C(=O)NCCOCCOCCOCCN*)cc1] | (114) |
| 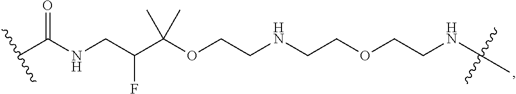 [SMILES: *NCCOCCNCCOC(C)(C)C(F)CNC(*)=O] | (115) |
| 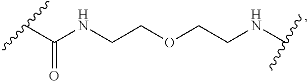 [SMILES: *NCCOCCNC(*)=O] | (116) |
| 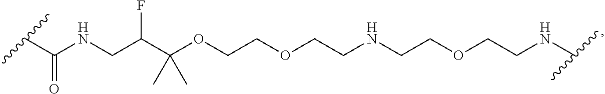 [SMILES: *NCCOCCNCCOCCOC(C)(C)C(F)CNC(*)=O] | (117) |
| 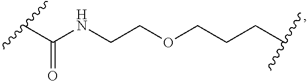 [SMILES: *CCCOCCNC(*)=O] | (118) |
| 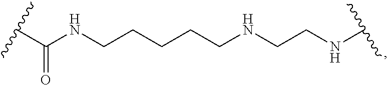 [SMILES: *NCCNCCCCCNC(*)=O] | |
| 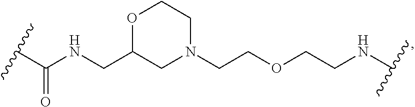 [SMILES: *NCCOCCN1CCOC(CNC(*)=O)C1] | (119) |
| 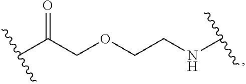 [SMILES: *NCCOCC(*)=O] | |
| 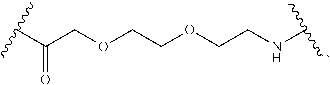 [SMILES: *NCCOCCOCC(*)=O] | (120) |
| 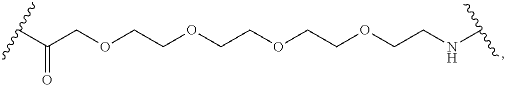 [SMILES: *NCCOCCOCCOCCOCC(*)=O] | (121) |
| 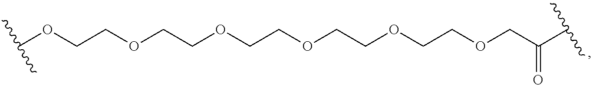 [SMILES: *OCCOCCOCCOCCOCCOCC(*)=O] | (122) |
| 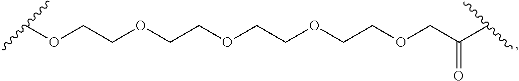 [SMILES: *OCCOCCOCCOCCOCC(*)=O] | (123) |
| 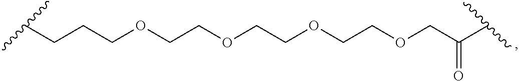 [SMILES: *CCCOCCOCCOCCOCC(*)=O] | (124) |
| 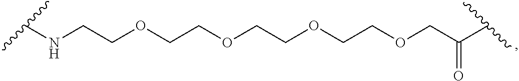 [SMILES: *NCCOCCOCCOCCOCC(*)=O] | (125) |
| 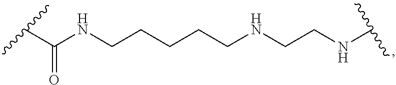 [SMILES: *NCCNCCCCCNC(*)=O] | (126) |
| 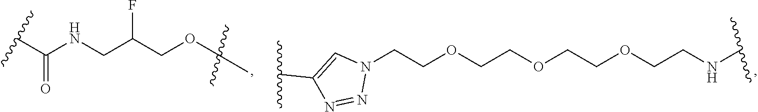 [SMILES: *OCC(F)CNC(*)=O ; *NCCOCCOCCOCCn1cc(*)nn1] | (127) |
| 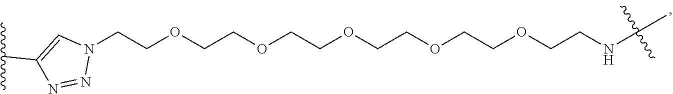 [SMILES: *NCCOCCOCCOCCOCCOCCn1cc(*)nn1] | (128) |
| 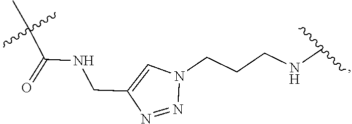 [SMILES: *NCCCn1cc(CNC(*)=O)nn1] | (129) |
| 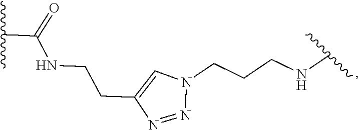 [SMILES: *NCCCn1cc(CCNC(*)=O)nn1] | (130) |
| 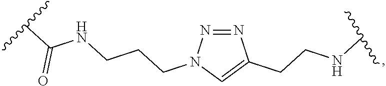 [SMILES: *NCCc1cn(CCCNC(*)=O)nn1] | (131) |
| 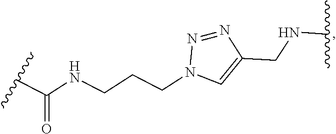 [SMILES: *NCc1cn(CCCNC(*)=O)nn1] | (132) |
| 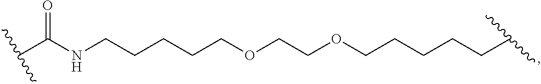 [SMILES: *CCCCCOCCOCCCCCNC(*)=O] | (133) |
| 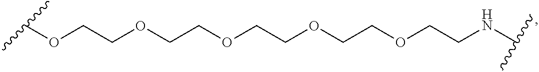 [SMILES: *NCCOCCOCCOCCOCCO*] | (134) |
| 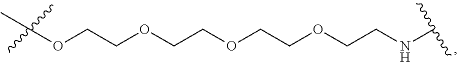 [SMILES: *NCCOCCOCCOCCO*] | (135) |
| 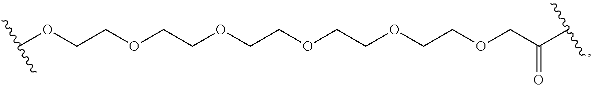 [SMILES: *OCCOCCOCCOCCOCCOCC(*)=O] | (136) |
| 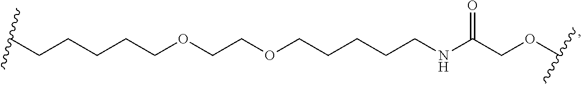 [SMILES: *CCCCCOCCOCCCCCNC(=O)CO*] | (137) |
| 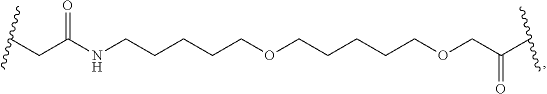 [SMILES: *CC(=O)NCCCCCOCCCCCOCC(*)=O] | (138) |
| 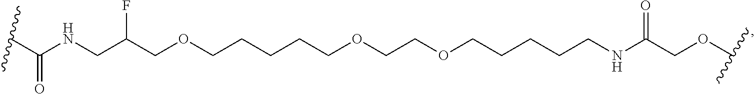 [SMILES: *OCC(=O)NCCCCCOCCOCCCCCOCC(F)CNC(*)=O] | (139) |
| 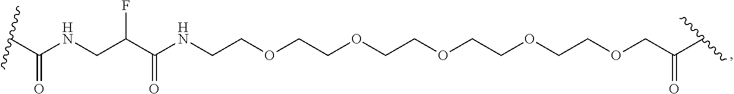 [SMILES: *C(=O)COCCOCCOCCOCCOCCNC(=O)C(F)CNC(*)=O] | (140) |
| 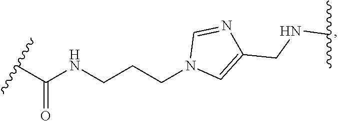 [SMILES: *NCc1cn(CCCNC(*)=O)cn1] | (141) |
| 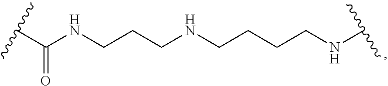 [SMILES: *NCCCCNCCCNC(*)=O] | (142) |
| 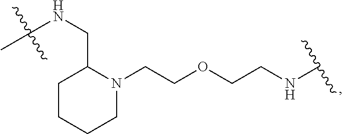 [SMILES: *NCCOCCN1CCCCC1CN*] | (143) |
| 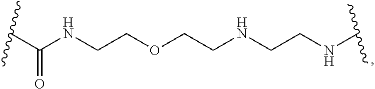 [SMILES: *NCCNCCOCCNC(*)=O] | (144) |
| 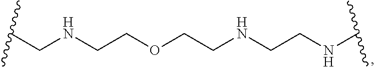 [SMILES: *CNCCOCCNCCN*] | (145) |
| 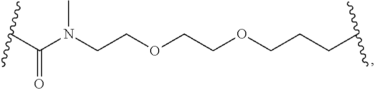 [SMILES: *CCCOCCOCCN(C)C(*)=O] | (146) |
| 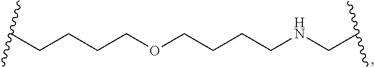 [SMILES: *CCCCOCCCCNC*] | (147) |
| 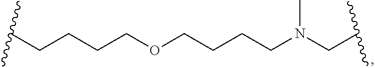 [SMILES: *CCCCOCCCCN(C)C*] | (148) |
| 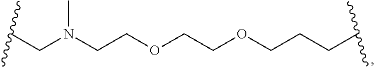 [SMILES: *CCCOCCOCCN(C)C*] | (149) |
| 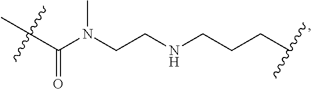 [SMILES: *CCCNCCN(C)C(*)=O] | (150) |
| 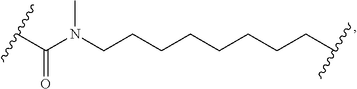 [SMILES: *CCCCCCCCN(C)C(*)=O] | (151) |
| 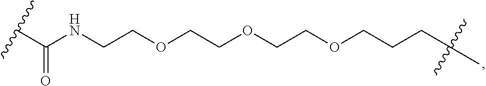 [SMILES: *CCCOCCOCCOCCNC(*)=O] | (152) |
| 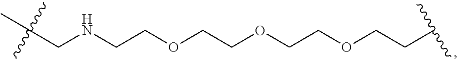 [SMILES: *CCOCCOCCOCCNC*] | (153) |
| 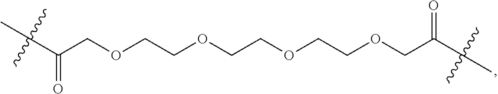 [SMILES: *C(=O)COCCOCCOCCOCC(*)=O] | (154) |
| 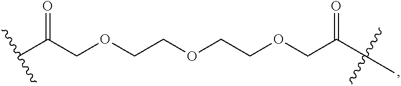 [SMILES: *C(=O)COCCOCCOCC(*)=O] | (155) |
| 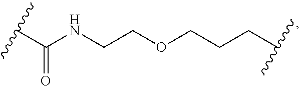 [SMILES: *CCCOCCNC(*)=O] | (156) |
| 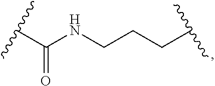 [SMILES: *CCCNC(*)=O] | (157) |
| 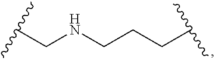 [SMILES: *CCCNC*] | (158) |
| 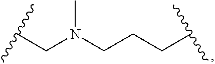 [SMILES: *CCCN(C)C*] | (159) |
| 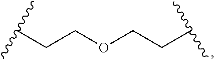 [SMILES: *CCOCC*] | (160) |
| 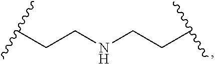 [SMILES: *CCNCC*] | (161) |
| 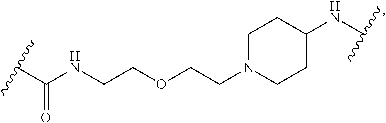 [SMILES: *NC1CCN(CCOCCNC(*)=O)CC1] | (162) |
| 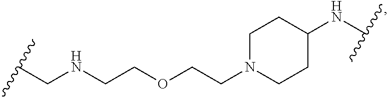 [SMILES: *CNCCOCCN1CCC(N*)CC1] | (163) |
| 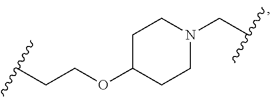 [SMILES: *CCOC1CCN(C*)CC1] | (164) |
| 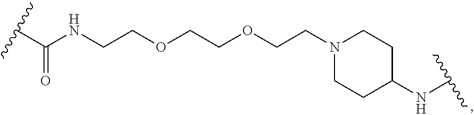 [SMILES: *NC1CCN(CCOCCOCCNC(*)=O)CC1] | (165) |
| 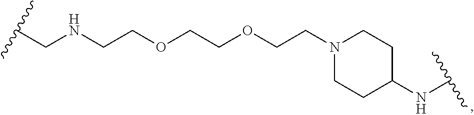 [SMILES: *CNCCOCCOCCN1CCC(N*)CC1] | (166) |
| 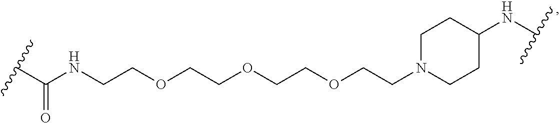 [SMILES: *NC1CCN(CCOCCOCCOCCNC(*)=O)CC1] | (167) |
| 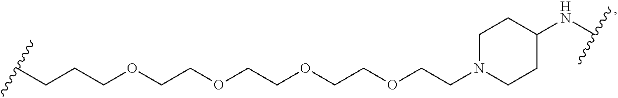 [SMILES: *CCCOCCOCCOCCOCCN1CCC(N*)CC1] | (168) |
| 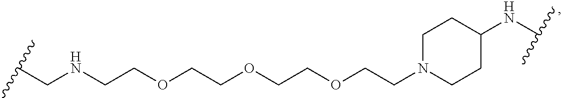 [SMILES: *CNCCOCCOCCOCCN1CCC(N*)CC1] | (169) |
| 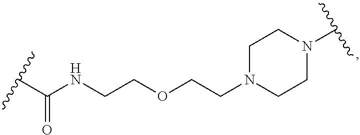 [SMILES: *C(=O)NCCOCCN1CCN(*)CC1] | (170) |
| 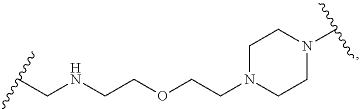 [SMILES: *CNCCOCCN1CCN(*)CC1] | (171) |
| 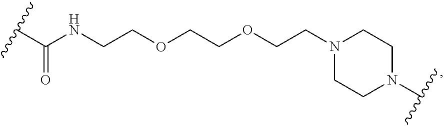 [SMILES: *C(=O)NCCOCCOCCN1CCN(*)CC1] | (172) |
| 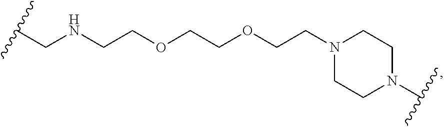 [SMILES: *CNCCOCCOCCN1CCN(*)CC1] | (173) |
| 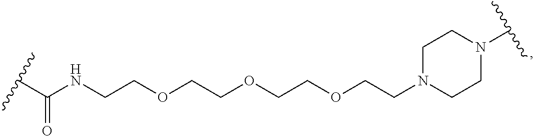 [SMILES: *C(=O)NCCOCCOCCOCCN1CCN(*)CC1] | (174) |
| 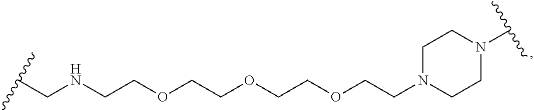 [SMILES: *CNCCOCCOCCOCCN1CCN(*)CC1] | (175) |
| 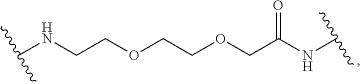 [SMILES: *NCCOCCOCC(=O)N*] | (176) |
| 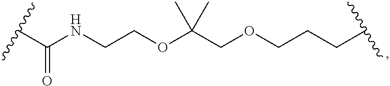 [SMILES: *CCCOCC(C)(C)OCCNC(*)=O] | (177) |
| 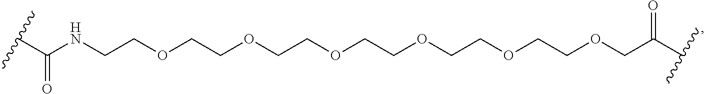 [SMILES: *C(=O)COCCOCCOCCOCCOCCOCCNC(*)=O] | (178) |
| 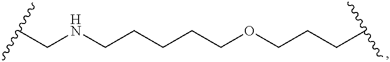 [SMILES: *CCCOCCCCCNC*] | (179) |
| 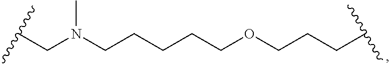 [SMILES: *CCCOCCCCCN(C)C*] | (180) |
| 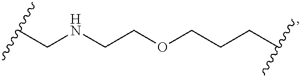 [SMILES: *CCCOCCNC*] | (181 ) |
| 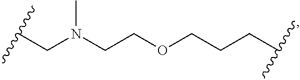 [SMILES: *CCCOCCN(C)C*] | (182) |
| 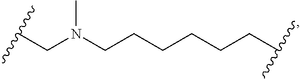 [SMILES: *CCCCCCN(C)C*] | (183) |
| 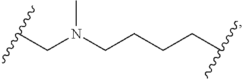 [SMILES: *CCCCN(C)C*] | (184) |
| 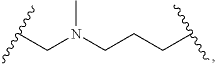 [SMILES: *CCCN(C)C*] | (185) |
| 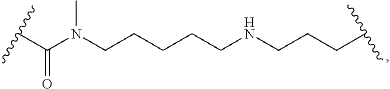 [SMILES: *CCCNCCCCCN(C)C(*)=O] | (186) |
| 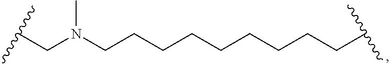 [SMILES: *CCCCCCCCCN(C)C*] | (187) |
| 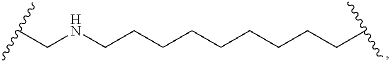 [SMILES: *CCCCCCCCCNC*] | (188) |
| 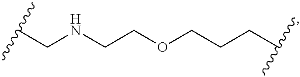 [SMILES: *CCCOCCNC*] | (189) |
| 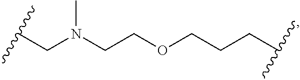 [SMILES: *CCCOCCN(C)C*] | (190) |
| 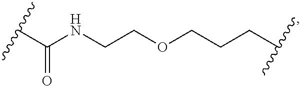 [SMILES: *CCCOCCNC(*)=O] | (191) |
| 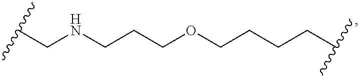 [SMILES: *CCCCOCCCNC*] | (192) |
| 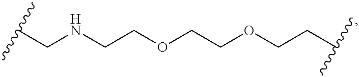 [SMILES: *CCOCCOCCNC*] | (193) |
| 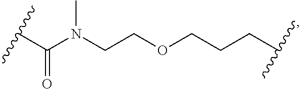 [SMILES: *CCCOCCN(C)C(*)=O] | (194) |
| 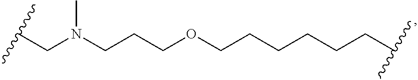 [SMILES: *CCCCCCOCCCN(C)C*] | (195) |
| 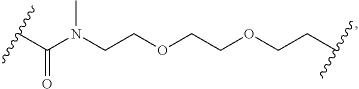 [SMILES: *CCOCCOCCN(C)C(*)=O] | (196) |
| 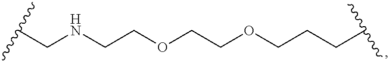 [SMILES: *CCCOCCOCCNC*] | (197) |
| 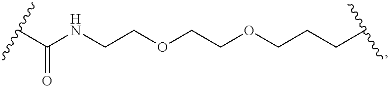 [SMILES: *CCCOCCOCCNC(*)=O] | (198) |
| 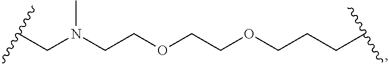 [SMILES: *CCCOCCOCCN(C)C*] | (199) |
| 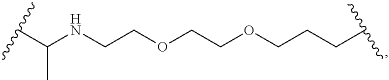 [SMILES: *CCCOCCOCCNC(*)C] | (200) |
| 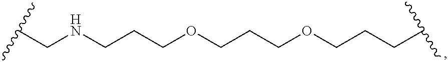 [SMILES: *CCCOCCCOCCCNC*] | (201) |
| 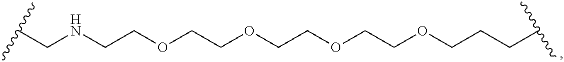 [SMILES: *CCCOCCOCCOCCOCCNC*] | (202) |
| 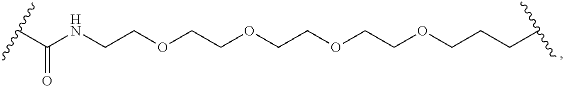 [SMILES: *CCCOCCOCCOCCOCCNC(*)=O] | (203) |
| 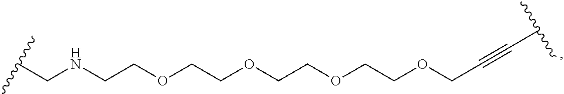 [SMILES: *C#CCOCCOCCOCCOCCNC*] | (204) |
| 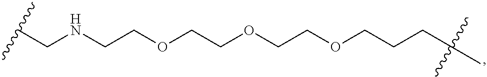 [SMILES: *CCCOCCOCCOCCNC*] | (205) |
| 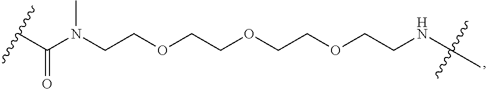 [SMILES: *NCCOCCOCCOCCN(C)C(*)=O] | (206) |
| 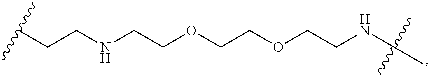 [SMILES: *CCNCCOCCOCCN*] | (207) |
| 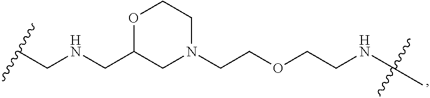 [SMILES: *CNCC1CN(CCOCCN*)CCO1] | (208) |
| 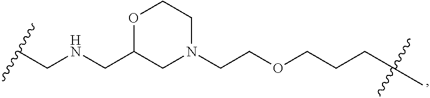 [SMILES: *CCCOCCN1CCOC(CNC*)C1] | (209) |
| 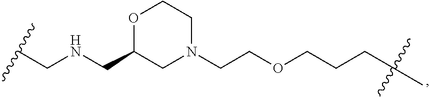 [SMILES: *CCCOCCN1CCO[C@H](CNC*)C1] | (210) |
| 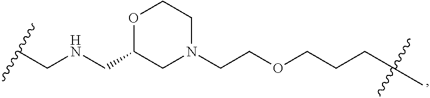 [SMILES: *CCCOCCN1CCO[C@@H](CNC*)C1] | (211) |
| 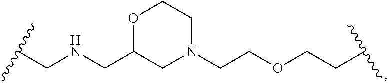 [SMILES: *CCOCCN1CCOC(CNC*)C1] | (212) |
| 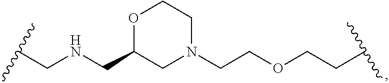 [SMILES: *CCOCCN1CCO[C@H](CNC*)C1] | (213) |
| 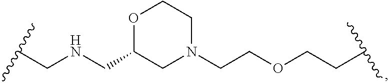 [SMILES: *CCOCCN1CCO[C@@H](CNC*)C1] | (214) |
| 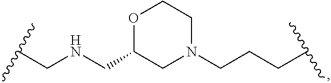 [SMILES: *CCCN1CCO[C@@H](CNC*)C1] | (215) |
| 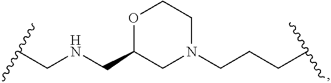 [SMILES: *CCCN1CCO[C@H](CNC*)C1] | (216) |
| 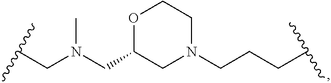 [SMILES: *CCCN1CCO[C@@H](CN(C)C*)C1] | (217) |
| 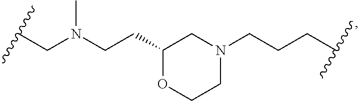 [SMILES: *CCCN1CCO[C@H](CCN(C)C*)C1] | (218) |
| 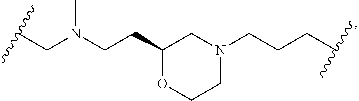 [SMILES: *CCCN1CCO[C@@H](CCN(C)C*)C1] | (219) |
| 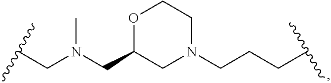 [SMILES: *CCCN1CCO[C@H](CN(C)C*)C1] | (220) |
| 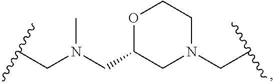 [SMILES: *CN(C)C[C@H]1CN(C*)CCO1] | (221) |
| 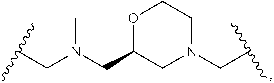 [SMILES: *CN(C)C[C@@H]1CN(C*)CCO1] | (222) |
| 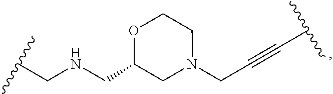 [SMILES: *C#CCN1CCO[C@@H](CNC*)C1] | (223) |
| 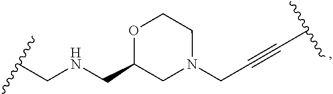 [SMILES: *C#CCN1CCO[C@H](CNC*)C1] | (224) |
| 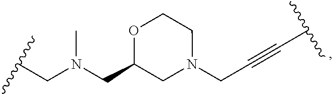 [SMILES: *C#CCN1CCO[C@H](CN(C)C*)C1] | (225) |
| 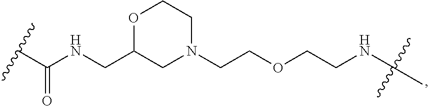 [SMILES: *NCCOCCN1CCOC(CNC(*)=O)C1] | (226) |
| 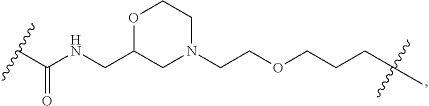 [SMILES: *CCCOCCN1CCOC(CNC(*)=O)C1] | (227) |
| 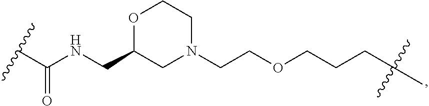 [SMILES: *CCCOCCN1CCO[C@H](CNC(*)=O)C1] | (228) |
| 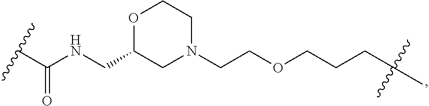 [SMILES: *CCCOCCN1CCO[C@@H](CNC(*)=O)C1] | (229) |
| 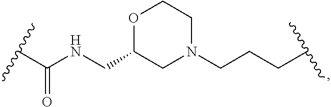 [SMILES: *CCCN1CCO[C@@H](CNC(*)=O)C1] | (230) |
| 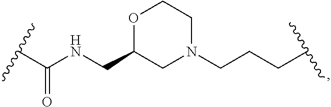 [SMILES: *CCCN1CCO[C@H](CNC(*)=O)C1] | (231) |
| 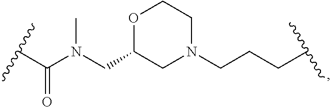 [SMILES: *CCCN1CCO[C@@H](CN(C)C(*)=O)C1] | (232) |
| 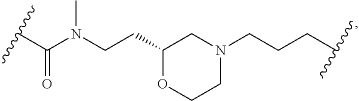 [SMILES: *CCCN1CCO[C@H](CCN(C)C(*)=O)C1] | (233) |
| 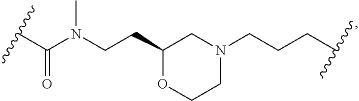 [SMILES: *CCCN1CCO[C@@H](CCN(C)C(*)=O)C1] | (234) |
| 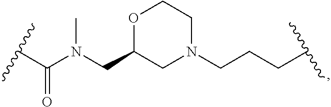 [SMILES: *CCCN1CCO[C@H](CN(C)C(*)=O)C1] | (235) |
| 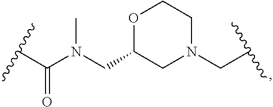 [SMILES: *CN1CCO[C@@H](CN(C)C(*)=O)C1] | (236) |
| 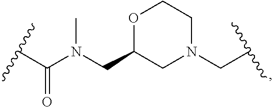 [SMILES: *CN1CCO[C@H](CN(C)C(*)=O)C1] | (237) |
| 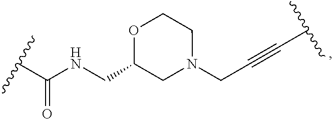 [SMILES: *C#CCN1CCO[C@@H](CNC(*)=O)C1] | (238) |
| 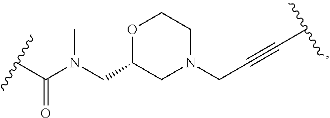 [SMILES: *C#CCN1CCO[C@@H](CN(C)C(*)=O)C1] | (239) |
| 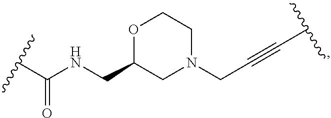 [SMILES: *C#CCN1CCO[C@H](CNC(*)=O)C1] | (240) |
| 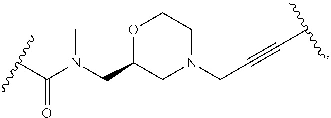 [SMILES: *C#CCN1CCO[C@H](CN(C)C(*)=O)C1] | (241) |
| 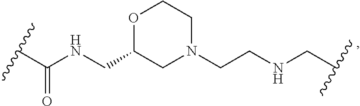 [SMILES: *CNCCN1CCO[C@@H](CNC(*)=O)C1 ; ","] | (242) |
| 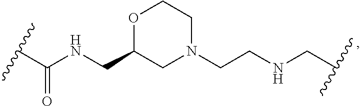 [SMILES: *CNCCN1CCO[C@H](CNC(*)=O)C1 ; ","] | (243) |
| 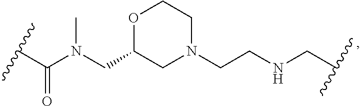 [SMILES: *CNCCN1CCO[C@@H](CN(C)C(*)=O)C1 ; ","] | (244) |
| 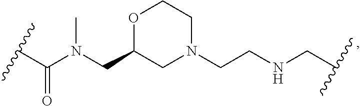 [SMILES: *CNCCN1CCO[C@H](CN(C)C(*)=O)C1 ; ","] | (245) |
| 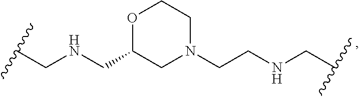 [SMILES: *CNCCN1CCO[C@@H](CNC*)C1 ; ","] | (246) |
| 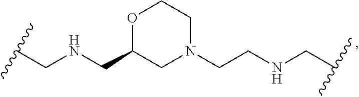 [SMILES: *CNCCN1CCO[C@H](CNC*)C1 ; ","] | (247) |
| 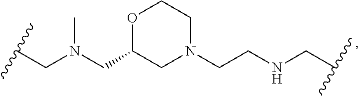 [SMILES: *CNCCN1CCO[C@@H](CN(C)C*)C1 ; ","] | (248) |
| 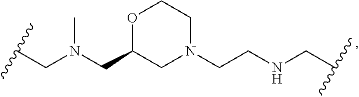 [SMILES: *CNCCN1CCO[C@H](CN(C)C*)C1 ; ","] | (249) |
| 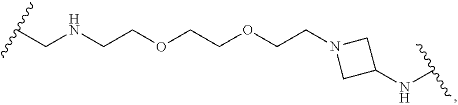 [SMILES: *CNCCOCCOCCN1CC(N*)C1] | (250) |
| 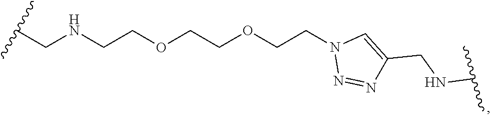 [SMILES: *CNCCOCCOCCn1cc(CN*)nn1] | (251) |
| 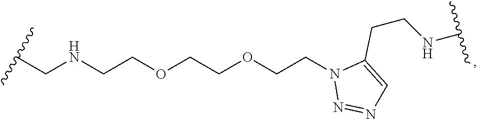 [SMILES: *CNCCOCCOCCn1nncc1CCN*] | (253) |
| 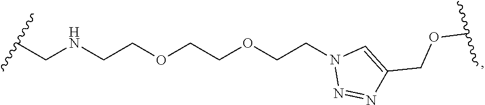 [SMILES: *CNCCOCCOCCn1cc(CO*)nn1] | (254) |
| 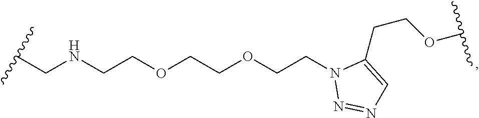 [SMILES: *CNCCOCCOCCn1nncc1CCO*] | (255) |
| 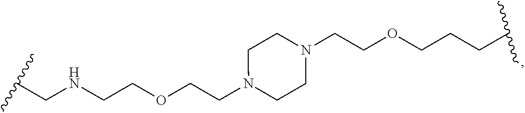 [SMILES: *CCCOCCN1CCN(CCOCCNC*)CC1] | (256) |
| 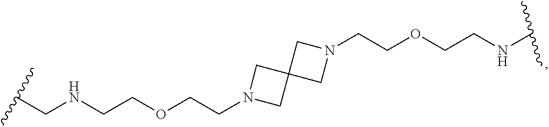 [SMILES: *CNCCOCCN1CC2(CN(CCOCCN*)C2)C1] | (257) |
| 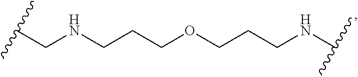 [SMILES: *CNCCCOCCCN*] | (258) |
| 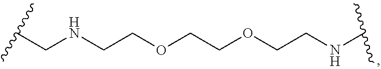 [SMILES: *CNCCOCCOCCN*] | (259) |
| 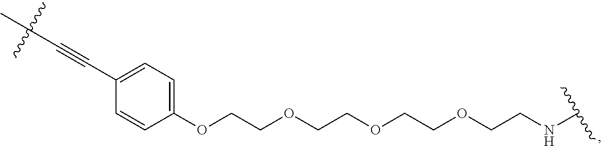 [SMILES: *C#Cc1ccc(OCCOCCOCCOCCN*)cc1] | (260) |
| 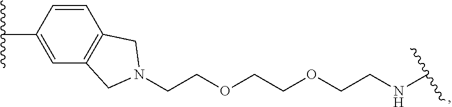 [SMILES: *NCCOCCOCCN1Cc2ccc(*)cc2C1] | (261) |
| 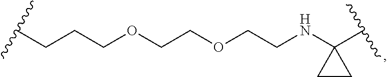 [SMILES: *CCCOCCOCCNC1(*)CC1] | (262) |
| 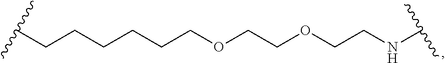 [SMILES: *CCCCCCOCCOCCN*] | (263) |
| 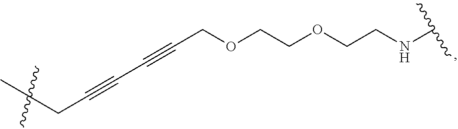 [SMILES: *CC#CC#CCOCCOCCN*] | (264) |
| 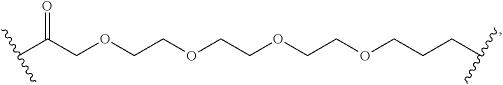 [SMILES: *CCCOCCOCCOCCOCC(*)=O] | (265) |
| 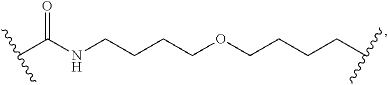 [SMILES: *CCCCOCCCCNC(*)=O] | (266) |
| 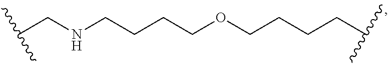 [SMILES: *CCCCOCCCCNC*] | (267) |
| 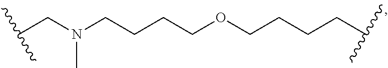 [SMILES: *CCCCOCCCCN(C)C*] | (268) |
| 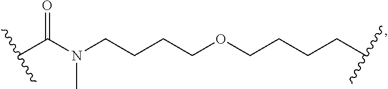 [SMILES: *CCCCOCCCCN(C)C(*)=O] | (269) |
| 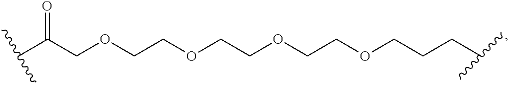 [SMILES: *CCCOCCOCCOCCOCC(*)=O] | (270) |
| 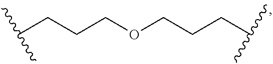 [SMILES: *CCCOCCC*] | (271) |
| 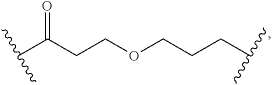 [SMILES: *CCCOCCC(*)=O] | (272) |
| 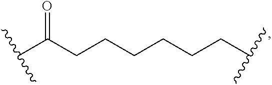 [SMILES: *CCCCCCC(*)=O] | (273) |
| 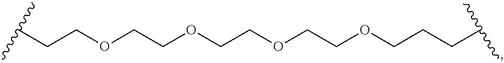 [SMILES: *CCCOCCOCCOCCOCC*] | (274) |
| 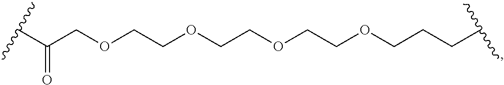 [SMILES: *CCCOCCOCCOCCOCC(*)=O] | (275) |
| 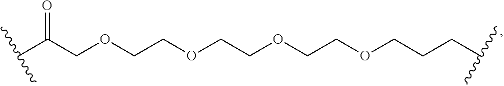 [SMILES: *CCCOCCOCCOCCOCC(*)=O] | (276) |
| 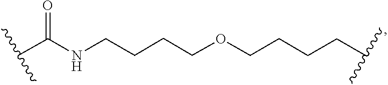 [SMILES: *CCCCOCCCCNC(*)=O] | (277) |
| 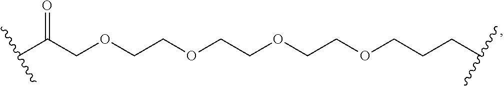 [SMILES: *CCCOCCOCCOCCOCC(*)=O] | (278) |
| 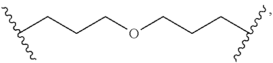 [SMILES: *CCCOCCC*] | (279) |
| 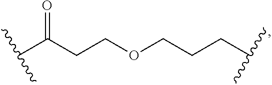 [SMILES: *CCCOCCC(*)=O] | (280) |
| 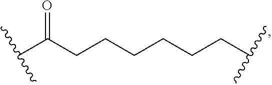 [SMILES: *CCCCCCC(*)=O] | (281) |
| 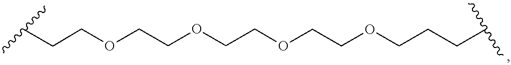 [SMILES: *CCCOCCOCCOCCOCC*] | (282) |
| 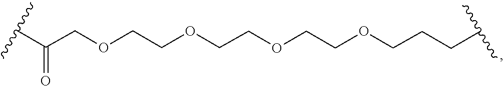 [SMILES: *CCCOCCOCCOCCOCC(*)=O] | (283) |
| 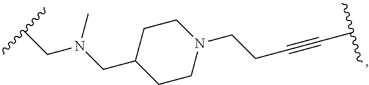 [SMILES: *C#CCCN1CCC(CN(C)C*)CC1] | (284) |
| 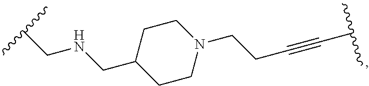 [SMILES: *C#CCCN1CCC(CNC*)CC1] | (285) |
| 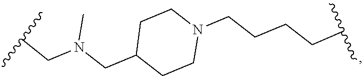 [SMILES: *CCCCN1CCC(CN(C)C*)CC1] | (286) |
| 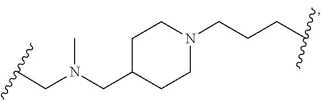 [SMILES: *CCCN1CCC(CN(C)C*)CC1] | (287) |
| 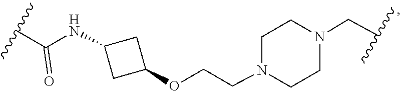 [SMILES: *CN1CCN(CCO[C@H]2C[C@H](NC(*)=O)C2)CC1] | (288) |
| 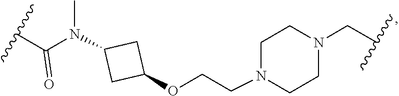 [SMILES: *CN1CCN(CCO[C@H]2C[C@H](N(C)C(*)=O)C2)CC1] | (289) |
| 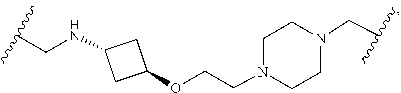 [SMILES: *CN[C@H]1C[C@H](OCCN2CCN(C*)CC2)C1] | (290) |
| 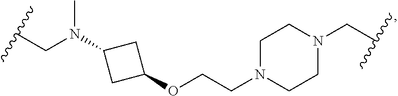 [SMILES: *CN1CCN(CCO[C@H]2C[C@H](N(C)C*)C2)CC1] | (291) |
| 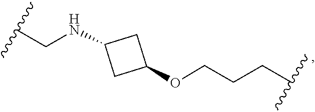 [SMILES: *CCCO[C@H]1C[C@H](NC*)C1] | (292) |
| 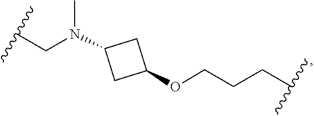 [SMILES: *CCCO[C@H]1C[C@H](N(C)C*)C1] | (293) |
| 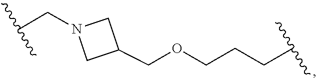 [SMILES: *CCCOCC1CN(C*)C1] | (294) |
| 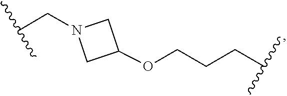 [SMILES: *CCCOC1CN(C*)C1] | (295) |
| 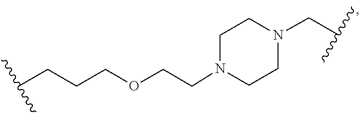 [SMILES: *CCCOCCN1CCN(C*)CC1] | (296) |
| 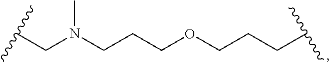 [SMILES: *CCCOCCCN(C)C*] | (297) |
| 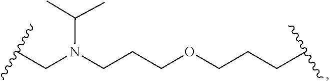 [SMILES: *CCCOCCCN(C*)C(C)C] | (298) |
| 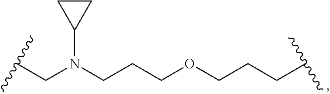 [SMILES: *CCCOCCCN(C*)C1CC1] | (299) |
| 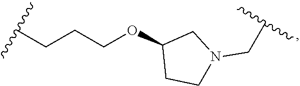 [SMILES: *CCCO[C@@H]1CCN(C*)C1] | (300) |
| 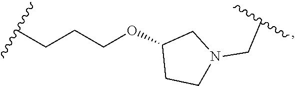 [SMILES: *CCCO[C@H]1CCN(C*)C1] | (301) |
| 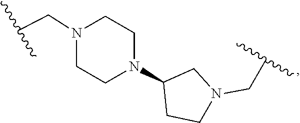 [SMILES: *CN1CCN([C@@H]2CCN(C*)C2)CC1] | (302) |
| 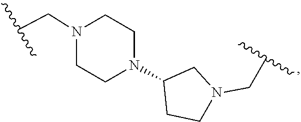 [SMILES: *CN1CCN([C@H]2CCN(C*)C2)CC1] | (303) |
| 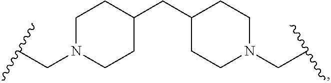 [SMILES: *CN1CCC(CC2CCN(C*)CC2)CC1] | (304) |
| 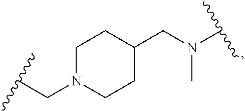 [SMILES: *CN1CCC(CN(*)C)CC1] | (305) |
| 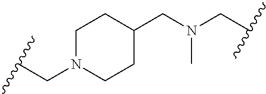 [SMILES: *CN(C)CC1CCN(C*)CC1] | (306) |
| 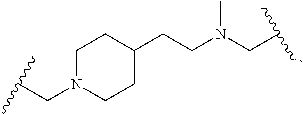 [SMILES: *CN(C)CCC1CCN(C*)CC1] | (307) |
| 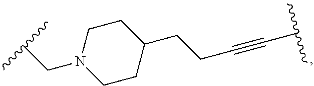 [SMILES: *C#CCCC1CCN(C*)CC1] | (308) |
| 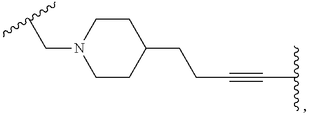 [SMILES: *C#CCCC1CCN(C*)CC1] | (309) |
| 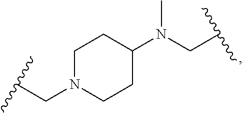 [SMILES: *CN1CCC(N(C)C*)CC1] | (310) |
| 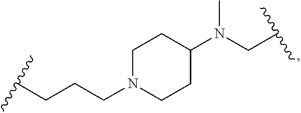 [SMILES: *CCCN1CCC(N(C)C*)CC1] | (311) |
| 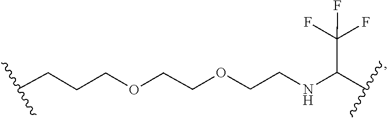 [SMILES: *CCCOCCOCCNC(*)C(F)(F)F] | (312) |
| 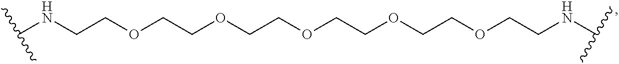 [SMILES: *NCCOCCOCCOCCOCCOCCN*] | (313) |
| 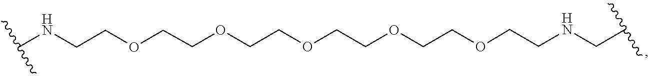 [SMILES: *CNCCOCCOCCOCCOCCOCCN*] | (314) |
| 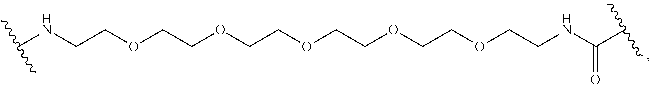 [SMILES: *NCCOCCOCCOCCOCCOCCNC(*)=O] | (315) |
| 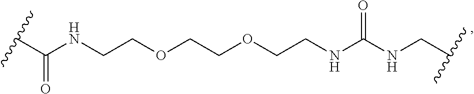 [SMILES: *CNC(=O)NCCOCCOCCNC(*)=O] | (316) |
| 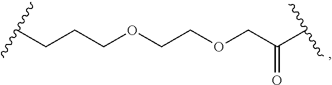 [SMILES: *CCCOCCOCC(*)=O] | (317) |
| 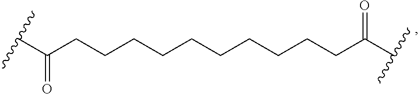 [SMILES: *C(=O)CCCCCCCCCCC(*)=O] | (318) |
| 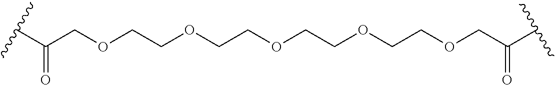 [SMILES: *C(=O)COCCOCCOCCOCCOCC(*)=O] | (319) |
| 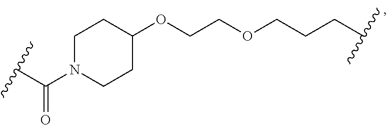 [SMILES: *CCCOCCOC1CCN(C(*)=O)CC1] | (320) |
| 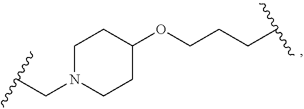 [SMILES: *CCCOC1CCN(C*)CC1] | (321) |
| 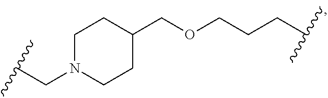 [SMILES: *CCCOCC1CCN(C*)CC1] | (322) |
| 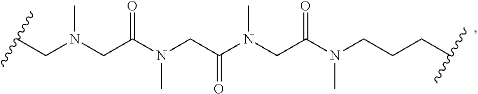 [SMILES: *CCCN(C)C(=O)CN(C)C(=O)CN(C)C(=O)CN(C)C*] | (323) |
| 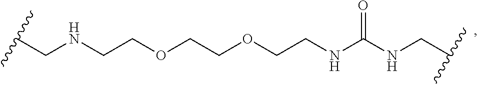 [SMILES: *CNCCOCCOCCNC(=O)NC*] | (324) |
| 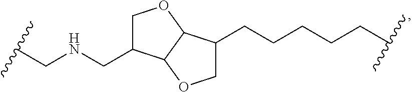 [SMILES: *CCCCCC1COC2C(CNC*)COC12] | (325) |
| 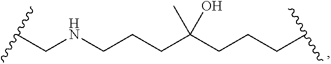 [SMILES: *CCCC(C)(O)CCCNC*] | (326) |
| 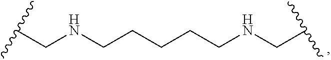 [SMILES: *CNCCCCCNC*] | (327) |
| 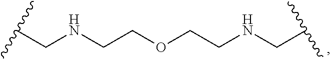 [SMILES: *CNCCOCCNC*] | (328) |
| 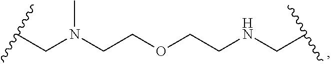 [SMILES: *CNCCOCCN(C)C*] | (329) |
| 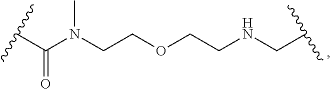 [SMILES: *CNCCOCCN(C)C(*)=O] | (330) |
| 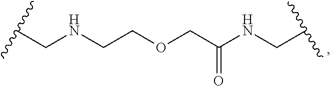 [SMILES: *CNCCOCC(=O)NC*] | (331) |
| 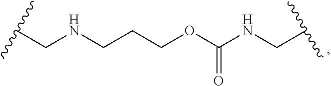 [SMILES: *CNCCCOC(=O)NC*] | (332) |
| 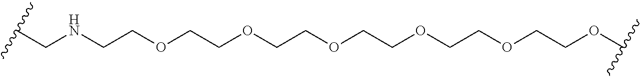 [SMILES: *CNCCOCCOCCOCCOCCOCCO*] | (333) |
| 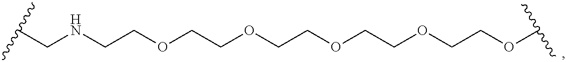 [SMILES: *CNCCOCCOCCOCCOCCO*] | (334) |
| 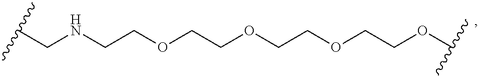 [SMILES: *CNCCOCCOCCOCCO*] | (335) |
| 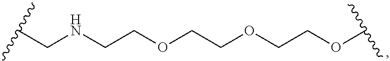 [SMILES: *CNCCOCCOCCO*] | (336) |
| 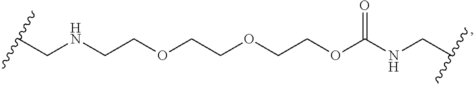 [SMILES: *CNCCOCCOCCOC(=O)NC*] | (337) |
| 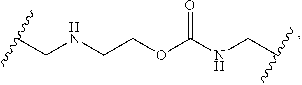 [SMILES: *CNCCOC(=O)NC*] | (338) |
| 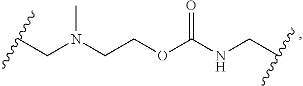 [SMILES: *CNC(=O)OCCN(C)C*] | (339) |
| 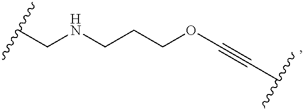 [SMILES: *C#COCCCNC*] | (340) |
| 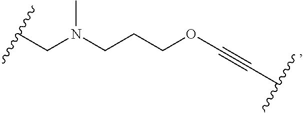 [SMILES: *C#COCCCN(C)C*] | (341) |
| 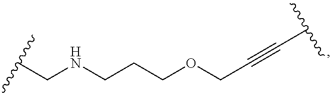 [SMILES: *C#CCOCCCNC*] | (342) |
| 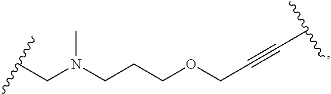 [SMILES: *C#CCOCCCN(C)C*] | (343) |
| 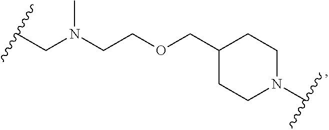 [SMILES: *CN(C)CCOCC1CCN(*)CC1] | (344) |
| 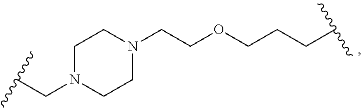 [SMILES: *CCCOCCN1CCN(C*)CC1] | (345) |
| 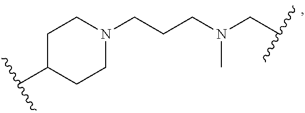 [SMILES: *CN(C)CCCN1CCC(*)CC1] | (346) |
| 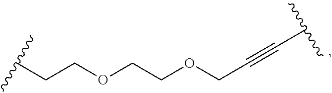 [SMILES: *C#CCOCCOCC*] | (347) |
| 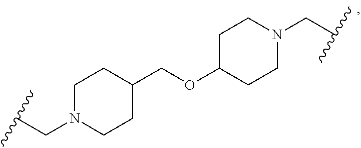 [SMILES: *CN1CCC(COC2CCN(C*)CC2)CC1] | (348) |
| 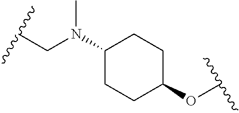 [SMILES: *CN(C)[C@H]1CC[C@H](O*)CC1] | (349) |
| 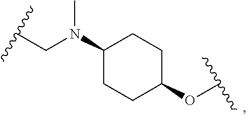 [SMILES: *CN(C)[C@H]1CC[C@@H](O*)CC1] | (350) |
| 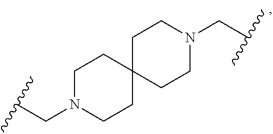 [SMILES: *CN1CCC2(CC1)CCN(C*)CC2] | (351) |
| 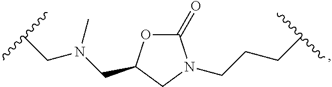 [SMILES: *CCCN1C[C@@H](CN(C)C*)OC1=O] | (352) |
| 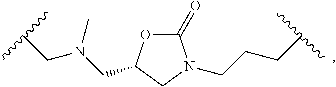 [SMILES: *CCCN1C[C@H](CN(C)C*)OC1=O] | (353) |
| 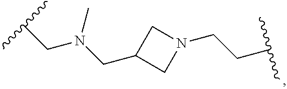 [SMILES: *CCN1CC(CN(C)C*)C1] | (354) |
| 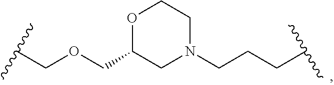 [SMILES: *CCCN1CCO[C@@H](COC*)C1] | (355) |
| 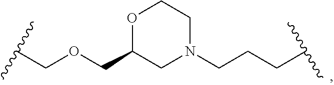 [SMILES: *CCCN1CCO[C@H](COC*)C1] | (356) |
| 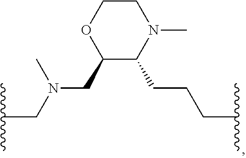 [SMILES: *CCC[C@@H]1[C@@H](CN(C)C*)OCCN1C] | (357) |
| 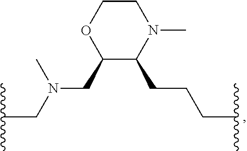 [SMILES: *CCC[C@H]1[C@@H](CN(C)C*)OCCN1C] | (358) |
| 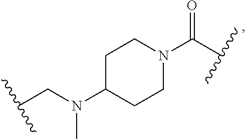 [SMILES: *CN(C)C1CCN(C(*)=O)CC1] | (359) |
| 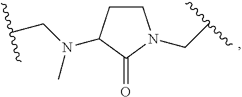 [SMILES: *CN1CCC(N(C)C*)C1=O] | (360) |
| 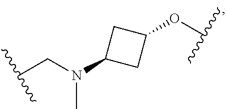 [SMILES: *CN(C)[C@H]1C[C@H](O*)C1] | (361) |
| 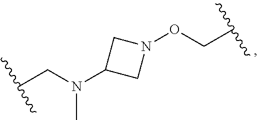 [SMILES: *CON1CC(N(C)C*)C1] | (362) |
| 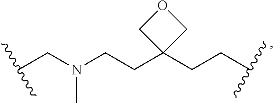 [SMILES: *CCC1(CCN(C)C*)COC1] | (363) |
| 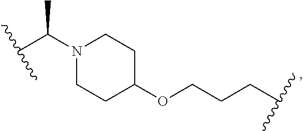 [SMILES: *CCCOC1CCN([C@@H](*)C)CC1] | (364) |
| 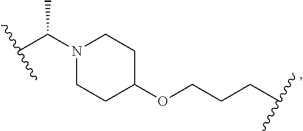 [SMILES: *CCCOC1CCN([C@H](*)C)CC1] | (365) |
| 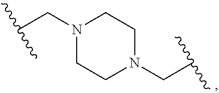 [SMILES: *CN1CCN(C*)CC1] | (366) |
| 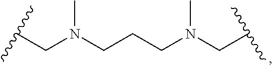 [SMILES: *CN(C)CCCN(C)C*] | (367) |
| 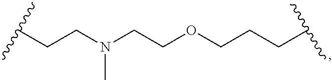 [SMILES: *CCCOCCN(C)CC*] | (368) |
| 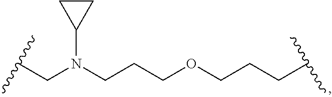 [SMILES: *CCCOCCCN(C*)C1CC1] | (369) |
| 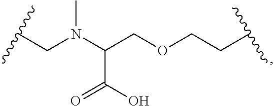 [SMILES: *CCOCC(C(=O)O)N(C)C*] | (370) |
| 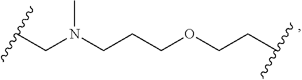 [SMILES: *CCOCCCN(C)C*] | (371) |
| 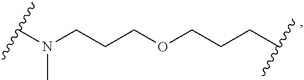 [SMILES: *CCCOCCCN(*)C] | (372) |
| 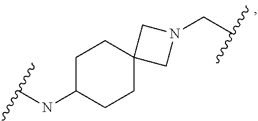 [SMILES: *CN1CC2(CCC(N*)CC2)C1] | (373) |
| 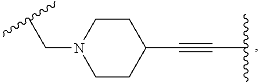 [SMILES: *C#CC1CCN(C*)CC1] | (374) |
| 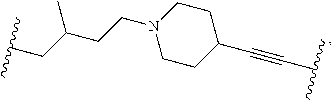 [SMILES: *C#CC1CCN(CCC(C)C*)CC1] | (375) |
| 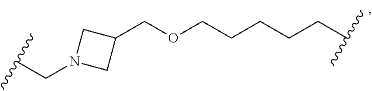 [SMILES: *CCCCCOCC1CN(C*)C1] | (376) |
| 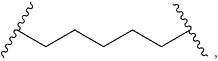 [SMILES: *CCCCC*] | (377) |
| 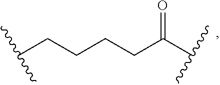 [SMILES: *CCCCC(*)=O] | (378) |
| 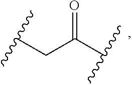 [SMILES: *CC(*)=O] | (379) |
| 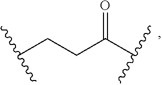 [SMILES: *CCC(*)=O] | (380) |
| 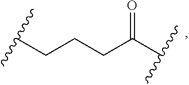 [SMILES: *CCCC(*)=O] | (381) |
| 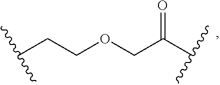 [SMILES: *CCOCC(*)=O] | (382) |
| 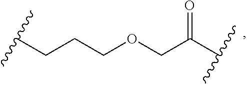 [SMILES: *CCCOCC(*)=O] | (383) |
| 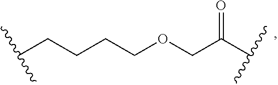 [SMILES: *CCCCOCC(*)=O] | (384) |
| 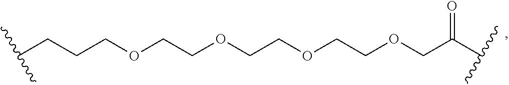 [SMILES: *CCCOCCOCCOCCOCC(*)=O] | (385) |
| 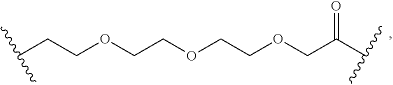 [SMILES: *CCOCCOCCOCC(*)=O] | (386) |
| 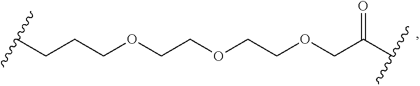 [SMILES: *CCCOCCOCCOCC(*)=O] | (387) |
| 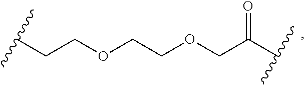 [SMILES: *CCOCCOCC(*)=O] | (388) |
| 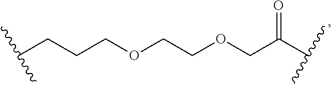 [SMILES: *CCCOCCOCC(*)=O] | (389) |
| 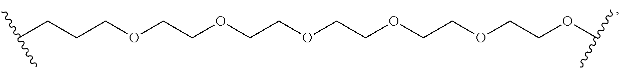 [SMILES: *CCCOCCOCCOCCOCCOCCO*] | (390) |
| 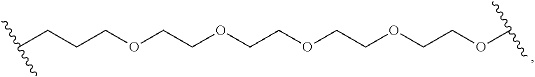 [SMILES: *CCCOCCOCCOCCOCCO*] | (391) |
| 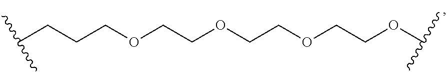 [SMILES: *CCCOCCOCCOCCO*] | (392) |
| 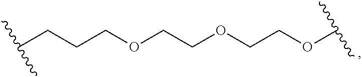 [SMILES: *CCCOCCOCCO*] | (393) |
| 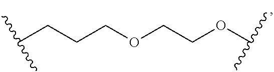 [SMILES: *CCCOCCO*] | (394) |
| 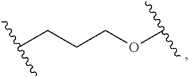 [SMILES: *CCCO*] | (395) |
| 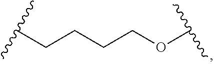 [SMILES: *CCCCO*] | (396) |
| 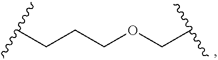 [SMILES: *CCCOC*] | (397) |
| 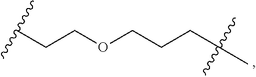 [SMILES: *CCCOCC*] | (398) |
| 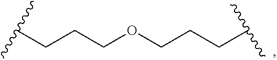 [SMILES: *CCCOCCC*] | (399) |
| 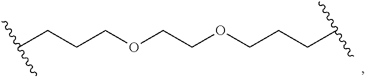 [SMILES: *CCCOCCOCCC*] | (400) |
| 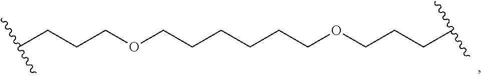 [SMILES: *CCCOCCCCCCOCCC*] | (401) |
| 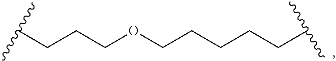 [SMILES: *CCCCCOCCC*] | (402) |
| 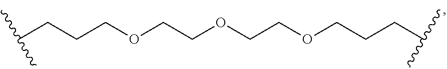 [SMILES: *CCCOCCOCCOCCC*] | (403) |
| 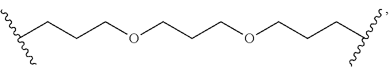 [SMILES: *CCCOCCCOCCC*] | (404) |
| 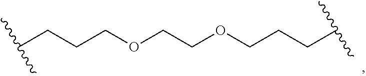 [SMILES: *CCCOCCOCCC*] | (405) |
| 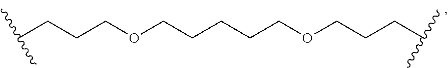 [SMILES: *CCCOCCCCCOCCC*] | (406) |
| 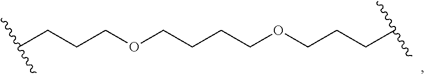 [SMILES: *CCCOCCCCOCCC*] | (407) |
| 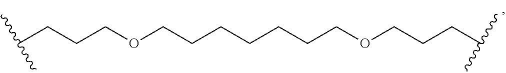 [SMILES: *CCCOCCCCCCCOCCC*] | (408) |
| 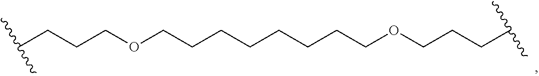 [SMILES: *CCCOCCCCCCCCOCCC*] | (409) |
| 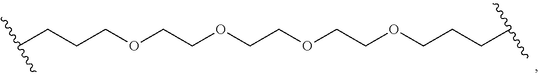 [SMILES: *CCCOCCOCCOCCOCCC*] | (410) |
| 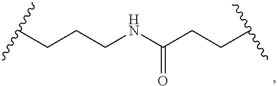 [SMILES: *CCCNC(=O)CC*] | (411) |
| 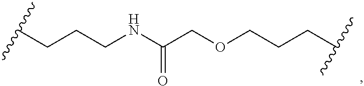 [SMILES: *CCCNC(=O)COCCC*] | (412) |
| 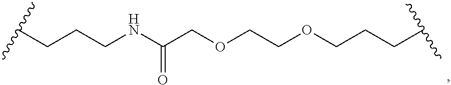 [SMILES: *CCCNC(=O)COCCOCCC*] | (413) |
| 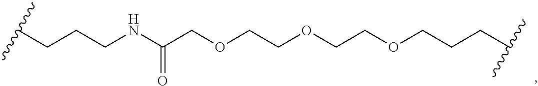 [SMILES: *CCCNC(=O)COCCOCCOCCC*] | (414) |
| 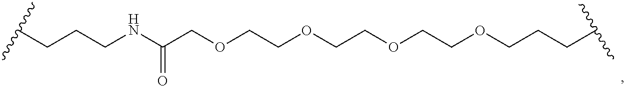 [SMILES: *CCCNC(=O)COCCOCCOCCOCCC*] | (415) |
| 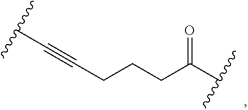 [SMILES: *C#CCCCC(*)=O] | (416) |
| 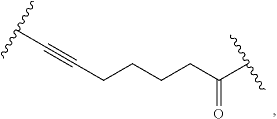 [SMILES: *C#CCCCCC(*)=O] | (417) |
| 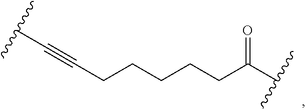 [SMILES: *C#CCCCCCC(*)=O] | (418) |
| 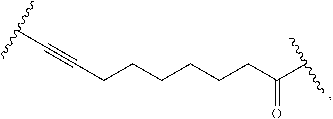 [SMILES: *C#CCCCCCCC(*)=O] | (419) |
| 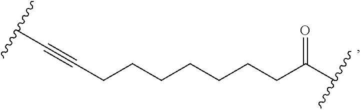 [SMILES: *C#CCCCCCCCC(*)=O] | (420) |
| 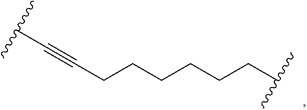 [SMILES: *C#CCCCCCC*] | (421) |
| 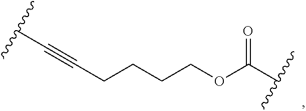 [SMILES: *C#CCCCCOC(*)=O] | (422) |
| 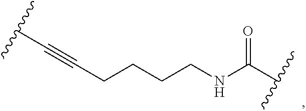 [SMILES: *C#CCCCCNC(*)=O] | (423) |
| 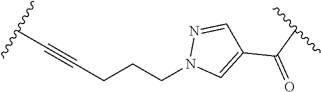 [SMILES: *C#CCCCn1cc(C(*)=O)cn1] | (424) |
| 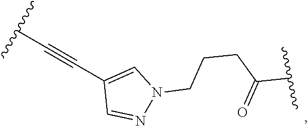 [SMILES: *C#Cc1cnn(CCCC(*)=O)c1] | (425) |
| 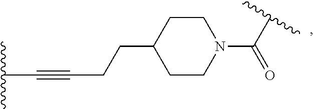 [SMILES: *C#CCCC1CCN(C(*)=O)CC1] | (426) |
| 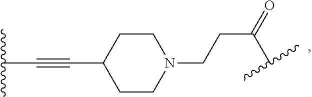 [SMILES: *C#CC1CCN(CCC(*)=O)CC1] | (427) |
| 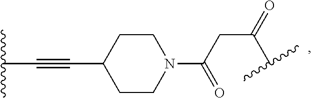 [SMILES: *C#CC1CCN(C(=O)CC(*)=O)CC1] | (428) |
| 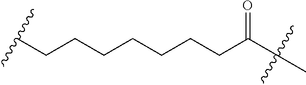 [SMILES: *CCCCCCCC(*)=O] | (429) |
| 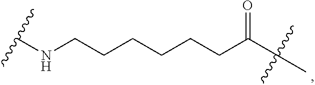 [SMILES: *NCCCCCCC(*)=O] | (430) |
| 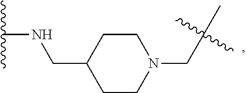 [SMILES: *CN1CCC(CN*)CC1] | (431) |
| 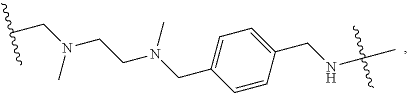 [SMILES: *CN(C)CCN(C)Cc1ccc(CN*)cc1] | (432) |
| 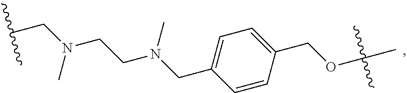 [SMILES: *CN(C)CCN(C)Cc1ccc(CO*)cc1] | (433) |
| 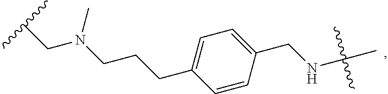 [SMILES: *CN(C)CCCc1ccc(CN*)cc1] | (434) |
| 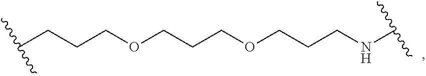 [SMILES: *CCCOCCCOCCCN*] | (435) |
| 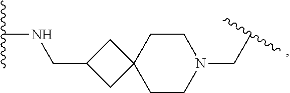 [SMILES: *CN1CCC2(CC1)CC(CN*)C2] | (436) |
| 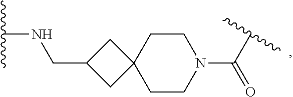 [SMILES: *NCC1CC2(CCN(C(*)=O)CC2)C1] | (437) |
| 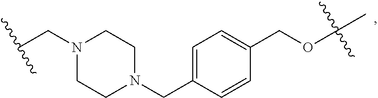 [SMILES: *CN1CCN(Cc2ccc(CO*)cc2)CC1] | (438) |
| 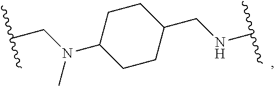 [SMILES: *CN(C)C1CCC(CN*)CC1] | (438) |
| 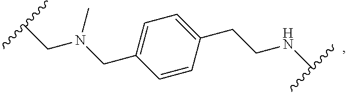 [SMILES: *CN(C)Cc1ccc(CCN*)cc1] | (439) |
| 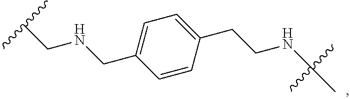 [SMILES: *CNCc1ccc(CCN*)cc1] | (440) |
| 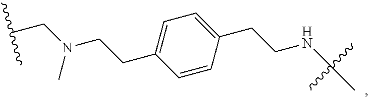 [SMILES: *CN(C)CCc1ccc(CCN*)cc1] | (441) |
| 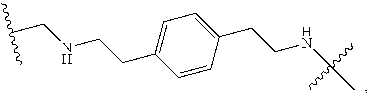 [SMILES: *CNCCc1ccc(CCN*)cc1] | (442) |
| 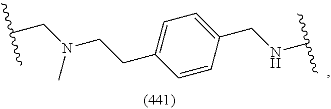 [SMILES: *CN(C)CCc1ccc(CN*)cc1] | (443) |
| 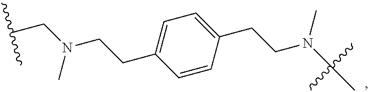 [SMILES: *CN(C)CCc1ccc(CCN(*)C)cc1] | (444) |
| 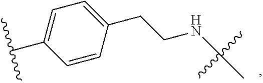 [SMILES: *NCCc1ccc(*)cc1] | (445) |
| 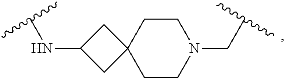 [SMILES: *CN1CCC2(CC1)CC(N*)C2] | (446) |
| 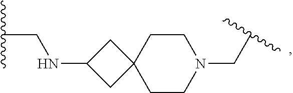 [SMILES: *CNC1CC2(CCN(C*)CC2)C1] | (447) |
| 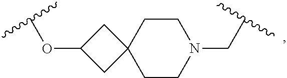 [SMILES: *CN1CCC2(CC1)CC(O*)C2] | (448) |
| 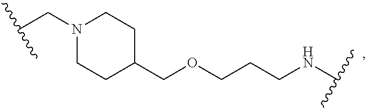 [SMILES: *CN1CCC(COCCCN*)CC1] | (449) |
| 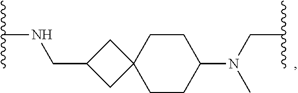 [SMILES: *CN(C)C1CCC2(CC1)CC(CN*)C2] | (450) |
| 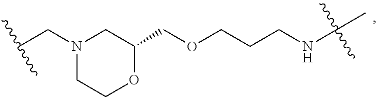 [SMILES: *CN1CCO[C@@H](COCCCN*)C1] | (451) |
| 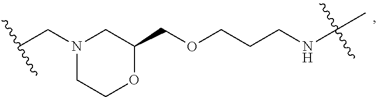 [SMILES: *CN1CCO[C@H](COCCCN*)C1] | (452) |
| 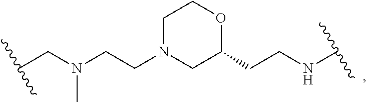 [SMILES: *CN(C)CCN1CCO[C@H](CCN*)C1] | (453) |
| 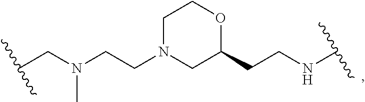 [SMILES: *CN(C)CCN1CCO[C@@H](CCN*)C1] | (454) |
| 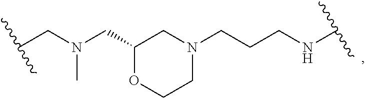 [SMILES: *CN(C)C[C@@H]1CN(CCCN*)CCO1] | (455) |
| 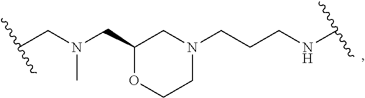 [SMILES: *CN(C)C[C@H]1CN(CCCN*)CCO1] | (456) |
| 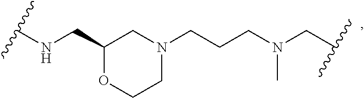 [SMILES: *CN(C)CCCN1CCO[C@@H](CN*)C1] | (457) |
| 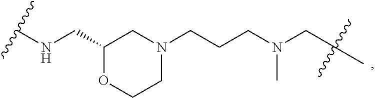 [SMILES: *CN(C)CCCN1CCO[C@H](CN*)C1] | (458) |
| 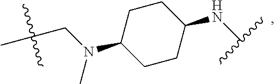 [SMILES: *CN(C)[C@H]1CC[C@@H](N*)CC1] | (459) |
| 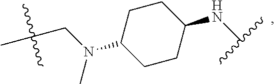 | (460) |
| 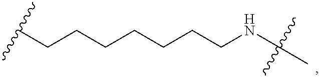 [SMILES: *CCCCCCCN*] | (461) |
| 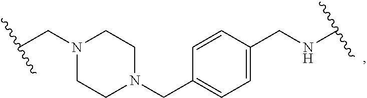 [SMILES: *CN1CCN(Cc2ccc(CN*)cc2)CC1] | (462) |
| 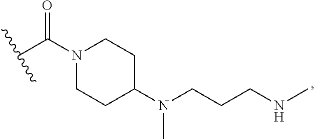 [SMILES: *C(=O)N1CCC(N(C)CCCNC)CC1] | (463) |
| 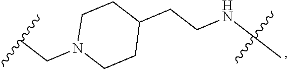 [SMILES: *CN1CCC(CCN*)CC1] | (464) |
| 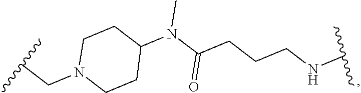 [SMILES: *CN1CCC(N(C)C(=O)CCCN*)CC1] | (465) |
| 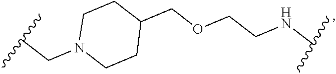 [SMILES: *CN1CCC(COCCN*)CC1] | (466) |
| 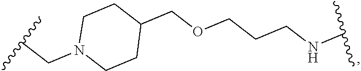 [SMILES: *CN1CCC(COCCCN*)CC1] | (467) |
| 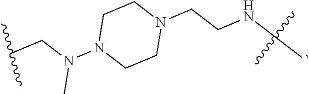 [SMILES: *CN(C)N1CCN(CCN*)CC1] | (468) |
| 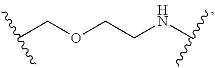 [SMILES: *COCCN*] | (469) |
| 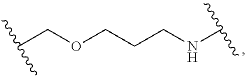 [SMILES: *COCCCN*] | (470) |
| 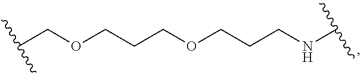 [SMILES: *COCCCOCCCN*] | (471) |
| 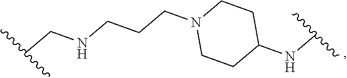 [SMILES: *CNCCCN1CCC(N*)CC1] | (472) |
| 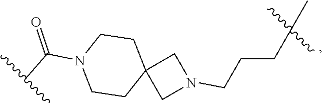 [SMILES: *CCCN1CC2(CCN(C(*)=O)CC2)C1] | (473) |
| 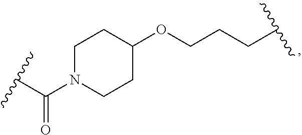 [SMILES: *CCCOC1CCN(C(*)=O)CC1] | (474) |
| 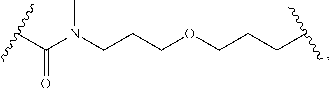 [SMILES: *CCCOCCCN(C)C(*)=O] | (475) |
| 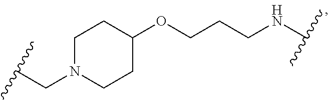 [SMILES: *CN1CCC(OCCCN*)CC1] | (475) |
| 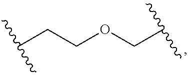 [SMILES: *CCOC*] | (476) |
| 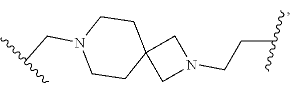 [SMILES: *CCN1CC2(CCN(C*)CC2)C1] | (477) |
| 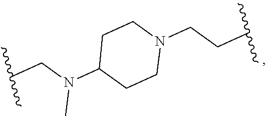 [SMILES: *CCN1CCC(N(C)C*)CC1] | (478) |
| 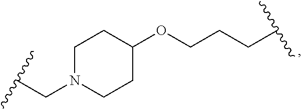 [SMILES: *CCCOC1CCN(C*)CC1] | (479) |
| 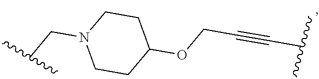 [SMILES: *C#CCOC1CCN(C*)CC1] | (480) |
| 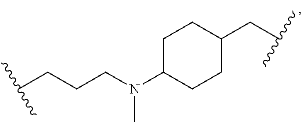 [SMILES: *CCCN(C)C1CCC(C*)CC1] | (481) |
| 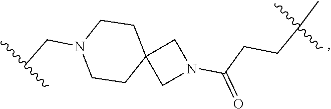 [SMILES: *CCC(=O)N1CC2(CCN(C*)CC2)C1] | (482) |
| 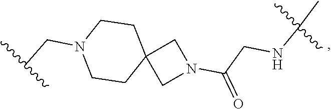 [SMILES: *CN1CCC2(CC1)CN(C(=O)CN*)C2] | (483) |
| 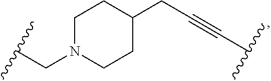 [SMILES: *C#CCC1CCN(C*)CC1] | (484) |
| 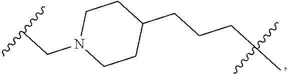 [SMILES: *CCCC1CCN(C*)CC1] | (485) |
| 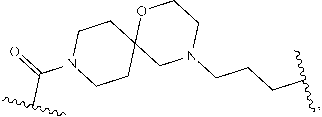 [SMILES: *CCCN1CCOC2(CCN(C(*)=O)CC2)C1] | (486) |
| 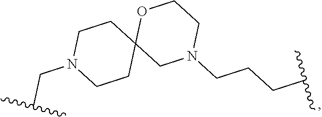 [SMILES: *CCCN1CCOC2(CCN(C*)CC2)C1] | (487) |
| 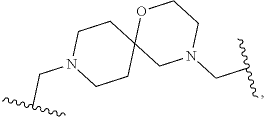 [SMILES: *CN1CCC2(CC1)CN(C*)CCO2] | (488) |
| 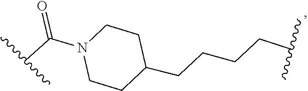 [SMILES: *CCCCC1CCN(C(*)=O)CC1] | (489) |
| 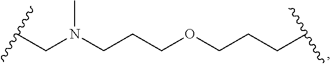 [SMILES: *CCCOCCCN(C)C*] | (490) |
| 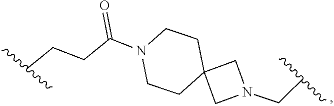 [SMILES: *CCC(=O)N1CCC2(CC1)CN(C*)C2] | (491) |
| 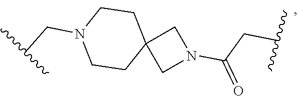 [SMILES: *CC(=O)N1CC2(CCN(C*)CC2)C1] | (492) |
| 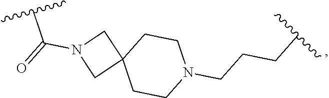 [SMILES: *CCCN1CCC2(CC1)CN(C(*)=O)C2] | (493) |
| 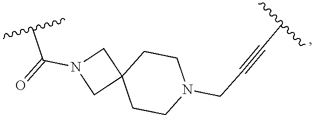 [SMILES: *C#CCN1CCC2(CC1)CN(C(*)=O)C2] | (494) |
| 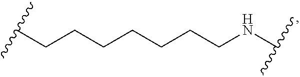 [SMILES: *CCCCCCCN*] | (495) |
| 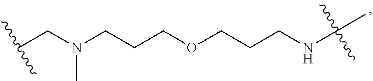 [SMILES: *CN(C)CCCOCCCN*] | (496) |
| 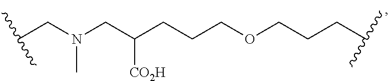 [SMILES: *CCCOCCCC(CN(C)C*)C(=O)O] | (497) |
| 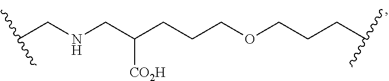 [SMILES: *CCCOCCCC(CNC*)C(=O)O] | (498) |
| 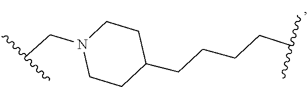 [SMILES: *CCCCC1CCN(C*)CC1] | (499) |
| 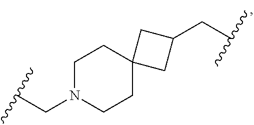 [SMILES: *CC1CC2(CCN(C*)CC2)C1] | (500) |
| 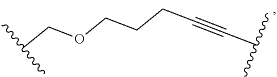 [SMILES: *C#CCCCOC*] | (501) |
| 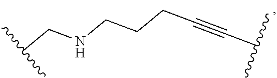 [SMILES: *C#CCCCNC*] | (502) |
| 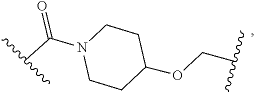 [SMILES: *COC1CCN(C(*)=O)CC1] | (503) |
| 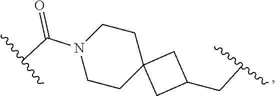 [SMILES: *CC1CC2(CCN(C(*)=O)CC2)C1] | (504) |
| 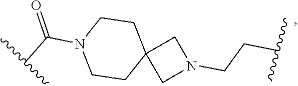 [SMILES: *CCN1CC2(CCN(C(*)=O)CC2)C1] | (505) |
| 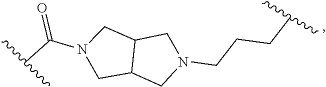 [SMILES: *CCCN1CC2CN(C(*)=O)CC2C1] | (506) |
| 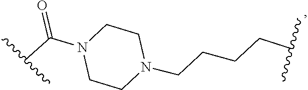 [SMILES: *CCCCN1CCN(C(*)=O)CC1] | (507) |
| 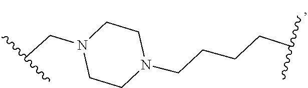 [SMILES: *CCCCN1CCN(C*)CC1] | (508) |
| 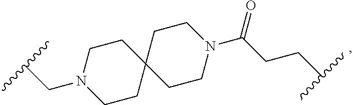 [SMILES: *CCC(=O)N1CCC2(CCN(C*)CC2)CC1] | (509) |
| 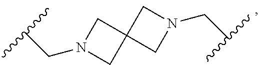 [SMILES: *CN1CC2(C1)CN(C*)C2] | (510) |
| 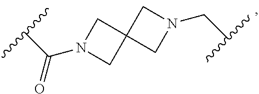 [SMILES: *CN1CC2(C1)CN(C(*)=O)C2] | (511) |
| 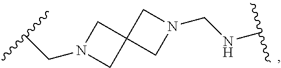 [SMILES: *CN1CC2(C1)CN(CN*)C2] | (512) |
| 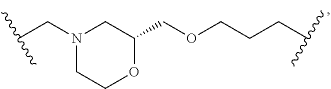 [SMILES: *CCCOC[C@H]1CN(C*)CCO1] | (513) |
| 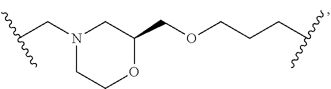 [SMILES: *CCCOC[C@@H]1CN(C*)CCO1] | (514) |
| 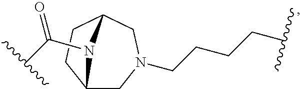 [SMILES: *CCCCN1C[C@H]2CC[C@@H](C1)N2C(*)=O] | (515) |
| 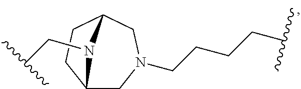 [SMILES: *CCCCN1C[C@H]2CC[C@@H](C1)N2C*] | (516) |
| 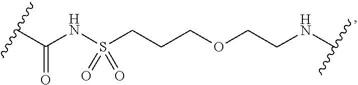 [SMILES: *NCCOCCCS(=O)(=O)NC(*)=O] | (517) |
| 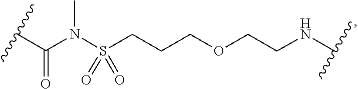 [SMILES: *NCCOCCCS(=O)(=O)N(C)C(*)=O] | (518) |
| 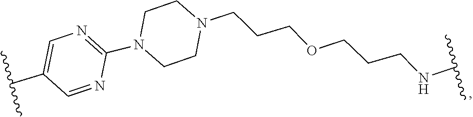 [SMILES: *NCCCOCCCN1CCN(c2ncc(*)cn2)CC1] | (519) |
| 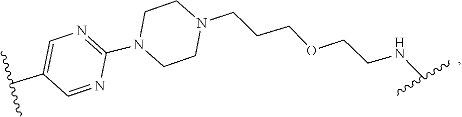 [SMILES: *NCCOCCCN1CCN(c2ncc(*)cn2)CC1] | (520) |
| 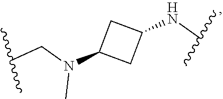 [SMILES: *CN(C)[C@H]1C[C@H](N*)C1] | (521) |
| 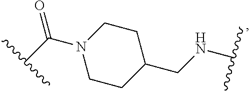 [SMILES: *NCC1CCN(C(*)=O)CC1] | (522) |
| 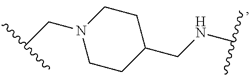 [SMILES: *CN1CCC(CN*)CC1] | (523) |
| 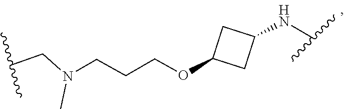 [SMILES: *CN(C)CCCO[C@H]1C[C@H](N*)C1] | (524) |
| 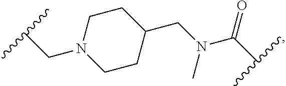 [SMILES: *CN1CCC(CN(C)C(*)=O)CC1] | (525) |
| 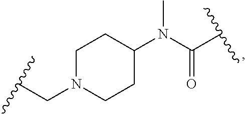 [SMILES: *CN1CCC(N(C)C(*)=O)CC1] | (526) |
| 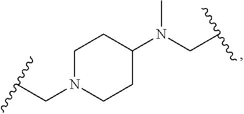 [SMILES: *CN1CCC(N(C)C*)CC1] | (527) |
| 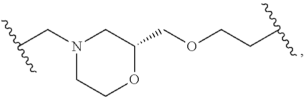 [SMILES: *CCOC[C@H]1CN(C*)CCO1] | (528) |
| 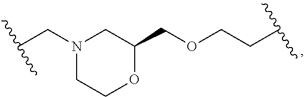 [SMILES: *CCOC[C@@H]1CN(C*)CCO1] | (529) |
| 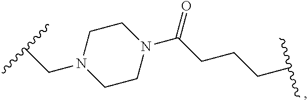 [SMILES: *CCCC(=O)N1CCN(C*)CC1] | (530) |
| 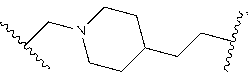 [SMILES: *CCC1CCN(C*)CC1] | (531) |
| 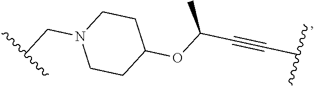 [SMILES: *C#C[C@H](C)OC1CCN(C*)CC1] | (532) |
| 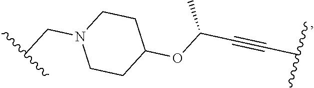 [SMILES: *C#C[C@@H](C)OC1CCN(C*)CC1] | (533) |
| 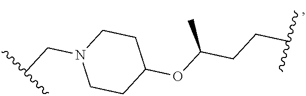 [SMILES: *CC[C@H](C)OC1CCN(C*)CC1] | (534) |
| 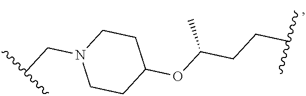 [SMILES: *CC[C@@H](C)OC1CCN(C*)CC1] | (535) |
| 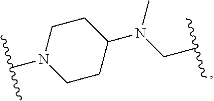 [SMILES: *CN(C)C1CCN(*)CC1] | (536) |
| 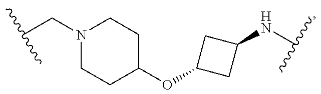 [SMILES: *CN1CCC(O[C@H]2C[C@H](N*)C2)CC1] | (537) |
| 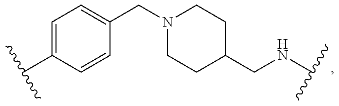 [SMILES: *NCC1CCN(Cc2ccc(*)cc2)CC1] | (538) |
| 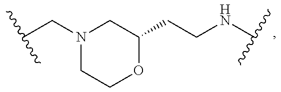 [SMILES: *CN1CCO[C@@H](CCN*)C1] | (539) |
| 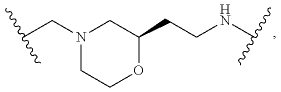 [SMILES: *CN1CCO[C@H](CCN*)C1] | (540) |
| 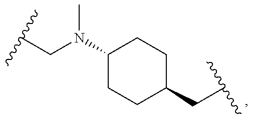 [SMILES: *CN(C)[C@H]1CC[C@H](C*)CC1] | (541) |
| 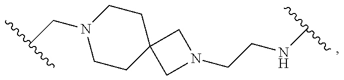 [SMILES: *CN1CCC2(CC1)CN(CCN*)C2] | (542) |
| 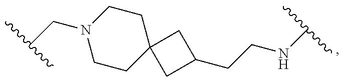 [SMILES: *CN1CCC2(CC1)CC(CCN*)C2] | (543) |
| 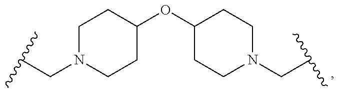 [SMILES: *CN1CCC(OC2CCN(C*)CC2)CC1] | (544) |
| 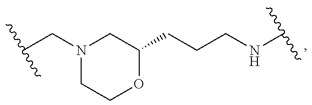 [SMILES: *CN1CCO[C@@H](CCCN*)C1] | (545) |
| 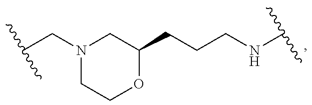 [SMILES: *CN1CCO[C@H](CCCN*)C1] | (546) |
| 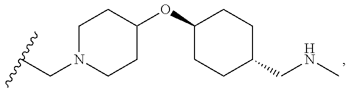 [SMILES: *CN1CCC(O[C@H]2CC[C@H](CNC)CC2)CC1] | (547) |
| 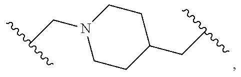 [SMILES: *CC1CCN(C*)CC1] | (548) |
| 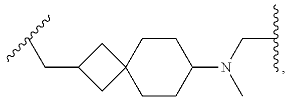 [SMILES: *CC1CC2(CCC(N(C)C*)CC2)C1] | (549) |
| 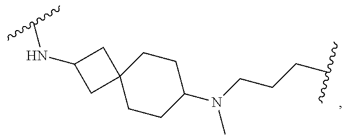 [SMILES: *CCCN(C)C1CCC2(CC1)CC(N*)C2] | (550) |
| 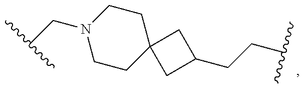 [SMILES: *CCC1CC2(CCN(C*)CC2)C1] | (551) |
| 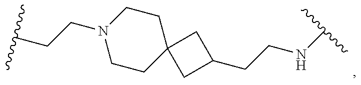 [SMILES: *CCN1CCC2(CC1)CC(CCN*)C2] | (552) |
| 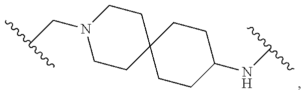 [SMILES: *CN1CCC2(CCC(N*)CC2)CC1] | (553) |
| 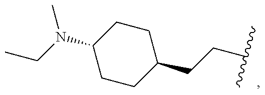 [SMILES: *CC[C@H]1CC[C@H](N(C)CC)CC1] | (554) |
| 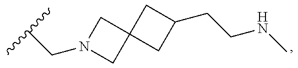 [SMILES: *CN1CC2(CC(CCNC)C2)C1] | (555) |
| 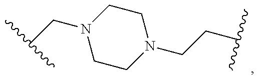 [SMILES: *CCN1CCN(C*)CC1] | (556) |
| 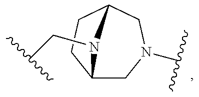 [SMILES: *CN1[C@@H]2CC[C@H]1CN(*)C2] | (557) |
| 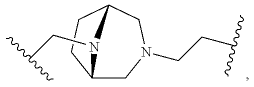 [SMILES: *CCN1C[C@H]2CC[C@@H](C1)N2C*] | (558) |
| 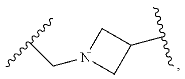 [SMILES: *CN1CC(*)C1] | (559) |
| 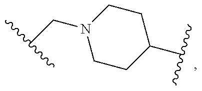 [SMILES: *CN1CCC(*)CC1] | (560) |
| 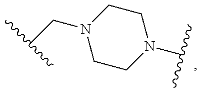 [SMILES: *CN1CCN(*)CC1] | (561) |
| 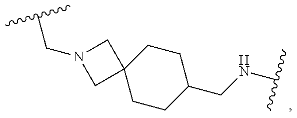 [SMILES: *CN1CC2(CCC(CN*)CC2)C1] | (562) |
| 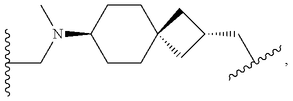 | (563) |
| 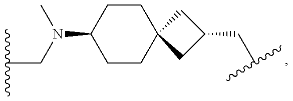 | (564) |
| 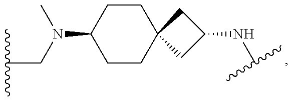 | (565) |
| 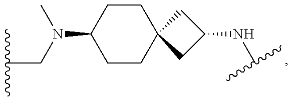 | (566) |
| 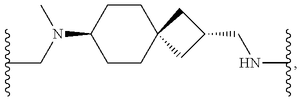 | (567) |
| 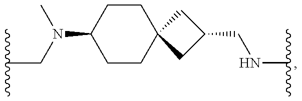 | (568) |
| 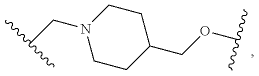 [SMILES: *CN1CCC(CO*)CC1] | (569) |
| 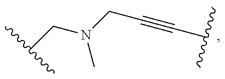 [SMILES: *C#CCN(C)C*] | (570) |
| 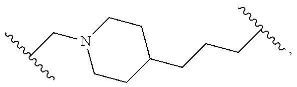 [SMILES: *CCCC1CCN(C*)CC1] | (571) |
| 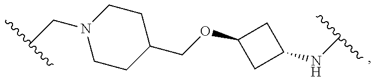 [SMILES: *CN1CCC(CO[C@H]2C[C@H](N*)C2)CC1] | (572) |
| 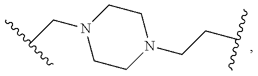 [SMILES: *CCN1CCN(C*)CC1] | (573) |
| 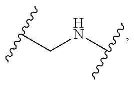 [SMILES: *CN*] | (574) |
| 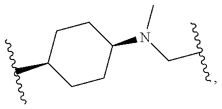 [SMILES: *CN(C)[C@H]1CC[C@@H](*)CC1] | (575) |
| 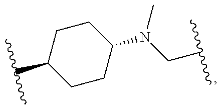 [SMILES: *CN(C)[C@H]1CC[C@H](*)CC1] | (576) |
| 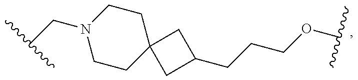 [SMILES: *CN1CCC2(CC1)CC(CCCO*)C2] | (577) |
| 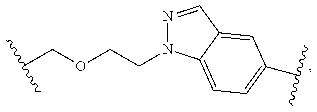 [SMILES: *COCCn1ncc2cc(*)ccc21] | (578) |
| 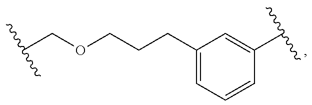 [SMILES: *COCCCc1cccc(*)c1] | (579) |
| 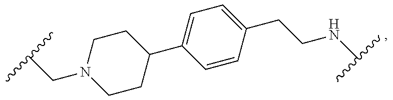 [SMILES: *CN1CCC(c2ccc(CCN*)cc2)CC1] | (580) |
| 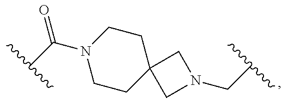 [SMILES: *CN1CC2(CCN(C(*)=O)CC2)C1] | (581) |
| 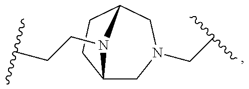 [SMILES: *CCN1[C@@H]2CC[C@H]1CN(C*)C2] | (582) |
| 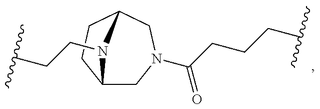 [SMILES: *CCCC(=O)N1C[C@H]2CC[C@@H](C1)N2CC*] | (583) |
| 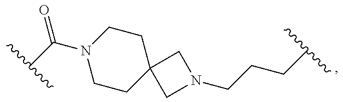 [SMILES: *CCCN1CC2(CCN(C(*)=O)CC2)C1] | (584) |
| 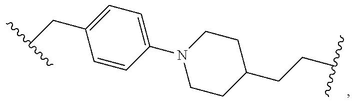 [SMILES: *CCC1CCN(c2ccc(C*)cc2)CC1] | (585) |
| 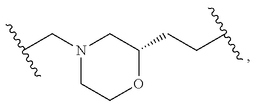 [SMILES: *CC[C@H]1CN(C*)CCO1] | (586) |
| 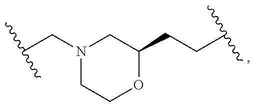 [SMILES: *CC[C@@H]1CN(C*)CCO1] | (587) |
| 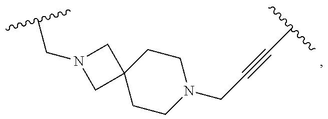 [SMILES: *C#CCN1CCC2(CC1)CN(C*)C2] | (588) |
| 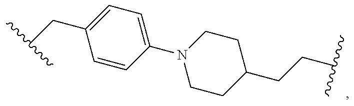 [SMILES: *CCC1CCN(c2ccc(C*)cc2)CC1] | (589) |
| 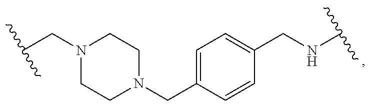 [SMILES: *CN1CCN(Cc2ccc(CN*)cc2)CC1] | (590) |
| 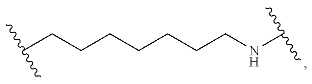 [SMILES: *CCCCCCCN*] | (591) |
| 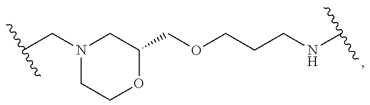 [SMILES: *CN1CCO[C@@H](COCCCN*)C1] | (592) |
| 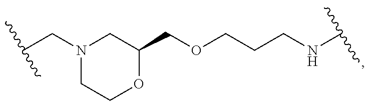 [SMILES: *CN1CCO[C@H](COCCCN*)C1] | (593) |
| 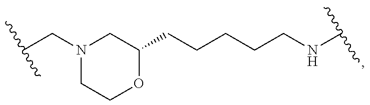 [SMILES: *CN1CCO[C@@H](CCCCCN*)C1] | (594) |
| 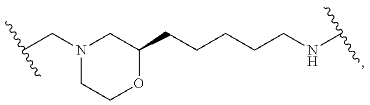 [SMILES: *CN1CCO[C@H](CCCCCN*)C1] | (595) |
| 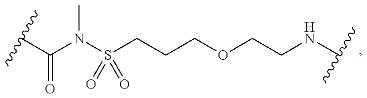 [SMILES: *NCCOCCCS(=O)(=O)N(C)C(*)=O] | (596) |
| 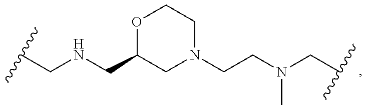 [SMILES: *CNC[C@@H]1CN(CCN(C)C*)CCO1] | (597) |
| 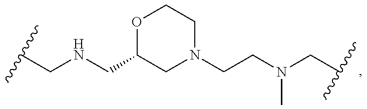 [SMILES: *CNC[C@H]1CN(CCN(C)C*)CCO1] | (598) |
| 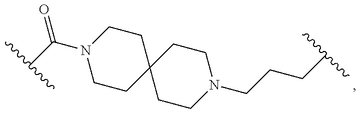 [SMILES: *CCCN1CCC2(CC1)CCN(C(*)=O)CC2] | (599) |
| 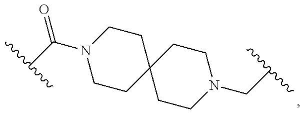 [SMILES: *CN1CCC2(CC1)CCN(C(*)=O)CC2] | (600) |
| 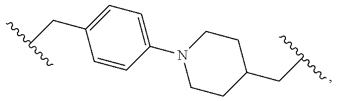 [SMILES: *Cc1ccc(N2CCC(C*)CC2)cc1] | (601) |
| 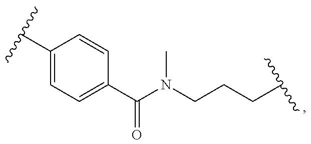 [SMILES: *CCCN(C)C(=O)c1ccc(*)cc1] | (602) |
| 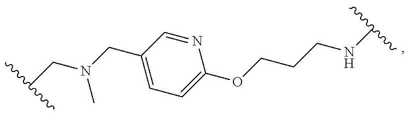 [SMILES: *CN(C)Cc1ccc(OCCCN*)nc1] | (603) |
| 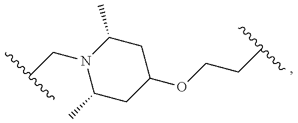 [SMILES: *CCOC1C[C@@H](C)N(C*)[C@@H](C)C1] | (604) |
| 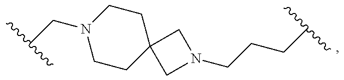 [SMILES: *CCCN1CC2(CCN(C*)CC2)C1] | (605) |
| 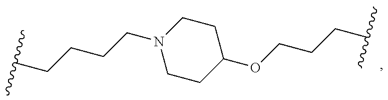 [SMILES: *CCCCN1CCC(OCCC*)CC1] | (606) |
| 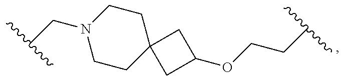 [SMILES: *CCOC1CC2(CCN(C*)CC2)C1] | (607) |
| 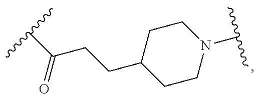 [SMILES: *C(=O)CCC1CCN(*)CC1] | (608) |
| 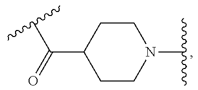 [SMILES: *C(=O)C1CCN(*)CC1] | (609) |
| 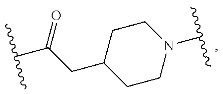 [SMILES: *C(=O)CC1CCN(*)CC1] | (610) |
| 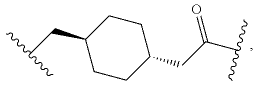 [SMILES: *C[C@H]1CC[C@H](CC(*)=O)CC1] | (611) |
| 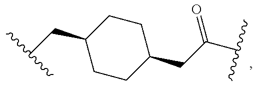 [SMILES: *C[C@H]1CC[C@@H](CC(*)=O)CC1] | (612) |
| 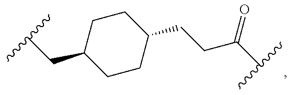 [SMILES: *C[C@H]1CC[C@H](CCC(*)=O)CC1] | (613) |
| 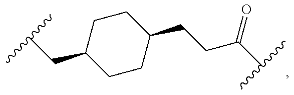 [SMILES: *C[C@H]1CC[C@@H](CCC(*)=O)CC1] | (614) |
| 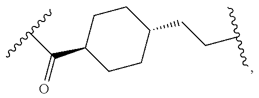 [SMILES: *CC[C@H]1CC[C@H](C(*)=O)CC1] | (615) |
| 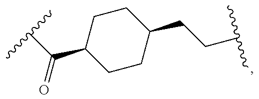 [SMILES: *CC[C@H]1CC[C@@H](C(*)=O)CC1] | (616) |
| 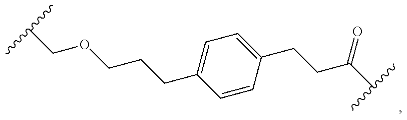 [SMILES: *COCCCc1ccc(CCC(*)=O)cc1] | (617) |
| 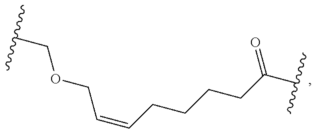 [SMILES: *COC/C=C\CCCCC(*)=O] | (618) |
| 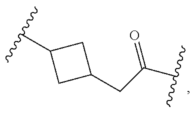 [SMILES: *C(=O)CC1CC(*)C1] | (619) |
| 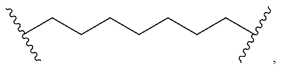 [SMILES: *CCCCCCC*] | (620) |
| 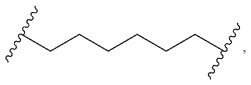 [SMILES: *CCCCCC*] | (621) |
| 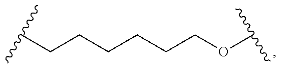 [SMILES: *CCCCCCO*] | (622) |
| 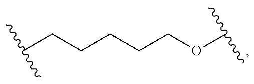 [SMILES: *CCCCCO*] | (623) |
| 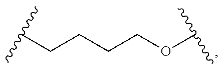 [SMILES: *CCCCO*] | (624) |
| 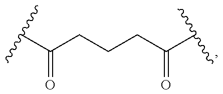 [SMILES: *C(=O)CCCC(*)=O] | (625) |
| 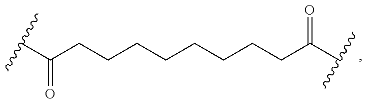 [SMILES: *C(=O)CCCCCCCCC(*)=O] | (626) |
| 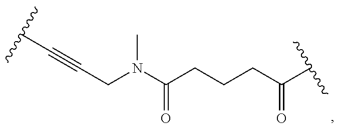 [SMILES: *C#CCN(C)C(=O)CCCC(*)=O] | (627) |
| 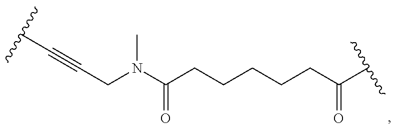 [SMILES: *C#CCN(C)C(=O)CCCCCC(*)=O] | (628) |
| 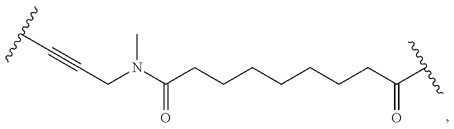 [SMILES: *C#CCN(C)C(=O)CCCCCCCC(*)=O] | (629) |
| 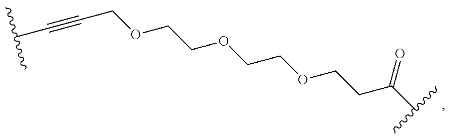 [SMILES: *C#CCOCCOCCOCCC(*)=O] | (630) |
| 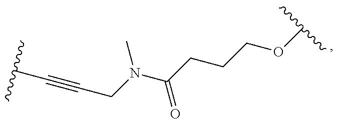 [SMILES: *C#CCN(C)C(=O)CCCO*] | (631) |
| 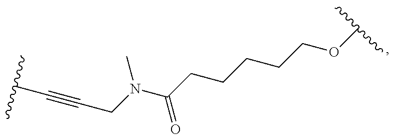 [SMILES: *C#CCN(C)C(=O)CCCCCO*] | (632) |
| 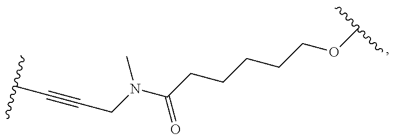 [SMILES: *C#CCN(C)C(=O)CCCCCO*] | (633) |
| 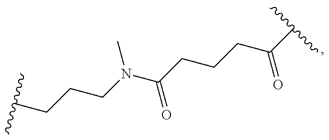 [SMILES: *CCCN(C)C(=O)CCCC(*)=O] | (634) |
| 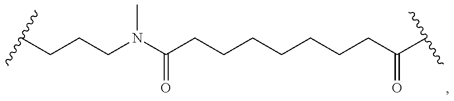 [SMILES: *CCCN(C)C(=O)CCCCCCCC(*)=O] | (635) |
| 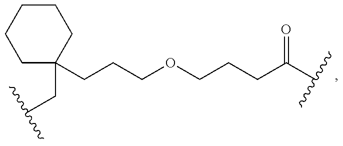 [SMILES: *CC1(CCCOCCCC(*)=O)CCCCC1] | (636) |
| 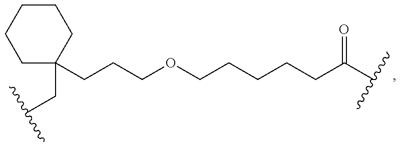 [SMILES: *CC1(CCCOCCCCCC(*)=O)CCCCC1] | (637) |
| 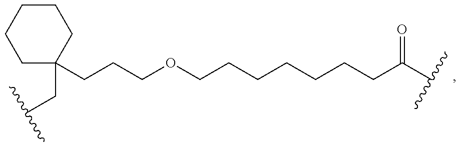 [SMILES: *CC1(CCCOCCCCCCCC(*)=O)CCCCC1] | (638) |
| 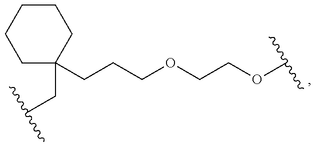 [SMILES: *CC1(CCCOCCO*)CCCCC1] | (639) |
| 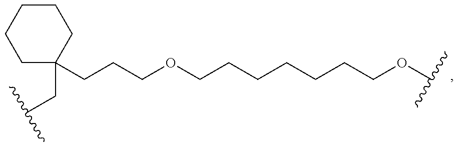 [SMILES: *CC1(CCCOCCCCCCCO*)CCCCC1] | (640) |
| 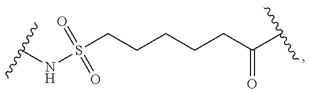 [SMILES: *NS(=O)(=O)CCCCCC(*)=O] | (641) |
| 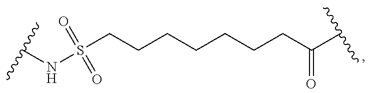 [SMILES: *NS(=O)(=O)CCCCCCCC(*)=O] | (642) |
| 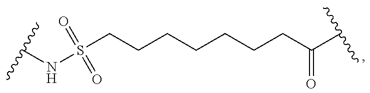 [SMILES: *NS(=O)(=O)CCCCCCCC(*)=O] | (643) |
| 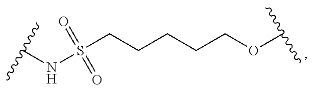 [SMILES: *NS(=O)(=O)CCCCCO*] | (644) |
| 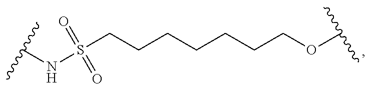 [SMILES: *NS(=O)(=O)CCCCCCCO*] | (645) |
| 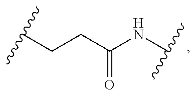 [SMILES: *CCC(=O)N*] | (646) |
| 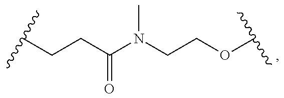 [SMILES: *CCC(=O)N(C)CCO*] | (647) |
| 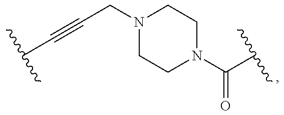 [SMILES: *C#CCN1CCN(C(*)=O)CC1] | (648) |
| 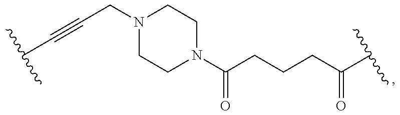 [SMILES: *C#CCN1CCN(C(=O)CCCC(*)=O)CC1] | (649) |
| 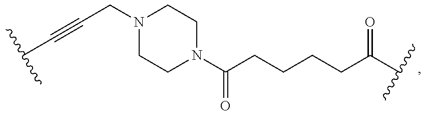 [SMILES: *C#CCN1CCN(C(=O)CCCCC(*)=O)CC1] | (650) |
| 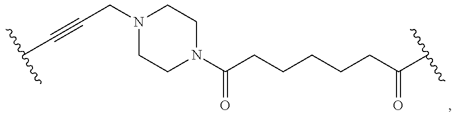 [SMILES: *C#CCN1CCN(C(=O)CCCCCC(*)=O)CC1] | (651) |
| 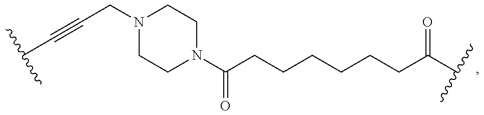 [SMILES: *C#CCN1CCN(C(=O)CCCCCCC(*)=O)CC1] | (652) |
| 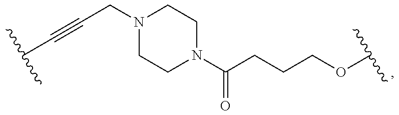 [SMILES: *C#CCN1CCN(C(=O)CCCO*)CC1] | (653) |
| 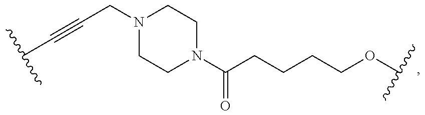 [SMILES: *C#CCN1CCN(C(=O)CCCCO*)CC1] | (654) |
| 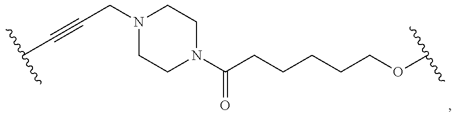 [SMILES: *C#CCN1CCN(C(=O)CCCCCO*)CC1] | (655) |
| 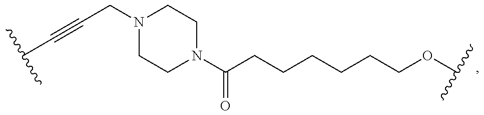 [SMILES: *C#CCN1CCN(C(=O)CCCCCCO*)CC1] | (656) |
| 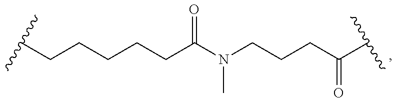 [SMILES: *CCCCCC(=O)N(C)CCCC(*)=O] | (657) |
| 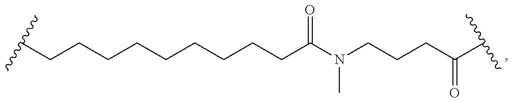 [SMILES: *CCCCCCCCCC(=O)N(C)CCCC(*)=O] | (658) |
| 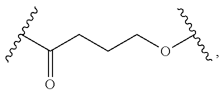 [SMILES: *OCCCC(*)=O] | (659) |
| 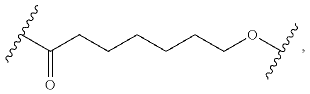 [SMILES: *OCCCCCCC(*)=O] | (660) |
| 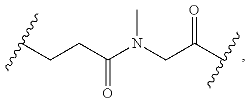 [SMILES: *CCC(=O)N(C)CC(*)=O] | (661) |
| 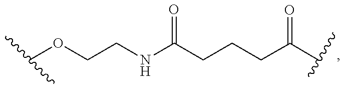 [SMILES: *OCCNC(=O)CCCC(*)=O] | (662) |
| 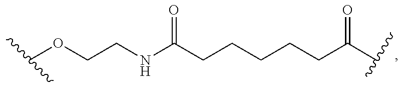 [SMILES: *OCCNC(=O)CCCCCC(*)=O] | (663) |
| 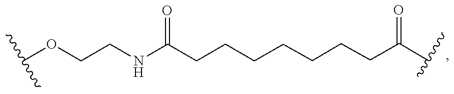 [SMILES: *OCCNC(=O)CCCCCCCC(*)=O] | (664) |
| 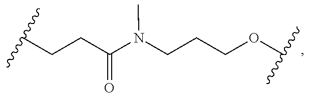 [SMILES: *CCC(=O)N(C)CCCO*] | (665) |
| 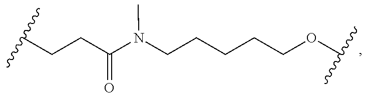 [SMILES: *CCC(=O)N(C)CCCCCO*] | (666) |
| 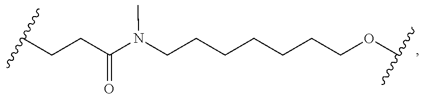 [SMILES: *CCC(=O)N(C)CCCCCCCO*] | (667) |
| 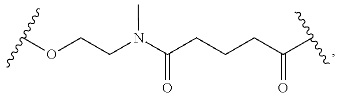 [SMILES: *OCCN(C)C(=O)CCCC(*)=O] | (668) |
| 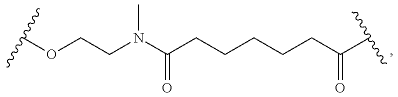 [SMILES: *OCCN(C)C(=O)CCCCCC(*)=O] | (669) |
| 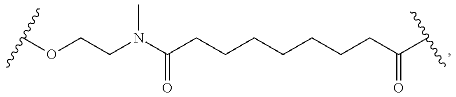 [SMILES: *OCCN(C)C(=O)CCCCCCCC(*)=O] | (670) |
| 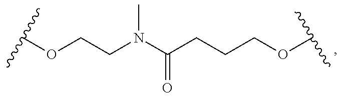 [SMILES: *OCCCC(=O)N(C)CCO*] | (671) |
| 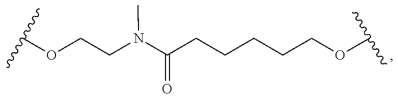 [SMILES: *OCCCCCC(=O)N(C)CCO*] | (672) |
| 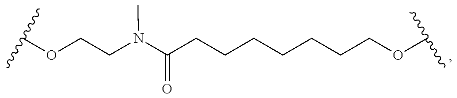 [SMILES: *OCCCCCCCC(=O)N(C)CCO*] | (673) |
| 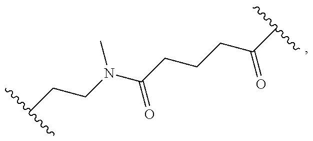 [SMILES: *CCN(C)C(=O)CCCC(*)=O] | (674) |
| 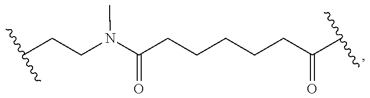 [SMILES: *CCN(C)C(=O)CCCCCC(*)=O] | (675) |
| 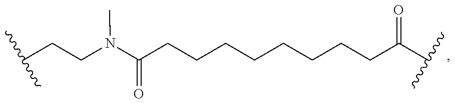 [SMILES: *CCN(C)C(=O)CCCCCCCCC(*)=O] | (676) |
| 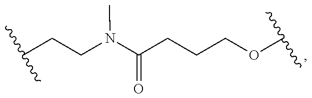 [SMILES: *CCN(C)C(=O)CCCO*] | (677) |
| 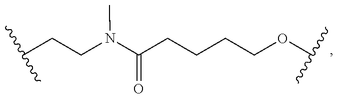 [SMILES: *CCN(C)C(=O)CCCCO*] | (678) |
| 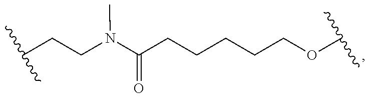 [SMILES: *CCN(C)C(=O)CCCCCO*] | (679) |
| 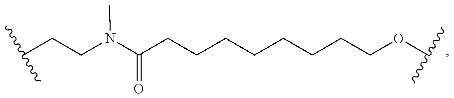 [SMILES: *CCN(C)C(=O)CCCCCCCCO*] | (680) |
| 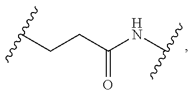 [SMILES: *CCC(=O)N*] | (681) |
| 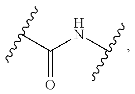 [SMILES: *NC(*)=O] | (682) |
| 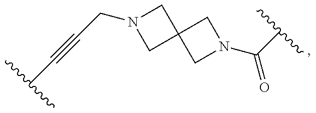 [SMILES: *C#CCN1CC2(C1)CN(C(*)=O)C2] | (683) |
| 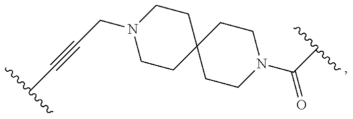 [SMILES: *C#CCN1CCC2(CC1)CCN(C(*)=O)CC2] | (684) |
| 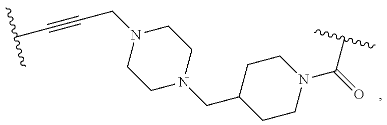 [SMILES: *C#CCN1CCN(CC2CCN(C(*)=O)CC2)CC1] | (685) |
| 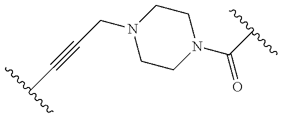 [SMILES: *C#CCN1CCN(C(*)=O)CC1] | (686) |

In some embodiments, the present invention provides a compound having TBM described and disclosed herein, LBM set forth in Table A above, and a linker set forth in Table B above, or a pharmaceutically acceptable salt thereof.

Exemplary compounds of the invention are set forth in Table 1 below.

TABLE 1
Exemplary Compounds

| Com- | |
|---|---|
| pound | |
| ID | Compound Structure |
| K101 | 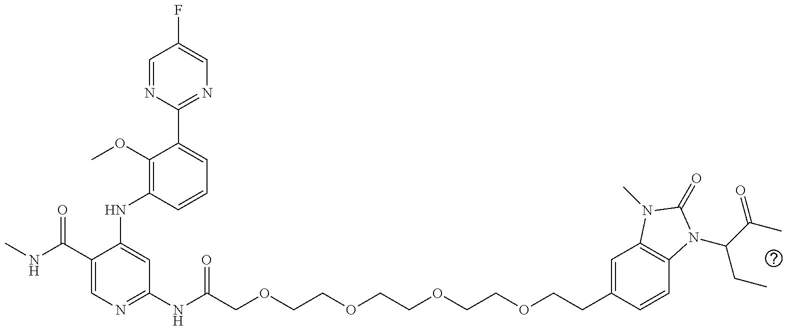 [SMILES: CCC(C(C)=O)n1c(=O)n(C)c2cc(CCOCCOCCOCCOCC(=O)Nc3cc(Nc4cccc(-c5ncc(F)cn5)c4OC)c(C(=O)NC)cn3)ccc21] |
| K102 | 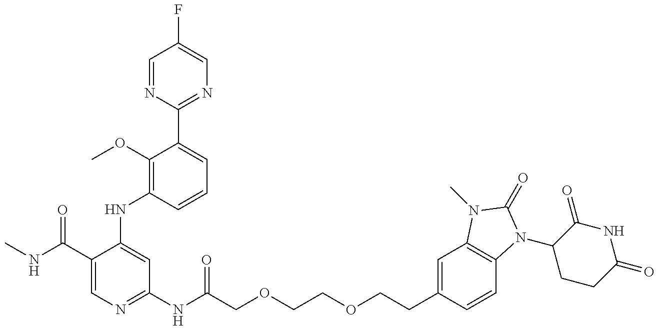 [SMILES: CNC(=O)c1cnc(NC(=O)COCCOCCc2ccc3c(c2)n(C)c(=O)n3C2CCC(=O)NC2=O)cc1Nc1cccc(-c2ncc(F)cn2)c1OC] |
| K103 | 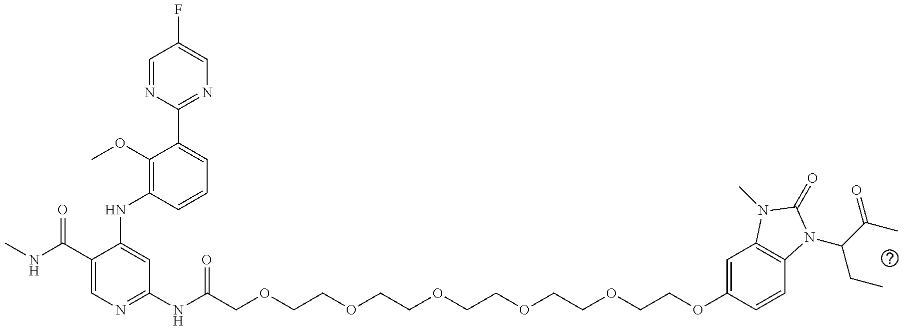 [SMILES: CCC(C(C)=O)n1c(=O)n(C)c2cc(OCCOCCOCCOCCOCCOCC(=O)Nc3cc(Nc4cccc(-c5ncc(F)cn5)c4OC)c(C(=O)NC)cn3)ccc21] |
| K104 | 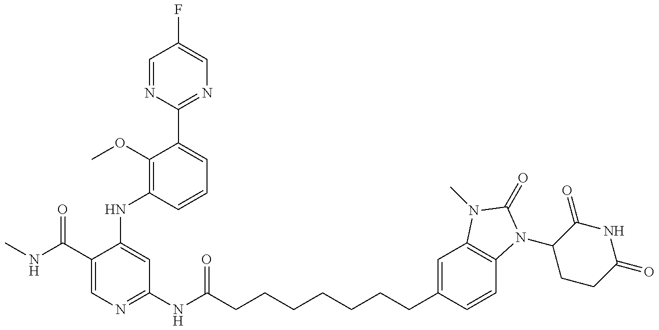 [SMILES: CNC(=O)c1cnc(NC(=O)CCCCCCCc2ccc3c(c2)n(C)c(=O)n3C2CCC(=O)NC2=O)cc1Nc1cccc(-c2ncc(F)cn2)c1OC] |
| K105 | 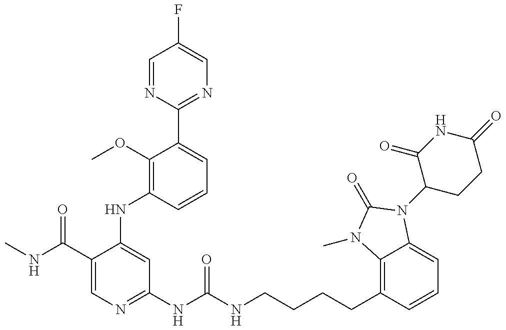 [SMILES: CNC(=O)c1cnc(NC(=O)NCCCCc2cccc3c2n(C)c(=O)n3C2CCC(=O)NC2=O)cc1Nc1cccc(-c2ncc(F)cn2)c1OC] |
| K106 | 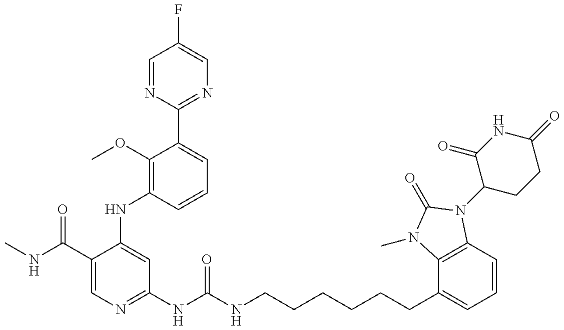 [SMILES: CNC(=O)c1cnc(NC(=O)NCCCCCCc2cccc3c2n(C)c(=O)n3C2CCC(=O)NC2=O)cc1Nc1cccc(-c2ncc(F)cn2)c1OC] |
| K107 | 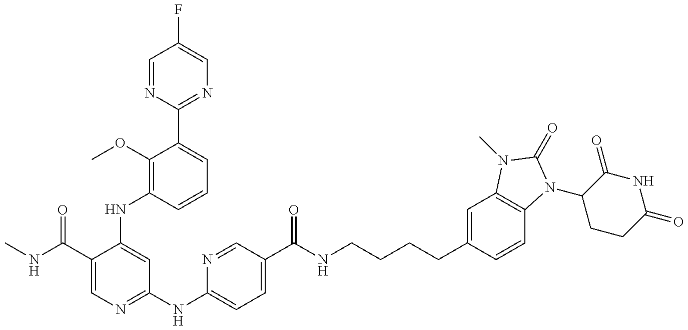 [SMILES: CNC(=O)c1cnc(Nc2ccc(C(=O)NCCCCc3ccc4c(c3)n(C)c(=O)n4C3CCC(=O)NC3=O)cn2)cc1Nc1cccc(-c2ncc(F)cn2)c1OC] |
| K108 | 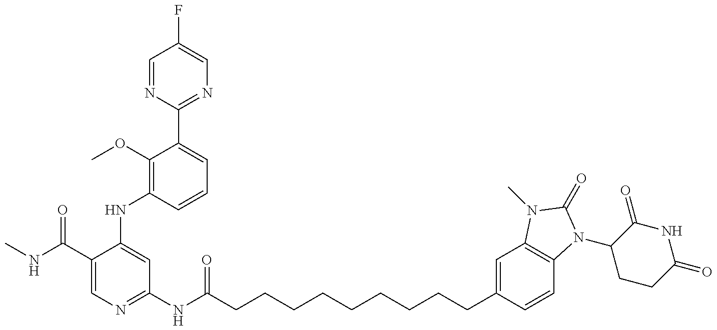 [SMILES: CNC(=O)c1cnc(NC(=O)CCCCCCCCCc2ccc3c(c2)n(C)c(=O)n3C2CCC(=O)NC2=O)cc1Nc1cccc(-c2ncc(F)cn2)c1OC] |
| K109 | 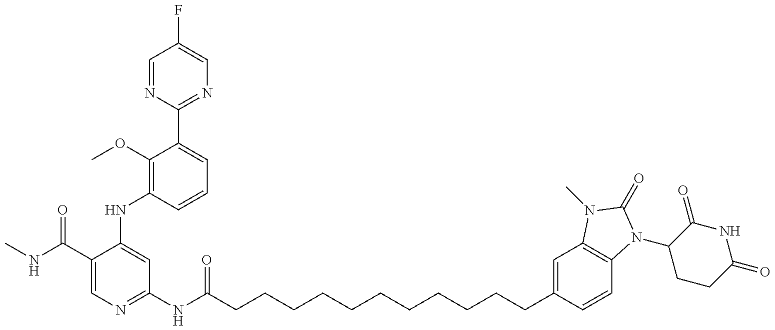 [SMILES: CNC(=O)c1cnc(NC(=O)CCCCCCCCCCCc2ccc3c(c2)n(C)c(=O)n3C2CCC(=O)NC2=O)cc1Nc1cccc(-c2ncc(F)cn2)c1OC] |
| K110 | 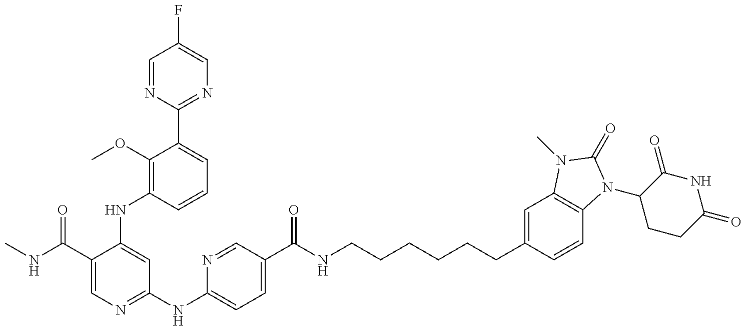 [SMILES: CNC(=O)c1cnc(Nc2ccc(C(=O)NCCCCCCc3ccc4c(c3)n(C)c(=O)n4C3CCC(=O)NC3=O)cn2)cc1Nc1cccc(-c2ncc(F)cn2)c1OC] |
| K111 | 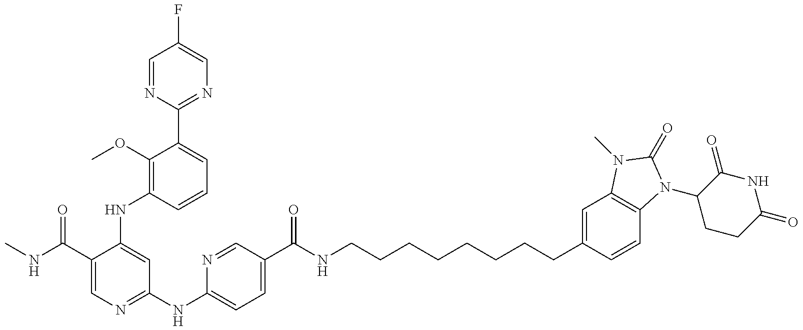 [SMILES: CNC(=O)c1cnc(Nc2ccc(C(=O)NCCCCCCCCc3ccc4c(c3)n(C)c(=O)n4C3CCC(=O)NC3=O)cn2)cc1Nc1cccc(-c2ncc(F)cn2)c1OC] |
| K112 | 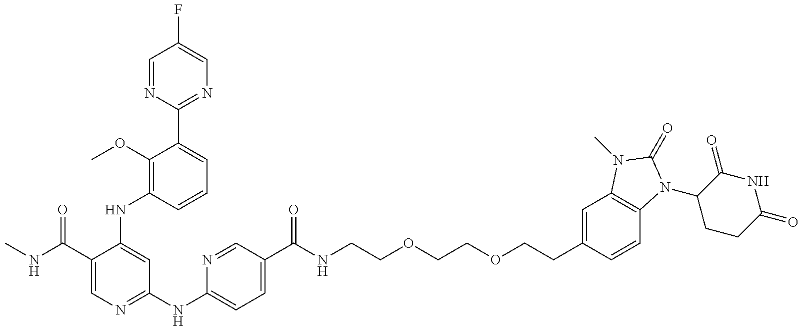 [SMILES: CNC(=O)c1cnc(Nc2ccc(C(=O)NCCOCCOCCc3ccc4c(c3)n(C)c(=O)n4C3CCC(=O)NC3=O)cn2)cc1Nc1cccc(-c2ncc(F)cn2)c1OC] |
| K113 | 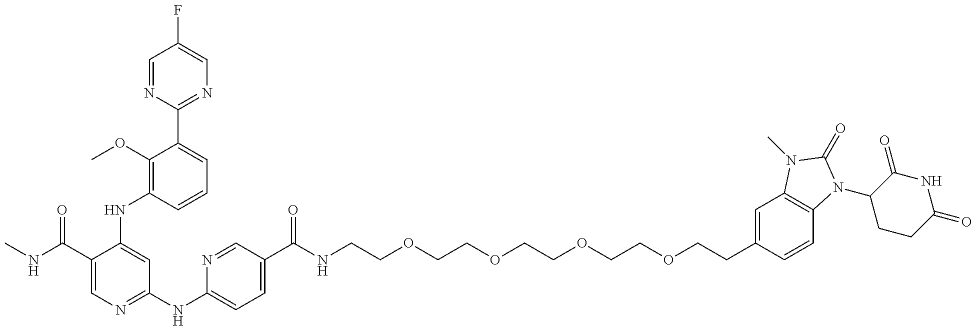 [SMILES: CNC(=O)c1cnc(Nc2ccc(C(=O)NCCOCCOCCOCCOCCc3ccc4c(c3)n(C)c(=O)n4C3CCC(=O)NC3=O)cn2)cc1Nc1cccc(-c2ncc(F)cn2)c1OC] |
| K114 | 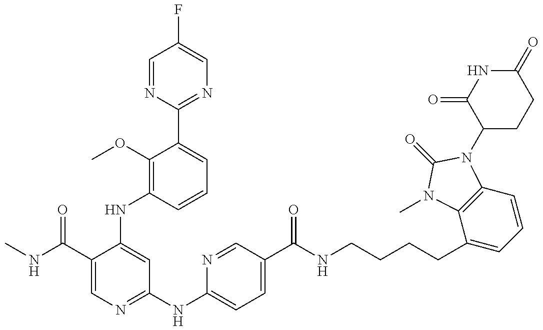 [SMILES: CNC(=O)c1cnc(Nc2ccc(C(=O)NCCCCc3cccc4c3n(C)c(=O)n4C3CCC(=O)NC3=O)cn2)cc1Nc1cccc(-c2ncc(F)cn2)c1OC] |
| K115 | 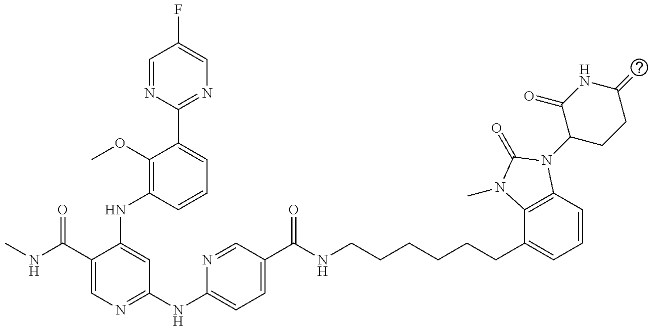 |
| K116 | 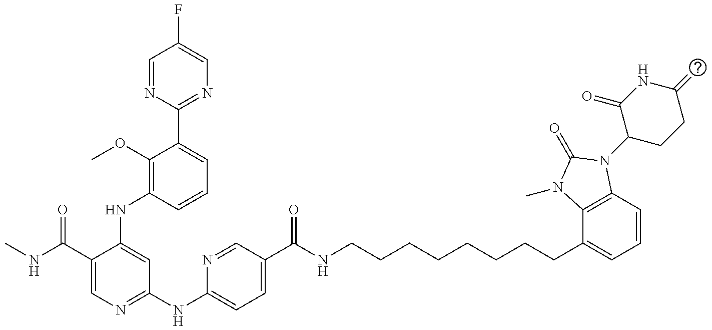 |
| K117 | 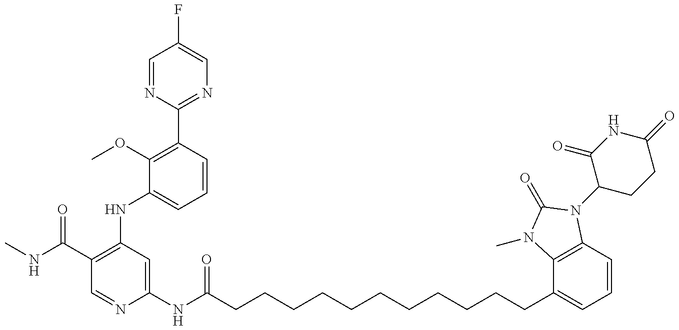 [SMILES: CNC(=O)c1cnc(NC(=O)CCCCCCCCCCCc2cccc3c2n(C)c(=O)n3C2CCC(=O)NC2=O)cc1Nc1cccc(-c2ncc(F)cn2)c1OC] |
| K118 | 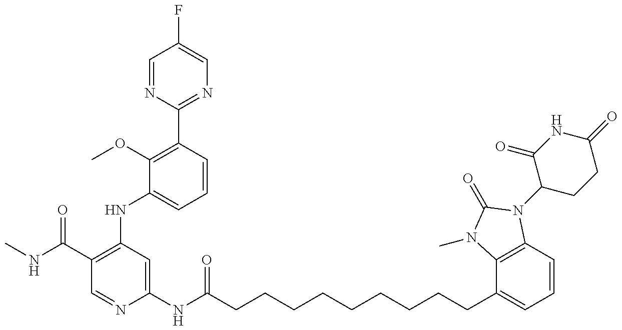 [SMILES: CNC(=O)c1cnc(NC(=O)CCCCCCCCCc2cccc3c2n(C)c(=O)n3C2CCC(=O)NC2=O)cc1Nc1cccc(-c2ncc(F)cn2)c1OC] |
| K119 | 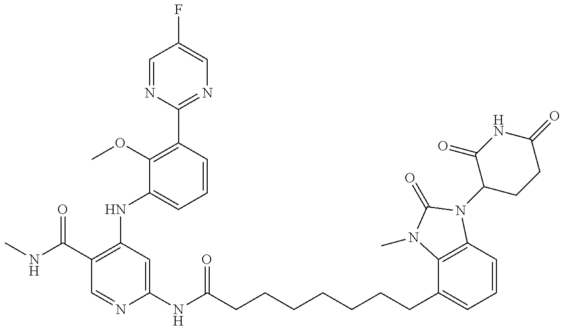 [SMILES: CNC(=O)c1cnc(NC(=O)CCCCCCCc2cccc3c2n(C)c(=O)n3C2CCC(=O)NC2=O)cc1Nc1cccc(-c2ncc(F)cn2)c1OC] |
| K120 | 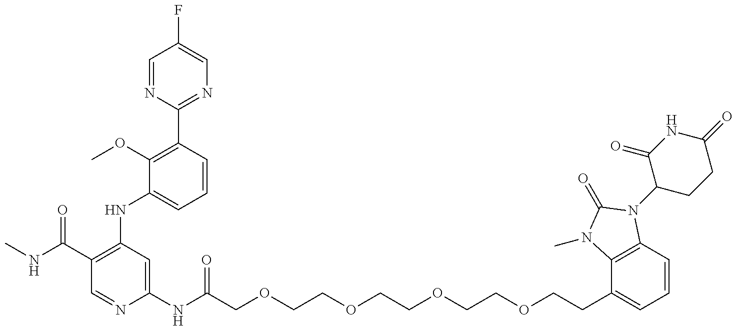 [SMILES: CNC(=O)c1cnc(NC(=O)COCCOCCOCCOCCc2cccc3c2n(C)c(=O)n3C2CCC(=O)NC2=O)cc1Nc1cccc(-c2ncc(F)cn2)c1OC] |
| K121 | 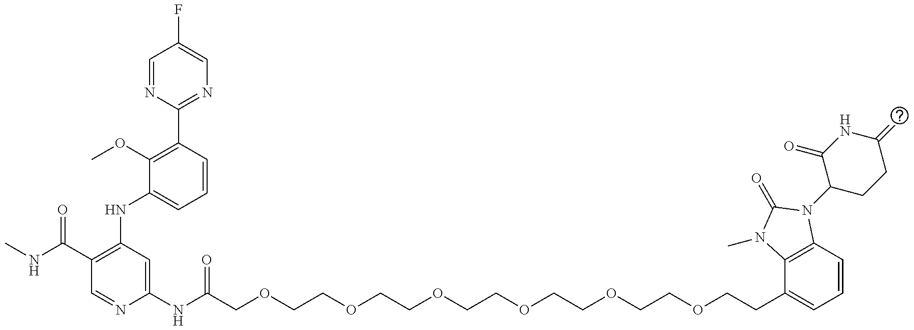 |
| K122 | 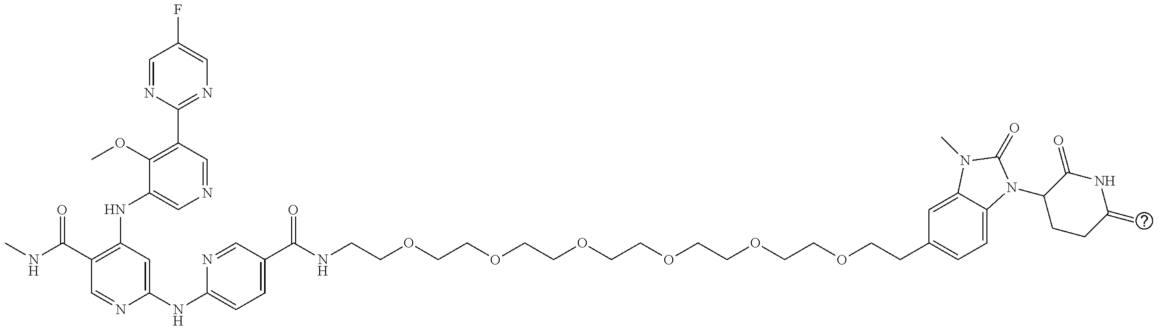 |
| K123 | 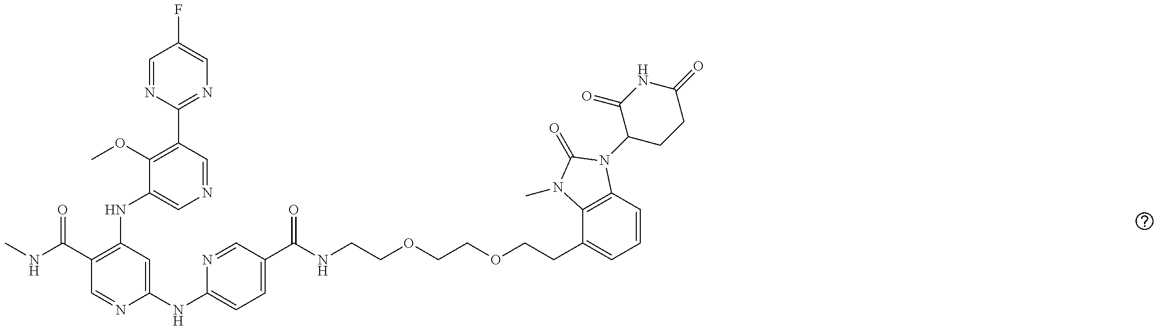 [SMILES: CNC(=O)c1cnc(Nc2ccc(C(=O)NCCOCCOCCc3cccc4c3n(C)c(=O)n4C3CCC(=O)NC3=O)cn2)cc1Nc1cncc(-c2ncc(F)cn2)c1OC] |
| K124 | 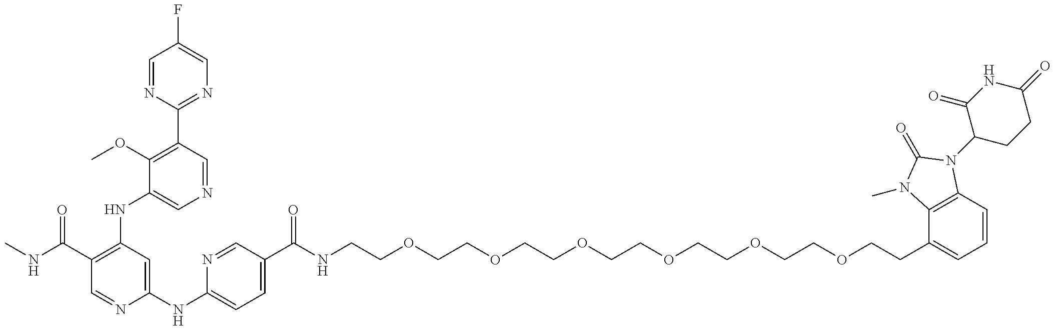 [SMILES: CNC(=O)c1cnc(Nc2ccc(C(=O)NCCOCCOCCOCCOCCOCCOCCc3cccc4c3n(C)c(=O)n4C3CCC(=O)NC3=O)cn2)cc1Nc1cncc(-c2ncc(F)cn2)c1OC] |
| K125 | 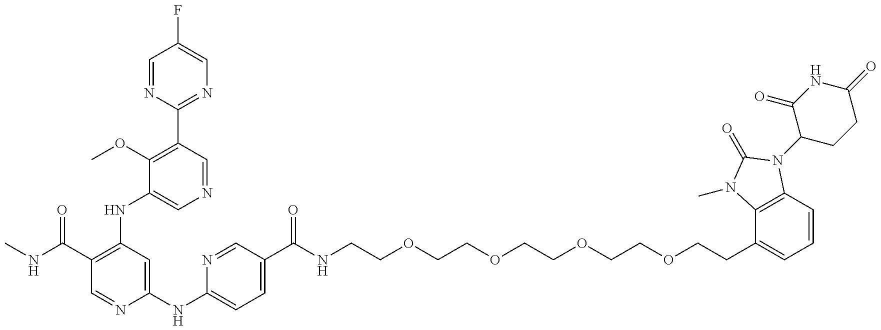 [SMILES: CNC(=O)c1cnc(Nc2ccc(C(=O)NCCOCCOCCOCCOCCc3cccc4c3n(C)c(=O)n4C3CCC(=O)NC3=O)cn2)cc1Nc1cncc(-c2ncc(F)cn2)c1OC] |
| K126 | 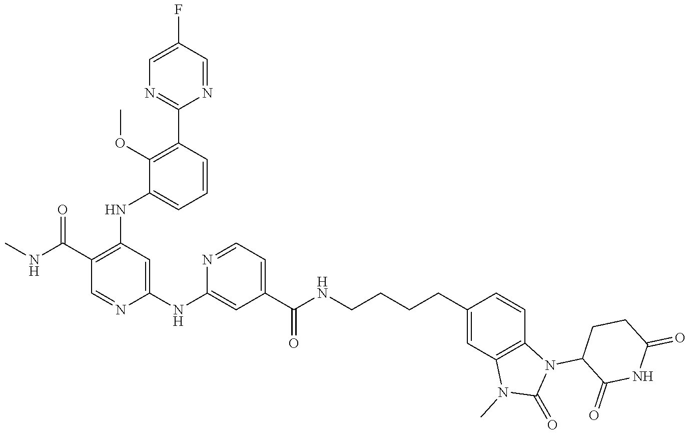 [SMILES: CNC(=O)c1cnc(Nc2cc(C(=O)NCCCCc3ccc4c(c3)n(C)c(=O)n4C3CCC(=O)NC3=O)ccn2)cc1Nc1cccc(-c2ncc(F)cn2)c1OC] |
| K127 | 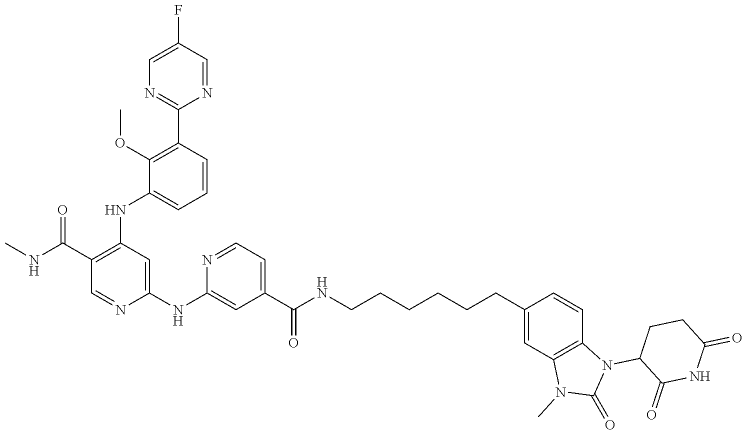 [SMILES: CNC(=O)c1cnc(Nc2cc(C(=O)NCCCCCCc3ccc4c(c3)n(C)c(=O)n4C3CCC(=O)NC3=O)ccn2)cc1Nc1cccc(-c2ncc(F)cn2)c1OC] |
| K128 | 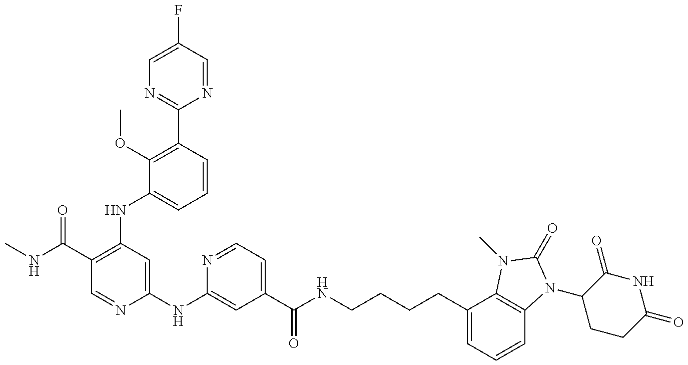 [SMILES: CNC(=O)c1cnc(Nc2cc(C(=O)NCCCCc3cccc4c3n(C)c(=O)n4C3CCC(=O)NC3=O)ccn2)cc1Nc1cccc(-c2ncc(F)cn2)c1OC] |
| K129 | 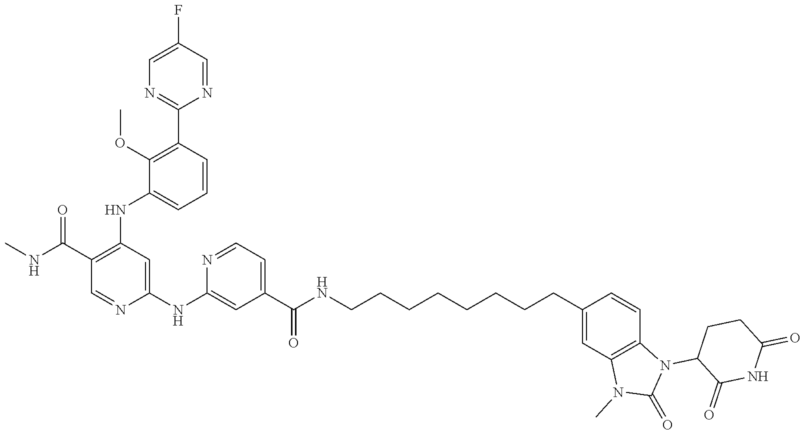 [SMILES: CNC(=O)c1cnc(Nc2cc(C(=O)NCCCCCCCCc3ccc4c(c3)n(C)c(=O)n4C3CCC(=O)NC3=O)ccn2)cc1Nc1cccc(-c2ncc(F)cn2)c1OC] |
| K130 | 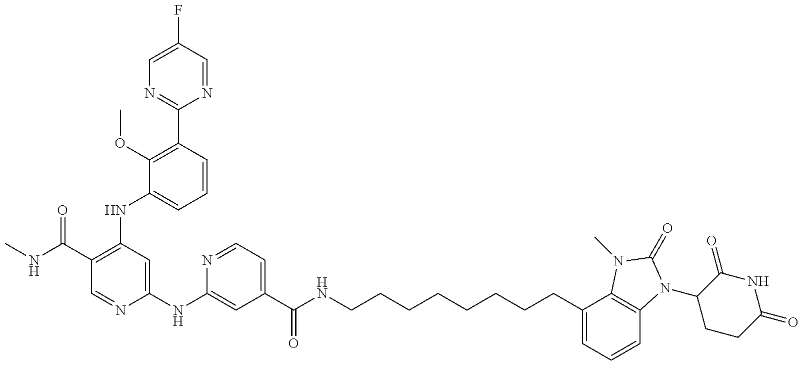 [SMILES: CNC(=O)c1cnc(Nc2cc(C(=O)NCCCCCCCCc3cccc4c3n(C)c(=O)n4C3CCC(=O)NC3=O)ccn2)cc1Nc1cccc(-c2ncc(F)cn2)c1OC] |
| K131 | 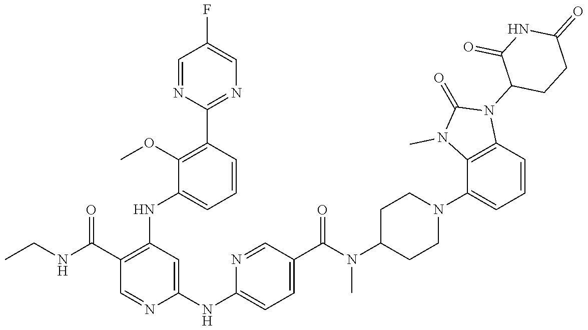 [SMILES: CCNC(=O)c1cnc(Nc2ccc(C(=O)N(C)C3CCN(c4cccc5c4n(C)c(=O)n5C4CCC(=O)NC4=O)CC3)cn2)cc1Nc1cccc(-c2ncc(F)cn2)c1OC] |
| K132 | 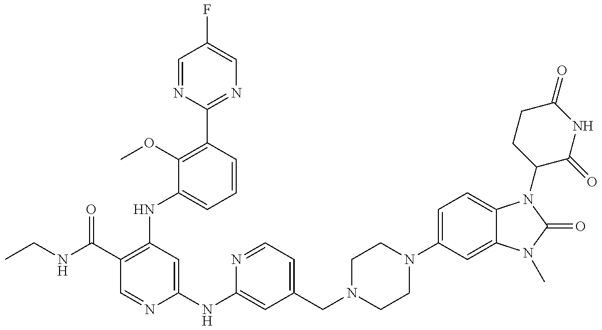 [SMILES: CCNC(=O)c1cnc(Nc2cc(CN3CCN(c4ccc5c(c4)n(C)c(=O)n5C4CCC(=O)NC4=O)CC3)ccn2)cc1Nc1cccc(-c2ncc(F)cn2)c1OC] |
| K133 | 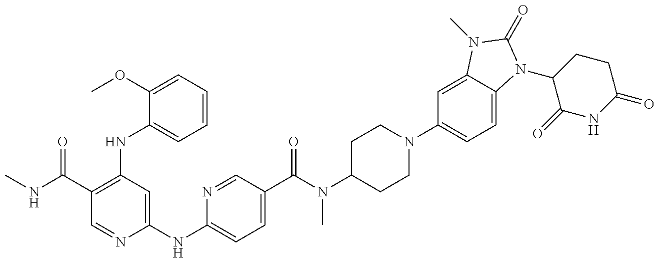 [SMILES: CNC(=O)c1cnc(Nc2ccc(C(=O)N(C)C3CCN(c4ccc5c(c4)n(C)c(=O)n5C4CCC(=O)NC4=O)CC3)cn2)cc1Nc1ccccc1OC] |
| K134 | 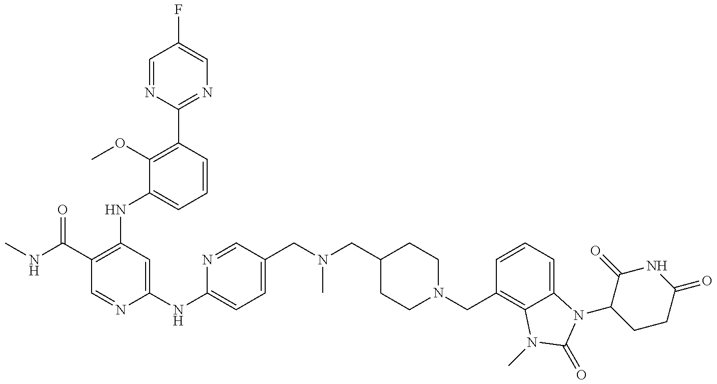 [SMILES: CNC(=O)c1cnc(Nc2ccc(CN(C)CC3CCN(Cc4cccc5c4n(C)c(=O)n5C4CCC(=O)NC4=O)CC3)cn2)cc1Nc1cccc(-c2ncc(F)cn2)c1OC] |
| K135 | 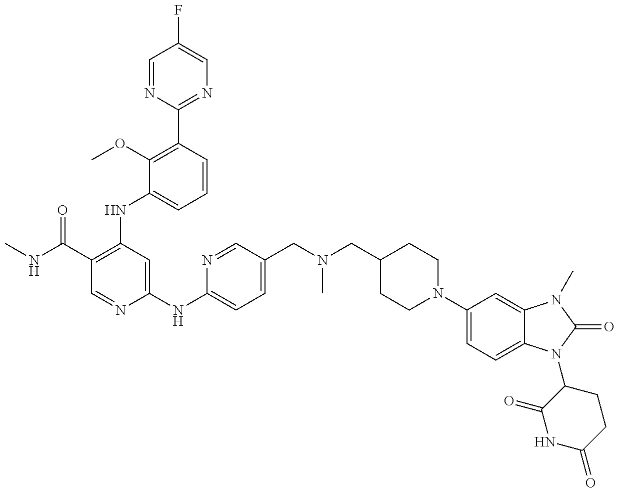 [SMILES: CNC(=O)c1cnc(Nc2ccc(CN(C)CC3CCN(c4ccc5c(c4)n(C)c(=O)n5C4CCC(=O)NC4=O)CC3)cn2)cc1Nc1cccc(-c2ncc(F)cn2)c1OC] |
| K136 | 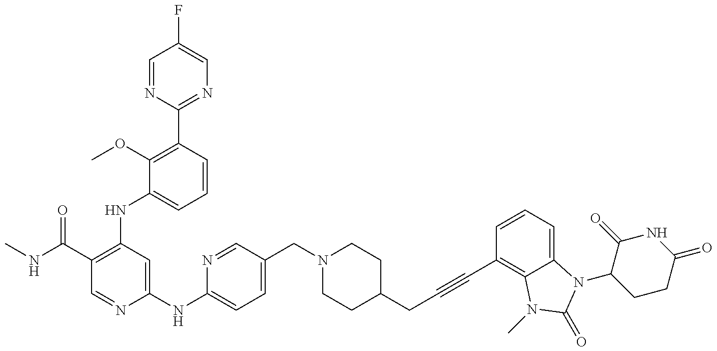 [SMILES: CNC(=O)c1cnc(Nc2ccc(CN3CCC(CC#Cc4cccc5c4n(C)c(=O)n5C4CCC(=O)NC4=O)CC3)cn2)cc1Nc1cccc(-c2ncc(F)cn2)c1OC] |
| K137 | 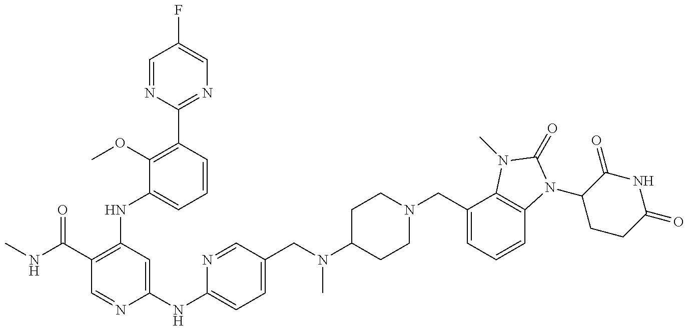 [SMILES: CNC(=O)c1cnc(Nc2ccc(CN(C)C3CCN(Cc4cccc5c4n(C)c(=O)n5C4CCC(=O)NC4=O)CC3)cn2)cc1Nc1cccc(-c2ncc(F)cn2)c1OC] |
| K138 | 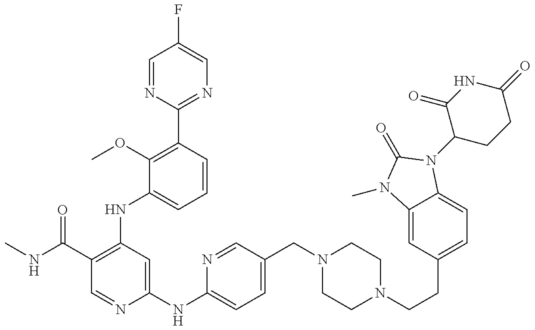 [SMILES: CNC(=O)c1cnc(Nc2ccc(CN3CCN(CCc4ccc5c(c4)n(C)c(=O)n5C4CCC(=O)NC4=O)CC3)cn2)cc1Nc1cccc(-c2ncc(F)cn2)c1OC] |
| K139 | 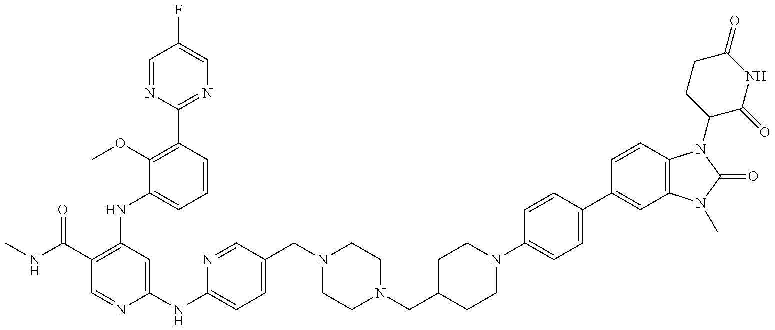 [SMILES: CNC(=O)c1cnc(Nc2ccc(CN3CCN(CC4CCN(c5ccc(-c6ccc7c(c6)n(C)c(=O)n7C6CCC(=O)NC6=O)cc5)CC4)CC3)cn2)cc1Nc1cccc(-c2ncc(F)cn2)c1OC] |
| K140 | 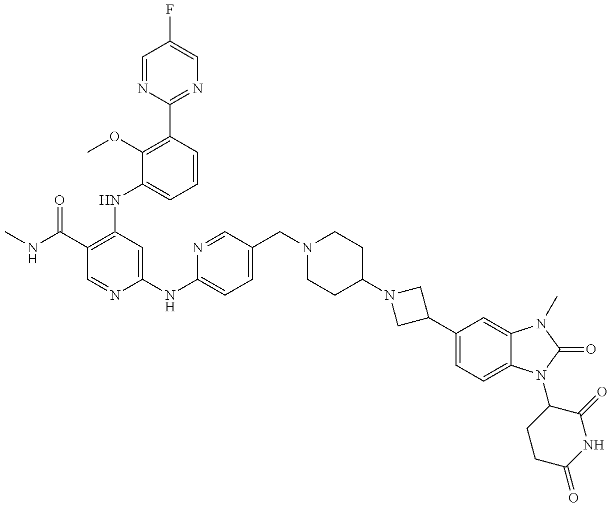 [SMILES: CNC(=O)c1cnc(Nc2ccc(CN3CCC(N4CC(c5ccc6c(c5)n(C)c(=O)n6C5CCC(=O)NC5=O)C4)CC3)cn2)cc1Nc1cccc(-c2ncc(F)cn2)c1OC] |
| K141 | 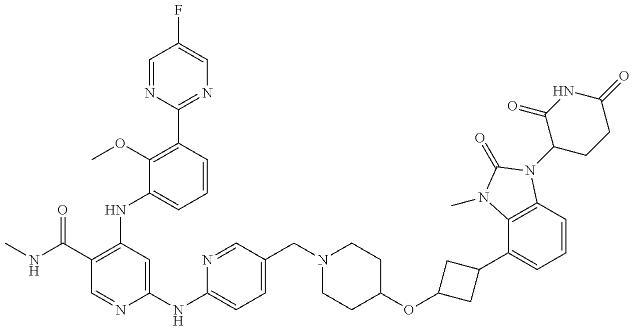 [SMILES: CNC(=O)c1cnc(Nc2ccc(CN3CCC(OC4CC(c5cccc6c5n(C)c(=O)n6C5CCC(=O)NC5=O)C4)CC3)cn2)cc1Nc1cccc(-c2ncc(F)cn2)c1OC] |
| K142 | 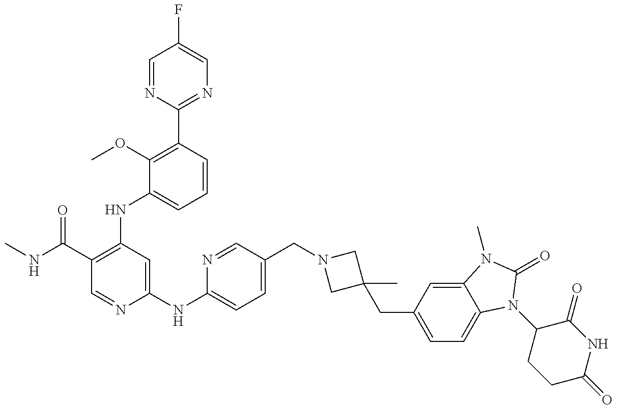 [SMILES: CNC(=O)c1cnc(Nc2ccc(CN3CC(C)(Cc4ccc5c(c4)n(C)c(=O)n5C4CCC(=O)NC4=O)C3)cn2)cc1Nc1cccc(-c2ncc(F)cn2)c1OC] |
| K143 | 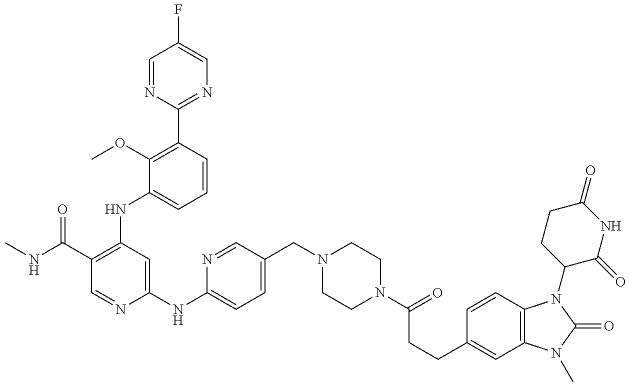 [SMILES: CNC(=O)c1cnc(Nc2ccc(CN3CCN(C(=O)CCc4ccc5c(c4)n(C)c(=O)n5C4CCC(=O)NC4=O)CC3)cn2)cc1Nc1cccc(-c2ncc(F)cn2)c1OC] |
| K144 | 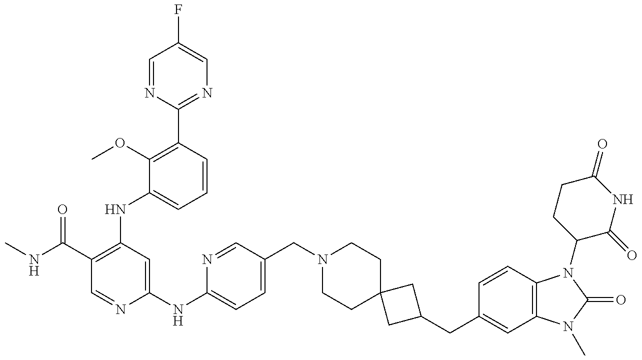 [SMILES: CNC(=O)c1cnc(Nc2ccc(CN3CCC4(CC3)CC(Cc3ccc5c(c3)n(C)c(=O)n5C3CCC(=O)NC3=O)C4)cn2)cc1Nc1cccc(-c2ncc(F)cn2)c1OC] |
| K145 | 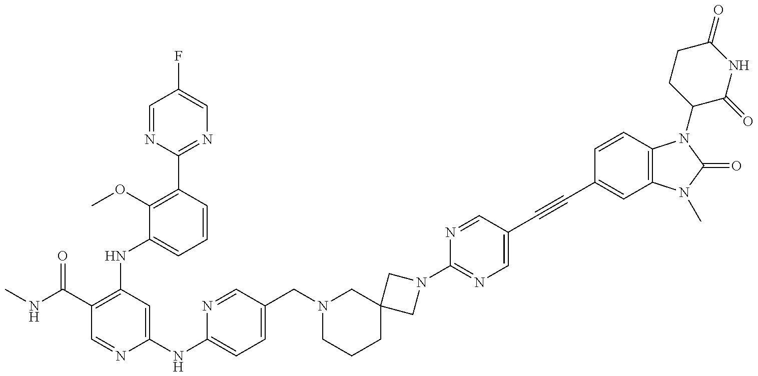 [SMILES: CNC(=O)c1cnc(Nc2ccc(CN3CCCC4(C3)CN(c3ncc(C#Cc5ccc6c(c5)n(C)c(=O)n6C5CCC(=O)NC5=O)cn3)C4)cn2)cc1Nc1cccc(-c2ncc(F)cn2)c1OC] |
| K146 | 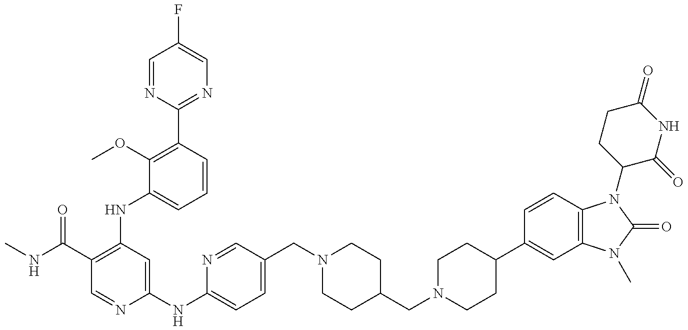 [SMILES: CNC(=O)c1cnc(Nc2ccc(CN3CCC(CN4CCC(c5ccc6c(c5)n(C)c(=O)n6C5CCC(=O)NC5=O)CC4)CC3)cn2)cc1Nc1cccc(-c2ncc(F)cn2)c1OC] |
| K147 | 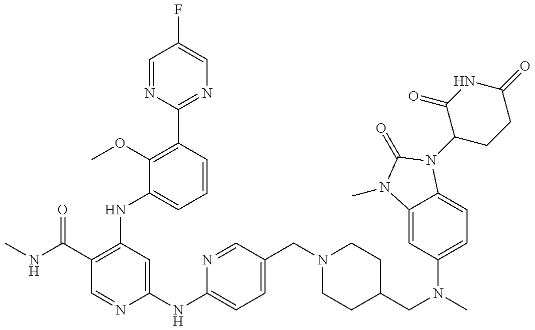 [SMILES: CNC(=O)c1cnc(Nc2ccc(CN3CCC(CN(C)c4ccc5c(c4)n(C)c(=O)n5C4CCC(=O)NC4=O)CC3)cn2)cc1Nc1cccc(-c2ncc(F)cn2)c1OC] |
| K148 | 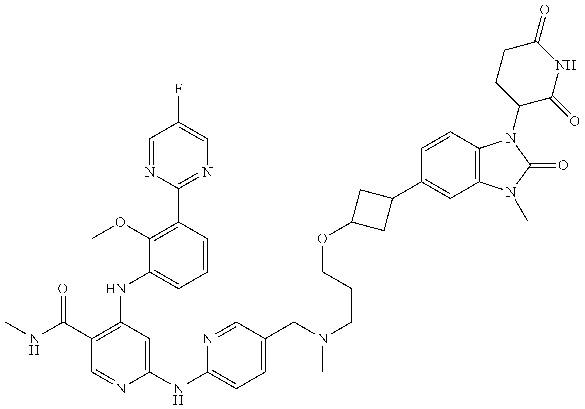 [SMILES: CNC(=O)c1cnc(Nc2ccc(CN(C)CCCOC3CC(c4ccc5c(c4)n(C)c(=O)n5C4CCC(=O)NC4=O)C3)cn2)cc1Nc1cccc(-c2ncc(F)cn2)c1OC] |
| K149 | 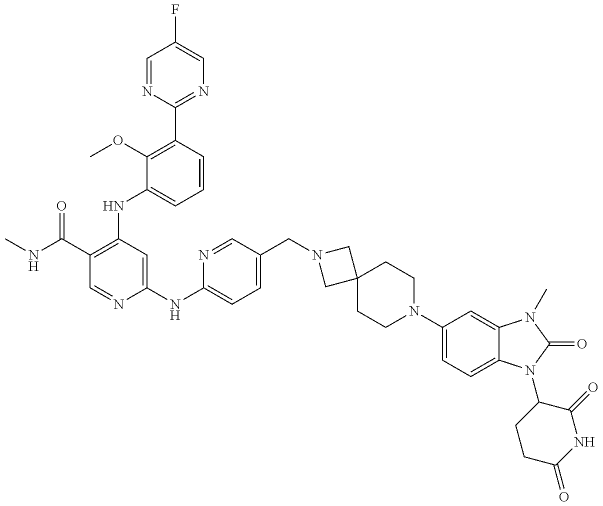 [SMILES: CNC(=O)c1cnc(Nc2ccc(CN3CC4(CCN(c5ccc6c(c5)n(C)c(=O)n6C5CCC(=O)NC5=O)CC4)C3)cn2)cc1Nc1cccc(-c2ncc(F)cn2)c1OC] |
| K150 | 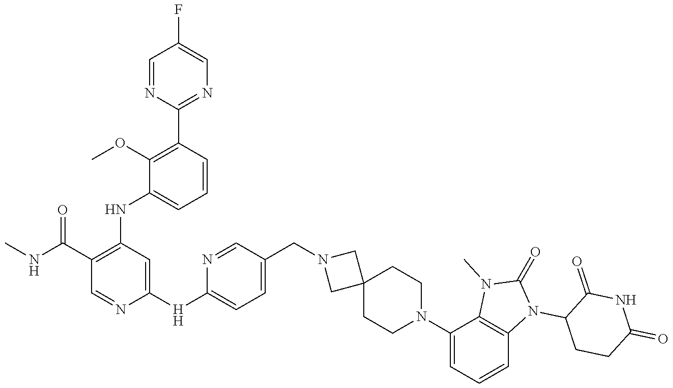 |
| K151 | 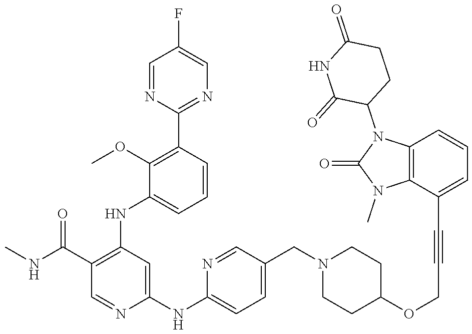 [SMILES: CNC(=O)c1cnc(Nc2ccc(CN3CCC(OCC#Cc4cccc5c4n(C)c(=O)n5C4CCC(=O)NC4=O)CC3)cn2)cc1Nc1cccc(-c2ncc(F)cn2)c1OC] |
| K152 | 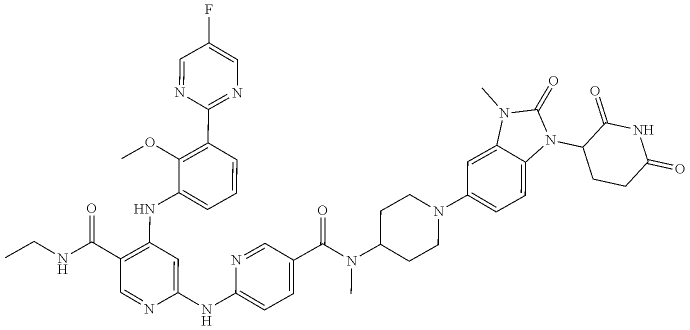 [SMILES: CCNC(=O)c1cnc(Nc2ccc(C(=O)N(C)C3CCN(c4ccc5c(c4)n(C)c(=O)n5C4CCC(=O)NC4=O)CC3)cn2)cc1Nc1cccc(-c2ncc(F)cn2)c1OC] |
| K153 | 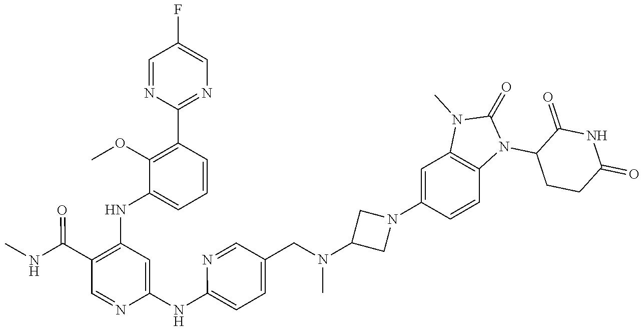 [SMILES: CNC(=O)c1cnc(Nc2ccc(CN(C)C3CN(c4ccc5c(c4)n(C)c(=O)n5C4CCC(=O)NC4=O)C3)cn2)cc1Nc1cccc(-c2ncc(F)cn2)c1OC] |
| K154 | 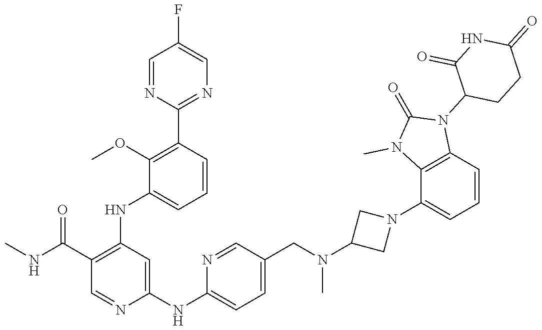 [SMILES: CNC(=O)c1cnc(Nc2ccc(CN(C)C3CN(c4cccc5c4n(C)c(=O)n5C4CCC(=O)NC4=O)C3)cn2)cc1Nc1cccc(-c2ncc(F)cn2)c1OC] |
| K155 | 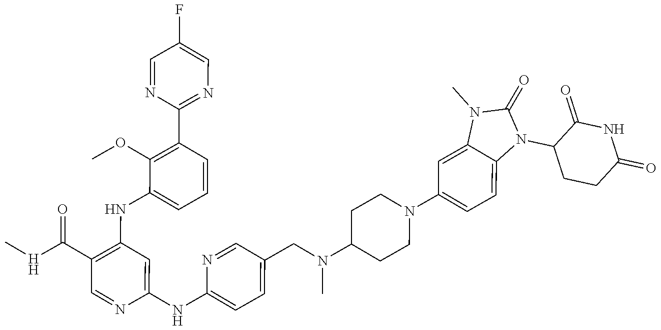 |
| K156 | 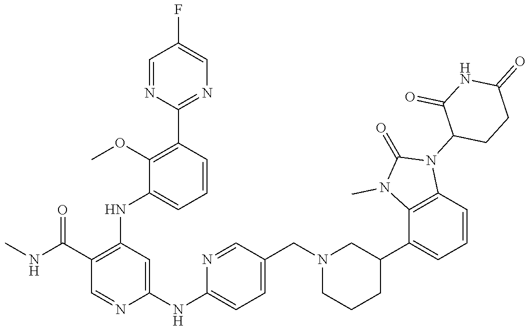 [SMILES: CNC(=O)c1cnc(Nc2ccc(CN3CCCC(c4cccc5c4n(C)c(=O)n5C4CCC(=O)NC4=O)C3)cn2)cc1Nc1cccc(-c2ncc(F)cn2)c1OC] |
| K157 | 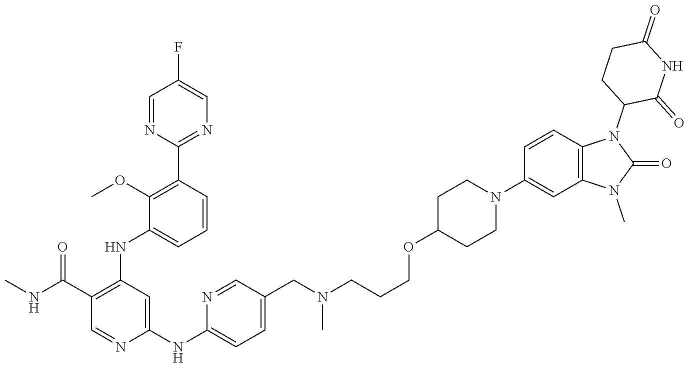 [SMILES: CNC(=O)c1cnc(Nc2ccc(CN(C)CCCOC3CCN(c4ccc5c(c4)n(C)c(=O)n5C4CCC(=O)NC4=O)CC3)cn2)cc1Nc1cccc(-c2ncc(F)cn2)c1OC] |
| K158 | 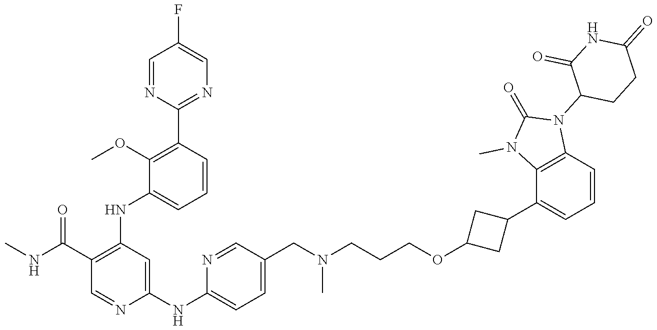 [SMILES: CNC(=O)c1cnc(Nc2ccc(CN(C)CCCOC3CC(c4cccc5c4n(C)c(=O)n5C4CCC(=O)NC4=O)C3)cn2)cc1Nc1cccc(-c2ncc(F)cn2)c1OC] |
| K159 | 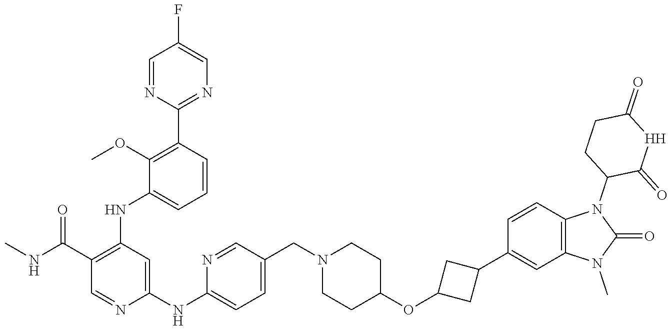 |
| K160 | 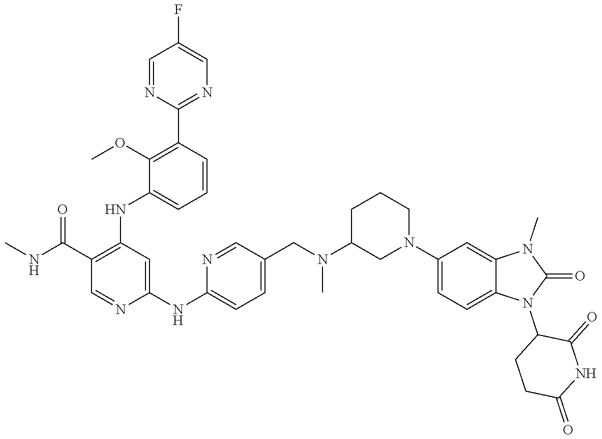 [SMILES: CNC(=O)c1cnc(Nc2ccc(CN(C)C3CCCN(c4ccc5c(c4)n(C)c(=O)n5C4CCC(=O)NC4=O)C3)cn2)cc1Nc1cccc(-c2ncc(F)cn2)c1OC] |
| K161 | 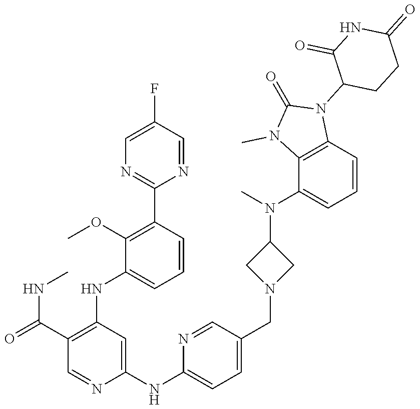 [SMILES: CNC(=O)c1cnc(Nc2ccc(CN3CC(N(C)c4cccc5c4n(C)c(=O)n5C4CCC(=O)NC4=O)C3)cn2)cc1Nc1cccc(-c2ncc(F)cn2)c1OC] |
| K162 | 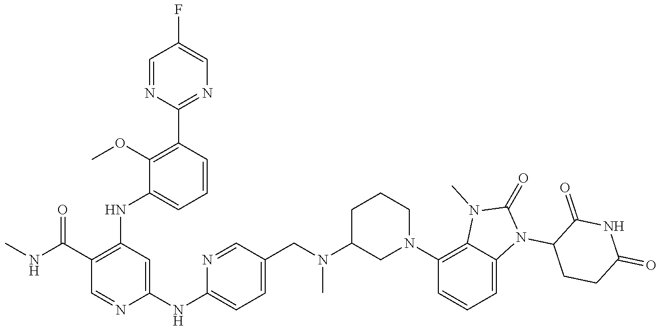 [SMILES: CNC(=O)c1cnc(Nc2ccc(CN(C)C3CCCN(c4cccc5c4n(C)c(=O)n5C4CCC(=O)NC4=O)C3)cn2)cc1Nc1cccc(-c2ncc(F)cn2)c1OC] |
| K163 | 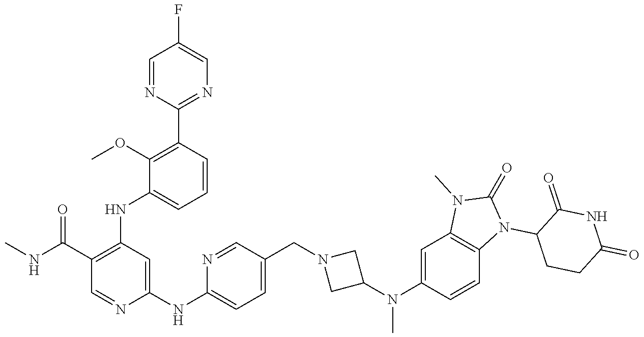 [SMILES: CNC(=O)c1cnc(Nc2ccc(CN3CC(N(C)c4ccc5c(c4)n(C)c(=O)n5C4CCC(=O)NC4=O)C3)cn2)cc1Nc1cccc(-c2ncc(F)cn2)c1OC] |
| K164 | 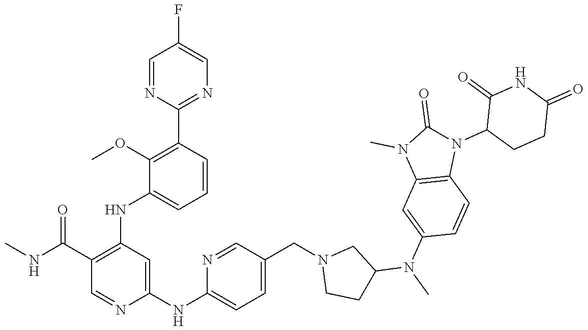 [SMILES: CNC(=O)c1cnc(Nc2ccc(CN3CCC(N(C)c4ccc5c(c4)n(C)c(=O)n5C4CCC(=O)NC4=O)C3)cn2)cc1Nc1cccc(-c2ncc(F)cn2)c1OC] |
| K165 | 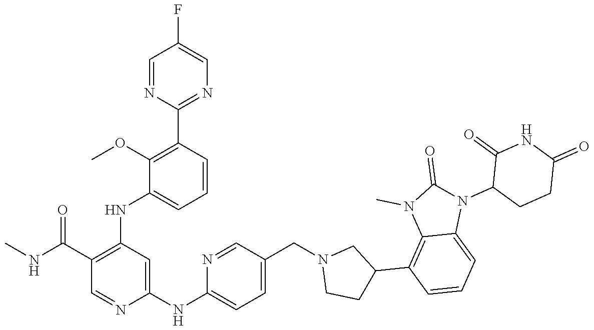 [SMILES: CNC(=O)c1cnc(Nc2ccc(CN3CCC(c4cccc5c4n(C)c(=O)n5C4CCC(=O)NC4=O)C3)cn2)cc1Nc1cccc(-c2ncc(F)cn2)c1OC] |
| K166 | 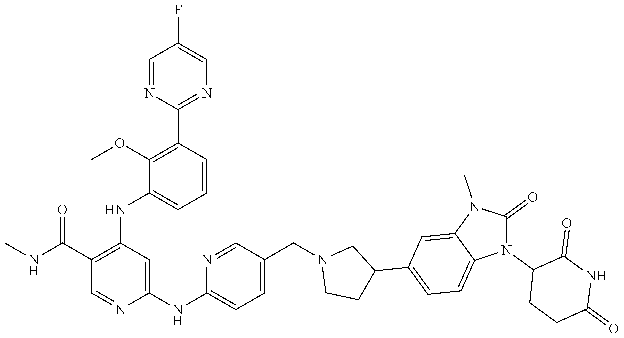 [SMILES: CNC(=O)c1cnc(Nc2ccc(CN3CCC(c4ccc5c(c4)n(C)c(=O)n5C4CCC(=O)NC4=O)C3)cn2)cc1Nc1cccc(-c2ncc(F)cn2)c1OC] |
| K167 | 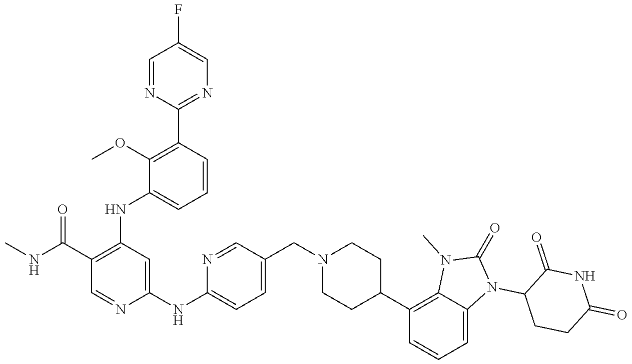 [SMILES: CNC(=O)c1cnc(Nc2ccc(CN3CCC(c4cccc5c4n(C)c(=O)n5C4CCC(=O)NC4=O)CC3)cn2)cc1Nc1cccc(-c2ncc(F)cn2)c1OC] |
| K168 | 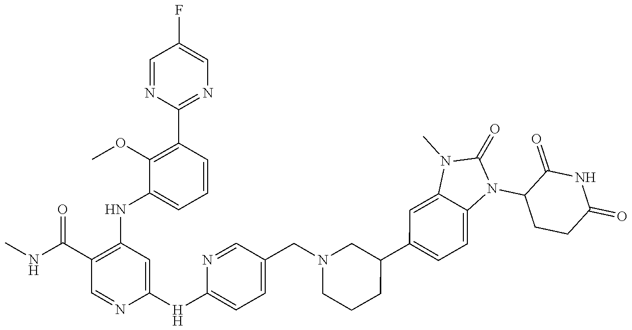 |
| K169 | 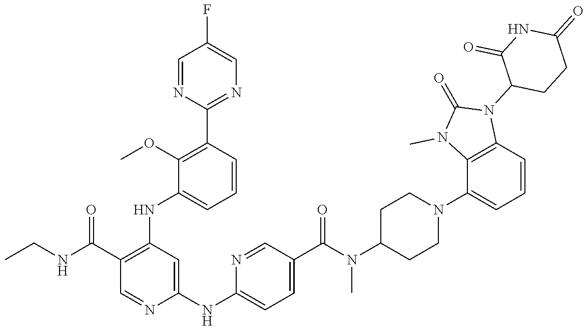 [SMILES: CCNC(=O)c1cnc(Nc2ccc(C(=O)N(C)C3CCN(c4cccc5c4n(C)c(=O)n5C4CCC(=O)NC4=O)CC3)cn2)cc1Nc1cccc(-c2ncc(F)cn2)c1OC] |
| K170 | 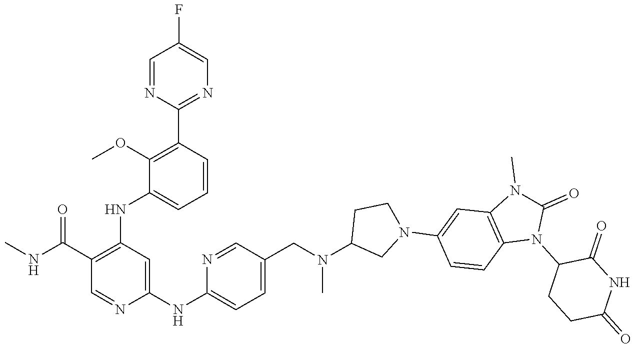 [SMILES: CNC(=O)c1cnc(Nc2ccc(CN(C)C3CCN(c4ccc5c(c4)n(C)c(=O)n5C4CCC(=O)NC4=O)C3)cn2)cc1Nc1cccc(-c2ncc(F)cn2)c1OC] |
| K171 | 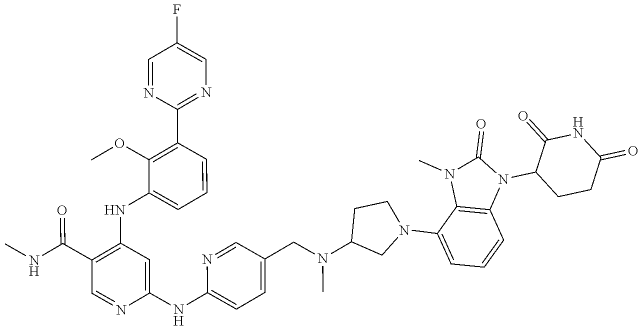 [SMILES: CNC(=O)c1cnc(Nc2ccc(CN(C)C3CCN(c4cccc5c4n(C)c(=O)n5C4CCC(=O)NC4=O)C3)cn2)cc1Nc1cccc(-c2ncc(F)cn2)c1OC] |
| K172 | 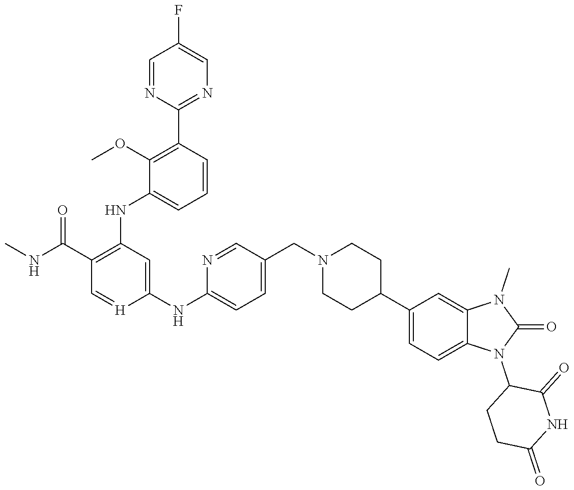 |
| K173 | 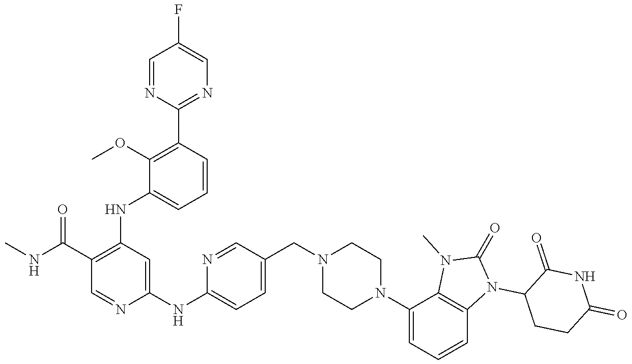 [SMILES: CNC(=O)c1cnc(Nc2ccc(CN3CCN(c4cccc5c4n(C)c(=O)n5C4CCC(=O)NC4=O)CC3)cn2)cc1Nc1cccc(-c2ncc(F)cn2)c1OC] |
| K174 | 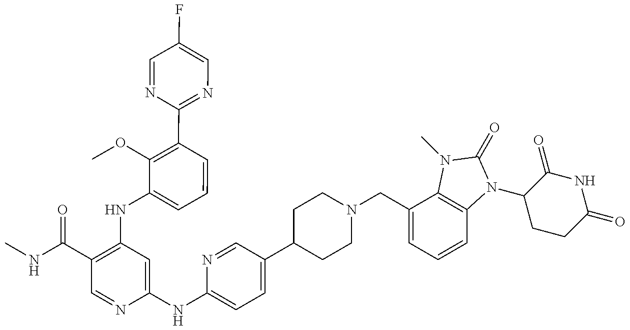 [SMILES: CNC(=O)c1cnc(Nc2ccc(C3CCN(Cc4cccc5c4n(C)c(=O)n5C4CCC(=O)NC4=O)CC3)cn2)cc1Nc1cccc(-c2ncc(F)cn2)c1OC] |
| K175 | 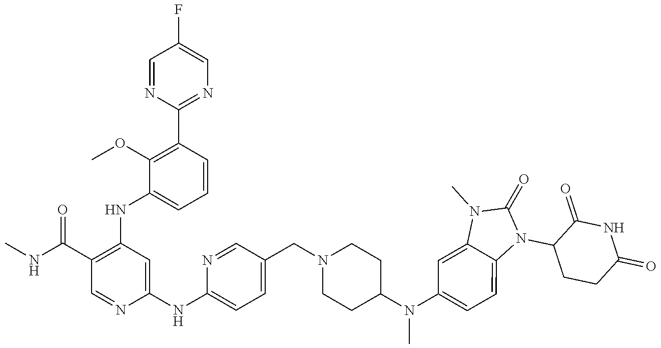 [SMILES: CNC(=O)c1cnc(Nc2ccc(CN3CCC(N(C)c4ccc5c(c4)n(C)c(=O)n5C4CCC(=O)NC4=O)CC3)cn2)cc1Nc1cccc(-c2ncc(F)cn2)c1OC] |
| K176 | 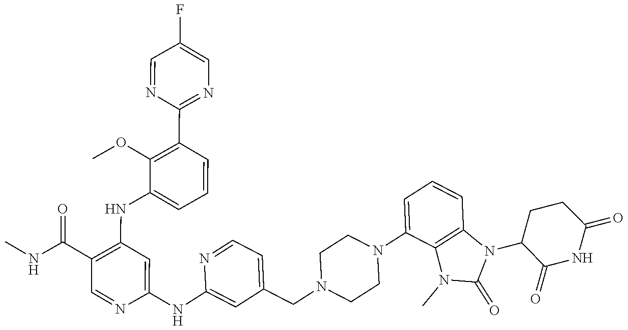 [SMILES: CNC(=O)c1cnc(Nc2cc(CN3CCN(c4cccc5c4n(C)c(=O)n5C4CCC(=O)NC4=O)CC3)ccn2)cc1Nc1cccc(-c2ncc(F)cn2)c1OC] |
| K177 | 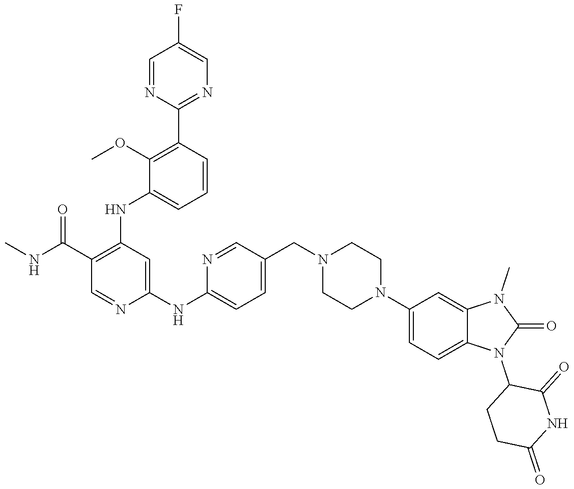 [SMILES: CNC(=O)c1cnc(Nc2ccc(CN3CCN(c4ccc5c(c4)n(C)c(=O)n5C4CCC(=O)NC4=O)CC3)cn2)cc1Nc1cccc(-c2ncc(F)cn2)c1OC] |
| K178 | 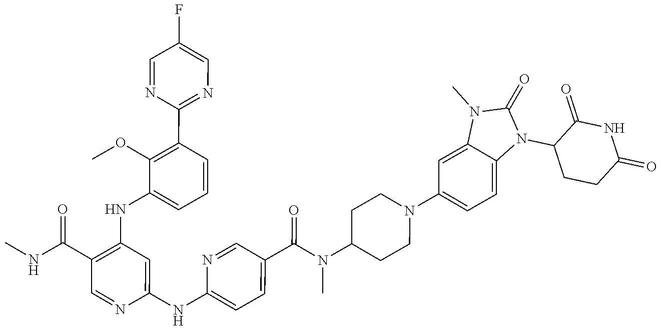 [SMILES: CNC(=O)c1cnc(Nc2ccc(C(=O)N(C)C3CCN(c4ccc5c(c4)n(C)c(=O)n5C4CCC(=O)NC4=O)CC3)cn2)cc1Nc1cccc(-c2ncc(F)cn2)c1OC] |
| K179 | 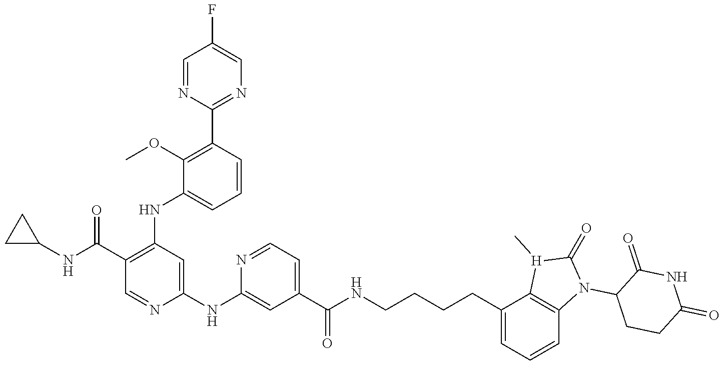 |
| K180 | 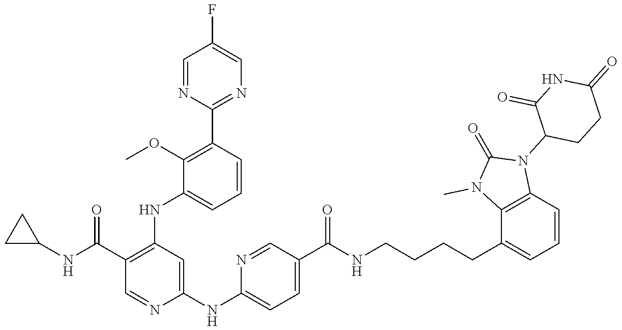 [SMILES: COc1c(Nc2cc(Nc3ccc(C(=O)NCCCCc4cccc5c4n(C)c(=O)n5C4CCC(=O)NC4=O)cn3)ncc2C(=O)NC2CC2)cccc1-c1ncc(F)cn1] |
| K181 | 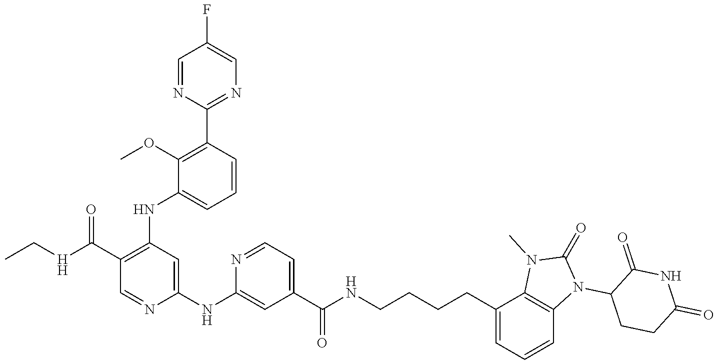 |
| K182 | 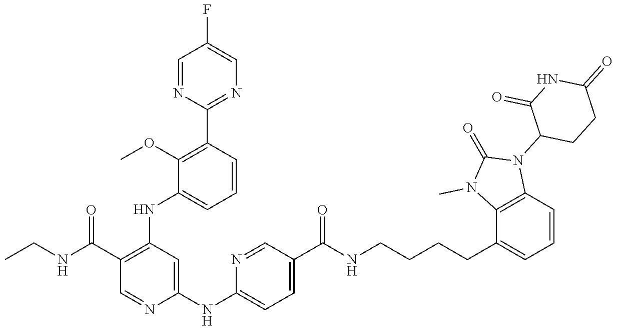 [SMILES: CCNC(=O)c1cnc(Nc2ccc(C(=O)NCCCCc3cccc4c3n(C)c(=O)n4C3CCC(=O)NC3=O)cn2)cc1Nc1cccc(-c2ncc(F)cn2)c1OC] |
| K183 | 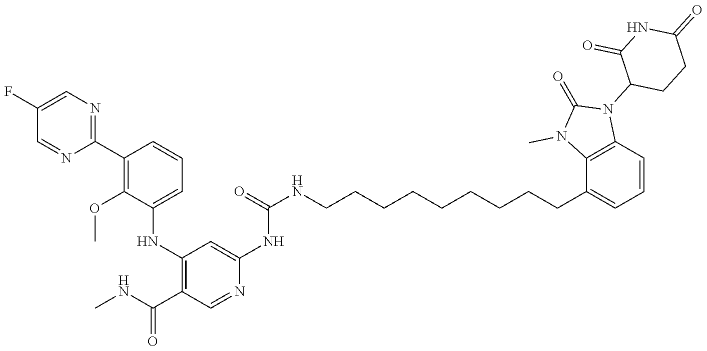 [SMILES: CNC(=O)c1cnc(NC(=O)NCCCCCCCCCc2cccc3c2n(C)c(=O)n3C2CCC(=O)NC2=O)cc1Nc1cccc(-c2ncc(F)cn2)c1OC] |
| K184 | 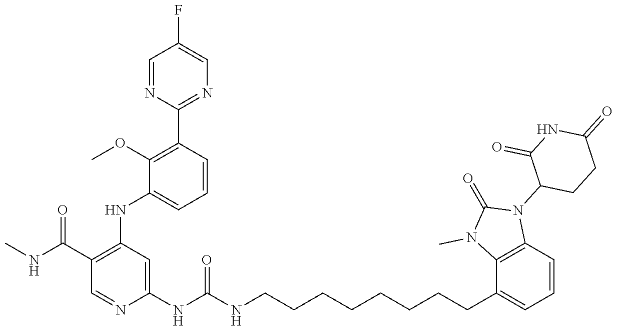 [SMILES: CNC(=O)c1cnc(NC(=O)NCCCCCCCCc2cccc3c2n(C)c(=O)n3C2CCC(=O)NC2=O)cc1Nc1cccc(-c2ncc(F)cn2)c1OC] |
| K185 | 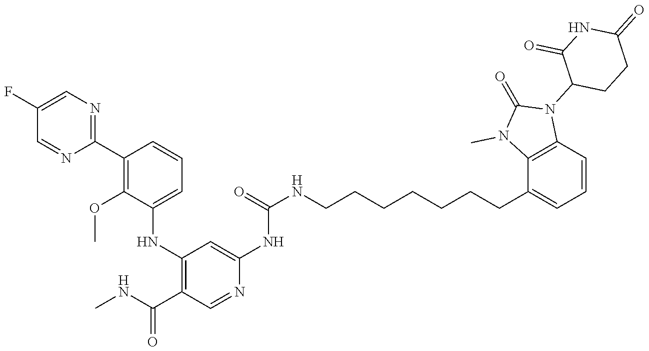 [SMILES: CNC(=O)c1cnc(NC(=O)NCCCCCCCc2cccc3c2n(C)c(=O)n3C2CCC(=O)NC2=O)cc1Nc1cccc(-c2ncc(F)cn2)c1OC] |
| K186 | 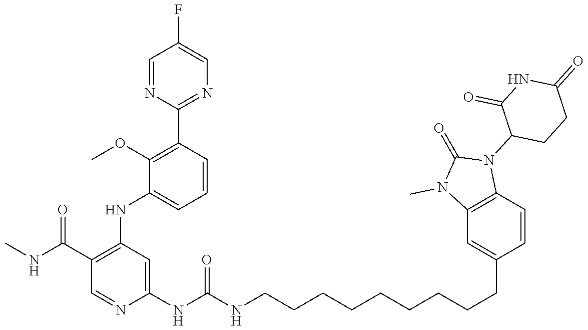 [SMILES: CNC(=O)c1cnc(NC(=O)NCCCCCCCCCc2ccc3c(c2)n(C)c(=O)n3C2CCC(=O)NC2=O)cc1Nc1cccc(-c2ncc(F)cn2)c1OC] |
| K187 | 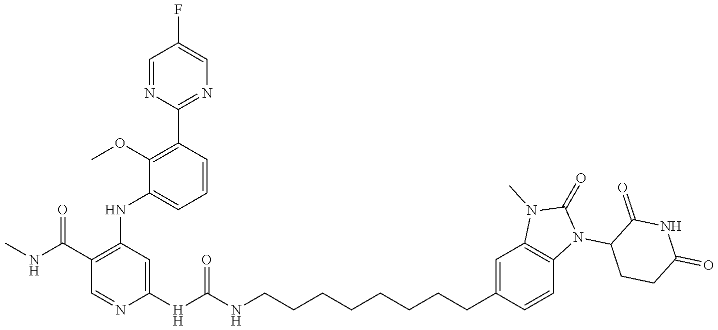 |
| K188 | 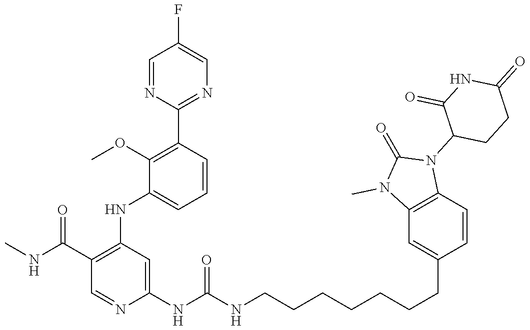 [SMILES: CNC(=O)c1cnc(NC(=O)NCCCCCCCc2ccc3c(c2)n(C)c(=O)n3C2CCC(=O)NC2=O)cc1Nc1cccc(-c2ncc(F)cn2)c1OC] |
| K189 | 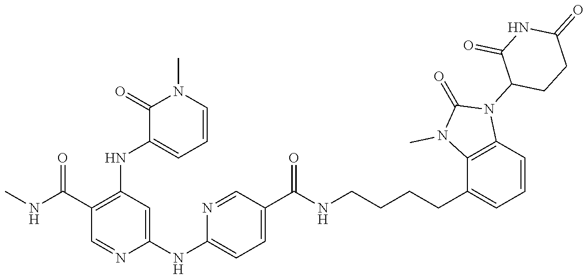 [SMILES: CNC(=O)c1cnc(Nc2ccc(C(=O)NCCCCc3cccc4c3n(C)c(=O)n4C3CCC(=O)NC3=O)cn2)cc1Nc1cccn(C)c1=O] |
| K190 | 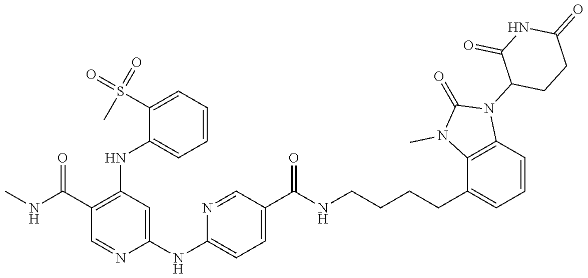 [SMILES: CNC(=O)c1cnc(Nc2ccc(C(=O)NCCCCc3cccc4c3n(C)c(=O)n4C3CCC(=O)NC3=O)cn2)cc1Nc1ccccc1S(C)(=O)=O] |
| K191 | 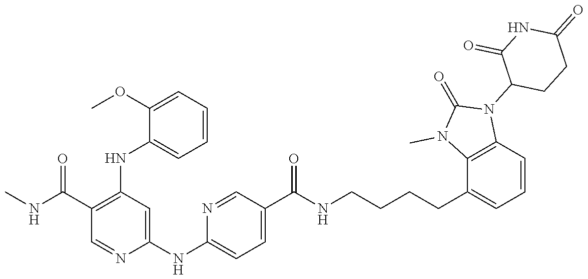 [SMILES: CNC(=O)c1cnc(Nc2ccc(C(=O)NCCCCc3cccc4c3n(C)c(=O)n4C3CCC(=O)NC3=O)cn2)cc1Nc1ccccc1OC] |
| K192 | 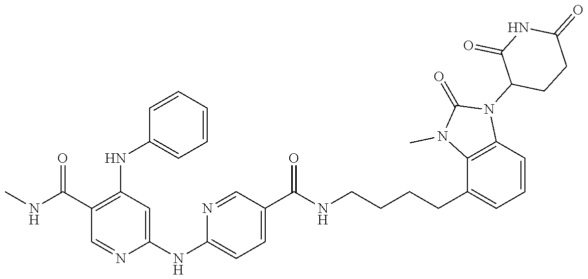 [SMILES: CNC(=O)c1cnc(Nc2ccc(C(=O)NCCCCc3cccc4c3n(C)c(=O)n4C3CCC(=O)NC3=O)cn2)cc1Nc1ccccc1] |
| K193 | 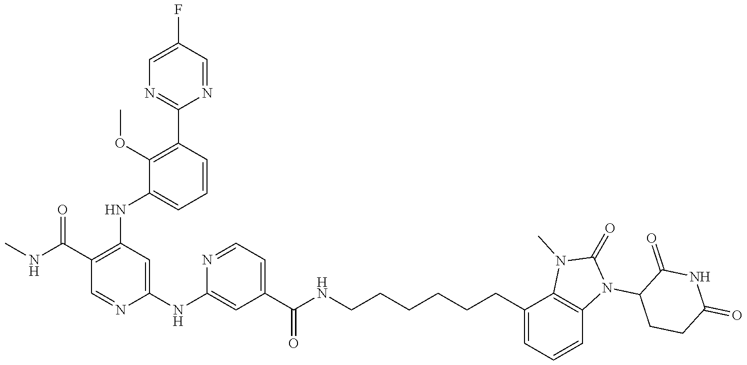 [SMILES: CNC(=O)c1cnc(Nc2cc(C(=O)NCCCCCCc3cccc4c3n(C)c(=O)n4C3CCC(=O)NC3=O)ccn2)cc1Nc1cccc(-c2ncc(F)cn2)c1OC] |
| K194 | 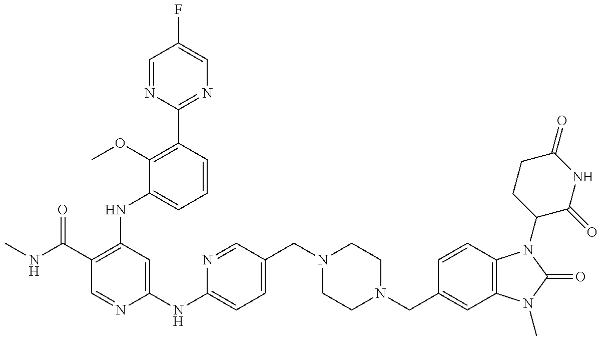 [SMILES: CNC(=O)c1cnc(Nc2ccc(CN3CCN(Cc4ccc5c(c4)n(C)c(=O)n5C4CCC(=O)NC4=O)CC3)cn2)cc1Nc1cccc(-c2ncc(F)cn2)c1OC] |
| K195 | 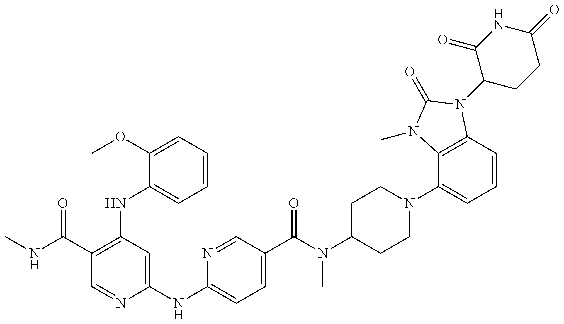 [SMILES: CNC(=O)c1cnc(Nc2ccc(C(=O)N(C)C3CCN(c4cccc5c4n(C)c(=O)n5C4CCC(=O)NC4=O)CC3)cn2)cc1Nc1ccccc1OC] |
| K196 | 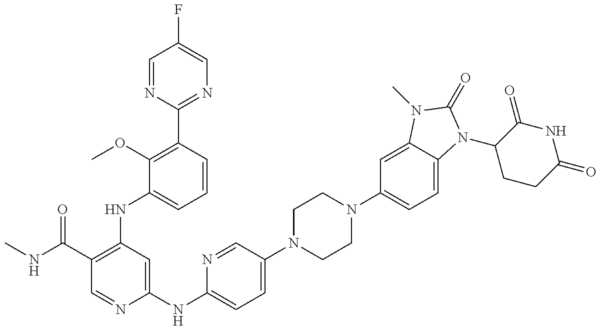 [SMILES: CNC(=O)c1cnc(Nc2ccc(N3CCN(c4ccc5c(c4)n(C)c(=O)n5C4CCC(=O)NC4=O)CC3)cn2)cc1Nc1cccc(-c2ncc(F)cn2)c1OC] |
| K197 | 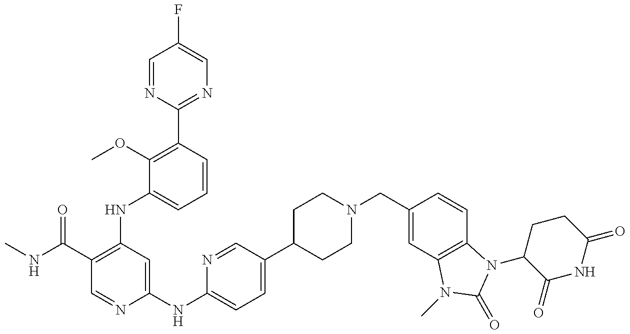 [SMILES: CNC(=O)c1cnc(Nc2ccc(C3CCN(Cc4ccc5c(c4)n(C)c(=O)n5C4CCC(=O)NC4=O)CC3)cn2)cc1Nc1cccc(-c2ncc(F)cn2)c1OC] |
| K198 | 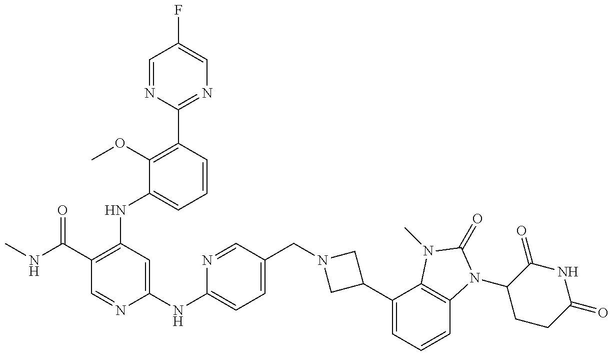 [SMILES: CNC(=O)c1cnc(Nc2ccc(CN3CC(c4cccc5c4n(C)c(=O)n5C4CCC(=O)NC4=O)C3)cn2)cc1Nc1cccc(-c2ncc(F)cn2)c1OC] |
| K199 | 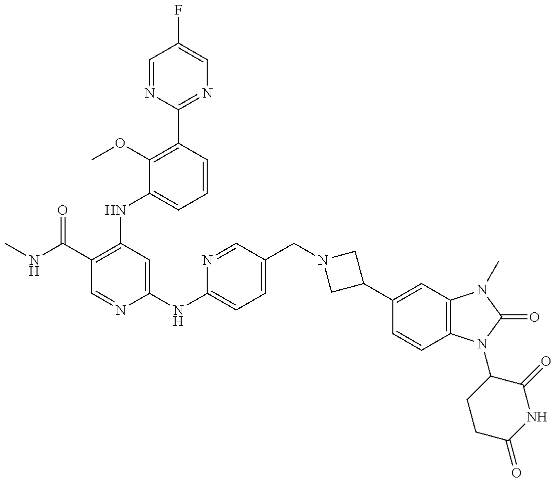 [SMILES: CNC(=O)c1cnc(Nc2ccc(CN3CC(c4ccc5c(c4)n(C)c(=O)n5C4CCC(=O)NC4=O)C3)cn2)cc1Nc1cccc(-c2ncc(F)cn2)c1OC] |
| K700 | 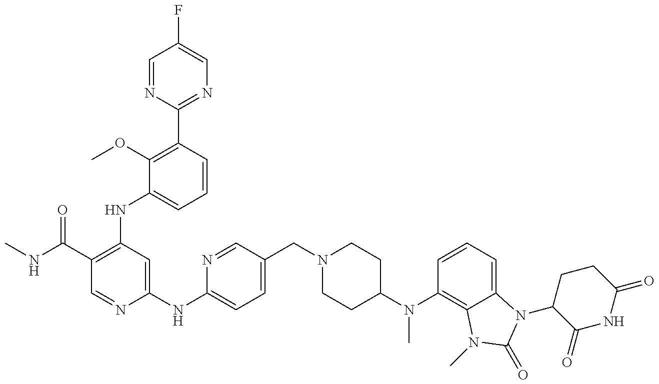 [SMILES: CNC(=O)c1cnc(Nc2ccc(CN3CCC(N(C)c4cccc5c4n(C)c(=O)n5C4CCC(=O)NC4=O)CC3)cn2)cc1Nc1cccc(-c2ncc(F)cn2)c1OC] |
| K701 | 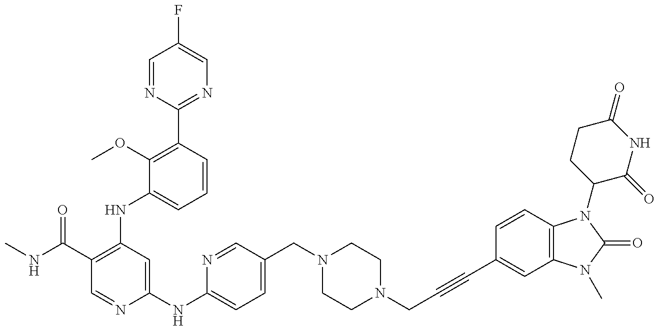 [SMILES: CNC(=O)c1cnc(Nc2ccc(CN3CCN(CC#Cc4ccc5c(c4)n(C)c(=O)n5C4CCC(=O)NC4=O)CC3)cn2)cc1Nc1cccc(-c2ncc(F)cn2)c1OC] |
| K702 | 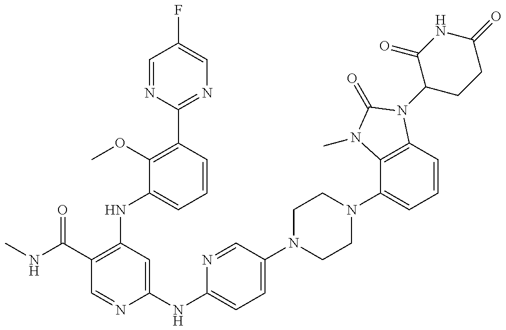 [SMILES: CNC(=O)c1cnc(Nc2ccc(N3CCN(c4cccc5c4n(C)c(=O)n5C4CCC(=O)NC4=O)CC3)cn2)cc1Nc1cccc(-c2ncc(F)cn2)c1OC] |
| K703 | 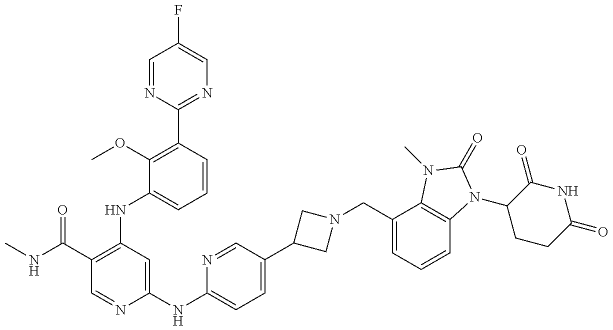 [SMILES: CNC(=O)c1cnc(Nc2ccc(C3CN(Cc4cccc5c4n(C)c(=O)n5C4CCC(=O)NC4=O)C3)cn2)cc1Nc1cccc(-c2ncc(F)cn2)c1OC] |
| K704 | 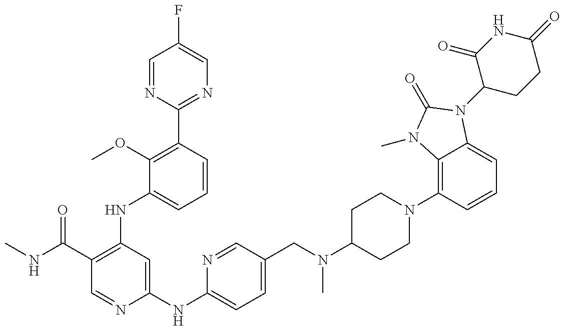 [SMILES: CNC(=O)c1cnc(Nc2ccc(CN(C)C3CCN(c4cccc5c4n(C)c(=O)n5C4CCC(=O)NC4=O)CC3)cn2)cc1Nc1cccc(-c2ncc(F)cn2)c1OC] |
| K705 | 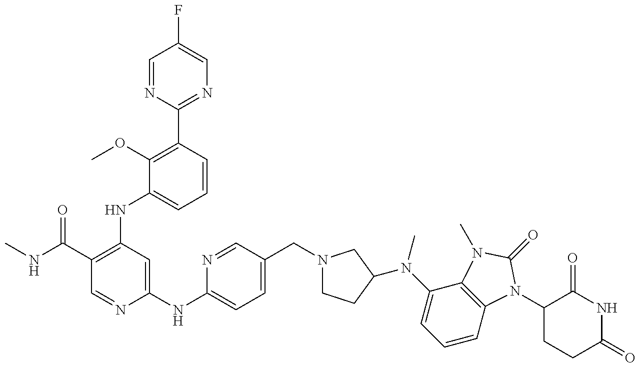 [SMILES: CNC(=O)c1cnc(Nc2ccc(CN3CCC(N(C)c4cccc5c4n(C)c(=O)n5C4CCC(=O)NC4=O)C3)cn2)cc1Nc1cccc(-c2ncc(F)cn2)c1OC] |
| K706 | 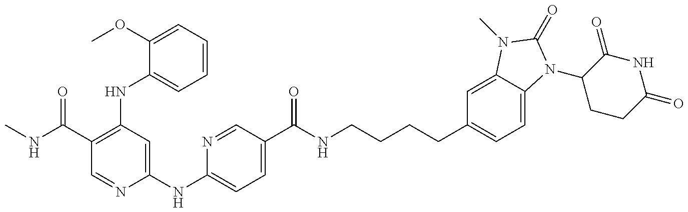 [SMILES: CNC(=O)c1cnc(Nc2ccc(C(=O)NCCCCc3ccc4c(c3)n(C)c(=O)n4C3CCC(=O)NC3=O)cn2)cc1Nc1ccccc1OC] |
| K707 | 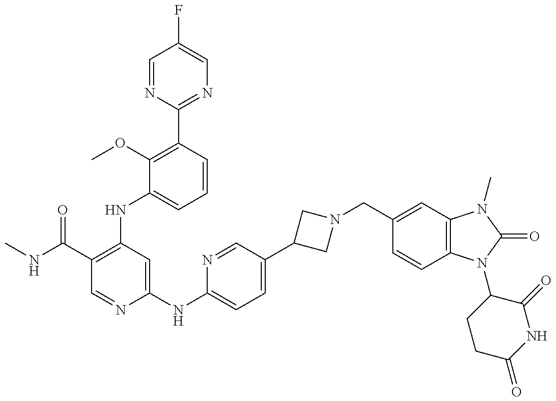 [SMILES: CNC(=O)c1cnc(Nc2ccc(C3CN(Cc4ccc5c(c4)n(C)c(=O)n5C4CCC(=O)NC4=O)C3)cn2)cc1Nc1cccc(-c2ncc(F)cn2)c1OC] |
| K708 | 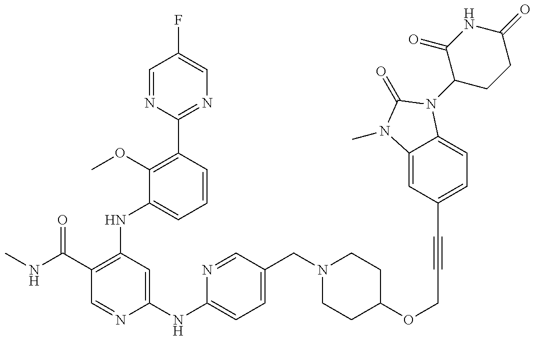 [SMILES: CNC(=O)c1cnc(Nc2ccc(CN3CCC(OCC#Cc4ccc5c(c4)n(C)c(=O)n5C4CCC(=O)NC4=O)CC3)cn2)cc1Nc1cccc(-c2ncc(F)cn2)c1OC] |
| K709 | 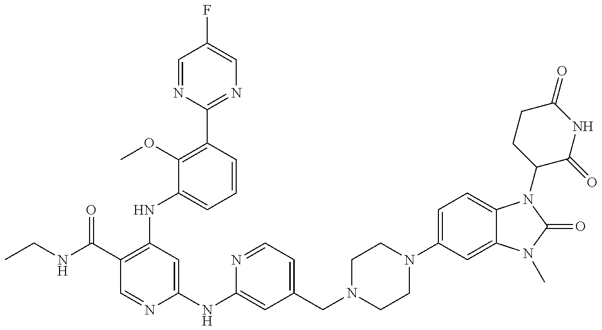 [SMILES: CCNC(=O)c1cnc(Nc2cc(CN3CCN(c4ccc5c(c4)n(C)c(=O)n5C4CCC(=O)NC4=O)CC3)ccn2)cc1Nc1cccc(-c2ncc(F)cn2)c1OC] |
| K710 | 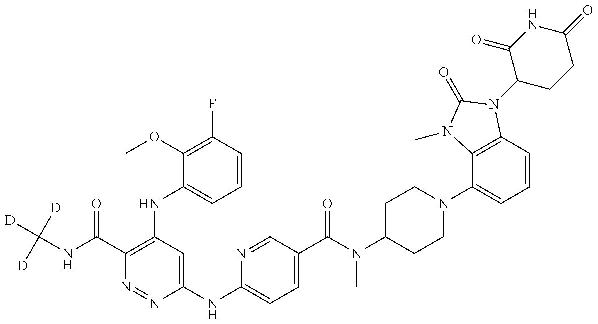 [SMILES: [2H]C([2H])([2H])NC(=O)c1nnc(Nc2ccc(C(=O)N(C)C3CCN(c4cccc5c4n(C)c(=O)n5C4CCC(=O)NC4=O)CC3)cn2)cc1Nc1cccc(F)c1OC] |
| K711 | 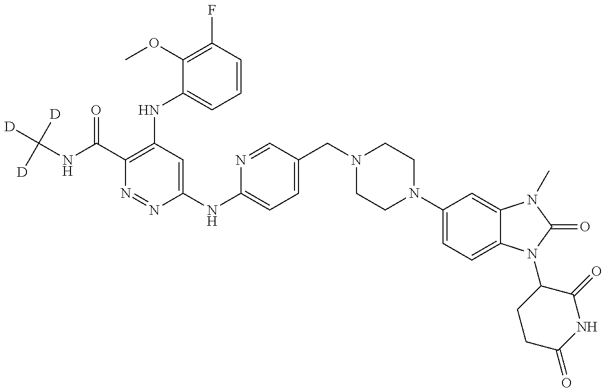 [SMILES: [2H]C([2H])([2H])NC(=O)c1nnc(Nc2ccc(CN3CCN(c4ccc5c(c4)n(C)c(=O)n5C4CCC(=O)NC4=O)CC3)cn2)cc1Nc1cccc(F)c1OC] |
| K712 | 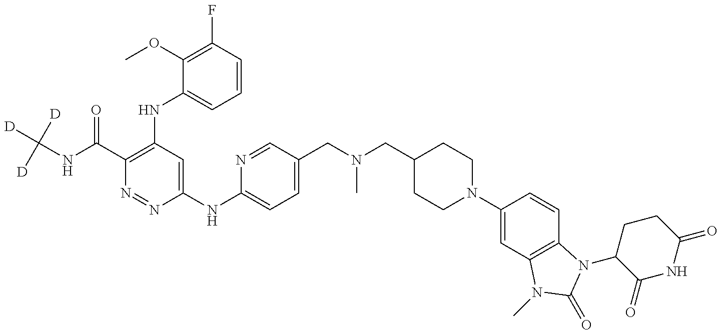 [SMILES: [2H]C([2H])([2H])NC(=O)c1nnc(Nc2ccc(CN(C)CC3CCN(c4ccc5c(c4)n(C)c(=O)n5C4CCC(=O)NC4=O)CC3)cn2)cc1Nc1cccc(F)c1OC] |
| K201 | 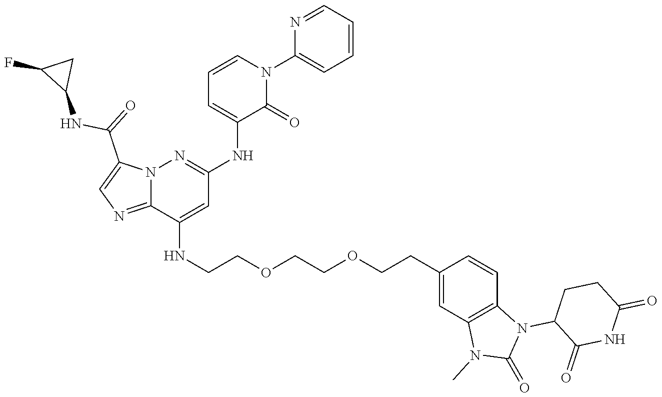 [SMILES: Cn1c(=O)n(C2CCC(=O)NC2=O)c2ccc(CCOCCOCCNc3cc(Nc4cccn(-c5ccccn5)c4=O)nn4c(C(=O)N[C@@H]5C[C@@H]5F)cnc34)cc21] |
| K202 | 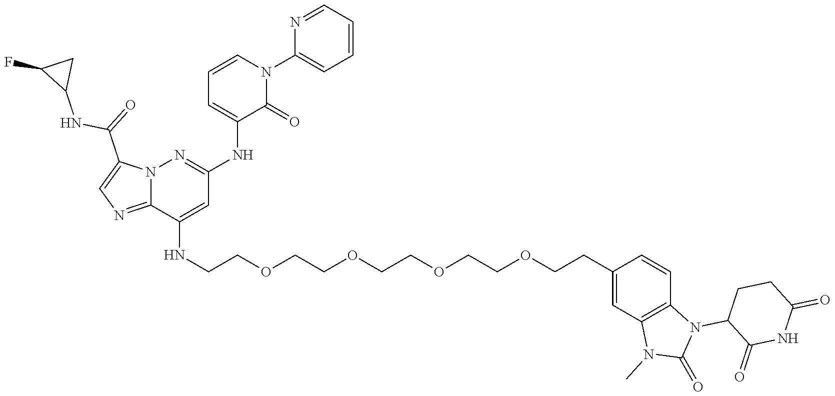 [SMILES: Cn1c(=O)n(C2CCC(=O)NC2=O)c2ccc(CCOCCOCCOCCOCCNc3cc(Nc4cccn(-c5ccccn5)c4=O)nn4c(C(=O)NC5C[C@@H]5F)cnc34)cc21] |
| K203 | 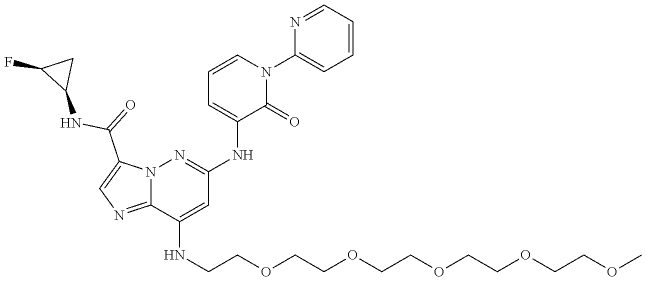 [SMILES: COCCOCCOCCOCCOCCNc1cc(Nc2cccn(-c3ccccn3)c2=O)nn2c(C(=O)N[C@@H]3C[C@@H]3F)cnc12] |
| K204 | 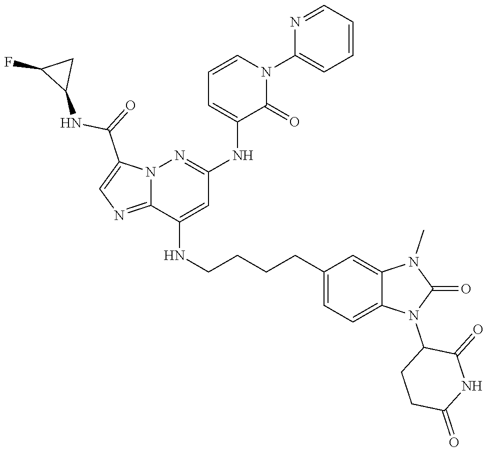 [SMILES: Cn1c(=O)n(C2CCC(=O)NC2=O)c2ccc(CCCCNc3cc(Nc4cccn(-c5ccccn5)c4=O)nn4c(C(=O)N[C@@H]5C[C@@H]5F)cnc34)cc21] |
| K205 | 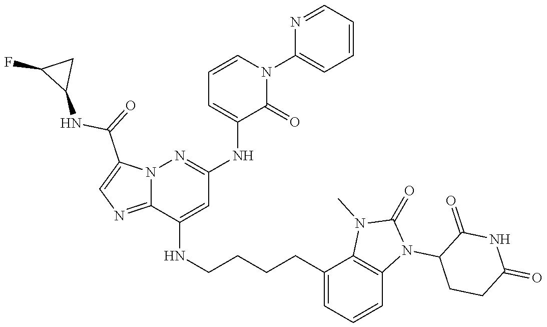 [SMILES: Cn1c(=O)n(C2CCC(=O)NC2=O)c2cccc(CCCCNc3cc(Nc4cccn(-c5ccccn5)c4=O)nn4c(C(=O)N[C@@H]5C[C@@H]5F)cnc34)c21] |
| K206 | 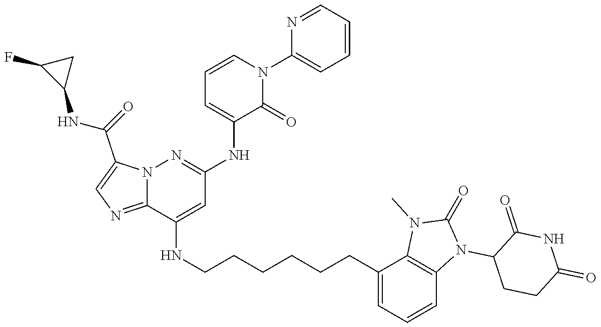 [SMILES: Cn1c(=O)n(C2CCC(=O)NC2=O)c2cccc(CCCCCCNc3cc(Nc4cccn(-c5ccccn5)c4=O)nn4c(C(=O)N[C@@H]5C[C@@H]5F)cnc34)c21] |
| K207 | 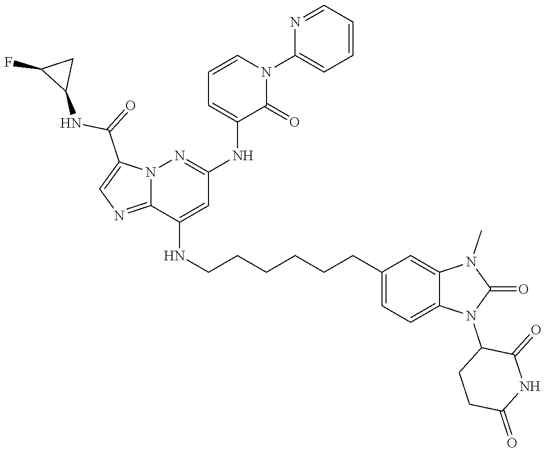 [SMILES: Cn1c(=O)n(C2CCC(=O)NC2=O)c2ccc(CCCCCCNc3cc(Nc4cccn(-c5ccccn5)c4=O)nn4c(C(=O)N[C@@H]5C[C@@H]5F)cnc34)cc21] |
| K208 | 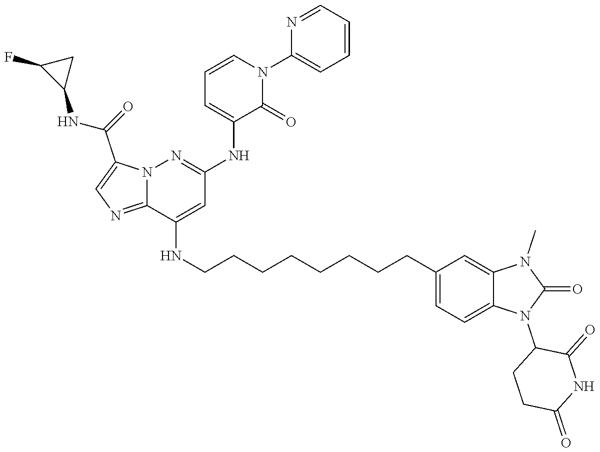 [SMILES: Cn1c(=O)n(C2CCC(=O)NC2=O)c2ccc(CCCCCCCCNc3cc(Nc4cccn(-c5ccccn5)c4=O)nn4c(C(=O)N[C@@H]5C[C@@H]5F)cnc34)cc21] |
| K209 | 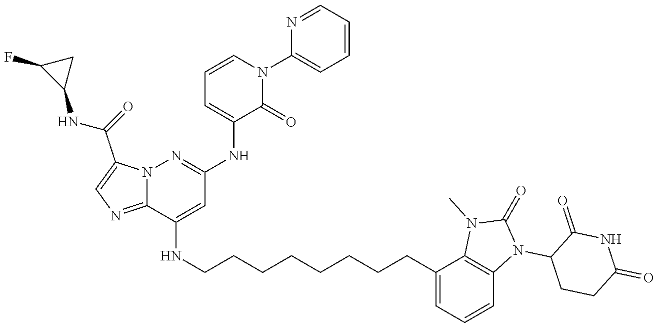 [SMILES: Cn1c(=O)n(C2CCC(=O)NC2=O)c2cccc(CCCCCCCCNc3cc(Nc4cccn(-c5ccccn5)c4=O)nn4c(C(=O)N[C@@H]5C[C@@H]5F)cnc34)c21] |
| K210 | 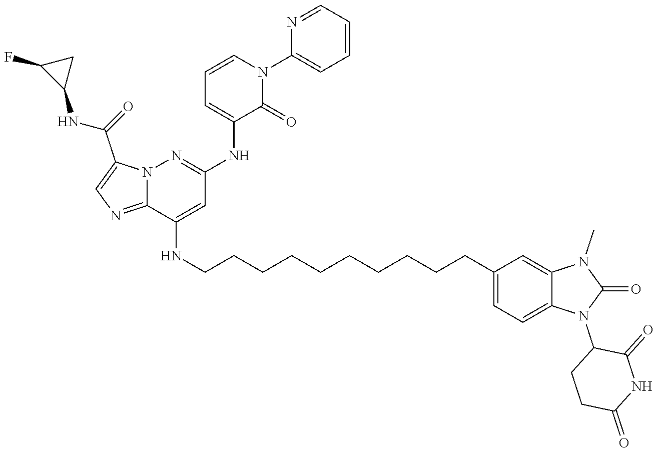 [SMILES: Cn1c(=O)n(C2CCC(=O)NC2=O)c2ccc(CCCCCCCCCCNc3cc(Nc4cccn(-c5ccccn5)c4=O)nn4c(C(=O)N[C@@H]5C[C@@H]5F)cnc34)cc21] |
| K211 | 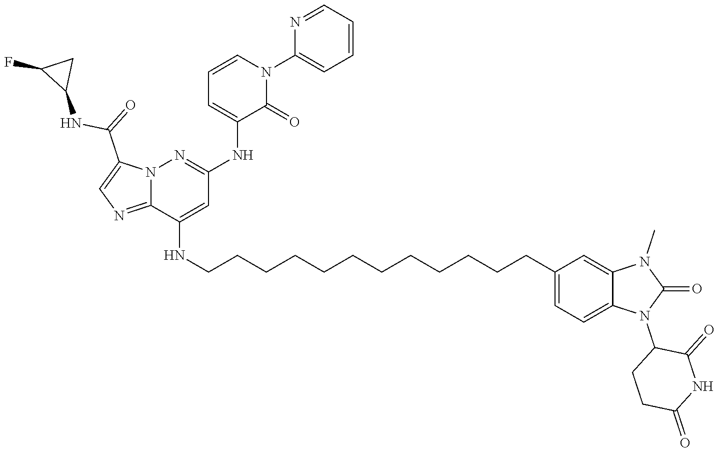 [SMILES: Cn1c(=O)n(C2CCC(=O)NC2=O)c2ccc(CCCCCCCCCCCCNc3cc(Nc4cccn(-c5ccccn5)c4=O)nn4c(C(=O)N[C@@H]5C[C@@H]5F)cnc34)cc21] |
| K212 | 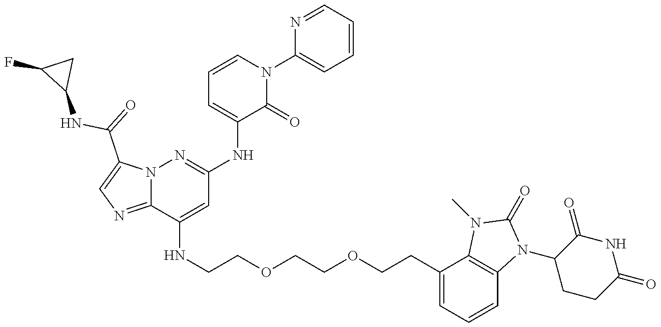 [SMILES: Cn1c(=O)n(C2CCC(=O)NC2=O)c2cccc(CCOCCOCCNc3cc(Nc4cccn(-c5ccccn5)c4=O)nn4c(C(=O)N[C@@H]5C[C@@H]5F)cnc34)c21] |
| K213 | 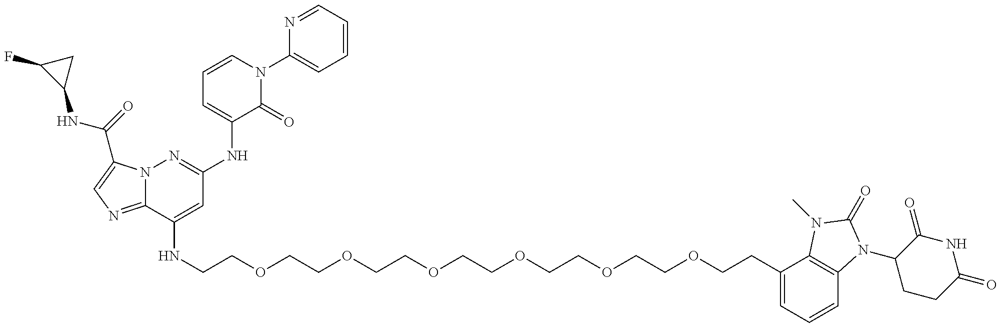 [SMILES: Cn1c(=O)n(C2CCC(=O)NC2=O)c2cccc(CCOCCOCCOCCOCCOCCOCCNc3cc(Nc4cccn(-c5ccccn5)c4=O)nn4c(C(=O)N[C@@H]5C[C@@H]5F)cnc34)c21] |
| K214 | 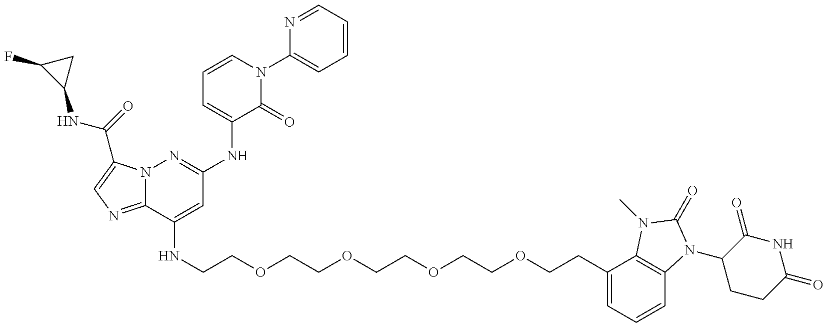 [SMILES: Cn1c(=O)n(C2CCC(=O)NC2=O)c2cccc(CCOCCOCCOCCOCCNc3cc(Nc4cccn(-c5ccccn5)c4=O)nn4c(C(=O)N[C@@H]5C[C@@H]5F)cnc34)c21] |
| K215 | 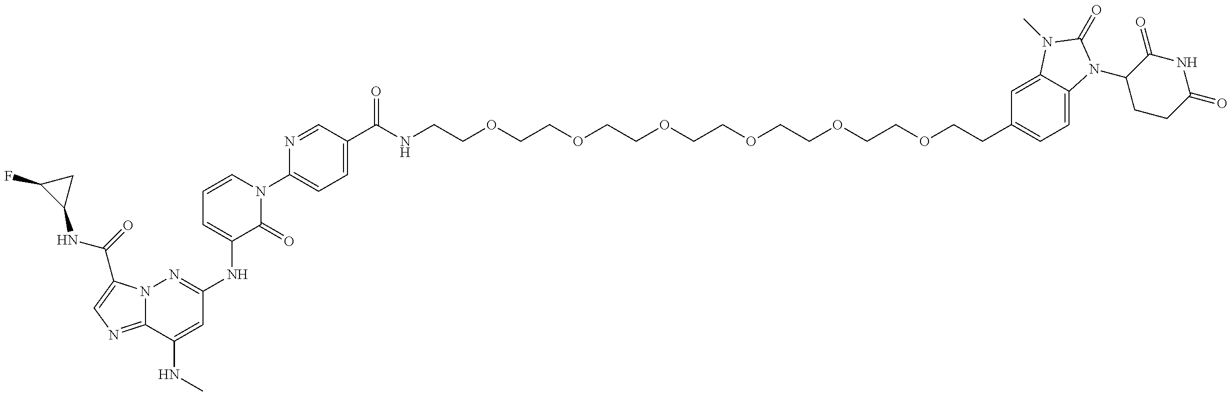 [SMILES: CNc1cc(Nc2cccn(-c3ccc(C(=O)NCCOCCOCCOCCOCCOCCOCCc4ccc5c(c4)n(C)c(=O)n5C4CCC(=O)NC4=O)cn3)c2=O)nn2c(C(=O)N[C@@H]3C[C@@H]3F)cnc12] |
| K216 | 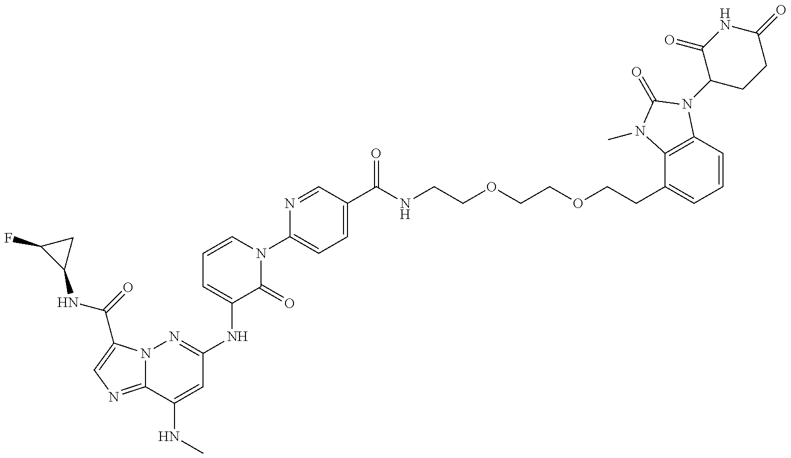 [SMILES: CNc1cc(Nc2cccn(-c3ccc(C(=O)NCCOCCOCCc4cccc5c4n(C)c(=O)n5C4CCC(=O)NC4=O)cn3)c2=O)nn2c(C(=O)N[C@@H]3C[C@@H]3F)cnc12] |
| K217 | 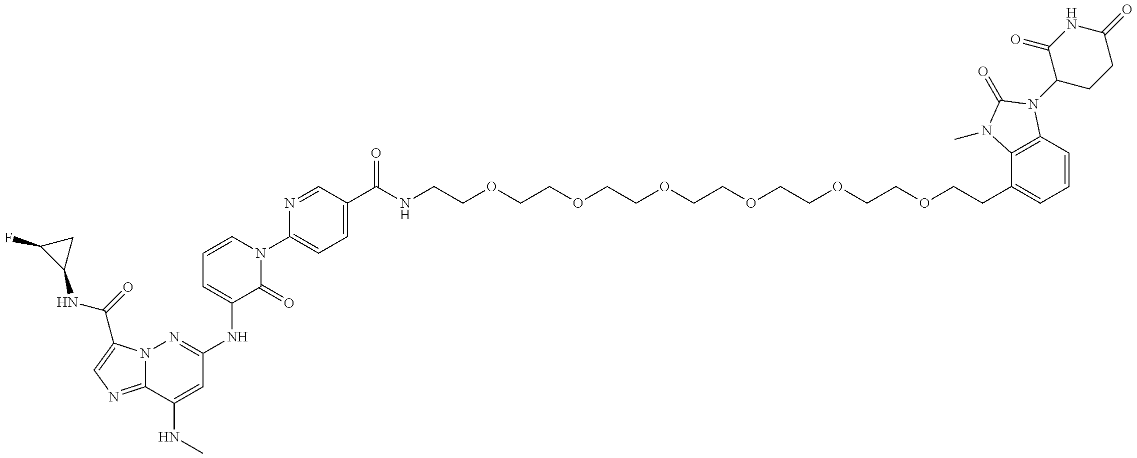 [SMILES: CNc1cc(Nc2cccn(-c3ccc(C(=O)NCCOCCOCCOCCOCCOCCOCCc4cccc5c4n(C)c(=O)n5C4CCC(=O)NC4=O)cn3)c2=O)nn2c(C(=O)N[C@@H]3C[C@@H]3F)cnc12] |
| K218 | 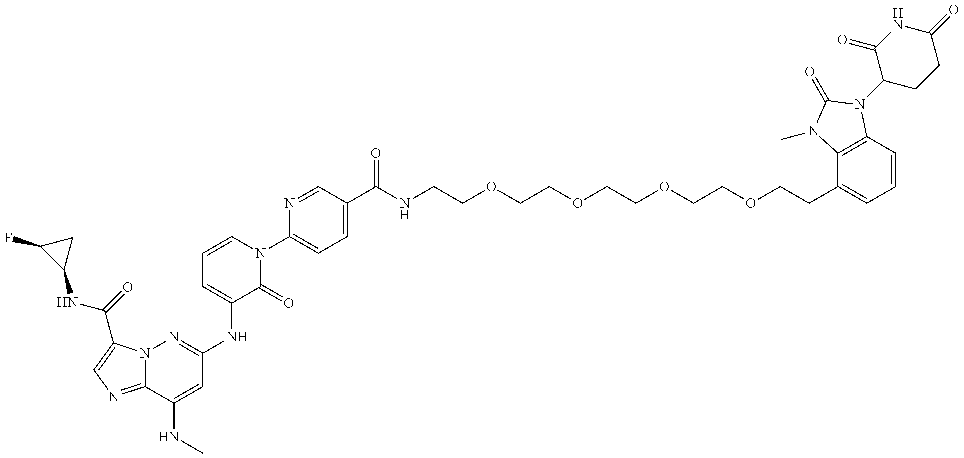 [SMILES: CNc1cc(Nc2cccn(-c3ccc(C(=O)NCCOCCOCCOCCOCCc4cccc5c4n(C)c(=O)n5C4CCC(=O)NC4=O)cn3)c2=O)nn2c(C(=O)N[C@@H]3C[C@@H]3F)cnc12] |
| K219 | 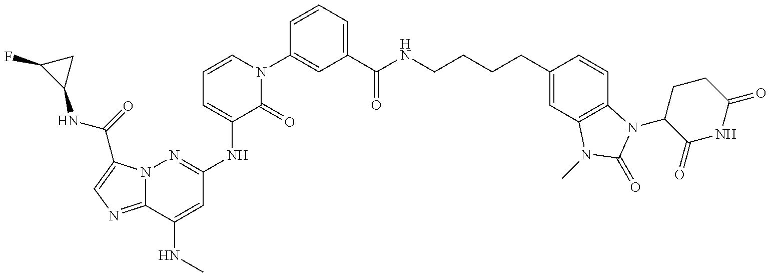 [SMILES: CNc1cc(Nc2cccn(-c3cccc(C(=O)NCCCCc4ccc5c(c4)n(C)c(=O)n5C4CCC(=O)NC4=O)c3)c2=O)nn2c(C(=O)N[C@@H]3C[C@@H]3F)cnc12] |
| K220 | 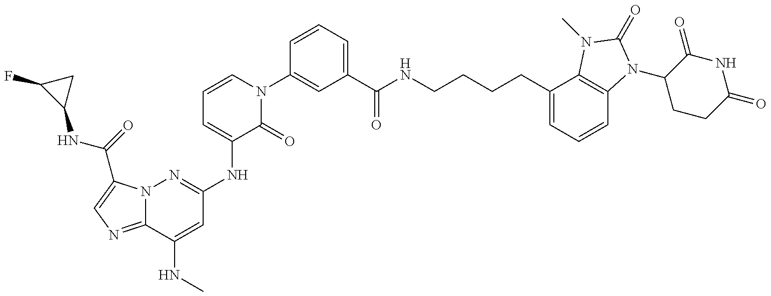 [SMILES: CNc1cc(Nc2cccn(-c3cccc(C(=O)NCCCCc4cccc5c4n(C)c(=O)n5C4CCC(=O)NC4=O)c3)c2=O)nn2c(C(=O)N[C@@H]3C[C@@H]3F)cnc12] |
| K221 | 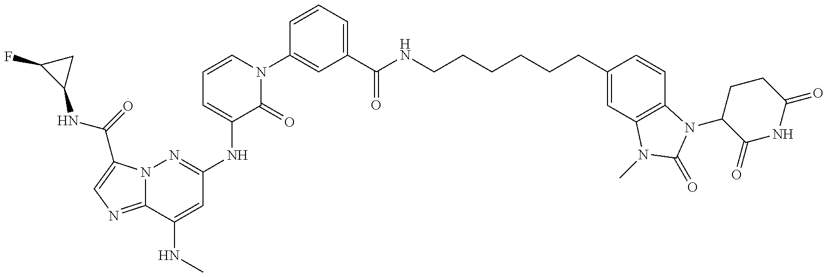 [SMILES: CNc1cc(Nc2cccn(-c3cccc(C(=O)NCCCCCCc4ccc5c(c4)n(C)c(=O)n5C4CCC(=O)NC4=O)c3)c2=O)nn2c(C(=O)N[C@@H]3C[C@@H]3F)cnc12] |
| K222 | 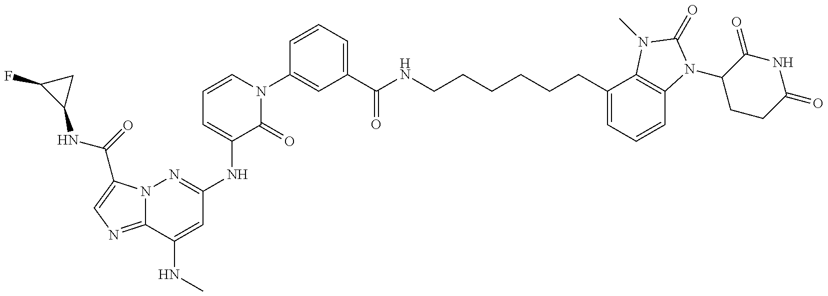 [SMILES: CNc1cc(Nc2cccn(-c3cccc(C(=O)NCCCCCCc4cccc5c4n(C)c(=O)n5C4CCC(=O)NC4=O)c3)c2=O)nn2c(C(=O)N[C@@H]3C[C@@H]3F)cnc12] |
| K223 | 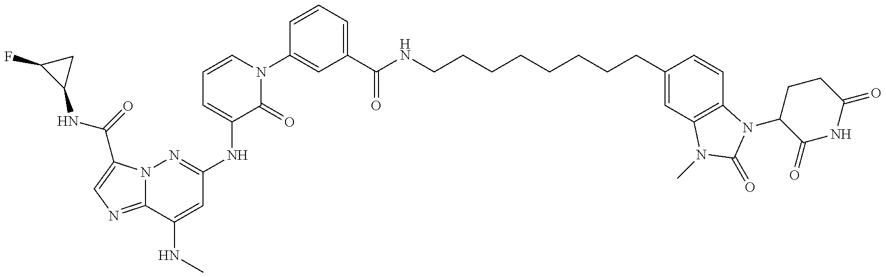 [SMILES: CNc1cc(Nc2cccn(-c3cccc(C(=O)NCCCCCCCCc4ccc5c(c4)n(C)c(=O)n5C4CCC(=O)NC4=O)c3)c2=O)nn2c(C(=O)N[C@@H]3C[C@@H]3F)cnc12] |
| K224 | 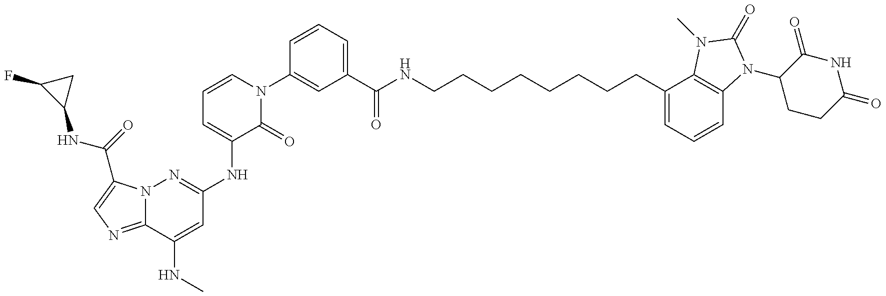 [SMILES: CNc1cc(Nc2cccn(-c3cccc(C(=O)NCCCCCCCCc4cccc5c4n(C)c(=O)n5C4CCC(=O)NC4=O)c3)c2=O)nn2c(C(=O)N[C@@H]3C[C@@H]3F)cnc12] |
| K225 | 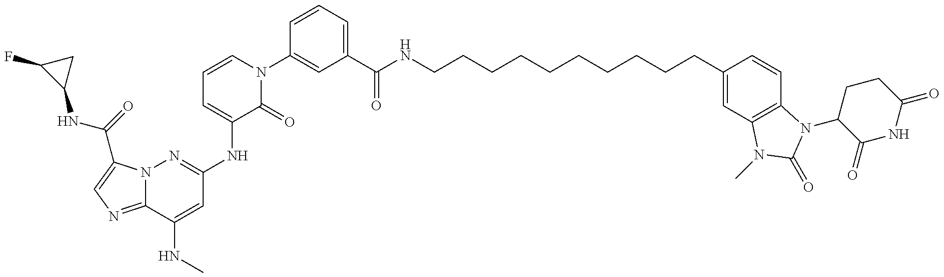 [SMILES: CNc1cc(Nc2cccn(-c3cccc(C(=O)NCCCCCCCCCCc4ccc5c(c4)n(C)c(=O)n5C4CCC(=O)NC4=O)c3)c2=O)nn2c(C(=O)N[C@@H]3C[C@@H]3F)cnc12] |
| K226 | 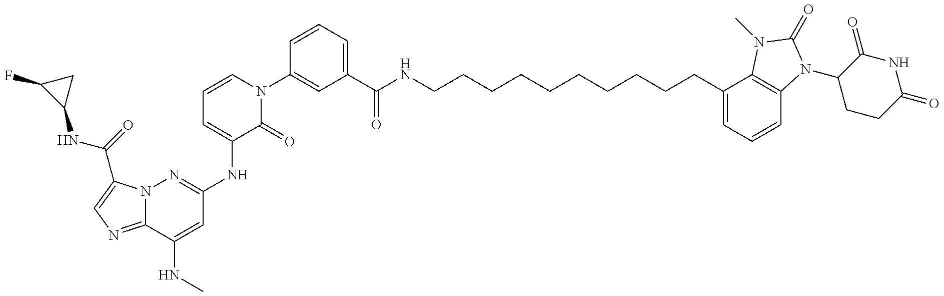 [SMILES: CNc1cc(Nc2cccn(-c3cccc(C(=O)NCCCCCCCCCCc4cccc5c4n(C)c(=O)n5C4CCC(=O)NC4=O)c3)c2=O)nn2c(C(=O)N[C@@H]3C[C@@H]3F)cnc12] |
| K227 | 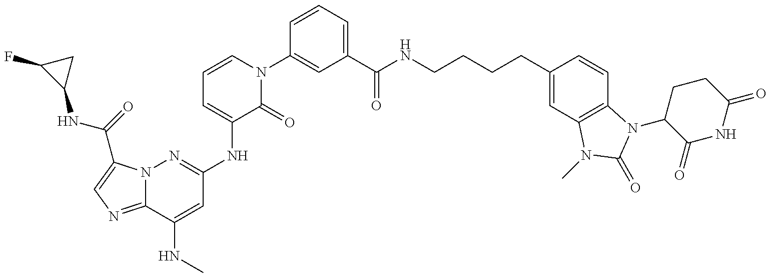 [SMILES: CNc1cc(Nc2cccn(-c3cccc(C(=O)NCCCCc4ccc5c(c4)n(C)c(=O)n5C4CCC(=O)NC4=O)c3)c2=O)nn2c(C(=O)N[C@@H]3C[C@@H]3F)cnc12] |
| K228 | 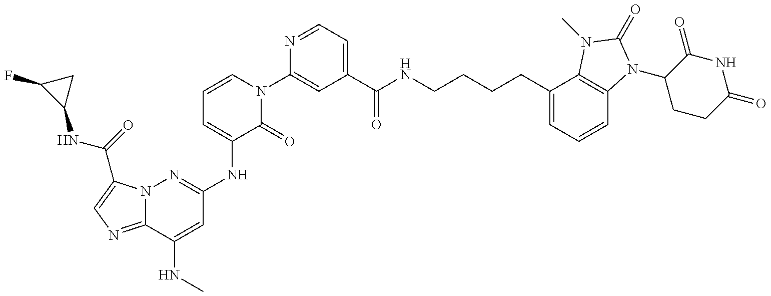 [SMILES: CNc1cc(Nc2cccn(-c3cc(C(=O)NCCCCc4cccc5c4n(C)c(=O)n5C4CCC(=O)NC4=O)ccn3)c2=O)nn2c(C(=O)N[C@@H]3C[C@@H]3F)cnc12] |
| K229 | 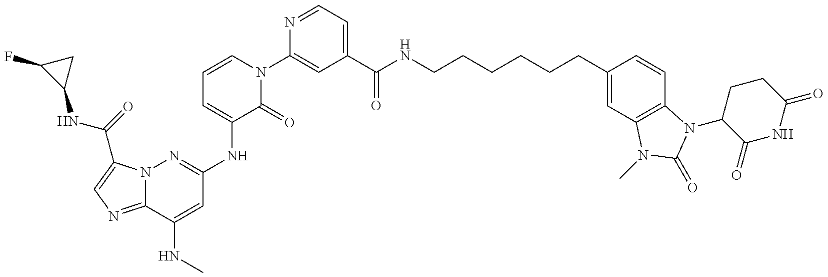 [SMILES: CNc1cc(Nc2cccn(-c3cc(C(=O)NCCCCCCc4ccc5c(c4)n(C)c(=O)n5C4CCC(=O)NC4=O)ccn3)c2=O)nn2c(C(=O)N[C@@H]3C[C@@H]3F)cnc12] |
| K230 | 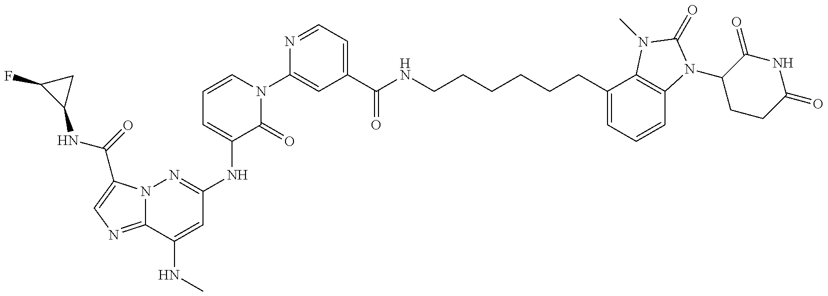 [SMILES: CNc1cc(Nc2cccn(-c3cc(C(=O)NCCCCCCc4cccc5c4n(C)c(=O)n5C4CCC(=O)NC4=O)ccn3)c2=O)nn2c(C(=O)N[C@@H]3C[C@@H]3F)cnc12] |
| K231 | 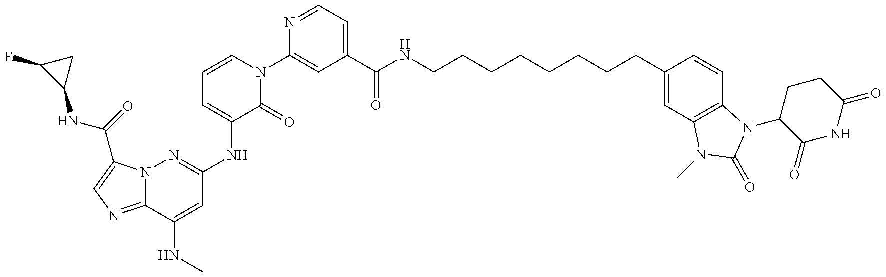 [SMILES: CNc1cc(Nc2cccn(-c3cc(C(=O)NCCCCCCCCc4ccc5c(c4)n(C)c(=O)n5C4CCC(=O)NC4=O)ccn3)c2=O)nn2c(C(=O)N[C@@H]3C[C@@H]3F)cnc12] |
| K232 | 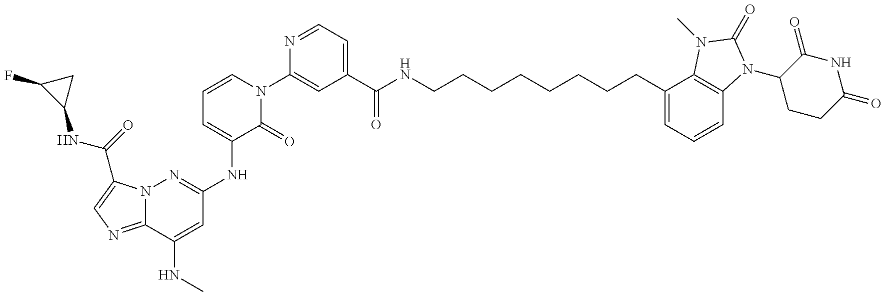 [SMILES: CNc1cc(Nc2cccn(-c3cc(C(=O)NCCCCCCCCc4cccc5c4n(C)c(=O)n5C4CCC(=O)NC4=O)ccn3)c2=O)nn2c(C(=O)N[C@@H]3C[C@@H]3F)cnc12] |
| K233 | 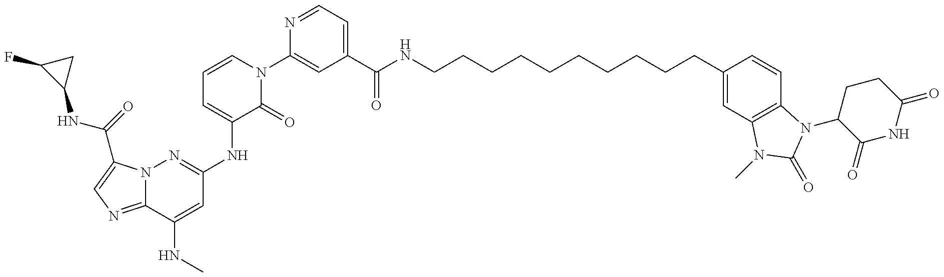 [SMILES: CNc1cc(Nc2cccn(-c3cc(C(=O)NCCCCCCCCCCc4ccc5c(c4)n(C)c(=O)n5C4CCC(=O)NC4=O)ccn3)c2=O)nn2c(C(=O)N[C@@H]3C[C@@H]3F)cnc12] |
| K234 | 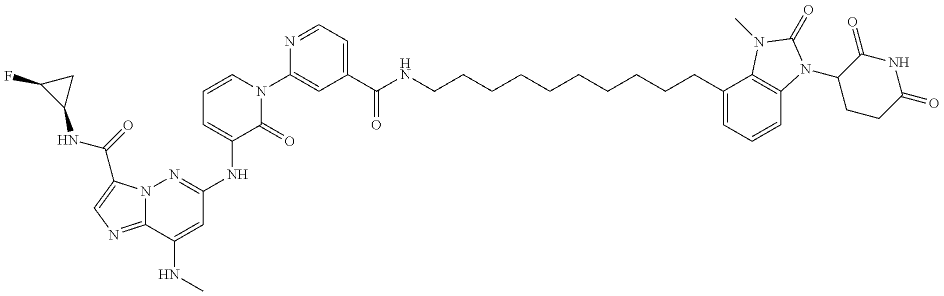 [SMILES: CNc1cc(Nc2cccn(-c3cc(C(=O)NCCCCCCCCCCc4cccc5c4n(C)c(=O)n5C4CCC(=O)NC4=O)ccn3)c2=O)nn2c(C(=O)N[C@@H]3C[C@@H]3F)cnc12] |
| K235 | 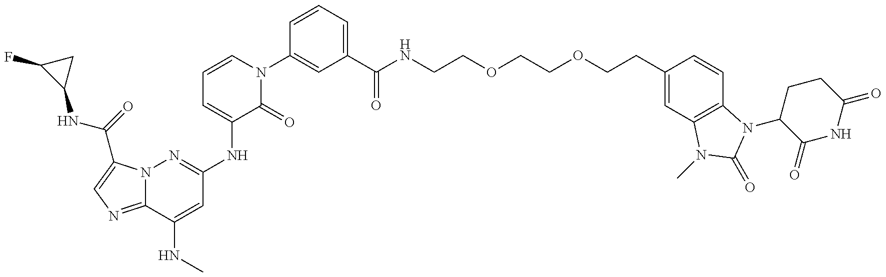 [SMILES: CNc1cc(Nc2cccn(-c3cccc(C(=O)NCCOCCOCCc4ccc5c(c4)n(C)c(=O)n5C4CCC(=O)NC4=O)c3)c2=O)nn2c(C(=O)N[C@@H]3C[C@@H]3F)cnc12] |
| K236 | 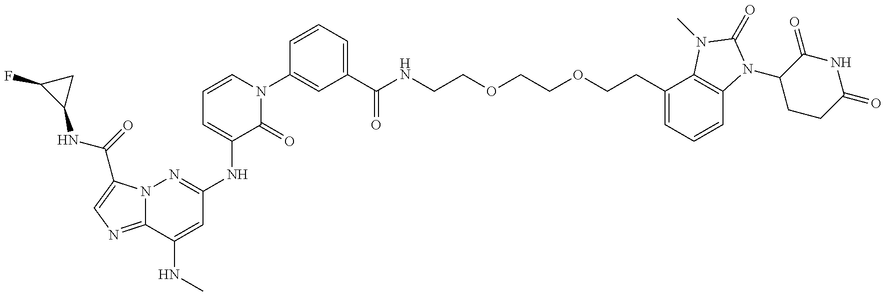 [SMILES: CNc1cc(Nc2cccn(-c3cccc(C(=O)NCCOCCOCCc4cccc5c4n(C)c(=O)n5C4CCC(=O)NC4=O)c3)c2=O)nn2c(C(=O)N[C@@H]3C[C@@H]3F)cnc12] |
| K237 | 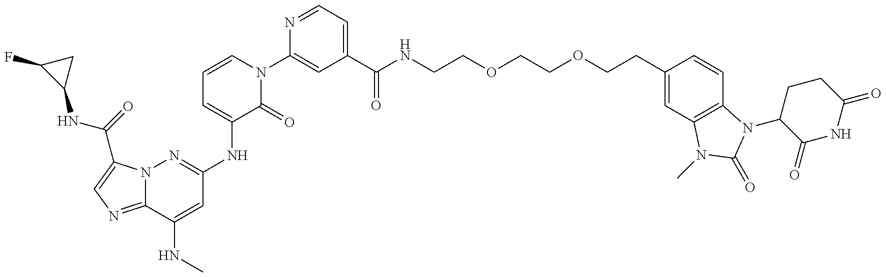 [SMILES: CNc1cc(Nc2cccn(-c3cc(C(=O)NCCOCCOCCc4ccc5c(c4)n(C)c(=O)n5C4CCC(=O)NC4=O)ccn3)c2=O)nn2c(C(=O)N[C@@H]3C[C@@H]3F)cnc12] |
| K238 | 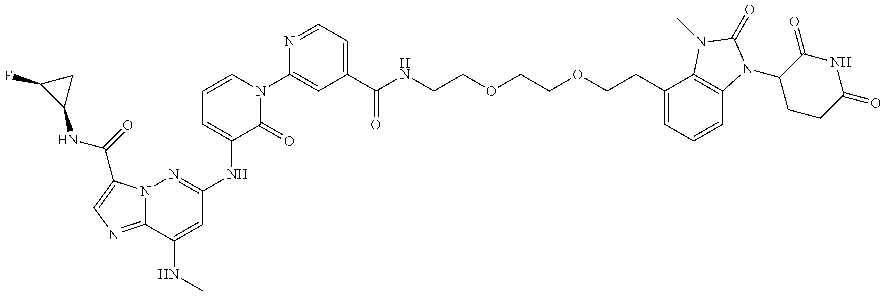 [SMILES: CNc1cc(Nc2cccn(-c3cc(C(=O)NCCOCCOCCc4cccc5c4n(C)c(=O)n5C4CCC(=O)NC4=O)ccn3)c2=O)nn2c(C(=O)N[C@@H]3C[C@@H]3F)cnc12] |
| K239 | 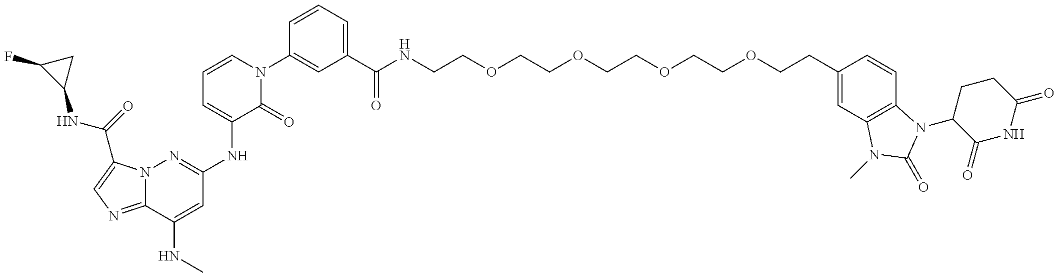 [SMILES: CNc1cc(Nc2cccn(-c3cccc(C(=O)NCCOCCOCCOCCOCCc4ccc5c(c4)n(C)c(=O)n5C4CCC(=O)NC4=O)c3)c2=O)nn2c(C(=O)N[C@@H]3C[C@@H]3F)cnc12] |
| K240 | 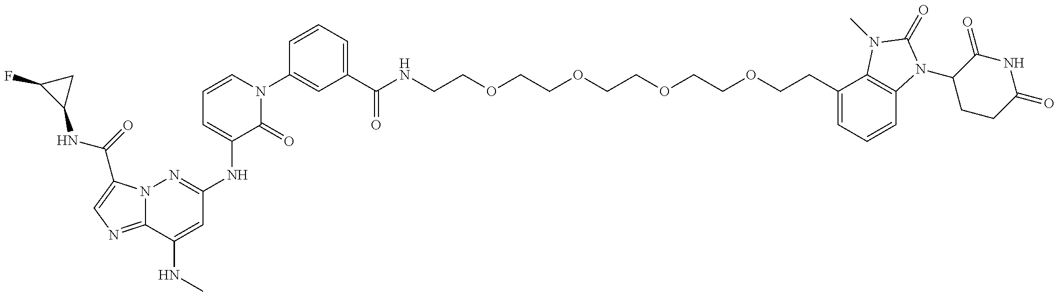 [SMILES: CNc1cc(Nc2cccn(-c3cccc(C(=O)NCCOCCOCCOCCOCCc4cccc5c4n(C)c(=O)n5C4CCC(=O)NC4=O)c3)c2=O)nn2c(C(=O)N[C@@H]3C[C@@H]3F)cnc12] |
| K241 | 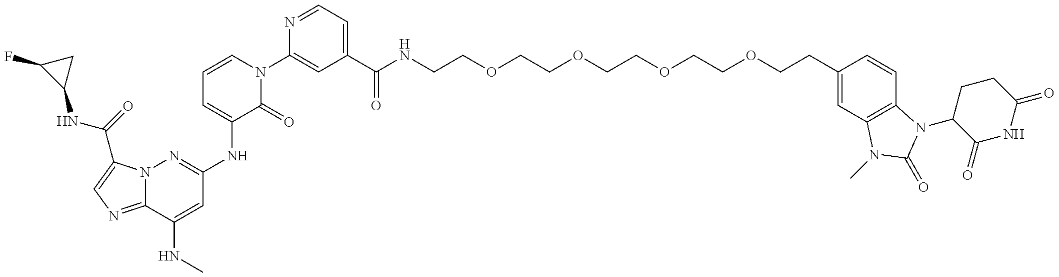 [SMILES: CNc1cc(Nc2cccn(-c3cc(C(=O)NCCOCCOCCOCCOCCc4ccc5c(c4)n(C)c(=O)n5C4CCC(=O)NC4=O)ccn3)c2=O)nn2c(C(=O)N[C@@H]3C[C@@H]3F)cnc12] |
| K242 | 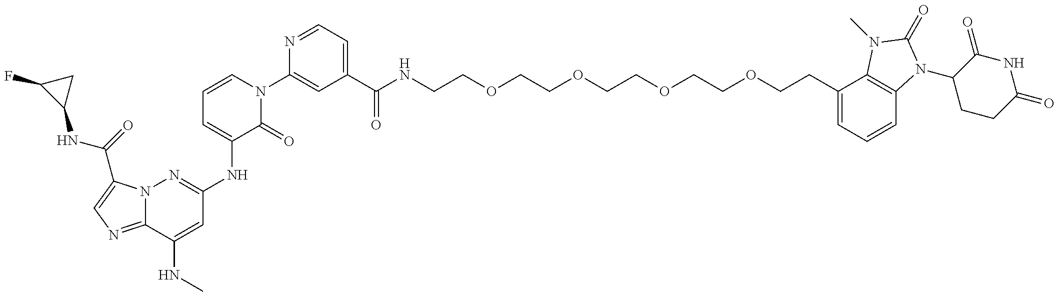 [SMILES: CNc1cc(Nc2cccn(-c3cc(C(=O)NCCOCCOCCOCCOCCc4cccc5c4n(C)c(=O)n5C4CCC(=O)NC4=O)ccn3)c2=O)nn2c(C(=O)N[C@@H]3C[C@@H]3F)cnc12] |
| K243 | 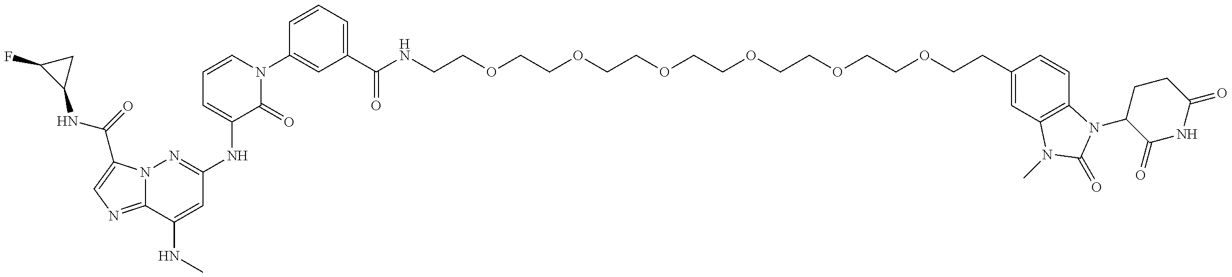 [SMILES: CNc1cc(Nc2cccn(-c3cccc(C(=O)NCCOCCOCCOCCOCCOCCOCCc4ccc5c(c4)n(C)c(=O)n5C4CCC(=O)NC4=O)c3)c2=O)nn2c(C(=O)N[C@@H]3C[C@@H]3F)cnc12] |
| K244 | 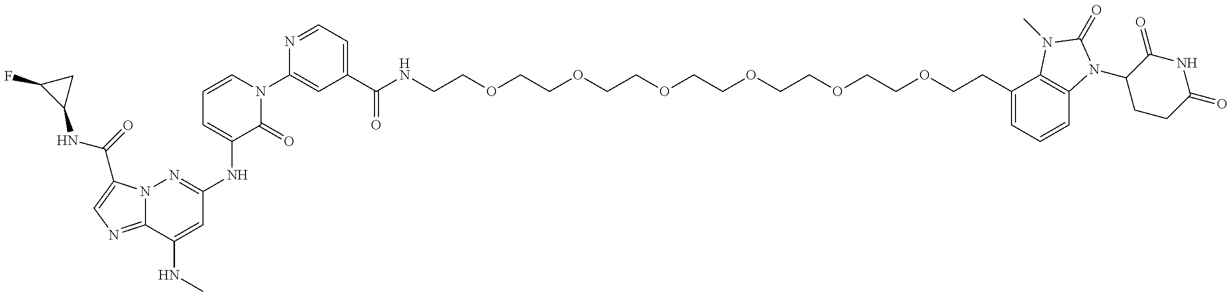 [SMILES: CNc1cc(Nc2cccn(-c3cc(C(=O)NCCOCCOCCOCCOCCOCCOCCc4cccc5c4n(C)c(=O)n5C4CCC(=O)NC4=O)ccn3)c2=O)nn2c(C(=O)N[C@@H]3C[C@@H]3F)cnc12] |
| K245 | 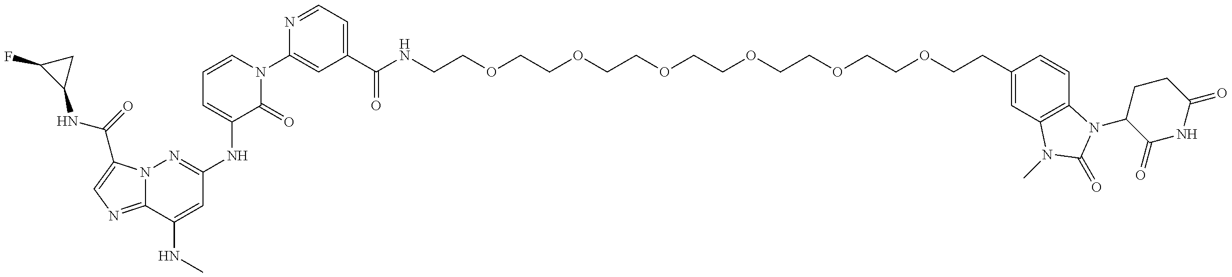 [SMILES: CNc1cc(Nc2cccn(-c3cc(C(=O)NCCOCCOCCOCCOCCOCCOCCc4ccc5c(c4)n(C)c(=O)n5C4CCC(=O)NC4=O)ccn3)c2=O)nn2c(C(=O)N[C@@H]3C[C@@H]3F)cnc12] |
| K246 | 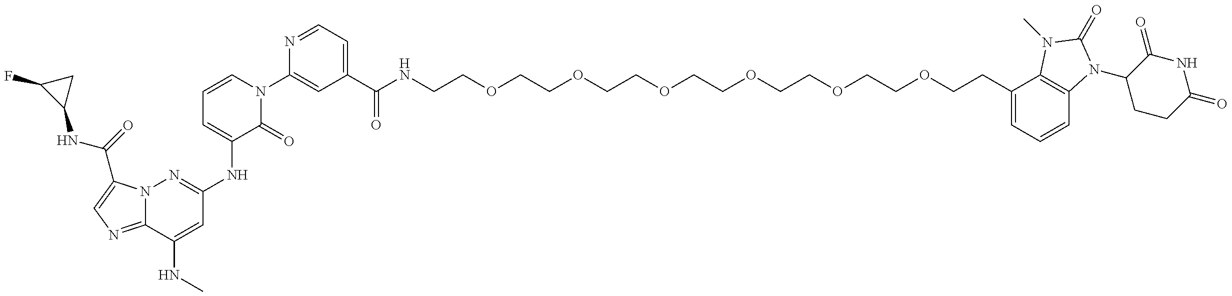 [SMILES: CNc1cc(Nc2cccn(-c3cc(C(=O)NCCOCCOCCOCCOCCOCCOCCc4cccc5c4n(C)c(=O)n5C4CCC(=O)NC4=O)ccn3)c2=O)nn2c(C(=O)N[C@@H]3C[C@@H]3F)cnc12] |
| K247 | 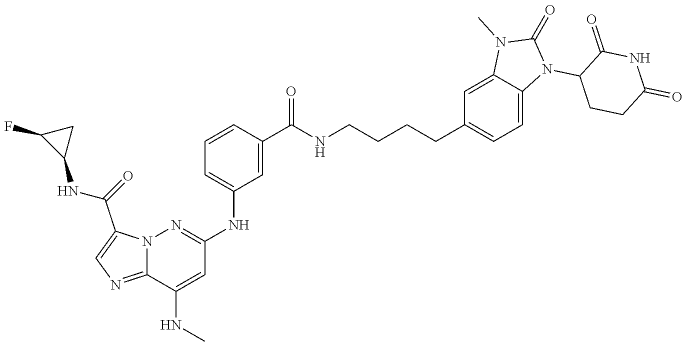 [SMILES: CNc1cc(Nc2cccc(C(=O)NCCCCc3ccc4c(c3)n(C)c(=O)n4C3CCC(=O)NC3=O)c2)nn2c(C(=O)N[C@@H]3C[C@@H]3F)cnc12] |
| K248 | 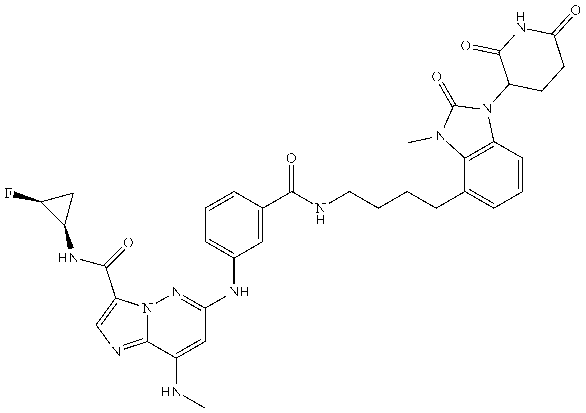 [SMILES: CNc1cc(Nc2cccc(C(=O)NCCCCc3cccc4c3n(C)c(=O)n4C3CCC(=O)NC3=O)c2)nn2c(C(=O)N[C@@H]3C[C@@H]3F)cnc12] |
| K249 | 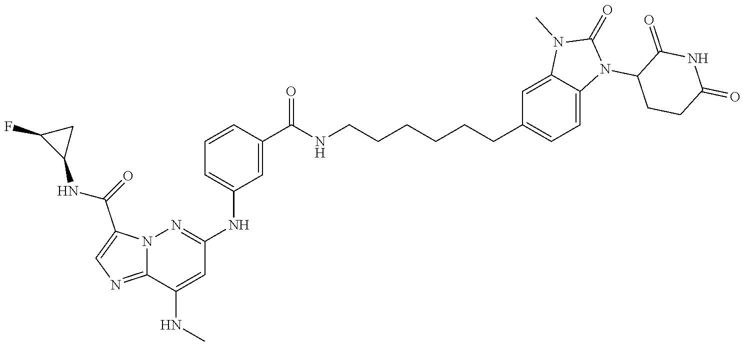 [SMILES: CNc1cc(Nc2cccc(C(=O)NCCCCCCc3ccc4c(c3)n(C)c(=O)n4C3CCC(=O)NC3=O)c2)nn2c(C(=O)N[C@@H]3C[C@@H]3F)cnc12] |
| K250 | 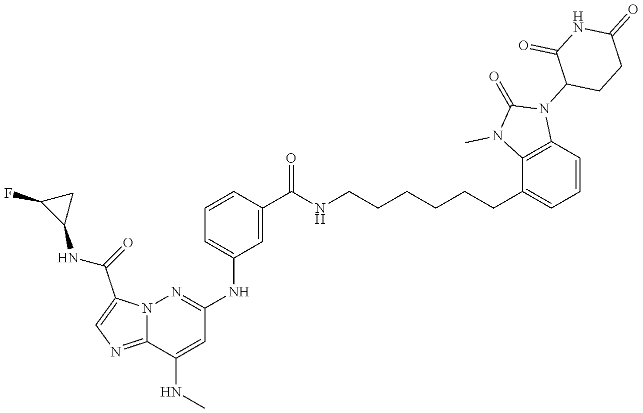 [SMILES: CNc1cc(Nc2cccc(C(=O)NCCCCCCc3cccc4c3n(C)c(=O)n4C3CCC(=O)NC3=O)c2)nn2c(C(=O)N[C@@H]3C[C@@H]3F)cnc12] |
| K251 | 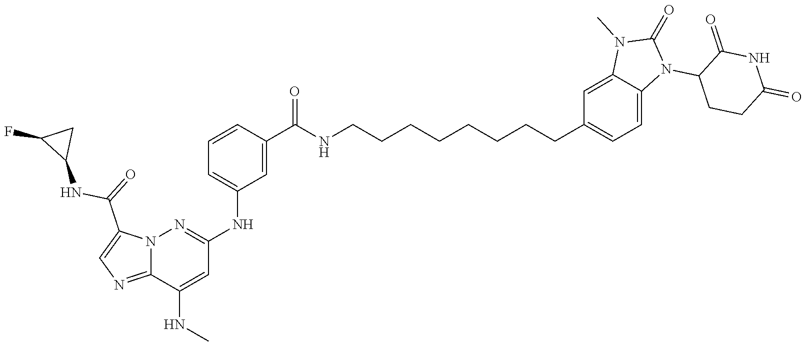 [SMILES: CNc1cc(Nc2cccc(C(=O)NCCCCCCCCc3ccc4c(c3)n(C)c(=O)n4C3CCC(=O)NC3=O)c2)nn2c(C(=O)N[C@@H]3C[C@@H]3F)cnc12] |
| K252 | 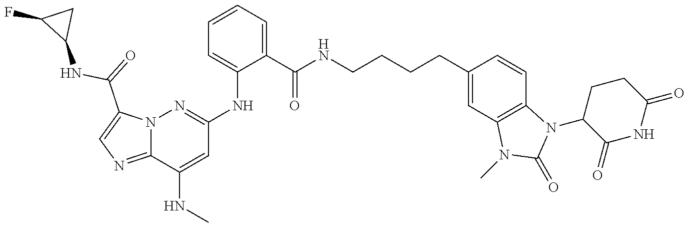 [SMILES: CNc1cc(Nc2ccccc2C(=O)NCCCCc2ccc3c(c2)n(C)c(=O)n3C2CCC(=O)NC2=O)nn2c(C(=O)N[C@@H]3C[C@@H]3F)cnc12] |
| K253 | 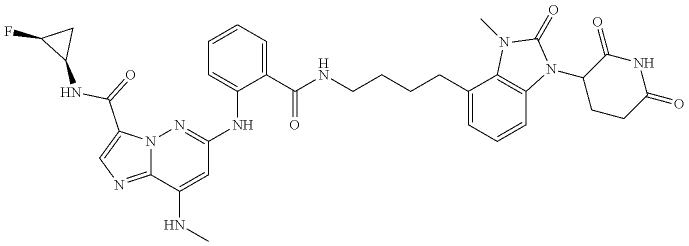 [SMILES: CNc1cc(Nc2ccccc2C(=O)NCCCCc2cccc3c2n(C)c(=O)n3C2CCC(=O)NC2=O)nn2c(C(=O)N[C@@H]3C[C@@H]3F)cnc12] |
| K254 | 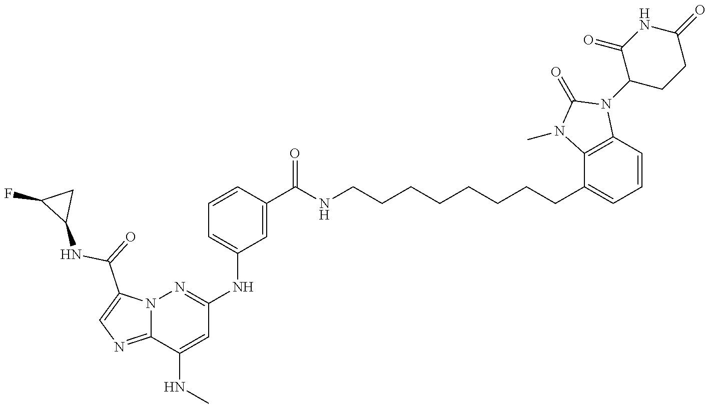 [SMILES: CNc1cc(Nc2cccc(C(=O)NCCCCCCCCc3cccc4c3n(C)c(=O)n4C3CCC(=O)NC3=O)c2)nn2c(C(=O)N[C@@H]3C[C@@H]3F)cnc12] |
| K255 | 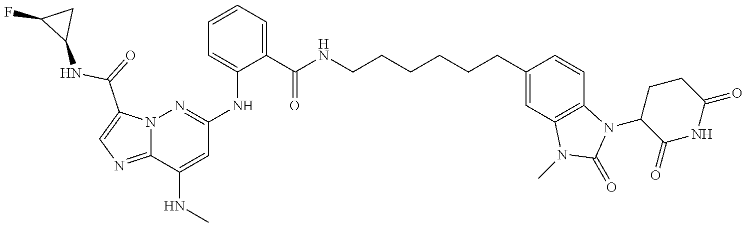 [SMILES: CNc1cc(Nc2ccccc2C(=O)NCCCCCCc2ccc3c(c2)n(C)c(=O)n3C2CCC(=O)NC2=O)nn2c(C(=O)N[C@@H]3C[C@@H]3F)cnc12] |
| K256 | 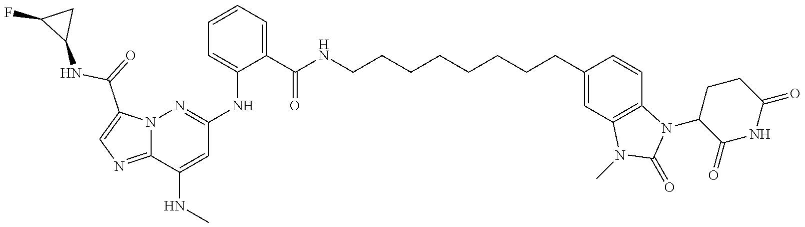 [SMILES: CNc1cc(Nc2ccccc2C(=O)NCCCCCCCCc2ccc3c(c2)n(C)c(=O)n3C2CCC(=O)NC2=O)nn2c(C(=O)N[C@@H]3C[C@@H]3F)cnc12] |
| K257 | 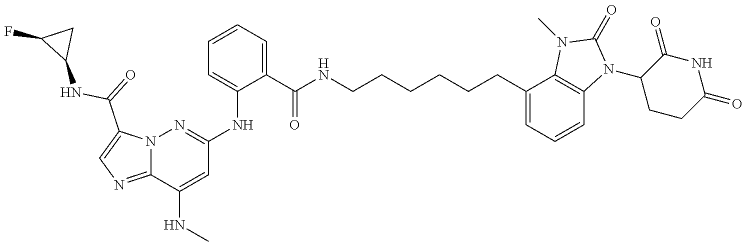 [SMILES: CNc1cc(Nc2ccccc2C(=O)NCCCCCCc2cccc3c2n(C)c(=O)n3C2CCC(=O)NC2=O)nn2c(C(=O)N[C@@H]3C[C@@H]3F)cnc12] |
| K258 | 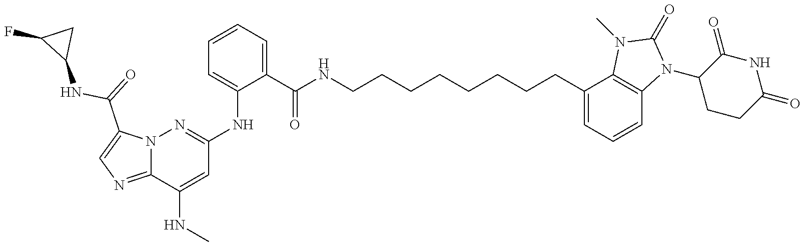 [SMILES: CNc1cc(Nc2ccccc2C(=O)NCCCCCCCCc2cccc3c2n(C)c(=O)n3C2CCC(=O)NC2=O)nn2c(C(=O)N[C@@H]3C[C@@H]3F)cnc12] |
| K259 | 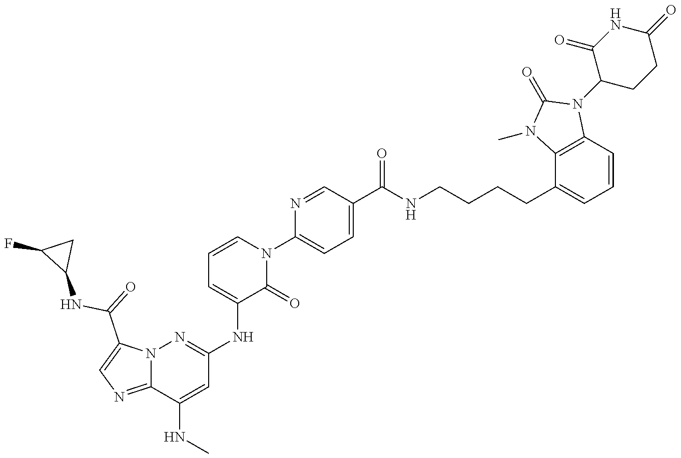 [SMILES: CNc1cc(Nc2cccn(-c3ccc(C(=O)NCCCCc4cccc5c4n(C)c(=O)n5C4CCC(=O)NC4=O)cn3)c2=O)nn2c(C(=O)N[C@@H]3C[C@@H]3F)cnc12] |
| K260 | 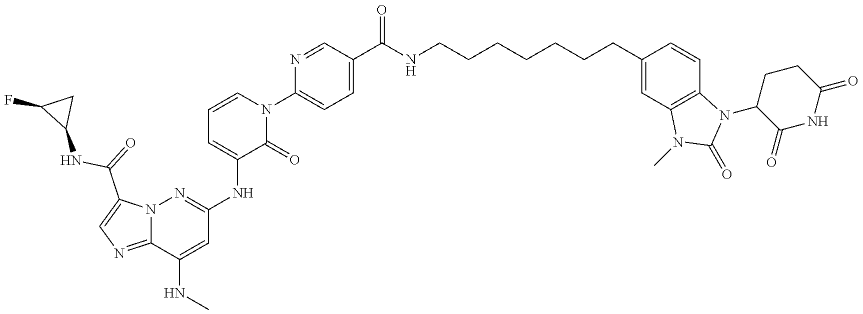 [SMILES: CNc1cc(Nc2cccn(-c3ccc(C(=O)NCCCCCCCc4ccc5c(c4)n(C)c(=O)n5C4CCC(=O)NC4=O)cn3)c2=O)nn2c(C(=O)N[C@@H]3C[C@@H]3F)cnc12] |
| K261 | 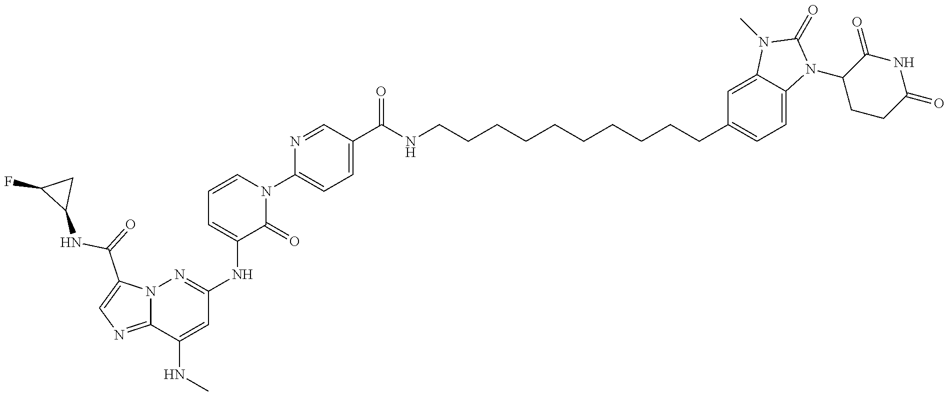 [SMILES: CNc1cc(Nc2cccn(-c3ccc(C(=O)NCCCCCCCCCCc4ccc5c(c4)n(C)c(=O)n5C4CCC(=O)NC4=O)cn3)c2=O)nn2c(C(=O)N[C@@H]3C[C@@H]3F)cnc12] |
| K262 | 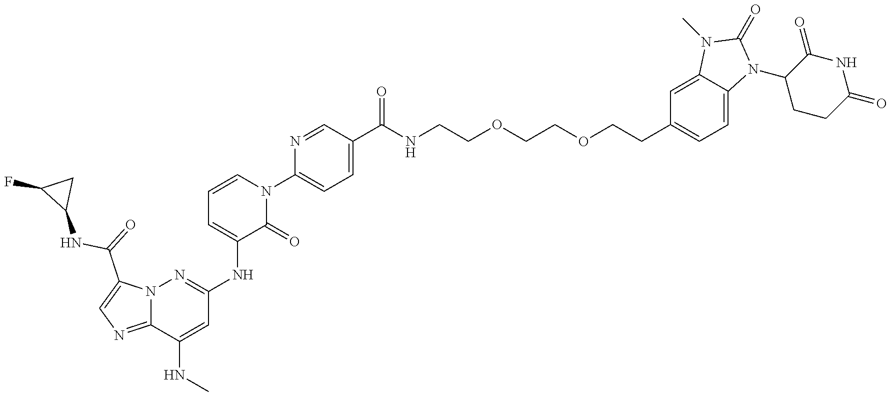 [SMILES: CNc1cc(Nc2cccn(-c3ccc(C(=O)NCCOCCOCCc4ccc5c(c4)n(C)c(=O)n5C4CCC(=O)NC4=O)cn3)c2=O)nn2c(C(=O)N[C@@H]3C[C@@H]3F)cnc12] |
| K263 | 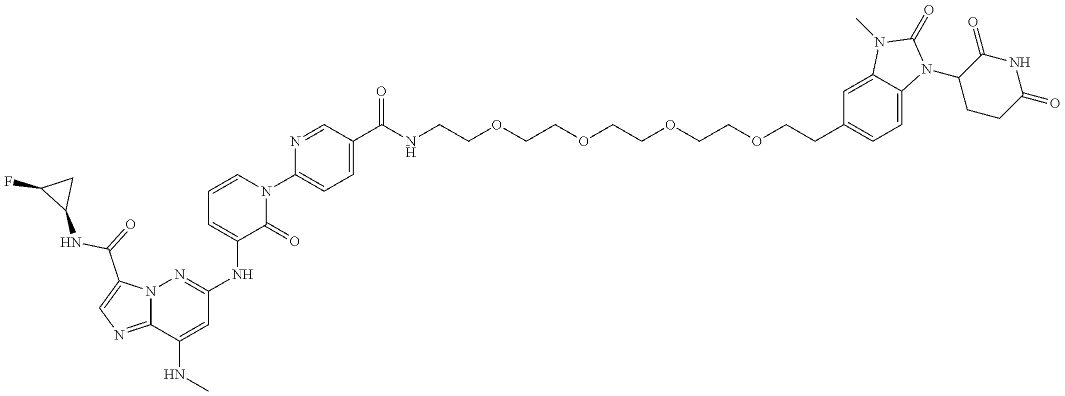 [SMILES: CNc1cc(Nc2cccn(-c3ccc(C(=O)NCCOCCOCCOCCOCCc4ccc5c(c4)n(C)c(=O)n5C4CCC(=O)NC4=O)cn3)c2=O)nn2c(C(=O)N[C@@H]3C[C@@H]3F)cnc12] |
| K264 | 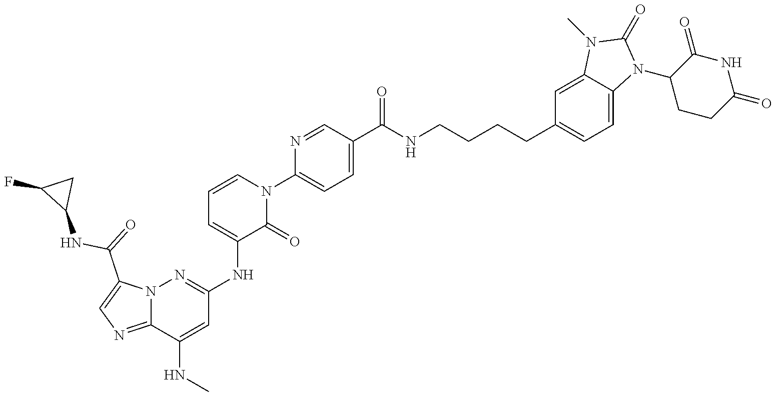 [SMILES: CNc1cc(Nc2cccn(-c3ccc(C(=O)NCCCCc4ccc5c(c4)n(C)c(=O)n5C4CCC(=O)NC4=O)cn3)c2=O)nn2c(C(=O)N[C@@H]3C[C@@H]3F)cnc12] |
| K265 | 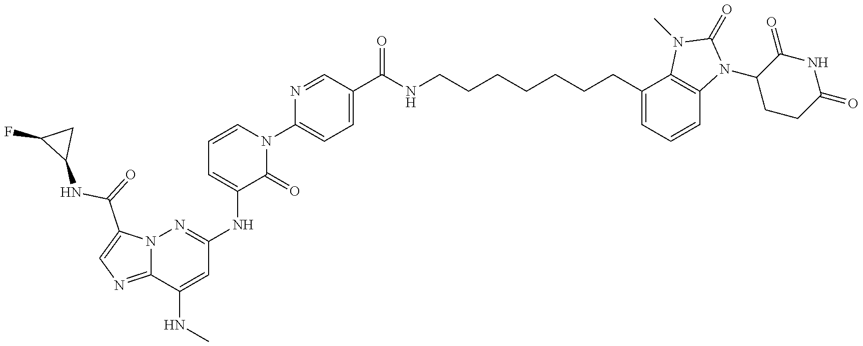 [SMILES: CNc1cc(Nc2cccn(-c3ccc(C(=O)NCCCCCCCc4cccc5c4n(C)c(=O)n5C4CCC(=O)NC4=O)cn3)c2=O)nn2c(C(=O)N[C@@H]3C[C@@H]3F)cnc12] |
| K266 | 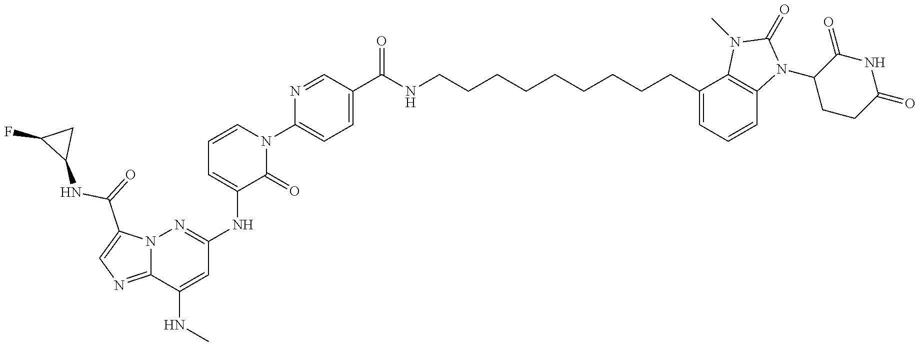 [SMILES: CNc1cc(Nc2cccn(-c3ccc(C(=O)NCCCCCCCCCc4cccc5c4n(C)c(=O)n5C4CCC(=O)NC4=O)cn3)c2=O)nn2c(C(=O)N[C@@H]3C[C@@H]3F)cnc12] |
| K267 | 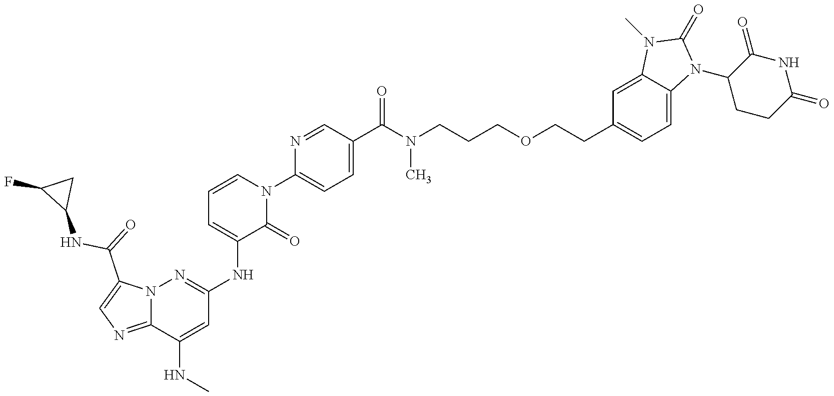 [SMILES: CNc1cc(Nc2cccn(-c3ccc(C(=O)N(C)CCCOCCc4ccc5c(c4)n(C)c(=O)n5C4CCC(=O)NC4=O)cn3)c2=O)nn2c(C(=O)N[C@@H]3C[C@@H]3F)cnc12] |
| K268 | 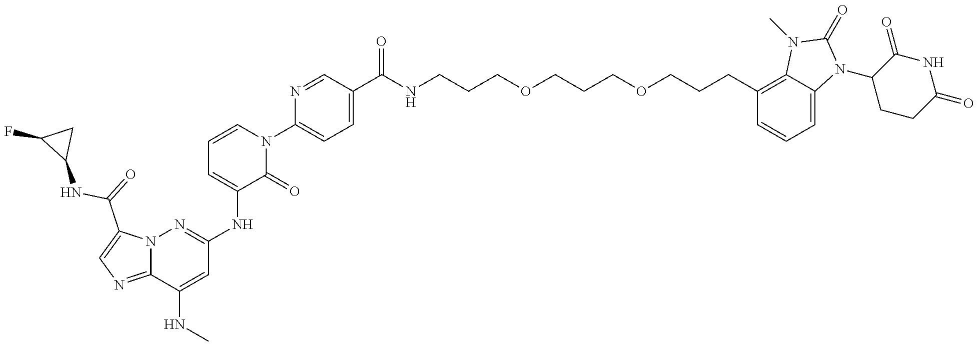 [SMILES: CNc1cc(Nc2cccn(-c3ccc(C(=O)NCCCOCCCOCCCc4cccc5c4n(C)c(=O)n5C4CCC(=O)NC4=O)cn3)c2=O)nn2c(C(=O)N[C@@H]3C[C@@H]3F)cnc12] |
| K269 | 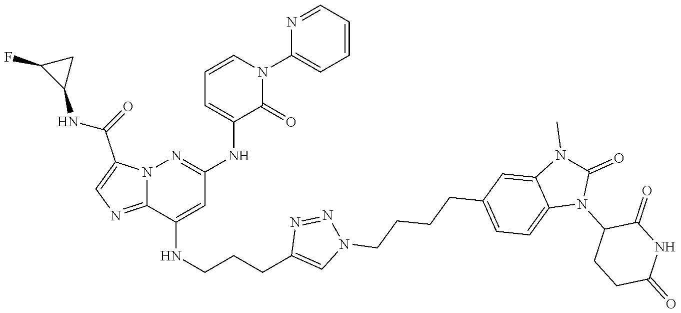 [SMILES: Cn1c(=O)n(C2CCC(=O)NC2=O)c2ccc(CCCCn3cc(CCCNc4cc(Nc5cccn(-c6ccccn6)c5=O)nn5c(C(=O)N[C@@H]6C[C@@H]6F)cnc45)nn3)cc21] |
| K270 | 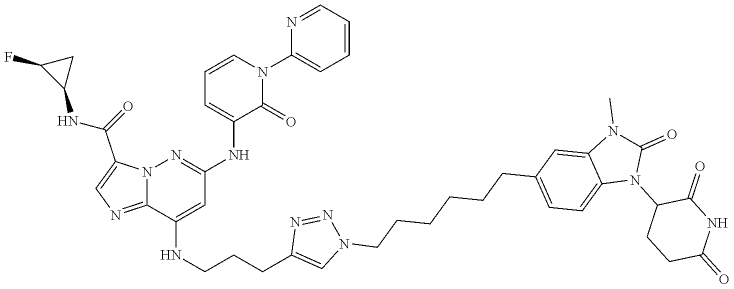 [SMILES: Cn1c(=O)n(C2CCC(=O)NC2=O)c2ccc(CCCCCCn3cc(CCCNc4cc(Nc5cccn(-c6ccccn6)c5=O)nn5c(C(=O)N[C@@H]6C[C@@H]6F)cnc45)nn3)cc21] |
| K271 | 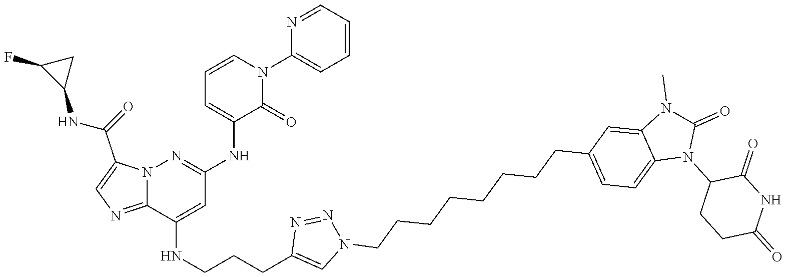 [SMILES: Cn1c(=O)n(C2CCC(=O)NC2=O)c2ccc(CCCCCCCCn3cc(CCCNc4cc(Nc5cccn(-c6ccccn6)c5=O)nn5c(C(=O)N[C@@H]6C[C@@H]6F)cnc45)nn3)cc21] |
| K272 | 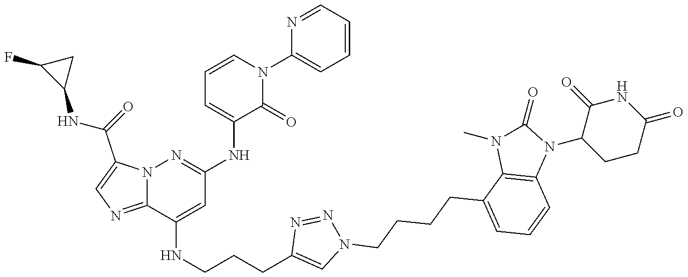 [SMILES: Cn1c(=O)n(C2CCC(=O)NC2=O)c2cccc(CCCCn3cc(CCCNc4cc(Nc5cccn(-c6ccccn6)c5=O)nn5c(C(=O)N[C@@H]6C[C@@H]6F)cnc45)nn3)c21] |
| K273 | 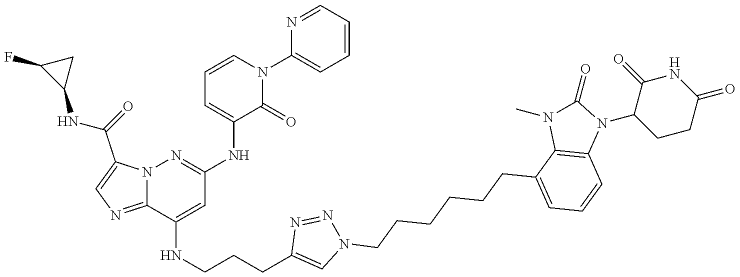 [SMILES: Cn1c(=O)n(C2CCC(=O)NC2=O)c2cccc(CCCCCCn3cc(CCCNc4cc(Nc5cccn(-c6ccccn6)c5=O)nn5c(C(=O)N[C@@H]6C[C@@H]6F)cnc45)nn3)c21] |
| K274 | 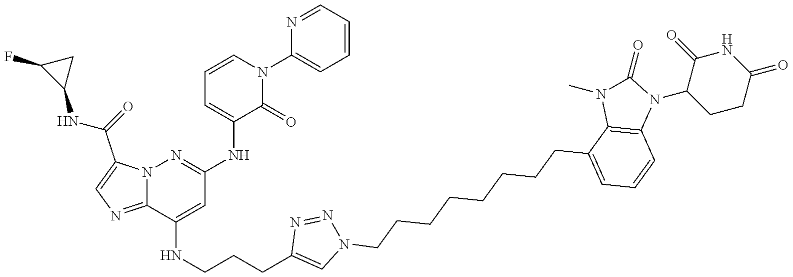 [SMILES: Cn1c(=O)n(C2CCC(=O)NC2=O)c2cccc(CCCCCCCCn3cc(CCCNc4cc(Nc5cccn(-c6ccccn6)c5=O)nn5c(C(=O)N[C@@H]6C[C@@H]6F)cnc45)nn3)c21] |
| K275 | 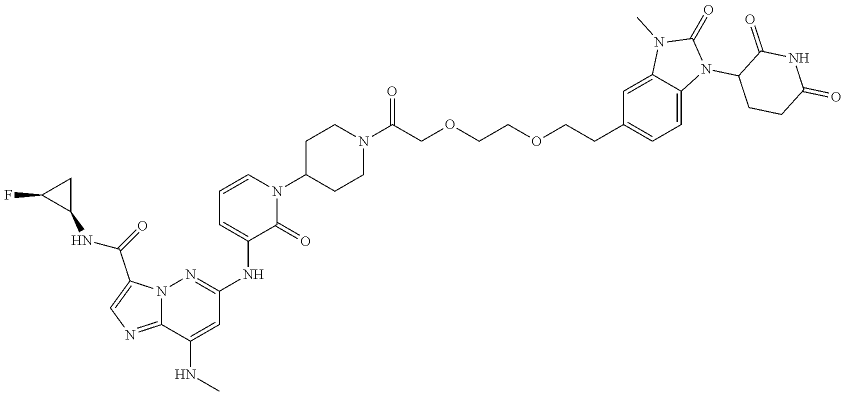 [SMILES: CNc1cc(Nc2cccn(C3CCN(C(=O)COCCOCCc4ccc5c(c4)n(C)c(=O)n5C4CCC(=O)NC4=O)CC3)c2=O)nn2c(C(=O)N[C@@H]3C[C@@H]3F)cnc12] |
| K276 | 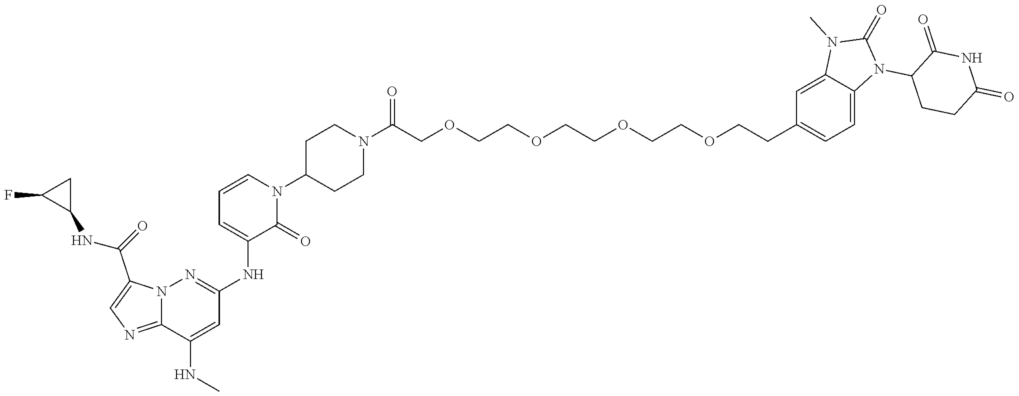 [SMILES: CNc1cc(Nc2cccn(C3CCN(C(=O)COCCOCCOCCOCCc4ccc5c(c4)n(C)c(=O)n5C4CCC(=O)NC4=O)CC3)c2=O)nn2c(C(=O)N[C@@H]3C[C@@H]3F)cnc12] |
| K277 | 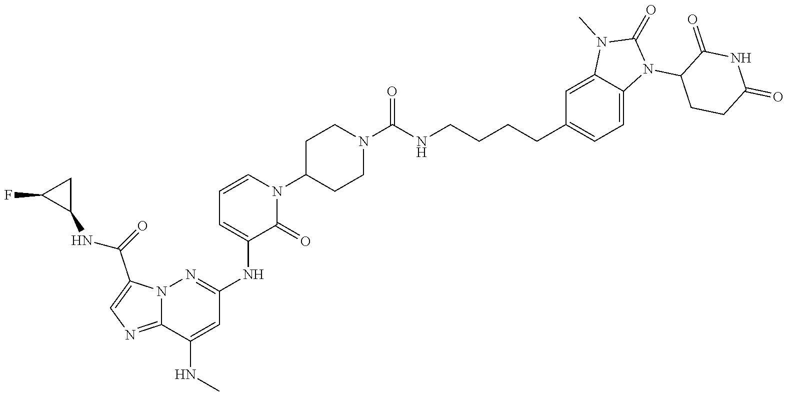 [SMILES: CNc1cc(Nc2cccn(C3CCN(C(=O)NCCCCc4ccc5c(c4)n(C)c(=O)n5C4CCC(=O)NC4=O)CC3)c2=O)nn2c(C(=O)N[C@@H]3C[C@@H]3F)cnc12] |
| K278 | 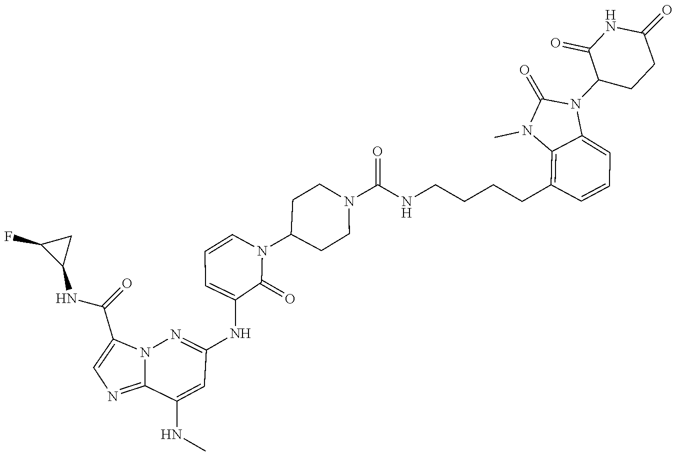 [SMILES: CNc1cc(Nc2cccn(C3CCN(C(=O)NCCCCc4cccc5c4n(C)c(=O)n5C4CCC(=O)NC4=O)CC3)c2=O)nn2c(C(=O)N[C@@H]3C[C@@H]3F)cnc12] |
| K279 | 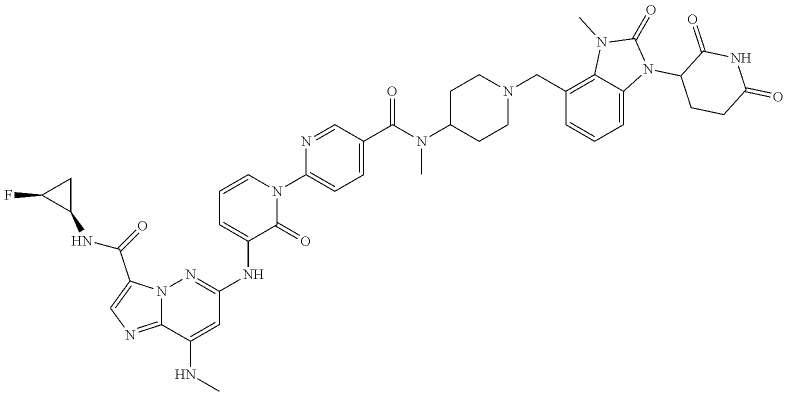 [SMILES: CNc1cc(Nc2cccn(-c3ccc(C(=O)N(C)C4CCN(Cc5cccc6c5n(C)c(=O)n6C5CCC(=O)NC5=O)CC4)cn3)c2=O)nn2c(C(=O)N[C@@H]3C[C@@H]3F)cnc12] |
| K280 | 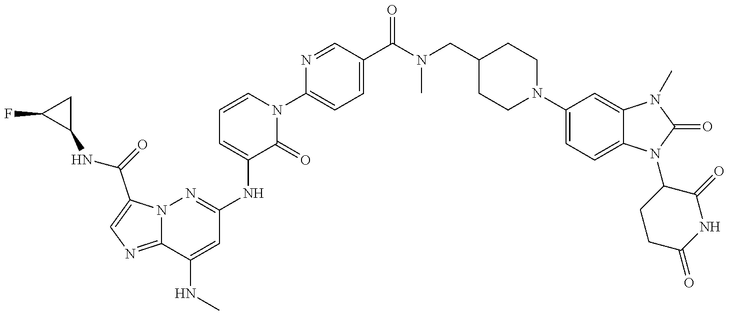 [SMILES: CNc1cc(Nc2cccn(-c3ccc(C(=O)N(C)CC4CCN(c5ccc6c(c5)n(C)c(=O)n6C5CCC(=O)NC5=O)CC4)cn3)c2=O)nn2c(C(=O)N[C@@H]3C[C@@H]3F)cnc12] |
| K281 | 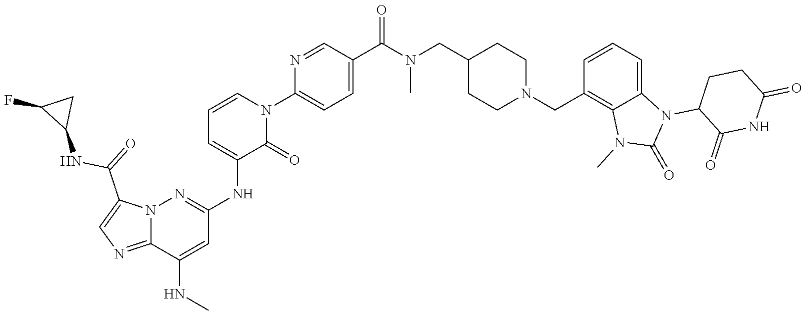 [SMILES: CNc1cc(Nc2cccn(-c3ccc(C(=O)N(C)CC4CCN(Cc5cccc6c5n(C)c(=O)n6C5CCC(=O)NC5=O)CC4)cn3)c2=O)nn2c(C(=O)N[C@@H]3C[C@@H]3F)cnc12] |
| K282 | 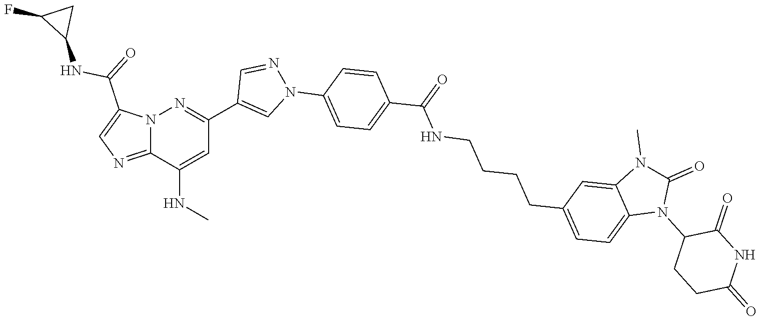 [SMILES: CNc1cc(-c2cnn(-c3ccc(C(=O)NCCCCc4ccc5c(c4)n(C)c(=O)n5C4CCC(=O)NC4=O)cc3)c2)nn2c(C(=O)N[C@@H]3C[C@@H]3F)cnc12] |
| K283 | 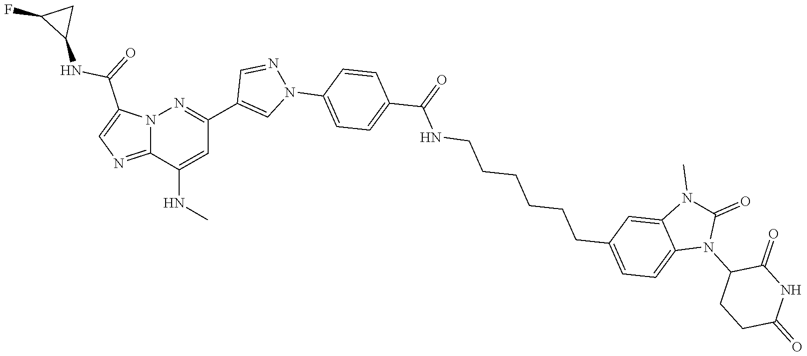 [SMILES: CNc1cc(-c2cnn(-c3ccc(C(=O)NCCCCCCc4ccc5c(c4)n(C)c(=O)n5C4CCC(=O)NC4=O)cc3)c2)nn2c(C(=O)N[C@@H]3C[C@@H]3F)cnc12] |
| K284 | 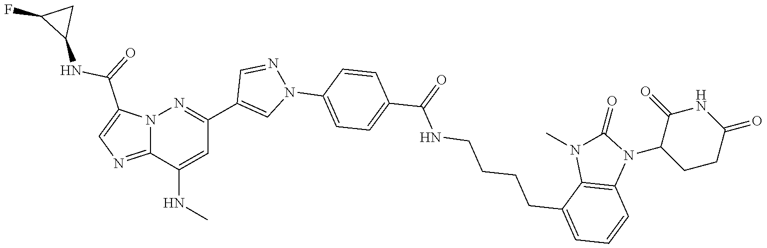 [SMILES: CNc1cc(-c2cnn(-c3ccc(C(=O)NCCCCc4cccc5c4n(C)c(=O)n5C4CCC(=O)NC4=O)cc3)c2)nn2c(C(=O)N[C@@H]3C[C@@H]3F)cnc12] |
| K285 | 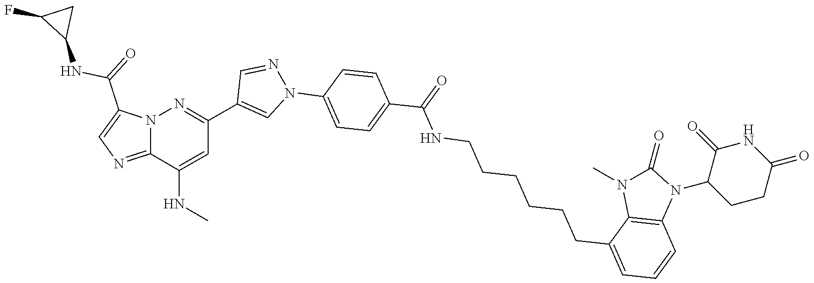 [SMILES: CNc1cc(-c2cnn(-c3ccc(C(=O)NCCCCCCc4cccc5c4n(C)c(=O)n5C4CCC(=O)NC4=O)cc3)c2)nn2c(C(=O)N[C@@H]3C[C@@H]3F)cnc12] |
| K286 | 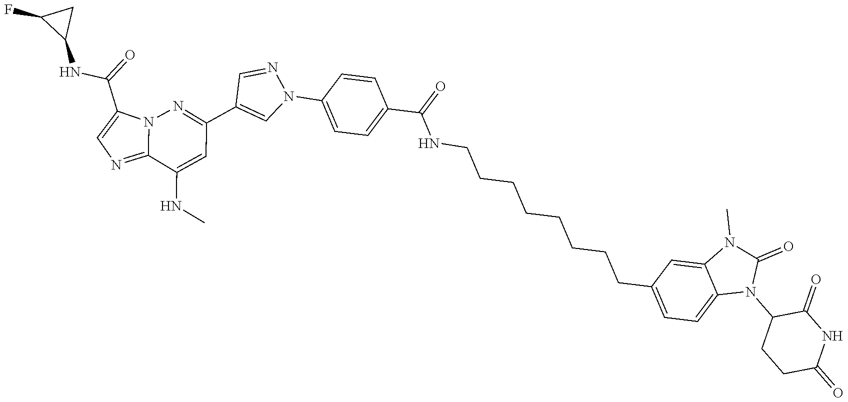 [SMILES: CNc1cc(-c2cnn(-c3ccc(C(=O)NCCCCCCCCc4ccc5c(c4)n(C)c(=O)n5C4CCC(=O)NC4=O)cc3)c2)nn2c(C(=O)N[C@@H]3C[C@@H]3F)cnc12] |
| K287 | 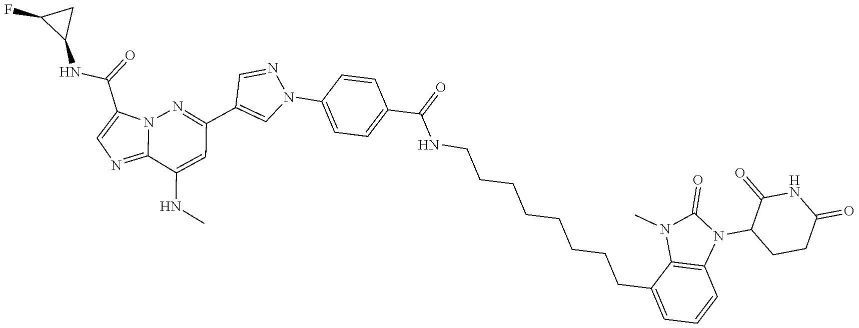 [SMILES: CNc1cc(-c2cnn(-c3ccc(C(=O)NCCCCCCCCc4cccc5c4n(C)c(=O)n5C4CCC(=O)NC4=O)cc3)c2)nn2c(C(=O)N[C@@H]3C[C@@H]3F)cnc12] |
| K288 | 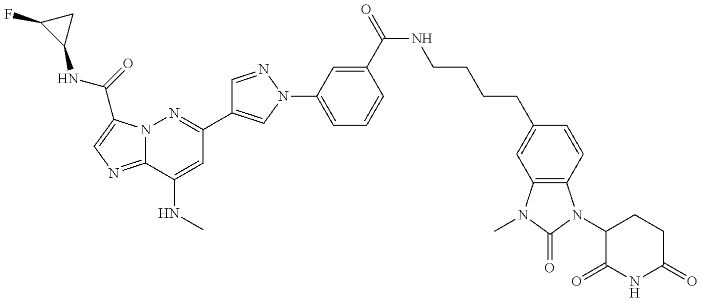 [SMILES: CNc1cc(-c2cnn(-c3cccc(C(=O)NCCCCc4ccc5c(c4)n(C)c(=O)n5C4CCC(=O)NC4=O)c3)c2)nn2c(C(=O)N[C@@H]3C[C@@H]3F)cnc12] |
| K289 | 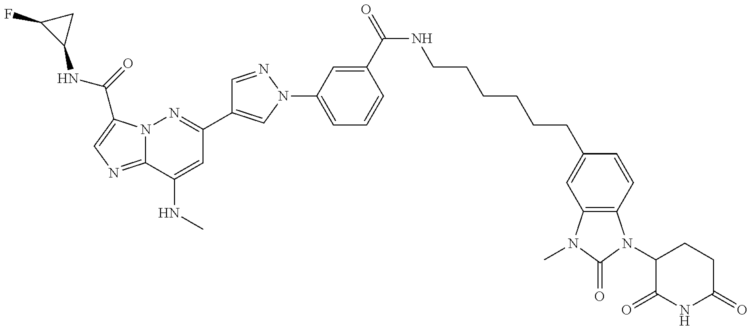 [SMILES: CNc1cc(-c2cnn(-c3cccc(C(=O)NCCCCCCc4ccc5c(c4)n(C)c(=O)n5C4CCC(=O)NC4=O)c3)c2)nn2c(C(=O)N[C@@H]3C[C@@H]3F)cnc12] |
| K290 | 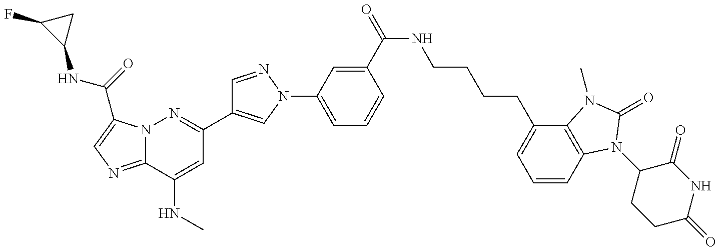 [SMILES: CNc1cc(-c2cnn(-c3cccc(C(=O)NCCCCc4cccc5c4n(C)c(=O)n5C4CCC(=O)NC4=O)c3)c2)nn2c(C(=O)N[C@@H]3C[C@@H]3F)cnc12] |
| K291 | 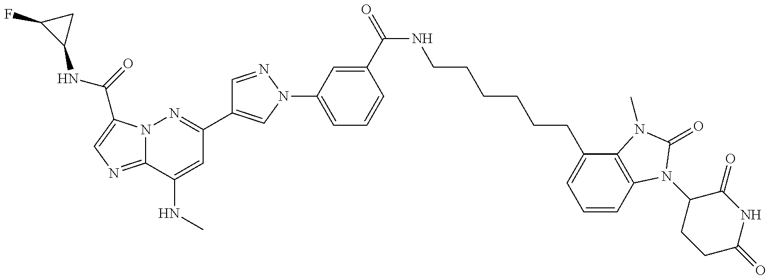 [SMILES: CNc1cc(-c2cnn(-c3cccc(C(=O)NCCCCCCc4cccc5c4n(C)c(=O)n5C4CCC(=O)NC4=O)c3)c2)nn2c(C(=O)N[C@@H]3C[C@@H]3F)cnc12] |
| K292 | 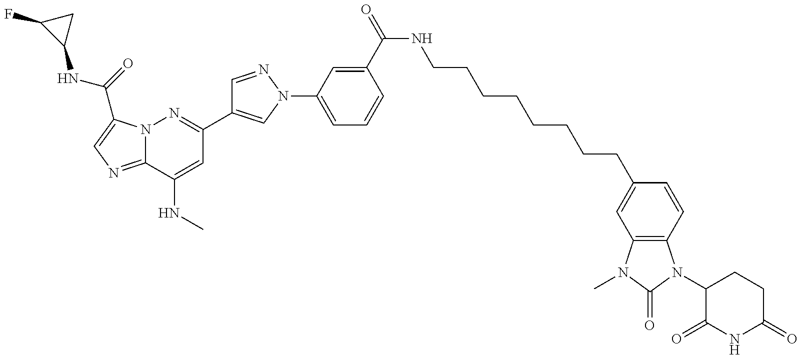 [SMILES: CNc1cc(-c2cnn(-c3cccc(C(=O)NCCCCCCCCc4ccc5c(c4)n(C)c(=O)n5C4CCC(=O)NC4=O)c3)c2)nn2c(C(=O)N[C@@H]3C[C@@H]3F)cnc12] |
| K293 | 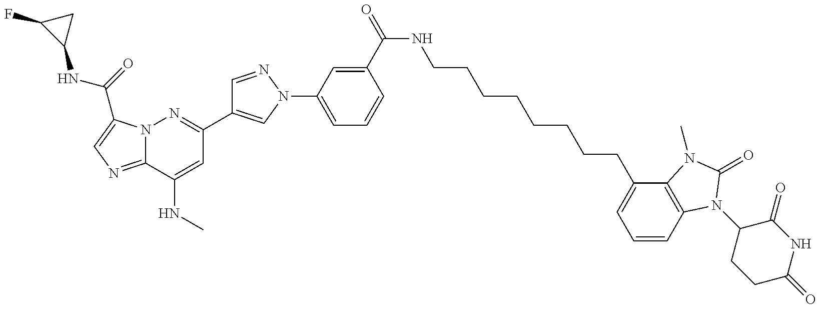 [SMILES: CNc1cc(-c2cnn(-c3cccc(C(=O)NCCCCCCCCc4cccc5c4n(C)c(=O)n5C4CCC(=O)NC4=O)c3)c2)nn2c(C(=O)N[C@@H]3C[C@@H]3F)cnc12] |
| K294 | 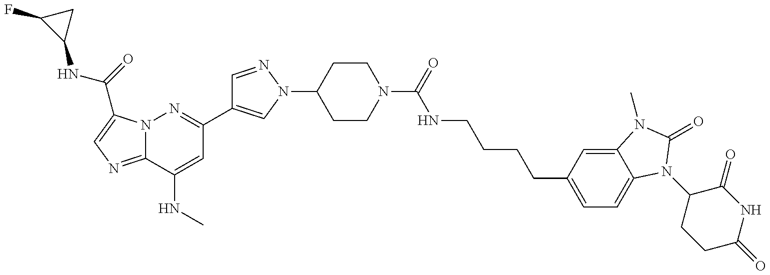 [SMILES: CNc1cc(-c2cnn(C3CCN(C(=O)NCCCCc4ccc5c(c4)n(C)c(=O)n5C4CCC(=O)NC4=O)CC3)c2)nn2c(C(=O)N[C@@H]3C[C@@H]3F)cnc12] |
| K295 | 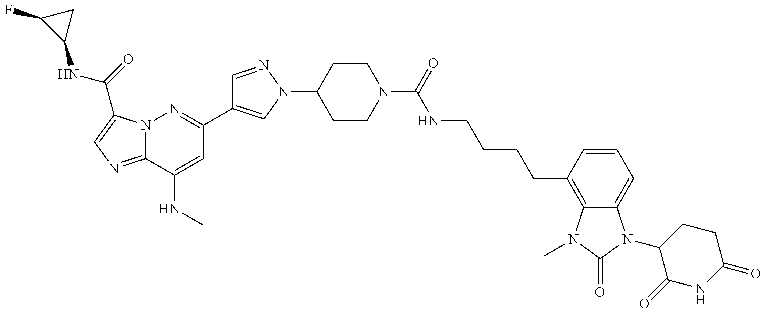 [SMILES: CNc1cc(-c2cnn(C3CCN(C(=O)NCCCCc4cccc5c4n(C)c(=O)n5C4CCC(=O)NC4=O)CC3)c2)nn2c(C(=O)N[C@@H]3C[C@@H]3F)cnc12] |
| K296 | 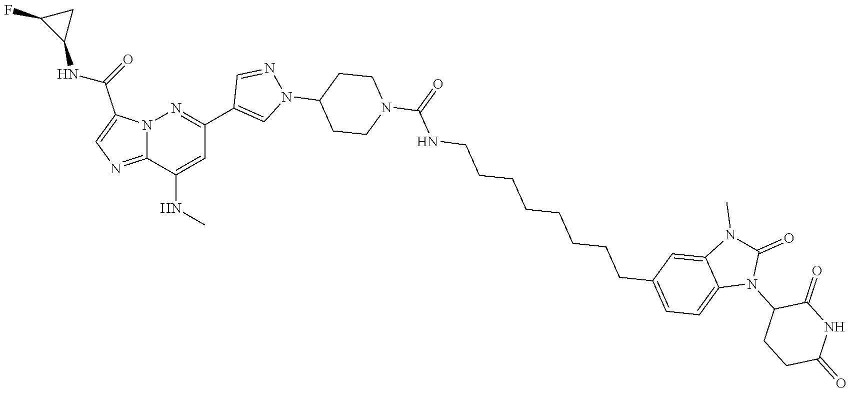 [SMILES: CNc1cc(-c2cnn(C3CCN(C(=O)NCCCCCCCCc4ccc5c(c4)n(C)c(=O)n5C4CCC(=O)NC4=O)CC3)c2)nn2c(C(=O)N[C@@H]3C[C@@H]3F)cnc12] |
| K297 | 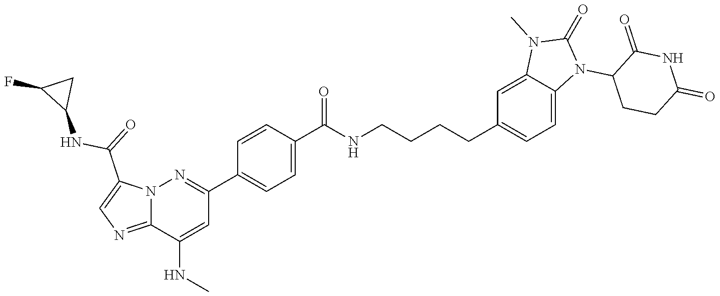 [SMILES: CNc1cc(-c2ccc(C(=O)NCCCCc3ccc4c(c3)n(C)c(=O)n4C3CCC(=O)NC3=O)cc2)nn2c(C(=O)N[C@@H]3C[C@@H]3F)cnc12] |
| K298 | 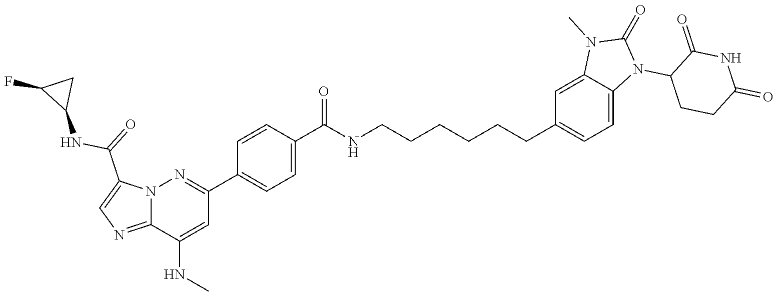 [SMILES: CNc1cc(-c2ccc(C(=O)NCCCCCCc3ccc4c(c3)n(C)c(=O)n4C3CCC(=O)NC3=O)cc2)nn2c(C(=O)N[C@@H]3C[C@@H]3F)cnc12] |
| K299 | 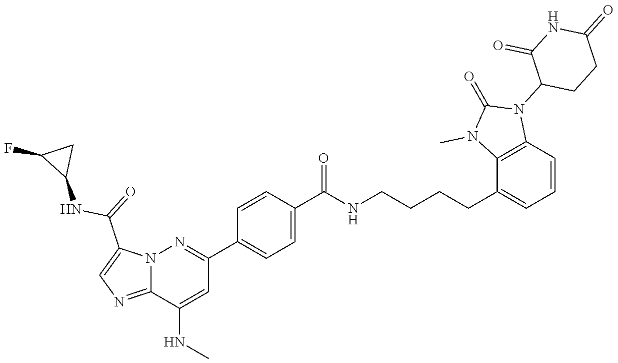 [SMILES: CNc1cc(-c2ccc(C(=O)NCCCCc3cccc4c3n(C)c(=O)n4C3CCC(=O)NC3=O)cc2)nn2c(C(=O)N[C@@H]3C[C@@H]3F)cnc12] |
| K300 | 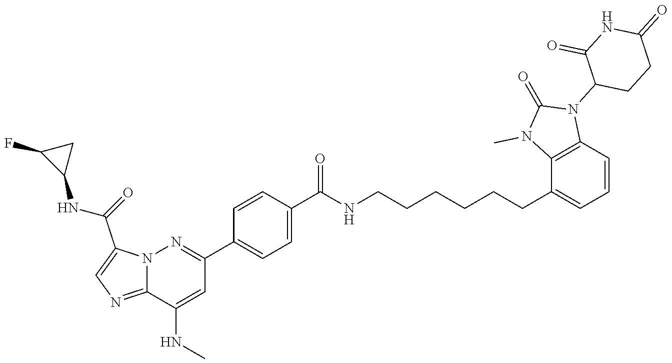 [SMILES: CNc1cc(-c2ccc(C(=O)NCCCCCCc3cccc4c3n(C)c(=O)n4C3CCC(=O)NC3=O)cc2)nn2c(C(=O)N[C@@H]3C[C@@H]3F)cnc12] |
| K301 | 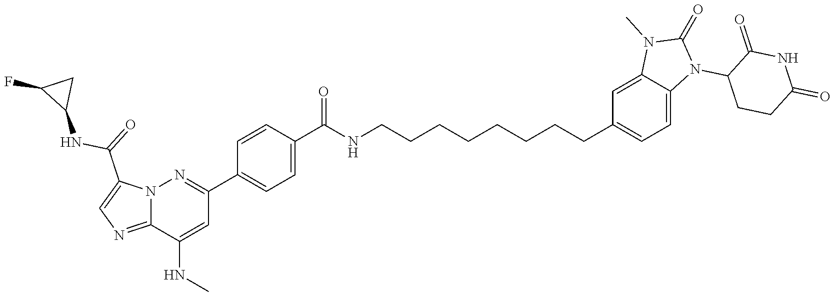 [SMILES: CNc1cc(-c2ccc(C(=O)NCCCCCCCCc3ccc4c(c3)n(C)c(=O)n4C3CCC(=O)NC3=O)cc2)nn2c(C(=O)N[C@@H]3C[C@@H]3F)cnc12] |
| K302 | 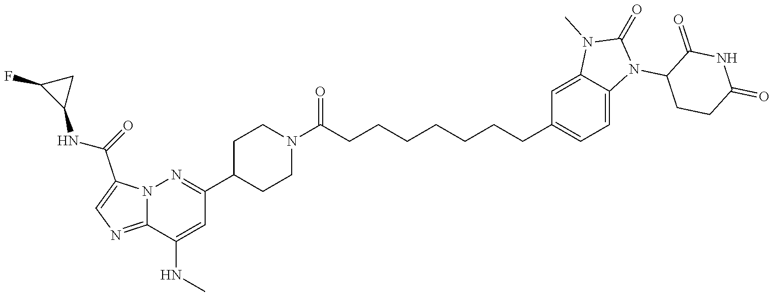 [SMILES: CNc1cc(C2CCN(C(=O)CCCCCCCc3ccc4c(c3)n(C)c(=O)n4C3CCC(=O)NC3=O)CC2)nn2c(C(=O)N[C@@H]3C[C@@H]3F)cnc12] |
| K303 | 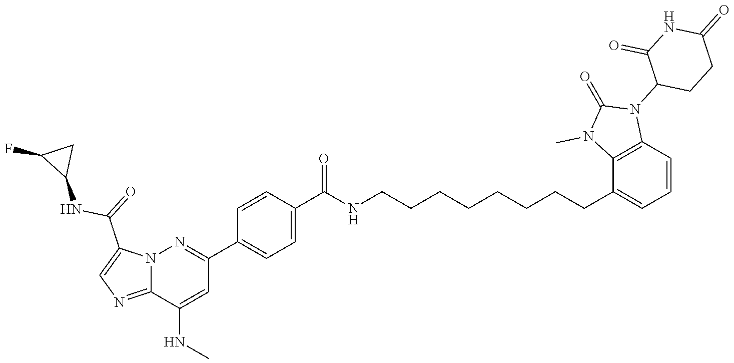 [SMILES: CNc1cc(-c2ccc(C(=O)NCCCCCCCCc3cccc4c3n(C)c(=O)n4C3CCC(=O)NC3=O)cc2)nn2c(C(=O)N[C@@H]3C[C@@H]3F)cnc12] |
| K304 | 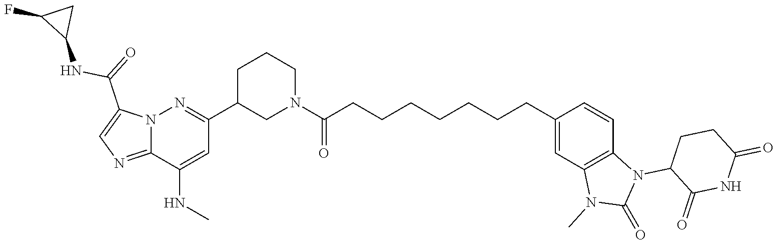 [SMILES: CNc1cc(C2CCCN(C(=O)CCCCCCCc3ccc4c(c3)n(C)c(=O)n4C3CCC(=O)NC3=O)C2)nn2c(C(=O)N[C@@H]3C[C@@H]3F)cnc12] |
| K305 | 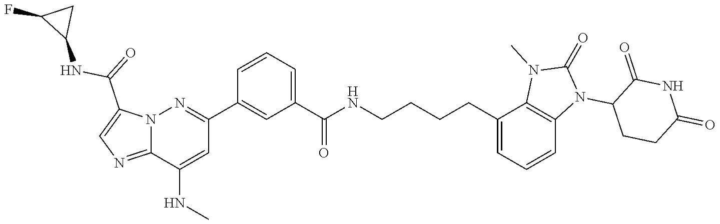 [SMILES: CNc1cc(-c2cccc(C(=O)NCCCCc3cccc4c3n(C)c(=O)n4C3CCC(=O)NC3=O)c2)nn2c(C(=O)N[C@@H]3C[C@@H]3F)cnc12] |
| K306 | 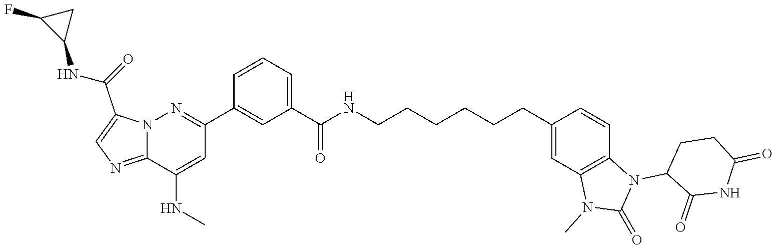 [SMILES: CNc1cc(-c2cccc(C(=O)NCCCCCCc3ccc4c(c3)n(C)c(=O)n4C3CCC(=O)NC3=O)c2)nn2c(C(=O)N[C@@H]3C[C@@H]3F)cnc12] |
| K307 | 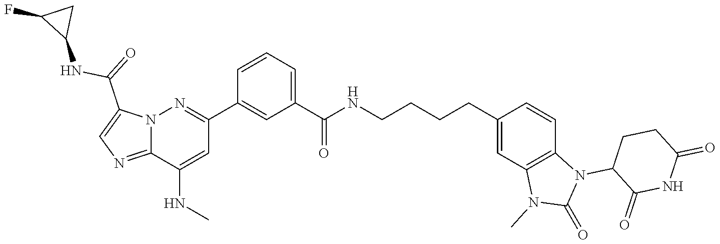 [SMILES: CNc1cc(-c2cccc(C(=O)NCCCCc3ccc4c(c3)n(C)c(=O)n4C3CCC(=O)NC3=O)c2)nn2c(C(=O)N[C@@H]3C[C@@H]3F)cnc12] |
| K308 | 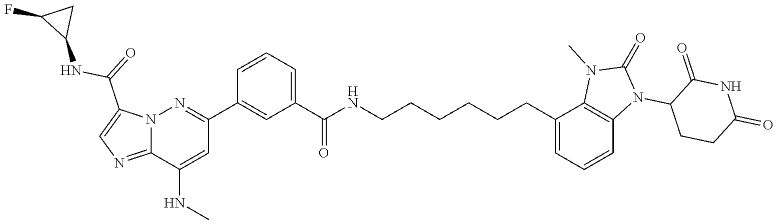 [SMILES: CNc1cc(-c2cccc(C(=O)NCCCCCCc3cccc4c3n(C)c(=O)n4C3CCC(=O)NC3=O)c2)nn2c(C(=O)N[C@@H]3C[C@@H]3F)cnc12] |
| K309 | 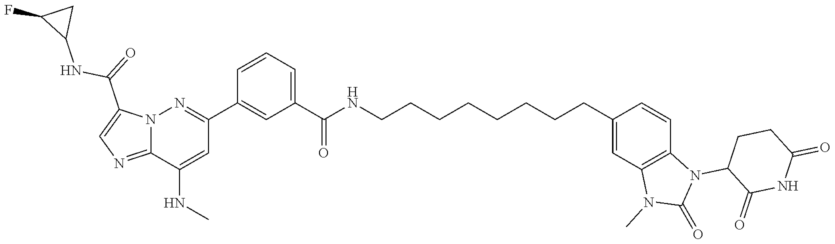 [SMILES: CNc1cc(-c2cccc(C(=O)NCCCCCCCCc3ccc4c(c3)n(C)c(=O)n4C3CCC(=O)NC3=O)c2)nn2c(C(=O)NC3C[C@@H]3F)cnc12] |
| K310 | 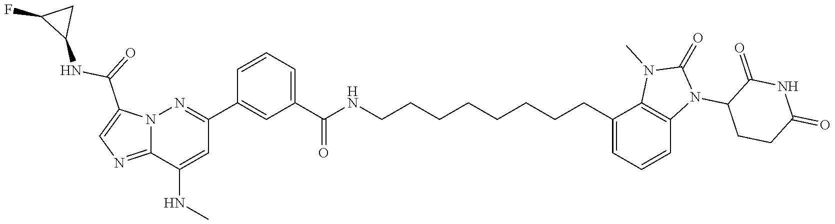 [SMILES: CNc1cc(-c2cccc(C(=O)NCCCCCCCCc3cccc4c3n(C)c(=O)n4C3CCC(=O)NC3=O)c2)nn2c(C(=O)N[C@@H]3C[C@@H]3F)cnc12] |
| K311 | 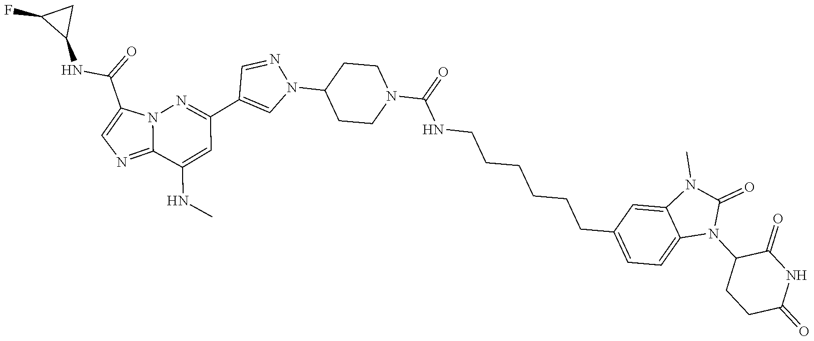 [SMILES: CNc1cc(-c2cnn(C3CCN(C(=O)NCCCCCCc4ccc5c(c4)n(C)c(=O)n5C4CCC(=O)NC4=O)CC3)c2)nn2c(C(=O)N[C@@H]3C[C@@H]3F)cnc12] |
| K312 | 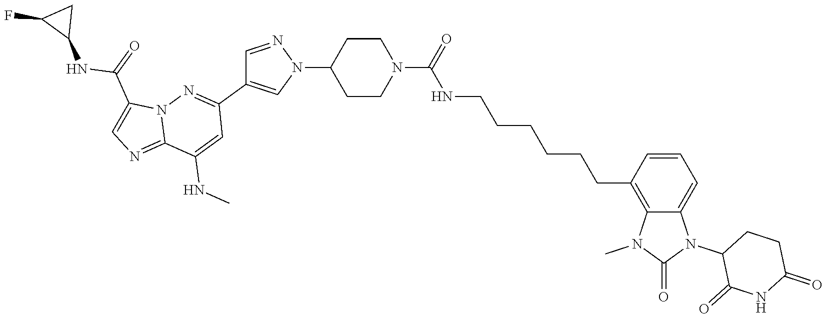 [SMILES: CNc1cc(-c2cnn(C3CCN(C(=O)NCCCCCCc4cccc5c4n(C)c(=O)n5C4CCC(=O)NC4=O)CC3)c2)nn2c(C(=O)N[C@@H]3C[C@@H]3F)cnc12] |
| K313 | 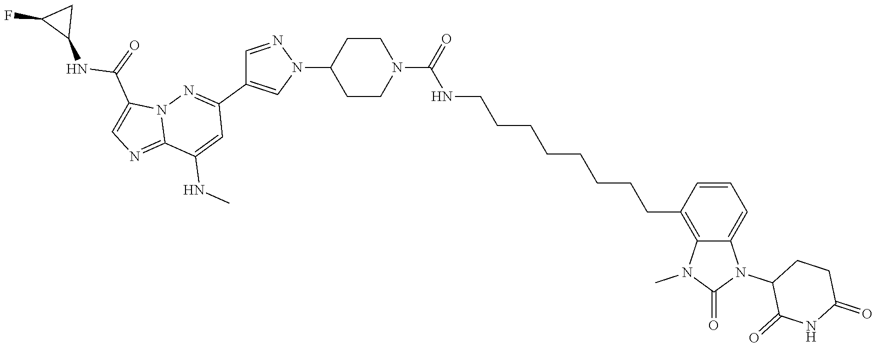 [SMILES: CNc1cc(-c2cnn(C3CCN(C(=O)NCCCCCCCCc4cccc5c4n(C)c(=O)n5C4CCC(=O)NC4=O)CC3)c2)nn2c(C(=O)N[C@@H]3C[C@@H]3F)cnc12] |
| K314 | 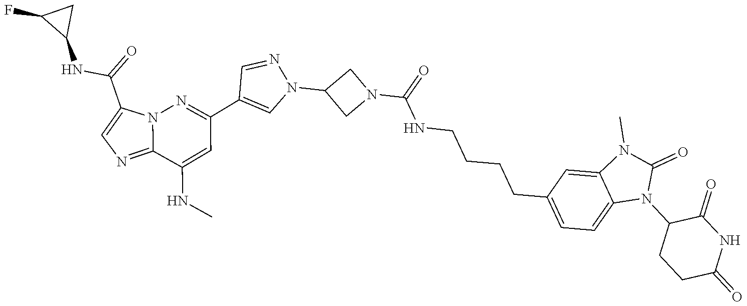 [SMILES: CNc1cc(-c2cnn(C3CN(C(=O)NCCCCc4ccc5c(c4)n(C)c(=O)n5C4CCC(=O)NC4=O)C3)c2)nn2c(C(=O)N[C@@H]3C[C@@H]3F)cnc12] |
| K315 | 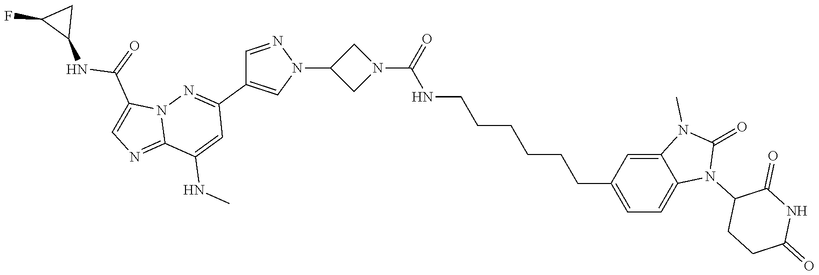 [SMILES: CNc1cc(-c2cnn(C3CN(C(=O)NCCCCCCc4ccc5c(c4)n(C)c(=O)n5C4CCC(=O)NC4=O)C3)c2)nn2c(C(=O)N[C@@H]3C[C@@H]3F)cnc12] |
| K316 | 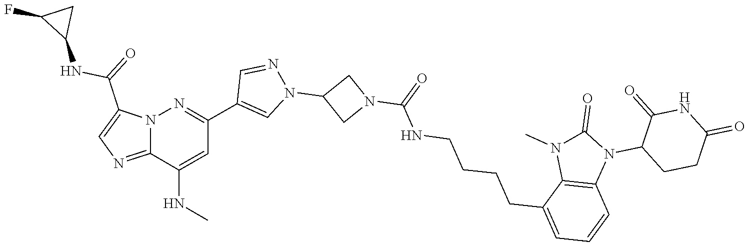 [SMILES: CNc1cc(-c2cnn(C3CN(C(=O)NCCCCc4cccc5c4n(C)c(=O)n5C4CCC(=O)NC4=O)C3)c2)nn2c(C(=O)N[C@@H]3C[C@@H]3F)cnc12] |
| K317 | 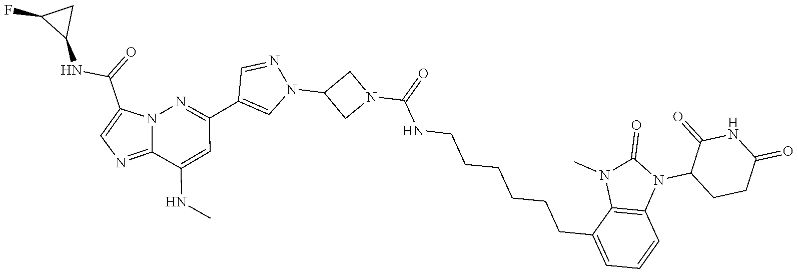 [SMILES: CNc1cc(-c2cnn(C3CN(C(=O)NCCCCCCc4cccc5c4n(C)c(=O)n5C4CCC(=O)NC4=O)C3)c2)nn2c(C(=O)N[C@@H]3C[C@@H]3F)cnc12] |
| K318 | 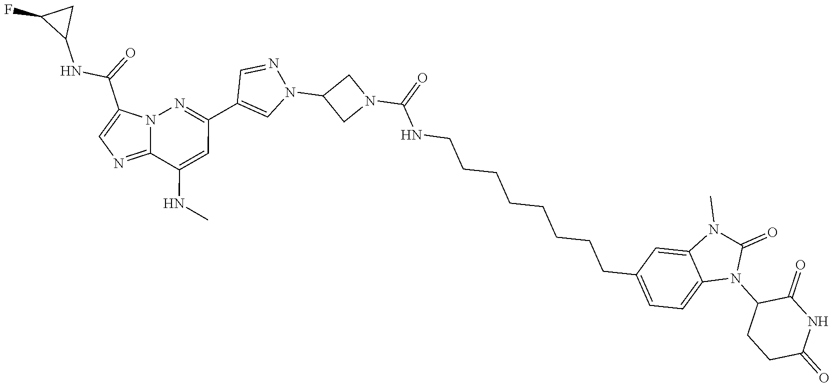 [SMILES: CNc1cc(-c2cnn(C3CN(C(=O)NCCCCCCCCc4ccc5c(c4)n(C)c(=O)n5C4CCC(=O)NC4=O)C3)c2)nn2c(C(=O)NC3C[C@@H]3F)cnc12] |
| K319 | 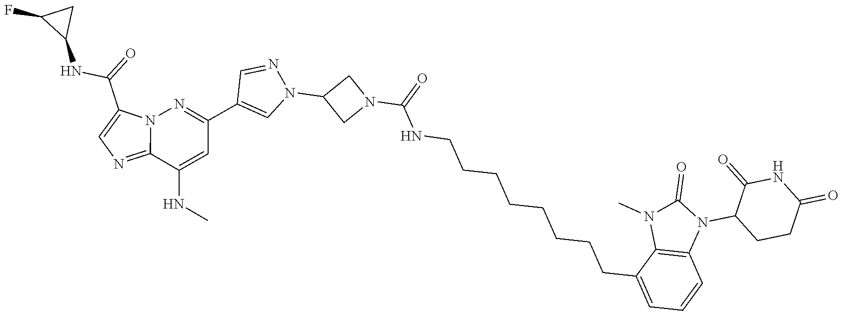 [SMILES: CNc1cc(-c2cnn(C3CN(C(=O)NCCCCCCCCc4cccc5c4n(C)c(=O)n5C4CCC(=O)NC4=O)C3)c2)nn2c(C(=O)N[C@@H]3C[C@@H]3F)cnc12] |
| K320 | 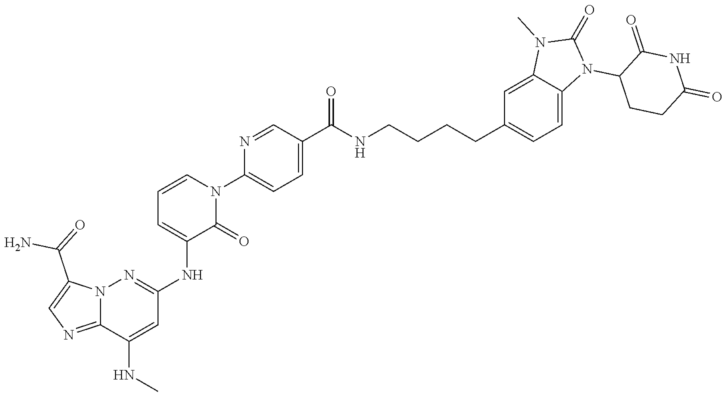 [SMILES: CNc1cc(Nc2cccn(-c3ccc(C(=O)NCCCCc4ccc5c(c4)n(C)c(=O)n5C4CCC(=O)NC4=O)cn3)c2=O)nn2c(C(N)=O)cnc12] |
| K321 | 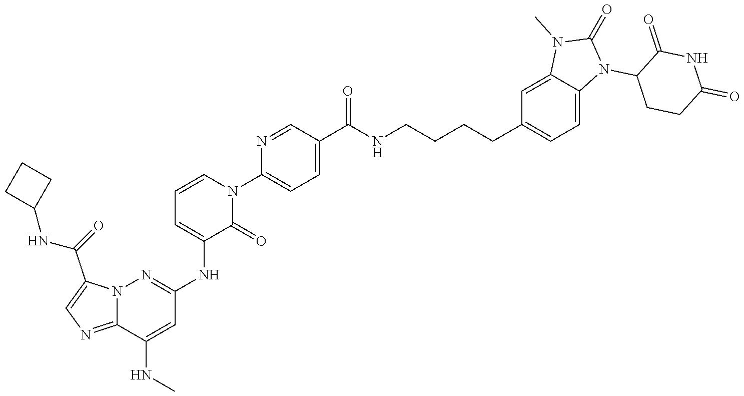 [SMILES: CNc1cc(Nc2cccn(-c3ccc(C(=O)NCCCCc4ccc5c(c4)n(C)c(=O)n5C4CCC(=O)NC4=O)cn3)c2=O)nn2c(C(=O)NC3CCC3)cnc12] |
| K322 | 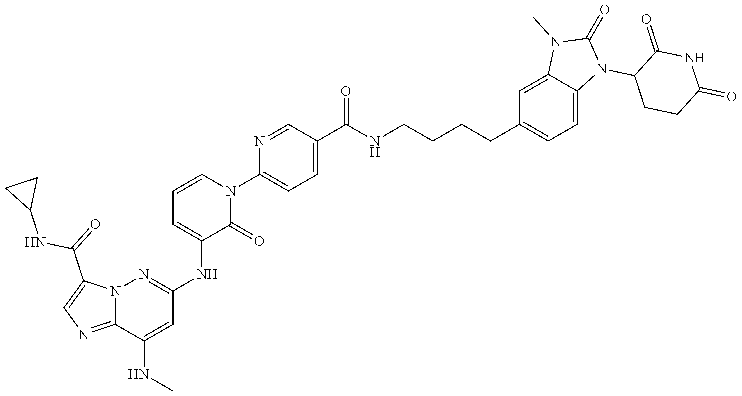 [SMILES: CNc1cc(Nc2cccn(-c3ccc(C(=O)NCCCCc4ccc5c(c4)n(C)c(=O)n5C4CCC(=O)NC4=O)cn3)c2=O)nn2c(C(=O)NC3CC3)cnc12] |
| K323 | 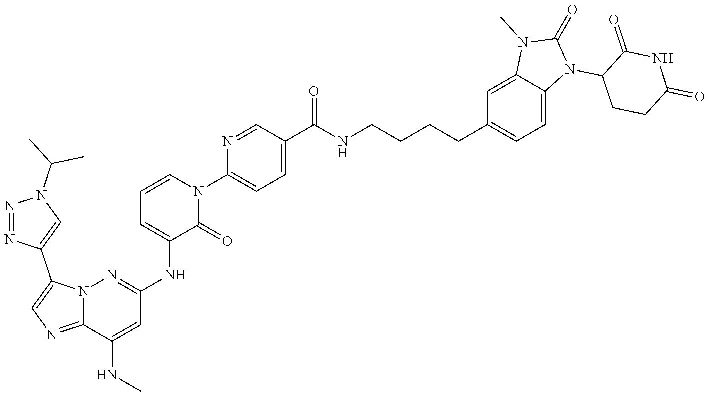 [SMILES: CNc1cc(Nc2cccn(-c3ccc(C(=O)NCCCCc4ccc5c(c4)n(C)c(=O)n5C4CCC(=O)NC4=O)cn3)c2=O)nn2c(-c3cn(C(C)C)nn3)cnc12] |
| K324 | 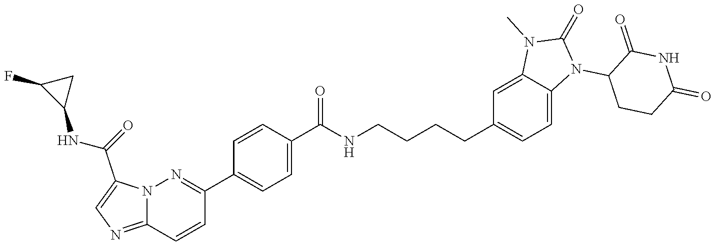 [SMILES: Cn1c(=O)n(C2CCC(=O)NC2=O)c2ccc(CCCCNC(=O)c3ccc(-c4ccc5ncc(C(=O)N[C@@H]6C[C@@H]6F)n5n4)cc3)cc21] |
| K325 | 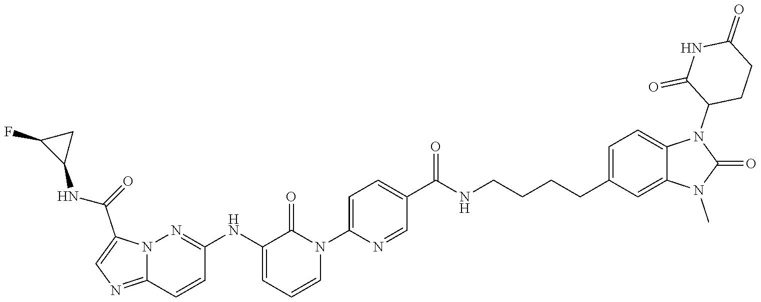 [SMILES: Cn1c(=O)n(C2CCC(=O)NC2=O)c2ccc(CCCCNC(=O)c3ccc(-n4cccc(Nc5ccc6ncc(C(=O)N[C@@H]7C[C@@H]7F)n6n5)c4=O)nc3)cc21] |
| K326 | 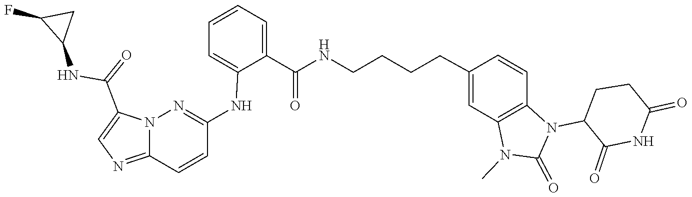 [SMILES: Cn1c(=O)n(C2CCC(=O)NC2=O)c2ccc(CCCCNC(=O)c3ccccc3Nc3ccc4ncc(C(=O)N[C@@H]5C[C@@H]5F)n4n3)cc21] |
| K327 | 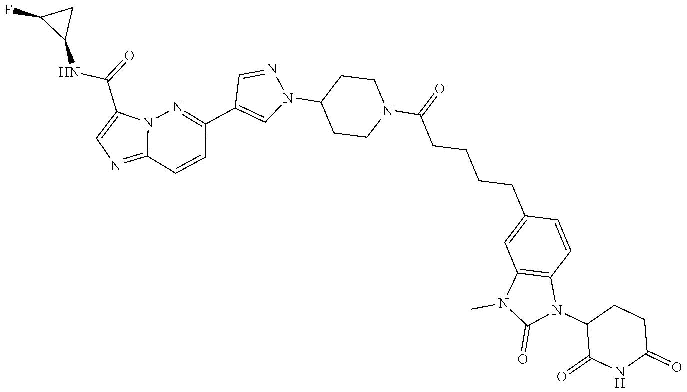 [SMILES: Cn1c(=O)n(C2CCC(=O)NC2=O)c2ccc(CCCCC(=O)N3CCC(n4cc(-c5ccc6ncc(C(=O)N[C@@H]7C[C@@H]7F)n6n5)cn4)CC3)cc21] |
| K328 | 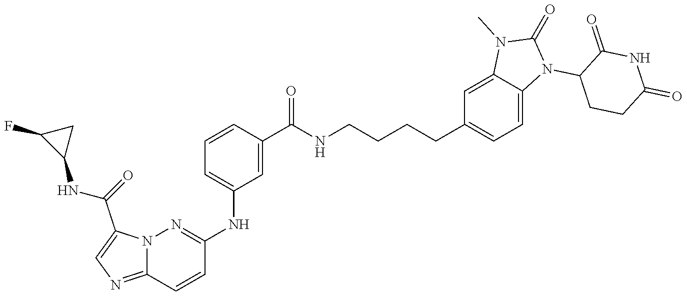 [SMILES: Cn1c(=O)n(C2CCC(=O)NC2=O)c2ccc(CCCCNC(=O)c3cccc(Nc4ccc5ncc(C(=O)N[C@@H]6C[C@@H]6F)n5n4)c3)cc21] |
| K329 | 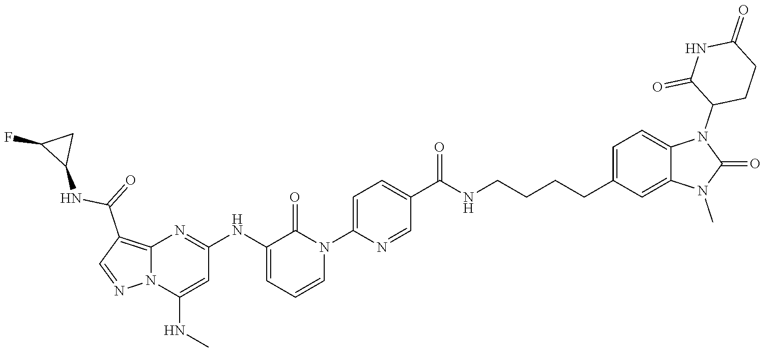 [SMILES: CNc1cc(Nc2cccn(-c3ccc(C(=O)NCCCCc4ccc5c(c4)n(C)c(=O)n5C4CCC(=O)NC4=O)cn3)c2=O)nc2c(C(=O)N[C@@H]3C[C@@H]3F)cnn12] |
| K330 | 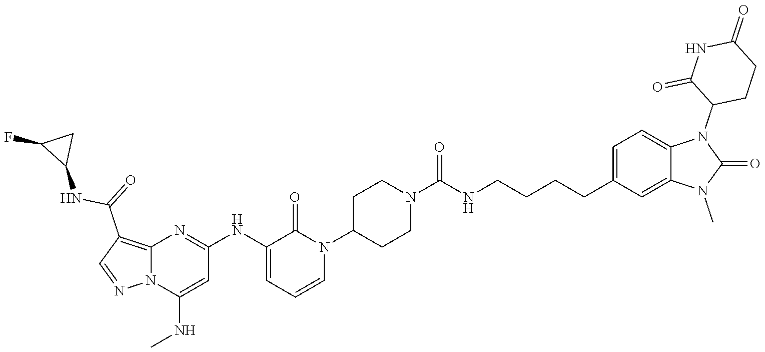 [SMILES: CNc1cc(Nc2cccn(C3CCN(C(=O)NCCCCc4ccc5c(c4)n(C)c(=O)n5C4CCC(=O)NC4=O)CC3)c2=O)nc2c(C(=O)N[C@@H]3C[C@@H]3F)cnn12] |
| K331 | 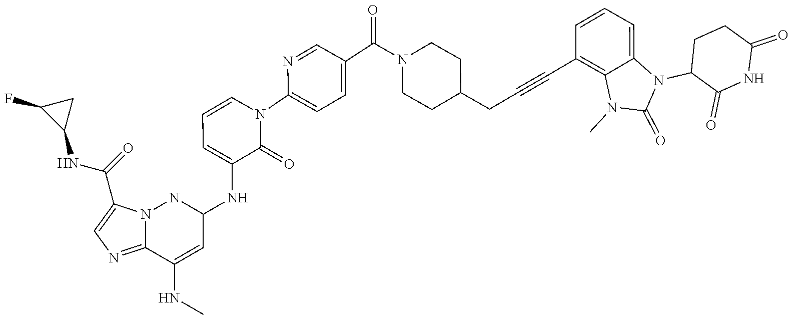 [SMILES: CNC1=CC(Nc2cccn(-c3ccc(C(=O)N4CCC(CC#Cc5cccc6c5n(C)c(=O)n6C5CCC(=O)NC5=O)CC4)cn3)c2=O)Nn2c(C(=O)N[C@@H]3C[C@@H]3F)cnc21] |
| K332 | 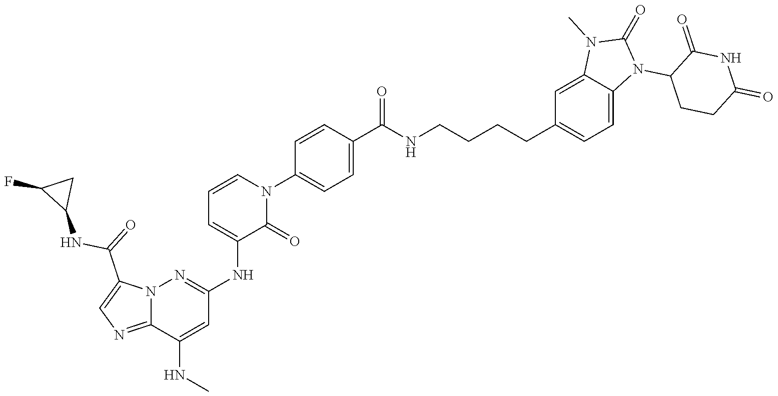 [SMILES: CNc1cc(Nc2cccn(-c3ccc(C(=O)NCCCCc4ccc5c(c4)n(C)c(=O)n5C4CCC(=O)NC4=O)cc3)c2=O)nn2c(C(=O)N[C@@H]3C[C@@H]3F)cnc12] |
| K333 | 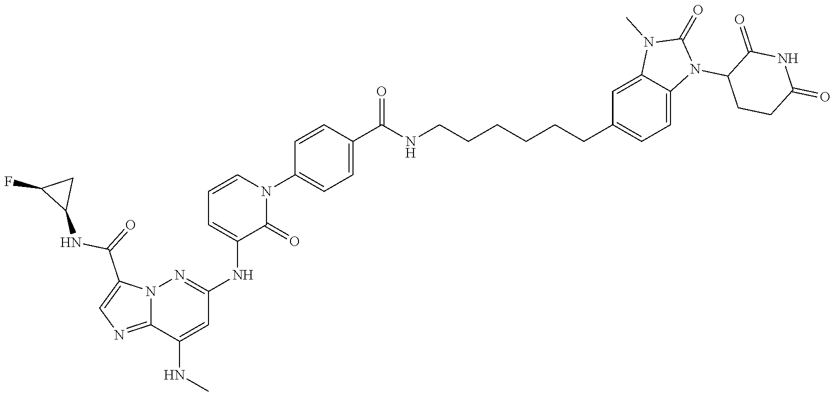 [SMILES: CNc1cc(Nc2cccn(-c3ccc(C(=O)NCCCCCCc4ccc5c(c4)n(C)c(=O)n5C4CCC(=O)NC4=O)cc3)c2=O)nn2c(C(=O)N[C@@H]3C[C@@H]3F)cnc12] |
| K334 | 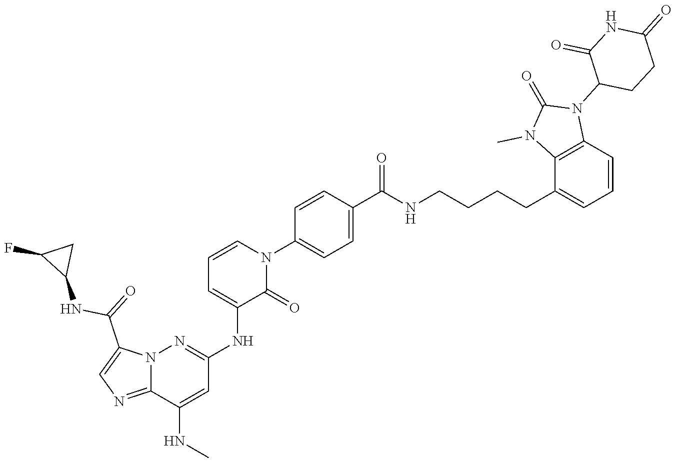 [SMILES: CNc1cc(Nc2cccn(-c3ccc(C(=O)NCCCCc4cccc5c4n(C)c(=O)n5C4CCC(=O)NC4=O)cc3)c2=O)nn2c(C(=O)N[C@@H]3C[C@@H]3F)cnc12] |
| K335 | 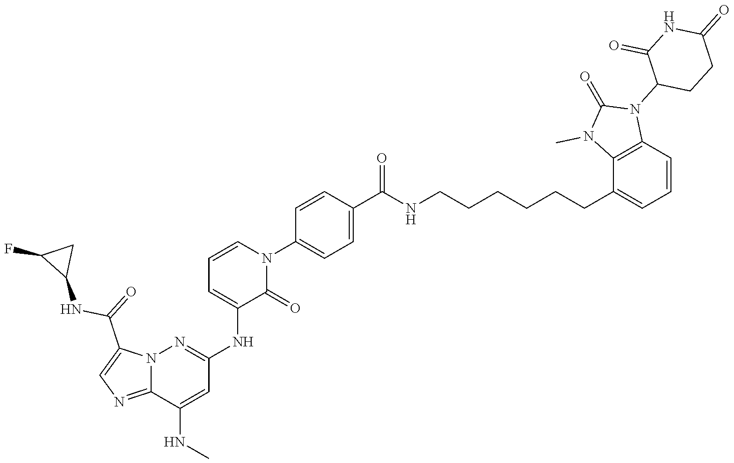 [SMILES: CNc1cc(Nc2cccn(-c3ccc(C(=O)NCCCCCCc4cccc5c4n(C)c(=O)n5C4CCC(=O)NC4=O)cc3)c2=O)nn2c(C(=O)N[C@@H]3C[C@@H]3F)cnc12] |
| K336 | 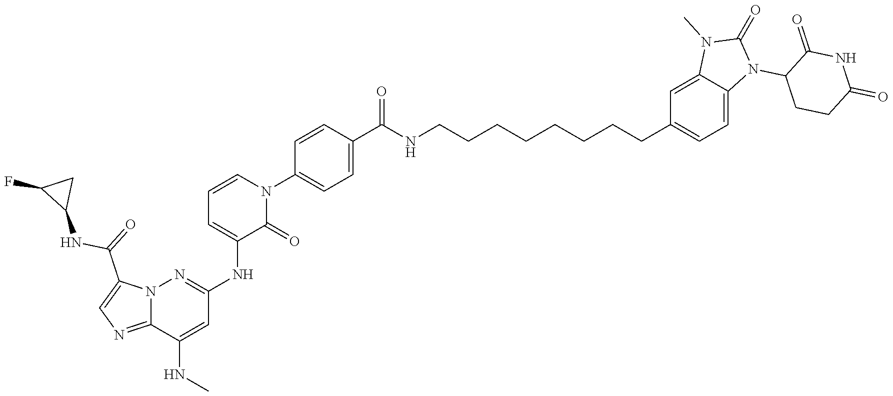 [SMILES: CNc1cc(Nc2cccn(-c3ccc(C(=O)NCCCCCCCCc4ccc5c(c4)n(C)c(=O)n5C4CCC(=O)NC4=O)cc3)c2=O)nn2c(C(=O)N[C@@H]3C[C@@H]3F)cnc12] |
| K337 | 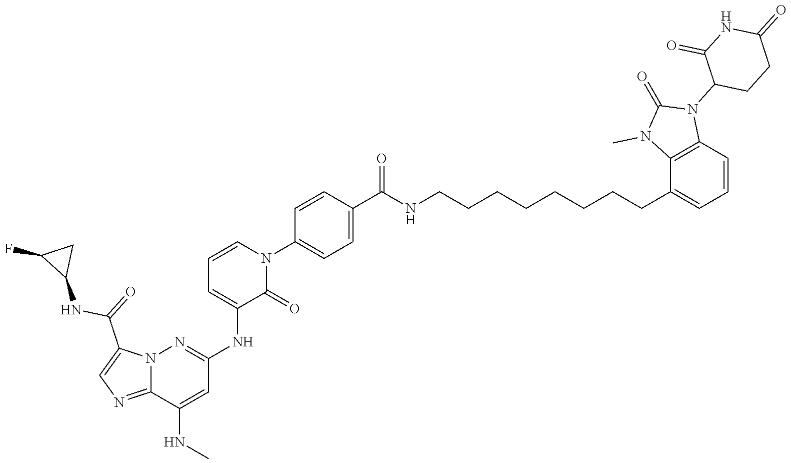 [SMILES: CNc1cc(Nc2cccn(-c3ccc(C(=O)NCCCCCCCCc4cccc5c4n(C)c(=O)n5C4CCC(=O)NC4=O)cc3)c2=O)nn2c(C(=O)N[C@@H]3C[C@@H]3F)cnc12] |
| K338 | 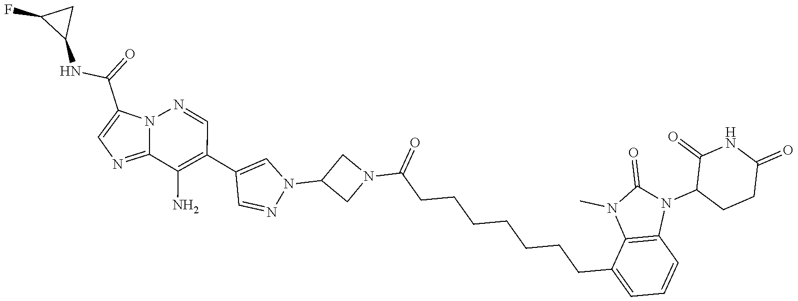 [SMILES: Cn1c(=O)n(C2CCC(=O)NC2=O)c2cccc(CCCCCCCC(=O)N3CC(n4cc(-c5cnn6c(C(=O)N[C@@H]7C[C@@H]7F)cnc6c5N)cn4)C3)c21] |
| K339 | 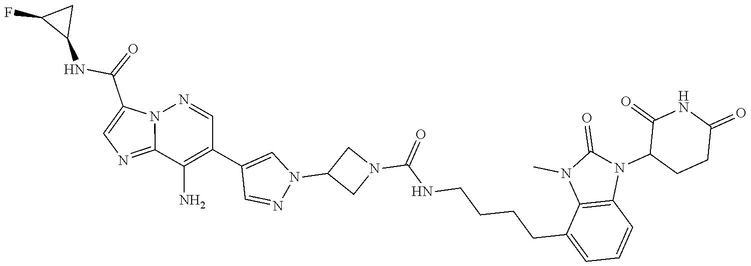 [SMILES: Cn1c(=O)n(C2CCC(=O)NC2=O)c2cccc(CCCCNC(=O)N3CC(n4cc(-c5cnn6c(C(=O)N[C@@H]7C[C@@H]7F)cnc6c5N)cn4)C3)c21] |
| K340 | 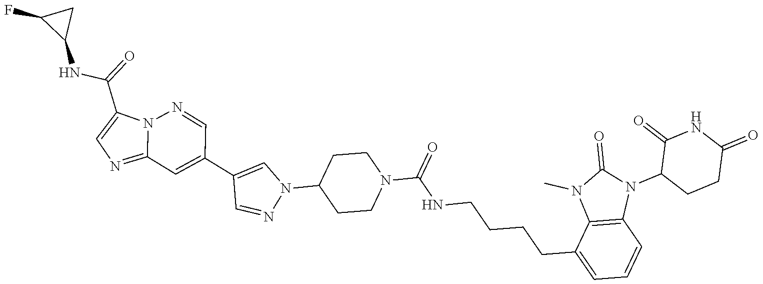 [SMILES: Cn1c(=O)n(C2CCC(=O)NC2=O)c2cccc(CCCCNC(=O)N3CCC(n4cc(-c5cnn6c(C(=O)N[C@@H]7C[C@@H]7F)cnc6c5)cn4)CC3)c21] |
| K341 | 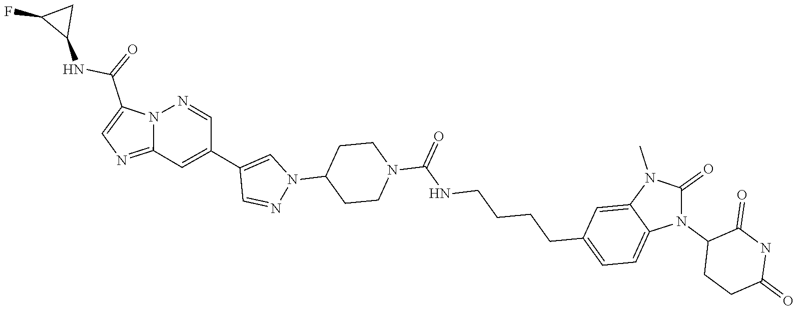 |
| K342 | 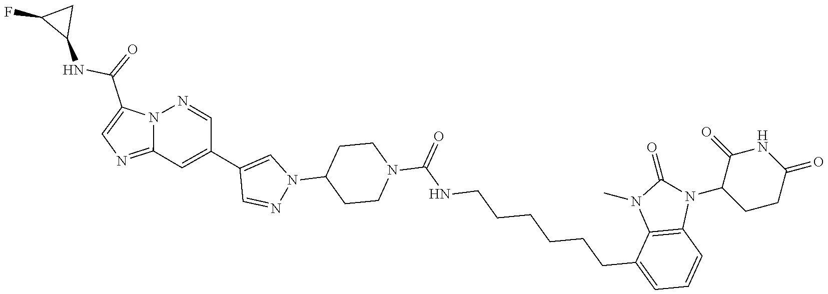 [SMILES: Cn1c(=O)n(C2CCC(=O)NC2=O)c2cccc(CCCCCCNC(=O)N3CCC(n4cc(-c5cnn6c(C(=O)N[C@@H]7C[C@@H]7F)cnc6c5)cn4)CC3)c21] |
| K343 | 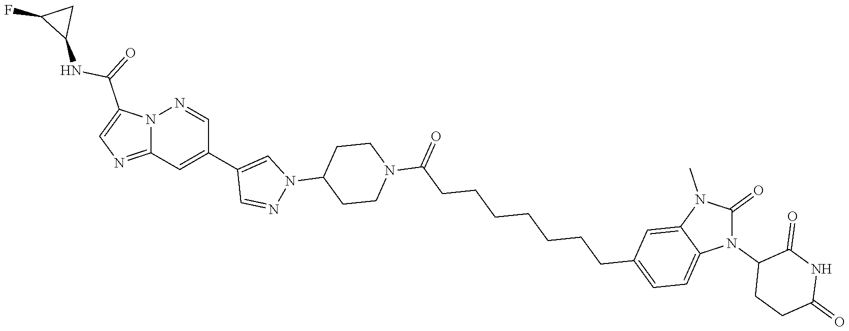 [SMILES: Cn1c(=O)n(C2CCC(=O)NC2=O)c2ccc(CCCCCCCC(=O)N3CCC(n4cc(-c5cnn6c(C(=O)N[C@@H]7C[C@@H]7F)cnc6c5)cn4)CC3)cc21] |
| K344 | 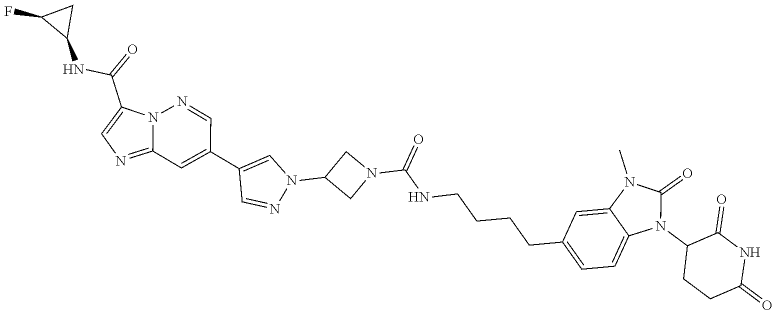 [SMILES: Cn1c(=O)n(C2CCC(=O)NC2=O)c2ccc(CCCCNC(=O)N3CC(n4cc(-c5cnn6c(C(=O)N[C@@H]7C[C@@H]7F)cnc6c5)cn4)C3)cc21] |
| K345 | 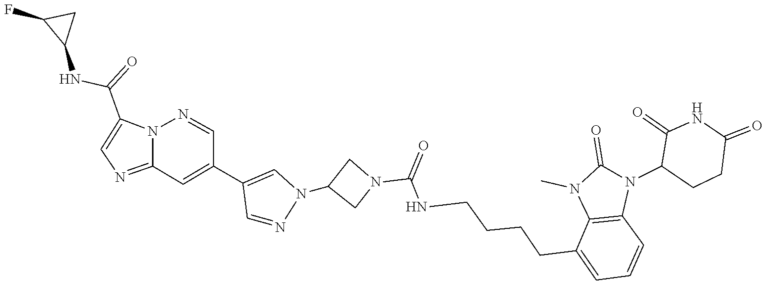 [SMILES: Cn1c(=O)n(C2CCC(=O)NC2=O)c2cccc(CCCCNC(=O)N3CC(n4cc(-c5cnn6c(C(=O)N[C@@H]7C[C@@H]7F)cnc6c5)cn4)C3)c21] |
| K346 | 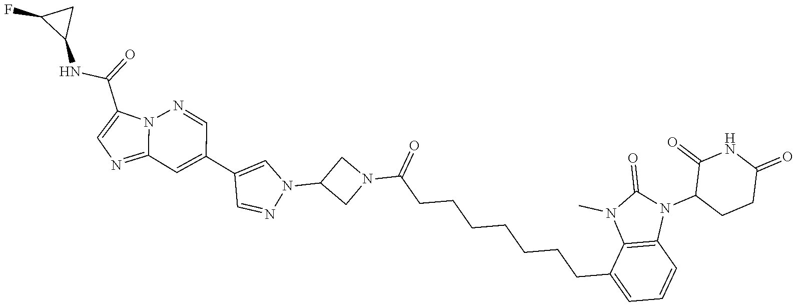 [SMILES: Cn1c(=O)n(C2CCC(=O)NC2=O)c2cccc(CCCCCCCC(=O)N3CC(n4cc(-c5cnn6c(C(=O)N[C@@H]7C[C@@H]7F)cnc6c5)cn4)C3)c21] |
| K347 | 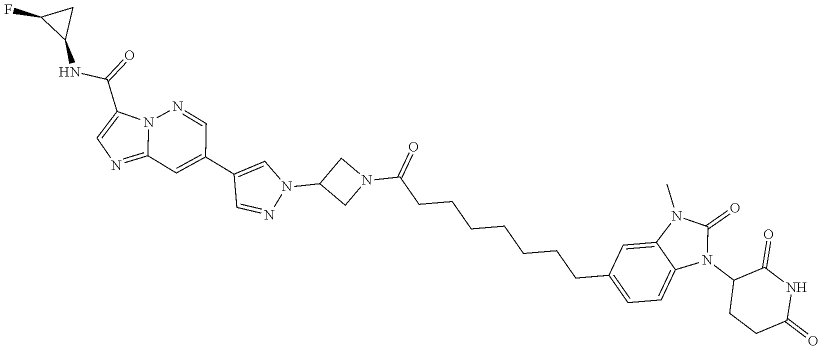 [SMILES: Cn1c(=O)n(C2CCC(=O)NC2=O)c2ccc(CCCCCCCC(=O)N3CC(n4cc(-c5cnn6c(C(=O)N[C@@H]7C[C@@H]7F)cnc6c5)cn4)C3)cc21] |
| K348 | 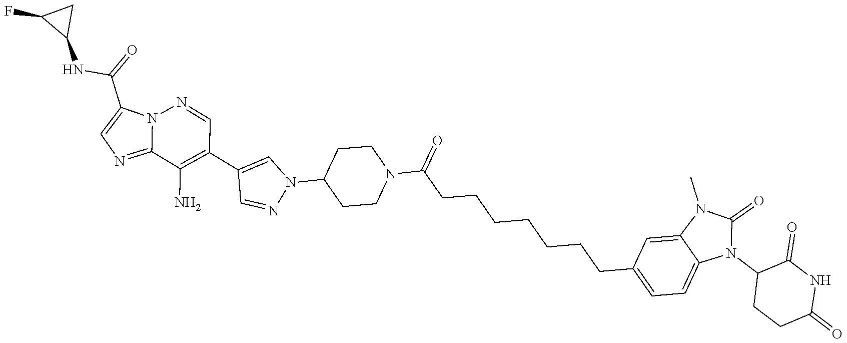 [SMILES: Cn1c(=O)n(C2CCC(=O)NC2=O)c2ccc(CCCCCCCC(=O)N3CCC(n4cc(-c5cnn6c(C(=O)N[C@@H]7C[C@@H]7F)cnc6c5N)cn4)CC3)cc21] |
| K349 | 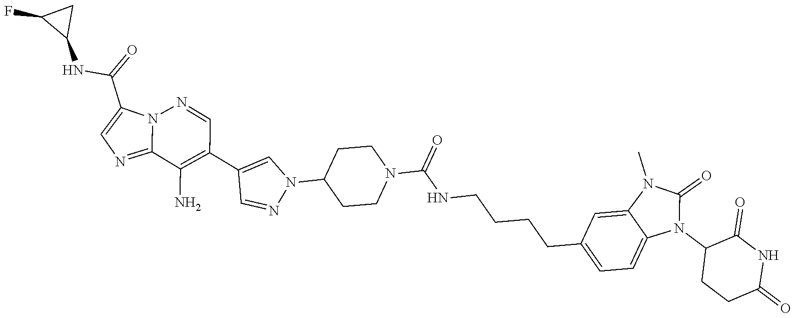 [SMILES: Cn1c(=O)n(C2CCC(=O)NC2=O)c2ccc(CCCCNC(=O)N3CCC(n4cc(-c5cnn6c(C(=O)N[C@@H]7C[C@@H]7F)cnc6c5N)cn4)CC3)cc21] |
| K350 | 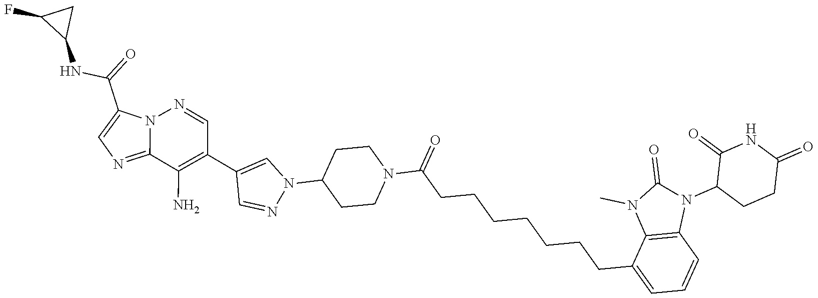 [SMILES: Cn1c(=O)n(C2CCC(=O)NC2=O)c2cccc(CCCCCCCC(=O)N3CCC(n4cc(-c5cnn6c(C(=O)N[C@@H]7C[C@@H]7F)cnc6c5N)cn4)CC3)c21] |
| K351 | 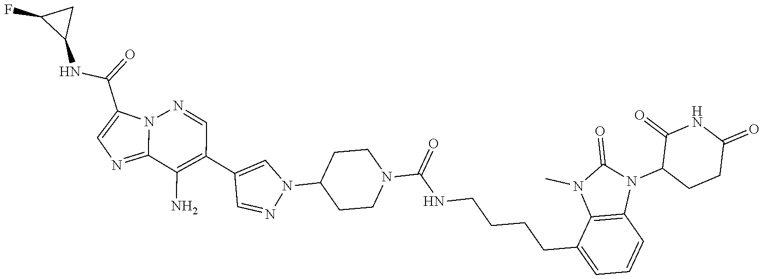 [SMILES: Cn1c(=O)n(C2CCC(=O)NC2=O)c2cccc(CCCCNC(=O)N3CCC(n4cc(-c5cnn6c(C(=O)N[C@@H]7C[C@@H]7F)cnc6c5N)cn4)CC3)c21] |
| K352 | 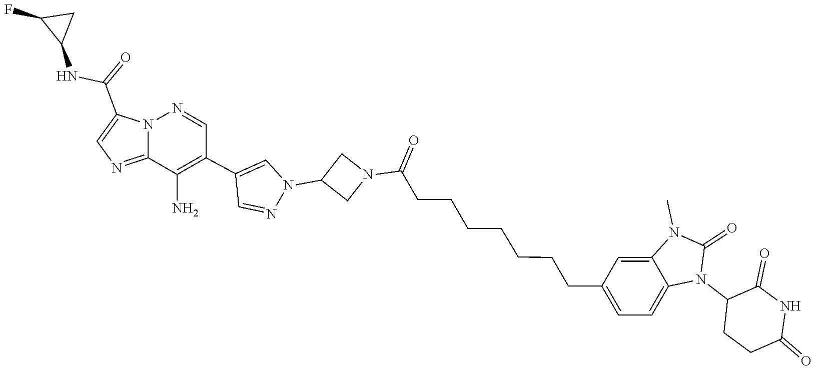 [SMILES: Cn1c(=O)n(C2CCC(=O)NC2=O)c2ccc(CCCCCCCC(=O)N3CC(n4cc(-c5cnn6c(C(=O)N[C@@H]7C[C@@H]7F)cnc6c5N)cn4)C3)cc21] |
| K353 | 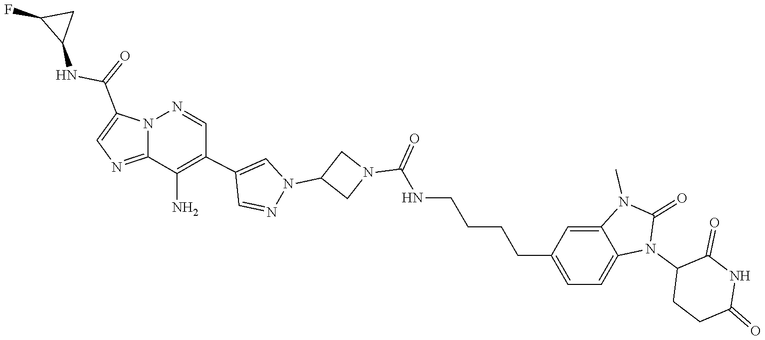 [SMILES: Cn1c(=O)n(C2CCC(=O)NC2=O)c2ccc(CCCCNC(=O)N3CC(n4cc(-c5cnn6c(C(=O)N[C@@H]7C[C@@H]7F)cnc6c5N)cn4)C3)cc21] |
| K354 | 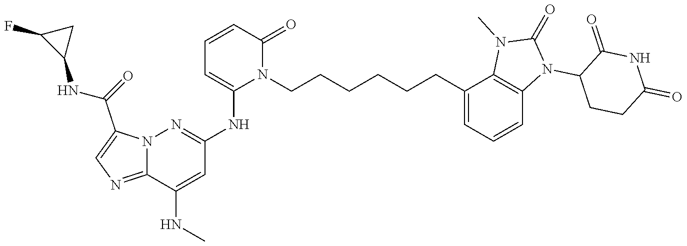 [SMILES: CNc1cc(Nc2cccc(=O)n2CCCCCCc2cccc3c2n(C)c(=O)n3C2CCC(=O)NC2=O)nn2c(C(=O)N[C@@H]3C[C@@H]3F)cnc12] |
| K355 | 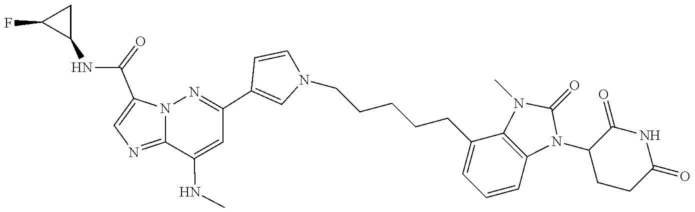 [SMILES: CNc1cc(-c2ccn(CCCCCc3cccc4c3n(C)c(=O)n4C3CCC(=O)NC3=O)c2)nn2c(C(=O)N[C@@H]3C[C@@H]3F)cnc12] |
| K356 | 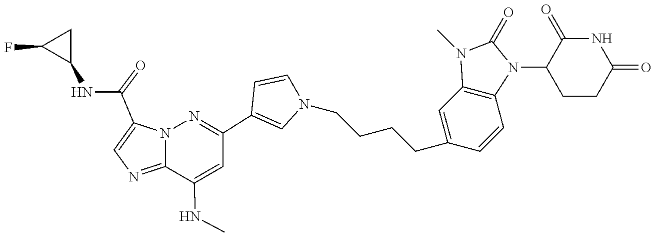 [SMILES: CNc1cc(-c2ccn(CCCCc3ccc4c(c3)n(C)c(=O)n4C3CCC(=O)NC3=O)c2)nn2c(C(=O)N[C@@H]3C[C@@H]3F)cnc12] |
| K357 | 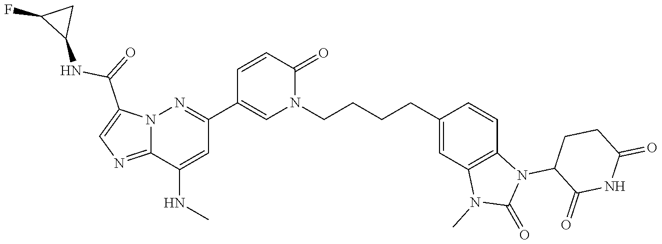 [SMILES: CNc1cc(-c2ccc(=O)n(CCCCc3ccc4c(c3)n(C)c(=O)n4C3CCC(=O)NC3=O)c2)nn2c(C(=O)N[C@@H]3C[C@@H]3F)cnc12] |
| K358 | 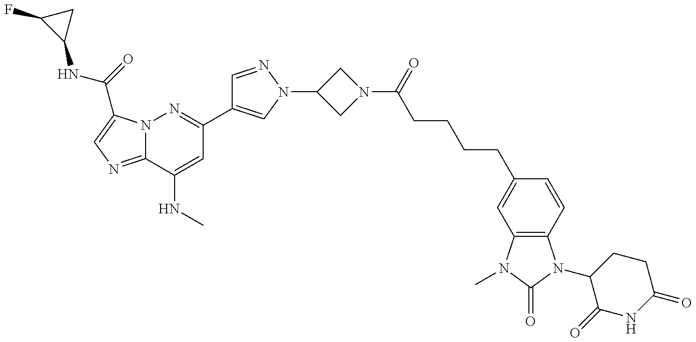 [SMILES: CNc1cc(-c2cnn(C3CN(C(=O)CCCCc4ccc5c(c4)n(C)c(=O)n5C4CCC(=O)NC4=O)C3)c2)nn2c(C(=O)N[C@@H]3C[C@@H]3F)cnc12] |
| K359 | 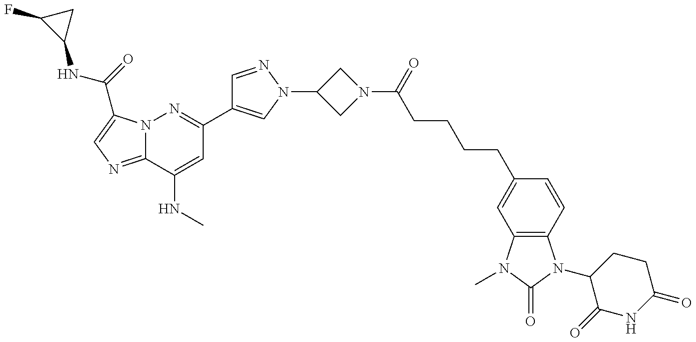 [SMILES: CNc1cc(-c2cnn(C3CN(C(=O)CCCCc4ccc5c(c4)n(C)c(=O)n5C4CCC(=O)NC4=O)C3)c2)nn2c(C(=O)N[C@@H]3C[C@@H]3F)cnc12] |
| K360 | 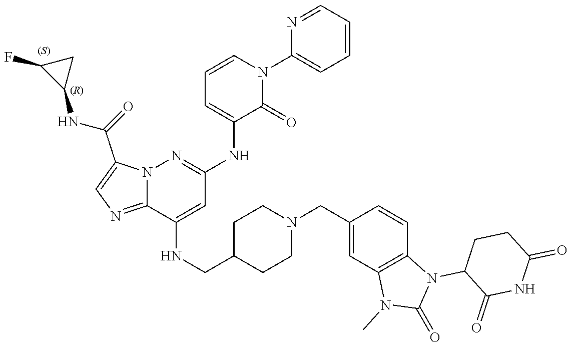 |
| K361 | 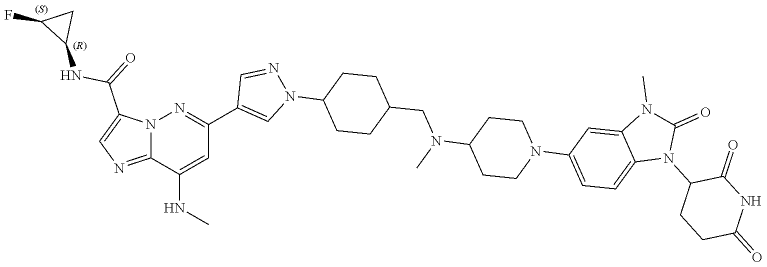 |
| K362 | 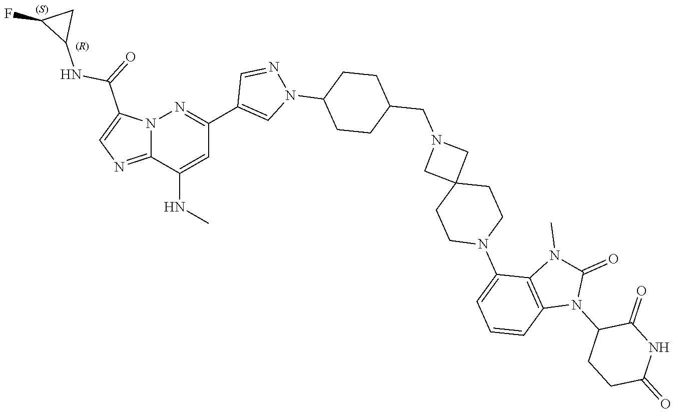 |
| K363 | 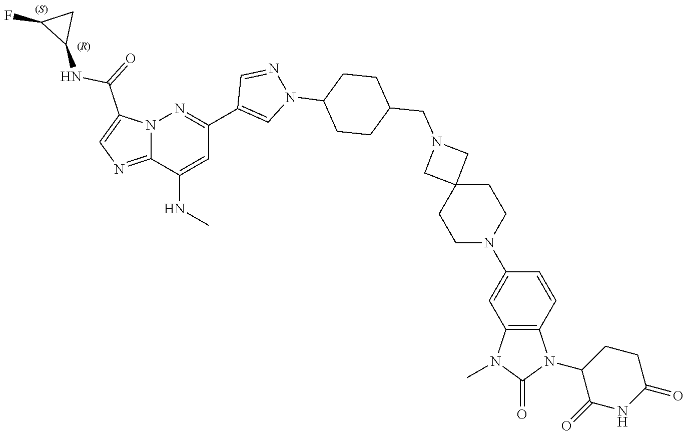 |
| K364 | 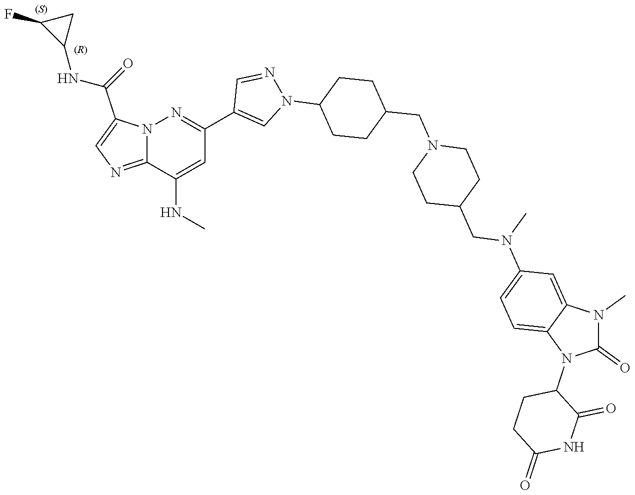 |
| K365 | 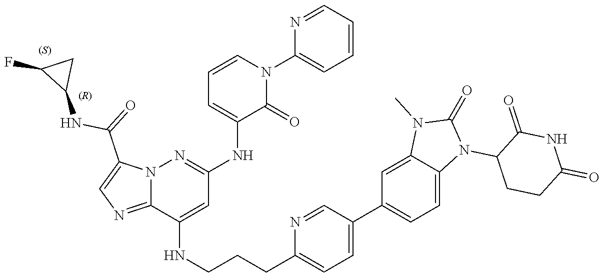 |
| K366 | 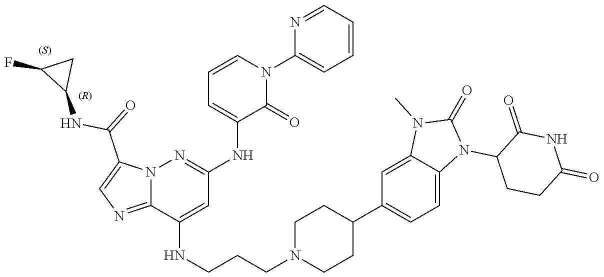 |
| K367 | 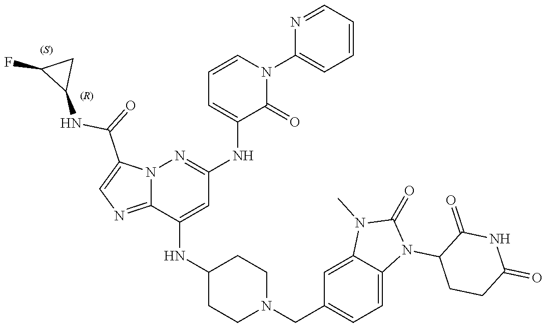 |
| K368 | 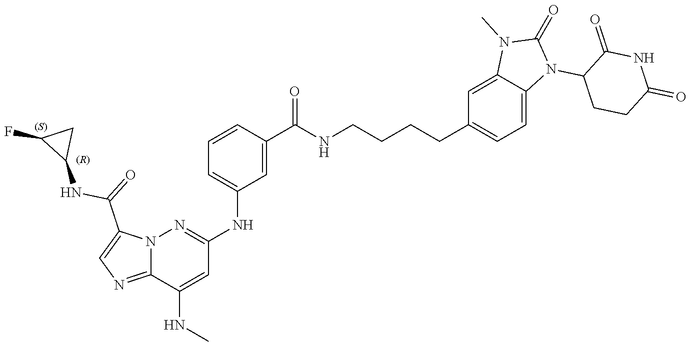 |
| K369 | 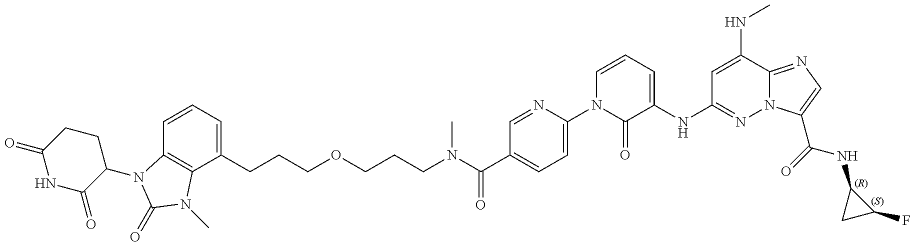 |
| K370 | 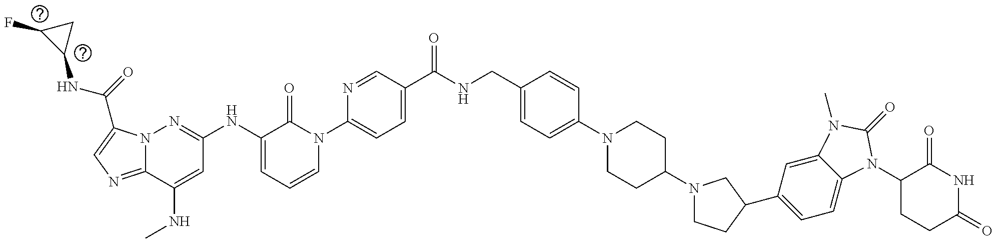 [SMILES: CNc1cc(Nc2cccn(-c3ccc(C(=O)NCc4ccc(N5CCC(N6CCC(c7ccc8c(c7)n(C)c(=O)n8C7CCC(=O)NC7=O)C6)CC5)cc4)cn3)c2=O)nn2c(C(=O)N[C@@H]3C[C@@H]3F)cnc12] |
| K371 | 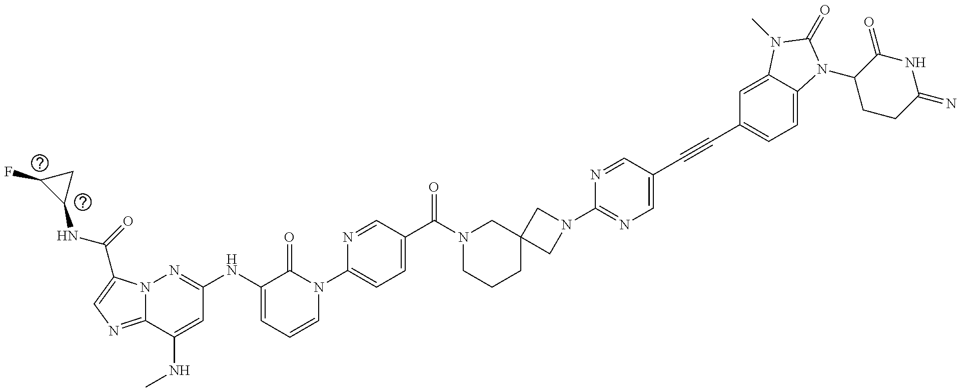 [SMILES: CNc1cc(Nc2cccn(-c3ccc(C(=O)N4CCCC5(C4)CN(c4ncc(C#Cc6ccc7c(c6)n(C)c(=O)n7C6CCC(=N)NC6=O)cn4)C5)cn3)c2=O)nn2c(C(=O)N[C@@H]3C[C@@H]3F)cnc12] |
| K372 | 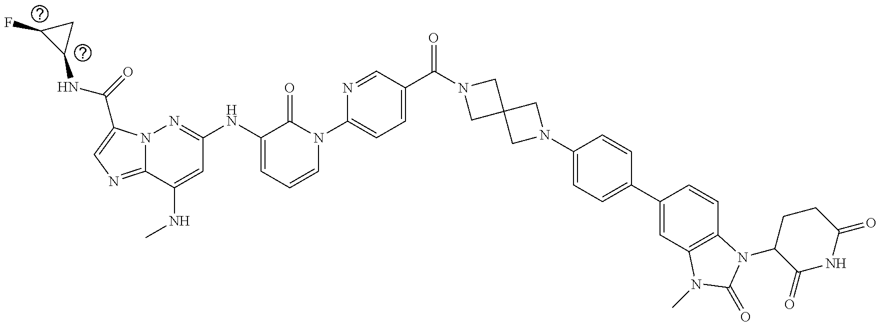 [SMILES: CNc1cc(Nc2cccn(-c3ccc(C(=O)N4CC5(C4)CN(c4ccc(-c6ccc7c(c6)n(C)c(=O)n7C6CCC(=O)NC6=O)cc4)C5)cn3)c2=O)nn2c(C(=O)N[C@@H]3C[C@@H]3F)cnc12] |
| K373 | 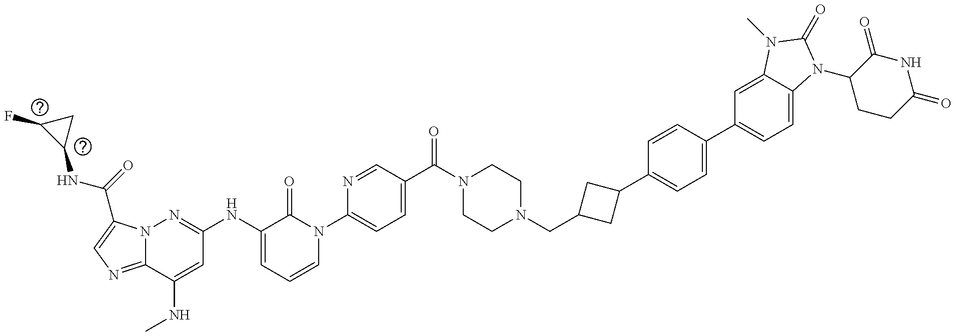 [SMILES: CNc1cc(Nc2cccn(-c3ccc(C(=O)N4CCN(CC5CC(c6ccc(-c7ccc8c(c7)n(C)c(=O)n8C7CCC(=O)NC7=O)cc6)C5)CC4)cn3)c2=O)nn2c(C(=O)N[C@@H]3C[C@@H]3F)cnc12] |
| K374 | 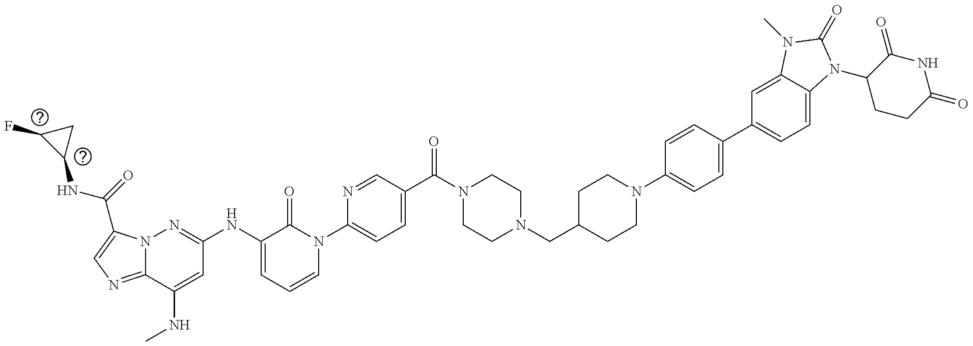 [SMILES: CNc1cc(Nc2cccn(-c3ccc(C(=O)N4CCN(CC5CCN(c6ccc(-c7ccc8c(c7)n(C)c(=O)n8C7CCC(=O)NC7=O)cc6)CC5)CC4)cn3)c2=O)nn2c(C(=O)N[C@@H]3C[C@@H]3F)cnc12] |
| K375 | 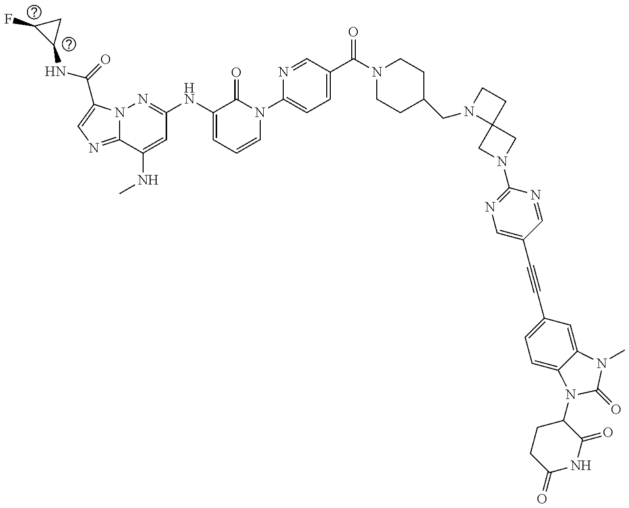 [SMILES: CNc1cc(Nc2cccn(-c3ccc(C(=O)N4CCC(CN5CCC56CN(c5ncc(C#Cc7ccc8c(c7)n(C)c(=O)n8C7CCC(=O)NC7=O)cn5)C6)CC4)cn3)c2=O)nn2c(C(=O)N[C@@H]3C[C@@H]3F)cnc12] |
| K376 | 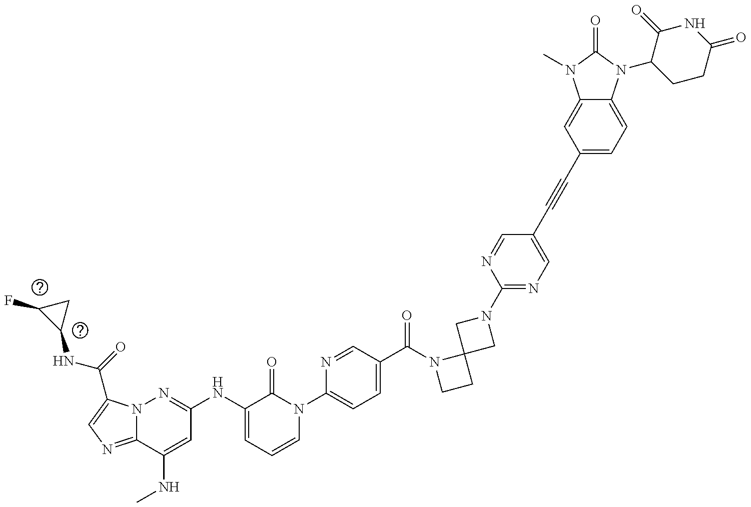 [SMILES: CNc1cc(Nc2cccn(-c3ccc(C(=O)N4CCC45CN(c4ncc(C#Cc6ccc7c(c6)n(C)c(=O)n7C6CCC(=O)NC6=O)cn4)C5)cn3)c2=O)nn2c(C(=O)N[C@@H]3C[C@@H]3F)cnc12] |
| K377 | 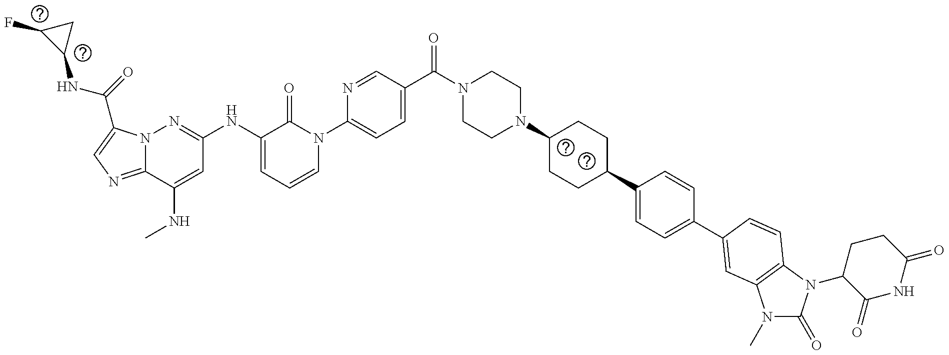 [SMILES: CNc1cc(Nc2cccn(-c3ccc(C(=O)N4CCN([C@H]5CC[C@@H](c6ccc(-c7ccc8c(c7)n(C)c(=O)n8C7CCC(=O)NC7=O)cc6)CC5)CC4)cn3)c2=O)nn2c(C(=O)N[C@@H]3C[C@@H]3F)cnc12] |
| K378 | 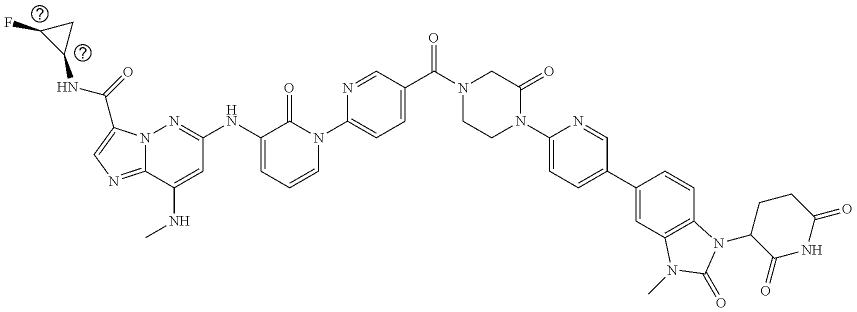 [SMILES: CNc1cc(Nc2cccn(-c3ccc(C(=O)N4CCN(c5ccc(-c6ccc7c(c6)n(C)c(=O)n7C6CCC(=O)NC6=O)cn5)C(=O)C4)cn3)c2=O)nn2c(C(=O)N[C@@H]3C[C@@H]3F)cnc12] |
| K379 | 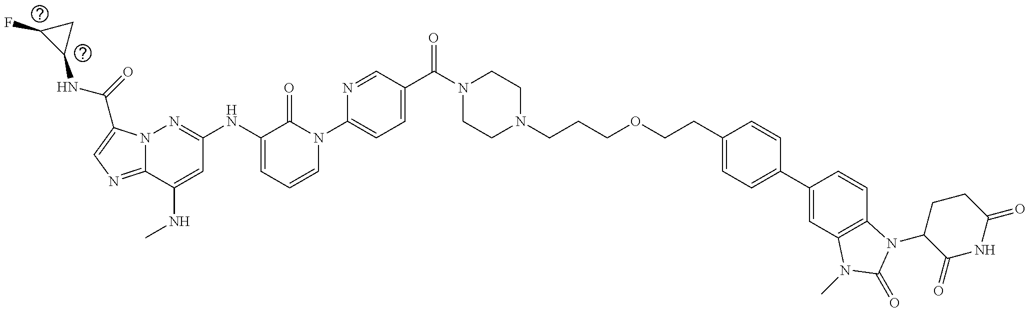 [SMILES: CNc1cc(Nc2cccn(-c3ccc(C(=O)N4CCN(CCCOCCc5ccc(-c6ccc7c(c6)n(C)c(=O)n7C6CCC(=O)NC6=O)cc5)CC4)cn3)c2=O)nn2c(C(=O)N[C@@H]3C[C@@H]3F)cnc12] |
| K380 | 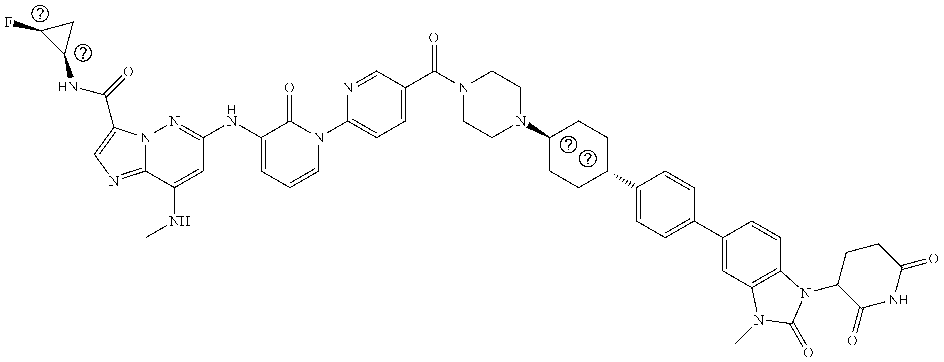 [SMILES: CNc1cc(Nc2cccn(-c3ccc(C(=O)N4CCN([C@H]5CC[C@H](c6ccc(-c7ccc8c(c7)n(C)c(=O)n8C7CCC(=O)NC7=O)cc6)CC5)CC4)cn3)c2=O)nn2c(C(=O)N[C@@H]3C[C@@H]3F)cnc12] |
| K381 | 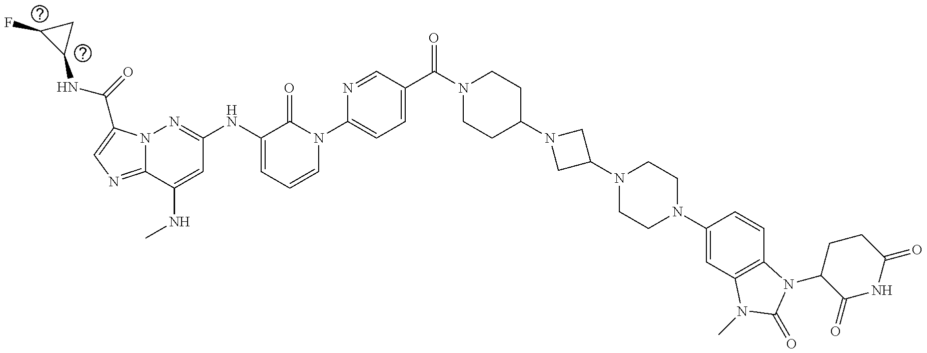 [SMILES: CNc1cc(Nc2cccn(-c3ccc(C(=O)N4CCC(N5CC(N6CCN(c7ccc8c(c7)n(C)c(=O)n8C7CCC(=O)NC7=O)CC6)C5)CC4)cn3)c2=O)nn2c(C(=O)N[C@@H]3C[C@@H]3F)cnc12] |
| K382 | 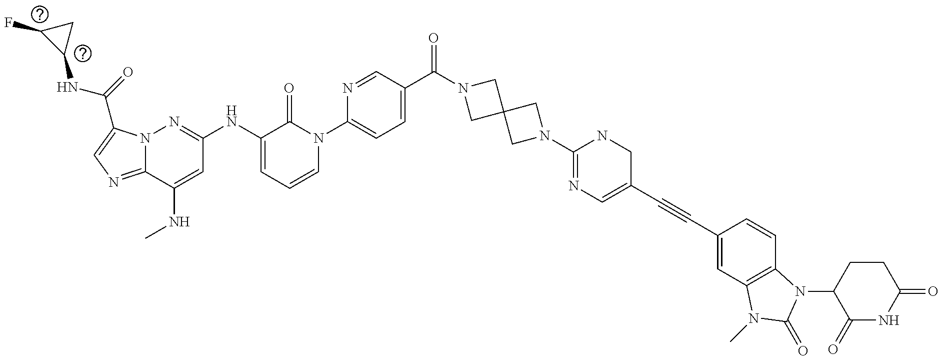 [SMILES: CNc1cc(Nc2cccn(-c3ccc(C(=O)N4CC5(C4)CN(C4=NC=C(C#Cc6ccc7c(c6)n(C)c(=O)n7C6CCC(=O)NC6=O)CN4)C5)cn3)c2=O)nn2c(C(=O)N[C@@H]3C[C@@H]3F)cnc12] |
| K383 | 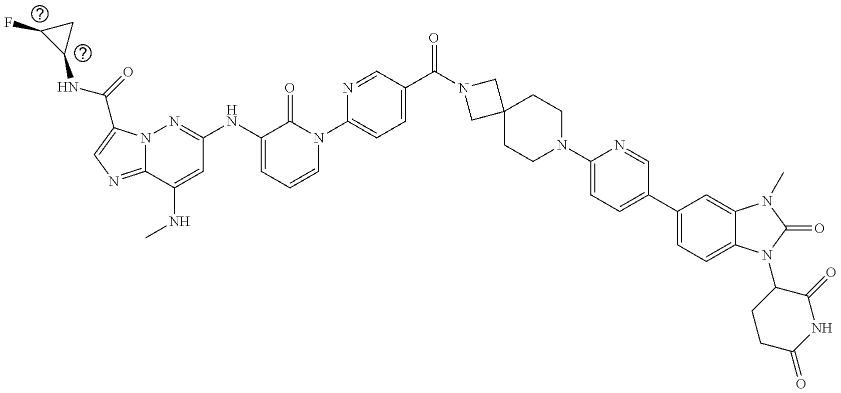 [SMILES: CNc1cc(Nc2cccn(-c3ccc(C(=O)N4CC5(CCN(c6ccc(-c7ccc8c(c7)n(C)c(=O)n8C7CCC(=O)NC7=O)cn6)CC5)C4)cn3)c2=O)nn2c(C(=O)N[C@@H]3C[C@@H]3F)cnc12] |
| K384 | 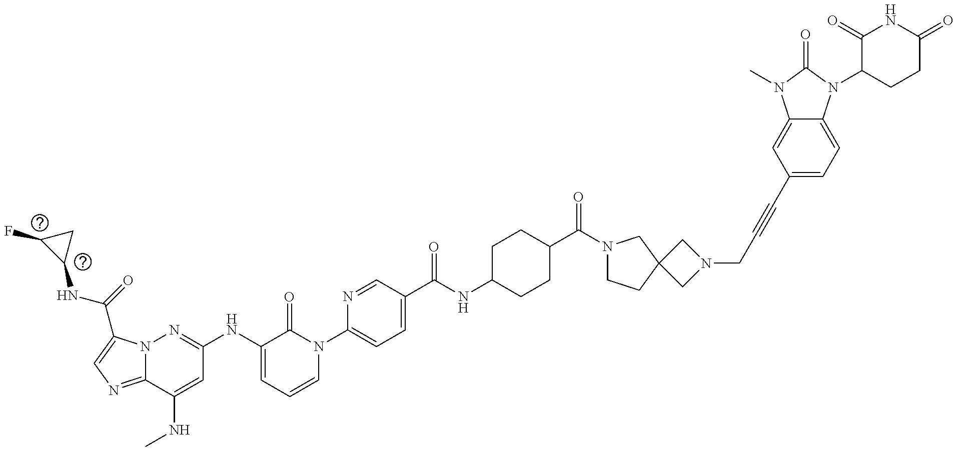 [SMILES: CNc1cc(Nc2cccn(-c3ccc(C(=O)NC4CCC(C(=O)N5CCC6(CN(CC#Cc7ccc8c(c7)n(C)c(=O)n8C7CCC(=O)NC7=O)C6)C5)CC4)cn3)c2=O)nn2c(C(=O)N[C@@H]3C[C@@H]3F)cnc12] |
| K385 | 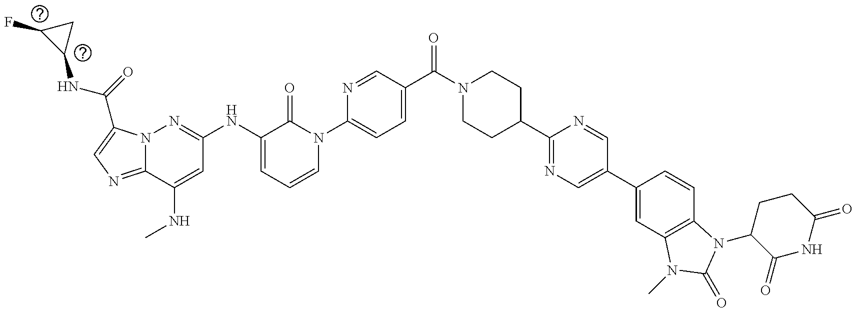 [SMILES: CNc1cc(Nc2cccn(-c3ccc(C(=O)N4CCC(c5ncc(-c6ccc7c(c6)n(C)c(=O)n7C6CCC(=O)NC6=O)cn5)CC4)cn3)c2=O)nn2c(C(=O)N[C@@H]3C[C@@H]3F)cnc12] |
| K386 | 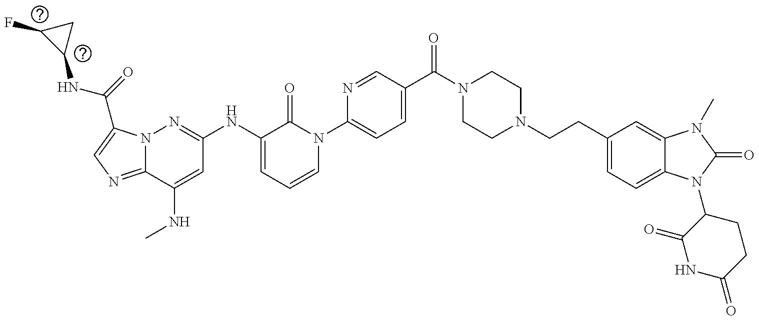 [SMILES: CNc1cc(Nc2cccn(-c3ccc(C(=O)N4CCN(CCc5ccc6c(c5)n(C)c(=O)n6C5CCC(=O)NC5=O)CC4)cn3)c2=O)nn2c(C(=O)N[C@@H]3C[C@@H]3F)cnc12] |
| K387 | 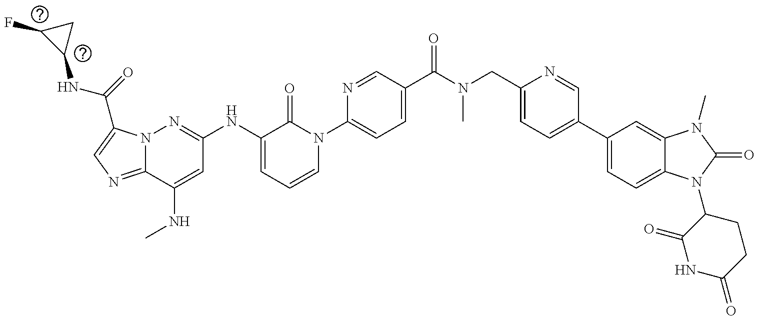 [SMILES: CNc1cc(Nc2cccn(-c3ccc(C(=O)N(C)Cc4ccc(-c5ccc6c(c5)n(C)c(=O)n6C5CCC(=O)NC5=O)cn4)cn3)c2=O)nn2c(C(=O)N[C@@H]3C[C@@H]3F)cnc12] |
| K388 | 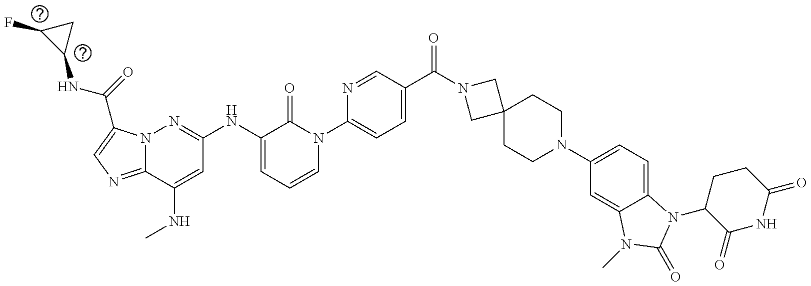 [SMILES: CNc1cc(Nc2cccn(-c3ccc(C(=O)N4CC5(CCN(c6ccc7c(c6)n(C)c(=O)n7C6CCC(=O)NC6=O)CC5)C4)cn3)c2=O)nn2c(C(=O)N[C@@H]3C[C@@H]3F)cnc12] |
| K389 | 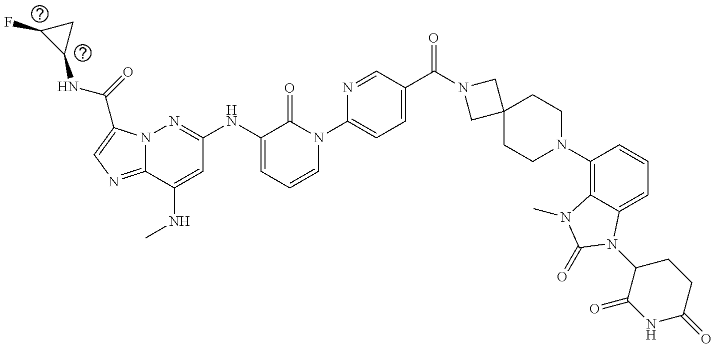 [SMILES: CNc1cc(Nc2cccn(-c3ccc(C(=O)N4CC5(CCN(c6cccc7c6n(C)c(=O)n7C6CCC(=O)NC6=O)CC5)C4)cn3)c2=O)nn2c(C(=O)N[C@@H]3C[C@@H]3F)cnc12] |
| K390 | 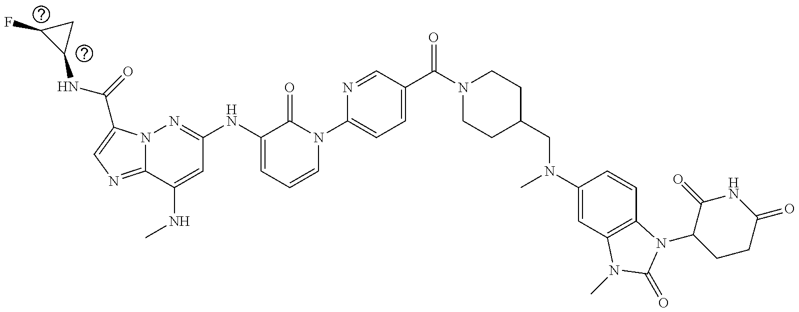 [SMILES: CNc1cc(Nc2cccn(-c3ccc(C(=O)N4CCC(CN(C)c5ccc6c(c5)n(C)c(=O)n6C5CCC(=O)NC5=O)CC4)cn3)c2=O)nn2c(C(=O)N[C@@H]3C[C@@H]3F)cnc12] |
| K391 | 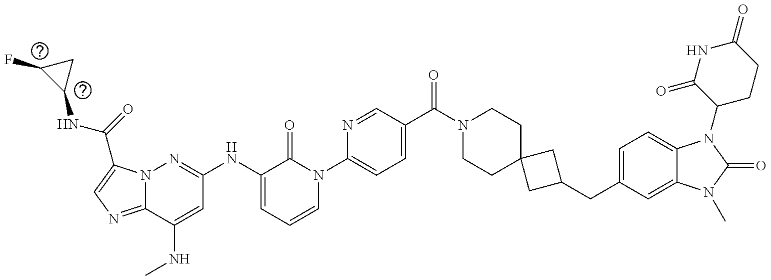 [SMILES: CNc1cc(Nc2cccn(-c3ccc(C(=O)N4CCC5(CC4)CC(Cc4ccc6c(c4)n(C)c(=O)n6C4CCC(=O)NC4=O)C5)cn3)c2=O)nn2c(C(=O)N[C@@H]3C[C@@H]3F)cnc12] |
| K392 | 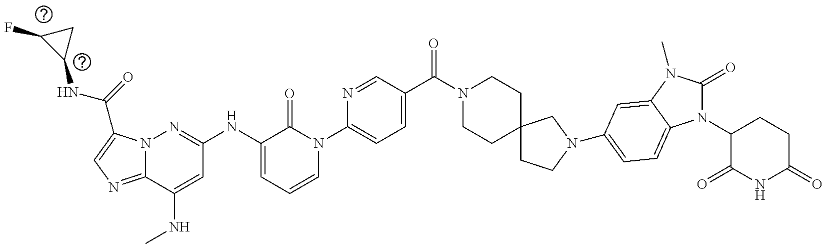 [SMILES: CNc1cc(Nc2cccn(-c3ccc(C(=O)N4CCC5(CC4)CCN(c4ccc6c(c4)n(C)c(=O)n6C4CCC(=O)NC4=O)C5)cn3)c2=O)nn2c(C(=O)N[C@@H]3C[C@@H]3F)cnc12] |
| K393 | 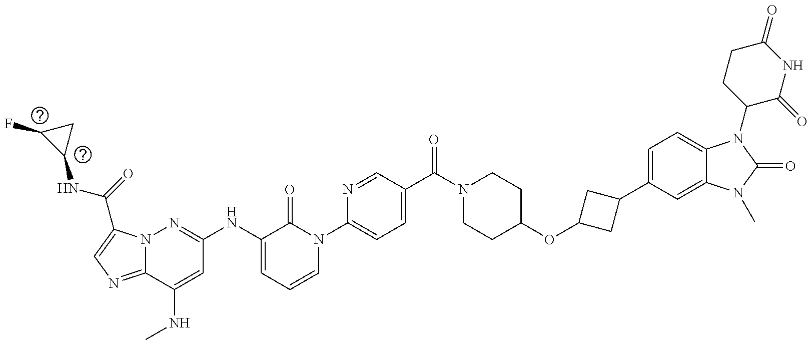 [SMILES: CNc1cc(Nc2cccn(-c3ccc(C(=O)N4CCC(OC5CC(c6ccc7c(c6)n(C)c(=O)n7C6CCC(=O)NC6=O)C5)CC4)cn3)c2=O)nn2c(C(=O)N[C@@H]3C[C@@H]3F)cnc12] |
| K394 | 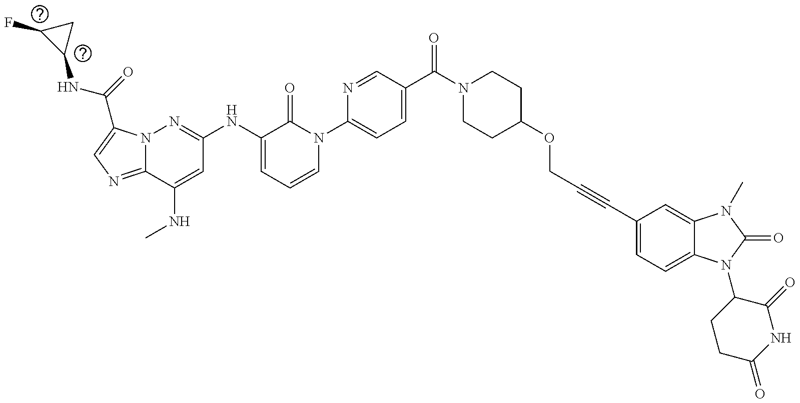 [SMILES: CNc1cc(Nc2cccn(-c3ccc(C(=O)N4CCC(OCC#Cc5ccc6c(c5)n(C)c(=O)n6C5CCC(=O)NC5=O)CC4)cn3)c2=O)nn2c(C(=O)N[C@@H]3C[C@@H]3F)cnc12] |
| K395 | 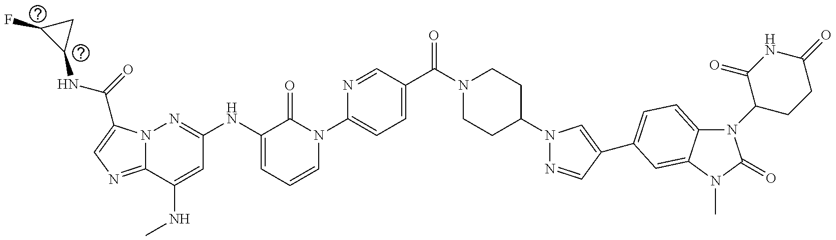 [SMILES: CNc1cc(Nc2cccn(-c3ccc(C(=O)N4CCC(n5cc(-c6ccc7c(c6)n(C)c(=O)n7C6CCC(=O)NC6=O)cn5)CC4)cn3)c2=O)nn2c(C(=O)N[C@@H]3C[C@@H]3F)cnc12] |
| K396 | 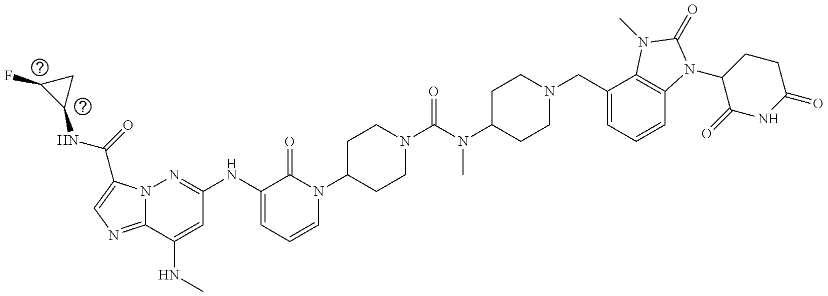 [SMILES: CNc1cc(Nc2cccn(C3CCN(C(=O)N(C)C4CCN(Cc5cccc6c5n(C)c(=O)n6C5CCC(=O)NC5=O)CC4)CC3)c2=O)nn2c(C(=O)N[C@@H]3C[C@@H]3F)cnc12] |
| K397 | 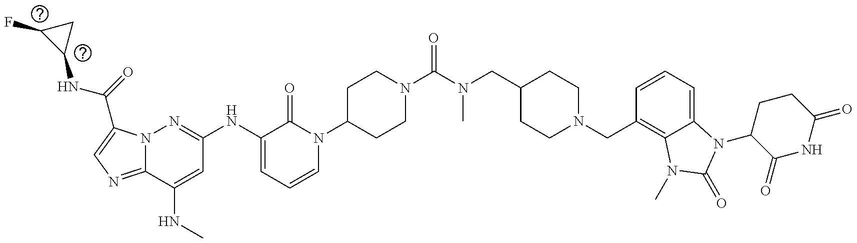 [SMILES: CNc1cc(Nc2cccn(C3CCN(C(=O)N(C)CC4CCN(Cc5cccc6c5n(C)c(=O)n6C5CCC(=O)NC5=O)CC4)CC3)c2=O)nn2c(C(=O)N[C@@H]3C[C@@H]3F)cnc12] |
| K398 | 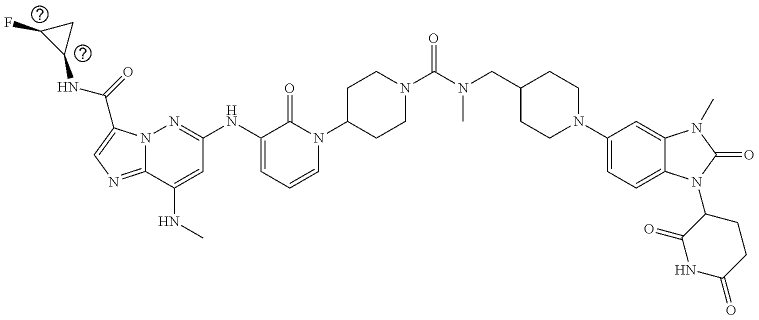 [SMILES: CNc1cc(Nc2cccn(C3CCN(C(=O)N(C)CC4CCN(c5ccc6c(c5)n(C)c(=O)n6C5CCC(=O)NC5=O)CC4)CC3)c2=O)nn2c(C(=O)N[C@@H]3C[C@@H]3F)cnc12] |
| K399 | 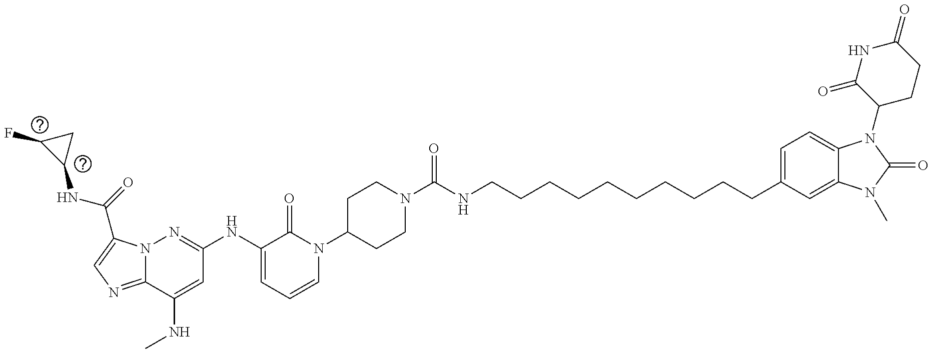 [SMILES: CNc1cc(Nc2cccn(C3CCN(C(=O)NCCCCCCCCCCc4ccc5c(c4)n(C)c(=O)n5C4CCC(=O)NC4=O)CC3)c2=O)nn2c(C(=O)N[C@@H]3C[C@@H]3F)cnc12] |
| K400 | 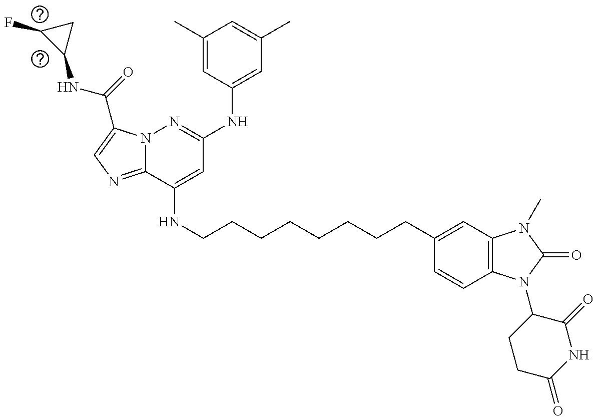 [SMILES: Cc1cc(C)cc(Nc2cc(NCCCCCCCCc3ccc4c(c3)n(C)c(=O)n4C3CCC(=O)NC3=O)c3ncc(C(=O)N[C@@H]4C[C@@H]4F)n3n2)c1] |
| K401 | 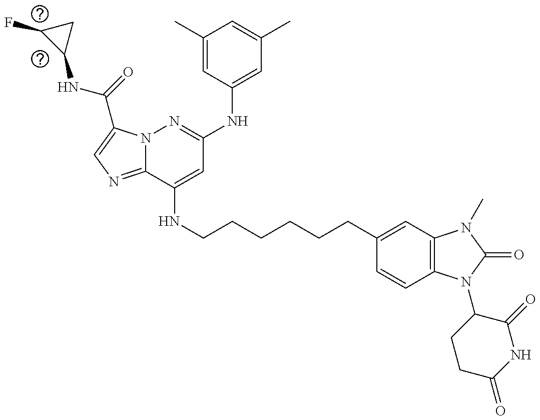 [SMILES: Cc1cc(C)cc(Nc2cc(NCCCCCCc3ccc4c(c3)n(C)c(=O)n4C3CCC(=O)NC3=O)c3ncc(C(=O)N[C@@H]4C[C@@H]4F)n3n2)c1] |
| K402 | 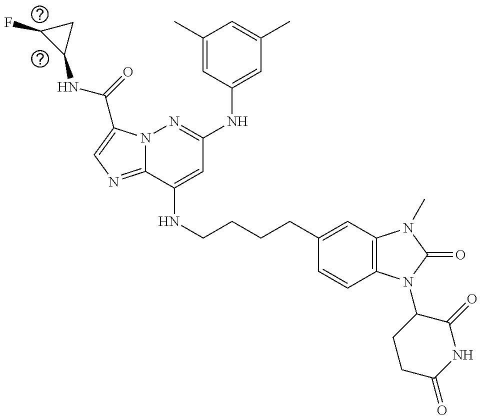 [SMILES: Cc1cc(C)cc(Nc2cc(NCCCCc3ccc4c(c3)n(C)c(=O)n4C3CCC(=O)NC3=O)c3ncc(C(=O)N[C@@H]4C[C@@H]4F)n3n2)c1] |
| K403 | 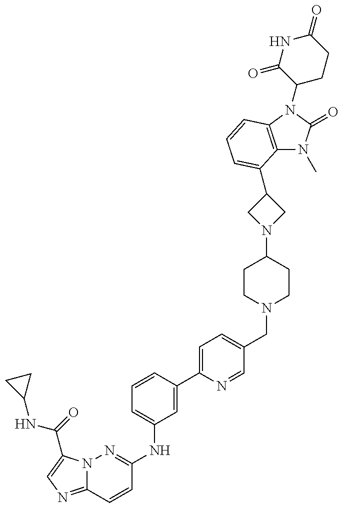 [SMILES: Cn1c(=O)n(C2CCC(=O)NC2=O)c2cccc(C3CN(C4CCN(Cc5ccc(-c6cccc(Nc7ccc8ncc(C(=O)NC9CC9)n8n7)c6)nc5)CC4)C3)c21] |
| K404 | 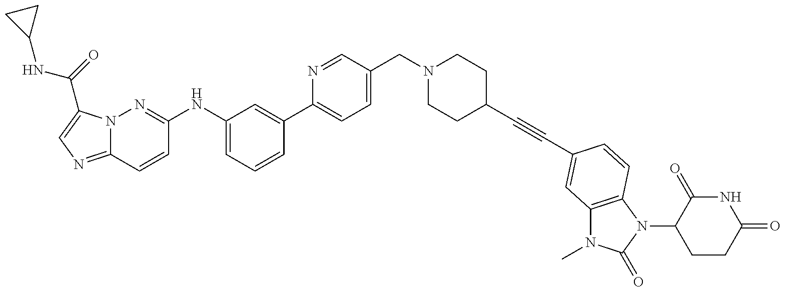 [SMILES: Cn1c(=O)n(C2CCC(=O)NC2=O)c2ccc(C#CC3CCN(Cc4ccc(-c5cccc(Nc6ccc7ncc(C(=O)NC8CC8)n7n6)c5)nc4)CC3)cc21] |
| K405 | 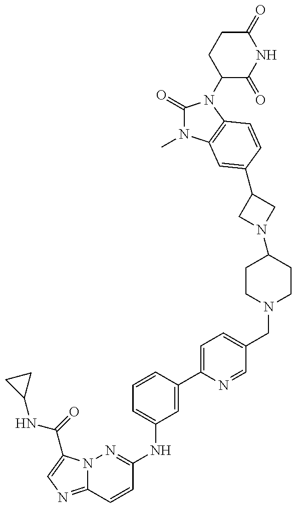 [SMILES: Cn1c(=O)n(C2CCC(=O)NC2=O)c2ccc(C3CN(C4CCN(Cc5ccc(-c6cccc(Nc7ccc8ncc(C(=O)NC9CC9)n8n7)c6)nc5)CC4)C3)cc21] |
| K406 | 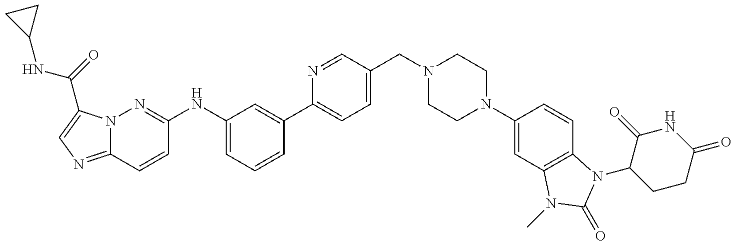 [SMILES: Cn1c(=O)n(C2CCC(=O)NC2=O)c2ccc(N3CCN(Cc4ccc(-c5cccc(Nc6ccc7ncc(C(=O)NC8CC8)n7n6)c5)nc4)CC3)cc21] |
| K407 | 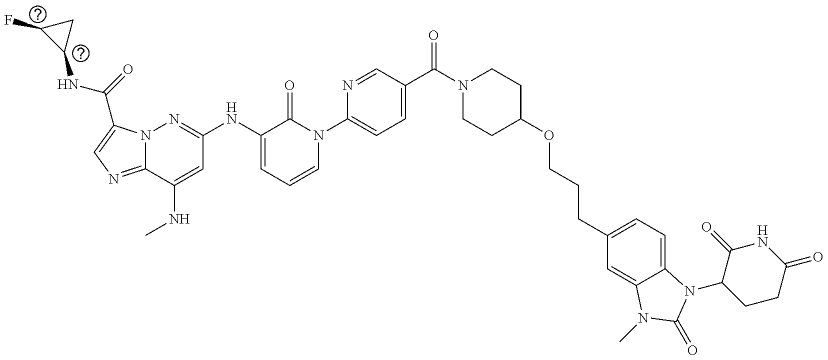 [SMILES: CNc1cc(Nc2cccn(-c3ccc(C(=O)N4CCC(OCCCc5ccc6c(c5)n(C)c(=O)n6C5CCC(=O)NC5=O)CC4)cn3)c2=O)nn2c(C(=O)N[C@@H]3C[C@@H]3F)cnc12] |
| K408 | 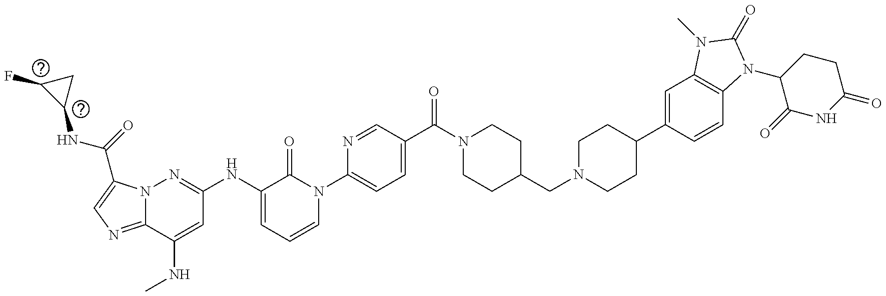 [SMILES: CNc1cc(Nc2cccn(-c3ccc(C(=O)N4CCC(CN5CCC(c6ccc7c(c6)n(C)c(=O)n7C6CCC(=O)NC6=O)CC5)CC4)cn3)c2=O)nn2c(C(=O)N[C@@H]3C[C@@H]3F)cnc12] |
| K409 | 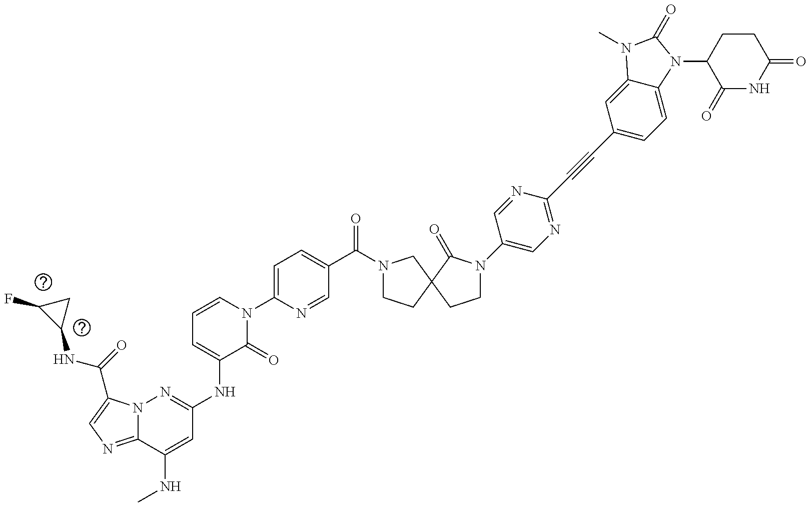 [SMILES: CNc1cc(Nc2cccn(-c3ccc(C(=O)N4CCC5(CCN(c6cnc(C#Cc7ccc8c(c7)n(C)c(=O)n8C7CCC(=O)NC7=O)nc6)C5=O)C4)cn3)c2=O)nn2c(C(=O)N[C@@H]3C[C@@H]3F)cnc12] |
| K410 | 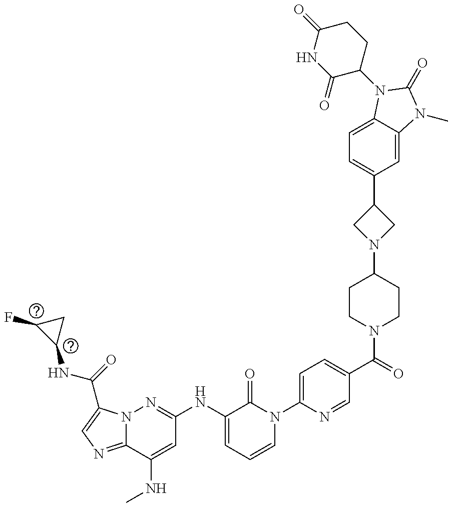 [SMILES: CNc1cc(Nc2cccn(-c3ccc(C(=O)N4CCC(N5CC(c6ccc7c(c6)n(C)c(=O)n7C6CCC(=O)NC6=O)C5)CC4)cn3)c2=O)nn2c(C(=O)N[C@@H]3C[C@@H]3F)cnc12] |
| K411 | 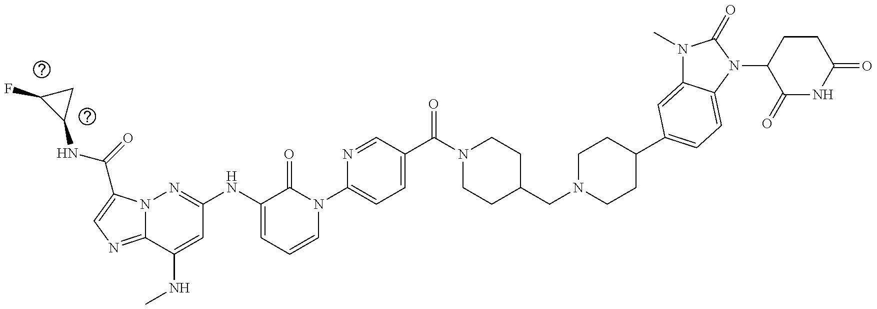 [SMILES: CNc1cc(Nc2cccn(-c3ccc(C(=O)N4CCC(CN5CCC(c6ccc7c(c6)n(C)c(=O)n7C6CCC(=O)NC6=O)CC5)CC4)cn3)c2=O)nn2c(C(=O)N[C@@H]3C[C@@H]3F)cnc12] |
| K412 | 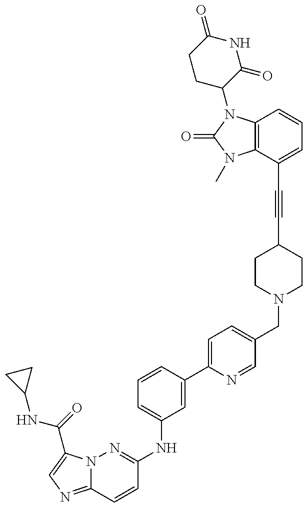 [SMILES: Cn1c(=O)n(C2CCC(=O)NC2=O)c2cccc(C#CC3CCN(Cc4ccc(-c5cccc(Nc6ccc7ncc(C(=O)NC8CC8)n7n6)c5)nc4)CC3)c21] |
| K413 | 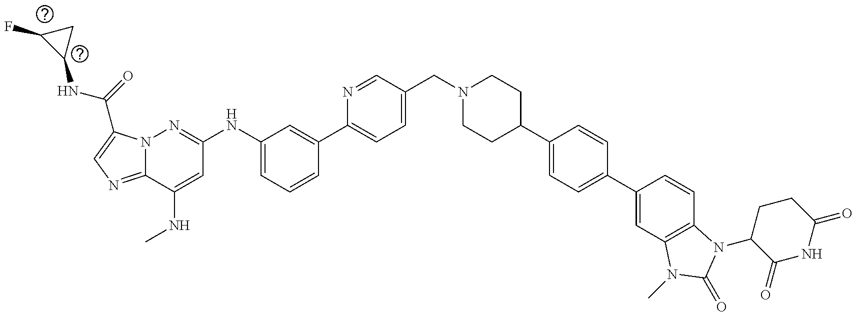 [SMILES: CNc1cc(Nc2cccc(-c3ccc(CN4CCC(c5ccc(-c6ccc7c(c6)n(C)c(=O)n7C6CCC(=O)NC6=O)cc5)CC4)cn3)c2)nn2c(C(=O)N[C@@H]3C[C@@H]3F)cnc12] |
| K414 | 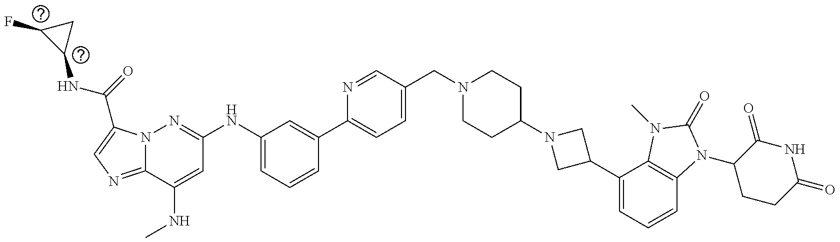 [SMILES: CNc1cc(Nc2cccc(-c3ccc(CN4CCC(N5CC(c6cccc7c6n(C)c(=O)n7C6CCC(=O)NC6=O)C5)CC4)cn3)c2)nn2c(C(=O)N[C@@H]3C[C@@H]3F)cnc12] |
| K415 | 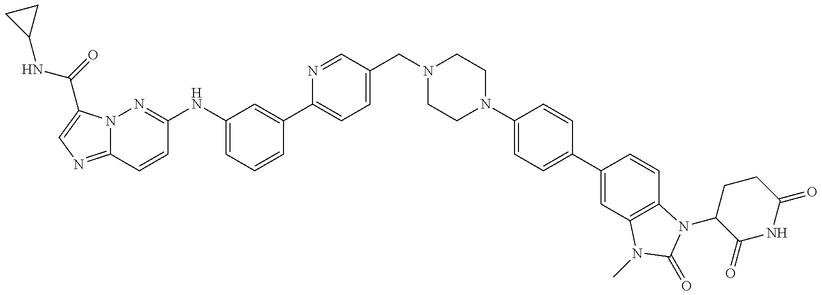 [SMILES: Cn1c(=O)n(C2CCC(=O)NC2=O)c2ccc(-c3ccc(N4CCN(Cc5ccc(-c6cccc(Nc7ccc8ncc(C(=O)NC9CC9)n8n7)c6)nc5)CC4)cc3)cc21] |
| K416 | 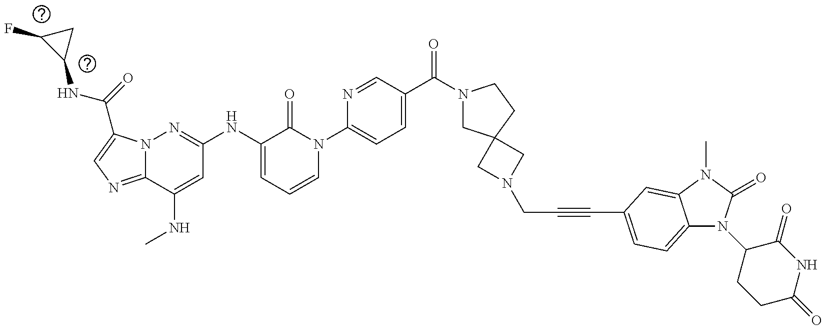 [SMILES: CNc1cc(Nc2cccn(-c3ccc(C(=O)N4CCC5(CN(CC#Cc6ccc7c(c6)n(C)c(=O)n7C6CCC(=O)NC6=O)C5)C4)cn3)c2=O)nn2c(C(=O)N[C@@H]3C[C@@H]3F)cnc12] |
| K417 | 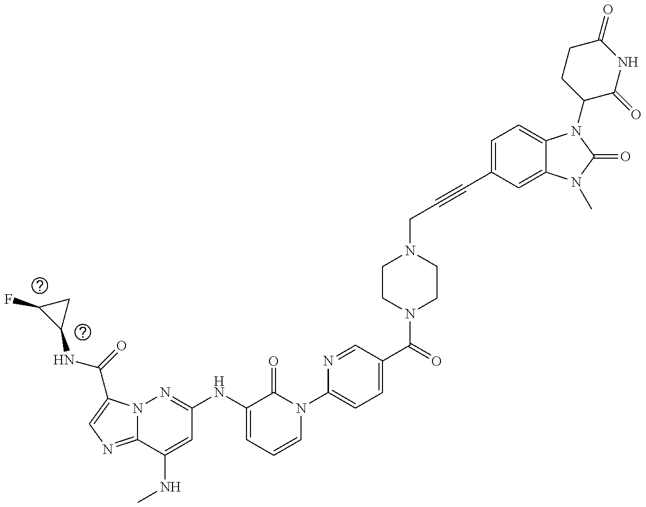 [SMILES: CNc1cc(Nc2cccn(-c3ccc(C(=O)N4CCN(CC#Cc5ccc6c(c5)n(C)c(=O)n6C5CCC(=O)NC5=O)CC4)cn3)c2=O)nn2c(C(=O)N[C@@H]3C[C@@H]3F)cnc12] |
| K418 | 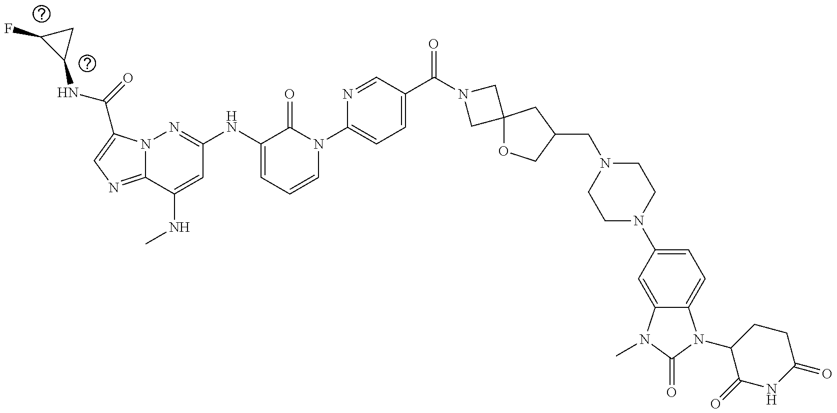 [SMILES: CNc1cc(Nc2cccn(-c3ccc(C(=O)N4CC5(CC(CN6CCN(c7ccc8c(c7)n(C)c(=O)n8C7CCC(=O)NC7=O)CC6)CO5)C4)cn3)c2=O)nn2c(C(=O)N[C@@H]3C[C@@H]3F)cnc12] |
| K419 | 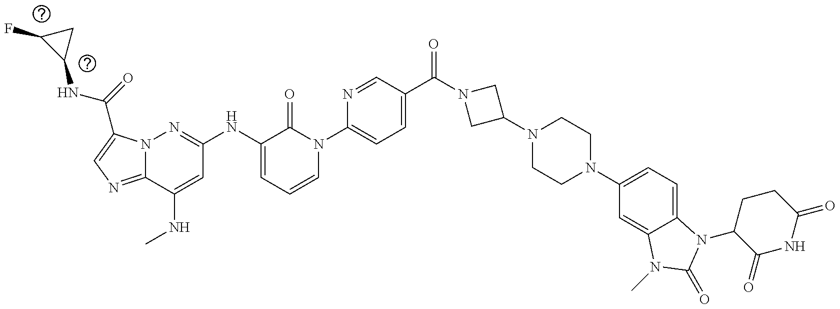 [SMILES: CNc1cc(Nc2cccn(-c3ccc(C(=O)N4CC(N5CCN(c6ccc7c(c6)n(C)c(=O)n7C6CCC(=O)NC6=O)CC5)C4)cn3)c2=O)nn2c(C(=O)N[C@@H]3C[C@@H]3F)cnc12] |
| K420 | 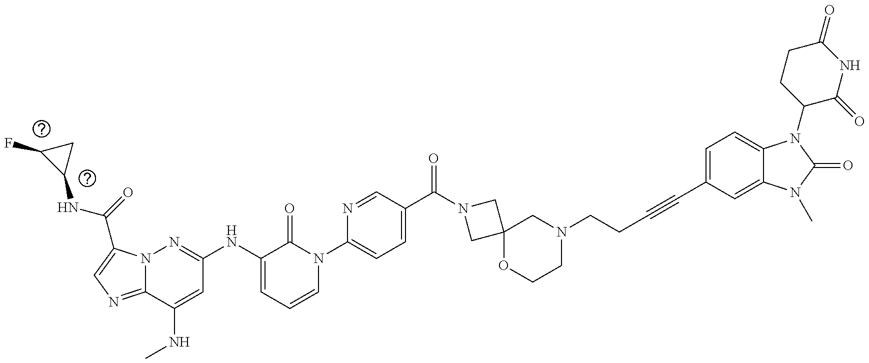 [SMILES: CNc1cc(Nc2cccn(-c3ccc(C(=O)N4CC5(CN(CCC#Cc6ccc7c(c6)n(C)c(=O)n7C6CCC(=O)NC6=O)CCO5)C4)cn3)c2=O)nn2c(C(=O)N[C@@H]3C[C@@H]3F)cnc12] |
| K421 | 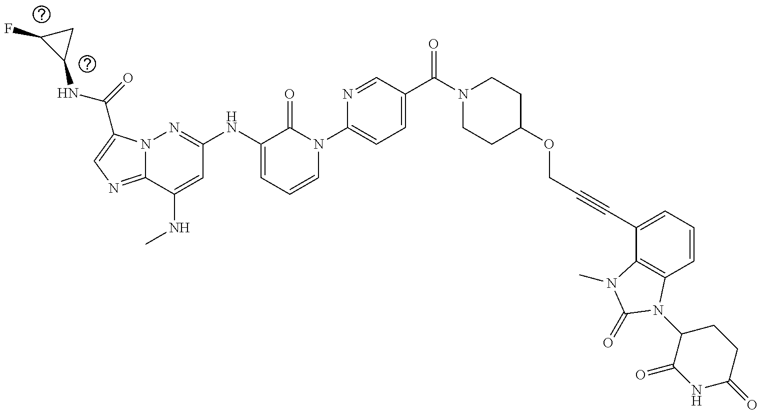 [SMILES: CNc1cc(Nc2cccn(-c3ccc(C(=O)N4CCC(OCC#Cc5cccc6c5n(C)c(=O)n6C5CCC(=O)NC5=O)CC4)cn3)c2=O)nn2c(C(=O)N[C@@H]3C[C@@H]3F)cnc12] |
| K422 | 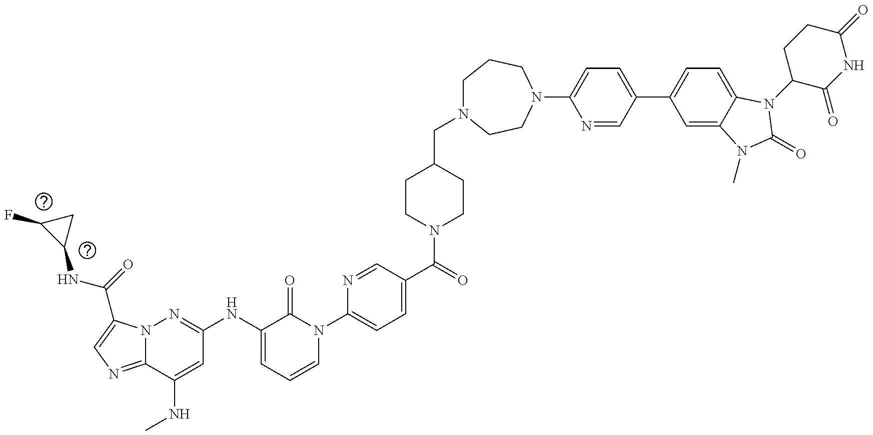 [SMILES: CNc1cc(Nc2cccn(-c3ccc(C(=O)N4CCC(CN5CCCN(c6ccc(-c7ccc8c(c7)n(C)c(=O)n8C7CCC(=O)NC7=O)cn6)CC5)CC4)cn3)c2=O)nn2c(C(=O)N[C@@H]3C[C@@H]3F)cnc12] |
| K423 | 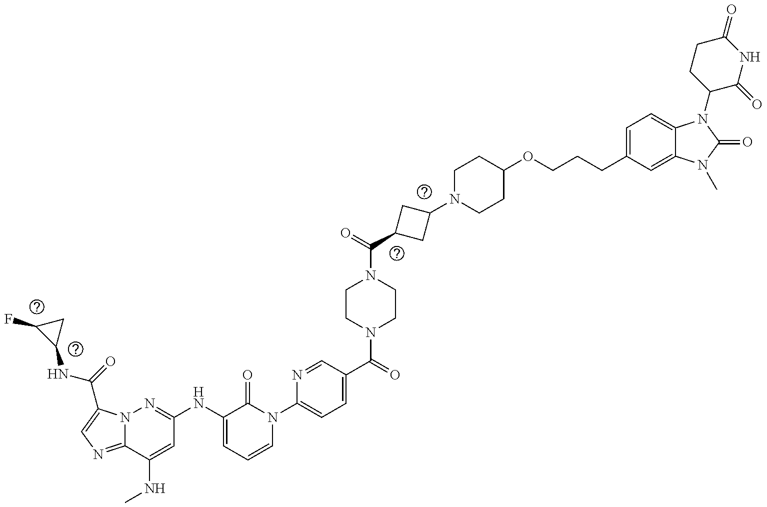 |
| K424 | 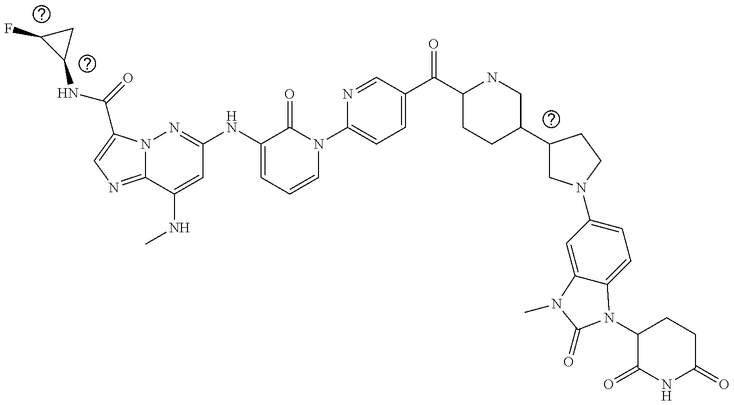 [SMILES: CNc1cc(Nc2cccn(-c3ccc(C(=O)C4CCC(C5CCN(c6ccc7c(c6)n(C)c(=O)n7C6CCC(=O)NC6=O)C5)CN4)cn3)c2=O)nn2c(C(=O)N[C@@H]3C[C@@H]3F)cnc12] |
| K425 | 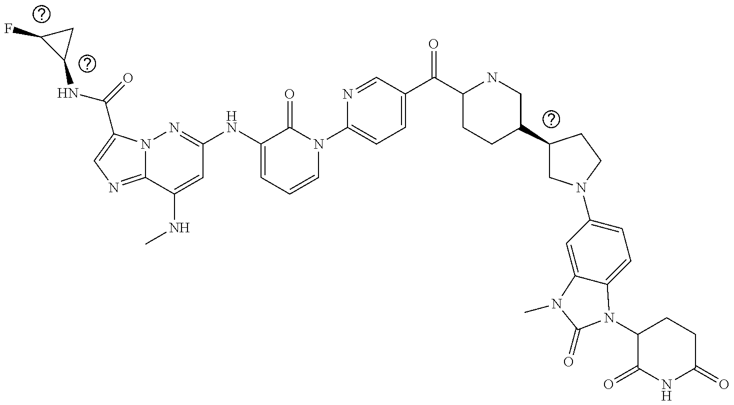 [SMILES: CNc1cc(Nc2cccn(-c3ccc(C(=O)C4CCC([C@H]5CCN(c6ccc7c(c6)n(C)c(=O)n7C6CCC(=O)NC6=O)C5)CN4)cn3)c2=O)nn2c(C(=O)N[C@@H]3C[C@@H]3F)cnc12] |
| K426 | 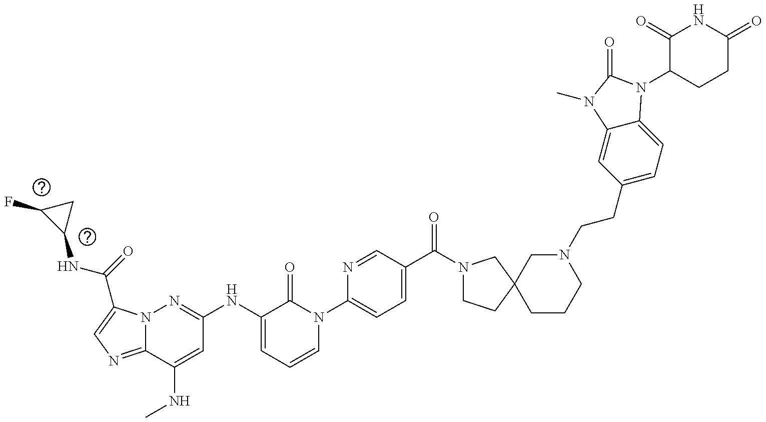 [SMILES: CNc1cc(Nc2cccn(-c3ccc(C(=O)N4CCC5(CCCN(CCc6ccc7c(c6)n(C)c(=O)n7C6CCC(=O)NC6=O)C5)C4)cn3)c2=O)nn2c(C(=O)N[C@@H]3C[C@@H]3F)cnc12] |
| K427 | 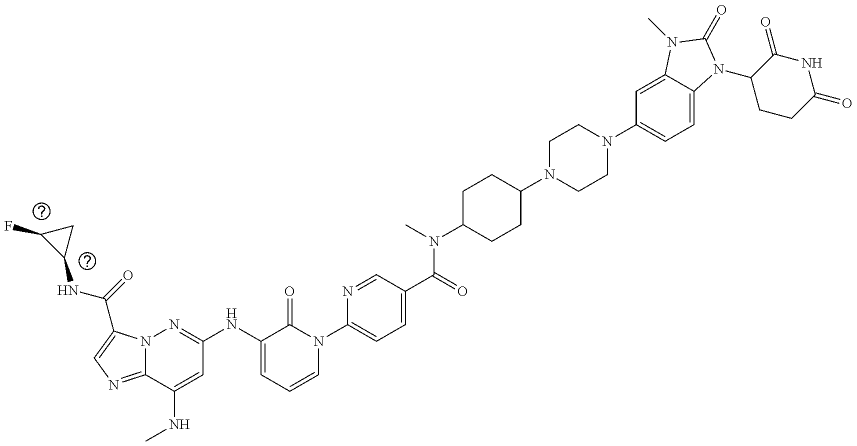 [SMILES: CNc1cc(Nc2cccn(-c3ccc(C(=O)N(C)C4CCC(N5CCN(c6ccc7c(c6)n(C)c(=O)n7C6CCC(=O)NC6=O)CC5)CC4)cn3)c2=O)nn2c(C(=O)N[C@@H]3C[C@@H]3F)cnc12] |
| K428 | 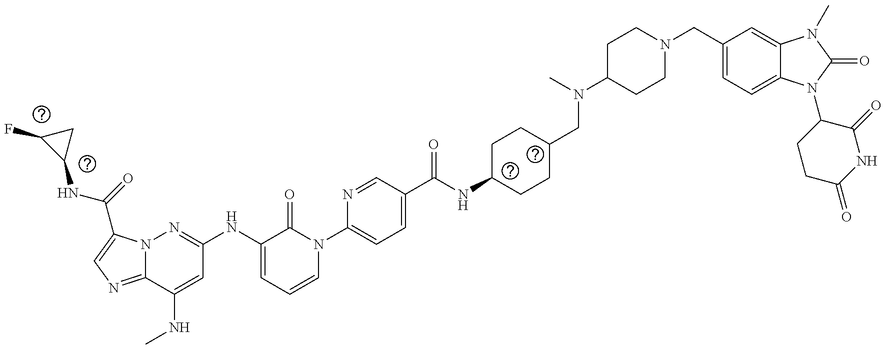 |
| K429 | 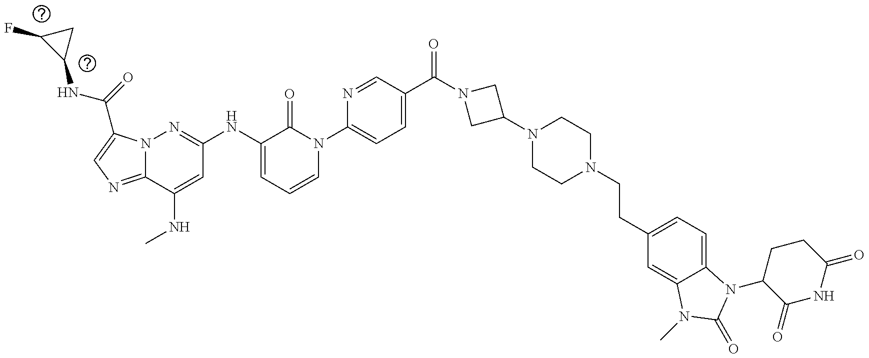 [SMILES: CNc1cc(Nc2cccn(-c3ccc(C(=O)N4CC(N5CCN(CCc6ccc7c(c6)n(C)c(=O)n7C6CCC(=O)NC6=O)CC5)C4)cn3)c2=O)nn2c(C(=O)N[C@@H]3C[C@@H]3F)cnc12] |
| K430 | 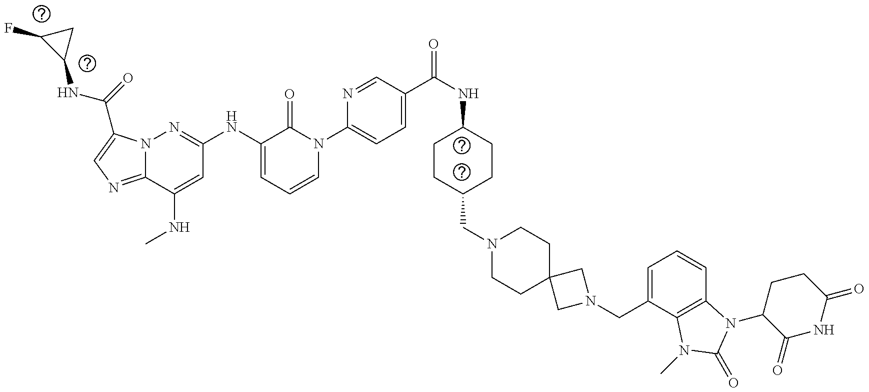 [SMILES: CNc1cc(Nc2cccn(-c3ccc(C(=O)N[C@H]4CC[C@H](CN5CCC6(CC5)CN(Cc5cccc7c5n(C)c(=O)n7C5CCC(=O)NC5=O)C6)CC4)cn3)c2=O)nn2c(C(=O)N[C@@H]3C[C@@H]3F)cnc12] |
| K431 | 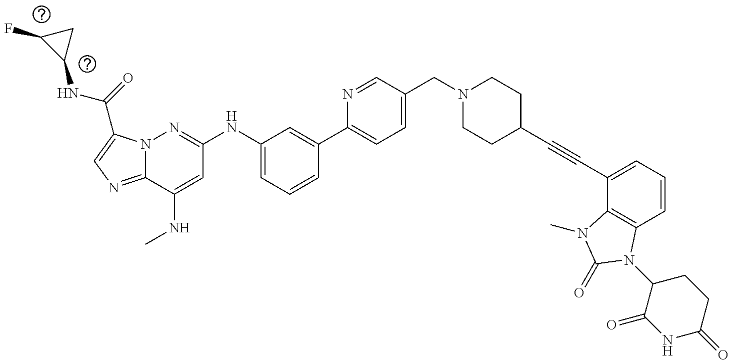 [SMILES: CNc1cc(Nc2cccc(-c3ccc(CN4CCC(C#Cc5cccc6c5n(C)c(=O)n6C5CCC(=O)NC5=O)CC4)cn3)c2)nn2c(C(=O)N[C@@H]3C[C@@H]3F)cnc12] |
| K432 | 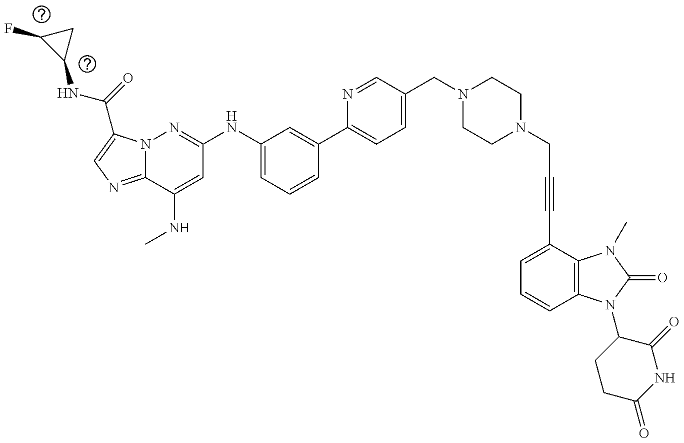 [SMILES: CNc1cc(Nc2cccc(-c3ccc(CN4CCN(CC#Cc5cccc6c5n(C)c(=O)n6C5CCC(=O)NC5=O)CC4)cn3)c2)nn2c(C(=O)N[C@@H]3C[C@@H]3F)cnc12] |
| K433 | 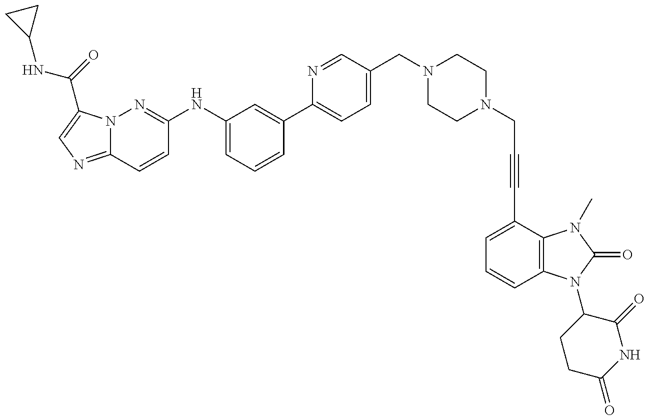 [SMILES: Cn1c(=O)n(C2CCC(=O)NC2=O)c2cccc(C#CCN3CCN(Cc4ccc(-c5cccc(Nc6ccc7ncc(C(=O)NC8CC8)n7n6)c5)nc4)CC3)c21] |
| K434 | 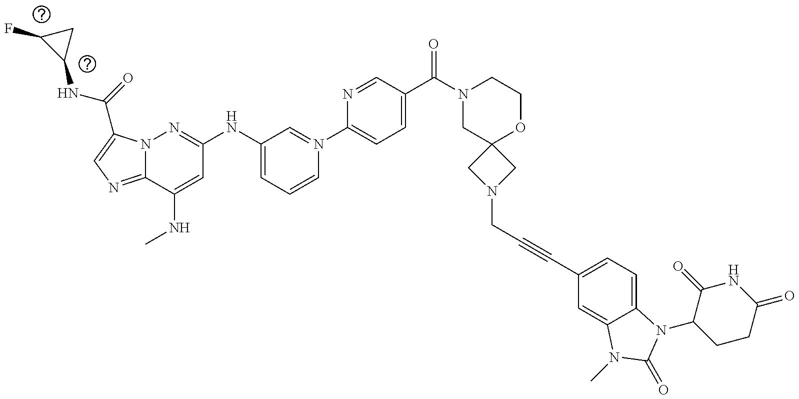 |
| K435 | 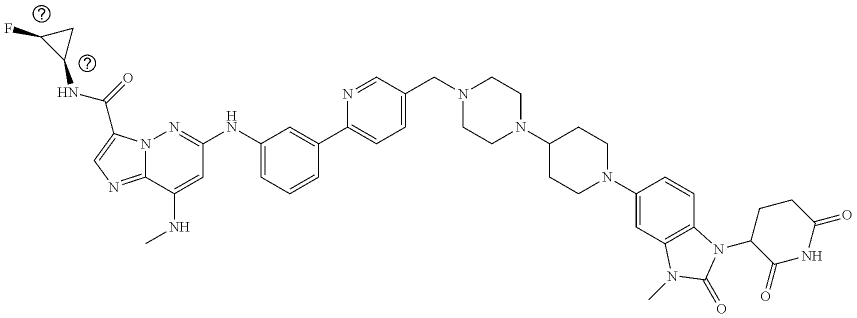 [SMILES: CNc1cc(Nc2cccc(-c3ccc(CN4CCN(C5CCN(c6ccc7c(c6)n(C)c(=O)n7C6CCC(=O)NC6=O)CC5)CC4)cn3)c2)nn2c(C(=O)N[C@@H]3C[C@@H]3F)cnc12] |
| K436 | 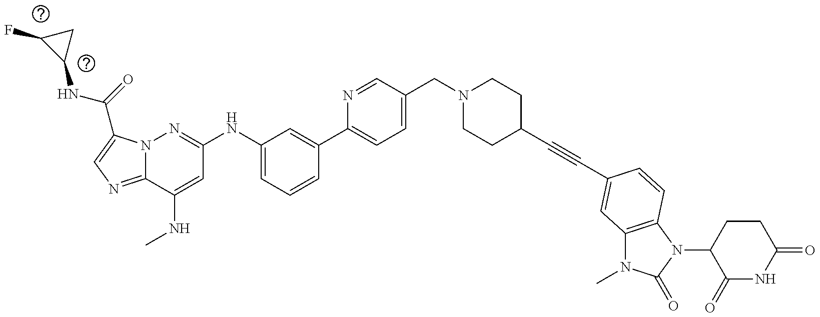 [SMILES: CNc1cc(Nc2cccc(-c3ccc(CN4CCC(C#Cc5ccc6c(c5)n(C)c(=O)n6C5CCC(=O)NC5=O)CC4)cn3)c2)nn2c(C(=O)N[C@@H]3C[C@@H]3F)cnc12] |
| K437 | 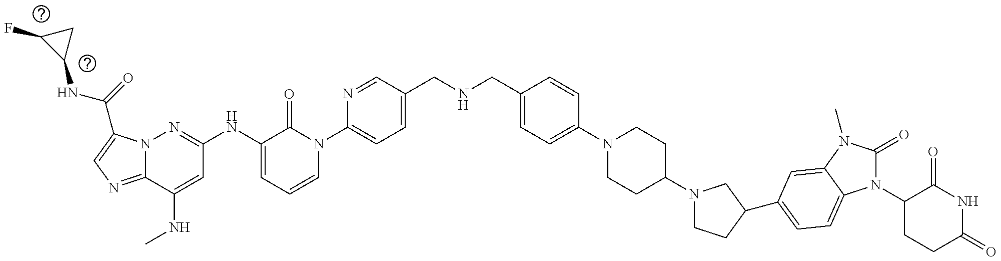 [SMILES: CNc1cc(Nc2cccn(-c3ccc(CNCc4ccc(N5CCC(N6CCC(c7ccc8c(c7)n(C)c(=O)n8C7CCC(=O)NC7=O)C6)CC5)cc4)cn3)c2=O)nn2c(C(=O)N[C@@H]3C[C@@H]3F)cnc12] |
| K438 | 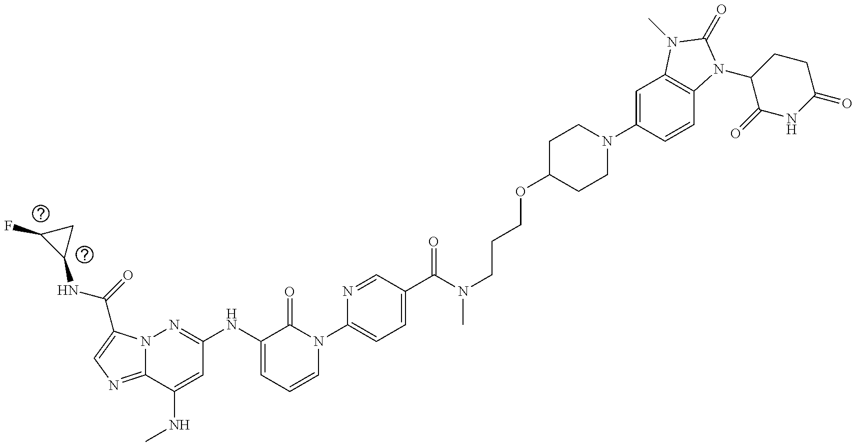 [SMILES: CNc1cc(Nc2cccn(-c3ccc(C(=O)N(C)CCCOC4CCN(c5ccc6c(c5)n(C)c(=O)n6C5CCC(=O)NC5=O)CC4)cn3)c2=O)nn2c(C(=O)N[C@@H]3C[C@@H]3F)cnc12] |
| K439 | 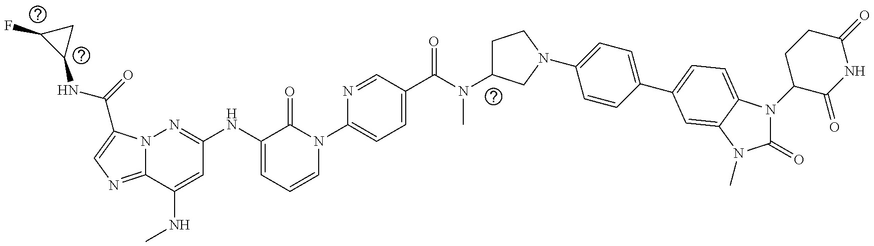 [SMILES: CNc1cc(Nc2cccn(-c3ccc(C(=O)N(C)C4CCN(c5ccc(-c6ccc7c(c6)n(C)c(=O)n7C6CCC(=O)NC6=O)cc5)C4)cn3)c2=O)nn2c(C(=O)N[C@@H]3C[C@@H]3F)cnc12] |
| K440 | 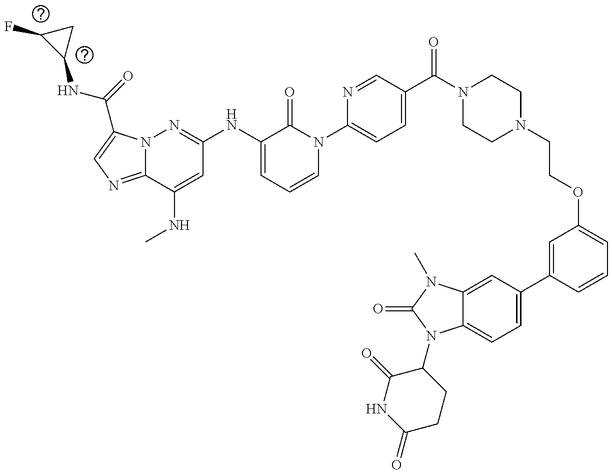 [SMILES: CNc1cc(Nc2cccn(-c3ccc(C(=O)N4CCN(CCOc5cccc(-c6ccc7c(c6)n(C)c(=O)n7C6CCC(=O)NC6=O)c5)CC4)cn3)c2=O)nn2c(C(=O)N[C@@H]3C[C@@H]3F)cnc12] |
| K441 | 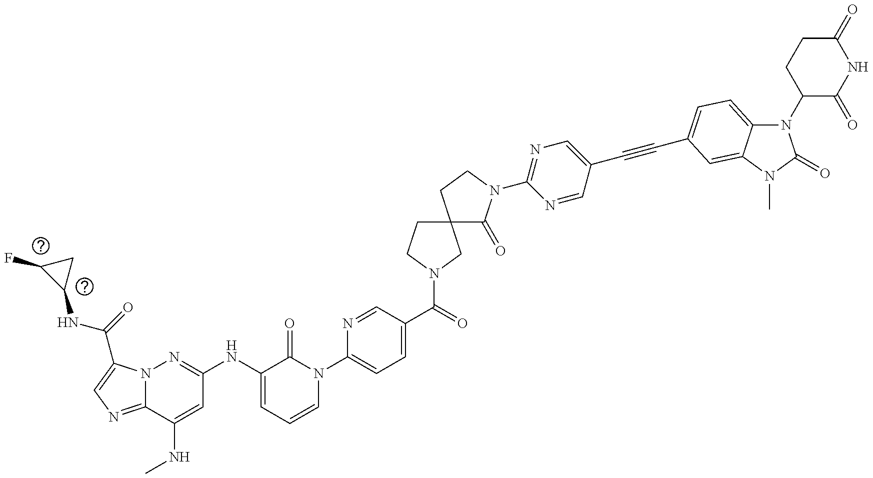 [SMILES: CNc1cc(Nc2cccn(-c3ccc(C(=O)N4CCC5(CCN(c6ncc(C#Cc7ccc8c(c7)n(C)c(=O)n8C7CCC(=O)NC7=O)cn6)C5=O)C4)cn3)c2=O)nn2c(C(=O)N[C@@H]3C[C@@H]3F)cnc12] |
| K442 | 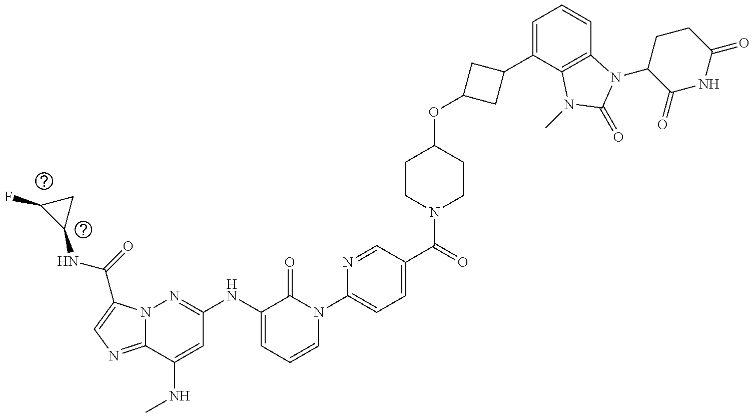 [SMILES: CNc1cc(Nc2cccn(-c3ccc(C(=O)N4CCC(OC5CC(c6cccc7c6n(C)c(=O)n7C6CCC(=O)NC6=O)C5)CC4)cn3)c2=O)nn2c(C(=O)N[C@@H]3C[C@@H]3F)cnc12] |
| K443 | 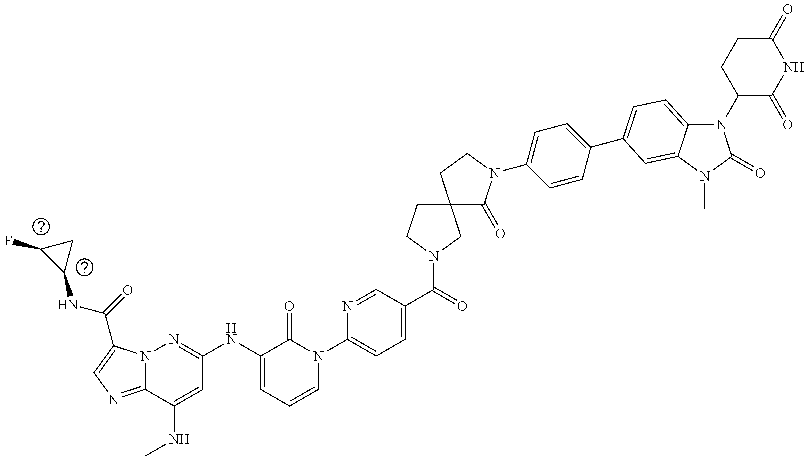 [SMILES: CNc1cc(Nc2cccn(-c3ccc(C(=O)N4CCC5(CCN(c6ccc(-c7ccc8c(c7)n(C)c(=O)n8C7CCC(=O)NC7=O)cc6)C5=O)C4)cn3)c2=O)nn2c(C(=O)N[C@@H]3C[C@@H]3F)cnc12] |
| K444 | 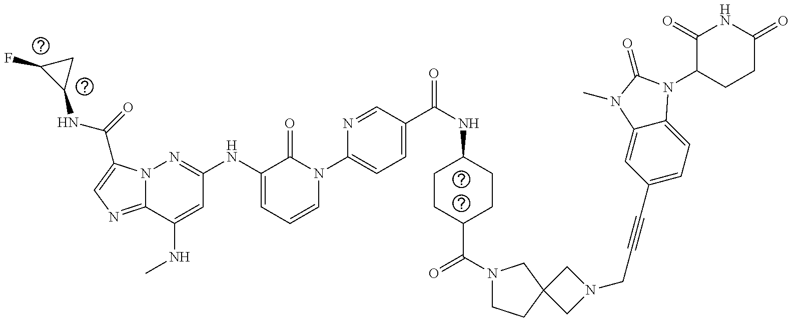 |
| K445 | 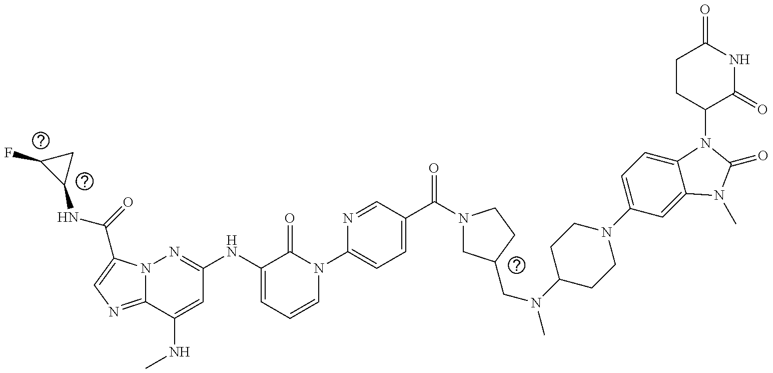 [SMILES: CNc1cc(Nc2cccn(-c3ccc(C(=O)N4CCC(CN(C)C5CCN(c6ccc7c(c6)n(C)c(=O)n7C6CCC(=O)NC6=O)CC5)C4)cn3)c2=O)nn2c(C(=O)N[C@@H]3C[C@@H]3F)cnc12] |
| K446 | 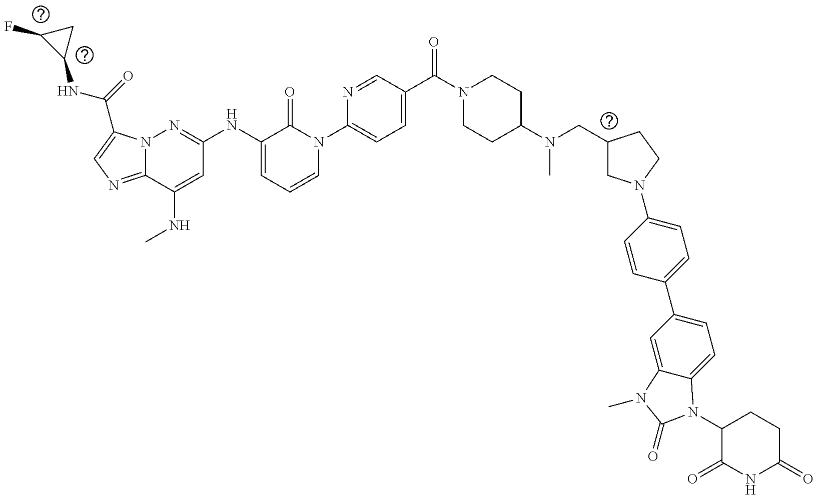 [SMILES: CNc1cc(Nc2cccn(-c3ccc(C(=O)N4CCC(N(C)CC5CCN(c6ccc(-c7ccc8c(c7)n(C)c(=O)n8C7CCC(=O)NC7=O)cc6)C5)CC4)cn3)c2=O)nn2c(C(=O)N[C@@H]3C[C@@H]3F)cnc12] |
| K447 | 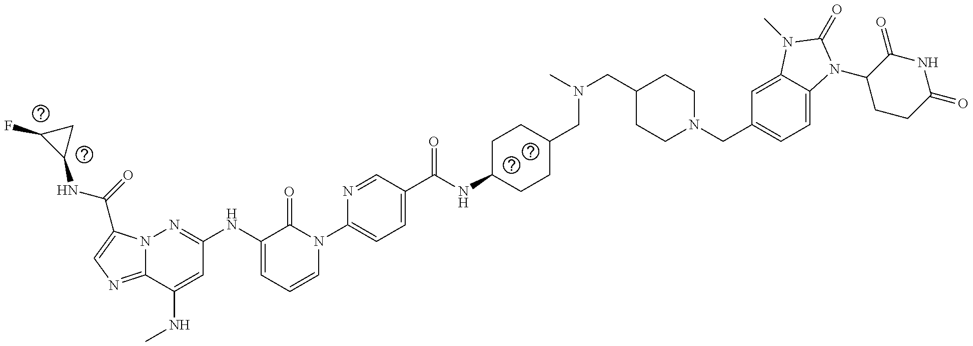 |
| K448 | 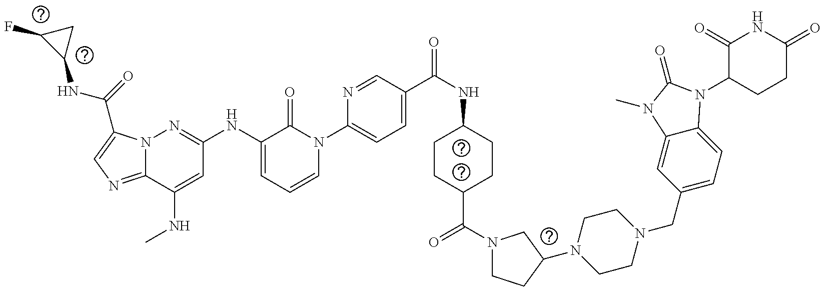 |
| K449 | 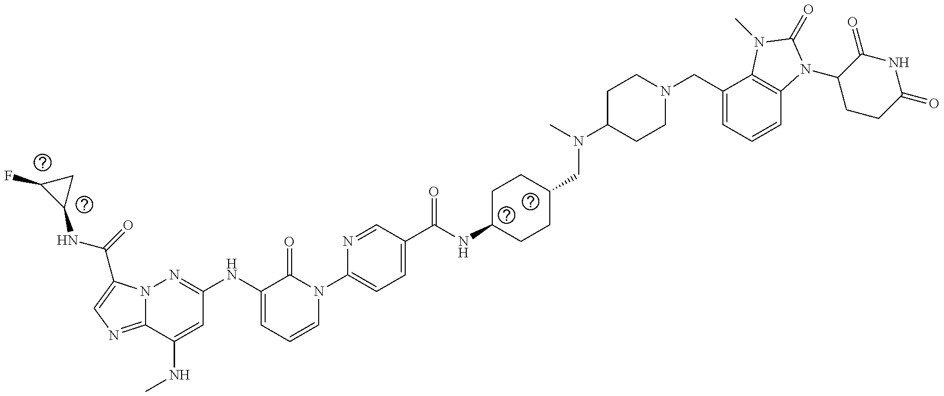 [SMILES: CNc1cc(Nc2cccn(-c3ccc(C(=O)N[C@H]4CC[C@H](CN(C)C5CCN(Cc6cccc7c6n(C)c(=O)n7C6CCC(=O)NC6=O)CC5)CC4)cn3)c2=O)nn2c(C(=O)N[C@@H]3C[C@@H]3F)cnc12] |
| K450 | 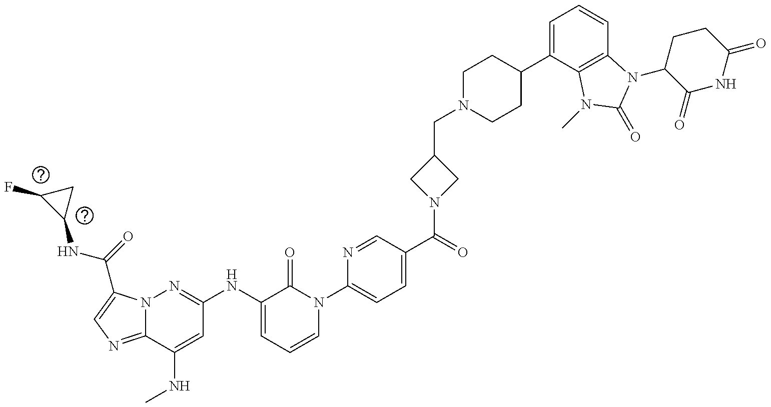 [SMILES: CNc1cc(Nc2cccn(-c3ccc(C(=O)N4CC(CN5CCC(c6cccc7c6n(C)c(=O)n7C6CCC(=O)NC6=O)CC5)C4)cn3)c2=O)nn2c(C(=O)N[C@@H]3C[C@@H]3F)cnc12] |
| K451 | 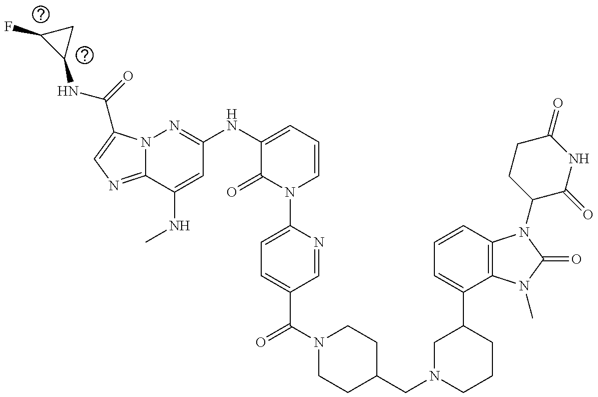 [SMILES: CNc1cc(Nc2cccn(-c3ccc(C(=O)N4CCC(CN5CCCC(c6cccc7c6n(C)c(=O)n7C6CCC(=O)NC6=O)C5)CC4)cn3)c2=O)nn2c(C(=O)N[C@@H]3C[C@@H]3F)cnc12] |
| K452 | 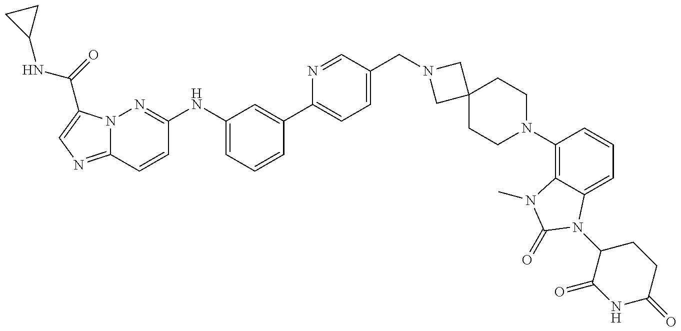 [SMILES: Cn1c(=O)n(C2CCC(=O)NC2=O)c2cccc(N3CCC4(CC3)CN(Cc3ccc(-c5cccc(Nc6ccc7ncc(C(=O)NC8CC8)n7n6)c5)nc3)C4)c21] |
| K453 | 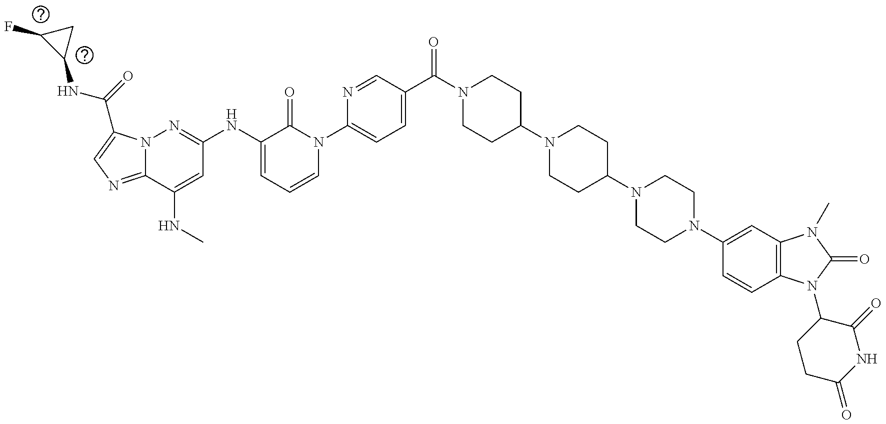 [SMILES: CNc1cc(Nc2cccn(-c3ccc(C(=O)N4CCC(N5CCC(N6CCN(c7ccc8c(c7)n(C)c(=O)n8C7CCC(=O)NC7=O)CC6)CC5)CC4)cn3)c2=O)nn2c(C(=O)N[C@@H]3C[C@@H]3F)cnc12] |
| K454 | 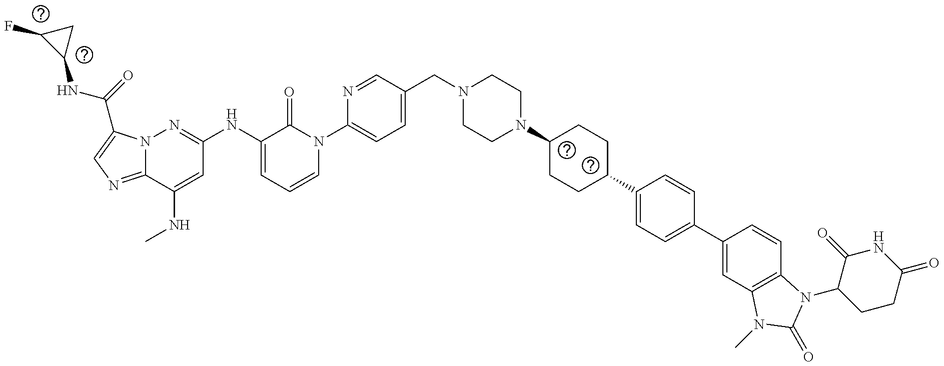 [SMILES: CNc1cc(Nc2cccn(-c3ccc(CN4CCN([C@H]5CC[C@H](c6ccc(-c7ccc8c(c7)n(C)c(=O)n8C7CCC(=O)NC7=O)cc6)CC5)CC4)cn3)c2=O)nn2c(C(=O)N[C@@H]3C[C@@H]3F)cnc12] |
| K455 | 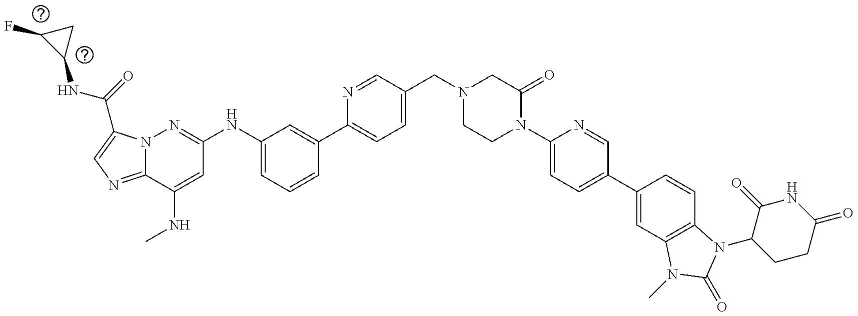 [SMILES: CNc1cc(Nc2cccc(-c3ccc(CN4CCN(c5ccc(-c6ccc7c(c6)n(C)c(=O)n7C6CCC(=O)NC6=O)cn5)C(=O)C4)cn3)c2)nn2c(C(=O)N[C@@H]3C[C@@H]3F)cnc12] |
| K456 | 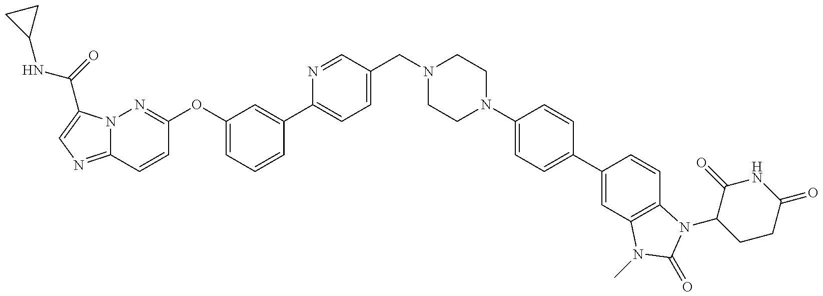 [SMILES: Cn1c(=O)n(C2CCC(=O)NC2=O)c2ccc(-c3ccc(N4CCN(Cc5ccc(-c6cccc(Oc7ccc8ncc(C(=O)NC9CC9)n8n7)c6)nc5)CC4)cc3)cc21] |
| K457 | 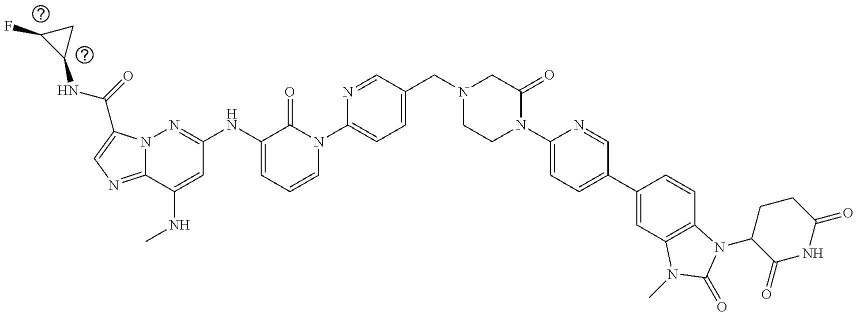 [SMILES: CNc1cc(Nc2cccn(-c3ccc(CN4CCN(c5ccc(-c6ccc7c(c6)n(C)c(=O)n7C6CCC(=O)NC6=O)cn5)C(=O)C4)cn3)c2=O)nn2c(C(=O)N[C@@H]3C[C@@H]3F)cnc12] |
| K458 | 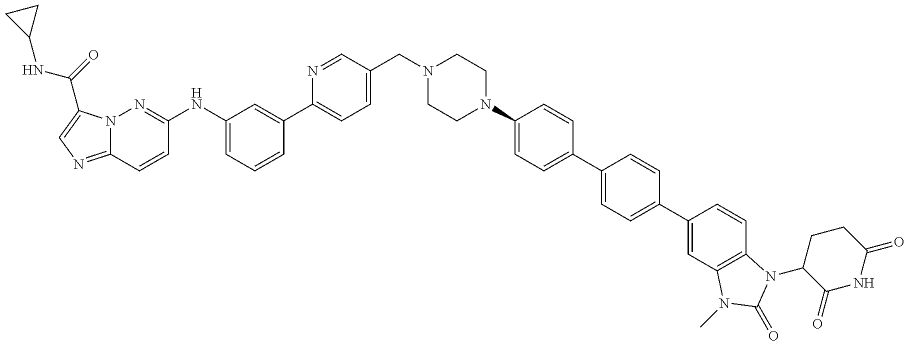 |
| K459 | 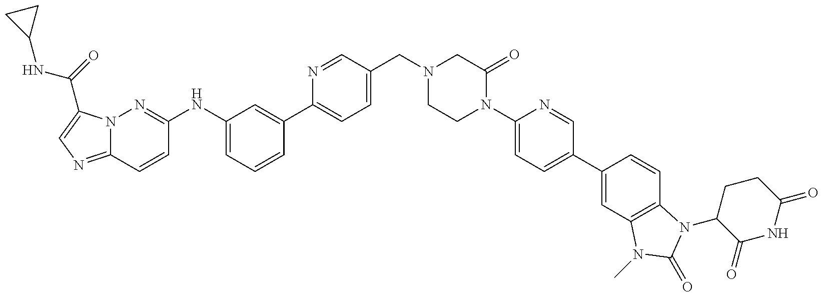 [SMILES: Cn1c(=O)n(C2CCC(=O)NC2=O)c2ccc(-c3ccc(N4CCN(Cc5ccc(-c6cccc(Nc7ccc8ncc(C(=O)NC9CC9)n8n7)c6)nc5)CC4=O)nc3)cc21] |
| K460 | 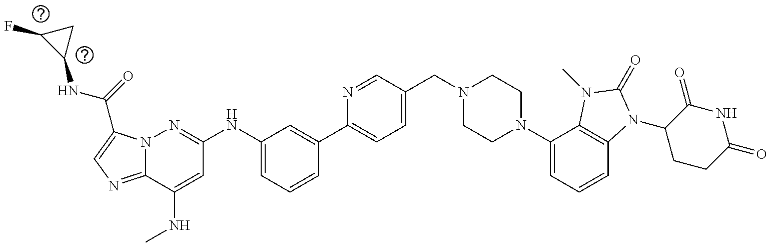 [SMILES: CNc1cc(Nc2cccc(-c3ccc(CN4CCN(c5cccc6c5n(C)c(=O)n6C5CCC(=O)NC5=O)CC4)cn3)c2)nn2c(C(=O)N[C@@H]3C[C@@H]3F)cnc12] |
| K461 | 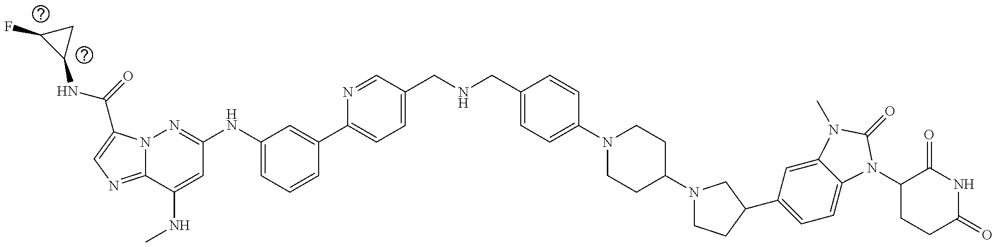 [SMILES: CNc1cc(Nc2cccc(-c3ccc(CNCc4ccc(N5CCC(N6CCC(c7ccc8c(c7)n(C)c(=O)n8C7CCC(=O)NC7=O)C6)CC5)cc4)cn3)c2)nn2c(C(=O)N[C@@H]3C[C@@H]3F)cnc12] |
| K462 | 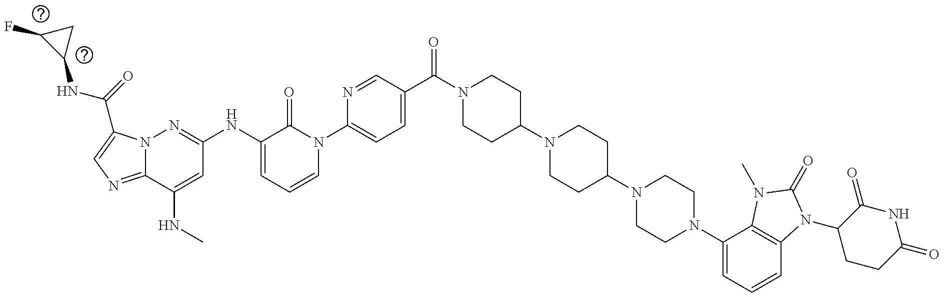 [SMILES: CNc1cc(Nc2cccn(-c3ccc(C(=O)N4CCC(N5CCC(N6CCN(c7cccc8c7n(C)c(=O)n8C7CCC(=O)NC7=O)CC6)CC5)CC4)cn3)c2=O)nn2c(C(=O)N[C@@H]3C[C@@H]3F)cnc12] |
| K463 | 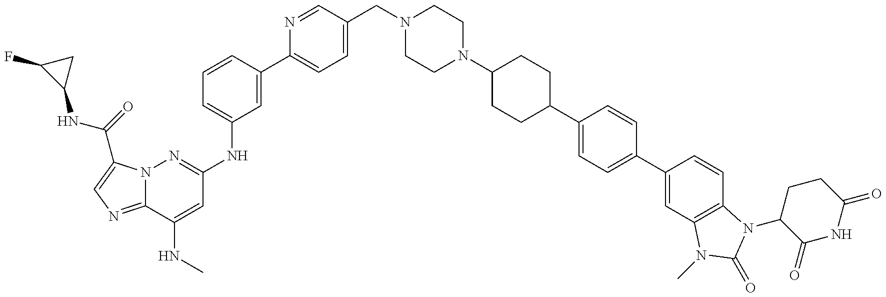 [SMILES: CNc1cc(Nc2cccc(-c3ccc(CN4CCN(C5CCC(c6ccc(-c7ccc8c(c7)n(C)c(=O)n8C7CCC(=O)NC7=O)cc6)CC5)CC4)cn3)c2)nn2c(C(=O)N[C@@H]3C[C@@H]3F)cnc12] |
| K464 | 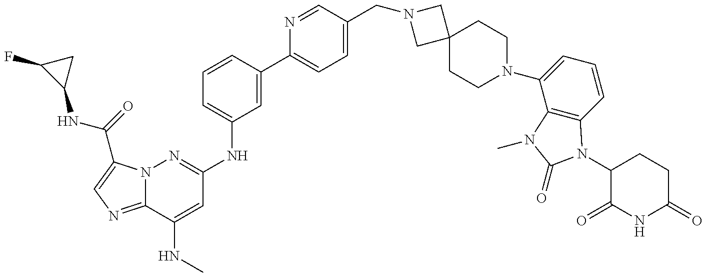 [SMILES: CNc1cc(Nc2cccc(-c3ccc(CN4CC5(CCN(c6cccc7c6n(C)c(=O)n7C6CCC(=O)NC6=O)CC5)C4)cn3)c2)nn2c(C(=O)N[C@@H]3C[C@@H]3F)cnc12] |
| K465 | 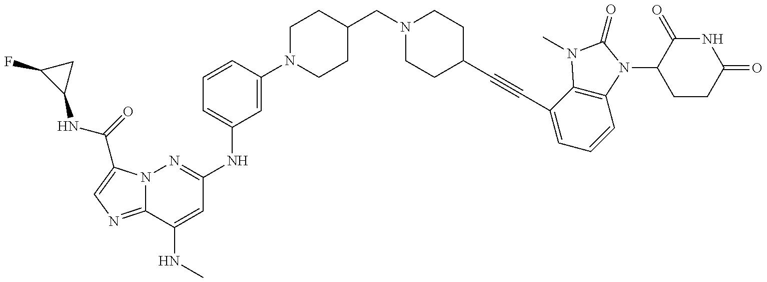 [SMILES: CNc1cc(Nc2cccc(N3CCC(CN4CCC(C#Cc5cccc6c5n(C)c(=O)n6C5CCC(=O)NC5=O)CC4)CC3)c2)nn2c(C(=O)N[C@@H]3C[C@@H]3F)cnc12] |
| K466 | 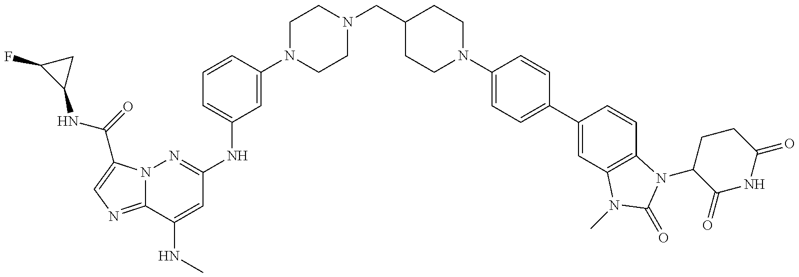 [SMILES: CNc1cc(Nc2cccc(N3CCN(CC4CCN(c5ccc(-c6ccc7c(c6)n(C)c(=O)n7C6CCC(=O)NC6=O)cc5)CC4)CC3)c2)nn2c(C(=O)N[C@@H]3C[C@@H]3F)cnc12] |
| K467 | 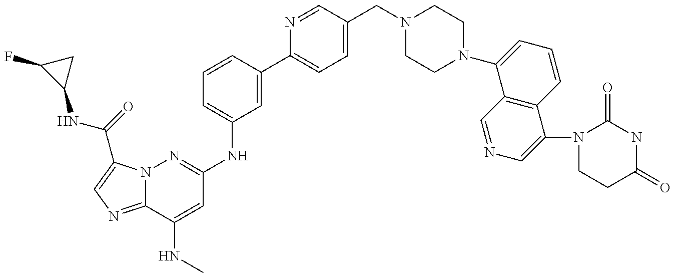 [SMILES: CNc1cc(Nc2cccc(-c3ccc(CN4CCN(c5cccc6c(N7CCC(=O)NC7=O)cncc56)CC4)cn3)c2)nn2c(C(=O)N[C@@H]3C[C@@H]3F)cnc12] |
| K468 | 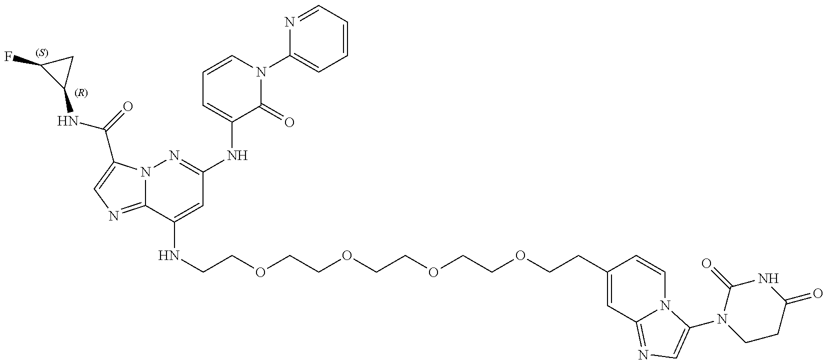 |
| K469 | 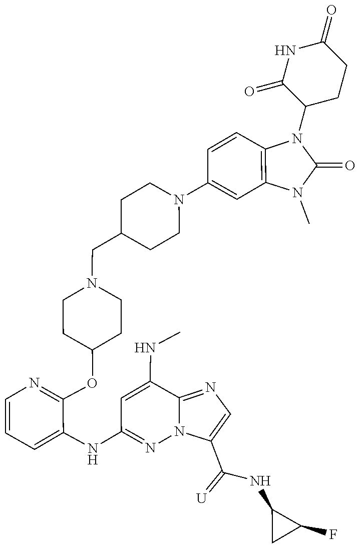 |
| K470 | 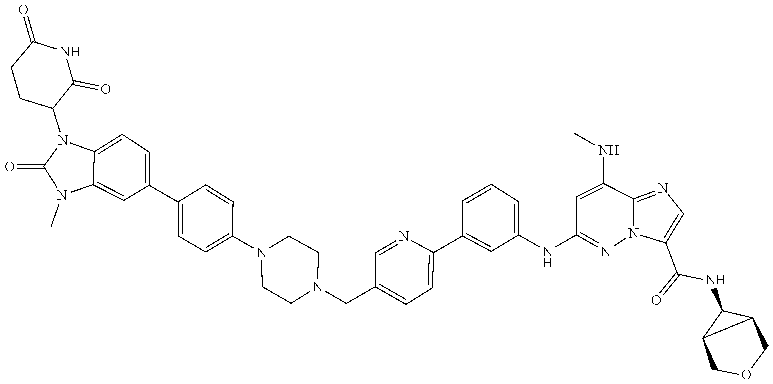 [SMILES: CNc1cc(Nc2cccc(-c3ccc(CN4CCN(c5ccc(-c6ccc7c(c6)n(C)c(=O)n7C6CCC(=O)NC6=O)cc5)CC4)cn3)c2)nn2c(C(=O)N[C@@H]3[C@@H]4COC[C@@H]43)cnc12] |
| K471 | 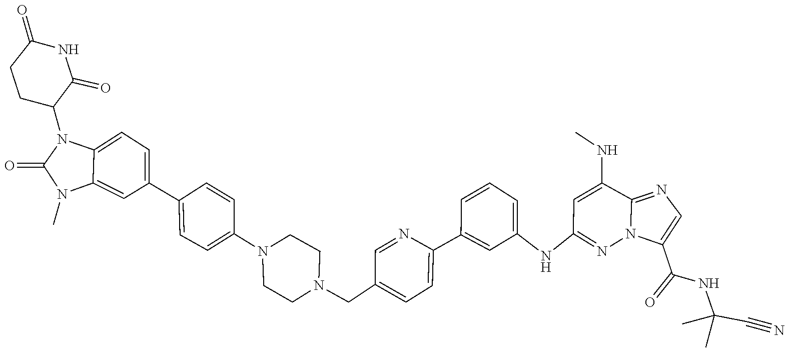 [SMILES: CNc1cc(Nc2cccc(-c3ccc(CN4CCN(c5ccc(-c6ccc7c(c6)n(C)c(=O)n7C6CCC(=O)NC6=O)cc5)CC4)cn3)c2)nn2c(C(=O)NC(C)(C)C#N)cnc12] |
| K472 | 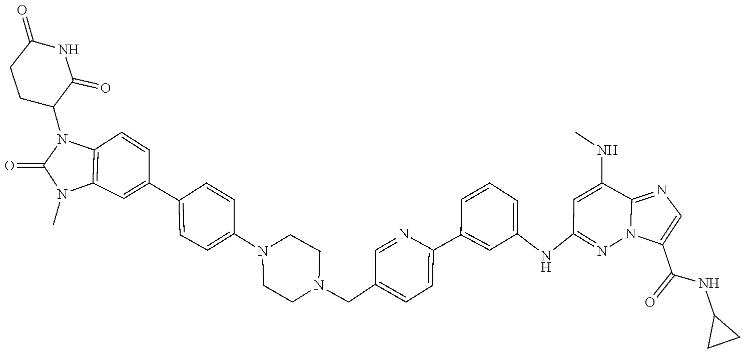 [SMILES: CNc1cc(Nc2cccc(-c3ccc(CN4CCN(c5ccc(-c6ccc7c(c6)n(C)c(=O)n7C6CCC(=O)NC6=O)cc5)CC4)cn3)c2)nn2c(C(=O)NC3CC3)cnc12] |
| K473 | 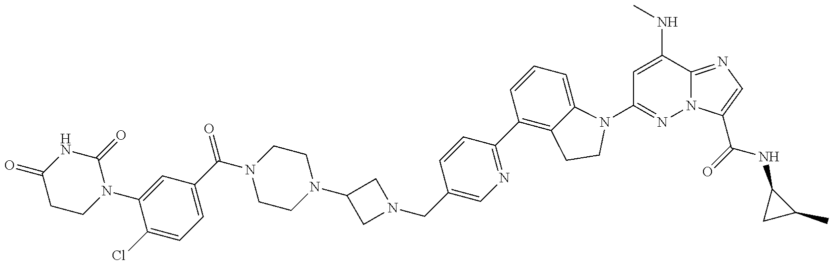 [SMILES: CNc1cc(N2CCc3c(-c4ccc(CN5CC(N6CCN(C(=O)c7ccc(Cl)c(N8CCC(=O)NC8=O)c7)CC6)C5)cn4)cccc32)nn2c(C(=O)N[C@@H]3C[C@@H]3C)cnc12] |
| K474 | 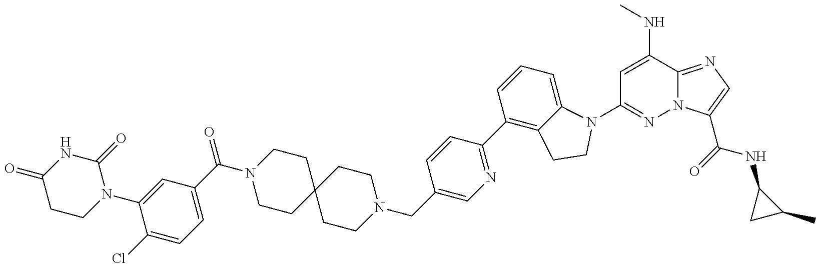 [SMILES: CNc1cc(N2CCc3c(-c4ccc(CN5CCC6(CC5)CCN(C(=O)c5ccc(Cl)c(N7CCC(=O)NC7=O)c5)CC6)cn4)cccc32)nn2c(C(=O)N[C@@H]3C[C@@H]3C)cnc12] |
| K475 | 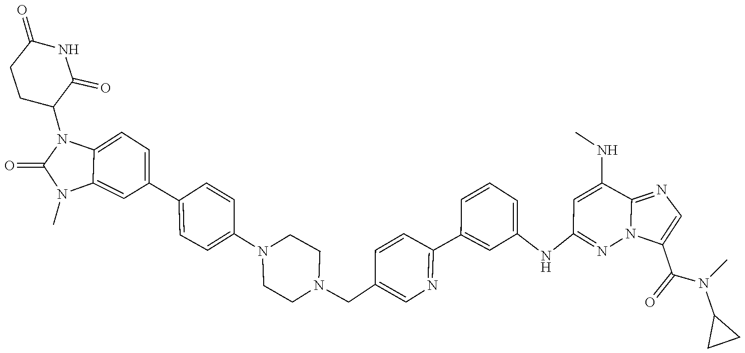 [SMILES: CNc1cc(Nc2cccc(-c3ccc(CN4CCN(c5ccc(-c6ccc7c(c6)n(C)c(=O)n7C6CCC(=O)NC6=O)cc5)CC4)cn3)c2)nn2c(C(=O)N(C)C3CC3)cnc12] |
| K476 | 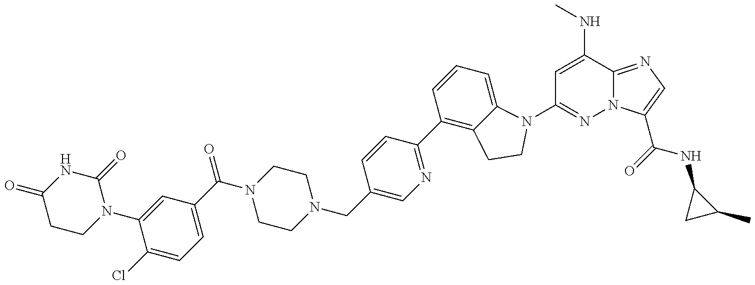 [SMILES: CNc1cc(N2CCc3c(-c4ccc(CN5CCN(C(=O)c6ccc(Cl)c(N7CCC(=O)NC7=O)c6)CC5)cn4)cccc32)nn2c(C(=O)N[C@@H]3C[C@@H]3C)cnc12] |
| K477 | 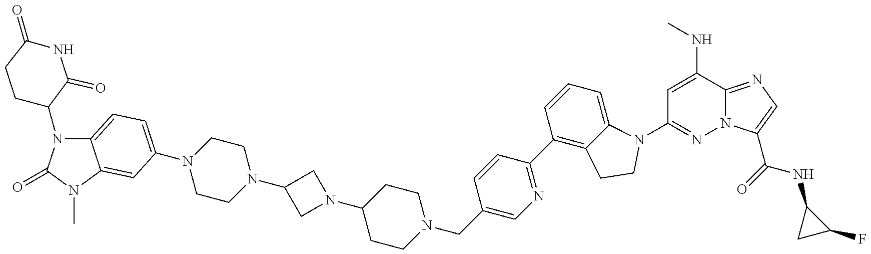 [SMILES: CNc1cc(N2CCc3c(-c4ccc(CN5CCC(N6CC(N7CCN(c8ccc9c(c8)n(C)c(=O)n9C8CCC(=O)NC8=O)CC7)C6)CC5)cn4)cccc32)nn2c(C(=O)N[C@@H]3C[C@@H]3F)cnc12] |
| | 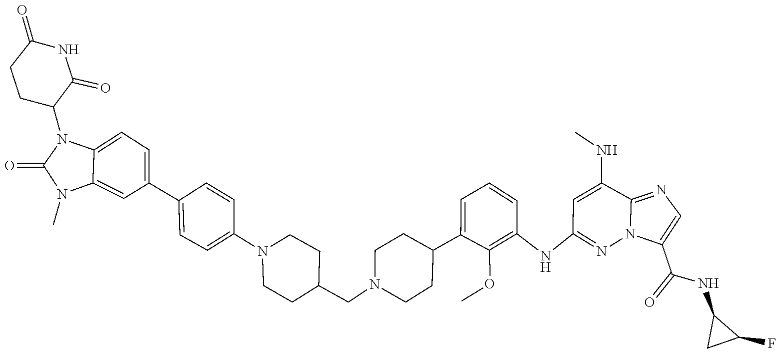 [SMILES: CNc1cc(Nc2cccc(C3CCN(CC4CCN(c5ccc(-c6ccc7c(c6)n(C)c(=O)n7C6CCC(=O)NC6=O)cc5)CC4)CC3)c2OC)nn2c(C(=O)N[C@@H]3C[C@@H]3F)cnc12] |
| K479 | 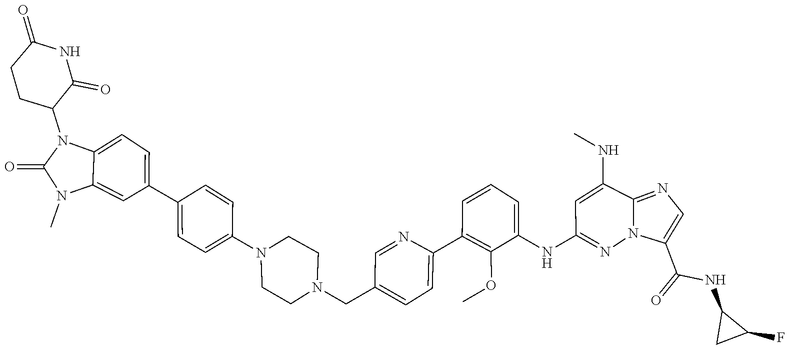 [SMILES: CNc1cc(Nc2cccc(-c3ccc(CN4CCN(c5ccc(-c6ccc7c(c6)n(C)c(=O)n7C6CCC(=O)NC6=O)cc5)CC4)cn3)c2OC)nn2c(C(=O)N[C@@H]3C[C@@H]3F)cnc12] |
| K480 | 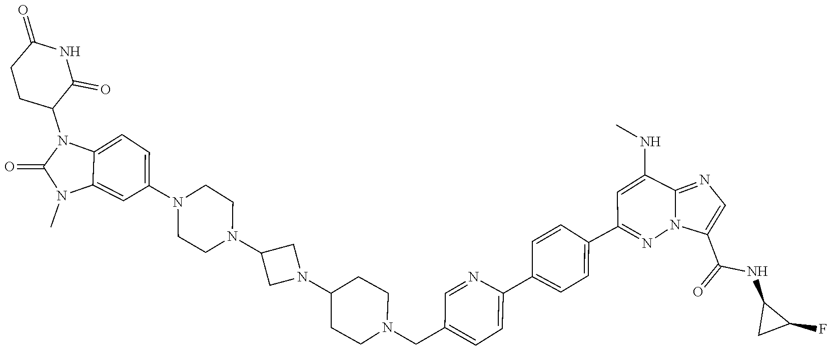 [SMILES: CNc1cc(-c2ccc(-c3ccc(CN4CCC(N5CC(N6CCN(c7ccc8c(c7)n(C)c(=O)n8C7CCC(=O)NC7=O)CC6)C5)CC4)cn3)cc2)nn2c(C(=O)N[C@@H]3C[C@@H]3F)cnc12] |
| K481 | 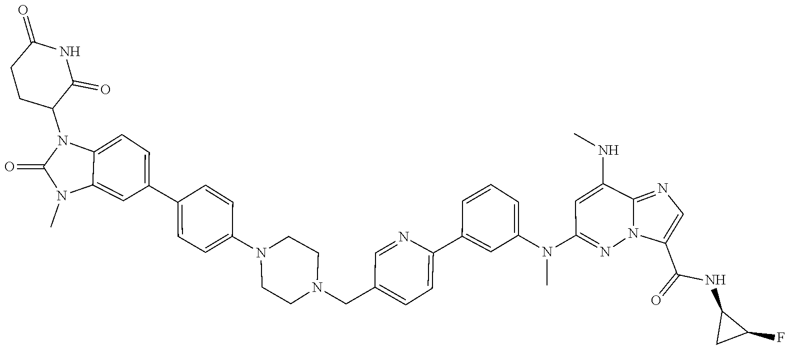 [SMILES: CNc1cc(N(C)c2cccc(-c3ccc(CN4CCN(c5ccc(-c6ccc7c(c6)n(C)c(=O)n7C6CCC(=O)NC6=O)cc5)CC4)cn3)c2)nn2c(C(=O)N[C@@H]3C[C@@H]3F)cnc12] |
| K482 | 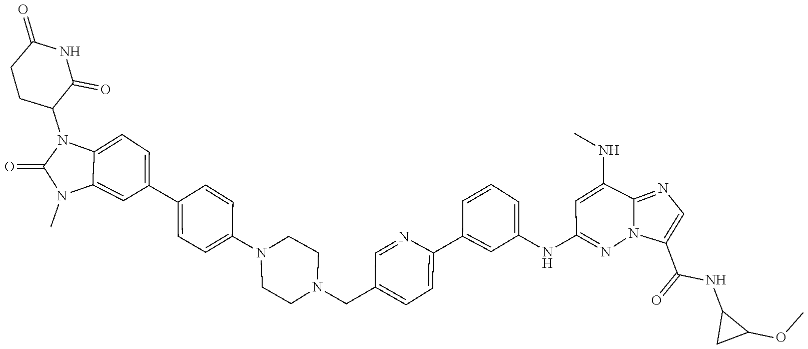 [SMILES: CNc1cc(Nc2cccc(-c3ccc(CN4CCN(c5ccc(-c6ccc7c(c6)n(C)c(=O)n7C6CCC(=O)NC6=O)cc5)CC4)cn3)c2)nn2c(C(=O)NC3CC3OC)cnc12] |
| K483 | 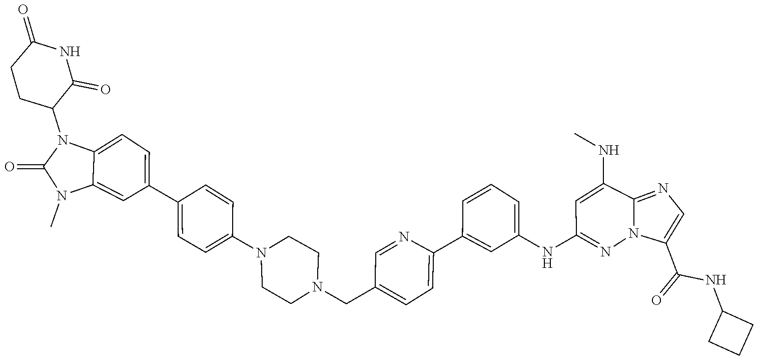 [SMILES: CNc1cc(Nc2cccc(-c3ccc(CN4CCN(c5ccc(-c6ccc7c(c6)n(C)c(=O)n7C6CCC(=O)NC6=O)cc5)CC4)cn3)c2)nn2c(C(=O)NC3CCC3)cnc12] |
| K484 | 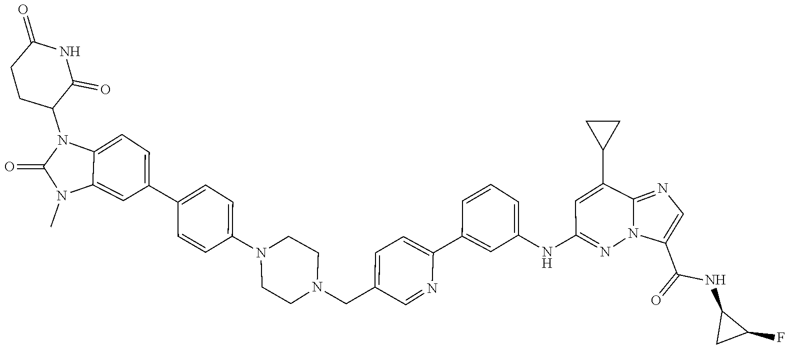 [SMILES: Cn1c(=O)n(C2CCC(=O)NC2=O)c2ccc(-c3ccc(N4CCN(Cc5ccc(-c6cccc(Nc7cc(C8CC8)c8ncc(C(=O)N[C@@H]9C[C@@H]9F)n8n7)c6)nc5)CC4)cc3)cc21] |
| K485 | 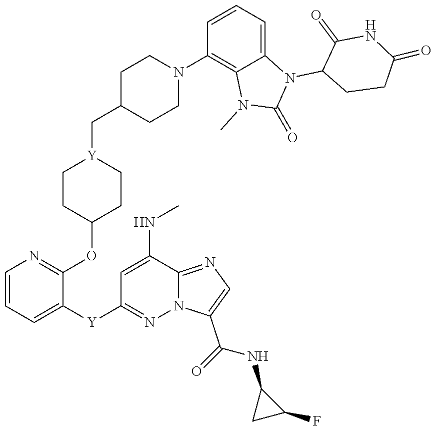 |
| K486 | 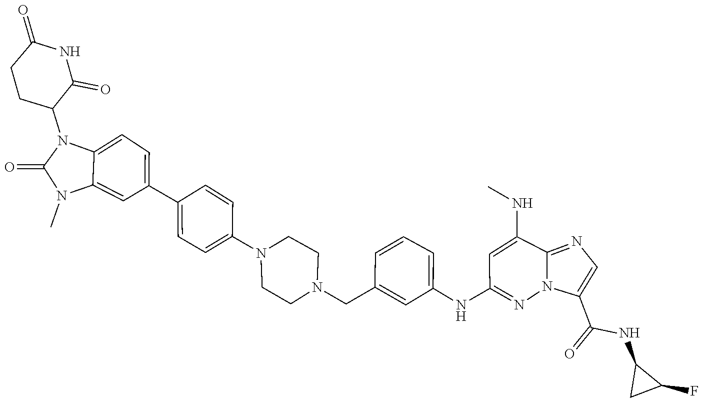 [SMILES: CNc1cc(Nc2cccc(CN3CCN(c4ccc(-c5ccc6c(c5)n(C)c(=O)n6C5CCC(=O)NC5=O)cc4)CC3)c2)nn2c(C(=O)N[C@@H]3C[C@@H]3F)cnc12] |
| K487 | 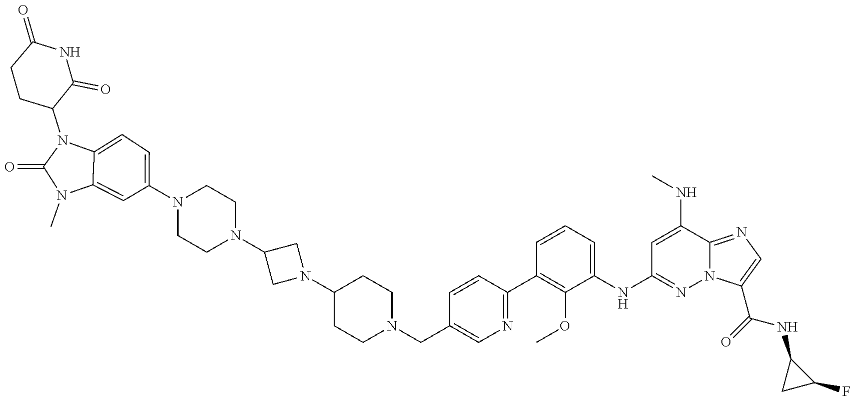 [SMILES: CNc1cc(Nc2cccc(-c3ccc(CN4CCC(N5CC(N6CCN(c7ccc8c(c7)n(C)c(=O)n8C7CCC(=O)NC7=O)CC6)C5)CC4)cn3)c2OC)nn2c(C(=O)N[C@@H]3C[C@@H]3F)cnc12] |
| K488 | 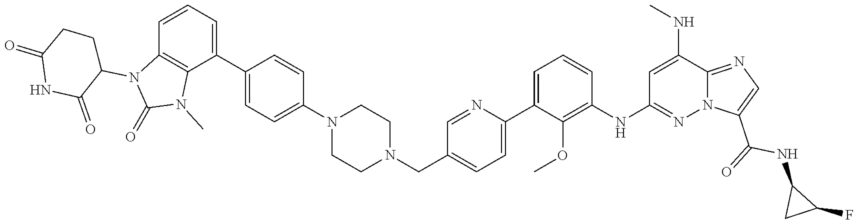 [SMILES: CNc1cc(Nc2cccc(-c3ccc(CN4CCN(c5ccc(-c6cccc7c6n(C)c(=O)n7C6CCC(=O)NC6=O)cc5)CC4)cn3)c2OC)nn2c(C(=O)N[C@@H]3C[C@@H]3F)cnc12] |
| K489 | 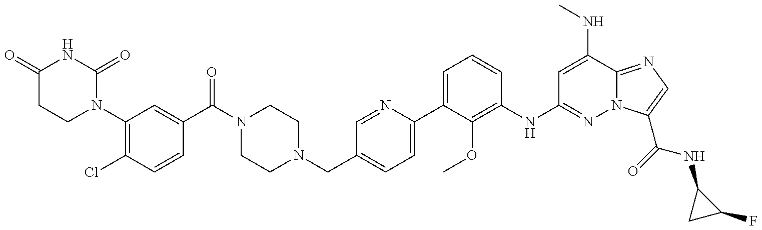 [SMILES: CNc1cc(Nc2cccc(-c3ccc(CN4CCN(C(=O)c5ccc(Cl)c(N6CCC(=O)NC6=O)c5)CC4)cn3)c2OC)nn2c(C(=O)N[C@@H]3C[C@@H]3F)cnc12] |
| K490 | 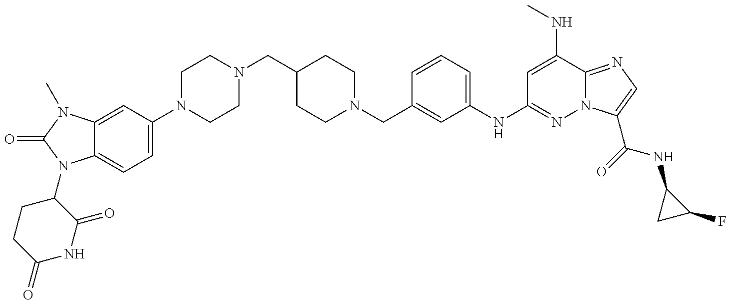 [SMILES: CNc1cc(Nc2cccc(CN3CCC(CN4CCN(c5ccc6c(c5)n(C)c(=O)n6C5CCC(=O)NC5=O)CC4)CC3)c2)nn2c(C(=O)N[C@@H]3C[C@@H]3F)cnc12] |
| K491 | 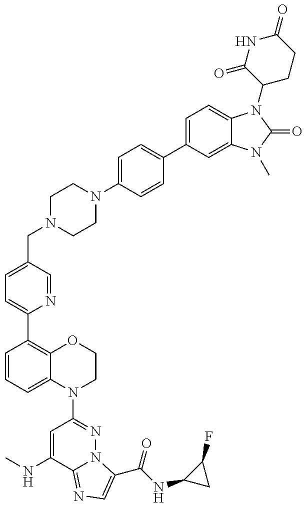 [SMILES: CNc1cc(N2CCOc3c(-c4ccc(CN5CCN(c6ccc(-c7ccc8c(c7)n(C)c(=O)n8C7CCC(=O)NC7=O)cc6)CC5)cn4)cccc32)nn2c(C(=O)N[C@@H]3C[C@@H]3F)cnc12] |
| K492 | 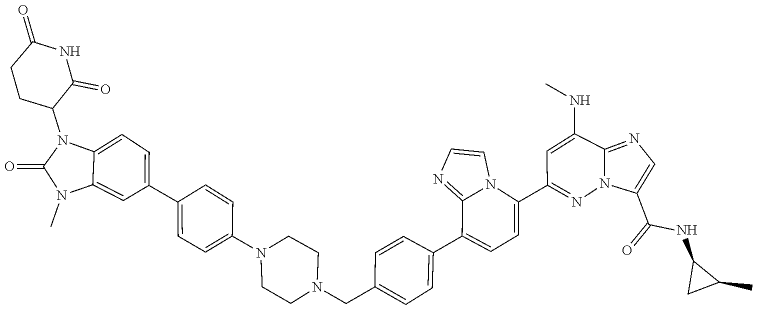 [SMILES: CNc1cc(-c2ccc(-c3ccc(CN4CCN(c5ccc(-c6ccc7c(c6)n(C)c(=O)n7C6CCC(=O)NC6=O)cc5)CC4)cc3)c3nccn23)nn2c(C(=O)N[C@@H]3C[C@@H]3C)cnc12] |
| K493 | 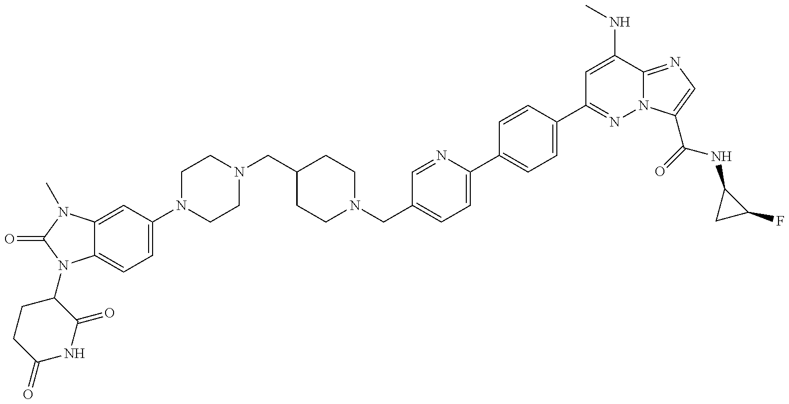 [SMILES: CNc1cc(-c2ccc(-c3ccc(CN4CCC(CN5CCN(c6ccc7c(c6)n(C)c(=O)n7C6CCC(=O)NC6=O)CC5)CC4)cn3)cc2)nn2c(C(=O)N[C@@H]3C[C@@H]3F)cnc12] |
| K494 | 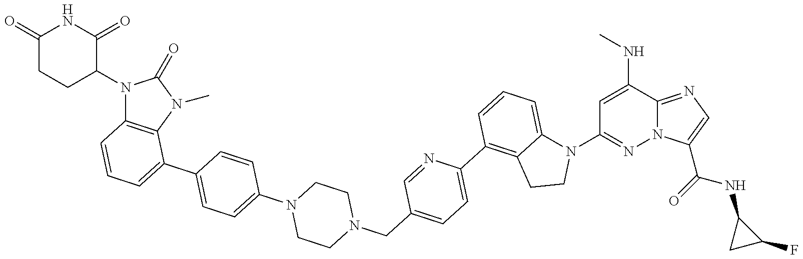 [SMILES: CNc1cc(N2CCc3c(-c4ccc(CN5CCN(c6ccc(-c7cccc8c7n(C)c(=O)n8C7CCC(=O)NC7=O)cc6)CC5)cn4)cccc32)nn2c(C(=O)N[C@@H]3C[C@@H]3F)cnc12] |
| K495 | 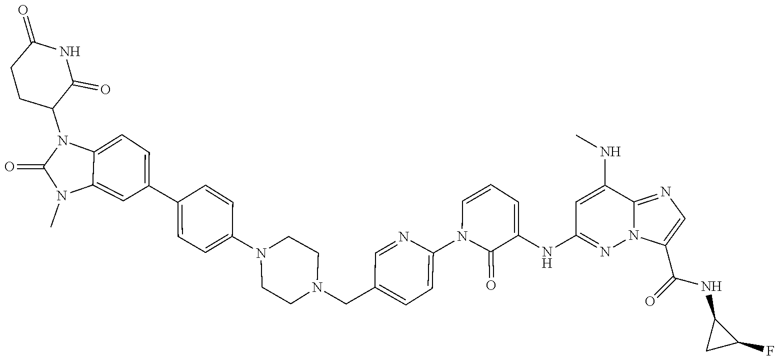 [SMILES: CNc1cc(Nc2cccn(-c3ccc(CN4CCN(c5ccc(-c6ccc7c(c6)n(C)c(=O)n7C6CCC(=O)NC6=O)cc5)CC4)cn3)c2=O)nn2c(C(=O)N[C@@H]3C[C@@H]3F)cnc12] |
| K496 | 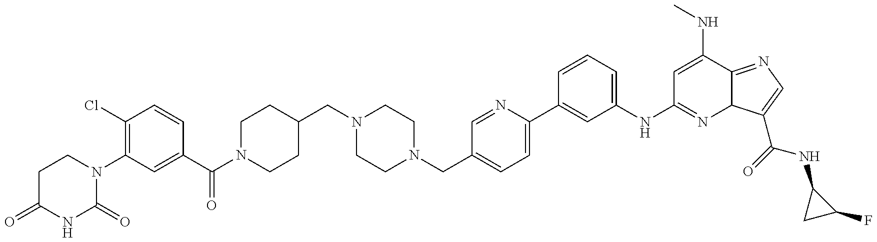 [SMILES: CNC1=CC(Nc2cccc(-c3ccc(CN4CCN(CC5CCN(C(=O)c6ccc(Cl)c(N7CCC(=O)NC7=O)c6)CC5)CC4)cn3)c2)=NC2C(C(=O)N[C@@H]3C[C@@H]3F)=CN=C12] |
| K497 | 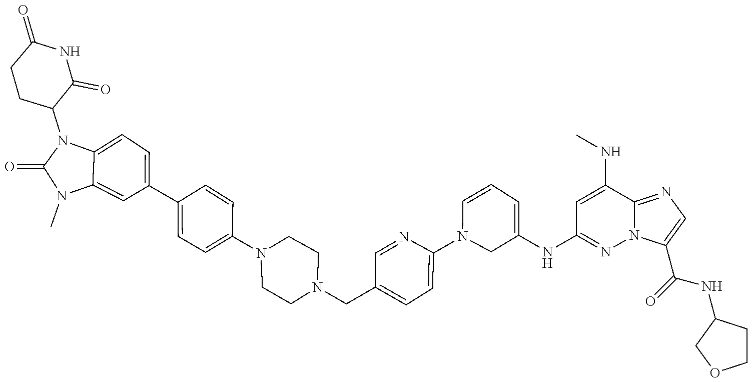 [SMILES: CNc1cc(NC2=CC=CN(c3ccc(CN4CCN(c5ccc(-c6ccc7c(c6)n(C)c(=O)n7C6CCC(=O)NC6=O)cc5)CC4)cn3)C2)nn2c(C(=O)NC3CCOC3)cnc12] |
| K498 | 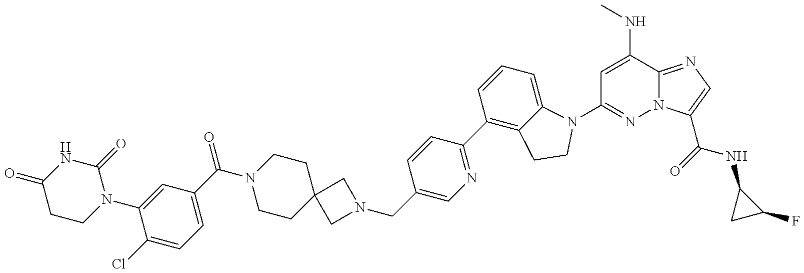 [SMILES: CNc1cc(N2CCc3c(-c4ccc(CN5CC6(CCN(C(=O)c7ccc(Cl)c(N8CCC(=O)NC8=O)c7)CC6)C5)cn4)cccc32)nn2c(C(=O)N[C@@H]3C[C@@H]3F)cnc12] |
| K499 | 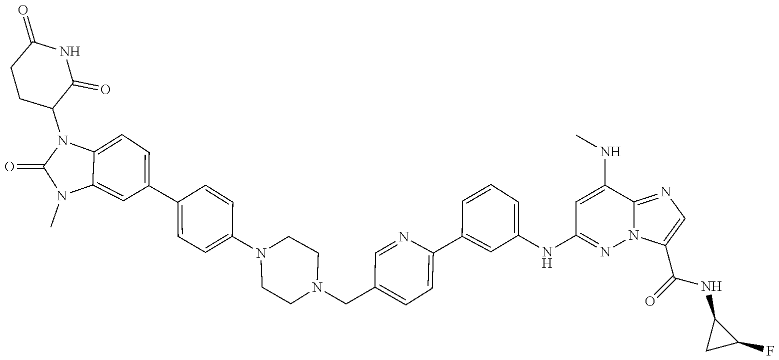 [SMILES: CNc1cc(Nc2cccc(-c3ccc(CN4CCN(c5ccc(-c6ccc7c(c6)n(C)c(=O)n7C6CCC(=O)NC6=O)cc5)CC4)cn3)c2)nn2c(C(=O)N[C@@H]3C[C@@H]3F)cnc12] |
| K500 | 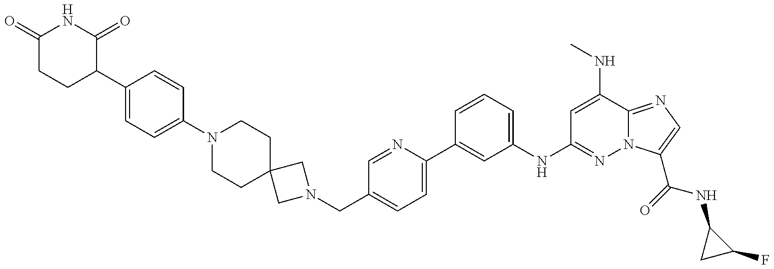 [SMILES: CNc1cc(Nc2cccc(-c3ccc(CN4CC5(CCN(c6ccc(C7CCC(=O)NC7=O)cc6)CC5)C4)cn3)c2)nn2c(C(=O)N[C@@H]3C[C@@H]3F)cnc12] |
| K501 | 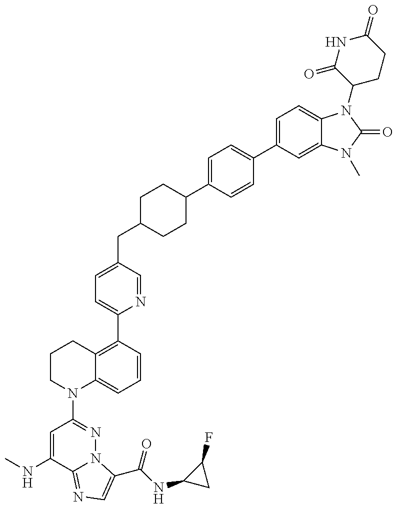 [SMILES: CNc1cc(N2CCCc3c(-c4ccc(CC5CCC(c6ccc(-c7ccc8c(c7)n(C)c(=O)n8C7CCC(=O)NC7=O)cc6)CC5)cn4)cccc32)nn2c(C(=O)N[C@@H]3C[C@@H]3F)cnc12] |
| K502 | 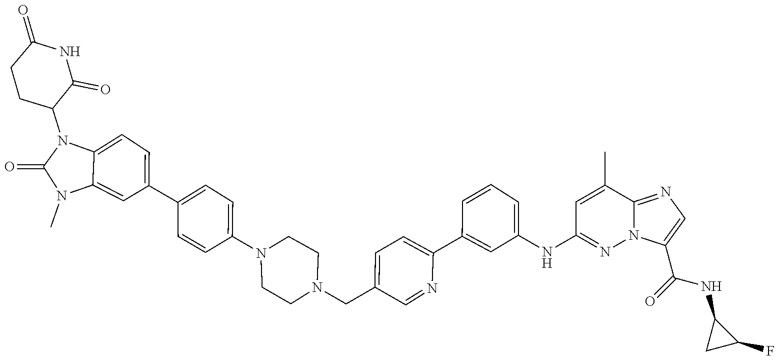 [SMILES: Cc1cc(Nc2cccc(-c3ccc(CN4CCN(c5ccc(-c6ccc7c(c6)n(C)c(=O)n7C6CCC(=O)NC6=O)cc5)CC4)cn3)c2)nn2c(C(=O)N[C@@H]3C[C@@H]3F)cnc12] |
| K503 | 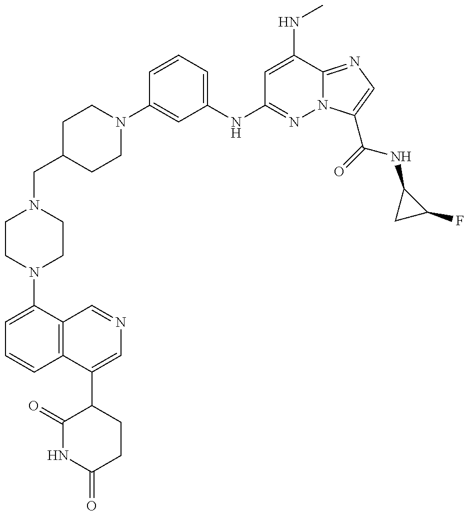 [SMILES: CNc1cc(Nc2cccc(N3CCC(CN4CCN(c5cccc6c(C7CCC(=O)NC7=O)cncc56)CC4)CC3)c2)nn2c(C(=O)N[C@@H]3C[C@@H]3F)cnc12] |
| K504 | 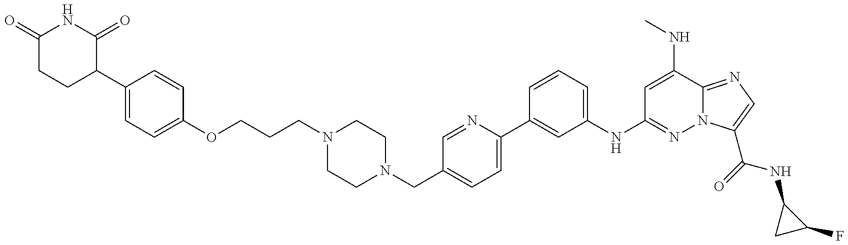 [SMILES: CNc1cc(Nc2cccc(-c3ccc(CN4CCN(CCCOc5ccc(C6CCC(=O)NC6=O)cc5)CC4)cn3)c2)nn2c(C(=O)N[C@@H]3C[C@@H]3F)cnc12] |
| K505 | 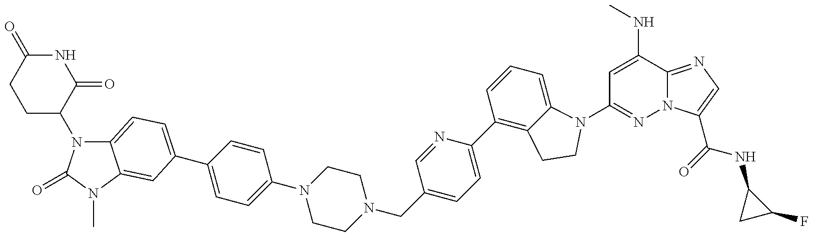 [SMILES: CNc1cc(N2CCc3c(-c4ccc(CN5CCN(c6ccc(-c7ccc8c(c7)n(C)c(=O)n8C7CCC(=O)NC7=O)cc6)CC5)cn4)cccc32)nn2c(C(=O)N[C@@H]3C[C@@H]3F)cnc12] |
| K506 | 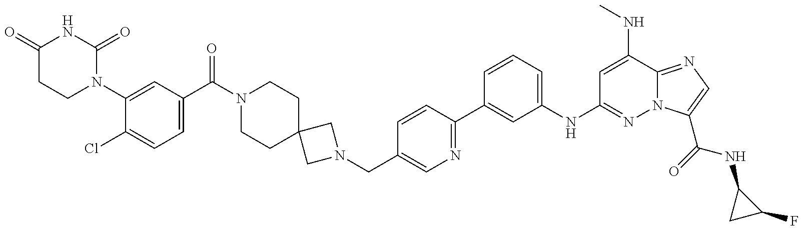 [SMILES: CNc1cc(Nc2cccc(-c3ccc(CN4CC5(CCN(C(=O)c6ccc(Cl)c(N7CCC(=O)NC7=O)c6)CC5)C4)cn3)c2)nn2c(C(=O)N[C@@H]3C[C@@H]3F)cnc12] |
| K507 | 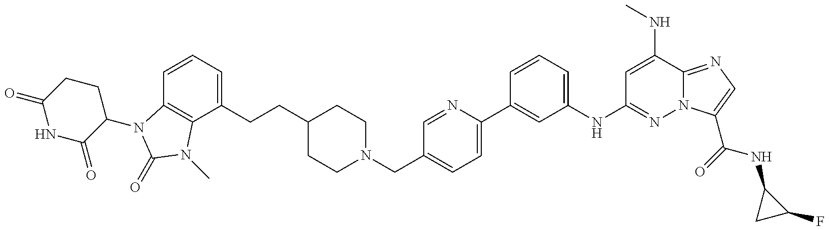 [SMILES: CNc1cc(Nc2cccc(-c3ccc(CN4CCC(CCc5cccc6c5n(C)c(=O)n6C5CCC(=O)NC5=O)CC4)cn3)c2)nn2c(C(=O)N[C@@H]3C[C@@H]3F)cnc12] |
| K508 | 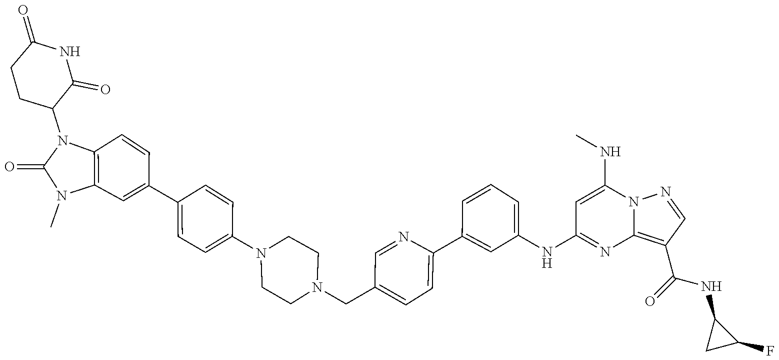 [SMILES: CNc1cc(Nc2cccc(-c3ccc(CN4CCN(c5ccc(-c6ccc7c(c6)n(C)c(=O)n7C6CCC(=O)NC6=O)cc5)CC4)cn3)c2)nc2c(C(=O)N[C@@H]3C[C@@H]3F)cnn12] |
| K509 | 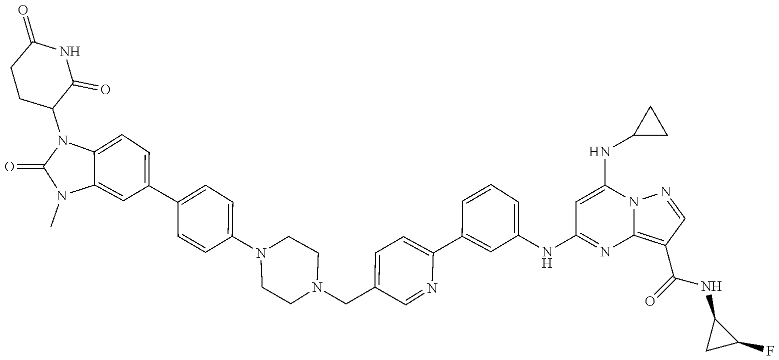 [SMILES: Cn1c(=O)n(C2CCC(=O)NC2=O)c2ccc(-c3ccc(N4CCN(Cc5ccc(-c6cccc(Nc7cc(NC8CC8)n8ncc(C(=O)N[C@@H]9C[C@@H]9F)c8n7)c6)nc5)CC4)cc3)cc21] |
| K510 | 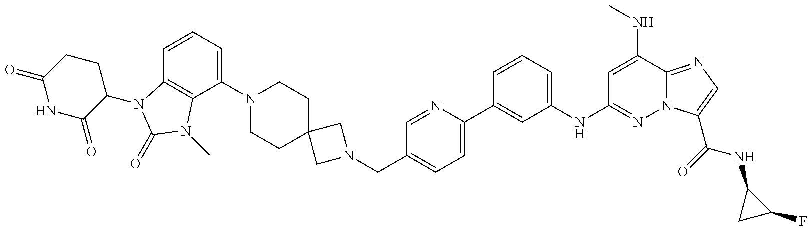 [SMILES: CNc1cc(Nc2cccc(-c3ccc(CN4CC5(CCN(c6cccc7c6n(C)c(=O)n7C6CCC(=O)NC6=O)CC5)C4)cn3)c2)nn2c(C(=O)N[C@@H]3C[C@@H]3F)cnc12] |
| K511 | 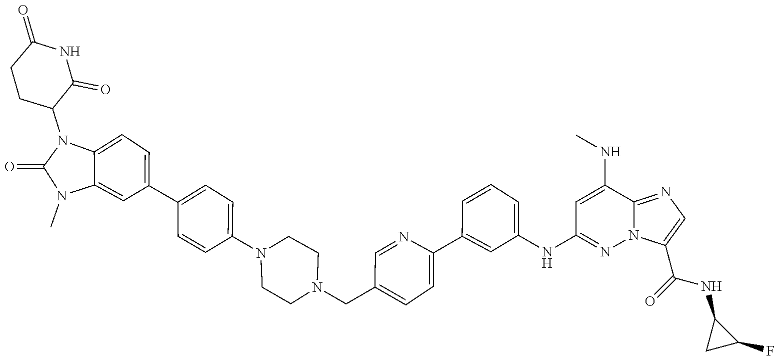 [SMILES: CNc1cc(Nc2cccc(-c3ccc(CN4CCN(c5ccc(-c6ccc7c(c6)n(C)c(=O)n7C6CCC(=O)NC6=O)cc5)CC4)cn3)c2)nn2c(C(=O)N[C@@H]3C[C@@H]3F)cnc12] |
| K512 | 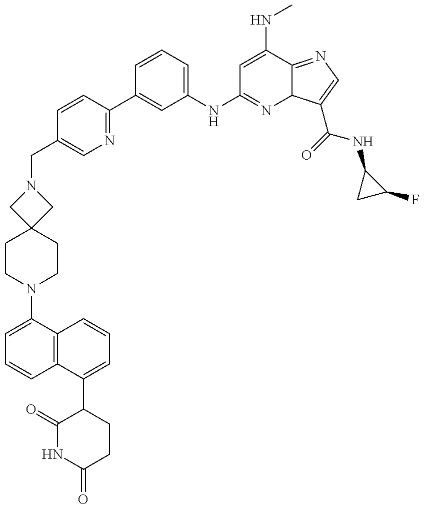 [SMILES: CNC1=CC(Nc2cccc(-c3ccc(CN4CC5(CCN(c6cccc7c(C8CCC(=O)NC8=O)cccc67)CC5)C4)cn3)c2)=NC2C(C(=O)N[C@@H]3C[C@@H]3F)=CN=C12] |
| K513 | 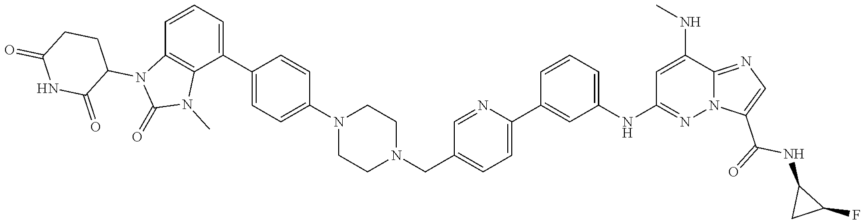 [SMILES: CNc1cc(Nc2cccc(-c3ccc(CN4CCN(c5ccc(-c6cccc7c6n(C)c(=O)n7C6CCC(=O)NC6=O)cc5)CC4)cn3)c2)nn2c(C(=O)N[C@@H]3C[C@@H]3F)cnc12] |
| K514 | 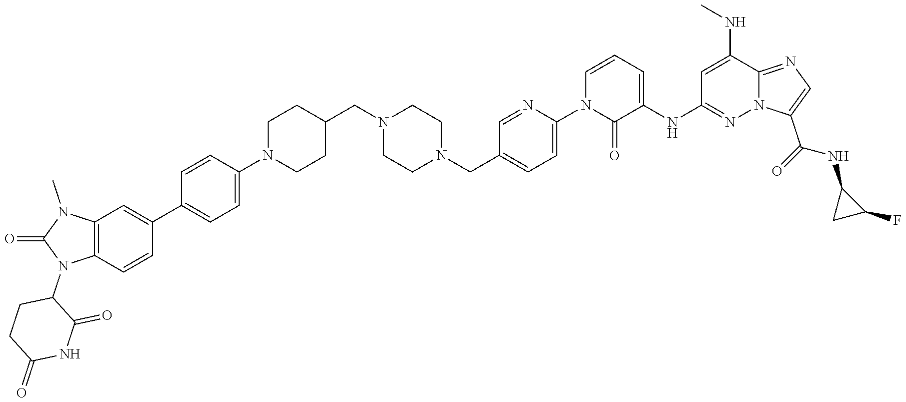 [SMILES: CNc1cc(Nc2cccn(-c3ccc(CN4CCN(CC5CCN(c6ccc(-c7ccc8c(c7)n(C)c(=O)n8C7CCC(=O)NC7=O)cc6)CC5)CC4)cn3)c2=O)nn2c(C(=O)N[C@@H]3C[C@@H]3F)cnc12] |
| K515 | 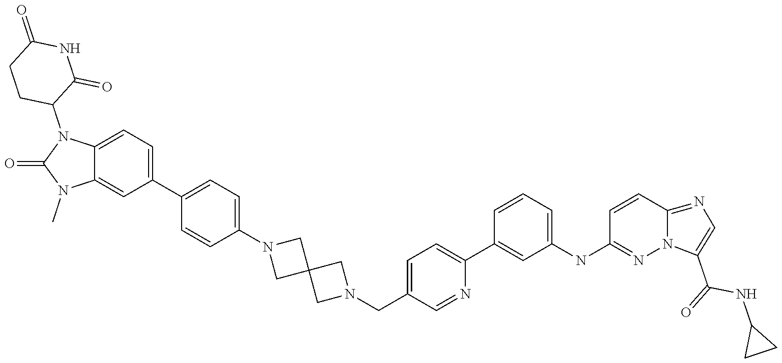 |
| K516 | 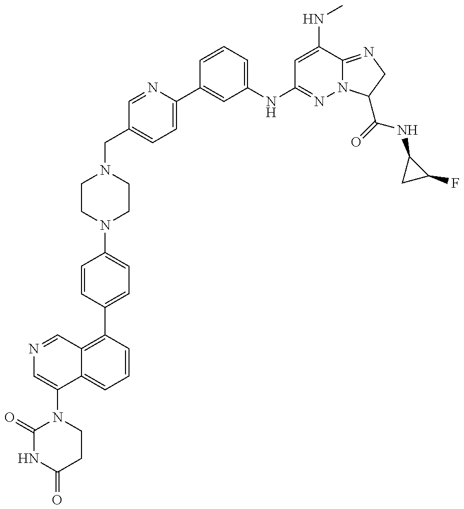 [SMILES: CNC1=CC(Nc2cccc(-c3ccc(CN4CCN(c5ccc(-c6cccc7c(N8CCC(=O)NC8=O)cncc67)cc5)CC4)cn3)c2)=NN2C1=NCC2C(=O)N[C@@H]1C[C@@H]1F] |
| K517 | 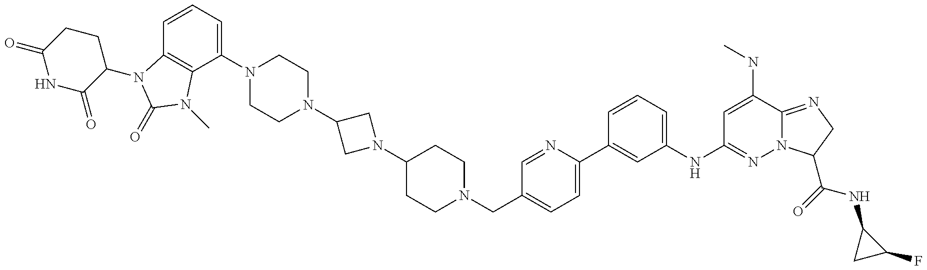 [SMILES: CNC1=CC(Nc2cccc(-c3ccc(CN4CCC(N5CC(N6CCN(c7cccc8c7n(C)c(=O)n8C7CCC(=O)NC7=O)CC6)C5)CC4)cn3)c2)=NN2C1=NCC2C(=O)N[C@@H]1C[C@@H]1F] |
| K518 | 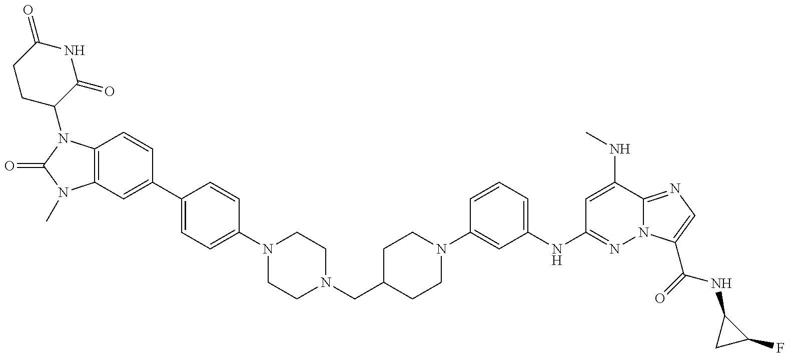 [SMILES: CNc1cc(Nc2cccc(N3CCC(CN4CCN(c5ccc(-c6ccc7c(c6)n(C)c(=O)n7C6CCC(=O)NC6=O)cc5)CC4)CC3)c2)nn2c(C(=O)N[C@@H]3C[C@@H]3F)cnc12] |
| K519 | 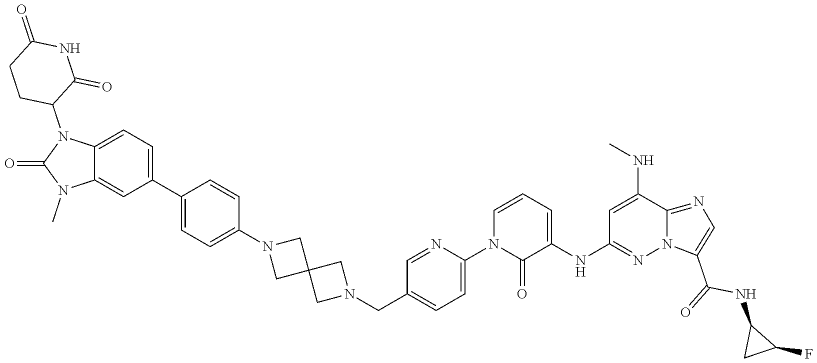 [SMILES: CNc1cc(Nc2cccn(-c3ccc(CN4CC5(C4)CN(c4ccc(-c6ccc7c(c6)n(C)c(=O)n7C6CCC(=O)NC6=O)cc4)C5)cn3)c2=O)nn2c(C(=O)N[C@@H]3C[C@@H]3F)cnc12] |
| K520 | 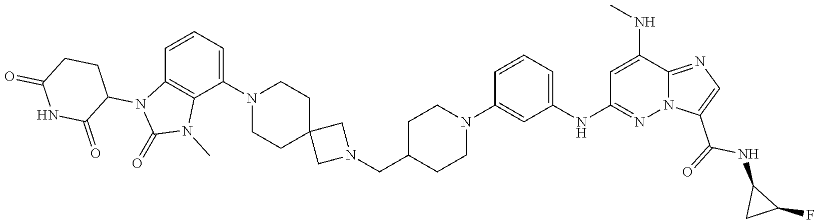 [SMILES: CNc1cc(Nc2cccc(N3CCC(CN4CC5(CCN(c6cccc7c6n(C)c(=O)n7C6CCC(=O)NC6=O)CC5)C4)CC3)c2)nn2c(C(=O)N[C@@H]3C[C@@H]3F)cnc12] |
| K521 | 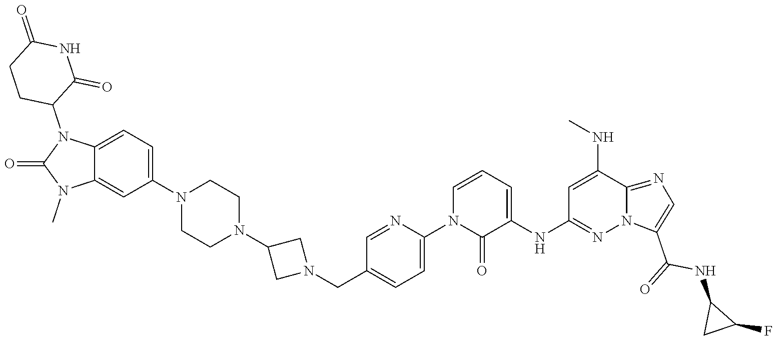 [SMILES: CNc1cc(Nc2cccn(-c3ccc(CN4CC(N5CCN(c6ccc7c(c6)n(C)c(=O)n7C6CCC(=O)NC6=O)CC5)C4)cn3)c2=O)nn2c(C(=O)N[C@@H]3C[C@@H]3F)cnc12] |
| K522 | 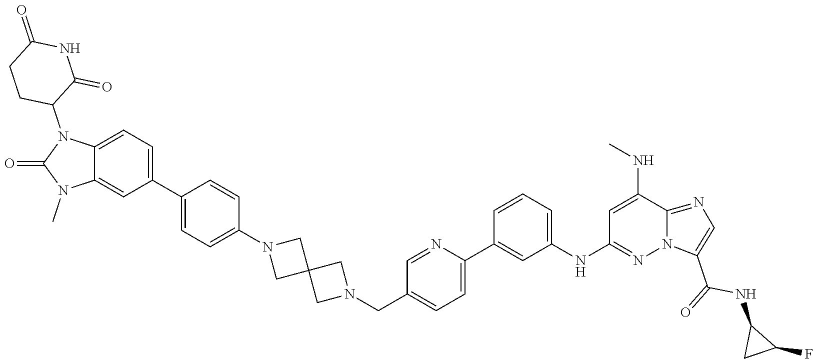 [SMILES: CNc1cc(Nc2cccc(-c3ccc(CN4CC5(C4)CN(c4ccc(-c6ccc7c(c6)n(C)c(=O)n7C6CCC(=O)NC6=O)cc4)C5)cn3)c2)nn2c(C(=O)N[C@@H]3C[C@@H]3F)cnc12] |
| K523 | 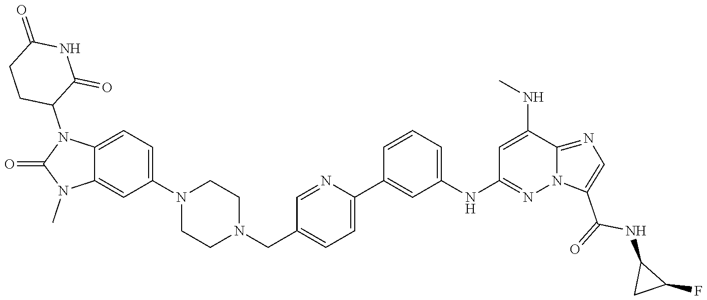 [SMILES: CNc1cc(Nc2cccc(-c3ccc(CN4CCN(c5ccc6c(c5)n(C)c(=O)n6C5CCC(=O)NC5=O)CC4)cn3)c2)nn2c(C(=O)N[C@@H]3C[C@@H]3F)cnc12] |
| K524 | 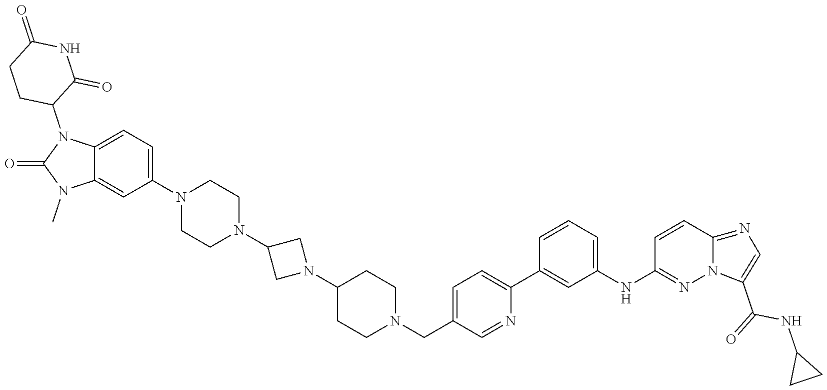 [SMILES: Cn1c(=O)n(C2CCC(=O)NC2=O)c2ccc(N3CCN(C4CN(C5CCN(Cc6ccc(-c7cccc(Nc8ccc9ncc(C(=O)NC%10CC%10)n9n8)c7)nc6)CC5)C4)CC3)cc21] |
| K525 | 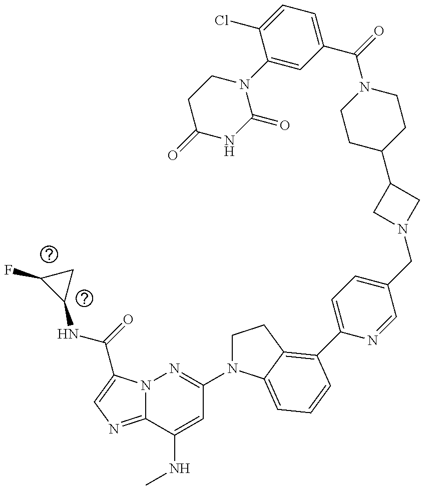 [SMILES: CNc1cc(N2CCc3c(-c4ccc(CN5CC(C6CCN(C(=O)c7ccc(Cl)c(N8CCC(=O)NC8=O)c7)CC6)C5)cn4)cccc32)nn2c(C(=O)N[C@@H]3C[C@@H]3F)cnc12] |
| K526 | 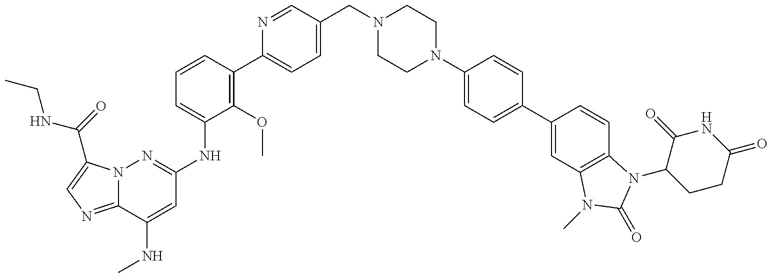 [SMILES: CCNC(=O)c1cnc2c(NC)cc(Nc3cccc(-c4ccc(CN5CCN(c6ccc(-c7ccc8c(c7)n(C)c(=O)n8C7CCC(=O)NC7=O)cc6)CC5)cn4)c3OC)nn12] |
| K527 | 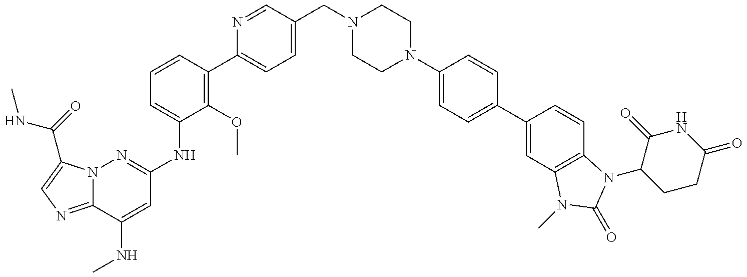 [SMILES: CNC(=O)c1cnc2c(NC)cc(Nc3cccc(-c4ccc(CN5CCN(c6ccc(-c7ccc8c(c7)n(C)c(=O)n8C7CCC(=O)NC7=O)cc6)CC5)cn4)c3OC)nn12] |
| K528 | 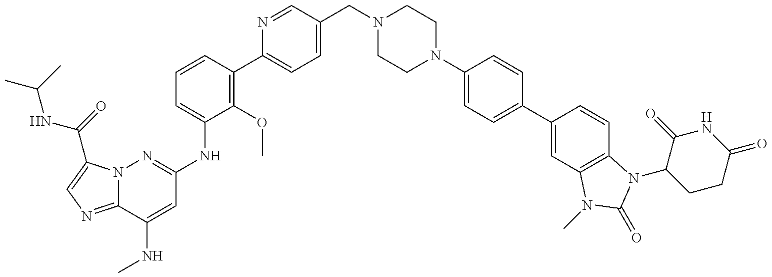 [SMILES: CNc1cc(Nc2cccc(-c3ccc(CN4CCN(c5ccc(-c6ccc7c(c6)n(C)c(=O)n7C6CCC(=O)NC6=O)cc5)CC4)cn3)c2OC)nn2c(C(=O)NC(C)C)cnc12] |
| K529 | 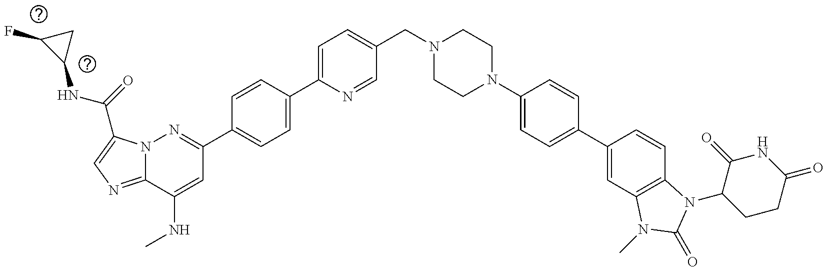 [SMILES: CNc1cc(-c2ccc(-c3ccc(CN4CCN(c5ccc(-c6ccc7c(c6)n(C)c(=O)n7C6CCC(=O)NC6=O)cc5)CC4)cn3)cc2)nn2c(C(=O)N[C@@H]3C[C@@H]3F)cnc12] |
| K530 | 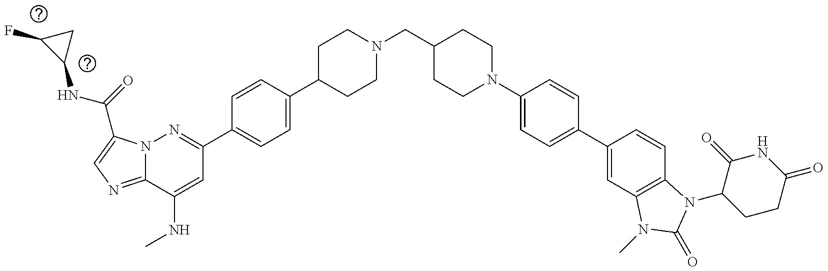 [SMILES: CNc1cc(-c2ccc(C3CCN(CC4CCN(c5ccc(-c6ccc7c(c6)n(C)c(=O)n7C6CCC(=O)NC6=O)cc5)CC4)CC3)cc2)nn2c(C(=O)N[C@@H]3C[C@@H]3F)cnc12] |
| K531 | 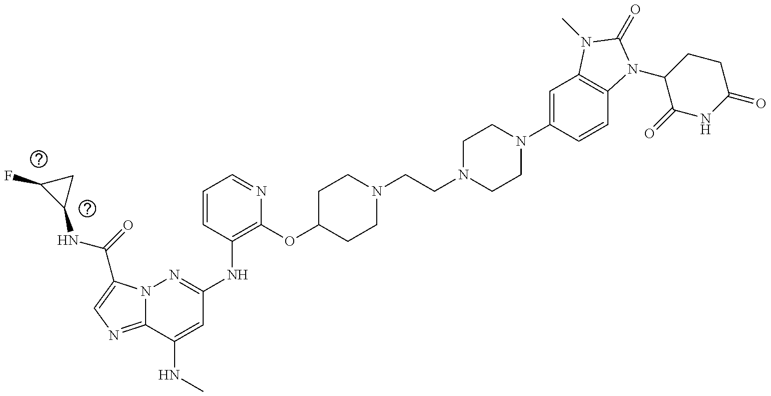 [SMILES: CNc1cc(Nc2cccnc2OC2CCN(CCN3CCN(c4ccc5c(c4)n(C)c(=O)n5C4CCC(=O)NC4=O)CC3)CC2)nn2c(C(=O)N[C@@H]3C[C@@H]3F)cnc12] |
| K532 | 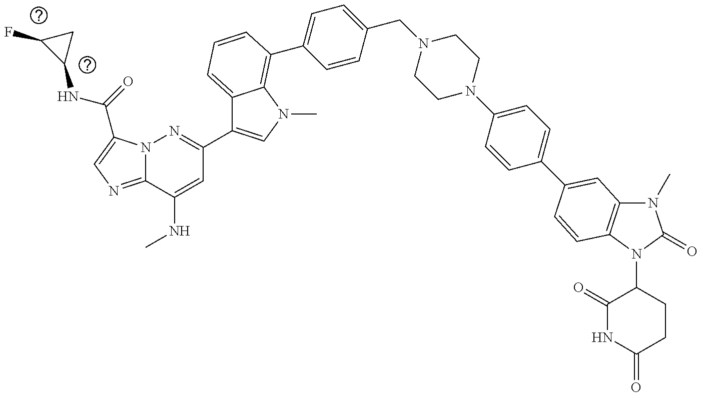 [SMILES: CNc1cc(-c2cn(C)c3c(-c4ccc(CN5CCN(c6ccc(-c7ccc8c(c7)n(C)c(=O)n8C7CCC(=O)NC7=O)cc6)CC5)cc4)cccc23)nn2c(C(=O)N[C@@H]3C[C@@H]3F)cnc12] |
| K533 | 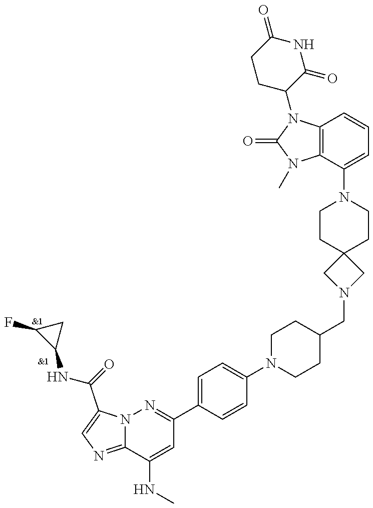 [SMILES: CNc1cc(-c2ccc(N3CCC(CN4CC5(CCN(c6cccc7c6n(C)c(=O)n7C6CCC(=O)NC6=O)CC5)C4)CC3)cc2)nn2c(C(=O)N[C@@H]3C[C@@H]3F)cnc12] |
| K534 | 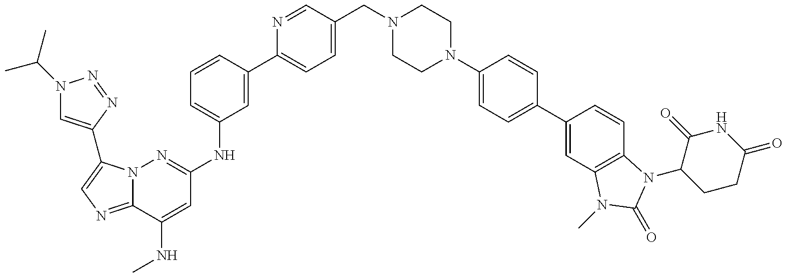 [SMILES: CNc1cc(Nc2cccc(-c3ccc(CN4CCN(c5ccc(-c6ccc7c(c6)n(C)c(=O)n7C6CCC(=O)NC6=O)cc5)CC4)cn3)c2)nn2c(-c3cn(C(C)C)nn3)cnc12] |
| K535 | 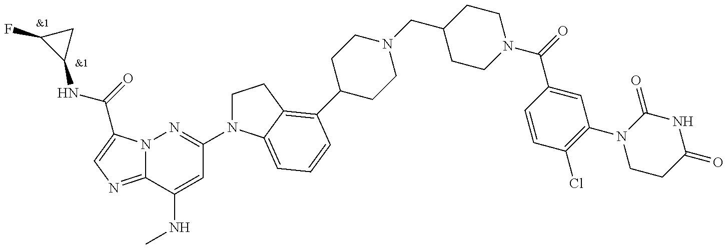 [SMILES: CNc1cc(N2CCc3c(C4CCN(CC5CCN(C(=O)c6ccc(Cl)c(N7CCC(=O)NC7=O)c6)CC5)CC4)cccc32)nn2c(C(=O)N[C@@H]3C[C@@H]3F)cnc12] |
| K536 | 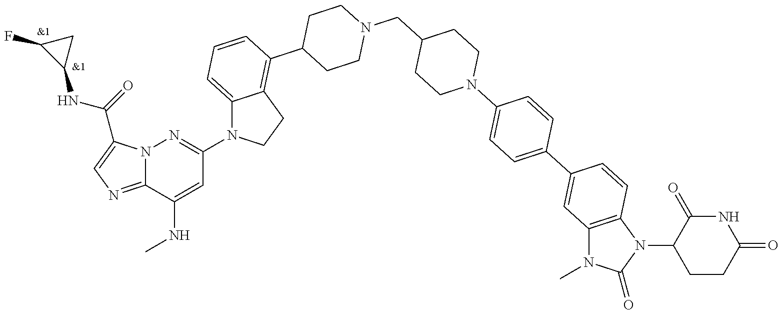 [SMILES: CNc1cc(N2CCc3c(C4CCN(CC5CCN(c6ccc(-c7ccc8c(c7)n(C)c(=O)n8C7CCC(=O)NC7=O)cc6)CC5)CC4)cccc32)nn2c(C(=O)N[C@@H]3C[C@@H]3F)cnc12] |
| K537 | 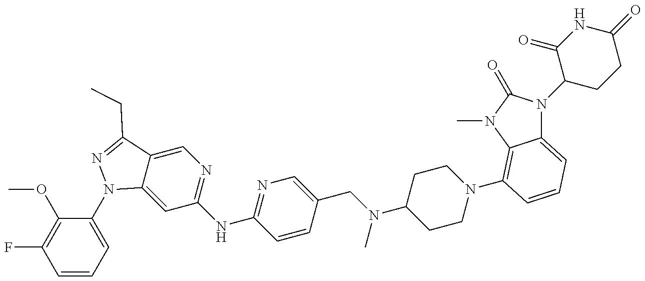 [SMILES: CCc1nn(-c2cccc(F)c2OC)c2cc(Nc3ccc(CN(C)C4CCN(c5cccc6c5n(C)c(=O)n6C5CCC(=O)NC5=O)CC4)cn3)ncc12] |
| K538 | 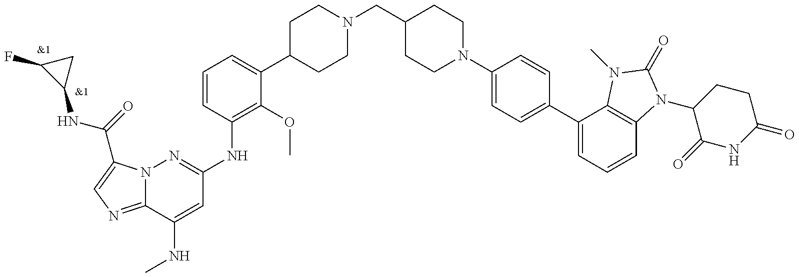 [SMILES: CNc1cc(Nc2cccc(C3CCN(CC4CCN(c5ccc(-c6cccc7c6n(C)c(=O)n7C6CCC(=O)NC6=O)cc5)CC4)CC3)c2OC)nn2c(C(=O)N[C@@H]3C[C@@H]3F)cnc12] |
| K539 | 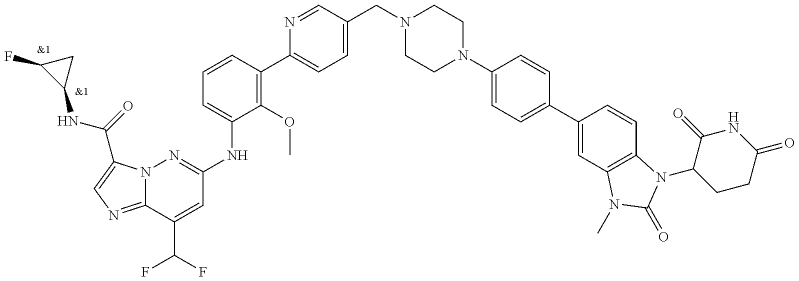 [SMILES: COc1c(Nc2cc(C(F)F)c3ncc(C(=O)N[C@@H]4C[C@@H]4F)n3n2)cccc1-c1ccc(CN2CCN(c3ccc(-c4ccc5c(c4)n(C)c(=O)n5C4CCC(=O)NC4=O)cc3)CC2)cn1] |
| K540 | 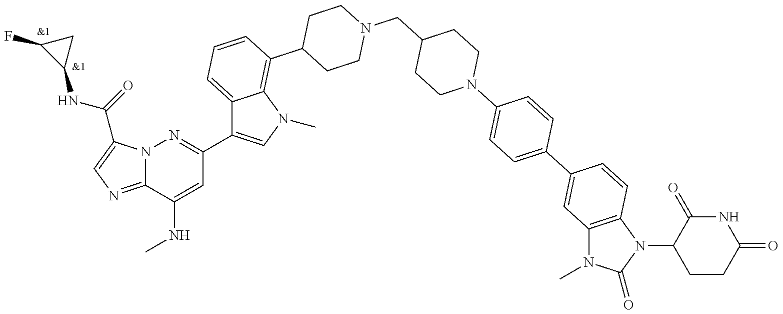 [SMILES: CNc1cc(-c2cn(C)c3c(C4CCN(CC5CCN(c6ccc(-c7ccc8c(c7)n(C)c(=O)n8C7CCC(=O)NC7=O)cc6)CC5)CC4)cccc23)nn2c(C(=O)N[C@@H]3C[C@@H]3F)cnc12] |
| K541 | 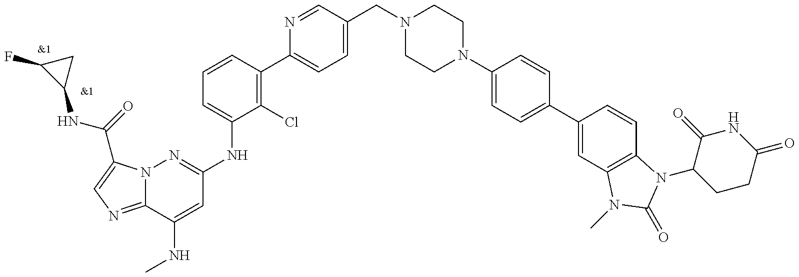 [SMILES: CNc1cc(Nc2cccc(-c3ccc(CN4CCN(c5ccc(-c6ccc7c(c6)n(C)c(=O)n7C6CCC(=O)NC6=O)cc5)CC4)cn3)c2Cl)nn2c(C(=O)N[C@@H]3C[C@@H]3F)cnc12] |
| K542 | 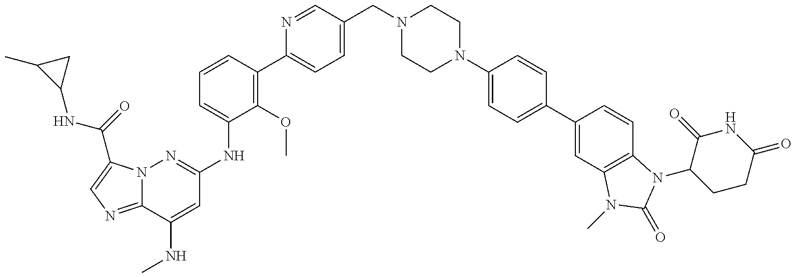 [SMILES: CNc1cc(Nc2cccc(-c3ccc(CN4CCN(c5ccc(-c6ccc7c(c6)n(C)c(=O)n7C6CCC(=O)NC6=O)cc5)CC4)cn3)c2OC)nn2c(C(=O)NC3CC3C)cnc12] |
| K543 | 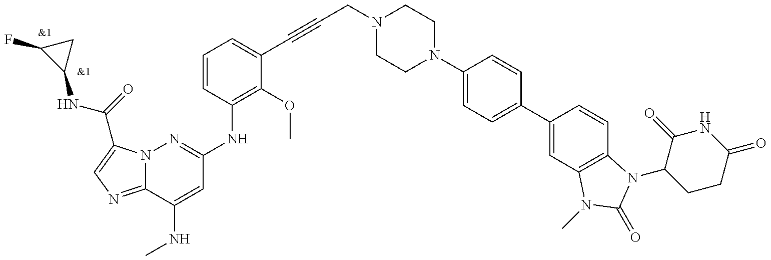 [SMILES: CNc1cc(Nc2cccc(C#CCN3CCN(c4ccc(-c5ccc6c(c5)n(C)c(=O)n6C5CCC(=O)NC5=O)cc4)CC3)c2OC)nn2c(C(=O)N[C@@H]3C[C@@H]3F)cnc12] |
| K544 | 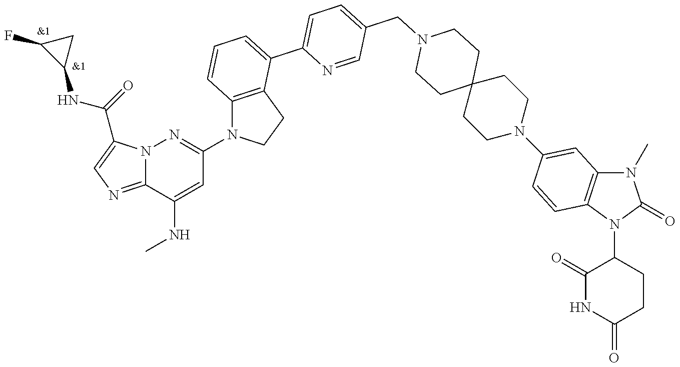 [SMILES: CNc1cc(N2CCc3c(-c4ccc(CN5CCC6(CC5)CCN(c5ccc7c(c5)n(C)c(=O)n7C5CCC(=O)NC5=O)CC6)cn4)cccc32)nn2c(C(=O)N[C@@H]3C[C@@H]3F)cnc12] |
| K545 | 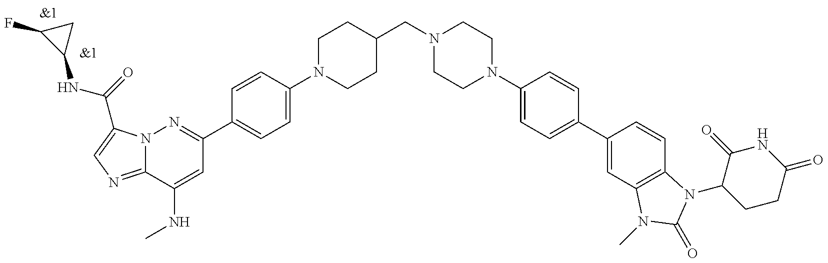 [SMILES: CNc1cc(-c2ccc(N3CCC(CN4CCN(c5ccc(-c6ccc7c(c6)n(C)c(=O)n7C6CCC(=O)NC6=O)cc5)CC4)CC3)cc2)nn2c(C(=O)N[C@@H]3C[C@@H]3F)cnc12] |
| K546 | 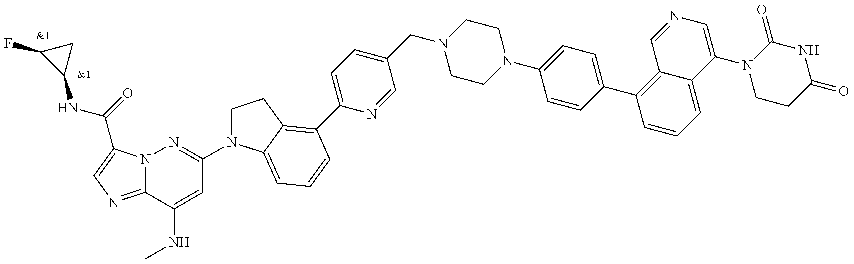 [SMILES: CNc1cc(N2CCc3c(-c4ccc(CN5CCN(c6ccc(-c7cccc8c(N9CCC(=O)NC9=O)cncc78)cc6)CC5)cn4)cccc32)nn2c(C(=O)N[C@@H]3C[C@@H]3F)cnc12] |
| K547 | 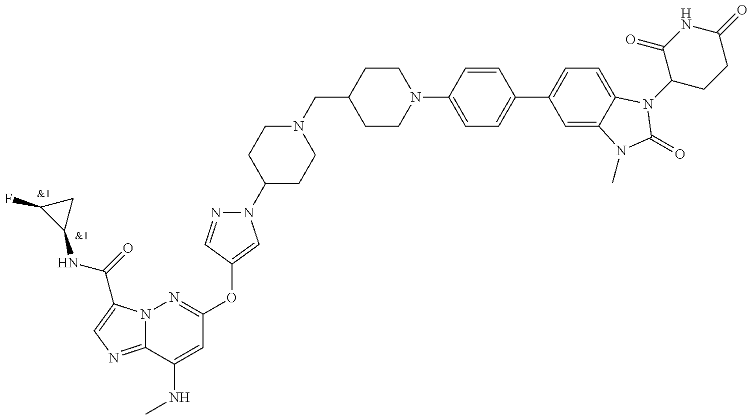 [SMILES: CNc1cc(Oc2cnn(C3CCN(CC4CCN(c5ccc(-c6ccc7c(c6)n(C)c(=O)n7C6CCC(=O)NC6=O)cc5)CC4)CC3)c2)nn2c(C(=O)N[C@@H]3C[C@@H]3F)cnc12] |
| K548 | 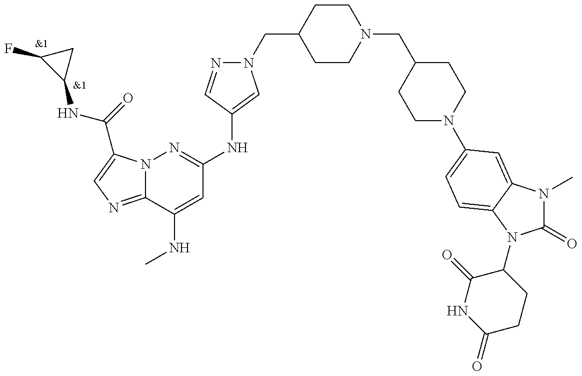 [SMILES: CNc1cc(Nc2cnn(CC3CCN(CC4CCN(c5ccc6c(c5)n(C)c(=O)n6C5CCC(=O)NC5=O)CC4)CC3)c2)nn2c(C(=O)N[C@@H]3C[C@@H]3F)cnc12] |
| K549 | 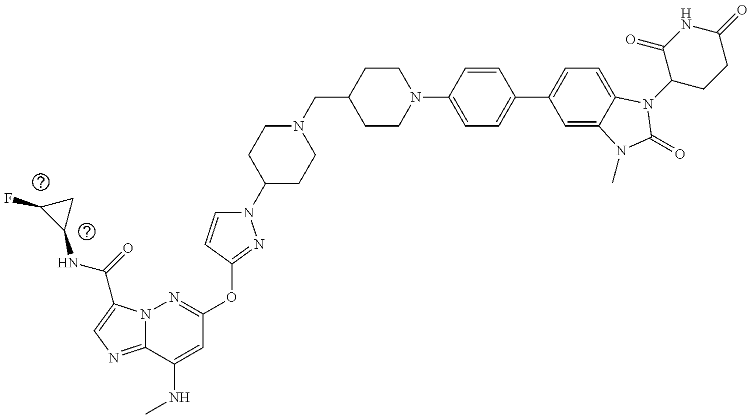 [SMILES: CNc1cc(Oc2ccn(C3CCN(CC4CCN(c5ccc(-c6ccc7c(c6)n(C)c(=O)n7C6CCC(=O)NC6=O)cc5)CC4)CC3)n2)nn2c(C(=O)N[C@@H]3C[C@@H]3F)cnc12] |
| K550 | 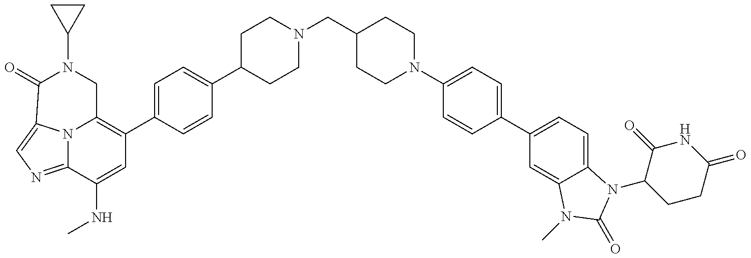 [SMILES: CNc1cc(-c2ccc(C3CCN(CC4CCN(c5ccc(-c6ccc7c(c6)n(C)c(=O)n7C6CCC(=O)NC6=O)cc5)CC4)CC3)cc2)c2n3c(cnc13)C(=O)N(C1CC1)C2] |
| K551 | 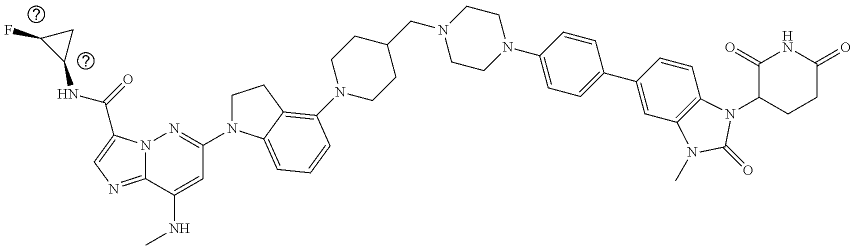 [SMILES: CNc1cc(N2CCc3c(N4CCC(CN5CCN(c6ccc(-c7ccc8c(c7)n(C)c(=O)n8C7CCC(=O)NC7=O)cc6)CC5)CC4)cccc32)nn2c(C(=O)N[C@@H]3C[C@@H]3F)cnc12] |
| K552 | 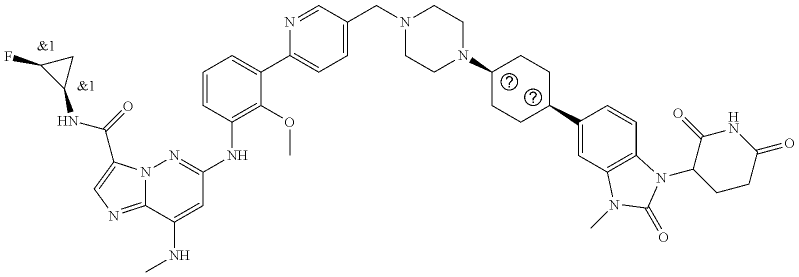 [SMILES: CNc1cc(Nc2cccc(-c3ccc(CN4CCN([C@H]5CC[C@@H](c6ccc7c(c6)n(C)c(=O)n7C6CCC(=O)NC6=O)CC5)CC4)cn3)c2OC)nn2c(C(=O)N[C@@H]3C[C@@H]3F)cnc12] |
| K553 | 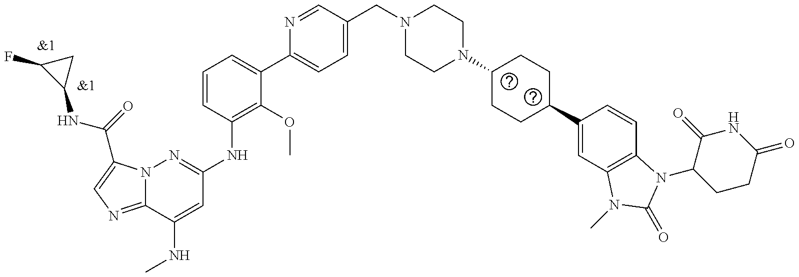 [SMILES: CNc1cc(Nc2cccc(-c3ccc(CN4CCN([C@H]5CC[C@H](c6ccc7c(c6)n(C)c(=O)n7C6CCC(=O)NC6=O)CC5)CC4)cn3)c2OC)nn2c(C(=O)N[C@@H]3C[C@@H]3F)cnc12] |
| K554 | 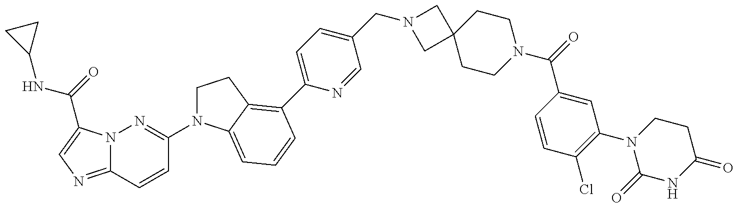 [SMILES: O=C1CCN(c2cc(C(=O)N3CCC4(CC3)CN(Cc3ccc(-c5cccc6c5CCN6c5ccc6ncc(C(=O)NC7CC7)n6n5)nc3)C4)ccc2Cl)C(=O)N1] |
| K555 | 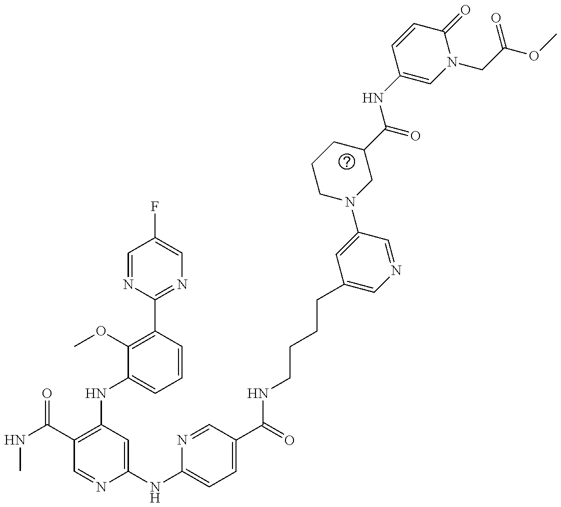 [SMILES: CNC(=O)c1cnc(Nc2ccc(C(=O)NCCCCc3cncc(N4CCCC(C(=O)Nc5ccc(=O)n(CC(=O)OC)c5)C4)c3)cn2)cc1Nc1cccc(-c2ncc(F)cn2)c1OC] |
| K556 | 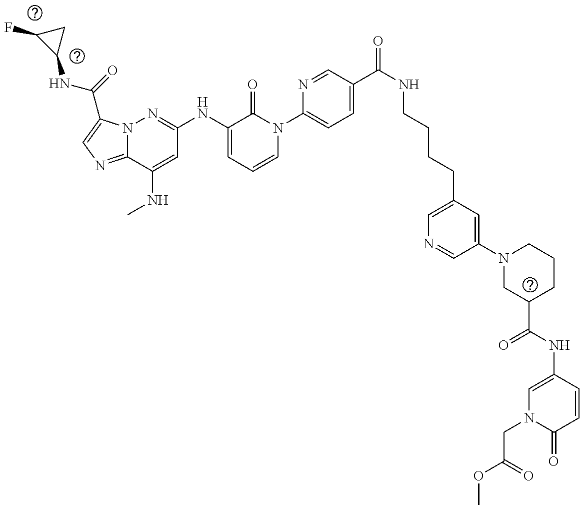 [SMILES: CNc1cc(Nc2cccn(-c3ccc(C(=O)NCCCCc4cncc(N5CCCC(C(=O)Nc6ccc(=O)n(CC(=O)OC)c6)C5)c4)cn3)c2=O)nn2c(C(=O)N[C@@H]3C[C@@H]3F)cnc12] |
| K557 | 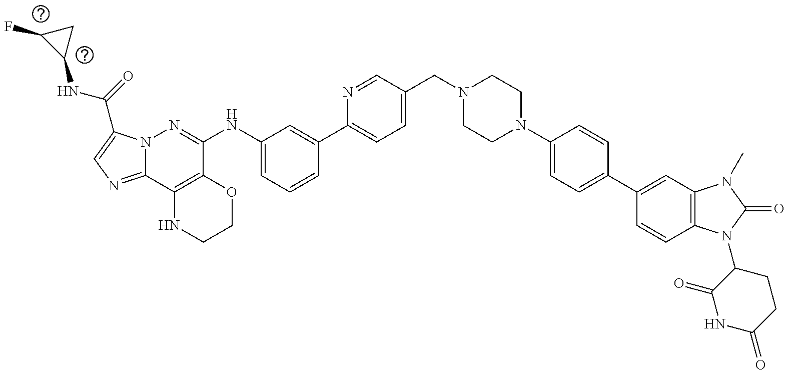 [SMILES: Cn1c(=O)n(C2CCC(=O)NC2=O)c2ccc(-c3ccc(N4CCN(Cc5ccc(-c6cccc(Nc7nn8c(C(=O)N[C@@H]9C[C@@H]9F)cnc8c8c7OCCN8)c6)nc5)CC4)cc3)cc21] |
| K558 | 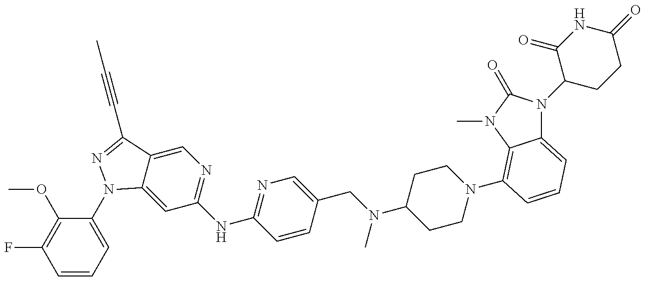 [SMILES: CC#Cc1nn(-c2cccc(F)c2OC)c2cc(Nc3ccc(CN(C)C4CCN(c5cccc6c5n(C)c(=O)n6C5CCC(=O)NC5=O)CC4)cn3)ncc12] |
| K559 | 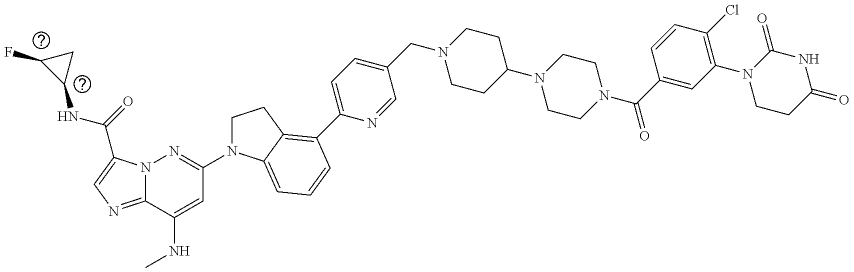 [SMILES: CNc1cc(N2CCc3c(-c4ccc(CN5CCC(N6CCN(C(=O)c7ccc(Cl)c(N8CCC(=O)NC8=O)c7)CC6)CC5)cn4)cccc32)nn2c(C(=O)N[C@@H]3C[C@@H]3F)cnc12] |
| K560 | 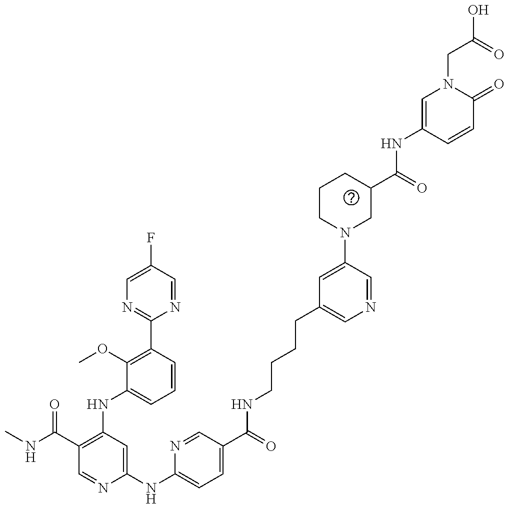 [SMILES: CNC(=O)c1cnc(Nc2ccc(C(=O)NCCCCc3cncc(N4CCCC(C(=O)Nc5ccc(=O)n(CC(=O)O)c5)C4)c3)cn2)cc1Nc1cccc(-c2ncc(F)cn2)c1OC] |
| K561 | 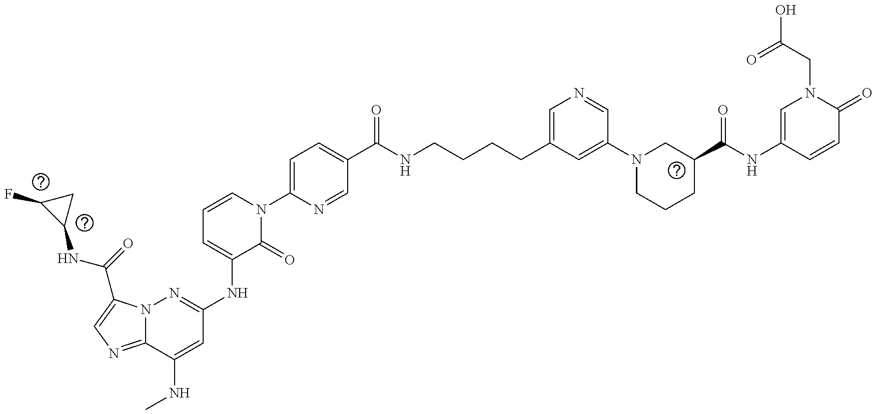 [SMILES: CNc1cc(Nc2cccn(-c3ccc(C(=O)NCCCCc4cncc(N5CCC[C@H](C(=O)Nc6ccc(=O)n(CC(=O)O)c6)C5)c4)cn3)c2=O)nn2c(C(=O)N[C@@H]3C[C@@H]3F)cnc12] |
| K562 | 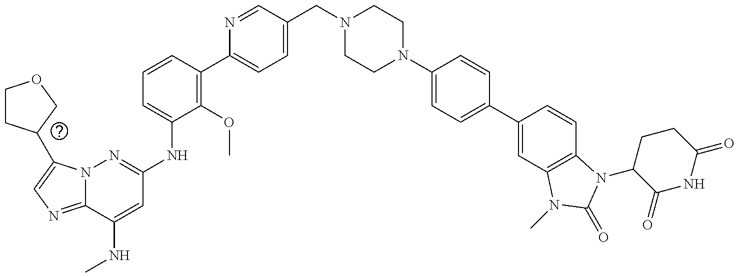 [SMILES: CNc1cc(Nc2cccc(-c3ccc(CN4CCN(c5ccc(-c6ccc7c(c6)n(C)c(=O)n7C6CCC(=O)NC6=O)cc5)CC4)cn3)c2OC)nn2c(C3CCOC3)cnc12] |
| K563 | 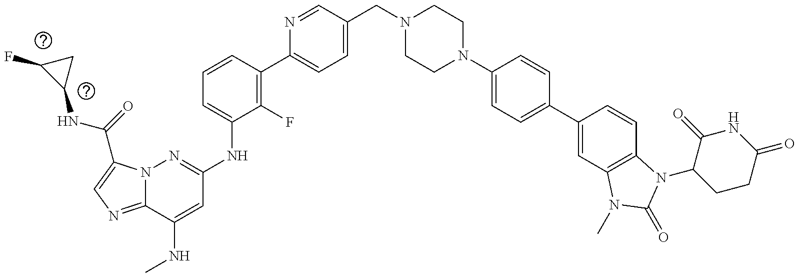 [SMILES: CNc1cc(Nc2cccc(-c3ccc(CN4CCN(c5ccc(-c6ccc7c(c6)n(C)c(=O)n7C6CCC(=O)NC6=O)cc5)CC4)cn3)c2F)nn2c(C(=O)N[C@@H]3C[C@@H]3F)cnc12] |
| K564 | 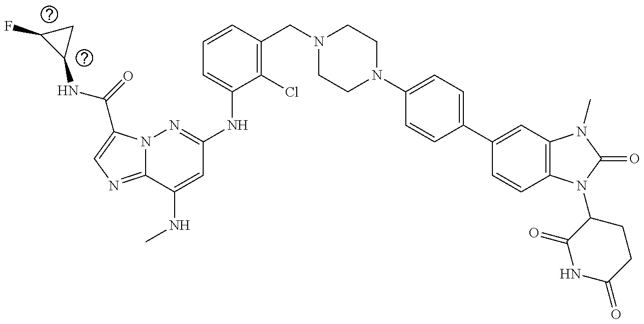 [SMILES: CNc1cc(Nc2cccc(CN3CCN(c4ccc(-c5ccc6c(c5)n(C)c(=O)n6C5CCC(=O)NC5=O)cc4)CC3)c2Cl)nn2c(C(=O)N[C@@H]3C[C@@H]3F)cnc12] |
| K565 | 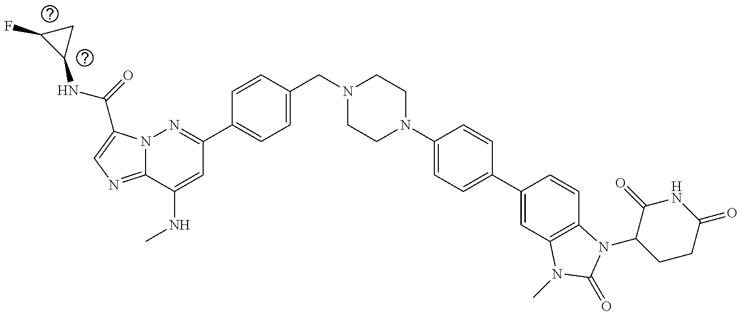 [SMILES: CNc1cc(-c2ccc(CN3CCN(c4ccc(-c5ccc6c(c5)n(C)c(=O)n6C5CCC(=O)NC5=O)cc4)CC3)cc2)nn2c(C(=O)N[C@@H]3C[C@@H]3F)cnc12] |
| K566 | 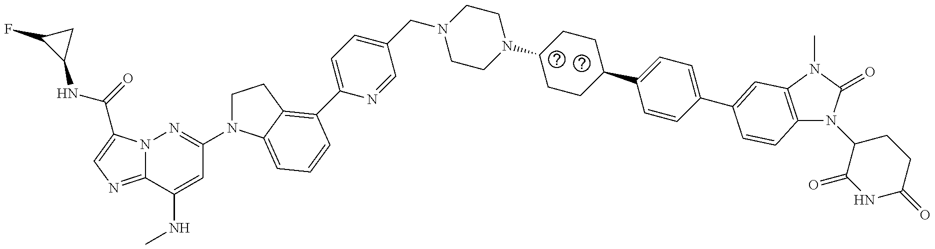 |
| K567 | 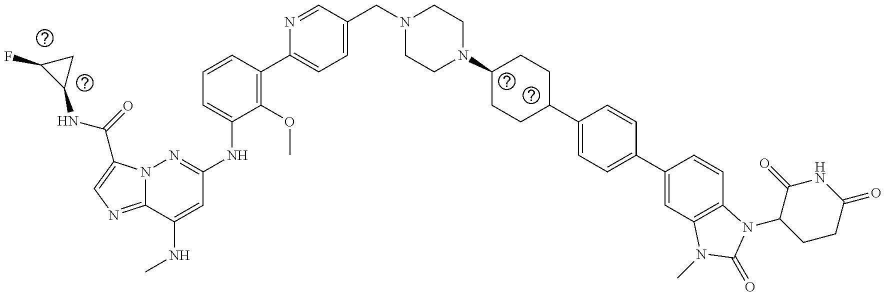 |
| K568 | 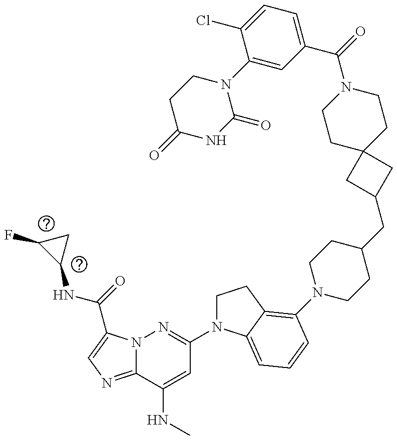 [SMILES: CNc1cc(N2CCc3c(N4CCC(CC5CC6(CCN(C(=O)c7ccc(Cl)c(N8CCC(=O)NC8=O)c7)CC6)C5)CC4)cccc32)nn2c(C(=O)N[C@@H]3C[C@@H]3F)cnc12] |
| K569 | 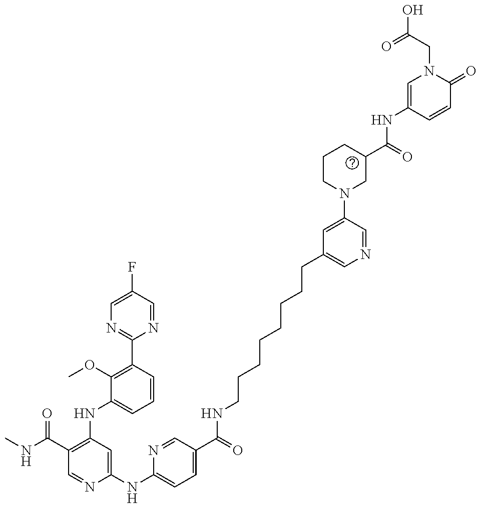 [SMILES: CNC(=O)c1cnc(Nc2ccc(C(=O)NCCCCCCCCc3cncc(N4CCCC(C(=O)Nc5ccc(=O)n(CC(=O)O)c5)C4)c3)cn2)cc1Nc1cccc(-c2ncc(F)cn2)c1OC] |
| K570 | 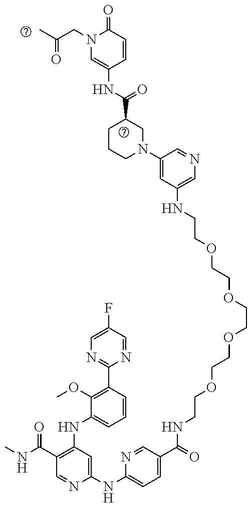 [SMILES: CNC(=O)c1cnc(Nc2ccc(C(=O)NCCOCCOCCOCCOCCNc3cncc(N4CCC[C@@H](C(=O)Nc5ccc(=O)n(CC(C)=O)c5)C4)c3)cn2)cc1Nc1cccc(-c2ncc(F)cn2)c1OC] |
| K571 | 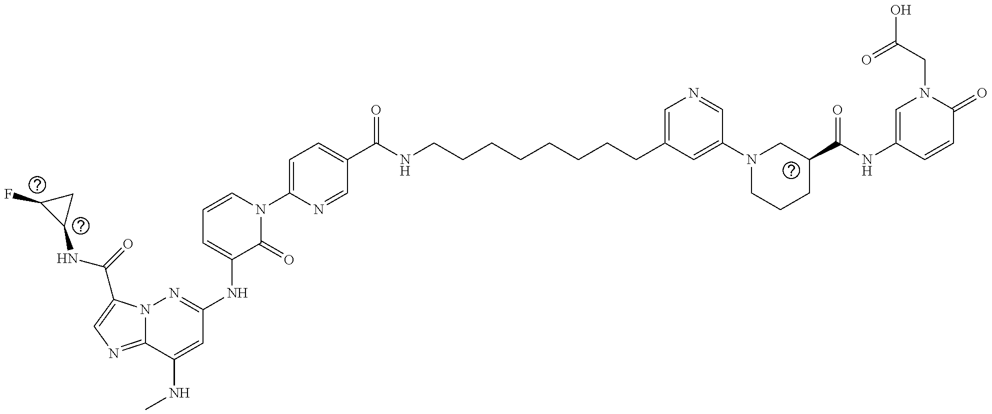 [SMILES: CNc1cc(Nc2cccn(-c3ccc(C(=O)NCCCCCCCCc4cncc(N5CCC[C@H](C(=O)Nc6ccc(=O)n(CC(=O)O)c6)C5)c4)cn3)c2=O)nn2c(C(=O)N[C@@H]3C[C@@H]3F)cnc12] |
| K572 | 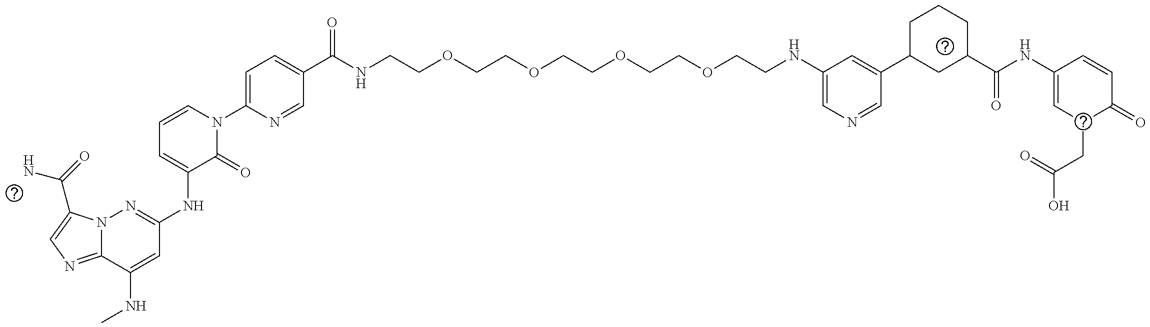 |
| K573 | 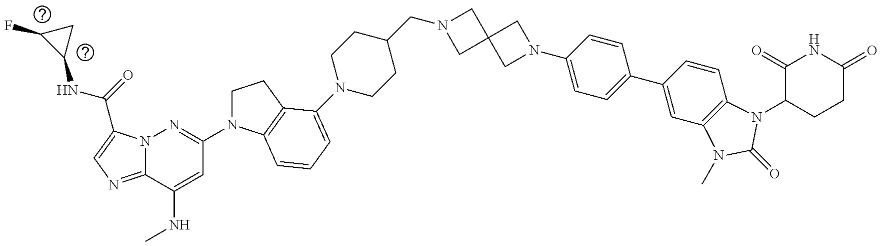 [SMILES: CNc1cc(N2CCc3c(N4CCC(CN5CC6(C5)CN(c5ccc(-c7ccc8c(c7)n(C)c(=O)n8C7CCC(=O)NC7=O)cc5)C6)CC4)cccc32)nn2c(C(=O)N[C@@H]3C[C@@H]3F)cnc12] |
| K574 | 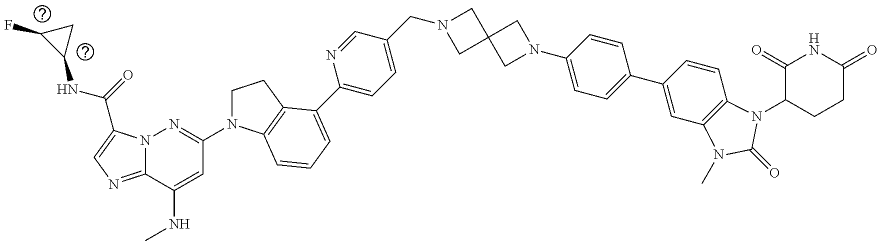 [SMILES: CNc1cc(N2CCc3c(-c4ccc(CN5CC6(C5)CN(c5ccc(-c7ccc8c(c7)n(C)c(=O)n8C7CCC(=O)NC7=O)cc5)C6)cn4)cccc32)nn2c(C(=O)N[C@@H]3C[C@@H]3F)cnc12] |
| K575 | 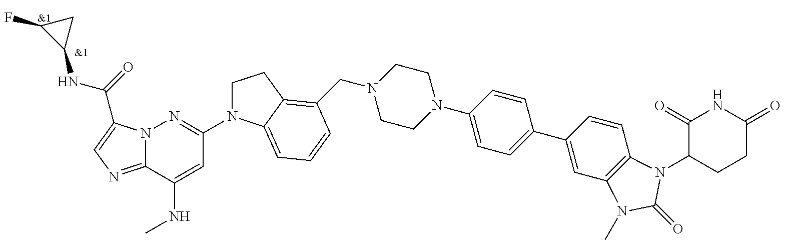 [SMILES: CNc1cc(N2CCc3c(CN4CCN(c5ccc(-c6ccc7c(c6)n(C)c(=O)n7C6CCC(=O)NC6=O)cc5)CC4)cccc32)nn2c(C(=O)N[C@@H]3C[C@@H]3F)cnc12] |
| K576 | 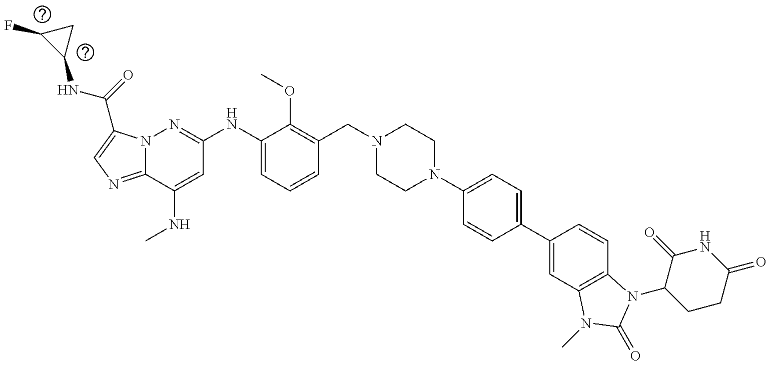 [SMILES: CNc1cc(Nc2cccc(CN3CCN(c4ccc(-c5ccc6c(c5)n(C)c(=O)n6C5CCC(=O)NC5=O)cc4)CC3)c2OC)nn2c(C(=O)N[C@@H]3C[C@@H]3F)cnc12] |
| K577 | 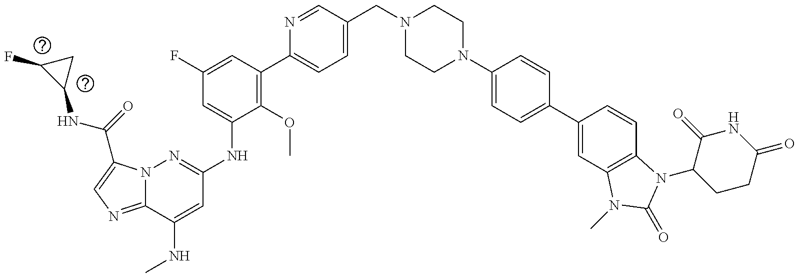 [SMILES: CNc1cc(Nc2cc(F)cc(-c3ccc(CN4CCN(c5ccc(-c6ccc7c(c6)n(C)c(=O)n7C6CCC(=O)NC6=O)cc5)CC4)cn3)c2OC)nn2c(C(=O)N[C@@H]3C[C@@H]3F)cnc12] |
| K578 | 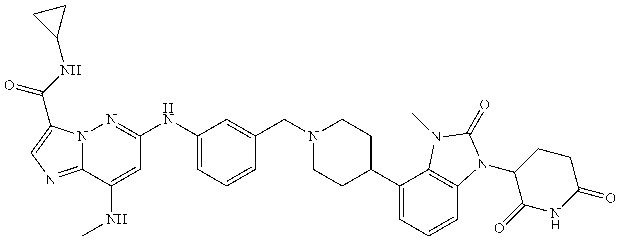 [SMILES: CNc1cc(Nc2cccc(CN3CCC(c4cccc5c4n(C)c(=O)n5C4CCC(=O)NC4=O)CC3)c2)nn2c(C(=O)NC3CC3)cnc12] |
| K579 | 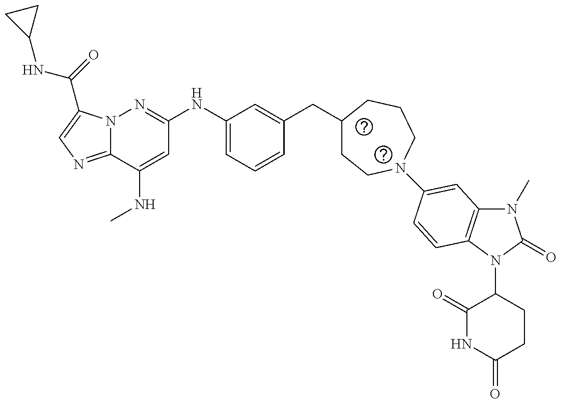 [SMILES: CNc1cc(Nc2cccc(CC3CCCN(c4ccc5c(c4)n(C)c(=O)n5C4CCC(=O)NC4=O)CC3)c2)nn2c(C(=O)NC3CC3)cnc12] |
| K580 | 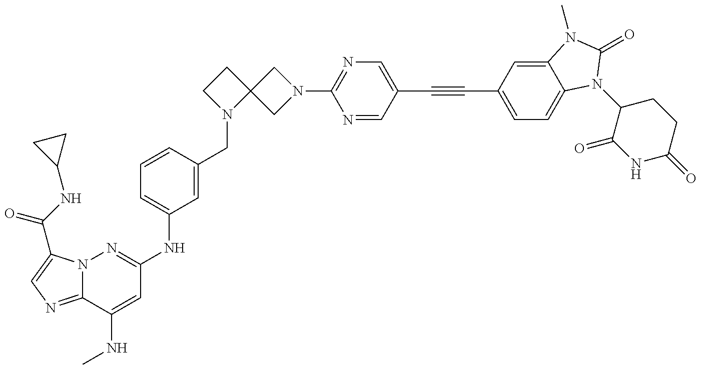 [SMILES: CNc1cc(Nc2cccc(CN3CCC34CN(c3ncc(C#Cc5ccc6c(c5)n(C)c(=O)n6C5CCC(=O)NC5=O)cn3)C4)c2)nn2c(C(=O)NC3CC3)cnc12] |
| K581 | 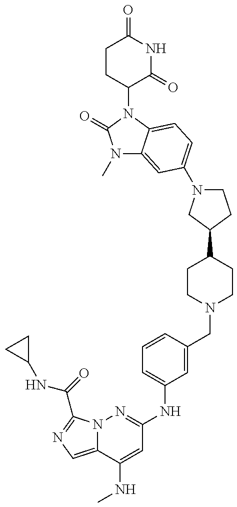 [SMILES: CNc1cc(Nc2cccc(CN3CCC([C@@H]4CCN(c5ccc6c(c5)n(C)c(=O)n6C5CCC(=O)NC5=O)C4)CC3)c2)nn2c(C(=O)NC3CC3)ncc12] |
| K582 | 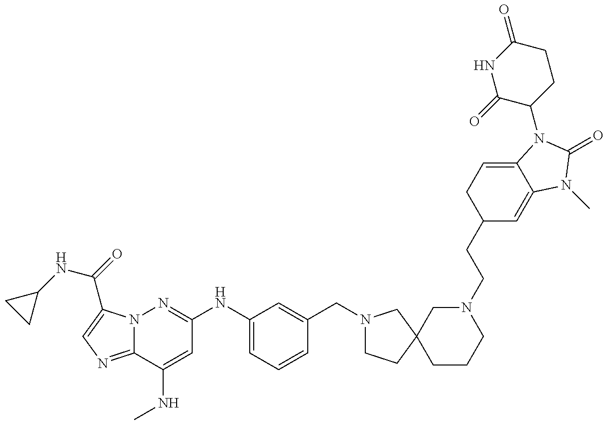 [SMILES: CNc1cc(Nc2cccc(CN3CCC4(CCCN(CCC5C=c6c(n(C7CCC(=O)NC7=O)c(=O)n6C)=CC5)C4)C3)c2)nn2c(C(=O)NC3CC3)cnc12] |
| K583 | 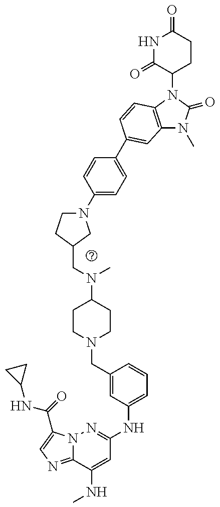 [SMILES: CNc1cc(Nc2cccc(CN3CCC(N(C)CC4CCN(c5ccc(-c6ccc7c(c6)n(C)c(=O)n7C6CCC(=O)NC6=O)cc5)C4)CC3)c2)nn2c(C(=O)NC3CC3)cnc12] |
| K584 | 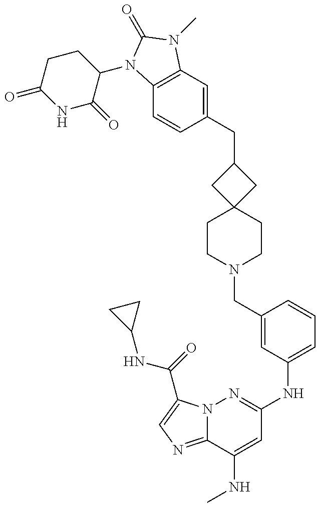 [SMILES: CNc1cc(Nc2cccc(CN3CCC4(CC3)CC(Cc3ccc5c(c3)n(C)c(=O)n5C3CCC(=O)NC3=O)C4)c2)nn2c(C(=O)NC3CC3)cnc12] |
| K585 | 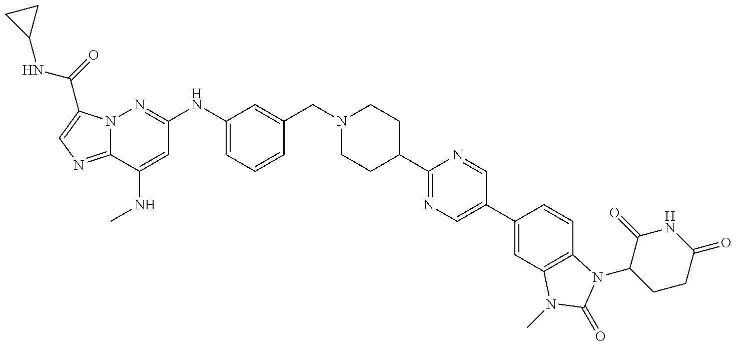 [SMILES: CNc1cc(Nc2cccc(CN3CCC(c4ncc(-c5ccc6c(c5)n(C)c(=O)n6C5CCC(=O)NC5=O)cn4)CC3)c2)nn2c(C(=O)NC3CC3)cnc12] |
| K586 | 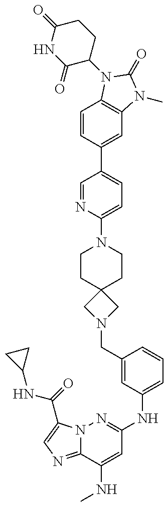 [SMILES: CNc1cc(Nc2cccc(CN3CC4(CCN(c5ccc(-c6ccc7c(c6)n(C)c(=O)n7C6CCC(=O)NC6=O)cn5)CC4)C3)c2)nn2c(C(=O)NC3CC3)cnc12] |
| K587 | 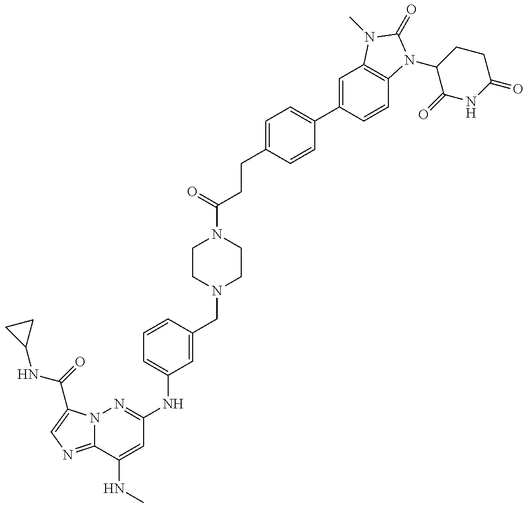 [SMILES: CNc1cc(Nc2cccc(CN3CCN(C(=O)CCc4ccc(-c5ccc6c(c5)n(C)c(=O)n6C5CCC(=O)NC5=O)cc4)CC3)c2)nn2c(C(=O)NC3CC3)cnc12] |
| K588 | 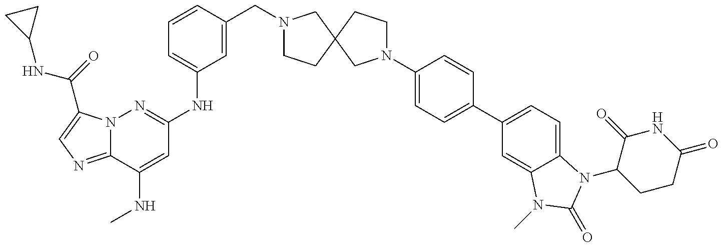 [SMILES: CNc1cc(Nc2cccc(CN3CCC4(CCN(c5ccc(-c6ccc7c(c6)n(C)c(=O)n7C6CCC(=O)NC6=O)cc5)C4)C3)c2)nn2c(C(=O)NC3CC3)cnc12] |
| K589 | 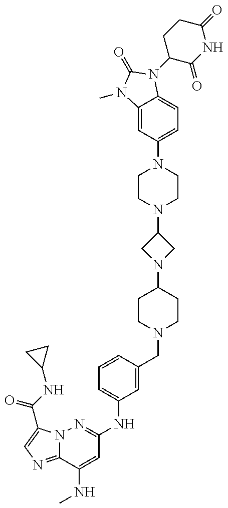 [SMILES: CNc1cc(Nc2cccc(CN3CCC(N4CC(N5CCN(c6ccc7c(c6)n(C)c(=O)n7C6CCC(=O)NC6=O)CC5)C4)CC3)c2)nn2c(C(=O)NC3CC3)cnc12] |
| K590 | 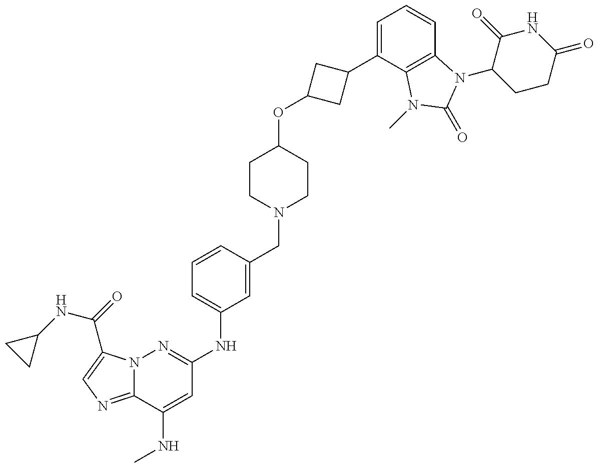 [SMILES: CNc1cc(Nc2cccc(CN3CCC(OC4CC(c5cccc6c5n(C)c(=O)n6C5CCC(=O)NC5=O)C4)CC3)c2)nn2c(C(=O)NC3CC3)cnc12] |
| K591 | 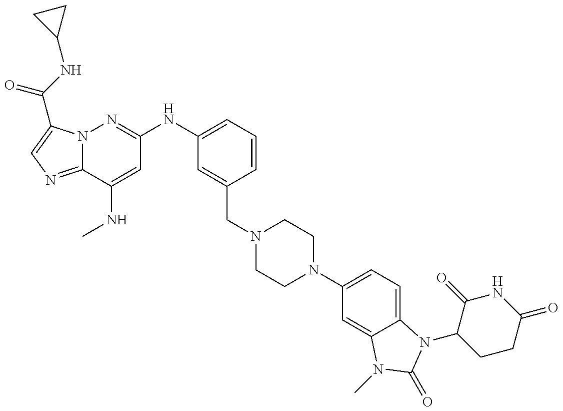 [SMILES: CNc1cc(Nc2cccc(CN3CCN(c4ccc5c(c4)n(C)c(=O)n5C4CCC(=O)NC4=O)CC3)c2)nn2c(C(=O)NC3CC3)cnc12] |
| K592 | 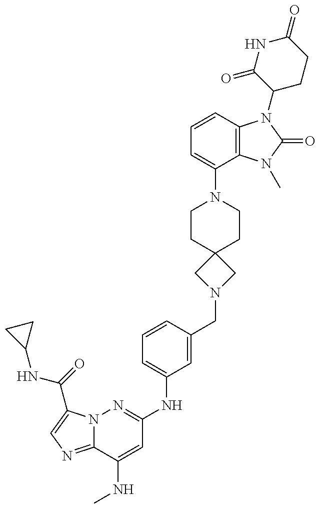 [SMILES: CNc1cc(Nc2cccc(CN3CC4(CCN(c5cccc6c5n(C)c(=O)n6C5CCC(=O)NC5=O)CC4)C3)c2)nn2c(C(=O)NC3CC3)cnc12] |
| K593 | 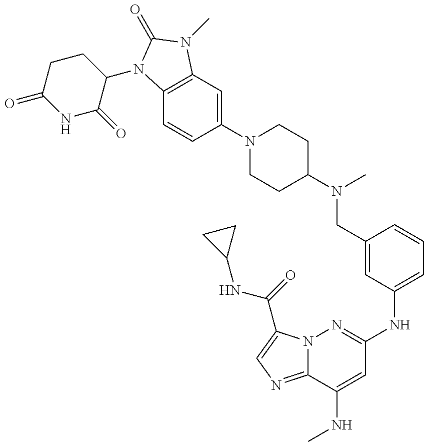 [SMILES: CNc1cc(Nc2cccc(CN(C)C3CCN(c4ccc5c(c4)n(C)c(=O)n5C4CCC(=O)NC4=O)CC3)c2)nn2c(C(=O)NC3CC3)cnc12] |
| K594 | 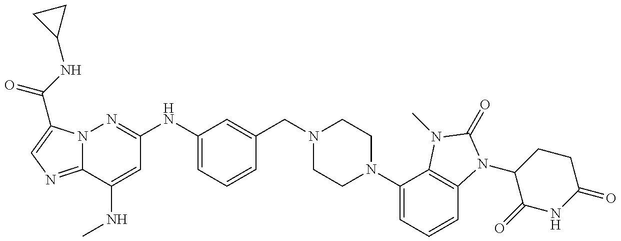 [SMILES: CNc1cc(Nc2cccc(CN3CCN(c4cccc5c4n(C)c(=O)n5C4CCC(=O)NC4=O)CC3)c2)nn2c(C(=O)NC3CC3)cnc12] |
| K595 | 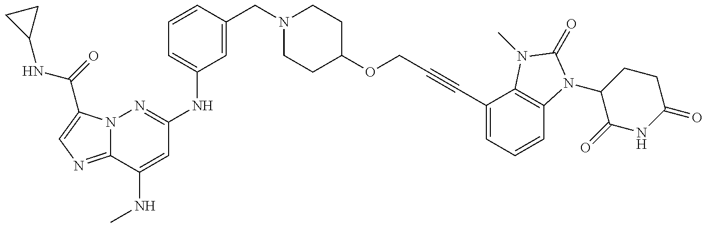 [SMILES: CNc1cc(Nc2cccc(CN3CCC(OCC#Cc4cccc5c4n(C)c(=O)n5C4CCC(=O)NC4=O)CC3)c2)nn2c(C(=O)NC3CC3)cnc12] |
| K596 | 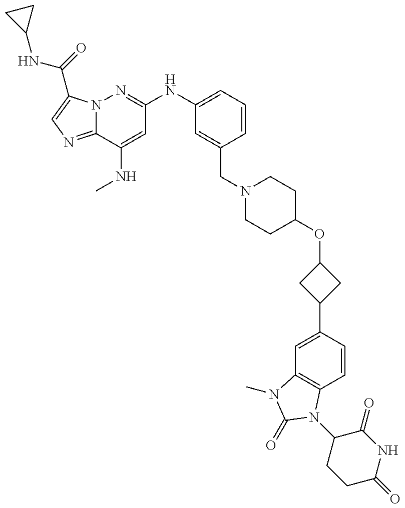 [SMILES: CNc1cc(Nc2cccc(CN3CCC(OC4CC(c5ccc6c(c5)n(C)c(=O)n6C5CCC(=O)NC5=O)C4)CC3)c2)nn2c(C(=O)NC3CC3)cnc12] |
| K597 | 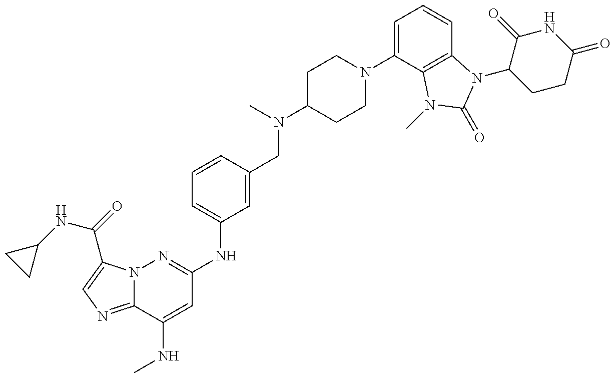 [SMILES: CNc1cc(Nc2cccc(CN(C)C3CCN(c4cccc5c4n(C)c(=O)n5C4CCC(=O)NC4=O)CC3)c2)nn2c(C(=O)NC3CC3)cnc12] |
| K598 | 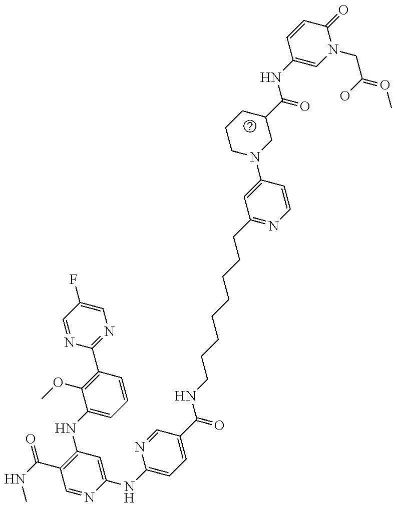 |
| K599 | 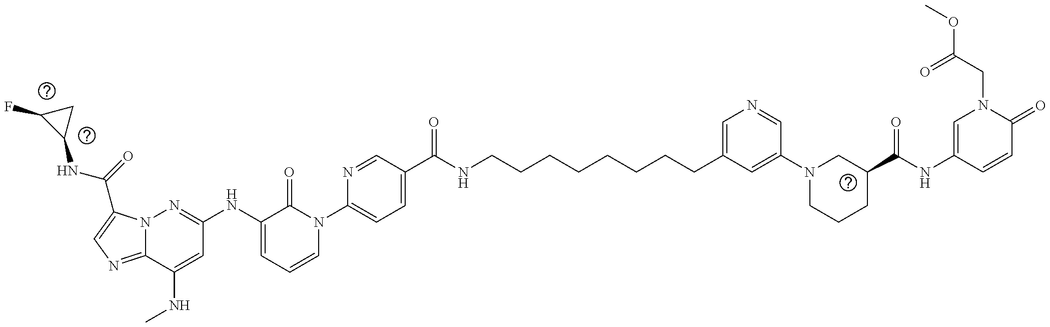 [SMILES: CNc1cc(Nc2cccn(-c3ccc(C(=O)NCCCCCCCCc4cncc(N5CCC[C@H](C(=O)Nc6ccc(=O)n(CC(=O)OC)c6)C5)c4)cn3)c2=O)nn2c(C(=O)N[C@@H]3C[C@@H]3F)cnc12] |
| K600 | 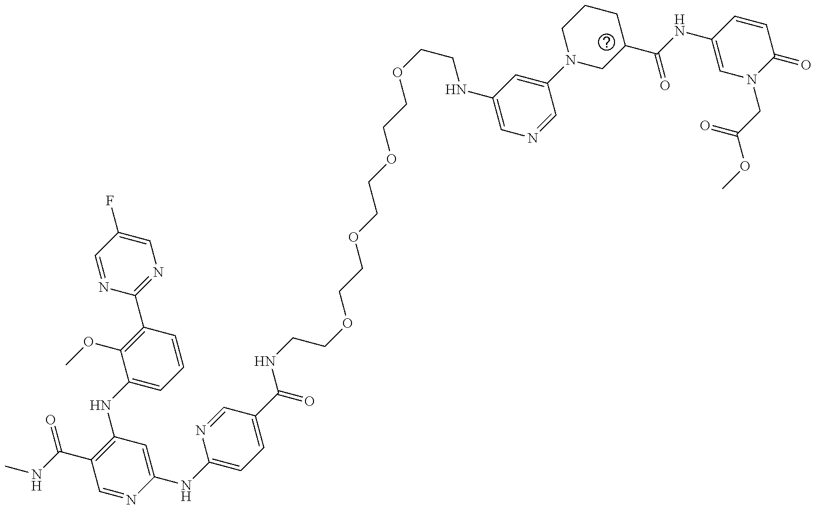 [SMILES: CNC(=O)c1cnc(Nc2ccc(C(=O)NCCOCCOCCOCCOCCNc3cncc(N4CCCC(C(=O)Nc5ccc(=O)n(CC(=O)OC)c5)C4)c3)cn2)cc1Nc1cccc(-c2ncc(F)cn2)c1OC] |
| K601 | 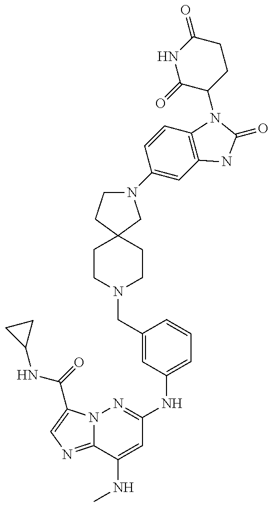 [SMILES: CNc1cc(Nc2cccc(CN3CCC4(CC3)CCN(c3ccc5c(c3)[nH]c(=O)n5C3CCC(=O)NC3=O)C4)c2)nn2c(C(=O)NC3CC3)cnc12] |
| K602 | 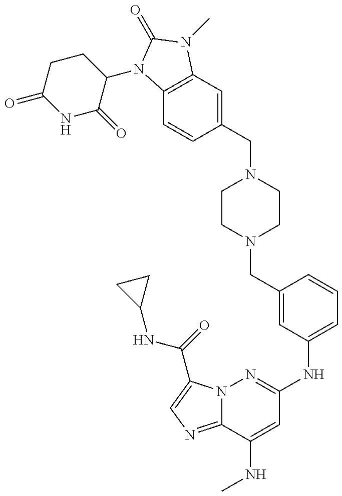 [SMILES: CNc1cc(Nc2cccc(CN3CCN(Cc4ccc5c(c4)n(C)c(=O)n5C4CCC(=O)NC4=O)CC3)c2)nn2c(C(=O)NC3CC3)cnc12] |
| K603 | 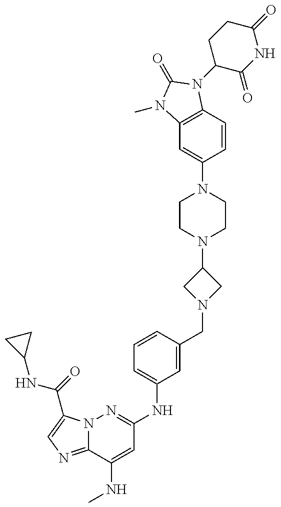 [SMILES: CNc1cc(Nc2cccc(CN3CC(N4CCN(c5ccc6c(c5)n(C)c(=O)n6C5CCC(=O)NC5=O)CC4)C3)c2)nn2c(C(=O)NC3CC3)cnc12] |
| K604 | 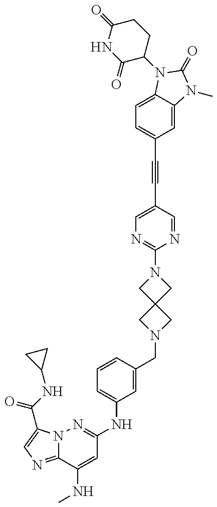 [SMILES: CNc1cc(Nc2cccc(CN3CC4(C3)CN(c3ncc(C#Cc5ccc6c(c5)n(C)c(=O)n6C5CCC(=O)NC5=O)cn3)C4)c2)nn2c(C(=O)NC3CC3)cnc12] |
| K605 | 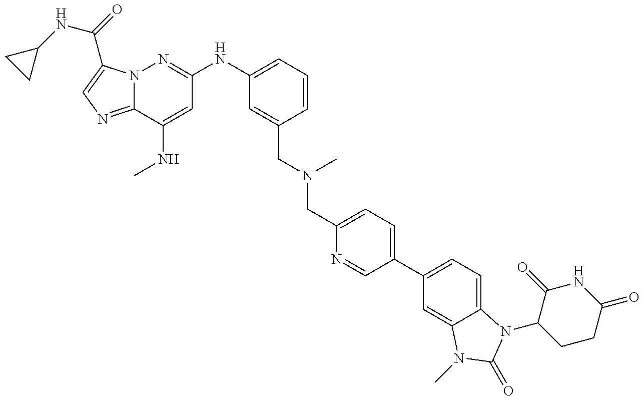 [SMILES: CNc1cc(Nc2cccc(CN(C)Cc3ccc(-c4ccc5c(c4)n(C)c(=O)n5C4CCC(=O)NC4=O)cn3)c2)nn2c(C(=O)NC3CC3)cnc12] |
| K606 | 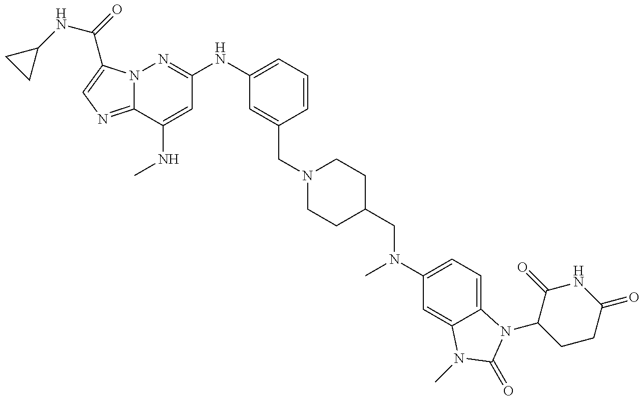 [SMILES: CNc1cc(Nc2cccc(CN3CCC(CN(C)c4ccc5c(c4)n(C)c(=O)n5C4CCC(=O)NC4=O)CC3)c2)nn2c(C(=O)NC3CC3)cnc12] |
| K607 | 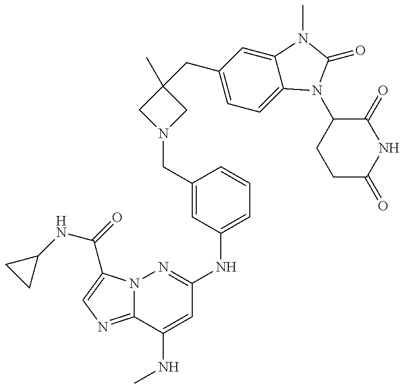 [SMILES: CNc1cc(Nc2cccc(CN3CC(C)(Cc4ccc5c(c4)n(C)c(=O)n5C4CCC(=O)NC4=O)C3)c2)nn2c(C(=O)NC3CC3)cnc12] |
| K608 | 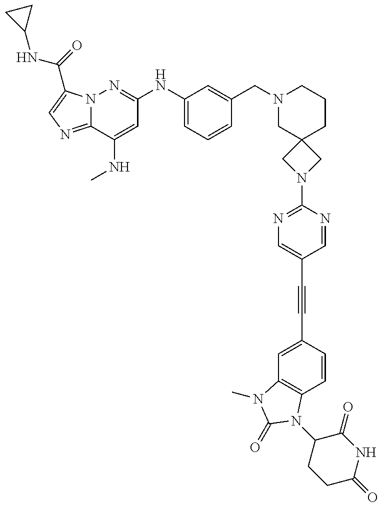 [SMILES: CNc1cc(Nc2cccc(CN3CCCC4(C3)CN(c3ncc(C#Cc5ccc6c(c5)n(C)c(=O)n6C5CCC(=O)NC5=O)cn3)C4)c2)nn2c(C(=O)NC3CC3)cnc12] |
| K609 | 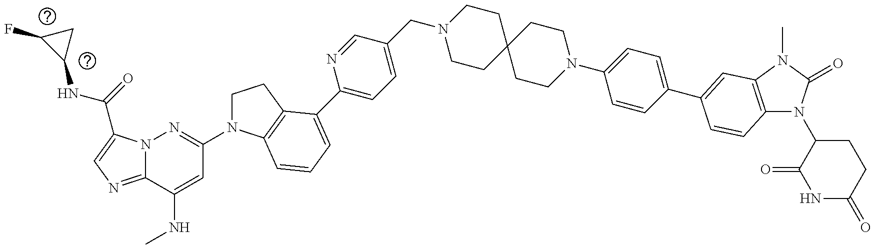 [SMILES: CNc1cc(N2CCc3c(-c4ccc(CN5CCC6(CC5)CCN(c5ccc(-c7ccc8c(c7)n(C)c(=O)n8C7CCC(=O)NC7=O)cc5)CC6)cn4)cccc32)nn2c(C(=O)N[C@@H]3C[C@@H]3F)cnc12] |
| K610 | 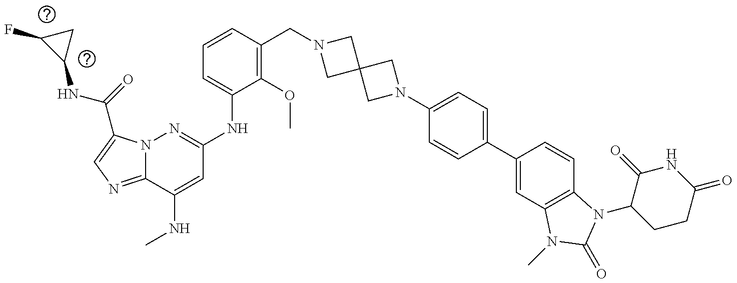 [SMILES: CNc1cc(Nc2cccc(CN3CC4(C3)CN(c3ccc(-c5ccc6c(c5)n(C)c(=O)n6C5CCC(=O)NC5=O)cc3)C4)c2OC)nn2c(C(=O)N[C@@H]3C[C@@H]3F)cnc12] |
| K611 | 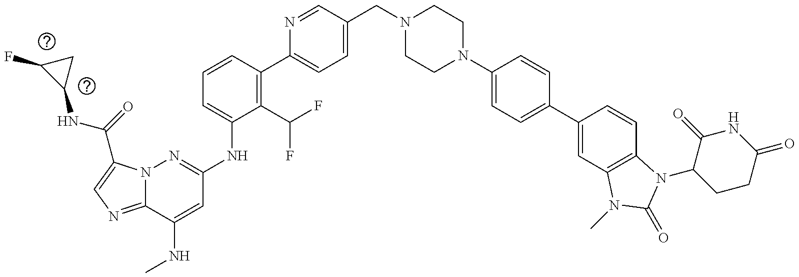 [SMILES: CNc1cc(Nc2cccc(-c3ccc(CN4CCN(c5ccc(-c6ccc7c(c6)n(C)c(=O)n7C6CCC(=O)NC6=O)cc5)CC4)cn3)c2C(F)F)nn2c(C(=O)N[C@@H]3C[C@@H]3F)cnc12] |
| K612 | 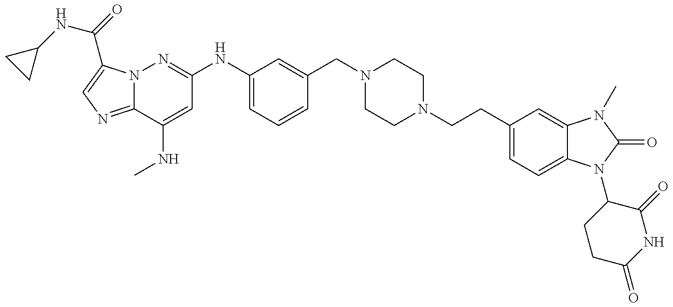 [SMILES: CNc1cc(Nc2cccc(CN3CCN(CCc4ccc5c(c4)n(C)c(=O)n5C4CCC(=O)NC4=O)CC3)c2)nn2c(C(=O)NC3CC3)cnc12] |
| K613 | 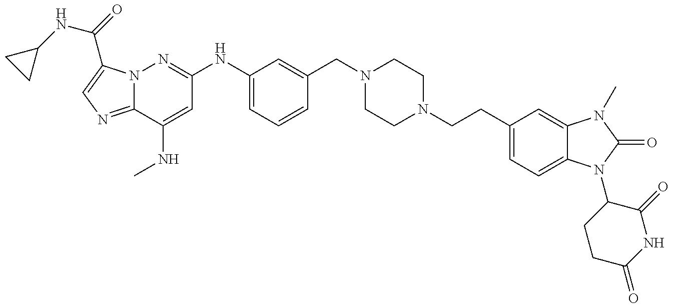 [SMILES: CNc1cc(Nc2cccc(CN3CCN(CCc4ccc5c(c4)n(C)c(=O)n5C4CCC(=O)NC4=O)CC3)c2)nn2c(C(=O)NC3CC3)cnc12] |
| K614 | 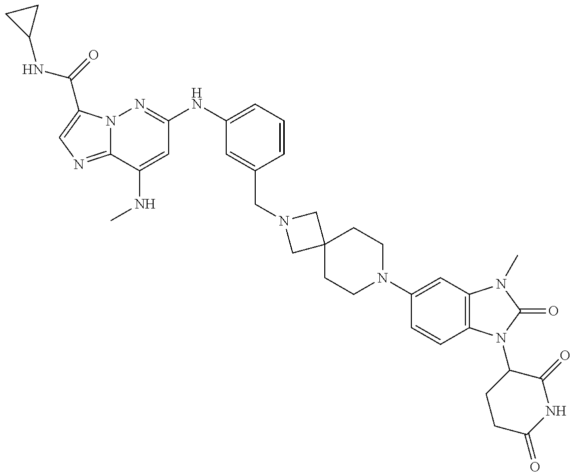 [SMILES: CNc1cc(Nc2cccc(CN3CC4(CCN(c5ccc6c(c5)n(C)c(=O)n6C5CCC(=O)NC5=O)CC4)C3)c2)nn2c(C(=O)NC3CC3)cnc12] |
| K615 | 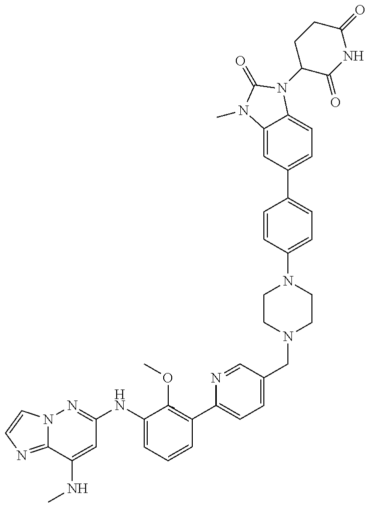 |
| K616 | 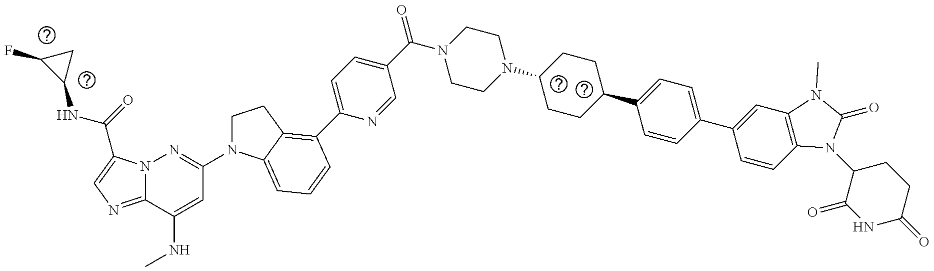 [SMILES: CNc1cc(N2CCc3c(-c4ccc(C(=O)N5CCN([C@H]6CC[C@H](c7ccc(-c8ccc9c(c8)n(C)c(=O)n9C8CCC(=O)NC8=O)cc7)CC6)CC5)cn4)cccc32)nn2c(C(=O)N[C@@H]3C[C@@H]3F)cnc12] |
| K617 | 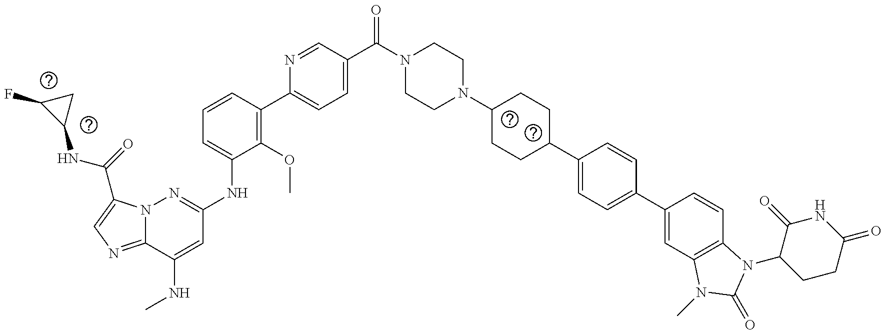 [SMILES: CNc1cc(Nc2cccc(-c3ccc(C(=O)N4CCN(C5CCC(c6ccc(-c7ccc8c(c7)n(C)c(=O)n8C7CCC(=O)NC7=O)cc6)CC5)CC4)cn3)c2OC)nn2c(C(=O)N[C@@H]3C[C@@H]3F)cnc12] |
| K618 | 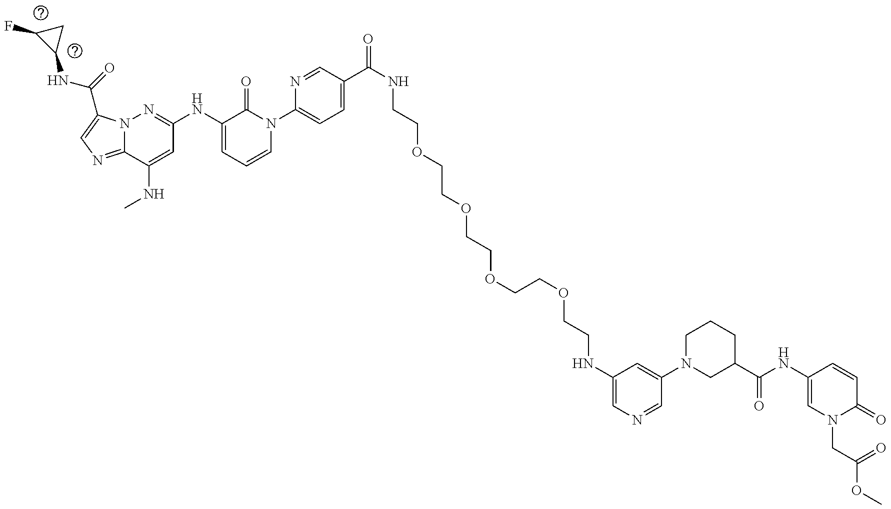 [SMILES: CNc1cc(Nc2cccn(-c3ccc(C(=O)NCCOCCOCCOCCOCCNc4cncc(N5CCCC(C(=O)Nc6ccc(=O)n(CC(=O)OC)c6)C5)c4)cn3)c2=O)nn2c(C(=O)N[C@@H]3C[C@@H]3F)cnc12] |
| K619 | 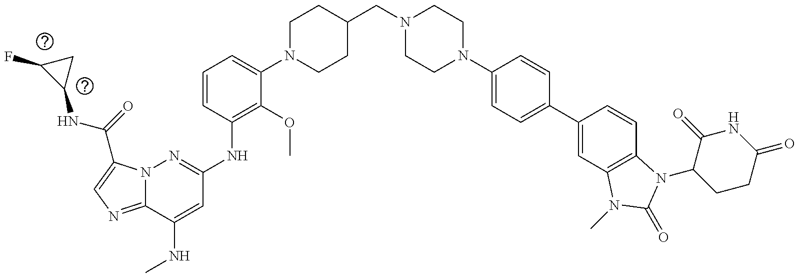 [SMILES: CNc1cc(Nc2cccc(N3CCC(CN4CCN(c5ccc(-c6ccc7c(c6)n(C)c(=O)n7C6CCC(=O)NC6=O)cc5)CC4)CC3)c2OC)nn2c(C(=O)N[C@@H]3C[C@@H]3F)cnc12] |
| K620 | 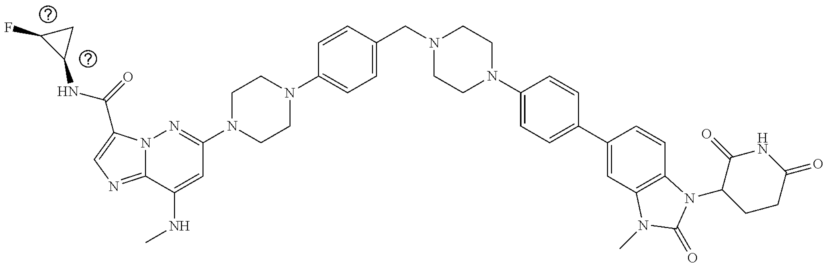 [SMILES: CNc1cc(N2CCN(c3ccc(CN4CCN(c5ccc(-c6ccc7c(c6)n(C)c(=O)n7C6CCC(=O)NC6=O)cc5)CC4)cc3)CC2)nn2c(C(=O)N[C@@H]3C[C@@H]3F)cnc12] |
| K621 | 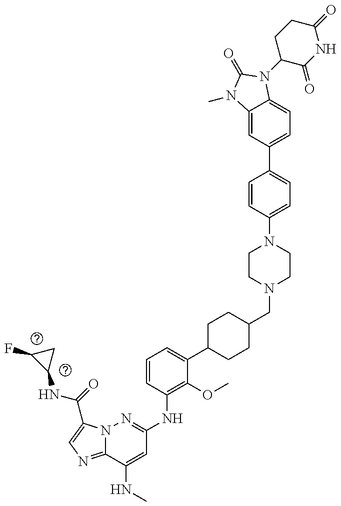 [SMILES: CNc1cc(Nc2cccc(C3CCC(CN4CCN(c5ccc(-c6ccc7c(c6)n(C)c(=O)n7C6CCC(=O)NC6=O)cc5)CC4)CC3)c2OC)nn2c(C(=O)N[C@@H]3C[C@@H]3F)cnc12] |
| K622 | 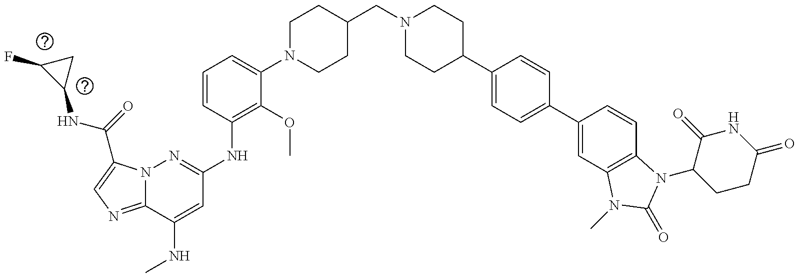 [SMILES: CNc1cc(Nc2cccc(N3CCC(CN4CCC(c5ccc(-c6ccc7c(c6)n(C)c(=O)n7C6CCC(=O)NC6=O)cc5)CC4)CC3)c2OC)nn2c(C(=O)N[C@@H]3C[C@@H]3F)cnc12] |
| K623 | 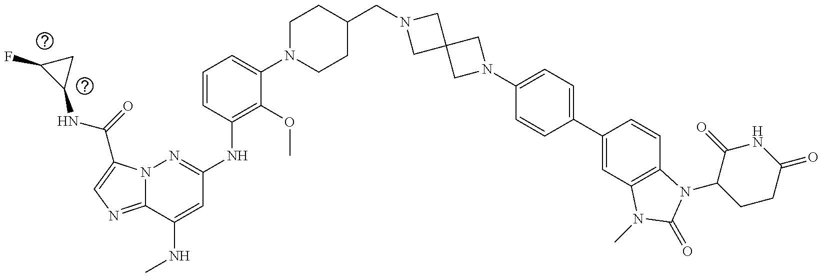 [SMILES: CNc1cc(Nc2cccc(N3CCC(CN4CC5(C4)CN(c4ccc(-c6ccc7c(c6)n(C)c(=O)n7C6CCC(=O)NC6=O)cc4)C5)CC3)c2OC)nn2c(C(=O)N[C@@H]3C[C@@H]3F)cnc12] |
| K624 | 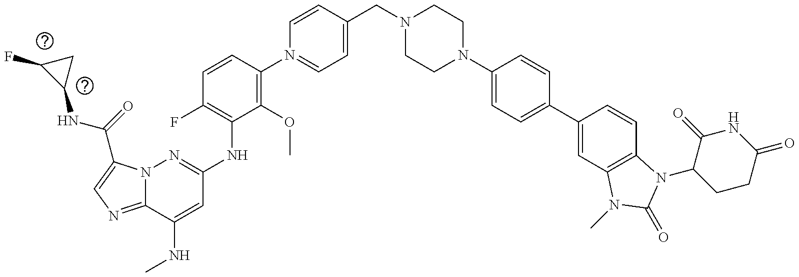 |
| K625 | 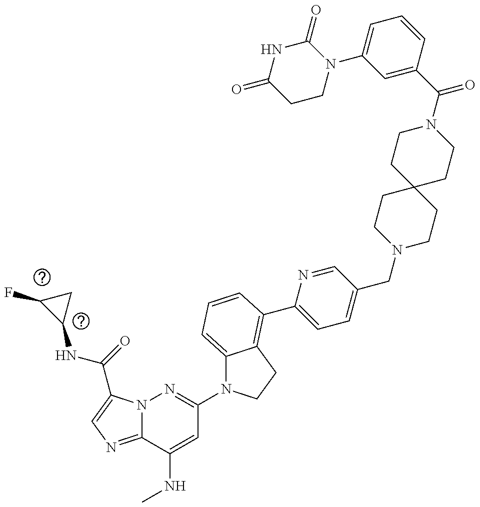 [SMILES: CNc1cc(N2CCc3c(-c4ccc(CN5CCC6(CC5)CCN(C(=O)c5cccc(N7CCC(=O)NC7=O)c5)CC6)cn4)cccc32)nn2c(C(=O)N[C@@H]3C[C@@H]3F)cnc12] |
| K626 | 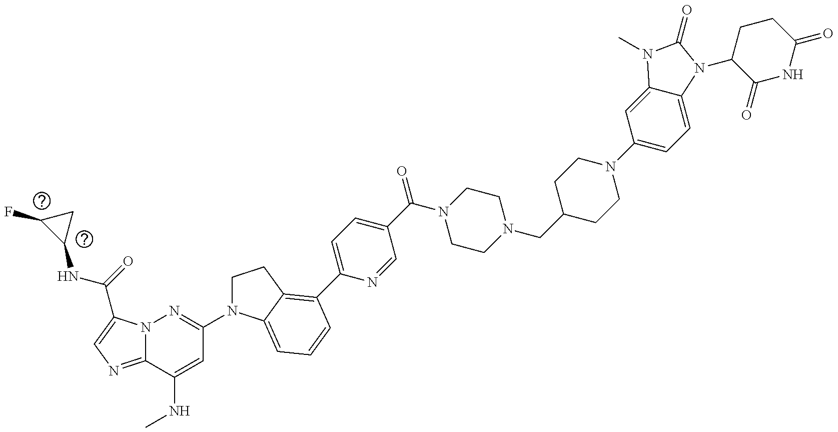 [SMILES: CNc1cc(N2CCc3c(-c4ccc(C(=O)N5CCN(CC6CCN(c7ccc8c(c7)n(C)c(=O)n8C7CCC(=O)NC7=O)CC6)CC5)cn4)cccc32)nn2c(C(=O)N[C@@H]3C[C@@H]3F)cnc12] |
| K627 | 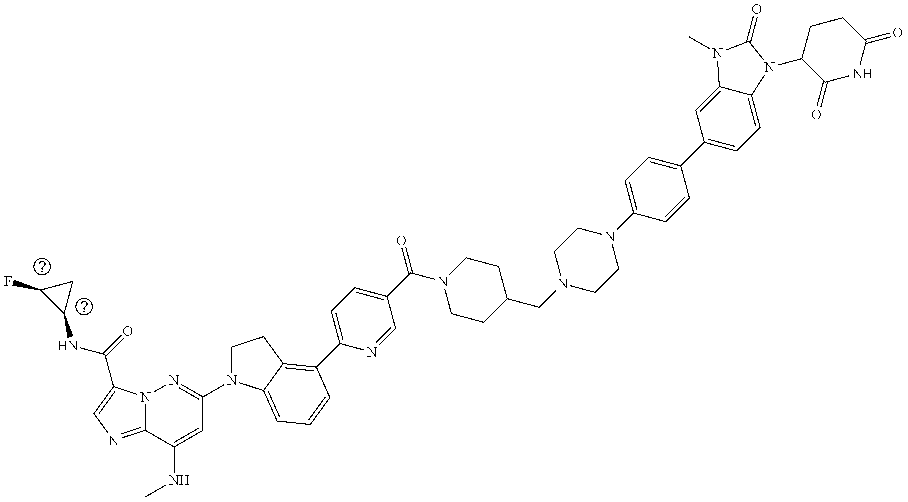 [SMILES: CNc1cc(N2CCc3c(-c4ccc(C(=O)N5CCC(CN6CCN(c7ccc(-c8ccc9c(c8)n(C)c(=O)n9C8CCC(=O)NC8=O)cc7)CC6)CC5)cn4)cccc32)nn2c(C(=O)N[C@@H]3C[C@@H]3F)cnc12] |
| K628 | 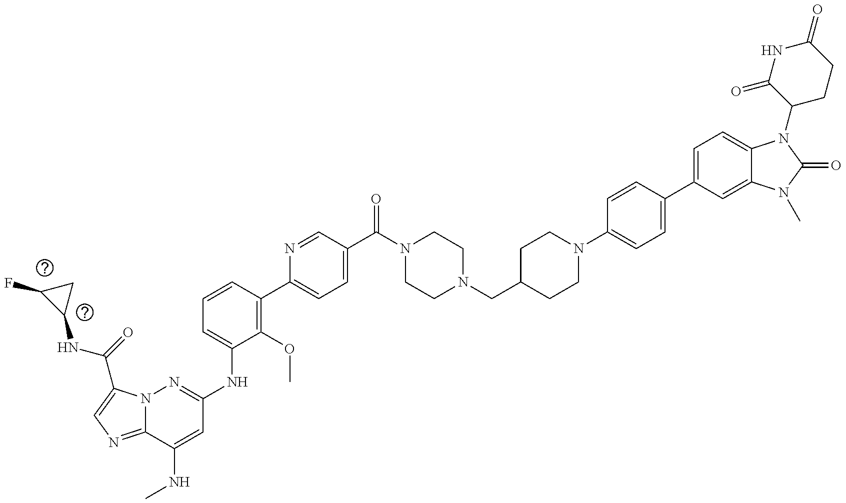 [SMILES: CNc1cc(Nc2cccc(-c3ccc(C(=O)N4CCN(CC5CCN(c6ccc(-c7ccc8c(c7)n(C)c(=O)n8C7CCC(=O)NC7=O)cc6)CC5)CC4)cn3)c2OC)nn2c(C(=O)N[C@@H]3C[C@@H]3F)cnc12] |
| K629 | 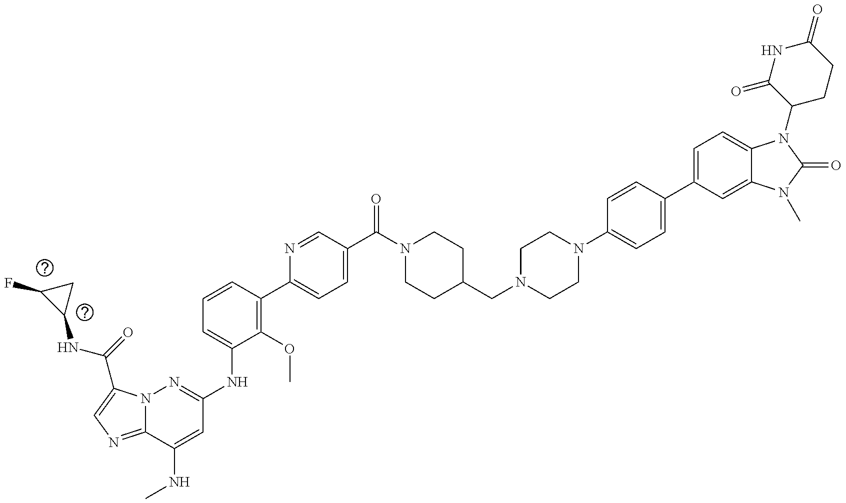 [SMILES: CNc1cc(Nc2cccc(-c3ccc(C(=O)N4CCC(CN5CCN(c6ccc(-c7ccc8c(c7)n(C)c(=O)n8C7CCC(=O)NC7=O)cc6)CC5)CC4)cn3)c2OC)nn2c(C(=O)N[C@@H]3C[C@@H]3F)cnc12] |
| K630 | 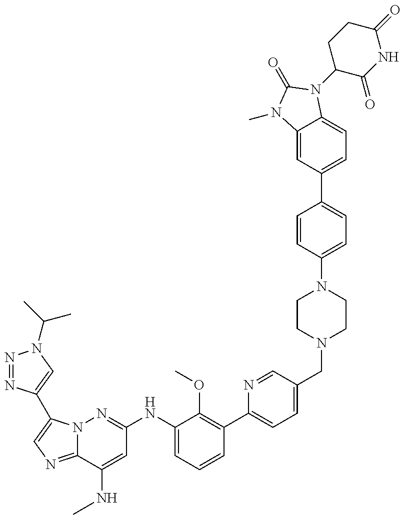 [SMILES: CNc1cc(Nc2cccc(-c3ccc(CN4CCN(c5ccc(-c6ccc7c(c6)n(C)c(=O)n7C6CCC(=O)NC6=O)cc5)CC4)cn3)c2OC)nn2c(-c3cn(C(C)C)nn3)cnc12] |
| K631 | 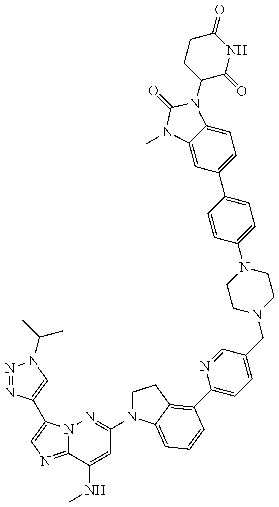 [SMILES: CNc1cc(N2CCc3c(-c4ccc(CN5CCN(c6ccc(-c7ccc8c(c7)n(C)c(=O)n8C7CCC(=O)NC7=O)cc6)CC5)cn4)cccc32)nn2c(-c3cn(C(C)C)nn3)cnc12] |
| K632 | 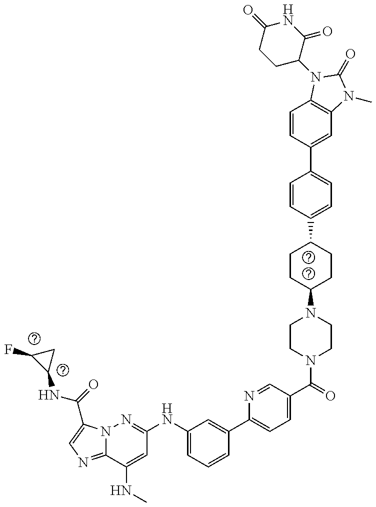 [SMILES: CNc1cc(Nc2cccc(-c3ccc(C(=O)N4CCN([C@H]5CC[C@H](c6ccc(-c7ccc8c(c7)n(C)c(=O)n8C7CCC(=O)NC7=O)cc6)CC5)CC4)cn3)c2)nn2c(C(=O)N[C@@H]3C[C@@H]3F)cnc12] |
| K633 | 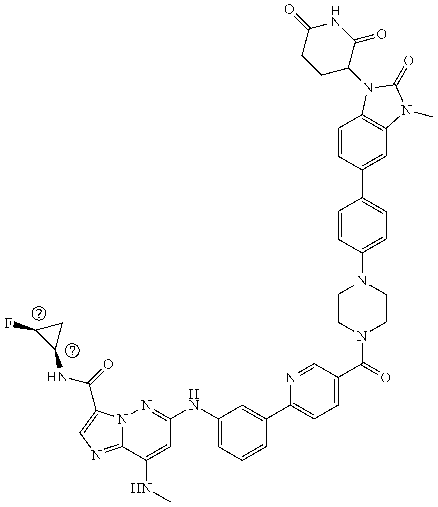 [SMILES: CNc1cc(Nc2cccc(-c3ccc(C(=O)N4CCN(c5ccc(-c6ccc7c(c6)n(C)c(=O)n7C6CCC(=O)NC6=O)cc5)CC4)cn3)c2)nn2c(C(=O)N[C@@H]3C[C@@H]3F)cnc12] |
| K634 | 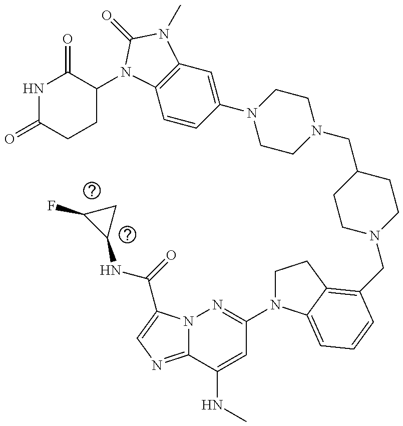 [SMILES: CNc1cc(N2CCc3c(CN4CCC(CN5CCN(c6ccc7c(c6)n(C)c(=O)n7C6CCC(=O)NC6=O)CC5)CC4)cccc32)nn2c(C(=O)N[C@@H]3C[C@@H]3F)cnc12] |
| K635 | 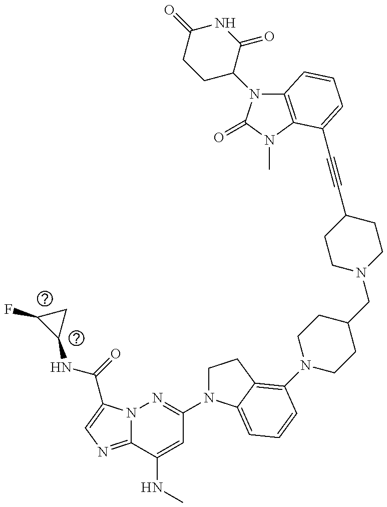 [SMILES: CNc1cc(N2CCc3c(N4CCC(CN5CCC(C#Cc6cccc7c6n(C)c(=O)n7C6CCC(=O)NC6=O)CC5)CC4)cccc32)nn2c(C(=O)N[C@@H]3C[C@@H]3F)cnc12] |
| K636 | 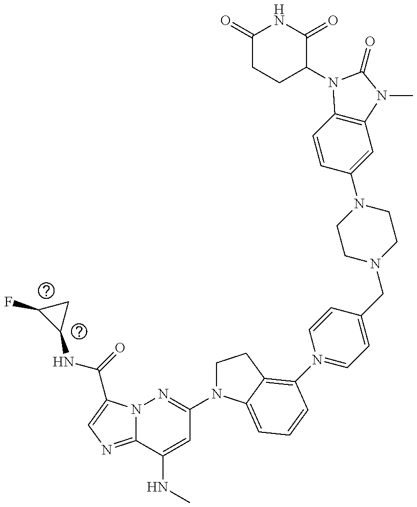 |
| K637 | 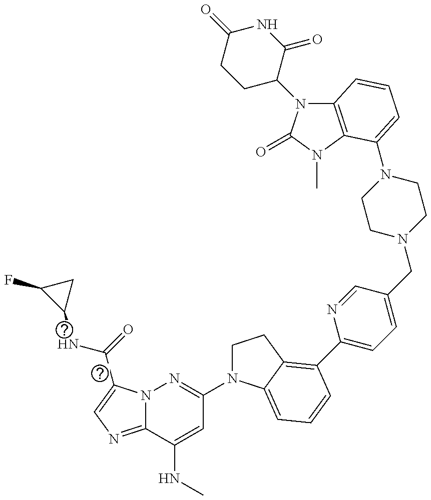 [SMILES: CNc1cc(N2CCc3c(-c4ccc(CN5CCN(c6cccc7c6n(C)c(=O)n7C6CCC(=O)NC6=O)CC5)cn4)cccc32)nn2c(C(=O)N[C@@H]3C[C@@H]3F)cnc12] |
| K638 | 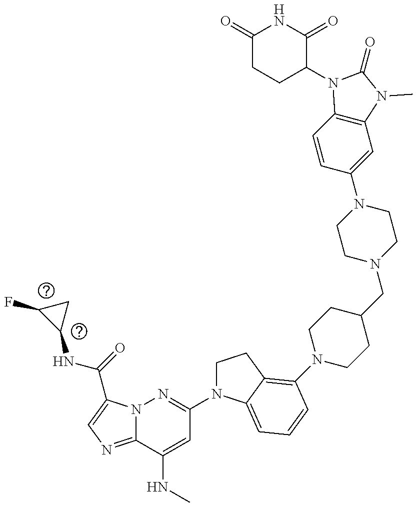 [SMILES: CNc1cc(N2CCc3c(N4CCC(CN5CCN(c6ccc7c(c6)n(C)c(=O)n7C6CCC(=O)NC6=O)CC5)CC4)cccc32)nn2c(C(=O)N[C@@H]3C[C@@H]3F)cnc12] |
| K639 | 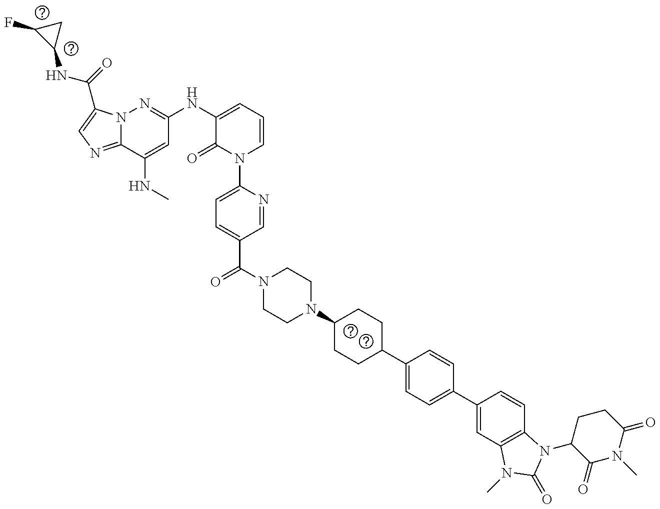 |
| K640 | 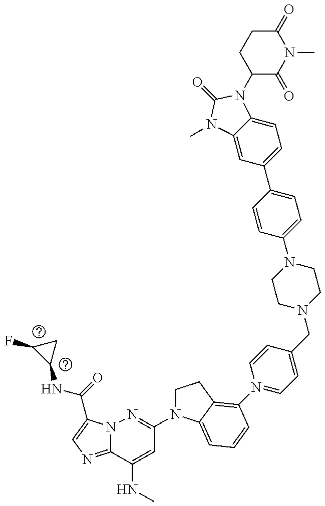 |
| K641 | 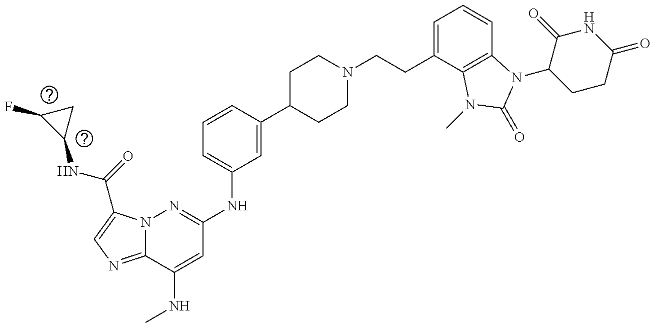 [SMILES: CNc1cc(Nc2cccc(C3CCN(CCc4cccc5c4n(C)c(=O)n5C4CCC(=O)NC4=O)CC3)c2)nn2c(C(=O)N[C@@H]3C[C@@H]3F)cnc12] |
| K642 | 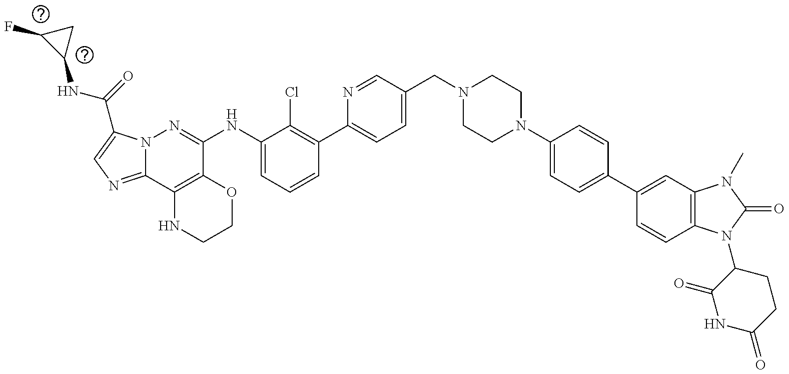 [SMILES: Cn1c(=O)n(C2CCC(=O)NC2=O)c2ccc(-c3ccc(N4CCN(Cc5ccc(-c6cccc(Nc7nn8c(C(=O)N[C@@H]9C[C@@H]9F)cnc8c8c7OCCN8)c6Cl)nc5)CC4)cc3)cc21] |
| K643 | 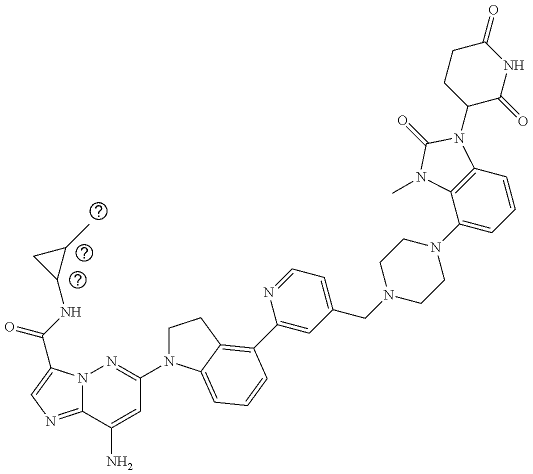 [SMILES: CC1CC1NC(=O)c1cnc2c(N)cc(N3CCc4c(-c5cc(CN6CCN(c7cccc8c7n(C)c(=O)n8C7CCC(=O)NC7=O)CC6)ccn5)cccc43)nn12] |
| K644 | 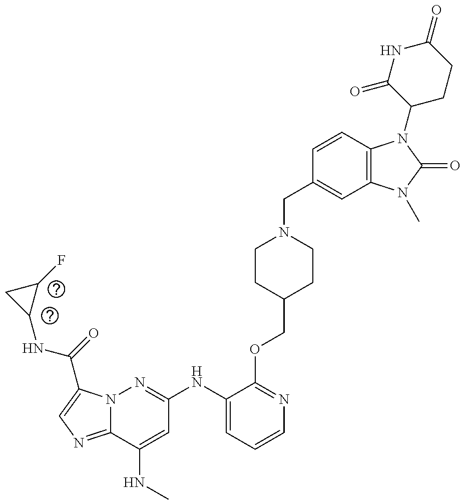 [SMILES: CNc1cc(Nc2cccnc2OCC2CCN(Cc3ccc4c(c3)n(C)c(=O)n4C3CCC(=O)NC3=O)CC2)nn2c(C(=O)NC3CC3F)cnc12] |
| K645 | 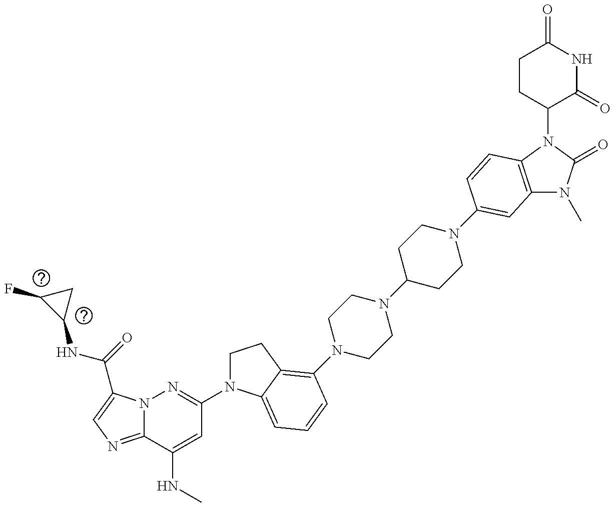 [SMILES: CNc1cc(N2CCc3c(N4CCN(C5CCN(c6ccc7c(c6)n(C)c(=O)n7C6CCC(=O)NC6=O)CC5)CC4)cccc32)nn2c(C(=O)N[C@@H]3C[C@@H]3F)cnc12] |
| K646 | 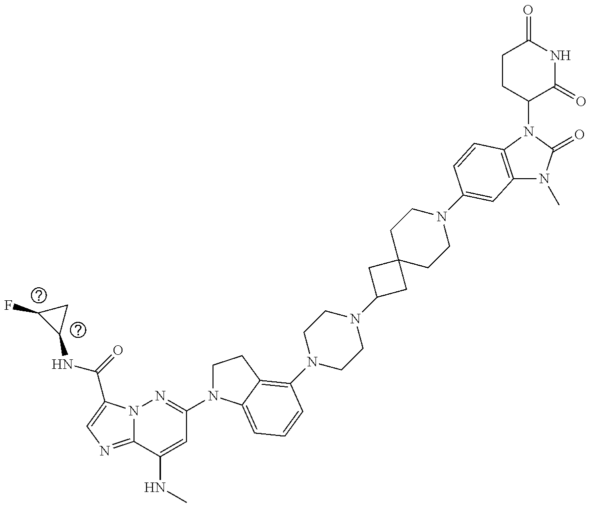 [SMILES: CNc1cc(N2CCc3c(N4CCN(C5CC6(CCN(c7ccc8c(c7)n(C)c(=O)n8C7CCC(=O)NC7=O)CC6)C5)CC4)cccc32)nn2c(C(=O)N[C@@H]3C[C@@H]3F)cnc12] |
| K647 | 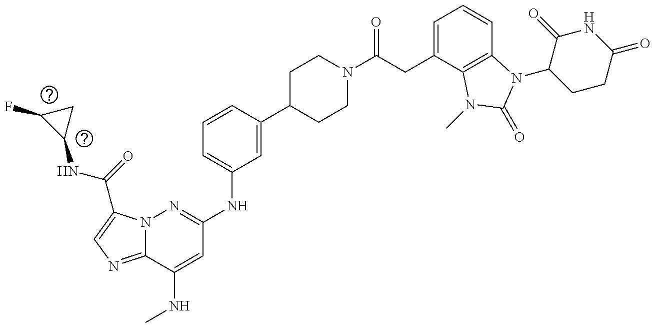 [SMILES: CNc1cc(Nc2cccc(C3CCN(C(=O)Cc4cccc5c4n(C)c(=O)n5C4CCC(=O)NC4=O)CC3)c2)nn2c(C(=O)N[C@@H]3C[C@@H]3F)cnc12] |
| K648 | 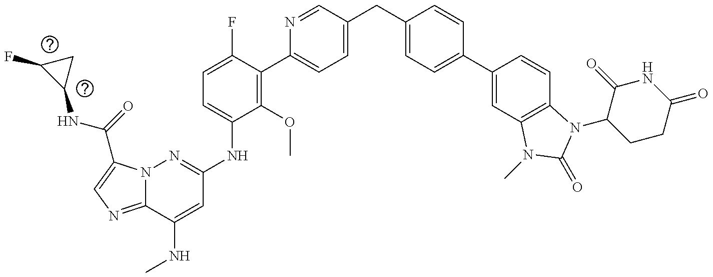 [SMILES: CNc1cc(Nc2ccc(F)c(-c3ccc(Cc4ccc(-c5ccc6c(c5)n(C)c(=O)n6C5CCC(=O)NC5=O)cc4)cn3)c2OC)nn2c(C(=O)N[C@@H]3C[C@@H]3F)cnc12] |
| K649 | 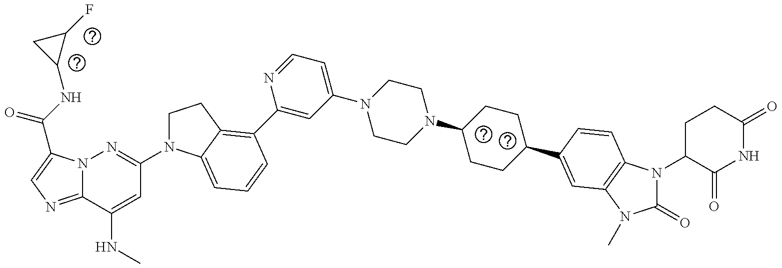 [SMILES: CNc1cc(N2CCc3c(-c4cc(N5CCN([C@H]6CC[C@@H](c7ccc8c(c7)n(C)c(=O)n8C7CCC(=O)NC7=O)CC6)CC5)ccn4)cccc32)nn2c(C(=O)NC3CC3F)cnc12] |
| K650 | 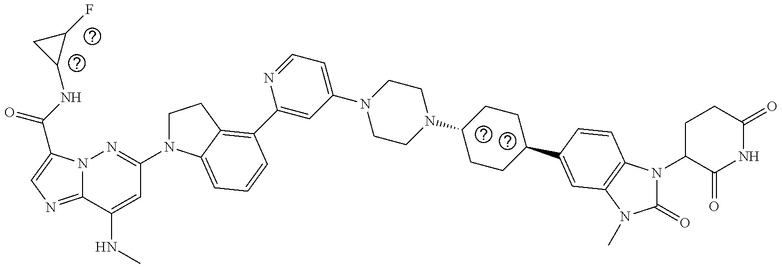 [SMILES: CNc1cc(N2CCc3c(-c4cc(N5CCN([C@H]6CC[C@H](c7ccc8c(c7)n(C)c(=O)n8C7CCC(=O)NC7=O)CC6)CC5)ccn4)cccc32)nn2c(C(=O)NC3CC3F)cnc12] |
| K651 | 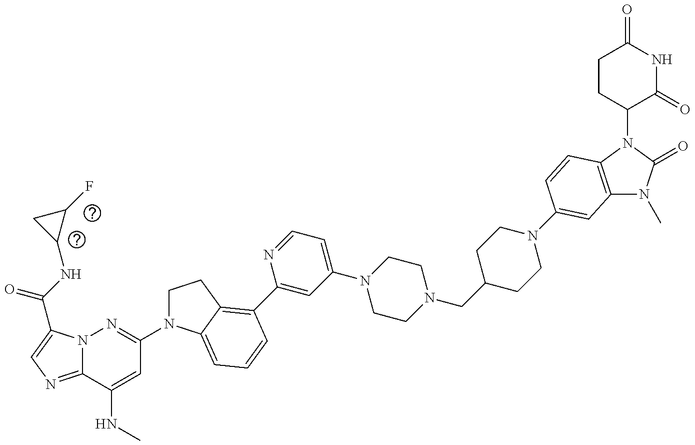 [SMILES: CNc1cc(N2CCc3c(-c4cc(N5CCN(CC6CCN(c7ccc8c(c7)n(C)c(=O)n8C7CCC(=O)NC7=O)CC6)CC5)ccn4)cccc32)nn2c(C(=O)NC3CC3F)cnc12] |
| K652 | 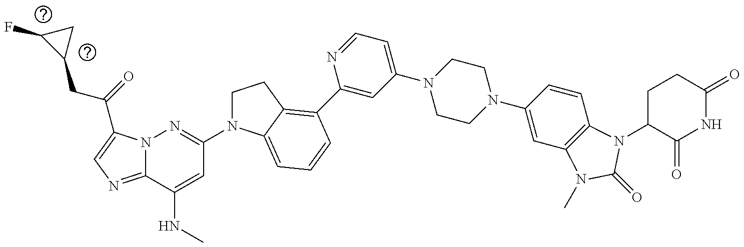 [SMILES: CNc1cc(N2CCc3c(-c4cc(N5CCN(c6ccc7c(c6)n(C)c(=O)n7C6CCC(=O)NC6=O)CC5)ccn4)cccc32)nn2c(C(=O)C[C@@H]3C[C@@H]3F)cnc12] |
| K653 | 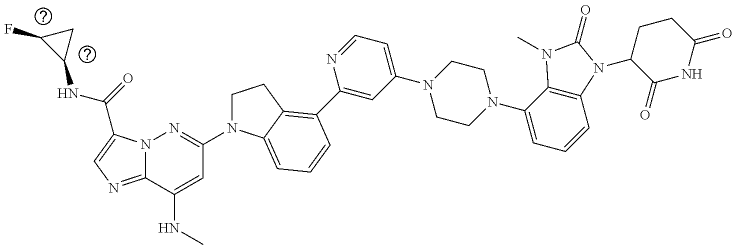 [SMILES: CNc1cc(N2CCc3c(-c4cc(N5CCN(c6cccc7c6n(C)c(=O)n7C6CCC(=O)NC6=O)CC5)ccn4)cccc32)nn2c(C(=O)N[C@@H]3C[C@@H]3F)cnc12] |
| K654 | 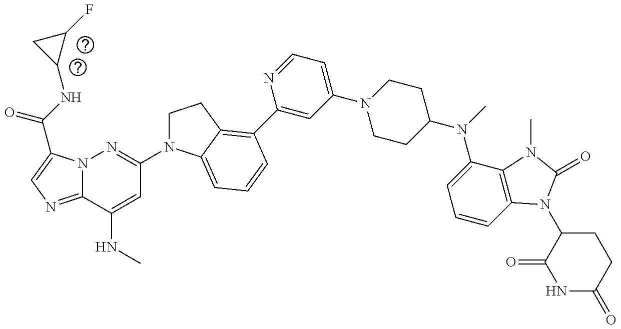 [SMILES: CNc1cc(N2CCc3c(-c4cc(N5CCC(N(C)c6cccc7c6n(C)c(=O)n7C6CCC(=O)NC6=O)CC5)ccn4)cccc32)nn2c(C(=O)NC3CC3F)cnc12] |
| K655 | 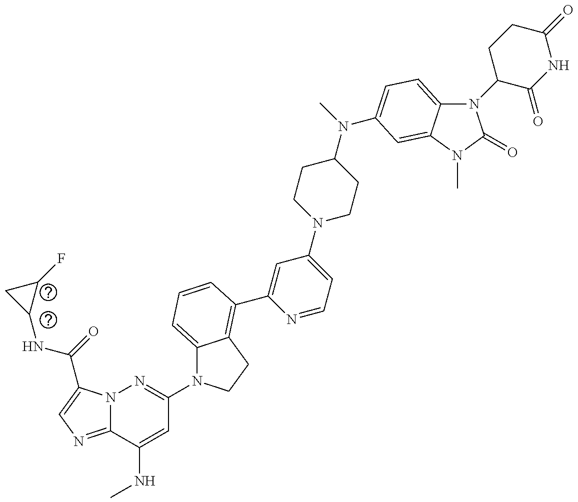 [SMILES: CNc1cc(N2CCc3c(-c4cc(N5CCC(N(C)c6ccc7c(c6)n(C)c(=O)n7C6CCC(=O)NC6=O)CC5)ccn4)cccc32)nn2c(C(=O)NC3CC3F)cnc12] |
| K656 | 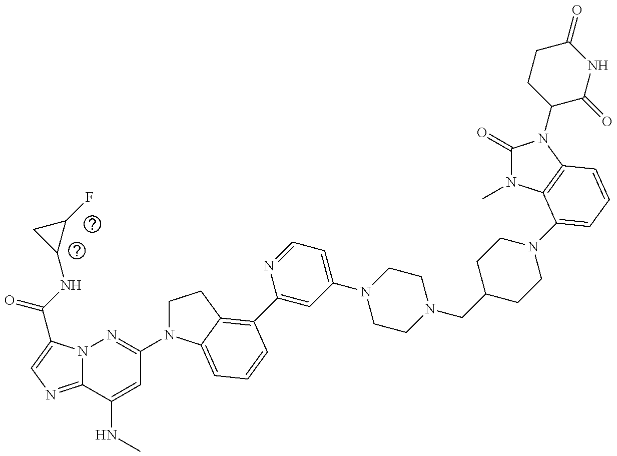 [SMILES: CNc1cc(N2CCc3c(-c4cc(N5CCN(CC6CCN(c7cccc8c7n(C)c(=O)n8C7CCC(=O)NC7=O)CC6)CC5)ccn4)cccc32)nn2c(C(=O)NC3CC3F)cnc12] |
| K657 | 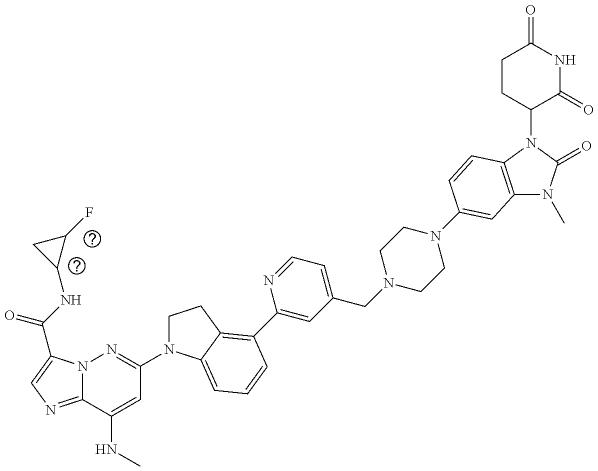 [SMILES: CNc1cc(N2CCc3c(-c4cc(CN5CCN(c6ccc7c(c6)n(C)c(=O)n7C6CCC(=O)NC6=O)CC5)ccn4)cccc32)nn2c(C(=O)NC3CC3F)cnc12] |
| K658 | 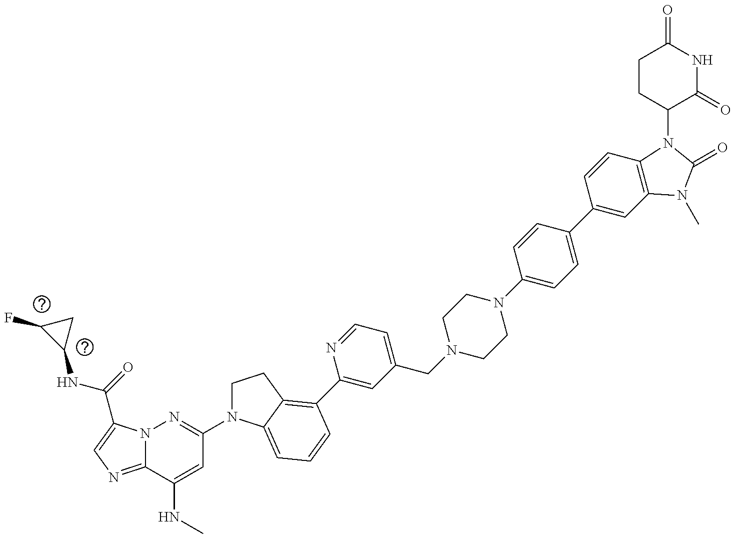 [SMILES: CNc1cc(N2CCc3c(-c4cc(CN5CCN(c6ccc(-c7ccc8c(c7)n(C)c(=O)n8C7CCC(=O)NC7=O)cc6)CC5)ccn4)cccc32)nn2c(C(=O)N[C@@H]3C[C@@H]3F)cnc12] |
| K659 | 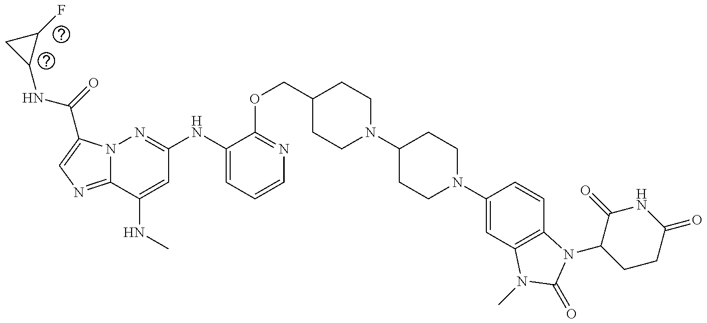 [SMILES: CNc1cc(Nc2cccnc2OCC2CCN(C3CCN(c4ccc5c(c4)n(C)c(=O)n5C4CCC(=O)NC4=O)CC3)CC2)nn2c(C(=O)NC3CC3F)cnc12] |
| K660 | 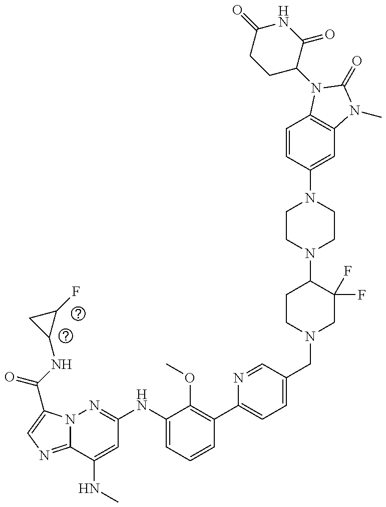 [SMILES: CNc1cc(Nc2cccc(-c3ccc(CN4CCC(N5CCN(c6ccc7c(c6)n(C)c(=O)n7C6CCC(=O)NC6=O)CC5)C(F)(F)C4)cn3)c2OC)nn2c(C(=O)NC3CC3F)cnc12] |
| K661 | 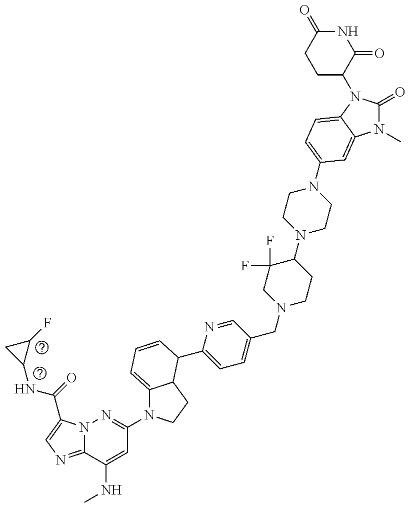 [SMILES: CNc1cc(N2CCC3C2=CC=CC3c2ccc(CN3CCC(N4CCN(c5ccc6c(c5)n(C)c(=O)n6C5CCC(=O)NC5=O)CC4)C(F)(F)C3)cn2)nn2c(C(=O)NC3CC3F)cnc12] |
| K662 | 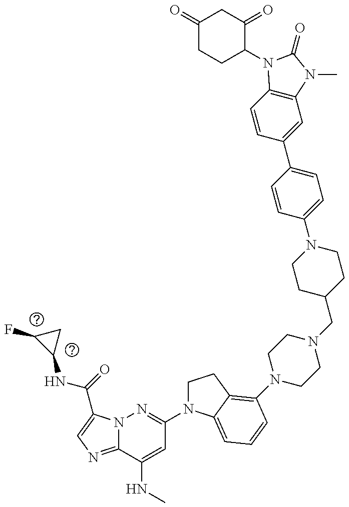 [SMILES: CNc1cc(N2CCc3c(N4CCN(CC5CCN(c6ccc(-c7ccc8c(c7)n(C)c(=O)n8C7CCC(=O)CC7=O)cc6)CC5)CC4)cccc32)nn2c(C(=O)N[C@@H]3C[C@@H]3F)cnc12] |

In some embodiments, the present invention provides a compound set forth in Table 1, above, or a pharmaceutically acceptable salt thereof.

The compounds of the present invention also encompasses, for example, the following embodiments:

In an aspect, described herein is a compound of Formula (II):

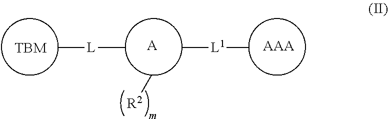

or a pharmaceutically acceptable salt thereof, wherein
TBM is a TYK binding moiety capable of binding to TYK2 protein;
L is a bivalent moiety that connects TBM to ring A and wherein:
Ring AAA is selected from:

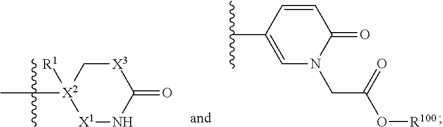

R¹⁰⁰ is C₁-C₆ alkyl or H; X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

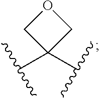 [SMILES: *C1(*)COC1]

X² is a carbon atom, a nitrogen atom, or silicon atom; X³ is a bivalent moiety selected from a covalent bond, —CR₂—, —NR—, —O—, —S—, or —Si(R)₂—; R¹ is absent, hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic; each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring A is selected from the group consisting of

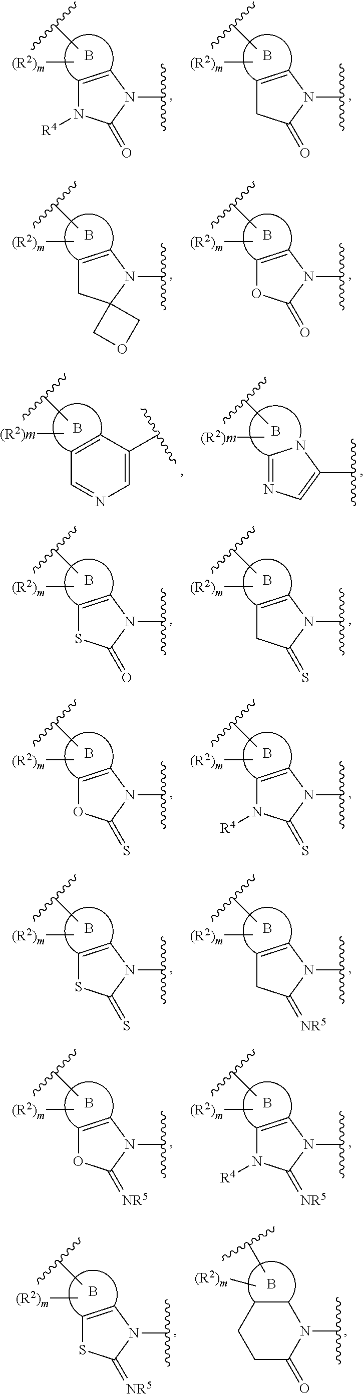

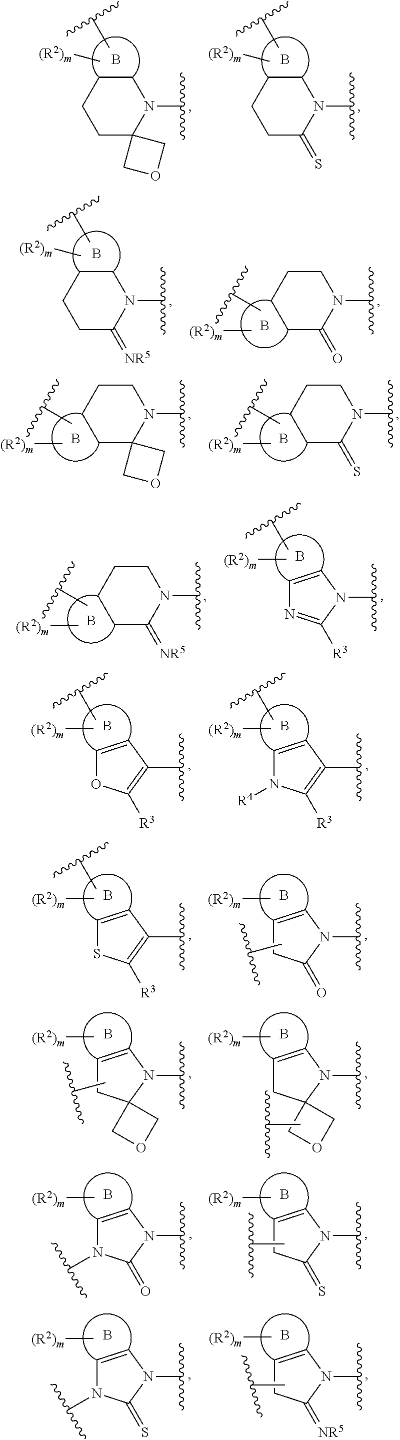

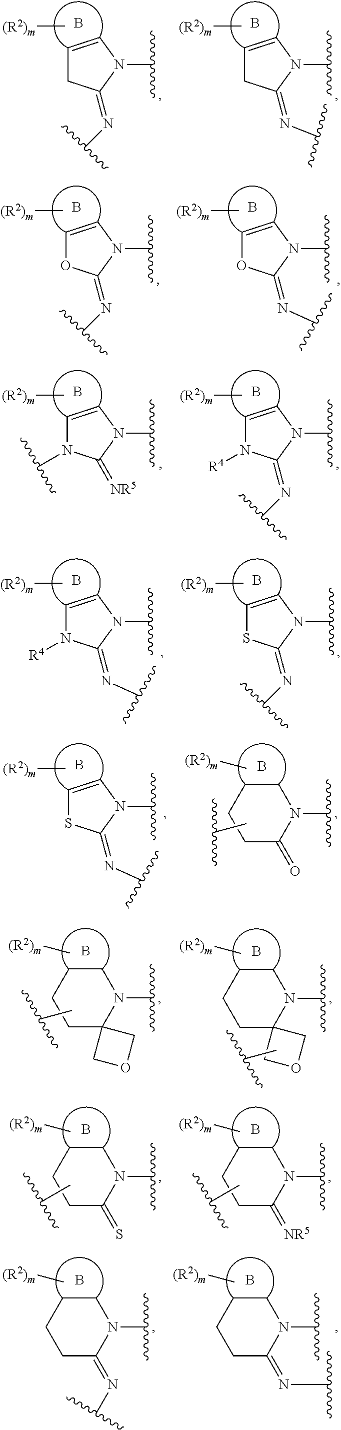

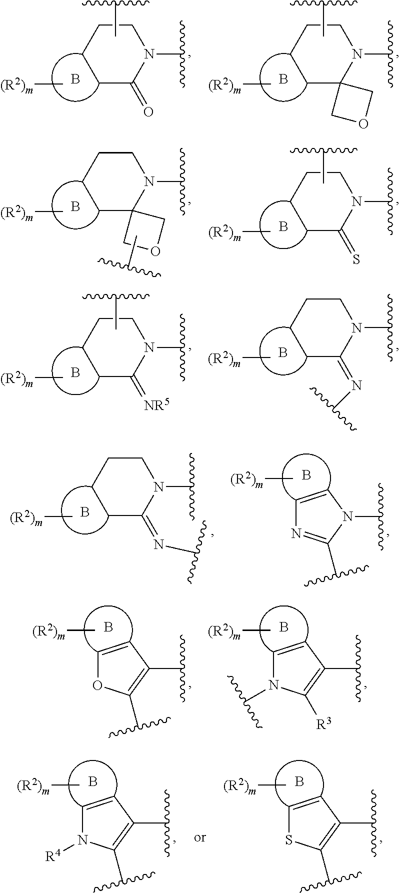

6-membered aryl optionally substituted with one or more occurrences of halogen, and null, Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur; R³ is selected from hydrogen, halogen, —OR, —N(R)₂, or —SR; each R⁴ is independently hydrogen, —R⁶, halogen, —CN, —NO₂, —OR, —SR, —NR₂, —S(O)₂R, —S(O)2NR2, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R; R⁵ is hydrogen, C₁-C₄ aliphatic, or —CN; each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur; Li is a covalent bond or a C₁-C₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—; m is 0, 1, 2, 3 or 4; and each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, ring AAA is:

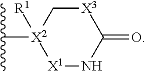

In some embodiments, ring A is:

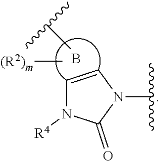

In some embodiments, ring A is:

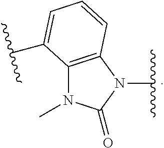 [SMILES: *c1cccc2c1n(C)c(=O)n2*]

In some embodiments, ring A is:

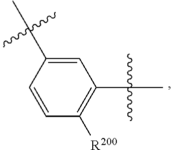

wherein R²⁰⁰ is halogen.

In some embodiments, TBM has a structure of Formula (IIAA′):

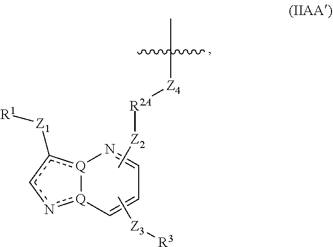

or a pharmaceutically acceptable salt thereof, wherein Q is independently at each occurrence selected from —CH— and —N— when Q is attached to only single bonds, or Q is —C═ when Q is attached to a double bond; R¹ is selected from a hydrogen, a C₁-C₆ aliphatic, Ring 1, —C₁-C₆ alkylene-Ring 1, and -Ring 1′-C₁-C₆ aliphatic; wherein each of the C₁-C₆ aliphatic, the Ring 1, the C₁-C₆ alkylene-Ring 1, and the -Ring 1′-C₁-C₆ aliphatic is independently optionally substituted with one or more of —F, —Cl, —Br, —I, and —OR^{C}; R^{2A} is selected from a covalent bond, a C₁-C₁₂ alkylene, which C₁-C₁₂ alkylene optionally comprises 1-8 heteroatoms independently selected from O, N, and S, and Ring 2A, or R^{2A} is —C₁-C₁₂ alkylene-Ring 2A-, -Ring 2A-Z₅-, or -(Ring 2A)-Z₅-(Ring 2A)-, wherein when R^{2A} is -(Ring 2A)-Z₅-(Ring 2A)- two Ring 2A may be the same or different; wherein each of the C₁-C₁₂ alkylene and the Ring 2A is independently optionally substituted with one or more of R^{K}; R³ is selected from a hydrogen and a C₁-C₆ aliphatic, which C₁-C₆ aliphatic optionally comprises 1-8 heteroatoms independently selected from O, N, and S, wherein the C₁-C₆ aliphatic is optionally substituted with one or more of R^{K}; Z₁ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z₂ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z₃ is selected from a covalent bond, —O—, —NR_{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z₄ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z₅ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S; Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S; Ring 2A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof, each R^{K} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, a C₁-C₆ aliphatic group, or two R^{K} groups attached to the same carbon or sulfur atom are optionally taken together to form ═O; each R^{C} is independently hydrogen or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S,
and

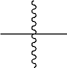

represents the point of attachment to L.

In some embodiments TBM has a structure of Formula (IIAA′):

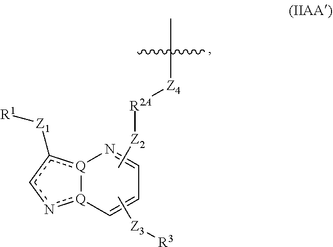

or a pharmaceutically acceptable salt thereof, wherein Q is independently at each occurrence selected from —CH— and —N— when Q is attached to only single bonds, or Q is —C═ when Q is attached to a double bond; R¹ is selected from a hydrogen, a C₁-C₆ aliphatic, Ring 1, —C₁-C₆ alkylene-Ring 1, and -Ring 1′-C₁-C₆ aliphatic; wherein each of the C₁-C₆ aliphatic, the Ring 1, the C₁-C₆ alkylene-Ring 1, and the -Ring 1′-C₁-C₆ aliphatic is independently optionally substituted with one or more of —CN, —F, —Cl, —Br, —I, and —OR^{C}; R^{2A} is selected from a covalent bond, a C₁-C₁₂ alkylene, which C₁-C₁₂ alkylene optionally comprises 1-8 heteroatoms independently selected from O, N, and S, and Ring 2A, or R^{2A} is —C₁-C₁₂ alkylene-Ring 2A-, -Ring 2A-Z₅-, or -(Ring 2A)-Z₅-(Ring 2A)-, wherein when R^{2A} is -(Ring 2A)-Z₅-(Ring 2A)- two Ring 2A may be the same or different; wherein each of the C₁-C₁₂ alkylene and the Ring 2A is independently optionally substituted with one or more of R^{K};

R³ is selected from a hydrogen and a C₁-C₆ aliphatic, which C₁-C₆ aliphatic optionally comprises 1-8 heteroatoms independently selected from O, N, and S, wherein the C₁-C₆ aliphatic is optionally substituted with one or more of R^{K}; Z₁ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z₂ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z₃ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z₄ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z₅ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 6-10 membered bicyclic saturated or partially unsaturated heterocyclyl having 1-2 heteroatoms independently selected from N, O, and S, and a 5-6 membered heteroaryl having 1-4 heteroatoms independently selected from N, O, and S; Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S; Ring 2A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof, each R^{K} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, a C₁-C₆ aliphatic group, or two R^{K} groups attached to the same carbon or sulfur atom are optionally taken together to form ═O;

each R^{C} is independently hydrogen or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S,
and

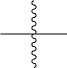

represents the point of attachment to L.

In some embodiments, TBM has a structure of Formula (IIAA′):

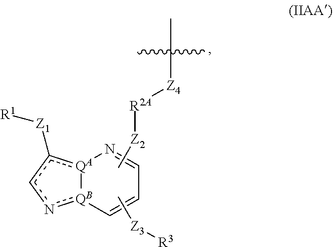

or a pharmaceutically acceptable salt thereof, wherein Q^{A} is CH and Q^{B} is N or Q^{A} is N and Q^{B} is CH;
R¹ is selected from a hydrogen, a C₁-C₆ aliphatic, Ring 1, —C₁-C₆ alkylene-Ring 1, and -Ring 1′-C₁-C₆ aliphatic; wherein each of the C₁-C₆ aliphatic, the Ring 1, the C₁-C₆ alkylene-Ring 1, and the -Ring 1′-C₁-C₆ aliphatic is independently optionally substituted with one or more of —CN, —F, —Cl, —Br, —I, and —OR^{C};
R^{2A} is selected from a covalent bond, a C₁-C₁₂ alkylene, which C₁-C₁₂ alkylene optionally comprises 1-8 heteroatoms independently selected from O, N, and S, and Ring 2A, or R^{2A} is —C₁-C₁₂ alkylene-Ring 2A-, -Ring 2A-Z₅-, or -(Ring 2A)-Z₅-(Ring 2A)-, wherein when R^{2A} is -(Ring 2A)-Z₅-(Ring 2A)- two Ring 2A may be the same or different; wherein each of the C₁-C₁₂ alkylene and the Ring 2A is independently optionally substituted with one or more of R^{K}; R³ is selected from a hydrogen and a C₁-C₆ aliphatic, which C₁-C₆ aliphatic optionally comprises 1-8 heteroatoms independently selected from O, N, and S, wherein the C₁-C₆ aliphatic is optionally substituted with one or more of R^{K}; Z₁ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(o)O—; Z₂ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(o)O—; Z₃ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(o)O—; Z₄ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(o)O—; Z₅ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 6-10 membered bicyclic saturated or partially unsaturated heterocyclyl having 1-2 heteroatoms independently selected from N, O, and S, and a 5-6 membered heteroaryl having 1-4 heteroatoms independently selected from N, O, and S; Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S; Ring 2A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof, each R^{K} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, a C₁-C₆ aliphatic group, or two R^{K} groups attached to the same carbon or sulfur atom are optionally taken together to form ═O; each R^{C} is independently hydrogen or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, and

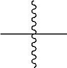

represents the point of attachment to L.

In some embodiments, TBM has a structure of Formula (IIA′), Formula (IIA″), or Formula (IIB):

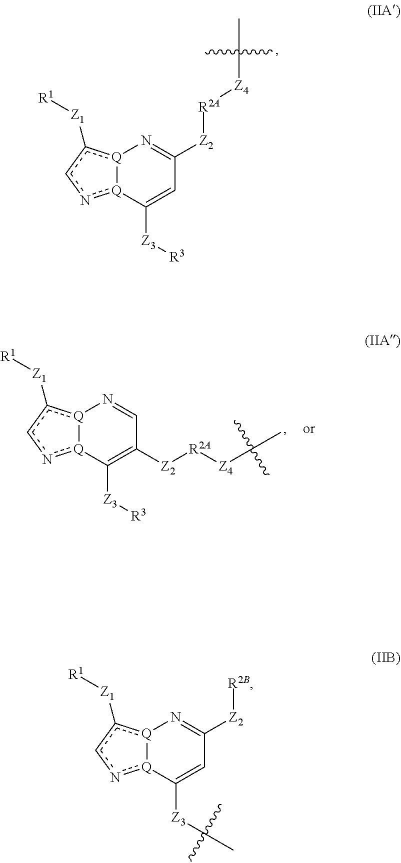

or a pharmaceutically acceptable salt thereof,
wherein Q is independently at each occurrence selected from —CH— and —N— when Q is attached to only single bonds, or Q is —C═ when Q is attached to a double bond;
R¹ is selected from a hydrogen, a C₁-C₆ aliphatic, Ring 1, —C₁-C₆ alkylene-Ring 1, and -Ring 1′-C₁-C₆ aliphatic; wherein each of the C₁-C₆ aliphatic, the C₁-C₆ alkylene-Ring 1, and the -Ring 1′-C₁-C₆ aliphatic is independently optionally substituted with one or more of —F, —Cl, —Br, —I, and —OR^{C};
R^{2A} is selected from a covalent bond, a C₁-C₁₂ alkylene, which C₁-C₁₂ alkylene optionally comprises 1-8 heteroatoms independently selected from O, N, and S, and Ring 2A, or R^{2A} is —C₁-C₁₂ alkylene-Ring 2A-, -Ring 2A-Z₅-, or -(Ring 2A)-Z₅-(Ring 2A)-, wherein when R^{2A} is -(Ring 2A)-Z₅-(Ring 2A)- two Ring 2A may be the same or different; wherein each of the C₁-C₁₂ alkylene and the Ring 2A is independently optionally substituted with one or more of R^{K};
R^{2B} is selected from a hydrogen, a C₁-C₆ aliphatic, and Ring 2B, or R^{2B} is —C₁-C₆ alkylene-Ring 2B, -Ring 2B′-C₁-C₆ aliphatic, -Ring 2B′-Z₅-Ring 2B wherein each of the C₁-C₆ aliphatic, the C₁-C₆ alkylene, the Ring 2B, the Ring 2B′ is independently optionally substituted with one or more of R^{K};
R³ is selected from a hydrogen and a C₁-C₆ aliphatic, which C₁-C₆ aliphatic optionally comprises 1-8 heteroatoms independently selected from O, N, and S, wherein the C₁-C₆ aliphatic is optionally substituted with one or more of R^{K};
Z₁ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₂ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₃ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₄ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₅ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S;
Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S;
Ring 2A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;

Ring 2B is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S; and
Ring 2B′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
each R^{K} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, a C₁-C₆ aliphatic group, or two R^{K} groups attached to the same carbon or sulfur atom are optionally taken together to form ═O;

each R^{C} is independently hydrogen or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S,

and

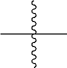

represents the point of attachment to L.

In some embodiments, TBM has a structure of Formula (IIA′), Formula (IIA″), or Formula (IIB):

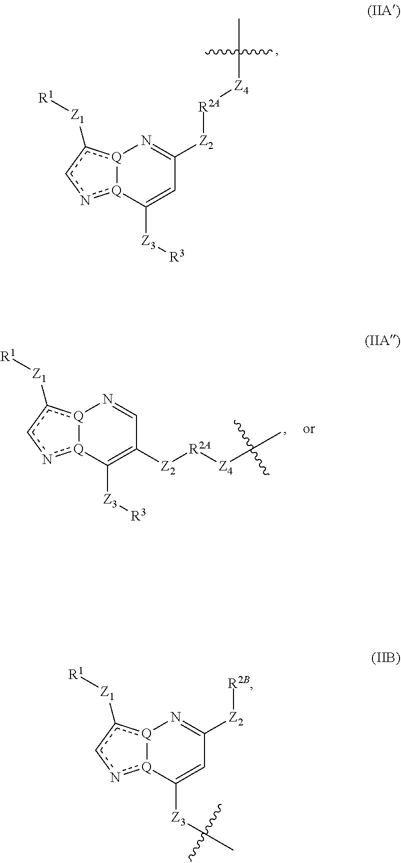

or a pharmaceutically acceptable salt thereof,
wherein Q is independently at each occurrence selected from —CH— and —N— when Q is attached to only single bonds, or Q is —C═ when Q is attached to a double bond;
R¹ is selected from a hydrogen, a C₁-C₆ aliphatic, Ring 1, —C₁-C₆ alkylene-Ring 1, and -Ring 1′-C₁-C₆ aliphatic; wherein each of the C₁-C₆ aliphatic, the C₁-C₆ alkylene-Ring 1, and the -Ring 1′-C₁-C₆ aliphatic is independently optionally substituted with one or more of —CN, —F, —Cl, —Br, —I, and —OR^{C};
R^{2A} is selected from a covalent bond, a C₁-C₁₂ alkylene, which C₁-C₁₂ alkylene optionally comprises 1-8 heteroatoms independently selected from O, N, and S, and Ring 2A, or R^{2A} is —C₁-C₁₂ alkylene-Ring 2A-, -Ring 2A-Z₅-, or -(Ring 2A)-Z₅-(Ring 2A)-, wherein when R^{2A} is -(Ring 2A)-Z₅-(Ring 2A)- two Ring 2A may be the same or different; wherein each of the C₁-C₁₂ alkylene and the Ring 2A is independently optionally substituted with one or more of R^{K};
R^{2B} is selected from a hydrogen, a C₁-C₆ aliphatic, and Ring 2B, or R^{2B} is —C₁-C₆ alkylene-Ring 2B, -Ring 2B′-C₁-C₆ aliphatic, -Ring 2B′-Z₅-Ring 2B wherein each of the C₁-C₆ aliphatic, the C₁-C₆ alkylene, the Ring 2B, the Ring 2B′ is independently optionally substituted with one or more of R^{K};
R³ is selected from a hydrogen and a C₁-C₆ aliphatic, which C₁-C₆ aliphatic optionally comprises 1-8 heteroatoms independently selected from O, N, and S, wherein the C₁-C₆ aliphatic is optionally substituted with one or more of R^{K}; Z₁ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z₂ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z₃ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z₄ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z₅ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 6-10 membered bicyclic saturated or partially unsaturated heterocyclyl having 1-2 heteroatoms independently selected from N, O, and S, and a 5-6 membered heteroaryl having 1-4 heteroatoms independently selected from N, O, and S; Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S; Ring 2A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof, Ring 2B is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S; and
Ring 2B′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof, each R^{K} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, a C₁-C₆ aliphatic group, or two R^{K} groups attached to the same carbon or sulfur atom are optionally taken together to form ═O; each R^{C} is independently hydrogen or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S,
and

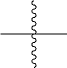

represents the point of attachment to L.

In some embodiments, TBM has a structure of Formula (IIA′-1-1), Formula (IIA″-1-1), or Formula (IIB-1-1):

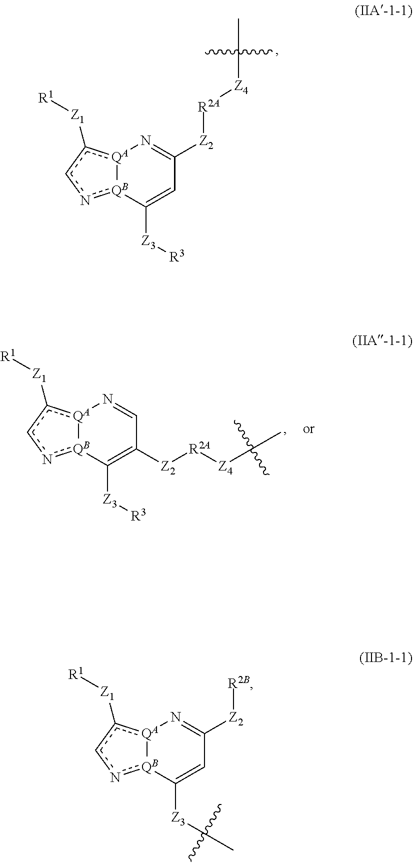

or a pharmaceutically acceptable salt thereof,
wherein Q^{A} is CH and Q^{B} is N or Q^{A} is N and Q^{B} is CH;
R¹ is selected from a hydrogen, a C₁-C₆ aliphatic, Ring 1, —C₁-C₆ alkylene-Ring 1, and -Ring 1′-C₁-C₆ aliphatic; wherein each of the C₁-C₆ aliphatic, the C₁-C₆ alkylene-Ring 1, and the -Ring 1′-C₁-C₆ aliphatic is independently optionally substituted with one or more of —CN, —F, —Cl, —Br, —I, and —OR^{C}.
R^{2A} is selected from a covalent bond, a C₁-C₁₂ alkylene, which C₁-C₁₂ alkylene optionally comprises 1-8 heteroatoms independently selected from O, N, and S, and Ring 2A, or R^{2A} is —C₁-C₁₂ alkylene-Ring 2A-, -Ring 2A-Z₅-, or -(Ring 2A)-Z₅-(Ring 2A)-, wherein when R^{2A} is -(Ring 2A)-Z₅-(Ring 2A)- two Ring 2A may be the same or different; wherein each of the C₁-C₁₂ alkylene and the Ring 2A is independently optionally substituted with one or more of R^{K};
R^{2B} is selected from a hydrogen, a C₁-C₆ aliphatic, and Ring 2B, or R^{2B} is —C₁-C₆ alkylene-Ring 2B, -Ring 2B′-C₁-C₆ aliphatic, -Ring 2B′-Z₅-Ring 2B wherein each of the C₁-C₆ aliphatic, the C₁-C₆ alkylene, the Ring 2B, the Ring 2B′ is independently optionally substituted with one or more of R^{K};
R³ is selected from a hydrogen and a C₁-C₆ aliphatic, which C₁-C₆ aliphatic optionally comprises 1-8 heteroatoms independently selected from O, N, and S, wherein the C₁-C₆ aliphatic is optionally substituted with one or more of R^{K};
Z₁ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₂ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₃ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₄ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₅ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 6-10 membered bicyclic saturated or partially unsaturated heterocyclyl having 1-2 heteroatoms independently selected from N, O, and S, and a 5-6 membered heteroaryl having 1-4 heteroatoms independently selected from N, O, and S;
Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S;
Ring 2A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
Ring 2B is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S; and
Ring 2B′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
each R^{K} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, a C₁-C₆ aliphatic group, or two R^{K} groups attached to the same carbon or sulfur atom are optionally taken together to form ═O; each R^{C} is independently hydrogen or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S,
and

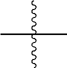

represents the point of attachment to L.

In some embodiments, TBM has a structure of Formula (IIBB′-4), Formula (IIBB′-4-1), Formula (IIA′A′-4), or Formula (IIA′A′-4-1):

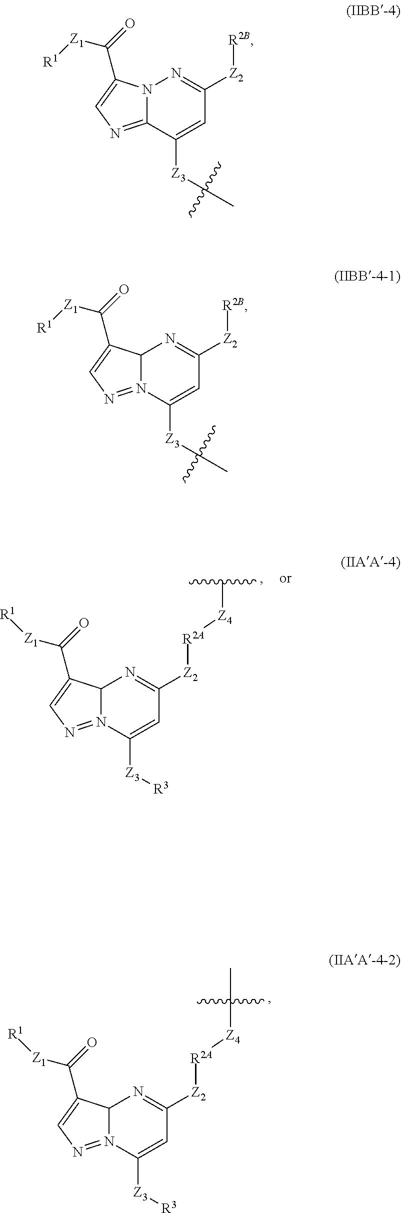

wherein R¹ is selected from a hydrogen, Ring 1, -Ring 1′-C₁-C₆ aliphatic, wherein the Ring 1 or the -Ring 1′-C₁-C₆ aliphatic is optionally substituted with one or more of —F, —Cl, —Br, —I, and —OR^{C};
Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S;
Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S;
R^{2A} is selected from a covalent bond, a C₁-C₁₂ alkylene, which C₁-C₁₂ alkylene optionally comprises 1-8 heteroatoms independently selected from O, N, and S, and Ring 2A, wherein each of the C₁-C₁₂ alkylene and the Ring 2A is optionally substituted with one or more of R^{K};
R^{2B} is selected from a hydrogen, a C₁-C₆ aliphatic, and Ring 2B, wherein the C₁-C₆ aliphatic and the Ring 2B is optionally substituted with one or more of R^{K};
Ring 2B is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
R³ is hydrogen or a C₁-C₃ aliphatic;
Z₁ is selected from a covalent bond, —O—, —NR^{C}—, —C(O)—, —CR^{C}₂—, —OC(O)—, —C(O)O—, —N(R^{C})C(O)—, and —C(O)N(R^{C})—;
Z₂ is selected from a covalent bond, —O—, —NR^{C}—, —C(O)—, —CR^{C}₂—, —OC(O)—, —C(O)O—, —N(R^{C})C(O)—, and —C(O)N(R^{C})—;
Z₃ is selected from a covalent bond, —O—, —NR^{C}—, —C(O)—, —CR^{C}₂—, —OC(O)—, —C(O)O—, —N(R^{C})C(O)—, and —C(O)N(R^{C})—;
Z₄ is selected from a covalent bond, —O—, —NR^{C}—, —C(O)—, —CR^{C}₂—, —OC(O)—, —C(O)O—, —N(R^{C})C(O)—, and —C(O)N(R^{C})—;
each R^{K} is independently hydrogen, —F, —Cl, —Br, —I, —OH, —O—(C₁-C₆ aliphatic), —NH₂, —NH—(C₁-C₆ aliphatic), —N(C₁-C₆ aliphatic)₂, —N(C₁-C₆ aliphatic)-OH, —N(C₁-C₆ aliphatic)-O—(C₁-C₆ aliphatic), —CN, —C(O)—C₁-C₆ aliphatic, —CHO, —CO₂H, —CO₂(C₁-C₆ aliphatic), —C(O)—NH₂, —C(O)—N(C₁-C₆ aliphatic)₂, —N(C₁-C₆ aliphatic)-C(O)—(C₁-C₆ aliphatic), —CF₃, —O—CF₃, a C₁-C₆ aliphatic group, or two R^{K} groups attached to the same carbon atom are optionally taken together to form ═O;
R^{C} is hydrogen or a C₁-C₆ aliphatic;
and

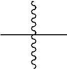

represents the point of attachment to L.

In some embodiments, TBM has a structure of Formula (IIBB′-3) or Formula (IIA′A′-3):

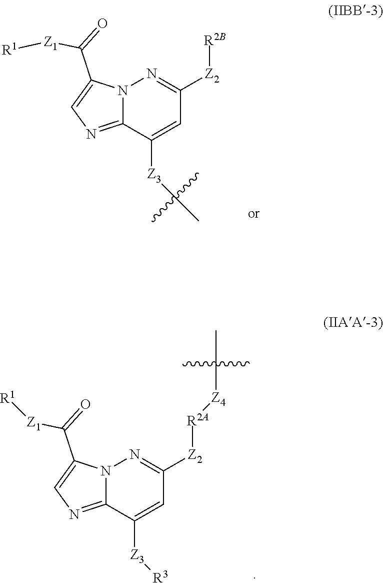

In some embodiments, TBM has a structure of Formula (IIBB′-2) or Formula (IIA′A′-2):

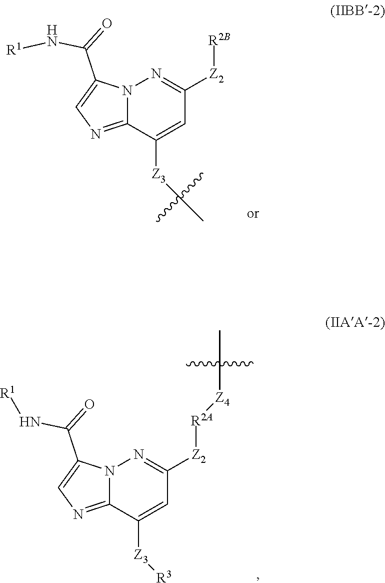

wherein R¹ is H, —C(CH₃)₂—CN,

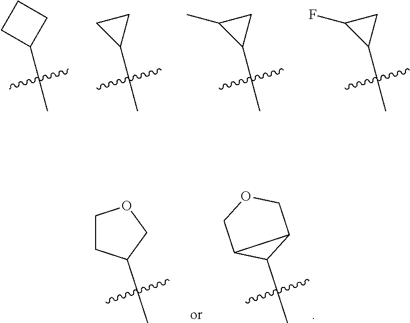

In some embodiments, the compound is represented by Formula (II-AB):

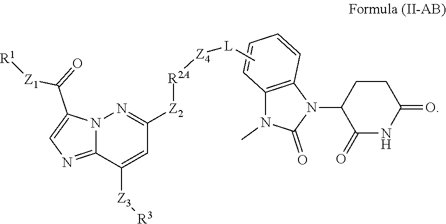

In some embodiments, the compound is represented by Formula (II-AB-I):

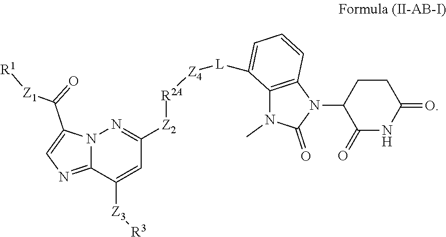

In some embodiments, in the compound is represented by Formula (II-AB-II):

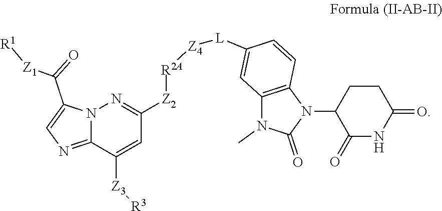

In some embodiments, the compound is represented by Formula (II-AB-III):

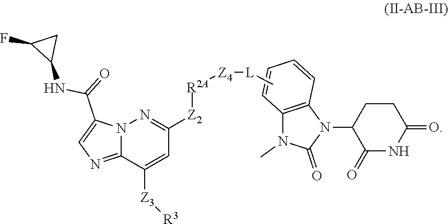

In some embodiments, the compound is represented by Formula (II-AB-III):

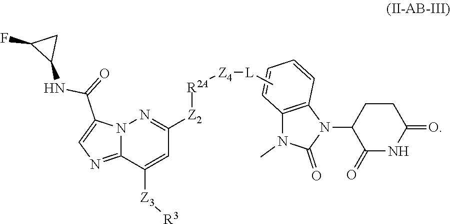

In some embodiments, the compound is represented by Formula (II-AB-III):

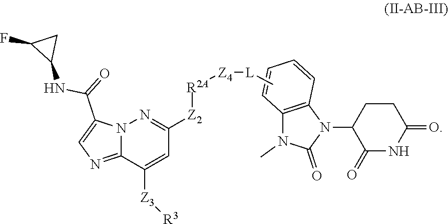

In some embodiments, R¹ is:
H, —C(CH₃)₂—CN,

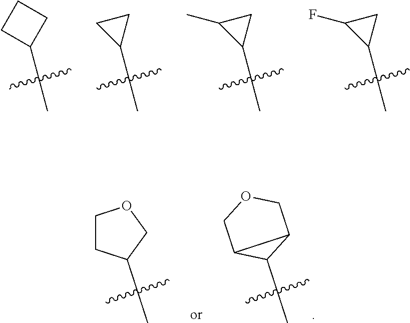

In some embodiments, R^{2B} is selected from a hydrogen, a C₁-C₆ aliphatic, and Ring 2B, wherein the C₁-C₆ aliphatic and the Ring 2B is optionally substituted with one or more of R^{K}.

In some embodiments, Ring 2B is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S.

In some embodiments, R¹ is selected from a hydrogen, Ring 1, -Ring 1′-C₁-C₆ aliphatic, wherein the Ring 1 or the -Ring 1′-C₁-C₆ aliphatic is optionally substituted with one or more of —F, —Cl, —Br, —I, and —OR^{C}.

In some embodiments, R^{2A} is selected from a covalent bond, a C₁-C₁₂ alkylene, which C₁-C₁₂ alkylene optionally comprises 1-8 heteroatoms independently selected from O, N, and S, and Ring 2A, wherein each of the C₁-C₁₂ alkylene and the Ring 2A is optionally substituted with one or more of R^{K}.

In some embodiments, R³ is selected from a hydrogen and a C₁-C₃ aliphatic, the C₁-C₃ aliphatic optionally comprises 1-8 heteroatoms independently selected from O, N, and S, and the C₁-C₃ aliphatic is optionally substituted with one or more of R^{K}.

In some embodiments, Z₁ is selected from a covalent bond, —O—, —NR^{C}—, —C(O)—, —CR^{C}₂—, —OC(O)—, —C(O)O—, —N(R^{C})C(O)—, and —C(O)N(R^{C})—.

In some embodiments, Z₂ is selected from a covalent bond, —O—, —NR^{C}—, —C(O)—, —CR^{C}₂—, —OC(O)—, —C(O)O—, —N(R^{C})C(O)—, and —C(O)N(R^{C})—.

In some embodiments, Z₂ is —NH—.

In some embodiments, Z₂ is a covalent bond.

In some embodiments, R^{C} is hydrogen or a C₁-C₆ aliphatic.

In some embodiments, R³ is an aliphatic C₁-C₄ hydrocarbon.

In some embodiments, R³ is —CH₃.

In some embodiments, the Z₂-R^{2B} group is selected from:

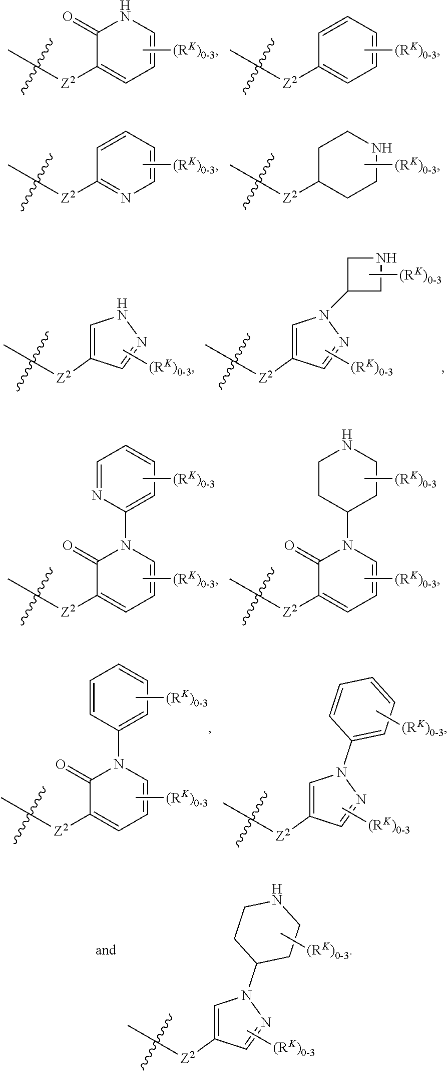

In some embodiments, Z₃ is selected from a covalent bond, —O—, —NR^{C}—, —C(O)—, —CR^{C}₂—, —OC(O)—, —C(O)O—, —N(R^{C})C(O)—, and —C(O)N(R^{C})—.

In some embodiments, Z₃ is —NH—.

In some embodiments, Z₄ is selected from a covalent bond, —O—, —NR^{C}—, —C(O)—, —CR^{C}₂—, —OC(O)—, —C(O)O—, —N(R^{C})C(O)—, and —C(O)N(R^{C})—.

In some embodiments, Z₄ is a covalent bond, —O—, —C(O)—, —NH—, or —C(O)—N(CH₃).

In some embodiments, Z₅ is selected from a covalent bond, —O—, —NR^{C}—, —C(O)—, —CR^{C}₂—, —OC(O)—, —C(O)O—, —N(R^{C})C(O)—, and —C(O)N(R^{C})—.

In some embodiments, Z₅ is a covalent bond or —O—.

In some embodiments, Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S.

In some embodiments, Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S.

In some embodiments, Ring 2A is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-3 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof.

In some embodiments, each R^{K} is independently hydrogen, —F, —Cl, —Br, —I, —OH, —O—(C₁-C₆ aliphatic), —NH₂, —NH—(C₁-C₆ aliphatic), —N(C₁-C₆ aliphatic)₂, —N(C₁-C₆ aliphatic)-OH, —N(C₁-C₆ aliphatic)-O—(C₁-C₆ aliphatic), —CN, —C(O)—C₁-C₆ aliphatic, —CHO, —CO₂H, —CO₂(C₁-C₆ aliphatic), —C(O)—NH₂, —C(O)—N(C₁-C₆ aliphatic)₂, —N(C₁-C₆ aliphatic)-C(O)—(C₁-C₆ aliphatic), —CF₃, —O—CF₃, a C₁-C₆ aliphatic group, or two R^{K} groups attached to the same carbon atom are optionally taken together to form ═O.

In some embodiments, -Z₃- is covalent bond and R³ is hydrogen.

In some embodiments, -Z₃- is —NR^{C}— and R³ is —CH₃.

In some embodiments, TBM has a structure of Formula (IIBB′-1) or Formula (IIA′A′-1):

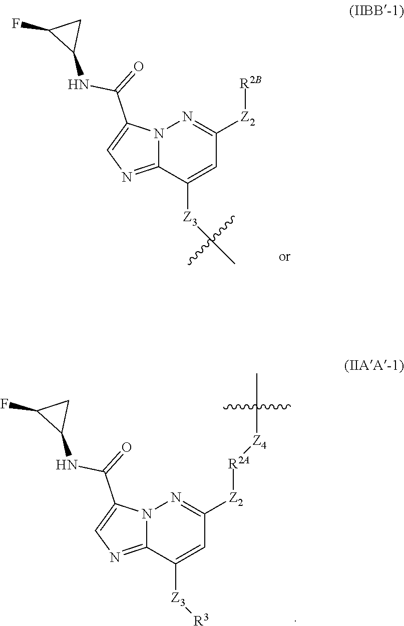

In some embodiments, Z₂ is —NH— and R^{2B} is Ring 2B.

In some embodiments, Ring 2B is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S.

In some embodiments, TBM has a structure of Formula (IIA′-2-1):

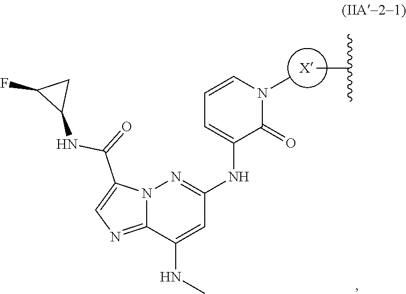

wherein Ring X′ is a ring selected from phenyl, a 4 to 7-membered saturated or partially unsaturated carbocyclyl or heterocyclyl with 1-3 heteroatoms independently selected from N, O, and S, and a 5 to 6-membered heteroaryl with 1-4 heteroatoms independently selected from N, O, and S, and each of which is optionally substituted with one or more of R^{K}.

In some embodiments, TBM has a structure of Formula (IIA′-2-a):

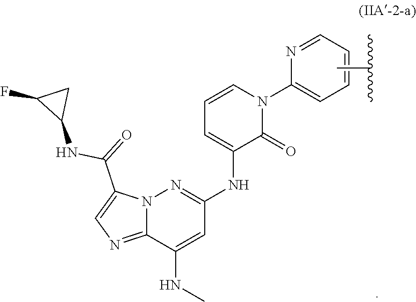

In some embodiments, TBM has a structure of Formula (IIA′-2-2):

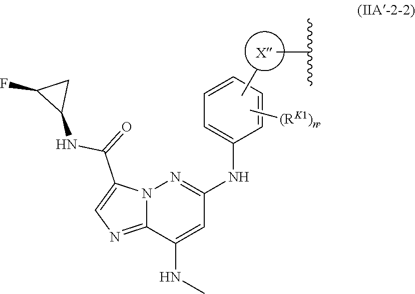

wherein
each R^{K1} is independently hydrogen, halo, C₁-C₆ aliphatic, halo substituted C₁-C₆ aliphatic, or —O—(C₁-C₆ aliphatic), Ring X″ is a ring selected from phenyl, a 4 to 7-membered saturated or partially unsaturated carbocyclyl or heterocyclyl with 1-3 heteroatoms independently selected from N, O, and S, and a 5 to 6-membered heteroaryl with 1-4 heteroatoms independently selected from N, O, and S, and each of which is optionally substituted with one or more of R^{K}, and n is 0-6.

In some embodiments, TBM has a structure of Formula (IIA′-2-2a):

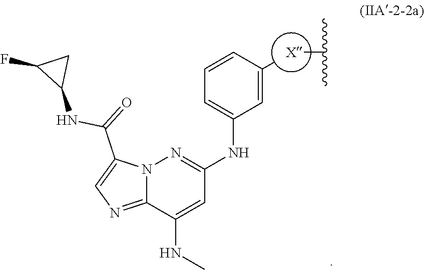

In some embodiments, TBM has a structure of Formula (IIA′-2-b)

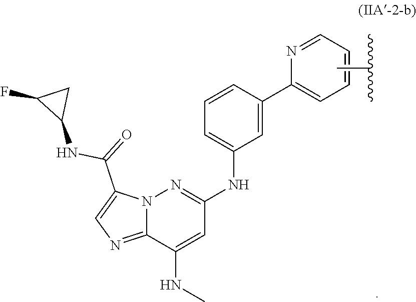

In some embodiments, TBM has a structure of Formula (IIA′-2-3):

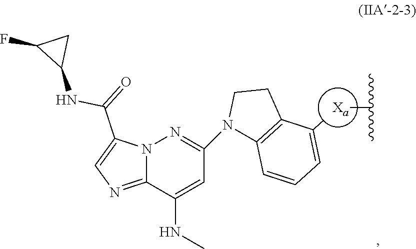

wherein Ring Xa is a ring selected from phenyl, a 4 to 7-membered saturated or partially unsaturated carbocyclyl or heterocyclyl with 1-3 heteroatoms independently selected from N, O, and S, and a 5 to 6-membered heteroaryl with 1-4 heteroatoms independently selected from N, O, and S, and each of which is optionally substituted with one or more of R^{K}.

In some embodiments, TBM has a structure of Formula (IIA′-2-3a):

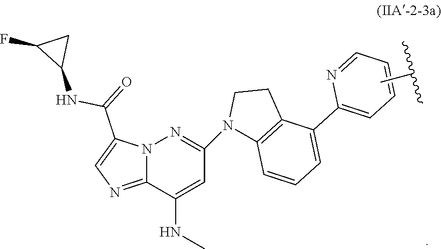

In some embodiments, TBM has a structure of Formula (IIA′-2-4)

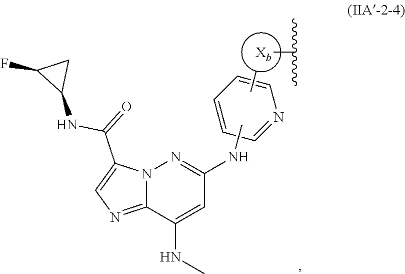

wherein Ring X_{b} is a ring selected from phenyl, a 4 to 7-membered saturated or partially unsaturated carbocyclyl or heterocyclyl with 1-3 heteroatoms independently selected from N, O, and S, and a 5 to 6-membered heteroaryl with 1-4 heteroatoms independently selected from N, O, and S, and each of which is optionally substituted with one or more of R^{K}.

In some embodiments, TBM has a structure of Formula (IIA′-2-1)

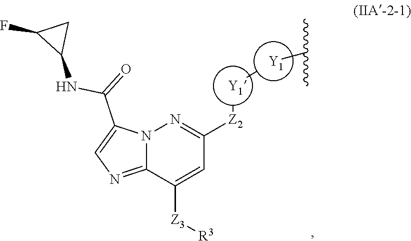

wherein Y1 and Y1′ are each independently an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S.

In some embodiments, TBM has a structure of Formula (IIIAA) or Formula (IIIBB):

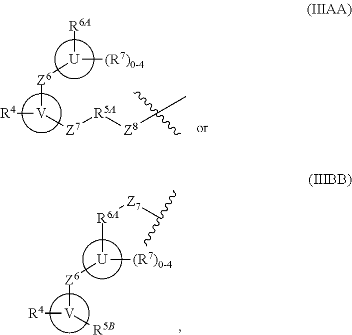

or a pharmaceutically acceptable salt thereof, U is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S; V is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S; R⁴ is hydrogen, —F, —Cl, —Br, —I, —OH, —O—(C₁-C₆ aliphatic), —NH₂, —NH—(C₁-C₆ aliphatic), —N(C₁-C₆ aliphatic)₂, —CN, —C(O)—C₁-C₆ aliphatic, —CO₂H, —CO₂(C₁-C₆ aliphatic), —C(O)—NH₂, —C(O)—NH(C₁-C₆ aliphatic), —C(O)—N(C₁-C₆ aliphatic)₂, —C(O)—(C₁-C₆ aliphatic), —SO₂(C₁-C₆ aliphatic), —SO₂—N(C₁-C₆ aliphatic)₂, —S(O)—C₁-C₆ aliphatic, —CD₃, —CF₃, or —O—CF₃; R^{5A} is selected from a covalent bond and Ring 5A, wherein the Ring 5A is optionally substituted with one or more R; R^{6A} is selected from a hydrogen, Ring 6A, and -Ring 6A′-Ring 6A, wherein the Ring 6A and the Ring 6A′ is independently optionally substituted with one or more R; R^{5B} is selected from a hydrogen, Ring 5B, and -Ring 5B′-Ring 5B, wherein the Ring 5B and the Ring 5B′ is independently optionally substituted with one or more R; R^{6B} is selected from a covalent bond and Ring 6B, wherein the Ring 6B is optionally substituted with one or more R⁸; Ring 5A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof, Ring 6A is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S; Ring 6A′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S; Ring 5B is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;

Ring 5B′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S; Ring 6B is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof, Z⁶ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z⁷ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z⁸ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; each R⁷ is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—NH(C₁-C₆ aliphatic), —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CD₃, —CF₃, or —O—CF₃; each R⁸ is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—NH(C₁-C₆ aliphatic), —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CD₃, —CF₃, or —O—CF₃; each R^{C} is independently hydrogen or an optionally substituted C₁-C₆ aliphatic group,

and

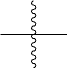

represents the point of attachment to L.

In some embodiments, TBM has a structure of Formula (IIIAA) or Formula (IIIBB):

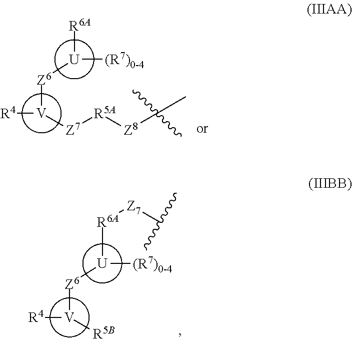

or a pharmaceutically acceptable salt thereof,
U is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
V is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
R⁴ is hydrogen, —F, —Cl, —Br, —I, —OH, —O—(C₁-C₆ aliphatic), —NH₂, —NH—(C₁-C₆ aliphatic), —N(C₁-C₆ aliphatic)₂, —CN, —C(O)—C₁-C₆ aliphatic, —CO₂H, —CO₂(C₁-C₆ aliphatic), —C(O)—NH₂, —C(O)—NH(C₁-C₆ aliphatic), —C(O)—N(C₁-C₆ aliphatic)₂, —C(O)—(C₁-C₆ aliphatic), —SO₂(C₁-C₆ aliphatic), —SO₂—N(C₁-C₆ aliphatic)₂, —S(O)—C₁-C₆ aliphatic, —CD₃, —CF₃, or —O—CF₃;
R^{5A} is selected from a covalent bond and Ring 5A, wherein the Ring 5A is optionally substituted with one or more R⁸; R^{6A} is selected from a hydrogen, Ring 6A, and -Ring 6A′-Ring 6A, wherein the Ring 6A and the Ring 6A′ is independently optionally substituted with one or more R⁸; R^{5B} is selected from a hydrogen, Ring 5B, and -Ring 5B′-Ring 5B, wherein the Ring 5B and the Ring 5B′ is independently optionally substituted with one or more R⁸; R^{6B} is selected from a covalent bond and Ring 6B, wherein the Ring 6B is optionally substituted with one or more R⁸; Ring 5A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof, Ring 6A is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S; Ring 6A′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S; Ring 5B is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S; Ring 5B′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S; Ring 6B is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof, Z⁶ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z⁷ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z⁸ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; each R⁷ is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—NH(C₁-C₆ aliphatic), —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CD₃, —CF₃, or —O—CF₃; each R⁸ is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—NH(C₁-C₆ aliphatic), —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CD₃, —CF₃, or —O—CF₃; each R^{C} is independently hydrogen or an optionally substituted C₁-C₆ aliphatic group,
and

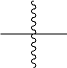

represents the point of attachment to L,
with the proviso that when TBM has a structure of Formula (IIIAA) and Z⁶ is —NH—, —Z⁶—U— is not

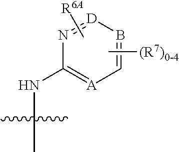

(ii) when TBM has a structure of Formula (IIIAA), R^{5A} is Ring 5A, and Z⁷ is —NH—, —Z⁶—V—Z⁷ is not

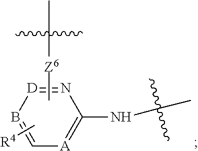

and
(iii) when TBM has a structure of Formula (IIIBB), Z⁶ is —NH—, —V—Z⁶— is not

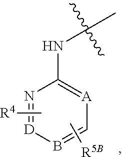

wherein A, B, D are independently —C═ or —NR^{AB}—,
R^{AB} is selected from hydrogen, halogen, hydroxyl, amino, cyano, nitro, CONR^{AB1}R^{AB2}, optionally substituted C1-C8 aliphatic, and optionally substituted 3-10 membered carbocyclyl, wherein
R^{AB1} and R^{AB2} are independently selected from hydrogen, optionally substituted C₁-C₈ aliphatic, optionally substituted C₂-C₈ alkenyl, optionally substituted C₂-C₈ alkynyl, optionally substituted 3-10 membered carbocyclyl, and optionally substituted 4-10 membered heterocyclyl, or R^{AB1} and R^{AB2} together with the atom or atoms to which they are connected form a 3-20 membered carbocyclyl ring or 4-20 membered heterocyclyl ring.

In some embodiments, U is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 5-6 membered heteroaryl having 1-4 heteroatoms independently selected from N, O, and S.

In some embodiments, V is an optionally substituted ring selected from a 5-6 membered heteroaryl having 1-4 heteroatoms independently selected from N, O, and S, and an 8-10 membered bicyclic heteroaryl having 1-5 heteroatoms independently selected from N, O, and S.

In some embodiments, TBM has a structure of Formula (IIIA) or Formula (IIIB):

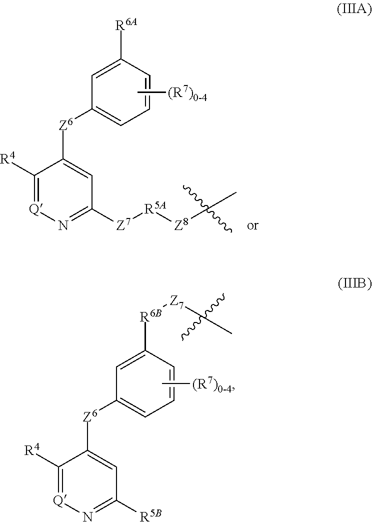

or a pharmaceutically acceptable salt thereof,
wherein Q′ is selected from —CH═ and —N═; R⁴ is hydrogen, —F, —Cl, —Br, —I, —OH, —O—(C₁-C₆ aliphatic), —NH₂, —NH—(C₁-C₆ aliphatic), —N(C₁-C₆ aliphatic)₂, —CN, —C(O)—C₁-C₆ aliphatic, —CO₂H, —CO₂(C₁-C₆ aliphatic), —C(O)—NH₂, —C(O)—NH(C₁-C₆ aliphatic), —C(O)—N(C₁-C₆ aliphatic)₂, —C(O)—(C₁-C₆ aliphatic), —SO₂(C₁-C₆ aliphatic), —SO₂—N(C₁-C₆ aliphatic)₂, —S(O)—C₁-C₆ aliphatic, —CD₃, —CF₃, or —O—CF₃; R^{5A} is selected from a covalent bond and Ring 5A, wherein the Ring 5A is optionally substituted with one or more R⁷; R^{6A} is selected from a hydrogen, Ring 6A, and -Ring 6A′-Ring 6A, wherein the Ring 6A and the Ring 6A′ is independently optionally substituted with one or more R⁷; R^{5B} is selected from a hydrogen, Ring 5B, and -Ring 5B′-Ring 5B, wherein the Ring 5B and the Ring 5B′ is independently optionally substituted with one or more R⁷; R^{6B} is selected from a covalent bond and Ring 6B, wherein the Ring 6B is optionally substituted with one or more R⁷; Ring 5A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof, Ring 6A is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S; Ring 6A′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S; Ring 5B is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S; Ring 5B′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S; Ring 6B is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof; Z⁶ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z⁷ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z⁸ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; each R⁷ is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—NH(C₁-C₆ aliphatic), —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CD₃, —CF₃, or —O—CF₃; each R^{C} is independently hydrogen or an optionally substituted C₁-C₆ aliphatic group,
and

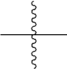

represents the point of attachment to L.

In some embodiments, TBM has a structure of Formula (IIIA-1), Formula (IIIB-1), Formula (IIIA-2), or Formula (IIIB-2):

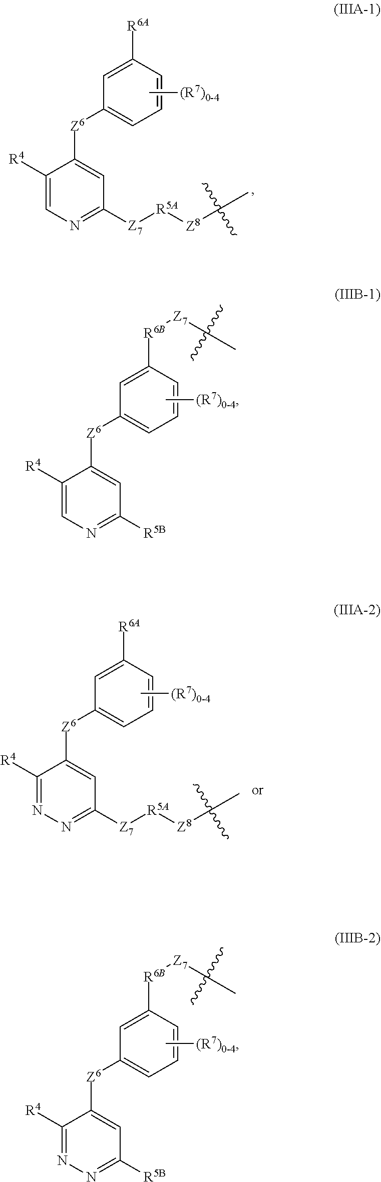

or a pharmaceutically acceptable salt thereof, wherein: R⁴ is R⁴ is hydrogen, —F, —Cl, —Br, —I, —OH, —O—(C₁-C₆ aliphatic), —NH₂, —NH—(C₁-C₆ aliphatic), —N(C₁-C₆ aliphatic)₂, —CN, —C(O)—C₁-C₆ aliphatic, —CO₂H, —C(O)—NH₂, —C(O)—NH(C₁-C₆ aliphatic), —C(O)—N(C₁-C₆ aliphatic)₂, —C(O)—(C₁-C₆ aliphatic), —CD₃, —CF₃, or —O—CF₃; R^{6A} is Ring 6A; Ring 6A is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, wherein the Ring 6A is optionally substituted with one or more R⁸; R^{6B} is Ring 6B; Ring 6B is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, wherein the Ring 6B is optionally substituted with one or more R; each R⁷ is independently hydrogen, —F, —Cl, —Br, —I, —OH, or —O—(C₁-C₆ aliphatic); each R⁸ is independently hydrogen, —F, —Cl, —Br, —I, or —OH; Z⁶ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z⁷ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; Z⁸ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—; each R^{C} is independently hydrogen or an optionally substituted C₁-C₆ aliphatic group,
and

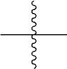

represents the point of attachment to L.

In some embodiments, TBM has a structure of Formula (IIIA-1) or Formula (IIIB-1):

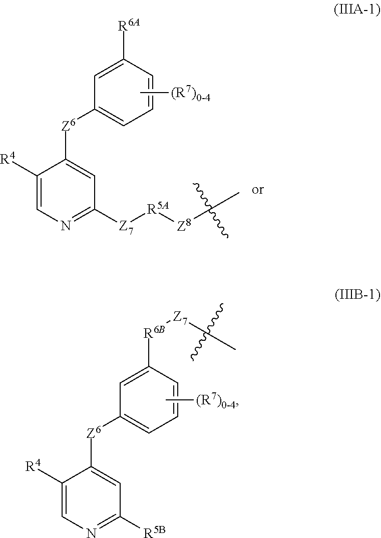

or a pharmaceutically acceptable salt thereof.

In some embodiments, TBM has a structure of Formula (IIIA-2) or Formula (IIIB-2):

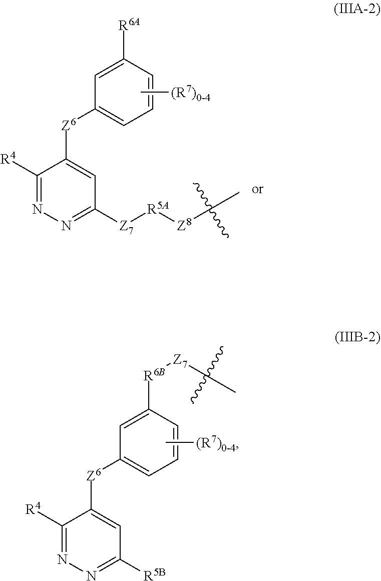

or a pharmaceutically acceptable salt thereof.

In some embodiments, TBM has the structure of Formula (IIIA), wherein R⁴ is hydrogen, —F, —Cl, —Br, —I, —OH, —O—(C₁-C₆ aliphatic), —NH₂, —NH—(C₁-C₆ aliphatic), —N(C₁-C₆ aliphatic)₂, —CN, —C(O)—C₁-C₆ aliphatic, —CO₂H, —C(O)—NH₂, —C(O)—NH(C₁-C₆ aliphatic), —C(O)—N(C₁-C₆ aliphatic)₂, —C(O)—(C₁-C₆ aliphatic), —CD₃, —CF₃, or —O—CF₃.

In some embodiments, TBM has the structure of Formula (IIIA), wherein R⁴ is hydrogen or —C(O)—NH(C₁-C₆ aliphatic).

In some embodiments, TBM has the structure of Formula (IIIA), wherein R⁴ is —C(O)—NH(CH₃).

In some embodiments, TBM has the structure of Formula (IIIA), wherein R^{6A} is Ring 6A.

In some embodiments, Ring 6A is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S.

In some embodiments, TBM has the structure of Formula (IIIB), wherein R^{6B} is Ring 6B.

In some embodiments, Ring 6B is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S.

In some embodiments, each R⁸ is independently hydrogen, —F, —Cl, —Br, —I, or —OH.

In some embodiments, at least one R⁸ is —F.

In some embodiments, each R⁷ is independently hydrogen, —F, —Cl, —Br, —I, —OH, or —O—(C₁-C₆ aliphatic).

In some embodiments, one R⁷ is —O—(C₁-C₆ aliphatic).

In some embodiments, Z⁶ is —NH—.

In some embodiments, TBM has a structure of Formula (IIIA-3-1) or Formula (IIIA-3-2):

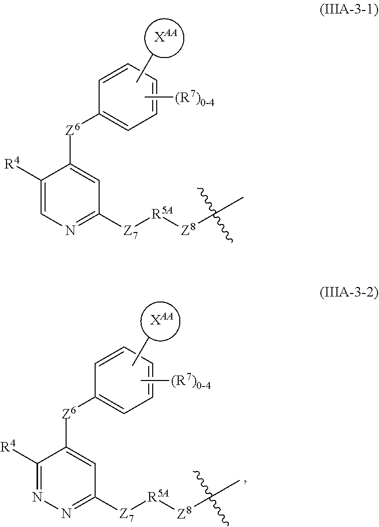

or a pharmaceutically acceptable salt thereof, wherein; Ring X^{AA} is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, or a 5-6 membered heteroaryl having 1-2 heteroatoms independently selected from N, O, and S.

In some embodiments, TBM has a structure of Formula (IIIA-3-1-1) or Formula (IIIA-3-2-1):

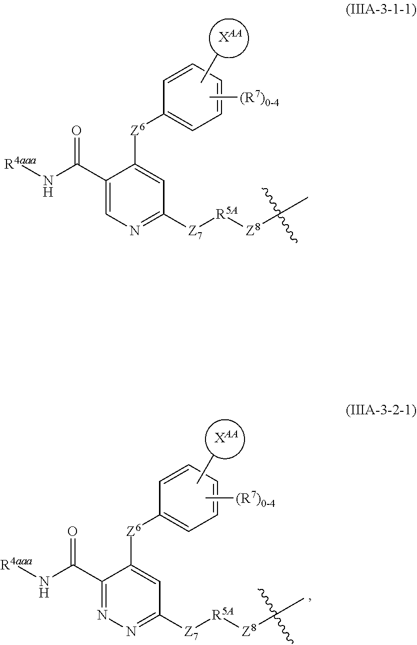

or a pharmaceutically acceptable salt thereof, wherein; R^{4aa} is —CH₃, CD₃, —CH₂CH₃, or

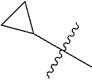 [SMILES: *C1CC1]

In some embodiments, TBM has a structure of Formula (IIIA-3-1-2) or Formula (IIIA-3-2-2):

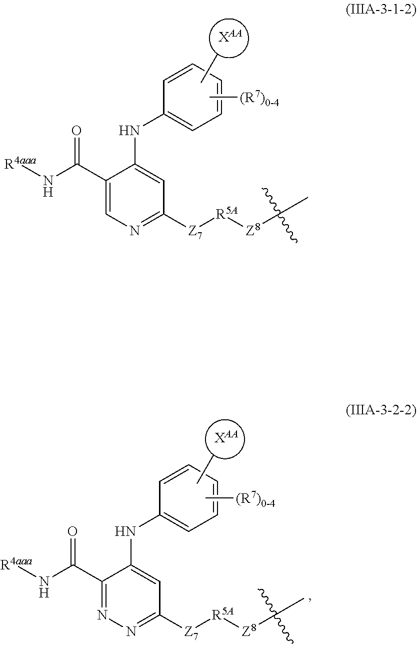

or a pharmaceutically acceptable salt thereof.

In some embodiments, TBM has a structure of Formula (IIIA-3):

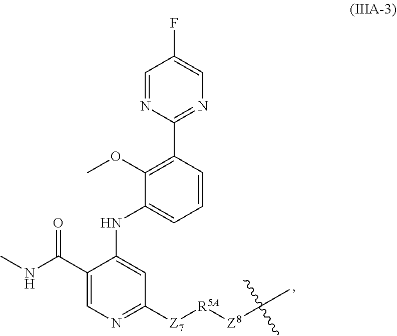

or a pharmaceutically acceptable salt thereof.

In some embodiments, Z⁷ is selected from a covalent bond, —NR^{C}—, —C(O)—, —NR^{C}C(O)—, —C(O)NR^{C}—, and —NR^{C}C(O)NR^{C}—

In some embodiments, Z⁸ is selected from a covalent bond, —C(R^{C}₂)—, —NR^{C}—, —C(O)—, and —C(O)NR^{C}—.

In some embodiments, Z⁸ is —NH—.

In some embodiments, Z⁸ is selected from —C(O)NH— and —C(O)NCH₃—.

In some embodiments, the structure of Formula (IIIA), wherein R^{5A} is a covalent bond.

In some embodiments, the TBM has the structure of Formula (IIIA), wherein R^{5A} is Ring 5A.

In some embodiments, the TBM has the structure of Formula (IIIA), wherein R^{5A} is a 5-6 membered heteroarylenyl having 1-4 heteroatoms independently selected from N, O, and S.

In some embodiments, the TBM has structure of Formula (IIIA), wherein R^{5A} is

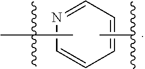

In some embodiments TBM has a structure of Formula (IIIA-3-3)

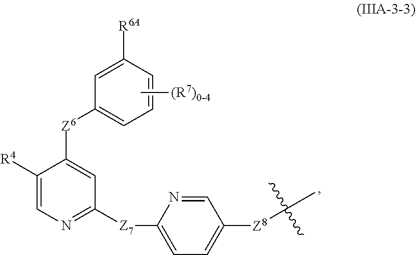

or a pharmaceutically acceptable salt thereof.

In some embodiments, R^{6A} is hydrogen or an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, or a 5-6 membered heteroaryl having 1-2 heteroatoms independently selected from N, O, and S.

In some embodiments, R⁷ is OCH₃.

In some embodiments, Z⁶ is —NH—.

In some embodiments, Z⁷ is —NH—.

In some embodiments, TBM is:

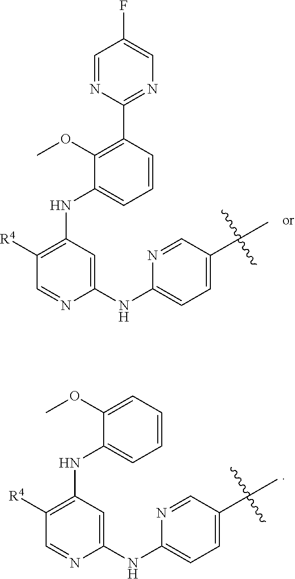

In some embodiments, R⁴ is —C(O)—NH—CH₃, —C(O)—NH—CD₃, —C(O)—NH—CH₂CH₃, or

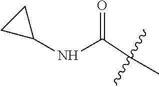 [SMILES: *C(=O)NC1CC1]

In some embodiments, TBM is:

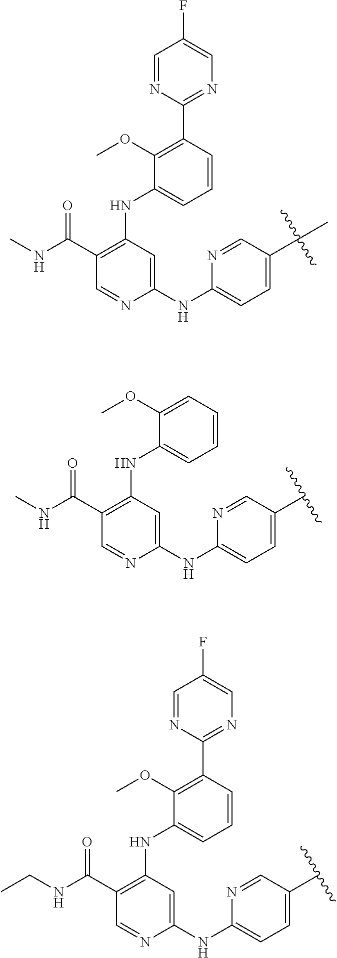 [SMILES: *c1ccc(Nc2cc(Nc3cccc(-c4ncc(F)cn4)c3OC)c(C(=O)NC)cn2)nc1 ; *c1ccc(Nc2cc(Nc3ccccc3OC)c(C(=O)NC)cn2)nc1 ; *c1ccc(Nc2cc(Nc3cccc(-c4ncc(F)cn4)c3OC)c(C(=O)NCC)cn2)nc1]

In some embodiments, the compound of Formula (I-b) has a structure of Formula (I-b-1) or Formula (I-b-2):

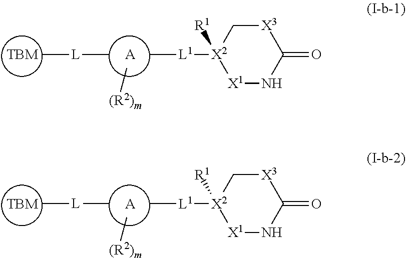

or a pharmaceutically acceptable salt thereof, wherein: each of TBM, Ring A, L, L, R¹, R², X¹, X², X³, and m is as defined above.

In some embodiments, the compound of Formula (I-b) has a structure of Formula (I-b-3):

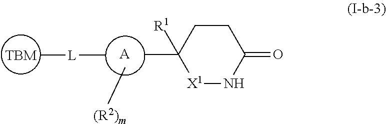

or a pharmaceutically acceptable salt thereof, wherein: each of TBM, Ring A, L, R¹, R², X¹, and m is as defined above.

In some embodiments, the compound has an E3 ubiquitin ligase binding moiety as a cereblon E3 ubiquitin ligase binding moiety, and the compound has the structure of Formula (I-b-4):

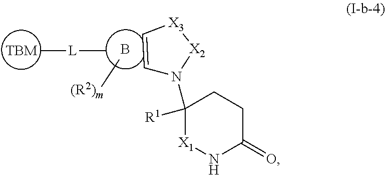

or a pharmaceutically acceptable salt thereof, wherein:
each of X₁, X₂, and X₃ is independently a covalent bond, —CH₂—, —C(O)—, —C(S)—, —NR— or

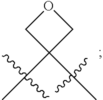 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic group; each of R² is independently at each occurrence hydrogen, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or
—N(R)S(O)₂R; Ring B is a fused ring selected from a 6-membered aryl containing 0-2 nitrogen atoms, a 5 to 7-membered partially saturated carbocyclyl, a 5 to 7-membered partially saturated heterocyclyl having 1-2 heteroatoms independently selected from nitrogen, oxygen or sulfur, or a 5-membered heteroaryl having 1-3 heteroatoms independently selected from nitrogen, oxygen or sulfur; m is an integer from 0 to 4; each R is independently at each occurrence hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, and combinations thereof, or: two R groups on the same nitrogen are optionally taken together with their intervening atoms to form a 4 to 7-membered saturated, partially unsaturated, or heteroaryl ring having 0-3 heteroatoms in addition to the nitrogen, independently selected from N, O, and S.

In some embodiments, X₁ and X₂ are —C(O)— and X₃ is —NR—, wherein R is hydrogen, or an optionally substituted C₁-C₆ aliphatic group.

In some embodiments, R¹ and R² are a hydrogen at each occurrence.

In some embodiments, Ring A is a fused phenyl ring.

In some embodiments, the compound has structure selected from Formula (1-a-11), Formula (1-a′-11), and Formula (1-a″-11):

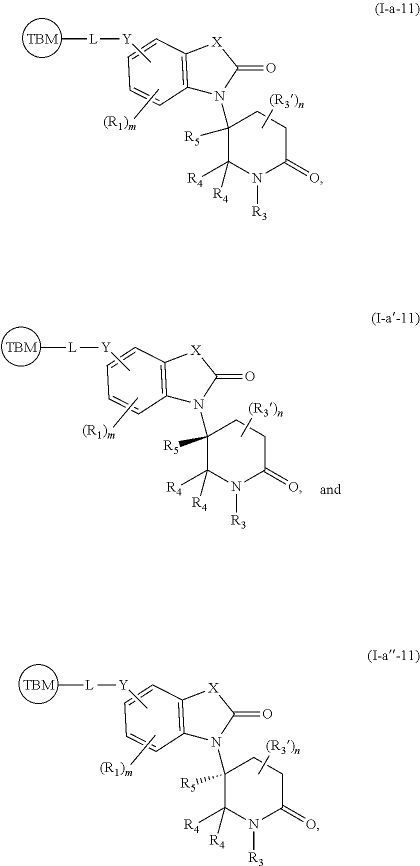

or a pharmaceutically acceptable salt thereof, wherein L and TBM are as defined above, and wherein: Y is a bond, Y₁, O, NH, NR₂, C(O)O, OC(O), C(O)NR₂′, NR₂′C(O), Y₁—O, Y₁—NH, Y₁—NR₂, Y₁—C(O), Y₁—C(O)O, Y₁—OC(O), Y₁—C(O)NR₂′, or Y₁—NR₂′C(O), wherein Y₁ is C₁-C₆ alkylene, C₂-C₆ alkenylene, or C₂-C₆ alkynylene; X is C(O) or C(R₃)₂; each R₁ is independently halogen, nitro, NH₂, OH, C(O)OH, C₁-C₆ alkyl, or C₁-C₆ alkoxy; R₂ is C₁-C₆ alkyl, C₂-C₆ alkenyl, C₃-C₈ cycloalkyl, 3- to 8-membered heterocycloalkyl, C(O)—C₁-C₆ alkyl, C(O)— C₂-C₆ alkenyl, C(O)—C₃-C₈ cycloalkyl, or C(O)—3- to 8-membered heterocycloalkyl, and R₂ is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy; R₂′ is H, C₁-C₆ alkyl, C₂-C₆ alkenyl, C₃-C₈ cycloalkyl, or 3- to 8-membered heterocycloalkyl, and R₂′, when not being H, is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy; each R₃ is independently H or C₁-C₃ alkyl optionally substituted with C₆-C₁₀ aryl or 5- to 10-membered heteroaryl; each R₃′ is independently C₁-C₃ alkyl; each R₄ is independently H or C₁-C₃ alkyl; or two R₄, together with the carbon atom to which they are attached, form C(O), a C₃-C₆ carbocycle, or a 4-, 5-, or 6-membered heterocycle comprising 1 or 2 heteroatoms selected from N and O; R₅ is H, C₁-C₃ alkyl, F, or Cl; each Rₐ independently is H or C₁-C₆ alkyl; R_{b} is H or tosyl; t is 0 or 1; m is 0, 1, 2 or 3; and n is 0, 1 or 2.

In some embodiments, the E3 ubiquitin ligase binding moiety is

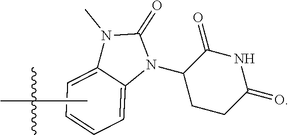

In some embodiments, E3 ubiquitin ligase binding moiety is

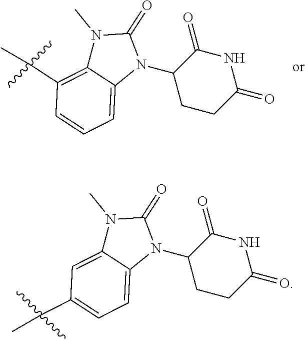 [SMILES: *c1cccc2c1n(C)c(=O)n2C1CCC(=O)NC1=O ; *c1ccc2c(c1)n(C)c(=O)n2C1CCC(=O)NC1=O]

In some embodiments, the compound has a structure of:

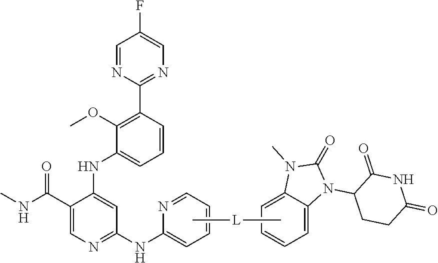

or a pharmaceutically acceptable salt thereof.

In some embodiments, the structure is selected from the group consisting of:

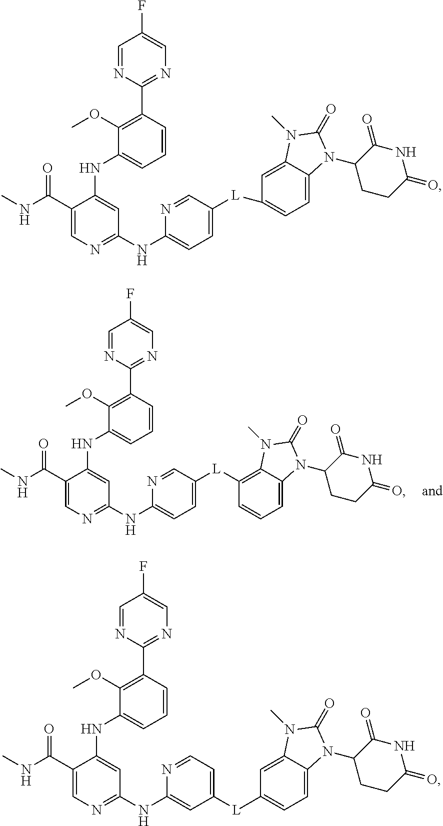

or a pharmaceutically acceptable salt thereof.

In some embodiments, L is a covalent bond or a bivalent, saturated or partially unsaturated, straight or branched C₁-C₂₀ alkylene chain, wherein 0-6 methylene units of L are independently replaced by -Cy-, —O—, —NR^{C}—, —S—, —OC(O)—, —C(O)O—, —C(O)—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—, and combinations thereof, wherein -Cy- is independently at each occurrence an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl, having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur, and each R^{C} is independently at each occurrence hydrogen, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, and combinations thereof.

In some embodiments, L is a covalent bond.

In some embodiments, L comprises a saturated straight C₁-C₁₂ alkylene chain.

In some embodiments, L comprises a saturated straight C₁-C₈ alkylene chain.

In some embodiments, L comprises a saturated straight C₂-C₆ alkylene chain.

In some embodiments, L comprises a saturated straight C₄ alkylene chain.

In some embodiments, 0-5 methylene units of L are independently replaced by -Cy-, —O—, —NR^{C}—, —N(R^{C})C(O)—, —C(O)N(R^{C})—, —OC(O)N(R^{C})—, and —N(R^{C})C(O)O—, and combinations thereof,

In some embodiments, L comprises a polyethylene glycol (PEG) (

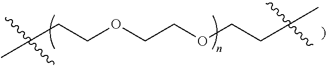

chain, wherein n is an integer from 1 to 10.

In some embodiments, L comprises at least one -Cy-.

In some embodiments, -Cy- is each independently an optionally substituted bivalent ring selected from phenylenyl, a 4-6 membered saturated or partially unsaturated carbocyclylenyl, a 6-10 membered saturated or partially unsaturated spiro carbocyclylenyl, a 4-7 membered saturated or partially unsaturated heterocyclylenyl having 1-2 heteroatoms independently selected from nitrogen, oxygen, and sulfur, a 8-10 membered saturated or partially unsaturated spiro heterocyclylenyl, having 1-2 heteroatoms independently selected from nitrogen, oxygen, and sulfur, or a 5-6 membered heteroarylenyl having 1-4 heteroatoms independently selected from nitrogen, oxygen, and sulfur.

In some embodiments, -Cy- is each independently an optionally substituted bivalent ring selected from phenylenyl, a 4-6 membered saturated or partially unsaturated carbocyclylenyl, a 6-10 membered saturated or partially unsaturated spiro carbocyclylenyl, a 4-7 membered saturated or partially unsaturated heterocyclylenyl having 1-2 nitrogen atoms, a 8-10 membered saturated or partially unsaturated spiro heterocyclylenyl, having 1-2 nitrogen atoms, or a 5-6 membered heteroarylenyl having 1-3 nitrogen atoms.

In some embodiments, -Cy- is each independently an optionally substituted bivalent ring selected from phenylenyl, a 4 or 6 membered saturated or partially unsaturated carbocyclylenyl, a 9 membered saturated or partially unsaturated spiro carbocyclylenyl, a 4 or 6 membered saturated or partially unsaturated heterocyclylenyl having 1-2 nitrogen atoms, a 4 or 6 membered saturated or partially unsaturated spiro heterocyclylenyl, having 1-2 nitrogen atoms, or a 5 or 6 membered heteroarylenyl having 1-3 nitrogen atoms.

In some embodiments, L comprises a structure selected from the group consisting of:

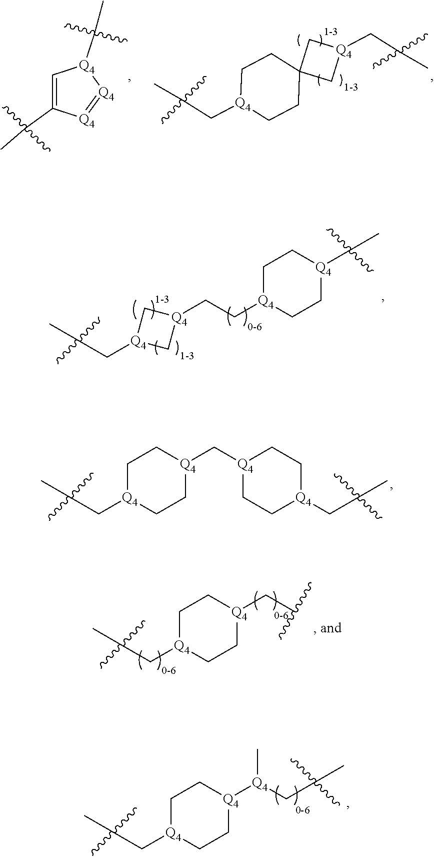

where each Q₄ is independently selected from —CH— and —N— when Q₄ is attached to only single bonds, or Q₄ is ═CH— or ═N— when Q₄ is attached to a double bond.

In some embodiments, L comprises a structure selected from the group consisting of:

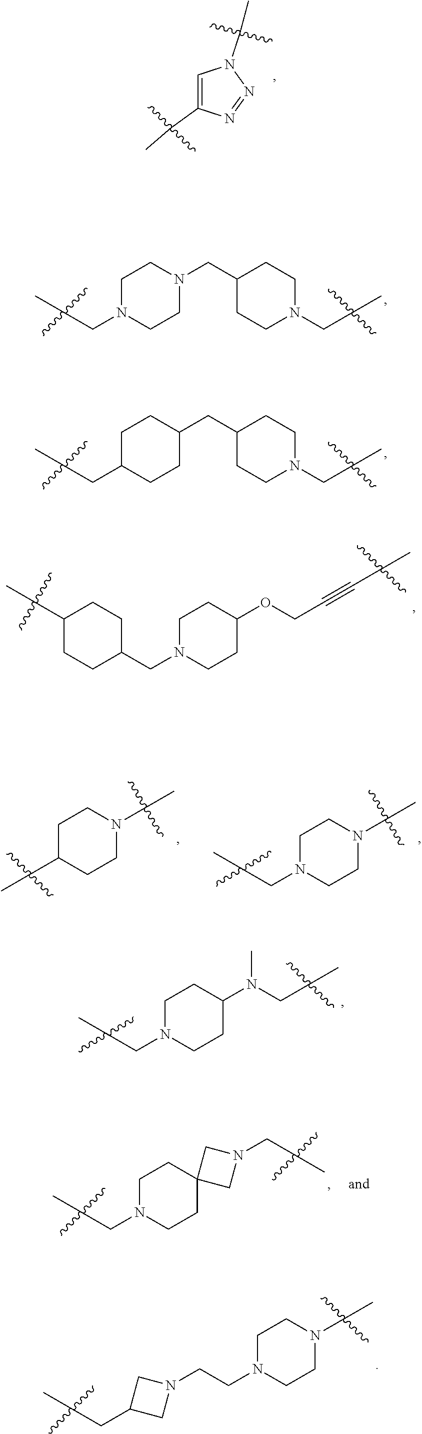

In some embodiments, L comprises a structure selected from the group consisting of:

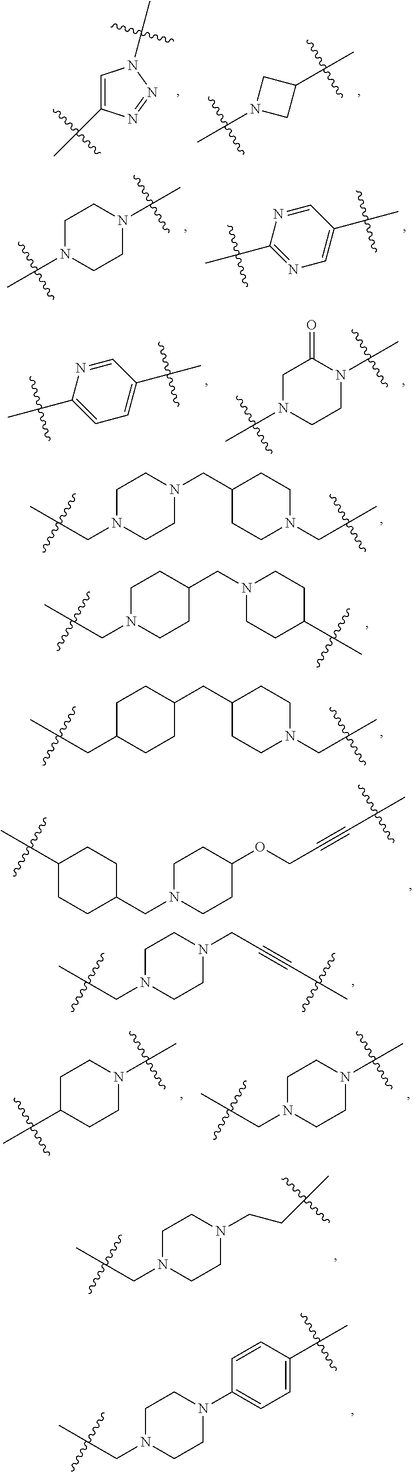

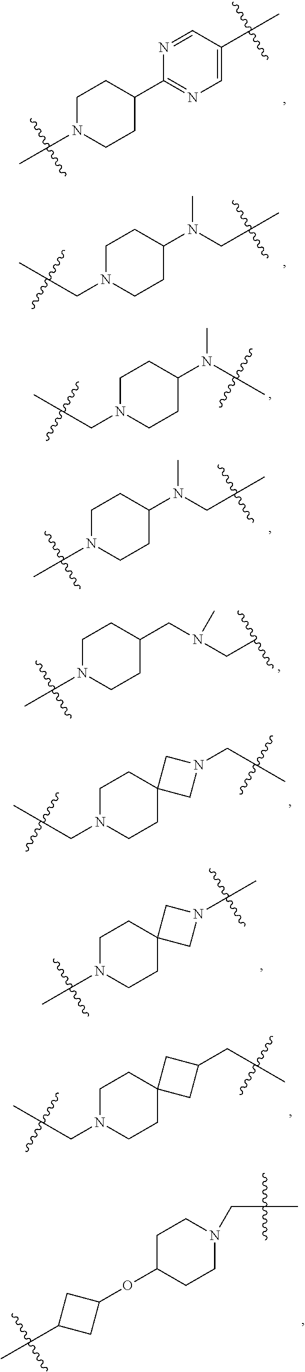

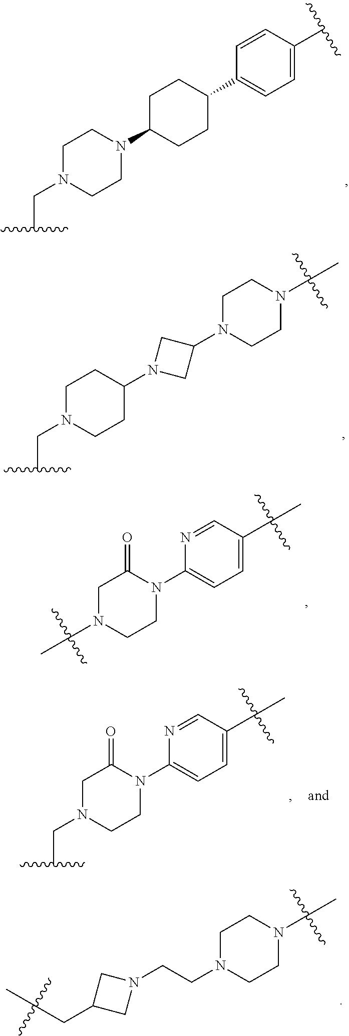 [SMILES: *CN1CCN([C@H]2CC[C@H](c3ccc(*)cc3)CC2)CC1 ; *CN1CCC(N2CC(N3CCN(*)CC3)C2)CC1 ; *c1ccc(N2CCN(*)CC2=O)nc1 ; *CN1CCN(c2ccc(*)cn2)C(=O)C1 ; *CC1CN(CCN2CCN(*)CC2)C1]

In some embodiments, L comprises a structure selected from the group consisting of:

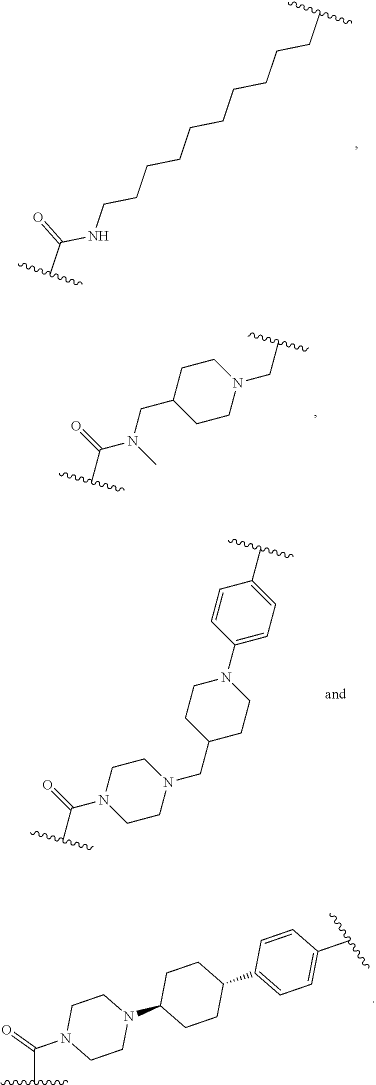 [SMILES: *CCCCCCCCCCNC(*)=O ; *CN1CCC(CN(C)C(*)=O)CC1 ; *C(=O)N1CCN(CC2CCN(c3ccc(*)cc3)CC2)CC1 ; *C(=O)N1CCN([C@H]2CC[C@H](c3ccc(*)cc3)CC2)CC1]

In some embodiments, L comprises a structure selected from the group consisting of:

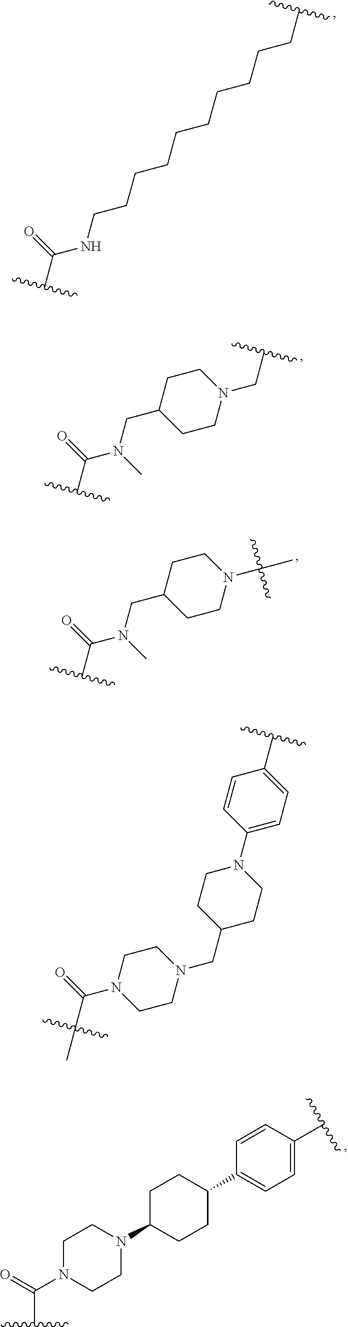 [SMILES: *CCCCCCCCCCNC(*)=O ; *CN1CCC(CN(C)C(*)=O)CC1 ; *C(=O)N(C)CC1CCN(*)CC1 ; *C(=O)N1CCN(CC2CCN(c3ccc(*)cc3)CC2)CC1 ; *C(=O)N1CCN([C@H]2CC[C@H](c3ccc(*)cc3)CC2)CC1]

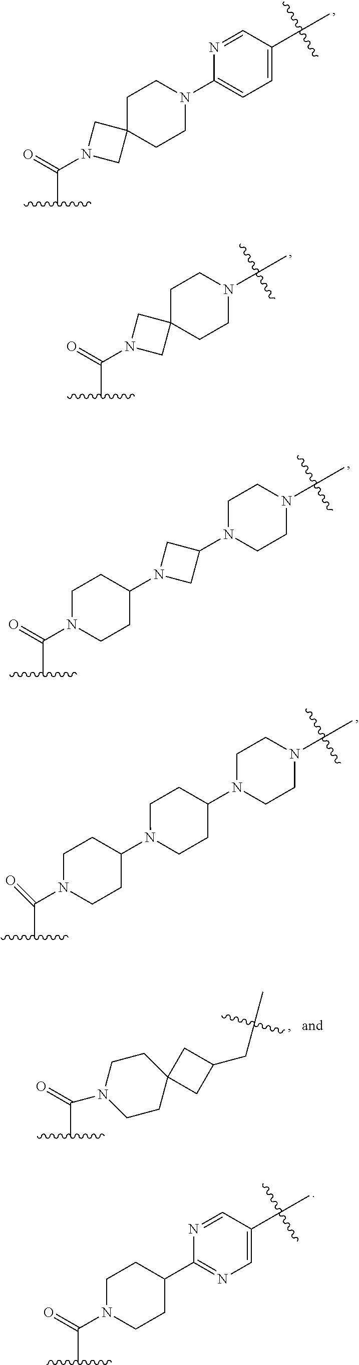 [SMILES: *C(=O)N1CC2(CCN(c3ccc(*)cn3)CC2)C1 ; *C(=O)N1CC2(CCN(*)CC2)C1 ; *C(=O)N1CCC(N2CC(N3CCN(*)CC3)C2)CC1 ; *C(=O)N1CCC(N2CCC(N3CCN(*)CC3)CC2)CC1 ; *CC1CC2(CCN(C(*)=O)CC2)C1 ; *C(=O)N1CCC(c2ncc(*)cn2)CC1]

In some embodiments, L comprises a structure selected from the group consisting of:

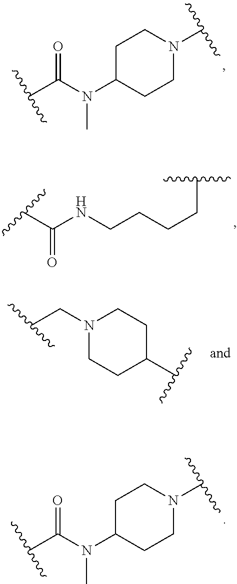 [SMILES: *C(=O)N(C)C1CCN(*)CC1 ; *CCCCNC(*)=O ; *CN1CCC(*)CC1 ; *C(=O)N(C)C1CCN(*)CC1]

In some embodiments, L comprises a structure selected from the group consisting of:

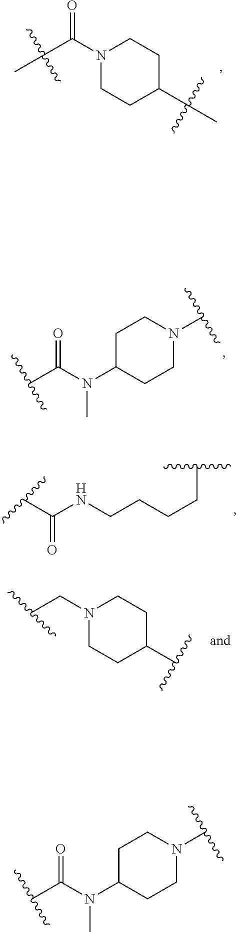 [SMILES: *C(=O)N1CCC(*)CC1 ; *C(=O)N(C)C1CCN(*)CC1 ; *CCCCNC(*)=O ; *CN1CCC(*)CC1 ; *C(=O)N(C)C1CCN(*)CC1]

In some embodiments,

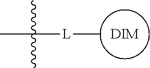

is selected from the group consisting of

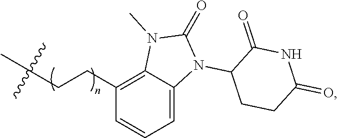

wherein n is an integer from 1 to 8.

In another aspect, provided herein is a compound is represented by Formula (II-ABC′):

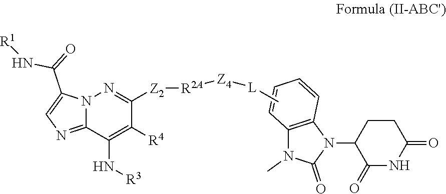

or a pharmaceutically acceptable salt thereof, wherein:
R¹ is H, —C(CH₃)₂—CN,

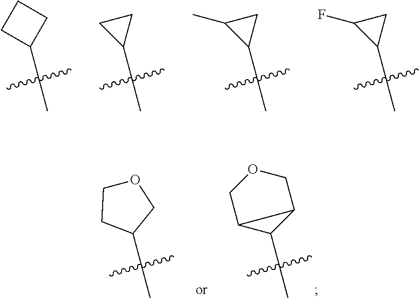

R³ is H or C₁-C₆ aliphatic; R⁴ is H; or R³ and R⁴ are joined together to form a 5 or 6-membered heterocyclic ring; Z₂ is a covalent bond or —NH—; Z₄ is a covalent bond, —NH—, or —O—;
R^{2A} is

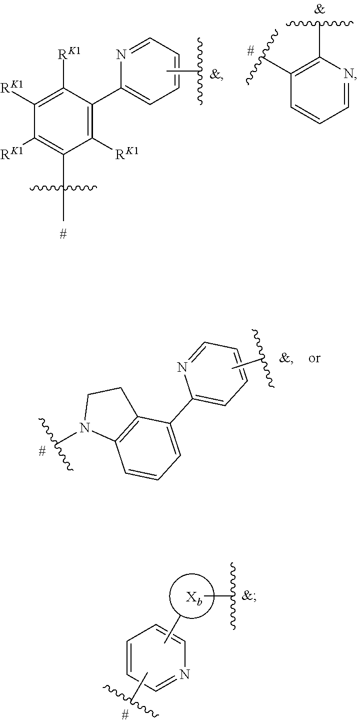

each R^{K1} is independently hydrogen, halo, C₁-C₆ aliphatic, halo substituted C₁-C₆ aliphatic, or —O—(C₁-C₆ aliphatic); Ring X_{b} is a ring selected from the group consisting of phenyl, a 4 to 7-membered saturated or partially unsaturated carbocyclyl or heterocyclyl with 1-3 heteroatoms independently selected from N, O, and S, and a 5 to 6-membered heteroaryl with 1-4 heteroatoms independently selected from N, O, and S, and each of which is optionally substituted with one or more of R^{K}; each R^{K} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, a C₁-C₆ aliphatic group, or two R^{K} groups attached to the same carbon or sulfur atom are optionally taken together to form ═O; n is 1-4; wherein each #indicates the point of attachment to Z₂ and each & indicates the point of attachment to Z₄; and L is a covalent bond or a bivalent, saturated or partially unsaturated, straight or branched C₁-C₅ alkylene chain, wherein 0-6 methylene units of L are independently replaced by -Cy-, —O—, —NR^{C}—, —S—, —OC(O)—, —C(O)O—, —C(O)—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—, C₂-C₃ alkynylene and combinations thereof, wherein -Cy- is independently at each occurrence an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl, having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur, and each R^{C} is independently at each occurrence hydrogen, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, and combinations thereof.

In some embodiments, the compound is represented by Formula (II-ABC):

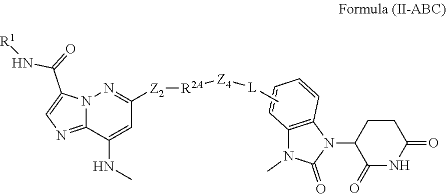

or a pharmaceutically acceptable salt thereof,
wherein: R¹ is H, —C(CH₃)₂—CN,

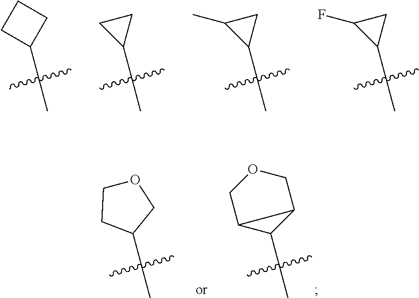

Z₂ is a covalent bond or —NH—; Z₄ is a covalent bond, —NH—, or —O—;
R^{2A} is

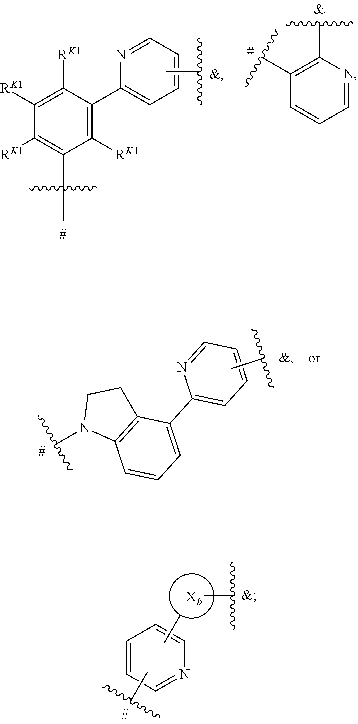

each R^{K1} is independently hydrogen, halo, C₁-C₆ aliphatic, halo substituted C₁-C₆ aliphatic, or —O—(C₁-C₆ aliphatic); Ring X_{b} is a ring selected from the group consisting of phenyl, a 4 to 7-membered saturated or partially unsaturated carbocyclyl or heterocyclyl with 1-3 heteroatoms independently selected from N, O, and S, and a 5 to 6-membered heteroaryl with 1-4 heteroatoms independently selected from N, O, and S, and each of which is optionally substituted with one or more of R^{K}; each R^{K} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, a C₁-C₆ aliphatic group, or two R^{K} groups attached to the same carbon or sulfur atom are optionally taken together to form ═O; n is 1-4; wherein each #indicates the point of attachment to Z₂ and each & indicates the point of attachment to Z₄; and L is a covalent bond or a bivalent, saturated or partially unsaturated, straight or branched C₁-C₅ alkylene chain, wherein 0-6 methylene units of L are independently replaced by -Cy-, —O—, —NR^{C}—, —S—, —OC(O)—, —C(O)O—, —C(O)—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—, C₂-C₃ alkynylene and combinations thereof, wherein -Cy- is independently at each occurrence an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl, having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur, and each R^{C} is independently at each occurrence hydrogen, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, and combinations thereof.

In some embodiments, L is bivalent, saturated or partially unsaturated, straight or branched C₁-C₈ alkylene chain, wherein 0-6 methylene units of L are independently replaced by -Cy-, —O—, —NR^{C}—, —C(O)—, C₂-C₃ alkynylene, and combinations thereof and combinations thereof.

In some embodiments, R¹ is

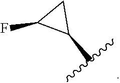 [SMILES: *[C@@H]1C[C@@H]1F]

In some embodiments, the compound is represented by Formula (II-ABC-I):

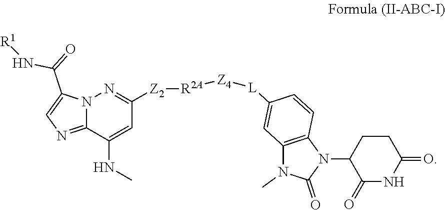

In some embodiments, the compound is represented by Formula (II-ABC-II):

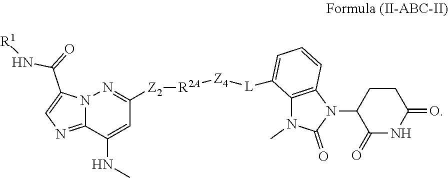

In some embodiments, L comprises a structure selected from the group consisting of:

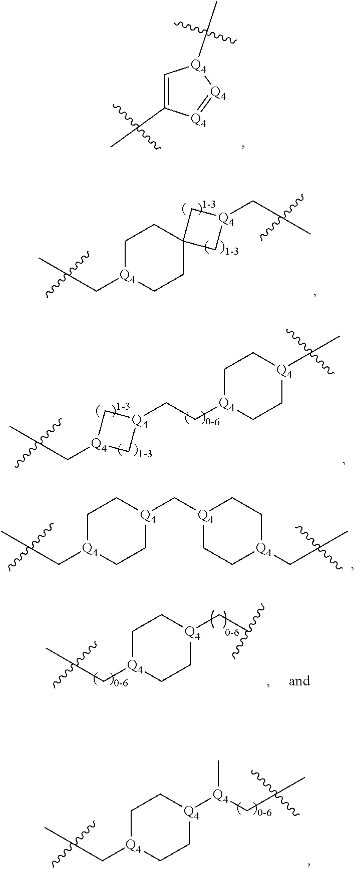

where each Q₄ is independently selected from —CH— and —N— when Q₄ is attached to only single bonds, or Q₄ is ═CH— or ═N— when Q₄ is attached to a double bond.

In some embodiments, L comprises a structure selected from the group consisting of:

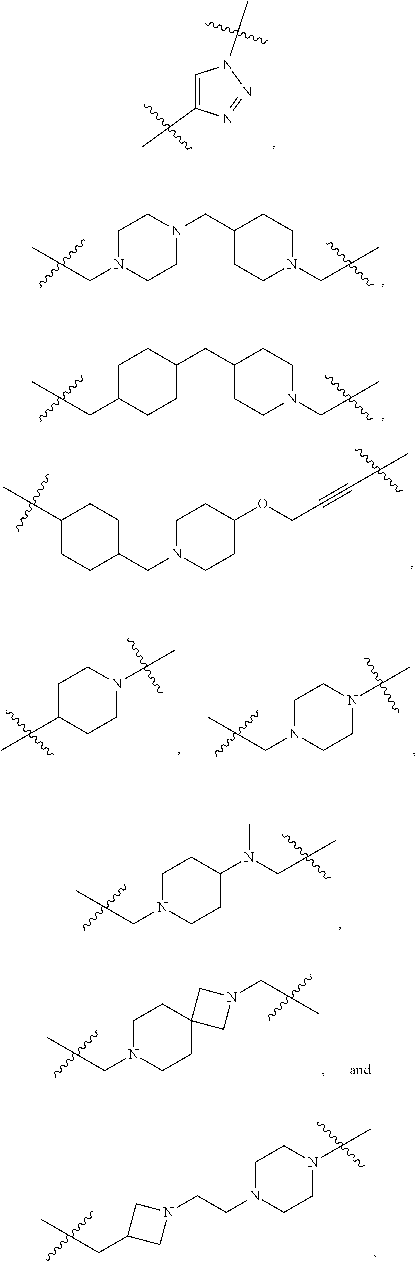

In some embodiments, L comprises a structure selected from the group consisting of:

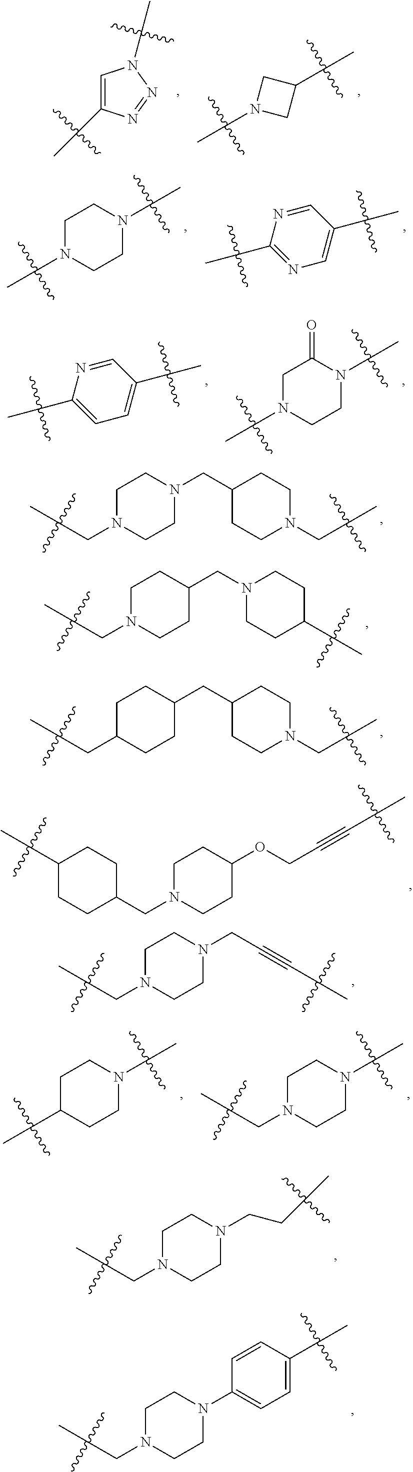

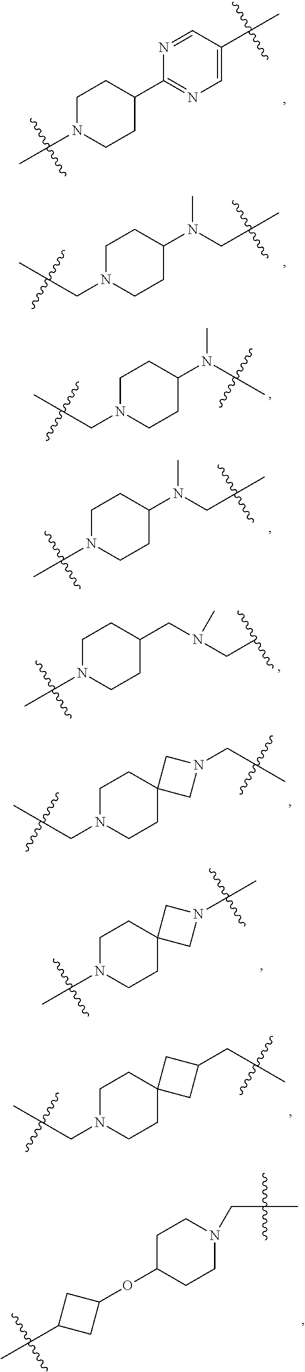

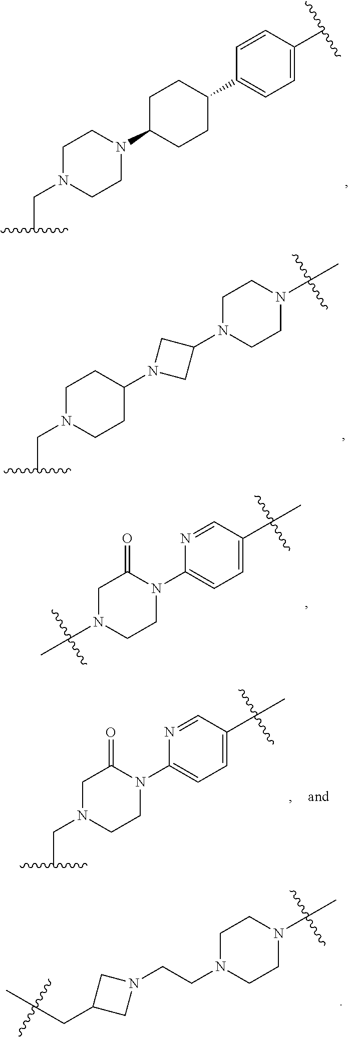 [SMILES: *CN1CCN([C@H]2CC[C@H](c3ccc(*)cc3)CC2)CC1 ; *CN1CCC(N2CC(N3CCN(*)CC3)C2)CC1 ; *c1ccc(N2CCN(*)CC2=O)nc1 ; *CN1CCN(c2ccc(*)cn2)C(=O)C1 ; *CC1CN(CCN2CCN(*)CC2)C1]

In some embodiments, L comprises a structure selected from the group consisting of:

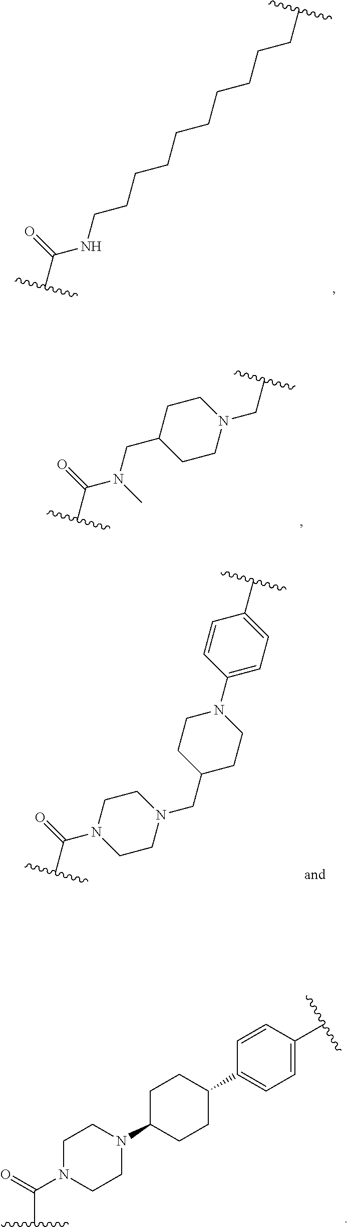 [SMILES: *CCCCCCCCCCNC(*)=O ; *CN1CCC(CN(C)C(*)=O)CC1 ; *C(=O)N1CCN(CC2CCN(c3ccc(*)cc3)CC2)CC1 ; *C(=O)N1CCN([C@H]2CC[C@H](c3ccc(*)cc3)CC2)CC1]

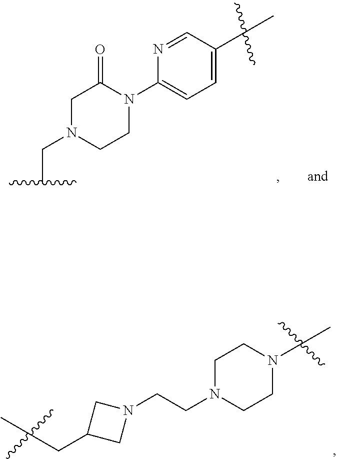 [SMILES: *CN1CCN(c2ccc(*)cn2)C(=O)C1 ; *CC1CN(CCN2CCN(*)CC2)C1]

In some embodiments, L comprises a structure selected from the group consisting of:

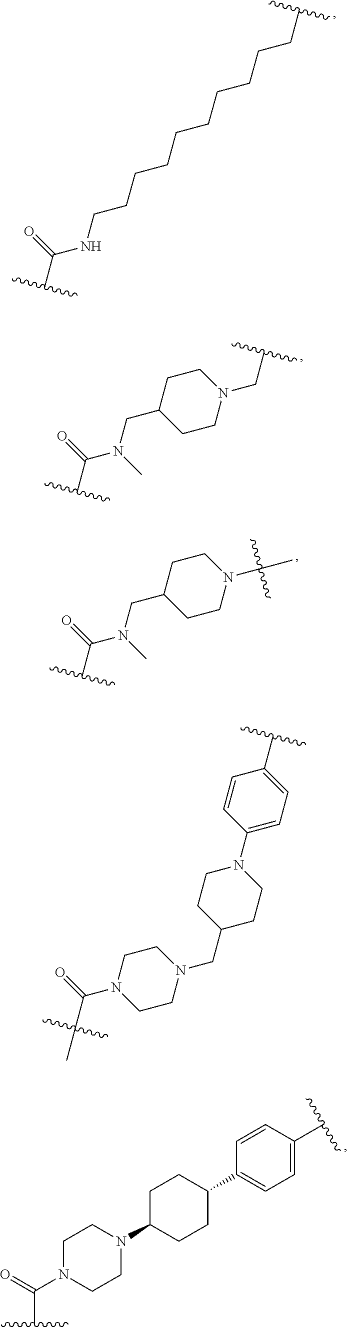 [SMILES: *CCCCCCCCCCNC(*)=O ; *CN1CCC(CN(C)C(*)=O)CC1 ; *C(=O)N(C)CC1CCN(*)CC1 ; *C(=O)N1CCN(CC2CCN(c3ccc(*)cc3)CC2)CC1 ; *C(=O)N1CCN([C@H]2CC[C@H](c3ccc(*)cc3)CC2)CC1]

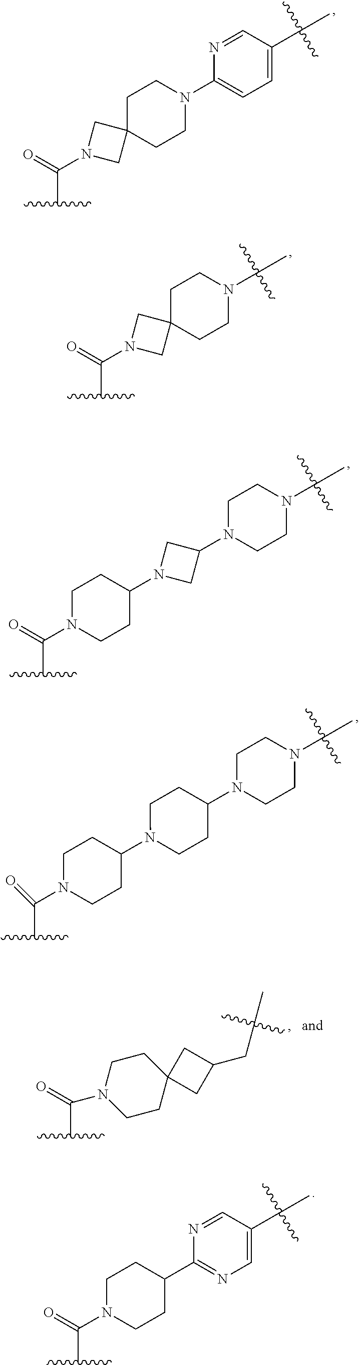 [SMILES: *C(=O)N1CC2(CCN(c3ccc(*)cn3)CC2)C1 ; *C(=O)N1CC2(CCN(*)CC2)C1 ; *C(=O)N1CCC(N2CC(N3CCN(*)CC3)C2)CC1 ; *C(=O)N1CCC(N2CCC(N3CCN(*)CC3)CC2)CC1 ; *CC1CC2(CCN(C(*)=O)CC2)C1 ; *C(=O)N1CCC(c2ncc(*)cn2)CC1]

In some embodiments, L comprises a structure selected from the group consisting of:

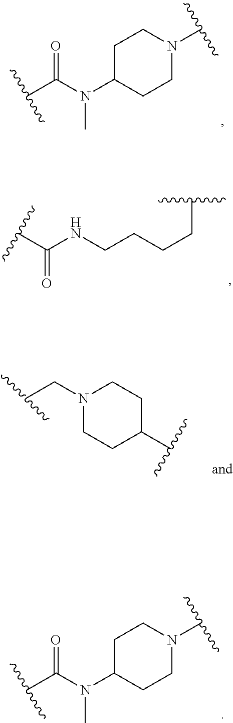 [SMILES: *C(=O)N(C)C1CCN(*)CC1 ; *CCCCNC(*)=O ; *CN1CCC(*)CC1 ; *C(=O)N(C)C1CCN(*)CC1]

In some embodiments, L comprises a structure selected from the group consisting of:

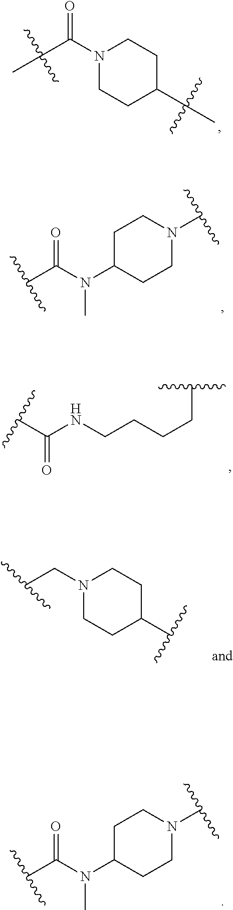 [SMILES: *C(=O)N1CCC(*)CC1 ; *C(=O)N(C)C1CCN(*)CC1 ; *CCCCNC(*)=O ; *CN1CCC(*)CC1 ; *C(=O)N(C)C1CCN(*)CC1]

In some embodiments, the compound is selected from any one of the compounds depicted in Table 1, or a pharmaceutically acceptable salt thereof.

In some embodiments, a compound described herein is represented by Formula (IV):

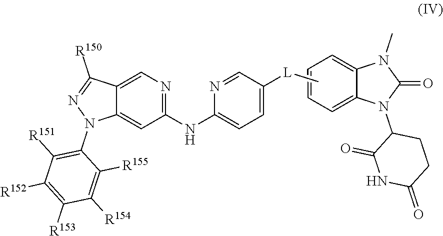

wherein: R¹⁵⁰ is C₁-C₃ aliphatic; and each of R¹⁵¹, R¹⁵², R¹⁵³, R¹⁵⁴, and R¹⁵⁵ is independently hydrogen, halogen, or —O—(C₁-C₆ aliphatic). In some embodiments, R¹⁵⁰ is —CH₂CH₃ or —C₂CH₃. In some embodiments, each of R¹⁵¹ and R¹⁵² is independently halogen, or —O—(C₁-C₆ aliphatic). In some embodiments, each of R¹⁵¹ and R¹⁵² is independently fluoro or methoxy. In some embodiments, each of R¹⁵³, R¹⁵⁴, and R¹⁵⁵ is hydrogen.

In some embodiments, the compound is of Formula (IV-A):

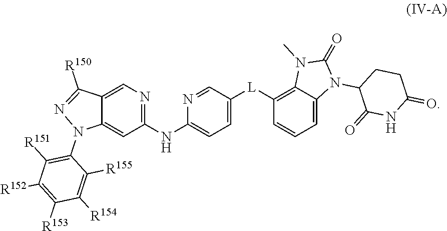

In some embodiments, TBM as described herein comprises (e.g., consists of) Formula (IV-I):

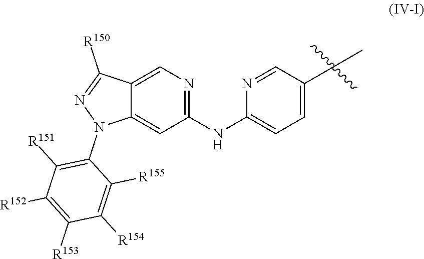

wherein: R¹⁵⁰ is C₁-C₃ aliphatic; and each of R¹⁵¹, R¹⁵², R¹⁵³, R¹⁵⁴, and R¹⁵⁵ is independently hydrogen, halogen, or —O—(C₁-C₆ aliphatic). In some embodiments, R¹⁵⁰ is —CH₂CH₃ or —C₂CH₃. In some embodiments, each of R¹⁵¹ and R¹⁵² is independently halogen, or —O—(C₁-C₆ aliphatic). In some embodiments, each of R¹⁵¹ and R¹⁵² is independently fluoro or methoxy. In some embodiments, each of R¹⁵³, R¹⁵⁴, and R¹⁵⁵ is hydrogen.

### General Methods of Providing the Present Compounds

The compounds of this invention may be prepared or isolated in general by synthetic and/or semi-synthetic methods known to those skilled in the art for analogous compounds and by methods described in detail in the Examples, herein.

In the Schemes below, where a particular protecting group, leaving group, or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benzylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

In the schemes below, where a provided compound is formed having a reactive moiety (e.g., amine, alcohol, etc.), it is not shown but it is generally appreciated and well known by those having ordinary skill in the art that the reactivity of said reactive moiety may be masked by employing a suitable protecting group that can thereafter be removed in situ or during a separate synthetic step.

In certain embodiments, compounds of the present invention are generally prepared according to Scheme 1 set forth below:

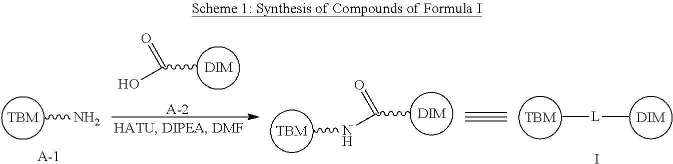

As depicted in Scheme 1, above, amine A-1 is coupled to acid A-2 using the coupling agent HATU in the presence of the base DIPEA in DMF to form a compound of Formula I with a linker comprising an amide bond. The squiggly bond, [symbol] , represents the portion of the linker between TBM and the terminal amino group of A-1 or the portion of the linker between DIM and the terminal carboxyl group of A-2, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 2 set forth below:

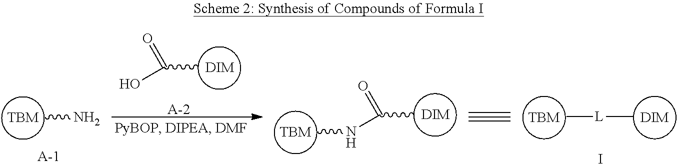

As depicted in Scheme 2, above, amine A-1 is coupled to acid A-2 using the coupling agent PyBOP in the presence of the base DIPEA in DMF to form a compound of Formula I with a linker comprising an amide bond. The squiggly bond, [symbol] , represents the portion of the linker between TBM and the terminal amino group of A-1 or the portion of the linker between DIM and the terminal carboxyl group of A-2, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 3 set forth below:

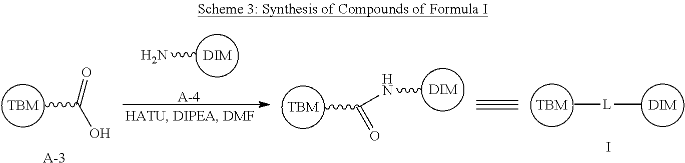

As depicted in Scheme 3, above, acid A-3 is coupled to amine A-4 using the coupling agent HATU in the presence of the base DIPEA in DMF to form a compound of Formula I with a linker comprising an amide bond. The squiggly bond, [symbol] , represents the portion of the linker between TBM and the terminal carboxyl group of A-3 or the portion of the linker between DIM and the terminal amino group of A-4, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 4 set forth below:

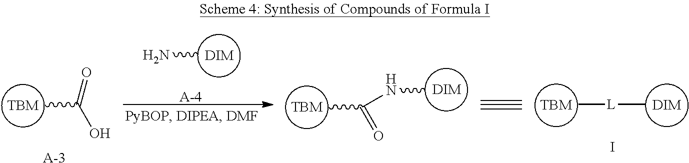

As depicted in Scheme 4, above, acid A-3 is coupled to amine A-4 using the coupling agent PyBOP in the presence of the base DIPEA in DMF to form a compound of Formula I with a linker comprising an amide bond. The squiggly bond, [symbol] , represents the portion of the linker between TBM and the terminal carboxyl group of A-3 or the portion of the linker between DIM and the terminal amino group of A-4, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 5 set forth below:

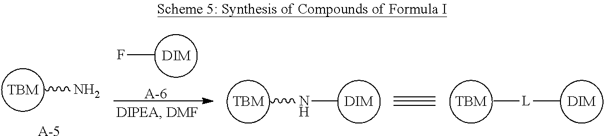

As depicted in Scheme 5, above, an SNAr displacement of fluoride A-6 by amine A-5 is effected in the presence of the base DIPEA in DMF to form a compound of Formula I with a linker comprising a secondary amine. The squiggly bond, [symbol] , represents the portion of the linker between TBM and the terminal amino group of A-5.

In certain embodiments, compounds of the present invention are generally prepared according to Scheme 6 set forth below:

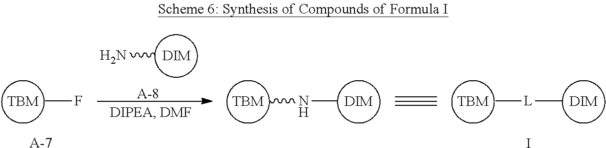

As depicted in Scheme 6, above, an SNAr displacement of fluoride A-7 by amine A-8 is effected in the presence of the base DIPEA in DMF to form a compound of Formula I with a linker comprising a secondary amine. The squiggly bond, [symbol] , represents the portion of the linker between DIM and the terminal amino group of A-8.

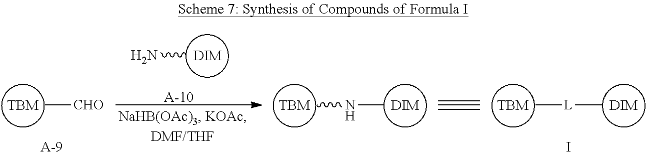

As depicted in Scheme 7, above, reductive amination of the mixture of aldehyde A-9 and amine A-10 is effected in the presence of NaHB(OAc)3 and KOAc in DMF/THF to form a compound of Formula I with a linker comprising a secondary amine. The, [symbol] squiggly bond, represents the portion of the linker between DIM and the terminal amino group of A-8.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March's Advanced Organic Chemistry”, 5^{th} Ed., Ed.: Smith, M. B. and March, J., John Wiley & Sons, New York: 2001, the entirety of which is incorporated herein by reference. Such interconversions may require one or more of the aforementioned techniques, and certain methods for synthesizing compounds of the invention are described below in the Exemplification.

### Uses, Formulation and Administration

### Pharmaceutically Acceptable Compositions

According to another embodiment, the invention provides a composition or a pharmaceutical dosage form comprising a compound of this invention or a pharmaceutically acceptable derivative thereof and a pharmaceutically acceptable carrier, adjuvant, or vehicle. The amount of compound in compositions of this invention is such that is effective to measurably degrade and/or inhibit a TYK2 protein, or a mutant thereof, in a biological sample or in a patient. In certain embodiments, the amount of compound in compositions of this invention is such that is effective to measurably degrade and/or inhibit an TYK2 protein, or a mutant thereof, in a biological sample or in a patient. In certain embodiments, a composition of this invention is formulated for administration to a patient in need of such composition. In some embodiments, a composition of this invention is formulated for oral administration to a patient. In some embodiments, a composition of this invention is for use in the manufacture of a medicament for the treatment of a TYK2 mediated disorder, disease, or condition in a patient.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

Compounds and compositions described herein are generally useful for the inhibition of kinase activity of one or more enzymes. In some embodiments the kinase inhibited by the compounds and methods of the invention is TYK2.

In some embodiments, the invention provides a method of modulating TYK2 in a subject or biological sample comprising administering to the subject, or contacting the biological sample with the compound of Formula (I).

In some embodiments, modulating TYK2 comprises inhibiting or degrading TYK2.

In some embodiments, the invention provides a method of treating a TYK2-mediated disorder, disease, or condition in a subject in need thereof comprising administering to the subject the compound of Formula (I) or its pharmaceutical composition or pharmaceutical dosage form.

TYK2 is a non-receptor tyrosine kinase member of the Janus kinase (JAKs) family of protein kinases. The mammalian JAK family consists of four members, TYK2, JAK1, JAK2, and JAK3. JAK proteins, including TYK2, are integral to cytokine signaling. TYK2 associates with the cytoplasmic domain of type I and type II cytokine receptors, as well as interferon types I and III receptors, and is activated by those receptors upon cytokine binding. Cytokines implicated in TYK2 activation include interferons (e.g. IFN-α, IFN-β, IFN-κ, IFN-δ, IFN-ε, IFN-τ, IFN-ω, and IFN-ζ (also known as limiting), and interleukins (e.g. IL-4, IL-6, IL-10, IL-11, IL-12, IL-13, IL-22, IL-23, IL-27, IL-31, oncostatin M, ciliary neurotrophic factor, cardiotrophin 1, cardiotrophin-like cytokine, and LIF). Velasquez et al., “A protein kinase in the interferon α/β signaling pathway,” Cell (1992) 70:313; Stahl et al., “Association and activation of Jak-Tyk kinases by CNTF-LIF-OSM-IL-60 receptor components,” Science (1994) 263:92; Finbloom et al., “IL-10 induces the tyrosine phosphorylation of Tyk2 and Jak1 and the differential assembly of Stat1 and Stat3 complexes in human T cells and monocytes,” J. Immunol. (1995) 155:1079; Bacon et al., “Interleukin 12 (IL-12) induces tyrosine phosphorylation of Jak2 and Tyk2: differential use of Janus family kinases by IL-2 and IL-12,” J. Exp. Med. (1995) 181:399; Welham et al., “Interleukin-13 signal transduction in lymphohemopoietic cells: similarities and differences in signal transduction with interleukin-4 and insulin,” J. Biol. Chem. (1995) 270:12286; Parham et al., “A receptor for the heterodimeric cytokine IL-23 is composed of IL-12Rβ1 and a novel cytokine receptor subunit, IL-23R,” J. Immunol. (2002) 168:5699. The activated TYK2 then goes on to phosphorylate further signaling proteins such as members of the STAT family, including STAT1, STAT2, STAT4, and STAT6.

TYK2 activation by IL-23, has been linked to inflammatory bowel disease (IBD), Crohn's disease, and ulcerative colitis. Duerr et al., “A Genome-Wide Association Study Identifies IL23R as an Inflammatory Bowel Disease Gene,” Science (2006) 314:1461-1463. As the downstream effector of IL-23, TYK2 also plays a role in psoriasis, ankylosing spondylitis, and Behçet's disease. Cho et al., “Genomics and the multifactorial nature of human auto-immune disease,” N. Engl. J. Med (2011) 365:1612-1623; Cortes et al., “Identification of multiple risk variants for ankylosing spondylitis through high-density genotyping of immune-related loci,” Nat. Genet. (2013) 45(7):730-738; Remmers et al., “Genome-wide association study identifies variants in the MIIC class I, IL10, and IL23R-IL12RB2 regions associated with Behçet's disease,” Nat. Genet. (2010) 42:698-702. A genome-wide association study of 2,622 individuals with psoriasis identified associations between disease susceptibility and TYK2. Strange et al., “A genome-wide association study identifies new psoriasis susceptibility loci and an interaction between HLA-C and ERAP1,” Nat. Genet. (2010) 42:985-992. Knockout or tyrphostin inhibition of TYK2 significantly reduces both IL-23 and IL-22-induced dermatitis. Ishizaki et al., “Tyk2 is a therapeutic target for psoriasis-like skin inflammation,” Intl. Immunol. (2013), doi: 10.1093/intimm/dxt062.

TYK2 also plays a role in respiratory diseases such as asthma, chronic obstructive pulmonary disease (COPD), lung cancer, and cystic fibrosis. Goblet cell hyperplasia (GCH) and mucous hypersecretion is mediated by IL-13-induced activation of TYK2, which in turn activates STAT6. Zhang et al., “Docking protein Gab2 regulates mucin expression and goblet cell hyperplasia through TYK2/STAT6 pathway,” FASEB J. (2012) 26:1-11. Decreased TYK2 activity leads to protection of joints from collagen antibody-induced arthritis, a model of human rheumatoid arthritis. Mechanistically, decreased Tyk2 activity reduced the production of Th1/Th17-related cytokines and matrix metalloproteases, and other key markers of inflammation. Ishizaki et al., “Tyk2 deficiency protects joints against destruction in anti-type II collagen antibody-induced arthritis in mice,” Intl. Immunol. (2011) 23(9):575-582.

TYK2 knockout mice showed complete resistance in experimental autoimmune encephalomyelitis (EAE, an animal model of multiple sclerosis (MS)), with no infiltration of CD4 T cells in the spinal cord, as compared to controls, suggesting that TYK2 is essential to pathogenic CD4-mediated disease development in MS. Oyamada et al., “Tyrosine Kinase 2 Plays Critical Roles in the Pathogenic CD4 T Cell Responses for the Development of Experimental Autoimmune Encephalomyelitis,” J. Immunol. (2009) 183:7539-7546. This corroborates earlier studies linking increased TYK2 expression with MS susceptibility. Ban et al., “Replication analysis identifies TYK2 as a multiple sclerosis susceptibility factor,” Eur J. Hum. Genet. (2009) 17:1309-1313. Loss of function mutation in TYK2, leads to decreased demyelination and increased remyelination of neurons, further suggesting a role for TYK2 inhibitors in the treatment of MS and other CNS demyelination disorders.

TYK2 is the sole signaling messenger common to both IL-12 and IL-23. TYK2 knockout reduced methylated BSA injection-induced footpad thickness, imiquimod-induced psoriasis-like skin inflammation, and dextran sulfate sodium or 2,4,6-trinitrobenzene sulfonic acid-induced colitis in mice.

Joint linkage and association studies of various type I IFN signaling genes with systemic lupus erythematosus (SLE, an autoimmune disorder), showed a strong, and significant correlation between loss of function mutations to TYK2 and decreased prevalence of SLE in families with affected members. Sigurdsson et al., “Polymorphisms in the Tyrosine Kinase 2 and Interferon Regulatory Factor 5 Genes Are Associated with Systemic Lupus Erythematosus,” Am. J. Hum. Genet. (2005) 76:528-537. Genome-wide association studies of individuals with SLE versus an unaffected cohort showed highly significant correlation between the TYK2 locus and SLE. Graham et al., “Association of NCF2, IKZF1, RF8, IFIH1, and TYK2 with Systemic Lupus Erythematosus,” PLoS Genetics (2011) 7(10):e1002341.

TYK2 has been shown to play an important role in maintaining tumor surveillance and TYK2 knockout mice showed compromised cytotoxic T cell response, and accelerated tumor development. However, these effects were linked to the efficient suppression of natural killer (NK) and cytotoxic T lymphocytes, suggesting that TYK2 inhibitors would be highly suitable for the treatment of autoimmune disorders or transplant rejection. Although other JAK family members such as JAK3 have similar roles in the immune system, TYK2 has been suggested as a superior target because of its involvement in fewer and more closely related signaling pathways, leading to fewer off-target effects. Simma et al. “Identification of an Indispensable Role for Tyrosine Kinase 2 in CTL-Mediated Tumor Surveillance,” Cancer Res. (2009) 69:203-211.

However, paradoxically to the decreased tumor surveillance observed by Simma et al., studies in T-cell acute lymphoblastic leukemia (T-ALL) indicate that T-ALL is highly dependent on IL-10 via TYK2 via STAT1-mediated signal transduction to maintain cancer cell survival through upregulation of anti-apoptotic protein BCL2. Knockdown of TYK2, but not other JAK family members, reduced cell growth. Specific activating mutations to TYK2 that promote cancer cell survival include those to the FERM domain (G36D, S47N, and R425H), the JH2 domain (V731I), and the kinase domain (E957D and R1027H). However, it was also identified that the kinase function of TYK2 is required for increased cancer cell survival, as TYK2 enzymes featuring kinase-dead mutations (M978Y or M978F) in addition to an activating mutation (E957D) resulted in failure to transform. Sanda et al. “TYK2-STAT1-BCL2 Pathway Dependence in T-Cell Acute Lymphoblastic Leukemia,” Cancer Disc. (2013) 3(5):564-577.

Thus, selective degradation and/or inhibition of TYK2 has been suggested as a suitable target for patients with IL-10 and/or BCL2-addicted tumors, such as 70% of adult T-cell leukemia cases. Fontan et al. “Discovering What Makes STAT Signaling TYK in T-ALL,” Cancer Disc. (2013) 3:494-496.

TYK2 mediated STAT3 signaling has also been shown to mediate neuronal cell death caused by amyloid-β (Aβ) peptide. Decreased TYK2 phosphorylation of STAT3 following Aβ administration lead to decreased neuronal cell death, and increased phosphorylation of STAT3 has been observed in postmortem brains of Alzheimer's patients. Wan et al. “Tyk/STAT3 Signaling Mediates β-Amyloid-Induced Neuronal Cell Death: Implications in Alzheimer's Disease,” J. Neurosci. (2010) 30(20):6873-6881.

Inhibition of JAK-STAT signaling pathways is also implicated in hair growth, and the reversal of the hair loss associated with alopecia areata. Xing et al., “Alopecia areata is driven by cytotoxic T lymphocytes and is reversed by JAK inhibition,” Nat. Med. (2014) 20: 1043-1049; Harel et al., “Pharmacologic inhibition of JAK-STAT signaling promotes hair growth,” Sci. Adv. (2015) 1(9):e1500973.

Accordingly, compounds that degrade TYK2 and/or inhibit the activity of TYK2 are beneficial, especially those with selectivity over JAK2. Such compounds should deliver a pharmacological response that favorably treats one or more of the conditions described herein without the side-effects associated with the inhibition of JAK2.

Even though TYK2 inhibitors are known in the art, there is a continuing need to provide novel inhibitors having more effective or advantageous pharmaceutically relevant properties. For example, compounds with increased activity, selectivity over other JAK kinases (especially JAK2), and ADMET (absorption, distribution, metabolism, excretion, and/or toxicity) properties. Thus, in some embodiments, the present invention provides degraders of TYK2 which show selectivity over JAK2.

The activity of a compound utilized in this invention as a degrader of TYK2, or a mutant thereof, may be assayed in vitro, in vivo or in a cell line. In vitro assays include assays that determine inhibition of either the phosphorylation activity and/or the subsequent functional consequences, or ATPase activity of activated TYK2, or a mutant thereof. Alternate in vitro assays quantitate the ability of the inhibitor to bind to TYK2. Inhibitor binding may be measured by radiolabeling the inhibitor prior to binding, isolating the inhibitor/TYK2 complex and determining the amount of radiolabel bound. Alternatively, inhibitor binding may be determined by running a competition experiment where new inhibitors are incubated with TYK2 bound to known radioligands. Representative in vitro and in vivo assays useful in assaying a TYK2 degrader and/or inhibitor include those described and disclosed in, e.g., each of which is herein incorporated by reference in its entirety.

Provided compounds are degraders and/or inhibitors of TYK2 and are therefore useful for treating one or more disorders associated with activity of TYK2 or mutants thereof. Thus, in certain embodiments, the present invention provides a method for treating a TYK2-mediated disorder comprising the step of administering to a patient in need thereof a compound of the present invention, or pharmaceutically acceptable composition thereof.

As used herein, the term “TYK2-mediated” disorders, diseases, and/or conditions as used herein means any disease or other deleterious condition in which TYK2 or a mutant thereof is known to play a role. Accordingly, another embodiment of the present invention relates to treating or lessening the severity of one or more diseases in which TYK2, or a mutant thereof, is known to play a role. Such TYK2-mediated disorders include but are not limited to autoimmune disorders, inflammatory disorders, proliferative disorders, endocrine disorders, neurological disorders and disorders associated with transplantation.

In some embodiments, the present invention provides a method for treating one or more disorders, wherein the disorders are selected from autoimmune disorders, inflammatory disorders, proliferative disorders, endocrine disorders, neurological disorders, and disorders associated with transplantation, said method comprising administering to a patient in need thereof, a pharmaceutical composition comprising an effective amount of a compound of the present invention, or a pharmaceutically acceptable salt thereof.

In some embodiments, the disorder is an autoimmune disorder. In some embodiments the disorder is selected from type 1 diabetes, systemic lupus erythematosus, multiple sclerosis, psoriasis, Behçet's disease, POEMS syndrome, Crohn's disease, ulcerative colitis, and inflammatory bowel disease.

In some embodiments, the disorder is an inflammatory disorder. In some embodiments, the inflammatory disorder is rheumatoid arthritis, asthma, chronic obstructive pulmonary disease, psoriasis, hepatomegaly, Crohn's disease, ulcerative colitis, inflammatory bowel disease.

In some embodiments, the disorder is a proliferative disorder. In some embodiments, the proliferative disorder is a hematological cancer. In some embodiments the proliferative disorder is a leukemia. In some embodiments, the leukemia is a T-cell leukemia. In some embodiments the T-cell leukemia is T-cell acute lymphoblastic leukemia (T-ALL). In some embodiments the proliferative disorder is polycythemia vera, myelofibrosis, essential or thrombocytosis.

In some embodiments, the disorder is an endocrine disorder. In some embodiments, the endocrine disorder is polycystic ovary syndrome, Crouzon's syndrome, or type 1 diabetes.

In some embodiments, the disorder is a neurological disorder. In some embodiments, the neurological disorder is Alzheimer's disease.

In some embodiments the proliferative disorder is associated with one or more activating mutations in TYK2. In some embodiments, the activating mutation in TYK2 is a mutation to the FERM domain, the JH2 domain, or the kinase domain. In some embodiments the activating mutation in TYK2 is selected from G36D, S47N, R425H, V731I, E957D, and R1027H.

In some embodiments, the disorder is associated with transplantation. In some embodiments the disorder associated with transplantation is transplant rejection, or graft versus host disease.

In some embodiments the disorder is associated with type I interferon, IL-10, IL-12, or IL-23 signaling. In some embodiments the disorder is associated with type I interferon signaling. In some embodiments the disorder is associated with IL-10 signaling. In some embodiments the disorder is associated with IL-12 signaling. In some embodiments the disorder is associated with IL-23 signaling.

Compounds of the invention are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Compounds of the invention may also be used for the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleroderma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine opthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minal change nephropathy), chronic granulomatous disease, endometriosis, leptospiriosis renal disease, glaucoma, retinal disease, ageing, headache, pain, complex regional pain syndrome, cardiac hypertrophy, musclewasting, catabolic disorders, obesity, fetal growth retardation, hyperchlolesterolemia, heart disease, chronic heart failure, mesothelioma, anhidrotic ecodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments the inflammatory disease which can be treated according to the methods of this invention is selected from acute and chronic gout, chronic gouty arthritis, psoriasis, psoriatic arthritis, rheumatoid arthritis, Juvenile rheumatoid arthritis, Systemic juvenile idiopathic arthritis (SJIA), Cryopyrin Associated Periodic Syndrome (CAPS), and osteoarthritis.

In some embodiments the inflammatory disease which can be treated according to the methods of this invention is a Th1 or Th17 mediated disease. In some embodiments the Th17 mediated disease is selected from Systemic lupus erythematosus, Multiple sclerosis, and inflammatory bowel disease (including Crohn's disease or ulcerative colitis).

In some embodiments the inflammatory disease which can be treated according to the methods of this invention is selected from Sjogren's syndrome, allergic disorders, osteoarthritis, conditions of the eye such as ocular allergy, conjunctivitis, keratoconjunctivitis sicca and vernal conjunctivitis, and diseases affecting the nose such as allergic rhinitis.

Furthermore, the invention provides the use of a compound according to the definitions herein, or a pharmaceutically acceptable salt, or a hydrate or solvate thereof for the preparation of a medicament for the treatment of an autoimmune disorder, an inflammatory disorder, or a proliferative disorder, or a disorder commonly occurring in connection with transplantation.

In some embodiments, the present invention provides a method of treating pain in a patient in need thereof, the method comprising administering to the patient a compound of the present invention or a therapeutically acceptable salt thereof.

In some embodiments, the present invention provides a method of treating inflammation in a patient in need thereof, the method comprising administering to the patient a compound of the present invention or a therapeutically acceptable salt thereof.

In some embodiments, the present invention provides a method of treating tissue damage in a patient in need thereof, the method comprising administering to the patient a compound of the present invention or a therapeutically acceptable salt thereof.

In some embodiments, the present invention provides a method of treating arthritis in a patient in need thereof, the method comprising administering to the patient a compound of the present invention or a therapeutically acceptable salt thereof.

It is believed that a provided compound or a pharmaceutically acceptable salt thereof may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

In some embodiments, the compounds of the invention are used to treat the TYK2-mediated disorder.

In some embodiments, the compounds of the invention are used to treat the TYK2-mediated disorder, wherein the disorder is an inflammatory disorder, a proliferative disorder, an endocrine disorder, a neurological disorder, or a disorder associated with transplantation.

In some embodiments, the compounds of the invention are used to treat the TYK2-mediated disorder, wherein the disorder is an autoimmune disorder selected from type 1 diabetes, ankylosing spondylitis, cutaneous lupus erythematosus, systemic lupus erythematosus, multiple sclerosis, systemic sclerosis, psoriasis, Crohn's disease, ulcerative colitis, and inflammatory bowel disease.

In some embodiments, the compounds of the invention are used to treat the TYK2-mediated disorder, wherein the disorder is an inflammatory disorder selected from rheumatoid arthritis, asthma, chronic obstructive pulmonary disease, psoriasis, Crohn's disease, ulcerative colitis, and inflammatory bowel disease.

In some embodiments, the compounds of the invention are used to treat the TYK2-mediated disorder, wherein the disorder is a proliferative disorder selected from a hematological cancer, polycythemia vera, myelofibrosis, essential thrombocythemia, and thrombocytosis.

In some embodiments, the compounds of the invention are used to treat the TYK2-mediated disorder, wherein the disorder is an endocrine disorder selected from polycystic ovary syndrome, Crouzon's syndrome, and type 1 diabetes.

In some embodiments, the compounds of the invention are used to treat the TYK2-mediated disorder, wherein the disorder is a neurological disorder selected from Alzheimer's disease, Parkinson's disease, amyotrophic lateral sclerosis, Huntington's disease, cerebral ischemia, and neurodegenerative disease caused by traumatic injury, glutamate neurotoxicity and hypoxia.

In some embodiments, the compounds of the invention are used to treat the TYK2-mediated disorder, wherein the disorder is associated with transplantation selected from transplant rejection and graft versus host disease.

In some embodiments, the compounds of the invention are used to a subject that is human.

Co-Administration with One or More Other Therapeutic Agent(s)

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinirole, pramipexole, bromocriptine, pergolide, trihexyphenidyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen within greater than 24 hours apart.

In one embodiment, the present invention provides a composition comprising a provided compound and one or more additional therapeutic agents. The therapeutic agent may be administered together with a provided compound, or may be administered prior to or following administration of a provided compound. Suitable therapeutic agents are described in further detail below. In certain embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating an inflammatory disease, disorder or condition by administering to a patient in need thereof a provided compound and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating gout comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol and febuxostat (Uloric®).

In another embodiment, the present invention provides a method of treating rheumatoid arthritis comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating lupus comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating inflammatory bowel disease comprising administering to a patient in need thereof a provided compound and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a provided compound and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-bid®, Uniphyl®, Theo-24®) and aminophylline, and IgE antibodies such as omalizumab (Xolair®).

In some embodiments, the present invention provides a method of treating COPD comprising administering to a patient in need thereof a provided compound and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®,

In some embodiments, the present invention provides a method of treating HIV comprising administering to a patient in need thereof a provided compound and one or more additional therapeutic agents selected from nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), and combinations thereof.

In another embodiment, the present invention provides a method of treating a hematological malignancy comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, an alternative TYK2 inhibitor, a PI3K inhibitor, a SYK inhibitor, and combinations thereof.

In another embodiment, the present invention provides a method of treating a solid tumor comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, an alternative TYK2 inhibitor, a PI3K inhibitor, a SYK inhibitor, and combinations thereof.

In another embodiment, the present invention provides a method of treating a hematological malignancy comprising administering to a patient in need thereof a provided compound and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, the present invention provides a method of treating multiple myeloma comprising administering to a patient in need thereof a provided compound and one or more additional therapeutic agents selected from bortezomib (Velcade®), and dexamethasone (Decadron®), a hedgehog signaling inhibitor, a BTK inhibitor, a JAK/pan-JAK inhibitor, an alternative TYK2 inhibitor, a PI3K inhibitor, a SYK inhibitor in combination with lenalidomide (Revlimid®).

In another embodiment, the present invention provides a method of treating Waldenström's macroglobulinemia comprising administering to a patient in need thereof a provided compound and one or more additional therapeutic agents selected from chlorambucil (Leukeran®), cyclophosphamide (Cytoxan®, Neosar®), fludarabine (Fludara®), cladribine (Leustatin®), rituximab (Rituxan®), a hedgehog signaling inhibitor, a BTK inhibitor, a JAK/pan-JAK inhibitor, an alternative TYK2 inhibitor, a PI3K inhibitor, and a SYK inhibitor.

In some embodiments, one or more other therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In some embodiments, one or more other therapeutic agent is a Poly ADP ribose polymerase (PARP) inhibitor. In some embodiments, a PARP inhibitor is selected from olaparib (Lynparza®, AstraZeneca); rucaparib (Rubraca®, Clovis Oncology); niraparib (Zejula®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (Zolinza®, Merck); romidepsin (Istodax®, Celgene); panobinostat (Farydak®, Novartis); belinostat (Beleodaq®, Spectrum Pharmaceuticals); entinostat (SNDX-275, Syndax Pharmaceuticals) (NCT00866333); and chidamide (Epidaza®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (Ibrance®, Pfizer); ribociclib (Kisqali®, Novartis); abemaciclib (Ly2835219, Eli Lilly); and trilaciclib (G1T28, G1 Therapeutics).

In some embodiments, one or more other therapeutic agent is a folic acid inhibitor. Approved folic acid inhibitors useful in the present invention include pemetrexed (Alimta®, Eli Lilly).

In some embodiments, one or more other therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In some embodiments, one or more other therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In some embodiments, one or more other therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present invention include enzalutamide (Xtandi®, Astellas/Medivation); approved inhibitors of androgen synthesis include abiraterone (Zytiga®, Centocor/Ortho); approved antagonist of gonadotropin-releasing hormone (GnRH) receptor (degaralix, Firmagon®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (Evista®, Eli Lilly).

In some embodiments, one or more other therapeutic agent is an inhibitor of bone resorption. An approved therapeutic which inhibits bone resorption is Denosumab (Xgeva®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (Zometa®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFI3). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGFI3 trap compound (NCT02699515); and (NCT02517398). M7824 is comprised of a fully human IgG1 antibody against PD-L1 fused to the extracellular domain of human TGF-beta receptor II, which functions as a TGFI3 “trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

In some embodiments, one or more other therapeutic agent is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx;

MEK inhibitors such as ARRY142886 from Array BioPharma, AZd6244 from AstraZeneca, PD181461 from Pfizer and leucovorin.

In some embodiments, the present invention provides a method of treating Alzheimer's disease comprising administering to a patient in need thereof a provided compound and one or more additional therapeutic agents selected from donepezil (Aricept®), rivastigmine (Excelon®), galantamine (Razadyne®), tacrine (Cognex®), and memantine (Namenda®).

In some embodiments, one or more other therapeutic agent is a taxane compound, which causes disruption of microtubules, which are essential for cell division. In some embodiments, a taxane compound is selected from paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), cabazitaxel (Jevtana®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In some embodiments, one or more other therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

Other kinase inhibitors and VEGF-R antagonists that are in development and may be used in the present invention include tivozanib (Aveo Pharmaceuticals); vatalanib (Bayer/Novartis); lucitanib (Clovis Oncology); dovitinib (TK1258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In another embodiment, the present invention provides a method of treating organ transplant rejection or graft vs. host disease comprising administering to a patient in need thereof a provided compound and one or more additional therapeutic agents selected from a steroid, cyclosporin, FK506, rapamycin, a hedgehog signaling inhibitor, a BTK inhibitor, a JAK/pan-JAK inhibitor, an alternative TYK2 inhibitor, a PI3K inhibitor, and a SYK inhibitor.

In another embodiment, the present invention provides a method of treating or lessening the severity of a disease comprising administering to a patient in need thereof a provided compound and a BTK inhibitor, wherein the disease is selected from inflammatory bowel disease, arthritis, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type 1 hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleroderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a provided compound and a PI3K inhibitor, wherein the disease is selected from a cancer, a neurodegenerative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a provided compound and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracnosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleredoma, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments, one or more other therapeutic agent is a phosphatidylinositol 3 kinase (PI3K) inhibitor. In some embodiments, a PI3K inhibitor is selected from idelalisib (Zydelig®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

The compounds and compositions, according to the method of the present invention, may be administered using any amount and any route of administration effective for treating or lessening the severity of a cancer, an autoimmune disorder, a proliferative disorder, an inflammatory disorder, a neurodegenerative or neurological disorder, schizophrenia, a bone-related disorder, liver disease, or a cardiac disorder. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of inhibiting protein kinase activity or degrading a protein kinase in a biological sample comprising the step of contacting said biological sample with a compound of this invention, or a composition comprising said compound.

According to another embodiment, the invention relates to a method of inhibiting or degrading TYK2, or a mutant thereof, activity in a biological sample comprising the step of contacting said biological sample with a compound of this invention, or a composition comprising said compound.

The term “biological sample”, as used herein, includes, without limitation, cell cultures or extracts thereof, biopsied material obtained from a mammal or extracts thereof, and blood, saliva, urine, feces, semen, tears, or other body fluids or extracts thereof.

Inhibition and/or degradation of a TYK2 protein, or a protein selected from TYK2, or a mutant thereof, activity in a biological sample is useful for a variety of purposes that are known to one of skill in the art. Examples of such purposes include, but are not limited to, blood transfusion, organ-transplantation, biological specimen storage, and biological assays.

Another embodiment of the present invention relates to a method of degrading a protein kinase and/or inhibiting protein kinase activity in a patient comprising the step of administering to said patient a compound of the present invention, or a composition comprising said compound.

According to another embodiment, the invention relates to a method of degrading and/or inhibiting TYK2, or a mutant thereof, activity in a patient comprising the step of administering to said patient a compound of the present invention, or a composition comprising said compound. In other embodiments, the present invention provides a method for treating a disorder mediated by TYK2, or a mutant thereof, in a patient in need thereof, comprising the step of administering to said patient a compound according to the present invention or pharmaceutically acceptable composition thereof. Such disorders are described in detail herein.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

A compound of the current invention may also be used to advantage in combination with other antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, rogletimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

In some embodiments, one or more other therapeutic agent is an mTOR inhibitor, which inhibits cell proliferation, angiogenesis and glucose uptake. In some embodiments, an mTOR inhibitor is everolimus (Afinitor®, Novartis); temsirolimus (Torisel®, Pfizer); and sirolimus (Rapamune®, Pfizer).

In some embodiments, one or more other therapeutic agent is an aromatase inhibitor. In some embodiments, an aromatase inhibitor is selected from exemestane (Aromasin®, Pfizer); anastazole (Arimidex®, AstraZeneca) and letrozole (Femara®, Novartis).

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecin and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophyllotoxins etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; isis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR1 ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, ELI, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

Compounds which target, decrease or inhibit the activity of a protein or lipid phosphatase are e.g. inhibitors of phosphatase 1, phosphatase 2A, or CDC25, such as okadaic acid or a derivative thereof.

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3K13, PI3K-C2α, PI3K-C213, PI3K-C2γ, Vps34, p110-α, p110-13, p110-γ, p110-δ, p85-α, p85-13, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™)); carfilzomib (Kyprolis®, Amgen); and ixazomib (Ninlaro®, Takeda), and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4th Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclomethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of one or more other therapeutic agent present in the compositions of this invention may be no more than the amount that would normally be administered in a composition comprising that therapeutic agent as the only active agent. Preferably the amount of one or more other therapeutic agent in the presently disclosed compositions will range from about 50% to 100% of the amount normally present in a composition comprising that agent as the only therapeutically active agent. In some embodiments, one or more other therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is provided for dosing per the FDA label insert.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTJ3R, LIGHT, DcR3, HVEM, VEGI/TL1A, TRAMP/DR3, EDAR, EDA1, XEDAR, EDA2, TNFR1, Lymphotoxin α/TNFJ3, TNFR2, TNFα, LTJ3R, Lymphotoxin α1J32, FAS, FASL, RELT, DR6, TROY, NGFR.

In some embodiments, an immuno-oncology agent is a cytokine that inhibits T cell activation (e.g., IL-6, IL-10, TGF-J3, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIRI, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MEDI4736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intetumumab, ipilimumab, isatuximab, lambrolizumab, MED14736, MPDL3280A, nivolumab, obinutuzumab, ocaratuzumab, ofatumumab, olaratumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (Opdivo®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+ (Th17) and CD8+ (Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams et al., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams et al. In some embodiments, an immuno-oncology agent is a small molecule targeting an immuno-oncology target selected from those listed in Table 1 of Jerry L. Adams ET. AL. In some embodiments, an immuno-oncology agent is a small molecule agent selected from those listed in Table 1 of Jerry L. Adams et al.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTE®) antibody constructs can mediate bystander tumor cell killing”, PLoS ONE 12(8): e0183390, the contents of which is incorporated herein by reference in its entirety. In some embodiments, an immuno-oncology agent is a bispecific T cell engager (BiTE®) antibody construct. In some embodiments, a bispecific T cell engager (BiTE®) antibody construct is a CD19/CD3 bispecific antibody construct. In some embodiments, a bispecific T cell engager (BiTE®) antibody construct is an EGFR/CD3 bispecific antibody construct. In some embodiments, a bispecific T cell engager (BiTE®) antibody construct activates T cells. In some embodiments, a bispecific T cell engager (BiTE®) antibody construct activates T cells, which release cytokines inducing upregulation of intercellular adhesion molecule 1 (ICAM-1) and FAS on bystander cells. In some embodiments, a bispecific T cell engager (BiTE®) antibody construct activates T cells which result in induced bystander cell lysis. In some embodiments, the bystander cells are in solid tumors. In some embodiments, the bystander cells being lysed are in proximity to the BiTE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8+(αβ) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PDL1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); and atezolizumab (anti-PD-L1 antibody, Tecentriq®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirilumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that may be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

In some embodiments, TYK2 inhibition/degradation can significantly enhance CDN-induced STING signaling and antitumor immunity (Pei et al., Can. Lett. 2019, 450:110).

Checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that may be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

### EXAMPLES

The following examples are provided to further describe some of the embodiments disclosed herein. The examples are intended to illustrate, not to limit, the disclosed embodiments.

### Example 1. Preparation of Intermediates

The preparation methods for the intermediates are shown below

### Example 1.1. Preparation of 5-Bromo-3-methyl-1H-benzimidazol-2-one (Intermediate A)

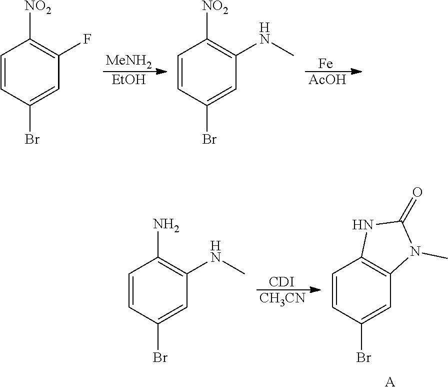

### Step 1—5-Bromo-N-methyl-2-nitro-aniline

4-bromo-2-fluoro-1-nitro-benzene (230 g, 1.05 mol, CAS #321-23-3) was added to a solution of methylamine in tetrahydrofuran (2 M, 1.51 L). The mixture was stirred at 15° C. for 10 minutes. On completion, the mixture was diluted with H₂O (250 mL) and extracted with EtOAc (3×300 mL). The combined organic layers were washed with brine (300 mL), dried over Na₂SO₄, filtered and concentrated in vacuo to give the title compound (200 g, 83% yield) as a yellow solid. ¹H NMR (400 MHz, DMSO-d₆) δ 8.22 (s, 1H), 7.98 (d, J=9.2 Hz, 1H), 7.16 (d, J=1.6 Hz, 1H), 6.82 (dd, J=8.4, 1.6 Hz, 1H), 2.95 (d, J=4.8 Hz, 3H).

### Step 2—4-Bromo-N2-methyl-benzene-1,2-diamine

To a mixture of 5-bromo-N-methyl-2-nitro-aniline (200 g, 865 mmol) in EtOAc (1 L) and H₂O (500 mL) was added AcOH (1.00 L). The mixture was warmed to 50° C., and then Fe (174 g, 3.11 mol) was added to the reaction mixture. After that, the reaction mixture was stirred at 80° C. for 6 hours. On completion, the mixture was filtered through celite. The filtrate was concentrated in vacuo and the residue was diluted with H₂O (250 mL) and extracted with EtOAc (3×300 mL). The combined organic layers were washed with aq.NaHCO₃ and brine (300 mL), dried over Na₂SO₄, filtered and concentrated in vacuo. The residue was purified by flash silica gel chromatography to give the title compound (130 g, 75% yield) as black oil. ¹H NMR (400 MHz, DMSO-d₆) δ 6.55-6.52 (m, 1H), 6.48-6.45 (m, 1H), 6.43-6.42 (m, 1H), 4.89-4.88 (m, 1H), 4.61 (s, 2H), 2.70 (d, J=4.0 Hz, 3H).

### Step 3—5-Bromo-3-methyl-1H-benzimidazol-2-one

To a solution of 4-bromo-N2-methyl-benzene-1,2-diamine (110 g, 547 mmol) in CH₃CN (1.3 L) was added CDI (177 g, 1.09 mol). The mixture was stirred at 80° C. for 6 hours under N₂. On completion, the mixture was concentrated in vacuo. The mixture was diluted with H₂O (1.0 L) and filtered. The filter cake was washed with water (3×200 mL) and dried in vacuo to give the title compound (106 g, 85% yield) as a white solid. ¹H NMR (400 MHz, DMSO-d₆) δ 11.00 (s, 1H), 7.33 (s, 1H), 7.13 (d, J=8.0 Hz, 1H), 6.92 (d, J=8.0 Hz, 1H), 3.27 (s, 3H).

### Example 1.2. Preparation of [1-[(4-Methoxyphenyl)methyl]-2,6-dioxo-3-piperidyl] trifluoromethanesulfonate (Intermediate B)

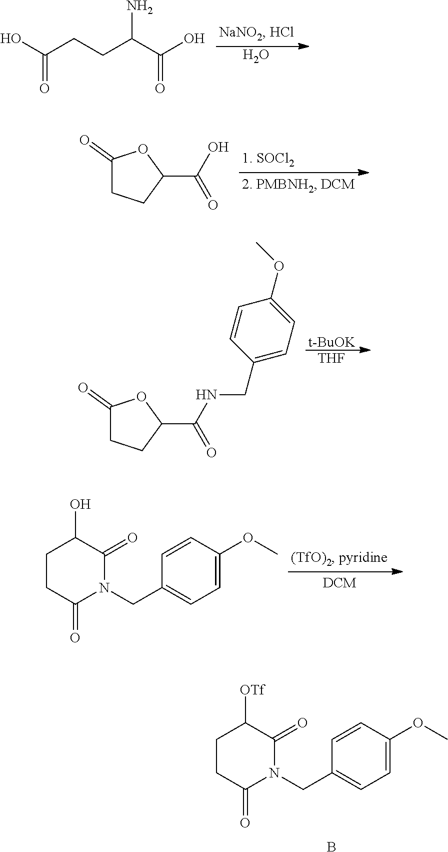

### Step 1—5-Oxotetrahydrofuran-2-carboxylic Acid

To a solution of 2-aminopentanedioic acid (210 g, 1.43 mol, CAS #617-65-2) in H₂O (800 mL) and HCl (12 M, 210 mL) was added a solution of NaNO₂ (147 g, 2.13 mol) in H₂O (400 mL) at −5° C. The mixture was stirred at 15° C. for 12 hrs. On completion, the mixture was concentrated and then dissolved in EA (500 mL) and filtered and washed with EA (3×100 mL). The filtrate and washed solution were dried over Na₂SO₄, filtered and concentrated in vacuo to give the title compound (200 g, crude) as yellow oil. ¹H NMR (400 MHz, CDCl₃) δ 6.43 (s, 1H), 5.02-4.95 (m, 1H), 2.67-2.38 (m, 4H)

### Step 2—N-[(4-methoxyphenyl)methyl]-5-oxo-tetrahydrofuran-2-carboxamide

To 5-oxotetrahydrofuran-2-carboxylic acid (120 g, 922 mmol) was added SOCl₂ (246 g, 2.07 mol) at 0° C. slowly. The mixture was stirred at 85° C. for 3 hrs, and then the mixture was stirred at 15° C. for 6 hrs. The mixture was concentrated in vacuo. The residue was dissolved in dry DCM (1 L) at 0° C. under N₂. After that a solution of Et₃N (187 g, 1.84 mol) and 4-methoxybenzylamine (101 g, 738 mmol) in DCM (400 mL) was added, then the mixture was stirred at 15° C. for 3 hrs. On completion, water (600 mL) was added and the mixture was extracted with DCM (3×300 mL). The combined organic phase was washed with 0.5 M HCl (500 mL), brine (500 mL), dried over with anhydrous sodium sulfate and filtered. The filtrate was concentrated in vacuo and the residue was purified by flash silica gel chromatography (PE:EA=1:1) to give the title compound (138 g, 60% yield) as a yellow solid. ¹H NMR (400 MHz, CDCl₃) δ 7.22-7.20 (d, J=8.0, 1H), 6.89-6.87 (d, J=8.0, 1H), 4.90-4.86 (m, 1H), 4.47-4.4.36 (m, 2H) 3.81 (s, 3H), 2.67-2.64 (m, 1H), 2.59-2.54 (m, 2H), 2.40-2.38 (m, 1H); LC-MS (ESI⁺) m/z 272.0 (M+Na)⁺.

### Step 3—3-Hydroxy-1-[(4-methoxyphenyl)methyl]piperidine-2,6-dione

A solution of N-[(4-methoxyphenyl)methyl]-5-oxo-tetrahydrofuran-2-carboxamide (138 g, 553 mmol) in anhydrous THF (1500 mL) was cooled to −78° C. Then, t-BuOK (62.7 g, 559 mmol) in a solution of anhydrous THF (1000 mL) was added dropwise slowly at −78° C. under nitrogen atmosphere. The resulting reaction mixture was stirred at −40° C. for 1 hr. On completion, the reaction mixture was quenched with saturated NH₄Cl solution (100 mL). The mixture was extracted with ethyl acetate (3×1500 mL). The combined organic layer was washed with brine (300 mL), dried over anhydrous sodium sulfate, filtered and the filtrate was concentrated in vacuo. The residue was purified by silica gel chromatography (PE:EA=1:1) to give the title compound (128 g, 92% yield) as a white solid. ¹H NMR (400 MHz, CDCl₃) δ 7.39-7.32 (m, 2H), 6.89-6.81 (m, 2H), 4.91 (s, 2H), 4.17-4.11 (m, 1H), 3.80 (s, 3H), 3.54 (s, 1H), 2.98-2.87 (m, 1H), 2.73-2.60 (m, 1H), 2.26-2.20 (m, 1H), 1.80 (dq, J=4.8, 13.1 Hz, 1H).

### Step 4—[1-[(4-Methoxyphenyl)methyl]-2,6-dioxo-3-piperidyl] trifluoromethanesulfonate

To a solution of 3-hydroxy-1-[(4-methoxyphenyl) methyl] piperidine-2, 6-dione (43.0 g, 173 mmol) and pyridine (27.3 g, 345 mmol) in DCM (500 mL) was added trifluoromethylsulfonyl trifluoromethanesulfonate (73.0 g, 258 mmol) dropwise at 0° C. The mixture was stirred at −10° C. for 1.5 hours under N₂. On completion, the mixture was concentrated in vacuo. The residue was purified by column chromatography on silica gel (PE:EA=20:1/8:1) to give the title compound (45.0 g, 68% yield) as light yellow gum. ¹H NMR (400 MHz, CDCl₃) δ 7.36 (d, J=8.4 Hz, 2H), 6.85-6.82 (m, 2H), 5.32-5.28 (m, 1H), 4.91 (s, 2H), 3.79 (s, 3H), 3.02-2.97 (m, 1H), 2.79-2.74 (m, 1H), 2.41-2.35 (m, 2H).

### Example 1.3. Preparation of 3-(5-bromo-3-methyl-2-oxo-benzimidazol-1-yl)piperidine-2,6-dione (Intermediate C)

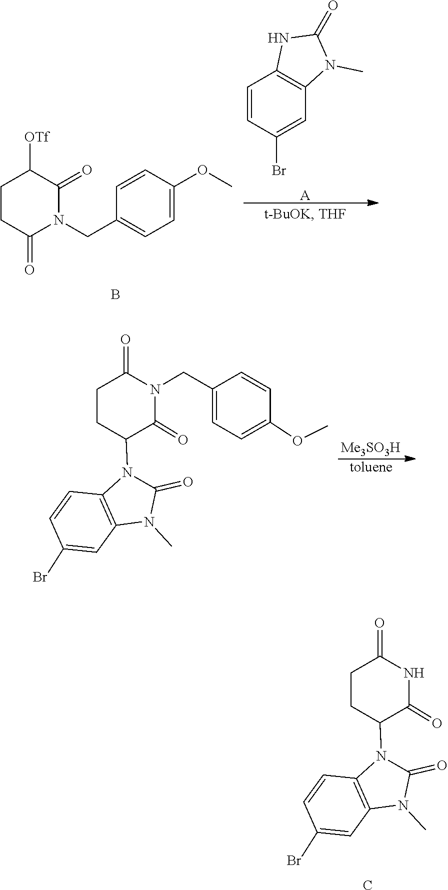

### Step 1—3-(5-Bromo-3-methyl-2-oxo-benzimidazol-1-yl)-1-[(4-methoxyphenyl)methyl]piperidine-2,6-dione

To a solution of 5-bromo-3-methyl-1H-benzimidazol-2-one (4.90 g, 21.6 mmol, Intermediate A) in THF (300 mL) was added t-BuOK (3.63 g, 32.3 mmol) at 0° C. The mixture was stirred at 0-10° C. for 1 hour under N₂. Then a solution of [1-[(4-methoxyphenyl)methyl]-2, 6-dioxo-3-piperidyl] trifluoromethanesulfonate (9.87 g, 25.9 mmol, Intermediate B) in THF (100 mL) was added to the reaction mixture at 0-10° C. during 30 minutes. The mixture was stirred at 0-10° C. for 30 minutes under N₂. An additional solution of [1-[(4-methoxyphenyl)methyl]-2, 6-dioxo-3-piperidyl] trifluoromethanesulfonate (2.47 g, 6.47 mmol) in THF (20 mL) was added to the reaction mixture at 0-10° C. dropwise. The mixture was then stirred at 0-10° C. for another 30 minutes under N₂. On completion, the reaction was quenched water (400 mL) and extracted with EA (3×200 mL). The combined organic layer was concentrated in vacuo. The residue was triturated with EA (80 mL) and filtered. The filter cake was collected and dried in vacuo to give the title compound (6.70 g, 67% yield) as light yellow solid. The filtrate was also concentrated in vacuo and the residue was purified by column chromatography to give another batch title compound (1.80 g, 18% yield) as light yellow solid. ¹H NMR (400 MHz, DMSO-d₆) δ 7.47 (d, J=1.6 Hz, 1H), 7.21-7.16 (m, 3H), 7.01 (d, J=8.0 Hz, 1H), 6.85 (d, J=8.8 Hz, 2H), 5.55-5.51 (m, 1H), 4.84-4.73 (m, 2H), 3.72 (s, 3H), 3.33 (s, 3H), 3.04-3.00 (m, 1H), 2.83-2.67 (m, 2H), 2.07-2.05 (m, 1H).

### Step 2—3-(5-Bromo-3-methyl-2-oxo-benzimidazol-1-yl)piperidine-2,6-dione

To a mixture of 3-(5-bromo-3-methyl-2-oxo-benzimidazol-1-yl)-1-[(4-methoxyphenyl)methyl] piperidine-2,6-dione (8.50 g, 18.6 mmol) in toluene (50 mL) was added methanesulfonic acid (33.8 g, 351 mmol, 25 mL) at room temperature (15° C.). The mixture was stirred at 120° C. for 2 hours. On completion, the reaction mixture was cooled to room temperature and concentrated in vacuo. The residue was poured into ice/water (200 mL), and extracted with EA (3×100 mL). The combined organic layer was washed with brine (50 mL), dried over Na₂SO₄, filtered and concentrated in vacuo. The residue was triturated with EA (80 mL) and filtered. The filtrate cake was collected and dried in vacuo to give the title compound (4.20 g, 67% yield) as off-white solid. ¹H NMR (400 MHz, DMSO-d₆) δ 11.12 (s, 1H), 7.47 (d, J=2.0 Hz, 1H), 7.22 (d, J=8.4 Hz, 1H), 7.10 (d, J=8.4 Hz, 1H), 5.40-5.35 (m, 1H), 2.34 (s, 3H), 2.92-2.88 (m, 1H), 2.71-2.60 (m, 2H), 2.03-1.99 (m, 1H).

### Example 1.4. Preparation of Tert-butyl (10-bromodecyl)carbamate (Intermediate D)

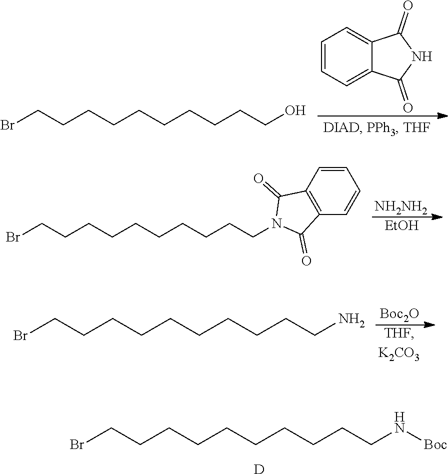

### Step 1—2-(10-bromodecyl)isoindoline-1,3-dione

To a solution of 10-bromodecan-1-ol (30 g, 126.5 mmol, CAS #53463-68-6) and isoindoline-1,3-dione (22.33 g, 151.8 mmol, CAS #85-41-6) in THF (350 mL) was added PPh₃ (48.11 g, 183.4 mmol) and DIAD (37.09 g, 183.4 mmol, 35.66 mL) at 0° C. The mixture was then stirred at 20° C. for 15 h. On completion, the reaction mixture was filtered and the filter cake was washed with EA (40 mL). The filtrate was poured into H₂O (300 mL) and extracted with ethyl acetate (300 mL×2). The organic phase was washed with brine (100 mL×2), dried over anhydrous Na₂SO₄, concentrated in vacuo to give a residue. The resulting residue was purified by silica gel column chromatography (PE:EA=10:1-5:1) to give the title compound (35 g, 76% yield) as white solid. ¹H NMR (400 MHz, CDCl₃) δ 7.88-7.72 (m, 2H), 7.69-7.55 (m, 2H), 3.69-3.54 (m, 2H), 3.33 (t, J=6.8 Hz, 2H), 1.77 (quin, J=7.2 Hz, 2H), 1.60 (quin, J=7.2 Hz, 2H), 1.39-1.11 (m, 12H).

### Step 2—10-Bromodecan-1-amine

To a solution of 2-(10-bromodecyl)isoindoline-1,3-dione (15 g, 41 mmol) in EtOH (150 mL) was added NH₂NH₂·H₂O (4.1 g, 80 mmol, 3.98 mL) at 15° C. The mixture was then stirred at 70° C. for 1.5 h. On completion, the mixture was cooled to rt and slowly added to 1 M HCl (100 mL). The mixture was filtered, then the filtrate was extracted with EA (500 mL×4). The combined organic layer was washed with brine (100 mL×2), dried over Na₂SO₄, filtered and concentrated under reduced pressure to give the title compound (9.7 g) as light yellow solid. LC-MS (ESI⁺) m/z 235.8, 237.8 (M+1)⁺.

### Step 3—Tert-butyl (10-bromodecyl)carbamate

To a solution of 10-bromodecan-1-amine (9.7 g, 41 mmol) in THF (150 mL) was added K₂CO₃ (11.35 g, 82.14 mmol) and Boc₂O (17.93 g, 82.14 mmol, 18.87 mL) at 10° C. The mixture was then stirred at 25° C. for 16 h. On completion, the reaction mixture was filtered and the filtrate concentrated to give an oil. The resulting oil was purified by silica gel column chromatography (PE:EA=100:1-10:1) to give the title compound (7.2 g, 52% yield) as white solid. ¹H NMR (400 MHz, CDCl₃) δ 4.50 (br s, 1H), 3.50-3.34 (m, 2H), 3.11 (q, J=6.4 Hz, 2H), 1.91-1.81 (m, 2H), 1.50-1.39 (m, 12H), 1.36-1.24 (m, 11H). MS (ESI⁺) m/z 280.0, 282.0 (M+1-56)⁺.

### Example 1.5. Preparation of 3-(5-(10-aminodecyl)-3-methyl-2-oxo-2,3-dihydro-1H-benzo[d]imidazol-1-yl)piperidine-2,6-dione (Intermediate E)

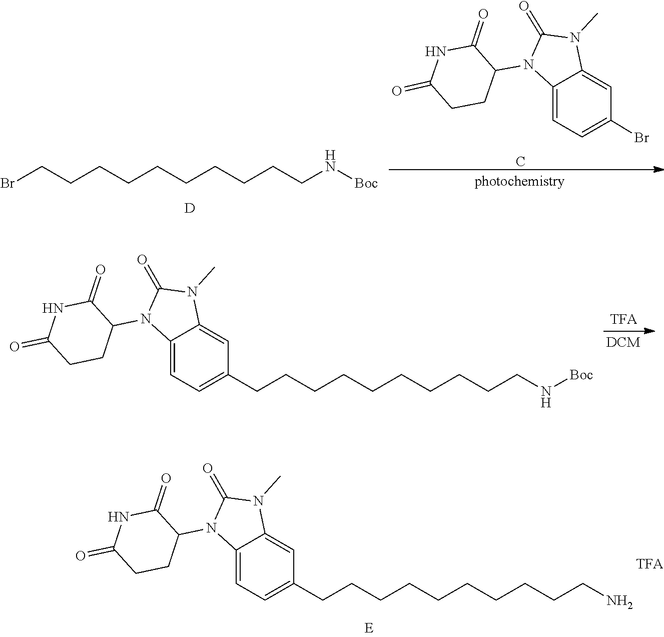

### Step 1—3-[3-[1-(2,6-Dioxo-3-piperidyl)-3-methyl-2-oxo-benzimidazol-5-yl]propoxy]propanoic Acid

To an vial equipped with a stir bar was added 3-(5-bromo-3-methyl-2-oxo-benzimidazol-1-yl)piperidine-2,6-dione (5 g, 14.79 mmol, Intermediate C), tert-butyl N-(10-bromodecyl)carbamate (6.46 g, 19.22 mmol, Intermediate D), bis(trimethylsilyl)silyl-trimethyl-silane (3.68 g, 14.8 mmol, 4.56 mL), bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (1.66 g, 1.48 mmol), NiCl2.dtbbpy (29.42 mg, 73.93 umol), Na₂CO₃ (3.13 g, 29.6 mmol) and DME (100 mL). The vial was sealed and placed with nitrogen gas. The reaction was stirred and irradiated with a 34 W blue LED lamp (7 cm away), with cooling fan to keep the reaction temperature at 25° C. for 15 hr. On completion, the reaction mixture was filtered and the cake was washed with DCM (30 mL), then the filtrate was concentrated to give an oil. The resulting oil was purified by silica gel column chromatography (PE:EA=100:1-10:1) to give the crude product (4.0 g). The crude product was triturated with EA (25 mL) at 20° C. for 30 min and then filtered to give the title compound (3.76 g, 34% yield) as yellow oil. ¹H NMR (400 MHz, CDCl₃) δ 8.20 (br s, 1H), 6.96-6.82 (m, 2H), 6.71 (d, J=7.8 Hz, 1H), 5.22 (dd, J=5.2, 12.6 Hz, 1H), 4.51 (br s, 1H), 3.43 (s, 3H), 3.10 (br d, J=6.0 Hz, 2H), 3.00-2.69 (m, 3H), 2.68-2.58 (m, 2H), 2.29-2.16 (m, 1H), 1.62-1.56 (m, 2H), 1.45 (s, 10H), 1.38-1.16 (m, 13H), LC-MS (ESI⁺) m/z 415.2 (M+1-100)⁺.

### Step 2—3-(5-(10-Aminodecyl)-3-methyl-2-oxo-2,3-dihydro-1H-benzo[d]imidazol-1-yl)piperidine-2,6-dione Trifluoroacetate

To a stirred mixture of tert-butyl N-{10-[1-(2,6-dioxopiperidin-3-yl)-3-methyl-2-oxo-1,3-benzodiazol-5-yl]decyl}carbamate (150 mg, 0.291 mmol) in DCM (3 mL) was added TFA (0.6 mL) dropwise at rt. The resulting mixture was stirred for 1 h at rt. On completion, the reaction mixture was concentrated under reduced pressure. The residue was triturated with diethyl ether (5 mL) to give the title compound (98 mg) as a white solid. LC/MS (ESI, m/z): [(M+H)]⁺=415.2.

### Example 1.6. Preparation of 6-chloro-8-{[(4-methoxyphenyl)methyl](methyl)amino}imidazo[1,2-b]pyridazine-3-carboxylic Acid (Intermediate F)

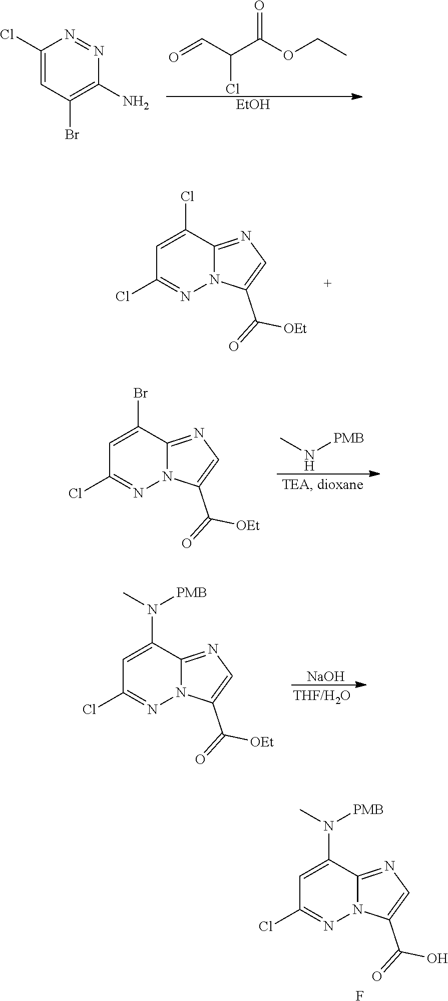

### Step 1—Ethyl 6,8-dichloroimidazo[1,2-b]pyridazine-3-carboxylate and ethyl 8-bromo-6-chloroimidazo[1,2-b]pyridazine-3-carboxylate

To a stirred solution of 4-bromo-6-chloropyridazin-3-amine (50 g, 240 mmol, CAS #446273-59-2) in EtOH (500 mL) was added ethyl 2-chloro-3-oxopropanoate (43.34 g, 287.9 mmol) at rt under nitrogen atmosphere. The resulting mixture was then stirred for 16 h at 90° C. under nitrogen atmosphere. On completion, the reaction mixture was concentrated under reduced pressure. The residue was triturated with Et₂O (500 mL) and stirred for 1 h, then filtered. The filter cake was washed with diethyl ether (3×500 mL) and dried in vacuo to afford mixture of ethyl 6,8-dichloroimidazo[1,2-b]pyridazine-3-carboxylate and ethyl 8-bromo-6-chloroimidazo[1,2-b]pyridazine-3-carboxylate (53 g) as a yellow solid. LC/MS (ESI, m/z): [(M+H)]⁺=260.0; 304.0, 306.0.

### Step 2—Ethyl 6-chloro-8-{[(4-methoxyphenyl)methyl](methyl)amino}imidazo[1,2-b]pyridazine-3-carboxylate

To a stirred solution of (mixture of ethyl 6,8-dichloroimidazo[1,2-b]pyridazine-3-carboxylate and ethyl 8-bromo-6-chloroimidazo[1,2-b]pyridazine-3-carboxylate (20 g, 66 mmol) and [(4-methoxyphenyl)methyl](methyl)amine (11.92 g, 78.81 mmol) in dioxane (500 mL) was added K₂CO₃ (18.15 g, 131.35 mmol) at rt under nitrogen atmosphere. The resulting mixture was stirred for 16 h at 90° C. under nitrogen atmosphere. On completion, the reaction mixture was cooled to rt and filtered. The filter cake was washed with 1,4-dioxane (3×10 mL) then the filtrate was concentrated under reduced pressure. The residue was triturated with Et₂O (200 mL) and stirred for 1 h, then filtered The filter cake was washed with diethyl ether (3×100 mL) and dried in vacuo to give the title compound (14 g, 57% yield) as a yellow solid. ¹H NMR (400 MHz, Chloroform-d) δ 8.10 (s, 1H), 7.17-7.13 (m, 2H), 6.87-6.83 (m, 2H), 6.10 (s, 1H), 5.48 (s, 2H), 4.46-4.40 (m, 2H), 3.79 (s, 3H), 3.16 (s, 3H), 1.43 (t, J=6.8 Hz, 3H). LC/MS (ESI, m/z): [(M+H)]⁺=375.1.

### Step 3—6-Chloro-8-{[(4-methoxyphenyl)methyl](methyl)amino}imidazo[1,2-b]pyridazine-3-carboxylic Acid

To a stirred solution of ethyl 6-chloro-8-{[(4-methoxyphenyl)methyl](methyl)amino}imidazo[1,2-b]pyridazine-3-carboxylate (13 g, 35 mmol) in THF (300 mL) was added aq. 1 N NaOH (300 mL) dropwise at rt. The resulting mixture was stirred for 16 h at 40° C. under nitrogen atmosphere. On completion, the reaction mixture was concentrated under reduced pressure. The residue was acidified to pH 5 with HCl (aq.). The precipitated solids were collected by filtration, washed with H₂O (2×500 mL), and dried in vacuo to afford the title compound (9.5 g, 79% yield) as a yellow solid. ¹H NMR (400 MHz, DMSO-d₆) δ 12.68 (s, 1H), 8.10 (s, 1H), 7.20-7.11 (m, 2H), 6.96-6.82 (m, 2H), 6.35 (s, 1H), 5.48 (s, 2H), 3.72 (s, 3H), 3.16 (s, 3H). LC/MS (ESI, m/z): [(M+H)]⁺=347.1.

### Example 1.7. Preparation of 6-chloro-N-[(1R,2S)-2-fluorocyclopropyl]-8-{[(4-methoxyphenyl)methyl](methyl)amino}imidazo[1,2-b]pyridazine-3-carboxamide (Intermediate G)

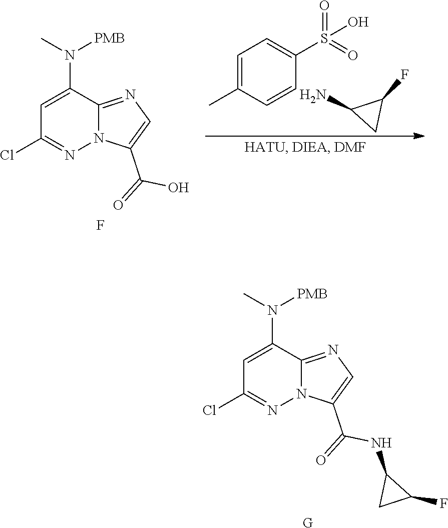

To a stirred solution of 6-chloro-8-{[(4-methoxyphenyl)methyl](methyl)amino}imidazo[1,2-b]pyridazine-3-carboxylic acid (9.5 g, 27 mmol, Intermediate F) and (1R,2S)-2-Fluorocyclopropanamine 4-methylbenzenesulfonate (8.06 g, 32.9 mmol, CAS #143062-84-4) in DMA (100 mL) were added HATU (12.50 g, 32.88 mmol) and DIEA (14.32 mL, 82.19 mmol) at rt under nitrogen atmosphere. The resulting mixture was stirred for 2 h at rt under nitrogen atmosphere. On completion, the reaction mixture was washed with H₂O (500 mL). The precipitated solids were collected by filtration and washed with H₂O (3×10 mL) to afford the title compound (9.4 g, 85% yield) as a yellow solid. ¹H NMR (400 MHz, DMSO-d₆) δ 8.77 (d, J=4.7 Hz, 1H), 8.11 (s, 1H), 7.25-7.17 (m, 2H), 6.93-6.82 (m, 2H), 6.39 (s, 1H), 5.51 (s, 2H), 5.03-4.83 (m, 1H), 3.72 (s, 3H), 3.20 (s, 3H), 3.08-2.94 (m, 1H), 1.28-1.19 (m, 1H), 1.03-0.93 (m, 1H). LC/MS (ESI, m/z): [(M+H)]⁺=404.1.

### Example 1.8. Preparation of methyl 3-amino-2-oxo-[1,2′-bipyridine]-5′-carboxylate (Intermediate H)

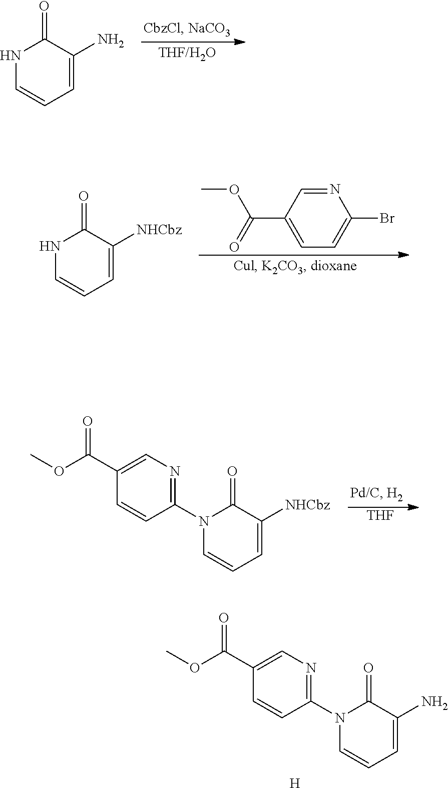

### Step 1—Benzyl N-(2-oxo-1H-pyridin-3-yl)carbamate

To a stirred solution of 3-amino-1H-pyridin-2-one (49 g, 440 mmol, CAS #33630-99-8) and Na₂CO₃ (70.74 g, 667.5 mmol) in THF (800 mL) and H₂O (800 mL) was added CbzCl (83.50 mL, 489.5 mmol) dropwise at 0° C. under nitrogen atmosphere. The resulting mixture was stirred 16 h at rt under nitrogen atmosphere. On completion, the reaction mixture was concentrated under reduced pressure. The residue was triturated with H₂O (2 L) and the mixture was filtered. The filter cake was washed with MeCN (2×50 mL). The filtrate was concentrated under reduced pressure to afford the title compound (40 g, 37% yield) as a purple solid. ¹H NMR (400 MHz, DMSO-d₆) δ 11.94 (s, 1H), 8.30 (s, 1H), 7.84 (dd, J=7.3, 1.8 Hz, 1H), 7.42-7.36 (m, 5H), 7.08 (dd, J=6.7, 1.9 Hz, 1H), 6.23 (t, J=6.9 Hz, 1H), 5.17 (s, 2H). LC/MS (ESI, m/z): [(M+H)]⁺=245.1.

### Step 2—Methyl 3-{[(benzyloxy)carbonyl]amino}-2-oxo-[1,2′-bipyridine]-5′-carboxylate

To a stirred solution of benzyl N-(2-oxo-1H-pyridin-3-yl)carbamate (20 g, 81.883 mmol) and methyl 6-bromopyridine-3-carboxylate (15.92 g, 73.695 mmol, CAS #26218-78-0) in 1,4-dioxane (800 mL) were added DMEDA (1.44 g, 16.38 mmol), CuI (3.12 g, 16.4 mmol) and K₂CO₃ (22.63 g, 163.8 mmol) at rt. The resulting mixture was stirred for additional 16 h at 100° C. On completion, the reaction mixture was filtered and the filter cake was washed with 1,4-dioxane (3×100 mL). The filtrate was concentrated under reduced pressure. The residue was purified by silica gel column chromatography, eluted with PE/EA (3:1), to afford the title compound (17 g, 55% yield) as a white solid. ¹H NMR (400 MHz, Chloroform-d) δ 9.18 (d, J=2.2 Hz, 1H), 8.45 (dd, J=8.5, 2.3 Hz, 1H), 8.13 (dd, J=8.3, 3.7 Hz, 2H), 7.95 (s, 1H), 7.69 (dd, J=7.3, 1.8 Hz, 1H), 7.46-7.33 (m, 5H), 6.41 (t, J=7.3 Hz, 1H), 5.18 (s, 2H), 4.01 (s, 3H). LC/MS (ESI, m/z): [(M+H)]⁺=380.1.

### Step 3—Methyl 3-amino-2-oxo-[1,2′-bipyridine]-5′-carboxylate

To a solution of methyl 3-{[(benzyloxy)carbonyl]amino}-2-oxo-[1,2′-bipyridine]-5′-carboxylate (10 g, 26 mmol) in THF (30 mL) was added Pd/C (4.21 g, 39.5 mmol) under argon atmosphere. The reaction system was degassed under vacuum and purged with H₂ several times. Then the mixture was hydrogenated under H₂ balloon (1 atm) at 25° C. for 4 h. On completion, Pd/C was filtered off through celite and the filter cake was washed with DCM (5×5 mL). The corresponding filtrate was concentrated under reduced pressure to provide the title compound (5.5 g, 85% yield) as a yellow solid. ¹H NMR (400 MHz, DMSO-d6) δ 9.08 (d, J=2.3 Hz, 1H), 8.45 (dd, J=8.5, 2.3 Hz, 1H), 8.04 (d, J=8.5 Hz, 1H), 7.23 (dd, J=7.1, 1.7 Hz, 1H), 6.53 (dd, J=7.1, 1.7 Hz, 1H), 6.23 (t, J=7.1 Hz, 1H), 5.34 (s, 2H). 3.93 (s, 3H). LC/MS (ESI, m/z): [(M+H)]⁺=246.1.

### Example 1.9. Preparation of 33-((3-(((1R,2S)-2-fluorocyclopropyl)carbamoyl)-8-(methylamino)imidazo[1,2-b]pyridazin-6-yl)amino)-2-oxo-2H-[1,2′-bipyridine]-5′-carboxylic Acid (Intermediate I)

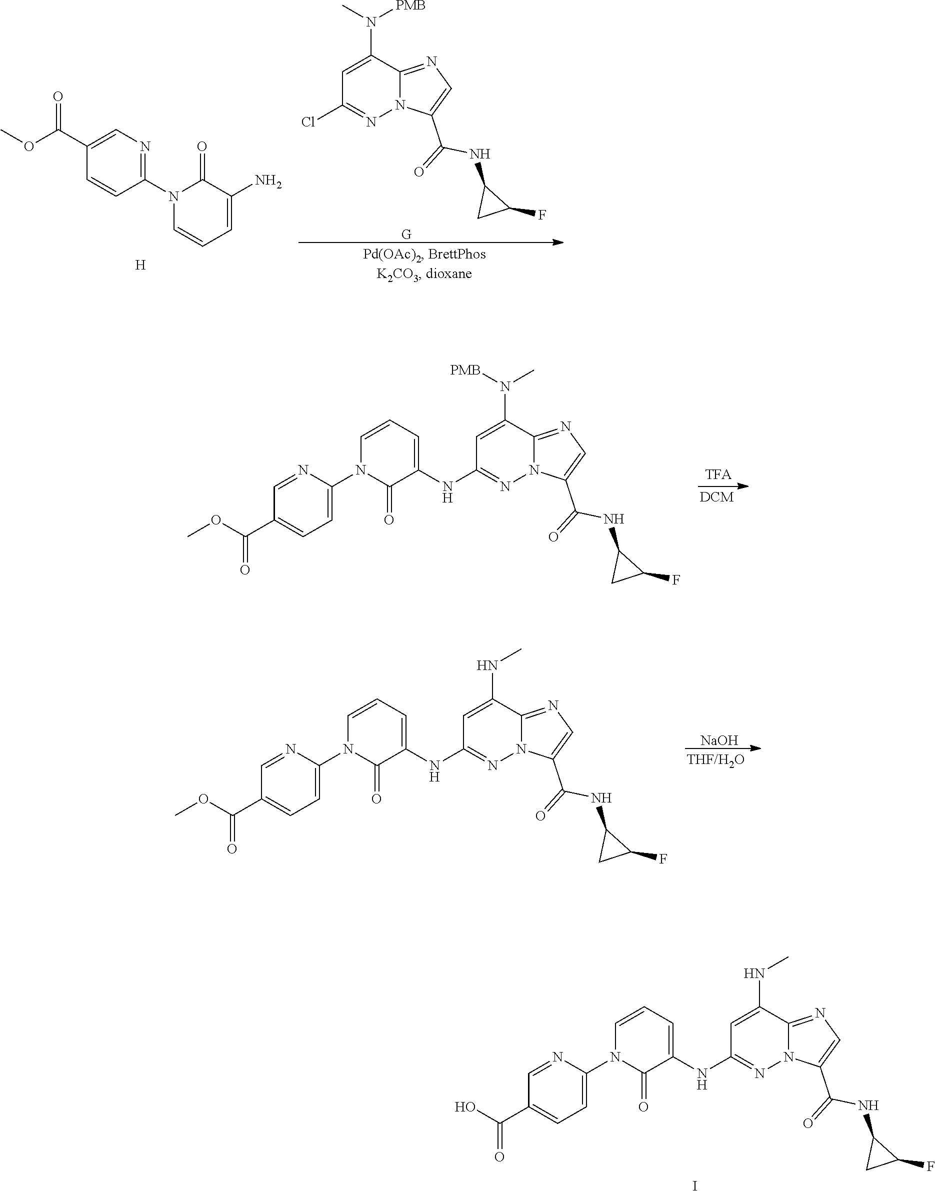

### Step 1—Methyl 3-[(3-{[(1R,2S)-2-fluorocyclopropyl]carbamoyl}-8-{[(4-methoxyphenyl)methyl](methyl)amino}imidazo[1,2-b]pyridazin-6-yl)amino]-2-oxo-[1,2′-bipyridine]-5′-carboxylate

To a stirred mixture of 6-chloro-N-[(1R,2S)-2-fluorocyclopropyl]-8-{[(4-methoxyphenyl)methyl](methyl)amino}imidazo[1,2-b]pyridazine-3-carboxamide (1.9 g, 4.7 mmol, Intermediate G) and methyl 3-amino-2-oxo-[1,2′-bipyridine]-5′-carboxylate (1.73 g, 7.06 mmol, Intermediate H) in 1,4-dioxane (90 mL) were added K₂CO₃ (1.95 g, 14.1 mmol), Pd(OAc)₂ (0.11 g, 0.47 mmol) and Brettphos (0.25 g, 0.47 mmol) at rt under argon atmosphere. The resulting mixture was then stirred for 2 h at 100° C. On completion, the reaction mixture was allowed to cool down to rt and the resulting mixture was filtered. The filter cake was washed with DCM then the filtrate was concentrated under reduced pressure. The residue was purified by reverse phase flash chromatography (Column: WelFlash™ C18-I, 20-40 um, 330 g; Eluent A: Water (plus 10 mmol/L NH₄HCO₃); Eluent B: ACN; Gradient: 50%-70% B in 25 min; Flow rate: 80 mL/min; Detector: 220/254 nm; desired fractions were collected at 66% B) and concentrated under reduced pressure to afford the title compound (2.38 g, 53% yield) as a yellow solid. ¹H NMR (400 MHz, Chloroform-d) δ 9.22 (d, J=2.3 Hz, 1H), 8.80 (d, J=4.5 Hz, 1H), 8.49 (dd, J=8.6, 2.3 Hz, 1H), 8.23 (s, 1H), 8.16 (d, J=8.5 Hz, 1H), 7.87 (dd, J=7.4, 1.6 Hz, 1H), 7.73-7.62 (m, 2H), 7.22-7.13 (m, 2H), 6.91-6.83 (m, 2H), 6.46 (t, J=7.3 Hz, 1H), 5.68 (s, 1H), 5.44 (s, 2H), 4.89-4.73 (m, 1H), 4.02 (s, 3H), 3.81 (s, 3H), 3.22 (s, 3H), 3.31-3.15 (m, 1H), 1.34-1.29 (m, 1H), 1.18-1.08 (m, 1H). LC/MS (ESI, m/z): [(M+H)]⁺=613.3.

### Step 2—Methyl 3-[(3-{[(1R,2S)-2-fluorocyclopropyl]carbamoyl}-8-(methylamino)imidazo[1,2-b]pyridazin-6-yl)amino]-2-oxo-[1,2′-bipyridine]-5′-carboxylate

To a stirred solution methyl 3-[(3-{[(1R,2S)-2-fluorocyclopropyl]carbamoyl}-8-{[(4-methoxyphenyl)methyl](methyl)amino}imidazo[1,2-b]pyridazin-6-yl)amino]-2-oxo-[1,2′-bipyridine]-5′-carboxylate (2.38 g, 3.89 mmol) in DCM (25 mL) was added TFA (5 mL) dropwise at rt. The resulting mixture was stirred for 1 h at rt under nitrogen atmosphere. On completion, the reaction mixture was concentrated under reduced pressure. The residue was triturated with diethyl ether (20 mL). The precipitated solids were collected by filtration and washed with diethyl ether (3×8 mL), then dried in vacuo to provide the title compound (2.0 g) as an off-white solid. LC/MS (ESI, m/z): [(M+H)]⁺=493.2.

### Step 3—3-[(3-{[(1R,2S)-2-fluorocyclopropyl]carbamoyl}-8-(methylamino)imidazo[1,2-b]pyridazin-6-yl)amino]-2-oxo-[1,2′-bipyridine]-5′-carboxylic Acid

To a stirred solution of methyl 3-[(3-{[(1R,2S)-2-fluorocyclopropyl]carbamoyl}-8-(methylamino)imidazo[1,2-b]pyridazin-6-yl)amino]-2-oxo-[1,2′-bipyridine]-5′-carboxylate (2 g, 4 mmol) in THF (20 mL) was added 1 N aq. NaOH (20 mL) dropwise at rt. The resulting mixture was stirred for 0.5 h at rt under nitrogen atmosphere. On completion, the reaction was acidified by 1 N aq. HCl to pH=3. The precipitated solids were collected by filtration and washed with water (5×5 mL). The solid was dried in vacuo to give the title compound (1.1 g, 57% yield) as a white solid. ¹H NMR (400 MHz, DMSO-d₆) δ 13.54 (br, s, 1H), 9.10 (dd, J=2.3, 0.8 Hz, 1H), 8.67 (s, 1H), 8.64 (d, J=4.2 Hz, 1H), 8.47 (dd, J=8.4, 2.3 Hz, 1H), 8.03 (dd, J=8.4, 0.8 Hz, 1H), 8.00 (dd, J=7.4, 1.8 Hz, 1H), 7.92 (s, 1H), 7.62 (dd, J=7.1, 1.7 Hz, 1H), 7.50 (q, J=4.9 Hz, 1H), 6.44 (t, J=7.2 Hz, 1H), 6.40 (s, 1H), 4.98-4.78 (m, 1H), 3.08-2.97 (m, 1H), 2.88 (d, J=4.4 Hz, 3H), 1.29-1.19 (m, 1H), 1.05-0.94 (m, 1H).

### Example 1.10. Preparation of 4,6-dichloro-N-methylnicotinamide (Intermediate J)

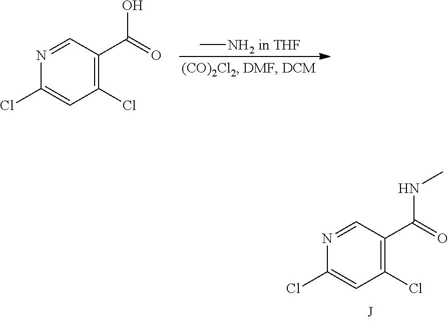

To a stirred solution of 4,6-dichloropyridine-3-carboxylic acid (100 g, 500 mmol, CAS #73027-79-9) and (COCl)₂ (66.55 mL, 781.3 mmol) in DCM (500 mL) was added DMF (4.03 mL, 52.1 mmol) dropwise at 0° C. under nitrogen atmosphere. The resulting mixture was stirred for 1 h at rt under nitrogen atmosphere. On completion, the reaction mixture was concentrated under reduced pressure. The residue was re-dissolved in DCM (500 mL) and marked solution A. To a solution of methylamine (520.83 mL, 1041.7 mmol) in THF was added solution A dropwise over 15 min at 0° C. The resulting mixture was stirred for additional 2 h at rt. On completion, the reaction mixture was concentrated under reduced pressure. The residue was purified by silica gel column chromatography, eluted with PE/EA (1:1), to afford the title compound (87 g, 82% yield) as a white solid. ¹H NMR (400 MHz, Chloroform-d) δ 8.61 (s, 1H), 7.28 (s, 1H), 6.43 (s, 1H), 3.04 (d, J=4.9 Hz, 3H). LC/MS (ESI, m/z): [(M+H)]⁺=205.0.

### Example 1.11. Preparation of 6-chloro-4-((3-(5-fluoropyrimidin-2-yl)-2-methoxyphenyl)amino)-N-methylnicotinamide (Intermediate K)

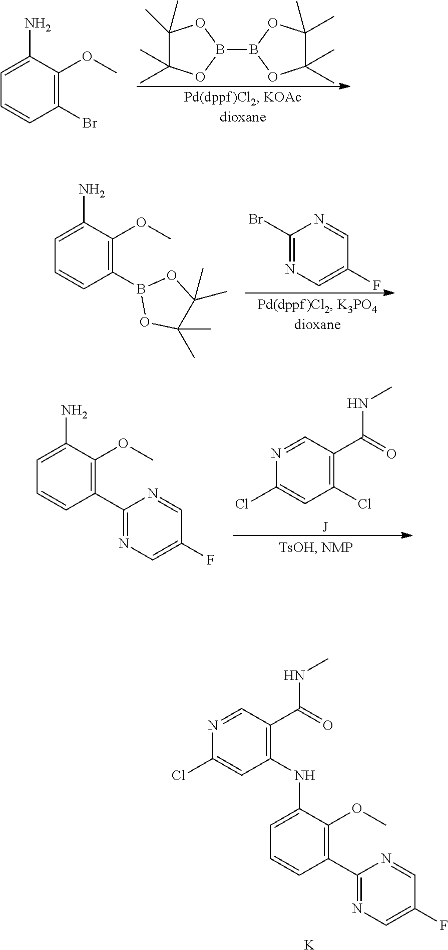

### Step 1—2-Methoxy-3-(4,4,5,5-tetramethyl-1,3,2-dioxaborolan-2-yl)aniline

To a stirred solution of 3-bromo-2-methoxyaniline (50 g, 250 mmol) and bis(pinacolato)diboron (69.12 g, 272.2 mmol) in dioxane (500 mL) were added Pd(dppf)Cl₂ (18.11 g, 24.75 mmol) and KOAc (48.57 g, 494.9 mmol) in portions at rt under nitrogen atmosphere. The resulting mixture was stirred for 16 h at 100° C. under N₂ atmosphere. On completion, the reaction mixture was cooled to rt and filtered, then the filter cake was washed with DCM (3×100 mL). The filtrate was concentrated under reduced pressure. The residue was purified by silica gel column chromatography, eluted with PE/EA (1:1) to afford the title compound (37 g, 60% yield) as a brown yellow oil. ¹H NMR (400 MHz, Chloroform-d) δ 7.14 (dd, J=7.2, 1.8 Hz, 1H), 6.95 (t, J=7.5 Hz, 1H), 6.89 (dd, J=7.8, 1.8 Hz, 1H), 3.98 (br, s, 2H), 3.84 (s, 3H), 1.38 (s, 12H). LC/MS (ESI, m/z): [(M+H)]⁺=250.2.

### Step 2—3-(5-Fluoropyrimidin-2-yl)-2-methoxyaniline

To a stirred solution of 2-methoxy-3-(4,4,5,5-tetramethyl-1,3,2-dioxaborolan-2-yl)aniline (10 g, 40 mmol) and 2-bromo-5-fluoropyrimidine (10.66 g, 60.21 mmol) in dioxane (200 mL) was added H₂O (40 mL), K₃PO₄ (17.04 g, 80.28 mmol) and Pd(dppf)Cl₂ (2.94 g, 4.01 mmol) in turns at rt under nitrogen atmosphere. The resulting mixture was stirred for 16 h at 100° C. under nitrogen atmosphere. On completion, the reaction mixture was allowed to cooled to rt and filtered. The filter cake was washed with DCM (3×100 mL) and the filtrate was concentrated under reduced pressure. The residue was purified by silica gel column chromatography, eluted with PE/EA (1:1), to afford the title compound (7.5 g, 85% yield) as a brown yellow oil. ¹H NMR (400 MHz, Chloroform-d) δ 8.74 (s, 2H), 7.2-7.17 (m, 1H), 7.09-7.00 (m, 1H), 6.96-6.85 (m, 1H), 4.18 (br, s, 2H), 3.69 (s, 3H); LC/MS (ESI, m/z): [(M+H)]⁺=220.1.

### Step 3—6-Chloro-4-((3-(5-fluoropyrimidin-2-yl)-2-methoxyphenyl)amino)-N-methylnicotinamide

To a stirred solution of 3-(5-fluoropyrimidin-2-yl)-2-methoxyaniline (5 g, 20 mmol) and 4,6-dichloro-N-methylpyridine-3-carboxamide (4.68 g, 22.8 mmol, Intermediate J) in NMP (75 mL) was added TsOH (5.89 g, 34.2 mmol) at rt under N₂ atmosphere. The resulting mixture was stirred for 16 h at 100° C. under nitrogen atmosphere. On completion, the reaction mixture was cooled to rt. The reaction mixture was quenched by sat.aq. NaHCO₃ (100 mL) and the resulting mixture was extracted with EtOAc (3×100 mL). The combined organic layers were washed with brine (3×50 mL), dried over anhydrous Na₂SO₄, and filtered. The filtrate was concentrated under reduced pressure. The residue was purified by silica gel column chromatography, eluted with PE/EA (1:1) to afford the title compound (3 g, 34% yield) as a light yellow solid. ¹H NMR (400 MHz, Chloroform-d) δ 10.27 (s, 1H), 8.75 (d, J=2.3 Hz, 2H), 8.40 (d, J=2.2 Hz, 1H), 7.70-7.63 (m, 1H), 7.51-7.44 (m, 1H), 7.32-7.23 (m, 1H), 6.98 (d, J=2.2 Hz, 1H), 6.75 (s, 1H), 3.78 (d, J=2.2 Hz, 3H), 3.08-3.01 (m, 3H). LC/MS (ESI, m/z): [(M+H)]⁺=388.1.

### Example 1.12. Preparation of 6-amino-4-((3-(5-fluoropyrimidin-2-yl)-2-methoxyphenyl)amino)-N-methylnicotinamide (Intermediate L)

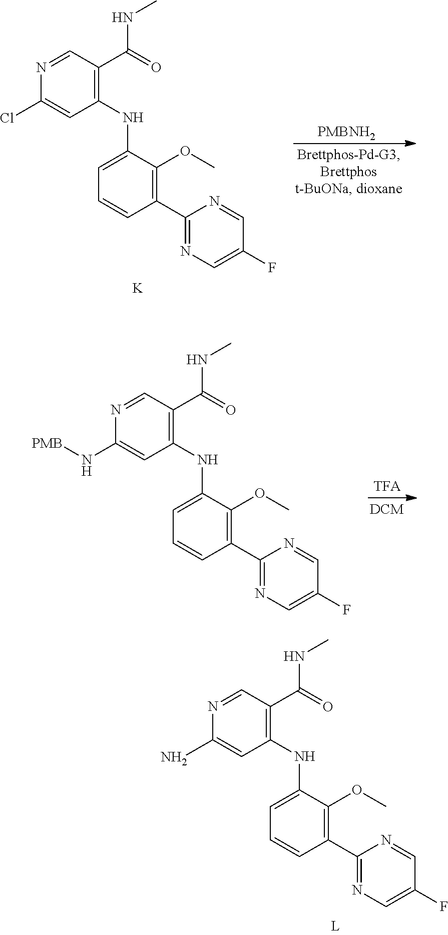

### Step 1—4-((3-(5-fluoropyrimidin-2-yl)-2-methoxyphenyl)amino)-6-((4-methoxybenzyl)amino)-N-methylnicotinamide

To a stirred solution of 6-chloro-4-{[3-(5-fluoropyrimidin-2-yl)-2-methoxyphenyl]amino}-N-methylpyridine-3-carboxamide (7 g, 20 mmol, Intermediate K) and benzenemethanamine, 4-methoxy- (2.97 g, 21.7 mmol) in dioxane (100 mL) were added BrettPhos (0.97 g, 1.8 mmol), t-BuONa (3.47 g, 36.1 mmol) and BrettPhos Pd G3 (1.64 g, 1.80 mmol) in turns at rt under nitrogen atmosphere. The resulting mixture was stirred for 2 h at 100° C. under N₂ atmosphere. On completion, the reaction mixture was allowed to cool to rt and quenched by sat.aq. NaHCO₃ (50 mL). The resulting mixture was extracted with EtOAc (3×50 mL). The combined organic layers were washed with water (3×50 mL), dried over anhydrous Na₂SO₄. After filtration, the filtrate was concentrated under reduced pressure. The residue was purified by silica gel column chromatography, eluted with PE/EA (1:1.5) to afford the title compound (5 g, 57% yield) as a white solid. ¹H NMR (400 MHz, Chloroform-d) δ 10.17 (s, 1H), 8.73 (s, 2H), 8.15 (s, 1H), 7.56-7.43 (m, 1H), 7.25-7.20 (m, 4H), 7.08 (d, J=7.9 Hz, 1H), 6.90-6.85 (m, 2H), 6.14 (s, 1H), 6.02 (s, 1H), 4.32 (d, J=5.3 Hz, 2H), 3.81 (s, 3H), 3.78 (s, 3H), 2.97 (d, J=4.8 Hz, 3H). LC/MS (ESI, m/z): [(M+H)]⁺=489.2.

### Step 2—6-amino-4-((3-(5-fluoropyrimidin-2-yl)-2-methoxyphenyl)amino)-N-methylnicotinamide

To a stirred solution of 4-{[3-(5-fluoropyrimidin-2-yl)-2-methoxyphenyl]amino}-6-{[(4-methoxyphenyl)methyl]amino}-N-methylpyridine-3-carboxamide (5 g, 10 mmol) in DCM (40 mL) was added TFA (15 mL) dropwise at 0° C. under nitrogen atmosphere. The resulting mixture was stirred for 16 h at rt under air atmosphere. On completion, the reaction mixture was concentrated under reduced pressure. The residue was purified by reverse phase Flash chromatography (Column: WelFlash™ C18-I, 20-40 um, 120 g; Eluent A: Water (plus 10 mmol/L NH₄HCO₃); Eluent B: ACN; Gradient: 25%-55% B in 25 min; Flow rate: 60 mL/min; Detector: 220/254 nm; desired fractions were collected at 50% B) and concentrated under reduced pressure to afford the title compound (1.8 g, 48% yield) as a white solid. ¹H NMR (400 MHz, Chloroform-d) δ 10.11 (s, 1H), 8.74 (s, 2H), 8.18 (s, 1H), 7.59-7.52 (m, 1H), 7.51-7.44 (m, 1H), 7.26-7.17 (m, 1H), 6.28-6.22 (m, 1H), 6.11 (s, 1H), 4.56 (s, 2H), 3.79 (s, 3H), 2.99 (s, 3H). LC/MS (ESI, m/z): [(M+H)]⁺=369.3.

### Example 1.13. Preparation of 6-((4-((3-(5-fluoropyrimidin-2-yl)-2-methoxyphenyl)amino)-5-(methylcarbamoyl)pyridin-2-yl)amino)nicotinic Acid (Intermediate M)

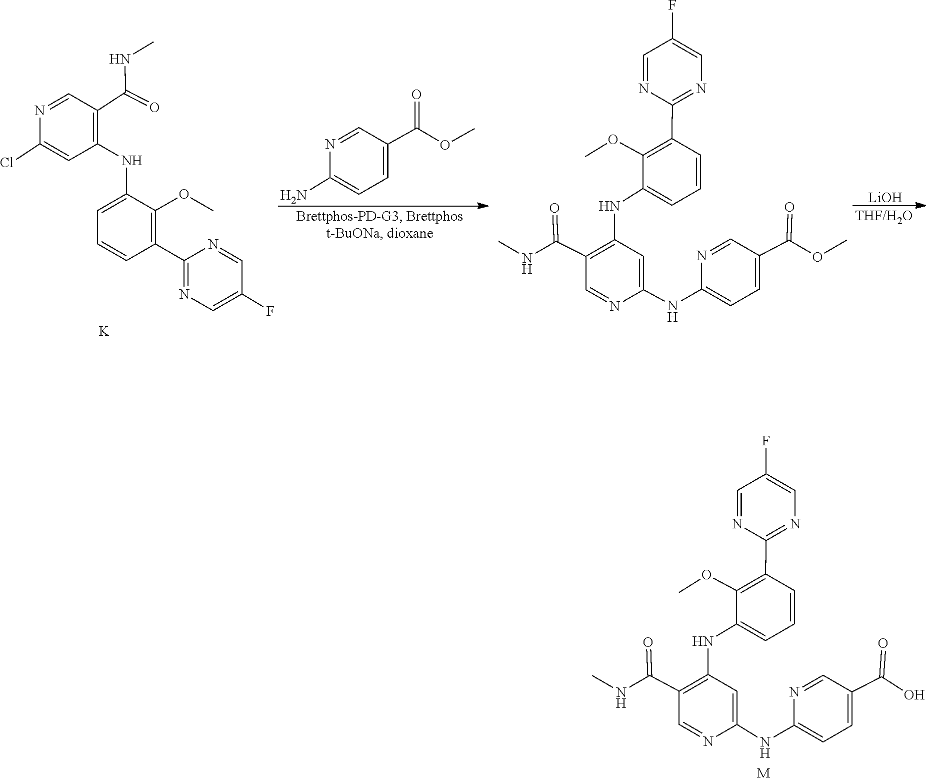

### Step 1—Methyl 6-((4-((3-(5-fluoropyrimidin-2-yl)-2-methoxyphenyl)amino)-5-(methylcarbamoyl)pyridin-2-yl)amino)nicotinate

To a stirred mixture of 6-chloro-4-{[3-(5-fluoropyrimidin-2-yl)-2-methoxyphenyl]amino}-N-methylpyridine-3-carboxamide (1 g, 3 mmol, Intermediate K) and methyl 6-aminopyridine-3-carboxylate (470.82 mg, 3.095 mmol) in dioxane were added BrettPhos Pd G3 (233.75 mg, 0.258 mmol), BrettPhos (138.42 mg, 0.258 mmol) and t-BuONa (495.64 mg, 5.158 mmol) in turns at rt under nitrogen atmosphere. The resulting mixture was stirred for 1 h at 120° C. under nitrogen atmosphere. On completion, the reaction mixture was allowed to cooled to rt and concentrated under reduced pressure. The residue was purified by reverse phase flash (Column: WelFlash™ C18-I, 20-40 um, 80 g; Eluent A: Water (10 mmol/L NH₄HCO₃); Eluent B: ACN; Gradient: 25%-55% B in 25 min; Flow rate: 60 mL/min; Detector: 254 nm; desired fractions were collected at 50% B) and concentrated under reduced pressure to afford the title compound (700 mg, 54% yield) as a white solid. ¹H NMR (400 MHz, DMSO-d6) δ 10.69 (s, 1H), 10.28 (s, 1H), 9.05 (d, J=0.8 Hz, 2H), 8.72 (d, J=2.3 Hz, 1H), 8.61-8.52 (m, 2H), 8.16-8.09 (m, 1H), 7.90 (s, 1H), 7.77-7.67 (m, 2H), 7.49-7.42 (m, 1H), 7.38-7.30 (m, 1H), 3.84 (s, 3H), 3.70 (s, 3H), 2.81 (d, J=4.4 Hz, 3H). [(M+H)]⁺=504.1.

### Step 2—6-((4-((3-(5-fluoropyrimidin-2-yl)-2-methoxyphenyl)amino)-5-(methylcarbamoyl)pyridin-2-yl)amino)nicotinic Acid

To a stirred solution of methyl 6-[(4-{[3-(5-fluoropyrimidin-2-yl)-2-methoxyphenyl]amino}-5-(methylcarbamoyl)pyridin-2-yl)amino]pyridine-3-carboxylate (430 mg, 0.854 mmol) in THF (5 mL) and water (5 mL) was added LiOH (204.54 mg, 8.540 mmol) at rt under nitrogen atmosphere. The resulting mixture was stirred for 2 h at rt under nitrogen atmosphere. On completion, the reaction mixture was concentrated under reduced pressure. The mixture was acidified to pH 3 with HCl (aq. 2 mol/L). The precipitated solids were collected by filtration and washed with water, then dried under vacuum to provide the title compound (407 mg, 97% yield) as a light yellow solid. ¹H NMR (400 MHz, DMSO-d₆) δ 13.41 (s, 1H), 11.87 (s, 1H), 10.95 (s, 1H), 9.19 (s, 1H), 9.06 (s, 2H), 8.83 (d, J=2.3 Hz, 1H), 8.60 (s, 1H), 8.25 (dd, J=8.7, 2.3 Hz, 1H), 7.73-7.57 (m, 2H), 7.38 (t, J=7.9 Hz, 1H), 7.34-7.25 (m, 1H), 7.11 (s, 1H), 3.71 (s, 3H), 2.83 (d, J=4.4 Hz, 3H). LC/MS (ESI, m/z): [(M+H)]⁺=490.1.

### Example 1.14. Preparation of 3-[4-(4-aminobutyl)-3-methyl-2-oxo-1,3-benzodiazol-1-yl]piperidine-2,6-dione (Intermediate N)

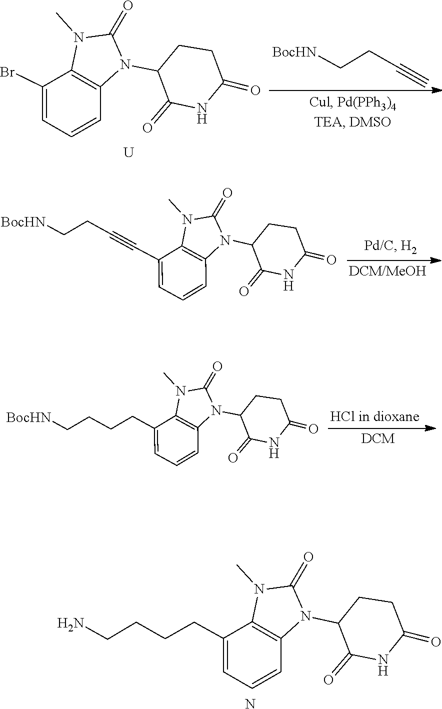

### Step 1—Tert-butyl N-[4-[1-(2,6-dioxopiperidin-3-yl)-3-methyl-2-oxo-1,3-benzodiazol-4-yl]but-3-yn-1-yl]carbamate

To a stirred solution of 3-(4-bromo-3-methyl-2-oxo-1,3-benzodiazol-1-yl)piperidine-2,6-dione (8.00 g, 23.7 mmol, Intermediate U, CAS #2304754-51-4) in DMA (40.00 mL) and TEA (20.00 mL) were added tert-butyl N-(but-3-yn-1-yl)carbamate (12.01 g, 70.97 mmol, CAS #149990-27-2), CuI (450.55 mg, 2.366 mmol) and Pd(PPh₃)₄ (2733.75 mg, 2.366 mmol) at rt. The resulting mixture was stirred at 85° C. for 3 h under nitrogen atmosphere. The reaction was acidified pH to 6 with FA. Then reaction mixture was diluted with EA (500 mL), washed with brine (2×200 mL), then the organic layers was dried over anhydrous Na₂SO₄. After filtration, the filtrate was concentrated under reduced pressure. The residue was purified by silica gel column chromatography, eluted with DCM:MeOH (10:1), to afford the title compound (5.8 g, 58% yield) as a white solid. LC/MS (ESI, m/z): [(M+1)]⁺=427.2.

### Step 2—Tert-butyl N-[4-[1-(2,6-dioxopiperidin-3-yl)-3-methyl-2-oxo-1,3-benzodiazol-4-yl]butyl]carbamate

To a stirred mixture of tert-butyl N-[4-[1-(2,6-dioxopiperidin-3-yl)-3-methyl-2-oxo-1,3-benzodiazol-4-yl]but-3-yn-1-yl]carbamate (5.80 g, 13.6 mmol) in MeOH (50.00 mL) and DCM (500.00 mL) was added Pd/C (1.45 g, 13.6 mmol, 10 wt %) at rt under nitrogen atmosphere. The mixture was stirred for 4 h under hydrogen atmosphere at rt. On completion, the reaction mixture was filtered and the filter cake was washed with MeOH (3×100 mL). The filtrate was concentrated under reduced pressure to give the title compound (5 g, 85% yield) as a white solid. LC/MS (ESI, m/z): [(M+1)]⁺=431.2.

### Step 3—3-[4-(4-Aminobutyl)-3-methyl-2-oxo-1,3-benzodiazol-1-yl]piperidine-2,6-dione

To a stirred solution of tert-butyl N-[4-[1-(2,6-dioxopiperidin-3-yl)-3-methyl-2-oxo-1,3-benzodiazol-4-yl]butyl]carbamate (5.00 g, 11.6 mmol) in DCM (40.00 mL) was added HCl (4M) in 1,4-dioxane (40.00 mL) at rt under nitrogen atmosphere. The solution was stirred for 4 h at rt. On completion, the reaction mixture was concentrated under reduced pressure. The residue product was purified by reverse phase flash (Column: Spherical C18 Column, 20-40 um, 330 g; Mobile Phase A: Water (5 mmol/L NH₄HCO₃), Mobile Phase B: ACN; Flow rate: 100 mL/min; Gradient: 25% B to 45% B in 25 min, 254 nm; the fractions containing the desired product were collected at 33% B) to afford the title compound (3.8 g, 99% yield) as a white solid. LC/MS (ESI, m/z): [(M+1)]⁺=331.2.

### Example 1.15. Preparation of methyl 3-amino-2-oxo-[1,2′-bipyridine]-4′-carboxylate (Intermediate O)

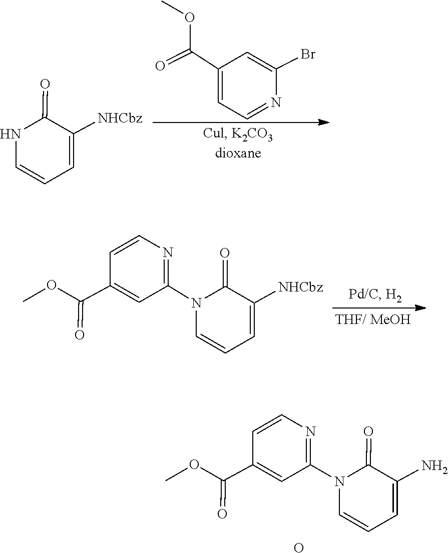 [SMILES: O=C(Nc1ccc[nH]c1=O)OCc1ccccc1 ; COC(=O)c1ccnc(Br)c1 ; COC(=O)c1ccnc(-n2cccc(NC(=O)OCc3ccccc3)c2=O)c1 ; COC(=O)c1ccnc(-n2cccc(N)c2=O)c1 ; O]

### Step 1—Methyl 3-{[(benzyloxy)carbonyl]amino}-2-oxo-[1,2′-bipyridine]-4′-carboxylate

To a stirred solution of benzyl N-(2-oxo-1H-pyridin-3-yl)carbamate (10 g, 40 mmol, CAS #147269-67-8), methyl 2-bromopyridine-4-carboxylate (8.84 g, 40.9 mmol) and K₂CO₃ (11.32 g, 81.89 mmol) in dioxane (150 mL) were added DMEDA (360.91 mg, 4.094 mmol) and CuI (779.73 mg, 4.094 mmol) in portions at room temperature under N₂ atmosphere. The resulting mixture was stirred for 2 h at 110° C. under nitrogen atmosphere. On completion, the reaction mixture was cooled to rt and the resulting mixture was concentrated under vacuum. The residue was purified by silica gel column chromatography, eluted with PE/EA (5:1), to afford the title compound (8.29 g, 53% yield) as a white solid. ¹H NMR (400 MHz, Chloroform-d) δ 8.73 (dd, J=5.0, 0.9 Hz, 1H), 8.52-8.48 (m, 1H), 8.13 (d, J=7.3 Hz, 1H), 7.97 (s, 1H), 7.92 (dd, J=5.1, 1.4 Hz, 1H), 7.60 (dd, J=7.2, 1.7 Hz, 1H), 7.47-7.33 (m, 5H), 6.41 (t, J=7.3 Hz, 1H), 5.25 (s, 2H), 4.00 (s, 3H). LC/MS (ESI, m/z): [(M+H)]⁺=380.2.

### Step 2—Methyl 3-amino-2-oxo-[1,2′-bipyridine]-4′-carboxylate

To a solution of methyl 3-{[(benzyloxy)carbonyl]amino}-2-oxo-[1,2′-bipyridine]-4′-carboxylate (8 g, 20 mmol) in THF (10 mL) and MeOH (10 mL) was added Pd/C (224.41 mg, 2.109 mmol) in a pressure tank. The mixture was hydrogenated at rt under 30 psi of hydrogen pressure for 2 h. On completion, the reaction mixture was filtered through a Celite pad. The filtrate was concentrated under reduced pressure. The residue was purified by silica gel column chromatography, eluted with PE/EA (5:1), to afford the title compound (4.7 g, 91% yield) as a yellow solid. ¹H NMR (400 MHz, DMSO-d₆) δ 8.79 (dd, J=5.0, 0.8 Hz, 1H), 8.29 (dd, J=1.5, 0.8 Hz, 1H), 7.89 (dd, J=5.0, 1.5 Hz, 1H), 7.22 (dd, J=7.0, 1.7 Hz, 1H), 6.54 (dd, J=7.1, 1.7 Hz, 1H), 6.23 (t, J=7.1 Hz, 1H), 5.32 (s, 3H), 3.94 (s, 3H). LC/MS (ESI, m/z): [(M+H)]⁺=246.1.

### Example 1.16. Preparation of 3-[(3-{[(1R,2S)-2-fluorocyclopropyl]carbamoyl}-8-(methylamino)imidazo[1,2-b]pyridazin-6-yl)amino]-2-oxo-[1,2′-bipyridine]-4′-carboxylic Acid (Intermediate P)

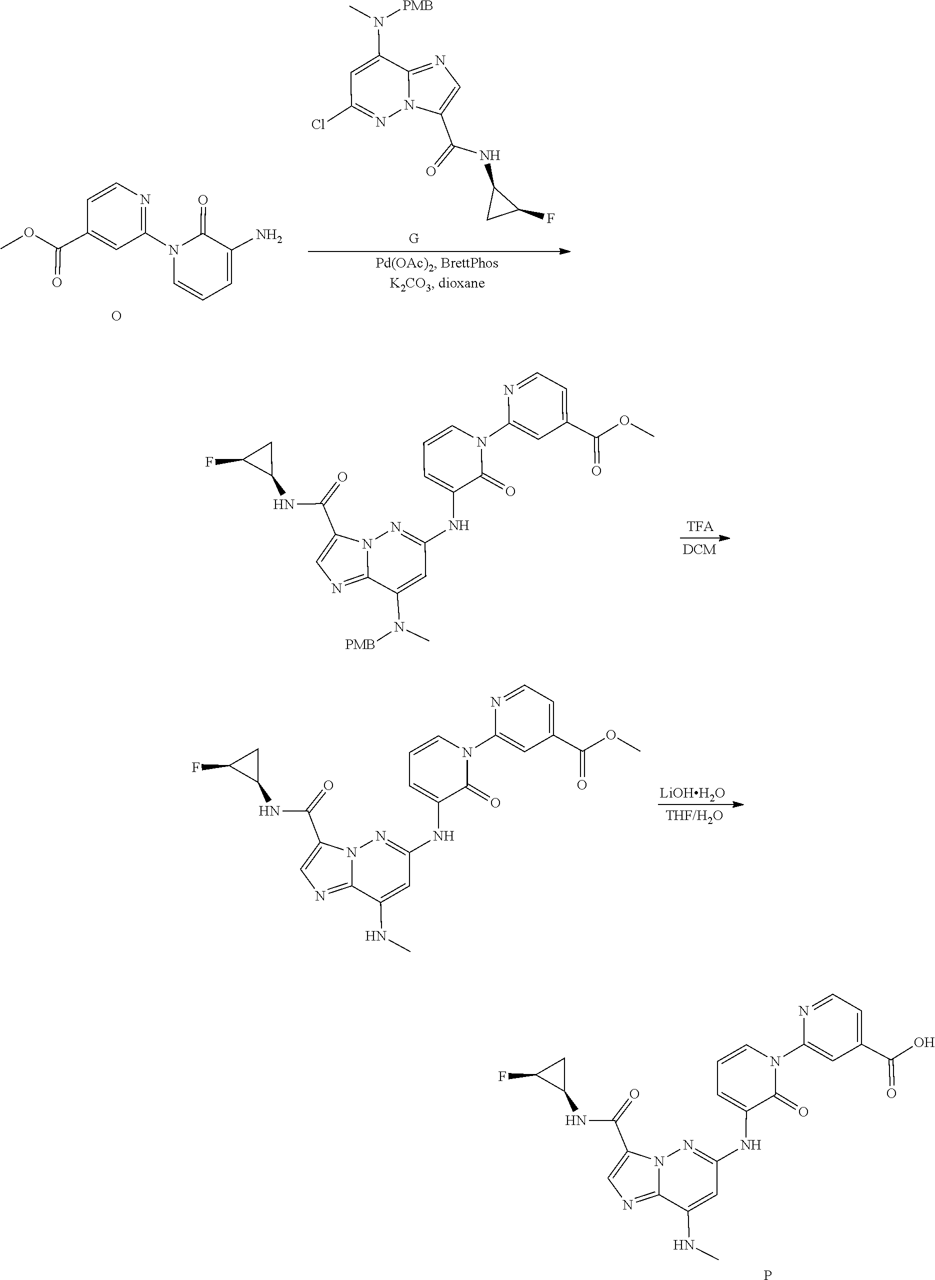

### Step 1—Methyl 3-[(3-{[(1R,2S)-2-fluorocyclopropyl]carbamoyl}-8-{[(4-methoxyphenyl)methyl](methyl)amino}imidazo[1,2-b]pyridazin-6-yl)amino]-2-oxo-[1,2′-bipyridine]-4′-carboxylate

To a stirred solution of methyl 3-amino-2-oxo-[1,2′-bipyridine]-4′-carboxylate (3.00 g, 12.2 mmol, Intermediate O) and 6-chloro-N-[(1R,2S)-2-fluorocyclopropyl]-8-{[(4-methoxyphenyl)methyl](methyl)amino}imidazo[1,2-b]pyridazine-3-carboxamide (4.11 g, 10.2 mmol, Intermediate G) in dioxane (120 mL) were added Pd(OAc)₂ (228.49 mg, 1.018 mmol), K₂CO₃ (2.81 g, 20.4 mmol), and BrettPhos (546.28 mg, 1.018 mmol) dropwise at rt under N₂ atmosphere. The resulting mixture was stirred for 2 h at 100° C. under N₂ atmosphere. On completion, the reaction mixture was concentrated under vacuum. The residue was purified by silica gel column chromatography, eluted with PE/EA (5:1), to afford the title compound (3.6 g, 58% yield) as a yellow solid. ¹H NMR (400 MHz, Chloroform-d) δ 8.79 (d, J=4.7 Hz, 1H), 8.77 (d, J=5.1 Hz, 1H), 8.56-8.53 (m, 1H), 8.25 (s, 1H), 7.95 (dd, J=5.1, 1.4 Hz, 1H), 7.88 (dd, J=7.3, 1.7 Hz, 1H), 7.74-7.70 (s, 1H), 7.60-7.56 (m, 1H), 7.22-7.16 (m, 2H), 6.90-6.86 (m, 2H), 6.46 (t, J=7.3 Hz, 1H), 5.69 (s, 1H), 5.41 (s, 2H), 4.91-4.71 (m, 1H), 4.01 (s, 3H), 3.81 (s, 3H), 3.24 (s, 3H), 3.21-3.18 (m, 1H), 1.36-1.29 (m, 1), 1.19-1.09 (m, 1H), LC/MS (ESI, m/z): [(M+H)]⁺=613.3.

### Step 2—Methyl 3-[(3-{[(1R,2S)-2-fluorocyclopropyl]carbamoyl}-8-(methylamino)imidazo[1,2-b]pyridazin-6-yl)amino]-2-oxo-[1,2′-bipyridine]-4′-carboxylate

A solution of methyl 3-[(3-{[(1R,2S)-2-fluorocyclopropyl]carbamoyl}-8-{[(4-methoxyphenyl)methyl](methyl)amino}imidazo[1,2-b]pyridazin-6-yl)amino]-2-oxo-[1,2′-bipyridine]-4′-carboxylate (3.62 g, 5.91 mmol) in DCM (45 mL) and TFA (15 mL) was stirred for 1 h at rt. On completion, the reaction mixture was concentrated under vacuum. The residue was purified by silica gel column chromatography, eluted with CHCl₃/MeOH (10:1), to afford the title compound (2.9 g, 99% yield) as a yellow solid. ¹H NMR (400 MHz, Chloroform-d) δ 8.78 (d, J=5.1 Hz, 1H), 8.60 (d, J=4.3 Hz, 1H), 8.53 (s, 1H), 8.35 (s, 1H), 8.29 (s, 1H), 8.03 (s, 1H), 7.97 (dd, J=5.1, 1.4 Hz, 1H), 7.93-7.86 (m, 1H), 7.66 (dd, J=7.2, 1.6 Hz, 1H), 6.50 (t, J=7.2 Hz, 1H), 5.98 (s, 1H), 4.96-4.70 (m, 1H), 4.01 (s, 3H), 3.22-3.16 (m, 1H), 3.07 (d, J=4.4 Hz, 3H), 1.44-1.28 (m, 1H), 1.21-1.11 (m, 1H). LC/MS (ESI, m/z): [(M+H)]⁺=493.2.

### Step 3—3-[(3-{[(1R,2S)-2-fluorocyclopropyl]carbamoyl}-8-(methylamino)imidazo[1,2-b]pyridazin-6-yl)amino]-2-oxo-[1,2′-bipyridine]-4′-carboxylic Acid

To a stirred solution of methyl 3-[(3-{[(1R,2S)-2-fluorocyclopropyl]carbamoyl}-8-(methylamino)imidazo[1,2-b]pyridazin-6-yl)amino]-2-oxo-[1,2′-bipyridine]-4′-carboxylate (2.9 g, 5.9 mmol) in THF (70 mL) and H₂O (70 mL) was added LiOH (1.41 g, 58.9 mmol) at rt. The resulting mixture was stirred for 2 h at rt. On completion, the reaction mixture was concentrated under reduced pressure. The mixture was acidified to pH 2 with conc. HCl. The precipitated solid was collected by filtration. The filter cake was washed with Et₂O (3×10 mL) and dried under vacuum to provide the title compound (2.56 g, 91% yield) as a white solid. ¹H NMR (400 MHz, DMSO-d₆) δ 13.53 (br, s, 1H), 8.82 (d, J=5.1 Hz, 1H), 8.72 (s, 1H), 8.67 (d, J=4.2 Hz, 1H), 8.33 (s, 1H), 8.01 (dd, J=7.5, 1.7 Hz, 1H), 7.97 (s, 1H), 7.93 (dd, J=5.0, 1.4 Hz, 1H), 7.63 (dd, J=7.1, 1.7 Hz, 1H), 7.57 (s, 1H), 6.44 (t, J=7.2 Hz, 1H), 6.41 (s, 1H), 4.98-4.78 (m, 1H), 3.02-2.97 (m, 1H), 2.88 (d, J=3.3 Hz, 3H), 1.29-1.17 (m, 1H), 1.05-0.94 (m, 1H). LC/MS (ESI, m/z): [(M+H)]⁺=479.2.

### Example 1.17. Preparation of 6-({4-[(2-methanesulfonylphenyl)amino]-5-(methylcarbamoyl)pyridin-2-yl}amino)pyridine-3-carboxylic Acid (Intermediate Q)

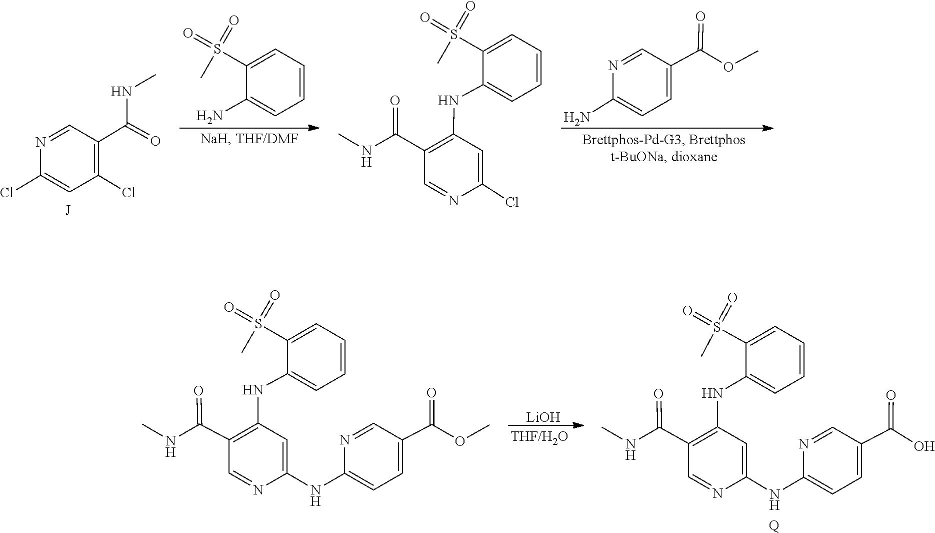

### Step 1—6-chloro-4-[(2-methanesulfonylphenyl)amino]-N-methylpyridine-3-carboxamide

To a stirred mixture of 2-methanesulfonylaniline (751.51 mg, 4.389 mmol) and NaH (589.88 mg, 14.747 mmol, 60% dispersion in mineral oil) in THF (5 mL) at 0° C. under nitrogen atmosphere. Then the mixture was stirred at 0° C. for 30 min. To the above mixture was added 4,6-dichloro-N-methylpyridine-3-carboxamide (600 mg, 2.926 mmol, Intermediate J) at rt. The resulting mixture was stirred for additional 3 h at rt. On completion, the reaction was quenched by the addition of sat. NH₄Cl (aq.) (3 mL) at 0° C. The resulting mixture was diluted with EtOAc (20 mL), then washed with brine (20 mL) and dried over anhydrous Na₂SO₄. After filtration, the filtrate was concentrated under reduced pressure. The residue was purified by reverse phase flash chromatography (Column: WelFlash™ C18-I, 20-40 um, 330 g; Eluent A: Water (plus 10 mmol/L FA); Eluent B: ACN; Gradient: 25%-55% B in 35 min; Flow rate: 70 mL/min; Detector: 254 nm; desired fractions were collected at 36% B) and concentrated under reduced pressure to afford the title compound (750 mg, 75% yield) as a yellow solid. ¹H NMR (400 MHz, DMSO-d₆) δ 10.77 (s, 1H), 8.82-8.78 (m, 1H), 8.58 (s, 1H), 8.00-7.94 (m, 1H), 7.81-7.73 (m, 1H), 7.69 (d, J=8.0 Hz, 1H), 7.51-7.42 (m, 1H), 6.98 (s, 1H), 3.17 (s, 3H), 2.80 (d, J=4.5 Hz, 3H); LC/MS (ESI, m/z): [(M+H)]⁺=340.0.

### Step 2—Methyl 6-({4-[(2-methanesulfonylphenyl)amino]-5-(methylcarbamoyl)pyridin-2-yl}amino)pyridine-3-carboxylate

To a stirred mixture of 6-chloro-4-[(2-methanesulfonylphenyl)amino]-N-methylpyridine-3-carboxamide (447.5 mg, 1.32 mmol/L) and methyl 6-aminopyridine-3-carboxylate (240.8 mg, 1.58 mmol/L) in dioxane (8 mL) were added BrettPhos Pd G3 (120.05 mg, 0.132 mmol), BrettPhos (71.09 mg, 0.132 mmol) and t-BuONa (254.55 mg, 2.648 mmol) in portions at rt under nitrogen atmosphere. The resulting mixture was stirred for 16 h at 100° C. under nitrogen atmosphere. On completion, the reaction mixture was diluted with EtOAc (30 mL), washed with brine (3×20 mL), dried over anhydrous Na₂SO₄. After filtration, the filtrate was concentrated under reduced pressure. The residue was purified by reverse phase flash chromatography (Column: WelFlash™ C18-I, 20-40 um, 120 g; Eluent A: Water (plus 10 mmol/L FA); Eluent B: ACN; Gradient: 20%-55% B in 35 min; Flow rate: 70 mL/min; Detector: 254 nm; desired fractions were collected at 31% B) and concentrated under reduced pressure to afford the title compound (280 mg, 46% yield) as a white solid. ¹H NMR (400 MHz, DMSO-d₆) δ 10.84 (s, 1H), 10.28 (s, 1H), 8.63 (d, J=2.4 Hz, 1H), 8.57 (d, J=12.2 Hz, 2H), 8.15-8.08 (m, 1H), 8.00-7.93 (m, 1H), 7.85-7.74 (m, 1H), 7.78 (s, 2H), 7.68 (d, J=8.9 Hz, 1H), 7.46-7.37 (m, 1H), 3.83 (s, 3H), 3.17 (s, 3H), 2.80 (d, J=4.4 Hz, 3H); LC/MS (ESI, m/z): [(M+H)]⁺=456.1.

### Step 3—6-({4-[(2-methanesulfonylphenyl)amino]-5-(methylcarbamoyl)pyridin-2-yl}amino)pyridine-3-carboxylic Acid

To a stirred mixture of methyl 6-({4-[(2-methanesulfonylphenyl)amino]-5-(methylcarbamoyl)pyridin-2-yl}amino)pyridine-3-carboxylate (150 mg, 0.33 mmol) in THF (5 mL) and H₂O (5 mL) was added LiOH (78.87 mg, 3.290 mmol) in portions at rt under nitrogen atmosphere. The resulting mixture was stirred for 2 h at rt under nitrogen atmosphere. On completion, the reaction mixture was acidified to pH 6 with HCl (aq.). The precipitated solids were collected by filtration. The solid was dried over under reduced pressure to afford the title compound (150 mg, 99% yield) as a white solid. ¹H NMR (400 MHz, DMSO-d₆) δ 13.10 (s, 1H), 11.13 (s, 1H), 10.99 (s, 1H), 8.88 (s, 1H), 8.74 (d, J=2.3 Hz, 1H), 8.60 (d, J=1.4 Hz, 1H), 8.23-8.16 (m, 1H), 8.05-7.98 (m, 1H), 7.90-7.81 (m, 1H), 7.78 (d, J=8.0 Hz, 1H), 7.58-7.50 (m, 1H), 7.41 (d, J=8.7 Hz, 1H), 7.25 (s, 1H), 3.22 (s, 3H), 2.82 (d, J=4.5 Hz, 3H); LC/MS (ESI, m/z): [(M+H)]⁺=442.2.

### Example 1.18. Preparation of 6-((4-((2-methoxyphenyl)amino)-5-(methylcarbamoyl)pyridin-2-yl)amino)nicotinic Acid (Intermediate R)

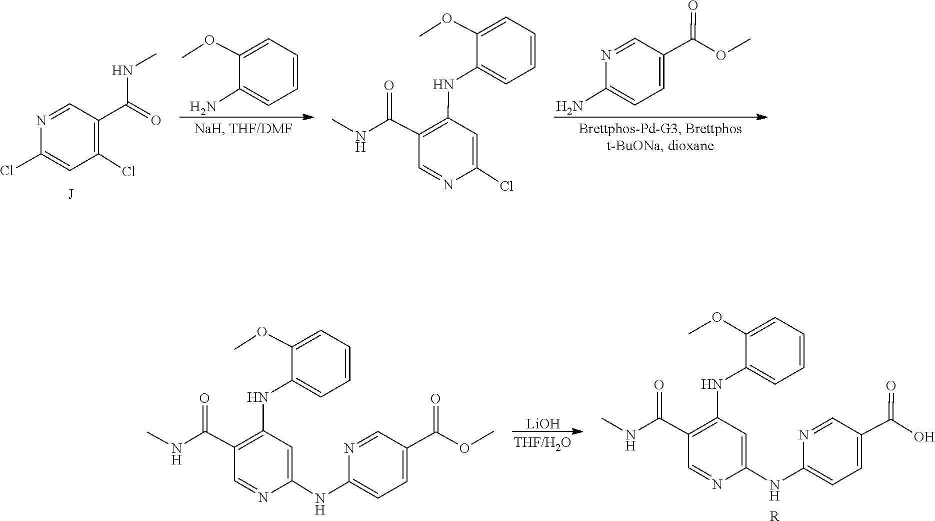

### Step 1—6-Chloro-4-[(2-methoxyphenyl)amino]-N-methylpyridine-3-carboxamide

A mixture of 2-methoxyaniline (540.58 mg, 4.389 mmol) and NaH (351.12 mg, 8.778 mmol, 60% dispersion in mineral oil) in DMF (5 mL) and THF (5 mL) was stirred for 30 min at 0° C. under nitrogen atmosphere. To the above mixture was added 4,6-dichloro-N-methylpyridine-3-carboxamide (600 mg, 3 mmol, Intermediate J) at rt. The resulting mixture was stirred for additional 3 h at rt. On completion, the reaction was quenched with sat. NH4Cl (aq.) (3 mL) at 0° C. The resulting mixture was extracted with EtOAc (3×3 mL). The combined organic layers were washed with brine (3×3 mL), dried over anhydrous Na₂SO₄. After filtration, the filtrate was concentrated under reduced pressure. The residue was purified by reverse phase flash chromatography (Column: WelFlash™ C18-I, 20-40 um, 120 g; Eluent A: Water (plus 10 mmol/L FA); Eluent B: ACN; Gradient: 25%-55% B in 35 min; Flow rate: 70 mL/min; Detector: 254 nm; desired fractions were collected at 34% B) and concentrated under reduced pressure to afford the title compound (480 mg, 56% yield) as a yellow solid. ¹H NMR (400 MHz, DMSO-d₆) δ 10.30 (s, 1H), 8.79-8.71 (m, 1H), 8.48 (s, 1H), 7.42-7.35 (m, 1H), 7.26-7.18 (m, 1H), 7.18-7.12 (m, 1H), 7.06-6.97 (m, 1H), 6.77 (s, 1H), 3.83 (s, 3H), 2.80 (d, J=4.4 Hz, 3H). LC/MS (ESI, m/z): [(M+H)]⁺=292.1.

### Step 2—Methyl 6-((4-((2-methoxyphenyl)amino)-5-(methylcarbamoyl)pyridin-2-yl)amino)nicotinate

To a stirred mixture of 6-chloro-4-[(2-methoxyphenyl)amino]-N-methylpyridine-3-carboxamide (480 mg, 1.65 mmol) and methyl 6-aminopyridine-3-carboxylate (375.51 mg, 2.468 mmol, CAS #36052-24-1) in dioxane (8 mL) were added BrettPhos Pd G3 (149.15 mg, 0.165 mmol) and t-BuONa (316.24 mg, 3.290 mmol) at rt under nitrogen atmosphere. The resulting mixture was stirred for 16 h at 10° C. under nitrogen atmosphere. On completion, the reaction mixture was diluted with EtOAc (20 mL), then washed with brine (2×10 mL), and dried over anhydrous Na₂SO₄. After filtration, the filtrate was concentrated under reduced pressure. The residue was purified by reverse phase flash chromatography (Column: WelFlash™ C18-I, 20-40 um, 330 g; Eluent A: Water (plus 10 mmol/L FA); Eluent B: ACN; Gradient: 25%-55% B in 35 min; Flow rate: 80 mL/min; Detector: 254 nm; desired fractions were collected at 32% B) and concentrated under reduced pressure to afford the title compound (550 mg, 82% yield) as a white solid. ¹H NMR (400 MHz, DMSO-d₆) δ 11.00 (m, 1H), 10.59 (s, 1H), 8.84-8.78 (m, 2H), 8.47 (s, 1H), 8.25-8.21 (m, 1H), 7.55-7.48 (m, 1H), 7.48-7.18 (m, 3H), 7.16-7.04 (m, 1H), 6.97-6.93 (m, 1H), 3.87 (s, 3H), 3.86 (s, 3H), 2.82 (d, J=4.4 Hz, 3H). LC/MS (ESI, m/z): [(M+H)]⁺=408.2.

### Step 3—6-((4-((2-methoxyphenyl)amino)-5-(methylcarbamoyl)pyridin-2-yl)amino)nicotinic Acid

To a stirred mixture of methyl 6-({4-[(2-methoxyphenyl)amino]-5-(methylcarbamoyl)pyridin-2-yl}amino)pyridine-3-carboxylate (250 mg, 0.614 mmol) in THF (5 mL) and H₂O (5 mL) was added LiOH (146.96 mg, 6.140 mmol) at rt under nitrogen atmosphere. The resulting mixture was stirred for 2 h at rt under nitrogen atmosphere. On completion, the reaction mixture was concentrated under reduced pressure. The mixture was acidified to pH 6 with HCl (aq.). The precipitated solids were collected by filtration and washed with H₂O (2×2 mL). The solid was dried over under reduced pressure to afford the title compound (200 mg, 83% yield) as a white solid. ¹H NMR (400 MHz, DMSO-d₆) δ 13.22 (s, 1H), 11.21 (s, 1H), 10.62 (s, 1H), 8.89 (s, 1H), 8.80 (d, J=2.3 Hz, 1H), 8.47 (d, J=1.4 Hz, 1H), 8.28-8.20 (m, 1H), 7.50-7.43 (m, 1H), 7.35-7.27 (m, 1H), 7.27-7.19 (m, 2H), 7.13-7.05 (m, 1H), 6.92 (s, 1H), 3.86 (s, 3H), 2.82 (d, J=4.4 Hz, 3H). LC/MS (ESI, m/z): [(M+H)]⁺=394.2.

### Example 1.19. Preparation of 4-{[3-(5-fluoropyrimidin-2-yl)-2-methoxyphenyl]amino}-6-[(5-formylpyridin-2-yl)amino]-N-methylpyridine-3-carboxamide (Intermediate S)

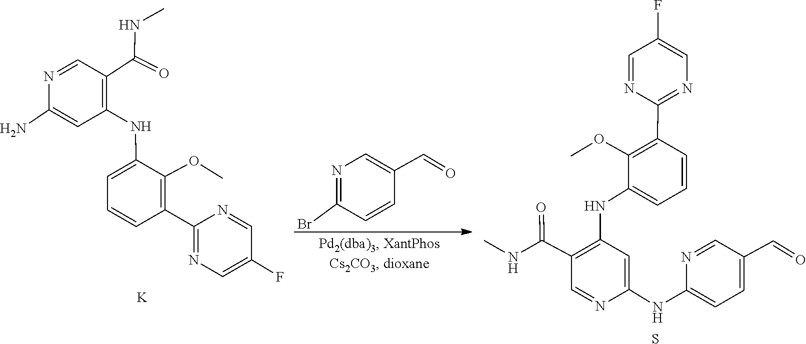

To a stirred mixture of 6-chloro-4-((3-(5-fluoropyrimidin-2-yl)-2-methoxyphenyl)amino)-N-methylnicotinamide (6 g, 15 mmol, Intermediate K) and 6-bromonicotinaldehyde (2.83 g, 23.2 mmol, CAS #149806-06-4) in 1,4-dioxane (100 mL) were added BrettPhos (0.83 g, 1.5 mmol), BrettPhos Pd G3 (1.40 g, 1.55 mmol) and t-BuONa (2.97 g, 30.9 mmol) in turns at rt under nitrogen atmosphere. The resulting mixture was stirred for 1 h at 120° C. under nitrogen atmosphere. On completion, the mixture was cooled to rt and the mixture was filtered. The filter cake was washed with DCM (3×50 mL). The filtrate was concentrated under reduced pressure. The residue was dissolved in water (100 mL) and then was extracted with EtOAc (3×200 mL). The combined organic layers were washed with brine (1×500 mL), and dried over anhydrous Na₂SO₄. After filtration, the filtrate was concentrated under reduced pressure. The residue was purified by reverse phase flash chromatography (Column: WelFlash™ C18-I, 20-40 um, 330 g; Eluent A: Water (plus 10 mmol/L NH₄HCO₃); Eluent B: ACN; Gradient: 25%-55% B in 25 min; Flow rate: 80 mL/min; Detector: 220/254 nm; desired fractions were collected at 55% B) and concentrated under reduced pressure to afford the title compound (5.1 g, 70% yield) as a white solid. ¹H NMR (400 MHz, DMSO-d₆) δ 10.73 (s, 1H), 10.44 (s, 1H), 9.88 (s, 1H), 9.04 (s, 2H), 8.77-8.67 (m, 1H), 8.63-8.58 (m, 1H), 8.56 (s, 1H), 8.10-8.01 (m, 2H), 7.79-7.67 (m, 2H), 7.48-7.42 (m, 1H), 7.39-7.31 (m, 1H), 3.71 (s, 3H), 2.81 (d, J=4.4 Hz, 3H); LC/MS (ESI, m/z): [(M+H)]⁺=474.1.

### Example 1.20. Preparation of 3-[3-methyl-2-oxo-5-(piperazin-1-yl)-1,3-benzodiazol-1-yl]piperidine-2,6-dione (Intermediate T)

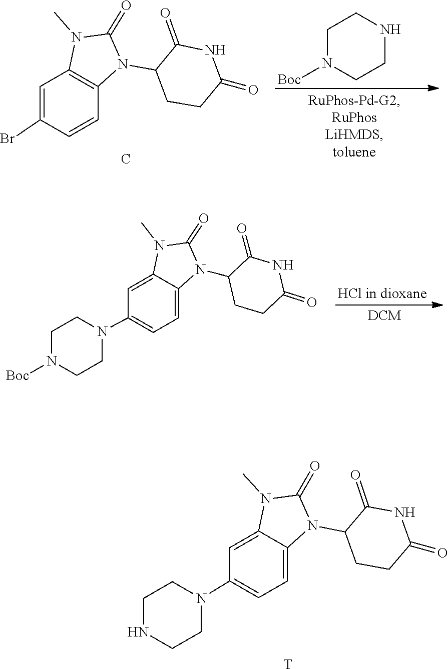

### Step 1—Tert-butyl 4-[1-(2,6-dioxopiperidin-3-yl)-3-methyl-2-oxo-1,3-benzodiazol-5-yl]piperazine-1-carboxylate

To a stirred mixture of 3-(5-bromo-3-methyl-2-oxo-1,3-benzodiazol-1-yl)piperidine-2,6-dione (2 g, 5.9 mmol, Intermediate C) and tert-butyl piperazine-1-carboxylate (1.65 g, 8.87 mmol) in toluene (35.5 mL) were added RuPhos (0.55 g, 1.18 mmol) and RuPhos-PdCl-2nd G (0.92 g, 1.18 mmol), followed by dropwise addition of LiHMDS (35.5 mL, 35.5 mmol) at rt under nitrogen atmosphere. The resulting mixture was stirred for 1 h at 80° C. under nitrogen atmosphere. On completion, the reaction was acidified to pH 6 with FA, followed by concentration under reduced pressure. The residue was purified by reverse phase flash chromatography (Column: WelFlash™ C18-I, 20-40 um, 330 g; Eluent A: Water (plus 10 mmol/L FA); Eluent B: ACN; Gradient: 25%-55% B in 25 min; Flow rate: 80 mL/min; Detector: 220/254 nm; desired fractions were collected at 38% B) and concentrated under reduced pressure to afford the title compound (900 mg, 34% yield) as a brown solid. LC/MS (ESI, m/z): [(M+1)]⁺=444.3.

### Step 2—3-[3-Methyl-2-oxo-5-(piperazin-1-yl)-1,3-benzodiazol-1-yl]piperidine-2,6-dione

To a solution of tert-butyl 4-[1-(2,6-dioxopiperidin-3-yl)-3-methyl-2-oxo-1,3-benzodiazol-5-yl]piperazine-1-carboxylate (1 g, 2 mmol) in DCM (20 mL) was added 4 M HCl (gas) in 1,4-dioxane (10 mL) dropwise at rt. The resulting mixture was stirred for 1 h at rt. The resulting mixture was concentrated under reduced pressure. The residue product was purified by reverse phase flash (Column: Spherical C18 Column, 20-40 um, 120 g; Mobile Phase A: Water (5 mmol/L NH₄HCO₃), Mobile Phase B: ACN; Flow rate: 60 mL/min; Gradient: 5% B to 35% B in 25 min, 254 nm; the fractions containing the desired product were collected at 30% B) to afford the title compound (700 mg, 90% yield) as a white solid. LC/MS (ESI, m/z): [(M+1)]⁺=344.3.

### Example 1.21. Preparation of 3-(4-Bromo-3-methyl-2-oxo-2,3-dihydro-1H-benzo[d]imidazol-1-yl)piperidine-2,6-dione (Intermediate U)

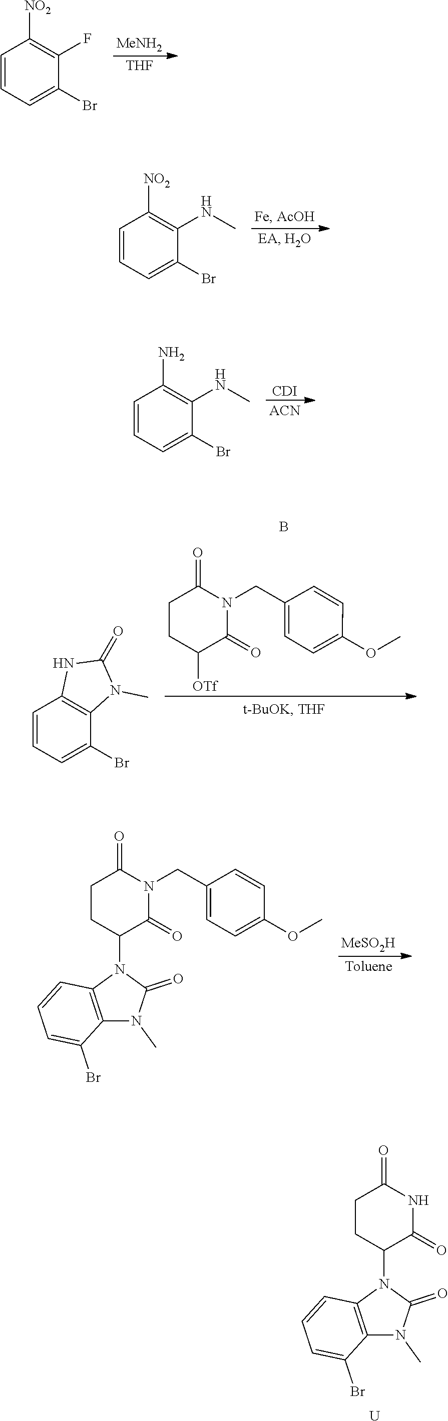

### Step 1—2-Bromo-N-methyl-6-nitro-aniline

To a solution of 1-bromo-2-fluoro-3-nitro-benzene (40.0 g, 181 mmol, CAS #58534-94-4) in THF (40 mL) was added MeNH₂ (2 M, 400 mL). The reaction mixture was stirred at 60° C. for 12 hours. On completion, the reaction mixture was poured into sat.NaHCO₃ (30 mL) and extracted with EA (3×200 mL). The combined organic layers were washed with brine (2×200 mL), dried with anhydrous Na₂SO₄, filtered and concentrated in vacuo to give the title compound (40.0 g, 95% yield) as red oil. LC-MS (ESI⁺) m/z 230.9 (M+H)⁺.

### Step 2—3-Bromo-N2-methyl-benzene-1,2-diamine

To a mixture of 2-bromo-N-methyl-6-nitro-aniline (23.0 g, 99.5 mmol) in EA (300 mL) and H₂O (10 mL) was added AcOH (100 mL). The mixture was warmed to 50° C. Then Fe (22.2 g, 398 mmol) was added to the reaction mixture and the mixture was heated to 80° C. about 4 hours. On completion, the reaction mixture was filtered and concentrated in vacuo. The residue was diluted with water (100 mL) and extracted with EA (3×200 mL). The combined organic layers was dried over Na₂SO₄, filtered and concentrated in vacuo to give the title compound (20.0 g, 99% yield) as red oil. ¹H NMR (400 MHz, DMSO-d₆) δ 6.73-6.70 (m, 1H), 6.68-6.60 (m, 2H), 5.02 (s, 2H), 3.67 (s, 1H), 2.58 (s, 3H).

### Step 3—4-Bromo-3-methyl-1H-benzimidazol-2-one

To a mixture of 3-bromo-N2-methyl-benzene-1,2-diamine (20.0 g, 99.4 mmol) in ACN (300 mL) was added CDI (32.2 g, 198 mmol). The reaction mixture was stirred at 85° C. for 12 hours under N₂ atmosphere. On completion, the reaction mixture was concentrated in vacuo. The reaction mixture was diluted with water (200 mL), where a solid precipitate was formed, which was filtered off. The solid was washed with water (1 L) and dried in vacuo to give the title compound (20.0 g, 88% yield) as white solid. ¹H NMR (400 MHz, DMSO-d₆) δ 11.17 (s, 1H), 7.14 (dd, J=1.2, 8.0 Hz, 1H), 7.00-6.95 (m, 1H), 6.93-6.87 (m, 1H), 3.55 (s, 3H).

### Step 4—3-(4-Bromo-3-methyl-2-oxo-benzimidazol-1-yl)-1-[(4-methoxyphenyl)methyl]piperidine-2,6-dione

To a solution of 4-bromo-3-methyl-1H-benzimidazol-2-one (12.0 g, 52.8 mmol) in THF (300 mL) was added t-BuOK (7.12 g, 63.4 mmol). The reaction mixture was stirred at 0° C. for 0.5 hr. Subsequently, [1-[(4-methoxyphenyl)methyl]-2,6-dioxo-3-piperidyl]trifluoromethanesulfonate (20.1 g, 52.8 mmol, Intermediate B) in a solution of THF (100 mL) was added dropwise. The resulting reaction mixture was stirred at 20° C. for 0.5 hr under N₂. On completion, the reaction mixture was quenched with saturated NH₄Cl (100 mL), and extracted with ethyl acetate (200 mL). The combined organic layers were washed with brine (2×100 mL), dried over anhydrous sodium sulfate, filtered, the filtrate was concentrated in vacuo. The crude product was purified by reversed-phase HPLC (0.1% FA condition) to give the title compound (13.3 g, 55% yield) as a yellow solid. ¹H NMR (400 MHz, CDCl₃) δ 7.38 (d, J=8.8 Hz, 2H), 7.22 (d, J=8.0 Hz, 1H), 6.84 (d, J=8.8 Hz, 2H), 6.80 (t, J=8.0 Hz, 1H), 6.48-6.40 (d, J=8.0 Hz, 1H), 5.22 (dd, J=5.2, 12.8 Hz, 1H), 5.04-4.93 (m, 2H), 3.81 (s, 3H), 3.80 (s, 3H), 3.12-2.98 (m, 1H), 2.93-2.77 (m, 1H), 2.62 (dq, J=4.4, 13.2 Hz, 1H), 2.20-2.17 (m, 1H).

### Step 5—3-(4-bromo-3-methyl-2-oxo-2,3-dihydro-1H-benzo[d]imidazol-1-yl)piperidine-2,6-dione

A mixture of 3-(4-bromo-3-methyl-2-oxo-benzimidazol-1-yl)-1-[(4-methoxyphenyl)methyl]piperidine-2,6-dione (13.3 g, 29.0 mmol) in a mixed solvent of Tol. (80 mL) and methane sulfonic acid (40 mL) was degassed and purged with N₂ for 3 times, and then the mixture was stirred at 120° C. for 2 hrs under N₂ atmosphere. On completion, the reaction mixture was concentrated in vacuo to remove toluene. The residue was added 200 mL of ice water, and then white solid precipitate formed. The mixture was filtered and the filtered cake was collected and dried over in vacuo to give the title compound (7.30 g, 74% yield) as white solid. ¹H NMR (400 MHz, DMSO-d₆) δ 11.13 (s, 1H), 7.25 (d, J=8.0 Hz, 1H), 7.17 (d, J=8.0 Hz, 1H), 7.05-6.93 (m, 1H), 5.41 (dd, J=5.2, 12.8 Hz, 1H), 3.64 (s, 3H), 2.96-2.83 (m, 1H), 2.78-2.59 (m, 2H), 2.08-2.00 (m, 1H).

### Example 1.22. Preparation of 3-(3-methyl-4-(4-(methylamino)piperidin-1-yl)-2-oxo-2,3-dihydro-1H-benzo[d]imidazol-1-yl)piperidine-2,6-dione (Intermediate V)

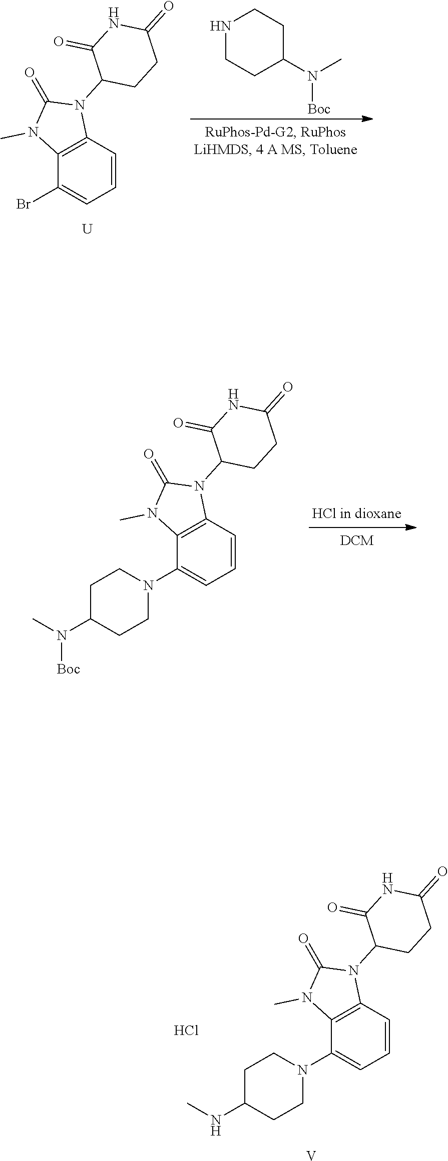

### Step 1—Tert-butyl (1-(1-(2,6-dioxopiperidin-3-yl)-3-methyl-2-oxo-2,3-dihydro-1H-benzo[d]imidazol-4-yl)piperidin-4-yl)(methyl)carbamate

To a stirred solution of 3-(4-bromo-3-methyl-2-oxo-1,3-benzodiazol-1-yl)piperidine-2,6-dione (2 g, 6 mmol, Intermediate U) and tert-butyl N-methyl-N-(piperidin-4-yl)carbamate (1.90 g, 8.87 mmol) in toluene (30 mL) were added RuPhos-PdCl-2nd G (0.92 g, 1.2 mmol) and RuPhos (0.55 g, 1.2 mmol), followed by added LiHMDS (35.49 mL, 35.48 mmol) dropwise at rt under N₂ atmosphere. The resulting mixture was stirred at 80° C. under nitrogen atmosphere. On completion, the mixture was acidified to pH 3 with HCl (aq.2 mol/L). The resulting mixture was filtered and the filter cake was washed with MeCN (3×30 mL). The filtrate was concentrated under reduced pressure. The residue was purified by reverse flash chromatography (column, C18 silica gel; mobile phase A: Water (0.1% FA), mobile phase B: MeCN, 30% to 70% gradient in 30 min; detector, UV 254 nm) to afford the title compound (1.6 g, 57% yield) as a brown solid. ¹H NMR (400 MHz, DMSO-d₆) δ 11.08 (s, 1H), 7.04-6.82 (m, 3H), 5.35 (dd, J=12.6, 5.4 Hz, 1H), 3.65 (s, 3H), 3.18-3.13 (m, 2H), 2.97-2.58 (m, 9H), 2.10-1.78 (m, 3H), 1.71-1.52 (m, 2H), 1.43 (s, 9H); LC/MS (ESI, m/z): [(M+H)]*=472.2.

### Step 2—3-(3-methyl-4-(4-(methylamino)piperidin-1-yl)-2-oxo-2,3-dihydro-1H-benzo[d]imidazol-1-yl)piperidine-2,6-dione Hydrochloride

To a stirred solution of tert-butyl (1-(1-(2,6-dioxopiperidin-3-yl)-3-methyl-2-oxo-2,3-dihydro-1H-benzo[d]imidazol-4-yl)piperidin-4-yl)(methyl)carbamate (800 mg, 2 mmol) in DCM (10 mL) was added 4 M HCl (gas) in 1,4-dioxane (5 mL) at rt. The reaction mixture was stirred for 1 h at rt under nitrogen atmosphere. On completion, the reaction mixture was concentrated under reduced pressure. The residue was triturated with Et₂O (15 mL) to afford the title compound (660 mg, 95% yield) as a yellow solid. ¹H NMR (400 MHz, DMSO-d₆) δ 11.03 (broad, 1H), 8.36 (s, 1H), 7.04-6.85 (m, 3H), 5.35 (dd, J=12.6, 5.3 Hz, 1H), 3.63 (s, 3H), 3.18-3.13 (m, 2H), 2.97-2.56 (m, 6H), 2.45 (s, 3H), 2.10-1.91 (m, 3H), 1.65-1.59 (m, 2H); LC/MS (ESI, m/z): [(M+H)]⁺=372.2.

### Example 1.23. Preparation of 3-(3-Methyl-5-(4-(methylamino)piperidin-1-yl)-2-oxo-2,3-dihydro-1H-benzo[d]imidazol-1-yl)piperidine-2,6-dione (Intermediate W)

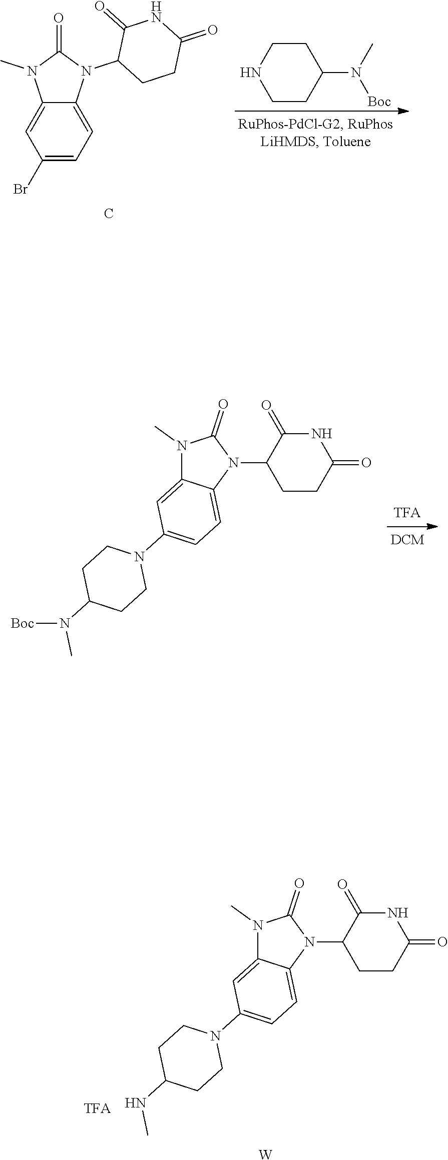

### Step 1—Tert-butyl N-{1-[1-(2,6-dioxopiperidin-3-yl)-3-methyl-2-oxo-1,3-benzodiazol-5-yl]piperidin-4-yl}-N-methylcarbamate

To a stirred mixture of 3-(5-bromo-3-methyl-2-oxo-1,3-benzodiazol-1-yl)piperidine-2,6-dione (2 g, 6 mmol, Intermediate C) and tert-butyl N-methyl-N-(piperidin-4-yl)carbamate (1.90 g, 8.87 mmol) in toluene (30 mL) were added RuPhos (0.55 g, 1.2 mmol) and RuPhos-PdCl-2nd G (0.92 g, 1.2 mmol) at rt under nitrogen atmosphere. To the above mixture was added LiHMDS (35 mL, 35 mmol) dropwise at rt. The resulting mixture was stirred for additional 4 h at 80° C. On completion, the mixture was cooled to rt and acidified to pH 6 with FA and filtered. The filter cake was washed with DCM (2×50 mL). The filtrate was concentrated under reduced pressure. The residue was purified by reverse flash chromatography (column, C18 silica gel; mobile phase A: water (0.1% FA), mobile phase B: MeCN in water, 30% to 60% gradient in 30 min; detector, UV 220/254 nm; desired fractions were collected at 52% B) and concentrated under reduced pressure to afford the title compound (1.9 g, 68% yield) as a purple solid. ¹H NMR (400 MHz, Chloroform-d) δ 8.56 (s, 1H), 6.76-6.67 (m, 3H), 5.26-5.17 (m, 1H), 4.21-4.16 (m, 1H), 3.67-3.58 (m, 2H), 3.46-3.37 (m, 3H), 3.00-2.84 (m, 2H), 2.84-2.80 (m, 3H), 2.79-2.64 (m, 3H), 2.28-2.17 (m, 1H), 1.95-1.88 (m, 2H), 1.82-1.74 (m, 2H), 1.50 (s, 9H); LC/MS (ESI, m/z): [(M+H)]⁺=472.3.

### Step 2—3-(3-Methyl-5-(4-(methylamino) piperidin-1-yl)-2-oxo-2,3-dihydro-1H-benzo[d]imidazol-1-yl)piperidine-2,6-dione Trifluoroacetate

To a stirred solution of tert-butyl N-{1-[1-(2,6-dioxopiperidin-3-yl)-3-methyl-2-oxo-1,3-benzodiazol-5-yl]piperidin-4-yl}-N-methylcarbamate (900 mg, 1.9 mmol) in DCM (18 mL) was added TFA (6 mL) at rt and the mixture was stirred for 1 h at rt. On completion, the reaction mixture was concentrated under reduced pressure. The residue was triturated with diethyl ether (50 mL) to give the title compound (600 mg) as a yellow solid. LC/MS (ESI, m/z): [(M+H)]⁺=372.2.

### Example 1.24. Preparation of 6-{[5-(ethylcarbamoyl)-4-{[3-(5-fluoropyrimidin-2-yl)-2-methoxyphenyl]amino}pyridin-2-yl]amino}pyridine-3-carboxylic Acid (Intermediate X)

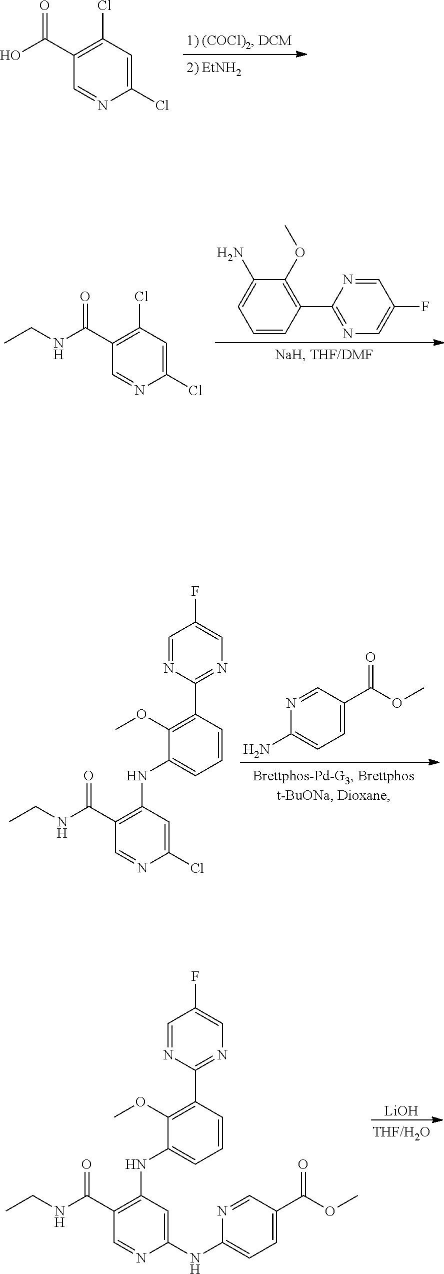

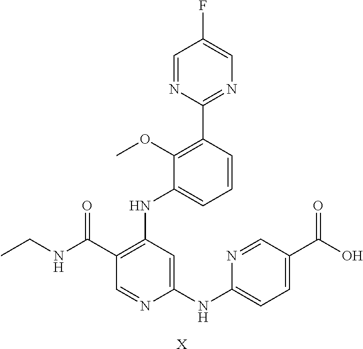

### Step 1—4,6-Dichloro-N-ethylnicotinamide

To a stirred mixture of 4,6-dichloropyridine-3-carboxylic acid (5 g, 26 mmol) and (CO)₂Cl₂ (3.97 g, 31.3 mmol) in DCM (100 mL) was added DMF (0.20 mL, 3 mmol) at rt under nitrogen atmosphere. The resulting mixture was stirred for 30 min at rt under nitrogen atmosphere. On completion, the reaction mixture was concentrated under vacuum. The residue was re-dissolved in DCM (50 mL), then a solution of ethylamine (2.0 M in THF, 15.63 mL, 31.25 mmol) was added dropwise to the mixture over 10 min at rt. The resulting mixture was stirred for 2 h at rt under nitrogen atmosphere. On completion, the reaction mixture was concentrated then the residue was purified by silica gel column chromatography, eluted with PE/EA (1:1), to afford the title compound (5.5 g, 96% yield) as a white solid. ¹H NMR (400 MHz, DMSO-d₆) δ 8.66 (t, J=5.5 Hz, 1H), 8.47 (t, J=1.8 Hz, 1H), 7.90 (d, J=1.7 Hz, 1H), 3.30-3.22 (m, 2H), 1.12 (t, J=7.2 Hz, 3H). LC/MS (ESI, m/z): [(M+H)]⁺=219.0.

### Step 2—6-Chloro-N-ethyl-4-{[3-(5-fluoropyrimidin-2-yl)-2-methoxyphenyl]amino}pyridine-3-carboxamide

To a stirred mixture of 4,6-dichloro-N-ethylpyridine-3-carboxamide (2.398 g, 10.95 mmol) and 3-(5-fluoropyrimidin-2-yl)-2-methoxyaniline (2 g, 9 mmol, synthesized via Steps 1-2 of Intermediate K) in NMP (3 mL) was added TsOH (2.356 g, 13.68 mmol) at rt under nitrogen atmosphere. The resulting mixture was stirred for 2 h at 100° C. under nitrogen atmosphere. On completion, the mixture was cooled to rt and concentrated under reduced pressure. The crude product was purified by reverse phase flash chromatography (Column: WelFlash™ C18-I, 20-40 um, 330 g; Eluent A: Water (10 mmol/L NH₄HCO₃); Eluent B: ACN; Gradient: 25%-55% B in 35 min; Flow rate: 80 mL/min; Detector: 254 nm; desired fractions were collected at 50% B) and concentrated under reduced pressure to afford the title compound (1.3 g, 36% yield) as a yellow solid. ¹H NMR (400 MHz, Chloroform-d) δ 10.25 (s, 1H), 8.76 (d, J=1.3 Hz, 2H), 8.38 (s, 1H), 7.66 (dd, J=7.8, 1.6 Hz, 1H), 7.48 (dd, J=7.9, 1.9 Hz, 1H), 7.32-7.24 (m, 1H), 7.00 (d, J=1.4 Hz, 1H), 6.49 (s, 1H), 3.78 (s, 3H), 3.57-3.48 (m, 2H), 1.32-1.15 (m, 3H). LC/MS (ESI, m/z): [(M+H)]⁺=402.1.

### Step 3—Methyl 6-{[5-(ethylcarbamoyl)-4-{[3-(5-fluoropyrimidin-2-yl)-2-methoxyphenyl]amino}pyridin-2-yl]amino}pyridine-3-carboxylate

To a stirred mixture of 6-chloro-N-ethyl-4-{[3-(5-fluoropyrimidin-2-yl)-2-methoxyphenyl]amino}pyridine-3-carboxamide (400 mg, 1 mmol) and methyl 6-aminopyridine-3-carboxylate (227.19 mg, 1.492 mmol) in dioxane (10 mL) were added BrettPhos Pd G3 (90.24 mg, 0.100 mmol) and BrettPhos (53.43 mg, 0.100 mmol) and t-BuONa (191.33 mg, 1.990 mmol) at rt under nitrogen atmosphere. The resulting mixture was stirred for 2 h at 120° C. under nitrogen atmosphere. On completion, the reaction mixture was cooled to rt and concentrated under vacuum. The residue was purified by silica gel column chromatography, eluted with CH₂Cl₂/MeOH (10:1), to afford the title compound (450 mg, 87% yield) as a white solid. ¹H NMR (400 MHz, Chloroform-d) δ 10.34 (s, 1H), 8.76 (d, J=1.3 Hz, 2H), 8.37 (s, 1H), 8.20-8.12 (m, 1H), 7.64-7.54 (m, 3H), 7.46 (d, J=8.8 Hz, 1H), 7.34-7.23 (m, 3H), 3.92 (s, 3H), 3.78 (s, 3H), 3.54-3.48 (m, 2H), 1.32-1.28 (m, 3H). LC/MS (ESI, m/z): [(M+H)]+=518.2.

### Step 4—6-{[5-(ethylcarbamoyl)-4-{[3-(5-fluoropyrimidin-2-yl)-2-methoxyphenyl]amino}pyridin-2-yl]amino}pyridine-3-carboxylic Acid

To a stirred mixture of methyl 6-{[5-(ethylcarbamoyl)-4-{[3-(5-fluoropyrimidin-2-yl)-2-methoxyphenyl]amino}pyridin-2-yl]amino}pyridine-3-carboxylate (370 mg, 0.715 mmol) and LiOH (171.23 mg, 7.150 mmol) in THF (5 mL) and H₂O (5 mL) at rt under nitrogen atmosphere. The resulting mixture was stirred for 1 h at rt under nitrogen atmosphere. On completion, the mixture was acidified to pH 7 with HCl (aq.). The precipitated solids were collected by filtration and washed with CH₂Cl₂ (3×6 mL). The solid was dried under vacuum to give the title compound (300 mg, 83% yield) as a white solid. ¹H NMR (400 MHz, DMSO-d₆) δ 12.87 (s, 1H), 10.68 (s, 1H), 10.27 (s, 1H), 9.04 (s, 2H), 8.70 (s, 1H), 8.62 (s, 1H), 8.54 (s, 1H), 8.12-8.08 (m, 1H), 7.93 (s, 1H), 7.73-7.68 (m, 1H), 7.66 (d, J=8.8 Hz, 1H), 7.48-7.43 (m, 1H), 7.36-7.31 (m, 1H), 3.69 (s, 3H), 3.30-3.27 (m, 2H), 1.18-1.13 (m, 3H). LC/MS (ESI, m/z): [(M+H)]⁺=504.2.

### Example 1.25. Preparation of 3-(3-Methyl-2-oxo-5-(4,4,5,5-tetramethyl-1,3,2-dioxaborolan-2-yl)-2,3-dihydro-1H-benzo[d]imidazol-1-yl)piperidine-2,6-dione (Intermediate Y)

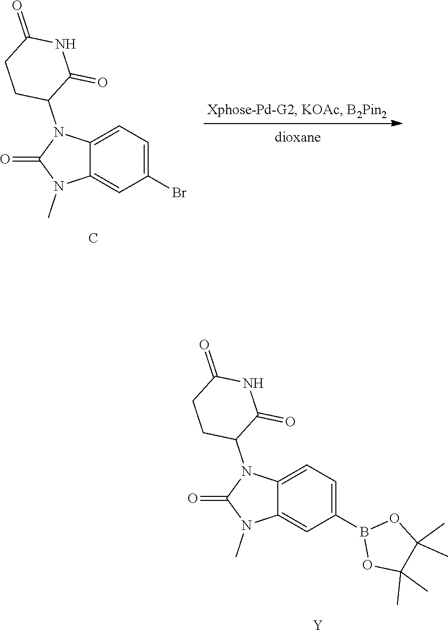

A mixture of 3-(5-bromo-3-methyl-2-oxo-2,3-dihydro-1H-benzo[d]imidazol-1-yl)piperidine-2,6-dione (25 g, 73.9 mmol, Intermediate C), 4,4,4′,4′,5,5,5′,5′-octamethyl-2,2′-bi(1,3,2-dioxaborolane) (20.7 g, 81.3 mmol), Xphos-Pd-G2 (5.82 g, 7.39 mmol) and potassium acetate (21.8 g, 222 mmol) in dioxane (500 mL) was degassed and purged with nitrogen three times, and then the mixture was stirred at 90° C. for 16 hours under nitrogen atmosphere. On completion, the reaction mixture was filtered, then diluted with water (500 mL) and extracted with water (500 mL×2). The combined organic layer was dried over anhydrous sodium sulfate, filtered and concentrated under reduced pressure to give a residue. The crude product was triturated with ethyl acetate (100 mL) at 25° C. for 30 minutes to give the title compound (24 g, 84% yield) as white solid. ¹H NMR (400 MHz, DMSO-d₆) δ=11.12 (s, 1H), 7.44-7.38 (m, 2H), 7.16 (d, J=8.0 Hz, 1H), 5.41 (dd, J=5.4, 12.8 Hz, 1H), 3.38 (s, 3H), 2.96-2.85 (m, 1H), 2.77-2.59 (m, 2H), 2.08-1.99 (m, 1H), 1.31 (s, 12H); LC-MS (ESI+) m/z 386.1 (M+H)⁺.

### Example 1.26. Preparation of tert-butyl 4-((1-(4-bromophenyl)piperidin-4-yl)methyl)piperazine-1-carboxylate (Intermediate Z)

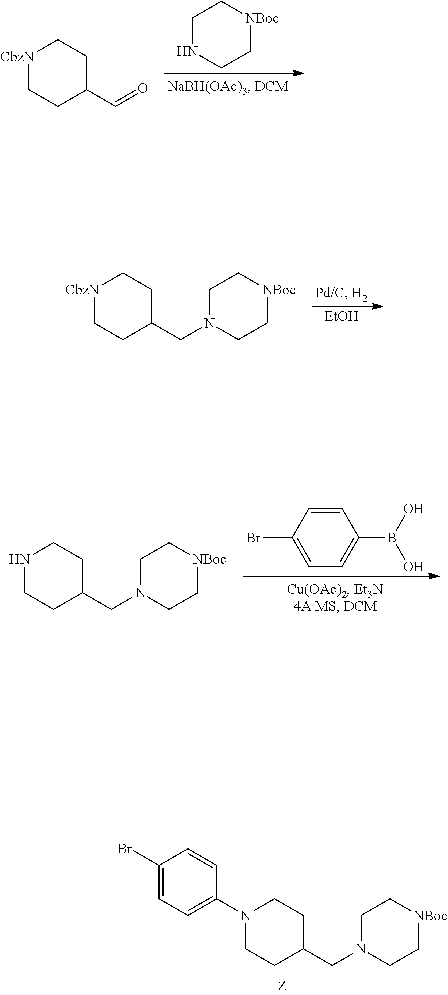

### Step 1—Tert-butyl 4-((1-((benzyloxy)carbonyl)piperidin-4-yl)methyl)piperazine-1-carboxylate

To a solution of benzyl 4-formylpiperidine-1-carboxylate (5.0 g, 20.2 mmol) and tert-butyl piperazine-1-carboxylate (3.80 g, 20.4 mmol) in dichloromethane (100 mL) at 25° C. was added sodium triacetoxy borohydride (5.15 g, 24.3 mmol) in portions, then the reaction mixture was stirred at 25° C. for 12 hours. On completion, the reaction mixture was quenched with water (20 mL) at 20° C., and then diluted with water (80 mL) and extracted with dichloromethane (150 mL×2). The combined organic layers were washed with brine (200 mL×2), dried over anhydrous sodium sulfate, filtered and concentrated under reduced pressure to give a residue. The residue was purified by column chromatography (SiO₂, Petroleum ether/Ethyl acetate=20/1 to 0/1) to give the title compound (7.5 g, 88% yield) as colorless oil. ¹H NMR (400 MHz, CHLOROFORM-d) δ=7.32-7.21 (m, 5H), 5.05 (s, 2H), 4.11 (s, 2H), 3.36 (s, 4H), 2.70 (s, 2H), 2.30 (s, 4H), 2.19-2.07 (m, 2H), 1.73-1.56 (m, 3H), 1.39 (s, 9H), 1.10-0.97 (m, 2H); LC-MS (ESI+) m/z 418.1 (M+H)⁺.

### Step 2—Tert-butyl 4-(piperidin-4-ylmethyl)piperazine-1-carboxylate

To a solution of palladium on carbon (1 g, 10 wt %) in ethanol (30 mL) was added tert-butyl 4-((1-((benzyloxy)carbonyl)piperidin-4-yl)methyl)piperazine-1-carboxylate (3 g, 7.18 mmol) under nitrogen. The suspension was degassed under vacuum and purged with hydrogen several times. The mixture was stirred under hydrogen (15 psi) at 25° C. for 12 hours. On completion, the reaction mixture was filtered and concentrated under reduced pressure to give the title compound (2 g) as colorless oil. ¹H NMR (400 MHz, CDCl₃-d) δ=3.37-3.29 (m, 4H), 3.12 (d, J=12.4 Hz, 2H), 2.62-2.55 (m, 2H), 2.26 (t, J=4.8 Hz, 4H), 2.11 (d, J=7.2 Hz, 2H), 1.74 (d, J=13.2 Hz, 2H), 1.76-1.56 (m, 1H), 1.39 (s, 9H), 1.23-1.11 (m, 2H).

### Step 3—Tert-butyl 4-((1-(4-bromophenyl)piperidin-4-yl)methyl)piperazine-1-carboxylate

A mixture of tert-butyl 4-(piperidin-4-ylmethyl)piperazine-1-carboxylate (1.8 g, 6.35 mmol), (4-bromophenyl)boronic acid (1.53 g, 7.62 mmol), copper acetate (1.73 g, 9.53 mmol), triethylamine (2.57 g, 25.4 mmol) and 4 Å molecular sieve (2 g) in dichloromethane (40 mL) was degassed and purged with oxygen for three times, Then the mixture was stirred at 25° C. for 12 hours under oxygen atmosphere. On completion, the reaction mixture was filtered and concentrated under reduced pressure to give a residue. The residue was purified by column chromatography (SiO₂, Petroleum ether/Ethyl acetate=50/1 to 0/1) to give the title compound (2 g, 71% yield over 2 steps) as off-white solid. LC-MS (ESI+) m/z 440.1 (M+1)⁺.

### Example 1.27. Preparation of 3-(3-methyl-2-oxo-5-(4-(4-(piperazin-1-ylmethyl)piperidin-1-yl)phenyl)-2,3-dihydro-1H-benzo[d]imidazol-1-yl)piperidine-2,6-dione (Intermediate AA)

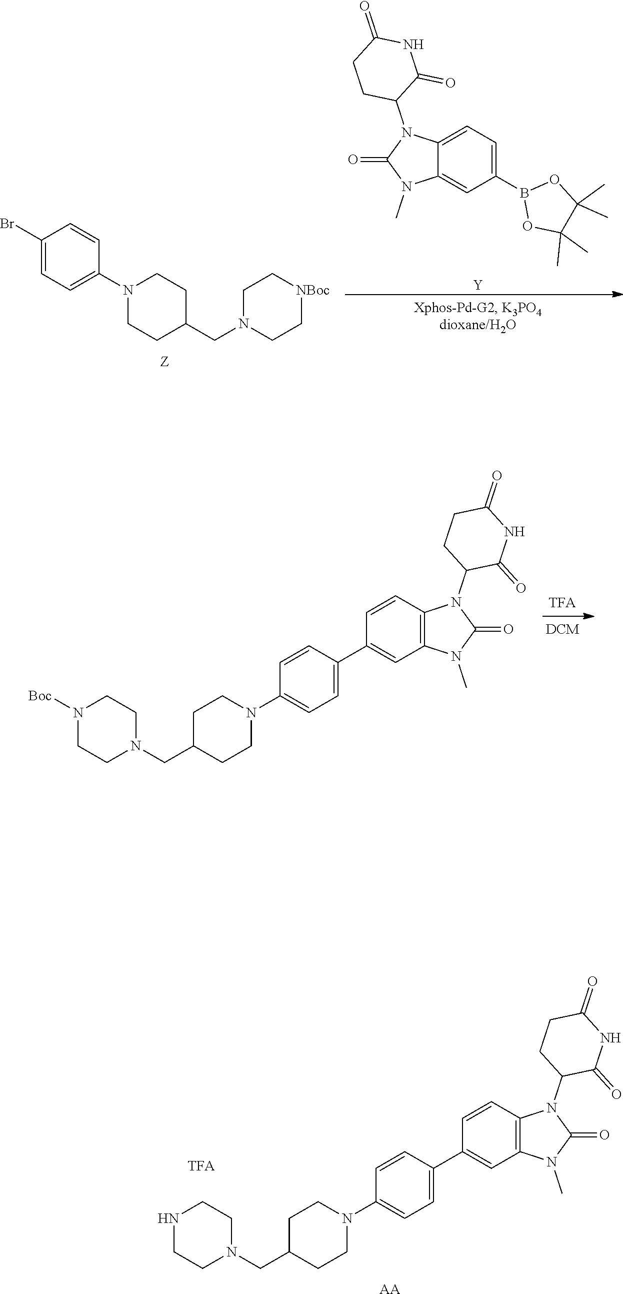

### Step 1—Tert-butyl 4-((1-(4-(1-(2,6-dioxopiperidin-3-yl)-3-methyl-2-oxo-2,3-dihydro-1H-benzo[d]imidazol-5-yl)phenyl)piperidin-4-yl)methyl)piperazine-1-carboxylate

A mixture of tert-butyl 4-((1-(4-bromophenyl)piperidin-4-yl)methyl)piperazine-1-carboxylate (1.9 g, 4.33 mmol, Intermediate Z), 3-(3-methyl-2-oxo-5-(4,4,5,5-tetramethyl-1,3,2-dioxaborolan-2-yl)-2,3-dihydro-1H-benzo[d]imidazol-1-yl)piperidine-2,6-dione (1.67 g, 4.33 mmol, Intermediate Y), chloro(2-dicyclohexylphosphino-2′,4′,6′-triisopropyl-1,1′-biphenyl)[2-(2′-amino-1,1′-biphenyl)]palladium(II) (341 mg, 0.43 mmol) and potassium phosphate (1.84 g, 8.67 mmol) in dioxane (40 mL) and water (4 mL) was degassed and purged with nitrogen three times. Then the mixture was stirred at 80° C. for 12 hours under nitrogen atmosphere. The reaction mixture was diluted with water (30 mL) and extracted with ethyl acetate (40 mL×2). The combined organic layers were washed with brine (50 mL×2), dried over anhydrous sodium sulfate, filtered and concentrated under reduced pressure to give a residue. The crude product was triturated with 30 mL (Petroleum ether:Ethyl acetate=1:2) at 25° C. for 30 minute twice give the title compound (1.35 g, 49% yield) as a gray solid. ¹H NMR (400 MHz, DMSO-d₆) δ=11.11 (s, 1H), 7.54 (d, J=8.8 Hz, 2H), 7.41 (d, J=1.2 Hz, 1H), 7.27 (dd, J=1.2, 8.4 Hz, 1H), 7.15 (d, J=8.2 Hz, 1H), 7.00 (d, J=8.8 Hz, 2H), 5.41 (dd, J=5.4, 12.8 Hz, 1H), 3.73 (d, J=12.4 Hz, 2H), 3.40 (s, 3H), 3.32 (d, J=4.8 Hz, 4H), 2.99-2.87 (m, 1H), 2.80-2.59 (m, 4H), 2.33-2.25 (m, 4H), 2.17 (d, J=7.2 Hz, 2H), 2.09-1.98 (m, 1H), 1.85-1.61 (m, 3H), 1.40 (s, 9H), 1.29-1.14 (m, 2H); LC-MS (ESI⁺) m/z 617.1 (M+1)⁺.

### Step 2—3-(3-Methyl-2-oxo-5-(4-(4-(piperazin-1-ylmethyl)piperidin-1-yl)phenyl)-2,3-dihydro-1H-benzo[d]imidazol-1-yl)piperidine-2,6-dione Trifluoroacetate

To a stirred mixture of tert-butyl 4-[(1-{4-[1-(2,6-dioxopiperidin-3-yl)-3-methyl-2-oxo-1,3-benzodiazol-5-yl]phenyl}piperidin-4-yl)methyl]piperazine-1-carboxylate (75 mg, 0.12 mmol) in DCM (2 mL) was added TFA (0.4 mL) dropwise at rt under N₂ atmosphere. The resulting mixture was stirred for 1 h at rt under N₂ atmosphere. On completion, the reaction mixture was triturated with Et2O (6 mL) to afford the title compound (74 mg) as a white solid. LC/MS (ESI, m/z): [(M+H)]⁺=517.3.

### Example 1.28. Preparation of tert-butyl 4-((1r,4r)-4-(4-(((trifluoromethyl)sulfonyl)oxy)phenyl)cyclohexyl)piperazine-1-carboxylate (Intermediate AB)

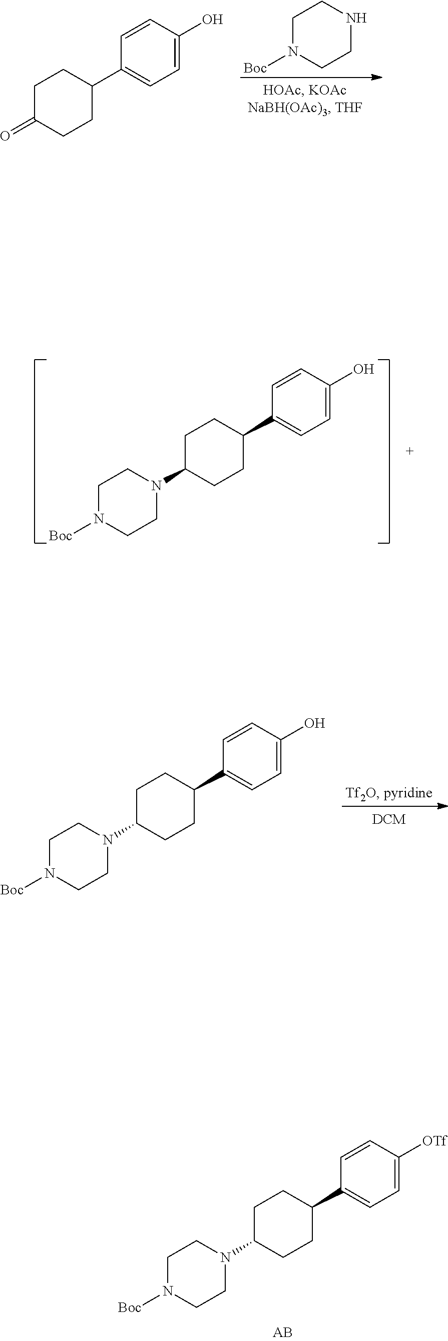

### Step 1—Tert-butyl 4-((1s,4s)-4-(4-hydroxyphenyl)cyclohexyl)piperazine-1-carboxylate and tert-butyl 4-((1r,4r)-4-(4-hydroxyphenyl)cyclohexyl)piperazine-1-carboxylate

To a solution of 4-(4-hydroxyphenyl)cyclohexanone (5 g, 26.2 mmol) and tert-butyl piperazine-1-carboxylate (4.90 g, 26.2 mmol) in THF (100 mL) was added HOAc (4.74 g, 78.8 mmol), KOAc (7.74 g, 78.8 mmol) and 4 Å molecular sieves (4 g), and mixture was stirred at 25° C. for 0.5 h. Then NaBH(OAc)₃ (16.7 g, 78.8 mmol) was added and the mixture was stirred at 25 C for 12 h. On completion, the reaction mixture was the mixture was filtered and the filtrate was concentrated. The residue was purified by prep-HPLC (column: YMC Triart C18 250*50 mm*7 um; mobile phase: [water(NH4HCO3)-ACN]; B %: 40%-70%, 21 min) to afford tert-butyl 4-((1s,4s)-4-(4-hydroxyphenyl)cyclohexyl)piperazine-1-carboxylate (3.5 g, 9.71 mmol) as a white solid (¹H NMR (400 MHz, DMSO-d₆) δ=9.09 (br s, 1H), 7.00 (d, J=8.4 Hz, 2H), 6.66 (d, J=8.4 Hz, 2H), 3.35-3.25 (m, 5H), 2.52 (br d, J=2.0 Hz, 1H), 2.38-2.28 (m, 4H), 2.17 (br s, 1H), 1.89 (br d, J=11.5 Hz, 2H), 1.77 (q, J=11.6 Hz, 2H), 1.55-1.42 (m, 4H), 1.39 (s, 9H)) and tert-butyl 4-((1r,4r)-4-(4-hydroxyphenyl)cyclohexyl)piperazine-1-carboxylate (2.1 g, 5.83 mmol) as a white solid (¹H NMR (400 MHz, DMSO-d₆) δ=9.09 (s, 1H), 6.99 (d, J=8.4 Hz, 2H), 6.65 (d, J=8.4 Hz, 2H), 3.31-3.26 (m, 4H), 2.48-2.43 (m, 5H), 2.38-2.26 (m, 2H), 1.90-1.73 (m, 4H), 1.40 (s, 9H), 1.38-1.30 (m, 4H)).

### Step 2—tert-butyl 4-((1r,4r)-4-(4-(((trifluoromethyl)sulfonyl)oxy)phenyl)cyclohexyl)piperazine-1-carboxylate

To a solution of tert-butyl 4-[4-(4-hydroxyphenyl)cyclohexyl]piperazine-1-carboxylate (2.00 g, 5.55 mmol) in DCM (20 mL) was added pyridine (877 mg, 11.1 mmol) and trifluoromethylsulfonyl trifluoromethanesulfonate (1.88 g, 6.66 mmol) under N₂ at 0° C. The mixture was then stirred at 25° C. for 12 h. On completion, the mixture was diluted with DCM (50 mL), washed with NaHCO₃ (30 mL×3) and brine (30 mL×3) and dried over Na₂SO₄ and concentrated in vacuo to afford the title compound (2.1 g) as a yellow solid. LC-MS (ESI+) m/z 493.1 (M+H)⁺.

### Example 1.29. Preparation of 3-(3-methyl-2-oxo-5-(4-((1r,4r)-4-(piperazin-1-yl)cyclohexyl)phenyl)-2,3-dihydro-1H-benzo[d]imidazol-1-yl)piperidine-2,6-dione (Intermediate AC)

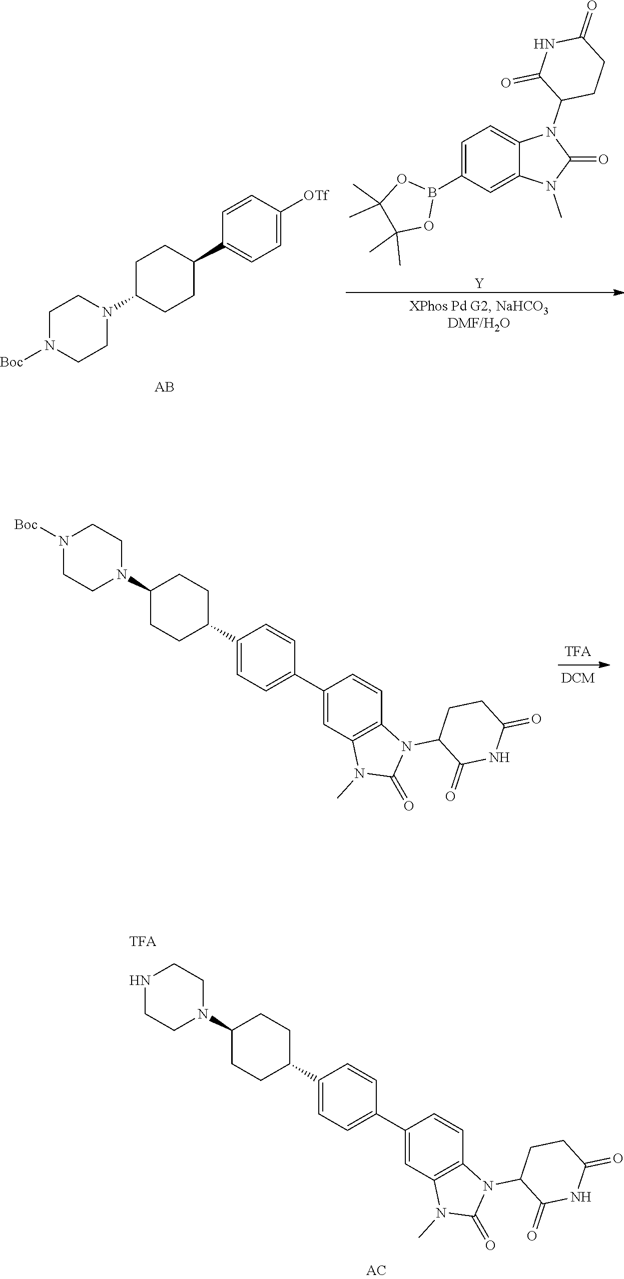

### Step 1—Tert-butyl 4-((1r,4r)-4-(4-(1-(2,6-dioxopiperidin-3-yl)-3-methyl-2-oxo-2,3-dihydro-1H-benzo[d]imidazol-5-yl)phenyl)cyclohexyl)piperazine-1-carboxylate

To a solution of tert-butyl 4-[4-[4-(trifluoromethylsulfonyloxy)phenyl]cyclohexyl]piperazine-1-carboxylate (1.53 g, 3.12 mmol, Intermediate AB) and 3-[3-methyl-2-oxo-5-(4,4,5,5-tetramethyl-1,3,2-dioxaborolan-2-yl)benzimidazol-1-yl]piperidine-2,6-dione (1 g, 2.60 mmol, Intermediate Y) in DMF (20 mL) and H₂O (2 mL) was added XPHOS-PD-G2 (204 mg, 259 umol) and NaHCO₃ (436 mg, 5.19 mmol), then the mixture was stirred at 80° C. for 4 h. On completion, the reaction mixture was filtered and filtered cake was dried. The filter cake was then purified by column chromatography (SiO₂, Petroleum ether/Ethyl acetate=0/1 to EA/DCM=1/1+0.1% TEA) to afford the title compound (1.2 g, 1.99 mmol, 77% yield) as a yellow solid. LC-MS (ESI⁺) m/z 602.3 (M+H)⁺; ¹H NMR (400 MHz, DMSO-d₆) δ=11.11 (s, 1H), 7.59 (d, J=8.1 Hz, 2H), 7.46 (d, J=1.4 Hz, 1H), 7.35-7.23 (m, 3H), 7.17 (d, J=8.3 Hz, 1H), 5.39 (dd, J=5.3, 12.8 Hz, 1H), 3.40 (s, 3H), 3.31-3.26 (m, 4H), 2.98-2.85 (m, 1H), 2.81-2.70 (m, 1H), 2.67-2.60 (m, 1H), 2.39 (br s, 6H), 2.11-2.01 (m, 1H), 1.94-1.82 (m, 4H), 1.58-1.45 (m, 2H), 1.40 (s, 11H).

### Step 2—3-(3-Methyl-2-oxo-5-(4-((1r,4r)-4-(piperazin-1-yl)cyclohexyl)phenyl)-2,3-dihydro-1H-benzo[d]imidazol-1-yl)piperidine-2,6-dione Trifluoroacetate

To a stirred solution of tert-butyl 4-[(1r,4r)-4-{4-[1-(2,6-dioxopiperidin-3-yl)-3-methyl-2-oxo-1,3-benzodiazol-5-yl]phenyl}cyclohexyl]piperazine-1-carboxylate (50.7 mg, 0.084 mmol) in DCM (2 mL) was added TFA (0.4 mL) dropwise at rt. The resulting mixture was stirred for additional 1 h at rt. On completion, the reaction mixture was concentrated under reduced pressure. The residue was triturated with diethyl ether (5 mL) to afford the title compound (50 mg) as an off-white solid. LC/MS (ESI, m/z): [(M+H)]⁺=502.3.

### Example 1.30. Preparation of 3-(3-methyl-2-oxo-5-(4-(1-(piperidin-4-yl)azetidin-3-yl)piperazin-1-yl)-2,3-dihydro-1H-benzo[d]imidazol-1-yl)piperidine-2,6-dione (Intermediate AD) and 3-(5-(4-(Azetidin-3-yl)piperazin-1-yl)-3-methyl-2-oxo-2,3-dihydro-1H-benzo[d]imidazol-1-yl)piperidine-2,6-dione (Intermediate GB)

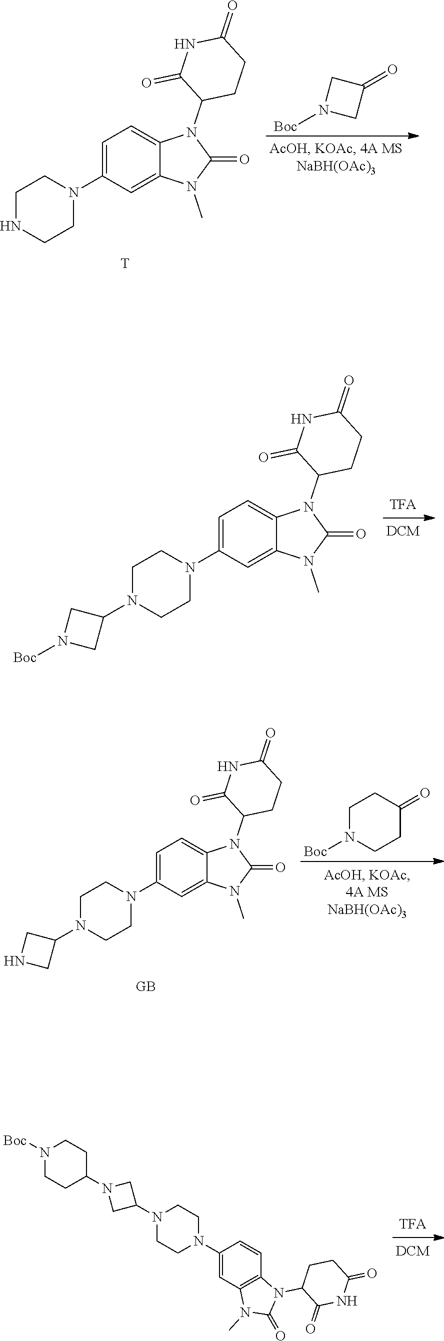

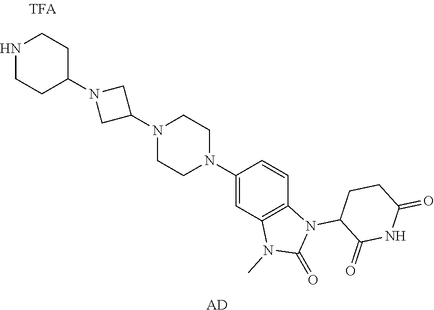 [SMILES: Cn1c(=O)n(C2CCC(=O)NC2=O)c2ccc(N3CCN(C4CN(C5CCNCC5)C4)CC3)cc21 ; "AD"]

### Step 1—Tert-butyl 3-(4-(1-(2,6-dioxopiperidin-3-yl)-3-methyl-2-oxo-2,3-dihydro-1H-benzo[d]imidazol-5-yl)piperazin-1-yl)azetidine-1-carboxylate

To a solution of tert-butyl 3-oxoazetidine-1-carboxylate (538 mg, 3.15 mmol, CAS #398489-26-4) and 3-(3-methyl-2-oxo-5-(piperazin-1-yl)-2,3-dihydro-1H-benzo[d]imidazol-1-yl)piperidine-2,6-dione (1.08 g, 3.15 mmol, Intermediate T) in THF (10 mL) was added KOAc (926 mg, 9.44 mmol), AcOH (567 mg, 9.44 mmol), and 4 Å molecular sieves (0.5 g, 3.15 mmol) at 0° C., and the mixture was stirred at 0° C. for 30 min. Then NaBH(OAc)₃ (2.00 g, 9.44 mmol) was added and the mixture was stirred at 0-20° C. for 12 hr. On completion, the mixture was filtered, the filter liquor was poured into water (10 ml). Then the mixture was extracted with DCM (10 ml×3) washed with brine (10 ml×3), and the organic phase concentrated. The residue was purified by column chromatography (SiO₂, Petroleum ether/Ethyl acetate=1/1-0/1) to give the title compound (972 mg, 62% yield) as a yellow oil. LC-MS (ESI⁺) m/z 499.1 (M+H)⁺.

### Step 2—3-(5-(4-(Azetidin-3-yl)piperazin-1-yl)-3-methyl-2-oxo-2,3-dihydro-1H-benzo[d]imidazol-1-yl)piperidine-2,6-dione

The solution of tert-butyl 3-(4-(1-(2,6-dioxopiperidin-3-yl)-3-methyl-2-oxo-2,3-dihydro-1H-benzo[d]imidazol-5-yl)piperazin-1-yl)azetidine-1-carboxylate (970 mg, 1.95 mmol) in DCM (10 mL) was added TFA (2 mL), then the mixture was stirred at 25° C. for 0.5 h. On completion, the mixture was concentrated under reduced pressure to give the title compound (1.5 g) as a white solid. LC-MS (ESI+) m/z 399.0 (M+H)+.

### Step 3—Tert-butyl 4-(3-(4-(1-(2,6-dioxopiperidin-3-yl)-3-methyl-2-oxo-2,3-dihydro-1H-benzo[d]imidazol-5-yl)piperazin-1-yl)azetidin-1-yl)piperidine-1-carboxylate

To a solution of 3-[5-[4-(azetidin-3-yl)piperazin-1-yl]-3-methyl-2-oxo-benzimidazol-1-yl]piperidine-2,6-dione (1.5 g, 3.76 mmol) in THF (30 mL) was added AcOH (678 mg, 11.3 mmol), KOAc (1.11 g, 11.3 mmol), 4 Å molecular sieves (0.1 g, 3.76 mmol) and tert-butyl 4-oxopiperidine-1-carboxylate (750 mg, 3.76 mmol) and the mixture was stirred at 0° C. for 30 min. Then, NaBH(OAc)₃ (2.39 g, 11.3 mmol) was added and the mixture was stirred at 0-20° C. for 12 h. On completion, the mixture was poured into NH₄Cl solution (20 ml), then the mixture was extracted with DCM (20 ml×3), washed with brine (20 ml×3), and the organic phase concentrated. The residue was purified by prep-HPLC (column: YMC Triart C18 250*50 mm*7 um; mobile phase: [water (FA)-ACN]; B %: 10%-40%, 10 min) to give the title compound (695 mg, 31% yield) as a yellow solid. LC-MS (ESI⁺) m/z 582.3 (M+H)⁺. ¹H NMR (400 MHz, DMSO-d6) δ=11.06 (s, 1H), 6.94 (d, J=8.4 Hz, 1H), 6.85 (d, J=2.0 Hz, 1H), 6.64 (dd, J=2.0, 8.8 Hz, 1H), 5.34-5.22 (m, 1H), 4.06-3.84 (m, 4H), 3.77-3.61 (m, 2H), 3.31 (br s, 3H), 3.12 (br s, 5H), 2.95-2.84 (m, 1H), 2.81-2.56 (m, 5H), 2.56-2.51 (m, 4H), 2.03 (s, 1H), 1.86-1.75 (m, 2H), 1.39 (s, 9H), 1.20-1.06 (m, 2H)

### Step 4—3-(3-Methyl-2-oxo-5-(4-(1-(piperidin-4-yl)azetidin-3-yl)piperazin-1-yl)-2,3-dihydro-1H-benzo[d]imidazol-1-yl)piperidine-2,6-dione Trifluoroacetate

To a stirred solution of tert-butyl 4-(3-{4-[1-(2,6-dioxopiperidin-3-yl)-3-methyl-2-oxo-1,3-benzodiazol-5-yl]piperazin-1-yl}azetidin-1-yl)piperidine-1-carboxylate (50.38 mg, 0.087 mmol) in DCM (2 mL) was added TFA (0.4 mL) dropwise at rt. The resulting mixture was stirred for additional 1 h at rt. On completion, the reaction was concentrated under reduced pressure. The residue was triturated with diethyl ether (5 mL) to afford the title compound (49 mg, 98%) as a yellow solid. LC/MS (ESI, m/z): [(M+H)]⁺=482.3.

### Example 1.31. Preparation of 3-(3-Methyl-2-oxo-4-(2,7-diazaspiro[3.5]nonan-7-yl)-2,3-dihydro-1H-benzo[d]imidazol-1-yl)piperidine-2,6-dione (Intermediate AE)

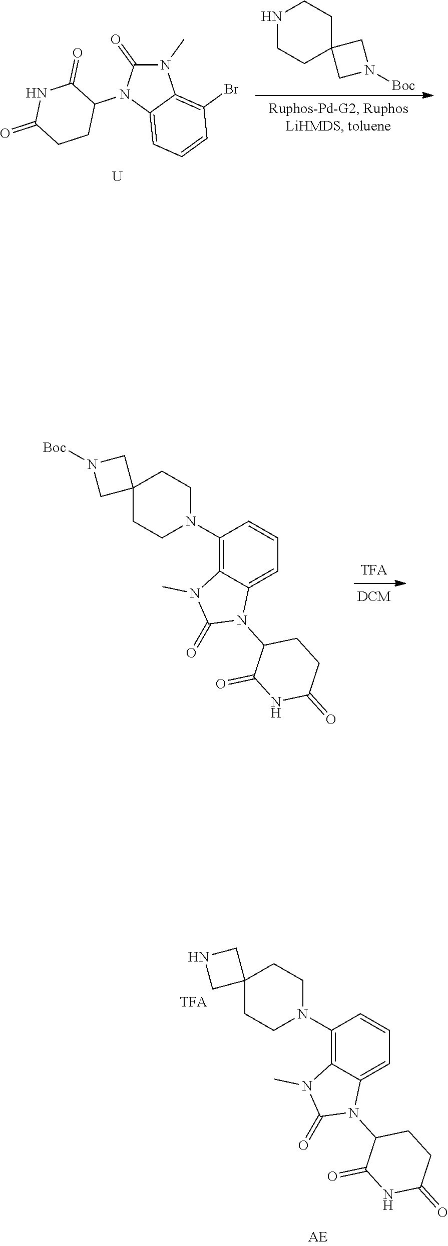

### Step 1—Tert-butyl 7-(1-(2,6-dioxopiperidin-3-yl)-3-methyl-2-oxo-2,3-dihydro-1H-benzo[d]imidazol-5-yl)-2,7-diazaspiro[3.5]nonane-2-carboxylate

To a solution of 3-(4-bromo-3-methyl-2-oxo-benzimidazol-1-yl)piperidine-2,6-dione (6 g, 20 mmol, Intermediate U), tert-butyl 2,7-diazaspiro[3.5]nonane-2-carboxylate (4.82 g, 21.3 mmol, CAS #236406-55-6), [2-(2-aminophenyl)phenyl]-chloro-palladium; dicyclohexyl-[2-(2, 6-diisopropoxyphenyl)phenyl]phosphane (2.76 g, 3.55 mmol), LiHMDS (1 M, 88.72 mL) and 4 Å molecular sieves (1.2 g) in toluene (120 mL) was added RuPhos (1.66 g, 3.55 mmol). The mixture was stirred at 80° C. under N₂ for 2 hr. On completion, the mixture was adjusted to pH=5 with FA at 0° C., then filtered, and the filtrate was concentrated under reduced pressure to give a residue. The residue was purified by column chromatography (SiO₂, PE:EA=2:1 to 0:1, P: Rf=0.4 (PE:EA=0:1)) to give the title compound (4.6 g, 50% yield) as off-white solid. ¹H NMR (400 MHz, CDCl₃-d) δ=8.11 (s, 1H), 7.02-6.98 (m, 1H), 6.90-6.88 (d, J=8.0 Hz, 1H), 6.59-6.57 (m, 1H), 5.23-5.19 (dd, J=5.2, 12.8 Hz, 1H), 3.76 (s, 5H), 3.71-3.64 (m, 2H), 3.11-3.08 (m, 2H), 2.93-2.92 (m, 1H), 2.82-2.69 (m, 4H), 2.24-2.22 (m, 1H), 1.95-1.92 (m, 4H), 1.47 (s, 9H).

### Step 2—3-(3-methyl-2-oxo-4-(2,7-diazaspiro[3.5]nonan-7-yl)-2,3-dihydro-1H-benzo[d]imidazol-1-yl)piperidine-2,6-dione Trifluoroacetate

To a stirred mixture of tert-butyl 7-[1-(2,6-dioxopiperidin-3-yl)-3-methyl-2-oxo-1,3-benzodiazol-4-yl]-2,7-diazaspiro[3.5]nonane-2-carboxylate (50 mg, 0.103 mmol) in DCM (2 mL) was added TFA (0.4 mL) dropwise at room temperature. The resulting mixture was stirred for 1 h at room temperature under N₂ atmosphere. On completion, the residue was triturated with Et₂O (5 mL) to give the title compound (49 mg) as a white solid. LC/MS (ESI, m/z): [(M+H)]⁺=384.2.

### Example 1.32. Preparation of 6-((5-((3-fluoro-2-methoxyphenyl)amino)-6-((methyl-d₃)carbamoyl)pyridazin-3-yl)amino)nicotinic Acid (Intermediate AF)

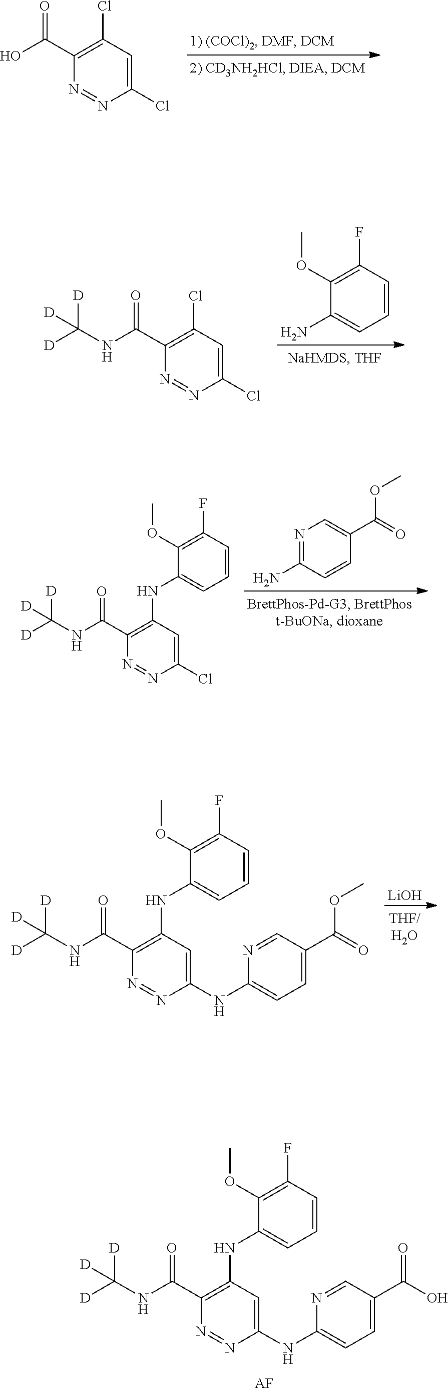

### Step 1—4,6-Dichloro-N-(methyl-d₃)pyridazine-3-carboxamide

To a stirred mixture of 4,6-dichloropyridazine-3-carboxylic acid (1 g, 5 mmol) in DCM (15 mL) were added (COCl)₂ (0.66 mL, 7.8 mmol) and DMF (37.88 mg, 0.518 mmol) in turns at 0° C. under N₂ atmosphere. The resulting mixture was stirred for 1 h at rt under air atmosphere. On completion, the reaction mixture was concentrated under reduced pressure. The residue was re-dissolved DCM (15 mL) and marked as solution A. Then solution A was added a solution of Methan-d₃-amine hydrochloride (0.70 g, 10.364 mmol) and DIEA (1.34 g, 10.364 mmol) in DCM (10 mL) dropwise over 5 min at 0° C. The resulting mixture was stirred for additional 1 h at rt under N₂ atmosphere. On completion, the reaction mixture was extracted with DCM (4×100 mL). The combined organic layer was washed with brine (2×100 mL) and dried over anhydrous Na₂SO₄. After filtration, the filtrate was concentrated under reduced pressure. The residue was purified by silica gel column chromatography, eluted with PE/EA (1:1) to afford the title compound (1 g, 92% yield) as a white solid. ¹H NMR (400 MHz, DMSO-d₆) δ 8.89 (s, 1H), 8.49 (d, J=0.9 Hz, 1H); LC/MS (ESI, m/z): [(M+1)]⁺=209.0.

### Step 2—6-Chloro-4-((3-fluoro-2-methoxyphenyl)amino)-N-(methyl-d₃)pyridazine-3-carboxamide

To a stirred mixture of 4,6-dichloro-N-(methyl-d₃)pyridazine-3-carboxamide (0.81 g, 5.7 mmol) in THF (15 mL) was added NaHMDS (6 mL, 12 mmol) dropwise at rt under N₂ atmosphere. The resulting mixture was stirred for 2 h at rt under air atmosphere. On completion, the reaction mixture was concentrated under vacuum. The residue was purified by reverse phase flash chromatography (Column: WelFlash™ C18-I, 20-40 um, 330 g; Eluent A: Water (plus 10 mmol/L FA); Eluent B: ACN; Gradient: 25%-55% B in 25 min; Flow rate: 80 mL/min; Detector: 220/254 nm; desired fractions were collected at 50% B) and concentrated under reduced pressure to afford the title compound (800 mg, 53% yield) as a yellow oil. ¹H NMR (400 MHz, Chloroform-d) δ 10.92 (s, 1H), 8.26 (s, 1H), 7.13-7.04 (m, 3H), 7.04-7.00 (m, 2H), 3.97 (d, J=2.1 Hz, 3H); LC/MS (ESI, m/z): [(M+1)]⁺=314.1.

### Step 3—Methyl 6-((5-((3-fluoro-2-methoxyphenyl)amino)-6-((methyl-d₃)carbamoyl)pyridazin-3-yl)amino)nicotinate

To a stirred mixture of 6-chloro-4-((3-fluoro-2-methoxyphenyl)amino)-N-(methyl-d₃)pyridazine-3-carboxamide (1 g, 3 mmol) and methyl 6-aminopyridine-3-carboxylate (0.73 g, 4.8 mmol) in dioxane (20 mL) were added BrettPhos (0.17 g, 0.32 mmol), BrettPhos Pd G3 (0.29 g, 0.32 mmol) and t-BuONa (0.61 g, 6.4 mmol) in turns at rt under N₂ atmosphere. The resulting mixture was stirred for 2 h at 100° C. under N₂ atmosphere. On completion, the mixture was cooled to rt and extracted with EtOAc (3×100 mL) and dried over anhydrous Na₂SO₄. After filtration, the filtrate was concentrated under reduced pressure. The residue was purified by reverse phase flash chromatography (Column: WelFlash™ C18-I, 20-40 um, 330 g; Eluent A: Water (plus 10 mmol/L FA); Eluent B: ACN; Gradient: 25%-55% B in 25 min; Flow rate: 80 mL/min; Detector: 220/254 nm; desired fractions were collected at 45% B) and concentrated under reduced pressure to afford the title compound (800 mg, 59% yield) as a yellow solid. ¹H NMR (400 MHz, DMSO-d₆) δ 11.04 (s, 1H), 10.71 (s, 1H), 9.17 (s, 1H), 8.74 (d, J=2.4 Hz, 1H), 8.19 (dd, J=8.9, 2.4 Hz, 1H), 8.15 (s, 1H), 7.76 (d, J=8.8 Hz, 1H), 7.44-7.40 (m, 1H), 7.29-7.19 (m, 1H), 7.13-7.05 (m, 2H), 3.88 (s, 3H), 3.85 (s, 3H); LC/MS (ESI, m/z): [(M+1)]⁺=430.1.

### Example 1.33. Preparation of 6-((5-((3-fluoro-2-methoxyphenyl)amino)-6-((methyl-d₃)carbamoyl)pyridazin-3-yl)amino)nicotinic Acid

To a stirred mixture of methyl 6-((5-((3-fluoro-2-methoxyphenyl)amino)-6-((methyl-d₃)carbamoyl)pyridazin-3-yl)amino)nicotinate (800 mg, 2 mmol) and LiOH (447.36 mg, 18.64 mmol) in THF (1 mL) and H₂O (1 mL) at rt. The resulting mixture was stirred for 1 h at 50° C. under N₂ atmosphere. On completion, the reaction mixture was concentrated and the residue was purified by reverse phase flash chromatography (Column: WelFlash™ C18-I, 20-40 um, 40 g; Eluent A: Water (plus 10 mmol/L FA); Eluent B: ACN; Gradient: 25%-55% B in 25 min; Flow rate: 40 mL/min; Detector: 220/254 nm; desired fractions were collected at 53% B) and concentrated under reduced pressure to afford the title compound (550 mg, 71% yield) as a white solid. ¹H NMR (400 MHz, DMSO-d₆) δ 11.01 (s, 1H), 10.42 (s, 1H), 9.13 (s, 1H), 8.66 (s, 1H), 8.26 (s, 1H), 8.15-8.08 (m, 1H), 7.56 (d, J=8.7 Hz, 1H), 7.43 (d, J=8.3 Hz, 1H), 7.26-7.20 (m, 1H), 7.14-7.04 (m, 1H), 3.88 (s, 3H); LC/MS (ESI, m/z): [(M+1)]⁺=416.1.

### Example 1.34. Preparation of N-[(1R,2S)-2-fluorocyclopropyl]-6-{[3-(5-formylpyridin-2-yl)phenyl]amino}-8-(methylamino)imidazo[1,2-b]pyridazine-3-carboxamide (Intermediate AG)

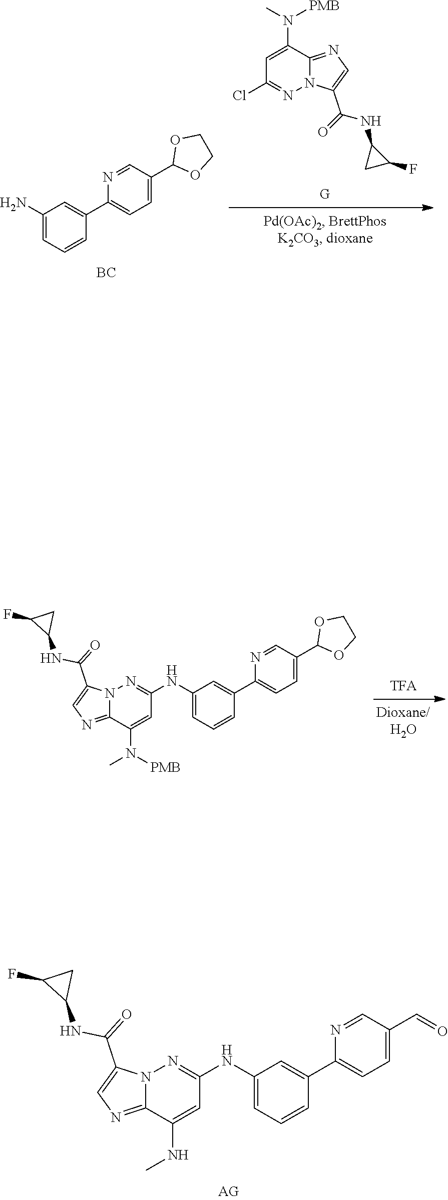

### Step 1—6-({3-[5-(1,3-dioxolan-2-yl)pyridin-2-yl]phenyl}amino)-N-[(1R,2S)-2-fluorocyclopropyl]-8-{[(4-methoxyphenyl)methyl](methyl)amino}imidazo[1,2-b]pyridazine-3-carboxamide

To a stirred mixture of 3-[5-(1,3-dioxolan-2-yl)pyridin-2-yl]aniline (3 g, 12 mmol, Intermediate BC) and 6-chloro-N-[(1R,2S)-2-fluorocyclopropyl]-8-{[(4-methoxyphenyl)methyl](methyl)amino}imidazo[1,2-b]pyridazine-3-carboxamide (6.00 g, 14.9 mmol, Intermediate G) in 1,4-dioxane (30 mL) were added K₂CO₃ (3.42 g, 24.8 mmol), Pd(OAc)₂ (0.28 g, 1.2 mmol) and BrettPhos (0.66 g, 1.2 mmol) in turns at rt under nitrogen atmosphere. The resulting mixture was stirred at reflux overnight. On completion, the reaction mixture was cooled to rt and the mixture was filtered. The filter cake was washed with 1,4-dioxane (5×20 mL). The filtrate was concentrated under reduced pressure. The residue was purified by trituration with CH₂Cl₂ (200 mL). The precipitated solids were collected by filtration and washed with CH₂Cl₂ (5×3 mL) and dried under reduced pressure to give the title compound (7 g, 93% yield) as a brown solid. ¹H NMR (400 MHz, Chloroform-d) δ 9.17 (d, J=4.2 Hz, 1H), 8.77 (d, J=2.2 Hz, 1H), 8.17 (s, 1H), 8.01-7.99 (m, 1H), 7.90-7.86 (m, 1H), 7.78-7.74 (m, 1H), 7.74-7.69 (m, 1H), 7.46 (t, J=7.8 Hz, 1H), 7.43-7.37 (m, 1H), 7.20-7.14 (m, 2H), 6.88-6.84 (m, 2H), 6.46 (s, 1H), 5.91 (s, 1H), 5.39 (s, 2H), 4.73 (d, J=2.8 Hz, 1H), 4.67-4.47 (m, 1H), 4.20-4.14 (m, 2H), 4.12-4.07 (m, 2H), 3.80 (s, 3H), 3.18 (s, 3H), 2.96-2.92 (m, 1H), 1.08-0.98 (m, 1H), 0.88-0.77 (m, 1H). LC/MS (ESI, m/z): [(M+H)]⁺=610.3.

### Step 2—6-chloro-N-[(1R,2S)-2-fluorocyclopropyl]-8-{[(4-methoxyphenyl) methyl] (methyl) amino}imidazo[1,2-b]pyridazine-3-carboxamide

To a stirred mixture of 6-({3-[5-(1,3-dioxolan-2-yl)pyridin-2-yl]phenyl}amino)-N-[(1R,2S)-2-fluorocyclopropyl]-8-{[(4-methoxyphenyl)methyl](methyl)amino}imidazo[1,2-b]pyridazine-3-carboxamide (3.2 g, 5.3 mmol) in 1,4-dioxane (30 mL) and H₂O (10 mL) was added and TFA (10 mL) dropwise at rt under nitrogen atmosphere. The resulting mixture was stirred for overnight at 50° C. under nitrogen atmosphere. On completion, the reaction mixture was cooled to rt and concentrated under reduced pressure. The residue was purified by trituration with water (200 mL). The precipitated solids were collected by filtration and washed with water (2×20 mL). The collected solid was further purified by trituration with EtOAc (100 mL). The resulting solid was dried under reduced pressure to give the title compound (1.5 g, 64% yield) as an orange solid. ¹H NMR (400 MHz, DMSO-d₆) δ 10.14 (s, 1H), 9.33 (s, 1H), 9.16 (d, J=2.1 Hz, 1H), 8.79 (d, J=4.0 Hz, 1H), 8.39-8.27 (m, 2H), 8.23 (d, J=8.3 Hz, 1H), 7.89 (s, 1H), 7.84 (d, J=7.7 Hz, 1H), 7.61 (dd, J=7.9, 2.2 Hz, 1H), 7.57-7.46 (m, 2H), 5.82 (s, 1H), 4.67-4.47 (m, 1H), 2.90 (d, J=4.5 Hz, 3H), 2.76-2.71 (m, 1H), 0.92-0.83 (m, 1H), 0.64-0.53 (m, 1H); LC/MS (ESI, m/z): [(M+H)]⁺=446.2.

### Example 1.35. Preparation of tert-butyl 4-(4-(4,4,5,5-tetramethyl-1,3,2-dioxaborolan-2-yl)phenyl)piperazine-1-carboxylate (Intermediate AH)

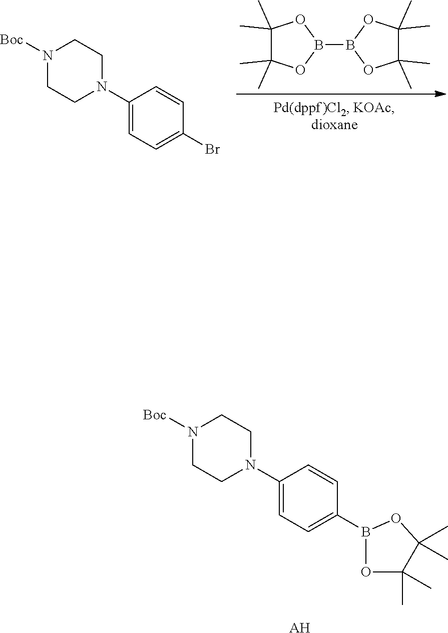

To a stirred mixture of tert-butyl 4-(4-bromophenyl)piperazine-1-carboxylate (1 g, 3 mmol, CAS #352437-09-3) and bis(pinacolato)diboron (1.49 g, 5.86 mmol) in 1,4-dioxane (30 mL) were added Pd(dppf)Cl₂ (0.21 g, 0.29 mmol) and KOAc (0.58 g, 5.9 mmol) at rt under nitrogen atmosphere. The resulting mixture was stirred for 1 h at 90° C. under nitrogen atmosphere. On completion, the reaction mixture was cooled to rt and concentrated under reduced pressure. The residue was purified by silica gel column chromatography, eluted with PE/EA (5:1), to afford the title compound (980 mg, 86% yield) as an off-white solid. ¹H NMR (400 MHz, Chloroform-d) δ 7.85-7.70 (m, 2H), 7.01-6.89 (m, 2H), 3.77-3.51 (m, 4H), 3.38-3.10 (m, 4H), 1.50 (s, 9H), 1.35 (s, 12H). LC/MS (ESI, m/z): [(M+H)]⁺=389.2.

### Example 1.36. Preparation of 3-(3-Methyl-2-oxo-5-(4-(piperazin-1-yl)phenyl)-2,3-dihydro-1H-benzo[d]imidazol-1-yl)piperidine-2,6-dione (Intermediate AI)

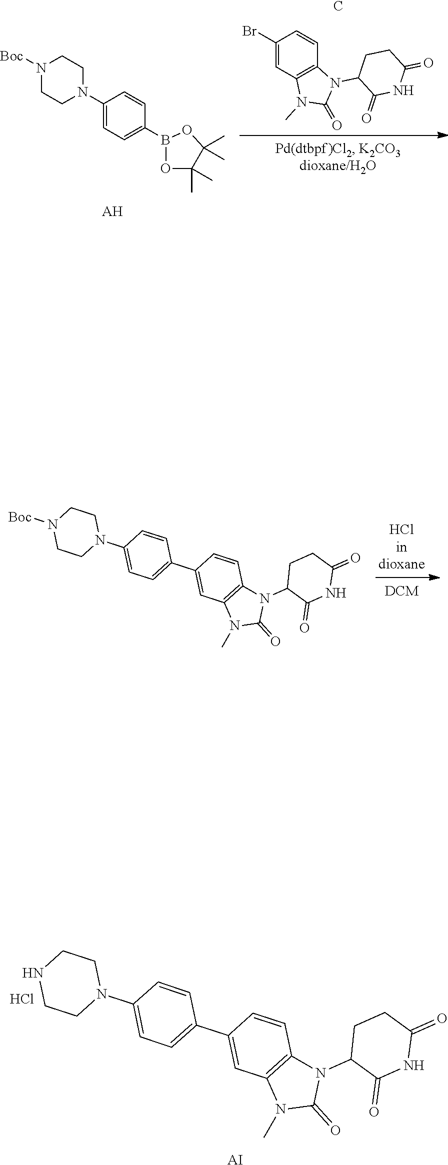

### Step 1—Tert-butyl 4-(4-(1-(2,6-dioxopiperidin-3-yl)-3-methyl-2-oxo-2,3-dihydro-1H-benzo[d]imidazol-5-yl)phenyl)piperazine-1-carboxylate

To a stirred mixture of 3-(5-bromo-3-methyl-2-oxo-1,3-benzodiazol-1-yl)piperidine-2,6-dione (500 mg, 1.5 mmol, Intermediate C) and tert-butyl 4-[4-(4,4,5,5-tetramethyl-1,3,2-dioxaborolan-2-yl)phenyl]piperazine-1-carboxylate (861.25 mg, 2.219 mmol, Intermediate AH) in 1,4-dioxane (8 mL) and water (1.6 mL) were added Pd(dtbpf)Cl₂ (96.37 mg, 0.148 mmol) and K₂CO₃ (408.70 mg, 2.958 mmol) at rt under nitrogen atmosphere. The resulting mixture was stirred for 1 h at 80° C. under nitrogen atmosphere. On completion, the reaction mixture cooled to rt and acidified to pH 3 with HCl (aq. 2 mol/L). The resulting mixture was concentrated under reduced pressure. The residue was purified by reverse phase flash chromatography (Column: WelFlash™ C18-I, 20-40 um, 120 g; Eluent A: Water (plus 10 mmol/L FA); Eluent B: ACN; Gradient: 20%-50% B in 30 min; Flow rate: 80 mL/min; Detector: 220/254 nm; desired fractions were collected at 35% B) and concentrated under reduced pressure to afford the title compound (210 mg, 27% yield) as a light yellow solid. ¹H NMR (400 MHz, DMSO-d₆) δ 11.11 (s, 1H), 7.63-7.52 (m, 2H), 7.46-7.40 (m, 1H), 7.34-7.25 (m, 1H), 7.18-7.11 (m, 1H), 7.07-7.00 (m, 2H), 5.43-5.34 (m, 1H), 3.54-3.45 (m, 4H), 3.39 (s, 3H), 3.19-3.10 (m, 4H), 2.99-2.86 (m, 2H), 2.83-2.58 (m, 2H), 1.43 (s, 9H). LC/MS (ESI, m/z): [(M+H)]⁺=520.2.

### Step 2—3-(3-Methyl-2-oxo-5-(4-(piperazin-1-yl)phenyl)-2,3-dihydro-1H-benzo[d]imidazol-1-yl)piperidine-2,6-dione Hydrochloride

To a stirred mixture of tert-butyl 4-{4-[1-(2,6-dioxopiperidin-3-yl)-3-methyl-2-oxo-1,3-benzodiazol-5-yl]phenyl}piperazine-1-carboxylate (210 mg, 0.404 mmol) in DCM (6 mL) was added a solution of 4 M HCl (gas) in 1,4-dioxane (1.2 mL) at rt under nitrogen atmosphere. The resulting mixture was stirred for additional 1 h at rt. On completion, the reaction mixture was concentrated under reduced pressure to afford the title compound (180 mg) as a light yellow solid. ¹H NMR (400 MHz, DMSO-d₆) δ 11.11 (s, 1H), 9.33-9.28 (m, 2H), 7.65-7.57 (m, 2H), 7.48-7.42 (m, 1H), 7.30 (d, J=8.2, 1H), 7.16 (d, J=8.2 Hz, 1H), 7.11-7.07 (m, 2H), 5.41 (dd, J=12.8, 5.3 Hz, 1H), 3.57 (s, 3H), 3.46-3.41 (m, 4H), 3.27-3.22 (m, 4H), 2.91-2.89 (m, 1H), 2.77-2.73 (m, 1H), 2.70-2.57 (m, 1H), 2.07-2.04 (m, 1H); LC/MS (ESI, m/z): [(M+H)]⁺=420.2.

### Example 1.37. Preparation of Tert-butyl 4-(piperidin-4-yl)piperazine-1-carboxylate (Intermediate AJ)

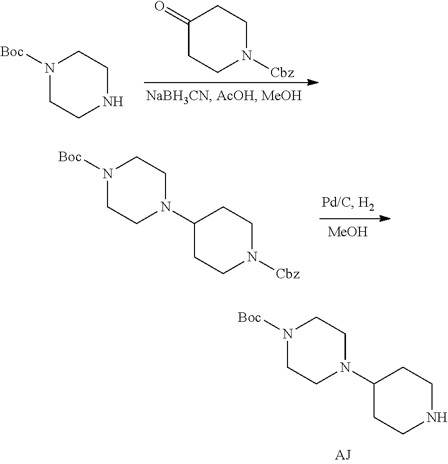

### Step 1—Tert-butyl 4-(1-((benzyloxy)carbonyl)piperidin-4-yl)piperazine-1-carboxylate

To a stirred solution of tert-butyl piperazine-1-carboxylate (1 g, 5 mmol) and benzyl 4-oxopiperidine-1-carboxylate (1.50 g, 6.44 mmol) in MeOH (15 mL) were added NaBH₃CN (0.67 g, 11 mmol) and AcOH (0.32 g, 5.2 mmol) in turns at rt. The resulting mixture was stirred under nitrogen atmosphere at rt for 16 h. On completion, the reaction mixture was concentrated under reduced pressure. The residue was purified by reverse flash chromatography (C18 silica gel; mobile phase, MeCN in Water (10 mmol/L NH₄HCO₃), 30% to 70% gradient in 30 min; detector, UV 254 nm) to afford the title compound (1.4 g, 65% yield) as brown oil. ¹H NMR (400 MHz, Chloroform-d) δ 7.41-7.27 (m, 5H), 5.14 (s, 2H), 4.33-4.17 (m, 2H), 3.52-3.37 (m, 4H), 2.84-2.78 (m, 2H), 2.56-2.47 (m, 4H), 2.48-2.41 (m, 1H), 1.90-1.75 (m, 2H), 1.49-1.45 (m, 11H). LC/MS (ESI, m/z): [(M+H)]⁺=404.2.

### Step 2—Tert-butyl 4-(piperidin-4-yl)piperazine-1-carboxylate

To a solution of tert-butyl 4-{1-[(benzyloxy)carbonyl]piperidin-4-yl}piperazine-1-carboxylate (1.4 g, 3.5 mmol) in MeOH (20 mL) was added Pd/C (0.04 g, 0.347 mmol, 10 wt %) under N₂ atmosphere. The reaction system was degassed under vacuum and purged with H₂ several times, then the mixture was hydrogenated under H₂ balloon (1 atm) at 25° C. for 16 h. After completion of the reaction, Pd/C was filtered off through celite and the corresponding filtrate was concentrated under reduced pressure to provide crude. The crude was purified by silica gel column chromatography, eluted with PE/EA (1:1), to afford the title compound (420 mg, 45% yield) as an off-white solid. ¹H NMR (400 MHz, Chloroform-d) δ 3.53-3.37 (m, 4H), 3.28-3.13 (m, 2H), 2.73-2.56 (m, 2H), 2.55-2.49 (m, 4H), 2.45-2.30 (m, 2H), 1.95-1.74 (m, 2H), 1.50-1.44 (m, 11H). LC/MS (ESI, m/z): [(M+H)]⁺=270.2.

### Example 1.38. Preparation of 3-(3-methyl-2-oxo-5-(4-(piperazin-1-yl)piperidin-1-yl)-2,3-dihydro-1H-benzo[d]imidazol-1-yl)piperidine-2,6-dione (Intermediate AK)

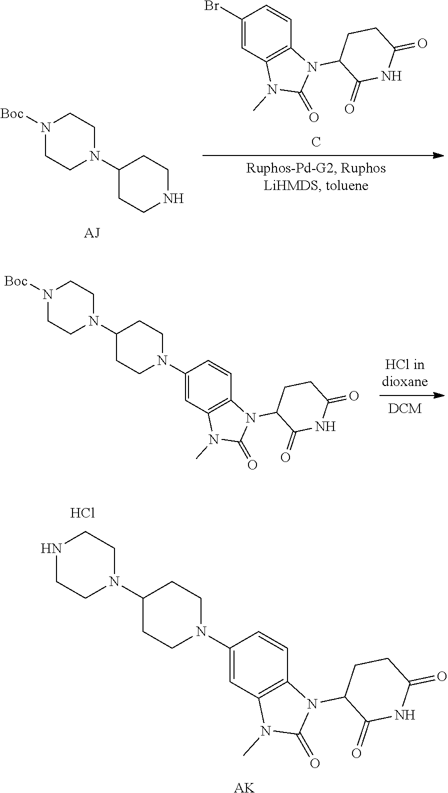

### Step 1—Tert-butyl 4-(1-(1-(2,6-dioxopiperidin-3-yl)-3-methyl-2-oxo-2,3-dihydro-1H-benzo[d]imidazol-5-yl)piperidin-4-yl)piperazine-1-carboxylate

To a stirred solution of 3-(5-bromo-3-methyl-2-oxo-1,3-benzodiazol-1-yl)piperidine-2,6-dione (420 mg, 1.24 mmol, Intermediate C) and tert-butyl 4-(piperidin-4-yl)piperazine-1-carboxylate (501.88 mg, 1.863 mmol, Intermediate AJ) in toluene (6 mL) were added RuPhos-PdCl-2nd G (193.19 mg, 0.248 mmol), RuPhos (115.92 mg, 0.248 mmol) followed by addition dropwise LiHMDS (7.45 mL, 7.45 mmol) within 10 min at nitrogen atmosphere. The resulting mixture was stirred under nitrogen atmosphere at 80° C. for 2 h. On completion, the reaction mixture was cooled to rt and acidified to pH 3 with HCl (aq.2 mol/L). The resulting mixture was concentrated under reduced pressure. The residue was dissolved in DMSO (20 mL) and purified by reverse flash chromatography (Column, C18 silica gel; mobile phase, MeCN in Water (0.1% FA), 20% to 50% gradient in 30 min; detector, UV 254 nm) to afford the title compound (340 mg, 52%) as a brown yellow solid. ¹H NMR (400 MHz, Chloroform-d) δ 8.64 (s, 1H), 6.79-6.59 (m, 3H), 5.42-5.00 (m, 1H), 3.71-3.59 (m, 3H), 3.58-3.47 (m, 4H), 3.52-3.26 (m, 3H), 2.99-2.79 (m, 2H), 2.79-2.48 (m, 7H), 2.31-2.13 (m, 1H), 2.04-1.90 (m, 2H), 1.86-1.68 (m, 2H), 1.48 (s, 9H). LC/MS (ESI, m/z): [(M+H)]⁺=527.3.

### Step 2—3-(3-methyl-2-oxo-5-(4-(piperazin-1-yl)piperidin-1-yl)-2,3-dihydro-1H-benzo[d]imidazol-1-yl)piperidine-2,6-dione

To a stirred solution of tert-butyl 4-{1-[1-(2,6-dioxopiperidin-3-yl)-3-methyl-2-oxo-1,3-benzodiazol-5-yl]piperidin-4-yl}piperazine-1-carboxylate (300 mg, 0.57 mmol) in DCM (6 mL) was added a solution of 4 M HCl (gas) in 1,4-dioxane (3 mL) dropwise at rt. The resulting mixture was stirred for additional 1 h at rt. On completion, the reaction mixture was concentrated under reduced pressure. The residue was triturated with Et₂O (5 mL) to afford the title compound (260 mg) as a light brown solid. LC/MS (ESI, m/z): [(M+H)]⁺=427.2.

### Example 1.39. Preparation of tert-butyl (14-bromo-3,6,9,12-tetraoxatetradecyl) carbamate (Intermediate AL)

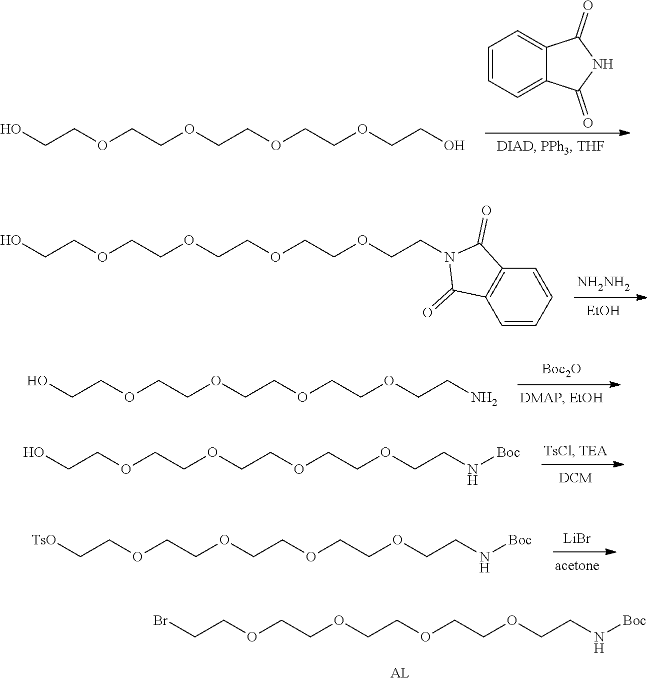

### Step 1—2-(14-Hydroxy-3,6,9,12-tetraoxatetradecyl)isoindoline-1,3-dione

A solution of 2-[2-[2-[2-(2-hydroxyethoxy)ethoxy]ethoxy]ethoxy]ethanol (50 g, 200 mmol, CAS #4792-15-8), isoindoline-1,3-dione (37.05 g, 251.8 mmol) and PPh₃ (82.56 g, 314.8 mmol) in THF (500 mL) was added DIAD (63.65 g, 314.8 mmol, 61.20 mL) at 0° C. and stirred at 20° C. for 2 hr. On completion, the reaction mixture was diluted with H₂O (400 mL) and extracted with EA (300 mL×3). The combined organic layers were washed with brine (100 mL), dried over Na₂SO₄, filtered and concentrated under reduced pressure to give a residue. The residue was purified by reversed-phase HPLC (0.1% FA condition) to give the title compound (40 g, 52% yield) as white solid. ¹H NMR (400 MHz, CDCl₃-d) δ=7.86 (dd, J=3.1, 5.4 Hz, 2H), 7.73 (dd, J=3.0, 5.4 Hz, 2H), 3.94-3.88 (m, 2H), 3.77-3.73 (m, 4H), 3.69-3.60 (m, 14H).

### Step 2—14-Amino-3,6,9,12-tetraoxatetradecan-1-ol

To a solution of 2-[2-[2-[2-[2-(2-hydroxyethoxy) ethoxy] ethoxy] ethoxy]ethyl]isoindoline-1,3-dione (35 g, 95 mmol) in EtOH (500 mL) was added NH₂NH₂·H₂O (9.54 g, 190 mmol, 9.26 mL). The mixture was stirred at 80° C. for 2 hr. On completion, the reaction was filtered to remove the insoluble materials. The filtrate was concentrated in vacuo to give the title compound (22 g) was obtained as white solid.

### Step 3—Tert-butyl (14-hydroxy-3,6,9,12-tetraoxatetradecyl)carbamate

To a solution of 2-[2-[2-[2-(2-aminoethoxy)ethoxy]ethoxy]ethoxy]ethanol (22 g, 93 mmol) in EtOH (300 mL) was added (Boc)₂O (40.47 g, 185.4 mmol, 42.60 mL) and DMAP (3.40 g, 27.8 mmol). The mixture was stirred at 25° C. for 16 h. On completion, the reaction mixture was filtered to remove the insoluble materials. The filtrate was concentrated in vacuo. The residue was purified by column chromatography (SiO₂, PE:EA=1:1 to 0:1, P: Rf=0.1) to give the title compound (20 g, 59 mmol, 64% yield) as colorless oil. ¹H NMR (400 MHz, CHLOROFORM-d) δ=3.78-3.74 (m, 2H), 3.71-3.62 (m, 14H), 3.56 (t, J=5.1 Hz, 2H), 3.33 (t, J=5.1 Hz, 2H), 1.46 (s, 9H).

### Step 4—Tert-butyl (14-hydroxy-3,6,9,12-tetraoxatetradecyl)carbamate

To a solution of tert-butyl N-[2-[2-[2-[2-(2-hydroxyethoxy)ethoxy]ethoxy]ethoxy]ethyl]carbamate (23 g, 68 mmol), DMAP (832.78 mg, 6.82 mmol) and TEA (20.69 g, 204.5 mmol, 28.46 mL) in DCM (250 mL) was added TosCl (25.99 g, 136.33 mmol) at 0° C. The mixture was stirred at 25° C. for 12 hr. On completion, the reaction mixture was concentrated under reduced pressure to give a residue. The residue was purified by column chromatography (SiO₂, PE:EA=3:1 to 0:1, P: Rf=0.6) to give the title compound (28 g, 84% yield) as yellow oil.

### Step 5—Tert-butyl (14-bromo-3,6,9,12-tetraoxatetradecyl)carbamate

To a solution of 2-[2-[2-[2-[2-(tert-butoxycarbonylamino)ethoxy]ethoxy]ethoxy]ethoxy]ethyl 4-methylbenzenesulfonate (28 g, 56 mmol) in acetone (300 mL) was added LiBr (29.09 g, 334.9 mmol, 8.41 mL) and stirred at 60° C. for 14 hr. On completion, the reaction mixture was diluted H₂O (400 mL) and extracted with EA (150 mL×3). The combined organic layers were washed with brine (200 mL), dried over Na₂SO₄, filtered and concentrated under reduced pressure to give the title compound (21 g, 94% yield) as yellow oil. ¹H NMR (400 MHz, CHLOROFORM-d) δ=5.12-5.11 (br s, 1H), 3.81 (t, J=6.3 Hz, 2H), 3.71-3.61 (m, 12H), 3.54 (t, J=5.2 Hz, 2H), 3.47 (t, J=6.4 Hz, 2H), 3.31 (br s, 2H), 1.45 (s, 9H).

### Example 1.40. Preparation of 3-(5-(14-amino-3,6,9,12-tetraoxatetradecyl)-3-methyl-2-oxo-2,3-dihydro-1H-benzo[d]imidazol-1-yl) piperidine-2,6-dione (Intermediate AM)

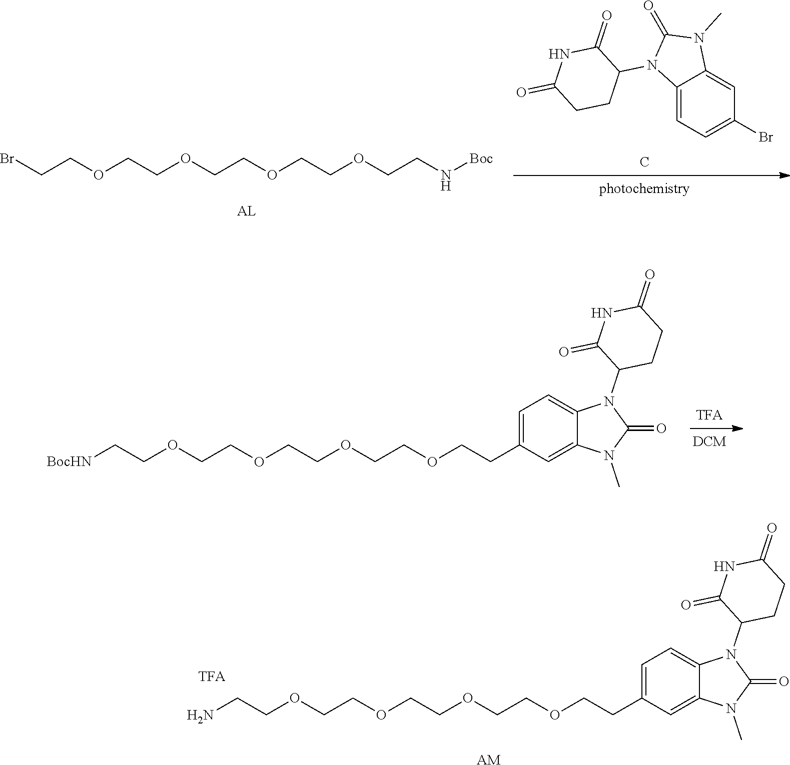

### Step 1—Tert-butyl (14-(1-(2,6-dioxopiperidin-3-yl)-3-methyl-2-oxo-2,3-dihydro-1H-benzo[d]imidazol-5-yl)-3,6,9,12-tetraoxatetradecyl)carbamate

To a solution of 3-(5-bromo-3-methyl-2-oxo-benzimidazol-1-yl)piperidine-2,6-dione (4 g, 12 mmol, Intermediate C) and tert-butyl N-[2-[2-[2-[2-(2-bromoethoxy)ethoxy]ethoxy]ethoxy]ethyl]carbamate (7.10 g, 17.74 mmol, Intermediate AL) in DME (100 mL) was added TTMSS (2.94 g, 11.8 mmol, 3.65 mL), Na₂CO₃ (2.51 g, 23.7 mmol), bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (132.71 mg, 118.29 umol) and 4-tert-butyl-2-(4-tert-butyl-2-pyridyl)pyridine; dichloronickel (235.39 mg, 591.44 umol) under N₂. The reaction was stirred and irradiated with a 34 W blue LED lamp (7 cm away), with cooling fan to keep the reaction temperature at 25° C. for 12 hr. On completion, the reaction mixture was filtered to remove the insoluble materials. The filtrate was concentrated in vacuo. The residue was purified by column chromatography (SiO₂, PE:EA=1:1 to 0:1, P: Rf=0.1) to give the crude product (6 g, 87%). The crude product was purified by reversed-phase HPLC (0.1% FA condition) to give the title compound (3.27 g, 45% yield) as white solid. H NMR (400 MHz, CHLOROFORM-d) δ=8.42 (br s, 1H), 6.98-6.88 (m, 2H), 6.72 (d, J=8.0 Hz, 1H), 5.22 (dd, J=5.3, 12.8 Hz, 1H), 5.08 (br s, 1H), 3.71-3.59 (m, 14H), 3.53 (br t, J=4.8 Hz, 2H), 3.43 (s, 3H), 3.31 (br d, J=4.8 Hz, 2H), 3.00-2.89 (m, 3H), 2.87-2.65 (m, 2H), 2.29-2.16 (m, 1H), 1.44 (s, 9H).

### Step 2—3-(5-(14-Amino-3,6,9,12-tetraoxatetradecyl)-3-methyl-2-oxo-2,3-dihydro-1H-benzo[d]imidazol-1-yl) piperidine-2,6-dione Trifluoroacetate

To a stirred mixture of tert-butyl N-{14-[1-(2,6-dioxopiperidin-3-yl)-3-methyl-2-oxo-1,3-benzodiazol-5-yl]-3,6,9,12-tetraoxatetradecan-1-yl} carbamate (150 mg, 0.259 mmol) in DCM (3 mL) was added TFA (0.6 mL) at rt. The resulting mixture was stirred for 1 h at rt. On completion, the reaction mixture was concentrated under reduced pressure. The residue was triturated with Et₂O to afford the title compound (110 mg) as a white semi-solid. LC/MS (ESI, m/z): [(M+H)]⁺=479.3.

### Example 1.41. Preparation of N-[(1R,2S)-2-fluorocyclopropyl]-8-(methylamino)-6-{[2-oxo-1-(piperidin-4-yl)pyridin-3-yl]amino}imidazo[1,2-b]pyridazine-3-carboxamide (Intermediate AN)

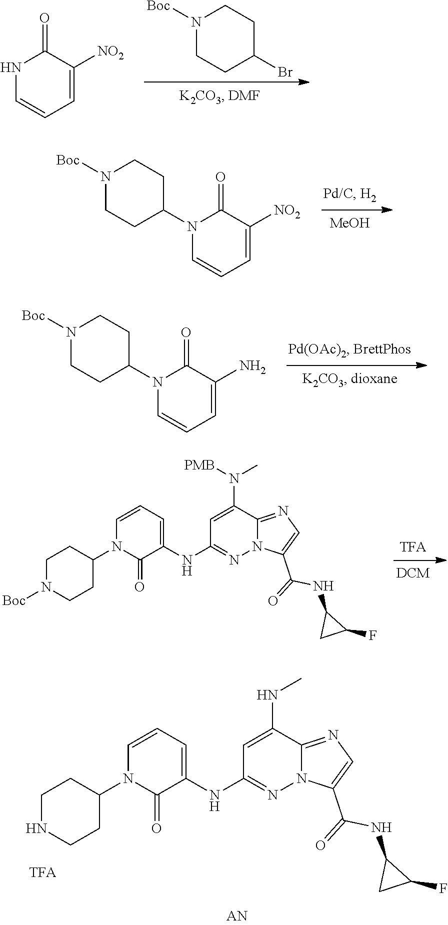

### Step 1—Tert-butyl 4-(3-nitro-2-oxopyridin-1-yl)piperidine-1-carboxylate

To a stirred solution of 3-nitro-1H-pyridin-2-one (3.54 g, 25.3 mmol) and tert-butyl 4-bromopiperidine-1-carboxylate (10.01 g, 37.90 mmol) in DMF (50 mL) was added K₂CO₃ (6.98 g, 50.5 mmol) in portions at rt under nitrogen atmosphere. The resulting mixture was stirred for 16 h at 80° C. under nitrogen atmosphere. On completion, the reaction was cooled to rt and diluted with water (200 mL), then extracted with EtOAc (3×200 mL). The combined organic layers were washed with brine (3×200 mL), and dried over anhydrous Na₂SO₄. After filtration, the filtrate was concentrated under reduced pressure. The residue was purified by reverse flash chromatography (column, C18 silica gel; mobile phase A: water (5 mmol/L NH₄HCO₃), mobile phase B: ACN, 30% to 50% gradient in 30 min; detector, UV 254 nm; the fractions were collected at 45%) and concentrated under reduced pressure to afford the title compound (6.1 g, 50% yield) as a yellow solid. LC/MS (ESI, m/z): [(M+H)]⁺=324.2.

### Step 2—Tert-butyl 4-(3-amino-2-oxopyridin-1-yl)piperidine-1-carboxylate

To a stirred solution of tert-butyl 4-(3-nitro-2-oxopyridin-1-yl)piperidine-1-carboxylate (5.9 g, 18 mmol) in THF (80 mL) was added Pd/C (0.59 g, 10 wt %) under nitrogen atmosphere. The reaction system was degassed under vacuum and purged with H₂ several times, then the reaction mixture was hydrogenated under H₂ balloon (1 atm) at 25° C. for 6 h. After completion of the reaction, Pd/C was filtered off through celite and the corresponding filtrate was concentrated under reduced pressure. The residue was purified by reverse flash chromatography (column, C18 silica gel; mobile phase A: water (5 mmol/L NH₄HCO₃), mobile phase B: ACN, 10% to 30% gradient in 25 min; detector, UV 254 nm; the fractions were collected at 25%) and concentrated under reduced pressure to afford the title compound (4.8 g, 90% yield) as a white solid. LC/MS (ESI, m/z): [(M+H)]⁺=294.2.

### Step 3—Tert-butyl 4-{3-[(3-{[(1R,2S)-2-fluorocyclopropyl]carbamoyl}-8-{[(4-methoxyphenyl)methyl](methyl)amino}imidazo[1,2-b]pyridazin-6-yl)amino]-2-oxopyridin-1-yl}piperidine-1-carboxylate

To a stirred solution of tert-butyl 4-(3-amino-2-oxopyridin-1-yl)piperidine-1-carboxylate (2 g, 7 mmol) and 6-chloro-N-[(1R,2S)-2-fluorocyclopropyl]-8-{[(4-methoxyphenyl)methyl](methyl)amino}imidazo[1,2-b]pyridazine-3-carboxamide (2.75 g, 6.82 mmol) in dioxane (40 mL) were added K₂CO₃ (1.88 g, 13.6 mmol), BrettPhos (0.37 g, 0.68 mmol) and Pd(OAc)₂ (0.15 g, 0.68 mmol) in turns at rt under nitrogen atmosphere. The resulting mixture was stirred for 4 h at 80° C. under nitrogen atmosphere. On completion, the reaction mixture was cooled to rt and filtrated. The filtrate was concentrated under reduced pressure. The residue was purified by reverse flash chromatography (column, C18 silica gel; mobile phase A: water (5 mmol/L NH₄HCO₃), mobile phase B: CAN, 10% to 50% gradient in 40 min; detector, UV 254 nm; the fractions were collected at 35%) and concentrated under reduced pressure to afford the title compound (4.3 g, 96% yield) as a black solid. LC/MS (ESI, m/z): [(M+H)]⁺=661.3.

### Step 4—N-[(1R,2S)-2-fluorocyclopropyl]-8-(methylamino)-6-{[2-oxo-1-(piperidin-4-yl)pyridin-3-yl]amino}imidazo[1,2-b]pyridazine-3-carboxamide trifluoroacetate

To a solution of tert-butyl 4-{3-[(3-{[(1R,2S)-2-fluorocyclopropyl]carbamoyl}-8-{[(4-methoxyphenyl)methyl](methyl)amino}imidazo[1,2-b]pyridazin-6-yl)amino]-2-oxopyridin-1-yl}piperidine-1-carboxylate (5.2 g, 7.9 mmol) in DCM (80 mL) was added TFA (16 mL) dropwise at rt. The reaction mixture was stirred for 6 h at rt under nitrogen atmosphere. On completion, the reaction mixture was concentrated under reduced pressure. The residue was triturated with Et₂O to afford the title compound (3.1 g) as a brown solid. LC/MS (ESI, m/z): [(M+H)]⁺=441.3.

### Example 1.42. Preparation of tert-butyl (20-bromo-3,6,9,12,15,18-hexaoxaicosyl) carbamate (Intermediate AO)

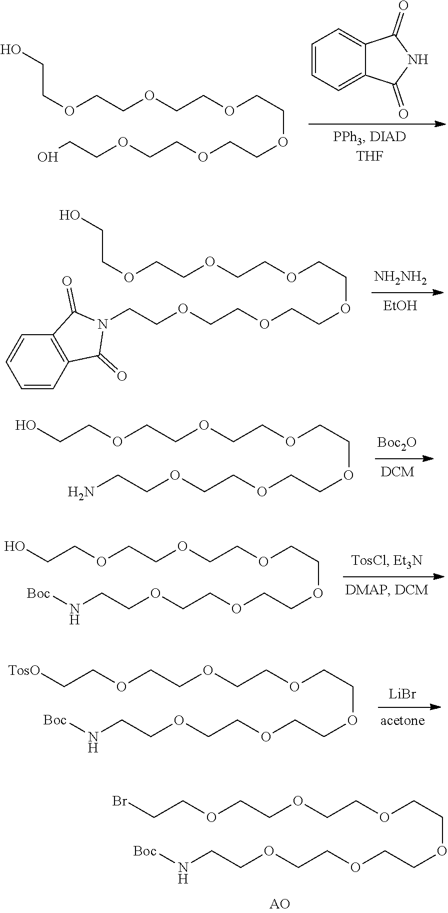

### Step 1—2-(20-Hydroxy-3,6,9,12,15,18-hexaoxaicosyl)isoindoline-1,3-dione

To a solution of 3,6,9,12,15,18-hexaoxaicosane-1,20-diol (50 g, 150 mmol, CAS #5617-32-3), isoindoline-1,3-dione (27.05 g, 183.8 mmol) and PPh₃ (60.27 g, 229.8 mmol) in THF (500 mL) was added DIAD (46.47 g, 229.8 mmol, 44.68 mL) at 0° C. The mixture was stirred at 20° C. for 2 h. On completion, the mixture was poured into H₂O (500 mL). The mixture was extracted with EA (200 mL×2). The combined organic layers were washed with brine (200 mL), dried over Na₂SO₄, filtered and concentrated under reduced pressure to give a residue. The residue was purified by column chromatography (SiO₂, PE:EA=1:1˜0:1) to afford the title compound (28 g, 61.47 mmol, 40% yield as colorless oil. ¹H NMR (400 MHz, CHLOROFORM-d) δ=7.78 (dd, J=3.2, 5.4 Hz, 2H), 7.65 (dd, J=3.2, 5.4 Hz, 2H), 3.89-3.79 (m, 2H), 3.71-3.48 (m, 26H).

### Step 2—20-Amino-3,6,9,12,15,18-hexaoxaicosan-1-ol

To a solution of 2-[2-[2-[2-[2-[2-[2-(2-hydroxyethoxy)ethoxy]ethoxy]ethoxy]ethoxy]ethoxy]ethyl]isoindoline-1,3-dione (22 g, 48 mmol) in EtOH (200 mL) was added NH₂NH₂·H₂O (7.25 g, 145 mmol, 7.04 mL) at 20° C. The mixture was stirred at 80° C. for 2 h. On completion, the mixture was filtered and filter cake was washed with EtOH (10 mL). The filtrate was concentrated in vacuo to afford the title compound (16 g) was obtained as white solid.

### Step 3—Tert-butyl (20-hydroxy-3,6,9,12,15,18-hexaoxaicosyl)carbamate

A solution of (Boc)₂O (12.34 g, 56.55 mmol, 12.99 mL) in DCM (120 mL) was added into a solution of 20-amino-3,6,9,12,15,18-hexaoxaicosan-1-ol (16 g, 49 mmol) in DCM (160 mL) at 0° C. The mixture was stirred at 15° C. for 16 h. On completion, the mixture was filtered and filter cake was washed with EA (50 mL). The filtrate was concentrated in vacuo to afford a residue. The residue was purified by column chromatography (SiO₂, PE:EA=3:1-0:1) to afford the title compound (18.5 g, 88% yield) as colorless oil. ¹H NMR (400 MHz, CHLOROFORM-d) δ=5.49-4.79 (m, 1H), 3.79-3.60 (m, 22H), 3.55 (t, J=5.1 Hz, 2H), 3.32 (t, J=5.1 Hz, 2H), 2.97-2.48 (m, 2H), 1.46 (s, 9H).

### Step 4—2,2-Dimethyl-4-oxo-3,8,11,14,17,20,23-heptaoxa-5-azapentacosan-25-yl 4-methylbenzenesulfonate

To a solution of tert-butyl N-[2-[2-[2-[2-[2-[2-(2-hydroxyethoxy) ethoxy]ethoxy]ethoxy]ethoxy] ethoxy]ethyl]carbamate (13.5 g, 31.7 mmol), Et₃N (8.03 g, 79.3 mmol, 11.04 mL) and DMAP (387.60 mg, 3.17 mmol) in DCM (130 mL) was added a solution of TosCl (9.07 g, 47.59 mmol) in DCM (90 mL) at 0° C. The mixture was stirred at 20° C. for 16 h. On completion, the mixture was concentrated in vacuo to afford a residue. The residue was purified by column chromatography (SiO₂, PE:EA=3:1-0:1) to afford the title compound (24.2 g) as colorless oil. ¹H NMR (400 MHz, CHLOROFORM-d) δ=7.81 (br d, J=8.2 Hz, 2H), 7.36 (br d, J=7.8 Hz, 2H), 5.07 (br dd, J=1.3, 3.0 Hz, 1H), 4.24-4.16 (m, 2H), 3.72-3.55 (m, 24H), 3.32 (br s, 2H), 2.46 (s, 3H), 1.46 (s, 9H).

### Step 5—Tert-butyl (20-bromo-3,6,9,12,15,18-hexaoxaicosyl)carbamate

To a solution of 2,2-dimethyl-4-oxo-3,8,11,14,17,20,23-heptaoxa-5-azapentacosan-25-yl 4-methylbenzenesulfonate (14.2 g, 24.5 mmol) in acetone (150 mL) was added LiBr (21.27 g, 244.95 mmol, 6.15 mL) at 10° C. The mixture was then stirred at 60° C. for 2 h. On completion, H₂O (200 mL) was added to the reaction and the mixture was extracted with EA (200 mL×3). The combined organic layers were washed with brine (20 mL), dried over Na₂SO₄, filtered and concentrated under reduced pressure to give the title compound (19.2 g) as colorless oil. ¹H NMR (400 MHz, CHLOROFORM-d) δ=5.27-4.86 (m, 1H), 3.81 (t, J=6.4 Hz, 2H), 3.71-3.58 (m, 20H), 3.54 (t, J=5.2 Hz, 2H), 3.48 (t, J=6.4 Hz, 2H), 3.31 (br t, J=4.8 Hz, 2H), 1.45 (s, 9H).

### Example 1.43. Preparation of 3-[5-(20-Amino-3,6,9,12,15,18-hexaoxaicosan-1-yl)-3-methyl-2-oxo-1,3-benzodiazol-1-yl]piperidine-2,6-dione (Intermediate AP)

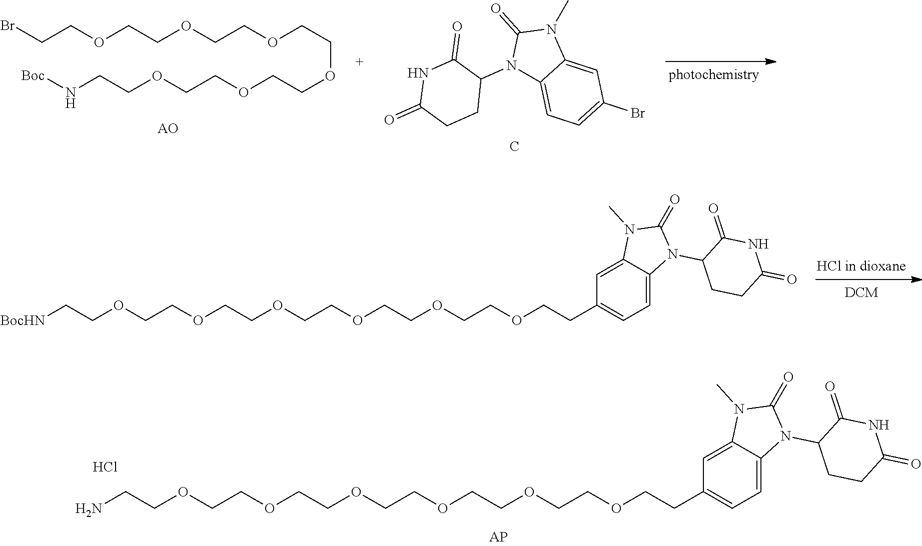

### Step 1—Tert-butyl (20-(1-(2,6-dioxopiperidin-3-yl)-3-methyl-2-oxo-2,3-dihydro-1H-benzo[d] imidazol-5-yl)-3,6,9,12,15,18-hexaoxaicosyl)carbamate

To a solution of 3-(5-bromo-3-methyl-2-oxo-2,3-dihydro-1H-benzo[d]imidazol-1-yl)piperidine-2,6-dione (3.5 g, 10.35 mmol, Intermediate C) and tert-butyl (20-bromo-3,6,9,12,15,18-hexaoxaicosyl)carbamate (5.56 g, 11.39 mmol, Intermediate AO) in DME (100 mL) was added Ir[dF(CF3)ppy]2(dtbpy)(PF6) (116.12 mg, 103.50 umol), TTMSS (2.57 g, 10.35 mmol, 3.19 mL) and Na₂CO₃ (2.19 g, 20.7 mmol) at 10° C. The reaction was then stirred at 25° C. for 16 hr irradiated with a 34 W blue LED lamp (7 cm away). On completion, the mixture was filtered and filter cake was washed with EA (100 mL). The filtrate was concentrated in vacuo to afford a residue. The residue was purified by column chromatography (SiO₂, EA:EtOH=10:1), followed by reversed-phase HPLC (0.1% FA condition) to afford the title compound (4.24 g, 6.23 mmol, 60% yield) as colorless oil. ¹H NMR (400 MHz, CHLOROFORM-d) δ=8.34 (br s, 1H), 7.03-6.88 (m, 2H), 6.74 (d, J=7.8 Hz, 1H), 5.24 (dd, J=5.4, 12.7 Hz, 1H), 5.10 (br s, 1H), 3.73-3.63 (m, 22H), 3.55 (t, J=5.2 Hz, 2H), 3.45 (s, 3H), 3.32 (br s, 2H), 3.00-2.72 (m, 5H), 2.30-2.19 (m, 1H), 1.46 (s, 9H).

### Step 2—3-[5-(20-amino-3,6,9,12,15,18-hexaoxaicosan-1-yl)-3-methyl-2-oxo-1,3-benzodiazol-1-yl]piperidine-2,6-dione Hydrochloride

To a stirred solution of tert-butyl N-{20-[1-(2,6-dioxopiperidin-3-yl)-3-methyl-2-oxo-1,3-benzodiazol-5-yl]-3,6,9,12,15,18-hexaoxaicosan-1-yl}carbamate (150 mg, 0.225 mmol) in DCM (3 mL) was added 4 M HCl (gas) in 1,4-dioxane (1 mL) at rt. The resulting mixture was stirred for 2 h at rt. On completion, the reaction mixture was concentrated under reduced pressure. The residue was triturated with Et₂O to afford the title compound (105 mg) as a white solid. LC/MS (ESI, m/z): [(M+H)]⁺=567.3.

### Example 1.44. Preparation of 3-[5-(4-aminobutyl)-3-methyl-2-oxo-1,3-benzodiazol-1-yl]piperidine-2,6-dione (Intermediate AQ)

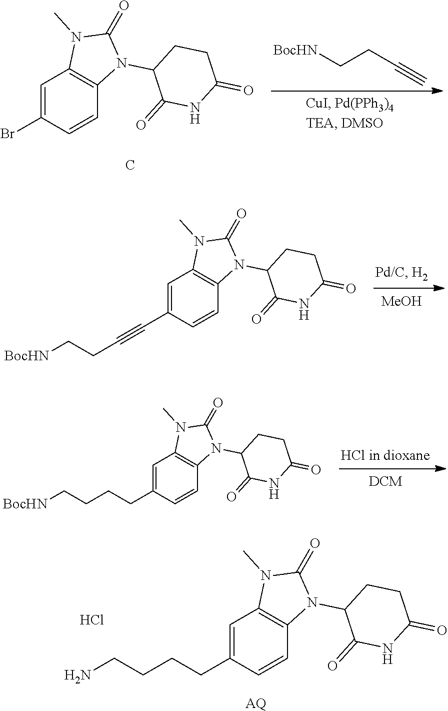

### Step 1—Tert-butyl N-[4-[1-(2,6-dioxopiperidin-3-yl)-3-methyl-2-oxo-1,3-benzodiazol-5-yl]but-3-yn-1-yl]carbamate

To a stirred mixture of 3-(5-bromo-3-methyl-2-oxo-1,3-benzodiazol-1-yl)piperidine-2,6-dione (8.00 g, 23.7 mmol, Intermediate C), tert-butyl N-(but-3-yn-1-yl)carbamate (8.01 g, 47.3 mmol) and CuI (450.55 mg, 2.366 mmol) in DMSO (40.00 mL)/TEA (20.00 mL) was added Pd(PPh₃)₄ (2.73 g, 2.36 mmol) at rt under nitrogen atmosphere. The mixture was then allowed to react for 4 h at 85° C. Upon completion, the mixture was cooled to rt and was diluted with water (100 mL). The resulting mixture was extracted with EtOAc (3×100 mL). The combined organic layers were washed with brine (3×100 mL), and dried over anhydrous Na₂SO₄. After filtration, the filtrate was concentrated under reduced pressure. The residue was purified by silica gel column chromatography, eluted with PE/EtOAc (1:2). Then the residue was further purified by reverse phase flash chromatography (Column: WelFlash™ C18-I, 20-40 ?m, 330 g; Eluent A: Water (plus 10 mmol/L FA); Eluent B: ACN; Gradient: 40%-65% B in 20 min; Flow rate: 80 mL/min; Detector: 220/254 nm; desired fractions were collected at 60% B) and concentrated under reduced pressure to afford the title compound (6 g, 60% yield) as a white solid. LC/MS (ESI, m/z): [(M+1)]⁺=427.2.

### Step 2—Tert-butyl N-[4-[1-(2,6-dioxopiperidin-3-yl)-3-methyl-2-oxo-1,3-benzodiazol-5-yl]butyl]carbamate

To a solution of tert-butyl N-[4-[1-(2,6-dioxopiperidin-3-yl)-3-methyl-2-oxo-1,3-benzodiazol-5-yl]but-3-yn-1-yl]carbamate (6.00 g, 14.1 mmol) in MeOH (800.00 mL) was added Pd/C (2.00 g, 1.88 mmol, 10 wt %) under nitrogen atmosphere. The reaction mixture was purged with hydrogen for 3 times and was stirred for 16 h at rt under hydrogen balloon. On completion, the reaction mixture was filtered through a celite pad, the filter cake was washed with MeOH (2×5 mL), and the filtrate was concentrated under reduced pressure to afford the title compound (5 g, 83% yield) as a white solid. LC/MS (ESI, m/z): [(M+1)]⁺=431.2.

### Step 3—3-[5-(4-Aminobutyl)-3-methyl-2-oxo-1,3-benzodiazol-1-yl]piperidine-2,6-dione Hydrochloride

To a solution of tert-butyl N-[4-[1-(2,6-dioxopiperidin-3-yl)-3-methyl-2-oxo-1,3-benzodiazol-5-yl]butyl]carbamate (6.00 g, 13.9 mmol) in DCM (15.00 mL) was added 4 M HCl (gas) in 1,4-dioxane (15.00 mL) dropwise under nitrogen atmosphere. The reaction mixture was stirred for 4 h at rt. On completion, the reaction mixture was concentrated under reduced pressure. The residue was triturated with Et₂O to afford the title compound (5 g, 98%) as a white solid. LC/MS (ESI, m/z): [(M+1)]⁺=331.2.

### Example 1.45. Preparation of 2-({4-[(2-Methoxyphenyl)amino]-5-(methylcarbamoyl) pyridin-2-yl}amino)pyridine-4-carboxylic Acid (Intermediate AR)

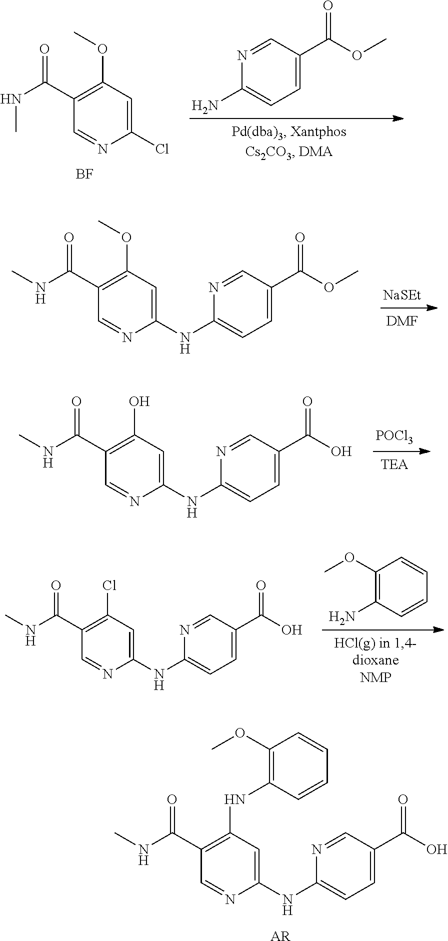

### Step 1—Methyl 6-{[4-methoxy-5-(methylcarbamoyl)pyridin-2-yl]amino}pyridine-3-carboxylate

To a stirred solution of 6-chloro-4-methoxy-N-methylpyridine-3-carboxamide (3 g, 15 mmol, Intermediate BF) and methyl 6-aminopyridine-3-carboxylate (2.73 g, 17.9 mmol) in DMA (30 mL) were added XantPhos (0.87 g, 1.5 mmol), Cs₂CO₃ (7.31 g, 22.4 mmol) and Pd₂(dba)₃ (1.37 g, 1.50 mmol) at rt under nitrogen atmosphere. The resulting mixture was stirred for additional 1 h at 110° C. under nitrogen atmosphere. On completion, the reaction mixture was cooled to rt and filtered. The filter cake was washed with DCM (3×10 mL). The filtrate was concentrated under reduced pressure. The residue was purified by reverse flash chromatography with the following conditions (column, C18 silica gel; mobile phase A: Water (0.1% FA), mobile phase B: MeCN, 5% to 35% gradient in 30 min; detector, UV 220/254 nm; desired fractions were collected at 16% B) and concentrated under reduced pressure to afford the title compound (1.8 g, 38% yield) as a white solid. ¹H NMR (400 MHz, DMSO-d₆) δ 10.43 (s, 1H), 8.85-8.77 (m, 1H), 8.58 (s, 1H), 8.16-8.11 (m, 1H), 7.99-7.88 (m, 1H), 7.79 (d, J=8.8 Hz, 1H), 7.64 (s, 1H), 3.95 (s, 3H), 3.83 (s, 3H), 2.85-2.75 (m, 3H); LC/MS (ESI, m/z): [(M+H)]⁺=317.1.

### Step 2—6-{[4-Hydroxy-5-(methylcarbamoyl) pyridin-2-yl]amino}pyridine-3-carboxylic Acid

To a stirred solution of methyl 6-{[4-methoxy-5-(methylcarbamoyl) pyridin-2-yl] amino}pyridine-3-carboxylate (500 mg, 1.6 mmol) in DMA (10 mL) was added (ethylsulfanyl) sodium (1.33 g, 15.8 mmol) at rt. The resulting mixture was stirred for 1 h at 110° C. On completion, the reaction mixture was cooled to rt and acidified to pH 6 with 4 M HCl (g) in 1,4-dioxane. The precipitated solids was collected by filtration and dried under vacuum to afford the title compound (500 mg) as a brown yellow solid. LC/MS (ESI, m/z): [(M+H)]⁺=289.1.

### Step 3—2-{[4-Chloro-5-(methylcarbamoyl)pyridin-2-yl]amino}pyridine-4-carboxylic Acid

To a stirred solution of 2-{[4-hydroxy-5-(methylcarbamoyl) pyridin-2-yl]amino}pyridine-4-carboxylic acid (430 mg, 1.49 mmol) in POCl₃ (20 mL) was added TEA (0.23 mL, 1.6 mmol) dropwise at rt. The resulting mixture was stirred for 16 h at 110° C. On completion, the reaction mixture was cooled to rt brought to pH 6 with aq. NaOH (10 M). The resulting mixture was concentrated under reduced pressure. The residue was purified by reverse flash chromatography (column, C18 silica gel; mobile phase A: water, mobile phase B: MeCN, 5% to 25% gradient in 20 min; detector, UV 220/254 nm; desired fractions were collected at 14% B) and concentrated under reduced pressure to afford the title compound (140 mg, 31% yield) as a yellow green solid. LC/MS (ESI, m/z): [(M+H)]⁺=307.0.

### Step 4—2-({4-[(2-Methoxyphenyl)amino]-5-(methylcarbamoyl)pyridin-2-yl}amino)pyridine-4-carboxylic Acid

To a stirred solution of 2-{[4-chloro-5-(methylcarbamoyl) pyridin-2-yl]amino}pyridine-4-carboxylic acid (140 mg, 0.46 mmol) and O-anisidine (281.07 mg, 2.280 mmol) in NMP (3 mL) was added a solution of 4 M HCl (gas) in 1,4-dioxane (0.36 mL, 1.4 mmol) dropwise at rt. The resulting mixture was stirred for 1 h at 120° C. On completion, the reaction mixture was cooled to rt and concentrated under reduced pressure. The residue was purified by reverse flash chromatography (column, C18 silica gel; mobile phase A: water (10 mmol/L NH₄HCO₃), mobile phase B: MeCN, 15% to 45% B gradient in 30 min; detector, UV 220/254 nm; desired fractions were collected at 18% B) and concentrated under reduced pressure to afford the title compound (50 mg, 28% yield) as a light yellow solid. LC/MS (ESI, m/z): [(M+H)]⁺=394.1.

### Example 1.46. Preparation of 3-[3-Methyl-2-oxo-4-(piperidin-3-yl)-1,3-benzodiazol-1-yl]piperidine-2,6-dione (Intermediate AS)

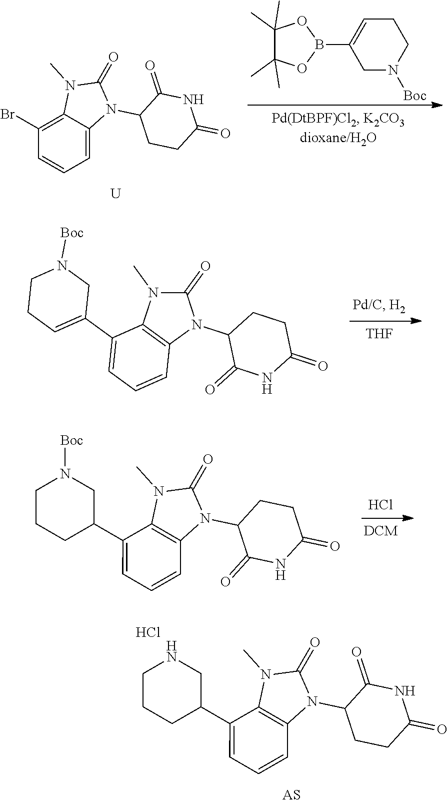

### Step 1—Tert-butyl 3-[1-(2,6-dioxopiperidin-3-yl)-3-methyl-2-oxo-1,3-benzodiazol-4-yl]-5,6-dihydro-2H-pyridine-1-carboxylate

To a stirred mixture of 3-(4-bromo-3-methyl-2-oxo-1,3-benzodiazol-1-yl) piperidine-2,6-dione (909 mg, 2.69 mmol, Intermediate U) and tert-butyl 3-(4,4,5,5-tetramethyl-1,3,2-dioxaborolan-2-yl)-5,6-dihydro-2H-pyridine-1-carboxylate (997.41 mg, 3.226 mmol, CAS #885693-20-9) in dioxane (10 mL) and water (2 mL) were added Pd(DtBPF)Cl₂ (175.19 mg, 0.269 mmol), and K₂CO₃ (743.01 mg, 5.376 mmol) at rt. The resulting mixture was stirred for 4 h at 90° C. On completion, the reaction mixture was cooled to rt and filtered. The filter cake was washed with DCM (2×10 mL). The filtrate was concentrated under reduced pressure. The residue was purified by reverse flash chromatography (column, C18 silica gel; mobile phase A: water (0.1% FA), mobile phase B: MeCN, 30% to 60% gradient in 30 min; detector, UV 220/254 nm; desired fractions were collected at 43% B) and concentrated under reduced pressure to the title compound (300 mg, 25% yield) as a white solid. ¹H NMR (400 MHz, Chloroform-d) δ 8.24 (s, 1H), 7.04 (t, J=7.8 Hz, 1H), 6.90 (d, J=7.7 Hz, 1H), 6.77 (d, J=7.8 Hz, 1H), 5.88 (s, 1H), 5.31-5.22 (m, 1H), 4.13 (s, 1H), 3.63 (s, 1H), 3.49 (s, 3H), 3.01-2.93 (m, 1H), 2.92-2.69 (m, 2H), 2.36 (s, 2H), 2.31-2.22 (m, 1H), 2.17-1.81 (m, 2H), 1.51 (s, 9H); LC/MS (ESI, m/z): [(M−H)]⁻=439.1.

### Step 2—Tert-butyl 3-[1-(2,6-dioxopiperidin-3-yl)-3-methyl-2-oxo-1,3-benzodiazol-4-yl]piperidine-1-carboxylate

To a stirred solution of tert-butyl 3-[1-(2,6-dioxopiperidin-3-yl)-3-methyl-2-oxo-1,3-benzodiazol-4-yl]-5,6-dihydro-2H-pyridine-1-carboxylate (300 mg, 0.68 mmol) in THF (10 mL) was added PtO₂ (15.47 mg, 0.068 mmol) at rt under nitrogen atmosphere. The resulting mixture was stirred for 16 h at 40° C. under hydrogen atmosphere. To above solution was added Pd/C (10 wt %, 0.1 g) under nitrogen atmosphere. The mixture was further hydrogenated at rt for 16 h under hydrogen atmosphere using a hydrogen balloon. After completion, the reaction mixture was filtered through a celite pad. The filtrate was concentrated under reduced pressure to give the title compound (180 mg) as a black oil. LC/MS (ESI, m/z): [(M−H)]⁻=441.3.

### Step 3—3-[3-Methyl-2-oxo-4-(piperidin-3-yl)-1,3-benzodiazol-1-yl]piperidine-2,6-dione hydrochloride

To a stirred solution of tert-butyl 3-[1-(2,6-dioxopiperidin-3-yl)-3-methyl-2-oxo-1,3-benzodiazol-4-yl]piperidine-1-carboxylate (80 mg, 0.18 mmol) in DCM (1.5 mL) was added 4 M HCl (gas) in 1,4-dioxane (4.11 mL) dropwise at rt. The resulting mixture was stirred for additional 1 h at rt. On completion, the reaction mixture was concentrated under reduced pressure. The residue was triturated with Et₂O to afford the title compound (60 mg) as a pink solid. LC/MS (ESI, m/z): [(M+H)]⁺=343.0.

### Example 1.47. Preparation of 3-{3-methyl-4-[methyl(pyrrolidin-3-yl)amino]-2-oxo-1,3-benzodiazol-1-yl}piperidine-2,6-dione (Intermediate AT)

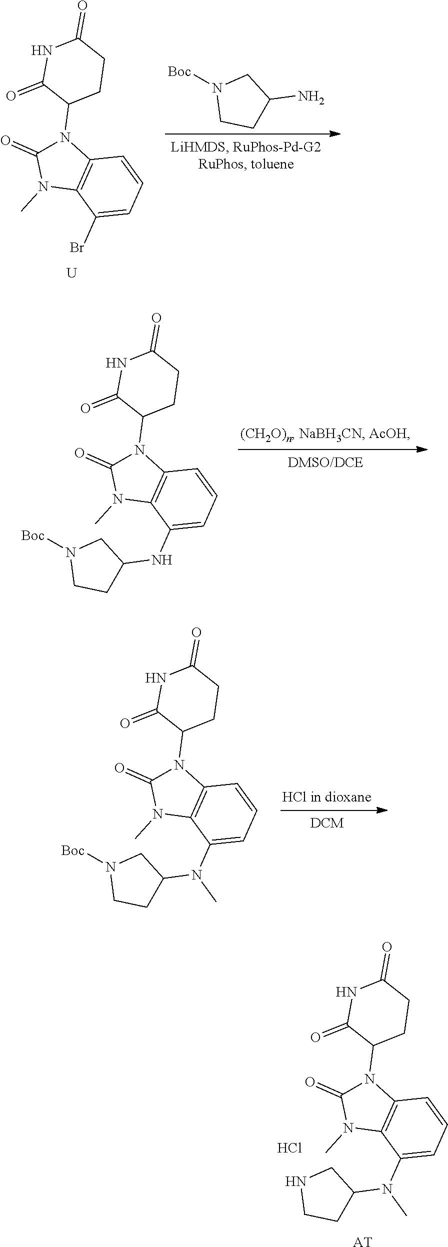

### Step 1—Tert-butyl 3-{[1-(2,6-dioxopiperidin-3-yl)-3-methyl-2-oxo-1,3-benzodiazol-4-yl]amino}pyrrolidine-1-carboxylate

To a stirred mixture of 3-(4-bromo-3-methyl-2-oxo-1,3-benzodiazol-1-yl)piperidine-2,6-dione (1 g, 3 mmol, Intermediate U) and tert-butyl 3-aminopyrrolidine-1-carboxylate (1.10 g, 5.91 mmol, CAS #186550-13-0) in methylbenzene (15 mL) were added RuPhos-PdCl-2nd G (0.46 g, 0.59 mmol) and RuPhos (0.28 g, 0.59 mmol). Next, LiHMDS (17.74 mL, 17.74 mmol) was added dropwise at rt under nitrogen atmosphere. The resulting mixture was stirred for 2 h at 80° C. under nitrogen atmosphere. On completion, the reaction mixture was cooled to rt and HCl (aq) was added until the pH was 5. The resulting mixture was filtered and the filter cake was washed with DCM (3×10 mL). The filtrate was concentrated under reduced pressure. The crude product was purified by reverse phase flash (Column: WelFlash™ C18-I, 20-40 um, 330 g; Eluent A: Water (10 mmol/L NH₄HCO₃); Eluent B: ACN; Gradient: 25%-55% B in 25 min; Flow rate: 80 mL/min; Detector: 254 nm; desired fractions were collected at 50% B) and concentrated under reduced pressure to afford the title compound (800 mg, 61% yield) as a yellow solid. LC/MS (ESI, m/z): [(M+H)]⁺=444.1.

### Step 2—Tert-butyl 3-((1-(2,6-dioxopiperidin-3-yl)-3-methyl-2-oxo-2,3-dihydro-1H-benzo[d]imidazol-4-yl)(methyl)amino)pyrrolidine-1-carboxylate

To a stirred mixture of tert-butyl 3-{[1-(2,6-dioxopiperidin-3-yl)-3-methyl-2-oxo-1,3-benzodiazol-4-yl]amino}pyrrolidine-1-carboxylate (500 mg, 1 mmol) and paraformaldehyde (507.15 mg, 5.635 mmol) in DMSO (5 mL) and DCE (5 mL) were added AcOH (0.65 mL) and KOAc (221.29 mg, 2.254 mmol). The resulting mixture was stirred for 30 min at rt. Then to above mixture was added NaBH₃CN (354.22 mg, 5.635 mmol) at rt under nitrogen atmosphere. The resulting mixture was stirred for 16 h at 60° C. under nitrogen atmosphere. On completion, the reaction mixture was concentrated under reduced pressure. The residue was purified by reverse phase flash chromatography (Column: WelFlash™ C18-I, 20-40 um, 330 g; Eluent A: Water (10 mmol/L NH₄HCO₃); Eluent B: ACN; Gradient: 25%-55% B in 25 min; Flow rate: 80 mL/min; Detector: 254 nm; desired fractions were collected at 40% B) and concentrated under reduced pressure to afford the title compound (300 mg, 58% yield) as a white solid. LC/MS (ESI, m/z): [(M+H)]⁺=458.1.

### Step 3—3-{3-Methyl-4-[methyl(pyrrolidin-3-yl)amino]-2-oxo-1,3-benzodiazol-1-yl}piperidine-2,6-dione Hydrochloride

To a stirred mixture of tert-butyl 3-((1-(2,6-dioxopiperidin-3-yl)-3-methyl-2-oxo-2,3-dihydro-1H-benzo[d]imidazol-4-yl)(methyl)amino)pyrrolidine-1-carboxylate (100 mg, 0.2 mmol) in DCM (2 mL) was added HCl (gas) in 1,4-dioxane (2 mL) dropwise at rt. The resulting mixture was stirred for 1 h at rt under nitrogen atmosphere. On completion, the reaction mixture was concentrated under reduced pressure. The residue was triturated with Et₂O to give the title compound (85 mg, 99% yield) as a yellow solid. LC/MS (ESI, m/z): [(M+H)]⁺=358.1.

### Example 1.48. Preparation of tert-butyl 2-(5-ethynylpyrimidin-2-yl)-2,6-diazaspiro[3.5]nonane-6-carboxylate (Intermediate AU)

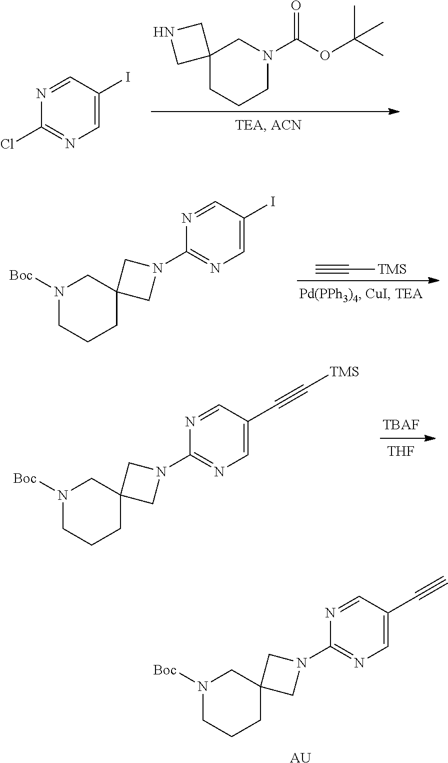

### Step 1—Tert-butyl 2-(5-iodopyrimidin-2-yl)-2,6-diazaspiro[3.5]nonane-6-carboxylate

To a solution of 2-chloro-5-iodo-pyrimidine (4.5 g, 19 mmol) and tert-butyl 2,6-diazaspiro[3.5]nonane-6-carboxylate (3.81 g, 16.8 mmol, CAS #1279844-25-5) in ACN (50 mL) was added DIEA (7.26 g, 56.2 mmol, 9.78 mL) in one portion at 25° C., then the resulting mixture was stirred at 50° C. for 12 hrs. On completion, the mixture was concentrated in vacuo. Then the residue was dissolved with H₂O (10 mL) and EtOAc (100 mL), then organic was washed with brine (10 mL×2), dried over Na₂SO₄, filtered and concentrated in vacuo. The solid was triturated with MTBE (30 mL) to afford the title compound (6.5 g, 81% yield) as a yellow solid. ¹H NMR (400 MHz, CHLOROFORM-d) δ=8.50-8.31 (m, 2H), 3.85 (br d, J=8.0 Hz, 2H), 3.73 (d, J=8.8 Hz, 2H), 3.54 (s, 2H), 3.37 (br t, J=4.4 Hz, 2H), 1.84-1.79 (m, 2H), 1.57-1.52 (m, 2H), 1.46 (s, 9H).

### Step 2—Tert-butyl 2-(5-((trimethylsilyl)ethynyl)pyrimidin-2-yl)-2,6-diazaspiro[3.5]nonane-6-carboxylate

To a solution of tert-butyl 2-(5-iodopyrimidin-2-yl)-2,8-diazaspiro[3.5]nonane-8-carboxylate (6.0 g, 14 mmol), Pd(PPh₃)₄ (1.61 g, 1.39 mmol) and CuI (265.57 mg, 1.39 mmol) in toluene (120 mL) was added TEA (4.23 g, 41.8 mmol, 5.82 mL) in one portion at 25° C. The resulting mixture was degassed with N₂ three times, then stirred at 25° C. for 0.5 hr. Next, ethynyl(trimethyl)silane (1.37 g, 13.9 mmol, 1.93 mL) was added in one portion at 25° C., then the mixture was stirred at 95° C. for 2 hrs under N₂. On completion, the reaction mixture was partitioned between H₂O (50 mL) and EtOAc (100 mL×2). The combined organic layers were dried over anhydrous sodium sulfate, filtered and concentrated under reduced pressure. The residue was purified by flash silica gel chromatography (ISCO®; 12 g SepaFlash® Silica Flash Column, Eluent of 0˜10% Ethyl acetate/Petroleum ether gradient @ 50 mL/min) to give the title compound (5.1 g, 91% yield) as a yellow solid. ¹H NMR (400 MHz, CHLOROFORM-d) δ=8.38 (s, 2H), 3.96-3.85 (m, 2H), 3.84-3.75 (m, 2H), 3.55 (s, 2H), 3.37 (t, J=5.2 Hz, 2H), 1.84-1.79 (m, 2H), 1.59-1.54 (m, 2H), 1.46 (s, 9H), 0.35-0.22 (m, 9H).

### Step 3—Tert-butyl 2-(5-ethynylpyrimidin-2-yl)-2,6-diazaspiro[3.5]nonane-6-carboxylate

To a solution of tert-butyl 2-[5-(2-trimethylsilylethynyl)pyrimidin-2-yl]-2,8-diazaspiro[3.5]nonane-8-carboxylate (5.0 g, 12 mmol) in THF (10 mL) was added TBAF (1.0 M, 62.41 mL) in one portion at 25° C., then the resulting mixture was stirred at 25° C. for 12 hrs under N₂. On completion, the mixture was poured into water (50 mL), and extracted with EtOAc (50 mL×2). The organic phase was dried over anhydrous Na₂SO₄, and concentrated in vacuo. The residue was purified by flash silica gel chromatography (ISCO®; 20 g SepaFlash® Silica Flash Column, Eluent of 30˜80% Ethylacetate/Petroleum ethergradient @ 50 mL/min) to give the title compound (4.0 g, 98% yield) as a yellow solid. ¹H NMR (400 MHz, CHLOROFORM-d) δ=8.42 (s, 2H), 3.91 (d, J=8.4 Hz, 2H), 3.80 (d, J=9.2 Hz, 2H), 3.57 (s, 2H), 3.42-3.31 (m, 2H), 3.21 (s, 1H), 1.87-1.77 (m, 2H), 1.62-1.54 (m, 2H), 1.48 (s, 9H).

### Example 1.49. Preparation of 3-{5-[2-(2-{2,6-Diazaspiro[3.5]nonan-2-yl}pyrimidin-5-yl)ethynyl]-3-methyl-2-oxo-1,3-benzodiazol-1-yl}piperidine-2,6-dione (Intermediate AV)

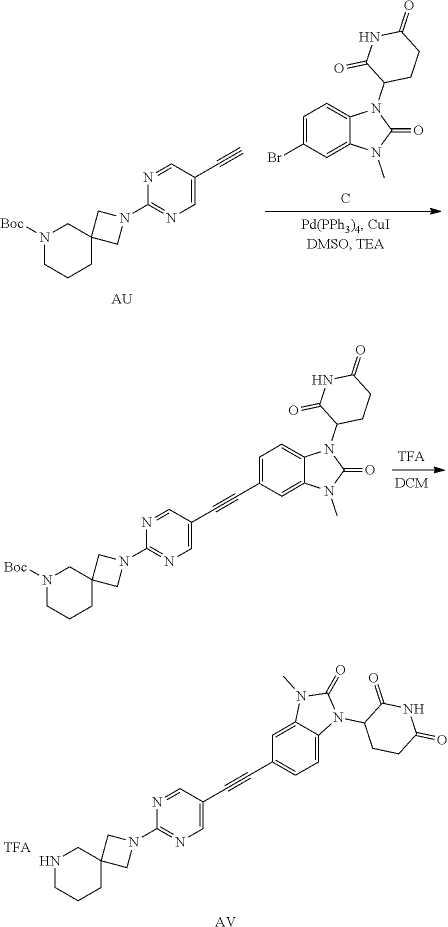

### Step 1—Tert-butyl 2-(5-((1-(2,6-dioxopiperidin-3-yl)-3-methyl-2-oxo-2,3-dihydro-1H-benzo[d] imidazol-5-yl)ethynyl)pyrimidin-2-yl)-2,6-diazaspiro[3.5]nonane-6-carboxylate

To a solution of 3-(5-bromo-3-methyl-2-oxo-2, 3-dihydro-1H-benzo[d]imidazol-1-yl)piperidine-2, 6-dione (2.25 g, 6.66 mmol, Intermediate C) in DMSO (30 mL) was added Pd(PPh₃)₄ (769.46 mg, 665.88 μmol), CuI (126.82 mg, 665.88 μmol) and TEA (2.02 g, 20 mmol, 2.78 mL). The mixture was stirred at 25° C. for 0.5 hrs. Then tert-butyl 2-(5-ethynylpyrimidin-2-yl)-2, 6-diazaspiro[3.5]nonane-6-carboxylate (2 g, 6.09 mmol, Intermediate AU) was added, and the mixture was stirred at 90° C. for 2 hrs. On completion, the reaction mixture was diluted with H₂O (150 mL) and extracted with EA (100 mL×3). The combined organic layers were washed with brine (100 mL), dried over Na₂SO₄, filtered and concentrated under reduced pressure to give a residue. The residue was purified by column chromatography (SiO₂, Petroleum ether/Ethyl acetate=100/1 to 0/1, TLC (PE/EA=0:1, Rf=0.3, uv 254 nm)) to give the title compound (2.6 g, 67% yield) as a yellow solid. ¹H NMR (400 MHz, CDCl₃-d) δ ppm 8.45 (s, 2H) 8.32 (s, 1H) 7.23-7.27 (m, 1H) 7.18 (d, J=1.2 Hz, 1H) 6.79 (d, J=8.0 Hz, 1H) 5.21 (dd, J=12.8, 5.2 Hz, 1H) 3.92 (d, J=8.4 Hz, 2H) 3.80 (d, J=9.2 Hz, 2H) 3.56 (s, 2H) 3.45 (s, 3H) 3.33-3.42 (m, 2H) 2.66-3.01 (m, 3H) 2.19-2.31 (m, 1H) 1.82-1.85 (m, 2H) 1.54-1.61 (m, 2H) 1.47 (s, 9H).

### Step 2—3-{5-[2-(2-{2,6-Diazaspiro[3.5]nonan-2-yl}pyrimidin-5-yl)ethynyl]-3-methyl-2-oxo-1,3-benzodiazol-1-yl}piperidine-2,6-dione Trifluoroacetate

To a stirred solution of tert-butyl 2-(5-{2-[1-(2,6-dioxopiperidin-3-yl)-3-methyl-2-oxo-1,3-benzodiazol-5-yl]ethynyl}pyrimidin-2-yl)-2,6-diazaspiro[3.5]nonane-6-carboxylate (60 mg, 0.1 mmol) in DCM (1.5 mL) was added TFA (0.36 mL) dropwise at rt. The resulting mixture was stirred for 1 h at rt. On completion, the reaction mixture was concentrated under reduced pressure. The residue was triturated with Et₂O to afford the title compound (45 mg) as a white solid. LC/MS (ESI, m/z): [(M+H)]⁺=486.2.

### Example 1.50. Preparation of tert-butyl 4-(3-bromocyclobutoxy)piperidine-1-carboxylate (Intermediate AW)

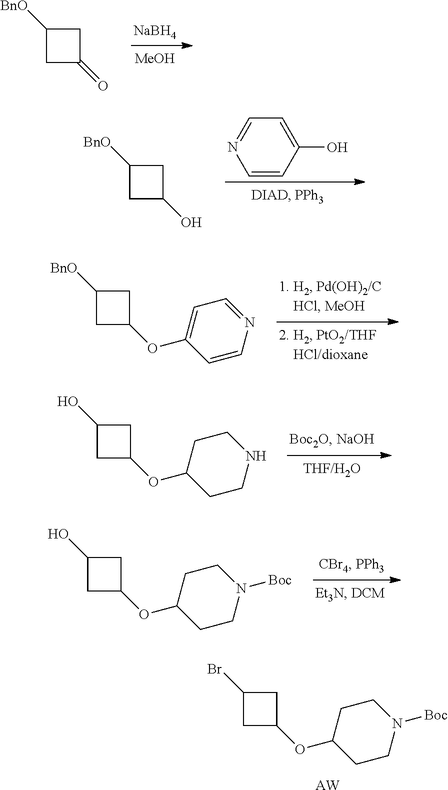

### Step 1—3-Benzyloxycyclobutanol

To a solution of 3-(benzyloxy)cyclobutanone (21 g, 119 mmol) in MeOH (150 mL) was added NaBH₄ (6.76 g, 178 mmol) in portions below 0° C., and the resulting mixture was allowed to stirred at 25° C. for 16 hours. After the reaction was completed, the reaction mixture was poured into the saturated NH₄Cl (100 mL). The mixture was concentrated in vacuo. The residue was extracted with ethyl acetate (100 mL×3). The combined organic layers were washed with brine (100 mL) and dried over Na₂SO₄. The reaction mixture was filtered and concentrated in vacuo to give the title compound (22 g, 98% yield) as yellow oil. ¹H NMR (400 MHz, DMSO-d6) δ=7.37-7.35 (m, 5H), 4.44 (s, 2H), 3.95-3.88 (q, J=7.2 Hz, 1H), 3.66-3.63 (q, J=6.8 Hz, 1H), 2.74-2.71 (m, 2H), 2.20-2.19 (m, 1H), 1.99-1.94 (m, 2H).

### Step 2—4-(3-Benzyloxycyclobutoxy)pyridine

To a solution of 3-(benzyloxy)cyclobutanol (15 g, 84 mmol), pyridin-4-ol (8.40 g, 88.3 mmol) and PPh₃ (24.2 g, 92.5 mmol) in dry toluene (150 mL) was added DIAD (20.4 g, 100 mmol, 19.6 mL) below 0° C. under N₂. Then the resulting mixture was stirred at 110° C. for 15 hr. On completion, the reaction mixture was concentrated in vacuo. The residue was triturated with ethyl acetate/petroleum ether=1:2 (300 mL), the suspension was filtrated and the filtered cake was washed with ethyl acetate/petroleum ether=1:2 (300 mL×2). The filtrate was concentrated in vacuo. The residue was purified by the reversed phase HPLC (0.5% TFA-MeCN). The solution was concentrated in vacuo and the residue was basified with solid NaHCO₃ to pH=9. The mixture was extracted with ethyl acetate (200 mL×3), the combined organic layers were washed with brine (200 mL) and dried over Na₂SO₄. After filtration and concentration, the crude product was purified by the silica gel chromatography (petroleum ether:ethyl acetate, from 100:0 to 1:4) to give the title compound (23 g, 93% yield) as white solid. ¹H NMR (400 MHz, DMSO-d6) δ=8.43-8.42 (d, J=5.2 Hz, 2H), 7.50-7.28 (m, 5H), 6.74-6.71 (m, 2H), 6.62-6.51 (m, 1H), 5.11-4.86 (m, 1H), 4.53 (s, 2H), 4.36-44.35 (m, 1H), 4.16-4.11 (m, 1H), 2.56-2.47 (m, 4H), 2.06 (d, J=1.2 Hz, 2H), 1.40-1.21 (m, 5H).

### Step 3—3-(4-Piperidyloxy)cyclobutanol

To a solution of 4-(3-(benzyloxy)cyclobutoxy)pyridine (40.5 g, 158 mmol), HCl (204 mg, 2.01 mmol, 0.2 mL, 36% solution) in MeOH (400 mL) was added a solution of Pd(OH)₂ (11.1 g, 7.93 mmol, 10 wt %) at 20° C. under N₂. The reaction mixture was stirred at 80° C. for 12 hours under H₂ (50 psi). On completion, the reaction mixture was filtered and concentrated under reduced pressure to give the title compound (28 g) as a colorless oil which was used directly in the next step. To a solution of 3-(4-pyridyloxy)cyclobutanol (5 g, 30 mmol), HCl in dioxane (4 M, 15 mL) in THF (80 mL) was added PtO₂ (1.72 g, 7.57 mmol) at 20° C. under N₂. The reaction mixture was stirred at 35° C. for 12 hours under H₂ (50 psi). On completion, the reaction mixture was filtered and concentrated under reduced pressure to give the title compound (17.4 g) as a yellow oil. ¹H NMR (400 MHz, DMSO-d6) δ=9.11-9.07 (br, 2H), 4.24-4.19 (m, 2H), 3.50 (m, 1H), 3.08 (m, 2H), 2.97-2.88 (m, 3H), 2.09-2.03 (m, 2H), 1.67 (m, 2H), 1.74-1.48 (m, 4H).

### Step 4—Tert-butyl 4-(3-hydroxycyclobutoxy)piperidine-1-carboxylate

To a solution of 3-(piperidin-4-yloxy)cyclobutanol (10 g, 58 mmol), NaOH (4.67 g, 116 mmol) in THF (130 mL) and H₂O (50 mL) was added tert-butoxycarbonyl tert-butyl carbonate (31.8 g, 146 mmol, 33.5 mL) at 25° C. under N₂. The reaction mixture was stirred at 25° C. for 12 hr. On completion, the mixture was extracted with EtOAc (100 mL×2). The combined organic layers were washed with NaCl.aq 50 mL, dried over Na₂SO₄, filtered and concentrated under reduced pressure to give a residue. The residue was purified by column chromatography (SiO₂, Petroleum ether/Ethyl acetate=1/1, the compound 5: Rf=0.4; the compound 4: Rf=0.0) to give the title compound (7 g, 4% yield) as a yellow oil. ¹H NMR (400 MHz, DMSO-d6) δ=4.54-4.51 (t, J=3.6 Hz, 1H), 4.37-1.36 (m, 1H), 3.83-3.79 (m, 2H), 3.42 (m, 1H), 3.05-2.99 (m, 2H), 2.29-2.21 (m, 4H), 1.78-1.77 (m, 2H), 1.46 (s, 11H).

### Step 5—Tert-butyl 4-(3-bromocyclobutoxy)piperidine-1-carboxylate

A solution of tert-butyl 4-(3-hydroxycyclobutoxy)piperidine-1-carboxylate (7.8 g, 29 mmol) in toluene (80 mL) at 15° C. The mixture was stirred at 110° C. for 0.5 h. To the mixture was added a solution of CBr₄ (20.9 g, 63.2 mmol) in DCM (80 mL) and Et₃N (6.40 g, 63.2 mmol, 8.80 mL) and a solution of PPh₃ (16.5 g, 63.2 mmol) in DCM (80 mL) at 15° C. The mixture was then stirred at 45° C. for 1 h, then at 15° C. for 16 h. On completion, the mixture was filtered and filter cake was washed with EtOAc (10 mL). The filtrate was concentrated in vacuo to afford a residue. The reaction mixture was diluted with H₂O (500 mL) and extracted with EtOAc (100 mL×2). The combined organic layers were dried over Na₂SO₄, filtered and concentrated under reduced pressure to give a residue. The residue was purified by column chromatography (SiO₂, Petroleum ether/Ethyl acetate=1/1, compound 6: Rf=0.85; the compound 5: Rf=0.3) to give the title compound (5.7 g, 55% yield) as a yellow oil. ¹H NMR (400 MHz, DMSO-d6) δ=3.98 (q, J=8.0 Hz, 1H), 3.89-3.87 (q, J=6.8 Hz, 1H), 3.79-3.76 (m, 2H), 3.43 (m, 1H), 3.06-2.97 (m, 4H), 2.51-2.49 (m, 2H), 1.76-1.74 (m, 2H), 1.49-1.46 (m, 11H).

### Example 1.51. Preparation of 3-{3-methyl-2-oxo-5-[3-(piperidin-4-yloxy)cyclobutyl]-1,3-benzodiazol-1-yl}piperidine-2,6-dione (Intermediate AX)

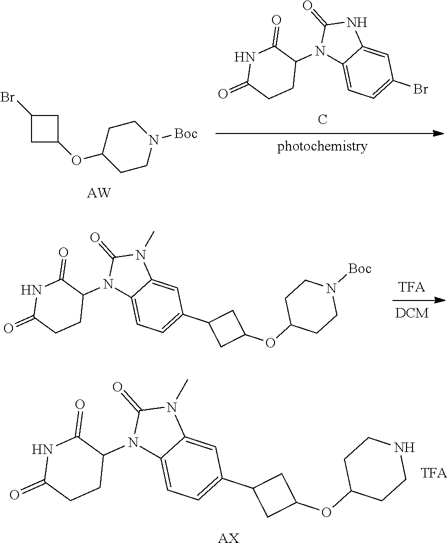

### Step 1—Tert-butyl 4-[3-[1-(2,6-dioxo-3-piperidyl)-3-methyl-2-oxo-benzimidazol-5-yl]cyclobutoxy] piperidine-1-carboxylate

To a solution of 3-(5-bromo-3-methyl-2-oxo-2,3-dihydro-1H-benzo[d]imidazol-1-yl)piperidine-2,6-dione (3.80 g, 11.2 mmol, Intermediate C) in DME (150 mL) was added Ir[dF(CF₃)ppy]₂(dtbpy)(PF₆) (126 mg, 112 umol), tert-butyl 4-(3-bromocyclobutoxy)piperidine-1-carboxylate (4.88 g, 14.6 mmol, Intermediate AW), Na₂CO₃ (2.38 g, 22.4 mmol), TTMSS (2.79 g, 11.2 mmol, 3.47 mL) and NiCl₂.dtbbpy (22.3 mg, 56.1 umol) at 25° C. The vial was sealed and placed under nitrogen atmosphere. The reaction was stirred and irradiated with a 34 W blue LED lamp (7 cm away), with cooling fan to keep the reaction temperature at 25° C. for 14 hr. On completion, the mixture was filtrated and concentrated by rotary evaporation. The residue was purified by prep-HPLC (TFA condition) to give the crude product and the crude product was recrystallized from petroleum ether (100 mL) to afford the title compound (3.61 g, 64% yield) as a white solid. ¹H NMR (400 MHz, DMSO-d6) δ=11.07 (s, 1H), 7.11-6.88 (m, 3H), 5.36-5.32 (m, 1H), 4.32-4.02 (m, 1H), 3.68-3.641 (m, 2H), 3.50 (m, 1H), 3.34 (s, 3H), 3.01-2.98 (m, 4H), 2.64-2.60 (m, 3H), 2.38 (s, 2H), 1.99-1.97 (m, 2H), 1.79-1.76 (m, 2H), 1.39 (s, 9H), 1.34-1.31 (m, 2H). LC-MS (ESI⁺) m/z 413.2 (M+H-100)⁺.

### Step 2—3-{3-methyl-2-oxo-5-[3-(piperidin-4-yloxy)cyclobutyl]-1,3-benzodiazol-1-yl}piperidine-2,6-dione Trifluoroacetate

To a stirred mixture of tert-butyl 4-{3-[1-(2,6-dioxopiperidin-3-yl)-3-methyl-2-oxo-1,3-benzodiazol-5-yl]cyclobutoxy}piperidine-1-carboxylate (75 mg, 0.15 mmol) in DCM (2 mL) was added TFA (0.4 mL) dropwise at rt. The resulting mixture was stirred for 1 h at rt. On completion, the reaction mixture was concentrated under reduced pressure. The residue was triturated with Et₂O (60 mg, 99% yield) as a white solid. LC/MS (ESI, m/z): [(M+H)]⁺=413.2.

### Example 1.52. Preparation of Tert-butyl 4-(4-oxopiperidin-1-yl)benzylcarbamate (Intermediate AY)

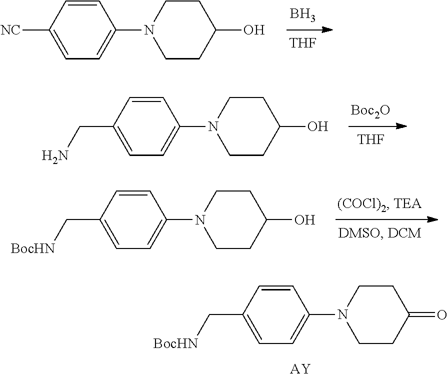

### Step 1—1-(4-(Aminomethyl)phenyl)piperidin-4-ol

To a solution of 4-(4-hydroxypiperidin-1-yl)benzonitrile (2 g, 10 mmol, CAS #79421-42-5) in THF (20 mL) was added BH₃·THF (1 M, 40 mL), then the mixture was stirred at 80° C. for 16 hr. On completion, MeOH (30 mL) was added to quench the reaction and the resulting mixture was concentrated to give the title compound (2 g) as colorless oil.

### Step 2—Tert-butyl 4-(4-hydroxypiperidin-1-yl)benzylcarbamate

To a solution of 1-[4-(aminomethyl) phenyl]piperidin-4-ol (2 g, 10 mmol) in THF (20 mL) was added Boc₂O (2.54 g, 11.6 mmol, 2.67 mL), then the mixture was stirred at 25° C. for 16 hr. On completion, the mixture was concentrated to get the residue. The residue was purified by column chromatography (SiO₂, Petroleum ether/Ethyl acetate=1/0 to 1/1) to give the title compound (2.3 g, 77% yield) as white solid. ¹H NMR (400 MHz, CHLOROFORM-d) δ=7.17 (d, J=8.4 Hz, 2H), 6.94-6.88 (m, 2H), 4.75 (br s, 1H), 4.22 (br d, J=5.5 Hz, 2H), 3.86 (tt, J=4.2, 8.8 Hz, 1H), 3.59-3.48 (m, 2H), 2.91 (ddd, J=3.1, 9.9, 12.6 Hz, 2H), 2.07-1.95 (m, 2H), 1.75-1.64 (m, 2H), 1.46 (s, 9H).

### Step 3—Tert-butyl 4-(4-oxopiperidin-1-yl)benzylcarbamate

To a mixture of (COCl)₂ (1.91 g, 15.0 mmol, 1.31 mL) in DCM (24 mL) was added a solution of DMSO (2.35 g, 30.03 mmol, 2.35 mL) in DCM (12 mL) at −78° C. and stirred for 15 min. Then the tert-butyl N-[[4-(4-hydroxy-1-piperidyl)phenyl]methyl]carbamate (2.3 g, 7.51 mmol) in DCM (12 mL) was added at −78° C. and stirred for 1 hr. Then TEA (4.56 g, 45.0 mmol, 6.27 mL) was added and the mixture was warm to 25° C. and the mixture was stirred at this temperature for 1 hr. On completion, the mixture was concentrated to get the residue. The residue was purified by column chromatography (SiO₂, Petroleum ether/Ethyl acetate=1/0 to 3/1) to give the title compound (2.28 g, 100% yield) as white solid. ¹H NMR (400 MHz, CHLOROFORM-d) δ=7.24 (d, J=8.4 Hz, 2H), 6.96 (d, J=8.4 Hz, 2H), 4.80 (br s, 1H), 4.26 (br d, J=5.6 Hz, 2H), 3.61 (t, J=6.1 Hz, 4H), 2.57 (t, J=6.0 Hz, 4H), 1.48 (s, 9H).

### Example 1.53. Preparation of 3-(3-methyl-2-oxo-5-(pyrrolidin-3-yl)-2,3-dihydro-1H-benzo[d]imidazol-1-yl)piperidine-2,6-dione (Intermediate AZ)

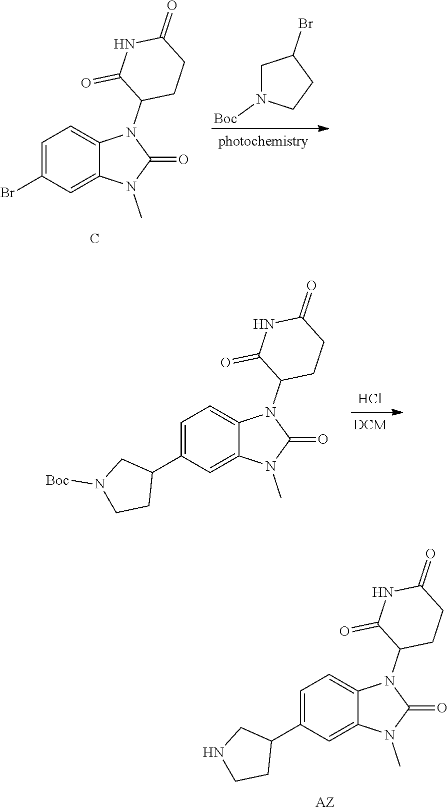

### Step 1—Tert-butyl 3-(1-(2,6-dioxopiperidin-3-yl)-3-methyl-2-oxo-2,3-dihydro-1H-benzo[d]imidazol-5-yl)pyrrolidine-1-carboxylate

To a solution of 3-(5-bromo-3-methyl-2-oxo-2, 3-dihydro-1H-benzo[d]imidazol-1-yl) piperidine-2, 6-dione (5 g, 15 mmol) and tert-butyl 3-bromopyrrolidine-1-carboxylate (3.70 g, 15 mmol, Intermediate C) in DME (180 mL) was added ditert-butyl-tetrafluoro-bis (trifluoromethyl) spiro[BLAH]; pentafluoro-λ5-phosphane; fluoride (165.89 mg, 147.86 μmol), NiCl₂.Dtbbpy (29.42 mg, 73.93 μmol), TTMSS (3.68 g, 14.8 mmol, 4.56 mL) and Na₂CO₃ (3.13 g, 29.6 mmol). The vial was sealed and placed with nitrogen gas. The reaction was stirred and irradiated with a 34 W blue LED lamp (7 cm away), with cooling fan to keep the reaction temperature at 25° C. for 14 hr. On completion, the reaction mixture was filtered and the filtrate was concentrated to get the residue. The residue was purified by reversed-phase HPLC (0.1% FA condition) to give the title compound (2.73 g, 43% yield) as yellow solid. LC-MS (ESI⁺) m/z 451.3 (M+Na)⁺.

### Step 2—3-(3-methyl-2-oxo-5-(pyrrolidin-3-yl)-2,3-dihydro-1H-benzo[d]imidazol-1-yl)piperidine-2,6-dione

To a solution of tert-butyl 3-[1-(2,6-dioxo-3-piperidyl)-3-methyl-2-oxo-benzimidazol-5-yl]pyrrolidine-1-carboxylate (1.63 g, 3.80 mmol) in DCM (15 mL) was added HCl/dioxane (4 M, 3.26 mL), then the mixture was stirred at 25° C. for 1 hr. On completion, the reaction mixture was concentrated to get the title compound (1.39 g, HCl) as yellow oil. LC-MS (ESI⁺) m/z 329.0 (M+H)⁺.

### Example 1.54. Preparation of 3-[5-(1-{1-[4-(Aminomethyl)phenyl]piperidin-4-yl}pyrrolidin-3-yl)-3-methyl-2-oxo-1,3-benzodiazol-1-yl]piperidine-2,6-dione (Intermediate BA)

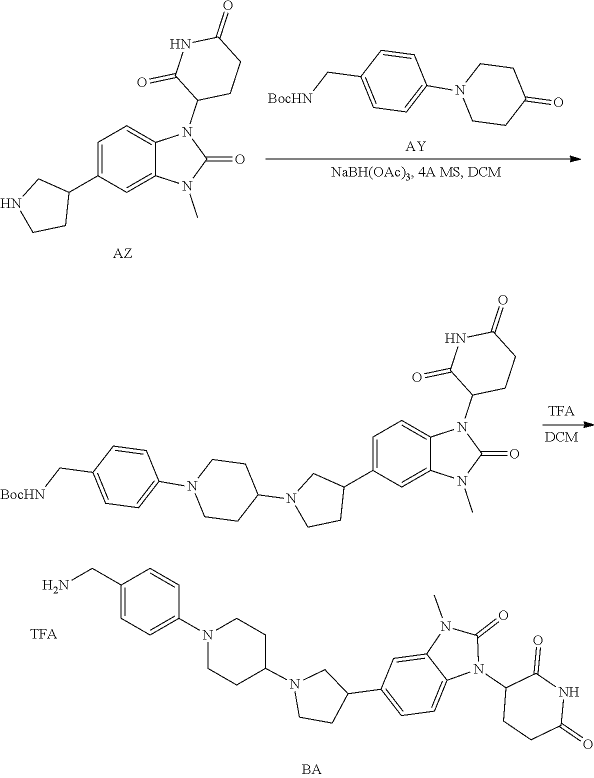

### Step 1—Tert-butyl 4-(4-(3-(1-(2,6-dioxopiperidin-3-yl)-3-methyl-2-oxo-2,3-dihydro-1H-benzo[d]imidazol-5-yl)pyrrolidin-1-yl)piperidin-1-yl)benzylcarbamate

To a solution of 3-(3-methyl-2-oxo-5-pyrrolidin-3-yl-benzimidazol-1-yl)piperidine-2,6-dione (1.39 g, 3.81 mmol, Intermediate AZ, HCl) and tert-butyl 4-(4-oxopiperidin-1-yl)benzylcarbamate (1.16 g, 3.81 mmol, Intermediate AY) in DCM (15 mL) was added NaBH (OAc)₃ (2.42 g, 11.43 mmol) and 4 Å molecular sieves (300 mg), then the mixture was stirred at 25° C. for 1 hr. On completion, the reaction mixture was concentrated to get the residue. The crude product was purified by reversed-phase HPLC (0.1% FA condition) to give the title compound (2.17 g, 85% yield, FA) as white solid. ¹H NMR (400 MHz, DMSO-d₆) δ=11.09 (s, 1H), 8.15 (s, 1H), 7.27 (s, 1H), 7.18 (s, 1H), 7.07 (t, J=8.8 Hz, 3H), 7.02-6.98 (m, 1H), 6.91 (br d, J=8.4 Hz, 2H), 5.35 (dd, J=5.2, 12.8 Hz, 1H), 4.01 (br d, J=6.0 Hz, 2H), 3.71 (br d, J=12.0 Hz, 2H), 3.56-3.38 (m, 3H), 3.33 (s, 3H), 2.99 (br s, 1H), 2.95-2.84 (m, 2H), 2.77-2.62 (m, 4H), 2.40-2.28 (m, 1H), 2.11-1.93 (m, 4H), 1.70-1.56 (m, 2H), 1.38 (s, 9H).

### Step 2—3-[5-(1-{1-[4-(Aminomethyl)phenyl]piperidin-4-yl}pyrrolidin-3-yl)-3-methyl-2-oxo-1,3-benzodiazol-1-yl]piperidine-2,6-dione Trifluoroacetate

To a stirred solution of tert-butyl N-{[4-(4-{3-[1-(2,6-dioxopiperidin-3-yl)-3-methyl-2-oxo-1,3-benzodiazol-5-yl]pyrrolidin-1-yl}piperidin-1-yl)phenyl]methyl}carbamate (150 mg, 0.243 mmol) in DCM (2 mL) was added TFA (0.8 mL) dropwise at rt. The resulting mixture was stirred for 1 h at rt under nitrogen atmosphere. On completion, the reaction mixture was concentrated under reduced pressure. The residue was triturated with diethyl ether to afford the title compound (118 mg) as a white solid. LC/MS (ESI, m/z): [(M+1)]=517.3.

### Example 1.55. Preparation of 3-{3-methyl-2-oxo-5-[2-(piperidin-4-yl)ethynyl]-1,3-benzodiazol-1-yl}piperidine-2,6-dione (Intermediate BB)

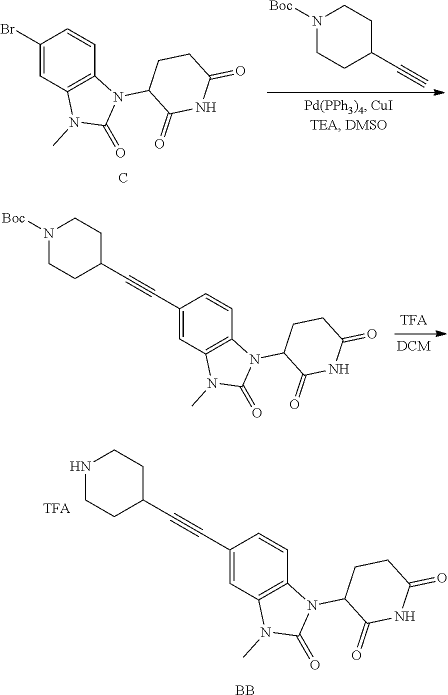

### Step 1—Tert-butyl 4-{2-[1-(2,6-dioxopiperidin-3-yl)-3-methyl-2-oxo-1,3-benzodiazol-5-yl]ethynyl}piperidine-1-carboxylate

To a stirred mixture of 3-(5-bromo-3-methyl-2-oxo-1,3-benzodiazol-1-yl)piperidine-2,6-dione (1 g, 3 mmol, Intermediate C) and tert-butyl 4-ethynylpiperidine-1-carboxylate (1.55 g, 7.39 mmol, CAS #287192-97-6) in DMSO (15 mL) were added Pd(PPh₃)₄ (0.34 g, 0.30 mmol), CuI (0.06 g, 0.3 mmol) and TEA (5 mL, 36 mmol) at rt. The resulting mixture was stirred for 2 h at 90° C. under nitrogen atmosphere. On completion, the reaction mixture was concentrated under reduced pressure. The residue was purified by reverse phase flash chromatography (Column: WelFlash™ C18-I, 20-40 um, 330 g; Eluent A: Water (plus 10 mmol/L FA); Eluent B: ACN; Gradient: 15%-55% B in 45 min; Flow rate: 80 mL/min; Detector: 254 nm; desired fractions were collected at 34% B) and concentrated under reduced pressure to afford the title compound (1.1 g, 80% yield) as a light yellow solid. LC/MS (ESI, m/z): [(M+1)]*=467.2.

### Step 2—3-{3-Methyl-2-oxo-5-[2-(piperidin-4-yl)ethynyl]-1,3-benzodiazol-1-yl}piperidine-2,6-dione Trifluoroacetate

To a stirred solution of tert-butyl 4-{2-[1-(2,6-dioxopiperidin-3-yl)-3-methyl-2-oxo-1,3-benzodiazol-5-yl]ethynyl}piperidine-1-carboxylate (1.1 g, 2.4 mmol) in DCM (15 mL) was added TFA (5 mL) dropwise at rt. The resulting mixture was stirred for 1 h at rt under nitrogen atmosphere. On completion, the reaction mixture was concentrated under reduced pressure. The residue was purified by trituration with Et₂O to give the title compound (980 mg) as a yellow solid. LC/MS (ESI, m/z): [(M+1)]⁺=367.2.

### Example 1.56. Preparation of 3-[5-(1,3-dioxolan-2-yl)pyridin-2-yl]aniline (Intermediate BC)

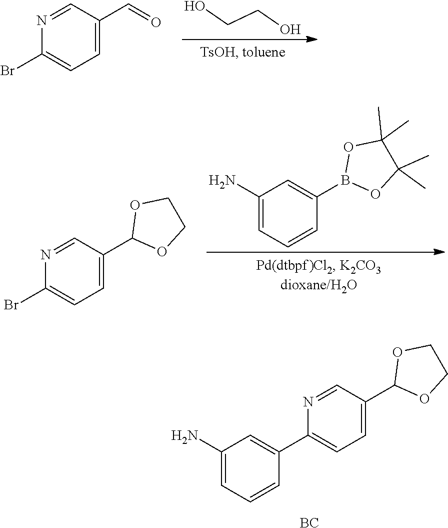

### Step 1—2-bromo-5-(1,3-dioxolan-2-yl)pyridine

To a stirred mixture of 6-bromopyridine-3-carbaldehyde (10 g, 54 mmol) and ethylene glycol (13.35 g, 215 mmol) in methylbenzene (100 mL) was added TsOH (0.93 g, 5.4 mmol) rt under nitrogen atmosphere. The resulting mixture was stirred reflux for 16 h under nitrogen atmosphere. On completion, the reaction mixture was concentrated under vacuum. The residue was diluted with EtOAc (200 mL), then washed with NaHCO₃ (3×100 mL), and dried over anhydrous Na₂SO₄. After filtration, the filtrate was concentrated under reduced pressure. The resulting mixture was concentrated under reduced pressure to give the title compound (8.1 g, 66% yield) as an off-white oil. LC/MS (ESI, m/z): [(M+H)]⁺=230.0, 232.0.

### Step 2—3-[5-(1,3-dioxolan-2-yl)pyridin-2-yl]aniline

To a stirred mixture of 2-bromo-5-(1,3-dioxolan-2-yl)pyridine (20 g, 87 mmol) and 3-(4,4,5,5-tetramethyl-1,3,2-dioxaborolan-2-yl)aniline (20.95 g, 95.63 mmol) in 1,4-dioxane (120 mL) and H₂O (40 mL) were added Pd(DtBPF)Cl₂ (5.67 g, 8.69 mmol) and K₂CO₃ (24.03 g, 173.9 mmol) at rt under nitrogen atmosphere. The resulting mixture was stirred for 2 h at 100° C. under nitrogen atmosphere. On completion, the reaction mixture was cooled to rt and concentrated under reduced pressure. The residue was purified by silica gel column chromatography, eluted with PE/EA (1:1) to afford the crude. The crude was further purified by reverse flash chromatography (column, C18 silica gel; mobile phase A: Water (10 mmol/L NH₄HCO₃), mobile phase B: MeCN, 10% to 50% gradient in 10 min; detector, UV 254 nm; desired fractions were collected at 45% B) and concentrated under reduced pressure to afford the title compound (7 g, 33% yield) as a yellow oil. ¹H NMR (400 MHz, Chloroform-d) δ 8.77 (d, J=2.2 Hz, 1H), 7.85 (dd, J=8.2, 2.2 Hz, 1H), 7.72 (dd, J=8.2, 0.9 Hz, 1H), 7.40 (t, J=2.0 Hz, 1H), 7.37-7.33 (m, 1H), 7.30-7.23 (m, 1H), 6.77-6.73 (m, 1H), 5.91 (s, 1H), 4.19-4.11 (m, 2H), 4.11-4.02 (m, 2H), 3.76 (s, 2H). LC/MS (ESI, m/z): [(M+H)]⁺=243.2.

### Example 1.57. Preparation of tert-butyl 2-[2-(2-bromoethoxy)ethoxy]acetate (Intermediate BD)

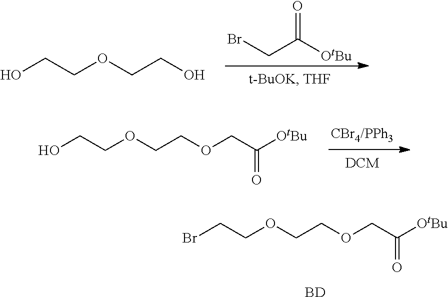 [SMILES: CC(C)(C)OC(=O)CBr ; OCCOCCO ; CC(C)(C)OC(=O)COCCOCCO ; CC(C)(C)OC(=O)COCCOCCBr ; "BD"]

### Step 1—Tert-butyl 2-[2-(2-hydroxyethoxy)ethoxy]acetate

To a solution of 2-(2-hydroxyethoxy)ethanol (10 g, 94.2 mmol, 8.93 mL) in THF (100 mL) was added t-BuOK (14.0 g, 124.7 mmol) and tert-butyl 2-bromoacetate (22.9 g, 117 mmol, 17.4 mL) at 0° C. The mixture was then stirred at 25° C. for 12 hr. On completion, the reaction mixture was quenched with EA (150 mL), filtered and filtrate was concentrated under reduced pressure to give a residue. The residue was purified by column chromatography (SiO₂, Petroleum ether/Ethyl acetate=1/1) to give the title compound (7.8 g, 13% yield) as a light yellow oil. ¹H NMR (400 MHz, CDCl₃-d) δ=4.02 (br s, 2H), 3.80-3.66 (m, 6H), 3.64 (br s, 2H), 1.51 (s, 9H).

### Step 2—Tert-butyl 2-[2-(2-bromoethoxy)ethoxy]acetate

To a solution of tert-butyl 2-[2-(2-hydroxyethoxy)ethoxy]acetate (7.8 g, 35 mmol) in DCM (300 mL) was added CBr₄ (41.1 g, 123 mmol) and PPh₃ (27.8 g, 106 mmol) at 0° C. The mixture was stirred at 25° C. for 12 hr. The reaction mixture was quenched with DCM (150 mL), filtered and the filtrate was concentrated under reduced pressure to give a residue. The residue was purified by column chromatography (SiO₂, Petroleum ether/Ethyl acetate=5/1) to give the title compound (7.1 g, 67% yield) as a colorless oil. ¹H NMR (400 MHz, CHLOROFORM-d) δ=4.04 (s, 2H), 3.84 (t, J=6.4 Hz, 2H), 3.78-3.69 (m, 4H), 3.49 (t, J=6.4 Hz, 2H), 1.49 (s, 9H).

### Example 1.58. Preparation of 2-[2-[2-[1-(2,6-dioxo-3-piperidyl)-3-methyl-2-oxo-benzimidazol-5-yl]ethoxy]ethoxy]acetic Acid (Intermediate BE)

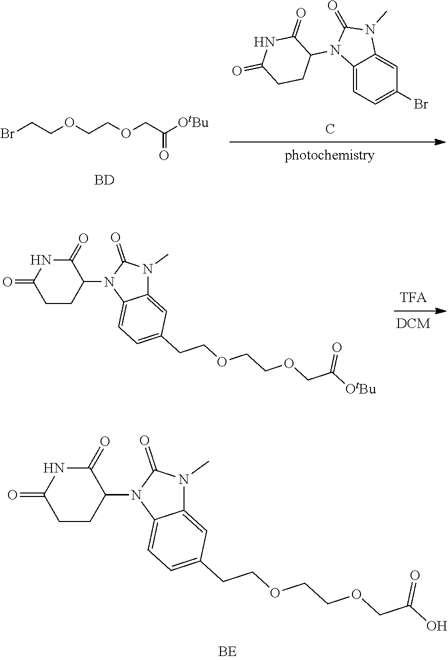

### Step 1—Tert-butyl 2-[2-[2-[1-(2,6-dioxo-3-piperidyl)-3-methyl-2-oxo-benzimidazol-5-yl]ethoxy]ethoxy]acetate

To a solution of 3-(5-bromo-3-methyl-2-oxo-benzimidazol-1-yl)piperidine-2,6-dione (4 g, 11.8 mmol, Intermediate C) in DME (130 mL) was added Ir[dF(CF₃)ppy]₂(dtbpy)(PF₆) (132 mg, 118 umol), tert-butyl 2-[2-(2-bromoethoxy)ethoxy]acetate (4.35 g, 15.3 mmol), Na₂CO₃ (2.51 g, 23.6 mmol), TTMSS (2.94 g, 11.8 mmol, 3.65 mL) and NiCl₂.dtbbpy (23.5 mg, 59.1 umol) at 25° C. The vial was sealed and placed under nitrogen atmosphere. The reaction was stirred and irradiated with a 34 W blue LED lamp (7 cm away), with cooling fan to keep the reaction temperature at 25° C. for 14 hr. On completion, the reaction mixture was filtrated and concentrated by rotary evaporation. The residue was purified by prep-HPLC (TFA condition) to give the title compound (3.20 g, 6.80 mmol, 57% yield) as light yellow solid. ¹H NMR (400 MHz, CDCl₃-d) δ=8.16 (br s, 1H), 7.00-6.89 (m, 2H), 6.73 (d, J=8.4 Hz, 1H), 5.21 (dd, J=5.2, 12.6 Hz, 1H), 4.03 (s, 2H), 3.78-3.63 (m, 6H), 3.43 (s, 3H), 3.01-2.89 (m, 3H), 2.88-2.63 (m, 2H), 2.28-2.18 (m, 1H), 1.48 (s, 9H); LC-MS (ESI⁺) m/z 406.1 (M+H-55)⁺.

### Step 2—2-[2-[2-[1-(2,6-Dioxo-3-piperidyl)-3-methyl-2-oxo-benzimidazol-5-yl]ethoxy]ethoxy]acetic Acid

To a solution of tert-butyl 2-[2-[2-[1-(2,6-dioxo-3-piperidyl)-3-methyl-2-oxo-benzimidazol-5-yl]ethoxy]ethoxy]acetate (3.20 g, 6.93 mmol) in DCM (20 mL) was added TFA (10 mL) at 0° C. The resulting mixture was stirred at 25° C. for 1 hr. On completion, the reaction mixture was concentrated under reduced pressure. The residue was purified by prep-HPLC (FA condition) to give the title compound (2.41 g, 5.82 mmol, 84% yield) as white solid. ¹H NMR (400 MHz, DMSO-d6) δ=12.57 (br s, 1H), 11.08 (s, 1H), 7.09 (s, 1H), 7.00 (d, J=8.0 Hz, 1H), 6.95-6.86 (m, 1H), 5.33 (dd, J=5.2 Hz, 1H), 4.01 (s, 2H), 3.65-3.50 (m, 6H), 3.35-3.26 (d, J=8.0 Hz, 2H), 2.95-2.80 (m, 3H), 2.77-2.57 (m, 2H), 2.04-1.95 (m, 1H); LC-MS (ESI⁺) m/z 406.1 (M+H)⁺.

### Example 1.59. Preparation of 6-Chloro-4-methoxy-N-methylpyridine-3-carboxamide (Intermediate BF)

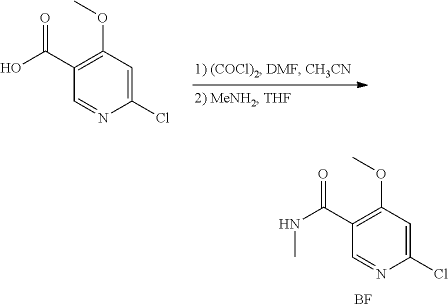

To a stirred solution of 6-chloro-4-methoxypyridine-3-carboxylic acid (30 g, 160 mmol, CAS #716362-10-6) in ACN (500 mL) was treated with (COCl)₂ (0.34 mL, 4 mmol) for 1 min at rt under nitrogen atmosphere followed by the addition of DMF (1.24 mL, 16 mmol) dropwise at rt. The reaction was stirred for 30 min at rt. Once acyl chloride was generated, the resulting mixture was concentrated under reduced pressure. The residue was re-dissolved in CH₃CN (200 mL) and added dropwise it to 1 M CH₃NH₂ (6.65 mL) THF solution. On completion, the reaction mixture was concentrated under reduced pressure. The residue was triturated with n-hexane to afford the title compound (22 g, 69% yield) as a brown solid. LC/MS (ESI, m/z): [(M+H)]⁺=201.1.

### Example 1.60. Preparation of tert-butyl 14-bromo-3,6,9,12-tetraoxatetradecan-1-oate (Intermediate BG)

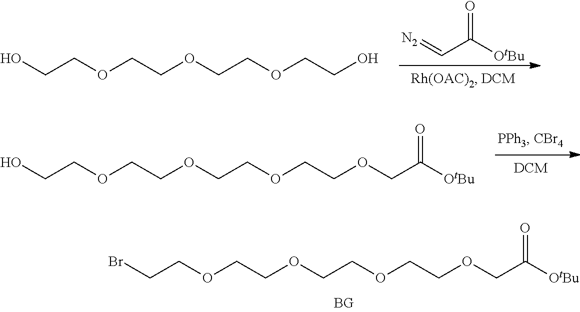

### Step 1—Tert-butyl 14-hydroxy-3,6,9,12-tetraoxatetradecan-1-oate

To a solution of 2-[2-[2-(2-hydroxyethoxy)ethoxy]ethoxy]ethanol (50 g, 250 mmol, 44.25 mL) and Rh(OAc)₂ (568.90 mg, 2.57 mmol) in DCM (350 mL) was added tert-butyl 2-diazoacetate (47.57 g, 334.6 mmol) in DCM (150 mL) at 0° C. The mixture was stirred at 20° C. for 16 hr. On completion, the reaction mixture was quenched with H₂O (200 mL) and extracted with EA (150 mL×3). The combined organic layers were washed with brine (200 mL), dried over Na₂SO₄, filtered and concentrated under reduced pressure to give a residue. The residue was purified by column chromatography (SiO₂, PE:EA=3:1 to 0:1, P: Rf=0.1) to give the title compound (11 g, 35.67 mmol, 14% yield) as yellow oil. ¹H NMR (400 MHz, CHLOROFORM-d) δ=4.04 (s, 2H), 3.77-3.66 (m, 14H), 3.65-3.60 (m, 2H), 1.49 (s, 9H).

### Step 2—Tert-butyl 14-bromo-3,6,9,12-tetraoxatetradecan-1-oate

To a solution of tert-butyl 2-[2-[2-[2-(2-hydroxyethoxy)ethoxy]ethoxy]ethoxy]acetate (11 g, 36 mmol) and PPh₃ (28.07 g, 107.0 mmol) in DCM (120 mL) was added CBr₄ (35.49 g, 107 mmol) at 0° C. The mixture was stirred at 25° C. for 3 hr. On completion, the reaction mixture was filtered to remove the insoluble materials. The filtrate was concentrated in vacuo. The residue was purified by column chromatography (SiO₂, PE:EA=3:1 to 0:1, P: Rf=0.4) to give the title compound (9 g, 68% yield) as yellow oil. ¹H NMR (400 MHz, CHLOROFORM-d) δ=3.95 (s, 2H), 3.76-3.72 (m, 2H), 3.66-3.59 (m, 12H), 3.43-3.38 (m, 2H), 1.41 (s, 9H).

### Example 1.61. Preparation of 14-(1-(2,6-dioxopiperidin-3-yl)-3-methyl-2-oxo-2,3-dihydro-1H-benzo[d]imidazol-4-yl)-3,6,9,12-tetraoxatetradecan-1-oic Acid (Intermediate BH)

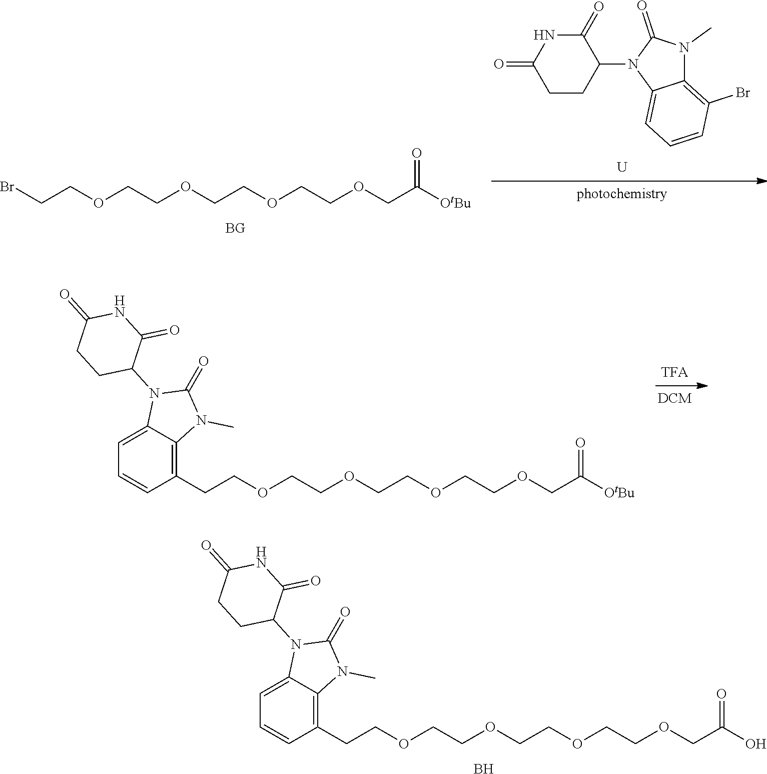

### Step 1—Tert-butyl 14-(1-(2,6-dioxopiperidin-3-yl)-3-methyl-2-oxo-2,3-dihydro-1H-benzo[d]imidazol-4-yl)-3,6,9,12-tetraoxatetradecan-1-oate

To a solution of tert-butyl 2-[2-[2-[2-(2-bromoethoxy)ethoxy]ethoxy]ethoxy]acetate (4.83 g, 13.01 mmol, Intermediate BG) and 3-(4-bromo-3-methyl-2-oxo-benzimidazol-1-yl)piperidine-2,6-dione (4 g, 12 mmol, Intermediate U) in DME (60 mL) was added TTMSS (2.94 g, 11.8 mmol, 3.65 mL), Na₂CO₃ (2.51 g, 23.7 mmol), Ir[dF(CF3)ppy]2(dtbpy)(PF6) (132.71 mg, 118.29 umol) and NiCl2.dtbbpy (23.54 mg, 59.14 umol) under N₂. The reaction was stirred and irradiated with a 34 W blue LED lamp (7 cm away), with cooling fan to keep the reaction temperature at 25° C. for 14 hr. On completion, the reaction mixture was filtered to remove the insoluble materials. The filtrate was concentrated in vacuo. The residue was purified by column chromatography (SiO₂, PE:EA=3:1 to 0:1, P: Rf=0.3), followed by reversed-phase HPLC (0.1% FA condition) to give the title compound (3 g) as colorless oil. ¹H NMR (400 MHz, CHLOROFORM-d) δ=8.17 (s, 1H), 6.96-6.89 (m, 1H), 6.88-6.83 (m, 1H), 6.61 (dd, J=1.0, 8.0 Hz, 1H), 5.20-5.08 (m, 1H), 3.94 (s, 2H), 3.70-3.59 (m, 10H), 3.57-3.54 (m, 7H), 3.15 (t, J=7.2 Hz, 2H), 2.93-2.59 (m, 3H), 2.19-2.09 (m, 1H), 1.40 (s, 9H).

### Step 2—14-(1-(2,6-Dioxopiperidin-3-yl)-3-methyl-2-oxo-2,3-dihydro-1H-benzo[d]imidazol-4-yl)-3,6,9,12-tetraoxatetradecan-1-oic Acid

To a solution of tert-butyl 2-[2-[2-[2-[2-[1-(2,6-dioxo-3-piperidyl)-3-methyl-2-oxo-benzimidazol-4-yl]ethoxy]ethoxy]ethoxy]ethoxy]acetate (2.9 g, 5.3 mmol) in DCM (30 mL) was added TFA (23.10 g, 202.6 mmol, 15 mL). The mixture was stirred at 20° C. for 2 hr. On completion, the reaction mixture was concentrated in vacuo. The crude product was purified by reversed-phase HPLC (0.1% FA condition) to give the title compound (2.18 g, 83% yield) as white solid. ¹H NMR (400 MHz, CHLOROFORM-d) δ=8.59 (s, 1H), 7.05-6.98 (m, 1H), 6.97-6.91 (m, 1H), 6.71 (d, J=7.8 Hz, 1H), 5.27 (br dd, J=5.3, 12.4 Hz, 1H), 4.15 (s, 2H), 3.80-3.71 (m, 7H), 3.70-3.59 (m, 10H), 3.23 (t, J=6.8 Hz, 2H), 2.99-2.68 (m, 3H), 2.28-2.18 (m, 1H).

### Example 1.62. Preparation of 14-(1-(2,6-Dioxopiperidin-3-yl)-3-methyl-2-oxo-2,3-dihydro-1H-benzo[d]imidazol-5-yl)-3,6,9,12-tetraoxatetradecan-1-oic Acid (Intermediate BI)

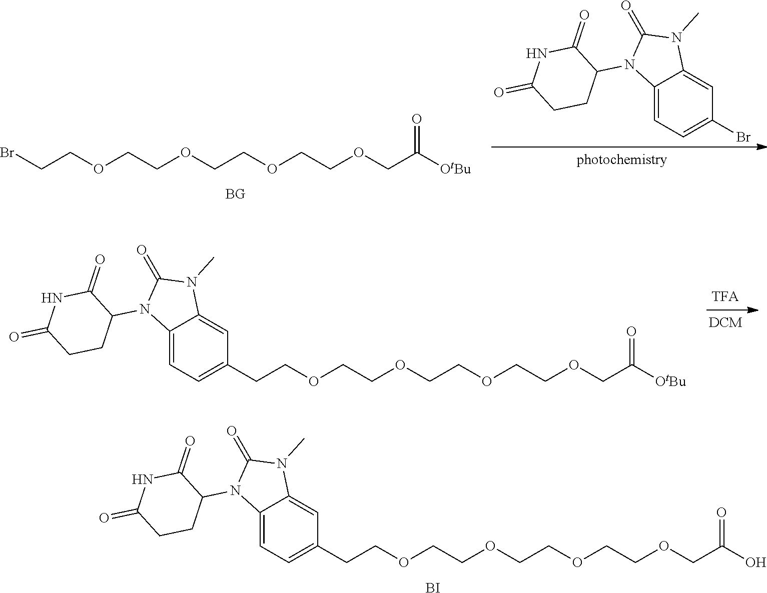

### Step 1—Tert-butyl (14-hydroxy-3,6,9,12-tetraoxatetradecyl)carbamatetert-butyl 14-(1-(2,6-dioxopiperidin-3-yl)-3-methyl-2-oxo-2,3-dihydro-1H-benzo[d]imidazol-5-yl)-3,6,9,12-tetraoxatetradecan-1-oate

To a solution of tert-butyl 2-[2-[2-[2-(2-bromoethoxy)ethoxy]ethoxy]ethoxy]acetate (4.83 g, 13 mmol, Intermediate BG) and 3-(5-bromo-3-methyl-2-oxo-benzimidazol-1-yl)piperidine-2,6-dione (4 g, 12 mmol, Intermediate C) in DME (50 mL) was added TTMSS (2.94 g, 11.83 mmol, 3.65 mL), Na₂CO₃ (2.51 g, 23.7 mmol), Ir[dF(CF3)ppy]2(dtbpy)(PF6) (132.71 mg, 118.29 umol) and NiCl2.dtbbpy (23.54 mg, 59.14 umol) under N₂. The reaction was stirred and irradiated with a 34 W blue LED lamp (7 cm away), with cooling fan to keep the reaction temperature at 25° C. for 14 hr. On completion, the reaction mixture was filtered to remove the insoluble materials. The filtrate was concentrated in vacuo. The residue was purified by column chromatography (SiO₂, PE:EA=3:1 to 0:1, P: Rf=0.3), followed by reversed-phase HPLC (0.1% FA condition) to give the title compound (3.5 g, 54% yield) as yellow oil. ¹H NMR (400 MHz, CHLOROFORM-d) δ=8.34 (br s, 1H), 6.98-6.88 (m, 2H), 6.73 (d, J=8.0 Hz, 1H), 5.22 (dd, J=5.3, 12.8 Hz, 1H), 4.02 (s, 2H), 3.73-3.60 (m, 14H), 3.43 (s, 3H), 2.97-2.64 (m, 5H), 2.28-2.16 (m, 1H), 1.47 (s, 9H).

### Step 2—14-(1-(2,6-Dioxopiperidin-3-yl)-3-methyl-2-oxo-2,3-dihydro-1H-benzo[d]imidazol-5-yl)-3,6,9,12-tetraoxatetradecan-1-oic Acid

To a solution of tert-butyl 2-[2-[2-[2-[2-[1-(2,6-dioxo-3-piperidyl)-3-methyl-2-oxo-benzimidazol-5-yl]ethoxy]ethoxy]ethoxy]ethoxy]acetate (3.4 g, 6.2 mmol) in DCM (34 mL) was added TFA (26.18 g, 229.6 mmol, 17.00 mL). The mixture was stirred at 20° C. for 4 hr. The mixture reaction was concentrated in vacuo. The crude product was purified by reversed-phase HPLC (0.1% FA condition) to give the title compound (2.8 g, 91% yield) as white solid. ¹H NMR (400 MHz, CHLOROFORM-d) δ=8.61 (s, 1H), 7.00-6.86 (m, 2H), 6.75 (d, J=8.0 Hz, 1H), 5.24 (dd, J=5.3, 12.8 Hz, 1H), 4.14 (s, 2H), 3.75-3.62 (m, 14H), 3.42 (s, 3H), 2.98-2.64 (m, 5H), 2.27-2.14 (m, 1H).

### Example 1.63. Preparation of 3-(5-(4-(6-aminopyridin-3-yl)piperazin-1-yl)-3-methyl-2-oxo-2,3-dihydro-1H-benzo[d]imidazol-1-yl)piperidine-2,6-dione (Intermediate BJ)

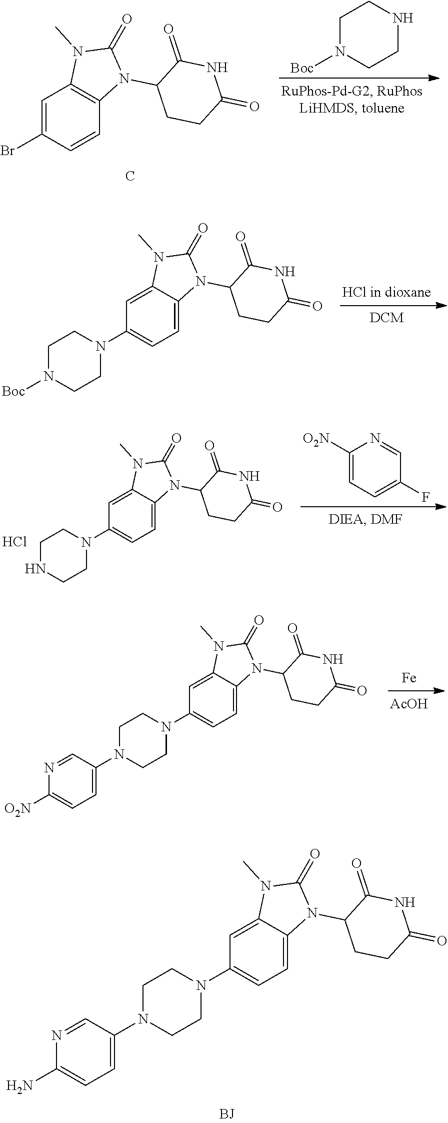

### Step 1—Tert-butyl 4-(1-(2,6-dioxopiperidin-3-yl)-3-methyl-2-oxo-2,3-dihydro-1H-benzo[d]imidazol-5-yl)piperazine-1-carboxylate

To a stirred solution of 3-(5-bromo-3-methyl-2-oxo-1,3-benzodiazol-1-yl)piperidine-2,6-dione (2.00 g, 5.91 mmol, Intermediate C) and tert-butyl piperazine-1-carboxylate (1.65 g, 8.87 mmol) in toluene (30 mL) were added RuPhos-PdCl-2nd G (0.92 g, 1.18 mmol), RuPhos (0.55 g, 1.18 mmol), followed by addition 1 M LiHMDS THF solution (35.484 mL 35.484 mmol) in nitrogen atmosphere. The resulting mixture was stirred under nitrogen atmosphere at 80° C. for 2 h. On completion, the mixture was cooled to rt and acidified to pH 2 with FA. The resulting mixture was concentrated under reduced pressure. The residue was dissolved in DMSO (20 mL). The residue was purified by reverse phase flash chromatography (Column: WelFlash™ C18-I, 20-40 um, 330 g; Eluent A: Water (plus 10 mmol/L FA); Eluent B: ACN; Gradient: 20%-50% B in 25 min; Flow rate: 80 mL/min; Detector: 254 nm; desired fractions were collected at 37% B) and concentrated under reduced pressure to afford the title compound (0.84 g, 32% yield) as a brown solid. LC/MS (ESI, m/z): [(M+H)]⁺=444.2.

### Step 2—3-(3-Methyl-2-oxo-5-(piperazin-1-yl)-2,3-dihydro-1H-benzo[d]imidazol-1-yl)piperidine-2,6-dione Hydrochloride

To a stirred solution of tert-butyl 4-(1-(2,6-dioxopiperidin-3-yl)-3-methyl-2-oxo-2,3-dihydro-1H-benzo[d]imidazol-5-yl)piperazine-1-carboxylate (0.84 g, 1.9 mmol) in DCM (20 mL) was added a solution of 4 M HCl (gas) in 1,4-dioxane (10 mL) dropwise at rt. The resulting mixture was stirred for additional 1 h at rt. On completion, the reaction mixture was concentrated under reduced pressure. The residue was triturated with Et₂O (20 mL) to afford the title compound (0.69 g) as a brown solid. LC/MS (ESI, m/z): [(M+H)]⁺=344.0.

### Step 3—3-(3-Methyl-5-(4-(6-nitropyridin-3-yl)piperazin-1-yl)-2-oxo-2,3-dihydro-1H-benzo[d]imidazol-1-yl)piperidine-2,6-dione

To a stirred solution of 3-(3-methyl-2-oxo-5-(piperazin-1-yl)-2,3-dihydro-1H-benzo[d]imidazol-1-yl)piperidine-2,6-dione hydrochloride (1.00 g, 2.63 mmol) and 5-fluoro-2-nitropyridine (0.45 g, 3.2 mmol) in DMF (10 mL) was added DIEA (1.38 ml, 7.90 mmol) at rt under nitrogen atmosphere. The resulting mixture was stirred for 2 h at 80° C. under nitrogen atmosphere. On completion, the residue was purified by reverse phase flash chromatography (Column: WelFlash™ C18-I, 20-40 um, 330 g; Eluent A: Water (plus 10 mmol/L FA); Eluent B: ACN; Gradient: 25%-55% B in 25 min; Flow rate: 80 mL/min; Detector: 254 nm; desired fractions were collected at 42% B) and concentrated under reduced pressure to afford the title compound (0.64 g, 52% yield) as a brown solid. LC/MS (ESI, m/z): [(M+H)]⁺=466.1.

### Step 4—3-(5-(4-(6-Aminopyridin-3-yl)piperazin-1-yl)-3-methyl-2-oxo-2,3-dihydro-1H-benzo[d]imidazol-1-yl)piperidine-2,6-dione

To a solution of 3-(3-methyl-5-(4-(6-nitropyridin-3-yl)piperazin-1-yl)-2-oxo-2,3-dihydro-1H-benzo[d]imidazol-1-yl)piperidine-2,6-dione (640.00 mg, 1.375 mmol) in AcOH (10 mL) was added iron powder (383.92 mg, 6.875 mmol) under nitrogen atmosphere. The resulting mixture was stirred for 16 h at rt under nitrogen atmosphere. On completion, the reaction mixture was concentrated under reduced pressure. The residue was dissolved in DMSO (10 mL) and purified by reverse phase flash chromatography (Column: WelFlash™ C18-I, 20-40 um, 330 g; Eluent A: Water (plus 10 mmol/L FA); Eluent B: ACN; Gradient: 5%-35% B in 25 min; Flow rate: 80 mL/min; Detector: 220/254 nm; desired fractions were collected at 10% B) and concentrated under reduced pressure to afford the title compound (465 mg, 78% yield) as a brown solid. LC/MS (ESI, m/z): [(M+H)]⁺=436.1.

### Example 1.64. Preparation of tert-butyl 4-(prop-2-yn-1-yloxy)piperidine-1-carboxylate (Intermediate BK)

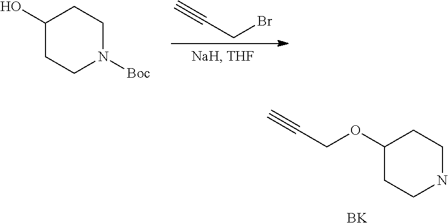

To a solution of tert-butyl 4-hydroxypiperidine-1-carboxylate (25.0 g, 124 mmol) in THF (300 mL) was added NaH (5.96 g, 149 mmol, 60% dispersion in mineral oil) at 0° C. and the suspension was stirred at 0° C. for 0.5 hr. Then 3-bromoprop-1-yne (22.2 g, 149 mmol, 16.1 mL, 80% solution) was added into the mixture at 0° C. and the resulting mixture was stirred at 25° C. for 12 hr. On completion, the reaction was quenched by addition of the saturated NH₄Cl aqueous (200 mL), then extracted with EA (2000 mL×3). The organic layer was washed with brine (100 mL), dried over Na₂SO₄, and the solvent was removed under reduced pressure to give a residue. The residue was purified by column chromatography (SiO₂, PE:EA=8:1 to 5:1, P: Rf=0.5) to give the title compound (26 g, 88% yield) as yellow solid. ¹H NMR (400 MHz, CHLOROFORM-d) δ=4.13 (d, J=2.4 Hz, 2H), 3.72-3.63 (m, 3H), 3.07-3.00 (m, 2H), 2.35-2.34 (t, J=2.4 Hz, 1H), 1.79-1.76 (m, 2H), 1.49-1.44 (m, 2H), 1.39 (s, 9H).

### Example 1.65. Preparation of 3-{3-Methyl-2-oxo-5-[3-(piperidin-4-yloxy)prop-1-yn-1-yl]-1,3-benzodiazol-1-yl}piperidine-2,6-dione (Intermediate BL)

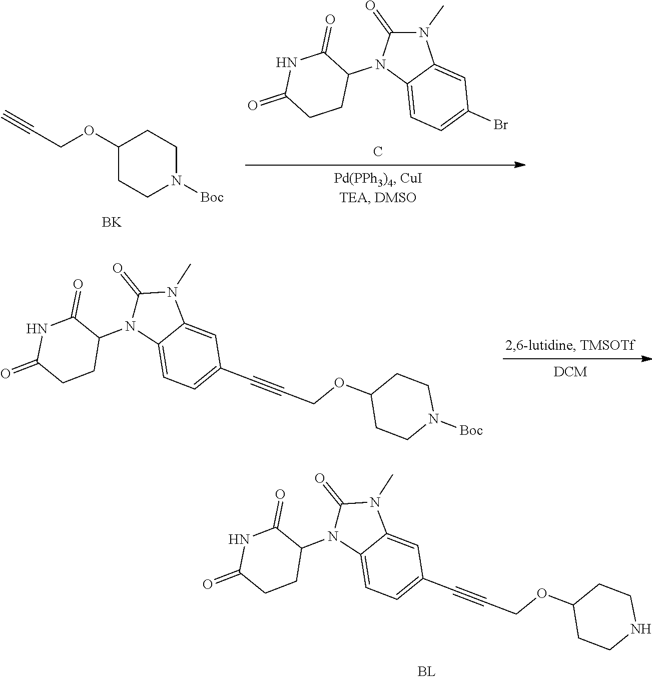

### Step 1—tert-butyl 4-((3-(1-(2,6-dioxopiperidin-3-yl)-3-methyl-2-oxo-2,3-dihydro-1H-benzo[d]imidazol-5-yl)prop-2-yn-1-yl)oxy)piperidine-1-carboxylate

A mixture of tert-butyl 4-prop-2-ynoxypiperidine-1-carboxylate (7.43 g, 31.1 mmol, Intermediate BK), 3-(5-bromo-3-methyl-2-oxo-benzimidazol-1-yl) piperidine-2,6-dione (7 g, 20 mmol, Intermediate C), CuI (394.24 mg, 2.07 mmol), Pd(PPh₃)₄ (2.39 g, 2.07 mmol) and TEA (20.95 g, 207.0 mmol, 28.81 mL) in DMSO (120 mL) was degassed and purged with N₂ for 3 times in a glove box. The mixture was stirred at 80° C. for 12 hr under N₂ atmosphere. On completion, H₂O (300 mL) was added into the mixture, and the solution was extracted with EtOAc (200 mL×3). The combined organic layer was washed with brine (300 mL) and dried over Na₂SO₄, the solvent was removed under reduced pressure to give a residue. The residue was purified by column chromatography (SiO₂, PE:EA=3:1 to 0:1, Rf=0.6) to give the crude product (8 g, 86% purity). The product was then purified by reversed-phase HPLC (0.1% FA condition) to give the title compound (4.82 g, 45% yield) as off-white solid. ¹H NMR (400 MHz, CHLOROFORM-d) δ=8.12 (s, 1H), 7.21-7.19 (d, J=8.4 Hz, 1H), 7.12 (s, 1H), 6.76-6.74 (d, J=8 Hz, 1H), 5.23-5.18 (dd, J=5.2 Hz, 12.8 Hz, 1H), 4.43 (s, 2H), 3.82-3.76 (m, 3H), 3.43 (s, 3H), 3.16-3.10 (m, 2H), 2.94-3.75 (m, 3H), 2.27 (m, 1H), 1.91-1.89 (m, 2H), 1.62-1.57 (m, 2H), 1.47 (s, 9H).

### Step 2—3-{3-methyl-2-oxo-5-[3-(piperidin-4-yloxy)prop-1-yn-1-yl]-1,3-benzodiazol-1-yl}piperidine-2,6-dione

To a stirred solution of tert-butyl 4-({3-[1-(2,6-dioxopiperidin-3-yl)-3-methyl-2-oxo-1,3-benzodiazol-5-yl]prop-2-yn-1-yl}oxy)piperidine-1-carboxylate (200 mg, 0.4 mmol) in DCM (3 mL) were added 2,6-lutidine (863.18 mg, 8.06 mmol) and TMSOTf (895.15 mg, 4.03 mmol) dropwise at rt under nitrogen atmosphere. The resulting mixture was stirred for 1 h at rt under nitrogen atmosphere. On completion, the reaction mixture was concentrated under reduced pressure. The residue was purified by reverse phase flash chromatography (Column: WelFlash™ C18-I, 20-40 um, 120 g; Eluent A: Water (plus 10 mmol/L NH₄HCO₃); Eluent B: ACN; Gradient: 40%-45% B in 20 min; Flow rate: 60 mL/min; Detector: 220/254 nm; desired fractions were collected at 43% B) and concentrated under reduced pressure to afford the title compound (140 mg, 84% yield) as a white solid. LC/MS (ESI, m/z): [(M+H)]⁺=397.2.

### Example 1.66. Preparation of 3-(3-methyl-2-oxo-4-(3-(piperidin-4-yloxy)cyclobutyl)-2,3-dihydro-1H-benzo[d]imidazol-1-yl)piperidine-2,6-dione (Intermediate BM)

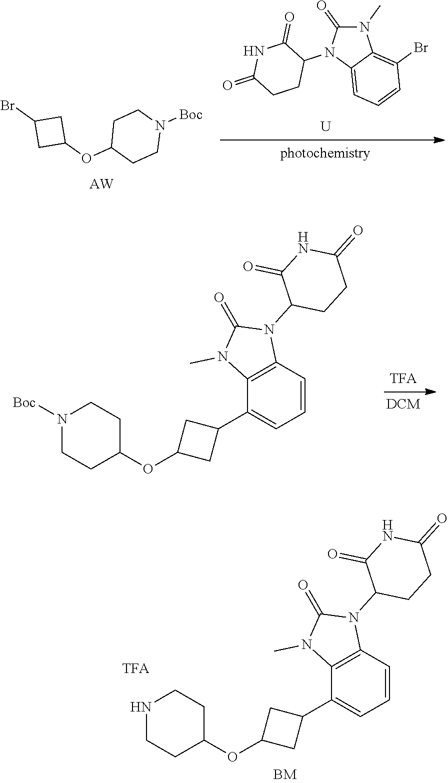

### Step 1—Tert-butyl 4-[3-[1-(2, 6-dioxo-3-piperidyl)-3-methyl-2-oxo-benzimidazol-4-yl]cyclobutoxy]piperidine-1-carboxylate

To a solution of 3-(4-bromo-3-methyl-2-oxo-2,3-dihydro-1H-benzo[d]imidazol-1-yl)piperidine-2,6-dione (3.7 g, 10.9 mmol, Intermediate U) in DME (140 mL) was added Ir[dF(CF₃)ppy]2(dtbpy)(PF₆) (122 mg, 109 umol), tert-butyl 4-(3-bromocyclobutoxy)piperidine-1-carboxylate (5.12 g, 15.3 mmol, Intermediate AW), Na₂CO₃ (2.32 g, 21.8 mmol), TTMSS (2.72 g, 10.9 mmol, 3.38 mL) and NiCl₂.dtbbpy (21.7 mg, 54.7 umol) at 25° C. The vial was sealed and placed under nitrogen atmosphere. The reaction was stirred and irradiated with a 34 W blue LED lamp (7 cm away), with cooling fan to keep the reaction temperature at 25° C. for 14 hr. On completion, the mixture was filtrated and concentrated by rotary evaporation. The residue was purified by column chromatography (SiO₂, EtOH/Ethyl acetate=1/20, Rf=0.9) followed by prep-HPLC (FA condition) to give the title compound (2.08 g, 82% yield) as white solid. ¹H NMR (400 MHz, DMSO) δ=11.08 (s, 1H), 7.19-6.94 (m, 3H), 5.36 (m, 1H), 4.25 (t, J=6.8 Hz, 1H), 4.15-3.96 (m, 1H), 3.74-3.44 (m, 6H), 3.08-2.83 (m, 3H), 2.78-2.55 (m, 4H), 2.45-2.31 (m, 1H), 2.13-1.94 (m, 2H), 1.77 (d, J=4.4 Hz, 2H), 1.48-1.21 (m, 11H); LC-MS (ESI+) m/z 413.2 (M+H-100)⁺.

### Step 2—3-(3-Methyl-2-oxo-4-(3-(piperidin-4-yloxy)cyclobutyl)-2,3-dihydro-1H-benzo[d]imidazol-1-yl)piperidine-2,6-dione Trifluoroacetate

To a stirred solution of tert-butyl 4-{3-[1-(2,6-dioxopiperidin-3-yl)-3-methyl-2-oxo-1,3-benzodiazol-4-yl]cyclobutoxy}piperidine-1-carboxylate (75.00 mg, 0.146 mmol) in DCM (6 mL) was added a solution of trifluoroacetaldehyde (1.2 mL) at rt. The resulting mixture was stirred for additional 1 h at rt. On completion, the reaction mixture was concentrated under reduced pressure. The residue was purified by trituration with diethyl ether (5 mL) to afford the title compound (73 mg) as an off-white solid. LC/MS (ESI, m/z): [(M+H)]⁺=413.2.

### Example 1.67. Preparation of tert-butyl 6-(5-ethynylpyrimidin-2-yl)-2,6-diazaspiro[3.3]heptane-2-carboxylate (Intermediate BN)

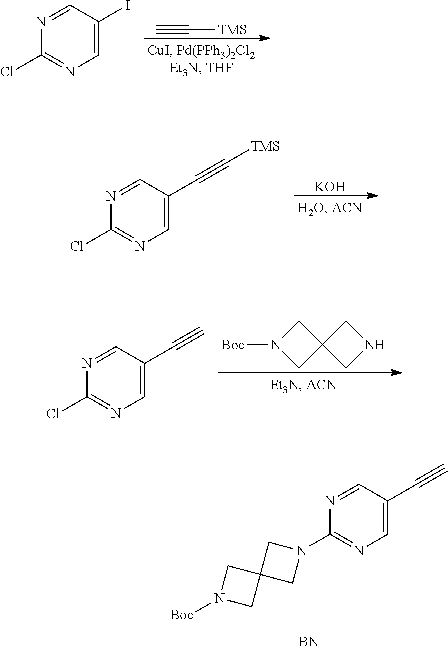

### Step 1—2-Chloro-5-((trimethylsilyl)ethynyl)pyrimidine

To a solution of 2-chloro-5-iodopyrimidine (8 g, 33 mmol) and ethynyltrimethylsilane (3.27 g, 33.3 mmol, 4.61 mL) in THF (80 mL) was added CuI (190.11 mg, 998.22 umol), Et₃N (6.73 g, 66.55 mmol, 9.26 mL) and Pd(PPh₃)₂Cl₂ (700.65 mg, 998.22 umol), then the mixture was stirred at 50° C. for 16 hrs under nitrogen atmosphere. On completion, the mixture was concentrated in vacuo to give the residue. The residue was purified by column chromatography (SiO₂, Petroleum ether/Ethyl acetate=1/0 to 5/1) to give the title compound (6.3 g, 90% yield) as brown solid. ¹H NMR (400 MHz, DMSO-d₆) δ=8.89 (s, 2H), 0.26 (s, 9H).

### Step 2—2-Chloro-5-ethynylpyrimidine

To a solution of 2-(2-chloropyrimidin-5-yl) ethynyl-trimethyl-silane (6.3 g, 30 mmol) in H₂O (15 mL) was added a mixture of KOH (4.36 g, 77.7 mmol) in ACN (23 mL) at 0° C., then the mixture was stirred at 0° C. for 15 min. On completion, the mixture was concentrated in vacuo to give the residue. The crude product was triturated with PE (50 mL) and filtered to give the title compound (3.4 g, 82% yield) as brown solid. ¹H NMR (400 MHz, CHLOROFORM-d) δ=8.70 (s, 2H), 3.46 (s, 1H)

### Step 3—Tert-butyl 6-(5-ethynylpyrimidin-2-yl)-2,6-diazaspiro[3.3]heptane-2-carboxylate

To a solution of 2-chloro-5-ethynylpyrimidine (1.5 g, 11 mmol) and tert-butyl 2, 6-diazaspiro[3.3]heptane-2-carboxylate (1.95 g, 9.84 mmol) in ACN (30 mL) was added Et₃N (2.99 g, 29.5 mmol, 4.11 mL) at 0° C. under nitrogen atmosphere, then the mixture was stirred at 50° C. for 1 hr. On completion, the mixture was concentrated in vacuo to give a residue. The residue was triturated with PE (100 mL) and filtered to give the title compound (3.8 g, 80% yield) as brown solid. ¹H NMR (400 MHz, DMSO-d₆) δ=8.44 (s, 2H), 4.29 (s, 1H), 4.19 (s, 4H), 4.03 (br s, 4H), 1.37 (s, 9H).

### Example 1.68. Preparation of 3-(5-((2-(2,6-diazaspiro[3.3]heptan-2-yl)pyrimidin-5-yl)ethynyl)-3-methyl-2-oxo-2,3-dihydro-1H-benzo[d]imidazol-1-yl)piperidine-2,6-dione (Intermediate BO)

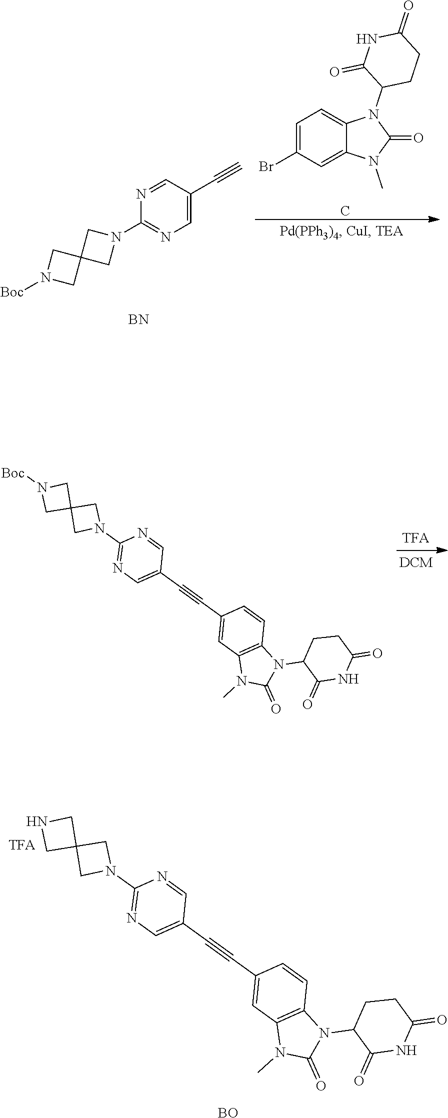

### Step 1—Tert-butyl 6-(5-((1-(2,6-dioxopiperidin-3-yl)-3-methyl-2-oxo-2,3-dihydro-1H-benzo[d]imidazol-5-yl)ethynyl)pyrimidin-2-yl)-2,6-diazaspiro[3.3]heptane-2-carboxylate

To a solution of tert-butyl 6-(5-ethynylpyrimidin-2-yl)-2, 6-diazaspiro[3.3]heptane-2-carboxylate (2.4 g, 5.0 mmol, Intermediate BN) and 3-(5-bromo-3-methyl-2-oxo-2, 3-dihydro-1H-benzo[d]imidazol-1-yl) piperidine-2, 6-dione (1.76 g, 5.20 mmol, Intermediate C) in DMSO (30 mL) was added CuI (94.35 mg, 495.4 μmol), Et₃N (2.51 g, 24.8 mmol, 3.45 mL) and Pd(PPh₃)₄ (572.48 mg, 495.41 μmol), then the mixture was stirred at 95° C. for 2 hr under nitrogen atmosphere. On completion, the mixture was filtered and the filtrate was concentrated in vacuo. The crude product was purified by reversed-phase HPLC (0.1% FA condition). The resulting crude product was triturated with PE:EA (3:1) (50 mL×2) to give the title compound (551.76 mg, 10% yield) as yellow solid. ¹H NMR (400 MHz, DMSO-d₆) δ=11.13 (s, 1H), 8.51 (s, 2H), 7.38 (s, 1H), 7.18 (s, 2H), 5.40 (dd, J=5.2, 12.8 Hz, 1H), 4.22 (s, 4H), 4.09-4.00 (m, 4H), 3.36 (s, 3H), 2.97-2.83 (m, 1H), 2.79-2.63 (m, 2H), 2.04 (br s, 1H), 1.38 (s, 9H).

### Step 1—3-(5-((2-(2,6-Diazaspiro[3.3]heptan-2-yl)pyrimidin-5-yl)ethynyl)-3-methyl-2-oxo-2,3-dihydro-1H-benzo[d]imidazol-1-yl)piperidine-2,6-dione Trifluoroacetate

To a stirred solution of tert-butyl 6-(5-{2-[1-(2,6-dioxopiperidin-3-yl)-3-methyl-2-oxo-1,3-benzodiazol-5-yl]ethynyl}pyrimidin-2-yl)-2,6-diazaspiro[3.3]heptane-2-carboxylate (50.06 mg, 0.090 mmol) in DCM (2 mL) was added TFA (0.4 mL) dropwise at rt. The resulting mixture was stirred for additional 1 h at rt. On completion, the reaction mixture was concentrated under reduced pressure. The residue was purified by trituration with diethyl ether (5 mL) to afford the title compound (49.00 mg) as a yellow solid. LC/MS (ESI, m/z): [(M+H)]⁺=458.1.

### Example 1.69. Preparation of N-cyclopropyl-6-{[3-(5-formylpyridin-2-yl)phenyl]amino}imidazo[1,2-b]pyridazine-3-carboxamide (Intermediate BP)

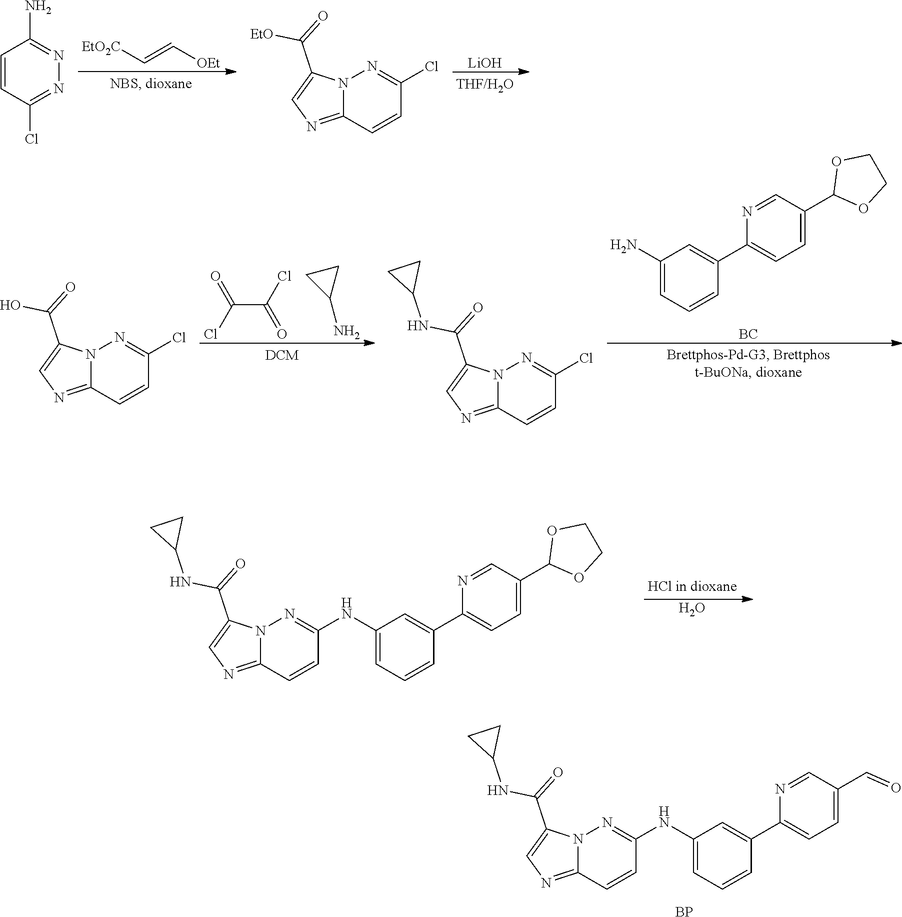

### Step 1—Ethyl 6-chloroimidazo[1,2-b]pyridazine-3-carboxylate

To a solution of ethyl 3-ethoxy-2-propenoate (11.13 g, 77.19 mmol) in 1,4-dioxane (100 mL) and water (50 mL) was added NBS (15.11 g, 84.91 mmol) in portions at −10° C. under nitrogen atmosphere followed by the addition of 3-Cl-6-pyridazinamine (10 g, 77.190 mmol) at rt. The resulting mixture was stirred for additional 1 h at 80° C. On completion, the resulting mixture was concentrated under reduced pressure. The residue was purified by silica gel column chromatography, eluted with PE/EA (1:1) to afford the title compound (10 g, 57% yield) as a light yellow solid. LC/MS (ESI, m/z): [(M+H)]⁺=226.1.

### Step 2—6-Chloroimidazo[1,2-b]pyridazine-3-carboxylic Acid

To a solution of ethyl 6-chloroimidazo[1,2-b]pyridazine-3-carboxylate (10 g, 44 mmol) in THF (100 mL) and water (100 mL) was added lithiumol (10.61 g, 443.2 mmol). The reaction mixture was stirred for 1 h at rt. On completion, the reaction mixture was concentrated under reduced pressure. The mixture was acidified to pH 3 with aq. 2N HCl. The precipitated solids were collected by filtration and washed with water. The resulting solid was dried by lyophilization to afford the title compound (8.3 g, 95% yield) as an off-white solid. LC/MS (ESI, m/z): [(M+H)]⁺=198.0.

### Step 3—6-Chloro-N-cyclopropylimidazo[1,2-b]pyridazine-3-carboxamide

To a stirred solution of 6-chloroimidazo[1,2-b]pyridazine-3-carboxylic acid (4.2 g, 21 mmol) in DCM (500 mL) was added oxalyl chloride (4.05 g, 31.9 mmol) dropwise within 2 min, followed by addition of DMF (0.16 mL, 2.1 mmol) dropwise at 0° C. under nitrogen atmosphere. The resulting mixture was stirred for 30 min at rt under nitrogen atmosphere. Once acyl chloride generated, the resulting mixture was concentrated under reduced pressure. The residue was re-dissolved in THF (500 mL). Then to the above mixture was added into a solution of TEA (8.83 mL, 63.8 mmol) and aminocyclopropane (2.43 g, 42.5 mmol) in THF (200 mL) dropwise over 15 min at 0° C. The resulting mixture was stirred for additional 2 h at rt. On completion, the reaction mixture was concentrated under reduced pressure. The residue was purified by silica gel column chromatography, eluted with PE/EA (1:1) to afford the title compound (4 g, 80% yield) as a white solid. LC/MS (ESI, m/z): [(M+H)]⁺=237.1.

### Step 4—N-cyclopropyl-6-({3-[5-(1,3-dioxolan-2-yl)pyridin-2-yl]phenyl}amino)imidazo[1,2-b]pyridazine-3-carboxamide

To a stirred mixture of 6-chloro-N-cyclopropylimidazo[1,2-b]pyridazine-3-carboxamide (6 g, 25 mmol) and 3-[5-(1,3-dioxolan-2-yl)pyridin-2-yl]aniline (6.14 g, 25.3 mmol, Intermediate BC) in 1,4-dioxane (100 mL) were added K₂CO₃ (7.01 g, 50.7 mmol), Pd(OAc)₂ (0.57 g, 2.5 mmol) and BrettPhos (1.36 g, 2.53 mmol) at rt under nitrogen atmosphere. The resulting mixture was stirred for 2 h at 110° C. On completion, the reaction mixture was cooled to rt and poured into the ice/water (100 mL). The precipitated solids were collected by filtration and washed with 1,4-dioxane (5×10 mL). The solid was dried under reduced pressure to afford the title compound (7 g, 62% yield) as a brown solid. LC/MS (ESI, m/z): [(M+H)]⁺=443.2.

### Step 5—N-cyclopropyl-6-{[3-(5-formylpyridin-2-yl)phenyl]amino}imidazo[1,2-b]pyridazine-3-carboxamide

To a stirred solution of N-cyclopropyl-6-({3-[5-(1,3-dioxolan-2-yl)pyridin-2-yl]phenyl}amino)imidazo[1,2-b]pyridazine-3-carboxamide (450 mg, 1.02 mmol) in H₂O (5 mL) was added 4 M HCl (gas) in 1,4-dioxane (5 mL) dropwise at rt. The resulting mixture was stirred for 2 h at 50° C. under nitrogen atmosphere. On completion, the reaction mixture was cooled to rt and basified to pH 8 with saturated NaHCO₃ (aq.). The precipitated solids were collected by filtration, washed with water (3×10 mL) and dried under reduced pressure to afford the title compound (350 mg, 86% yield) as a white solid. LC/MS (ESI, m/z): [(M+H)]⁺=399.2.

### Example 1.70. Preparation of tert-butyl 3-oxo-4-[5-(4,4,5,5-tetramethyl-1,3,2-dioxaborolan-2-yl)pyridin-2-yl]piperazine-1-carboxylate (Intermediate BQ)

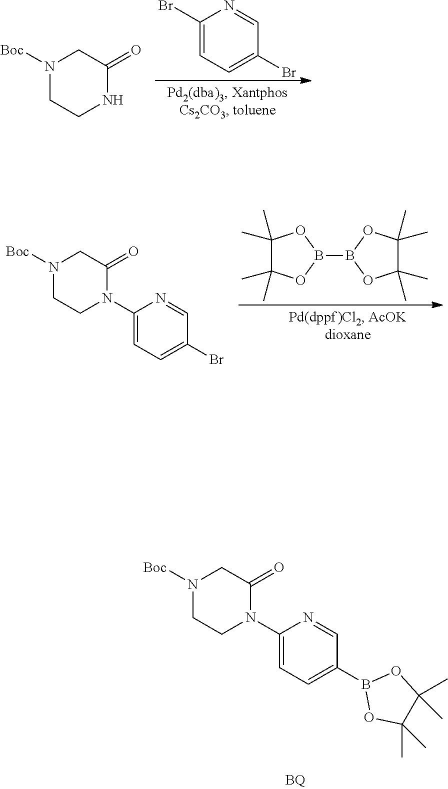

### Step 1—Tert-butyl 4-(5-bromopyridin-2-yl)-3-oxopiperazine-1-carboxylate

To a stirred mixture of tert-butyl 3-oxopiperazine-1-carboxylate (10 g, 50 mmol) and 2,5-dibromopyridine (11.83 g, 49.94 mmol) in toluene (150 mL) were added Pd₂(dba)₃ (4.57 g, 4.99 mmol) and XantPhos (2.89 g, 4.99 mmol) and Cs₂CO₃ (32.54 g, 99.88 mmol) in turns at rt under nitrogen atmosphere. The resulting mixture was stirred for 16 h at 100° C. under nitrogen atmosphere. On completion, the reaction mixture was cooled to rt and diluted with water (400 mL), then extracted with EtOAc (3×200 mL). The combined organic layers were washed with brine (3×200 mL), and dried over anhydrous Na₂SO₄. After filtration, the filtrate was concentrated under reduced pressure. The residue was purified by silica gel column chromatography, eluted with PE/EA (5:1) to afford the title compound (4.6 g, 26% yield) as a brown oil. LC/MS (ESI, m/z): [(M+H)]⁺=356.1, 358.1.

### Step 2—Tert-butyl 3-oxo-4-[5-(4,4,5,5-tetramethyl-1,3,2-dioxaborolan-2-yl)pyridin-2-yl]piperazine-1-carboxylate

To a stirred mixture of tert-butyl 4-(5-bromopyridin-2-yl)-3-oxopiperazine-1-carboxylate (4.6 g, 13 mmol) and bis(pinacolato)diboron (4.92 g, 19.4 mmol) in dioxane (50 mL) were added Pd(dppf)Cl₂·CH₂Cl₂ (1.05 g, 1.29 mmol) and AcOK (2.53 g, 25.8 mmol) in turns at rt under nitrogen atmosphere. The resulting mixture was stirred for 2 h at 100° C. under nitrogen atmosphere. On completion, the reaction mixture was cooled to rt and concentrated under reduced pressure. The residue was purified by silica gel column chromatography, eluted with PE/EA (1:1) to afford the title compound (5.2 g, 100% yield) as a brown solid. LC/MS (ESI, m/z): [(M+H)]⁺=404.2.

### Example 1.71. Preparation of 3-{3-methyl-2-oxo-5-[6-(2-oxopiperazin-1-yl)pyridin-3-yl]-1,3-benzodiazol-1-yl}piperidine-2,6-dione (Intermediate BR)

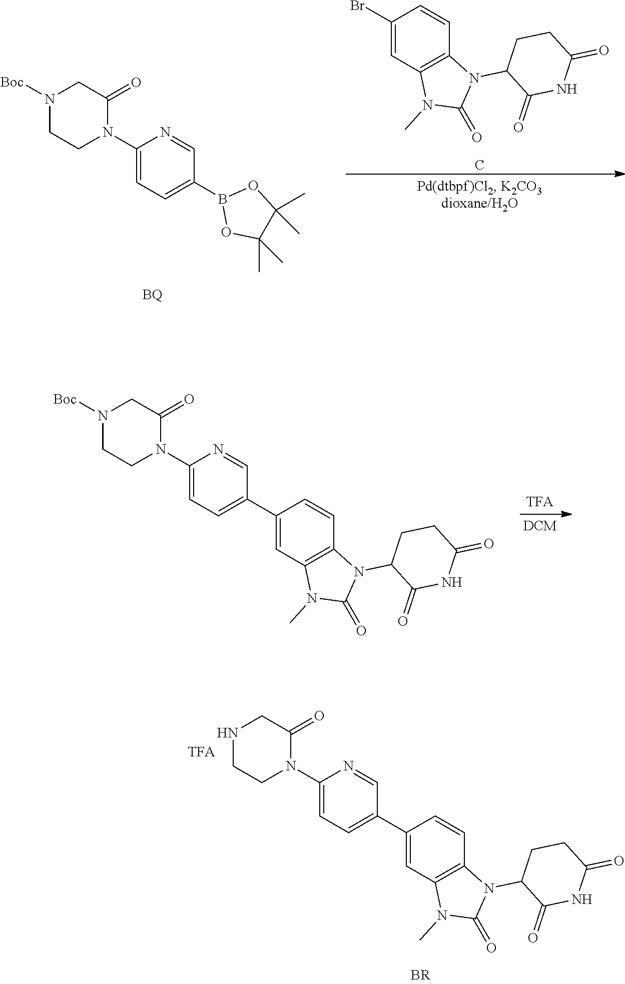

### Step 1—Tert-butyl 4-(5-(1-(2,6-dioxopiperidin-3-yl)-3-methyl-2-oxo-2,3-dihydro-1H-benzo[d]imidazol-5-yl)pyridin-2-yl)-3-oxopiperazine-1-carboxylate

To a stirred mixture of tert-butyl 3-oxo-4-(5-(4,4,5,5-tetramethyl-1,3,2-dioxaborolan-2-yl)pyridin-2-yl)piperazine-1-carboxylate (3 g, 8 mmol, Intermediate BQ) and 3-(5-bromo-3-methyl-2-oxo-1,3-benzodiazol-1-yl)piperidine-2,6-dione (3.80 g, 11.2 mmol) in dioxane (60 mL) and H₂O (12 mL) were added Pd(DtBPF)Cl₂ (0.49 g, 0.75 mmol) and K₂CO₃ (2.07 g, 15.0 mmol) in portions at rt under nitrogen atmosphere. The resulting mixture was stirred for 2 h at 80° C. under nitrogen atmosphere. On completion, the mixture was cooled to rt and diluted with water (200 mL), then extracted with EtOAc (3×200 mL). The combined organic layers were washed with brine (3×200 mL), and dried over anhydrous Na₂SO₄. After filtration, the filtrate was concentrated under reduced pressure. The residue was purified by silica gel column chromatography, eluted with PE/EA (1:1) to afford the title compound (3.3 g, 83% yield) as a brown oil. LC/MS (ESI, m/z): [(M+H)]⁺=535.2.

### Step 4—3-{3-Methyl-2-oxo-5-[6-(2-oxopiperazin-1-yl)pyridin-3-yl]-1,3-benzodiazol-1-yl}piperidine-2,6-dione Trifluoroacetate

To a stirred solution of tert-butyl 4-{5-[1-(2,6-dioxopiperidin-3-yl)-3-methyl-2-oxo-1,3-benzodiazol-5-yl]pyridin-2-yl}-3-oxopiperazine-1-carboxylate (1 g, 2 mmol) in DCM (10 mL) was added TFA (2 mL) dropwise at 0° C. under nitrogen atmosphere. The resulting mixture was stirred for 2 h at 25° C. under nitrogen atmosphere. On completion, the mixture was concentrated under reduced pressure. The residue was purified by trituration with Et₂O (60 mL) to afford the title compound (1 g) as an off-white solid. LC/MS (ESI, m/z): [(M+H)]⁺=435.1.

### Example 1.72. Preparation of 3-{3-[(3-{[(1R,2S)-2-fluorocyclopropyl]carbamoyl}-8-(methylamino)imidazo[1,2-b]pyridazin-6-yl)amino]-2-oxopyridin-1-yl}benzoic Acid (Intermediate BS)

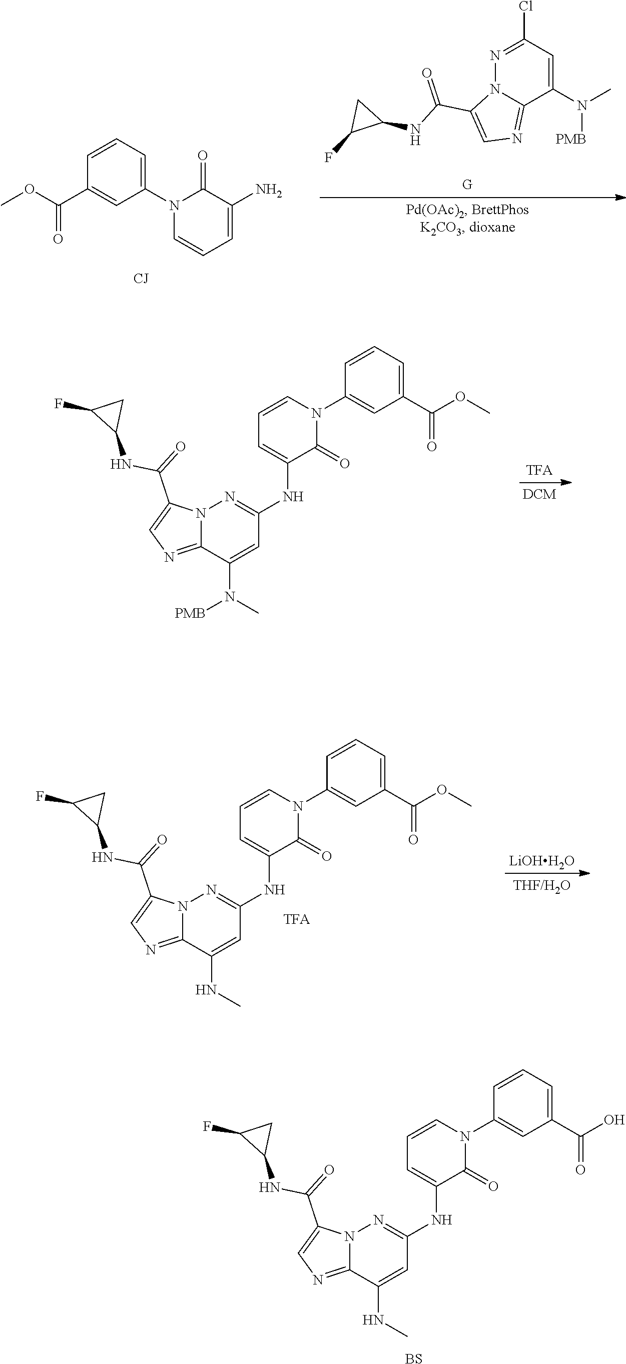

### Step 1—Methyl 3-{3-[(3-{[(1R,2S)-2-fluorocyclopropyl]carbamoyl}-8-{[(4-methoxyphenyl)methyl](methyl)amino}imidazo[1,2-b]pyridazin-6-yl)amino]-2-oxopyridin-1-yl}benzoate

To a stirred solution of methyl 3-(3-amino-2-oxopyridin-1-yl)benzoate (2.5 g, 10 mmol, Intermediate CJ) and 6-chloro-N-[(1R,2S)-2-fluorocyclopropyl]-8-{[(4-methoxyphenyl)methyl](methyl)amino}imidazo[1,2-b]pyridazine-3-carboxamide (4.55 g, 11.3 mmol, Intermediate G) in dioxane (40 mL) were added Pd(OAc)₂ (0.23 g, 1.0 mmol), BrettPhos (0.55 g, 1.0 mmol) and K₂CO₃ (2.83 g, 20.5 mmol) in turns at rt under N₂ atmosphere. The resulting mixture was stirred for 2 h at 100° C. under N₂ atmosphere. On completion, the reaction mixture was filtered, the filter cake was washed with DCM (3×30 mL). The filtrate was concentrated under reduced pressure. The residue was diluted with water (100 mL) and the mixture was extracted with CH₂Cl₂ (3×30 mL). The combined organic layers were washed with brine (3×50 mL), and dried over anhydrous Na₂SO₄. After filtration, the filtrate was concentrated under reduced pressure. The residue was purified by silica gel column chromatography, eluted with DCM/MeOH (10:1) to afford the title compound (6 g, 96% yield) as a white solid. LC/MS (ESI, m/z): [(M+H)]⁺=612.4.

### Step 2—Methyl 3-{3-[(3-{[(1R,2S)-2-fluorocyclopropyl]carbamoyl}-8-(methylamino)imidazo[1,2-b]pyridazin-6-yl)amino]-2-oxopyridin-1-yl}benzoate

To a stirred solution of methyl 3-{3-[(3-{[(1R,2S)-2-fluorocyclopropyl]carbamoyl}-8-{[(4-methoxyphenyl)methyl](methyl)amino}imidazo[1,2-b]pyridazin-6-yl)amino]-2-oxopyridin-1-yl}benzoate (6 g, 10 mmol) in DCM (60 mL) was added TFA (20 mL) at rt. The resulting mixture was stirred for 2 h at rt. On completion, the reaction mixture was concentrated under reduced pressure. The residue was triturated with Et₂O to afford the title compound (3.5 g) as a light yellow solid. LC/MS (ESI, m/z): [(M+H)]⁺=492.3.

### Step 3—3-{3-[(3-{[(1R,2S)-2-fluorocyclopropyl]carbamoyl}-8-(methylamino)imidazo[1,2-b]pyridazin-6-yl)amino]-2-oxopyridin-1-yl}benzoic Acid

To a stirred solution of methyl 3-{3-[(3-{[(1R,2S)-2-fluorocyclopropyl]carbamoyl}-8-(methylamino)imidazo[1,2-b]pyridazin-6-yl)amino]-2-oxopyridin-1-yl}benzoate (3.5 g, 7.1 mmol) in THF (20 mL) and H₂O (20 mL) was added LiOH (1.71 g, 71.2 mmol) at 0° C. The resulting mixture was stirred for 2 h at rt. On completion, the reaction mixture was concentrated under reduced pressure. The mixture/residue was acidified to pH 3 with HCl (2 mol/L). The precipitated solids were collected by filtration and washed with H₂O (3×30 mL) to afford the title compound (2.4 g, 71% yield) as a white solid. ¹H NMR (400 MHz, DMSO-d₆) δ 13.16 (s, 1H), 8.68-8.64 (m, 1H), 8.62 (s, 1H), 8.08-8.02 (m, 1H), 8.03-7.96 (m, 2H), 7.92 (s, 1H), 7.81-7.76 (m, 1H), 7.73-7.67 (m, 1H), 7.54 (m, 1H), 7.37 (dd, J=7.0, 1.8 Hz, 1H), 6.57-6.09 (m, 2H), 5.00-4.77 (m, 1H), 3.05-2.94 (m, 1H), 2.87 (d, J=4.7 Hz, 3H), 1.32-1.14 (m, 1H), 1.07-0.91 (m, 1H). LC/MS (ESI, m/z): [(M+H)]⁺=478.2.

### Example 1.73. Preparation of 3-[5-(6-aminohexyl)-3-methyl-2-oxo-1,3-benzodiazol-1-yl]piperidine-2,6-dione (Intermediate BT)

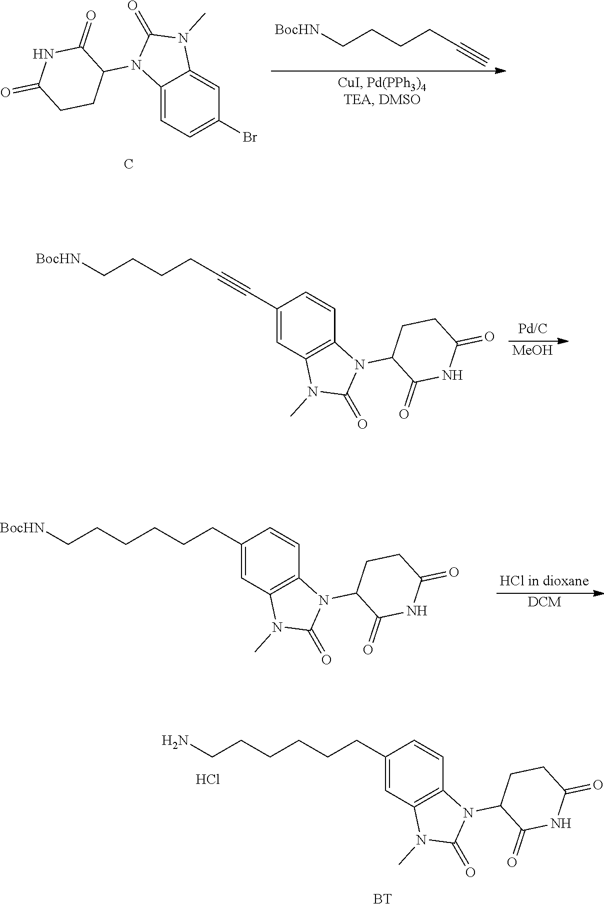

### Step 1—Tert-butyl N-[6-[1-(2,6-dioxopiperidin-3-yl)-3-methyl-2-oxo-1,3-benzodiazol-5-yl]hex-5-yn-1-yl]carbamate

To a stirred solution of 3-(5-bromo-3-methyl-2-oxo-1,3-benzodiazol-1-yl)piperidine-2,6-dione (8.00 g, 23.7 mmol, Intermediate C) and tert-butyl N-(hex-5-yn-1-yl)carbamate (9.33 g, 47.3 mmol, CAS #151978-58-4) in DMSO (40.00 mL) and TEA (20.00 mL) were added Pd(PPh₃)₄ (2.73 g, 2.37 mmol) and CuI (450.55 mg, 2.366 mmol) in portions at rt under nitrogen atmosphere. The resulting mixture was stirred for 3 h at 80° C. under nitrogen atmosphere. On completion, the reaction mixture was diluted with EtOAc (800 mL). The resulting mixture was washed with water (4×400 mL). The resulting mixture was concentrated under reduced pressure. The residue was purified by silica gel column chromatography, eluted with PE/EtOAc (1:2) to afford the title compound (7.2 g, 67% yield) as a yellow solid. LC/MS (ESI, m/z): [(M+1)]⁺=455.2.

### Step 2—Tert-butyl N-[6-[1-(2,6-dioxopiperidin-3-yl)-3-methyl-2-oxo-1,3-benzodiazol-5-yl]hexyl]carbamate

To a stirred solution of tert-butyl N-[6-[1-(2,6-dioxopiperidin-3-yl)-3-methyl-2-oxo-1,3-benzodiazol-5-yl]hex-5-yn-1-yl]carbamate (6.00 g, 13.2 mmol) in MeOH (500.00 mL) was added Pd/C (1.97 g, 1.85 mmol, 10 wt %) in portions at rt under nitrogen atmosphere. The reaction system was degassed under vacuum and purged with H₂ several times, then it was hydrogenated under H₂ balloon (1 atm) at rt for 48 h. After completion of the reaction, Pd/C was filtered through celite. The filter cake was washed with MeOH (3×100 mL). The corresponding filtrate was concentrated under reduced pressure. The residue was purified by reverse flash chromatography (column, C18 silica gel; mobile phase A: water (0.1% FA), mobile phase B: ACN, 60% to 80% gradient in 25 min; detector, UV 254 nm; the fractions was collected at 70% B) and concentrated under vacuum to afford the title compound (5 g, 83% yield) as a white solid. LC/MS (ESI, m/z): [(M+1)]⁺=459.2.

### Step 3—3-[5-(6-aminohexyl)-3-methyl-2-oxo-1,3-benzodiazol-1-yl]piperidine-2,6-dione Hydrochloride

To a stirred solution of tert-butyl N-[6-[1-(2,6-dioxopiperidin-3-yl)-3-methyl-2-oxo-1,3-benzodiazol-5-yl]hexyl]carbamate (4.8 g, 11 mmol) in DCM (20 mL) was added 4 M HCl (gas) in 1,4-dioxane (20 mL) dropwise at rt under nitrogen atmosphere. The resulting mixture was stirred at rt for 2 h. After completion, the reaction mixture was concentrated under reduced pressure. The residue was triturated with Et₂O to afford the title compound (4 g, 97% yield) as a yellow solid. ¹H NMR (400 MHz, Methanol-d₄) δ 7.05-7.01 (m, 2H), 6.99-6.95 (m, 1H), 5.34 (dd, J=12.4, 5.4 Hz, 1H), 3.43 (s, 3H), 2.97-2.88 (m, 3H), 2.87-2.77 (m, 2H), 2.72 (t, J=7.6 Hz, 2H), 2.23-2.13 (m, 1H), 1.74-1.63 (m, 4H), 1.49-1.38 (m, 4H). LC/MS (ESI, m/z): [(M+1)]⁺=359.2.

### Example 1.74. Preparation of 2-[(4-{[3-(5-fluoropyrimidin-2-yl)-2-methoxyphenyl]amino}-5-(methylcarbamoyl)pyridin-2-yl)amino]pyridine-4-carboxylic Acid (Intermediate BU)

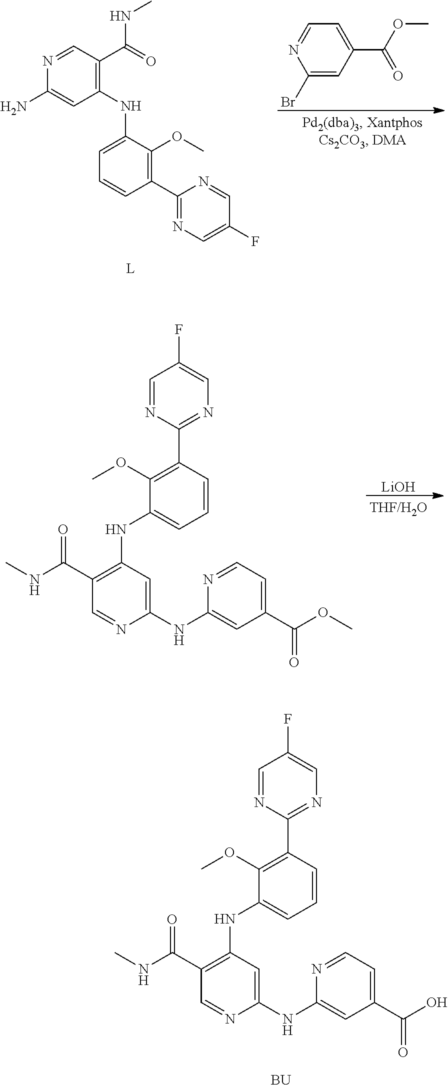

### Step 1—Methyl 2-[(4-{[3-(5-fluoropyrimidin-2-yl)-2-methoxyphenyl]amino}-5-(methylcarbamoyl)pyridin-2-yl)amino]pyridine-4-carboxylate

To a stirred mixture of 6-amino-4-{[3-(5-fluoropyrimidin-2-yl)-2-methoxyphenyl]amino}-N-methylpyridine-3-carboxamide (1 g, 3 mmol, Intermediate L) and methyl 2-bromopyridine-4-carboxylate (0.88 g, 4.1 mmol) in DMA were added Pd₂(dba)₃ (0.25 g, 0.27 mmol), XantPhos (0.16 g, 0.27 mmol) and Cs₂CO₃ (1.77 g, 5.43 mmol) in turns at rt under N₂ atmosphere. The resulting mixture was stirred for 3 h at 120° C. under N₂ atmosphere. The residue was purified by reverse flash chromatography (column, C18 silica gel; mobile phase A: water (10 mmol/L NH₄HCO₃), mobile phase B: CAN, 10% to 50% gradient in 40 min; detector, UV 254 nm; the fractions were collected at 45%) and concentrated under reduced pressure to afford the title compound (310 mg, 23% yield) as a yellow solid. LC/MS (ESI, m/z): [(M+H)]⁺=504.2.

### Step 2—2-[(4-{[3-(5-fluoropyrimidin-2-yl)-2-methoxyphenyl]amino}-5-(methylcarbamoyl)pyridin-2-yl)amino]pyridine-4-carboxylic Acid

To a stirred solution of methyl 2-[(4-{[3-(5-fluoropyrimidin-2-yl)-2-methoxyphenyl]amino}-5-(methylcarbamoyl)pyridin-2-yl)amino]pyridine-4-carboxylate (300 mg, 0.6 mmol) in water (6 mL) and THF (6 mL) was added LiOH (142.70 mg, 5.960 mmol) at 0° C. The resulting mixture was stirred for 2 h at rt. On completion, the reaction mixture was concentrated under reduced pressure. The mixture was acidified to pH 3 with HCl (aq. 2 mol/L). The precipitated solids were collected by filtration and washed with water to afford the title compound (260 mg, 89% yield) as a light yellow solid. LC/MS (ESI, m/z): [(M+H)]⁺=490.2.

### Example 1.75. Preparation of 3-(4-(6-Aminohexyl)-3-methyl-2-oxo-2,3-dihydro-1H-benzo[d]imidazol-1-yl)piperidine-2,6-dione Hydrochloride (Intermediate BV)

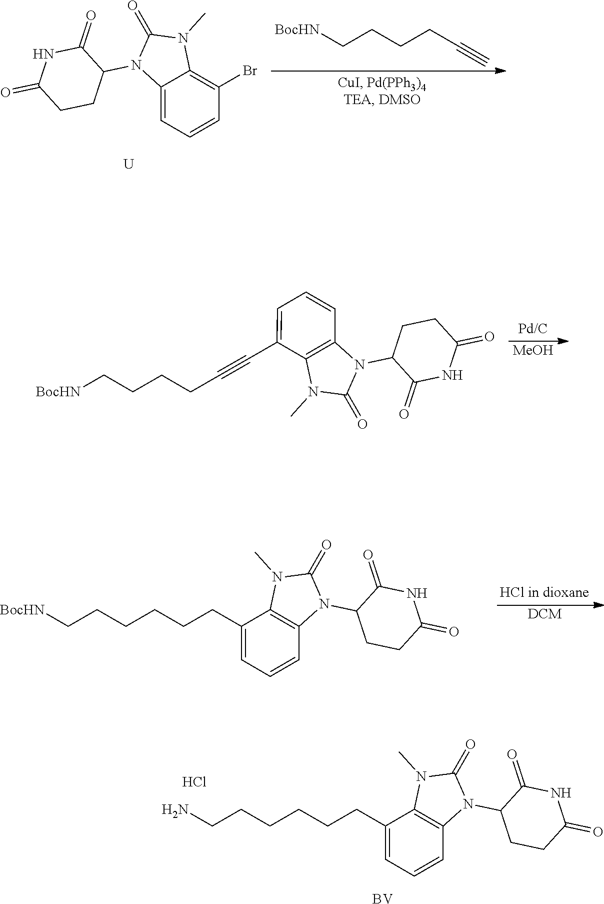

### Step 1—Tert-butyl N-[6-[1-(2,6-dioxopiperidin-3-yl)-3-methyl-2-oxo-1,3-benzodiazol-4-yl]hex-5-yn-1-yl]carbamate

To a stirred mixture of 3-(4-bromo-3-methyl-2-oxo-1,3-benzodiazol-1-yl)piperidine-2,6-dione (8.00 g, 23.7 mmol, Intermediate U) and tert-butyl N-(hex-5-yn-1-yl)carbamate (7.00 g, 35.5 mmol, CAS #151978-58-4) in DMA (40.00 mL) and TEA (20.00 mL) were added Pd(PPh₃)₄ (2.73 g, 2.37 mmol) and CuI (450.55 mg, 2.366 mmol) in portions at rt under nitrogen atmosphere. The resulting mixture was stirred for 3 h at 80° C. under nitrogen atmosphere. On completion, the mixture was diluted with water (100 mL) and extracted with EtOAc (3×75 mL). The combined organic layers were washed with brine (3×50 mL), dried over anhydrous Na₂SO₄. After filtration, the filtrate was concentrated under reduced pressure. The residue product was purified by reverse phase flash (Column: Spherical C18, 20˜40 um, 330 g; Mobile Phase A: Water (0.05% FA), Mobile Phase B: ACN; Flow rate: 80 mL/min; Gradient (B %): 5%˜5%, 8 min; 40%˜60%, 30 min; 60%˜95%; 0 min; 95%, 5 min; Detector: 254 nm; Rt: 23.4 min.) to afford the title compound (4.5 g, 42% yield) as a yellow solid. LC/MS (ESI, m/z): [(M+1)]⁺=455.2.

### Step 2—Tert-butyl N-[7-[1-(2,6-dioxopiperidin-3-yl)-3-methyl-2-oxo-1,3-benzodiazol-4-yl]heptyl]carbamate

To a stirred mixture of tert-butyl N-[6-[1-(2,6-dioxopiperidin-3-yl)-3-methyl-2-oxo-1,3-benzodiazol-4-yl]hex-5-yn-1-yl]carbamate (5.50 g, 12.1 mmol) in MeOH (500.00 mL) and DCM (50.00 mL) was added Pd/C (1.29 g, 12.1 mmol, 10 wt %) at rt under hydrogen atmosphere. The reaction system was degassed under vacuum and purged with H₂ several times, then it was hydrogenated under H₂ balloon (1 atm) at rt for 4 h. After completion of the reaction, Pd/C was filtered off through celite. The filter cake was washed with MeOH (3×100 mL). The corresponding filtrate was concentrated under reduced pressure to afford the title compound (5 g, 90% yield) as a white solid. LC/MS (ESI, m/z): [(M+1)]⁺=459.2.

### Step 3—3-[4-(6-Aminohexyl)-3-methyl-2-oxo-1,3-benzodiazol-1-yl]piperidine-2,6-dione Hydrochloride

To a stirred solution of tert-butyl N-[7-[1-(2,6-dioxopiperidin-3-yl)-3-methyl-2-oxo-1,3-benzodiazol-4-yl]heptyl]carbamate (6.00 g, 12.7 mmol) in DCM (50.00 mL) was added 4 M HCl (gas) in 1,4-dioxane (20 mL) dropwise at rt. The resulting mixture was stirred for 2 h at rt under nitrogen atmosphere. On completion, the reaction mixture was concentrated under vacuum. The residue was triturated with Et₂O to afford the title compound (4.5 g, 90% yield) as a white solid. ¹H NMR (400 MHz, DMSO-d₆) δ 11.10 (s, 1H), 7.96 (s, broad, 3H), 7.00-6.94 (m, 2H), 6.89-6.85 (m, 1H), 5.38 (dd, J=12.6, 5.4 Hz, 1H), 3.56 (s, 3H), 2.99-2.84 (m, 3H), 2.82-2.59 (m, 4H), 2.04-1.97 (m, 1H), 1.64-1.54 (m, 4H), 1.43-1.36 (m, 4H). LC/MS (ESI, m/z): [(M+1)]⁺=359.2.

### Example 1.76. Preparation of 3-[3-methyl-2-oxo-4-(piperazin-1-yl)-1,3-benzodiazol-1-yl]piperidine-2,6-dione (Intermediate BW)

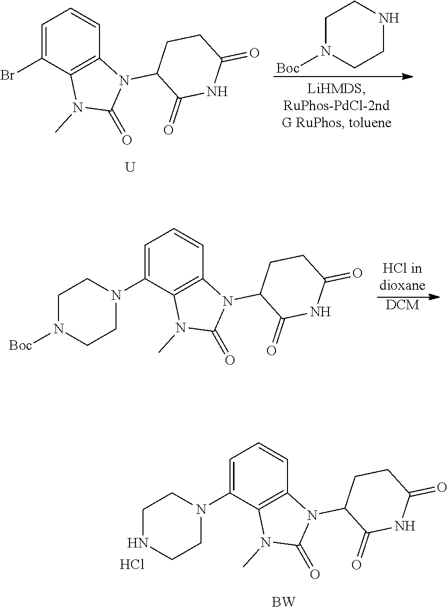

### Step 1—Tert-butyl 4-[1-(2,6-dioxopiperidin-3-yl)-3-methyl-2-oxo-1,3-benzodiazol-4-yl]piperazine-1-carboxylate

To a stirred mixture of 3-(4-bromo-3-methyl-2-oxo-1,3-benzodiazol-1-yl)piperidine-2,6-dione (2 g, 6 mmol, Intermediate U) and tert-butyl piperazine-1-carboxylate (2.20 g, 11.8 mmol) in methylbenzene (20 mL) were added RuPhos-PdCl-2nd G (0.92 g, 1.2 mmol), RuPhos (0.55 g, 1.2 mmol) and LiHMDS (35.84 mL, 35.48 mmol) at rt under nitrogen atmosphere. The resulting mixture was stirred for 1 h at 80° C. under nitrogen atmosphere. The residue was basified to pH 5 with conc. HCl, then the mixture was filtrated. The filtrate was concentrated under reduced pressure. The crude product was purified by reverse phase flash (Column: WelFlash™ C18-I, 20-40 um, 330 g; Eluent A: Water (10 mmol/L NH₄HCO₃); Eluent B: ACN; Gradient: 20%-55% B in 35 min; Flow rate: 80 mL/min; Detector: 254 nm; desired fractions were collected at 35% B) and concentrated under reduced pressure to afford the title compound (760 mg, 29% yield) as a yellow solid. LC/MS (ESI, m/z): [(M+1)]⁺=444.2.

### Step 2—3-[3-Methyl-2-oxo-4-(piperazin-1-yl)-1,3-benzodiazol-1-yl]piperidine-2,6-dione Hydrochloride

To a stirred mixture of tert-butyl 4-[1-(2,6-dioxopiperidin-3-yl)-3-methyl-2-oxo-1,3-benzodiazol-4-yl]piperazine-1-carboxylate (760 mg, 1.7 mmol) in DCM (5 mL) was added 4 M HCl (gas) in 1,4-dioxane (5 mL) at rt under nitrogen atmosphere. The resulting mixture was stirred for 1 h at rt under nitrogen atmosphere. On completion, the reaction mixture was concentrated under reduced pressure. The residue was triturated with Et₂O to afford the title compound (650 mg, quantitative yield) as a yellow solid. LC/MS (ESI, m/z): [(M+1)]⁺=344.2.

### Example 1.77. Preparation of 4-{[3-(5-fluoropyrimidin-2-yl)-2-methoxyphenyl]amino}-6-[(4-formylpyridin-2-yl)amino]-N-methylpyridine-3-carboxamide (Intermediate BX)

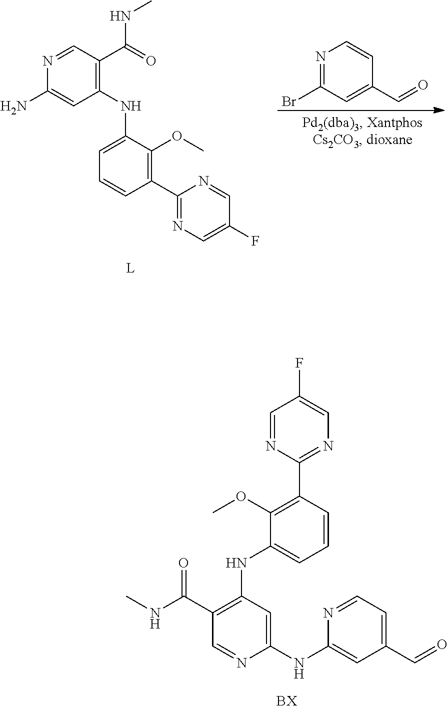 [SMILES: CNC(=O)c1cnc(N)cc1Nc1cccc(-c2ncc(F)cn2)c1OC ; O=Cc1ccnc(Br)c1 ; CNC(=O)c1cnc(Nc2cc(C=O)ccn2)cc1Nc1cccc(-c2ncc(F)cn2)c1OC]

To a stirred mixture of 6-amino-4-{[3-(5-fluoropyrimidin-2-yl)-2-methoxyphenyl]amino}-N-methylpyridine-3-carboxamide (250 mg, 0.679 mmol, Intermediate L) and 2-bromopyridine-4-carbaldehyde (150 mg, 0.815 mmol) in dioxane (5 mL) were added Pd₂(dba)₃ (61 mg, 0.068 mmol) and XantPhos (43 mg, 0.068 mmol) and Cs₂CO₃ (442 mg, 1.36 mmol) at rt under nitrogen atmosphere. The resulting mixture was stirred for 1 h at 105° C. under nitrogen atmosphere. On completion, the mixture was allowed to cool down to rt. The crude product was purified by reverse phase flash (Column: WelFlash™ C18-I, 20-40 um, 80 g; Eluent A: Water (10 mmol/L NH₄HCO₃); Eluent B: ACN; Gradient: 25%-55% B in 25 min; Flow rate: 60 mL/min; Detector: 254 nm; desired fractions were collected at 43% B) and concentrated under reduced pressure to afford the title compound (71 mg, 22% yield) as a yellow solid. LC/MS (ESI, m/z): [(M+H)]⁺=474.2.

### Example 1.78. Preparation of tert-butyl (3-(3-bromocyclobutoxy) propyl)(methyl) carbamate (Intermediate BY)

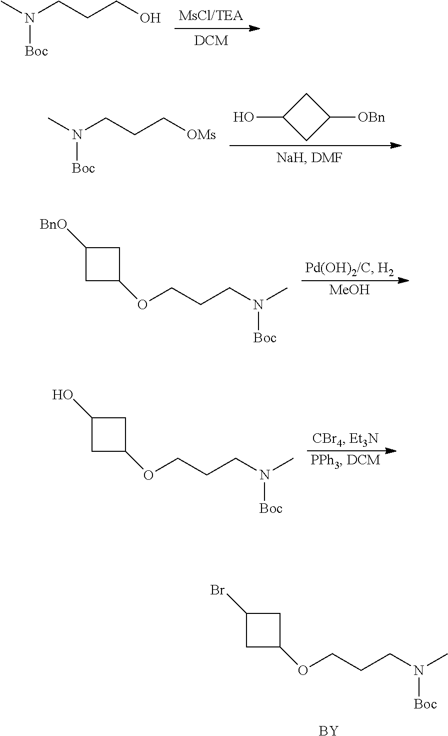

### Step 1—3-[Tert-butoxycarbonyl(methyl)amino]propyl Methanesulfonate

To a solution of tert-butyl N-(3-hydroxypropyl)-N-methyl-carbamate (11.6 g, 61.3 mmol) and Et₃N (8.06 g, 79.7 mmol, 11.09 mL) in DCM (150 mL) was added MsCl (8.12 g, 70.89 mmol, 5.49 mL) at 0° C. The mixture was stirred at 20° C. for 2 h. On completion, the reaction mixture was added to ice-water (200 mL), and then was extracted with DCM (150 mL×3). The combined organic phase was washed with brine (125 mL×3), dried with anhydrous Na₂SO₄, filtered and concentrated in vacuo to give the title compound (16.4 g) as yellow oil. ¹H NMR (400 MHz, CDCl₃) δ=4.27-4.24 (t, J=6.4 Hz, 2H), 3.37-3.34 (t, J=6.8 Hz, 2H), 3.03 (s, 3H), 2.87 (s, 3H), 2.02-1.95 (q, J=6.4 Hz, 2H), 1.46 (s, 9H).

### Step 2—Tert-butyl N-[3-(3-benzyloxycyclobutoxy)propyl]-N-methyl-carbamate

To a solution of 3-benzyloxycyclobutanol (14.2 g, 79.7 mmol) in DMF (160 mL) was added NaH (3.68 g, 92.02 mmol, 60% dispersion in mineral oil) at 0° C. slowly. The mixture was stirred at 20° C. for 0.5 h, and a solution of 3-[tert-butoxycarbonyl(methyl)amino]propylmethanesulfonate (16.4 g, 61.3 mmol) in DMF (160 mL) was added to above mixture at 20° C. The mixture was stirred at 20° C. for 15 h. On completion, the reaction mixture was added to ice-water (600 mL), and it was extracted with ethyl acetate (400 mL×3). The combined organic phase was washed with brine (150 mL×4), dried with anhydrous Na2SO4, filtered and concentrated in vacuo. The residue was purified by column chromatography (SiO₂, Petroleum ether/Ethyl acetate=100/1 to 1/5) to give the title compound (15.68 g, 73% yield) as yellow oil. ¹H NMR (400 MHz, CDCl₃) δ=7.27-7.19 (m, 5H), 4.35-4.34 (s, 2H), 3.60-3.48 (m, 2H), 3.27-3.20 (m, 4H), 2.78 (s, 3H), 2.57-2.54 (m, 2H), 1.88-1.85 (m, 2H), 1.71-1.68 (q, J=6.4 Hz, 2H), 1.38 (s, 9H).

### Step 3—Tert-butyl (3-(3-hydroxycyclobutoxy)propyl)(methyl)carbamate

To a solution of tert-butyl N-[3-(3-benzyloxycyclobutoxy)propyl]-N-methyl-carbamate (15.4 g, 44.07 mmol) in MeOH (500 mL) was added Pd/C (3.50 g, 10 wt %) and Pd(OH)₂ (3.5 g, 4.98 mmol, 20 wt % purity) under N₂ atmosphere. The suspension was degassed and purged with H₂ for three times. The mixture was then stirred under H₂ (50 psi) at 25° C. for 18 h. On completion, the mixture was filtered and concentrated in vacuo to give the title compound (11 g, 96% yield) as a colorless oil. ¹H NMR (400 MHz, CDCl₃) δ=3.93-3.91 (m, 1H), 3.52 (m, 1H), 3.35-3.28 (m, 4H), 2.85 (s, 3H), 2.73-2.70 (m, 2H), 1.89-1.75 (m, 4H), 1.46 (s, 9H).

### Step 4—Tert-butyl (3-(3-bromocyclobutoxy)propyl)(methyl)carbamate

A solution of tert-butyl N-[3-(3-hydroxycyclobutoxy)propyl]-N-methyl-carbamate (9.9 g, 38 mmol) in toluene (80 mL) was heated to 110° C. for 0.5 h. A mixture of CBr₄ (30.76 g, 92.76 mmol) in DCM (60 mL) was added to above mixture, and Et₃N (8.77 g, 86.65 mmol, 12.06 mL) was added to the above mixture. Then, a solution of PPh₃ (23.63 g, 90.09 mmol) in DCM (70 mL) was added dropwise at 5-10° C. The reaction was heated to 45° C. for 1.5 hr then stirred at 25° C. for 13 hrs. On completion, the reaction was diluted with EA (30 mL) and PE (40 mL), and filtered. The filtrate was concentrated in vacuo to give a residue. The residue was purified by column chromatography (SiO₂, Petroleum ether/Ethyl acetate=100/1 to 1/1) to give the title compound (10 g, 81% yield) as yellow oil. ¹H NMR (400 MHz, CDCl3) δ=4.53-4.48 (q, J=5.9 Hz, 1H), 4.38-4.34 (q, J=6.1 Hz, 1H), 3.35-3.27 (m, 4H), 2.85 (s, 3H), 2.66-2.63 (t, J=6.0 Hz, 4H), 1.81-1.74 (q, J=6.7 Hz, 2H), 1.46 (s, 9H).

### Example 1.79. Preparation of 3-(3-methyl-4-{3-[3-(methylamino)propoxy]cyclobutyl}-2-oxo-1,3-benzodiazol-1-yl)piperidine-2,6-dione (Intermediate BZ)

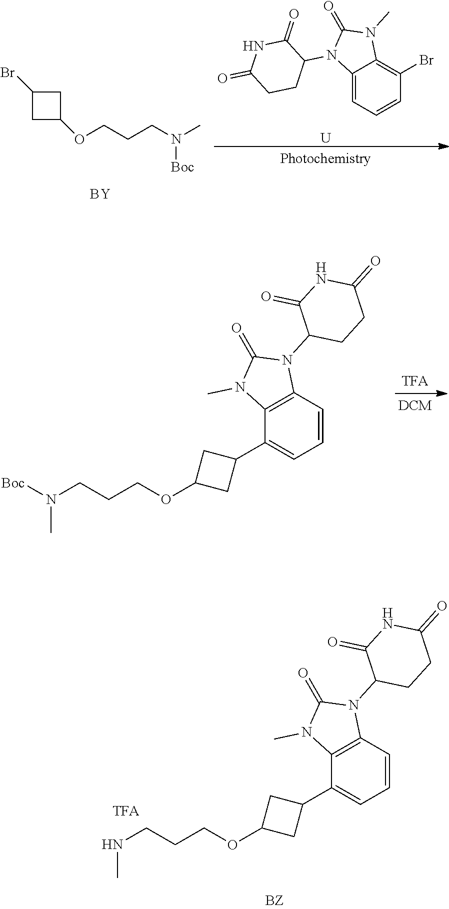

### Step 1—tert-butyl (3-(3-(1-(2,6-dioxopiperidin-3-yl)-3-methyl-2-oxo-2,3-dihydro-1H-benzo[d]imidazol-5-yl)cyclobutoxy)propyl)(methyl)carbamate

To a solution of tert-butyl N-[3-(3-bromocyclobutoxy)propyl]-N-methyl-carbamate (4.17 g, 12.9 mmol, Intermediate BY), 3-(4-bromo-3-methyl-2-oxo-benzimidazol-1-yl)piperidine-2,6-dione (3.50 g, 10.6 mmol, Intermediate U), TTMSS (2.57 g, 10.4 mmol, 3.19 mL), 4-tert-butyl-2-(4-tert-butyl-2-pyridyl)pyridine; dichloronickel (205.97 mg, 517.51 umol) and bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (116.12 mg, 103.50 umol) in DME (100 mL) was added Na₂CO₃ (2.19 g, 20.7 mmol) under N₂. The reaction was stirred and irradiated with a 34 W blue LED lamp (7 cm away), with cooling fan to keep the reaction temperature at 25° C. for 14 hr. On completion, the product was filtered to remove the insoluble material and the filter liquor was concentrated in vacuo. The residue was purified by column chromatography (SiO₂, PE:EA=3:1 to 0:1, P: Rf=0.5) to give the crude product. The product was repurified by reversed-phase HPLC (0.1% FA condition) to give the title compound (2.72 g, 52% yield) as white solid. ¹H NMR (400 MHz, CDCl₃) δ=8.15 (s, 1H), 7.17-7.08 (m, 2H), 6.71-6.69 (d, J=7.8 Hz, 1H), 5.24-5.21 (m, 1H), 4.26-4.00 (m, 2H), 3.70 (m, 3H), 3.65 (m, 1H), 3.45-3.40 (m, 2H), 3.34-3.31 (m, 2H), 2.94-2.67 (m, 7H), 2.26-2.21 (m, 1H), 1.85 (m, 2H), 1.83 (m, 2H), 1.48 (s, 9H).

### Step 2—3-(3-methyl-4-{3-[3-(methylamino)propoxy]cyclobutyl}-2-oxo-1,3-benzodiazol-1-yl)piperidine-2,6-dione Trifluoroacetate

To a stirred mixture of tert-butyl N-(3-{3-[1-(2,6-dioxopiperidin-3-yl)-3-methyl-2-oxo-1,3-benzodiazol-4-yl]cyclobutoxy}propyl)-N-methylcarbamate (100 mg, 0.200 mmol) in DCM (2 mL) was added TFA (2 mL) dropwise at rt under nitrogen atmosphere. The resulting mixture was stirred for 1 h at rt under nitrogen atmosphere. On completion, the reaction mixture was concentrated under reduced pressure. The residue was triturated with Et₂O to afford the title compound (100 mg) as a white solid. LC/MS (ESI, m/z): [(M+H)]⁺=401.2.

### Example 1.80. Preparation of 3-{3-methyl-5-[(3-methylazetidin-3-yl)methyl]-2-oxo-1,3-benzodiazol-1-yl}piperidine-2,6-dione (Intermediate CA)

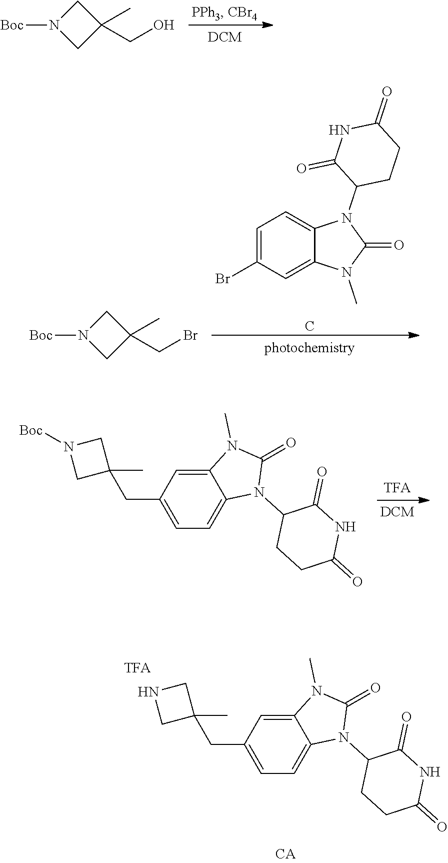

### Step 1—Tert-butyl 3-(bromomethyl)-3-methylazetidine-1-carboxylate

To a solution of tert-butyl 3-(hydroxymethyl)-3-methyl-azetidine-1-carboxylate (4 g, 20 mmol) in DCM (150 mL) was added PPh₃ (15.6 g, 59.6 mmol) and CBr₄ (19.8 g, 59.6 mmol) at 0° C. and stirred at 25° C. for 2 hr. On completion, the mixture was filtered and the filtrate was concentrated in vacuo. The residue was purified by column chromatography (SiO₂, PE:EA=20:1 to 10:1, P: Rf=0.35 (PE:EA=10:1)) to give the title compound (3.05 g, 58% yield) as colorless oil. ¹H NMR (400 MHz, CHLOROFORM-d) δ=3.75 (d, J=8.8 Hz, 2H), 3.63 (d, J=8.8 Hz, 2H), 3.53 (s, 2H), 1.45 (s, 9H), 1.40 (s, 3H).

### Step 2—Tert-butyl 3-((1-(2,6-dioxopiperidin-3-yl)-3-methyl-2-oxo-2,3-dihydro-1H-benzo[d]imidazol-5-yl)methyl)-3-methylazetidine-1-carboxylate

Tert-butyl 3-(bromomethyl)-3-methyl-azetidine-1-carboxylate (3.05 g, 11.5 mmol), 3-(5-bromo-3-methyl-2-oxo-benzimidazol-1-yl) piperidine-2,6-dione (3.00 g, 8.87 mmol, Intermediate C) Ir[dF (CF₃) ppy]2 (dtbpy) (PF₆) (99.5 mg, 88.7 μmol), NiCl₂. Dtbbpy (17.65 mg, 44.36 μmol), TTMSS (2.21 g, 8.87 mmol, 2.74 mL) and Na₂CO₃ (1.88 g, 17.7 mmol) was dissolved in DME (100 mL). The vial was sealed and placed under nitrogen. The reaction was stirred and irradiated with a 34 W blue LED lamp (7 cm away), with cooling fan to keep the reaction temperature at 25° C. for 14 hr. On completion, the mixture was filtered and the filtrate was concentrated under reduced pressure to give a residue. The crude product was purified by reversed-phase HPLC (0.1% FA) to give the title compound (2.09 g, 53% yield) as white solid. ¹H NMR (400 MHz, DMSO+D₂O) δ=6.86-6.75 (m, 2H), 6.65 (d, J=8 Hz, 1H), 5.03 (br dd, J=5.2, 12.4 Hz, 1H), 3.53 (brd, J=8.4 Hz, 2H), 3.16 (br d, J=1.2 Hz, 2H), 3.07 (s, 3H), 2.68-2.54 (m, 4H), 2.41 (br d, J=14 Hz, 2H), 1.78 (br s, 1H), 1.07 (s, 9H), 0.90 (s, 3H).

### Step 3—3-{3-methyl-5-[(3-methylazetidin-3-yl)methyl]-2-oxo-1,3-benzodiazol-1-yl}piperidine-2,6-dione Trifluoroacetate

To a stirred solution of tert-butyl 3-{[1-(2,6-dioxopiperidin-3-yl)-3-methyl-2-oxo-1,3-benzodiazol-5-yl]methyl}-3-methylazetidine-1-carboxylate (75 mg, 0.169 mmol) in DCM (6 mL) was added TFA (1.2 mL) dropwise at rt. The resulting mixture was stirred for additional 1 h at rt. On completion, the reaction mixture was concentrated under reduced pressure. The residue was triturated with Et₂O (5 mL) to afford the title compound (71 mg) as an off-white solid. LC/MS (ESI, m/z): [(M+H)]⁺=343.1.

### Example 1.81. Preparation of 3-(3-methyl-5-{4-[(methylamino)methyl]piperidin-1-yl}-2-oxo-1,3-benzodiazol-1-yl)piperidine-2,6-dione Trifluoroacetate (Intermediate CB)

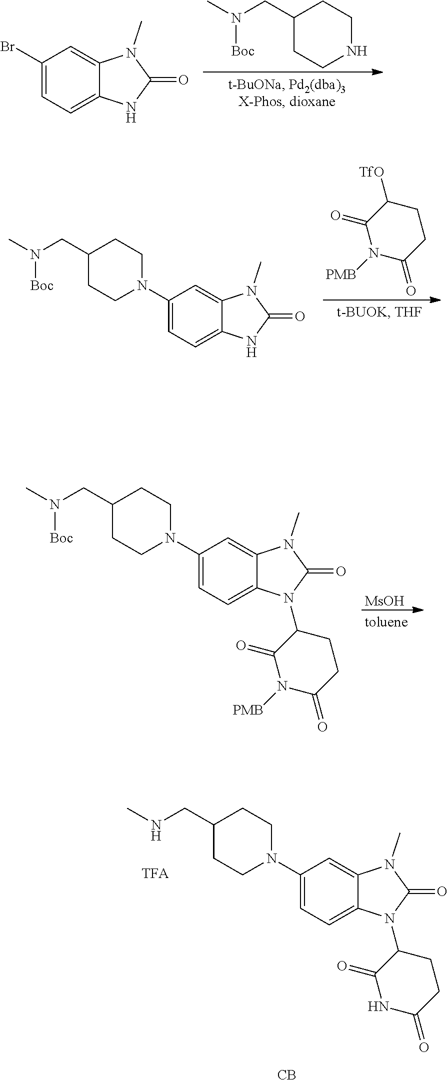

### Step 1—Tert-butyl N-methyl-N-[[1-(3-methyl-2-oxo-1H-1,3-benzodiazol-5-yl)piperidin-4-yl]methyl]carbamate

To a stirred mixture of 6-bromo-1-methyl-3H-1,3-benzodiazol-2-one (10.00 g, 44.04 mmol, CAS #305790-48-1) and tert-butyl N-methyl-N-(piperidin-4-ylmethyl)carbamate (12.07 g, 52.85 mmol, CAS #138022-04-5) in dioxane (150.00 mL) were added X-Phos (2.10 g, 4.40 mmol), Pd2(dba)3 (4.03 g, 4.40 mmol) and t-BuONa (8.46 g, 88.1 mmol) in portions at rt under nitrogen atmosphere. The resulting mixture was stirred for 16 h at 130° C. under nitrogen atmosphere. On completion, the mixture was cooled to rt and quenched with water at rt. The resulting mixture was extracted with EtOAc (3×50 mL). The combined organic layers were washed with brine (1×50 mL), and dried over anhydrous Na₂SO₄. After filtration, the filtrate was concentrated under reduced pressure. The residue was purified by silica gel column chromatography, eluted with CH₂Cl₂/EtOAc (3:1-1:3), to afford the title compound (7.3 g, 44% yield) as a pink solid. LC/MS (ESI, m/z): [(M+1)]⁺=375.2.

### Step 2—Tert-butyl N-[[1-(1-[1-[(4-methoxyphenyl)methyl]-2,6-dioxopiperidin-3-yl]-3-methyl-2-oxo-1,3-benzodiazol-5-yl)piperidin-4-yl]methyl]-N-methylcarbamate

To a stirred mixture of tert-butyl N-methyl-N-[[1-(3-methyl-2-oxo-1H-1,3-benzodiazol-5-yl)piperidin-4-yl]methyl]carbamate (7.30 g, 19.5 mmol) in THF (100.00 mL) was added t-BuOK (3.06 g, 27.3 mmol) in portions at rt. To the above mixture was added 1-[(4-methoxyphenyl)methyl]-2,6-dioxopiperidin-3-yl trifluoromethanesulfonate (8.18 g, 21.4 mmol, CAS #2304754-47-8) in portions over 5 min at rt. The resulting mixture was stirred for additional 16 h at rt. On completion, the reaction was quenched by the addition of water (100 mL) at rt. The resulting mixture was extracted with EtOAc (3×50 mL). The combined organic layers were washed with brine (2×50 mL), and dried over anhydrous Na₂SO₄. After filtration, the filtrate was concentrated under reduced pressure. The residue was purified by silica gel column chromatography, eluted with PE/EtOAc (1:1-2:1) to afford the title compound (6.6 g, 56% yield) as a green solid. LC/MS (ESI, m/z): [(M+1)]⁺=606.3.

### Step 3—3-(3-Methyl-5-[4-[(methylamino)methyl]piperidin-1-yl]-2-oxo-1,3-benzodiazol-1-yl)piperidine-2,6-dione Trifluoroacetate

To a stirred mixture of tert-butyl N-[[1-(1-[1-[(4-methoxyphenyl)methyl]-2,6-dioxopiperidin-3-yl]-3-methyl-2-oxo-1,3-benzodiazol-5-yl)piperidin-4-yl]methyl]-N-methylcarbamate (5.00 g, 8.25 mmol) in toluene (50.00 mL) was added MsOH (20.00 mL) dropwise at rt. The resulting mixture was stirred for 4 h at 110° C. under nitrogen atmosphere. On completion, the reaction was quenched with water at rt. The resulting mixture was concentrated under reduced pressure. The residue was purified by reverse phase flash chromatography (Column: WelFlash™ C18-I, 20-40 ?m, 330 g; Eluent A: Water (plus 10 mmol/L TFA); Eluent B: ACN; Gradient: 2%-10% B in 25 min; Flow rate: 80 mL/min; Detector: 220/254 nm; desired fractions were collected at 3% B) and concentrated under reduced pressure to afford the title compound (1.2 g, 30% yield) as a white solid. LC/MS (ESI, m/z): [(M+1)]⁺=386.2.

### Example 1.82. Preparation of 3-[3-methyl-4-({4-[(methylamino)methyl]piperidin-1-yl}methyl)-2-oxo-1,3-benzodiazol-1-yl]piperidine-2,6-dione (Intermediate CC)

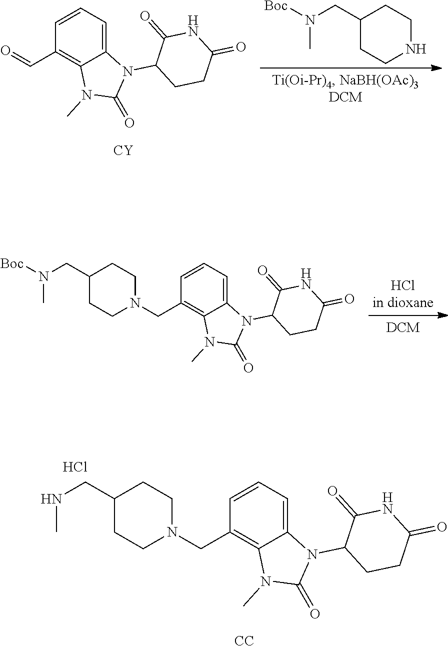

### Step 1—Tert-butyl N-[(1-[[1-(2,6-dioxopiperidin-3-yl)-3-methyl-2-oxo-1,3-benzodiazol-4-yl]methyl]piperidin-4-yl)methyl]-N-methylcarbamate

To a stirred mixture of 1-(2,6-dioxopiperidin-3-yl)-3-methyl-2-oxo-1,3-benzodiazole-4-carbaldehyde (10.00 g, 34.81 mmol, Intermediate CY) and tert-butyl N-methyl-N-(piperidin-4-ylmethyl)carbamate (9.54 g, 41.8 mmol, CAS #138022-04-5) in DCM (200.00 mL) was added Ti(Oi-Pr)₄ (29.68 g, 104.4 mmol) dropwise at rt. The resulting mixture was stirred for 10 min at rt under air atmosphere. To the above mixture was added NaBH(OAc)₃ (14.76 g, 69.62 mmol) in portions over 10 min at rt. The resulting mixture was stirred for additional 16 h at rt. On completion, the reaction was quenched by the addition of water (50 mL) at rt. The resulting mixture was filtered and the filter cake was washed with methanol (3×50 mL). The filtrate was concentrated under reduced pressure and the resulting mixture was extracted with CH₂Cl₂ (2×100 mL). The combined organic layers were washed with brine (2×50 mL), and dried over anhydrous Na₂SO₄. After filtration, the filtrate was concentrated under reduced pressure. The residue was purified by trituration with ethyl ether (20 mL) to give the title compound (7.5 g, 43% yield) as a brown solid. LC/MS (ESI, m/z): [(M+1)]⁺=500.3.

### Step 2—3-[3-Methyl-4-([4-[(methylamino)methyl]piperidin-1-yl]methyl)-2-oxo-1,3-benzodiazol-1-yl]piperidine-2,6-dione Hydrochloride

To a stirred solution of tert-butyl N-[(1-[[1-(2,6-dioxopiperidin-3-yl)-3-methyl-2-oxo-1,3-benzodiazol-4-yl]methyl]piperidin-4-yl)methyl]-N-methylcarbamate (7.50 g, 15.0 mmol) in dioxane (20.00 mL) was added 4 M HCl (gas) in 1,4-dioxane (20.00 mL) dropwise at rt. The resulting mixture was stirred for 2 h at rt under air atmosphere. On completion, the precipitated solids were collected by filtration and washed with ethyl ether (3×20 mL) to give the title compound (6.3 g, 96% yield) as a brown solid. LC/MS (ESI, m/z): [(M+1)]⁺=400.3

### Example 1.83. Preparation of N-[(1R,2S)-2-fluorocyclopropyl]-6-({5′-formyl-2-oxo-[1,2′-bipyridin]-3-yl}amino)-8-(methylamino)imidazo[1,2-b]pyridazine-3-carboxamide (Intermediate CD)

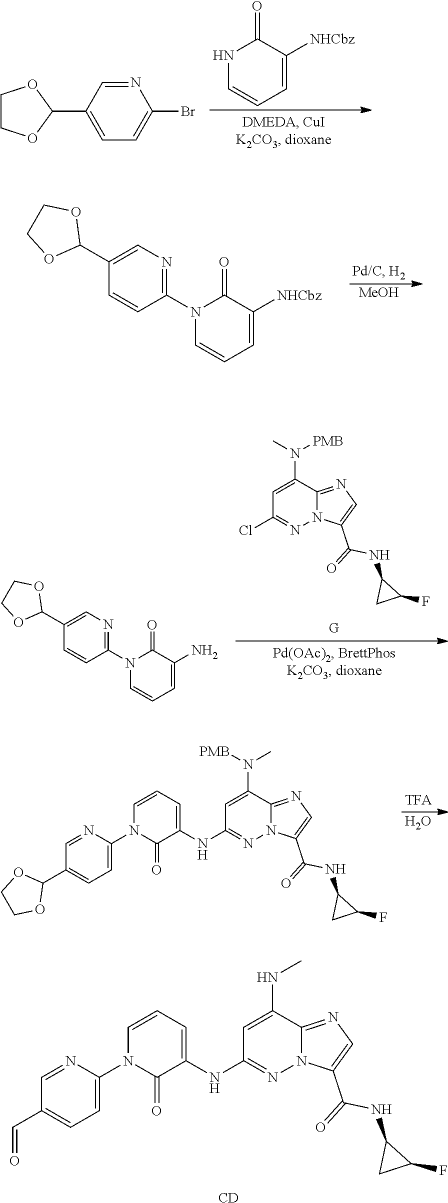

### Step 1—Benzyl N-[5′-(1,3-dioxolan-2-yl)-2-oxo-[1,2′-bipyridin]-3-yl]carbamate

To a stirred mixture of 2-bromo-5-(1,3-dioxolan-2-yl)pyridine (1.4 g, 6.1 mmol, synthesized via Step 1 of Intermediate BC) and benzyl N-(2-oxo-1H-pyridin-3-yl)carbamate (1.63 g, 6.69 mmol, CAS #147269-67-8) in dioxane (20 mL) were added DMEDA (107.29 mL, 1.217 mmol), CuI (0.23 g, 1.2 mmol) and K₂CO₃ (1.68 g, 12.2 mmol) in turns at rt under nitrogen atmosphere. The resulting mixture was stirred for 2 h at 100° C. under nitrogen atmosphere. On completion, the mixture was cooled to rt and concentrated under reduced pressure. The resulting mixture was extracted with EtOAc (3×20 mL). The combined organic layers were washed with brine (3×20 mL), and dried over anhydrous Na₂SO₄. After filtration, the filtrate was concentrated under reduced pressure. The residue was purified by silica gel column chromatography, eluted with PE/EA (1:1), to afford the title compound (1.00 g, 42% yield) as a yellow oil. LC/MS (ESI, m/z): [(M+H)]⁺=394.1.

### Step 2—3-Amino-5′-(1,3-dioxolan-2-yl)-[1,2′-bipyridin]-2-one

To a stirred mixture of benzyl N-[5′-(1,3-dioxolan-2-yl)-2-oxo-[1,2′-bipyridin]-3-yl]carbamate (3 g, 8 mmol) in MeOH (20 mL) was added 10 wt % Pd/C (0.30 g) at rt under nitrogen atmosphere. The reaction system was degassed under vacuum and purged with H₂ several times, then it was hydrogenated under H₂ balloon (1 atm) at rt for 1 h. After completion of the reaction, Pd/C was filtered off through celite and the corresponding filtrate was concentrated under reduced pressure to afford the title compound (1.90 g, 96% yield) as a yellow oil. LC/MS (ESI, m/z): [(M+H)]⁺=260.1.

### Step 3—6-{[5′-(1,3-dioxolan-2-yl)-2-oxo-[1,2′-bipyridin]-3-yl]amino}-N-[(1R,2S)-2-fluorocyclopropyl]-8-{[(4-methoxyphenyl)methyl](methyl)amino}imidazo[1,2-b]pyridazine-3-carboxamide

To a stirred mixture of 6-chloro-N-[(1R,2S)-2-fluorocyclopropyl]-8-{[(4-methoxyphenyl)methyl](methyl)amino}imidazo[1,2-b]pyridazine-3-carboxamide (2.6 g, 6.4 mmol, Intermediate G) and 3-amino-5′-(1,3-dioxolan-2-yl)-[1,2′-bipyridin]-2-one (2 g, 8 mmol) in dioxane (50 mL) were added Pd(OAc)₂ (0.14 g, 0.64 mmol), BrettPhos (0.35 g, 0.64 mmol) and K₂CO₃ (1.78 g, 12.9 mmol) in turns at rt under nitrogen atmosphere. The resulting mixture was stirred for 16 h at 100° C. under nitrogen atmosphere. On completion, the reaction mixture was cooled to rt and filtered, then the filter cake was washed with dioxane (2×10 mL). The filtrate was concentrated under reduced pressure. The crude product was purified by reverse phase flash (Column: WelFlash™ C18-I, 20-40 um, 330 g; Eluent A: Water (plus 10 mmol/L FA); Eluent B: ACN; Gradient: 25%-55% B in 35 min; Flow rate: 80 mL/min; Detector: 254 nm; desired fractions were collected at 40% B) and concentrated under reduced pressure to afford the title compound (3.9 g, 97% yield) as a yellow solid. LC/MS (ESI, m/z): [(M+H)]⁺=627.2.

### Step 4—N-[(1R,2S)-2-fluorocyclopropyl]-6-({5′-formyl-2-oxo-[1,2′-bipyridin]-3-yl}amino)-8-(methylamino)imidazo[1,2-b]pyridazine-3-carboxamide

To a stirred mixture of 6-{[5′-(1,3-dioxolan-2-yl)-2-oxo-[1,2′-bipyridin]-3-yl]amino}-N-[(1R,2S)-2-fluorocyclopropyl]-8-{[(4-methoxyphenyl)methyl](methyl)amino}imidazo[1,2-b]pyridazine-3-carboxamide (3.9 g, 6.2 mmol) in H₂O (20 mL) and TFA (20 mL) at rt under nitrogen atmosphere. The resulting mixture was stirred for 16 h at 50° C. under nitrogen atmosphere. On completion, the reaction was cooled to rt and basified to pH 7 with saturated NaHCO₃ (aq.). The precipitated solids were collected by filtration and washed with water (3×10 mL). The crude product was purified by reverse phase flash chromatography (Column: WelFlash™ C18-I, 20-40 um, 330 g; Eluent A: Water (10 mmol/L NH₄HCO₃); Eluent B: ACN; Gradient: 25%-55% B in 35 min; Flow rate: 80 mL/min; Detector: 254 nm; desired fractions were collected at 40% B) and concentrated under reduced pressure to afford the title compound (1.1 g, 38% yield) as a yellow solid. ¹H NMR (400 MHz, DMSO-d₆) δ 10.19 (s, 1H), 9.17-9.14 (m, 1H), 8.70 (s, 1H), 8.64 (d, J=4.2 Hz, 1H), 8.47 (dd, J=8.4, 2.3 Hz, 1H), 8.14 (d, J=8.4 Hz, 1H), 8.02 (d, J=7.3 Hz, 1H), 7.92 (s, 1H), 7.65 (dd, J=7.1, 1.7 Hz, 1H), 7.55-7.47 (m, 1H), 6.46 (t, J=7.2 Hz, 1H), 6.41 (s, 1H), 5.00-4.74 (m, 1H), 3.04-2.96 (m, 1H), 2.88 (d, J=4.9 Hz, 3H), 1.30-1.18 (m, 1H), 1.05-0.92 (m, 1H). LC/MS (ESI, m/z): [(M+H)]⁺=463.1.

### Example 1.84. Preparation of 3-{3-methyl-2-oxo-5-[1-(piperidin-4-ylmethyl)piperidin-4-yl]-1,3-benzodiazol-1-yl}piperidine-2,6-dione (Intermediate CE)

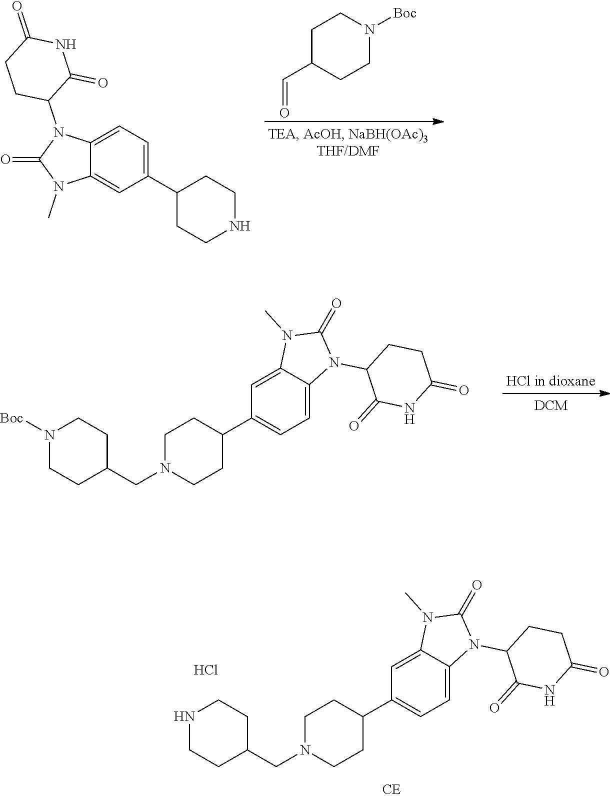 [SMILES: Cn1c(=O)n(C2CCC(=O)NC2=O)c2ccc(C3CCNCC3)cc21 ; CC(C)(C)OC(=O)N1CCC(C=O)CC1 ; Cn1c(=O)n(C2CCC(=O)NC2=O)c2ccc(C3CCN(CC4CCN(C(=O)OC(C)(C)C)CC4)CC3)cc21 ; Cn1c(=O)n(C2CCC(=O)NC2=O)c2ccc(C3CCN(CC4CCNCC4)CC3)cc21 ; Cl]

### Step 1—Tert-butyl 4-((4-(1-(2,6-dioxopiperidin-3-yl)-3-methyl-2-oxo-2,3-dihydro-1H-benzo[d]imidazol-5-yl)piperidin-1-yl)methyl)piperidine-1-carboxylate

To a solution of 3-[3-methyl-2-oxo-5-(4-piperidyl)benzimidazol-1-yl]piperidine-2,6-dione (2.89 g, 6.33 mmol, CAS #2502189-47-9) in tetrahydrofuran (30 mL) and dimethylformamide (10 mL) was added triethylamine (640 mg, 6.33 mmol) at 25° C. and the mixture was stirred for 0.5 hour. Then tert-butyl 4-formylpiperidine-1-carboxylate (1.35 g, 6.33 mmol) and acetic acid (1.90 g, 31.6 mmol) was added at 25° C. and the mixture was stirred for 0.5 hour. Then sodium triacethoxy borohydride (2.68 g, 12.6 mmol) was added at 25° C. The mixture was stirred at 25° C. for 11 hours. On completion, the reaction mixture was partitioned between water (100 mL) and ethyl acetate (400 mL). The organic phase was separated, washed with brine (150 mL×2), dried over with anhydrous sodium sulfate, filtered and concentrated under reduced pressure to give a residue. The residue was washed with ethyl acetate 10 mL and petroleum ether (10 mL) to give the title compound (2.71 g, 79% yield) as a white solid. ¹H NMR (400 MHz, DMSO-d₆) δ=11.09 (s, 1H), 7.09 (s, 1H), 7.02 (d, J=8.0 Hz, 1H), 6.92 (d, J=8.4 Hz, 1H), 5.34 (dd, J=5.2, 12.8 Hz, 1H), 3.93 (d, J=10.4 Hz, 2H), 3.42-3.37 (m, 2H), 3.34-3.33 (m, 3H), 3.06-2.84 (m, 3H), 2.78-2.57 (m, 5H), 2.24-2.13 (m, 1H), 2.06-1.96 (m, 2H), 1.86-1.63 (m, 7H), 1.40 (s, 9H), 1.09-0.86 (m, 2H); LC-MS (ESI+) m/z 540.2 (M+H)⁺.

### Step 2—3-{3-Methyl-2-oxo-5-[1-(piperidin-4-ylmethyl)piperidin-4-yl]-1,3-benzodiazol-1-yl}piperidine-2,6-dione Hydrochloride

To a stirred mixture of tert-butyl 4-({4-[1-(2,6-dioxopiperidin-3-yl)-3-methyl-2-oxo-1,3-benzodiazol-5-yl]piperidin-1-yl}methyl)piperidine-1-carboxylate (75 mg, 0.139 mmol) in DCM (2 mL) was added 4 M HCl (gas) in 1,4-dioxane (8.82 mL) dropwise at rt under nitrogen atmosphere. The resulting mixture was stirred for 1 h at rt under nitrogen atmosphere. On completion, the reaction mixture was concentrated under reduced pressure. The residue was triturated with Et₂O to afford the title compound (70 mg) as a white solid. LC/MS (ESI, m/z): [(M+H)]⁺=440.2.

### Example 1.85. Preparation of 3-[5-(3-{2,6-Diazaspiro[3.4]octan-2-yl}prop-1-yn-1-yl)-3-methyl-2-oxo-1,3-benzodiazol-1-yl]piperidine-2,6-dione (Intermediate CF)

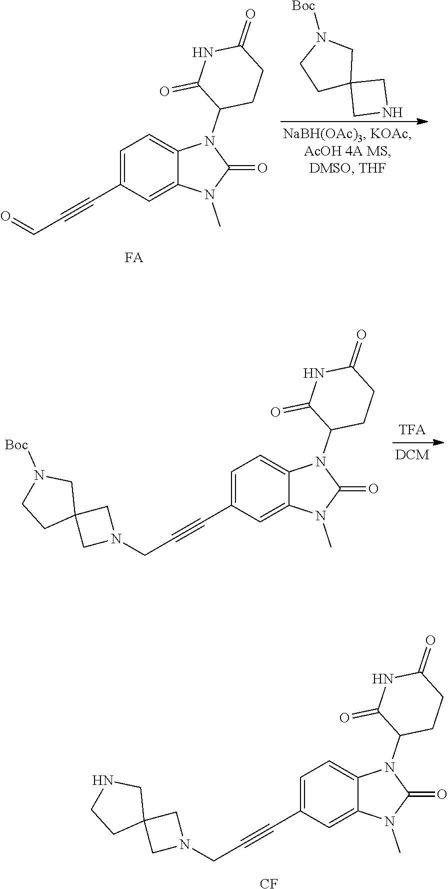

### Step 1—Tert-butyl 2-(3-(1-(2,6-dioxopiperidin-3-yl)-3-methyl-2-oxo-2,3-dihydro-1H-benzo[d]imidazol-5-yl)prop-2-yn-1-yl)-2,6-diazaspiro[3.4]octane-6-carboxylate

To a solution of 3-(1-(2,6-dioxopiperidin-3-yl)-3-methyl-2-oxo-2,3-dihydro-1H-benzo[d]imidazol-5-yl)propiolaldehyde (2.50 g, 6.42 mmol, Intermediate FA) in DMSO (20 mL) and THF (20 mL) was added KOAc (1.89 g, 19.3 mmol), AcOH (1.16 g, 19.3 mmol, 1.10 mL), 4 Å molecular sieves (3 g), and tert-butyl 2,6-diazaspiro[3.4]octane-6-carboxylate (1.60 g, 6.42 mmol, CAS #885270-84-8, HCl salt) at 0° C. and the mixture was stirred at 0° C. for 0.5 h. Then NaBH(OAc)₃ (4.09 g, 19.3 mmol) was added to at 0° C. and the reaction mixture was stirred at 25° C. for 1 h. On completion, the mixture was filtered and the filtrate was concentrated. The residue was purified by reversed-phase HPLC (ACN/0.1% FA=25%) to afford the title compound (1.60 g, 46% yield) as a yellow solid. ¹H NMR (400 MHz, DMSO-d₆) δ=11.12 (br d, J=1.2 Hz, 1H), 8.19 (s, 1H), 7.31 (s, 1H), 7.15 (br d, J=5.2 Hz, 2H), 5.39 (br dd, J=5.2, 13.2 Hz, 1H), 3.46 (s, 2H), 3.21 (br s, 6H), 2.90-2.85 (m, 1H), 2.72 (br s, 1H), 2.63 (br d, J=19.6 Hz, 2H), 2.07-1.92 (m, 4H), 1.39 (s, 9H).

### Step 2—3-(5-(3-(2,6-Diazaspiro[3.4]octan-2-yl)prop-1-yn-1-yl)-3-methyl-2-oxo-2,3-dihydro-1H-benzo[d]imidazol-1-yl)piperidine-2,6-dione

To a solution of tert-butyl 2-(3-(1-(2,6-dioxopiperidin-3-yl)-3-methyl-2-oxo-2,3-dihydro-1H-benzo[d]imidazol-5-yl)prop-2-yn-1-yl)-2,6-diazaspiro[3.4]octane-6-carboxylate (1.60 g, 3.15 mmol) in DCM (15 mL) was added TFA (4.62 g, 40.5 mmol) and the reaction mixture was stirred at 25° C. for 1 h. On completion, the reaction mixture was diluted with water (20 mL), and extracted with EtOAc (20 mL×3). The aqueous phase was lyophilized to afford the title compound (1.64 g, 95% yield, TFA salt) as a yellow solid. LC-MS (ESI⁺) m/z 407.9 (M+H)⁺. ¹H NMR (400 MHz, DMSO-d₆) δ=11.13 (s, 1H), 9.07-8.93 (m, 2H), 7.35 (s, 1H), 7.25-7.18 (m, 2H), 5.41 (dd, J=5.2, 13.2 Hz, 1H), 4.38 (s, 2H), 4.26-4.16 (m, 4H), 3.36 (s, 3H), 2.92-2.85 (m, 1H), 2.73-2.61 (m, 3H), 2.31-2.13 (m, 3H), 2.06-2.01 (m, 1H).

### Example 1.86. Preparation of 3-(3-Methyl-2-oxo-5-piperazin-1-yl-benzimidazol-1-yl)piperidine-2,6-dione (Intermediate CG)

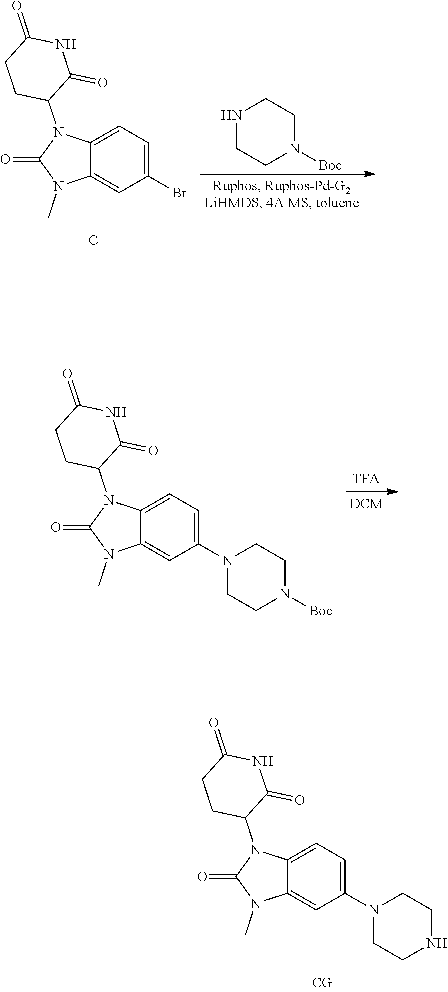

### Step 1—Tert-butyl 4-[1-(2,6-dioxo-3-piperidyl)-3-methyl-2-oxo-benzimidazol-5-yl]piperazine-1-carboxylate

To a solution of 3-(5-bromo-3-methyl-2-oxo-benzimidazol-1-yl)piperidine-2,6-dione (8.00 g, 23.6 mmol, Intermediate C), tert-butylpiperazine-1-carboxylate (6.61 g, 35.5 mmol), [2-(2-aminophenyl)phenyl]-chloro-palladium; dicyclohexyl-[2-(2,6-diisopropoxyphenyl)phenyl]phosphane (3.68 g, 4.73 mmol), dicyclohexyl(2′,6′-diisopropoxy-[1,1′-biphenyl]-2-yl)phosphane (2.21 g, 4.73 mmol) and 4 Å molecular sieves (1 g) in toluene (160 mL) was added LiHMDS (1 M, 142 mL) in one portion. The reaction was degassed with nitrogen three times, then stirred at 25° C. for 30 mins, then the mixture was heated at 80° C. under nitrogen for 2 hrs. On completion, the reaction mixture was quenched by addition of 1M hydrochloric acid (300 mL) at 0° C., and then extracted with EA (2×300 mL). The water layers were added saturated sodium bicarbonate solution to pH=7-8, then then extracted with ethyl acetate (300 mL×2). Then organic phase was washed with brine (200 ml), dried over anhydrous sodium sulfate, filtered and concentrated under reduced pressure to give the title compound (2.50 g, 16% yield) as yellow solid. LC-MS (ESI+) m/z 443.9 (M+H)⁺.

### Step 2—3-(3-Methyl-2-oxo-5-piperazin-1-yl-benzimidazol-1-yl)piperidine-2,6-dione

To a solution of tert-butyl 4-[1-(2,6-dioxo-3-piperidyl)-3-methyl-2-oxo-benzimidazol-5-yl]piperazine-1-carboxylate (2.30 g, 5.19 mmol) in dichloromethane (40 mL) was added trifluoroacetic acid (15.4 g, 135 mmol). The mixture was stirred at 25° C. for 0.5 hour. On completion, the mixture was concentrated in vacuo to give the title compound (1.80 g) as yellow oil. LC-MS (ESI+) m/z 344.1 (M+H)⁺.

### Example 1.87. Preparation of 3-[4-(8-aminooctyl)-3-methyl-2-oxo-1,3-benzodiazol-1-yl]piperidine-2,6-dione (Intermediate CI)

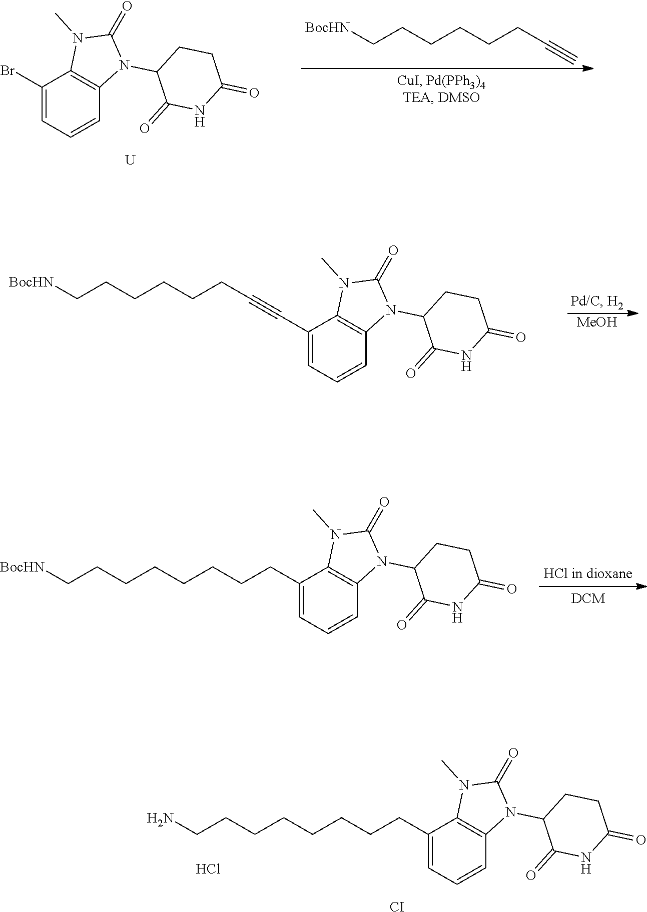

### Step 1—Tert-butyl N-[8-[1-(2,6-dioxopiperidin-3-yl)-3-methyl-2-oxo-1,3-benzodiazol-4-yl]oct-7-yn-1-yl]carbamate

To a stirred mixture of 3-(4-bromo-3-methyl-2-oxo-2,3-dihydro-1H-benzo[d]imidazol-1-yl)piperidine-2,6-dione (8.00 g, 23.7 mmol, Intermediate U) and tert-butyl N-(oct-7-yn-1-yl)carbamate (10.66 g, 47.315 mmol, CAS #1451262-84-2) in DMA (50.00 mL) and TEA (25.00 mL) were added Pd(PPh₃)₄ (2.73 g, 2.366 mmol) and CuI (450.55 mg, 2.366 mmol) in portions at rt under nitrogen atmosphere. The resulting mixture was stirred for 3 h at 80° C. under nitrogen atmosphere. The resulting mixture was diluted with water (100 mL). The resulting mixture was extracted with EtOAc (3×75 mL). The combined organic layers were washed with brine (3×50 mL), dried over anhydrous Na₂SO₄. After filtration, the filtrate was concentrated under reduced pressure. The residue product was purified by reverse phase flash (Column: Spherical C18, 20-40 um, 330 g; Mobile Phase A: Water (0.05% FA), Mobile Phase B: ACN; Flow rate: 45 mL/min; Gradient (B %): 5%˜5%, 8 min; 30%˜60%, 30 min; 60%˜95%; 0 min; 95%, 5 min; Detector: 254 nm; Rt: 23.4 min.) to afford the title compound (6 g, 53% yield) as a light yellow solid. LC/MS (ESI, m/z): [(M+1)]⁺=483.2.

### Step 2—Tert-butyl N-[8-[1-(2,6-dioxopiperidin-3-yl)-3-methyl-2-oxo-1,3-benzodiazol-4-yl]octyl]carbamate

To a stirred solution of tert-butyl N-[8-[1-(2,6-dioxopiperidin-3-yl)-3-methyl-2-oxo-1,3-benzodiazol-4-yl]oct-7-yn-1-yl]carbamate (5.50 g, 11.4 mmol) in MeOH (500.00 mL) was added Pd/C (1.82 g, 17.096 mmol, 10 wt %) at rt under nitrogen atmosphere. The reaction system was degassed under vacuum and purged with H₂ several times, then it was hydrogenated under H₂ balloon (1 atm) at rt for 16 h. After completion of the reaction, Pd/C was filtered off through celite. The filter cake was washed with MeOH (3×100 mL). The corresponding filtrate was concentrated under reduced pressure to afford the title compound (5 g, 90% yield) as a white solid. LC/MS (ESI, m/z): [(M+1)]⁺=487.2.

### Step 3—3-[4-(8-Aminooctyl)-3-methyl-2-oxo-1,3-benzodiazol-1-yl]piperidine-2,6-dione Hydrochloride

To a stirred solution/mixture of tert-butyl N-[8-[1-(2,6-dioxopiperidin-3-yl)-3-methyl-2-oxo-1,3-benzodiazol-4-yl]octyl]carbamate (3.28 g, 6.740 mmol) in DCM (50.00 mL) was 4 M HCl (gas) in 1,4-dioxane (10.00 mL) dropwise at rt under nitrogen atmosphere. The resulting mixture was stirred for 16 h at rt under nitrogen atmosphere. On completion, the reaction mixture was concentrated under reduced pressure to afford the title compound (2.52 g, 97% yield) as a white solid. LC/MS (ESI, m/z): [(M+1)]⁺=387.2.

### Example 1.88. Preparation of methyl 3-(3-amino-2-oxopyridin-1-yl)benzoate (Intermediate CJ)

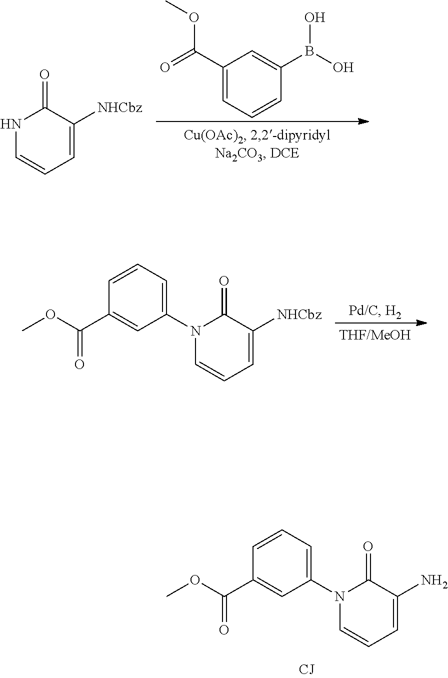 [SMILES: O=C(Nc1ccc[nH]c1=O)OCc1ccccc1 ; COC(=O)c1cccc(B(O)O)c1 ; COC(=O)c1cccc(-n2cccc(NC(=O)OCc3ccccc3)c2=O)c1 ; COC(=O)c1cccc(-n2cccc(N)c2=O)c1]

### Step 1—Methyl 3-(3-{[(benzyloxy)carbonyl]amino}-2-oxopyridin-1-yl)benzoate

To a stirred solution of benzyl N-(2-oxo-1H-pyridin-3-yl)carbamate (12.5 g, 51.2 mmol) and methyl 3-bromobenzoate (13.21 g, 61.41 mmol) in DCE (200 mL) were added Cu(OAc)₂ (11.15 g, 61.41 mmol) and Na₂CO₃ (10.85 g, 102.4 mmol) in turns at rt under air atmosphere. The resulting mixture was stirred for 16 h at 70° C. under oxygen atmosphere. On completion, the reaction mixture was filtered, the filter cake was washed with EtOAc (3×30 mL). The filtrate was concentrated under reduced pressure. The residue was diluted with water (200 mL) and was extracted with EtOAc (3×50 mL). The combined organic layers were washed with brine (3×20 mL), and dried over anhydrous Na₂SO₄. After filtration, the filtrate was concentrated under reduced pressure. The residue was purified by silica gel column chromatography, eluted with PE/EA (1:1) to afford the title compound (3 g, 16% yield) as a white solid. LC/MS (ESI, m/z): [(M+H)]⁺=379.2.

### Step 2—Methyl 3-(3-amino-2-oxopyridin-1-yl)benzoate

To a stirred solution of methyl 3-(3-{[(benzyloxy)carbonyl]amino}-2-oxopyridin-1-yl)benzoate (3 g, 8 mmol) in THF (25 mL) and MeOH (25 mL) was added 10 wt % Pd/C (0.5 g) under nitrogen atmosphere. The reaction system was degassed under vacuum and purged with H₂ several times, then it was hydrogenated under H₂ balloon (1 atm) at 25° C. for 2 h. After completion of the reaction, Pd/C was filtered through celite and the corresponding filtrate was concentrated under reduced pressure to afford the title compound (1.9 g, 98% yield) as a white solid. LC/MS (ESI, m/z): [(M+H)]⁺=245.1.

### Example 1.89. Preparation of 3-{[(1R,2S)-2-fluorocyclopropyl]carbamoyl}-6-({2-oxo-[1,2′-bipyridin]-3-yl}amino)imidazo[1,2-b]pyridazin-8-yl trifluoromethanesulfonate (Intermediate CK)

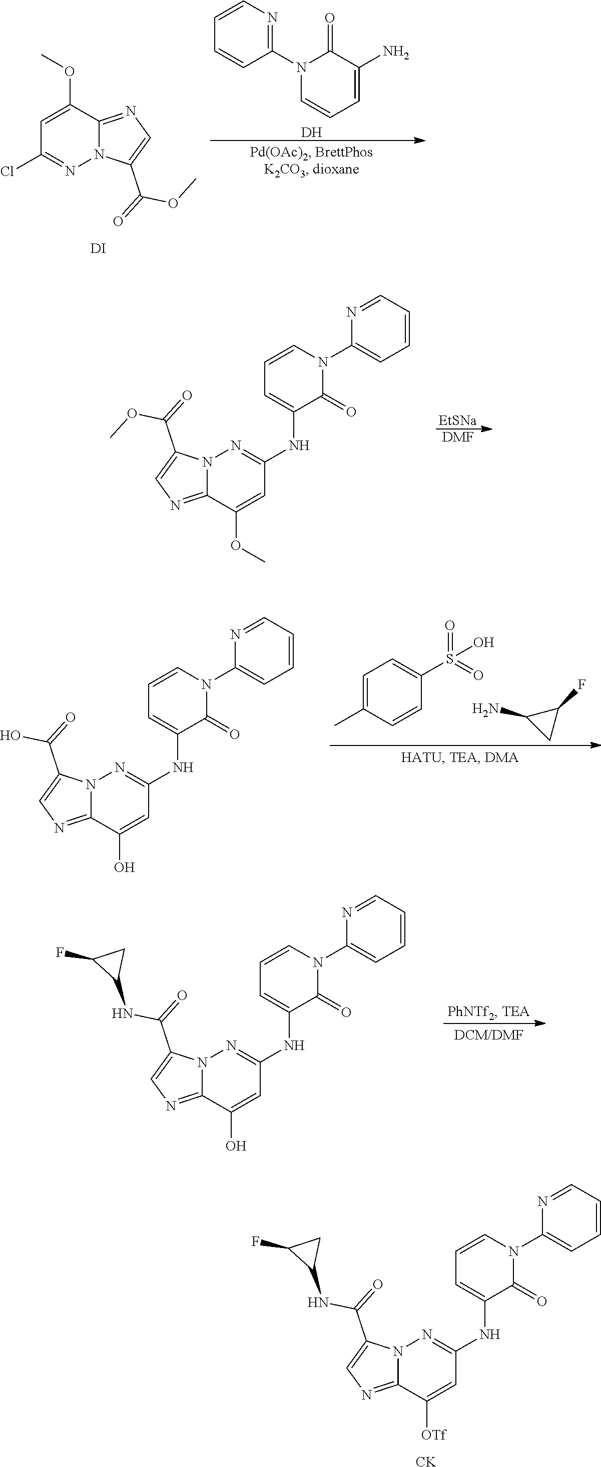

### Step 1—Methyl 8-methoxy-6-({2-oxo-[1,2′-bipyridin]-3-yl}amino)imidazo[1,2-b]pyridazine-3-carboxylate

To a stirred solution of methyl 6-chloro-8-methoxyimidazo[1,2-b]pyridazine-3-carboxylate (4.2 g, 17 mmol, Intermediate DI) and 3-amino-[1,2′-bipyridin]-2-one (3.25 g, 17.4 mmol, Intermediate DH) in DMA (50 mL) were added BrettPhos (0.93 g, 1.7 mmol), Pd(OAc)₂ (0.39 g, 1.7 mmol) and K₂CO₃ (4.80 g, 34.8 mmol) in portions at rt. The resulting mixture was stirred for additional 16 h at 90° C. The residue was purified by reverse phase flash chromatography (Column: WelFlash™ C18-I, 20-40 um, 330 g; Eluent A: Water (plus 10 mmol/L FA); Eluent B: ACN; Gradient: 95-95% B in 25 min; Flow rate: 80 mL/min; Detector: 220/254 nm; desired fractions were collected at 95% B) and concentrated under reduced pressure to afford the title compound (1.7 g, 25% yield) as a brown solid. LC/MS (ESI, m/z): [(M+H)]⁺=393.1.

### Step 2—8-Hydroxy-6-({2-oxo-[1,2′-bipyridin]-3-yl}amino)imidazo[1,2-b]pyridazine-3-carboxylic acid

To a mixture of methyl 8-methoxy-6-({2-oxo-[1,2′-bipyridin]-3-yl}amino)imidazo[1,2-b]pyridazine-3-carboxylate (5.9 g, 15 mmol) in DMF (50 mL) was added (ethylsulfanyl)sodium (12.67 g, 150.7 mmol) dropwise at rt. The reaction mixture was stirred for 2 h at 110° C. under nitrogen atmosphere. On completion, the reaction mixture was triturated with ethyl ether. The precipitated solids were collected by filtration and washed with DCM. The resulting solid was dried under vacuum to afford the title compound (6 g) as a brown solid. LC/MS (ESI, m/z): [(M+H)]⁺=365.1.

### Step 3—N-[(1R,2S)-2-fluorocyclopropyl]-8-hydroxy-6-({2-oxo-[1,2′-bipyridin]-3-yl}amino)imidazo[1,2-b]pyridazine-3-carboxamide

To a stirred solution of 8-hydroxy-6-({2-oxo-[1,2′-bipyridin]-3-yl}amino)imidazo[1,2-b]pyridazine-3-carboxylic acid (2 g, 5 mmol) and (1R,2S)-2-fluorocyclopropan-1-amine; para-toluene sulfonate (2.04 g, 8.24 mmol) in DMA (25 mL) were added HATU (3.13 g, 8.24 mmol) and TEA (2.28 mL, 16.47 mmol) in turns at rt under air atmosphere. The resulting mixture was stirred for 3 h at rt. On completion, the reaction mixture was purified by reverse phase flash chromatography (Column: WelFlash™ C18-I, 20-40 um, 330 g; Eluent A: Water (plus 10 mmol/L FA); Eluent B: ACN; Gradient: 25%-31% B in 25 min; Flow rate: 80 mL/min; Detector: 220/254 nm; desired fractions were collected at 30% B) and concentrated under reduced pressure to afford the title compound (200 mg, 9% yield) as a brown solid. LC/MS (ESI, m/z): [(M+H)]⁺=422.1.

### Step 4—3-{[(1R,2S)-2-fluorocyclopropyl]carbamoyl}-6-({2-oxo-[1,2′-bipyridin]-3-yl}amino)imidazo[1,2-b]pyridazin-8-yl trifluoromethanesulfonate

To a stirred solution of N-[(1R,2S)-2-fluorocyclopropyl]-8-hydroxy-6-({2-oxo-[1,2′-bipyridin]-3-yl}amino)imidazo[1,2-b]pyridazine-3-carboxamide (200 mg, 0.5 mmol) and 1,1,1-trifluoro-N-phenyl-N-(trifluoromethane)sulfonylmethanesulfonamide (593.45 mg, 1.662 mmol) in DCM (5 mL) was added TEA (0.13 mL, 0.95 mmol) at rt under nitrogen atmosphere. The resulting mixture was stirred for 2 h at rt under nitrogen atmosphere. On completion, the reaction mixture was concentrated under reduced pressure. The residue was purified by reverse flash chromatography (column, silica gel; mobile phase, ACN in water, 35% to 55% gradient in 35 min; detector, UV 254 nm; the fractions was collected at 50%) and concentrated under reduced pressure to afford the title compound (50 mg, 19% yield) as a yellow green solid. LC/MS (ESI, m/z): [(M+H)]⁺=554.1.

### Example 1.90. Preparation of 3-(5-(8-aminooctyl)-3-methyl-2-oxo-2,3-dihydro-1H-benzo[d]imidazol-1-yl)piperidine-2,6-dione (Intermediate CL)

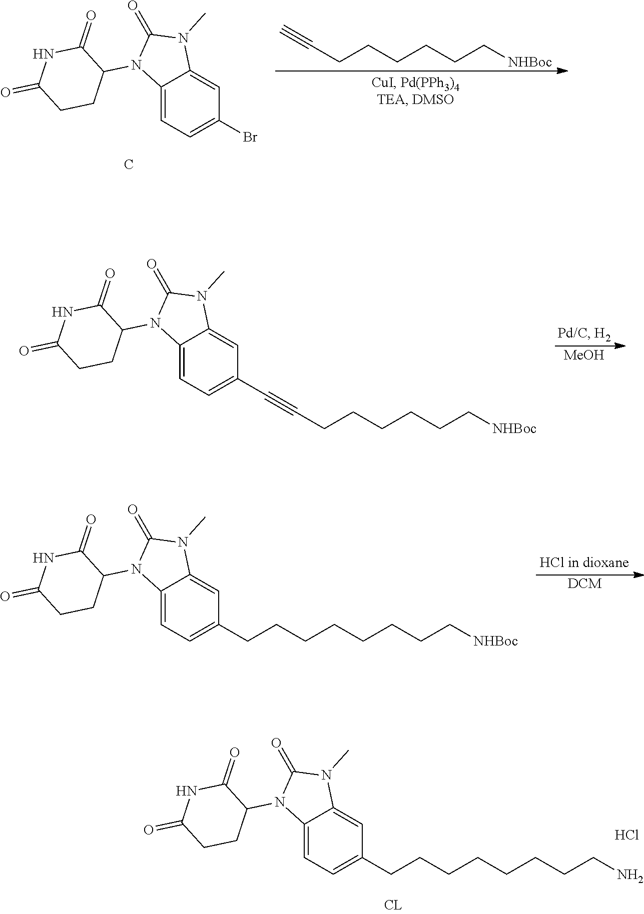

### Step 1—Tert-butyl N-[8-[1-(2,6-dioxopiperidin-3-yl)-3-methyl-2-oxo-1,3-benzodiazol-5-yl]oct-7-yn-1-yl]carbamate

To a stirred solution of tert-butyl N-(oct-7-yn-1-yl)carbamate (8.00 g, 35.5 mmol) and 3-(5-bromo-3-methyl-2-oxo-1,3-benzodiazol-1-yl)piperidine-2,6-dione (8.00 g, 23.7 mmol, Intermediate C) in DMSO (50.00 mL) and TEA (25.00 mL) were added Pd(PPh₃)₄ (1.71 g, 1.48 mmol) and CuI (281.60 mg, 1.479 mmol) in portions at rt under nitrogen atmosphere. The resulting mixture was stirred for 3 h at 80° C. under nitrogen atmosphere. On completion, the reaction mixture was diluted with EtOAc (800 mL). The resulting mixture was washed with water (4×400 mL). The resulting mixture was concentrated under reduced pressure. The residue was purified by silica gel column chromatography, eluted with PE/EtOAc (1:2), to afford the title compound (6 g, 84% yield) as a yellow solid. LC/MS (ESI, m/z): [(M+1)]⁺=483.2.

### Step 2—Tert-butyl N-[8-[1-(2,6-dioxopiperidin-3-yl)-3-methyl-2-oxo-1,3-benzodiazol-5-yl]octyl]carbamate

To a stirred solution of tert-butyl N-[8-[1-(2,6-dioxopiperidin-3-yl)-3-methyl-2-oxo-1,3-benzodiazol-5-yl]oct-7-yn-1-yl]carbamate (3.00 g, 6.22 mmol) in MeOH (100.00 mL) was added Pd/C (661.57 mg, 6.217 mmol, 10 wt %) in portions at rt under nitrogen atmosphere. The reaction system was degassed under vacuum and purged with H₂ several times, then it was hydrogenated under H₂ balloon (1 atm) at rt for 48 h. After completion of the reaction, Pd/C was filtered off through celite. The filter cake was washed with MeOH (3×100 mL). The corresponding filtrate was concentrated under reduced pressure to afford the title compound (2.47 g, 82% yield) as a yellow solid. LC/MS (ESI, m/z): [(M+1)]⁺=487.2.

### Step 3—3-[5-(8-Aminooctyl)-3-methyl-2-oxo-1,3-benzodiazol-1-yl]piperidine-2,6-dione Hydrochloride

To a stirred solution of tert-butyl N-[8-[1-(2,6-dioxopiperidin-3-yl)-3-methyl-2-oxo-1,3-benzodiazol-5-yl]octyl]carbamate (6.50 g, 13.4 mmol) in DCM (20.00 mL) was added 4 M HCl (gas) in 1,4-dioxane (20.00 mL) dropwise at 0° C. under nitrogen atmosphere. The resulting mixture was stirred for 4 h at rt under nitrogen atmosphere. The resulting mixture was concentrated under reduced pressure. The residue was triturated with Et₂O to afford the title compound (5.5 g, 97% yield) as a white solid. LC/MS (ESI, m/z): [(M+1)]⁺=387.2.

### Example 1.91. Preparation of N-[(1R,2S)-2-fluorocyclopropyl]-7-(methylamino)-5-{[2-oxo-1-(piperidin-4-yl)pyridin-3-yl]amino}pyrazolo[1,5-a]pyrimidine-3-carboxamide (Intermediate CM)

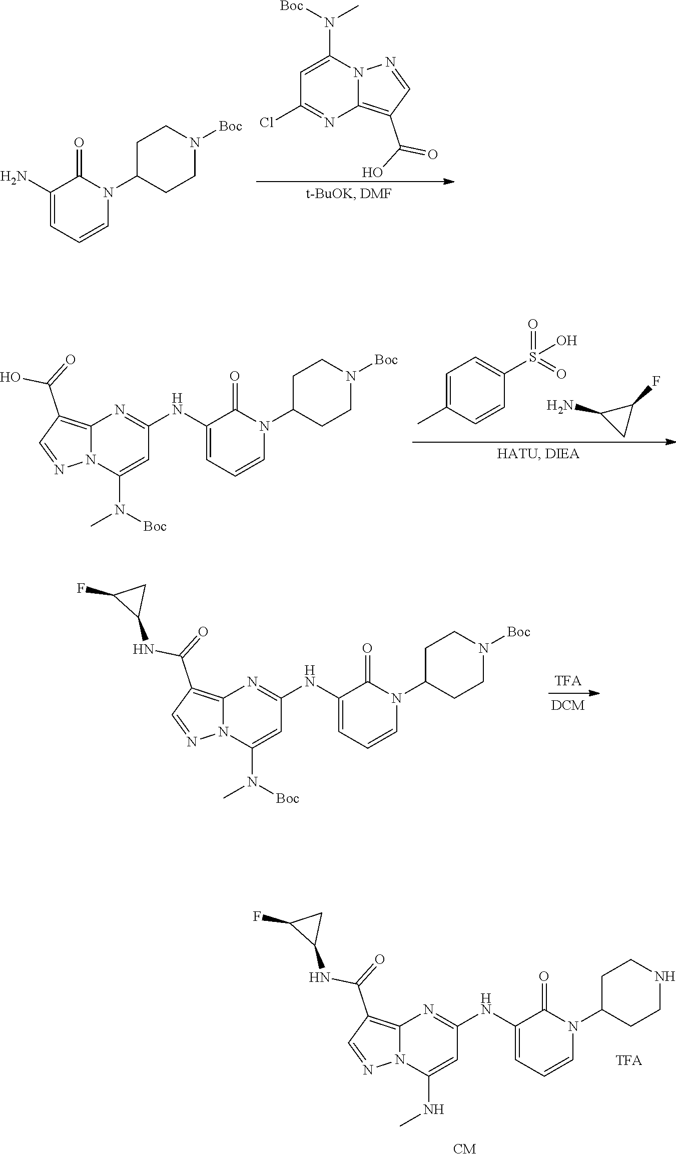

### Step 1—7-[(Tert-butoxycarbonyl)(methyl)amino]-5-({1-[1-(tert-butoxycarbonyl)piperidin-4-yl]-2-oxopyridin-3-yl}amino)pyrazolo[1,5-a]pyrimidine-3-carboxylic Acid

To a stirred solution of 7-[(tert-butoxycarbonyl)(methyl)amino]-5-chloropyrazolo[1,5-a]pyrimidine-3-carboxylic acid (300 mg, 0.9 mmol, CAS #2271470-66-5) and tert-butyl 4-(3-amino-2-oxopyridin-1-yl)piperidine-1-carboxylate (296.29 mg, 1.010 mmol, synthesized via Steps 1-2 of Intermediate AN) in DMF (6 mL) was added t-BuOK (2.30 mL, 2.30 mmol) dropwise at 0° C. under nitrogen atmosphere. The resulting mixture was stirred for 2 h at rt under nitrogen atmosphere. On completion, the reaction mixture was concentrated under reduced pressure. The residue was purified by reverse phase flash chromatography (Column: WelFlash™ C18-I, 20-40 μm, 120 g; Eluent A: Water (plus 10 mmol/L FA); Eluent B: ACN; Gradient: 30%-70% B in 35 min; Flow rate: 60 mL/min; Detector: 220/254 nm; desired fractions were collected at 65% B) and concentrated under reduced pressure to afford the title compound (230 mg, 43% yield) as an off-white solid. LC/MS (ESI, m/z): [(M+H)]⁺=584.3.

### Step 2—Tert-butyl 4-[3-({7-[(tert-butoxycarbonyl)(methyl)amino]-3-{[(1R,2S)-2-fluorocyclopropyl]carbamoyl}pyrazolo[1,5-a]pyrimidin-5-yl}amino)-2-oxopyridin-1-yl]piperidine-1-carboxylate

To a stirred solution of 7-[(tert-butoxycarbonyl)(methyl)amino]-5-({1-[1-(tert-butoxycarbonyl)piperidin-4-yl]-2-oxopyridin-3-yl}amino)pyrazolo[1,5-a]pyrimidine-3-carboxylic acid (223 mg, 0.382 mmol) and (1R,2S)-2-fluorocyclopropan-1-amine para-toluene sulfonate (944.81 mg, 3.820 mmol) in DMF (3 mL) were added HATU (217.92 mg, 0.573 mmol) and DIEA (0.20 mL, 1.146 mmol) in turns at rt. The resulting mixture was stirred for 2 h at rt under nitrogen atmosphere. On completion, the reaction mixture was concentrated under reduced pressure. The residue was purified by reverse phase flash chromatography (Column: WelFlash™ C18-I, 20-40 μm, 120 g; Eluent A: Water (plus 5 mmol/L NH₄HCO₃); Eluent B: ACN; Gradient: 20%-50% B in 35 min; Flow rate: 60 mL/min; Detector: 220/254 nm; desired fractions were collected at 32% B) and concentrated under reduced pressure to afford the title compound (150 mg, 61% yield) as an off-white solid. LC/MS (ESI, m/z): [(M+H)]⁺=641.5.

### Step 3—N-[(1R,2S)-2-fluorocyclopropyl]-7-(methylamino)-5-{[2-oxo-1-(piperidin-4-yl)pyridin-3-yl]amino}pyrazolo[1,5-a]pyrimidine-3-carboxamide Trifluoroacetate

To a stirred solution of tert-butyl 4-[3-({7-[(tert-butoxycarbonyl)(methyl)amino]-3-{[(1R,2S)-2-fluorocyclopropyl]carbamoyl}pyrazolo[1,5-a]pyrimidin-5-yl}amino)-2-oxopyridin-1-yl]piperidine-1-carboxylate (130 mg, 0.203 mmol) in DCM (2 mL) was added TFA (1.5 mL) dropwise at 0° C. under nitrogen atmosphere. The resulting mixture was stirred for 2 h at rt under nitrogen atmosphere. On completion, the reaction mixture was concentrated under reduced pressure. The residue was triturated with Et₂O to afford the title compound (100 mg) as an off-white solid. LC/MS (ESI, m/z): [(M+H)]⁺=441.1.

### Example 1.92. Preparation of 3-(5-{2,7-diazaspiro[3.5]nonan-7-yl}-3-methyl-2-oxo-1,3-benzodiazol-1-yl)piperidine-2,6-dione (Intermediate CN)

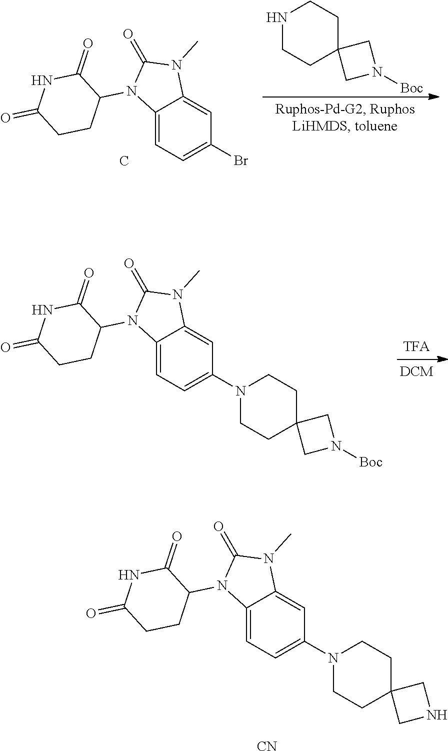

### Step 1—Tert-butyl 7-(1-(2,6-dioxopiperidin-3-yl)-3-methyl-2-oxo-2,3-dihydro-1H-benzo[d]imidazol-5-yl)-2,7-diazaspiro[3.5]nonane-2-carboxylate

To a solution of 3-(5-bromo-3-methyl-2-oxo-benzimidazol-1-yl)piperidine-2,6-dione (6 g, 18 mmol, Intermediate C), tert-butyl 2,7-diazaspiro[3.5]nonane-2-carboxylate (4.82 g, 21.3 mmol), [2-(2-aminophenyl)phenyl]-chloro-palladium; dicyclohexyl-[2-(2,6-diisopropoxyphenyl)phenyl]phosphane (2.76 g, 3.55 mmol), LiHMDS (1 M, 88.72 mL) and 4 Å molecular sieves (1.2 g) in toluene (120 mL) was added RuPhos (1.66 g, 3.55 mmol). The mixture was stirred at 80° C. under N₂ for 2 hr. On completion, the reaction mixture was adjusted to pH=5 with FA at 0° C., then filtered, and filtrate was concentrated under reduced pressure to give a residue. The residue was purified by column chromatography (SiO₂, PE:EA=2:1 to 0:1, P: Rf=0.4 (PE:EA=0:1)) to give the crude product (5 g, 83% purity). The crude product was then triturated with MTBE (20 mL) at 25° C. for 10 min and filtered, then washed ACN (10 mL×3), then the solid was dried under reduced pressure. The title compound (3.96 g, 42% yield) was obtained as off-white solid. ¹H NMR (400 MHz, CHLOROFORM-d) δ=8.34 (br s, 1H), 6.68 (s, 2H), 6.65 (s, 1H), 5.21-5.17 (dd, J=5.3, 12.8 Hz, 1H), 3.69 (s, 4H), 3.40 (s, 3H), 3.06-3.03 (m, 4H), 2.90 (m, 1H), 2.83-2.81 (m, 1H), 2.71-2.68 (m, 1H), 2.24-2.22 (m, 1H), 1.93-1.90 (m, 4H), 1.46 (s, 9H).

### Step 2—3-(5-{2,7-Diazaspiro[3.5]nonan-7-yl}-3-methyl-2-oxo-1,3-benzodiazol-1-yl)piperidine-2,6-dione Trifluoroacetate

To a stirred mixture of tert-butyl 7-[1-(2,6-dioxopiperidin-3-yl)-3-methyl-2-oxo-1,3-benzodiazol-5-yl]-2,7-diazaspiro[3.5]nonane-2-carboxylate (50 mg, 0.1 mmol) and TFA (0.6 mL) in DCM (3 mL) at rt under nitrogen atmosphere. The resulting mixture was stirred for 1 h at rt under nitrogen atmosphere. On completion, the reaction mixture was concentrated under reduced pressure. The residue was purified by trituration with ethyl ether (15 mL). The precipitated solids were collected by filtration and washed with ethyl ether (6 mL). The solid was dried under reduced pressure to afford the title compound (50 mg) as a white solid. LC/MS (ESI, m/z): [(M+H)]⁺=384.2.

### Example 1.93. Preparation of 3-(3-methyl-5-(methyl(piperidin-4-ylmethyl)amino)-2-oxo-2,3-dihydro-1H-benzo[d]imidazol-1-yl)piperidine-2,6-dione (Intermediate CO)

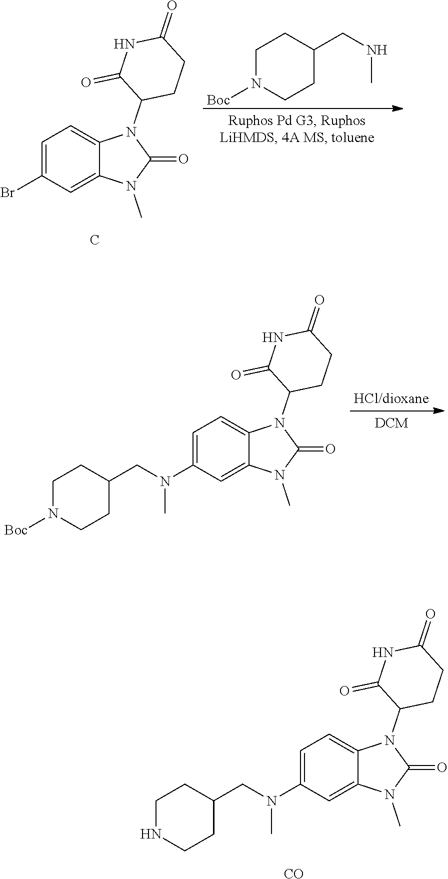

### Step 1—Tert-butyl 4-(((1-(2,6-dioxopiperidin-3-yl)-3-methyl-2-oxo-2,3-dihydro-1H-benzo[d]imidazol-5-yl)(methyl)amino)methyl)piperidine-1-carboxylate

To a solution of 3-(5-bromo-3-methyl-2-oxo-2,3-dihydro-1H-benzo[d]imidazol-1-yl)piperidine-2,6-dione (10 g, 30 mmol, Intermediate C), tert-butyl 4-((methylamino)methyl)piperidine-1-carboxylate (8.10 g, 35.4 mmol), RuPhos Pd G3 (1.24 g, 1.48 mmol), RuPhos (689 mg, 1.48 mmol) and 4 Å molecular sieves (10 g) in toluene (150 mL). Then LiHMDS (1 M, 147.86 mL) was added to the mixture which was stirred at 100° C. for 12 h under N₂. On completion, the reaction mixture was filtered and diluted with H₂O (100 mL), and extracted with EtOAc (100 mL×3). The combined organic layers were washed with brine (100 mL×3), dried over Na₂SO₄, filtered and concentrated in vacuo. The residue was purified by column chromatography (SiO₂, Petroleum ether/Ethyl acetate=3/1 to 0/1) to give the title compound (8 g, 56% yield) as brown oil. LC-MS (ESI⁺) m/z 485.9 (M+H)⁺.

### Step 2—3-(3-methyl-5-(methyl(piperidin-4-ylmethyl)amino)-2-oxo-2,3-dihydro-1H-benzo[d]imidazol-1-yl)piperidine-2,6-dione

A mixture of tert-butyl 4-(((1-(2,6-dioxopiperidin-3-yl)-3-methyl-2-oxo-2,3-dihydro-1H-benzo[d]imidazol-5-yl)(methyl)amino)methyl)piperidine-1-carboxylate (4 g, 8 mmol), HCl/dioxane (4 M, 2.06 mL) in DCM (30 mL) was degassed and purged with N₂ three times, and then the mixture was stirred at 25° C. for 2 h under N₂ atmosphere. On completion, concentrated in vacuo to give the title compound (4 g) as a white solid. LC-MS (ESI⁺) m/z 386.1 (M+H)⁺.

### Example 1.94. Preparation of 3-[5-(Azetidin-3-yl)-3-methyl-2-oxo-benzimidazol-1-yl]piperidine-2,6-dione (Intermediate CP)

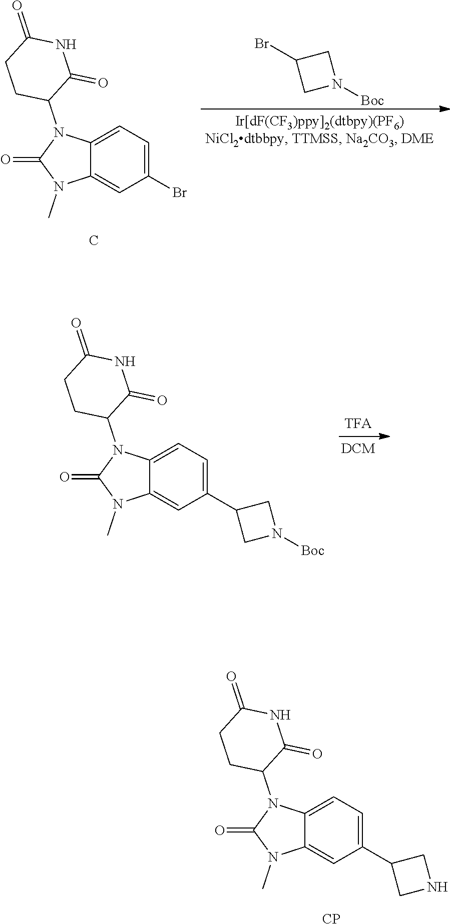

### Step 1—Tert-butyl 3-[1-(2,6-dioxo-3-piperidyl)-3-methyl-2-oxo-benzimidazol-5-yl]azetidine-1-carboxylate

To an vial equipped with a stir bar was added 3-(5-bromo-3-methyl-2-oxo-benzimidazol-1-yl)piperidine-2,6-dione (4.00 g, 11.8 mmol, Intermediate C), tert-butyl 3-bromoazetidine-1-carboxylate (3.63 g, 15.3 mmol, CAS #1064194-10-0), Ir[dF(CF₃)ppy]₂(dtbpy)(PF₆) (1.33 g, 1.18 mmol), NiCl₂.dtbbpy (23.5 mg, 59.1 umol), TTMSS (2.94 g, 11.8 mmol), and sodium carbonate (2.51 g, 23.6 mmol) in 1,2-dimethoxyethane (100 mL). The vial was sealed and placed under nitrogen was added. The reaction was stirred and irradiated with a 34 W blue LED lamp (7 cm away), with cooling fan to keep the reaction temperature at 25° C. for 14 hr. On completion, the reaction mixture was filtered and concentrated under reduced pressure to give a residue. The crude product was triturated with ethyl acetate 20 mL at 25° C. for 30 min to give the title compound (2.38 g, 47% yield) as a white solid. LC-MS (ESI+) m/z 414.9 (M+H)⁺.

### Step 2 3-[5-(Azetidin-3-yl)-3-methyl-2-oxo-benzimidazol-1-yl]piperidine-2,6-dione

To a solution of tert-butyl 3-[1-(2,6-dioxo-3-piperidyl)-3-methyl-2-oxo-benzimidazol-5-yl]azetidine-1-carboxylate (2.10 g, 5.07 mmol) in dichloromethane (30 mL) was added trifluoroacetic acid (7.70 g, 67.5 mmol). The mixture was stirred at 25° C. for 0.5 hr. On completion, the mixture was concentrated in vacuo to give the title compound (1.6 g) as yellow oil. LC-MS (ESI+) m/z 315.0 (M+H)⁺.

### Example 1.95. Preparation of 3-{3-Methyl-2-oxo-5-[1-(piperidin-4-yl)azetidin-3-yl]-1,3-benzodiazol-1-yl}piperidine-2,6-dione (Intermediate CQ)

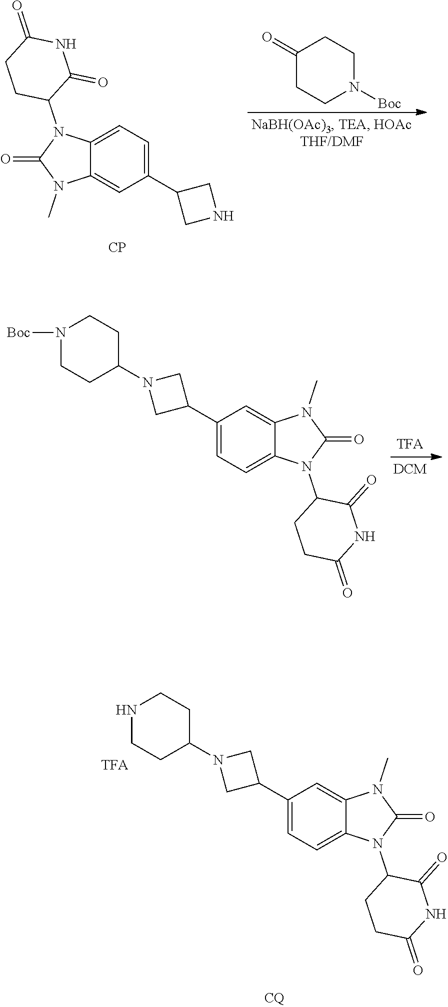 [SMILES: Cn1c(=O)n(C2CCC(=O)NC2=O)c2ccc(C3CNC3)cc21 ; CC(C)(C)OC(=O)N1CCC(=O)CC1 ; "CP" ; Cn1c(=O)n(C2CCC(=O)NC2=O)c2ccc(C3CN(C4CCN(C(=O)OC(C)(C)C)CC4)C3)cc21 ; Cn1c(=O)n(C2CCC(=O)NC2=O)c2ccc(C3CN(C4CCNCC4)C3)cc21]

### Step 1—Tert-butyl 4-[3-[1-(2,6-dioxo-3-piperidyl)-3-methyl-2-oxo-benzimidazol-5-yl]azetidin-1-yl]piperidine-1-carboxylate

To a solution of 3-[5-(azetidin-3-yl)-3-methyl-2-oxo-benzimidazol-1-yl]piperidine-2,6-dione (1.60 g, 5.09 mmol, Intermediate CP) in tetrahydrofuran (50 mL) and dimethylformamide (10 mL) was added triethylamine (515 mg, 5.09 mmol) and acetic acid (305 mg, 5.09 mmol). Then, tert-butyl 4-oxopiperidine-1-carboxylate (1.22 g, 6.11 mmol) was added to the mixture, the mixture was stirred at 0-25° C. for 0.5 hour. Sodium triacethoxy borohydride (1.08 g, 5.09 mmol) was added and the mixture was stirred at 0° C. for 1.5 hour. On completion, the reaction mixture was quenched with water (20 mL) at 0° C., and then saturated sodium bicarbonate was added until the pH=7-8. The solid precipitate was then filtered, and the cake was washed water (20 mL×3). The filtered cake was dried in vacuo to give the title compound (1.80 g) as a light yellow solid. ¹H NMR (400 MHz, DMSO-d6) δ=11.10 (s, 1H), 7.30 (s, 1H), 7.16-7.03 (m, 2H), 5.37 (dd, J=5.2, 12.4 Hz, 1H), 4.61-4.21 (m, 2H), 4.21-3.81 (m, 4H), 3.37 (s, 3H), 2.97-2.84 (m, 1H), 2.82-2.58 (m, 4H), 2.52 (s, 3H), 2.04-1.90 (m, 2H), 1.40 (s, 9H), 1.25-1.14 (m, 2H); LC-MS (ESI+) m/z 498.2 (M+H)⁺.

### Step 2—3-{3-Methyl-2-oxo-5-[1-(piperidin-4-yl)azetidin-3-yl]-1,3-benzodiazol-1-yl}piperidine-2,6-dione Trifluoroacetate

To a stirred solution of tert-butyl 4-{3-[1-(2,6-dioxopiperidin-3-yl)-3-methyl-2-oxo-1,3-benzodiazol-5-yl]azetidin-1-yl}piperidine-1-carboxylate (75 mg, 0.15 mmol) in DCM (3 mL) was added TFA (1 mL) dropwise at rt. The resulting mixture was stirred for 1 h at rt. On completion, the reaction mixture was concentrated under reduced pressure. The residue was triturated with Et₂O to afford the title compound (50 mg) as a white solid. LC/MS (ESI, m/z): [(M+H)]⁺=398.2.

### Example 1.96. Preparation of 3-(3-methyl-4-{[4-(methylamino)piperidin-1-yl]methyl}-2-oxo-1,3-benzodiazol-1-yl)piperidine-2,6-dione (Intermediate CR)

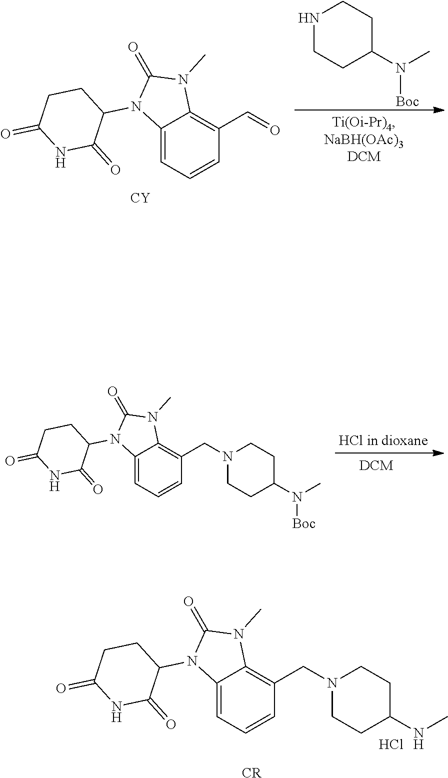

### Step 1—Tert-butyl N-(1-[[1-(2,6-dioxopiperidin-3-yl)-3-methyl-2-oxo-1,3-benzodiazol-4-yl]methyl]piperidin-4-yl)-N-methylcarbamate

To a stirred mixture of 1-(2,6-dioxopiperidin-3-yl)-3-methyl-2-oxo-1,3-benzodiazole-4-carbaldehyde (5.00 g, 17.4 mmol, Intermediate CY) and tert-butyl N-methyl-N-(piperidin-4-yl)carbamate (4.48 g, 20.9 mmol, CAS #108612-54-0) in DCM (100.00 mL) was added Ti(Oi-Pr)₄ (14.84 g, 52.21 mmol) dropwise at rt. The resulting mixture was stirred for 10 min at rt under air atmosphere. To the above mixture was added NaBH(OAc)₃ (7.38 g, 34.8 mmol) in portions over 10 min at rt. The resulting mixture was stirred for additional 16 h at rt. On completion, the reaction was quenched by the addition of water (50 mL) at rt. The resulting mixture was filtered, the filter cake was washed with methanol (3×50 mL). The filtrate was concentrated under reduced pressure. The resulting mixture was extracted with CH₂Cl₂ (2×50 mL). The combined organic layers were washed with brine (30 mL), dried over anhydrous Na₂SO₄. After filtration, the filtrate was concentrated under reduced pressure. The residue was purified by trituration with ethyl ether (20 mL) to give the title compound (3.5 g, 41% yield) as a brown solid. LC/MS (ESI, m/z): [(M+1)]⁺=486.3.

### Step 2—3-(3-Methyl-4-{[4-(methylamino)piperidin-1-yl]methyl}-2-oxo-1,3-benzodiazol-1-yl)piperidine-2,6-dione Hydrochloride

To a stirred solution of tert-butyl N-(1-{[1-(2,6-dioxopiperidin-3-yl)-3-methyl-2-oxo-1,3-benzodiazol-4-yl]methyl}piperidin-4-yl)-N-methylcarbamate (2 g, 4 mmol) in dioxane (20.00 mL) was added 1,4-dioxane (20 mL) dropwise at rt. The resulting mixture was stirred for 2 h at rt. On completion, the precipitated solids were collected by filtration and washed with ethyl ether (20 mL) to give the title compound (1.5 g, 80% yield) as a brown solid. LC/MS (ESI, m/z): [(M+1)]⁺=386.3.

### Example 1.97. Preparation of 3-{3-Methyl-2-oxo-4-[3-(piperidin-4-yl)prop-1-yn-1-yl]-1,3-benzodiazol-1-yl}piperidine-2,6-dione (Intermediate CS)

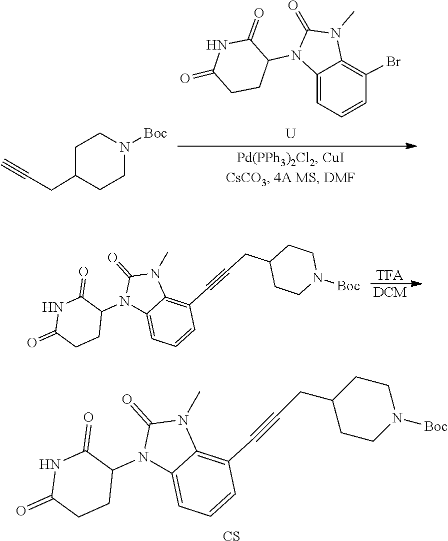

### Step 1—Tert-butyl 4-[3-[1-(2,6-dioxo-3-piperidyl)-3-methyl-2-oxo-benzimidazol-4-yl]prop-2-ynyl] piperidine-1-carboxylate

To a mixture of 3-(4-bromo-3-methyl-2-oxo-benzimidazol-1-yl)piperidine-2,6-dione (1.00 g, 2.96 mmol, Intermediate U) and tert-butyl 4-prop-2-ynylpiperidine-1-carboxylate (990 mg, 4.44 mmol, CAS #301185-41-1) in DMF (20 mL) was added Cs₂CO₃ (4.82 g, 14.7 mmol), CuI (168 mg, 887 umol), Pd(PPh₃)₂Cl₂ (622 mg, 887 umol) and 4 Å molecular sieves (200 mg) under N₂. The reaction mixture was then stirred at 80° C. for 2 hours. On completion, the reaction mixture was filtered, the filtrate was poured into water (100 mL), and the aqueous phase was extracted with ethyl acetate (2×40 mL). The combined organic phase was washed with brine (2×40 mL), dried over anhydrous Na₂SO₄, filtered and concentrated in vacuo to give the crude compound. The crude product was purified by reversed-phase HPLC (0.1% FA condition) to give the title compound (1.20 g, 84% yield) as a brown solid. LC/MS (ESI, m/z): [(M+Na)]+=503.4.

### Step 2—3-{3-Methyl-2-oxo-4-[3-(piperidin-4-yl)prop-1-yn-1-yl]-1,3-benzodiazol-1-yl}piperidine-2,6-dione Trifluoroacetate

To a stirred solution of tert-butyl 4-{3-[1-(2,6-dioxopiperidin-3-yl)-3-methyl-2-oxo-1,3-benzodiazol-4-yl]prop-2-yn-1-yl}piperidine-1-carboxylate (75 mg, 0.16 mmol) in DCM (3 mL) was added TFA (1 mL) at rt. The resulting mixture was stirred for 2 h at rt. On completion, the reaction mixture was concentrated under reduced pressure. The residue was triturated with Et₂O to afford the title compound (60 mg) as a white solid. LC/MS (ESI, m/z): [(M+H)]⁺=381.1.

### Example 1.98. Preparation of 3-{3-methyl-2-oxo-4-[3-(piperazin-1-yl)prop-1-yn-1-yl]-1,3-benzodiazol-1-yl}piperidine-2,6-dione (Intermediate CT)

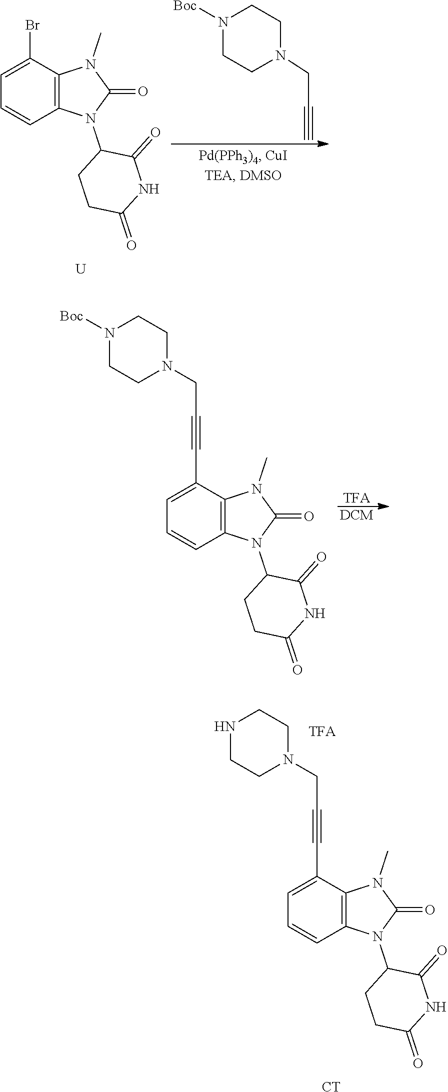

### Step 1—Tert-butyl 4-{3-[1-(2,6-dioxopiperidin-3-yl)-3-methyl-2-oxo-1,3-benzodiazol-4-yl]prop-2-yn-1-yl}piperazine-1-carboxylate

To a stirred solution of 3-(4-bromo-3-methyl-2-oxo-2,3-dihydro-1H-benzo[d]imidazol-1-yl)piperidine-2,6-dione (1 g, 4 mmol, Intermediate U) and tert-butyl 4-(prop-2-yn-1-yl)piperazine-1-carboxylate (1.42 g, 6.32 mmol) in DMSO (20 mL) were added CuI (80.27 mg, 0.421 mmol), TEA (7 mL) and Pd(PPh₃)₄ (487.06 mg, 0.421 mmol) in turns at rt. The resulting mixture was stirred for 3 h at 90° C. On completion, the reaction mixture was cooled to rt and concentrated under reduced pressure. The residue was purified by reverse flash chromatography (Column: WelFlash™ C18-I, 20-40 um, 330 g; Eluent A: Water (plus 10 mmol/L NH₄HCO₃); Eluent B: ACN; Gradient: 35%-55% B in 20 min; Flow rate: 80 mL/min; Detector: 254 nm; desired fractions were collected at 40% B) and concentrated under reduced pressure to afford the title compound (1.2 g, 59% yield) as a white solid. LC/MS (ESI, m/z): [(M+H)]⁺=482.2.

### Step 2—3-{3-Methyl-2-oxo-4-[3-(piperazin-1-yl)prop-1-yn-1-yl]-1,3-benzodiazol-1-yl}piperidine-2,6-dione Trifluoroacetate

To a stirred solution of tert-butyl 4-{3-[1-(2,6-dioxopiperidin-3-yl)-3-methyl-2-oxo-1,3-benzodiazol-4-yl]prop-2-yn-1-yl}piperazine-1-carboxylate (1.2 g, 2.492 mmol) in DCM (20 mL) was added TFA (7 mL) at rt. The resulting mixture was stirred for 2 h at rt. On completion, the reaction mixture was concentrated under reduced pressure. The residue was triturated with Et₂O to afford the title compound (1.1 g) as a white solid. LC/MS (ESI, m/z): [(M+H)]⁺=382.2.

### Example 1.99. Preparation of 3-{3-methyl-2-oxo-5-[4-(piperidin-4-yl)piperazin-1-yl]-1,3-benzodiazol-1-yl}piperidine-2, 6-dione (Intermediate CU)

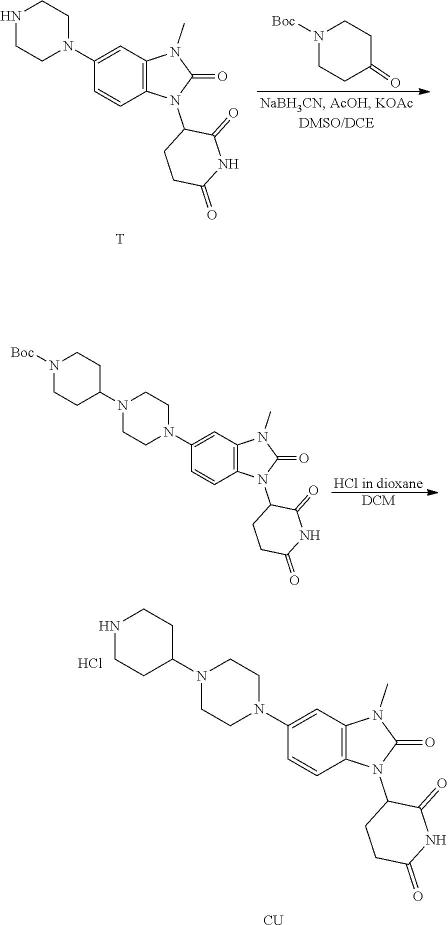

### Step 1—Tert-butyl 4-{4-[1-(2,6-dioxopiperidin-3-yl)-3-methyl-2-oxo-1,3-benzodiazol-5-yl]piperazin-1-yl}piperidine-1-carboxylate

To a stirred solution of 3-[3-methyl-2-oxo-5-(piperazin-1-yl)-1,3-benzodiazol-1-yl]piperidine-2,6-dione (500 mg, 1.456 mmol, Intermediate T) and tert-butyl 4-oxopiperidine-1-carboxylate (348.15 mg, 1.747 mmol) in DCE (5 mL) and DMSO (5 mL) were added KOAc (428.71 mg, 4.368 mmol), AcOH (0.2 mL, 0.349 mmol) in turns at rt. The resulting mixture was stirred for 30 min at rt. To the above mixture was added NaBH₃CN (457.50 mg, 7.280 mmol) at 0° C. The resulting mixture was stirred for additional 2 h at rt. On completion, the reaction mixture was concentrated under reduced pressure. The residue was purified by reverse phase Flash chromatography (Column: WelFlash™ C18-I, 20-40 um, 120 g; Eluent A: Water (plus 10 mmol/L FA); Eluent B: ACN; Gradient: 30%-70% B in 30 min; Flow rate: 60 mL/min; Detector: 254 nm; desired fractions were collected at 50% B) and concentrated under reduced pressure to afford the title compound (260 mg, 34% yield) as a white solid. LC/MS (ESI, m/z): [(M+H)]⁺=527.3.

### Step 2—3-{3-methyl-2-oxo-5-[4-(piperidin-4-yl)piperazin-1-yl]-1,3-benzodiazol-1-yl}piperidine-2,6-dione Hydrochloride

To a stirred solution of tert-butyl 4-{4-[1-(2,6-dioxopiperidin-3-yl)-3-methyl-2-oxo-1,3-benzodiazol-5-yl]piperazin-1-yl}piperidine-1-carboxylate (260 mg, 0.494 mmol) in DCM (5 mL) was added 4 M HCl (gas) in 1,4-dioxane (1 mL) at rt. The resulting mixture was stirred for 2 h at rt. On completion, the reaction mixture was concentrated under reduced pressure. The residue was triturated with Et₂O to afford the title compound (220 mg) as a white solid. LC/MS (ESI, m/z): [(M+H)]⁺=427.2.

### Example 1.100. Preparation of 3-[5-(4-{[1,4′-Bipiperidin]-4-yl}piperazin-1-yl)-3-methyl-2-oxo-1,3-benzodiazol-1-yl]piperidine-2,6-dione (Intermediate CV)

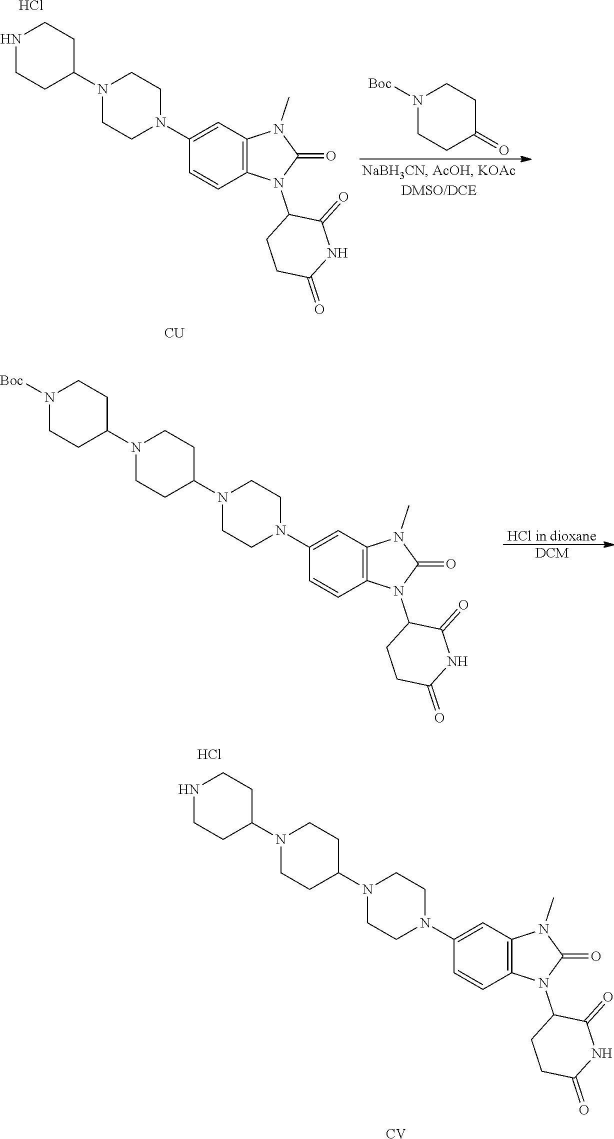

### Step 1—Tert-butyl 4-{4-[1-(2,6-dioxopiperidin-3-yl)-3-methyl-2-oxo-1,3-benzodiazol-5-yl]piperazin-1-yl}-[1,4′-bipiperidine]-1′-carboxylate

To a stirred solution of 3-{3-methyl-2-oxo-5-[4-(piperidin-4-yl)piperazin-1-yl]-1,3-benzodiazol-1-yl}piperidine-2,6-dione hydrochloride (220 mg, 0.48 mmol, Intermediate CU) and tert-butyl 4-oxopiperidine-1-carboxylate (113.62 mg, 0.570 mmol) in DCE (5 mL) and DMSO (5 mL) were added KOAc (139.91 mg, 1.425 mmol) and AcOH (0.12 mL, 0.349 mmol) in turns at rt. The resulting mixture was stirred for 30 min at rt. To the above mixture was added NaBH₃CN (149.30 mg, 2.375 mmol) at 0° C. The resulting mixture was stirred for additional 2 h at rt. On completion, the reaction mixture was concentrated under reduced pressure. The residue was purified by reverse phase flash chromatography (Column: WelFlash™ C18-I, 20-40 um, 120 g; Eluent A: Water (plus 10 mmol/L FA); Eluent B: ACN; Gradient: 40%-70% B in 40 min; Flow rate: 60 mL/min; Detector: 254 nm; desired fractions were collected at 53% B) and concentrated under reduced pressure to afford the title compound (140 mg, 48% yield) as a white solid. LC/MS (ESI, m/z): [(M+H)]⁺=610.4.

### Step 2—3-[5-(4-{[1,4′-Bipiperidin]-4-yl}piperazin-1-yl)-3-methyl-2-oxo-1,3-benzodiazol-1-yl]piperidine-2,6-dione hydrochloride

To a stirred solution of tert-butyl 4-{4-[1-(2,6-dioxopiperidin-3-yl)-3-methyl-2-oxo-1,3-benzodiazol-5-yl]piperazin-1-yl}-[1,4′-bipiperidine]-1′-carboxylate (140 mg, 0.23 mmol) in DCM (3 mL) was 4 M HCl (gas) in 1,4-dioxane (1 mL) added at rt. The resulting mixture was stirred for 2 h at rt. On completion, the reaction mixture was concentrated under reduced pressure. The residue was triturated with Et₂O to afford the title compound (110 mg) as a white solid. LC/MS (ESI, m/z): [(M+H)]⁺=510.2.

### Example 1.101. Preparation of 3-[5-(4-{2,7-diazaspiro[4.4]nonan-2-yl}phenyl)-3-methyl-2-oxo-1,3-benzodiazol-1-yl]piperidine-2,6-dione (Intermediate CW)

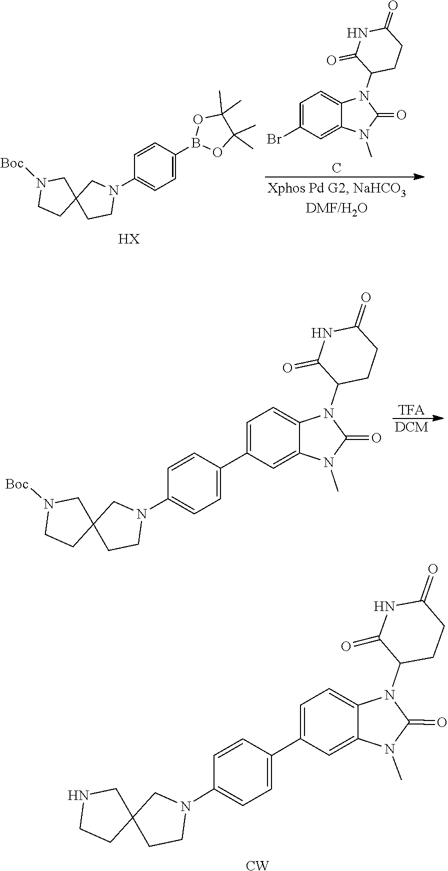

### Step 1—Tert-butyl 7-(4-(1-(2,6-dioxopiperidin-3-yl)-3-methyl-2-oxo-2,3-dihydro-1H-benzo[d]imidazol-5-yl)phenyl)-2,7-diazaspiro[4.4]nonane-2-carboxylate

A mixture of tert-butyl 7-(4-(4,4,5,5-tetramethyl-1,3,2-dioxaborolan-2-yl)phenyl)-2,7-diazaspiro[4.4]nonane-2-carboxylate (2.4 g, 5.6 mmol, Intermediate HX), 3-(5-bromo-3-methyl-2-oxo-2,3-dihydro-1H-benzo[d]imidazole-1-yl)piperidine-2,6-dione (1.71 g, 5.04 mmol, Intermediate C), XPHOS-PD-G2 (440 mg, 560 umol) and NaHCO₃ (941 mg, 11.2 mmol, 435.79 uL) in DMF (24 mL) and H₂O (2.4 mL) was stirred at 100° C. for 3 h under N₂ protection. On completion, the reaction mixture was quenched with H₂O (20 mL) at 20° C., and extracted with EtOAc (20 mL×3). The combined organic layers were washed with brine (20 mL×2), dried over Na₂SO₄, filtered and concentrated under reduced pressure to give a residue. The residue was purified by prep-HPLC (column: Phenomenex luna C18 250*50 mm*10 um; mobile phase: [water (FA)-ACN]; B %: 30%-60%, 20 min) to give the title compound (1 g, 32% yield) as white solid. ¹H NMR (400 MHz, DMSO-d₆) δ=11.10 (s, 1H), 7.52 (d, J=8.4 Hz, 2H), 7.38 (d, J=1.2 Hz, 1H), 7.24 (dd, J=1.6, 8.4 Hz, 1H), 7.11 (d, J=8.4 Hz, 1H), 6.60 (d, J=8.8 Hz, 2H), 5.37 (dd, J=5.2, 12.8 Hz, 1H), 3.43-3.33 (m, 7H), 3.28-3.18 (m, 4H), 2.99-2.85 (m, 1H), 2.78-2.60 (m, 2H), 2.09-1.82 (m, 5H), 1.40 (br d, J=5.6 Hz, 9H).

### Step 2—3-[5-(4-{2,7-Diazaspiro[4.4]nonan-2-yl}phenyl)-3-methyl-2-oxo-1,3-benzodiazol-1-yl]piperidine-2,6-dione Trifluoroacetate

To a stirred solution of tert-butyl 7-{4-[1-(2,6-dioxopiperidin-3-yl)-3-methyl-2-oxo-1,3-benzodiazol-5-yl]phenyl}-2,7-diazaspiro[4.4]nonane-2-carboxylate (45 mg, 0.080 mmol) in DCM (1 mL) was added TFA (0.2 mL) dropwise at rt. The resulting mixture was stirred for 1 h at rt under nitrogen atmosphere. On completion, the reaction mixture was concentrated under reduced pressure. The residue was purified by trituration with Et₂O to give the title compound (44 mg) as a white solid. LC/MS (ESI, m/z): [(M+1)]⁺=460.3.

### Example 1.102. Preparation of 3-{3-methyl-5-[(4-{[methyl({[(1r,4r)-4-aminocyclohexyl]methyl})amino]methyl}piperidin-1-yl)methyl]-2-oxo-1,3-benzodiazol-1-yl}piperidine-2,6-dione (Intermediate CX)

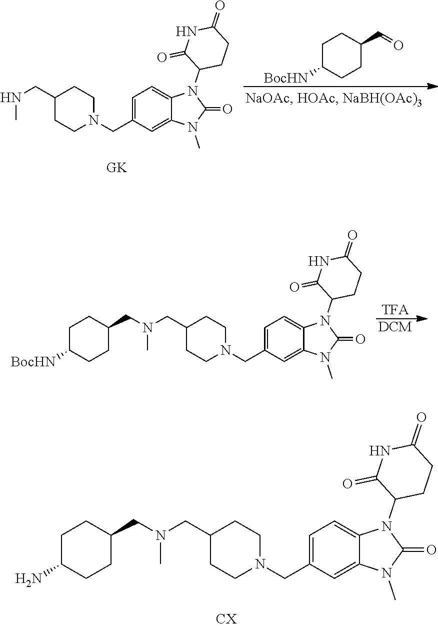 [SMILES: CNCC1CCN(Cc2ccc3c(c2)n(C)c(=O)n3C2CCC(=O)NC2=O)CC1 ; CC(C)(C)OC(=O)N[C@H]1CC[C@H](C=O)CC1 ; CN(CC1CCN(Cc2ccc3c(c2)n(C)c(=O)n3C2CCC(=O)NC2=O)CC1)C[C@H]1CC[C@H](NC(=O)OC(C)(C)C)CC1 ; CN(CC1CCN(Cc2ccc3c(c2)n(C)c(=O)n3C2CCC(=O)NC2=O)CC1)C[C@H]1CC[C@H](N)CC1]

### Step 1—Tert-butyl N-[4-[[[1-[[1-(2,6-dioxo-3-piperidyl)-3-methyl-2-oxo-benzimidazol-5-yl]methyl]-4-piperidyl]methyl-methyl-amino]methyl]cyclohexyl]carbamate

To a solution of 3-[3-methyl-5-[[4-(methylaminomethyl)-1-piperidyl]methyl]-2-oxo-benzimidazol-1-yl]piperidine-2,6-dione (2.86 g, 6.56 mmol, Intermediate GK, HCl) in dichloromethane (35 mL) and isopropyl alcohol (70 mL) was added sodium acetate (1.61 g, 19.7 mmol) at 25° C. and the mixture was stirred for 30 minutes. Then to the mixture was added tert-butyl N-(4-formylcyclohexyl)carbamate (2.24 g, 9.84 mmol) and acetic acid (788 mg, 13.1 mmol) at 25° C. and the mixture was stirred for another 30 minutes. Next, to the reaction mixture was added sodium triacethoxy borohydride (2.78 g, 13.12 mmol) at 0° C. Then, the mixture was stirred at 25° C. for 11 hr. On completion, the reaction mixture was quenched with 1 N sodium bicarbonate until the pH=8 at 25° C., then diluted with water (150 mL) and extracted with ethyl acetate (100 mL×3). The combined organic layers were washed with brine (100 mL×3), dried over anhydrous sodium sulfate, filtered and concentrated under reduced pressure to give a residue. The residue was purified by reversed-phase HPLC (0.1% FA condition). The resulting product was triturated with ethyl acetate at 25° C. for 30 minutes, filtered and the filter cake was dried in vacuo to give the title compound (1.38 g, 33% yield) as a white solid. ¹H NMR (400 MHz, DMSO-d6) δ=11.11 (s, 1H), 7.12 (s, 1H), 7.08-7.03 (m, 1H), 7.01-6.96 (m, 1H), 6.69 (d, J=8.0 Hz, 1H), 5.39-5.34 (m, 1H), 3.56 (s, 2H), 3.34 (s, 3H), 3.21-3.06 (m, 1H), 2.96-2.82 (m, 3H), 2.79-2.57 (m, 2H), 2.10-2.08 (m, 4H), 2.06-1.98 (m, 6H), 1.77-1.64 (m, 6H), 1.36 (s, 9H), 1.16-1.02 (m, 5H), 0.87-0.76 (m, 3H); LC-MS (ESI+) m/z 611.5 (M+H)⁺.

### Step 2—3-{3-Methyl-5-[(4-{[methyl({[(1r,4r)-4-aminocyclohexyl]methyl})amino]methyl}piperidin-1-yl)methyl]-2-oxo-1,3-benzodiazol-1-yl}piperidine-2,6-dione trifluoroacetate

To a stirred solution of tert-butyl N-[(1r,4r)-4-({[(1-{[1-(2,6-dioxopiperidin-3-yl)-3-methyl-2-oxo-1,3-benzodiazol-5-yl]methyl}piperidin-4-yl)methyl](methyl)amino}methyl)cyclohexyl]carbamate (45 mg, 0.074 mmol, KLT-K-017) in DCM (1 mL) was added TFA (0.2 mL) dropwise at rt under nitrogen atmosphere. The resulting mixture was stirred for 1 h at rt under nitrogen atmosphere. On completion, the reaction mixture was concentrated under reduced pressure. The residue was triturated with Et₂O to give the title compound (45 mg) as a white solid. LC/MS (ESI, m/z): [(M+1)]⁺=511.4.

### Example 1.103. Preparation of 1-(2,6-dioxopiperidin-3-yl)-3-methyl-2-oxo-1,3-benzodiazole-4-carbaldehyde (Intermediate CY)

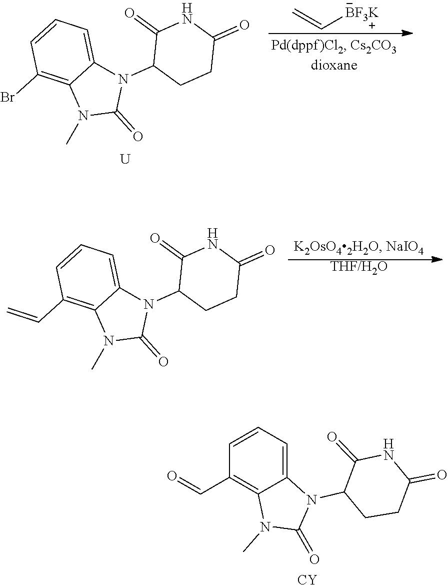

### Step 1—3-(4-Ethenyl-3-methyl-2-oxo-1,3-benzodiazol-1-yl)piperidine-2,6-dione

To a stirred mixture of 3-(4-bromo-3-methyl-2-oxo-1,3-benzodiazol-1-yl)piperidine-2,6-dione (3 g, 9 mmol, Intermediate U) and potassium ethenyldifluoroborane fluoride (3.57 g, 26.6 mmol) in dioxane (30 mL) were added Pd(dppf)Cl₂ (0.65 g, 0.89 mmol) and Cs₂CO₃ (5.78 g, 17.7 mmol) at rt under nitrogen atmosphere. The resulting mixture was stirred for 1 h at 80° C. under nitrogen atmosphere. On completion, the reaction mixture was cooled to rt and concentrated under reduced pressure. The residue was purified by silica gel column chromatography, eluted with PE/EA (5:1), to afford the title compound (1.4 g, 55% yield) as a yellow solid. LC/MS (ESI, m/z): [(M+1)]⁺=286.1.

### Step 2—1-(2,6-Dioxopiperidin-3-yl)-3-methyl-2-oxo-1,3-benzodiazole-4-carbaldehyde

To a stirred mixture of 3-(4-ethenyl-3-methyl-2-oxo-1,3-benzodiazol-1-yl)piperidine-2,6-dione (1.4 g, 4.9 mmol) and K₂OsO₄·2H₂O (0.09 g, 0.25 mmol) in THF (7 mL) and H₂O (7 mL) was added NaIO₄ (2.62 g, 12.3 mmol) at rt under nitrogen atmosphere. The resulting mixture was stirred for 1 h at rt under nitrogen atmosphere. The reaction was quenched with sat. sodium hyposulfite (aq.) at 0° C. The aqueous layer was extracted with EtOAc (3×10 mL). The combined organic layers were concentrated under vacuum to afford the title compound (370 mg, 26% yield) as a yellow solid. LC/MS (ESI, m/z): [(M+1)]⁺=288.1.

### Example 1.104. Preparation of 3-((3-(cyclobutylcarbamoyl)-8-(methylamino)imidazo[1,2-b]pyridazin-6-yl)amino)-2-oxo-2H-[1,2′-bipyridine]-5′-carboxylic Acid (Intermediate CZ)

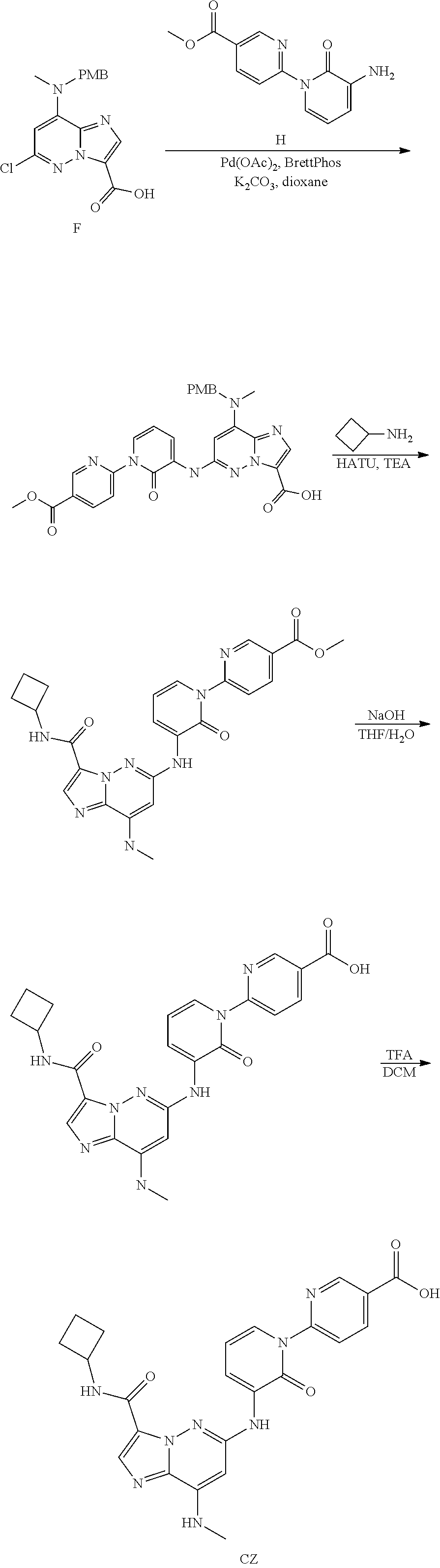

### Step 1—8-((4-Methoxybenzyl)(methyl)amino)-6-((5′-(methoxycarbonyl)-2-oxo-2H-[1,2′-bipyridin]-3-yl)amino)imidazo[1,2-b]pyridazine-3-carboxylic Acid

To a stirred solution of 6-chloro-8-{[(4-methoxyphenyl)methyl](methyl)amino}imidazo[1,2-b]pyridazine-3-carboxylic acid (2.3 g, 6.66 mmol, Intermediate F) and methyl 3-amino-2-oxo-[1,2′-bipyridine]-5′-carboxylate (1.63 g, 6.66 mmol, Intermediate H) in dioxane (40 mL) were added K₂CO₃ (1.83 g, 13.3 mmol), BrettPhos (0.60 g, 0.66 mmol) and Pd(OAc)₂ (0.16 g, 0.68 mmol) in turns at rt under nitrogen atmosphere. The resulting mixture was stirred for 4 h at 80° C. under nitrogen atmosphere. On completion, the reaction mixture was cooled to rt and acidified to pH 3 with conc. HCl and diluted with water (200 mL), then extracted with EtOAc (3×200 mL). The combined organic layers were washed with brine (3×200 mL), and dried over anhydrous Na₂SO₄. After filtration, the filtrate was concentrated under reduced pressure to give the title compound (1.4 g, 38% yield) as a brown solid. LC/MS (ESI, m/z): [(M+H)]⁺=556.2.

### Step 2—Methyl 3-((3-(cyclobutylcarbamoyl)-8-((4-methoxybenzyl)(methyl)amino)imidazo[1,2-b]pyridazin-6-yl)amino)-2-oxo-2H-[1,2′-bipyridine]-5′-carboxylate

To a stirred solution of 6-{[5′-(methoxycarbonyl)-2-oxo-[1,2′-bipyridin]-3-yl]amino}-8-{[(4-methoxyphenyl)methyl](methyl)amino}imidazo[1,2-b]pyridazine-3-carboxylic acid (300 mg, 0.5 mmol) and cyclobutylamine (38.41 mg, 0.540 mmol) in DMA (6 mL) were added HATU (246.39 mg, 0.648 mmol) and TEA (0.23 mL, 1.6 mmol) at rt under nitrogen atmosphere. The resulting mixture was stirred for 2 h at rt under nitrogen atmosphere. On completion, the reaction mixture was poured into water (30 mL). The precipitated solid was collected by filtration and washed with CH₂Cl₂ (5×5 mL), then dried under vacuum to afford the title compound (300 mg, 91% yield) as a brown solid. LC/MS (ESI, m/z): [(M+H)]⁺=609.2.

### Step 3—3-((3-(Cyclobutylcarbamoyl)-8-((4-methoxybenzyl)(methyl)amino)imidazo[1,2-b]pyridazin-6-yl)amino)-2-oxo-2H-[1,2′-bipyridine]-5′-carboxylic Acid

To a solution of methyl 3-{[3-(cyclobutylcarbamoyl)-8-{[(4-methoxyphenyl)methyl](methyl)amino}imidazo[1,2-b]pyridazin-6-yl]amino}-2-oxo-[1,2′-bipyridine]-5′-carboxylate (300 mg, 0.7 mmol) in THF (10 mL) was added 2 N aq. NaOH (6 mL) dropwise at rt. The reaction mixture was stirred for 2 h at rt. On completion, the reaction mixture was concentrated under reduced pressure to remove THF. The remained aqueous phase was adjusted pH to 4 with 2 N aq. HCl. The precipitated solid was collected by filtration and washed with water (1×5 mL), then dried under vacuum to give the title compound (200 mg, 51% yield) as a brown solid. LC/MS (ESI, m/z): [(M+H)]⁺=595.3.

### Step 4—3-((3-(Cyclobutylcarbamoyl)-8-(methylamino)imidazo[1,2-b]pyridazin-6-yl)amino)-2-oxo-2H-[1,2′-bipyridine]-5′-carboxylic Acid

To a solution of 3-{[3-(cyclobutylcarbamoyl)-8-{[(4-methoxyphenyl)methyl](methyl)amino}imidazo[1,2-b]pyridazin-6-yl]amino}-2-oxo-[1,2′-bipyridine]-5′-carboxylic acid (108 mg, 0.182 mmol) in DCM (3 mL) was added TFA (2 mL) dropwise at rt. The reaction mixture was stirred for 2 h at rt under nitrogen atmosphere. On completion, the reaction mixture was concentrated under reduced pressure. The residue was triturated with Et₂O to give the title compound (80 mg) as a brown solid. LC/MS (ESI, m/z): [(M+H)]⁺=475.2.

### Example 1.105. Preparation of 3-((3-(cyclopropylcarbamoyl)-8-(methylamino)imidazo[1,2-b]pyridazin-6-yl)amino)-2-oxo-2H-[1,2′-bipyridine]-5′-carboxylic Acid (Intermediate DA)

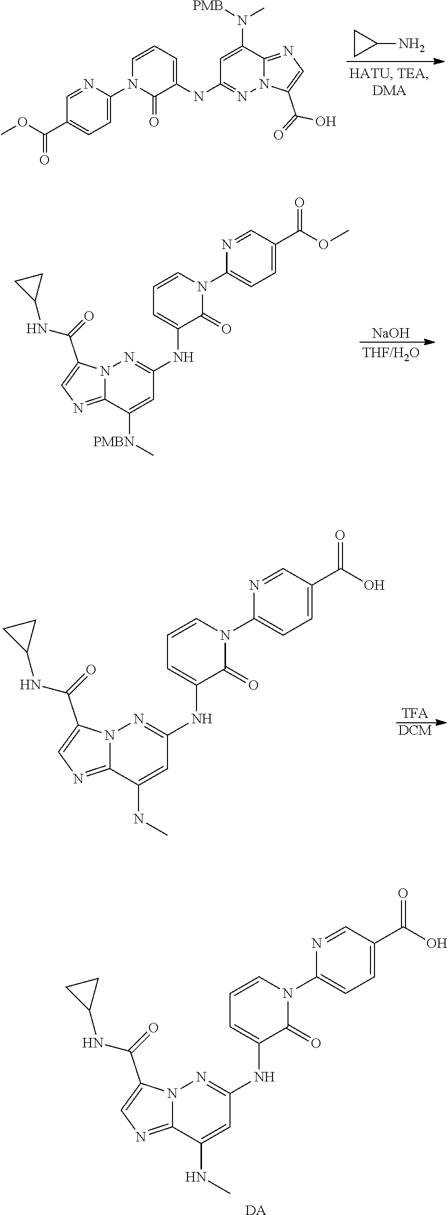

### Step 1—Methyl 3-((3-(cyclopropylcarbamoyl)-8-((4-methoxybenzyl)(methyl)amino)imidazo[1,2-b]pyridazin-6-yl)amino)-2-oxo-2H-[1,2′-bipyridine]-5′-carboxylate

To a stirred solution of 6-{[5′-(methoxycarbonyl)-2-oxo-[1,2′-bipyridin]-3-yl]amino}-8-{[(4-methoxyphenyl)methyl](methyl)amino}imidazo[1,2-b]pyridazine-3-carboxylic acid (400 mg, 0.7 mmol, synthesized via Step 1 of Intermediate CZ) and aminocyclopropane (41.11 mg, 0.720 mmol) in DMA (8 mL) were added HATU (382.53 mg, 0.864 mmol) and TEA (0.3 mL, 2 mmol) in turns at rt under nitrogen atmosphere. The resulting mixture was stirred for 2 h at rt under nitrogen atmosphere. On completion, the reaction mixture was poured into water (40 mL). The precipitated solid was collected by filtration and washed with CH₂Cl₂ (5×5 mL), then dried under vacuum to afford the title compound (400 mg, 93% yield) as a brown solid. LC/MS (ESI, m/z): [(M+H)]⁺=595.3.

### Step 2—3-((3-(Cyclopropylcarbamoyl)-8-((4-methoxybenzyl)(methyl)amino)imidazo[1,2-b]pyridazin-6-yl)amino)-2-oxo-2H-[1,2′-bipyridine]-5′-carboxylic Acid

To a solution of 3-{[3-(cyclopropylcarbamoyl)-8-{[(4-methoxyphenyl)methyl](methyl)amino}imidazo[1,2-b]pyridazin-6-yl]amino}-2-oxo-[1,2′-bipyridine]-5′-carboxylate (450 mg, 0.757 mmol) in THF (10 mL) was added 2 N aq. NaOH (6 mL) dropwise at rt. The reaction mixture was stirred for 2 h at rt. On completion, the reaction mixture was concentrated under reduced pressure to remove THF. The remained aqueous phase was adjusted pH to 4 with 2 N aq. HCl. The precipitated solid was collected by filtration and washed with water (1×5 mL), then dried under vacuum to give the title compound (200 mg, 46% yield) as a yellow solid. LC/MS (ESI, m/z): [(M+H)]⁺=581.3.

### Step 3—3-((3-(Cyclopropylcarbamoyl)-8-(methylamino)imidazo[1,2-b]pyridazin-6-yl)amino)-2-oxo-2H-[1,2′-bipyridine]-5′-carboxylic Acid

To a solution of 3-{[3-(cyclopropylcarbamoyl)-8-{[(4-methoxyphenyl)methyl](methyl)amino}imidazo[1,2-b]pyridazin-6-yl]amino}-2-oxo-[1,2′-bipyridine]-5′-carboxylic acid (200 mg, 0.3 mmol) in DCM (3 mL) was added TFA (2 mL) dropwise at rt. The reaction mixture was stirred for 2 h at rt under nitrogen atmosphere. On completion, the reaction mixture was concentrated under reduced pressure. The residue was triturated with Et₂O to give the title compound (100 mg) as a brown solid. LC/MS (ESI, m/z): [(M+H)]⁺=461.2.

### Example 1.106. Preparation of tert-butyl 4-((3-(4-(4,4,5,5-tetramethyl-1,3,2-dioxaborolan-2-yl)phenyl)cyclobutyl)methyl)piperazine-1-carboxylate and (4-(3-((4-(tert-butoxycarbonyl)piperazin-1-yl)methyl)cyclobutyl)phenyl)boronic Acid (Intermediate DB)

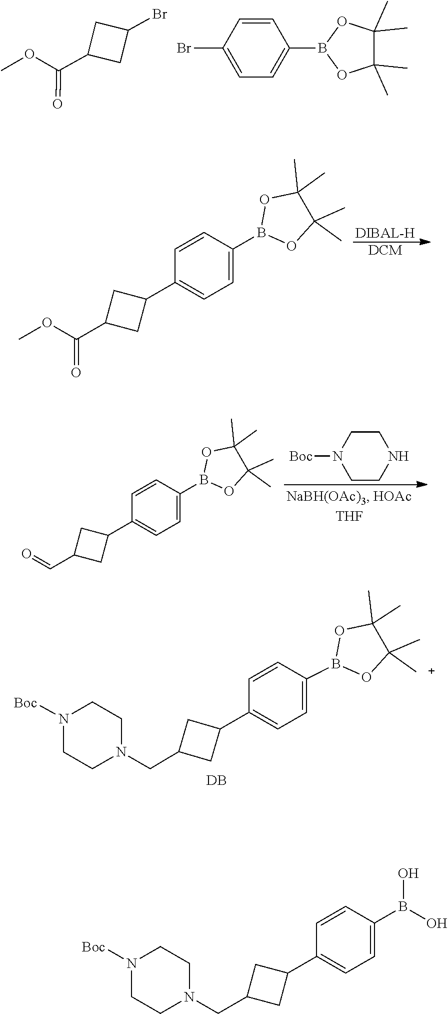

### Step 1—Methyl 3-(4-(4,4,5,5-tetramethyl-1,3,2-dioxaborolan-2-yl)phenyl)cyclobutane-1-carboxylate

To a solution of 2-(4-bromophenyl)-4,4,5,5-tetramethyl-1,3,2-dioxaborolane (15 g, 53 mmol), methyl 3-bromocyclobutanecarboxylate (13.3 g, 68.9 mmol), Ir[dF(CF₃)ppy]₂(dtbpy)(PF₆) (594 mg, 530 umol), NiCl₂.dtbbpy (105 mg, 265 umol), bis(trimethylsilyl)silyl-trimethyl-silane (13.1 g, 53.0 mmol), sodium carbonate (11.2 g, 106 mmol) in 1,2-dimethoxyethane (600 mL). The vial was sealed and placed under nitrogen was added. The reaction was stirred and irradiated with a 34 W blue LED lamp (7 cm away), with cooling fan to keep the reaction temperature at 25° C. for 14 hours. On completion, the reaction mixture was partitioned between water (100 mL) and ethyl acetate (300 mL). The organic phase was separated, dried over with anhydrous sodium sulfate, filtered and concentrated under reduced pressure to give a residue. The residue was purified by column chromatography (SiO₂, Petroleum ether/Ethyl acetate=40/1 to 5/1) to give the title compound (12 g, 54% yield) as a yellow oil. ¹H NMR (400 MHz, CDCl₃-d) δ=7.78 (dd, J=6.0, 8.0 Hz, 2H), 7.32-7.18 (m, 2H), 3.77 (s, 2H), 3.71 (s, 2H), 3.24-3.09 (m, 1H), 2.79-2.69 (m, 1H), 2.67-2.57 (m, 1H), 2.52-2.41 (m, 2H), 1.36 (s, 12H).

### Step 2—3-(4-(4,4,5,5-Tetramethyl-1,3,2-dioxaborolan-2-yl)phenyl)cyclobutane-1-carbaldehyde

To a solution of methyl 3-[4-(4,4,5,5-tetramethyl-1,3,2-dioxaborolan-2-yl)phenyl]cyclobutanecarboxylate (7 g, 20 mmol) in dichloromethane (120 mL) was added bis(2-methylpropyl)alumanylium; hydride (1 M, 26.5 mL) at −78° C. The mixture was stirred at −78° C. for 1 hour. On completion, the reaction mixture was quenched by addition with saturated ammonium chloride solution (70 mL) at −78° C., and then diluted with water (100 mL) and extracted with dichloromethane (250 mL×2). The combined organic layers were washed with brine (200 mL), dried over with anhydrous sodium sulfate, filtered and concentrated under reduced pressure to give a residue. The residue was purified by column chromatography (SiO₂, Petroleum ether/Ethyl acetate=40/1 to 1/1) to give the title compound (3.8 g, 59% yield) as a yellow oil. ¹H NMR (400 MHz, CDCl₃-d) δ=9.76 (d, J=1.6 Hz, 1H), 7.59 (dd, J=5.6, 8.0 Hz, 2H), 7.09-7.02 (m, 2H), 3.51-3.33 (m, 1H), 3.07-2.88 (m, 1H), 2.62-2.49 (m, 1H), 2.43-2.32 (m, 1H), 2.28-2.18 (m, 2H), 1.16 (s, 12H).

### Step 3—Tert-butyl 4-((3-(4-(4,4,5,5-tetramethyl-1,3,2-dioxaborolan-2-yl)phenyl)cyclobutyl)methyl)piperazine-1-carboxylate and (4-(3-((4-(tert-butoxycarbonyl)piperazin-1-yl)methyl)cyclobutyl)phenyl)boronic Acid

To a solution of 3-[4-(4,4,5,5-tetramethyl-1,3,2-dioxaborolan-2-yl)phenyl]cyclobutanecarbaldehyde (3.8 g, 13 mmol) and tert-butyl piperazine-1-carboxylate; hydrochloride (2.96 g, 13.2 mmol) in tetrahydrofuran (40 mL) was added acetic acid (3.99 g, 66.3 mmol) at 25° C. for 0.5 hour. Then sodium triacethoxy borohydride (5.63 g, 26.5 mmol) was added at 25° C. The mixture was stirred at 25° C. for 11.5 hours. On completion, the reaction mixture was quenched by addition of 1N sodium bicarbonate to pH=8 at 25° C., and then extracted with ethyl acetate (100 mL×3). The combined organic layers were washed with brine 150 mL, dried over with anhydrous sodium sulfate, filtered and concentrated under reduced pressure to give a residue. The crude product was purified by reversed-phase HPLC (0.1% FA condition) to give tert-butyl 4-((3-(4-(4,4,5,5-tetramethyl-1,3,2-dioxaborolan-2-yl)phenyl)cyclobutyl)methyl)piperazine-1-carboxylate (1.67 g, 14% yield) and (4-(3-((4-(tert-butoxycarbonyl)piperazin-1-yl)methyl)cyclobutyl)phenyl)boronic acid (1.67 g, 15% yield) as white solid. LC-MS (ESI+) m/z 457.3 (M+H)⁺. LC-MS (ESI+) m/z 375.3 (M+H)⁺.

### Example 1.107. Preparation of 3-(3-methyl-2-oxo-5-(4-(3-(piperazin-1-ylmethyl)cyclobutyl)phenyl)-2,3-dihydro-1H-benzo[d]imidazol-1-yl)piperidine-2,6-dione (Intermediate DC)

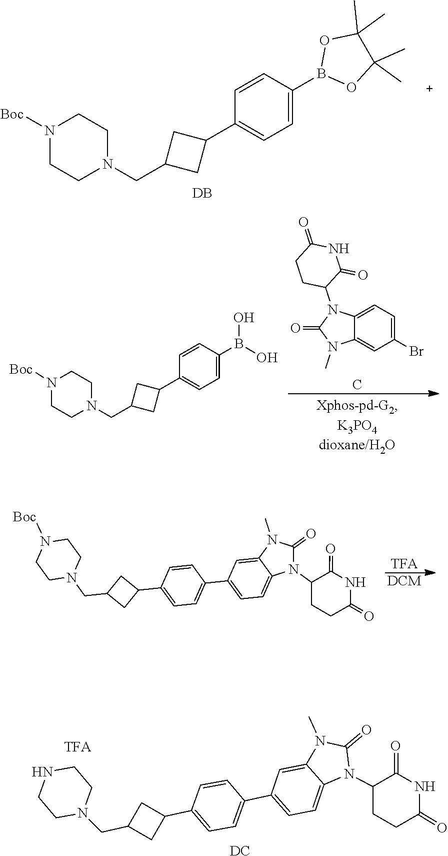

### Step 1—Tert-butyl 4-((3-(4-(1-(2,6-dioxopiperidin-3-yl)-3-methyl-2-oxo-2,3-dihydro-1H-benzo[d]imidazol-5-yl)phenyl)cyclobutyl)methyl)piperazine-1-carboxylate

To a solution of tert-butyl 4-[[3-[4-(4,4,5,5-tetramethyl-1,3,2-dioxaborolan-2-yl)phenyl]cyclobutyl]methyl]piperazine-1-carboxylate (1.67 g, 3.66 mmol) and [4-[3-[(4-tert-butoxycarbonylpiperazin-1-yl)methyl]cyclobutyl]phenyl]boronic acid (1.67 g, 4.46 mmol, Intermediate DB) and 3-(5-bromo-3-methyl-2-oxo-benzimidazol-1-yl)piperidine-2,6-dione (1.36 g, 4.02 mmol, Intermediate C) in dioxane (60 mL) and water (6 mL) was added chloro(2-dicyclohexylphosphino-2′,4′,6′-triisopropyl-1,1′-biphenyl)[2-(2′-amino-1,1′-biphenyl)]palladium(II) (287 mg, 365 umol) and potassium phosphate (1.55 g, 7.32 mmol). The mixture was stirred at 80° C. for 12 hours. On completion, the reaction mixture was partitioned between ethyl acetate (400 mL) and water (100 mL). The organic phase was separated, washed with brine (100 mL×2), dried over with anhydrous sodium sulfate, filtered and concentrated under reduced pressure to give a residue. The crude product was purified by reversed-phase HPLC (0.1% NH₄HCO₃) to give the title compound (1.43 g, 65% yield) as a white solid. ¹H NMR (400 MHz, DMSO-d6) δ=11.10 (s, 1H), 7.66-7.58 (m, 2H), 7.49-7.45 (m, 1H), 7.39-7.26 (m, 3H), 7.18 (d, J=8.2 Hz, 1H), 5.40 (dd, J=5.4, 12.8 Hz, 1H), 3.41 (s, 3H), 3.33-3.25 (m, 4H), 2.99-2.86 (m, 1H), 2.81-2.58 (m, 3H), 2.50-2.43 (m, 4H), 2.40-2.38 (m, 1H), 2.35-2.28 (m, 4H), 2.25-2.10 (m, 1H), 2.09-2.00 (m, 1H), 1.78-1.72 (m, 1H), 1.40 (s, 9H); LC-MS (ESI+) m/z 588.1 (M+H)⁺.

### Step 2—3-(3-Methyl-2-oxo-5-(4-(3-(piperazin-1-ylmethyl)cyclobutyl)phenyl)-2,3-dihydro-1H-benzo[d]imidazol-1-yl)piperidine-2,6-dione Trifluoroacetate

To a solution of tert-butyl 4-[(3-{4-[1-(2,6-dioxopiperidin-3-yl)-3-methyl-2-oxo-1,3-benzodiazol-5-yl]phenyl}cyclobutyl)methyl]piperazine-1-carboxylate (50 mg, 0.085 mmol) in DCM (3 mL) was added TFA (1 mL) dropwise at rt. The reaction mixture was stirred for 2 h at rt under nitrogen atmosphere. On completion, the reaction mixture was concentrated under reduced pressure. The residue was triturated with Et₂O to afford the title compound (35 mg) as a white solid. LC/MS (ESI, m/z): [(M+H)]⁺=488.3.

### Example 1.108. Preparation of 3-(3-methyl-2-oxo-5-(6-(2-oxopiperazin-1-yl)pyridin-3-yl)-2,3-dihydro-1H-benzo[d]imidazol-1-yl)piperidine-2,6-dione (Intermediate DD)

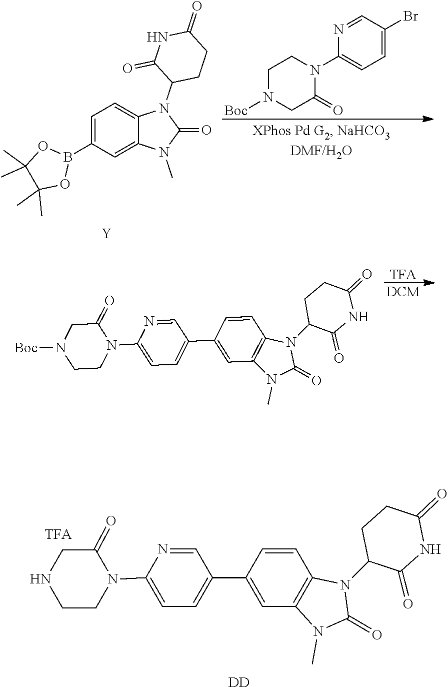

### Step 1—Tert-butyl 4-(5-(1-(2,6-dioxopiperidin-3-yl)-3-methyl-2-oxo-2,3-dihydro-1H-benzo[d]imidazol-5-yl)pyridin-2-yl)-3-oxopiperazine-1-carboxylate

A mixture of tert-butyl 4-(5-bromopyridin-2-yl)-3-oxopiperazine-1-carboxylate (1 g, 2.81 mmol, synthesized via Step 1 of Intermediate BQ), 3-(3-methyl-2-oxo-5-(4,4,5,5-tetramethyl-1,3,2-dioxaborolan-2-yl)-2,3-dihydro-1H-benzo[d]imidazol-1-yl)piperidine-2,6-dione (1.08 g, 2.81 mmol, Intermediate Y), XPhos Pd G2 (221 mg, 281 umol), and NaHCO₃ (471 mg, 5.61 mmol, 218 uL) in H₂O (1 mL) and DMF (10 mL) was degassed and purged with N₂ for three times. Then the mixture was stirred at 80° C. for 4 h under N₂ atmosphere. On completion, the reaction mixture was quenched by addition of H₂O (20 mL) at 20° C., then extracted with EtOAc (20 mL×3). The combined organic layers were washed with brine (20 mL×2), dried over Na₂SO₄, filtered and concentrated under reduced pressure to give a residue. This product was purified by prep-HPLC (0.1% FA condition) to give the title compound (600 mg, 40% yield) as yellow solid. ¹H NMR (400 MHz, DMSO-d₆) δ=11.14-11.09 (m, 1H), 8.80 (d, J=2.4 Hz, 1H), 8.16 (dd, J=2.4, 8.8 Hz, 1H), 7.96 (d, J=8.8 Hz, 1H), 7.61 (d, J=1.6 Hz, 1H), 7.45-7.38 (m, 1H), 7.26-7.20 (m, 1H), 5.42 (dd, J=5.2, 12.9 Hz, 1H), 4.20-4.05 (m, 4H), 3.72-3.63 (m, 2H), 3.42 (s, 3H), 2.99-2.86 (m, 1H), 2.81-2.62 (m, 2H), 2.09-2.00 (m, 1H), 1.48-1.43 (m, 9H)

### Step 2—3-(3-Methyl-2-oxo-5-(6-(2-oxopiperazin-1-yl)pyridin-3-yl)-2,3-dihydro-1H-benzo[d]imidazol-1-yl)piperidine-2,6-dione Trifluoroacetate

To a solution of tert-butyl 4-{5-[1-(2,6-dioxopiperidin-3-yl)-3-methyl-2-oxo-1,3-benzodiazol-5-yl]pyridin-2-yl}-3-oxopiperazine-1-carboxylate (150 mg, 0.282 mmol) in DCM (3 mL) was added TFA (1 mL) dropwise at rt. The reaction mixture was stirred for 6 h at rt, then concentrated under reduced pressure. The residue was triturated with Et₂O to afford the title compound (120 mg) as an off-white solid. LC/MS (ESI, m/z): [(M+H)]⁺=435.1.

### Example 1.109. Preparation of 3-(3-methyl-5-{4-[(3S)-3-{[methyl(piperidin-4-yl)amino]methyl}pyrrolidin-1-yl]phenyl}-2-oxo-1,3-benzodiazol-1-yl)piperidine-2,6-dione (Intermediate DE)

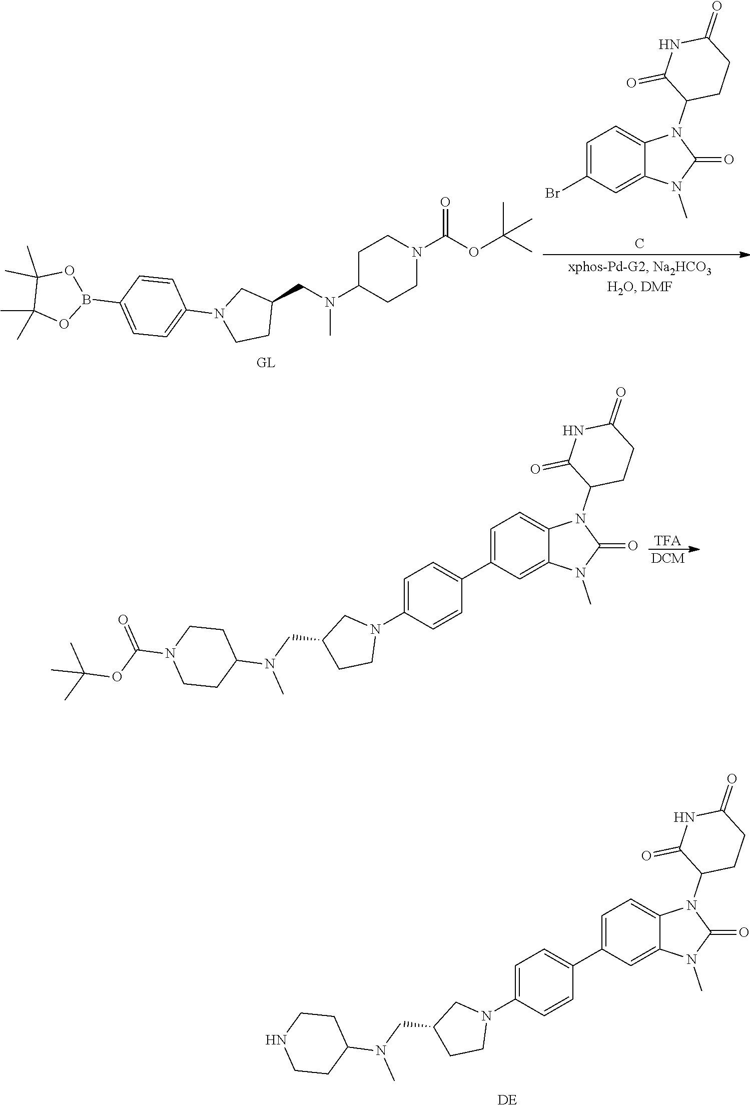

### Step 1—Tert-butyl 4-((((3R)-1-(4-(1-(2,6-dioxopiperidin-3-yl)-3-methyl-2-oxo-2,3-dihydro-1H-benzo[d]imidazol-5-yl)phenyl)pyrrolidin-3-yl)methyl)(methyl)amino)piperidine-1-carboxylate

A solution of (R)-tert-butyl 4-(methyl((1-(4-(4,4,5,5-tetramethyl-1,3,2-dioxaborolan-2-yl)phenyl)pyrrolidin-3-yl)methyl)amino)piperidine-1-carboxylate (900 mg, 2 mmol, Intermediate GL), 3-(5-bromo-3-methyl-2-oxo-benzimidazol-1-yl)piperidine-2,6-dione (609 mg, 1.80 mmol, Intermediate C), XPHOS-PD-G2 (142 mg, 180 umol) and NaHCO₃ (303 mg, 3.60 mmol) in H₂O (0.9 mL) and DMF (9 mL) was degassed and purged with N₂ three times. Then the mixture was stirred at 100° C. for 12 h under N₂ atmosphere. On completion, the reaction mixture was quenched with NH₄Cl (10 mL), and then extracted with EtOAc (10 mL×3). The combined organic layers were washed with brine (10 mL×3), dried over Na₂SO₄, filtered and concentrated under reduced pressure to give a residue. The residue was purified by flash silica gel chromatography (ISCO®; 20 g SepaFlash® Silica Flash Column, Eluent of 100% Ethylacetate/Petroleum (0.1% TEA, 30% THF) ethergradient @ 80 mL/min) to give the title compound (370 mg, 26% yield) as a red solid. ¹H NMR (400 MHz, DMSO-d₆) δ=11.09 (br s, 1H), 7.50 (br d, J=8.0 Hz, 2H), 7.37 (s, 1H), 7.23 (br d, J=8.4 Hz, 1H), 7.11 (br d, J=8.0 Hz, 1H), 6.59 (br d, J=7.6 Hz, 2H), 5.37 (br dd, J=4.8, 12.4 Hz, 1H), 4.00-3.92 (m, 2H), 3.39 (br s, 3H), 3.24-3.23 (m, 2H), 3.00 (br dd, J=4.8, 8.8 Hz, 3H), 2.94-2.90 (m, 1H), 2.72-2.61 (m, 6H), 2.33 (br s, 2H), 2.22 (br s, 3H), 2.05 (br dd, J=5.6, 8.8 Hz, 2H), 1.72-1.59 (m, 4H), 1.38 (s, 9H); LC-MS (ESI+) m/z 631.3 (M+H)⁺.

### Step 2—3-(3-Methyl-5-{4-[(3S)-3-{[methyl(piperidin-4-yl)amino]methyl}pyrrolidin-1-yl]phenyl}-2-oxo-1,3-benzodiazol-1-yl)piperidine-2,6-dione Trifluoroacetate

To a solution of tert-butyl 4-({[(3S)-1-{4-[1-(2,6-dioxopiperidin-3-yl)-3-methyl-2-oxo-1,3-benzodiazol-5-yl]phenyl}pyrrolidin-3-yl]methyl}(methyl)amino)piperidine-1-carboxylate (50 mg, 0.079 mmol) in DCM (2.5 mL) was added TFA (0.5 mL) dropwise at rt. The reaction mixture was stirred for 1 h at rt under nitrogen atmosphere. On completion, the reaction mixture was concentrated under reduced pressure. The residue was triturated with Et₂O to afford the title compound (40 mg) as a yellow solid. LC/MS (ESI, m/z): [(M+H)]⁺=531.3.

### Example 1.110. Preparation of 3-{3-Methyl-2-oxo-5-[(3S)-3-(piperidin-4-yl)pyrrolidin-1-yl]-1,3-benzodiazol-1-yl}piperidine-2,6-dione (Intermediate DF)

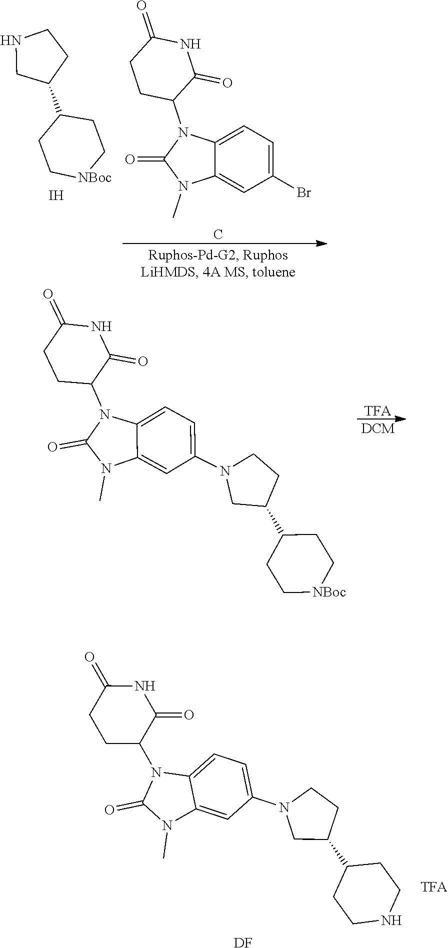

### Step 1—Tert-butyl 4-[(3R)-1-[1-(2,6-dioxo-3-piperidyl)-3-methyl-2-oxo-benzimidazol-5-yl]pyrrolidin-3-yl]piperidine-1-carboxylate

To a solution of 3-(5-bromo-3-methyl-2-oxo-benzimidazol-1-yl)piperidine-2,6-dione (886 mg, 2.62 mmol, Intermediate C), tert-butyl 4-[(3R)-pyrrolidin-3-yl]piperidine-1-carboxylate (800 mg, 3.15 mmol, Intermediate IH), dicyclohexyl(2′,6′-diisopropoxy-[1,1′-biphenyl]-2-yl)phosphane (2.45 g, 5.24 mmol), [2-(2-aminophenyl)phenyl]-chloro-palladium; dicyclohexyl-[2-(2,6-diisopropoxyphenyl)phenyl]phosphane (4.07 g, 5.24 mmol) and 4 Å molecular sieves (800 mg) in toluene (6 mL) was added lithium bis(trimethylsilyl)amide (1 M, 15.73 mL) in one portion. The reaction was degassed with nitrogen three times, then stirred at 25° C. for 0.16 hour. Then the mixture was heated to 100° C. for 2 hours under nitrogen. The reaction mixture was quenched with saturated ammonium chloride solution (100 mL) at 0° C., and extracted with ethyl acetate (100 mL×3). The filtrate dried over sodium anhydrous sodium sulfate, and concentrated under reduced pressure to give a residue. The residue was purified by perp-HPLC (column: Phenomenex Synergi Polar-RP 100*25 mm*4 um; mobile phase: [water (TFA)-ACN]; B %: 38%-58%, 7 min) to give the title compound (116 mg, 21% yield) as a white solid. ¹H NMR (400 MHz, DMSO-d₆) δ=11.04 (s, 1H), 6.90 (d, J=8.4 Hz, 1H), 6.42 (s, 1H), 6.24 (d, J=7.2 Hz, 1H), 5.28-5.23 (m, 1H), 3.96 (m, 2H), 3.29 (s, 3H), 3.26-3.18 (m, 2H), 2.91 (d, J=15.6 Hz, 2H), 2.73-2.61 (m, 4H), 2.13-1.96 (m, 4H), 1.74-1.60 (m, 4H), 1.39 (s, 9H), 1.12 (m, 2H); LC-MS (ESI+) m/z 512.3 (M+H)⁺.

### Step 2—3-{3-Methyl-2-oxo-5-[(3S)-3-(piperidin-4-yl)pyrrolidin-1-yl]-1,3-benzodiazol-1-yl}piperidine-2,6-dione Trifluoroacetate

To a solution of tert-butyl 4-[(3S)-1-[1-(2,6-dioxopiperidin-3-yl)-3-methyl-2-oxo-1,3-benzodiazol-5-yl]pyrrolidin-3-yl]piperidine-1-carboxylate (50 mg, 0.098 mmol) in DCM (2.5 mL) was added TFA (0.5 mL) dropwise at rt. The reaction mixture was stirred for 1 h at rt under nitrogen atmosphere. On completion, the reaction mixture was concentrated under reduced pressure. The residue was triturated with Et₂O to afford the title compound (40 mg) as a yellow solid. LC/MS (ESI, m/z): [(M+H)]⁺=412.2.

### Example 1.111. Preparation of 3-{3-methyl-2-oxo-5-[(3R)-3-(piperidin-4-yl)pyrrolidin-1-yl]-1,3-benzodiazol-1-yl}piperidine-2,6-dione (Intermediate DG)

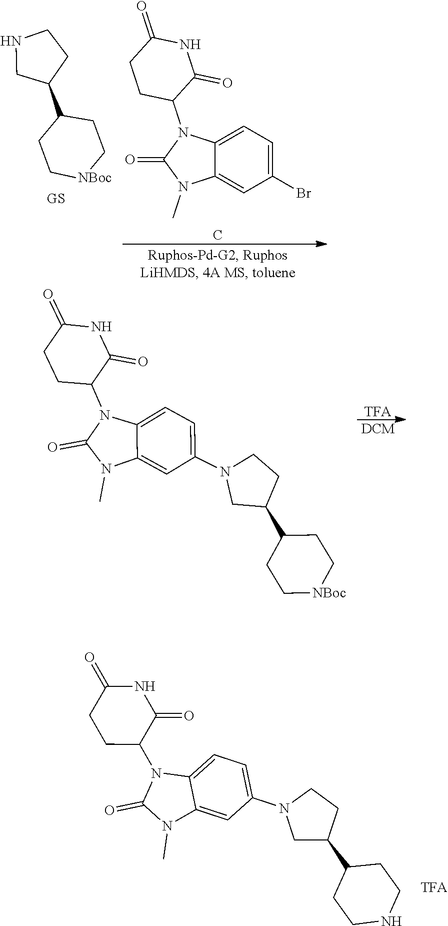

### Step 1—tert-butyl 4-[(3S)-1-[1-(2,6-dioxo-3-piperidyl)-3-methyl-2-oxo-benzimidazol-5-yl]pyrrolidin-3-yl]piperidine-1-carboxylate

To a solution of 3-(5-bromo-3-methyl-2-oxo-benzimidazol-1-yl)piperidine-2,6-dione (886 mg, 2.62 mmol, Intermediate C), tert-butyl 4-[(3S)-pyrrolidin-3-yl]piperidine-1-carboxylate (800 mg, 3.15 mmol, Intermediate GS), dicyclohexyl(2′,6′-diisopropoxy-[1,1′-biphenyl]-2-yl)phosphane (244 mg, 524 umol), [2-(2-aminophenyl)phenyl]-chloro-palladium; dicyclohexyl-[2-(2,6-diisopropoxyphenyl)phenyl]phosphane (407 mg, 524 umol) and 4 Å molecular sieves (800 mg) in toluene (25 mL) was added lithium bis(trimethylsilyl)amide (1 M, 15.7 mL) in one portion. Then the reaction was degassed with nitrogen three times, then stirred at 25° C. for 0.16 hr. Then the reaction mixture was heated to 100° C. and stirred for 2 hrs under nitrogen. The reaction mixture was quenched with saturated ammonium chloride solution (100 mL) at 0° C., and extracted with ethyl acetate (100 mL×3). The combined organic layers were dried over anhydrous sodium sulfate, concentrated under reduced pressure to give a residue. The residue was purified by perp-HPLC (column: Phenomenex luna C18 150*40 mm*15 um; mobile phase: [water (FA)-ACN]; B %: 40%-70%, 10 min) to give the title compound (239.2 mg, 16% yield) as a white solid. ¹H NMR (400 MHz, DMSO-d₆) δ=11.03 (s, 1H), 6.88 (d, J=8.8 Hz, 1H), 6.38 (d, J=2.0 Hz, 1H), 6.21-6.19 (m, 1H), 5.27-5.22 (m, 1H), 4.04-3.88 (m, 2H), 3.28 (s, 3H), 3.26-3.17 (m, 2H), 2.95-2.85 (m, 2H), 2.74-2.57 (m, 4H), 2.18-1.92 (m, 4H), 1.77-1.56 (m, 4H), 1.39 (s, 9H), 1.14-1.06 (m, 2H); LC-MS (ESI+) m/z 512.3 (M+H)⁺.

### Step 2—3-{3-Methyl-2-oxo-5-[(3R)-3-(piperidin-4-yl)pyrrolidin-1-yl]-1,3-benzodiazol-1-yl}piperidine-2,6-dione Trifluoroacetate

To a solution of tert-butyl 4-[(3R)-1-[1-(2,6-dioxopiperidin-3-yl)-3-methyl-2-oxo-1,3-benzodiazol-5-yl]pyrrolidin-3-yl]piperidine-1-carboxylate (50 mg, 0.1 mmol) in DCM (2.5 mL) was added TFA (0.5 mL) dropwise at rt. The reaction mixture was stirred for 1 h at rt under nitrogen atmosphere. On completion, the reaction mixture was concentrated under reduced pressure. The residue was triturated with Et₂O to afford the title compound (40 mg) as a yellow solid. LC/MS (ESI, m/z): [(M+H)]⁺=412.2.

### Example 1.112. Preparation of 3-Amino-[1,2′-bipyridin]-2-one (Intermediate DH)

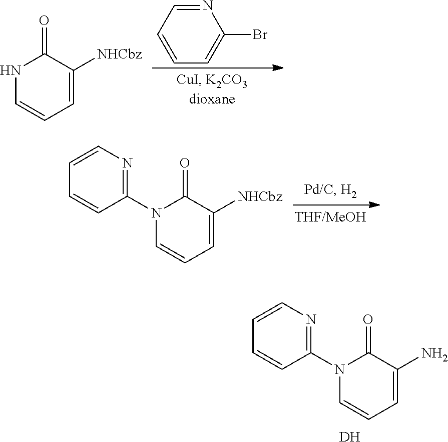

### Step 1—Benzyl N-{2-oxo-[1,2′-bipyridin]-3-yl}carbamate

To a stirred mixture of benzyl N-(2-oxo-1H-pyridin-3-yl)carbamate (20 g, 80 mmol, CAS #147269-67-8) and 2-bromopyridine (19.41 g, 122.8 mmol) in 1,4-dioxane (200 mL) were added DMEDA (0.72 g, 8.2 mmol), CuI (1.56 g, 8.188 mmol), and K₂CO₃ (22.63 g, 163.766 mmol) in portions at rt under air atmosphere. The resulting mixture was stirred for additional 16 h at reflux. On completion, the reaction mixture was cooled to rt and concentrated under vacuum. The residue was purified by trituration with H₂O (500 mL). The precipitated solids were collected by filtration and dried under vacuum to afford the title compound (12 g, 46% yield) as a green solid. LC/MS (ESI, m/z): [(M+H)]⁺=322.1.

### Step 2—3-Amino-[1,2′-bipyridin]-2-one

To a stirred solution of benzyl N-{2-oxo-[1,2′-bipyridin]-3-yl}carbamate (23 g, 72 mmol) in THF (200 mL) was added Pd/C (0.76 g, 7.2 mmol, 10 wt %) in portions at rt under nitrogen atmosphere. The resulting mixture was stirred for 4 h at rt under hydrogen atmosphere. The resulting mixture was filtered, and the filter cake was washed with THF (4×10 mL). The filtrate was concentrated under reduced pressure to afford the title compound (8 g, 60% yield) as an off-white solid. LC/MS (ESI, m/z): [(M+H)]⁺=188.1.

### Example 1.113. Preparation of methyl 6-chloro-8-methoxyimidazo[1,2-b]pyridazine-3-carboxylate (Intermediate DI)

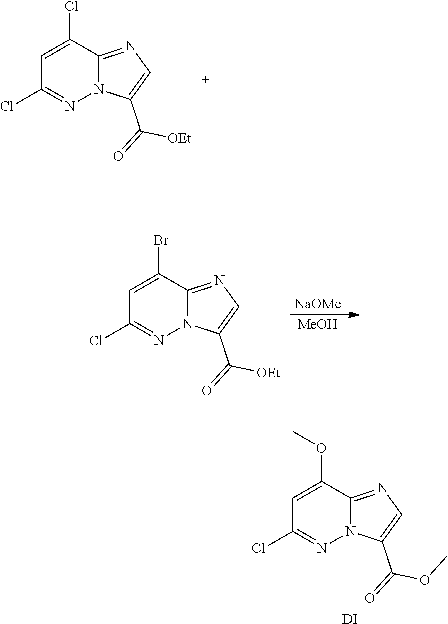

To a stirred solution of (mixture of ethyl 6,8-dichloroimidazo[1,2-b]pyridazine-3-carboxylate and ethyl 8-bromo-6-chloroimidazo[1,2-b]pyridazine-3-carboxylate (20 g, 70 mmol, synthesized via Step 1 of Intermediate F) in MeOH (250 mL) was added 30 wt % NaOMe in MeOH (36.56 mL, 198.0 mmol) dropwise at rt under nitrogen atmosphere. The resulting mixture was stirred for 5 h at rt under nitrogen atmosphere. After completion of the reaction, the reaction mixture was concentrated under reduced pressure. The residue was triturated with Et₂O (400 mL) and stirred for 1 h. The mixture was then filtered and the filter cake was washed with 3×100 mL of diethyl ether and dried in vacuo to afford the title compound (15 g, 76% yield) as a yellow solid. LC/MS (ESI, m/z): [(M+H)]⁺=242.1.

### Example 1.114. Preparation of 3-[5-(7-aminoheptyl)-3-methyl-2-oxo-1,3-benzodiazol-1-yl]piperidine-2,6-dione (Intermediate DJ)

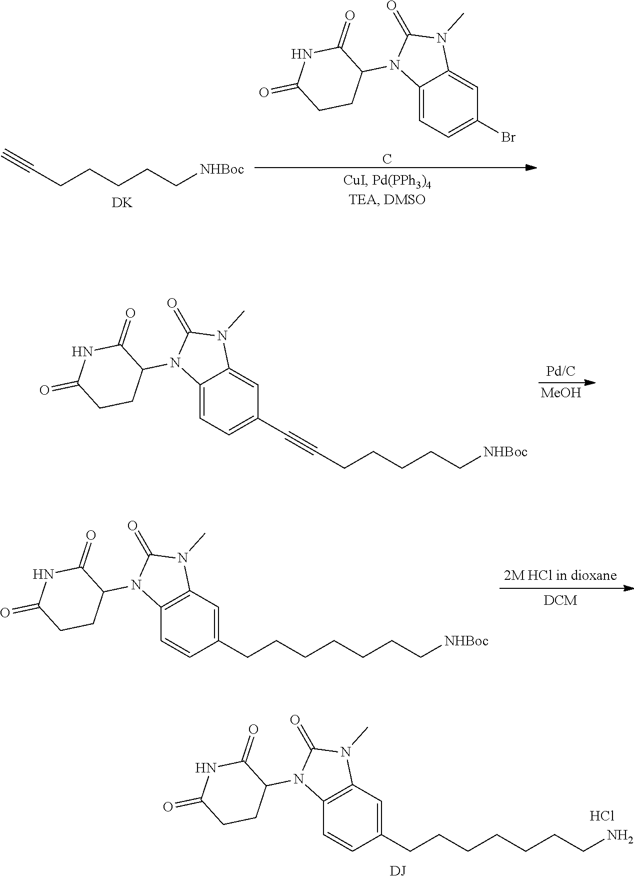

### Step 1—Tert-butyl N-[7-[1-(2,6-dioxopiperidin-3-yl)-3-methyl-2-oxo-1,3-benzodiazol-5-yl]hept-6-yn-1-yl]carbamate

To a stirred mixture of 3-(5-bromo-3-methyl-2-oxo-1,3-benzodiazol-1-yl)piperidine-2,6-dione (8.00 g, 23.7 mmol, Intermediate C), tert-butyl N-(hept-6-yn-1-yl)carbamate (10.00 g, 47.31 mmol, Intermediate DK) and CuI (0.45 g, 2.4 mmol) in DMSO (40.00 mL)/TEA (20.00 mL) was added Pd(PPh₃)₄ (2.73 g, 2.37 mmol) at rt under nitrogen atmosphere. The mixture was allowed to react for 4 h at 85° C. Upon completion, the mixture was cooled to rt and the reaction was diluted with water (100 mL). The resulting mixture was extracted with EtOAc (3×100 mL). The combined organic layers were washed with brine (2×100 mL), and dried over anhydrous Na₂SO₄. After filtration, the filtrate was concentrated under reduced pressure. The residue was purified by silica gel column chromatography, eluted with PE/EtOAc (1:2), then further purified by reverse phase flash chromatography (Column: WelFlash™ C18-I, 20-40 nm, 330 g; Eluent A: Water (plus 10 mmol/L FA); Eluent B: ACN; Gradient: 40%-65% B in 20 min; Flow rate: 80 mL/min; Detector: 220/254 nm; desired fractions were collected at 60% B) and concentrated under reduced pressure to afford the title compound (7.5 g, 68% yield) as a white solid. ¹H NMR (400 MHz, DMSO-d₆) δ 11.13 (s, 1H), 7.25-7.23 (m, 1H), 7.11-7.07 (m, 2H), 6.82 (t, J=5.8 Hz, 1H), 5.38 (dd, J=12.7, 5.3 Hz, 1H), 3.35 (s, 3H), 2.95-2.81 (m, 3H), 2.79-2.56 (m, 2H), 2.41 (t, J=7.0 Hz, 2H), 2.05-1.99 (m, 1H), 1.54 (t, J=7.0 Hz, 2H), 1.41-1.37 (m, 13H). LC/MS (ESI, m/z): [(M+1)]⁺=469.3.

### Step 2—Tert-butyl N-[7-[1-(2,6-dioxopiperidin-3-yl)-3-methyl-2-oxo-1,3-benzodiazol-5-yl]heptyl]carbamate

To a stirred solution of tert-butyl N-[7-[1-(2,6-dioxopiperidin-3-yl)-3-methyl-2-oxo-1,3-benzodiazol-5-yl]hept-6-yn-1-yl]carbamate (8.00 g, 17.1 mmol) in MeOH (200.00 mL)/AcOH (10.00 mL) was added Pd/C (1.00 g, 9.397 mmol) at rt under nitrogen atmosphere. The reaction was purged with H₂ three times and stirred for 16 h at rt under hydrogen atmosphere. On completion, the reaction mixture was filtered, and the filter cake was washed with CH₂Cl₂ (3×20 mL). The filtrate was concentrated under reduced pressure to give the title compound (7.5 g, 93% yield) as a white solid. ¹H NMR (400 MHz, DMSO-d₆) δ 11.09 (s, 1H), 7.05-6.96 (m, 2H), 6.88-6.84 (m, 1H), 6.76 (t, J=5.8 Hz, 1H), 5.34 (dd, J=12.7, 5.3 Hz, 1H), 3.35 (s, 3H), 2.91-2.86 (m, 3H), 2.78-2.55 (m, 4H), 2.03-1.98 (m, 1H), 1.58 (t, J=7.4 Hz, 2H), 1.37 (s, 9H), 1.32-1.17 (m, 6H). LC/MS (ESI, m/z): [(M+1)]⁺=473.3.

### Step 3—3-[5-(7-Aminoheptyl)-3-methyl-2-oxo-1,3-benzodiazol-1-yl]piperidine-2,6-dione Hydrochloride

To a stirred solution of tert-butyl N-[7-[1-(2,6-dioxopiperidin-3-yl)-3-methyl-2-oxo-1,3-benzodiazol-5-yl]heptyl]carbamate (7.50 g, 15.9 mmol) in DCM (20.00 mL) was added HCl (4M) in 1,4-dioxane (20.00 mL) at rt under nitrogen atmosphere. The mixture was stirred for 4 h at rt. On completion, the mixture was concentrated under reduced pressure. The residue was triturated with Et₂O to afford the title compound (6.3 g, 97% yield) as an off-white solid. ¹H NMR (300 MHz, DMSO-d₆) δ 11.09 (s, 1H), 7.92 (broad, 3H), 7.07-6.96 (m, 2H), 6.88-6.84 (m, 1H), 5.35 (dd, J=12.8, 5.3 Hz, 1H), 3.32 (s, 3H) 3.02-2.82 (m, 1H), 2.78-2.71 (m, 3H), 2.68-2.56 (m, 3H), 2.06-1.95 (m, 1H), 1.62-1.51 (m, 4H), 1.32-1.27 (m, 6H). LC/MS (ESI, m/z): [(M+1)]⁺=373.3.

### Example 1.115. Preparation of tert-butyl N-(hept-6-yn-1-yl)carbamate (Intermediate DK)

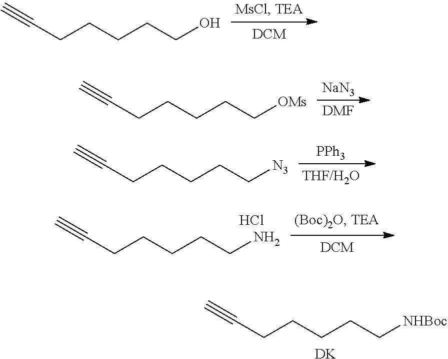

### Step 1—Hept-6-yn-1-yl methanesulfonate

To a stirred solution of hept-6-yn-1-ol (30.00 g, 267.4 mmol) in DCM (450.00 mL) were added TEA (92.68 mL, 668.6 mmol) and MsCl (45.95 g, 401.2 mmol) in portions at 0° C. under nitrogen atmosphere. The resulting mixture was stirred for 2 h at rt under nitrogen atmosphere. The resulting mixture was diluted with water (250 mL). The resulting mixture was extracted with CH₂Cl₂ (3×100 mL). The combined organic layers were washed with brine (1×100 mL), and dried over anhydrous Na₂SO₄. After filtration, the filtrate was concentrated under reduced pressure to give the title compound (50 g). ¹H NMR (400 MHz, CDCl₃) δ 4.28-4.24 (m, 2H), 3.03 (s, 3H), 2.27-2.22 (m, 2H), 1.98 (s, 1H), 1.84-1.77 (m, 2H), 1.60-1.52 (m, 6H).

### Step 2—7-Azidohept-1-yne

To a stirred solution of hept-6-yn-1-yl methanesulfonate (50.00 g, 262.8 mmol) in DMF (500.00 mL) was added NaN₃ (25.63 g, 394.2 mmol) at rt under nitrogen atmosphere. The resulting mixture was stirred overnight at 50° C. under nitrogen atmosphere. The resulting mixture was diluted with water (1 L). The resulting mixture was extracted with EtOAc (3×200 mL). The combined organic layers were washed with brine (3×100 mL), and dried over anhydrous Na₂SO₄. After filtration, the filtrate was concentrated under reduced pressure to give the title compound (36 g) as light oil. ¹H NMR (400 MHz, Chloroform-d) δ 3.30 (t, J=6.8 Hz, 2H), 2.23 (td, J=6.8, 2.6 Hz, 2H), 1.97 (s, 1H), 1.72-1.41 (m, 6H).

### Step 3—Hept-6-yn-1-amine Hydrochloride

To a stirred solution of 7-azidohept-1-yne (36.00 g, 262.4 mmol) in THF (400 mL) and H₂O (40.00 mL) was added PPh₃ (103.24 g, 393.62 mmol) at 0° C. under nitrogen atmosphere. The resulting mixture was stirred for 2 h at 60° C. under nitrogen atmosphere. On completion, the solution was concentrated under reduced pressure and diluted with DCM (300 mL). The mixture was acidified to pH 1 with HCl (2 M aq.). The aqueous layer was extracted with DCM (3×100 mL). The aqueous layer was concentrated under reduced pressure to give afford to give the title compound (38 g, 98% yield) as a white solid. ¹H NMR (400 MHz, DMSO-d₆) δ 8.09 (broad, 3H), 2.82-2.67 (m, 3H), 2.18-2.14 (m, 2H), 1.61-1.53 (m, 2H), 1.49-1.35 (m, 4H). LC/MS (ESI, m/z): [(M+1)]⁺=112.2.

### Step 4—Tert-butyl N-(hept-6-yn-1-yl)carbamate

To a stirred solution of hept-6-yn-1-amine hydrochloride (28.00 g, 190.5 mmol) in DCM (500.00 mL) were added TEA (144.8 mL, 952.4 mmol) and Boc₂O (62.28 g, 285.7 mmol) in portions at rt under nitrogen atmosphere. The resulting mixture was stirred overnight at rt under nitrogen atmosphere. On completion, the reaction mixture was diluted with water (1 L). The resulting mixture was extracted with CH₂Cl₂ (3×200 mL). The combined organic layers were washed with brine (2×300 mL), dried over anhydrous Na₂SO₄. After filtration, the filtrate was concentrated under reduced pressure. The residue was purified by silica gel column chromatography, eluted with PE/EtOAc (1:1) to afford the title compound (40 g, 99% yield) as a light yellow solid. ¹H NMR (400 MHz, Chloroform-d) δ 4.56-4.52 (m, 1H), 3.16-3.12 (m, 2H), 2.21 (td, J=7.0, 2.6 Hz, 2H), 1.96 (t, J=2.7 Hz, 1H), 1.62-1.49 (m, 6H), 1.46 (s, 9H). LC/MS (ESI, m/z): [(M+1-56)]⁺=156.2.

### Example 1.116. Preparation of 3-(4-(7-aminoheptyl)-3-methyl-2-oxo-2,3-dihydro-1H-benzo[d]imidazol-1-yl)piperidine-2,6-dione (Intermediate DL)

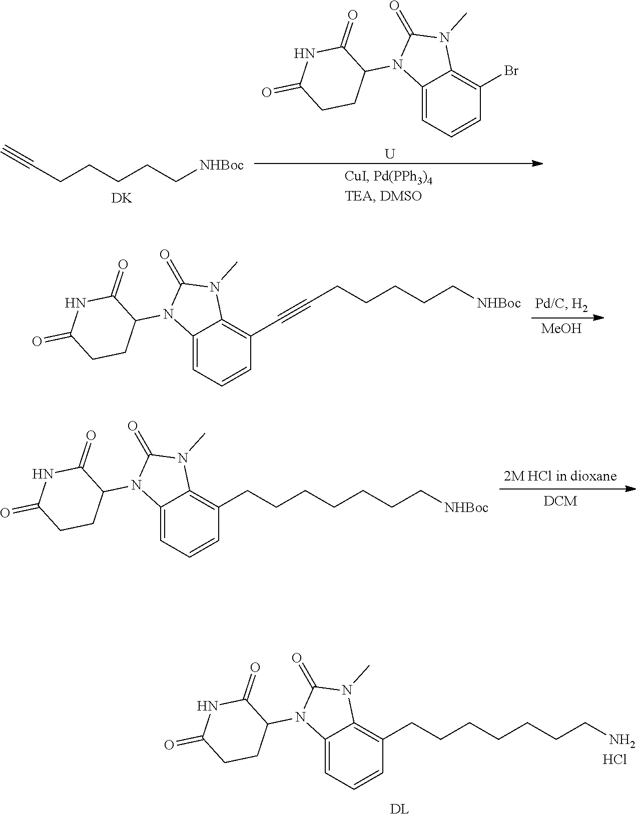

### Step 1—Tert-butyl N-[7-[1-(2,6-dioxopiperidin-3-yl)-3-methyl-2-oxo-1,3-benzodiazol-4-yl]hept-6-yn-1-yl]carbamate

To a stirred mixture of 3-(4-bromo-3-methyl-2-oxo-1,3-benzodiazol-1-yl)piperidine-2,6-dione (8.00 g, 23.7 mmol, Intermediate U) and tert-butyl N-(hept-6-yn-1-yl)carbamate (10.00 g, 47.32 mmol, Intermediate DK) in DMA (50.00 mL) and TEA (25.00 mL) were added CuI (450.55 mg, 2.366 mmol) and Pd(PPh₃)₄ (2.73 g, 2.37 mmol) at rt under nitrogen atmosphere. The resulting mixture was stirred for 3 h at 80° C. under nitrogen atmosphere. The resulting mixture was diluted with water (200 mL). The resulting mixture was extracted with EtOAc (3×100 mL). The combined organic layers were washed with brine (3×50 mL), and dried over anhydrous Na₂SO₄. After filtration, the filtrate was concentrated under reduced pressure. The residue product was purified by reverse phase flash (Column: Spherical C18, 20˜40 um, 330 g; Mobile Phase A: Water (0.05% FA), Mobile Phase B: ACN; Flow rate: 45 mL/min; Gradient (B %): 5%˜5%, 8 min; 40%˜70%, 30 min; 70%˜95%; 0 min; 95%, 5 min; Detector: 254 nm; Rt: 22.3 min.) to afford the title compound (6 g, 54% yield) as a light yellow solid. ¹H NMR (400 MHz, DMSO-d₆) δ 11.12 (s, 1H), 7.11 (d, J=7.7 Hz, 1H), 7.06 (d, J=7.6 Hz, 1H), 6.99 (t, J=7.8 Hz, 1H), 6.81 (t, J=5.9 Hz, 1H), 5.39 (dd, J=12.6, 5.4 Hz, 1H), 3.65 (s, 3H), 2.99-2.79 (m, 3H), 2.78-2.56 (m, 2H), 2.51-2.47 (m, 2H), 2.06-1.98 (m, 1H), 1.56 (d, J=6.9 Hz, 2H), 1.43-1.39 (m, 4H), 1.36 (s, 9H). LC/MS (ESI, m/z): [(M+1)]⁺=469.3.

### Step 2—Tert-butyl N-[7-[1-(2,6-dioxopiperidin-3-yl)-3-methyl-2-oxo-1,3-benzodiazol-4-yl]heptyl]carbamate

To a stirred solution tert-butyl N-[7-[1-(2,6-dioxopiperidin-3-yl)-3-methyl-2-oxo-1,3-benzodiazol-4-yl]hept-6-yn-1-yl]carbamate (6.50 g, 13.9 mmol) in MeOH (500.00 mL) was added Pd/C (2.21 g, 20.8 mmol) in portions at rt under nitrogen atmosphere. The resulting mixture was stirred overnight at rt under hydrogen atmosphere. The resulting mixture was filtered. The filter cake was washed with MeOH (5×100 mL). The filtrate was concentrated under reduced pressure to afford the title compound (6 g, 92% yield) as a white solid. ¹H NMR (400 MHz, DMSO-d₆) δ 11.12 (s, 1H), 6.97-6.93 (m, 2H), 6.88-6.85 (m, 1H), 6.77 (t, J=5.8 Hz, 1H), 5.37 (dd, J=12.6, 5.4 Hz, 1H), 3.55 (s, 3H), 2.95-2.86 (m, 5H), 2.80-2.57 (m, 2H), 2.04-1.96 (m, 1H), 1.563-1.56 (m, 2H), 1.41-1.22 (m, 17H). LC/MS (ESI, m/z): [(M+1)]⁺=473.3.

### Step 3—3-(4-(7-Aminoheptyl)-3-methyl-2-oxo-2,3-dihydro-1H-benzo[d]imidazol-1-yl)piperidine-2,6-dione Hydrochloride

To a stirred solution of tert-butyl N-[7-[1-(2,6-dioxopiperidin-3-yl)-3-methyl-2-oxo-1,3-benzodiazol-4-yl]heptyl]carbamate (6.00 g, 12.7 mmol) in DCM (50.00 mL) was added HCl (gas) in 1,4-dioxane (20 mL) dropwise at rt under air atmosphere. The resulting mixture was stirred for 2 h at rt under nitrogen atmosphere. The resulting mixture was concentrated under vacuum. The residue was triturated with Et₂O to afford the title compound (4.5 g, 90% yield) as a white solid. ¹H NMR (300 MHz, DMSO-d₆) δ 11.10 (s, 1H), 7.85 (broad, 3H), 7.01-6.92 (m, 2H), 6.88-6.84 (m, 1H), 5.38 (dd, J=12.7, 5.4 Hz, 1H), 3.56 (s, 3H), 2.97-2.83 (m, 3H), 2.80-2.63 (m, 4H), 2.05-1.91 (m, 1H), 1.64-1.51 (m, 4H), 1.43-1.24 (m, 6H). LC/MS (ESI, m/z): [(M+1)]⁺=373.3.

### Example 1.117. Preparation of tert-butyl N-methyl-N-[3-(prop-2-yn-1-yloxy)propyl]carbamate (Intermediate DM)

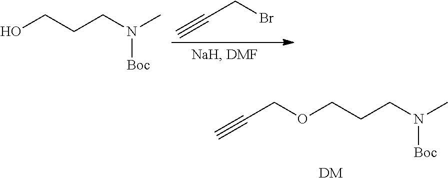

To a stirred solution of tert-butyl N-(3-hydroxypropyl)-N-methylcarbamate (25.00 g, 132.1 mmol, CAS #98642-44-5) in DMF (200.00 mL) was added NaH (4.76 g, 198 mmol) in portions at 0° C. under nitrogen atmosphere. The above mixture was stirred for 30 min at 0° C. under nitrogen atmosphere. To the above mixture was added propargyl bromide (23.57 g, 198.1 mmol) dropwise at 0° C. The resulting mixture was stirred for additional 16 h at rt. On completion, the reaction mixture was quenched with water (500 mL). The resulting mixture was extracted with EtOAc (3×300 mL). The combined organic layers were washed with brine (500 mL), and dried over anhydrous Na₂SO₄. After filtration, the filtrate was concentrated under reduced pressure. The resulting solution was purified on silica gel column chromatography, eluted with PE/EA (30:1). The resulting mixture was concentrated under vacuum to afford the title compound (47.7 g, 74% yield) as a yellow oil. ¹H NMR (400 MHz, Chloroform-d) δ 4.15 (d, J=2.4 Hz, 2H), 3.54 (t, J=6.3 Hz, 2H), 3.31 (t, J=7.0 Hz, 2H), 2.87 (s, 3H), 2.43 (t, J=2.4 Hz, 1H), 1.86-1.80 (m, 2H), 1.47 (s, 9H). LC/MS (ESI, m/z): [(M+1)]⁺=228.2.

### Example 1.118. Preparation of 3-(3-methyl-4-[3-[3-(methylamino)propoxy]propyl]-2-oxo-1,3-benzodiazol-1-yl)piperidine-2,6-dione (Intermediate DN)

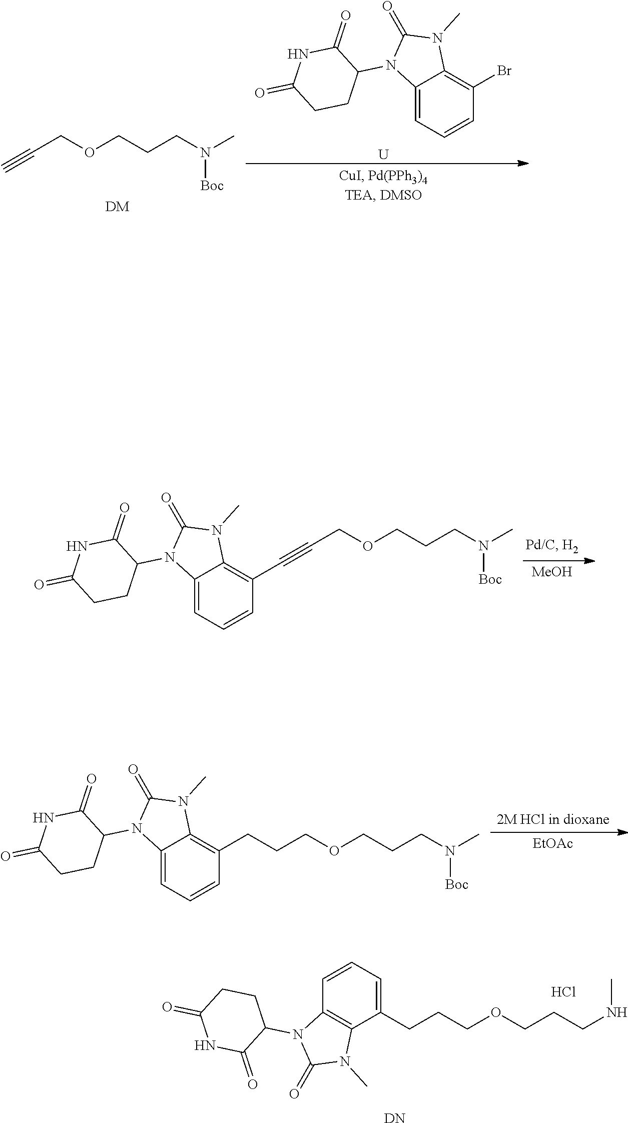

### Step 1—Tert-butyl N-(3-[2-[1-(2,6-dioxopiperidin-3-yl)-2-oxo-3H-1,3-benzodiazol-4-ylidyne]ethoxy]propyl)-N-methylcarbamate

To a stirred solution of tert-butyl N-methyl-N-[3-(prop-2-yn-1-yloxy)propyl]carbamate (16.50 g, 72.59 mmol, Intermediate DM) and 3-(4-bromo-3-methyl-2-oxo-1,3-benzodiazol-1-yl)piperidine-2,6-dione (4.91 g, 14.52 mmol) in DMSO (140 mL) were added Pd(PPh₃)₄ (8.39 g, 7.26 mmol) and CuI (392 mg, 7.26 mmol) and TEA (70 mL) dropwise at 25° C. under nitrogen atmosphere. The resulting mixture was stirred for 2 h at 80° C. under nitrogen atmosphere. On completion, the reaction mixture was cooled to rt and purified by reverse phase flash (Column: Spherical C18, 20˜40 um, 330 g; Mobile Phase A: Water (10 mmol/LAcOH), Mobile Phase B: ACN; Flow rate: 80 mL/min; Gradient (B %): 52%˜72%, 20 min; Detector: 254/220 nm); desired fractions were collected at 62% B) and concentrated under reduced pressure to afford the title compound (9.22 g, 80% yield) as a dark yellow foam. ¹H NMR (400 MHz, Chloroform-d) δ 8.20 (s, 1H), 7.19 (dd, J=7.9, 1.0 Hz, 1H), 7.01 (t, J=7.9 Hz, 1H), 6.78 (dd, J=8.0, 1.1 Hz, 1H), 5.22 (dd, J=12.5, 5.3 Hz, 1H), 4.42 (s, 2H), 3.79 (s, 3H), 3.62 (t, J=6.3 Hz, 2H), 3.34 (t, J=7.1 Hz, 2H), 3.03-2.92 (m, 1H), 2.89 (s, 3H), 2.87-2.68 (m, 2H), 2.33-2.20 (m, 1H), 1.92-1.81 (m, 2H), 1.47 (s, 9H). LC/MS (ESI, m/z): [(M+1)]⁺=485.3.

### Step 2—Tert-butyl N-(3-[3-[1-(2,6-dioxopiperidin-3-yl)-3-methyl-2-oxo-1,3-benzodiazol-4-yl]propoxy]propyl)-N-methylcarbamate

Pd/C was added in a mixture of tert-butyl N-[3-([3-[1-(2,6-dioxopiperidin-3-yl)-3-methyl-2-oxo-1,3-benzodiazol-4-yl]prop-2-yn-1-yl]oxy)propyl]-N-methylcarbamate (8.00 g) in THF (200.00 mL) at 25° C. The reaction mixture was purged with nitrogen three times and was stirred for 5 h at 25° C. under H₂ atmosphere. On completion, the reaction mixture was filtered. The filtrate was concentrated under reduced pressure to afford the title compound (7.96 g, 88% yield) as a white solid. ¹H NMR (400 MHz, Chloroform-d) δ 8.40 (s, 1H), 7.00 (t, J=7.8 Hz, 1H), 6.92 (d, J=7.7 Hz, 1H), 6.69 (d, J=7.8 Hz, 1H), 5.24 (dd, J=12.5, 5.3 Hz, 1H), 3.70 (s, 3H), 3.50-3.44 (m, 4H), 3.34-3.29 (m, 2H), 3.05-3.01 (m, 2H), 3.00-2.91 (m, 1H), 2.88 (s, 3H), 2.85-2.70 (m, 2H), 2.25-2.20 (m, 1H), 1.97-1.90 (m, 2H), 1.86-1.80 (m, 2H), 1.48 (s, 9H). LC/MS (ESI, m/z): [(M+1)]⁺=489.3.

### Step 3—3-(3-Methyl-4-[3-[3-(methylamino)propoxy]propyl]-2-oxo-1,3-benzodiazol-1-yl)piperidine-2,6-dione Hydrochloride

To a stirred mixture of tert-butyl N-(3-[3-[1-(2,6-dioxopiperidin-3-yl)-3-methyl-2-oxo-1,3-benzodiazol-4-yl]propoxy]propyl)-N-methylcarbamate (8.50 g) in EA (200.00 mL) were added HCl (gas) in 1,4-dioxane (100.00 mL) in portions at 25° C. under nitrogen atmosphere. The resulting mixture was stirred for 4 h at 25° C. under nitrogen atmosphere. On completion, the reaction mixture was concentrated under vacuum. The residue was purified by trituration with EA (100 mL) to give the title compound (7.34 g, 96% yield) as a white solid. ¹H NMR (400 MHz, DMSO-d₆) δ 11.10 (s, 1H), 8.89 (broad, 2H), 7.00-6.96 (m, 2H), 6.90-6.86 (m, 1H), 5.39 (dd, J=12.6, 5.4 Hz, 1H), 3.57 (s, 3H), 3.49-3.43 (m, 5H), 2.98-2.86 (m, 5H), 2.77-2.57 (m, 2H), 2.52-2.48 (m, 2H), 2.05-1.95 (m, 1H), 1.94-1.80 (m, 4H). LC/MS (ESI, m/z): [(M+1)]⁺=389.3.

### Example 1.119. Preparation of tert-butyl (2-(2-(2-bromoethoxy)ethoxy)ethyl)carbamate (Intermediate DO)

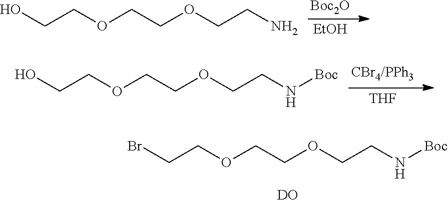

### Step 1—Tert-butyl (2-(2-(2-hydroxyethoxy)ethoxy)ethyl)carbamate

To a solution of 2-(2-(2-aminoethoxy)ethoxy)ethanol (24.5 g, 164 mmol, CAS #929-06-6) in EtOH (250 mL) was added a solution of (Boc)₂O (43.01 g, 197.1 mmol, 45.27 mL) in EtOH (100 mL) at 0° C. The mixture was stirred at 25° C. for 16 h. On completion, the mixture was concentrated in vacuo to afford the title compound (50 g) as colorless oil.

### Step 2—Tert-butyl (2-(2-(2-bromoethoxy)ethoxy)ethyl)carbamate

To a solution of tert-butyl (2-(2-(2-hydroxyethoxy)ethoxy)ethyl)carbamate (35 g, 140 mmol) and PPh₃ (110.47 g, 421.17 mmol) in THF (500 mL) was added CBr₄ (139.67 g, 421.17 mmol) slowly at 0° C. The resulting mixture was stirred at 20° C. for 3 h. On completion, the mixture was filtered and the filter cake was washed with EA (50 mL). The combined filtrate was concentrated in vacuo to afford a residue. The residue was purified by column chromatography (SiO₂, PE:EA=10:1˜1:1) to afford the title compound (43 g, 98% yield) as colorless oil. ¹H NMR (400 MHz, CHLOROFORM-d) δ=5.01 (br s, 1H), 3.83 (t, J=6.4 Hz, 2H), 3.73-3.46 (m, 8H), 3.34 (br d, J=5.0 Hz, 2H), 1.46 (s, 9H).

### Example 1.120. Preparation of 3-(5-{2-[2-(2-aminoethoxy)ethoxy]ethyl}-3-methyl-2-oxo-1,3-benzodiazol-1-yl)piperidine-2,6-dione (Intermediate DP)

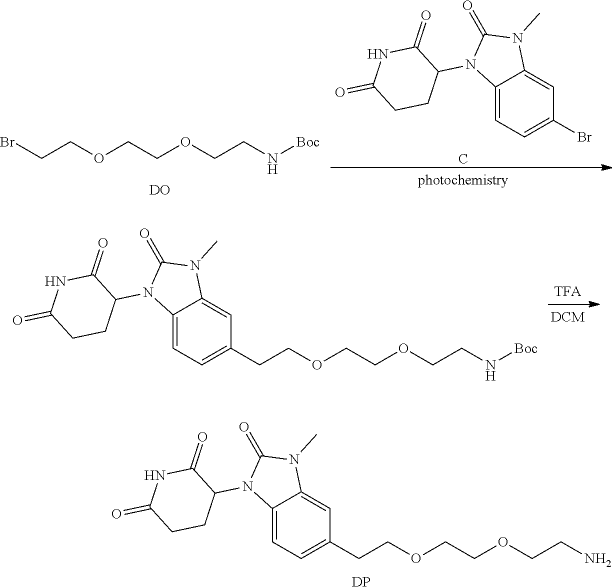

### Step 1—Tert-butyl (2-(2-(2-(1-(2,6-dioxopiperidin-3-yl)-3-methyl-2-oxo-2,3-dihydro-1H-benzo[d]imidazol-5-yl)ethoxy)ethoxy)ethyl)carbamate

To a solution of 3-(5-bromo-3-methyl-2-oxo-2,3-dihydro-1H-benzo[d]imidazol-1-yl)piperidine-2,6-dione (5 g, 15 mmol, Intermediate C) and tert-butyl (2-(2-(2-bromoethoxy) ethoxy)ethyl)carbamate (6.00 g, 19.2 mmol, Intermediate DO) in DME (50 mL) was added Na₂CO₃ (3.13 g, 29.6 mmol), Ir[dF(CF₃)ppy]₂(dtbpy)(PF₆) (165.89 mg, 147.86 umol) and TTMSS (3.68 g, 14.79 mmol, 4.56 mL) at 10° C. The reaction was stirred at 25° C. for 16 hr irradiated with a 34 W blue LED lamp (7 cm away). On completion, the mixture was filtered and filter cake was washed with EA (20 mL). The filtrate was concentrated in vacuo to afford a residue. The residue was purified by column chromatography (SiO₂, PE:EA=3:1˜1:1) to afford a residue. The residue was further purified by reversed-phase HPLC (0.1% FA condition) to afford the title compound (3.57 g, 49% yield) as colorless oil. ¹H NMR (400 MHz, CHLOROFORM-d) δ=6.99-6.89 (m, 2H), 6.75 (d, J=7.8 Hz, 1H), 5.23 (dd, J=5.4, 12.7 Hz, 1H), 5.11-4.96 (m, 1H), 3.70 (t, J=7.0 Hz, 2H), 3.62 (s, 4H), 3.54 (br t, J=4.8 Hz, 2H), 3.44 (s, 3H), 3.32 (br d, J=4.8 Hz, 2H), 3.06-2.63 (m, 6H), 2.28-2.18 (m, 1H), 1.46 (s, 9H).

### Step 2—3-(5-{2-[2-(2-aminoethoxy)ethoxy]ethyl}-3-methyl-2-oxo-1,3-benzodiazol-1-yl)piperidine-2,6-dione Trifluoroacetate

To a stirred mixture of tert-butyl N-[2-(2-{2-[1-(2,6-dioxopiperidin-3-yl)-3-methyl-2-oxo-1,3-benzodiazol-5-yl]ethoxy}ethoxy)ethyl]carbamate (150 mg, 0.306 mmol) in DCM was added TFA (0.6 mL) dropwise at rt. The resulting mixture was stirred for 1 h at rt. On completion, the reaction mixture was concentrated under reduced pressure. The residue was triturated with diethyl ether (5 mL) to afford the title compound (94 mg, 79% yield) as a white solid. LC/MS (ESI, m/z): [(M+H)]⁺=391.3.

### Example 1.121. Preparation of 6-{[5-(methylcarbamoyl)-4-(phenylamino)pyridin-2-yl]amino}pyridine-3-carboxylic Acid (Intermediate DQ)

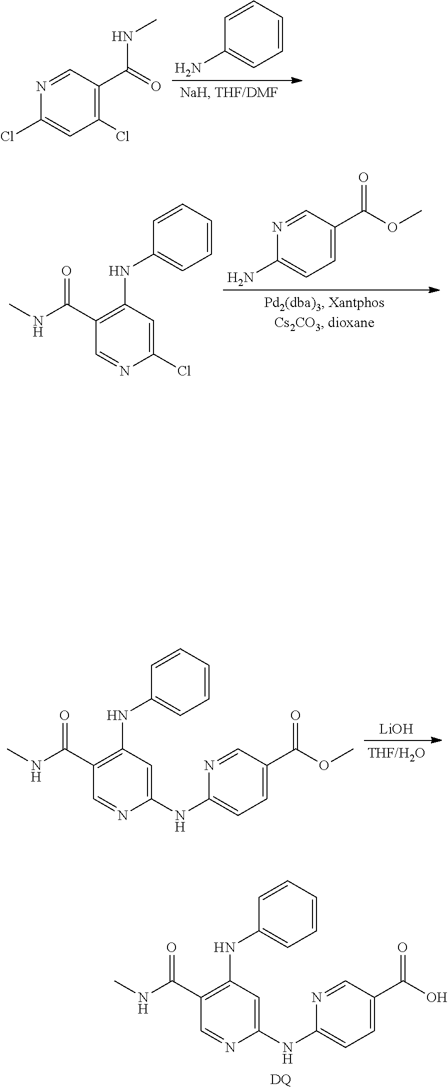

### Step 1—6-Chloro-N-methyl-4-(phenylamino)pyridine-3-carboxamide

To a stirred mixture of aniline (408.780 mg, 4.389 mmol) in THF (5 mL) was added NaH (351.120 mg, 8.778 mmol, 60% dispersion in mineral oil) at 0° C. under nitrogen atmosphere. The resulting mixture was stirred for 30 min. To the above mixture was added 4,6-dichloro-N-methylpyridine-3-carboxamide (600 mg, 3 mmol) at 0° C. The resulting mixture was stirred for additional 3 h at rt. On completion, the reaction was quenched by the addition of sat. NH₄Cl (aq.) (5 mL) at 0° C. The resulting mixture was diluted with water (30 mL), then extracted with EtOAc (3×30 mL). The combined organic layers were washed with brine (3×30 mL), dried over anhydrous Na₂SO₄ and concentrated under reduced pressure. The residue was purified by reverse phase flash chromatography (Column: WelFlash™ C18-I, 20-40 um, 330 g; Eluent A: Water (plus 10 mmol/L FA); Eluent B: ACN; Gradient: 15%-55% B in 40 min; Flow rate: 80 mL/min; Detector: 254 nm; desired fractions were collected at 34% B) and concentrated under reduced pressure to afford the title compound (370 mg, 48% yield) as a white solid. LC/MS (ESI, m/z): [(M+H)]⁺=262.0.

### Step 2—Methyl 6-({4-[(2-methanesulfonylphenyl)amino]-5-(methylcarbamoyl)pyridin-2-yl}amino)pyridine-3-carboxylate

To a stirred mixture of 6-chloro-N-methyl-4-(phenylamino)pyridine-3-carboxamide (370.000 mg, 1.414 mmol) and methyl 6-aminopyridine-3-carboxylate (322.670 mg, 2.121 mmol) in DMA (6 mL) were added BrettPhos Pd G3 (128.160 mg, 0.141 mmol), BrettPhos (75.890 mg, 0.141 mmol) and Cs₂CO₃ (920 mg, 2.428 mmol) in turns at rt under nitrogen atmosphere. The resulting mixture was stirred for 3 h at 120° C. under nitrogen atmosphere. On completion, the reaction mixture was cooled to rt and filtrated. The filtrate was concentrated under reduced pressure. The residue was purified by reverse phase flash chromatography (Column: WelFlash™ C18-I, 20-40 um, 120 g; Eluent A: Water (0.05% TFA); Eluent B: ACN; Gradient: 25%-55% B in 30 min; Flow rate: 70 mL/min; Detector: 254 nm; desired fractions were collected at 39% B) and concentrated under reduced pressure to afford the title compound (340 mg) as a yellow solid. LC/MS (ESI, m/z): [(M+H)]⁺=378.2.

### Step 3—6-{[5-(Methylcarbamoyl)-4-(phenylamino)pyridin-2-yl]amino}pyridine-3-carboxylic Acid

To a stirred mixture of methyl 6-{[5-(methylcarbamoyl)-4-(phenylamino)pyridin-2-yl]amino}pyridine-3-carboxylate (340.00 mg, 0.901 mmol) in THF (8 mL) and H₂O (8 mL) was added LiOH (215.760 mg, 9.009 mmol) at 0° C. under nitrogen atmosphere. The resulting mixture was stirred for 2 h at rt under nitrogen atmosphere. On completion, the mixture was acidified pH to 2 with HCl (aq.). The precipitated solids were collected by filtration. The solid was dried over under reduced pressure to afford the title compound (220 mg) as a white solid. LC/MS (ESI, m/z): [(M+H)]⁺=364.1.

### Example 1.122. Preparation of 6-({4-[(1-methyl-2-oxopyridin-3-yl)amino]-5-(methylcarbamoyl)pyridin-2-yl}amino)pyridine-3-carboxylic Acid (Intermediate DR)

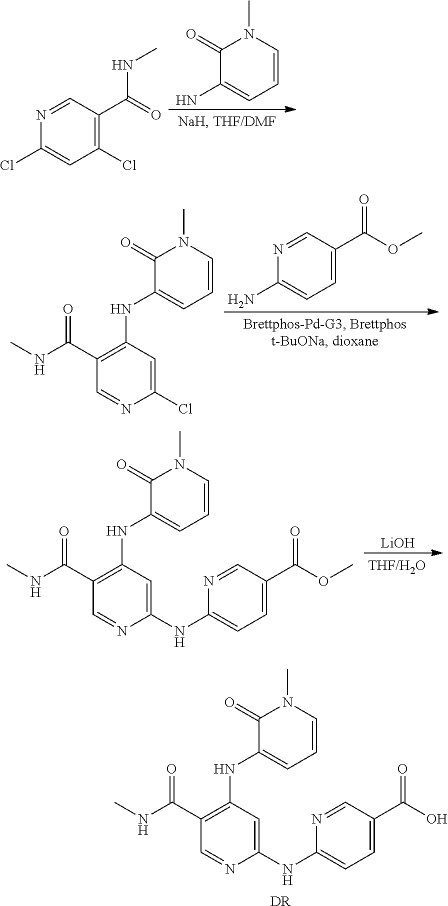

### Step 1—6-Chloro-N-methyl-4-[(1-methyl-2-oxopyridin-3-yl)amino]pyridine-3-carboxamide

To a stirred mixture of 3-amino-1-methylpyridin-2-one (544.910 mg, 4.389 mmol) in THF (5 mL) and DMF (5 mL) were added NaH (588.710 mg, 14.718 mmol, 60% dispersion in mineral oil) at 0° C. under nitrogen atmosphere. The resulting mixture was stirred for 30 min at rt under nitrogen atmosphere. To the above mixture was added 4,6-dichloro-N-methylpyridine-3-carboxamide (600 mg, 3 mmol) 0° C. The resulting mixture was stirred for additional 3 h at 50° C. On completion, the reaction was quenched by the addition of sat. NH₄Cl (aq.) (10 mL) at 0° C. The resulting mixture was cooled to rt and diluted with water (100 mL), then extracted with EtOAc (3×100 mL). The combined organic layers were washed with brine (100 mL), dried over anhydrous Na₂SO₄ and concentrated under reduced pressure. The residue was purified by reverse phase flash chromatography (Column: WelFlash™ C18-I, 20-40 um, 330 g; Eluent A: Water (plus 10 mmol/L FA); Eluent B: ACN; Gradient: 15%-55% B in 40 min; Flow rate: 70 mL/min; Detector: 254 nm; desired fractions were collected at 34% B) and concentrated under reduced pressure to afford the title compound (500.000 mg, 58% yield) as a white solid. LC/MS (ESI, m/z): [(M+H)]⁺=293.1.

### Step 2—Methyl 6-({4-[(1-methyl-2-oxopyridin-3-yl)amino]-5-(methylcarbamoyl)pyridin-2-yl}amino) pyridine-3-carboxylate

To a stirred mixture of 6-chloro-N-methyl-4-[(1-methyl-2-oxopyridin-3-yl)amino]pyridine-3-carboxamide (500 mg, 2 mmol) and methyl 6-aminopyridine-3-carboxylate (389.840 mg, 2.562 mmol) in dioxane (10 mL) were added BrettPhos Pd G3 (154.840 mg, 0.171 mmol), t-BuONa (328.320 mg, 3.416 mmol) and BrettPhos (91.690 mg, 0.171 mmol) in turns at rt under nitrogen atmosphere. The resulting mixture was stirred for 3 h at 100° C. under nitrogen atmosphere. On completion, the reaction mixture was cooled to rt and diluted with water (100 mL), then extracted with EtOAc (100 mL). The combined organic layers were washed with brine (100 mL), dried over anhydrous Na₂SO₄ and concentrated under reduced pressure, then purified by reverse flash chromatography (Column: WelFlash™ C18-I, 20-40 um, 330 g; Eluent A: Water (5 mmol/L NH₄HCO₃); Eluent B: ACN; Gradient: 25%-55% B in 30 min; Flow rate: 80 mL/min; Detector: 254 nm; desired fractions were collected at 35% B) and concentrated under reduced pressure to afford the title compound (350 mg, 54% yield) as a white solid. LC/MS (ESI, m/z): [(M+H)]⁺=409.2.

### Step 3—6-({4-[(1-Methyl-2-oxopyridin-3-yl)amino]-5-(methylcarbamoyl)pyridin-2-yl}amino)pyridine-3-carboxylic Acid

To a stirred mixture of methyl 6-({4-[(1-methyl-2-oxopyridin-3-yl)amino]-5-(methylcarbamoyl)pyridin-2-yl}amino)pyridine-3-carboxylate (50.00 mg, 0.122 mmol) in H₂O (2 mL) and THF (2 mL) was added LiOH (29.28 mg, 1.22 mmol) at rt. The reaction mixture was stirred for 2 h at rt under nitrogen atmosphere. On completion, the reaction mixture was acidified pH to 2 with HCl (aq.). The precipitated solids were collected by filtration and washed with H₂O (2×5 mL). The resulting mixture was concentrated under reduced pressure to afford the title compound (50 mg) as a white solid. LC/MS (ESI, m/z): [(M+H)]⁺=395.2.

### Example 1.123. Preparation of 3-{4-[4-(6-aminopyridin-3-yl)piperazin-1-yl]-3-methyl-2-oxo-1,3-benzodiazol-1-yl}piperidine-2,6-dione (Intermediate DS)

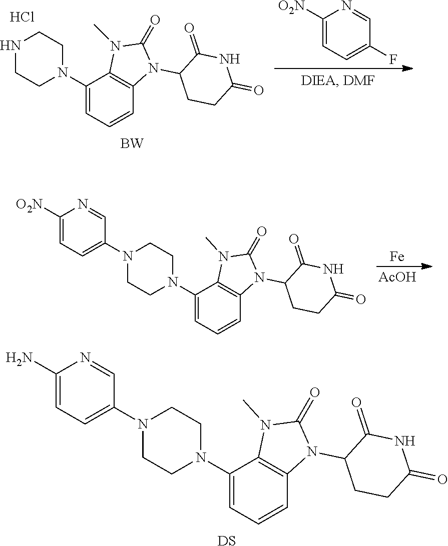

### Step 1—3-{3-Methyl-4-[4-(6-nitropyridin-3-yl)piperazin-1-yl]-2-oxo-1,3-benzodiazol-1-yl}piperidine-2,6-dione

To a stirred solution of 3-[3-methyl-2-oxo-4-(piperazin-1-yl)-1,3-benzodiazol-1-yl]piperidine-2,6-dione hydrochloride (150 mg, 0.4 mmol, Intermediate BW) and 5-fluoro-2-nitropyridine (74.480 mg, 0.524 mmol) in DMF (3 mL) was added DIEA (169.370 mg, 1.311 mmol) at rt. The resulting mixture was stirred for 2 h at 80° C. under nitrogen atmosphere. On completion, the mixture was purified by reverse phase flash chromatography (Column: WelFlash™ C18-I, 20-40 um, 120 g; Eluent A: Water (plus 10 mmol/L FA); Eluent B: ACN; Gradient: 30%-60% B in 25 min; Flow rate: 60 mL/min; Detector: 220/254 nm; desired fractions were collected at 46% B) and concentrated under reduced pressure to afford the title compound (120 mg, 59% yield) as a yellow solid. LC/MS (ESI, m/z): [(M+H)]⁺=466.2.

### Step 2—3-{4-[4-(6-Aminopyridin-3-yl)piperazin-1-yl]-3-methyl-2-oxo-1,3-benzodiazol-1-yl}piperidine-2,6-dione

To a solution of 3-{3-methyl-4-[4-(6-nitropyridin-3-yl)piperazin-1-yl]-2-oxo-1,3-benzodiazol-1-yl}piperidine-2,6-dione (120 mg, 0.26 mmol) in AcOH (3 mL) was added iron (71.99 mg, 1.290 mmol) at 0° C. The resulting mixture was stirred for 16 h at rt. On completion, the reaction mixture was concentrated under reduced pressure. The residue was purified by reverse phase flash chromatography (Column: WelFlash™ C18-I, 20-40 um, 120 g; Eluent A: Water (plus 10 mmol/L FA); Eluent B: ACN; Gradient: 10%-40% B in 25 min; Flow rate: 60 mL/min; Detector: 220/254 nm; desired fractions were collected at 26% B) and concentrated under reduced pressure to afford the title compound (88 mg, 78% yield) as a brown solid. LC/MS (ESI, m/z): [(M+H)]⁺=436.2.

### Example 1.124. Preparation of 6-{[5-(cyclopropylcarbamoyl)-4-{[3-(5-fluoropyrimidin-2-yl)-2-methoxyphenyl]amino}pyridin-2-yl]amino}pyridine-3-carboxylic Acid (Intermediate DT)

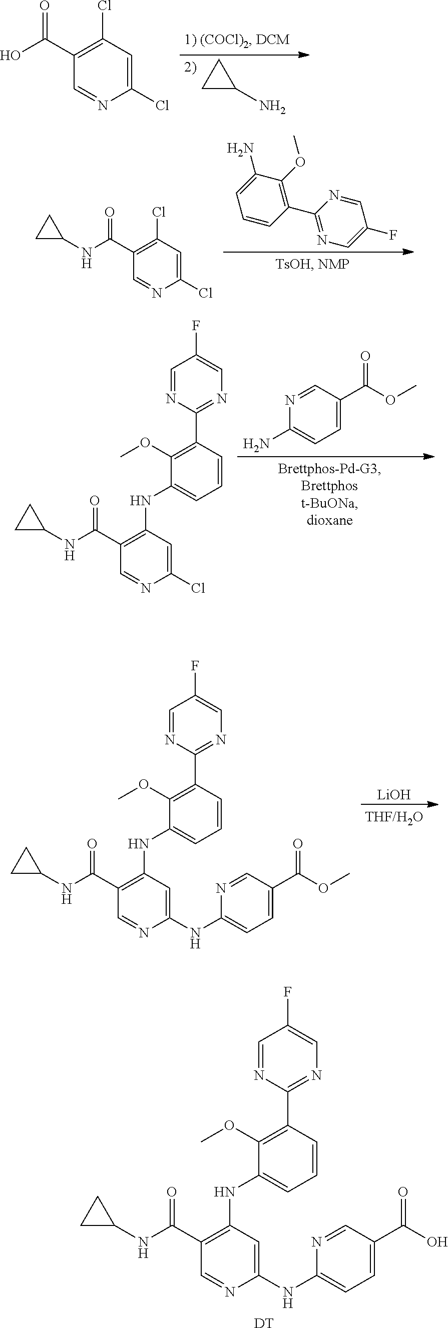

### Step 1—4,6-Dichloro-N-cyclopropylpyridine-3-carboxamide

To a stirred mixture of 4,6-dichloropyridine-3-carboxylic acid (5.00 g, 26.0 mmol) in DCM (30 mL) was added oxalyl chloride (1.59 g, 12.5 mmol) dropwise at 0° C. To the above mixture was added DMF (0.2 mL, 2.6 mmol) dropwise at 0° C. The resulting mixture was stirred for 30 min at rt. To the above mixture was added aminocyclopropane (1.78 g, 31.3 mmol) at 0° C. The resulting mixture was stirred for additional 2 h at rt. On completion, the reaction mixture was diluted with water (100 mL). The resulting mixture was extracted with EtOAc (3×50 mL). The combined organic layers were washed with brine (2×30 mL), and dried over anhydrous Na₂SO₄. After filtration, the filtrate was concentrated under reduced pressure. The residue was purified by silica gel column chromatography, eluted with PE/EA (1:1), to afford the title compound (2.60 g, 43% yield) as a white solid. LC/MS (ESI, m/z): [(M+H)]⁺=231.1, 233.1.

### Step 2—6-Chloro-N-cyclopropyl-4-{[3-(5-fluoropyrimidin-2-yl)-2-methoxyphenyl]amino}pyridine-3-carboxamide

To a stirred mixture of 4,6-dichloro-N-cyclopropylpyridine-3-carboxamide (2.60 g, 11.3 mmol) and 3-(5-fluoropyrimidin-2-yl)-2-methoxyaniline (1.00 g, 4.56 mmol, synthesized via Steps 1-2 of Intermediate K) in NMP (10 mL) was added TsOH (1.18 g, 6.84 mmol) at rt. The resulting mixture was stirred for 4 h at 80° C. On completion, the reaction mixture was cooled to rt and diluted with water (100 mL), then extracted with EtOAc (3×50 mL). The combined organic layers were washed with brine (3×20 mL), and dried over anhydrous Na₂SO₄. After filtration, the filtrate was concentrated under reduced pressure. The residue was purified by silica gel column chromatography, eluted with PE/EA (1:1), to afford the title compound (800 mg, 42% yield) as a white solid. LC/MS (ESI, m/z): [(M+H)]⁺=414.0.

### Step 3—6-{[5-(Cyclopropylcarbamoyl)-4-{[3-(5-fluoropyrimidin-2-yl)-2-methoxyphenyl]amino}pyridin-2-yl]amino}pyridine-3-carboxylate

To a stirred mixture of 6-chloro-N-cyclopropyl-4-{[3-(5-fluoropyrimidin-2-yl)-2-methoxyphenyl]amino}pyridine-3-carboxamide (200 mg, 0.483 mmol) and methyl 6-aminopyridine-3-carboxylate (257.360 mg, 1.690 mmol) in dioxane (8 mL) were added BrettPhos Pd G3 (43.810 mg, 0.048 mmol), BrettPhos (25.940 mg, 0.048 mmol) and t-BuONa (92.890 mg, 0.966 mmol) in turns at rt under nitrogen atmosphere. The resulting mixture was stirred for 16 h at 120° C. under nitrogen atmosphere. On completion, the reaction was purified by reverse phase flash chromatography (Column: WelFlash™ C18-I, 20-40 um, 120 g; Eluent A: Water (5 mmol/L NH₄HCO₃); Eluent B: ACN; Gradient: 25%-55% B in 30 min; Flow rate: 60 mL/min; Detector: 254 nm; desired fractions were collected at 36% B) and concentrated under reduced pressure to afford the title compound (175 mg, 68% yield) as a white solid. LC/MS (ESI, m/z): [(M+H)]⁺=530.2.

### Step 4—6-{[5-(Cyclopropylcarbamoyl)-4-{[3-(5-fluoropyrimidin-2-yl)-2-methoxyphenyl]amino}pyridin-2-yl]amino}pyridine-3-carboxylic Acid

To a stirred mixture of methyl 6-{[5-(cyclopropylcarbamoyl)-4-{[3-(5-fluoropyrimidin-2-yl)-2-methoxyphenyl]amino}pyridin-2-yl]amino}pyridine-3-carboxylate (100 mg, 0.2 mmol) in THF (5 mL) and H₂O (5 mL) was added LiOH (45.230 mg, 1.890 mmol) at 0° C. The resulting mixture was stirred for 2 h at rt. On completion, the mixture was acidified pH to 2 with HCl (aq.). The resulting mixture was concentrated under reduced pressure. The residue was purified by reverse phase flash chromatography (Column: WelFlash™ C18-I, 20-40 um, 120 g; Eluent A: Water (plus 10 mmol/L FA); Eluent B: ACN; Gradient: 20%-50% B in 30 min; Flow rate: 60 mL/min; Detector: 254 nm; desired fractions were collected at 31% B) and concentrated under reduced pressure to afford the title compound (40 mg, 41% yield) as a white solid. LC/MS (ESI, m/z): [(M+H)]⁺=516.2.

### Example 1.125. Preparation of 2-{[5-(Cyclopropylcarbamoyl)-4-{[3-(5-fluoropyrimidin-2-yl)-2-methoxyphenyl]amino}pyridin-2-yl]amino}pyridine-4-carboxylic Acid (Intermediate DU)

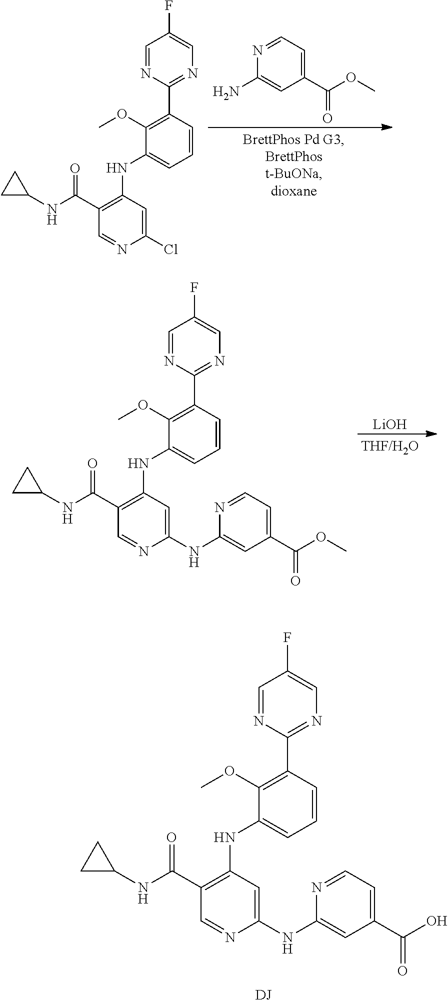 [SMILES: COc1c(Nc2cc(Cl)ncc2C(=O)NC2CC2)cccc1-c1ncc(F)cn1 ; COC(=O)c1ccnc(N)c1 ; COC(=O)c1ccnc(Nc2cc(Nc3cccc(-c4ncc(F)cn4)c3OC)c(C(=O)NC3CC3)cn2)c1 ; "LiOH" ; COc1c(Nc2cc(Nc3cc(C(=O)O)ccn3)ncc2C(=O)NC2CC2)cccc1-c1ncc(F)cn1]

### Step 1—Methyl 2-{[5-(cyclopropylcarbamoyl)-4-{[3-(5-fluoropyrimidin-2-yl)-2-methoxyphenyl] amino} pyridin-2-yl]amino}pyridine-4-carboxylate

To a stirred mixture of 6-chloro-N-cyclopropyl-4-{[3-(5-fluoropyrimidin-2-yl)-2-methoxyphenyl]amino}pyridine-3-carboxamide (200 mg, 0.5 mmol, synthesized via Steps 1-2 of Intermediate DT) and methyl 2-aminopyridine-4-carboxylate (257.360 mg, 1.690 mmol) in dioxane (8 mL) were added BrettPhos Pd G3 (43.810 mg, 0.048 mmol), BrettPhos (25.940 mg, 0.048 mmol) and t-BuONa (92.890 mg, 0.966 mmol) in turns at rt under nitrogen atmosphere. The resulting mixture was then stirred for 16 h at 130° C. under nitrogen atmosphere. On completion, the reaction mixture was cooled to rt and concentrated under reduced pressure. The residue was purified by reverse phase flash chromatography (Column: WelFlash™ C18-I, 20-40 um, 120 g; Eluent A: Water (plus 10 mmol/L FA); Eluent B: ACN; Gradient: 20%-55% B in 35 min; Flow rate: 70 mL/min; Detector: 254 nm; desired fractions were collected at 40% B) and concentrated under reduced pressure to afford the title compound (150 mg, 59% yield) as a white solid. LC/MS (ESI, m/z): [(M+H)]⁺=530.2.

### Step 2—2-{[5-(cyclopropylcarbamoyl)-4-{[3-(5-fluoropyrimidin-2-yl)-2-methoxyphenyl]amino}pyridin-2-yl]amino}pyridine-4-carboxylic Acid

To a stirred mixture of methyl 2-{[5-(cyclopropylcarbamoyl)-4-{[3-(5-fluoropyrimidin-2-yl)-2-methoxyphenyl]amino}pyridin-2-yl]amino}pyridine-4-carboxylate (70 mg, 0.1 mmol) in THF (4 mL) and H₂O (4 mL) was added LiOH (31.66 mg, 1.320 mmol) at 0° C. The resulting mixture was stirred for 2 h at rt. On completion, the reaction mixture was acidified pH to 3 with HCl (aq.). The resulting precipitate was filtered, and the filter cake was washed with water (2×5 mL), then dried under vacuum to afford the title compound (25 mg, crude) as a white solid. LC/MS (ESI, m/z): [(M+H)]⁺=516.2.

### Example 1.126. Preparation of 1-(2,6-dioxopiperidin-3-yl)-3-methyl-2-oxo-1,3-benzodiazole-5-carbaldehyde (Intermediate DV)

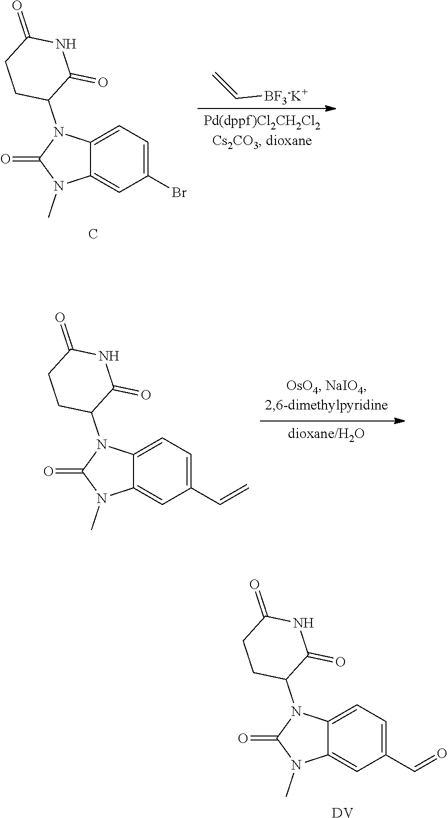

### Step 1—3-(3-Methyl-2-oxo-5-vinyl-2,3-dihydro-1H-benzo[d]imidazol-1-yl)piperidine-2,6-dione

To a solution of 3-(5-bromo-3-methyl-2-oxo-benzimidazol-1-yl)piperidine-2,6-dione (5 g, 15 mmol, Intermediate C) and potassium; trifluoro(vinyl)boranuide (5.94 g, 44.3 mmol) in dioxane (150 mL) was added cesium carbonate (2 M, 14.8 mL) and [1,1-bis(diphenylphino)ferrocene]pallidum,(II)chloride (1.21 g, 1.48 mmol). The mixture was stirred at 85° C. for 12 hrs. On completion, the reaction mixture was diluted with water (50 mL) and extracted with ethyl acetate (50 mL×2). The combined organic layers were washed with brine (50 mL×2), dried over with anhydrous sodium sulfate, filtered and concentrated under reduced pressure to give a residue. The residue was purified by column chromatography (SiO₂, Petroleum ether/Ethyl acetate/THF=4/4/1 to 1/1/1) to give the title compound (3.8 g, 84% yield) as a yellow solid. ¹H NMR (400 MHz, DMSO-d6) δ=11.11 (s, 1H), 7.37 (s, 1H), 7.18-7.02 (m, 2H), 6.75 (dd, J=10.8, 17.6 Hz, 1H), 5.81 (d, J=17.6 Hz, 1H), 5.37 (dd, J=5.2, 12.8 Hz, 1H), 5.18 (d, J=11.2 Hz, 1H), 3.37 (s, 3H), 2.97-2.84 (m, 1H), 2.80-2.59 (m, 2H), 2.10-2.00 (m, 1H).

### Step 2—1-(2,6-Dioxopiperidin-3-yl)-3-methyl-2-oxo-2,3-dihydro-1H-benzo[d]imidazole-5-carbaldehyde

To a solution of 3-(3-methyl-2-oxo-5-vinyl-benzimidazol-1-yl)piperidine-2,6-dione (2.8 g, 9.8 mmol) in dioxane (30 mL) and water (30 mL) was added sodium periodate (8.40 g, 39.2 mmol), 2,6-dimethylpyridine (2.10 g, 19.6 mmol) and osmium tetroxide (499 mg, 1.96 mmol) at 0° C. The mixture was stirred at 0° C. for 1 hour. On completion, the reaction mixture was quenched by addition saturated sodium thiosulfate aqueous solution (30 mL), and then diluted with water (60 mL), and extracted with ethyl acetate (250 mL×2). The combined organic layers were washed with brine (150 mL×2), dried over anhydrous sodium sulfate, filtered and concentrated under reduced pressure to give a residue. The residue was washed with ethyl acetate (30 mL) to give the title compound (2.2 g, 78% yield) as an off-white solid. ¹H NMR (400 MHz, DMSO-d6) δ=11.18 (s, 1H), 9.95 (s, 1H), 7.86-7.60 (m, 2H), 7.37 (d, J=8.4 Hz, 1H), 5.49 (dd, J=5.6, 12.8 Hz, 1H), 3.43 (s, 3H), 2.98-2.85 (m, 1H), 2.81-2.72 (m, 1H), 2.70-2.60 (m, 1H), 2.08 (dd, J=4.8, 10.4 Hz, 1H).

### Example 1.127. Preparation of 4-{[3-(5-fluoropyrimidin-2-yl)-2-methoxyphenyl]amino}-N-methyl-6-{[5-(piperidin-4-yl)pyridin-2-yl]amino}pyridine-3-carboxamide (Intermediate DW)

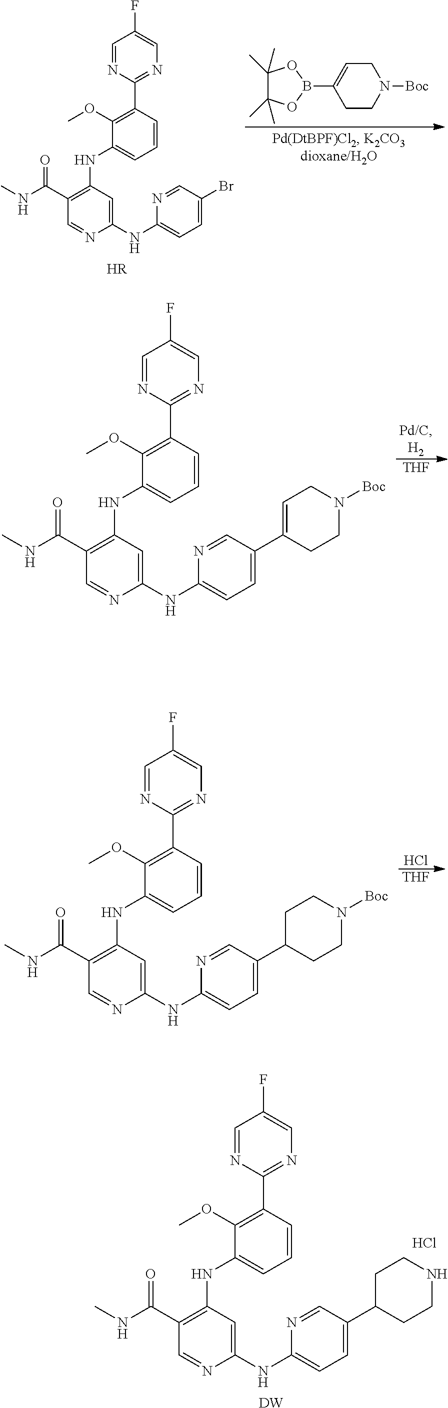

### Step 1—Tert-butyl 6-[(4-{[3-(5-fluoropyrimidin-2-yl)-2-methoxyphenyl]amino}-5-(methylcarbamoyl) pyridine-2-yl)amino]-3′,6′-dihydro-2′H-[3,4′-bipyridine]-1′-carboxylate

To a stirred mixture of 6-[(5-bromopyridin-2-yl)amino]-4-{[3-(5-fluoropyrimidin-2-yl)-2-methoxyphenyl]amino}-N-methylpyridine-3-carboxamide (500.0 mg, 0.954 mmol, Intermediate HR) and tert-butyl 4-(4,4,5,5-tetramethyl-1,3,2-dioxaborolan-2-yl)-3,6-dihydro-2H-pyridine-1-carboxylate (442.270 mg, 1.431 mmol, CAS #286961-14-6) in dioxane (10 mL) and H₂O (2 mL) were added Pd(dtbpf)Cl₂ (61.37 mg, 0.095 mmol) and K₂CO₃ (263.570 mg, 1.908 mmol) in turns at rt under nitrogen atmosphere. The resulting mixture was stirred for 3 h at 90° C. under nitrogen atmosphere. On completion, the reaction mixture was concentrated under reduced pressure. The residue was purified by reverse phase flash chromatography (Column: WelFlash™ C18-I, 20-40 um, 330 g; Eluent A: Water (10 mmol/L NH₄HCO₃); Eluent B: ACN; Gradient: 25%-55% B in 30 min; Flow rate: 80 mL/min; Detector: 254 nm; desired fractions were collected at 36% B) and concentrated under reduced pressure to afford the title compound (570 mg, 95% yield) as a white solid. LC/MS (ESI, m/z): [(M+H)]⁺=627.3.

### Step 2—Tert-butyl 4-{6-[(4-{[3-(5-fluoropyrimidin-2-yl)-2-methoxyphenyl]amino}-5-(methylcarbamoyl) pyridin-2-yl)amino]pyridin-3-yl}piperidine-1-carboxylate

To a solution of tert-butyl 6-[(4-{[3-(5-fluoropyrimidin-2-yl)-2-methoxyphenyl]amino}-5-(methylcarbamoyl)pyridin-2-yl)amino]-3′,6′-dihydro-2′H-[3,4′-bipyridine]-1′-carboxylate (570 mg, 0.910 mmol) in THF (10 mL) was added 10 wt % Pd/C (57.0 mg) under nitrogen atmosphere. The reaction system was degassed under vacuum and purged with H₂ several times, then it was hydrogenated under H₂ balloon (1 atm) at rt for 16 h. After completion of the reaction, Pd/C was filtered off through celite and the corresponding filtrate was concentrated under reduced pressure. The residue was purified by reverse phase flash chromatography (Column: WelFlash™ C18-I, 20-40 um, 330 g; Eluent A: Water (10 mmol/L NH₄HCO₃); Eluent B: ACN; Gradient: 25%-55% B in 30 min; Flow rate: 80 mL/min; Detector: 254 nm; desired fractions were collected at 40% B) and concentrated under reduced pressure to afford the title compound (400 mg, 70% yield) as a light yellow solid. LC/MS (ESI, m/z): [(M+H)]⁺=629.2.

### Step 3—4-{[3-(5-Fluoropyrimidin-2-yl)-2-methoxyphenyl]amino}-N-methyl-6-{[5-(piperidin-4-yl)pyridin-2-yl]amino}pyridine-3-carboxamide Hydrochloride

To a stirred mixture of tert-butyl 4-{6-[(4-{[3-(5-fluoropyrimidin-2-yl)-2-methoxyphenyl]amino}-5-(methylcarbamoyl)pyridin-2-yl)amino]pyridin-3-yl} piperidine-1-carboxylate (400 mg, 0.6 mmol) in DCM (7.5 mL) was added 4 M HCl (gas) in 1,4-dioxane (1.5 mL) at rt. The resulting mixture was stirred for 2 h at rt under nitrogen atmosphere. On completion, the reaction mixture was concentrated under reduced pressure. The residue was triturated with Et₂O to afford the title compound (230.0 mg) as a light yellow solid. LC/MS (ESI, m/z): [(M+H)]⁺=529.2.

### Example 1.128. Preparation of 3-{3-methyl-4-[3-(methylamino)azetidin-1-yl]-2-oxo-1,3-benzodiazol-1-yl}piperidine-2,6-dione (Intermediate DX)

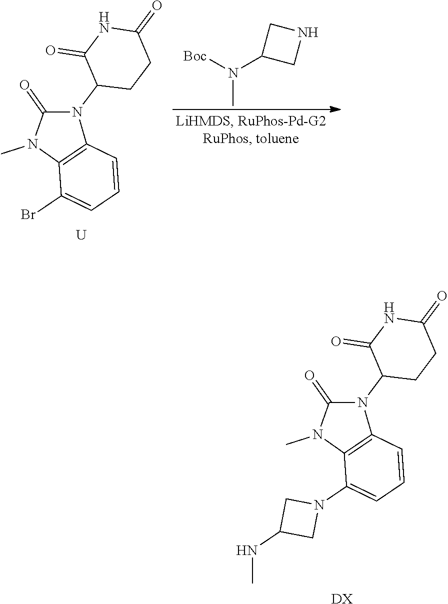

To a stirred mixture of 3-(4-bromo-3-methyl-2-oxo-1,3-benzodiazol-1-yl)piperidine-2,6-dione (600 mg, 2 mmol, Intermediate U) and tert-butyl N-(azetidin-3-yl)-N-methylcarbamate (495.71 mg, 2.661 mmol) in methylbenzene (15 mL) were added RuPhos-PdCl-2nd G (275.99 mg, 0.355 mmol), RuPhos (165.59 mg, 0.355 mmol) and LiHMDS (10.65 mL, 10.644 mmol) in turns at rt under nitrogen atmosphere. The resulting mixture was stirred for 2 h at 80° C. under nitrogen atmosphere. On completion, the mixture was cooled to rt and was neutralized pH to 4 with FA. The resulting mixture was filtered, and the filter cake was washed with DCM (3×7 mL). The filtrate was concentrated under reduced pressure. The residue was purified by reverse phase flash (Column: WelFlash™ C18-I, 20-40 um, 120 g; Eluent A: Water (10 mmol/L FA); Eluent B: ACN; Gradient: 25%-55% B in 25 min; Flow rate: 60 mL/min; Detector: 254 nm; desired fractions were collected at 35% B) and concentrated under reduced pressure to afford the title compound (128 mg, 21% yield) as a white solid. LC/MS (ESI, m/z): [(M+H)]⁺=344.2.

### Example 1.129. Preparation of 3-{3-methyl-5-[3-(methylamino)pyrrolidin-1-yl]-2-oxo-1,3-benzodiazol-1-yl}piperidine-2,6-dione (Intermediate DY)

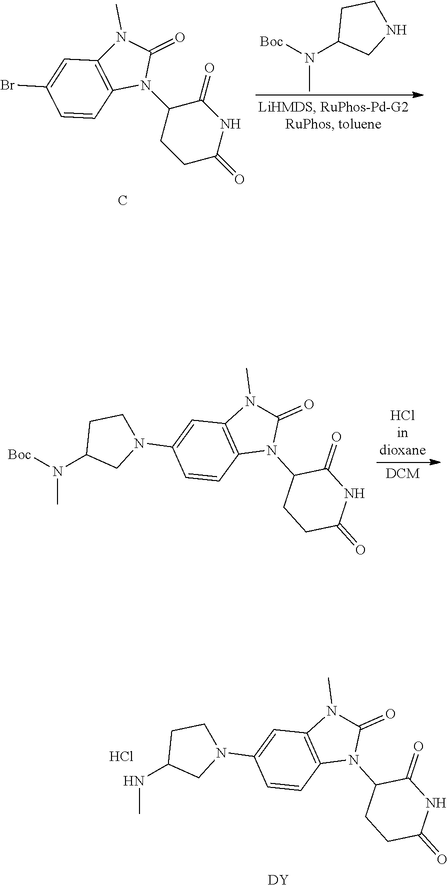

### Step 1—Tert-butyl N-{1-[1-(2,6-dioxopiperidin-3-yl)-3-methyl-2-oxo-1,3-benzodiazol-5-yl]pyrrolidin-3-yl}-N-methylcarbamate

To a stirred solution of 3-(5-bromo-3-methyl-2-oxo-1,3-benzodiazol-1-yl)piperidine-2,6-dione (500 mg, 2 mmol, Intermediate C) and tert-butyl N-methyl-N-(pyrrolidin-3-yl)carbamate (592.270 mg, 2.958 mmol) in toluene (10 mL) were added RuPhos-PdCl-2nd G (229.990 mg, 0.296 mmol), RuPhos (138 mg, 0.296 mmol) and LiHMDS (8.874 mL, 8.874 mmol) at rt under nitrogen atmosphere. The resulting mixture was stirred under nitrogen atmosphere at 80° C. for 2 h. On completion, the reaction mixture was cooled to rt and was acidified to pH 3 with FA. The resulting mixture was concentrated under reduced pressure. The residue was purified by reverse phase flash chromatography (Column: WelFlash™ C18-I, 20-40 um, 120 g; Eluent A: Water (plus 10 mmol/L FA); Eluent B: ACN; Gradient: 22%-55% B in 25 min; Flow rate: 60 mL/min; Detector: 220/254 nm; desired fractions were collected at 50% B) and concentrated under reduced pressure to afford the title compound (500 mg, 74% yield) as a yellow solid. LC/MS (ESI, m/z): [(M+H)]⁺=458.2.

### Step 2—3-{3-Methyl-5-[3-(methylamino)pyrrolidin-1-yl]-2-oxo-1,3-benzodiazol-1-yl}piperidine-2,6-dione Hydrochloride

To a stirred solution of tert-butyl N-{1-[1-(2,6-dioxopiperidin-3-yl)-3-methyl-2-oxo-1,3-benzodiazol-5-yl]pyrrolidin-3-yl}-N-methylcarbamate (500 mg, 1 mmol) in DCM (5 mL) was added a solution of 4 M HCl (gas) in 1,4-dioxane (5 mL) at rt. The resulting mixture was stirred for 1 h at rt. On completion, the reaction mixture was concentrated under reduced pressure. The residue was triturated with Et₂O (10 mL) to afford the title compound (400 mg) as a pink solid. LC/MS (ESI, m/z): [(M+H)]⁺=358.2.

### Example 1.130. Preparation of 3-{3-methyl-5-[3-(methylamino)azetidin-1-yl]-2-oxo-1,3-benzodiazol-1-yl}piperidine-2,6-dione (Intermediate DZ)

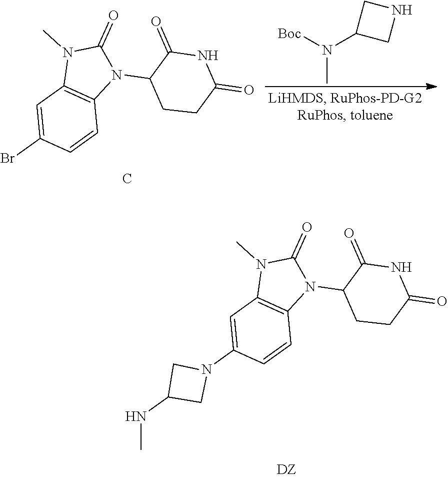

To a stirred mixture of 3-(5-bromo-3-methyl-2-oxo-1,3-benzodiazol-1-yl)piperidine-2,6-dione (600 mg, 2 mmol, Intermediate C) and tert-butyl N-(azetidin-3-yl)-N-methylcarbamate (660.95 mg, 3.548 mmol) in methylbenzene (10 mL) were added RuPhos-PdCl-2nd G (275.99 mg, 0.355 mmol), RuPhos (165.59 mg, 0.355 mmol) and LiHMDS in THF (10.65 mL, 10.644 mmol) in turns at rt under nitrogen atmosphere. The resulting mixture was stirred for 2 h at 80° C. under nitrogen atmosphere. On completion, the mixture was acidified pH to 4 with FA (aq.). The resulting mixture was filtered, and the filter cake was washed with DCM (3×10 mL). The filtrate was concentrated under reduced pressure. The residue was purified by reverse phase flash (Column: WelFlash™ C18-I, 20-40 um, 330 g; Eluent A: Water (10 mmol/L FA); Eluent B: ACN; Gradient: 20%-60% B in 35 min; Flow rate: 80 mL/min; Detector: 254 nm; desired fractions were collected at 35% B) and concentrated under reduced pressure to afford the title compound (200 mg, 33% yield) as a brown solid. LC/MS (ESI, m/z): [(M+H)]⁺=344.1.

### Example 1.131. Preparation of 3-{3-methyl-4-[methyl(piperidin-4-yl)amino]-2-oxo-1,3-benzodiazol-1-yl}piperidine-2,6-dione (Intermediate EA)

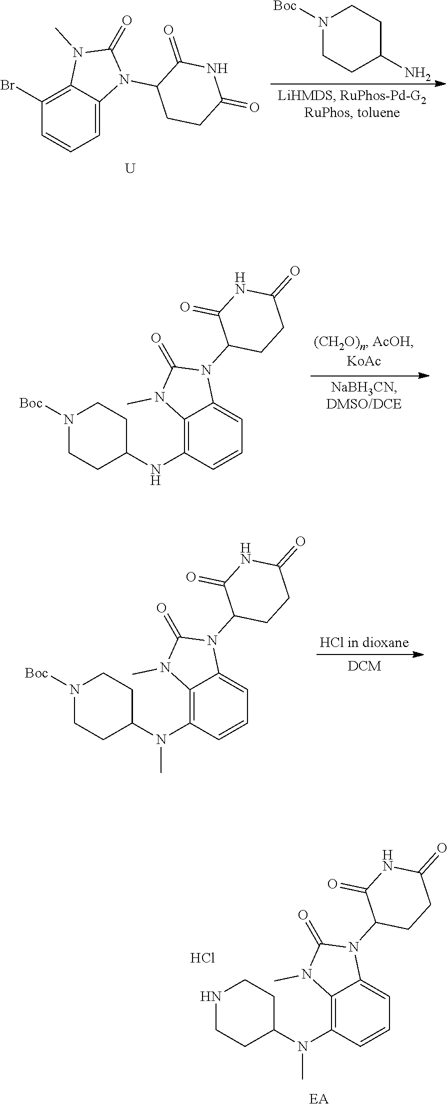

### Step 1—Tert-butyl 4-{[1-(2,6-dioxopiperidin-3-yl)-3-methyl-2-oxo-1,3-benzodiazol-4-yl]amino}piperidine-1-carboxylate

To a stirred mixture of 3-(4-bromo-3-methyl-2-oxo-1,3-benzodiazol-1-yl)piperidine-2,6-dione (1 g, 3 mmol, Intermediate U) and tert-butyl 4-aminopiperidine-1-carboxylate (1.18 g, 5.914 mmol) in methylbenzene (15 mL) were added RuPhos-PdCl-2nd G (0.46 g, 0.59 mmol), RuPhos (0.28 g, 0.59 mmol) and LiHMDS (17.74 mL, 17.74 mmol) in turns at rt under nitrogen atmosphere. The resulting mixture was stirred for 2 h at 80° C. under nitrogen atmosphere. On completion, the reaction was acidified to pH 4 with FA. The resulting mixture was filtered, then the filter cake was washed with DCM (3×20 mL). The filtrate was concentrated under reduced pressure. The residue was purified by reverse phase flash (Column: WelFlash™ C18-I, 20-40 um, 330 g; Eluent A: Water (10 mmol/L FA); Eluent B: ACN; Gradient: 25%-70% B in 30 min; Flow rate: 80 mL/min; Detector: 254 nm; desired fractions were collected at 40% B) and concentrated under reduced pressure to afford the title compound (800 mg, 59% yield) as a brown solid. LC/MS (ESI, m/z): [(M+H)]⁺=458.2.

### Step 2—Tert-butyl 4-{[1-(2,6-dioxopiperidin-3-yl)-3-methyl-2-oxo-1,3-benzodiazol-4-yl](methyl)amino}piperidine-1-carboxylate

To a stirred mixture of tert-butyl 4-{[1-(2,6-dioxopiperidin-3-yl)-3-methyl-2-oxo-1,3-benzodiazol-4-yl]amino}piperidine-1-carboxylate (550 mg, 1.20 mmol) and methoxymethanol amine (264.78 mg, 6.010 mmol) in DMSO (3 mL) and DCE (3 mL) were added KOAc (353.93 mg, 3.606 mmol) and AcOH (0.69 mL, 12 mmol) in turns at rt under nitrogen atmosphere. The resulting mixture was stirred for 30 min at rt under nitrogen atmosphere. To the above mixture was added NaBH₃CN (377.70 mg, 6.010 mmol) at 0° C. The resulting mixture was stirred for additional 4 h at 60° C. On completion, the reaction mixture was concentrated under reduced pressure. The residue was purified by reverse phase flash (Column: WelFlash™ C18-I, 20-40 um, 330 g; Eluent A: Water (10 mmol/L FA); Eluent B: ACN; Gradient: 25%-55% B in 25 min; Flow rate: 80 mL/min; Detector: 254 nm; desired fractions were collected at 45% B) and concentrated under reduced pressure to afford the title compound (300 mg, 53% yield) as a white solid. LC/MS (ESI, m/z): [(M+H)]⁺=472.2.

### Step 3—3-{3-Methyl-4-[methyl(piperidin-4-yl)amino]-2-oxo-1,3-benzodiazol-1-yl}piperidine-2,6-dione Hydrochloride

To a stirred mixture of tert-butyl 4-{[1-(2,6-dioxopiperidin-3-yl)-3-methyl-2-oxo-1,3-benzodiazol-4-yl](methyl)amino}piperidine-1-carboxylate (300 mg, 0.6 mmol) in DCM (2 mL) was added 4 M HCl (gas) in 1,4-dioxane (1 mL) dropwise at rt under nitrogen atmosphere. The resulting mixture was stirred for 1 h at rt under nitrogen atmosphere. On completion, the reaction mixture was concentrated under reduced pressure. The residue was trituration with Et₂O to afford the title compound (250 mg) as a white solid. LC/MS (ESI, m/z): [(M+H)]⁺=372.2.

### Example 1.132. Preparation of 3-{3-methyl-4-[3-(methylamino)piperidin-1-yl]-2-oxo-1,3-benzodiazol-1-yl}piperidine-2,6-dione (Intermediate EB)

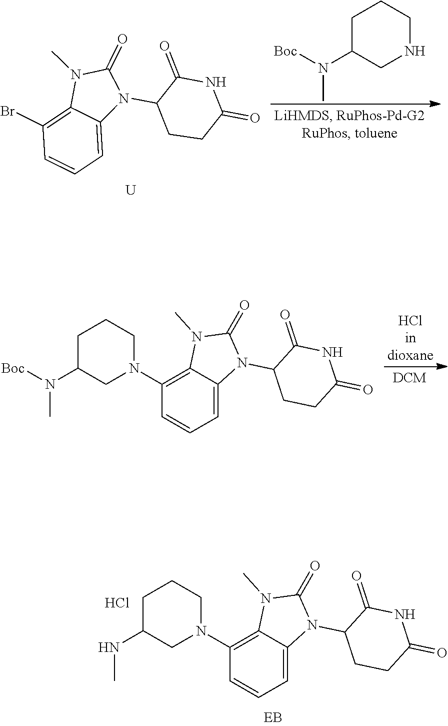

### Step 1—Tert-butyl N-{1-[1-(2,6-dioxopiperidin-3-yl)-3-methyl-2-oxo-1,3-benzodiazol-4-yl]piperidin-3-yl}-N-methylcarbamate

To a stirred solution of 3-(4-bromo-3-methyl-2-oxo-1,3-benzodiazol-1-yl)piperidine-2,6-dione (500 mg, 2 mmol, Intermediate U) and tert-butyl N-methyl-N-(piperidin-3-yl)carbamate (633.75 mg, 2.958 mmol) in toluene (10 mL) were added RuPhos-PdCl-2nd G (229.99 mg, 0.296 mmol), LiHMDS (1484.47 mg, 8.874 mmol) and RuPhos (138.00 mg, 0.296 mmol) in turns at rt. The resulting mixture was stirred for 3 h at 80° C. under nitrogen atmosphere. On completion, the reaction mixture was cooled to rt and was acidified to pH 3 with FA. The resulting mixture was filtered, the filter cake was washed with acetonitrile (3×10 mL). The filtrate was concentrated under reduced pressure. The residue was purified by reverse phase flash chromatography (Column: WelFlash™ C18-I, 20-40 um, 330 g; Eluent A: Water (plus 10 mmol/L FA); Eluent B: ACN; Gradient: 30%-70% B in 25 min; Flow rate: 80 mL/min; Detector: 220/254 nm; desired fractions were collected at 50% B) and concentrated under reduced pressure to afford the title compound (600 mg, 86% yield) as a white solid. LC/MS (ESI, m/z): [(M+H)]⁺=472.2.

### Step 2—3-{3-Methyl-4-[3-(methylamino)piperidin-1-yl]-2-oxo-1,3-benzodiazol-1-yl}piperidine-2,6-dione Hydrochloride

To a stirred solution of tert-butyl N-{1-[1-(2,6-dioxopiperidin-3-yl)-3-methyl-2-oxo-1,3-benzodiazol-4-yl]piperidin-3-yl}-N-methylcarbamate (500 mg, 1 mmol) in DCM (10 mL) was added 4 M HCl (gas) in 1,4-dioxane (4 mL) at rt. The resulting mixture was stirred for 2 h at rt. On completion, the reaction mixture was concentrated under reduced pressure. The residue was purified by trituration with Et₂O to afford the title compound (380 mg) as a white solid. LC/MS (ESI, m/z): [(M+H)]⁺=372.1.

### Example 1.133. Preparation of 3-(3-methyl-5-(3-(methylamino)piperidin-1-yl)-2-oxo-2,3-dihydro-1H-benzo[d]imidazol-1-yl)piperidine-2,6-dione (Intermediate EC)

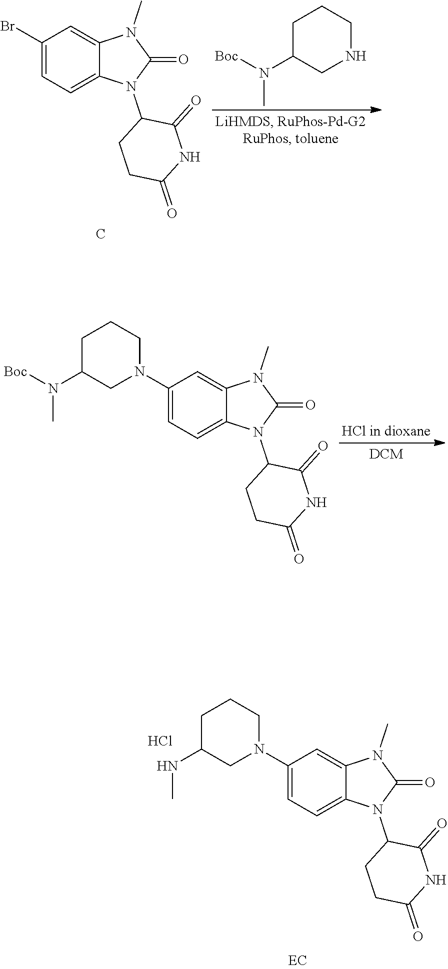

### Step 1: 6-[(5-{[4-({3-[1-(2,6-Dioxopiperidin-3-yl)-3-methyl-2-oxo-1,3-benzodiazol-4-yl]prop-2-yn-1-yl}oxy)piperidin-1-yl]methyl}pyridin-2-yl)amino]-4-{[3-(5-fluoropyrimidin-2-yl)-2-methoxyphenyl]amino}-N-methylpyridine-3-carboxamide

To a stirred solution of 3-(5-bromo-3-methyl-2-oxo-1,3-benzodiazol-1-yl)piperidine-2,6-dione (500 mg, 2 mmol, Intermediate C) and tert-butyl N-methyl-N-(piperidin-3-yl)carbamate (633.75 mg, 2.957 mmol) in toluene (10 mL) were added RuPhos-PdCl-2nd G (229.99 mg, 0.296 mmol), LiHMDS (1484.47 mg, 8.874 mmol) and RuPhos (138.00 mg, 0.296 mmol) in turns at rt. The resulting mixture was stirred for 2 h at 80° C. under nitrogen atmosphere. On completion, the reaction was cooled to rt and was acidified to pH 3 with FA. The resulting mixture was filtered, the filter cake was washed with acetonitrile (3×10 mL). The filtrate was concentrated under reduced pressure. The residue was purified by reverse phase flash chromatography (Column: WelFlash™ C18-I, 20-40 um, 330 g; Eluent A: Water (plus 10 mmol/L FA); Eluent B: ACN; Gradient: 30%-70% B in 25 min; Flow rate: 80 mL/min; Detector: 220/254 nm; desired fractions were collected at 61% B) and concentrated under reduced pressure to afford the title compound (330 mg, 47% yield) as a white solid. LC/MS (ESI, m/z): [(M+H)]⁺=472.1.

### Step 2—3-(3-Methyl-5-(3-(methylamino)piperidin-1-yl)-2-oxo-2,3-dihydro-1H-benzo[d]imidazol-1-yl)piperidine-2,6-dione Hydrochloride

To a stirred solution of tert-butyl N-{1-[1-(2,6-dioxopiperidin-3-yl)-3-methyl-2-oxo-1,3-benzodiazol-5-yl]piperidin-3-yl}-N-methylcarbamate (330 mg, 0.699 mmol) and in DCM (6 mL) was added 4 M HCl (gas) in 1,4-dioxane (2 mL) at rt. The resulting mixture was stirred for 2 h at rt. On completion, the reaction mixture was concentrated under reduced pressure. The residue was purified by trituration with Et₂O (10 mL) to afford the title compound (220 mg) as a white solid. LC/MS (ESI, m/z): [(M+H)]⁺=372.3.

### Example 1.134. Preparation of 3-{3-methyl-2-oxo-4-[3-(piperidin-4-yloxy)prop-1-yn-1-yl]-1,3-benzodiazol-1-yl}piperidine-2,6-dione (Intermediate ED) (TK2-409)

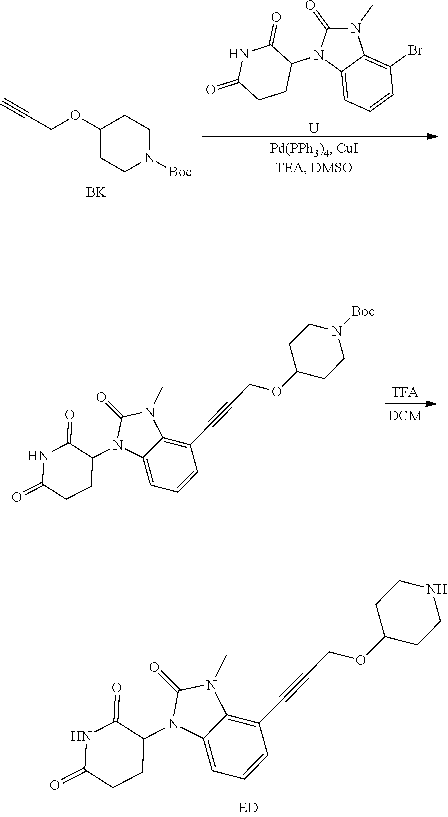

### Step 1—Tert-butyl 4-((3-(1-(2,6-dioxopiperidin-3-yl)-3-methyl-2-oxo-2,3-dihydro-1H-benzo[d]imidazol-4-yl)prop-2-yn-1-yl)oxy)piperidine-1-carboxylate

A mixture of tert-butyl 4-prop-2-ynoxypiperidine-1-carboxylate (7.43 g, 31.1 mmol, Intermediate BK), 3-(4-bromo-3-methyl-2-oxo-benzimidazol-1-yl)piperidine-2,6-dione (7.00 g, 20 mmol, Intermediate U), CuI (394 mg, 2.07 mmol), Pd(PPh₃)₄ (2.39 g, 2.07 mmol) and TEA (20.9 g, 207 mmol, 28.8 mL) in DMSO (120 mL) was degassed and purged with N₂ three times in glove box. The mixture was stirred at 80° C. for 12 hr under N₂ atmosphere. On completion, H₂O (300 mL) was added into the mixture, and the solution was extracted with EA (200 mL×3). The combined organic layer was washed with brine (300 mL) and dried over Na₂SO₄, then the solvent was removed under reduced pressure to give a residue. The residue was purified by column chromatography (SiO₂, PE:EA=3:1 to 0:1, Rf=0.6) to give the crude product (10 g, 81% purity). The product was repurified by reversed-phase HPLC (0.1% FA condition) to give the title compound (6.38 g, 60% yield) as off-white solid. ¹H NMR (400 MHz, CDCl₃-d) δ=8.23 (s, 1H), 7.18-7.16 (d, J=7.6 Hz, 1H), 7.02-6.98 (t, J=8 Hz, 1H), 6.78-6.76 (d, J=8 Hz, 1H), 5.23-5.19 (dd, J=5.2, 12.8 Hz, 1H), 4.47 (s, 2H), 3.78-3.76 (m, 6H), 3.16-3.10 (m, 2H), 2.93-2.75 (m, 3H), 2.26-2.24 (m, 1H), 1.90-1.88 (m, 2H), 1.64 (m, 2H), 1.47 (s, 9H).

### Step 2—3-{3-Methyl-2-oxo-4-[3-(piperidin-4-yloxy)prop-1-yn-1-yl]-1,3-benzodiazol-1-yl}piperidine-2,6-dione Trifluoroacetate

To a stirred solution of tert-butyl 4-({3-[1-(2,6-dioxopiperidin-3-yl)-3-methyl-2-oxo-1,3-benzodiazol-4-yl]prop-2-yn-1-yl}oxy)piperidine-1-carboxylate (75 mg, 0.15 mmol) and in DCM (3 mL) was added TFA (1 mL) at rt. The resulting mixture was stirred for 1 h at rt. On completion, the resulting mixture was concentrated under reduced pressure. The residue was purified by trituration with Et₂O (10 mL) to afford the title compound (50 mg) as a white solid. LC/MS (ESI, m/z): [(M+H)]⁺=397.4.

### Example 1.135. Preparation of 3-[3-methyl-2-oxo-5-(piperazin-1-ylmethyl)-1,3-benzodiazol-1-yl]piperidine-2,6-dione (Intermediate EE)

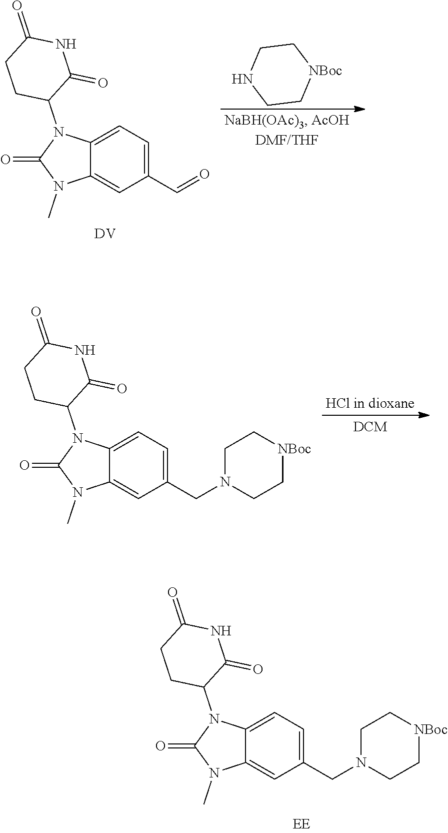

### Step 1—Tert-butyl 4-((1-(2,6-dioxopiperidin-3-yl)-3-methyl-2-oxo-2,3-dihydro-1H-benzo[d]imidazol-5-yl)methyl)piperazine-1-carboxylate

To a solution of 1-(2,6-dioxo-3-piperidyl)-3-methyl-2-oxo-benzimidazole-5-carbaldehyde (2 g, 7 mmol, Intermediate DV) and tert-butyl piperazine-1-carboxylate (1.95 g, 10.4 mmol) in tetrahydrofuran (10 mL) and dimethylformamide (10 mL) was added acetic acid (2.09 g, 34.8 mmol) at 25° C. for 0.5 hour. Then sodium triacethoxy borohydride (4.43 g, 20.8 mmol) was added at 25° C. and the mixture was stirred at 25° C. for 2.5 hours. On completion, the reaction mixture was partitioned between water (50 mL) and ethyl acetate (200 mL). The organic phase was separated, washed with brine (100 mL), dried over with Na₂SO₄, filtered and concentrated under reduced pressure to give a residue. The crude product was purified by reversed-phase HPLC (0.1% FA condition) to give the title compound (1.67 g, 52% yield) as an off-white solid. ¹H NMR (400 MHz, DMSO-d6) δ=11.11 (s, 1H), 7.24 (s, 1H), 7.16-7.00 (m, 2H), 5.38 (dd, J=5.2, 12.4 Hz, 1H), 4.15-3.69 (m, 3H), 3.67-3.52 (m, 4H), 3.35 (s, 3H), 2.98-2.84 (m, 2H), 2.76-2.59 (m, 4H), 2.08-1.97 (m, 1H), 1.40 (s, 9H); LC-MS (ESI+) m/z 458.1 (M+H)⁺.

### Step 2—3-[3-methyl-2-oxo-5-(piperazin-1-ylmethyl)-1,3-benzodiazol-1-yl]piperidine-2,6-dione

To a stirred solution of tert-butyl 4-{[1-(2,6-dioxopiperidin-3-yl)-3-methyl-2-oxo-1,3-benzodiazol-5-yl]methyl}piperazine-1-carboxylate (75 mg, 0.164 mmol) and in DCM (3 mL) was added 4 M HCl (gas) in 1,4-dioxane (1 mL) at rt. The resulting mixture was stirred for 2 h at rt. On completion, the reaction mixture was concentrated under reduced pressure. The residue was purified by trituration with Et₂O (10 mL) to afford the title compound (50 mg) as a white solid. LC/MS (ESI, m/z): [(M+H)]⁺=358.1.

### Example 1.136. Preparation of 3-{3-methyl-2-oxo-4-[2-(piperidin-4-yl)ethynyl]-1,3-benzodiazol-1-yl}piperidine-2,6-dione (Intermediate EF)

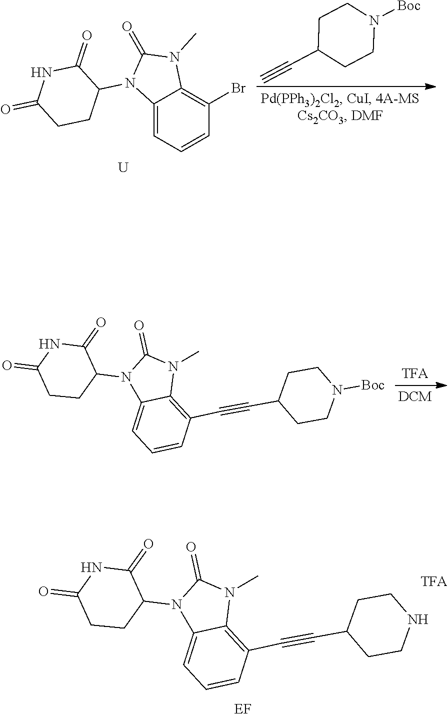

### Step 1—Tert-butyl 4-[2-[1-(2,6-dioxo-3-piperidyl)-3-methyl-2-oxo-benzimidazol-4-yl]ethynyl] piperidine-1-carboxylate

To a solution of 3-(4-bromo-3-methyl-2-oxobenzimidazol-1-yl)piperidine-2,6-dione (400 mg, 1.18 mmol, Intermediate U), tert-butyl 4-ethynylpiperidine-1-carboxylate (446 mg, 2.13 mmol, CAS #287192-97-6) in DMF (5.00 mL) was added Cs₂CO₃ (1.93 g, 5.91 mmol), 4 Å molecular sieves (100 mg), Pd(PPh₃)₂Cl₂ (83.0 mg, 118 umol) and CuI (22.5 mg, 118 umol). The mixture was stirred at 80° C. for 2 hrs under N₂. On completion, the reaction mixture was filtered and the filter cake was washed with ACN (10 mL). The filtrate was concentrated in vacuo to give a residue. The residue was purified by reverse phase (0.1% FA condition) to give the title compound (330 mg, 60% yield) as yellow solid. ¹H NMR (400 MHz, DMSO-d₆) δ 11.11 (s, 1H), 7.13-7.11 (m, 1H), 7.07-7.05 (m, 1H), 7.01-6.97 (m, 1H), 5.40-5.36 (m, 1H), 3.72-3.66 (m, 2H), 3.63 (s, 3H), 3.12-3.07 (m, 2H), 2.93-2.87 (m, 2H), 2.75-2.68 (m, 1H), 2.65-2.60 (m, 1H), 2.04-1.99 (m, 1H), 1.88-1.85 (m, 2H), 1.8-1.50 (m, 2H), 1.40 (s, 9H).

### Step 2—3-{3-Methyl-2-oxo-4-[2-(piperidin-4-yl)ethynyl]-1,3-benzodiazol-1-yl}piperidine-2,6-dione Trifluoroacetate

To a stirred solution of tert-butyl 4-{2-[1-(2,6-dioxopiperidin-3-yl)-3-methyl-2-oxo-1,3-benzodiazol-4-yl]ethynyl}piperidine-1-carboxylate (100 mg, 0.2 mmol) in DCM (4 mL) was added TFA (1 mL) dropwise at rt. The resulting mixture was stirred for 2 h at rt under nitrogen atmosphere. On completion, the reaction mixture was concentrated under reduced pressure. The residue was purified by trituration with Et₂O to give the title compound (90 mg) as a white solid. LC/MS (ESI, m/z): [(M+1)]⁺=367.1.

### Example 1.137. Preparation of 3-(4-(20-amino-3,6,9,12,15,18-hexaoxaicosyl)-3-methyl-2-oxo-2,3-dihydro-1H-benzo[d]imidazol-1-yl)piperidine-2,6-dione (Intermediate EG)

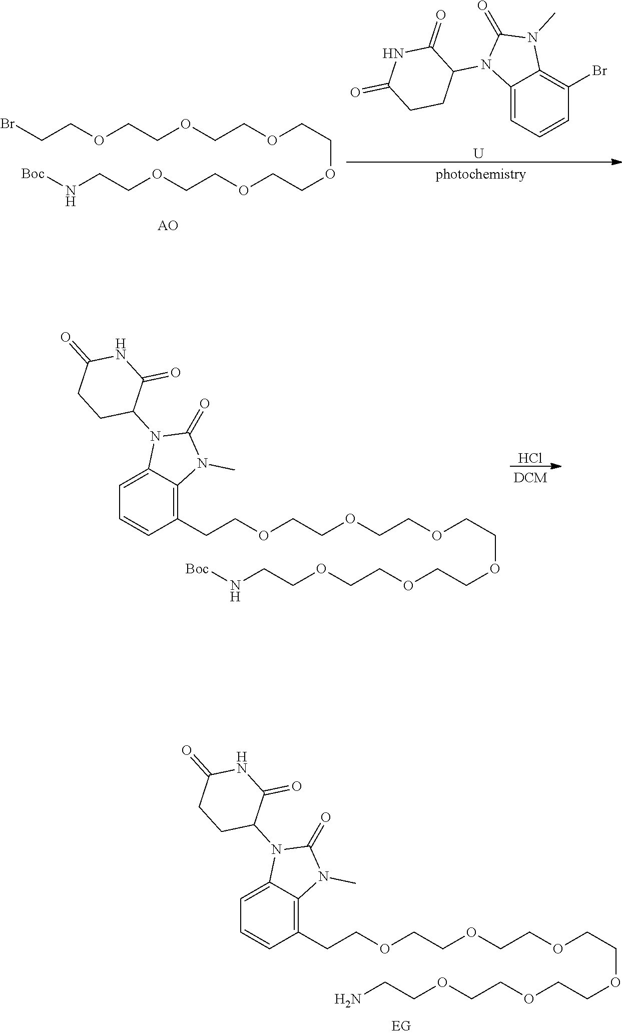

### Step 1—Tert-butyl (20-(1-(2,6-dioxopiperidin-3-yl)-3-methyl-2-oxo-2,3-dihydro-1H-benzo[d] imidazol-5-yl)-3,6,9,12,15,18-hexaoxaicosyl)carbamate

To a solution of 3-(4-bromo-3-methyl-2-oxo-2,3-dihydro-1H-benzo[d]imidazol-1-yl) piperidine-2,6-dione (3 g, 8.87 mmol, Intermediate C) and tert-butyl (20-bromo-3,6,9,12,15,18-hexaoxaicosyl)carbamate (4.71 g, 9.64 mmol, Intermediate AO) in DME (15 mL) was added Ir[dF(CF3)ppy]2(dtbpy)(PF6) (99.53 mg, 88.72 umol), TTMSS (2.21 g, 8.87 mmol, 2.74 mL) and Na₂CO₃ (1.88 g, 17.7 mmol) at 10° C. The reaction was stirred at 25° C. for 16 hr irradiated with a 34 W blue LED lamp (7 cm away). On completion, the mixture was filtered and filter cake was washed with EA (50 mL). The filtrate was concentrated in vacuo to afford a residue. The residue was purified by column chromatography (SiO₂, EA:EtOH=10:1), followed by reversed-phase HPLC (0.1% FA condition) to afford the title compound (3.11 g, 4.57 mmol, 52% yield) as colorless oil. ¹H NMR (400 MHz, CHLOROFORM-d) δ=8.26-8.07 (m, 1H), 7.08-6.85 (m, 2H), 6.70 (d, J=7.6 Hz, 1H), 5.29-4.75 (m, 2H), 3.77-3.71 (m, 5H), 3.69-3.63 (m, 20H), 3.55 (t, J=5.2 Hz, 2H), 3.33 (br t, J=4.8 Hz, 2H), 3.24 (t, J=7.2 Hz, 2H), 3.00-2.75 (m, 3H), 2.29-2.20 (m, 1H), 1.47 (s, 9H)

### Step 2—3-(4-(20-amino-3,6,9,12,15,18-hexaoxaicosyl)-3-methyl-2-oxo-2,3-dihydro-1H-benzo[d]imidazol-1-yl)piperidine-2,6-dione

To a stirred solution of tert-butyl N-{20-[1-(2,6-dioxopiperidin-3-yl)-3-methyl-2-oxo-1,3-benzodiazol-5-yl]-3,6,9,12,15,18-hexaoxaicosan-l-yl}carbamate (150 mg, 0.23 mmol) in DCM (3 mL) was added 4 M HCl (gas) in 1,4-dioxane (1 mL) at rt. The resulting mixture was stirred for 2 h at rt. The resulting mixture was concentrated under reduced pressure to afford the title compound (105 mg) as a white solid. LC/MS (ESI, m/z): [(M+H)]⁺=567.3.

### Example 1.138. Preparation of 3-[4-(9-aminononyl)-3-methyl-2-oxo-1,3-benzodiazol-1-yl]piperidine-2,6-dione (Intermediate EH)

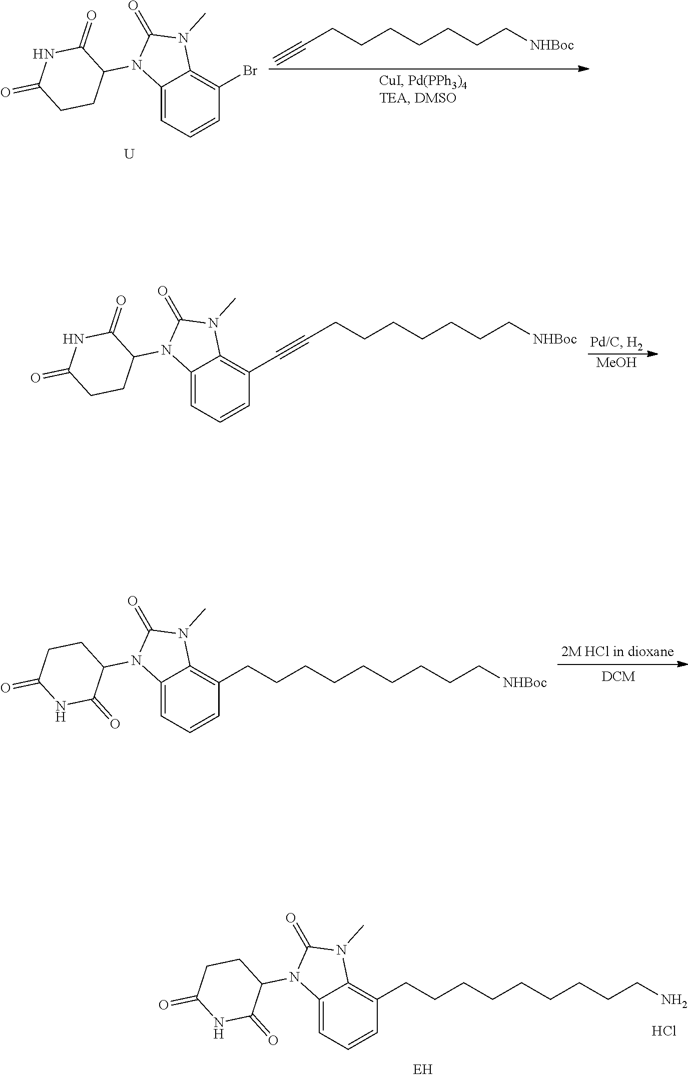

### Step 1—Tert-butyl N-[9-[1-(2,6-dioxopiperidin-3-yl)-3-methyl-2-oxo-1,3-benzodiazol-4-yl]non-8-yn-1-yl]carbamate

To a stirred mixture of 3-(4-bromo-3-methyl-2-oxo-1,3-benzodiazol-1-yl)piperidine-2,6-dione (8.00 g, 23.7 mmol, Intermediate U) and tert-butyl N-(non-8-yn-1-yl)carbamate (8.49 g, 35.5 mmol, CAS #1903797-81-8) in DMA (40.00 mL) and TEA (20.00 mL) were added Pd(PPh₃)₄ (2.73 g, 2.37 mmol) in portions at rt under nitrogen atmosphere. The resulting mixture was stirred for 3 h at 80° C. under nitrogen atmosphere. On completion, the resulting mixture was diluted with water (100 mL). The resulting mixture was extracted with EtOAc (3×75 mL). The combined organic layers were washed with brine (3×50 mL), and dried over anhydrous Na₂SO₄. After filtration, the filtrate was concentrated under reduced pressure. The residue product was purified by reverse phase flash (Column: Spherical C18, 20˜40 um, 330 g; Mobile Phase A: Water (0.05% FA), Mobile Phase B: ACN; Flow rate: 45 mL/min; Gradient (B %): 5%˜5%, 8 min; 40%˜60%, 30 min; 60%˜95%; 0 min; 95%, 5 min; Detector: 254 nm; Rt: 23.4 min.) to afford the title compound (6.4 g, 55% yield) as a yellow solid. ¹H NMR (400 MHz, DMSO-d₆) δ 11.12 (s, 1H), 7.11 (dd, J=7.8, 1.3 Hz, 1H), 7.05 (dd, J=7.9, 1.2 Hz, 1H), 6.99 (t, J=7.8 Hz, 1H), 6.77 (t, J=5.8 Hz, 1H), 5.39 (dd, J=12.7, 5.4 Hz, 1H), 3.64 (s, 3H), 2.98-2.78 (m, 3H), 2.74-2.59 (m, 2H), 2.52-2.46 (m, 2H), 2.06-1.96 (m, 1H), 1.61-1.54 (m, 2H), 1.45-1.34 (m, 13H), 1.33-1.19 (m, 4H). LC/MS (ESI, m/z): [(M+1)]⁺=497.3.

### Step 2—Tert-butyl N-[9-[1-(2,6-dioxopiperidin-3-yl)-3-methyl-2-oxo-1,3-benzodiazol-4-yl]nonyl]carbamate

To a stirred mixture of tert-butyl N-[9-[1-(2,6-dioxopiperidin-3-yl)-3-methyl-2-oxo-1,3-benzodiazol-4-yl]non-8-yn-1-yl]carbamate (6.40 g, 12.9 mmol) in MeOH (500.00 mL) was added Pd/C (1371.48 mg, 12.887 mmol) in portions at rt under nitrogen atmosphere. The resulting mixture was stirred for 4 h at rt under hydrogen atmosphere. On completion, the reaction mixture was filtered. The filter cake was washed with MeOH (3×100 mL). The filtrate was concentrated under reduced pressure to give the title compound (6.0 g) as a white solid. ¹H NMR (400 MHz, DMSO-d₆) δ 11.10 (s, 1H), 7.02-6.91 (m, 2H), 6.88-6.85 (m, 1H), 6.75 (t, J=5.7 Hz, 1H), 5.37 (dd, J=12.6, 5.4 Hz, 1H), 3.55 (s, 3H), 2.90-2.87 (m, 3H), 2.78-2.56 (m, 2H), 2.04-1.94 (m, 1H), 1.61-1.56 (m, 2H), 1.38-1.32 (m, 15H), 1.29-1.19 (m, 8H). LC/MS (ESI, m/z): [(M+1)]⁺=501.3.

### Step 3—3-[4-(9-Aminononyl)-3-methyl-2-oxo-1,3-benzodiazol-1-yl]piperidine-2,6-dione Hydrochloride

To a stirred solution of tert-butyl N-[9-[1-(2,6-dioxopiperidin-3-yl)-3-methyl-2-oxo-1,3-benzodiazol-4-yl]nonyl]carbamate (6.00 g, 12 mmol) in DCM (20.00 mL) was added HCl (4 M) in 1,4-dioxane (20.00 mL) at rt under nitrogen atmosphere. The solution was stirred for 4 h at rt. On completion, the mixture was concentrated under reduced pressure to give the title compound (3.9 g, 91% yield) as a white solid. ¹H NMR (400 MHz, DMSO-d₆) δ 11.11 (s, 1H), 8.03 (broad, 3H), 7.03-6.91 (m, 2H), 6.89-6.86 (m, 1H), 5.40 (dd, J=12.6, 5.4 Hz, 1H), 3.57 (s, 3H), 2.94-2.85 (m, 3H), 2.81-2.57 (m, 4H), 2.03-1.97 (m, 1H), 1.64-1.53 (m, 4H), 1.41-1.26 (m, 10H). LC/MS (ESI, m/z): [(M+1)]⁺=401.3.

### Example 1.139. Preparation of 3-(4-{2-[2-(2-aminoethoxy)ethoxy]ethyl}-3-methyl-2-oxo-1,3-benzodiazol-1-yl)piperidine-2,6-dione (Intermediate EI)

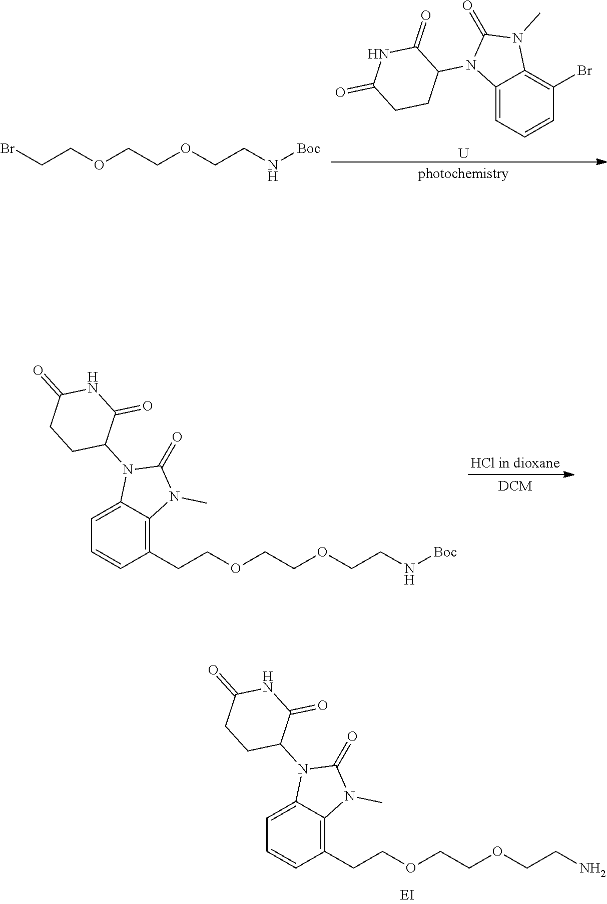

### Step 1—Tert-butyl N-[2-[2-[2-[1-(2,6-dioxo-3-piperidyl)-3-methyl-2-oxo-benzimidazol-4-yl]ethoxy]ethoxy]ethyl]carbamate

To a solution of 3-(4-bromo-3-methyl-2-oxo-benzimidazol-1-yl)piperidine-2,6-dione (6.00 g, 17.7 mmol, Intermediate U) in DME (170 mL) was added Ir[dF(CF3)ppy]₂(dtbpy)(PF₆) (199 mg, 177 umol), tert-butyl N-[2-[2-(2-bromoethoxy)ethoxy]ethyl]carbamate (7.20 g, 23.0 mmol, CAS #165963-71-3) Na₂CO₃ (3.76 g, 35.4 mmol) TTMSS (4.41 g, 17.7 mmol, 5.47 mL) and NiCl₂.dtbbpy (35.3 mg, 88.7 umol) at 25° C. The vial was sealed and placed under nitrogen atmosphere. The reaction was stirred and irradiated with a 34 W blue LED lamp (7 cm away), with cooling fan to keep the reaction temperature at 25° C. for 14 hr. On completion, the reaction mixture was diluted with (PE/EA=3/1) (300 mL), filtered and the solid dried under reduced pressure to give the crude product. Then the solid was purified by prep-HPLC (FA condition) to give the title compound (4.22 g, 85% yield) as white solid. ¹H NMR (400 MHz, CDCl₃-d) δ=8.14 (br d, J=1.2 Hz, 1H), 7.07-6.99 (m, 1H), 6.98-6.93 (m, 1H), 6.71 (d, J=7.6 Hz, 1H), 5.29-5.18 (m, 1H), 4.96 (br s, 1H), 3.81-3.69 (m, 5H), 3.61 (s, 4H), 3.51 (t, J=5.2 Hz, 1H), 3.56-3.46 (m, 1H), 3.35-3.19 (m, 4H), 3.01-2.70 (m, 3H), 2.29-2.18 (m, 1H), 1.47 (s, 9H); LC-MS (ESI⁺) m/z 391.1 (M+H)⁺.

### Step 2—3-(4-{2-[2-(2-aminoethoxy)ethoxy]ethyl}-3-methyl-2-oxo-1,3-benzodiazol-1-yl)piperidine-2,6-dione Hydrochloride

To a stirred solution of tert-butyl N-[2-(2-{2-[1-(2,6-dioxopiperidin-3-yl)-3-methyl-2-oxo-1,3-benzodiazol-4-yl]ethoxy}ethoxy)ethyl]carbamate (150 mg, 0.31 mmol) in DCM (3 mL) was added 4 M HCl (gas) in 1,4-dioxane (1 mL) at rt. The resulting mixture was stirred for 2 h at rt. On completion, the reaction mixture was concentrated under reduced pressure. The residue was triturated with Et₂O to afford the title compound (110 mg) as a white solid. LC/MS (ESI, m/z): [(M+H)]⁺=391.2.

### Example 1.140. Preparation of 3-[4-(14-amino-3,6,9,12-tetraoxatetradecan-1-yl)-3-methyl-2-oxo-1,3-benzodiazol-1-yl]piperidine-2,6-dione (Intermediate EJ)

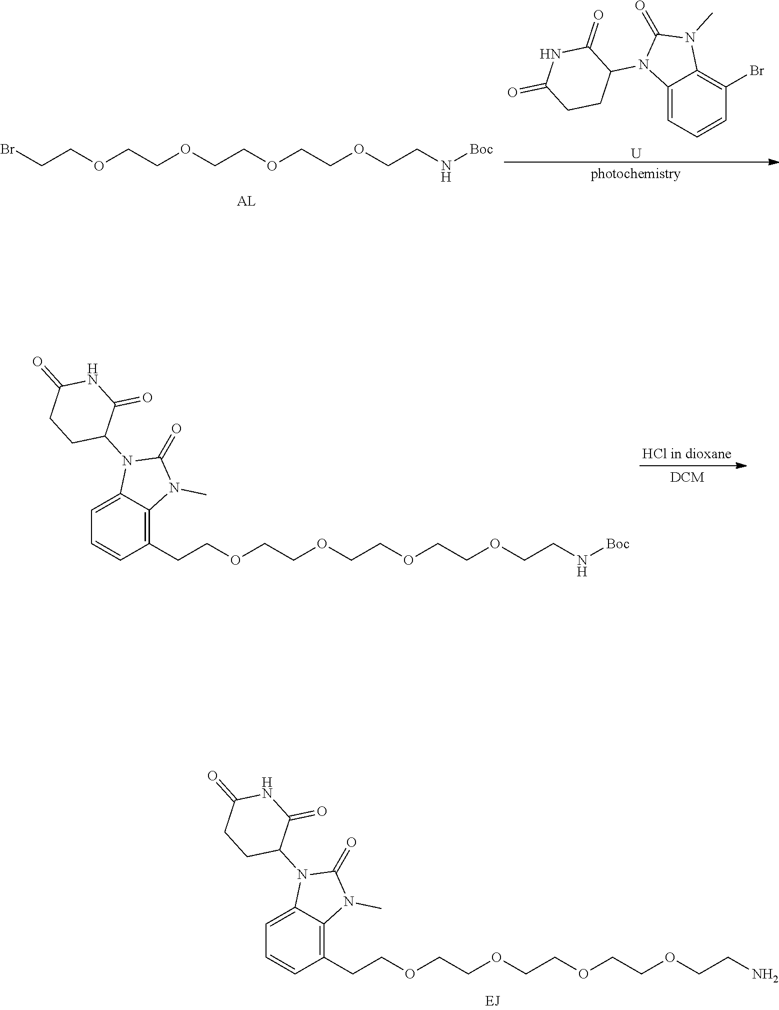

### Step 1—Tert-butyl (14-(1-(2,6-dioxopiperidin-3-yl)-3-methyl-2-oxo-2,3-dihydro1H-benzo[d]imidazol-4-yl)-3,6,9,12-tetraoxatetradecyl)carbamate

To a solution of tert-butyl N-[2-[2-[2-[2-(2-bromoethoxy)ethoxy]ethoxy]ethoxy]ethyl]carbamate (6.16 g, 15.4 mmol, Intermediate AL) and 3-(4-bromo-3-methyl-2-oxo-benzimidazol-1-yl)piperidine-2,6-dione (4 g, 10 mmol, Intermediate U) in DME (100 mL) was added TTMSS (2.94 g, 11.83 mmol, 3.65 mL), Na₂CO₃ (2.51 g, 23.7 mmol), bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (132.71 mg, 118.29 umol) and 4-tert-butyl-2-(4-tert-butyl-2-pyridyl)pyridine; dichloronickel (235.39 mg, 591.44 umol) under N₂. The reaction was stirred and irradiated with a 34 W blue LED lamp (7 cm away), with cooling fan to keep the reaction temperature at 25° C. for 14 hr. On completion, the reaction was filtered to remove the insoluble materials and the filtrate was concentrated in vacuo. The residue was purified by column chromatography (SiO₂, PE:EA=1:1 to 0:1, P: Rf=0.1), followed by reversed-phase HPLC (0.1% FA condition) to give the title compound (2.77 g, 39% yield) as white solid. ¹H NMR (400 MHz, CHLOROFORM-d) δ=8.45 (br s, 1H), 7.04-6.97 (m, 1H), 6.96-6.90 (m, 1H), 6.70 (dd, J=0.8, 7.8 Hz, 1H), 5.23 (dd, J=5.3, 12.4 Hz, 1H), 5.07 (br s, 1H), 3.75 (t, J=7.2 Hz, 2H), 3.71 (s, 3H), 3.67-3.59 (m, 12H), 3.54 (t, J=5.2 Hz, 2H), 3.32 (br s, 2H), 3.23 (t, J=7.2 Hz, 2H), 2.99-2.68 (m, 3H), 2.29-2.15 (m, 1H), 1.46 (s, 9H).

### Step 2—3-[4-(14-amino-3,6,9,12-tetraoxatetradecan-1-yl)-3-methyl-2-oxo-1,3-benzodiazol-1-yl]piperidine-2,6-dione Hydrochloride

To a stirred solution of tert-butyl N-{14-[1-(2,6-dioxopiperidin-3-yl)-3-methyl-2-oxo-1,3-benzodiazol-4-yl]-3,6,9,12-tetraoxatetradecan-1-yl}carbamate (150 mg, 0.26 mmol) in DCM (3 mL) was added 4 M HCl (gas) in 1,4-dioxane (1 mL) at rt. The resulting mixture was stirred for 1 h at rt. On completion, the reaction mixture was concentrated under reduced pressure. The residue was triturated with Et₂O to afford the title compound (120 mg) as a white solid. LC/MS (ESI, m/z): [(M+H)]⁺=479.3.

### Example 1.141. Preparation of Tert-butyl 3-[3-[1-(2,6-dioxo-3-piperidyl)-3-methyl-2-oxo-benzimidazol-5-yl]propoxy]propanoate (Intermediate EK)

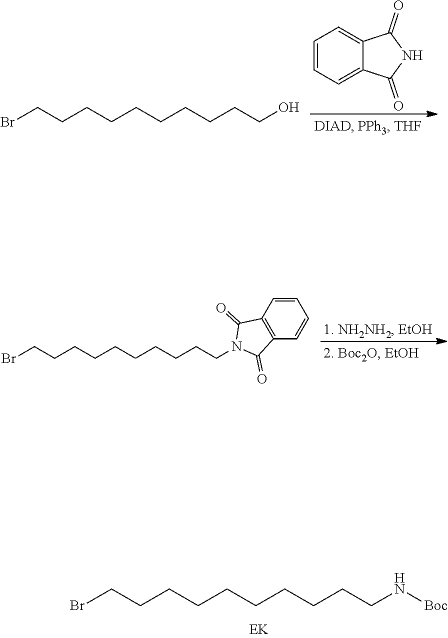 [SMILES: O=C1NC(=O)c2ccccc21 ; OCCCCCCCCCCBr ; O=C1c2ccccc2C(=O)N1CCCCCCCCCCBr ; CC(C)(C)OC(=O)NCCCCCCCCCCBr]

### Step 1—Tert-butyl 3-prop-2-ynoxypropanoate

To a solution of 10-bromodecan-1-ol (20 g, 84.33 mmol, CAS #53463-68-6) and isoindoline-1,3-dione (14.89 g, 101.19 mmol) in THF (250 mL) was added PPh₃ (33.18 g, 126.5 mmol) and DIAD (25.58 g, 126.49 mmol, 24.59 mL) at 0° C. The mixture was stirred at 20° C. for 15 h. On completion, the reaction mixture was filtered and the filtrate was concentrated to give oil. The resulting oil was purified by silica gel column chromatography (PE:EA=10:1-5:1) to give the title compound (22.4 g, 73% yield) as white solid. ¹H NMR (400 MHz, CDCl₃) δ 7.93-7.83 (m, 2H), 7.79-7.68 (m, 2H), 3.80-3.60 (m, 2H), 3.42 (t, J=6.8 Hz, 2H), 1.86 (quin, J=7.2 Hz, 2H), 1.69 (quin, J=7.2 Hz, 2H), 1.49-1.17 (m, 12H).

### Step 2—Tert-butyl 3-[3-[1-(2,6-dioxo-3-piperidyl)-3-methyl-2-oxo-benzimidazol-5-yl]propoxy] propanoate

To a solution of 2-(10-bromodecyl)isoindoline-1,3-dione (15 g, 41 mmol) in EtOH (150 mL) was added NH₂NH₂·H₂O (4.18 g, 81.9 mmol, 4.06 mL) at 20° C. The mixture was stirred at 80° C. for 1.5 h. On completion, the reaction was cooled to rt and a white solid participated. The mixture was filtered, and the filter cake was washed with EtOH (100 mL). Then a solution of Boc₂O (80.44 g, 368.56 mmol, 84.67 mL) in EtOH (150 mL) was added to the above filtrate. The mixture was stirred at 20° C. for 15 h. On completion, the mixture was filtered, and the filtrate was concentrated under reduced pressure to give a residue. The residue was purified by silica gel column chromatography (Petroleum ether/Ethyl acetate=100/1, 10/1) to give the title compound (6.0 g, 44% yield) as light yellow solid. ¹H NMR (400 MHz, CDCl₃) δ 4.50 (br s, 1H), 3.54-3.36 (m, 2H), 3.11 (br d, J=6.4 Hz, 2H), 1.86 (quin, J=7.2 Hz, 2H), 1.59-1.11 (m, 23H). MS (ESI⁺) m/z 236.1, 238.1 (M+1-100)⁺.

### Example 1.142. Preparation of 3-(4-(10-aminodecyl)-3-methyl-2-oxo-2,3-dihydro-1H-benzo[d]imidazol-1-yl)piperidine-2,6-dione (Intermediate EL)

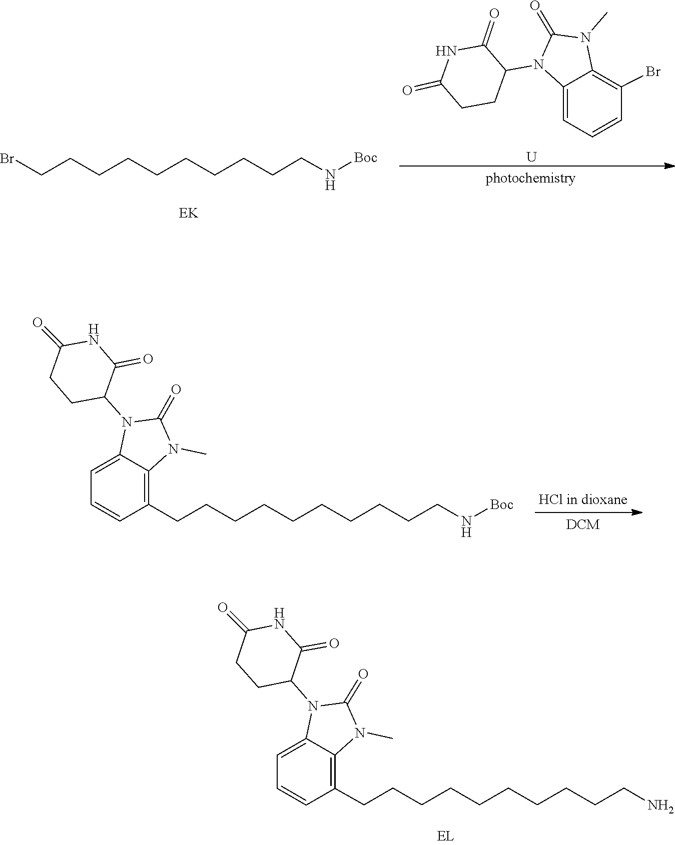

### Step 1—3-[3-[1-(2,6-Dioxo-3-piperidyl)-3-methyl-2-oxo-benzimidazol-5-yl]propoxy]propanoic Acid

To an vial equipped with a stir bar was added 3-(4-bromo-3-methyl-2-oxo-benzimidazol-1-yl)piperidine-2,6-dione (5.0 g, 15 mmol, Intermediate U), tert-butyl N-(10-bromodecyl)carbamate (6.22 g, 18.5 mmol, Intermediate EK), bis(trimethylsilyl)silyl-trimethyl-silane (3.68 g, 14.8 mmol, 4.56 mL), bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (1.66 g, 1.48 mmol), NiCl₂.dtbbpy (29.42 mg, 73.93 umol), Na₂CO₃ (3.13 g, 29.6 mmol) and DME (150 mL). The vial was sealed and placed with nitrogen. The reaction was stirred and irradiated with a 34 W blue LED lamp (7 cm away), with cooling fan to keep the reaction temperature at 25° C. for 15 hr. On completion, the reaction mixture was filtered and the filtrate concentrated to give oil. The resulting oil was purified by silica gel column chromatography (PE:EA=100:1-10:1) to give the crude product. The crude product was then purified by reversed-phase HPLC (0.1% FA condition) to give the title compound (2.75 g, 35% yield) as white solid. ¹H NMR (400 MHz, CDCl₃) δ 8.23-8.05 (m, 1H), 7.04-6.95 (m, 1H), 6.94-6.85 (m, 1H), 6.68 (d, J=7.4 Hz, 1H), 5.33-5.14 (m, 1H), 4.52 (br s, 1H), 3.69 (s, 3H), 3.12 (br d, J=5.2 Hz, 2H), 3.03-2.69 (m, 5H), 2.29-2.18 (m, 1H), 1.76-1.58 (m, 3H), 1.55-1.19 (m, 21H), LC-MS (ESI⁺) m/z 515.2 (M+1)⁺.

### Step 2—3-(4-(10-Aminodecyl)-3-methyl-2-oxo-2,3-dihydro-1H-benzo[d]imidazol-1-yl)piperidine-2,6-dione Hydrochloride

To a stirred mixture of tert-butyl (10-(1-(2,6-dioxopiperidin-3-yl)-3-methyl-2-oxo-2,3-dihydro-1H-benzo[d]imidazol-4-yl)decyl)carbamate (100.00 mg, 0.194 mmol) in DCM (1 mL) was added 4 M HCl (gas) in 1,4-dioxane (0.3 mL) dropwise at rt. The resulting mixture was stirred for 1 h at rt under nitrogen atmosphere. On completion, the reaction mixture was concentrated under reduced pressure. The residue was triturated with Et₂O to afford the title compound (80 mg) as a white solid. LC/MS (ESI, m/z): [(M+H)]⁺=415.3.

### Example 1.143. Preparation of 2-[(3-{[(1R,2S)-2-fluorocyclopropyl]carbamoyl}-8-(methylamino)imidazo[1,2-b]pyridazin-6-yl)amino]benzoic Acid (Intermediate EM)

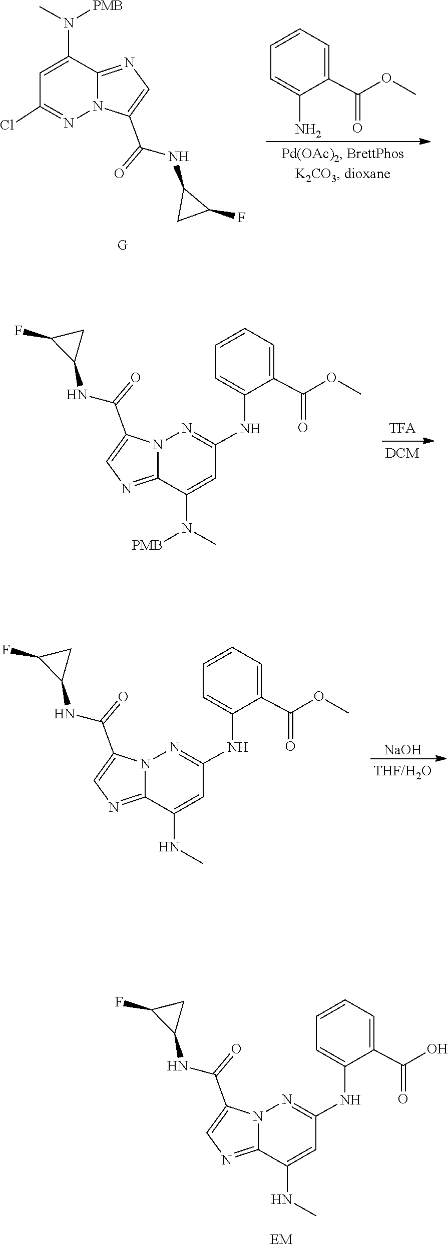

### Step 1—Methyl 2-[(3-{[(1R,2S)-2-fluorocyclopropyl]carbamoyl}-8-{[(4-methoxyphenyl)methyl](methyl)amino}imidazo[1,2-b]pyridazin-6-yl)amino]benzoate

To a stirred solution of 6-chloro-N-[(1R,2S)-2-fluorocyclopropyl]-8-{[(4-methoxyphenyl)methyl](methyl)amino}imidazo[1,2-b]pyridazine-3-carboxamide (2 g, 4.952 mmol, Intermediate G) and methyl anthranilate (0.75 g, 4.952 mmol) in dioxane (40 mL) were added K₂CO₃ (1.37 g, 9.90 mmol), BrettPhos (0.27 g, 0.50 mmol) and Pd(OAc)₂ (0.11 g, 0.50 mmol) in turns at rt under nitrogen atmosphere. The resulting mixture was stirred for 16 h at 80° C. under nitrogen atmosphere. On completion, the reaction mixture was cooled to rt and filtered. The filtrate was concentrated under reduced pressure. The residue was purified by silica gel column chromatography, eluted with Petroleum ether/EtAO (5:1), to afford the title compound (1.98 g, 77% yield) as a brown solid. LC/MS (ESI, m/z): [(M+H)]⁺=519.2.

### Step 2—Methyl 2-[(3-{[(1R,2S)-2-fluorocyclopropyl]carbamoyl}-8-(methylamino)imidazo[1,2-b]pyridazin-6-yl)amino]benzoate

To a solution of methyl 2-[(3-{[(1R,2S)-2-fluorocyclopropyl]carbamoyl}-8-{[(4-methoxyphenyl)methyl](methyl)amino}imidazo[1,2-b]pyridazin-6-yl)amino]benzoate (1.90 g, 3.66 mmol) in DCM (20 ml) was added TFA (10 ml) dropwise at 0° C. The reaction mixture was stirred for 2 h at rt under nitrogen atmosphere. On completion, the reaction mixture was concentrated under reduced pressure. The residue was purified by trituration with Et₂O (20 mL). The solid was dried under reduced pressure to afford the title compound (1.10 g, 75%) as a white solid. LC/MS (ESI, m/z): [(M+H)]⁺=399.2.

### Step 3—2-[(3-{[(1R,2S)-2-fluorocyclopropyl]carbamoyl}-8-(methylamino)imidazo[1,2-b]pyridazin-6-yl)amino]benzoic Acid

To a solution of 2-[(3-{[(1R,2S)-2-fluorocyclopropyl]carbamoyl}-8-(methylamino)imidazo[1,2-b]pyridazin-6-yl)amino]benzoate (1.10 g, 2.76 mmol) in THF (10 mL) was added 1N aq. NaOH (10 mL) dropwise at rt. The reaction mixture was stirred for 1 h at rt under nitrogen atmosphere. On completion, the reaction mixture was concentrated under reduced pressure. The residue was purified by reverse phase flash chromatography (Column: WelFlash™ C18-I, 20-40 μm, 330 g; Eluent A: Water (plus 10 mmol/L FA); Eluent B: ACN; Gradient: 30%-60% B in 30 min; Flow rate: 80 mL/min; Detector: 220/254 nm; desired fractions were collected at 43% B) and concentrated under reduced pressure to afford the title compound (500 mg, 47% yield) as a yellow solid. LC/MS (ESI, m/z): [(M+H)]⁺=385.1.

### Example 1.144. Preparation of 2-{[5-(ethylcarbamoyl)-4-{[3-(5-fluoropyrimidin-2-yl)-2-methoxyphenyl]amino}pyridin-2-yl]amino}pyridine-4-carboxylic Acid (Intermediate EN)

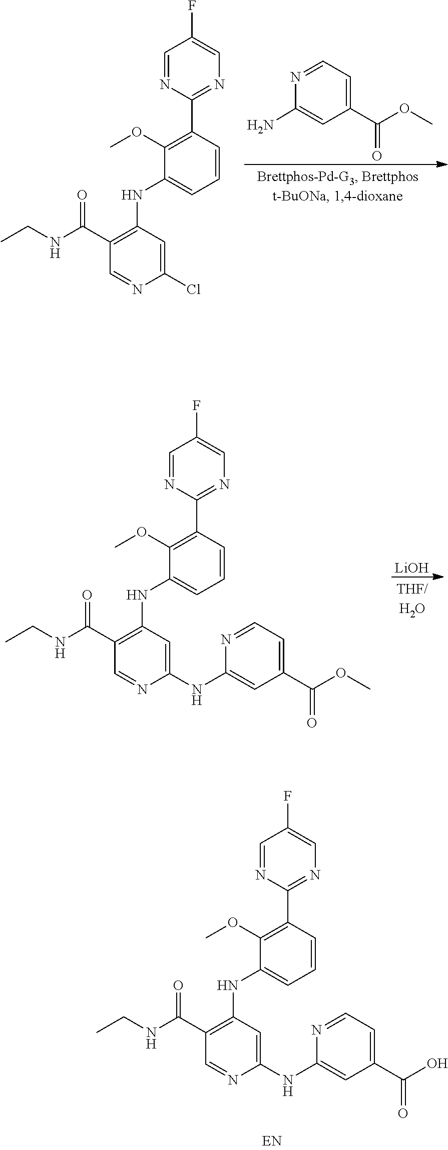

### Step 1—Methyl 2-{[5-(ethylcarbamoyl)-4-{[3-(5-fluoropyrimidin-2-yl)-2-methoxyphenyl]amino}pyridin-2-yl]amino}pyridine-4-carboxylate

To a stirred mixture of 6-chloro-N-ethyl-4-{[3-(5-fluoropyrimidin-2-yl)-2-methoxyphenyl]amino}pyridine-3-carboxamide (200 mg, 0.5 mmol, synthesized via Steps 1-2 of Intermediate X) and methyl 2-aminopyridine-4-carboxylate (0.3 g, 2 mmol) in dioxane (5 mL) were added BrettPhos Pd G3 (0.05 g, 0.050 mmol) and BrettPhos (0.03 g, 0.050 mmol) and t-BuONa (0.09 g, 1 mmol) at rt under nitrogen atmosphere. The resulting mixture was stirred for 16 h at 130° C. under nitrogen atmosphere. On completion, the reaction mixture was cooled to rt and precipitated solids were collected by filtration and washed with dioxane (2×6 mL). The residue was purified by reverse phase flash (Column: WelFlash™ C18-I, 20-40 um, 120 g; Eluent A: Water (10 mmol/L NH₄HCO₃); Eluent B: ACN; Gradient: 25%-55% B in 25 min; Flow rate: 60 mL/min; Detector: 254 nm; desired fractions were collected at 35% B) and concentrated under reduced pressure to afford the title compound (130 mg, 51% yield) as a white solid. LC/MS (ESI, m/z): [(M+H)]⁺=518.2.

### Step 2—2-{[5-(Ethylcarbamoyl)-4-{[3-(5-fluoropyrimidin-2-yl)-2-methoxyphenyl]amino}pyridin-2-yl]amino}pyridine-4-carboxylic Acid

To a stirred mixture of methyl 2-{[5-(ethylcarbamoyl)-4-{[3-(5-fluoropyrimidin-2-yl)-2-methoxyphenyl]amino}pyridin-2-yl]amino}pyridine-4-carboxylate (0.13 g, 0.25 mmol) and LiOH (0.06 g, 2.5 mmol) in THF (5 mL) and H₂O (5 mL) at rt under nitrogen atmosphere. The resulting mixture was stirred for 2 h at rt under nitrogen atmosphere. On completion, the reaction mixture was basified to pH 3 with HCl (aq.). The precipitated solids were collected by filtration and washed with H₂O (2×6 mL). The solid was dried under vacuum to afford the title compound (0.09 g, 71% yield) as a white solid. LC/MS (ESI, m/z): [(M+H)]⁺=504.1.

### Example 1.145. Preparation of 3-[3-methyl-2-oxo-5-(piperidin-3-yl)-1,3-benzodiazol-1-yl]piperidine-2,6-dione (Intermediate EO)

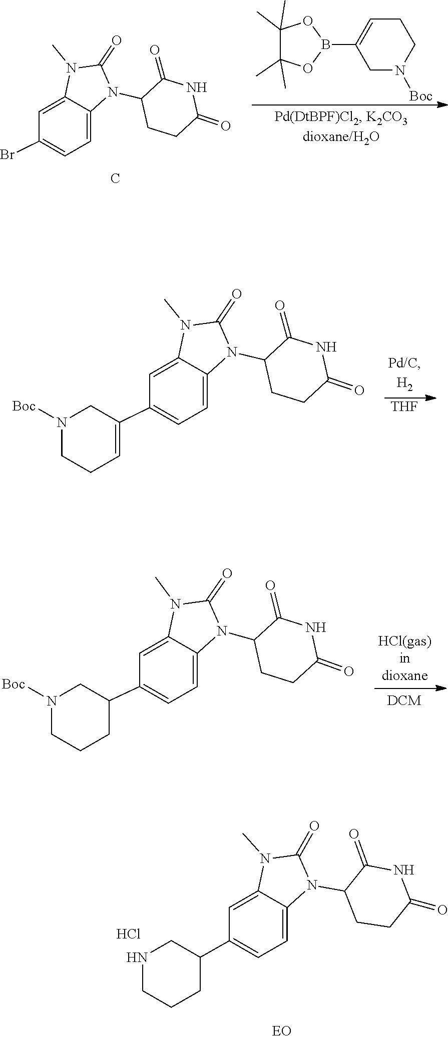

### Step 1—Tert-butyl 3-[1-(2,6-dioxopiperidin-3-yl)-3-methyl-2-oxo-1,3-benzodiazol-5-yl]-5,6-dihydro-2H-pyridine-1-carboxylate

To a stirred solution of 3-(5-bromo-3-methyl-2-oxo-1,3-benzodiazol-1-yl)piperidine-2,6-dione (500 mg, 2 mmol, Intermediate C) and tert-butyl 3-(4,4,5,5-tetramethyl-1,3,2-dioxaborolan-2-yl)-5,6-dihydro-2H-pyridine-1-carboxylate (548.63 mg, 1.775 mmol) in dioxane (5 mL) and H₂O (1 mL) were added Pd(DtBPF)Cl₂ (96.37 mg, 0.148 mmol) and K₂CO₃ (408.70 mg, 2.958 mmol) at rt. The resulting mixture was stirred for 1 h at 90° C. under nitrogen atmosphere. On completion, the mixture was cooled to rt and was acidified to pH 5 with 1 N HCl(aq). The resulting mixture was filtered, and the filter cake was washed with acetonitrile (3×50 mL). The filtrate was concentrated under reduced pressure. The residue was purified by reverse flash chromatography (column, C18 silica gel; mobile phase, MeCN in Water (0.1% FA), 30% to 60% gradient in 30 min; detector, UV 254 nm. Flow rate: 80 mL/min; Detector: 254 nm; desired fractions were collected at 46% B) and concentrated under reduced pressure to afford the title compound (480 mg, 74% yield) as a brown yellow solid. LC/MS (ESI, m/z): [(M+H)]⁺=441.2.

### Step 2—Tert-butyl 4-[1-(2,6-dioxopiperidin-3-yl)-3-methyl-2-oxo-1,3-benzodiazol-4-yl]piperidine-1-carboxylate

To a solution of 3-[1-(2,6-dioxopiperidin-3-yl)-3-methyl-2-oxo-1,3-benzodiazol-5-yl]-5,6-dihydro-2H-pyridine-1-carboxylate (480 mg, 1.09 mmol) in THF (3 mL) was added Pd/C (10 wt %, 50 mg) under nitrogen atmosphere. The reaction system was degassed under vacuum and purged with H₂ several time, then it was hydrogenated under H₂ balloon (1 atm) at rt for 16 h. After completion of the reaction, Pd/C was filtered off through celite and the corresponding filtrate was concentrated under reduced pressure to afford the title compound (420 mg, 87% yield) as a white oil. LC/MS (ESI, m/z): [(M+H)]⁺=443.2.

### Step 3—3-[3-Methyl-2-oxo-5-(piperidin-3-yl)-1,3-benzodiazol-1-yl]piperidine-2,6-dione Hydrochloride

To a stirred solution of tert-butyl 3-[1-(2,6-dioxopiperidin-3-yl)-3-methyl-2-oxo-1,3-benzodiazol-5-yl]piperidine-1-carboxylate (420 mg, 0.949 mmol) in DCM (5 mL) was added 4 M HCl (gas) in 1,4-dioxane (1 mL) dropwise at rt. The resulting mixture was stirred for 2 h at rt under nitrogen atmosphere. On completion, the reaction mixture was concentrated under reduced pressure. The residue was purified by trituration with ethyl ether (50 mL) to afford the title compound (300 mg, 92% yield) as a white solid. LC/MS (ESI, m/z): [(M+H)]⁺=343.2.

### Example 1.146. Preparation of 3-[3-methyl-2-oxo-4-(piperidin-4-yl)-1,3-benzodiazol-1-yl]piperidine-2,6-dione (Intermediate EP)

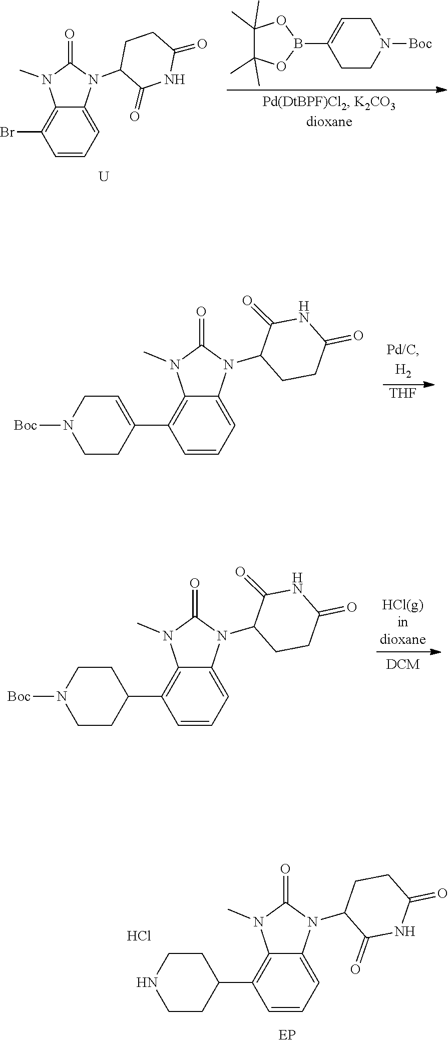

### Step 1—Tert-butyl 4-[1-(2,6-dioxopiperidin-3-yl)-3-methyl-2-oxo-1,3-benzodiazol-4-yl]-3,6-dihydro-2H-pyridine-1-carboxylate

To a stirred solution of 3-(4-bromo-3-methyl-2-oxo-1,3-benzodiazol-1-yl)piperidine-2,6-dione (500 mg, 2 mmol, Intermediate U) and tert-butyl 4-(4,4,5,5-tetramethyl-1,3,2-dioxaborolan-2-yl)-3,6-dihydro-2H-pyridine-1-carboxylate (548.63 mg, 1.775 mmol) in dioxane (5 mL) and H₂O (1 mL) were added Pd(DtBPF)Cl₂ (96.37 mg, 0.148 mmol) and K₂CO₃ (408.70 mg, 2.958 mmol) at rt. The resulting mixture was stirred for 1 h at 90° C. under nitrogen atmosphere. On completion, the mixture was cooled to rt and was acidified to pH 5 with 1 N HCl (aq). The resulting mixture was filtered, and the filter cake was washed with acetonitrile (3×30 mL). The filtrate was concentrated under reduced pressure. The residue was purified by reverse flash chromatography (column, C18 silica gel; mobile phase, MeCN in Water (0.1% FA), 30% to 60% gradient in 30 min; detector, UV 254 nm. Flow rate: 80 mL/min; Detector: 254 nm; desired fractions were collected at 46% B) and concentrated under reduced pressure to afford the title compound (300 mg, 46% yield) as a yellow solid. LC/MS (ESI, m/z): [(M+H)]⁺=441.3.

### Step 2—Tert-butyl 4-[1-(2,6-dioxopiperidin-3-yl)-3-methyl-2-oxo-1,3-benzodiazol-4-yl]piperidine-1-carboxylate

To a solution of 4-[1-(2,6-dioxopiperidin-3-yl)-3-methyl-2-oxo-1,3-benzodiazol-4-yl]-3,6-dihydro-2H-pyridine-1-carboxylate (280 mg, 0.636 mmol) in THF (3 mL) was added Pd/C (10 wt %, 30 mg) under nitrogen atmosphere. The reaction system was degassed under vacuum and purged with H₂ several times, then was hydrogenated under H₂ balloon (1 atm) at for rt 16 h. After completion of the reaction, Pd/C was filtered off through celite and the corresponding filtrate was concentrated under reduced pressure to afford the title compound (250 mg, 89% yield) as a yellow solid. LC/MS (ESI, m/z): [(M+H)]⁺=443.1.

### Step 3—3-[3-Methyl-2-oxo-4-(piperidin-4-yl)-1,3-benzodiazol-1-yl]piperidine-2,6-dione Hydrochloride

To a stirred solution of tert-butyl 4-[1-(2,6-dioxopiperidin-3-yl)-3-methyl-2-oxo-1,3-benzodiazol-5-yl]piperidine-1-carboxylate (140 mg, 0.32 mmol) in DCM (5 mL) was added 4 M HCl (gas) in 1,4-dioxane (1 mL) dropwise at rt. The resulting mixture was stirred for 1 h at rt under nitrogen atmosphere. On completion, the reaction mixture was concentrated under reduced pressure. The residue was purified by trituration with ethyl ether (10 mL) to afford the title compound (100 mg, 92%) as a yellow solid. LC/MS (ESI, m/z): [(M+H)]⁺=343.1.

### Example 1.147. Preparation of 3-[3-methyl-2-oxo-5-(piperidin-4-yl)-1,3-benzodiazol-1-yl]piperidine-2,6-dione (Intermediate EQ)

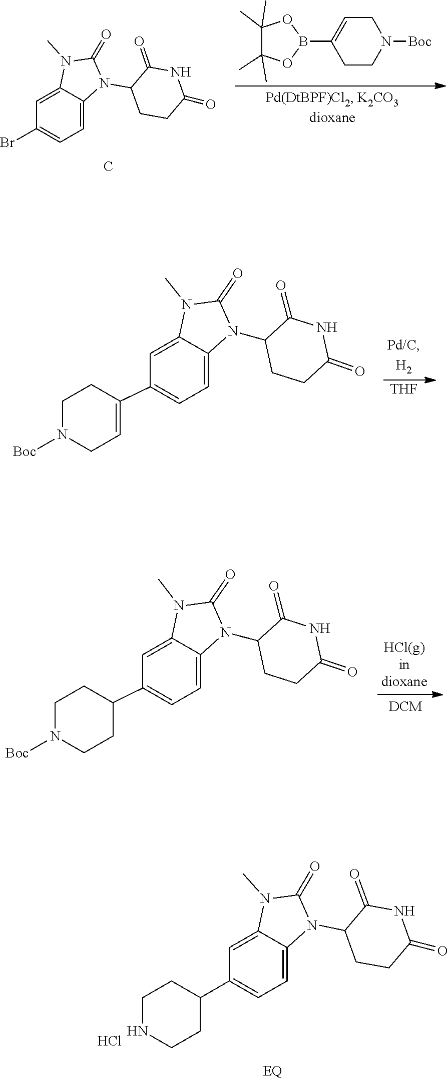

### Step 1—Tert-butyl 4-[1-(2,6-dioxopiperidin-3-yl)-3-methyl-2-oxo-1,3-benzodiazol-5-yl]-3,6-dihydro-2H-pyridine-1-carboxylate

To a stirred solution of 3-(5-bromo-3-methyl-2-oxo-1,3-benzodiazol-1-yl)piperidine-2,6-dione (500 mg, 2 mmol, Intermediate C) and tert-butyl 4-(4,4,5,5-tetramethyl-1,3,2-dioxaborolan-2-yl)-3,6-dihydro-2H-pyridine-1-carboxylate (548.63 mg, 1.775 mmol) in dioxane (5 mL) and H₂O (1 mL) were added Pd(DtBPF)Cl₂ (96.37 mg, 0.148 mmol) and K₂CO₃ (408.70 mg, 2.958 mmol) at rt. The resulting mixture was stirred for 1 h at 90° C. under nitrogen atmosphere. On completion, the reaction was cooled to rt and was acidified to pH 5 with 1 N HCl(aq). The resulting mixture was filtered, and the filter cake was washed with acetonitrile (3×30 mL). The filtrate was concentrated under reduced pressure. The residue was purified by reverse flash chromatography (column, C18 silica gel; mobile phase, MeCN in Water (0.1% FA), 30% to 60% gradient in 30 min; detector, UV 254 nm. Flow rate: 80 mL/min; Detector: 254 nm; desired fractions were collected at 42% B) and concentrated under reduced pressure to afford the title compound (150 mg, 23% yield) as a yellow solid. LC/MS (ESI, m/z): [(M+H)]⁺=441.2.

### Step 2—Tert-butyl 4-[1-(2,6-dioxopiperidin-3-yl)-3-methyl-2-oxo-1,3-benzodiazol-5-yl]piperidine-1-carboxylate

To a solution of tert-butyl 4-[1-(2,6-dioxopiperidin-3-yl)-3-methyl-2-oxo-1,3-benzodiazol-5-yl]-3,6-dihydro-2H-pyridine-1-carboxylate (150 mg, 0.34 mmol) in THF (3 mL) was added Pd/C (10 wt %, 20 mg) under nitrogen atmosphere. The reaction system was degassed under vacuum and purged with H₂ several times, then it was hydrogenated under H₂ balloon (1 atm) at rt for 16 h. After completion of the reaction, Pd/C was filtered off through celite and the corresponding filtrate was concentrated under reduced pressure to afford the title compound (140 mg, 93% yield) as a yellow solid. LC/MS (ESI, m/z): [(M+H)]⁺=443.2.

### Step 3—3-[3-Methyl-2-oxo-5-(piperidin-4-yl)-1,3-benzodiazol-1-yl]piperidine-2,6-dione Hydrochloride

To a stirred solution of tert-butyl 4-[1-(2,6-dioxopiperidin-3-yl)-3-methyl-2-oxo-1,3-benzodiazol-5-yl]piperidine-1-carboxylate (140 mg, 0.32 mmol) and HCl (gas) in DCM (5 mL) was added in 4 M 1,4-dioxane (1 mL) at rt under nitrogen atmosphere. The resulting mixture was stirred for 1 h at at under nitrogen atmosphere. On completion, the resulting mixture was concentrated under reduced pressure. The residue was purified by trituration with ethyl ether (10 mL) to afford the title compound (100 mg, 92% yield) as a yellow solid. LC/MS (ESI, m/z): [(M+H)]⁺=343.2.

### Example 1.148. Preparation of 3-{3-methyl-4-[3-(methylamino)pyrrolidin-1-yl]-2-oxo-1,3-benzodiazol-1-yl}piperidine-2,6-dione (Intermediate ER)

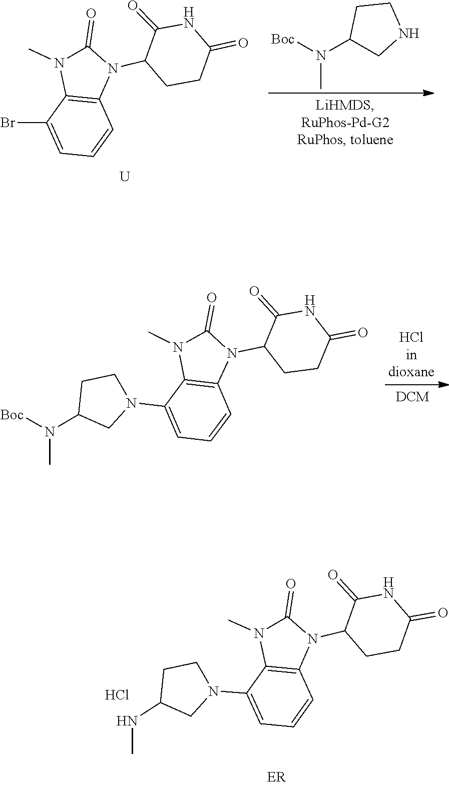

### Step 1—Tert-butyl 3-{[1-(2,6-dioxopiperidin-3-yl)-3-methyl-2-oxo-1,3-benzodiazol-4-yl](methyl)amino}pyrrolidine-1-carboxylate

To a stirred mixture of 3-(4-bromo-3-methyl-2-oxo-1,3-benzodiazol-1-yl)piperidine-2,6-dione (0.60 g, 1.8 mmol, Intermediate U) and tert-butyl 3-(methylamino)pyrrolidine-1-carboxylate (0.71 g, 3.548 mmol, CAS #172478-00-1) in methylbenzene (12 mL) were added RuPhos-PdCl-2nd G (0.27 g, 0.355 mmol), RuPhos (0.16 g, 0.355 mmol) and LiHMDS (10.65 mL, 10.644 mmol) at rt under nitrogen atmosphere. The resulting mixture was stirred for 2 h at 80° C. under nitrogen atmosphere. On completion, the reaction was cooled to rt and was neutralized to pH 4 with FA. The precipitated solids were collected by filtration and washed with ACN (3×7 mL). The resulting mixture was concentrated under reduced pressure. The residue was purified by reverse phase flash (Column: WelFlash™ C18-I, 20-40 um, 120 g; Eluent A: Water (plus 10 mmol/L FA); Eluent B: ACN; Gradient: 25%-55% B in 25 min; Flow rate: 60 mL/min; Detector: 254 nm; desired fractions were collected at 50% B) and concentrated under reduced pressure to afford the title compound (0.36 g, 44% yield) as a yellow solid. LC/MS (ESI, m/z): [(M+H)]⁺=458.2.

### Step 2—3-{3-Methyl-4-[3-(methylamino)pyrrolidin-1-yl]-2-oxo-1,3-benzodiazol-1-yl}piperidine-2,6-dione Hydrochloride

To a stirred mixture of tert-butyl 3-{3-methyl-4-[3-(methylamino)pyrrolidin-1-yl]-2-oxo-1,3-benzodiazol-1-yl}-2,6-dioxopiperidine-1-carboxylate (0.36 g, 0.79 mmol) in DCM (3 mL) was added 4 M HCl (gas) in 1,4-dioxane (1 ml) dropwise at rt. The resulting mixture was stirred for 2 h at rt. On completion, the reaction mixture was concentrated under reduced pressure. The residue was triturated with Et₂O to afford the title compound (0.30 g, 97% yield) as a pink solid. LC/MS (ESI, m/z): [(M+H)]⁺=358.1.

### Example 1.149. Preparation of 3-{3-Methyl-5-[methyl(piperidin-4-yl)amino]-2-oxo-1,3-benzodiazol-1-yl}piperidine-2,6-dione (Intermediate ES)

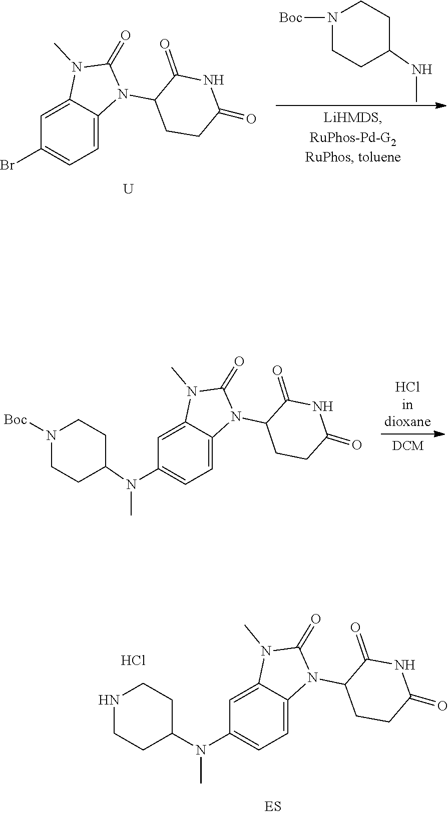

### Step 1—Tert-butyl 4-{[1-(2,6-dioxopiperidin-3-yl)-3-methyl-2-oxo-1,3-benzodiazol-5-yl](methyl)amino}piperidine-1-carboxylate

To a stirred mixture of 3-(5-bromo-3-methyl-2-oxo-1,3-benzodiazol-1-yl)piperidine-2,6-dione (0.50 g, 2 mmol, Intermediate U) and tert-butyl 4-(methylamino)piperidine-1-carboxylate (0.63 g, 3.0 mmol) in methylbenzene (10 mL) were added RuPhos-PdCl-2nd G (0.23 g, 0.30 mmol), RuPhos (0.12 g, 0.30 mmol) and LiHMDS (8.87 mL, 8.87 mmol) at rt under nitrogen atmosphere. The resulting mixture was stirred for 2 h at 80° C. under nitrogen atmosphere. On completion, the mixture was cooled to rt and was neutralized to pH 4 with FA. The resulting mixture was filtered, and the filter cake was washed with DCM (3×10 mL). The filtrate was concentrated under reduced pressure. The residue was purified by reverse phase flash (Column: WelFlash™ C18-I, 20-40 um, 120 g; Eluent A: Water (plus 10 mmol/L FA); Eluent B: ACN; Gradient: 25%-55% B in 25 min; Flow rate: 60 mL/min; Detector: 254 nm; desired fractions were collected at 42% B) and concentrated under reduced pressure to afford the title compound (0.50 g, 72% yield) as a brown oil. LC/MS (ESI, m/z): [(M+H)]⁺=472.2.

### Step 2—3-{3-Methyl-5-[methyl(piperidin-4-yl)amino]-2-oxo-1,3-benzodiazol-1-yl}piperidine-2,6-dione Hydrochloride

To a stirred mixture of tert-butyl 4-{[1-(2,6-dioxopiperidin-3-yl)-3-methyl-2-oxo-1,3-benzodiazol-5-yl](methyl)amino}piperidine-1-carboxylate (0.50 g, 1.1 mmol) in DCM (5 mL) was added HCl (gas) in 1,4-dioxane (5 mL) at rt. The resulting mixture was stirred for 1 h at rt. On completion, the reaction mixture was concentrated under reduced pressure. The residue was triturated with Et₂O to afford the title compound (0.38 g) as a yellow solid. LC/MS (ESI, m/z): [(M+H)]⁺=372.2.

### Example 1.150. Preparation of 3-{3-methyl-5-[methyl(pyrrolidin-3-yl)amino]-2-oxo-1,3-benzodiazol-1-yl}piperidine-2,6-dione (Intermediate ET)

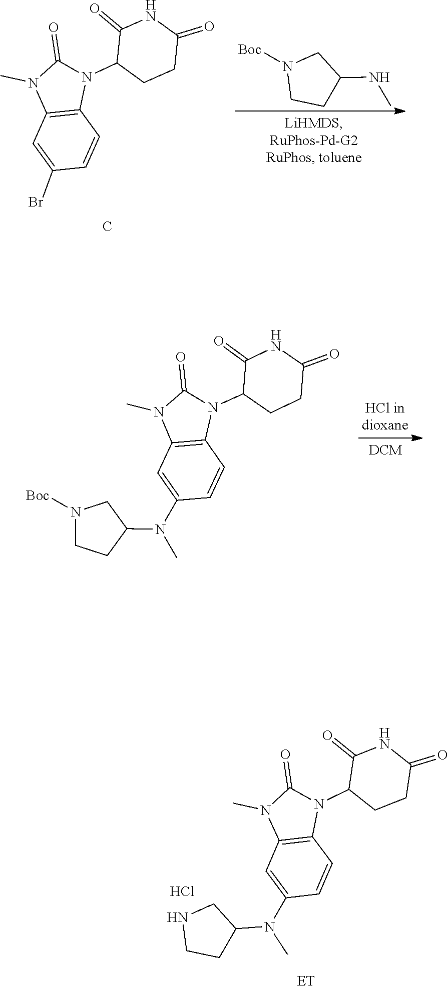

### Step 1—Tert-butyl 3-{[1-(2,6-dioxopiperidin-3-yl)-3-methyl-2-oxo-1,3-benzodiazol-5-yl](methyl)amino}pyrrolidine-1-carboxylate

To a stirred mixture of tert-butyl 3-(methylamino)pyrrolidine-1-carboxylate (592.27 mg, 2.957 mmol) and 3-(5-bromo-3-methyl-2-oxo-1,3-benzodiazol-1-yl)piperidine-2,6-dione (500 mg, 2 mmol, Intermediate C) in toluene (10 mL) were added RuPhos-PdCl-2nd G (229.99 mg, 0.296 mmol) and RuPhos (138.00 mg, 0.296 mmol), followed by added LiHMDS (8.87 mL, 8.874 mmol) at rt under nitrogen atmosphere. The resulting mixture was stirred for 2 h at 80° C. under nitrogen atmosphere. On completion, the mixture was acidified to pH 3 with FA. The resulting mixture was filtered, and the filter cake was washed with MeCN (3×30 mL). The filtrate was concentrated under reduced pressure. The residue was purified by reverse phase flash chromatography (Column: WelFlash™ C18-I, 20-40 um, 330 g; Eluent A: Water (plus 10 mmol/L FA); Eluent B: ACN; Gradient: 25%-60% B in 25 min; Flow rate: 80 mL/min; Detector: 220/254 nm; desired fractions were collected at 45% B) and concentrated under reduced pressure to afford the title compound (380 mg, 56% yield) as a brown solid. LC/MS (ESI, m/z): [(M+H)]⁺=458.3.

### Step 2—3-{3-Methyl-5-[methyl(pyrrolidin-3-yl)amino]-2-oxo-1,3-benzodiazol-1-yl}piperidine-2,6-dione Hydrochloride

To a stirred mixture of tert-butyl 3-{[1-(2,6-dioxopiperidin-3-yl)-3-methyl-2-oxo-1,3-benzodiazol-5-yl](methyl)amino}pyrrolidine-1-carboxylate (380 mg, 0.83 mmol) in DCM (6 mL) was added 4 M HCl (gas) in 1,4-dioxane (2 mL) dropwise at rt. The resulting mixture was stirred for 2 h at rt. On completion, the reaction mixture was concentrated under reduced pressure. The residue was purified by trituration with Et₂O (15 mL) to afford the title compound (320 mg) as a white solid. LC/MS (ESI, m/z): [(M+H)]⁺=358.2.

### Example 1.151. Preparation of tert-butyl (3-(3-bromocyclobutoxy)propyl) (methyl)carbamate (Intermediate EU)

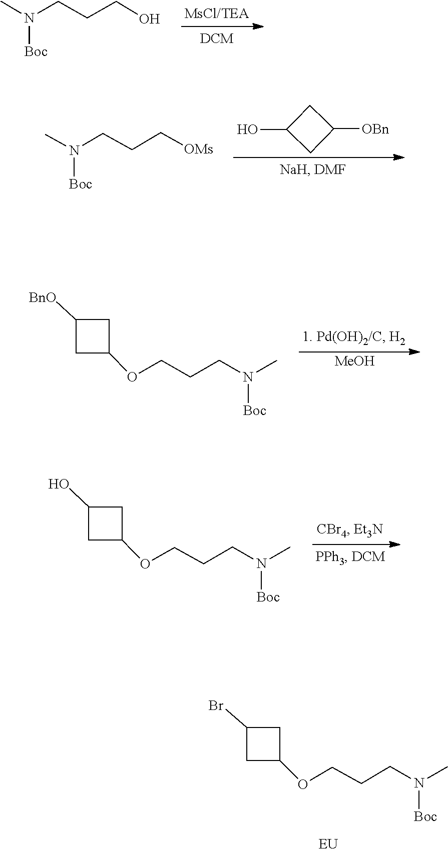

### Step 1—3-[Tert-butoxycarbonyl(methyl)amino]propyl Methanesulfonate

To a solution of tert-butyl N-(3-hydroxypropyl)-N-methyl-carbamate (11.6 g, 61.3 mmol) and Et₃N (8.06 g, 79.7 mmol, 11.09 mL) in DCM (150 mL) was added MsCl (8.12 g, 70.9 mmol, 5.49 mL) at 0° C. The mixture was stirred at 20° C. for 2 h. On completion, the reaction mixture was added to ice-water (200 mL), and extracted with DCM (150 mL×3). The combined organic phase was washed with brine (125 mL×3), dried with anhydrous Na₂SO₄, filtered and concentrated in vacuo to give the title compound (16.4 g) as yellow oil. ¹H NMR (400 MHz, CDCl₃) δ=4.27-4.24 (t, J=6.4 Hz, 2H), 3.37-3.34 (t, J=6.8 Hz, 2H), 3.03 (s, 3H), 2.87 (s, 3H), 2.02-1.95 (q, J=6.4 Hz, 2H), 1.46 (s, 9H).

### Step 2—Tert-butyl N-[3-(3-benzyloxycyclobutoxy)propyl]-N-methyl-carbamate

To a solution of 3-benzyloxycyclobutanol (14.2 g, 79.7 mmol) in DMF (160 mL) was added NaH (3.68 g, 92.0 mmol, 60% dispersion in mineral oil) at 0° C. slowly. The mixture was stirred at 20° C. for 0.5 h, and a solution of 3-[tert-butoxycarbonyl(methyl)amino]propylmethanesulfonate (16.4 g, 61.3 mmol) in DMF (160 mL) was added to above mixture at 20° C. The mixture was stirred at 20° C. for 15 h. On completion, the reaction mixture was added to ice-water (600 mL), and extracted with ethyl acetate (400 mL×3). The combined organic phase was washed with brine (150 mL×4), dried with anhydrous Na2SO4, filtered and concentrated in vacuo. The residue was purified by column chromatography (SiO₂, Petroleum ether/Ethyl acetate=100/1 to 1/5) to give the title compound (15.68 g) as yellow oil. ¹H NMR (400 MHz, CDCl₃) δ=7.27-7.19 (m, 5H), 4.35-4.34 (s, 2H), 3.60-3.48 (m, 2H), 3.27-3.20 (m, 4H), 2.78 (s, 3H), 2.57-2.54 (m, 2H), 1.88-1.85 (m, 2H), 1.71-1.68 (q, J=6.4 Hz, 2H), 1.38 (s, 9H).

### Step 3—Tert-butyl (3-(3-hydroxycyclobutoxy)propyl)(methyl)carbamate

To a solution of tert-butyl N-[3-(3-benzyloxycyclobutoxy)propyl]-N-methyl-carbamate (15.4 g, 44.1 mmol) in MeOH (500 mL) was added Pd/C (3.50 g, 10 wt %) and Pd(OH)₂ (3.5 g, 4.98 mmol, 20 wt %) under N₂ atmosphere. The suspension was degassed and purged with H₂ three times. The mixture was stirred under H₂ (50 psi) at 25° C. for 18 h. On completion, the mixture was filtered and concentrated in vacuo to give the title compound (11 g, 42.42 mmol, 96.25% yield) as a colorless oil. ¹H NMR (400 MHz, CDCl₃) δ=3.93-3.91 (m, 1H), 3.52 (m, 1H), 3.35-3.28 (m, 4H), 2.85 (s, 3H), 2.73-2.70 (m, 2H), 1.89-1.75 (m, 4H), 1.46 (s, 9H).

### Step 4—Tert-butyl (3-(3-bromocyclobutoxy)propyl)(methyl)carbamate

A solution of tert-butyl N-[3-(3-hydroxycyclobutoxy)propyl]-N-methyl-carbamate (9.9 g, 38.17 mmol) in toluene (80 mL) was heated to 110° C. for 0.5 h. A mixture of CBr₄ (30.76 g, 92.76 mmol) in DCM (60 mL) was added to above mixture, and Et₃N (8.77 g, 86.65 mmol, 12.06 mL) was added to the above mixture. Then, a solution of PPh₃ (23.63 g, 90.09 mmol) in DCM (70 mL) was added dropwise at 5-10° C. The reaction was heated to 45° C. for 1.5 hrs then stirred at 25° C. for 13 hrs. On completion, the reaction was diluted with EA (30 mL) and PE (40 mL), and filtered. The filtrate was concentrated in vacuo to give a residue. The residue was purified by column chromatography (SiO₂, Petroleum ether/Ethyl acetate=100/1 to 1/1) to give the title compound (10 g, 81% yield) as yellow oil. ¹H NMR (400 MHz, CDCl₃) δ=4.53-4.48 (q, J=5.9 Hz, 1H), 4.38-4.34 (q, J=6.1 Hz, 1H), 3.35-3.27 (m, 4H), 2.85 (s, 3H), 2.66-2.63 (t, J=6.0 Hz, 4H), 1.81-1.74 (q, J=6.7 Hz, 2H), 1.46 (s, 9H).

### Example 1.152. Preparation of 3-(3-methyl-5-{3-[3-(methylamino)propoxy]cyclobutyl}-2-oxo-1,3-benzodiazol-1-yl)piperidine-2,6-dione (Intermediate EV)

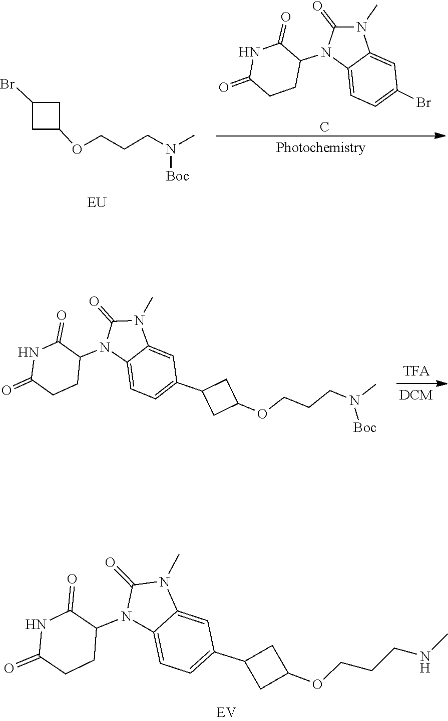

### Step 1—Tert-butyl (3-(3-(1-(2,6-dioxopiperidin-3-yl)-3-methyl-2-oxo-2,3-dihydro-1H-benzo[d]imidazol-5-yl)cyclobutoxy)propyl)(methyl)carbamate

To a solution of tert-butyl N-[3-(3-bromocyclobutoxy)propyl]-N-methyl-carbamate (4.17 g, 12.9 mmol, Intermediate EU), 3-(5-bromo-3-methyl-2-oxo-benzimidazol-1-yl)piperidine-2,6-dione (3.50 g, 10.4 mmol, Intermediate C), 4-tert-butyl-2-(4-tert-butyl-2-pyridyl)pyridine; dichloronickel (205.97 mg, 517.51 umol), bis[3,5-difluoro-2-[5-(trifluoromethyl)-2-pyridyl]phenyl]iridium(1+); 4-tert-butyl-2-(4-tert-butyl-2-pyridyl)pyridine; hexafluorophosphate (116 mg, 104 umol) and Na₂CO₃ (2.19 g, 20.7 mmol) in DME (100 mL) was added TTMSS (2.57 g, 10.35 mmol, 3.19 mL) under N₂. The reaction was stirred and irradiated with a 34 W blue LED lamp (7 cm away), with cooling fan to keep the reaction temperature at 25° C. for 14 hr. On completion, the reaction mixture was filtered and the filtrate was concentrated in vacuo. The residue was purified by column chromatography (SiO₂, PE:EA=3:1 to 0:1, TLC (PE:EA=0:1, P: Rf=0.5)) to give the crude product. Then the product was further purified by reversed-phase HPLC (0.1% FA condition) to give the title compound (3.36 g, 65% yield) as white solid). ¹H NMR (400 MHz, CDCl₃) δ=8.42-8.41 (br s, 1H), 6.96-6.89 (m, 2H), 6.76-6.72 (t, J=7.9 Hz, 1H), 5.25-5.20 (m, 1H), 4.22-4.4.13 (m, 1H), 3.95-3.93 (m, 0.5H), 3.44-3.31 (m, 7H), 2.87 (m, 0.5H), 2.72-2.70 (m, 5H), 2.60-2.35 (m, 2H), 2.05-2.01 (m, 2H), 1.84-1.79 (m, 2H), 1.75 (m, 2H), 1.46 (s, 9H).

### Step 2—3-(3-methyl-5-{3-[3-(methylamino)propoxy]cyclobutyl}-2-oxo-1,3-benzodiazol-1-yl)piperidine-2,6-dione Trifluoroacetate

To a stirred mixture of tert-butyl N-(3-{3-[1-(2,6-dioxopiperidin-3-yl)-3-methyl-2-oxo-1,3-benzodiazol-5-yl]cyclobutoxy}propyl)-N-methylcarbamate (75 mg, 0.15 mmol) in DCM (2 mL) was added TFA (1 ml) at rt. The resulting mixture was stirred for 1 h at rt. On completion, the reaction mixture was concentrated under reduced pressure. The residue was purified by trituration with Et₂O (5 mL) to afford the title compound (75 mg) as a white oil. LC/MS (ESI, m/z): [(M+H)]⁺=401.2.

### Example 1.153. Preparation of Tert-butyl 2-(bromomethyl)-7-azaspiro[3.5]nonane-7-carboxylate (Intermediate EW)

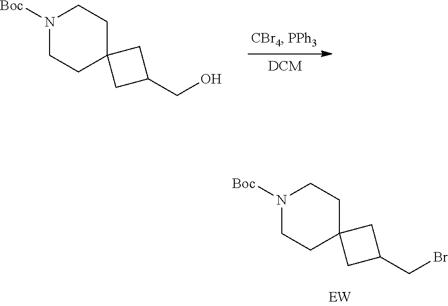 [SMILES: CC(C)(C)OC(=O)N1CCC2(CC1)CC(CO)C2 ; CC(C)(C)OC(=O)N1CCC2(CC1)CC(CBr)C2]

To a solution of tert-butyl 2-(hydroxymethyl)-7-azaspiro[3.5]nonane-7-carboxylate (4.00 g, 15.6 mmol, CAS #1356476-27-1) in DCM (150 mL) was added PPh₃ (12.3 g, 47.0 mmol) and CBr₄ (15.6 g, 46.9 mmol) at 0° C. and stirred at 25° C. for 2 hr. On completion, the mixture was filtered and the filtrate was concentrated in vacuo. The residue was purified by column chromatography (SiO₂, PE:EA=20:1 to 10:1, P: Rf=0.7 (PE:EA=5:1)) to give the title compound (4.3 g, 86% yield) as white solid. ¹H NMR (400 MHz, CDCl₃-d) δ=3.44 (d, J=7.4 Hz, 2H), 3.39-3.32 (m, 2H), 3.30-3.24 (m, 2H), 2.72-2.59 (m, 1H), 2.04-1.96 (m, 2H), 1.59-1.47 (m, 6H), 1.45 (s, 9H).

### Example 1.154. Preparation of 3-(5-{7-azaspiro[3.5]nonan-2-ylmethyl}-3-methyl-2-oxo-1,3-benzodiazol-1-yl)piperidine-2,6-dione (Intermediate EX)

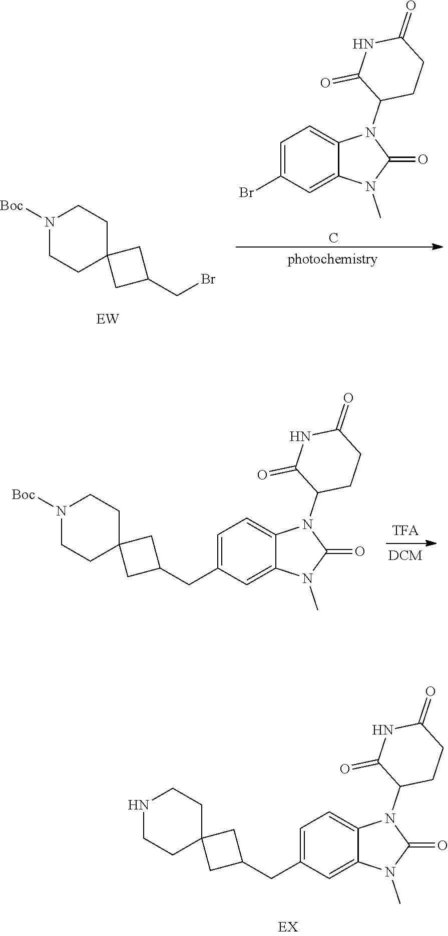

### Step 1—Tert-butyl 3-((1-(2,6-dioxopiperidin-3-yl)-3-methyl-2-oxo-2,3-dihydro-1H-benzo[d]imidazol-5-yl)methyl)-3-methylazetidine-1-carboxylate

Tert-butyl 2-(bromomethyl)-7-azaspiro[3.5]nonane-7-carboxylate (3.39 g, 10.7 mmol, Intermediate EW), 3-(5-bromo-3-methyl-2-oxo-benzimidazol-1-yl) piperidine-2, 6-dione (3.00 g, 8.87 mmol, Intermediate C), Ir[dF (CF₃) ppy]2 (dtbpy) (PF₆) (99.5 mg, 88.7 μmol), NiCl₂. Dtbbpy (17.65 mg, 44.36 μmol), TTMSS (2.21 g, 8.87 mmol, 2.74 mL) and Na₂CO₃ (1.88 g, 17.7 mmol) was dissolved in DME (100 mL). The vial was sealed and placed with nitrogen gas. The reaction was stirred and irradiated with a 34 W blue LED lamp (7 cm away), with cooling fan to keep the reaction temperature at 25° C. for 14 hr. On completion, the reaction mixture was filtered and filter concentrated under reduced pressure to give a residue. The crude product was purified by reversed-phase HPLC (0.1% FA) to give the crude product (3.2 g, 86% purity). The crude product was triturated with EA (20 mL) at 25° C. for 10 min and filtered, then washed EA (10×2 mL). The filter cake was dried under reduced pressure to give the title compound (2.18 g, 49% yield) as white solid. ¹H NMR (400 MHz, DMSO-d₆) δ=11.08 (s, 1H), 7.05-6.93 (m, 2H), 6.83 (d, J=8.4 Hz, 1H), 5.32 (dd, J=5.6, 12.8 Hz, 1H), 3.31 (s, 3H), 3.26-3.10 (m, 4H), 2.97-2.82 (m, 1H), 2.74-2.56 (m, 4H), 2.49-2.41 (m, 1H), 2.04-1.95 (m, 1H), 1.92-1.82 (m, 2H), 1.54-1.42 (m, 4H), 1.41 (br d, J=5.6 Hz, 2H), 1.37 (s, 9H).

### Step 2—3-(5-{7-Azaspiro[3.5]nonan-2-ylmethyl}-3-methyl-2-oxo-1,3-benzodiazol-1-yl)piperidine-2,6-dione Trifluoroacetate

To a stirred mixture of tert-butyl 2-{[1-(2,6-dioxopiperidin-3-yl)-3-methyl-2-oxo-1,3-benzodiazol-5-yl]methyl}-7-azaspiro[3.5]nonane-7-carboxylate (75 mg, 0.151 mmol) in DCM (2 mL) was added TFA (0.4 mL) dropwise at rt under nitrogen atmosphere. The resulting mixture was stirred for 1 h at rt under nitrogen atmosphere. On completion, the reaction mixture was concentrated under reduced pressure. The residue was purified by trituration with Et₂O (10 mL) to afford the title compound (74 mg) as a white solid. LC/MS (ESI, m/z): [(M+H)]⁺=397.2.

### Example 1.155. Preparation of Tert-butyl 4-(3-bromopropanoyl)piperazine-1-carboxylate (Intermediate EY)

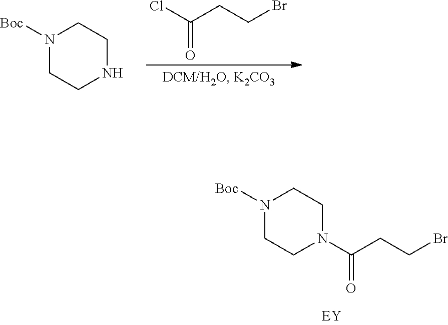 [SMILES: CC(C)(C)OC(=O)N1CCNCC1 ; O=C(Cl)CCBr ; CC(C)(C)OC(=O)N1CCN(C(=O)CCBr)CC1]

To a solution of tert-butyl piperazine-1-carboxylate (5 g, 26.9 mmol) in DCM (200 mL) at 0° C. was added a solution of potassium carbonate (4.08 g, 29.5 mmol) in water (100 mL). Then a solution of 3-bromopropanoyl chloride (9.20 g, 53.7 mmol) in dichloromethane (50 mL) was added to the reaction mixture at 0° C. dropwise and the mixture was stirred at 0-25° C. for 12 hrs. The reaction mixture was extracted with dichloromethane (100 mL×2). The combined organic layers were washed with brine (300 mL×2), dried over anhydrous sodium sulfate, filtered and concentrated under reduced pressure to give a residue. The residue was purified by column chromatography (SiO₂, Petroleum ether/Ethyl acetate=100/1 to 0/1) to give the title compound (6 g, 69% yield) as a white solid. ¹H NMR (400 MHz, CDCl₃-d) δ=3.68 (t, J=7.2 Hz, 2H), 3.66-3.61 (m, 2H), 3.53-3.42 (m, 6H), 2.95 (t, J=7.2 Hz, 2H), 1.49 (s, 9H).

### Example 1.156. Preparation of 3-{3-methyl-2-oxo-5-[3-oxo-3-(piperazin-1-yl)propyl]-1,3-benzodiazol-1-yl}piperidine-2,6-dione (Intermediate EZ)

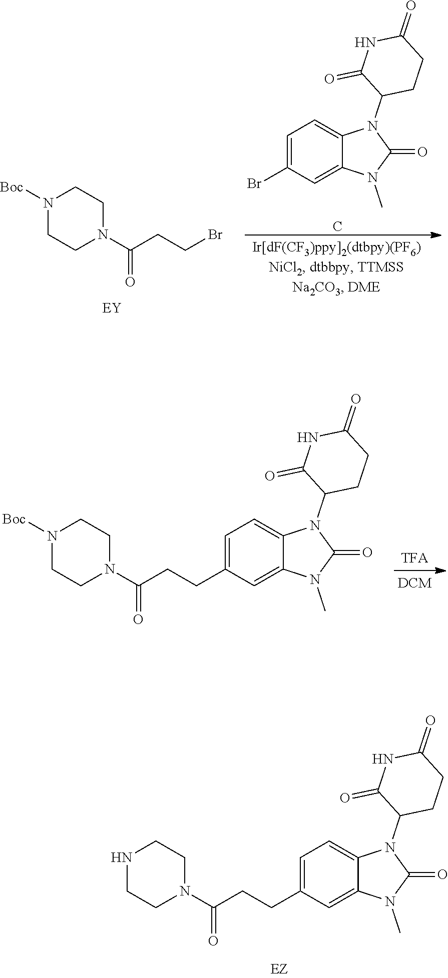

### Step 1—Tert-butyl 4-(3-(1-(2,6-dioxopiperidin-3-yl)-3-methyl-2-oxo-2,3-dihydro-1H-benzo[d]imidazol-5-yl)propanoyl)piperazine-1-carboxylate

To a stir bar was added 3-(5-bromo-3-methyl-2-oxo-benzimidazol-1-yl)piperidine-2,6-dione (4.4 g, 13.0 mmol, Intermediate C), tert-butyl 4-(3-bromopropanoyl)piperazine-1-carboxylate (5.43 g, 16.9 mmol, Intermediate EY), Ir[dF(CF3)ppy]2(dtbpy)(PF6) (146 mg, 130.1 umol), NiCl2.dtbbpy (25.9 mg, 65.1 umol), TTMSS (3.24 g, 13.0 mmol), sodium carbonate (2.76 g, 26.0 mmol) in dimethoxyethane (120 mL). The vial was sealed and placed under nitrogen was added. The reaction was stirred and irradiated with a 34 W blue LED lamp (7 cm away), with cooling fan to keep the reaction temperature at 25° C. for 14 hours. On completion, the reaction mixture was poured into water (100 mL), then filtered and the residue was concentrated under reduced pressure to give a crude product. The crude product was triturated with ethyl acetate (30 mL) at 25° C. for 30 min to give the title compound (2.12 g, 30% yield) as a white solid. ¹H NMR (400 MHz, DMSO-d₆) δ=11.08 (s, 1H), 7.18-6.79 (m, 3H), 5.43-5.21 (m, 1H), 3.55-3.41 (m, 6H), 3.30-3.24 (m, 5H), 2.85 (s, 3H), 2.65 (s, 4H), 2.00 (d, J=6.4 Hz, 1H), 1.41 (s, 9H); LC-MS (ESI+) m/z 522.0 (M+Na)⁺.

### Step 2—3-{3-Methyl-2-oxo-5-[3-oxo-3-(piperazin-1-yl)propyl]-1,3-benzodiazol-1-yl}piperidine-2,6-dione Trifluoroacetate

To a stirred mixture of tert-butyl 4-{3-[1-(2,6-dioxopiperidin-3-yl)-3-methyl-2-oxo-1,3-benzodiazol-5-yl]propanoyl}piperazine-1-carboxylate (75.00 mg, 0.150 mmol) in DCM (5 mL) was added TFA (1 mL) dropwise at rt under nitrogen atmosphere. The resulting mixture was stirred for 1 h at rt. On completion, the reaction mixture was concentrated under reduced pressure. The residue was purified by trituration with Et₂O (10 mL) to afford the title compound (74 mg) as a white solid. LC/MS (ESI, m/z): [(M+H)]⁺=400.2.

### Example 1.157. Preparation of 3-(1-(2,6-dioxopiperidin-3-yl)-3-methyl-2-oxo-2,3-dihydro-1H-benzo[d]imidazol-5-yl)propiolaldehyde (Intermediate FA)

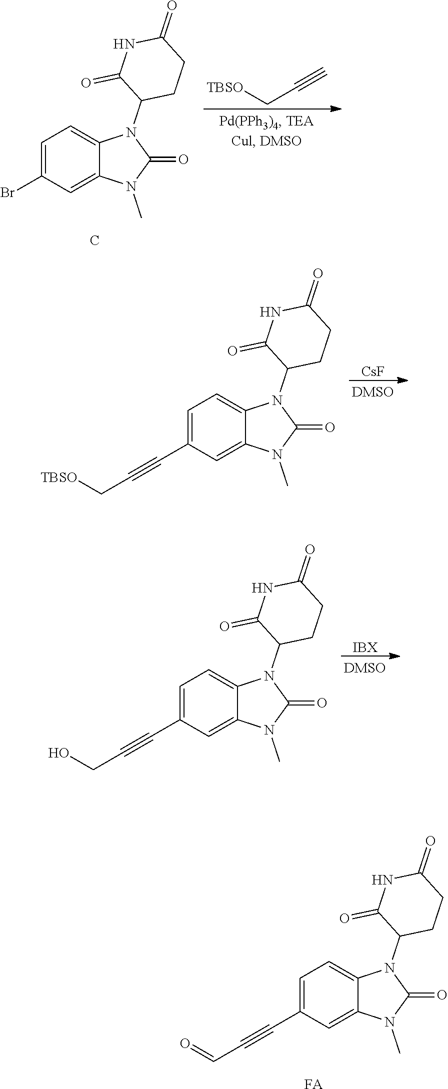

### Step 1—3-(5-(3-((tert-butyldimethylsilyl)oxy)prop-1-yn-1-yl)-3-methyl-2-oxo-2,3-dihydro-1H-benzo[d]imidazol-1-yl)piperidine-2,6-dione

To a solution of 3-(5-bromo-3-methyl-2-oxo-2,3-dihydro-1H-benzo[d]imidazol-1-yl)piperidine-2,6-dione (20.0 g, 59.1 mmol, Intermediate C) and tert-butyldimethyl-prop-2-ynoxy-silane (30.2 g, 177 mmol) in DMSO (300 mL) was added TEA (29.9 g, 296 mmol), Pd(PPh₃)₄ (6.83 g, 5.91 mmol) and CuI (2.25 g, 11.8 mmol) under N₂. The mixture was stirred at 85° C. for 12 h. On completion, the reaction mixture was diluted with sat. NH₄Cl (200 mL), and extracted with EtOAc (250 mL×3). The combined organic layers were washed with brine (150 mL×3), dried over Na₂SO₄ and evaporated. The crude product was purified by column chromatography (SiO₂, Petroleum Ether/Ethyl acetate=1/1 to 2/1) to afford title compound (18.0 g, 71% yield) as a yellow solid. ¹H NMR (400 MHz, DMSO-d₆) δ=11.11 (br s, 1H), 7.31-7.31 (m, 1H), 7.28 (s, 1H), 7.13 (s, 2H), 5.38 (br dd, J=4.4, 12.4 Hz, 1H), 4.55 (s, 2H), 3.34 (br s, 3H), 2.96-2.83 (m, 1H), 2.74-2.60 (m, 2H), 2.03 (br d, J=6.8 Hz, 1H), 0.90 (s, 9H), 0.14 (s, 6H).

### Step 2—3-(5-(3-Hydroxyprop-1-yn-1-yl)-3-methyl-2-oxo-2,3-dihydro-1H-benzo[d]imidazol-1-yl)piperidine-2,6-dione

To a solution of 3-(5-(3-((tert-butyldimethylsilyl)oxy)prop-1-yn-1-yl)-3-methyl-2-oxo-2,3-dihydro-1H-benzo[d]imidazol-1-yl)piperidine-2,6-dione (18.0 g, 42.1 mmol) in DMSO (180 mL) was added CsF (32.0 g, 210 mmol). The mixture was stirred at 25° C. for 2 h. On completion, the reaction mixture was diluted with sat. NH₄Cl (300 mL), then extracted with EtOAc (300 mL×15). The combined organic layers were dried over Na₂SO₄, evaporated and freeze-dried to afford title compound (13.0 g, 49% yield) as a yellow solid. LC-MS (ESI⁺) m/z 314.0 (M+H)⁺.

### Step 3—3-(1-(2,6-Dioxopiperidin-3-yl)-3-methyl-2-oxo-2,3-dihydro-1H-benzo[d]imidazol-5-yl)propiolaldehyde

To a solution of 3-(5-(3-hydroxyprop-1-yn-1-yl)-3-methyl-2-oxo-2,3-dihydro-1H-benzo[d]imidazol-1-yl)piperidine-2,6-dione (13.0 g, 20.8 mmol) in DMSO (130 mL) was added IBX (11.6 g, 41.5 mmol). The mixture was stirred at 25° C. for 2 h. On completion, the reaction mixture was diluted with sat. NH₄Cl (150 mL), then extracted with EtOAc (200 mL×4). The combined organic layers were washed with brine (150 mL×5), dried over Na₂SO₄ and evaporated to afford the title compound (17.0 g, 79% yield) as a yellow solid. ¹H NMR (400 MHz, DMSO-d₆) δ=11.21 (s, 1H), 9.49-9.47 (m, 1H), 8.06-8.04 (m, 1H), 7.52-7.50 (m, 1H), 7.34 (d, J=8.0 Hz, 1H), 5.51 (dd, J=5.2, 12.8 Hz, 1H), 2.97-2.91 (m, 1H), 2.84-2.73 (m, 2H), 2.12-2.08 (m, 1H).

### Example 1.158. Preparation of 3-(3-methyl-2-oxo-5-(3-(piperazin-1-yl)prop-1-yn-1-yl)-2,3-dihydro-1H-benzo[d]imidazol-1-yl)piperidine-2,6-dione (Intermediate FB)

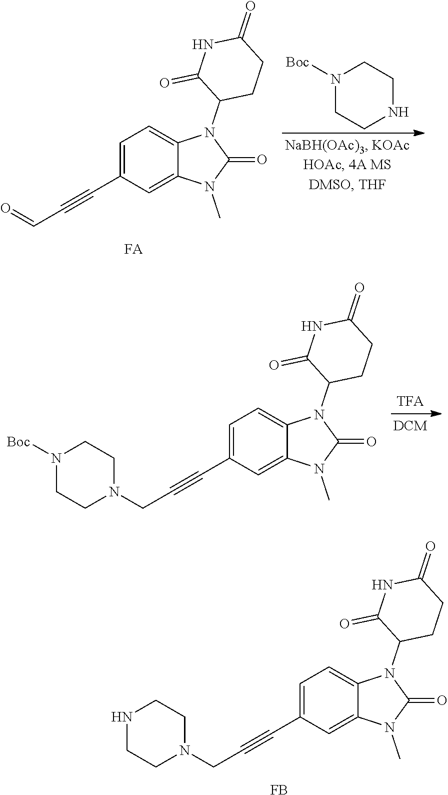

### Step 1—Tert-butyl 4-(3-(1-(2,6-dioxopiperidin-3-yl)-3-methyl-2-oxo-2,3-dihydro-1H-benzo[d]imidazol-5-yl)prop-2-yn-1-yl)piperazine-1-carboxylate

To a solution of 3-(1-(2,6-dioxopiperidin-3-yl)-3-methyl-2-oxo-2,3-dihydro-1H-benzo[d]imidazol-5-yl)propiolaldehyde (17.0 g, 16.4 mmol, Intermediate FA) in DMSO (100 mL) and THF (100 mL) was added KOAc (4.82 g, 49.2 mmol), 4 Å molecular sieves (20.0 g), AcOH (2.95 g, 49.2 mmol), and tert-butyl piperazine-1-carboxylate (7.30 g, 32.8 mmol, HCl salt) at 0° C. and the mixture was stirred at 0° C. for 0.5 h. Then NaBH(OAc)₃ (10.4 g, 49.2 mmol) was added to at 0° C. and the reaction mixture was stirred at 25° C. for 1 h. On completion, the mixture was filtered and diluted with sat. NH₄Cl (100 mL), then extracted with EtOAc (150 mL×3). The combined organic layers were washed with brine (80 mL×4), dried over Na₂SO₄ and evaporated. The residue was purified by column chromatography (SiO₂, Petroleum ether/Ethyl acetate=0/1) to give the title compound (2.80 g, 36% yield) as a yellow solid. ¹H NMR (400 MHz, DMSO-d₆) δ=11.11 (s, 1H), 7.29 (s, 1H), 7.12 (d, J=1.6 Hz, 2H), 5.38 (br dd, J=5.2, 12.8 Hz, 1H), 3.54 (s, 2H), 3.38-3.35 (m, 5H), 2.93-2.84 (m, 1H), 2.75-2.56 (m, 3H), 2.48 (br s, 3H), 2.03 (br d, J=6.0 Hz, 1H), 1.39 (s, 9H).

### Step 2—3-(3-Methyl-2-oxo-5-(3-(piperazin-1-yl)prop-1-yn-1-yl)-2,3-dihydro-1H-benzo[d]imidazol-1-yl)piperidine-2,6-dione

To a solution of tert-butyl 4-(3-(1-(2,6-dioxopiperidin-3-yl)-3-methyl-2-oxo-2,3-dihydro-1H-benzo[d]imidazol-5-yl)prop-2-yn-1-yl)piperazine-1-carboxylate (2.80 g, 5.81 mmol) in DCM (30 mL) was added TFA (9.24 g, 81.0 mmol) and the reaction mixture was stirred at 25° C. for 2 h. On completion, the mixture was filtered and concentrated to give the title compound (3.10 g, TFA salt) as a yellow solid. LC-MS (ESI⁺) m/z 381.8 (M+H)⁺; ¹H NMR (400 MHz, DMSO-d₆) δ=11.12 (s, 1H), 8.87 (br s, 2H), 7.31 (s, 1H), 7.20-7.14 (m, 2H), 5.40 (dd, J=5.2, 12.8 Hz, 1H), 3.89 (s, 2H), 3.35 (s, 3H), 3.25 (br s, 4H), 3.01 (br s, 4H), 2.93-2.85 (m, 1H), 2.77-2.58 (m, 3H), 2.06-2.00 (m, 1H).

### Example 1.159. Preparation of 2-[1-(2,6-dioxo-3-piperidyl)-3-methyl-2-oxo-benzimidazol-5-yl]acetaldehyde (Intermediate FC)

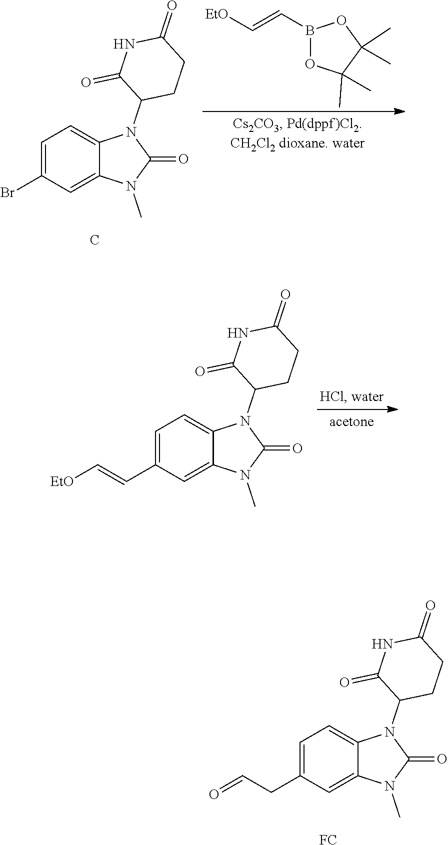

### Step 1—(E)-3-(5-(2-ethoxyvinyl)-3-methyl-2-oxo-2,3-dihydro-1H-benzo[d]imidazol-1-yl)piperidine-2,6-dione

A mixture of 3-(5-bromo-3-methyl-2-oxo-benzimidazol-1-yl)piperidine-2,6-dione (7 g, 20.7 mmol, Intermediate C), 2-[(E)-2-ethoxyvinyl]-4,4,5,5-tetramethyl-1,3,2-dioxaborolane (8.20 g, 41.4 mmol), cesium carbonate (13.4 g, 41.4 mmol), [1,1-bis(diphenylphino)ferrocene]pallidum, (II)chloride (1.69 g, 2.07 mmol) in dioxane (70 mL) and water (10 mL) was degassed and purged with nitrogen three times. Then the mixture was stirred at 80° C. for 2 hours under nitrogen atmosphere. The reaction mixture was diluted with water (10 mL), and extracted with ethyl acetate (30 mL×3). The organic layer was dried over anhydrous sodium sulfate, and concentrated under reduced pressure to give a residue. The crude product was purified by reversed-phase HPLC (0.1% FA condition) to give the title compound (3 g, 43% yield) as a yellow solid. LCMS=330.4[M+H]⁺.

### Step 2—2-[1-(2,6-dioxo-3-piperidyl)-3-methyl-2-oxo-benzimidazol-5-yl]acetaldehyde

To a solution of 3-[5-[(E)-2-ethoxyvinyl]-3-methyl-2-oxo-benzimidazol-1-yl]piperidine-2,6-dione (2.8 g, 8.50 mmol) in acetone (60 mL) was added water (60 mL) and hydrochloric acid (60 mL) at 0° C. The mixture was stirred at 2 5° C. for 1 hour under nitrogen. The reaction mixture was diluted with water (10 mL), and extracted with dichloromethane (100 mL×3). The organic layer was dried over anhydrous sodium sulfate, and concentrated under reduced pressure to give the title compound (2 g) as a white solid. LCMS=302.5[M+H]⁺.

### Example 1.160. Preparation of 3-{3-methyl-2-oxo-5-[2-(piperazin-1-yl)ethyl]-1,3-benzodiazol-1-yl}piperidine-2,6-dione (Intermediate FD)

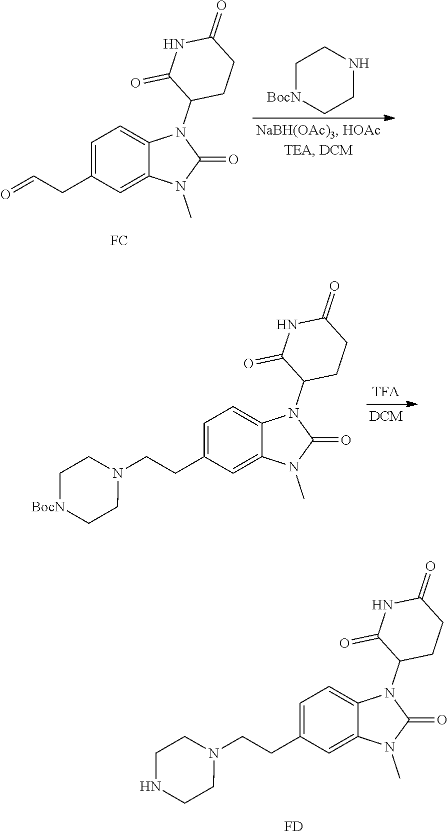 [SMILES: Cn1c(=O)n(C2CCC(=O)NC2=O)c2ccc(CC=O)cc21 ; CC(C)(C)OC(=O)N1CCNCC1 ; "FC" ; Cn1c(=O)n(C2CCC(=O)NC2=O)c2ccc(CCN3CCN(C(=O)OC(C)(C)C)CC3)cc21 ; Cn1c(=O)n(C2CCC(=O)NC2=O)c2ccc(CCN3CCNCC3)cc21 ; "FD"]

### Step 1—Tert-butyl 4-(2-(1-(2,6-dioxopiperidin-3-yl)-3-methyl-2-oxo-2,3-dihydro-1H-benzo[d]imidazol-5-yl)ethyl)piperazine-1-carboxylate

To a solution of tert-butyl piperazine-1-carboxylate (1.63 g, 7.30 mmol), 2-[1-(2,6-dioxo-3-piperidyl)-3-methyl-2-oxo-benzimidazol-5-yl]acetaldehyde (2.00 g, 6.64 mmol, Intermediate FC), triethylamine (671 mg, 6.64 mmol) in dichloromethane (40 mL) was stirred for 1 hour at 25° C. Then acetic acid (398 mg, 6.64 mmol) and sodium triacethoxy borohydride (2.81 g, 13.2 mmol) were added and the mixture was stirred at 25° C. for 11 hour. On completion, the reaction mixture was diluted with water (10 mL), and extracted with ethyl acetate (30 mL×3), dried over anhydrous sodium sulfate, and concentrated under reduced pressure to give a residue. The residue was purified by perp-HPLC (column: Phenomenex luna C18 150*40 mm*15 um; mobile phase: [water (FA)-ACN]; B %: 5%-35%, 10 min) to give the title compound (1.25 g, 39% yield) as a white solid. ¹H NMR (400 MHz, DMSO-d₆) δ=11.07 (s, 1H), 7.06 (s, 1H), 7.00 (d, J=8.0 Hz, 1H), 6.89 (d, J=7.6 Hz, 1H), 5.34-5.30 (m, 1H), 3.32 (s, 6H), 2.98-2.52 (m, 8H), 2.40 (s, 4H), 2.05-1.94 (m, 1H), 1.39 (s, 9H); LCMS=472.0 [M+H]⁺.

### Step 2—3-{3-methyl-2-oxo-5-[2-(piperazin-1-yl)ethyl]-1,3-benzodiazol-1-yl}piperidine-2,6-dione Trifluoroacetate

To a stirred mixture of tert-butyl 4-{2-[1-(2,6-dioxopiperidin-3-yl)-3-methyl-2-oxo-1,3-benzodiazol-5-yl]ethyl}piperazine-1-carboxylate (75 mg, 0.16 mmol) in DCM (2 mL) was added TFA (0.4 mL, 5 mmol) dropwise at rt under nitrogen atmosphere. The resulting mixture was stirred for 1 h at rt under nitrogen atmosphere. On completion, the reaction mixture was concentrated under reduced pressure. The residue was purified by trituration with Et₂O to give the title compound (74 mg) as a yellow solid. LC/MS (ESI, m/z): [(M+H)]⁺=372.2.

### Example 1.161. Preparation of tert-butyl 2-(prop-2-yn-1-yl)-5-oxa-2,8-diazaspiro[3.5]nonane-8-carboxylate (Intermediate FE)

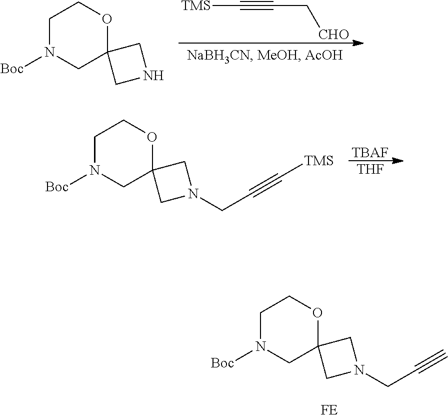

### Step 1—Tert-butyl 2-(3-(trimethylsilyl)prop-2-yn-1-yl)-5-oxa-2,8-diazaspiro[3.5]nonane-8-carboxylate

A solution of tert-butyl 5-oxa-2, 8-diazaspiro[3.5]nonane-8-carboxylate (3.30 g, 14.5 mmol, CAS #1251005-61-4), 3-trimethylsilylprop-2-ynal (1.82 g, 14.5 mmol, CAS #2975-46-4) and AcOH (86.8 mg, 1.45 mmol, 82.7 μL) in MeOH (30 mL) was stirred at 25° C. for 0.5 hr. Then sodium cyanoboranuide (1.82 g, 28.9 mmol) was added to the mixture at 0° C. and the resulting mixture was stirred at 25° C. for 12 hr. On completion, the reaction mixture was poured into H₂O (40 mL) and separated, the water phase was extracted with EA (50 mL×2). The combined organic layers were dried over Na₂SO₄, filtered and concentrated under reduced pressure to give a residue. The residue was purified by column chromatography (SiO₂, PE:EA=3:1 to 1:1, Rf=0.5 (PE:EA=3:1)) to give the title compound (3.2 g, 65% yield) was obtained as yellow oil. ¹H NMR (400 MHz, CDCl₃-d) δ=3.65-3.52 (m, 4H), 3.46-3.30 (m, 6H), 3.18 (br d, J=8.0 Hz, 2H), 1.48 (s, 9H), 0.19 (s, 9H).

### Step 2—tert-butyl 2-(prop-2-yn-1-yl)-5-oxa-2,8-diazaspiro[3.5]nonane-8-carboxylate

To a solution of tert-butyl 2-(3-trimethylsilylprop-2-ynyl)-5-oxa-2, 8-diazaspiro[3.5]nonane-8-carboxylate (3.20 g, 9.45 mmol) in THF (40 mL) was added TBAF (1 M, 47.3 mL). The mixture was stirred at 25° C. for 2 hr. On completion, the reaction mixture was poured into H₂O (50 mL) and separated, and the water phase was extracted with EA (60 mL×3). The combined organic layers were dried over Na₂SO₄, filtered and concentrated under reduced pressure to give a residue. The residue was purified by column chromatography (SiO₂, PE:EA=3:1 to 1:1, P:Rf=0.2 (PE:EA=3:1) to give the title compound (2.3 g, 91% yield) as yellow solid. ¹H NMR (400 MHz, CDCl₃-d) δ=3.48-3.38 (m, 4H), 3.30-3.15 (m, 6H), 3.04 (d, J=8.4 Hz, 2H), 2.14 (t, J=2.4 Hz, 1H), 1.32 (s, 9H).

### Example 1.162. Preparation of 3-(5-{3-[6-(4-aminocyclohexanecarbonyl)-2,6-diazaspiro[3.4]octan-2-yl]prop-1-yn-1-yl}-3-methyl-2-oxo-1,3-benzodiazol-1-yl)piperidine-2,6-dione (Intermediate FF)

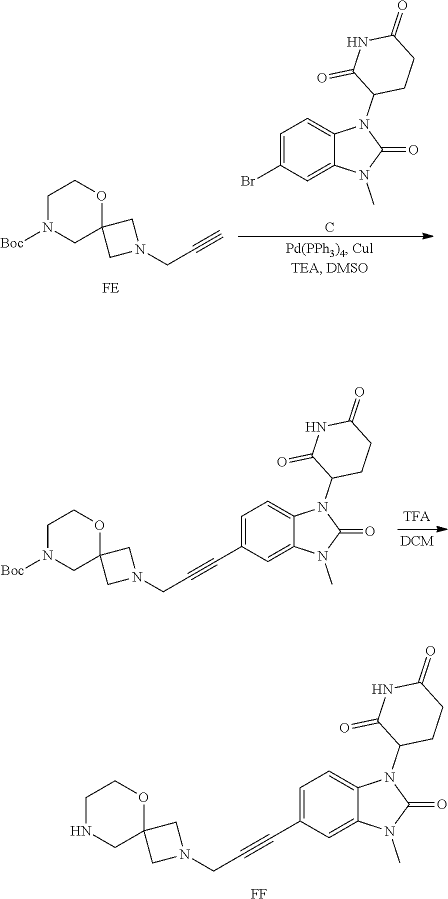

### Step 1—Tert-butyl 2-(3-(1-(2,6-dioxopiperidin-3-yl)-3-methyl-2-oxo-2,3-dihydro-1H-benzo[d]imidazol-5-yl)prop-2-yn-1-yl)-5-oxa-2,8-diazaspiro[3.5]nonane-8-carboxylate

To a solution of tert-butyl 2-prop-2-ynyl-5-oxa-2, 8-diazaspiro[3.5]nonane-8-carboxylate (1.73 g, 6.51 mmol, Intermediate FE), 3-(5-bromo-3-methyl-2-oxo-benzimidazol-1-yl) piperidine-2, 6-dione (2.20 g, 6.51 mmol, Intermediate C), CuI (124 mg, 651 μmol) and Pd(PPh₃)₄ (752 mg, 651 μmol) in DMSO (20 mL) was added TEA (3.29 g, 32.5 mmol, 4.53 mL). The mixture was stirred at 80° C. under N₂ for 2 hr. On completion, the reaction was quenched by addition of saturated NH₄Cl aqueous (60 mL), and extracted with EA (100 mL×3). The organic layer was dried over Na₂SO₄, filtered and the filtration was concentrated to give a residue. The residue was purified by column chromatography (SiO₂, PE:EA=1:1 to 0:1, P: Rf=0.5 (PE:EA=0:1)) to give crude product (5 g, 64% purity). The product was then purified via reversed-phase HPLC (0.1% FA) to give the title compound. ¹H NMR (400 MHz, CDCl₃-d) δ=8.20 (br s, 1H), 7.26-7.06 (m, 2H), 6.76 (d, J=8.4 Hz, 1H), 5.21 (dd, J=5.6, 12.8 Hz, 1H), 3.69 (s, 2H), 3.64-3.58 (m, 6H), 3.45 (s, 5H), 3.40 (br d, J=4 Hz, 2H), 3.01-2.93 (m, 1H), 2.89-2.67 (m, 2H), 2.31-2.21 (m, 1H), 1.48 (s, 9H).

### Step 2—3-(5-{3-[6-(4-Aminocyclohexanecarbonyl)-2,6-diazaspiro[3.4]octan-2-yl]prop-1-yn-1-yl}-3-methyl-2-oxo-1,3-benzodiazol-1-yl)piperidine-2,6-dione Trifluoroacetate

To a stirred solution of tert-butyl N-[4-(2-{3-[1-(2,6-dioxopiperidin-3-yl)-3-methyl-2-oxo-1,3-benzodiazol-5-yl]prop-2-yn-1-yl}-2,6-diazaspiro[3.4]octane-6-carbonyl)cyclohexyl]carbamate (50 mg, 0.079 mmol) in DCM (1.5 mL) was added TFA (0.5 mL) dropwise at rt under nitrogen atmosphere. The resulting mixture was stirred for 1 h at rt under nitrogen atmosphere. On completion, the reaction mixture was concentrated under reduced pressure. The residue was purified by trituration with ethyl ether (10 mL). The precipitated solids were collected by filtration and washed with ethyl ether (3×5 mL) to afford the title compound (50 mg) as a white solid. LC/MS (ESI, m/z): [(M+1)]⁺=533.3.

### Example 1.163. Preparation of 3-[3-Methyl-2-oxo-5-(2-trimethylsilylethynyl)benzimidazol-1-yl]piperidine-2,6-dione (Intermediate FG)

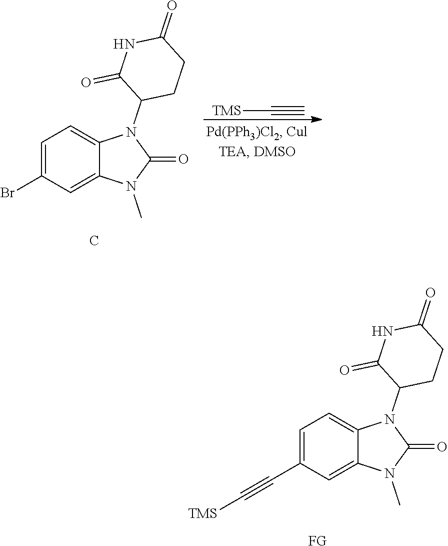

To a solution of 3-(5-bromo-3-methyl-2-oxo-benzimidazol-1-yl) piperidine-2, 6-dione (2.00 g, 5.91 mmol, Intermediate C), ethynyl (trimethyl) silane (1.74 g, 17.7 mmol, 2.46 mL) Pd(PPh₃)₄ (683 mg, 591 μmol) in DMSO (12 mL) was added CuI (113 mg, 591.44 μmol) and Et₃N (1.80 g, 17.7 mmol, 2.47 mL) at 25° C. The mixture was then heated to 100° C. for 2 hr. On completion, the reaction mixture was concentrated under reduced pressure. The residue was diluted with H₂O (200 mL) and extracted with EA (200 mL×3). The combined organic layers were washed with H₂O (300 mL×2), dried over Na₂SO₄, filtered and concentrated under reduced pressure to give a residue. The reaction mixture was diluted with (EA:PE=2/1=300 mL), filtered and the cake was dried under reduced pressure to give the title compound (5.5 g, 79% yield) as a yellow solid. ¹H NMR (400 MHz, DMSO-d₆) δ=11.12 (s, 1H), 7.33 (s, 1H), 7.21-7.10 (m, 2H), 5.75 (s, 1H), 3.46-3.38 (s, 3H), 2.95-2.82 (m, 2H), 2.76-2.59 (m, 2H), 2.07-1.99 (m, 1H), 0.36-0.15 (m, 9H); LC-MS (ESI⁺) m/z 356.1 (M+H)⁺.

### Example 1.164. Preparation of 3-[5-[2-(2-Chloropyrimidin-5-yl)ethynyl]-3-methyl-2-oxo-benzimidazol-1-yl]piperidine-2, 6-dione (Intermediate FH)

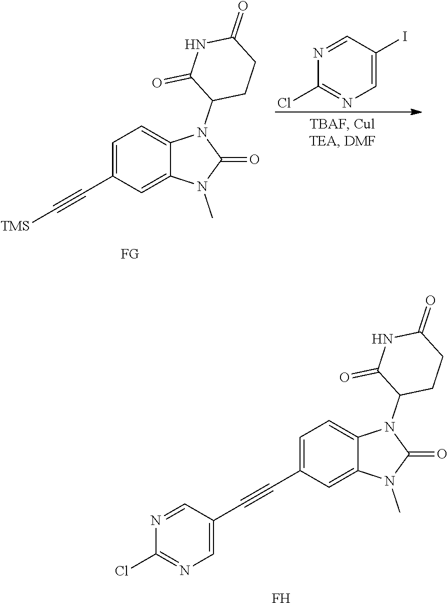

To a solution of 3-[3-methyl-2-oxo-5-(2-trimethylsilylethynyl) benzimidazol-1-yl]piperidine-2, 6-dione (5.00 g, 14.0 mmol, Intermediate FG), 2-chloro-5-iodo-pyrimidine (4.73 g, 19.6 mmol) CuI (401 mg, 2.11 mmol) in DMF (80 mL) was added TBAF (1 M, 14.0 mL) and Et₃N (2.85 g, 28.1 mmol, 3.92 mL) at 25° C. The mixture was stirred at 60° C. for 12 hr. On completion, the reaction mixture was quenched by addition of EA (500 mL), and then diluted with H₂O (500 mL) and extracted with EA (200 mL×3). The combined organic layers were dried over Na₂SO₄, filtered and concentrated under reduced pressure to give a residue. Next, DCM (100 mL) was added and then filtered and the cake was dried under reduced pressure to give the title compound (2.7 g, 43% yield) as a white solid. LC-MS (ESI⁺) m/z 396.8 (M+H)⁺.

### Example 1.165. Preparation of 3-{5-[2-(2-{1,6-diazaspiro[3.3]heptan-6-yl}pyrimidin-5-yl)ethynyl]-3-methyl-2-oxo-1,3-benzodiazol-1-yl}piperidine-2,6-dione (Intermediate FI)

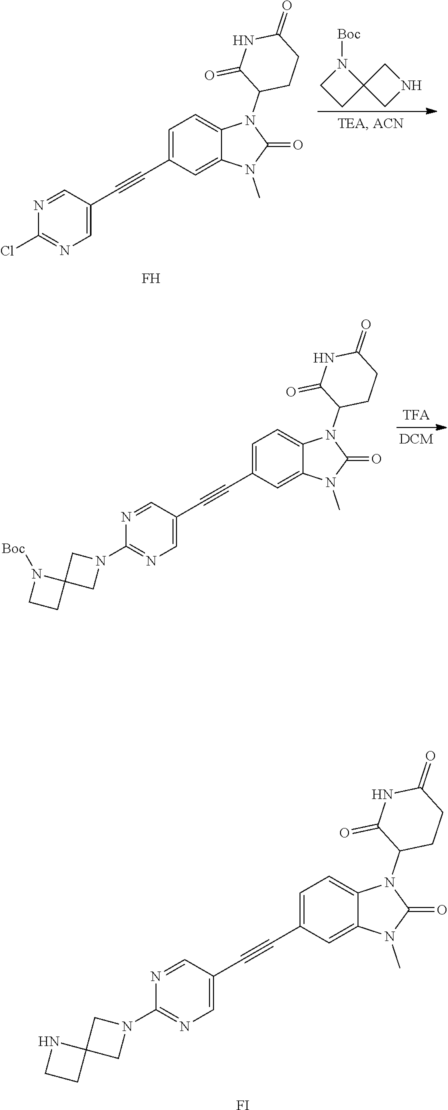

### Step 1—Tert-butyl 6-(5-((1-(2,6-dioxopiperidin-3-yl)-3-methyl-2-oxo-2,3-dihydro-1H-benzo[d]imidazol-5-yl)ethynyl)pyrimidin-2-yl)-1,6-diazaspiro[3.3]heptane-1-carboxylate

To a solution of 3-[5-[2-(2-chloropyrimidin-5-yl) ethynyl]-3-methyl-2-oxo-benzimidazol-1-yl]piperidine-2, 6-dione (580 mg, 1.47 mmol, Intermediate FG) and tert-butyl 1, 6-diazaspiro[3.3]heptane-1-carboxylate (422.47 mg, 1.47 mmol, oxalic acid, CAS #1523571-10-9) in ACN (10 mL) was added TEA (445 mg, 4.40 mmol, 612 μL). The mixture was stirred at 50° C. under N₂ for 12 hr. On completion, the mixture was filtered. The crude product was triturated with EA (10 mL) at 25° C. for 3 min to give the title compound (1.02 g, quant. yield) as white solid. ¹H NMR (400 MHz, DMSO-d₆) δ=11.13 (s, 1H), 8.54 (s, 2H), 7.39 (s, 1H), 7.30-7.09 (m, 2H), 5.40 (dd, J=5.2, 12.8 Hz, 1H), 4.47 (br d, J=10.0 Hz, 2H), 4.22 (br d, J=10 Hz, 2H), 3.69 (br t, J=6.8 Hz, 2H), 3.36 (s, 3H), 2.99-2.82 (m, 1H), 2.78-2.61 (m, 2H), 2.48-2.44 (m, 2H), 2.11-1.98 (m, 1H), 1.39 (br s, 2H), 1.32-1.05 (m, 7H).

### Step 2—3-{5-[2-(2-{1,6-Diazaspiro[3.3]heptan-6-yl}pyrimidin-5-yl)ethynyl]-3-methyl-2-oxo-1,3-benzodiazol-1-yl}piperidine-2,6-dione trifluoroacetate

To a stirred solution of tert-butyl 6-(5-{2-[1-(2,6-dioxopiperidin-3-yl)-3-methyl-2-oxo-1,3-benzodiazol-5-yl]ethynyl}pyrimidin-2-yl)-1,6-diazaspiro[3,3]heptane-1-carboxylate (50 mg, 0.09 mmol) in DCM (1.5 mL) was added TFA (0.5 mL) dropwise at rt under nitrogen atmosphere. The resulting mixture was stirred for 1 h at rt under nitrogen atmosphere. On completion, the reaction mixture was concentrated under reduced pressure. The residue was purified by trituration with ethyl ether (10 mL). The precipitated solids were collected by filtration and washed with ethyl ether (3×5 mL) to afford the title compound (50 mg) as a white solid. LC/MS (ESI, m/z): [(M+1)]⁺=458.2.

### Example 1.166. Preparation of 4-((3-fluoro-2-methoxyphenyl)amino)-6-((5-formylpyridin-2-yl)amino)-N-(methyl-d3)pyridazine-3-carboxamide (Intermediate FJ)

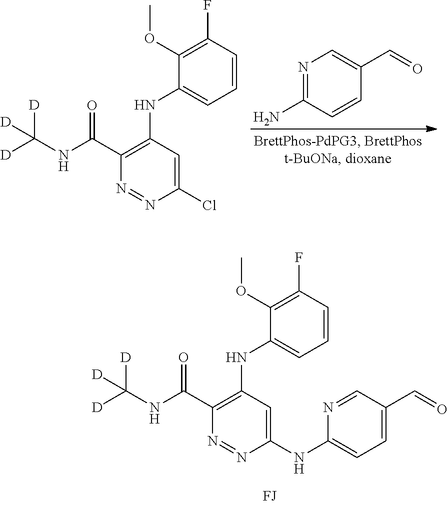 [SMILES: [2H]C([2H])([2H])NC(=O)c1nnc(Cl)cc1Nc1cccc(F)c1OC ; Nc1ccc(C=O)cn1 ; [2H]C([2H])([2H])NC(=O)c1nnc(Nc2ccc(C=O)cn2)cc1Nc1cccc(F)c1OC]

To a stirred mixture of 6-chloro-4-((3-fluoro-2-methoxyphenyl)amino)-N-(methyl-d3)pyridazine-3-carboxamide (200.00 mg, 0.637 mmol, synthesized via Steps 1-2 of Intermediate AF) and 6-aminopyridine-3-carbaldehyde (194.64 mg, 1.593 mmol) in 1,4-dioxane (5 mL) were added BrettPhos Pd G3 (57.79 mg, 0.064 mmol), BrettPhos (34.22 mg, 0.064 mmol) and t-BuONa (122.53 mg, 1.274 mmol) in turns at rt. The resulting mixture was stirred for 2 h at 100° C. under nitrogen atmosphere. On completion, the mixture was cooled to rt and concentrated under reduced pressure. The residue was purified by reverse phase flash chromatography (Column: WelFlash™ C18-I, 20-40 um, 120 g; Eluent A: Water (plus 10 mmol/L FA); Eluent B: ACN; Gradient: 25%-60% B in 25 min; Flow rate: 60 mL/min; Detector: 220/254 nm; desired fractions were collected at 58% B) and concentrated under reduced pressure to afford the title compound (120.00 mg, 47% yield) as a light yellow solid. LC/MS (ESI, m/z): [(M+H)]⁺=400.1.

### Example 1.167. Preparation of 3-{3-methyl-2-oxo-4-[1-(piperidin-4-yl)azetidin-3-yl]-1,3-benzodiazol-1-yl}piperidine-2,6-dione (Intermediate FK)

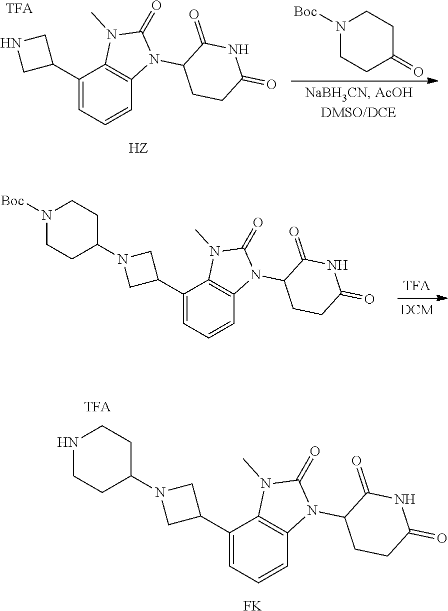

### Step 1—Tert-butyl 4-{3-[1-(2,6-dioxopiperidin-3-yl)-3-methyl-2-oxo-1,3-benzodiazol-4-yl]azetidin-1-yl}piperidine-1-carboxylate

To a stirred mixture of 3-[4-(azetidin-3-yl)-3-methyl-2-oxo-1,3-benzodiazol-1-yl]piperidine-2,6-dione trifluoroacetate (600.00 mg, 1.455 mmol, Intermediate HZ) and tert-butyl 4-oxopiperidine-1-carboxylate (347.89 mg, 1.746 mmol) in DMSO (6 mL) and DCE (6 mL) was added AcOH (0.25 mL, 4.365 mmol). The resulting mixture was stirred for 30 min at rt under nitrogen atmosphere. To the above mixture was added NaBH₃CN (457.16 mg, 7.275 mmol) at 0° C. The resulting mixture was stirred for additional 16 h at rt. On completion, the reaction mixture was concentrated under reduced pressure. The residue was purified by reverse phase flash chromatography (Column: WelFlash™ C18-I, 20-40 um, 330 g; Eluent A: Water (plus 10 mmol/L FA); Eluent B: ACN; Gradient: 25%-55% B in 25 min; Flow rate: 80 mL/min; Detector: 220/254 nm; desired fractions were collected at 45% B) and concentrated under reduced pressure to afford the title compound (200 mg, 28% yield) as a yellow oil. LC/MS (ESI, m/z): [(M+H)]⁺=498.2.

### Step 2—3-{3-Methyl-2-oxo-4-[1-(piperidin-4-yl)azetidin-3-yl]-1,3-benzodiazol-1-yl}piperidine-2,6-dione Trifluoroacetate

To a stirred mixture of tert-butyl 4-{3-[1-(2,6-dioxopiperidin-3-yl)-3-methyl-2-oxo-1,3-benzodiazol-4-yl]azetidin-1-yl}piperidine-1-carboxylate (200.00 mg, 0.402 mmol) in DCM (6 mL) was added TFA (2 mL) dropwise at rt. The resulting mixture was stirred for 1 h at rt. On completion, the reaction mixture was concentrated under reduced pressure. The residue was purified by trituration with Et₂O (15 mL) to afford the title compound (190.00 mg) as a brown yellow oil. LC/MS (ESI, m/z): [(M+H)]⁺=398.2.

### Example 1.168. Preparation of 3-[3-methyl-2-oxo-4-(3-piperidyl)benzimidazol-1-yl]piperidine-2,6-dione (Intermediate FL)

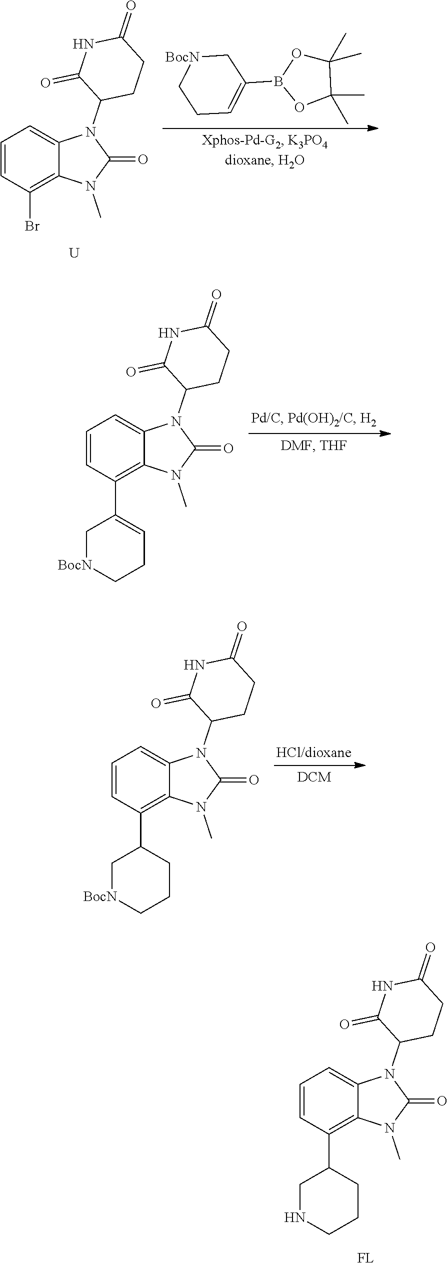

### Step 1—Tert-butyl 5-[1-(2,6-dioxo-3-piperidyl)-3-methyl-2-oxo-benzimidazol-4-yl]-3,6-dihydro-2H-pyridine-1-carboxylate

A mixture of 3-(4-bromo-3-methyl-2-oxo-benzimidazol-1-yl)piperidine-2,6-dione (7.5 g, 22 mmol, Intermediate U), tert-butyl-5-(4,4,5,5-tetramethyl-1,3,2-dioxaborolan-2-yl)-3,6-dihydro-2H-pyridine-1-carboxylate (10.29 g, 33.2 mmol, CAS #885693-20-9), chloro (2-dicyclohexylphosphino-2′,4′,6′-triisopropyl-1,1′-biphenyl) [2-(2′-amino-1,1′-biphenyl)]palladium (II) (1.75 g, 2.22 mmol), and potassium phosphate (9.42 g, 44.3 mmol) in dioxane (400 mL) and water (40 mL) was degassed and purged with nitrogen three times. Then the mixture was stirred at 80° C. for 4 hours under nitrogen atmosphere. The reaction mixture was diluted with water (100 mL) and extracted with ethyl acetate (200 mL×3). The combined organic layers were washed with brine (100 mL×3), dried over anhydrous sodium sulfate, filtered and concentrated under reduced pressure to give a residue. The crude product was triturated with ethyl acetate (50 mL) at 25° C. for 30 min to give the title compound (4.9 g, 50% yield) as a white solid. LC-MS (ESI+) m/z 385.2 (M−55)⁺.

### Step 2—Tert-butyl 3-[1-(2,6-dioxo-3-piperidyl)-3-methyl-2-oxo-benzimidazol-4-yl]piperidine-1-carboxylate

Nitrogen was first passed into the reaction vessel, and then Pd/C (2.5 g, 227 μmol, 10 wt %) and dihydroxypalladium (2.5 g, 20 wt %) was added to it. Then tetrahydrofuran (150 mL) was added by drip washing the vessel wall. Next, a solution of tert-butyl 5-[1-(2,6-dioxo-3-piperidyl)-3-methyl-2-oxo-benzimidazol-4-yl]-3,6-dihydro-2H-pyridine-1-carboxylate (4.9 g, 11.1 mmol) in dimethylformamide (300 mL) and tetrahydrofuran (150 mL) was added to the reaction vessel under nitrogen. Then the mixture was degassed and purged with hydrogen three times at 25° C. Next, the mixture was stirred at 70° C. for 12 hours under hydrogen at 50 psi. On completion, the reaction mixture was filtered, and the filtered cake was washed with tetrahydrofuran (600 mL). The filtered cake was discarded and the filtrate was concentrated under reduced pressure to give the title compound (4 g) as a white solid. LC-MS (ESI+) m/z 387.2 (M−55)⁺.

### Step 3—3-[3-Methyl-2-oxo-4-(3-piperidyl)benzimidazol-1-yl]piperidine-2,6-dione

To a solution of tert-butyl 3-[1-(2,6-dioxo-3-piperidyl)-3-methyl-2-oxo-benzimidazol-4-yl]piperidine-1-carboxylate (7 g, 16 mmol) in dichloromethane (70 mL) was added hydrochloric acid/dioxane (70 mL). The mixture was stirred at 25° C. for 2 hr. On completion, the reaction mixture was concentrated under reduced pressure to give the title compound (4.8 g, HCl) as a white solid. LC-MS (ESI+) m/z 343.2 (M+H)⁺.

### Example 1.169. Preparation of 3-{3-methyl-2-oxo-4-[1-(piperidin-4-ylmethyl)piperidin-3-yl]-1,3-benzodiazol-1-yl}piperidine-2,6-dione (Intermediate FM)

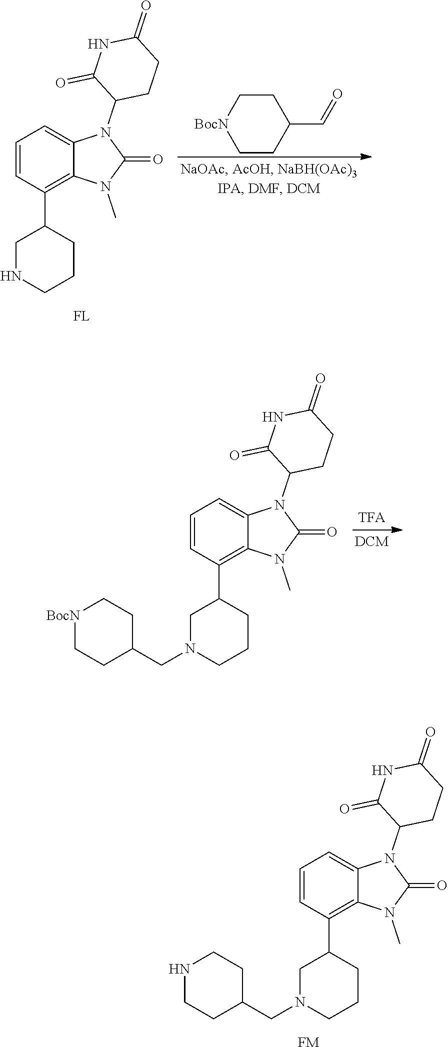

### Step 1—Tert-butyl 4-[[3-[1-(2,6-dioxo-3-piperidyl)-3-methyl-2-oxo-benzimidazol-4-yl]-1-piperidyl]methyl]piperidine-1-carboxylate

To a solution of 3-[3-methyl-2-oxo-4-(3-piperidyl)benzimidazol-1-yl]piperidine-2,6-dione (4.8 g, 13 mmol, Intermediate FL) in dichloromethane (100 mL), isopropyl alcohol (200 mL) and dimethylformamide (100 mL) was added sodium acetate (3.12 g, 38.0 mmol) at 25° C. for 30 minutes. Then to the mixture was added tert-butyl 4-formylpiperidine-1-carboxylate (2.70 g, 12.6 mmol, CAS #137076-22-3) and acetic acid (1.52 g, 25.3 mmol) at 25° C. for 30 minutes. Next, sodium triacethoxy borohydride (5.37 g, 25.3 mmol) was added to the mixture at 0° C. Then the mixture was stirred at 25° C. for 11 hours. On completion, the reaction mixture was quenched with 1N sodium bicarbonate solution to pH=7, and then diluted with water (10 mL) and extracted with ethyl acetate (100 mL×3). The combined organic layers were washed with brine (100 mL×3), dried over anhydrous sodium sulfate, filtered and concentrated under reduced pressure to give a residue. The residue was purified by perp-HPLC (column: Phenomenex Luna C18; 150×40 mm×15 um; mobile phase: [water (TFA)-ACN]; B %: 14%-44%, 10 min) to give the title compound (4.55 g, 54% yield) as a white solid. ¹H NMR (400 MHz, DMSO-d₆) δ=11.11 (s, 1H), 7.13-6.99 (m, 3H), 5.47-5.31 (m, 1H), 3.94-3.85 (m, 3H), 3.64-3.55 (m, 5H), 3.27-3.15 (m, 1H), 3.07-2.84 (m, 4H), 2.80-2.58 (m, 4H), 2.14-1.90 (m, 5H), 1.76 (d, J=11.6 Hz, 2H), 1.66 (d, J=12.8 Hz, 1H), 1.38 (s, 9H), 1.17-0.92 (m, 2H); LC-MS (ESI+) m/z 540.5 (M+H)⁺.

### Step 2—3-{3-Methyl-2-oxo-4-[1-(piperidin-4-ylmethyl)piperidin-3-yl]-1,3-benzodiazol-1-yl}piperidine-2,6-dione Trifluoroacetate

To a stirred solution of tert-butyl 4-({3-[1-(2,6-dioxopiperidin-3-yl)-3-methyl-2-oxo-1,3-benzodiazol-4-yl]piperidin-1-yl}methyl)piperidine-1-carboxylate (45 mg, 0.083 mmol) in DCM (1 mL) was added TFA (0.2 mL) dropwise at rt. The resulting mixture was stirred for 1 h at rt. On completion, the reaction mixture was concentrated under reduced pressure. The residue was triturated with Et₂O to afford the title compound (50 mg) as a white solid. LC/MS (ESI, m/z): [(M+1)]⁺=440.3.

### Example 1.170. Preparation of 3-[3-methyl-2-oxo-5-(pyrrolidin-3-yl)-1,3-benzodiazol-1-yl]piperidine-2,6-dione (Intermediate FN)

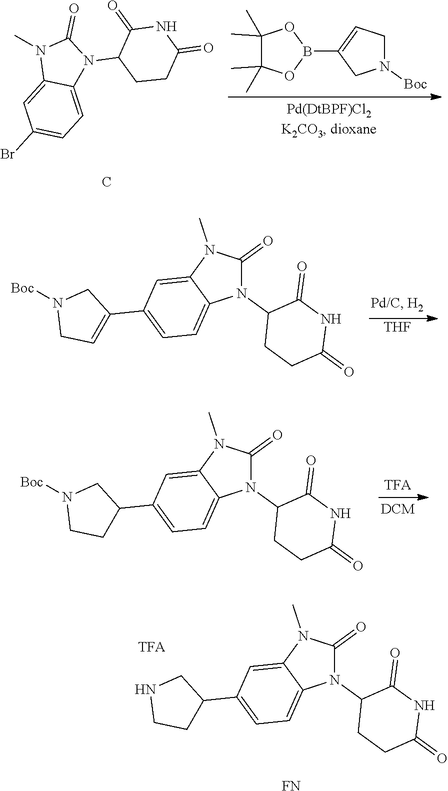

### Step 1—Tert-butyl 3-[1-(2,6-dioxopiperidin-3-yl)-3-methyl-2-oxo-1,3-benzodiazol-5-yl]-2,5-dihydropyrrole-1-carboxylate

To a stirred solution of 3-(5-bromo-3-methyl-2-oxo-1,3-benzodiazol-1-yl)piperidine-2,6-dione (600 mg, 2 mmol, Intermediate C) and tert-butyl 3-(4,4,5,5-tetramethyl-1,3,2-dioxaborolan-2-yl)-2,5-dihydropyrrole-1-carboxylate (628.51 mg, 2.129 mmol) in dioxane (15 mL) and H₂O (3 mL) were added K₂CO₃ (490.44 mg, 3.548 mmol), Pd(dtbpf)Cl₂ (115.64 mg, 0.177 mmol) in turns at rt under nitrogen atmosphere. The resulting mixture was stirred for 1 h at 90° C. under nitrogen atmosphere. On completion, the reaction mixture was cooled to rt and filtered. The resulting mixture was concentrated under reduced pressure. The residue was purified by reverse flash chromatography (column, C18 silica gel; mobile phase A: water (10 mmol/L NH₄HCO₃), mobile phase B: ACN, 30% to 60% gradient in 30 min; detector, UV 254 nm; the fractions were collected at 43%) and concentrated under reduced pressure to afford the title compound (620 mg, 82% yield) as a yellow solid. LC/MS (ESI, m/z): [(M+H)]⁺=427.2.

### Step 2—Tert-butyl 3-[1-(2,6-dioxopiperidin-3-yl)-3-methyl-2-oxo-1,3-benzodiazol-5-yl]pyrrolidine-1-carboxylate

To a stirred solution of tert-butyl 3-[1-(2,6-dioxopiperidin-3-yl)-3-methyl-2-oxo-1,3-benzodiazol-5-yl]-2,5-dihydropyrrole-1-carboxylate (620 mg, 1.45 mmol) in THF (20 mL) was 10 wt % Pd/C (77.36 mg) under nitrogen atmosphere. The reaction system was degassed under vacuum and purged with H₂ several times, then it was hydrogenated under H₂ balloon (1 atm) at 25° C. for 16 h. After completion of the reaction, Pd/C was filtered off through celite and the corresponding filtrate was concentrated under reduced pressure. The residue was purified by trituration with diethyl ether (50 mL) to give the title compound (600 mg, 96% yield) as a light yellow solid. LC/MS (ESI, m/z): [(M+H)]⁺=429.2.

### Step 3—3-[3-Methyl-2-oxo-5-(pyrrolidin-3-yl)-1,3-benzodiazol-1-yl]piperidine-2,6-dione Trifluoroacetate

To a solution of tert-butyl 3-[1-(2,6-dioxopiperidin-3-yl)-3-methyl-2-oxo-1,3-benzodiazol-5-yl]pyrrolidine-1-carboxylate (600 mg, 1 mmol) in DCM (15 mL) was added TFA (3 mL) dropwise at rt. The reaction mixture was stirred for 2 h at rt under nitrogen atmosphere. On completion, the reaction mixture was concentrated under reduced pressure. The residue was triturated with Et₂O to afford the title compound (400 mg) as a purple solid. LC/MS (ESI, m/z): [(M+H)]⁺=329.2.

### Example 1.171. Preparation of 3-[3-methyl-2-oxo-4-(pyrrolidin-3-yl)-1,3-benzodiazol-1-yl]piperidine-2,6-dione (Intermediate FO)

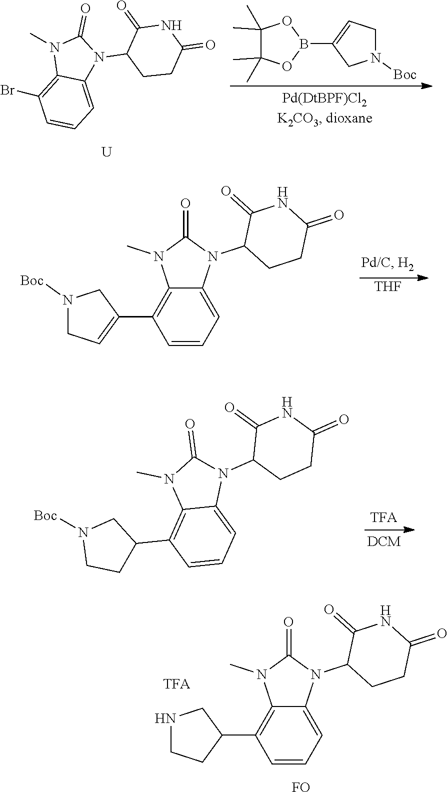

### Step 1—Tert-butyl 3-[1-(2,6-dioxopiperidin-3-yl)-3-methyl-2-oxo-1,3-benzodiazol-4-yl]-2,5-dihydropyrrole-1-carboxylate

To a stirred solution of 3-(4-bromo-3-methyl-2-oxo-1,3-benzodiazol-1-yl)piperidine-2,6-dione (600 mg, 2 mmol, Intermediate U) and tert-butyl 3-(4,4,5,5-tetramethyl-1,3,2-dioxaborolan-2-yl)-2,5-dihydropyrrole-1-carboxylate (628.51 mg, 2.129 mmol) in dioxane (15 mL) and H₂O (3 mL) were added K₂CO₃ (490.44 mg, 3.548 mmol) and Pd(dtbpf)Cl₂ (115.64 mg, 0.177 mmol) at 90° C. under nitrogen atmosphere. The resulting mixture was stirred for 1 h at 90° C. under nitrogen atmosphere. On completion, the reaction mixture was cooled rt and concentrated under reduced pressure. The residue was purified by reverse flash chromatography (column, C18 silica gel; mobile phase A: water (0.1% FA), mobile phase B: ACN, 35% to 55% gradient in 25 min; detector, UV 254 nm; the fractions were collected at 47%_ and concentrated under reduced pressure to afford the title compound (700 mg, 93% yield) as a yellow solid. LC/MS (ESI, m/z): [(M+H)]⁺=427.2.

### Step 2—Tert-butyl 3-[1-(2,6-dioxopiperidin-3-yl)-3-methyl-2-oxo-1,3-benzodiazol-4-yl]pyrrolidine-1-carboxylate

To a stirred solution of tert-butyl 3-[1-(2,6-dioxopiperidin-3-yl)-3-methyl-2-oxo-1,3-benzodiazol-4-yl]-2,5-dihydropyrrole-1-carboxylate (700 mg, 2 mmol) in THF (20 mL) was 10 wt % Pd/C (87.34 mg) under nitrogen atmosphere. The reaction system was degassed under vacuum and purged with H₂ several times, then was hydrogenated under H₂ balloon (1 atm) at 25° C. and stirred for 16 h. After completion of the reaction, Pd/C was filtered off through celite and the corresponding filtrate was concentrated under reduced pressure. The residue was purified by reverse flash chromatography (column, C18 silica gel; mobile phase A: water (0.1% FA), mobile phase B: ACN, 30% to 60% gradient in 30 min; detector, UV 254 nm; the fractions were collected at 42%) and concentrated under reduced pressure to afford the title compound (540 mg, 77% yield) as a yellow solid. LC/MS (ESI, m/z): [(M+H)]⁺=429.1.

### Step 3—3-[3-Methyl-2-oxo-4-(pyrrolidin-3-yl)-1,3-benzodiazol-1-yl]piperidine-2,6-dione Trifluoroacetate

To a solution of tert-butyl 3-[1-(2,6-dioxopiperidin-3-yl)-3-methyl-2-oxo-1,3-benzodiazol-4-yl]pyrrolidine-1-carboxylate (540 mg, 1.26 mmol) in DCM (15 mL) was added TFA (3 mL) dropwise at rt. The reaction mixture was stirred for 1 h at rt under nitrogen atmosphere. On completion, the reaction mixture was concentrated under reduced pressure. The residue was triturated with Et₂O to afford the title compound (400 mg) as a yellow solid. LC/MS (ESI, m/z): [(M+H)]⁺=329.1.

### Example 1.172. Preparation of tert-butyl 6-(4-bromophenyl)-2,6-diazaspiro[3.3]heptane-2-carboxylate (Intermediate FP)

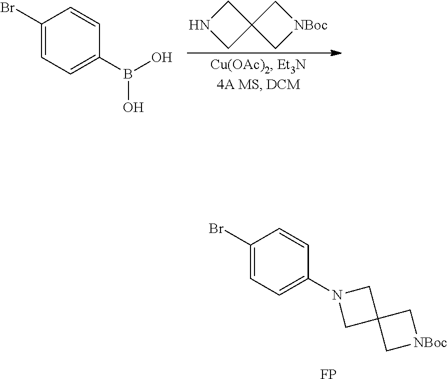 [SMILES: OB(O)c1ccc(Br)cc1 ; CC(C)(C)OC(=O)N1CC2(CNC2)C1 ; CC(C)(C)OC(=O)N1CC2(C1)CN(c1ccc(Br)cc1)C2 ; "FP"]

A mixture of (4-bromophenyl)boronic acid (2.51 g, 12.5 mmol), tert-butyl 2,6-diazaspiro[3.3]heptane-2-carboxylate (3 g, 10.4 mmol, oxalic acid), triethylamine (5.26 g, 52.0 mmol), copper acetate (2.84 g, 15.6 mmol) and 4 Å molecular sieves (6.8 g) in dichloromethane (200 mL) was degassed and purged with oxygen three times. Then the mixture was stirred at 25° C. for 12 hours under oxygen atmosphere. The reaction mixture was filtered and concentrated under reduced pressure to give a residue. The residue was purified by column chromatography (SiO₂, Petroleum ether/Ethyl acetate=20:1 to 0:1) to give the title compound (2.4 g, 65% yield) as an off-white solid. ¹H NMR (400 MHz, CHLOROFORM-d) δ=7.31 (d, J=8.8 Hz, 2H), 6.34 (d, J=8.8 Hz, 2H), 4.11 (s, 4H), 3.96 (s, 4H), 1.47 (s, 9H).

### Example 1.173. Preparation of 3-[5-(4-{2,6-diazaspiro[3.3]heptan-2-yl}phenyl)-3-methyl-2-oxo-1,3-benzodiazol-1-yl]piperidine-2,6-dione (Intermediate FQ)

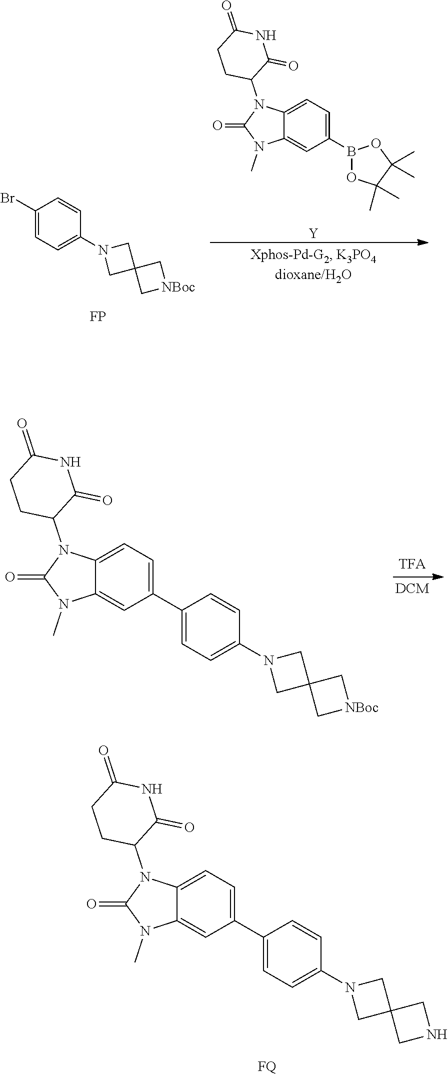

### Step 1—Tert-butyl 6-(4-(1-(2,6-dioxopiperidin-3-yl)-3-methyl-2-oxo-2,3-dihydro-1H-benzo[d]imidazol-5-yl)phenyl)-2,6-diazaspiro[3.3]heptane-2-carboxylate

A mixture of tert-butyl 6-(4-bromophenyl)-2,6-diazaspiro[3.3]heptane-2-carboxylate (2 g, 6 mmol, Intermediate FP), 3-[3-methyl-2-oxo-5-(4,4,5,5-tetramethyl-1,3,2-dioxaborolan-2-yl)benzimidazol-1-yl]piperidine-2,6-dione (2.18 g, 5.66 mmol, Intermediate Y), chloro(2-dicyclohexylphosphino-2′,4′,6′-triisopropyl-1,1′-biphenyl)[2-(2′-amino-1,1′-biphenyl)]palladium(II) (0.45 g, 0.57 mmol) and potassium phosphate (2.40 g, 11.3 mmol) in dioxane (40 mL) and water (4 mL) was degassed and purged with nitrogen three times. Then the mixture was stirred at 80° C. for 4 hrs under nitrogen atmosphere. On completion, the reaction mixture was then diluted with water (30 mL) and extracted with ethyl acetate (40 mL×2). The combined organic layers were washed with brine (50 mL×2), dried over anhydrous sodium sulfate, filtered and concentrated under reduced pressure to give a residue. The crude product was triturated with dimethylformamide (30 mL) at 25° C. for 30 min. Then the solid was washed with ethyl acetate (40 mL), petroleum ether (40 mL) to give the title compound (1.5 g, 46% yield) as an off-white solid. ¹H NMR (400 MHz, DMSO-d₆) δ=11.17-11.04 (m, 1H), 7.52 (d, J=8.4 Hz, 2H), 7.39 (s, 1H), 7.24 (d, J=7.6 Hz, 1H), 7.13 (d, J=8.0 Hz, 1H), 6.51 (d, J=8.4 Hz, 2H), 5.41-5.36 (m, 1H), 4.04 (s, 4H), 3.96 (s, 4H), 3.39 (s, 3H), 2.98-2.85 (m, 1H), 2.80-2.70 (m, 1H), 2.70-2.60 (m, 1H), 2.08-2.00 (m, 1H), 1.39 (s, 9H); LC-MS (ESI+) m/z 532.0 (M+H)⁺

### Step 2—3-[5-(4-{2,6-Diazaspiro[3.3]heptan-2-yl}phenyl)-3-methyl-2-oxo-1,3-benzodiazol-1-yl]piperidine-2,6-dione Trifluoroacetate

To a solution of tert-butyl 6-{4-[1-(2,6-dioxopiperidin-3-yl)-3-methyl-2-oxo-1,3-benzodiazol-5-yl]phenyl}-2,6-diazaspiro[3.3]heptane-2-carboxylate (50 mg, 0.094 mmol) in DCM (1.5 mL) was added TFA (0.3 mL) dropwise at rt. The reaction mixture was stirred for 2 h at rt under nitrogen atmosphere. On completion, the reaction mixture was concentrated under reduced pressure. The residue was triturated with Et₂O to afford the title compound (35 mg) as a yellow solid. LC/MS (ESI, m/z): [(M+H)]⁺=432.2.

### Example 1.174. Preparation of Tert-butyl 4-((1s,4s)-4-(4-(((trifluoromethyl)sulfonyl)oxy)phenyl)cyclohexyl)piperazine-1-carboxylate (Intermediate FR)

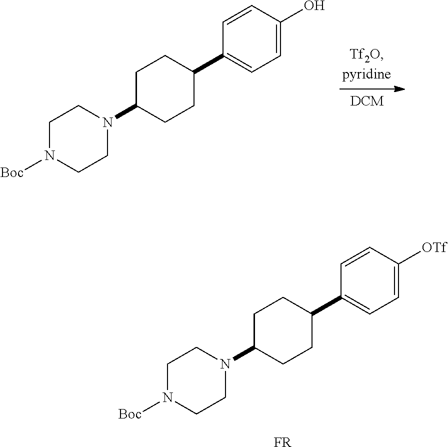 [SMILES: CC(C)(C)OC(=O)N1CCN([C@H]2CC[C@@H](c3ccc(O)cc3)CC2)CC1 ; CC(C)(C)OC(=O)N1CCN([C@H]2CC[C@@H](c3ccc(OS(=O)(=O)C(F)(F)F)cc3)CC2)CC1 ; "FR"]

To a solution of tert-butyl 4-((1s,4s)-4-(4-hydroxyphenyl)cyclohexyl)piperazine-1-carboxylate (4.10 g, 11.4 mmol, synthesized via Step 1 of Intermediate AB) in DCM (41 mL) was added pyridine (1.80 g, 22.8 mmol, 1.84 mL) and trifluoromethylsulfonyl trifluoromethanesulfonate (3.85 g, 13.7 mmol, 2.25 mL) under N₂ at 0° C. The mixture was stirred at 25° C. for 12 h. On completion, the reaction mixture was diluted with sat. NaHCO₃ (40 mL), and extracted with DCM (40 mL×3). The combined organic layers were washed with brine (20 mL×3), dried over Na₂SO₄ and evaporated to afford the title compound (5.60 g) as a yellow solid.

### Example 1.175. Preparation of 3-(3-methyl-2-oxo-5-{4-[(1s,4s)-4-(piperazin-1-yl)cyclohexyl]phenyl}-1,3-benzodiazol-1-yl)piperidine-2,6-dione (Intermediate FS)

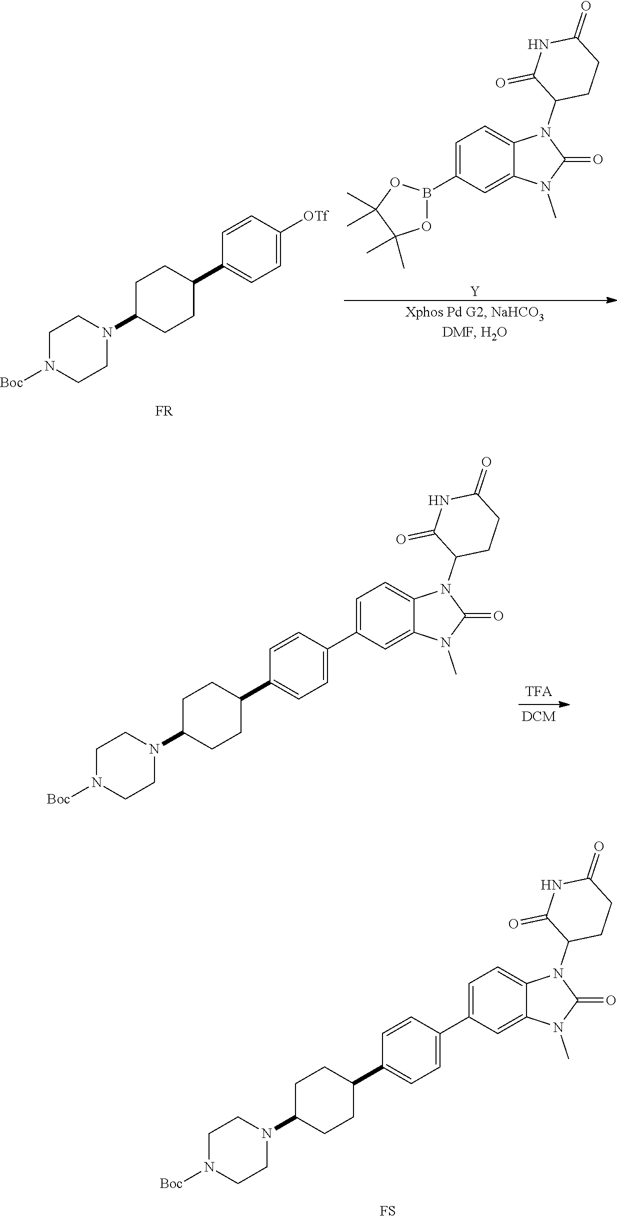

### Step 1—Tert-butyl 4-((1s,4s)-4-(4-(1-(2,6-dioxopiperidin-3-yl)-3-methyl-2-oxo-2,3-dihydro-1H-benzo[d]imidazol-5-yl)phenyl)cyclohexyl)piperazine-1-carboxylate

To a solution of tert-butyl 4-((1s,4s)-4-(4-(((trifluoromethyl)sulfonyl)oxy)phenyl)cyclohexyl)piperazine-1-carboxylate (1.54 g, 3.12 mmol, Intermediate FR), 3-(3-methyl-2-oxo-5-(4,4,5,5-tetramethyl-1,3,2-dioxaborolan-2-yl)-2,3-dihydro-1H-benzo[d]imidazol-1-yl)piperidine-2,6-dione (1.00 g, 2.60 mmol, Intermediate Y) and NaHCO₃ (437 mg, 5.20 mmol) in DMF (10 mL) and H₂O (2 mL) was added Xphos-Pd-G2 (102 mg, 130 umol) under N₂. The mixture was stirred at 80° C. for 4 h. On completion, the reaction mixture was filtered and the filtered cake was evaporated. The residue was purified by column chromatography (SiO₂, Petroleum ether/Ethyl acetate=0/1 to EA/DCM=1/1+0.1% TEA) to afford the title compound (1.10 g, 67% yield) as a white solid. LC-MS (ESI⁺) m/z 602.3 (M+H)⁺; ¹H NMR (400 MHz, DMSO-d₆) δ=11.12 (s, 1H), 7.61 (br d, J=8.0 Hz, 2H), 7.46 (s, 1H), 7.32 (br d, J=8.0 Hz, 3H), 7.18 (d, J=8.4 Hz, 1H), 5.40 (dd, J=5.2, 12.8 Hz, 1H), 3.41 (s, 3H), 3.34 (br s, 4H), 2.97-2.88 (m, 1H), 2.83-2.59 (m, 4H), 2.38 (br s, 2H), 2.23 (br s, 1H), 2.09-2.02 (m, 1H), 1.98-1.85 (m, 4H), 1.56 (br t, J=10.4 Hz, 4H), 1.41 (s, 9H).

### Step 2—3-(3-Methyl-2-oxo-5-{4-[(1s,4s)-4-(piperazin-1-yl)cyclohexyl]phenyl}-1,3-benzodiazol-1-yl)piperidine-2,6-dione Trifluoroacetate

To a solution of tert-butyl 4-[(1s,4s)-4-{4-[1-(2,6-dioxopiperidin-3-yl)-3-methyl-2-oxo-1,3-benzodiazol-5-yl]phenyl}cyclohexyl]piperazine-1-carboxylate (55 mg, 0.091 mmol) in DCM (2.5 mL) was added TFA (0.5 mL) dropwise at rt. The reaction mixture was stirred for 1 h at rt under nitrogen atmosphere. On completion, the reaction mixture was concentrated under reduced pressure. The residue was triturated with Et₂O to afford the title compound (52 mg) as a yellow solid. LC/MS (ESI, m/z): [(M+H)]⁺=502.2.

### Example 1.176. Preparation of Tert-butyl 4-[3-[2-(4-bromophenyl)ethoxy]propyl]piperazine-1-carboxylate (Intermediate FT)

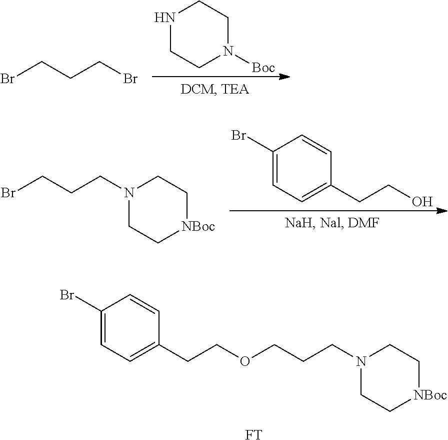 [SMILES: BrCCCBr ; CC(C)(C)OC(=O)N1CCNCC1 ; CC(C)(C)OC(=O)N1CCN(CCCBr)CC1 ; OCCc1ccc(Br)cc1 ; CC(C)(C)OC(=O)N1CCN(CCCOCCc2ccc(Br)cc2)CC1 ; "FT"]

### Step 1—tert-butyl 4-(3-bromopropyl)piperazine-1-carboxylate

To a solution of tert-butyl piperazine-1-carboxylate (13 g, 70 mmol) in dichloromethane (150 mL) was added triethylamine (7.77 g, 76.7 mmol) and 1,3-dibromopropane (28.1 g, 139 mmol) at 0° C. The mixture was stirred at 25° C. for 12 hrs. The reaction mixture was diluted with water (50 mL), and extracted with dichloromethane (50 mL×3), dried over anhydrous sodium sulfate, and concentrated under reduced pressure to give a residue. The crude was purified by silica gel column chromatography (Petroleum ether/Ethyl acetate=2:1) to give the title compound (4.5 g, 20% yield) as a colorless oil. ¹H NMR (400 MHz, DMSO-d₆) δ=3.54 (t, J=6.4 Hz, 2H), 3.31-3.27 (m, 4H), 2.39 (t, J=6.8 Hz, 2H), 2.33-2.25 (m, 4H), 1.99 (s, 2H), 1.41-1.36 (m, 9H).

### Step 2—tert-butyl 4-[3-[2-(4-bromophenyl)ethoxy]propyl]piperazine-1-carboxylate

To a slurry of 2-(4-bromophenyl)ethanol (2.94 g, 14.6 mmol) in dimethylformamide (60 mL) was added sodium hydrogen (1.17 g, 29.2 mmol, 60% dispersion in mineral oil) at 0° C., and the mixture was stirred for 1 hr at 25° C. Tert-butyl 4-(3-bromopropyl)piperazine-1-carboxylate (4.5 g, 14.6 mmol) and sodium iodide (219 mg, 1.46 mmol) was then added to the mixture at 0° C., and the mixture was stirred at 2 5° C. for 11 hrs. On completion, the reaction mixture was quenched by addition ammonium chloride (200 mL) at 0° C., and extracted with ethyl acetate (300 mL×3), dried over anhydrous sodium sulfate, and concentrated under reduced pressure to give a residue. The crude was purified by silica gel column chromatography (Petroleum ether/Ethyl acetate=1:1) to give the title compound (1.8 g, 27% yield) as a white solid. ¹H NMR (400 MHz, DMSO-d₆) δ=7.45 (t, J=5.6 Hz, 2H), 7.20 (d, J=4.0 Hz, 2H), 3.30-3.23 (m, 4H), 2.89 (s, 4H), 2.73 (s, 4H), 2.25-2.21 (m, 4H), 1.67-1.54 (m, 2H), 1.38 (s, 9H).

### Example 1.177. Preparation of 3-[3-methyl-2-oxo-5-(4-{2-[3-(piperazin-1-yl)propoxy]ethyl}phenyl)-1,3-benzodiazol-1-yl]piperidine-2,6-dione (Intermediate FU)

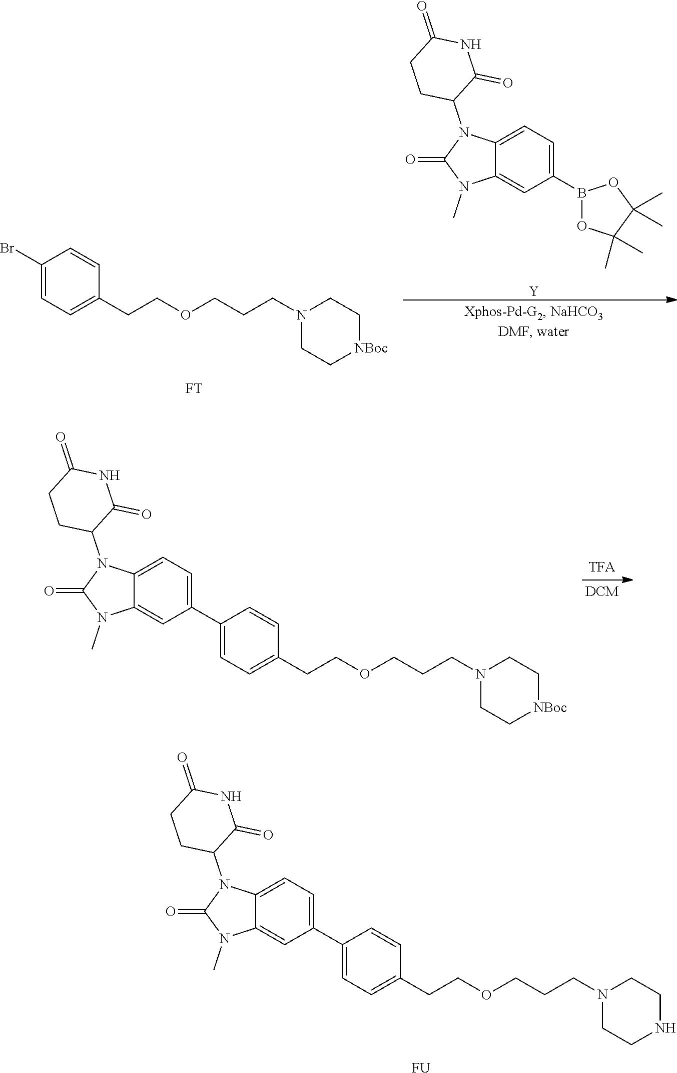

### Step 1—Tert-butyl 4-[3-[2-[4-[1-(2,6-dioxo-3-piperidyl)-3-methyl-2-oxo-benzimidazol-5-yl]phenyl]ethoxy]propyl]piperazine-1-carboxylate

A mixture of tert-butyl 4-[3-[2-(4-bromophenyl)ethoxy]propyl]piperazine-1-carboxylate (1.8 g, 4.21 mmol, Intermediate FT), 3-[3-methyl-2-oxo-5-(4,4,5,5-tetramethyl-1,3,2-dioxaborolan-2-yl)benzimidazol-1-yl]piperid-ine-2,6-dione (1.62 g, 4.21 mmol, Intermediate Y), chloro(2-dicyclohexylphosphino-2′,4′,6′-triisopropyl-1,1′-biphenyl)[2-(2′-amino-1,1′-biphenyl)]palladium(II) (331 mg, 421 umol), sodium bicarbonate (707 mg, 8.42 mmol) in water (4 mL) and dimethylformamide (40 mL) was degassed and purged with nitrogen three times. Then the mixture was stirred at 80° C. for 3 hrs under nitrogen atmosphere. On completion, the reaction mixture was diluted with water (50 mL), and extracted with ethyl acetate (100 mL×3), dried over anhydrous sodium sulfate, and concentrated under reduced pressure to give a residue. The residue was purified by perp-HPLC (column: Phenomenex luna C18 150*40 mm*15 um; mobile phase: [water (FA)-ACN]; B %: 13%-43%, 10 min) to give the title compound (0.977 g, 35% yield) as a white solid. ¹H NMR (400 MHz, DMSO-d₆) δ=11.15-11.07 (m, 1H), 7.60 (d, J=8.0 Hz, 2H), 7.47 (d, J=1.6 Hz, 1H), 7.34-7.28 (m, 3H), 7.18 (d, J=8.4 Hz, 1H), 5.39 (m, J=12.8, 5.50 Hz, 1H), 3.59 (s, 2H), 3.40 (s, 3H), 3.27 (d, J=4.4 Hz, 4H), 2.83 (t, J=6.8 Hz, 2H), 2.70-2.58 (m, 2H), 2.34-2.18 (m, 8H), 2.09-1.95 (m, 2H), 1.66-1.60 (m, 2H), 1.37 (s, 9H); LCMS=606.4[M+H]⁺.

### Step 2—3-[3-methyl-2-oxo-5-(4-{2-[3-(piperazin-1-yl)propoxy]ethyl}phenyl)-1,3-benzodiazol-1-yl]piperidine-2,6-dione Trifluoroacetate

To a stirred solution of tert-butyl 4-[3-(2-{4-[1-(2,6-dioxopiperidin-3-yl)-3-methyl-2-oxo-1,3-benzodiazol-5-yl]phenyl}ethoxy)propyl]piperazine-1-carboxylate (50 mg, 0.083 mmol) in DCM (1 mL) was added TFA (0.2 mL) dropwise at rt. The resulting mixture was stirred for 1 h at rt under nitrogen atmosphere. On completion, the reaction mixture was concentrated under reduced pressure. The residue was purified by trituration with Et₂O to give the title compound (60 mg) as a white solid. LC/MS (ESI, m/z): [(M+1)]⁺=506.3.

### Example 1.178. Preparation of tert-butyl 4-(5-bromopyrimidin-2-yl)-5,6-dihydropyridine-1(2H)-carboxylate (Intermediate FV)

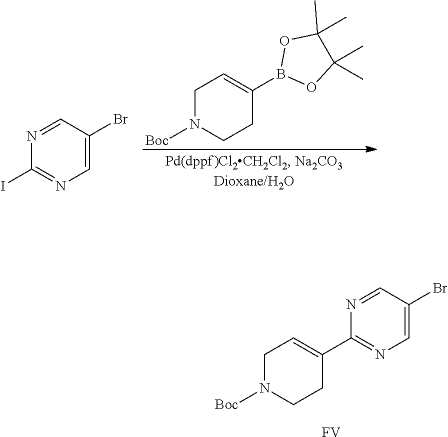

A mixture of 5-bromo-2-iodopyrimidine (20 g, 70 mmol), tert-butyl 4-(4,4,5,5-tetramethyl-1,3,2-dioxaborolan-2-yl)-5,6-dihydropyridine-1(2H)-carboxylate (20.6 g, 66.7 mmol, CAS #286961-14-6), Na₂CO₃ (22.3 g, 210 mmol) and Pd(dppf)Cl₂·CH₂Cl₂ (2.87 g, 3.51 mmol) in dioxane (200 mL) and H₂O (20 mL), then the mixture was stirred at 60° C. for 12 h under N₂ atmosphere. On completion, the reaction was filtered and quenched by addition of H₂O (200 mL) at 20° C., then extracted with EtOAc (200 mL×3). The combined organic layers were washed with brine (200 mL×2), dried over Na₂SO₄, filtered and concentrated under reduced pressure to give a residue. The residue was purified by column chromatography (SiO₂, Petroleum ether/Ethyl acetate=10/1 to 3/1) to give the title compound (4 g, 18% yield) as yellow solid. ¹H NMR (400 MHz, DMSO-d₆) δ=8.95 (s, 2H), 7.18 (br s, 1H), 4.08 (br s, 2H), 3.52 (t, J=5.6 Hz, 2H), 2.57 (br d, J=2.0 Hz, 2H), 1.42 (s, 9H).

### Example 1.179. Preparation of 3-{3-methyl-2-oxo-5-[2-(piperidin-4-yl)pyrimidin-5-yl]-1,3-benzodiazol-1-yl}piperidine-2,6-dione (Intermediate FW)

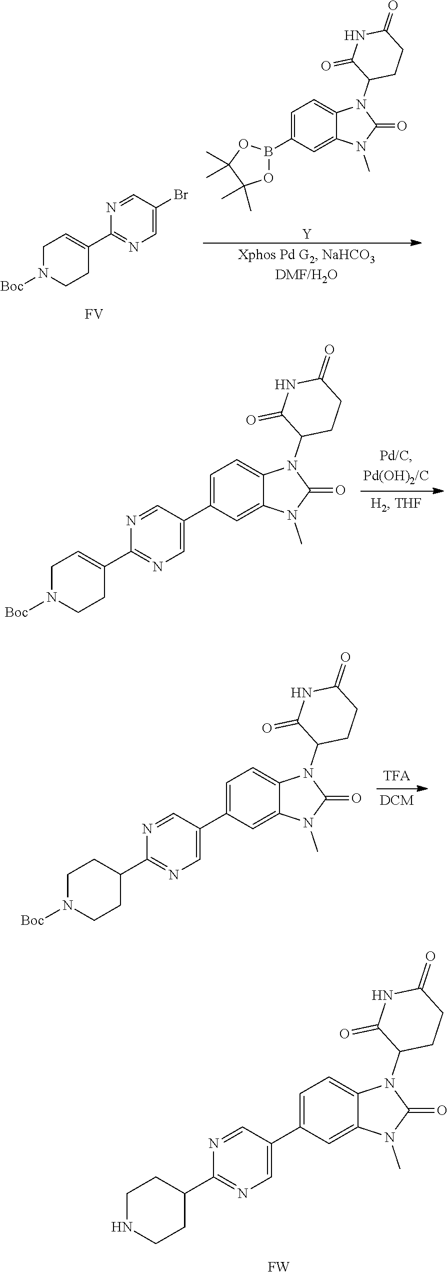

### Step 1—Tert-butyl 4-(5-(1-(2,6-dioxopiperidin-3-yl)-3-methyl-2-oxo-2,3-dihydro-1H-benzo[d] imidazol-5-yl)pyrimidin-2-yl)-5,6-dihydropyridine-1(2H)-carboxylate

A mixture of tert-butyl 4-(5-bromopyrimidin-2-yl)-5,6-dihydropyridine-1(2H)-carboxylate (4.4 g, 12.9 mmol, Intermediate FV), 3-(3-methyl-2-oxo-5-(4,4,5,5-tetramethyl-1,3,2-dioxaborolan-2-yl)-2,3-dihydro-1H-benzo[d]imidazol-1-yl)piperidine-2,6-dione (4.98 g, 12.9 mmol, Intermediate Y), XPhos Pd G2 (1.02 g, 1.29 mmol) and NaHCO₃ (2.17 g, 25.8 mmol, 1.01 mL) in DMF (40 mL) and H₂O (4 mL) was stirred at 80° C. for 2 h. On completion, the reaction mixture was quenched with H₂O (40 mL) at 20° C., and extracted with EtOAc (40 mL×3). The combined organic layers were washed with brine (40 mL×2), dried over Na2SO4, filtered and concentrated under reduced pressure to give a residue. The residue was purified by column chromatography (SiO₂, Petroleum ether/Ethyl acetate=5/1 to 0/1) to give the title compound ((1.27 g, 19% yield) as yellow solid. ¹H NMR (400 MHz, DMSO-d₆) δ=11.13 (s, 1H), 9.14 (s, 2H), 7.70 (d, J=1.6 Hz, 1H), 7.51 (dd, J=1.6, 8.4 Hz, 1H), 7.43-7.34 (m, 1H), 7.30-7.10 (m, 3H), 5.46-5.38 (m, 2H), 3.42 (s, 3H), 2.98-2.86 (m, 2H), 2.84-2.72 (m, 1H), 2.66 (br s, 4H), 1.44 (s, 10H).

### Step 2—Tert-butyl 4-(5-(1-(2,6-dioxopiperidin-3-yl)-3-methyl-2-oxo-2,3-dihydro-1H-benzo[d] imidazol-5-yl)pyrimidin-2-yl)piperidine-1-carboxylate

A solution of tert-butyl 4-(5-(1-(2,6-dioxopiperidin-3-yl)-3-methyl-2-oxo-2,3-dihydro-1H-benzo[d]imidazol-5-yl)pyrimidin-2-yl)-5,6-dihydropyridine-1(2H)-carboxylate (1.27 g, 2.45 mmol), Pd/C (200 mg, 188 umol, 10 wt %) and Pd(OH)₂/C (500 mg, 712 umol, 20 wt %) in THF (15 mL), then the mixture was stirred at 25° C. for 12 h under H₂. On completion, the mixture was filtered and concentrated in vacuo. The residue was purified by column chromatography (SiO₂, Petroleum ether/Ethyl acetate=3/1 to 0/1) to give the title compound (600 mg, 47% yield) as white solid. ¹H NMR (400 MHz, DMSO-d₆) δ=11.10 (br s, 1H), 9.09 (s, 2H), 7.67 (d, J=1.6 Hz, 1H), 7.47 (dd, J=1.6, 8.0 Hz, 1H), 7.27 (d, J=8.4 Hz, 1H), 5.42 (dd, J=5.2, 12.8 Hz, 1H), 4.04 (br d, J=11.6 Hz, 2H), 3.41 (s, 3H), 3.11-3.01 (m, 1H), 3.00-2.85 (m, 3H), 2.76 (br dd, J=4.4, 13.2 Hz, 1H), 2.67-2.61 (m, 1H), 2.08-1.93 (m, 3H), 1.67 (dq, J=4.4, 12.4 Hz, 2H), 1.42 (s, 9H).

### Step 3—3-{3-Methyl-2-oxo-5-[2-(piperidin-4-yl)pyrimidin-5-yl]-1,3-benzodiazol-1-yl}piperidine-2,6-dione Trifluoroacetate

To a solution of tert-butyl 4-{5-[1-(2,6-dioxopiperidin-3-yl)-3-methyl-2-oxo-1,3-benzodiazol-5-yl]pyrimidin-2-yl}piperidine-1-carboxylate (50 mg, 0.1 mmol) in DCM (1.5 mL) was added TFA (0.3 mL) dropwise at rt. The reaction solution was stirred for 2 h at rt under nitrogen atmosphere. On completion, the reaction mixture was concentrated under reduced pressure. The residue was triturated with Et₂O to afford the title compound (35 mg) as a white solid. LC/MS (ESI, m/z): [(M+H)]⁺=421.1.

### Example 1.180. Preparation of tert-butyl ((1r,4r)-4-(2-(prop-2-yn-1-yl)-2,6-diazaspiro[3.4]octane-6-carbonyl)cyclohexyl) carbamate (Intermediate FX)

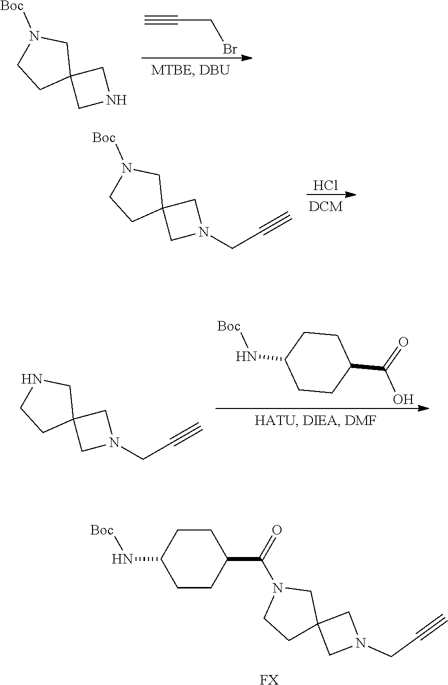

### Step 1—2-Tert-butyl 2-(prop-2-yn-1-yl)-2,6-diazaspiro[3.4]octane-6-carboxylate

To a solution of tert-butyl 2, 6-diazaspiro[3.4]octane-6-carboxylate (3.7 g, 17 mmol, CAS #885270-86-0) in MTBE (37 mL) was added DBU (3.98 g, 26.14 mmol, 3.94 mL) at 0° C. Then 3-bromoprop-1-yne (2.49 g, 20.91 mmol, 1.80 mL) was added, and the mixture was stirred at 25° C. for 16 hrs. On completion, the reaction mixture was diluted with H₂O (150 mL) and extracted with EA (100 mL×3). The combined organic layers were washed with brine (100 mL), dried over Na₂SO₄, and filtered and concentrated under reduced pressure to give a residue. The residue was purified by column chromatography (SiO₂, Petroleum ether/Ethyl acetate=100/1 to 0/1) to give the title compound (3.8 g, 87% yield) as a yellow oil. ¹H NMR (400 MHz, CHLOROFORM-d) δ ppm 3.42 (s, 2H) 3.24-3.39 (m, 8H) 2.27 (s, 1H) 1.94-2.11 (m, 2H) 1.45 (s, 9H).

### Step 2—2-(Prop-2-yn-1-yl)-2,6-diazaspiro[3.4]octane

To a solution of tert-butyl 2-(prop-2-yn-1-yl)-2, 6-diazaspiro[3.4]octane-6-carboxylate (3.8 g, 15 mmol) in DCM (30 mL) was added HCl (4 M, 20 mL). The mixture was stirred at 25° C. for 1.5 hr. On completion, the mixture was concentrated to give the title compound (3.6 g, HCl) as a yellow solid. ¹H NMR (400 MHz, DMSO-d6) δ ppm 11.91-12.23 (m, 1H) 9.50-9.82 (m, 2H) 3.93-4.24 (m, 7H) 3.77 (s, 1H) 3.30-3.37 (m, 1H) 3.16 (brs, 2H) 2.14-2.33 (m, 2H).

### Step 3—Tert-butyl ((1r,4r)-4-(2-(prop-2-yn-1-yl)-2,6-diazaspiro[3.4]octane-6-carbonyl)cyclohexyl) carbamate

To a solution of 4-(tert-butoxycarbonylamino) cyclohexanecarboxylic acid (2.61 g, 10.7 mmol) in DMF (30 mL) was added HATU (6.11 g, 16.1 mmol) and DIEA (8.31 g, 64.28 mmol, 11.20 mL). The mixture was stirred at 25° C. for 0.5 hr. Then 2-(prop-2-yn-1-yl)-2, 6-diazaspiro[3.4]octane (3.2 g, 17.14 mmol, HCl) was added, then the mixture was stirred at 25° C. for 1.5 hrs. On completion, the reaction mixture ws diluted with H₂O (150 mL) and extracted with EA (150 mL×4). The combined organic layers were washed with brine (150 mL), dried over Na₂SO₄, filtered and concentrated under reduced pressure to give a residue. The residue was purified by column chromatography (SiO₂, EA/EtOH=100/1 to 1/1, TLC (EA/EtOH=2:1, Rf=0.2, KMnO₄)) to give the title compound (5 g, quant. yield) as a brown gum. ¹H NMR (400 MHz, CDCl₃-d) δ ppm 4.49-4.31 (m, 1H), 3.64 (s, 1H), 3.55 (s, 1H), 3.53-3.29 (m, 8H), 2.36-2.30 (m, 1H), 2.29-2.16 (m, 2H), 2.15-2.06 (m, 2H), 2.00 (t, J=7.6 Hz, 1H), 1.88-1.73 (m, 2H), 1.72-1.55 (m, 2H), 1.44 (d, J=1.6 Hz, 9H), 1.18-1.01 (m, 2H).

### Example 1.181. Preparation of 3-(5-{3-[6-(4-aminocyclohexanecarbonyl)-2,6-diazaspiro[3.4]octan-2-yl]prop-1-yn-1-yl}-3-methyl-2-oxo-1,3-benzodiazol-1-yl)piperidine-2,6-dione (Intermediate FY)

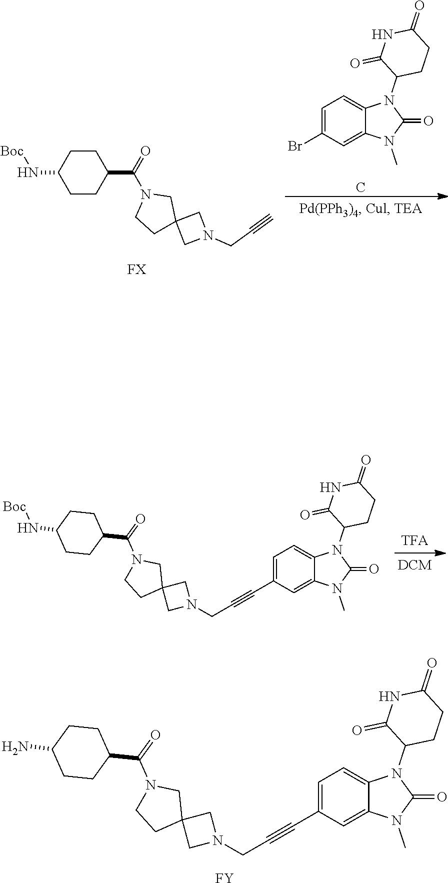

### Step 1—Tert-butyl ((1r,4r)-4-(2-(3-(1-(2,6-dioxopiperidin-3-yl)-3-methyl-2-oxo-2,3-dihydro-1H-benzo[d]imidazol-5-yl)prop-2-yn-1-yl)-2,6-diazaspiro[3.4]octane-6-carbonyl)cyclohexyl)carbamate

To a solution of 3-(5-bromo-3-methyl-2-oxo-2, 3-dihydro-1H-benzo[d]imidazol-1-yl) piperidine-2, 6-dione (3 g, 9 mmol, Intermediate C) in DMSO (45 mL) was added Pd(PPh₃)₄ (1.03 g, 887 μmol) and CuI (168.96 mg, 887.16 μmol) TEA (2.69 g, 26.61 mmol, 3.70 mL). The mixture was stirred at 25° C. for 0.5 hrs. Then tert-butyl N-[4-(2-prop-2-ynyl-2, 7-diazaspiro[3.4] octane-7-carbonyl) cyclohexyl]carbamate (3.66 g, 9.76 mmol, Intermediate FX) was added, then the mixture was stirred at 90° C. for 2.5 hrs. On completion, the mixture was diluted with H₂O (200 mL) and extracted with EA (100 mL×4). The combined organic layers were washed with brine (200 mL), dried over Na₂SO₄, filtered and concentrated under reduced pressure to give a residue. The residue was purified by reversed-phase HPLC (FA condition) twice to give a crude product. The crude product was further purified by Prep-HPLC (column: Phenomenex luna C18 150*40 mm*15 um; mobile phase: [water (FA)-ACN]; B %: 8%-38%, 10 min) to give the title compound (1.38 g, 25% yield) as an off-white solid. ¹H NMR (400 MHz, DMSO-d6) δ ppm 11.13 (s, 1H) 8.15 (s, 1H) 7.31 (s, 1H) 7.08-7.21 (m, 2H) 6.74 (d, J=7.6 Hz, 1H) 5.39 (dd, J=12.4, 5.2 Hz, 1H) 3.59 (s, 1H) 3.45-3.50 (m, 3H) 3.36-3.40 (m, 1H) 3.09-3.29 (m, 7H) 2.83-2.95 (m, 1H) 2.58-2.76 (m, 2H) 2.19-2.29 (m, 1H) 1.97-2.09 (m, 2H) 1.93 (t, J=7.2 Hz, 1H) 1.78 (d, J=10.4 Hz, 2H) 1.67 (d, J=12.8 Hz, 2H) 1.37 (d, J=2.0 Hz, 9H) 1.25-1.36 (m, 3H) 1.10-1.20 (m, 2H).

### Step 2—3-(5-{3-[6-(4-Aminocyclohexanecarbonyl)-2,6-diazaspiro[3.4]octan-2-yl]prop-1-yn-1-yl}-3-methyl-2-oxo-1,3-benzodiazol-1-yl)piperidine-2,6-dione trifluoroacetate

To a stirred solution of tert-butyl N-[4-(2-{3-[1-(2,6-dioxopiperidin-3-yl)-3-methyl-2-oxo-1,3-benzodiazol-5-yl]prop-2-yn-1-yl}-2,6-diazaspiro[3.4]octane-6-carbonyl)cyclohexyl]carbamate (50 mg, 0.08 mmol) in DCM (1.5 mL) was added TFA (0.5 mL) dropwise at rt under nitrogen atmosphere. The resulting mixture was stirred for 1 h at rt under nitrogen atmosphere. On completion, the reaction mixture was concentrated under reduced pressure. The residue was purified by trituration with ethyl ether (10 mL). The precipitated solids were collected by filtration and washed with ethyl ether (3×5 mL) to afford the title compound (50 mg) as a white solid. LC/MS (ESI, m/z): [(M+1)]⁺=533.3.

### Example 1.182. Preparation of 3-(3-methyl-2-oxo-5-((5-(1-oxo-2,7-diazaspiro[4.4]nonan-2-yl)pyrimidin-2-yl)ethynyl)-2,3-dihydro-1H-benzo[d]imidazol-1-yl)piperidine-2,6-dione (Intermediate FZ)

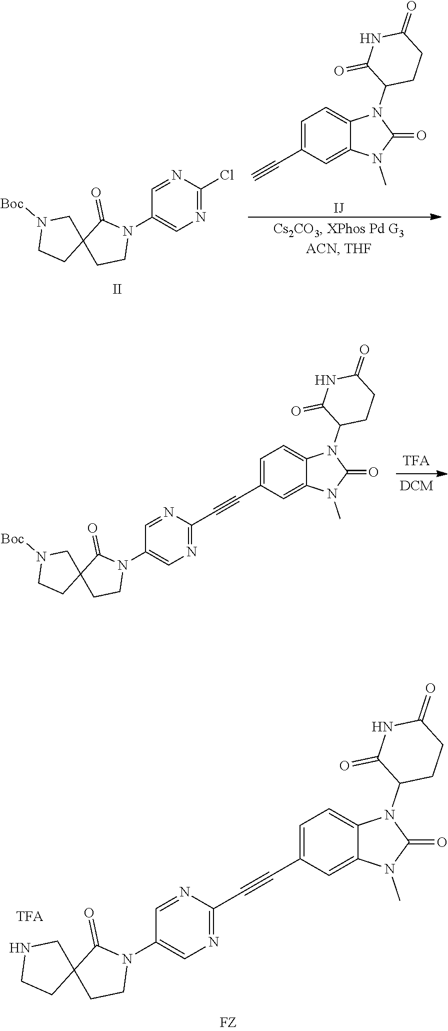

### Step 1—Tert-butyl 7-(2-((1-(2,6-dioxopiperidin-3-yl)-3-methyl-2-oxo-2,3-dihydro-1H-benzo[d]imidazol-5-yl)ethynyl)pyrimidin-5-yl)-6-oxo-2,7-diazaspiro[4.4]nonane-2-carboxylate

To a solution of tert-butyl 7-(2-chloropyrimidin-5-yl)-6-oxo-2,7-diazaspiro[4.4]nonane-2-carboxylate (1.70 g, 4.81 mmol, Intermediate II) and 3-(5-ethynyl-3-methyl-2-oxo-2,3-dihydro-1H-benzo[d]imidazol-1-yl)piperidine-2,6-dione (1.5 g, 5.30 mmol, Intermediate IJ) in THF (15 mL) and ACN (15 mL) was added XPhos Pd G3 (204 mg, 241 umol) and Cs₂CO₃ (4.71 g, 14.4 mmol) under N₂. The mixture was then stirred at 60° C. for 20 h. On completion, the reaction mixture was quenched with NH₄Cl (20 mL) and extracted with EtOAc (40 mL×3). The combined organic layers were washed with brine (20 mL×3), dried over Na₂SO₄ and evaporated. The residue was column chromatography (Petroleum ether/Ethyl acetate=7:1˜0:1) to give the title compound (990 mg, 34% yield) as a white solid. ¹H NMR (400 MHz, DMSO-d₆) δ 11.14 (s, 1H), 9.18 (s, 2H), 7.56 (d, J=1.2 Hz, 1H), 7.38 (dd, J=1.2, 8.2 Hz, 1H), 7.24 (d, J=8.2 Hz, 1H), 5.43 (dd, J=5.2, 12.8 Hz, 1H), 3.92 (br t, J=6.8 Hz, 2H), 3.48 (br d, J=10.0 Hz, 2H), 3.39 (s, 3H), 3.35 (br s, 1H), 2.95-2.84 (m, 1H), 2.79-2.59 (m, 3H), 2.24-2.15 (m, 3H), 2.13-2.03 (m, 2H), 2.00-1.90 (m, 1H), 1.41 (s, 9H), 1.35 (s, 2H).

### Step 2—3-(3-Methyl-2-oxo-5-((5-(1-oxo-2,7-diazaspiro[4.4]nonan-2-yl)pyrimidin-2-yl)ethynyl)-2,3-dihydro-1H-benzo[d]imidazol-1-yl)piperidine-2,6-dione

To a solution of tert-butyl 7-(2-((1-(2,6-dioxopiperidin-3-yl)-3-methyl-2-oxo-2,3-dihydro-1H-benzo[d]imidazol-5-yl)ethynyl)pyrimidin-5-yl)-6-oxo-2,7-diazaspiro[4.4]nonane-2-carboxylate (1.25 g, 2.08 mmol) in DCM (5 mL) was added TFA (1 mL). The mixture was stirred at 25° C. for 7 h. On completion, the mixture was concentrated. The crude product was purified by reversed-phase HPLC (water (FA)-ACN]; B %: 5%-35%, 10 min) to give the title compound (733.58 mg, 70% yield) as a white solid. LC-MS (ESI⁺) m/z 500.1 (M+H)⁺. ¹H NMR (400 MHz, DMSO-d₆) δ 9.18 (s, 2H), 7.54 (d, J=1.2 Hz, 1H), 7.38 (dd, J=1.2 8.2 Hz, 1H), 7.24 (d, J=8.2 Hz, 1H), 5.43 (dd, J=5.2 12.8 Hz, 1H), 3.82-3.79 (m, 2H), 3.39 (s, 3H), 3.23-3.21 (m, 2H), 2.95-2.85 (m, 1H), 2.79-2.60 (m, 3H), 2.32-2.20 (m, 2H), 2.20-2.09 (m, 2H), 2.08 (br s, 3H). ¹H NMR (400 MHz, DMSO-d₆) δ 1.15 (br s, 1H), 9.19 (s, 2H), 7.55 (d, J=1.0 Hz, 1H), 7.38 (dd, J=1.2, 8.2 Hz, 1H), 7.24 (d, J=8.4 Hz, 1H), 5.43 (dd, J=5.2 12.8 Hz, 1H), 3.94 (t, J=6.8 Hz, 2H), 3.39 (s, 3H), 3.34 (br s, 2H), 2.94-2.86 (m, 1H), 2.79-2.62 (m, 3H), 2.36-2.24 (m, 3H), 2.24-2.13 (m, 2H), 2.07 (td, J=6.8, 13.2 Hz, 2H).

### Example 1.183. Preparation of 3-(5-(4-((5-oxa-2-azaspiro[3.4]octan-7-yl)methyl)piperazin-1-yl)-3-methyl-2-oxo-2,3-dihydro-1H-benzo[d]imidazol-1-yl)piperidine-2,6-dione (Intermediate GA)

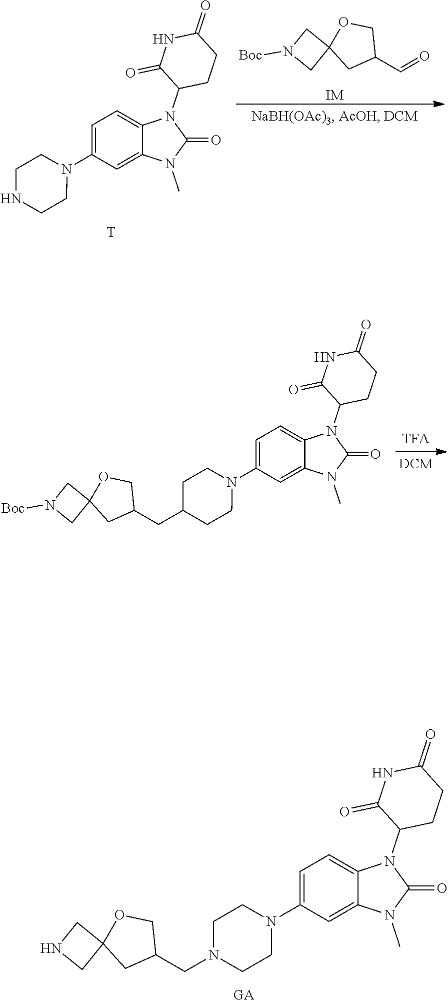

### Step 1—Tert-butyl 7-((4-(1-(2,6-dioxopiperidin-3-yl)-3-methyl-2-oxo-2,3-dihydro-1H-benzo[d]imidazol-5-yl)piperazin-1-yl)methyl)-5-oxa-2-azaspiro[3.4]octane-2-carboxylate

To a solution of tert-butyl 7-formyl-5-oxa-2-azaspiro[3.4]octane-2-carboxylate (543 mg, 2.25 mmol, Intermediate IM) and 3-(3-methyl-2-oxo-5-(piperazin-1-yl)-2,3-dihydro-1H-benzo[d]imidazol-1-yl)piperidine-2,6-dione (774 mg, 2.25 mmol, Intermediate T) in DCM (30 mL) was added AcOH (406 mg, 6.76 mmol) and NaBH(OAc)₃ (1.43 g, 6.76 mmol) at 20° C. under nitrogen flow. Then the reaction was stirred at 20° C. for 10 h under nitrogen atmosphere. On completion, the reaction was poured into NaHCO₃ (sat.aq, 40 mL) and extracted with DCM (50 mL×2). The combined organic phase is washed with brine (30 mL×2), and dried over Na₂SO₄. After filtration, the filtrate was concentrated to give a residue. The residue was purified by column chromatography on silica gel (eluted with dichloromethane:ethyl acetate=100:1 to 100:50) to give the title compound (947.62 mg, 55% yield) as a pink solid. LC-MS (ESI⁺) m/z 569.3 (M+H)⁺; ¹H NMR (400 MHz, DMSO-d₆) δ=11.06 (s, 1H), 6.94 (d, J=8.8 Hz, 1H), 6.84 (d, J=2.0 Hz, 1H), 6.63 (dd, J=2.0, 8.8 Hz, 1H), 5.29 (dd, J=5.6, 12.8 Hz, 1H), 3.98-3.81 (m, 5H), 3.80-3.75 (m, 1H), 3.50 (dd, J=6.4, 8.4 Hz, 1H), 3.31 (s, 3H), 3.08 (br s, 4H), 3.00-2.82 (m, 2H), 2.77-2.55 (m, 5H), 2.36-2.28 (m, 2H), 2.23 (br dd, J=7.6, 12.8 Hz, 1H), 2.03-1.95 (m, 1H), 1.81 (dd, J=7.2, 12.8 Hz, 1H), 1.38 (s, 9H).

### Step 2—3-(5-(4-(5-oxa-2-azaspiro[3.4]octan-7-ylmethyl)piperazin-1-yl)-3-methyl-2-oxo-2,3-dihydro-1H-benzo[d]imidazol-1-yl)piperidine-2,6-dione

To a solution of tert-butyl 7-((4-(1-(2,6-dioxopiperidin-3-yl)-3-methyl-2-oxo-2,3-dihydro-1H-benzo[d]imidazol-5-yl)piperazin-1-yl)methyl)-5-oxa-2-azaspiro[3.4]octane-2-carboxylate (200 mg, 351 umol) in DCM (5 mL) was added TFA (1.54 g, 13.5 mmol) at 20° C. under nitrogen flow. Then the reaction was stirred at 20° C. for 10 h under nitrogen atmosphere. On completion, the reaction was concentrated to give a residue. The residue was purified by prep-HPLC (column: Phenomenex Luna C18 150*25 mm*10 um; mobile phase: [water (FA)-ACN]; B %: 0%-12%, 10 min) to give the title compound (56.1 mg, 33% yield) as yellow solid. LC-MS (ESI⁺) m/z 469.4 (M+H)⁺; ¹H NMR (400 MHz, DMSO-d₆) δ=11.07 (s, 1H), 8.81 (br d, J=4.8 Hz, 2H), 6.98 (br d, J=8.8 Hz, 1H), 6.90 (br s, 1H), 6.68 (br d, J=8.0 Hz, 1H), 5.31 (dd, J=5.6, 12.8 Hz, 1H), 4.08-4.03 (m, 2H), 4.00-3.90 (m, 2H), 3.60-3.50 (m, 2H), 3.32 (br s, 3H), 3.18-2.97 (m, 4H), 2.96-2.84 (m, 2H), 2.77-2.59 (m, 4H), 2.54-2.52 (m, 5H), 2.04-1.96 (m, 1H), 1.92 (dd, J=7.6, 13.2 Hz, 1H).

### Example 1.184. Preparation of Tert-butyl N-[(3R)-1-(4-bromophenyl)pyrrolidin-3-yl]-N-methyl-carbamate (Intermediate GC)

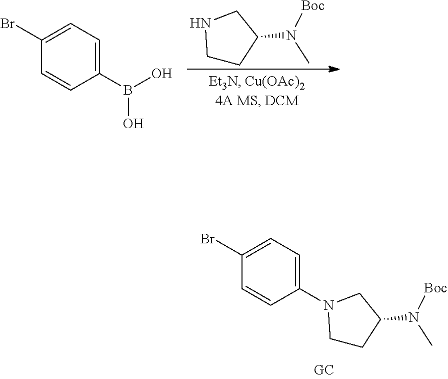 [SMILES: OB(O)c1ccc(Br)cc1 ; CN(C(=O)OC(C)(C)C)[C@@H]1CCNC1 ; CN(C(=O)OC(C)(C)C)[C@@H]1CCN(c2ccc(Br)cc2)C1]

A mixture of (4-bromophenyl)boronic acid (7.52 g, 37.4 mmol, CAS #5467-74-3), tert-butyl N-methyl-N-[(3R)-pyrrolidin-3-yl]carbamate (5.00 g, 24.9 mmol, CAS #392338-15-7), triethylamine (12.6 g, 124 mmol, 17.3 mL), copper acetate (6.80 g, 37.4 mmol) and 4 Å molecular sieve (1 g) in dichloromethane (200 mL) was degassed and purged with oxygen three times. Then the mixture was stirred at 25° C. for 12 hours under oxygen atmosphere. On completion, the mixture was filtered through celite, then the filter cake was discarded and the filtrate was concentrated in vacuo to afford a residue. The residue was purified by flash silica gel chromatography (TLC:PE:EA 3:1) to give the title compound (4.00 g, 45% yield) as a white solid. ¹H NMR (400 MHz, CHLOROFORM-d) δ=7.34 (d, J=9.2 Hz, 2H), 6.50 (s, 2H), 5.03-4.80 (m, 1H), 3.52-3.41 (m, 2H), 3.32-3.19 (m, 2H), 2.83 (s, 3H), 2.31-2.09 (m, 2H), 1.50 (s, 9H).

### Example 1.185. Preparation of 3-(3-methyl-5-{4-[(3R)-3-(methylamino)pyrrolidin-1-yl]phenyl}-2-oxo-1,3-benzodiazol-1-yl)piperidine-2,6-dione (Intermediate GD)

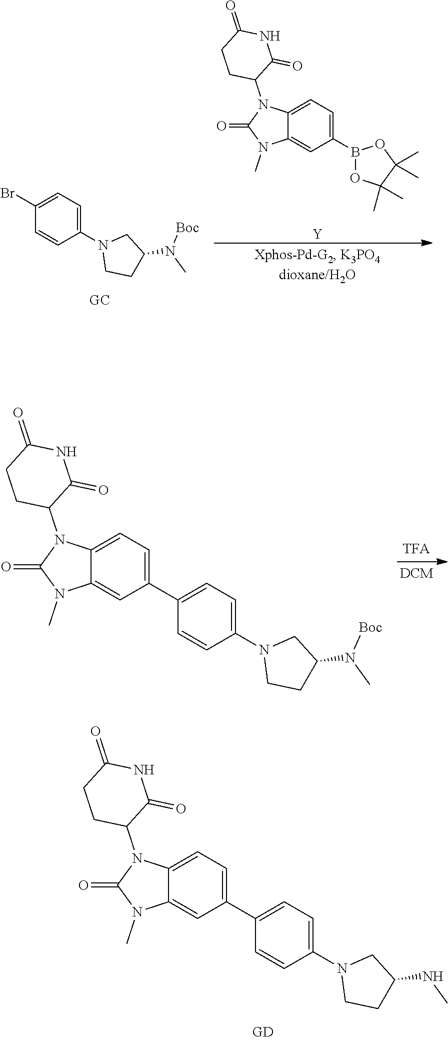

### Step 1—Tert-butyl 4-(3-(4-bromophenyl)propioloyl)piperazine-1-carboxylate

A mixture of tert-butyl N-[(3R)-1-(4-bromophenyl)pyrrolidin-3-yl]-N-methyl-carbamate (3.84 g, 10.8 mmol, Intermediate GC), 3-[3-methyl-2-oxo-5-(4,4,5,5-tetramethyl-1,3,2-dioxaborolan-2-yl)benzimidazol-1-yl]piperidine-2,6-dione (3.20 g, 8.31 mmol, Intermediate Y) chloro(2-dicyclohexylphosphino-2′,4′,6′-triisopropyl-1,1′-biphenyl)[2-(2′-amino-1,1′-biphenyl)]palladium(II) (653 mg, 830 umol) and potassium phosphate (5.29 g, 24.92 mmol) in dioxane (60 mL) and water (6 mL) was degassed and purged with nitrogen three times. Then the mixture was stirred at 80° C. for 4 hours under nitrogen atmosphere. On completion, the reaction mixture was quenched with water (150 mL) at 25° C., and extracted with ethyl acetate (200 mL×3). The combined organic layers were washed with brine (200 mL), dried over anhydrous sodium sulfate, filtered and concentrated. The residue was purified by reversed-phase HPLC (0.1% FA condition) to give the title compound (868.4 mg, 18% yield) as a yellow solid. ¹H NMR (400 MHz, DMSO-d6) δ=11.11 (s, 1H), 7.53 (d, J=8.4 Hz, 2H), 7.39 (d, J=1.2 Hz, 1H), 7.25 (dd, J=1.6, 8.4 Hz, 1H), 7.11 (d, J=8.4 Hz, 1H), 6.65 (d, J=8.8 Hz, 2H), 5.37 (dd, J=5.6, 12.8 Hz, 1H), 4.89-4.61 (m, 1H), 3.49-3.42 (m, 2H), 3.39 (s, 3H), 3.29-3.19 (m, 2H), 2.97-2.85 (m, 1H), 2.75 (s, 3H), 2.72-2.59 (m, 2H), 2.21-1.98 (m, 3H), 1.42 (s, 9H); LC-MS (ESI+) m/z 534.3 (M+H)⁺.

### Step 2—3-(3-methyl-5-{4-[(3R)-3-(methylamino)pyrrolidin-1-yl]phenyl}-2-oxo-1,3-benzodiazol-1-yl)piperidine-2,6-dione Trifluoroacetate

To a solution of tert-butyl N-[(3R)-1-{4-[1-(2,6-dioxopiperidin-3-yl)-3-methyl-2-oxo-1,3-benzodiazol-5-yl]phenyl}pyrrolidin-3-yl]-N-methylcarbamate (50 mg, 0.09 mmol) in DCM (2.5 mL) was added TFA (0.5 mL) dropwise at rt. The reaction mixture was stirred for 1 h at rt under nitrogen atmosphere. On completion, the reaction mixture was concentrated under reduced pressure. The residue was triturated with Et₂O to afford the title compound (45 mg) as a yellow solid. LC/MS (ESI, m/z): [(M+H)]⁺=434.2.

### Example 1.186. Preparation of Tert-butyl 4-(2-(3-bromophenoxy)ethyl)piperazine-1-carboxylate (Intermediate GE)

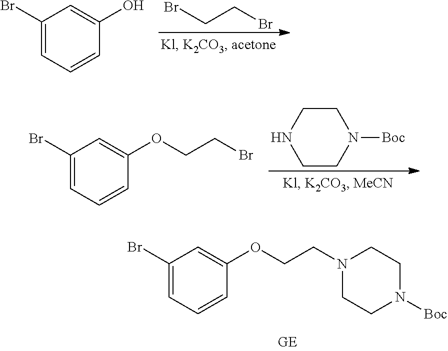

### Step-1—1-Bromo-3-(2-bromoethoxy)benzene

To a solution of 3-bromophenol (5 g, 28.9 mmol) and 1, 2-dibromoethane (32.5 g, 173 mmol) in acetone (50 mL) was added potassium carbonate (11.9 g, 86.7 mmol) and potassium iodide (71.9 mg, 433 μmol). The mixture was stirred at 60° C. for 12 hours. On completion, the reaction mixture was partitioned between water (100 mL) and ethyl acetate (400 mL). The organic phase was separated, dried over with anhydrous sodium sulfate, filtered and concentrated under reduced pressure to give a residue. The residue was purified by column chromatography (SiO₂, Petroleum ether/Ethyl acetate=20/1 to 1/1) to give the title compound (4.2 g, 51% yield) as a yellow oil. ¹H NMR (400 MHz, CHLOROFORM-d) δ=7.22-7.07 (m, 3H), 6.94-6.80 (m, 1H), 4.39-4.21 (m, 2H), 3.65 (t, J=6.4 Hz, 2H).

### Step 2—tert-butyl 4-(2-(3-bromophenoxy)ethyl)piperazine-1-carboxylate

To a solution of 1-bromo-3-(2-bromoethoxy) benzene (4.1 g, 14.6 mmol) and tert-butyl piperazine-1-carboxylate (2.73 g, 14.6 mmol, CAS #57260-71-6) in acetonitrile (60 mL) was added potassium carbonate (2.43 g, 17.5 mmol) and potassium iodide (2.43 g, 14.6 mmol). The mixture was stirred at 40° C. for 12 hours. On completion, the reaction mixture was filtered and concentrated under reduced pressure to give a residue. The residue was purified by column chromatography (SiO₂, Petroleum ether/Ethyl acetate=40/1 to 1/1) to give the title compound (4.2 g, 74% yield) as a yellow gum. ¹H NMR (400 MHz, CHLOROFORM-d) δ=7.19-7.06 (m, 3H), 6.88-6.78 (m, 1H), 4.11 (t, J=5.6 Hz, 2H), 3.59-3.42 (m, 4H), 2.84 (t, J=5.6 Hz, 2H), 2.63-2.50 (m, 4H), 1.48 (s, 9H).

### Example 1.187. Preparation of 3-(3-methyl-2-oxo-5-{3-[2-(piperazin-1-yl)ethoxy]phenyl}-1,3-benzodiazol-1-yl)piperidine-2,6-dione (Intermediate GF)

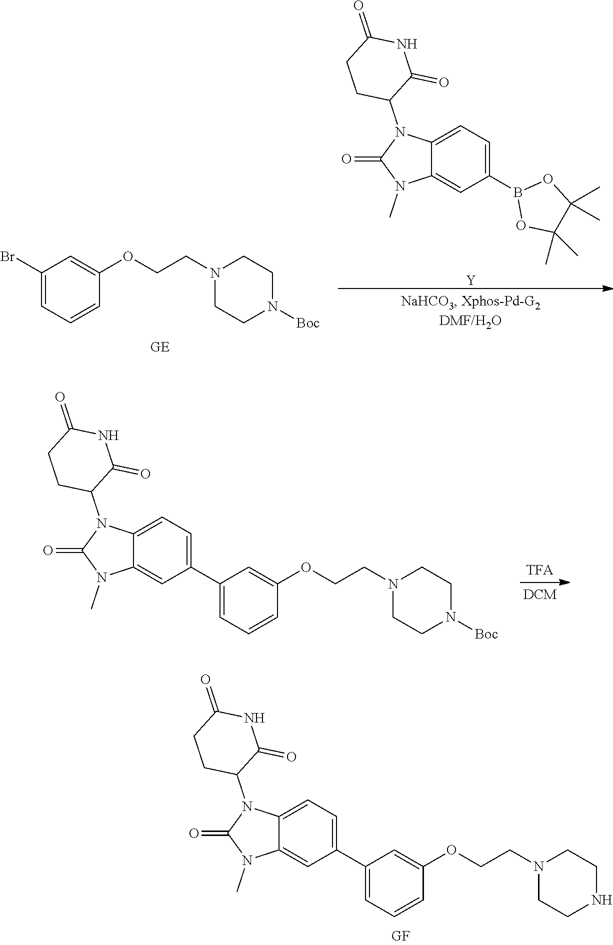

### Step 1—Tert-butyl 4-(2-(3-(1-(2,6-dioxopiperidin-3-yl)-3-methyl-2-oxo-2,3-dihydro-1H-benzo[d]imidazol-5-yl)phenoxy)ethyl)piperazine-1-carboxylate

To a solution of tert-butyl 4-[2-(3-bromophenoxy) ethyl]piperazine-1-carboxylate (4 g, 10.3 mmol, Intermediate GE) and 3-[3-methyl-2-oxo-5-(4,4,5,5-tetramethyl-1,3,2-dioxaborolan-2-yl)benzimidazol-1-yl]piperidine-2,6-dione (3.08 g, 7.99 mmol, Intermediate Y) in water (5 mL) and dimethylformamide (50 mL) was added sodium bicarbonate (1.34 g, 15.9 mmol) and chloro (2-dicyclohexylphosphino-2′,4′,6′-triisopropyl-1,1′-biphenyl)[2-(2′-amino-1,1′-biphenyl)]palladium(II) (628 mg, 798 μmol). The mixture was stirred at 80° C. for 4 hours. On completion, the reaction mixture was partitioned between ethyl acetate (300 mL) and water (100 mL). The organic phase was separated, washed with brine (100 mL×2), dried over with anhydrous sodium sulfate, filtered and concentrated under reduced pressure to give a residue. The crude product was purified by reversed-phase HPLC (0.1% FA condition) to give the title compound (1.55 g, 33% yield) as an off-white solid. ¹H NMR (400 MHz, DMSO-d₆) δ=11.19 (s, 1H), 7.57 (d, J=1.6 Hz, 1H), 7.46-7.38 (m, 2H), 7.35-7.28 (m, 2H), 7.27-7.20 (m, 1H), 6.98 (dd, J=2.0, 8.1 Hz, 1H), 5.46 (dd, J=5.6, 12.8 Hz, 1H), 4.23 (t, J=5.6 Hz, 2H), 3.48 (s, 3H), 3.39 (s, 4H), 3.03-2.94 (m, 1H), 2.83-2.69 (m, 4H), 2.54-2.49 (m, 4H), 2.17-2.06 (m, 1H), 1.45 (s, 9H); LC-MS (ESI+) m/z 564.1 (M+H)⁺.

### Step 2—3-(3-Methyl-2-oxo-5-{3-[2-(piperazin-1-yl)ethoxy]phenyl}-1,3-benzodiazol-1-yl)piperidine-2,6-dione Trifluoroacetate

To a solution of tert-butyl 4-(2-{3-[1-(2,6-dioxopiperidin-3-yl)-3-methyl-2-oxo-1,3-benzodiazol-5-yl]phenoxy}ethyl)piperazine-1-carboxylate (50 mg, 0.09 mmol) in DCM (2.5 mL) was added TFA (0.5 mL) dropwise at rt. The reaction mixture was stirred for 1 h at rt under nitrogen atmosphere. On completion, the reaction mixture was concentrated under reduced pressure. The residue was triturated with Et₂O to afford the title compound (48 mg) as a yellow solid. LC/MS (ESI, m/z): [(M+H)]⁺=464.2.

### Example 1.188. Preparation of Tert-butyl 7-(5-{2-[1-(2,6-dioxopiperidin-3-yl)-3-methyl-2-oxo-1,3-benzodiazol-5-yl]ethynyl}pyrimidin-2-yl)-6-oxo-2,7-diazaspiro[4.4]nonane-2-carboxylate (Intermediate GG)

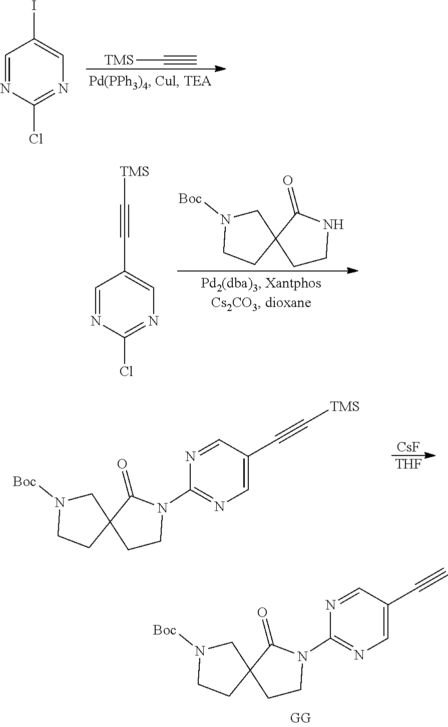

### Step 1—2-Chloro-5-((trimethylsilyl)ethynyl)pyrimidine

A mixture of 2-chloro-5-iodopyrimidine (10 g, 41.59 mmol, CAS #32779-38-7), ethynyltrimethylsilane (4.29 g, 43.67 mmol, 6.05 mL), CuI (237.64 mg, 1.25 mmol), TEA (8.42 g, 83.18 mmol, 11.58 mL) and Pd(PPh₃)₂Cl₂ (875.81 mg, 1.25 mmol) in THF (100 mL) was degassed and purged with N₂ three times. Then the mixture was stirred at 50° C. for 16 hr under N₂ atmosphere. On completion, the reaction mixture was filtered and the cake was wash with EA (150 mL). The combined filtrate was concentrated under reduced pressure to give a residue. The residue was purified by column chromatography (SiO₂, Petroleum ether/Ethyl acetate=100/1 to 10/1) to give the title compound (7 g, 80% yield) as yellow solid. ¹H NMR (400 MHz, CDCl₃-d) δ ppm 8.67 (s, 2H) 0.29 (s, 9H).

### Step 2—Tert-butyl 6-oxo-7-(5-((trimethylsilyl)ethynyl)pyrimidin-2-yl)-2,7-diazaspiro[4.4]nonane-2-carboxylate

A mixture of 2-chloro-5-((trimethylsilyl) ethynyl) pyrimidine (2.81 g, 13.33 mmol, 1.00 eq), tert-butyl 6-oxo-2, 7-diazaspiro[4.4]nonane-2-carboxylate (3.2 g, 13.32 mmol, CAS #1194376-44-7), Pd₂ (dba)₃ (1.22 g, 1.33 mmol), Xantphos (770.53 mg, 1.33 mmol) and Cs₂CO₃ (13.02 g, 39.95 mmol) in dioxane (40 mL) was degassed and purged with N₂ three times. Then the mixture was stirred at 80° C. for 2.5 hrs under N₂ atmosphere. On completion, the reaction mixture was filtered and the cake was washed with EA (200 mL). The combined filtrate was concentrated to give a residue. The residue was purified by column chromatography (SiO₂, Petroleum ether/Ethyl acetate=100/1 to 3/1, TLC (PE/EA=3:1, Rf=0.4, uv 254 nm) to give the title compound (5.5 g, quant. yield) as gray solid. ¹H NMR (400 MHz, CDCl₃-d) δ=8.72 (s, 2H), 4.11-3.98 (m, 2H), 3.84-3.60 (m, 2H), 3.55-3.31 (m, 2H), 2.47-2.26 (m, 1H), 2.24-2.08 (m, 2H), 1.97-1.84 (m, 1H), 1.48 (s, 9H), 0.29 (s, 9H).

### Step 3—Tert-butyl 7-(5-ethynylpyrimidin-2-yl)-6-oxo-2,7-diazaspiro[4.4]nonane-2-carboxylate

To a solution of tert-butyl 1-oxo-2-[5-(2-trimethylsilylethynyl) pyrimidin-2-yl]-2, 7-diazaspiro[4.4]nonane-7-carboxylate (3.5 g, 8.4 mmol) in THF (35 mL) was added CsF (6.41 g, 42.21 mmol, 1.56 mL). The mixture was stirred at 25° C. for 16 hr. On completion, the reaction was filtered and the cake was washed with EA (200 mL). The combined filtrate was concentrated to give a residue. The residue was purified by column chromatography (SiO₂, Petroleum ether/Ethyl acetate=100/1 to 1/1) to give the title compound (2.58 g, 55% yield) as yellow solid. ¹H NMR (400 MHz, CDCl₃-d) δ ppm 8.75 (s, 2H) 4.04 (t, J=7.2 Hz, 2H) 3.68 (d, J=10.8 Hz, 2H) 3.36-3.56 (m, 2H) 3.34 (s, 1H) 2.27-2.47 (m, 1H) 2.06-2.24 (m, 2H) 1.85-1.95 (m, 1H) 1.47 (s, 9H).

### Example 1.189. Preparation of 3-{3-methyl-2-oxo-5-[2-(2-{1-oxo-2,7-diazaspiro[4.4]nonan-2-yl}pyrimidin-5-yl)ethynyl]-1,3-benzodiazol-1-yl}piperidine-2,6-dione (Intermediate GH)

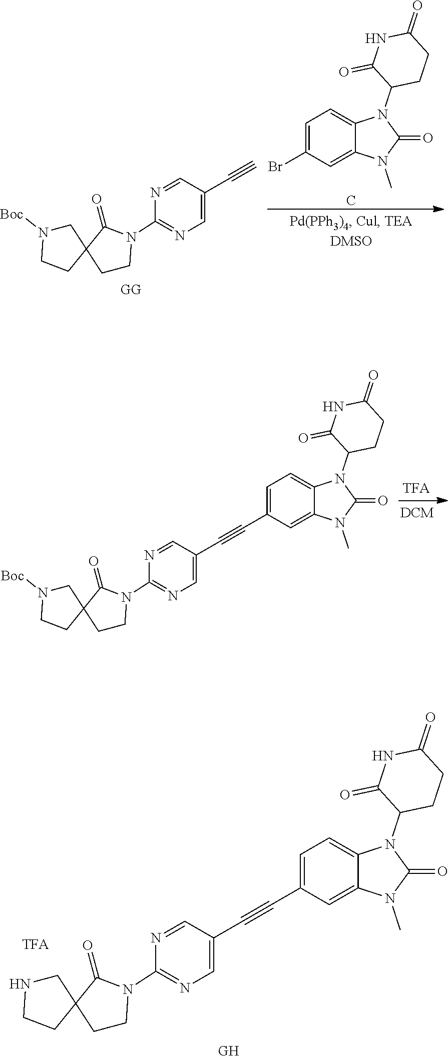

### Step 1—Tert-butyl 7-(5-((1-(2,6-dioxopiperidin-3-yl)-3-methyl-2-oxo-2,3-dihydro-1H-benzo[d]imidazol-5-yl)ethynyl)pyrimidin-2-yl)-6-oxo-2,7-diazaspiro[4.4]nonane-2-carboxylate

To a solution of 3-(5-bromo-3-methyl-2-oxo-2, 3-dihydro-1H-benzo[d]imidazol-1-yl) piperidine-2,6-dione (1.76 g, 5.20 mmol, Intermediate C) in DMSO (27 mL) was added CuI (99.01 mg, 519.87 μmol), Pd(PPh₃)₄ (600.74 mg, 519.87 μmol) and TEA (1.58 g, 15.60 mmol, 2.17 mL). The mixture was stirred at 25° C. for 0.5 hrs under N₂. Then tert-butyl 2-(5-ethynylpyrimidin-2-yl)-1-oxo-2,7-diazaspiro[4.4]nonane-7-carboxylate (1.78 g, 5.20 mmol, Intermediate GG) was added, and the mixture was stirred at 90° C. for 2.5 hrs. On completion, the reaction mixture was diluted with H₂O (150 mL) and extracted with EA (100 mL×4). The combined organic layers were washed with brine (150 mL), dried over Na₂SO₄, filtered and concentrated under reduced pressure to give a residue. The residue was triturated with EA (50 mL) at 25° C. for 3 mins. Then the mixture was filtered and the filter cake was dried under vacuum to give the title compound (1.65 g, 51% yield) as yellow solid. ¹HNMR (400 MHz, DMSO-d6) δ ppm 11.14 (s, 1H) 8.91 (s, 2H) 7.46 (s, 1H) 7.32 (d, J=8.0 Hz, 1H) 7.22 (d, J=8.2 Hz, 1H) 5.42 (dd, J=12.8, 5.2 Hz, 1H) 3.85-4.13 (m, 2H) 3.42-3.55 (m, 2H) 3.36-3.40 (m, 3H) 3.29-3.33 (m, 2H) 2.83-2.96 (m, 1H) 2.59-2.78 (m, 2H) 2.02-2.18 (m, 4H) 1.90-2.01 (m, 1H) 1.41 (s, 9H).

### Step 2—3-{3-methyl-2-oxo-5-[2-(2-{1-oxo-2,7-diazaspiro[4.4]nonan-2-yl}pyrimidin-5-yl)ethynyl]-1,3-benzodiazol-1-yl}piperidine-2,6-dione Trifluoroacetate

To a solution of tert-butyl 7-(5-{2-[1-(2,6-dioxopiperidin-3-yl)-3-methyl-2-oxo-1,3-benzodiazol-5-yl]ethynyl}pyrimidin-2-yl)-6-oxo-2,7-diazaspiro[4.4]nonane-2-carboxylate (55 mg, 0.092 mmol) in DCM (2.5 mL) was added TFA (0.5 mL) dropwise at rt. The reaction mixture was stirred for 1 h at rt under nitrogen atmosphere. On completion, the reaction mixture was concentrated under reduced pressure. The residue was triturated with Et₂O to afford the title compound (50 mg) as a yellow solid. LC/MS (ESI, m/z): [(M+H)]⁺=500.3.

### Example 1.190. Preparation of Tert-butyl 8-(but-3-yn-1-yl)-5-oxa-2,8-diazaspiro[3.5]nonane-2-carboxylate (Intermediate GI)

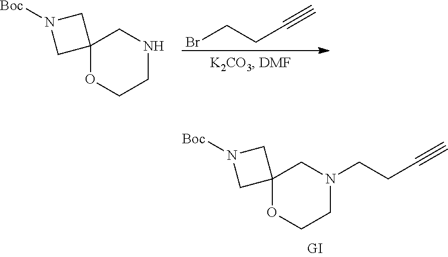

To a solution of tert-butyl 5-oxa-2, 8-diazaspiro[3.5]nonane-2-carboxylate (500 mg, 2.19 mmol, CAS #1251011-05-8) in DMF (8 mL) was added K₂CO₃ (605.40 mg, 4.38 mmol) at 0° C. and stirred for 10 min. Then 4-bromobut-1-yne (349.52 mg, 2.63 mmol, CAS #38771-21-0) in DMF (4 mL) was added to the mixture at 0° C. and then the mixture was stirred at 25° C. for 12 hr. On completion, the reaction mixture was filtered and the filtrate was concentrated in vacuo. The residue was purified by column chromatography (SiO₂, PE:EA=10:1 to 3:1, P:Rf=0.3 (PE:EA=3:1) to give the title compound (1.5 g, 24% yield) as colorless oil. ¹H NMR (400 MHz, CDCl₃-d) δ=3.88-3.77 (m, 4H), 3.70 (s, 2H), 2.59 (s, 4H), 2.47 (s, 2H), 2.40 (s, 2H), 2.01 (s, 1H), 1.46 (s, 9H).

### Example 1.191. Preparation of 3-[2-hydroxy-3-methyl-5-(4-{5-oxa-2,8-diazaspiro[3.5]nonan-8-yl}butyl)-hexahydro-2H-1,3-benzodiazol-1-yl]piperidine-2,6-diol (Intermediate GJ)

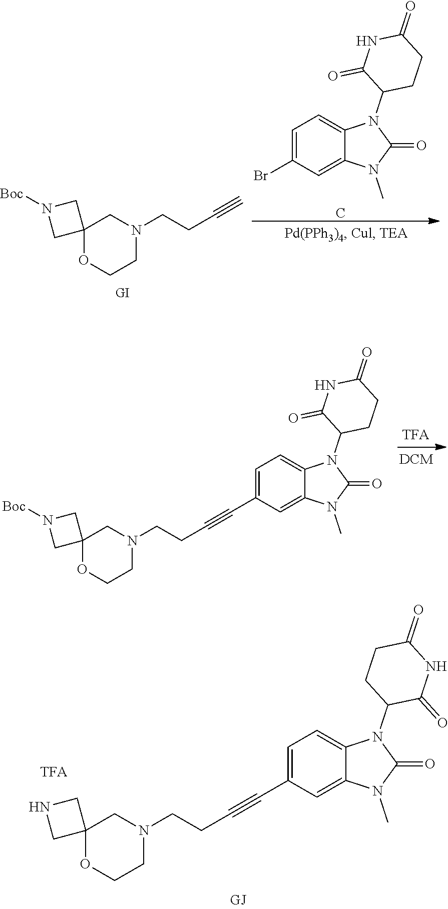

### Step 1—Tert-butyl 8-(4-(1-(2,6-dioxopiperidin-3-yl)-3-methyl-2-oxo-2,3-dihydro-1H-benzo[d]imidazol-5-yl)but-3-yn-1-yl)-5-oxa-2,8-diazaspiro[3.5]nonane-2-carboxylate

To a solution of 3-(5-bromo-3-methyl-2-oxo-benzimidazol-1-yl) piperidine-2, 6-dione (1.3 g, 3.84 mmol, Intermediate C), tert-butyl 8-but-3-ynyl-5-oxa-2, 8-diazaspiro[3.5]nonane-2-carboxylate (1.08 g, 3.84 mmol, Intermediate GI), Pd(PPh₃)₄ (444.24 mg, 384.44 μmol) and CuI (73.22 mg, 384.4 μmol) in DMSO (20 mL) was added TEA (1.95 g, 19.22 mmol, 2.68 mL). The mixture was stirred at 80° C. for 12 hr. On completion, H₂O (20 mL) was added to the reaction, then extracted with EA (20 mL×3). The organic layer was dried over Na₂SO₄, filtered and concentrated. The residue was purified by column chromatography (SiO₂, PE:EA=1:1 to 0:1, P: Rf=0.5 (PE:EA=0:1)) to give the product (2 g, 48% purity). The crude product was purified by reversed-phase HPLC (0.1% FA) to give the title compound (568.78 mg, 27% yield) as yellow gum. ¹H NMR (400 MHz, DMSO-d₆) δ=11.10 (s, 1H), 9.66 (s, 1H), 7.24 (s, 1H), 7.10 (s, 2H), 5.48-5.28 (m, 1H), 4.20 (t, J=6.4 Hz, 2H), 3.76-3.64 (m, 4H), 3.62-3.47 (m, 4H), 3.33 (s, 3H), 2.96-2.83 (m, 1H), 2.78 (t, J=6.4 Hz, 2H), 2.73-2.59 (m, 2H), 2.02 (m, 1H), 1.83 (s, 2H), 1.39-1.31 (m, 9H).

### Step 2—3-[2-hydroxy-3-methyl-5-(4-{5-oxa-2,8-diazaspiro[3.5]nonan-8-yl}butyl)-hexahydro-2H-1,3-benzodiazol-1-yl]piperidine-2,6-diol Trifluoroacetate

To a solution of tert-butyl 8-{4-[1-(2,6-dioxopiperidin-3-yl)-3-methyl-2-oxo-1,3-benzodiazol-5-yl]but-3-yn-1-yl}-5-oxa-2,8-diazaspiro[3.5]nonane-2-carboxylate (50 mg, 0.09 mmol) in DCM (2.5 mL) was added TFA (0.5 mL) dropwise at rt. The reaction mixture was stirred for 1 h at rt under nitrogen atmosphere. On completion, the reaction mixture was concentrated under reduced pressure. The residue was triturated with Et₂O to afford the title compound (45 mg) as a yellow solid. LC/MS (ESI, m/z): [(M+H)]⁺=438.2.

### Example 1.192. Preparation of 3-[3-Methyl-5-[[4-(methylaminomethyl)-1-piperidyl]methyl]-2-oxo-benzimidazol-1-yl]piperidine-2,6-dione (Intermediate GK)

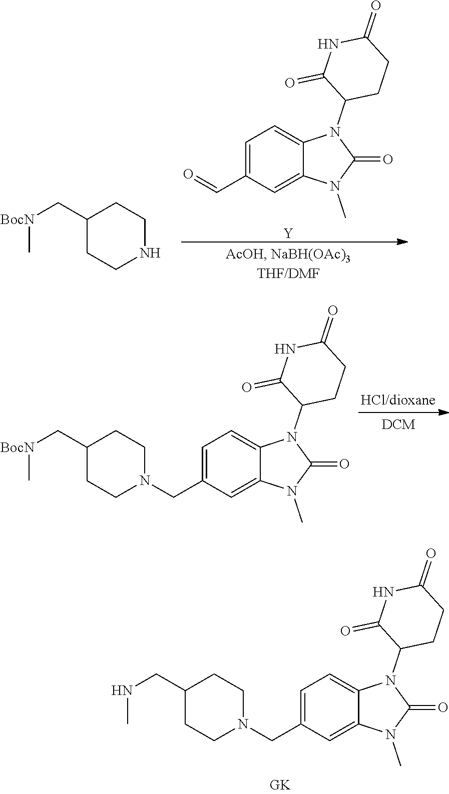

### Step 1—Tert-butyl N-[[1-[[1-(2,6-dioxo-3-piperidyl)-3-methyl-2-oxo-benzimidazol-5-yl]methyl]-4-piperidyl]methyl]-N-methyl-carbamate

To a solution of tert-butyl N-methyl-N-(4-piperidylmethyl)carbamate (1.91 g, 8.35 mmol, CAS #138022-04-5) and 1-(2,6-dioxo-3-piperidyl)-3-methyl-2-oxo-benzimidazole-5-carbaldehyde (2 g, 6.96 mmol, Intermediate Y) in tetrahydrofuran (20 mL) and dimethylformamide (20 mL) was added acetic acid (836 mg, 13.9 mmol) at 25° C. and it was stirred 0.5 hour. Then to the mixture was added sodium triacethoxy borohydride (4.43 g, 20.9 mmol) and it was stirred at 25° C. for 12 hours. On completion, the reaction mixture was quenched with 1 N sodium bicarbonate solution until the pH=8 at 25° C., diluted with water (80 mL), and extracted with ethyl acetate (80 mL×3). The combined organic layers were washed with brine (80 mL×3), dried over anhydrous sodium sulfate, filtered and concentrated under reduced pressure to give the title compound (3.48 g) as a yellow gum. LC-MS (ESI+) m/z 500.3 (M+H)⁺.

### Step 2—3-[3-Methyl-5-[[4-(methylaminomethyl)-1-piperidyl]methyl]-2-oxo-benzimidazol-1-yl]piperidine-2,6-dione

To a solution of tert-butyl N-[[1-[[1-(2,6-dioxo-3-piperidyl)-3-methyl-2-oxo-benzimidazol-5-yl]methyl]-4-piperidyl]methyl]-N-methyl-carbamate (3.28 g, 6.57 mmol) in dichloromethane (20 mL) was added HCl/dioxane (4 M, 1.64 mL). The mixture was stirred at 25° C. for 0.5 hour. On completion, the reaction mixture was concentrated under reduced pressure to give a residue to give the title compound (2.86 g, HCl salt) as a yellow gum. LC-MS (ESI+) m/z 400.2 (M+H)⁺.

### Example 1.193. Preparation of (R)-tert-butyl 4-(methyl((1-(4-(4,4,5,5-tetramethyl-1,3,2-dioxaborolan-2-yl)phenyl)pyrrolidin-3-yl)methyl)amino)piperidine-1-carboxylate (Intermediate GL)

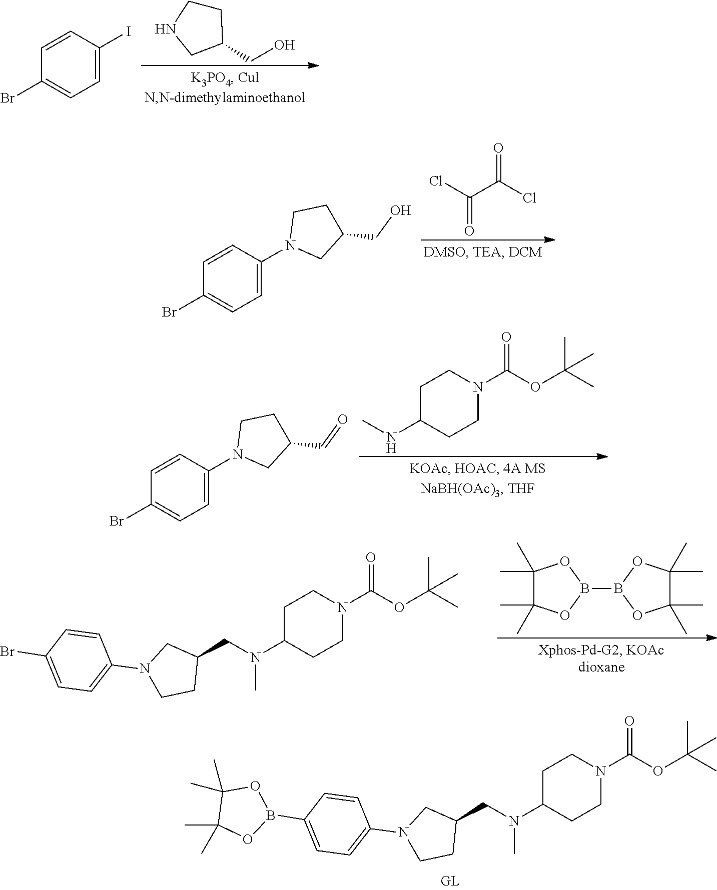

### Step 1—(S)-(1-(4-bromophenyl)pyrrolidin-3-yl)methanol

To a solution of (S)-pyrrolidin-3-ylmethanol (7.00 g, 69.2 mmol) and 1-bromo-4-iodo-benzene (9.79 g, 34.6 mmol) in N,N-dimethylaminoethanol (70 mL) was added K₃PO₄·H₂O (15.9 g, 69.2 mmol) and CuI (1.32 g, 6.92 mmol). The mixture was stirred at 55° C. for 84 h under N₂. On completion, the reaction mixture was quenched with H₂O (50 mL), and then extracted with EtOAc (50 mL×3). The combined organic layers were washed with brine (50 mL×3), dried over Na₂SO₄, filtered and concentrated under reduced pressure to give a residue. The residue was purified by flash silica gel chromatography (ISCO®; 20 g SepaFlash® Silica Flash Column, Eluent of 0˜35% Ethylacetate/Petroleum ether gradient @ 100 mL/min) to give the title compound (4.20 g, 46% yield) as a white solid. ¹H NMR (400 MHz, DMSO-d₆) δ=7.32-7.21 (m, 2H), 6.49-6.42 (m, 2H), 4.70 (t, J=5.3 Hz, 1H), 3.47-3.37 (m, 2H), 3.29-3.16 (m, 2H), 3.23-3.14 (m, 1H), 2.98 (dd, J=6.4, 9.6 Hz, 1H), 2.42 (td, J=6.8, 13.9 Hz, 1H), 2.08-2.00 (m, 1H), 1.74 (qd, J=7.6, 12.4 Hz, 1H).

### Step 2—Methyl (S)-1-(4-bromophenyl)pyrrolidine-3-carbaldehyde

To a solution of DMSO (1.30 g, 16.6 mmol) in DCM (68 mL) was added oxalyl dichloride (1.01 g, 7.96 mmol) at −70° C. under N₂. The mixture was stirred for 15 min. Then, a solution of (S)-(1-(4-bromophenyl)pyrrolidin-3-yl)methanol (1.70 g, 6.64 mmol) in DCM (17 mL) was added at −70° C. and stirred for 30 min. Then, TEA (3.36 g, 33.2 mmol) was added to the reaction at −70° C. and it was stirred at −70° C. for 1 h. On completion, the reaction mixture was quenched with water (100 mL), and extracted with DCM (100 mL×3). The combined organic layers were washed with brine (80 mL×3), dried over Na₂SO₄ and evaporated to give the title compound (1.7 g) as a white solid. ¹H NMR (400 MHz, DMSO-d₆) δ=9.66 (d, J=1.2 Hz, 1H), 7.31-7.27 (m, 2H), 6.53-6.50 (m, 2H), 3.55 (dd, J=4.0, 9.6 Hz, 1H), 3.36-3.29 (m, 2H), 3.20-3.15 (m, 2H), 2.28-2.23 (m, 1H), 2.21-2.14 (m, 1H).

### Step 3—(R)-tert-butyl 4-(((1-(4-bromophenyl)pyrrolidin-3-yl)methyl)(methyl)amino)piperidine-1-carboxylate

To a solution of tert-butyl 4-(methylamino)piperidine-1-carboxylate (1.15 g, 5.35 mmol, CAS #147539-41-1) and (S)-1-(4-bromophenyl)pyrrolidine-3-carbaldehyde (1.70 g, 5.35 mmol) in THF (17 mL) was added KOAc (1.58 g, 16.1 mmol), HOAc (964 mg, 16.1 mmol) and 4 Å molecular sieves (1.7 g, 5.35 mmol). The mixture was stirred at rt for 30 min. Then NaBH(OAc)₃ (3.40 g, 16.1 mmol) was added and stirred at 25° C. for 2 h. On completion, the reaction mixture was quenched with water (20 mL), and extracted with EtOAc (20 mL×3). The combined organic layers were washed with brine (10 mL×3), dried over Na₂SO₄ and evaporated to give the title compound (3.70 g) as an orange oil. ¹H NMR (400 MHz, DMSO-d₆) δ=7.35 (d, J=8.8 Hz, 2H), 6.53 (d, J=8.8 Hz, 2H), 4.12 (br d, J=3.2 Hz, 1H), 3.56 (br d, J=6.4 Hz, 1H), 3.36 (dt, J=3.2, 8.6 Hz, 4H), 3.26 (br d, J=8.4 Hz, 1H), 3.18-3.03 (m, 2H), 2.90-2.64 (m, 6H), 2.34-2.20 (m, 1H), 2.05-2.05 (m, 1H), 2.17-2.05 (m, 1H), 1.97 (s, 6H), 1.90-1.78 (m, 1H), 1.68-1.51 (m, 2H), 1.46 (s, 9H), 1.23 (t, J=7.1 Hz, 1H), 1.27-1.17 (m, 1H).

### Step 4—(R)-tert-butyl 4-(methyl((1-(4-(4,4,5,5-tetramethyl-1,3,2-dioxaborolan-2-yl)phenyl)pyrrolidin-3-yl)methyl)amino)piperidine-1-carboxylate

A solution of (R)-tert-butyl 4-(((1-(4-bromophenyl)pyrrolidin-3-yl)methyl)(methyl)amino)piperidine-1-carboxylate (2.00 g, 4.42 mmol), 4,4,5,5-tetramethyl-2-(4,4,5,5-tetramethyl-1,3,2-dioxaborolan-2-yl)-1,3,2-dioxaborolane (2.25 g, 8.84 mmol), XPHOS-PD-G2 (348 mg, 442 umol) and KOAc (868 mg, 8.84 mmol) in dioxane (40 mL) was degassed and purged with N₂ three times. Then the mixture was stirred at 90° C. for 12 h under N₂ atmosphere. On completion, the reaction mixture was quenched with water (40 mL), and extracted with EtOAc (40 mL×3). The combined organic layers were washed with brine (40 mL×3), dried over Na₂SO₄ and evaporated. The residue was purified by flash silica gel chromatography (ISCO®; 40 g SepaFlash® Silica Flash Column, Eluent of 91% Dichloromethane/Methanol ether gradient @80 mL/min) to give the title compound (1.10 g, 44% yield) as a red solid. ¹H NMR (400 MHz, DMSO-d₆) δ=7.47 (br d, J=8.0 Hz, 2H), 6.48 (br d, J=8.4 Hz, 2H), 4.02-3.89 (m, 2H), 3.33 (br s, 4H), 3.28-3.17 (m, 2H), 3.01 (br s, 2H), 2.69 (br d, J=9.2 Hz, 4H), 2.33-2.04 (m, 4H), 1.72 (br d, J=2.0 Hz, 2H), 1.39 (s, 9H), 1.29 (br s, 1H), 1.26 (s, 12H).

### Example 1.194. Preparation of 1-(2,6-dioxopiperidin-3-yl)-3-methyl-2-oxo-2,3-dihydro-1H-benzo[d]imidazole-4-carbaldehyde (Intermediate GO)

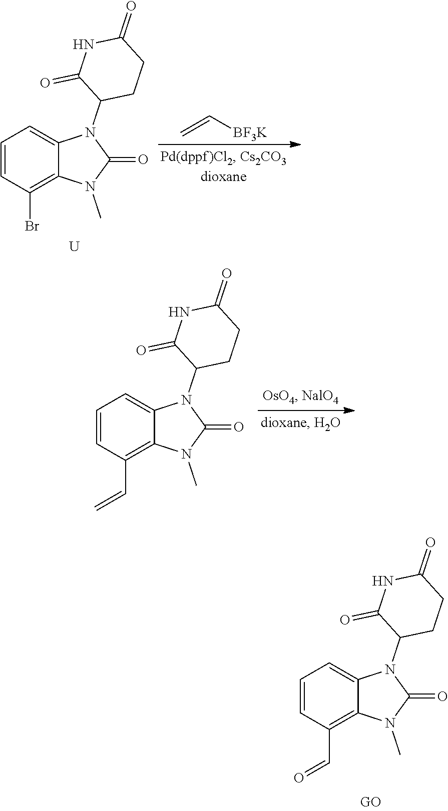

### Step 1—3-(3-Methyl-2-oxo-4-vinyl-2,3-dihydro-1H-benzo[d]imidazol-1-yl)piperidine-2,6-dione

To a solution of 3-(4-bromo-3-methyl-2-oxo-benzimidazol-1-yl)piperidine-2,6-dione (15 g, 44 mmol, Intermediate U) and potassium; trifluoro(vinyl)boranuide (17.83 g, 133.0 mmol, CAS #13682-77-4) in dioxane (300 mL) was added Cs₂CO₃ (2 M, 44.36 mL) and Pd(dppf)Cl₂·CH2Cl₂ (3.62 g, 4.44 mmol). The mixture was then stirred at 85° C. for 12 hr. On completion, the reaction was poured into water (100 mL) and extracted with EA (100 mL×3). The combined organic layers were washed with brine (100 mL), dried over Na₂SO₄ filtered and concentrated under reduced pressure to give a residue. The residue was triturated with PE:EA=1:1 (50 mL), filtered and dried in vacuo to the title compound (12 g, 95% yield) as yellow solid. LC-MS (ESI⁺) m/z 286.0 (M+H)⁺.

### Step 2—1-(2,6-Dioxopiperidin-3-yl)-3-methyl-2-oxo-2,3-dihydro-1H-benzo[d]imidazole-4-carbaldehyde

To a solution of 3-(3-methyl-2-oxo-4-vinyl-benzimidazol-1-yl)piperidine-2,6-dione (2.8 g, 9.8 mmol) in dioxane (50 mL) and H₂O (5 mL) was added NaIO₄ (8.40 g, 39.3 mmol, 2.18 mL), 2,6-dimethylpyridine (2.12 g, 19.6 mmol) and OsO₄ (499.02 mg, 1.96 mmol, 101.84 uL) at 0° C. The mixture was stirred at 0° C. for 1 hr. The reaction mixture was quenched with saturated Na₂S₂O₃ solution (100 mL) at 0° C., and then diluted with H₂O (100 mL), and extracted with ethyl acetate (200 mL×2). The combined organic layers were washed with brine (100 mL), dried over Na₂SO₄, filtered and concentrated under reduced pressure to give the title compound (700 mg, 25% yield) as black solid. LC-MS (ESI⁺) m/z 287.8 (M+H)⁺.

### Example 1.195. Preparation of 3-[3-methyl-5-(4-{methyl[(3S)-pyrrolidin-3-ylmethyl]amino}piperidin-1-yl)-2-oxo-1,3-benzodiazol-1-yl]piperidine-2,6-dione (Intermediate GP)

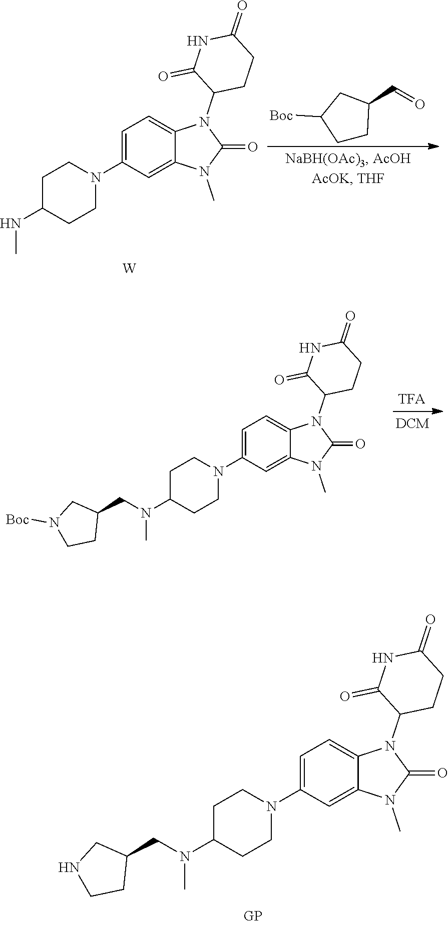

### Step 1—(3R)-tert-butyl 3-(((1-(1-(2,6-dioxopiperidin-3-yl)-3-methyl-2-oxo-2,3-dihydro-1H-benzo[d]imidazol-5-yl)piperidin-4-yl)(methyl)amino)methyl)pyrrolidine-1-carboxylate

To a solution of 3-(3-methyl-5-(4-(methylamino)piperidin-1-yl)-2-oxo-2,3-dihydro-1H-benzo[d]imidazol-1-yl)piperidine-2,6-dione (990 mg, 2.67 mmol, Intermediate W) in THF (15 mL) was added AcOH (480 mg, 8.00 mmol), KOAc (785 mg, 8.00 mmol), 4 Å molecular sieves (1 g, 2.67 mmol) and (S)-tert-butyl 3-formylpyrrolidine-1-carboxylate (637 mg, 3.20 mmol, CAS #191348-04-6), and it was stirred at 0° C. for 30 min. Then, NaBH(OAc)₃ (1.69 g, 8.00 mmol) was added into the mixture and it was stirred at 20° C. for 12 hr. On completion, the mixture was poured into NH₄Cl solution (30 ml), then extracted with DCM (10 ml×3) and the organic phase was concentrated. The residue was purified by column chromatography (SiO₂, Petroleum ether/Ethyl acetate=20/1-0/1, THF/Ethyl acetate=1:1) to give the title compound (930 mg, 63% yield). LC-MS (ESI+) m/z 555.5 (M+H)⁺. 1H NMR (400 MHz, DMSO-d6) δ=11.05 (s, 1H), 6.92 (d, J=8.4 Hz, 1H), 6.82 (d, J=2.0 Hz, 1H), 6.62 (dd, J=2.0, 8.6 Hz, 1H), 5.28 (dd, J=5.6, 12.9 Hz, 1H), 3.62 (br d, J=10.0 Hz, 2H), 3.30 (s, 5H), 3.20-3.12 (m, 1H), 2.99-2.83 (m, 2H), 2.67-2.57 (m, 4H), 2.37-2.29 (m, 4H), 2.21 (s, 3H), 2.01-1.95 (m, 1H), 1.89-1.82 (m, 1H), 1.78-1.69 (m, 2H), 1.59-1.50 (m, 3H), 1.39 (s, 9H).

### Step 2—3-[3-Methyl-5-(4-{methyl[(3S)-pyrrolidin-3-ylmethyl]amino}piperidin-1-yl)-2-oxo-1,3-benzodiazol-1-yl]piperidine-2,6-dione trifluoroacetate

To a stirred solution of tert-butyl (3R)-3-[({1-[1-(2,6-dioxopiperidin-3-yl)-3-methyl-2-oxo-1,3-benzodiazol-5-yl]piperidin-4-yl}(methyl)amino)methyl]pyrrolidine-1-carboxylate (45 mg, 0.081 mmol) in DCM (1 mL) was added TFA (0.2 mL) dropwise at rt. The resulting mixture was stirred for 1 h at rt under nitrogen atmosphere. On completion, the reaction mixture was concentrated under reduced pressure. The residue was purified by trituration with Et₂O to give the title compound (40.5 mg) as a white solid. LC/MS (ESI, m/z): [(M+1)]⁺=455.3.

### Example 1.196. Preparation of tert-butyl (1-((1-(2,6-dioxopiperidin-3-yl)-3-methyl-2-oxo-2,3-dihydro-1H-benzo[d]imidazol-5-yl)methyl)piperidin-4-yl)(methyl)carbamate (Intermediate GQ)

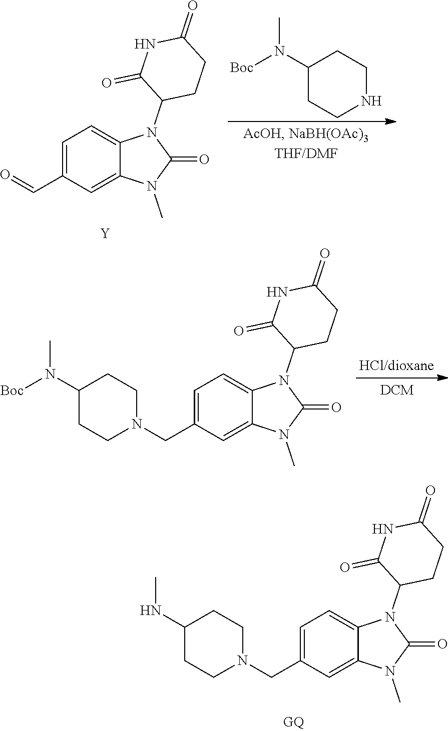

### Step 1—Tert-butyl methyl(piperidin-4-yl)carbamate

To a solution of tert-butyl N-methyl-N-(4-piperidyl) carbamate (1.79 g, 8.35 mmol, CAS #108612-54-0) and 1-(2, 6-dioxo-3-piperidyl)-3-methyl-2-oxo-benzimidazole-5-carbaldehyde (2 g, 6.96 mmol, Intermediate Y) in tetrahydrofuran (20 mL) and dimethylformamide (20 mL) was added acetic acid (836 mg, 13.9 mmol) at 25° C. for 0.5 hour, then to the mixture was added sodium triacethoxy borohydride (4.43 g, 20.9 mmol). The mixture was then stirred at 25° C. for 11.5 hour. On completion, the reaction mixture was quenched with 1 N sodium bicarbonate to pH=8 at 25° C., diluted with water (80 mL) and extracted with ethyl acetate (80 mL×3). The combined organic layers were washed with brine 240 mL (80 mL×3), dried over anhydrous sodium sulfate, filtered and concentrated under reduced pressure to give the title compound (3.38 g) as a yellow gum. LC-MS (ESI+) m/z 486.3 (M+H)⁺.

### Step 2—Tert-butyl (1-((1-(2,6-dioxopiperidin-3-yl)-3-methyl-2-oxo-2,3-dihydro-1H-benzo[d]imidazol-5-yl)methyl)piperidin-4-yl)(methyl)carbamate

To a solution of tert-butyl N-[1-[[1-(2,6-dioxo-3-piperidyl)-3-methyl-2-oxo-benzimidazol-5-yl]methyl]-4-piperidyl]-N-methyl-carbamate (3.18 g, 6.55 mmol) in dichloromethane (20 mL) was added hydrochloric acid/dioxane (4 M, 1.64 mL). The mixture was stirred at 25° C. for 0.5 hour. The reaction mixture was concentrated under reduced pressure to give the title compound (2.76 g, HCl) as a yellow gum. LC-MS (ESI+) m/z 386.2 (M+H)⁺.

### Example 1.197. Preparation of 3-[3-methyl-5-({4-[methyl({[(1r,4r)-4-aminocyclohexyl]methyl})amino]piperidin-1-yl}methyl)-2-oxo-1,3-benzodiazol-1-yl]piperidine-2,6-dione (Intermediate GR)

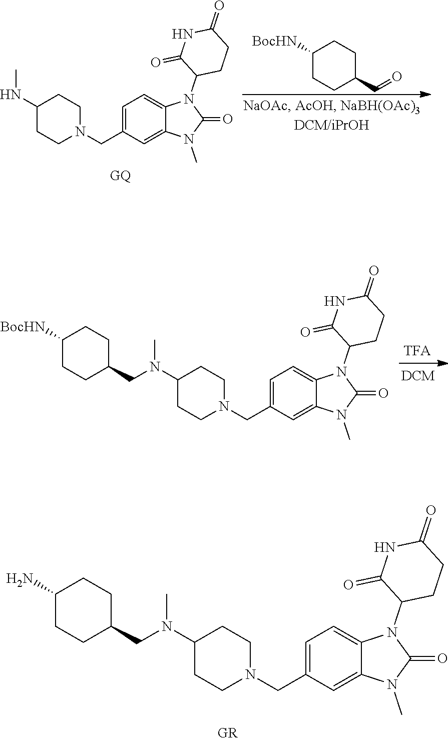 [SMILES: CNC1CCN(Cc2ccc3c(c2)n(C)c(=O)n3C2CCC(=O)NC2=O)CC1 ; CC(C)(C)OC(=O)N[C@H]1CC[C@H](C=O)CC1 ; CN(C[C@H]1CC[C@H](NC(=O)OC(C)(C)C)CC1)C1CCN(Cc2ccc3c(c2)n(C)c(=O)n3C2CCC(=O)NC2=O)CC1 ; CN(C[C@H]1CC[C@H](N)CC1)C1CCN(Cc2ccc3c(c2)n(C)c(=O)n3C2CCC(=O)NC2=O)CC1]

### Step 1—tert-butyl N-[4-[[[1-[[1-(2,6-dioxo-3-piperidyl)-3-methyl-2-oxo-benzimidazol-5-yl]methyl]-4-piperidyl]-methyl-amino]methyl]cyclohexyl]carbamate

To a solution of 3-[3-methyl-5-[[4-(methylamino)-1-piperidyl]methyl]-2-oxo-benzimidazol-1-yl]piperidine-2,6-dione (2.76 g, 6.54 mmol, HCl, Intermediate GQ) in dichloromethane (35 mL) and isopropyl alcohol (70 mL) was added sodium acetate (1.61 g, 19.6 mmol) at 25° C. for 30 minutes. Then to the mixture was added tert-butyl N-(4-formylcyclohexyl) carbamate (2.23 g, 9.81 mmol; CAS #181308-57-6) and acetic acid (786 mg, 13.1 mmol) at 25° C. and the mixture was stirred for 30 minutes. Next, to the mixture was added sodium triacethoxy borohydride (2.77 g, 13.1 mmol) at 0° C. Then the mixture was stirred at 25° C. for 11 hour. On completion, the reaction mixture was quenched with 1 N sodium bicarbonate until the pH=8 at 25° C., diluted with water (150 mL), and extracted with ethyl acetate (100 mL×3). The combined organic layers were washed with brine (100 mL×3), dried over anhydrous sodium sulfate, filtered and concentrated under reduced pressure to give a residue. The residue was purified by prep-HPLC (column: Phenomenex Synergi C18 150*25 mm*10 um; mobile phase: [water (FA)-ACN]; B %: 3%-22%, 10 min) freeze-drying to give the title compound (784 mg, 18% yield) as an off-white solid. ¹H NMR (400 MHz, DMSO-d₆) δ=11.11 (s, 1H), 7.12 (s, 1H), 7.08-7.04 (m, 1H), 7.00-6.96 (m, 1H), 6.69 (d, J=7.6 Hz, 1H), 5.39-5.34 (m, 1H), 3.55 (s, 2H), 3.34 (s, 3H), 3.20-3.07 (m, 1H), 2.95-2.86 (m, 3H), 2.78-2.67 (m, 1H), 2.66-2.59 (m, 1H), 2.49-2.42 (m, 1H), 2.36-2.27 (m, 2H), 2.25 (s, 3H), 2.06-1.98 (m, 3H), 1.83-1.63 (m, 6H), 1.54-1.46 (m, 2H), 1.37 (s, 10H), 1.18-1.03 (m, 2H), 0.93-0.76 (m, 2H); LC-MS (ESI+) m/z 597.2 (M+H)⁺.

### Step 2—3-[3-Methyl-5-({4-[methyl({[(1r,4r)-4-aminocyclohexyl]methyl})amino]piperidin-1-yl}methyl)-2-oxo-1,3-benzodiazol-1-yl]piperidine-2,6-dione Trifluoroacetate

To a stirred solution of tert-butyl N-[(1r,4r)-4-{[(1-{[1-(2,6-dioxopiperidin-3-yl)-3-methyl-2-oxo-1,3-benzodiazol-5-yl]methyl}piperidin-4-yl)(methyl)amino]methyl}cyclohexyl]carbamate (45 mg, 0.075 mmol) in DCM (1 mL) was added TFA (0.2 mL) dropwise at rt. The resulting mixture was stirred for 1 h at rt under nitrogen atmosphere. On completion, the reaction mixture was concentrated under reduced pressure. The residue was purified by trituration with Et₂O to give the title compound (50 mg) as a white solid. LC/MS (ESI, m/z): [(M+1)]⁺=497.4.

### Example 1.198. Preparation of Tert-butyl 4-[(3S)-pyrrolidin-3-yl]piperidine-1-carboxylate (Intermediate GS)

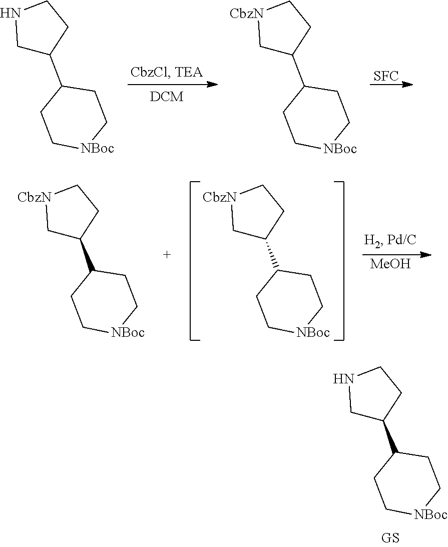

### Step 1—Tert-butyl 4-(1-benzyloxycarbonylpyrrolidin-3-yl)piperidine-1-carboxylate

To a solution of tert-butyl 4-pyrrolidin-3-ylpiperidine-1-carboxylate (4.9 g, 19 mmol, CAS #1314771-79-3) in dichloromethane (40 mL) was added triethylamine (7.80 g, 77 mmol) and benzyl carbonochloridate (6.57 g, 38.5 mmol). The mixture was stirred at 25° C. for 12 hrs. On completion, the reaction mixture was diluted with water (100 mL), and extracted with dichloromethane (200 mL×3). The organic layer was dried over anhydrous sodium sulfate, and concentrated under reduced pressure to give a residue. The residue was purified by silica gel column chromatography (Petroleum ether/Ethyl acetate=2:1) to give a white solid. The solid was purified by SFC (column: daicel chiralcel OJ (250 mm*50 mm, 10 um); mobile phase: [0.1% NH₃H₂O in MEOH]; B %: 20%-20%, 3.7; 80 min) to give tert-butyl 4-[(3S)-1-benzyloxycarbonylpyrrolidin-3-yl]piperidine-1-carboxylate (1.8 g, 25% yield) as white solid and tert-butyl 4-[(3R)-1-benzyloxycarbonylpyrrolidin-3-yl]piperidine-1-carboxylate (1.5 g, 20% yield) as white solid. LC-MS (ESI+) m/z 411.0 (M+Na)⁺.

### Step 2—Tert-butyl 4-[(3S)-pyrrolidin-3-yl]piperidine-1-carboxylate

To a suspension of Pd/C (0.8 g, 3.86 mmol, 10 wt %) in methanol (12 mL) was added a solution of tert-butyl 4-[(3S)-1-benzyloxycarbonylpyrrolidin-3-yl]piperidine-1-carboxylate (1.5 g, 3.9 mmol) in methanol (12 mL) under nitrogen. Then the mixture was degassed and purged with hydrogen three time, and the mixture was stirred at 25° C. for 12 hours under hydrogen (15 psi). On completion, the reaction mixture was filtered and the filtrate was concentrated under reduced pressure to give the title compound (800 mg) as a white solid. LC-MS (ESI+) m/z 255.5 (M+H)⁺.

### Example 1.199. Preparation of 3-(3-Methyl-5-{4-[4-(methylamino)cyclohexyl]piperazin-1-yl}-2-oxo-1,3-benzodiazol-1-yl)piperidine-2,6-dione (Intermediate GT)

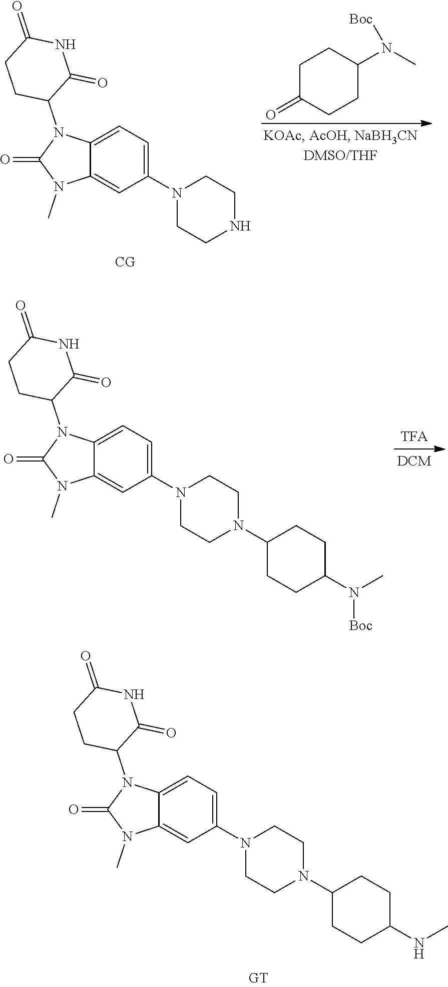 [SMILES: Cn1c(=O)n(C2CCC(=O)NC2=O)c2ccc(N3CCNCC3)cc21 ; CN(C(=O)OC(C)(C)C)C1CCC(=O)CC1 ; CN(C(=O)OC(C)(C)C)C1CCC(N2CCN(c3ccc4c(c3)n(C)c(=O)n4C3CCC(=O)NC3=O)CC2)CC1 ; CNC1CCC(N2CCN(c3ccc4c(c3)n(C)c(=O)n4C3CCC(=O)NC3=O)CC2)CC1]

### Step 1—tert-butyl N-[4-[4-[1-(2,6-dioxo-3-piperidyl)-3-methyl-2-oxo-benzimidazol-5-yl]piperazin-1-yl]cyclohexyl]-N-methyl-carbamate

To a solution of 3-(3-methyl-2-oxo-5-piperazin-1-yl-benzimidazol-1-yl)piperidine-2,6-dione (1.2 g, 3.49 mmol, Intermediate CG) in dimethyl sulfoxide (30 mL) and tetrahydrofuran (30 mL) was added potassium acetate (685 mg, 6.99 mmol). After stirring at 25° C. for 3 hours, acetic acid (629 mg, 10.4 mmol) and tert-butyl N-methyl-N-(4-oxocyclohexyl)carbamate (873 mg, 3.84 mmol; CAS #400899-84-5) were added. After the mixture was stirred at 25° C. for 12 hours, sodium cyanoborohydride (658 mg, 10.4 mmol) was added to the reaction mixture and the mixture was stirred at 25° C. for 9 hours. On completion, the reaction mixture was diluted with water (50 mL), extracted with ethyl acetate (100 mL×3), dried over anhydrous sodium sulfate, and concentrated under reduced pressure to give a residue. The residue was purified by perp-HPLC (column: Phenomenex Luna C18; 150*40 mm*15 um; mobile phase: [water (TFA)-ACN]; B %: 25%-35%, 10 min) to give the title compound (1.18 g, 60% yield) as a white solid. ¹H NMR (400 MHz, DMSO-d₆) δ=11.08 (s, 1H), 7.01 (d, J=8.8 Hz, 1H), 6.96-6.90 (m, 1H), 6.71 (m, 1H), 5.34-5.29 (m, 1H), 3.91 (d, J=4.0 Hz, 1H), 3.74 (s, 2H), 3.33 (s, 3H), 3.27-3.09 (m, 4H), 3.01-2.85 (m, 2H), 2.80-2.69 (m, 3H), 2.67 (s, 1H), 2.65-2.58 (m, 2H), 2.29-2.13 (m, 2H), 2.07-1.90 (m, 2H), 1.78 (d, J=9.2 Hz, 4H), 1.47 (s, 2H), 1.40 (s, 9H); LC-MS (ESI+) m/z 555.4 (M+H)⁺.

### Step 2—3-(3-Methyl-5-{4-[4-(methylamino)cyclohexyl]piperazin-1-yl}-2-oxo-1,3-benzodiazol-1-yl)piperidine-2,6-dione Trifluoroacetate

To a stirred solution of tert-butyl N-(4-{4-[1-(2,6-dioxopiperidin-3-yl)-3-methyl-2-oxo-1,3-benzodiazol-5-yl]piperazin-1-yl}cyclohexyl)-N-methylcarbamate (45 mg, 0.081 mmol) in DCM (1 mL) was added TFA (0.2 mL) dropwise at rt. The resulting mixture was stirred for 1 h at rt under nitrogen atmosphere. On completion, the reaction mixture was concentrated under reduced pressure. The residue was purified by trituration with Et₂O to give the title compound (54 mg) as a white solid. LC/MS (ESI, m/z): [(M+1)]⁺=455.2.

### Example 1.200. Preparation of 3-[5-(9-aminononyl)-3-methyl-2-oxo-1,3-benzodiazol-1-yl]piperidine-2,6-dione (Intermediate GU)

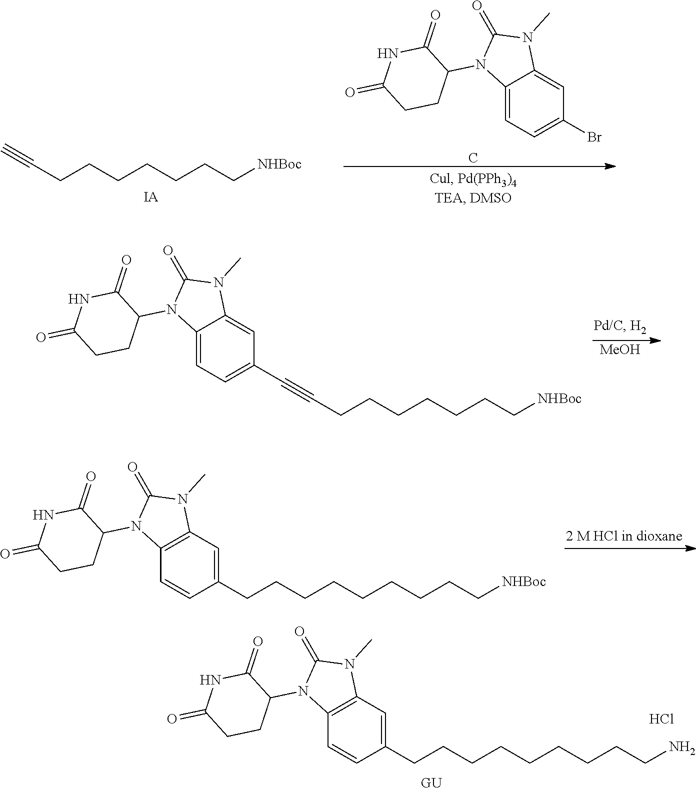

### Step 1—Tert-butyl N-[6-[1-(2,6-dioxopiperidin-3-yl)-3-methyl-2-oxo-1,3-benzodiazol-5-yl]hex-5-yn-1-yl]carbamate

To a stirred solution of 3-(5-bromo-3-methyl-2-oxo-1,3-benzodiazol-1-yl)piperidine-2,6-dione (8.00 g, 23.7 mmol, Intermediate C) and tert-butyl N-(hex-5-yn-1-yl)carbamate (9.33 g, 47.3 mmol, Intermediate IA) in DMSO (40.00 mL) and TEA (20.00 mL) were added Pd(PPh₃)₄ (2.73 g, 2.37 mmol) and CuI (450.55 mg, 2.366 mmol) in portions rt under nitrogen atmosphere. The resulting mixture was stirred for 3 h at 80° C. under nitrogen atmosphere. On completion, the reaction mixture was diluted with EtOAc (800 mL). The resulting mixture was washed with water (4×400 mL). The resulting mixture was concentrated under reduced pressure. The residue was purified by silica gel column chromatography, eluted with PE/EtOAc (1:2), to afford the title compound (7.2 g, 67% yield) as a yellow solid. ¹H NMR (400 MHz, DMSO-d₆) δ 11.09 (s, 1H), 7.23-7.21 (m, 1H), 7.10-7.06 (m, 2H), 6.75-6.72 (m, 1H), 5.36 (dd, J=12.8, 5.3 Hz, 1H), 3.30 (s, 3H), 2.92-2.83 (m, 3H), 2.75-2.55 (m, 2H), 2.40 (t, J=7.0 Hz, 2H), 2.06-1.98 (m, 1H), 1.57-1.50 (m, 2H), 1.43-1.34 (m, 13H), 1.31-1.23 (m, 4H). LC/MS (ESI, m/z): [(M+1-56)]⁺=497.3.

### Step 2—Tert-butyl N-[9-[1-(2,6-dioxopiperidin-3-yl)-3-methyl-2-oxo-1,3-benzodiazol-5-yl]nonyl]carbamate

To a stirred solution of tert-butyl N-[9-[1-(2,6-dioxopiperidin-3-yl)-3-methyl-2-oxo-1,3-benzodiazol-5-yl]non-8-yn-1-yl]carbamate (7.00 g, 14.1 mmol) in MeOH (500.00 mL) was added Pd/C (2.00 g, 1.88 mmol, 10 wt %) in portions at rt under nitrogen atmosphere. The resulting mixture was stirred for 24 h at rt under hydrogen atmosphere. On completion, the resulting mixture was filtered and the filter cake was washed with MeOH (3×100 mL). The filtrate was concentrated under reduced pressure. The residue was purified by reverse flash chromatography (column, C18 silica gel; mobile phase, ACN in water, 60% to 80% gradient in 25 min; detector, UV 254 nm) to afford the title compound (7 g) as a white solid. ¹H NMR (400 MHz, DMSO-d₆) δ 11.07 (s, 1H), 7.05-6.96 (m, 2H), 6.87-6.84 (m, 1H), 6.78-6.69 (m, 1H), 5.34 (dd, J=12.7, 5.4 Hz, 1H), 3.32 (s, 3H), 2.92-2.87 (m, 3H), 2.77-2.63 (m, 2H), 2.62-2.58 (m, 2H), 2.05-1.95 (m, 1H), 1.61-1.55 (m, 2H), 1.39-1.34 (s, 11H), 1.31-1.20 (m, 10H). LC/MS (ESI, m/z): [(M+1-56)]⁺=501.3.

### Step 3—3-[5-(9-aminononyl)-3-methyl-2-oxo-1,3-benzodiazol-1-yl]piperidine-2,6-dione Hydrochloride

To a stirred solution of tert-butyl N-[9-[1-(2,6-dioxopiperidin-3-yl)-3-methyl-2-oxo-1,3-benzodiazol-5-yl]nonyl]carbamate (6.00 g, 12.0 mmol) in DCM (15.00 mL) was added 4 M HCl (gas) in 1,4-dioxane (15.00 mL) dropwise at rt under nitrogen atmosphere. The resulting mixture was stirred for 4 h at rt under nitrogen atmosphere. On completion, the resulting mixture was concentrated under reduced pressure. The residue was triturated with Et₂O to afford the title compound (5 g, 96% yield) as an off-white solid. ¹H NMR (400 MHz, Methanol-d₄) δ 7.06-7.00 (m, 2H), 6.97-6.94 (m, 1H), 5.33 (dd, J=12.3, 5.4 Hz, 1H), 3.43 (s, 3H), 2.98-2.87 (m, 3H), 2.86-2.75 (m, 2H), 2.70 (t, J=7.6 Hz, 2H), 2.21-2.15 (m, 1H), 1.75-1.59 (m, 4H), 1.45-1.30 (m, 10H). LC/MS (ESI, m/z): [(M+1-56)]⁺=401.3.

### Example 1.201. Preparation of 3-{4-[Azetidin-3-yl(methyl)amino]-3-methyl-2-oxo-1,3-benzodiazol-1-yl}piperidine-2,6-dione (Intermediate GV)

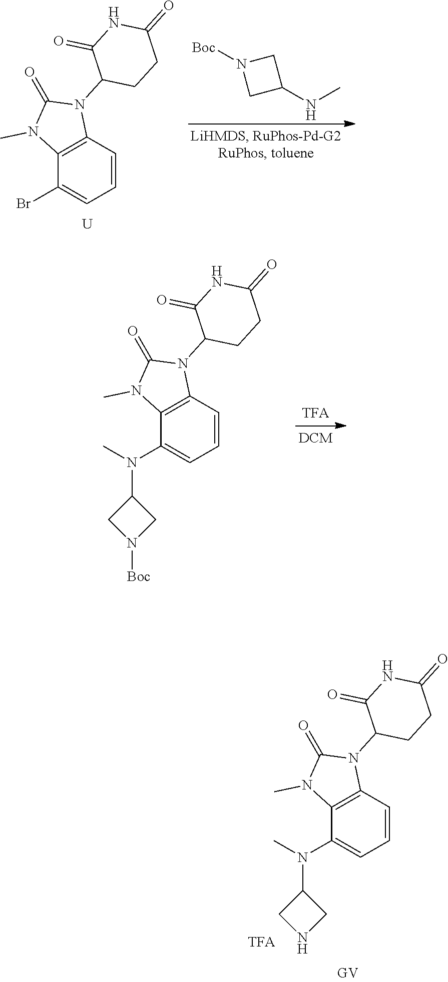

### Step 1—Tert-butyl 3-{[1-(2,6-dioxopiperidin-3-yl)-3-methyl-2-oxo-1,3-benzodiazol-4-yl](methyl)amino}azetidine-1-carboxylate

To a stirred solution of 3-(4-bromo-3-methyl-2-oxo-1,3-benzodiazol-1-yl)piperidine-2,6-dione (600 mg, 2 mmol, Intermediate U) and tert-butyl 3-(methylamino)azetidine-1-carboxylate (495.710 mg, 2.661 mmol, CAS #454703-20-9) in toluene (10 mL) were added RuPhos-PdCl-2nd G (275.990 mg, 0.355 mmol), RuPhos (165.590 mg, 0.355 mmol) followed by addition LiHMDS (10.644 mL, 10.644 mmol) at nitrogen atmosphere. The resulting mixture was stirred under nitrogen atmosphere at 80° C. for 2 h. On completion, the reaction mixture was cooled to rt and was acidified to pH 3 with FA. The resulting mixture was concentrated under reduced pressure. The residue was dissolved in DMSO (20 mL) and was purified by reverse phase flash chromatography (Column: WelFlash™ C18-I, 20-40 um, 330 g; Eluent A: Water (plus 10 mmol/L FA); Eluent B: ACN; Gradient: 20%-50% B in 25 min; Flow rate: 80 mL/min; Detector: 220/254 nm; desired fractions were collected at 36% B) and concentrated under reduced pressure to afford the title compound (430 mg, 55%) as a brown solid. LC/MS (ESI, m/z): [(M+H)]⁺=444.2.

### Step 2—3-{4-[Azetidin-3-yl(methyl)amino]-3-methyl-2-oxo-1,3-benzodiazol-1-yl}piperidine-2,6-dione Trifluoroacetate

To a stirred solution of tert-butyl 3-{[1-(2,6-dioxopiperidin-3-yl)-3-methyl-2-oxo-1,3-benzodiazol-4-yl](methyl)amino}azetidine-1-carboxylate (430 mg, 0.97 mmol) in DCM (9 mL) was added TFA (3 mL) dropwise at rt. The resulting mixture was stirred for 1 h at rt. On completion, the reaction mixture was concentrated under reduced pressure. The residue was triturated with Et₂O (15 mL) to afford the title compound (412 mg) as a brown solid. LC/MS (ESI, m/z): [(M+H)]⁺=344.1.

### Example 1.202. Preparation of 3-{3-methyl-5-[4-(methylamino)piperidin-1-yl]-2-oxo-1,3-benzodiazol-1-yl}piperidine-2,6-dione Trifluoroacetate (Intermediate GW)

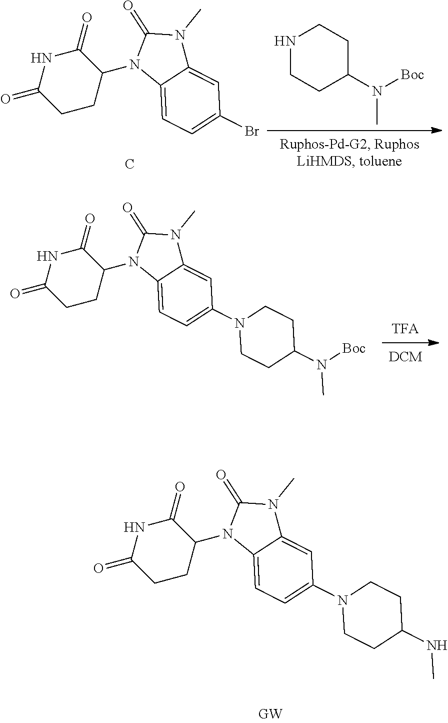

### Step 1—Tert-butyl (1-(1-(2,6-dioxopiperidin-3-yl)-3-methyl-2-oxo-2,3-dihydro-1H-benzo[d]imidazol-5-yl)piperidin-4-yl)(methyl)carbamate

To a solution of 3-(5-bromo-3-methyl-2-oxo-benzimidazol-1-yl)piperidine-2,6-dione (6, 17.74 mmol, Intermediate C), tert-butyl N-methyl-N-(4-piperidyl)carbamate (4.56 g, 21.2 mmol, CAS #108612-54-0), [2-(2-aminophenyl)phenyl]-chloro-palladium; dicyclohexyl-[2-(2,6-diisopropoxyphenyl)phenyl]phosphane (2.76 g, 3.55 mmol), dicyclohexyl-[2-(2,6-diisopropoxyphenyl)phenyl]phosphane (1.66 g, 3.55 mmol) and 4 Å molecular sieves (1.2 g) in toluene (120 mL) was added LiHMDS (1 M, 88.72 mL). The mixture was stirred at 80° C. under N₂ for 2 hr. On completion, the reaction mixture was adjusted to pH=5 with FA at 0° C., then filtered, and the filtrate was concentrated under reduced pressure to give a residue. The residue was purified by column chromatography (SiO₂, PE:EA=2:1 to 0:1, P: Rf=0.4 (PE:EA=0:1)) to give the crude product (6 g, 85% purity). The crude product (6 g, 85% purity) was triturated with MTBE (20 mL) at 25° C. for 10 min and filtered, then washed ACN (10 mL×3), then filtered and dried in vacuo to give the title compound (4.66 g, 52% yield) as off-white solid. ¹H NMR (400 MHz, DMSO-d6) δ=11.06 (s, 1H), 6.94-6.92 (d, J=8.7 Hz, 1H), 6.84 (s, 1H), 6.66-6.63 (d, J=2.1 Hz, 1H), 5.31-5.26 (dd, J=5.3, 12.9 Hz, 1H), 4.02-3.91 (m, 1H), 3.68-1.65 (br d, J=11.9 Hz, 2H), 3.30 (s, 3H), 2.92-2.89 (m, 1H), 2.74-2.60 (m, 7H), 1.99-1.98 (m, 1H), 1.82-1.78 (m, 2H), 1.63-1.61 (m, 2H), 1.41 (s, 9H).

### Step 2—3-{3-methyl-5-[4-(methylamino)piperidin-1-yl]-2-oxo-1,3-benzodiazol-1-yl}piperidine-2,6-dione Trifluoroacetate

To a stirred solution of tert-butyl N-{1-[1-(2,6-dioxopiperidin-3-yl)-3-methyl-2-oxo-1,3-benzodiazol-5-yl]piperidin-4-yl}-N-methylcarbamate (900 mg, 1.91 mmol) in DCM was added TFA (6 mL) dropwise in portions at rt. The resulting mixture was stirred for 1 h rt. On completion, the resulting mixture was concentrated under reduced pressure. The residue was purified by trituration with diethyl ether (50 mL) to afford the title compound (600 mg) as a yellow solid. LC/MS (ESI, m/z): [(M+H)]⁺=372.2.

### Example 1.203. Preparation of tert-butyl 7-(5-bromo-2-pyridyl)-2,7-diazaspiro[3.5]nonane-2-carboxylate (Intermediate GX)

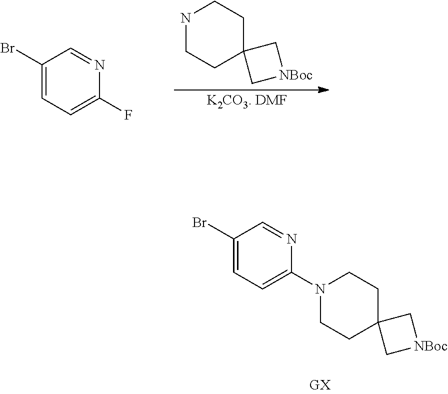

To a solution of 5-bromo-2-fluoro-pyridine (3.00 g, 17.0 mmol, 1.75 mL) in dimethylformamide (50 mL) was added potassium carbonate (4.71 g, 34.1 mmol) and tert-butyl 2,7-diazaspiro[3.5]nonane-2-carboxylate (3.86 g, 17.0 mmol, CAS #236406-55-6). The mixture was stirred at 80° C. for 12 hours. On completion, the reaction mixture was quenched by addition water (50 mL) at 25° C., and then extracted with ethyl acetate (40 mL×3). The combined organic layers were washed with brine (50 mL), dried over anhydrous sodium sulfate, filtered and concentrated under reduced pressure to give a residue. The residue was purified by flash silica gel chromatography (PE:EA=1:0 to 3:1) to give the title compound (6 g, 92% yield) as a white solid. ¹H NMR (400 MHz, DMSO-d₆) δ=8.13 (d, J=2.4 Hz, 1H), 7.63 (dd, J=2.8, 9.2 Hz, 1H), 6.84 (d, J=8.8 Hz, 1H), 3.58 (s, 4H), 3.46 (s, 4H), 1.67 (t, J=5.6 Hz, 4H), 1.38 (s, 9H).

### Example 1.204. Preparation of 3-(5-(6-(2,7-diazaspiro[3.5]nonan-7-yl)pyridin-3-yl)-3-methyl-2-oxo-2,3-dihydro-1H-benzo[d]imidazol-1-yl)piperidine-2,6-dione (Intermediate GY)

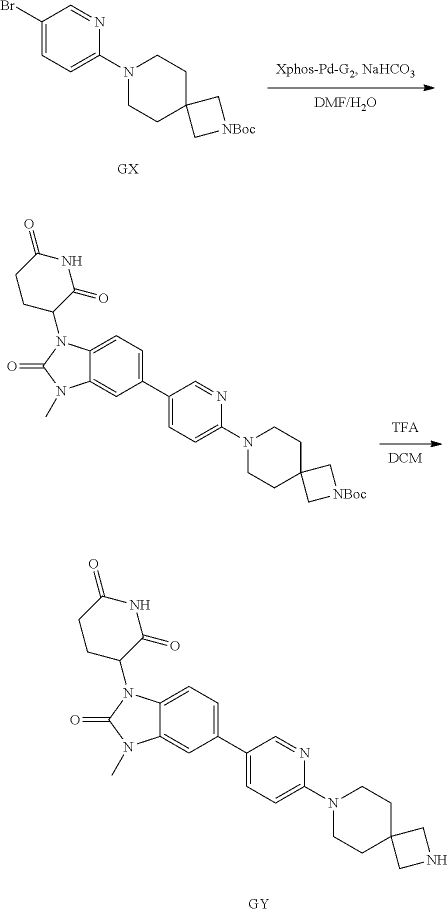 [SMILES: CC(C)(C)OC(=O)N1CC2(CCN(c3ccc(Br)cn3)CC2)C1 ; Cn1c(=O)n(C2CCC(=O)NC2=O)c2ccc(-c3ccc(N4CCC5(CC4)CN(C(=O)OC(C)(C)C)C5)nc3)cc21 ; Cn1c(=O)n(C2CCC(=O)NC2=O)c2ccc(-c3ccc(N4CCC5(CC4)CNC5)nc3)cc21]

### Step 1—Tert-butyl 7-[5-[1-(2,6-dioxo-3-piperidyl)-3-methyl-2-oxo-benzimidazol-5-yl]-2-pyridyl]-2,7-diazaspiro[3.5]nonane-2-carboxylate

A mixture of tert-butyl 7-(5-bromo-2-pyridyl)-2,7-diazaspiro[3.5]nonane-2-carboxylate (3.87 g, 10.1 mmol, Intermediate GX) 3-[3-methyl-2-oxo-5-(4,4,5,5-tetramethyl-1,3,2-dioxaborolan-2-yl)benzimidazol-1-yl]piperidine-2,6-dione (3.00 g, 7.79 mmol, Intermediate Y), chloro(2-dicyclohexylphosphino-2′,4′,6′-triisopropyl-1,1′-biphenyl)[2-(2′-amino-1,1′-biphenyl)]palladium(II) (612 mg, 778 umol) and sodium bicarbonate (1.31 g, 15.5 mmol) in dimethylformamide (50 mL) and water (5 mL) was degassed and purged with nitrogen three times. Then the mixture was stirred at 80° C. for 4 hours under nitrogen atmosphere. On completion, the reaction mixture was quenched by addition water (50 mL) at 25° C., and extracted with EA (100 mL*3). The combined organic layers were washed with brine (100 mL), dried over anhydrous sodium sulfate, filtered and concentrated under reduced pressure to give a residue. The crude product was purified by reversed-phase HPLC (0.1% FA condition) to give the title compound (1.98 g, 42% yield) as a white solid. ¹H NMR (400 MHz, DMSO-d₆) δ=11.12 (s, 1H), 8.46 (d, J=2.4 Hz, 1H), 7.85 (dd, J=2.4, 9.2 Hz, 1H), 7.45 (d, J=1.2 Hz, 1H), 7.28 (dd, J=1.6, 8.0 Hz, 1H), 7.16 (d, J=8.0 Hz, 1H), 6.94 (d, J=9.2 Hz, 1H), 5.39 (dd, J=5.6, 12.8 Hz, 1H), 3.61 (s, 4H), 3.54 (s, 4H), 3.40 (s, 3H), 2.98-2.85 (m, 1H), 2.80-2.73 (m, 1H), 2.69-2.60 (m, 2H), 2.07-2.00 (m, 1H), 1.72 (t, J=5.2 Hz, 4H), 1.39 (s, 9H); LC-MS (ESI+) m/z 561.1 (M+H)⁺.

### Step 2—3-(5-(6-(2,7-diazaspiro[3.5]nonan-7-yl)pyridin-3-yl)-3-methyl-2-oxo-2,3-dihydro-1H-benzo[d]imidazol-1-yl)piperidine-2,6-dione Trifluoroacetate

To a mixture of tert-butyl 7-(5-(1-(2,6-dioxopiperidin-3-yl)-3-methyl-2-oxo-2,3-dihydro-1H-benzo[d]imidazol-5-yl)pyridin-2-yl)-2,7-diazaspiro[3.5]nonane-2-carboxylate (50 mg, 0.089 mmol) in DCM (2 mL) was added TFA (1 mL) dropwise. The resulting mixture was stirred for 1 h at rt under nitrogen atmosphere. On completion, the reaction mixture was concentrated under reduced pressure. The residue was triturated with Et₂O to afford the title compound (40 mg, 97% yield) as a brown solid. LC/MS (ESI, m/z): [(M+H)]⁺=461.2.

### Example 1.205. Preparation of tert-butyl 4-((6-(5-iodopyrimidin-2-yl)-1,6-diazaspiro(3.3)heptan-1-yl)methyl)piperidine-1-carboxylate (Intermediate GZ)

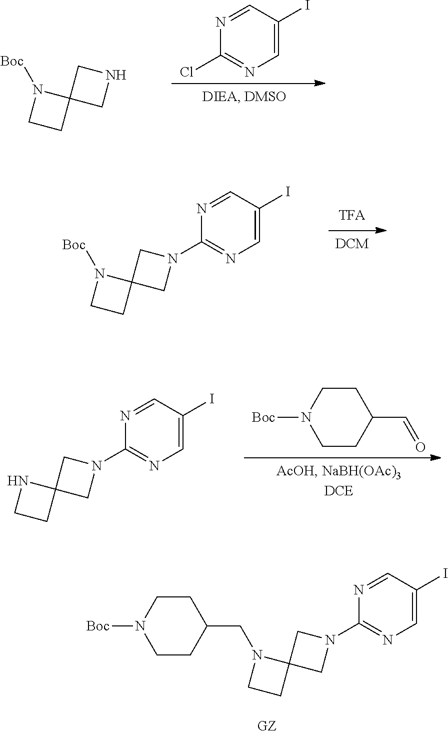 [SMILES: CC(C)(C)OC(=O)N1CCC12CNC2 ; Clc1ncc(I)cn1 ; CC(C)(C)OC(=O)N1CCC12CN(c1ncc(I)cn1)C2 ; Ic1cnc(N2CC3(CCN3)C2)nc1 ; CC(C)(C)OC(=O)N1CCC(C=O)CC1 ; CC(C)(C)OC(=O)N1CCC(CN2CCC23CN(c2ncc(I)cn2)C3)CC1]

### Step 1—Tert-butyl 6-(5-iodopyrimidin-2-yl)-1,6-diazaspiro(3.3)heptane-1-carboxylate

To a solution of tert-butyl 1,6-diazaspiro[3.3]heptane-1-carboxylate (5 g, 30 mmol, CAS #1330763-95-5) and 2-chloro-5-iodo-pyrimidine (7.28 g, 30.3 mmol) in DMSO (50 mL) was added DIEA (16.3 g, 126 mmol, 22 mL). The mixture was stirred at 100° C. for 10 h. On completion, the reaction mixture was poured into ice water (50 mL) and extracted with EtOAc (100 mL×3). The combined organic layers were washed with brine (100 mL×3), dried over sodium sulfate, filtered and concentrated under reduced pressure to give a residue. The residue was purified by flash silica gel chromatography (ISCO®; 40 g SepaFlash® Silica Flash Column, Eluent of 0˜20% Ethyl acetate/Petroleum ethergradient @ 80 mL/min) to give the title compound (6.5 g, 64% yield) as a yellow solid. LC-MS (ESI+) m/z 403.1 (M+H)⁺.

### Step 2—6-(5-Iodopyrimidin-2-yl)-1,6-diazaspiro(3.3)heptane

To a solution of tert-butyl 6-(5-iodopyrimidin-2-yl)-1,6-diazaspiro[3.3]heptane-1-carboxylate (3 g, 8 mmol) in DCM (30 mL) was added TFA (6 mL) and the mixture was stirred at 25° C. for 4 h. On completion, The reaction was poured into NaHCO₃ (sat. aq. 30 mL) and extracted with DCM (60 mL×3). The combined organic phase is washed with brine (60 mL×3), then dried over sodium sulfate. The mixture was filtered, then the filtrate was concentrated to give the title compound (3.6 g) as a yellow solid. ¹H NMR (400 MHz, DMSO-d₆) δ=8.54 (s, 2H), 4.39 (d, J=10.4 Hz, 2H), 4.24 (d, J=10.4 Hz, 2H), 3.71 (t, J=8.2 Hz, 2H), 2.66 (t, J=8.2 Hz, 2H).

### Step 3—Tert-butyl 4-((6-(5-iodopyrimidin-2-yl)-1,6-diazaspiro(3.3)heptan-1-yl)methyl)piperidine-1-carboxylate

To a solution of 6-(5-iodopyrimidin-2-yl)-1,6-diazaspiro[3.3]heptane (3.6 g, 11.9 mmol) and tert-butyl 4-formylpiperidine-1-carboxylate (2.54 g, 11.9 mmol) in DCE (40 mL) was added AcOH (2.15 g, 35.8 mmol, 2.04 mL), then NaBH(OAc)₃ (7.58 g, 35.8 mmol) was added at 0° C. The mixture was stirred at 25° C. for 2 h. On completion. The reaction mixture was poured into ice water (40 mL) and extracted with DCM (80 mL×3), the combined organic layers were washed with brine (80 mL×3), dried over sodium sulfate, filtered and concentrated under reduced pressure to give a residue. The crude product was purified by re-crystallization from EtOAc (15 mL) at 25° C. to give the title compound (3.3 g, 56% yield) as a yellow solid. ¹H NMR (400 MHz, DMSO-d₆) δ 8.51 (br s, 2H), 4.73-4.40 (m, 1H), 4.25-3.94 (m, 3H), 3.88 (br d, J=13.8 Hz, 2H), 3.17-2.98 (m, 1H), 2.70-2.58 (m, 2H), 2.41-2.25 (m, 2H), 1.76-1.60 (m, 2H), 1.37 (s, 10H), 0.98 (br s, 2H).

### Example 1.206. Preparation of 3-(3-methyl-2-oxo-5-((2-(1-(piperidin-4-ylmethyl)-1,6-diazaspiro[3.3]heptan-6-yl)pyrimidin-5-yl)ethynyl)-2,3-dihydro-1H-benzo[d]imidazol-1-yl)piperidine-2,6-dione (Intermediate HA)

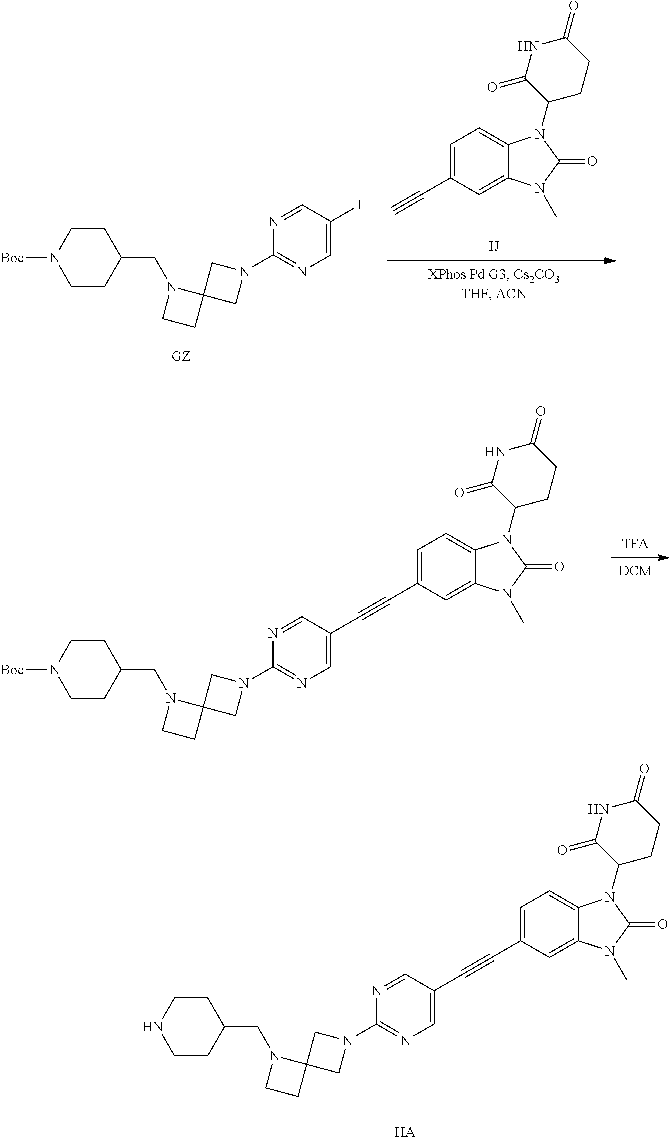

### Step 1—Tert-butyl 4-((6-(5-((1-(2,6-dioxopiperidin-3-yl)-3-methyl-2-oxo-2,3-dihydro-1H-benzo(d)imidazol-5-yl)ethynyl)pyrimidin-2-yl)-1,6-diazaspiro(3.3)heptan-1-yl)methyl)piperidine-1-carboxylate

To a solution of tert-butyl 4-[[6-(5-iodopyrimidin-2-yl)-1,6-diazaspiro[3.3]heptan-1-yl]methyl]piperidine-1-carboxylate (2.8 g, 5.61 mmol, Intermediate GZ) and 3-(5-ethynyl-3-methyl-2-oxo-benzimidazol-1-yl)piperidine-2,6-dione (3.18 g, 11.2 mmol, Intermediate IJ) in THF (20 mL) and ACN (20 mL) was added XPhos Pd G3 (474 mg, 561 umol) and Cs₂CO₃ (5.48 g, 16.8 mmol). The mixture was stirred at 60° C. for 10 h under N₂ atmosphere. On completion, the reaction mixture was poured into NH₄Cl (sat,aq, 40 mL) and extracted with EtOAc (80 mL×3). The combined organic layers were washed with brine (80 mL×3), dried over sodium sulfate, filtered and concentrated under reduced pressure to give a residue. The residue was purified by flash silica gel chromatography (0˜100% Ethyl acetate/Petroleum ether gradient @80 mL/min) to give the title compound (1.27 g, 32% yield) as a yellow solid. ¹H NMR (400 MHz, DMSO-d₆) δ 11.12 (s, 1H), 8.51 (s, 2H), 7.38 (d, J=1.4 Hz, 1H), 7.27-7.20 (m, 1H), 7.19-7.13 (m, 1H), 5.40 (dd, J=5.4, 12.8 Hz, 1H), 4.24 (br d, J=10.4 Hz, 2H), 4.07 (d, J=9.6 Hz, 2H), 3.89 (br d, J=12.4 Hz, 2H), 3.36 (s, 3H), 3.07 (br t, J=6.4 Hz, 2H), 2.96-2.83 (m, 1H), 2.71-2.60 (m, 3H), 2.42 (br d, J=6.6 Hz, 2H), 2.29 (br t, J=6.4 Hz, 2H), 2.10-2.00 (m, 1H), 1.67 (br d, J=11.2 Hz, 2H), 1.37 (s, 11H), 1.03-0.91 (m, 2H). LC-MS (ESI+) m/z 655.1 (M+H)⁺.

### Step 2—3-(3-Methyl-2-oxo-5-((2-(1-(piperidin-4-ylmethyl)-1,6-diazaspiro[3.3]heptan-6-yl)pyrimidin-5-yl)ethynyl)-2,3-dihydro-1H-benzo[d]imidazol-1-yl)piperidine-2,6-dione

To a solution of tert-butyl 4-[[6-[5-[2-[1-(2,6-dioxo-3-piperidyl)-3-methyl-2-oxo-benzimidazol-5-yl]ethynyl]pyrimidin-2-yl]-1,6-diazaspiro[3.3]heptan-1-yl]methyl]piperidine-1-carboxylate (100 mg, 153 umol) in DCM (1 mL) and TFA (0.2 mL). The mixture was stirred at 25° C. for 4 h. On completion, the reaction mixture was concentrated under reduced pressure to give a residue. The residue was purified by prep-HPLC (column: Phenomenex luna C18 150*25 mm*10 um; mobile phase: [water (FA)-ACN]; B %: 1%-28%, 10.5 min) to give the title compound (15 mg, 17% yield) as a yellow solid. LC-MS (ESI⁺) m/z 555.6 (M+H)⁺; ¹H NMR (400 MHz, DMSO-d₆) δ 11.16-11.02 (m, 1H), 8.52 (s, 2H), 8.32 (s, 1H), 7.38 (s, 1H), 7.25-7.21 (m, 1H), 7.19-7.16 (m, 1H), 5.40 (dd, J=5.6, 12.8 Hz, 1H), 4.25 (d, J=9.6 Hz, 2H), 4.07 (d, J=10.4 Hz, 2H), 3.16-3.07 (m, 8H), 2.35-2.27 (m, 9H), 2.09-1.97 (m, 2H), 1.81-1.74 (m, 2H), 1.53-1.44 (m, 1H), 1.20-1.11 (m, 2H).

### Example 1.207. Preparation of tert-butyl N-[(5-bromo-2-pyridyl)methyl]-N-methyl-carbamate (Intermediate HB)

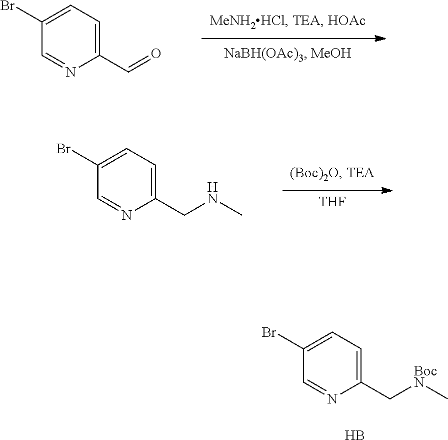

### Step 1—1-(5-Bromo-2-pyridyl)-N-methyl-methanamine

To a solution of methanamine; hydrochloride (14.5 g, 215 mmol) in methanol (70 mL) was added triethylamine (4.35 g, 43.0 mmol) and the reaction mixture was stirred at 25° C. for 1 hour. To the resulting reaction mixture was added 5-bromopyridine-2-carbaldehyde (8 g, 43.01 mmol), then the reaction was stirred at 25° C. for 0.5 hour. To the resulting reaction mixture was added sodium triacethoxy borohydride (18.2 g, 86.0 mmol) and acetic acid (5.17 g, 86.0 mmol) and stirred at 25° C. for 11 hour. On completion, the reaction mixture was diluted with water (50 mL), and extracted with ethyl acetate (30 mL×3), dried over anhydrous sodium sulfate, concentrated under reduced pressure to give a residue. The crude product was purified by reversed-phase HPLC (neutral condition) to give the title compound (4 g, 34% yield) as a white solid. ¹H NMR (400 MHz, DMSO-d₆) δ=8.61 (s, 1H), 8.01-7.99 (m, 1H), 7.41 (d, J=8.4 Hz, 1H), 3.73 (s, 2H), 2.29 (s, 3H).

### Step 2—Tert-butyl N-[(5-bromo-2-pyridyl)methyl]-N-methyl-carbamate

A mixture of 1-(5-bromo-2-pyridyl)-N-methyl-methanamine (2.8 g, 13.9 mmol), di-tert-butyl dicarbonate (7.60 g, 34.8 mmol), triethylamine (7.05 g, 69.6 mmol) in tetrahydrofuran (150 mL) was degassed and purged with nitrogen three times. Then the mixture was stirred at 25° C. for 12 h under nitrogen atmosphere. The reaction mixture was diluted with water (50 mL), and extracted with EA (100 mL×3), dried over anhydrous sodium sulfate, concentrated under reduced pressure to give a residue. The crude was purified by silica gel column chromatography (Petroleum ether/Ethyl acetate=2:1) to give the title compound (2.6 g, 61% yield) as a white solid. LCMS=323.0[M+Na]⁺.

### Example 1.208. Preparation of 3-(3-methyl-5-{6-[(methylamino)methyl]pyridin-3-yl}-2-oxo-1,3-benzodiazol-1-yl)piperidine-2,6-dione (Intermediate HC)

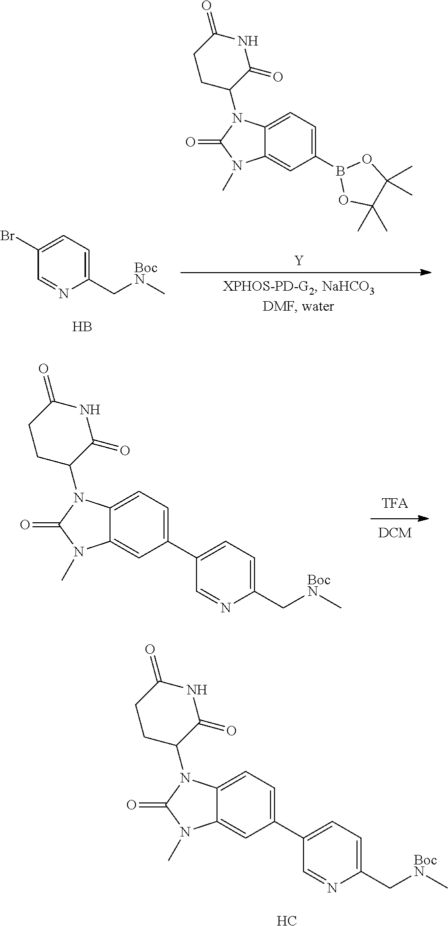

### Step 1—Tert-butyl N-[[5-[1-(2,6-dioxo-3-piperidyl)-3-methyl-2-oxo-benzimidazol-5-yl]-2-pyridyl]methyl]-N-methyl-carbamate

A mixture of tert-butyl N-[(5-bromo-2-pyridyl)methyl]-N-methyl-carbamate (2.6 g, 8.63 mmol, Intermediate HB), 3-[3-methyl-2-oxo-5-(4,4,5,5-tetramethyl-1,3,2-dioxaborolan-2-yl)benzimidazol-1-yl]piperidine-2,6-dione (3.33 g, 8.63 mmol, Intermediate Y), chloro(2-dicyclohexylphosphino-2′,4′,6′-triisopropyl-1,1′-biphenyl)[2-(2′-amino-1,1′-biphenyl)]palladium(II) (679 mg, 0.86 mmol), sodium bicarbonate (1.45 g, 17.2 mmol) in dimethylformamide (40 mL) and water (4 mL) was degassed and purged with nitrogen three times. Then the mixture was stirred at 80° C. for 2 hours under nitrogen atmosphere. On completion, the reaction mixture was diluted with water (40 mL), and extracted with ethyl acetate (100 mL×3), dried over anhydrous sodium sulfate, concentrated under reduced pressure to give a residue. The crude product was purified by reversed-phase HPLC (0.1% FA condition) to give the title compound (1.1 g, 22% yield) as a white solid. ¹H NMR (400 MHz, DMSO-d₆) δ=10.96-10.74 (m, 1H), 8.85 (d, J=2.0 Hz, 1H), 8.07 (dd, J=2.4, 8.0 Hz, 1H), 7.53 (d, J=1.6 Hz, 1H), 7.39 (dd, J=1.6, 8.4 Hz, 1H), 7.28 (d, J=8.0 Hz, 1H), 7.21 (d, J=8.4 Hz, 1H), 5.39-5.32 (m, 1H), 4.51 (s, 2H), 3.42 (s, 3H), 2.90 (s, 3H), 2.82-2.62 (m, 3H), 2.14-2.06 (m, 1H), 1.42 (s, 9H); LCMS=480.3[M+H]⁺.

### Step 2—3-(3-Methyl-5-{6-[(methylamino)methyl]pyridin-3-yl}-2-oxo-1,3-benzodiazol-1-yl)piperidine-2,6-dione Trifluoroacetate

To a stirred mixture of tert-butyl N-({5-[1-(2,6-dioxopiperidin-3-yl)-3-methyl-2-oxo-1,3-benzodiazol-5-yl]pyridin-2-yl}methyl)-N-methylcarbamate (50.00 mg, 0.104 mmol) in DCM (3 mL) was added TFA (0.6 mL) at rt. The resulting mixture was stirred for 1 h at rt. On completion, the reaction mixture was concentrated under reduced pressure. The residue was triturated with Et₂O to afford the title compound (56.0 mg) as a white solid. LC/MS (ESI, m/z): [(M+H)]⁺=380.1.

### Example 1.209. Preparation of tert-butyl 4-[(4-{5-[1-(2,6-dioxopiperidin-3-yl)-3-methyl-2-oxo-1,3-benzodiazol-5-yl]pyridin-2-yl}-1,4-diazepan-1-yl)methyl]piperidine-1-carboxylate (Intermediate HD)

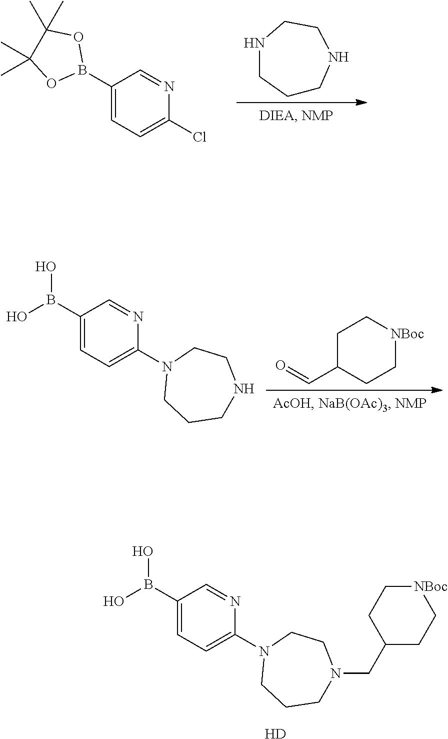

### Step-1—(6-(1,4-Diazepan-1-yl)pyridin-3-yl)boronic Acid

To a solution of 2-chloro-5-(4,4,5,5-tetramethyl-1,3,2-dioxaborolan-2-yl) pyridine (10 g, 41.7 mmol, CAS #444120-94-9) and 1, 4-diazepane (20.9 g, 208 mmol, CAS #505-66-8) in 1-methylpyrrolidin-2-one (160 mL) was added N-ethyl-N-propan-2-ylpropan-2-amine (26.9 g, 208 mmol). The mixture was stirred at 120° C. for 12 hours. On completion, the reaction mixture was partitioned between water (200 mL) and ethyl acetate (600 mL). The organic phase was separated, dried over with anhydrous sodium sulfate, filtered and concentrated under reduced pressure to give the title compound (3.2) as a yellow oil. LC-MS (ESI+) m/z 222.0 (M+H)⁺.

### Step 2—(6-(4-((1-(Tert-butoxycarbonyl)piperidin-4-yl)methyl)-1,4-diazepan-1-yl)pyridin-3-yl)boronic Acid

To a solution of [6-(1,4-diazepan-1-yl)-3-pyridyl]boronic acid (3.11 g, 14.0 mmol) and tert-butyl 4-formylpiperidine-1-carboxylate (3 g, 14.0 mmol, CAS #137076-22-3) in 1-methylpyrrolidin-2-one (100 mL) was added acetic acid (844 mg, 14.0 mmol) and sodium triacethoxy borohydride (5.96 g, 28.1 mmol). The mixture was stirred at 25° C. for 12 hours. On completion, the reaction mixture was filtered and concentrated under reduced pressure to give a residue. The crude product was purified by reversed-phase HPLC (0.1% NH₃·H₂O) to give the title compound (2 g, 21% yield) as a yellow gum. LC-MS (ESI+) m/z 419.2 (M+H)⁺.

### Example 1.210. Preparation of 3-(3-Methyl-2-oxo-5-{6-[4-(piperidin-4-ylmethyl)-1,4-diazepan-1-yl]pyridin-3-yl}-1,3-benzodiazol-1-yl)piperidine-2,6-dione (Intermediate HE)

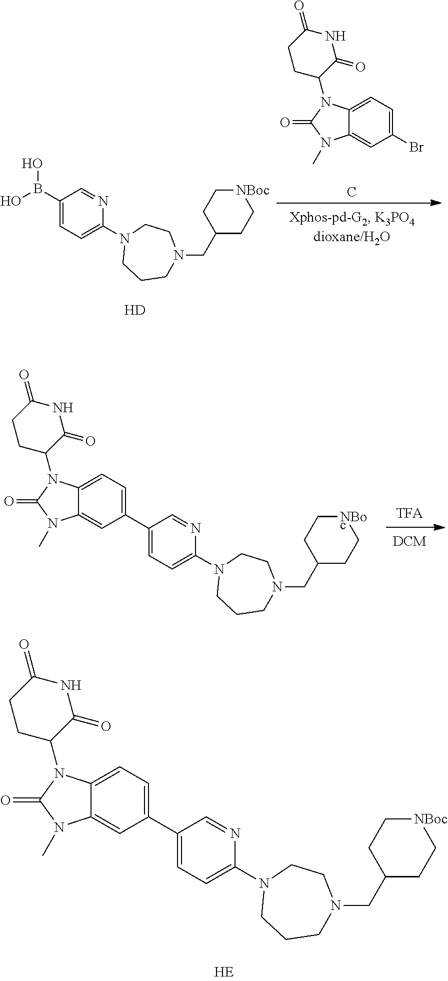

### Step 1—Tert-butyl 4-((4-(5-(1-(2,6-dioxopiperidin-3-yl)-3-methyl-2-oxo-2,3-dihydro-1H-benzo[d]imidazol-5-yl)pyridin-2-yl)-1,4-diazepan-1-yl)methyl)piperidine-1-carboxylate

To a solution of [6-[4-[(1-tert-butoxycarbonyl-4-piperidyl)methyl]-1, 4-diazepan-1-yl]-3-pyridyl]boronic acid (1.9 g, 4.5 mmol, Intermediate HD) and 3-(5-bromo-3-methyl-2-oxo-benzimidazol-1-yl)piperidine-2,6-dione (1.54 g, 4.54 mmol, Intermediate C) in dioxane (40 mL) and water (4 mL) was added chloro (2-dicyclohexylphosphino-2′,4′,6′-triisopropyl-1,1′-biphenyl)[2-(2′-amino-1,1′-biphenyl)]palladium (II) (357 mg, 454 μmol) and potassium phosphate (1.93 g, 9.08 mmol). The mixture was stirred at 60° C. for 12 hours. On completion, the reaction mixture was partitioned between water (50 mL) and ethyl acetate (200 mL). The organic phase was separated, dried over with anhydrous sodium sulfate, filtered and concentrated under reduced pressure to give a residue. The crude product was purified by reversed-phase HPLC (0.1% FA condition) to give the title compound (1.32 g, 42% yield) as a white solid. ¹H NMR (400 MHz, DMSO-d₆) δ=11.12 (s, 1H), 8.42 (d, J=2.4 Hz, 1H), 7.82 (dd, J=2.4, 8.8 Hz, 1H), 7.43 (d, J=1.6 Hz, 1H), 7.27 (dd, J=1.6, 8.4 Hz, 1H), 7.15 (d, J=8.4 Hz, 1H), 6.71 (d, J=8.8 Hz, 1H), 5.39 (dd, J=5.6, 12.8 Hz, 1H), 3.89 (d, J=11.6 Hz, 2H), 3.74 (d, J=4.8 Hz, 2H), 3.65 (t, J=6.0 Hz, 2H), 3.40 (s, 3H), 2.97-2.87 (m, 1H), 2.76-2.55 (m, 8H), 2.36-2.27 (m, 2H), 2.08-2.00 (m, 1H), 1.90-1.79 (m, 2H), 1.69-1.55 (m, 3H), 1.37 (s, 9H), 0.99-0.81 (m, 2H); LC-MS (ESI+) m/z 632.2 (M+H)⁺.

### Step 2—3-(3-Methyl-2-oxo-5-{6-[4-(piperidin-4-ylmethyl)-1,4-diazepan-1-yl]pyridin-3-yl}-1,3-benzodiazol-1-yl)piperidine-2,6-dione Trifluoroacetate

To a stirred solution of tert-butyl 4-[(4-{5-[1-(2,6-dioxopiperidin-3-yl)-3-methyl-2-oxo-1,3-benzodiazol-5-yl]pyridin-2-yl}-1,4-diazepan-1-yl)methyl]piperidine-1-carboxylate (45 mg, 0.071 mmol) in DCM (1 mL) was added TFA (0.2 mL, 2.693 mmol) dropwise at rt. The resulting mixture was stirred for 1 h at rt. On completion, the reaction mixture was concentrated under reduced pressure. The residue was purified by trituration with diethyl ether (5 mL). The resulting mixture was concentrated under reduced pressure to afford the title compound (44 mg, 96% yield) as a white solid. LC/MS (ESI, m/z): [(M+1)]⁺=532.3.

### Example 1.211. Preparation of tert-butyl 4-((1r,3r)-3-(4-(prop-2-yn-1-yloxy)piperidin-1-yl)cyclobutanecarbonyl)piperazine-1-carboxylate (Intermediate HF) & tert-butyl 4-((1s,3s)-3-(4-(prop-2-yn-1-yloxy)piperidin-1-yl)cyclobutanecarbonyl)piperazine-1-carboxylate (Intermediate IM′)

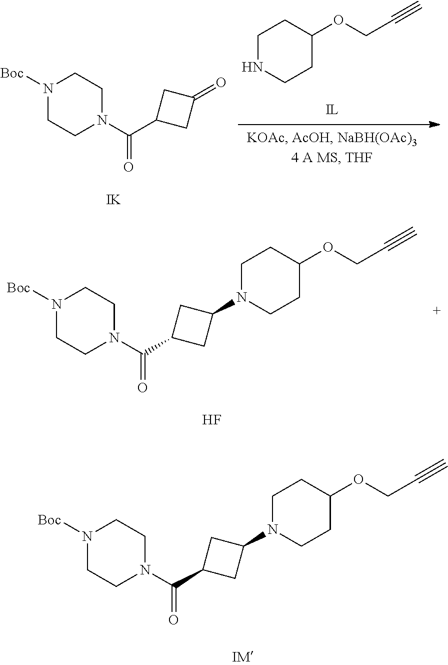

To a solution of tert-butyl 4-(3-oxocyclobutanecarbonyl)piperazine-1-carboxylate (5 g, 20 mmol, Intermediate IK) in THF (50 mL) was added 4-(prop-2-yn-1-yloxy)piperidine (2.47 g, 17.7 mmol, Intermediate IL), AcOH (3.19 g, 53.1 mmol, 3.04 mL), KOAc (5.21 g, 53.1 mmol) and 4 Å molecular sieves (4 g, 17.7 mmol), and it was stirred at 0° C. for 30 mim. Then, NaBH(OAc)₃ (11.3 g, 53.1 mmol) was added and the mixture was stirred at 20° C. for 12 h. On completion, the reaction mixture was quenched with NH₄Cl (50 mL) and extracted with EtOAc (60 mL×2). The combined organic layers were washed with brine (45 mL×3), dried over Na₂SO₄ and evaporated. The residue was purified by reversed-phase HPLC (water (FA)-ACN]; B %: 5%-45%, 30 min) to give tert-butyl 4-((1r,3r)-3-(4-(prop-2-yn-1-yloxy)piperidin-1-yl)cyclobutanecarbonyl)piperazine-1-carboxylate (1.83 g, 26% yield) as a white solid (¹H NMR (400 MHz, MeOD) δ 4.24 (d, J=2.4 Hz, 2H), 3.94-3.84 (m, 1H), 3.64-3.53 (m, 3H), 3.48-3.38 (m, 6H), 3.25-3.05 (m, 5H), 2.88 (t, J=2.4 Hz, 1H), 2.65-2.52 (m, 2H), 2.48-2.34 (m, 2H), 1.99 (br s, 4H), 1.47 (s, 9H)); and tert-butyl 4-((1s,3s)-3-(4-(prop-2-yn-1-yloxy)piperidin-1-yl)cyclobutanecarbonyl)piperazine-1-carboxylate (0.5 g, 7% yield) as a white solid (¹H NMR (400 MHz, MeOD) δ 4.29 (t, J=6.8 Hz, 1H), 4.18-4.08 (m, 1H), 3.59-3.53 (m, 2H), 3.48-3.37 (m, 6H), 3.18-3.09 (m, 1H), 2.90-2.80 (m, 1H), 2.56-2.44 (m, 2H), 2.26-2.16 (m, 1H), 2.15-2.03 (m, 1H), 1.47 (s, 9H)).

### Example 1.212. Preparation of 3-{3-methyl-2-oxo-5-[3-({1-[(1R,3R)-3-(piperazine-1-carbonyl)cyclobutyl]piperidin-4-yl}oxy)propyl]-1,3-benzodiazol-1-yl}piperidine-2,6-dione (Intermediate HG)

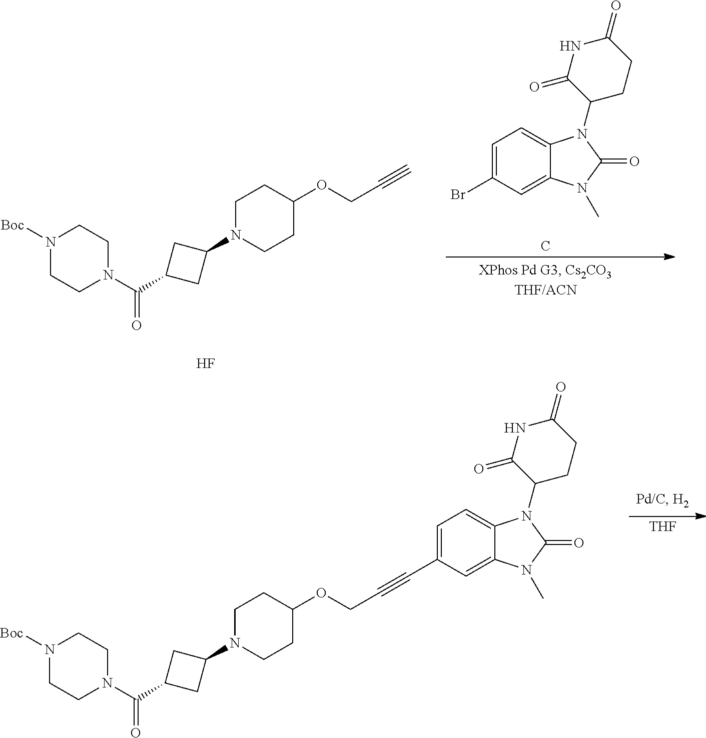

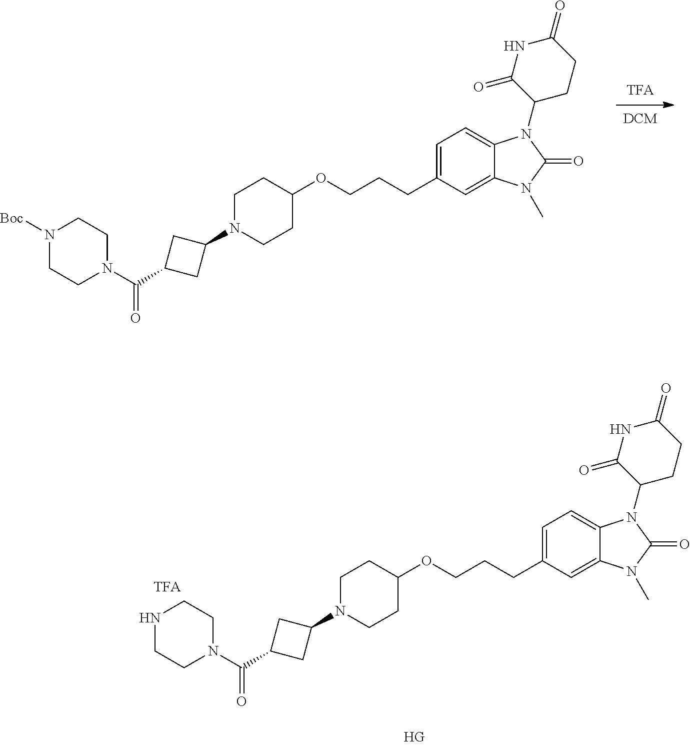 [SMILES: Cn1c(=O)n(C2CCC(=O)NC2=O)c2ccc(CCCOC3CCN([C@H]4C[C@H](C(=O)N5CCN(C(=O)OC(C)(C)C)CC5)C4)CC3)cc21 ; Cn1c(=O)n(C2CCC(=O)NC2=O)c2ccc(CCCOC3CCN([C@H]4C[C@H](C(=O)N5CCNCC5)C4)CC3)cc21]

### Step 1—Tert-butyl 4-((1r,3r)-3-(4-((3-(1-(2,6-dioxopiperidin-3-yl)-3-methyl-2-oxo-2,3-dihydro-1H-benzo[d]imidazol-5-yl)prop-2-yn-1-yl)oxy)piperidin-1-yl)cyclobutanecarbonyl)piperazine-1-carboxylate

To a solution of tert-butyl 4-((1r,3r)-3-(4-(prop-2-yn-1-yloxy)piperidin-1-yl)cyclobutanecarbonyl)piperazine-1-carboxylate (1.83 g, 4.51 mmol, Intermediate HF) and 3-(5-bromo-3-methyl-2-oxo-2,3-dihydro-1H-benzo[d]imidazol-1-yl)piperidine-2,6-dione (1.39 g, 4.10 mmol, Intermediate C) in THF (10 mL) and ACN (10 mL) was added XPhos Pd G3 (174 mg, 205 umol) and Cs₂CO₃ (4.01 g, 12.3 mmol) under N₂. The mixture was stirred at 60° C. for 20 h. On completion, the reaction mixture was quenched with NH₄Cl (15 mL) and extracted with EtOAc (20 mL×2). The combined organic layers were washed with brine (15 mL×3), dried over Na₂SO₄ and evaporated to give the title compound (1.12 g) as orange solid. LC-MS (ESI⁺) m/z 663.4 (M+H)⁺.

### Step 2—Tert-butyl 4-((1r,3r)-3-(4-(3-(1-(2,6-dioxopiperidin-3-yl)-3-methyl-2-oxo-2,3-dihydro-1H-benzo[d]imidazol-5-yl)propoxy)piperidin-1-yl)cyclobutanecarbonyl)piperazine-1-carboxylate

To a solution tert-butyl 4-((1r,3r)-3-(4-((3-(1-(2,6-dioxopiperidin-3-yl)-3-methyl-2-oxo-2,3-dihydro-1H-benzo[d]imidazol-5-yl)prop-2-yn-1-yl)oxy)piperidin-1-yl)cyclobutanecarbonyl)piperazine-1-carboxylate (1.00 g, 1.51 mmol) in THF (2 mL) was added Pd/C (200 mg, 1.51 mmol, 10 wt %) under N₂. The suspension was degassed under vacuum and purged with H₂ several times. The reaction was stirred at 20° C. for 3 hr. On completion, the mixture was filtered and the filtrate was concentrated. The crude product was purified by reversed-phase HPLC (column: YMC Triart C18 250*50 mm*7 um; mobile phase: [water (FA)-ACN]; B %: 10%-40%, 20 min) to afford the title compound (455 mg, 43% yield) as off-white solid. LC-MS (ESI⁺) m/z 667.3 (M+H)⁺; ¹H NMR (400 MHz, DMSO-d₆) δ 11.08 (br s, 1H), 8.17 (s, 1H), 7.04-6.98 (m, 2H), 6.86 (d, J=8 Hz, 1H), 5.33 (dd, J=5.2, 12.8 Hz, 1H), 3.41-3.37 (m, 4H), 3.32 (s, 3H), 3.28 (br d, J=5.6 Hz, 4H), 3.04-2.83 (m, 3H), 2.79-2.70 (m, 2H), 2.68-2.58 (m, 6H), 2.25-2.17 (m, 2H), 2.15-1.87 (m, 6H), 1.85-1.76 (m, 4H), 1.44 (br d, J=9.6 Hz, 2H), 1.40 (s, 9H).

### Step 3—3-{3-methyl-2-oxo-5-[3-({1-[(1R,3R)-3-(piperazine-1-carbonyl)cyclobutyl]piperidin-4-yl}oxy)propyl]-1,3-benzodiazol-1-yl}piperidine-2,6-dione

To a stirred solution of tert-butyl 4-[(1r,3r)-3-(4-{3-[1-(2,6-dioxopiperidin-3-yl)-3-methyl-2-oxo-1,3-benzodiazol-5-yl]propoxy}piperidin-1-yl)cyclobutanecarbonyl]piperazine-1-carboxylate (45 mg, 0.067 mmol) in DCM (1 mL) was added TFA (0.2 mL) dropwise at rt. The resulting mixture was stirred for 1 h at rt under nitrogen atmosphere. On completion, the reaction mixture was concentrated under reduced pressure. The residue was triturated with diethyl ether to afford the title compound (42 mg, 93%) as a yellow solid. LC/MS (ESI, m/z): [(M+1)]⁺=567.3.

### Example 1.213. Preparation of tert-butyl 3-piperazin-1-ylazetidine-1-carboxylate (Intermediate HH)

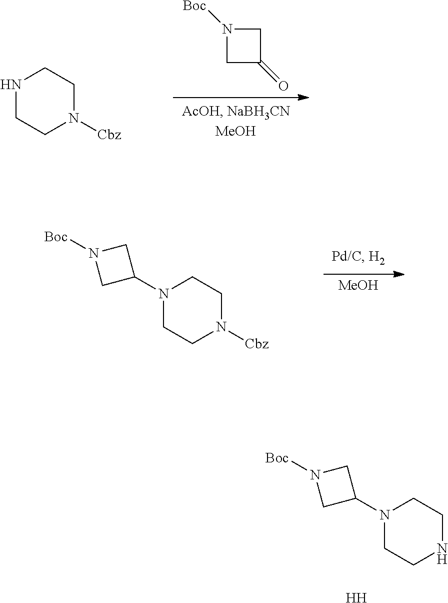

### Step 1—Benzyl 4-(1-tert-butoxycarbonylazetidin-3-yl)piperazine-1-carboxylate

To a solution of benzyl piperazine-1-carboxylate (20 g, 90 mmol, CAS #31166-44-6), tert-butyl 3-oxoazetidine-1-carboxylate (17 g, 100 mmol, CAS #398489-26-4) in methanol (400 mL) was added acetic acid (6.3 g, 105 mmol) and sodium cyanoborohydride (17 g, 270 mmol). The mixture was stirred at 25° C. for 12 hours. On completion, the reaction mixture was quenched with saturated sodium bicarbonate solution (50 mL) at 20° C., and then filtered. The filtrate was concentrated under reduced pressure to give a residue. The residue was purified by column chromatography (SiO₂, Petroleum ether/Ethyl acetate=0/1 to 1/1) to give the title compound (19 g, 51% yield) as a white solid. LC-MS (ESI+) m/z 376.1 (M+H)⁺.

### Step 2—Tert-butyl 3-piperazin-1-ylazetidine-1-carboxylate

Nitrogen was first passed into the reaction vessel, and then Pd/C (2 g, 15.9 mmol, 10 wt %) was added. Then methanol (40 mL) was added by drip to washing vessel wall. Then a solution of benzyl 4-(1-tert-butoxycarbonylazetidin-3-yl) piperazine-1-carboxylate (6 g, 15.9 mmol) in methanol (20 mL) was added to the reaction vessel under nitrogen. The reaction mixture was degassed and purged with hydrogen three times. Then the mixture was stirred at 25° C. for 12 hours under hydrogen. The reaction mixture was filtered and concentrated under reduced pressure to give the title compound (3.5 g) as a white solid. LC-MS (ESI+) m/z 242.2 (M+H)⁺.

### Example 1.214. Preparation of 3-(5-{2-[4-(azetidin-3-yl)piperazin-1-yl]ethyl}-3-methyl-2-oxo-1,3-benzodiazol-1-yl)piperidine-2,6-dione (Intermediate HI)

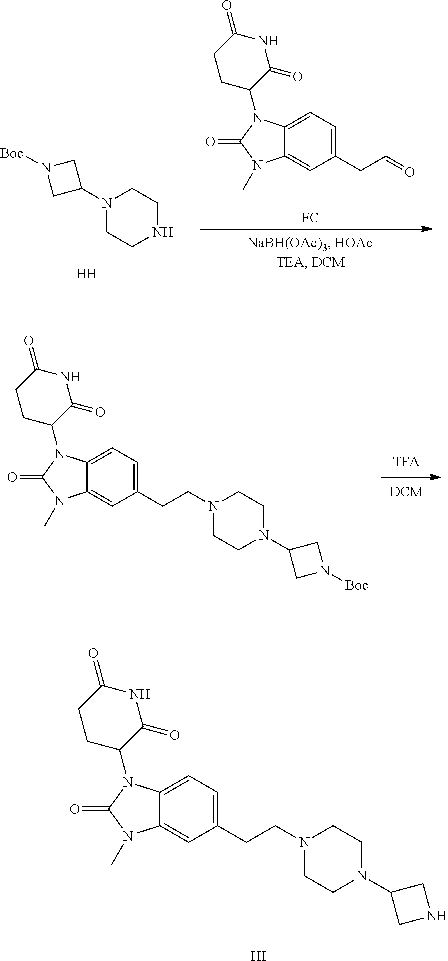

### Step 1—Tert-butyl 3-[4-[2-[1-(2,6-dioxo-3-piperidyl)-3-methyl-2-oxo-benzimidazol-5-yl]ethyl]piperazin-1-yl]azetidine-1-carboxylate

A solution of tert-butyl 3-piperazin-1-ylazetidine-1-carboxylate (2.80 g, 11.6 mmol, Intermediate HH), 2-[1-(2, 6-dioxo-3-piperidyl)-3-methyl-2-oxo-benzimidazol-5-yl]acetaldehyde (3.5 g, 11.6 mmol, Intermediate FC), and triethylamine (1.18 g, 11.6 mmol) in dichloromethane (40 mL) was stirred for 0.5 h at 25° C. Then acetic acid (697 mg, 11.6 mmol) and sodium triacethoxy borohydride (4.92 g, 23.2 mmol) were added to the reaction mixture. The mixture was then stirred at 25° C. for 11.5 hours. On completion, the reaction mixture was diluted with water (50 mL), extracted with ethyl acetate (100 mL×3), dried over anhydrous sodium sulfate, and concentrated under reduced pressure to give a residue. The residue was purified by perp-HPLC (column: Phenomenex Luna C18 150*40 mm*15 um; mobile phase: [water (FA)-ACN]; B %: 10%-20%, 10 min) to give the title compound (982 mg, 14% yield) as a white solid. ¹H NMR (400 MHz, DMSO-d₆) δ=11.08 (s, 1H), 7.06 (s, 1H), 6.99 (d, J=8.0 Hz, 1H), 6.88 (m, 1H), 5.35-5.30 (m, 1H), 3.81 (s, 4H), 3.64 (s, 4H), 3.31 (s, 3H), 3.05-2.84 (m, 4H), 2.77-2.63 (m, 4H), 2.33-2.21 (m, 4H), 1.98 (s, 1H), 1.37 (s, 9H); LC-MS (ESI+) m/z 527.3 (M+H)⁺.

### Step 2—3-(5-{2-[4-(Azetidin-3-yl)piperazin-1-yl]ethyl}-3-methyl-2-oxo-1,3-benzodiazol-1-yl)piperidine-2,6-dione Trifluoroacetate

To a stirred solution of tert-butyl 3-(4-{2-[1-(2,6-dioxopiperidin-3-yl)-3-methyl-2-oxo-1,3-benzodiazol-5-yl]ethyl}piperazin-1-yl)azetidine-1-carboxylate (45 mg, 0.085 mmol) in DCM (1 mL) was added TFA (0.2 mL) dropwise at rt under air atmosphere. The resulting mixture was stirred for 1 h at rt. On completion, the reaction mixture was concentrated under reduced pressure. The residue was triturated with diethyl ether to afford the title compound (65 mg) as a white solid. LC/MS (ESI, m/z): [(M+1)]⁺=427.3.

### Example 1.215. Preparation of tert-butyl N-[4-[[(3R)-3-piperazin-1-ylpyrrolidin-1-yl]methyl]cyclohexyl]carbamate (Intermediate HJ)

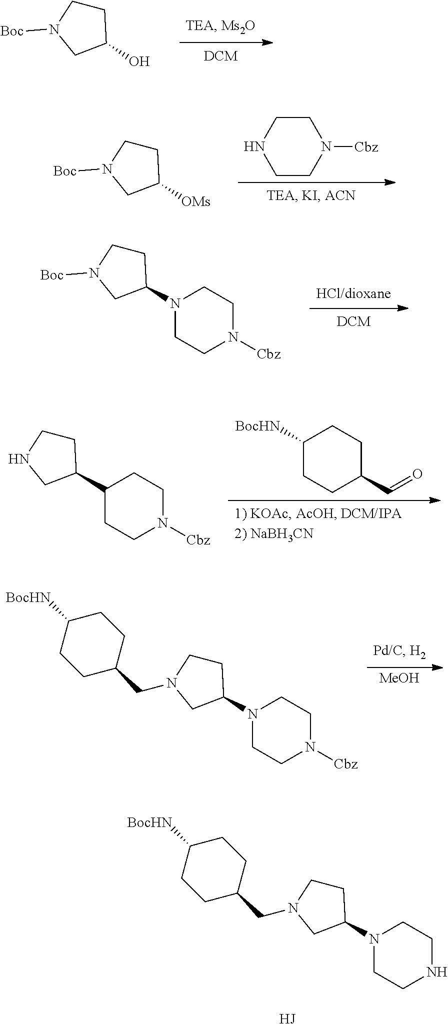

### Step 1—Tert-butyl (3S)-3-methylsulfonyloxypyrrolidine-1-carboxylate

To a solution of tert-butyl (3S)-3-hydroxypyrrolidine-1-carboxylate (24 g, 128 mmol, CAS #101469-92-5) in dichloromethane (300 mL) at 0° C. was added triethylamine (38.9 g, 384 mmol), and methylsulfonyl methanesulfonate (66.9 g, 384 mmol). The reaction mixture was stirred at 0° C. for 1 hr, then stirred at 25° C. for 2 h. The reaction mixture was quenched with water (200 mL) at 0° C., and extracted with dichloromethane (100 mL×3), dried over anhydrous sodium sulfate, and concentrated under reduced pressure to give the title compound (25 g) as a white solid. LC-MS (ESI+) m/z 210.4 (M−55)⁺.

### Step 2—Benzyl 4-[(3R)-1-tert-butoxycarbonylpyrrolidin-3-yl]piperazine-1-carboxylate

A mixture of tert-butyl (3 S)-3-methylsulfonyloxypyrrolidine-1-carboxylate (24.5 g, 92.3 mmol), benzyl piperazine-1-carboxylate (40.6 g, 184 mmol), triethylamine (28.0 g, 277 mmol; CAS #31166-44-6), and potassium iodide (22.9 g, 138 mmol) in acetonitrile (300 mL) was degassed and purged with nitrogen three times, and then the mixture was stirred at 80° C. for 12 hours under nitrogen atmosphere. On completion, the reaction mixture was concentrated under reduced pressure to give a residue, then it was diluted with water (100 mL), and extracted with ethyl acetate (100 mL×3). The combined organic layer was dried over anhydrous sodium sulfate, and concentrated under reduced pressure to give a residue. The crude was purified by silica gel column chromatography (Petroleum ether/Ethyl acetate=1:1) to give the title compound (16 g, 44% yield) as a white solid. LC-MS (ESI+) m/z 390.2 (M+H)⁺.

### Step 3—Benzyl 4-[(3R)-pyrrolidin-3-yl]piperazine-1-carboxylate

To a solution of benzyl 4-[(3R)-1-tert-butoxycarbonylpyrrolidin-3-yl]piperazine-1-carboxylate (15.5 g, 39.8 mmol) in dichloromethane (100 mL) was added hydrochloric acid/dioxane (500 mL). The mixture was stirred at 25° C. for 3 hours. On completion, the reaction mixture was concentrated under reduced pressure to give the title compound (11 g, HCl) as a white solid. LC-MS (ESI+) m/z 290.2 (M+H)⁺.

### Step 4—Benzyl 4-[(3R)-1-[[4-(tert-butoxycarbonylamino)cyclohexyl]methyl]pyrrolidin-3-yl]piperazine-1-carboxylate

To a solution of benzyl 4-[(3R)-pyrrolidin-3-yl]piperazine-1-carboxylate (10 g, 30.6 mmol) in dichloromethane (150 mL) and isopropyl alcohol (100 mL) was added potassium acetate (4.52 g, 46.0 mmol). After stirring at 25° C. for 1 hr, acetic acid (5.53 g, 92.0 mmol) and tert-butyl N-(4-formylcyclohexyl)carbamate (9.07 g, 39.9 mmol, CAS #181308-57-6) was added. After stirring at 25° C. for 4 hours, sodium cyanoborohydride (5.79 g, 92.0 mmol) was added to the reaction mixture and the mixture was stirred at 25° C. for 7 hours. On completion, the reaction mixture was quenched with water (100 mL) at 0° C., and extracted with ethyl acetate (30 mL×3). The combined organic layer was dried over anhydrous sodium sulfate, and concentrated under reduced pressure to give a residue. The residue was purified by column chromatography (SiO₂, Petroleum ether/Ethyl acetate=1:1-0:1) to give the title compound (10 g, 65% yield) as a white solid. LC-MS (ESI+) m/z 501.4 (M+H)⁺.

### Step 5—tert-butyl N-[4-[[(3R)-3-piperazin-1-ylpyrrolidin-1-yl]methyl]cyclohexyl]carbamate

Nitrogen was first passed into the reaction vessel, and then Pd/C (2 g, 8 mmol, 10 wt %) was added. Next, methanol (40 mL) was added by drip washing the vessel wall. Then a solution of benzyl 4-[(3R)-1-[[4-(tert-butoxycarbonylamino) cyclohexyl]methyl]pyrrolidin-3-yl]piperazine-1-carboxylate (4 g, 7.99 mmol) in methanol (40 mL) was added to the reaction vessel under nitrogen. The mixture was degassed and purged with hydrogen three times, then the mixture was stirred at 25° C. for 12 hours under hydrogen at 15 psi. The reaction mixture was filtered and concentrated under reduced pressure to give the title compound (2 g) as a white solid. LC-MS (ESI+) m/z 367.1 (M+H)⁺.

### Example 1.216. Preparation of 3-[3-methyl-2-oxo-5-({4-[(3R)-1-{[(1r,4r)-4-aminocyclohexyl]methyl}pyrrolidin-3-yl]piperazin-1-yl}methyl)-1,3-benzodiazol-1-yl]piperidine-2,6-dione (Intermediate HK)

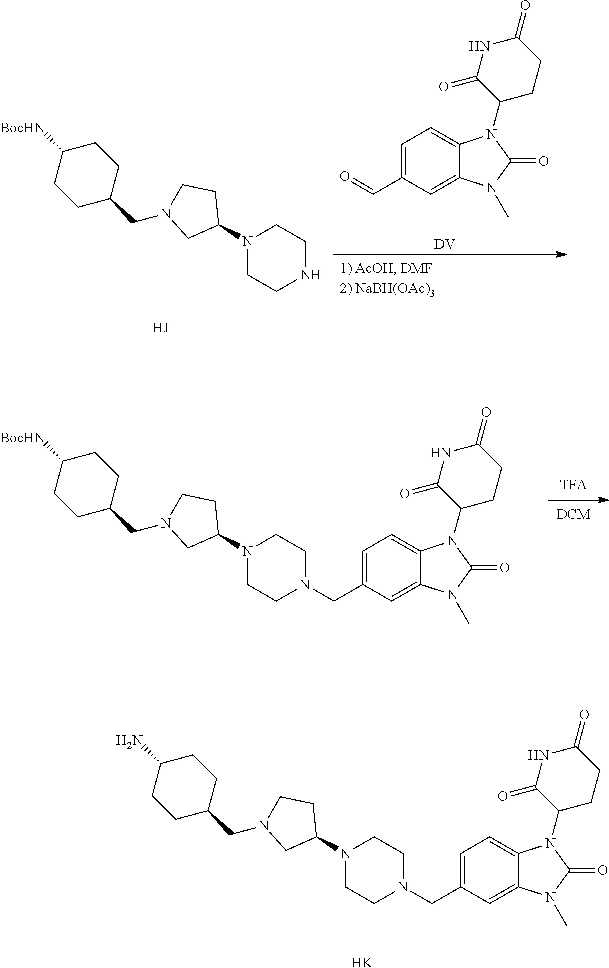

### Step 1—tert-butyl N-[4-[[(3R)-3-[4-[[1-(2,6-dioxo-3-piperidyl)-3-methyl-2-oxo-benzimidazol-5-yl]methyl]piperazin-1-yl]pyrrolidin-1-yl]methyl]cyclohexyl]carbamate

To a solution of tert-butyl N-[4-[[(3R)-3-piperazin-1-ylpyrrolidin-1-yl]methyl]cyclohexyl]carbamate (3.9 g, 10.6 mmol, Intermediate HJ) in dimethylformamide (20 mL) was added acetic acid (1.92 g, 31.9 mmol) and 1-(2,6-dioxo-3-piperidyl)-3-methyl-2-oxo-benzimidazole-5-carbaldehyde (3.97 g, 13.8 mmol, Intermediate DV). After stirring at 25° C. for 1 hr, sodium triacethoxy borohydride (6.77 g, 31.9 mmol) was added to the reaction mixture and stirred at 25° C. for 11 hrs. On completion, the reaction mixture was quenched with water (100 mL) at 0° C., and extracted with ethyl acetate 90 mL (30 mL×3). The combined organic layers were dried over anhydrous sodium sulfate, and a residue was obtained after filtration and concentration. The residue was purified by perp-HPLC (column: Phenomenex Luna C18; 150×40 mm×15 um; mobile phase: [water (TFA)-ACN]; B %: 3%-33%, 10 min) to give the title compound (1.68 g, 20% yield) as a white solid. ¹H NMR (400 MHz, DMSO-d₆) δ=11.12 (s, 1H), 10.08-9.78 (m, 1H), 7.26-7.14 (m, 2H), 6.76 (d, J=7.6 Hz, 1H), 5.44-5.39 (m, 1H), 4.35 (s, 2H), 3.72-3.55 (m, 1H), 3.35 (s, 3H), 3.20-3.11 (m, 2H), 3.08-2.90 (m, 8H), 2.79-2.58 (m, 2H), 2.45-2.28 (m, 2H), 2.08-1.93 (m, 2H), 1.75 (s, 4H), 1.62-1.42 (m, 3H), 1.38 (s, 3H), 1.37 (s, 9H), 1.19-1.07 (m, 2H), 0.97 (d, J=11.6 Hz, 2H); LC-MS (ESI+) m/z 638.7 (M+H)⁺.

### Step 2—3-[3-Methyl-2-oxo-5-({4-[(3R)-1-{[(1r,4r)-4-aminocyclohexyl]methyl}pyrrolidin-3-yl]piperazin-1-yl}methyl)-1,3-benzodiazol-1-yl]piperidine-2,6-dione Trifluoroacetate

To a stirred solution of tert-butyl N-[(1R,4R)-4-{[(3R)-3-(4-{[1-(2,6-dioxopiperidin-3-yl)-3-methyl-2-oxo-1,3-benzodiazol-5-yl]methyl}piperazin-1-yl)pyrrolidin-1-yl]methyl}cyclohexyl]carbamate (45 mg, 0.071 mmol) in DCM (1 mL) was added TFA (0.2 mL) dropwise at rt. The resulting mixture was stirred for 1 h at rt under nitrogen atmosphere. On completion, the reaction mixture was concentrated under reduced pressure. The residue was purified by triturated with diethyl ether to afford the title compound (56 mg) as a white solid. LC/MS (ESI, m/z): [(M+1)]⁺=538.3.

### Example 1.217. Preparation of 3-[3-Methyl-4-[[4-(methylamino)-1-piperidyl]methyl]-2-oxo-benzimidazol-1-yl]piperidine-2,6-dione (Intermediate HL)

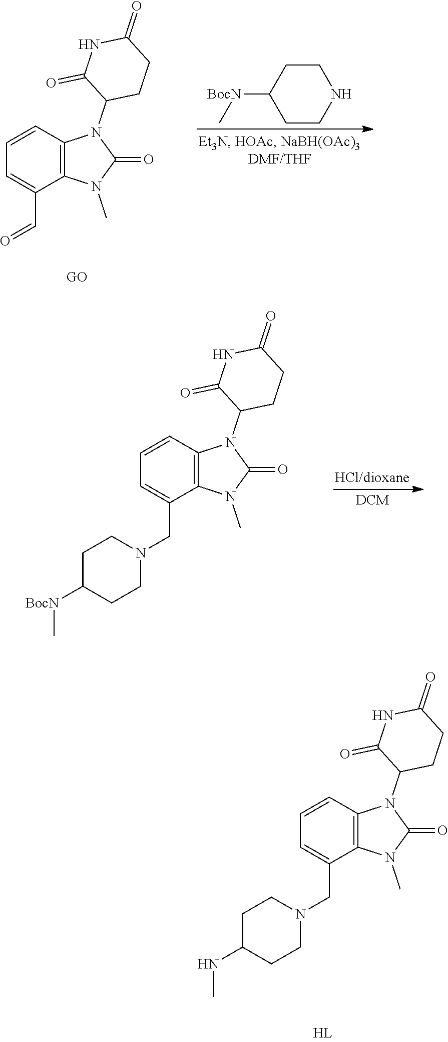 [SMILES: Cn1c(=O)n(C2CCC(=O)NC2=O)c2cccc(C=O)c21 ; CN(C(=O)OC(C)(C)C)C1CCNCC1 ; CN(C(=O)OC(C)(C)C)C1CCN(Cc2cccc3c2n(C)c(=O)n3C2CCC(=O)NC2=O)CC1 ; CNC1CCN(Cc2cccc3c2n(C)c(=O)n3C2CCC(=O)NC2=O)CC1]

### Step 1—Tert-butyl N-[1-[[1-(2,6-dioxo-3-piperidyl)-3-methyl-2-oxo-benzimidazol-4-yl]methyl]-4-piperidyl]-N-methyl-carbamate

To a mixture of 1-(2,6-dioxo-3-piperidyl)-3-methyl-2-oxo-benzimidazole-4-carbaldehyde (7.00 g, 12.1 mmol, Intermediate GO) and tert-butyl N-methyl-N-(4-piperidyl)carbamate (3.92 g, 18.2 mmol, CAS #108612-54-0) in tetrahydrofuran (70 mL) and dimethylformamide (70 mL) was added triethylamine (1.23 g, 12.2 mmol), acetic acid (731 mg, 12.1 mmol) and sodium triacethoxy borohydride (3.87 g, 18.2 mmol) at 0° C., then the reaction mixture was stirred at 25° C. for 12 hrs. On completion, the reaction mixture was diluted with water (100 mL) and extracted with ethyl acetate (100 mL×2). The combined organic layers were washed with brine (80 mL×2), dried over anhydrous sodium sulfate, filtered and concentrated under reduced pressure to give a residue. The residue was purified by reversed-phase HPLC (0.1% FA condition) to give the title compound (1.5 g, 25% yield) as white solid. LC-MS (ESI+) m/z 486.3 (M+H)⁺.

### Step 2—3-[3-methyl-4-[[4-(methylamino)-1-piperidyl]methyl]-2-oxo-benzimidazol-1-yl]piperidine-2,6-dione

To a mixture of tert-butyl N-[1-[[1-(2,6-dioxo-3-piperidyl)-3-methyl-2-oxo-benzimidazol-4-yl]methyl]-4-piperidyl]-N-methyl-carbamate (1.35 g, 2.78 mmol) in dichloromethane (15 mL) was added hydrochloric acid/dioxane (4 M, 3 mL), then the reaction mixture was stirred at 25° C. for 1 hour. The reaction mixture was concentrated in vacuo to give the title compound (1.1 g, HCl salt) as white solid. LC-MS (ESI+) m/z 386.3 (M+H)⁺.

### Example 1.218. Preparation of 3-[3-methyl-4-({4-[methyl({[(1r,4r)-4-aminocyclohexyl]methyl})amino]piperidin-1-yl}methyl)-2-oxo-1,3-benzodiazol-1-yl]piperidine-2,6-dione (Intermediate HM)

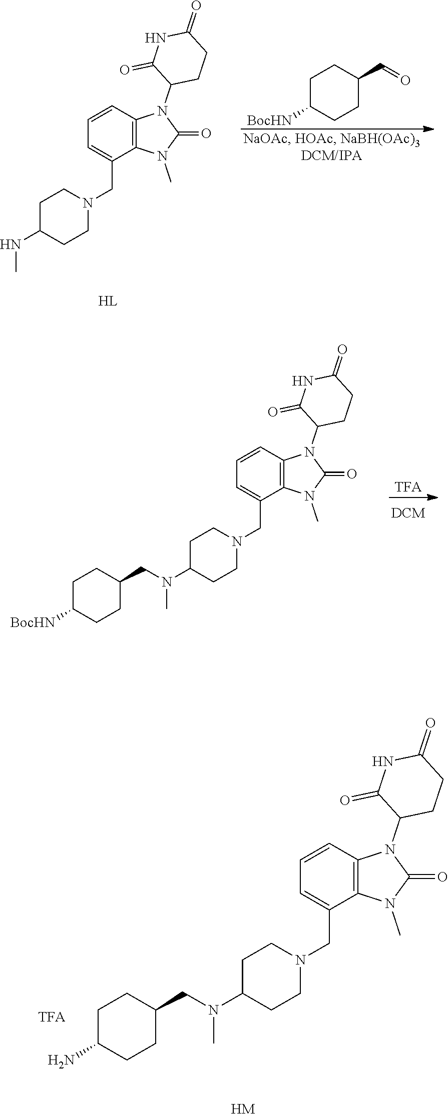

### Step 1—Tert-butyl N-[4-[[[1-[[1-(2,6-dioxo-3-piperidyl)-3-methyl-2-oxo-benzimidazol-4-yl]methyl]-4-piperidyl]-methyl-amino]methyl]cyclohexyl]carbamate

To a mixture of 3-[3-methyl-4-[[4-(methylamino)-1-piperidyl]methyl]-2-oxo-benzimidazol-1-yl]piperidine-2,6-dione (1.10 g, 2.61 mmol, Intermediate HL) in dichloromethane (5 mL) and isopropyl alcohol (10 mL) was added sodium acetate (1.07 g, 13.0 mmol) at 25° C. and stirred for 30 minutes. Then to the mixture was added tert-butyl N-(4-formylcyclohexyl)carbamate (888 mg, 3.91 mmol) and acetic acid (313 mg, 5.21 mmol) at 25° C. and the mixture was stirred for another 30 minutes. To the resulting mixture was added sodium triacethoxy borohydride (1.11 g, 5.21 mmol) at 0° C. and then stirred at 25° C. for 11 hours. The reaction mixture was diluted with water (40 ml) and extracted with ethyl acetate (40 mL×2). The combined organic layers were washed with brine (40 mL×2), dried over anhydrous sodium sulfate, filtered and concentrated under reduced pressure to give a residue. The residue was purified by reversed-phase HPLC (0.1% FA condition) to give the title compound (1.10 g) as a white solid. ¹H NMR (400 MHz, DMSO-d₆) δ=11.10 (s, 1H), 8.23 (s, 1H), 7.20-7.10 (m, 1H), 7.05-6.95 (m, 1H), 6.90-6.85 (m, 1H), 6.75-6.60 (m, 1H), 5.50-5.30 (m, 1H), 3.70 (s, 3H), 3.65 (s, 2H), 3.05-2.85 (m, 2H), 2.75-2.60 (m, 2H), 2.45-2.40 (m, 2H), 2.30 (s, 3H), 2.30-2.15 (m, 1H), 2.10-1.90 (m, 2H), 1.80-1.65 (m, 6H), 1.55-1.47 (m, 2H), 1.45 (s, 12H), 1.20-1.05 (m, 2H), 0.90-0.70 (m, 2H); LC-MS (ESI+) m/z 597.6 (M+1)⁺.

### Step 2—3-[3-Methyl-4-({4-[methyl({[(1r,4r)-4-aminocyclohexyl]methyl})amino]piperidin-1-yl}methyl)-2-oxo-1,3-benzodiazol-1-yl]piperidine-2,6-dione trifluoroacetate

To a stirred solution of tert-butyl N-[(1r,4r)-4-{[(1-{[1-(2,6-dioxopiperidin-3-yl)-3-methyl-2-oxo-1,3-benzodiazol-4-yl]methyl}piperidin-4-yl)(methyl)amino]methyl}cyclohexyl]carbamate (45 mg, 0.075 mmol) in DCM (1 mL) was added TFA (0.2 mL) dropwise at rt. The resulting mixture was stirred for 1 h at rt. On completion, the reaction mixture was concentrated under reduced pressure. The residue was triturated with diethyl ether to afford the title compound (37 mg) as an off-white solid. LC/MS (ESI, m/z): [(M+1)]⁺=497.3.

### Example 1.219. Preparation of 3-[4-(2,7-diazaspiro[3.5]nonan-2-ylmethyl)-3-methyl-2-oxo-benzimidazol-1-yl]piperidine-2,6-dione (Intermediate HN)

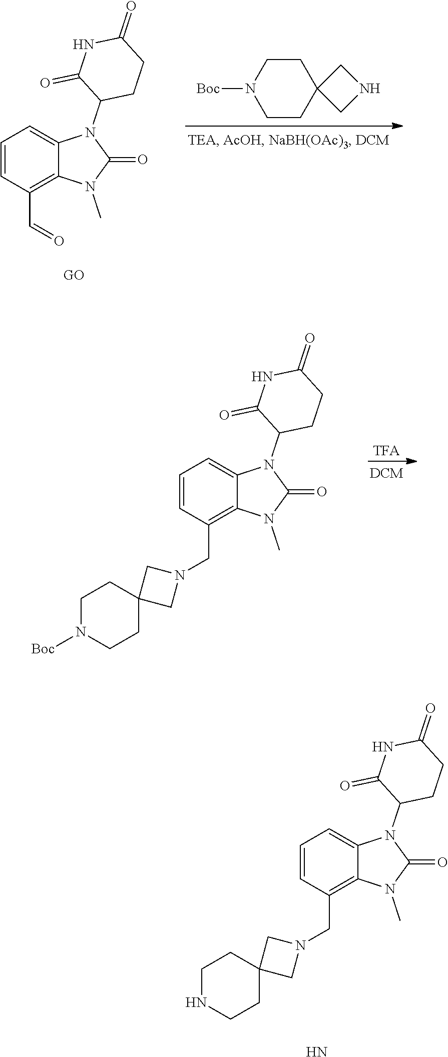

### Step 1—Tert-butyl 2-[[1-(2,6-dioxo-3-piperidyl)-3-methyl-2-oxo-benzimidazol-4-yl]methyl]-2,7-diazaspiro[3.5]nonane-7-carboxylate

To a solution of 1-(2,6-dioxo-3-piperidyl)-3-methyl-2-oxo-benzimidazole-4-carbaldehyde (3 g, 10.44 mmol, Intermediate GO) and tert-butyl 2,7-diazaspiro[3.5]nonane-7-carboxylate (3.55 g, 15.66 mmol, CAS #896464-16-7) in DCM (100 mL) was added KOAc (5.12 g, 52.2 mmol). The mixture was stirred at 25° C. for 1 hr. Then NaBH(OAc)₃ (6.64 g, 31.3 mmol) was added to the mixture under N₂ and it was stirred at 25° C. for 5 hr. On completion, the reaction was quenched with water (100 mL), and extracted with EA (3×100 mL). The combined organic layer was concentrated in vacuo to give a residue. The residue was purified by reversed-phase HPLC (0.1% FA condition) to give the title compound (2.8 g, 54% yield) as yellow solid. LC-MS (ESI+) m/z 498.2 (M+H)⁺.

### Step 2—3-[4-(2,7-Diazaspiro[3.5]nonan-2-ylmethyl)-3-methyl-2-oxo-benzimidazol-1-yl]piperidine-2,6-dione

To a solution of tert-butyl 2-[[1-(2,6-dioxo-3-piperidyl)-3-methyl-2-oxo-benzimidazol-4-yl]methyl]-2,7-diazaspiro[3.5]nonane-7-carboxylate (1.9 g, 3.82 mmol) in DCM (50 mL) was added TFA (7.70 g, 67.53 mmol, 5 mL). The mixture was stirred at 25° C. for 0.5 hr. On completion, the reaction mixture was concentrated in vacuo to give the title compound (1.52 g) as yellow oil. LC-MS (ESI⁺) m/z 398.0 (M+H)⁺.

### Example 1.220. Preparation of 3-{3-Methyl-2-oxo-4-[(7-{[(1r,4r)-4-aminocyclohexyl]methyl}-2,7-diazaspiro[3.5]nonan-2-yl)methyl]-1,3-benzodiazol-1-yl}piperidine-2,6-dione (Intermediate HO)

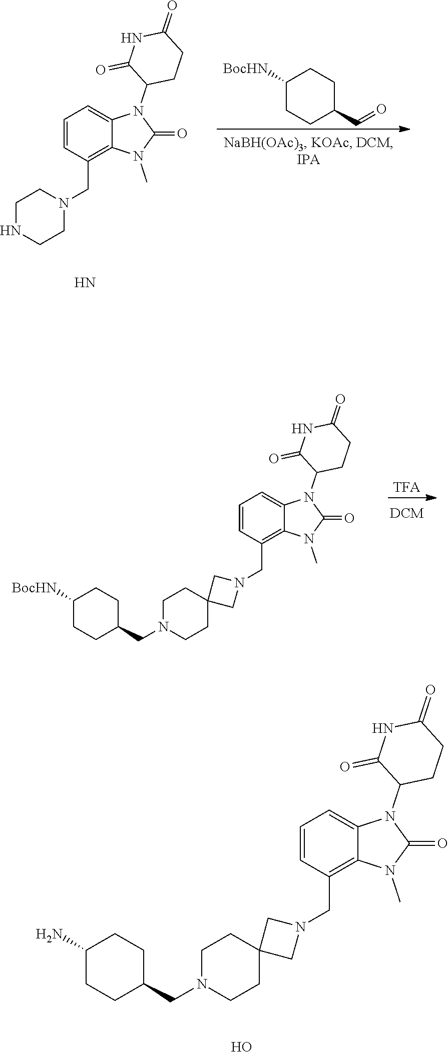

### Step 1—3-[4-(2,7-Diazaspiro[3.5]nonan-2-ylmethyl)-3-methyl-2-oxo-benzimidazol-1-yl]piperidine-2,6-dione

To a solution of 3-[4-(2,7-diazaspiro[3.5]nonan-2-ylmethyl)-3-methyl-2-oxo-benzimidazol-1-yl]piperidine-2,6-dione (1.52 g, 3.82 mmol, Intermediate HN) and tert-butyl N-(4-formylcyclohexyl)carbamate (1.30 g, 5.74 mmol, CAS #181308-57-6) in DCM (50 mL) and IPA (50 mL) was added KOAc (1.88 g, 19.1 mmol). The mixture was stirred at 25° C. for 1 hr. Then NaBH(OAc)₃ (2.43 g, 11.5 mmol) was added to the mixture and it was stirred at 25° C. for 5 hrs. On completion, the reaction was filtered and the filtrate was concentrated in vacuo. The crude product was purified by reversed-phase HPLC (0.1% FA condition) to give the title compound (2.5 g, 95% yield, FA) as white solid. ¹H NMR (400 MHz, DMSO-d6) δ=11.10 (s, 1H), 7.09 (s, 1H), 7.00-6.92 (m, 2H), 6.75 (d, J=8.0 Hz, 1H), 5.41-5.36 (m, 1H), 3.63 (s, 3H), 3.18-3.11 (m, 6H), 2.94-2.82 (m, 2H), 2.79-2.56 (m, 5H), 2.01-1.84 (m, 5H), 1.80-1.75 (m, 6H), 1.37 (s, 11H), 1.15-1.08 (m, 2H), 0.99-0.87 (m, 2H). LC-MS (ESI⁺) m/z 609.2 (M+H)⁺.

### Step 2—3-{3-Methyl-2-oxo-4-[(7-{[(1r,4r)-4-aminocyclohexyl]methyl}-2,7-diazaspiro[3.5]nonan-2-yl)methyl]-1,3-benzodiazol-1-yl}piperidine-2,6-dione Trifluoroacetate

To a stirred solution of tert-butyl N-[(1r,4r)-4-[(2-{[1-(2,6-dioxopiperidin-3-yl)-3-methyl-2-oxo-1,3-benzodiazol-4-yl]methyl}-2,7-diazaspiro[3.5]nonan-7-yl)methyl]cyclohexyl]carbamate (45 mg, 0.074 mmol) in DCM (1 mL) was added TFA (0.2 mL) dropwise at rt. The resulting mixture was stirred for 1 h at rt. On completion, the reaction mixture was concentrated under reduced pressure. The residue was triturated with diethyl ether to afford the title compound (40 mg) as a white solid. LC/MS (ESI, m/z): [(M+1)]=509.4.

### Example 1.221. Preparation of 3-[3-Methyl-2-oxo-4-(4-piperidyl)benzimidazol-1-yl]piperidine-2,6-dione (Intermediate HP)

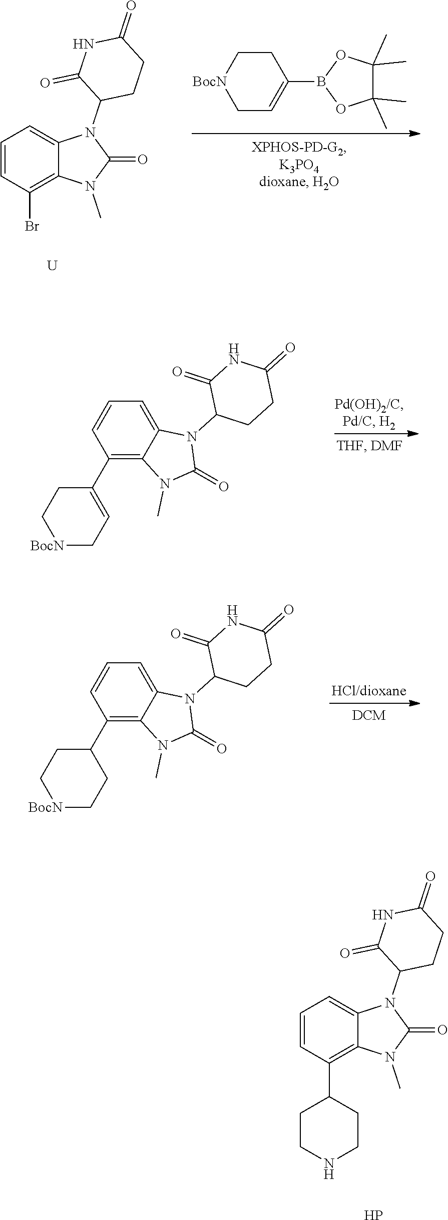

### Step 1—Tert-butyl 4-[1-(2,6-dioxo-3-piperidyl)-3-methyl-2-oxo-benzimidazol-4-yl]-3,6-dihydro-2H-pyridine-1-carboxylate

A mixture of 3-(4-bromo-3-methyl-2-oxo-benzimidazol-1-yl)piperidine-2,6-dione (15 g, 44.4 mmol, Intermediate U), tert-butyl 4-(4,4,5,5-tetramethyl-1,3,2-dioxaborolan-2-yl)-3,6-dihydro-2H-pyridine-1-carboxylate (CAS #286961-14-6, 20.6 g, 66.5 mmol), chloro(2-dicyclohexylphosphino-2′,4′,6′-triisopropyl-1,1′-biphenyl)[2-(2′-amino-1,1′-biphenyl)]palladium(II) (3.49 g, 4.44 mmol) in dioxane (250 mL) and water (25 mL) was added potassium phosphate (18.8 g, 88.7 mmol). The mixture was degassed and purged with nitrogen three times, and then was stirred at 80° C. for 4 hours under nitrogen atmosphere. The reaction mixture was quenched with ammonium chloride (50 mL) at 25° C., and then diluted with water (20 mL) and extracted with ethyl acetate (200 mL×3). The combined organic layers were washed with brine (100 mL×3), dried over anhydrous sodium sulfate, filtered and concentrated under reduced pressure to give a residue. The residue was triturated with ethyl acetate (50 mL) at 25° C. for 30 minutes and then filtered. The filter cake was dried in vacuo to give the title compound (17.7 g, 89% yield) as white solid. ¹H NMR (400 MHz, DMSO-d6) δ=11.12 (s, 1H), 7.12-6.96 (m, 2H), 6.84 (dd, J=7.6, 1.2 Hz, 1H), 5.72 (s, 1H), 5.42-5.37 (m, 1H), 4.08-3.96 (m, 2H), 3.60 (s, 2H), 3.32 (s, 3H), 2.96-2.84 (m, 1H), 2.78-2.60 (m, 2H), 2.40 (s, 2H), 2.08-1.96 (m, 1H), 1.44 (s, 9H).

### Step 2—Tert-butyl 4-[1-(2,6-dioxo-3-piperidyl)-3-methyl-2-oxo-benzimidazol-4-yl]piperidine-1-carboxylate

To a suspension of Pd/C (2.8 g, 10 wt %) and Pd(OH)2/C (2.8 g, 4.0 mmol, 20 wt %) in tetrahydrofuran (160 mL) was added a solution of tert-butyl 4-[1-(2,6-dioxo-3-piperidyl)-3-methyl-2-oxo-benzimidazol-4-yl]-3,6-dihydro-2H-pyridine-1-carboxylate (8 g, 20 mmol) in dimethylformamide (320 mL) and tetrahydrofuran (160 mL) under nitrogen. The mixture was degassed and purged with hydrogen three times at 25° C., Then the mixture was stirred at 60° C. for 12 hours under hydrogen at 15 psi. The reaction mixture was filtered, washed with THF (600 mL) and the filtrate was concentrated under reduced pressure to give a residue. The residue was triturated with ethyl acetate (50 mL) at 25° C. for 30 minutes and filtered. The filter cake was dried in vacuo to give the title compound (3.9 g, 48% yield) as white solid. ¹H NMR (400 MHz, DMSO-d6) δ=11.11 (s, 1H), 7.08-6.92 (m, 3H), 5.41-5.36 (m, 1H), 4.08 (d, J=9.2 Hz, 2H), 3.60 (s, 3H), 3.48-3.38 (m, 1H), 3.00-2.80 (m, 3H), 2.72-2.60 (m, 2H), 2.04-1.96 (m, 1H), 1.84 (d, J=12.4 Hz, 2H), 1.64-1.52 (m, 2H), 1.44 (s, 9H); LC-MS (ESI⁺) m/z 465.3 (M+Na)⁺.

### Step 3—3-[3-methyl-2-oxo-4-(4-piperidyl)benzimidazol-1-yl]piperidine-2,6-

To a solution of tert-butyl 4-[1-(2,6-dioxo-3-piperidyl)-3-methyl-2-oxo-benzimidazol-4-yl]piperidine-1-carboxylate (5.3 g, 12 mmol) in dichloromethane (50 mL) was added hydrochloric acid/dioxane (4 M, 2.99 mL). The mixture was stirred at 25° C. for 0.5 hour and then concentrated under reduced pressure to give the title compound (4.7 g, HCl salt) as white solid. LC-MS (ESI+) m/z 343.2 (M+H)⁺.

### Example 1.222. Preparation of 3-{4-[1-(Azetidin-3-ylmethyl)piperidin-4-yl]-3-methyl-2-oxo-1,3-benzodiazol-1-yl}piperidine-2,6-dione (Intermediate HQ)

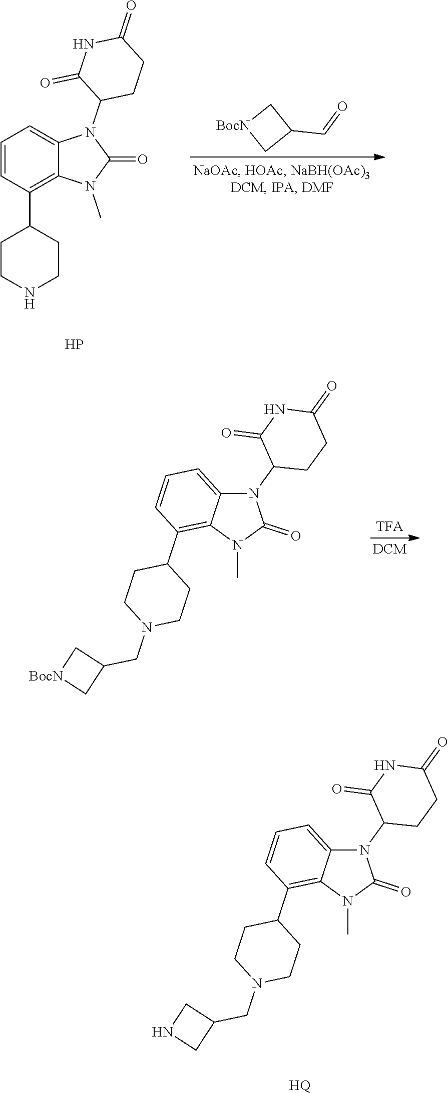

### Step 1—Tert-butyl 3-[[4-[1-(2,6-dioxo-3-piperidyl)-3-methyl-2-oxo-benzimidazol-4-yl]-1-piperidyl]methyl]azetidine-1-carboxylate

To a solution of 3-[3-methyl-2-oxo-4-(4-piperidyl)benzimidazol-1-yl]piperidine-2,6-dione (8 g, 21.1 mmol, HCl, Intermediate HP) in dichloromethane (40 mL), isopropyl alcohol (80 mL) and dimethylformamide (40 mL) was added sodium acetate (4.33 g, 52.8 mmol) at 25° C. and the mixture was stirred for 30 minutes. Then to the mixture was added tert-butyl 3-formylazetidine-1-carboxylate (3.26 g, 17.6 mmol, CAS #177947-96-5) in acetic acid (2.11 g, 35.2 mmol) at 25° C. and stirred for another 30 minutes. Sodium triacethoxy borohydride (7.46 g, 35.2 mmol) was then added to the mixture at 0° C. and the mixture was stirred at 25° C. for 11 hrs. The reaction mixture was quenched with 1N aqueous sodium bicarbonate until the pH=7, then diluted with water (300 mL) and extracted with ethyl acetate (200 mL×4). The combined organic layers were washed with brine (200 mL×3), dried over anhydrous sodium sulfate, filtered and concentrated under reduced pressure to give a residue. The residue was triturated with ethyl acetate (30 mL) at 25° C. for 30 minutes and filtered. The filter cake was dried in vacuo to give the title compound (4.87 g, 54% yield) as a white solid. ¹H NMR (400 MHz, DMSO-d6) δ=11.08 (s, 1H), 6.92-7.08 (m, 3H), 5.40-5.36 (m, 1H), 3.92 (s, 2H), 3.58 (s, 3H), 3.50 (s, 2H), 3.20 (t, J=11.2 Hz, 1H), 2.96-2.84 (m, 3H), 2.80-2.62 (m, 3H), 2.58-2.55 (m, 2H), 2.10 (t, J=10.4 Hz, 2H), 2.04-1.96 (m, 1H), 1.85-1.65 (m, 4H), 1.38 (s, 9H); LC-MS (ESI+) m/z 512.4 (M+H)⁺.

### Step 2—3-{4-[1-(Azetidin-3-ylmethyl)piperidin-4-yl]-3-methyl-2-oxo-1,3-benzodiazol-1-yl}piperidine-2,6-dione Trifluoroacetate

To a stirred solution of tert-butyl 3-({4-[1-(2,6-dioxopiperidin-3-yl)-3-methyl-2-oxo-1,3-benzodiazol-4-yl]piperidin-1-yl}methyl)azetidine-1-carboxylate (45 mg, 0.088 mmol) in DCM (1 mL) was added TFA (0.2 mL) dropwise at rt. The resulting mixture was stirred for 1 h at rt. On completion, the reaction mixture was concentrated under reduced pressure. The residue was triturated with diethyl ether to afford the title compound (40 mg) as a white solid. LC/MS (ESI, m/z): [(M+1)]⁺=412.2.

### Example 1.223. Preparation of 6-[(5-bromopyridin-2-yl)amino]-4-{[5-(5-fluoropyrimidin-2-yl)-4-methoxypyridin-3-yl]amino}-N-methylpyridine-3-carboxamide (Intermediate HR)

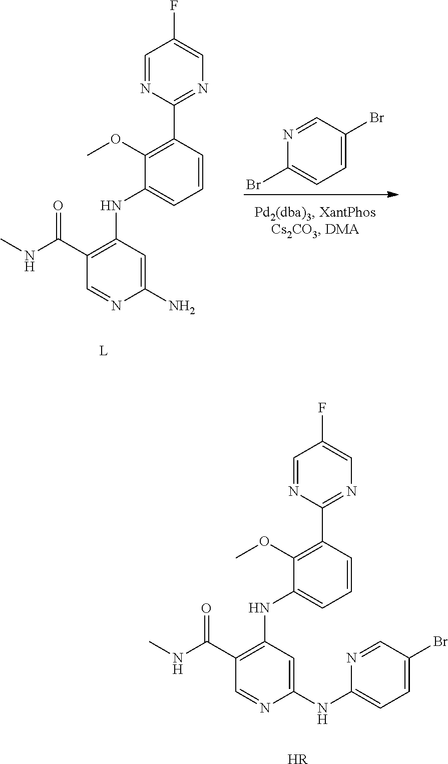 [SMILES: CNC(=O)c1cnc(N)cc1Nc1cccc(-c2ncc(F)cn2)c1OC ; Brc1ccc(Br)nc1 ; CNC(=O)c1cnc(Nc2ccc(Br)cn2)cc1Nc1cccc(-c2ncc(F)cn2)c1OC ; "HR"]

To a stirred mixture of 6-amino-4-{[3-(5-fluoropyrimidin-2-yl)-2-methoxyphenyl]amino}-N-methylpyridine-3-carboxamide (1 g, 3 mmol, Intermediate L) and 2,5-dibromopyridine (771.7 mg, 3.25 mmol) in DMA (20 mL) were added Pd₂(dba)₃ (248.6 mg, 0.27 mmol) and XantPhos (157.1 mg, 0.27 mmol) and Cs₂CO₃ (88.4 mg, 0.27 mmol) in turns at rt under air atmosphere. The resulting mixture was then stirred for 16 h at 120° C. under argon atmosphere. On completion, the reaction mixture was cooled to rt and filtration. The filtrate was concentrated under vacuum. The residue was purified by reverse phase flash chromatography (Column: WelFlash™ C18-I, 20-40 um, 330 g; Eluent A: Water (plus 10 mmol/L FA); Eluent B: ACN; Gradient: 25%-55% B in 25 min; Flow rate: 80 mL/min; Detector: 220/254 nm; desired fractions were collected at 50% B) and concentrated under reduced pressure to afford the title compound (1 g, 70% yield) as a white solid. LC/MS (ESI, m/z): [(M+H)]⁺=524.0, 526.0.

### Example 1.224. Preparation of 1-[8-(piperazin-1-yl)isoquinolin-4-yl]-1,3-diazinane-2,4-dione (Intermediate HS)

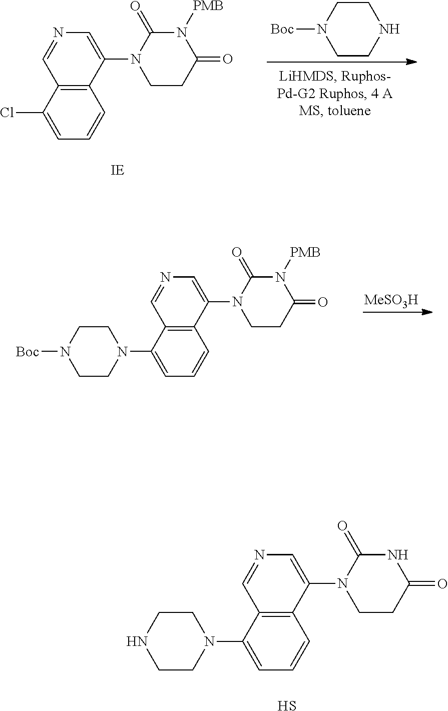

### Step 1—Tert-butyl 4-(4-{3-[(4-methoxyphenyl)methyl]-2,4-dioxo-1,3-diazinan-1-yl}isoquinolin-8-yl)piperazine-1-carboxylate

To a stirred solution of tert-butyl piperazine-1-carboxylate (1.04 g, 5.56 mmol) in toluene (20 mL) was added LiHMDS (1.69 g, 10.1 mmol) at 0° C. under nitrogen atmosphere. The resulting mixture was stirred for 1 h at 0° C. under nitrogen atmosphere. To the above mixture was added 1-(8-chloroisoquinolin-4-yl)-3-[(4-methoxyphenyl)methyl]-1,3-diazinane-2,4-dione (2 g, 5 mmol, Intermediate IE), RuPhos Palladacycle Gen. 3 (0.39 g, 0.51 mmol, CAS #1445085-77-7) at rt. The resulting mixture was stirred for additional 3 h at 80° C. On completion, the reaction mixture was concentrated under vacuum. The residue was purified by reverse phase flash chromatography (Column: WelFlash™ C18-I, 20-40 μm, 330 g; Eluent A: Water (plus 10 mmol/L NH4HCO3); Eluent B: ACN; Gradient: 30%-80% B in 30 min; Flow rate: 80 mL/min; Detector: 220/254 nm; desired fractions were collected at 75% B) and concentrated under reduced pressure to afford the title compound (2.2 g, 80% yield) as a light yellow solid. LC/MS (ESI, m/z): [(M+H)]⁺=546.2.

### Step 2—1-[8-(Piperazin-1-yl)isoquinolin-4-yl]-1,3-diazinane-2,4-dione

To a stirred solution of tert-butyl 4-(4-{3-[(4-methoxyphenyl)methyl]-2,4-dioxo-1,3-diazinan-1-yl}isoquinolin-8-yl)piperazine-1-carboxylate (2.2 g, 4.0 mmol) in CH₃SO₃H (20 mL) at rt under nitrogen atmosphere. The resulting mixture was stirred for 16 h at 80° C. under nitrogen atmosphere. On completion, the mixture was acidified neutralized to pH 7 with saturated NaHCO₃ (aq.). The residue was purified by reverse phase flash chromatography (Column: WelFlash™ C18-I, 20-40 μm, 330 g; Eluent A: Water (plus 10 mmol/L NH4HCO3); Eluent B: ACN; Gradient: 30%-95% B in 30 min; Flow rate: 80 mL/min; Detector: 220/254 nm; desired fractions were collected at 92% B) and concentrated under reduced pressure to afford the title compound (0.9 g, 69% yield) as a light yellow solid. LC/MS (ESI, m/z): [(M+H)]⁺=326.2.

### Example 1.225. Preparation of 1-(4-(1-(2,6-dioxopiperidin-3-yl)-3-methyl-2-oxo-2,3-dihydro-1H-benzo[d]imidazol-5-yl)phenyl)piperidine-4-carbaldehyde (Intermediate HT)

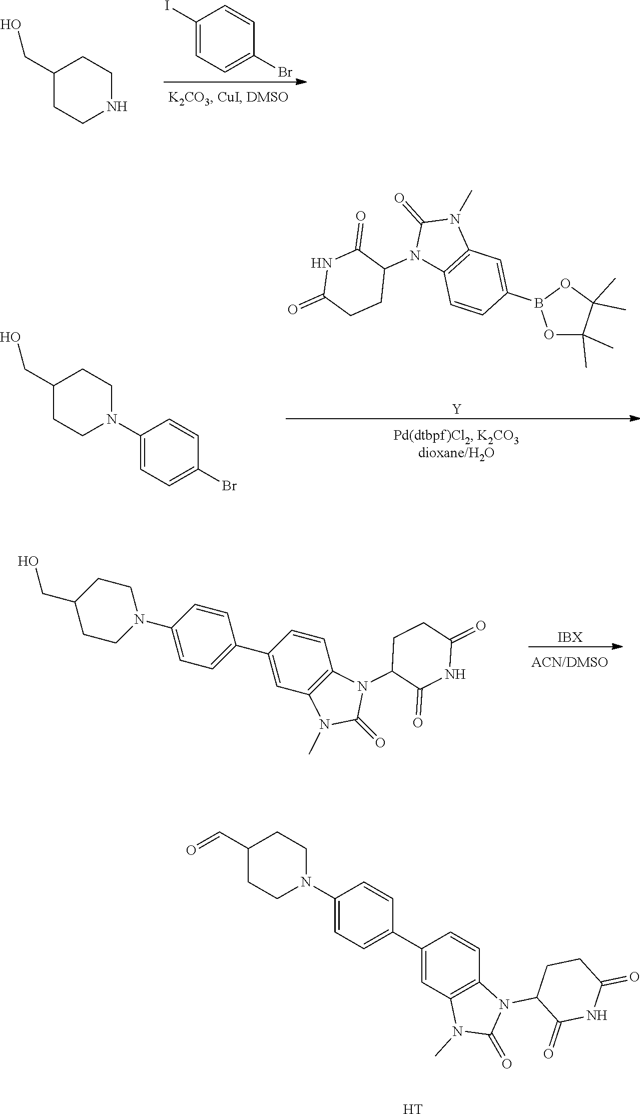

### Step 1—[1-(4-Bromophenyl)piperidin-4-yl]methanol

To a stirred solution of piperidin-4-ylmethanol (50 g, 434.118 mmol) and 4-bromoiodobenzene (147.38 g, 520.942 mmol) in DMSO (100 mL) were added K₂CO₃ (179.99 g, 1302.4 mmol) and CuI (8.27 g, 43.4 mmol) at rt under nitrogen atmosphere. The resulting mixture was stirred for 2 h at 90° C. under nitrogen atmosphere. On completion, the reaction mixture was cooled to rt and filtered, then the filter cake was washed with DCM (2×500 mL). The filtrate was concentrated under reduced pressure. The residue was triturated with water (500 mL) to afford the title compound (40 g, 34% yield) as a brown solid. LC/MS (ESI, m/z): [(M+H)]⁺=270.0, 272.0.

### Step 2—3-(5-{4-[4-(Hydroxymethyl)piperidin-1-yl]phenyl}-3-methyl-2-oxo-1,3-benzodiazol-1-yl)piperidine-2,6-dione

To a stirred solution of [1-(4-bromophenyl)piperidin-4-yl]methanol (5 g, 20 mmol) and 3-[3-methyl-2-oxo-5-(4,4,5,5-tetramethyl-1,3,2-dioxaborolan-2-yl)-1,3-benzodiazol-1-yl]piperidine-2,6-dione (7.13 g, 18.5 mmol, Intermediate Y) in dioxane (100 mL) and H₂O (20 mL) were added Pd(DtBPF)Cl2 (1.206 g, 1.85 mmol) and K₂CO₃ (5.115 g, 37.0 mmol) at rt under nitrogen atmosphere. The resulting mixture was stirred for 4 h at 80° C. under nitrogen atmosphere. On completion, the reaction mixture was cooled to rt and filtered, then the filter cake was washed with DCM (3×50 mL). The filtrate was adjusted pH to 4 with FA, and then concentrated under reduced pressure. The residue was purified by silica gel column chromatography, eluted with EA/DCM (1:1), to afford the title compound (1 g, 12% yield) as a brown oil. LC/MS (ESI, m/z): [(M+H)]⁺=449.2.

### Step 3—1-{4-[1-(2,6-dioxopiperidin-3-yl)-3-methyl-2-oxo-1,3-benzodiazol-5-yl]phenyl}piperidine-4-carbaldehyde

To a stirred solution of 3-(5-{4-[4-(hydroxymethyl)piperidin-1-yl]phenyl}-3-methyl-2-oxo-1,3-benzodiazol-1-yl)piperidine-2,6-dione (3 g, 7 mmol) in ACN (20 mL) and DMSO (30 mL) was added IBX (2.81 g, 10.0 mmol) in portions at rt under nitrogen atmosphere. The resulting mixture was stirred for 1 h at 80° C. under nitrogen atmosphere. On completion, the reaction mixture was cooled to and was acidified to pH 3 with FA. The resulting mixture was concentrated under vacuum. The residue was purified by silica gel column chromatography, eluted with CH₂Cl₂/MeOH (10:1), to afford (300 mg, 10% yield) as a brown oil. LC/MS (ESI, m/z): [(M+H)]⁺=447.2.

### Example 1.226. Preparation of N-[(1R,2S)-2-fluorocyclopropyl]-8-(methylamino)-6-{[3-(piperazin-1-yl)phenyl]amino}imidazo[1,2-b]pyridazine-3-carboxamide (Intermediate HU)

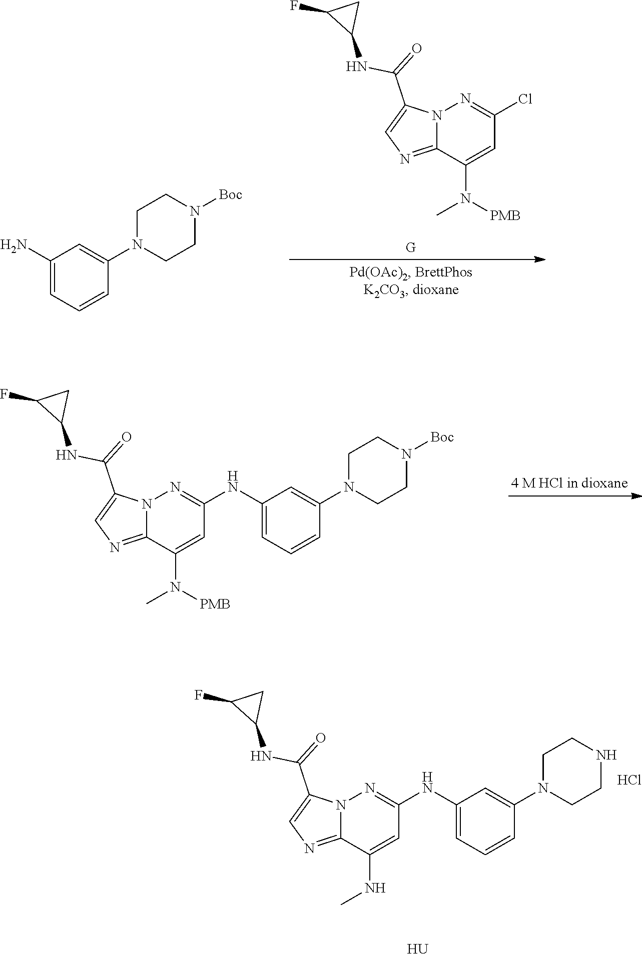

### Step 1—Tert-butyl 4-{3-[(3-{[(1R,2S)-2-fluorocyclopropyl]carbamoyl}-8-{[(4-methoxyphenyl)methyl](methyl)amino}imidazo[1,2-b]pyridazin-6-yl)amino]phenyl}piperazine-1-carboxylate

To a stirred mixture of tert-butyl 4-(3-aminophenyl)piperazine-1-carboxylate (3 g, 11 mmol, CAS #206879-72-3) and 6-chloro-N-[(1R,2S)-2-fluorocyclopropyl]-8-{[(4-methoxyphenyl)methyl](methyl)amino}imidazo[1,2-b]pyridazine-3-carboxamide (5.24 g, 13.0 mmol, Intermediate G) in dioxane (20.00 mL) were added Pd(OAc)₂ (0.24 g, 1.1 mmol) and BrettPhos (0.58 g, 1.1 mmol), K₂CO₃ (4.48 g, 32.5 mmol) at rt under nitrogen atmosphere. The resulting mixture was stirred for 16 h at 80° C. under nitrogen atmosphere. On completion, the reaction mixture was concentrated under vacuum. The residue was purified by reverse phase flash chromatography (Column: WelFlash™ C18-I, 20-40 ?m, 330 g; Eluent A: Water (plus 10 mmol/L NH₄HCO₃); Eluent B: ACN; Gradient: 25%-85% B in 30 min; Flow rate: 85 mL/min; Detector: 220/254 nm; desired fractions were collected at 80% B) and concentrated under reduced pressure to afford the title compound (5 g, 72% yield) as a light yellow solid. LC/MS (ESI, m/z): [(M+H)]⁺=645.2.

### Step 2—N-[(1R,2S)-2-fluorocyclopropyl]-8-(methylamino)-6-{[3-(piperazin-1-yl)phenyl]amino}imidazo[1,2-b]pyridazine-3-carboxamide Hydrochloride

To a stirred mixture of tert-butyl 4-{3-[(3-{[(1R,2S)-2-fluorocyclopropyl]carbamoyl}-8-{[(4-methoxyphenyl)methyl](methyl)amino}imidazo[1,2-b]pyridazin-6-yl)amino]phenyl}piperazine-1-carboxylate (2 g, 3 mmol) in 4 M HCl (gas) in 1,4-dioxane (20 mL) at rt under nitrogen atmosphere. The resulting mixture was stirred for 2 h at rt under nitrogen atmosphere. On completion, the reaction mixture was concentrated under vacuum to afford the title compound (2 g, 98% yield) as a light yellow solid. LC/MS (ESI, m/z): [(M+H)]⁺=425.2.

### Example 1.227. Preparation of 3-[4-(1,3-dioxolan-2-yl)piperidin-1-yl]aniline (Intermediate HV)

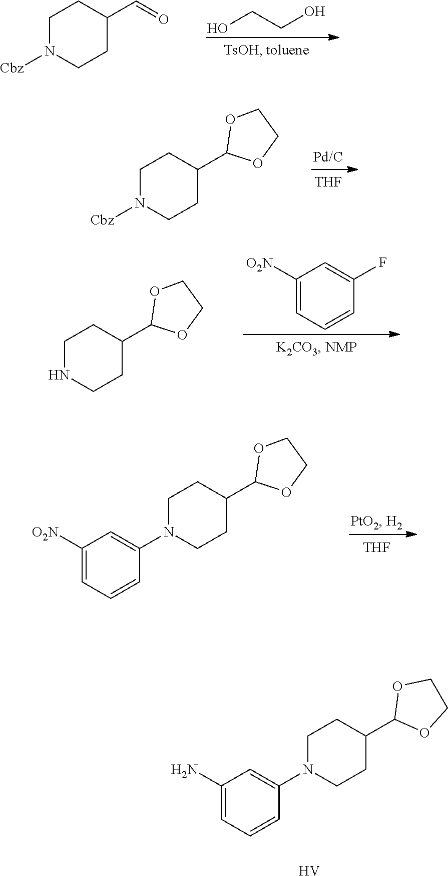

### Step 1—Benzyl 4-(1,3-dioxolan-2-yl)piperidine-1-carboxylate

To a stirred solution of benzyl 4-formylpiperidine-1-carboxylate (10 g, 40 mmol) in toluene (50 mL) and ethylene glycol (10 mL) was added TsOH (0.70 g, 4.0 mmol) at rt under nitrogen atmosphere. The resulting mixture was stirred for 2 h at 110° C. under nitrogen atmosphere. On completion, the reaction mixture was cooled to rt and concentrated under reduced pressure to afford the title compound (10 g, 85% yield) as a light brown oil. LC/MS (ESI, m/z): [(M+H)]⁺=292.2.

### Step 2—4-(1,3-Dioxolan-2-yl)piperidine

To a solution of benzyl 4-(1,3-dioxolan-2-yl)piperidine-1-carboxylate (10 g, 34 mmol) in THF (100 mL) was added Pd/C (0.37 g, 3.4 mmol) under nitrogen atmosphere. The reaction system was degassed under vacuum and purged with H₂ several times, then was hydrogenated under H₂ balloon (˜1 atm) at 25° C. for 16 h. After completion of the reaction, Pd/C was filtered off through celite. The filter cake was washed with THF (3×100 mL). The corresponding filtrate was concentrated under reduced pressure to provide the title compound (6 g, 95% yield) as a light brown oil. LC/MS (ESI, m/z): [(M+H)]⁺=158.1.

### Step 3—4-(1,3-Dioxolan-2-yl)-1-(3-nitrophenyl)piperidine

To a stirred solution of 3-fluoronitrobenzene (17.95 g, 127.2 mmol) and 4-(1,3-dioxolan-2-yl)piperidine (10 g, 60 mmol) in NMP (60 mL) was added K₂CO₃ (35.16 g, 254.4 mmol) at rt under nitrogen atmosphere. The resulting mixture was stirred for 16 h at 120° C. under nitrogen atmosphere. On completion, the reaction mixture was cooled to rt and concentrated under reduced pressure. The residue was purified by silica gel column chromatography, eluted with PE/EA (5:1), to afford the title compound (2 g, 11% yield) as a reddish brown semi-solid. LC/MS (ESI, m/z): [(M+H)]⁺=279.2.

### Step 4—3-[4-(1,3-Dioxolan-2-yl)piperidin-1-yl]aniline

To a solution of 4-(1,3-dioxolan-2-yl)-1-(3-nitrophenyl)piperidine (2 g, 7 mmol) in THF (20 mL) was added PtO₂ (0.16 g, 0.72 mmol) under nitrogen atmosphere. The reaction system was degassed under vacuum and purged with H₂ several times, then was hydrogenated under H₂ balloon (˜1 atm) at 25° C. for 2 h. After completion of the reaction, Pd/C was filtered off through celite. The filter cake was washed with MeOH (3×20 mL). The corresponding filtrate was concentrated under reduced pressure to provide the title compound (1.6 g, 90% yield) as a light brown solid. LC/MS (ESI, m/z): [(M+H)]⁺=249.2.

### Example 1.228. Preparation of N-[(1R,2S)-2-fluorocyclopropyl]-6-{[3-(4-formylpiperidin-1-yl)phenyl]amino}-8-(methylamino)imidazo[1,2-b]pyridazine-3-carboxamide (Intermediate HW)

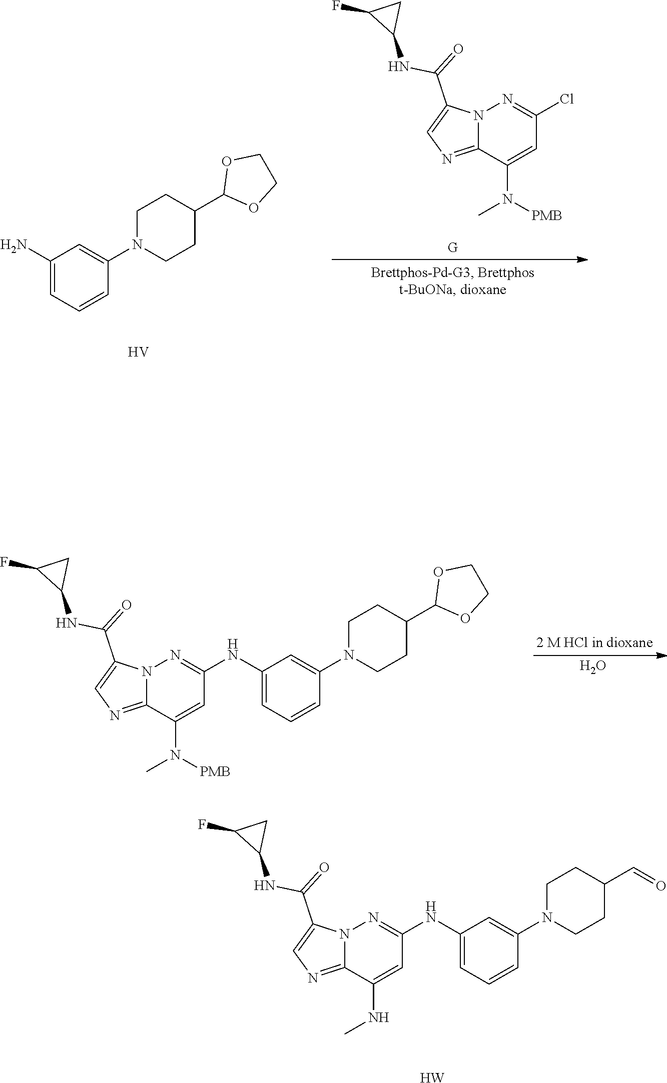

### Step 1—6-({3-[4-(1,3-Dioxolan-2-yl)piperidin-1-yl]phenyl}amino)-N-[(1R,2S)-2-fluorocyclopropyl]-8-{[(4-methoxyphenyl)methyl](methyl)amino}imidazo[1,2-b]pyridazine-3-carboxamide

To a stirred solution of 3-[4-(1,3-dioxolan-2-yl)piperidin-1-yl]aniline (1.6 g, 6.4 mmol, Intermediate HV) and 6-chloro-N-[(1R,2S)-2-fluorocyclopropyl]-8-{[(4-methoxyphenyl)methyl](methyl)amino}imidazo[1,2-b]pyridazine-3-carboxamide (3.12 g, 7.7 mmol, Intermediate G) in dioxane (20 mL) were added BrettPhos (0.35 g, 0.64 mmol) and K₂CO₃ (1.78 g, 12.9 mmol) at 25° C. under nitrogen atmosphere. To the above mixture was added Pd(AcO)₂ (0.14 g, 0.64 mmol) at rt under nitrogen atmosphere. The resulting mixture was stirred for additional 5 min at rt. The resulting mixture was stirred for 16 h at 80° C. under nitrogen atmosphere. On completion, the reaction mixture was cooled to rt and concentrated under reduced pressure. The residue was purified by silica gel column chromatography, eluted with PE/EA (1:1), to afford the title compound (1.9 g, 48% yield) as an off-white solid. LC/MS (ESI, m/z): [(M+H)]⁺=616.4.

### Step 2—N-[(1R,2S)-2-fluorocyclopropyl]-6-{[3-(4-formylpiperidin-1-yl)phenyl]amino}-8-(methylamino)imidazo[1,2-b]pyridazine-3-carboxamide

To a stirred solution of 6-({3-[4-(1,3-dioxolan-2-yl)piperidin-1-yl]phenyl}amino)-N-[(1R,2S)-2-fluorocyclopropyl]-8-{[(4-methoxyphenyl)methyl](methyl)amino}imidazo[1,2-b]pyridazine-3-carboxamide (500 mg, 0.8 mmol) in dioxane (4 mL) was added 2 M HCl (aq.). (4 mL) dropwise at rt under nitrogen atmosphere. The resulting mixture was stirred for 1 h at 50° C. under nitrogen atmosphere. On completion, the reaction mixture was cooled to rt and concentrated under reduced pressure. The residue was purified by reverse phase flash chromatography (Column: WelFlash™ C18-I, 20-40 μm, 120 g; Eluent A: Water (plus 5 mmol/L NH₄HCO₃); Eluent B: ACN; Gradient: 20%-50% B in 35 min; Flow rate: 60 mL/min; Detector: 220/254 nm; desired fractions were collected at 45% B) and concentrated under reduced pressure to afford the title compound (200 mg, 55% yield) as an off-white solid. LC/MS (ESI, m/z): [(M+H)]⁺=452.2.

### Example 1.229. Preparation of tert-butyl 7-(4-(4,4,5,5-tetramethyl-1,3,2-dioxaborolan-2-yl)phenyl)-2,7-diazaspiro[4.4]nonane-2-carboxylate (Intermediate HX)

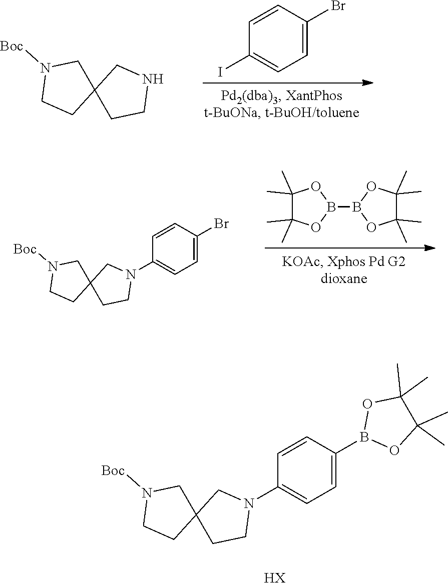

### Step 1—Tert-butyl 7-(4-bromophenyl)-2,7-diazaspiro[4.4]nonane-2-carboxylate

To a solution of tert-butyl 2,7-diazaspiro[4.4]nonane-2-carboxylate (4.00 g, 17.7 mmol, CAS #236406-49-8) and 1-bromo-4-iodobenzene (5 g, 17.7 mmol) in t-BuOH (100 mL) and Tol. (20 mL) was added t-BuONa (1.87 g, 19.4 mmol) and Pd₂(dba)₃ (1.62 g, 1.77 mmol), and Xantphos (2.05 g, 3.53 mmol). The mixture was stirred at 100° C. for 10 h. On completion, the reaction mixture was filtered and the filtrate was concentrated under reduced pressure to give a residue. The residue was purified by reversed-phase HPLC (column: Phenomenex luna C18 250*50 mm*15 um; mobile phase: [water (NH4HCO3)-ACN]; B %: 55%-85%, 20 min) to give the title compound (2.9 g, 42% yield) as yellow solid. LC-MS (ESI+) m/z 381.0 (M+H)⁺.

### Step 2—Tert-butyl 7-(4-(4,4,5,5-tetramethyl-1,3,2-dioxaborolan-2-yl)phenyl)-2,7-diazaspiro[4.4]nonane-2-carboxylate

To a solution of tert-butyl 7-(4-bromophenyl)-2,7-diazaspiro[4.4]nonane-2-carboxylate (2.4 g, 6.3 mmol) and 4,4,4′,4′,5,5,5′-heptamethyl-2,2′-bi(1,3,2-dioxaborolane) (3.20 g, 12.6 mmol) in 1,4-dioxane (25 mL) was added KOAc (1.24 g, 12.6 mmol) and XPHOS-PD-G2 (495 mg, 629 umol). The mixture was stirred at 90° C. for 7 hr. On completion, the reaction mixture was quenched with NH₄Cl (60 mL), and extracted with EtOAc (30 mL×3). The combined organic layer was washed with brine (30 mL), dried over Na₂SO₄, filtered and concentrated under reduced pressure to give a residue. The crude product was triturated with EtOAc 25° C. for 2 h then filtered. The filter cake was dried in vacuo to give the title compound (2.5 g, 74% yield) as gray solid. LC-MS (ESI+) m/z 429.0 (M+H)⁺.

### Example 1.230. Preparation of 3-(4-{2,7-Diazaspiro[3.5]nonan-7-yl}-3-methyl-2-oxo-1,3-benzodiazol-1-yl)piperidine-2,6-dione (Intermediate HY)

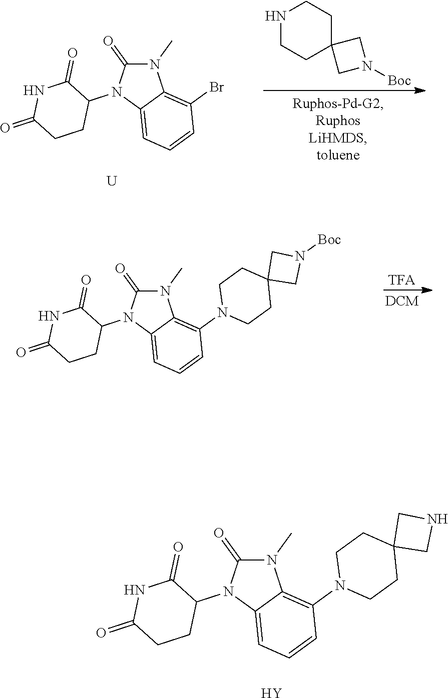

### Step 1—Tert-butyl 7-(1-(2,6-dioxopiperidin-3-yl)-3-methyl-2-oxo-2,3-dihydro-1H-benzo[d]imidazol-5-yl)-2,7-diazaspiro[3.5]nonane-2-carboxylate

To a solution of 3-(4-bromo-3-methyl-2-oxo-benzimidazol-1-yl)piperidine-2,6-dione (6 g, 17.74 mmol, Intermediate U), tert-butyl 2,7-diazaspiro[3.5]nonane-2-carboxylate (4.82 g, 21.3 mmol, CAS #236406-55-6), [2-(2-aminophenyl)phenyl]-chloro-palladium; dicyclohexyl-[2-(2, 6-diisopropoxyphenyl)phenyl]phosphane (2.76 g, 3.55 mmol), LiHMDS (1 M, 88.72 mL) and 4 Å molecular sieves (1.2 g) in toluene (120 mL) was added RuPhos (1.66 g, 3.55 mmol). The mixture was then stirred at 80° C. under N₂ for 2 hr. On completion, the mixture was adjusted to pH=5 with FA at 0° C., then filtered. The filtrate was concentrated under reduced pressure to give a residue. The residue was purified by column chromatography (SiO₂, PE:EA=2:1 to 0:1, P: Rf=0.4 (PE:EA=0:1)) to give the title compound (4.6 g, 50% yield) as off-white solid. ¹H NMR (400 MHz, CDCl₃-d) δ=8.11 (s, 1H), 7.02-6.98 (m, 1H), 6.90-6.88 (d, J=8.0 Hz, 1H), 6.59-6.57 (m, 1H), 5.23-5.19 (dd, J=5.2, 12.8 Hz, 1H), 3.76 (s, 5H), 3.71-3.64 (m, 2H), 3.11-3.08 (m, 2H), 2.93-2.92 (m, 1H), 2.82-2.69 (m, 4H), 2.24-2.22 (m, 1H), 1.95-1.92 (m, 4H), 1.47 (s, 9H).

### Step 2—3-(4-{2,7-Diazaspiro[3.5]nonan-7-yl}-3-methyl-2-oxo-1,3-benzodiazol-1-yl)piperidine-2,6-dione Trifluoroacetate

To a solution of tert-butyl 7-[1-(2,6-dioxopiperidin-3-yl)-3-methyl-2-oxo-1,3-benzodiazol-4-yl]-2,7-diazaspiro[3.5]nonane-2-carboxylate (70 mg, 0.2 mmol) (PF-0034-Boc) in DCM (5 mL) was added TFA (1 mL) dropwise at rt. The reaction mixture was stirred for 2 h at rt under nitrogen atmosphere. On completion, the reaction mixture was concentrated under reduced pressure. The residue was triturated with Et₂O to afford the title compound (50 mg) as a white solid. LC/MS (ESI, m/z): [(M+H)]⁺=384.2.

### Example 1.231. Preparation of 3-{3-methyl-2-oxo-4-[1-(piperidin-4-yl)azetidin-3-yl]-1,3-benzodiazol-1-yl}piperidine-2,6-dione (Intermediate HZ)

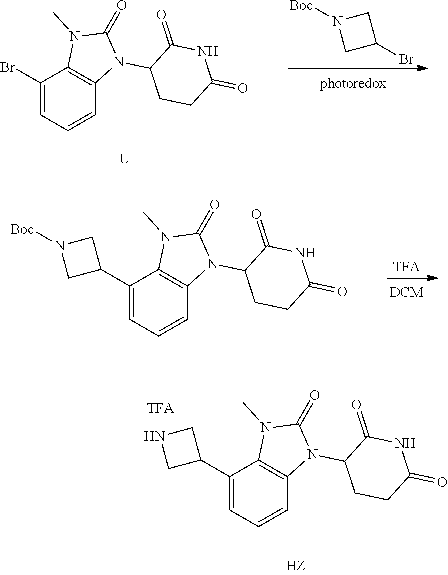

### Step 1—Tert-butyl 3-[1-(2,6-dioxopiperidin-3-yl)-3-methyl-2-oxo-1,3-benzodiazol-4-yl]azetidine-1-carboxylate

To an 80 ml vial equipped with a stir bar were added photocatalyst IR[DF(CF₃)PPY]₂(DTBPY)PF₆ (66.35 mg, 0.059 mmol), 3-(4-bromo-3-methyl-2-oxo-1,3-benzodiazol-1-yl)piperidine-2,6-dione (2 g, 6 mmol, Intermediate U), tert-butyl 3-bromoazetidine-1-carboxylate (1815.36 mg, 7.688 mmol), and tris(trimethylsilyl)silane (1470.68 mg, 5.914 mmol). The vial was sealed and placed under nitrogen before DME (20 mL) was added. To a separate vial was added NiCl₂ (3.83 mg, 0.030 mmol) and 2,6-lutidine (1267.51 mg, 11.828 mmol). The catalyst vial was sealed, purged with nitrogen then to it was added DME (20 mL). The precatalyst solution was sonicated and stirred for 5 min, after which was syringed into the reaction vessel. The solution was degassed by sparging with nitrogen while stirring for 10 minutes before sealing with Parafilm. The reaction was stirred and irradiated with a 34 W blue LED lamp for 16 h. On completion, the reaction mixture was concentrated under reduced pressure. The residue was dissolved in DCM (40 mL) and filtered. The filter cake was washed with DCM (3×40 mL). The filtrate was concentrated under reduced pressure. The residue was dissolved in water (40 mL). The resulting mixture was extracted with EtOAc (3×100 mL). The combined organic layers were washed with brine (1×100 mL), and dried over anhydrous Na₂SO₄. After filtration, the filtrate was concentrated under reduced pressure. The residue was purified by reverse phase flash chromatography (Column: WelFlash™ C18-I, 20-40 um, 330 g; Eluent A: Water (plus 10 mmol/L NH₄HCO₃); Eluent B: ACN; Gradient: 25%-55% B in 25 min; Flow rate: 80 mL/min; Detector: 220/254 nm; desired fractions were collected at 55% B) and concentrated under reduced pressure to afford the title compound (1 g, 41% yield) as a yellow solid. LC/MS (ESI, m/z): [(M+H)]⁺=415.2.

### Step 2—3-[4-(Azetidin-3-yl)-3-methyl-2-oxo-1,3-benzodiazol-1-yl]piperidine-2,6-dione Trifluoroacetate

To a stirred mixture of tert-butyl 3-[1-(2,6-dioxopiperidin-3-yl)-3-methyl-2-oxo-1,3-benzodiazol-4-yl]azetidine-1-carboxylate (1.5 g, 3.6 mmol) in DCM (16 mL) was added TFA (4 mL) dropwise at rt. The resulting mixture was stirred for 1 h at rt under air atmosphere. On completion, the reaction mixture was concentrated in vacuo. The residue was purified by trituration with Et2O (20 mL) to afford the title compound (600 mg, 40% yield) as a white solid. LC/MS (ESI, m/z): [(M+H)]⁺=315.2.

### Example 1.232. Preparation of tert-butyl N-(non-8-yn-1-yl)carbamate (Intermediate IA)

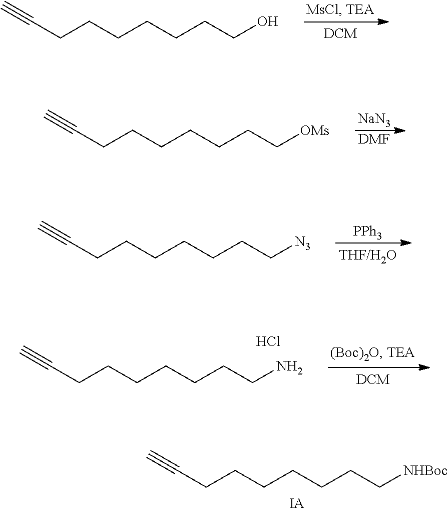

### Step 1—Non-8-yn-1-yl methanesulfonate

To a stirred solution of non-8-yn-1-ol (20.00 g, 142.6 mmol) and TEA (39.54 mL, 285.3 mmol) in DCM (200.00 mL) was added MsCl (24.51 g, 213.9 mmol) at 0° C. under nitrogen atmosphere. The mixture was stirred for 1 h at rt. On completion, the reaction was quenched with water (150 mL) at rt. The resulting mixture was extracted with CH₂Cl₂ (2×50 mL). The combined organic layers were washed with NaCl aq (1×100 mL), and dried over anhydrous Na₂SO₄. After filtration, the filtrate was concentrated under reduced pressure to give the title compound (30 g, 96% yield) as a light yellow oil, ¹H NMR (400 MHz, DMSO-d₆) δ 4.19 (t, J=6.5 Hz, 2H), 3.16 (s, 3H), 2.73 (t, J=2.7 Hz, 1H), 2.16 (td, J=6.9, 2.7 Hz, 2H), 1.70-1.62 (m, 2H), 1.49-1.42 (m, 2H), 1.40-1.26 (m, 6H).

### Step 2—9-Azidonon-1-yne

To a stirred solution of non-8-yn-1-yl methanesulfonate (30.00 g, 137.4 mmol) in DMF (100.00 mL) was added NaN₃ (17.87 g, 274.8 mmol) at rt under nitrogen atmosphere. The mixture was stirred for 16 h at 55° C. On completion, the reaction was quenched with water at rt. The resulting mixture was extracted with EtOAc (3×200 mL). The combined organic layers were washed with brine (1×100 mL), and dried over anhydrous Na₂SO₄. After filtration, the filtrate was concentrated under reduced pressure. The residue was purified by silica gel column chromatography, eluted with PE/EtOAc (30:1) to afford the title compound (21 g, 93% yield) as a colorless oil. ¹H NMR (400 MHz, DMSO-d₆) δ 3.37-3.27 (m, 2H), 2.73 (t, J=2.8 Hz, 1H), 2.14 (td, J=6.9, 2.6 Hz, 2H), 1.56-1.49 (m, 2H), 1.47-1.40 (m, 2H), 1.39-1.24 (m, 6H).

### Step 3—Non-8-yn-1-amine Hydrochloride

To a stirred solution of 9-azidonon-1-yne (32.00 g, 193.7 mmol) in THF (300.00 mL)/H₂O (30.00 mL) was added PPh₃ (76.19 g, 290.5 mmol) in portions at 0° C. under nitrogen atmosphere. The solution was stirred for 4 h at 55° C. On completion of the reaction, the solution was concentrated under reduced pressure and diluted with DCM (300 mL). The mixture was acidified to pH 1 with HCl (2 M aq.). The aqueous layer was extracted with DCM (3×100 mL). The aqueous layer was concentrated under reduced pressure to give the title compound (27 g, 79% yield) as a white solid. ¹H NMR (400 MHz, DMSO-d₆) δ 8.04 (s, 3H), 2.81-2.68 (m, 3H), 2.15 (td, J=6.9, 2.7 Hz, 2H), 1.59-1.51 (m, 2H), 1.50-1.39 (m, 2H), 1.39-1.23 (m, 6H). LC/MS (ESI, m/z): [(M+1)]⁺=140.2.

### Step 4—Tert-butyl N-(non-8-yn-1-yl)carbamate

To a stirred mixture of non-8-yn-1-amine hydrochloride (27.00 g, 153.7 mmol) in DCM (300.00 mL) was added TEA (106.5 mL, 768.4 mmol) dropwise at 0° C. under nitrogen atmosphere. Then Boc₂O (50.31 g, 230.5 mmol) was added dropwise at 0° C. under nitrogen atmosphere. The mixture was stirred for 16 h at rt. On completion, the reaction was quenched with water (200 mL) at rt. The resulting mixture was extracted with CH₂Cl₂ (2×100 mL). The combined organic layers were dried over anhydrous Na₂SO₄. After filtration, the filtrate was concentrated under reduced pressure. The residue was purified by silica gel column chromatography, eluted with PE/EtOAc (3:1) to afford the title compound (20 g, 54% yield) as a colorless oil. ¹H NMR (400 MHz, DMSO-d₆) δ 6.76 (t, J=5.7 Hz, 1H), 2.90 (q, J=6.6 Hz, 2H), 2.73 (t, J=2.6 Hz, 1H), 2.15 (td, J=7.0, 2.7 Hz, 2H), 1.48-1.41 (m, 2H), 1.41-1.32 (m, 13H), 1.30-1.17 (m, 4H). LC/MS (ESI, m/z): [(M+1-56)]⁺=184.2.

### Example 1.233. Preparation of Tert-butyl methyl(3-(piperidin-4-yloxy)propyl)carbamate (Intermediate IB)

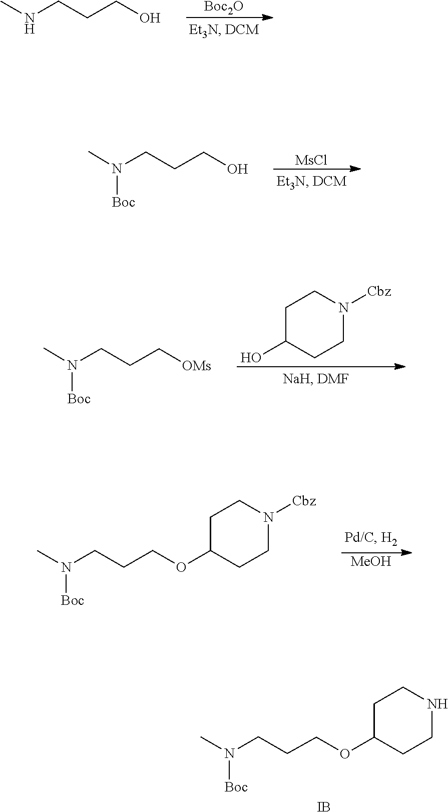

### Step 1—Tert-butyl (3-hydroxypropyl)(methyl)carbamate

To a solution of 3-(methylamino) propan-1-ol (15 g, 170 mmol) and Et₃N (20.43 g, 201.9 mmol, 28.11 mL) in DCM (150 mL) was added Boc₂O (42.00 g, 192.4 mmol, 44.21 mL) at 0° C. slowly. Then the mixture was stirred at 20° C. for 15 hr. On completion, the reaction mixture was added to ice-water (300 mL), and extracted with DCM (300 mL×3). The combined organic phase was washed with brine (250 mL×3), dried with anhydrous Na2SO4, filtered and concentrated in vacuo. The residue was purified by column chromatography (SiO₂, Petroleum ether/Ethyl acetate=10/1 to 0/1) to afford the title compound (28.2 g, 89% yield) as yellow oil. ¹H NMR (400 MHz, CHLOROFORM-d) δ=3.49 (m, 2H), 3.33-3.30 (t, J=6.0 Hz, 2H), 2.77 (s, 3H), 2.59-2.41 (br, 1H), 1.64-1.56 (m, 2H), 1.40-1.38 (s, 9H).

### Step 2—3-((Tert-butoxycarbonyl)(methyl)amino)propyl methanesulfonate

To a solution of tert-butyl N-(3-hydroxypropyl)-N-methyl-carbamate (28.2 g, 149 mmol) and Et₃N (19.60 g, 193.7 mmol, 26.96 mL) in DCM (250 mL) was added MsCl (19.64 g, 171.5 mmol, 13.27 mL) at 0° C. The mixture was stirred at 20° C. for 2 h. On completion, the reaction mixture was added to ice-water (200 mL), and it was extracted with DCM (150 mL×3). The combined organic phase was washed with brine (125 mL×3), dried with anhydrous Na₂SO₄, filtered and concentrated in vacuo to afford the title compound (39.8 g, quant. yield as yellow oil. ¹H NMR (400 MHz, CHLOROFORM-d) δ=4.20-4.17 (t, J=6.0 Hz, 2H), 3.30-3.26 (t, J=6.8 Hz, 2H), 2.96 (s, 3H), 2.80 (s, 3H), 1.94-1.88 (m, 2H), 1.39 (s, 9H).

### Step 3—Benzyl 4-(3-((tert-butoxycarbonyl)(methyl)amino)propoxy)piperidine-1-carboxylate

To a solution of benzyl 4-hydroxypiperidine-1-carboxylate (30.80 g, 130.9 mmol) in DMF (250 mL) was added NaH (7.00 g, 175 mmol, 60% dispersion in mineral oil) at 0° C. slowly. The mixture was stirred at 20° C. for 0.5 h, and a solution of 3-[tertbutoxycarbonyl(methyl)amino]propyl methanesulfonate (28 g, 100 mmol) in DMF (20 mL) was added to above mixture at 20° C. The mixture was then stirred at 20° C. for 2 h. On completion, the reaction mixture was added to ice-water (1 L), and it was extracted with ethyl acetate (1 L×3). The combined organic phase was washed with brine (800 mL×4), dried with anhydrous Na₂SO₄, filtered and concentrated in vacuo. The residue was purified by column chromatography (SiO₂, Petroleum ether/Ethyl acetate=100/1 to 1/1) to give the title compound (13.0 g, 31% yield) as yellow oil. ¹H NMR (400 MHz, CHLOROFORM-d) δ=7.35 (m, 5H), 5.13 (s, 2H), 3.87-3.71 (m, 2H), 3.47-3.44 (m, 3H), 3.31-3.16 (m, 4H), 2.86 (s, 3H), 1.82-1.77 (m, 4H), 1.54-1.53 (m, 2H), 1.46 (s, 9H), 1.28-1.24 (m, 1H).

### Step 4—Tert-butyl methyl(3-(piperidin-4-yloxy)propyl)carbamate

To a solution of benzyl 4-[3-[tert-butoxycarbonyl(methyl)amino]propoxy]piperidine-1-carboxylate (9 g, 20 mmol) in MeOH (108 mL) was added Pd/C (3.0 g, 10 wt %) in one portion at 25° C. The resulting mixture was degassed with H₂ three times, then stirred at 25° C. for 24 hrs under H₂ (50 psi). On completion, the residue was filtered, and the filtrate was concentrated in vacuo to give the title compound (5.7 g, 95% yield) as a yellow oil. ¹H NMR (400 MHz, CHLOROFORM-d) δ=3.47-3.43 (m, 2H), 3.41-3.33 (m, 1H), 3.28 (m, 2H), 3.12-3.09 (m, 2H), 2.85 (s, 3H), 2.79 (m, 1H), 2.71-2.69 (m, 1H), 1.79-1.76 (m, 4H), 1.53-1.49 (m, 2H), 1.46 (s, 9H).

### Example 1.234. Preparation of (Intermediate IC)

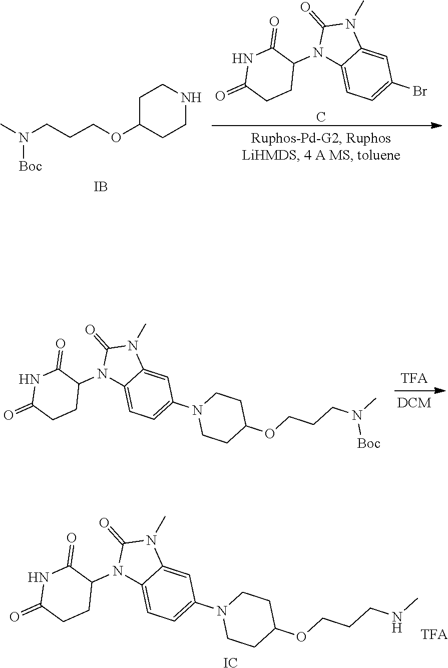

### Step 1—Tert-butyl (3-((1-(1-(2,6-dioxopiperidin-3-yl)-3-methyl-2-oxo-2,3-dihydro-1H-benzo[d]imidazol-5-yl)piperidin-4-yl)oxy)propyl)(methyl)carbamate

To a solution of tert-butyl N-methyl-N-[3-(4-piperidyloxy)propyl]carbamate (5.76 g, 21.1 mmol, Intermediate IB), 3-(5-bromo-3-methyl-2-oxo-benzimidazol-1-yl)piperidine-2,6-dione (5.5 g, 16 mmol, Intermediate C), 4 Å molecular sieves (1.2 g), RuPhos (1.52 g, 3.25 mmol) and LiHMDS (1.0 M, 97.59 mL) in toluene (110 mL) was added [2-(2-aminophenyl)phenyl]-chloro-palladium; dicyclohexyl-[2-(2,6-diiso propoxyphenyl) phenyl]phosphane (2.53 g, 3.25 mmol) in one portion at 25° C., then the resulting mixture was stirred at 80° C. for 2 hrs under N₂. On completion, the reaction mixture was adjusted to pH=5 with FA at 0° C., then filtered, and the filtrate was concentrated under reduced pressure. The residue was triturated with MTBE (30 mL), then filtered, and the filtered cake was purified by flash silica gel chromatography (Biotage®; 45 g SepaFlash® Silica Flash Column, Eluent of 20˜100% Ethyl acetate/Petroleum ether gradient @ 60 mL/min). The filtrate was repurified by flash silica gel chromatography (Biotage®; 45 g SepaFlash® Silica Flash Column, Eluent of 20˜100% Ethyl acetate/Petroleum ether gradient @ 60 mL/min) to give the title compound (2.70 g, 31% yield) as a yellow solid. ¹H NMR (400 MHz, CHLOROFORM-d) δ=8.29 (br s, 1H), 6.73-6.63 (m, 3H), 5.21-5.17 (dd, J=5.2, 12.8 Hz, 1H), 3.51-3.48 (t, J=6.4 Hz, 2H), 3.44-3.42 (m, 3H), 3.41 (s, 3H), 3.37-3.25 (m, 2H), 2.91-2.87 (m, 6H), 2.84-2.76 (m, 1H), 2.69 (m, 1H), 2.27-2.15 (m, 1H), 2.07-1.97 (m, 2H), 1.83-1.69 (m, 5H), 1.46 (s, 9H). LC-MS (ESI⁺) m/z 530.3 (M+1)⁺.

### Step 2—3-(3-Methyl-5-{4-[3-(methylamino)propoxy]piperidin-1-yl}-2-oxo-1,3-benzodiazol-1-yl) piperidine-2,6-dione Trifluoroacetate

To a stirred solution of tert-butyl N-[3-({1-[1-(2,6-dioxopiperidin-3-yl)-3-methyl-2-oxo-1,3-benzodiazol-5-yl]piperidin-4-yl}oxy)propyl]-N-methylcarbamate (80.0 mg, 0.094 mmol) in DCM (5 mL) was added TFA (1 mL) at rt. The resulting mixture was stirred for 1 h at rt. On completion, the resulting mixture was concentrated under reduced pressure. The residue was triturated with Et₂O to afford the title compound (85.2 mg) as brown semi-solid. LC/MS (ESI, m/z): [(M+H)]⁺=430.2.

### Example 1.235. Preparation of 3-(4-Methoxybenzyl)dihydropyrimidine-2,4(1H,3H)-dione (Intermediate ID)

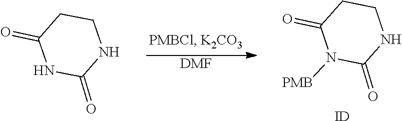

To a mixture of dihydropyrimidine-2,4(1H,3H)-dione (10.0 g, 87.6 mmol, CAS #504-07-4) in DMF (100 mL) was added PMB-Cl (13.7 g, 87.6 mmol, 11.9 mL), Cs₂CO₃ (28.5 g, 87.6 mmol) at 25° C. Then the mixture was stirred at 50° C. for 3 hours. On completion, the reaction mixture was quenched with of water (100 mL), and extracted with EtOAc (3×50 mL). The organic layer was dried over anhydrous Na₂SO₄, filtered and concentrated in vacuo. The crude product was purified by re-crystallization from EA/PE (20 mL, v/v=1/1) at 25° C. to give the title compound (9.40 g, 45% yield) as a white solid. ¹H NMR (400 MHz, CDCl₃) δ 7.81 (s, 1H), 7.18 (d, J=8.4 Hz, 2H), 6.83 (d, J=8.4 Hz, 2H), 4.72 (s, 2H), 3.72 (s, 3H), 3.23-3.20 (m, 2H), 2.63 (t, J=6.8 Hz, 2H).

### Example 1.236. Preparation of 1-(8-Chloro-4-isoquinolyl)hexahydropyrimidine-2,4-dione (Intermediate IE)

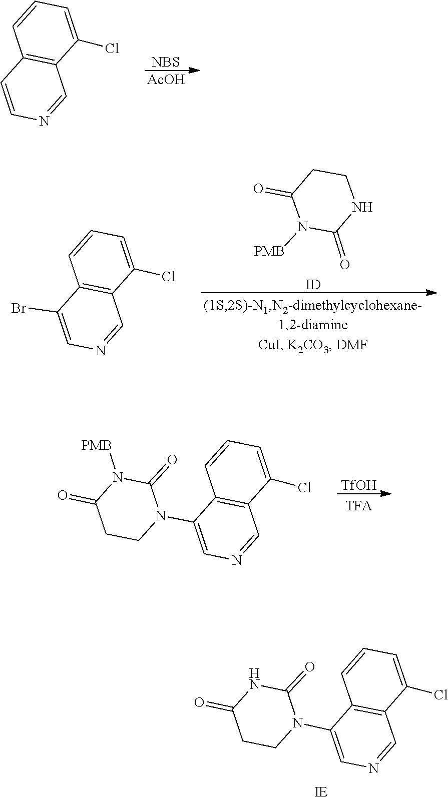

### Step 1—4-Bromo-8-chloro-isoquinoline

To a solution of 8-chloroisoquinoline (5.00 g, 30.5 mmol, CAS #34784-07-1) in AcOH (50 mL) was added NBS (7.07 g, 39.7 mmol), then the reaction mixture was stirred at 50° C. for 40 min. On completion, the reaction mixture was diluted with water (100 mL), then extracted with EA (3×80 mL). The combined organic layer was basified with NaHCO₃ until the pH=6-7, then the mixture was extracted with EA (2×60 mL). The combined organic layer was dried over Na₂SO₄, filtered and concentrated in vacuo to give a residue. The residue was purified by column chromatography (SiO₂, PE:EA=100:1 to PE:EA=50:1, PE:EA=10:1, P1:Rf=0.74) to give the title compound (1.00 g, 37% yield) as yellow solid. ¹HNMR (400 MHz, CDCl₃) δ 9.56 (s, 1H), 8.78 (s, 1H), 8.10-8.03 (m, 1H), 7.73-7.64 (m, 2H). LC-MS (ESI⁺) m/z 241.9 (M+H)⁺.

### Step 2—1-(8-Chloro-4-isoquinolyl)-3-[(4-methoxyphenyl)methyl]hexahydropyrimidine-2,4-dione

To a solution of 4-bromo-8-chloro-isoquinoline (100 mg, 400 umol) and 3-[(4-methoxyphenyl)methyl]hexahydropyrimidine-2,4-dione (96.6 mg, 412 umol, Intermediate ID) in DMF (1 mL) was added CuI (7.85 mg, 41.2 umol), (1S,2S)—N₁,N₂-dimethylcyclohexane-1,2-diamine (5.87 mg, 41.2 umol) and K₃PO₄ (175 mg, 824 umol), then the mixture was stirred at 110° C. for 8 hr. On completion, the reaction mixture was filtered and concentrated in vacuo to give the residue. The residue was diluted with water (50 mL) and extracted with EA (5×30 mL). Then the combined organic layers was dried over anhydrous Na₂SO₄, filtered and concentrated in vacuo to give a residue. The residue was purified by reverse-phase (0.1% FA) to give the title compound (15 mg, 3% yield) as yellow solid. ¹H NMR (400 MHz, DMSO-d₆) δ 9.89-9.56 (br s, 1H), 8.59 (br s, 1H), 7.73-7.68 (m, 1H), 7.64 (t, J=8.0 Hz, 1H), 7.60-7.55 (m, 1H), 7.43 (d, J=8.4 Hz, 2H), 6.85 (d, J=8.4 Hz, 2H), 5.00 (s, 2H), 3.95-3.86 (m, 1H), 3.80 (s, 3H), 3.78-3.69 (m, 1H), 3.07-2.99 (m, 2H); LC-MS (ESI⁺) m/z 396.1 (M+H)⁺.

### Step 3—1-(8-Chloro-4-isoquinolyl)hexahydropyrimidine-2,4-dione

A solution of 1-(8-chloro-4-isoquinolyl)-3-[(4-methoxyphenyl)methyl]hexahydropyrimidine-2,4-dione (40.0 mg, 101 umol) in TFA (0.49 mL) and TfOH (0.01 mL) was stirred at 60° C. for 2 hours. On completion, the mixture was concentrated to give the residue and purified by prep-HPLC (0.1% FA) to give the title compound (3 mg, 11% yield) as yellow solid. ¹H NMR (400 MHz, DMSO-d₆) δ=10.59 (s, 1H), 9.56 (s, 1H), 8.71 (s, 1H), 8.03 (d, J=8.4 Hz, 1H), 7.92-7.87 (m, 1H), 7.85-7.78 (m, 1H), 4.00-3.93 (m, 1H), 3.75-3.69 (m 1H), 3.03-2.95 (m, 1H), 2.79-2.72 (m, 1H). LC-MS (ESI⁺) m/z 276.0 (M+H)⁺.

### Example 1.237. Preparation of 3-{3-Methyl-2-oxo-4-[4-(piperidin-4-yl)piperazin-1-yl]-1,3-benzodiazol-1-yl}piperidine-2,6-dione (Intermediate IF)

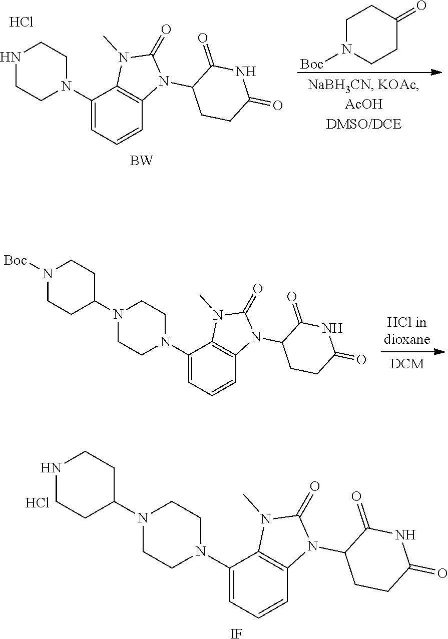

### Step 1—Tert-butyl 4-{4-[1-(2,6-dioxopiperidin-3-yl)-3-methyl-2-oxo-1,3-benzodiazol-4-yl]piperazin-1-yl}piperidine-1-carboxylate

To a stirred solution of 3-[3-methyl-2-oxo-4-(piperazin-1-yl)-1,3-benzodiazol-1-yl]piperidine-2,6-dione hydrochloride (450 mg, 1.2 mmol, Intermediate BW) and tert-butyl 4-oxopiperidine-1-carboxylate (283.26 mg, 1.422 mmol) in DMSO (4 mL) and DCE (4 mL) were added KOAc (348.80 mg, 3.555 mmol) and AcOH (0.35 mL, 6.108 mmol) in portions. The resulting mixture was stirred for 30 min at rt. Then to above mixture was added NaBH₃CN (372.23 mg, 5.925 mmol) in portions at 0° C. under nitrogen atmosphere. The resulting mixture was stirred for 2 h at rt under air atmosphere. On completion, the reaction mixture was concentrated under vacuum. The crude product was purified by reverse phase flash (Column: WelFlash™ C18-I, 20-40 um, 330 g; Eluent A: Water (plus 10 mmol/L NH₄HCO₃); Eluent B: ACN; Gradient: 25%-55% B in 35 min; Flow rate: 80 mL/min; Detector: 254 nm; desired fractions were collected at 32% B) and concentrated under reduced pressure to afford the title compound (80 mg, 13% yield) as a light brown solid. LC/MS (ESI, m/z): [(M+H)]⁺=527.3.

### Step 2—3-{3-Methyl-2-oxo-4-[4-(piperidin-4-yl)piperazin-1-yl]-1,3-benzodiazol-1-yl}piperidine-2,6-dione Trifluoroacetate

To a stirred solution of tert-butyl 4-{4-[1-(2,6-dioxopiperidin-3-yl)-3-methyl-2-oxo-1,3-benzodiazol-4-yl]piperazin-1-yl}piperidine-1-carboxylate (80 mg, 0.152 mmol) in DCM (6 mL) was added TFA (2 mL) dropwise at rt. The resulting mixture was stirred for 1 h at rt. On completion, the reaction mixture was concentrated under vacuum. The residue was triturated with Et₂O (3 mL) to afford the title compound (75 mg) as an off-white solid. LC/MS (ESI, m/z): [(M+H)]⁺=427.3.

### Example 1.238. Preparation of 3-[4-(4-{[1,4′-bipiperidin]-4-yl}piperazin-1-yl)-3-methyl-2-oxo-1,3-benzodiazol-1-yl]piperidine-2,6-dione (Intermediate IG)

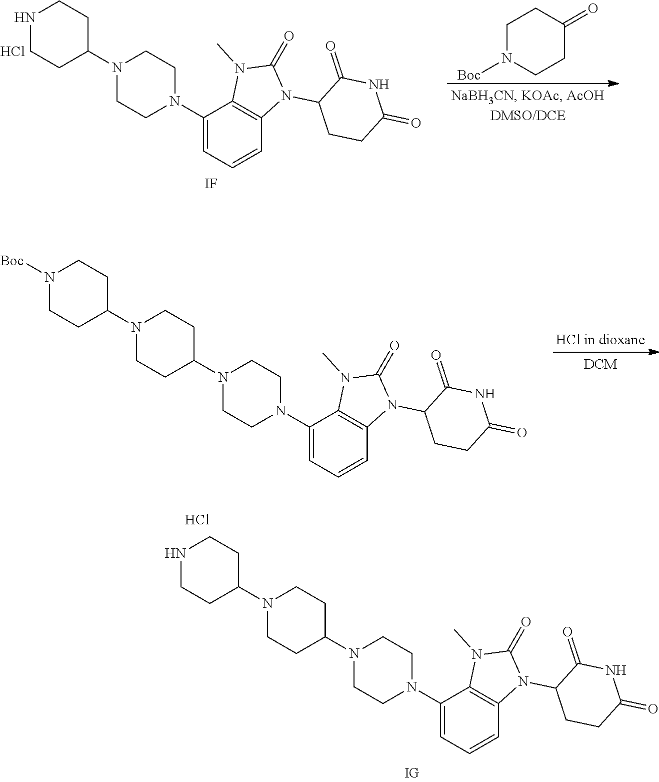

### Step 1—Tert-butyl 4-{4-[1-(2,6-dioxopiperidin-3-yl)-3-methyl-2-oxo-1,3-benzodiazol-4-yl]piperazin-1-yl}-[1,4′-bipiperidine]-1′-carboxylate

To a stirred solution of 3-{3-methyl-2-oxo-4-[4-(piperidin-4-yl)piperazin-1-yl]-1,3-benzodiazol-1-yl}piperidine-2,6-dione trifluoroacetate (75 mg, 0.14 mmol, Intermediate IF) and tert-butyl 4-oxopiperidine-1-carboxylate (42.04 mg, 0.211 mmol) in DMSO (2.5 mL) and DCE (2.5 mL) were added KOAc (51.77 mg, 0.528 mmol) and AcOH (0.06 mL, 1.047 mmol) in turns at rt under nitrogen atmosphere. The resulting mixture was stirred for 30 min at rt. To above mixture was added NaBH₃CN (55.25 mg, 0.880 mmol) in portions at 0° C. under nitrogen atmosphere. The resulting mixture was stirred for 2 h at rt under air atmosphere. On completion, the reaction mixture was concentrated under vacuum. The crude product was purified by reverse phase flash (Column: WelFlash™ C18-I, 20-40 um, 40 g; Eluent A: Water (10 mmol/L NH₄HCO₃); Eluent B: ACN; Gradient: 25%-60% B in 35 min; Flow rate: 40 mL/min; Detector: 254 nm; desired fractions were collected at 48% B) and concentrated under reduced pressure to afford the title compound (40 mg, 37% yield) as an off-white solid. LC/MS (ESI, m/z): [(M+H)]⁺=610.4.

### Step 2—3-[4-(4-{[1,4′-Bipiperidin]-4-yl}piperazin-1-yl)-3-methyl-2-oxo-1,3-benzodiazol-1-yl]piperidine-2,6-dione Hydrochloride

To a solution of tert-butyl 4-{4-[1-(2,6-dioxopiperidin-3-yl)-3-methyl-2-oxo-1,3-benzodiazol-4-yl]piperazin-1-yl}-[1,4′-bipiperidine]-1′-carboxylate (40 mg, 0.066 mmol) in DCM (1.5 mL) was added 4 M HCl (gas) in 1,4-dioxane (1 mL). The resulting mixture was stirred for 1 h at rt under nitrogen atmosphere. On completion, the reaction mixture was concentrated under vacuum. The residue was triturated with Et₂O to afford the title compound (35 mg, 98% yield) as an off-white solid. LC/MS (ESI, m/z): [(M+H)]⁺=510.4.

### Example 1.239. Preparation of Tert-butyl 4-[(3R)-pyrrolidin-3-yl]piperidine-1-carboxylate (Intermediate IH)

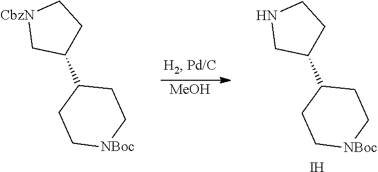

To a suspension of Pd/C (0.8 g, 3.86 mmol, 10 wt %) in methanol (12 mL) was added a solution of tert-butyl 4-[(3R)-1-benzyloxycarbonylpyrrolidin-3-yl]piperidine-1-carboxylate (1.5 g, 3.9 mmol, synthesized via Step 1 of Intermediate GS) in methanol (12 mL) under nitrogen. Then it was degassed and purged with hydrogen three times, then the mixture was stirred at 25° C. for 12 hours under hydrogen (15 psi). On completion, the reaction mixture was filtered and the filtrate was concentrated under reduced pressure to give the title compound (800 mg) as a white solid. LC-MS (ESI+) m/z 255.5 (M+H)⁺.

### Example 1.240. Preparation of tert-butyl 7-(2-chloropyrimidin-5-yl)-6-oxo-2,7-diazaspiro[4.4]nonane-2-carboxylate (Intermediate II)

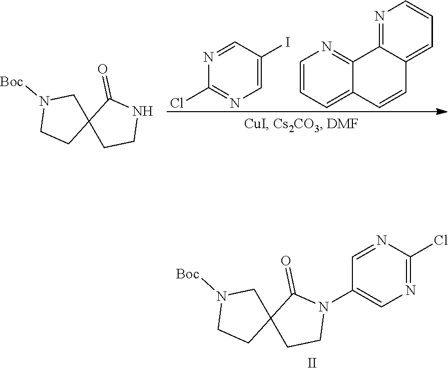

A solution of tert-butyl 6-oxo-2,7-diazaspiro[4.4]nonane-2-carboxylate (4.00 g, 16.7 mmol, CAS #1194376-44-7), 2-chloro-5-iodopyrimidine (4.40 g, 18.3 mmol, CAS #32779-38-7) and CuI (4.12 g, 21.6 mmol) in DMF (40 mL) was degassed and purged with N₂ gas. Then, Cs₂CO₃ (10.9 g, 33.3 mmol) and 1,10-phenanthroline (900 mg, 4.99 mmol) was added sequentially and the resulting mixture was stirred at 90° C. for 12 hrs. On completion, the reaction mixture was quenched with NH₄Cl (30 mL) and extracted with EtOAc (40 mL×2). The combined organic layers were washed with brine (40 mL×3), and dried over Na₂SO₄ and evaporated. The residue was prep-TLC (Petroleum ether/Ethyl acetate=1:1) to give the title compound (4.59 g, 78% yield) as a white solid. ¹H NMR (400 MHz, DMSO-d₆) δ 9.11 (s, 2H), 3.88 (t, J=6.8 Hz, 2H), 3.51-3.40 (m, 2H), 3.30 (br s, 2H), 2.89 (s, 1H), 2.73 (s, 1H), 2.22-2.13 (m, 2H), 2.11-2.04 (m, 1H), 1.95-1.87 (m, 1H), 1.38-1.37 (m, 1H), 1.40 (s, 8H).

### Example 1.241. Preparation of 3-(5-ethynyl-3-methyl-2-oxo-2,3-dihydro-1H-benzo[d]imidazol-1-yl)piperidine-2,6-dione (Intermediate IJ)

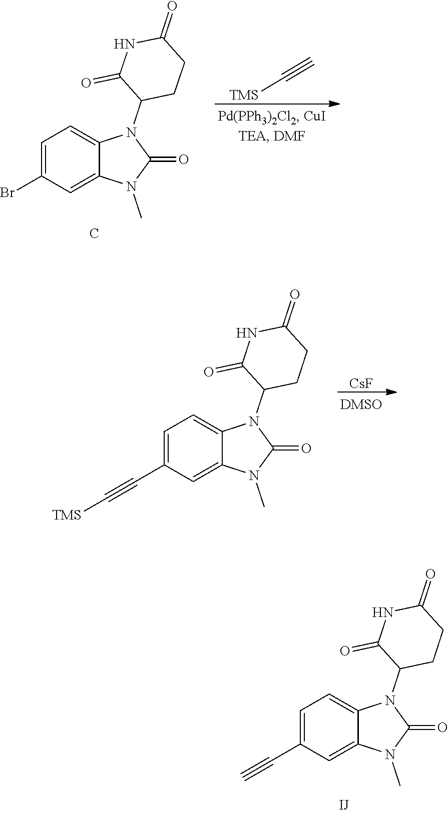

### Step 1—3-(3-Methyl-2-oxo-5-((trimethylsilyl)ethynyl)-2,3-dihydro-1H-benzo[d]imidazol-1-yl)piperidine-2,6-dione

To a solution of 3-(5-bromo-3-methyl-2-oxo-2,3-dihydro-1H-benzo[d]imidazol-1-yl)piperidine-2,6-dione (1.00 g, 2.96 mmol, Intermediate C) in DMF (10 mL) was added to Pd(PPh₃)₂Cl₂ (415 mg, 591 umol), CuI (56.3 mg, 295 umol) and TEA (2.99 g, 29.5 mmol, 4.12 mL). Then the mixture was degassed and purged with nitrogen three times at 25° C. Next, ethynyl(trimethyl)silane (871 mg, 8.87 mmol) was added, and the mixture was stirred at 85° C. for 1.5 h under microwave. On completion, the reaction mixture was quenched with sat. NH₄Cl (10 mL), and then extracted with EtOAc (10 mL×3). The combined organic layers were washed with brine (10 mL×3), dried over Na₂SO₄, filtered and concentrated under reduced pressure to give a residue. The residue was purified by flash silica gel chromatography (ISCO®; 80 g SepaFlash® Silica Flash Column, Eluent of 0˜100% Ethyl acetate/Petroleum ether gradient @100 mL/min) to give the title compound (0.91 g, 87% yield) as a yellow solid. ¹H NMR (400 MHz, DMSO-d6) δ=11.11 (s, 1H), 7.33 (d, J=1.2 Hz, 1H), 7.23-7.08 (m, 2H), 5.51-5.27 (m, 1H), 3.36-3.33 (m, 3H), 2.94-2.82 (m, 1H), 2.76-2.66 (m, 1H), 2.65-2.58 (m, 1H), 2.07-2.00 (m, 1H), 0.23 (s, 6H)

### Step 2—3-(5-ethynyl-3-methyl-2-oxo-2,3-dihydro-1H-benzo[d]imidazol-1-yl)piperidine-2,6-dione

To a solution of 3-(3-methyl-2-oxo-5-((trimethylsilyl)ethynyl)-2,3-dihydro-1H-benzo[d]imidazol-1-yl)piperidine-2,6-dione (8.20 g, 23.0 mmol) in DMSO (80 mL) was added to CsF (17.5 g, 115 mmol), then the mixture was stirred at 25° C. for 12 h. On completion, the reaction mixture was quenched with sat. NH₄Cl (80 mL), and then extracted with EtOAc (80 mL×3). The combined organic layers were washed with brine (80 mL×3), dried over Na₂SO₄, filtered and concentrated under reduced pressure to give the title compound (6.3 g) as a yellow solid. ¹H NMR (400 MHz, DMSO-d₆) δ=11.11 (s, 1H), 7.34 (s, 1H), 7.24-7.08 (m, 2H), 5.46-5.31 (m, 1H), 4.07 (s, 1H), 2.97-2.80 (m, 1H), 2.71-2.59 (m, 2H), 2.54 (s, 2H), 2.08-1.95 (m, 1H), 1.27-1.09 (m, 1H).

### Example 1.242. Preparation of tert-butyl 4-(3-oxocyclobutanecarbonyl)piperazine-1-carboxylate (Intermediate IK)

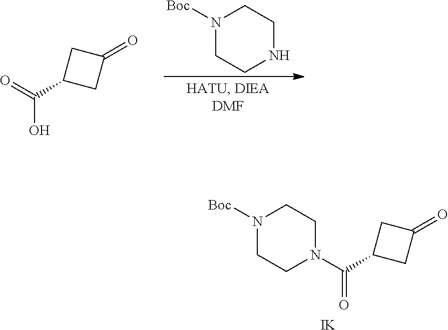

To a solution of 3-oxocyclobutanecarboxylic acid (5.00 g, 43.8 mmol, CAS #23761-23-1) in DMF (50 mL) was HATU (20.0 g, 52.6 mmol), DIEA (17.0 g, 132 mmol, 22.9 mL) and tert-butyl piperazine-1-carboxylate (8.16 g, 43.8 mmol, CAS #143238-38-4). Then the mixture was stirred at 25° C. for 12 h. On completion, the reaction mixture was quenched with NH₄Cl (50 mL) and extracted with EtOAC (60 mL×2). The combined organic layers were washed with brine (45 mL×3), dried over Na₂SO₄ and evaporated. The residue was column chromatography (Petroleum ether/Ethyl acetate=3:1-1:1) to give the title compound (12.3 g, 99% yield) as a white solid. ¹H NMR (400 MHz, DMSO-d₆) δ 3.50-3.42 (m, 5H), 3.32 (s, 3H), 3.27-3.21 (m, 4H), 2.69 (s, 1H), 1.41 (s, 9H).

### Example 1.243. Preparation of 4-(Prop-2-yn-1-yloxy)piperidine (Intermediate IL)

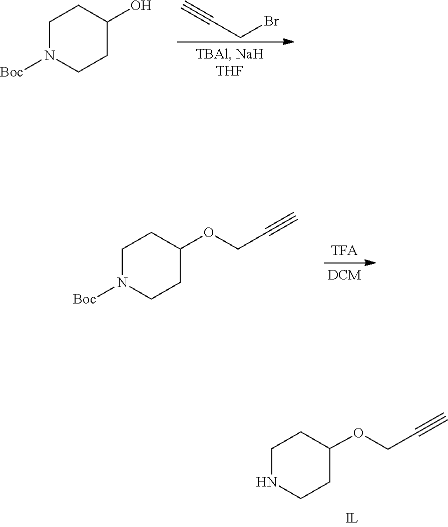

### Step 1—Tert-butyl 4-(prop-2-yn-1-yloxy)piperidine-1-carboxylate

To a solution of tert-butyl 4-hydroxypiperidine-1-carboxylate (20.0 g, 99.4 mmol) in THF (100 mL) was added NaH (4.77 g, 119 mmol, 60% dispersion in mineral oil) at 0° C. and stirred for 30 mins. Then TBAI (3.67 g, 9.94 mmol) and 3-bromoprop-1-yne (14.2 g, 119 mmol, 10.3 mL) was added and the mixture was stirred at 20° C. for 36 h. On completion, the reaction mixture was quenched with NH₄Cl (100 mL) at 0° C., and then extracted with EtOAc (100 mL×3). The combined organic layers were washed with sat. NaCl (100 mL×3), dried over Na₂SO₄, filtered and concentrated under reduced pressure to give a residue. The residue was purified by column chromatography (SiO₂, Petroleum ether/Ethyl acetate=10:1-7:1) to give the title compound (15.3 g, 64% yield) as a white solid. ¹H NMR (400 MHz, DMSO-d₆) δ 4.17 (d, J=2.0 Hz, 2H), 3.66-3.55 (m, 3H), 3.39-3.36 (m, 1H), 3.02 (br t, J=10.0 Hz, 2H), 1.84-1.71 (m, 2H), 1.39 (s, 9H), 1.34-1.26 (m, 2H).

### Step 2—4-(Prop-2-yn-1-yloxy)piperidine

To a solution of tert-butyl 4-prop-2-ynoxypiperidine-1-carboxylate (5.00 g, 20.9 mmol) in DCM (50 mL) and TFA (10 mL), then the mixture was stirred at 25° C. for 5 h. On completion, the mixture was concentrated to give the title compound (5 g) as a white solid. ¹H NMR (400 MHz, DMSO-d₆) δ 8.78-8.45 (m, 2H), 4.19 (d, J=2.4 Hz, 2H), 3.74 (td, J=4.0, 7.2 Hz, 1H), 3.44 (t, J=2.4 Hz, 1H), 3.15 (br d, J=6.8 Hz, 2H), 3.05-2.92 (m, 2H), 1.95 (dtd, J=3.2, 7.2, 13.6 Hz, 2H), 1.74-1.56 (m, 2H).

### Example 1.244. Preparation of tert-butyl 7-formyl-5-oxa-2-azaspiro[3.4]octane-2-carboxylate (Intermediate IM)

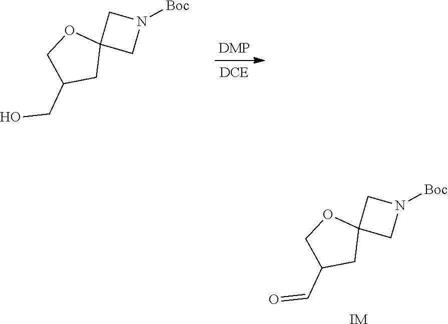

To a solution of tert-butyl 7-(hydroxymethyl)-5-oxa-2-azaspiro[3.4]octane-2-carboxylate (5.5 g, 22.6 mmol, CAS #1330764-06-1) in DCM (110 mL) was added DMP (14.3 g, 33.9 mmol) at 20° C. under nitrogen flow. Then the reaction was stirred at 20° C. for 10 h under nitrogen atmosphere. On completion, the reaction was poured into NaHCO₃ (sat. aq, 200 mL) and extracted with DCM (100 mL×2). The combined organic phase was washed with brine (70 mL×2), and dried over Na₂SO₄. After filtration, the filtrate was concentrated to give the title compound (9) as a yellow gum. ¹H NMR (400 MHz, DMSO-d₆) δ=9.59 (d, J=1.6 Hz, 1H), 4.06 (dd, J=4.4, 9.2 Hz, 1H), 3.91-3.82 (m, 3H), 3.79-3.67 (m, 2H), 3.26-3.15 (m, 1H), 2.40-2.32 (m, 1H), 2.23 (br d, J=9.2 Hz, 1H), 1.37 (s, 9H).

### Example 1.245. Preparation of N-((1R,2S)-2-fluorocyclopropyl)-6-(4-(5-formylpyridin-2-yl)indolin-1-yl)-8-(methylamino)imidazo[1,2-b]pyridazine-3-carboxamide (Intermediate IN)

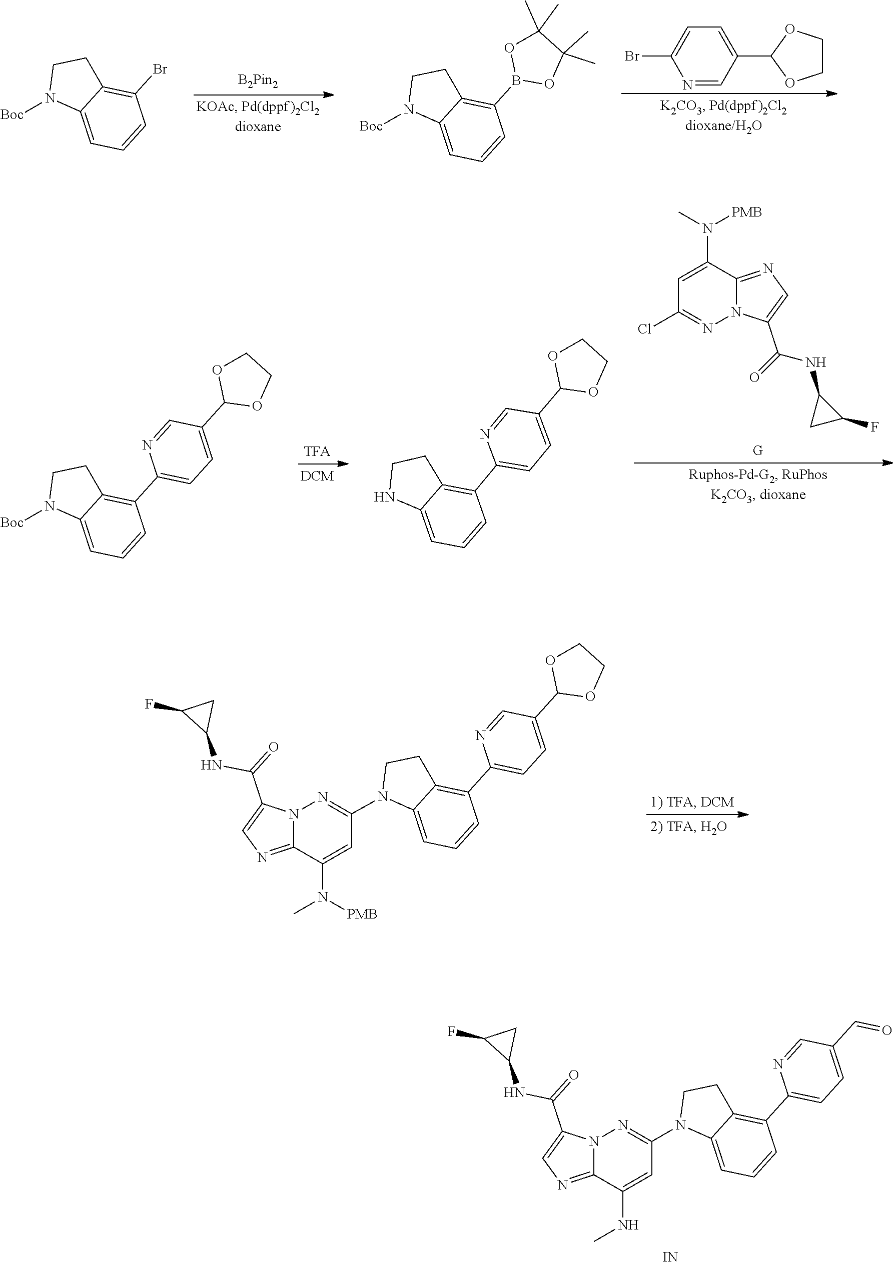

### Step 1—Tert-butyl 4-(4,4,5,5-tetramethyl-1,3,2-dioxaborolan-2-yl)indoline-1-carboxylate

To a stirred mixture of tert-butyl 4-bromo-2,3-dihydroindole-1-carboxylate (30.00 g, 100.6 mmol, CAS #885272-46-8) and B₂Pin₂ (38.32 g, 150.9 mmol) in dioxane (500 mL) was added KOAc (29.62 g, 301.8 mmol) and Pd(dppf)Cl₂·CH₂Cl₂ (8.20 g, 10.1 mmol) in portions at rt under nitrogen atmosphere. The resulting mixture was then stirred for 2 h at 80° C. under nitrogen atmosphere. On completion, the mixture was cooled to rt. The residue was purified by silica gel column chromatography, eluted with PE/EA (10:1˜3:1) to afford the title compound (30 g, 86% yield) as a yellow solid. LC/MS (ESI, m/z): [(M+H)]⁺=346.2.

### Step 2—Tert-butyl 4-(5-(1,3-dioxolan-2-yl)pyridin-2-yl)indoline-1-carboxylate

To a stirred mixture of tert-butyl 4-(4,4,5,5-tetramethyl-1,3,2-dioxaborolan-2-yl)-2,3-dihydroindole-1-carboxylate (43.80 g, 126.9 mmol) and 2-bromo-5-(1,3-dioxolan-2-yl)pyridine (35.02 g, 152.2 mmol) in dioxane (500 mL) and H₂O (80 mL) were added Pd(dppf)Cl₂CH₂Cl₂ (10.33 g, 12.69 mmol) and K₂CO₃ (35.07 g, 253.7 mmol) in portions at rt under nitrogen atmosphere. The resulting mixture was stirred for 2 h at 80° C. under nitrogen atmosphere. On completion, the mixture was cooled to rt. The residue was purified by silica gel column chromatography, eluted with PE/EA (5:1˜3:1) to afford the title compound (20.60 g, 44% yield) as a colorless oil. LC/MS (ESI, m/z): [(M+H)]⁺=369.2.

### Step 3—4-(5-(1,3-Dioxolan-2-yl)pyridin-2-yl)indoline

Into a 500 mL round-bottom flask were added tert-butyl 4-[5-(1,3-dioxolan-2-yl)pyridin-2-yl]-2,3-dihydroindole-1-carboxylate (20.00 g, 54.284 mmol) and TFA (100 mL) in DCM (100 mL) at rt. The resulting solution was stirred for 1 h at rt under nitrogen atmosphere. On completion, the reaction mixture was concentrated under reduced pressure. The residue was neutralized to pH 8 with saturated Na₂CO₃ (aq.). The resulting mixture was extracted with EtOAc (4×100 mL). The combined organic layers were washed with EtOAc (3×40 mL), and dried over anhydrous Na₂SO₄. After filtration, the filtrate was concentrated under reduced pressure to give the title compound (12.00 g, 82% yield) as a yellow solid. LC/MS (ESI, m/z): [(M+H)]⁺=269.1.

### Step 4—6-(4-(5-(1,3-Dioxolan-2-yl)pyridin-2-yl)indolin-1-yl)-N-((1R,2S)-2-fluorocyclopropyl)-8-((4-methoxybenzyl)(methyl)amino)imidazo[1,2-b]pyridazine-3-carboxamide

To a stirred mixture of 4-[5-(1,3-dioxolan-2-yl)pyridin-2-yl]-2,3-dihydro-1H-indole (11.00 g, 41.00 mmol) and 6-chloro-N-[(1R,2S)-2-fluorocyclopropyl]-8-{[(4-methoxyphenyl)methyl](methyl)amino}imidazo[1,2-b]pyridazine-3-carboxamide (19.87 g, 49.20 mmol, Intermediate G) in dioxane (100 mL) were added RuPhos-PdOMs-2nd G (3.43 g, 4.10 mmol), RuPhos (3.83 g, 8.20 mmol) and K₂CO₃ (17.00 g, 123.0 mmol) in portions at rt under nitrogen atmosphere. The mixture was then stirred for 4 h at 100° C. under nitrogen atmosphere. On completion, the mixture was cooled to rt. The residue was purified by silica gel column chromatography, eluted with PE/EA (5:1˜1:10) to afford the title compound (15.00 g, 58% yield) as a yellow solid. LC/MS (ESI, m/z): [(M+H)]⁺=636.3.

### Step 5—N-((1R,2S)-2-fluorocyclopropyl)-6-(4-(5-formylpyridin-2-yl)indolin-1-yl)-8-(methylamino)imidazo[1,2-b]pyridazine-3-carboxamide

A mixture of 6-{4-[5-(1,3-dioxolan-2-yl)pyridin-2-yl]-2,3-dihydroindol-1-yl}-N-[(1R,2S)-2-fluorocyclopropyl]-8-{[(4-methoxyphenyl)methyl](methyl)amino}imidazo[1,2-b]pyridazine-3-carboxamide (15.00 g, 23.60 mmol) and TFA (240 mL) in DCM (100 mL) was stirred for 2 h at rt under nitrogen atmosphere. On completion, the resulting mixture was concentrated under vacuum. Next, H₂O (100 mL, 2 mol/L TFA) was added and the mixture was stirred for 16 h at 50° C. under nitrogen atmosphere. On completion, the resulting mixture was concentrated under vacuum. The resulting mixture was filtered and the filter cake was washed with diethyl ether (5×20 mL). The filtrate cake was then dried under reduced pressure to give the title compound (8 g, 69% yield) as a yellow solid. ¹H NMR (400 MHz, DMSO-d₆) δ 10.18 (s, 1H), 9.21 (s, 1H), 8.89-8.84 (m, 1H), 8.39-8.32 (m, 1H), 8.05-7.99 (m, 1H), 7.97 (s, 1H), 7.85 (d, J=7.8 Hz, 1H), 7.75-7.67 (m, 1H), 7.46-7.39 (m, 1H), 7.39-7.31 (m, 1H), 6.03 (s, 1H), 4.91-4.70 (m, 1H), 4.23-4.14 (m, 2H), 3.54-3.46 (m, 2H), 3.09-3.01 (m, 1H), 2.98 (d, J=4.9 Hz, 3H), 1.30-1.16 (m, 1H), 1.05-0.90 (m, 1H); LC/MS (ESI, m/z): [(M+H)]⁺=472.3.

### Example 1.246. Preparation of 1-(2-Chloro-5-{2,7-diazaspiro[3.5]nonane-7-carbonyl}phenyl)-1,3-diazinane-2,4-dione (Intermediate IO)

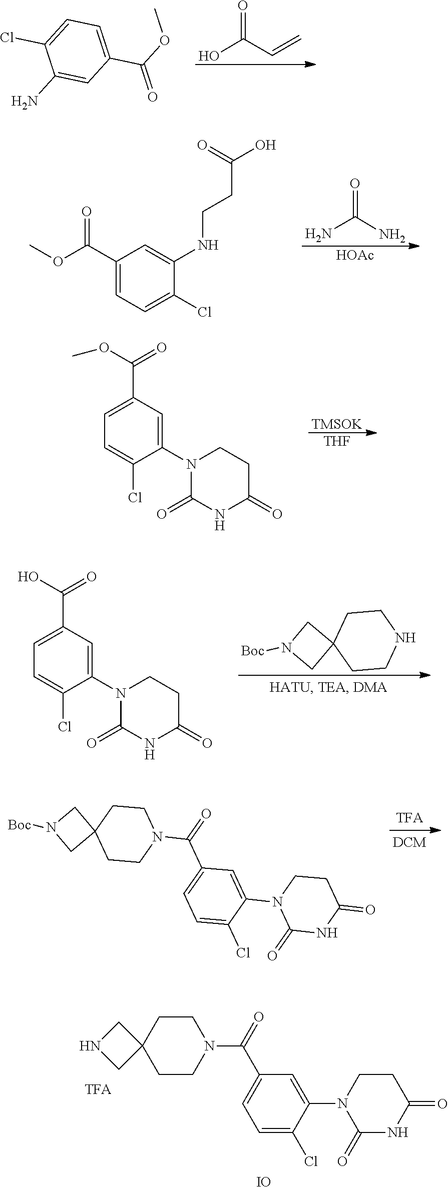

### Step 1—3-{[2-Chloro-5-(methoxycarbonyl)phenyl]amino}propanoic Acid

Into a 1000 mL round-bottom flask were added methyl 3-amino-4-chlorobenzoate (25 g, 140 mmol) and acrylic acid (200 mL) at rt. The resulting mixture was stirred for 4 h at 100° C. under nitrogen atmosphere. On completion, the reaction mixture was cooled to rt then diluted with water (2000 mL). The resulting mixture was extracted with EA (3×500 mL). The combined organic layers were washed with brine (3×200 mL), and dried over anhydrous Na₂SO₄. After filtration, the filtrate was concentrated under reduced pressure to give the title compound (25 g) as a yellow oil. LC/MS (ESI, m/z): [(M+H)]⁺=258.1.

### Step 2—Methyl 4-chloro-3-(2,4-dioxo-1,3-diazinan-1-yl)benzoate

To a stirred solution of 3-{[2-chloro-5-(methoxycarbonyl)phenyl]amino}propanoic acid (25 g, 97 mmol) in AcOH (200 mL) was added urea (40.79 g, 679.2 mmol) at rt under nitrogen atmosphere. The resulting mixture was stirred for 16 h at 110° C. under nitrogen atmosphere. On completion, the reaction mixture was cooled to rt and diluted with water (2000 mL). The resulting mixture was extracted with EA (3×500 mL). The combined organic layers were washed with brine (3×300 mL), and dried over anhydrous Na₂SO₄. After filtration, the filtrate was concentrated under reduced pressure. The residue was purified by trituration with MTBE (300 mL). The resulting mixture was filtered, and the filter cake was washed with MTBE (3×10 mL) was dried under reduced pressure to give the title compound (14 g, 51% yield) as a white solid. LC/MS (ESI, m/z): [(M+H)]⁺=283.1.

### Step 3—4-Chloro-3-(2,4-dioxo-1,3-diazinan-1-yl)benzoic Acid

To a stirred solution of methyl 4-chloro-3-(2,4-dioxo-1,3-diazinan-1-yl)benzoate (5 g, 20 mmol) in THF (100 mL) was added trimethyl(potassiooxy)silane (4.54 g, 35.4 mmol) at rt under nitrogen atmosphere. The resulting mixture was stirred for 2 h at rt under nitrogen atmosphere. On completion, the mixture was acidified to pH 4 with formic acid and the mixture was concentrated under reduced pressure. The residue was purified by reverse flash chromatography (column: C₁₈ silica gel; mobile phase, MeCN in Water (0.1% FA), 15% to 45% gradient in 30 min; detector, UV 254 nm, desired fractions were collected at 23% B) and concentrated under reduced pressure to afford the title compound (2 g, 42% yield) as a white solid. LC/MS (ESI, m/z): [(M+H)]⁺=269.0.

### Step 4—Tert-butyl 7-[4-chloro-3-(2,4-dioxo-1,3-diazinan-1-yl)benzoyl]-2,7-diazaspiro[3.5]nonane-2-carboxylate

To a stirred solution of 4-chloro-3-(2,4-dioxo-1,3-diazinan-1-yl)benzoic acid (1 g, 4 mmol) and tert-butyl 2,7-diazaspiro[3.5]nonane-2-carboxylate (0.84 g, 3.7 mmol) in DMA (10 mL) was added TEA (0.75 g, 7.4 mmol) dropwise at rt under nitrogen atmosphere. To the above mixture was added HATU (1.70 g, 4.466 mmol) at 0° C. Then the resulting mixture was stirred for additional 2 h at rt. On completion, the reaction mixture was concentrated under reduced pressure. The residue was purified by reverse flash chromatography (column: C₁₈ silica gel; mobile phase, MeCN in Water (0.1% FA), 15% to 45% gradient in 30 min; detector, UV 254 nm, desired fractions were collected at 31% B) concentrated under reduced pressure to afford the title compound (1.7 g, 96% yield) as a yellow solid. LC/MS (ESI, m/z): [(M+H)]⁺=477.2.

### Step 5—1-(2-Chloro-5-{2,7-diazaspiro[3.5]nonane-7-carbonyl}phenyl)-1,3-diazinane-2,4-dione Trifluoroacetate

To a stirred solution of tert-butyl 7-[4-chloro-3-(2,4-dioxo-1,3-diazinan-1-yl)benzoyl]-2,7-diazaspiro[3.5]nonane-2-carboxylate (500 mg, 1 mmol) in DCM (10 mL) was added TFA (2 mL) dropwise at rt under nitrogen atmosphere. The resulting mixture was stirred for 3 h at room temperature under nitrogen atmosphere. On completion, the mixture was concentrated under reduced pressure. The residue was purified by trituration with diethyl ether (30 mL) to afford the title compound (400 mg, 80% yield) as a yellow solid. LC/MS (ESI, m/z): [(M+H)]⁺=377.1.

### Example 1.247. Preparation of 1-{5-[4-(azetidin-3-yl)piperazine-1-carbonyl]-2-chlorophenyl}-1,3-diazinane-2,4-dione (Intermediate IP)

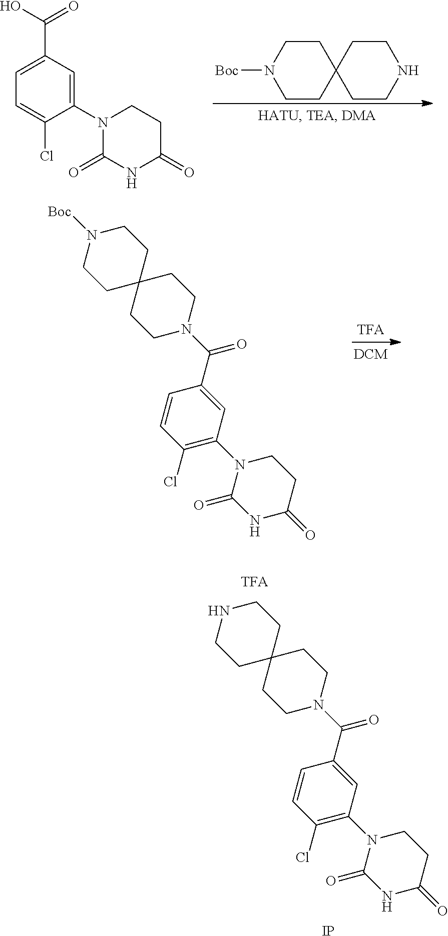

### Step 1—Tert-butyl 9-[4-chloro-3-(2,4-dioxo-1,3-diazinan-1-yl)benzoyl]-3,9-diazaspiro[5.5]undecane-3-carboxylate

To a stirred solution of 4-chloro-3-(2,4-dioxo-1,3-diazinan-1-yl)benzoic acid (500 mg, 2 mmol, synthesized via Steps 1-3 of Intermediate IO) and tert-butyl 3,9-diazaspiro[5.5]undecane-3-carboxylate (473.43 mg, 1.861 mmol, CAS #173405-78-2) in DMA (10 mL) were added HATU (1061.52 mg, 2.792 mmol) and TEA (1.03 ml, 7.44 mmol) at rt under nitrogen atmosphere. The resulting mixture was stirred for 1 h at rt under nitrogen atmosphere. On completion, the mixture was concentrated under vacuum. The residue was purified by reverse flash chromatography (column: C18 silica gel; mobile phase A: water (10 mmol/L FA), mobile phase B: ACN, 30% to 50% gradient in 30 min; detector, UV 254 nm; the fractions were collected at 40%) and concentrated under reduced pressure to afford the title compound (447 mg, 48%) as a yellow solid. LC/MS (ESI, m/z): [(M−56+H)]⁺=449.1.

### Step 2—1-{5-[4-(Azetidin-3-yl)piperazine-1-carbonyl]-2-chlorophenyl}-1,3-diazinane-2,4-dione Trifluoroacetate

To a stirred solution of tert-butyl 3-{4-[4-chloro-3-(2,4-dioxo-1,3-diazinan-1-yl)benzoyl]piperazin-1-yl}azetidine-1-carboxylate (400 mg, 0.813 mmol) in DCM (7 mL) was added TFA (2 mL) dropwise at rt. The resulting mixture was stirred for 1 h at rt under nitrogen atmosphere. On completion, the reaction mixture was concentrated under reduced pressure. The residue was purified by trituration with Et₂O (5 mL) and filtered to afford the title compound (385 mg) as a white solid. LC/MS (ESI, m/z): [(M+H)]⁺=405.2.

### Example 1.248. Preparation of 3-{4-[4-(azetidin-3-yl)piperazin-1-yl]-3-methyl-2-oxo-1,3-benzodiazol-1-yl}piperidine-2,6-dione (Intermediate 10)

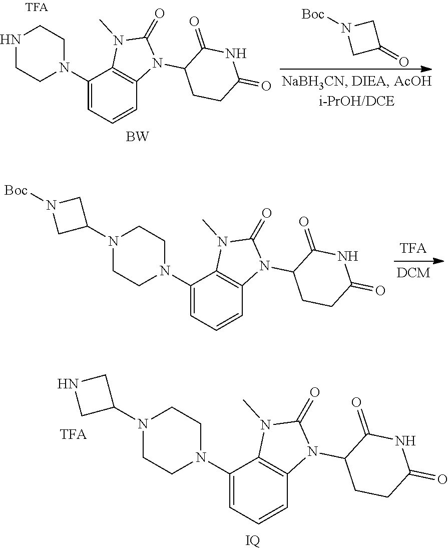

### Step 1—Tert-butyl 3-{4-[1-(2,6-dioxopiperidin-3-yl)-3-methyl-2-oxo-1,3-benzodiazol-4-yl]piperazin-1-yl}azetidine-1-carboxylate

A solution of 3-[3-methyl-2-oxo-4-(piperazin-1-yl)-1,3-benzodiazol-1-yl]piperidine-2,6-dione trifluoroacetate (3.7 g, 8.4 mmol, Intermediate BW) in i-PrOH (25 mL) and DCE (25 mL) was treated with DIEA (3.25 g, 25.146 mmol) for 10 min at rt under nitrogen atmosphere followed by the addition of tert-butyl 3-oxoazetidine-1-carboxylate (2.15 g, 12.6 mmol) and AcOH (0.25 g, 4.2 mmol) for 30 min at rt. To the above mixture was added NaBH₃CN (1.58 g, 25.2 mmol) at rt. The resulting mixture was stirred for additional 16 h at rt. On completion, the resulting mixture was concentrated under reduced pressure. The residue was purified by reverse flash chromatography (column: C18 silica gel; mobile phase, ACN in Water (0.1% TFA), 20% to 40% gradient in 20 min; detector, UV 254 nm) and the resulting mixture was concentrated under reduced pressure to give the title compound (2.27 g, 54% yield) as a yellow oil. LC/MS (ESI, m/z): [(M+1)]⁺=499.3.

### Step 2—3-{4-[4-(Azetidin-3-yl)piperazin-1-yl]-3-methyl-2-oxo-1,3-benzodiazol-1-yl}piperidine-2,6-dione Trifluoroacetate

A solution of tert-butyl 3-{4-[1-(2,6-dioxopiperidin-3-yl)-3-methyl-2-oxo-1,3-benzodiazol-4-yl]piperazin-1-yl}azetidine-1-carboxylate (2.27 g, 4.55 mmol) and TFA (3 mL) in DCM (15 mL) was stirred for 2 h at rt under nitrogen atmosphere. On completion, the resulting mixture was concentrated under reduced pressure. The residue was purified by trituration with diethyl ether (40 mL). The precipitated solids were collected by filtration and washed with diethyl ether (3×10 mL) and dried under vacuum to give the title compound as a yellow oil. LC/MS (ESI, m/z): [(M+1)]⁺=399.2.

### Example 1.249. Preparation of 3-(3-methyl-2-oxo-4-{4-[1-(piperidin-4-yl)azetidin-3-yl]piperazin-1-yl}-1,3-benzodiazol-1-yl)piperidine-2,6-dione (Intermediate IR)

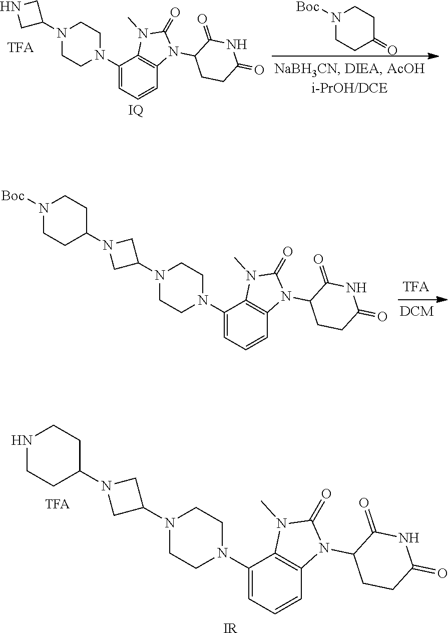

### Step 1—Tert-butyl 4-(3-{4-[1-(2,6-dioxopiperidin-3-yl)-3-methyl-2-oxo-1,3-benzodiazol-4-yl]piperazin-1-yl}azetidin-1-yl)piperidine-1-carboxylate

A solution of 3-{4-[4-(azetidin-3-yl)piperazin-1-yl]-3-methyl-2-oxo-1,3-benzodiazol-1-yl}piperidine-2,6-dione trifluoroacetate (2.18 g, 4.39 mmol, Intermediate IQ) in i-PrOH (10 mL) and DCE (10 mL) was treated with DIEA (1.70 g, 13.2 mmol) for 10 min at rt under nitrogen atmosphere. Next, tert-butyl 4-oxopiperidine-1-carboxylate (1.31 g, 6.59 mmol) and AcOH (0.13 g, 2.196 mmol) was added and the mixture was stirred for 30 min at rt. To the above mixture was added NaBH₃CN (0.83 g, 13.173 mmol) at rt and the mixture was stirred for additional 16 h at rt. On completion, the reaction mixture was concentrated under reduced pressure. The residue was purified by reverse flash chromatography (column: C18 silica gel; mobile phase, ACN in Water (0.1% TFA), 20% to 40% gradient in 20 min; detector, UV 254 nm) and the resulting mixture was concentrated under reduced pressure to give the title compound (800 mg, 31% yield) as a yellow solid. LC/MS (ESI, m/z): [(M+1)]⁺=582.4.

### Step 2—3-(3-Methyl-2-oxo-4-{4-[1-(piperidin-4-yl)azetidin-3-yl]piperazin-1-yl}-1,3-benzodiazol-1-yl)piperidine-2,6-dione Trifluoroacetate

To a stirred solution of tert-butyl 4-(3-{4-[1-(2,6-dioxopiperidin-3-yl)-3-methyl-2-oxo-1,3-benzodiazol-4-yl]piperazin-1-yl}azetidin-1-yl)piperidine-1-carboxylate (600 mg, 1 mmol) in DCM (10 mL) was added TFA (2 mL) at rt under nitrogen atmosphere. The resulting mixture was stirred for 16 h at rt under nitrogen atmosphere. On completion, the mixture was concentrated under reduced pressure. The residue was purified by trituration with diethyl ether (30 mL). The precipitated solids were collected by filtration and washed with diethyl ether (3×10 mL) and dried under vacuum to give the title compound (700 mg) as a white solid. LC/MS (ESI, m/z): [(M+1)]⁺=482.4.

### Example 1.250. Preparation of N-[(1R,2S)-2-fluorocyclopropyl]-6-{[3-(5-formylpyridin-2-yl)-2-methoxyphenyl]amino}-8-(methylamino)imidazo[1,2-b]pyridazine-3-carboxamide (Intermediate IS)

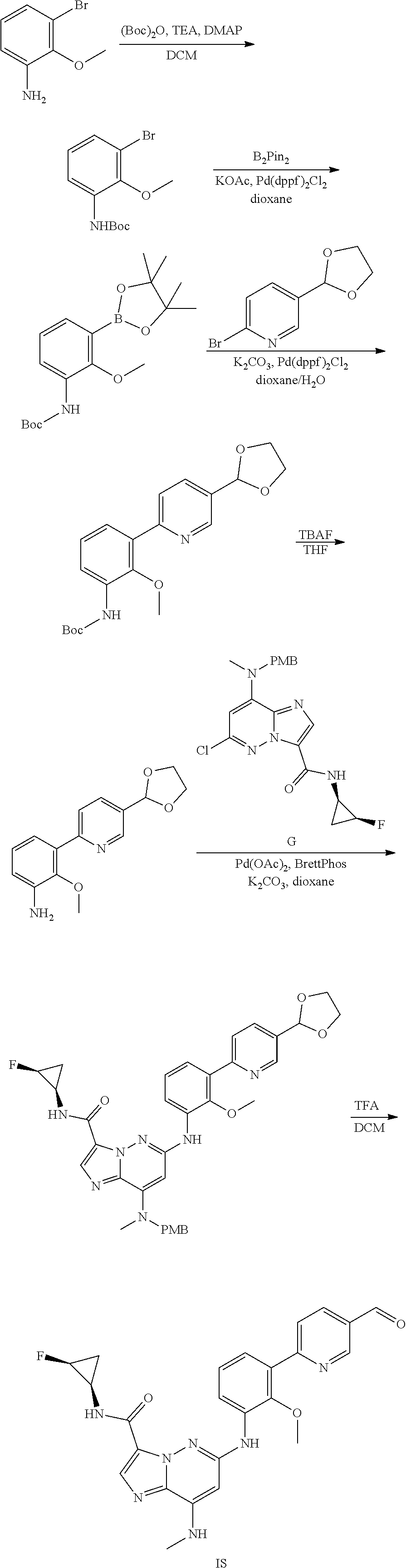

### Step 1—Tert-butyl N-(3-bromo-2-methoxyphenyl)carbamate

A solution of 3-bromo-2-methoxyaniline (10 g, 50 mmol) and Boc₂O (16.20 g, 74.24 mmol) in EtOH (150 mL) was stirred for 16 h at rt under nitrogen atmosphere. On completion, the reaction mixture was concentrated under reduced pressure. The residue was purified by silica gel column chromatography, eluted with PE/EA (10:1), to afford the title compound (14.5 g, 97% yield) as a yellow oil. LC/MS (ESI, m/z): [(M+1)]⁺=302.2.

### Step 2—Tert-butyl N-[2-methoxy-3-(4,4,5,5-tetramethyl-1,3,2-dioxaborolan-2-yl)phenyl]carbamate

To a stirred solution of tert-butyl N-(3-bromo-2-methoxyphenyl)carbamate (11 g, 36 mmol) and AcOK (7.15 g, 72.8 mmol) in dioxane (150 mL) were added bis(pinacolato)diboron (13.87 g, 54.61 mmol) and Pd(dppf)Cl₂·CH₂Cl₂ (1.48 g, 1.82 mmol) at rt under nitrogen atmosphere. Then the reaction mixture was stirred for 16 h at 80° C. under nitrogen atmosphere. On completion, the reaction mixture was cooled to rt and was concentrated under reduced pressure. The residue was purified by silica gel column chromatography, eluted with PE/EA (12:1), to afford the title compound (10.48 g) as a yellow oil. LC/MS (ESI, m/z): [(M+1)]⁺=350.2.

### Step 3—Tert-butyl N-{3-[5-(1,3-dioxolan-2-yl)pyridin-2-yl]-2-methoxyphenyl}carbamate

To a stirred solution of tert-butyl N-[2-methoxy-3-(4,4,5,5-tetramethyl-1,3,2-dioxaborolan-2-yl)phenyl]carbamate (10 g, 30 mmol) and 2-bromo-5-(1,3-dioxolan-2-yl)pyridine (6.59 g, 28.6 mmol, synthesized via Step 1 of Intermediate BC) in dioxane (100 mL) and H₂O (20 mL) were added K₂CO₃ (7.91 g, 57.3 mmol) and Pd(dppf)Cl₂CH₂Cl₂ (2.33 g, 2.86 mmol) at rt under nitrogen atmosphere. The resulting mixture was stirred for 16 h at 80° C. under nitrogen atmosphere. On completion, the mixture was cooled to rt and was concentrated under reduced pressure. The residue was purified by reverse flash chromatography (column: C18 silica gel; mobile phase, ACN in Water (0.1% FA), 40% to 60% gradient in 20 min; detector, UV 254 nm; desired fraction were collected at 53% B) and concentrated under reduced pressure to afford the title compound (4.6 g, 43% yield) as a black oil. LC/MS (ESI, m/z): [(M+1)]⁺=373.1.

### Step 4—3-[5-(1,3-Dioxolan-2-yl)pyridin-2-yl]-2-methoxyaniline

To a stirred solution of tert-butyl N-{3-[5-(1,3-dioxolan-2-yl)pyridin-2-yl]-2-methoxyphenyl}carbamate (4.5 g, 12 mmol) in THF (50 mL) was added TBAF (50 mL, 0.5 mol/L, THF solution) at rt under nitrogen atmosphere. The resulting mixture was stirred for 16 h at 50° C. under nitrogen atmosphere. On completion, the mixture was cooled to rt and was concentrated under reduced pressure. The residue was purified by silica gel column chromatography, eluted with PE/EA (3:1), to afford the title compound (2.5 g, 76% yield) as a yellow solid. LC/MS (ESI, m/z): [(M+1)]⁺=273.1.

### Step 5—6-({3-[5-(1,3-Dioxolan-2-yl)pyridin-2-yl]-2-methoxyphenyl}amino)-N-[(1R,2S)-2-fluorocyclopropyl]-8-{[(4-methoxyphenyl)methyl](methyl)amino}imidazo[1,2-b]pyridazine-3-carboxamide

To a stirred solution of 3-[5-(1,3-dioxolan-2-yl)pyridin-2-yl]-2-methoxyaniline (2.5 g, 9.2 mmol) and K₂CO₃ (2.54 g, 18.4 mmol) in dioxane (50 mL) were added 6-chloro-N-[(1R,2S)-2-fluorocyclopropyl]-8-{[(4-methoxyphenyl)methyl](methyl)amino}imidazo[1,2-b]pyridazine-3-carboxamide (3.71 g, 9.18 mmol, Intermediate G), BrettPhos (9.86 mg, 0.018 mmol) and Pd(OAc)₂ (0.21 g, 0.92 mmol) at rt under nitrogen atmosphere. The resulting mixture was then stirred for 16 h at 100° C. under nitrogen atmosphere. On completion, the mixture was cooled to rt and was concentrated under reduced pressure. The residue was purified by reverse flash chromatography (column: C18 silica gel; mobile phase, ACN in Water (10 mmol/L NH₄HCO₃), 40% to 60% gradient in 20 min; detector, UV 254 nm; desired fraction were collected at 52% B) and concentrated under reduced pressure to afford the title compound (3.27 g, 56% yield) as a yellow oil. LC/MS (ESI, m/z): [(M+1)]⁺=640.3.

### Step 6—N-[(1R,2S)-2-fluorocyclopropyl]-6-{[3-(5-formylpyridin-2-yl)-2-methoxyphenyl]amino}-8-(methylamino)imidazo[1,2-b]pyridazine-3-carboxamide

To a stirred solution of 6-({3-[5-(1,3-dioxolan-2-yl)pyridin-2-yl]-2-methoxyphenyl}amino)-N-[(1R,2S)-2-fluorocyclopropyl]-8-{[(4-methoxyphenyl)methyl](methyl)amino}imidazo[1,2-b]pyridazine-3-carboxamide (3.3 g, 5.2 mmol) in DCM (40 mL) was added TFA (8 mL) at rt under nitrogen atmosphere. The resulting mixture was stirred for 2 h at rt under nitrogen atmosphere. On completion, the reaction was concentrated under reduced pressure. Then TFA (2 mol/L, 20 mL) was added to the residue and the resulting mixture was stirred for additional 16 h at 50° C. On completion, the mixture was cooled to rt and concentrated under reduced pressure. The residue was purified by trituration with diethyl ether (50 mL). The precipitated solids were collected by filtration, washed with diethyl ether (5×10 mL) and dried under reduced pressure to give the title compound (2.5 g) as a yellow solid. LC/MS (ESI, m/z): [(M+1)]⁺=476.2.

### Example 1.251. Preparation of 3-{3-methyl-2-oxo-5-[(1r,4r)-4-(piperazin-1-yl)cyclohexyl]-1,3-benzodiazol-1-yl}piperidine-2,6-dione (Intermediate IT)

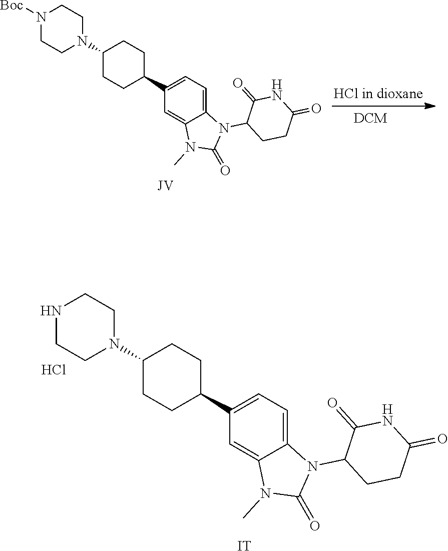 [SMILES: Cn1c(=O)n(C2CCC(=O)NC2=O)c2ccc([C@H]3CC[C@H](N4CCN(C(=O)OC(C)(C)C)CC4)CC3)cc21 ; Cl ; Cn1c(=O)n(C2CCC(=O)NC2=O)c2ccc([C@H]3CC[C@H](N4CCNCC4)CC3)cc21 ; "IT"]

To a stirred solution of tert-butyl 4-[(1r,4r)-4-[1-(2,6-dioxopiperidin-3-yl)-3-methyl-2-oxo-1,3-benzodiazol-5-yl]cyclohexyl]piperazine-1-carboxylate (600 mg, 1 mmol, Intermediate JV) in DCM (10 mL) was added 4 M HCl (gas) in 1,4-dioxane (2 mL) dropwise at rt under nitrogen atmosphere. The resulting mixture was stirred for 2 h at rt under nitrogen atmosphere. On completion, the reaction mixture was concentrated under reduced pressure. The residue was purified by trituration with ethyl ether (20 mL) and filtered and dried under reduced pressure to afford the title compound (500 mg) as a light grey solid. LC/MS (ESI, m/z): [(M+H)]⁺=426.3.

### Example 1.252. Preparation of Ethyl 5,7-dichloropyrazolo[1,5-a]pyrimidine-3-carboxylate (Intermediate IU)

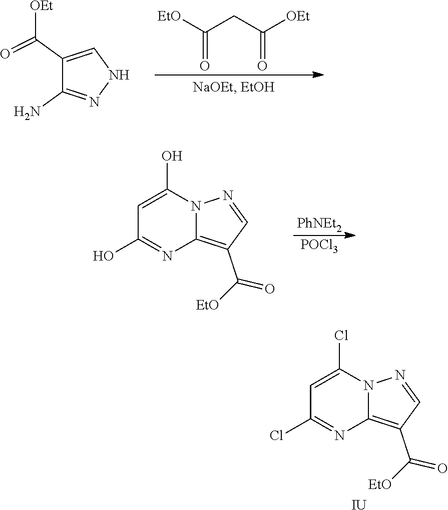

### Step 1—Ethyl 5,7-dihydroxypyrazolo[1,5-a]pyrimidine-3-carboxylate

To a stirred solution of ethyl 3-amino-1H-pyrazole-4-carboxylate (5 g, 30 mmol) and diethyl malonate (10.32 g, 64.45 mmol) in EtOH (25 mL) was added EtONa (7.58 mL, 96.7 mmol, 30 wt %) dropwise at rt under nitrogen atmosphere. The resulting mixture was stirred for 16 h at 80° C. under nitrogen atmosphere. On completion, the reaction was cooled to rt and concentrated under reduced pressure. The residue was purified by reverse phase flash chromatography (Column: WelFlash™ C18-I, 20-40 μm, 330 g; Eluent A: Water (plus 5 mmol/L NH₄HCO₃); Eluent B: ACN; Gradient: 15%-45% B in 35 min; Flow rate: 80 mL/min; Detector: 220/254 nm; desired fractions were collected at 21% B) and concentrated under reduced pressure to afford the title compound (1.32 g, 18% yield) as an off-white solid. LC/MS (ESI, m/z): [(M+H)]⁺=224.1.

### Step 2—Ethyl 5,7-dichloropyrazolo[1,5-a]pyrimidine-3-carboxylate

To a stirred solution of ethyl 5,7-dihydroxypyrazolo[1,5-a]pyrimidine-3-carboxylate (1.9 g, 8.5 mmol) in POCl₃ (5.17 mL) was added N,N-diethylaniline (1.91 g, 12.8 mmol) at rt under nitrogen atmosphere. The resulting mixture was stirred for 16 h at 80° C. under nitrogen atmosphere. On completion, the reaction was cooled to rt and concentrated under reduced pressure to obtained residue which was transferred into ice cold water (30 mL) and extracted with dichloromethane (3×20 mL). The combined organic layer was washed with saturated aq. sodium bicarbonate (30 mL), followed by brine (30 mL), and dried over sodium sulphate. After filtration, the filtrate was concentrated under reduced pressure to obtain crude. The crude was further purified by column chromatography, eluted in 20% ethyl acetate in Petroleum ether, to obtain the title compound (830 mg, 38% yield) as a yellow solid. LC/MS (ESI, m/z): [(M+H)]⁺=260.0.

### Example 1.253. Preparation of 5-chloro-N-[(1R,2S)-2-fluorocyclopropyl]-7-{[(4-methoxyphenyl)methyl](methyl)amino}pyrazolo[1,5-a]pyrimidine-3-carboxamide (Intermediate IV)

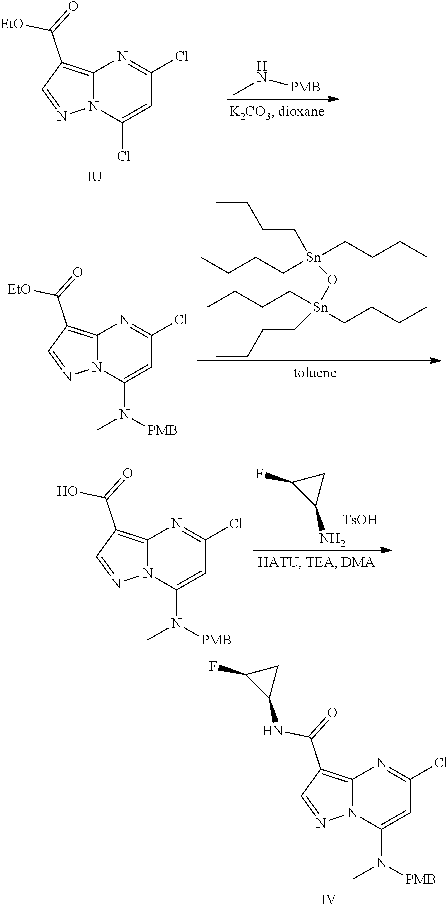

### Step 1—Ethyl 5-chloro-7-{[(4-methoxyphenyl)methyl](methyl)amino}pyrazolo[1,5-a]pyrimidine-3-carboxylate

To a stirred solution of ethyl 5,7-dichloropyrazolo[1,5-a]pyrimidine-3-carboxylate (12 g, 46 mmol, Intermediate IU) and [(4-methoxyphenyl)methyl](methyl)amine (8.37 g, 55.4 mmol) in dioxane (200 mL) was added K₂CO₃ (12.75 g, 92.28 mmol) at rt under nitrogen atmosphere. The resulting mixture was stirred for 16 h at 80° C. under nitrogen atmosphere. On completion, the reaction mixture was cooled to rt and filtered. The filter cake was washed with 1,4-dioxane (3×10 mL) and the filtrate was concentrated under reduced pressure. The residue was purified by reverse flash chromatography (column, C18 silica gel; mobile phase, MeCN in Water (0.1% FA), 60% to 90% gradient in 30 min; detector, UV 254 nm) to afford the title compound (12 g, 69% yield) as a yellow solid. LC/MS (ESI, m/z): [(M+H)]⁺=375.2.

### Step 2—5-Chloro-7-{[(4-methoxyphenyl)methyl](methyl)amino}pyrazolo[1,5-a]pyrimidine-3-carboxylic Acid

To a stirred solution of ethyl 5-chloro-7-{[(4-methoxyphenyl)methyl](methyl)amino}pyrazolo[1,5-a]pyrimidine-3-carboxylate (12 g, 32 mmol) in toluene (250 mL) was added tributyl[(tributylstannyl)oxy]stannane (76.34 g, 128.06 mmol) at rt under nitrogen atmosphere. The resulting mixture was stirred for 16 h at 120° C. under nitrogen atmosphere. On completion, the reaction mixture was cooled to rt and concentrated under reduced pressure. The residue was purified by silica gel column chromatography, eluted with CH₂Cl₂/MeOH (10:1), to afford the title compound (6 g, 54% yield) as a yellow oil. LC/MS (ESI, m/z): [(M+H)]⁺=347.2.

### Step 3—5-Chloro-N-[(1R,2S)-2-fluorocyclopropyl]-7-{[(4-methoxyphenyl)methyl](methyl)amino}pyrazolo[1,5-a]pyrimidine-3-carboxamide

To a stirred solution of 5-chloro-7-{[(4-methoxyphenyl)methyl](methyl)amino}pyrazolo[1,5-a]pyrimidine-3-carboxylic acid (5.5 g, 16 mmol) and HATU (7.24 g, 19.0 mmol) in DMA (50 mL) were added (1R,2S)-2-fluorocyclopropan-1-amine 4-methylbenzenesulfonate (4.7 g, 19.033 mmol) and TEA (6.61 mL, 47.583 mmol) at rt under nitrogen atmosphere. The resulting mixture was stirred for 1 h at rt under nitrogen atmosphere. On completion, the mixture was concentrated under reduced pressure. The residue was purified by reverse flash chromatography (column, C18 silica gel; mobile phase, MeCN in Water (10 mmol/L NH₄HCO₃), 45% to 75% gradient in 30 min; detector, UV 254 nm) to afford the title compound (5.6 g, 87% yield) as a yellow solid. LC/MS (ESI, m/z): [(M+H)]⁺=404.1.

### Example 1.254. Preparation of N-[(1R,2S)-2-fluorocyclopropyl]-5-{[3-(5-formylpyridin-2-yl)phenyl]amino}-7-(methylamino)pyrazolo[1,5-a]pyrimidine-3-carboxamide (Intermediate IW)

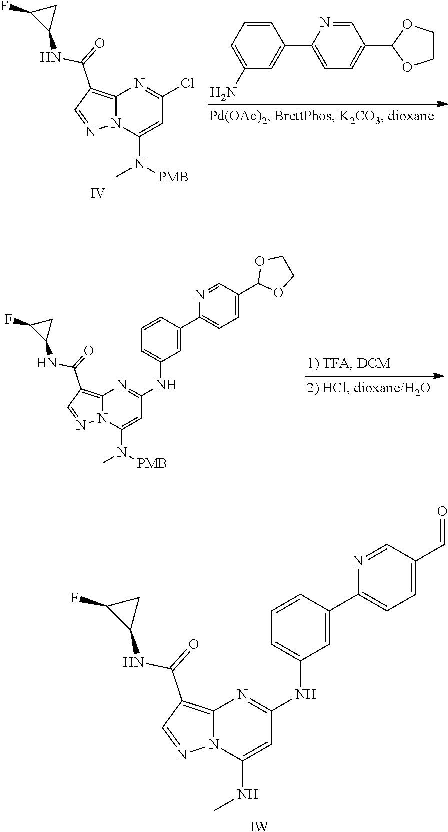

### Step 1—5-({3-[5-(1,3-dioxolan-2-yl)pyridin-2-yl]phenyl}amino)-N-[(1R,2S)-2-fluorocyclopropyl]-7-{[(4-methoxyphenyl)methyl](methyl)amino}pyrazolo[1,5-a]pyrimidine-3-carboxamide

To a stirred solution of 5-chloro-N-[(1R,2S)-2-fluorocyclopropyl]-7-{[(4-methoxyphenyl)methyl](methyl)amino}pyrazolo[1,5-a]pyrimidine-3-carboxamide (2 g, 5 mmol, Intermediate IV) and 3-[5-(1,3-dioxolan-2-yl)pyridin-2-yl]aniline (1.20 g, 4.95 mmol, Intermediate BC) in dioxane (20 mL) and H₂O (4 mL) was added K₂CO₃ (1.37 g, 9.91 mmol) at rt under nitrogen atmosphere. To the above mixture were added BrettPhos (265.84 mg, 0.495 mmol) and Pd(OAc)₂ (111.19 mg, 0.495 mmol) at rt. The resulting mixture was then stirred for 16 h at 90° C. under nitrogen atmosphere. On completion, the reaction was cooled to rt and filtered. The filter cake was washed with 1,4-dioxane (3×10 mL) and the filtrate was concentrated under reduced pressure. The residue was purified by silica gel column chromatography, eluted with DCM/EA (1:1), to afford the title compound (1.2 g, 40% yield) as a yellow solid. LC/MS (ESI, m/z): [(M+H)]⁺=610.3.

### Step 2—N-[(1R,2S)-2-fluorocyclopropyl]-5-{[3-(5-formylpyridin-2-yl)phenyl]amino}-7-(methylamino)pyrazolo[1,5-a]pyrimidine-3-carboxamide

To a stirred solution of 5-({3-[5-(1,3-dioxolan-2-yl)pyridin-2-yl]phenyl}amino)-N-[(1R,2S)-2-fluorocyclopropyl]-7-{[(4-methoxyphenyl)methyl](methyl)amino}pyrazolo[1,5-a]pyrimidine-3-carboxamide (1.2 g, 2.0 mmol) in DCM (10 mL) was added TFA (2 mL) at rt under nitrogen atmosphere. The resulting mixture was stirred for 2 h at rt under nitrogen atmosphere. On completion, the reaction mixture was concentrated under reduced pressure. The residue was dissolved in H₂O (5 mL) and dioxane (5 mL). Then, to the mixture was added 1 N HCl (5 mL) dropwise at rt. The resulting mixture was stirred for 4 h at 60° C. under nitrogen atmosphere. On completion, the reaction mixture was cooled to rt and concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 30%-60% B in 30 min; Flow rate: 80 mL/min; Detector: 254 nm; desired fractions were collected at 42% B) and concentrated under reduced pressure to afford the title compound (600 mg, 68% yield) as a brown solid. LC/MS (ESI, m/z): [(M+H)]⁺=446.2.

### Example 1.255. 6-{3-[(3-{[(1R,2S)-2-fluorocyclopropyl]carbamoyl}-8-(methylamino)imidazo[1,2-b]pyridazin-6-yl)amino]phenyl}pyridine-3-carboxylic Acid (Intermediate IX)

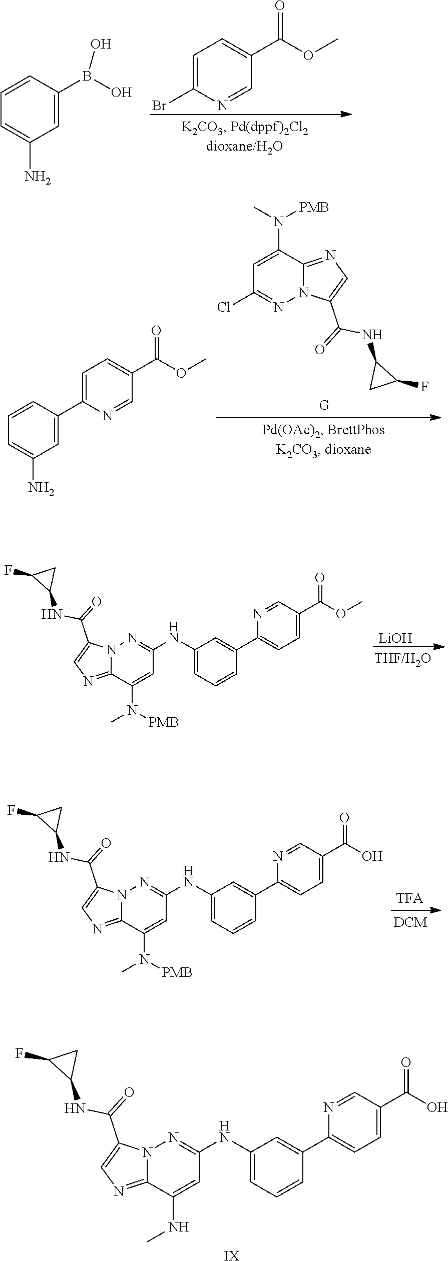

### Step 1—Methyl 6-(3-aminophenyl)pyridine-3-carboxylate

To a stirred solution of M-aminophenylboronic acid (10 g, 70 mmol) and methyl 6-bromopyridine-3-carboxylate (18.93 g, 87.62 mmol) in dioxane (100 mL) and H₂O (20 mL) were added Pd(dppf)Cl₂ CH₂Cl₂ (5.95 g, 7.30 mmol) and K₂CO₃ (30.27 g, 219.1 mmol) in portions at rt under nitrogen atmosphere. The resulting mixture was stirred for 4 h at 80° C. under nitrogen atmosphere. On completion, the reaction mixture was cooled to rt and concentrated under reduced pressure. The residue was purified by silica gel column chromatography, eluted with CH₂Cl₂/MeOH (10:1) to afford the title compound (8.3 g, 50% yield) as a yellow solid. LC/MS (ESI, m/z): [(M+H)]⁺=229.1.

### Step 2—6-{3-[(3-{[(1R,2S)-2-fluorocyclopropyl]carbamoyl}-8-{[(4-methoxyphenyl)methyl](methyl)amino}imidazo[1,2-b]pyridazin-6-yl)amino]phenyl}pyridine-3-carboxylate

To a stirred solution of methyl 6-(3-aminophenyl)pyridine-3-carboxylate (2 g, 9 mmol) and 6-chloro-N-[(1R,2S)-2-fluorocyclopropyl]-8-{[(4-methoxyphenyl)methyl](methyl)amino}imidazo[1,2-b]pyridazine-3-carboxamide (4.25 g, 10.5 mmol, Intermediate G) in dioxane (30 mL) were added K₂CO₃ (3.63 g, 26.3 mmol) and RuPhos (408.89 mg, 0.876 mmol) in portions at rt under nitrogen atmosphere. To the above mixture was added RuPhos-PdCl-2nd G (681.48 mg, 0.876 mmol) in portions at rt. The resulting mixture was stirred for 16 h at 100° C. under nitrogen atmosphere. On completion, the reaction mixture was cooled to rt and was concentrated under reduced pressure. The precipitated solids were collected by filtration and washed with MeOH (3×50 mL). The crude solid was purified by reverse phase Flash chromatography (Column: WelFlash™ C18-I, 20-40 um, 330 g; Eluent A: Water (plus 10 mmol/L NH₄HCO₃); Eluent B: ACN; Gradient: 25%-55% B in 25 min; Flow rate: 80 mL/min; Detector: 254 nm; desired fractions were collected at 50% B) and concentrated under reduced pressure to afford the title compound (460 mg, 9% yield) as a brown solid. LC/MS (ESI, m/z): [(M+H)]⁺=596.2.

### Step 3—6-{3-[(3-{[(1R,2S)-2-fluorocyclopropyl]carbamoyl}-8-{[(4-methoxyphenyl)methyl](methyl)amino}imidazo[1,2-b]pyridazin-6-yl)amino]phenyl}pyridine-3-carboxylic Acid

To a stirred solution of methyl 6-{3-[(3-{[(1R,2S)-2-fluorocyclopropyl]carbamoyl}-8-{[(4-methoxyphenyl)methyl](methyl)amino}imidazo[1,2-b]pyridazin-6-yl)amino]phenyl}pyridine-3-carboxylate (460 mg, 0.77 mmol) in THF (10 mL) and H₂O (10 mL) was added LiOH (92.48 mg, 3.860 mmol) in portions at rt under nitrogen atmosphere. The resulting mixture was stirred for 3 h at rt under nitrogen atmosphere. On completion, the reaction mixture was concentrated under reduced pressure. The mixture was acidified to pH 5 with 2 M HCl. The resulting mixture was cooled to rt and diluted with water (100 mL), then extracted with DCM (3×100 mL). The combined organic layers were washed with brine (1×100 mL), and dried over anhydrous Na₂SO₄. After filtration, the filtrate was concentrated under reduced pressure to afford the title compound (360 mg, 80% yield) as a brown solid. LC/MS (ESI, m/z): [(M+H)]⁺=582.2.

### Step 4—6-{3-[(3-{[(1R,2S)-2-fluorocyclopropyl]carbamoyl}-8-(methylamino)imidazo[1,2-b]pyridazin-6-yl)amino]phenyl}pyridine-3-carboxylic Acid

To a stirred solution of 6-{3-[(3-{[(1R,2S)-2-fluorocyclopropyl]carbamoyl}-8-{[(4-methoxyphenyl)methyl](methyl)amino}imidazo[1,2-b]pyridazin-6-yl)amino]phenyl}pyridine-3-carboxylic acid (360 mg, 0.62 mmol) in DCM (5 mL) was added TFA (1 mL) dropwise at rt under nitrogen atmosphere. The resulting mixture was stirred for 1 h at rt under nitrogen atmosphere. On completion, the reaction mixture was concentrated under reduced pressure. The residue was purified by trituration with ethyl ether (50 mL) to afford the title compound (230 mg, 81% yield) as a brown solid. LC/MS (ESI, m/z): [(M+H)]⁺=462.3.

### Example 1.256. Preparation of 6-{[2-fluoro-3-(5-formylpyridin-2-yl)phenyl]amino}-N-[(1R,2S)-2-fluorocyclopropyl]-8-(methylamino)imidazo[1,2-b]pyridazine-3-carboxamide (Intermediate IY)

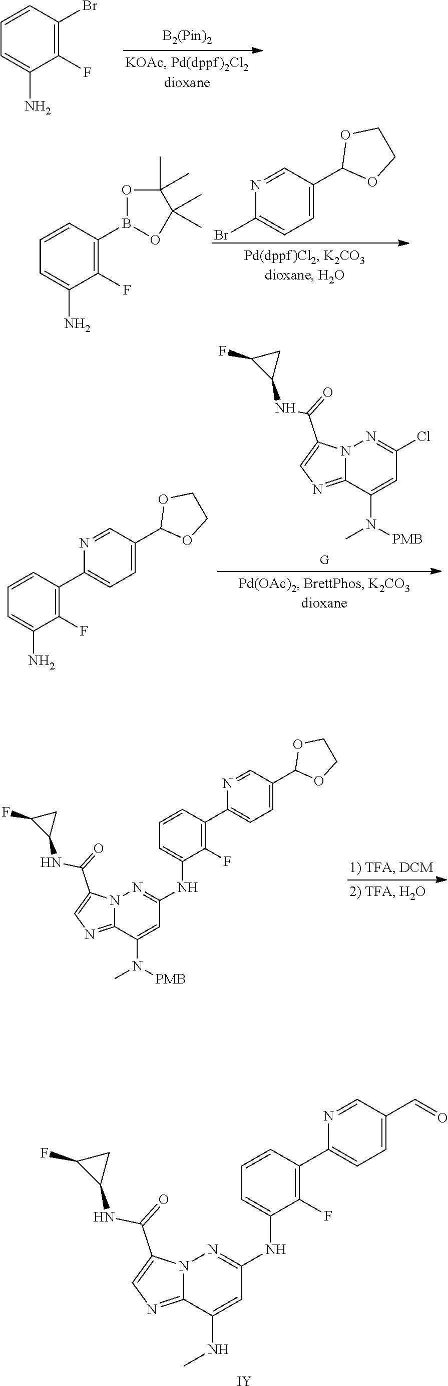

### Step 1—2-Fluoro-3-(4,4,5,5-tetramethyl-1,3,2-dioxaborolan-2-yl)aniline

To a stirred solution of 3-bromo-2-fluoroaniline (5.50 g, 29.0 mmol) and bis(pinacolato)diboron (14.70 g, 57.89 mmol) in dioxane (50 mL) was added KOAc (8.52 g, 86.8 mmol) and Pd(dppf)Cl₂ (2.12 g, 2.90 mmol) at rt under nitrogen atmosphere. The resulting mixture was stirred for 16 h at 90° C. under nitrogen atmosphere. On completion, the reaction was cooled to rt and concentrated under reduced pressure. The residue was purified by silica gel column chromatography, eluted with PE/EA (1:1), to afford the title compound (6.421 g, 94% yield) as a yellow solid. LC/MS (ESI, m/z): [(M+H)]⁺=238.2.

### Step 2—3-[5-(1,3-Dioxolan-2-yl)pyridin-2-yl]-2-fluoroaniline

To a stirred solution of 2-fluoro-3-(4,4,5,5-tetramethyl-1,3,2-dioxaborolan-2-yl)aniline (7.42 g, 31.3 mmol) and 2-bromo-5-(1,3-dioxolan-2-yl)pyridine (6.00 g, 26.1 mmol) in dioxane (50 mL) and H₂O (10 mL) were added K₂CO₃ (10.81 g, 78.24 mmol) and Pd(dppf)Cl₂ CH₂Cl₂ (2.12 g, 2.61 mmol) at rt under nitrogen atmosphere. The resulting mixture was stirred for 2 h at 90° C. under nitrogen atmosphere. On completion, the reaction was cooled to rt and concentrated under reduced pressure. The residue was purified by silica gel column chromatography, eluted with PE/EA (1:1), to afford the title compound (5.168 g, 76% yield) as a yellow solid. LC/MS (ESI, m/z): [(M+H)]⁺=261.1.

### Step 3—6-({3-[5-(1,3-dioxolan-2-yl)pyridin-2-yl]-2-fluorophenyl}amino)-N-[(1R,2S)-2-fluorocyclopropyl]-8-{[(4-methoxyphenyl)methyl](methyl)amino}imidazo[1,2-b]pyridazine-3-carboxamide

To a stirred solution of 3-[5-(1,3-dioxolan-2-yl)pyridin-2-yl]-2-fluoroaniline (650 mg, 2.50 mmol) and 6-chloro-N-[(1R,2S)-2-fluorocyclopropyl]-8-{[(4-methoxyphenyl)methyl](methyl)amino}imidazo[1,2-b]pyridazine-3-carboxamide (1009 mg, 2.499 mmol, Intermediate G) in dioxane (6 mL) was added K₂CO₃ (1035 mg, 7.489 mmol), BrettPhos (268 mg, 0.499 mmol) and Pd(OAc)₂ (56 mg, 0.249 mmol) at rt under nitrogen atmosphere. The resulting mixture was stirred for 4 h at 100° C. under nitrogen atmosphere. On completion, the reaction mixture was cooled to rt and concentrated under reduced pressure. The residue was purified by silica gel column chromatography, eluted with EA, to afford the title compound (0.93 g, 59% yield) as a yellow solid. LC/MS (ESI, m/z): [(M+H)]⁺=628.3.

### Step 4—6-{[2-Fluoro-3-(5-formylpyridin-2-yl)phenyl]amino}-N-[(1R,2S)-2-fluorocyclopropyl]-8-(methylamino)imidazo[1,2-b]pyridazine-3-carboxamide

A solution of 6-({3-[5-(1,3-dioxolan-2-yl)pyridin-2-yl]-2-fluorophenyl}amino)-N-[(1R,2S)-2-fluorocyclopropyl]-8-{[(4-methoxyphenyl)methyl](methyl)amino}imidazo[1,2-b]pyridazine-3-carboxamide (810 mg, 1.29 mmol) in DCM (7.5 mL) and TFA (2.5 mL) was stirred for 1 h at rt under nitrogen atmosphere. On completion, the resulting mixture was concentrated under reduced pressure. The residue was dissolved in TFA (5 mL) and H₂O (5 mL). The resulting mixture was then stirred for 2 h at 50° C. under nitrogen atmosphere. On completion, the reaction mixture was concentrated under reduced pressure. The residue was purified by trituration with Et₂O (15 mL) to afford the title compound (650 mg) as a white solid. LC/MS (ESI, m/z): [(M+H)]⁺=464.2.

### Example 1.257. Preparation of N-[(1R,2S)-2-fluorocyclopropyl]-6-[4-(4-formylpyridin-2-yl)-2,3-dihydroindol-1-yl]-8-(methylamino)imidazo[1,2-b]pyridazine-3-carboxamide (Intermediate IZ)

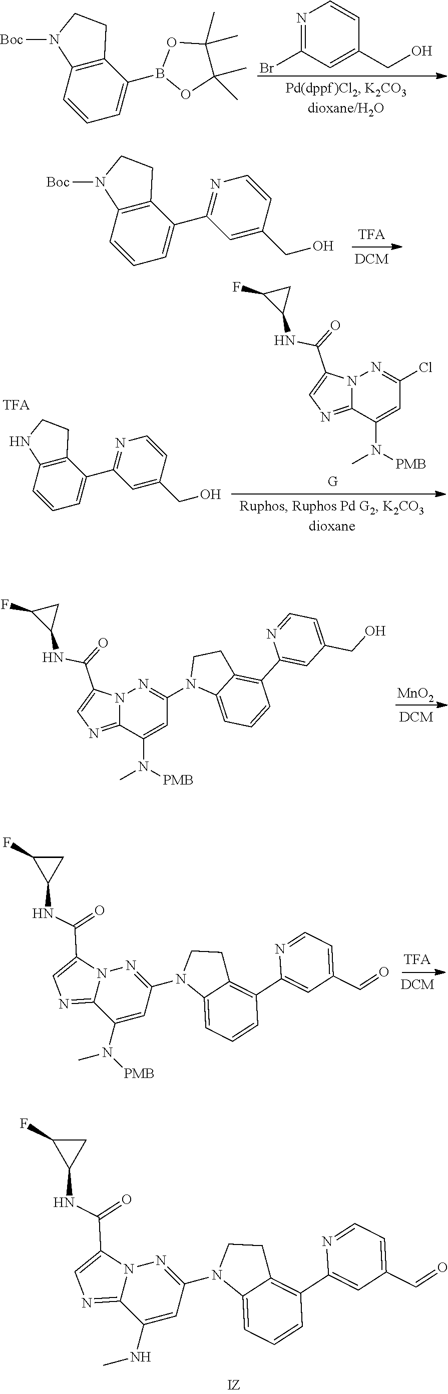

### Step 1—Tert-butyl 4-[4-(hydroxymethyl)pyridin-2-yl]-2,3-dihydroindole-1-carboxylate

To a stirred solution of tert-butyl 4-(4,4,5,5-tetramethyl-1,3,2-dioxaborolan-2-yl)-2,3-dihydroindole-1-carboxylate (5 g, 15 mmol) and (2-bromopyridin-4-yl)methanol (2.72 g, 14.5 mmol) in dioxane (20 mL) and H₂O (5 mL) was added K₂CO₃ (6.00 g, 43.5 mmol) at rt under nitrogen atmosphere. To the above mixture was added Pd(dppf)Cl₂CH₂Cl₂ (1.18 g, 1.45 mmol) at rt under nitrogen atmosphere. The resulting mixture was stirred for additional 5 min at rt, then the mixture was stirred for 2 h at 80° C. under nitrogen atmosphere. On completion, the mixture was cooled to rt and concentrated under reduced pressure. The residue was purified by silica gel column chromatography, eluted with PE/EA (6:1), to afford the title compound (2.5 g, 53% yield) as a light brown solid. LC/MS (ESI, m/z): [(M+H)]⁺=327.1.

### Step 2—2-(2,3-Dihydro-1H-indol-4-yl)pyridine-4-carbaldehyde Trifluoroacetate

To a stirred solution of tert-butyl 4-[4-(hydroxymethyl)pyridin-2-yl]-2,3-dihydroindole-1-carboxylate (2.5 g, 7.7 mmol) in DCM (20 mL) was added TFA (8 mL) at 0° C. under nitrogen atmosphere. Then the mixture was stirred for 2 h at rt under nitrogen atmosphere. On completion, the reaction mixture was concentrated under reduced pressure. The residue was purified by trituration with Et₂O (100 mL) to afford the title compound (1.2 g, 49% yield) as an off-white solid. LC/MS (ESI, m/z): [(M+H)]⁺=227.1.

### Step 3—N-[(1R,2S)-2-fluorocyclopropyl]-6-{4-[4-(hydroxymethyl)pyridin-2-yl]-2,3-dihydroindol-1-yl}-8-{[(4-methoxyphenyl)methyl](methyl)amino}imidazo[1,2-b]pyridazine-3-carboxamide

To a stirred solution of [2-(2,3-dihydro-1H-indol-4-yl)pyridin-4-yl]methanol trifluoroacetate (1 g, 3 mmol) and 6-chloro-N-[(1R,2S)-2-fluorocyclopropyl]-8-{[(4-methoxyphenyl)methyl](methyl)amino}imidazo[1,2-b]pyridazine-3-carboxamide (1.25 g, 3.08 mmol, Intermediate G) in dioxane (15 mL) were added RuPhos (0.29 g, 0.62 mmol) and K₂CO₃ (1.28 g, 9.25 mmol) at rt under argon atmosphere. The resulting mixture was stirred for additional 5 min at rt. Then, RuPhos-PdCl-2nd G (0.24 g, 0.308 mmol) was added in portions over 5 min at rt. The resulting mixture was stirred for additional 30 min at rt then at 100° C. for 16 h under nitrogen atmosphere. On completion, the mixture was cooled to rt and was concentrated under reduced pressure. The residue was purified by silica gel column chromatography, eluted with PE/EA (1:2), to afford the title compound (1 g, 55% yield) as a light brown solid. LC/MS (ESI, m/z): [(M+H)]⁺=594.2.

### Step 4—N-[(1R,2S)-2-fluorocyclopropyl]-6-[4-(4-formylpyridin-2-yl)-2,3-dihydroindol-1-yl]-8-{[(4-methoxyphenyl)methyl](methyl)amino}imidazo[1,2-b]pyridazine-3-carboxamide

To a stirred solution of N-[(1R,2S)-2-fluorocyclopropyl]-6-{4-[4-(hydroxymethyl)pyridin-2-yl]-2,3-dihydroindol-1-yl}-8-{[(4-methoxyphenyl)methyl](methyl)amino}imidazo[1,2-b]pyridazine-3-carboxamide (1.0 g, 1.7 mmol) in DCM (10 mL) was added MnO₂ (0.73 g, 8.4 mmol) at rt under nitrogen atmosphere. The resulting mixture was stirred for 16 h at 50° C. under nitrogen atmosphere. On completion, the resulting mixture was filtered and the filter cake was washed with DCM (60 mL). The filtrate was concentrated under reduced pressure. The residue was purified by silica gel column chromatography, eluted with PE/EA (1:1), to afford the title compound (560 mg, 56% yield) as an off-white solid. LC/MS (ESI, m/z): [(M+H)]⁺=592.2.

### Step 5—N-[(1R,2S)-2-fluorocyclopropyl]-6-[4-(4-formylpyridin-2-yl)-2,3-dihydroindol-1-yl]-8-(methylamino)imidazo[1,2-b]pyridazine-3-carboxamide

To a stirred solution of N-[(1R,2S)-2-fluorocyclopropyl]-6-[4-(4-formylpyridin-2-yl)-2,3-dihydroindol-1-yl]-8-{[(4-methoxyphenyl)methyl](methyl)amino}imidazo[1,2-b]pyridazine-3-carboxamide (100 mg, 0.2 mmol) in DCM (2 mL) was added TFA (0.5 mL) dropwise at 0° C. under nitrogen atmosphere. The resulting mixture was stirred for 2 h at rt under nitrogen atmosphere. On completion, the reaction mixture was concentrated under reduced pressure. The crude product (20 mg) was purified by prep-HPLC (Column: XBridge Prep Phenyl OBD Column, 19*150 mm, 5 μm; Mobile Phase A: Water (5 mmol/L NH₄HCO₃), Mobile Phase B: ACN; Flow rate: 60 mL/min; Gradient: 36% B to 46% B in 10 min, 46% B; Wave Length: 254 nm; RT1 (min): 8) to afford the title compound (6.2 mg, 7% yield) as a light yellow solid. ¹H NMR (400 MHz, DMSO-d₆) δ 10.19 (s, 1H), 8.98 (d, J=4.9 Hz, 1H), 8.88 (d, J=4.7 Hz, 1H), 8.20 (s, 1H), 7.97 (s, 1H), 7.84-7.77 (m, 2H), 7.73-7.69 (m, 1H), 7.43-7.32 (m, 2H), 6.02 (s, 1H), 5.00-4.73 (m, 1H), 4.23-4.12 (m, 2H), 3.52-3.45 (m, 2H), 3.08-3.02 (m, 1H), 2.97 (d, J=4.9 Hz, 3H), 1.30-1.18 (m, 1H), 1.03-0.93 (m, 1H). LC/MS (ESI, m/z): [(M+H)]⁺=472.2.

### Example 1.258. Preparation of 6-{3-[(3-{[(1R,2S)-2-fluorocyclopropyl]carbamoyl}-8-(methylamino)imidazo[1,2-b]pyridazin-6-yl)amino]-2-methoxyphenyl}pyridine-3-carboxylic Acid (Intermediate JA)

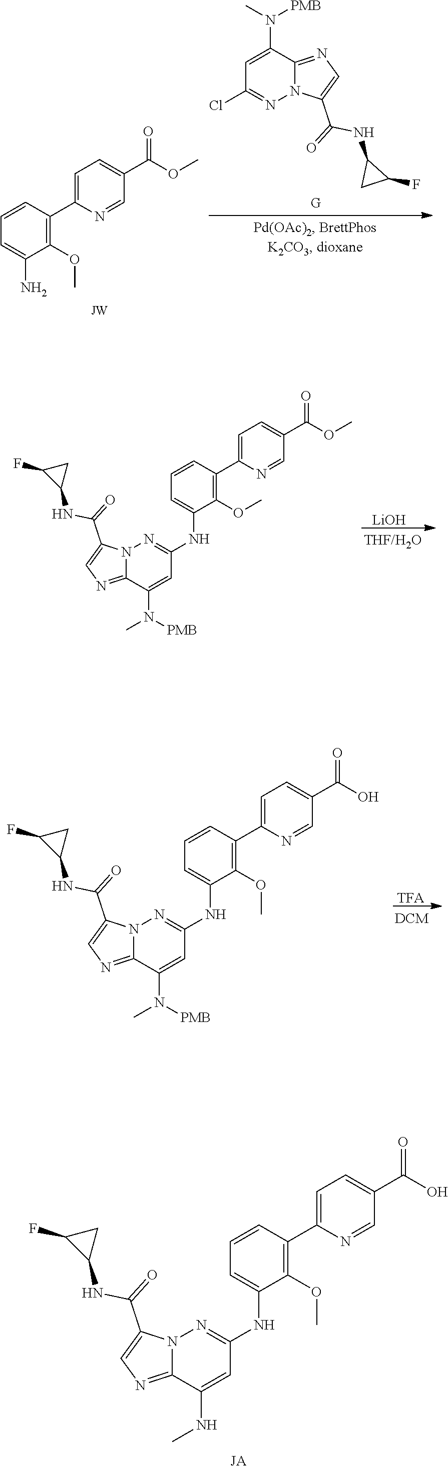

### Step 1—Methyl 6-{3-[(3-{[(1R,2S)-2-fluorocyclopropyl]carbamoyl}-8-{[(4-methoxyphenyl)methyl](methyl)amino}imidazo[1,2-b]pyridazin-6-yl)aminol-2-methoxyphenyl}pyridine-3-carboxylate

To a stirred solution of methyl 6-(3-amino-2-methoxyphenyl)pyridine-3-carboxylate (2.00 g, 7.74 mmol, Intermediate JW) and 6-chloro-N-[(1R,2S)-2-fluorocyclopropyl]-8-{[(4-methoxyphenyl)methyl](methyl)amino}imidazo[1,2-b]pyridazine-3-carboxamide (2.50 g, 6.20 mmol, Intermediate G) in dioxane (20 mL) was added K₂CO₃ (3.21 g, 23.2 mmol), BrettPhos (0.83 g, 1.6 mmol) and Pd(OAc)₂ (0.17 g, 0.77 mmol) at rt under nitrogen atmosphere. The resulting mixture was stirred for 4 h at 100° C. under nitrogen atmosphere. On completion, the reaction mixture was cooled to rt and was concentrated under reduced pressure. The residue was purified by silica gel column chromatography, eluted with EA, to afford the title compound (2.7 g, 56% yield) as a yellow solid. LC/MS (ESI, m/z): [(M+H)]⁺=626.3.

### Step 2—6-{3-[(3-{[(1R,2S)-2-fluorocyclopropyl]carbamoyl}-8-{[(4-methoxyphenyl)methyl](methyl)amino}imidazo[1,2-b]pyridazin-6-yl)amino]-2-methoxyphenyl}pyridine-3-carboxylic Acid

To a solution of methyl 6-{3-[(3-{[(1R,2S)-2-fluorocyclopropyl]carbamoyl}-8-{[(4-methoxyphenyl)methyl](methyl)amino}imidazo[1,2-b]pyridazin-6-yl)amino]-2-methoxyphenyl}pyridine-3-carboxylate (2.60 g, 4.16 mmol) in THF (15 mL) and H₂O (15 mL) was added LiOH (0.50 g, 20 mmol) at rt. The resulting mixture was stirred for 2 h at rt under nitrogen atmosphere. On completion, the THF was removed by concentration under reduced pressure. The water phase was acidified to pH 3 with conc. HCl. The precipitated solids were collected by filtration, washed with DCM (3×10 mL), and dried under vacuum to afford the title compound (2.1 g, 83% yield) as a white solid. LC/MS (ESI, m/z): [(M+H)]⁺=612.3.

### Step 3—6-{3-[(3-{[(1R,2S)-2-fluorocyclopropyl]carbamoyl}-8-(methylamino)imidazo[1,2-b]pyridazin-6-yl)amino]-2-methoxyphenyl}pyridine-3-carboxylic Acid

A solution of 6-{3-[(3-{[(1R,2S)-2-fluorocyclopropyl]carbamoyl}-8-{[(4-methoxyphenyl)methyl](methyl)amino}imidazo[1,2-b]pyridazin-6-yl)amino]-2-methoxyphenyl}pyridine-3-carboxylic acid (2 g, 3 mmol) in TFA (5 mL) and DCM (15 mL) was stirred for 1 h at rt under nitrogen atmosphere. On completion, the mixture was concentrated under reduced pressure. The residue was purified by trituration with Et₂O (10 mL) to afford the title compound (2.2 g, quant. yield) as a white solid. LC/MS (ESI, m/z): [(M+H)]⁺=492.3.

### Example 1.259. N-[(1R,2S)-2-fluorocyclopropyl]-6-({2-methoxy-3-[5-(piperazine-1-carbonyl)pyridin-2-yl]phenyl}amino)-8-(methylamino)imidazo[1,2-b]pyridazine-3-carboxamide (Intermediate JB)

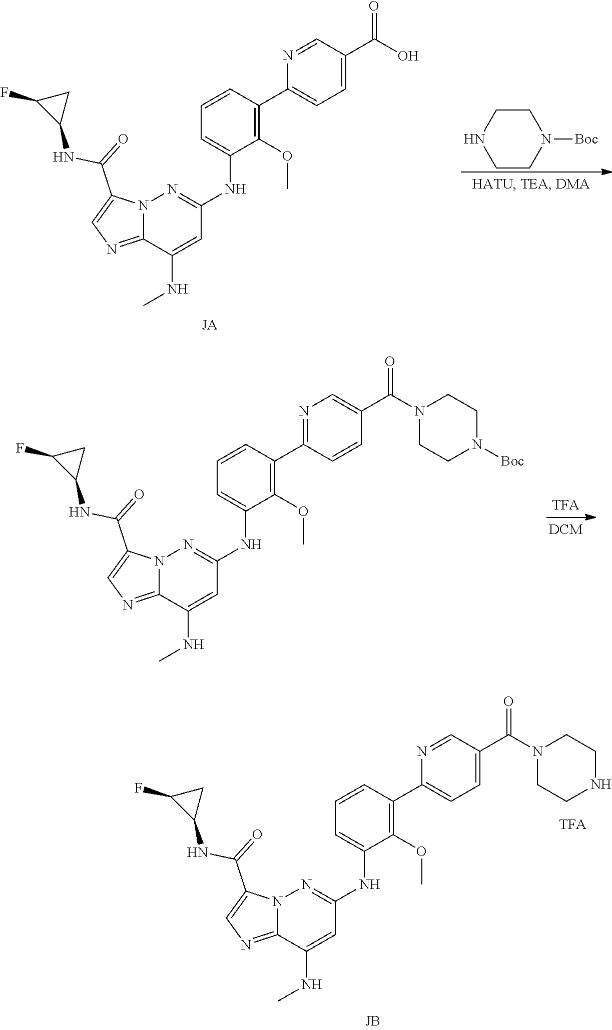 [SMILES: CNc1cc(Nc2cccc(-c3ccc(C(=O)O)cn3)c2OC)nn2c(C(=O)N[C@@H]3C[C@@H]3F)cnc12 ; CC(C)(C)OC(=O)N1CCNCC1 ; CNc1cc(Nc2cccc(-c3ccc(C(=O)N4CCN(C(=O)OC(C)(C)C)CC4)cn3)c2OC)nn2c(C(=O)N[C@@H]3C[C@@H]3F)cnc12 ; CNc1cc(Nc2cccc(-c3ccc(C(=O)N4CCNCC4)cn3)c2OC)nn2c(C(=O)N[C@@H]3C[C@@H]3F)cnc12]

### Step 1—Tert-butyl 4-(6-{3-[(3-{[(1R,2S)-2-fluorocyclopropyl]carbamoyl}-8-(methylamino)imidazo[1,2-b]pyridazin-6-yl)amino]-2-methoxyphenyl}pyridine-3-carbonyl)piperazine-1-carboxylate

To a stirred solution of 6-{3-[(3-{[(1R,2S)-2-fluorocyclopropyl]carbamoyl}-8-(methylamino)imidazo[1,2-b]pyridazin-6-yl)amino]-2-methoxyphenyl}pyridine-3-carboxylic acid (300 mg, 0.610 mmol, Intermediate JA) and tert-butyl piperazine-1-carboxylate (113.69 mg, 0.610 mmol) in DMA (7 mL) were added TEA (0.25 ml, 1.830 mmol) and HATU (348.14 mg, 0.915 mmol). The resulting mixture was stirred for 1 h at rt under nitrogen atmosphere. On completion, the reaction mixture was concentrated under vacuum. The residue was purified by reverse flash chromatography (Column: WelFlash™ C18-I, 20-40 um, 330 g; Eluent A: Water (plus 10 mmol/L FA); Eluent B: ACN; Gradient: 30%-50% B in 30 min; Flow rate: 80 mL/min; Detector: 220/254 nm; desired fractions were collected at 36% B) and concentrated under reduced pressure to afford the title compound (300 mg, 75% yield) as a white solid. LC/MS (ESI, m/z): [(M+H)]⁺=660.3.

### Step 2—N-[(1R,2S)-2-fluorocyclopropyl]-6-({2-methoxy-3-[5-(piperazine-1-carbonyl)pyridin-2-yl]phenyl}amino)-8-(methylamino)imidazo[1,2-b]pyridazine-3-carboxamide

To a solution of tert-butyl 4-(6-{3-[(3-{[(1R,2S)-2-fluorocyclopropyl]carbamoyl}-8-(methylamino)imidazo[1,2-b]pyridazin-6-yl)amino]-2-methoxyphenyl}pyridine-3-carbonyl)piperazine-1-carboxylate (300 mg, 0.5 mmol) in DCM (3 mL) was added TFA (1.5 mL) dropwise at rt. The reaction mixture was stirred for 1 h at rt under nitrogen atmosphere. On completion, the reaction mixture was concentrated under reduced pressure. The residue was triturated with Et₂O to afford the title compound (300 mg) as a yellow solid. LC/MS (ESI, m/z): [(M+H)]⁺=560.3.

### Example 1.260. 3-{5-[4-(3,3-difluoropiperidin-4-yl)piperazin-1-yl]-3-methyl-2-oxo-1,3-benzodiazol-1-yl}piperidine-2,6-dione (Intermediate JC)

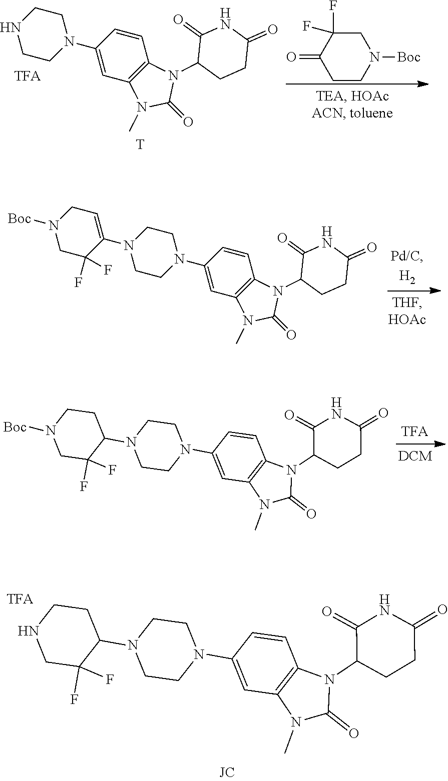

### Step 1—Tert-butyl 4-{4-[1-(2,6-dioxopiperidin-3-yl)-3-methyl-2-oxo-1,3-benzodiazol-5-yl]piperazin-1-yl}-3,3-difluoro-2,6-dihydropyridine-1-carboxylate

To a stirred solution of 3-[3-methyl-2-oxo-5-(piperazin-1-yl)-1,3-benzodiazol-1-yl]piperidine-2,6-dione trifluoroacetate (2.7 g, 6.1 mmol, Intermediate T) in toluene (20 mL) and ACN (20 mL) was added TEA (1.28 mL, 9.18 mmol) dropwise at rt under nitrogen atmosphere. To the above mixture was added tert-butyl 3,3-difluoro-4-oxopiperidine-1-carboxylate (5.76 g, 24.5 mmol) and AcOH (1.10 g, 18.4 mmol) at rt. The resulting mixture was stirred for additional 30 min at rt then at 110° C. for 16 hr under nitrogen atmosphere. On completion, the mixture was cooled to rt and was basified to pH 8 with saturated NaHCO₃ (aq.). The resulting mixture was extracted with EtOAc (3×100 mL). The combined organic layers were washed with brine (300 mL), and dried over anhydrous Na₂SO₄. After filtration, the filtrate was concentrated under reduced pressure to afford the title compound (3 g, 88% yield) as a colorless oil. LC/MS (ESI, m/z): [(M+H)]⁺=561.4.

### Step 2—Tert-butyl 4-{4-[1-(2,6-dioxopiperidin-3-yl)-3-methyl-2-oxo-1,3-benzodiazol-5-yl]piperazin-1-yl}-3,3-difluoropiperidine-1-carboxylate

To a solution of tert-butyl 4-{4-[1-(2,6-dioxopiperidin-3-yl)-3-methyl-2-oxo-1,3-benzodiazol-5-yl]piperazin-1-yl}-3,3-difluoro-2,6-dihydropyridine-1-carboxylate (3 g, 5 mmol) in THF (30 mL) was added Pd/C (4.29 g, 74.8 mmol) under nitrogen atmosphere. The reaction system was degassed under vacuum and purged with H₂ several times, then the mixture was hydrogenated under H₂ balloon (˜1 atm) at 25° C. for 3 h. On completion, Pd/C was filtered off through celite and the filter cake was washed with THF (3×30 mL). The residue was purified by silica gel column chromatography, eluted with CHCl₃/MeOH (10:1), to afford the title compound (850 mg, 28% yield) as a white solid. LC/MS (ESI, m/z): [(M+H)]⁺=563.3.

### Step 3—3-{5-[4-(3,3-Difluoropiperidin-4-yl)piperazin-1-yl]-3-methyl-2-oxo-1,3-benzodiazol-1-yl}piperidine-2,6-dione Trifluoroacetate

To a stirred solution of tert-butyl 4-{4-[1-(2,6-dioxopiperidin-3-yl)-3-methyl-2-oxo-1,3-benzodiazol-5-yl]piperazin-1-yl}-3,3-difluoropiperidine-1-carboxylate (850 mg, 1.51 mmol) in DCM (10 mL) was added TFA (4 mL) at 0° C. under nitrogen atmosphere. The resulting mixture was stirred for 2 h at rt under nitrogen atmosphere. On completion, the mixture was concentrated under reduced pressure. The residue was purified by trituration with Et₂O (60 mL) to afford the title compound (800 mg, 95% yield) as an off-white solid. LC/MS (ESI, m/z): [(M+H)]⁺=463.2.

### Example 1.261. 6-{[5-fluoro-3-(5-formylpyridin-2-yl)-2-methoxyphenyl]amino}-N-[(1R,2S)-2-fluorocyclopropyl]-8-(methylamino)imidazo[1,2-b]pyridazine-3-carboxamide (Intermediate JD)

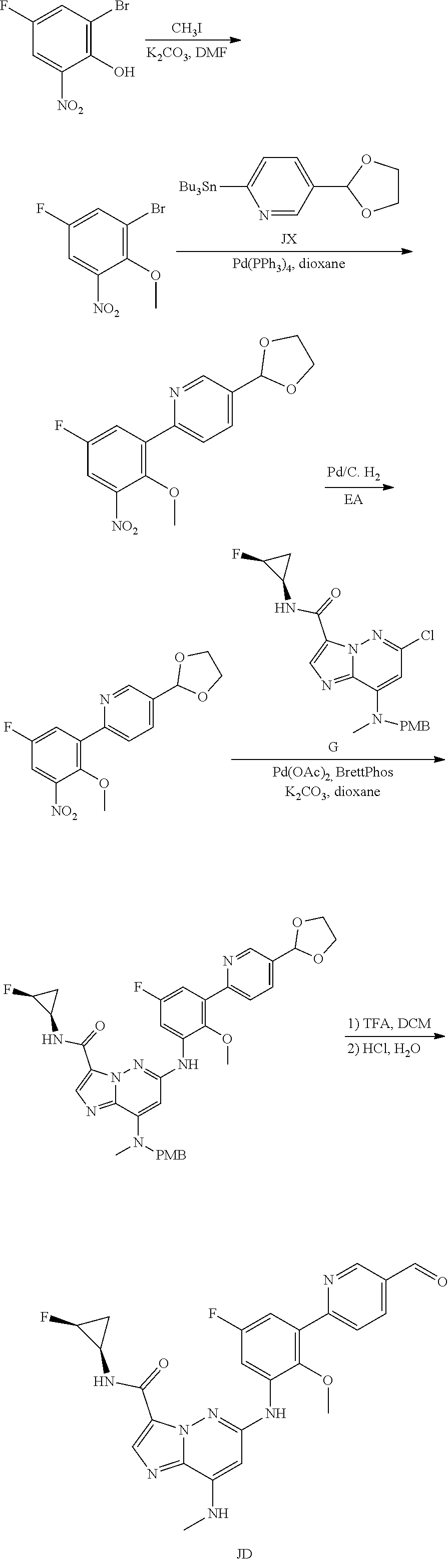

### Step 1—1-Bromo-5-fluoro-2-methoxy-3-nitrobenzene

To a stirred mixture of 3-bromo-5-fluoro-2-hydroxycyclohexylazinic acid (1 g, 4 mmol) and K₂CO₃ (1.70 g, 12.3 mmol) in DMF (20 mL) was added methyl iodide (1.74 g, 12.3 mmol) dropwise at rt and the resulting mixture was stirred for 16 h at rt. On completion, the mixture was diluted with water (50 mL) and extracted with EtOAc (3×50 mL). The combined organic layers were washed with brine (3×100 mL), and dried over anhydrous Na₂SO₄. After filtration, the filtrate was concentrated under reduced pressure. The residue was purified by silica gel column chromatography, eluted with PE/EA (9:1) to afford the title compound (0.95 g, 93% yield) as a light yellow solid. LC/MS (ESI, m/z): [(M+H)]⁺=250.1, 252.1.

### Step 2—5-(1,3-Dioxolan-2-yl)-2-(5-fluoro-2-methoxy-3-nitrophenyl)pyridine

To a stirred solution of 1-bromo-5-fluoro-2-methoxy-3-nitrobenzene (2 g, 8 mmol) and 5-(1,3-dioxolan-2-yl)-2-(tributylstannyl)pyridine (5282.09 mg, 11.998 mmol, Intermediate JX) in dioxane (20 mL) was added Pd(PPh₃)₄ (924.39 mg, 0.800 mmol) at rt under nitrogen atmosphere. Then the mixture was stirred for 16 h at 80° C. under nitrogen atmosphere. On completion, the reaction mixture was cooled to rt and concentrated under reduced pressure. The residue was purified by silica gel column chromatography, eluted with PE/EA (3:1), to afford the title compound (600 mg, 23% yield) as a brown solid. LC/MS (ESI, m/z): [(M+H)]⁺=321.1.

### Step 3—3-[5-(1,3-Dioxolan-2-yl)pyridin-2-yl]-5-fluoro-2-methoxyaniline

To a stirred solution of 5-(1,3-dioxolan-2-yl)-2-(5-fluoro-2-methoxy-3-nitrophenyl)pyridine (500 mg, 2 mmol) in EtOAc (10 mL) was added Pd/C (10 w %, 300 mg) under nitrogen atmosphere. The mixture was hydrogenated at rt for 2 h under hydrogen atmosphere using a hydrogen balloon. After completion, the reaction mixture was filtered through a Celite pad. The filter cake was washed with DCM:MeOH (1:1) (3×10 mL). The filtrate was concentrated under reduced pressure to afford the title compound (400 mg, 88% yield) as a brown solid. LC/MS (ESI, m/z): [(M+H)]⁺=291.2.

### Step 4—6-({3-[5-(1,3-Dioxolan-2-yl)pyridin-2-yl]-5-fluoro-2-methoxyphenyl}amino)-N-[(1R,2S)-2-fluorocyclopropyl]-8-{[(4-methoxyphenyl)methyl](methyl)amino}imidazo[1,2-b]pyridazine-3-carboxamide

To a stirred mixture of 3-[5-(1,3-dioxolan-2-yl)pyridin-2-yl]-5-fluoro-2-methoxyaniline (400 mg, 1 mmol) and 6-chloro-N-[(1R,2S)-2-fluorocyclopropyl]-8-{[(4-methoxyphenyl)methyl](methyl)amino}imidazo[1,2-b]pyridazine-3-carboxamide (556.46 mg, 1.378 mmol, Intermediate G) in dioxane (10 mL) was added BrettPhos (73.96 mg, 0.138 mmol), Pd(OAc)₂ (30.94 mg, 0.138 mmol) and K₃PO₄ (877.45 mg, 4.134 mmol) at rt under nitrogen atmosphere. The resulting mixture was stirred for 1 h at 100° C. under nitrogen atmosphere. On completion, the reaction mixture was cooled to rt and concentrated under reduced pressure The residue was purified by reverse flash chromatography (column, C18 silica gel; mobile phase, MeCN in Water (10 mmol/L NH₄HCO₃), 30% to 80% gradient in 30 min; detector, UV 254 nm) to afford the title compound (450 mg, 50% yield) as a light yellow solid. LC/MS (ESI, m/z): [(M+H)]⁺=658.3.

### Step 5—6-{[5-Fluoro-3-(5-formylpyridin-2-yl)-2-methoxyphenyl]amino}-N-[(1R,2S)-2-fluorocyclopropyl]-8-(methylamino)imidazo[1,2-b]pyridazine-3-carboxamide

A solution of 6-({3-[5-(1,3-dioxolan-2-yl)pyridin-2-yl]-5-fluoro-2-methoxyphenyl}amino)-N-[(1R,2S)-2-fluorocyclopropyl]-8-{[(4-methoxyphenyl)methyl](methyl)amino}imidazo[1,2-b]pyridazine-3-carboxamide (350 mg, 0.53 mmol) in DCM (10 ml) was added TFA (2 ml) and the was stirred for 1 h at rt. On completion, the mixture was concentrated under reduced pressure. The residue was then dissolved in 2 M HCl (2 mL), and the mixture was stirred for 1 h at 50° C. On completion, the reaction mixture was cooled to rt and concentrated under reduced pressure. The residue was purified by reverse flash chromatography (column, C18 silica gel; mobile phase, MeCN in Water (10 mmol/L NH₄HCO₃), 5% to 70% gradient in 30 min; detector, UV 254 nm) to afford the title compound (170 mg) as an off-white solid. LC/MS (ESI, m/z): [(M+H)]⁺=494.2.

### Example 1.262. Preparation of N-[(1R,2S)-2-fluorocyclopropyl]-8-{[3-(5-formylpyridin-2-yl)phenyl]amino}-10-oxa-3,6,7,13-tetraazatricyclo[7.4.0.0\hat{ }{2,6}]trideca-1(9),2,4,7-tetraene-5-carboxamide (Intermediate JE)

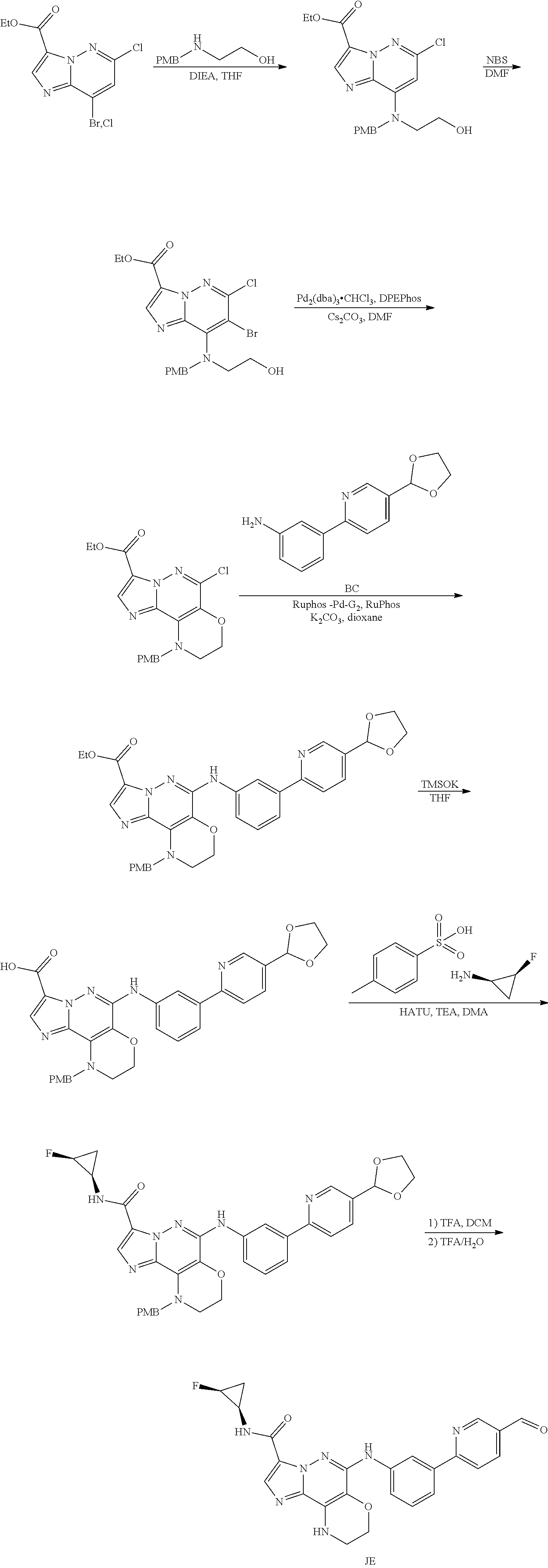

### Step 1—Ethyl 6-chloro-8-[(2-hydroxyethyl)[(4-methoxyphenyl)methyl]amino]imidazo[1,2-b]pyridazine-3-carboxylate

To a stirred solution of ethyl 6,8-dichloroimidazo[1,2-b]pyridazine-3-carboxylate (10.00 g, 38.45 mmol, synthesized via Step 1 of Intermediate F) and 2-{[(4-methoxyphenyl)methyl]amino}ethanol (9.06 g, 50.0 mmol) in THF (100 mL) was added DIEA (14.91 g, 115.4 mmol) at rt under nitrogen atmosphere. The resulting mixture was stirred for 16 h at rt under nitrogen atmosphere. On completion, the reaction mixture was concentrated under reduced pressure. The residue was purified by silica gel column chromatography, eluted with PE/EA (1:1) to afford the title compound (8.366 g, 54% yield) as a yellow oil. LC/MS (ESI, m/z): [(M+1)]⁺=405.1.

### Step 2—Ethyl 7-bromo-6-chloro-8-[(2-hydroxyethyl)[(4-methoxyphenyl)methyl]amino]imidazo[1,2-b]pyridazine-3-carboxylate

To a stirred solution of ethyl 6-chloro-8-[(2-hydroxyethyl)[(4-methoxyphenyl)methyl]amino]imidazo[1,2-b]pyridazine-3-carboxylate (8.20 g, 20.3 mmol) in DMF (80 mL) was added NBS (3.97 g, 22.3 mmol) at rt under nitrogen atmosphere and the mixture was stirred for 1 h at rt. On completion, the reaction mixture was extracted with EtOAc (3×100 mL). The combined organic layers were washed with brine (1×100 mL), and dried over anhydrous Na₂SO₄. After filtration, the filtrate was concentrated under reduced pressure. The residue was purified by silica gel column chromatography, eluted with PE/EA (1:1), to afford the title compound (7.60 g, 78% yield) as a yellow solid. LC/MS (ESI, m/z): [(M+1)]⁺=482.9.

### Step 3—Ethyl 8-chloro-13-[(4-methoxyphenyl)methyl]-10-oxa-3,6,7,13-tetraazatricyclo[7.4.0.0\hat{ }{2,6}]trideca-1(9),2,4,7-tetraene-5-carboxylate

To a stirred solution of ethyl 7-bromo-6-chloro-8-[(2-hydroxyethyl)[(4-methoxyphenyl)methyl]amino]imidazo[1,2-b]pyridazine-3-carboxylate (2.00 g, 4.13 mmol) and Cs₂CO₃ (2.69 g, 8.27 mmol) in DMF (20 mL) was added {2-[2-(diphenylphosphanyl)phenoxy]phenyl}diphenylphosphane (0.22 g, 0.41 mmol) and Pd₂(dba)₃·CHCl₃ (0.43 g, 0.41 mmol) at rt under nitrogen atmosphere. The resulting mixture was then stirred for 16 h at 100° C. under nitrogen atmosphere. On completion, the reaction mixture was cooled to rt and extracted with EtOAc (3×50 mL). The combined organic layers were washed with brine (1×50 mL), and dried over anhydrous Na₂SO₄. After filtration, the filtrate was concentrated under reduced pressure. The residue was purified by silica gel column chromatography, eluted with PE/EA (1:1), to afford the title compound (1.00 g, 60% yield) as a yellow oil.

### Step 4—Ethyl 8-({3-[5-(1,3-dioxolan-2-yl)pyridin-2-yl]phenyl}amino)-13-[(4-methoxyphenyl)methyl]-10-oxa-3,6,7,13-tetraazatricyclo[7.4.0.0\hat{ }{2,6}]trideca-1(9),2,4,7-tetraene-5-carboxylate

To a stirred solution of ethyl 8-chloro-13-[(4-methoxyphenyl)methyl]-10-oxa-3,6,7,13-tetraazatricyclo[7.4.0.0\hat{ }{2,6}]trideca-1(9),2,4,7-tetraene-5-carboxylate (600 mg, 2 mmol) and 3-[5-(1,3-dioxolan-2-yl)pyridin-2-yl]aniline (360.85 mg, 2 mmol, Intermediate BC) in dioxane (10 mL) were added K₂CO₃ (617.54 mg, 4.467 mmol), RuPhos (139.01 mg, 0.298 mmol) and RuPhos-PdCl-2nd G (115.84 mg, 0.149 mmol) at rt under nitrogen atmosphere. The resulting mixture was purged with nitrogen three times and stirred for 2 h at 100° C. under nitrogen atmosphere. On completion, the mixture was cooled to rt and concentrated under reduced pressure. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 40%-70% B in 25 min; Flow rate: 80 mL/min; Detector: 220/254 nm; desired fractions were collected at 65% B) and concentrated under reduced pressure to afford the title compound (550 mg, 61% yield) as a brown solid. LC/MS (ESI, m/z): [(M+1)]⁺=609.3.

### Step 5—8-({3-[5-(1,3-Dioxolan-2-yl)pyridin-2-yl]phenyl}amino)-13-[(4-methoxyphenyl)methyl]-10-oxa-3,6,7,13-tetraazatricyclo[7.4.0.0\hat{ }{2,6}]trideca-1(9),2,4,7-tetraene-5-carboxylic Acid

To a stirred solution of ethyl 8-({3-[5-(1,3-dioxolan-2-yl)pyridin-2-yl]phenyl}amino)-13-[(4-methoxyphenyl)methyl]-10-oxa-3,6,7,13-tetraazatricyclo[7.4.0.0\hat{ }{2,6}]trideca-1(9),2,4,7-tetraene-5-carboxylate (550 mg, 0.90 mmol) in THF (4 mL) was added potassium trimethylsilanolate (231.85 mg, 1.808 mmol) at rt under nitrogen atmosphere. The resulting mixture was stirred for 5 h at rt under nitrogen atmosphere. On completion, the mixture was acidified to pH 5 with FA and concentrated under vacuum. The residue was purified by reverse phase flash chromatography (Column: WelFlash™ C18-I, 20-40 μm, 330 g; Eluent A: Water (plus 10 mmol/L FA); Eluent B: ACN; Gradient: 35%-65% B in 25 min; Flow rate: 80 mL/min; Detector: 220/254 nm; desired fractions were collected at 45% B) and concentrated under reduced pressure to afford the title compound (320 mg, 61% yield) as a yellow solid. LC/MS (ESI, m/z): [(M+1)]⁺=581.2.

### Step 6—8-({3-[5-(1,3-Dioxolan-2-yl)pyridin-2-yl]phenyl}amino)-N-[(1R,2S)-2-fluorocyclopropyl]-13-[(4-methoxyphenyl)methyl]-10-oxa-3,6,7,13-tetraazatricyclo[7.4.0.0\hat{ }{2,6}]trideca-1(9),2,4,7-tetraene-5-carboxamide

To a stirred solution of 8-({3-[5-(1,3-dioxolan-2-yl)pyridin-2-yl]phenyl}amino)-13-[(4-methoxyphenyl)methyl]-10-oxa-3,6,7,13-tetraazatricyclo[7.4.0.0\hat{ }{2,6}]trideca-1(9),2,4,7-tetraene-5-carboxylic acid (320 mg, 0.55 mmol) and (1R,2S)-2-fluorocyclopropan-1-amine para-toluene sulfonate (136.29 mg, 0.551 mmol) in DMA (6 mL) were added HATU (314.35 mg, 0.827 mmol) and DIEA (0.3 mL) at rt under nitrogen atmosphere. The resulting mixture was stirred for 1 h at rt under nitrogen atmosphere. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 45%-70% B in 25 min; Flow rate: 80 mL/min; Detector: 220/254 nm; desired fractions were collected at 60% B) and concentrated under reduced pressure to afford the title compound (200 mg, 57% yield) as a yellow solid. LC/MS (ESI, m/z): [(M+1)]⁺=638.3.

### Step 7—N-[(1R,2S)-2-fluorocyclopropyl]-8-{[3-(5-formylpyridin-2-yl)phenyl]amino}-10-oxa-3,6,7,13-tetraazatricyclo[7.4.0.0\hat{ }{2,6}]trideca-1(9),2,4,7-tetraene-5-carboxamide

To a stirred solution of 8-({3-[5-(1,3-dioxolan-2-yl)pyridin-2-yl]phenyl}amino)-N-[(1R,2S)-2-fluorocyclopropyl]-13-[(4-methoxyphenyl)methyl]-10-oxa-3,6,7,13-tetraazatricyclo[7.4.0.0\hat{ }{2,6}]trideca-1(9),2,4,7-tetraene-5-carboxamide (200 mg, 0.3 mmol) in DCM (3 mL) was added TFA (2 mL) at rt under nitrogen atmosphere and the mixture was stirred for 1 h. On completion, the mixture was concentrated under reduced pressure. To the above residue were added H₂O (4 mL) and TFA (2 mL) at rt. The resulting mixture was then stirred for additional 4 h at 50° C. On completion, the reaction mixture was cooled to rt and basified to pH 8 with saturated Na₂CO₃ (aq.). The resulting mixture was extracted with EtOAc (3×50 mL). The combined organic layers were washed with brine (1×50 mL), and dried over anhydrous Na₂SO₄. After filtration, the filtrate was concentrated under reduced pressure. The residue was purified by reverse phase flash chromatography (Column: WelFlash™ C18-I, 20-40 μm, 120 g; Eluent A: Water (plus 10 mmol/L FA); Eluent B: ACN; Gradient: 25%-55% B in 25 min; Flow rate: 60 mL/min; Detector: 220/254 nm; desired fractions were collected at 34% B) and concentrated under reduced pressure to afford the title compound (104.4 mg, 70% yield) as a white solid. LC/MS (ESI, m/z): [(M+1)]⁺=474.2.

### Example 1.263. Preparation of N-[(1R,2S)-2-fluorocyclopropyl]-6-{[3-(4-formylpiperidin-1-yl)-2-methoxyphenyl]amino}-8-(methylamino)imidazo[1,2-b]pyridazine-3-carboxamide (Intermediate JF)

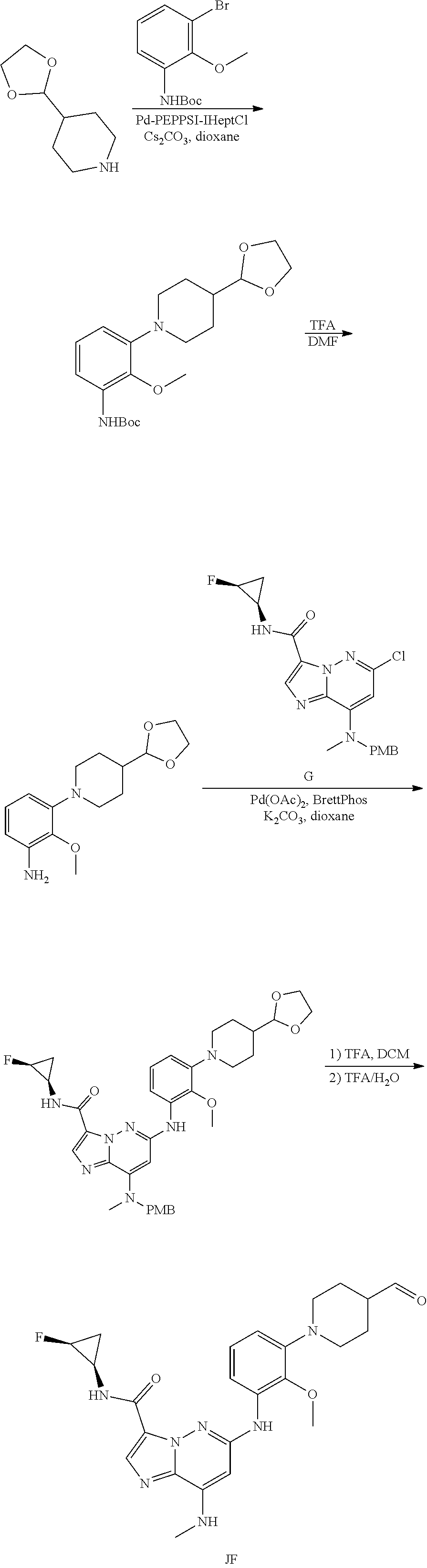

### Step 1—Tert-butyl N-{3-[4-(1,3-dioxolan-2-yl)piperidin-1-yl]-2-methoxyphenyl}carbamate

To a stirred mixture of 4-(1,3-dioxolan-2-yl)piperidine (2.08 g, 13.2 mmol, synthesized via Steps 1-2 of Intermediate HV) and tert-butyl N-(3-bromo-2-methoxyphenyl)carbamate (2 g, 7 mmol) in dioxane (50 mL) was added Cs₂CO₃ (6.47 g, 19.9 mmol) and Pd-PEPPSI-IHeptCl 3-chloropyridine (0.64 g, 0.66 mmol) in portions at rt under nitrogen atmosphere. The resulting mixture was stirred for 2 h at 100° C. under nitrogen atmosphere. On completion, the mixture was cooled to rt and concentrated under vacuum. The residue was purified by reversed-phase flash chromatography (column, C18 silica gel; mobile phase, ACN in Water (10 mmol/L NH₄HCO₃), 30% to 50% gradient in 25 min; detector, UV 254 nm) to afford the title compound (1.3 g, 52% yield) as a brown solid. LC/MS (ESI, m/z): [(M+H)]⁺=379.3.

### Step 2—3-[4-(1,3-Dioxolan-2-yl)piperidin-1-yl]-2-methoxyaniline

To a solution of tert-butyl N-{3-[4-(1,3-dioxolan-2-yl)piperidin-1-yl]-2-methoxyphenyl}carbamate (1.30 g, 3.44 mmol) in DCM (10 mL) was added TFA (2 mL) dropwise at rt. The resulting mixture was stirred for 1.5 h at rt under nitrogen atmosphere. On completion, the solution was basified to pH 10 with saturated aq. Na₂CO₃. The resulting mixture was extracted with EA (3×50 mL). The combined organic layers were washed with brine (5×30 mL), and dried over anhydrous Na₂SO₄. After filtration, the filtrate was concentrated under reduced pressure to give the title compound (900 mg, 94% yield) as a brown oil. LC/MS (ESI, m/z): [(M+H)]⁺=279.3.

### Step 3—6-({3-[4-(1,3-Dioxolan-2-yl)piperidin-1-yl]-2-methoxyphenyl}amino)-N-[(1R,2S)-2-fluorocyclopropyl]-8-{[(4-methoxyphenyl)methyl](methyl)amino}imidazo[1,2-b]pyridazine-3-carboxamide

To a stirred mixture of 3-[4-(1,3-dioxolan-2-yl)piperidin-1-yl]-2-methoxyaniline (880 mg, 3.16 mmol) and 6-chloro-N-[(1R,2S)-2-fluorocyclopropyl]-8-{[(4-methoxyphenyl)methyl](methyl)amino}imidazo[1,2-b]pyridazine-3-carboxamide (1.40 g, 3.48 mmol, Intermediate G) in dioxane (10 mL) were added RuPhos-PdOMs-2nd G (265 mg, 0.316 mmol), RuPhos (295 mg, 0.632 mmol) and K₂CO₃ (1.318 g, 9.484 mmol) in portions at rt under nitrogen atmosphere. The resulting mixture was stirred for 2 h at 100° C. under nitrogen atmosphere. On completion, the mixture was cooled to rt and concentrated under vacuum. The residue was purified by silica gel column chromatography, eluted with PE/EA (5:1˜1:10), to afford the title compound (1.30 g, 64% yield) as a brown solid. LC/MS (ESI, m/z): [(M+H)]⁺=646.4.

### Step 4—N-[(1R,2S)-2-fluorocyclopropyl]-6-{[3-(4-formylpiperidin-1-yl)-2-methoxyphenyl]amino}-8-(methylamino)imidazo[1,2-b]pyridazine-3-carboxamide

A mixture of 6-({3-[4-(1,3-dioxolan-2-yl)piperidin-1-yl]-2-methoxyphenyl}amino)-N-[(1R,2S)-2-fluorocyclopropyl]-8-{[(4-methoxyphenyl)methyl](methyl)amino}imidazo[1,2-b]pyridazine-3-carboxamide (1.30 g, 2.01 mmol) in TFA (10 mL) and DCM (10 mL) was stirred for 1.5 h at rt. After removal of the PMB group, the mixture was concentrated under vacuum. The residue was re-dissolved in TFA (10 mL) and water (10 mL) and stirred for 3 h at 50° C. On completion, the mixture was concentrated under vacuum. The residue was purified by trituration with diethyl ether (10 mL) to afford the title compound (1.30 g, quant. yield) as an off-white solid. LC/MS (ESI, m/z): [(M+H)]⁺=482.3.

### Example 1.264. Preparation of 2-Chloro-3-[5-(1,3-dioxolan-2-yl)pyridin-2-yl]aniline (Intermediate JG)

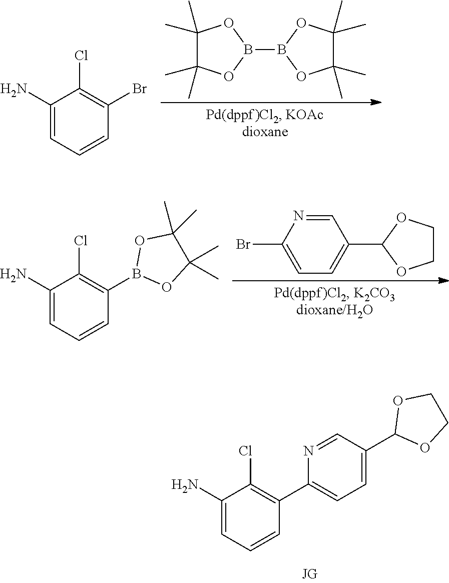

### Step 1—2-Chloro-3-(4,4,5,5-tetramethyl-1,3,2-dioxaborolan-2-yl)aniline

To a stirred mixture of 3-bromo-2-chloroaniline (20 g, 100 mmol) and bis(pinacolato)diboron (73.79 g, 290.6 mmol) in dioxane (200 mL) were added Pd(dppf)Cl₂·CH₂Cl₂ (3.95 g, 4.84 mmol) and KOAc (28.52 g, 290.6 mmol) at rt under nitrogen atmosphere. The resulting mixture was stirred for 4 h at 90° C. under nitrogen atmosphere. On completion, the mixture was cooled to rt and concentrated under vacuum. The residue was purified by silica gel column chromatography, eluted with PE/EA (10:1), to afford the title compound (23 g, 94% yield) as a light yellow solid. LC/MS (ESI, m/z): [(M+H)]⁺=254.2.

### Step 2—2-Chloro-3-[5-(1,3-dioxolan-2-yl)pyridin-2-yl]aniline

To a stirred mixture of 2-chloro-3-(4,4,5,5-tetramethyl-1,3,2-dioxaborolan-2-yl)aniline (6 g, 23.666 mmol) and 2-bromo-5-(1,3-dioxolan-2-yl)pyridine (6.53 g, 28.4 mmol, synthesized via Step 1 of Intermediate BC) in dioxane (60 mL) were added Pd(dppf)Cl₂·CH₂Cl₂ (1.93 g, 2.37 mmol) and K₂CO₃ (9.81 g, 71.0 mmol) at rt under nitrogen atmosphere. The resulting mixture was stirred for 4 h at 90° C. under nitrogen atmosphere. On completion, the mixture was cooled to rt and concentrated under reduced pressure. The residue was purified by silica gel column chromatography, eluted with PE/EA (5:1) to afford the title compound (5 g, 76% yield) as a light yellow solid. LC/MS (ESI, m/z): [(M+H)]⁺=277.2.

### Example 1.254. Preparation of 8-{[2-chloro-4′-(1,3-dioxolan-2-yl)-[1,1′-biphenyl]-3-yl]amino}-N-[(1R,2S)-2-fluorocyclopropyl]-10-oxa-3,6,7,13-tetraazatricyclo[7.4.0.0\hat{ }{2,6}]trideca-1(9),2,4,7-tetraene-5-carboxamide (Intermediate JH)

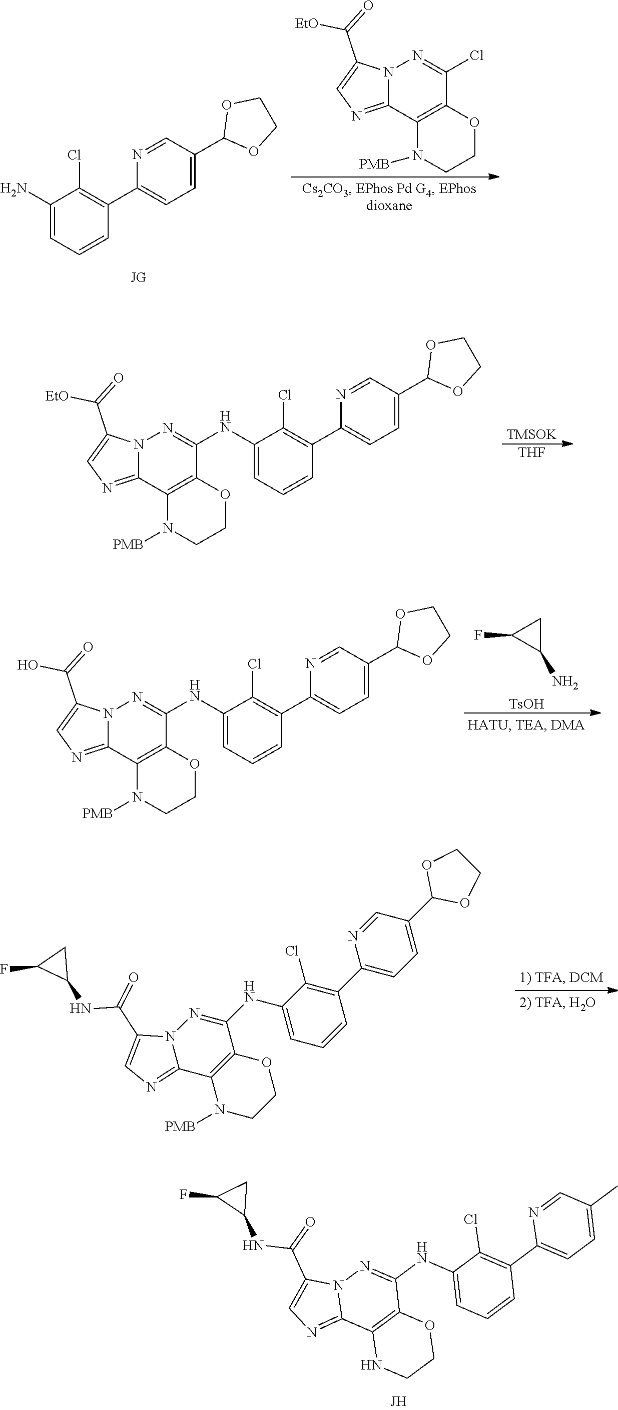

### Step 1—Ethyl 8-({2-chloro-3-[5-(1,3-dioxolan-2-yl)pyridin-2-yl]phenyl}amino)-13-[(4-methoxyphenyl)methyl]-10-oxa-3,6,7,13-tetraazatricyclo[7.4.0.0\hat{ }{2,6}]trideca-1(9),2,4,7-tetraene-5-carboxylate

To a stirred mixture of 2-chloro-3-[5-(1,3-dioxolan-2-yl)pyridin-2-yl]aniline (470 mg, 1.7 mmol, Intermediate JG) and ethyl 8-chloro-13-[(4-methoxyphenyl)methyl]-10-oxa-3,6,7,13-tetraazatricyclo[7.4.0.0\hat{ }{2,6}]trideca-1(9),2,4,7-tetraene-5-carboxylate (684.21 mg, 1.698 mmol, synthesized via Steps 1-3 of Intermediate JE) in dioxane (10 mL) were added Cs₂CO₃ (1.66 g, 5.09 mmol), EPhos Pd G4 (90.84 mg, 0.170 mmol) and EPhos (90.84 mg, 0.170 mmol) at rt under nitrogen atmosphere. The resulting mixture was then stirred for 2 h at 100° C. under nitrogen atmosphere. On completion, the mixture was cooled to rt and filtered, and the filter cake was washed with DCM (2×5 mL). The filtrate was concentrated under reduced pressure. The residue was purified by reverse flash chromatography (Column: WelFlash™ C18-I, 20-40 um, 330 g; Eluent A: Water (plus 10 mmol/L NH₄HCO₃); Eluent B: ACN; Gradient: 30%-50% B in 30 min; Flow rate: 80 mL/min; Detector: 220/254 nm; desired fractions were collected at 48% B) and concentrated under reduced pressure to afford the title compound (440 mg, 40% yield) as a yellow solid. LC/MS (ESI, m/z): [(M+H)]⁺=643.2.

### Step 2—8-({2-Chloro-3-[5-(1,3-dioxolan-2-yl)pyridin-2-yl]phenyl}amino)-13-[(4-methoxyphenyl)methyl]-10-oxa-3,6,7,13-tetraazatricyclo[7.4.0.0\hat{ }{2,6}]trideca-1(9),2,4,7-tetraene-5-carboxylic Acid

To a stirred solution of ethyl 8-({2-chloro-3-[5-(1,3-dioxolan-2-yl)pyridin-2-yl]phenyl}amino)-13-[(4-methoxyphenyl)methyl]-10-oxa-3,6,7,13-tetraazatricyclo[7.4.0.0\hat{ }{2,6}]trideca-1(9),2,4,7-tetraene-5-carboxylate (440 mg, 0.68 mmol) in THF (5 mL) was added trimethyl(potassiooxy)silane (175.54 mg, 1.368 mmol) at 0° C. The resulting mixture was stirred for 1 h at rt under nitrogen atmosphere. On completion, the mixture was concentrated under reduced pressure. The residue was purified by reverse flash chromatography (Column: WelFlash™ C18-I, 20-40 um, 330 g; Eluent A: Water (plus 10 mmol/L NH₄HCO₃); Eluent B: ACN; Gradient: 20%-40% B in 30 min; Flow rate: 80 mL/min; Detector: 220/254 nm; desired fractions were collected at 25% B) and concentrated under reduced pressure to afford the title compound (400 mg, 95% yield) as a yellow solid. LC/MS (ESI, m/z): [(M+H)]⁺=615.3.

### Step 3—8-({2-Chloro-3-[5-(1,3-dioxolan-2-yl)pyridin-2-yl]phenyl}amino)-N-[(1R,2S)-2-fluorocyclopropyl]-13-[(4-methoxyphenyl)methyl]-10-oxa-3,6,7,13-tetraazatricyclo[7.4.0.0\hat{ }{2,6}]trideca-1(9),2,4,7-tetraene-5-carboxamide

To a stirred solution of 8-({2-chloro-3-[5-(1,3-dioxolan-2-yl)pyridin-2-yl]phenyl}amino)-13-[(4-methoxyphenyl)methyl]-10-oxa-3,6,7,13-tetraazatricyclo[7.4.0.0\hat{ }{2,6}]trideca-1(9),2,4,7-tetraene-5-carboxylic acid (400 mg, 0.7 mmol) and (1R,2S)-2-fluorocyclopropan-1-amine; para-toluene sulfonate (160.82 mg, 0.650 mmol) in DMF (4 mL) were added DIEA (252.17 mg, 1.950 mmol) and HATU (370.94 mg, 0.975 mmol). The resulting mixture was stirred for 1 h at rt under nitrogen atmosphere. On completion, the resulting mixture was concentrated under reduced pressure. The residue was purified by reverse flash chromatography (Column: WelFlash™ C18-I, 20-40 um, 330 g; Eluent A: Water (plus 10 mmol/L FA); Eluent B: ACN; Gradient: 30%-50% B in 30 min; Flow rate: 80 mL/min; Detector: 220/254 nm; desired fractions were collected at 32% B) and concentrated under reduced pressure to afford the title compound (200 mg, 46% yield) as a yellow oil. LC/MS (ESI, m/z): [(M+H)]⁺=672.3.

### Step 4—8-{[2-Chloro-4′-(1,3-dioxolan-2-yl)-[1,1′-biphenyl]-3-yl]amino}-N-[(1R,2S)-2-fluorocyclopropyl]-10-oxa-3,6,7,13-tetraazatricyclo[7.4.0.0\hat{ }{2,6}]trideca-1(9),2,4,7-tetraene-5-carboxamide

To a stirred solution of 8-({2-chloro-3-[5-(1,3-dioxolan-2-yl)pyridin-2-yl]phenyl}amino)-N-[(1R,2S)-2-fluorocyclopropyl]-13-[(4-methoxyphenyl)methyl]-10-oxa-3,6,7,13-tetraazatricyclo[7.4.0.0\hat{ }{2,6}]trideca-1(9),2,4,7-tetraene-5-carboxamide (200 mg, 0.3 mmol) in DCM (6 mL) was added TFA (6 mL) dropwise at rt and the mixture was stirred for 1 h at rt under nitrogen atmosphere. On completion, the reaction mixture was concentrated under reduced pressure. Then the residue was dissolved in TFA (5 mL) and water (5 mL). The reaction mixture was stirred for 2 h at 50° C. On completion, the mixture was concentrated under reduced pressure. The residue was purified by reverse flash chromatography (Column: WelFlash™ C18-I, 20-40 um, 330 g; Eluent A: Water (plus 10 mmol/L NH₄HCO₃); Eluent B: ACN; Gradient: 50%-80% B in 30 min; Flow rate: 80 mL/min; Detector: 220/254 nm; desired fractions were collected at 75% B) and concentrated under reduced pressure to afford the title compound (100 mg, 61% yield) as a yellow solid. LC/MS (ESI, m/z): [(M+H)]⁺=508.3.

### Example 1.255. 1-(5-(4-(azetidin-3-yl)piperazine-1-carbonyl)-2-chlorophenyl)dihydropyrimidine-2,4(1H,3H)-dione (Intermediate JI)

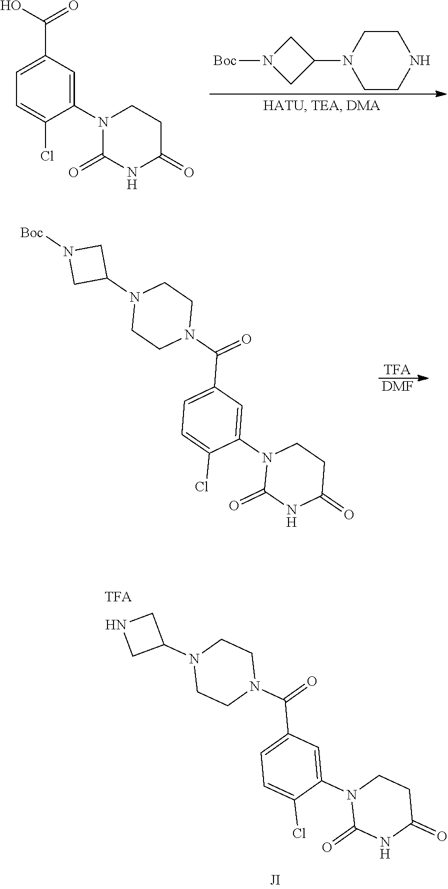

### Step 1—Tert-butyl 3-{4-[4-chloro-3-(2,4-dioxo-1,3-diazinan-1-yl)benzoyl]piperazin-1-yl}azetidine-1-carboxylate

To a stirred solution of 4-chloro-3-(2,4-dioxo-1,3-diazinan-1-yl)benzoic acid (500 mg, 2 mmol, synthesized via Steps 1-3 of Intermediate IO) and tert-butyl 3-(piperazin-1-yl)azetidine-1-carboxylate (539.0 mg, 2.233 mmol, CAS #178311-48-3) in DMA (10 mL) were added HATU (849.21 mg, 2.233 mmol) and TEA (0.78 mL, 5.583 mmol) in portions at rt under nitrogen atmosphere. The resulting mixture was stirred for 1 h at rt under nitrogen atmosphere. On completion, the mixture was concentrated under reduced pressure. The residue was purified by reverse phase Flash chromatography (Column: WelFlash™ C18-I, 20-40 um, 330 g; Eluent A: Water (10 mmol/L NH₄HCO₃); Eluent B: ACN; Gradient: 30%-60% B in 30 min; Flow rate: 80 mL/min; Detector: 254 nm; desired fractions were collected at 50% B) and concentrated under reduced pressure to afford the title compound (700 mg, 77% yield) as a light yellow solid. LC/MS (ESI, m/z): [(M+H)]⁺=492.2.

### Step 2—1-{5-[4-(Azetidin-3-yl)piperazine-1-carbonyl]-2-chlorophenyl}-1,3-diazinane-2,4-dione Trifluoroacetate

To a stirred solution of tert-butyl 3-{4-[4-chloro-3-(2,4-dioxo-1,3-diazinan-1-yl)benzoyl]piperazin-1-yl}azetidine-1-carboxylate (700 mg, 1 mmol) in DCM (10 mL) was added TFA (2 mL) dropwise at rt under nitrogen atmosphere. The resulting mixture was stirred for 1 h at rt under nitrogen atmosphere. On completion, the mixture was concentrated under reduced pressure. The residue was purified by trituration with ethyl ether (40 mL) to afford the title compound (600 mg, 86% yield) as a light yellow solid. LC/MS (ESI, m/z): [(M+H)]⁺=392.2.

### Example 1.256. N-((1R,2S)-2-fluorocyclopropyl)-6-(4-(4-formylpiperidin-1-yl)indolin-1-yl)-8-(methylamino)imidazo[1,2-b]pyridazine-3-carboxamide (Intermediate JJ)

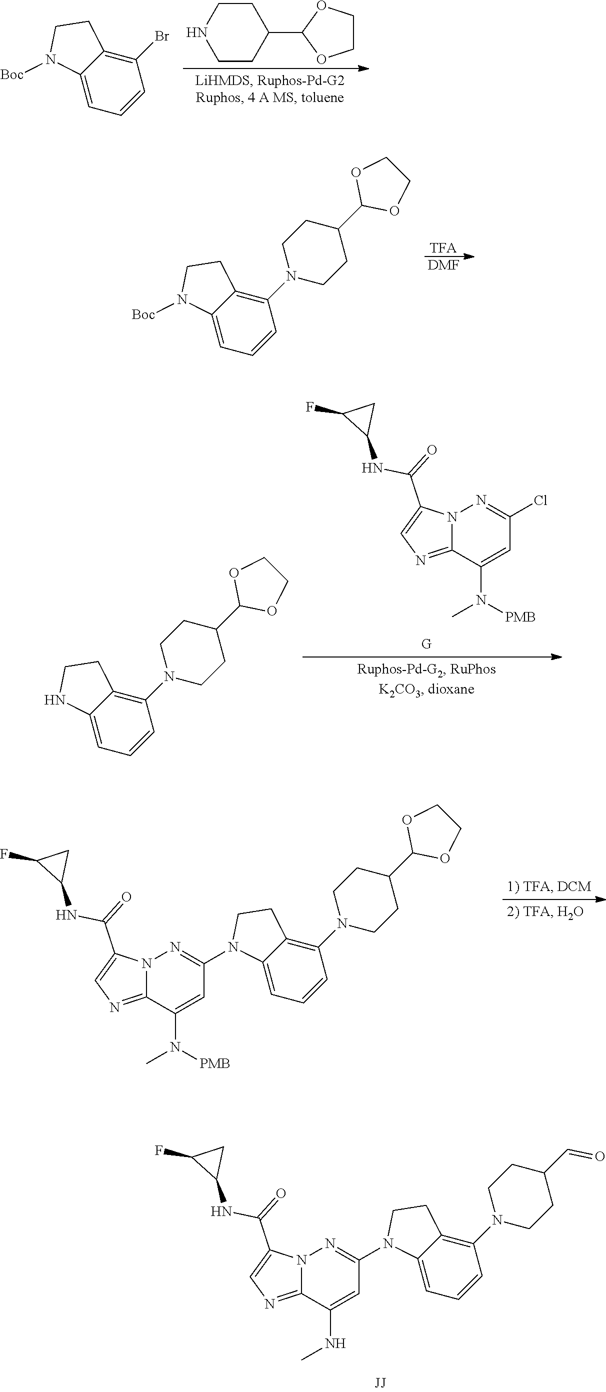

### Step 1—Tert-butyl 4-[4-(1,3-dioxolan-2-yl)piperidin-1-yl]-2,3-dihydroindole-1-carboxylate

To a stirred solution tert-butyl 4-bromo-2,3-dihydroindole-1-carboxylate (10.00 g, 33.54 mmol, CAS #885272-46-8) and LiHMDS (200 mL, 200 mmol) in toluene (100 mL) at 0° C. under nitrogen atmosphere. The resulting solution was stirred for 30 min at 0° C. under nitrogen atmosphere. Then added RuPhos (3.13 g, 6.71 mmol) and RuPhos-PdCl-2nd G (2.61 g, 3.35 mmol) to the reaction at rt under nitrogen atmosphere. The resulting solution was stirred for 2 h at 80° C. under nitrogen atmosphere. On completion, the reaction mixture was cooled to rt and concentrated under vacuum. The residue was purified by silica gel column chromatography, eluted with PE/EA (5:1˜1:1), to afford the title compound (9.00 g, 72% yield) as a brown oil. LC/MS (ESI, m/z): [(M+H)]⁺=375.3.

### Step 2—4-[4-(1,3-Dioxolan-2-yl)piperidin-1-yl]-2,3-dihydro-1H-indole

A solution of tert-butyl 4-[4-(1,3-dioxolan-2-yl)piperidin-1-yl]-2,3-dihydroindole-1-carboxylate (9.00 g, 24.0 mmol) in DCM (50 mL) and TFA (50 mL) was stirred for 2 h at rt under nitrogen atmosphere. On completion, the reaction was concentrated under vacuum. The residue was purified by trituration with diethyl ether (100 mL) to afford the title compound (6.50 g, 99% yield) as a yellow solid. LC/MS (ESI, m/z): [(M+H)]⁺=275.3.

### Step 3—6-{4-[4-(1,3-Dioxolan-2-yl)piperidin-1-yl]-2,3-dihydroindol-1-yl}-N-[(1R,2S)-2-fluorocyclopropyl]-8-{[(4-methoxyphenyl)methyl](methyl)amino}imidazo[1,2-b]pyridazine-3-carboxamide

To a stirred mixture of 4-[4-(1,3-dioxolan-2-yl)piperidin-1-yl]-2,3-dihydro-1H-indole (1.00 g, 3.65 mmol) and 6-chloro-N-[(1R,2S)-2-fluorocyclopropyl]-8-{[(4-methoxyphenyl)methyl](methyl)amino}imidazo[1,2-b]pyridazine-3-carboxamide (1.62 g, 4.01 mmol, Intermediate G) in dioxane (12 mL) was added RuPhos Palladacycle Gen. 3 (0.30 g, 0.37 mmol), RuPhos (0.34 g, 0.73 mmol) and K₂CO₃ (1.51 g, 10.9 mmol) in portions at rt under nitrogen atmosphere. The resulting mixture was stirred for 2 h at 100° C. under nitrogen atmosphere. On completion, the reaction mixture was concentrated under vacuum. The residue was purified by silica gel column chromatography, eluted with PE/EA (5:1˜1:8), to afford the title compound (1.20 g, 51% yield) as a yellow solid. LC/MS (ESI, m/z): [(M+H)]⁺=642.4.

### Step 4—N-[(1R,2S)-2-fluorocyclopropyl]-6-[4-(4-formylpiperidin-1-yl)-2,3-dihydroindol-1-yl]-8-(methylamino)imidazo[1,2-b]pyridazine-3-carboxamide

To a stirred mixture of 6-{4-[4-(1,3-dioxolan-2-yl)piperidin-1-yl]-2,3-dihydroindol-1-yl}-N-[(1R,2S)-2-fluorocyclopropyl]-8-{[(4-methoxyphenyl)methyl](methyl)amino}imidazo[1,2-b]pyridazine-3-carboxamide (600 mg, 0.9 mmol) in DCM (5 mL) was added TFA (5 mL) dropwise at rt. The resulting solution was stirred for 1 h at rt. After deprotection of the PMB, the resulting solution was concentrated under vacuum. The residue was re-dissolved in 2M aq. TFA (10 mL) and the resulting mixture was stirred for 16 h at 50° C. under nitrogen atmosphere. On completion, the mixture was concentrated under vacuum. The residue was purified by trituration with diethyl ether (15 mL) to afford the title compound (400 mg, 90% yield) as a white solid. LC/MS (ESI, m/z): [(M+H)]⁺=478.3.

### Example 1.257. N-[(1R,2S)-2-fluorocyclopropyl]-6-{[3-(4-formylpiperidin-1-yl)phenyl]amino}-8-(methylamino)imidazo[1,2-b]pyridazine-3-carboxamide (Intermediate JK)

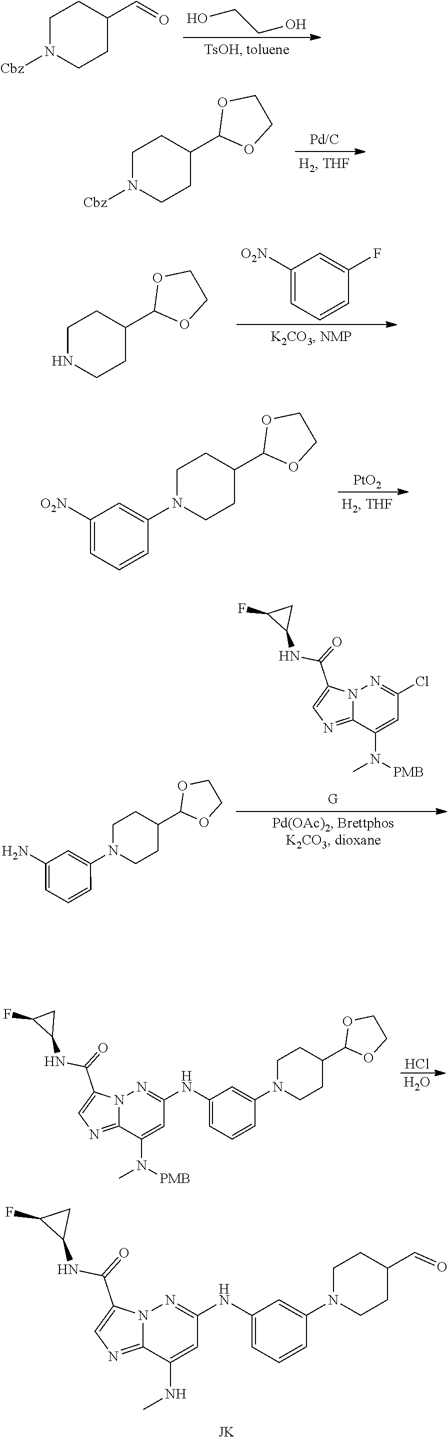

### Step 1—Benzyl 4-(1,3-dioxolan-2-yl)piperidine-1-carboxylate

To a stirred solution of benzyl 4-formylpiperidine-1-carboxylate (10 g, 40 mmol) in toluene (50 mL) and ethylene glycol (10 mL) was added TsOH (0.70 g, 4.044 mmol) at rt under nitrogen atmosphere. The resulting mixture was stirred for 2 h at 110° C. under nitrogen atmosphere. On completion, the mixture was cooled to rt and concentrated under reduced pressure. The residue was triturated by Et₂O to afford the title compound (10 g, 85% yield) as a light brown oil. LC/MS (ESI, m/z): [(M+H)]⁺=292.3.

### Step 2—4-(1,3-Dioxolan-2-yl)piperidine

To a solution of benzyl 4-(1,3-dioxolan-2-yl)piperidine-1-carboxylate (10 g, 34 mmol) in THF (100 mL) was added Pd/C (0.37 g, 3.43 mmol, 10 wt %) under nitrogen atmosphere. The reaction system was degassed under vacuum and purged with H₂ several times, then it was hydrogenated under H₂ balloon (˜1 atm) at 25° C. for 16 h. After completion of the reaction, Pd/C was filtered off through celite and the filter cake was washed with THF (3×100 mL). The corresponding filtrate was concentrated under reduced pressure to provide the title compound (6 g, 95% yield) as a light brown oil. LC/MS (ESI, m/z): [(M+H)]⁺=158.1.

### Step 3—4-(1,3-Dioxolan-2-yl)-1-(3-nitrophenyl)piperidine

To a stirred solution of 3-fluoronitrobenzene (17.95 g, 127.2 mmol) and 4-(1,3-dioxolan-2-yl)piperidine (10 g, 60 mmol) in NMP (60 mL) was added K₂CO₃ (35.16 g, 254.4 mmol) at rt under nitrogen atmosphere. The resulting mixture was stirred for 16 h at 110° C. under nitrogen atmosphere. On completion, the mixture was cooled to rt and concentrated under reduced pressure. The residue was purified by silica gel column chromatography, eluted with PE/EA (5:1), to afford the title compound (2 g, 11% yield) as a reddish brown semi-solid. LC/MS (ESI, m/z): [(M+H)]⁺=279.2.

### Step 4—3-[4-(1,3-Dioxolan-2-yl)piperidin-1-yl]aniline

To a solution of 4-(1,3-dioxolan-2-yl)-1-(3-nitrophenyl)piperidine (1.8 g, 6.5 mmol) in THF (20 mL) was added PtO₂ (0.15 g, 0.65 mmol) under nitrogen atmosphere. The reaction system was degassed under vacuum and purged with H₂ several times, then it was hydrogenated under H₂ balloon (˜1 atm) at 25° C. for 3 h. After completion, PtO₂ was filtered off through celite and the filter cake was washed with THF (3×20 mL). The corresponding filtrate was concentrated under reduced pressure to provide the title compound (1.6 g, quant. crude yield) as an off-white solid. LC/MS (ESI, m/z): [(M+H)]⁺=249.3.

### Step 5—6-({3-[4-(1,3-Dioxolan-2-yl)piperidin-1-yl]phenyl}amino)-N-[(1R,2S)-2-fluorocyclopropyl]-8-{[(4-methoxyphenyl)methyl](methyl)amino}imidazo[1,2-b]pyridazine-3-carboxamide

To a stirred solution of 3-[4-(1,3-dioxolan-2-yl)piperidin-1-yl]aniline (1.58 g, 6.39 mmol) and 6-chloro-N-[(1R,2S)-2-fluorocyclopropyl]-8-{[(4-methoxyphenyl)methyl](methyl)amino}imidazo[1,2-b]pyridazine-3-carboxamide (3.10 g, 7.66 mmol, Intermediate G) in 1,4-dioxane (20 mL) were added Pd(OAc)₂ (0.14 g, 0.64 mmol), Brettphos (0.34 g, 0.69 mmol) and K₂CO₃ (17.65 g, 127.7 mmol) in portions at rt. The resulting mixture was stirred for 16 h at 100° C. under nitrogen atmosphere. On completion, the mixture was cooled to rt and concentrated under reduced pressure. The residue was purified by silica gel column chromatography, eluted with PE/EA (1:1), to afford the title compound (1.9 g, 48% yield) as a white solid. LC/MS (ESI, m/z): [(M+H)]⁺=616.3.

### Step 6—N-[(1R,2S)-2-fluorocyclopropyl]-6-{[3-(4-formylpiperidin-1-yl)phenyl]amino}-8-(methylamino)imidazo[1,2-b]pyridazine-3-carboxamide

To a stirred solution of 6-({3-[4-(1,3-dioxolan-2-yl)piperidin-1-yl]phenyl}amino)-N-[(1R,2S)-2-fluorocyclopropyl]-8-{[(4-methoxyphenyl)methyl](methyl)amino}imidazo[1,2-b]pyridazine-3-carboxamide (2 g, 3 mmol) in 1,4-dioxane (15 mL) was added HCl (6 M) (15 mL) at rt under nitrogen atmosphere. The resulting mixture was stirred for 16 h at 50° C. under nitrogen atmosphere. On completion, the mixture was concentrated under reduced pressure. The residue was purified by reverse flash chromatography (Column: WelFlash™ C18-I, 20-40 μm, 330 g; Eluent A: Water (plus 10 mmol/L FA); Eluent B: ACN; Gradient: 50%-90% B in 30 min; Flow rate: 60 mL/min; Detector: 220/254 nm; desired fractions were collected at 88% B) and concentrated under reduced pressure to afford the title compound (120 mg, 8% yield) as an off-white solid. LC/MS (ESI, m/z): [(M+H)]⁺=452.3.

### Example. 1.258. 6-{[3-(5-formylpyridin-2-yl)-2-methoxyphenyl]amino}-N-isopropyl-8-(methylamino)imidazo[1,2-b]pyridazine-3-carboxamide (Intermediate JL)

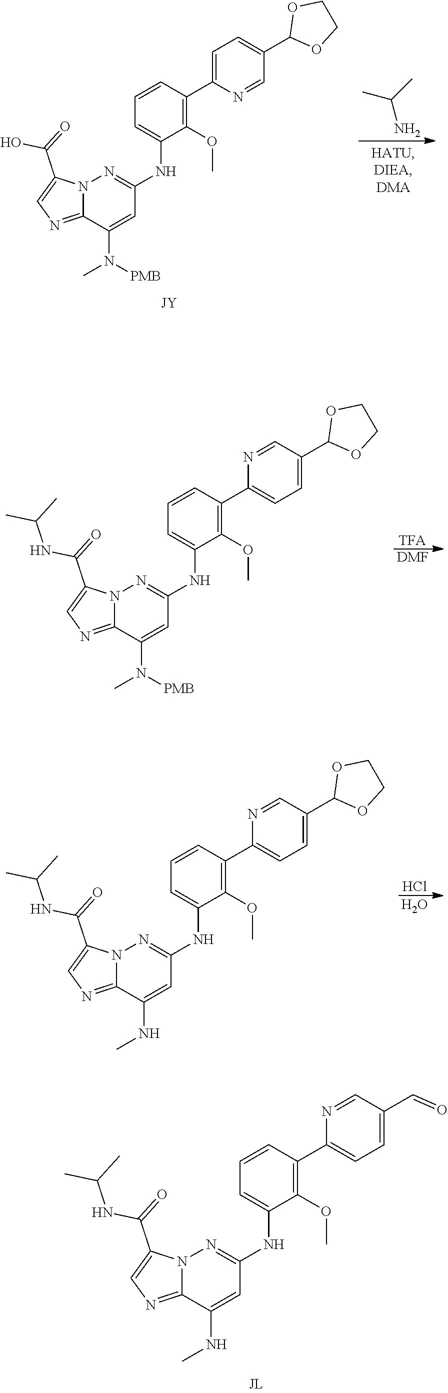

### Step 1—6-({3-[5-(1,3-Dioxolan-2-yl)pyridin-2-yl]-2-methoxyphenyl}amino)-N-isopropyl-8-{[(4-methoxyphenyl)methyl](methyl)amino}imidazo[1,2-b]pyridazine-3-carboxamide

To a stirred solution of 6-({3-[5-(1,3-dioxolan-2-yl)pyridin-2-yl]-2-methoxyphenyl}amino)-8-{[(4-methoxyphenyl)methyl](methyl)amino}imidazo[1,2-b]pyridazine-3-carboxylic acid (1 g, 2 mmol, Intermediate JY) and isopropylamine (0.15 g, 2.6 mmol) in DMF (10 mL) were added DIEA (0.44 g, 3.4 mmol) and HATU (0.98 g, 2.6 mmol) at 0° C. under nitrogen atmosphere. The resulting mixture was then stirred for 1 h rt under nitrogen atmosphere. On completion, the mixture was concentrated under reduced pressure. The residue was purified by reverse flash chromatography (Column: WelFlash™ C18-I, 20-40 μm, 330 g; Eluent A: Water (plus 10 mmol/L FA); Eluent B: ACN; Gradient: 40%-60% B in 30 min; Flow rate: 80 mL/min; Detector: 220/254 nm; desired fractions were collected at 54% B) and concentrated under reduced pressure to afford the title compound (800 mg, 75% yield) as an off-white solid. LC/MS (ESI, m/z): [(M+H)]⁺=624.3.

### Step 2—6-({3-[5-(1,3-dioxolan-2-yl)pyridin-2-yl]-2-methoxyphenyl}amino)-N-isopropyl-8-(methylamino)imidazo[1,2-b]pyridazine-3-carboxamide

To a solution of 6-({3-[5-(1,3-dioxolan-2-yl)pyridin-2-yl]-2-methoxyphenyl}amino)-N-isopropyl-8-{[(4-methoxyphenyl)methyl](methyl)amino}imidazo[1,2-b]pyridazine-3-carboxamide (800 mg, 1 mmol) in DCM (10 mL) was added TFA (5 mL) dropwise at rt. The reaction mixture was then stirred for 2 h at rt under nitrogen atmosphere. On completion, the mixture was concentrated under reduced pressure. The residue was triturated with Et₂O to afford the title compound (760 mg) as a brown solid. LC/MS (ESI, m/z): [(M+H)]⁺=504.1.

### Step 3—6-{[3-(5-formylpyridin-2-yl)-2-methoxyphenyl]amino}-N-isopropyl-8-(methylamino)imidazo[1,2-b]pyridazine-3-carboxamide

To a stirred solution of 6-({3-[5-(1,3-dioxolan-2-yl)pyridin-2-yl]-2-methoxyphenyl}amino)-N-isopropyl-8-(methylamino)imidazo[1,2-b]pyridazine-3-carboxamide (760 mg, 1.3 mmol) in H₂O (10 mL) was added a solution of 6 M HCl (5 mL) at 0° C. under nitrogen atmosphere. The resulting mixture was then stirred for 2 h at 50° C. under nitrogen atmosphere. On completion, the mixture was cooled to rt and concentrated under reduced pressure. The residue was purified by reverse phase flash chromatography (Column: WelFlash™ C18-I, 20-40 μm, 330 g; Eluent A: Water (plus 10 mmol/L FA); Eluent B: ACN; Gradient: 50%-70% B in 20 min; Flow rate: 80 mL/min; Detector: 220/254 nm; desired fractions were collected at 65% B) and concentrated under reduced pressure to afford the title compound (200 mg, 34% two steps) as an off-white solid. ¹H NMR (400 MHz, DMSO-d₆) δ 10.17 (s, 1H), 9.24-9.18 (m, 1H), 8.59 (s, 1H), 8.52 (d, J=8.0 Hz, 1H), 8.34 (dd, J=8.2, 2.2 Hz, 1H), 8.08 (d, J=8.2 Hz, 1H), 7.84 (dd, J=7.9, 1.7 Hz, 1H), 7.80 (s, 1H), 7.58 (dd, J=7.8, 1.7 Hz, 1H), 7.49-7.40 (m, 1H), 7.35-7.27 (m, 1H), 6.10 (s, 1H), 4.12-3.99 (m, 1H), 3.56 (s, 3H), 2.91 (d, J=4.9 Hz, 3H), 0.99 (d, J=6.6 Hz, 6H). LC/MS (ESI, m/z): [(M+H)]⁺=460.3.

### Example 1.259. N-cyclopropyl-6-((3-formylphenyl)amino)-8-(methylamino)imidazo[1,2-b]pyridazine-3-carboxamide (Intermediate JM)

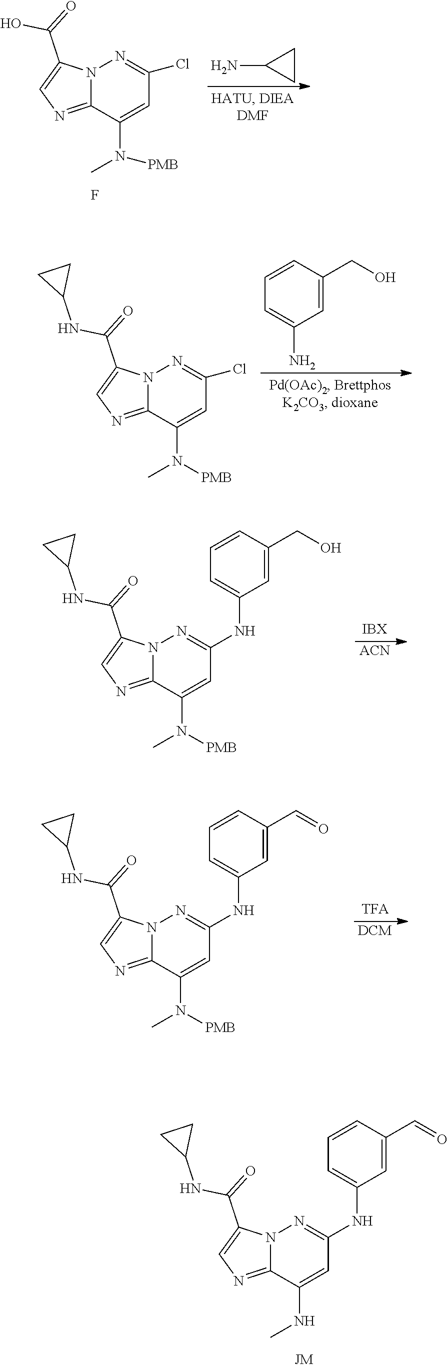

### Step 1—6-Chloro-N-cyclopropyl-8-{[(4-methoxyphenyl)methyl](methyl)amino}imidazo[1,2-b]pyridazine-3-carboxamide

To a stirred mixture of 6-chloro-8-{[(4-methoxyphenyl)methyl](methyl)amino}imidazo[1,2-b]pyridazine-3-carboxylic acid (30 g, 90 mmol, Intermediate F) and HATU (49.34 g, 129.769 mmol) in DMF (300 mL) were added aminocyclopropane (5.93 g, 104 mmol) and DIEA (33.54 g, 259.5 mmol) at rt. The resulting mixture was stirred for 2 h at rt. On completion, the mixture was diluted with water (500 mL) and extracted with EtOAc (3×300 mL). The combined organic layers were washed with brine (3×1000 mL), and dried over anhydrous Na₂SO₄. After filtration, the filtrate was concentrated under reduced pressure. The residue was purified by silica gel column chromatography, eluted with CH₂Cl₂/EA (50:1), to afford the title compound (22 g, 66% yield) as a grey solid. LC/MS (ESI, m/z): [(M+H)]⁺=386.2.

### Step 2—N-cyclopropyl-6-{[3-(hydroxymethyl)phenyl]amino}-8-{[(4-methoxyphenyl)methyl](methyl)amino}imidazo[1,2-b]pyridazine-3-carboxamide

To a stirred mixture of 6-chloro-N-cyclopropyl-8-{[(4-methoxyphenyl)methyl](methyl)amino}imidazo[1,2-b]pyridazine-3-carboxamide (27 g, 70 mmol) and (3-aminophenyl)methanol (10.34 g, 83.97 mmol) in dioxane (500 mL) were added Pd(OAc)₂ (1.57 g, 7.00 mmol), BrettPhos (3.76 g, 7.00 mmol) and K₂CO₃ (19.34 g, 140.0 mmol) at rt under nitrogen atmosphere. The resulting mixture was stirred for 1 h at 80° C. under nitrogen atmosphere. On completion, the mixture was cooled to rt and concentrated under reduced pressure. The residue was purified by silica gel column chromatography, eluted with CH₂Cl₂/EtOAc (50:1), to afford the title compound (27 g, 82% yield) as a light yellow solid. LC/MS (ESI, m/z): [(M+H)]⁺=473.3.

### Step 3—N-cyclopropyl-6-[(3-formylphenyl)amino]-8-{[(4-methoxyphenyl)methyl](methyl)amino}imidazo[1,2-b]pyridazine-3-carboxamide

To a solution of N-cyclopropyl-6-{[3-(hydroxymethyl)phenyl]amino}-8-{[(4-methoxyphenyl)methyl](methyl)amino}imidazo[1,2-b]pyridazine-3-carboxamide (8.5 g, 18 mmol) ACN (150 mL) and DMSO (5 mL) was added IBX (10.07 g, 36.00 mmol) in portions at rt. The resulting mixture was then stirred for 1 h at 90° C. On completion, the mixture was cooled to rt and was filtered, then the filter cake was washed with DCM:MeOH (10:1) (3×200 mL). The collected solid was dried under vacuum to afford the title compound (5 g, 59% yield) as a grey solid. LC/MS (ESI, m/z): [(M+H)]⁺=471.3.

### Step 4—N-cyclopropyl-6-[(3-formylphenyl)amino]-8-(methylamino)imidazo[1,2-b]pyridazine-3-carboxamide

A solution of N-cyclopropyl-6-[(3-formylphenyl)amino]-8-{[(4-methoxyphenyl)methyl](methyl)amino}imidazo[1,2-b]pyridazine-3-carboxamide (19 g, 40 mmol) in 20% TFA DCM solution (250 mL) was stirred for 2 h at rt. The resulting mixture was concentrated under reduced pressure. The residue was purified by reverse flash chromatography (column, C18 silica gel; mobile phase, MeCN in Water (10 mmol/L NH₄HCO₃), 5% to 50% gradient in 40 min; detector, UV 254 nm) to afford the title compound (5.7 g, 40% yield) as a grey powder. LC/MS (ESI, m/z): [(M+H)]⁺=251.2.

### Example 1.260. 6-({3-[5-(1,3-dioxolan-2-yl)pyridin-2-yl]phenyl}amino)-8-{[(4-methoxyphenyl)methyl](methyl)amino}imidazo[1,2-b]pyridazine-3-carboxylate (Intermediate JN)

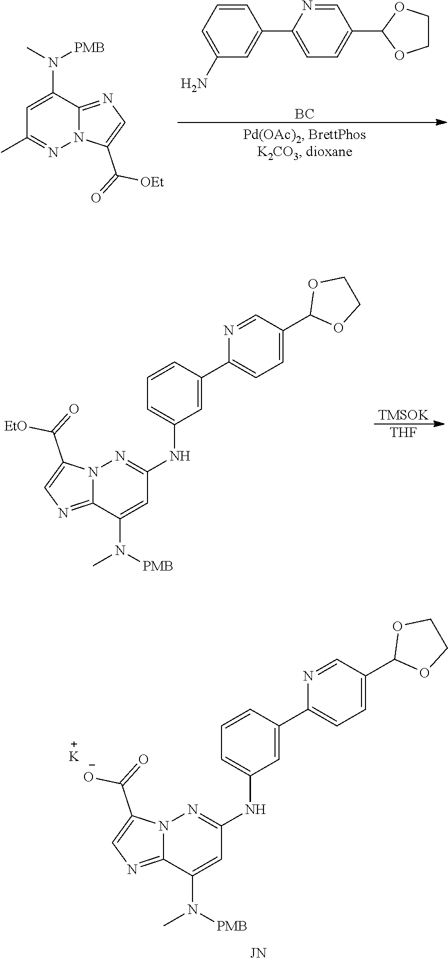

### Step 1—6-({3-[5-(1,3-dioxolan-2-yl)pyridin-2-yl]phenyl}amino)-8-{[(4-methoxyphenyl)methyl](methyl)amino}imidazo[1,2-b]pyridazine-3-carboxylate

To a stirred mixture of ethyl 6-chloro-8-{[(4-methoxyphenyl)methyl](methyl)amino}imidazo[1,2-b]pyridazine-3-carboxylate (7.0 g, 19 mmol, synthesized via Steps 1-2 of Intermediate F), 3-[5-(1,3-dioxolan-2-yl)pyridin-2-yl]aniline (6.79 g, 28.0 mmol, Intermediate BC) and K₂CO₃ (5.16 g, 37.4 mmol) in dioxane (120 mL) were added Pd(OAc)₂ (838.55 mg, 3.735 mmol) and BrettPhos (2.00 g, 3.74 mmol) in portions at rt under nitrogen atmosphere. The resulting mixture was stirred for 3 h at 100° C. under nitrogen atmosphere. After completion of reaction, the mixture was cooled to rt and concentrated under reduced pressure. The residue was dissolved in MeCN (30 mL), water (30 mL) and DMSO (10 mL). The precipitated solids were collected by filtration and washed with water (2×10 mL). The resulting solid was dried under infrared light to afford the title compound (7 g, 65% yield) as a brown solid. LC/MS (ESI, m/z): [(M+H)]⁺=581.3.

### Step 2—Potassium 6-({3-[5-(1,3-dioxolan-2-yl)pyridin-2-yl]phenyl}amino)-8-{[(4-methoxyphenyl)methyl](methyl)amino}imidazo[1,2-b]pyridazine-3-carboxylate

To a stirred solution of ethyl 6-({3-[5-(1,3-dioxolan-2-yl)pyridin-2-yl]phenyl}amino)-8-{[(4-methoxyphenyl)methyl](methyl)amino}imidazo[1,2-b]pyridazine-3-carboxylate (7 g, 10 mmol) in THF (100 mL) was added potassium trimethylsilanolate (3.09 g, 24.1 mmol) in portions at 0° C. under nitrogen atmosphere. The resulting mixture was stirred for 2 h at rt under nitrogen atmosphere. On completion, the mixture was concentrated under reduced pressure. The residue was purified by trituration with Et₂O (100 mL). The precipitated solids were collected by filtration and washed with Et₂O (2×10 mL), then dried under vacuum to afford the title compound (6 g, 84% yield) as an off-white solid. LC/MS (ESI, m/z): [(M+H)]⁺=553.3.

### Example 1.261. N-cyclopropyl-6-{[3-(5-formylpyridin-2-yl)phenyl]amino}-8-(methylamino)imidazo[1,2-b]pyridazine-3-carboxamide (Intermediate JO)

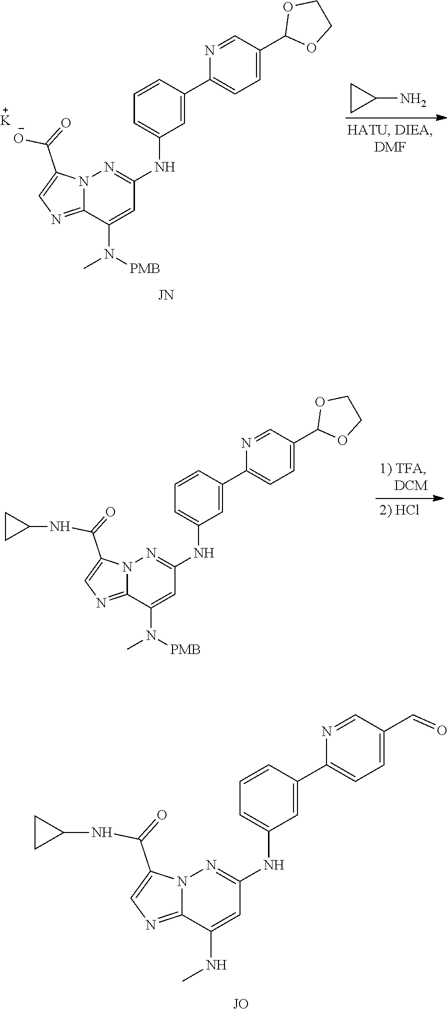

### Step 1—6-({3-[5-(1,3-Dioxolan-2-yl)pyridin-2-yl]phenyl}amino)-N-(2-methoxycyclopropyl)-8-{[(4-methoxyphenyl)methyl](methyl)amino}imidazo[1,2-b]pyridazine-3-carboxamide

To a stirred solution of potassium 6-({3-[5-(1,3-dioxolan-2-yl)pyridin-2-yl]phenyl}amino)-8-{[(4-methoxyphenyl)methyl](methyl)amino}imidazo[1,2-b]pyridazine-3-carboxylate (400 mg, 0.6 mmol, Intermediate IN) and aminocyclopropane (55.10 mg, 0.965 mmol) in DMF (6 mL) were added HATU (366.95 mg, 0.965 mmol) and DIEA (0.22 mL, 1.3 mmol) in portions at rt under nitrogen atmosphere. The resulting mixture was stirred for 2 h at rt under nitrogen atmosphere. After completion of reaction, the reaction mixture was added to water (20 mL) dropwise. The precipitated solids were collected by filtration. The filter cake was washed with MeCN (10 mL) and dried under vacuum to afford the title compound (300 mg, 75% yield) as a white solid. LC/MS (ESI, m/z): [(M+H)]⁺=592.3.

### Step 2—N-cyclopropyl-6-{[3-(5-formylpyridin-2-yl)phenyl]amino}-8-(methylamino)imidazo[1,2-b]pyridazine-3-carboxamide

To a stirred solution of N-cyclopropyl-6-({3-[5-(1,3-dioxolan-2-yl)pyridin-2-yl]phenyl}amino)-8-{[(4-methoxyphenyl)methyl](methyl)amino}imidazo[1,2-b]pyridazine-3-carboxamide (300 mg, 0.5 mmol) in DCM (6 mL) was added TFA (6 mL) dropwise. The resulting mixture was stirred for 2 h at rt. After fully de-protection of PMB group, the mixture was concentrated under vacuum. The residue was then dissolved in 1 M aq. HCl (10 mL) and stirred for 16 h at 50° C. under nitrogen atmosphere. The resulting mixture was concentrated under reduced pressure. The residue was purified by trituration with Et₂O (5 mL). The crude product was purified by Prep-HPLC (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 27% B to 37% B in 10 min, 37% B; Wave Length: 254 nm; RT1 (min): 11.2) to afford the title compound (104 mg, 48% yield) as a light yellow solid. ¹H NMR (400 MHz, DMSO-d₆) δ 10.15 (s, 1H), 9.32 (s, 1H), 9.17 (d, J=2.0 Hz, 1H), 8.71 (d, J=3.9 Hz, 1H), 8.34 (dt, J=6.4, 2.2 Hz, 2H), 8.25 (d, J=8.3 Hz, 1H), 7.89-7.80 (m, 2H), 7.61 (dd, J=7.9, 2.1 Hz, 1H), 7.53 (dd, J=8.6, 6.7 Hz, 2H), 5.80 (s, 1H), 2.90 (d, J=4.8 Hz, 3H), 2.69-2.63 (m, 1H), 0.51-0.45 (m, 2H), 0.25-0.16 (m, 2H). LC/MS (ESI, m/z): [(M+H)]⁺=428.2.

### Example 1.262. 3-(3-methyl-2-oxo-5-(4-(piperidin-4-yl)phenyl)-2,3-dihydro-1H-benzo[d]imidazol-1-yl)piperidine-2,6-dione (Intermediate JP)

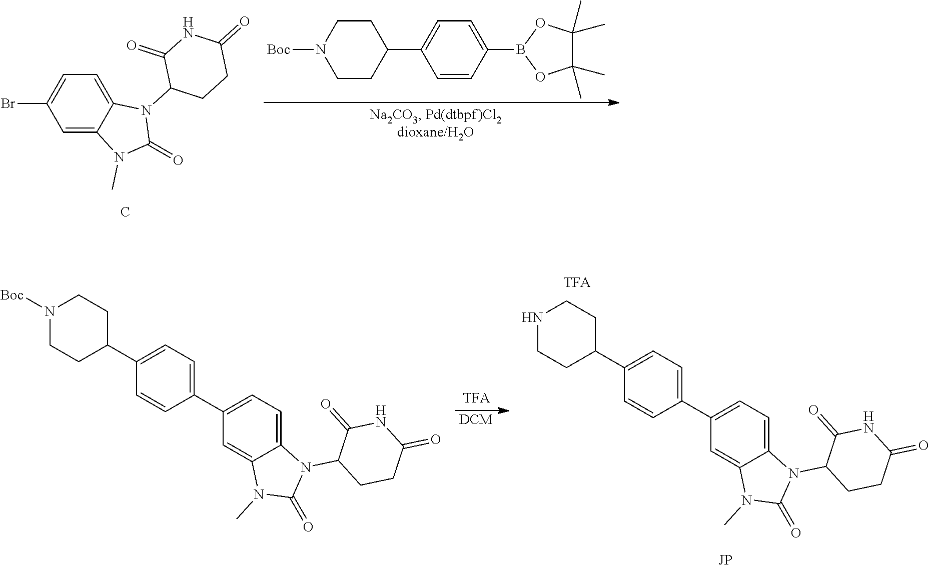

### Step 1—4-{4-[1-(2,6-Dioxopiperidin-3-yl)-3-methyl-2-oxo-1,3-benzodiazol-5-yl]phenyl}piperidine-1-carboxylate

To a stirred solution of tert-butyl 4-[4-(4,4,5,5-tetramethyl-1,3,2-dioxaborolan-2-yl)phenyl]piperidine-1-carboxylate (CAS #956136-85-9) (1.5 g, 3.9 mmol) and 3-(5-bromo-3-methyl-2-oxo-1,3-benzodiazol-1-yl)piperidine-2,6-dione (1.57 g, 4.65 mmol, Intermediate C) in 1,4-dioxane (20 mL) were added Pd(Dppp)Cl₂ (0.23 g, 0.39 mmol) and K₃PO₄ (2.47 g, 11.6 mmol) in portions at rt under nitrogen atmosphere. The resulting mixture was stirred for 2 h at 90° C. under nitrogen atmosphere. On completion, the mixture was cooled rt and purified by reverse phase Flash chromatography (Column: WelFlash™ C18-I, 20-40 um, 330 g; Eluent A: Water (plus 10 mmol/L NH₄HCO₃); Eluent B: ACN; Gradient: 35%-65% B in 25 min; Flow rate: 80 mL/min; Detector: 220/254 nm; desired fractions were collected at 50% B) and concentrated under reduced pressure to afford crude product as a brown solid. The crude product was repurified by Prep-TLC (CH₂Cl₂/EtOAc 2:3) to afford the title compound (160 mg, 8% yield) as a brown solid. LC/MS (ESI, m/z): [(M+H)]⁺=519.3.

### Step 2—3-{3-Methyl-2-oxo-5-[4-(piperidin-4-yl)phenyl]-1,3-benzodiazol-1-yl}piperidine-2,6-dione Trifluoroacetate

To a stirred solution of tert-butyl 4-{4-[1-(2,6-dioxopiperidin-3-yl)-3-methyl-2-oxo-1,3-benzodiazol-5-yl]phenyl}piperidine-1-carboxylate (160 mg, 0.31 mmol) in DCM (3 mL) was added TFA (1 mL) dropwise at rt under nitrogen atmosphere. The resulting mixture was stirred for 2 h at rt under nitrogen atmosphere. On completion, the resulting mixture was concentrated under reduced pressure. The residue was purified by trituration with EtOAc (6 mL) to afford the title compound (130 mg, 82% yield) as a brown solid. LC/MS (ESI, m/z): [(M+H)]⁺=419.2.

### Example 1.263. 6-[1-(3-{[(1R,2S)-2-fluorocyclopropyl]carbamoyl}-8-(methylamino)imidazo[1,2-b]pyridazin-6-yl)-2,3-dihydroindol-4-yl]pyridine-3-carboxylic Acid (Intermediate JQ)

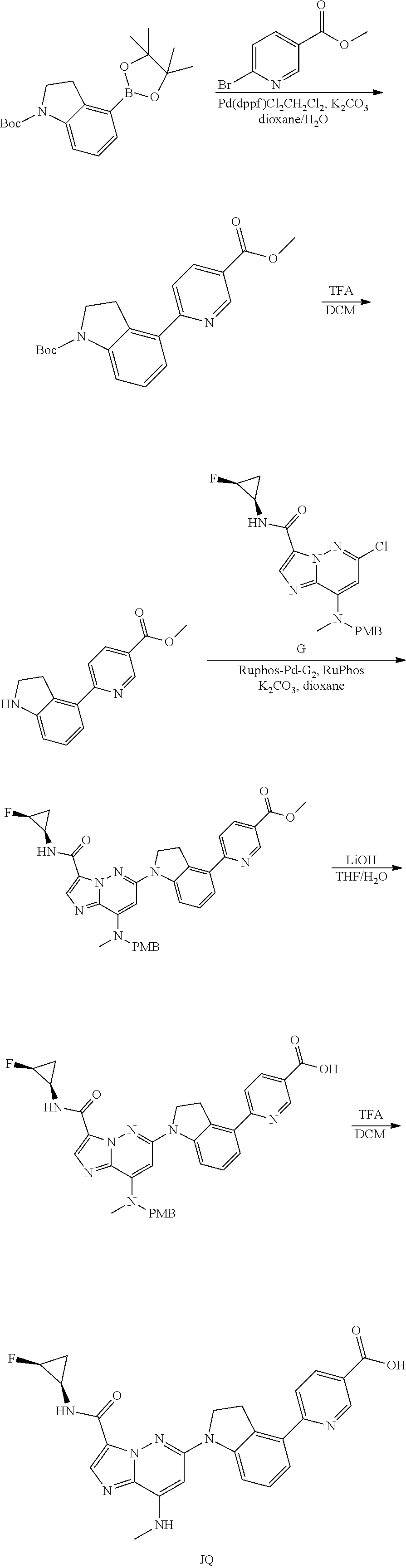

### Step 1—Tert-butyl 4-[5-(methoxycarbonyl)pyridin-2-yl]-2,3-dihydroindole-1-carboxylate

To a stirred mixture of tert-butyl 4-(4,4,5,5-tetramethyl-1,3,2-dioxaborolan-2-yl)-2,3-dihydroindole-1-carboxylate (10.00 g, 28.97 mmol, CAS #1235451-62-3) and methyl 6-bromopyridine-3-carboxylate (9.39 g, 43.5 mmol, CAS #26218-78-0) in dioxane (100 mL) and H₂O (10 mL) were added K₂CO₃ (12.01 g, 86.90 mmol) and Pd(dppf)Cl₂CH₂Cl₂ (2.36 g, 2.90 mmol) in portions at rt under nitrogen atmosphere. The resulting mixture was stirred for 4 h at 80° C. under nitrogen atmosphere. On completion, the reaction mixture was cooled to rt and concentrated under vacuum. The residue was purified by silica gel column chromatography, eluted with PE/EA (8:1˜5:1), to afford the title compound (6.00 g, 59% yield) as a yellow solid. LC/MS (ESI, m/z): [(M+H)]⁺=355.2.

### Step 2—Methyl 6-(2,3-dihydro-1H-indol-4-yl)pyridine-3-carboxylate

To a stirred mixture of tert-butyl 4-[5-(methoxycarbonyl)pyridin-2-yl]-2,3-dihydroindole-1-carboxylate (6 g, 20 mmol) in DCM (20 mL) was added TFA (20 mL) dropwise at rt. The resulting solution was stirred for 1.5 h at rt under nitrogen atmosphere. On completion, the resulting solution was concentrated under vacuum. The residue was purified by trituration with Et₂O to give the title compound (3.8 g, 88% yield) as a yellow solid. LC/MS (ESI, m/z): [(M+H)]⁺=255.2.

### Step 3—Methyl 6-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]pyridine-3-carboxylate

To a stirred mixture of methyl 6-(2,3-dihydro-1H-indol-4-yl)pyridine-3-carboxylate (2.00 g, 7.87 mmol) and 6-chloro-N-[(1R,2S)-2-fluorocyclopropyl]-8-{[(4-methoxyphenyl)methyl](methyl)amino}imidazo[1,2-b]pyridazine-3-carboxamide (3.81 g, 9.44 mmol, Intermediate G) in dioxane (20 mL) were added RuPhos-PdOMs-2nd G (0.66 g, 0.79 mmol), RuPhos (0.73 g, 1.6 mmol) and K₂CO₃ (3.26 g, 23.4 mmol) in portions at rt under nitrogen atmosphere. The resulting mixture was stirred for 2 h at 100° C. under nitrogen atmosphere. On completion, the mixture was cooled to rt and concentrated under vacuum. The residue was purified by silica gel column chromatography, eluted with PE/EA (1:1˜9:1), to afford the title compound (3.80 g, 78% yield) as a brown solid. LC/MS (ESI, m/z): [(M+H)]⁺=622.3.

### Step 4—6-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]pyridine-3-carboxylic Acid

To a stirred solution of methyl 6-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]pyridine-3-carboxylate (3.80 g, 6.11 mmol) in THF (30 mL) and H₂O (6 mL) was added LiOH (0.73 g, 31 mmol) at rt under nitrogen atmosphere. The resulting solution was stirred for 2 h at rt under nitrogen atmosphere. On completion, the solution residue was acidified to pH 5 with formic acid and was extracted with EtOAc (5×25 mL). The combined organic layers were washed with EtOAc (2×10 mL), and dried over anhydrous Na₂SO₄. After filtration, the filtrate was concentrated under reduced pressure to give the title compound (3.8 g) as a yellow solid. LC/MS (ESI, m/z): [(M+H)]⁺=608.3.

### Step 5—6-[1-(3-{[(1R,2S)-2-fluorocyclopropyl]carbamoyl}-8-(methylamino)imidazo[1,2-b]pyridazin-6-yl)-2,3-dihydroindol-4-yl]pyridine-3-carboxylic Acid

To a stirred mixture of 6-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]pyridine-3-carboxylic acid (3.80 g, 6.254 mmol) in DCM (20 mL) was added TFA (20 mL) dropwise at rt. The resulting mixture was stirred for 2 h at rt under nitrogen atmosphere. On completion, the resulting mixture was concentrated under vacuum. The residue was purified by trituration with diethyl ether (30 mL) to afford the title compound (2.20 g, 72% yield) as an off-white solid. LC/MS (ESI, m/z): [(M+H)]⁺=488.3.

### Example 1.264. 3-(3-Methyl-2-oxo-5-{4-[4-(piperidin-4-ylmethyl)piperazin-1-yl]phenyl}-1,3-benzodiazol-1-yl)piperidine-2,6-dione (Intermediate JR)

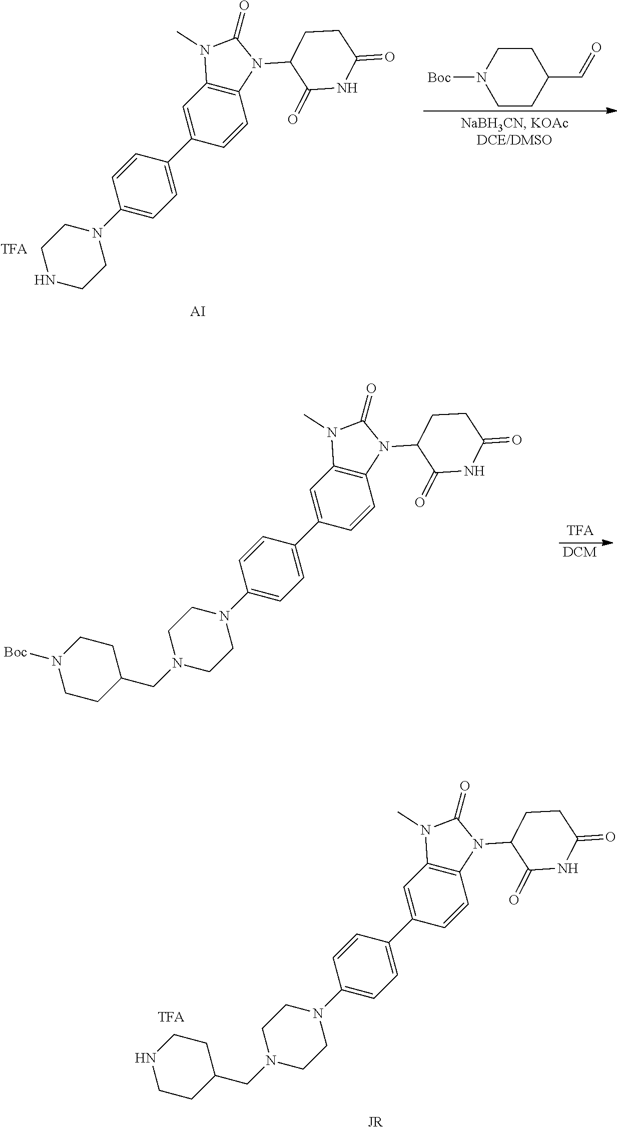

### Step 1—4-[(4-{4-[1-(2,6-Dioxopiperidin-3-yl)-3-methyl-2-oxo-1,3-benzodiazol-5-yl]phenyl}piperazin-1-yl)methyl]piperidine-1-carboxylate

To a stirred solution of 3-{3-methyl-2-oxo-5-[4-(piperazin-1-yl)phenyl]-1,3-benzodiazol-1-yl}piperidine-2,6-dione trifluoroacetate (700 mg, 1 mmol, Intermediate AI) and KOAc (398 mg, 4.06 mmol) in DCE (4 mL) and DMSO (4 mL) was added tert-butyl 4-formylpiperidine-1-carboxylate (288 mg, 1.35 mmol) at rt under nitrogen atmosphere. The resulting mixture was stirred for 30 min at rt under nitrogen atmosphere. To the above mixture was added NaBH₃CN (170 mg, 2.705 mmol) at rt. The resulting mixture was stirred for additional 2 h at rt. On completion, the mixture was concentrated under reduced pressure. The residue was purified by reverse flash chromatography (Column, C18 silica gel; mobile phase A: water (5 mmol/L NH₄HCO₃), mobile phase B: ACN, 50% to 70% gradient in 30 min; detector, UV 254 nm; the fractions were collected at 61%) and concentrated under reduced pressure to afford the title compound (800 mg, 90% yield) as a yellow solid. LC/MS (ESI, m/z): [(M+H)]⁺=617.2.

### Step 2—3-(3-Methyl-2-oxo-5-{4-[4-(piperidin-4-ylmethyl)piperazin-1-yl]phenyl}-1,3-benzodiazol-1-yl)piperidine-2,6-dione Trifluoroacetate

A solution of tert-butyl 4-[(4-{4-[1-(2,6-dioxopiperidin-3-yl)-3-methyl-2-oxo-1,3-benzodiazol-5-yl]phenyl}piperazin-1-yl)methyl]piperidine-1-carboxylate (800 mg, 1 mmol) in TFA (2 mL) and H₂O (6 mL) was stirred for 1 h at rt under nitrogen atmosphere. On completion, the mixture was concentrated under reduced pressure. The residue was purified by trituration with Et₂O (10 mL) to afford the title compound (780 mg) as a yellow solid. LC/MS (ESI, m/z): [(M+H)]⁺=517.2.

### Example 1.265. Preparation of 3-{5-[4-(3,3-difluoropiperidin-4-yl)piperazin-1-yl]-3-methyl-2-oxo-1,3-benzodiazol-1-yl}piperidine-2,6-dione (Intermediate JS)

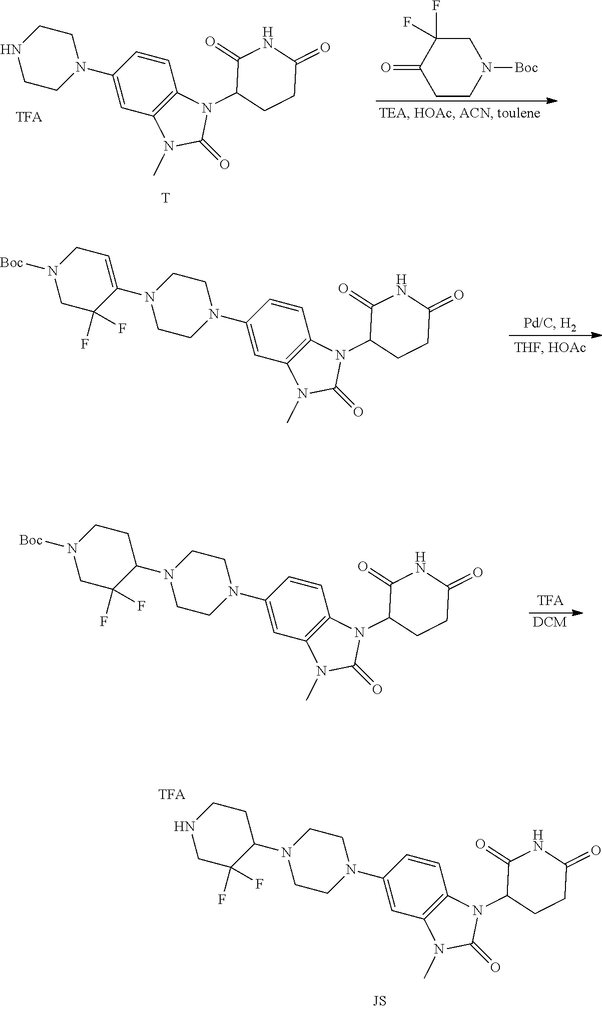

### Step 1—Tert-butyl 4-{4-[1-(2,6-dioxopiperidin-3-yl)-3-methyl-2-oxo-1,3-benzodiazol-5-yl]piperazin-1-yl}-3,3-difluoro-2,6-dihydropyridine-1-carboxylate

To a stirred solution of 3-[3-methyl-2-oxo-5-(piperazin-1-yl)-1,3-benzodiazol-1-yl]piperidine-2,6-dione trifluoroacetate (2.7 g, 6.1 mmol, Intermediate T) in toluene (20 mL) and ACN (20 mL) was added TEA (1.28 mL, 9.18 mmol) dropwise at rt under nitrogen atmosphere. To the above mixture was added tert-butyl 3,3-difluoro-4-oxopiperidine-1-carboxylate (5.76 g, 24.5 mmol, CAS #1215071-17-2) and AcOH (1.10 g, 18.4 mmol) at rt. The resulting mixture was stirred for additional 30 min at rt. Then the mixture was stirred for 16 h at 110° C. under nitrogen atmosphere. On completion, the mixture was cooled to rt and basified to pH 8 with saturated NaHCO₃ (aq.). The resulting mixture was extracted with EtOAc (3×100 mL). The combined organic layers were washed with brine (300 mL), and dried over anhydrous Na₂SO₄. After filtration, the filtrate was concentrated under reduced pressure to afford the title compound (3 g) as a colorless oil. LC/MS (ESI, m/z): [(M+H)]⁺=561.4.

### Step 2—Tert-butyl 4-{4-[1-(2,6-dioxopiperidin-3-yl)-3-methyl-2-oxo-1,3-benzodiazol-5-yl]piperazin-1-yl}-3,3-difluoropiperidine-1-carboxylate

To a solution of tert-butyl 4-{4-[1-(2,6-dioxopiperidin-3-yl)-3-methyl-2-oxo-1,3-benzodiazol-5-yl]piperazin-1-yl}-3,3-difluoro-2,6-dihydropyridine-1-carboxylate (3 g, 5 mmol) in THF (30 mL) was added Pd/C (4.29 g, 74.8 mmol) under nitrogen atmosphere. The reaction system was degassed under vacuum and purged with H₂ several times, then it was hydrogenated under H₂ balloon (˜1 atm) at 25° C. for 3 h. After completion of the reaction, Pd/C was filtered off through celite. The filter cake was washed with THF (3×30 mL) and the filtrate was concentrated. The residue was purified by silica gel column chromatography, eluted with CHCl₃/MeOH (10:1) to afford the title compound (850 mg, 28% yield) as a white solid. LC/MS (ESI, m/z): [(M+H)]⁺=563.4.

### Step 3—3-{5-[4-(3,3-Difluoropiperidin-4-yl)piperazin-1-yl]-3-methyl-2-oxo-1,3-benzodiazol-1-yl}piperidine-2,6-dione Trifluoroacetate

To a stirred solution of tert-butyl 4-{4-[1-(2,6-dioxopiperidin-3-yl)-3-methyl-2-oxo-1,3-benzodiazol-5-yl]piperazin-1-yl}-3,3-difluoropiperidine-1-carboxylate (850 mg, 1.5 mmol) in DCM (10 mL) was added TFA (4 mL) at 0° C. under nitrogen atmosphere. The resulting mixture was stirred for 2 h at rt under nitrogen atmosphere. On completion, the mixture was concentrated under reduced pressure. The residue was purified by trituration with Et₂O (60 mL) to afford the title compound (800 mg, 95% yield) as an off-white solid. LC/MS (ESI, m/z): [(M+H)]⁺=563.3.

### Example 1.266. 3-[3-Methyl-2-oxo-5-(4-oxocyclohexyl)-1,3-benzodiazol-1-yl]piperidine-2,6-dione (Intermediate JT)

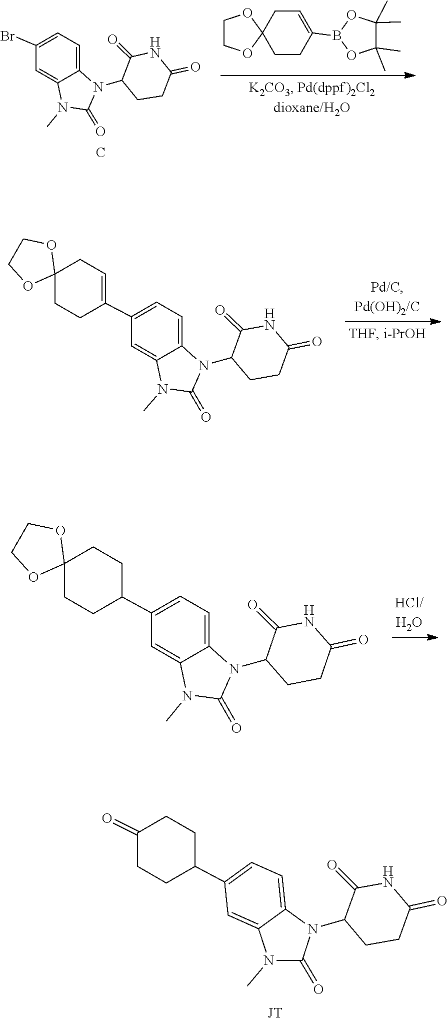

### Step 1—3-(5-{1,4-Dioxaspiro[4.5]dec-7-en-8-yl}-3-methyl-2-oxo-1,3-benzodiazol-1-yl)piperidine-2,6-dione

To a stirred solution of 3-(5-bromo-3-methyl-2-oxo-1,3-benzodiazol-1-yl)piperidine-2,6-dione (5 g, 10 mmol, Intermediate C) and 2-{1,4-dioxaspiro[4.5]dec-7-en-8-yl}-4,4,5,5-tetramethyl-1,3,2-dioxaborolane (4.72 g, 17.7 mmol, CAS #680596-79-6) in dioxane (50 mL) and H₂O (10 mL) were added Pd(dppf)Cl₂·CH₂Cl₂ (1.20 g, 1.48 mmol) and K₂CO₃ (4.09 g, 29.6 mmol) in portions at rt under nitrogen atmosphere. The resulting mixture was stirred for 1 h at 90° C. under nitrogen atmosphere. On completion, the mixture was concentrated under reduced pressure. The residue was purified by silica gel column chromatography, eluted with CH₂Cl₂/MeOH (10:1), to afford the title compound (4.5 g, 77% yield) as a brown yellow solid. LC/MS (ESI, m/z): [(M+H)]⁺=398.2.

### Step 2—3-(5-{1,4-Dioxaspiro[4.5]decan-8-yl}-3-methyl-2-oxo-1,3-benzodiazol-1-yl)piperidine-2,6-dione

To a solution of 3-(5-{1,4-dioxaspiro[4.5]dec-7-en-8-yl}-3-methyl-2-oxo-1,3-benzodiazol-1-yl)piperidine-2,6-dione (4.5 g, 11 mmol) in THF (5 mL) and i-PrOH (50 mL) were added Pd/C (10 wt %, 450 mg) and Pd(OH)₂/C (450 mg, 0.036 mmol, 10 wt %) under nitrogen atmosphere. The mixture was hydrogenated at rt for 16 h under hydrogen atmosphere using a hydrogen balloon. On completion, the mixture was filtered and the filter cake was washed with THF (6×20 mL). The filtrate was concentrated under reduced pressure to afford the title compound (3.8 g, 84% yield) as a yellow green solid. LC/MS (ESI, m/z): [(M+H)]⁺=400.2.

### Step 3—3-[3-Methyl-2-oxo-5-(4-oxocyclohexyl)-1,3-benzodiazol-1-yl]piperidine-2,6-dione

To a stirred solution of 3-(5-{1,4-dioxaspiro[4.5]decan-8-yl}-3-methyl-2-oxo-1,3-benzodiazol-1-yl)piperidine-2,6-dione (3.8 g, 9.513 mmol) in H₂O (20 mL) was added 1 M aq. HCl (20 mL) dropwise at rt under nitrogen atmosphere. The resulting mixture was stirred for 16 h at 60° C. under nitrogen atmosphere. On completion, the mixture was cooled to rt and the precipitated solids were collected by filtration and washed with H₂O (3×10 mL). The collected solid was dried under reduced pressure to afford the title compound (2.7 g, 80% yield) as a light grey solid. LC/MS (ESI, m/z): [(M+H)]⁺=356.2.

### Example 1.267. Preparation of Tert-butyl 4-[(1r,4r)-4-(1-(2,6-dioxopiperidin-3-yl)-3-methyl-2-oxo-1,3-benzodiazol-5-yl]cyclohexyl]piperazine-1-carboxylate (Intermediate JU) and tert-butyl 4-[(1s,4s)-4-[1-(2,6-dioxopiperidin-3-yl)-3-methyl-2-oxo-1,3-benzodiazol-5-yl]cyclohexyl]piperazine-1-carboxylate (Intermediate JV)

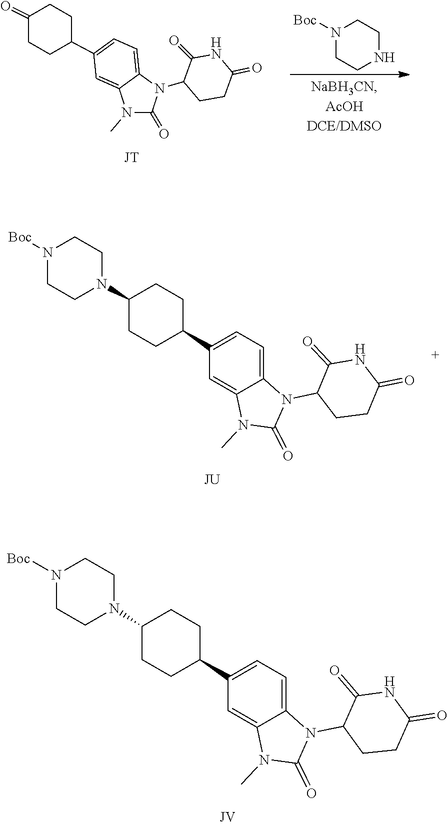 [SMILES: Cn1c(=O)n(C2CCC(=O)NC2=O)c2ccc(C3CCC(=O)CC3)cc21 ; CC(C)(C)OC(=O)N1CCNCC1 ; Cn1c(=O)n(C2CCC(=O)NC2=O)c2ccc([C@H]3CC[C@@H](N4CCN(C(=O)OC(C)(C)C)CC4)CC3)cc21 ; Cn1c(=O)n(C2CCC(=O)NC2=O)c2ccc([C@H]3CC[C@H](N4CCN(C(=O)OC(C)(C)C)CC4)CC3)cc21]

To a stirred solution of 3-[3-methyl-2-oxo-5-(4-oxocyclohexyl)-1,3-benzodiazol-1-yl]piperidine-2,6-dione (2.7 g, 7.6 mmol, Intermediate JT) and tert-butyl piperazine-1-carboxylate (1.70 g, 9.12 mmol) in DCE (20 mL) and DMSO (20 mL) were added HOAc (0.1 mL, 1.7 mmol) at rt under nitrogen atmosphere. The resulting mixture was stirred for 2 h at rt under nitrogen atmosphere. To the above mixture was added NaBH₃CN (954.82 mg, 15.194 mmol) at 0° C. The resulting mixture was stirred for additional 16 h at rt. On completion, the mixture was concentrated under reduced pressure. The residue was purified by reverse phase Flash chromatography (Column: WelFlash™ C18-I, 20-40 um, 330 g; Eluent A: Water (plus 10 mmol/L NH₄HCO₃); Eluent B: ACN; Gradient: 25%-55% B in 25 min; Flow rate: 80 mL/min; Detector: 254 nm). Desired fractions were collected at 35% B and concentrated under reduced pressure to afford tert-butyl 4-[(1r,4r)-4-[1-(2,6-dioxopiperidin-3-yl)-3-methyl-2-oxo-1,3-benzodiazol-5-yl]cyclohexyl]piperazine-1-carboxylate (600 mg, 15% yield) as an off-white solid LC/MS (ESI, m/z): [(M+H)]⁺=526.3. Desired fractions were also collected at 45% B and concentrated under reduced pressure to afford tert-butyl 4-[(1s,4s)-4-[1-(2,6-dioxopiperidin-3-yl)-3-methyl-2-oxo-1,3-benzodiazol-5-yl]cyclohexyl]piperazine-1-carboxylate (600 mg, 15% yield) as an off-white solid. LC/MS (ESI, m/z): [(M+H)]⁺=526.3. The cis/trans isomers were confirmed by NOESY, and absolute stereochemistry was assigned arbitrarily.

### Example 1.268. Methyl 6-(3-amino-2-methoxyphenyl)pyridine-3-carboxylate (Intermediate JW)

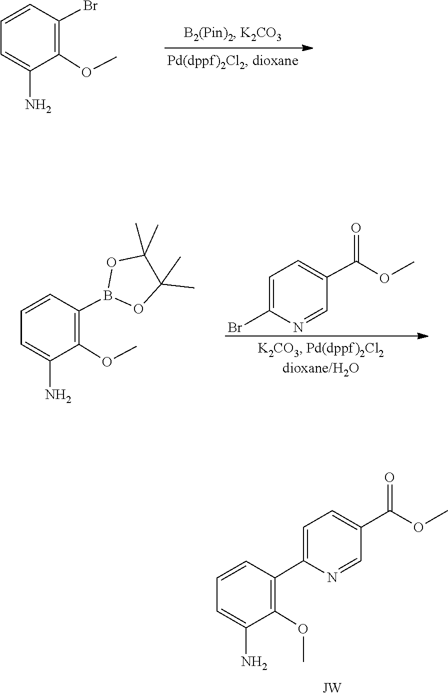

### Step 1—2-Methoxy-3-(4,4,5,5-tetramethyl-1,3,2-dioxaborolan-2-yl)aniline

To a stirred solution of 3-bromo-2-methoxyaniline (10.00 g, 49.49 mmol) and bis(pinacolato)diboron (18.85 g, 74.24 mmol) in dioxane (100 mL) was added KOAc (14.57 g, 148.5 mmol) and Pd(dppf)Cl₂ (3.62 g, 4.95 mmol) at rt under nitrogen atmosphere. The resulting mixture was stirred for 2 h at 100° C. under nitrogen atmosphere. On completion, the reaction mixture was cooled to rt and concentrated under reduced pressure. The residue was purified by silica gel column chromatography, eluted with PE/EA (3:1) to afford the title compound (9.3 g, 75% yield) as a yellow solid. LC/MS (ESI, m/z): [(M+H)]⁺=250.3.

### Step 2—Methyl 6-(3-amino-2-methoxyphenyl)pyridine-3-carboxylate

To a stirred solution of 2-methoxy-3-(4,4,5,5-tetramethyl-1,3,2-dioxaborolan-2-yl)aniline (9.20 g, 36.9 mmol) and methyl 6-bromopyridine-3-carboxylate (7.98 g, 36.9 mmol) in dioxane (80 mL) and H₂O (20 mL) was added K₂CO₃ (10.21 g, 73.86 mmol) and Pd(dppf)Cl₂ (5.40 g, 7.39 mmol) at rt under nitrogen atmosphere. The resulting mixture was stirred for 2 h at 90° C. under nitrogen atmosphere. On completion, the reaction mixture was cooled to rt and concentrated under reduced pressure. The residue was purified by silica gel column chromatography, eluted with PE/EA (1:1), to afford the title compound (10.04 g, quant. yield) as a yellow solid. LC/MS (ESI, m/z): [(M+H)]⁺=259.2.

### Example 1.269. Preparation of 5-(1,3-dioxolan-2-yl)-2-(tributylstannyl)pyridine (Intermediate JX)

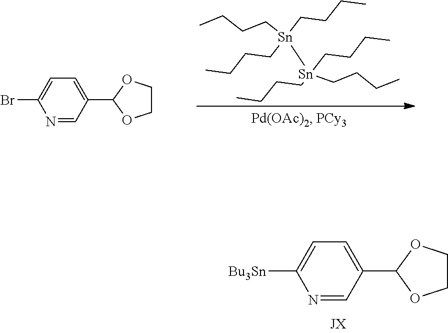

To a mixture of 2-bromo-5-(1,3-dioxolan-2-yl)pyridine (6.7 g, 29 mmol, CAS #220904-17-6) and Pd(OAc)2 (0.07 g, 0.3 mmol) in hexabutyldistannane (18.58 g, 32.04 mmol) was added PCy₃ (0.16 g, 0.58 mmol) in portions at rt under nitrogen atmosphere. The resulting mixture was stirred for 2 h at 110° C. under nitrogen atmosphere. On completion, the mixture was cooled to rt and concentrated under reduced pressure. The residue was purified by silica gel column chromatography, eluted with PE/EA (3:1) to afford the title compound (5.7 g, 45% yield) as a yellow oil. LC/MS (ESI, m/z): [(M+H)]⁺=442.3.

### Example 1.270. Preparation of 6-({3-[5-(1,3-dioxolan-2-yl)pyridin-2-yl]-2-methoxyphenyl}amino)-8-{[(4-methoxyphenyl)methyl](methyl)amino}imidazo[1,2-b]pyridazine-3-carboxylic Acid (Intermediate JY)

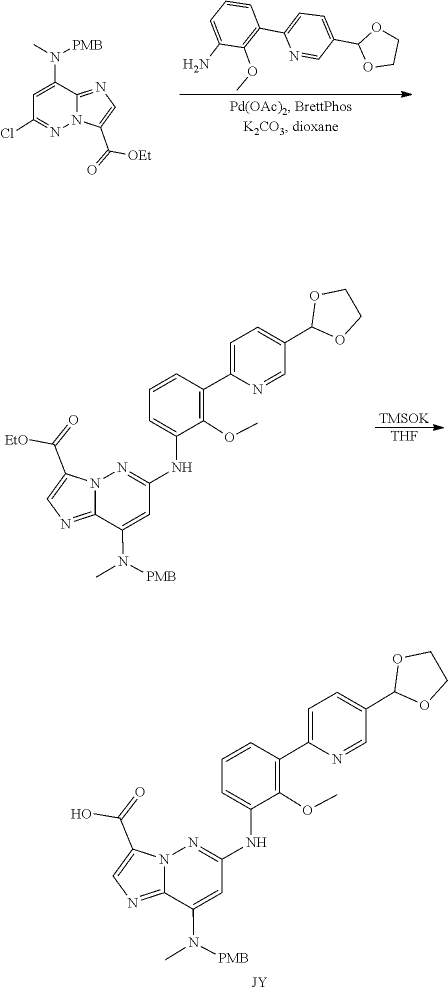

### Step 1—Ethyl 6-({3-[5-(1,3-dioxolan-2-yl)pyridin-2-yl]-2-methoxyphenyl}amino)-8-{[(4-methoxyphenyl)methyl](metEhyl)amino}imidazo[1,2-b]pyridazine-3-carboxylate

To a stirred solution of ethyl 6-chloro-8-{[(4-methoxyphenyl)methyl](methyl)amino}imidazo[1,2-b]pyridazine-3-carboxylate (5 g, 10 mmol, synthesized via Steps 1-2 of Intermediate F) and 3-[5-(1,3-dioxolan-2-yl)pyridin-2-yl]-2-methoxyaniline (4.36 g, 16.0 mmol, synthesized via Steps 1-4 of Intermediate IS) in 1,4-dioxane (100 mL) were added Pd(OAc)₂ (0.30 g, 1.33 mmol), BrettPhos (0.72 g, 1.33 mmol) and K₂CO₃ (3.69 g, 26.7 mmol) in portions at rt under nitrogen atmosphere. The resulting mixture was stirred for 16 h at 80° C. under nitrogen atmosphere. On completion, the mixture was cooled to rt and concentrated under vacuum. The resulting mixture was diluted with water (100 mL) and extracted with EtOEt (2×200 mL). The combined organic layers were washed with water (100 mL), and dried over anhydrous Na₂SO₄. After filtration, the filtrate was concentrated under reduced pressure. The residue was purified by silica gel column chromatography, eluted with PE/EA (1:1), to afford the title compound (7.0 g, 86% yield) as an off-white solid. LC/MS (ESI, m/z): [(M+H)]⁺=611.4.

### Step 2—6-({3-[5-(1,3-Dioxolan-2-yl)pyridin-2-yl]-2-methoxyphenyl}amino)-8-{[(4-methoxyphenyl)methyl](methyl)amino}imidazo[1,2-b]pyridazine-3-carboxylic Acid

To a stirred solution of ethyl 6-({3-[5-(1,3-dioxolan-2-yl)pyridin-2-yl]-2-methoxyphenyl}amino)-8-{[(4-methoxyphenyl)methyl](methyl)amino}imidazo[1,2-b]pyridazine-3-carboxylate (7 g, 11.463 mmol) in THF (30 mL) was added potassium trimethylsilanolate (2.93 g, 22.9 mmol) at rt. The resulting mixture was stirred for 2 h at rt under nitrogen atmosphere. On completion, the mixture was concentrated under reduced pressure. The residue was purified by trituration with EtOAc (30 mL) to give the title compound as the potassium salt (6.0 g, 84% yield) as a brown solid. LC/MS (ESI, m/z): [(M+H)]⁺=583.3.

### Example 1.271. Preparation of N-[(1R,2S)-2-fluorocyclopropyl]-6-[4-(4-fluoropyridin-2-yl)-2,3-dihydroindol-1-yl]-8-{[(4-methoxyphenyl)methyl](methyl)amino}imidazo[1,2-b]pyridazine-3-carboxamide (Intermediate JZ)

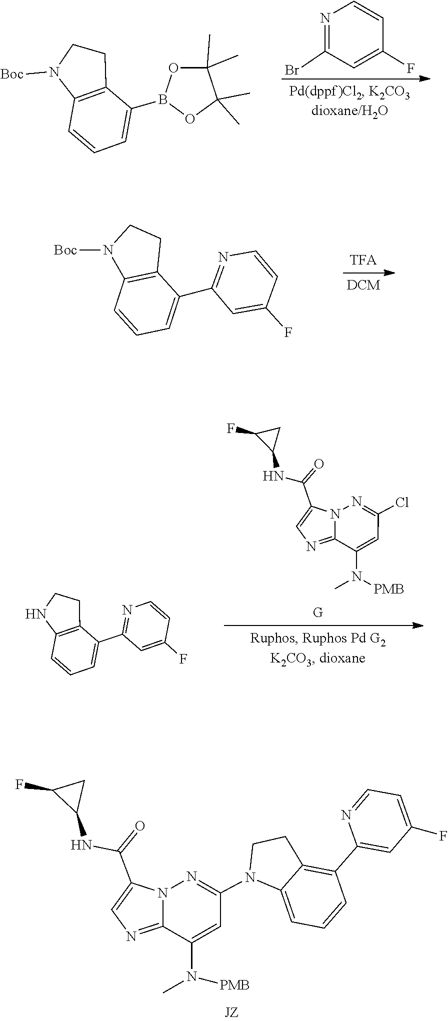

### Step 1—Tert-butyl 4-(4-fluoropyridin-2-yl)-2,3-dihydroindole-1-carboxylate

To a stirred mixture of tert-butyl 4-(4,4,5,5-tetramethyl-1,3,2-dioxaborolan-2-yl)-2,3-dihydroindole-1-carboxylate (5.5 g, 16 mmol, synthesized via Step 1 of Intermediate IN), 2-bromo-4-fluoropyridine (3.08 g, 17.5 mmol) and Na₂CO₃ (5.07 g, 47.8 mmol) in dioxane (500 mL) and H₂O (50 mL) was added Pd(dppf)Cl₂·CH₂Cl₂ (1.30 g, 1.59 mmol) at rt under nitrogen atmosphere. The resulting mixture was stirred for 16 h at 80° C. under nitrogen atmosphere. On completion, the mixture was cooled to rt and concentrated under reduced pressure. The residue was purified by silica gel column chromatography, eluted with PE/EA (10:1), to afford the title compound (3.4 g, 68% yield) as a brown yellow solid. LC/MS (ESI, m/z): [(M+H)]⁺=315.2.

### Step 2—4-(4-Fluoropyridin-2-yl)-2,3-dihydro-1H-indole

To a stirred solution of tert-butyl 4-(4-fluoropyridin-2-yl)-2,3-dihydroindole-1-carboxylate (2 g, 6 mmol) in DCM (10 mL) was added TFA (2 mL) dropwise at rt. The resulting mixture was stirred for 2 h at rt. On completion, the mixture was concentrated under reduced pressure. The residue was triturated with Et₂O to afford the title compound (1.3 g, 95% yield) as a light yellow solid. LC/MS (ESI, m/z): [(M+H)]⁺=215.2.

### Step 3—N-[(1R,2S)-2-fluorocyclopropyl]-6-[4-(4-fluoropyridin-2-yl)-2,3-dihydroindol-1-yl]-8-{[(4-methoxyphenyl)methyl](methyl)amino}imidazo[1,2-b]pyridazine-3-carboxamide

To a stirred mixture of 4-(4-fluoropyridin-2-yl)-2,3-dihydro-1H-indole (1.3 g, 6.068 mmol), 6-chloro-N-[(1R,2S)-2-fluorocyclopropyl]-8-{[(4-methoxyphenyl)methyl](methyl)amino}imidazo[1,2-b]pyridazine-3-carboxamide (2.70 g, 6.68 mmol, Intermediate G) and K₂CO₃ (2.52 g, 18.2 mmol) in dioxane (20 mL) were added RuPhos (0.28 g, 0.61 mmol) and RuPhos-PdCl-2nd G (0.47 g, 0.61 mmol) at rt under nitrogen atmosphere. The resulting mixture was stirred for 16 h at 80° C. under nitrogen atmosphere. On completion, the mixture was cooled to rt and concentrated under reduced pressure. The residue was purified by silica gel column chromatography, eluted with PE/EA (1/1), to afford the title compound (900 mg, 26% yield) as a brown yellow solid. LC/MS (ESI, m/z): [(M+H)]⁺=582.3.

### Example 1.272. Preparation of 3-{3-methyl-2-oxo-4-[4-(piperazin-1-yl)phenyl]-1,3-benzodiazol-1-yl}piperidine-2,6-dione (Intermediate KA)

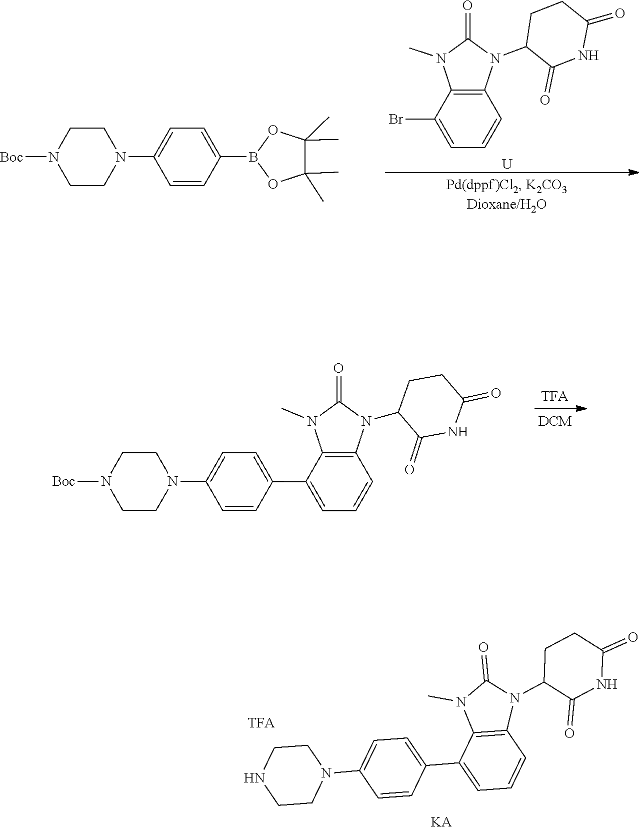

### Step 1—Tert-butyl 4-{4-[1-(2,6-dioxopiperidin-3-yl)-3-methyl-2-oxo-1,3-benzodiazol-4-yl]phenyl}piperazine-1-carboxylate

To a stirred solution of tert-butyl 4-[4-(4,4,5,5-tetramethyl-1,3,2-dioxaborolan-2-yl)phenyl]piperazine-1-carboxylate (1 g, 3 mmol, CAS #470478-90-1) and 3-(4-bromo-3-methyl-2-oxo-1,3-benzodiazol-1-yl)piperidine-2,6-dione (0.87 g, 2.6 mmol, Intermediate U) in dioxane (20 mL) and H₂O (4 mL) were added K₂CO₃ (0.71 g, 5.2 mmol) and Pd(dppf)Cl₂CH₂Cl₂ (0.21 g, 0.26 mmol) at rt under nitrogen atmosphere. The resulting mixture was stirred for 2 h at 80° C. under nitrogen atmosphere. On completion, the mixture was cooled to rt and concentrated under reduced pressure. The residue was purified by reverse flash chromatography (column, C18 silica gel; mobile phase, ACN in Water (0.1% FA), 40% to 60% gradient in 20 min; detector, UV 254 nm; desired fraction were collected at 51% B) to afford the title compound (640 mg, 48% yield) as a brown solid. LC/MS (ESI, m/z): [(M+1)]⁺=520.3.

### Step 2—3-{3-Methyl-2-oxo-4-[4-(piperazin-1-yl)phenyl]-1,3-benzodiazol-1-yl}piperidine-2,6-dione trifluoroacetate

To a stirred solution of tert-butyl 4-{4-[1-(2,6-dioxopiperidin-3-yl)-3-methyl-2-oxo-1,3-benzodiazol-4-yl]phenyl}piperazine-1-carboxylate (640 mg, 1.23 mmol) in DCM (10 mL) was added TFA (2 mL) at rt under nitrogen atmosphere. The resulting mixture was stirred for 4 h at rt under nitrogen atmosphere. On completion, the mixture was concentrated under reduced pressure. The residue was purified by trituration with diethyl ether to give the title compound (900 mg) as a brown solid. LC/MS (ESI, m/z): [(M+1)]⁺=420.2.

### Example 1.273, 3-{3-methyl-2-oxo-4-[2-(piperidin-4-yl)ethyl]-1,3-benzodiazol-1-yl}piperidine-2,6-dione (Intermediate KB)

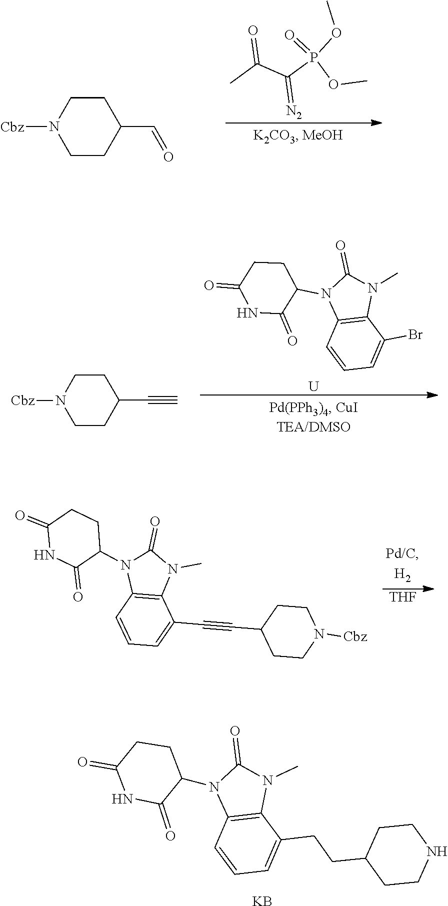

### Step 1—Benzyl 4-ethynylpiperidine-1-carboxylate

To a stirred solution of benzyl 4-formylpiperidine-1-carboxylate (10 g, 40 mmol, CAS #138163-08-3) and seyferth-gilbert homologation (7.77 g, 40.4 mmol, CAS #90965-06-3) in MeOH (100 mL) was added K₂CO₃ (11.18 g, 80.88 mmol) at rt under nitrogen atmosphere. The resulting mixture was stirred for 2 h at rt under nitrogen atmosphere. On completion, the mixture was concentrated under reduced pressure. The residue was purified by silica gel column chromatography, eluted with PE/EA (10:1), to afford the title compound (8 g, 81% yield) as a white solid. LC/MS (ESI, m/z): [(M+H)]⁺=244.1.

### Step 2—Benzyl 4-{2-[1-(2,6-dioxopiperidin-3-yl)-3-methyl-2-oxo-1,3-benzodiazol-4-yl]ethynyl}piperidine-1-carboxylate

To a stirred solution of benzyl 4-ethynylpiperidine-1-carboxylate (8 g, 30 mmol) and 3-(4-bromo-3-methyl-2-oxo-1,3-benzodiazol-1-yl)piperidine-2,6-dione (11.12 g, 32.88 mmol, Intermediate U) in TEA (200 mL) and DMSO (200 mL) were added Pd(PPh₃)₄ (3.80 g, 3.29 mmol) and CuI (0.33 g, 3.3 mmol) in portions at rt under nitrogen atmosphere. The resulting mixture was stirred for 2 h at 80° C. under nitrogen atmosphere. On completion, the mixture was cooled to rt and acidified to pH 5 with conc. HCl. The aqueous layer was extracted with EtOAc (100 mL). The combined organic layer was concentrated under reduced pressure. The residue was purified by silica gel column chromatography, eluted with CH₂Cl₂/EA (2:3) and CH₂Cl₂/MeOH (10:1), to afford the title compound (8 g, 49% yield) as a brown solid. LC/MS (ESI, m/z): [(M+H)]⁺=501.3.

### Step 3—3-{3-Methyl-2-oxo-4-[2-(piperidin-4-yl)ethyl]-1,3-benzodiazol-1-yl}piperidine-2,6-dione

To a solution of benzyl 4-{2-[1-(2,6-dioxopiperidin-3-yl)-3-methyl-2-oxo-1,3-benzodiazol-4-yl]ethynyl}piperidine-1-carboxylate (1.0 g, 2.0 mmol) in THF (20 mL) was added Pd/C (0.5 g, 10 wt %) under nitrogen atmosphere. The mixture was hydrogenated at rt for 4 h under hydrogen atmosphere using a hydrogen balloon. On completion, the mixture was filtered through a celite pad and concentrated under reduced pressure. The resulting mixture was filtered, the filter cake was washed with MeOH (10 mL). The filtrate was then concentrated under reduced pressure to afford the title compound (250 mg, 34% yield) as a white solid. LC/MS (ESI, m/z): [(M+H)]⁺=371.2.

### Example 1.274. Preparation of 1-{5-[4-(azetidin-3-yl)piperidine-1-carbonyl]-2-chlorophenyl}-1,3-diazinane-2,4-dione (Intermediate KC)

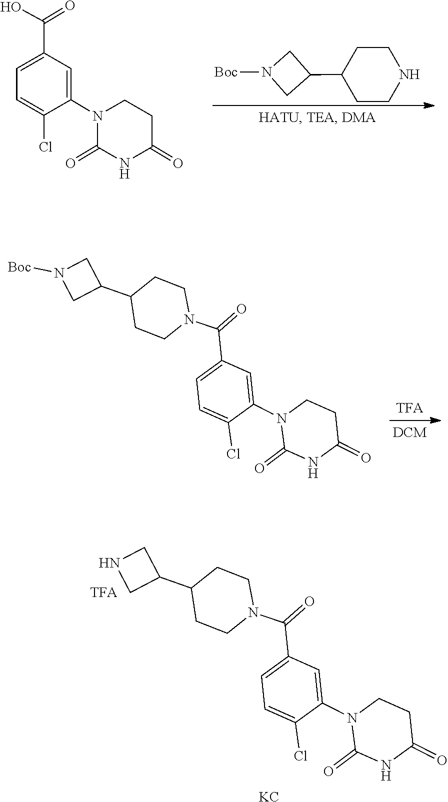 [SMILES: O=C1CCN(c2cc(C(=O)O)ccc2Cl)C(=O)N1 ; CC(C)(C)OC(=O)N1CC(C2CCNCC2)C1 ; CC(C)(C)OC(=O)N1CC(C2CCN(C(=O)c3ccc(Cl)c(N4CCC(=O)NC4=O)c3)CC2)C1 ; O=C1CCN(c2cc(C(=O)N3CCC(C4CNC4)CC3)ccc2Cl)C(=O)N1 ; "KC"]

### Step 1—Tert-butyl 3-{1-[4-chloro-3-(2,4-dioxo-1,3-diazinan-1-yl)benzoyl]piperidin-4-yl}azetidine-1-carboxylate

To a stirred solution of 4-chloro-3-(2,4-dioxo-1,3-diazinan-1-yl)benzoic acid (1.12 g, 4.16 mmol, synthesized via Steps 1-3 of Intermediate IO) and HATU (1.90 g, 4.99 mmol) in DMA (1 mL) were added tert-butyl 3-(piperidin-4-yl)azetidine-1-carboxylate (1 g, 4 mmol, CAS #1251006-64-0) and TEA (1.26 g, 12.483 mmol) at rt under nitrogen atmosphere. The resulting mixture was stirred for 3 h at rt under nitrogen atmosphere. On completion, the mixture was concentrated under reduced pressure. The residue was purified by reverse flash chromatography (column, C18 silica gel; mobile phase, ACN in Water (0.1% FA), 20% to 40% gradient in 20 min; detector, UV 254 nm; desired fraction were collected at 32% B) to afford the title compound (1 g, 49% yield) as a yellow solid. LC/MS (ESI, m/z): [(M+1)]⁺=491.2.

### Step 2—1-{5-[4-(Azetidin-3-yl)piperidine-1-carbonyl]-2-chlorophenyl}-1,3-diazinane-2,4-dione Trifluoroacetate

To a stirred solution of tert-butyl 3-{1-[4-chloro-3-(2,4-dioxo-1,3-diazinan-1-yl)benzoyl]piperidin-4-yl}azetidine-1-carboxylate (1 g, 2 mmol) in DCM (15 mL) was added TFA (5 mL) at rt under nitrogen atmosphere. The resulting mixture was stirred for 2 h at rt under nitrogen atmosphere. On completion, the mixture was cooled to rt and concentrated under reduced pressure. The residue was purified by trituration with diethyl ether to afford the title compound (1 g) as a yellow solid. LC/MS (ESI, m/z): [(M+1)]⁺=390.9.

### Example 1.275. Preparation of N-((1R,2S)-2-fluorocyclopropyl)-6-((3-formylphenyl)amino)-8-(methylamino)imidazo[1,2-b]pyridazine-3-carboxamide (Intermediate KD)

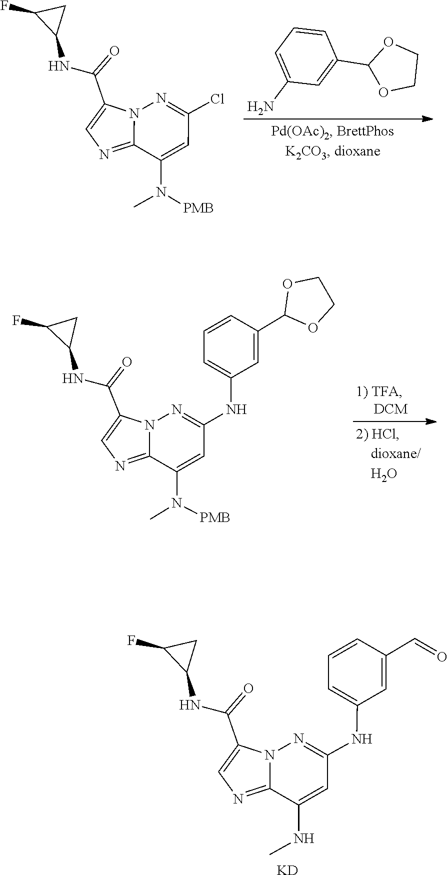

### Step 1—6-{[3-(1,3-Dioxolan-2-yl)phenyl]amino}-N-[(1R,2S)-2-fluorocyclopropyl]-8-{[(4-methoxyphenyl)methyl](methyl)amino}imidazo[1,2-b]pyridazine-3-carboxamide

To a stirred solution of 6-chloro-N-[(1R,2S)-2-fluorocyclopropyl]-8-{[(4-methoxyphenyl)methyl](methyl)amino}imidazo[1,2-b]pyridazine-3-carboxamide (4.89 g, 12.1 mmol, Intermediate G) and 3-(1,3-dioxolan-2-yl)aniline (2 g, 10 mmol, CAS #6398-87-4) in dioxane (50 mL) were added BrettPhos (649.89 mg, 1.211 mmol), Pd(OAc)₂ (271.82 mg, 1.211 mmol) and K₂CO₃ (3.35 g, 24.2 mmol) in portions at rt under nitrogen atmosphere. The resulting mixture was stirred for 16 h at 90° C. under nitrogen atmosphere. On completion, the mixture was cooled to rt and the precipitated solids were collected by filtration. The filter cake was washed with MeOH (3×50 mL) and dried under vacuum to afford the title compound (1.6 g) as a grey solid. LC/MS (ESI, m/z): [(M+H)]⁺=533.1.

### Step 2—N-[(1R,2S)-2-fluorocyclopropyl]-6-[(3-formylphenyl)amino]-8-(methylamino)imidazo[1,2-b]pyridazine-3-carboxamide

To a stirred solution of 6-{[3-(1,3-dioxolan-2-yl)phenyl]amino}-N-[(1R,2S)-2-fluorocyclopropyl]-8-{[(4-methoxyphenyl)methyl](methyl)amino}imidazo[1,2-b]pyridazine-3-carboxamide (100 mg, 0.2 mmol) in DCM (5 mL) was added TFA (1 mL) dropwise at rt. The resulting mixture was stirred for 1 h at rt under nitrogen atmosphere. Once deprotection of PMB group was complete, the resulting mixture was concentrated under reduced pressure. The residue was re-dissolved in dioxane (3 mL). To the above mixture was added HCl (1 mL, 1 mmol) dropwise at rt. The resulting mixture was stirred for additional 6 h at 60° C. On completion, the mixture was concentrated under reduced pressure. The residue was purified by reverse phase Flash chromatography (Column: WelFlash™ C18-I, 20-40 um, 120 g; Eluent A: Water (plus 10 mmol/L NH₄HCO₃); Eluent B: ACN; Gradient: 25%-55% B in 30 min; Flow rate: 60 mL/min; Detector: 254 nm; desired fractions were collected at 42% B) to afford the title compound (14.2 mg, 20% yield) as a yellow solid. LC/MS (ESI, m/z): [(M+H)]⁺=369.1.

### Example 1.276. Preparation of 3-{3-methyl-2-oxo-5-[(1s,4s)-4-(piperazin-1-yl)cyclohexyl]-1,3-benzodiazol-1-yl}piperidine-2,6-dione (Intermediate KE)

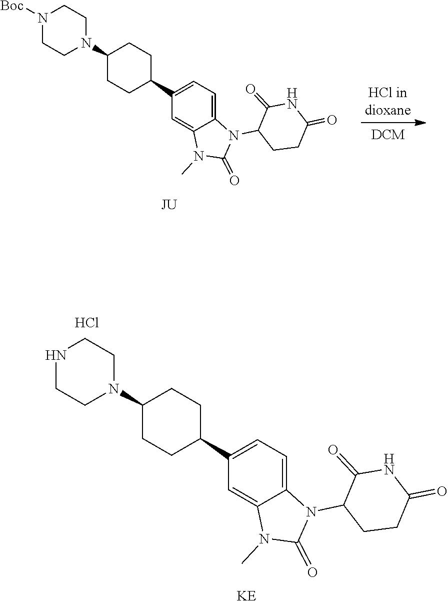 [SMILES: Cn1c(=O)n(C2CCC(=O)NC2=O)c2ccc([C@H]3CC[C@@H](N4CCN(C(=O)OC(C)(C)C)CC4)CC3)cc21 ; Cl ; Cn1c(=O)n(C2CCC(=O)NC2=O)c2ccc([C@H]3CC[C@@H](N4CCNCC4)CC3)cc21]

To a stirred solution of tert-butyl 4-[(1s,4s)-4-[1-(2,6-dioxopiperidin-3-yl)-3-methyl-2-oxo-1,3-benzodiazol-5-yl]cyclohexyl]piperazine-1-carboxylate (600 mg, 1 mmol, Intermediate JU) in DCM (10 mL) was added 4 M HCl (gas) in 1,4-dioxane (2 mL) dropwise at rt under nitrogen atmosphere. The resulting mixture was stirred for 2 h at rt under nitrogen atmosphere. On completion, the mixture was concentrated under reduced pressure. The residue was purified by trituration with ethyl ether (50 mL) to afford the title compound (480 mg, 91% yield) as an off-white solid. LC/MS (ESI, m/z): [(M+H)]⁺=426.3.

### Example 1.277. Preparation of 3-{3-[(3-{[(1R,2S)-2-fluorocyclopropyl]carbamoyl}-8-(methylamino)imidazo[1,2-b]pyridazin-6-yl)amino]-2-methoxyphenyl}prop-2-yn-1-yl methanesulfonate (Intermediate KF)

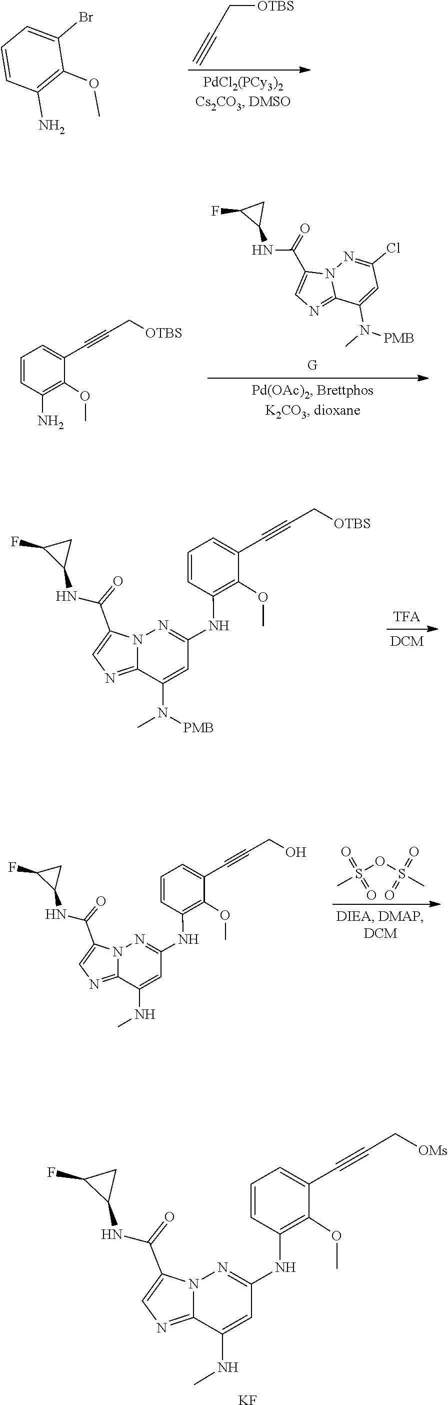

### Step 1—3-{3-[(Tert-butyldimethylsilyl)oxy]prop-1-yn-1-yl}-2-methoxyaniline

To a stirred solution of 3-bromo-2-methoxyaniline (3 g, 10 mmol, CAS #116557-46-1) and tert-butyldimethyl(prop-2-yn-1-yloxy)silane (5.06 g, 30.0 mmol, CAS #76782-82-6) in DMSO (20 mL) was added Cs₂CO₃ (14.51 g, 44.54 mmol) at rt under nitrogen atmosphere. To the above mixture was added dichloropalladium bis(tricyclohexylphosphane) (2.19 g, 2.97 mmol) in portions over 5 min at rt. The resulting mixture was stirred for additional 5 min at rt. Then the mixture was stirred for 2 h at 100° C. under nitrogen atmosphere. On completion, the reaction was cooled to rt and mixture was filtered, then the filter cake was washed with MeCN (20 mL). The filtrate was concentrated under reduced pressure. The residue was purified by reverse phase flash chromatography (Column: WelFlash™ C18-I, 20-40 μm, 330 g; Eluent A: Water (plus 5 mmol/L NH4HCO3); Eluent B: ACN; Gradient: 35%-55% B in 35 min; Flow rate: 80 mL/min; Detector: 220/254 nm; desired fractions were collected at 48% B) to afford the title compound (2 g, 46% yield) as an off-white solid. LC/MS (ESI, m/z): [(M+H)]⁺=292.2.

### Step 2—6-[(3-{3-[(Tert-butyldimethylsilyl)oxy]prop-1-yn-1-yl}-2-methoxyphenyl)amino]-N-[(1R,2S)-2-fluorocyclopropyl]-8-{[(4-methoxyphenyl)methyl](methyl)amino}imidazo[1,2-b]pyridazine-3-carboxamide

To a stirred solution of 3-{3-[(tert-butyldimethylsilyl)oxy]prop-1-yn-1-yl}-2-methoxyaniline (2 g, 7 mmol) and 6-chloro-N-[(1R,2S)-2-fluorocyclopropyl]-8-{[(4-methoxyphenyl)methyl](methyl)amino}imidazo[1,2-b]pyridazine-3-carboxamide (2.77 g, 6.86 mmol, Intermediate G) in dioxane (20 mL) were added K₂CO₃ (1.90 g, 13.7 mmol) and BrettPhos (0.74 g, 1.4 mmol) at rt under nitrogen atmosphere. To the above mixture was added Pd(OAc)₂ (0.15 g, 0.69 mmol) at rt under nitrogen atmosphere. The resulting mixture was stirred for additional 5 min at rt. On completion, the mixture was stirred for 2 h at 100° C. under nitrogen atmosphere. On completion, the mixture was cooled to rt and concentrated under reduced pressure. The residue was purified by silica gel column chromatography, eluted with PE/EA (2:1), to afford the title compound (1 g, 22% yield) as a light brown solid. LC/MS (ESI, m/z): [(M+H)]⁺=659.3.

### Step 3—N-[(1R,2S)-2-fluorocyclopropyl]-6-{[3-(3-hydroxyprop-1-yn-1-yl)-2-methoxyphenyl]amino}-8-(methylamino)imidazo[1,2-b]pyridazine-3-carboxamide

To a solution of 6-[(3-{3-[(tert-butyldimethylsilyl)oxy]prop-1-yn-1-yl}-2-methoxyphenyl)amino]-N-[(1R,2S)-2-fluorocyclopropyl]-8-{[(4-methoxyphenyl)methyl](methyl)amino}imidazo[1,2-b]pyridazine-3-carboxamide (1 g, 2 mmol) in DCM (8 mL) was added TFA (4 mL) dropwise at rt. The reaction mixture was stirred for 2 h at rt under nitrogen atmosphere. On completion, the mixture was concentrated under reduced pressure. The residue was triturated with Et₂O to afford the title compound (400 mg, 62.09%) as an off-white solid. LC/MS (ESI, m/z): [(M+H)]⁺=425.2.

### Step 4—3-{3-[(3-{[(1R,2S)-2-fluorocyclopropyl]carbamoyl}-8-(methylamino)imidazo[1,2-b]pyridazin-6-yl)amino]-2-methoxyphenyl}prop-2-yn-1-yl Methanesulfonate

To a stirred solution of N-[(1R,2S)-2-fluorocyclopropyl]-6-{[3-(3-hydroxyprop-1-yn-1-yl)-2-methoxyphenyl]amino}-8-(methylamino)imidazo[1,2-b]pyridazine-3-carboxamide (300 mg, 0.7 mmol) and methanesulfonyl methanesulfonate (184.69 mg, 1.060 mmol) in DCM (5 mL) were added TEA (0.29 mL, 2.12 mmol) and DMAP (8.64 mg, 0.071 mmol) at rt under nitrogen atmosphere. The resulting mixture was stirred for 2 h at rt under nitrogen atmosphere. On completion, the mixture was concentrated under reduced pressure to afford the title compound (350 mg, 99% yield) as a light yellow oil. LC/MS (ESI, m/z): [(M+H)]⁺=503.1.

### Example 1.278. Preparation of 6-{[6-fluoro-3-(5-formylpyridin-2-yl)-2-methoxyphenyl]amino}-N-[(1R,2S)-2-fluorocyclopropyl]-8-(methylamino)imidazo[1,2-b]pyridazine-3-carboxamide (Intermediate KG)

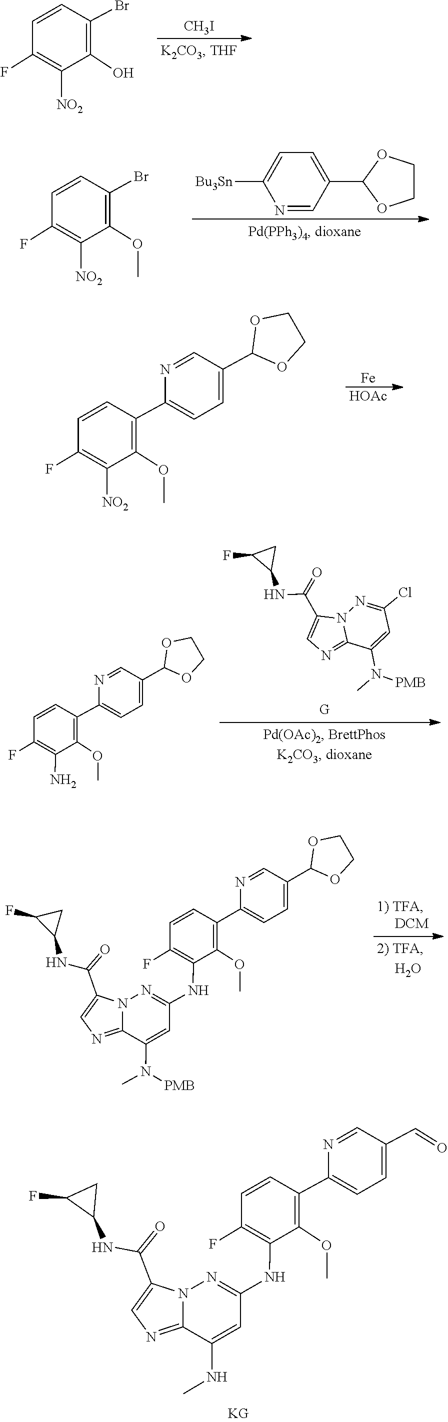

### Step 1—1-Bromo-4-fluoro-2-methoxy-3-nitrobenzene

To a stirred solution of 6-bromo-3-fluoro-2-nitrophenol (5.00 g, 21.2 mmol) and K₂CO₃ (8.78 g, 63.6 mmol) in THF (60 mL) was added CH₃I (6.01 g, 42.4 mmol) at 0° C. under nitrogen atmosphere. The resulting mixture was stirred for 16 h at rt under nitrogen atmosphere. On completion, the mixture was concentrated under reduced pressure. The residue was purified by silica gel column chromatography, eluted with PE/EA (5:1), to afford the title compound (3.02 g, 57% yield) as a yellow solid.

### Step 2—5-(1,3-Dioxolan-2-yl)-2-(4-fluoro-2-methoxy-3-nitrophenyl)pyridine

To a stirred solution of 1-bromo-4-fluoro-2-methoxy-3-nitrobenzene (2.00 g, 8.00 mmol) and 5-(1,3-dioxolan-2-yl)-2-(tributylstannyl)pyridine (3.52 g, 8.00 mmol, Intermediate JX) in toluene (20 mL) was added Pd(PPh₃)₄ (0.92 g, 0.80 mmol) at rt under nitrogen atmosphere. The resulting mixture was stirred for 2 h at 100° C. under nitrogen atmosphere. On completion, the mixture was cooled to rt and concentrated under reduced pressure. The residue was purified by silica gel column chromatography, eluted with PE/EA (3:1), to afford the title compound (650 mg, 25% yield) as a yellow solid. LC/MS (ESI, m/z): [(M+H)]⁺=321.0.

### Step 3—3-[5-(1,3-Dioxolan-2-yl)pyridin-2-yl]-6-fluoro-2-methoxyaniline

To a stirred solution of 5-(1,3-dioxolan-2-yl)-2-(4-fluoro-2-methoxy-3-nitrophenyl)pyridine (600 mg, 2 mmol) in HOAc (10 mL) was added Fe powder (735 mg, 13.2 mmol) at rt under nitrogen atmosphere. The resulting mixture was stirred for 2 h at rt under nitrogen atmosphere. On completion, the mixture was basified to pH 8 with saturated Na₂CO₃ (aq.). The resulting mixture was extracted with EtOAc (20 mL). The combined organic layers were washed with EtOAc (3×10 mL), and dried over anhydrous Na₂SO₄. After filtration, the filtrate was concentrated under reduced pressure. The resulting mixture was concentrated under reduced pressure. The residue was purified by silica gel column chromatography, eluted with PE/EA (1:1), to afford the title compound (506 mg, 93% yield) as a black oil. LC/MS (ESI, m/z): [(M+H)]⁺=291.1.

### Step 4—6-({3-[5-(1,3-Dioxolan-2-yl)pyridin-2-yl]-6-fluoro-2-methoxyphenyl}amino)-N-[(1R,2S)-2-fluorocyclopropyl]-8-{[(4-methoxyphenyl)methyl](methyl)amino}imidazo[1,2-b]pyridazine-3-carboxamide

To a stirred solution of 3-[5-(1,3-dioxolan-2-yl)pyridin-2-yl]-6-fluoro-2-methoxyaniline (260 mg, 0.896 mmol) and 6-chloro-N-[(1R,2S)-2-fluorocyclopropyl]-8-{[(4-methoxyphenyl)methyl](methyl)amino}imidazo[1,2-b]pyridazine-3-carboxamide (362 mg, 0.896 mmol, Intermediate G) in dioxane (8 mL) was added K₂CO₃ (371 mg, 2.68 mmol), BrettPhos (96 mg, 0.18 mmol) and Pd(OAc)₂ (20 mg, 0.09 mmol) at rt under nitrogen atmosphere. The resulting mixture was stirred for 2 h at 100° C. under nitrogen atmosphere. On completion, the mixture was cooled to rt and concentrated under reduced pressure. The residue was purified by reverse flash chromatography (column, C18 silica gel; mobile phase A: water (10 mmol/L NH₄HCO₃), mobile phase B: ACN, 50% to 70% gradient in 25 min; detector, UV 254 nm; the fractions were collected at 59%) to afford the title compound (234 mg, 40% yield) as a yellow solid. LC/MS (ESI, m/z): [(M+H)]⁺=658.5.

### Step 5—6-{[6-Fluoro-3-(5-formylpyridin-2-yl)-2-methoxyphenyl]amino}-N-[(1R,2S)-2-fluorocyclopropyl]-8-(methylamino)imidazo[1,2-b]pyridazine-3-carboxamide

To a solution of 6-({3-[5-(1,3-dioxolan-2-yl)pyridin-2-yl]-6-fluoro-2-methoxyphenyl}amino)-N-[(1R,2S)-2-fluorocyclopropyl]-8-{[(4-methoxyphenyl)methyl](methyl)amino}imidazo[1,2-b]pyridazine-3-carboxamide (300 mg, 0.5 mmol) in DCM (3 mL) was added TFA (1 mL) at rt. The resulting mixture was stirred for 1 h at rt under nitrogen atmosphere. Then the resulting mixture was concentrated under reduced pressure. Then to the above mixture were added TFA (3 mL) and H₂O (3 mL) at rt. The resulting mixture was stirred for additional 1 h at rt. On completion, the mixture was basified to pH 8 with saturated Na₂CO₃ (aq.). The resulting mixture was extracted with EtOAc (3×20 mL). The combined organic layers were washed with brine (20 mL), dried and over anhydrous Na₂SO₄. After filtration, the filtrate was concentrated under reduced pressure to afford the title compound (200 mg, 89% yield) as a yellow solid. LC/MS (ESI, m/z): [(M+H)]⁺=494.2.

### Example 1.279. Preparation of N-[(1R,2S)-2-fluorocyclopropyl]-8-(methylamino)-6-[4-(piperazin-1-yl)-2,3-dihydroindol-1-yl]imidazo[1,2-b]pyridazine-3-carboxamide (Intermediate KH)

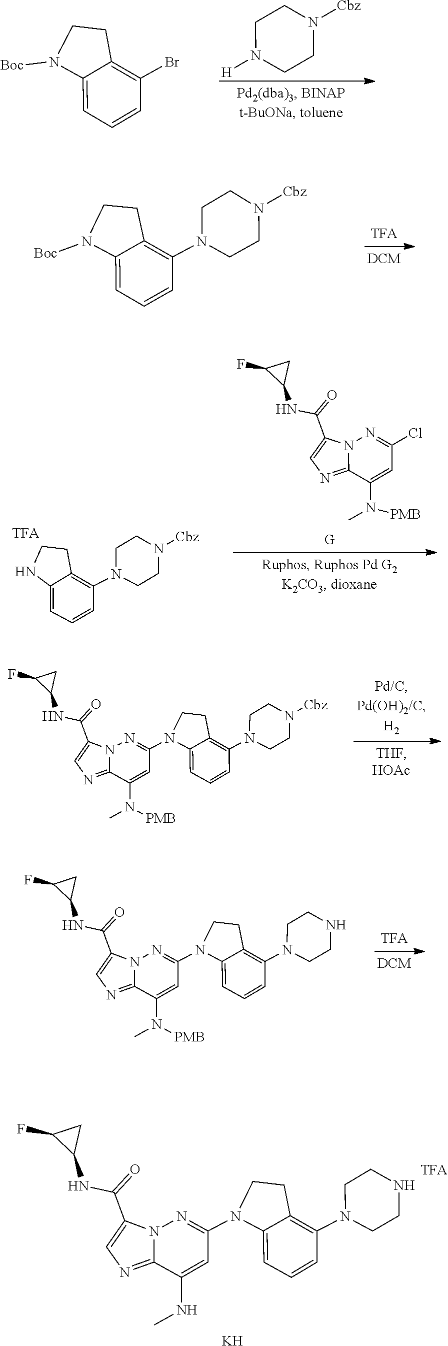

### Step 1—Tert-butyl 4-{4-[(benzyloxy)carbonyl]piperazin-1-yl}-2,3-dihydroindole-1-carboxylate

To a stirred solution of tert-butyl 4-bromo-2,3-dihydroindole-1-carboxylate (2.00 g, 6.71 mmol, CAS #885272-46-8) and benzyl piperazine-1-carboxylate (1.48 g, 6.71 mmol) in toluene (20 mL) was added BINAP (0.84 g, 1.3 mmol), t-BuONa (1.93 g, 20.1 mmol) and Pd₂(dba)₃ (0.61 g, 0.67 mmol) at rt under nitrogen atmosphere. The resulting mixture was stirred for 2 h at 110° C. under nitrogen atmosphere. On completion, the mixture was cooled to rt and concentrated under reduced pressure. The residue was purified by silica gel column chromatography, eluted with PE/EA (1:1), to afford the title compound (1.2 g, 41% yield) as a yellow oil. LC/MS (ESI, m/z): [(M+H)]⁺=438.2.

### Step 2—Benzyl 4-(2,3-dihydro-1H-indol-4-yl)piperazine-1-carboxylate Trifluoroacetate

A solution of tert-butyl 4-{4-[(benzyloxy)carbonyl]piperazin-1-yl}-2,3-dihydroindole-1-carboxylate (1.2 g, 2.7 mmol) in DCM (10 mL) was added TFA (5 mL) and stirred for 1 h at rt under nitrogen atmosphere. On completion, the mixture was concentrated under reduced pressure. The residue was purified by trituration with ethyl ether to afford the title compound (1.29 g) as a yellow oil. LC/MS (ESI, m/z): [(M+H)]⁺=338.0.

### Step 3—Benzyl 4-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]piperazine-1-carboxylate

To a stirred solution of benzyl 4-(2,3-dihydro-1H-indol-4-yl)piperazine-1-carboxylate trifluoroacetate (1.29 g, 2.96 mmol) and 6-chloro-N-[(1R,2S)-2-fluorocyclopropyl]-8-{[(4-methoxyphenyl)methyl](methyl)amino}imidazo[1,2-b]pyridazine-3-carboxamide (1.20 g, 2.96 mmol, Intermediate G) in dioxane (10 mL) was added K₂CO₃ (1.23 g, 8.89 mmol), RuPhos (0.28 g, 0.59 mmol) and 2nd Generation RuPhos precatalyst (0.23 g, 0.30 mmol) at rt under nitrogen atmosphere. The resulting mixture was stirred for 2 h at 100° C. under nitrogen atmosphere. On completion, the mixture was cooled to rt and was concentrated under reduced pressure. The residue was purified by silica gel column chromatography, eluted with DCM/EA (1:4), to afford the title compound (920 mg, 44% yield) as a yellow oil. LC/MS (ESI, m/z): [(M+H)]⁺=705.5.

### Step 4—N-[(1R,2S)-2-fluorocyclopropyl]-8-{[(4-methoxyphenyl)methyl](methyl)amino}-6-[4-(piperazin-1-yl)-2,3-dihydroindol-1-yl]imidazo[1,2-b]pyridazine-3-carboxamide

To a stirred solution of benzyl 4-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]piperazine-1-carboxylate (920 mg, 1.3 mmol) in THF (10 mL) and HOAc (0.1 mL) were added Pd/C (50 mg, 0.5 mmol) and Pd(OH)₂ (55 mg, 0.39 mmol) at rt under nitrogen atmosphere. The resulting mixture was purged with hydrogen three times and stirred for 2 h at rt under hydrogen atmosphere. On completion, the mixture was filtered, the filter cake was washed with THF (3×5 mL). The filtrate was concentrated under reduced pressure to afford the title compound (800 mg) as a white solid. LC/MS (ESI, m/z): [(M+H)]⁺=571.4.

### Step 5—N-[(1R,2S)-2-fluorocyclopropyl]-8-(methylamino)-6-[4-(piperazin-1-yl)-2,3-dihydroindol-1-yl]imidazo[1,2-b]pyridazine-3-carboxamide Trifluoroacetate

A solution of N-[(1R,2S)-2-fluorocyclopropyl]-8-{[(4-methoxyphenyl)methyl](methyl)amino}-6-[4-(piperazin-1-yl)-2,3-dihydroindol-1-yl]imidazo[1,2-b]pyridazine-3-carboxamide (800 mg, 1 mmol) in DCM (6 mL) was added TFA (3 mL) and stirred for 1 h at rt under nitrogen atmosphere. On completion, the resulting mixture was concentrated under reduced pressure. The residue was purified by trituration with Et₂O (10 mL) to afford the title compound (700 mg) as a white solid. LC/MS (ESI, m/z): [(M+H)]⁺=451.2.

### Example 1.280. Preparation of 6-(3-{[3-(1-isopropyl-1,2,3-triazol-4-yl)-8-(methylamino)imidazo[1,2-b]pyridazin-6-yl]amino}phenyl)pyridine-3-carbaldehyde (Intermediate KI)

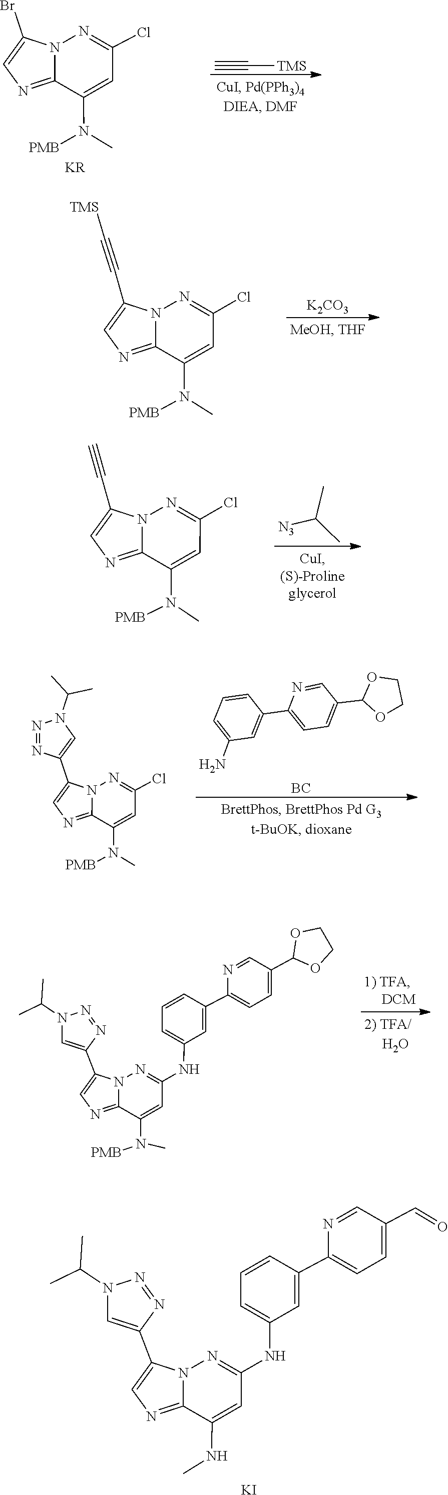

### Step 1—6-Chloro-N-[(4-methoxyphenyl)methyl]-N-methyl-3-[2-(trimethylsilyl)ethynyl]imidazo[1,2-b]pyridazin-8-amine

To a stirred mixture of 6-chloro-3-iodo-N-[(4-methoxyphenyl)methyl]-N-methylimidazo[1,2-b]pyridazin-8-amine (6.24 g, 14.6 mmol, Intermediate KR) and trimethylsilylacetylene (2.14 g, 21.8 mmol) in DMF (60 mL) were added Pd(PPh₃)₄ (0.84 g, 0.73 mmol) and CuI (0.14 g, 0.73 mmol) in portions at rt under nitrogen atmosphere. To the above mixture was added DIEA (4.72 g, 29.1 mmol) in portions at rt. The resulting mixture was stirred for additional 16 h at 90° C. On completion, the mixture was cooled to rt and the residue was purified by silica gel column chromatography, eluted with PE/EA (5:1) to afford the title compound (2.5 g, 43% yield) as a white solid. LC/MS (ESI, m/z): [(M+H)]⁺=399.1.

### Step 2—6-Chloro-3-ethynyl-N-[(4-methoxyphenyl)methyl]-N-methylimidazo[1,2-b]pyridazin-8-amine

To a stirred mixture of 6-chloro-N-[(4-methoxyphenyl)methyl]-N-methyl-3-[2-(trimethylsilyl)ethynyl]imidazo[1,2-b]pyridazin-8-amine (2.5 g, 6.3 mmol) in MeOH (25 mL) was added K₂CO₃ (1.297 g, 9.40 mmol) in portions at rt. The resulting mixture was stirred for 1 h at rt. On completion, the mixture was filtered, and the filter cake was washed with MeOH (1×20 mL). The filtrate was concentrated under reduced pressure. The residue was purified by silica gel column chromatography, eluted with PE/EA (30:1), to afford the title compound (650 mg, 32% yield) as a white solid. LC/MS (ESI, m/z): [(M+H)]⁺=327.2.

### Step 3—6-Chloro-3-(1-isopropyl-1,2,3-triazol-4-yl)-N-[(4-methoxyphenyl)methyl]-N-methylimidazo[1,2-b]pyridazin-8-amine

To a stirred solution of 6-chloro-3-ethynyl-N-[(4-methoxyphenyl)methyl]-N-methylimidazo[1,2-b]pyridazin-8-amine (600 mg, 2 mmol) and 2-azidopropane (187.52 mg, 2.203 mmol) in ACN (4 mL) and DMF (12 mL) were added CuI (34.97 mg, 0.184 mmol) and TEA (0.500 mL, 3.67 mmol) at rt under nitrogen atmosphere. The resulting mixture was stirred for 3 h at 80° C. under nitrogen then cooled and concentrated under reduced pressure. The residue was purified by reverse flash chromatography (column, silica gel; mobile phase, MeCN in water, 5% to 5% gradient in 6 min, 35% to 70% gradient in 30 min; detector, UV 254 nm) to afford the title compound (600 mg, 79% yield). LC/MS (ESI, m/z): [(M+H)]⁺=412.1.

### Step 4—N6-{3-[5-(1,3-dioxolan-2-yl)pyridin-2-yl]phenyl}-3-(1-isopropyl-1,2,3-triazol-4-yl)-N8-[(4-methoxyphenyl)methyl]-N8-methylimidazo[1,2-b]pyridazine-6,8-diamine

To a stirred solution of 6-chloro-3-(1-isopropyl-1,2,3-triazol-4-yl)-N-[(4-methoxyphenyl)methyl]-N-methylimidazo[1,2-b]pyridazin-8-amine (800 mg, 2 mmol) and 3-[5-(1,3-dioxolan-2-yl)pyridin-2-yl]aniline (470.57 mg, 1.942 mmol, Intermediate BC) in dioxane (30 mL) were added BrettPhos Pd G3 (176.07 mg, 0.194 mmol) and BrettPhos (104.26 mg, 0.194 mmol), sodium 2-methylpropan-2-olate (373.32 mg, 3.884 mmol) at rt under nitrogen atmosphere. The resulting mixture was stirred for additional 3 h at 110° C. On completion, the mixture was cooled to rt and concentrated under reduced pressure. The residue was purified by reverse phase flash chromatography (Column: WelFlash™ C18-I, 20-40 um, 330 g; Eluent A: Water (plus 10 mmol/L NH4HCO3); Eluent B: ACN; Gradient: 25%-55% B in 25 min; Flow rate: 80 mL/min; Detector: 220/254 nm; desired fractions were collected at 50% B) to afford the title compound (200 mg, 17% yield) as a yellow solid. LC/MS (ESI, m/z): [(M+H)]⁺=618.2.

### Step 5—6-(3-{[3-(1-Isopropyl-1,2,3-triazol-4-yl)-8-(methylamino)imidazo[1,2-b]pyridazin-6-yl]amino}phenyl)pyridine-3-carbaldehyde

To a stirred solution of N6-{3-[5-(1,3-dioxolan-2-yl)pyridin-2-yl]phenyl}-3-(1-isopropyl-1,2,3-triazol-4-yl)-N8-[(4-methoxyphenyl)methyl]-N8-methylimidazo[1,2-b]pyridazine-6,8-diamine (200 mg, 0.3 mmol) in DCM (3 mL) was added TFA (0.5 mL) at rt under nitrogen atmosphere. The resulting mixture was stirred for additional 2 h at rt. Then, the mixture was concentrated under reduced pressure. To the above mixture was added 2 M HCl (5 mL) at rt. The resulting mixture was stirred for additional 2 h at 60° C. On completion, the mixture was concentrated under reduced pressure. The residue was triturated with Et₂O to afford the title compound (50 mg, 34% yield) as a yellow solid. LC/MS (ESI, m/z): [(M+H)]⁺=454.2.

### Example 1.281. Preparation of N-[(1R,2S)-2-fluorocyclopropyl]-6-[1-methyl-7-(piperidin-4-yl)indol-3-yl]-8-(methylamino)imidazo[1,2-b]pyridazine-3-carboxamide (Intermediate KJ)

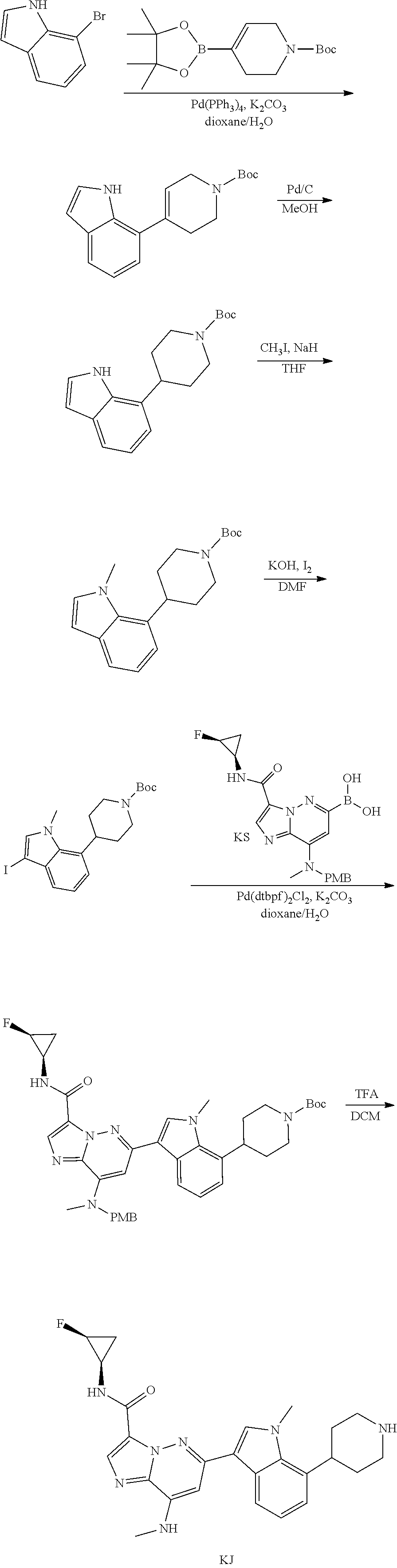

### Step 1—Tert-butyl 4-(1H-indol-7-yl)-3,6-dihydro-2H-pyridine-1-carboxylate

To a stirred solution of 7-bromo-1H-indole (5.00 g, 25.504 mmol) and tert-butyl 4-(4,4,5,5-tetramethyl-1,3,2-dioxaborolan-2-yl)-3,6-dihydro-2H-pyridine-1-carboxylate (9.46 g, 30.6 mmol, CAS #286961-14-6) in dioxane (60 mL) and H₂O (10 mL) were added K₂CO₃ (10.57 g, 76.51 mmol) and Pd(PPh₃)₄ (2.95 g, 2.55 mmol) at rt under nitrogen atmosphere. The resulting mixture was purged with nitrogen three times and stirred for 3 h at 80° C. under nitrogen atmosphere. On completion, the mixture was cooled to rt and concentrated under vacuum. The residue was purified by silica gel column chromatography, eluted with PE/EA (3:1), to afford the title compound (4 g, 53% yield) as a brown solid. LC/MS (ESI, m/z): [(M+1)]⁺=299.2.

### Step 2—Tert-butyl 4-(1H-indol-7-yl)piperidine-1-carboxylate

To a stirred solution of tert-butyl 4-(1H-indol-7-yl)-3,6-dihydro-2H-pyridine-1-carboxylate (4.00 g, 13.4 mmol) in MeOH (50 mL) was added Pd/C (0.29 g, 2.7 mmol, 10 wt %) at rt under nitrogen atmosphere. The resulting mixture was purged with hydrogen three times and stirred for 1 h at rt under hydrogen atmosphere. The resulting mixture was filtered, the filter cake was washed with MeOH (3×5 mL). The filtrate was concentrated under reduced pressure to afford the title compound (3.00 g, 75% yield) as a light brown oil. LC/MS (ESI, m/z): [(M+1)]⁺=301.1.

### Step 3—Tert-butyl 4-(1-methylindol-7-yl)piperidine-1-carboxylate

To a stirred solution of tert-butyl 4-(1H-indol-7-yl)piperidine-1-carboxylate (3.00 g, 10.0 mmol) in THF (40 mL) was added NaH (0.8 g, 20 mmol, 60% dispersion in mineral oil) at 0° C. under nitrogen atmosphere. The resulting mixture was stirred for 30 min at 0° C. under nitrogen atmosphere. To the above mixture was added CH₃I (1.70 g, 12.0 mmol) dropwise over 5 min at 0° C. The resulting mixture was stirred for additional 16 h at rt. On completion, the reaction was quenched with water at 0° C. The resulting mixture was extracted with EtOAc (3×50 mL). The combined organic layers were washed with brine (1×50 mL), and dried over anhydrous Na₂SO₄. After filtration, the filtrate was concentrated under reduced pressure. The residue was purified by silica gel column chromatography, eluted with PE/EA (2:1), to afford the title compound (1.5 g, 48% yield) as a colorless solid. LC/MS (ESI, m/z): [(M+1)]⁺=315.2.

### Step 4—Tert-butyl 4-(3-iodo-1-methylindol-7-yl)piperidine-1-carboxylate

To a stirred solution of tert-butyl 4-(1-methylindol-7-yl)piperidine-1-carboxylate (1.50 g, 4.77 mmol) and KOH (0.80 g, 14 mmol) in DMF (20 mL) at rt under nitrogen atmosphere. The resulting mixture was stirred for 30 min at rt under nitrogen atmosphere. To the above mixture was added I₂ (2.42 g, 9.54 mmol) in portions over 5 min at rt. The resulting mixture was stirred for additional 4 h at rt. On completion, the reaction was quenched with water at rt. The resulting mixture was extracted with EtOAc (3×50 mL). The combined organic layers were washed with brine (1×100 mL), and dried over anhydrous Na₂SO₄. After filtration, the filtrate was concentrated under reduced pressure. The residue was purified by silica gel column chromatography, eluted with PE/EA (1:1), to afford the title compound (1.7 g, 81% yield) as a brown solid. LC/MS (ESI, m/z): [(M+1)]⁺=441.1.

### Step 5—Tert-butyl 4-[3-(3-{[(1R,2S)-2-fluorocyclopropyl]carbamoyl}-8-{[(4-methoxyphenyl)methyl](methyl)amino}imidazo[1,2-b]pyridazin-6-yl)-1-methylindol-7-yl]piperidine-1-carboxylate

To a stirred solution of tert-butyl 4-(3-iodo-1-methylindol-7-yl)piperidine-1-carboxylate (600 mg, 1 mmol) and 3-{[(1R,2S)-2-fluorocyclopropyl]carbamoyl}-8-{[(4-methoxyphenyl)methyl](methyl)amino}imidazo[1,2-b]pyridazin-6-ylboronic acid (563.07 mg, 1.363 mmol, Intermediate KS) in dioxane (10 mL) and H₂O (1 mL) were added K₂CO₃ (564.97 mg, 4.089 mmol) and Pd(DtBPF)Cl₂ (88.81 mg, 0.136 mmol) at rt under nitrogen atmosphere. The resulting mixture was purged with nitrogen three times and stirred for 3 h at 80° C. under nitrogen atmosphere. On completion, the mixture was cooled to rt and concentrated under vacuum. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 30%-55% B in 25 min; Flow rate: 80 mL/min; Detector: 220/254 nm; desired fractions were collected at 50% B) and concentrated under reduced pressure to afford the title compound (700 mg, 75% yield) as a brown solid. LC/MS (ESI, m/z): [(M+1)]⁺=682.4.

### Step 6—N-[(1R,2S)-2-fluorocyclopropyl]-6-[1-methyl-7-(piperidin-4-yl)indol-3-yl]-8-(methylamino)imidazo[1,2-b]pyridazine-3-carboxamide

To a stirred solution of tert-butyl 4-[3-(3-{[(1R,2S)-2-fluorocyclopropyl]carbamoyl}-8-{[(4-methoxyphenyl)methyl](methyl)amino}imidazo[1,2-b]pyridazin-6-yl)-1-methylindol-7-yl]piperidine-1-carboxylate (700 mg, 1 mmol) in DCM (6 mL) was added TFA (3 mL) at rt under nitrogen atmosphere. The resulting mixture was stirred for 1 h at rt under nitrogen atmosphere. On completion, the mixture was concentrated under vacuum. The residue was purified by reverse phase flash chromatography (Column: WelFlash™ C18-I, 20-40 μm, 330 g; Eluent A: Water (plus 10 mmol/L NH₄HCO₃); Eluent B: ACN; Gradient: 30%-55% B in 25 min; Flow rate: 80 mL/min; Detector: 220/254 nm; desired fractions were collected at 45% B) to afford the title compound (360 mg, 76% yield) as a white solid. LC/MS (ESI, m/z): [(M+1)]⁺=462.3; ¹H NMR (400 MHz, DMSO-d₆) δ 9.12 (d, J=4.6 Hz, 1H), 8.17 (s, 1H), 8.08-8.02 (m, 2H), 7.72 (q, J=4.9 Hz, 1H), 7.19 (t, J=7.6 Hz, 1H), 7.13 (d, J=7.3 Hz, 1H), 6.56 (s, 1H), 5.01-4.78 (m, 1H), 4.17 (s, 3H), 3.90-3.75 (m, 1H), 3.48-3.40 (m, 2H), 3.24-3.13 (m, 2H), 3.12-3.05 (m, 1H), 3.01 (d, J=4.9 Hz, 3H), 2.12-2.04 (m, 2H), 2.04-1.89 (m, 2H), 1.33-1.20 (m, 1H), 1.11-0.97 (m, 1H).

### Example 1.282. 1-[2-chloro-5-(piperazine-1-carbonyl)phenyl]-1,3-diazinane-2,4-dione (Intermediate KK)

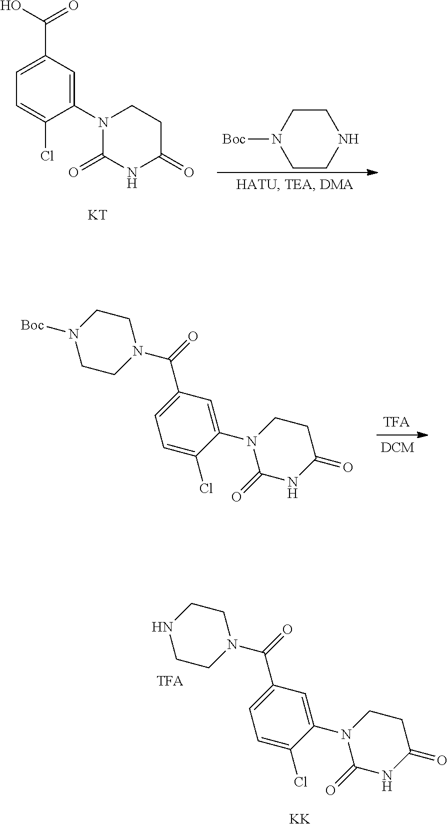

### Step 1—Tert-butyl 4-[4-chloro-3-(2,4-dioxo-1,3-diazinan-1-yl)benzoyl]piperazine-1-carboxylate

To a stirred solution of 4-chloro-3-(2,4-dioxo-1,3-diazinan-1-yl) benzoic acid (400 mg, 2 mmol, Intermediate KT) and tert-butyl piperazine-1-carboxylate (415.98 mg, 2.234 mmol) in DMA (10 mL) was added TEA (0.814 mL, 5.96 mmol) at rt under nitrogen atmosphere. To the above mixture was added HATU (679.37 mg, 1.787 mmol) at 0° C. The resulting mixture was stirred for additional 1 h at rt. On completion, the reaction solution was concentrated under reduced pressure. The residue was purified by reverse flash chromatography (column, C₁₈ silica gel; mobile phase, MeCN in Water (10 mmol/L NH₄HCO₃), 15% to 45% gradient in 30 min; detector, UV 254 nm, desired fractions were collected at 29% B) to afford the title compound (350 mg, 54% yield) as a white solid. LC/MS (ESI, m/z): [(M+H)]⁺=437.1.

### Step 2—1-[2-Chloro-5-(piperazine-1-carbonyl)phenyl]-1,3-diazinane-2,4-dione Trifluoroacetate

To a stirred solution of tert-butyl 4-[4-chloro-3-(2,4-dioxo-1,3-diazinan-1-yl)benzoyl]piperazine-1-carboxylate (300 mg, 0.7 mmol) in DCM (5 mL) was added TFA (1 mL) dropwise at rt under nitrogen atmosphere. The resulting mixture was stirred for 1 h at rt under nitrogen atmosphere. On completion, the mixture was concentrated under reduced pressure. The residue was purified by trituration with diethyl ether (20 mL) to afford the title compound (230 mg) as a white solid. LC/MS (ESI, m/z): [(M+H)]⁺=337.0.

### Example 1.282. 3-(3-methyl-2-oxo-5-(4-(piperidin-4-ylmethyl)piperazin-1-yl)-2,3-dihydro-1H-benzo[d]imidazol-1-yl)piperidine-2,6-dione (Intermediate KL)

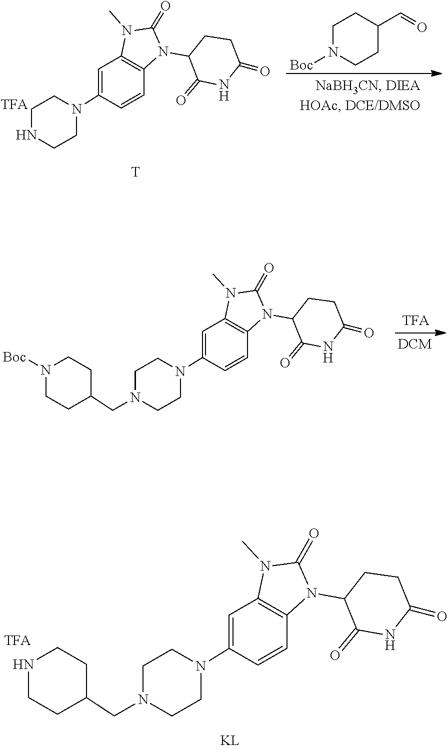

### Step 1—Tert-butyl 4-({4-[1-(2,6-dioxopiperidin-3-yl)-3-methyl-2-oxo-1,3-benzodiazol-5-yl]piperazin-1-yl}methyl)piperidine-1-carboxylate

To a stirred solution of 3-[3-methyl-2-oxo-5-(piperazin-1-yl)-1,3-benzodiazol-1-yl]piperidine-2,6-dione trifluoroacetate (1.4 g, 3.2 mmol, Intermediate T) and AcOK (0.93 g, 9.516 mmol) in DMSO (15 mL) and DCE (15 mL) were added tert-butyl 4-formylpiperidine-1-carboxylate (0.68 g, 3.2 mmol) and AcOH (0.10 g, 1.6 mmol). The resulting mixture was stirred for 1 h at rt under nitrogen atmosphere. To the above mixture was added NaBH₃CN (0.40 g, 6.344 mmol) at 0° C. The resulting mixture was then stirred for additional 16 h at rt. On completion, the mixture was concentrated under reduced pressure. The residue was purified by reverse flash chromatography (column, C18 silica gel; mobile phase, ACN in Water (0.1% FA), 15% to 40% gradient in 25 min; detector, UV 254 nm; desired fraction were collected at 23% B) to afford the title compound (700 mg, 41% yield) as a brown solid. LC/MS (ESI, m/z): [(M+H)]⁺=541.3.

### Step 2—3-{3-Methyl-2-oxo-5-[4-(piperidin-4-ylmethyl)piperazin-1-yl]-1,3-benzodiazol-1-yl}piperidine-2,6-dione Trifluoroacetate

To a stirred solution of tert-butyl 4-({4-[1-(2,6-dioxopiperidin-3-yl)-3-methyl-2-oxo-1,3-benzodiazol-5-yl]piperazin-1-yl}methyl)piperidine-1-carboxylate (1 g, 2 mmol) in DCM (20 mL) was added TFA (4 mL) at rt under nitrogen atmosphere. The resulting mixture was stirred for 4 h at rt under nitrogen atmosphere. On completion, the mixture was concentrated under reduced pressure. The residue was purified by trituration with diethyl ether (50 mL) to give the title compound (1 g) as a brown solid. LC/MS (ESI, m/z): [(M+H)]⁺=441.3.

### Example 1.283. Preparation of 3-(5-{3,9-diazaspiro[5.5]undecan-3-yl}-3-methyl-2-oxo-1,3-benzodiazol-1-yl)piperidine-2,6-dione (Intermediate KM)

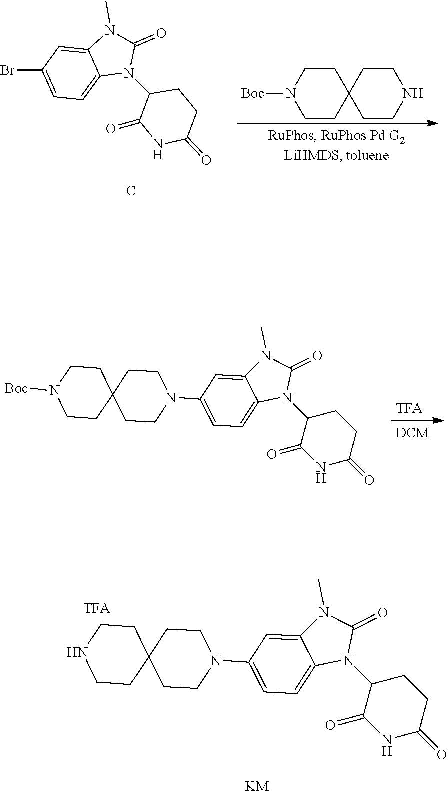

### Step 1—Tert-butyl 9-[1-(2,6-dioxopiperidin-3-yl)-3-methyl-2-oxo-1,3-benzodiazol-5-yl]-3,9-diazaspiro[5.5]undecane-3-carboxylate

To a stirred solution of 3-(5-bromo-3-methyl-2-oxo-1,3-benzodiazol-1-yl)piperidine-2,6-dione (2 g, 6 mmol, Intermediate C) and tert-butyl 3,9-diazaspiro[5.5]undecane-3-carboxylate (2.26 g, 8.87 mmol, CAS #173405-78-2) in toluene (20 mL) were added RuPhos (0.55 g, 1.2 mmol) and RuPhos-PdCl-2nd G (0.46 g, 0.59 mmol) at rt under nitrogen atmosphere. To the above mixture was added LiHMDS (35.49 mL, 35.48 mmol) dropwise at 0° C. The resulting mixture was stirred for additional 1 h at 100° C. On completion, the mixture was cooled to rt and the mixture was acidified to pH 5 with formic acid. The resulting mixture was concentrated under reduced pressure. The residue was purified by reverse flash chromatography (column, C18 silica gel; mobile phase, ACN in Water (0.1% FA), 20% to 40% gradient in 20 min; detector, UV 254 nm; desired fraction were collected at 34% B) to afford the title compound (1.7 g) as a black solid. LC/MS (ESI, m/z): [(M+1)]⁺=512.3.

### Step 2—3-(5-{3,9-Diazaspiro[5.5]undecan-3-yl}-3-methyl-2-oxo-1,3-benzodiazol-1-yl)piperidine-2,6-dione trifluoroacetate

To a stirred solution of tert-butyl 9-[1-(2,6-dioxopiperidin-3-yl)-3-methyl-2-oxo-1,3-benzodiazol-5-yl]-3,9-diazaspiro[5.5]undecane-3-carboxylate (700 mg, 1 mmol) in DCM (5 mL) was added TFA (5 mL) at rt under nitrogen atmosphere. The resulting mixture was stirred for 1 h at rt under nitrogen atmosphere. On completion, the mixture was concentrated under vacuum. The residue was purified by trituration with Et₂O (3 mL). The solids were collected by filtration and washed with Et₂O (2×2 mL) to afford the title compound (500 mg, 72% yield) as a brown solid. LC/MS (ESI, m/z): [(M+1)]⁺=412.2.

### Example 1.284. Preparation of 2,3-Dihydro-1H-indole-4-carbaldehyde (Intermediate KN)

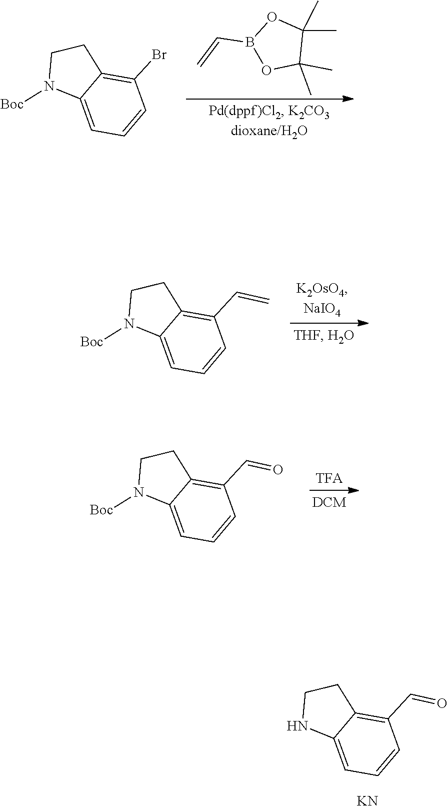

### Step 1—Tert-butyl 4-ethenyl-2,3-dihydroindole-1-carboxylate

To a stirred mixture of tert-butyl 4-bromo-2,3-dihydroindole-1-carboxylate (4.5 g, 15 mmol, CAS #885272-46-8) and 2-ethenyl-4,4,5,5-tetramethyl-1,3,2-dioxaborolane (4.65 g, 30.2 mmol) in H₂O (8 mL) and dioxane (40 mL) were added K₂CO₃ (6.26 g, 45.3 mmol) and Pd(dppf)Cl₂·CH₂Cl₂ (1.23 g, 1.51 mmol) at rt under nitrogen atmosphere. The resulting mixture was stirred for 2 h at 90° C. On completion, the mixture was cooled to rt and concentrated under reduced pressure. The residue was purified by silica gel column chromatography, eluted with PE/EA (5:1), to afford the title compound (2 g, 54% yield) as a light yellow solid. LC/MS (ESI, m/z): [(M−56+H)]⁺=190.1.

### Step 2—Tert-butyl 4-formyl-2,3-dihydroindole-1-carboxylate

To a stirred solution of tert-butyl 4-ethenyl-2,3-dihydroindole-1-carboxylate (2.3 g, 9.4 mmol) and NaIO₄ (10 g, 50 mmol) in THF (30 mL) and H₂O (15 mL) was added K₂OsO₄·2H₂O (173 mg, 0.470 mmol) at rt. The resulting mixture was stirred for 2 h at rt under nitrogen atmosphere. On completion, the mixture was extracted with EtOAc (3×30 mL). The combined organic layers were washed with brine (1×50 mL), and dried over anhydrous Na₂SO₄. After filtration, the filtrate was concentrated under reduced pressure to give the title compound (925 mg, 40% yield) as an off-white solid. LC/MS (ESI, m/z): [(M+H)]⁺=248.4.

### Step 3—2,3-Dihydro-1H-indole-4-carbaldehyde

To a solution of tert-butyl 4-formyl-2,3-dihydroindole-1-carboxylate (925 mg, 3.74 mmol) in DCM (14 mL) was added TFA (7 mL) dropwise at rt. The reaction mixture was stirred for 1 h at rt under nitrogen atmosphere. On completion, the mixture was basified to pH 8 with saturated Na₂CO₃ (aq.). The resulting mixture was extracted with CH₂Cl₂ (3×50 mL). The combined organic layers were washed with brine (1×50 mL), and dried over anhydrous Na₂SO₄. After filtration, the filtrate was concentrated under reduced pressure to afford the title compound (480 mg, 87% yield) as a brown yellow solid. LC/MS (ESI, m/z): [(M+H)]⁺=148.2.

### Example 1.285. Preparation of N-[(1R,2S)-2-fluorocyclopropyl]-6-(4-formyl-2,3-dihydroindol-1-yl)-8-(methylamino)imidazo[1,2-b]pyridazine-3-carboxamide (Intermediate KO)

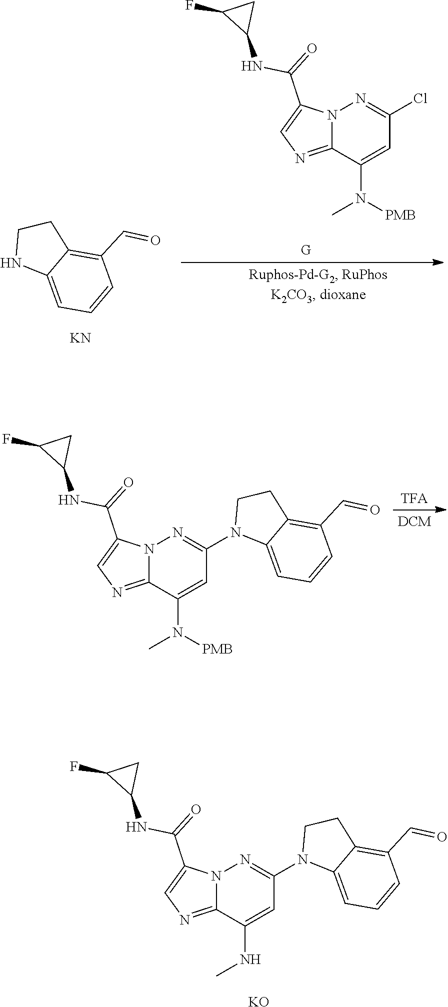

### Step 1—N-[(1R,2S)-2-fluorocyclopropyl]-6-(4-formyl-2,3-dihydroindol-1-yl)-8-{[(4-methoxyphenyl)methyl](methyl)amino}imidazo[1,2-b]pyridazine-3-carboxamide

To a stirred solution of 6-chloro-N-[(1R,2S)-2-fluorocyclopropyl]-8-{[(4-methoxyphenyl)methyl](methyl)amino}imidazo[1,2-b]pyridazine-3-carboxamide (1.37 g, 3.39 mmol, Intermediate G) and 2,3-dihydro-1H-indole-4-carbaldehyde (1.00 g, 6.78 mmol, Intermediate KN) in dioxane (7 mL) were added K₂CO₃ (1.41 g, 10.2 mmol), RuPhos (320 mg, 0.69 mmol) and RuPhos-PdCl-2nd G (260 mg, 0.33 mmol) at rt under nitrogen atmosphere. The resulting mixture was stirred for 4 h at 100° C. under nitrogen atmosphere. On completion, the mixture was cooled to rt and concentrated under reduced pressure. The residue was purified by reversed-phase flash chromatography (column, C18 silica gel; mobile phase, MeCN in Water (plus 10 mmol/L FA), 40% to 70% gradient in 30 min; detector, UV 254 nm) to afford the title compound (630 mg, 36% yield) as a brown solid. LC/MS (ESI, m/z): [(M+H)]⁺=515.3.

### Step 2—N-[(1R,2S)-2-fluorocyclopropyl]-6-(4-formyl-2,3-dihydroindol-1-yl)-8-(methylamino)imidazo[1,2-b]pyridazine-3-carboxamide

To a solution of N-[(1R,2S)-2-fluorocyclopropyl]-6-(4-formyl-2,3-dihydroindol-1-yl)-8-{[(4-methoxyphenyl)methyl](methyl)amino}imidazo[1,2-b]pyridazine-3-carboxamide (100 mg, 0.2 mmol) in DCM (2 mL) was added TFA (1 mL) dropwise at rt. The reaction mixture was stirred for 1 h at rt under nitrogen atmosphere. On completion, the mixture was concentrated under reduced pressure. The residue was triturated with Et₂O to afford the title compound (30.5 mg, 40% yield) as an off-white solid. LC/MS (ESI, m/z): [(M+H)]⁺=395.2.

### Example 1.286. Preparation of 1-(1-(2,6-dioxopiperidin-3-yl)-3-methyl-2-oxo-2,3-dihydro-1H-benzo[d]imidazol-4-yl)piperidine-4-carbaldehyde (Intermediate KP)

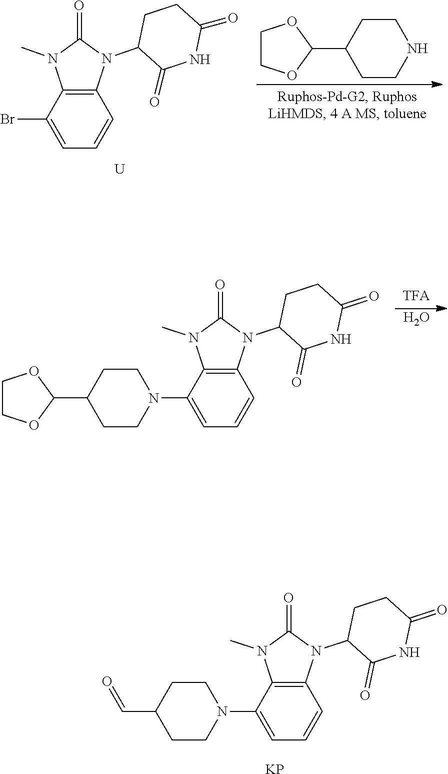

### Step 1—3-(4-(4-(1,3-Dioxolan-2-yl)piperidin-1-yl)-3-methyl-2-oxo-2,3-dihydro-1H-benzo[d]imidazol-1-yl)piperidine-2,6-dione

To a stirred solution of 3-(4-bromo-3-methyl-2-oxo-2,3-dihydro-1H-benzo[d]imidazol-1-yl)piperidine-2,6-dione (5 g, 15 mmol, Intermediate U) and 4-(1,3-dioxolan-2-yl)piperidine (2.32 g, 14.8 mmol) in toluene (200 mL) was added LiHMDS (88.72 mL, 88.71 mmol) dropwise over 10 min at 0° C. under argon atmosphere. The resulting mixture was stirred for 30 min at 0° C. under argon atmosphere. To the above mixture was added RuPhos (689.98 mg, 1.479 mmol) and RuPhos-PdCl-2nd G (1.15 g, 1.48 mmol) in portions at 0° C. The resulting mixture was stirred for additional 1 h at 80° C. The reaction was monitored by LCMS. On completion, the mixture was cooled to rt and acidified to pH 5˜6 with HCOOH. The resulting mixture was concentrated under reduced pressure. The residue was purified by silica gel column chromatography, eluted with CH2Cl2/MeOH (10:1), to afford the title compound (4.5 g, 73% yield) as a grey solid. LC/MS (ESI, m/z): [(M+H)]⁺=415.3.

### Step 2—1-[1-(2,6-Dioxopiperidin-3-yl)-3-methyl-2-oxo-1,3-benzodiazol-4-yl]piperidine-4-carbaldehyde

To a stirred mixture of 3-{4-[4-(1,3-dioxolan-2-yl)piperidin-1-yl]-3-methyl-2-oxo-1,3-benzodiazol-1-yl}piperidine-2,6-dione (1 g, 2 mmol) in H₂O (10 mL) was added TFA (2 mL) dropwise at rt under nitrogen atmosphere. The resulting mixture was stirred for 6 h at 50° C. under nitrogen atmosphere. On completion, the mixture was cooled to rt and basified to pH 9 with DIEA. The residue was purified by reverse flash chromatography (column, C18 silica gel; mobile phase, MeCN in Water (10 mmol/L NH₄HCO₃), 15% to 55% gradient in 10 min; detector, UV 254 nm) to afford (400 mg, 45% yield) as a brown solid. LC/MS (ESI, m/z): [(M+H)]⁺=371.2.

### Example 1.287. Preparation of N-[(1R,2S)-2-fluorocyclopropyl]-6-[4-(4-fluoropyridin-2-yl)-2,3-dihydroindol-1-yl]-8-{[(4-methoxyphenyl)methyl](methyl)amino}imidazo[1,2-b]pyridazine-3-carboxamide (Intermediate KO)

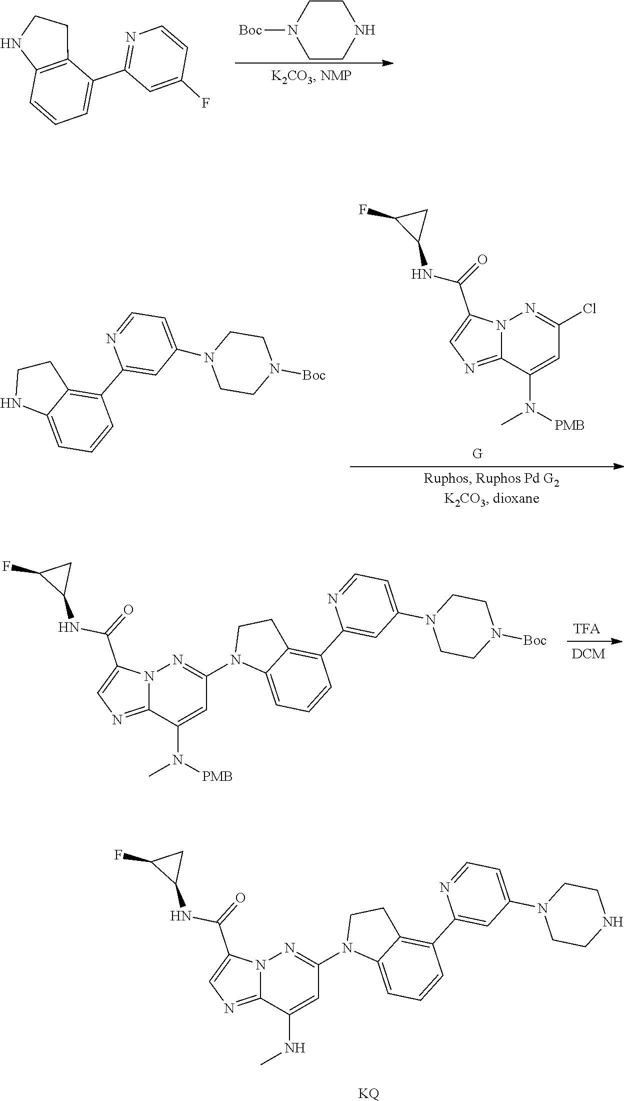

### Step 1—Tert-butyl 4-[2-(2,3-dihydro-1H-indol-4-yl)pyridin-4-yl]piperazine-1-carboxylate

To a stirred solution of 4-(4-fluoropyridin-2-yl)-2,3-dihydro-1H-indole (0.9 g, 4 mmol, synthesized via Steps 1-2 of Intermediate JZ) and tert-butyl piperazine-1-carboxylate (0.86 g, 4.6 mmol) in NMP (15 mL) was added K₂CO₃ (1.74 g, 12.6 mmol) at rt. The resulting mixture was stirred for 16 h at 100° C. On completion, the mixture was cooled to rt. The residue was purified by reversed-phase flash chromatography (column, C18 silica gel; mobile phase, MeCN in water (10 mmol/L NH₄HCO₃), 5% to 70% gradient in 30 min; detector, UV 254 nm) to afford the title compound (0.7 g, 44% yield) as a light yellow solid. LC/MS (ESI, m/z): [(M+H)]⁺=381.2.

### Step 2—N-[(1R,2S)-2-fluorocyclopropyl]-6-[4-(4-fluoropyridin-2-yl)-2,3-dihydroindol-1-yl]-8-{[(4-methoxyphenyl)methyl](methyl)amino}imidazo[1,2-b]pyridazine-3-carboxamide

To a stirred mixture of 4-(4-fluoropyridin-2-yl)-2,3-dihydro-1H-indole (500 mg, 2 mmol), 6-chloro-N-[(1R,2S)-2-fluorocyclopropyl]-8-{[(4-methoxyphenyl)methyl](methyl)amino}imidazo[1,2-b]pyridazine-3-carboxamide (1.04 g, 2.57 mmol, Intermediate G) and K₂CO₃ (967.63 mg, 7.002 mmol) in dioxane (20 mL) were added RuPhos (108.91 mg, 0.233 mmol) and RuPhos-PdCl-2nd G (181.51 mg, 0.233 mmol) at rt under nitrogen atmosphere. The resulting mixture was then stirred for 16 h at 100° C. under nitrogen atmosphere. On completion, the mixture was cooled to rt and concentrated under reduced pressure. The residue was purified by silica gel column chromatography, eluted with PE/EA (100%), to afford the title compound (730 mg, 54% yield) as a light yellow solid. LC/MS (ESI, m/z): [(M+H)]⁺=748.4.

### Step 3—N-[(1R,2S)-2-fluorocyclopropyl]-6-[4-(4-fluoropyridin-2-yl)-2,3-dihydroindol-1-yl]-8-{[(4-methoxyphenyl)methyl](methyl)amino}imidazo[1,2-b]pyridazine-3-carboxamide

To a stirred solution of tert-butyl 4-{2-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]pyridin-4-yl}piperazine-1-carboxylate (800 mg, 1 mmol) in DCM (6 mL) was added TFA (2 mL) dropwise at rt. The resulting mixture was stirred for 1 h at rt. On completion, the mixture was concentrated under reduced pressure. The residue was triturated by Et₂O to afford the title compound (0.55 g, 97% yield) as a brown semi-solid. LC/MS (ESI, m/z): [(M+H)]⁺=582.3.

### Example 1.288. Preparation of 3-bromo-6-chloro-N-[(4-methoxyphenyl)methyl]-N-methylimidazo[1,2-b]pyridazin-8-amine (Intermediate KR)

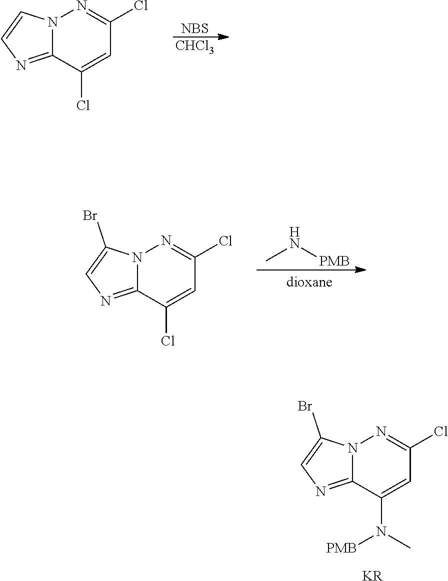

### Step 1—3-Bromo-6,8-dichloroimidazo[1,2-b]pyridazine

To a stirred solution of 6,8-dichloroimidazo[1,2-b]pyridazine (38 g, 200 mmol, CAS #1161847-29-5) in CHCl₃ (500 mL) was added NBS (53.96 g, 303.2 mmol) at rt under nitrogen atmosphere. The resulting mixture was stirred for 16 h at rt under nitrogen atmosphere. On completion, the mixture was filtered, and the filter cake was washed with CHCl₃ (3×20 mL). The filtrate was concentrated under reduced pressure to afford the title compound (49 g) as a black solid. LC/MS (ESI, m/z): [(M+H)]⁺=267.9.

### Step 2—3-Bromo-6-chloro-N-[(4-methoxyphenyl)methyl]-N-methylimidazo[1,2-b]pyridazin-8-amine

To a stirred solution of 3-bromo-6,8-dichloroimidazo[1,2-b]pyridazine (49 g, 183.582 mmol) and [(4-methoxyphenyl)methyl](methyl)amine (41.64 g, 275.4 mmol) in dioxane (500 mL) was added K₂CO₃ (50.74 g, 367.2 mmol) at rt under nitrogen atmosphere. The resulting mixture was stirred for 16 h at rt under nitrogen atmosphere. On completion, the mixture was filtered, and the filter cake was washed with dioxane (3×10 mL). The filtrate was concentrated under reduced pressure. The residue was purified by trituration with diethyl ether (500 mL). The resulting mixture was filtered, and the filter cake was washed with diethyl ether (3×10 mL). The collected solid was dried under reduced pressure to afford the title compound (60 g, 86% yield) as a brown solid. LC/MS (ESI, m/z): [(M+H)]⁺=381.1.

### Example 1.289. Preparation of 3-{[(1R,2S)-2-fluorocyclopropyl]carbamoyl}-8-{[(4-methoxyphenyl)methyl](methyl)amino}imidazo[1,2-b]pyridazin-6-ylboronic acid (Intermediate KS)

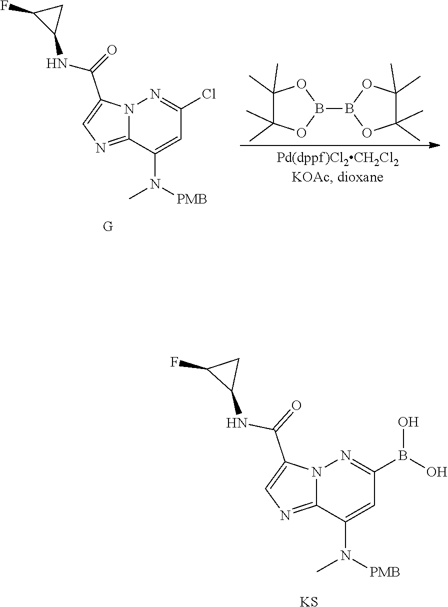

To a stirred solution of 6-chloro-N-[(1R,2S)-2-fluorocyclopropyl]-8-{[(4-methoxyphenyl)methyl](methyl)amino}imidazo[1,2-b]pyridazine-3-carboxamide (5 g, 10 mmol, Intermediate G) and 4,4,5,5-tetramethyl-2-(tetramethyl-1,3,2-dioxaborolan-2-yl)-1,3,2-dioxaborolane (4.72 g, 18.6 mmol) in dioxane (50 mL) were added KOAc (3.65 g, 37.1 mmol) and Pd(dppf)Cl₂CH₂Cl₂ (1.01 g, 1.24 mmol) at rt under nitrogen atmosphere. The resulting mixture was purged with nitrogen three times and stirred for 16 h at 90° C. under nitrogen atmosphere. On completion, the mixture was cooled to rt and concentrated under reduced pressure. The residue was purified by reverse phase flash chromatography (Column: WelFlash™ C18-I, 20-40 μm, 330 g; Eluent A: Water (plus 10 mmol/L FA); Eluent B: ACN; Gradient: 20%-50% B in 30 min; Flow rate: 80 mL/min; Detector: 220/254 nm; desired fractions were collected at 40% B) to afford the title compound (3.8 g, 74% yield) as a white solid. LC/MS (ESI, m/z): [(M+H)]⁺=414.2.

### Example 1.290. Preparation of 4-Chloro-3-(2,4-dioxo-1,3-diazinan-1-yl)benzoic Acid (Intermediate KT)

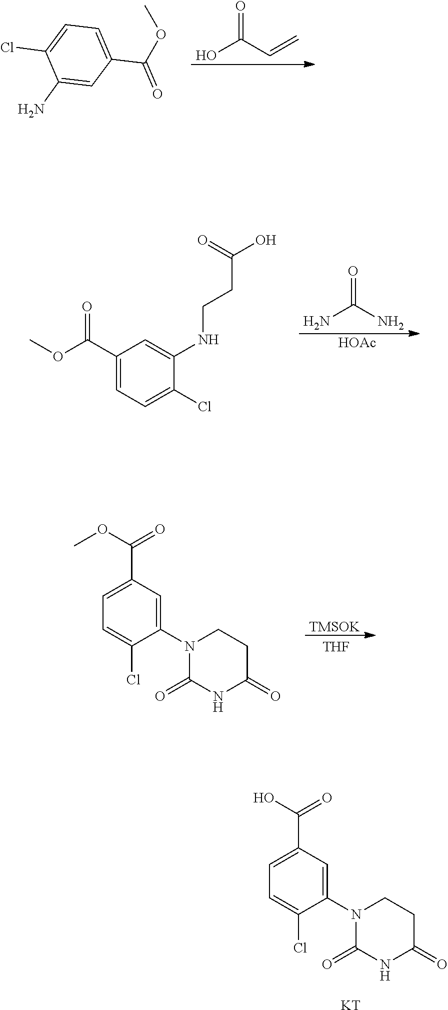

### Step 1—3-{[2-Chloro-5-(methoxycarbonyl)phenyl]amino}propanoic Acid

Into a 50 mL round-bottom flask were added methyl 3-amino-4-chlorobenzoate (25 g, 130 mmol) and acrylic acid (200 mL) at rt. The resulting mixture was stirred for 4 h at 100° C. under nitrogen atmosphere. On completion, the mixture was cooled to rt and diluted with water (400 mL). The aqueous layer was extracted with EtOAc (3×100 mL). The combined organic layers were concentrated under reduced pressure to afford the title compound (25 g) as a yellow oil. LC/MS (ESI, m/z): [(M+1)]⁺=258.1.

### Step 2—Methyl 4-chloro-3-(2,4-dioxo-1,3-diazinan-1-yl)benzoate

To a stirred solution of 3-{[2-chloro-5-(methoxycarbonyl)phenyl]amino}propanoic acid (25 g, 97 mmol) in AcOH (200 mL) was added urea (40.79 g, 679.2 mmol) at rt under nitrogen atmosphere. The resulting mixture was then stirred for 16 h at 110° C. under nitrogen atmosphere. On completion, the mixture was cooled to rt and diluted with water (300 mL). The resulting mixture was extracted with EtOAc (3×100 mL). The combined organic layers were washed with brine (3×50 mL), and dried over anhydrous Na₂SO₄. After filtration, the filtrate was concentrated under reduced pressure. The resulting mixture was concentrated under reduced pressure. The residue was purified by trituration with MTBE (100 mL) to afford the title compound (14 g, 51% yield) as a white solid. LC/MS (ESI, m/z): [(M+1)]⁺=283.1.

### Step 3—4-Chloro-3-(2,4-dioxo-1,3-diazinan-1-yl)benzoic Acid

To a stirred solution of methyl 4-chloro-3-(2,4-dioxo-1,3-diazinan-1-yl)benzoate (8 g, 30 mmol) in THF (200 mL) was added trimethyl(potassiooxy)silane (5.45 g, 42.5 mmol) at rt under nitrogen atmosphere. The resulting mixture was stirred for 1 h at rt under nitrogen atmosphere. On completion, the mixture was acidified to pH 5 with formic acid. The precipitated solids were collected by filtration and washed with THF (3×10 mL). The collected solids was purified by trituration with diethyl ether (200 mL) to afford the title compound (6.4 g, 84% yield) as a white solid. LC/MS (ESI, m/z): [(M+1)]⁺=267.1.

### Example 1.291. Preparation of 6-{[3-(5-Formylpyridin-2-yl)phenyl]amino}-8-(methylamino)-N-(oxolan-3-yl)imidazo[1,2-b]pyridazine-3-carboxamide (Intermediate KU)

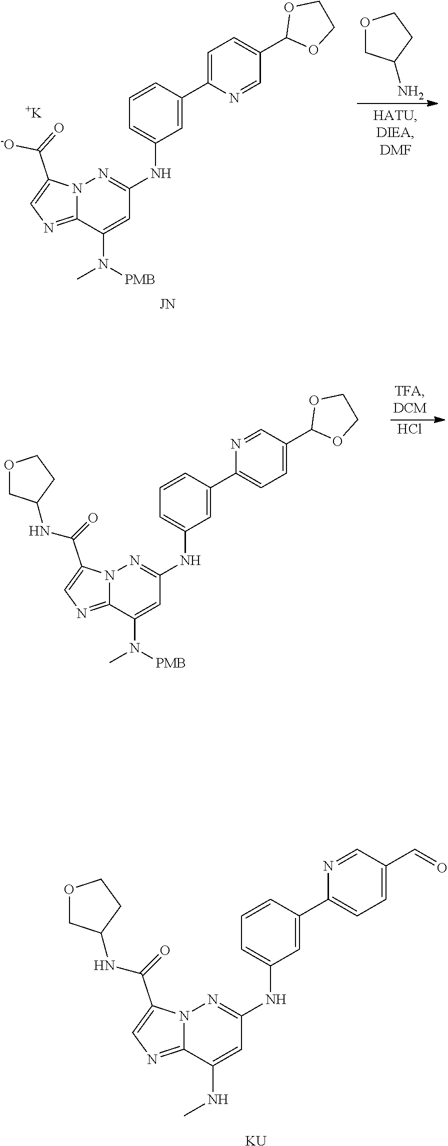

### Step 1—6-({3-[5-(1,3-Dioxolan-2-yl)pyridin-2-yl]phenyl}amino)-8-{[(4-methoxyphenyl)methyl](methyl)amino}-N-(oxolan-3-yl)imidazo[1,2-b]pyridazine-3-carboxamide

To a stirred solution of 6-({3-[5-(1,3-dioxolan-2-yl)pyridin-2-yl]phenyl}amino)-8-{[(4-methoxyphenyl)methyl](methyl)amino}imidazo[1,2-b]pyridazine-3-carboxylic acid potassium (400 mg, 0.7 mmol, Intermediate JN) and oxolan-3-amine (94.60 mg, 1.086 mmol, CAS #88675-24-5) in DMF (4 mL) were added HATU (412.86 mg, 1.086 mmol) and DIEA (0.25 mL, 1.5 mmol) in portions at rt under nitrogen atmosphere. The resulting mixture was stirred for 2 h at rt under nitrogen atmosphere. On completion, the residue was purified by trituration with water (20 mL). The precipitated solids were collected by filtration and washed with water (6 mL) to give the title compound (420 mg, 93% yield) as a white solid. LC/MS (ESI, m/z): [(M+H)]⁺=622.3.

### Step 2—6-{[3-(5-Formylpyridin-2-yl)phenyl]amino}-8-(methylamino)-N-(oxolan-3-yl)imidazo[1,2-b]pyridazine-3-carboxamide

To a stirred solution of 6-({3-[5-(1,3-dioxolan-2-yl)pyridin-2-yl]phenyl}amino)-8-{[(4-methoxyphenyl)methyl](methyl)amino}-N-(oxolan-3-yl)imidazo[1,2-b]pyridazine-3-carboxamide (300 mg, 0.5 mmol) in DCM (6 mL) were added TFA (3 mL) and 1M aq. HCl (3 mL) in portions at rt under nitrogen atmosphere. The resulting mixture was stirred for 16 h at 50° C. under nitrogen atmosphere. On completion, the mixture was concentrated under reduced pressure. The residue was purified by trituration with Et₂O (20 mL). The crude product was purified by Prep-HPLC (Column: Kinetex EVO C18 Column, 30*150, Sum; Mobile Phase A: Water (10 mmol/L NH₄HCO₃), Mobile Phase B: ACN; Flow rate: 60 mL/min; Gradient: 22% B to 32% B in 10 min, 32% B to 32% B in 12 min, 32% B; Wave Length: 254 nm; RT1 (min): 11.2) to afford the title compound (10.4 mg, 5% yield) as a white solid. LC/MS (ESI, m/z): [(M+H)]⁺=458.2.

### Example 1.292. N-[(1R,2S)-2-fluorocyclopropyl]-6-[4-(5-formylpyridin-2-yl)phenyl]-8-(methylamino)imidazo[1,2-b]pyridazine-3-carboxamide (Intermediate KV)

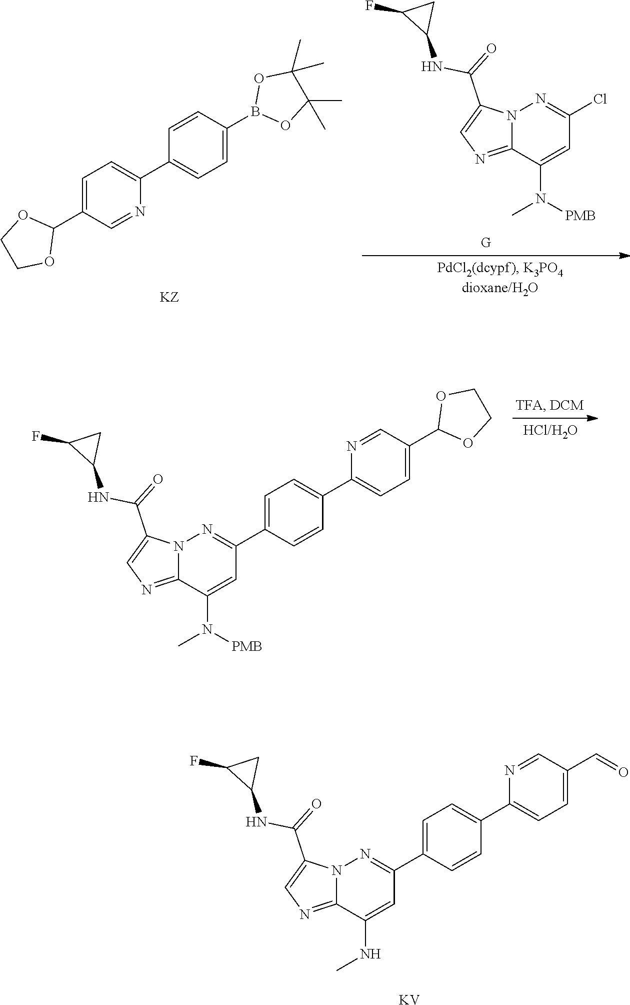

### Step 1—6-{4-[5-(1,3-Dioxolan-2-yl)pyridin-2-yl]phenyl}-N-[(1R,2S)-2-fluorocyclopropyl]-8-{[(4-methoxyphenyl)methyl](methyl)amino}imidazo[1,2-b]pyridazine-3-carboxamide

To a stirred solution of 5-(1,3-dioxolan-2-yl)-2-[4-(4,4,5,5-tetramethyl-1,3,2-dioxaborolan-2-yl)phenyl]pyridine (400 mg, 1 mmol, Intermediate KZ) and 6-chloro-N-((1R,2S)-2-fluorocyclopropyl)-8-((4-methoxybenzyl)(methyl)amino)imidazo[1,2-b]pyridazine-3-carboxamide (548.77 mg, 1.358 mmol, Intermediate G) in 1,4-dioxane (5 mL) and H₂O (1 mL) were added 1,1′-bis(di-tert-butylphosphino)ferrocene palladium dichloride PdCl₂(DTBPF) (73.80 mg, 0.113 mmol) and K₃PO₄ (480.74 mg, 2.264 mmol) in portions at rt under nitrogen atmosphere. The resulting mixture was stirred for 2 h at 90° C. under nitrogen atmosphere. On completion, the mixture was concentrated under reduced pressure. The residue was purified by silica gel column chromatography, eluted with PE/EA (1:1), to afford the title compound (380 mg, 56% yield) as a white solid. LC/MS (ESI, m/z): [(M+H)]⁺=595.2.

### Step 2—N-[(1R,2S)-2-fluorocyclopropyl]-6-[4-(5-formylpyridin-2-yl)phenyl]-8-(methylamino)imidazo[1,2-b]pyridazine-3-carboxamide

To a stirred solution of 6-{4-[5-(1,3-dioxolan-2-yl)pyridin-2-yl]phenyl}-N-[(1R,2S)-2-fluorocyclopropyl]-8-{[(4-methoxyphenyl)methyl](methyl)amino}imidazo[1,2-b]pyridazine-3-carboxamide (360 mg, 0.605 mmol) in DCM (6 mL) were added 1 M aq. HCl (3 mL) and TFA (3 mL) at rt under nitrogen atmosphere. The resulting mixture was stirred for 2 h at 50° C. under nitrogen atmosphere. On completion, the mixture was concentrated under reduced pressure. The crude product was purified by Prep-HPLC (Column: Xselect CSH F-Phenyl OBD column, 19*250 mm, 5 μm; Mobile Phase A: Water (0.1% FA), Mobile Phase B: ACN; Flow rate: 20 mL/min; Gradient: 57% B to 59% B in 8 min, 59% B; Wave Length: 220 nm; RT1 (min): 8) to afford the title compound (150 mg, 58% yield) as a white solid. LC/MS (ESI, m/z): [(M+H)]⁺=431.2.

### Example 1.293. Preparation of 3-(3-methyl-2-oxo-5-{4-[3-oxo-3-(piperazin-1-yl)propyl]phenyl}-1,3-benzodiazol-1-yl)piperidine-2,6-dione (Intermediate KW)

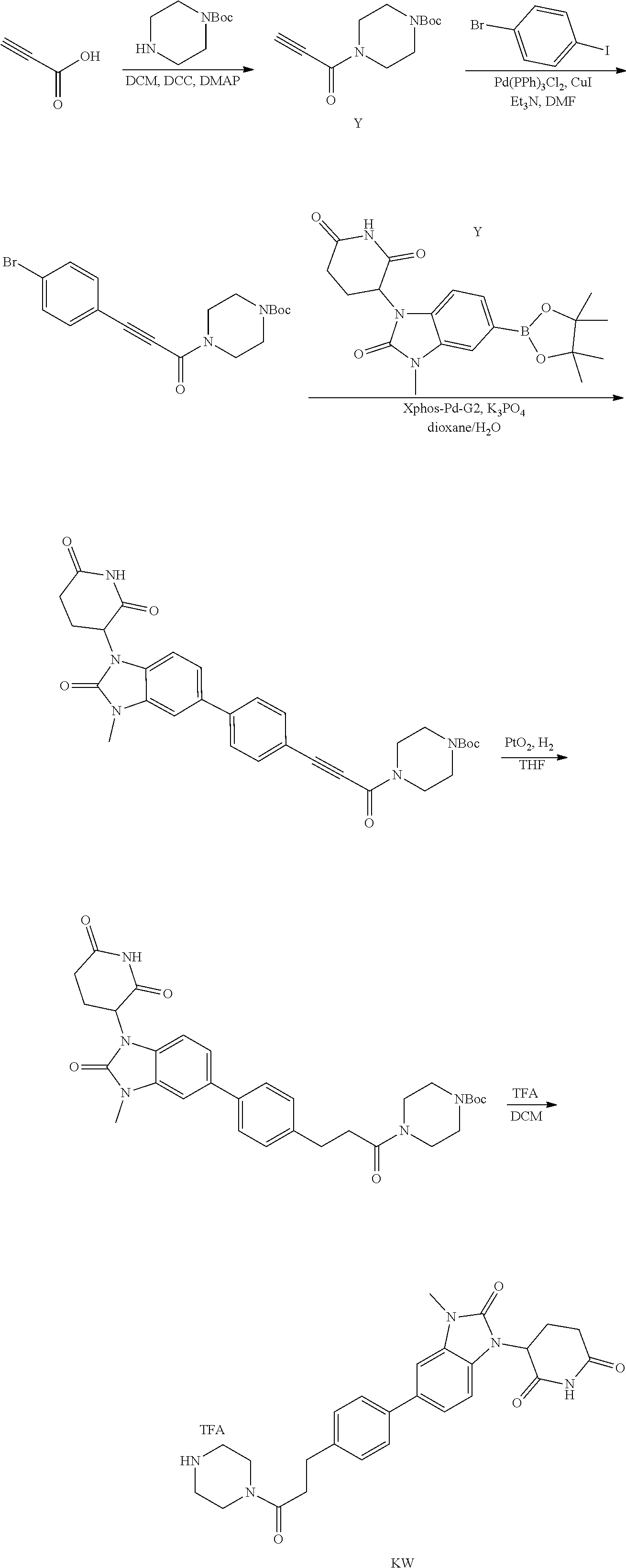

### Step 1—Tert-butyl 4-propioloylpiperazine-1-carboxylate

To a solution of propiolic acid (4 g, 60 mmol) and tert-butyl piperazine-1-carboxylate (10.7 g, 57.7 mmol) in dichloromethane (100 mL) at 0° C. was added dicyclohexylmethanediimine (11.8 g, 57.1 mmol) and N,N-dimethylpyridin-4-amine (698 mg, 5.71 mmol), then the reaction mixture was stirred at 25° C. for 12 hrs. On completion, the reaction mixture was filtered and concentrated under reduced pressure to give a residue. The residue was purified by column chromatography (SiO₂, Petroleum ether/Ethyl acetate=20/1 to 1/1) to give the title compound (8 g, 58% yield) as white solid. ¹H NMR (400 MHz, CDCl₃-d) δ=3.80-3.73 (m, 2H), 3.66-3.60 (m, 2H), 3.54-3.48 (m, 2H), 3.47-3.41 (m, 2H), 3.18 (s, 1H), 1.49 (s, 9H).

### Step 2—Tert-butyl 4-(3-(4-bromophenyl)propioloyl)piperazine-1-carboxylate

A mixture of 1-bromo-4-iodobenzene (3 g, 10 mmol), tert-butyl 4-propioloylpiperazine-1-carboxylate (2.78 g, 11.7 mmol), bis(triphenylphosphine)palladium(II)dichloride (744 mg, 1.06 mmol), copper iodide (404 mg, 2.12 mmol) and triethylamine (3.22 g, 31.8 mmol) in dimethylformamide (60 mL) was degassed and purged with nitrogen for 3 times. Then the mixture was stirred at 25° C. for 1 hr under nitrogen atmosphere. The reaction mixture was diluted with water (50 mL) and extracted with ethyl acetate (80 mL×2). The combined organic layers were washed with brine (100 mL×2), dried over anhydrous sodium sulfate, filtered and concentrated under reduced pressure to give a residue. The residue was purified by column chromatography (SiO₂, Petroleum ether/Ethyl acetate=50/1 to 0/1) to give the title compound (2.5 g, 55% yield) as yellow solid. LC-MS (ESI+) m/z 416.8 (M+Na)⁺.

### Step 3—Tert-butyl 4-(3-(4-(1-(2,6-dioxopiperidin-3-yl)-3-methyl-2-oxo-2,3-dihydro-1H-benzo[d]imidazol-5-yl)phenyl)propioloyl)piperazine-1-carboxylate

A mixture of tert-butyl 4-[3-(4-bromophenyl)prop-2-ynoyl]piperazine-1-carboxylate (2.2 g, 5.6 mmol), 3-[3-methyl-2-oxo-5-(4,4,5,5-tetramethyl-1,3,2-dioxaborolan-2-yl)benzimidazol-1-yl]piperidine-2,6-dione (2.15 g, 5.59 mmol, Intermediate Y), chloro(2-dicyclohexylphosphino-2,4,6-triisopropyl-1,1-biphenyl)[2-(2-amino-1,1-biphenyl)]palladium(II) (440 mg, 560 umol) and potassium phosphate (2.37 g, 11.2 mmol) in dioxane (44 mL) and water (4 mL) was degassed and purged with nitrogen for 3 times. Then the mixture was stirred at 80° C. for 12 hrs under nitrogen atmosphere. The reaction mixture was diluted with water (30 mL) and extracted with ethyl acetate (40 mL×2). The combined organic layers were washed with brine (50 mL×2), dried over anhydrous sodium sulfate, filtered and concentrated under reduced pressure to give the title compound (2.1 g) as a yellow solid. LC-MS (ESI+) m/z 572.1 (M+H)⁺.

### Step 4—Tert-butyl 4-(3-(4-(1-(2,6-dioxopiperidin-3-yl)-3-methyl-2-oxo-2,3-dihydro-1H-benzo[d]imidazol-5-yl)phenyl)propanoyl)piperazine-1-carboxylate

To a solution of tert-butyl 4-[3-[4-[1-(2,6-dioxo-3-piperidyl)-3-methyl-2-oxo-benzimidazol-5-yl]phenyl]prop-2-ynoyl]piperazine-1-carboxylate (1.8 g, 3.2 mmol) in tetrahydrofuran (40 mL) was added PtO2 (715 mg, 3.15 mmol) under nitrogen atmosphere. The suspension was degassed under vacuum and purged with H₂ several times. The mixture was stirred under hydrogen (15 psi) at 25° C. for 4 hrs. On completion, the reaction mixture was filtered and concentrated under reduced pressure to give a residue. The residue was purified by prep-HPLC (column: Phenomenex luna C18 150*40 mm*15 um; mobile phase: [water (FA)-ACN]; B %: 32%-62%, 10 min) to give the title compound (0.77 g, 24% yield for two steps) as a yellow solid. ¹H NMR (400 MHz, DMSO-d₆) δ=11.12 (s, 1H), 7.60 (d, J=8.0 Hz, 2H), 7.47 (s, 1H), 7.33 (d, J=8.0 Hz, 3H), 7.18 (d, J=8.4 Hz, 1H), 5.43-5.38 (m, 1H), 3.46-3.40 (m, 6H), 3.30-3.24 (M, 4H), 2.98-2.82 (m, 3H), 2.80-2.60 (m, 5H), 2.08-2.04 (m, 1H), 1.40 (s, 9H); LC-MS (ESI⁺) m/z 598.1 (M+Na)⁺.

### Step 5—3-(3-methyl-2-oxo-5-{4-[3-oxo-3-(piperazin-1-yl)propyl]phenyl}-1,3-benzodiazol-1-yl)piperidine-2,6-dione trifluoroacetate

To a stirred solution of tert-butyl 4-(3-{4-[1-(2,6-dioxopiperidin-3-yl)-3-methyl-2-oxo-1,3-benzodiazol-5-yl]phenyl}propanoyl)piperazine-1-carboxylate (75 mg, 0.13 mmol) in DCM (2 mL) was added TFA (1 mL) dropwise at rt under nitrogen atmosphere. The resulting mixture was stirred for 2 h at rt under nitrogen atmosphere. On completion, the mixture was concentrated under reduced pressure. The residue was purified by trituration with Et₂O (3 mL) to afford the title compound (70 mg, 94% yield) as a white solid. LC/MS (ESI, m/z): [(M+H)]⁺=476.3.

### Example 1.294. Preparation of 3-[3-methyl-2-oxo-5-(4-oxopiperidin-1-yl)-1,3-benzodiazol-1-yl]piperidine-2,6-dione (Intermediate KX)

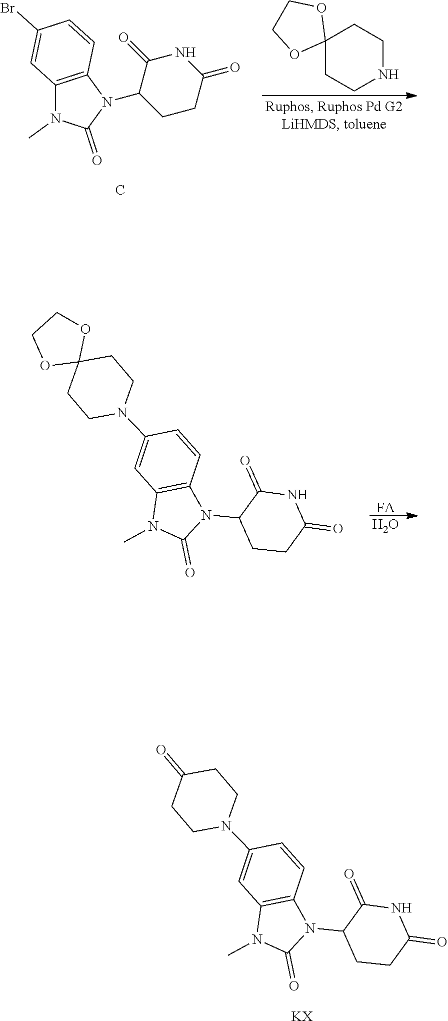

### Step 1—3-(5-{1,4-Dioxa-8-azaspiro[4.5]decan-8-yl}-3-methyl-2-oxo-1,3-benzodiazol-1-yl)piperidine-2,6-dione

To a stirred solution of 3-(5-bromo-3-methyl-2-oxo-1,3-benzodiazol-1-yl)piperidine-2,6-dione (5 g, 10 mmol, Intermediate C) and 1,4-dioxa-8-azaspiro[4.5]decane (3.18 g, 22.2 mmol, CAS #177-11-7) in toluene (100 mL) was added LiHMDS (14.84 g, 88.72 mmol) at 0° C. under argon atmosphere. To the above mixture was added RuPhos-PdCl-2nd G (1.15 g, 1.48 mmol) and RuPhos (1.38 g, 3.00 mmol) at rt. The resulting mixture was stirred for additional 2 h at 80° C. On completion, the mixture was cooled to rt and acidified to pH 4 with formic acid. The resulting mixture was concentrated under reduced pressure. The residue was purified by silica gel column chromatography, eluted with CH₂Cl₂/MeOH (10:1), to afford the title compound (5 g, 85% yield) as a green solid. LC/MS (ESI, m/z): [(M+H)]⁺=401.2.

### Step 2—3-[3-Methyl-2-oxo-5-(4-oxopiperidin-1-yl)-1,3-benzodiazol-1-yl]piperidine-2,6-dione

To a stirred solution of 3-(5-{1,4-dioxa-8-azaspiro[4.5]decan-8-yl}-3-methyl-2-oxo-1,3-benzodiazol-1-yl)piperidine-2,6-dione (2 g, 5 mmol) were added formic acid (15 mL) and H₂O (3 mL) dropwise at rt under nitrogen atmosphere. The resulting mixture was stirred for 16 h at 50° C. under nitrogen atmosphere. On completion, the mixture was cooled to rt and concentrated under reduced pressure. The residue was purified by reverse phase flash chromatography (Column: WelFlash™ C18-I, 20-40 um, 330 g; Eluent A: Water (plus 10 mmol/L NH₄HCO₃); Eluent B: ACN; Gradient: 20%-55% B in 25 min; Flow rate: 80 mL/min; Detector: 220/254 nm; desired fractions were collected at 30% B) to afford the title compound (730 mg, 41% yield) as a light orange solid. LC/MS (ESI, m/z): [(M+H)]⁺=357.1.

### Example 1.295. Preparation of 6-{[2-chloro-3-(5-formylpyridin-2-yl)phenyl]amino}-N-[(1R,2S)-2-fluorocyclopropyl]-8-(methylamino)imidazo[1,2-b]pyridazine-3-carboxamide (Intermediate KY)

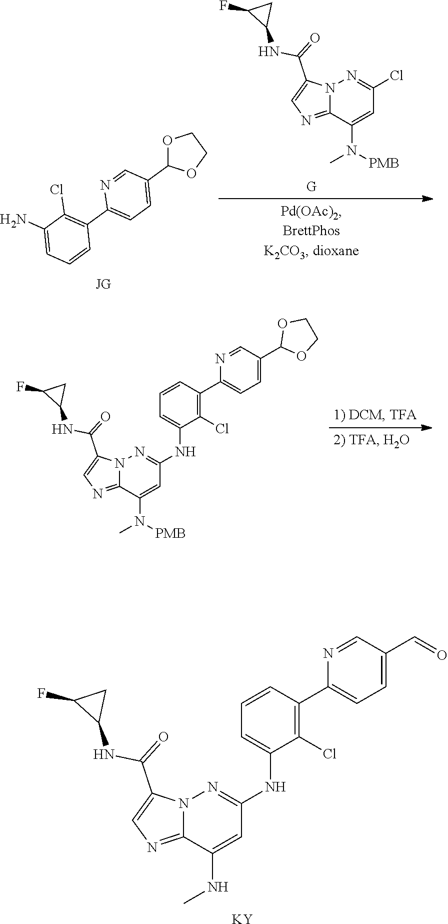

### Step 1—6-({2-chloro-3-[5-(1,3-dioxolan-2-yl)pyridin-2-yl]phenyl}amino)-N-[(1R,2S)-2-fluorocyclopropyl]-8-{[(4-methoxyphenyl)methyl](methyl)amino}imidazo[1,2-b]pyridazine-3-carboxamide

To a stirred mixture of 2-chloro-3-[5-(1,3-dioxolan-2-yl)pyridin-2-yl]aniline (1 g, 4 mmol, Intermediate JG) and 6-chloro-N-[(1R,2S)-2-fluorocyclopropyl]-8-{[(4-methoxyphenyl)methyl](methyl)amino}imidazo[1,2-b]pyridazine-3-carboxamide (1.75 g, 4.34 mmol, Intermediate G) in dioxane (10 mL) were added Pd(OAc)₂ (0.08 g, 0.4 mmol) and BrettPhos (0.19 g, 0.36 mmol), K₂CO₃ (1.50 g, 10.9 mmol) at rt under nitrogen atmosphere. The resulting mixture was stirred for 4 h at 100° C. under nitrogen atmosphere. On completion, the mixture was cooled to rt and concentrated under reduced pressure. The residue was purified by reverse phase Flash chromatography (Column: WelFlash™ C18-I, 20-40 um, 330 g; Eluent A: Water (plus 10 mmol/L FA); Eluent B: ACN; Gradient: 25%-70% B in 20 min; Flow rate: 85 mL/min; Detector: 220/254 nm; desired fractions were collected at 55% B) to afford the title compound (500 mg, 22% yield) as a light brown solid. LC/MS (ESI, m/z): [(M+H)]⁺=644.2.

### Step 2—6-{[2-chloro-3-(5-formylpyridin-2-yl)phenyl]amino}-N-[(1R,2S)-2-fluorocyclopropyl]-8-(methylamino)imidazo[1,2-b]pyridazine-3-carboxamide

To a stirred mixture of 6-({2-chloro-3-[5-(1,3-dioxolan-2-yl)pyridin-2-yl]phenyl}amino)-N-[(1R,2S)-2-fluorocyclopropyl]-8-{[(4-methoxyphenyl)methyl](methyl)amino}imidazo[1,2-b]pyridazine-3-carboxamide (400 mg, 0.6 mmol) in DCM (5 mL) was added TFA (3 mL) at rt under nitrogen atmosphere. The resulting mixture was stirred for 4 h at rt under nitrogen atmosphere. The resulting mixture was concentrated under reduced pressure. To the above residue was added H₂O (5 mL) at rt. The resulting mixture was stirred for additional 2 h at 60° C. On completion, the mixture was concentrated under reduced pressure. The residue was purified by reverse phase Flash chromatography (Column: WelFlash™ C18-I, 20-40 um, 330 g; Eluent A: Water (plus 10 mmol/L FA); Eluent B: ACN; Gradient: 20%-65% B in 20 min; Flow rate: 90 mL/min; Detector: 220/254 nm; desired fractions were collected at 50% B) to afford the title compound (150 mg, 50% yield) as a light pink solid. LC/MS (ESI, m/z): [(M+H)]⁺=480.2.

### Example 1.296. Preparation of (1,3-dioxolan-2-yl)-2-[4-(4,4,5,5-tetramethyl-1,3,2-dioxaborolan-2-yl)phenyl]pyridine (Intermediate KZ)

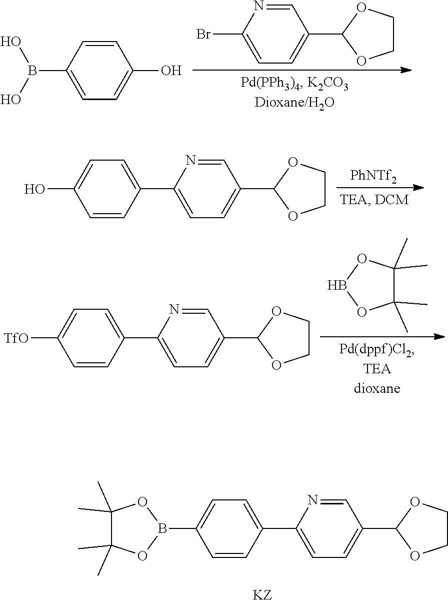

### Step 1—4-[5-(1,3-Dioxolan-2-yl)pyridin-2-yl]phenol

To a stirred solution of 4-hydroxyphenylboronic acid (7.91 g, 57.4 mmol) and 2-bromo-5-(1,3-dioxolan-2-yl)pyridine (11 g, 48 mmol, synthesized via Step 1 of Intermediate BC) in dioxane (100 mL) and H₂O (20 mL) was added K₂CO₃ (19.82 g, 143.4 mmol) at rt under nitrogen atmosphere. To the above mixture was added Pd(PPh₃)₄ (5.53 g, 4.78 mmol) at rt. The resulting mixture was stirred for additional 5 min at rt. The resulting mixture was stirred for 16 h at 80° C. under nitrogen atmosphere. On completion, the mixture was cooled to rt and concentrated under reduced pressure. The residue was purified by silica gel column chromatography, eluted with PE/EA (5:1), to afford the title compound (3.9 g, 34% yield) as a light brown solid. LC/MS (ESI, m/z): [(M+H)]⁺=244.1.

### Step 2—4-[5-(1,3-Dioxolan-2-yl)pyridin-2-yl]phenyl Trifluoromethanesulfonate

To a stirred solution of 4-[5-(1,3-dioxolan-2-yl)pyridin-2-yl]phenol (3.842 g, 15.79 mmol) and 1,1,1-trifluoro-N-phenyl-N-trifluoromethanesulfonylmethanesulfonamide (8.46 g, 23.7 mmol) in DCM (30 mL) were added TEA (6.42 mL, 47.4 mmol) and DMAP (0.19 g, 1.6 mmol) rt under nitrogen atmosphere. The resulting mixture was stirred for 1 h at rt under nitrogen atmosphere. On completion, the mixture was concentrated under reduced pressure. The residue was purified by silica gel column chromatography, eluted with PE/EA (1:1), to afford the title compound (5 g, 84% yield) as a white solid. LC/MS (ESI, m/z): [(M+H)]⁺=376.1.

### Step 3—(1,3-Dioxolan-2-yl)-2-[4-(4,4,5,5-tetramethyl-1,3,2-dioxaborolan-2-yl)phenyl]pyridine

To a stirred solution of 4-[5-(1,3-dioxolan-2-yl)pyridin-2-yl]phenyl trifluoromethanesulfonate (2.4 g, 6.395 mmol) and 4,4,5,5-tetramethyl-1,3,2-dioxaborolane (5.32 g, 41.6 mmol) in dioxane (30 mL) was added TEA (6.12 mL, 44.8 mmol) at rt under nitrogen atmosphere. To the above mixture was added Pd(dppf)Cl₂CH₂Cl₂ (0.52 g, 0.64 mmol) at rt under nitrogen atmosphere. The mixture was stirred for additional 5 min at rt. The mixture was then stirred for 2 h at 100° C. under nitrogen atmosphere. On completion, the mixture was cooled to rt and concentrated under reduced pressure. The residue was purified by silica gel column chromatography, eluted with PE/EA (7:1), to afford the title compound (2 g, 89% yield) as an off-white solid. LC/MS (ESI, m/z): [(M+H)]⁺=354.2.

### Example 1.297. Preparation of N-((1R,5S,6s)-3-oxabicyclo[3.1.0]hexan-6-yl)-6-((3-(5-formylpyridin-2-yl)phenyl)amino)-8-(methylamino)imidazo[1,2-b]pyridazine-3-carboxamide (Intermediate LA)

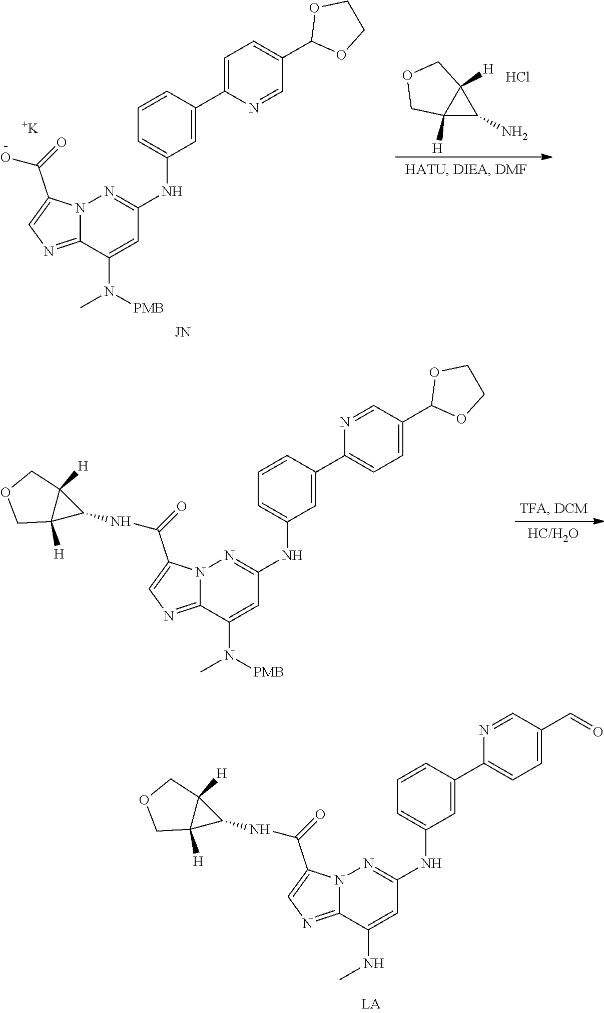

### Step 1—6-((3-(5-(1,3-Dioxolan-2-yl)pyridin-2-yl)phenyl)amino)-N-((1R,5S,6s)-3-oxabicyclo[3.1.0]hexan-6-yl)-8-((4-methoxybenzyl)(methyl)amino)imidazo[1,2-b]pyridazine-3-carboxamide

To a stirred solution of (1R,5S,6R)-3-oxabicyclo[3.1.0]hexan-6-amine hydrochloride (184.03 mg, 1.357 mmol, CAS #1285720-68-4) and potassium 6-((3-(5-(1,3-dioxolan-2-yl)pyridin-2-yl)phenyl)amino)-8-((4-methoxybenzyl)(methyl)amino)imidazo[1,2-b]pyridazine-3-carboxylate (500 mg, 0.9 mmol, Intermediate JN) in DMF (6 mL) were added HATU (516.07 mg, 1.357 mmol) and DIEA (0.32 mL, 1.810 mmol) at rt temperature under nitrogen atmosphere. The resulting mixture was stirred for 2 h at rt under nitrogen atmosphere. On completion, the mixture was poured into water (20 mL). The precipitated solids were collected by filtration, the filter cake was washed with ACN (10 mL) to afford the title compound (400 mg, 70% yield) as an off-white solid. LC/MS (ESI, m/z): [(M+H)]⁺=634.2.

### Step 2—N-((1R,5S,6s)-3-oxabicyclo[3.1.0]hexan-6-yl)-6-((3-(5-formylpyridin-2-yl)phenyl)amino)-8-(methylamino)imidazo[1,2-b]pyridazine-3-carboxamide

To a stirred solution of 6-((3-(5-(1,3-dioxolan-2-yl)pyridin-2-yl)phenyl)amino)-N-((1R,5S,6s)-3-oxabicyclo[3.1.0]hexan-6-yl)-8-((4-methoxybenzyl)(methyl)amino)imidazo[1,2-b]pyridazine-3-carboxamide (400 mg, 0.6 mmol) in DCM (4 mL) were added TFA (4 mL) and 1 M aq. HCl (4 mL) in portions at rt under nitrogen atmosphere. The resulting mixture was stirred for 16 h at 50° C. under nitrogen atmosphere. On completion, the mixture was concentrated under reduced pressure. The crude product was purified by Prep-HPLC (Column: Kinetex EVO C18 Column, 30*150, Sum; Mobile Phase A: Water (10 mmol/L NH₄HCO₃), Mobile Phase B: ACN; Flow rate: 60 mL/min; Gradient: 22% B to 32% B in 10 min, 32% B; Wave Length: 254 nm; RT1 (min): 11.2 to afford the title compound (200 mg, 67% yield) as a white solid. LC/MS (ESI, m/z): [(M+H)]⁺=470.2.

### Example 1.298. Preparation of N-(1-cyano-1-methylethyl)-6-{[3-(5-formylpyridin-2-yl)phenyl]amino}-8-(methylamino)imidazo[1,2-b]pyridazine-3-carboxamide (Intermediate LB)

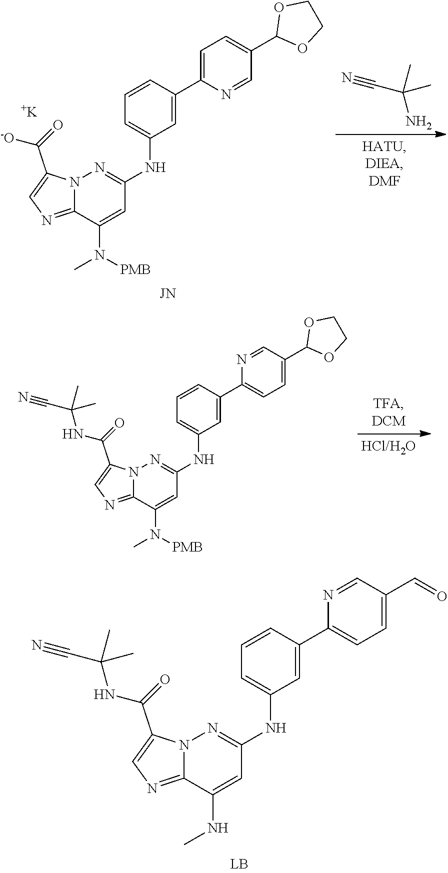

### Step 1—N-(1-cyano-1-methylethyl)-6-({3-[5-(1,3-dioxolan-2-yl)pyridin-2-yl]phenyl}amino)-8-{[(4-methoxyphenyl)methyl](methyl)amino}imidazo[1,2-b]pyridazine-3-carboxamide

To a stirred solution of potassium 6-((3-(5-(1,3-dioxolan-2-yl)pyridin-2-yl)phenyl)amino)-8-((4-methoxybenzyl)(methyl)amino)imidazo[1,2-b]pyridazine-3-carboxylate (400 mg, 0.7 mmol, Intermediate JN) and 2-amino-2-methylpropanenitrile (68.24 mg, 0.811 mmol, CAS #19355-69-2) in DMF (6 mL) were added HATU (385.58 mg, 1.014 mmol) and DIEA (0.35 mL, 2.0 mmol) in portions at rt under nitrogen atmosphere. The resulting mixture was stirred for 2 h at rt under nitrogen atmosphere. On completion, the mixture was poured into water (20 mL). The precipitated solids were collected by filtration, the filter cake was washed with ACN (10 mL) to afford the title compound (202 mg, 48% yield) as a yellow solid. LC/MS (ESI, m/z): [(M+H)]⁺=619.3.

### Step 2—N-(1-cyano-1-methylethyl)-6-{[3-(5-formylpyridin-2-yl)phenyl]amino}-8-(methylamino)imidazo[1,2-b]pyridazine-3-carboxamide

To a stirred solution of N-(1-cyano-1-methylethyl)-6-({3-[5-(1,3-dioxolan-2-yl)pyridin-2-yl]phenyl}amino)-8-{[(4-methoxyphenyl)methyl](methyl)amino}imidazo[1,2-b]pyridazine-3-carboxamide (120 mg, 0.19 mmol) in DCM (3 mL) were added TFA (1 mL) and 1 M aq. HCl (1 mL) in portions at rt under nitrogen atmosphere. The resulting mixture was stirred for 16 h at 50° C. under nitrogen atmosphere. On completion, the mixture was concentrated under reduced pressure. The residue was purified by reverse phase Flash chromatography (Column: WelFlash™ C18-I, 20-40 um, 120 g; Eluent A: Water (plus 10 mmol/L FA); Eluent B: ACN; Gradient: 25%-55% B in 25 min; Flow rate: 80 mL/min; Detector: 220/254 nm; desired fractions were collected at 40% B) to afford the title compound (19.5 mg, 20% yield) as a light yellow solid. LC/MS (ESI, m/z): [(M+H)]⁺=455.2.

### Example 1.299. Preparation of 6-{[3-(5-formylpyridin-2-yl)phenyl]amino}-N-[(1R)-2-methoxycyclopropyl]-8-(methylamino)imidazo[1,2-b]pyridazine-3-carboxamide (Intermediate LC)

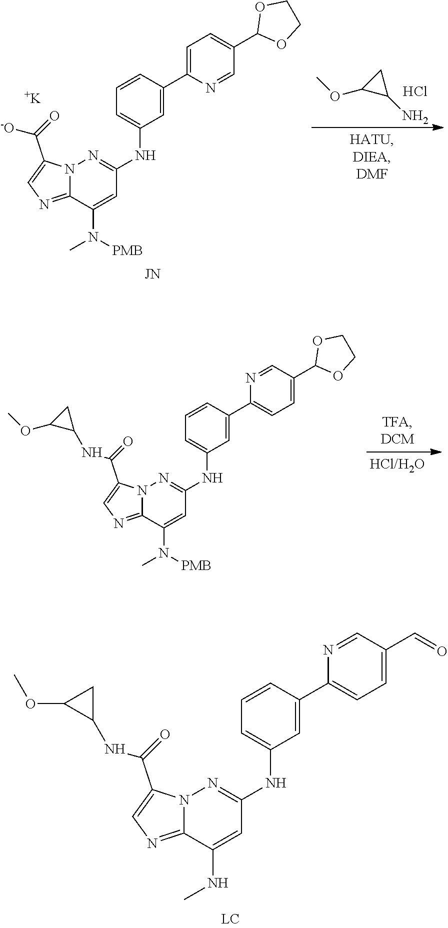

### Step 1—6-({3-[5-(1,3-Dioxolan-2-yl)pyridin-2-yl]phenyl}amino)-N-(2-methoxycyclopropyl)-8-{[(4-methoxyphenyl)methyl](methyl)amino}imidazo[1,2-b]pyridazine-3-carboxamide

To a stirred solution of potassium 6-((3-(5-(1,3-dioxolan-2-yl)pyridin-2-yl)phenyl)amino)-8-((4-methoxybenzyl)(methyl)amino)imidazo[1,2-b]pyridazine-3-carboxylate (400 mg, 0.6 mmol, Intermediate JN) and 2-methoxycyclopropan-1-amine hydrochloride (14.91 mg, 0.120 mmol, CAS #1046469-17-3) in DMF (6 mL) were added HATU (366.95 mg, 0.965 mmol) and DIEA (0.22 mL, 1.3 mmol) in portions at rt under nitrogen atmosphere. The resulting mixture was stirred for 2 h at rt under nitrogen atmosphere. On completion, the mixture was poured into water (20 mL). The precipitated solids were collected by filtration, and the filter cake was washed with ACN (10 mL) to afford the title compound (300 mg, 75% yield) as a white solid. LC/MS (ESI, m/z): [(M+H)]⁺=622.3.

### Step 2—6-{[3-(5-formylpyridin-2-yl)phenyl]amino}-N-[(1R)-2-methoxycyclopropyl]-8-(methylamino)imidazo[1,2-b]pyridazine-3-carboxamide

To a stirred solution of 6-({3-[5-(1,3-dioxolan-2-yl)pyridin-2-yl]phenyl}amino)-N-[(1R)-2-methoxycyclopropyl]-8-{[(4-methoxyphenyl)methyl](methyl)amino}imidazo[1,2-b]pyridazine-3-carboxamide (100 mg, 0.2 mmol) in DCM (2 mL) were added TFA (2 mL) and 1 M aq. HCl (2 mL) in portions at rt under nitrogen atmosphere. The resulting mixture was stirred for 16 h at 50° C. under nitrogen atmosphere. On completion, the mixture was concentrated under reduced pressure. The crude product was purified by Prep-HPLC (Column: Kinetex EVO C18 Column, 30*150, Sum; Mobile Phase A: Water (10 mmol/L NH₄HCO₃), Mobile Phase B: ACN; Flow rate: 60 mL/min; Gradient: 27% B to 37% B in 10 min, 37% B; Wave Length: 254 nm; RT1 (min): 11.3) to afford the title compound (5.8 mg, 8% yield) as a yellow solid. LC/MS (ESI, m/z): [(M+H)]⁺=458.2.

### Example 1.300. Preparation of N-cyclobutyl-6-{[3-(5-formylpyridin-2-yl)phenyl]amino}-8-(methylamino)imidazo[1,2-b]pyridazine-3-carboxamide (Intermediate LD)

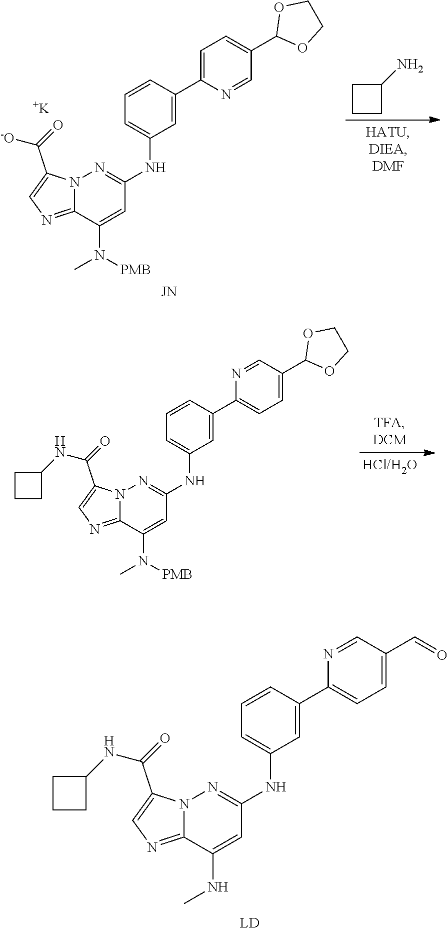

### Step 1—N-cyclobutyl-6-({3-[5-(1,3-dioxolan-2-yl)pyridin-2-yl]phenyl}amino)-8-{[(4-methoxyphenyl)methyl](methyl)amino}imidazo[1,2-b]pyridazine-3-carboxamide

To a stirred solution of 6-({3-[5-(1,3-dioxolan-2-yl)pyridin-2-yl]-2-methoxyphenyl}amino)-8-{[(4-methoxyphenyl)methyl](methyl)amino}imidazo[1,2-b]pyridazine-3-carboxylic acid potassium (400 mg, 0.6 mmol, Intermediate JN) and cyclobutylamine (68.64 mg, 0.965 mmol) in DMF (6 mL) were added HATU (366.95 mg, 0.965 mmol) and DIEA (0.22 mL, 1.286 mmol) in portions at rt under nitrogen atmosphere. The resulting mixture was stirred for 2 h at rt under nitrogen atmosphere. On completion, the mixture was filtered, and the filter cake was washed with MeCN (10 mL). The filter cake was dried under reduced pressure to afford the title compound (350 mg, 90% yield) as a white solid. LC/MS (ESI, m/z): [(M+H)]⁺=606.3.

### Step 2—N-cyclobutyl-6-{[3-(5-formylpyridin-2-yl)phenyl]amino}-8-(methylamino)imidazo[1,2-b]pyridazine-3-carboxamide

To a stirred solution of N-cyclobutyl-6-({3-[5-(1,3-dioxolan-2-yl)pyridin-2-yl]phenyl}amino)-8-{[(4-methoxyphenyl)methyl](methyl)amino}imidazo[1,2-b]pyridazine-3-carboxamide (350 mg, 0.58 mmol) in DCM (4 mL) were added TFA (2 mL) and HCl (2 mL) in portions at rt under nitrogen atmosphere. The resulting mixture was stirred for 16 h at 50° C. under nitrogen atmosphere. On completion, the mixture was concentrated under reduced pressure. The residue was triturated with Et₂O to afford the title compound (200 mg) as a white solid. LC/MS (ESI, m/z): [(M+H)]⁺=442.2.

### Example 1.301. Preparation of N-[(1R,2S)-2-fluorocyclopropyl]-6-({2-methoxy-3-[4-(methoxymethylidene)cyclohexyl]phenyl}amino)-8-(methylamino)imidazo[1,2-b]pyridazine-3-carboxamide (Intermediate LE)

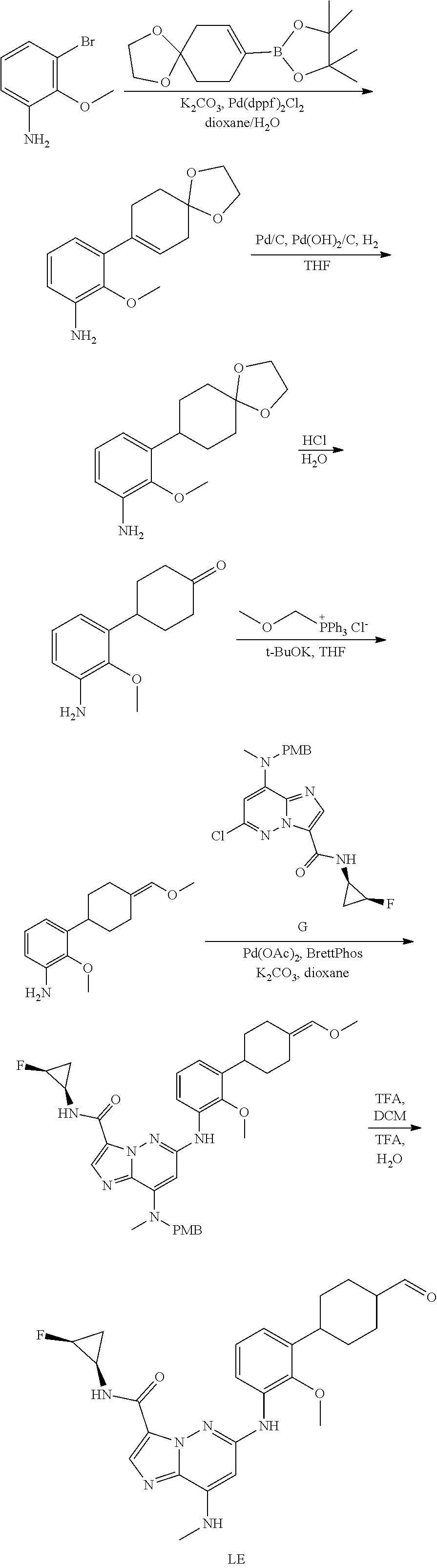

### Step 1—3-{1,4-Dioxaspiro[4.5]dec-7-en-8-yl}-2-methoxyaniline

To a stirred mixture of 3-bromo-2-methoxyaniline (10 g, 50 mmol) and 2-{1,4-dioxaspiro[4.5]dec-7-en-8-yl}-4,4,5,5-tetramethyl-1,3,2-dioxaborolane (13.17 g, 49.49 mmol) in dioxane (50 mL) and H₂O (10 mL) were added K₂CO₃ (13.68 g, 98.98 mmol) and Pd(dppf)Cl₂ (3.62 g, 4.95 mmol) at rt. The resulting mixture was stirred for 2 h at 80° C. under nitrogen atmosphere. On completion, the mixture was cooled to rt and concentrated under reduced pressure. The residue was purified by silica gel column chromatography, eluted with PE/EA (5:1), to afford the title compound (11 g, 85% yield) as a brown solid. LC/MS (ESI, m/z): [(M+H)]⁺=262.3.

### Step 2—3-{1,4-Dioxaspiro[4.5]decan-8-yl}-2-methoxyaniline

To a solution of 3-{1,4-dioxaspiro[4.5]dec-7-en-8-yl}-2-methoxyaniline (11 g, 42 mmol) in THF (110 mL) were added Pd/C (1 g, 10 mmol, 10 wt %) and Pd(OH)₂/C (1 g, 7 mmol) under nitrogen atmosphere. The reaction system was degassed under vacuum and purged with H₂ several times. Then the mixture was hydrogenated under H₂ balloon (˜1 atm) at 25° C. for 2 h. After completion of the reaction, Pd/C and Pd(OH)₂/C were filtered off through celite. The filter cake was washed with THF (3×10 mL). The corresponding filtrate was concentrated under reduced pressure to afford the title compound (8 g, 61% yield) as a brown solid. LC/MS (ESI, m/z): [(M+H)]⁺=264.3.

### Step 3—4-(3-Amino-2-methoxyphenyl)cyclohexan-1-one

To a stirred mixture of 3-{1,4-dioxaspiro[4.5]decan-8-yl}-2-methoxyaniline (2 g, 8 mmol) in H₂O (40 mL) was added a solution of HCl (20 mL) at rt. The resulting mixture was stirred for 6 h at 40° C. On completion, the mixture was cooled to rt and basified to pH 8 with NaOH. The resulting mixture was extracted with Et₂O (3×10 mL). The combined organic layers were washed with water (3×7 mL), and dried over anhydrous Na₂SO₄. After filtration, the filtrate was concentrated under reduced pressure to afford the title compound (1.19 g, 72% yield) as a brown oil. LC/MS (ESI, m/z): [(M+H)]⁺=220.2.

### Step 4—2-Methoxy-3-[4-(methoxymethylidene)cyclohexyl]aniline

To a stirred mixture of (methoxymethyl)triphenylphosphanium chloride (3.75 g, 10.9 mmol) in THF (10 mL) was added t-BuOK (1.84 g, 16.4 mmol) at 0° C. The resulting mixture was stirred for 20 min at 0° C. under nitrogen atmosphere. To the above mixture was added 4-(3-amino-2-methoxyphenyl)cyclohexan-1-one (1.19 g) at rt. The resulting mixture was stirred for additional 4 h at rt. On completion, the mixture was concentrated under reduced pressure. The residue was purified by reverse phase Flash chromatography (Column: WelFlash™ C18-I, 20-40 um, 120 g; Eluent A: Water (plus 10 mmol/L FA); Eluent B: ACN; Gradient: 25%-55% B in 25 min; Flow rate: 60 mL/min; Detector: 254 nm; desired fractions were collected at 50% B) to afford the title compound (0.8 mg, 59% yield) as a white solid. LC/MS (ESI, m/z): [(M+H)]⁺=248.3.

### Step 5—N-[(1R,2S)-2-fluorocyclopropyl]-6-({2-methoxy-3-[4-(methoxymethylidene)cyclohexyl]phenyl}amino)-8-{[(4-methoxyphenyl)methyl](methyl)amino}imidazo[1,2-b]pyridazine-3-carboxamide

To a stirred mixture of 2-methoxy-3-[4-(methoxymethylidene)cyclohexyl]aniline (422 mg, 1.75 mmol) and 6-chloro-N-[(1R,2S)-2-fluorocyclopropyl]-8-{[(4-methoxyphenyl)methyl](methyl)amino}imidazo[1,2-b]pyridazine-3-carboxamide (1 g, 3 mmol, Intermediate G) in dioxane (5 mL) were added Pd(OAc)₂ (39.30 mg, 0.175 mmol) and K₂CO₃ (483.98 mg, 3.502 mmol) at rt under nitrogen atmosphere. The resulting mixture was stirred for an additional 16 h at 80° C. under nitrogen atmosphere. On completion, the mixture was cooled to rt and concentrated under reduced pressure. The residue was purified by reverse phase Flash chromatography (Column: WelFlash™ C18-I, 20-40 um, 120 g; Eluent A: Water (plus 10 mmol/L NH₄HCO₃); Eluent B: ACN; Gradient: 75%-95% B in 25 min; Flow rate: 60 mL/min; Detector: 254 nm; desired fractions were collected at 80% B) to afford the title compound (684 mg, 56% yield) as a yellow solid. LC/MS (ESI, m/z): [(M+H)]⁺=615.3.

### Step 6—N-[(1R,2S)-2-fluorocyclopropyl]-6-({2-methoxy-3-[4-(methoxymethylidene)cyclohexyl]phenyl}amino)-8-(methylamino)imidazo[1,2-b]pyridazine-3-carboxamide

To a stirred mixture of N-[(1R,2S)-2-fluorocyclopropyl]-6-({2-methoxy-3-[4-(methoxymethylidene)cyclohexyl]phenyl}amino)-8-{[(4-methoxyphenyl)methyl](methyl)amino}imidazo[1,2-b]pyridazine-3-carboxamide (684 mg, 1.11 mmol) in DCM (3 mL) was added TFA (2 mL) at rt. The resulting mixture was stirred for 30 min at rt. Next, the mixture was concentrated under reduced pressure. The residue was dissolved in TFA (4 mL) and H₂O (4 mL). The resulting mixture was stirred for 16 h at rt. On completion, the mixture was concentrated under reduced pressure. The residue was basified to pH 8 with NaOH. The resulting mixture was extracted with Et₂O (3×20 mL). The combined organic layers were washed with brine (3×10 mL), and dried over anhydrous Na₂SO₄. After filtration, the filtrate was concentrated under reduced pressure. The residue was purified by reverse phase Flash chromatography (Column: WelFlash™ C18-I, 20-40 um, 120 g; Eluent A: Water (plus 10 mmol/L FA); Eluent B: ACN; Gradient: 60%-70% B in 25 min; Flow rate: 60 mL/min; Detector: 254 nm; desired fractions were collected at 64% B) to afford the title compound (437 mg, 79% yield) as a white solid. LC/MS (ESI, m/z): [(M+H)]⁺=481.2.

### Example 1.302. Preparation of 6-{[3-(5-formylpyridin-2-yl)-2-methoxyphenyl]amino}-8-(methylamino)-N-(2-methylcyclopropyl)imidazo[1,2-b]pyridazine-3-carboxamide (Intermediate LF)

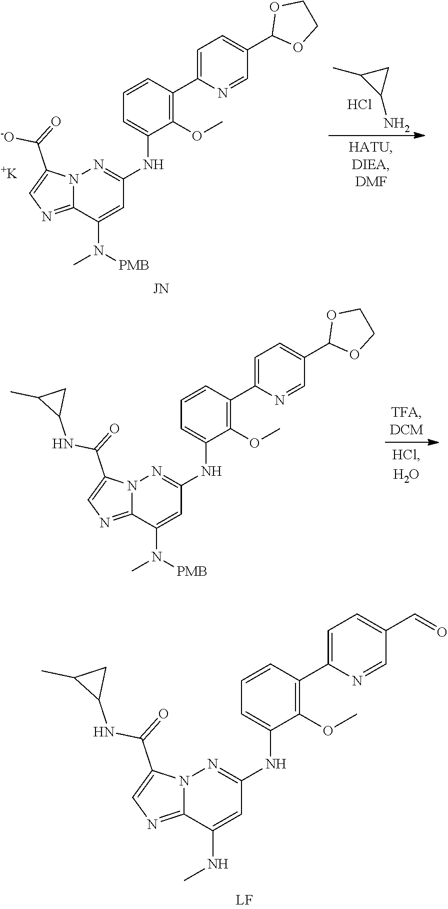

### Step 1—6-({3-[5-(1,3-Dioxolan-2-yl)pyridin-2-yl]-2-methoxyphenyl}amino)-8-{[(4-methoxyphenyl)methyl](methyl)amino}-N-(2-methylcyclopropyl)imidazo[1,2-b]pyridazine-3-carboxamide

To a stirred solution of 2-methylcyclopropan-1-amine hydrochloride (0.28 g, 2.6 mmol, CAS #89123-14-8) and 6-({3-[5-(1,3-dioxolan-2-yl)pyridin-2-yl]-2-methoxyphenyl}amino)-8-{[(4-methoxyphenyl)methyl](methyl)amino}imidazo[1,2-b]pyridazine-3-carboxylic acid potassium (1 g, 2 mmol, Intermediate JN) in DMF (10 mL) were added HATU (0.98 g, 2.6 mmol) and DIEA (0.60 mL, 3.4 mmol) in portions at rt under nitrogen atmosphere. The resulting mixture was stirred for 2 h at rt under nitrogen atmosphere. On completion, the mixture was purified by reverse phase Flash chromatography (Column: WelFlash™ C18-I, 20-40 um, 120 g; Eluent A: Water (plus 10 mmol/L NH₄HCO₃); Eluent B: ACN; Gradient: 40%-60% B in 25 min; Flow rate: 80 mL/min; Detector: 220/254 nm; desired fractions were collected at 50% B) to afford the title compound (420 mg, 39% yield) as a brown solid. LC/MS (ESI, m/z): [(M+H)]⁺=636.3.

### Step 2—6-{[3-(5-Formylpyridin-2-yl)-2-methoxyphenyl]amino}-8-(methylamino)-N-(2-methylcyclopropyl)imidazo[1,2-b]pyridazine-3-carboxamide

To a stirred solution of 6-({3-[5-(1,3-dioxolan-2-yl)pyridin-2-yl]-2-methoxyphenyl}amino)-8-{[(4-methoxyphenyl)methyl](methyl)amino}-N-(2-methylcyclopropyl)imidazo[1,2-b]pyridazine-3-carboxamide (400 mg, 0.6 mmol) in DCM (6 mL) were added TFA (2 mL) and 1 M aq. HCl (2 mL, 70 mmol) in portions at rt under nitrogen atmosphere. The resulting mixture was then stirred for 16 h at 50° C. under nitrogen atmosphere. On completion, the mixture was concentrated under reduced pressure. The residue was purified by reverse phase Flash chromatography (Column: WelFlash™ C18-I, 20-40 um, 120 g; Eluent A: Water (plus 10 mmol/L NH₄HCO₃); Eluent B: ACN; Gradient: 45%-75% B in 25 min; Flow rate: 80 mL/min; Detector: 220/254 nm; desired fractions were collected at 60% B) to afford the title compound (27.9 mg, 9% yield) as a brown solid. LC/MS (ESI, m/z): [(M+H)]⁺=472.3.

### Example 1.303. N-ethyl-6-{[3-(5-formylpyridin-2-yl)-2-methoxyphenyl]amino}-8-(methylamino)imidazo[1,2-b]pyridazine-3-carboxamide (Intermediate LG)

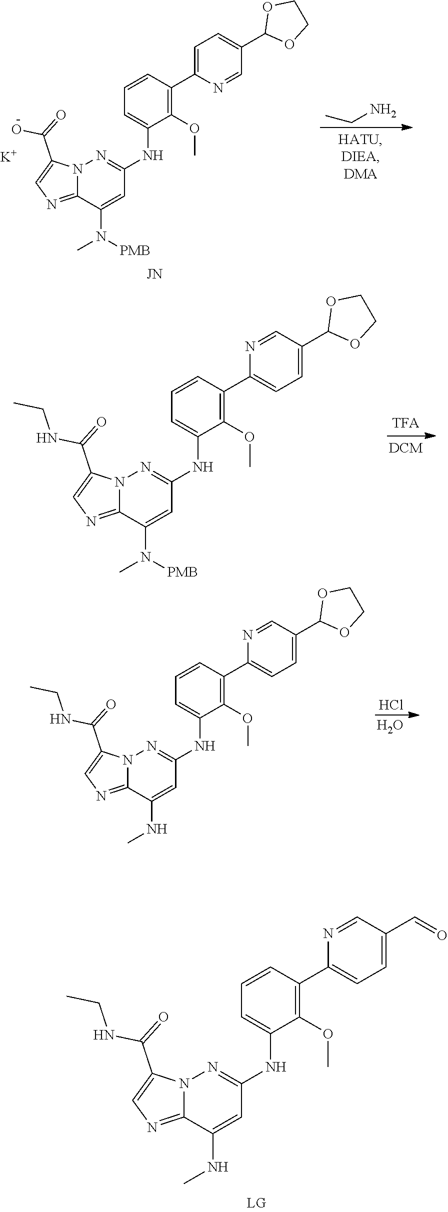

### Step 1—6-({3-[5-(1,3-Dioxolan-2-yl)pyridin-2-yl]-2-methoxyphenyl}amino)-N-ethyl-8-{[(4-methoxyphenyl)methyl](methyl)amino}imidazo[1,2-b]pyridazine-3-carboxamide

To a stirred solution of 6-({3-[5-(1,3-dioxolan-2-yl)pyridin-2-yl]-2-methoxyphenyl}amino)-8-{[(4-methoxyphenyl)methyl](methyl)amino}imidazo[1,2-b]pyridazine-3-carboxylic acid potassium (1 g, 2 mmol, Intermediate JN) and ethylamine (0.15 g, 3.2 mmol) in DMF (10 mL) were added DIEA (0.42 g, 3.2 mmol) and HATU (0.92 g, 2.4 mmol) at 0° C. under nitrogen atmosphere. The resulting mixture was then stirred for 1 h at rt under nitrogen atmosphere. On completion, the mixture was concentrated under reduced pressure. The residue was purified by reverse flash chromatography (Column: WelFlash™ C18-I, 20-40 μm, 330 g; Eluent A: Water (plus 10 mmol/L FA); Eluent B: ACN; Gradient: 40%-60% B in 30 min; Flow rate: 80 mL/min; Detector: 220/254 nm; desired fractions were collected at 55% B) to afford the title compound (767 mg, 78% yield) as an off-white solid. LC/MS (ESI, m/z): [(M+H)]⁺=610.2.

### Step 2—6-({3-[5-(1,3-Dioxolan-2-yl)pyridin-2-yl]-2-methoxyphenyl}amino)-N-ethyl-8-(methylamino)imidazo[1,2-b]pyridazine-3-carboxamide

To a stirred solution of 6-({3-[5-(1,3-dioxolan-2-yl)pyridin-2-yl]-2-methoxyphenyl}amino)-N-ethyl-8-{[(4-methoxyphenyl)methyl](methyl)amino}imidazo[1,2-b]pyridazine-3-carboxamide (760 mg, 1.247 mmol) in DCM (10 mL) was added a solution of TFA (5 mL) at 0° C. under nitrogen atmosphere. The resulting mixture was stirred for 2 h at rt under nitrogen atmosphere. On completion, the mixture was concentrated under reduced pressure. The residue was triturated with Et₂O to afford the title compound (700 mg) as a brown solid. LC/MS (ESI, m/z): [(M+H)]⁺=490.3.

### Step 3—N-ethyl-6-{[3-(5-formylpyridin-2-yl)-2-methoxyphenyl]amino}-8-(methylamino)imidazo[1,2-b]pyridazine-3-carboxamide

To a stirred solution of 6-({3-[5-(1,3-dioxolan-2-yl)pyridin-2-yl]-2-methoxyphenyl}amino)-N-ethyl-8-(methylamino)imidazo[1,2-b]pyridazine-3-carboxamide (700 mg, 1 mmol) in H₂O (10 mL) was added a solution of HCl (6 mL, 200 mmol) at 0° C. under nitrogen atmosphere. The resulting mixture was stirred for 2 h at 50° C. under nitrogen atmosphere. On completion, the mixture was cooled to rt and concentrated under reduced pressure. The residue was purified by reverse phase flash chromatography (Column: WelFlash™ C18-I, 20-40 μm, 330 g; Eluent A: Water (plus 10 mmol/L FA); Eluent B: ACN; Gradient: 50%-70% B in 20 min; Flow rate: 80 mL/min; Detector: 220/254 nm; desired fractions were collected at 65% B) to afford the title compound (21.8 mg, 3% yield) as an off-white solid. ¹H NMR (400 MHz, DMSO-d₆) δ 10.17 (s, 1H), 9.24-9.19 (m, 1H), 8.76 (t, J=5.8 Hz, 1H), 8.66 (s, 1H), 8.34 (dd, J=8.1, 2.2 Hz, 1H), 8.08 (d, J=8.2 Hz, 1H), 7.91-7.84 (m, 1H), 7.83-7.77 (m, 1H), 7.59-7.52 (m, 1H), 7.52-7.44 (m, 1H), 7.31 (t, J=7.9 Hz, 1H), 6.12 (s, 1H), 3.56 (s, 3H), 3.28-3.19 (m, 2H), 2.91 (d, J=4.8 Hz, 3H), 0.98 (t, J=7.2 Hz, 3H). LC/MS (ESI, m/z): [(M+H)]⁺=446.1.

### Example 1.304. Preparation of 3-(5-(4-(3,9-diazaspiro[5.5]undecan-3-yl)phenyl)-3-methyl-2-oxo-2,3-dihydro-1H-benzo[d]imidazol-1-yl)piperidine-2,6-dione (Intermediate LH)

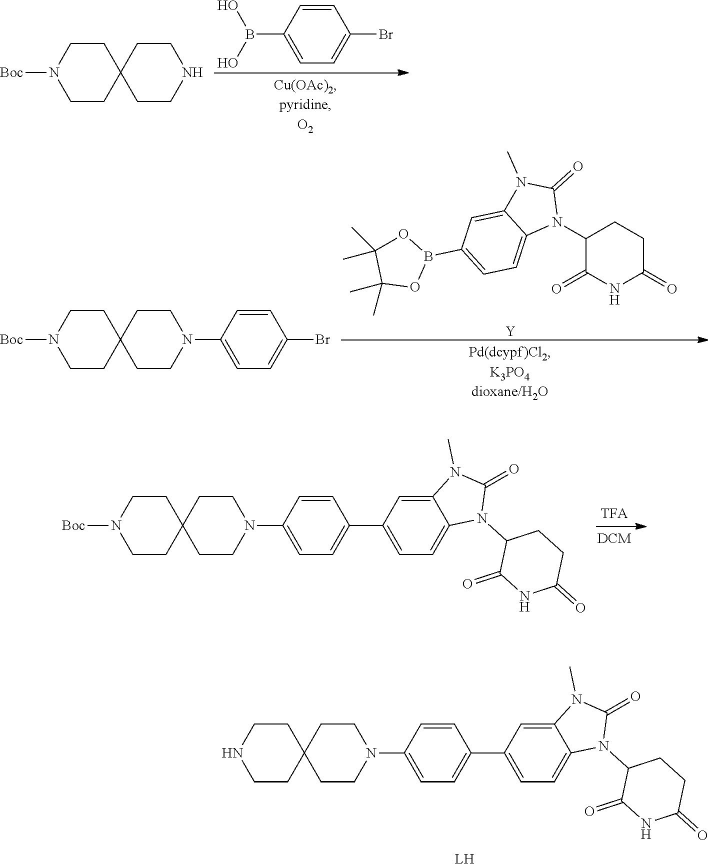

### Step 1—Tert-butyl 9-(4-bromophenyl)-3,9-diazaspiro[5.5]undecane-3-carboxylate

To a stirred solution of tert-butyl 3,9-diazaspiro[5.5]undecane-3-carboxylate (3 g, 10 mmol) and 4-bromophenylboric acid (2.84 g, 14.2 mmol) in pyridine (30 mL) was added Cu(OAc)₂ (3.21 g, 17.7 mmol) in portions at rt under nitrogen atmosphere. The resulting mixture was then stirred for 16 h at 40° C. under oxygen atmosphere. On completion, the mixture was cooled to rt and the precipitated solids were collected by filtration and washed with EtOAc (2×20 mL). The filtrate was then concentrated under reduced pressure. The crude product was purified by silica gel column chromatography with the following conditions (Mobile Phase A: PE, Mobile Phase B: EA; Gradient: 0% B to 50% B in 25 min, 254 nm; the fractions containing the desired product were collected at 30% B) to afford the title compound (3.73 g, 77% yield) as a brown oil. LC/MS (ESI, m/z): [(M+H)]⁺=409.2, 411.2.

### Step 2—Tert-butyl 9-{4-[1-(2,6-dioxopiperidin-3-yl)-3-methyl-2-oxo-1,3-benzodiazol-5-yl]phenyl}-3,9-diazaspiro[5.5]undecane-3-carboxylate

To a stirred solution of tert-butyl 9-(4-bromophenyl)-3,9-diazaspiro[5.5]undecane-3-carboxylate (1 g, 2 mmol) and 3-[3-methyl-2-oxo-5-(4,4,5,5-tetramethyl-1,3,2-dioxaborolan-2-yl)-1,3-benzodiazol-1-yl]piperidine-2,6-dione (0.94 g, 2.4 mmol, Intermediate Y) in dioxane (10 mL) were added H₂O (1 mL) and K₃PO₄ (1.56 g, 7.33 mmol) in portions at rt under nitrogen atmosphere. To the above mixture was added [1,1′-Bis(di-cyclohexylphosphino)ferrocene]dichloropalladium(II) (181.44 mg, 0.24 mmol, CAS #917511-90-1) in portions rt. The resulting mixture was then stirred for 30 min at 80° C. under nitrogen atmosphere. On completion, the mixture was cooled to rt and the residue was purified by reverse phase flash chromatography (Column: WelFlash™ C18-I, 20-40 um, 120 g; Eluent A: Water (plus 10 mmol/L NH₄HCO₃); Eluent B: ACN; Gradient: 35%-90% B in 35 min; Flow rate: 80 mL/min; Detector: 220/254 nm; desired fractions were collected at 87% B) to afford the title compound (740 mg, 52% yield) as a yellow solid. LC/MS (ESI, m/z): [(M+H)]⁺=588.4.

### Step 3—3-(5-(4-(3,9-diazaspiro[5.5]undecan-3-yl)phenyl)-3-methyl-2-oxo-2,3-dihydro-1H-benzo[d]imidazol-1-yl)piperidine-2,6-dione

To a stirred solution of tert-butyl 9-{4-[1-(2,6-dioxopiperidin-3-yl)-3-methyl-2-oxo-1,3-benzodiazol-5-yl]phenyl}-3,9-diazaspiro[5.5]undecane-3-carboxylate (700 mg, 1 mmol) in DCM (20 mL) was added TFA (4 mL, 50 mmol) dropwise at rt under nitrogen atmosphere. The resulting mixture was stirred for 2 h at rt under nitrogen atmosphere. On completion, the mixture was purified by reverse phase flash chromatography (Column: WelFlash™ C18-I, 20-40 um, 120 g; Eluent A: Water (plus 10 mmol/L NH₄HCO₃); Eluent B: ACN; Gradient: 25%-55% B in 25 min; Flow rate: 80 mL/min; Detector: 220/254 nm; desired fractions were collected at 50% B) to afford the title compound (500 mg, 86% yield) as a white solid LC/MS (ESI, m/z): [(M+H)]⁺=488.2.

### Example 1.305. Preparation of 6-[(2-Chloro-3-formylphenyl)amino]-N-[(1R,2S)-2-fluorocyclopropyl]-8-(methylamino)imidazo[1,2-b]pyridazine-3-carboxamide (Intermediate LI)

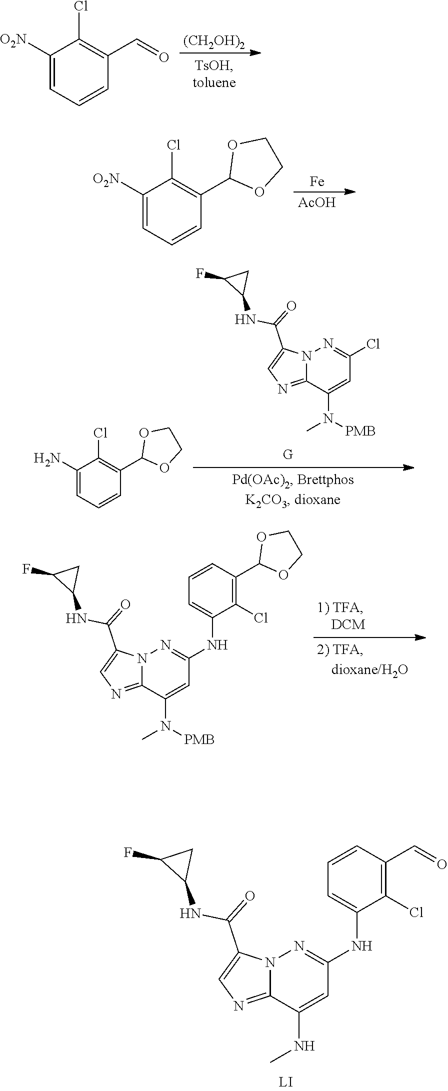

### Step 1—2-(2-Chloro-3-nitrophenyl)-1,3-dioxolane

To a stirred solution of 2-chloro-3-nitrobenzaldehyde (5 g, 30 mmol, CAS #: 58755-57-0) and ethylene glycol (6.69 g, 108 mmol) in toluene (100 mL) was added TsOH (0.46 g, 2.7 mmol) at rt under nitrogen atmosphere. The resulting mixture was stirred for 16 h at 110° C. under nitrogen atmosphere. On completion, the mixture was cooled to rt and was basified to pH 10 with saturated NaHCO₃ (aq.). The aqueous layer was extracted with EtOAc (100 mL). The resulting mixture was concentrated under reduced pressure. The residue was purified by silica gel column chromatography, eluted with PE/EA (3:1), to afford the title compound (5.0 g, 81% yield) as a brown oil. LC/MS (ESI, m/z): [(M+H)]⁺=230.2.

### Step 2—2-Chloro-3-(1,3-dioxolan-2-yl)aniline

To a stirred solution of 2-(2-chloro-3-nitrophenyl)-1,3-dioxolane (2.0 g, 8.7 mmol) and NH₄Cl (0.93 g, 17 mmol) in THF (20 mL) and H₂O (10 mL) was added Fe (2.43 g, 43.6 mmol) at rt under nitrogen atmosphere. The resulting mixture was stirred for 2 h at 70° C. under nitrogen atmosphere. On completion, the mixture was cooled to rt and mixture was filtered, then the filter cake was washed with DMF (10 mL). The filtrate was concentrated under reduced pressure to afford the title compound (1.7 g, 98% yield) as a brown solid. LC/MS (ESI, m/z): [(M+H)]⁺=200.1.

### Step 3—6-{[2-Chloro-3-(1,3-dioxolan-2-yl)phenyl]amino}-N-[(1R,2S)-2-fluorocyclopropyl]-8-{[(4-methoxyphenyl)methyl](methyl)amino}imidazo[1,2-b]pyridazine-3-carboxamide

To a stirred solution of 2-chloro-3-(1,3-dioxolan-2-yl)aniline (1.0 g, 5.009 mmol) and 6-chloro-N-[(1R,2S)-2-fluorocyclopropyl]-8-{[(4-methoxyphenyl)methyl](methyl)amino}imidazo[1,2-b]pyridazine-3-carboxamide (2.43 g, 6.01 mmol, Intermediate G) in 1,4-dioxane (10 mL) were added K₂CO₃ (1.38 g, 10.0 mmol), Pd(OAc)₂ (0.11 g, 0.50 mmol) and BrettPhos (0.27 g, 0.50 mmol) in portions at rt under nitrogen atmosphere. The resulting mixture was stirred for 16 h at 80° C. under nitrogen atmosphere. On completion, the mixture was cooled to rt and the residue was purified by reverse phase flash chromatography (Column: WelFlash™ C18-I, 20-40 um, 330 g; Eluent A: Water (plus 10 mmol/L NH₄HCO₃); Eluent B: ACN; Gradient: 25%-55% B in 25 min; Flow rate: 80 mL/min; Detector: 220/254 nm; desired fractions were collected at 50% B) to afford the title compound (500 mg, 18% yield) as an off-white solid. LC/MS (ESI, m/z): [(M+H)]⁺=567.2.

### Step 4—6-[(2-Chloro-3-formylphenyl)amino]-N-[(1R,2S)-2-fluorocyclopropyl]-8-(methylamino)imidazo[1,2-b]pyridazine-3-carboxamide

To a stirred solution of 6-{[2-chloro-3-(1,3-dioxolan-2-yl)phenyl]amino}-N-[(1R,2S)-2-fluorocyclopropyl]-8-{[(4-methoxyphenyl)methyl](methyl)amino}imidazo[1,2-b]pyridazine-3-carboxamide (500 mg, 0.9 mmol) in DCM (2 mL) were added TFA (0.20 mL, 2.7 mmol) and HCl (0.08 mL, 3 mmol) in portions at rt under nitrogen atmosphere. The resulting mixture was stirred for 16 h at 50° C. under nitrogen atmosphere. On completion, the mixture was concentrated under reduced pressure. The residue was purified by reverse phase flash chromatography (Column: WelFlash™ C18-I, 20-40 um, 120 g; Eluent A: Water (plus 10 mmol/L NH₄HCO₃); Eluent B: ACN; Gradient: 45%-65% B in 25 min; Flow rate: 80 mL/min; Detector: 220/254 nm; desired fractions were collected at 50% B) to afford the title compound (200 mg, 56% yield) as a white solid. LC/MS (ESI, m/z): [(M+H)]⁺=403.1.

### Example 1.306. 6-{[2-(difluoromethyl)-3-(5-formylpyridin-2-yl)phenyl]amino}-N-[(1R,2S)-2-fluorocyclopropyl]-8-(methylamino)imidazo[1,2-b]pyridazine-3-carboxamide (Intermediate LJ)

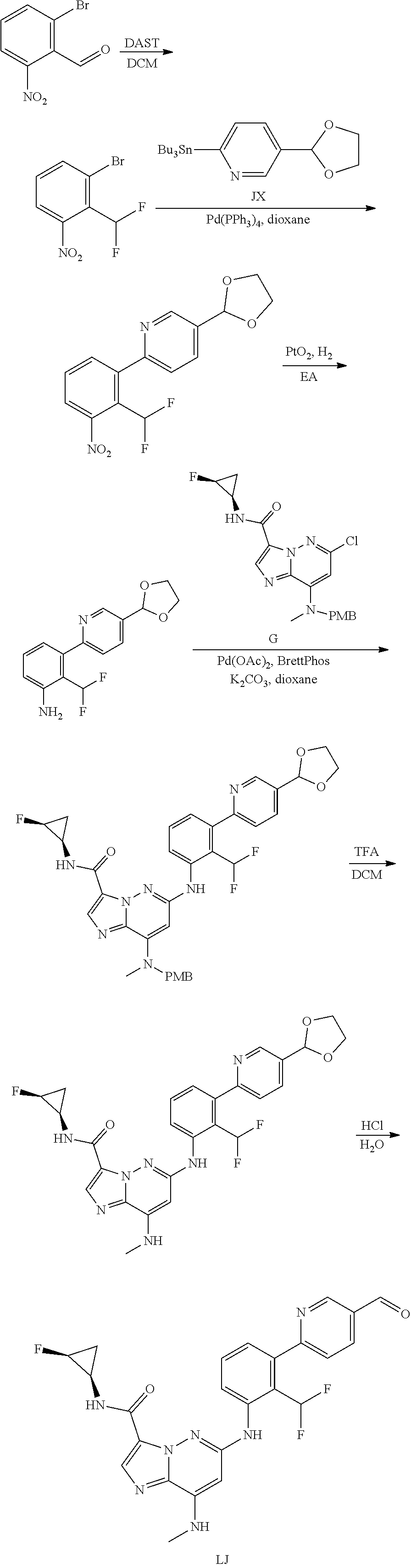

### Step 1—1-Bromo-2-(difluoromethyl)-3-nitrobenzene

To a stirred solution of 2-bromo-6-nitrobenzaldehyde (4 g, 20 mmol, CAS #: 20357-21-5) in DCM (20 mL) was added DAST (8.41 g, 52.2 mmol) dropwise at 0° C. under nitrogen atmosphere. The resulting mixture was then stirred for 16 h at rt under nitrogen atmosphere. On completion, the reaction was quenched by the addition of ice water (100 mL) at 0° C. The resulting mixture was extracted with EtOAc (300 mL). The combined organic layers were washed with brine (300 mL), and dried over anhydrous Na₂SO₄. After filtration, the filtrate was concentrated under reduced pressure. The residue was purified by silica gel column chromatography, eluted with PE/EA (5:1), to afford the title compound (2 g, 46% yield) as an off-white solid. LC/MS (ESI, m/z): [(M+H)]⁺=251.9, 253.9.

### Step 2—2-[2-(Difluoromethyl)-3-nitrophenyl]-5-(1,3-dioxolan-2-yl)pyridine

To a stirred solution of 1-bromo-2-(difluoromethyl)-3-nitrobenzene (2 g, 8 mmol) in dioxane (20 mL) was added 5-(1,3-dioxolan-2-yl)-2-(tributylstannyl)pyridine (4.19 g, 9.52 mmol, Intermediate JX) at rt under nitrogen atmosphere. To the above mixture was added Pd(PPh₃)₄ (0.92 g, 0.79 mmol) at rt under nitrogen atmosphere. The resulting mixture was stirred for additional 5 min at rt. Then the mixture was stirred for 16 h at 100° C. under nitrogen atmosphere. On completion, the mixture was cooled to rt and concentrated under reduced pressure. The residue was purified by silica gel column chromatography, eluted with PE/EA (3:1), to afford the title compound (1 g, 39% yield) as a light brown oil. LC/MS (ESI, m/z): [(M+H)]⁺=323.0.

### Step 3—2-(Difluoromethyl)-3-[5-(1,3-dioxolan-2-yl)pyridin-2-yl]aniline

To a solution of 2-[2-(difluoromethyl)-3-nitrophenyl]-5-(1,3-dioxolan-2-yl)pyridine (1 g, 3 mmol) in THF (20 mL) was added PtO₂ (70.46 mg, 0.310 mmol) under nitrogen atmosphere. The reaction system was degassed and purged with H₂ several times, then it was hydrogenated under H₂ balloon (˜1 atm) at 25° C. for 2 h. After completion of the reaction, Pd/C was filtered off through celite. The filter cake was washed with THF (3×10 mL). The corresponding filtrate was concentrated under reduced pressure to provide the title compound (700 mg, 77% yield) as a colorless oil. LC/MS (ESI, m/z): [(M+H)]⁺=293.1.

### Step 4—6-{[2-(Difluoromethyl)-3-[5-(1,3-dioxolan-2-yl)pyridin-2-yl]phenyl]amino}-N-[(1R,2S)-2-fluorocyclopropyl]-8-{[(4-methoxyphenyl)methyl](methyl)amino}imidazo[1,2-b]pyridazine-3-carboxamide

To a stirred solution of 2-(difluoromethyl)-3-[5-(1,3-dioxolan-2-yl)pyridin-2-yl]aniline (650 mg, 2.224 mmol) and 6-chloro-N-[(1R,2S)-2-fluorocyclopropyl]-8-{[(4-methoxyphenyl)methyl](methyl)amino}imidazo[1,2-b]pyridazine-3-carboxamide (898.08 mg, 2.224 mmol, Intermediate G) in dioxane (5 mL) were added BrettPhos (119.37 mg, 0.222 mmol) and K₂CO₃ (922.04 mg, 6.672 mmol) at rt under argon atmosphere. The resulting mixture was stirred for additional 5 min at rt. To the above mixture was added Pd(AcO)₂ (49.93 mg, 0.222 mmol) in portions over 5 min at rt. The resulting mixture was stirred for additional 30 min at rt. Then the mixture was stirred for 16 h at 90° C. under nitrogen atmosphere. On completion, the mixture was cooled to rt and concentrated under reduced pressure. The residue was purified by reverse phase flash chromatography (Column: WelFlash™ C18-I, 20-40 μm, 120 g; Eluent A: Water (plus 5 mmol/L NH₄HCO₃); Eluent B: ACN; Gradient: 40%-75% B in 35 min; Flow rate: 60 mL/min; Detector: 220/254 nm; desired fractions were collected at 70% B) to afford the title compound (800 mg, 55% yield) as a light brown solid. LC/MS (ESI, m/z): [(M+H)]⁺=660.2.

### Step 5—6-{[2-(Difluoromethyl)-3-[5-(1,3-dioxolan-2-yl)pyridin-2-yl]phenyl]amino}-N-[(1R,2S)-2-fluorocyclopropyl]-8-(methylamino)imidazo[1,2-b]pyridazine-3-carboxamide

To a solution of 6-{[2-(difluoromethyl)-3-[5-(1,3-dioxolan-2-yl)pyridin-2-yl]phenyl]amino}-N-[(1R,2S)-2-fluorocyclopropyl]-8-{[(4-methoxyphenyl)methyl](methyl)amino}imidazo[1,2-b]pyridazine-3-carboxamide (200 mg, 0.3 mmol) in THF (5 mL) was added TFA (2 mL) dropwise at 0° C. under nitrogen atmosphere. The reaction mixture was stirred for 2 h at rt under nitrogen atmosphere. On completion, the mixture was concentrated under reduced pressure. The residue was triturated with Et₂O to afford the title compound (150 mg, 92% yield) as an off-white solid. LC/MS (ESI, m/z): [(M+H)]⁺=540.2.

### Step 6—6-{[2-(Difluoromethyl)-3-(5-formylpyridin-2-yl)phenyl]amino}-N-[(1R,2S)-2-fluorocyclopropyl]-8-(methylamino)imidazo[1,2-b]pyridazine-3-carboxamide

A solution of 6-{[2-(difluoromethyl)-3-[5-(1,3-dioxolan-2-yl)pyridin-2-yl]phenyl]amino}-N-[(1R,2S)-2-fluorocyclopropyl]-8-(methylamino)imidazo[1,2-b]pyridazine-3-carboxamide (50 mg, 0.09 mmol) in 2 M aq. HCl (2 mL) was stirred for 16 h at 50° C. under nitrogen atmosphere. On completion, the mixture was cooled to rt and basified to pH 8 with NaOH. The resulting mixture was concentrated under reduced pressure. The residue was purified by reverse phase flash chromatography (Column: WelFlash™ C18-I, 20-40 μm, 120 g; Eluent A: Water (plus 5 mmol/L NH₄HCO₃); Eluent B: ACN; Gradient: 30%-65% B in 35 min; Flow rate: 60 mL/min; Detector: 220/254 nm; desired fractions were collected at 50% B) to afford the title compound (12.7 mg, 28% yield) as an off-white solid. LC/MS (ESI, m/z): [(M+H)]⁺=496.2.

### Example 1.307. Preparation of 1-(3-{3,9-Diazaspiro[5.5]undecane-3-carbonyl}phenyl)-1,3-diazinane-2,4-dione (Intermediate LK)

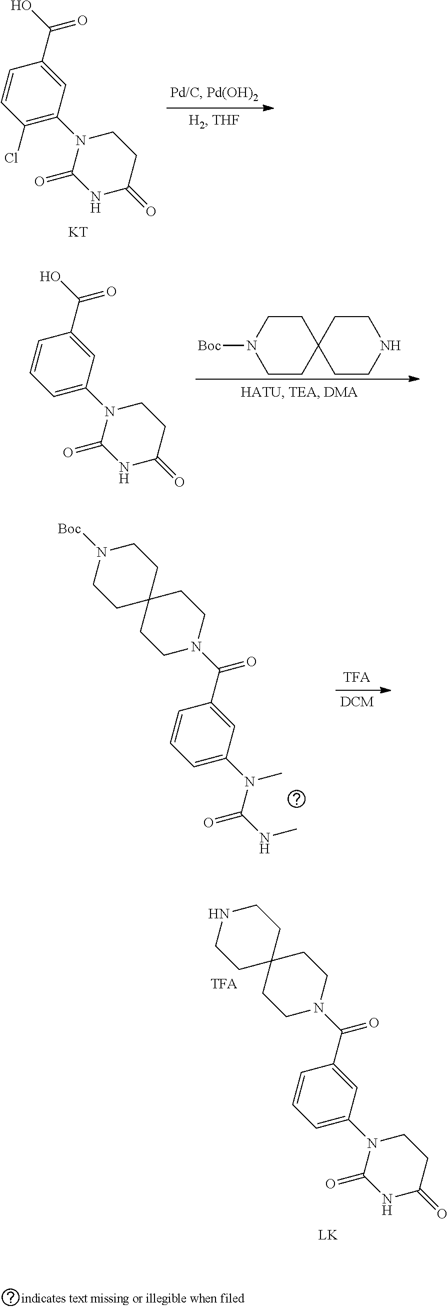

### Step 1—3-(2,4-Dioxo-1,3-diazinan-1-yl)benzoic Acid

To a solution of 4-chloro-3-(2,4-dioxo-1,3-diazinan-1-yl)benzoic acid (500 mg, 1.861 mmol, Intermediate KT) in THF (10 mL) was added Pd/C (10 wt %, 250 mg) in a pressure tank. The mixture was hydrogenated at rt under 30 psi of hydrogen pressure for 16 h. On completion, the mixture was filtered, and the filter cake was washed with DMF (10 mL). The filtrate was concentrated under reduced pressure to give the title compound (430 mg, 99% yield) as a brown solid. LC/MS (ESI, m/z): [(M+H)]⁺=235.1.

### Step 2—Tert-butyl 9-[3-(2,4-dioxo-1,3-diazinan-1-yl)benzoyl]-3,9-diazaspiro[5.5]undecane-3-carboxylate

To a stirred solution of 3-(2,4-dioxo-1,3-diazinan-1-yl)benzoic acid (300 mg, 1 mmol) and tert-butyl 3,9-diazaspiro[5.5]undecane-3-carboxylate (325.83 mg, 1.281 mmol) in DMA (4 mL) were added HATU (730.56 mg, 1.921 mmol) and TEA (0.53 mL, 3.843 mmol) in portions at room temperature under nitrogen atmosphere. The resulting mixture was stirred for 16 h at rt under nitrogen atmosphere. On completion, the mixture was purified by reverse phase flash chromatography (Column: WelFlash™ C18-I, 20-40 um, 120 g; Eluent A: Water (plus 10 mmol/L TFA); Eluent B: ACN; Gradient: 25%-55% B in 25 min; Flow rate: 80 mL/min; Detector: 220/254 nm; desired fractions were collected at 34% B) to afford the title compound (490 mg, 81% yield) as a brown oil. LC/MS (ESI, m/z): [(M+H)]⁺=471.3.

### Step 3—1-(3-{3,9-Diazaspiro[5.5]undecane-3-carbonyl}phenyl)-1,3-diazinane-2,4-dione Trifluoroacetate

To a stirred solution of 3-(2,4-dioxo-1,3-diazinan-1-yl)benzoic acid (300 mg, 1 mmol) and tert-butyl 3,9-diazaspiro[5.5]undecane-3-carboxylate (325.83 mg, 1.281 mmol) in DCM (5 mL) were added TFA (2 mL) dropwise at rt. The resulting mixture was stirred for 1 h at rt. On completion, the mixture was concentrated under vacuum. The residue was triturated with Et₂O to afford the title compound (350 mg, 72% yield) as a brown solid. LC/MS (ESI, m/z): [(M+H)]⁺=371.1.

### Example 1.308. Preparation of N-[(1R,2S)-2-fluorocyclopropyl]-8-(methylamino)-6-{4-[5-(piperazine-1-carbonyl)pyridin-2-yl]-2,3-dihydroindol-1-yl}imidazo[1,2-b]pyridazine-3-carboxamide (Intermediate LL)

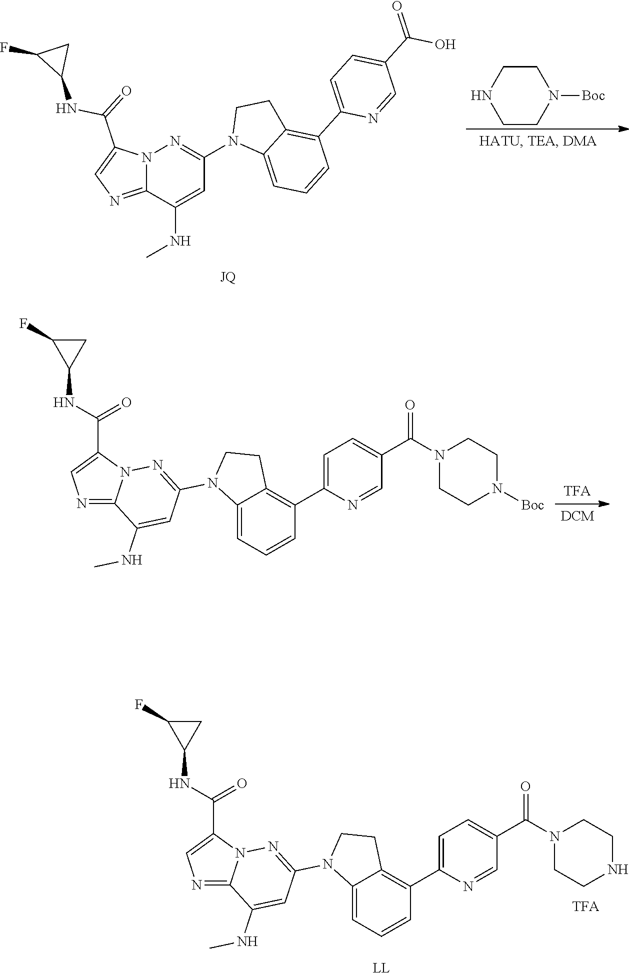 [SMILES: CNc1cc(N2CCc3c(-c4ccc(C(=O)O)cn4)cccc32)nn2c(C(=O)N[C@@H]3C[C@@H]3F)cnc12 ; CC(C)(C)OC(=O)N1CCNCC1 ; CNc1cc(N2CCc3c(-c4ccc(C(=O)N5CCN(C(=O)OC(C)(C)C)CC5)cn4)cccc32)nn2c(C(=O)N[C@@H]3C[C@@H]3F)cnc12 ; CNc1cc(N2CCc3c(-c4ccc(C(=O)N5CCNCC5)cn4)cccc32)nn2c(C(=O)N[C@@H]3C[C@@H]3F)cnc12]

### Step 1—Tert-butyl 4-{6-[1-(3-{[(1R,2S)-2-fluorocyclopropyl]carbamoyl}-8-(methylamino)imidazo[1,2-b]pyridazin-6-yl)-2,3-dihydroindol-4-yl]pyridine-3-carbonyl}piperazine-1-carboxylate

To a stirred mixture of 6-[1-(3-{[(1R,2S)-2-fluorocyclopropyl]carbamoyl}-8-(methylamino)imidazo[1,2-b]pyridazin-6-yl)-2,3-dihydroindol-4-yl]pyridine-3-carboxylic acid (300 mg, 0.6 mmol, Intermediate JQ) and tert-butyl piperazine-1-carboxylate (138 mg, 0.741 mmol) in DMA (3 mL) were added HATU (468 mg, 1.23 mmol) and TEA (186 mg, 1.84 mmol) at rt under nitrogen atmosphere. The resulting mixture was stirred for 1 h at rt under nitrogen atmosphere. On completion, the mixture was purified by reversed-phase flash chromatography (column, C18 silica gel; mobile phase B:ACN; mobile phase A: Water (10 mmol/L NH₄HCO₃), 40% to 60% gradient in 25 min; detector, UV 254 nm; the fractions were collected at 48%) to afford the title compound (320 mg, 79% yield) as a white solid. LC/MS (ESI, m/z): [(M+H)]⁺=656.4.

### Step 2—N-[(1R,2S)-2-fluorocyclopropyl]-8-(methylamino)-6-{4-[5-(piperazine-1-carbonyl)pyridin-2-yl]-2,3-dihydroindol-1-yl}imidazo[1,2-b]pyridazine-3-carboxamide Trifluoroacetate

A solution of tert-butyl 4-{6-[1-(3-{[(1R,2S)-2-fluorocyclopropyl]carbamoyl}-8-(methylamino)imidazo[1,2-b]pyridazin-6-yl)-2,3-dihydroindol-4-yl]pyridine-3-carbonyl}piperazine-1-carboxylate (320 mg, 0.43 mmol) in DCM (2 mL) was added TFA (2 mL) dropwise at rt. The reaction mixture was stirred for 1 h at rt under nitrogen atmosphere. On completion, the mixture was concentrated under vacuum. The residue was triturated with Et₂O to afford the title compound (350 mg) as a white solid. LC/MS (ESI, m/z): [(M+H)]⁺=556.3.

### Example 1.309. Preparation of 6-(3-{[3-(1-Isopropyl-1,2,3-triazol-4-yl)-8-(methylamino)imidazo[1,2-b]pyridazin-6-yl]amino}-2-methoxyphenyl)pyridine-3-carbaldehyde (Intermediate LM)

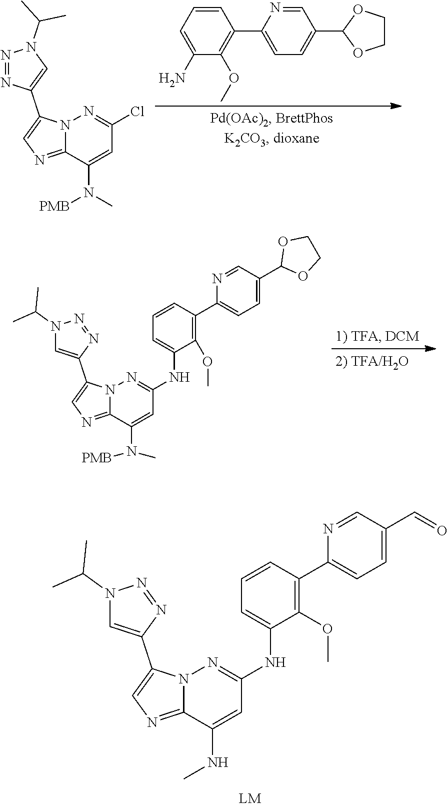

### Step 1—N6-{3-[5-(1,3-dioxolan-2-yl)pyridin-2-yl]-2-methoxyphenyl}-3-(1-isopropyl-1,2,3-triazol-4-yl)-N8-[(4-methoxyphenyl)methyl]-N8-methylimidazo[1,2-b]pyridazine-6,8-diamine

To a stirred solution of 6-chloro-3-(1-isopropyl-1,2,3-triazol-4-yl)-N-[(4-methoxyphenyl)methyl]-N-methylimidazo[1,2-b]pyridazin-8-amine (700 mg, 2 mmol, synthesized via Steps 1-3 of Intermediate KI) and 3-[5-(1,3-dioxolan-2-yl)pyridin-2-yl]-2-methoxyaniline (555.33 mg, 2.039 mmol, synthesized via Steps 1˜4 of Intermediate IS) in dioxane (10 mL) were added K₂CO₃ (704.63 mg, 5.097 mmol) and Pd(AcO)₂ (38.16 mg, 0.170 mmol) in portions at rt under nitrogen atmosphere. To the above mixture was added BrettPhos (91.23 mg, 0.170 mmol) in portions. The resulting mixture was stirred for additional 16 h at 110° C. On completion, the mixture was cooled to rt and concentrated under reduced pressure. The residue was purified by reverse phase flash chromatography (Column: WelFlash™ C18-I, 20-40 um, 120 g; Eluent A: Water (10 mmol/L NH₄HCO₃); Eluent B: ACN; Gradient: 30%-70% B in 30 min; Flow rate: 80 mL/min; Detector: 254 nm; desired fractions were collected at 69% B) to afford the title compound (900 mg, 82% yield) as a brown solid. LC/MS (ESI, m/z): [(M+H)]⁺=648.3.

### Step 2—6-(3-{[3-(1-Isopropyl-1,2,3-triazol-4-yl)-8-(methylamino)imidazo[1,2-b]pyridazin-6-yl]amino}-2-methoxyphenyl)pyridine-3-carbaldehyde

To a stirred solution of N6-{3-[5-(1,3-dioxolan-2-yl)pyridin-2-yl]-2-methoxyphenyl}-3-(1-isopropyl-1,2,3-triazol-4-yl)-N8-[(4-methoxyphenyl)methyl]-N8-methylimidazo[1,2-b]pyridazine-6,8-diamine (900 mg, 1 mmol) in DCM (10 mL) was added TFA (5 mL) dropwise at rt under nitrogen atmosphere. The resulting mixture was stirred for 2 h at rt under nitrogen atmosphere. Then the mixture was concentrated under reduced pressure. To the above mixture was added H₂O (10 mL) in portions at rt. The resulting mixture was stirred for 2 h at 50° C. under nitrogen atmosphere. On completion, the mixture was cooled to rt and purified by reverse phase flash chromatography (Column: WelFlash™ C18-I, 20-40 um, 120 g; Eluent A: Water (plus 10 mmol/L NH₄HCO₃); Eluent B: ACN; Gradient: 25%-55% B in 25 min; Flow rate: 80 mL/min; Detector: 220/254 nm; desired fractions were collected at 42% B) to afford the title compound (200 mg, 30% yield) as an off-white solid. LC/MS (ESI, m/z): [(M+H)]⁺=484.2.

### Example 1.310. Preparation of 6-{1-[3-(1-isopropyl-1,2,3-triazol-4-yl)-8-(methylamino)imidazo[1,2-b]pyridazin-6-yl]-2,3-dihydroindol-4-yl}pyridine-3-carbaldehyde (Intermediate LN)

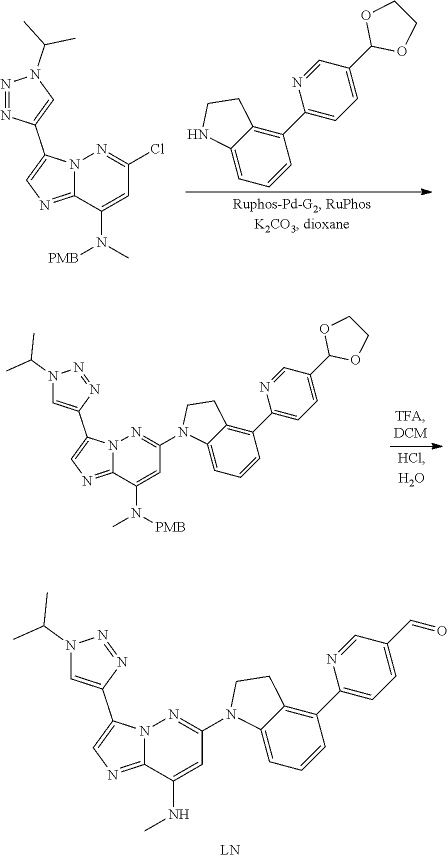

### Step 1—6-{4-[5-(1,3-Dioxolan-2-yl)pyridin-2-yl]-2,3-dihydroindol-1-yl}-3-(1-isopropyl-1,2,3-triazol-4-yl)-N-[(4-methoxyphenyl)methyl]-N-methylimidazo[1,2-b]pyridazin-8-amine

To a stirred solution of 6-chloro-3-(1-isopropyl-1,2,3-triazol-4-yl)-N-[(4-methoxyphenyl)methyl]-N-methylimidazo[1,2-b]pyridazin-8-amine (750 mg, 1.8 mmol, synthesized via Steps 1-3 of Intermediate KI) and 4-[5-(1,3-dioxolan-2-yl)pyridin-2-yl]-2,3-dihydro-1H-indole (488.57 mg, 1.821 mmol, synthesized via Steps 1-3 of Intermediate IN) in 1,4-dioxane (10 mL) were added K₂CO₃ (754.96 mg, 5.463 mmol) and RuPhos (84.97 mg, 0.182 mmol) in portions at rt under nitrogen atmosphere. To the above mixture was added RuPhos-PdCl-2nd G (141.62 mg, 0.182 mmol) at rt under nitrogen atmosphere. The resulting mixture was stirred for additional 16 h at 100° C. On completion, the mixture was cooled to rt and concentrated under reduced pressure. The residue was purified by reverse phase flash chromatography (Column: WelFlash™ C18-I, 20-40 um, 330 g; Eluent A: Water (plus 10 mmol/L NH₄HCO₃); Eluent B: ACN; Gradient: 35%-75% B in 25 min; Flow rate: 80 mL/min; Detector: 254 nm; desired fractions were collected at 72% B) to afford the title compound (1.06 g, 90% yield) as a brown solid. LC/MS (ESI, m/z): [(M+H)]⁺=644.3.

### Step 2—6-{1-[3-(1-Isopropyl-1,2,3-triazol-4-yl)-8-(methylamino)imidazo[1,2-b]pyridazin-6-yl]-2,3-dihydroindol-4-yl}pyridine-3-carbaldehyde

To a stirred solution of 6-{4-[5-(1,3-dioxolan-2-yl)pyridin-2-yl]-2,3-dihydroindol-1-yl}-3-(1-isopropyl-1,2,3-triazol-4-yl)-N-[(4-methoxyphenyl)methyl]-N-methylimidazo[1,2-b]pyridazin-8-amine (1.09 g, 1.693 mmol) in DCM (10 mL) was added TFA (5 mL) at rt under nitrogen atmosphere. The resulting mixture was stirred for 2 h at rt under nitrogen atmosphere. Then the mixture was concentrated under reduced pressure. The residue was dissolved in H₂O (10 mL). To the above mixture was added 2 M HCl (2 mL) at rt. The resulting mixture was stirred for additional 2 h at 60° C. On completion, the mixture was concentrated under reduced pressure. The mixture was basified to pH 8-9 with 2 M NaOH. The precipitated solids were collected by filtration and washed with H₂O (3×10 mL) to afford the title compound (700 mg, 86% yield) as a brown solid. LC/MS (ESI, m/z): [(M+H)]⁺=480.2.

### Example 1.311. Preparation of N-[(2S)-2-fluorocyclopropyl]-8-(methylamino)-6-{[2-(piperidin-4-ylmethoxy)pyridin-3-yl]amino}imidazo[1,2-b]pyridazine-3-carboxamide (Intermediate LO)

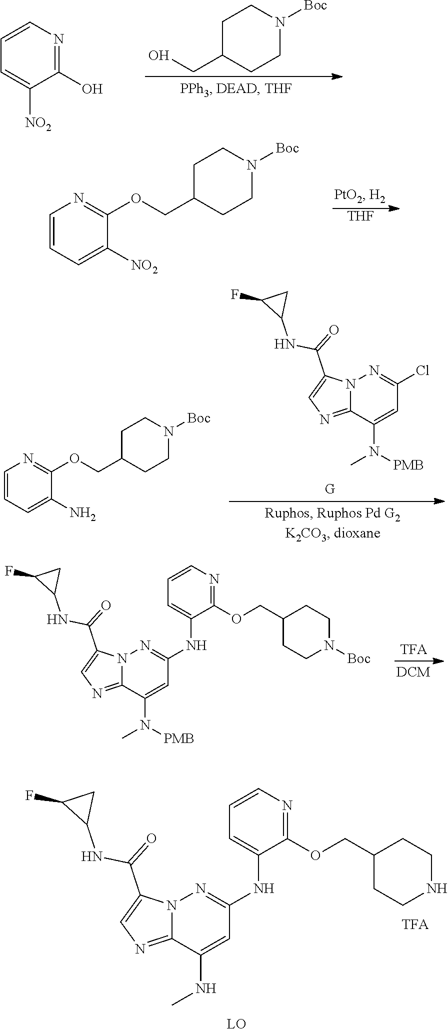

### Step 1—Tert-butyl 4-{[(3-nitropyridin-2-yl)oxy]methyl}piperidine-1-carboxylate

To a stirred solution of 3-nitropyridin-2-ol (10 g, 70 mmol, CAS #6332-56-5) and tert-butyl 4-(hydroxymethyl)piperidine-1-carboxylate (15.37 g, 71.38 mmol, CAS #123855-51-6) in THF (300 mL) were added PPh₃ (37.44 g, 142.8 mmol) and DEAD (24.86 g, 142.8 mmol) at 0° C. under nitrogen atmosphere. The resulting mixture was stirred for 2 h at rt under nitrogen atmosphere. On completion, the mixture was concentrated under reduced pressure. The residue was purified by silica gel column chromatography, eluted with PE/EA (2:1), to afford the title compound (17 g, 71% yield) as an off-white solid. LC/MS (ESI, m/z): [(M+H)]⁺=338.2.

### Step 2—Tert-butyl 4-{[(3-aminopyridin-2-yl)oxy]methyl}piperidine-1-carboxylate

To a solution of tert-butyl 4-{[(3-nitropyridin-2-yl)oxy]methyl}piperidine-1-carboxylate (5 g, 15 mmol) in THF (30 mL) was added PtO₂ (0.34 g, 1.5 mmol) under nitrogen atmosphere. The reaction system was degassed under vacuum and purged with H₂ several times, then it was hydrogenated under H₂ balloon (˜1 atm) at 25° C. for 4 h. After completion of the reaction, Pd/C was filtered off through celite and the filter cake was washed with MeOH (3×100 mL). The corresponding filtrate was concentrated under reduced pressure to provide the title compound (3.6 g, 79% yield) as an off-white solid. LC/MS (ESI, m/z): [(M+H)]⁺=308.10.

### Step 3—Tert-butyl 4-[({3-[(3-{[(2S)-2-fluorocyclopropyl]carbamoyl}-8-{[(4-methoxyphenyl)methyl](methyl)amino}imidazo[1,2-b]pyridazin-6-yl)amino]pyridin-2-yl}oxy)methyl]piperidine-1-carboxylate

To a stirred solution of tert-butyl 4-{[(3-aminopyridin-2-yl)oxy]methyl}piperidine-1-carboxylate (3.6 g, 12 mmol) and 6-chloro-N-[(2S)-2-fluorocyclopropyl]-8-{[(4-methoxyphenyl)methyl](methyl)amino}imidazo[1,2-b]pyridazine-3-carboxamide (4.73 g, 11.7 mmol, Intermediate G) in dioxane (30 mL) were added RuPhos (1.09 g, 2.34 mmol) and K₂CO₃ (4.86 g, 35.1 mmol) at rt under argon atmosphere. The resulting mixture was stirred for additional 5 min at rt. To the above mixture was added RuPhos-PdCl-2nd G (0.91 g, 1.171 mmol) in portions over 5 min at rt. The mixture was stirred for additional 30 min at rt. The resulting mixture was then stirred for 16 h at 100° C. under nitrogen atmosphere. On completion, the mixture was cooled to rt and concentrated under reduced pressure. The residue was purified by silica gel column chromatography, eluted with PE/EA (1:1), to afford the title compound (2.8 g, 35% yield) as a light brown solid. LC/MS (ESI, m/z): [(M+H)]⁺=675.3.

### Step 4—N-[(2S)-2-fluorocyclopropyl]-8-(methylamino)-6-{[2-(piperidin-4-ylmethoxy)pyridin-3-yl]amino}imidazo[1,2-b]pyridazine-3-carboxamide Trifluoroacetate

To a stirred solution of tert-butyl 4-[({3-[(3-{[(2S)-2-fluorocyclopropyl]carbamoyl}-8-{[(4-methoxyphenyl)methyl](methyl)amino}imidazo[1,2-b]pyridazin-6-yl)amino]pyridin-2-yl}oxy)methyl]piperidine-1-carboxylate (2.8 g, 4.2 mmol) in DCM (30 mL) was added TFA (10 mL) at 0° C. under nitrogen atmosphere. The resulting mixture was stirred for 2 h at rt under nitrogen atmosphere. On completion, the mixture was concentrated under reduced pressure. The residue was purified by trituration with Et₂O (300 mL) to afford the title compound (2.0 g, 87% yield) as a light brown solid. LC/MS (ESI, m/z): [(M+H)]⁺=455.3.

### Example 1.312. Preparation of Tert-butyl 4-(2,3-dihydro-1H-indol-4-yl)piperidine-1-carboxylate (Intermediate LP)

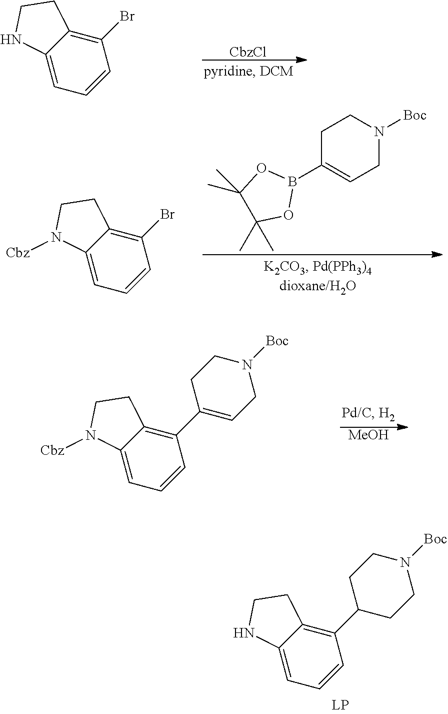

### Step 1—Benzyl 4-bromo-2,3-dihydroindole-1-carboxylate

To a mixture of 4-bromo-2,3-dihydro-1H-indole (5.00 g, 25.2 mmol) and pyridine (9.98 g, 126 mmol) in DCM (50 mL) was added benzyl chloroformate (21.53 g, 126.2 mmol) at 0° C. under nitrogen atmosphere. The resulting mixture was stirred for 2 h at rt under nitrogen atmosphere. On completion, the mixture was concentrated under reduced pressure. The residue was purified by silica gel column chromatography, eluted with PE/EA (5:1-1:5), to afford the title compound (7.00 g, 84% yield) as a colorless oil. LC/MS (ESI, m/z): [(M+H)]⁺=332.0.

### Step 2—Benzyl 4-[1-(tert-butoxycarbonyl)-3,6-dihydro-2H-pyridin-4-yl]-2,3-dihydroindole-1-carboxylate

To a stirred solution of benzyl 4-bromo-2,3-dihydroindole-1-carboxylate (8.00 g, 24.1 mmol) and tert-butyl 4-(4,4,5,5-tetramethyl-1,3,2-dioxaborolan-2-yl)-3,6-dihydro-2H-pyridine-1-carboxylate (7.45 g, 24.1 mmol) in dioxane (90 mL) and H₂O (18 mL) were added Pd(PPh₃)₄ (5.57 g, 4.82 mmol) and K₂CO₃ (6.66 g, 48.2 mmol) at rt under nitrogen atmosphere. The resulting mixture was stirred for 2 h at 80° C. under nitrogen atmosphere. On completion, the mixture was cooled to rt and concentrated under reduced pressure. The residue was purified by reverse flash chromatography (column, C18 silica gel; mobile phase A: water (10 mmol/L FA), mobile phase B: ACN, 50% to 70% gradient in 30 min; detector, UV 254 nm; the fractions were collected at 62%) and concentrated under reduced pressure to afford the title compound (6.25 g, 60% yield) as a black oil. LC/MS (ESI, m/z): [(M−56+H)]⁺=379.1.

### Step 3—Tert-butyl 4-(2,3-dihydro-1H-indol-4-yl)piperidine-1-carboxylate

To a stirred solution of benzyl 4-[1-(tert-butoxycarbonyl)-3,6-dihydro-2H-pyridin-4-yl]-2,3-dihydroindole-1-carboxylate (7.80 g, 18.0 mmol) in MeOH (10 mL) was added Pd/C (0.38 g, 3.6 mmol) at rt under nitrogen atmosphere. The resulting mixture was purged with hydrogen three times and stirred for 4 h at rt under hydrogen atmosphere. On completion, the mixture was filtered, and the filter cake was washed with MeOH (5×10 mL). The filtrate was concentrated under reduced pressure to afford the title compound (5.00 g, 92% yield) as a brown oil. LC/MS (ESI, m/z): [(M+H)]⁺=303.2.

### Example 1.313. Preparation of N-[(1R,2S)-2-fluorocyclopropyl]-8-(methylamino)-6-[4-(piperidin-4-yl)-2,3-dihydroindol-1-yl] imidazo [1,2-b]pyridazine-3-carboxamide (Intermediate LQ)

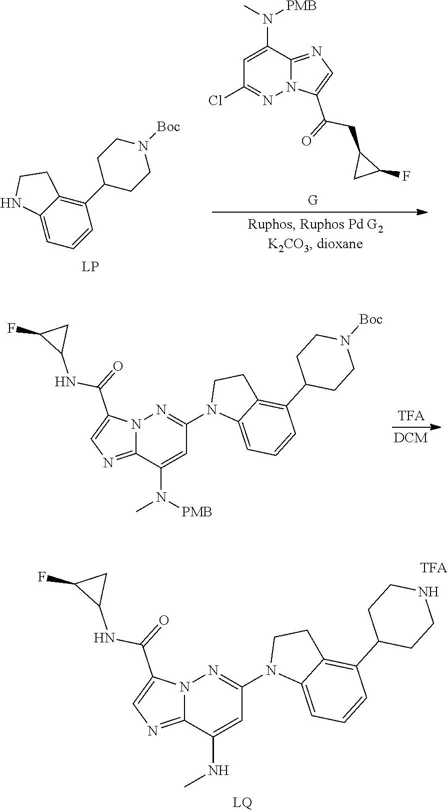

### Step 1—Tert-butyl 4-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]piperidine-1-carboxylate

To a stirred solution of tert-butyl 4-(2,3-dihydro-1H-indol-4-yl)piperidine-1-carboxylate (5.00 g, 16.5 mmol, Intermediate LP) and 6-chloro-N-[(1R,2S)-2-fluorocyclopropyl]-8-{[(4-methoxyphenyl)methyl](methyl)amino}imidazo[1,2-b]pyridazine-3-carboxamide (6.68 g, 16.5 mmol, Intermediate G) in dioxane (45 mL) were added K₂CO₃ (6.85 g, 50.0 mmol), RuPhos (1.54 g, 3.31 mmol) and RuPhos-PdCl-2nd G (1.29 g, 1.65 mmol) at rt under nitrogen atmosphere. The resulting mixture was stirred for 16 h at 100° C. under nitrogen atmosphere. On completion, the mixture was cooled to rt and concentrated under reduced pressure. The residue was purified by silica gel column chromatography, eluted with PE/EA (1:5), to afford the title compound (1.82 g, 16% yield) as a yellow solid. LC/MS (ESI, m/z): [(M+H)]⁺=670.3.

### Step 2—N-[(1R,2S)-2-fluorocyclopropyl]-8-(methylamino)-6-[4-(piperidin-4-yl)-2,3-dihydroindol-1-yl] imidazo[1,2-b]pyridazine-3-carboxamide Trifluoroacetate

To a stirred solution of tert-butyl 4-[1-(3-{[(1R,2S)-2-fluorocyclopropyl]carbamoyl}-8-{[(4-methoxyphenyl)methyl](methyl)amino}imidazo[1,2-b]pyridazin-6-yl)-2,3-dihydroindol-4-yl]piperidine-1-carboxylate (800 mg, 1 mmol) in DCM (8 mL) was added TFA (8 mL) at rt under nitrogen atmosphere. The resulting mixture was stirred for 1 h at rt under nitrogen atmosphere. On completion, the mixture was concentrated under reduced pressure. The residue was purified by trituration with Et₂O (10 mL) to afford the title compound (0.91 g) as a white solid. LC/MS (ESI, m/z): [(M+H)]⁺=450.2.

### Example 2. Method of Synthesis

### Example 2.1. Method 1: synthesis of N-{10-[1-(2,6-dioxopiperidin-3-yl)-3-methyl-2-oxo-1,3-benzodiazol-5-yl]decyl}-3-[(3-{[(1R,2S)-2-fluorocyclopropyl]carbamoyl}-8-(methylamino)imidazo[1,2-b]pyridazin-6-yl)amino]-2-oxo-[1,2′-bipyridine]-5′-carboxamide

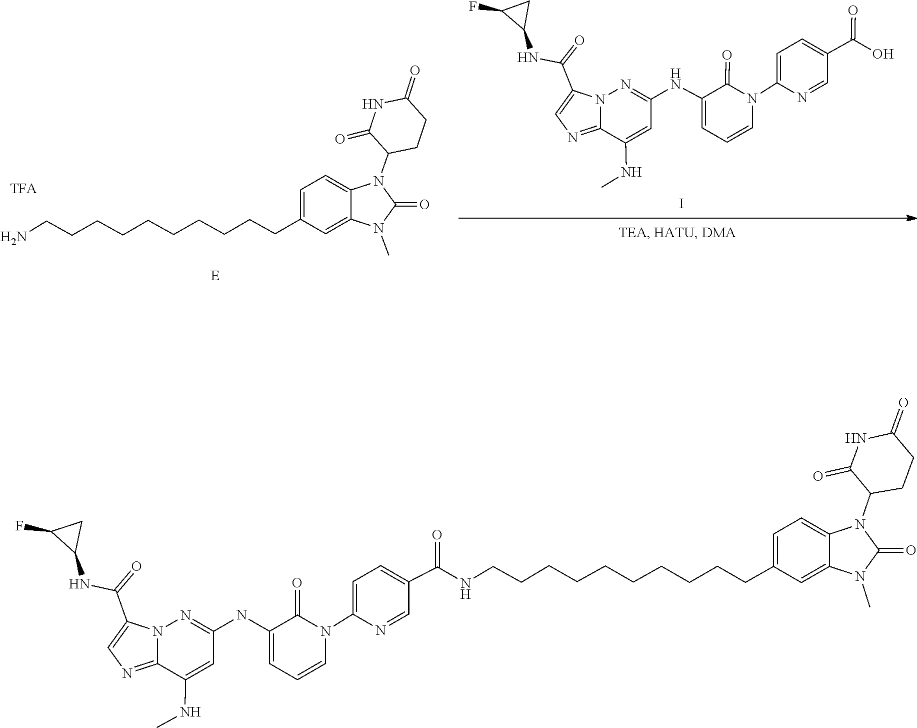

To a solution of 3-(5-(10-aminodecyl)-3-methyl-2-oxo-2,3-dihydro-1H-benzo[d]imidazol-1-yl)piperidine-2,6-dione trifluoroacetate (27.2 mg, 0.066 mmol, Intermediate E) and 3-[(3-{[(1R,2S)-2-fluorocyclopropyl]carbamoyl}-8-(methylamino)imidazo[1,2-b]pyridazin-6-yl)amino]-2-oxo-[1,2′-bipyridine]-5′-carboxylic acid (37.67 mg, 0.079 mmol, Intermediate I) in DMA (2 mL) were added TEA (0.03 mL, 0.2 mmol) and HATU (29.94 mg, 0.079 mmol) at rt. The resulting mixture was stirred for 1 h at rt. On completion, the residue was purified by reverse flash chromatography (column, silica gel; mobile phase A: water (0.1% FA), mobile phase B: MeCN, 20% to 40% gradient in 20 min; detector, UV 220/254 nm; the fractions were collected at 33% B) and concentrated under reduced pressure to afford the title compound (14.4 mg, 25% yield) as a white solid. ¹H NMR (400 MHz, DMSO-d₆) δ 11.09 (s, 1H), 9.04-8.99 (m, 1H), 8.81-8.74 (m, 1H), 8.69-8.63 (m, 2H), 8.42-8.35 (m, 1H), 8.04-7.94 (m, 2H), 7.92 (s, 1H), 7.61-7.55 (m, 1H), 7.55-7.47 (m, 1H), 7.04-6.96 (m, 2H), 6.88-6.82 (m, 1H), 6.44 (t, J=7.2 Hz, 1H), 6.39 (s, 1H), 5.37-5.28 (m, 1H), 4.99-4.76 (m, 1H), 3.33-3.27 (m, 4H), 3.04-2.96 (m, 1H), 2.89-2.84 (m, 4H), 2.69-2.55 (m, 4H), 2.05-1.95 (m, 1H), 1.61-1.49 (m, 4H), 1.41-1.18 (m, 14H), 1.06-0.91 (m, 1H); LC/MS (ESI, m/z): [(M+H)]⁺=875.2.

TABLE C
Compounds synthesized via Method 1, coupling the corresponding
amines and acids.

| | | | LCMS | |
|---|---|---|---|---|
| | | | (ESI+) | |
| Compound | | | m/z | |
| ID (K#) | Amine | Acid | (M + H)⁺ | ¹H NMR (400 MHZ, DMSO-d6) |
| K265 | DL | I | 833.1 | 11.07 (s, 1H), 9.02 (d, J = 2.3 Hz, 1H), 8.75 (t, J = |
| | | | | 5.5 Hz, 1H), 8.66 (s, 1H), 8.64 (d, J = 4.2 Hz, 1H), |
| | | | | 8.39 (dd, J = 8.5, 2.4 Hz, 1H), 8.02-7.96 (m, 2H), |
| | | | | 7.92 (s, 1H), 7.59 (dd, J = 7.1, 1.8 Hz, 1H), 7.50 (d, J = |
| | | | | 5.1 Hz, 1H), 6.99-6.93 (m, 2H), 6.90-6.85 (m, 1H), |
| | | | | 6.47-6.40 (m, 2H), 5.36 (dd, J = 12.5, 5.4 Hz, 1H), |
| | | | | 5.02-4.71 (m, 1H), 3.56 (s, 3H), 3.05-2.96 (m, 1H), |
| | | | | 2.93-2.89 (m, 2H), 2.88 (d, J = 4.9 Hz, 3H), 2.76- |
| | | | | 2.67 (m, 1H), 2.66-2.55 (m, 2H), 2.47-2.30 (m, 2H), |
| | | | | 2.05-1.96 (m, 1H), 1.66-1.53 (m, 4H), 1.42-1.36 (m, |
| | | | | 5H), 1.31-1.18 (m, 2H), 1.06-0.91 (m, 1H) |
| K264 | AQ | I | 791.4 | 11.07 (s, 1H), 9.02 (d, J = 2.4 Hz, 1H), 8.80-8.74 (m, |
| | | | | 1H), 8.71-8.61 (m, 2H), 8.41-8.36 (m, 1H), 8.03-7.93 |
| | | | | (m, 3H), 7.60-7.57 (m, 1H), 7.53-7.45 (m, 1H), 7.06 |
| | | | | (s, 1H), 7.03-6.98 (m, 1H), 6.92-6.87 (m, 1H), 6.46- |
| | | | | 6.40 (m, 2H), 5.34 (dd, J = 12.7, 5.3 Hz, 1H), 4.99- |
| | | | | 4.76 (m, 1H), 3.40-3.34 (m, 4H), 3.04-2.96 (m, 1H), |
| | | | | 2.95-2.84 (m, 4H), 2.77-2.58 (m, 2H), 2.49-2.44 (m, |
| | | | | 3H), 2.05-1.95 (m, 1H), 1.72-1.53 (m, 4H), 1.32-1.17 |
| | | | | (m, 1H), 1.06-0.92 (m, 1H) |
| K266 | EH | I | 861.4 | 11.09 (s, 1H), 9.02 (s, 1H), 8.81-8.73 (m, 1H), 8.70- |
| | | | | 8.63 (m, 2H), 8.38 (d, J = 8.1 Hz, 1H), 8.05-7.94 (m, |
| | | | | 2H), 7.92 (s, 1H), 7.60-7.49 (m, 2H), 6.99-6.91 (m, |
| | | | | 2H), 6.89-6.82 (m, 1H), 6.47-6.37 (m, 2H), 5.42-5.29 |
| | | | | (m, 1H), 5.01-4.75 (m, 1H), 3.55 (s, 3H), 3.52-3.47 |
| | | | | (m, 2H), 3.25-3.06 (m, 2H), 3.04-2.96 (m, 1H), 2.94- |
| | | | | 2.82 (m, 4H), 2.76-2.59 (m, 2H), 2.07-1.91 (m, 1H), |
| | | | | 1.68-1.49 (m, 4H), 1.46-1.13 (m, 11H), 1.07-0.90 (m, |
| | | | | 1H) |
| K259 | N | I | 791.4 | 11.10 (s, 1H), 9.03-9.00 (m, 1H), 8.89-8.78 (m, 1H), |
| | | | | 8.68 (s, 1H), 8.65 (d, J = 4.3 Hz, 1H), 8.39 (dd, J = |
| | | | | 8.5, 2.4 Hz, 1H), 8.04-7.96 (m, 2H), 7.92-7.92 (m, |
| | | | | 1H), 7.63-7.56 (m, 1H), 7.53-7.49 (m, 1H), 6.99-6.95 |
| | | | | (m, 2H), 6.93-6.89 (m, 1H), 6.54-6.37 (m, 2H), 5.37 |
| | | | | (dd, J = 12.8, 5.3 Hz, 1H), 4.98-4.78 (m, 1H), 3.57 |
| | | | | (s, 3H), 3.42-3.37 (m, 2H), 3.03-2.93 (m, 3H), 2.91- |
| | | | | 2.85 (m, 4H), 2.78-2.55 (m, 2H), 2.05-1.97 (m, 1H), |
| | | | | 1.74-1.63 (m, 4H), 1.34-1.13 (m, 1H), 1.05-0.95 (m, |
| | | | | 1H) |
| K260 | DJ | I | 833.2 | 11.09 (s, 1H), 9.02 (d, J = 2.3 Hz, 1H), 8.77 (t, J = |
| | | | | 5.6 Hz, 1H), 8.68 (s, 1H), 8.65 (d, J = 4.2 Hz, 1H), |
| | | | | 8.38 (dd, J = 8.4, 2.4 Hz, 1H), 8.04-7.95 (m, 2H), |
| | | | | 7.94-7.92 (m, 1H), 7.58 (dd, J = 7.1, 1.7 Hz, 1H), |
| | | | | 7.54-7.50 (m, 1H), 7.04-6.98 (m, 2H), 6.89-6.84 (m, |
| | | | | 1H), 6.47-6.41 (m, 2H), 5.34 (dd, J = 12.7, 5.4 Hz, |
| | | | | 1H), 5.00-4.78 (m, 1H), 3.32 (s, 3H), 3.04-2.96 (m, |
| | | | | 1H), 2.95-2.89 (m, 1H), 2.87 (d, J = 4.5 Hz, 3H), |
| | | | | 2.76-2.67 (m, 1H), 2.66-2.58 (m, 3H), 2.43-2.30 (m, |
| | | | | 2H), 2.06-1.95 (m, 1H), 1.64-1.51 (m, 4H), 1.37-1.32 |
| | | | | (m, 4H), 1.29-1.19 (m, 3H), 1.08-0.93 (m, 1H) |
| K262 | DP | I | 851.5 | 11.10 (s, 1H), 9.04 (d, J = 2.4 Hz, 1H), 8.89 (t, J = |
| | | | | 5.6 Hz, 1H), 8.69-8.62 (m, 2H), 8.40 (dd, J = 8.4, 2.3 |
| | | | | Hz, 1H), 8.03-7.95 (m, 2H), 7.92 (s, 1H), 7.60-7.49 |
| | | | | (m, 2H), 7.07 (s, 1H), 7.00 (d, J = 8.0 Hz, 1H), 6.90 |
| | | | | (d, J = 8.0 Hz, 1H), 6.47-6.37 (m, 2H), 5.34 (dd, J = |
| | | | | 12.7, 5.3 Hz, 1H), 4.98-4.74 (m, 1H), 3.65-3.56 (m, |
| | | | | 3H), 3.50-3.37 (m, 6H), 3.32 (s, 3H), 3.00 (s, 1H), |
| | | | | 2.91-2.78 (m, 6H), 2.77-2.57 (m, 2H), 2.07-1.92 (m, |
| | | | | 1H), 1.30-1.19 (m, 2H), 1.05-0.91 (m, 1H) |
| K263 | AM | I | 939.4 | 11.09 (s, 1H), 9.03 (d, J = 2.3 Hz, 1H), 8.92-8.85 (m, |
| | | | | 1H), 8.67 (s, 1H), 8.65 (d, J = 4.2 Hz, 1H), 8.44-8.36 |
| | | | | (m, 1H), 8.04-7.95 (m, 2H), 7.92 (s, 1H), 7.62-7.55 |
| | | | | (m, 1H), 7.51 (q, J = 5.0 Hz, 1H), 7.07 (d, J = 1.5 Hz, |
| | | | | 1H), 7.00 (d, J = 8.1 Hz, 1H), 6.93-6.86 (m, 1H), |
| | | | | 6.43 (t, J = 7.2 Hz, 1H), 6.40 (s, 1H), 5.38-5.29 (m, |
| | | | | 1H), 4.99-4.77 (m, 1H), 3.64-3.52 (m, 8H), 3.52-3.43 |
| | | | | (m, 10H), 3.31 (s, 3H), 3.04-2.96 (m, 1H), 2.96-2.77 |
| | | | | (m, 6H), 2.77-2.56 (m, 2H), 2.05-1.95 (m, 1H), 1.32- |
| | | | | 1.18 (m, 1H), 1.06-0.91 (m, 1H) |
| K369 | DN | I | 849.7 | 11.09 (d, J = 10.0 Hz, 1H), 8.70-8.62 (m, 2H), 8.09 |
| | | | | (t, J = 10.2 Hz, 1H), 8.02-7.91 (m, 3H), 7.60-7.48 |
| | | | | (m, 2H), 7.32-7.15 (m, 1H), 7.06-6.85 (m, 3H), 6.46- |
| | | | | 6.31 (m, 2H), 5.45-5.28 (m, 1H), 5.03-4.74 (m, 1H), |
| | | | | 3.58 (s, 3H), 3.50-3.48 (m, 4H), 3.32 (s, 3H), 3.04- |
| | | | | 2.97 (m, 2H), 2.87 (d, J = 4.8 Hz, 3H), 2.83-2.76 (m, |
| | | | | 1H), 2.70-2.54 (m, 4H), 2.03-1.63 (m, 6H), 1.28-1.18 |
| | | | | (m, 1H), 1.05-0.89 (m, 1H) |
| K219 | AQ | BS | 790.2 | 11.08 (s, 1H), 8.69-8.62 (m, 2H), 8.59 (t, J = 5.6 Hz, |
| | | | | 1H), 8.01 (dd, J = 7.4, 1.8 Hz, 1H), 7.97-7.93 (m, |
| | | | | 3H), 7.70-7.60 (m, 2H), 7.54-7.47 (m, 1H), 7.36 (dd, |
| | | | | J = 6.9, 1.7 Hz, 1H), 7.07-7.03 (m, 1H), 7.00 (d, J = |
| | | | | 8.0 Hz, 1H), 6.92-6.85 (m, 1H), 6.43-6.35 (m, 2H), |
| | | | | 5.33 (dd, J = 12.7, 5.4 Hz, 1H), 4.97-4.80 (m, 1H), |
| | | | | 3.35-3.29 (m, 5H), 3.04-2.97 (m, 1H), 2.91-2.84 (m, |
| | | | | 4H), 2.76-2.70 (m, 1H), 2.69-2.62 (m, 3H), 2.04-1.95 |
| | | | | (m, 1H), 1.70-1.61 (m, 2H), 1.60-1.51 (m, 2H), 1.31- |
| | | | | 1.22 (m, 1H), 1.06-0.95 (m, 1H) |
| K223 | CL | BS | 846.4 | 11.08 (s, 1H), 8.66 (d, J = 4.2 Hz, 1H), 8.63 (s, 1H), |
| | | | | 8.59-8.53 (m, 1H), 8.01 (dd, J = 7.4, 1.7 Hz, 1H), |
| | | | | 7.98-7.92 (m, 3H), 7.71-7.61 (m, 2H), 7.54-7.47 (m, |
| | | | | 1H), 7.35 (dd, J = 6.9, 1.7 Hz, 1H), 7.04-6.95 (m, |
| | | | | 2H), 6.85 (dd, J = 8.1, 1.6 Hz, 1H), 6.42-6.37 (m, |
| | | | | 2H), 5.33 (dd, J = 12.7, 5.3 Hz, 1H), 5.02-4.76 (m, |
| | | | | 1H), 3.31 (s, 3H), 3.30-3.20(m, 2H), 3.05-2.97 (m, |
| | | | | 1H), 2.96-2.82 (m, 4H), 2.76-2.56 (m, 4H), 2.06-1.91 |
| | | | | (m, 1H), 1.63-1.42 (m, 4H), 1.36-1.20 (m, 9H), 1.11- |
| | | | | 0.90 (m, 1H) |
| K224 | CI | BS | 846.4 | 11.09 (s, 1H), 8.67-8.64 (m, 1H), 8.62 (s, 1H), 8.59- |
| | | | | 8.54 (m, 1H), 8.02-7.99 (m, 1H), 7.97-7.94 (m, 2H), |
| | | | | 7.93 (s, 1H), 7.70-7.61 (m, 2H), 7.53-7.47 (m, 1H), |
| | | | | 7.37-7.33 (m, 1H), 6.97-6.92 (m, 2H), 6.88-6.82 (m, |
| | | | | 1H), 6.42-6.36 (m, 2H), 5.36 (dd, J = 12.4, 5.4 Hz, |
| | | | | 1H), 5.01-4.76 (m, 1H), 3.54 (s, 3H), 3.30-3.23 (m, |
| | | | | 2H), 3.05-2.97 (m, 1H), 2.91-2.83 (m, 6H), 2.75-2.57 |
| | | | | (m, 2H), 2.04-1.95 (m, 1H), 1.63-1.48 (m, 4H), 1.44- |
| | | | | 1.13 (m, 9H), 1.09-0.92 (m, 1H) |
| K225 | E | BS | 874.3 | 11.08 (s, 1H), 8.65 (d, J = 4.2 Hz, 1H), 8.62 (s, 1H), |
| | | | | 8.56 (t, J = 5.6 Hz, 1H), 8.02-7.99 (m, 1H), 7.97-7.93 |
| | | | | (m, 2H), 7.92 (s, 1H), 7.71-7.60 (m, 2H), 7.54-7.46 |
| | | | | (m, 1H), 7.35 (dd, J = 6.9, 1.7 Hz, 1H), 7.05-6.93 (m, |
| | | | | 2H), 6.88-6.81 (m, 1H), 6.43-6.36 (m, 2H), 5.33 (dd, |
| | | | | J = 12.8, 5.4 Hz, 1H), 5.00-4.76 (m, 1H), 3.31 (s, |
| | | | | 3H), 3.30-3.23 (m, 2H), 3.04-2.96 (m, 1H), 2.95-2.88 |
| | | | | (m, 1H), 2.87 (d, J = 4.5 Hz, 3H), 2.77-2.63 (m, 2H), |
| | | | | 2.62-2.55 (m, 2H), 2.05-1.93 (m, 1H), 1.63-1.45 (m, |
| | | | | 4H), 1.36-1.21 (m, 13H), 1.08-0.92 (m, 1H) |
| K226 | EL | I | 874.5 | 11.08 (s, 1H), 8.67-8.64 (m, 1H), 8.62 (s, 1H), 8.58- |
| | | | | 8.54 (m, 1H), 8.04-7.98 (m, 1H), 7.98-7.91 (m, 3H), |
| | | | | 7.69-7.58 (m, 2H), 7.53-7.46 (m, 1H), 7.40-7.33 (m, |
| | | | | 1H), 7.02-6.91 (m, 2H), 6.88-6.81 (m, 1H), 6.43-6.34 |
| | | | | (m, 2H), 5.36 (dd, J = 12.6, 5.3 Hz, 1H), 5.01-4.78 |
| | | | | (m, 1H), 3.54 (s, 3H), 3.32-3.22 (m, 2H), 3.04-2.96 |
| | | | | (m, 1H), 2.93-2.83 (m, 6H), 2.77-2.57 (m, 2H), 2.05- |
| | | | | 1.94 (m, 1H), 1.68-1.46 (m, 4H), 1.43-1.19 (m, 13H), |
| | | | | 1.09-0.94 (d, J = 25.5 Hz, 1H) |
| K228 | N | I | 791.5 | 11.09 (s, 1H), 8.87 (t, J = 5.6 Hz, 1H), 8.77 (d, J = |
| | | | | 5.1 Hz, 1H), 8.69 (s, 1H), 8.66 (d, J = 4.2 Hz, 1H), |
| | | | | 8.25-8.22 (m, 1H), 8.03 (dd, J = 7.4, 1.8 Hz, 1H), |
| | | | | 7.93 (s, 1H), 7.89 (dd, J = 5.1, 1.5 Hz, 1H), 7.57 (dd, |
| | | | | J = 7.1, 1.7 Hz, 1H), 7.54-7.49 (m, 1H), 6.99-6.93 |
| | | | | (m, 2H), 6.92-6.86 (m, 1H), 6.46-6.43 (m, 1H), 6.42- |
| | | | | 6.40 (m, 1H), 5.36 (dd, J = 12.6, 5.4 Hz, 1H), 5.01- |
| | | | | 4.76 (m, 1H), 3.46 (s, 3H), 3.41-3.33 (m, 2H), 3.05- |
| | | | | 2.98 (m, 1H), 2.97-2.89 (m, 2H), 2.88-2.83 (m, 4H), |
| | | | | 2.77-2.57 (m, 2H), 2.05-1.94 (m, 1H), 1.73-1.61 (m, |
| | | | | 4H), 1.32-1.18 (m, 1H), 1.06-0.93 (m, 1H) |
| K229 | BT | P | 819.4 | 11.08 (s, 1H), 8.88-8.81 (m, 1H), 8.77 (d, J = 5.1 Hz, |
| | | | | 1H), 8.71-8.63 (m, 2H), 8.23 (s, 1H), 8.02 (dd, J = |
| | | | | 7.3, 1.9 Hz, 1H), 7.92 (s, 1H), 7.89 (dd, J = 5.2, 1.5 |
| | | | | Hz, 1H), 7.59-7.48 (m, 2H), 7.05-6.93 (m, 2H), 6.86 |
| | | | | (d, J = 8.0 Hz, 1H), 6.47-6.38 (m, 2H), 5.33 (dd, J = |
| | | | | 12.8, 5.4 Hz, 1H), 5.02-4.75 (m, 1H), 3.32 (s, 3H), |
| | | | | 3.31-3.26 (m, 2H), 3.04-2.95 (m, 1H), 2.94-2.89 (m, |
| | | | | 1H), 2.87 (d, J = 4.9 Hz, 3H), 2.73-2.66 (m, 1H), |
| | | | | 2.65-2.57 (m, 3H), 2.04-1.93 (m, 1H), 1.67-1.47 (m, |
| | | | | 4H), 1.40-1.30 (m, 4H), 1.29-1.19 (m, 1H), 1.05-0.94 |
| | | | | (d, J = 24.9 Hz, 1H) |
| K230 | BV | P | 819.5 | 11.09 (s, 1H), 8.87-8.82 (m, 1H), 8.77 (d, J = 5.1 Hz, |
| | | | | 1H), 8.71 (s, 1H), 8.68-8.65 (m, 1H), 8.23 (s, 1H), |
| | | | | 8.03 (dd, J = 7.4, 1.8 Hz, 1H), 7.95 (s, 1H), 7.89 (dd, |
| | | | | J = 5.2, 1.5 Hz, 1H), 7.57 (dd, J = 7.1, 1.7 Hz, 1H), |
| | | | | 7.53-7.48 (m, 1H), 6.98-6.92 (m, 2H), 6.88-6.84 (m, |
| | | | | 1H), 6.48-6.38 (m, 2H), 5.36 (dd, J = 12.7, 5.4 Hz, |
| | | | | 1H), 4.99-4.75 (m, 1H), 3.55 (s, 3H), 3.36-3.25 (m, |
| | | | | 2H), 3.04-2.95 (m, 1H), 2.93-2.82 (m, 4H), 2.76-2.57 |
| | | | | (m, 2H), 2.55-2.52 (m, 2H), 2.03-1.96 (m, 1H), 1.67- |
| | | | | 1.51 (m, 4H), 1.47-1.34 (m, 4H), 1.30-1.17 (m, 1H), |
| | | | | 1.07-0.92 (m, 1H) |
| K231 | CL | I | 847.4 | 11.08 (s, 1H), 8.83 (t, J = 5.6 Hz, 1H), 8.77 (d, J = |
| | | | | 5.1 Hz, 1H), 8.69 (s, 1H), 8.66 (d, J = 4.2 Hz, 1H), |
| | | | | 8.24-8.22 (m, 1H), 8.03 (dd, J = 7.3, 1.8 Hz, 1H), |
| | | | | 7.95 (s, 1H), 7.89 (dd, J = 5.2, 1.5 Hz, 1H), 7.56 (dd, |
| | | | | J = 7.1, 1.7 Hz, 1H), 7.53-7.47 (m, 1H), 7.03-6.96 |
| | | | | (m, 2H), 6.87-6.82 (m, 1H), 6.46-6.43 (d, J = 7.2 Hz, |
| | | | | 1H), 6.42-6.40 (m, 1H), 5.33 (dd, J = 12.7, 5.4 Hz, |
| | | | | 1H), 5.00-4.77 (m, 1H), 3.32 (s, 3H), 3.30-3.25 (m, |
| | | | | 2H), 3.04-2.97 (m, 1H), 2.91-2.83 (m, 4H), 2.78-2.56 |
| | | | | (m, 4H), 2.06-1.96 (m, 1H), 1.64-1.48 (m, 4H), 1.38- |
| | | | | 1.27 (m, 8H), 1.26-1.18 (m, 1H), 1.06-1.93 (m, 1H) |
| K232 | CI | I | 847.5 | 11.09 (s, 1H), 8.84 (t, J = 5.6 Hz, 1H), 8.77 (d, J = |
| | | | | 5.2 Hz, 1H), 8.71 (s, 1H), 8.66 (d, J = 4.2 Hz, 1H), |
| | | | | 8.24-8.21 (m, 1H), 8.03 (dd, J = 7.4, 1.8 Hz, 1H), |
| | | | | 7.96 (s, 1H), 7.89 (dd, J = 5.1, 1.5 Hz, 1H), 7.57 (dd, |
| | | | | J = 7.1, 1.7 Hz, 1H), 7.52-7.46 (m, 1H), 6.98-6.90 |
| | | | | (m, 2H), 6.88-6.82 (m, 1H), 6.48-6.39 (m, 2H), 5.36 |
| | | | | (dd, J = 12.6, 5.4 Hz, 1H), 5.00-4.74 (m, 1H), 3.54 |
| | | | | (s, 3H), 3.33-3.23 (m, 2H), 3.04-2.95 (m, 1H), 2.93- |
| | | | | 2.81 (m, 6H), 2.77-2.56 (m, 2H), 2.08-1.90 (m, 1H), |
| | | | | 1.67-1.47 (m, 4H), 1.41-1.30 (m, 8H), 1.29-1.18 (m, |
| | | | | 1H), 1.06-0.93 (m, 1H) |
| K233 | E | P | 875.2 | 11.07 (s, 1H), 8.83 (t, J = 5.6 Hz, 1H), 8.77 (d, J = |
| | | | | 5.2 Hz, 1H), 8.67 (s, 1H), 8.65 (d, J = 4.3 Hz, 1H), |
| | | | | 8.23 (t, J = 1.0 Hz, 1H), 8.02 (dd, J = 7.4, 1.7 Hz, |
| | | | | 1H), 7.92 (s, 1H), 7.89 (dd, J = 5.2, 1.5 Hz, 1H), 7.56 |
| | | | | (dd, J = 7.1, 1.7 Hz, 1H), 7.53-7.49 (m, 1H), 7.01 (d, |
| | | | | J = 1.5 Hz, 1H), 6.98 (d, J = 8.0 Hz, 1H), 6.85 (dd, J = |
| | | | | 8.0, 1.6 Hz, 1H), 6.44 (t, J = 7.2 Hz, 1H), 6.39 (s, |
| | | | | 1H), 5.33 (dd, J = 12.7, 5.4 Hz, 1H), 4.98-4.78 (m, |
| | | | | 1H), 3.33-3.27 (m, 4H), 3.04-2.97 (m, 1H), 2.95-2.85 |
| | | | | (m, 4H), 2.78-2.54 (m, 5H), 2.09-1.91 (m, 1H), 1.62- |
| | | | | 1.52 (m, 4H), 1.41-1.16 (m, 13H), 1.05-0.94 (m, 1H) |
| K234 | EL | P | 875.4 | 11.08 (s, 1H), 8.86-8.80 (m, 1H), 8.77 (d, J = 5.1 Hz, |
| | | | | 1H), 8.68 (s, 1H), 8.67-8.64 (m, 1H), 8.23 (s, 1H), |
| | | | | 8.04-7.99 (m, 1H), 7.92 (s, 1H), 7.89 (dd, J = 5.1, 1.5 |
| | | | | Hz, 1H), 7.56 (dd, J = 7.1, 1.7 Hz, 1H), 7.53-7.47 (m, |
| | | | | 1H), 6.98-6.91 (m, 2H), 6.87-6.82 (m, 1H), 6.47-6.41 |
| | | | | (m, 1H), 6.40 (s, 1H), 5.36 (dd, J = 12.6, 5.4 Hz, 1H), |
| | | | | 4.99-4.77 (m, 1H), 3.54 (s, 3H), 3.32-3.25 (m, 2H), |
| | | | | 3.05-2.96 (m, 1H), 2.87 (d, J = 4.4 Hz, 6H), 2.76- |
| | | | | 2.59 (m, 2H), 2.04-1.94 (s, 1H), 1.63-1.48 (m, 4H), |
| | | | | 1.42-1.18 (m, 13H), 1.05-0.93 (m, 1H) |
| K235 | DP | BS | 850.4 | 11.07 (s, 1H), 8.67-8.62 (m, 2H), 8.60 (s, 1H), 8.02- |
| | | | | 7.94 (m, 3H), 7.92 (s, 1H), 7.71-7.60 (m, 2H), 7.53- |
| | | | | 7.46 (m, 1H), 7.36-7.31 (m, 1H), 7.06 (s, 1H), 7.01- |
| | | | | 6.96 (m, 1H), 6.92-6.86 (m, 1H), 6.42-6.35 (m, 2H), |
| | | | | 5.33 (dd, J = 12.5, 5.4 Hz, 1H), 5.02-4.78 (m, 1H), |
| | | | | 3.64-3.58 (m, 2H), 3.58-3.50 (m, 6H), 3.48-3.41 (m, |
| | | | | 2H), 3.31 (s, 3H),3.05-2.96 (m, 1H), 2.96-2.86 (m, |
| | | | | 1H), 2.87 (d, J = 4.8 Hz, 3H), 2.84-2.78 (m, 2H), |
| | | | | 2.75-2.58 (m, 2H), 2.04-1.95 (m, 1H), 1.33-1.19 (m, |
| | | | | 1H), 1.09-0.94 (m, 1H) |
| K275 | AN | BE | 828.4 | 11.09 (s, 1H), 8.62 (d, J = 4.3 Hz, 1H), 8.29 (s, 1H), |
| | | | | 7.99 (d, J = 7.5 Hz, 1H), 7.89 (dd, J = 5.0, 1.7 Hz, |
| | | | | 1H), 7.85 (s, 1H), 7.50 (q, J = 4.9 Hz, 1H), 7.06 (s, |
| | | | | 1H), 7.01-6.97 (m, 2H), 6.91-6.87 (m, 1H), 6.04 (s, |
| | | | | 1H), 5.38-5.27 (m, 2H), 4.87-4.55 (m, 1H), 4.13 (s, |
| | | | | 2H), 3.74-3.70 (m, 1H), 3.65-3.57 (m, 4H), 3.56-3.52 |
| | | | | (m, 4H), 3.30 (s, 3H), 2.95-2.77 (m, 8H), 2.76-2.56 |
| | | | | (m, 2H), 2.10-1.82 (m, 3H), 1.80-1.54 (m, 2H), 1.20- |
| | | | | 1.00 (m, 1H), 0.66-0.56 (m, 1H) |
| K276 | AN | BI | 916.4 | 11.07 (s, 1H), 8.62 (d, J = 4.2 Hz, 1H), 8.27 (s, 1H), |
| | | | | 7.98 (dd, J = 7.7, 1.8 Hz, 1H), 7.88 (dd, J = 4.9, 1.7 |
| | | | | Hz, 1H), 7.86 (s, 1H), 7.47 (d, J = 5.1 Hz, 1H), 7.05 |
| | | | | (d, J = 1.5 Hz, 1H), 7.00-6.95 (m, 2H), 6.89 (dd, J = |
| | | | | 8.1, 1.6 Hz, 1H), 6.05 (s, 1H), 5.35-5.29 (m, 2H), |
| | | | | 4.83-4.64 (m, 1H), 4.12 (s, 2H), 3.74-3.67 (m, 2H), |
| | | | | 3.58 (t, J = 7.0 Hz, 6H), 3.54-3.47 (m, 11H), 3.32- |
| | | | | 3.26 (m, 4H), 2.89 (d, J = 4.9 Hz, 3H), 2.87-2.79 (m, |
| | | | | 2H), 2.76-2.55 (m, 2H), 2.04-1.82 (m, 3H), 1.70-1.61 |
| | | | | (m, 2H), 1.13-1.07 (m, 1H), 0.606-0.55 (m, 1H) |
| K331 | CS | I | 841.4 | 11.12 (s, 1H), 8.69-8.62 (m, 3H), 8.11-8.03 (m, 1H), |
| | | | | 8.03-7.90 (m, 3H), 7.59 (d, J = 7.0 Hz, 1H), 7.50 (d, |
| | | | | J = 5.8 Hz, 1H), 7.12 (d, J = 7.8 Hz, 1H), 7.08 (d, J = |
| | | | | 7.8 Hz, 1H), 7.00 (t, J = 7.8 Hz, 1H), 6.46-6.34 (m, |
| | | | | 2H), 5.39 (dd, J = 12.7, 5.4 Hz, 1H), 5.00-4.76 (m, |
| | | | | 1H), 4.61-4.47 (m, 1H), 3.72-3.67 (m, 1H), 3.65 (s, |
| | | | | 3H), 3.25-3.12 (m, 1H), 3.04-2.96 (m, 1H), 2.95-2.91 |
| | | | | (m, 1H), 2.88 (d, J = 4.80 Hz, 3H), 2.78-2.59 (m, |
| | | | | 3H), 2.11-1.72 (m, 5H), 1.41-1.17 (m, 4H), 1.05-0.91 |
| | | | | (m, 1H) |
| K215 | AP | I | 1027.5 | 11.07 (s, 1H), 9.04 (d, J = 2.2 Hz, 1H), 8.93-8.85 (m, |
| | | | | 1H), 8.68-8.62 (m, 2H), 8.40 (dd, J = 8.4, 2.4 Hz, |
| | | | | 1H), 8.04-7.96 (m, 2H), 7.92 (s, 1H), 7.59 (dd, J = |
| | | | | 7.1, 1.7 Hz, 1H), 7.52-7.46 (m, 1H), 7.06 (s, 1H), |
| | | | | 7.00 (d, J = 8.0 Hz, 1H), 6.93-6.86 (m, 1H), 6.44 (t, J = |
| | | | | 7.2 Hz, 1H), 6.39 (t, J = 1.8 Hz, 1H), 5.34 (dd, J = |
| | | | | 12.7, 5.3 Hz, 1H), 5.00-4.76 (m, 1H), 3.68-3.53 (m, |
| | | | | 10H), 3.53-3.45 (m, 12H), 3.43-3.39 (m, 4H), 3.32 |
| | | | | (s, 3H), 3.08-2.97 (m, 1H), 2.95-2.79 (m, 6H), 2.77- |
| | | | | 2.58 (m, 2H), 2.06-1.96 (m, 1H), 1.32-1.18 (m, 1H), |
| | | | | 1.07-0.92 (m, 1H) |
| K216 | EI | I | 851.3 | 11.10 (s, 1H), 9.04 (d, J = 2.3 Hz, 1H), 8.87 (t, J = |
| | | | | 5.4 Hz, 1H), 8.64 (d, J = 3.9 Hz, 2H), 8.40 (dd, J = |
| | | | | 8.5, 2.3 Hz, 1H), 7.99 (d, J = 8.0 Hz, 2H), 7.93 (s, |
| | | | | 1H), 7.58 (d, J = 7.0 Hz, 1H), 7.53-7.45 (m, 1H), |
| | | | | 7.01-6.92 (m, 2H), 6.92-6.87 (m, 1H), 6.43 (t, J = 7.2 |
| | | | | Hz, 1H), 6.38 (d, J = 2.9 Hz, 1H), 5.37 (dd, J = 12.4, |
| | | | | 5.3 Hz, 1H), 5.00-4.75 (m, 1H), 3.68 (t, J = 6.8 Hz, |
| | | | | 2H), 3.63-3.55 (m, 7H), 3.49-3.42 (m, 4H), 3.14 (t, J = |
| | | | | 6.9 Hz, 2H), 3.06-2.97 (m, 1H), 2.96-2.83 (m, 4H), |
| | | | | 2.78-2.58 (m, 2H), 2.05-1.95 (m, 1H), 1.32-1.18 (m, |
| | | | | 1H), 1.07-0.92 (m, 1H) |
| K217 | EG | I | 1027.5 | 11.08 (s, 1H), 9.06-9.01 (m, 1H), 8.91-8.83 (m, 1H), |
| | | | | 8.69-8.60 (m, 2H), 8.44-8.37 (m, 1H), 8.02-7.93 (m, |
| | | | | 2H), 7.62-7.56 (m, 1H), 7.53-7.47 (m, 1H), 7.30-7.09 |
| | | | | (m, 1H), 7.04-6.87 (m, 3H), 6.48-6.38 (m, 2H), 5.41- |
| | | | | 5.32 (m, 1H), 5.01-4.67 (m, 1H), 3.75-3.62 (m, 5H), |
| | | | | 3.62-3.54 (m, 9H), 3.54-3.49 (m, 9H), 3.48-3.46 (m, |
| | | | | 6H), 3.18-3.10 (m, 2H), 3.04-2.96 (m, 1H), 2.96-2.83 |
| | | | | (m, 4H), 2.77-2.55 (m, 2H), 2.04-1.94 (m, 1H), 1.32- |
| | | | | 1.18 (m, 1H), 1.07-0.92 (m, 1H) |
| K218 | EJ | I | 939.4 | 10.97 (s, 1H), 9.04 (d, J = 2.2 Hz, 1H), 8.87 (t, J = |
| | | | | 5.5 Hz, 1H), 8.68-8.61 (m, 2H), 8.40 (dd, J = 8.5, 2.4 |
| | | | | Hz, 1H), 8.03-7.95 (m, 2H), 7.92 (s, 1H), 7.58 (dd, |
| | | | | J = 7.1, 1.7 Hz, 1H), 7.53-7.45 (m, 1H), 7.01-6.94 (m, |
| | | | | 2H), 6.94-6.87 (m, 1H), 6.47-6.37 (m, 2H), 5.36 (dd, |
| | | | | J = 12.5, 5.3 Hz, 1H), 5.00-4.76 (m, 1H), 3.66 (t, J = |
| | | | | 6.9 Hz, 2H), 3.60-3.52 (m, 8H), 3.52-3.45 (m, 8H), |
| | | | | 3.45-3.29 (m, 3H), 3.14 (t, J = 6.9 Hz, 2H), 3.06-2.96 |
| | | | | (m, 1H), 2.96-2.83 (m, 4H), 2.78-2.58 (m, 2H), 2.05- |
| | | | | 1.94 (m, 1H), 1.32-1.18 (m, 1H), 1.07-0.92 (m, 1H) |
| K241 | AM | P | 939.5 | 11.07 (s, 1H), 8.96-8.89 (m, 1H), 8.77 (d, J = 5.1 Hz, |
| | | | | 1H), 8.68-8.61 (m, 2H), 8.26-8.22 (m, 1H), 8.02 (dd, |
| | | | | J = 7.4, 1.8 Hz, 1H), 7.95-7.87 (m, 2H), 7.56 (dd, J = |
| | | | | 7.1, 1.7 Hz, 1H), 7.53-7.47 (m, 1H), 7.09-7.03 (m, |
| | | | | 1H), 7.02-6.96 (m, 1H), 6.89 (dd, J = 8.0, 1.6 Hz, |
| | | | | 1H), 6.44 (t, J = 7.2 Hz, 1H), 6.39 (s, 1H), 5.33 (dd, J = |
| | | | | 12.6, 5.3 Hz, 1H), 5.01-4.74 (m, 1H), 3.62-3.42 (m, |
| | | | | 16H), 3.37-3.5 (m, 2H), 3.31 (s, 3H), 3.05-2.96 (m, |
| | | | | 1H), 2.95-2.77 (m, 6H), 2.77-2.56 (m, 2H), 2.05-1.94 |
| | | | | (m, 1H), 1.31-1.19 (m, 1H), 1.06-0.92 (m, 1H) |
| K245 | AP | P | 1027.4 | 11.07 (s, 1H), 8.96-8.90 (m, 1H), 8.78 (d, J = 5.1 Hz, |
| | | | | 1H), 8.69-8.62 (m, 2H), 8.24 (s, 1H), 8.02 (dd, J = |
| | | | | 7.4, 1.7 Hz, 1H), 7.93 (s, 1H), 7.90 (dd, J = 5.2, 1.5 |
| | | | | Hz, 1H), 7.56 (dd, J = 7.1, 1.7 Hz, 1H), 7.53-7.46 (m, |
| | | | | 1H), 7.09-7.05 (m, 1H), 7.02-6.97 (m, 1H), 6.92-6.88 |
| | | | | (m, 1H), 6.47-6.41 (m, 1H), 6.39 (s, 1H), 5.33 (dd, J = |
| | | | | 12.6, 5.4 Hz, 1H), 5.95-4.75 (m, 1H), 3.63-3.43 (m, |
| | | | | 24H), 3.40-3.37 (m, 2H), 3.31 (s, 3H), 3.05-2.96 (m, |
| | | | | 1H), 2.95-2.77 (m, 6H), 2.75-2.56 (m, 2H), 2.06- |
| | | | | 1.94 (m, 1H), 1.31-1.19 (m, 1H), 1.05-0.93 (m, 1H) |
| K246 | EG | P | 1027.5 | 11.08 (s, 1H), 8.96-8.91 (m, 1H), 8.77 (d, J = 5.1 Hz, |
| | | | | 1H), 8.70-8.64 (m, 2H), 8.26-8.23 (m, 1H), 8.02 (dd, |
| | | | | J = 7.3, 1.8 Hz, 1H), 7.96-7.93 (m, 1H), 7.90 (dd, J = |
| | | | | 5.2, 1.5 Hz, 1H), 7.56 (dd, J = 7.0, 1.7 Hz, 1H), 7.54- |
| | | | | 7.48 (m, 1H), 7.00-6.94 (m, 2H), 6.94-6.88 (m, 1H), |
| | | | | 6.44 (t, J = 7.2 Hz, 1H), 6.40 (s, 1H), 5.36 (dd, J = |
| | | | | 12.5, 5.4 Hz, 1H), 4.99-4.78 (m, 1H), 3.66 (t, J = 6.9 |
| | | | | Hz, 2H), 3.61-3.43 (m, 22H), 3.41-3.39 (m, 2H), 3.38 |
| | | | | (s, 3H), 3.14 (t, J = 6.9 Hz, 2H), 3.04-2.97 (m, 1H), |
| | | | | 2.95-2.82 (m, 4H), 2.75-2.56 (m, 2H), 2.06-1.91 (m, |
| | | | | 1H), 1.37-1.11 (m, 1H), 1.06-0.93 (m, 1H) |
| K252 | AQ | EM | 697.2 | 11.09 (s, 1H), 10.41 (s, 1H), 8.72-8.69 (m, 1H), 7.94 |
| | | | | (d, J = 8.2 Hz, 1H), 7.89 (s, 1H), 7.70 (d, J = 7.8 Hz, |
| | | | | 1H), 7.66-7.61 (m, 1H), 7.60-7.55 (m, 1H), 7.47 (t, J = |
| | | | | 7.7 Hz, 1H), 7.08 (t, J = 7.6 Hz, 1H), 7.03 (s, 1H), |
| | | | | 6.97 (d, J = 8.0 Hz, 1H), 6.87-6.83 (m, 1H), 5.84 (s, |
| | | | | 1H), 5.32 (dd, J = 12.9, 5.3 Hz, 1H), 4.96-4.61 (m, |
| | | | | 1H), 3.33 (s, 3H), 3.03-3.00 (m, 2H), 2.89 (d, J = 4.9 |
| | | | | Hz, 3H), 2.75-2.66 (m, 2H), 2.66-2.58 (m, 4H), 2.05- |
| | | | | 1.96 (m, 1H), 1.74-1.72 (m, 2H), 1.64-1.61 (m, 1H), |
| | | | | 1.56-1.52 (m, 1H), 1.22-1.10 (m, 1H), 0.84-0.70 (m, |
| | | | | 1H) |
| K255 | BT | EM | 725.4 | 11.09 (s, 1H), 10.35 (s, 1H), 8.71 (d, J = 4.1 Hz, 1H), |
| | | | | 8.64 (s, 1H), 7.95-7.91 (m, 1H), 7.88 (s, 1H), 7.70 (d, |
| | | | | J = 7.3 Hz, 1H), 7.59-7.54 (m, 1H), 7.50-7.44 (m, |
| | | | | 1H), 7.12-7.07 (m, 1H), 7.01-6.96 (m, 2H), 6.85-6.81 |
| | | | | (m, 1H), 5.84 (s, 1H), 5.33 (dd, J = 12.8, 5.2 Hz, 1H), |
| | | | | 4.90-4.68 (m, 1H), 3.31 (s, 3H), 3.27-3.23 (m, 2H), |
| | | | | 2.95-2.91 (m, 1H), 2.89 (d, J = 4.9 Hz, 3H), 2.87- |
| | | | | 2.83 (m, 1H), 2.76-2.69 (m, 1H), 2.69-2.63 (m, 1H), |
| | | | | 2.62-2.56 (m, 2H), 2.05-1.97 (m, 1H), 1.61-1.46 (m, |
| | | | | 4H), 1.36-1.27 (m, 4H), 1.21-1.11 (m, 1H), 0.85-0.72 |
| | | | | (m, 1H) |
| K279 | CR | I | 846.5 | 11.12 (s, 1H), 8.69-8.65 (m, 3H), 8.07 (dd, J = 8.3, |
| | | | | 2.3 Hz, 1H), 8.02-7.92 (m, 3H), 7.70-7.56 (m, 1H), |
| | | | | 7.52 (d, J = 5.2 Hz, 1H), 7.08 (s, 1H), 7.01-6.76 (m, |
| | | | | 2H), 6.47-6.39 (m, 2H), 5.39 (dd, J = 12.6, 5.5 Hz, |
| | | | | 1H), 4.99-4.79 (m, 1H), 4.40-4.36 (m, 1H), 3.75-3.53 |
| | | | | (m, 6H), 3.02-2.81 (m, 8H), 2.79-2.57 (m, 2H), 2.21- |
| | | | | 2.09 (m, 1H), 2.07-1.95 (m, 1H), 1.91-1.58 (m, 6H), |
| | | | | 1.28-1.19 (m, 1H), 1.06-0.96 (m, 1H) |
| K280 | CB | I | 846.5 | 11.07 (s, 1H), 8.78-8.47 (m, 3H), 8.10 (dd, J = 25.8, |
| | | | | 8.2 Hz, 1H), 8.03-7.88 (m, 3H), 7.60 (d, J = 7.1 Hz, |
| | | | | 1H), 7.52 (d, J = 6.0 Hz, 1H), 7.00-6.72 (m, 2H), |
| | | | | 6.67-6.57 (m, 1H), 6.46-6.38 (m, 2H), 5.33-5.26 (m, |
| | | | | 1H), 4.99-4.76 (m, 1H), 3.65-3.61 (m, 1H), 3.55-3.45 |
| | | | | (m, 1H), 3.32 (s, 3H), 3.29-3.25 (m, 2H), 3.09-2.97 |
| | | | | (m, 4H), 290-2.85 (m, 4H), 2.77-2.55 (m, 4H), 2.11- |
| | | | | 1.55 (m, 3H), 1.48-1.17 (m, 3H), 1.13-0.80 (m, 2H) |
| K253 | N | EM | 697.4 | 11.01 (s, 1H), 10.36 (s, 1H), 8.72-8.66 (m, 2H), 7.94 |
| | | | | (d, J = 8.3 Hz, 1H), 7.89 (s, 1H), 7.73-7.67 (m, 1H), |
| | | | | 7.59-7.53 (m, 1H), 7.47 (t, J = 8.1 Hz, 1H), 7.09 (t, J = |
| | | | | 7.5 Hz, 1H), 6.96-6.90 (m, 2H), 6.89-6.83 (m, 1H), |
| | | | | 5.85 (s, 1H), 5.36 (dd, J = 12.6, 5.4 Hz, 1H), 4.90- |
| | | | | 4.68 (m, 1H), 3.55 (s, 3H), 2.94-2.91 (m, 2H), 2.90 |
| | | | | (d, J = 5.0 Hz, 3H), 2.88-2.84 (m, 1H), 2.76-2.57 (m, |
| | | | | 3H), 2.04-1.95 (m, 1H), 1.64-1.62 (m, 2H), 1.27-1.19 |
| | | | | (m, 2H), 1.19-1.09 (m, 2H), 0.89-0.72 (m, 2H) |
| K256 | CL | EM | 753.4 | 11.16-10.99 (m, 1H), 10.33 (s, 1H), 8.71 (d, J = 4.1 |
| | | | | Hz, 1H), 8.62 (t, J = 5.7 Hz, 1H), 7.92 (d, J = 8.3 Hz, |
| | | | | 1H), 7.88 (s, 1H), 7.70 (dd, J = 7.9, 1.5 Hz, 1H), |
| | | | | 7.57-7.53 (m, 1H), 7.50-7.44 (m, 1H), 7.09 (t, J = 7.5 |
| | | | | Hz, 1H), 7.00-6.96 (m, 2H), 6.86-6.81 (m, 1H), 5.83 |
| | | | | (s, 1H), 5.32 (dd, J = 12.9, 5.3 Hz, 1H), 4.89-4.69 |
| | | | | (m, 1H), 3.31 (s, 3H), 3.27-3.21 (m, 2H), 2.96-2.92 |
| | | | | (m, 1H), 2.90 (d, J = 4.9 Hz, 3H), 2.88-2.84 (m, 1H), |
| | | | | 2.75-2.69 (m, 1H), 2.69-2.62 (m, 1H), 2.61-2.54 (m, |
| | | | | 2H), 2.04-1.96 (m, 1H), 1.58-1.45 (m, 4H), 1.26 (s, |
| | | | | 8H), 1.19-1.09 (m, 1H), 0.83-0.72 (m, 1H) |
| K281 | CC | I | 860.5 | 11.14-11.08 (m, 1H), 8.71-8.58 (m, 3H), 8.19-8.06 |
| | | | | (m, 1H), 8.04-7.97 (m, 2H), 7.96-7.90 (m, 2H), 7.63- |
| | | | | 7.56 (m, 1H), 7.52 (d, J = 5.4 Hz, 1H), 7.07 (t, J = |
| | | | | 9.2 Hz, 1H), 7.00-6.80 (m, 1H), 6.47-6.36 (m, 2H), |
| | | | | 5.44-5.31 (m, 1H), 4.99-4.76 (m 1H), 3.78-3.52 (m, |
| | | | | 5H), 3.45-3.38 (m, 3H), 3.22-3.10 (m, 1H), 3.05-2.93 |
| | | | | (m, 4H), 2.87 (d, J = 4.9 Hz, 3H), 2.80-2.56 (m, 2H), |
| | | | | 2.52-2.51 (m, 2H), 2.49-2.48 (m, 1H), 2.09-1.87 (m, |
| | | | | 3H), 1.85-1.42 (m, 3H), 1.33-1.12 (m, 1H), 1.07-0.80 |
| | | | | (m, 1H) |
| K112 | DP | M | 862.4 | 11.07 (s, 1H), 10.71 (s, 1H), 10.08 (s, 1H), 9.05-9.01 |
| | | | | (m, 2H), 8.69-8.65 (m, 1H), 8.59-8.50 (m, 2H), 8.48- |
| | | | | 8.42 (m, 1H), 8.10-8.02 (m, 1H), 7.97 (s, 1H), 7.73 |
| | | | | (d, J = 7.9 Hz, 1H), 7.64 (d, J = 8.8 Hz, 1H), 7.43 |
| | | | | (dd, J = 7.7, 1.6 Hz, 1H), 7.37-7.30 (m, 1H), 7.07- |
| | | | | 7.03 (m, 1H), 7.02-6.96 (m, 1H), 6.92-6.86 (m, 1H), |
| | | | | 5.33 (dd, J = 12.7, 5.3 Hz, 1H), 3.70 (s, 3H), 3.61 (t, |
| | | | | J = 7.1 Hz, 2H), 3.58-3.51 (m, 6H), 3.45-3.38 (m, |
| | | | | 2H), 3.31 (s, 3H), 2.96-2.84 (m, 1H), 2.84-2.76 (m, |
| | | | | 5H), 2.76-2.56 (m, 2H), 2.05-1.92 (m, 1H) |
| K113 | AM | M | 950.4 | 11.07 (s, 1H), 10.73 (s, 1H), 10.15 (s, 1H), 9.07-9.01 |
| | | | | (m, 2H), 8.71-8.65 (m, 1H), 8.63-8.55 (m, 1H), 8.54- |
| | | | | 8.44 (m, 2H), 8.14 (s, 1H), 8.12-8.05 (m, 1H), 7.75- |
| | | | | 7.69 (m, 1H), 7.63-7.56 (m, 1H), 7.4-7.42 (m, 1H), |
| | | | | 7.38-7.31 (m, 1H), 7.06-7.04 (m, 1H), 7.02-6.97 (m, |
| | | | | 1H), 6.92-6.86 (m, 1H), 5.33 (dd, J = 12.6, 5.2 Hz, |
| | | | | 1H), 3.70 (s, 3H), 3.62-3.56 (m, 2H), 3.56-3.51 (m, |
| | | | | 6H), 3.51-3.45 (m, 8H), 3.45-3.49 (m, 2H), 3.32 (s, |
| | | | | 3H), 2.96-2.84 (m, 1H), 2.84-2.77 (m, 5H), 2.76-2.58 |
| | | | | (m, 2H), 2.04-1.94 (m, 1H) |
| K114 | N | M | 802.4 | 11.07 (s, 1H), 10.71 (s, 1H), 10.07 (s, 1H), 9.04 (s, |
| | | | | 2H), 8.65 (d, J = 2.4 Hz, 1H), 8.54 (d, J = 8.7 Hz, |
| | | | | 2H), 8.42-8.38 (m, 1H), 8.22 (s, 1H), 8.11-8.02 (m, |
| | | | | 1H), 7.96 (s, 1H), 7.76-7.71 (m, 1H), 7.65 (d, J = 8.8 |
| | | | | Hz, 1H), 7.46-7.40 (m, 1H), 7.38-7.27 (m, 1H), 6.96 |
| | | | | (d, J = 4.7 Hz, 2H), 6.92-6.87 (m, 1H), 5.36 (dd, J = |
| | | | | 12.5, 5.4 Hz, 1H), 3.70 (s, 3H), 3.56 (s, 3H), 2.97- |
| | | | | 2.84 (m, 3H), 2.80 (d, J = 4.4 Hz, 3H), 2.69-2.58 (m, |
| | | | | 3H), 2.02-1.97 (m, 1H), 1.69-1.63 (m, 4H) |
| K124 | EG | M | 1038.5 | 11.09 (s, 1H), 10.72 (s, 1H), 10.09 (s, 1H), 9.08-8.99 |
| | | | | (m, 2H), 8.71-8.65 (m, 1H), 8.59-8.51 (m, 2H), 8.49- |
| | | | | 8.43 (m, 1H), 8.10-8.04 (m, 1H), 7.99 (s, 1H), 7.73 |
| | | | | (dd, J = 8.0, 1.7 Hz, 1H), 7.64 (d, J = 8.8 Hz, 1H). |
| | | | | 7.43 (dd, J = 7.8, 1.7 Hz, 1H), 7.34 (t, J = 7.9 Hz, |
| | | | | 1H), 7.02-6.82 (m, 3H), 5.36 (dd, J = 12.6, 5.3 Hz, |
| | | | | 1H), 3.70 (s, 3H), 3.66 (t, J = 6.9 Hz, 2H), 3.57 (s, |
| | | | | 3H), 3.55-3.48 (m, 6H), 3.48-3.44 (m, 16H), 3.44- |
| | | | | 3.38 (m, 2H), 3.14 (t, J = 6.9 Hz, 2H), 2.97-2.82 (m, |
| | | | | 1H), 2.80 (d, J = 4.3 Hz, 3H), 2.75-2.58 (m, 2H), |
| | | | | 2.04-1.94 (m, 1H) |
| K107 | AQ | M | 802.3 | 11.07 (s, 1H), 10.72 (s, 1H), 10.08 (s, 1H), 9.07-9.00 |
| | | | | (m, 2H), 8.71-8.62 (m, 1H), 8.61-8.47 (m, 3H), 8.46- |
| | | | | 8.35 (m, 1H), 8.11-8.02 (m, 1H), 7.75-7.67 (m, 1H), |
| | | | | 7.66-7.57 (m, 1H), 7.50-7.39 (m, 1H), 7.39-7.28 (m, |
| | | | | 1H), 7.06-7.03 (m, 1H), 7.03-6.97 (m, 1H), 6.91-6.84 |
| | | | | (dd, J = 8.0, 1.6 Hz, 1H), 5.33 (dd, J = 12.7, 5.4 Hz, |
| | | | | 1H), 3.70 (s, 3H), 3.34(s, 3H), 3.30-3.26 (m, 2H), |
| | | | | 2.97-2.85 (m, 1H), 2.81 (d, J = 4.4 Hz, 3H), 2.75- |
| | | | | 2.58 (m, 4H), 2.07-1.91 (m, 1H), 1.70-1.44 (m, 4H) |
| K110 | BT | M | 830.4 | 11.06 (s, 1H), 10.71 (s, 1H), 10.06 (s, 1H), 9.04 (s, |
| | | | | 2H), 8.65 (d, J = 2.4 Hz, 1H), 8.57-8.50 (m, 2H), |
| | | | | 8.36 (t, J = 5.6 Hz, 1H), 8.30-8.22 (m, 1H), 8.05 (dd, |
| | | | | J = 8.8, 2.4 Hz, 1H), 7.96 (s, 1H), 7.73 (dd, J = 8.0, |
| | | | | 1.7 Hz, 1H), 7.64 (d, J = 8.8 Hz, 1H), 7.43 (dd, J = |
| | | | | 7.8, 1.7 Hz, 1H), 7.33 (t, J = 7.9 Hz, 1H), 7.03-6.96 |
| | | | | (m, 2H), 6.86 (dd, J = 8.2, 1.6 Hz, 1H), 5.33 (dd, J = |
| | | | | 12.7, 5.4 Hz, 1H), 3.70 (s, 3H), 3.32 (s, 3H), 3.26- |
| | | | | 3.23 (m, 2H), 2.91-2.86 (m, 1H), 2.80 (d, J = 4.4 Hz, |
| | | | | 3H), 2.77-2.65 (m, 2H), 2.64-2.59 (m, 2H), 2.04- |
| | | | | 1.96 (m, 1H), 1.65-1.57 (m, 2H), 1.55-1.47 (m, 2H), |
| | | | | 1.40-1.29 (m, 4H) |
| K111 | CL | M | 858.4 | 11.07 (s, 1H), 10.71 (s, 1H), 10.06 (s, 1H), 9.06-9.02 |
| | | | | (m, 2H), 8.66-8.64 (m, 1H), 8.57-8.52 (m, 2H), 8.38- |
| | | | | 8.33 (m, 1H), 8.05 (dd, J = 8.8, 2.4 Hz, 1H), 7.96 (s, |
| | | | | 1H), 7.73 (dd, J = 8.0, 1.7 Hz, 1H), 7.64 (d, J = 8.8 |
| | | | | Hz, 1H), 7.43 (dd, J = 7.8, 1.6 Hz, 1H), 7.34 (t, J = |
| | | | | 7.9 Hz, 1H), 7.03-6.96 (m, 2H), 6.88-6.82 (m, 1H), |
| | | | | 5.33 (dd, J = 12.7, 5.4 Hz, 1H), 3.70 (s, 3H), 3.32 (s, |
| | | | | 3H), 3.26-3.20 (m, 2H), 2.95-2.84 (m, 1H), 2.80 (d, J = |
| | | | | 4.4 Hz, 3H), 2.76-2.56 (m, 4H), 2.05-1.95 (m, 1H), |
| | | | | 1.64-1.46 (m, 4H), 1.37-1.23 (m, 8H) |
| K115 | BV | M | 830.4 | 11.08 (s, 1H), 10.72 (s, 1H), 10.08 (s, 1H), 9.06-9.02 |
| | | | | (m, 2H), 8.69-8.63 (m, 1H), 8.52 (s, 1H), 8.41-8.35 |
| | | | | (m, 1H), 8.14 (s, 1H), 8.11-8.03 (m, 1H), 8.00-7.91 |
| | | | | (m, 1H), 7.75-7.70 (m, 1H), 7.66-7.60 (m, 1H), 7.48- |
| | | | | 7.39 (m, 1H), 7.38-7.30 (m, 1H), 7.00-6.91 (m, 2H), |
| | | | | 6.89-6.83 (m, 1H), 5.36 (dd, J = 12.4, 5.4 Hz, 1H), |
| | | | | 3.70 (s, 3H), 3.55 (s, 3H), 3.31-3.20 (m, 2H), 2.95- |
| | | | | 2.83 (m, 3H), 2.81 (d, J = 4.4 Hz, 3H), 2.76-2.58 (m, |
| | | | | 2H), 2.07-1.91 (m, 1H), 1.68-1.48 (m, 4H), 1.46-1.33 |
| | | | | (m, 4H) |
| K116 | CI | M | 858.4 | 11.08 (s, 1H), 10.75 (s, 1H), 10.19-9.95 (m, 1H), |
| | | | | 9.07-9.02 (m, 2H), 8.74-8.65 (m, 1H), 8.51 (s, 1H), |
| | | | | 8.46-8.31 (m, 1H), 8.20-8.02 (m, 2H), 8.01-7.90 (m, |
| | | | | 1H), 7.76-7.65 (m, 1H), 7.58-7.44 (m, 2H), 7.41-7.32 |
| | | | | (m, 1H), 7.00-6.90 (m, 2H), 6.88-6.81 (m, 1H), 5.36 |
| | | | | (dd, J = 12.6, 5.4 Hz, 1H), 3.70 (s, 3H), 3.55 (s, 3H), |
| | | | | 3.28-3.18 (m, 2H), 2.94-2.83 (m, 3H), 2.82 (d, J = |
| | | | | 4.3 Hz, 3H), 2.77-2.59 (m, 2H), 2.02-1.94 (m, 1H), |
| | | | | 1.56 (d, J = 28.8 Hz, 4H), 1.44-1.22 (m, 8H) |
| K122 | AP | M | 1038.5 | 11.08 (s, 1H), 10.72 (s, 1H), 10.09 (s, 1H), 9.09-9.01 |
| | | | | (m, 2H), 8.70-8.64 (m, 1H), 8.60-8.51 (m, 2H), 8.50- |
| | | | | 8.43 (m, 1H), 8.07 (dd, J = 8.8, 2.4 Hz, 1H), 7.99 (s, |
| | | | | 1H), 7.73 (dd, J = 7.9, 1.7 Hz, 1H), 7.64 (d, J = 8.8 |
| | | | | Hz, 1H), 7.43 (dd, J = 7.7, 1.6 Hz, 1H), 7.34 (t, J = |
| | | | | 7.9 Hz, 1H), 7.08-7.04 (m, 1H), 7.00 (d, J = 8.0 Hz, |
| | | | | 1H), 6.90 (dd, J = 8.1, 1.5 Hz, 1H), 5.33 (dd, J = |
| | | | | 12.7, 5.3 Hz, 1H), 3.70 (s, 3H), 3.60 (t, J = 7.0 Hz, |
| | | | | 2H), 3.56-3.51 (m, 6H), 3.51-3.45 (m, 16H), 3.44- |
| | | | | 3.37 (m, 2H), 3.31 (s, 3H), 2.95-2.85 (m, 1H), 2.85- |
| | | | | 2.77 (m, 5H), 2.76-2.58 (m, 2H), 2.06-1.93 (m, 1H) |
| K123 | EI | M | 862.4 | 11.08 (s, 1H), 10.71 (s, 1H), 10.09 (s, 1H), 9.06-9.02 |
| | | | | (m, 2H), 8.69-8.65 (m, 1H), 8.58-8.50 (m, 2H), 8.48- |
| | | | | 8.43 (m, 1H), 8.07 (dd, J = 8.8, 2.4 Hz, 1H), 7.98 (s, |
| | | | | 1H), 7.73 (dd, J = 8.0, 1.7 Hz, 1H), 7.64 (d, J = 8.8 |
| | | | | Hz, 1H), 7.43 (dd, J = 7.8, 1.6 Hz, 1H), 7.34 (t, J = |
| | | | | 7.9 Hz, 1H), 6.99-6.93 (m, 2H), 6.93-6.87 (m, 1H), |
| | | | | 5.36 (dd, J = 12.6, 5.3 Hz, 1H), 3.70 (s, 3H), 3.67 (t, |
| | | | | J = 7.0 Hz, 2H), 3.58-3.49 (m, 6H), 3.44-3.36 (m, |
| | | | | 2H), 3.31 (s, 3H), 3.13 (t, J = 6.9 Hz, 2H), 2.99-2.84 |
| | | | | (m, 1H), 2.80 (d, J = 4.4 Hz, 3H), 2.76-2.55 (m, 2H), |
| | | | | 2.06-1.88 (m, 1H) |
| K125 | EJ | M | 950.4 | 11.08 (s, 1H), 10.72 (s, 1H), 10.09 (s, 1H), 9.05-9.03 |
| | | | | (m, 2H), 8.69-8.65 (m, 1H), 8.59-8.51 (m, 2H), 8.49- |
| | | | | 8.43 (m, 1H), 8.07 (dd, J = 8.8, 2.4 Hz, 1H), 7.98 (s, |
| | | | | 1H), 7.73 (dd, J = 8.0, 1.7 Hz, 1H), 7.63 (d, J = 8.8 |
| | | | | Hz, 1H), 7.43 (dd, J = 7.8, 1.6 Hz, 1H), 7.34 (t, J = |
| | | | | 7.9 Hz, 1H), 7.01-6.92 (m, 2H), 6.92-6.87 (m, 1H), |
| | | | | 5.36 (dd, J = 12.5, 5.4 Hz, 1H), 3.70 (s, 3H), 3.65 (t, |
| | | | | J = 6.9 Hz, 2H), 3.56 (s, 3H), 3.55-3.43 (m, 14H), |
| | | | | 3.43-3.37 (m, 2H), 3.13 (t, J = 6.9 Hz, 2H), 2.97-2.84 |
| | | | | (m, 1H), 2.80 (d, J = 4.4 Hz, 3H), 2.75-2.57 (m, 2H), |
| | | | | 2.04-1.93 (m, 1H) |
| K321 | AQ | CZ | 787.4 | 11.08 (s, 1H), 9.02 (d, J = 2.4 Hz, 1H), 8.79 (t, J = |
| | | | | 5.6 Hz, 1H), 8.73 (d, J = 8.0 Hz, 1H), 8.70 (s, 1H), |
| | | | | 8.39 (dd, J = 8.5, 2.4 Hz, 1H), 8.12 (dd, J = 7.3, 1.8 |
| | | | | Hz, 1H), 7.99 (d, J = 8.4 Hz, 1H), 7.86 (s, 1H), 7.61 |
| | | | | (dd, J = 7.1, 1.7 Hz, 1H), 7.53-7.48 (m, 1H), 7.06 (d, |
| | | | | J = 1.5 Hz, 1H), 7.01 (d, J = 8.0 Hz, 1H), 6.90 (dd, J = |
| | | | | 8.0, 1.6 Hz, 1H), 6.48 (t, J = 7.2 Hz, 1H), 6.40 (s, |
| | | | | 1H), 5.34 (dd, J = 12.8, 5.4 Hz, 1H), 4.57-4.47 (m, |
| | | | | 1H), 3.39-3.35 (m, 5H), 2.97-2.80 (m, 4H), 2.77-2.56 |
| | | | | (m, 4H), 2.36-2.28 (m, 2H), 2.05-1.89 (m, 3H), 1.80- |
| | | | | 1.64 (m, 4H), 1.63-1.55 (m, 2H) |
| K322 | AQ | DA | 773.3 | 11.07 (s, 1H), 9.02 (d, J = 2.3 Hz, 1H), 8.79 (t, J = |
| | | | | 5.6 Hz, 1H), 8.68 (s, 1H), 8.60 (d, J = 4.0 Hz, 1H), |
| | | | | 8.39 (dd, J = 8.4, 2.3 Hz, 1H), 8.03 (dd, J = 7.4, 1.8 |
| | | | | Hz, 1H), 7.98 (d, J = 8.5 Hz, 1H), 7.87 (s, 1H), 7.61 |
| | | | | (dd, J = 7.1, 1.7 Hz, 1H), 7.52-7.46 (m, 1H), 7.06 (d, |
| | | | | J = 1.5 Hz, 1H), 7.01 (d, J = 8.0 Hz, 1H), 6.94-6.85 |
| | | | | (m, 1H), 6.48 (t, J = 7.2 Hz, 1H), 6.38 (s, 1H), 5.34 |
| | | | | (dd, J = 12.7, 5.3 Hz, 1H), 3.38-3.34 (m, 4H), 2.96- |
| | | | | 2.89 (m, 2H), 2.87 (d, J = 4.9 Hz, 4H), 2.78-2.56 (m, |
| | | | | 4H), 2.07-1.95 (m, 1H), 1.80-1.53 (m, 4H), 0.89-0.72 |
| | | | | (m, 2H), 0.67-0.51 (m, 2H) |
| K126 | AQ | BU | 802.4 | 11.07 (s, 1H), 10.71 (s, 1H), 9.90 (s, 1H), 9.06-9.03 |
| | | | | (m, 2H), 8.61 (t, J = 5.7 Hz, 1H), 8.56-8.49 (m, 2H), |
| | | | | 8.27 (d, J = 5.2 Hz, 1H), 7.97 (s, 1H), 7.89 (s, 1H), |
| | | | | 7.72 (dd, J = 8.0, 1.7 Hz, 1H), 7.42 (dd, J = 7.8, 1.6 |
| | | | | Hz, 1H), 7.33 (t, J = 7.9 Hz, 1H), 7.18 (dd, J = 5.3, |
| | | | | 1.5 Hz, 1H), 7.06-7.04 (m, 1H), 7.03-6.98 (m, 1H), |
| | | | | 6.91-6.87 (m, 1H), 5.34 (dd, J = 12.7, 5.4 Hz, 1H), |
| | | | | 3.70 (s, 3H), 3.38(s, 3H), 3.29-3.24(m, 2H), 2.96- |
| | | | | 2.84 (m, 1H), 2.80 (d, J = 4.4 Hz, 3H), 2.76-2.58 (m, |
| | | | | 4H), 2.05-1.95 (m, 1H), 1.70-1.49 (m, 4H) |
| K127 | BT | BU | 830.4 | 11.07 (s, 1H), 10.71 (s, 1H), 9.90 (s, 1H), 9.06-9.04 |
| | | | | (m, 2H), 8.58 (t, J = 5.6 Hz, 1H), 8.55-8.50 (m, 2H), |
| | | | | 8.27 (d, J = 5.2 Hz, 1H), 7.97 (s, 1H), 7.88 (s, 1H), |
| | | | | 7.72 (dd, J = 8.0, 1.7 Hz, 1H), 7.42 (dd, J = 7.8, 1.6 |
| | | | | Hz, 1H), 7.33 (t, J = 7.8 Hz, 1H), 7.19 (dd, J = 5.3, |
| | | | | 1.5 Hz, 1H), 7.04-6.96 (m, 2H), 6.89-6.84 (m, 1H), |
| | | | | 5.33 (dd, J = 12.7, 5.4 Hz, 1H), 3.70 (s, 3H), 3.38 (s, |
| | | | | 3H), 3.27-3.20 (m, 2H), 2.98-2.84 (m, 1H), 2.80 (d, J = |
| | | | | 4.4 Hz, 3H), 2.74-2.57 (m, 4H), 2.06-1.93 (m, 1H), |
| | | | | 1.66-1.45 (m, 4H), 1.44-1.29 (m, 4H) |
| K129 | CL | BU | 858.4 | 11.07 (s, 1H), 10.71 (s, 1H), 9.90 (s, 1H), 9.05-9.03 |
| | | | | (m, 2H), 8.58 (t, J = 5.6 Hz, 1H), 8.56-8.48 (m, 2H), |
| | | | | 8.27 (d, J = 5.2 Hz, 1H), 7.97 (s, 1H), 7.88 (s, 1H), |
| | | | | 7.72 (dd, J = 7.9, 1.7 Hz, 1H), 7.42 (dd, J = 7.8, 1.6 |
| | | | | Hz, 1H), 7.33 (t, J = 7.8 Hz, 1H), 7.18 (dd, J = 5.2, |
| | | | | 1.5 Hz, 1H), 7.03-6.97 (m, 2H), 6.88-6.83 (m, 1H), |
| | | | | 5.33 (dd, J = 12.6, 5.4 Hz, 1H), 3.70 (s, 3H), 3.37 (s, |
| | | | | 3H), 3.26-3.18 (m, 2H), 2.96-2.83 (m, 1H), 2.80 (d, J = |
| | | | | 4.4 Hz, 3H), 2.75-2.57 (m, 4H), 2.07-1.94 (m, 1H), |
| | | | | 1.63-1.46 (m, 4H), 1.31 (s, 8H) |
| K128 | N | BU | 802.4 | 11.08 (s, 1H), 10.71 (s, 1H), 9.90 (s, 1H), 9.05-9.04 |
| | | | | (m, 2H), 8.62 (t, J = 5.7 Hz, 1H), 8.55-8.49 (m, 2H), |
| | | | | 8.27 (d, J = 5.2 Hz, 1H), 7.98 (s, 1H), 7.88 (s, 1H), |
| | | | | 7.73 (dd, J = 8.0, 1.7 Hz, 1H), 7.42 (dd, J = 7.8, 1.7 |
| | | | | Hz, 1H), 7.33 (t, J = 7.9 Hz, 1H), 7.18 (dd, J = 5.3, |
| | | | | 1.5 Hz, 1H), 6.99-6.96 (m, 2H), 6.93-6.87 (m, 1H), |
| | | | | 5.37 (dd, J = 12.5, 5.4 Hz, 1H), 3.71 (s, 3H), 3.56 (s, |
| | | | | 3H), 3.30-3.25(m, 2H), 2.98-2.91 (m, 2H), 2.90-2.84 |
| | | | | (m, 1H), 2.80 (d, J = 4.4 Hz, 3H), 2.76-2.58 (m, 2H), |
| | | | | 2.05-1.96 (m, 1H), 1.70-1.61 (m, 4H) |
| K193 | BV | BU | 830.4 | 11.08 (s, 1H), 10.71 (s, 1H), 9.90 (s, 1H), 9.06-9.02 |
| | | | | (m, 2H), 8.59 (t, J = 5.6 Hz, 1H), 8.55-8.50 (m, 2H), |
| | | | | 8.27 (d, J = 5.2 Hz, 1H), 7.97 (s, 1H), 7.89 (s, 1H), |
| | | | | 7.73 (dd, J = 8.0, 1.7 Hz, 1H), 7.42 (dd, J = 7.8, 1.6 |
| | | | | Hz, 1H), 7.33 (t, J = 7.9 Hz, 1H), 7.19 (dd, J = 5.2, |
| | | | | 1.5 Hz, 1H), 7.00-6.92 (m, 2H), 6.90-6.83 (m, 1H), |
| | | | | 5.36 (dd, J = 12.5, 5.4 Hz, 1H), 3.70 (s, 3H), 3.56 (s, |
| | | | | 3H), 3.30-3.22 (m, 2H), 2.98-2.83 (m, 3H), 2.80 (d, J = |
| | | | | 4.4 Hz, 3H), 2.76-2.58 (m, 2H), 2.05-1.93 (m, 1H), |
| | | | | 1.68-1.49 (m, 4H), 1.48-1.33 (m, 4H) |
| K130 | CI | BU | 858.4 | 11.08 (s, 1H), 10.71 (s, 1H), 9.90 (s, 1H), 9.06-9.02 |
| | | | | (m, 2H), 8.58 (t, J = 5.6 Hz, 1H), 8.55-8.49 (m, 2H), |
| | | | | 8.27 (d, J = 5.2 Hz, 1H), 7.97 (s, 1H), 7.88 (s, 1H), |
| | | | | 7.72 (dd, J = 8.0, 1.7 Hz, 1H), 7.42 (dd, J = 7.9, 1.7 |
| | | | | Hz, 1H), 7.33 (t, J = 7.8 Hz, 1H), 7.19 (dd, J = 5.3, |
| | | | | 1.5 Hz, 1H), 6.98-6.90 (m, 2H), 6.89-6.83 (m, 1H), |
| | | | | 5.36 (dd, J = 12.5, 5.5 Hz, 1H), 3.70 (s, 3H), 3.55 (s, |
| | | | | 3H), 3.28-3.20 (m, 2H), 2.95-2.83 (m, 3H), 2.80 (d, J = |
| | | | | 4.4 Hz, 3H), 2.76-2.57 (m, 2H), 2.05-1.93 (m, 1H), |
| | | | | 1.66-1.47 (m, 4H), 1.44-1.19 (m, 8H) |
| K192 | N | DQ | 676.4 | 11.08 (s, 1H), 10.53 (s, 1H), 10.01 (s, 1H), 8.61 (d, J = |
| | | | | 2.4 Hz, 1H), 8.54-8.48 (m, 2H), 8.40 (t, J = 5.6 Hz, |
| | | | | 1H), 8.04 (dd, J = 8.8, 2.4 Hz, 1H), 7.88 (s, 1H), 7.61 |
| | | | | (d, J = 8.8 Hz, 1H), 7.43 (t, J = 7.8 Hz, 2H), 7.36- |
| | | | | 7.29 (m, 2H), 7.15 (t, J = 7.3 Hz, 1H), 6.99-6.94 (m, |
| | | | | 2H), 6.92-6.87 (m, 1H), 5.36 (dd, J = 12.5, 5.4 Hz, |
| | | | | 1H), 3.56 (s, 3H), 2.98-2.83 (m, 3H), 2.79 (d, J = 4.4 |
| | | | | Hz, 3H), 2.75-2.62 (m, 2H), 2.60-2.54 (m, 2H), 2.04- |
| | | | | 1.95 (m, 1H), 1.71-1.60 (m, 4H) |
| K191 | N | R | 706.4 | 11.08 (s, 1H), 10.58 (s, 1H), 8.84-8.80 (m, 1H), 8.75- |
| | | | | 8.72 (m, 1H), 8.59-8.55 (m, 1H), 8.46 (s, 1H), 8.21- |
| | | | | 8.17 (m, 1H), 7.47 (d, J = 7.7 Hz, 1H), 7.31-7.27 (m, |
| | | | | 2H), 7.21 (d, J = 8.1 Hz, 1H), 7.13-7.05 (m, 1H), |
| | | | | 6.99-6.95 (m, 2H), 6.93-6.86 (m, 3H), 5.41-5.32 (m, |
| | | | | 1H), 3.86 (s, 3H), 3.56 (s, 3H), 2.99-2.86 (m, 3H), |
| | | | | 2.82 (d, J = 4.4 Hz, 3H), 2.75-2.61 (m, 2H), 2.54- |
| | | | | 2.50 (m, 2H), 2.05-2.00 (m, 1H), 1.71-1.64 (m, 4H) |
| K190 | N | Q | 754.4 | 11.11-11.07 (m, 2H), 10.98 (s, 1H), 8.92-8.84 (m, |
| | | | | 1H), 8.73 (d, J = 2.3 Hz, 1H), 8.60 (t, J = 5.6 Hz, |
| | | | | 1H), 8.53 (s, 1H), 8.23-8.16 (m, 1H), 8.04 (d, J = 8.0 |
| | | | | Hz, 1H), 7.91-7.83 (m, 1H), 7.76 (d, J = 8.0 Hz, 1H), |
| | | | | 7.63-7.55 (m, 1H), 7.19 (d, J = 8.7 Hz, 1H), 6.97 (d, |
| | | | | J = 4.7 Hz, 2H), 6.92-6.85 (m, 1H), 6.82 (s, 1H), 5.34 |
| | | | | (dd, J = 12.7, 5.4 Hz, 1H), 3.22 (s, 3H), 3.07-2.86 |
| | | | | (m, 3H), 2.90-2.87 (m, 1H), 2.82 (d, J = 4.5 Hz, 3H), |
| | | | | 2.76-2.55 (m, 3H), 2.60 (t, J = 3.3 Hz, 1H), 2.33 (d, J = |
| | | | | 3.0 Hz, 1H), 2.12-2.00 (m, 1H), 2.00-1.88 (m, 1H), |
| | | | | 1.69-1.61 (m, 4H) |
| K189 | N | DR | 707.4 | 11.08 (s, 1H), 10.75 (s, 1H), 8.80-8.73 (m, 2H), 8.71- |
| | | | | 8.62 (m, 1H), 8.56-8.50 (m, 2H), 8.47 (s, 1H), 8.22- |
| | | | | 8.13 (m, 1H), 7.58 (d, J = 7.3 Hz, 1H), 7.50-7.46 (m, |
| | | | | 2H), 7.00-6.93 (m, 2H), 6.92-6.86 (m, 1H), 6.37 (t, J = |
| | | | | 7.1 Hz, 1H), 5.36 (dd, J = 12.6, 5.4 Hz, 1H), 3.56 |
| | | | | (s, 3H), 3.55 (s, 3H), 3.00-2.92 (m, 2H), 2.91-2.84 |
| | | | | (m, 1H), 2.80 (d, J = 4.5 Hz, 3H), 2.77-2.58 (m, 2H), |
| | | | | 2.52-2.49 (m, 2H), 2.05-1.94 (m, 1H), 1.72-1.62 (m, |
| | | | | 4H) |
| K182 | N | X | 816.4 | 11.06 (s, 1H), 10.68 (s, 1H), 10.08 (s, 1H), 9.07-9.02 |
| | | | | (m, 2H), 8.67-8.63 (m, 1H), 8.60-8.50 (m, 2H), 8.43- |
| | | | | 8.35 (m, 1H), 8.08-8.01 (m, 1H), 7.98 (s, 1H), 7.76- |
| | | | | 7.71 (m, 1H), 7.67-7.62 (m, 1H), 7.46-7.40 (m, 1H), |
| | | | | 7.37-7.30 (m, 1H), 7.01-6.93 (m, 2H), 6.92-6.86 (m, |
| | | | | 1H), 5.36 (dd, J = 12.5, 5.1 Hz, 1H), 3.70 (s, 3H), |
| | | | | 3.56 (s, 3H), 3.35-3.33 (m, 2H), 3.30-3.26 (m, 2H), |
| | | | | 3.00-2.80 (m, 3H), 2.76-2.58 (m, 2H), 2.04-1.93 (m, |
| | | | | 1H), 1.72-1.58 (m, 4H), 1.20-1.11 (m, 3H) |
| K181 | N | EN | 816.4 | 11.08 (s, 1H), 10.69 (s, 1H), 9.92 (s, 1H), 9.08-9.02 |
| | | | | (m, 2H), 8.62 (t, J = 5.6 Hz, 1H), 8.58-8.51 (m, 2H), |
| | | | | 8.27 (d, J = 5.2 Hz, 1H), 7.98 (s, 1H), 7.89 (s, 1H), |
| | | | | 7.73 (dd, J = 8.0, 1.7 Hz, 1H), 7.44-7.38 (m, 1H), |
| | | | | 7.33 (t, J = 7.9 Hz, 1H), 7.18 (dd, J = 5.3, 1.5 Hz, |
| | | | | 1H), 6.99-6.95 (m, 2H), 6.93-6.88 (m, 1H), 5.37 (dd, |
| | | | | J = 12.5, 5.4 Hz, 1H), 3.70 (s, 3H), 3.56 (s, 3H), |
| | | | | 3.37-3.33(m, 2H), 3.31-3.26 (m, 2H), 2.99-2.90 (m, |
| | | | | 2H), 2.90-2.83 (m, 1H), 2.79-2.57 (m, 2H), 2.05-1.95 |
| | | | | (m, 1H), 1.72-1.60 (m, 4H), 1.16 (t, J = 7.2 Hz, 3H) |
| K131 | V | M | 843.4 | 11.07 (s, 1H), 10.70 (s, 1H), 10.01 (s, 1H), 9.03 (s, |
| | | | | 2H), 8.55-8.51 (m, 2H), 8.27 (d, J = 2.3 Hz, 1H), |
| | | | | 7.94 (s, 1H), 7.79-7.71 (m, 2H), 7.68-7.64 (m, 1H), |
| | | | | 7.42 (dd, J = 7.8, 1.7 Hz, 1H), 7.34 (t, J = 7.8 Hz, |
| | | | | 1H), 6.96-6.85 (m, 3H), 5.35 (dd, J = 12.7, 5.4 Hz, |
| | | | | 1H), 3.70 (s, 3H), 3.67 (s, 3H), 3.18-3.14 (m, 2H), |
| | | | | 2.94 (s, 3H), 2.91-2.55 (m, 9H), 2.16-1.94 (m, 3H), |
| | | | | 1.81-1.74 (m, 2H) |
| K180 | N | DT | 828.4 | 11.08 (s, 1H), 10.65 (s, 1H), 10.09 (s, 1H), 9.08-9.01 |
| | | | | (m, 2H), 8.67-8.63 (m, 1H), 8.57-8.52 (m, 1H), 8.49 |
| | | | | (s, 1H), 8.43-8.37 (m, 1H), 8.05 (dd, J = 8.8, 2.4 Hz, |
| | | | | 1H), 7.97 (s, 1H), 7.73 (dd, J = 8.0, 1.7 Hz, 1H), 7.64 |
| | | | | (d, J = 8.8 Hz, 1H), 7.43 (dd, J = 7.8, 1.6 Hz, 1H), |
| | | | | 7.34 (t, J = 7.9 Hz, 1H), 7.01-6.93 (m, 2H), 6.92-6.86 |
| | | | | (m, 1H), 5.36 (dd, J = 12.5, 5.4 Hz, 1H), 3.72 (s, 3H), |
| | | | | 3.56 (s, 3H), 3.31-3.29 (m, 2H), 2.99-2.91 (m, 3H), |
| | | | | 2.91-2.82 (m, 1H), 2.77-2.58 (m, 2H), 2.05-1.93 (m, |
| | | | | 1H), 1.73-1.56 (m, 4H), 0.76-0.69 (m, 2H), 0.65-0.58 |
| | | | | (m, 2H) |
| K179 | N | DJ | 828.4 | 11.08 (s, 1H), 10.65 (s, 1H), 9.92 (s, 1H), 9.06-9.02 |
| | | | | (m, 2H), 8.65-8.58 (m, 1H), 8.51 (d, J = 4.1 Hz, 1H), |
| | | | | 8.49 (s, 1H), 8.27 (d, J = 5.2 Hz, 1H), 7.99 (s, 1H), |
| | | | | 7.87 (s, 1H), 7.76-7.70 (m, 1H), 7.44-7.39 (m, 1H), |
| | | | | 7.36-7.30 (m, 1H), 7.18 (dd, J = 5.2, 1.5 Hz, 1H), |
| | | | | 7.00-6.94 (m, 2H), 6.93-6.87 (m, 1H), 5.37 (dd, J = |
| | | | | 12.5, 5.4 Hz, 1H), 3.72 (s, 3H), 3.56 (s, 3H), 3.31- |
| | | | | 3.30 (m, 2H), 2.98-2.91 (m, 3H), 2.91-2.82 (m, 1H), |
| | | | | 2.77-2.57 (m, 2H), 2.05-1.96 (m, 1H), 1.70-1.61 (m, |
| | | | | 4H), 0.74-0.68 (m, 2H), 0.66-0.59 (m, 2H) |
| K178 | W | M | 843.4 | 11.05 (s, 1H), 10.69 (s, 1H), 10.00 (s, 1H), 9.03 (s, |
| | | | | 2H), 8.53 (d, J = 9.3 Hz, 2H), 8.27 (d, J = 2.3 Hz, |
| | | | | 1H), 7.92 (s, 1H), 7.78-7.69 (m, 2H), 7.66 (d, J = 8.7 |
| | | | | Hz, 1H), 7.46-7.39 (m, 1H), 7.34 (t, J = 7.9 Hz, 1H), |
| | | | | 6.93 (d, J = 8.5 Hz, 1H), 6.85 (s, 1H), 6.65 (d, J = 8.3 |
| | | | | Hz, 1H), 5.33-5.24 (m, 1H), 3.71-3.64 (m, 5H), 3.29- |
| | | | | 3.24 (m, 5H) 2.93-2.86 (m, 4H), 2.80 (d, J = 4.4 Hz, |
| | | | | 3H), 2.70-2.57 (m, 3H), 2.03-1.89 (m, 3H), 1.80-1.72 |
| | | | | (m, 2H) |
| K169 | V | X | 857.4 | 11.08 (s, 1H), 10.68 (s, 1H), 10.03 (s, 1H), 9.05-9.01 |
| | | | | (m, 2H), 8.60-8.55 (m, 1H), 8.54 (s, 1H), 8.30-8.24 |
| | | | | (m, 1H), 7.97 (s, 1H), 7.79-7.69 (m, 2H), 7.68-7.61 |
| | | | | (m, 1H), 7.44-7.35 (m, 1H), 7.38-7.30 (m, 1H), 7.02- |
| | | | | 6.81 (m, 3H), 5.35 (dd, J = 12.7, 5.4 Hz, 1H), 3.70 (s, |
| | | | | 3H), 3.67 (s, 3H), 3.38-3.34 (m, 2H), 3.30-3.27 (m, |
| | | | | 4H), 3.20-3.12 (m, 1H), 2.94 (s, 3H), 2.92-2.83 (m, |
| | | | | 1H), 2.79-2.59 (m, 2H), 2.14-1.94 (m, 3H), 1.84-1.72 |
| | | | | (m, 2H), 1.16 (t, J = 7.2 Hz, 3H) |
| K152 | W | X | 857.3 | 11.05 (s, 1H), 10.68 (s, 1H), 10.02 (s, 1H), 9.03 (s, |
| | | | | 2H), 8.58-8.53 (m, 2H), 8.27 (d, J = 2.4 Hz, 1H), |
| | | | | 7.96 (s, 1H), 7.80-7.69 (m, 2H), 7.65 (d, J = 8.7 Hz, |
| | | | | 1H), 7.44-7.38 (m, 1H), 7.37-7.28 (m, 1H), 6.93 (d, J = |
| | | | | 8.5 Hz, 1H), 6.85 (s, 1H), 6.65 (d, J = 8.5 Hz, 1H), |
| | | | | 5.34-5.24 (m, 1H), 3.72-3.65 (m, 5H), 3.35-3.31 (m, |
| | | | | 4H), 3.30 (s, 3H), 2.91-2.87 (m, 4H), 2.74-2.55 (m, |
| | | | | 4H), 2.04-1.90 (m, 3H), 1.77 (d, J = 9.6 Hz, 2H), |
| | | | | 1.18-1.13 (m, 3H) |
| K133 | GW | R | 747.3 | 11.04 (s, 1H), 10.38 (s, 1H), 9.93 (s, 1H), 8.50-8.42 |
| | | | | (m, 2H), 8.22 (s, 1H), 7.81 (s, 1H), 7.76-7.70 (m, |
| | | | | 1H), 7.67-7.60 (m, 1H), 7.54-7.49 (m, 1H), 7.13-7.08 |
| | | | | (m, 2H), 7.06-6.99 (m, 1H), 6.97-6.91 (m, 1H), 6.88- |
| | | | | 6.83 (m, 1H), 6.69-6.62 (m, 1H), 5.29 (dd, J = 13.1, |
| | | | | 5.5 Hz, 1H), 3.85 (s, 3H), 3.71-3.60 (m, 2H), 3.35 (s, |
| | | | | 3H), 3.31-3.29 (m, 2H), 3.29-3.28 (m, 1H), 2.90-2.87 |
| | | | | (m, 4H), 2.79 (d, J = 4.4 Hz, 3H), 2.72-2.59 (m, 2H), |
| | | | | 2.02-1.91 (m, 3H), 1.81-1.72 (m, 2H) |
| K370 | BA | I | 977.5 | 11.10 (s, 1H), 9.30 (t, J = 5.9 Hz, 1H), 9.10-9.04 (m, |
| | | | | 1H), 8.69-8.62 (m, 2H), 8.44 (dd, J = 8.5, 2.4 Hz, |
| | | | | 1H), 8.05-7.97 (m, 2H), 7.93 (s, 1H), 7.59 (dd, J = |
| | | | | 7.1, 1.7 Hz, 1H), 7.54-7.48 (m, 1H), 7.31-7.17 (m, |
| | | | | 3H), 7.17-7.08 (m, 1H), 7.07-7.02 (m, 1H), 7.01- |
| | | | | 6.94 (m, 2H), 6.44 (t, J = 7.2 Hz, 1H), 6.40 (s, 1H), |
| | | | | 5.37 (dd, J = 12.8, 5.3 Hz, 1H), 4.99-4.76 (m, 1H), |
| | | | | 4.44 (d, J = 5.8 Hz, 2H), 3.97-3.89 (m, 1H), 3.86- |
| | | | | 3.77 (m, 2H), 3.73-3.65 (m, 1H), 3.48-3.41 (m, 4H), |
| | | | | 3.36 (s, 3H), 3.31-3.21 (m, 1H), 3.06-2.96 (m, 1H), |
| | | | | 2.96-2.90 (m, 1H), 2.88 (d, J = 4.7 Hz, 3H), 2.77- |
| | | | | 2.60 (m, 4H), 2.47-2.37 (m, 1H), 2.23-2.10 (m, 2H), |
| | | | | 2.06-1.97 (m, 1H), 1.81-1.68 (m, 2H), 1.29-1.16 (m, |
| | | | | 1H), 1.06-0.92 (m, 1H) |
| K371 | AV | I | 946.5 | 11.13 (s, 1H), 8.70 (s, 1H), 8.65-8.60 (m, 2H), 8.54- |
| | | | | 8.50 (m, 2H), 8.16-8.10 (m, 1H), 7.99 (d, J = 7.7 Hz, |
| | | | | 2H), 7.92 (s, 1H), 7.60 (d, J = 7.0 Hz, 1H), 7.50-7.44 |
| | | | | (m, 1H), 7.37 (s, 1H), 7.27-7.20 (m, 1H), 7.20-7.14 |
| | | | | (m, 1H), 6.47-6.39 (m, 1H), 6.37 (s, 1H), 5.45-5.36 |
| | | | | (m, 1H), 4.98-4.65 (m, 1H), 4.01-3.56 (m, 6H), 3.45- |
| | | | | 3.36 (m, 5H), 3.06-2.97 (m, 1H), 2.93-2.87 (m, 4H), |
| | | | | 2.81-2.59 (m, 2H), 2.10-2.05 (m, 1H), 1.98-1.87 (m, |
| | | | | 2H), 1.62-1.58 (m, 2H), 1.30-1.18 (m, 1H), 1.07-0.94 |
| | | | | (m, 1H) |
| K372 | FQ | I | 892.3 | 11.09 (s, 1H), 8.88 (s, 1H), 8.70-8.61 (m, 2H), 8.27 |
| | | | | (dd, J = 8.4, 2.4 Hz, 1H), 7.99 (dd, J = 6.6, 4.1 Hz, |
| | | | | 2H), 7.92 (s, 1H), 7.62-7.57 (m, 1H), 7.55-7.46 (m, |
| | | | | 3H), 7.40-7.34 (m, 1H), 7.27-7.21 (m, 1H), 7.12 (d, J = |
| | | | | 8.2 Hz, 1H), 6.54 (d, J = 8.2 Hz, 2H), 6.48-6.34 |
| | | | | (m, 2H), 5.35 (d, J = 5.6 Hz, 1H), 4.99-4.76 (m, 1H), |
| | | | | 4.64 (s, 2H), 4.33 (s, 2H), 4.09-3.97 (m, 4H), 3.41 (s, |
| | | | | 3H), 3.03-2.95 (m, 1H), 2.87 (d, J = 4.8 Hz, 3H), |
| | | | | 2.81-2.59 (m, 3H), 2.08-1.99 (m, 1H), 1.25-1.20 (m, |
| | | | | 1H), 1.04-0.92 (m, 1H) |
| K373 | DC | I | 948.7 | 11.11 (s, 1H), 8.70-8.62 (m, 3H), 8.12-8.05 (m, 1H), |
| | | | | 8.01 (d, J = 7.5 Hz, 1H), 7.97 (d, J = 8.2 Hz, 1H), |
| | | | | 7.92 (s, 1H), 7.66-7.57 (m, 3H), 7.52-7.46 (m, 2H), |
| | | | | 7.40-7.23 (m, 3H), 7.18 (d, J = 8.2 Hz, 1H), 6.44 (t, J = |
| | | | | 7.2 Hz, 1H), 6.40 (s, 1H), 5.40 (dd, J = 12.8, 5.4 |
| | | | | Hz, 1H), 4.98-4.78 (m, 1H), 3.75-3.63 (m, 2H), 3.41 |
| | | | | (s, 3H), 3.36-3.31 (m, 2H), 3.08-2.92 (m, 2H), 2.88 |
| | | | | (d, J = 4.8 Hz, 3H), 2.83-2.57 (m, 2H), 2.53-2.47 (m, |
| | | | | 8H), 2.27-2.16 (m, 2H), 2.06-1.97 (m, 1H), 1.83-1.76 |
| | | | | (m, 2H), 1.29-1.21 (m, 1H), 1.05-0.94 (m, 1H) |
| K374 | AA | I | 977.6 | 11.10 (s, 1H), 8.71-8.52 (m, 3H), 8.10-8.07 (m, 1H), |
| | | | | 8.04-7.96 (m, 2H), 7.92 (s, 1H), 7.64-7.59 (m, 1H), |
| | | | | 7.55-7.46 (m, 3H), 7.41-7.39 (m, 1H), 7.30-7.23 (m, |
| | | | | 1H), 7.14 (d, J = 8.3 Hz, 1H), 7.02-6.98 (m, 2H), |
| | | | | 6.48-6.38 (m, 2H), 5.43-5.34 (m, 1H), 5.01-4.79 (m, |
| | | | | 1H), 3.85-3.61 (m, 5H), 3.47-3.35 (m, 4H), 3.05-2.99 |
| | | | | (m, 1H), 2.94-2.87 (m, 4H), 2.77-2.61 (m, 4H), 2.49- |
| | | | | 2.38 (m, 4H), 2.26-2.20 (m, 2H), 2.10-2.00 (m, 1H), |
| | | | | 1.86-1.78 (m, 2H), 1.71 (s, 1H), 1.32-1.18 (m, 3H), |
| | | | | 1.07-0.92 (m, 1H) |
| K375 | HA | I | 1015.5 | 11.13 (s, 1H), 8.71-8.56 (m, 5H), 8.05 (dd, J = 8.3, |
| | | | | 2.3 Hz, 1H), 8.00 (dd, J = 7.3, 1.8 Hz, 1H), 7.96 (d, J = |
| | | | | 8.3 Hz, 1H), 7.92 (s, 1H), 7.59 (dd, J = 7.1, 1.7 Hz, |
| | | | | 1H), 7.52-7.48 (m, 1H), 7.38 (d, J = 1.5 Hz, 1H), |
| | | | | 7.25 (dd, J = 8.1, 1.6 Hz, 1H), 7.19 (d, J = 8.3 Hz, |
| | | | | 1H), 6.44 (t, J = 7.2 Hz, 1H), 6.37 (s, 1H), 5.40 (dd, J = |
| | | | | 12.9, 5.3 Hz, 1H), 4.97-4.77 (m, 1H), 4.66-4.46 (m, |
| | | | | 3H), 4.32 (d, J = 11.1 Hz, 2H), 3.97-3.89 (m, 2H), |
| | | | | 3.74-3.48 (m, 1H), 3.55-3.45 (m, 2H), 3.29-3.12 (m, |
| | | | | 3H), 3.07-2.92 (m, 1H), 2.93-2.84 (m, 4H), 2.78-2.57 |
| | | | | (m, 3H), 2.46-2.40 (m, 1H), 2.10-2.00 (m, 1H), 1.94- |
| | | | | 1.71 (m, 4H), 1.31-1.21 (m, 4H), 1.05-0.95 (m, 1H) |
| K376 | FI | I | 918.4 | 11.06 (s, 1H), 8.90 (d, J = 2.3 Hz, 1H), 8.66-8.62 (m, |
| | | | | 2H), 8.57 (s, 2H), 8.29 (dd, J = 8.4, 2.4 Hz, 1H), |
| | | | | 8.03-7.99 (m, 2H), 7.92 (s, 1H), 7.61 (dd, J = 7.2, 1.8 |
| | | | | Hz, 1H), 7.52-7.48 (m, 1H), 7.41 (s, 1H), 7.26 (dd, J = |
| | | | | 8.1, 1.6 Hz, 1H), 7.19 (d, J = 8.3 Hz, 1H), 6.44 (t, J = |
| | | | | 7.2 Hz, 1H), 6.39 (s, 1H), 5.41 (dd, J = 12.7, 5.4 |
| | | | | Hz, 1H), 4.99-4.79 (m, 1H), 4.90-4.87 (m, 1H), 4.37 |
| | | | | (t, J = 7.4 Hz, 2H), 4.31-4.27 (m, 2H), 3.38 (s, 3H), |
| | | | | 3.04-2.93 (m, 2H), 2.88 (d, J = 4.9 Hz, 3H), 2.81- |
| | | | | 2.59 (m, 5H), 2.10-2.03 (m, 1H), 1.33-1.19 (m, 1H), |
| | | | | 1.08-0.92 (m, 1H) |
| K377 | FS | I | 962.4 | 11.11 (s, 1H), 8.69 (d, J = 2.2 Hz, 1H), 8.66-8.62 (m, 2H), |
| | | | | 8.10 (dd, J = 8.3, 2.3 Hz, 1H), 8.04-7.94 (m, 2H), 7.92 (s, |
| | | | | 1H), 7.65-7.56 (m, 3H), 7.54-7.44 (m, 2H), 7.37-7.29 (m, |
| | | | | 3H), 7.19 (d, J = 8.2 Hz, 1H), 6.44 (t, J = 7.2 Hz, 1H), |
| | | | | 6.40-6.38 (m, 1H), 5.40 (dd, J = 12.8, 5.4 Hz, 1H), |
| | | | | 4.98-4.77 (m, 1H), 3.78-3.67 (m, 2H), 3.56-3.44 (m, |
| | | | | 4H), 3.41 (s, 3H), 3.04-2.91 (m, 2H), 2.88 (d, J = 4.8 |
| | | | | Hz, 3H), 2.83-2.60 (m, 4H), 2.59-2.55 (m, 1H), 2.34- |
| | | | | 2.27 (m, 1H), 2.10-2.02 (m, 1H), 2.01-1.84 (m, 4H), |
| | | | | 1.66-1.48 (m, 4H), 1.29-1.22 (m, 1H), 1.06-0.93(m, |
| | | | | 1H) |
| K378 | DD | I | 895.4 | 11.12 (s, 1H), 8.83-8.80 (m, 2H), 8.66-8.63 (m, 2H), |
| | | | | 8.26-8.13 (m, 2H), 8.04-8.00 (m, 3H), 7.93 (s, 1H), |
| | | | | 7.68-7.56 (m, 2H), 7.53-7.40 (m, 2H), 7.25 (d, J = |
| | | | | 8.3 Hz, 1H), 6.45 (t, J = 7.2 Hz, 1H), 6.40 (s, 1H), |
| | | | | 5.42 (dd, J = 12.8, 5.4 Hz, 1H), 4.99-4.79 (m, 1H), |
| | | | | 4.51-4.37 (m, 2H), 4.19 (s, 2H), 4.05-3.86(m, 2H), |
| | | | | 3.43 (s, 3H), 3.04-2.98 (m, 1H), 2.95-2.89 (m, 1H), |
| | | | | 2.89 (d, J = 4.8 Hz, 3H), 2.83-2.62 (m, 2H), 2.13- |
| | | | | 1.96 (m, 1H), 1.31-1.19 (m, 1H), 1.05-0.95 (m, 1H) |
| K379 | FU | I | 966.4 | 11.11 (s, 1H), 8.69-8.58 (m, 3H), 8.11-7.98 (m, 2H), |
| | | | | 7.98-7.88 (m, 2H), 7.65-7.55 (m, 3H), 7.52-7.43 (m, |
| | | | | 2H), 7.36-7.25(m, 3H), 7.18 (d, J = 8.2 Hz, 1H), |
| | | | | 6.48-6.35 (m, 2H), 5.40 (dd, J = 12.7, 5.4 Hz, 1H), |
| | | | | 5.01-5.76 (m, 1H), 3.70-3.56 (m, 2H), 3.50-3.43 (m, |
| | | | | 2H), 3.41 (s, 3H), 3.38-3.34 (m, 6H), 3.05-2.96 (m, |
| | | | | 1H), 2.94-2.80 (m, 4H), 2.78-2.58 (m, 2H), 2.47-2.27 |
| | | | | (m, 6H), 2.11-1.97 (m, 1H), 1.75-1.60 (m, 2H), 1.32- |
| | | | | 1.17 (m, 1H), 1.06-0.90 (m, 1H) |
| K380 | AC | I | 962.5 | 11.12 (s, 1H), 8.75 (s, 1H), 8.66 (d, J = 2.5 Hz, 2H), |
| | | | | 8.17-8.13 (m, 1H), 8.05-7.96 (m, 2H), 7.93 (s, 1H), |
| | | | | 7.64-7.58 (m, 3H), 7.54-7.48 (m, 1H), 7.47 (d, J = |
| | | | | 1.6 Hz, 1H), 7.35-7.31 (m, 3H), 7.18 (d, J = 8.3 Hz, |
| | | | | 1H), 6.46 (t, J = 7.2 Hz, 1H), 6.40 (s, 1H), 5.40 (dd, J = |
| | | | | 12.8, 5.4 Hz, 1H), 4.98-4.78 (m, 1H), 3.41 (s, 3H), |
| | | | | 3.22-2.91 (m, 6H), 2.89 (d, J = 4.8 Hz, 3H), 2.82- |
| | | | | 2.56 (m, 2H), 2.54-2.51 (m, 4H), 2.21-1.84 (m, 5H), |
| | | | | 1.65-1.52 (m, 4H), 1.39-1.08 (m, 2H), 1.05-0.83 (m, |
| | | | | 2H) |
| K381 | AD | I | 942.5 | 11.03 (s, 1H), 8.68-8.64 (m, 3H), 8.11-8.05 (m, 1H), |
| | | | | 8.02-7.98 (m, 1H), 7.96-7.92 (m, 1H), 7.92 (s, 1H), |
| | | | | 7.62-7.57 (m, 1H), 7.53-7.48 (m, 1H), 6.94 (d, J = |
| | | | | 8.6 Hz, 1H), 6.84 (d, J = 2.1 Hz, 1H), 6.65-6.61 (m, |
| | | | | 1H), 6.46-6.42 (m, 1H), 6.40 (s, 1H), 5.31-5.27 (m, |
| | | | | 1H), 4.98-4.76 (m, 1H), 4.09-4.05 (m, 1H), 3.62-3.46 |
| | | | | (m, 3H), 3.47-3.36 (m, 2H), 3.27-3.15 (m, 2H), 3.15- |
| | | | | 3.01 (m, 4H), 3.02-2.95(m, 1H), 2.93-2.82 (m, 8H), |
| | | | | 2.65-2.55 (m, 2H), 2.44-2.29 (m, 5H), 2.02-1.95 (m, |
| | | | | 1H), 1.79-1.59 (m, 2H), 1.30-1.20 (m, 3H), 1.04-0.94 |
| | | | | (m, 1H) |
| K382 | BO | I | 918.4 | 11.13 (s, 1H), 8.87 (d, J = 2.4 Hz, 1H), 8.70-8.61 (m, |
| | | | | 2H), 8.54 (s, 2H), 8.26 (dd, J = 8.4, 2.4 Hz, 1H), |
| | | | | 8.04-7.97 (m, 2H), 7.92 (s, 1H), 7.61 (dd, J = 7.1, 1.7 |
| | | | | Hz, 1H), 7.54-7.49 (m, 1H), 7.39 (d, J = 1.5 Hz, 1H), |
| | | | | 7.27-7.22 (m, 1H), 7.21-7.14 (m, 1H), 6.52-6.35 (m, |
| | | | | 2H), 5.41 (dd, J = 12.6, 5.4 Hz, 1H), 5.06-4.74 (m, |
| | | | | 1H), 4.65 (s, 2H), 4.39-4.25 (m, 6H), 3.31 (s, 3H), |
| | | | | 3.07-2.95 (m, 1H), 2.93-2.83 (m, 4H), 2.80-2.61 (m, |
| | | | | 2H), 2.10-1.99 (m, 1H), 1.27-1.21 (m, 1H), 1.12- |
| | | | | 0.92 (m, 1H) |
| K195 | V | R | 747.4 | 11.09 (s, 1H), 10.39 (s, 1H), 9.96 (s, 1H), 8.49-8.45 |
| | | | | (m, 2H), 8.22 (s, 1H), 7.82 (s, 1H), 7.76-7.72 (m, |
| | | | | 1H), 7.63 (d, J = 8.6 Hz, 1H), 7.54-7.50 (m, 1H), |
| | | | | 7.13-7.00 (m, 3H), 6.97-6.87 (m, 3H), 5.37-5.33 (m, |
| | | | | 1H), 3.85 (s, 3H), 3.67 (s, 3H), 3.18-3.14 (m, 2H), |
| | | | | 2.93 (s, 3H), 2.91-2.85 (m, 5H), 2.79 (d, J = 4.5 Hz, |
| | | | | 3H), 2.71-2.59 (m, 2H), 2.07-1.99 (m, 3H), 1.83-1.70 |
| | | | | (m, 2H) |
| K383 | GY | I | 921.1 | 11.07 (s, 1H), 8.90 (t, J = 1.4 Hz, 1H), 8.65-8.63 (m, |
| | | | | 2H), 8.47 (d, J = 2.6 Hz, 1H), 8.30 (dd, J = 8.5, 2.3 |
| | | | | Hz, 1H), 8.02-7.98 (m, 2H), 7.93 (d, J = 1.2 Hz, 1H), |
| | | | | 7.85 (dd, J = 8.9, 2.6 Hz, 1H), 7.60 (dd, J = 7.1, 1.7 |
| | | | | Hz, 1H), 7.49 (q, J = 4.9 Hz, 1H), 7.45 (d, J = 1.7 |
| | | | | Hz, 1H), 7.28 (dd, J = 8.3, 1.7 Hz, 1H), 7.16 (d, J = |
| | | | | 8.2 Hz, 1H), 6.95 (d, J = 8.9 Hz, 1H), 6.44 (t, J = 7.2 |
| | | | | Hz, 1H), 6.39 (s, 1H), 5.39 (dd, J = 12.8, 5.4 Hz, |
| | | | | 1H), 4.98-4.78 (m, 1H), 4.22 (s, 2H), 3.89 (s, 2H), |
| | | | | 3.70-3.59 (m, 2H), 3.56-3.46 (m, 2H), 3.40 (s, 3H), |
| | | | | 3.10-2.91 (m, 2H), 2.88 (d, J = 4.8 Hz, 3H), 2.81- |
| | | | | 2.61 (m, 2H), 2.06 (dd, J = 11.4, 5.6 Hz, 1H), 1.87- |
| | | | | 1.76 (m, 4H), 1.30-1.21 (m, 1H), 10.6-0.95 (m, 1H) |
| K384 | FY | I | 993.4 | 11.02 (s, 1H), 9.01 (d, J = 2.3 Hz, 1H), 8.67 (s, 1H), |
| | | | | 8.64 (d, J = 4.3 Hz, 1H), 8.57 (d, J = 7.6 Hz, 1H), |
| | | | | 8.39 (dd, J = 8.4, 2.4 Hz, 1H), 8.04-7.95 (m, 2H), |
| | | | | 7.92 (s, 1H), 7.60-7.57 (m, 1H), 7.53-7.47 (m, 1H), |
| | | | | 7.32 (s, 1H), 7.23-7.06 (m, 2H), 6.47-6.42 (m, 1H), |
| | | | | 6.41 (s, 1H), 5.42-5.36 (m, 1H), 4.99-4.79 (m, 1H), |
| | | | | 3.81-3.75 (m, 1H), 3.65 (s, 1H), 3.56-3.51 (m, 1H), |
| | | | | 3.49-3.46 (m, 2H), 3.42-3.39 (m, 1 H), 3.36 (s, 3H), |
| | | | | 3.29-3.21 (m, 5H), 3.09-2.95 (m, 1H), 2.94-2.82 (m, |
| | | | | 4H), 2.79-2.57 (m, 2H), 2.40-2.31 (m, 1H), 2.15-1.99 |
| | | | | (m, 2H), 1.99-1.88 (m, 3H), 1.86-1.68 (m, 2H), 1.52- |
| | | | | 1.37 (m, 4H), 1.32-1.15 (m, 1H), 1.06-0.96 (m, 1H) |
| K385 | FW | I | 881.4 | 11.10 (s, 1H), 9.14-9.09 (m, 2H), 8.71 (d, J = 2.2 Hz, |
| | | | | 1H), 8.67-8.61 (m, 2H), 8.13 (dd, J = 8.3, 2.3 Hz, |
| | | | | 1H), 8.03-7.94 (m, 2H), 7.92 (s, 1H), 7.70-7.65 (m, |
| | | | | 1H), 7.61 (dd, J = 7.1, 1.7 Hz, 1H), 7.52-7.45 (m, |
| | | | | 2H), 7.28 (d, J = 8.2 Hz, 1H), 6.48-6.41 (m, 1H), |
| | | | | 6.40-6.36 (m, 1H), 5.43 (dd, J = 12.8, 5.4 Hz, 1H), |
| | | | | 4.99-4.77 (m, 1H), 4.67-4.54 (m, 1H), 3.81-3.69 (m, |
| | | | | 1H), 3.42 (s, 3H), 3.40-3.38 (m, 1H), 3.28-3.20 (m, |
| | | | | 1H), 3.15-3.05 (m, 1H), 3.04-2.91 (m, 2H), 2.88 (d, J = |
| | | | | 4.9 Hz, 3H), 2.83-2.73 (m, 1H), 2.70-2.62 (m, 1H), |
| | | | | 2.22-2.01 (m, 3H), 1.94-1.79 (m, 2H), 1.33-1.19 (m, |
| | | | | 1H), 1.06-0.92 (m, 1H) |
| K386 | FD | I | 832.4 | 11.09 (s, 1H), 8.72-8.60 (m, 3H), 8.16-8.05 (m, 1H), |
| | | | | 8.04-7.89 (m, 3H), 7.63-7.56 (m, 1H), 7.55-7.47 (m, |
| | | | | 1H), 7.09 (s, 1H), 7.05-6.97 (m, 1H), 6.96-6.87 (m, |
| | | | | 1H), 6.50-6.33 (m, 2H), 5.34 (dd, J = 12.7, 5.4 Hz, |
| | | | | 1H), 5.01-4.73 (m, 1H), 3.78-3.57 (m, 2H), 3.48-3.39 |
| | | | | (m, 2H), 3.36(s, 3H), 3.04-2.96 (m, 1H), 2.88 (d, J = |
| | | | | 4.7 Hz, 3H), 2.84-2.75 (m, 2H), 2.75-2.57 (m, 5H), |
| | | | | 2.50-2.48 (m, 4H), 2.05-1.92 (m, 1H), 1.31-1.18 (m, |
| | | | | 1H), 1.05-0.91 (m, 1H) |
| K387 | HC | I | 840.4 | 11.13 (s, 1H), 9.01-8.91 (m, 1H), 8.82-8.78 (m, 1H), |
| | | | | 8.70-8.57 (m, 2H), 8.31-8.08 (m, 2H), 8.05-7.86 (m, |
| | | | | 3H), 7.69-7.37 (m, 5H), 7.29-7.18 (m, 1H), 6.50-6.30 |
| | | | | (m, 2H), 5.43 (dd, J = 12.8, 5.4 Hz, 1H), 5.01-4.75 |
| | | | | (m, 2H), 4.71-4.63 (m, 1H), 3.43 (s, 3H), 3.14-2.91 |
| | | | | (m, 5H), 2.90-2.83 (m, 3H), 2.82-2.60 (m, 2H), 2.11- |
| | | | | 1.98 (m, 1H), 1.31-1.13 (m, 1H), 1.06-0.89 (m, 1H) |
| K388 | CN | I | 844.4 | 11.06 (s, 1H), 8.90 (s, 1H), 8.69-8.62 (m, 2H), 8.32- |
| | | | | 8.28 (m, 1H), 8.03-7.97 (m, 2H), 7.92 (s, 1H), 7.62- |
| | | | | 7.57 (m, 1H), 7.55-7.49 (m, 1H), 6.94 (d, J = 8.5 Hz, |
| | | | | 1H), 6.86 (s, 1H), 6.68-6.62 (m, 1H), 6.48-6.37 (m, |
| | | | | 2H), 5.29 (dd, J = 12.9, 5.4 Hz, 1H), 5.01-4.75 (m, |
| | | | | 1H), 4.21 (s, 2H), 3.88 (s, 2H), 3.31(s, 3H), 3.18-3.08 |
| | | | | (m, 1H), 3.08-2.95 (m, 3H), 2.87 (d, J = 5.0 Hz, 3H), |
| | | | | 2.77-2.56 (m, 2H), 2.49-2.45(m, 2H), 2.02-1.94 (m, |
| | | | | 1H), 1.94-1.85 (m, 4H), 1.06-0.92 (m, 1H), 0.89-0.79 |
| | | | | (m, 1H) |
| K389 | AE | I | 844.4 | 11.09 (s, 1H), 8.91 (s, 1H), 8.72-8.62 (m, 2H), 8.33- |
| | | | | 8.29 (m, 1H), 8.03-7.98 (m, 2H), 7.93 (s, 1H), 7.60 |
| | | | | (d, J = 7.2 Hz, 1H), 7.54-7.50 (m, 1H), 7.01-6.85 (m, |
| | | | | 3H), 6.53-6.36 (m, 2H), 5.35 (dd, J = 12.7, 5.4 Hz, |
| | | | | 1H), 4.97-4.78 (m, 1H), 4.30-4.17 (m, 2H), 3.99-3.86 |
| | | | | (m, 2H), 3.64 (s, 3H), 3.07-3.00 (m, 3H), 2.91-2.84 |
| | | | | (m, 4H), 2.79-2.56 (m, 3H), 2.13-1.79 (m, 6H), 1.29- |
| | | | | 1.20 (m, 1H), 1.04-0.95 (m, 1H) |
| K391 | EX | I | 857.5 | 11.08 (s, 1H), 8.71-8.56 (m, 3H), 8.07-7.98 (m, 2H), |
| | | | | 7.96-7.88 (m, 2H), 7.61-7.57 (m, 1H), 7.54-7.48 (m, |
| | | | | 1H), 7.03-6.94 (m, 2H), 6.87-6.81 (m, 1H), 6.46-6.36 |
| | | | | (m, 2H), 5.33 (dd, J = 12.7, 5.5 Hz, 1H), 5.01-4.71 |
| | | | | (m, 1H), 3.56-3.44 (m, 4H), 3.32 (s, 3H), 3.04-2.92 |
| | | | | (m, 2H), 2.92-2.80 (m, 4H), 2.77-2.57 (m, 4H), 2.06- |
| | | | | 1.81 (m, 3H), 1.71-1.44 (m, 6H), 1.31-1.17 (m, 1H), |
| | | | | 1.08-0.91 (m, 1H) |
| K393 | AX | I | 873.4 | 11.08 (s, 1H), 8.73-8.58 (m, 3H), 8.13-8.05 (m, 1H), |
| | | | | 8.04-7.99 (m, 1H), 7.97-7.91 (m, 2H), 7.64-7.56 (m, |
| | | | | 1H), 7.54-7.50 (m, 1H), 7.14-7.06 (m, 1H), 7.05-7.00 |
| | | | | (m, 1H), 6.97-6.89 (m, 1H), 6.47-6.36 (m, 2H), 5.38- |
| | | | | 5.27 (m, 1H), 5.01-4.71 (m, 1H), 4.11-3.94 (m, 2H), |
| | | | | 3.70-3.55 (m, 2H), 3.35 (s, 3H), 3.06-2.94 (m, 2H), |
| | | | | 2.92-2.85 (m, 4H), 2.73-2.59 (m, 3H), 2.44-2.37 (m, |
| | | | | 2H), 2.08-1.75 (m, 5H), 1.58-1.48 (m, 2H), 1.33-1.18 |
| | | | | (m, 1H), 1.07-0.87 (m, 1H) |
| K706 | AQ | AR | 706.3 | 11.06 (s, 1H), 10.45-10.33 (m, 1H), 10.04-9.92 (m, |
| | | | | 1H), 8.66-8.56 (m, 1H), 8.51-8.42 (m, 2H), 8.40-8.34 |
| | | | | (m, 1H), 8.07-8.01 (m, 1H), 7.87-7.81 (m, 1H), 7.68- |
| | | | | 7.48 (m, 3H), 7.14-6.98 (m, 4H), 6.92-6.86 (m, 1H), |
| | | | | 5.38-5.15 (m, 1H), 3.91-3.78 (m, 3H), 3.33 (d, J = |
| | | | | 4.6 Hz, 3H), 2.96-2.86 (m, 1H), 2.82-2.75 (m, 3H), |
| | | | | 2.73-2.58 (m, 4H), 2.06-1.96 (m, 1H), 1.60 (d, J = |
| | | | | 37.4 Hz, 4H), 1.24 (s, 1H), 0.92-0.69 (m, 1H) |
| K710 | V | AF | 769.5 | 11.07 (s, 1H), 11.03 (s, 1H), 10.45 (s, 1H), 9.12 (s, |
| | | | | 1H), 8.34-8.15 (m, 2H), 7.84-7.80 (m, 1H), 7.66 (d, J = |
| | | | | 8.7 Hz, 1H), 7.43 (d, J = 8.2 Hz, 1H), 7.29-7.20 |
| | | | | (m, 1H), 7.12-7.03 (m, 1H), 6.99-6.84 (m, 3H), 5.43- |
| | | | | 5.29 (m, 1H), 3.89 (s, 3H), 3.67 (s, 3H), 3.18-3.09 |
| | | | | (m, 3H), 2.98-2.82 (m, 4H), 2.79-2.54 (m, 3H), 2.13- |
| | | | | 1.95 (m, 4H), 1.78 (d, J = 11.9 Hz, 2H) |
| K408 | CE | I | 900.5 | 11.08 (s, 1H), 8.67-8.64 (m, 3H), 8.07 (dd, J = 8.2, |
| | | | | 2.3 Hz, 1H), 8.01 (dd, J = 7.4, 1.8 Hz, 1H), 7.97-7.93 |
| | | | | (m, 1H), 7.92 (s, 1H), 7.63-7.58 (m, 1H), 7.56-7.47 |
| | | | | (m, 1H), 7.10 (s, 1H), 7.01 (d, J = 8.1 Hz, 1H), 6.92 |
| | | | | (d, J = 8.2 Hz, 1H), 6.47-6.38 (m, 2H), 5.34 (dd, J = |
| | | | | 12.7, 5.4 Hz, 1H), 5.02-4.73 (m, 1H), 4.57-4.51 (m, |
| | | | | 1H), 3.67-3.44 (m, 2H), 3.34 (s, 3H), 3.25-3.08 (m, |
| | | | | 2H), 3.01-2.99 (m, 3H), 2.93-2.83 (m, 4H), 2.76-2.58 |
| | | | | (m, 2H), 2.26-2.13 (m, 2H), 2.06-1.94 (m, 3H), 1.93- |
| | | | | 1.79 (m, 2H), 1.80-1.60 (m, 5H), 1.31-1.20 (m, 1H), |
| | | | | 1.18-1.08 (m, 2H), 1.07-0.90 (m, 1H) |
| K409 | FZ | I | 960.4 | 11.13 (s, 1H), 9.19 (d, J = 23.6 Hz, 2H), 8.83 (s, 1H), |
| | | | | 8.71-8.61 (m, 2H), 8.24 (d, J = 8.3 Hz, 1H), 8.05- |
| | | | | 7.05 (m, 2H), 7.92 (d, J = 2.7 Hz, 1H), 7.64-7.47 (m, |
| | | | | 3H), 7.42-7.35 (m, 1H), 7.28-7.21 (m, 1H), 6.47-6.35 |
| | | | | (m, 2H), 5.48-5.39 (m, 1H), 5.00-4.75 (m, 1H), 4.04- |
| | | | | 3.87 (m, 2H), 3.82 (d, J = 11.4 Hz, 2H), 3.77-3.64 |
| | | | | (m, 2H), 3.39 (d, J = 4.1 Hz, 3H), 3.04-2.94 (m, 1H), |
| | | | | 2.93-2.83 (m, 4H), 2.81-2.59 (m, 2H), 2.38-2.15 (m, |
| | | | | 3H), 2.13-2.01 (m, 2H), 1.31-1.17 (m, 1H), 1.06-1.92 |
| | | | | (m, 1H) |
| K410 | CQ | I | 858.4 | 11.09 (s, 1H), 8.74-8.58 (m, 3H), 8.11-8.07 (m, 1H), |
| | | | | 8.03-7.99 (m, 1H), 7.98-7.90 (m, 2H), 7.63-7.57 (m, |
| | | | | 1H), 7.54-7.48 (m, 1H), 7.18 (s, 1H), 7.08-6.99 (m, |
| | | | | 2H), 6.48-6.35 (m, 2H), 5.36 (dd, J = 12.8, 5.3 Hz, |
| | | | | 1H), 5.03-4.75 (m, 1H), 4.15-4.09 (m, 1H), 3.70-3.54 |
| | | | | (m, 4H), 3.36 (s, 3H), 3.30-3.18 (m, 2H), 3.15-3.05 |
| | | | | (m, 2H), 3.04-2.90 (m, 2H), 2.88 (d, J = 4.8 Hz, 3H), |
| | | | | 2.79-2.58 (m, 2H), 2.42-2.40 (m, 1H), 2.10-1.95 (m, |
| | | | | 1H), 1.81-1.62 (m, 2H), 1.39-1.16 (m, 3H), 1.07-0.92 |
| | | | | (m, 1H) |
| K411 | CE | I | 900.4 | 11.05 (s, 1H), 8.69-8.61 (m, 3H), 8.07 (dd, J = 8.3, |
| | | | | 2.3 Hz, 1H), 8.01 (dd, J = 7.4, 1.8 Hz, 1H), 7.95 (d, J = |
| | | | | 8.3 Hz, 1H), 7.92 (s, 1H), 7.60 (dd, J = 7.1, 1.7 Hz, |
| | | | | 1H), 7.53-7.45 (m, 1H), 6.94 (d, J = 8.6 Hz, 1H), |
| | | | | 6.84 (d, J = 2.2 Hz, 1H), 6.63 (dd, J = 8.7, 2.2 Hz, |
| | | | | 1H), 6.47-6.37 (m, 2H), 5.29 (dd, J = 12.9, 5.4 Hz, |
| | | | | 1H), 4.97-4.79 (m, 1H), 4.57-4.45 (m, 1H), 3.68-3.58 |
| | | | | (m, 1H), 3.33-3.28 (m, 5H), 3.14-3.05 (m, 4H), 3.04- |
| | | | | 2.97 (m, 1H), 2.88 (d, J = 4.9 Hz, 3H), 2.75-2.58 (m, |
| | | | | 2H), 2.56-2.53 (m, 3H), 2.28-2.20 (m, 2H), 2.04-1.96 |
| | | | | (m, 1H), 1.93-1.81 (m, 2H), 1.79-1.70 (m, 1H), 1.31- |
| | | | | 1.08 (m, 3H), 1.02-0.95 (m, 1H) |
| K416 | CF | I | 868.4 | 11.12 (s, 1H), 8.81-8.77 (m, 1H), 8.66-8.64 (m, 2H), |
| | | | | 821-8.17 (m, 1H), 8.01 (d, J = 7.3 Hz, 1H), 7.96 (d, J = |
| | | | | 8.4 Hz, 1H), 7.92 (s, 1H), 7.59 (d, J = 7.0 Hz, 1H), |
| | | | | 7.53-7.49 (m, 1H), 7.31 (d, J = 19.8 Hz, 1H), 7.22- |
| | | | | 7.06 (m, 2H), 6.46-6.38 (m, 2H), 5.43-5.35 (m, 1H), |
| | | | | 5.01-4.77 (m, 1H), 3.66 (d, J = 4.0 Hz, 2H), 3.58- |
| | | | | 3.53 (m, 2H), 3.51-3.31 (m, 7H), 3.26-3.19 (m, 2H), |
| | | | | 3.08-2.94 (m, 1H), 2.93-2.84 (m, 4H), 2.82-2.59 (m, |
| | | | | 2H), 2.13-1.98 (m, 3H), 1.30-1.20 (m, 1H), 1.08-0.92 |
| | | | | (m, 1H) |
| K418 | GA | I | 929.5 | 11.05 (s, 1H), 8.88 (s, 1H), 8.70-8.59 (m, 2H), 8.27 |
| | | | | (d, J = 8.5 Hz, 1H), 7.99 (t, J = 8.5 Hz, 2H), 7.92 (s, |
| | | | | 1H), 7.60 (d, J = 7.1 Hz, 1H), 7.55-7.47 (m, 1H), |
| | | | | 6.94 (d, J = 8.5 Hz, 1H), 6.84 (s, 1H), 6.62 (d, J = 8.7 |
| | | | | Hz, 1H), 6.48-6.35 (m, 2H), 5.34-5.24 (m, 1H), 5.00- |
| | | | | 4.75 (m, 1H), 4.53-4.35 (m, 2H), 4.26-4.15 (m, 1H), |
| | | | | 4.14-4.01 (m, 1H), 3.98-3.86 (m, 1H), 3.60-3.50 (m, |
| | | | | 1H), 3.31 (s, 3H), 3.13-3.04 (m, 4H), 3.03-2.95 (m, |
| | | | | 1H), 2.92-2.82 (m, 4H), 2.74-2.54 (m, 6H), 2.37-2.26 |
| | | | | (m, 3H), 2.04-1.81 (m, 2H), 1.30-1.19 (m, 2H), 1.07- |
| | | | | 0.90 (m, 1H) |
| K419 | GB | I | 859.4 | 11.06 (s, 1H), 8.92-8.87 (m, 1H), 8.68-8.62 (m, 2H), |
| | | | | 8.29 (dd, J = 8.4, 2.4 Hz, 1H), 8.04-7.96 (m, 2H), |
| | | | | 7.92 (s, 1H), 7.61 (dd, J = 7.1, 1.7 Hz, 1H), 7.54-7.47 |
| | | | | (m, 1H), 6.95 (d, J = 8.6 Hz, 1H), 6.86 (d, J = 2.2 Hz, |
| | | | | 1H), 6.65 (dd, J = 8.6, 2.2 Hz, 1H), 6.45 (t, J = 7.2 |
| | | | | Hz, 1H), 6.40 (s, 1H), 5.30 (dd, J = 12.9, 5.4 Hz, |
| | | | | 1H), 4.99-4.77 (m, 1H), 4.52-4.43 (m, 1H), 4.39-4.30 |
| | | | | (m, 1H), 4.22-4.14 (m, 1H), 4.04-3.97 (m, 1H), 3.32 |
| | | | | (s, 3H), 3.29-3.24 (m, 1H), 3.19-3.09 (m, 4H), 3.04- |
| | | | | 2.96 (m, 1H), 2.94-2.84 (m, 4H), 2.75-2.63 (m, 2H), |
| | | | | 2.62-2.52 (m, 4H), 2.03-1.96 (m, 1H), 1.28-1.21 (m, |
| | | | | 1H), 1.07-0.92 (m, 1H) |
| K420 | GJ | I | 898.4 | 11.09 (s, 1H), 8.88 (d, J = 2.3 Hz, 1H), 8.67-8.61 (m, |
| | | | | 2H), 8.26 (dd, J = 8.5, 2.3 Hz, 1H), 8.04-7.95 (m, |
| | | | | 2H), 7.92 (s, 1H), 7.59 (dd, J = 7.1, 1.7 Hz, 1H), |
| | | | | 7.54-7.46 (m, 1H), 7.24 (s, 1H), 7.12-7.07 (m, 2H), |
| | | | | 6.44 (t, J = 7.2 Hz, 1H), 6.40 (s, 1H), 5.41-5.31 (m, |
| | | | | 1H), 5.00-4.76 (m, 1H), 4.38-4.25 (m, 2H), 4.08-3.90 |
| | | | | (m, 2H), 3.72-3.59 (m, 2H), 3.34 (s, 3H), 3.05-2.96 |
| | | | | (m, 1H), 2.93-2.83 (m, 4H), 2.74-2.59 (m, 7H), 2.49- |
| | | | | 2.42 (m, 1H), 2.05-1.98 (m, 1H), 1.30-1.21 (m, 3H), |
| | | | | 1.07-0.92 (m, 1H) |
| K421 | ED | I | 857.4 | 11.12 (s, 1H), 8.70-8.63 (m, 3H), 8.10 (dd, J = 8.3, |
| | | | | 2.3 Hz, 1H), 8.00 (dd, J = 7.4, 1.8 Hz, 1H), 7.97-7.91 |
| | | | | (m, 2H), 7.62-7.58 (m, 1H), 7.54-7.47 (m, 1H), 7.21- |
| | | | | 7.12 (m, 2H), 7.08-7.00 (m, 1H), 6.46-6.39 (m, 2H), |
| | | | | 5.41 (dd, J = 12.7, 5.4 Hz, 1H), 4.99-4.76 (m, 1H), |
| | | | | 4.55 (s, 2H), 4.10-3.96 (m, 1H), 3.94-3.84 (m, 1H), |
| | | | | 3.66 (s, 3H), 3.61-3.52 (m, 1H), 3.45-3.40 (m, 1H), |
| | | | | 3.04-2.96 (m, 1H), 2.95-2.83 (m, 5H), 2.78-2.69 (m, |
| | | | | 1H), 2.67-2.59 (m, 1H), 2.06-1.89 (m, 3H), 1.68-1.52 |
| | | | | (m, 2H), 1.31-1.21 (m, 1H), 1.06-0.93 (m, 1H) |
| K422 | HE | I | 992.5 | 11.11 (s, 1H), 8.71-8.60 (m, 3H), 8.42 (d, J = 2.6 Hz, |
| | | | | 1H), 8.09-7.98 (m, 2H), 7.93 (d, J = 9.0 Hz, 2H), |
| | | | | 7.82 (dd, J = 8.9, 2.6 Hz, 1H), 7.58 (dd, J = 7.1, 1.7 |
| | | | | Hz, 1H), 7.54-7.39 (m, 1H), 7.43 (d, J = 1.8 Hz, 1H), |
| | | | | 7.26 (dd, J = 8.2, 1.7 Hz, 1H), 7.14 (d, J = 8.2 Hz, |
| | | | | 1H), 6.71 (d, J = 8.9 Hz, 1H), 6.46-6.38 (m, 2H), |
| | | | | 5.39 (dd, J = 12.8, 5.4 Hz, 1H), 4.98-4.77 (m, 1H), |
| | | | | 4.50-4.45 (m, 1H), 3.75 (s, 2H), 3.66 (t, J = 5.9 Hz, |
| | | | | 2H), 3.62-3.58 (m, 1H), 3.41 (s, 3H), 3.14-3.11 (m, |
| | | | | 1H), 3.02-2.97 (m, 1H), 2.93-2.89 (m, 1H), 2.88 (d, J = |
| | | | | 4.9 Hz, 3H), 2.84-2.76 (m, 4H), 2.69-2.55 (m, 4H), |
| | | | | 2.35-2.31 (m, 2H), 2.08-2.00 (m, 1H), 1.90-1.84 (m, |
| | | | | 2H), 1.82-1.74 (m, 2H), 1.70-1.66 (m, 1H), 1.29-1.19 |
| | | | | (m, 1H), 1.17-1.05 (m, 1H), 1.05-0.92 (m, 1H) |
| K423 | HG | I | 1027.5 | 11.08 (s, 1H), 8.78-8.44 (m, 3H), 8.11 (dd, J = 8.3, |
| | | | | 2.3 Hz, 1H), 8.04-7.94 (m, 2H), 7.92 (s, 1H), 7.59 |
| | | | | (dd, J = 7.1, 1.7 Hz, 1H), 7.54-7.50 (m, 1H), 7.06- |
| | | | | 6.97 (m, 2H), 6.87 (dd, J = 8.1, 1.6 Hz, 1H), 6.48- |
| | | | | 6.37 (m, 2H), 5.34 (dd, J = 12.7, 5.4 Hz, 1H), 4.99- |
| | | | | 4.79 (m, 1H), 3.67-3.53 (m, 8H), 3.41-3.37 (m, 2H), |
| | | | | 3.33 (s, 3H), 3.27-3.20 (m, 2H), 3.05-2.99 (m, 1H), |
| | | | | 2.93-2.87 (m, 4H), 2.77-2.64 (m, 4H), 2.61-2.53 (m, |
| | | | | 4H), 2.23-2.17 (m, 2H), 2.09-1.75 (m, 6H), 1.44-1.35 |
| | | | | (m, 2H), 1.33-1.19 (m, 2H), 1.05-0.95 (m, 1H), 0.87- |
| | | | | 0.82 (m, 1H) |
| K424 | DF | I | 872.3 | 11.04 (s, 1H), 8.70-8.63 (m, 3H), 8.11-7.92 (m, 4H), 7.62- |
| | | | | 7.46 (m, 2H), 6.90 (d, J = 8.4 Hz, 1H), 6.47-6.38 (m, 3H), |
| | | | | 6.22 (d, J = 8.6 Hz, 1H), 5.35-5.22 (m, 1H), 4.97-4.80 (m, |
| | | | | 1H), 4.62-4.49 (m, 1H), 3.73-3.58 (m, 1H), 3.46-3.38 (m, |
| | | | | 2H), 3.30 (s, 3H), 3.27-3.08 (m, 4H), 3.03-2.94 (m, 2H), 2.88 |
| | | | | (d, J = 4.8 Hz, 3H), 2.71-2.58 (m, 2H), 2.17-2.06 (m, 2H), |
| | | | | 2.04-1.94 (m, 2H), 1.92-1.83 (m, 1H), 1.77-1.57 (m, 2H), |
| | | | | 1.30-1.26 (m, 3H), 1.07-0.92 (m, 1H) |
| K425 | DG | I | 872.4 | 11.04 (s, 1H), 8.73-8.57 (m, 3H), 8.09 (d, J = 8.4 Hz, 1H), |
| | | | | 8.04-7.90 (m, 3H), 7.61 (d, J = 7.1 Hz, 1H), 7.53-7.47 (m, |
| | | | | 1H), 6.90 (d, J = 8.5 Hz, 1H), 6.47-6.33 (m, 3H), 6.22 (d, J = |
| | | | | 8.6 Hz, 1H), 5.36-5.22 (m, 1H), 5.00-4.77 (m, 1H), 4.63-4.47 |
| | | | | (m, 1H), 3.68-3.62 (m, 1H), 3.55-3.48 (m, 2H), 3.30 (s, 3H), |
| | | | | 3.25-3.10 (m, 4H), 3.04-2.93 (m, 2H), 2.88 (d, J = 4.8 Hz, |
| | | | | 3H), 2.71-2.57 (m, 2H), 2.19-2.06 (m, 2H), 2.04-1.92 (m, |
| | | | | 2H), 1.90-1.82 (m, 1H), 1.79-1.54 (m, 2H), 1.39-1.26 (m, |
| | | | | 3H), 1.06-0.93 (m, 1H) |
| K427 | GT | I | 915.5 | 11.07 (s, 1H), 8.69-8.65 (m, 3H), 8.10-8.05 (m, 1H), |
| | | | | 8.01 (d, J = 7.2 Hz, 1H), 7.98-7.95 (m, 1H), 7.92 (s, |
| | | | | 1H), 7.65-7.58 (m, 1H), 7.54-7.50 (m, 1H), 6.96 (d, J = |
| | | | | 8.5 Hz, 1H), 6.86 (s, 1H), 6.66 (d, J = 8.6 Hz, 1H), |
| | | | | 6.44 (t, J = 7.2 Hz, 1H), 6.40 (s, 1H), 5.30 (dd, J = |
| | | | | 12.8, 5.3 Hz, 1H), 4.98-4.78 (m, 1H), 3.17-3.12 (m, |
| | | | | 4H), 3.03-2.98 (m, 1H), 2.95-2.85 (m, 8H), 2.75-2.53 |
| | | | | (m, 8H), 2.30-1.91 (m, 7H), 1.50-1.43(m, 3H), 1.29- |
| | | | | 1.20 (m, 2H), 1.05-0.95 (m, 1H) |
| K428 | GR | I | 957.4 | 11.09 (s, 1H), 9.01 (d, J = 2.3 Hz, 1H), 8.68 (s, 1H), |
| | | | | 8.65 (d, J = 4.2 Hz, 1H), 8.55 (d, J = 7.8 Hz, 1H), |
| | | | | 8.39 (dd, J = 8.4, 2.3 Hz, 1H), 8.01 (dd, J = 7.4, 1.8 |
| | | | | Hz, 1H), 7.97 (d, J = 8.5 Hz, 1H), 7.92 (s, 1H), 7.58 |
| | | | | (dd, J = 7.2, 1.7 Hz, 1H), 7.54-7.50 (m, 1H), 7.10 (d, |
| | | | | J = 1.4 Hz, 1H), 7.05 (d, J = 8.0 Hz, 1H), 6.96 (dd, J = |
| | | | | 8.2, 1.5 Hz, 1H), 6.44 (t, J = 7.2 Hz, 1H), 6.41 (s, |
| | | | | 1H), 5.36 (dd, J = 12.7, 5.4 Hz, 1H), 4.98-4.78 (m, |
| | | | | 1H), 3.81-3.73 (m, 1H), 3.46 (s, 2H), 3.35 (s, 3H), |
| | | | | 3.03-2.97 (m, 1H), 2.93-2.84 (m, 6H), 2.78-2.59 (m, |
| | | | | 2H), 2.35-2.10 (m, 7H), 2.05-1.98 (m, 1H), 1.94-1.80 |
| | | | | (m, 6H), 1.65-1.60 (m, 2H), 1.52-1.20 (m, 7H), 1.01- |
| | | | | 0.85 (m, 1H) |
| K429 | HI | I | 887.5 | 11.08 (s, 1H), 8.88 (d, J = 2.3 Hz, 1H), 8.71-8.61 (m, |
| | | | | 2H), 8.32-8.24 (m, 1H), 8.02-7.97 (m, 2H), 7.92 (s, |
| | | | | 1H), 7.60 (dd, J = 7.2, 1.7 Hz, 1H), 7.53-7.49 (m, |
| | | | | 1H), 7.07 (s, 1H), 7.01 (d, J = 8.0 Hz, 1H), 6.90 (d, J = |
| | | | | 8.1 Hz, 1H), 6.44 (t, J = 7.3 Hz, 1H), 6.40 (s, 1H), |
| | | | | 5.34 (dd, J = 12.7, 5.4 Hz, 1H), 4.98-4.78 (m, 1H), |
| | | | | 4.44-4.39 (m, 1H), 4.31-4.26 (m, 1H), 4.16-4.10 (m, |
| | | | | 1H), 3.97-3.91 (m, 1H), 3.23-3.19 (m, 1H), 3.02-2.98 |
| | | | | (m, 1H), 2.96-2.83 (m, 4H), 2.78-74 (m, 2H), 2.73- |
| | | | | 2.59 (m, 2H), 2.55-2.51 (m, 8H), 2.40-2.29 (m, 5H), |
| | | | | 2.03-1.98 (m, 1H), 1.28-1.21 (m, 1H), 1.07-0.93 (m, |
| | | | | 1H) |
| K430 | HO | I | 969.5 | 11.10 (s, 1H), 9.01 (d, J = 2.4 Hz, 1H), 8.68 (s, 1H), |
| | | | | 8.65 (d, J = 4.2 Hz, 1H), 8.58 (d, J = 7.8 Hz, 1H), |
| | | | | 8.39 (dd, J = 8.5, 2.4 Hz, 1H), 8.01 (dd, J = 7.3, 1.8 |
| | | | | Hz, 1H), 7.97 (d, J = 8.5 Hz, 1H), 7.92 (s, 1H), 7.58 |
| | | | | (dd, J = 7.0, 1.7 Hz, 1H), 7.54-7.50 (m, 1H), 7.06 (d, |
| | | | | J = 6.9 Hz, 1H), 6.98-6.94 (m, 2H), 6.44 (t, J = 7.2 |
| | | | | Hz, 1H), 6.41 (s, 1H), 5.38 (dd, J = 12.4, 5.4 Hz, |
| | | | | 1H), 4.98-4.78 (m, 1H), 3.84-3.80 (m, 2H), 3.66 (s, |
| | | | | 3H), 3.09-2.90 (m, 8H), 2.87 (d, J = 4.8 Hz, 3H), |
| | | | | 2.78-2.60 (m, 2H), 2.50-2.45 (m, 2H), 2.36-2.08 (m, |
| | | | | 2H), 2.07-1.58 (m, 9H), 1.56-1.16 (m, 5H), 1.10-0.75 |
| | | | | (m, 3H) |
| K434 | FF | I | 884.4 | 11.13 (s, 1H), 8.74 (d, J = 2.3 Hz, 1H), 8.68-8.62 (m, |
| | | | | 2H), 8.15 (dd, J = 8.2, 2.3 Hz, 1H), 8.05-7.97 (m, |
| | | | | 2H), 7.92 (s, 1H), 7.60 (d, J = 7.0 Hz, 1H), 7.56-7.49 |
| | | | | (m, 1H), 7.41-7.30 (m, 1H), 7.27-7.14 (m, 2H), 6.44 |
| | | | | (t, J = 7.2 Hz, 1H), 6.40 (s, 1H), 5.40 (dd, J = 12.7, |
| | | | | 5.4 Hz, 1H), 4.98-4.76 (m, 1H), 4.01-3.86 (m, 2H), |
| | | | | 3.81-3.57 (m, 4H), 3.54-3.42 (m, 2H), 3.35 (s, 3H), |
| | | | | 3.33-3.29 (m, 4H), 3.04-2.96 (m, 1H), 2.94-2.83 (m, |
| | | | | 4H), 2.77-2.59 (m, 2H), 2.07-2.00 (m, 1H), 1.31-1.19 |
| | | | | (m, 1H), 1.06-0.92 (m, 1H) |
| K439 | GD | I | 894.4 | 11.11 (s, 1H), 8.80-8.59 (m, 3H), 8.15 (d, J = 8.3 Hz, |
| | | | | 1H), 8.05-7.95 (m, 2H), 7.92 (s, 1H), 7.65-7.47 (m, |
| | | | | 4H), 7.43-7.37 (m, 1H), 7.30-7.24 (m, 1H), 7.13 (d, J = |
| | | | | 8.2 Hz, 1H), 6.70 (d, J = 8.1 Hz, 2H), 6.46-6.38 |
| | | | | (m, 2H), 5.38 (dd, J = 12.8, 5.4 Hz, 1H), 4.98-4.76 |
| | | | | (m, 1H), 3.60-3.45 (m, 3H), 3.40 (s, 3H), 3.37 (s, |
| | | | | 3H), 3.05-2.91 (m, 4H), 2.88 (d, J = 4.6 Hz, 3H), |
| | | | | 2.77-2.61 (m, 2H), 2.40-2.21 (m, 2H), 2.09-2.00 (m, |
| | | | | 1H), 1.27-1.20 (m, 1H), 1.05-0.93 (m, 1H) |
| K440 | GF | I | 924.5 | 11.12 (s, 1H), 8.71-8.62 (m, 3H), 8.09 (dd, J = 8.3, |
| | | | | 2.3 Hz, 1H), 8.04-7.90 (m, 3H), 7.60 (dd, J = 7.1, 1.7 |
| | | | | Hz, 1H), 7.55-7.47 (m, 2H), 7.41-7.32 (m, 2H), 7.30- |
| | | | | 7.22 (m, 2H), 7.19 (d, J = 8.2 Hz, 1H), 6.97-6.90 (m, |
| | | | | 1H), 6.48-6.38 (m, 2H), 5.41 (dd, J = 12.8, 5.4 Hz, |
| | | | | 1H), 4.99-4.75 (m, 1H), 4.21 (t, J = 5.6 Hz, 2H), |
| | | | | 3.75-3.67 (m, 1H), 3.47-3.44 (m, 1H), 3.42 (s, 3H), |
| | | | | 3.30-3.23 (m, 2H), 3.04-2.91 (m, 2H), 2.88 (d, J = |
| | | | | 4.8 Hz, 3H), 2.84-2.71 (m, 3H), 2.69-2.54 (m, 5H), |
| | | | | 2.10-2.01 (m, 1H), 1.32-1.19 (m, 1H), 1.06-0.93 (m, |
| | | | | 1H) |
| K441 | GH | I | 960.4 | 11.15 (s, 1H), 8.93 (d, J = 13.8 Hz, 2H), 8.83 (t, J = |
| | | | | 2.9 Hz, 1H), 8.70-8.61 (m, 2H), 8.27-8.19 (m, 1H), |
| | | | | 8.04-7.95 (m, 2H), 7.92 (d, J = 2.7 Hz, 1H), 7.64- |
| | | | | 7.55 (m, 1H), 7.54-7.44 (m, 2H), 7.37-7.28 (m, 1H), |
| | | | | 7.22-7.19 (m, 1H), 6.49-6.38 (m, 2H), 5.47-5.37 (m, |
| | | | | 1H), 4.99-4.77 (m, 1H), 4.14-3.94 (m, 2H), 3.84- |
| | | | | 3.79 (m, 1H), 3.77-3.59 (m, 3H), 3.38 (s, 3H), 3.03- |
| | | | | 2.96 (m, 1H), 2.96-2.90 (m, 1H), 2.87 (d, J = 4.9 Hz, |
| | | | | 3H), 2.80-2.69 (m, 1H), 2.68-2.60 (m, 1H), 2.37- |
| | | | | 2.19 (m, 2H), 2.19-2.04 (m, 3H), 1.30-1.20 (m, 1H), |
| | | | | 1.05-0.92 (m, 1H) |
| K442 | BM | I | 873.4 | 11.10 (s, 1H), 8.74-8.61 (m, 3H), 8.09 (dd, J = 8.3, |
| | | | | 2.3 Hz, 1H), 8.04-7.92 (m, 3H), 7.63-7.58 (m, 1H), |
| | | | | 7.54-7.47 (m, 1H), 7.11-6.95 (m, 3H), 6.47-6.39 (m, |
| | | | | 2H), 5.37 (dd, J = 12.6, 5.4 Hz, 1H), 4.99-4.75 (m, |
| | | | | 1H), 4.20-4.11 (m, 1H), 4.10-3.96 (m, 2H), 3.68-3.60 |
| | | | | (m, 2H), 3.57 (s, 3H), 3.43-3.21 (m, 2H), 3.04-2.96 |
| | | | | (m, 1H), 2.94-2.83 (m, 4H), 2.77-2.57 (m, 3H), 2.47- |
| | | | | 2.39 (m, 1H), 2.15-2.03 (m, 2H), 2.03-1.79 (m, 3H), |
| | | | | 1.62-1.41 (m, 2H), 1.29-1.19 (m, 1H), 1.07-0.92 (m, |
| | | | | 1H) |
| K443 | CW | I | 920.3 | 11.11 (s, 1H), 8.87-8.79 (m, 1H), 8.73-8.59 (m, 2H), |
| | | | | 8.30-8.15 (m, 1H), 8.04-7.94 (m, 2H), 7.92 (d, J = |
| | | | | 4.1 Hz, 1H), 7.65-7.47 (m, 4H), 7.43-7.35 (m, 1H), |
| | | | | 7.25 (t, J = 9.4 Hz, 1H), 7.12 (t, J = 7.6 Hz, 1H), 6.62 |
| | | | | (dd, J = 23.6, 8.3 Hz, 2H), 6.48-6.36 (m, 2H), 5.42- |
| | | | | 5.33 (m, 1H), 4.99-4.75 (m, 1H), 3.75-3.65 (m, 2H), |
| | | | | 3.63-3.53 (m, 2H), 3.45-3.41 (m, 2H), 3.40 (d, J = |
| | | | | 6.0 Hz, 3H), 3.26-3.15 (m, 2H), 3.03-2.95 (m, 1H), |
| | | | | 2.93-2.83 (m, 4H), 2.80-2.58 (m, 2H), 2.21-1.89 (m, |
| | | | | 5H), 1.32-1.16 (m, 1H), 1.07-0.88 (m, 1H) |
| K444 | FY | I | 993.4 | 11.12 (s, 1H), 9.01 (d, J = 2.3 Hz, 1H), 8.69 (s, 1H), |
| | | | | 8.65 (d, J = 4.1 Hz, 1H), 8.59 (d, J = 7.5 Hz, 1H), |
| | | | | 8.39 (dd, J = 8.5, 2.4 Hz, 1H), 8.02 (d, J = 7.4 Hz, |
| | | | | 1H), 7.98 (dd, J = 8.4, 2.2 Hz, 1H), 7.92 (s, 1H), |
| | | | | 7.62-7.56 (m, 1H), 7.54-7.50 (m, 1H), 7.32 (d, J = |
| | | | | 1.4 Hz, 1H), 7.19-7.10 (m, 2H), 6.50-6.36 (m, 2H), |
| | | | | 5.46-5.32 (m, 1H), 4.98-4.78 (m, 1H), 3.80-3.73 (m, |
| | | | | 1H), 3.65 (s, 1H), 3.56-3.51 (m, 1H), 3.50-3.46 (m, |
| | | | | 2H), 3.43-3.38 (m, 1H), 3.38-3.32 (m, 4H), 3.32-3.28 |
| | | | | (m, 1H), 3.27-3.20 (m, 2H), 3.02-2.97 (m, 1H), 2.89- |
| | | | | 2.85 (m, 4H), 2.78-2.59 (m, 2H), 2.45-2.35 (m, 2H), |
| | | | | 2.14-1.99 (m, 2H), 1.97-1.92 (m, 3H), 1.82-1.75 (m, |
| | | | | 2H), 1.59-1.36 (m, 4H), 1.27-1.21 (m, 1H), 1.05-0.95 |
| | | | | (m, 1H) |
| K445 | GP | I | 915.5 | 11.09 (s, 1H), 8.82-8.76 (m, 1H), 8.67-8.64 (m, 2H), |
| | | | | 8.21-8.17 ( m, J = 8.0, 1H), 8.01 (dd, J = 7.4, 1.8 Hz, |
| | | | | 1H), 7.96 (dd, J = 8.4, 2.7 Hz, 1H), 7.92 (s, 1H), |
| | | | | 7.62-7.58 (m, 1H), 7.54-7.50 (m, 1H), 6.93 (dd, J = |
| | | | | 8.6, 5.5 Hz, 1H), 6.83 (dd, J = 10.3, 2.2 Hz, 1H), |
| | | | | 6.70-6.58 (m, 1H), 6.48-6.35 (m, 2H), 5.28 (dd, J = |
| | | | | 10.4, 5.7 Hz, 1H), 4.98-4.78 (m, 1H), 3.74-3.46 (m, |
| | | | | 5H), 3.32-3.29 (m, 5H), 3.27-3.24 (m, 1H), 3.03-2.97 |
| | | | | (m, 1H), 2.95-2.85 (m, 3H), 2.75-2.55 (m, 3H), 2.49- |
| | | | | 2.31 (m, 3H), 2.28-2.19 (m, 3H), 2.07-1.94 (m, 2H), |
| | | | | 1.83-1.76 (m, 1H), 1.74-1.57 (m, 4H), 1.57-1.45 (m, |
| | | | | 1H), 1.29-1.20 (m, 1H), 1.05-0.95 (m, 1H) |
| K446 | DE | I | 991.5 | 11.11 (s, 1H), 8.71-8.62 (m, 3H), 8.09 (dd, J = 8.3, |
| | | | | 2.3 Hz, 1H), 8.03-7.98 (m, 1H), 7.97-7.91 (m, 2H), |
| | | | | 7.59 (dd, J = 7.2, 1.7 Hz, 1H), 7.55-7.48 (m, 3H), |
| | | | | 7.40-7.35 (m, 1H), 7.27-7.20 (m, 1H), 7.11 (d, J = |
| | | | | 8.3 Hz, 1H), 6.60 (d, J = 8.4 Hz, 2H), 6.47-6.38 (m, |
| | | | | 2H), 5.42-5.30 (m, 1H), 5.01-4.77 (m, 1H), 4.64-4.50 |
| | | | | (m, 1H), 3.70-3.59 (m, 1H), 3.39 (s, 3H), 3.30-3.23 |
| | | | | (m, 2H), 3.19-3.09 (m, 1H), 3.05-2.96 (m, 2H), 2.95- |
| | | | | 2.90 (m, 1H), 2.87 (d, J = 4.8 Hz, 3H), 2.80-2.60 (m, |
| | | | | 4H), 2.58-2.53 (m, 1H), 2.29 (s, 3H), 2.14-1.93 (m, |
| | | | | 3H), 1.88-1.80 (m, 1H), 1.79-1.64 (m, 2H), 1.57-1.40 |
| | | | | (m, 2H), 1.28-1.22 (m, 3H), 1.06-0.91 (m, 1H) |
| K447 | CX | I | 971.4 | 11.08 (s, 1H), 9.03-8.97 (m, 1H), 8.72-8.61 (m, 2H), |
| | | | | 8.44-8.35 (m, 1H), 8.18 (d, J = 8.7 Hz, 1H), 8.06- |
| | | | | 7.95 (m, 2H), 7.92 (s, 1H), 7.58 (d, J = 7.0 Hz, 1H), |
| | | | | 7.53-7.46 (m, 1H), 7.13-7.07 (m, 1H), 7.07-7.02 (m, |
| | | | | 1H), 7.01-6.94 (m, 1H), 6.48-6.37 (m, 2H), 5.36 (dd, |
| | | | | J = 12.7, 5.4 Hz, 1H), 5.00-4.73 (m, 1H), 4.00-3.71 |
| | | | | (m, 2H), 3.50 (s, 2H), 3.04-3.00 (m, 1H), 2.91-2.79 |
| | | | | (m, 5H), 2.77-2.60 (m, 3H), 2.85-2.78 (m, 2H), 2.76- |
| | | | | 2.56 (m, 2H), 2.22-2.05 (m, 6H), 2.04-1.82 (m, 5H), |
| | | | | 1.74-1.57 (m, 3H), 1.55-1.20 (m, 5H), 1.15-0.89 (m, |
| | | | | 5H) |
| K448 | HK | I | 998.5 | 11.09 (s, 1H), 9.05-8.99 (m, 1H), 8.68 (s, 1H), 8.65 |
| | | | | (d, J = 4.2 Hz, 1H), 8.56 (d, J = 7.6 Hz, 1H), 8.45- |
| | | | | 8.36 (m, 1H), 8.01 (dd, J = 7.4, 1.7 Hz, 1H), 7.97 (d, |
| | | | | J = 8.4 Hz, 1H), 7.92 (d, J = 1.1 Hz, 1H), 7.58 (dd, J = |
| | | | | 7.1, 1.7 Hz, 1H), 7.53-7.49 (m, 1H), 7.10 (s, 1H), |
| | | | | 7.05 (d, J = 8.1 Hz, 1H), 6.97 (d, J = 8.2 Hz, 1H), |
| | | | | 6.44 (t, J = 7.2 Hz, 1H), 6.41 (s, 1H), 5.36 (dd, J = |
| | | | | 12.6, 5.4 Hz, 1H), 4.97-4.77 (m, 1H), 3.85-3.69 (m, |
| | | | | 2H), 3.47 (s, 2H), 3.34 (s, 3H), 3.04-2.98 (m, 1H), |
| | | | | 2.93-2.86 (m, 4H), 2.84-2.56 (m, 3H), 2.47-2.22 (m, |
| | | | | 12H), 2.08-1.97 (m, 1H), 1.94-1.79 (m, 4H), 1.68- |
| | | | | 1.50 (m, 3H), 1.44-1.19 (m, 4H), 1.05-0.89 (m, 3H) |
| K449 | HM | I | 957.5 | 11.10 (s, 1H), 9.01 (d, J = 2.3 Hz, 1H), 8.68 (s, 1H), |
| | | | | 8.65 (d, J = 4.2 Hz, 1H), 8.55 (d, J = 7.7 Hz, 1H), |
| | | | | 8.39 (dd, J = 8.5, 2.4 Hz, 1H), 8.01 (dd, J = 7.4, 1.8 |
| | | | | Hz, 1H), 7.97 (d, J = 8.4 Hz, 1H), 7.92 (s, 1H), 7.60- |
| | | | | 7.56 (m, 1H), 7.53-7.49 (m, 1H), 7.10-7.06 (m, 1H), |
| | | | | 6.96 (t, J = 7.7 Hz, 1H), 6.87 (d, J = 7.6 Hz, 1H), |
| | | | | 6.44 (t, J = 7.2 Hz, 1H), 6.40 (s, 1H), 5.39 (dd, J = |
| | | | | 12.6, 5.4 Hz, 1H), 4.98-4.78 (m, 1H), 3.83-3.72 (m, |
| | | | | 1H), 3.68 (s, 3H), 3.62 (s, 2H), 3.51-3.45 (m, 2H) |
| | | | | 3.03-2.98 (m, 1H), 2.95-2.84 (m, 4H), 2.82-2.60 (m, |
| | | | | 2H), 2.35-2.30 (m, 1H), 2.22-2.15 (m, 4H), 2.04-1.96 |
| | | | | (m, 4H), 1.86-1.79 (m, 2H), 1.66-1.61 (m, 2H), 1.43- |
| | | | | 1.21 (m, 7H), 1.08-0.79 (m, 4H) |
| K450 | HQ | I | 872.5 | 11.04 (s, 1H), 8.87 (d, J = 2.4 Hz, 1H), 8.68-8.64 (m, |
| | | | | 2H), 8.27 (dd, J = 8.4, 2.4 Hz, 1H), 8.02-7.98 (m, |
| | | | | 2H), 7.92 (s, 1H), 7.60 (dd, J = 7.1, 1.7 Hz, 1H), |
| | | | | 7.53-7.48 (m, 1H), 7.06-6.93 (m, 3H), 6.44 (t, J = 7.2 |
| | | | | Hz, 1H), 6.40 (s, 1H), 5.37 (dd, J = 12.6, 5.4 Hz, |
| | | | | 1H), 5.01-4.72 (m, 1H), 4.51 (t, J = 8.2 Hz, 1H), 4.20 |
| | | | | (t, J = 9.2 Hz, 1H), 4.12-4.08 (m, 1H), 3.80-3.76 (m, |
| | | | | 1H), 3.58 (s, 3H), 3.25-3.18 (m, 1H), 3.07-2.91 (m, |
| | | | | 3H), 2.88 (d, J = 4.8 Hz, 3H), 2.77-2.57 (m, 4H), |
| | | | | 2.17-2.09 (m, 2H), 2.02-1.96 (m, 1H), 1.81-1.74 (m, |
| | | | | 4H), 1.34-1.11 (m, 3H), 1.06-0.93 (m, 1H) |
| K451 | FM | I | 900.3 | 11.08 (s, 1H), 8.68-8.62 (m, 3H), 8.09-8.03 (m, 1H), |
| | | | | 8.01 (dd, J = 7.3, 1.8 Hz, 1H), 7.94 (d, J = 13.2 Hz, |
| | | | | 2H), 7.59 (dd, J = 7.1, 1.7 Hz, 1H), 7.56-7.48 (m, |
| | | | | 1H), 7.07-6.97 (m, 3H), 6.48-6.38 (m, 2H), 5.42-5.33 |
| | | | | (m, 1H), 5.01-4.76 (m, 1H), 4.53-4.46 (m, 1H), 3.65- |
| | | | | 3.61 (m, 1H), 3.58 (s, 3H), 3.47-3.39 (m, 1H), 3.17- |
| | | | | 3.13 (m, 1H), 3.04-2.95 (m, 2H), 2.88 (d, J = 4.9 Hz, |
| | | | | 3H), 2.85-2.81 (m, 3H), 2.78-2.67 (m, 1H), 2.67-2.58 |
| | | | | (m, 1H), 2.25-2.18 (m, 2H), 2.03-1.95 (m, 3H), 1.91- |
| | | | | 1.84 (m, 2H), 1.79-1.56 (m, 4H), 1.32-1.12 (m, 4H), |
| | | | | 1.07-0.92 (m, 1H) |
| K453 | CV | I | 970.5 | 11.06 (s, 1H), 8.71-8.62 (m, 3H), 8.12-8.06 (m, |
| | | | | 1H), 8.03-7.98 (m, 1H), 7.97-7.91 (m, 2H), 7.62-7.58 |
| | | | | (m, 1H), 7.55-7.49 (m, 1H), 6.97-6.90 (m, 1H), 6.85- |
| | | | | 6.81 (m, 1H), 6.65-6.59 (m, 1H), 6.46-6.43 (m, 1H), |
| | | | | 6.42-6.39 (m, 1H), 5.29 (dd, J = 12.6, 5.4 Hz, 1H), |
| | | | | 5.00-4.76 (m, 1H), 3.35 (s, 3H), 3.33-3.31(m, 4H), |
| | | | | 3.11-3.04 (m, 4H), 3.03-2.90 (m, 2H), 2.88 (d, J = |
| | | | | 4.9 Hz, 3H), 2.72-2.61 (m, 8H), 2.23-2.08 (m, 4H), |
| | | | | 2.04-1.93 (m, 1H), 1.92-1.60 (m, 4H), 1.54-1.32 (m, |
| | | | | 4H), 1.30-1.18 (m, 1H), 1.06-0.91 (m, 1H) |
| K462 | IG | I | 970.5 | 11.07 (s, 1H), 8.69-8.64 (m, 3H), 8.11-8.07 (m, 1H), |
| | | | | 8.03-7.98 (m, 1H), 7.95 (d, J = 8.2 Hz, 1H), 7.92 (s, |
| | | | | 1H), 7.60 (dd, J = 7.2, 1.7 Hz, 1H), 7.54-7.49 (m, |
| | | | | 1H), 7.01-6.96 (m, 1H), 6.94-6.84 (m, 2H), 6.44 (t, J = |
| | | | | 7.2 Hz, 1H), 6.41 (s, 1H), 5.35 (dd, J = 12.5, 5.4 |
| | | | | Hz, 1H), 4.99-4.79 (m, 1H), 4.56-4.48 (m, 1H), 3.66- |
| | | | | 3.58 (m, 5H), 3.21-2.78 (m, 14H), 2.77-2.54 (m, 2H), |
| | | | | 2.51-2.42 (m, 3H), 2.23-2.12 (m, 3H), 2.03-1.96 (m, |
| | | | | 1H), 1.90-1.69 (m, 4H), 1.53-1.36 (m, 4H), 1.29-1.20 |
| | | | | (m, 1H), 1.09-0.94 (m, 1H) |
| K616 | AC | JQ | 971.5 | 11.11 (s, 1H), 8.89 (d, J = 4.7 Hz, 1H), 8.74 (d, J = |
| | | | | 2.2 Hz, 1H), 7.98-7.94 (m, 2H), 7.88-7.79 (m, 2H), |
| | | | | 7.74-7.69 (m, 1H), 7.60 (d, J = 8.0 Hz, 2H), 7.47 (d, J = |
| | | | | 1.7 Hz, 1H), 7.40-7.28 (m, 5H), 7.18 (d, J = 8.3 Hz, |
| | | | | 1H), 6.03 (s, 1H), 5.40 (dd, J = 12.8, 5.4 Hz, 1H), |
| | | | | 4.99-4.77 (m, 1H), 4.17 (t, J = 8.5 Hz, 2H), 3.75-3.60 |
| | | | | (m, 2H), 3.48 (t, J = 8.5 Hz, 2H), 3.41 (s, 3H), 3.33- |
| | | | | 3.28 (m, 2H), 3.09-3.01 (m, 1H), 3.01-2.87 (m, 4H), |
| | | | | 2.81-2.57 (m, 6H), 2.47-2.41 (m, 2H), 2.09-2.00 (m, |
| | | | | 1H), 1.98-1.87 (m, 4H), 1.59-1.37 (m, 4H), 1.27-1.21 |
| | | | | (m, 1H), 1.04-0.91 (m, 1H) |
| K617 | AC | JA | 975.5 | 11.11 (s, 1H), 8.81 (d, J = 4.2 Hz, 1H), 8.77-8.73 (m, |
| | | | | 1H), 8.61 (s, 1H), 7.99-7.92 (m, 2H), 7.86 (s, 1H), |
| | | | | 7.78 (dd, J = 7.9, 1.7 Hz, 1H), 7.63-7.58 (m, 2H), |
| | | | | 7.54-7.45 (m, 3H), 7.34-7.29 (m, 3H), 7.28-7.24 (m, |
| | | | | 1H), 7.21-7.15 (m, 1H), 6.13 (s, 1H), 5.40 (dd, J = |
| | | | | 12.8, 5.4 Hz, 1H), 4.85-4.61 (m, 1H), 3.74-3.63 (m, |
| | | | | 2H), 3.54 (s, 3H), 3.41 (s, 3H), 3.32-3.31 (m, 4H), |
| | | | | 2.91 (d, J = 4.9 Hz, 3H), 2.89-2.82 (m, 1H), 2.81- |
| | | | | 2.71 (m, 1H), 2.70-2.56 (m, 5H), 2.48-2.41 (m, 1H), |
| | | | | 2.10-2.01 (m, 1H), 1.98-1.86 (m, 4H), 1.60-1.35 (m, |
| | | | | 4H), 1.12-1.01 (m, 1H), 0.71-0.59 (m, 1H) |
| K627 | JR | JQ | 986.5 | 11.11 (s, 1H), 8.88 (d, J = 4.7 Hz, 1H), 8.72 (d, J = |
| | | | | 2.2 Hz, 1H), 7.99-7.90 (m, 2H), 7.86-7.78 (m, 2H), |
| | | | | 7.74-7.69 (m, 1H), 7.55 (d, J = 8.5 Hz, 2H), 7.42 (d, J = |
| | | | | 1.7 Hz, 1H), 7.39-7.24 (m, 3H), 7.14 (d, J = 8.2 Hz, |
| | | | | 1H), 7.01 (d, J = 8.6 Hz, 2H), 6.01(s, 1H), 5.44-5.30 |
| | | | | (m, 1H), 4.97-4.77 (m, 1H), 4.56-4.48 (m, 1H), 4.17 |
| | | | | (t, J = 8.5 Hz, 2H), 3.49 (q, J = 8.5 Hz, 2H), 3.40 (s, |
| | | | | 3H), 3.23-3.12 (m, 4H), 3.09-3.03 (m, 1H), 3.01-2.84 |
| | | | | (m, 4H), 2.82-2.58 (m, 2H), 3.77-3.53 (m, 7H), 2.28- |
| | | | | 2.22 (m, 2H), 2.10-1.99 (m, 1H), 1.92-1.71 (m, 3H), |
| | | | | 1.26-1.17 (m, 3H), 1.08-0.81 (m, 1H) |
| K629 | JR | JA | 990.5 | 11.11 (s, 1H), 8.81 (d, J = 4.2 Hz, 1H), 8.73 (d, J = |
| | | | | 1.7 Hz, 1H), 8.61 (s, 1H), 7.92 (d, J = 1.6 Hz, 2H), |
| | | | | 7.87 (d, J = 1.7 Hz, 1H), 7.78 (dd, J = 7.9, 1.9 Hz, |
| | | | | 1H), 7.59-7.45 (m, 4H), 7.42 (d, J = 1.6 Hz, 1H), |
| | | | | 7.31-7.22 (m, 2H), 7.14 (d, J = 8.2 Hz, 1H), 7.01 (d, J = |
| | | | | 8.4 Hz, 2H), 6.13 (d, J = 1.7 Hz, 1H), 5.39 (dd, J = |
| | | | | 12.8, 5.4 Hz, 1H), 4.83-4.63 (m, 1H), 4.56-4.48 (m, |
| | | | | 1H), 3.45-3.30 (m, 5H), 3.22-3.13 (m, 4H), 2.98-2.81 |
| | | | | (m, 6H), 2.80-2.62 (m, 2H), 2.56-2.51 (m, 3H), 2.28- |
| | | | | 2.22 (m, 2H), 2.08-2.01 (m, 1H), 1.93-1.70 (m, 7H), |
| | | | | 1.20-1.00 (m, 3H), 0.70-0.60 (m, 1H) |
| K632 | AC | IX | 945.5 | 11.11 (s, 1H), 9.31 (s, 1H), 8.80 (d, J = 4.0 Hz, 1H), |
| | | | | 8.71 (d, J = 2.1 Hz, 1H), 8.24 (s, 1H), 8.07 (d, J = 8.2 |
| | | | | Hz, 1H), 7.94 (dd, J = 8.2, 2.2 Hz, 1H), 7.89 (s, 1H), |
| | | | | 7.78 (d, J = 7.7 Hz, 1H), 7.62-7.58 (m, 3H), 7.56- |
| | | | | 7.46 (m, 3H), 7.36-7.25 (m, 3H), 7.18 (d, J = 8.2 Hz, |
| | | | | 1H), 5.83 (s, 1H), 5.40 (dd, J = 12.8, 5.4 Hz, 1H), |
| | | | | 4.73-4.53 (m, 1H), 3.67 (s, 2H), 3.44-3.40 (m, 4H), |
| | | | | 3.34-3.27 (m, 2H), 2.93-2.89 (m, 4H), 2.83-2.56 (m, |
| | | | | 6H), 2.49-2.43 (m, 6H), 2.12-2.02 (m, 1H), 1.94-1.87 |
| | | | | (m, 4H), 1.57-1.36 (m, 4H), 0.98-0.83 (m, 1H), 0.67- |
| | | | | 0.57 (m, 1H) |
| K633 | AI | IX | 863.3 | 11.11 (s, 1H), 9.30 (s, 1H), 8.80 (d, J = 4.0 Hz, 1H), |
| | | | | 8.77 (d, J = 2.1 Hz, 1H), 8.26 (s, 1H), 8.10 (d, J = 8.2 |
| | | | | Hz, 1H), 8.00 (dd, J = 8.2, 2.2 Hz, 1H), 7.89 (s, 1H), |
| | | | | 7.80 (d, J = 7.5 Hz, 1H), 7.63-7.57 (m, 3H), 7.56- |
| | | | | 7.45 (m, 2H), 7.44 (d, J = 1.6 Hz, 1H), 7.32-7.28 (m, |
| | | | | 1H), 7.15 (d, J = 8.3 Hz, 1H), 7.09-7.05 (m, 2H), |
| | | | | 5.83 (s, 1H), 5.39 (dd, J = 12.8, 5.4 Hz, 1H), 4.78- |
| | | | | 4.44 (m, 1H), 3.89-3.50 (m, 4H), 3.42-3.40 (m, 4H), |
| | | | | 3.32-3.27 (m, 3H), 2.97-2.88 (m, 4H), 2.80-2.57 (m, |
| | | | | 3H), 2.08-2.02 (m, 1H), 0.96-0.78 (m, 1H), 0.69-0.52 |
| | | | | (m, 1H) |

^{a}The reductive amination was run from 25-50° C. under standard conditions. KOAc was added to free base amines when necessary. DMSO/DCE was also employed as a solvent mixture. Purification was run under a variety of typical methods including prep-HPLC, reverse phase chromatography, and silica gel chromatography.

### Example 2.2. Method 2: Synthesis of 6-{[5-({4-[1-(2,6-dioxopiperidin-3-yl)-3-methyl-2-oxo-1,3-benzodiazol-5-yl]piperazin-1-yl}methyl)pyridin-2-yl]amino}-4-{[3-(5-fluoropyrimidin-2-yl)-2-methoxyphenyl]amino}-N-methylpyridine-3-carboxamide

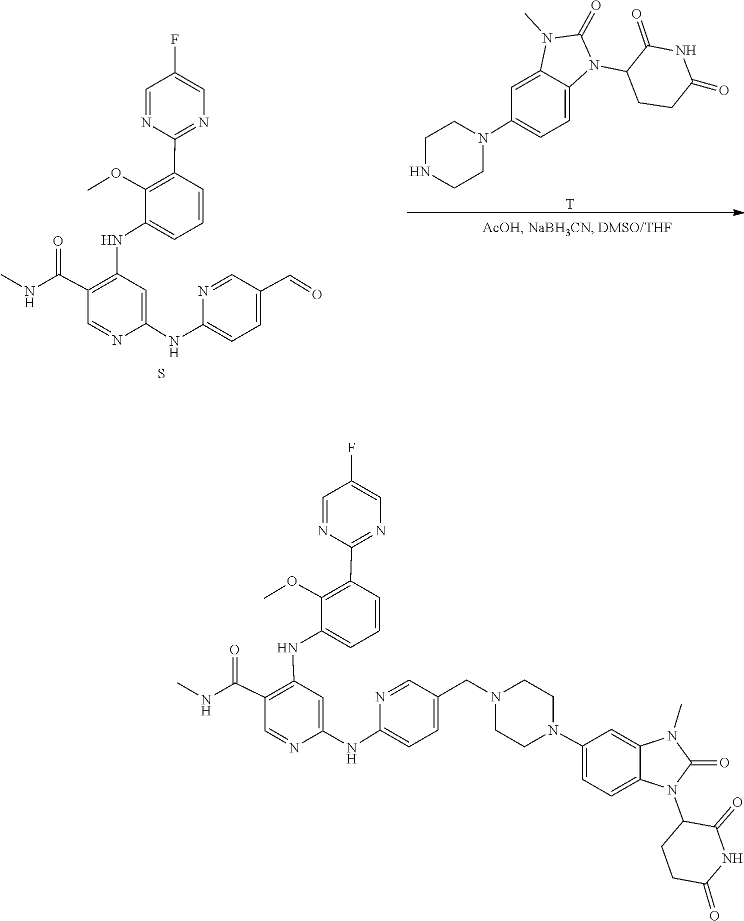

To a stirred mixture of 4-{[3-(5-fluoropyrimidin-2-yl)-2-methoxyphenyl]amino}-6-[(5-formylpyridin-2-yl)amino]-N-methylpyridine-3-carboxamide (100 mg, 0.211 mmol, Intermediate S) and 3-[3-methyl-2-oxo-5-(piperazin-1-yl)-1,3-benzodiazol-1-yl]piperidine-2,6-dione (72.53 mg, 0.211 mmol, Intermediate T) in DMSO (2 mL) and THF (2 mL) were added AcOH (12.68 mg, 0.211 mmol) and NaBH₃CN (66.36 mg, 1.055 mmol) in turns at rt. The resulting mixture was stirred for 16 h at 50° C. After completion, the reaction mixture was concentrated under vacuum. The residue was purified by reverse phase flash chromatography (Column: WelFlash™ C18-I, 20-40 um, 80 g; Eluent A: Water (plus 10 mmol/L NH₄HCO₃); Eluent B: ACN; Gradient: 25%-55% B in 25 min; Flow rate: 50 mL/min; Detector: 220/254 nm; desired fractions were collected at 50% B) and concentrated under reduced pressure to afford the title compound (12.5 mg, 7% yield) as a white solid. ¹H NMR (400 MHz, DMSO-d₆) δ 11.05 (s, 1H), 10.71 (s, 1H), 9.76 (s, 1H), 9.04 (s, 2H), 8.52-8.48 (m, 2H), 8.17-8.14 (m, 1H), 7.97 (s, 1H), 7.74 (dd, J=8.0, 1.7 Hz, 1H), 7.63 (dd, J=8.6, 2.3 Hz, 1H), 7.56 (d, J=8.5 Hz, 1H), 7.42 (dd, J=7.8, 1.6 Hz, 1H), 7.33 (t, J=7.9 Hz, 1H), 6.94 (d, J=8.6 Hz, 1H), 6.83 (d, J=2.2 Hz, 1H), 6.62 (dd, J=8.7, 2.2 Hz, 1H), 5.29 (dd, J=12.8, 5.4 Hz, 1H), 3.71 (s, 3H), 3.48 (s, 2H), 3.30 (s, 3H), 3.12-3.08 (s, 4H), 2.93-2.83 (m, 1H), 2.80 (d, J=4.4 Hz, 3H), 2.74-2.58 (3, 2H), 2.56-2.52 (6, 4H), 2.04-1.97 (m, 1H). LC/MS (ESI, m/z): [(M+H)]⁺=801.5.

TABLE D
Compounds synthesized via Method 2, via the reductive
amination of the corresponding amines and aldehyde.

| | | | LCMS | |
|---|---|---|---|---|
| | | | (ESI+) | |
| Compound | | | m/z | |
| ID (K#) | Amine | Aldehyde | (M + H)⁺ | ¹H NMR (400 MHz, DMSO-d6) |
| K132 | T | BX | 801.5 | 11.07 (s, 1H), 10.71 (s, 1H), 9.74 (s, 1H), 9.09- |
| | | | | 8.99 (m, 2H), 8.56-8.44 (m, 2H), 8.13 (d, J = 5.1 |
| | | | | Hz, 1H), 8.01 (s, 1H), 7.76-7.70 (m, 1H), 7.57 |
| | | | | (s, 1H), 7.41 (dd, J = 7.7, 1.6 Hz, 1H), 7.32 (t, J = |
| | | | | 7.9 Hz, 1H), 6.95 (d, J = 8.6 Hz, 1H), 6.90- |
| | | | | 6.80 (m, 2H), 6.64 (dd, J = 8.7, 2.2 Hz, 1H), |
| | | | | 5.30 (dd, J = 12.9, 5.3 Hz, 1H), 3.71 (s, 3H), |
| | | | | 3.52 (s, 2H), 3.31 (s, 3H), 3.1-3.07 (m, 4H), |
| | | | | 2.97-2.84 (m, 1H), 2.79 (d, J = 4.4 Hz, 3H), |
| | | | | 2.74-2.53 (m, 6H), 2.04-1.94 (m, 1H) |
| K176 | BW | BX | 801.4 | 11.08 (s, 1H), 10.71 (s, 1H), 9.72 (s, 1H), 9.05- |
| | | | | 9.03 (m, 2H), 8.55-8.47 (m, 2H), 8.14 (d, J = 5.1 |
| | | | | Hz, 1H), 8.01 (s, 1H), 7.74 (dd, J = 8.0, 1.7 Hz, |
| | | | | 1H), 7.56 (s, 1H), 7.41 (dd, J = 7.8, 1.6 Hz, 1H), |
| | | | | 7.32 (t, J = 7.9 Hz, 1H), 7.03-6.93 (m, 2H), |
| | | | | 6.92-6.86 (m, 2H), 5.35 (dd, J = 12.7, 5.3 Hz, |
| | | | | 1H), 3.71 (s, 3H), 3.64 (s, 3H), 3.53 (s, 2H), |
| | | | | 3.34-3.32 (m, 4H), 3.06-2.84 (m, 5H), 2.80 (d, J = |
| | | | | 4.4 Hz, 3H), 2.75-2.58 (m, 2H), 2.06-1.94 (m, 1H) |
| K175 | ES | S | 829.4 | 11.04 (s, 1H), 10.70 (s, 1H), 9.73 (s, 1H), 9.07- |
| | | | | 9.02 (m, 2H), 8.53-8.46 (m, 2H), 8.08-8.05 (m, |
| | | | | 1H), 7.95 (s, 1H), 7.73 (dd, J = 8.0, 1.7 Hz, 1H), |
| | | | | 7.61-7.51 (m, 2H), 7.44-7.39 (m, 1H), 7.33 (t, J = |
| | | | | 7.9 Hz, 1H), 6.89 (d, J = 8.6 Hz, 1H), 6.71- |
| | | | | 6.67 (m, 1H), 6.53 (dd, J = 8.7, 2.3 Hz, 1H), |
| | | | | 5.27 (dd, J = 12.8, 5.4 Hz, 1H), 3.70 (s, 3H), |
| | | | | 3.41 (s, 2H), 3.30 (s, 3H), 2.94-2.83 (m, 3H), |
| | | | | 2.80 (d, J = 4.4 Hz, 3H), 2.75-2.57 (m, 6H), |
| | | | | 2.10-1.94 (m, 3H), 1.75-1.54 (m, 4H) |
| K174 | DW | CY | 800.3 | 11.09 (s, 1H), 10.70 (s, 1H), 9.64 (s, 1H), 9.07- |
| | | | | 9.00 (m, 2H), 8.53-8.42 (m, 2H), 8.09-8.02 (m, |
| | | | | 1H), 7.94 (s, 1H), 7.72 (d, J = 7.9 Hz, 1H), 7.61- |
| | | | | 7.54 (m, 1H), 7.51-7.45 (m, 1H), 7.43-7.37 (m, |
| | | | | 1H), 7.37-7.30 (m, 1H), 7.12-7.04 (m, 1H), |
| | | | | 7.01-6.94 (m, 1H), 6.94-6.88 (m, 1H), 5.39 (dd, |
| | | | | J = 12.5, 5.5 Hz, 1H), 3.72 (s, 2H), 3.70 (s, 3H), |
| | | | | 3.34 (s, 3H), 2.99-2.84 (m, 3H), 2.79 (d, J = 4.4 |
| | | | | Hz, 3H), 2.76-2.58 (m, 2H), 2.15-1.94 (m, 4H), |
| | | | | 1.77-1.66 (m, 2H), 1.66-1.53 (m, 2H) |
| K173 | BW | S | 801.3 | 11.08 (s, 1H), 10.70 (s, 1H), 9.75 (s, 1H), 9.06- |
| | | | | 9.03 (m, 2H), 8.53-8.47 (m, 2H), 8.13-8.10 (m, |
| | | | | 1H), 7.97 (s, 1H), 7.74 (dd, J = 7.9, 1.6 Hz, 1H), |
| | | | | 7.65-7.60 (m, 1H), 7.59-7.53 (m, 1H), 7.45-7.40 |
| | | | | (m, 1H), 7.37-7.31 (m, 1H), 7.02-6.85 (m, 3H), |
| | | | | 5.35 (dd, J = 12.6, 5.4 Hz, 1H), 3.70 (s, 3H), |
| | | | | 3.63 (s, 3H), 3.49 (s, 2H), 3.31-3.28 (m, 4H), |
| | | | | 3.07-2.83 (m, 5H), 2.80 (d, J = 4.4 Hz, 3H), |
| | | | | 2.75-2.58 (m, 2H), 2.16-1.82 (m, 1H) |
| K172 | EQ | S | 800.3 | 11.08 (s, 1H), 10.71 (s, 1H), 9.77 (s, 1H), 9.05 |
| | | | | (s, 2H), 8.56-8.47 (m, 2H), 8.11 (s, 1H), 7.97 (s, |
| | | | | 1H), 7.77-7.70 (m, 1H), 7.65-7.53 (m, 2H), |
| | | | | 7.44-7.39 (m, 1H), 7.34 (t, J = 7.9 Hz, 1H), |
| | | | | 7.10-7.06 (m, 1H), 7.04-6.87 (m, 2H), 5.33 (dd, |
| | | | | J = 12.8, 5.4 Hz, 1H), 3.70 (s, 3H), 3.57-3.41 |
| | | | | (m, 3H), 3.04-2.83 (m, 3H), 2.79 (d, J = 4.4 Hz, |
| | | | | 3H), 2.75-2.58 (m, 2H), 2.49-2.47 (m, 3H), |
| | | | | 2.16-1.95 (m, 3H), 1.82-1.64 (m, 4H) |
| K171 | ER | S | 815.5 | 11.09 (s, 1H), 10.70 (s, 1H), 9.75 (s, 1H), 9.08- |
| | | | | 9.00 (m, 2H), 8.56-8.46 (m, 2H), 8.10 (s, 1H), |
| | | | | 7.95 (s, 1H), 7.73 (dd, J = 8.0, 1.6 Hz, 1H), |
| | | | | 7.67-7.51 (m, 2H), 7.41 (dd, J = 7.7, 1.7 Hz, |
| | | | | 1H), 7.32 (t, J = 7.9 Hz, 1H), 7.04-6.94 (m, 2H), |
| | | | | 6.92-6.81 (m, 1H), 5.35 (dd, J = 12.6, 5.4 Hz, |
| | | | | 1H), 3.70 (s, 3H), 3.60 (s, 3H), 3.47 (s, 2H), |
| | | | | 3.29-3.21 (m, 1H), 3.17-2.94 (m, 3H), 2.94-2.83 |
| | | | | (m, 1H), 2.79 (d, J = 4.4 Hz, 3H), 2.76-2.59 (m, |
| | | | | 2H), 2.5-2.53 (m, 1H), 2.20-2.05 (m, 4H), 2.04- |
| | | | | 1.82 (m, 2H) |
| K170 | DY | S | 815.4 | 11.05 (s, 1H), 10.70 (s, 1H), 9.75 (s, 1H), 9.08- |
| | | | | 9.00 (m, 2H), 8.55-8.47 (m, 2H), 8.13-8.09 (m, |
| | | | | 1H), 7.98-7.92 (m, 1H), 7.76-7.69 (m, 1H), |
| | | | | 7.66-7.59 (m, 1H), 7.59-7.53 (d, J = 8.5 Hz, |
| | | | | 1H), 7.42-7.36 (m, 1H), 7.35-7.27 (m, 1H), |
| | | | | 6.93-6.87 (m, 1H), 6.47-6.41 (m, 1H), 6.30-6.22 |
| | | | | (m, 1H), 5.35-5.21 (m, 1H), 3.70 (s, 3H), 3.50 |
| | | | | (s, 3H), 3.41-3.38 (m, 2H), 3.30 (s, 2H), 3.26- |
| | | | | 3.11 (m, 3H), 2.95-2.83 (m, 1H), 2.79 (d, J = |
| | | | | 4.5Hz, 3H), 2.72-2.57 (m, 2H), 2.28-2.18 (m, |
| | | | | 1H), 2.11 (s, 3H), 2.02-1.89 (m, 2H) |
| K168 | EO | S | 800.4 | 11.08 (s, 1H), 10.70 (s, 1H), 9.74 (s, 1H), 9.04 |
| | | | | (s, 2H), 8.55-8.46 (m, 2H), 8.08-8.05 (m, 1H), |
| | | | | 7.95 (s, 1H), 7.75-7.69 (m, 1H), 7.61-7.50 (m, |
| | | | | 2H), 7.41-7.36 (m, 1H), 7.29 (t, J = 7.9 Hz, 1H), |
| | | | | 7.11-7.06 (m, 1H), 7.01-6.88 (m, 2H), 5.33 (dd, |
| | | | | J = 12.8, 5.4 Hz, 1H), 3.70 (s, 3H), 3.53-3.47 |
| | | | | (m, 1H), 3.43 (s, 2H), 2.95-2.81 (m, 2H), 2.79 |
| | | | | (d, J = 4.4 Hz, 3H), 2.71-2.56 (m, 3H), 2.49- |
| | | | | 2.47 (m, 3H), 2.10-1.93 (m, 3H), 1.85-1.66 (m, |
| | | | | 2H), 1.63-1.43 (m, 2H) |
| K167 | EP | S | 800.3 | 11.09 (s, 1H), 10.70 (s, 1H), 9.75 (s, 1H), 9.04 |
| | | | | (s, 2H), 8.54-8.45 (m, 2H), 8.12-8.07 (m, 1H), |
| | | | | 7.97 (s, 1H), 7.73 (d, J = 7.9 Hz, 1H), 7.66-7.51 |
| | | | | (m, 2H), 7.45-7.29 (m, 2H), 7.06-6.94 (m, 3H), |
| | | | | 5.36 (dd, J = 12.6, 5.4 Hz, 1H), 3.70 (s, 3H), |
| | | | | 3.57 (s, 3H), 3.45 (s, 2H), 3.26-3.15 (m, 1H), |
| | | | | 2.98-2.83 (m, 3H), 2.79 (d, J = 4.4 Hz, 3H), |
| | | | | 2.76-2.56 (m, 2H), 2.17-2.04 (m, 2H), 2.03-1.94 |
| | | | | (m, 1H), 1.85-1.68 (m, 4H) |
| K166 | FN | S | 786.3 | 11.08 (s, 1H), 10.70 (s, 1H), 9.73 (s, 1H), 9.04 |
| | | | | (s, 2H), 8.51-8.48 (m, 1H), 8.21 (s, 1H), 8.13 (d, |
| | | | | J = 2.3 Hz, 1H), 7.95 (s, 1H), 7.73 (dd, J = 8.0, |
| | | | | 1.7 Hz, 1H), 7.65 (dd, J = 8.5, 2.3 Hz, 1H), 7.55 |
| | | | | (d, J = 8.5 Hz, 1H), 7.40 (dd, J = 7.8, 1.6 Hz, |
| | | | | 1H), 7.31 (t, J = 7.9 Hz, 1H), 7.11 (d, J = 1.5 |
| | | | | Hz, 1H), 7.01 (d, J = 8.1 Hz, 1H), 6.96 (dd, J = |
| | | | | 8.1, 1.6 Hz, 1H), 5.33 (dd, J = 12.8, 5.4 Hz, 1H), |
| | | | | 3.70 (s, 3H), 3.59 (s, 2H), 3.40-3.34 (m, 2H), |
| | | | | 3.33 (s, 3H), 2.98-2.83 (m, 2H), 2.80 (d, J = 4.4 |
| | | | | Hz, 3H), 2.77-2.57 (m, 4H), 2.33-2.20 (m, 1H), |
| | | | | 2.05-1.94 (m, 1H), 1.90-1.74 (m, 1H) |
| K165 | FO | S | 786.4 | 11.08 (s, 1H), 10.70 (s, 1H), 9.75 (s, 1H), 9.04 |
| | | | | (s, 2H), 8.52-8.47 (m, 2H), 8.19-8.13 (m, 1H), |
| | | | | 7.97 (s, 1H), 7.73 (dd, J = 8.0, 1.6 Hz, 1H), 7.66 |
| | | | | (dd, J = 8.6, 2.3 Hz, 1H), 7.55 (d, J = 8.5 Hz, |
| | | | | 1H), 7.42 (dd, J = 7.8, 1.6 Hz, 1H), 7.32 (t, J = |
| | | | | 7.9 Hz, 1H), 7.17 (d, J = 7.9 Hz, 1H), 7.03 (t, J = |
| | | | | 7.8 Hz, 1H), 6.96 (d, J = 7.8 Hz, 1H), 5.36 |
| | | | | (dd, J = 12.7, 5.3 Hz, 1H), 4.07-3.97 (m, 1H), |
| | | | | 3.70 (s, 3H), 3.65-359 (m, 2H), 3.57 (s, 3H), |
| | | | | 2.98-2.92 (m, 1H), 2.90-2.82 (m, 2H), 2.80 (d, J = |
| | | | | 4.0 Hz, 3 H), 2.75-2.58 (m, 4H), 2.39-2.28 (m, |
| | | | | 1H), 2.04-1.94 (m, 1H), 1.90-1.80 (m, 1H) |
| K164 | ET | S | 815.4 | 11.04 (s, 1H), 10.70 (s, 1H), 9.75 (s, 1H), 9.06- |
| | | | | 9.02 (m, 2H), 8.54-8.45 (m, 2H), 8.13-8.09 (m, |
| | | | | 1H), 7.97 (s, 1H), 7.73 (dd, J = 8.0, 1.6 Hz, 1H), |
| | | | | 7.65-7.59 (m, 1H), 7.57-7.51 (m, 1H), 7.44-7.38 |
| | | | | (m, 1H), 7.35-7.28 (m, 1H), 6.89 (d, J = 8.6 Hz, |
| | | | | 1H), 6.74-6.72 (m, 1H), 6.53 (dd, J = 8.7, 2.3 |
| | | | | Hz, 1H), 5.26 (dd, J = 12.8, 5.4 Hz, 1H), 3.70 (s, |
| | | | | 3H), 3.66-3.58 (m, 1H), 3.55-3.45 (m, 1H), 3.37 |
| | | | | (s, 2H), 3.29 (s, 3H), 2.94-2.83 (m, 1H), 2.80 (d, |
| | | | | J = 4.4 Hz, 3H), 2.77 (s, 3H), 2.74-2.56 (m, 4H), |
| | | | | 2.48-2.38 (m, 1H), 2.18-2.03 (m, 1H), 2.02-1.90 |
| | | | | (m, 1H), 1.81-1.67 (m, 1H) |
| K162 | EB | S | 829.4 | 11.08 (s, 1H), 10.69 (s, 1H), 9.71 (s, 1H), 9.06- |
| | | | | 9.00 (m, 2H), 8.54-8.45 (m, 2H), 8.08-8.04 (m, |
| | | | | 1H), 7.96-7.91 (m, 1H), 7.72 (dd, J = 7.9, 1.7 |
| | | | | Hz, 1H), 7.62-7.49 (m, 2H), 7.40 (dd, J = 7.8, |
| | | | | 1.6 Hz, 1H), 7.30 (t, J = 7.9 Hz, 1H), 7.01-6.93 |
| | | | | (m, 2H), 6.90-6.84 (m, 1H), 5.35 (dd, J = 12.7, |
| | | | | 5.5 Hz, 1H), 3.70 (s, 3H), 3.61-3.46 (m, 5H), |
| | | | | 3.32-3.28 (m, 2H), 3.28-3.15(m, 2H), 3.06-2.96 |
| | | | | (m, 1H), 2.95-2.83 (m, 1H), 2.79 (d, J = 4.4 Hz, |
| | | | | 3H), 2.76-2.58 (m, 2H), 2.18 (s, 3H), 2.04-1.90 |
| | | | | (m, 2H), 1.87-1.76 (m, 1H), 1.73-1.57 (m, 1H), |
| | | | | 1.52-1.35 (m, 1H) |
| K161 | GV | S | 801.3 | 11.09 (s, 1H), 10.69 (s, 1H), 9.71 (s, 1H), 9.06- |
| | | | | 9.02 (m, 2H), 8.54-8.45 (m, 2H), 8.07-8.01 (m, |
| | | | | 1H), 7.93 (s, 1H), 7.71 (dd, J = 8.0, 1.7 Hz, 1H), |
| | | | | 7.58-7.47 (m, 2H), 7.40 (dd, J = 7.7, 1.6 Hz, |
| | | | | 1H), 7.31 (t, J = 7.9 Hz, 1H), 6.97-6.91 (m, 1H), |
| | | | | 6.91-6.86 (m, 1H), 6.84-6.80 (m, 1H), 5.35 (dd, |
| | | | | J = 12.6, 5.5 Hz, 1H), 3.90-3.79 (m, 1H), 3.70 |
| | | | | (s, 3H), 3.65 (s, 3H), 3.48 (s, 2H), 3.34-3.25(m, |
| | | | | 4H), 2.94-2.83 (m, 1H), 2.79 (d, J = 4.4 Hz, |
| | | | | 3H), 2.74-2.57 (m, 2H), 2.53 (s, 3H), 2.06-1.94 |
| | | | | (m, 1H) |
| K160 | EC | S | 829.4 | 11.06 (s, 1H), 10.70 (s, 1H), 9.72 (s, 1H), 9.07- |
| | | | | 9.00 (m, 2H), 8.53-8.45 (m, 2H), 8.11-8.07 (m, |
| | | | | 1H), 7.95 (s, 1H), 7.72 (dd, J = 8.0, 1.7 Hz, 1H), |
| | | | | 7.61 (dd, J = 8.6, 2.3 Hz, 1H), 7.54 (d, J = 8.5 |
| | | | | Hz, 1H), 7.38 (d, J = 7.6 Hz, 1H), 7.30 (t, J = |
| | | | | 7.9 Hz, 1H), 6.98-6.88 (m, 1H), 6.84-6.80 (m, |
| | | | | 1H), 6.69-6.58 (m, 1H), 5.28 (dd, J = 12.9, 5.4 |
| | | | | Hz, 1H), 3.70 (s, 3H), 3.67-3.44 (m, 5H), 3.41(s, |
| | | | | 2H), 3.30-3.19 (m, 2H), 2.95-2.84 (m, 1H), 2.79 |
| | | | | (d, J = 4.4 Hz, 3H), 2.75-2.56 (m, 3H), 2.21 (s, |
| | | | | 3H), 2.03-1.88 (m, 2H), 1.86-1.74 (m, 1H), |
| | | | | 1.65-1.52 (m, 1H), 1.49-1.36 (m, 1H) |
| K159 | AX | S | 870.4 | 11.09 (s, 1H), 10.71 (s, 1H), 9.75 (s, 1H), 9.07- |
| | | | | 9.01 (m, 2H), 8.55-8.46 (m, 2H), 8.08-8.02 (m, |
| | | | | 1H), 7.96 (s, 1H), 7.73 (dd, J = 8.0, 1.7 Hz, 1H), |
| | | | | 7.61-7.48 (m, 2H), 7.44-7.38 (m, 1H), 7.36-7.29 |
| | | | | (m, 1H), 7.08 (dd, J = 16.7, 1.6 Hz, 1H), 7.01 |
| | | | | (dd, J = 8.1, 3.2 Hz, 1H), 6.91 (ddd, J = 19.3, |
| | | | | 8.2, 1.6 Hz, 1H), 5.34 (dd, J = 12.7, 5.3 Hz, 1H), |
| | | | | 4.34-3.92 (m, 1H), 3.70 (s, 3H), 3.56-3.47 (m, |
| | | | | 1H), 3.39 (s, 2H), 3.34 (s, 3H), 3.02-2.84 (m, |
| | | | | 1H), 2.79 (d, J = 4.4 Hz, 3H), 2.73-2.55 (m, |
| | | | | 5H), 2.41-2.31 (m, 2H), 2.15-1.85 (m, 5H), |
| | | | | 1.85-1.72 (m, 2H), 1.51-1.36 (m, 2H) |
| K158 | BZ | S | 858.4 | 11.09 (s, 1H), 10.71 (s, 1H), 9.74 (s, 1H), 9.04- |
| | | | | 9.02 (m, 2H), 8.52-8.46 (m, 2H), 8.08-8.05 (m, |
| | | | | 1H), 8.00-7.97 (m, 1H), 7.72 (d, J = 7.9 Hz, |
| | | | | 1H), 7.59-7.50 (m, 2H), 7.43-7.37 (m, 1H), |
| | | | | 7.34-7.27 (m, 1H), 7.11-6.94 (m, 3H), 5.36 (dd, |
| | | | | J = 12.8, 5.3 Hz, 1H), 4.12-3.87 (m, 1H), 3.69 |
| | | | | (s, 3H), 3.60-3.50 (m, 3H), 3.40-3.36 (m, 2H), |
| | | | | 3.35-3.33 (s, 3H), 2.95-2.82 (m, 1H), 2.79 (d, J = |
| | | | | 4.4 Hz, 3H), 2.75-2.54 (m, 4H), 2.47-2.26 (m, |
| | | | | 2H), 2.11 (s, 3H), 2.03-1.91 (m, 3H), 1.73-1.63 |
| | | | | (m, 2H) |
| K157 | IC | S | 887.4 | 11.06 (s, 1H), 10.71 (s, 1H), 9.74 (s, 1H), 9.05- |
| | | | | 9.01 (m, 2H), 8.52-8.46 (m, 2H), 8.09-8.05 (m, |
| | | | | 1H), 7.99 (s, 1H), 7.72 (dd, J = 8.0, 1.7 Hz, 1H), |
| | | | | 7.62-7.49 (m, 2H), 7.41 (dd, J = 7.8, 1.6 Hz, |
| | | | | 1H), 7.31 (t, J = 7.9 Hz, 1H), 6.88 (d, J = 8.6 |
| | | | | Hz, 1H), 6.80-6.76 (m, 1H), 6.57 (dd, J = 8.6, |
| | | | | 2.3 Hz, 1H), 5.27 (dd, J = 12.8, 5.3 Hz, 1H), |
| | | | | 3.69 (s, 3H), 3.48-3.38 (m, 3H), 3.37-3.35(m, |
| | | | | 2H), 3.33-3.31(m, 2H), 3.28 (s, 3H), 2.95-2.83 |
| | | | | (m, 1H), 2.82-2.72 (m, 5H), 2.72-2.57 (m, 2H), |
| | | | | 2.41-2.34 (m, 2H), 2.14 (s, 3H), 2.02-1.92 (m, |
| | | | | 1H), 1.90-1.80 (m, 2H), 1.72-1.60 (m, 2H), |
| | | | | 1.55-1.43 (m, 2H) |
| K156 | AS | S | 800.4 | 11.06 (s, 1H), 10.69 (s, 1H), 9.71 (s, 1H), 9.04 |
| | | | | (s, 2H), 8.49 (d, J = 3.2 Hz, 2H), 8.08 (d, J = 2.2 |
| | | | | Hz, 1H), 7.93 (s, 1H), 7.78-7.67 (m, 1H), 7.65- |
| | | | | 7.59 (m, 1H), 7.56 (d, J = 8.6 Hz, 1H), 7.44-7.36 |
| | | | | (m, 1H), 7.34-7.25 (m, 1H), 7.16-6.57 (m, 3H), |
| | | | | 5.61-5.13 (m, 1H), 3.70 (s, 3H), 3.58-3.35 (m, |
| | | | | 6H), 3.09-2.83 (m, 3H), 2.79 (d, J = 4.4 Hz, |
| | | | | 3H), 2.73-2.55 (m, 2H), 2.15-1.91 (m, 3H), 1.88 |
| | | | | (d, J = 11.3 Hz, 1H), 1.80-1.51 (m, 3H) |
| K155 | W | S | 829.4 | 11.06 (s, 1H), 10.71 (s, 1H), 9.73 (s, 1H), 9.03 |
| | | | | (s, 2H), 8.58-8.37 (m, 2H), 8.08 (d, J = 2.3 Hz, |
| | | | | 1H), 7.96 (s, 1H), 7.73 (dd, J = 8.1, 1.7 Hz, 1H), |
| | | | | 7.62-7.52 (m, 2H), 7.40 (dd, J = 7.7, 1.7 Hz, |
| | | | | 1H), 7.32 (t, J = 7.8 Hz, 1H), 6.93 (d, J = 8.6 |
| | | | | Hz, 1H), 6.84 (d, J = 2.5 Hz, 1H), 6.64 (dd, J = |
| | | | | 8.7, 2.3 Hz, 1H), 5.29 (dd, J = 12.9, 5.4 Hz, 1H), |
| | | | | 3.70 (s, 3H), 3.65 (d, J = 11.3 Hz, 2H), 3.52 (s, |
| | | | | 2H), 3.31 (s, 3H), 2.94-2.85 (m, 1H), 2.79 (d, J = |
| | | | | 4.4 Hz, 3H), 2.75-2.57 (m, 3H), 2.12 (d, J = |
| | | | | 3.4 Hz, 3H), 2.02-1.97 (m, 1H), 1.89-1.82 (m, |
| | | | | 2H), 1.71-1.62 (m, 2H), 1.26-1.21 (m, 1H), |
| | | | | 0.88-0.80 (m, 1H) |
| K154 | DX | S | 801.3 | 11.07 (s, 1H), 10.69 (s, 1H), 9.74 (s, 1H), 9.06- |
| | | | | 9.02 (m, 2H), 8.54-8.44 (m, 2H), 8.11-8.07 (m, |
| | | | | 1H), 7.93 (s, 1H), 7.73 (dd, J = 8.0, 1.7 Hz, 1H), |
| | | | | 7.63-7.53 (m, 2H), 7.41 (dd, J = 7.8, 1.6 Hz, |
| | | | | 1H), 7.32 (t, J = 7.9 Hz, 1H), 6.97 (t, J = 8.0 Hz, |
| | | | | 1H), 6.75 (d, J = 7.9 Hz, 1H), 6.69 (d, J = 8.2 |
| | | | | Hz, 1H), 5.32 (dd, J = 12.7, 5.4 Hz, 1H), 3.96- |
| | | | | 3.86 (m, 2H), 3.70 (s, 3H), 3.67-3.61 (m, 2H), |
| | | | | 3.57 (s, 2H), 3.40-3.36 (m, 4H), 2.94-2.84 (m, |
| | | | | 1H), 2.79 (d, J = 4.5 Hz, 3H), 2.73-2.57 (m, |
| | | | | 2H), 2.03 (s, 3H), 2.01-1.93 (m, 1H) |
| K153 | DZ | S | 801.4 | 11.05 (s, 1H), 10.70 (s, 1H), 9.82 (s, 1H), 9.07- |
| | | | | 8.95 (m, 2H), 8.59-8.43 (m, 2H), 8.11 (s, 1H), |
| | | | | 7.96-7.83 (m, 1H), 7.72 (d, J = 8.0 Hz, 1H), |
| | | | | 7.66-7.51 (m, 2H), 7.43-7.37 (m, 1H), 7.35-7.38 |
| | | | | (m, 1H), 6.92 (d, J = 8.4 Hz, 1H), 6.35 (s, 1H), |
| | | | | 6.14 (d, J = 8.5 Hz, 1H), 5.28 (dd, J = 12.9, 5.4 |
| | | | | Hz, 1H), 4.03-3.91 (m, 2H), 3.70 (s, 3H), 3.64- |
| | | | | 3.54 (m, 1H), 3.52-3.38 (m, 2H), 3.34 (s, 2H), |
| | | | | 3.29 (s, 3H), 2.94-2.83 (m, 1H), 2.79 (d, J = 4.3 |
| | | | | Hz, 3H), 2.74-2.56 (m, 2H), 2.09 (s, 3H), 2.01- |
| | | | | 1.93 (m, 1H) |
| K151 | ED | S | 854.5 | 11.13 (s, 1H), 10.70 (s, 1H), 9.74 (s, 1H), 9.06- |
| | | | | 9.02 (m, 2H), 8.53-8.46 (m, 2H), 8.08-8.04 (m, |
| | | | | 1H), 7.95 (s, 1H), 7.73 (dd, J = 8.0, 1.7 Hz, 1H), |
| | | | | 7.62-7.50 (m, 2H), 7.41 (dd, J = 7.7, 1.7 Hz, |
| | | | | 1H), 7.32 (t, J = 7.9 Hz, 1H), 7.20-7.14 (m, 1H), |
| | | | | 7.14-7.09 (m, 1H), 7.03 (t, J = 7.8 Hz, 1H), 5.40 |
| | | | | (dd, J = 12.7, 5.4 Hz, 1H), 4.47 (s, 2H), 3.70 (s, |
| | | | | 3H), 3.64 (s, 2H), 3.60-3.56 (m, 1H), 3.40 (s, |
| | | | | 3H), 2.95-2.83 (m, 1H), 2.79 (d, J = 4.4 Hz, |
| | | | | 3H), 2.75-2.59 (m, 4H), 2.19-2.07 (m, 2H), |
| | | | | 2.07-1.98 (m, 1H), 1.94-1.82 (m, 2H), 1.56-1.42 |
| | | | | (m, 2H) |
| K150 | AE | S | 841.4 | 11.08 (s, 1H), 10.70 (s, 1H), 9.73 (s, 1H), 9.06- |
| | | | | 9.02 (m, 2H), 8.53-8.46 (m, , 2H), 8.08 (s, 1H), |
| | | | | 7.97 (s, 1H), 7.75-7.71 (m, 1H), 7.60-7.48 (m, |
| | | | | 2H), 7.44-7.39 (m, 1H), 7.34 (t, J = 7.9 Hz, 1H), |
| | | | | 6.98-6.90 (m, 1H), 6.89-6.83 (m, 2H), 5.34 (dd, |
| | | | | J = 12.9, 5.3 Hz, 1H), 3.70 (s, 3H), 3.62 (s, 3H), |
| | | | | 3.53 (s, 2H), 3.34-3.21 (m, 4H), 3.12-2.83 (m, |
| | | | | 5H), 2.79 (d, J = 4.3 Hz, 3H), 2.72-2.57 (m, |
| | | | | 2H), 2.05-1.95 (m, 1H), 1.94-1.70 (m, 4H) |
| K149 | CN | S | 841.4 | 11.06 (s, 1H), 10.70 (s, 1H), 9.73 (s, 1H), 9.06- |
| | | | | 9.02 (m, 2H), 8.56-8.45 (m, 2H), 8.08 (d, J = 2.3 |
| | | | | Hz, 1H), 7.96 (s, 1H), 7.73 (dd, J = 8.0, 1.7 Hz, |
| | | | | 1H), 7.58 (dd, J = 8.6, 2.3 Hz, 1H), 7.52 (d, J = |
| | | | | 8.6 Hz, 1H), 7.41 (dd, J = 7.8, 1.7 Hz, 1H), 7.33 |
| | | | | (t, J = 7.9 Hz, 1H), 6.91 (d, J = 8.6 Hz, 1H), |
| | | | | 6.82 (d, J = 2.2 Hz, 1H), 6.62 (dd, J = 8.7, 2.2 |
| | | | | Hz, 1H), 5.28 (dd, J = 12.9, 5.4 Hz, 1H), 3.70 (s, |
| | | | | 3H), 3.55 (s, 2H), 3.29 (s, 3H), 3.06-2.95 (m, |
| | | | | 8H), 2.94-2.83 (m, 1H), 2.79 (d, J = 4.4 Hz, |
| | | | | 3H), 2.74-2.56 (m, 2H), 2.05-1.92 (m, 1H), |
| | | | | 1.86-1.72 (m, 4H) |
| K148 | EV | S | 858.5 | 11.07 (s, 1H), 10.74-10.66 (s, 1H), 9.72 (s, 1H), |
| | | | | 9.07-8.98 (m, 2H), 8.55-8.42 (m, 2H), 8.10-8.04 |
| | | | | (m, 1H), 8.01-7.95 (m, 1H), 7.76-7.68 (m, 1H), |
| | | | | 7.60-7.48 (m, 2H), 7.43-7.37 (m, 1H), 7.34-7.27 |
| | | | | (m, 1H), 7.09-6.96 (m, 2H), 6.93-6.83 (m, 1H), |
| | | | | 5.33 (dd, J = 12.8, 5.5, 1H), 4.17-3.77 (m, 1H), |
| | | | | 3.69 (s, 3H), 3.57-3.47 (m, 0.5H), 3.40 (s, 2H), |
| | | | | 3.38-3.29 (m, 5H), 3.03-2.84 (m, 1.5H), 2.79 (d, |
| | | | | J = 4.4 Hz, 3H), 2.75-2.54 (m, 3H), 2.44-2.35 |
| | | | | (m, 2H), 2.35-2.25 (m, 2H), 2.13 (s, 3H), 2.04- |
| | | | | 1.94 (m, 1H), 1.93-1.81 (m, 1H), 1.74-1.61 (m, 2H) |
| K147 | CO | S | 843.4 | 11.05 (s, 1H), 10.70 (s, 1H), 9.73 (s, 1H), 9.06- |
| | | | | 9.01 (m, 2H), 8.54-8.44 (m, 2H), 8.03 (s, 1H), |
| | | | | 7.95 (s, 1H), 7.75-7.68 (m, 1H), 7.58-7.47 (m, |
| | | | | 2H), 7.44-7.36 (m, 1H), 7.35-7.28 (m, 1H), |
| | | | | 6.89-6.83 (m, 1H), 6.53-6.46 (m, 1H), 6.42-6.32 |
| | | | | (m, 1H), 5.25 (dd, J = 12.8, 5.6 Hz, 1H), 3.70 (s, |
| | | | | 3H), 3.45 (s, 3H), 3.32 s, 3H), 3.29 (s, 2H), |
| | | | | 3.20-3.13 (m, 2H), 2.91-2.85 (m, 3H), 2.82-2.75 |
| | | | | (m, 5H), 2.70-2.54 (m, 2H), 2.02-1.92 (m, 1H), |
| | | | | 1.90-1.80 (m, 2H), 1.74-1.65 (m, 1H), 1.64-1.54 |
| | | | | (m, 2H) |
| K146 | CE | S | 897.4 | 11.08 (s, 1H), 10.70 (s, 1H), 9.75 (s, 1H), 9.06- |
| | | | | 9.02 (m, 2H), 8.53-8.46 (m, 2H), 8.06 (s, 1H), |
| | | | | 7.96 (s, 1H), 7.73 (d, J = 7.9 Hz, 1H), 7.60-7.50 |
| | | | | (m, 2H), 7.44-7.39 (m, 1H), 7.35-7.29 (m, 1H), |
| | | | | 7.09 (s, 1H), 6.99 (d, J = 8.0 Hz, 1H), 6.90 (d, J = |
| | | | | 8.2 Hz, 1H), 5.33 (dd, J = 12.8, 5.3 Hz, 1H), |
| | | | | 3.70 (s, 3H), 3.40 (s, 2H), 3.33 (s, 3H), 2.98- |
| | | | | 2.87 (m, 4H), 2.86-2.75 (m, 5H), 2.75-2.56 (m, |
| | | | | 2H), 2.50-2.43(m, 4H), 2.21-2.12(m, 2H), 2.04- |
| | | | | 1.89 (m, 5H), 1.75-1.63 (m, 4H), 1.56-1.46 (m, 1H) |
| K145 | AV | S | 943.4 | 11.11 (s, 1H), 10.69 (s, 1H), 9.72 (s, 1H), 9.06- |
| | | | | 9.02 (m, 2H), 8.52-8.43 (m, 4H), 8.10-8.07 (m, |
| | | | | 1H), 7.97 (s, 1H), 7.75-7.70 (m, 1H), 7.63-7.57 |
| | | | | (m, 1H), 7.56-7.51 (m, 1H), 7.44-7.40 (m, 1H), |
| | | | | 7.39-7.37 (m, 1H), 7.33 (t, J = 7.9 Hz, 1H), |
| | | | | 7.25-7.21 (m, 1H), 7.20-7.15 (m, 1H), 5.40 (dd, |
| | | | | J = 12.6, 5.3 Hz, 1H), 3.77-3.72 (m, 4H), 3.70 |
| | | | | (s, 3H), 3.48-3.42 (m, 2H), 3.36 (s, 3H), 2.95- |
| | | | | 2.84 (m, 1H), 2.79 (d, J = 4.4 Hz, 3H), 2.75-2.61 |
| | | | | (m, 2H), 2.50-2.48 (m, 2H), 2.40-2.33 (m, 2H), |
| | | | | 2.10-1.98 (m, 1H), 1.69-1.60 (m, 2H), 1.58-1.49 |
| | | | | (m, 2H) |
| K144 | EX | S | 854.4 | 11.07 (s, 1H), 10.70 (s, 1H), 9.73 (s, 1H), 9.06- |
| | | | | 9.02 (m, 2H), 8.54-8.45 (m, 2H), 8.05-8.00 (m, |
| | | | | 1H), 7.97-7.92 (m, 1H), 7.74-7.69 (m, 1H), |
| | | | | 7.58-7.48 (m, 2H), 7.43-7.37 (m, 1H), 7.35-7.27 |
| | | | | (m, 1H), 7.01-6.93 (m, 2H), 6.86-6.78 (m, 1H), |
| | | | | 5.37-5.27 (m, 1H), 3.69 (s, 3H), 3.34 (s, 2H), |
| | | | | 3.31 (s, 3H), 2.96-2.83 (m, 1H), 2.78 (d, J = 4.3 |
| | | | | Hz, 3H), 2.75-2.57 (m, 4H), 2.45-2.40 (m, 1H), |
| | | | | 2.35-2.12 (m, 4H), 2.05-1.93 (m, 1H), 1.88-1.76 |
| | | | | (m, 2H), 1.57-1.39 (m, 6H) |
| K143 | EZ | S | 857.4 | 11.08 (s, 1H), 10.70 (s, 1H), 9.75 (s, 1H), 9.06- |
| | | | | 9.00 (m, 2H), 8.55-8.45 (m, 2H), 8.08-8.01 (m, |
| | | | | 1H), 7.98-7.92 (s, 1H), 7.76-7.68 (m, 1H), 7.61- |
| | | | | 7.50 (m, 2H), 7.45-7.37 (m, 1H), 7.36-7.28 (m, |
| | | | | 1H), 7.08 (s, 1H), 7.02-6.95 (m, 1H), 6.94-6.86 |
| | | | | (m, 1H), 5.38-5.28 (m, 1H), 3.70 (s, 3H), 3.47- |
| | | | | 3.40 (m, 4H), 3.38 (s, 2H), 3.32 (s, 3H), 2.96- |
| | | | | 2.82 (m, 3H), 2.78 (d, J = 3.8 Hz, 3H), 2.74-2.55 |
| | | | | (m, 4H), 2.30-2.20 (m, 4H), 2.04-1.93 (m, 1H) |
| K142 | CA | S | 800.4 | 11.06 (s, 1H), 10.70 (s, 1H), 9.70 (s, 1H), 9.06- |
| | | | | 9.02 (m, 2H), 8.52-8.46 (m, 2H), 8.08-8.06 (m, |
| | | | | 1H), 7.95 (s, 1H), 7.73 (dd, J = 8.0, 1.7 Hz, 1H), |
| | | | | 7.58-7.50 (m, 2H), 7.44-7.39 (m, 1H), 7.33 (t, J = |
| | | | | 7.9 Hz, 1H), 7.03-6.94 (m, 2H), 6.85-6.80 (m, |
| | | | | 1H), 5.33 (dd, J = 12.8, 5.4 Hz, 1H), 3.71 (s, |
| | | | | 3H), 3.50 (s, 2H), 3.34 (s, 3H), 3.14-3.08 (m, |
| | | | | 2H), 2.94-2.82 (m, 5H), 2.80 (d, J = 4.4 Hz, |
| | | | | 3H), 2.76-2.57 (m, 2H), 2.06-1.95 (m, 1H), 1.13 |
| | | | | (s, 3H) |
| K141 | BM | S | 870.4 | 11.07 (s, 1H), 10.70 (s, 1H), 9.72 (s, 1H), 9.04 |
| | | | | (s, 2H), 8.51-8.48 (m, 2H), 8.06-8.04 (m, 1H), |
| | | | | 7.95 (s, 1H), 7.73 (dd, J = 8.0, 1.7 Hz, 1H), |
| | | | | 7.58-7.49 (m, 2H), 7.44-7.40 (m, 1H), 7.35- |
| | | | | 7.30 (m, 1H), 7.15-6.98 (m 3H), 5.36 (dd, J = |
| | | | | 12.6, 5.4 Hz, 1H), 4.28-3.99 (m, 1H), 3.70 (s, |
| | | | | 3H), 3.62-3.52 (m, 3H), 3.37 (s, 3H), 2.99-2.84 |
| | | | | (m, 1H), 2.80 (d, J = 4.4 Hz, 3H), 2.76-2.57 (m, |
| | | | | 6H), 2.49-2.31 (m, 1H), 2.10-1.99 (m, 5H), |
| | | | | 1.85-1.76 (m, 2H), 1.52-1.36 (m, 2H) |
| K140 | CQ | S | 855.4 | 11.09 (s, 1H), 10.70 (s, 1H), 9.75 (s, 1H), 9.06- |
| | | | | 9.02 (m, 2H), 8.54-8.46 (m, 2H), 8.06 (s, 1H), |
| | | | | 7.96 (s, 1H), 7.75-7.69 (m, 1H), 7.60-7.49 (m, |
| | | | | 2H), 7.44-7.38 (m, 1H), 7.35-7.29 (m, 1H), |
| | | | | 7.21-7.15 (m, 1H), 7.08-6.94 (m, 2H), 5.38-5.30 |
| | | | | (m, 1H), 3.70 (s, 3H), 3.42 (s, 2H), 3.34 (s, 3H), |
| | | | | 3.25-3.09 (m, 2H), 2.97-2.84 (m, 1H), 2.83-2.76 |
| | | | | (m, 3H), 2.75-2.56 (m, 5H), 3.35-2.19(m, 1H), |
| | | | | 2.49-2.46 (m, 2H), 2.08-1.95 (m, 3H), 1.74-1.62 |
| | | | | (m, 2H), 1.30-1.16 (m, 2H) |
| K139 | AA | S | 974.5 | 11.11 (s, 1H), 10.70 (s, 1H), 9.74 (s, 1H), 9.04 |
| | | | | (d, J = 3.2 Hz, 2H), 8.52-8.46 (m, 2H), 8.05 (s, |
| | | | | 1H), 7.95 (s, 1H), 7.73 (d, J = 7.9 Hz, 1H), 7.55- |
| | | | | 7.51 (m, 4H), 7.44-7.39 (m, 2H), 7.35-7.30 (m, |
| | | | | 1H), 7.29-7.23 (m, 1H), 7.15-7.11 (m, 1H), |
| | | | | 7.01-6.96 (m, 2H), 5.38 (d, J = 12.7 Hz, 1H), |
| | | | | 3.74-3.68 (m, 5H), 3.41-3.37 (m, 5H), 2.97-2.87 |
| | | | | (m, 1H), 2.79 (s, 3H), 2.83-2.57 (m, 4H), 2.42- |
| | | | | 2.32 (m, 8H), 2.17-2.13 (m, 2H), 2.06-2.02 (m, |
| | | | | 1H), 1.80-1.75 (m, 2H), 1.68-1.64 (m, 1H), |
| | | | | 1.25-1.16 (m, 2H) |
| K138 | FD | S | 829.3 | 11.09 (s, 1H), 10.80-10.65 (m, 1H), 9.80 (s, 1H), |
| | | | | 9.16-9.00 (m, 2H), 8.60-8.45 (m, 2H), 8.15-8.07 |
| | | | | (m, 1H), 7.98 (s, 1H), 7.73 (d, J = 8.1 Hz, 1H), |
| | | | | 7.62-7.58 (m, 1H), 7.57-7.51 (m, 1H), 7.44-7.38 |
| | | | | (m, 1H), 7.32 (t, J = 8.4 Hz, 1H), 7.10-7.04 (m, |
| | | | | 1H), 7.04-7.00 (m, 1H), 6.93-6.87 (m, 1H), 5.34 |
| | | | | (d, J = 12.6 Hz, 1H), 3.70 (s, 3H), 3.55 (s, 2H), |
| | | | | 3.44-3.37 (m, 3H), 3.30-3.22 (m, 4H), 2.98-2.82 |
| | | | | (m, 6H), 2.81-2.77 (m, 3H), 2.76-2.66 (m, 2H), |
| | | | | 2.65-2.56 (m, 3H), 2.03-1.95 (m, 1H) |
| K137 | CR | S | 843.4 | 11.10 (s, 1H), 10.70 (s, 1H), 9.70 (s, 1H), 9.06- |
| | | | | 9.00 (m, 2H), 8.53-8.45 (m, 2H), 8.04 (s, 1H), |
| | | | | 7.95 (s, 1H), 7.75-7.68 (m, 1H), 7.60-7.47 (m, |
| | | | | 2H), 7.45-7.37 (m, 1H), 7.32 (t, J = 7.9 Hz, 1H), |
| | | | | 7.11-7.03 (m, 1H), 6.99-6.92 (m, 1H), 6.90-6.84 |
| | | | | (m, 1H), 5.38 (dd, J = 12.5, 5.4 Hz, 1H), 3.70 (s, |
| | | | | 3H), 3.68 (s, 2H), 3.46 (s, 2H), 3.34 (s, 3H), |
| | | | | 2.97-2.83 (m, 2H), 2.79 (d, J = 4.4 Hz, 3H), |
| | | | | 2.75-2.59 (m, 2H), 2.46 (s, 3H), 2.16-1.88 (m, |
| | | | | 5H), 1.80-1.64 (m, 2H), 1.56-1.38 (m, 2H) |
| K136 | CS | S | 838.4 | 11.10 (s, 1H), 10.69 (s, 1H), 9.73 (s, 1H), 9.06- |
| | | | | 9.02 (m, 2H), 8.56-8.43 (m, 2H), 8.06 (s, 1H), |
| | | | | 7.94 (s, 1H), 7.76-7.68 (m, 1H), 7.62-7.47 (m, |
| | | | | 2H), 7.43-7.36 (m, 1H), 7.32 (t, J = 7.8 Hz, 1H), |
| | | | | 7.13-7.08 (m, 1H), 7.07-7.04 (m, 1H), 7.02-6.96 |
| | | | | (m, 1H), 5.38 (dd, J = 12.7, 5.4 Hz, 1H), 3.70 (s, |
| | | | | 3H), 3.63 (s, 3H), 3.30 (s, 2H), 2.94-2.82 (m, |
| | | | | 1H), 2.79 (d, J = 4.5 Hz, 3H), 2.76-2.58 (m, |
| | | | | 2H), 2.48-2.43 (m, 4H), 2.09-1.86 (m, 3H), |
| | | | | 1.84-1.68 (m, 2H), 1.63-1.48 (m, 1H), 1.42-1.20 |
| | | | | (m, 2H) |
| K135 | CB | S | 843.5 | 11.05 (s, 1H), 10.71 (s, 1H), 9.90 (s, 1H), 9.05- |
| | | | | 9.02 (m, 2H), 8.57-8.46 (m, 2H), 8.14 (s, 1H), |
| | | | | 7.98 (s, 1H), 7.76-7.67 (m, 2H), 7.62-7.54 (m, |
| | | | | 1H), 7.43 (dd, J = 7.8, 1.6 Hz, 1H), 7.32 (t, J = |
| | | | | 7.9 Hz, 1H), 6.92 (d, J = 8.6 Hz, 1H), 6.86-6.78 |
| | | | | (m, 1H), 6.66-6.58 (m, 1H), 5.28 (dd, J = 12.9, |
| | | | | 5.3 Hz, 1H), 3.70 (s, 3H), 3.63-3.52 (m, 2H), |
| | | | | 3.30 (s, 3H), 2.95-2.84 (m, 1H), 2.80 (d, J = 4.4 |
| | | | | Hz, 3H), 2.71-2.58 (m, 4H), 2.55-2.49 (m, 2H), |
| | | | | 2.50-2.47 (m, 3H), 2.48-2.37 (m, 2H), 2.05-1.93 |
| | | | | (m, 1H), 1.89-1.74 (m, 3H), 1.36-1.19 (m, 2H) |
| K134 | CC | S | 857.4 | 11.09 (s, 1H), 10.69 (s, 1H), 9.72 (s, 1H), 9.05- |
| | | | | 9.01 (m, 2H), 8.54-8.42 (m, 2H), 8.15 (s, 1H), |
| | | | | 8.05-8.00 (m, 1H), 7.74-7.67(m, 1H), 7.57-7.46 |
| | | | | (m, 2H), 7.38-7.33 (m, 1H), 7.31-7.23(m, 1H), |
| | | | | 7.07-7.00 (m, 1H), 6.96-6.88 (m, 1H), 6.87-6.82 |
| | | | | (m, 1H), 5.38-5.31 (m, 1H), 3.69 (s, 3H), 3.64 |
| | | | | (s, 2H), 3.59 (s, 2H), 3.37 (s, 3H), 2.94-2.83 (m, |
| | | | | 1H), 2.80-2.75 (m, 5H), 2.71-2.57 (m, 2H), |
| | | | | 2.49-2.47 (m, 3H), 2.17-2.11 (m, 2H), 2.02-1.90 |
| | | | | (m, 3H), 1.73-1.62 (m, 2H), 1.59-1.46 (m, 1H), |
| | | | | 1.08-0.92 (m, 2H) |
| K194 | EE | S | 815.3 | 11.09 (s, 1H), 10.69 (s, 1H), 9.74 (s, 1H), 9.07- |
| | | | | 9.00 (m, 2H), 8.53-8.44 (m, 2H), 8.05 (s, 1H), |
| | | | | 7.94 (s, 1H), 7.72 (dd, J = 8.0, 1.7 Hz, 1H), |
| | | | | 7.59-7.49 (m, 2H), 7.41 (dd, J = 7.7, 1.7 Hz, |
| | | | | 1H), 7.32 (t, J = 7.9 Hz, 1H), 7.09 (s, 1H), 7.04 |
| | | | | (d, J = 7.9 Hz, 1H), 6.96 (d, J = 8.0 Hz, 1H), |
| | | | | 5.35 (dd, J = 12.6, 5.5 Hz, 1H), 3.70 (s, 3H), |
| | | | | 3.49 (s, 2H), 3.40 (s, 3H), 3.26 (s, 2H), 2.95- |
| | | | | 2.84 (m, 1H), 2.79 (d, J = 4.5 Hz, 3H), 2.72-2.57 |
| | | | | (m, 2H), 2.44-2.30 (m, 8H), 2.06-1.96 (m, 1H) |
| K197 | DW | DV | 800.6 | 11.09 (s, 1H), 10.69 (s, 1H), 9.64 (s, 1H), 9.06- |
| | | | | 9.02 (m, 2H), 8.50-8.45 (m, 2H), 8.07-8.04 (m, |
| | | | | 1H), 7.95 (s, 1H), 7.72 (dd, J = 8.0, 1.7 Hz, 1H), |
| | | | | 7.57 (dd, J = 8.6, 2.4 Hz, 1H), 7.49 (d, J = 8.6 |
| | | | | Hz, 1H), 7.41 (dd, J = 7.8, 1.7 Hz, 1H), 7.33 (t, |
| | | | | J = 7.9 Hz, 1H), 7.15-7.12 (m, 1H), 7.09-6.99 |
| | | | | (m, 2H), 5.36 (dd, J = 12.7, 5.4 Hz, 1H), 3.70 (s, |
| | | | | 3H), 3.53 (s, 2H), 3.36 (s, 3H), 3.00-2.86 (m, |
| | | | | 3H), 2.79 (d, J = 4.4 Hz, 3H), 2.75-2.59 (m, |
| | | | | 2H), 2.55-2.53 (m, 1H), 2.11-1.96 (m, 3H), |
| | | | | 1.77-1.58 (m, 4H) |
| K199 | CP | S | 772.4 | 11.09 (s, 1H), 10.70 (s, 1H), 9.74 (s, 1H), 9.07- |
| | | | | 9.01 (m, 2H), 8.54-8.46 (m, 2H), 8.13-8.07 (m, |
| | | | | 1H), 7.96 (s, 1H), 7.73 (dd, J = 7.9, 1.7 Hz, 1H), |
| | | | | 7.64-7.58 (m, 1H), 7.56-7.51 (m, 1H), 7.43-7.38 |
| | | | | (m, 1H), 7.33 (t, J = 7.9 Hz, 1H), 7.16 (s, 1H), |
| | | | | 7.08-6.97 (m, 2H), 5.35 (dd, J = 12.7, 5.5 Hz, |
| | | | | 1H), 3.70 (s, 3H), 3.63 (m, 2H), 3.56 (s, 2H), |
| | | | | 3.34 (s, 3H), 3.30-3.26 (m, 1H), 3.18-3.12 (m, |
| | | | | 2H), 2.98-2.84 (m, 1H), 2.79 (d, J = 4.4 Hz, |
| | | | | 3H), 2.75-2.56 (m, 2H), 2.05-1.92 (m, 1H) |
| K700 | EA | S | 829.4 | 11.08 (s, 1H), 10.69 (s, 1H), 9.71 (s, 1H), 9.06- |
| | | | | 9.02 (m, 2H), 8.52-8.45 (m, 2H), 8.05-8.01 (m, |
| | | | | 1H), 7.94 (s, 1H), 7.72 (dd, J = 8.0, 1.7 Hz, 1H), |
| | | | | 7.59-7.50 (m, 2H), 7.40 (dd, J = 7.8, 1.6 Hz, |
| | | | | 1H), 7.31 (t, J = 7.9 Hz, 1H), 7.01-6.94 (m, 2H), |
| | | | | 6.91-6.86 (m, 1H), 5.34 (dd, J = 12.7, 5.4 Hz, |
| | | | | 1H), 3.70 (s, 3H), 3.59 (s, 3H), 3.36 (s, 2H), |
| | | | | 3.31-3.27 (m, 2H), 2.94-2.83 (m, 2H), 2.82-2.79 |
| | | | | (m, 5H), 2.71-2.58 (m, 5H), 2.05-1.86 (m, 3H), |
| | | | | 1.55-1.36 (m, 2H) |
| K701 | FB | S | 839.4 | 11.10 (s, 1H), 10.69 (s, 1H), 9.73 (s, 1H), 9.06- |
| | | | | 9.01 (m, 2H), 8.52-8.46 (m, 2H), 8.08-8.04 (m, |
| | | | | 1H), 7.94 (s, 1H), 7.72 (dd, J = 8.0, 1.7 Hz, 1H), |
| | | | | 7.60-7.50 (m, 2H), 7.41-7.36 (m, 1H), 7.36-7.27 |
| | | | | (m, 2H), 7.17-7.08 (m, 2H), 5.38 (dd, J = 12.8, |
| | | | | 5.3 Hz, 1H), 3.70 (s, 3H), 3.50 (s, 2H), 3.40 (s, |
| | | | | 2H), 3.34 (s, 3H), 2.96-2.83 (m, 1H), 2.79 (d, J = |
| | | | | 4.4 Hz, 3H), 2.74-2.59 (m, 2H), 2.58-2.52 (m, |
| | | | | 4H), 2.44-2.37 (m, 4H), 2.07-1.97 (m, 1H) |
| K704 | V | S | 829.4 | 11.09 (s, 1H), 10.71 (s, 1H), 9.75 (s, 1H), 9.05 |
| | | | | (s, 2H), 8.53-8.50 (m, 2H), 8.12 (s, 1H), 7.99- |
| | | | | 7.95 (m, 1H), 7.77-7.70 (m, 1H), 7.65-7.59 (m, |
| | | | | 1H), 7.57-7.53 (m, 1H), 7.44-7.39 (m, 1H), |
| | | | | 7.36-7.30 (m, 1H), 7.00-6.85 (m, 3H), 5.40-5.31 |
| | | | | (m, 1H), 3.71 (s, 3H), 3.65 (s, 3H), 3.58-3.54 |
| | | | | (m, 2H), 3.20-3.16 (m, 2H), 2.95-2.84 (m, 1H), |
| | | | | 2.80 (d, J = 4.3 Hz, 3H), 2.77-2.54 (m, 3H), |
| | | | | 2.50-2.49 (m, 2H), 2.20-2.16 (m, 3H), 2.05-1.96 |
| | | | | (m, 1H), 1.91-1.86 (m, 2H), 1.85-1.66 (m, 2H) |
| K705 | AT | S | 815.4 | 11.09 (s, 1H), 10.70 (s, 1H), 9.73 (s, 1H), 9.05 |
| | | | | (s, 2H), 8.53-8.48 (m, 2H), 8.08-8.04 (m, 1H), |
| | | | | 7.96 (s, 1H), 7.77-7.69 (m, 1H), 7.61-7.50 (m, |
| | | | | 2H), 7.45-7.38 (m, 1H), 7.34-7.30 (m, 1H), |
| | | | | 6.98-6.93 (m, 2H), 6.91-6.86 (m, 1H), 5.35 (d, J = |
| | | | | 11.0, 5.0 Hz, 1H), 3.75-3.68 (m, 4H), 3.63 (s, |
| | | | | 3H), 3.56-3.50 (m, 1H), 2.92-2.82 (m, 1H), |
| | | | | 2.81-2.78 (m, 5H), 2.75-2.65 (m, 5H), 2.57 (s, |
| | | | | 3H), 2.02-1.84 (m, 3H) |
| K708 | BL | S | 854.3 | 11.11 (s, 1H), 10.70 (s, 1H), 9.73 (s, 1H), 9.04 |
| | | | | (s, 2H), 8.51-8.48 (m, 2H), 8.05 (d, J = 2.2 Hz, |
| | | | | 1H), 7.95 (s, 1H), 7.73 (dd, J = 8.0, 1.7 Hz, 1H), |
| | | | | 7.61-7.51 (m, 2H), 7.43-7.38 (m, 1H), 7.36-7.29 |
| | | | | (m, 2H), 7.17-7.10 (m, 2H), 5.39 (dd, J = 12.8, |
| | | | | 5.3 Hz, 1H), 4.39 (s, 2H), 3.70 (s, 3H), 3.60- |
| | | | | 3.50 (m, 1H), 3.39 (s, 3H), 3.34-3.32 (m, 2H), |
| | | | | 2.96-2.84 (m, 1H), 2.79 (d, J = 4.4 Hz, 3H), |
| | | | | 2.76-2.59 (m, 4H), 2.15-2.07 (m, 2H), 2.07-1.96 |
| | | | | (m, 1H), 1.92-1.86 (m, 2H), 1.53-1.47 (m, 2H) |
| K712 | CB | FJ | 769.4 | 11.06 (s, 1H), 11.02 (s, 1H), 10.19 (s, 1H), 9.09 |
| | | | | (s, 1H), 8.22 (s, 1H), 8.10 (s, 1H), 7.67-7.61 (m, |
| | | | | 1H), 7.59-7.52 (m, 1H), 7.42 (d, J = 8.2 Hz, |
| | | | | 1H), 7.26-7.17 (m, 1H), 7.12-7.03 (m, 1H), 6.91 |
| | | | | (d, J = 8.6 Hz, 1H), 6.80 (s, 1H), 6.64-6.57 (m, |
| | | | | 1H), 5.28 (dd, J = 13.0, 5.3 Hz, 1H), 3.88 (s, |
| | | | | 3H), 3.55 (d, J = 11.7 Hz, 2H), 3.42 (s, 2H), |
| | | | | 3.29 (s, 3H), 2.95-2.83 (m, 1H), 2.73-2.56 (m, |
| | | | | 4H), 2.22-2.17 (m, 2H), 2.15 (s, 3H), 2.04-1.93 |
| | | | | (m, 1H), 1.89-1.77 (m, 2H), 1.71-1.59 (m, 1H), |
| | | | | 1.25-1.10 (m, 2H) |
| K413 | AI | AG | 849.4 | 11.09 (s, 1H), 9.26 (s, 1H), 8.90-8.74 (m, 1H), |
| | | | | 8.71-8.56 (m, 1H), 8.28-8.15 (m, 1H), 8.02-7.93 |
| | | | | (m, 1H), 7.92-7.75 (m, 2H), 7.74-7.49 (m, 4H), |
| | | | | 7.49-7.34 (m, 2H), 7.33-7.21 (m, 1H), 7.20-7.09 |
| | | | | (m, 1H), 7.08-6.88 (m, 2H), 6.00-5.77 (m, 1H), |
| | | | | 5.54-5.22 (m, 1H), 4.81-4.38 (m, 1H), 3.78-3.49 |
| | | | | (m, 2H), 3.40 (s, 3H), 3.20 (s, 3H), 3.03-2.84 |
| | | | | (m, 3H), 2.85-2.69 (m, 2H), 2.69-2.55 (m, 4H), |
| | | | | 2.11-1.96 (m, 1H), 1.40-1.06 (m, 2H), 1.01-0.71 |
| | | | | (m, 2H), 0.73-0.46 (m, 1H) |
| K414 | FK | AG | 827.5 | 11.09 (s, 1H), 9.27 (s, 1H), 8.83-8.79 (m, 1H), |
| | | | | 8.58-8.55 (m, 1H), 8.28 (s, 1H), 8.21-8.17 (m, |
| | | | | 1H), 7.94 (d, J = 8.2 Hz, 1H), 7.88 (s, 1H), 7.82- |
| | | | | 7.77 (m, 1H), 7.74-7.70 (m, 1H), 7.56-7.50 (mz, |
| | | | | 2H), 7.47-7.42 (m, 1H), 7.20-7.14 (m, 1H), |
| | | | | 7.08-6.99 (m, 2H), 5.37 (dd, J = 12.6, 5.6 Hz, |
| | | | | 1H), 4.70-4.45 (m, 1H), 3.64-3.58 (m, 2H), 3.53 |
| | | | | (s, 2H), 3.52(s, 3H), 3.31-3.27 (m, 1H), 3.20- |
| | | | | 3.13 (m, 2H), 2.90 (d, J = 4.9 Hz, 3H), 2.88-2.82 |
| | | | | (m, 1H), 2.79-2.58 (m, 5H), 2.12-1.96 (m, 4H), |
| | | | | 1.74-1.61 (m, 2H), 1.30-1.17 (m, 2H), 0.95-0.81 |
| | | | | (m, 1H), 0.66-0.54 (m, 1H) |
| K415 | AI | BP | 802.4 | 11.10 (s, 1H), 9.79 (s, 1H), 8.65-8.62 (m, 1H), |
| | | | | 8.46 (d, J = 3.9 Hz, 1H), 8.37-8.33 (m, 1H), 8.05 |
| | | | | (d, J = 8.8 Hz, 2H), 7.99 (s, 1H), 7.91-7.83 (m, |
| | | | | 2H), 7.67-7.62 (m, 1H), 7.59-7.50 (m, 3H), 7.41 |
| | | | | (s, 1H), 7.30-7.25 (m, 1H), 7.14 (d, J = 8.2 Hz, |
| | | | | 1H), 7.07 (d, J = 9.7 Hz, 1H), 7.02 (d, J = 8.5 |
| | | | | Hz, 2H), 5.38 (dd, J = 12.7, 5.4 Hz, 1H), 3.64 (s, |
| | | | | 2H), 3.40 (s, 3H), 3.23-3.17 (m, 4H), 2.98-2.85 |
| | | | | (m, 1H), 2.79-2.64 (m, 3H), 2.63-2.55 (m, 4H), |
| | | | | 2.09-2.00 (m, 1H), 0.55-0.48 (m, 2H), 0.28-0.22 |
| | | | | (m, 2H) |
| K431 | EF | AG | 796.3 | 11.06 (s, 1H), 9.25 (s, 1H), 8.81 (d, J = 4.0 Hz, |
| | | | | 1H), 8.65-8.54 (m, 1H), 8.23-8.14 (m, 1H), 7.95 |
| | | | | (d, J = 8.1 Hz, 1H), 7.88 (s, 1H), 7.81 (dd, J = |
| | | | | 8.1, 2.2 Hz, 1H), 7.73 (d, J = 7.7 Hz, 1H), 7.55 |
| | | | | (dd, J = 7.5, 1.8 Hz, 1H), 7.53-7.48 (m, 1H), |
| | | | | 7.45 (t, J = 7.8 Hz, 1H), 7.12 (d, J = 7.7 Hz, |
| | | | | 1H), 7.05 (dd, J = 8.0, 1.2 Hz, 1H), 6.99 (t, J = |
| | | | | 7.8 Hz, 1H), 5.83 (s, 1H), 5.38 (dd, J = 12.6, 5.3 |
| | | | | Hz, 1H), 4.70-4.50 (m, 1H), 3.66 (s, 3H), 3.56 |
| | | | | (s, 2H), 2.94-2.85 (m, 4H), 2.75-2.68 (m, 4H), |
| | | | | 2.67-2.58 (m, 2H), 2.27-2.18 (m, 2H), 2.07-1.98 |
| | | | | (m, 1H), 1.96-1.87 (m, 2H), 1.75-1.63 (m, 2H), |
| | | | | 0.91-0.84 (m, 1H), 0.68-0.57 (m, 1H) |
| K432 | FB | AG | 811.3 | 11.11 (s, 1H), 9.29 (s, 1H), 8.80 (d, J = 4.0 Hz, |
| | | | | 1H), 8.74 (s, 1H), 8.27-8.23 (m, 1H), 8.09 (d, J = |
| | | | | 8.2 Hz, 1H), 7.98 (d, J = 8.2 Hz, 1H), 7.89 (s, |
| | | | | 1H), 7.76 (d, J = 7.7 Hz, 1H), 7.59-7.55 (m, |
| | | | | 1H), 7.54-7.49 (m, 1H), 7.48-7.45 (m, 1H), 7.19 |
| | | | | (d, J = 7.8 Hz, 1H), 7.14 (d, J = 7.9 Hz, 1H), |
| | | | | 7.07-7.00 (m, 1H), 5.83 (s, 1H), 5.40 (dd, J = |
| | | | | 12.6, 5.4 Hz, 1H), 4.69-4.48 (m, 1H), 4.31 (s, |
| | | | | 2H), 4.05-3.91 (m, 2H), 3.65 (s, 3H), 3.35 (s, |
| | | | | 2H), 3.23-3.01 (m, 4H), 2.90 (d, J = 4.7 Hz, |
| | | | | 3H), 2.88-2.84 (m, 1H), 2.79-2.71 (m, 2H), |
| | | | | 2.70-2.54 (m, 3H), 2.07-2.00 (m, 1H), 0.94-0.85 |
| | | | | (m, 1H), 0.66-0.55 (m, 1H) |
| K433 | CT | BP | 764.3 | 11.12 (s, 1H), 9.79 (s, 1H), 8.58 (s, 1H), 8.47- |
| | | | | 8.44 (m, 1H), 8.35-8.29 (m, 1H), 8.08-8.00 (m, |
| | | | | 2H), 7.98 (s, 1H), 7.86-7.79 (m, 2H), 7.66-7.61 |
| | | | | (m, 1H), 7.55-7.50 (m, 1H), 7.18-7.09 (m, 2H), |
| | | | | 7.08-6.99 (m, 2H), 5.40 (dd, J = 12.5, 5.3 Hz, |
| | | | | 1H), 3.65 (s, 3H), 3.60-3.55 (m, 4H), 3.46- |
| | | | | 3.38(m, 4H), 3.35-3.30 (m, 2H), 3.30-3.20 (m, |
| | | | | 2H), 2.94-2.83 (m, 1H), 2.76-2.62 (m, 3H), |
| | | | 856.5 | 2.08-1.97 (m, 1H), 0.54-0.46 (m, 2H), 0.27-0.20 |
| | | | | (m, 2H) |
| K435 | AK | AG | | 11.06 (s, 1H), 9.27 (s, 1H), 8.81 (d, J = 3.9 Hz, |
| | | | | 1H), 8.57 (d, J = 2.2 Hz, 1H), 8.26-8.13 (m, |
| | | | | 2H), 7.95 (d, J = 8.1 Hz, 1H), 7.88 (s, 1H), 7.82- |
| | | | | 7.77 (m, 1H), 7.75-7.70 (m, 1H), 7.57-7.50 (m, |
| | | | | 2H), 7.49-7.42 (m, 1H), 6.92 (d, J = 8.6 Hz, |
| | | | | 1H), 6.83 (d, J = 2.2 Hz, 1H), 6.67-6.59 (m, |
| | | | | 1H), 5.82 (s, 1H), 5.35-5.22 (m, 1H), 4.69-4.50 |
| | | | | (m, 1H), 3.62 (d, J = 11.7 Hz, 2H), 3.54 (s, 2H), |
| | | | | 3.30 (s, 3H), 2.93-2.84 (m, 5H), 2.78-2.54 (m, |
| | | | | 6H), 2.48-2.24 (m, 6H), 2.11-1.93 (m, 1H), 1.85 |
| | | | | (d, J = 11.9 Hz, 2H), 1.64-1.45 (m, 2H), 0.95- |
| | | | | 0.80 (m, 1H), 0.71-0.53 (m, 1H) |
| K436 | BB | AG | 796.3 | 11.12 (s, 1H), 9.27 (s, 1H), 8.81 (d, J = 4.0 Hz, |
| | | | | 1H), 8.58 (d, J = 2.1 Hz, 1H), 8.25-8.20 (m, |
| | | | | 1H), 7.95 (d, J = 8.0 Hz, 1H), 7.88 (s, 1H), 7.84- |
| | | | | 7.77 (m, 1H), 7.73 (d, J = 7.8 Hz, 1H), 7.57-7.51 |
| | | | | (m, 3H), 7.48-7.42 (m, 1H), 7.23 (s, 1H), 7.11- |
| | | | | 7.08 (m, 2H), 5.82 (s, 1H), 5.41-5.34 (m, 1H), |
| | | | | 4.69-4.49 (m, 1H), 3.56 (s, 2H), 3.31 (s, 3H), |
| | | | | 2.92-2.87 (m, 4H), 2.76-2.58 (m, 5H), 2.26-2.18 |
| | | | | (m, 2H), 2.05-1.98 (m, 1H), 1.93-1.86 (m, 2H), |
| | | | | 1.69-1.59 (m, 2H), 0.93-0.81 (m, 1H), 0.68-0.55 |
| | | | | (m, 1H) |
| K437 | BA | CD | 963.5 | 11.08 (s, 1H), 8.67-8.62 (m, 2H), 8.57-8.53 (m, |
| | | | | 1H), 8.25-8.21 (m, 1H), 8.02-7.95 (m, 2H), 7.92 |
| | | | | (s, 1H), 7.79 (d, J = 8.2 Hz, 1H), 7.56-7.48 (m, |
| | | | | 2H), 7.21 (s, 1H), 7.19 (s, 1H), 7.13 (s, 1H), |
| | | | | 7.04-6.93 (m, 2H), 6.93-6.89 (m, 2H), 6.45-6.37 |
| | | | | (m, 2H), 5.38-5.29 (m, 1H), 4.98-4.78 (m, 1H), |
| | | | | 3.78 (s, 2H), 3.66-3.57 (m, 5H), 3.31 (s, 3H), |
| | | | | 3.09-2.97 (m, 2H), 2.91-2.85 (m, 4H), 2.83-2.70 |
| | | | | (m, 2H), 2.69-2.56 (m, 2H), 2.54 (d, J = 9.1 Hz, |
| | | | | 1H), 2.32-2.25 (m, 4H), 2.07-1.90 (m, 3H), |
| | | | | 1.82-1.79 (m, 1H), 1.58-1.53 (m, 2H), 1.32-1.20 |
| | | | | (m, 1H), 1.18-0.93 (m, 1H) |
| K454 | AC | CD | 948.3 | 11.13 (s, 1H), 8.68-8.61 (m, 3H), 8.09-7.99 (m, |
| | | | | 2H), 7.96-7.87 (m, 2H), 7.63 (d, J = 7.9 Hz, |
| | | | | 2H), 7.57-7.50 (m, 2H), 7.48-7.46 (m, 1H), |
| | | | | 7.36-7.27 (m, 3H), 7.18 (d, J = 8.3 Hz, 1H), |
| | | | | 6.46-6.35 (m, 2H), 5.41 (dd, J = 12.8, 5.4 Hz, |
| | | | | 1H), 4.99-4.75 (m, 1H),3.91 (s, 2H), 3.58- |
| | | | | 3.50(m, 2 H), 3.40 (s, 3H), 3.37-3.31 (m, 2H), |
| | | | | 3.26-3.07 (m, 4H), 3.03-2.91 (m, 1H), 2.93-2.85 |
| | | | | (m, 4H), 2.79-2.54 (m, 4H), 2.22-2.14(m, 2H), |
| | | | | 2.09-1.90 (m, 3H), 1.69-1.48 (m, 4H), 1.31-1.15 |
| | | | | (m, 1H), 1.05-0.90 (m, 1H) |
| K455 | BR | AG | 864.4 | 11.12 (s, 1H), 9.30 (s, 1H), 8.83-8.79 (m, 2H), |
| | | | | 8.68-8.65 (m, 1H), 8.24-8.21 (m, 1H), 8.15 (dd, |
| | | | | J = 8.7, 2.6 Hz, 1H), 8.02-7.98 (m, 1H), 7.97- |
| | | | | 7.93 (m, 1H), 7.92-7.86 (m, 2H), 7.76-7.73 (m, |
| | | | | 1H), 7.64-7.60 (m, 1H), 7.57-7.51 (m, 2H), |
| | | | | 7.48-7.41 (m, 2H), 7.24 (d, J = 8.2 Hz, 1H), 5.83 |
| | | | | (s, 1H), 5.42 (dd, J = 12.9, 5.4 Hz, 1H), 4.72- |
| | | | | 4.48 (m, 1H), 4.00-3.89 (m, 2H), 3.73 (s, 2H), |
| | | | | 3.42 (s, 3H), 2.90 (d, J = 4.8 Hz, 3H), 2.88-2.84 |
| | | | | (m, 2H), 2.82-2.70 (m, 3H), 2.69-2.61 (m, 2H), |
| | | | | 2.37-2.31 (m, 1H), 2.12-1.99 (m, 1H), 0.96-0.82 |
| | | | | (m, 1H), 0.73-0.53 (m, 1H) |
| K457 | BR | CD | 881.3 | 11.12 (s, 1H), 8.81 (d, J = 2.5 Hz, 1H), 8.69- |
| | | | | 8.59 (m, 3H), 8.16 (dd, J = 8.7, 2.6 Hz, 1H), |
| | | | | 8.05 (dd, J = 8.3, 2.3 Hz, 1H), 8.00 (dd, J = 7.3, |
| | | | | 1.8 Hz, 1H), 7.97-7.94 (m, 1H), 7.92 (s, 1H), |
| | | | | 7.89-7.86 (m, 1H), 7.62 (d, J = 1.7 Hz, 1H), 7.58 |
| | | | | (dd, J = 7.1, 1.8 Hz, 1H), 7.53-7.48 (m, 1H), |
| | | | | 7.44 (dd, J = 8.3, 1.7 Hz, 1H), 7.26-7.22 (m, |
| | | | | 1H), 6.46-6.37 (m, 2H), 5.42 (dd, J = 12.8, 5.4 |
| | | | | Hz, 1H), 4.99-4.76 (m, 1H), 3.98 (t, J = 5.4 Hz, |
| | | | | 2H), 3.80 (s, 2H), 3.05-2.95 (m, 2H), 2.94-2.90 |
| | | | | (m, 3H), 2.88 (d, J = 4.8 Hz, 3H), 2.82-2.73 (m, |
| | | | | 1H), 2.72-2.61 (m, 2H), 2.35-2.31 (m, 1H), |
| | | | | 2.10-2.01 (m, 1H), 1.29-1.22 (m, 3H), 1.07-0.94 |
| | | | | (m, 1H) |
| K458 | AC | BP | 884.5 | 11.12 (s, 1H), 9.78 (s, 1H), 8.58 (d, J = 2.1 Hz, |
| | | | | 1H), 8.46 (d, J = 3.9 Hz, 1H), 8.37-8.32 (m, |
| | | | | 1H), 8.09-7.97 (m, 3H), 7.88-7.78 (m, 2H), |
| | | | | 7.67-.61 (m, 1H), 7.59 (d, J = 8.0 Hz, 2H), 7.53 |
| | | | | (t, J = 7.9 Hz, 1H), 7.48-7.44 (m, 1H), 7.35- |
| | | | | 7.26 (m, 3H), 7.17 (d, J = 8.3 Hz, 1H), 7.07 (d, J = |
| | | | | 9.8 Hz, 1H), 5.40 (dd, J = 12.8, 5.4 Hz, 1H), |
| | | | | 3.54 (s, 2H), 3.40 (s, 3H), 2.97-2.87 (m, 1H), |
| | | | | 2.81-2.71 (m, 1H), 2.71-2.64 (m, 2H), 2.63- |
| | | | | 2.52 (m, 4H), 2.47-2.28 (m, 6H), 2.09-2.01 (m, |
| | | | | 1H), 1.94-1.83 (m, 4H), 1.55-1.44 (m, 2H), |
| | | | | 1.43-1.31 (m, 2H), 0.55-0.46 (m, 2H), 0.28-0.20 |
| | | | | (m, 2H) |
| K459 | BR | BP | 817.4 | 11.13 (s, 1H), 9.85 (s, 1H), 8.86-8.81 (m, 1H), |
| | | | | 8.77 (s, 1H), 8.51-8.42 (m, 2H), 8.23-8.15 (m, |
| | | | | 2H), 8.12-8.06 (m, 1H), 8.04 (s, 1H), 8.07-8.00 |
| | | | | (m, 1H), 7.98-7.87 (m, 2H), 7.69-7.60 (m, 2H), |
| | | | | 7.61-7.54 (m, 1H), 7.47-7.40 (m, 1H), 7.28-7.19 |
| | | | | (m, 1H), 7.14-7.07 (m, 1H), 5.47-5.38 (m, 1H), |
| | | | | 4.21-4.11 (m, 2H), 4.13-4.07 (m, 2H), 3.74-3.69 |
| | | | | (m, 2H), 3.42 (s, 3H), 3.36-3.30 (m, 2H), 2.98- |
| | | | | 2.86 (m, 1H), 2.83-2.67 (m, 2H), 2.65-2.60 (m, |
| | | | | 1H), 2.10-2.02 (m, 1H), 0.56-0.47 (m, 2H), |
| | | | | 0.29-0.20 (m, 2H) |
| K460 | BW | AG | 773.35 | 11.09 (s, 1H), 9.05-8.99 (m, 1H), 8.68 (s, 1H), |
| | | | | 8.65 (d, J = 4.2 Hz, 1H), 8.56 (d, J = 7.6 Hz, |
| | | | | 1H), 8.45-8.36 (m, 1H), 8.01 (dd, J = 7.4, 1.7 |
| | | | | Hz, 1H), 7.97 (d, J = 8.4 Hz, 1H), 7.92 (d, J = |
| | | | | 1.1 Hz, 1H), 7.58 (dd, J = 7.1, 1.7 Hz, 1H), |
| | | | | 7.53-7.49 (m, 1H), 7.10 (s, 1H), 7.05 (d, J = 8.1 |
| | | | | Hz, 1H), 6.97 (d, J = 8.2 Hz, 1H), 6.44 (t, J = |
| | | | | 7.2 Hz, 1H), 6.41 (s, 1H), 5.36 (dd, J = 12.6, 5.4 |
| | | | | Hz, 1H), 4.97-4.77 (m, 1H), 3.85-3.69 (m, 2H), |
| | | | | 3.47 (s, 2H), 3.34 (s, 3H), 3.04-2.98 (m, 1H), |
| | | | | 2.93-2.86 (m, 4H), 2.84-2.56 (m, 3H), 2.47- |
| | | | | 2.22 (m, 12H), 2.08-1.97 (m, 1H), 1.94-1.79 (m, |
| | | | | 4H), 1.68-1.50 (m, 3H), 1.44-1.19 (m, 4H), |
| | | | | 1.05-0.89 (m, 3H) |
| K461 | BA | AG | 946.4 | 11.08 (s, 1H), 9.31 (s, 1H), 8.81 (d, J = 3.9 Hz, |
| | | | | 1H), 8.69-8.64 (m, 1H), 8.26-8.23 (m, 1H), |
| | | | | 8.02-7.93 (m, 2H), 7.88 (s, 1H), 7.76-7.72 (m, |
| | | | | 1H), 7.56-7.43 (m, 3H), 7.29 (d, J = 8.2 Hz, |
| | | | | 2H), 7.23 (s, 1H), 7.07-7.00 (m, 2H), 6.97 (d, J = |
| | | | | 8.4 Hz, 2H), 5.85 (s, 1H), 5.36 (dd, J = 12.8, |
| | | | | 5.4 Hz, 1H), 4.69-4.47 (m, 1H), 3.96 (s, 2H), |
| | | | | 3.84 (s, 2H), 3.75 (d, J = 12.0 Hz, 2H), 3.45 (s, |
| | | | | 3H), 3.33-3.31 (m, 5H), 2.96-2.92 (m, 1H), 2.90 |
| | | | | (d, J = 4.9 Hz, 3H), 2.89-2.86 (m, 1H), 2.79- |
| | | | | 2.69 (m, 4H), 2.67-2.58 (m, 1H), 2.39-2.30 (m, |
| | | | | 1H), 2.11-1.89 (m, 4H), 1.80-1.64 (m, 2H), |
| | | | | 1.27-1.16 (m, 1H), 0.93-0.82 (m, 1H), 0.61 (m, 1H) |
| K463 | AC | AG | 931.5 | 11.12 (s, 1H), 9.27 (s, 1H), 8.81 (d, J = 3.9 Hz, |
| | | | | 1H), 8.57 (d, J = 2.2 Hz, 1H), 8.23-8.17 (m, |
| | | | | 1H), 7.95 (d, J = 8.1 Hz, 1H), 7.88 (s, 1H), 7.80 |
| | | | | (dd, J = 8.1, 2.3 Hz, 1H), 7.73 (d, J = 7.8 Hz, |
| | | | | 1H), 7.63-7.49 (m, 4H), 7.49-7.41 (m, 2H), |
| | | | | 7.35-7.25 (m, 3H), 7.18 (d, J = 8.3 Hz, 1H), 5.82 |
| | | | | (s, 1H), 5.40 (dd, J = 12.8, 5.5 Hz, 1H), 4.69- |
| | | | | 4.50 (m, 1H), 3.54 (s, 2H), 3.40 (s, 3H), 2.98- |
| | | | | 2.84 (m, 5H), 2.79-2.65 (m, 2H), 2.63-2.54 (m, |
| | | | | 4H), 2.44-2.32 (m, 6H), 2.08-2.01 (m, 1H), |
| | | | | 1.95-1.81 (m, 4H), 1.54-1.44 (m, 2H), 1.44-1.33 |
| | | | | (m, 2H), 0.93-0.82 (m, 1H), 0.68-0.54 (m, 1H) |
| K464 | HY | AG | 813.5 | 11.08 (s, 1H), 10.67-10.55 (m, 1H), 9.38-9.24 |
| | | | | (m, 1H), 8.90-8.74 (m, 2H), 8.29-8.26 (m, 1H), |
| | | | | 8.13-8.09 (m, 1H), 8.04 (dd, J = 8.3, 2.1 Hz, |
| | | | | 1H), 7.92-7.89 (m, 1H), 7.78 (d, J = 7.7 Hz, |
| | | | | 1H), 7.59-7.56 (m, 1H), 7.55-7.51 (m, 1H), |
| | | | | 7.51-7.46 (m, 1H), 7.06-6.95 (m, 1H), 6.90 (dd, |
| | | | | J = 7.9, 4.3 Hz, 2H), 5.92-5.81 (m, 1H), 5.36 |
| | | | | (dd, J = 12.8, 5.4 Hz, 1H), 4.69-4.64 (m, 1H), |
| | | | | 4.56-4.51 (m, 2H), 4.07 (s, 4H), 3.96-3.85 (m, |
| | | | | 1H), 3.63 (s, 3H), 3.03 (s, 2H), 2.91 (d, J = 3.6 |
| | | | | Hz, 3H), 2.80-2.70 (m, 2H), 2.69-2.59 (m, 2H), |
| | | | | 2.20-2.07 (m, 2H), 2.05-1.82 (m, 3H), 0.97-0.79 |
| | | | | (m, 1H), 0.69-0.56 (m, 1H) |
| K465 | EF | HW | 802.5 | 11.12 (s, 1H), 8.93 (s, 1H), 8.88 (d, J = 4.2 Hz, |
| | | | | 1H), 8.26 (s, 1H), 7.85 (s, 1H), 7.49-7.44 (m, |
| | | | | 1H), 7.18-7.09 (m, 2H), 7.07-7.03 (m, 1H), |
| | | | | 7.02-6.98 (m, 1H), 6.94-6.90 (m, 1H), 6.83 (s, |
| | | | | 1H), 6.64 (d, J = 8.4 Hz, 1H), 5.39 (dd, J = 12.6, |
| | | | | 5.4 Hz, 1H), 4.88-4.63 (m, 1H), 3.72-3.65 (m, |
| | | | | 4H), 3.31-3.20 (m, 4H), 2.92-2.85 (m, 4H), |
| | | | | 2.78-2.60 (m, 6H), 2.17-2.00 (m, 4H), 1.93-1.63 |
| | | | | (m, 7H), 1.17-1.09 (m, 3H), 0.91-0.77 (m, 1H) |
| K466 | HU | HT | 855.4 | 11.08 (s, 1H), 8.94 (s, 1H), 8.88 (d, J = 4.2 Hz, |
| | | | | 1H), 7.86 (s, 1H), 7.54 (d, J = 8.4 Hz, 2H), 7.46 |
| | | | | (q, J = 4.9 Hz, 1H), 7.42-7.40 (m, 1H), 7.29- |
| | | | | 7.25 (m, 1H), 7.21-7.11 (m, 2H), 7.01 (d, J = 8.6 |
| | | | | Hz, 2H), 6.94 (d, J = 7.9 Hz, 1H), 6.88-6.86 (m, |
| | | | | 1H), 6.65 (dd, J = 8.4, 2.3 Hz, 1H), 5.85 (s, 1H), |
| | | | | 5.39 (dd, J = 12.8, 5.4 Hz, 1H), 4.87-4.67 (m, |
| | | | | 1H), 3.78-3.72 (m, 2H), 3.40 (s, 3H), 3.16 (t, J = |
| | | | | 4.7 Hz, 4H), 2.99-2.80 (m, 5H), 2.80-2.61 (m, |
| | | | | 4H), 2.54-2.48 (m, 4H), 2.23 (d, J = 7.1 Hz, |
| | | | | 2H), 2.12-2.04 (m, 1H), 1.92-1.68 (m, 3H), |
| | | | | 1.32-1.09 (m, 3H), 0.87-0.77 (m, 1H) |
| K467 | HS | AG | 755.3 | 10.52 (s, 1H), 9.43 (s, 1H), 9.27 (s, 1H), 8.81 (d, |
| | | | | J = 4.0 Hz, 1H), 8.66 (d, J = 2.1 Hz, 1H), 8.53 |
| | | | | (s, 1H), 8.28 (s, 1H), 8.22-8.19 (m, 1H), 8.01- |
| | | | | 7.97 (m, 1H), 7.90-7.87 (m, 2H), 7.78-7.68 (m, |
| | | | | 2H), 7.64-7.60 (m, 1H), 7.59-7.49 (m, 2H), |
| | | | | 7.48-7.44 (m, 1H), 7.29 (d, J = 7.4 Hz, 1H), |
| | | | | 4.78-4.46 (m, 1H), 3.93-3.89 (m, 1H), 3.81-3.68 |
| | | | | (m, 3H), 3.21-3.11 (m, 4H), 3.03-2.92 (m, 1H), |
| | | | | 2.90 (d, J = 4.0 Hz, 3H), 2.81-2.66 (m, 6H). |
| | | | | 0.95-0.85 (m, 1H) 0.68-0.56 (m, 1H) |
| K470 | AI | LA | 873.4 | 11.08 (s, 1H), 9.22 (s, 1H), 8.69-8.61 (m, 2H), |
| | | | | 8.39 (s, 1H), 7.98 (d, J = 8.1 Hz, 1H), 7.87-7.82 |
| | | | | (m, 2H), 7.76 (d, J = 7.6 Hz, 1H), 7.63-7.36 (m, |
| | | | | 6H), 7.28 (d, J = 8.2 Hz, 1H), 7.14 (d, J = 8.2 |
| | | | | Hz, 1H), 7.02 (d, J = 8.4 Hz, 2H), 5.81 (s, 1H), |
| | | | | 5.39 (dd, J = 13.0, 5.3 Hz, 1H), 3.63 (s, 2H), |
| | | | | 3.47 (d, J = 8.5 Hz, 2H), 3.40 (s, 3H), 3.24-3.19 |
| | | | | (m, 4H), 3.14 (d, J = 8.8 Hz, 2H), 2.95-2.85 (m, |
| | | | | 5H), 2.79-2.65 (m, 2H), 2.66-2.55 (m, 4H), |
| | | | | 2.09-1.99 (m, 1H), 1.60-1.55 (m, 2H) |
| K471 | AI | LB | 858.5 | 11.11 (s, 1H), 9.24 (s, 1H), 8.85 (s, 1H), 8.64 (s, |
| | | | | 1H), 8.24 (s, 1H), 8.03-7.97 (m, 1H), 7.94 (s, |
| | | | | 1H), 7.90-7.85 (m, 1H), 7.73 (d, J = 7.4 Hz, 1H), |
| | | | | 7.60-7.54 (m, 3H), 7.53-7.45 (m, 2H), 7.42 (s, |
| | | | | 1H), 7.28 (dd, J = 8.1, 1.7 Hz, 1H), 7.14 (d, J = |
| | | | | 8.3 Hz, 1H), 7.02 (d, J = 8.4 Hz, 2H), 5.84 (s, |
| | | | | 1H), 5.39 (dd, J = 12.8, 5.4 Hz, 1H), 3.63 (s, |
| | | | | 2H), 3.40 (s, 3H), 3.26-2.15 (m, 4H), 2.97-2.88 |
| | | | | (m, 4H), 2.80-2.73 (m, 1H), 2.70-2.54 (m, 5H), |
| | | | | 2.10-2.00 (m, 1H), 1.43 (s, 6H) |
| K472 | AI | JO | 831.6 | 11.07 (s, 1H), 9.25 (s, 1H), 8.73 (d, J = 3.9 Hz, |
| | | | | 1H), 8.63 (d, J = 2.2 Hz, 1H), 8.23 (s, 1H), 7.99 |
| | | | | (d, J = 8.1 Hz, 1H), 7.90-7.81 (m, 2H), 7.76 (d, J = |
| | | | | 7.2 Hz, 1H), 7.59-7.44 (m, 5H), 7.42 (s, 1H), |
| | | | | 7.28 (d, J = 8.2 Hz, 1H), 7.14 (d, J = 8.2 Hz, |
| | | | | 1H), 7.02 (d, J = 8.4 Hz, 2H), 5.80 (s, 1H), 5.39 |
| | | | | (dd, J = 12.9, 5.4 Hz, 1H), 3.64 (s, 2H), 3.41 (s, |
| | | | | 3H), 3.25-3.18 (m, 4H), 2.98-2.86 (m, 4H), |
| | | | | 2.81-2.70 (m, 1H), 2.70-2.53 (m, 6H), 2.08-2.00 |
| | | | | (m, 1H), 0.52-0.46 (m, 2H), 0.23-0.18 (m, 2H) |
| K473 | JI | IN | 847.4 | 10.50 (s, 1H), 8.90-8.78 (m, 1H), 8.58-8.45 (m, |
| | | | | 1H), 7.97-7.91 (m, 1H), 7.76-7.51 (m, 6H), |
| | | | | 7.43-7.37 (m, 1H), 7.30-7.13 (m, 3H), 4.98-4.70 |
| | | | | (m, 1H), 4.17-3.91 (m, 2H), 3.75 (s, 2H), 3.68- |
| | | | | 3.47 (m, 10H), 3.08-2.85 (m, 7H), 2.80-2.68 (m, |
| | | | | 2H), 2.39-2.12 (m, 4H), 1.26-1.13 (m, 1H), |
| | | | | 1.03-0.87 (m, 1H) |
| K474 | IP | IN | 860.5 | 10.50 (s, 1H), 8.89 (d, J = 4.7 Hz, 1H), 8.59 (d, J = |
| | | | | 2.1 Hz, 1H), 7.96 (s, 1H), 7.84-7.67 (m, 4H), |
| | | | | 7.64 (d, J = 8.2 Hz, 1H), 7.56 (d, J = 2.0 Hz, |
| | | | | 1H), 7.40 (dd, J = 8.2, 2.0 Hz, 1H), 7.36-7.25 |
| | | | | (m, 2H), 6.02 (s, 1H), 4.98-4.76 (m, 1H), 4.15 (t, |
| | | | | J = 8.5 Hz, 2H), 3.83-3.73 (m, 1H), 3.67-3.58 |
| | | | | (m, 2H), 3.56 (s, 2H), 3.47-3.41 (m, 3H), 3.28- |
| | | | | 3.23 (m, 2H), 3.08-3.01 (m, 1H), 2.97 (d, J = 4.9 |
| | | | | Hz, 3H), 2.79-2.70 (m, 2H), 2.45-2.35 (m, 4H), |
| | | | | 1.59-1.45 (m, 6H), 1.44-1.35 (m, 2H), 1.29-1.17 |
| | | | | (m, 1H), 1.05-0.90 (m, 1H) |
| K476 | KK | IN | 792.4 | 10.52 (s, 1H), 8.91-8.85 (m, 1H), 8.62 (s, 1H), |
| | | | | 7.96 (s, 1H), 7.87-7.80 (m, 2H), 7.78-7.62 (m, |
| | | | | 3H), 7.59 (s, 1H), 7.42 (d, J = 8.3 Hz, 1H), 7.35- |
| | | | | 7.26 (m, 2H), 6.00 (s, 1H), 5.00-4.77 (m, 1H), |
| | | | | 4.14 (t, J = 8.6 Hz, 2H), 3.80-3.73 (m, 1H), |
| | | | | 3.70-3.55 (m, 8H), 3.08-3.01 (m, 1H), 2.96 (J = |
| | | | | 4.9 Hz, 3H), 2.79-2.70 (m, 2H), 2.49-2.43 (m, |
| | | | | 4H), 2.09-1.99 (m, 1H), 1.28-1.17 (m, 1H), |
| | | | | 1.03-0.91 (m, 1H) |
| K477 | AD | IN | 937.1 | 11.06 (s, 1H), 8.97-8.82 (m, 1H), 8.58 (s, 1H), |
| | | | | 7.96 (s, 1H), 7.83-7.67 (m, 4H), 7.38-7.26 (m, |
| | | | | 2H), 6.94 (d, J = 8.5 Hz, 1H), 6.83 (s, 1H), 6.62 |
| | | | | (d, J = 8.6 Hz, 1H), 6.00 (s, 1H), 5.29 (dd, J = |
| | | | | 12.9, 5.4 Hz, 1H), 5.97-4.77 (m, 1H), 4.14 (t, J = |
| | | | | 8.5 Hz, 2H), 3.52 (s, 2H), 3.44 (t, J = 8.6 Hz, |
| | | | | 2H), 3.31 (s, 3H), 3.10-3.04 (m, 4H), 2.97 (d, J = |
| | | | | 4.9 Hz, 3H), 2.91-2.83 (m, 2H), 2.81-2.59 (m, |
| | | | | 6H), 2.54-2.50 (m, 4H), 2.42-2.36 (m, 3H), |
| | | | | 2.10-1.96 (m, 4H), 1.66-1.58 (m, 2H), 1.28-1.14 |
| | | | | (m, 3H), 1.04-0.91 (m, 1H) |
| K479 | AI | IS | 879.4 | 11.13 (s, 1H), 8.82 (d, J = 4.1 Hz, 1H), 8.66 (s, |
| | | | | 1H), 8.58 (s, 1H), 7.91-7.81 (m, 3H), 7.76 (d, J = |
| | | | | 7.9 Hz, 1H), 7.57-7.53 (m, 2H), 7.52-7.45 (m, |
| | | | | 2H), 7.44-7.39 (m, 1H), 7.30-7.21 (m, 2H), 7.14 |
| | | | | (d, J = 8.3 Hz, 1H), 7.04-6.97 (m, 2H), 6.14 (s, |
| | | | | 1H), 5.39 (dd, J = 12.8, 5.4 Hz, 1H), 4.85-4.61 |
| | | | | (m, 1H), 3.64 (s, 2H), 3.52 (s, 3H), 3.40 (s, 3H), |
| | | | | 3.27-3.14 (m, 4H), 2.99-2.83 (m, 5H), 2.81-2.70 |
| | | | | (m, 2H), 2.69-2.56 (m, 4H), 2.09-2.01 (m, 1H), |
| | | | | 1.15-1.00 (m, 1H), 0.74-0.59 (m, 1H) |
| K482 | AI | LC | 861.4 | 11.10 (s, 1H), 9.27 (s, 1H), 8.66 (d, J = 5.3 Hz, |
| | | | | 2H), 8.20 (s, 1H), 7.97 (d, J = 8.0 Hz, 1H), 7.92- |
| | | | | 7.84 (m, 2H), 7.73 (d, J = 7.7 Hz, 1H), 7.60-7.54 |
| | | | | (m, 3H), 7.52-7.40 (m, 3H), 7.28 (dd, J = 8.3, |
| | | | | 1.7 Hz, 1H), 7.14 (d, J = 8.3 Hz, 1H), 7.02 (d, J = |
| | | | | 8.4 Hz, 2H), 5.84 (s, 1H), 5.39 (dd, J = 12.8, |
| | | | | 5.4 Hz, 1H), 3.64 (s, 2H), 3.40 (s, 3H), 3,27- |
| | | | | 3.17 (m, 4H), 3.12-3.09 (m, 1H), 3.07 (s, 3H), |
| | | | | 2.99-2.87 (m, 4H), 2.85-2.71 (m, 2H), 2.69-2.55 |
| | | | | (m, 5H), 2.11-1.99 (m, 1H), 0.73-0.66 (m, 1H), |
| | | | | 0.32-0.28 (m, 1H) |
| K483 | AI | LD | 845.5 | 11.12 (s, 1H), 9.25 (s, 1H), 8.86 (d, J = 7.8 Hz, |
| | | | | 1H), 8.62 (d, J = 2.1 Hz, 1H), 8.25 (d, J = 2.0 |
| | | | | Hz, 1H), 7.99 (d, J = 8.1 Hz, 1H), 7.86-7.75 (m, |
| | | | | 3H), 7.61 (dd, J = 8.0, 2.0 Hz, 1H), 7.58-7.46 |
| | | | | (m, 4H), 7.42 (d, J = 1.6 Hz, 1H), 7.27 (dd, J = |
| | | | | 8.2, 1.6 Hz, 1H), 7.14 (d, J = 8.2 Hz, 1H), 7.01 |
| | | | | (d, J = 8.5 Hz, 2H), 5.82 (s, 1H), 5.39 (dd, J = |
| | | | | 12.8, 5.4 Hz, 1H), 4.34-4.27 (m, 1H), 3.62 (s, |
| | | | | 2H), 3.41 (s, 3H), 3.24-3.18 (m, 4H), 2.98-2.86 |
| | | | | (m, 4H), 2.82-2.64 (m, 2H), 2.68-2.52 (m, 4H), |
| | | | | 2.08-1.99 (m, 3H), 1.66-1.52 (m, 3H), 1.44-1.34 |
| | | | | (m, 1H) |
| K486 | AI | KD | 772.4 | 11.13 (s, 1H), 9.16-9.12(m, 1H), 8.88-8.84 (m, |
| | | | | 1H), 7.89 (s, 1H), 7.69-7.46 (m, 4H), 7.41 (s, |
| | | | | 1H), 7.36-7.24 (m, 2H), 7.18-7.13 (m, 1H), |
| | | | | 7.07-6.93 (m, 3H). 5.82 (s, 1H), 5.48-5.31 (m, |
| | | | | 1H), 4.96-4.64 (m, 1H), 3.55 (s, 2H), 3.41 (s, |
| | | | | 3H), 3.26-3.19 (m 4H), 3.04-2.81 (m, 5H), 2.76- |
| | | | | 2.59 (m, 6H), 2.14-1.95 (m, 1H), 1.28-1.08 (m, |
| | | | | 1H), 0.91-0.68 (m, 1H) |
| K487 | AD | IS | 941.5 | 11.07 (s, 1H), 8.84-8.79 (m, 1H), 8.62-8.55 (m, |
| | | | | 2H), 7.87 (s, 1H), 7.85-7.77 (m, 2H), 7.77-7.71 |
| | | | | (m, 1H), 7.51-7.45 (m, 2H), 7.27-7.19 (m, 1H), |
| | | | | 6.97-6.91 (m, 1H), 6.83 (s, 1H), 6.66-6.59 (m, |
| | | | | 1H), 6.14 (s, 1H), 5.29 (dd, J = 13.0, 5.4 Hz, |
| | | | | 1H), 4.88-4.58 (m, 1H), 3.56-3.48 (m, 5H), |
| | | | | 3.43-3.32 (m, 4H), 3.13-3.06 (m, 4H), 2.93-2.83 |
| | | | | (m, 6H), 2.82-2.70 (m, 4H), 2.69-2.58 (m, 2H), |
| | | | | 2.43-2.35 (m, 4H), 2.11-1.96 (m, 5H), 1.67-1.60 |
| | | | | (m, 2H), 1.26-1.14 (m, 2H), 1.14-1.00 (m, 1H), |
| | | | | 0.75-0.58 (m, 1H) |
| K488 | KA | IS | 879.5 | 11.10 (s, 1H), 8.87-8.79 (m, 1H), 8.68 (s, 1H), |
| | | | | 8.59 (s, 1H), 7.91-7.82 (m, 3H), 7.80-7.73 (m, |
| | | | | 1H), 7.56-7.44 (m, 2H), 7.33-7.19 (m, 3H), |
| | | | | 7.15-6.98 (m, 4H), 6.92-6.82 (m, 1H), 6.14 (s, |
| | | | | 1H), 5.43 (dd, J = 13.0, 5.5 Hz, 1H), 4.86-4.57 |
| | | | | (m, 1H), 3.66 (s, 2H), 3.52 (s, 3H), 3.33 (s, 3H), |
| | | | | 2.94-2.91 (m, 3H), 2.91-2.83 (m, 4H), 2.81-2.66 |
| | | | | (m, 2H), 2.65-2.58 (m, 4H), 2.57-2.53 (m, 2H), |
| | | | | 2.11-2.00 (m, 1H), 1.15-1.01 (m, 1H), 0.75-0.59 |
| | | | | (m, 1H) |
| K490 | KL | KD | 793.5 | 11.06 (s, 1H), 9.09 (s, 1H), 8.83 (d, J = 4.1 Hz, |
| | | | | 1H), 7.88 (s, 1H), 7.56-7.40 (m, 2H), 7.35-7.19 |
| | | | | (m, 2H), 7.01-6.90 (m, 2H), 6.83 (s, 1H), 6.62 |
| | | | | (d, J = 8.6 Hz, 1H), 5.80 (s, 1H), 5.29 (dd, J = |
| | | | | 13.1, 5.5 Hz, 1H), 4.92-4.66 (m, 1H), 3.51 (s, |
| | | | | 2H), 3.28 (s, 3H), 3.16-2.99 (m, 4H), 2.97-2.78 |
| | | | | (m, 8H), 2.75-2.52 (m, 5H), 2.24-2.14 (m, 2H), |
| | | | | 2.07-1.93 (m, 3H), 1.76-1.65 (m, 2H), 1.60-1.47 |
| | | | | (m, 1H), 1.26-1.07 (m, 3H), 0.88-0.67 (m, 1H) |
| K494 | KA | IN | 875.5 | 11.13 (s, 1H), 8.92-8.85 (m, 1H), 8.68-8.63 (m, |
| | | | | 1H), 7.97 (s, 1H), 7.90-7.84 (m, 1H), 7.78-7.72 |
| | | | | (m, 2H), 7.72-7.67 (m, 1H), 7.38-7.24 (m, 4H), |
| | | | | 7.13-7.09 (m, 1H), 7.09-7.05 (m, 1H), 7.05-6.98 |
| | | | | (m, 2H), 6.88-6.84 (m, 1H), 6.00 (s, 1H), 5.43 |
| | | | | (dd, J = 12.7, 5.4 Hz, 1H), 5.01-4.72 (m, 1H), |
| | | | | 4.14 (t, J = 8.5 Hz, 2H), 3.64 (s, 2H), 3.45 (t, J = |
| | | | | 8.5 Hz, 2H), 3.35 (s, 3H), 3.29-3.20 (m, 4H), |
| | | | | 3.10-3.00 (m, 1H), 2.97 (d, J = 4.9 Hz, 3H), |
| | | | | 2.94-2.90 (m, 3H), 2.82-2.65 (m, 2H), 2.64-2.55 |
| | | | | (m, 2H), 2.11-2.01 (m, 1H), 1.29-1.18 (m, 1H), |
| | | | | 1.05-0.92 (m, 1H) |
| K495 | AI | CD | 866.4 | 11.12 (s, 1H), 10.10 (s, 1H), 8.77 (s, 1H), 8.68- |
| | | | | 6.52 (m, 2H), 8.17 (d, J = 18.0 Hz, 1H), 8.03 (d, |
| | | | | J = 7.5 Hz, 2H), 7.93 (s, 1H), 7.66-7.49 (m, 4H), |
| | | | | 7.44 (s, 1H), 7.30 (d, J = 8.2 Hz, 1H), 7.23-6.95 |
| | | | | (m, 4H), 6.43 (d, J = 19.2 Hz, 2H), 5.40 (dd, J = |
| | | | | 12.7, 5.5 Hz, 1H), 5.00-4.76 (m, 1H), 4.61 (d, J = |
| | | | | 20.1 Hz, 1H), 3.99-3.93 (m, 1H), 3.52 (s, 3H), |
| | | | | 3.45-3.37 (m, 4H), 3.12-3.06 (m, 1H), 3.06-2.96 |
| | | | | (m, 1H), 2.88 (d, J = 4.8 Hz, 4H), 2.81-2.61 (m, |
| | | | | 2H), 2.57-2.53 (m, 1H), 2.08-1.92 (m, 1H), |
| | | | | 1.29-1.23(m, 2H), 1.06-0.92 (m, 1H) |
| K497 | AI | KU | 861.4 | 11.10 (s, 1H), 9.22 (s, 1H), 8.75-8.61 (m, 2H), |
| | | | | 8.17 (t, J = 1.9 Hz, 1H), 7.98 (d, J = 8.1 Hz, 1H), |
| | | | | 7.90-7.82 (m, 2H), 7.74 (d, J = 7.7 Hz, 1H), |
| | | | | 7.63-7.58 (m, 1H), 7.57-7.53 (m, 2H), 7.52-7.46 |
| | | | | (m, 2H), 7.42 (d, J = 1.7 Hz, 1H), 7.29-7.26 (m, |
| | | | | 1H), 7.14 (d, J = 8.2 Hz, 1H), 7.04-7.00 (m, 2H), |
| | | | | 5.83 (s, 1H), 5.39 (dd, J = 12.8, 5.4 Hz, 1H), |
| | | | | 4.44-4.36 (m, 1H), 3.75 (dd, J = 9.0, 6.3 Hz, |
| | | | | 1H), 3.63 (s, 2H), 3.59-3.50 (m, 2H), 3.40 (s, |
| | | | | 3H), 3.36-3.33 (m, 2H), 3.24-3.18 (m, 4H), |
| | | | | 2.97-2.87 (m, 4H), 2.81-2.64 (m, 2H), 2.63-2.56 |
| | | | | (m, 4H), 2.09-1.98 (m, 1H), 1.56-1.50 (m, 1H) |
| K498 | IO | IN | 832.6 | 10.53 (s, 1H), 8.90-8.84 (m, 1H), 8.56 (s, 1H), |
| | | | | 7.96 (s, 1H), 7.78-7.70 (m, 2H), 7.70-7.64 (m, |
| | | | | 2H), 7.66-7.60 (m, 1H), 7.58-7.53 (m, 1H), |
| | | | | 7.41-7.34 (m, 1H), 7.32-7.22 (m, 2H), 5.96 (s, |
| | | | | 1H), 5.04-4.72 (m, 1H), 4.14-4.05 (m, 2H), |
| | | | | 3.81-3.70 (m, 1H), 3.64 (s, 2H), 3.56-3.52 (m, |
| | | | | 4H), 3.44-3.38 (m, 2H), 3.28-3.23 (m, 2H), 3.03 |
| | | | | (s, 4H), 2.95 (d, J = 4.9 Hz, 3H), 2.82-2.71 (m, |
| | | | | 2H), 1.78-1.65 (m, 4H), 1.29-1.15 (m, 1H), |
| | | | | 1.04-0.89 (m, 1H) |
| K505 | AI | IN | 875.5 | 11.11 (s, 1H), 8.92-8.86 (m, 1H), 8.66 (s, 1H), |
| | | | | 7.97 (s, 1H), 7.91-7.85 (m, 1H), 7.80-7.73 (m, |
| | | | | 2H), 7.73-7.68 (m, 1H), 7.59-7.53 (m, 2H), 7.42 |
| | | | | (s, 1H), 7.37-7.25 (m, 3H), 7.17-7.11 (m, 1H), |
| | | | | 7.02 (d, J = 8.4 Hz, 2H), 6.01 (s, 1H), 5.44-5.35 |
| | | | | (m, 1H), 4.96-4.76 (m, 1H), 4.19-4.11 (m, 2H), |
| | | | | 3.68-3.63 (m, 2H), 3.50-3.42 (m, 2H), 3.40 (s, |
| | | | | 3H), 3.26-3.19 (m, 2H), 3.09-3.01 (m, 1H), 2.97 |
| | | | | (d, J = 4.9 Hz, 3H), 2.95-2.86 (m, 1H), 2.81- |
| | | | | 2.64 (m, 2H), 2.64-2.59 (m, 6H), 2.08-2.01 (m, |
| | | | | 1H), 1.30-1.16 (m, 1H), 1.04-0.91 (m, 1H) |
| K506 | IO | AG | 806.4 | 10.51 (s, 1H), 9.25 (s, 1H), 8.84-8.78 (m, 1H), |
| | | | | 8.57-8.53 (m, 1H), 8.19-8.16 (m, 1H), 7.95-7.87 |
| | | | | (m, 2H), 7.79-7.74 (m, 1H), 7.73-7.68 (m, 1H), |
| | | | | 7.64 (d, J = 8.2 Hz, 1H), 7.57-7.49 (m, 3H), |
| | | | | 7.46-7.42 (m, 1H), 7.40-7.35 (m, 1H), 5.83 (s, |
| | | | | 1H), 4.70-3.50 (m, 1H), 3.80-3.72 (m, 1H), 3.64 |
| | | | | (s, 3H), 3.58-3.47 (m, 2H), 3.28-3.19 (m, 2H), |
| | | | | 3.05-2.96 (m, 4H), 2.94-2.87 (m, 3H), 2.76-2.70 |
| | | | | (m, 3H), 1.76-1.56 (m, 4H), 0.91-0.84 (m, 1H), |
| | | | | 0.66-059 (m, 1H) |
| K507 | KB | AG | 798.3b | 11.08 (s, 1H), 9.27 (s, 1H), 8.81 (d, J = 4.0 Hz, |
| | | | | 1H), 8.56 (d, J = 2.2 Hz, 1H), 8.28 (s, 1H), 8.19 |
| | | | | (t, J = 1.9 Hz, 1H), 7.94 (d, J = 8.1 Hz, 1H), 7.88 |
| | | | | (s, 1H), 7.84-7.77 (m, 1H), 7.72 (dt, J = 7.6, 1.4 |
| | | | | Hz, 1H), 7.57-7.50 (m, 2H), 7.45 (t, J = 7.9 Hz, |
| | | | | 1H), 6.96 (d, J = 4.6 Hz, 2H), 6.87 (q, J = 4.3 |
| | | | | Hz, 1H), 5.83 (s, 1H), 5.36 (dd, J = 12.6, 5.4 Hz, |
| | | | | 1H), 4.69-4.49 (m, 1H), 3.56 (s, 3H), 3,53 (s, |
| | | | | 2H), 2.93-2.88 (m, 5H), 2.88-2.81 (m, 3H), |
| | | | | 2.77-2.56 (m, 3H), 2.51-2.48 (m, 2H), 2.04-1.94 |
| | | | | (m, 3H), 1.79-1.72 (m, 2H), 1.59-1.50 (m, 2H), |
| | | | | 1.30-1.22 (m, 2H), 0.93-0.83 (m, 1H), 0.66-0.52 |
| | | | | (m, 1H) |
| K508 | AI | IW | 849.4 | 11.05 (s, 1H), 9.68 (s, 1H), 8.63 (d, J = 2.1 Hz, |
| | | | | 1H), 8.26-8.23 (m, 1H), 8.22-8.18 (m, 1H), |
| | | | | 7.98-7.84 (m, 4H), 7.79-7.72 (m, 2H), 7.56 (d, J = |
| | | | | 8.6 Hz, 2H), 7.48-7.40 (m, 2H), 7.30-7.25 (m, |
| | | | | 1H), 7.14 (d, J = 8.2 Hz, 1H), 7.04-6.99 (m, 2H), |
| | | | | 5.60 (s, 1H), 5.44-5.34 (m, 1H), 4.74-4.47 (m, |
| | | | | 1H), 3.63 (s, 2H), 3.40 (s, 3H), 3.26-3.15 (m, |
| | | | | 4H), 2.99-2.86 (m, 4H), 2.83-2.61 (m, 3H), |
| | | | | 2.61-2.55 (m, 4H), 2.09-2.00 (m, 1H), 0.99-0.80 |
| | | | | (m, 1H), 0.68-0.48 (m, 1H) |
| K510 | HY | IW | 813.4 | 11.07 (s, 1H), 9.67 (s, 1H), 8.59 (d, J = 2.2 Hz, |
| | | | | 1H), 8.24-8.17 (m, 2H), 7.97-7.87 (m, 3H), |
| | | | | 7.83-7.69 (m, 3H), 7.45 (t, J = 7.9 Hz, 1H), |
| | | | | 7.00-6.81 (m, 3H), 5.60 (s, 1H), 5.38-5.31 (m, |
| | | | | 1H), 4.73-4.53 (m, 1H), 3.71 (s, 2H), 3.63 (s, |
| | | | | 3H), 3.19-2.96 (m, 6H), 2.94 (d, J = 4.9 Hz, 3H), |
| | | | | 2.84-2.58 (m, 5H), 2.05-1.75 (m, 6H), 0.98-0.79 |
| | | | | (m, 1H), 0.68-0.54 (m, 1H) |
| K513 | KA | AG | 849.4 | 11.07 (s, 1H), 9.28 (s, 1H), 8.82 (d, J = 4.0 Hz, |
| | | | | 1H), 8.62 (s, 1H), 8.21 (s, 1H), 7.96 (d, J = 8.1 |
| | | | | Hz, 1H), 7.91-7.81 (m, 2H), 7.74 (d, J = 7.7 Hz, |
| | | | | 1H), 7.56 (d, J = 8.1 Hz, 1H), 7.53-7.42 (m, 2H), |
| | | | | 7.26 (d, J = 8.4 Hz, 2H), 7.15-7.03 (m, 2H), 7.00 |
| | | | | (d, J = 8.3 Hz, 2H), 6.86 (d, J = 7.5 Hz, 1H), |
| | | | | 5.84 (s, 1H), 5.43 (dd, J = 12.6, 5.4 Hz, 1H), |
| | | | | 4.71-4.47 (m, 1H), 3.62 (s, 2H), 3.27-3.16 (m, |
| | | | | 4H), 2.96-2.85 (m, 7H), 2.81-2.61 (m, 3H), |
| | | | | 2.60-2.53 (m, 4H), 2.09-2.01 (m, 1H), 0.92-0.82 |
| | | | | (m, 1H), 0.69-0.56 (m, 1H) |
| K514 | AA | CD | 963.6 | 11.12 (s, 1H), 8.68-8.61 (m, 2H), 8.53 (d, J = 2.2 |
| | | | | Hz, 1H), 7.99 (dd, J = 7.3, 1.8 Hz, 1H), 7.98- |
| | | | | 7.90 (m, 2H), 7.82 (d, J = 8.2 Hz, 1H), 7.59-7.47 |
| | | | | (m, 4H), 7.41 (d, J = 1.6 Hz, 1H), 7.34-7.23 (m, |
| | | | | 1H), 7.14 (d, J = 8.2 Hz, 1H), 7.00 (d, J = 8.4 |
| | | | | Hz, 2H), 6.41-6.38 (m, 2H), 5.39 (dd, J = 12.8, |
| | | | | 5.4 Hz, 1H), 5.01-4.76 (m, 1H), 3.73 (d, J = 11.9 |
| | | | | Hz, 2H), 3.60 (s, 2H), 3.40 (s, 3H), 3.05-2.92 |
| | | | | (m, 2H), 2.88 (d, J = 4.8 Hz, 3H), 2.82-2.60 (m, |
| | | | | 4H), 2.49-2.27 (m, 8H), 2.20-2.14 (m, 2H), |
| | | | | 2.08-2.00 (m, 1H), 1.83-1.75 (m, 2H), 1.70-1.63 |
| | | | | (m, 1H), 1.29-1.14 (m, 3H), 1.06-0.91 (m, 1H) |
| K517 | IR | AG | 911.5 | 11.09 (s, 1H), 9.27 (s, 1H), 8.81 (d, J = 3.9 Hz, |
| | | | | 1H), 8.55 (d, J = 2.1 Hz, 1H), 8.26 (s, 1H), 7.93 |
| | | | | (d, J = 8.1 Hz, 1H), 7.88 (s, 1H), 7.78 (dd, J = |
| | | | | 8.3, 2.2 Hz, 1H), 7.72 (d, J = 7.7 Hz, 1H), 7.58- |
| | | | | 7.48 (m, 2H), 7.45 (t, J = 7.8 Hz, 1H), 6.98 (t, J = |
| | | | | 7.9 Hz, 1H), 6.94-6.87 (m, 2H), 5.83 (s, 1H), |
| | | | | 5.35 (dd, J = 12.8, 5.4 Hz, 1H), 4.67-4.48 (m, |
| | | | | 1H), 3.61 (s, 2H), 3.52 (s, 3H), 3.44-3.37 (m, |
| | | | | 2H), 2.97-2.79 (m, 9H), 2.78-2.59 (m, 8H), |
| | | | | 2.21-1.89 (m, 5H), 1.68-1.60 (m, 2H), 1.28-1.18 |
| | | | | (m, 3H), 0.91-0.83 (m, 1H), 0.67-0.57 (m, 1H) |
| K518 | AI | HW | 855.5 | 11.13 (s, 1H), 8.94 (s, 1H), 8.89 (d, J = 4.2 Hz, |
| | | | | 1H), 7.86 (s, 1H), 7.58-7.53 (m, 2H), 7.50-7.45 |
| | | | | (m, 1H), 7.43-7.41 (m, 1H), 7.40-7.31 (m, 1H), |
| | | | | 7.30-7.24 (m, 1H), 7.22-7.12 (m, 2H), 7.01 (d, J = |
| | | | | 8.4 Hz, 2H), 6.95-6.91 (m, 1H), 6.86-6.83 (m, |
| | | | | 1H), 6.67-6.63 (m, 1H), 5.40 (dd, J = 13.0, 5.0 |
| | | | | Hz, 1H), 4.89-4.65 (m, 1H), 3.73-3.65 (m, 2H), |
| | | | | 3.41 (s, 3H), 3.21-3.16 (m, 4H), 2.99-2.87 (m, |
| | | | | 5H), 2.80-2.61 (m, 5H), 2.55-2.52 (m, 2H), 2.23 |
| | | | | (d, J = 7.0 Hz, 2H), 2.09-1.94 (m, 2H), 1.86- |
| | | | | 1.67 (m, 3H), 1.31-1.08 (m, 3H), 0.90-0.77 (m, 1H) |
| K519 | FQ | CD | 878.4 | 11.11 (s, 1H), 8.67-8.62 (m, 2H), 8.52 (s, 1H), |
| | | | | 8.01-7.97 (m, 1H), 7.95-7.87 (m, 2H), 7.82-7.79 |
| | | | | (m, 1H), 7.55-7.48 (m, 4H), 7.38 (s, 1H), 7.23 |
| | | | | (s, 1H), 7.14-7.10 (m, 1H), 6.54-6.48 (m, 2H), |
| | | | | 6.43-6.38 (m, 2H), 5.41-5.34 (m, 1H), 4.91- |
| | | | | 4.85 (m, 1H), 3.98-3.90 (m, 4H), 3.72-3.65 (m, |
| | | | | 2H), 3.42-3.37 (m, 7H), 3.04-2.96 (m, 1H), |
| | | | | 2.90-2.85 (m, 3H), 2.76-2.54 (m, 2H), 2.13-1.94 |
| | | | | (m, 1H), 1.33-1.19 (m, 3H), 1.06-0.93 (m, 1H) |
| K520 | HY | JK | 819.2 | 11.09 (s, 1H), 8.93 (s, 1H), 8.88 (d, J = 4.2 Hz, |
| | | | | 1H), 7.86 (s, 1H), 7.50-7.45 (m, 1H), 7.18-7.12 |
| | | | | (m, 1H), 7.00-6.91 (m, 2H), 6.90-6.85 (m, 2H), |
| | | | | 6.85-6.77 (m, 1H), 6.66-6.61 (m, 1H), 5.76 (s, |
| | | | | 1H), 5.35 (dd, J = 12.8, 5.3 Hz, 1H), 4.89-4.65 |
| | | | | (m, 1H), 3.71-3.64 (m, 4H), 3.62 (s, 3H), 3.31- |
| | | | | 3.07 (m, 5H), 3.07-2.80 (m, 7H), 2.76-2.58 (m, |
| | | | | 5H), 2.04-1.89 (m, 3H), 1.87-1.71 (m, 4H), |
| | | | | 1.55-1.45 (m, 1H), 1.30-1.08 (m, 3H), 0.89-0.77 |
| | | | | (m, 1H) |
| K521 | AD | CD | 928.5 | 11.06 (s, 1H), 8.67-8.64 (m, 1H), 8.62 (s, 1H), |
| | | | | 8.56-8.51 (m, 1H), 8.15 (s, 1H), 8.02-7.98 (m, |
| | | | | 1H), 7.95-7.92 (m, 2H), 7.87-7.78 (m, 1H), |
| | | | | 7.60-7.47 (m, 2H), 6.94 (d, J = 8.6 Hz, 1H), |
| | | | | 6.88-6.81 (m, 1H), 6.68-6.61 (m, 1H), 6.47-6.34 |
| | | | | (m, 2H), 5.30 (dd, J = 12.9, 5.3 Hz, 1H), 4.99- |
| | | | | 4.75 (m, 1H), 3.69 (s, 2H), 3.63-3.56 (m, 2H), |
| | | | | 3.45 (s, 3H), 3.26-3.19 (m, 2H), 3.16-3.07 (m, |
| | | | | 4H), 3.06-2.90 (m, 3H), 2.88 (d, J = 4.9 Hz, 3H), |
| | | | | 2.86-2.76 (m, 2H), 2.76-2.58 (m, 2H), 2.49- |
| | | | | 2.41 (m, 4H), 2.13-1.95 (m, 3H), 1.81-1.70 (m, |
| | | | | 2H), 1.34-1.19 (m, 3H), 1.05-0.93 (m, 1H) |
| K522 | FQ | AG | 861.4 | 11.09 (s, 1H), 9.25 (d, J = 10.1 Hz, 1H), 8.80 (d, |
| | | | | J = 10.8 Hz, 1H), 8.59-8.52 (m, 1H), 8.18 (d, J = |
| | | | | 10.0 Hz, 1H), 7.98-7.83 (m, 2H), 7.82-7.66 (m, |
| | | | | 3H), 7.58-7.33 (m, 6H), 7.24 (d, J = 8.7 Hz, 1H), |
| | | | | 7.14-7.08 (m, 1H), 6.55-6.44 (m, 2H), 5.85-5.78 |
| | | | | (m, 1H), 5.41-5.34 (m, 1H), 4.73-4.45 (m, 1H), |
| | | | | 3.95-3.87 (m, 4H), 3.64-3.57 (m, 2H), 3.41-3.37 |
| | | | | (m, 4H), 2.96-2.84 (m, 5H), 2.76-2.63 (m, 2H), |
| | | | | 2.15-1.84 (m, 2H), 0.94-0.70 (m, 2H), 0.64-0.37 |
| | | | | (m, 1H) |
| K523 | T | AG | 773.4 | 11.07 (s, 1H), 9.34-9.25 (m, 1H), 8.85-8.75 (m, |
| | | | | 2H), 8.32-8.27 (m, 1H), 8.17-8.11 (m, 1H), |
| | | | | 8.10-8.02 (m, 1H), 7.91-7.85 (m, 1H), 7.79 (d, J = |
| | | | | 7.7 Hz, 1H), 7.65-7.41 (m, 3H), 7.04-7.97 (m, |
| | | | | 1H), 6.94-6.90 (m, 1H), 6.73-6.67 (m, 1H), |
| | | | | 5.87-5.81 (m, 1H), 5.32 (dd, J = 13.1, 5.2 Hz, |
| | | | | 1H), 4.71-4.42 (m, 3H), 3.87-3.41 (m, 4H), 3.32 |
| | | | | (s, 3H), 3.31-3.15 (m, 2H), 3.11-2.83 (m, 6H), |
| | | | | 2.80-2.57 (m, 3H), 2.05-1.94 (m, 1H), 0.97-0.77 |
| | | | | (m, 1H), 0.68-0.51 (m, 1H) |
| K525 | KC | IN | 846.4 | 10.51 (s, 1H), 8.87 (d, J = 4.7 Hz, 1H), 8.58 (d, J = |
| | | | | 2.1 Hz, 1H), 7.96 (s, 1H), 7.81-7.72 (m, 2H), |
| | | | | 7.71-7.65 (m, 2H), 7.63 (d, J = 8.2 Hz, 1H), 7.55 |
| | | | | (d, J = 2.0 Hz, 1H), 7.38 (dd, J = 8.2, 2.0 Hz, |
| | | | | 1H), 7.33-7.23 (m, 2H), 5.98 (s, 1H), 4.98-4.76 |
| | | | | (m, 1H), 4.53-4.40 (m, 1H), 4.12 (t, J = 8.5 Hz, |
| | | | | 2H), 3.80-3.70 (m, 1H), 3.68 (s, 2H), 3.65-3.56 |
| | | | | (m, 2H), 3.46-3.32 (m, 4H), 3.10-2.92 (m, 7H), |
| | | | | 2.79-2.70 (m, 3H), 2.26-2.15 (m, 1H), 1.78-1.65 |
| | | | | (m, 2H), 1.61-1.50 (m, 1H), 1.28-1.16 (m, 1H), |
| | | | | 1.08-0.98 (m, 2H), 0.97-0.90 (m, 1H) |
| K526 | AI | LG | 849.5 | 11.10 (s, 1H), 8.80 (t, J = 5.8 Hz, 1H), 8.66 (d, J = |
| | | | | 1.6 Hz, 1H), 8.60 (s, 1H), 7.86-7.84 (m, 2H), |
| | | | | 7.82-7.78 (m, 2H), 7.56 (d, J = 8.5 Hz, 2H), |
| | | | | 7.50-7.47 (m, 1H), 7.47-7.41 (m, 2H), 7.29-7.23 |
| | | | | (m, 2H), 7.14 (d, J = 8.3 Hz, 1H), 7.02 (d, J = |
| | | | | 8.8 Hz, 2H), 6.12 (s, 1H), 5.39 (dd, J = 12.8, 5.4 |
| | | | | Hz, 1H), 3.65 (s, 2H), 3.54 (s, 3H), 3.43-3.39 |
| | | | | (m, 3H), 3.29-3.20 (m, 6H), 2.90 (d, J = 4.9 Hz, |
| | | | | 3H), 2.81-2.65 (m, 2H), 2.64-2.55 (m, 5H), |
| | | | | 2.09-2.00 (m, 1H), 0.99 (t, J = 7.2 Hz, 3H) |
| K528 | AI | JL | 863.5 | 11.10 (s, 1H), 8.68-8.65 (m, 1H), 8.58-8.51 (m, |
| | | | | 2H), 7.86-7.83 (m, 2H), 7.81-7.75 (m, 2H), |
| | | | | 7.57-7.49 (m, 3H), 7.45-7.40 (m, 2H), 7.30-7.22 |
| | | | | (m, 2H), 7.14 (d, J = 8.2 Hz, 1H), 7.02 (d, J = |
| | | | | 8.4 Hz, 2H), 6.11 (d, J = 1.6 Hz, 1H), 5.38 (dd, J = |
| | | | | 12.7, 5.4 Hz, 1H), 4.12-4.01 (m, 1H), 3.65 (s, |
| | | | | 2H), 3.54 (s, 3H), 3.40 (s, 3H), 3.25-3.19 (m, |
| | | | | 4H), 2.96-2.89 (m, 4H), 2.81-2.64 (m, 2H), |
| | | | | 2.64-2.56 (m, 4H), 2.09-2.01 (m, 1H), 1.00 (d, J = |
| | | | | 6.6 Hz, 6H) |
| K529 | AI | KV | 834.3 | 11.05 (s, 1H), 9.33 (d, J = 5.2 Hz, 1H), 8.68 (d, J = |
| | | | | 2.2 Hz, 1H), 8.29-8.24 (m, 2H), 8.18-8.04 (m, |
| | | | | 4H), 8.03-7.99 (m, 1H), 7.91-7.87 (m, 1H), 7.56 |
| | | | | (d, J = 8.4 Hz, 2H), 7.42 (d, J = 1.7 Hz, 1H), |
| | | | | 7.30-7.25 (m, 1H), 7.14 (d, J = 8.2 Hz, 1H), 7.02 |
| | | | | (d, J = 8.5 Hz, 2H), 6.78 (s, 1H), 5.39 (dd, J = |
| | | | | 12.7, 5.4 Hz, 1H), 5.11-4.87 (m, 1H), 3.66 (s, |
| | | | | 2H), 3.40 (s, 3H), 3.25-3.19 (m, 4H), 3.17-3.10 |
| | | | | (m, 1H), 3.07 (d, J = 4.9 Hz, 3H), 2.99-2.86 (m, |
| | | | | 1H), 2.79-2.66 (m, 1H), 2.64-2.57 (m, 3H), |
| | | | | 2.09-2.00 (m, 1H), 1.33-1.23 (m, 1H), 1.08-0.96 |
| | | | | (m, 1H) |
| K534 | AI | KI | 857.5 | 11.11 (s, 1H), 10.04 (s, 1H), 9.28 (d, J = 9.7 Hz, |
| | | | | 1H), 8.81-8.75 (m, 1H), 8.48-8.33 (m, 2H), |
| | | | | 8.16-8.05 (m, 2H), 8.03-7.93 (m, 1H), 7.85-7.74 |
| | | | | (m, 2H), 7.68-7.61 (m, 2H), 7.51 (t, J = 7.9 Hz, |
| | | | | 1H), 7.45-7.32 (m, 2H), 7.32-7.22 (m, 1H), |
| | | | | 7.19-7.04 (m, 3H), 5.91 (dd, J = 9.8, 6.6 Hz, |
| | | | | 1H), 5.39 (dd, J = 12.7, 5.4 Hz, 1H), 4.70-4.46 |
| | | | | (m, 3H), 3.95-3.87 (m, 2H), 3.57-3.46 (m, 2H), |
| | | | | 3.47-3.39 (m, 5H), 3.12-3.02 (m, 1H), 2.99-2.91 |
| | | | | (m, 4H), 2.81-2.60 (m, 2H), 2.08-2.01 (m, 1H), |
| | | | | 1.46-1.35 (m, 6H) |
| K536 | LQ | HT | 880.6 | 11.11 (s, 1H), 8.88 (d, J = 4.6 Hz, 1H), 7.94 (s, |
| | | | | 1H), 7.71-7.62 (m, 1H), 7.58-7.47 (m, 3H), 7.41 |
| | | | | (d, J = 1.7 Hz, 1H), 7.27 (dd, J = 8.2, 1.7 Hz, |
| | | | | 1H), 7.19-7.11 (m, 2H), 7.01 (d, J = 8.6 Hz, 2H), |
| | | | | 6.85 (d, J = 7.7 Hz, 1H), 5.99 (s, 1H), 5.39 (dd, J = |
| | | | | 12.8, 5.4 Hz, 1H), 5.01-4.75 (m, 1H), 4.14 (t, J = |
| | | | | 8.6 Hz, 2H), 3.76 (d, J = 12.0 Hz, 2H), 3.40 (s, |
| | | | | 3H), 3.18 (t, J = 8.4 Hz, 2H), 3.09-2.86 (m, 7H), |
| | | | | 2.82-2.61 (m, 4H), 2.59-2.52 (m, 1H), 2.25-2.19 |
| | | | | (m, 2H), 2.10-1.96 (m, 3H), 1.88-1.80 (m, 2H), |
| | | | | 1.77-1.63 (m, 5H), 1.30-1.18 (m, 3H), 1.05-0.90 |
| | | | | (m, 1H) |
| K540 | KJ | HT | 892.6 | 11.11 (s, 1H), 9.14 (d, J = 4.5 Hz, 1H), 8.15 (s, |
| | | | | 1H), 8.06-7.98 (m, 2H), 7.70 (q, J = 4.9 Hz, 1H), |
| | | | | 7.55 (d, J = 8.4 Hz, 2H), 7.42 (d, J = 1.7 Hz, |
| | | | | 1H), 7.28 (dd, J = 8.2, 1.7 Hz, 1H), 7.21 (d, J = |
| | | | | 7.3 Hz, 1H), 7.15 (t, J = 8.1 Hz, 2H), 7.05-6.99 |
| | | | | (m, 2H), 6.57 (s, 1H), 5.39 (dd, J = 12.8, 5.4 Hz, |
| | | | | 1H), 5.02-4.78 (m, 1H), 4.14 (s, 3H), 3.77 (d, J = |
| | | | | 11.8 Hz, 2H), 3.41 (s, 3H), 3.12-3.04 (m, 2H), |
| | | | | 3.01 (d, J = 4.9 Hz, 3H), 2.98-2.87 (m, 1H), |
| | | | | 2.81-2.60 (m, 4H), 2.30-2.20 (m, 2H), 2.19-2.09 |
| | | | | (m, 2H), 2.08-2.00 (m, 1H), 1.96-1.67 (m, 7H), |
| | | | | 1.34-1.20 (m, 4H), 1.13-0.98 (m, 1H), 0.90-0.79 |
| | | | | (m, 1H) |
| K541 | AI | KY | 883.4 | 11.08 (s, 1H), 8.73 (s, 1H), 8.70 (d, J = 4.1 Hz, |
| | | | | 1H), 8.67-8.64 (m, 1H), 7.91-7.84 (m, 2H), 7.76 |
| | | | | (dd, J = 7.9, 1.7 Hz, 1H), 7.64 (d, J = 8.0 Hz, |
| | | | | 1H), 7.59-7.51 (m, 3H), 7.46 (t, J = 7.8 Hz, 1H), |
| | | | | 7.42 (d, J = 1.6 Hz, 1H), 7.38 (dd, J = 7.7, 1.7 |
| | | | | Hz, 1H), 7.28 (dd, J = 8.2, 1.7 Hz, 1H), 7.14 (d, |
| | | | | J = 8.2 Hz, 1H), 7.02 (d, J = 8.5 Hz, 2H), 6.04 |
| | | | | (s, 1H), 5.39 (dd, J = 12.8, 5.4 Hz, 1H), 4.80- |
| | | | | 4.58 (m, 1H), 3.66 (s, 2H), 3.40 (s, 3H), 3.26- |
| | | | | 3.16 (m, 4H), 3.05-2.87 (m, 4H), 2.85-2.78 (m, |
| | | | | 1H), 2.77-2.65 (m, 1H), 2.64-2.56 (m, 5H), |
| | | | | 2.09-2.00 (m, 1H), 1.09-0.95 (m, 1H), 0.57-0.44 |
| | | | | (m, 1H) |
| K542 | AI | LF | 875.5 | 11.00 (s, 1H), 8.73-8.64 (m, 2H), 8.57 (s, 1H), |
| | | | | 7.87-7.85 (m, 2H), 7.80 (d, J = 1.1 Hz, 1H), 7.74 |
| | | | | (dd, J = 7.9, 1.7 Hz, 1H), 7.59-7.50 (m, 3H), |
| | | | | 7.50-7.40 (m, 2H), 7.30-7.24 (m, 2H), 7.14 (d, J = |
| | | | | 8.3 Hz, 1H), 7.06-6.97 (m, 2H), 6.10 (s, 1H), |
| | | | | 5.39 (dd, J = 12.8, 5.4 Hz, 1H), 3.65 (s, 2H), |
| | | | | 3.52 (s, 3H), 3.41 (s, 3H), 3.27-3.20 (m, 4H), |
| | | | | 2.97-2,86 (m, 4H), 2.79-2.65 (m, 1H), 2.64-2.57 |
| | | | | (m, 5H), 2.49-2.44 (m, 1H), 2.11-2.00 (m, 1H), |
| | | | | 0.99 (d, J = 6.1 Hz, 3H), 0.70-0.63 (m, 1H), |
| | | | | 0.48-0.40 (m, 2H) |
| K544 | KM | IN | 867.6 | 11.06 (s, 1H), 8.89 (d, J = 4.7 Hz, 1H), 8.63- |
| | | | | 8.58 (m, 1H), 7.96 (s, 1H), 7.85-7.79 (m, 1H), |
| | | | | 7.78-7.66 (m, 3H), 7.36-7.25 (m, 2H), 6.93 (d, J = |
| | | | | 8.6 Hz, 1H), 6.83 (d, J = 2.2 Hz, 1H), 6.64 |
| | | | | (dd, J = 8.7, 2.1 Hz, 1H), 6.02 (s, 1H), 5.29 (dd, |
| | | | | J = 12.8, 5.4 Hz, 1H), 4.99-4.77 (m, 1H), 4.15 (t, |
| | | | | J = 8.4 Hz, 2H), 3.57 (s, 2H), 3.45 (t, J = 8.5 Hz, |
| | | | | 2H), 3.33 (s, 3H), 3.12-3.01 (m, 5H), 2.97 (d, J = |
| | | | | 4.9 Hz, 3H), 2.94-2.83 (m, 1H), 2.75-2.57 (m, |
| | | | | 2H), 2.46-2.39 (m, 4H), 2.03-1.95 (m, 1H), |
| | | | | 1.62-1.55 (m, 4H), 1.54-1.48 (m, 4H), 1.26-1.19 |
| | | | | (m, 1H), 1.05-0.90 (m, 1H) |
| K551 | AI | JJ | 881.6 | 11.11 (s, 1H), 8.93 (d, J = 4.8 Hz, 1H), 7.94 (s, |
| | | | | 1H), 7.70-7.63 (m, 1H), 7.56 (d, J = 8.5 Hz, 2H), |
| | | | | 7.42 (d, J = 1.7 Hz, 1H), 7.32-7.25 (m, 2H), |
| | | | | 7.18-7.06 (m, 2H), 7.03 (d, J = 8.4 Hz, 2H), 6.60 |
| | | | | (d, J = 8.0 Hz, 1H), 5.98 (s, 1H), 5.39 (dd, J = |
| | | | | 12.8, 5.4 Hz, 1H), 4.99-4.77 (m, 1H), 4.11 (t, J = |
| | | | | 8.3 Hz, 2H), 3.41 (s, 3H), 3.31-3.25 (m, 2H), |
| | | | | 3.24-3.16 (m, 4H), 3.10-2.99 (m, 3H), 2.95 (d, J = |
| | | | | 4.9 Hz, 3H), 2.93-2.85 (m, 1H), 2.80-2.71 (m, |
| | | | | 1H), 2.69-2.60 (m, 3H), 2.59-2.52 (m, 4H), 2.28 |
| | | | | (d, J = 7.1 Hz, 2H), 2.08-2.01 (m, 1H), 1.89- |
| | | | | 1.82 (m, 2H), 1.77-1.65 (m, 1H), 1.31-1.22 (m, |
| | | | | 3H), 1.04-0.90 (m, 1H) |
| K552 | KE | IS | 885.6 | 11.09 (s, 1H), 8.81 (d, J = 4.2 Hz, 1H), 8.62- |
| | | | | 8.57 (m, 2H), 7.90-7.76 (m, 3H), 7.74 (dd, J = |
| | | | | 7.9, 1.6 Hz, 1H), 7.53-7.44 (m, 2H), 7.23 (t, J = |
| | | | | 7.9 Hz, 1H), 7.06-6.88 (m, 3H), 6.13 (s, 1H), |
| | | | | 5.34 (dd, J = 12.7, 5.4 Hz, 1H), 4.84-4.60 (m, |
| | | | | 1H), 3.57 (s, 2H), 3.50 (s, 3H), 3.34 (s, 3H), |
| | | | | 3.34-3.32 (m, 6H), 2.97-2.80 (m, 7H), 2.77-2.57 |
| | | | | (m, 4H), 2.28-2.10 (m, 1H), 2.02-1.80 (m, 4H), |
| | | | | 1.62-1.42 (m, 4H), 1.13-1.00 (m, 1H), 0.73-0.58 |
| | | | | (m, 1H) |
| K553 | IT | IS | 885.5 | 11.08 (s, 1H), 8.81 (d, J = 4.2 Hz, 1H), 8.63- |
| | | | | 8.55 (m, 2H), 7.89-7.77 (m, 3H), 7.75 (d, J = 7.7 |
| | | | | Hz, 1H), 7.51-7.46 (m, 2H), 7.23 (t, J = 7.9 Hz, |
| | | | | 1H), 7.06 (s, 1H), 6.99 (d, J = 8.1 Hz, 1H), 6.89 |
| | | | | (d, J = 8.1 Hz, 1H), 6.13 (s, 1H), 5.33 (dd, J = |
| | | | | 12.7, 5.4 Hz, 1H), 4.82-4.64 (m, 1H), 3.58-3.53 |
| | | | | (m, 3H), 3.51 (s, 3H), 3.34-3.28 (m, 3H), 2.94- |
| | | | | 2.83 (m, 6H), 2.78-2.53 (m, 6H), 2.49-2.35 (m, |
| | | | | 4H), 2.05-1.80 (m, 5H), 1.58-1.47 (m, 2H), |
| | | | | 1.44-1.32 (m, 2H), 1.11-1.02 (m, 1H), 0.74-0.58 |
| | | | | (m, 1H) |
| K557 | AI | JE | 877.5 | 11.11 (s, 1H), 8.69-8.57 (m, 3H), 8.34-8.28 (m, |
| | | | | 1H), 7.97 (d, J = 8.1 Hz, 1H), 7.88-7.78 (m, 3H), |
| | | | | 7.60 (d, J = 8.0 Hz, 1H), 7.55 (d, J = 8.3 Hz, |
| | | | | 2H), 7.48-7.44 (m, 2H), 7.42 (d, J = 1.7 Hz, 1H), |
| | | | | 7.27 (dd, J = 8.3, 1.7 Hz, 1H), 7.14 (d, J = 8.2 |
| | | | | Hz, 1H), 7.01 (d, J = 8.4 Hz, 2H), 5.39 (dd, J = |
| | | | | 12.7, 5.4 Hz, 1H), 4.65-4.43 (m, 1H), 4.34 (t, J = |
| | | | | 4.2 Hz, 2H), 3.62 (s, 2H), 3.57 (t, J = 3.9 Hz, |
| | | | | 2H), 3.40 (s, 3H), 3.21 (t, J = 4.9 Hz, 4H), 2.97- |
| | | | | 2.87 (m, 1H), 2.79-2.69 (m, 1H), 2.68-2.61 (m, |
| | | | | 2H), 2.57 (t, J = 5.0 Hz, 4H), 2.08-2.01 (m, 1H), |
| | | | | 0.84-0.77 (m, 1H), 0.51-0.37 (m, 1H) |
| K563 | AI | IY | 867.5 | 11.11 (s, 1H), 8.92 (s, 1H), 8.77-8.67 (m, 2H), |
| | | | | 7.93-7.84 (m, 2H), 7.83-7.73 (m, 2H), 7.72-7.65 |
| | | | | (m, 1H), 7.61-7.51 (m, 3H), 7.45-7.39 (m, 1H), |
| | | | | 7.37-7.24 (m, 2H), 7.14 (d, J = 8.2 Hz, 1H), 7.02 |
| | | | | (d, J = 8.6 Hz, 2H), 5.98 (s, 1H), 5.39 (dd, J = |
| | | | | 12.7, 5.4 Hz, 1H), 4.78-4.56 (m, 1H), 3.66 (s, |
| | | | | 2H), 3.40 (s, 3H), 3.27-3.18 (m, 4H), 2.90 (d, J = |
| | | | | 5.0 Hz, 3H), 2.85-2.69 (m, 3H), 2.69-2.64 (m, |
| | | | | 1H), 2.64-2.58 (m, 4H), 2.08-2.01 (m, 1H), 1.06- |
| | | | | 0.92 (m, 1H), 0.59-0.44 (m, 1H) |
| K564 | AI | LI | 806.4 | 11.11 (s, 1H), 8.68-8.54 (m, 2H), 7.84 (s, 1H), |
| | | | | 7.62-7.50 (m, 4H), 7.42 (d, J = 1.7 Hz, 1H), |
| | | | | 7.40-7.35 (m, 2H), 7.28 (dd, J = 8.2, 1.7 Hz, |
| | | | | 1H), 7.14 (d, J = 8.2 Hz, 1H), 7.03 (d, J = 8.5 |
| | | | | Hz, 2H), 6.00 (s, 1H), 5.42-5.31 (m, 1H), 4.80- |
| | | | | 4.60 (m, 1H), 3.70 (s, 2H), 3.40 (s, 3H), 3.26- |
| | | | | 3.20 (m, 4H), 2.97-2.87 (m, 4H), 2.85-2.78 (m, |
| | | | | 1H), 2.77-2.71 (m, 1H), 2.70-2.60 (m, 5H), |
| | | | | 2.10-1.96 (m, 1H), 1.09-0.98 (m, 1H), 0.50-0.38 |
| | | | | (m, 1H) |
| K566 | AC | IN | 957.5 | 11.12 (s, 1H), 8.89 (d, J = 4.6 Hz, 1H), 8.58 (d, J = |
| | | | | 4.2 Hz, 1H), 7.96 (s, 1H), 7.83-7.65 (m, 4H), |
| | | | | 7.59-7.54 (m, 2H), 7.46-7.44 (m, 1H), 7.33-7.23 |
| | | | | (m, 5H), 7.17 (d, J = 8.0 Hz, 1H), 6.00-5.98 (m, |
| | | | | 1H), 5.40 (dd, J = 12.8, 5.4 Hz, 1H), 4.97-4.76 |
| | | | | (m, 1H), 4.13-4.08 (m, 2H), 3.62-3.40 (m, 7H), |
| | | | | 3.11-2.86 (m, 5H), 2.78-2.53 (m, 5H), 2.48-2.31 |
| | | | | (m, 6H), 2.08-2.03 (m, 1H), 1.94-1.83 (m, 4H), |
| | | | | 1.56-1.14 (m, 6H), 1.03-0.91 (m, 1H) |
| K567 | AC | IS | 961.5 | 11.12 (s, 1H), 8.81 (d, J = 4.2 Hz, 1H), 8.62 (s, |
| | | | | 1H), 8.57 (s, 1H), 7.91-7.77 (m, 3H), 7.75 (d, J = |
| | | | | 7.9 Hz, 1H), 7.61-7.57 (m, 2H), 7.52-7.42 (m, |
| | | | | 3H), 7.36-7.27 (m, 3H), 7.24 (t, J = 7.9 Hz, 1H), |
| | | | | 7.18 (d, J = 8.2 Hz, 1H), 6.14 (d, J = 1.7 Hz, |
| | | | | 1H), 5.40 (dd, J = 12.9, 5.4 Hz, 1H), 4.83-4.63 |
| | | | | (m, 1H), 3.72-3.55 (m, 3H), 3.51 (s, 3H), 3.41 |
| | | | | (s, 3H), 3.02-2.81 (m, 5H), 2.79-2.55 (m, 5H), |
| | | | | 2.48-2.34 (m, 3H), 2.08-2.02 (m, 1H), 1.98-1.86 |
| | | | | (m, 3H), 1.57-1.31 (m, 4H), 1.28-1.23 (m, 4H), |
| | | | | 1.11-1.01 (m, 1H), 0.72-0.60 (m, 1H) |
| K573 | FQ | JJ | 893.4 | 11.12 (s, 1H), 8.93 (d, J = 4.8 Hz, 1H), 7.94 (s, |
| | | | | 1H), 7.66 (q, J = 4.9 Hz, 1H), 7.52-7.48 (m, 2H), |
| | | | | 7.37 (s, 1H), 7.29-7.23 (m, 2H), 7.17-7.03 (m, |
| | | | | 2H), 6.57 (d, J = 8.1 Hz, 1H), 6.49 (d, J = 8.1 |
| | | | | Hz, 2H), 5.96 (s, 1H), 5.38 (dd, J = 12.8, 5.5 Hz, |
| | | | | 1H), 4.97-4.77 (m, 1H), 4.08 (t, J = 8.4 Hz, 2H), |
| | | | | 3.89 (s, 4H), 3.40 (s, 3H), 3.39-3.34 (m, 4H), |
| | | | | 3.27-3.21 (m, 2H), 3.07-3.00 (m, 3H), 2.97-2.87 |
| | | | | (m, 4H), 2.80-2.53 (m, 4H), 2.34-2.26 (m, 2H), |
| | | | | 2.08-2.02 (m, 1H), 1.79-1.72 (m, 2H), 1.44-1.13 |
| | | | | (m, 4H), 1.05-0.91 (m, 1H) |
| K574 | FQ | IN | 887.4 | 11.10 (s, 1H), 8.88 (d, J = 4.7 Hz, 1H), 8.59 (d, J = |
| | | | | 2.6 Hz, 1H), 7.96 (s, 1H), 7.83-7.64 (m, 4H), |
| | | | | 7.55-7.46 (m, 2H), 7.37 (d, J = 1.7 Hz, 1H), |
| | | | | 7.33-7.27 (m, 2H), 7.23 (d, J = 8.3 Hz, 1H), 7.12 |
| | | | | (d, J = 8.2 Hz, 1H), 6.56-6.44 (m, 2H), 6.02- |
| | | | | 5.98 (m, 1H), 5.38 (dd, J = 12.8, 5.4 Hz, 1H), |
| | | | | 4.98-4.78 (m, 1H), 4.17-4.11 (m, 2H), 3.97-3.85 |
| | | | | (m, 4H), 3.66-3.62 (m, 2H), 3.38-3.34 (m, 2H), |
| | | | | 3,32-3,27 (m, 7H), 3.08-3.01 (m, 1H), 2.99-2.86 |
| | | | | (m, 4H), 2.79-2,63 (m, 2H), 2.11-1.97 (m, 1H), |
| | | | | 1.28-1.17 (m, 1H), 1.03-0.93 (m, 1H) |
| K575 | AI | KO | 798.4 | 11.11 (s, 1H), 8.89 (d, J = 4.7 Hz, 1H), 7.95 (s, |
| | | | | 1H), 7.72-7.64 (m, 1H), 7.62-7.53 (m, 3H), 7.42 |
| | | | | (d, J = 1.7 Hz, 1H), 7.28 (dd, J = 8.2, 1.7 Hz, |
| | | | | 1H), 7.20-7.11 (m, 2H), 7.02 (d, J = 8.4 Hz, 2H), |
| | | | | 6.92 (d, J = 7.6 Hz, 1H), 6.01 (s, 1H), 5.39 (dd, J = |
| | | | | 12.8, 5.4 Hz, 1H), 4.98-4.79 (m, 1H), 4.16 (t, J = |
| | | | | 8.6 Hz, 2H), 3.53 (s, 2H), 3.40 (s, 3H), 3.28- |
| | | | | 3.16 (m, 6H), 3.09-3.00 (m, 1H), 2.96 (d, J = 4.9 |
| | | | | Hz, 3H), 2.94-2.86 (m, 1H), 2.80-2.70 (m, 1H), |
| | | | | 2.69-2.60 (m, 1H), 2.58 (t, J = 4.8 Hz, 4H), |
| | | | | 2.10-2.00 (m, 1H), 1.29-1.17 (m, 1H), 1.03-0.91 |
| | | | | (m, 1H) |
| K577 | AI | JD | 897.4 | 11.11 (s, 1H), 8.79-8.63 (m, 3H), 7.95-7.88 (m, |
| | | | | 3H), 7.79-7.72 (m, 1H), 7.60-7.52 (m, 3H), |
| | | | | 7.45-7.39 (m, 1H), 7.29-7.24 (m, 2H), 7.14 (d, J = |
| | | | | 8.3 Hz, 1H), 7.03 (d, J = 8.4 Hz, 2H), 6.23 (s, |
| | | | | 1H), 5.39 (dd, J = 12.8, 5.4 Hz, 1H), 4.84-4.65 |
| | | | | (m, 1H), 3.67 (s, 2H), 3.51 (s, 3H), 3.40 (s, 3H), |
| | | | | 3.26-3.20 (m, 4H), 2.95-2.86 (m, 5H), 2.79-2.73 |
| | | | | (m, 1H), 2.67-2.62 (m, 5H), 2.09-1.97 (m, 1H), |
| | | | | 1.13-1.08 (m, 1H), 0.80-0.64 (m, 1H) |
| K581 | DG | JM | 746.4 | 11.02 (s, 1H), 9.06 (s, 1H), 8.76 (d, J = 4.1 Hz, |
| | | | | 1H), 7.82 (s, 1H), 7.54-7.44 (m, 2H), 7.31 (t, J = |
| | | | | 7.8 Hz, 1H), 7.22 (d, J = 2.0 Hz, 1H), 6.98 (d, J = |
| | | | | 7.5 Hz, 1H), 6.88 (d, J = 8.6 Hz, 1H), 6.38 (d, |
| | | | | J = 2.2 Hz, 1H), 6.21 (dd, J = 8.6, 2.2 Hz, 1H), |
| | | | | 5.77 (s, 1H), 5.25 (dd, J = 12.8, 5.4 Hz, 1H), |
| | | | | 3.47 (s, 2H), 3.41-3.35 (m, 2H), 3.29 (s, 3H), |
| | | | | 3.25-3.19 (m, 1H), 2.96-2.75 (m, 8H), 2.73-2.56 |
| | | | | (m, 2H), 2.15-1.86 (m, 4H), 1.76-1.69 (m, 2H), |
| | | | | 1.66-1.54 (m, 1H), 1.35-1.22 (m, 4H), 0.75-0.70 |
| | | | | (m, 2H), 0.42-0.36 (m, 2H) |
| K583 | DE | JM | 865.5 | 11.11 (s, 1H), 9.10 (s, 1H), 8.73 (s, 1H), 7.83 (s, |
| | | | | 1H), 7.58-7.47 (m, 3H), 7.40-7.33 (m, 2H), |
| | | | | 7.26-7.19 (m, 2H), 7.14-7.06 (m, 2H), 6.63-6.59 |
| | | | | (2H), 6.51 (s, 1H), 5.76 (s, 1H), 5.38 (dd, J = |
| | | | | 12.7, 5.4 Hz, 1H), 3.54-3.50 (m, 2H), 3.40 (s, |
| | | | | 3H), 3,32-3.28 (m, 4H), 3.09-3.02 (m, 1H), |
| | | | | 2.96-2.80 (m, 4H), 2.80-2.57 (m, 4H), 2.54-2.45 |
| | | | | (m, 8H), 2.24-2.14 (m, 2H), 2.08-2.01 (m, 1H), |
| | | | | 1.99-1.90 (m, 2H), 1.83-1.63 (m, 3H), 0.75-0.70 |
| | | | | (m, 2H), 0.37-0.32 (m, 2H) |
| K586 | GY | JM | 795.4 | 11.11 (s, 1H), 9.10 (s, 1H), 8.74 (d, J = 3.9 Hz, |
| | | | | 1H), 8.45 (d, J = 2.6 Hz, 1H), 7.87-7.83 (m, 2H), |
| | | | | 7.51-7.46 (m, 2H), 7.45-7.43 (m, 1H), 7.39-7.31 |
| | | | | (m, 2H), 7.30-7.26 (m, 1H), 7.15 (d, J = 8.2 Hz, |
| | | | | 1H), 7.06-7.00 (m, 1H), 6.93 (d, J = 8.9 Hz, 1H), |
| | | | | 5.77 (s, 1H), 5.39 (dd, J = 12.9, 5.4 Hz, 1H), |
| | | | | 3.89 (s, 2H), 3.57-3.50 (m, 4H), 3.40 (s, 3H), |
| | | | | 3.32 (s, 4H), 2.98-2.91 (m, 1H), 2.88 (d, J = 4.9 |
| | | | | Hz, 3H), 2.81-2.71 (m, 1H), 2.70-2.58 (m, 2H), |
| | | | | 2.09-2.01 (m, 1H), 1.82-1.75 (m, 4H), 0.76-0.70 |
| | | | | (m, 2H), 0.42-0.36 (m, 2H) |
| K587 | KW | JM | 810.4 | 11.12 (s, 1H), 9.07 (s, 1H), 8.75 (d, J = 4.1 Hz, |
| | | | | 1H), 7.82 (s, 1H), 7.61-7.57 (m, 2H), 7.50-7.44 |
| | | | | (m, 3H), 7.35-7.27 (m, 4H), 7.24-7.15 (m, 2H), |
| | | | | 6.96 (d, J = 7.5 Hz, 1H), 5.76 (s, 1H), 5.40 (dd, J = |
| | | | | 12.8, 5.4 Hz, 1H), 3.48 (s, 2H), 3.41 (s, 3H), |
| | | | | 2.98-2.78 (m, 8H), 2.76-2.70 (m, 1H), 2.70-2.58 |
| | | | | (m, 3H), 2.50 (s, 3H), 2.38-2.28 (m, 4H), 2.11- |
| | | | | 1.99 (m, 1H), 0.76-0.70 (m, 2H), 0.41-0.34 (m, 2H) |
| K589 | AD | JM | 816.5 | 11.05 (s, 1H), 9.09 (s, 1H), 8.73 (d, J = 4.0 Hz, |
| | | | | 1H), 7.83 (s, 1H), 7.50-7.43 (m, 2H), 7.40-7.27 |
| | | | | (m, 2H), 7.03 (s, 1H), 6.95 (d, J = 8.6 Hz, 1H), |
| | | | | 6.85 (d, J = 2.2 Hz, 1H), 6.65 (dd, J = 8.6, 2.3 |
| | | | | Hz, 1H), 5.76 (s, 1H), 5.29 (dd, J = 12.9, 5.4 Hz, |
| | | | | 1H), 4.11 (s, 2H), 3.95 (s, 2H), 3.73-3.50 (m, |
| | | | | 2H), 3.31 (s, 3H), 3.23-2.99 (m, 7H), 2.96-2.81 |
| | | | | (m, 4H), 2.75-2.57 (m, 3H), 2.54-2.47 (m, 5H), |
| | | | | 2.28-2.08 (m, 2H), 2.07-1.81 (m, 3H), 1.44-1.33 |
| | | | | (m, 2H), 0.75-0.70 (m, 2H), 0.39-0.33 (m, 2H) |
| K604 | BO | JM | 792.4 | 11.12 (s, 1H), 9.07 (s, 1H), 8.75 (d, J = 4.0 Hz, |
| | | | | 1H), 8.51 (s, 2H), 8.31 (s, 1H), 7.82 (s, 1H), |
| | | | | 7.50-7.43 (m, 2H), 7.40-7.38 (m, 1H), 7.33-7.28 |
| | | | | (m, 1H), 7.26-7.22 (m, 1H), 7.20-7.16 (m, 2H), |
| | | | | 6.96-6.93 (m, 1H), 5.76 (s, 1H), 5.40 (dd, J = |
| | | | | 12.7, 5.4 Hz, 1H), 4.18 (s, 4H), 3.57 (s, 2H), |
| | | | | 3.37-3.36 (m, 7H), 2.93-2.89 (m, 1H), 2.88 (d, J = |
| | | | | 4.9 Hz, 3H), 2.86-2.81 (m, 1H), 2.78-2.59 (m, |
| | | | | 2H), 2.10-2.00 (m, 1H), 1.26-1.23 (m, 1H), |
| | | | | 0.76-0.69 (m, 2H), 0.41-0.37 (m, 2H) |
| K608 | AV | JM | 820.6 | 11.12 (s, 1H), 9.05 (s, 1H), 8.75 (d, J = 4.0 Hz, |
| | | | | 1H), 8.45 (s, 2H), 8.14 (s, 1H), 7.81 (s, 1H), |
| | | | | 7.50-7.42 (m, 2H), 7.40-7.38 (m, 1H), 7.35-7.29 |
| | | | | (m, 1H), 7.28-7.26 (m, 1H), 7.25-7.22 (m, 1H), |
| | | | | 7.20-7.16 (m, 1H), 7.01 (d, J = 7.5 Hz, 1H), 5.76 |
| | | | | (s, 1H), 5.41 (dd, J = 12.7, 5.4 Hz, 1H), 3.75 (s, |
| | | | | 4H), 3.53 (s, 2H), 3.37 (s, 3H), 2.95-2.90 (m, |
| | | | | 1H), 2.88 (d, J = 4.9 Hz, 3H), 2.84-2.76 (m, 1H), |
| | | | | 2.75-2.60 (m, 2H), 2.49-2.26 (m, 5H), 2.10-2.01 |
| | | | | (m, 1H), 1.71-1.62 (m, 2H), 1.61-1.51 (m, 2H), |
| | | | | 0.72-0.65 (m, 2H), 0.38-0.31 (m, 2H) |
| K609 | LH | IN | 943.5 | 11.11 (s, 1H), 8.89 (d, J = 4.7 Hz, 1H), 8.63 (s, |
| | | | | 1H), 7.97 (s, 1H), 7.86-7.67 (m, 4H), 7.56-7.51 |
| | | | | (m, 2H), 7.43-7.39 (m, 1H), 7.35-7.25 (m, 3H), |
| | | | | 7.15-7.10 (m, 1H), 7.02-6.97 (m, 2H), 6.02 (s, |
| | | | | 1H), 5.41-5.35 (m, 1H), 4.99-4.80 (m, 2H), |
| | | | | 4.17-4.10 (m, 2H), 3.62-3.56 (m, 1H), 3.47-3.42 |
| | | | | (m, 5H), 3.20-3.11 (m, 4H), 3.07-3.01 (m, 1H), |
| | | | | 2.99-2.86 (m, 4H), 2.81-2.59 (m, 2H), 2,52-2.40 |
| | | | | (m, 4H), 2.11-1.98 (m, 1H), 1.67-1.45 (m, 8H), |
| | | | | 1.27-1.17 (m, 1H), 1.04-0.93 (m, 1H) |
| K611 | AI | LJ | 899.5 | 11.09 (s, 1H), 8.73-8.62 (m, 2H), 8.48 (s, 1H), |
| | | | | 7.91 (dd, J = 8.1, 2.2 Hz, 1H), 7.83 (s, 1H), |
| | | | | 7.70-7.64 (m, 2H), 7.62-7.55 (m, 3H), 7.52-7.47 |
| | | | | (m, 1H), 7.44-7.40 (m, 2H), 7.30-7.27 (m, 1H), |
| | | | | 7.20-7.13 (m, 1H), 7.07-7.92 (m, 2H), 6.02 (s, |
| | | | | 1H), 5.39 (dd, J = 12.8, 5.4 Hz, 1H), 4.77-4.56 |
| | | | | (m, 1H), 3.67 (s, 2H), 3.40 (s, 3H), 3.25-3.21 |
| | | | | (m, 4H), 2.97-2.87 (m, 4H), 2.82-2.72 (m, 2H), |
| | | | | 2.68-2.60 (m, 6H), 2.09-2.01 (m, 1H), 1.05-0.96 |
| | | | | (m, 1H), 0.43-0.32 (m, 1H) |
| K613 | FD | JM | 706.4 | 11.08 (s, 1H), 9.08 (s, 1H), 8.77 (d, J = 4.0 Hz, |
| | | | | 1H), 8.36-8.29 (m, 2H), 7.85-7.80 (m, 1H), |
| | | | | 7.48-7.42 (m, 2H), 7.31 (t, J = 7.8 Hz, 1H), |
| | | | | 7.23-7.20 (m, 1H), 7.06 (d, J = 1.5 Hz, 1H), |
| | | | | 7.01-6.95 (m, 2H), 6.89 (dd, J = 8.0, 1.5 Hz, |
| | | | | 1H), 5.77 (s, 1H), 5.32 (dd, J = 12.8, 5.4 Hz, |
| | | | | 1H), 3.31 (s, 3H), 2.90-2.80 (m, 5H), 2.78-2.72 |
| | | | | (m, 2H), 2.72-2.57 (m, 2H), 2.45-2.34 (m, 12H), |
| | | | | 2.02-1.92 (m, 1H), 0.75-0.68 (m, 2H), 0.42-0.34 |
| | | | | (m, 2H) |
| K619 | AI | JF | 885.5 | 11.11 (s, 1H), 8.80 (d, J = 4.2 Hz, 1H), 8.34 (s, |
| | | | | 1H), 7.84 (s, 1H), 7.56 (d, J = 8.5 Hz, 2H), 7.47- |
| | | | | 7.39 (m, 2H), 7.35-7.25 (m, 2H), 7.14 (d, J = 8.3 |
| | | | | Hz, 1H), 7.08-6.96 (m, 3H), 6.74 (d, J = 8.0 Hz, |
| | | | | 1H), 6.11 (s, 1H), 5.39 (dd, J = 12.8, 5.4 Hz, |
| | | | | 1H), 4.85-4.65 (m, 1H), 3.79 (s, 3H), 3.50 (t, J = |
| | | | | 10.9 Hz, 2H), 3.41 (s, 3H), 3.27-3.14 (m, 4H), |
| | | | | 2.99-2.81 (m, 5H), 2.81-2.70 (m, 1H), 2.68-2.60 |
| | | | | (m, 3H), 2.59-2.53 (m, 4H), 2.28 (d, J = 7.1 Hz, |
| | | | | 2H), 2.10-2.01 (m, 1H), 1.92-1.84 (m, 2H), |
| | | | | 1.76-1.65 (m, 1H), 1.40-1.28 (m, 2H), 1.16-1.04 |
| | | | | (m, 1H), 0.77-0.62 (m, 1H) |
| K621 | AI | LE | 884.4 | 11.12 (s, 1H), 8.80 (d, J = 4.2 Hz, 1H), 8.41 (s, |
| | | | | 1H), 7.84 (s, 1H), 7.56 (d, J = 8.4 Hz, 2H), 7.50- |
| | | | | 7.40 (m, 3H), 7.31-7.25 (m, 1H), 7.17-6.98 (m, |
| | | | | 5H), 6.07 (s, 1H), 5.39 (dd, J = 12.8, 5.4 Hz, |
| | | | | 1H), 4.85-4.61 (m, 1H), 3.67 (s, 3H), 3.41 (s, |
| | | | | 3H), 3.23-3.16 (m, 4H), 2.98-2.79 (m, 5H), |
| | | | | 2.79-2.53 (m, 3H), 2.53-2.47 (m, 4H), 2.25-2.19 |
| | | | | (m, 2H), 2.09-2.00 (m, 1H), 1.99-1.88 (m, 2H), |
| | | | | 1.85-1.75 (m, 2H), 1.68-1.45 (m, 4H), 1.12-1.02 |
| | | | | (m, 2H), 0.63-0.50 (m, 1H) |
| K622 | JP | JF | 884.5 | 11.13 (s, 1H), 8.80 (d, J = 4.2 Hz, 1H), 8.34 (s, |
| | | | | 1H), 7.85 (s, 1H), 7.65 (d, J = 7.7 Hz, 2H), 7.49 |
| | | | | (s, 1H), 7.44 (t, J = 4.9 Hz, 1H), 7.37-7.31 (m, |
| | | | | 4H), 7.19 (d, J = 8.2 Hz, 1H), 7.02 (t, J = 8.0 Hz, |
| | | | | 1H), 6.75 (d, J = 8.1 Hz, 1H), 6.11 (s, 1H), 5.41 |
| | | | | (dd, J = 12.8, 5.4 Hz, 1H), 4.86-4.66 (m, 1H), |
| | | | | 3.80 (s, 3H), 3.51 (t, J = 11.0 Hz, 2H), 3.42 (s, |
| | | | | 3H), 3,36-3.30 (m, 8H), 2.95-2.83 (m, 5H), |
| | | | | 2.69-2.59 (m, 3H), 2.11-2.03 (m, 1H), 1.97-1.68 |
| | | | | (m, 7H), 1.46-1.33 (m, 2H), 1.16-1.08 (m, 1H), |
| | | | | 0.76-0.64 (m, 1H) |
| K623 | FQ | JF | 897.4 | 11.11 (s, 1H), 8.79 (d, J = 4.2 Hz, 1H), 8.33 (s, |
| | | | | 1H), 7.84 (s, 1H), 7.54-7.48 (m, 2H), 7.47-7.34 |
| | | | | (m, 2H), 7.30 (d, J = 7.9 Hz, 1H), 7.27-7.20 (m, |
| | | | | 1H), 7.13 (d, J = 8.2 Hz, 1H), 7.08-6.96 (m, 1H), |
| | | | | 6.73 (dd, J = 8.2, 1.5 Hz, 1H), 6.54-6.48 (m, |
| | | | | 2H), 6.10 (s, 1H), 5.38 (dd, J = 12.8, 5.4 Hz, |
| | | | | 1H), 4.87-4.67 (m, 1H), 3.91 (s, 3H), 3.78 (s, |
| | | | | 3H), 3.50-3.43 (m, 2H), 3.45-3.38 (s, 3H), 3.31- |
| | | | | 3.26 (m, 6H), 2.97-2.81 (m, 4H), 2.80-2.70 (m, |
| | | | | 1H), 2.68-2.55 (m, 3H), 2.35-2.30 (m, 2H), |
| | | | | 2.08-2.00 (m, 1H), 1.84-1.75 (m, 2H), 1.45-1.26 |
| | | | | (m, 3H), 1.14-1.04 (m, 1H), 0.76-0.61 (m, 1H) |
| K624 | AI | KG | 897.5 | 11.11 (s, 1H), 8.69-8.58 (m, 3H), 7.89-7.79 (m, |
| | | | | 3H), 7.75 (dd, J = 8.9, 6.5 Hz, 1H), 7.59-7.50 |
| | | | | (m, 3H), 7.42 (d, J = 1.7 Hz, 1H), 7.31-7.19 (m, |
| | | | | 2H), 7.14 (d, J = 8.2 Hz, 1H), 7.05-6.99 (m, 2H), |
| | | | | 5.90 (s, 1H), 5.39 (dd, J = 12.8, 5.4 Hz, 1H), |
| | | | | 4.68-4.48 (m, 1H), 3.65 (s, 2H), 3.56 (s, 3H), |
| | | | | 3.40 (s, 3H), 3.27-3.17 (m, 4H), 2.97-2.86 (m, |
| | | | | 4H), 2.80-2.70 (m, 2H), 2.69-2.64 (m, 1H), |
| | | | | 2.64-2.57 (m, 4H), 2.08-2.01 (m, 1H), 0.96-0.83 |
| | | | | (m, 1H), 0.31-0.19 (m, 1H) |
| K625 | LJ | IN | 826.5 | 10.42 (s, 1H), 8.88 (t, J = 3.2 Hz, 1H), 8.58-8.56 |
| | | | | (m, 1H), 7.96 (s, 1H), 7.81-7.67 (m, 4H), 7.47- |
| | | | | 7.34 (m, 3H), 7.33-7.25 (m, 2H), 7.24-7.21 (m, |
| | | | | 1H), 5.99 (s, 1H), 4.98-4.78 (m, 1H), 4.16-4.09 |
| | | | | (m, 2H), 3.83 (t, J = 6.6 Hz, 2H), 3.65-3.49 (m, |
| | | | | 4H), 3.46-3.35 (m, 5H), 3.08-3.02 (m, 1H), 2.96 |
| | | | | (t, J = 4.1 Hz, 3H), 2.72 (t, J = 6.5 Hz, 2H), |
| | | | | 2.43-2.34 (m, 4H), 1.56-1.35 (m, 8H), 1.27-1.20 |
| | | | | (m, 1H), 1.03-0.89 (m, 1H) |
| K626 | LL | HT | 986.5 | 11.11 (s, 1H), 8.88 (d, J = 4.7 Hz, 1H), 8.73 (d, J = |
| | | | | 2.2 Hz, 1H), 7.99-7.92 (m, 2H), 7.85 (d, J = |
| | | | | 8.1 Hz, 1H), 7.80 (d, J = 7.6 Hz, 1H), 7.75-7.67 |
| | | | | (m, 1H), 7.54 (d, J = 8.6 Hz, 2H), 7.41 (d, J = |
| | | | | 1.7 Hz, 1H), 7.38-7.34 (m, 1H), 7.34-7.29 (m, |
| | | | | 1H), 7.27 (dd, J = 8.2, 1.7 Hz, 1H), 7.14 (d, J = |
| | | | | 8.3 Hz, 1H), 7.01 (d, J = 8.6 Hz, 2H), 6.03 (s, |
| | | | | 1H), 5.39 (dd, J = 12.8, 5.4 Hz, 1H), 5.00-4.76 |
| | | | | (m, 1H), 4.17 (t, J = 8.5 Hz, 2H), 3.84-3.61 (m, |
| | | | | 4H), 3.48 (t, J = 8.5 Hz, 2H), 3.40 (s, 3H), 3.09- |
| | | | | 3.01 (m, 1H), 2.98 (d, J = 4.9 Hz, 3H), 2.95-2.86 |
| | | | | (m, 1H), 2.78-2.60 (m, 4H), 2.57-2.52 (m, 2H), |
| | | | | 2.47-2.47 (m, 4H), 2.24 (d, J = 7.1 Hz, 2H), |
| | | | | 2.08-2.01 (m, 1H), 1.87-1.79 (m, 2H), 1.76-1.65 |
| | | | | (m, 1H), 1.32-1.16 (m, 3H), 1.05-0.91 (m, 1H) |
| K628 | JB | HT | 990.5 | 11.10 (s, 1H), 8.80 (d, J = 4.3 Hz, 1H), 8.75 (t, J = |
| | | | | 1.6 Hz, 1H), 8.61 (s, 1H), 7.98-7.90 (m, 2H), |
| | | | | 7.86 (s, 1H), 7.78 (dd, J = 8.0, 1.6 Hz, 1H), |
| | | | | 7.59-7.45 (m, 4H), 7.41 (d, J = 1.6 Hz, 1H), |
| | | | | 7.31-7.22 (m, 2H), 7.14 (d, J = 8.3 Hz, 1H), 7.01 |
| | | | | (d, J = 8.8 Hz, 2H), 6.13 (s, 1H), 5.39 (dd, J = |
| | | | | 12.7, 5.4 Hz, 1H), 4.83-4.59 (m, 1H), 3.84-3.61 |
| | | | | (m, 4H), 3.54 (s, 3H), 3.45-3.41 (m, 1H), 3.40 |
| | | | | (s, 3H), 3.36-3.33 (m, 1H), 2.98-2.81 (m, 5H), |
| | | | | 2.81-2.60 (m, 4H), 2.49-2.35 (m, 4H), 2.27-2.21 |
| | | | | (m, 2H), 2.11-2.01 (m, 1H), 1.87-1.79 (m, 2H), |
| | | | | 1.728-1.64(m, 1H), 1.30-1.19 (m, 2H), 1.13- |
| | | | | 1.00 (m, 1H), 0.71-0.56 (m, 1H) |
| K630 | AI | LM | 887.4 | 11.10 (s, 1H), 8.69-8.62 (m, 1H), 8.40-8.25 (m, |
| | | | | 2H), 8.20-8.15 (m, 1H), 7.89-7.75 (m, 2H), |
| | | | | 7.80-7.72 (m, 1H), 7.58-7.49 (m, 2H), 7.44-7.41 |
| | | | | (m, 2H), 7.31-7.24 (m, 3H), 7.16-7.10 (m, 1H), |
| | | | | 7.03-6.95 (m, 2H), 6.19-6.10 (m, 1H), 5.41-5.37 |
| | | | | (m, 1H), 4.89-4.78 (m, 1H), 3.68-3.60 (m, 2H), |
| | | | | 3.55-3.49 (m, 3H), 3.42-3.38 (m, 3H), 3.26-3.15 |
| | | | | (m, 4H), 2.94-2.87 (m, 4H), 2.79-2.66 (m, 2H), |
| | | | | 2.63-2.58 (m, 4H), 2.05 (m, 1H), 1.53 (d, J = 6.7 |
| | | | | Hz, 6H) |
| K631 | AI | LN | 883.4 | 11.12 (s, 1H), 8.66-8.57 (m, 2H), 8.14-8.10 (m, |
| | | | | 1H), 7.88-7.80 (m, 2H), 7.76-7.70 (m, 1H), |
| | | | | 7.57-7.50 (m, 2H), 7.48-7.44 (m, 1H), 7.43-7.38 |
| | | | | (m, 1H), 7.33-7.21 (m, 3H), 7.14 (d, J = 8.3 Hz, |
| | | | | 1H), 7.03-6.93 (m, 2H), 5.86 (s, 1H), 5.39 (dd, J = |
| | | | | 12.8, 5.4 Hz, 1H), 5.08-4.89 (m, 1H), 4.24- |
| | | | | 4.12 (m, 2H), 3.65-3.58 (m, 1H), 3.52-3.44 (m, |
| | | | | 1H), 3.40 (s, 3H), 3.25-3.16 (m, 4H), 3.01-2.87 |
| | | | | (m, 4H), 2.82-2.65 (m, 2H), 2.64-2.56 (m, 5H), |
| | | | | 2.55 (s, 1H), 2.08-2.01 (m, 1H), 1.61-1.56 (m, 6H) |
| K634 | KL | KO | 819.5 | 11.07 (s, 1H), 8.88 (d, J = 4.7 Hz, 1H), 7.95 (s, |
| | | | | 1H), 7.73-7.50 (m, 2H), 7.14 (t, J = 7.8 Hz, |
| | | | | 1H), 6.98-6.80 (m, 3H), 6.62 (d, J = 8.6 Hz, 1H), |
| | | | | 5.99 (s, 1H), 5.29 (dd, J = 12.7, 5.4 Hz, 1H), |
| | | | | 5.04-4.75 (m, 1H), 4.14 (t, J = 8.6 Hz, 2H), 3.47 |
| | | | | (s, 3H), 3.35-3.33 (m, 2H), 3.31 (s, 3H), 3.25- |
| | | | | 3.15 (m, 2H), 3.12-3.02 (m, 5H), 2.95 (d, J = 4.8 |
| | | | | Hz, 3H), 2.91-2.81 (m, 3H), 2.76-2.56 (m, 2H), |
| | | | | 2.25-2.15 (m, 2H), 2.11-1.92 (m, 3H), 1.78-1.67 |
| | | | | (m, 2H), 1.64-1.53 (m, 1H), 1.31-1.10 (m, 4H), |
| | | | | 1.08-0.97 (m, 1H) |
| K636 | T | IN | 799.5 | 11.06 (s, 1H), 8.89 (d, J = 4.7 Hz, 1H), 8.65 (d, J = |
| | | | | 2.2 Hz, 1H), 7.96 (s, 1H), 7.86 (dd, J = 8.2, |
| | | | | 2.2 Hz, 1H), 7.79-7.74 (m, 2H), 7.73-7.66 (m, |
| | | | | 1H), 7.37-7.32 (m, 1H), 7.31-7.27 (m, 1H), 6.95 |
| | | | | (d, J = 8.6 Hz, 1H), 6.85 (d, J = 2.2 Hz, 1H), |
| | | | | 6.64 (dd, J = 8.7, 2.3 Hz, 1H), 6.01 (s, 1H), 5.30 |
| | | | | (dd, J = 12.9, 5.3 Hz, 1H), 5.01-4.75 (m, 1H), |
| | | | | 4.15 (t, J = 8.5 Hz, 2H), 3.65 (s, 2H), 3.45 (t, J = |
| | | | | 8.5 Hz, 2H), 3.31 (s, 3H), 3.17-3.09 (m, 4H), |
| | | | | 3.08-3.01 (m, 1H), 2.97 (d, J = 4.9 Hz, 3H), |
| | | | | 2.95-2.83 (m, 1H), 2.75-2.63 (m, 2H), 2.62-2.57 |
| | | | | (m, 4H), 2.03-1.94 (m, 1H), 1.29-1.19 (m, 1H), |
| | | | | 1.05-0.92 (m, 1H) |
| K637 | BW | IN | 799.4 | 11.08 (s, 1H), 8.89 (d, J = 4.7 Hz, 1H), 8.69- |
| | | | | 8.63 (m, 1H), 7.96 (s, 1H), 7.90-7.85 (m, 1H), |
| | | | | 7.76 (d, J = 7.9 Hz, 2H), 7.73-7.67 (m, 1H), |
| | | | | 7.37-7.27 (m, 2H), 7.02-6.94 (m, 2H), 6.93-6.87 |
| | | | | (m, 1H), 6.02 (s, 1H), 5.36 (dd, J = 12.6, 5.4 Hz, |
| | | | | 1H), 5.00-4.75 (m, 1H), 4.16 (t, J = 8.5 Hz, 2H), |
| | | | | 3.68-3.64 (m, 5H), 3.46 (t, J = 8.5 Hz, 2H), |
| | | | | 3.08-3.02 (m, 2H), 2.97 (d, J = 4.9 Hz, 3H), |
| | | | | 2.94-2.86 (m, 2H), 2.77-2.58 (m, 2H), 2.04-1.96 |
| | | | | (m, 1H), 1.28-1.18 (m, 4H), 1.06-0.99 (m, 1H), |
| | | | | 0.98-0.92 (m, 1H), 0.89-0.79 (m, 2H) |
| K638 | T | JJ | 805.5 | 11.06 (s, 1H), 8.93 (d, J = 4.8 Hz, 1H), 7.94 (s, |
| | | | | 1H), 7.70-7.63 (m, 1H), 7.29 (d, J = 7.9 Hz, 1H), |
| | | | | 7.10 (t, J = 8.0 Hz, 1H), 6.95 (d, J = 8.5 Hz, 1H), |
| | | | | 6.85 (d, J = 2.2 Hz, 1H), 6.68-6.56 (m, 2H), 5.99 |
| | | | | (s, 1H), 5.30 (dd, J = 12.8, 5.4 Hz, 1H), 4.49- |
| | | | | 4.76 (m, 1H), 4.11 (t, J = 8.3 Hz, 2H), 3.34 (s, |
| | | | | 3H), 3.39-3.24 (m, 2H), 3.17-3.09 (m, 4H), |
| | | | | 3.08-3.02 (m, 2H), 2.95 (d, J = 4.9 Hz, 3H), |
| | | | | 2.91-2.84 (m, 1H), 2.76-2.61 (m, 4H), 2.60-2.58 |
| | | | | (m, 5H), 2.28 (d, J = 7.2 Hz, 2H), 2.03-1.96 (m, |
| | | | | 1H), 1.90-1.81 (m, 2H), 1.76-1.67 (m, 1H), |
| | | | | 1.29-1.20 (m, 3H), 1.04-0.92 (m, 1H) |
| K642 | AI | JH | 911.5 | 11.11 (s, 1H), 8.69-8.64 (m, 1H), 8.48 (d, J = 4.3 |
| | | | | Hz, 1H), 8.20 (s, 1H), 7.92-7.84 (m, 2H), 7.81 |
| | | | | (dd, J = 8.0, 1.7 Hz, 1H), 7.67-7.61 (m, 1H), |
| | | | | 7.60-7.54 (m, 2H), 7.53-7.46 (m, 2H), 7.44-7.40 |
| | | | | (m, 2H), 7.28 (dd, J = 8.2, 1.7 Hz, 1H), 7.14 (d, |
| | | | | J = 8.3 Hz, 1H), 7.06-6.99 (m, 2H), 5.39 (dd, J = |
| | | | | 12.8, 5.4 Hz, 1H), 4.80-4.57 (m, 1H), 4.40-4.33 |
| | | | | (m, 2H), 3.40 (s, 3H), 3.66 (s, 2H), 3.62-3.55 |
| | | | | (m, 2H), 3.29-3.18 (m, 4H), 2.99-2.70 (m, 3H), |
| | | | | 2.69-2.65 (m, 1H), 2.64-2.57 (m, 4H), 2.08-2.01 |
| | | | | (m, 1H), 1.07-0.95 (m, 1H), 0.52-0.40 (m, 1H) |
| K645 | KH | KXc | 791.4 | 11.05 (s, 1H), 8.95 (d, J = 4.7 Hz, 1H), 7.94 (s, |
| | | | | 1H), 7.66-7.51 (m, 1H), 7.29 (d, J = 8.0 Hz, 1H), |
| | | | | 7.11 (t, J = 8.0 Hz, 1H), 6.94 (d, J = 8.6 Hz, 1H), |
| | | | | 6.84 (d, J = 2.2 Hz, 1H), 6.66 (dd, J = 8.7, 2.2 |
| | | | | Hz, 1H), 6.58 (d, J = 8.1 Hz, 1H), 5.97 (s, 1H), |
| | | | | 5.26 (dd, J = 12.7, 5.4 Hz, 1H), 4.95-4.75 (m, |
| | | | | 1H), 4.09 (t, J = 8.3 Hz, 2H), 3.39-3.35 (m, 2H), |
| | | | | 3.30 (s, 3H), 3.09-2.95 (m, 7H), 2.94 (d, J = 4.9 |
| | | | | Hz, 3H), 2.91-2.83 (m, 1H), 2.52-2.59 (m, 8H), |
| | | | | 2.43-2.30 (m, 1H), 2.06-1.85 (m, 3H), 1.65-1.55 |
| | | | | (m, 2H), 1.27-1.14 (m, 1H), 1.01-0.91 (m, 1H) |
| K656 | KQ | KP | 882.4 | 11.08 (s, 1H), 8.90 (d, J = 4.8 Hz, 1H), 8.28 (d, J = |
| | | | | 5.9 Hz, 1H), 7.96 (s, 1H), 7.76-7.65 (m, 1H), |
| | | | | 7.26 (d, J = 4.4 Hz, 2H), 7.08 (s, 1H), 6.98 (t, J = |
| | | | | 7.9 Hz, 1H), 6.94-6.80 (m, 3H), 6.01 (s, 1H), |
| | | | | 5.35 (dd, J = 12.6, 5.4 Hz, 1H), 5.00-4.79 (m, |
| | | | | 1H), 4.13 (t, J = 8.5 Hz, 2H), 3.64 (s, 3H), 3.43- |
| | | | | 3.38 (m, 8H), 3.18-3.10 (m, 2H), 3.08-3.02 (m, |
| | | | | 1H), 2.97 (d, J = 4.9 Hz, 3H), 2.93-2.85 (m, 1H), |
| | | | | 2.75-2.61 (m, 5H), 2.30-2.24 (m, 2H), 2.04-. 96 |
| | | | | (m, 1H), 1.90-1.81 (m, 2H), 1.75-1.67 (m, 1H), |
| | | | | 1.42-1.32 (m, 2H), 1.28-1.18 (m, 2H), 1.05-1.05 |
| | | | | (m, 1H) |
| K657 | T | IZ | 799.5 | 11.05 (s, 1H), 8.89 (d, J = 4.7 Hz, 1H), 8.65 (d, J = |
| | | | | 5.0 Hz, 1H), 7.96 (s, 1H), 7.80-7.73 (m, 1H), |
| | | | | 7.73-7.66 (m, 2H), 7.42-7.35 (m, 1H), 7.35-7.26 |
| | | | | (m, 2H), 6.98-6.92 (m, 1H), 6.88-6.83 (m, 1H), |
| | | | | 6.68-6.61 (m, 1H), 6.01 (s, 1H), 5.29 (dd, J = |
| | | | | 12.8, 5.3 Hz, 1H), 5.00-4.76 (m, 1H), 4.48-4.40 |
| | | | | (m, 1H), 4.20-4.11 (m, 2H), 3.69 (s, 2H), 3.49- |
| | | | | 3.37 (m, 3H), 3.19-3.12 (m, 4H), 3.09-3.01 (m, |
| | | | | 1H), 2.97 (d, J = 4.9 Hz, 3H), 2.95-2.83 (m, 1H), |
| | | | | 2.74-2.58 (m, 7H), 2.03-1.95 (m, 1H), 1.29-1.15 |
| | | | | (m, 1H), 0.98 (m, 1H) |
| K658 | AI | IZ | 875.5 | 11.09 (s, 1H), 8.89 (d, J = 4.7 Hz, 1H), 8.69- |
| | | | | 8.63 (m, 1H), 7.96 (s, 1H), 7.77 (dd, J = 6.7, 2.3 |
| | | | | Hz, 1H), 7.74-7.66 (m, 2H), 7.59-7.53 (m, 2H), |
| | | | | 7.44-7.39 (m, 1H), 7.42-7.35 (m, 1H), 7.35-7.30 |
| | | | | (m, 1H), 7.34-7.24 (m, 2H), 7.14 (d, J = 8.3 Hz, |
| | | | | 1H), 7.02 (d, J = 8.8 Hz, 2H), 6.01 (s, 1H), 5.39 |
| | | | | (dd, J = 12.8, 5.4 Hz, 1H), 5.00-4.74 (m, 1H), |
| | | | | 4.48-4.40 (m, 1H), 4.16 (t, J = 8.5 Hz, 2H), 3.69 |
| | | | | (s, 2H), 3.49-3.37 (m, 3H), 3.28-3.21 (m, 4H), |
| | | | | 3.09-3.01 (m, 1H), 2.97 (d, J = 4.9 Hz, 3H), |
| | | | | 2.95-2.86 (m, 1H), 2.81-2.66 (m, 1H), 2.69-2.58 |
| | | | | (m, 5H), 2.55 (s, 1H), 2.09-2.01 (m, 1H), 1.30- |
| | | | | 1.16 (m, 1H), 1.05-0.90 (m, 1H) |
| K659 | LO | KXc | 795.4 | 11.06 (s, 1H), 8.60 (d, J = 4.2 Hz, 1H), 8.42 (s, |
| | | | | 1H), 7.98-7.90 (m, 2H), 7.87 (s, 1H), 7.56-7.50 |
| | | | | (m, 1H), 7.04-7.00 (m, 1H), 6.95 (d, J = 8.6 Hz, |
| | | | | 1H), 6.89-6.84 (m, 1H), 6.70-6.64 (m, 1H), 6.01 |
| | | | | (s, 1H), 5.30 (dd, J = 12.9, 5.3 Hz, 1H), 4.84- |
| | | | | 4.62 (m, 1H), 4.36-4.17 (m, 2H), 3.75 (d, J = |
| | | | | 11.7 Hz, 2H), 3.32 (s, 3H), 2.98-2.82 (m, 5H), |
| | | | | 2.74-2.58 (m, 5H), 2.11-1.95 (m, 4H), 1.94-1.82 |
| | | | | (m, 2H), 1.81-1.61 (m, 3H), 1.47-1.21 (m, 3H), |
| | | | | 1.16-1.02 (m, 2H), 0.90-0.79 (m, 1H), 0.64-0.51 |
| | | | | (m, 1H) |
| K660 | JS | IS | 922.5 | 11.06 (s, 1H), 8.81 (d, J = 4.2 Hz, 1H), 8.64- |
| | | | | 8.56 (m, 2H), 7.88-7.79 (m, 3H), 7.75 (dd, J = |
| | | | | 7.9, 1.7 Hz, 1H), 7.51-7.46 (m, 2H), 7.24 (t, J = |
| | | | | 7.9 Hz, 1H), 6.94 (d, J = 8.6 Hz, 1H), 6.84 (d, J = |
| | | | | 2.2 Hz, 1H), 6.62 (dd, J = 8.7, 2.2 Hz, 1H), |
| | | | | 6.13 (s, 1H), 5.29 (dd, J = 12.9, 5.3 Hz, 1H), |
| | | | | 4.84-4.61 (m, 1H), 3.68 (s, 2H), 3.51 (s, 3H), |
| | | | | 3.31 (s, 3H), 3.11-3.02 (m, 5H), 2.97-2.82 (m, |
| | | | | 12H), 2.74-2.57 (m, 2H), 2.44-2.29 (m, 1H), |
| | | | | 2.02-1.94 (m, 1H), 1.91-1.76 (m, 2H), 1.13-1.01 |
| | | | | (m, 1H), 0.72-0.59 (m, 1H) |
| K661 | JC | IN | 918.4 | 11.05 (s, 1H), 8.92-8.86 (m, 1H), 8.64-8.59 (m, |
| | | | | 1H), 7.96 (s, 1H), 7.87-7.80 (m, 1H), 7.80-7.74 |
| | | | | (m, 2H), 7.74-7.66 (m, 1H), 7.37-7.30 (m, 1H), |
| | | | | 7.34-7.26 (m, 1H), 6.97-6.91 (m, 1H), 6.86-6.81 |
| | | | | (m, 1H), 6.66-6.59 (m, 1H), 6.02 (s, 1H), 5.29 |
| | | | | (dd, J = 12.9, 5.4 Hz, 1H), 5.02-4.74 (m, 1H), |
| | | | | 4.20-4.11 (m, 2H), 3.68 (s, 2H), 3.50-3.41 (m, |
| | | | | 2H), 3.32 (s, 3H), 3.10-3.00 (m, 6H), 3.00-2.84 |
| | | | | (m, 10H), 2.76-2.53 (m, 2H), 2.44-2.29 (m, 1H), |
| | | | | 2.26-2.16 (m, 1H), 2.03-1.95 (m, 1H), 1.85-1.81 |
| | | | | (m, 2H), 1.30-1.16 (m, 1H), 1.05-0.90 (m, 1H) |
| K662 | KH | HT | 881.4 | 11.11 (s, 1H), 8.92 (d, J = 4.8 Hz, 1H), 7.94 (s, |
| | | | | 1H), 7.70-7.63 (m, 1H), 7.54 (d, J = 8.3 Hz, 2H), |
| | | | | 7.41 (d, J = 1.7 Hz, 1H), 7.34-7.24 (m, 2H), |
| | | | | 7.17-7.07 (m, 2H), 7.04-6.98 (m, 2H), 6.59 (d, J = |
| | | | | 8.1 Hz, 1H), 5.98 (s, 1H), 5.39 (dd, J = 12.8, |
| | | | | 5.4 Hz, 1H), 4.97-4.77 (m, 1H), 4.13-4.05 (m, |
| | | | | 2H), 3.79-3.71 (m, 2H), 3.40 (s, 3H), 3.09-3.02 |
| | | | | (m, 3H), 3.01-2.87 (m, 8H), 2.80-2.61 (m, 4H), |
| | | | | 2.58-2.52 (m, 4H), 2.29-2.22 (m, 2H), 2.08-1.99 |
| | | | | (m, 1H), 1.87-1.80 (m, 2H), 1.78-1.69 (m, 1H), |
| | | | | 1.31-1.18 (m, 3H), 1.01-0.90 (m, 1H) |

^{a}The reductive amination was run from 25-50° C. under standard conditions. KOAc was added to free base amines when necessary. DMSO/DCE was also employed as a solvent mixture. Purification was run under a variety of typical methods including prep-HPLC, reverse phase chromatography, and silica gel chromatography.
^{b}LCMS data reported as the (M − H)⁻ ion.
^{c}Ketone not aldehyde used for the reductive amination.

### Example 2.3. Method 3: Synthesis of N-{4-[1-(2,6-dioxopiperidin-3-yl)-3-methyl-2-oxo-1,3-benzodiazol-4-yl]butyl}-4-{3-[(3-{[(1R,2S)-2-fluorocyclopropyl]carbamoyl}-8-(methylamino)imidazo[1,2-b]pyridazin-6-yl)amino]-2-oxopyridin-1-yl}piperidine-1-carboxamide

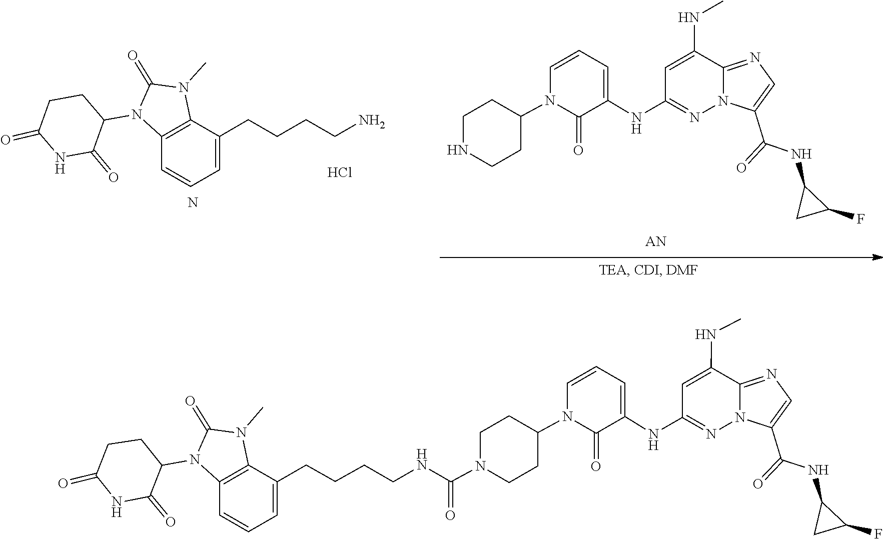

To a stirred mixture of 3-[4-(4-aminobutyl)-3-methyl-2-oxo-1,3-benzodiazol-1-yl]piperidine-2,6-dione hydrochloride (110.90 mg, 0.303 mmol, Intermediate N) and TEA (0.13 mL, 0.909 mmol) in DMF (2 mL) was added CDI (49.08 mg, 0.303 mmol) at rt under nitrogen atmosphere. The resulting mixture was stirred for 30 min at rt under nitrogen atmosphere. To the above mixture was then added N-[(1R,2S)-2-fluorocyclopropyl]-8-(methylamino)-6-{[2-oxo-1-(piperidin-4-yl)pyridin-3-yl]amino}imidazo[1,2-b]pyridazine-3-carboxamide (146 mg, 0.333 mmol, Intermediate AN) at rt. The resulting mixture was stirred for additional 16 h at rt. On completion, the reaction mixture was concentrated under reduced pressure. The residue was purified by reverse flash chromatography (column, silica gel; mobile phase A: water (0.1% FA), mobile phase B: MeCN, 15% to 40% gradient in 30 min; detector, UV 220/254 nm; desired fractions were collected at 23% B) and concentrated under reduced pressure to afford the title compound (56.9 mg, 23% yield) as a white solid. ¹H NMR (400 MHz, DMSO-d₆) δ 11.08 (s, 1H), 8.64 (d, J=4.2 Hz, 1H), 8.28 (s, 1H), 8.03-7.96 (m, 1H), 7.91-7.85 (m, 1H), 7.86 (s, 1H), 7.55-7.47 (m, 1H), 7.02-6.91 (m, 3H), 6.91-6.84 (m, 1H), 6.51 (t, J=5.5 Hz, 1H), 6.07 (s, 1H), 5.41-5.32 (m, 1H), 5.32-5.22 (m, 1H), 4.87-4.61 (m, 1H), 3.67-3.58 (m, 2H), 3.55 (s, 3H), 3.15-3.02 (m, 4H), 2.93-2.80 (m, 5H), 2.87 (s, 2H), 2.78-2.66 (m, 1H), 2.66-2.57 (m, 1H), 2.04-1.95 (m, 1H), 1.86 (s, 2H), 1.62-1.44 (m, 6H), 1.18-1.04 (m, 1H), 0.70-0.55 (m, 1H); LC/MS (ESI, m/z): [(M+H)]⁺=797.4.

TABLE E
Compounds synthesized via Method 3, via the coupling of the corresponding amines.

| | | | LCMS | |
|---|---|---|---|---|
| | | | (ESI+) | |
| Compound | | | m/z | |
| ID (K#) | Amine | Amine | (M + H)⁺ | ¹H NMR (400 MHz, DMSO-d6) |
| K277 | AN | AQ | 797.4 | 11.07 (s, 1H), 8.63 (d, J = 4.2 Hz, 1H), 8.28 (s, |
| | | | | 1H), 7.99 (dd, J = 7.6, 1.7 Hz, 1H), 7.91-7.80 (m, |
| | | | | 2H), 7.53-7.48 (m, 1H), 7.06-6.93 (m, 3H), 6.86 |
| | | | | (dd, J = 8.1, 1.5 Hz, 1H), 6.49 (t, J = 5.5 Hz, 1H), |
| | | | | 6.06 (s, 1H), 5.42-5.16 (m, 2H), 4.86-4.66 (m, 1H), |
| | | | | 3.68-3.61 (m, 2H), 3.43-3.31 (m, 3H), 3.19-2.97 |
| | | | | (m, 3H), 2.93-2.83 (m, 5H), 2.78-2.57 (m, 4H), |
| | | | | 2.04-1.94 (m, 1H), 1.91-1.82 (m, 2H), 1.60-1.52 |
| | | | | (m, 5H), 1.47-1.34 (m, 2H), 1.16-1.07 (m, 1H), |
| | | | | 0.68-0.57( m, 1H). |
| K330 | AN | CM | 797.5 | 11.08 (s, 1H), 8.64 (s, 1H), 8.20-8.17 (m, 2H), |
| | | | | 7.93-7.87 (m, 2H), 7.81 (d, J = 4.6 Hz, 1H), 6.98- |
| | | | | 6.93 (m, 3H), 6.90-6.85 (m, 1H), 6.52 (t, J = 5.5 |
| | | | | Hz, 1H), 5.90 (s, 1H), 5.36 (dd, J = 12.6, 5.4 Hz, |
| | | | | 1H), 5.31-5.24 (m, 1H), 4.88-4.65 (m, 1H), 3.74- |
| | | | | 3.66 (m, 2H), 3.56 (s, 3H), 3.14-3.05 (m, 4H), 2.93 |
| | | | | (d, J = 4.9 Hz, 3H), 2.91-2.84 (m, 3H), 2.77-2.67 |
| | | | | (m, 1H), 2.66-2.53 (m, 2H), 2.04-1.96 (m, 1H), |
| | | | | 1.93-1.85 (m, 2H), 1.64-1.48 (m, 6H), 1.18-1.08 |
| | | | | (m, 1H), 0.73-0.62 (m, 1H) |
| K396 | CC | I | 852.4 | 11.09 (s, 1H), 8.62 (d, J = 4.2 Hz, 1H), 8.26 (s, |
| | | | | 1H), 7.98 (d, J = 7.6 Hz, 1H), 7.90-7.87 (m, 1H), |
| | | | | 7.86 (s, 1H), 7.51-7.46 (m, 1H), 7.18-7.06 (m, 1H), |
| | | | | 7.00-6.94 (m, 2H), 6.92-6.86 (m, 1H), 6.07 (s, 1H), |
| | | | | 5.43-5.35 (m, 1H), 5.31-5.22 (m, 1H), 4.85-4.64 |
| | | | | (m, 1H), 3.68 (s, 3H), 3.64 (s, 2H), 3.53-3.41 (m, |
| | | | | 2H), 3.00-2.94 (m, 2H), 2.91 (d, J = 4.8 Hz, 3H), |
| | | | | 2.88-2.82 (m, 2H), 2.75-2.64 (m, 2H), 2.61 (s, 3H), |
| | | | | 2.11-1.87 (m, 6H), 1.71-1.58 (m, 4H), 1.56-1.52 |
| | | | | (m, 2H), 1.29-1.17 (m, 2H), 1.16-1.06 (m, 1H), |
| | | | | 0.69-0.54 (m, 1H) |
| K397 | CC | AN | 866.4 | 11.12 (s, 1H), 9.40 (s, 1H), 8.65-8.60 (m, 1H), |
| | | | | 8.33-8.28 (m, 1H), 8.00-7.94 (m, 1H), 7.91-7.85 |
| | | | | (m, 2H), 7.49-7.43 (m, 1H), 7.27 (d, J = 7.6 Hz, |
| | | | | 1H), 7.22-7.13 (m, 2H), 6.99-6.95 (m, 1H), 6.08- |
| | | | | 6.03 (m, 1H), 5.63-5.40 (m, 1H), 5.30-5.02 (m, |
| | | | | 1H), 4.83-4.63 (m, 1H), 4.61-4.55 (m, 2H), 3.58- |
| | | | | 3.50 (m, 3H), 3.51-3.44 (m, 3H), 3.31-3.27 (m, |
| | | | | 2H), 3.31-3.27 (m, 1H), 2.91-2.88 (m, 2H), 2.87- |
| | | | | 2.81 (m, 2H), 2.80-2.76 (m, 2H), 2.75- 2.70 (m, |
| | | | | 2H), 2.67-2.62 (m, 2H), 2.53-2.49 (m, 3H), 1.98- |
| | | | | 1.74 (m, 4H), 1.64-1.60 (m, 2H), 1.31-1.22 (m, |
| | | | | 2H), 1.14-1.06 (m, 2H), 1.06-1.02 (m, 1H), 0.60- |
| | | | | 0.53 (m, 1H) |
| K398 | CB | AN | 852.5 | 11.04 (s, 1H), 8.63 (d, J = 4.3 Hz, 1H), 8.28 (s, |
| | | | | 1H), 8.03-7.96 (m, 1H), 7.92-7.83 (m, 2H), 7.51- |
| | | | | 7.43 (m, 1H), 7.02-6.95 (m, 1H), 6.93 (d, J = 8.6 |
| | | | | Hz, 1H), 6.83 (d, J = 2.2 Hz, 1H), 6.67-6.60 (m, |
| | | | | 1H), 6.08 (s, 1H), 5.33-5.23 (m, 2H), 4.84-4.65 (m, |
| | | | | 1H), 3.39-3.35 (m, 2H), 3.32-3.30 (m, 5H), 3.11- |
| | | | | 3.05 (m, 2H), 3.03-2.93 (m, 2H), 2.92-2.83 (m, |
| | | | | 5H), 2.81-2.75 (m, 3H), 2.73-2.56 (m, 4H), 2.03- |
| | | | | 1.88 (m, 3H), 1.74-1.60 (m, 5H), 1.31-1.17 (m, |
| | | | | 2H), 1.17-1.07 (m, 1H), 0.68-0.58 (m, 1H) |
| K399 | E | AN | 881.5 | 11.05 (s, 1H), 8.63 (d, J = 4.3 Hz, 1H), 8.26 (s, |
| | | | | 1H), 7.99 (d, J = 7.6 Hz, 1H), 7.91-7.84 (m, 2H), |
| | | | | 7.51-7.43 (m, 1H), 7.03-6.93 (m, 3H), 6.84 (d, J = |
| | | | | 8.0 Hz, 1H), 6.43 (t, J = 5.5 Hz, 1H), 6.07 (s, 1H), |
| | | | | 5.33 (dd, J = 12.7, 5.4 Hz, 1H), 4.85-4.62 (m, 1H), |
| | | | | 3.68-3.56 (m, 2H), 3.32 (s, 3H), 3.15-3.03 (m, 2H), |
| | | | | 3.02-2.94 (m, 2H), 2.92-2.82 (m, 5H), 2.79-2.53 |
| | | | | (m, 5H), 2.06-1.96 (m, 1H), 1.92-1.81 (m, 2H), |
| | | | | 1.62-1.51 (m, 4H), 1.43-1.33 (m, 2H), 1.29-1.19 |
| | | | | (m, 12H), 1.16-1.04 (m, 1H), 0.70-0.55 (m, 1H) |

^{a}The reaction was run from 25-40° C. for 30 min to 2 hr. Purification was run under a variety of typical methods including prep-HPLC, reverse phase chromatography, and silica gel chromatography.
^{b}Triphosgene used in place of CDI, with TEA in a mixed solvent of DCM/DMF.

### Example 2.4. Method 4: Synthesis of 6-((5-(4-(1-(2,6-dioxopiperidin-3-yl)-3-methyl-2-oxo-2,3-dihydro-1H-benzo[d]imidazol-5-yl)piperazin-1-yl)pyridin-2-yl)amino)-4-((3-(5-fluoropyrimidin-2-yl)-2-methoxyphenyl)amino)-N-methylnicotinamide

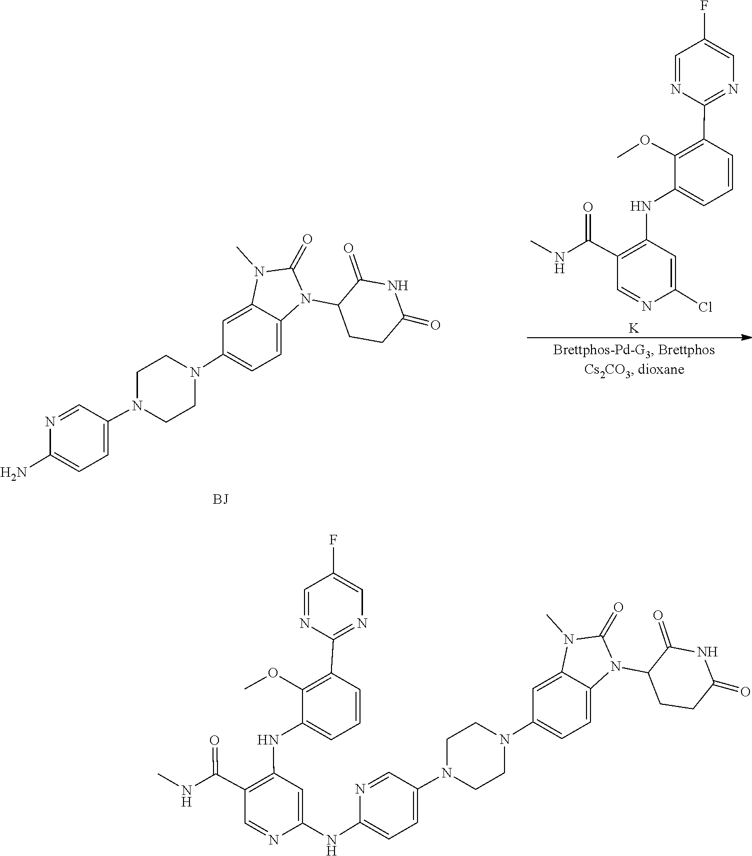

To a stirred solution of 3-(5-(4-(6-aminopyridin-3-yl)piperazin-1-yl)-3-methyl-2-oxo-2,3-dihydro-1H-benzo[d]imidazol-1-yl)piperidine-2,6-dione (50.00 mg, 0.115 mmol, Intermediate BJ) and 6-chloro-4-{[3-(5-fluoropyrimidin-2-yl)-2-methoxyphenyl]amino}-N-methylpyridine-3-carboxamide (44.52 mg, 0.115 mmol, Intermediate K) in 1,4-dioxane (2 mL) were added BrettPhos Pd G3 (20.82 mg, 0.023 mmol), BrettPhos (12.33 mg, 0.023 mmol) and Cs₂CO₃ (74.82 mg, 0.230 mmol) at rt under nitrogen atmosphere. The resulting mixture was stirred under nitrogen atmosphere at 120° C. for 16 h. On completion, the reaction mixture was cooled to rt and filtered. The filter cake was washed with MeCN (3×5 mL) and the filtrate was concentrated under reduced pressure. The crude product was purified by Prep-HPLC (Column: Xselect CSH C18 OBD Column 30*150 mm 5 μm, n; Mobile Phase A: ACN, Mobile Phase B: Water (0.1% FA); Flow rate: 60 mL/min; Gradient: 26% B to 38% B in 10 min, 38% B to 38% B in 12 min, 38% B; Wave Length: 254\220 nm; RT1 (min): 11.35) to afford the title compound (5.00 mg, 6% yield) as a light yellow solid. ¹H NNMR (400 MHz, DMSO-d₆) δ 11.07 (s, 1H), 10.69 (s, 1H), 9.51 (s, 1H), 9.19-8.92 (m, 2H), 8.51-8.41 (d, 2H), 7.94 (d, J=12.9 Hz, 1H), 7.81 (s, 1H), 7.75-7.68 (m, 1H), 7.54 (d, J=9.0 Hz, 1H), 7.51-7.45 (m, 1H), 7.44-7.38 (m, 1H), 7.37-7.29 (m, 1H), 6.98 (d, J=8.6 Hz, 1H), 6.95-6.89 (m, 1H), 6.75-6.65 (m, 1H), 5.45-5.16 (m, 1H), 3.70 (s, 3H), 3.39-3.31 (m, 8H), 3.25 (s, 3H), 3.01-2.82 (m, 1H), 2.79 (d, J=4.4 Hz, 3H), 2.74-2.57 (m, 2H), 2.04-1.95 (m, 1H). LC/MS (ESI, m/z): [(M+H)]⁺=787.4.

TABLE F
Compounds synthesized via Method 4, via the coupling of the
corresponding amines and activated chlorides or triflates.

| | | | LCMS | |
|---|---|---|---|---|
| | | | (ESI+) | |
| Compound | | Chloride/ | m/z | |
| ID (K#) | Amine | Triflate | (M + H)⁺ | ¹H NMR (400 MHz, DMSO-d6) |
| K204 | N | CK | 734.3 | 11.08 (s, 1H), 8.69 (d, J = 4.2 Hz, 1H), 8.65- |
| | | | | 8.54 (m, 2H), 8.07-7.87 (m, 3H), 7.80 (d, J = 8.1 |
| | | | | Hz, 1H), 7.56-7.41 (m, 3H), 6.99-6.82 (m, 3H), |
| | | | | 6.45-6.39 (m, 2H), 5.31 (dd, J = 12.8, 5.4 Hz, |
| | | | | 1H), 4.98-4.72 (m, 1H), 3.53 (s, 3H), 3.36-3.31 |
| | | | | (m, 2H), 3.04-2.77 (m, 4H), 2.76-2.57 (m, 2H), |
| | | | | 2.07-1.91 (m, 1H), 1.81-1.60 (m, 4H), 1.33-1.08 |
| | | | | (m, 1H), 1.03-0.85 (m, 1H) |
| K205 | N | CK | 734.2 | 11.07 (s, 1H), 8.67 (s, 1H), 8.65 (d, J = 4.3 Hz, |
| | | | | 2H), 8.08-8.02 (m, 1H), 7.99 (dd, J= 7.4, 1.7 |
| | | | | Hz, 1H), 7.92 (s, 1H), 7.87-7.83 (m, 1H), 7.56- |
| | | | | 7.51 (m, 3H), 7.05-6.98 (m, 2H), 6.89 (dd, J = |
| | | | | 8.0, 1.5 Hz, 1H), 6.55-6.50 (m, 1H), 6.41 (t, J = |
| | | | | 7.2 Hz, 1H), 5.32 (dd, J = 12.6, 5.4 Hz, 1H), |
| | | | | 5.01-4.78 (m, 1H), 3.30 (s, 3H), 3.05-2.96 (m, |
| | | | | 1H), 2.94-2.84 (m, 1H), 2.74-2.65 (m, 3H), |
| | | | | 2.64-2.52 (m, 3H), 2.03-1.95 (m, 1H), 1.69-1.66 |
| | | | | (m, 2H), 1.29-1.23 (m, 2H), 1.04-0.81 (m, 2H) |
| K201 | DP | CK | 794.4 | 11.06 (s, 1H), 8.69-8.59 (m, 3H), 8.07-8.02 (m, |
| | | | | 1H), 7.99 (dd, J = 7.4, 1.8 Hz, 1H), 7.93 (s, 1H), |
| | | | | 7.83 (d, J = 8.1 Hz, 1H), 7.58-7.50 (m, 2H), 7.28 |
| | | | | (t, J = 5.8 Hz, 1H), 7.06-7.01 (m, 1H), 6.97 (d, J = |
| | | | | 8.1 Hz, 1H), 6.87 (dd, J = 8.1, 1.6 Hz, 1H), |
| | | | | 6.53 (s, 1H), 6.41 (t, J = 7.2 Hz, 1H), 5.32 (dd, J = |
| | | | | 12.9, 5.4 Hz, 1H), 4.99-4.78 (m, 1H), 3.66 (t, |
| | | | | J = 5.9 Hz, 2H), 3.63-3.57 (m, 4H), 3.56-3.51 |
| | | | | (m, 2H), 3.46-3.41 (m, 2H), 3.29 (s, 3H), 3.04- |
| | | | | 2.96 (m, 1H), 2.95-2.77 (m, 3H), 2.72-2.57 (m, |
| | | | | 2H), 2.03-1.95 (m, 1H), 1.26-1.23 (m, 1H), |
| | | | | 1.05-0.92 (m, 1H) |
| K202 | AM | CK | 882.45 | 11.04 (s, 1H), 8.66-8.59 (m, 3H), 8.07-7.95 (m, |
| | | | | 2H), 7.93 (s, 1H), 7.83 (d, J = 8.1 Hz, 1H), 7.56- |
| | | | | 7.48 (m, 2H), 7.30-7.22 (m, 1H), 7.04 (s, 1H), |
| | | | | 6.98 (d, J = 8.0 Hz, 1H), 6.94-6.84 (m, 1H), 6.52 |
| | | | | (s, 1H), 6.41 (t, J = 7.2 Hz, 1H), 5.32 (dd, J = |
| | | | | 12.9, 5.4 Hz, 1H), 4.99-4.77 (m, 1H), 3.66 (t, J = |
| | | | | 5.8 Hz, 2H), 3.59-3.54 (m, 4H), 3.53 -3.51 (m, |
| | | | | 2H), 3.49-3.44 (m, 10H), 3.30 (s, 3H), 3.05-2.95 |
| | | | | (m, 1H), 2.94-2.84 (m, 1H), 2.80 (t, J = 7.0 Hz, |
| | | | | 2H), 2.76-2.57 (m, 2H), 2.05-1.94 (m, 1H), |
| | | | | 1.26-1.20 (m, 1H), 1.06-0.91 (m, 1H) |
| K203 | AP | CK | 970.5 | 11.08 (s, 1H), 8.72-8.56 (m, 3H), 8.08-7.95 (m, |
| | | | | 2H), 7.92 (s, 1H), 7.82 (d, J = 8.1 Hz, 1H), 7.56- |
| | | | | 7.47 (m, 2H), 7.27 (t, J = 6.0 Hz, 1H), 7.05 (s, |
| | | | | 1H), 7.01-6.83 (m, 2H), 6.51 (s, 1H), 6.41 (t, J = |
| | | | | 7.2 Hz, 1H), 5.32 (dd, J = 12.9, 5.4 Hz, 1H), |
| | | | | 4.98-4.76 (m, 1H), 4.10-3.96 (m, 2H), 3.68-3.62 |
| | | | | (m, 6H), 3.61-3.53 (m, 16H), 3.30 (s, 3H), 3.19- |
| | | | | 3.10 (m, 1H), 3.04-2.96 (m, 1H), 2.93-2.86 (m, |
| | | | | 1H), 2.81 (t, J = 7.0 Hz, 2H), 2.75-2.57 (m, 3H), |
| | | | | 2.05-1.94 (m, 1H), 1.27-1.21 (m, 1H), 1.03-0.91 |
| | | | | (m, 1H) |
| K702 | DS | K | 787.4 | 11.11 (s, 1H), 10.70 (s, 1H), 9.58 (s, 1H), 9.07- |
| | | | | 9.03 (m, 2H), 8.52-8.47 (m, 2H), 7.96-7.92 (m, |
| | | | | 1H), 7.82-7.75 (m, 2H), 7.56-7.45 (m, 2H), |
| | | | | 7.44-7.39 (m, 1H), 7.38-7.32 (m, 1H), 7.07-6.97 |
| | | | | (m, 2H), 6.96-6.90 (m, 1H), 5.38 (dd, J = 12.7, |
| | | | | 5.4 Hz, 1H), 3.71 (s, 3H), 3.67 (s, 3H), 3.33- |
| | | | | 3.26 (m, 4H), 3.22-2.85 (m, 5H), 2.79 (d, J = 4.4 |
| | | | | Hz, 3H), 2.75-2.58 (m, 2H), 2.07-1.97 (m, 1H) |

^{a}The coupling was run under standard conditions, from 90-120° C. for 16 hrs. Other bases such as tBuONa and K₂CO₃ could also be employed. Purification was run under a variety of typical methods including prep-HPLC, reverse phase chromatography, and silica gel chromatography.

### Example 2.5. Method 5: Synthesis of 6-[({6-[1-(2,6-dioxopiperidin-3-yl)-3-methyl-2-oxo-1,3-benzodiazol-4-yl]hexyl}carbamoyl)amino]-4-{[3-(5-fluoropyrimidin-2-yl)-2-methoxyphenyl]amino}-N-methylpyridine-3-carboxamide

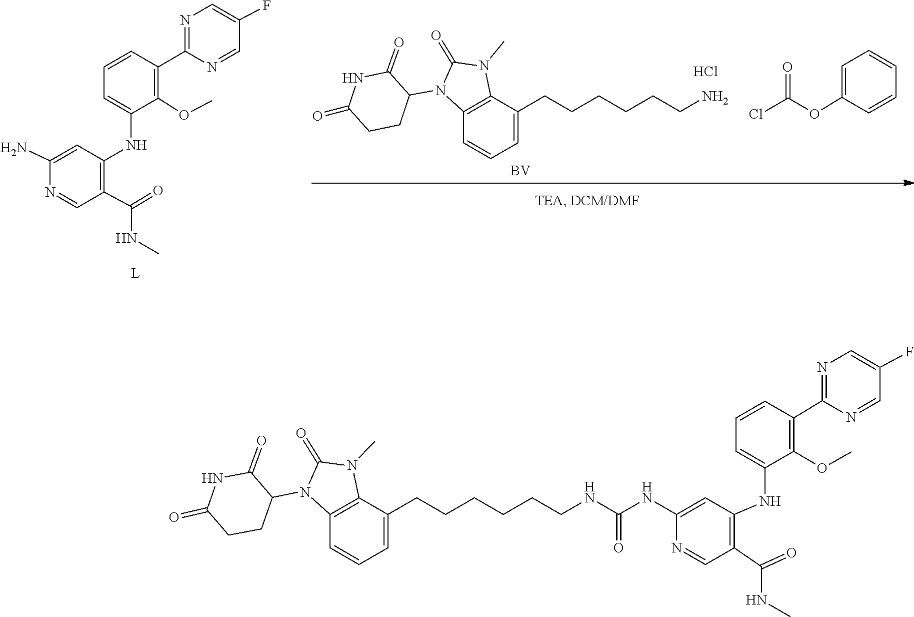

To a stirred solution of 6-amino-4-{[3-(5-fluoropyrimidin-2-yl)-2-methoxyphenyl]amino}-N-methylpyridine-3-carboxamide (50 mg, 0.1 mmol, Intermediate L) in DCM (2 mL) and DMA (2 mL) was added phenyl chloroformate (25.50 mg, 0.163 mmol) at rt. The resulting mixture was stirred for 30 min at rt. To the above mixture was added TEA (0.06 mL, 0.408 mmol) and 3-[4-(6-aminohexyl)-3-methyl-2-oxo-1,3-benzodiazol-1-yl]piperidine-2,6-dione hydrochloride (453.58 mg, 0.136 mmol, Intermediate BV) at rt. The resulting mixture was stirred for an additional 1 h at rt. On completion, the reaction mixture was concentrated under reduced pressure. The residue was purified by Prep-HPLC (Column: Xselect CSH C18 OBD Column 30*150 mm 5 μm, n; Mobile Phase A: Water (10 mmol/L NH₄HCO₃), Mobile Phase B: ACN; Flow rate: 60 mL/min; Gradient: 35% B to 47% B in 12 min, 47% B; Wave Length: 220 nm; RT1 (min): 8.78) to afford the title compound (10.6 mg, 10% yield) as a white solid. ¹H NMR (400 MHz, DMSO-d₆) δ 11.08 (s, 1H), 10.73 (s, 1H), 9.05 (s, 2H), 9.12 (s, 1H), 8.78-8.63 (m, 1H), 8.40 (s, 1H), 8.03 (s, 1H), 7.58 (dd, J=8.0, 1.6 Hz, 1H), 7.55-7.49 (m, 1H), 7.31 (t, J=7.8 Hz, 1H), 7.18-7.11 (m, 1H), 7.02-6.92 (m, 2H), 6.89-6.83 (m, 1H), 5.36 (dd, J=12.6, 5.4 Hz, 1H), 3.68 (s, 3H), 3.55 (s, 3H), 3.20-3.08 (m, 2H), 2.96-2.84 (m, 3H), 2.80 (d, J=4.4 Hz, 3H), 2.75-2.58 (m, 2H), 2.04-1.95 (m, 1H), 1.68-1.55 (m, 2H), 1.53-1.30 (m, 6H). LC/MS (ESI, m/z): [(M+H)]⁺=753.3.

TABLE G
Compounds synthesized via Method 5, via the coupling of the corresponding amines.

| | | | LCMS | |
|---|---|---|---|---|
| | | | (ESI+) | |
| Compound | | | m/z | |
| ID (K#) | Amine | Acid | (M + H)⁺ | ¹H NMR (400 MHz, DMSO-d6) |
| K188 | DJ | L | 767.3 | 11.07 (s, 1H), 10.61 (s, 1H), 9.08 (s, 1H), 9.05-9.02 |
| | | | | (m, 2H), 8.57-8.50 (m, 1H), 8.42 (s, 1H), 8.04-7.97 |
| | | | | (m, 1H), 7.57 (dd, J = 8.0, 1.7 Hz, 1H), 7.43 (dd, J = |
| | | | | 7.8, 1.6 Hz, 1H), 7.33 (s, 1H), 7.27 (t, J = 7.9 Hz, |
| | | | | 1H), 7.04-6.94 (m, 2H), 6.89-6.82 (m, 1H), 5.33 (dd, |
| | | | | J = 12.7, 5.3 Hz, 1H), 3.68 (s, 3H), 3.32 (s, 3H), |
| | | | | 3.17-3.07 (m, 2H), 2.97-2.84 (m, 1H), 2.78 (d, J = |
| | | | | 4.4 Hz, 3H), 2.72-2.58 (m, 4H), 2.04-1.95 (m, 1H), |
| | | | | 1.64-1.54 (m, 2H), 1.48-1.39 (m, 2H), 1.36-1.26 (m, |
| | | | | 6H) |
| K187 | CL | L | 781.4 | 11.06 (s, 1H), 10.61 (s, 1H), 9.08 (s, 1H), 9.05-9.01 |
| | | | | (m, 2H), 8.57-8.49 (m, 1H), 8.42 (s, 1H), 8.10-7.98 |
| | | | | (m, 1H), 7.59-7.53 (m, 1H), 7.46-7.40 (m, 1H), 7.34- |
| | | | | 7.24 (m, 2H), 7.04-6.96 (m, 2H), 6.90-6.81 (m, 1H), |
| | | | | 5.33 (dd, J = 12.7, 5.3 Hz, 1H), 3.68 (s, 3H), 3.32 (s, |
| | | | | 3H), 3.17-3.08 (m, 2H), 2.97-2.82 (m, 1H), 2.78 (d, J = |
| | | | | 4.3 Hz, 3H), 2.74-2.55 (m, 4H), 2.05-1.93 (m, 1H), |
| | | | | 1.64-1.53 (m, 2H), 1.49-1.38 (m, 2H), 1.35-1.22 (m, |
| | | | | 8H) |
| K186 | GU | L | 795.4 | 11.06 (s, 1H), 10.61 (s, 1H), 9.07 (s, 1H), 9.05-9.02 |
| | | | | (m, 2H), 8.55-8.50 (m, 1H), 8.42 (s, 1H), 8.07-7.98 |
| | | | | (m, 1H), 7.59-7.54 (m, 1H), 7.45-7.40 (m, 1H), 7.33- |
| | | | | 7.23 (m, 2H), 7.04-6.96 (m, 2H), 6.88-6.82 (d, J = |
| | | | | 8.0 Hz, 1H), 5.33 (dd, J = 12.7, 5.7 Hz, 1H), 3.68 (s, |
| | | | | 3H), 3.34 (s, 3H), 3.15-3.06 (m, 2H), 2.95-2.83 (m, |
| | | | | 1H), 2.78 (d, J = 4.5 Hz, 3H), 2.71-2.54 (m, 4H), |
| | | | | 2.05-1.93 (m, 1H), 1.62-1.51 (m, 2H), 1.48-1.38 (m, |
| | | | | 2H), 1.29 (m, 10H) |
| K185 | DL | L | 767.35 | 11.08 (s, 1H), 10.62 (s, 1H), 9.09 (s, 1H), 9.06-9.02 |
| | | | | (m, 2H), 8.54 (s, 1H), 8.43 (s, 1H), 8.03 (s, 1H), 7.57 |
| | | | | (dd, J = 8.0, 1.7 Hz, 1H), 7.43 (dd, J = 7.8, 1.6 Hz, |
| | | | | 1H), 7.33 (s, 1H), 7.27 (t, J = 7.9 Hz, 1H), 6.97-6.93 |
| | | | | (m, 2H), 6.86 (dd, J = 5.9, 3.1 Hz, 1H), 5.36 (dd, J = |
| | | | | 12.5, 5.5 Hz, 1H), 3.68 (s, 3H), 3.55 (s, 3H), 3.18- |
| | | | | 3.08 (m, 2H), 2.94-2.83 (m, 3H), 2.78 (d, J = 4.4 Hz, |
| | | | | 3H), 2.74-2.57 (m, 2H), 2.04-1.94 (m, 1H), 1.66-1.54 |
| | | | | (m, 2H), 1.50-1.41 (m, 2H), 1.41-1.28 (m, 6H) |
| K184 | CI | L | 781.4 | 11.07 (s, 1H), 10.61 (s, 1H), 9.08 (s, 1H), 9.06-9.00 |
| | | | | (m, 2H), 8.58-8.48 (m, 1H), 8.43 (s, 1H), 8.09-7.97 |
| | | | | (m, 1H), 7.60-7.52 (m, 1H), 7.46-7.39 (m, 1H), 7.32 |
| | | | | (s, 1H), 7.30-7.23 (m, 1H), 6.99-6.91 (m, 2H), 6.89- |
| | | | | 6.81 (m, 1H), 5.36 (dd, J = 12.5, 5.4 Hz, 1H), 3.68 (s, |
| | | | | 3H), 3.55 (s, 3H), 3.18-3.07 (m, 2H), 2.96-2.83 (m, |
| | | | | 3H), 2.78 (d, J = 4.4 Hz, 3H), 2.73-2.57 (m, 2H), |
| | | | | 2.04-1.96 (m, 1H), 1.65-1.54 (m, 2H), 1.50-1.40 (m, |
| | | | | 2H), 1.41-1.21 (m, 8H) |
| K183 | EH | L | 795.4 | 11.07 (s, 1H), 10.61 (s, 1H), 9.07 (s, 1H), 9.05-9.02 |
| | | | | (m, 2H), 8.56-8.48 (m, 1H), 8.42 (s, 1H), 8.08-7.95 |
| | | | | (m, 1H), 7.60-7.52 (m, 1H), 7.46-7.40 (m, 1H), 7.35- |
| | | | | 7.22 (m, 2H), 6.99-6.90 (m, 2H), 6.89-6.81 (m, 1H), |
| | | | | 5.42-5.28 (m, 1H), 3.68 (s, 3H), 3.55 (s, 3H), 3.17- |
| | | | | 3.05 (m, 2H), 2.95-2.82 (m, 3H), 2.78 (d, J = 4.4 Hz, |
| | | | | 3H), 2.74-2.57 (m, 2H), 2.05-1.94 (m, 1H), 1.66-1.52 |
| | | | | (m, 2H), 1.50-1.39 (m, 2H), 1.38-1.27 (m, 10H) |

^{a}The coupling was run under standard conditions at rt for 1-2 hrs. DCM alone could also be used as the only solvent. Purification was run under a variety of typical methods including prep-HPLC, reverse phase chromatography, and silica gel chromatography.

### Example 2.6. Method 6: Synthesis of N-[(1R,2S)-2-fluorocyclopropyl]-8-(methylamino)-6-[4-(4-{4-[(1r,4r)-4-[1-(2,6-dioxopiperidin-3-yl)-3-methyl-2-oxo-1,3-benzodiazol-5-yl]cyclohexyl]piperazin-1-yl}pyridin-2-yl)-2,3-dihydroindol-1-yl]imidazo[1,2-b]pyridazine-3-carboxamide (K650)

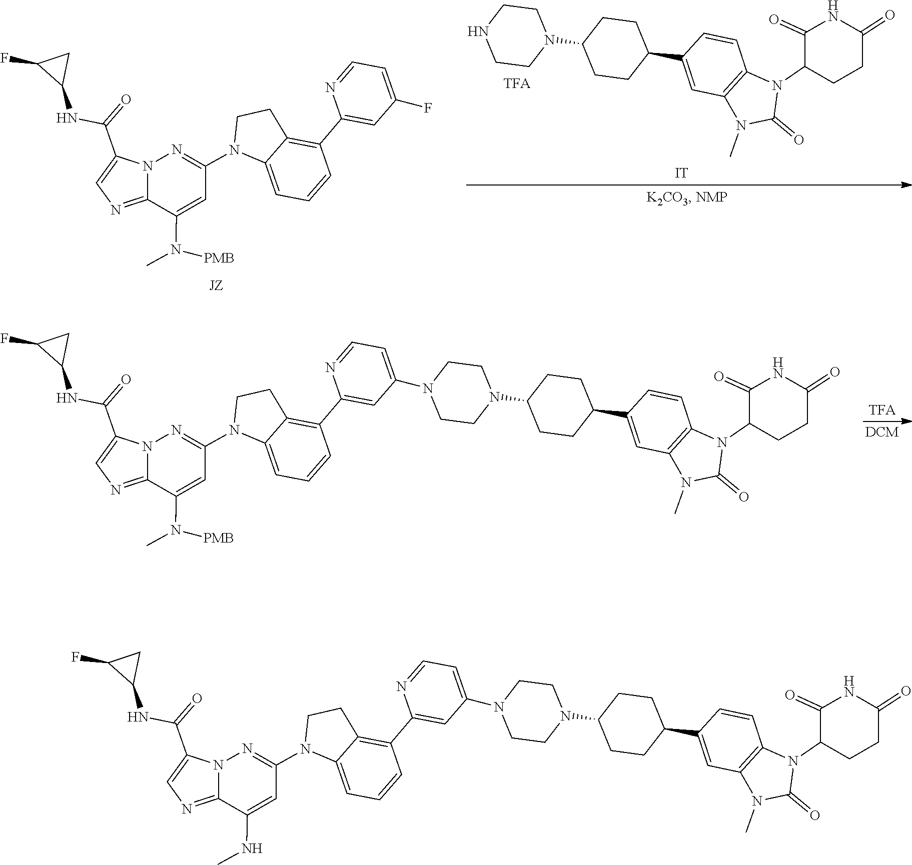

### Step 1—N-[(1R,2S-2-fluorocyclopropyl]-8-{[(4-methoxyphenyl)methyl](methyl)amino}-6-[4-(4-{4-[(1r,4r)-4-[1-(2,6-dioxopiperidin-3-yl)-3-methyl-2-oxo-1,3-benzodiazol-5-yl]cyclohexyl]piperazin-1-yl}pyridin-2-yl)-2,3-dihydroindol-1-yl]imidazo[1,2-b]pyridazine-3-carboxamide

To a stirred solution of N-[(1R,2S)-2-fluorocyclopropyl]-6-[4-(4-fluoropyridin-2-yl)-2,3-dihydroindol-1-yl]-8-{[(4-methoxyphenyl)methyl](methyl)amino}imidazo[1,2-b]pyridazine-3-carboxamide (30 mg, 0.052 mmol, Intermediate JZ) and 3-{3-methyl-2-oxo-5-[(1r,4r)-4-(piperazin-1-yl)cyclohexyl]-1,3-benzodiazol-1-yl}piperidine-2,6-dione (21.95 mg, 0.052 mmol, Intermediate IT) in DMSO (2 mL) was added DIEA (0.045 mL, 0.260 mmol) at rt. The resulting mixture was stirred for 16 h at 100° C. On completion, the mixture was cooled to rt and concentrated under reduced pressure. The residue was purified by reversed-phase flash chromatography (column, C18 silica gel; mobile phase, MeCN in water (10 mmol/L NH₄HCO₃), 20% to 50% gradient in 30 min; detector, UV 254 nm) to afford the title compound (40 mg, 79% yield) as a light brown oil. LC/MS (ESI, m/z): [(M+H)]⁺=987.5.

### Step 2—N-[(1R,2S)-2-fluorocyclopropyl]-8-(methylamino)-6-[4-(4-{4-[(1r,4r)-4-[1-(2,6-dioxopiperidin-3-yl)-3-methyl-2-oxo-1,3-benzodiazol-5-yl]cyclohexyl]piperazin-1-yl}pyridin-2-yl)-2,3-dihydroindol-1-yl]imidazo[1,2-b]pyridazine-3-carboxamide

A solution of N-[(1R,2S)-2-fluorocyclopropyl]-8-{[(4-methoxyphenyl)methyl](methyl)amino}-6-[4-(4-{4-[(1r,4r)-4-[1-(2,6-dioxopiperidin-3-yl)-3-methyl-2-oxo-1,3-benzodiazol-5-yl]cyclohexyl]piperazin-1-yl}pyridin-2-yl)-2,3-dihydroindol-1-yl]imidazo[1,2-b]pyridazine-3-carboxamide (30 mg, 0.030 mmol) and TFA (0.2 mL, 2.693 mmol) in DCM (1 mL) was stirred for 1 h at rt. On completion, the mixture was concentrated under reduced pressure. The crude product was purified by Prep-HPLC (Column: XBridge Prep OBD C18 Column, 30*150 mm, 5 μm; Mobile Phase A: water (10 mmol/L NH₄HCO₃), Mobile Phase B: ACN; Flow rate: 60 mL/min; Gradient: 35% B to 45% B in 10 min, 45% B; Wave Length: 220/254 nm; RT: 8.58 min) to afford the title compound (3.4 mg, 13% yield) as an off-white solid. ¹H NMR (400 MHz, DMSO-d₆) δ 11.10 (s, 1H), 8.90 (d, J=4.6 Hz, 1H), 8.28 (d, J=5.8 Hz, 1H), 7.96 (s, 1H), 7.75-7.67 (m, 2H), 7.29-7.23 (m, 2H), 7.12-7.04 (m, 2H), 7.00 (d, J=8.1 Hz, 1H), 6.91 (d, J=8.4 Hz, 1H), 6.83 (s, 1H), 6.01 (s, 1H), 5.35-5.31 (m, 1H), 5.02-4.86 (m, 1H), 4.15-4.08 (m, 2H), 3.42-3.36 (m, 6H), 3.07-3.03 (m, 1H), 3.00-2.95 (m, 3H), 2.92-2.88 (m, 1H), 2.72-2.66 (m, 6H), 2.49-2.46 (m, 1H), 2.00-1.85 (m, 6H), 1.59-1.49 (m, 2H), 1.47-1.44 (m, 3H), 1.27-1.21 (m, 2H), 1.05-0.99 (m, 1H), 0.94-0.89 (m, 1H); LC/MS (ESI, m/z): [(M+H)]⁺=867.5.

### Example 2.7. Method 7. Synthesis of 6-({3-[3-(4-{4-[1-(2,6-Dioxopiperidin-3-yl)-3-methyl-2-oxo-1,3-benzodiazol-5-yl]phenyl}piperazin-1-yl)prop-1-yn-1-yl]-2-methoxyphenyl}amino)-N-[(1R,2S)-2-fluorocyclopropyl]-8-(methylamino)imidazo[1,2-b]pyridazine-3-carboxamide (Example K543)

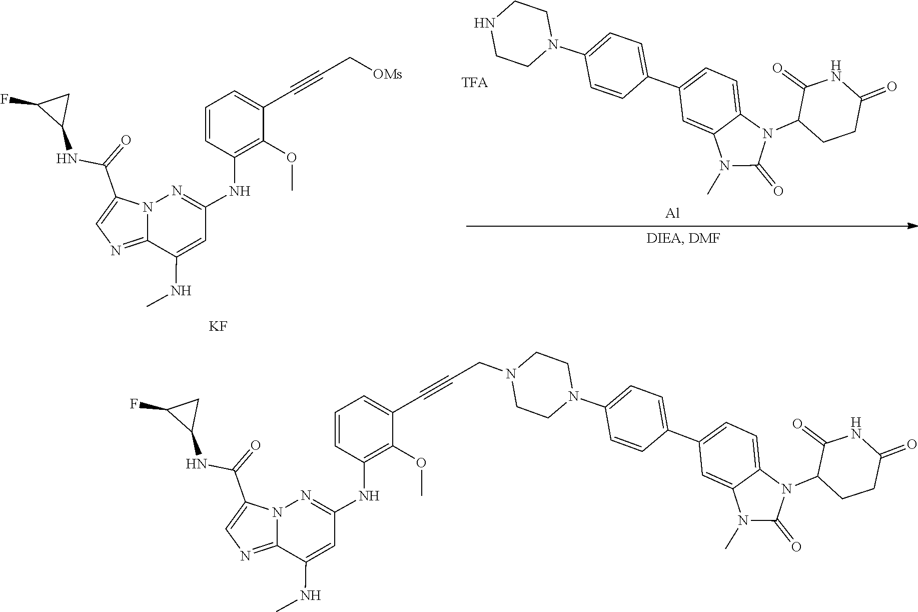

To a stirred solution of 3-{3-[(3-{[(1R,2S)-2-fluorocyclopropyl]carbamoyl}-8-(methylamino)imidazo[1,2-b]pyridazin-6-yl)amino]-2-methoxyphenyl}prop-2-yn-1-yl methanesulfonate (300 mg, 0.6 mmol, Intermediate KF) and 3-{3-methyl-2-oxo-5-[4-(piperazin-1-yl)phenyl]-1,3-benzodiazol-1-yl}piperidine-2,6-dione trifluoroacetate (370.74 mg, 0.716 mmol, Intermediate AI) in DMF (5 mL) was added DIEA (231.48 mg, 1.791 mmol) dropwise at rt under nitrogen atmosphere. The resulting mixture was stirred for 2 h at 100° C. under nitrogen atmosphere. On completion, the reaction mixture was cooled to rt and the mixture was concentrated under reduced pressure. The residue was purified by reverse phase flash chromatography (Column: WelFlash™ C18-I, 20-40 μm, 120 g; Eluent A: Water (plus 5 mmol/L NH₄HCO₃); Eluent B: ACN; Gradient: 30%-60% B in 35 min; Flow rate: 60 mL/min; Detector: 220/254 nm; desired fractions were collected at 51% B) and concentrated under reduced pressure to afford the title compound (73.9 mg, 14% yield) as a light brown solid. ¹H NMR (400 MHz, DMSO-d₆) δ 11.10 (s, 1H), 8.70 (d, J=4.2 Hz, 1H), 8.51 (s, 1H), 7.88-7.85 (m, 1H), 7.74-7.70 (m, 1H), 7.56 (d, J=8.5 Hz, 2H), 7.52-7.47 (m, 1H), 7.45-7.41 (m, 1H), 7.30-7.25 (m, 1H), 7.18-7.11 (m, 2H), 7.10-7.01 (m, 3H), 6.10 (s, 1H), 5.38 (dd, J=12.8, 5.4 Hz, 1H), 4.86-4.63 (m, 1H), 3.85 (s, 3H), 3.68 (s, 2H), 3.40 (s, 3H), 3.29-3.22 (m, 4H), 2.98-2.90 (m, 1H), 2.88 (d, J=4.9 Hz, 3H), 2.86-2.82 (m, 1H), 2.80-2.70 (m, 5H), 2.70-2.60 (m, 1H), 2.08-1.98 (m, 1H), 1.17-1.06 (m, 1H), 0.73-0.59 (m, 1H). LC/MS (ESI, m/z): [(M+H)]⁺=826.5.

### Example 3. TYK2 Degradation MSD Assay in EBV-B, OCI-LY10, and Human PBMC Cells

Degradation of TYK2 in cells was quantitatively measured using Meso Scale Discovery (MSD) technology. EBV-B, OCI-LY10 or human PBMC cells were seeded in 96-well plates with a density of 0.4 million cells per well in 100 μl fresh media. Compounds were then added to the assay plates with a final top concentration of 1 to 10 μM in a 1:3 dilution series with total of 9 doses. The assay plates were then incubated for 4 to 24 hours at 37° C. under 5% CO2. The assay plates were then centrifuged for 5 minutes, and the cell pellets were treated with 100 μl/well RIPA lysis buffer (Boston Bioproducts, BP-115D) with protease and phosphatase inhibitors (Roche, 05892791001 and 04906837001). To prepare MSD assay plates (MSD, L15XA), the plates were coated with the capture antibody (Santa Cruz Biotechnology, SC-5271) in PBS, at 40 μl/well. The plates were then incubated overnight at 4° C., washed 3 times with 150 μl/well TBST buffer (Cell Signaling Technology, 9997S) and blocked with 150 μl/well blocking buffer (MSD, R93BA-4). Cell lysates were then added to MSD assay plates and the plates were incubated at room temperature for 1 hour. The plates were then washed 3 times with 150 μl/well TBST buffer and 25 μl/well primary detection antibody (Cell Signaling Technology, #9312). The assay plates were then incubated at room temperature for 1 hour, washed 3 times with 150 μl/well TBST buffer, and 25 μl/well secondary detection antibody, SULFO-TAG anti-rabbit antibody (MSD, R32AB-1) were added. The assay plates were then incubated at room temperature for 1 hour, washed 3 times with 150 μl/well TBST buffer, and 150 μl/well MSD reading buffer (MSD, R92TC-2) were added. The plates were then analyzed by an MSD reader. The data was analyzed by GraphPad Prism and the dose dependent TYK2 degradation was fit using the four-parameter inhibitor vs. response nonlinear regression. The DC50 data were measured and categorized into different levels in Table H. The compound with DC50 less than or equal to 1 nM is marked as A, with DC50 more than 1 nM and less than or equal to 10 nM is marked as B, with DC50 more than 10 nM and less than or equal to 100 nM is marked as C, and with DC50 more than 100 nM or not available is marked as D.

TABLE H
TYK2 MSD DC50 ratings for the compounds
in EBV-B, OCI-LY10 and human PBMC cells

| | TYK2 | TYK2 | TYK2 |
|---|---|---|---|
| | MSD | MSD | MSD |
| | DC50 | DC50 | DC50 |
| | rating | rating | rating |
| | in | in | in |
| Compound | EBV- | OCI- | human |
| ID (K#) | B | LY10 | PBMC |
| K104 | D | D | D |
| K105 | D | D | D |
| K106 | D | D | D |
| K107 | D | D | A |
| K110 | D | D | A |
| K111 | B | D | A |
| K112 | B | D | B |
| K113 | D | D | B |
| K114 | B | A | A |
| K115 | A | D | A |
| K116 | B | D | B |
| K122 | B | D | B |
| K123 | C | D | B |
| K124 | C | D | B |
| K125 | B | D | B |
| K126 | D | D | A |
| K127 | D | D | A |
| K128 | D | D | B |
| K129 | B | D | A |
| K130 | B | D | B |
| K131 | A | A | A |
| K132 | A | D | A |
| K133 | B | D | D |
| K134 | A | D | A |
| K135 | A | D | A |
| K136 | A | D | B |
| K137 | A | D | A |
| K138 | A | D | A |
| K139 | A | D | A |
| K140 | A | D | B |
| K141 | D | A | A |
| K142 | A | D | B |
| K143 | D | D | A |
| K144 | A | D | A |
| K145 | B | D | B |
| K146 | A | D | A |
| K147 | A | D | B |
| K148 | A | D | A |
| K149 | A | D | D |
| K150 | A | D | A |
| K151 | A | D | D |
| K152 | B | D | D |
| K153 | D | A | A |
| K154 | D | A | A |
| K155 | A | D | A |
| K156 | D | D | D |
| K157 | A | D | D |
| K158 | A | D | D |
| K159 | A | D | D |
| K160 | A | D | D |
| K161 | B | D | D |
| K162 | B | D | D |
| K163 | D | D | D |
| K164 | A | D | D |
| K165 | B | D | D |
| K166 | A | D | D |
| K167 | A | D | D |
| K168 | D | D | D |
| K169 | B | D | D |
| K170 | A | D | D |
| K171 | A | D | D |
| K172 | A | D | D |
| K173 | A | D | D |
| K174 | B | D | D |
| K175 | A | D | A |
| K176 | B | D | A |
| K177 | A | D | A |
| K178 | A | D | A |
| K179 | D | D | C |
| K180 | C | D | B |
| K181 | D | D | B |
| K182 | C | D | B |
| K183 | D | D | B |
| K184 | D | D | B |
| K185 | D | D | B |
| K186 | D | D | B |
| K187 | D | D | B |
| K188 | D | D | A |
| K189 | D | C | C |
| K190 | C | D | B |
| K191 | D | B | B |
| K192 | D | B | B |
| K193 | B | D | B |
| K194 | D | D | A |
| K195 | B | D | D |
| K196 | D | A | A |
| K197 | D | D | A |
| K198 | D | D | A |
| K199 | D | D | B |
| K201 | D | D | D |
| K202 | D | D | D |
| K203 | D | D | D |
| K204 | D | D | D |
| K205 | D | D | D |
| K206 | D | D | D |
| K207 | D | D | D |
| K208 | D | D | D |
| K209 | D | D | D |
| K210 | D | D | D |
| K211 | D | D | D |
| K212 | D | D | D |
| K213 | D | D | D |
| K214 | D | D | D |
| K215 | D | D | C |
| K216 | D | D | B |
| K217 | C | D | C |
| K218 | D | D | C |
| K219 | D | D | B |
| K220 | D | D | D |
| K221 | D | D | D |
| K222 | D | D | B |
| K223 | D | D | B |
| K224 | D | D | C |
| K225 | D | D | B |
| K226 | D | D | C |
| K227 | D | D | D |
| K228 | D | D | B |
| K229 | D | D | A |
| K230 | D | D | B |
| K231 | D | D | A |
| K232 | D | D | B |
| K233 | B | D | B |
| K234 | D | D | D |
| K235 | D | D | B |
| K236 | D | D | B |
| K237 | D | D | D |
| K238 | D | D | D |
| K239 | D | D | D |
| K240 | D | D | D |
| K241 | D | D | D |
| K242 | D | D | D |
| K243 | D | D | D |
| K244 | D | D | D |
| K245 | D | D | D |
| K246 | D | D | D |
| K247 | D | D | D |
| K248 | D | D | D |
| K249 | D | D | D |
| K250 | D | D | A |
| K251 | D | D | A |
| K252 | D | D | D |
| K253 | D | D | D |
| K254 | D | D | B |
| K255 | D | D | A |
| K256 | D | D | A |
| K257 | D | C | B |
| K258 | D | D | A |
| K259 | D | D | D |
| K260 | D | D | A |
| K261 | B | D | A |
| K262 | D | D | B |
| K263 | D | D | B |
| K264 | D | D | D |
| K265 | D | D | A |
| K266 | D | D | A |
| K275 | D | D | D |
| K276 | D | D | C |
| K277 | D | D | B |
| K278 | D | D | B |
| K279 | D | D | A |
| K280 | A | D | A |
| K281 | A | D | A |
| K294 | D | D | D |
| K295 | D | D | D |
| K296 | D | D | A |
| K298 | D | D | D |
| K299 | D | D | D |
| K300 | D | D | D |
| K301 | D | D | D |
| K302 | D | D | D |
| K303 | D | D | D |
| K304 | D | D | D |
| K305 | D | D | D |
| K306 | D | D | D |
| K307 | D | D | D |
| K308 | D | D | D |
| K309 | D | D | D |
| K310 | D | D | D |
| K311 | D | D | D |
| K312 | D | D | D |
| K313 | D | D | B |
| K314 | D | D | D |
| K315 | D | D | D |
| K316 | D | D | D |
| K317 | D | D | D |
| K318 | D | D | D |
| K319 | D | D | D |
| K320 | D | D | D |
| K321 | D | D | D |
| K322 | D | D | D |
| K324 | D | D | D |
| K325 | D | D | D |
| K326 | D | D | D |
| K327 | D | D | D |
| K328 | D | D | D |
| K330 | D | D | D |
| K331 | D | D | A |
| K360 | D | D | D |
| K361 | D | D | D |
| K362 | D | D | A |
| K363 | D | D | A |
| K364 | D | D | D |
| K365 | D | D | D |
| K366 | D | D | D |
| K367 | D | D | D |
| K368 | D | D | D |
| K369 | D | D | B |
| K370 | A | A | D |
| K371 | B | D | D |
| K372 | A | D | D |
| K373 | A | D | D |
| K374 | A | D | D |
| K375 | B | D | D |
| K376 | C | D | D |
| K377 | A | D | D |
| K378 | A | D | D |
| K379 | A | D | D |
| K380 | A | A | A |
| K381 | A | A | D |
| K382 | A | D | D |
| K383 | A | D | A |
| K384 | D | D | D |
| K385 | A | D | A |
| K386 | D | D | A |
| K387 | D | D | A |
| K388 | D | D | D |
| K389 | A | A | A |
| K390 | D | D | A |
| K391 | A | D | A |
| K392 | D | D | A |
| K393 | D | D | A |
| K394 | B | D | D |
| K395 | A | D | D |
| K396 | B | D | D |
| K397 | B | D | D |
| K398 | A | D | D |
| K399 | B | D | D |
| K400 | D | D | D |
| K401 | D | D | D |
| K402 | D | D | D |
| K403 | D | D | D |
| K404 | D | D | D |
| K405 | D | D | D |
| K406 | D | D | D |
| K407 | D | A | D |
| K408 | D | A | D |
| K409 | D | B | D |
| K410 | D | A | D |
| K411 | D | A | D |
| K412 | D | D | D |
| K413 | D | A | D |
| K414 | D | A | D |
| K415 | B | D | D |
| K416 | D | D | D |
| K417 | D | D | D |
| K418 | D | A | D |
| K419 | D | A | D |
| K420 | D | A | D |
| K421 | D | B | D |
| K422 | D | A | D |
| K423 | D | B | D |
| K424 | D | D | D |
| K425 | D | D | D |
| K426 | D | D | D |
| K427 | D | A | D |
| K428 | D | A | D |
| K429 | B | A | D |
| K430 | B | A | D |
| K431 | D | B | D |
| K432 | D | A | D |
| K433 | D | D | D |
| K434 | D | D | D |
| K435 | D | A | D |
| K436 | D | A | D |
| K437 | D | A | D |
| K438 | D | B | D |
| K439 | D | A | D |
| K440 | D | A | D |
| K441 | D | A | D |
| K442 | D | A | D |
| K443 | D | A | D |
| K444 | B | B | B |
| K445 | D | A | D |
| K446 | D | A | D |
| K447 | D | A | D |
| K448 | D | A | D |
| K449 | D | A | D |
| K450 | D | A | D |
| K451 | D | A | D |
| K452 | D | D | D |
| K453 | A | A | D |
| K454 | A | A | D |
| K455 | A | A | D |
| K456 | D | D | D |
| K457 | D | A | D |
| K458 | D | D | D |
| K459 | D | D | D |
| K460 | D | A | D |
| K461 | D | B | D |
| K462 | D | B | D |
| K463 | D | B | D |
| K464 | D | B | D |
| K465 | D | B | D |
| K466 | A | D | D |
| K467 | C | D | D |
| K468 | D | D | C |
| K479 | A | A | D |
| K480 | D | A | D |
| K481 | D | D | D |
| K482 | D | B | D |
| K483 | D | B | D |
| K484 | D | D | D |
| K485 | D | D | D |
| K486 | D | A | D |
| K487 | D | A | D |
| K488 | D | B | D |
| K489 | D | D | D |
| K490 | D | A | D |
| K491 | D | C | D |
| K492 | D | C | D |
| K493 | D | B | D |
| K494 | D | C | D |
| K495 | D | A | D |
| K496 | D | B | D |
| K497 | D | B | D |
| K498 | A | A | D |
| K499 | D | D | D |
| K500 | D | D | D |
| K501 | D | C | D |
| K502 | D | B | D |
| K503 | D | D | D |
| K504 | A | D | D |
| K505 | C | B | D |
| K506 | C | B | D |
| K507 | B | A | D |
| K508 | B | A | D |
| K509 | B | B | D |
| K510 | B | A | D |
| K511 | D | D | D |
| K512 | A | B | D |
| K513 | B | B | D |
| K514 | D | A | D |
| K515 | B | B | D |
| K516 | B | D | D |
| K517 | D | A | D |
| K518 | D | A | D |
| K519 | D | A | D |
| K520 | D | A | D |
| K521 | D | A | D |
| K522 | D | A | D |
| K523 | D | B | D |
| K524 | D | D | D |
| K525 | D | A | D |
| K526 | D | B | D |
| K527 | D | B | D |
| K528 | D | B | D |
| K529 | D | B | D |
| K530 | D | D | D |
| K531 | D | B | D |
| K532 | D | D | D |
| K533 | D | D | D |
| K534 | D | B | D |
| K535 | D | D | D |
| K536 | D | A | D |
| K537 | D | D | D |
| K538 | D | D | D |
| K539 | D | D | D |
| K540 | D | B | D |
| K541 | D | A | D |
| K542 | D | B | D |
| K543 | D | A | D |
| K544 | D | A | D |
| K545 | D | D | D |
| K546 | D | D | D |
| K547 | D | D | D |
| K548 | D | D | D |
| K549 | D | D | D |
| K550 | D | D | D |
| K551 | B | D | D |
| K552 | A | A | D |
| K553 | A | A | D |
| K554 | D | D | D |
| K555 | D | D | D |
| K556 | D | D | D |
| K557 | D | A | D |
| K558 | D | D | D |
| K559 | D | B | D |
| K560 | D | D | D |
| K561 | D | D | D |
| K562 | D | D | D |
| K563 | D | A | D |
| K564 | D | A | D |
| K565 | D | D | D |
| K566 | D | B | D |
| K567 | D | A | D |
| K568 | D | B | D |
| K569 | D | D | D |
| K570 | D | D | D |
| K571 | D | D | D |
| K572 | D | D | D |
| K573 | D | B | D |
| K574 | D | B | D |
| K575 | D | B | D |
| K576 | D | D | D |
| K577 | D | B | D |
| K578 | D | D | D |
| K579 | D | D | D |
| K580 | D | D | D |
| K581 | D | A | D |
| K582 | D | D | D |
| K583 | D | A | D |
| K584 | D | D | D |
| K585 | D | B | D |
| K586 | D | B | D |
| K587 | D | B | D |
| K588 | D | B | D |
| K589 | D | A | D |
| K590 | D | A | D |
| K591 | D | D | D |
| K592 | D | B | D |
| K593 | D | D | D |
| K594 | D | D | D |
| K595 | D | B | D |
| K596 | D | A | D |
| K597 | D | D | D |
| K598 | D | D | D |
| K599 | D | D | D |
| K600 | D | D | D |
| K601 | D | D | D |
| K602 | D | D | D |
| K603 | D | B | D |
| K604 | D | A | D |
| K605 | D | D | D |
| K606 | D | D | D |
| K607 | D | D | D |
| K608 | D | B | D |
| K609 | D | B | D |
| K610 | D | D | D |
| K611 | D | B | D |
| K612 | D | C | D |
| K613 | D | B | D |
| K614 | D | D | D |
| K615 | D | D | D |
| K616 | D | B | D |
| K617 | D | A | D |
| K618 | D | D | D |
| K619 | D | B | D |
| K620 | D | D | D |
| K621 | D | B | D |
| K622 | D | A | D |
| K623 | D | A | D |
| K624 | D | B | D |
| K625 | D | A | D |
| K626 | D | B | D |
| K627 | D | A | D |
| K628 | D | B | D |
| K629 | D | A | D |
| K630 | D | B | D |
| K631 | D | B | D |
| K632 | D | A | D |
| K633 | D | A | D |
| K634 | D | A | D |
| K635 | D | B | D |
| K636 | D | A | D |
| K637 | D | B | D |
| K638 | D | A | D |
| K639 | D | D | D |
| K640 | D | D | D |
| K641 | D | D | D |
| K642 | D | B | D |
| K643 | D | D | D |
| K644 | D | D | D |
| K645 | D | A | D |
| K646 | D | A | D |
| K647 | D | D | D |
| K648 | D | B | D |
| K649 | D | D | D |
| K650 | D | A | D |
| K651 | D | A | D |
| K652 | D | D | D |
| K653 | D | D | D |
| K654 | D | D | D |
| K655 | D | D | D |
| K656 | D | B | D |
| K657 | D | A | D |
| K658 | D | B | D |
| K659 | D | B | D |
| K660 | D | A | D |
| K661 | D | A | D |
| K662 | D | D | D |
| K700 | D | A | A |
| K701 | D | A | A |
| K702 | B | D | D |
| K703 | A | D | D |
| K704 | A | A | D |
| K705 | A | D | D |
| K706 | D | D | D |
| K707 | B | D | D |
| K708 | A | D | D |
| K709 | B | D | D |
| K710 | A | D | D |
| K711 | D | D | D |
| K712 | A | D | D |

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

What is claimed is:

1. A compound of Formula (II): 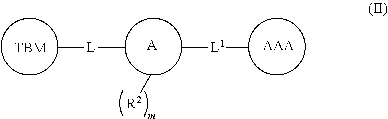
or a pharmaceutically acceptable salt thereof, wherein
TBM is a TYK binding moiety capable of binding to TYK2 protein;
L is a bivalent moiety that connects TBM to ring A and wherein:
Ring AAA is selected from: 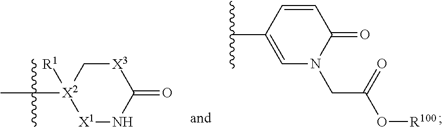
R¹⁰⁰ is C₁-C₆ alkyl or H;
X¹ is a bivalent moiety selected from a covalent bond, —CH₂—, —CHCF₃—, —SO₂—, —S(O)—, —P(O)R—, —
P(O)OR—, —P(O)NR₂—, —C(O)—, —C(S)—, or 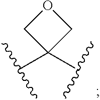
X² is a carbon atom, a nitrogen atom, or silicon atom;
X³ is a bivalent moiety selected from a covalent bond, —CR₂—, —NR—, —O—, —S—, or —Si(R)₂—;
R¹ is absent, hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R,
Ring A is selected from the group consisting of 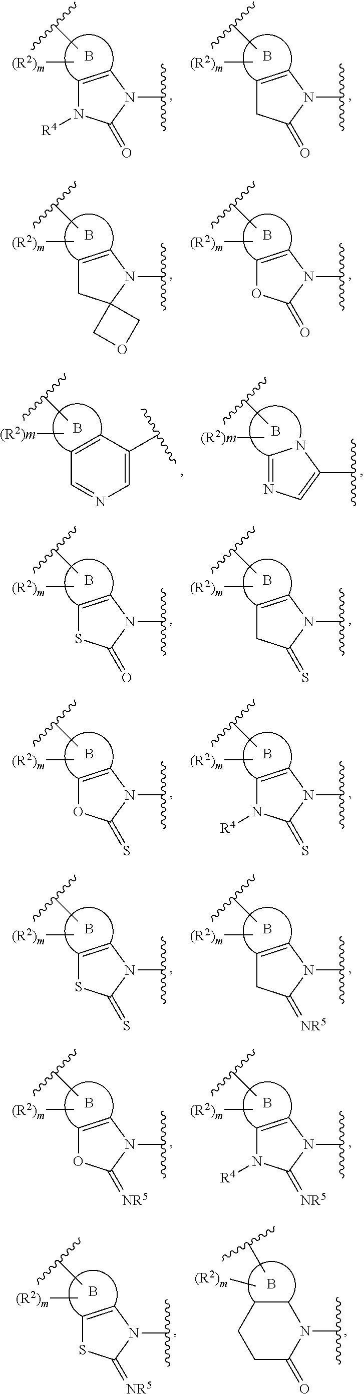 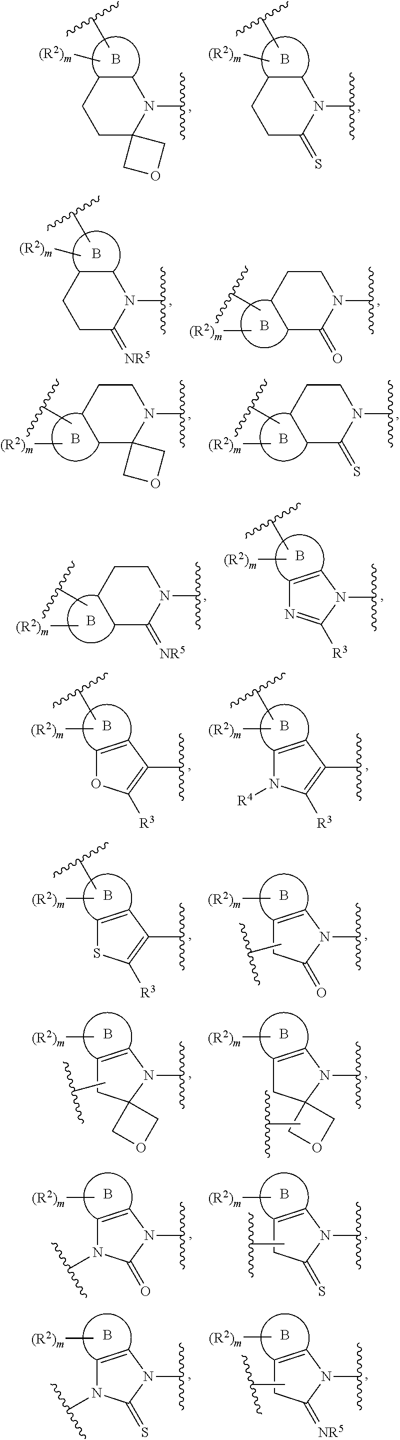 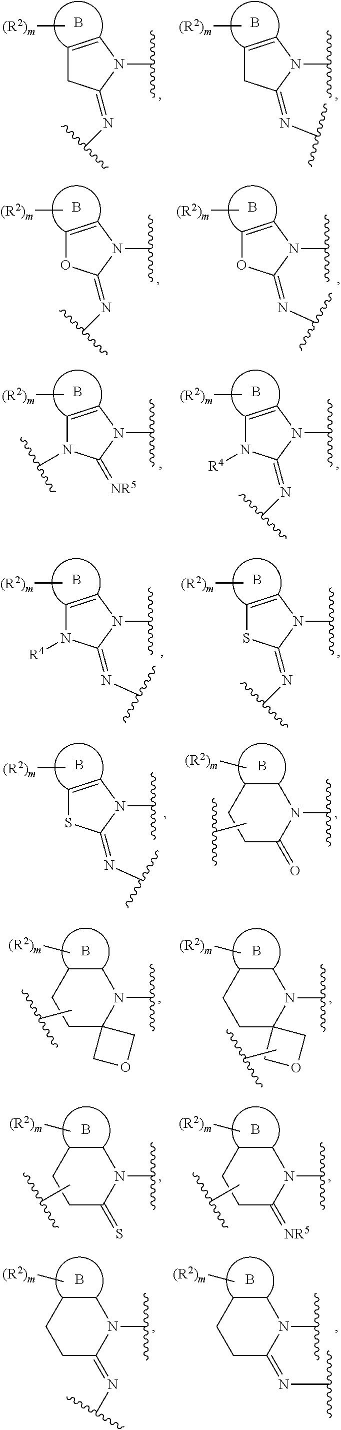 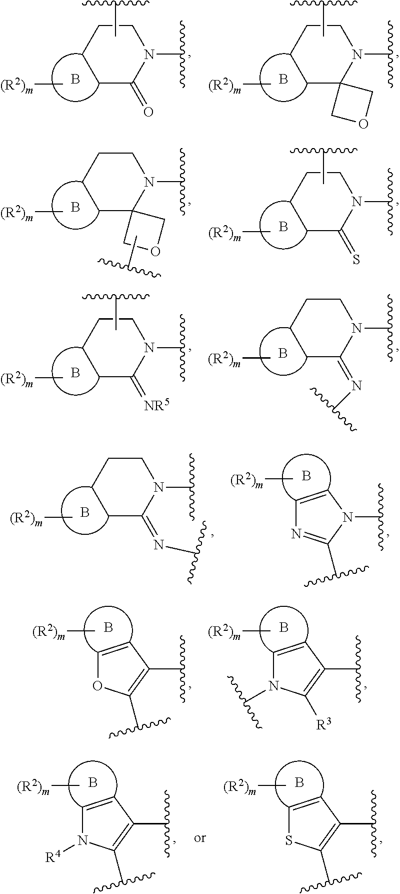
6-membered aryl optionally substituted with one or more occurrences of halogen, and null,
Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
R³ is selected from hydrogen, halogen, —OR, —N(R)₂, or —SR;
each R⁴ is independently hydrogen, —R⁶, halogen, —CN, —NO₂, —OR, —SR, —NR₂, —S(O)₂R, —S(O) 2NR2, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁-C₄ aliphatic, or —CN;
each R⁶ is independently an optionally substituted group selected from C1-6 aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L₁ is a covalent bond or a C₁-C₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
m is 0, 1, 2, 3 or 4; and
each R is independently hydrogen, or an optionally substituted group selected from C1-6 aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

2. The compound of claim 1, wherein ring AAA is:

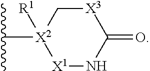

3. The compound of claim 1 or 2, wherein ring A is:

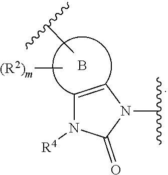

4. The compound of any one of claims 1-3, wherein ring A is:

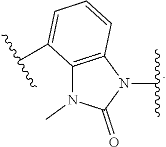 [SMILES: *c1cccc2c1n(C)c(=O)n2*]

5. The compound of claim 1 or 2, wherein ring A is: 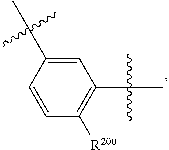
wherein R²⁰⁰ is halogen.

6. The compound of claim 1, wherein TBM has a structure of Formula (IIAA′): 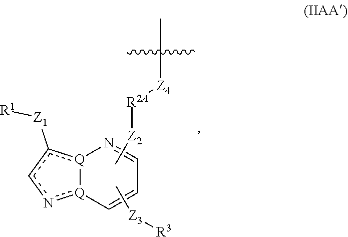
or a pharmaceutically acceptable salt thereof,
wherein Q is independently at each occurrence selected from —CH— and —N— when Q is attached to only single bonds, or Q is —C═ when Q is attached to a double bond;
R¹ is selected from a hydrogen, a C₁-C₆ aliphatic, Ring 1, —C₁-C₆ alkylene-Ring 1, and -Ring 1′-C₁-C₆ aliphatic; wherein each of the C₁-C₆ aliphatic, the Ring 1, the C₁-C₆ alkylene-Ring 1, and the -Ring 1′-C₁-C₆ aliphatic is independently optionally substituted with one or more of —F, —Cl, —Br, —I, and —OR^{C};
R^{2A} is selected from a covalent bond, a C₁-C₁₂ alkylene, which C₁-C₁₂ alkylene optionally comprises 1-8 heteroatoms independently selected from O, N, and S, and Ring 2A, or R^{2A} is —C₁-C₁₂ alkylene-Ring 2A-, -Ring 2A-Z₅-, or -(Ring 2A)-Z₅-(Ring 2A)-, wherein when R^{2A} is -(Ring 2A)-Z₅-(Ring 2A)- two Ring 2A may be the same or different; wherein each of the C₁-C₁₂ alkylene and the Ring 2A is independently optionally substituted with one or more of R^{K};
R³ is selected from a hydrogen and a C₁-C₆ aliphatic, which C₁-C₆ aliphatic optionally comprises 1-8 heteroatoms independently selected from O, N, and S, wherein the C₁-C₆ aliphatic is optionally substituted with one or more of R^{K};
Z₁ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₂ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₃ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₄ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₅ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S;
Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S;
Ring 2A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
each R^{K} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, a C₁-C₆ aliphatic group, or two R^{K} groups attached to the same carbon or sulfur atom are optionally taken together to form ═O;
each R^{C} is independently hydrogen or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S,
and 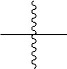
represents the point of attachment to L.

7. The compound of any one of claims 1-6, wherein TBM has a structure of Formula (IIAA′): 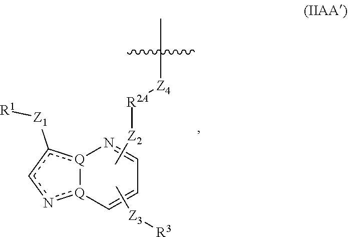
or a pharmaceutically acceptable salt thereof,
wherein Q is independently at each occurrence selected from —CH— and —N— when Q is attached to only single bonds, or Q is —C═ when Q is attached to a double bond;
R¹ is selected from a hydrogen, a C₁-C₆ aliphatic, Ring 1, —C₁-C₆ alkylene-Ring 1, and -Ring 1′-C₁-C₆ aliphatic; wherein each of the C₁-C₆ aliphatic, the Ring 1, the C₁-C₆ alkylene-Ring 1, and the -Ring 1′-C₁-C₆ aliphatic is independently optionally substituted with one or more of —CN, —F, —Cl, —Br, —I, and —OR^{C};
R^{2A} is selected from a covalent bond, a C₁-C₁₂ alkylene, which C₁-C₁₂ alkylene optionally comprises 1-8 heteroatoms independently selected from O, N, and S, and Ring 2A, or R^{2A} is —C₁-C₁₂ alkylene-Ring 2A-, -Ring 2A-Z₅-, or -(Ring 2A)-Z₅-(Ring 2A)-, wherein when R^{2A} is -(Ring 2A)-Z₅-(Ring 2A)- two Ring 2A may be the same or different; wherein each of the C₁-C₁₂ alkylene and the Ring 2A is independently optionally substituted with one or more of R^{K};
R³ is selected from a hydrogen and a C₁-C₆ aliphatic, which C₁-C₆ aliphatic optionally comprises 1-8 heteroatoms independently selected from O, N, and S, wherein the C₁-C₆ aliphatic is optionally substituted with one or more of R^{K};
Z₁ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₂ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₃ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₄ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₅ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 6-10 membered bicyclic saturated or partially unsaturated heterocyclyl having 1-2 heteroatoms independently selected from N, O, and S, and a 5-6 membered heteroaryl having 1-4 heteroatoms independently selected from N, O, and S;
Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S;
Ring 2A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
each R^{K} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, a C₁-C₆ aliphatic group, or two R^{K} groups attached to the same carbon or sulfur atom are optionally taken together to form ═O;
each R^{C} is independently hydrogen or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S,
and 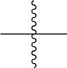
represents the point of attachment to L.

8. The compound of any one of claims 1-7, wherein TBM has a structure of Formula (IIAA′): 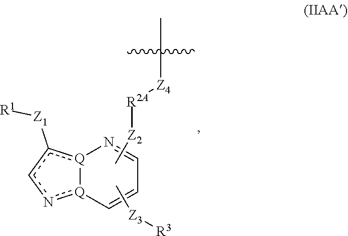
or a pharmaceutically acceptable salt thereof,
wherein Q^{A} is CH and Q^{B} is N or Q^{A} is N and Q^{B} is CH;
R¹ is selected from a hydrogen, a C₁-C₆ aliphatic, Ring 1, —C₁-C₆ alkylene-Ring 1, and -Ring 1′-C₁-C₆ aliphatic; wherein each of the C₁-C₆ aliphatic, the Ring 1, the C₁-C₆ alkylene-Ring 1, and the -Ring 1′-C₁-C₆ aliphatic is independently optionally substituted with one or more of —CN, —F, —Cl, —Br, —I, and —OR^{C};
R^{2A} is selected from a covalent bond, a C₁-C₁₂ alkylene, which C₁-C₁₂ alkylene optionally comprises 1-8 heteroatoms independently selected from O, N, and S, and Ring 2A, or R^{2A} is —C₁-C₁₂ alkylene-Ring 2A-, -Ring 2A-Z₅-, or -(Ring 2A)-Z₅-(Ring 2A)-, wherein when R^{2A} is -(Ring 2A)-Z₅-(Ring 2A)- two Ring 2A may be the same or different; wherein each of the C₁-C₁₂ alkylene and the Ring 2A is independently optionally substituted with one or more of R^{K};
R³ is selected from a hydrogen and a C₁-C₆ aliphatic, which C₁-C₆ aliphatic optionally comprises 1-8 heteroatoms independently selected from O, N, and S, wherein the C₁-C₆ aliphatic is optionally substituted with one or more of R^{K};
Z₁ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₂ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₃ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₄ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₅ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 6-10 membered bicyclic saturated or partially unsaturated heterocyclyl having 1-2 heteroatoms independently selected from N, O, and S, and a 5-6 membered heteroaryl having 1-4 heteroatoms independently selected from N, O, and S;
Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S;
Ring 2A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
each R^{K} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, a C₁-C₆ aliphatic group, or two R^{K} groups attached to the same carbon or sulfur atom are optionally taken together to form ═O;
each R^{C} is independently hydrogen or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S,
and 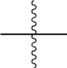
represents the point of attachment to L.

9. The compound of any one of claims 1-8, wherein TBM has a structure of Formula (IIA′), Formula (IIA″), or Formula (IIB): 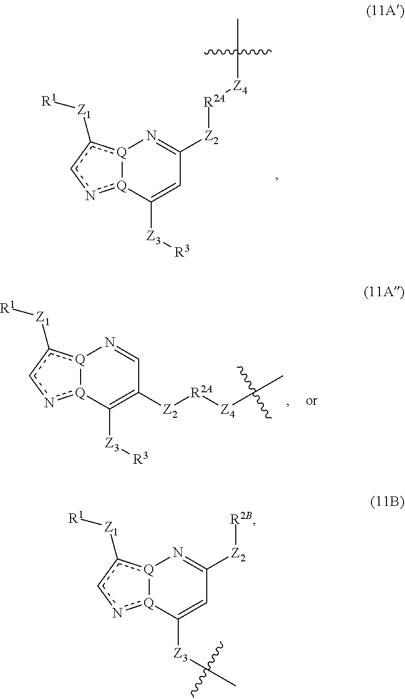
or a pharmaceutically acceptable salt thereof,
wherein Q is independently at each occurrence selected from —CH— and —N— when Q is attached to only single bonds, or Q is —C═ when Q is attached to a double bond;
R¹ is selected from a hydrogen, a C₁-C₆ aliphatic, Ring 1, —C₁-C₆ alkylene-Ring 1, and -Ring 1′-C₁-C₆ aliphatic; wherein each of the C₁-C₆ aliphatic, the C₁-C₆ alkylene-Ring 1, and the -Ring 1′-C₁-C₆ aliphatic is independently optionally substituted with one or more of —F, —Cl, —Br, —I, and —OR^{C};
R^{2A} is selected from a covalent bond, a C₁-C₁₂ alkylene, which C₁-C₁₂ alkylene optionally comprises 1-8 heteroatoms independently selected from O, N, and S, and Ring 2A, or R^{2A} is —C₁-C₁₂ alkylene-Ring 2A-, -Ring 2A-Z₅-, or -(Ring 2A)-Z₅-(Ring 2A)-, wherein when R^{2A} is -(Ring 2A)-Z₅-(Ring 2A)- two Ring 2A may be the same or different; wherein each of the C₁-C₁₂ alkylene and the Ring 2A is independently optionally substituted with one or more of R^{K};
R^{2B} is selected from a hydrogen, a C₁-C₆ aliphatic, and Ring 2B, or R^{2B} is —C₁-C₆ alkylene-Ring 2B, -Ring 2B′-C₁-C₆ aliphatic, -Ring 2B′-Z₅-Ring 2B wherein each of the C₁-C₆ aliphatic, the C₁-C₆ alkylene, the Ring 2B, the Ring 2B′ is independently optionally substituted with one or more of R^{K};
R³ is selected from a hydrogen and a C₁-C₆ aliphatic, which C₁-C₆ aliphatic optionally comprises 1-8 heteroatoms independently selected from O, N, and S, wherein the C₁-C₆ aliphatic is optionally substituted with one or more of R^{K};
Z₁ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₂ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₃ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₄ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₅ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S;
Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S;
Ring 2A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
Ring 2B is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S; and
Ring 2B′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
each R^{K} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, a C₁-C₆ aliphatic group, or two R^{K} groups attached to the same carbon or sulfur atom are optionally taken together to form ═O;
each R^{C} is independently hydrogen or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S,
and 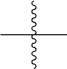
represents the point of attachment to L.

10. The compound of any one of claims 1-9, wherein TBM has a structure of Formula (IIA′), Formula (IIA″), or Formula (IIB): 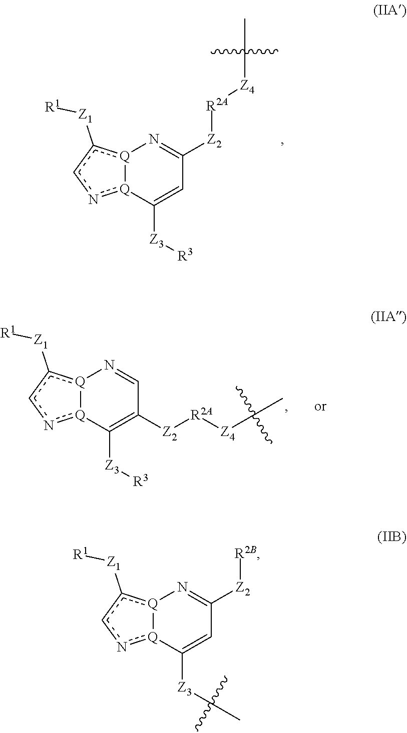
or a pharmaceutically acceptable salt thereof,
wherein Q is independently at each occurrence selected from —CH— and —N— when Q is attached to only single bonds, or Q is —C═ when Q is attached to a double bond;
R¹ is selected from a hydrogen, a C₁-C₆ aliphatic, Ring 1, —C₁-C₆ alkylene-Ring 1, and -Ring 1′-C₁-C₆ aliphatic; wherein each of the C₁-C₆ aliphatic, the C₁-C₆ alkylene-Ring 1, and the -Ring 1′-C₁-C₆ aliphatic is independently optionally substituted with one or more of —CN, —F, —Cl, —Br, —I, and —OR^{C};
R^{2A} is selected from a covalent bond, a C₁-C₁₂ alkylene, which C₁-C₁₂ alkylene optionally comprises 1-8 heteroatoms independently selected from O, N, and S, and Ring 2A, or R^{2A} is —C₁-C₁₂ alkylene-Ring 2A-, -Ring 2A-Z₅-, or -(Ring 2A)-Z₅-(Ring 2A)-, wherein when R^{2A} is -(Ring 2A)-Z₅-(Ring 2A)- two Ring 2A may be the same or different; wherein each of the C₁-C₁₂ alkylene and the Ring 2A is independently optionally substituted with one or more of R^{K};
R^{2B} is selected from a hydrogen, a C₁-C₆ aliphatic, and Ring 2B, or R^{2B} is —C₁-C₆ alkylene-Ring 2B, -Ring 2B′-C₁-C₆ aliphatic, -Ring 2B′-Z₅-Ring 2B wherein each of the C₁-C₆ aliphatic, the C₁-C₆ alkylene, the Ring 2B, the Ring 2B′ is independently optionally substituted with one or more of R^{K};
R³ is selected from a hydrogen and a C₁-C₆ aliphatic, which C₁-C₆ aliphatic optionally comprises 1-8 heteroatoms independently selected from O, N, and S, wherein the C₁-C₆ aliphatic is optionally substituted with one or more of R^{K};
Z₁ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₂ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₃ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₄ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₅ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 6-10 membered bicyclic saturated or partially unsaturated heterocyclyl having 1-2 heteroatoms independently selected from N, O, and S, and a 5-6 membered heteroaryl having 1-4 heteroatoms independently selected from N, O, and S;
Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S;
Ring 2A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
Ring 2B is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S; and
Ring 2B′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
each R^{K} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic) 3+, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, a C₁-C₆ aliphatic group, or two R^{K} groups attached to the same carbon or sulfur atom are optionally taken together to form ═O;
each R^{C} is independently hydrogen or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S,
and 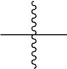
represents the point of attachment to L.

11. The compound of any one of claims 1-10, wherein TBM has a structure of Formula (IIA′-1-1), Formula (IIA″-1-1), or Formula (IIB-1-1): 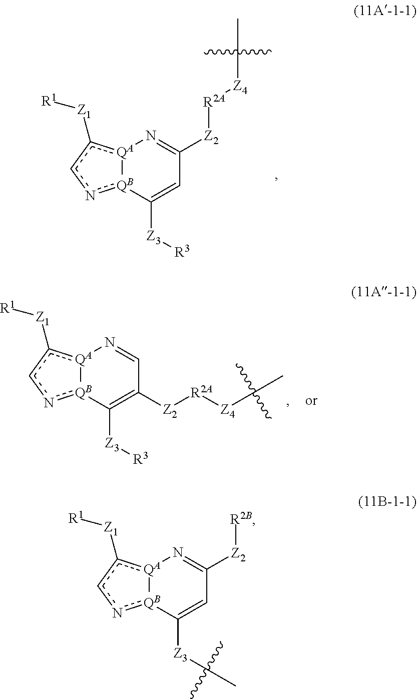
or a pharmaceutically acceptable salt thereof,
wherein Q^{A} is CH and Q^{B} is N or Q^{A} is N and Q^{B} is CH;
R¹ is selected from a hydrogen, a C₁-C₆ aliphatic, Ring 1, —C₁-C₆ alkylene-Ring 1, and -Ring 1′-C₁-C₆ aliphatic; wherein each of the C₁-C₆ aliphatic, the C₁-C₆ alkylene-Ring 1, and the -Ring 1′-C₁-C₆ aliphatic is independently optionally substituted with one or more of —CN, —F, —Cl, —Br, —I, and —OR^{C};
R^{2A} is selected from a covalent bond, a C₁-C₁₂ alkylene, which C₁-C₁₂ alkylene optionally comprises 1-8 heteroatoms independently selected from O, N, and S, and Ring 2A, or R^{2A} is —C₁-C₁₂ alkylene-Ring 2A-, -Ring 2A-Z₅-, or -(Ring 2A)-Z₅-(Ring 2A)-, wherein when R^{2A} is -(Ring 2A)-Z₅-(Ring 2A)- two Ring 2A may be the same or different; wherein each of the C₁-C₁₂ alkylene and the Ring 2A is independently optionally substituted with one or more of R^{K};
R^{2B} is selected from a hydrogen, a C₁-C₆ aliphatic, and Ring 2B, or R^{2B} is —C₁-C₆ alkylene-Ring 2B, -Ring 2B′-C₁-C₆ aliphatic, -Ring 2B′-Z₅-Ring 2B wherein each of the C₁-C₆ aliphatic, the C₁-C₆ alkylene, the Ring 2B, the Ring 2B′ is independently optionally substituted with one or more of R^{K};
R³ is selected from a hydrogen and a C₁-C₆ aliphatic, which C₁-C₆ aliphatic optionally comprises 1-8 heteroatoms independently selected from O, N, and S, wherein the C₁-C₆ aliphatic is optionally substituted with one or more of R^{K};
Z₁ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₂ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₃ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₄ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z₅ is selected from a covalent bond, —O—, —NR^{C}—, —S—, —C(O)—, —C(S)—, —CR^{C}₂—, —OC(O)—, —C(O)O—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 6-10 membered bicyclic saturated or partially unsaturated heterocyclyl having 1-2 heteroatoms independently selected from N, O, and S, and a 5-6 membered heteroaryl having 1-4 heteroatoms independently selected from N, O, and S;
Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S;
Ring 2A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
Ring 2B is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S; and
Ring 2B′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
each R^{K} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, a C₁-C₆ aliphatic group, or two R^{K} groups attached to the same carbon or sulfur atom are optionally taken together to form ═O;
each R^{C} is independently hydrogen or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S,
and 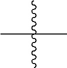
represents the point of attachment to L.

12. The compound of any one of claims 1-11, wherein TBM has a structure of Formula (IIBB′-4), Formula (IIBB′-4-1), Formula (IIA′A′-4), or Formula (IIA′A′-4-1): 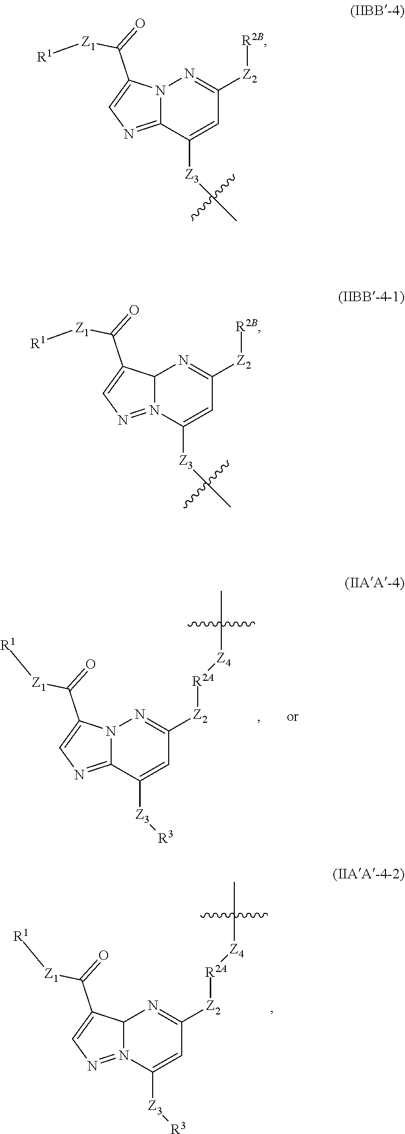
wherein R¹ is selected from a hydrogen, Ring 1, -Ring 1′-C₁-C₆ aliphatic, wherein the Ring 1 or the -Ring 1′-C₁-C₆ aliphatic is optionally substituted with one or more of —F, —Cl, —Br, —I, and —OR^{C};
Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S;
Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S;
R^{2A} is selected from a covalent bond, a C₁-C₁₂ alkylene, which C₁-C₁₂ alkylene optionally comprises 1-8 heteroatoms independently selected from O, N, and S, and Ring 2A, wherein each of the C₁-C₁₂ alkylene and the Ring 2A is optionally substituted with one or more of R^{K};
R^{2B} is selected from a hydrogen, a C₁-C₆ aliphatic, and Ring 2B, wherein the C₁-C₆ aliphatic and the Ring 2B is optionally substituted with one or more of R^{K};
Ring 2B is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
R³ is hydrogen or a C₁-C₃ aliphatic;
Z₁ is selected from a covalent bond, —O—, —NR^{C}—, —C(O)—, —CR^{C}₂—, —OC(O)—, —C(O)O—, —N(R^{C})C(O)—, and —C(O)N(R^{C})—;
Z₂ is selected from a covalent bond, —O—, —NR^{C}—, —C(O)—, —CR^{C}₂—, —OC(O)—, —C(O)O—, —N(R^{C})C(O)—, and —C(O)N(R^{C})—;
Z₃ is selected from a covalent bond, —O—, —NR^{C}—, —C(O)—, —CR^{C}₂—, —OC(O)—, —C(O)O—, —N(R^{C})C(O)—, and —C(O)N(R^{C})—;
Z₄ is selected from a covalent bond, —O—, —NR^{C}—, —C(O)—, —CR^{C}₂—, —OC(O)—, —C(O)O—, —N(R^{C})C(O)—, and —C(O)N(R^{C})—;
each R^{K} is independently hydrogen, —F, —Cl, —Br, —I, —OH, —O—(C₁-C₆ aliphatic), —NH₂, —NH—(C₁-C₆ aliphatic), —N(C₁-C₆ aliphatic)₂, —N(C₁-C₆ aliphatic)-OH, —N(C₁-C₆ aliphatic)-O—(C₁-C₆ aliphatic), —CN, —C(O)—C₁-C₆ aliphatic, —CHO, —CO₂H, —CO₂(C₁-C₆ aliphatic), —C(O)—NH₂, —C(O)—N(C₁-C₆ aliphatic)₂, —N(C₁-C₆ aliphatic)-C(O)—(C₁-C₆ aliphatic), —CF₃, —O—CF₃, a C₁-C₆ aliphatic group, or two R^{K} groups attached to the same carbon atom are optionally taken together to form ═O;
R^{C} is hydrogen or a C₁-C₆ aliphatic;
and 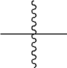
represents the point of attachment to L.

13. The compound of any one of claims 1-12, wherein TBM has a structure of Formula (IIBB′-3) or Formula (IIA′A′-3):

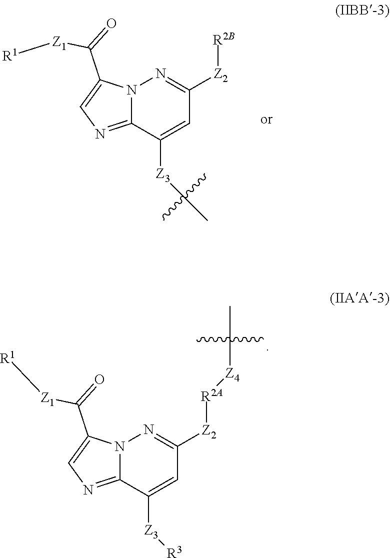

14. The compound of any one of claims 1-13, wherein TBM has a structure of Formula (IIBB′-2) or Formula (IIA′A′-2): 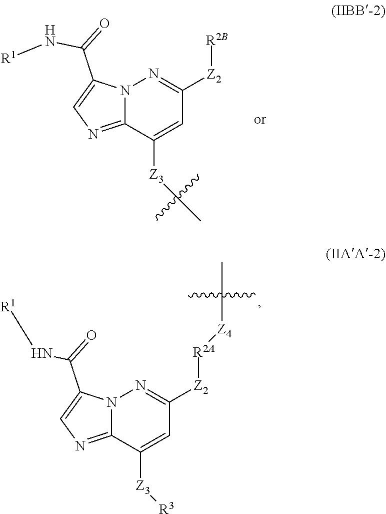
wherein R¹ is H, —C(CH₃)₂—CN,

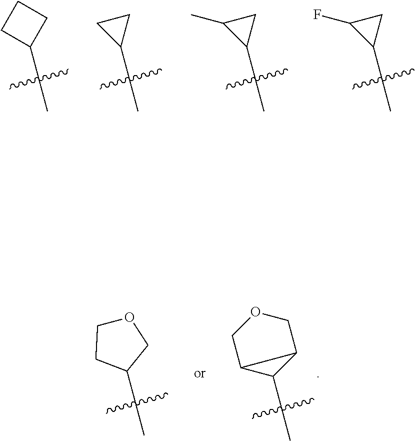

15. The compound of any one of claims 1-12, wherein the compound is represented by Formula (II-AB):

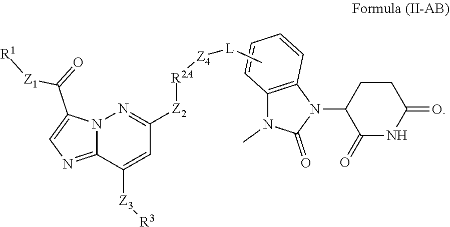

16. The compound of claim 15, wherein the compound is represented by Formula (II-AB-I):

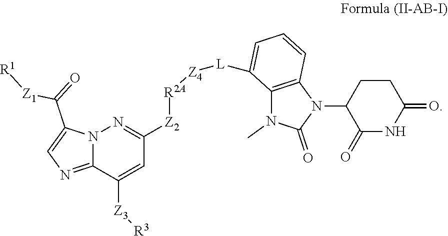

17. The compound of claim 15, wherein the compound is represented by Formula (II-AB-II):

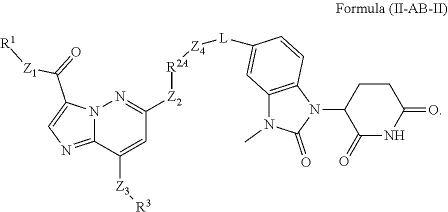

18. The compound of claim 15, wherein the compound is represented by Formula (II-AB-III):

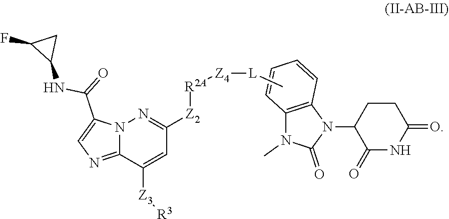

19. The compound of claim 18, wherein the compound is represented by Formula (II-AB-III):

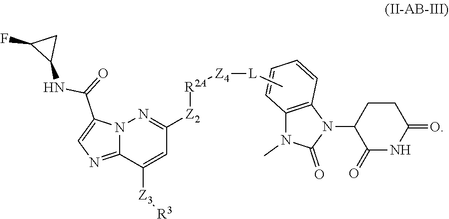

20. The compound of claim 18, wherein the compound is represented by Formula (II-AB-III):

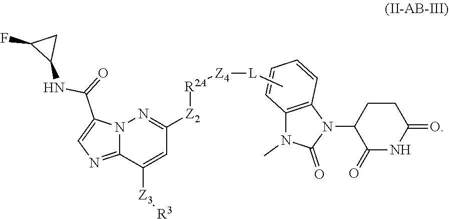

21. The compound of any one of claims 15-17, wherein R¹ is:
H, —C(CH₃)₂—CN,

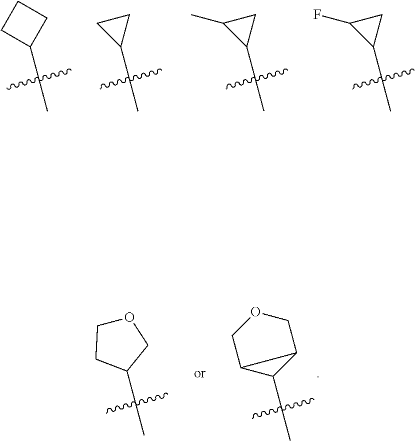

22. The compound of any one of claims 9-14, wherein R^{2B} is selected from a hydrogen, a C₁-C₆ aliphatic, and Ring 2B, wherein the C₁-C₆ aliphatic and the Ring 2B is optionally substituted with one or more of R^{K}.

23. The compound of any one of claims 9-14 and 22, wherein Ring 2B is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S.

24. The compound of any one of claims 6-23, wherein R¹ is selected from a hydrogen, Ring 1, -Ring 1′-C₁-C₆ aliphatic, wherein the Ring 1 or the -Ring 1′-C₁-C₆ aliphatic is optionally substituted with one or more of —F, —Cl, —Br, —I, and —OR^{C}.

25. The compound of any one of claims 6-24, wherein R^{2A} is selected from a covalent bond, a C₁-C₁₂ alkylene, which C₁-C₁₂ alkylene optionally comprises 1-8 heteroatoms independently selected from O, N, and S, and Ring 2A, wherein each of the C₁-C₁₂ alkylene and the Ring 2A is optionally substituted with one or more of R^{K}.

26. The compound of any one of claims 6-25, wherein R³ is selected from a hydrogen and a C₁-C₃ aliphatic, the C₁-C₃ aliphatic optionally comprises 1-8 heteroatoms independently selected from O, N, and S, and the C₁-C₃ aliphatic is optionally substituted with one or more of R^{K}.

27. The compound of any one of claims 6-26, wherein Z₁ is selected from a covalent bond, —O—, —NR^{C}—, —C(O)—, —CR^{C}₂—, —OC(O)—, —C(O)O—, —N(R^{C})C(O)—, and —C(O)N(R^{C})—.

28. The compound of any one of claims 6-26, wherein Z₂ is selected from a covalent bond, —O—, —NR^{C}—, —C(O)—, —CR^{C}₂—, —OC(O)—, —C(O)O—, —N(R^{C})C(O)—, and —C(O)N(R^{C})—.

29. The compound of any one of claims 6-28, wherein Z₂ is —NH—.

30. The compound of any one of claims 6-29, wherein Z₂ is a covalent bond.

31. The compound of any one of claims 1-30, wherein R^{C} is hydrogen or a C₁-C₆ aliphatic.

32. The compound of any one of claims 1-31, wherein R³ is an aliphatic C₁-C₄ hydrocarbon.

33. The compound of any one of claims 1-32, wherein R³ is —CH₃.

34. The compound of any one of claims 9-14 and 22-33, wherein the Z₂-R^{2B} group is selected from:

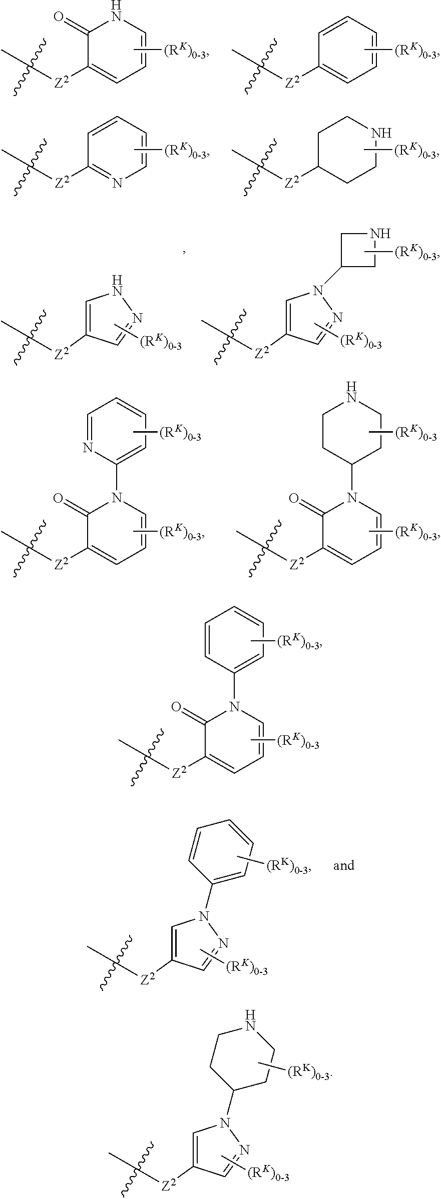

35. The compound of any one of claims 6-34, wherein Z₃ is selected from a covalent bond, —O—, —NR^{C}—, —C(O)—, —CR^{C}₂—, —OC(O)—, —C(O)O—, —N(R^{C})C(O)—, and —C(O)N(R^{C})—.

36. The compound of any one of claims 6-35, wherein Z₃ is —NH—.

37. The compound of any one of claims 6-36, wherein Z₄ is selected from a covalent bond, —O—, —NR^{C}—, or —C(O)—, CR^{C}₂, —OC(O)—, —C(O)O—, —N(R^{C})C(O)—, and —C(O)N(R^{C})—.

38. The compound of claim any one of claims 6-37, wherein Z₄ is a covalent bond, —O—, —C(O)—, —NH—, or —C(O)—N(CH₃).

39. The compound of any one of claims 6-38, wherein Z₅ is selected from a covalent bond, —O—, —NR^{C}—, —C(O)—, —CR^{C}₂—, —OC(O)—, —C(O)O—, —N(R^{C})C(O)—, and —C(O)N(R^{C})—.

40. The compound of claim 39, wherein Z₅ is a covalent bond or —O—.

41. The compound of any one of claims 6-40, wherein Ring 1 is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S.

42. The compound of any one of claims 6-41, wherein Ring 1′ is an optionally substituted ring selected from phenylenyl, a 3-7 membered saturated or partially unsaturated carbocyclylenyl, a 4-7 membered saturated or partially unsaturated heterocyclylenyl having 1-2 heteroatoms independently selected from N, O, and S, and a 5-6 membered heteroarylenyl having 1-4 heteroatoms independently selected from N, O, and S.

43. The compound of any one of claims 6-42, wherein Ring 2A is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-3 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof.

44. The compound of any one of claims 6-43, wherein each R^{K} is independently hydrogen, —F, —Cl, —Br, —I, —OH, —O—(C₁-C₆ aliphatic), —NH₂, —NH—(C₁-C₆ aliphatic), —N(C₁-C₆ aliphatic)₂, —N(C₁-C₆ aliphatic)-OH, —N(C₁-C₆ aliphatic)-O—(C₁-C₆ aliphatic), —CN, —C(O)—C₁-C₆ aliphatic, —CHO, —CO₂H, —CO₂(C₁-C₆ aliphatic), —C(O)—NH₂, —C(O)—N(C₁-C₆ aliphatic)₂, —N(C₁-C₆ aliphatic)-C(O)—(C₁-C₆ aliphatic), —CF₃, —O—CF₃, a C₁-C₆ aliphatic group, or two R^{K} groups attached to the same carbon atom are optionally taken together to form ═O.

45. The compound of any one of claims 6-44, wherein -Z₃- is covalent bond and R³ is hydrogen.

46. The compound of any one of claims 6-45, wherein -Z₃- is —NR^{C}— and R³ is —CH₃.

47. The compound of any one of claims 6-46, wherein TBM has a structure of Formula (IIBB′-1) or Formula (IIA′A′-1):

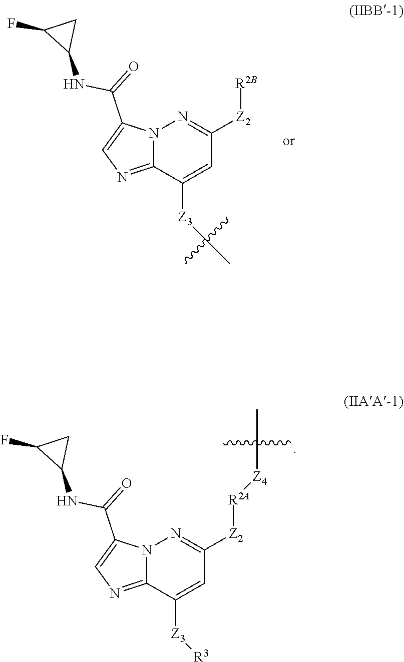

48. The compound of any one of claims 6-47, wherein Z₂ is —NH— and R^{2B} is Ring 2B.

49. The compound of any one of claims 6-48, wherein Ring 2B is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S.

50. The compound of any one of claims 6-49, wherein TBM has a structure of Formula (IIA′-2-1): 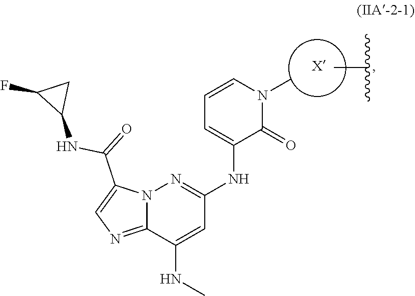
wherein Ring X′ is a ring selected from phenyl, a 4 to 7-membered saturated or partially unsaturated carbocyclyl or heterocyclyl with 1-3 heteroatoms independently selected from N, O, and S, and a 5 to 6-membered heteroaryl with 1-4 heteroatoms independently selected from N, O, and S, and each of which is optionally substituted with one or more of R^{K}.

51. The compound of claim 50, wherein TBM has a structure of Formula (IIA′-2-a):

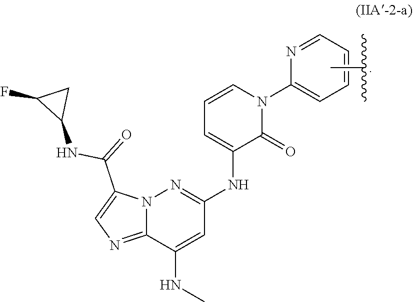

52. The compound of any one of claims 6-20, wherein TBM has a structure of Formula (IIA′-2-2): 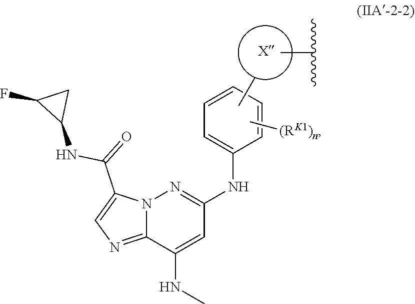
wherein
each R^{K1} is independently hydrogen, halo, C₁-C₆ aliphatic, halo substituted C₁-C₆ aliphatic, or —O—(C₁-C₆ aliphatic),
Ring X″ is a ring selected from phenyl, a 4 to 7-membered saturated or partially unsaturated carbocyclyl or heterocyclyl with 1-3 heteroatoms independently selected from N, O, and S, and a 5 to 6-membered heteroaryl with 1-4 heteroatoms independently selected from N, O, and S, and each of which is optionally substituted with one or more of R^{K}, and
n is 0-6.

53. The compound of claim 52, wherein TBM has a structure of Formula (IIA′-2-2a):

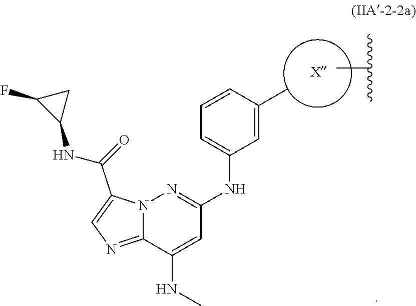

54. The compound of claim 53, wherein TBM has a structure of Formula (IIA′-2-b)

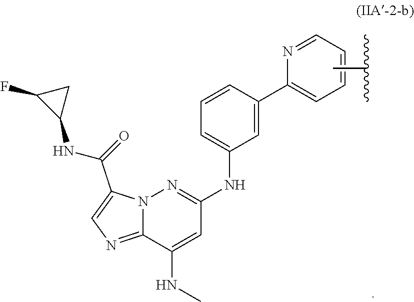

55. The compound of any one of claims 6-20, wherein TBM has a structure of Formula (IIA′-2-3): 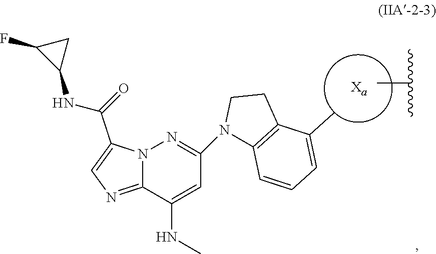
wherein Ring Xa is a ring selected from phenyl, a 4 to 7-membered saturated or partially unsaturated carbocyclyl or heterocyclyl with 1-3 heteroatoms independently selected from N, O, and S, and a 5 to 6-membered heteroaryl with 1-4 heteroatoms independently selected from N, O, and S, and each of which is optionally substituted with one or more of R^{K}.

56. The compound of claim 55, wherein TBM has a structure of Formula (IIA′-2-3a):

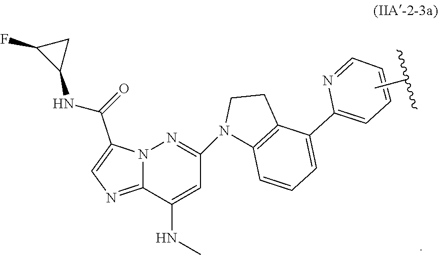

57. The compound of any one of claims 6-20, wherein TBM has a structure of Formula (IIA′-2-4) 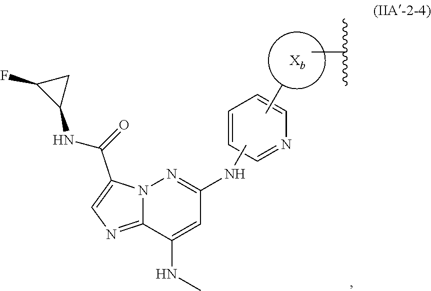
wherein Ring X_{b} is a ring selected from phenyl, a 4 to 7-membered saturated or partially unsaturated carbocyclyl or heterocyclyl with 1-3 heteroatoms independently selected from N, O, and S, and a 5 to 6-membered heteroaryl with 1-4 heteroatoms independently selected from N, O, and S, and each of which is optionally substituted with one or more of R^{K}.

58. The compound of any one of claims 6-20, wherein TBM has a structure of Formula (IIA′-2-1) 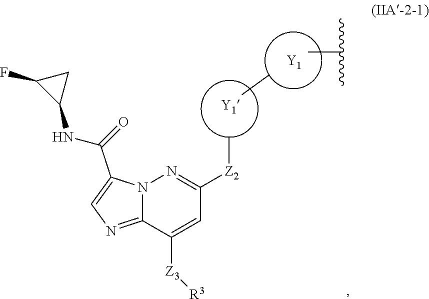
wherein Y1 and Y1′ are independently an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S.

59. The compound of any one of claims 1-5, wherein TBM has a structure of Formula (IIIAA) or Formula (IIIBB): 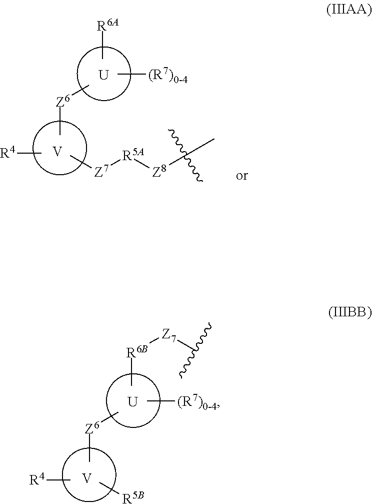
or a pharmaceutically acceptable salt thereof,
U is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
V is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
R⁴ is hydrogen, —F, —Cl, —Br, —I, —OH, —O—(C₁-C₆ aliphatic), —NH₂, —NH—(C₁-C₆ aliphatic), —N(C₁-C₆ aliphatic)₂, —CN, —C(O)—C₁-C₆ aliphatic, —CO₂H, —CO₂(C₁-C₆ aliphatic), —C(O)—NH₂, —C(O)—NH(C₁-C₆ aliphatic), —C(O)—N(C₁-C₆ aliphatic)₂, —C(O)—(C₁-C₆ aliphatic), —SO₂(C₁-C₆ aliphatic), —SO₂—N(C₁-C₆ aliphatic)₂, —S(O)—C₁-C₆ aliphatic, —CD₃, —CF₃, or —O—CF₃;
R^{5A} is selected from a covalent bond and Ring 5A, wherein the Ring 5A is optionally substituted with one or more R⁸;
R^{6A} is selected from a hydrogen, Ring 6A, and -Ring 6A′-Ring 6A, wherein the Ring 6A and the Ring 6A′ is independently optionally substituted with one or more R⁸;
R^{5B} is selected from a hydrogen, Ring 5B, and -Ring 5B′-Ring 5B, wherein the Ring 5B and the Ring 5B′ is independently optionally substituted with one or more R⁸;
R^{6B} is selected from a covalent bond and Ring 6B, wherein the Ring 6B is optionally substituted with one or more R⁸;
Ring 5A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
Ring 6A is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
Ring 6A′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S;
Ring 5B is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
Ring 5B′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S;
Ring 6B is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
Z⁶ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z⁷ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z⁸ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
each R⁷ is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic) 3+, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—NH(C₁-C₆ aliphatic), —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CD₃, —CF₃, or —O—CF₃;
each R⁸ is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—NH(C₁-C₆ aliphatic), —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CD₃, —CF₃, or —O—CF₃;
each R^{C} is independently hydrogen or an optionally substituted C₁-C₆ aliphatic group,
and 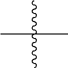
represents the point of attachment to L.

60. The compound of any one of claims 1-5, wherein TBM has a structure of Formula (IIIAA) or Formula (IIIBB): 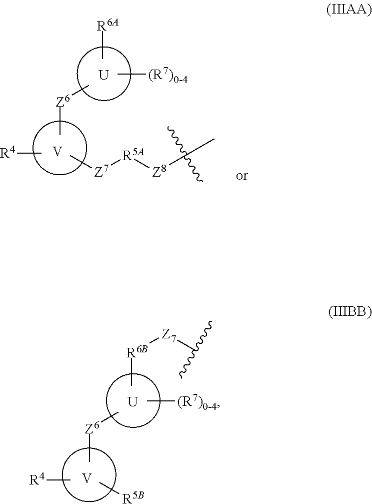
or a pharmaceutically acceptable salt thereof,
U is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
V is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
R⁴ is hydrogen, —F, —Cl, —Br, —I, —OH, —O—(C₁-C₆ aliphatic), —NH₂, —NH—(C₁-C₆ aliphatic), —N(C₁-C₆ aliphatic)₂, —CN, —C(O)—C₁-C₆ aliphatic, —CO₂H, —CO₂(C₁-C₆ aliphatic), —C(O)—NH₂, —C(O)—NH(C₁-C₆ aliphatic), —C(O)—N(C₁-C₆ aliphatic)₂, —C(O)—(C₁-C₆ aliphatic), —SO₂(C₁-C₆ aliphatic), —SO₂—N(C₁-C₆ aliphatic)₂, —S(O)—C₁-C₆ aliphatic, —CD₃, —CF₃, or —O—CF₃;
R^{5A} is selected from a covalent bond and Ring 5A, wherein the Ring 5A is optionally substituted with one or more R⁸;
R^{6A} is selected from a hydrogen, Ring 6A, and -Ring 6A′-Ring 6A, wherein the Ring 6A and the Ring 6A′ is independently optionally substituted with one or more R⁸;
R^{5B} is selected from a hydrogen, Ring 5B, and -Ring 5B′-Ring 5B, wherein the Ring 5B and the Ring 5B′ is independently optionally substituted with one or more R⁸;
R^{6B} is selected from a covalent bond and Ring 6B, wherein the Ring 6B is optionally substituted with one or more R⁸;
Ring 5A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
Ring 6A is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
Ring 6A′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S;
Ring 5B is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
Ring 5B′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S;
Ring 6B is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
Z⁶ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z⁷ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z⁸ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
each R⁷ is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic) 3+, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—NH(C₁-C₆ aliphatic), —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CD₃, —CF₃, or —O—CF₃;
each R⁸ is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃⁺, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—NH(C₁-C₆ aliphatic), —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CD₃, —CF₃, or —O—CF₃;
each R^{C} is independently hydrogen or an optionally substituted C₁-C₆ aliphatic group,
and 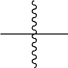
represents the point of attachment to L,
with the proviso that
(i) when TBM has a structure of Formula (IIIAA) and Z⁶ is —NH—, —Z⁶—U— is not 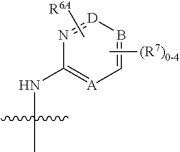
(ii) when TBM has a structure of Formula (IIIAA), R^{5A} is Ring 5A, and Z⁷ is —NH—, —Z⁶—V—Z⁷ is not 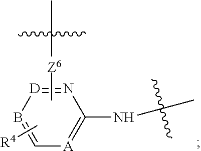
and
(iii) when TBM has a structure of Formula (IIIBB), Z⁶ is —NH—, —V—Z⁶— is not 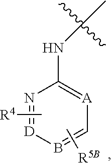
wherein A, B, D are independently —C═ or —NR^{AB}—,
R^{AB} is selected from hydrogen, halogen, hydroxyl, amino, cyano, nitro, CONR^{AB1}R^{AB2} optionally substituted C1-C8 aliphatic, and optionally substituted 3-10 membered carbocyclyl, wherein
R^{AB1} and R^{AB2} are independently selected from hydrogen, optionally substituted C₁-C₈ aliphatic, optionally substituted C₂-C₈ alkenyl, optionally substituted C₂-C₈ alkynyl, optionally substituted 3-10 membered carbocyclyl, and optionally substituted 4-10 membered heterocyclyl, or R^{AB1} and R^{AB2} together with the atom or atoms to which they are connected form a 3-20 membered carbocyclyl ring or 4-20 membered heterocyclyl ring.

61. The compounds of claim 59 or claim 60, wherein U is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, a 5-6 membered heteroaryl having 1-4 heteroatoms independently selected from N, O, and S.

62. The compounds of claim 59 or claim 60, wherein V is an optionally substituted ring selected from a 5-6 membered heteroaryl having 1-4 heteroatoms independently selected from N, O, and S, and an 8-10 membered bicyclic heteroaryl having 1-5 heteroatoms independently selected from N, O, and S.

63. The compound of claim 59 or claim 60, wherein TBM has a structure of Formula (IIIA) or Formula (IIIB): 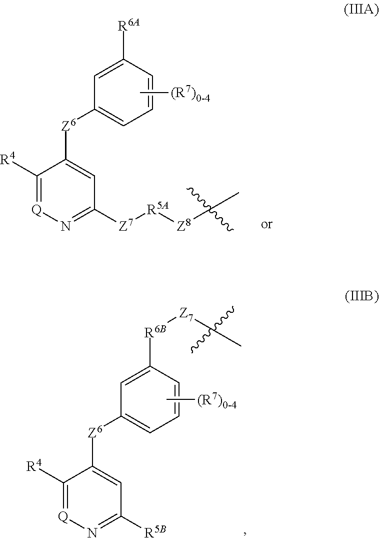
or a pharmaceutically acceptable salt thereof,
wherein Q′ is selected from —CH═ and —N═;
R⁴ is hydrogen, —F, —Cl, —Br, —I, —OH, —O—(C₁-C₆ aliphatic), —NH₂, —NH—(C₁-C₆ aliphatic), —N(C₁-C₆ aliphatic)₂, —CN, —C(O)—C₁-C₆ aliphatic, —CO₂H, —CO₂(C₁-C₆ aliphatic), —C(O)—NH₂, —C(O)—NH(C₁-C₆ aliphatic), —C(O)—N(C₁-C₆ aliphatic)₂, —C(O)—(C₁-C₆ aliphatic), —SO₂(C₁-C₆ aliphatic), —SO₂—N(C₁-C₆ aliphatic)₂, —S(O)—C₁-C₆ aliphatic, —CD₃, —CF₃, or —O—CF₃;
R^{5A} is selected from a covalent bond and Ring 5A, wherein the Ring 5A is optionally substituted with one or more R⁷;
R^{6A} is selected from a hydrogen, Ring 6A, and -Ring 6A′-Ring 6A, wherein the Ring 6A and the Ring 6A′ is independently optionally substituted with one or more R⁷;
R^{5B} is selected from a hydrogen, Ring 5B, and -Ring 5B′-Ring 5B, wherein the Ring 5B and the Ring 5B′ is independently optionally substituted with one or more R⁷;
R^{6B} is selected from a covalent bond and Ring 6B, wherein the Ring 6B is optionally substituted with one or more R⁷;
Ring 5A is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
Ring 6A is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
Ring 6A′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S;
Ring 5B is an optionally substituted ring selected from phenyl, an 8-10 membered bicyclic aryl, a 3-7 membered saturated or partially unsaturated carbocyclyl, an 8-10 membered bicyclic saturated or partially unsaturated carbocyclyl, a 4-7 membered saturated or partially unsaturated heterocyclyl having 1-2 heteroatoms independently selected from N, O, and S, an 8-10 membered bicyclic saturated or partially unsaturated heterocyclyl having 1-2 heteroatoms independently selected from N, O, and S, a 5-6 membered heteroaryl having 1-4 heteroatoms independently selected from N, O, and S, and an 8-10 membered bicyclic heteroaryl having 1-5 heteroatoms independently selected from N, O, and S;
Ring 5B′ is an optionally substituted ring selected from phenylenyl, an 8-10 membered bicyclic arylenyl, a 3-7 membered saturated or partially unsaturated carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S;
Ring 6B is an optionally substituted ring selected from phenylenyl, an 8-10 membered bicyclic arylenyl, a 3-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from N, O, and S, a 5-11 membered saturated or partially unsaturated spiro heterocyclylenyl, having 1-2 heteroatoms independently selected from N, O, and S, an 8-10 membered bicyclic saturated or partially unsaturated heterocyclylenyl having 1-2 heteroatoms independently selected from N, O, and S, a 5-6 membered heteroarylenyl having 1-4 heteroatoms independently selected from N, O, and S, and an 8-10 membered bicyclic heteroarylenyl having 1-5 heteroatoms independently selected from N, O, and S, or a combination of any two thereof;
Z⁶ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z⁷ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z⁸ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
each R⁷ is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic) 3+, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—NH(C₁-C₆ aliphatic), —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CD₃, —CF₃, or —O—CF₃;
each R^{C} is independently hydrogen or an optionally substituted C₁-C₆ aliphatic group,
and 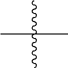
represents the point of attachment to L.

64. The compound of any one of claims 1-5, wherein TBM has a structure of Formula (IIIA-1), Formula (IIIB-1), Formula (IIIA-2), or Formula (IIIB-2): 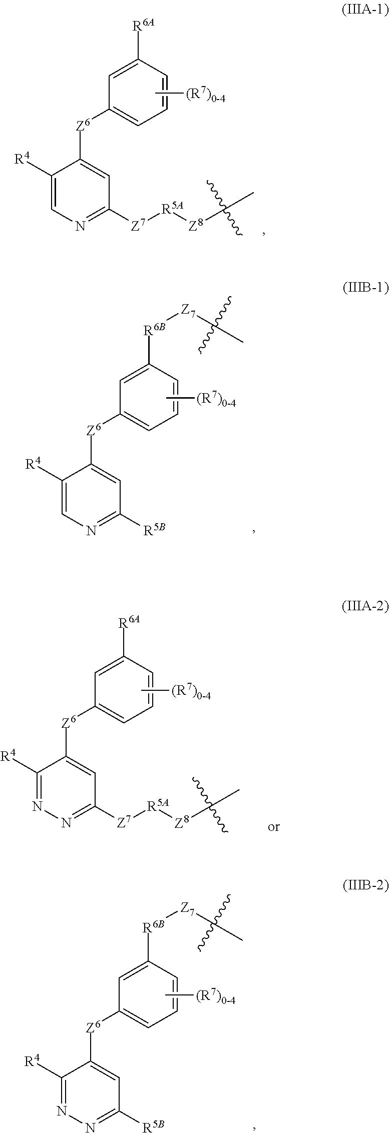
or a pharmaceutically acceptable salt thereof, wherein:
R⁴ is R⁴ is hydrogen, —F, —Cl, —Br, —I, —OH, —O—(C₁-C₆ aliphatic), —NH₂, —NH—(C₁-C₆ aliphatic), —N(C₁-C₆ aliphatic)₂, —CN, —C(O)—C₁-C₆ aliphatic, —CO₂H, —C(O)—NH₂, —C(O)—NH(C₁-C₆ aliphatic), —C(O)—N(C₁-C₆ aliphatic)₂, —C(O)—(C₁-C₆ aliphatic), —CD₃, —CF₃, or —O—CF₃;
R^{6A} is Ring 6A;
Ring 6A is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, wherein the Ring 6A is optionally substituted with one or more R⁸;
R^{6B} is Ring 6B;
Ring 6B is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, wherein the Ring 6B is optionally substituted with one or more R⁸;
each R⁷ is independently hydrogen, —F, —Cl, —Br, —I, —OH, or —O—(C₁-C₆ aliphatic);
each R⁸ is independently hydrogen, —F, —Cl, —Br, —I, or —OH;
Z⁶ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z⁷ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
Z⁸ is selected from a covalent bond, —O—, —NR^{C}—, —S—, —C(O)—, —C(S)—, —CR^{C}₂—, —OC(O)—, —C(O)O—, ——S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—;
each R^{C} is independently hydrogen or an optionally substituted C₁-C₆ aliphatic group,
and 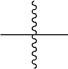
represents the point of attachment to L.

65. The compound of any one of claims 63-64, wherein TBM has a structure of Formula (IIIA-1) or Formula (IIIB-1): 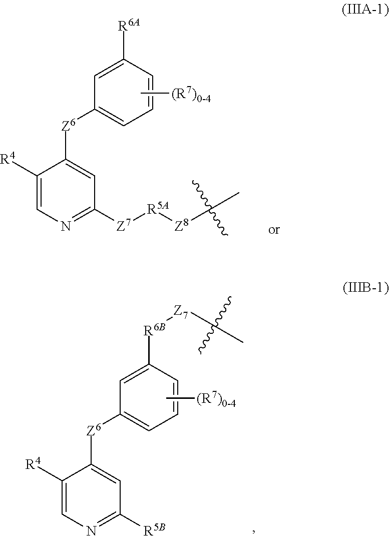
or a pharmaceutically acceptable salt thereof.

66. The compound of any one of claims 63-64, wherein TBM has a structure of Formula (IIIA-2) or Formula (IIIB-2): 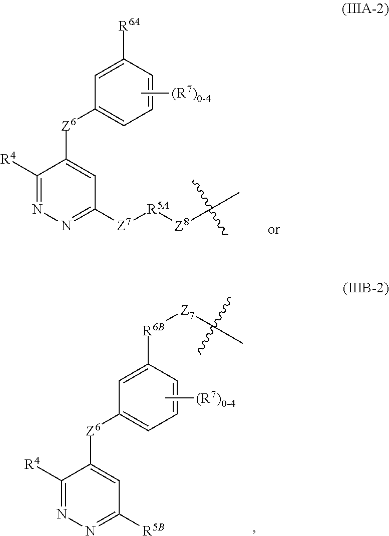
or a pharmaceutically acceptable salt thereof.

67. The compound of any one of claims 63-66 having the structure of Formula (IIIA), wherein R⁴ is hydrogen, —F, —Cl, —Br, —I, —OH, —O—(C₁-C₆ aliphatic), —NH₂, —NH—(C₁-C₆ aliphatic), —N(C₁-C₆ aliphatic)₂, —CN, —C(O)—C₁-C₆ aliphatic, —CO₂H, —C(O)—NH₂, —C(O)—NH(C₁-C₆ aliphatic), —C(O)—N(C₁-C₆ aliphatic)₂, —C(O)—(C₁-C₆ aliphatic), —CD₃, —CF₃, or —O—CF₃.

68. The compound of any one of claims 63-67 having the structure of Formula (IIIA), wherein R⁴ is hydrogen or —C(O)—NH(C₁-C₆ aliphatic).

69. The compound of any one of claims 63-68 having the structure of Formula (IIIA), wherein R⁴ is —C(O)—NH(CH₃).

70. The compound of any one of claims 63-68 having the structure of Formula (IIIA), wherein R^{6A} is Ring 6A.

71. The compound of claim 70, wherein Ring 6A is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S.

72. The compound of any one of claims 63-71 having the structure of Formula (IIIB), wherein R^{6B} is Ring 6B.

73. The compound of claim 72, wherein Ring 6B is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S.

74. The compound of any one of claims 63-73, wherein each R⁸ is independently hydrogen, —F, —Cl, —Br, —I, or —OH.

75. The compound of any one of claims 63-74, wherein at least one R⁸ is —F.

76. The compound of any one of claims 63-75 wherein each R⁷ is independently hydrogen, —F, —Cl, —Br, —I, —OH, or —O—(C₁-C₆ aliphatic).

77. The compound of any one of claims 63-76, wherein one R⁷ is —O—(C₁-C₆ aliphatic).

78. The compound of any one of claims 63-77 wherein Z⁶ is —NH—.

79. The compound of any one of claims 63-78, wherein TBM has a structure of Formula (IIIA-3-1) or Formula (IIIA-3-2): 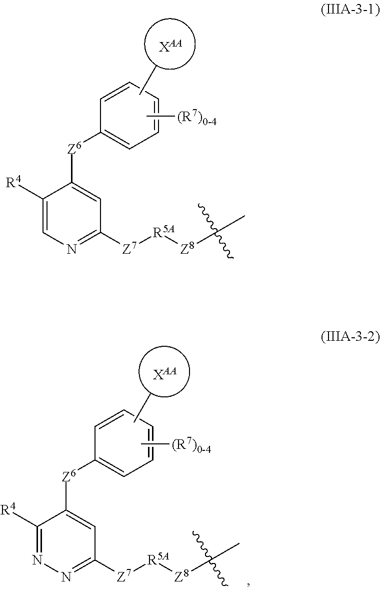
or a pharmaceutically acceptable salt thereof, wherein;
Ring X^{AA} is an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, or a 5-6 membered heteroaryl having 1-2 heteroatoms independently selected from N, O, and S.

80. The compound of any one of claims 63-79, wherein TBM has a structure of Formula (IIIA-3-1-1) or Formula (IIIA-3-2-1): 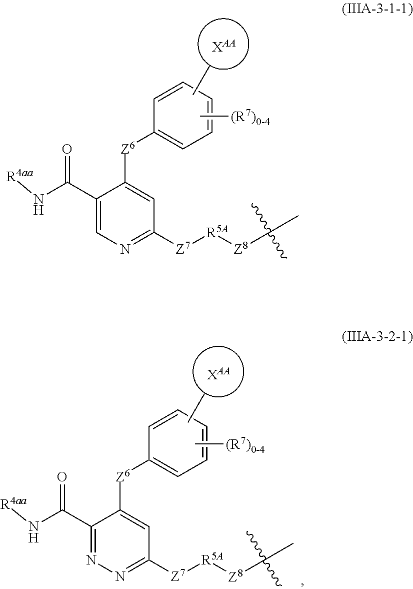
or a pharmaceutically acceptable salt thereof, wherein;
R^{4aa} is —CH₃, CD₃, —CH₂CH₃, or

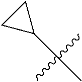 [SMILES: *C1CC1]

81. The compound of any one of claims 63-80, wherein TBM has a structure of Formula (IIIA-3-1-2) or Formula (IIIA-3-2-2): 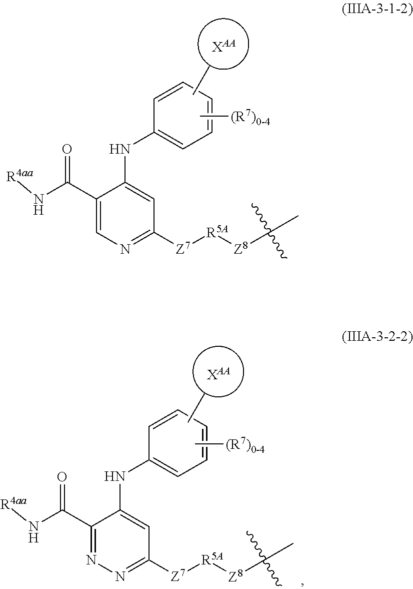
or a pharmaceutically acceptable salt thereof.

82. The compound of any one of claims 63-81, wherein TBM has a structure of Formula (IIIA-3): 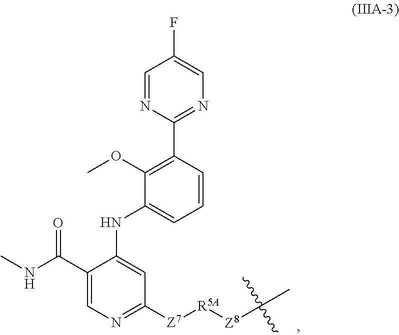
or a pharmaceutically acceptable salt thereof.

83. The compound of any one of claims 63-82, wherein Z⁷ is selected from a covalent bond, —NR^{C}—, —C(O)—, —NR^{C}C(O)—, —C(O)NR^{C}—, and —NR^{C}C(O)NR^{C}—.

84. The compound of any one of claims 63-83, wherein Z⁸ is selected from a covalent bond, —C(R^{C}₂)—, —NR^{C}—, —C(O)—, and —C(O)NR^{C}—.

85. The compound of any one of claims 63-84, wherein Z⁸ is —NH—.

86. The compound of any one of claims 63-85, wherein Z⁸ is selected from —C(O)NH— and —C(O)NCH₃—.

87. The compound of any one of claims 63-86 having the structure of Formula (IIIA), wherein R^{5A} is a covalent bond.

88. The compound of any one of claims 63-87 having the structure of Formula (IIIA), wherein R^{5A} is Ring 5A.

89. The compound of any one of claims 63-88 having the structure of Formula (IIIA), wherein R^{5A} is a 5-6 membered heteroarylenyl having 1-4 heteroatoms independently selected from N, O, and S.

90. The compound of any one of claims 63-89 having the structure of Formula (IIIA), wherein R^{5A} is

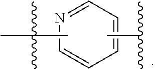

91. The compound of any one of claims 63-90, wherein TBM has a structure of Formula (IIIA-3-3) 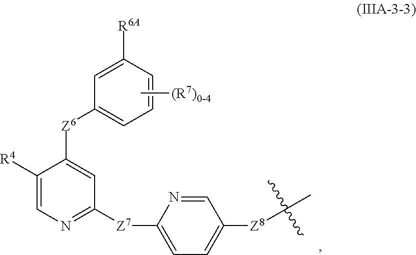
or a pharmaceutically acceptable salt thereof.

92. The compound of claim 91, wherein R^{6A} is hydrogen or an optionally substituted ring selected from phenyl, a 3-7 membered saturated or partially unsaturated carbocyclyl, a 4-7 membered saturated or partially unsaturated heterocyclyl having 1-2 heteroatoms independently selected from N, O, and S, or a 5-6 membered heteroaryl having 1-2 heteroatoms independently selected from N, O, and S.

93. The compound of any one of claims 91-92, wherein R⁷ is OCH₃.

94. The compound of any one of claims 91-93, wherein Z⁶ is —NH—.

95. The compound of any one of claims 91-94, wherein Z⁷ is —NH—.

96. The compound of any one of claims 63-64, wherein TBM is:

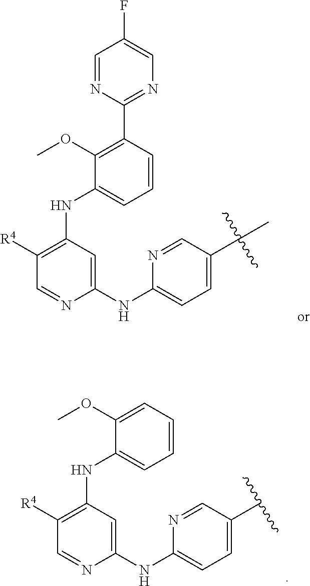

97. The compound of claim 96, wherein R⁴ is —C(O)—NH—CH₃, —C(O)—NH—CD₃, —C(O)—NH—CH₂CH₃, or

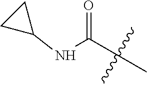 [SMILES: *C(=O)NC1CC1]

98. The compound of any one of claims 63-64, wherein TBM is:

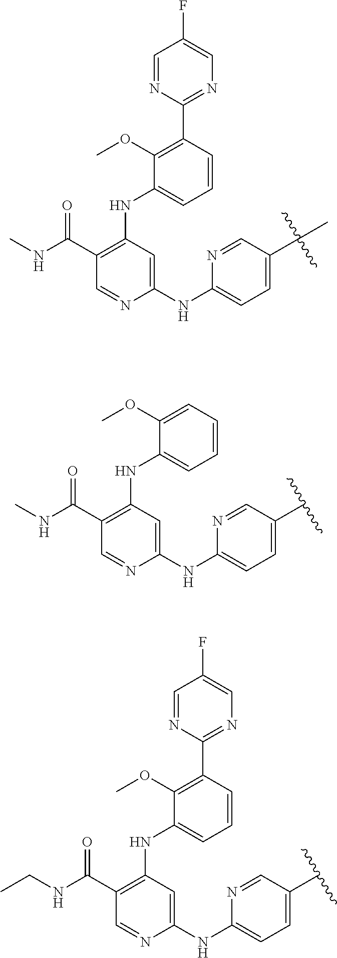 [SMILES: *c1ccc(Nc2cc(Nc3cccc(-c4ncc(F)cn4)c3OC)c(C(=O)NC)cn2)nc1 ; *c1ccc(Nc2cc(Nc3ccccc3OC)c(C(=O)NC)cn2)nc1 ; *c1ccc(Nc2cc(Nc3cccc(-c4ncc(F)cn4)c3OC)c(C(=O)NCC)cn2)nc1]

99. The compound of any one of claims 1-5, wherein the compound of Formula (I-b) has a structure of Formula (I-b-1) or Formula (I-b-2): 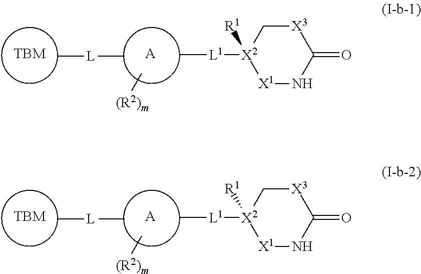
or a pharmaceutically acceptable salt thereof, wherein:
each of TBM, Ring A, L, L¹, R¹, R², X¹, X², X³, and m is as defined above.

100. The compound of any one of claims 1-5, wherein the compound of Formula (I-b) has a structure of Formula (I-b-3): 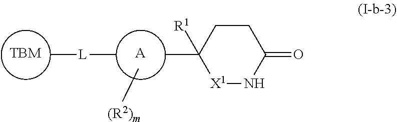
or a pharmaceutically acceptable salt thereof, wherein:
each of TBM, Ring A, L, R¹, R², X¹, and m is as defined above.

101. The compound of any one of claims 1-5, wherein the compound has an E3 ubiquitin ligase binding moiety as a cereblon E3 ubiquitin ligase binding moiety, and the compound has the structure of Formula (I-b-4): 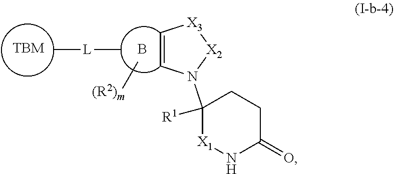
or a pharmaceutically acceptable salt thereof, wherein:
each of X₁, X₂, and X₃ is independently a covalent bond, —CH₂—, —C(O)—, —C(S)—, —NR— or 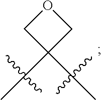 [SMILES: *C1(*)COC1]
R¹ is hydrogen, deuterium, halogen, —CN, —OR, —SR, —S(O)R, —S(O)₂R, —NR₂, or an optionally substituted C₁₋₄ aliphatic group;
each of R² is independently at each occurrence hydrogen, halogen, —CN, —NO₂, —OR, —SR,
—NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or
—N(R)S(O)₂R;
Ring B is a fused ring selected from a 6-membered aryl containing 0-2 nitrogen atoms, a 5 to 7-membered partially saturated carbocyclyl, a 5 to 7-membered partially saturated heterocyclyl having 1-2 heteroatoms independently selected from nitrogen, oxygen or sulfur, or a 5-membered heteroaryl having 1-3 heteroatoms independently selected from nitrogen, oxygen or sulfur;
m is an integer from 0 to 4;
each R is independently at each occurrence hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃+, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, and combinations thereof, or:
two R groups on the same nitrogen are optionally taken together with their intervening atoms to form a 4 to 7-membered saturated, partially unsaturated, or heteroaryl ring having 0-3 heteroatoms in addition to the nitrogen, independently selected from N, O, and S.

102. The compound of any one of claim 101, wherein X₁ and X₂ are —C(O)— and X₃ is —NR—, wherein R is hydrogen, or an optionally substituted C₁-C₆ aliphatic group.

103. The compound of any one of claims 101-102, wherein R¹ and R² are a hydrogen at each occurrence.

104. The compound of any one of claims 101-103, wherein Ring A is a fused phenyl ring.

105. The compound of any one of claims 101-104, having the structure selected from Formula (1-a-11), Formula (1-a′-11), and Formula (1-a″-11): 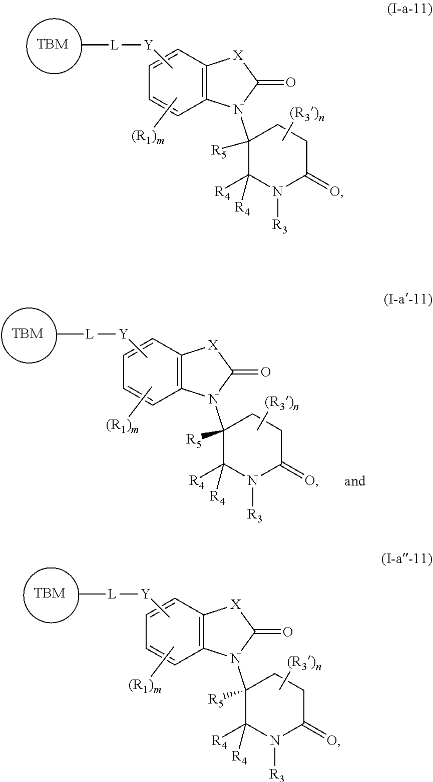
or a pharmaceutically acceptable salt thereof,
wherein L and TBM are as defined above, and wherein:
Y is a bond, Y₁, O, NH, NR₂, C(O)O, OC(O), C(O)NR₂′, NR₂′C(O), Y₁—O, Y₁—NH, Y₁—NR₂, Y₁—C(O), Y₁—C(O)O, Y₁—OC(O), Y₁—C(O)NR₂′, or Y₁—NR₂′C(O), wherein Y₁ is C₁-C₆ alkylene, C₂-C₆ alkenylene, or C₂-C₆ alkynylene;
X is C(O) or C(R₃)₂;
each R₁ is independently halogen, nitro, NH₂, OH, C(O)OH, C₁-C₆ alkyl, or C₁-C₆ alkoxy;
R₂ is C₁-C₆ alkyl, C₂-C₆ alkenyl, C₃-C₈ cycloalkyl, 3- to 8-membered heterocycloalkyl, C(O)—C₁-C₆ alkyl, C(O)—C₂-C₆ alkenyl, C(O)—C₃-C₈ cycloalkyl, or C(O)-3- to 8-membered heterocycloalkyl, and R₂ is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy;
R₂′ is H, C₁-C₆ alkyl, C₂-C₆ alkenyl, C₃-C₈ cycloalkyl, or 3- to 8-membered heterocycloalkyl, and R₂′, when not being H, is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy;
each R₃ is independently H or C₁-C₃ alkyl optionally substituted with C₆-C₁₀ aryl or 5- to 10-membered heteroaryl;
each R₃′ is independently C₁-C₃ alkyl;
each R₄ is independently H or C₁-C₃ alkyl; or two R₄, together with the carbon atom to which they are attached, form C(O), a C₃-C₆ carbocycle, or a 4-, 5-, or 6-membered heterocycle comprising 1 or 2 heteroatoms selected from N and O;
R₅ is H, C₁-C₃ alkyl, F, or Cl;
each Rₐ independently is H or C₁-C₆ alkyl;
R_{b} is H or tosyl;
t is 0 or 1;
m is 0, 1, 2 or 3; and
n is 0, 1 or 2.

106. The compound of any one of claims 101-105, wherein the E3 ubiquitin ligase binding moiety is

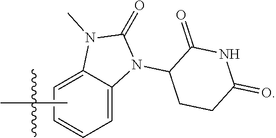

107. The compound of any one of claims 101-105, wherein the E3 ubiquitin ligase binding moiety is

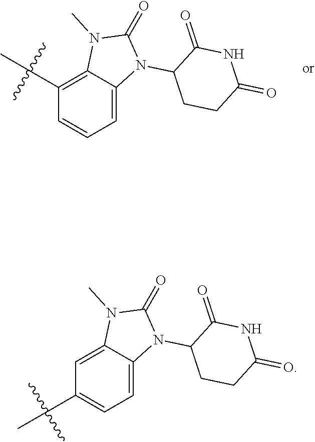 [SMILES: *c1cccc2c1n(C)c(=O)n2C1CCC(=O)NC1=O ; *c1ccc2c(c1)n(C)c(=O)n2C1CCC(=O)NC1=O]

108. The compound of any one of claims 63-107, wherein the compound has a structure of: 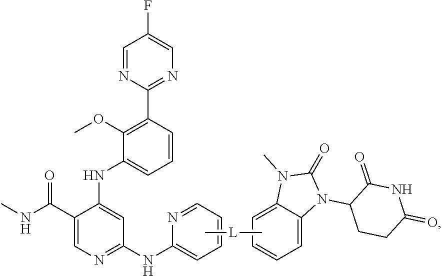
or a pharmaceutically acceptable salt thereof.

109. The compound of claim 108 having the structure selected from the group consisting of: 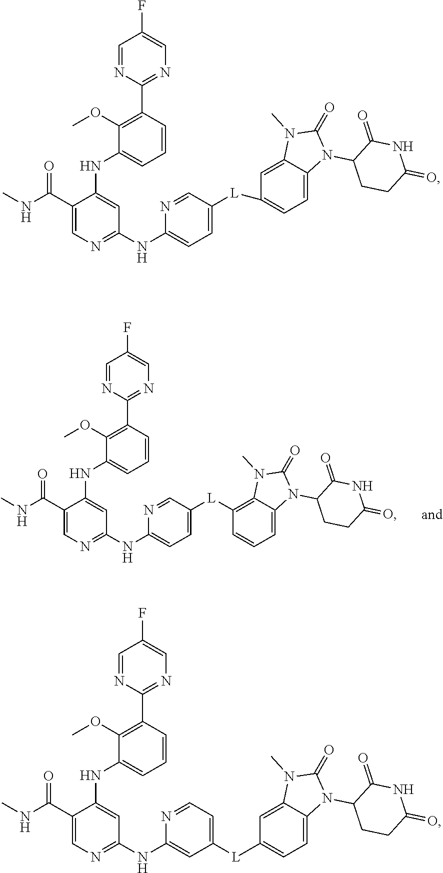
or a pharmaceutically acceptable salt thereof.

110. The compound of any one of claims 1-109, wherein L is a covalent bond or a bivalent, saturated or partially unsaturated, straight or branched C₁-C₂₀ alkylene chain, wherein 0-6 methylene units of L are independently replaced by -Cy-, —O—, —NR^{C}—, —S—, —OC(O)—, —C(O)O—, —C(O)—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—, and combinations thereof,
wherein -Cy- is independently at each occurrence an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl, having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur, and
each R^{C} is independently at each occurrence hydrogen, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, and combinations thereof.

111. The compound of any one of claims 1-110, wherein L is a covalent bond.

112. The compound of any one of claims 1-111 wherein L comprises a saturated straight C₁-C₁₂ alkylene chain.

113. The compound of any one of claims 1-112, wherein L comprises a saturated straight C₁-C₈ alkylene chain.

114. The compound of any one of claims 1-113, wherein L comprises a saturated straight C₂-C₆ alkylene chain.

115. The compound of any one of claims 1-114, wherein L comprises a saturated straight C₄ alkylene chain.

116. The compound of any one of claims 1-114, wherein 0-5 methylene units of L are independently replaced by -Cy-, —O—, —NR^{C}—, —N(R^{C})C(O)—, —C(O)N(R^{C})—, —OC(O)N(R^{C})—, and —N(R^{C})C(O)O—, and combinations thereof,

117. The compound of any one of claims 1-110, wherein L comprises a polyethylene glycol (PEG) ( 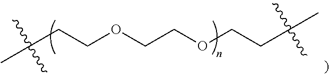
chain, wherein n is an integer from 1 to 10.

118. The compound of any one of claims 1-110, wherein L comprises at least one -Cy-.

119. The compound of claim 118, wherein -Cy- is each independently an optionally substituted bivalent ring selected from phenylenyl, a 4-6 membered saturated or partially unsaturated carbocyclylenyl, a 6-10 membered saturated or partially unsaturated spiro carbocyclylenyl, a 4-7 membered saturated or partially unsaturated heterocyclylenyl having 1-2 heteroatoms independently selected from nitrogen, oxygen, and sulfur, a 8-10 membered saturated or partially unsaturated spiro heterocyclylenyl, having 1-2 heteroatoms independently selected from nitrogen, oxygen, and sulfur, or a 5-6 membered heteroarylenyl having 1-4 heteroatoms independently selected from nitrogen, oxygen, and sulfur.

120. The compound of claim 119, wherein -Cy- is each independently an optionally substituted bivalent ring selected from phenylenyl, a 4-6 membered saturated or partially unsaturated carbocyclylenyl, a 6-10 membered saturated or partially unsaturated spiro carbocyclylenyl, a 4-7 membered saturated or partially unsaturated heterocyclylenyl having 1-2 nitrogen atoms, a 8-10 membered saturated or partially unsaturated spiro heterocyclylenyl, having 1-2 nitrogen atoms, or a 5-6 membered heteroarylenyl having 1-3 nitrogen atoms.

121. The compound of claim 230, wherein -Cy- is each independently an optionally substituted bivalent ring selected from phenylenyl, a 4 or 6 membered saturated or partially unsaturated carbocyclylenyl, a 9 membered saturated or partially unsaturated spiro carbocyclylenyl, a 4 or 6 membered saturated or partially unsaturated heterocyclylenyl having 1-2 nitrogen atoms, a 4 or 6 membered saturated or partially unsaturated spiro heterocyclylenyl, having 1-2 nitrogen atoms, or a 5 or 6 membered heteroarylenyl having 1-3 nitrogen atoms.

122. The compound of any one of claims 1-110 and 118, wherein L comprises a structure selected from the group consisting of: 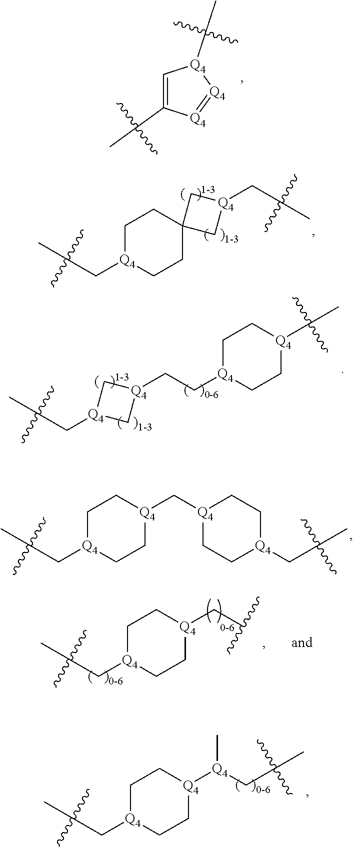
where each Q₄ is independently selected from —CH— and —N— when Q₄ is attached to only single bonds, or Q₄ is ═CH— or ═N— when Q₄ is attached to a double bond.

123. The compound of any one of claims 1-110, wherein L comprises a structure selected from the group consisting of:

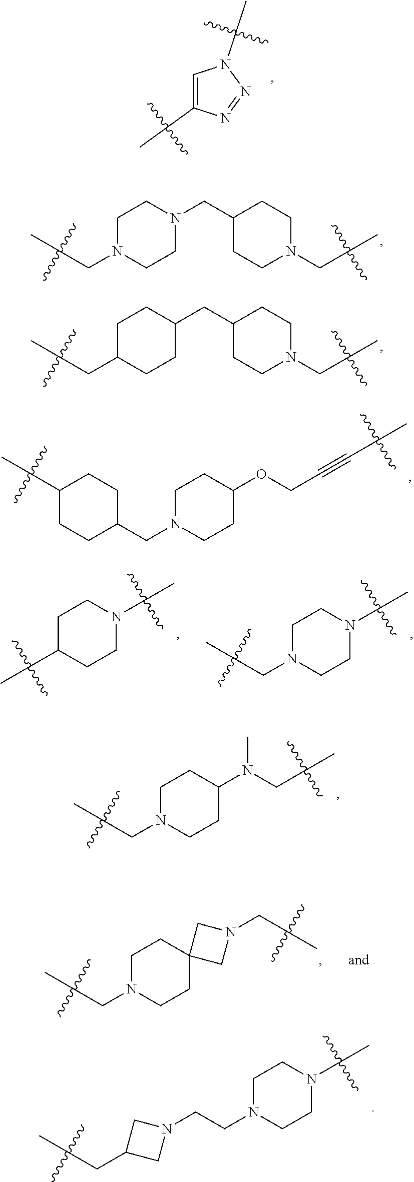

124. The compound of any one of claims 1-110, wherein L comprises a structure selected from the group consisting of:

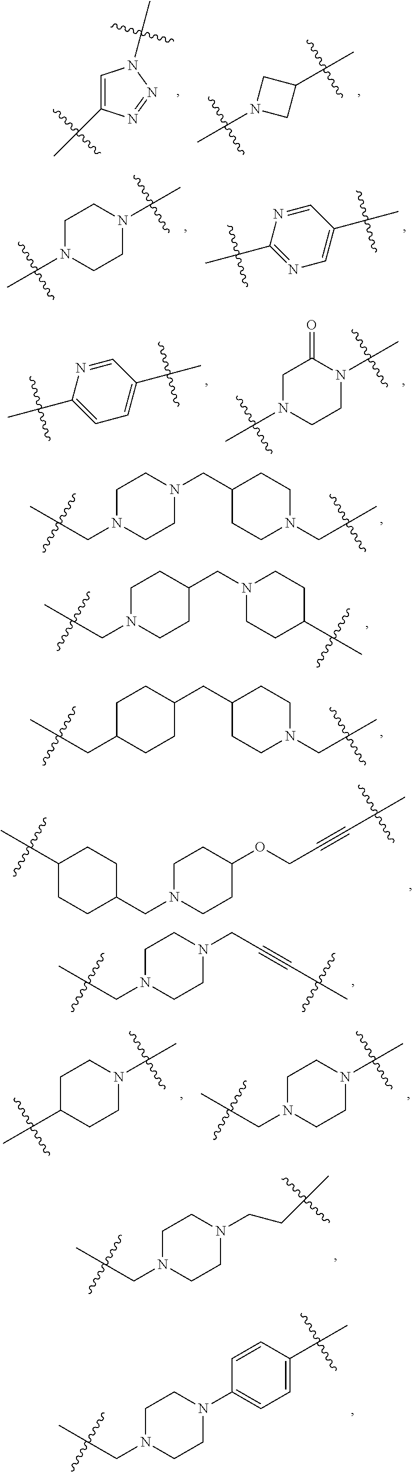

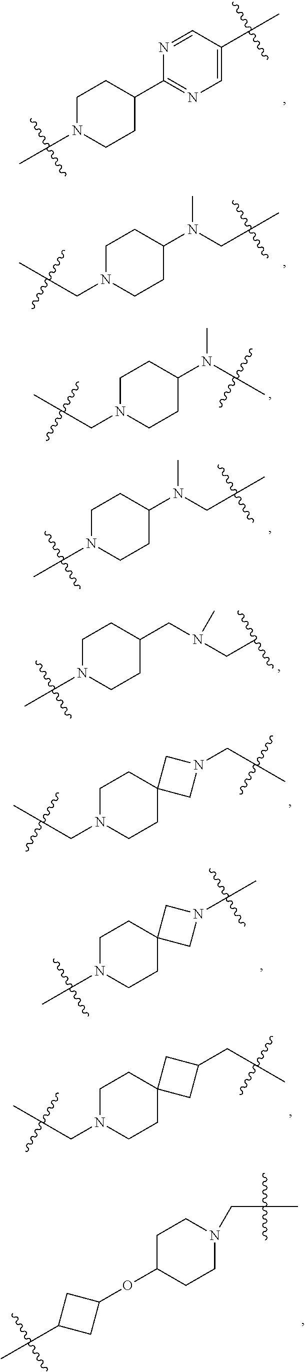

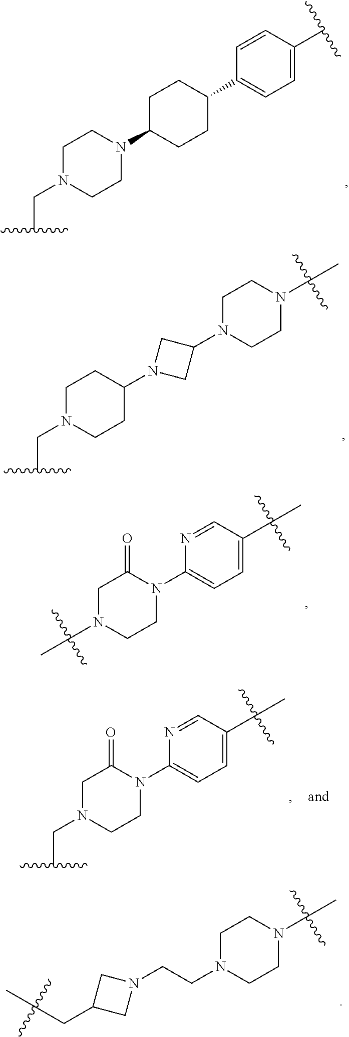 [SMILES: *CN1CCN([C@H]2CC[C@H](c3ccc(*)cc3)CC2)CC1 ; *CN1CCC(N2CC(N3CCN(*)CC3)C2)CC1 ; *c1ccc(N2CCN(*)CC2=O)nc1 ; *CN1CCN(c2ccc(*)cn2)C(=O)C1 ; *CC1CN(CCN2CCN(*)CC2)C1]

125. The compound of any one of claims 1-110, wherein L comprises a structure selected from the group consisting of:

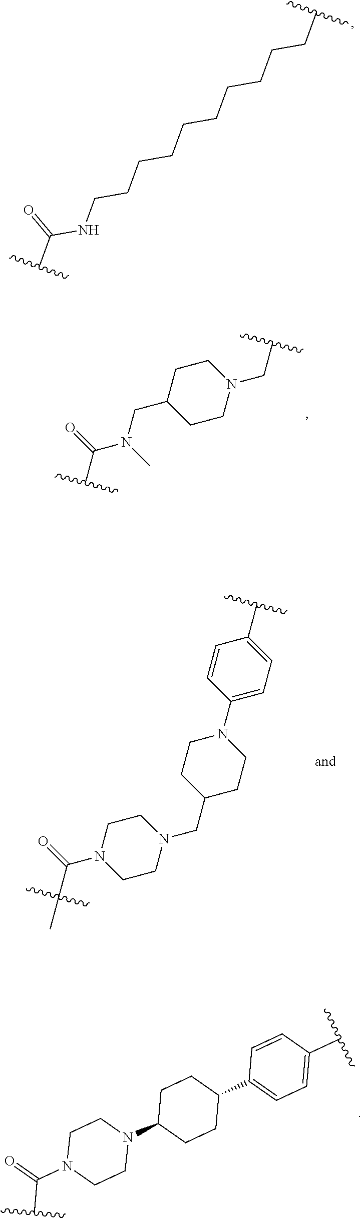 [SMILES: *CCCCCCCCCCNC(*)=O ; *CN1CCC(CN(C)C(*)=O)CC1 ; *C(=O)N1CCN(CC2CCN(c3ccc(*)cc3)CC2)CC1 ; *C(=O)N1CCN([C@H]2CC[C@H](c3ccc(*)cc3)CC2)CC1]

126. The compound of any one of claims 1-110, wherein L comprises a structure selected from the group consisting of:

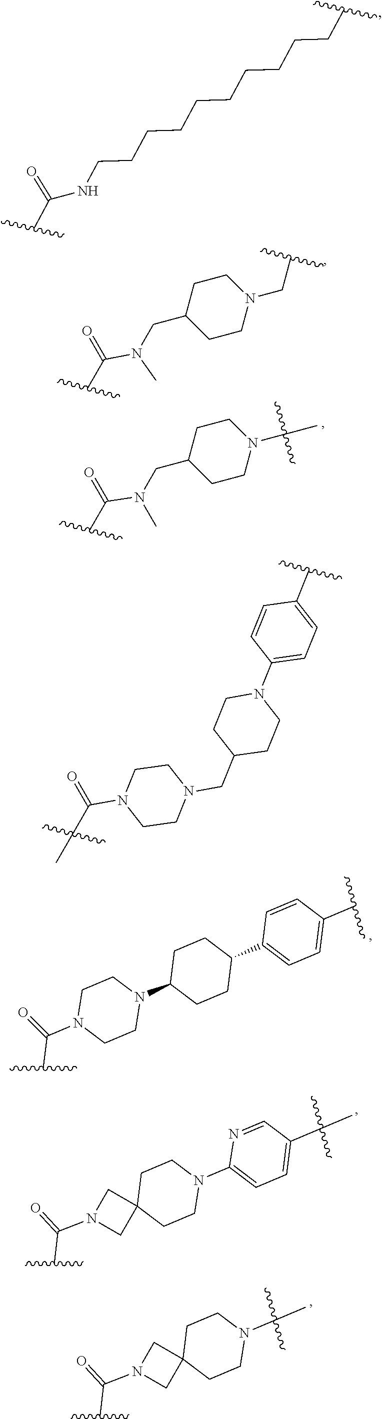

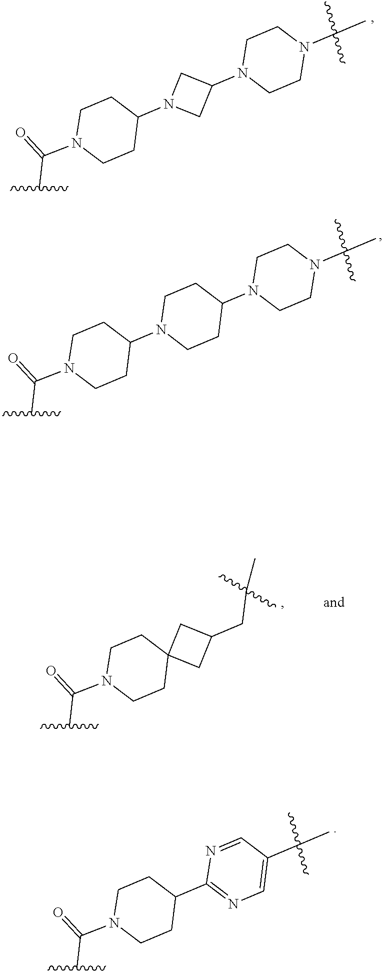 [SMILES: *C(=O)N1CCC(N2CC(N3CCN(*)CC3)C2)CC1 ; *C(=O)N1CCC(N2CCC(N3CCN(*)CC3)CC2)CC1 ; *CC1CC2(CCN(C(*)=O)CC2)C1 ; *C(=O)N1CCC(c2ncc(*)cn2)CC1]

127. The compound of any one of claims 1-110, wherein L comprises a structure selected from the group consisting of:

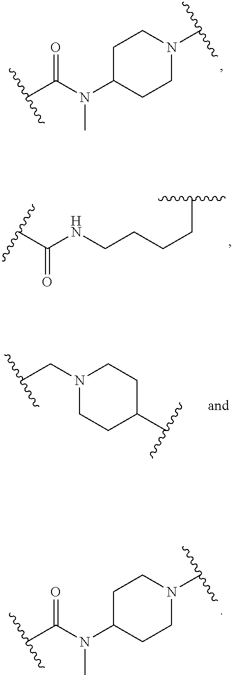 [SMILES: *C(=O)N(C)C1CCN(*)CC1 ; *CCCCNC(*)=O ; *CN1CCC(*)CC1 ; *C(=O)N(C)C1CCN(*)CC1]

128. The compound of any one of claims 1-110, wherein L comprises a structure selected from the group consisting of:

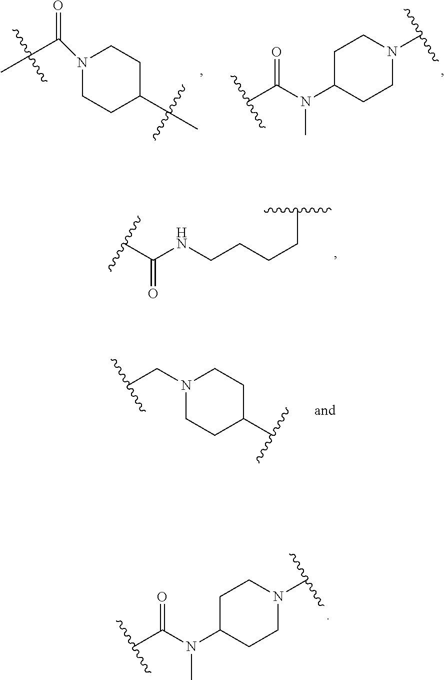 [SMILES: *C(=O)N1CCC(*)CC1 ; *C(=O)N(C)C1CCN(*)CC1 ; *CCCCNC(*)=O ; *CN1CCC(*)CC1 ; *C(=O)N(C)C1CCN(*)CC1]

129. The compound of any one of claims 1-128, wherein 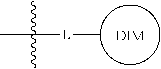
is selected from the group consisting of 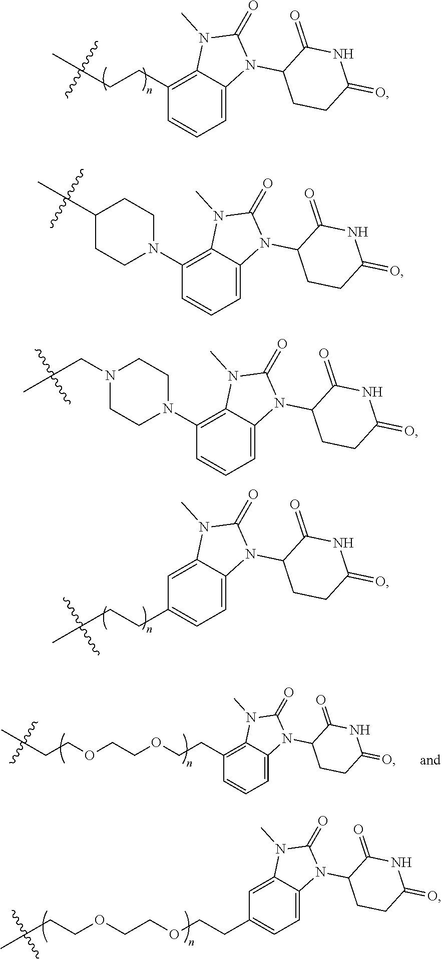
wherein n is an integer from 1 to 8.

130. A compound is represented by Formula (II-ABC′): 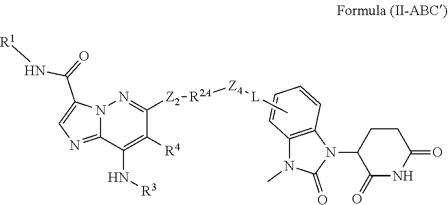
or a pharmaceutically acceptable salt thereof,
wherein:
R¹ is H, —C(CH₃)₂—CN, 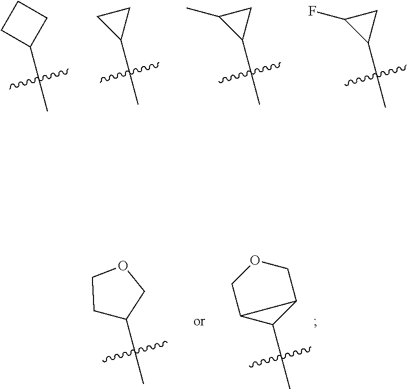
R³ is H or C₁-C₆ aliphatic;
R⁴ is H,
or R³ and R⁴ are joined together to form a 5 or 6-membered heterocyclic ring;
Z₂ is a covalent bond or —NH—;
Z₄ is a covalent bond, —NH—, or —O—;
R^{2A} is 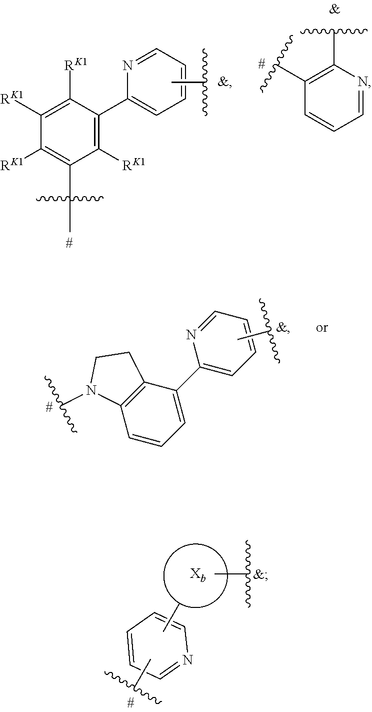
each R^{K1} is independently hydrogen, halo, C₁-C₆ aliphatic, halo substituted C₁-C₆ aliphatic, or —O—(C₁-C₆ aliphatic);
Ring X_{b} is a ring selected from the group consisting of phenyl, a 4 to 7-membered saturated or partially unsaturated carbocyclyl or heterocyclyl with 1-3 heteroatoms independently selected from N, O, and S, and a 5 to 6-membered heteroaryl with 1-4 heteroatoms independently selected from N, O, and S, and each of which is optionally substituted with one or more of R^{K};
each R^{K} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic) 3+, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, a C₁-C₆ aliphatic group, or two R^{K} groups attached to the same carbon or sulfur atom are optionally taken together to form ═O;
n is 1-4;
wherein each #indicates the point of attachment to Z₂ and each & indicates the point of attachment to Z₄; and
L is a covalent bond or a bivalent, saturated or partially unsaturated, straight or branched C₁-C₈ alkylene chain, wherein 0-6 methylene units of L are independently replaced by -Cy-, —O—, —NR^{C}—, —S—, —OC(O)—, —C(O)O—, —C(O)—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—, C₂-C₃ alkynylene and combinations thereof,
wherein -Cy- is independently at each occurrence an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl, having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur, and
each R^{C} is independently at each occurrence hydrogen, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, and combinations thereof.

131. A compound is represented by Formula (II-ABC): 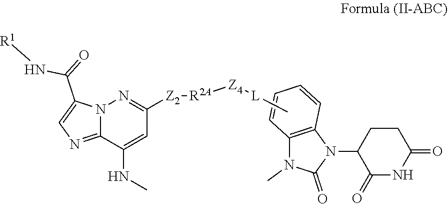
or a pharmaceutically acceptable salt thereof,
wherein:
R¹ is H, —C(CH₃)₂—CN, 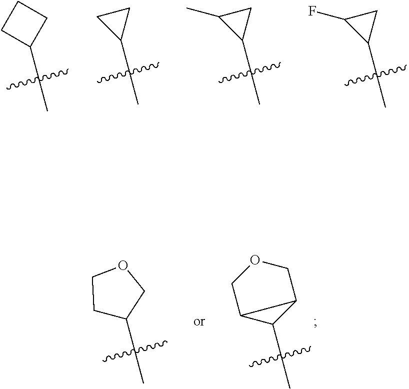
Z₂ is a covalent bond or —NH—;
Z₄ is a covalent bond, —NH—, or —O—;
R^{2A} is 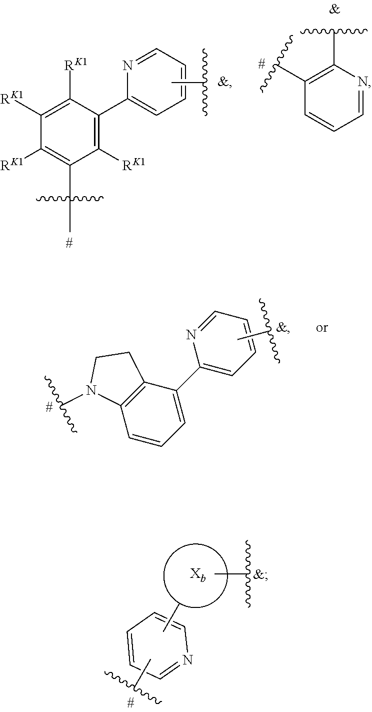
each R^{K1} is independently hydrogen, halo, C₁-C₆ aliphatic, halo substituted C₁-C₆ aliphatic, or —O—(C₁-C₆ aliphatic);
Ring X_{b} is a ring selected from the group consisting of phenyl, a 4 to 7-membered saturated or partially unsaturated carbocyclyl or heterocyclyl with 1-3 heteroatoms independently selected from N, O, and S, and a 5 to 6-membered heteroaryl with 1-4 heteroatoms independently selected from N, O, and S, and each of which is optionally substituted with one or more of R^{K};
each R^{K} is independently hydrogen, —F, —Cl, —Br, —I, —OH, —O—(C₁-C₆ aliphatic), —NO, —NO₂, —NO₃, —O—NO, —N₃, —NH₂, —NH—(C₁-C₆ aliphatic), —N(C₁-C₆ aliphatic)₂, —N(C₁-C₆ aliphatic)₃+, —N(C₁-C₆ aliphatic)-OH, —O—N(C₁-C₆ aliphatic)₂, —N(C₁-C₆ aliphatic)-O—(C₁-C₆ aliphatic), —CN, —NC, —C(O)—C₁-C₆ aliphatic, —CHO, —CO₂H, —CO₂(C₁-C₆ aliphatic), —C(O)—S—(C₁-C₆ aliphatic), —O—C(O)—H, —O—C(O)—(C₁-C₆ aliphatic), —S—C(O)—(C₁-C₆ aliphatic), —C(O)—NH₂, —C(O)—N(C₁-C₆ aliphatic)₂, —C(O)—NHNH₂, —O—C(O)—NHNH₂, —C(S)—NH₂, —C(S)—N(C₁-C₆ aliphatic)₂, —N(C₁-C₆ aliphatic)-CHO, —N(C₁-C₆ aliphatic)-C(O)—(C₁-C₆ aliphatic), —SCN, —NCS, —NSO, —SS(C₁-C₆ aliphatic), —SO₂(C₁-C₆ aliphatic), —SO₂—N(C₁-C₆ aliphatic)₂, —S(O)—O(C₁-C₆ aliphatic), —S(O)—C₁-C₆ aliphatic, —Si(C₁-C₆ aliphatic)₃, —CF₃, —O—CF₃, a C₁-C₆ aliphatic group, or two R^{K} groups attached to the same carbon or sulfur atom are optionally taken together to form ═O;
n is 1-4;
wherein each #indicates the point of attachment to Z₂ and each & indicates the point of attachment to Z₄; and
L is a covalent bond or a bivalent, saturated or partially unsaturated, straight or branched C₁-C₈ alkylene chain, wherein 0-6 methylene units of L are independently replaced by -Cy-, —O—, —NR^{C}—, —S—, —OC(O)—, —C(O)O—, —C(O)—, —S(O)—, —S(O)₂—, —N(R^{C})S(O)₂—, —S(O)₂N(R^{C})—, —N(R^{C})C(O)—, —C(O)N(R^{C})—, —OC(O)N(R^{C})—, and —N(R^{C})C(O)O—, C₂-C₃ alkynylene and combinations thereof,
wherein -Cy- is independently at each occurrence an optionally substituted bivalent ring selected from phenylenyl, an 8-10 membered bicyclic arylenyl, a 4-7 membered saturated or partially unsaturated carbocyclylenyl, a 5-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 5-11 membered saturated or partially unsaturated spiro heterocyclylenyl, having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur, and
each R^{C} is independently at each occurrence hydrogen, or an optionally substituted group selected from a C₁-C₆ aliphatic, phenyl, a 4-7 membered saturated or partially unsaturated heterocyclyl having 1-2 heteroatoms independently selected from N, O, and S, and a 5-6 membered heteroaryl having 1-4 heteroatoms independently selected from N, O, and S, and combinations thereof.

132. The compound of claim 130 or 131, wherein L is bivalent, saturated or partially unsaturated, straight or branched C₁-C₈ alkylene chain, wherein 0-6 methylene units of L are independently replaced by -Cy-, —O—, —NR^{C}—, —C(O)—, C₂-C₃ alkynylene, and combinations thereof and combinations thereof.

133. The compound of any one of claims 130-132, wherein R¹ is

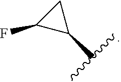 [SMILES: *[C@@H]1C[C@@H]1F]

134. The compound of any one of claims 130-133, wherein the compound is represented by Formula (II-ABC-I):

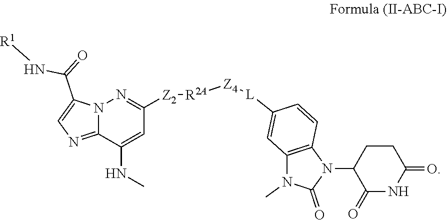

135. The compound of any one of claims 130-134, wherein the compound is represented by Formula (II-ABC-II):

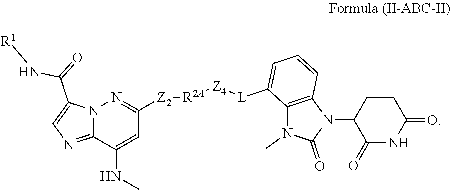

136. The compound of any one of claims 130-135, wherein L comprises a structure selected from the group consisting of: 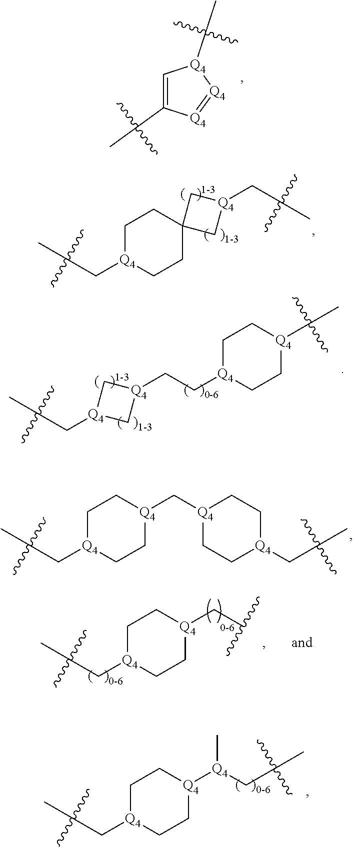
where each Q₄ is independently selected from —CH— and —N— when Q₄ is attached to only single bonds, or Q₄ is ═CH— or ═N— when Q₄ is attached to a double bond.

137. The compound of of any one of claims 130-136, wherein L comprises a structure selected from the group consisting of:

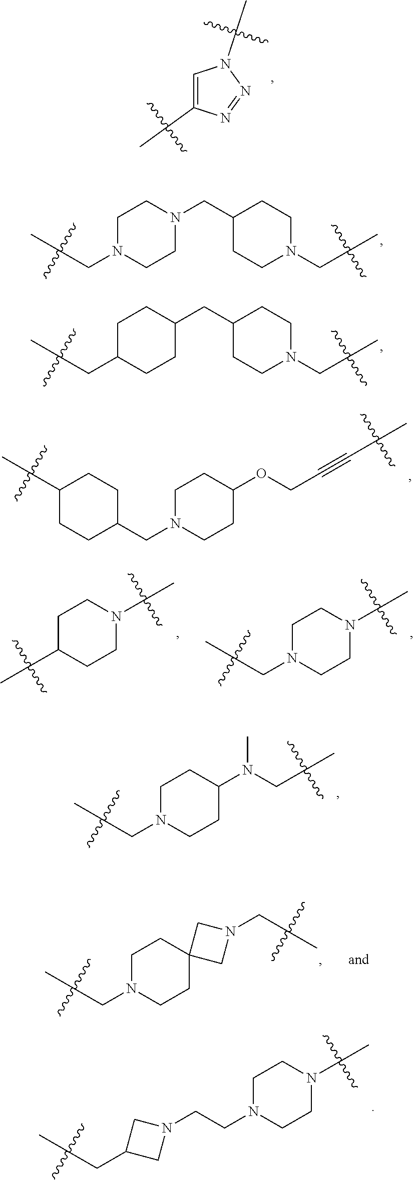

138. The compound of any one of claims 130-136, wherein L comprises a structure selected from the group consisting of:

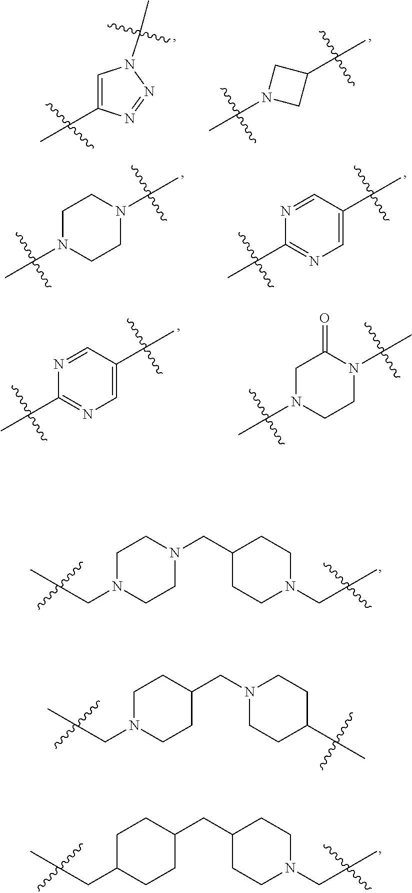

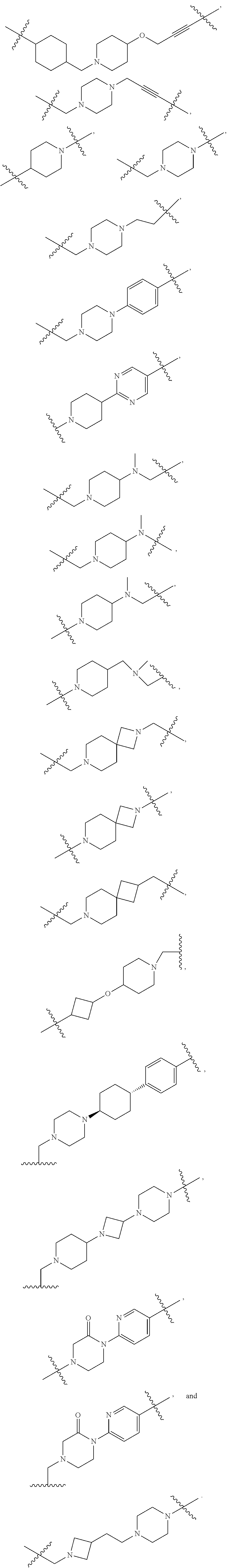

139. The compound of of any one of claims 130-136, wherein L comprises a structure selected from the group consisting of:

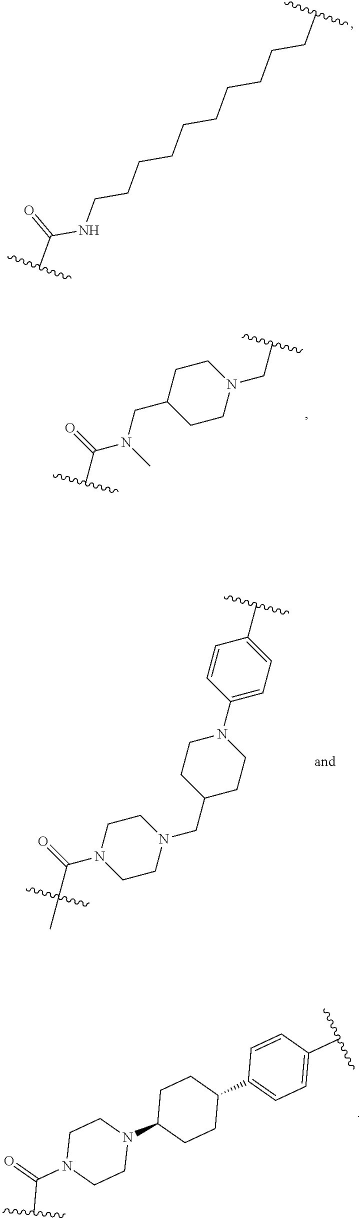 [SMILES: *CCCCCCCCCCNC(*)=O ; *CN1CCC(CN(C)C(*)=O)CC1 ; *C(=O)N1CCN(CC2CCN(c3ccc(*)cc3)CC2)CC1 ; *C(=O)N1CCN([C@H]2CC[C@H](c3ccc(*)cc3)CC2)CC1]

140. The compound of of any one of claims 130-136, wherein L comprises a structure selected from the group consisting of:

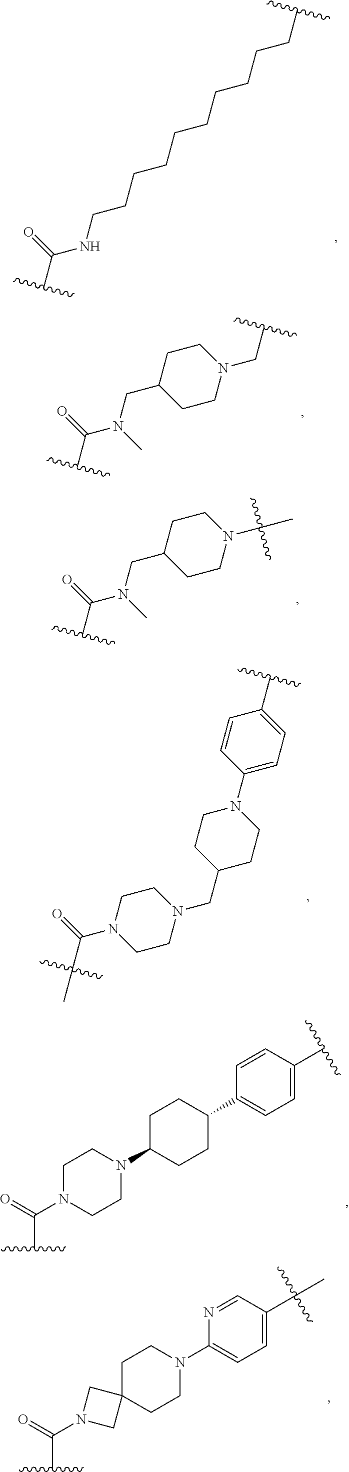 [SMILES: *CCCCCCCCCCNC(*)=O ; *CN1CCC(CN(C)C(*)=O)CC1 ; *C(=O)N(C)CC1CCN(*)CC1 ; *C(=O)N1CCN(CC2CCN(c3ccc(*)cc3)CC2)CC1 ; *C(=O)N1CCN([C@H]2CC[C@H](c3ccc(*)cc3)CC2)CC1 ; *C(=O)N1CC2(CCN(c3ccc(*)cn3)CC2)C1]

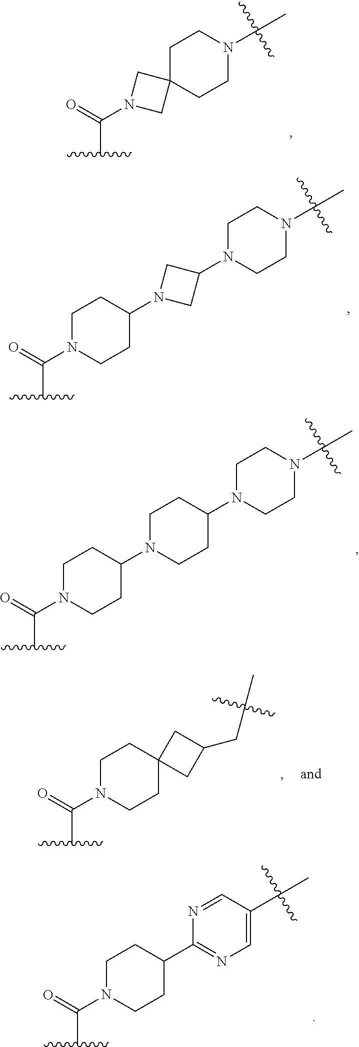 [SMILES: *C(=O)N1CC2(CCN(*)CC2)C1 ; *C(=O)N1CCC(N2CC(N3CCN(*)CC3)C2)CC1 ; *C(=O)N1CCC(N2CCC(N3CCN(*)CC3)CC2)CC1 ; *CC1CC2(CCN(C(*)=O)CC2)C1 ; *C(=O)N1CCC(c2ncc(*)cn2)CC1]

141. The compound of any one of claims 130-136, wherein L comprises a structure selected from the group consisting of:

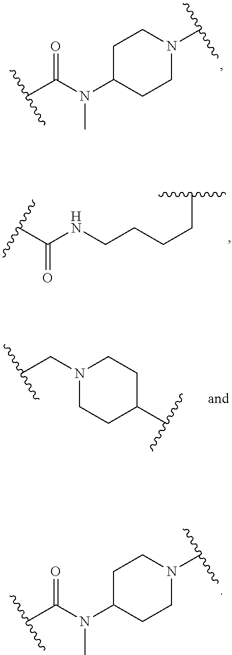 [SMILES: *C(=O)N(C)C1CCN(*)CC1 ; *CCCCNC(*)=O ; *CN1CCC(*)CC1 ; *C(=O)N(C)C1CCN(*)CC1]

142. The compound of any one of claims 130-136, wherein L comprises a structure selected from the group consisting of:

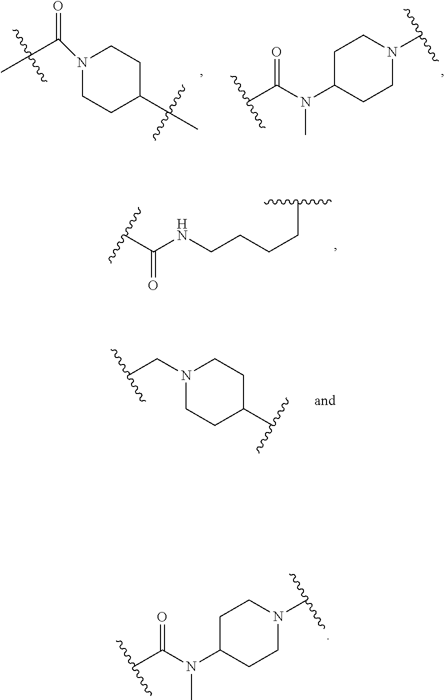 [SMILES: *C(=O)N1CCC(*)CC1 ; *C(=O)N(C)C1CCN(*)CC1 ; *CCCCNC(*)=O ; *CN1CCC(*)CC1 ; *C(=O)N(C)C1CCN(*)CC1]

143. The compound of any one of claims 1-142, wherein the compound is selected from any one of the compounds depicted in Table 1, or a pharmaceutically acceptable salt thereof.

144. A pharmaceutical composition comprising the compound according to claim 143, and a pharmaceutically acceptable carrier, adjuvant, or vehicle.

145. The pharmaceutical composition according to claim 144, further comprising an additional therapeutic agent.

146. The pharmaceutical composition of claim 144 or 145, for use in the manufacture of a medicament for the treatment of a TYK2 mediated disorder, disease, or condition in a patient.

147. A pharmaceutical dosage form comprising the compound according to claim any one of claims 1-143 or the pharmaceutical composition of any one of claims 144-146.

148. A method of modulating TYK2 in a subject or biological sample comprising administering to the subject, or contacting the biological sample with the compound according to any one of claims 1-0, or the pharmaceutical composition of any one of claims 144-146, or the pharmaceutical dosage form of claim 147.

149. The method of claim 148, wherein modulating TYK2 comprises inhibiting or degrading TYK2.

150. A method of treating a TYK2-mediated disorder, disease, or condition in a subject in need thereof comprising administering to the subject the compound according to any of one claims 1-143, or the pharmaceutical composition of any one of claims-144-146, or the pharmaceutical dosage form of claim 147.

151. The method of claim 150, wherein the TYK2-mediated disorder is an autoimmune disorder, an inflammatory disorder, a proliferative disorder, an endocrine disorder, a neurological disorder, or a disorder associated with transplantation.

152. The method of claim 150, wherein the TYK2-mediated disorder is an autoimmune disorder selected from type 1 diabetes, ankylosing spondylitis, cutaneous lupus erythematosus, systemic lupus erythematosus, multiple sclerosis, systemic sclerosis, psoriasis, Crohn's disease, ulcerative colitis, and inflammatory bowel disease.

153. The method of claim 150, wherein the TYK2-mediated disorder is an inflammatory disorder selected from rheumatoid arthritis, asthma, chronic obstructive pulmonary disease, psoriasis, Crohn's disease, ulcerative colitis, and inflammatory bowel disease.

154. The method of claim 150, wherein the TYK2-mediated disorder is a proliferative disorder selected from a hematological cancer, polycythemia vera, myelofibrosis, essential thrombocythemia, and thrombocytosis.

155. The method of claim 150, wherein the TYK2-mediated disorder is an endocrine disorder selected from polycystic ovary syndrome, Crouzon's syndrome, and type 1 diabetes.

156. The method of claim 150, wherein the TYK2-mediated disorder is a neurological disorder selected from Alzheimer's disease, Parkinson's disease, amyotrophic lateral sclerosis, Huntington's disease, cerebral ischemia, and neurodegenerative disease caused by traumatic injury, glutamate neurotoxicity and hypoxia.

157. The method of claim 150, wherein the TYK2-mediated disorder is a disorder is associated with transplantation selected from transplant rejection and graft versus host disease.

158. The method of any one of claims 150-157, wherein the subject is human.
